(19) United States
(12) Patent Application Publication
(10) Pub. No.: US 2026/0297060 A1
(43) Pub. Date: Oct. 1, 2026

(54) COMPOUNDS FOR THE DEGRADATION OF MUTANT BRAF

(71) Applicant: C4 THERAPEUTICS, INC., Watertown, MA (US)

(72) Inventors: Katrina L. Jackson, Weston, MA (US); Yanke Liang, Belmont, MA (US); Ronan Patrick Hanley, Waltham, MA (US); Bridget Kreger, Acton, MA (US); Roy MacFarlane Pollock, Medford, MA (US)

(73) Assignee: C4 THERAPEUTICS, INC., Watertown, MA (US)

(21) Appl. No.: 19/664,691

(22) Filed: Apr. 30, 2026

Related U.S. Application Data
(63) Continuation of application No. PCT/US2024/054007, filed on Oct. 31, 2024.
(60) Provisional application No. 63/546,727, filed on Oct. 31, 2023.
(60) Provisional application No. 63/599,455, filed on Nov. 15, 2023.

(51) Int. Cl. C07D 401/14; C07D 403/14; C07D 407/14; C07D 491/08; C07D 491/107

(52) U.S. Cl. CPC C07D 401/14; C07D 403/14; C07D 407/14; C07D 491/08; C07D 491/107

(57) ABSTRACT

The present invention provides compounds or their pharmaceutically acceptable salts and their pharmaceutical compositions that can be administered to a host such as a human in need thereof for the treatment of a disorder, such as cancer, mediated by mutant BRAF. The compounds efficiently degrade Class I, II and III mutant BRAF proteins.

### CROSS REFERENCE TO RELATED APPLICATIONS

This application is a continuation of International Patent Application No. PCT/US2024/054007, filed Oct. 31, 2024, which claims the benefit of U.S. provisional application 63/546,727, filed Oct. 31, 2023, and 63/599,455 filed Nov. 15, 2023. The entirety of each of these applications is hereby incorporated by reference for all purposes.

### FIELD OF THE INVENTION

The present invention provides compounds and their pharmaceutically acceptable salts, uses, compositions and manufacture that degrade mutant BRAF, such as Class I, Class II, and/or Class III mutant BRAF. The compounds of the present invention can be administered to a host such as a human in need thereof for the therapeutic treatment of a disorder, such as cancer, mediated by mutant BRAF.

### BACKGROUND

BRAF is a serine/threonine protein kinase that is a member of the signal transduction protein kinases. BRAF plays a critical role in the MAPK signaling pathway and is mutated in approximately 8% of all human cancers including melanoma (460%), colorectal carcinoma (~12%), and lung adenocarcinoma (~5%). The most common mutation in BRAF is V600E (Class I), which occurs in half of malignant melanomas. This mutation hyperactivates ERK and signals as a RAF inhibitor-sensitive monomer. Other common activating mutations include Class II mutations such as G469A and Class III mutations such as G466V. Class II and III mutations activate ERK by promoting RAF homo- or hetero-dimerization.

Despite the therapeutic benefits of available BRAF inhibitors, the duration of the antitumor response to these drugs can be limited by the acquisition of drug resistance.

The BRAF protein presents a mechanism for signaling propagation that requires protein homo-dimerization (BRAF-BRAF) or hetero-dimerization with other RAF proteins (BRAF-RAF1 or BRAF-ARAF). When BRAF has a Class I mutation, as observed in oncology indications with BRAF V600E/K substitution, BRAF signaling becomes independent of homodimers and/or heterodimers. The kinase activity becomes hyperactivated as a monomeric protein and drives cellular proliferative signals.

Several BRAF inhibitors have been described that can inhibit monomeric BRAF but not dimeric BRAF including vemurafenib, dabrafenib, and encorafenib, however, resistance usually emerges within a year, including RAS mutation, BRAF V600E amplification, and BRAF V600E intragenic deletion or splice variants. These inhibitors are also ineffective against non-V600 BRAF mutants (Class II & III) that activate ERK by promoting RAF homo-or hetero-dimerization.

Examples of BRAF inhibitors are described in WO2021/116055, WO2021/116050, WO2022/129259, and WO2022/258584.

Non-limiting examples of BRAF degrading compounds include those described in WO2018/119448, WO2019/199816, WO2020/051564, WO2021/255212, WO2022/047145, WO2022/261250, and WO2024/119111.

Despite these efforts there remains a need for new therapeutic drugs to treat BRAF mediated cancers, and in particular drugs that treat mutant BRAF mediated cancers.

### SUMMARY

The present invention provides compounds and their pharmaceutically acceptable salts, uses, compositions, and manufacture that degrade mutant BRAF, for example a Class I, Class II, and/or Class III mutant BRAF, via the ubiquitin proteasome pathway. These compounds bind to the ubiquitously expressed E3 ligase protein cereblon (CRBN) and alter the substrate specificity of the CRBN E3 ubiquitin ligase complex, resulting in the recruitment and ubiquitination of mutant BRAF, such as for example BRAF V600E. The present compounds are also biochemical binders of other RAF proteins, for example WT BRAF, RAF1 or ARAF, however more effective targeted degradation is triggered by these compounds for mutant BRAF, such as for example Class I mutant BRAF such as V600E, Class II mutant BRAF such as G469A, Class III mutant BRAF such as G466V mutations, and splice variants such as p61-BRAF^{ZV600E}.

By degrading mutant BRAF a compound of the present invention can be used to treat a mutant BRAF mediated cancer, for example melanoma, lung cancer including for example non-small cell lung cancer, colorectal cancer including for example microsatellite stable colorectal cancer, thyroid cancer including for example anaplastic thyroid cancer, or ovarian cancer. In certain embodiments a compound of the present invention is used to treat a solid tumor that is mediated by a V600X mutant BRAF. Additional non-limiting examples of disorders that can be treated with the compounds of the present invention include melanoma, non-small cell lung carcinoma, thyroid cancer, colorectal cancer, and other solid tumor malignancies that have a mutant BRAF driver.

In certain aspects selected compounds of the present invention provide a fast and durable degradation of mutant BRAF protein. By degrading mutant BRAF these compounds may be used in the treatment of mutant BRAF mediated cancer. For example, in A375 human melanoma xenografts, oral delivery of Compound 8, Compound 118, Compound 141, Compound 156, or Compound 180 results in a more robust antitumor response than comparators such as dabrafenib or encorafenib (see e.g., FIGS. 14 and 22). These compounds cause profound tumor regressions in the xenograft model when dosed at 10 mg/kg BID.

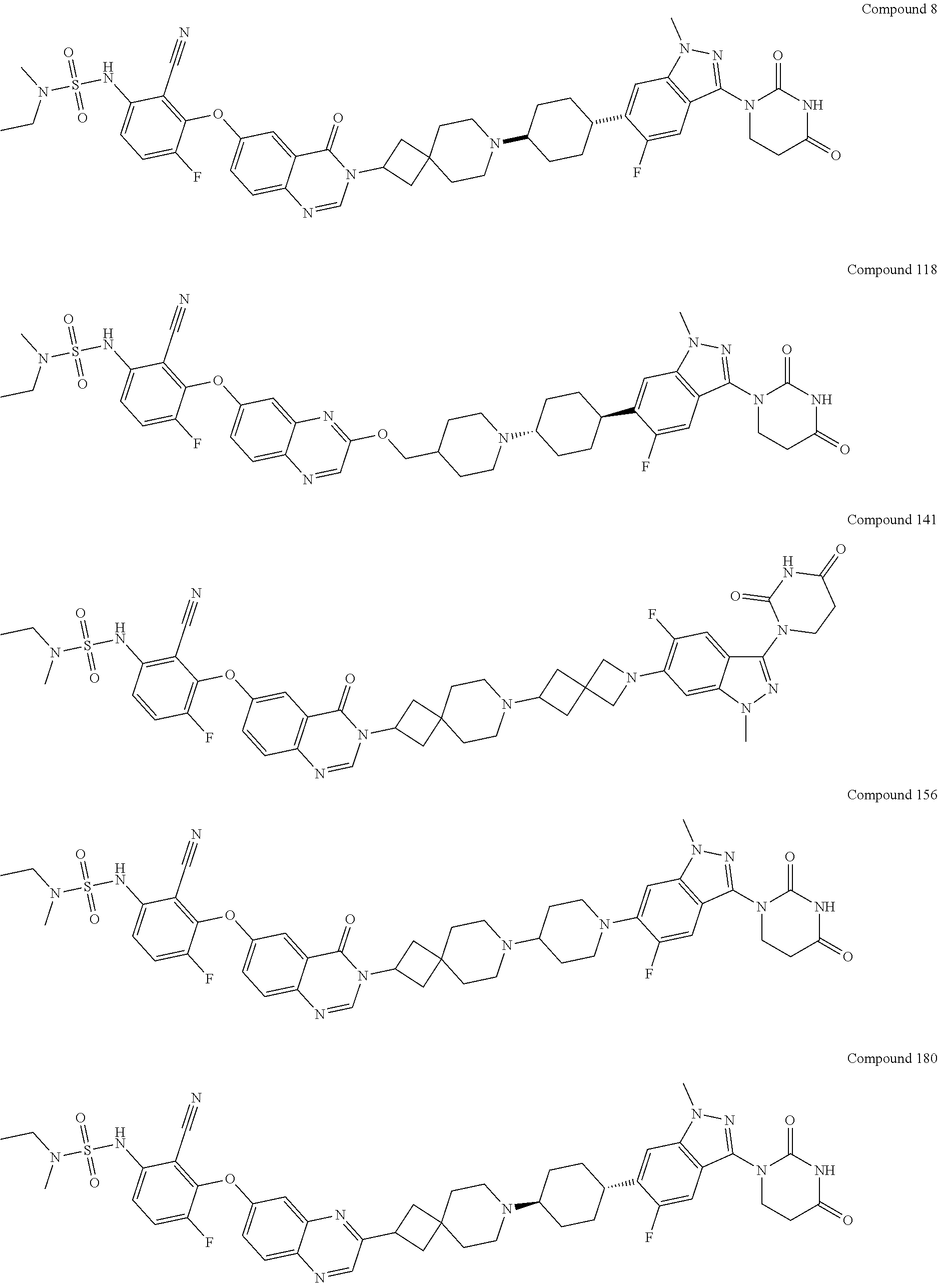

A compound of the present invention can be used to treat difficult to treat double mutant cancers wherein one oncogenic mutation is in BRAF. For example, in certain embodiments a compound of the present invention is used to treat a cancer which has a BRAF mutation and a PI3K, NRAS, or MEK1 mutation. In certain embodiments the PI3K mutation is a subunit mutation, for example a mutation to the p110 alpha protein such as PIK3CA^{H1047R} or PIK3CAP^{449T}.

A compound of the present invention can be used to degrade BRAF mutants of Class I, Class II, Class III, and splice variants thereof. In certain embodiments a compound of the present invention can treat a cancer that has developed resistance to a BRAF inhibitor. For example, in certain embodiments a compound of the present invention is used to treat a cancer that has acquired resistance to BRAF inhibitor treatment by gaining a NRAS, MEK1, or PI3K mutation in addition to having a BRAF mutation.

In certain embodiments a compound of the present invention is orally bioavailable.

In certain embodiments, a compound of Formula I to Formula XXXII is provided:

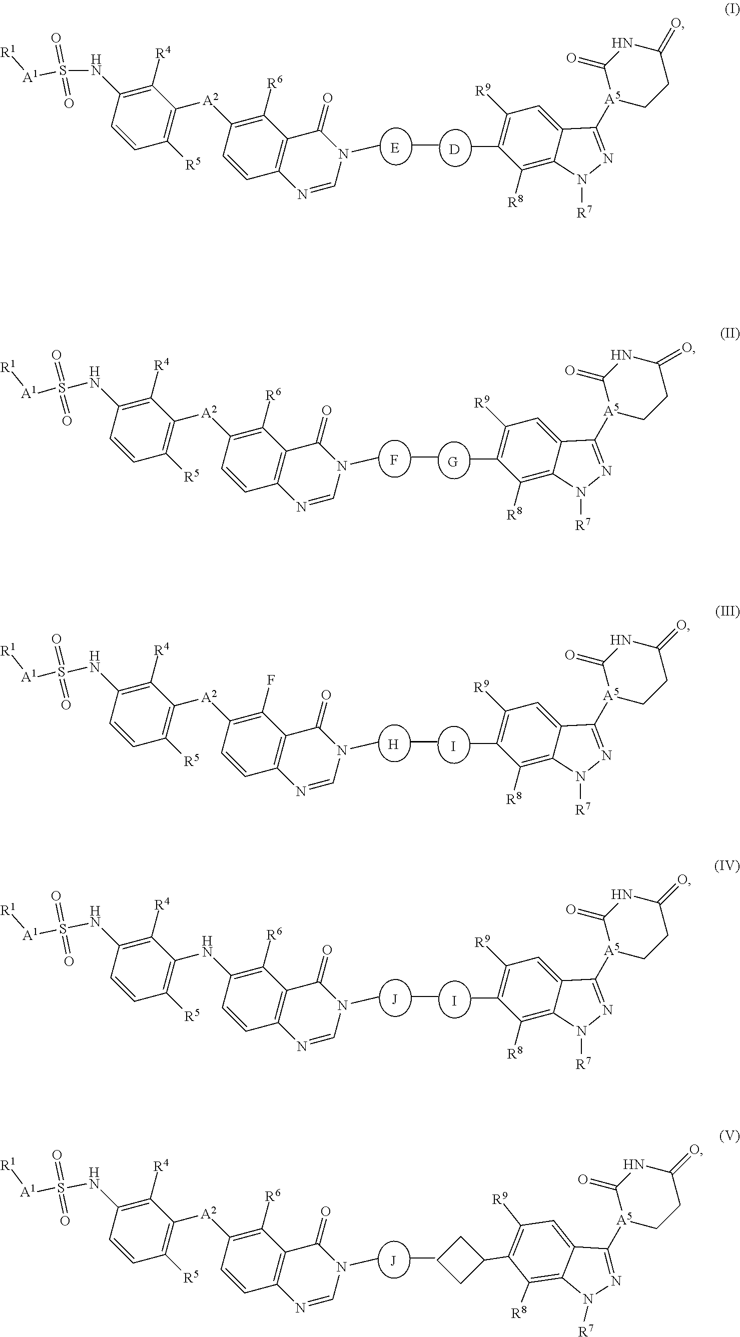

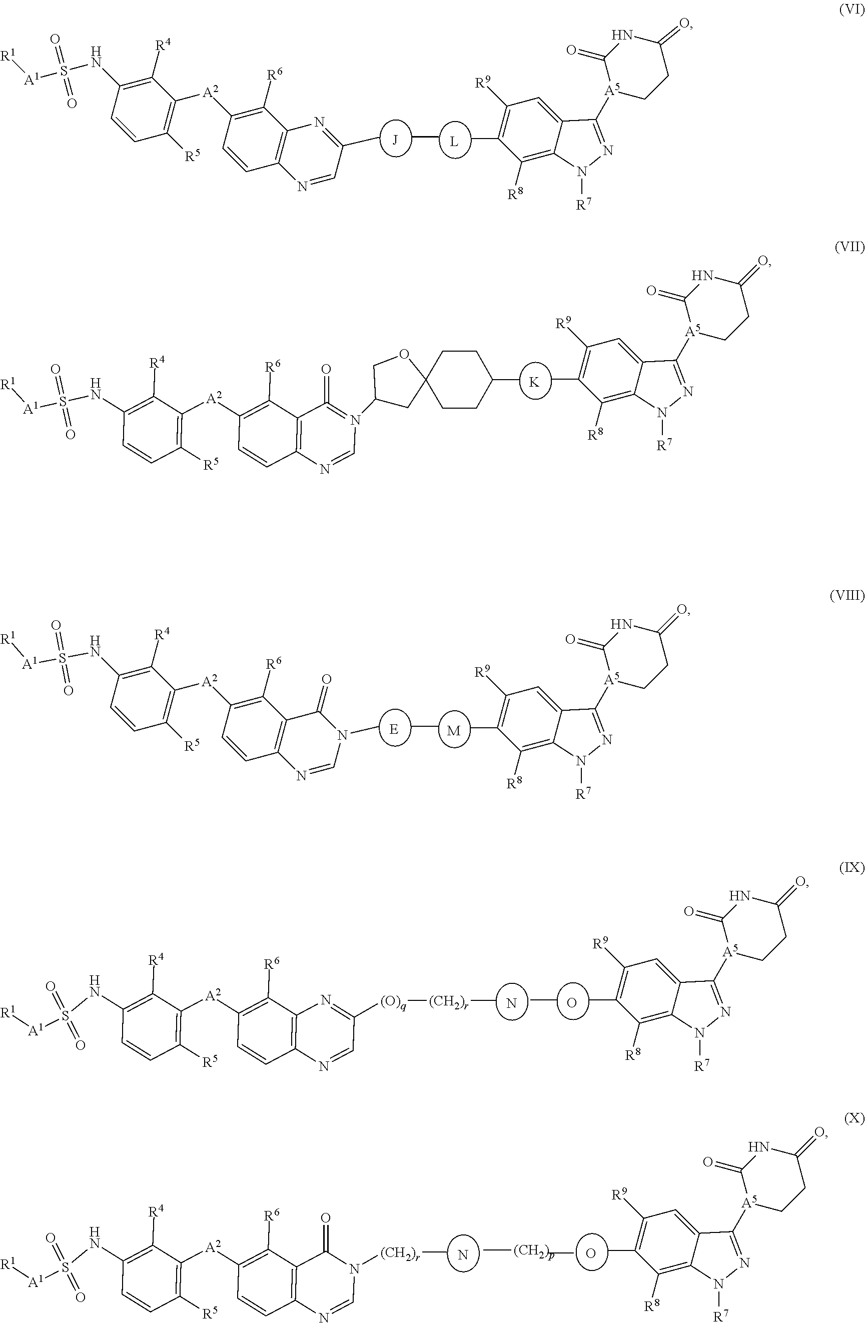

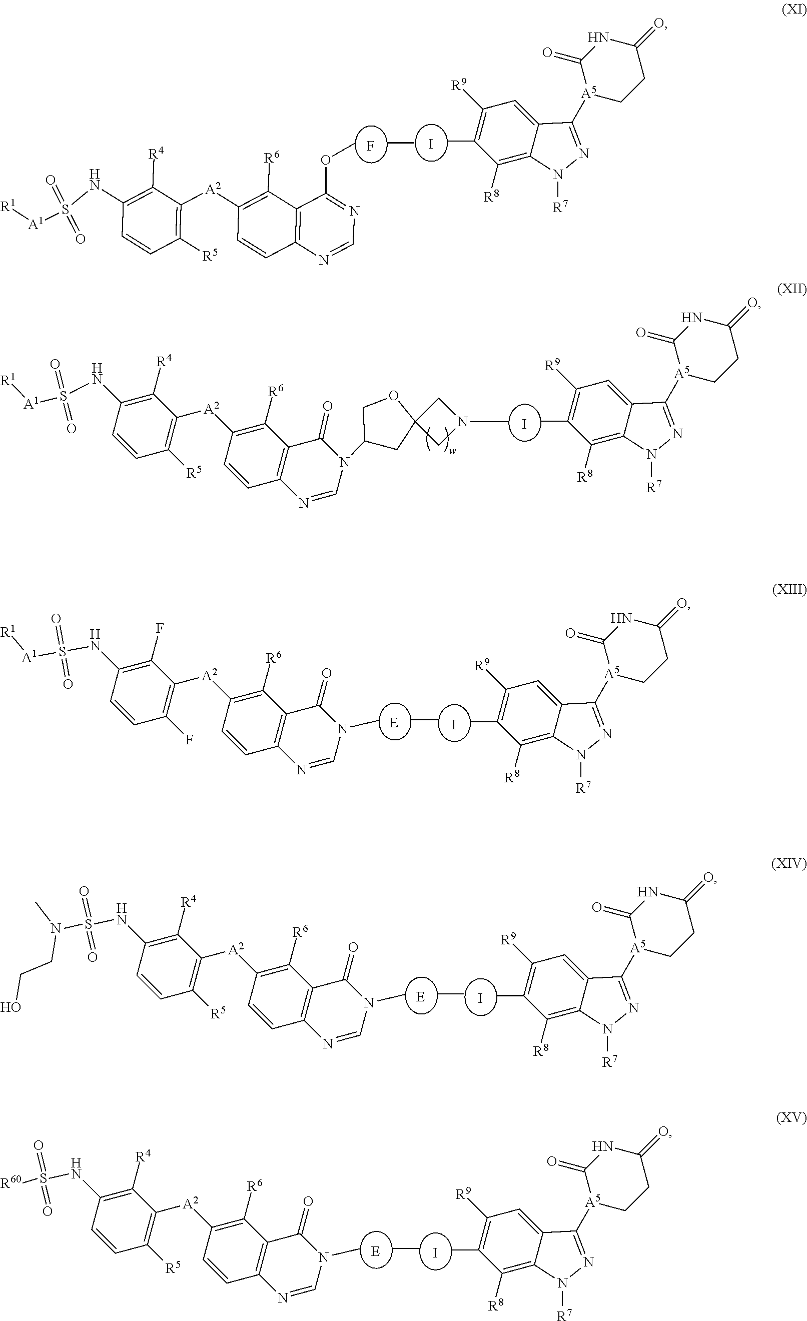

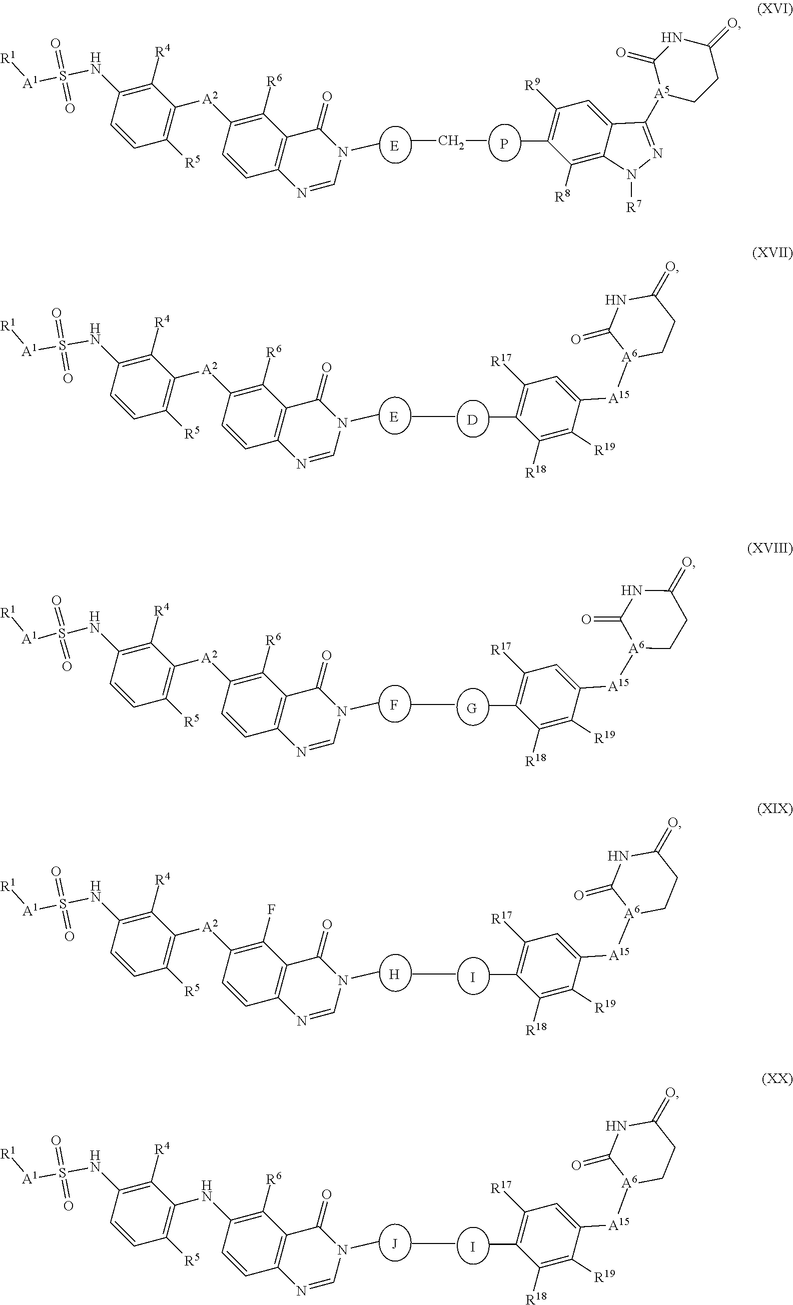

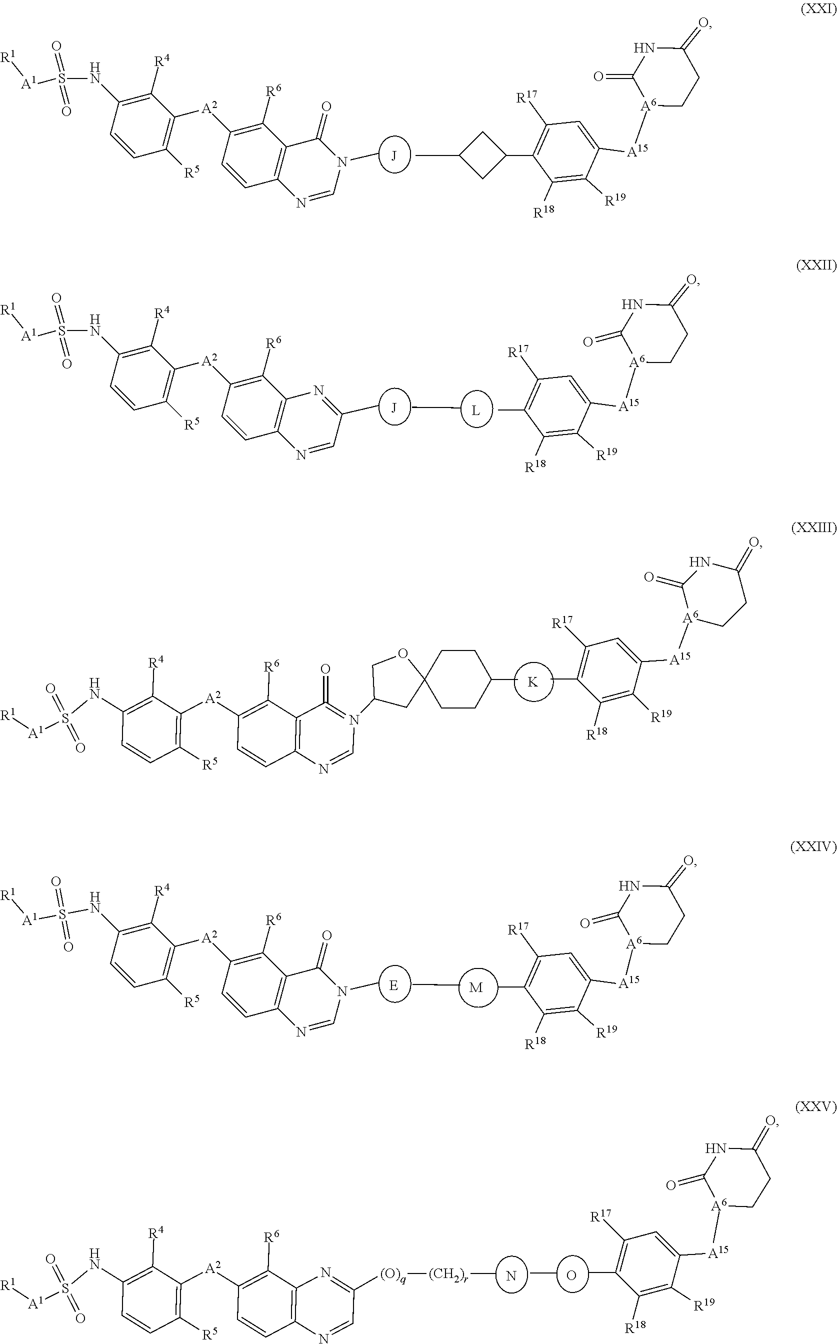

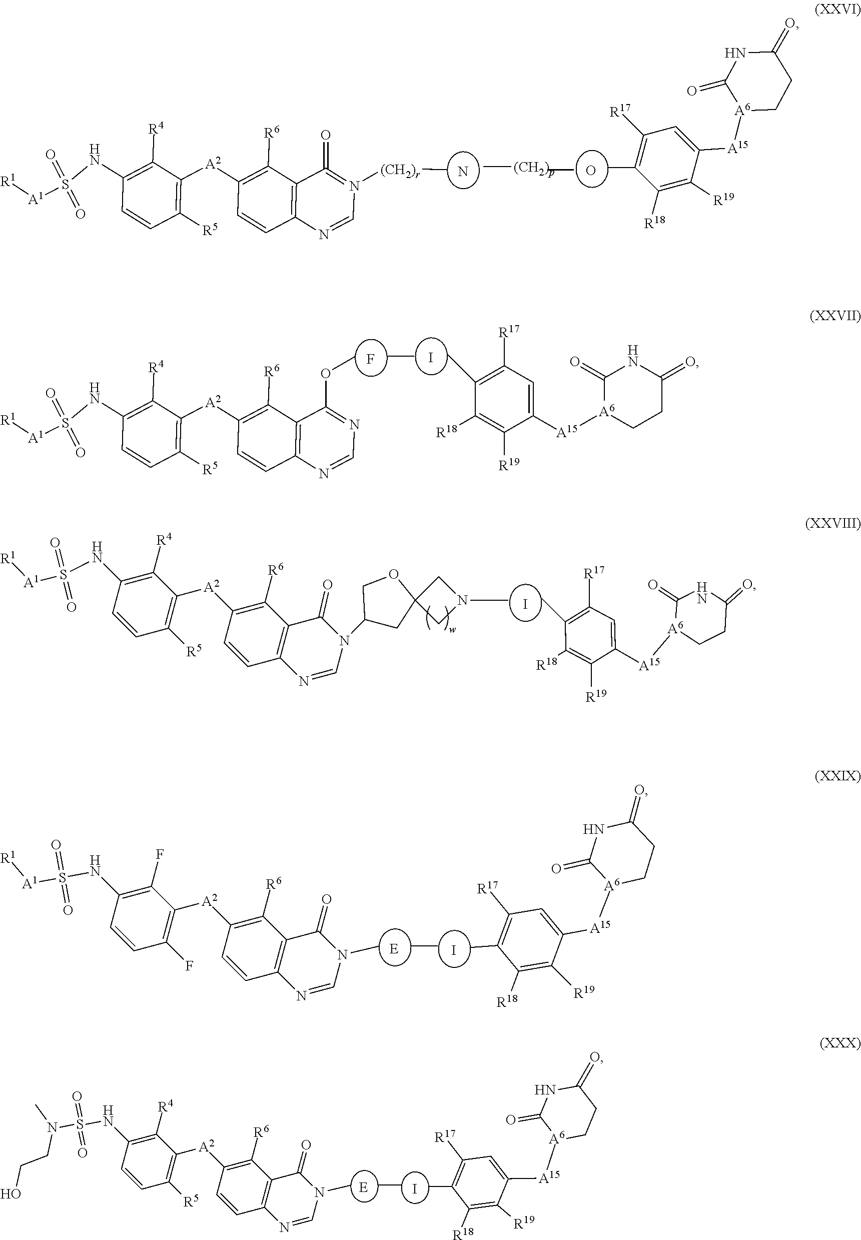

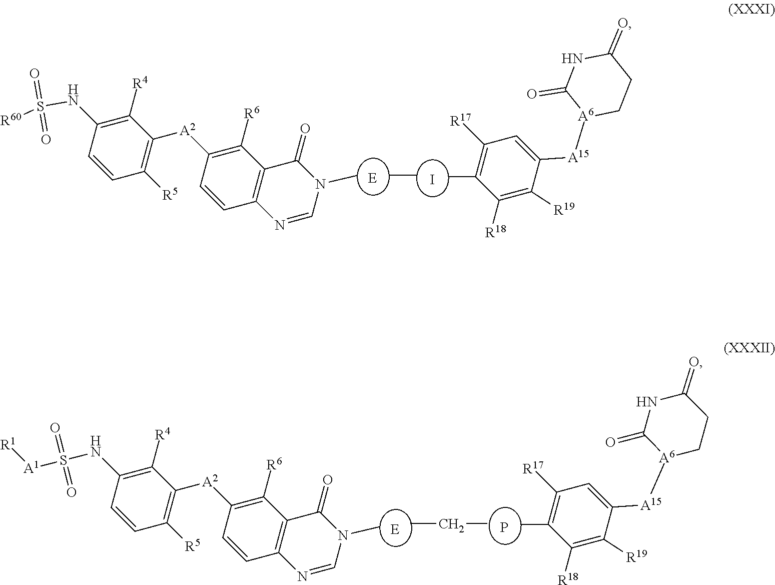

or pharmaceutically acceptble salt thereof,
   wherein:
   Cycle \textcircled{D} is a spiroheterocyclyl containing 1 or 2 nitrogen atoms and containing no other heteroatoms, bridged cycloalkyl, bridged heterocyclyl, or piperidine-1,4-diyl, wherein cycle \textcircled{D} is optionally substituted with one or two substituents independently selected from halogen, alkyl and alkoxy;
   Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
   Cycle \textcircled{F} is a bridged heterocyclyl, fused heterocyclyl, or spiroheterocyclyl containing 1 or 2 nitrogen atoms and containing no other heteroatoms, wherein cycle \textcircled{F} is optionally substituted with one or two substituents independently selected from halogen, alkyl and alkoxy;
   Cycle \textcircled{G} is a cycloalkyl or heterocyclyl containing 1 or 2 nitrogen atoms and 0 or 1 oxygen atom and containing no other heteroatoms, wherein cycle \textcircled{G} is optionally substituted with one or two substituents independently selected from halogen, alkyl and alkoxy;
   Cycle \textcircled{H} is a spiroheterocyclyl containing 1 oxygen atom and 0 or 1 nitrogen atom and containing no other heteroatoms, wherein cycle \textcircled{H} is optionally substituted with one or two substituents independently selected from halogen, alkyl and alkoxy;
   Cycle \textcircled{I} is a cycloalkyl, wherein cycle \textcircled{I} is optionally substituted with one or two substituents independently selected from halogen, alkyl and alkoxy;
   Cycle \textcircled{J} is a spiroheterocyclyl containing 1 or 2 nitrogen atoms and 0 or 1 oxygen atoms and containing no other heteroatoms, wherein cycle \textcircled{J} is optionally substituted with one or two substituents independently selected from halogen, alkyl and alkoxy;
   Cycle \textcircled{k} is a cycloalkyl, 3,3-difluoro-piperidine-1,4-diyl, piperazine-1,4-diyl, bridged cycloalkyl, bridged heterocyclyl, or a spiroheterocyclyl containing 1 or 2 nitrogen atoms and 0 or 1 oxygen atom and containing no other heteroatoms, wherein the cycloalkyl piperazine-1,4-diyl, or a spiroheterocyclyl is optionally substituted with one or two substituents independently selected from halogen, alkyl and alkoxy;
   Cycle \textcircled{L} is a cycloalkyl, piperidine-1,4-diyl, spiroheterocyclyl containing 1 or 2 nitrogen atoms and containing no other heteroatoms, wherein cycle \textcircled{L} is optionally substituted with one or two substituents independently selected from halogen, alkyl and alkoxy;
   Cycle \textcircled{M} is a cycloalkyl substituted with 1, 2, or 3 substituents independently selected from the group consisting of alkyl, alkoxy, F, Cl, Br, and OH;
   Cycle \textcircled{N} is a cycloalkyl, spirocycloalkyl, heterocyclyl containing 1 or 2 nitrogen atoms and containing no other heteroatoms, wherein cycle \textcircled{N} is optionally substituted with one or two substituents independently selected from halogen, alkyl and alkoxy;
   Cycle \textcircled{O} is a cycloalkyl, spirocycloalkyl, heterocyclyl containing 1 or 2 nitrogen atoms and containing no other heteroatoms, wherein cycle \textcircled{O} is optionally substituted with one or two substituents independently selected from halogen, alkyl and alkoxy;
   Cycle \textcircled{P} is a heterocyclyl optionally substituted with one or two substituents independently selected from halogen, alkyl OH and alkoxy;
   A¹ is selected from —NR²— and —CHR^{2′}—;
   R¹ is selected from hydrogen, alkyl and cycloalkyl;
   R² is selected from hydrogen, alkyl, cycloalkyl and haloalkyl;
   or R¹ and R² together with the nitrogen atom to which they are attached form heterocycloalkyl optionally substituted with one or two R³;
   R^{2′} is selected from hydrogen, alkyl, cycloalkyl and haloalkyl;
   or R¹ and R^{2′} together with the carbon atom to which they are attached form cycloalkyl optionally substituted with one or two R³;
   each R³ is independently selected from hydrogen, halogen, alkyl, cycloalkyl and alkoxy;
   R⁴ is selected from hydrogen, alkyl, cyano and halogen;
   R⁵ is selected from hydrogen, alkyl, cyano and halogen;
   A² is selected from —O—, —NH— and —(C═O)—;
   R⁶ is selected from hydrogen, halogen, hydroxy, amino, dialkylamino, alkoxy, alkyl and alkoxyalkyl;
   R⁷ is selected from hydrogen and alkyl;
   R⁸ is selected from hydrogen, alkyl, cyano, halogen, and alkoxy;
   R⁹ is selected from hydrogen, alkyl, cyano, halogen, and alkoxy;
   R¹⁷ is selected from hydrogen, alkyl, cyano, hydroxy, cycloalkyl, halogen and alkoxy;
   R¹⁸ is selected from hydrogen, alkyl, cyano, hydroxy, cycloalkyl, halogen and alkoxy;
   R¹⁹ is selected from hydrogen, alkyl, cyano, hydroxy, cycloalkyl, halogen and alkoxy;
   A⁵ is —CH— or —N—;
   A¹⁵ is selected from a bond, —O— and —NH—, wherein A⁶ is CH when A¹⁵ is O or NH and wherein A⁶ is N or CH when A¹⁵ is bond;
   A⁶ is —CH— or —N—;
   R⁶⁰ is isopropyl; pyrrolidine-1-yl substituted with F or OMe; or cyclopropyl;
   q is 0 or 1;
   r is 0, 1 or 2;
   p is 0, 1 or 2;
   w is 1 or 2.

### Non-Limiting Examples of Compounds of the Invention Include

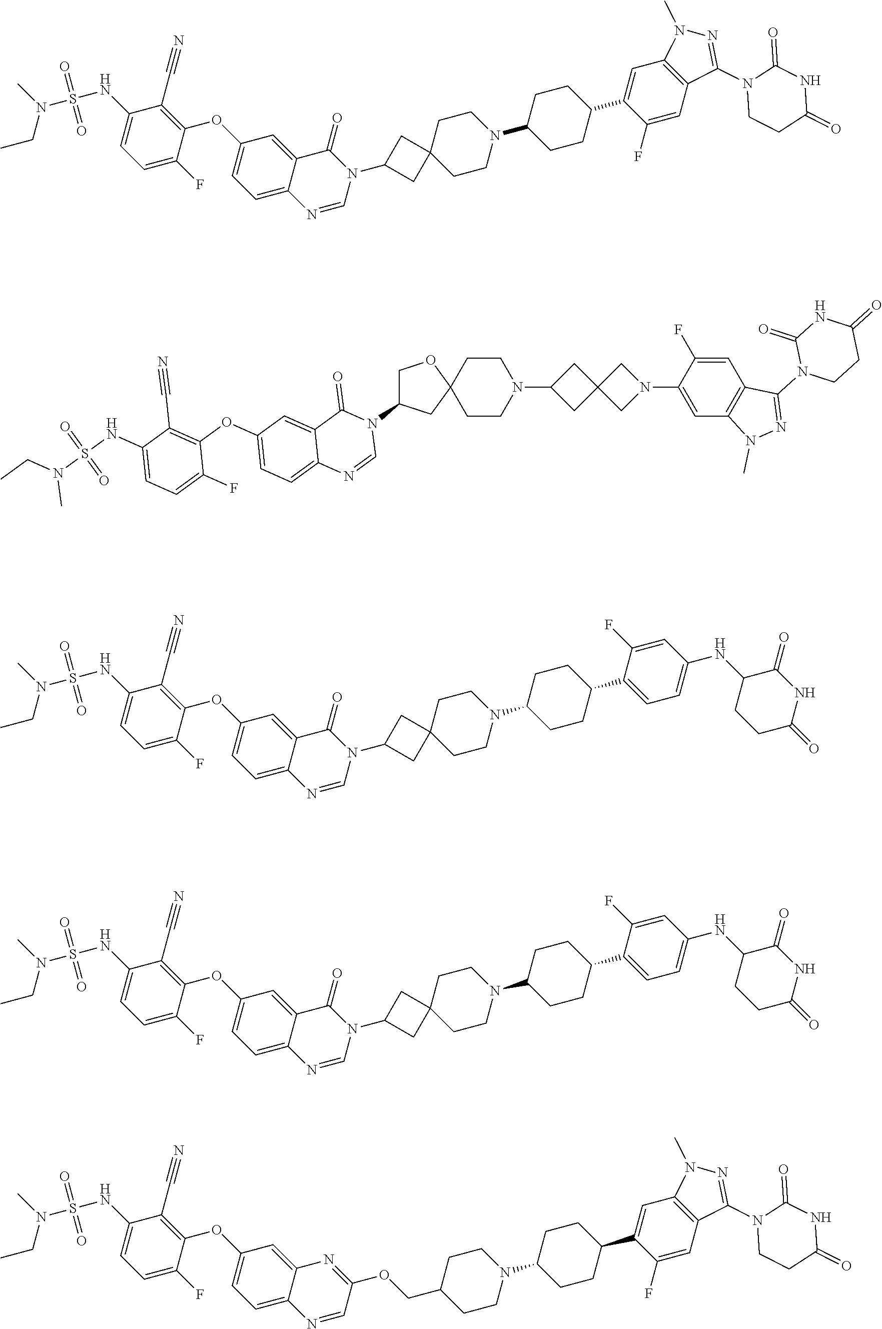

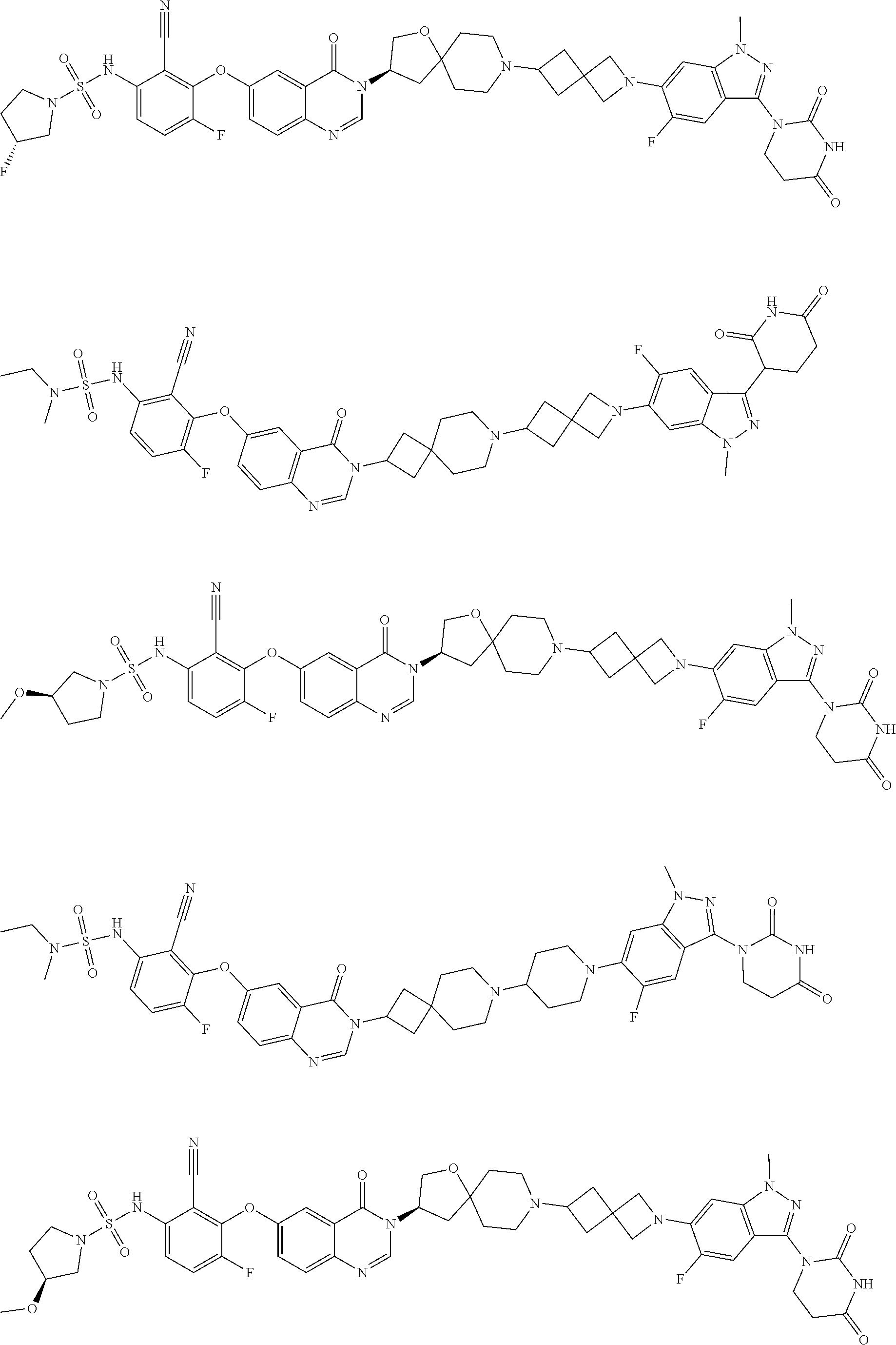

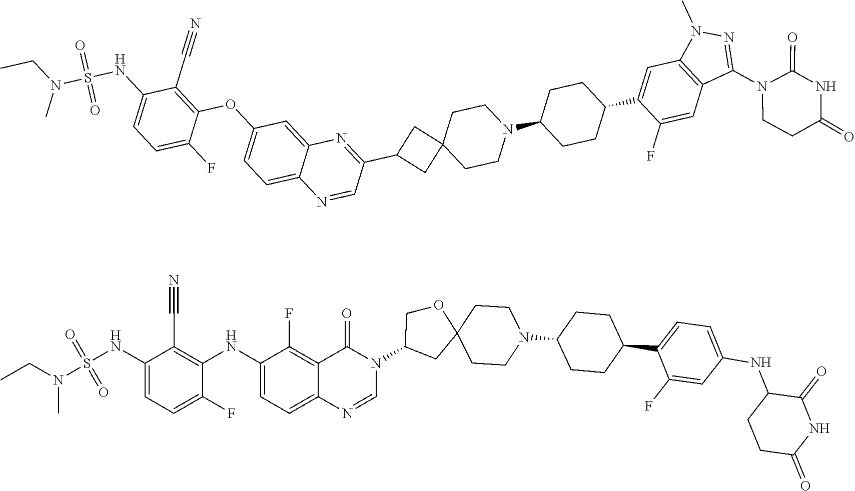 [SMILES: CCN(C)S(=O)(=O)Nc1ccc(F)c(Oc2ccc3ncc(C4CC5(CCN([C@H]6CC[C@H](c7cc8c(cc7F)c(N7CCC(=O)NC7=O)nn8C)CC6)CC5)C4)nc3c2)c1C#N ; CCN(C)S(=O)(=O)Nc1ccc(F)c(Nc2ccc3ncn([C@@H]4COC5(CCN([C@H]6CC[C@H](c7ccc(NC8CCC(=O)NC8=O)cc7F)CC6)CC5)C4)c(=O)c3c2F)c1C#N]

or a pharmaceutically acceptable salt thereof.

In another aspect a compound of the present invention has significant blood brain barrier penetration and can be used to treat a cancer that has metastasized to the brain. In certain aspects a method of treating a mutant BRAF mediated cancer that has metastasized to the brain or central nervous system (CNS) is provided comprising administering an effective amount of a compound of the present invention or a pharmaceutically acceptable salt thereof to a patient in need thereof. In certain embodiments the cancer that has metastasized to the brain or central nervous system is colorectal cancer, melanoma, or non-small cell lung cancer.

In certain aspects, a compound of the present invention is used to treat a BRAF mediated cancer, wherein the BRAF has mutated from the wild type. There are a number of possibilities for BRAF mutations. In certain non-limiting embodiments, the mutation is a Class I mutation, a Class II mutation, or a Class III mutation, or any combination thereof. Non-limiting examples of Class I mutations include V600 mutations such as V600E, V600K, V600R, V600D, V600M, and V600N. Non-limiting examples of Class II mutations include G469A, G469V, G469L, G469R, L597Q, and K601E. Non-limiting examples of Class III mutations include G466A, G466E, G466R, G466V, S467L, G469E, N581I, D594E, D594G, and D594N. In certain embodiments the BRAF mutation is a V600 mutation, for example V600E, and the cancer has metastasized to the brain or CNS.

In certain embodiments a compound of the present invention may treat a BRAF mutant mediated disorder wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E. In certain embodiments the BRAF mutation is not a Class I, Class II, or Class III mutation and the cancer has metastasized to the brain or CNS.

In certain embodiments a compound of the present invention treats a BRAF mutant mediated disorder wherein the mutation is a splice variant, for example p61-BRAF^{V600E} In certain embodiments the BRAF mutation is a splice variant, for example p61-BRAF^{V600E}, and the cancer has metastasized to the brain or CNS.

In certain embodiments a compound of the present invention is used to treat a disorder that is mediated by two or more mutant proteins, for example a cancer mediated by a BRAF^{V600E}/NRAS^{Q61K} double mutant. Non-limiting examples of double mutant cancers include colorectal cancer which is mediated by a BRAF mutation, for example BRAF^{V600E}, and a mutation of NRAS, MEK1, or PI3K, for example, BRAF^{V600E}/NRAS^{Q61R}, BRAF^{V600E}/MAP2K1^{P124S}, BRAF^{ZV600E}/PIK3CA^{H1047R} or BRAF^{ZV600E}/PIK3CA^{P449T}. In certain embodiments the cancer that is mediated by two or more mutant proteins has metastasized to the brain or CNS.

In certain embodiments, a compound of the present invention is used to treat a cancer that is resistant to at least one BRAF inhibitor, for example a cancer that is resistant to or has acquired resistance to a BRAF inhibitor selected from dabrafenib, vemurafenib, and encorafenib. In certain aspects the cancer that is resistant to treatment with a BRAF inhibitor has a RAF protein homo-dimerization or hetero-dimerization promoting mutation. For example, in certain embodiments a compound of the present invention is used to treat a cancer with one or more mutations that promote RAF dimerization. In certain embodiments the cancer with one or more RAF dimerization promoting mutations is resistant to treatment with a BRAF inhibitor for example dabrafenib, vemurafenib, or encorafenib. In certain aspects the mutation is a BRAF homo-dimerization promoting mutation. In certain embodiments a compound of the present invention is used to treat a BRAF mutant mediated cancer that has metastasized to the brain or CNS wherein the cancer has one or more RAF dimerization promoting mutations.

In certain embodiments a compound described herein is used to treat a cancer that has developed an escape mutation such as BRAF V600E/NRAS^{Q61K} double mutant cancer. In other embodiments, a compound described herein decreases phosphorylated ERK signal, indicating suppression of the EGFR-mediated MAPK pathway. In another embodiment, a compound described herein is used to treat a BRAF V600X colorectal cancer (CRC). In certain embodiments, a compound of the invention is used to treat BRAF-V600X non-small cell lung cancer (NSCLC). In certain embodiments a compound described herein shows activity against resistant tumor mutations such a point mutations in MEK, PI3K, splice variants such as p61-BRAF-V600E, BRAF kinase domain duplication, or a BRAF amplification.

In certain embodiments a compound described herein is used to treat melanoma. For example, in certain embodiments a compound of the present invention is used to treat a BRAF mutant driven cancer, such as melanoma that has metastasized to the brain or CNS. In certain embodiments a compound described herein is used to treat colorectal cancer. For example, in certain embodiments a compound of the present invention is used to treat a colorectal cancer that has metastasized to the brain or CNS. In certain embodiments a compound described herein is used to treat non-small cell lung cancer. For example, in certain embodiments a compound of the present invention is used to treat a non-small cell lung cancer that has metastasized to the brain or CNS.

In certain embodiments, a selected compound of the present invention provides an improved efficacy and/or safety profile relative to at least one known BRAF inhibitor. For example, a degrader of the present invention has the efficiency of an inhibitor only protein binding moiety combined with the catalytic degradation activity of the cereblon-activated proteasomal degradation. This provides rapid activity against the mutant BRAF mediated cancer by an active moiety that can quickly “return to action” and repeat the catalytic function. In this way, BRAF is quickly destroyed as done with a covalent suicide inhibitor, but without at the same time destroying the active drug.

In certain embodiments, the degrader compound of the present invention has one or more advantages in the treatment of a BRAF mediated disorder than using an enzyme inhibitor only.

In certain embodiments, less by mole of the compounds described herein is needed for the treatment of a BRAF mediated disorder, than by mole of the BRAF Targeting Ligand portion alone.

In certain embodiments, the compound of the present invention has less of at least one side-effect in the treatment of a BRAF mediated disorder, than by mole of the BRAF Targeting Ligand portion alone.

Another aspect of the present invention provides a compound as described herein, or an enantiomer, diastereomer, or stereoisomer thereof, or pharmaceutically acceptable salt, or a pharmaceutical composition, for use in the manufacture of a medicament for inhibiting or preventing a disorder mediated by BRAF or for modulating or decreasing the amount of BRAF.

Another aspect of the present invention provides a compound as described herein, or an enantiomer, diastereomer, or stereoisomer thereof, or pharmaceutically acceptable salt, or its pharmaceutical composition, for use in the manufacture of a medicament for treating or preventing a disease mediated by BRAF.

In certain embodiments, a selected compound as described herein is useful to treat a disorder comprising an abnormal cellular proliferation, such as a tumor or cancer, wherein BRAF is an oncogenic protein or a signaling mediator of the abnormal cellular proliferative pathway and its degradation decreases abnormal cell growth.

In certain embodiments, a compound of the present invention has at least one desired isotopic substitution of an atom, at an amount above the natural abundance of the isotope, i.e., enriched.

In certain embodiments, a compound of the present invention includes a deuterium atom or multiple deuterium atoms. In certain aspects the chiral center of the glutarimide is deuterated.

In certain embodiments a compound of the present invention is useful for the therapeutic treatment of cancer.

In certain aspects a compound of the present invention is used in combination with a second active agent described herein to treat a mutant BRAF mediated cancer. Non-limiting examples of classes of molecules that can be used in combination with a compound of the present invention include MEK inhibitors, immune checkpoint inhibitors, and EGFR antibodies. In certain embodiments a compound of the present invention is used in combination with trametinib for the treatment of a mutant BRAF mediated cancer, for example melanoma or non-small cell lung cancer. In certain embodiments a compound of the present invention is used in combination with an immune checkpoint inhibitor to treat a mutant BRAF mediated cancer. In certain embodiments a compound of the present invention is used in combination with cetuximab or panitumumab to treat a mutant BRAF mediated cancer, for example colorectal cancer. In certain embodiments a compound of the present invention is used in combination with nivolumab, pembrolizumab, cemiplimab, ipilimumab, relatlimab, atezolizumab, avelumab, or durvalumab to treat a mutant BRAF mediated cancer, for example colorectal cancer, melanoma, or non-small cell lung cancer.

In other aspects a compound of the present invention is used in combination with two or more additional active agents described herein to treat a mutant BRAF mediated cancer. In certain embodiments a compound described herein is used in combination with a MEK inhibitor and an immune checkpoint inhibitor to treat melanoma or non-small cell lung cancer.

Other features and advantages of the present application will be apparent from the following detailed description.

The present invention thus includes at least the following features
(a) A compound of the present invention or a pharmaceutically acceptable salt or isotopic derivative (including a deuterated derivative) thereof;
(b) A method for treating a mutant BRAF mediated disorder, such as an abnormal cellular proliferation, including cancer, comprising administering an effective amount of a compound of the present invention, or pharmaceutically acceptable salt thereof, as described herein, to a patient in need thereof;
(c) The method of (b) wherein the mutant BRAF mediated disorder is a cancer that has metastasized to the brain or CNS;
(d) A compound of the present invention, or a pharmaceutically acceptable salt, or isotopic derivative (including a deuterated derivative) thereof for use in the treatment of a disorder that is mediated by a mutant BRAF, for example an abnormal cellular proliferation such as a tumor or cancer;
(e) The compound of (d) wherein the mutant BRAF mediated disorder is a cancer that has metastasized to the brain or CNS;
(f) Use of a compound of the present invention, or a pharmaceutically acceptable salt thereof, in an effective amount in the treatment of a patient in need thereof, typically a human, with a mutant BRAF mediated disorder, for example an abnormal cellular proliferation such as a tumor or cancer;
(g) Use of a compound of the present invention, or a pharmaceutically acceptable salt or isotopic derivative (including a deuterated derivative) thereof in the manufacture of a medicament for the treatment of a mutant BRAF mediated disorder, for example an abnormal cellular proliferation such as a tumor or cancer;
(h) Use of a compound of the present invention, or a pharmaceutically acceptable salt thereof, in an effective amount in the treatment of a patient in need thereof, typically a human, with a mutant BRAF mediated disorder, for example an abnormal cellular proliferation such as a tumor or cancer;
(i) The use of any one of (f)-(h) wherein the mutant BRAF mediated disorder is a cancer that has metastasized to the brain or CNS;
(j) A pharmaceutical composition comprising an effective patient-treating amount of a compound of the present invention, or a pharmaceutically acceptable salt, isotopic derivative thereof; and a pharmaceutically acceptable carrier or diluent;
(k) A compound of the present invention, as described herein as a mixture of enantiomers or diastereomers (as relevant), including as a racemate;
(l) A compound of the present invention, as described herein in enantiomerically or diastereomerically (as relevant) enriched form, including an isolated enantiomer or diastereomer (i.e., about greater than 85, 90, 95, 97, or 99% pure); and
(m) A process for the preparation of therapeutic products that contain an effective amount of a compound of the present invention, or a pharmaceutically acceptable salt thereof, as described herein.

### BRIEF DESCRIPTION OF THE DRAWINGS

FIG. 1 is a line graph showing body weight changes after administration of vehicle (10 μL/g, p.o., BID), encorafenib (35 mg/kg, p.o., QD), dabrafenib (100 mg/kg, p.o., QD), Compound 83 (10 mg/kg, p.o., BID), Compound 93 (10 mg/kg, p.o., BID), Compound 70 (10 mg/kg, p.o., BID), or Compound 54 (10 mg/kg, p.o., BID) by oral gavage (p.o.) to female BALB/c nude mice bearing A375 xenografts. Data points represent group mean body weight. Error bars represent standard error of the mean (SEM). The x-axis is the time measured in days and the y-axis is body weight change in grams. The experiment was conducted as described in Example 6.

FIG. 2 is a line graph showing percent body weight (BW) Change after administration of vehicle (10 μL/g, p.o., BID), encorafenib (35 mg/kg, p.o., QD), dabrafenib (100 mg/kg, p.o., QD), Compound 83 (10 mg/kg, p.o., BID), Compound 93 (10 mg/kg, p.o., BID), Compound 70 (10 mg/kg, p.o., BID), or Compound 54 (10 mg/kg, p.o., BID) by oral gavage (p.o.) to female BALB/c nude mice bearing A375 xenografts. BW change was calculated based on animal weight on the first day of grouping. Data points represent percent group mean change in BW. Error bars represent standard error of the mean (SEM). The x-axis is the time measured in days and the y-axis is body weight change in percentage. The experiment was conducted as described in Example 6.

FIG. 3 is a line graph showing in vivo efficacy of encorafenib, dabrafenib, Compound 83, Compound 93, Compound 70, and Compound 54 in the treatment of female BALB/c nude mice bearing A375 tumors. Mice were treated with vehicle (10 μL/g, p.o., BID), encorafenib (35 mg/kg, p.o., QD), dabrafenib (100 mg/kg, p.o., QD), Compound 83 (10 mg/kg, p.o., BID), Compound 93 (10 mg/kg, p.o., BID), Compound 70 (10 mg/kg, p.o., BID), or Compound 54 (10 mg/kg, p.o., BID) by oral gavage (p.o.). Data points represent group mean change in tumor volume. Error bars represent standard error of the mean (SEM). The x-axis is the time measured in days and the y-axis is A375 tumor volume measured in mm³. The experiment was conducted as described in Example 6.

FIG. 4 is a bar graph providing the concentration of encorafenib, dabrafenib, Compound 83, Compound 93, Compound 70, and Compound 54 in plasma and liver following oral dosing (p.o.) of encorafenib (35 mg/kg), dabrafenib (100 mg/kg), Compound 83 (10 mg/kg), Compound 93 (10 mg/kg), Compound 70 (10 mg/kg), or Compound 54 (10 mg/kg). The samples were collected 6 hours after the last dose treatment of female BALB/c nude mice bearing A375 tumors. The experiment was conducted as described in Example 6.

FIG. 5 is a bar graph providing the concentration of encorafenib in plasma, tumor and liver following a single oral (PO) dose at 35 mg/kg. Plasma, tumor and liver samples were harvested at the indicated timepoints following treatment of female BALB/c nude mice bearing A375 tumors for analysis. The experiment was conducted as described in Example 6.

FIG. 6 is a bar graph providing the concentration of dabrafenib in plasma, tumor and liver following a single oral (PO) dose at 100 mg/kg. Plasma, tumor and liver samples were harvested at the indicated timepoints following treatment of female BALB/c nude mice bearing A375 tumors for analysis. The experiment was conducted as described in Example 6.

FIG. 7 is a bar graph providing the concentration of Compound 83 in plasma, tumor and liver following a single oral (PO) dose at 10 mg/kg. Plasma, tumor and liver samples were harvested at the indicated timepoints following treatment of female BALB/c nude mice bearing A375 tumors for analysis. The experiment was conducted as described in Example 6.

FIG. 8 is a bar graph providing the concentration of Compound 93 in plasma, tumor and liver following a single oral (PO) dose at 10 mg/kg. Plasma, tumor and liver samples were harvested at the indicated timepoints following treatment of female BALB/c nude mice bearing A375 tumors for analysis. The experiment was conducted as described in Example 6.

FIG. 9 is a bar graph providing the concentration of Compound 70 in plasma, tumor and liver following a single oral (PO) dose at 10 mg/kg. Plasma, tumor and liver samples were harvested at the indicated timepoints following treatment of female BALB/c nude mice bearing A375 tumors for analysis. The experiment was conducted as described in Example 6.

FIG. 10 is a bar graph providing the concentration of Compound 54 in plasma, tumor and liver following a single oral (PO) dose at 10 mg/kg. Plasma, tumor and liver samples were harvested at the indicated timepoints following treatment of female BALB/c nude mice bearing A375 tumors for analysis. The experiment was conducted as described in Example 6.

FIG. 11A-11F are Western blots and matching bar graphs of BRAF and phosphorylated ERK(p-ERK) expression level in A375 tumor samples, an end point measurement for the activation of the mitogen-activated protein kinase (MAPK) signaling pathway, at multiple timepoints after administration of vehicle, encorafenib (35 mg/kg), dabrafenib (100 mg/kg), Compound 83 (10 mg/kg), Compound 93 (10 mg/kg), Compound 70 (10 mg/kg), or Compound 54 (10 mg/kg). The experiment was conducted as described in Example 6.

FIG. 12 is a line graph showing body weight changes after administration of vehicle (10 μL/g, p.o., BID), dabrafenib (100 mg/kg, p.o., QD), Compound 141 (10 mg/kg, p.o., QD), Compound 141 (10 mg/kg, p.o., BID), Compound 118 (10 mg/kg, p.o., BID), Compound 156 (10 mg/kg, p.o., BID), or Compound 180 (10 mg/kg, p.o., BID) by oral gavage (p.o.) to female BALB/c nude mice bearing A375 xenografts. Data points represent group mean body weight. Error bars represent standard error of the mean (SEM). The x-axis is the time measured in days and the y-axis is body weight change in grams. The experiment was conducted as described in Example 7.

FIG. 13 is a line graph showing percent body weight (BW) Change after administration of vehicle (10 μL/g, p.o., BID), dabrafenib (100 mg/kg, p.o., QD), Compound 141 (10 mg/kg, p.o., QD), Compound 141 (10 mg/kg, p.o., BID), Compound 118 (10 mg/kg, p.o., BID), Compound 156 (10 mg/kg, p.o., BID), or Compound 180 (10 mg/kg, p.o., BID) by oral gavage (p.o.) to female BALB/c nude mice bearing A375 xenografts. BW change was calculated based on animal weight on the first day of grouping. Data points represent percent group mean change in BW. Error bars represent standard error of the mean (SEM). The x-axis is the time measured in days and the y-axis is body weight change in percentage. The experiment was conducted as described in Example 7.

FIG. 14 is a line graph showing in vivo efficacy of dabrafenib, Compound 141, Compound 118, Compound 156, and Compound 180 in the treatment of female BALB/c nude mice bearing A375 tumors. Mice were treated with vehicle (10 μL/g, p.o., BID), dabrafenib (100 mg/kg, p.o., QD), Compound 141 (10 mg/kg, p.o., QD), Compound 141 (10 mg/kg, p.o., BID), Compound 118 (10 mg/kg, p.o., BID), Compound 156 (10 mg/kg, p.o., BID), or Compound 180 (10 mg/kg, p.o., BID) by oral gavage (p.o.). Data points represent group mean change in tumor volume. Error bars represent standard error of the mean (SEM). The x-axis is the time measured in days and the y-axis is A375 tumor volume measured in mm³. The experiment was conducted as described in Example 7.

FIG. 15 is a bar graph providing the concentration of dabrafenib, Compound 141, Compound 118, Compound 156, and Compound 180 in plasma and liver following oral dosing (p.o.) of dabrafenib (100 mg/kg, p.o., QD), Compound 141 (10 mg/kg, p.o., QD), Compound 141 (10 mg/kg, p.o., BID), Compound 118 (10 mg/kg, p.o., BID), Compound 156 (10 mg/kg, p.o., BID), or Compound 180 (10 mg/kg, p.o., BID). The samples were collected 6 hours after the last dose treatment of female BALB/c nude mice bearing A375 tumors. The experiment was conducted as described in Example 7.

FIG. 16 is a bar graph providing the concentration of dabrafenib, Compound 141, Compound 118, Compound 156, and Compound 180 in plasma following a single oral (PO) dose at 100 mg/kg for dabrafenib and 10 mg/kg for the rest of the test compounds. Plasma samples were collected at 1 hour, 6 hours, 12 hours, and 24 hours after treatment of female BALB/c nude mice bearing A375 tumors for analysis. The experiment was conducted as described in Example 7.

FIG. 17 is a bar graph providing the concentration of dabrafenib, Compound 141, Compound 118, Compound 156, and Compound 180 in tumor following a single oral (PO) dose at 100 mg/kg for dabrafenib and 10 mg/kg for the rest of the test compounds. Tumor samples were collected at 1 hour, 6 hours, 12 hours, and 24 hours after treatment of female BALB/c nude mice bearing A375 tumors for analysis. The experiment was conducted as described in Example 7.

FIG. 18 s a bar graph providing the concentration of dabrafenib, Compound 141, Compound 118, Compound 156, and Compound 180 in liver following a single oral (PO) dose at 100 mg/kg for dabrafenib and 10 mg/kg for the rest of the test compounds. Liver samples were collected at 6 hours and 12 hours after treatment of female BALB/c nude mice bearing A375 tumors for analysis. The experiment was conducted as described in Example 7.

FIG. 19A-19E are Western blots and matching bar graphs of BRAF and phosphorylated ERK(p-ERK) expression level in A375 tumor samples after administration of vehicle, dabrafenib (100 mg/kg), Compound 141 (10 mg/kg), Compound 118 (10 mg/kg), Compound 156 (10 mg/kg), or Compound 180 (10 mg/kg). The experiment was conducted as described in Example 7.

FIG. 20 is a line graph showing body weight changes after administration of vehicle (10 μL/g, p.o., BID), encorafenib (35 mg/kg, p.o., QD), Compound 8 (10 mg/kg, p.o., BID), Compound 26 (10 mg/kg, p.o., BID), or Compound 38 (10 mg/kg, p.o., BID) by oral gavage (p.o.) to female BALB/c nude mice bearing A375 xenografts. Data points represent group mean body weight. Error bars represent standard error of the mean (SEM). The x-axis is the time measured in days and the y-axis is body weight change in grams. The experiment was conducted as described in Example 8.

FIG. 21 is a line graph showing percent body weight (BW) Change after administration of vehicle (10 μL/g, p.o., BID), encorafenib (35 mg/kg, p.o., QD), Compound 8 (10 mg/kg, p.o., BID), Compound 26 (10 mg/kg, p.o., BID), or Compound 38 (10 mg/kg, p.o., BID) by oral gavage (p.o.) to female BALB/c nude mice bearing A375 xenografts. BW change was calculated based on animal weight on the first day of grouping. Data points represent percent group mean change in BW. Error bars represent standard error of the mean (SEM). The x-axis is the time measured in days and the y-axis is body weight change in percentage. The experiment was conducted as described in Example 8.

FIG. 22 is a line graph showing in vivo efficacy of encorafenib, Compound 8, Compound 26, and Compound 38 in the treatment of female BALB/c nude mice bearing A375 tumors. Mice were treated with vehicle (10 μL/g, p.o., BID), encorafenib (35 mg/kg, p.o., QD), Compound 8 (10 mg/kg, p.o., BID), Compound 26 (10 mg/kg, p.o., BID), or Compound 38 (10 mg/kg, p.o., BID) by oral gavage (p.o.). Data points represent group mean change in tumor volume. Error bars represent standard error of the mean (SEM). The x-axis is the time measured in days and the y-axis is A375 tumor volume measured in mm³. The experiment was conducted as described in Example 8.

FIG. 23 is a bar graph providing the concentration of encorafenib, Compound 8, Compound 26, and Compound 38 in plasma and liver following oral dosing (p.o.) of encorafenib (35 mg/kg, p.o., QD), Compound 8 (10 mg/kg, p.o., BID), Compound 26 (10 mg/kg, p.o., BID), or Compound 38 (10 mg/kg, p.o., BID). The samples were collected 6 hours after the last dose treatment of female BALB/c nude mice bearing A375 tumors\The experiment was conducted as described in Example 8.

FIG. 24 is a bar graph providing the concentration of encorafenib, Compound 8, Compound 26, Compound 38, and Compound 39 in plasma following a single oral (PO) dose at 35 mg/kg for encorafenib and 10 mg/kg for the rest of the test compounds. Plasma samples were collected at 1 hour, 6 hours, 12 hours, and 24 hours after treatment of female BALB/c nude mice bearing A375 tumors for analysis. \The experiment was conducted as described in Example 8.

FIG. 25 is a bar graph providing the concentration of encorafenib, Compound 8, Compound 26, Compound 38, and Compound 39 in tumor following a single oral (PO) dose at 35 mg/kg for encorafenib and 10 mg/kg for the rest of the test compounds. Tumor samples were collected at 1 hour, 6 hours, 12 hours, and 24 hours after treatment of female BALB/c nude mice bearing A375 tumors for analysis. The experiment was conducted as described in Example 8.

FIG. 26 is a bar graph providing the concentration of encorafenib, Compound 8, Compound 26, Compound 38, and Compound 39 in liver following a single oral (PO) dose at 35 mg/kg for encorafenib and 10 mg/kg for the rest of the test compounds. Liver samples were collected at 6 hours and 12 hours after treatment of female BALB/c nude mice bearing A375 tumors for analysis. The experiment was conducted as described in Example 8.

FIG. 27A-27E are Western blots and matching bar graphs of BRAF and p-ERK expression level in A375 tumor samples after administration of vehicle, encorafenib, Compound 8, Compound 26, Compound 38, and Compound 39. The experiment was conducted as described in Example 8.

FIG. 28 is a line graph showing body weight changes after administration of vehicle (10 μL/g, p.o., BID), dabrafenib (100 mg/kg, p.o., QD), Compound 218 (10 mg/kg, p.o., BID), or Compound 157 (10 mg/kg, p.o., BID) by oral gavage (p.o.) to female BALB/c nude mice bearing A375 xenografts. Data points represent group mean body weight. Error bars represent standard error of the mean (SEM). The x-axis is the time measured in days and the y-axis is body weight change in grams. The experiment was conducted as described in Example 9.

FIG. 29 is a line graph showing percent body weight (BW) Change after administration of vehicle (10 μL/g, p.o., BID), dabrafenib (100 mg/kg, p.o., QD), Compound 218 (10 mg/kg, p.o., BID), or Compound 157 (10 mg/kg, p.o., BID) by oral gavage (p.o.) to female BALB/c nude mice bearing A375 xenografts. BW change was calculated based on animal weight on the first day of grouping. Data points represent percent group mean change in BW. Error bars represent standard error of the mean (SEM). The x-axis is the time measured in days and the y-axis is body weight change in percentage. The experiment was conducted as described in Example 9.

FIG. 30 is a line graph showing in vivo efficacy of dabrafenib, Compound 218, and Compound 157 in the treatment of female BALB/c nude mice bearing A375 tumors. Mice were treated with vehicle (10 μL/g, p.o., BID), dabrafenib (100 mg/kg, p.o., QD), Compound 218 (10 mg/kg, p.o., BID), or Compound 157 (10 mg/kg, p.o., BID) by oral gavage (p.o.). Data points represent group mean change in tumor volume. Error bars represent standard error of the mean (SEM). The x-axis is the time measured in days and the y-axis is A375 tumor volume measured in mm³. The experiment was conducted as described in Example 9.

FIG. 31 is a bar graph providing the concentration of dabrafenib, Compound 218, and Compound 157 in plasma and liver following oral dosing (p.o.) of dabrafenib (100 mg/kg, p.o., QD), Compound 218 (10 mg/kg, p.o., BID), or Compound 157 (10 mg/kg, p.o., BID). The samples were collected 6 hours after the last dose treatment of female BALB/c nude mice bearing A375 tumors. The experiment was conducted as described in Example 9.

FIG. 32 is a line graph showing body weight changes after administration of vehicle (10 μL/g, p.o., BID), Compound 133 (10 mg/kg, p.o., BID), Compound 144 (10 mg/kg, p.o., BID), or Compound 198 (10 mg/kg, p.o., BID) by oral gavage (p.o.) to female BALB/c nude mice bearing A375 xenografts. Data points represent group mean body weight. Error bars represent standard error of the mean (SEM). The x-axis is the time measured in days and the y-axis is body weight change in grams. The experiment was conducted as described in Example 9.

FIG. 33 is a line graph showing percent body weight (BW) Change after administration of vehicle (10 μL/g, p.o., BID), Compound 133 (10 mg/kg, p.o., BID), Compound 144 (10 mg/kg, p.o., BID), or Compound 198 (10 mg/kg, p.o., BID) by oral gavage (p.o.) to female BALB/c nude mice bearing A375 xenografts. BW change was calculated based on animal weight on the first day of grouping. Data points represent percent group mean change in BW. Error bars represent standard error of the mean (SEM). The x-axis is the time measured in days and the y-axis is body weight change in percentage. The experiment was conducted as described in Example 9.

FIG. 34 is a line graph showing in vivo efficacy of Compound 133, Compound 144, and Compound 198 in the treatment of female BALB/c nude mice bearing A375 tumors. Mice were treated with vehicle (10 μL/g, p.o., BID), Compound 133 (10 mg/kg, p.o., BID), Compound 144 (10 mg/kg, p.o., BID), or Compound 198 (10 mg/kg, p.o., BID) by oral gavage (p.o.). Data points represent group mean change in tumor volume. Error bars represent standard error of the mean (SEM). The x-axis is the time measured in days and the y-axis is A375 tumor volume measured in mm³. The experiment was conducted as described in Example 9.

FIG. 35 is a bar graph providing the concentration of Compound 133, Compound 144, and Compound 198 in plasma and liver following oral dosing (p.o.) of Compound 133 (10 mg/kg, p.o., BID), Compound 144 (10 mg/kg, p.o., BID), or Compound 198 (10 mg/kg, p.o., BID). The samples were collected 6 hours after the last dose treatment of female BALB/c nude mice bearing A375 tumors. The experiment was conducted as described in Example 9.

FIG. 36 is a bar graph providing the concentration of dabrafenib, Compound 218, and Compound 157 in plasma following a single oral (PO) dose at 100 mg/kg for dabrafenib and 10 mg/kg for the rest of the test compounds. Plasma samples were collected at 1 hour, 6 hours, 12 hours, and 24 hours after treatment of female BALB/c nude mice bearing A375 tumors for analysis. The experiment was conducted as described in Example 9.

FIG. 37 is a bar graph providing the concentration of dabrafenib, Compound 218, and Compound 157 in tumor following a single oral (PO) dose at 100 mg/kg for dabrafenib and 10 mg/kg for the rest of the test compounds. Tumor samples were collected at 1 hour, 6 hours, 12 hours, and 24 hours after treatment of female BALB/c nude mice bearing A375 tumors for analysis. The experiment was conducted as described in Example 9.

FIG. 38 is a bar graph providing the concentration of dabrafenib, Compound 218, and Compound 157 in plasma following a single oral (PO) dose at 100 mg/kg for dabrafenib and 10 mg/kg for the rest of the test compounds. Plasma samples were collected at 6 hours, and 12 hours after treatment of female BALB/c nude mice bearing A375 tumors for analysis. The experiment was conducted as described in Example 9.

FIG. 39A-39C are western blots and matching bar graphs showing the Western Blotting analysis of BRAF and p-ERK expression level in A375 tumor samples after administration of vehicle, dabrafenib, Compound 218, and Compound 157. The experiment was conducted as described in Example 9.

FIG. 40 is a bar graph showing the concentration of Compound 133, Compound 144, Compound 198 in plasma samples collected at 1 hour, 6 hours, 12 hours, and 24 hours after treatment of female BALB/c nude mice bearing A375 tumors. The experiment was conducted as described in Example 9.

FIG. 41 is a bar graph showing the concentration of Compound 133, Compound 144, Compound 198 in tumor samples collected at 1 hour, 6 hours, 12 hours, and 24 hours after treatment of female BALB/c nude mice bearing A375 tumors. The experiment was conducted as described in Example 9.

FIG. 42 is a bar graph showing the concentration of Compound 133, Compound 144, Compound 198 in liver samples collected at 6 and 12 hours post administration. The experiment was conducted as described in Example 9.

FIG. 43A-43C are western blots and matching bar graphs showing the Western Blotting analysis of BRAF and p-ERK expression level in A375 tumor samples after administration of vehicle, Compound 133, Compound 144, Compound 198. The experiment was conducted as described in Example 9.

FIG. 44 is a bar graph showing the concentration of Compound 204 in plasma, tumor and liver samples collected at 1 hour, 6 hours, 12 hours, and 24 hours after treatment of female BALB/c nude mice bearing A375 tumors. The experiment was conducted as described in Example 9.

FIG. 45 is a bar graph and matching western blot showing the Western Blotting analysis of BRAF and p-ERK expression level in A375 tumor samples after administration of vehicle and Compound 204. The experiment was conducted as described in Example 9.

FIG. 46 is a plot showing the HT29 human colorectal adenocarcinoma cell line percent viability after administration of various concentrations of Compound 26 and Compound 141 in vitro. The x-axis is concentration measured in nM and the y-axis is percent viability. The experiment was conducted as described in Example 11.

FIG. 47 is a plot of bioluminescence signal change over time after administration of vehicle, encorafenib, Compound 26, Compound 141 in a luciferase-tagged A375 human melanoma CNS tumor study. The x-axis is days after start of treatment and the y-axis is bioluminescence signal change. The experiment was conducted as described in Example 12.

FIG. 48 is the survival curve for the mice after administration of vehicle, encorafenib, Compound 26, Compound 141 in CNS tumor study. The x-axis is time measured in days and the y-axis is probability of survival. The experiment was conducted as described in Example 12.

FIG. 49 is a plot showing the concentration of Compound 26, Compound 141, and encorafenib found in the plasma in the luciferase-tagged A375 CNS xenograft pharmacokinetics (PK) study. The x-axis is time of treatment measured in hours and the y-axis is concentration in the plasma measured in ng/mL. The experiment was conducted as described in Example 12.

FIG. 50 is a plot showing the concentration of Compound 26, Compound 141, and encorafenib found in the brain tissue adjacent to the tumor in the luciferase-tagged A375 CNS xenograft pharmacokinetics (PK) study. The x-axis is time after treatment measured in hours and the y-axis is concentration in the brain tissue measured in ng/mL. The experiment was conducted as described in Example 12.

FIG. 51 is a plot showing the pharmacodynamics of BRAF protein degradation in the inoculated CNS tumors. The x-axis is time after treatment measured in hours and the y-axis is concentration of BRAF^{V600E} in the brain tumor displayed as a normalized ratio. The experiment was conducted as described in Example 12.

FIG. 52 is a bar graph providing the concentration of dabrafenib in plasma following an oral (PO) dose at 100 mg/kg, QD. Plasma samples were harvested at the indicated timepoints following treatment of female BALB/c nude mice bearing A375 tumors for analysis. The experiment was conducted as described in Example 13.

FIG. 53 is a bar graph providing the concentration of dabrafenib in tumor and brain following an oral (PO) dose at 100 mg/kg, QD. Tumor and brain samples were harvested at the indicated timepoints following treatment of female BALB/c nude mice bearing A375 tumors for analysis. The experiment was conducted as described in Example 13.

FIG. 54 is a bar graph providing the concentration of Compound 26 in plasma following an oral (PO) dose at 1.5 mg/kg BID. Plasma samples were harvested at the indicated timepoints following treatment of female BALB/c nude mice bearing A375 tumors for analysis. The experiment was conducted as described in Example 13.

FIG. 55 is a bar graph providing the concentration of Compound 26 in tumor and brain following an oral (PO) dose at 1.5 mg/kg BID. Tumor and brain samples were harvested at the indicated timepoints following treatment of female BALB/c nude mice bearing A375 tumors for analysis. The experiment was conducted as described in Example 13.

FIG. 56 is a bar graph providing the concentration of Compound 26 in plasma following an oral (PO) dose at 2 mg/kg TID. Plasma samples were harvested at the indicated timepoints following treatment of female BALB/c nude mice bearing A375 tumors for analysis. The experiment was conducted as described in Example 13.

FIG. 57 is a bar graph providing the concentration of Compound 26 in tumor and brain following an oral (PO) dose at 2 mg/kg TID. Tumor and brain samples were harvested at the indicated timepoints following treatment of female BALB/c nude mice bearing A375 tumors for analysis. The experiment was conducted as described in Example 13.

FIG. 58 is a bar graph providing the concentration of Compound 26 in plasma following an oral (PO) dose at 3 mg/kg BID. Plasma samples were harvested at the indicated timepoints following treatment of female BALB/c nude mice bearing A375 tumors for analysis. The experiment was conducted as described in Example 13.

FIG. 59 is a bar graph providing the concentration of Compound 26 in tumor and brain following an oral (PO) dose at 3 mg/kg BID. Tumor and brain samples were harvested at the indicated timepoints following treatment of female BALB/c nude mice bearing A375 tumors for analysis. The experiment was conducted as described in Example 13.

FIG. 60 is a bar graph providing the concentration of Compound 26 in plasma following an oral (PO) dose at 6 mg/kg QD. Plasma samples were harvested at the indicated timepoints following treatment of female BALB/c nude mice bearing A375 tumors for analysis. The experiment was conducted as described in Example 13.

FIG. 61 is a bar graph providing the concentration of Compound 26 in tumor and brain following an oral (PO) dose at 6 mg/kg QD. Tumor and brain samples were harvested at the indicated timepoints following treatment of female BALB/c nude mice bearing A375 tumors for analysis. The experiment was conducted as described in Example 13.

FIG. 62 is a bar graph providing the concentration of Compound 26 in plasma following an oral (PO) dose at 10 mg/kg BID. Plasma samples were harvested at the indicated timepoints following treatment of female BALB/c nude mice bearing A375 tumors for analysis. The experiment was conducted as described in Example 13.

FIG. 63 is a bar graph providing the concentration of Compound 26 in tumor and brain following an oral (PO) dose at 10 mg/kg BID. Tumor and brain samples were harvested at the indicated timepoints following treatment of female BALB/c nude mice bearing A375 tumors for analysis. The experiment was conducted as described in Example 13.

FIG. 64 is a bar graph providing the concentration of Compound 141 in plasma following an oral (PO) dose at 0.75 mg/kg BID. Plasma samples were harvested at the indicated timepoints following treatment of female BALB/c nude mice bearing A375 tumors for analysis. The experiment was conducted as described in Example 13.

FIG. 65 is a bar graph providing the concentration of Compound 141 in tumor and brain following an oral (PO) dose at 0.75 mg/kg BID. Tumor and brain samples were harvested at the indicated timepoints following treatment of female BALB/c nude mice bearing A375 tumors for analysis. The experiment was conducted as described in Example 13.

FIG. 66 is a bar graph providing the concentration of Compound 141 in plasma following an oral (PO) dose at 1 mg/kg TID. Plasma samples were harvested at the indicated timepoints following treatment of female BALB/c nude mice bearing A375 tumors for analysis. The experiment was conducted as described in Example 13.

FIG. 67 is a bar graph providing the concentration of Compound 141 in tumor and brain following an oral (PO) dose at 1 mg/kg TID. Tumor and brain samples were harvested at the indicated timepoints following treatment of female BALB/c nude mice bearing A375 tumors for analysis. The experiment was conducted as described in Example 13.

FIG. 68 is a bar graph providing the concentration of Compound 141 in plasma following an oral (PO) dose at 1.5 mg/kg BID. Plasma samples were harvested at the indicated timepoints following treatment of female BALB/c nude mice bearing A375 tumors for analysis. The experiment was conducted as described in Example 13.

FIG. 69 is a bar graph providing the concentration of Compound 141 in tumor and brain following an oral (PO) dose at 1.5 mg/kg BID. Tumor and brain samples were harvested at the indicated timepoints following treatment of female BALB/c nude mice bearing A375 tumors for analysis. The experiment was conducted as described in Example 13.

FIG. 70 is a bar graph providing the concentration of Compound 141 in plasma following an oral (PO) dose at 3 mg/kg QD. Plasma samples were harvested at the indicated timepoints following treatment of female BALB/c nude mice bearing A375 tumors for analysis. The experiment was conducted as described in Example 13.

FIG. 71 s a bar graph providing the concentration of Compound 141 in tumor and brain following an oral (PO) dose at 3 mg/kg QD. Tumor and brain samples were harvested at the indicated timepoints following treatment of female BALB/c nude mice bearing A375 tumors for analysis. The experiment was conducted as described in Example 13.

FIG. 72 is a bar graph providing the concentration of Compound 141 in plasma following an oral (PO) dose at 5 mg/kg BID. Plasma samples were harvested at the indicated timepoints following treatment of female BALB/c nude mice bearing A375 tumors for analysis. The experiment was conducted as described in Example 13.

FIG. 73 is a bar graph providing the concentration of Compound 141 in tumor and brain following an oral (PO) dose at 5 mg/kg BID. Tumor and brain samples were harvested at the indicated timepoints following treatment of female BALB/c nude mice bearing A375 tumors for analysis. The experiment was conducted as described in Example 13.

FIG. 74A-74M are western blots and matching bar graphs showing the Western Blotting analysis of BRAF and p-ERK expression level in A375 tumor samples after administration of vehicle, dabrafenib, Compound 26 or Compound 141. The experiment was conducted as described in Example 13.

FIG. 75 is a line graph of body weight (BW) changes after administration of Compound 26, Compound 141 or dabrafenib to female BALB/c nude mice bearing A375 xenografts. Data points represent group mean body weight. Error bars represent standard error of the mean (SEM). The experiment was conducted as described in Example 14.

FIG. 76 is a line graph of percent body weight changes after administration of Compound 26, Compound 141 or dabrafenib to female BALB/c nude mice bearing A375 xenografts. BW change was calculated based on animal weight on the first day of grouping. Data points represent group mean body weight. Error bars represent standard error of the mean (SEM). The experiment was conducted as described in Example 14.

FIG. 77 is a line graph of tumor volume trace after administration of Compound 26, Compound 141 and dabrafenib to female BALB/c nude mice bearing A375 xenografts. Data points represent percent group mean tumor volume. Error bars represent standard error of the mean (SEM). The experiment was conducted as described in Example 14.

FIG. 78 is a bar graph providing the concentration of dabrafenib in plasma, tumor and brain following an oral (PO) dose at 100 mg/kg QD. Plasma, tumor and brain samples were harvested at the indicated timepoints following treatment of female BALB/c nude mice bearing A375 tumors for analysis. The experiment was conducted as described in Example 14.

FIG. 79 is a bar graph providing the concentration of Compound 26 in plasma following different oral (PO) doses. Plasma samples were harvested at the indicated timepoints following treatment of female BALB/c nude mice bearing A375 tumors for analysis. The experiment was conducted as described in Example 14.

FIG. 80 is a bar graph providing the concentration of Compound 26 in tumor and brain following different oral (PO) doses. Tumor and brain samples were harvested at the indicated timepoints following treatment of female BALB/c nude mice bearing A375 tumors for analysis. The experiment was conducted as described in Example 14.

FIG. 81 is a bar graph providing the concentration of Compound 141 in plasma following different oral (PO) doses. Plasma samples were harvested at the indicated timepoints following treatment of female BALB/c nude mice bearing A375 tumors for analysis. The experiment was conducted as described in Example 14.

FIG. 82 is a bar graph providing the concentration of Compound 141 in tumor and brain following different oral (PO) doses. Tumor and brain samples were harvested at the indicated timepoints following treatment of female BALB/c nude mice bearing A375 tumors for analysis. The experiment was conducted as described in Example 14.

FIG. 83 is a line graph showing the results of Compound 26, Compound 26-NMe, Compound 141, and Compound 141-NMe in an A375 viability assay. The y-axis is viability in % and the x-axis is concentration (nM). The experiment was conducted as described in Example 15.

FIG. 84 is a line graph showing the result of a cell viability assay in V600K cell line (CTGlo) for Compound 26 and Compound 141. The y-axis is viability in % and the x-axis is concentration (Log(nM)). The experiment was conducted as described in Example 16.

FIG. 85 is an extended survival curve for the BRAF intracranial model experiment after administration of vehicle, encorafenib, Compound 26, and Compound 141 in the CNS tumor study. The x-axis is time measured in days and the y-axis is probability of survival. The experiment was conducted as described in Example 12.

### DETAILED DESCRIPTION

In certain embodiments, the present invention provides a compound of Formula I to Formula XXXII:

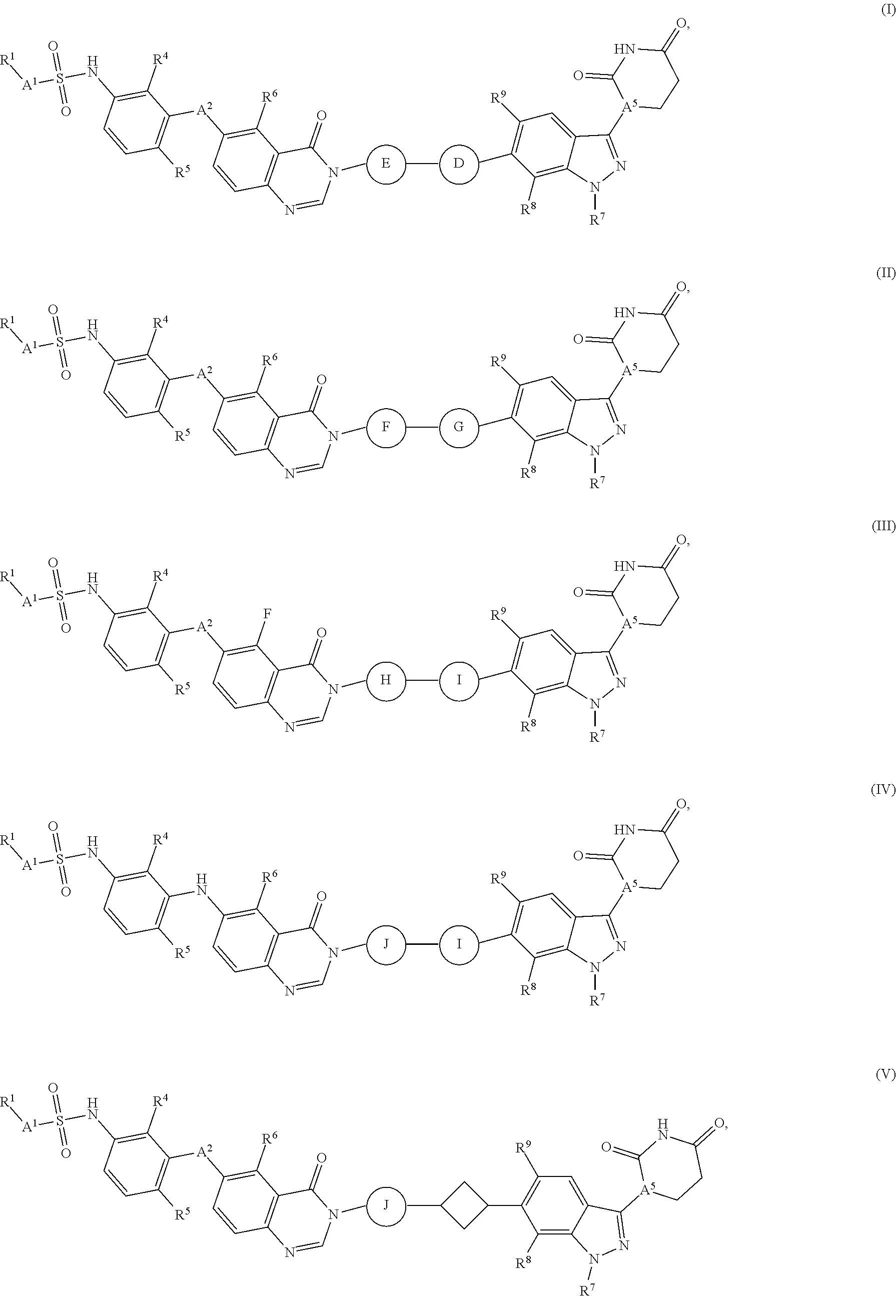

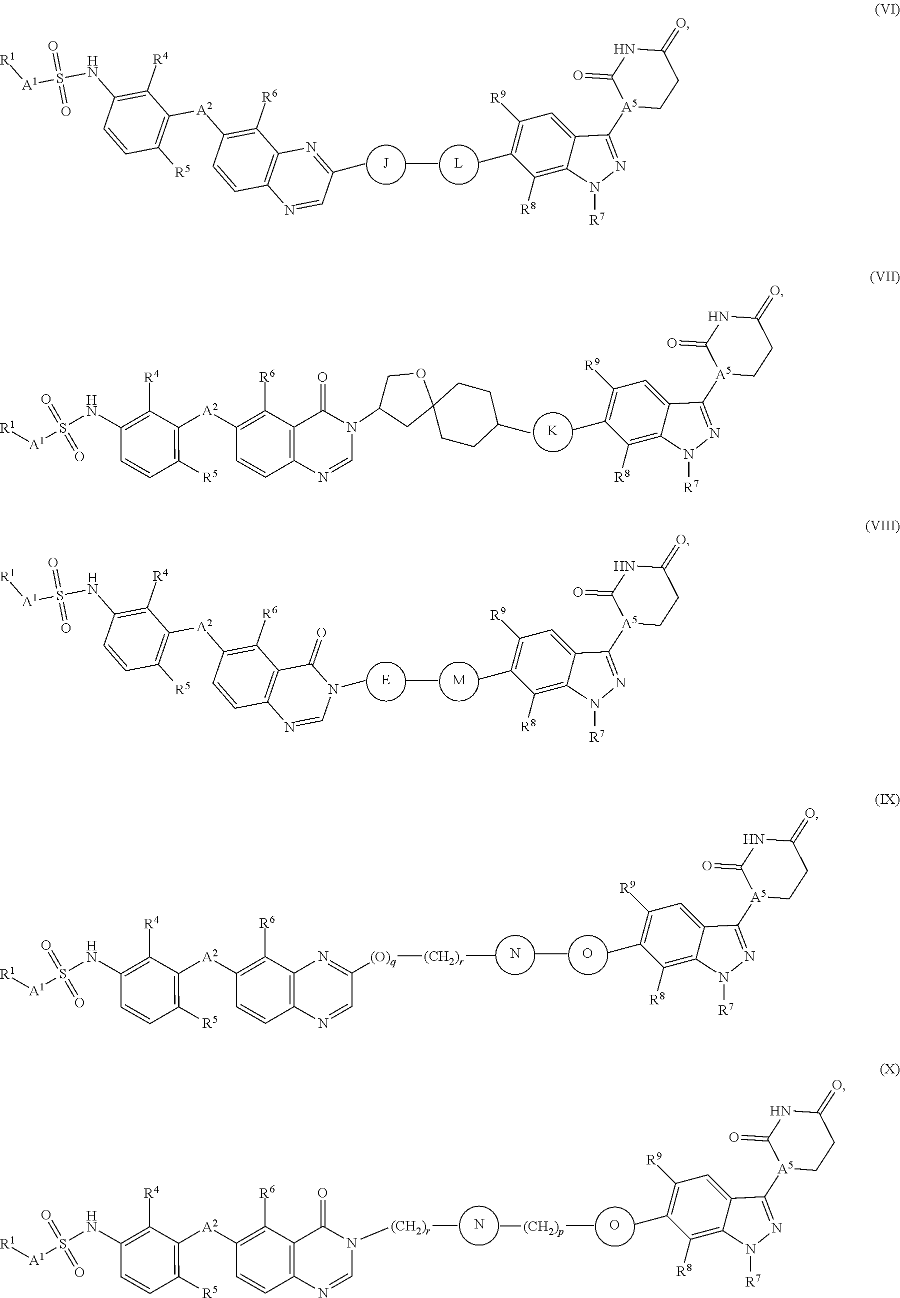

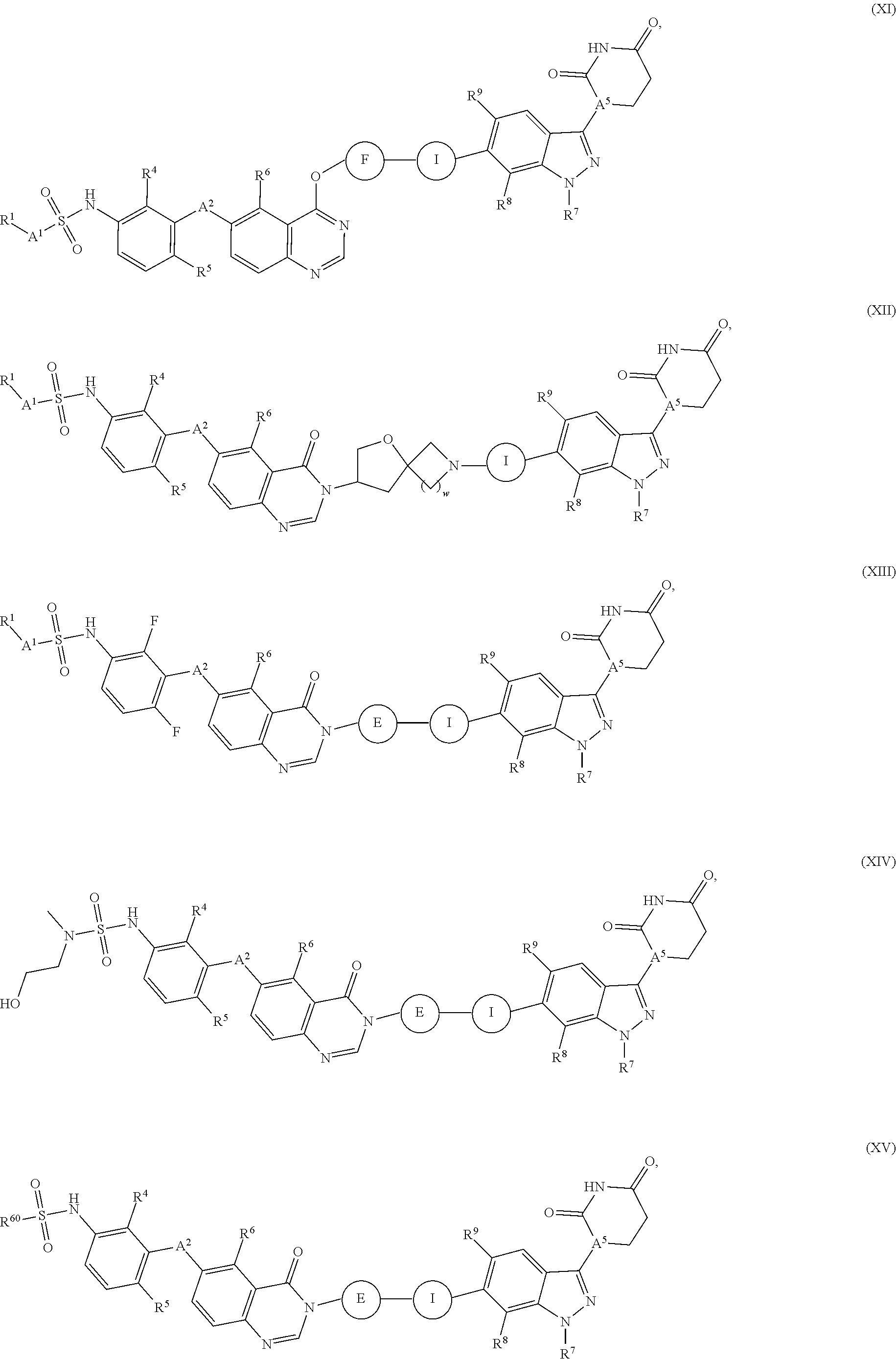

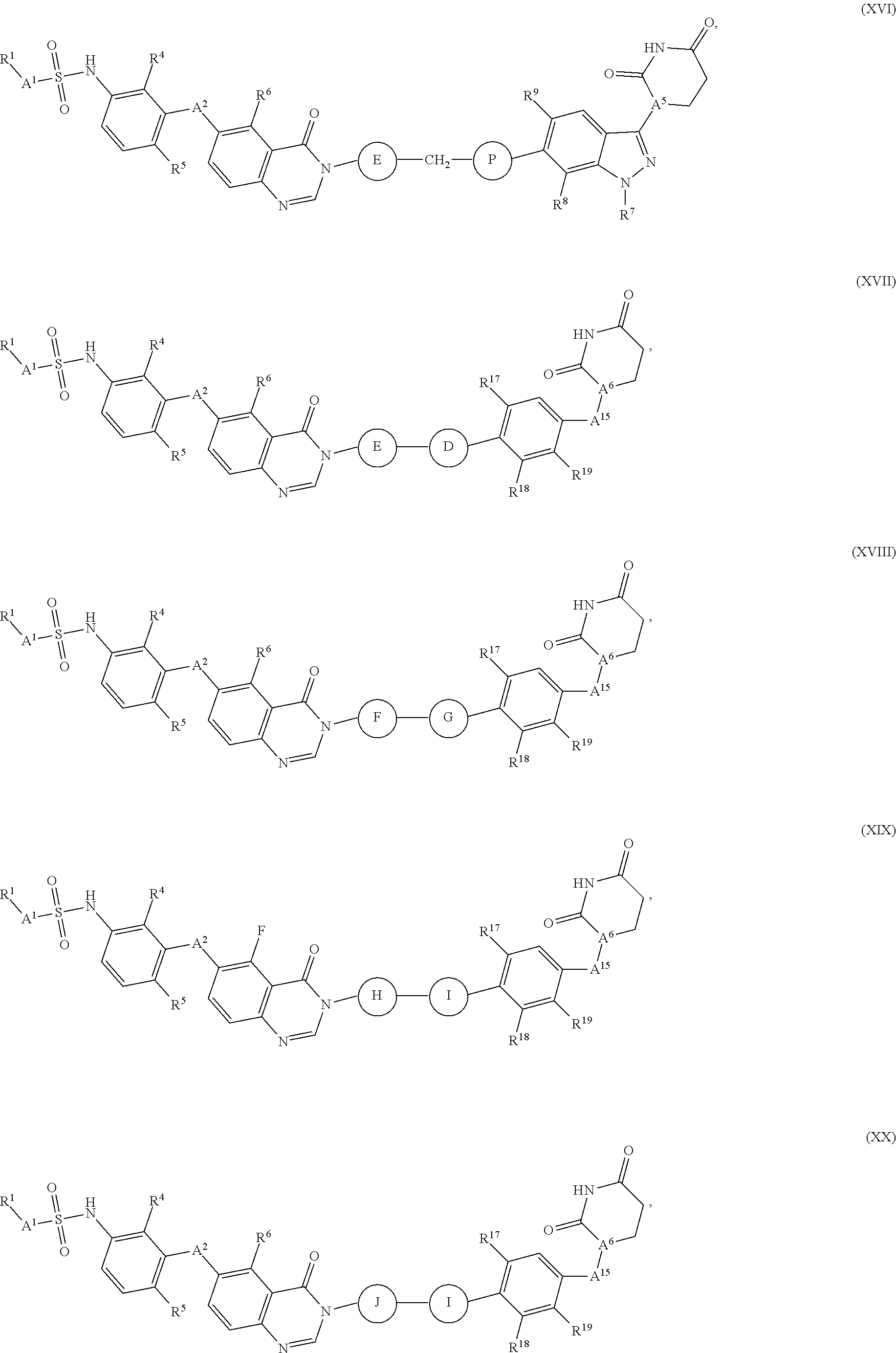

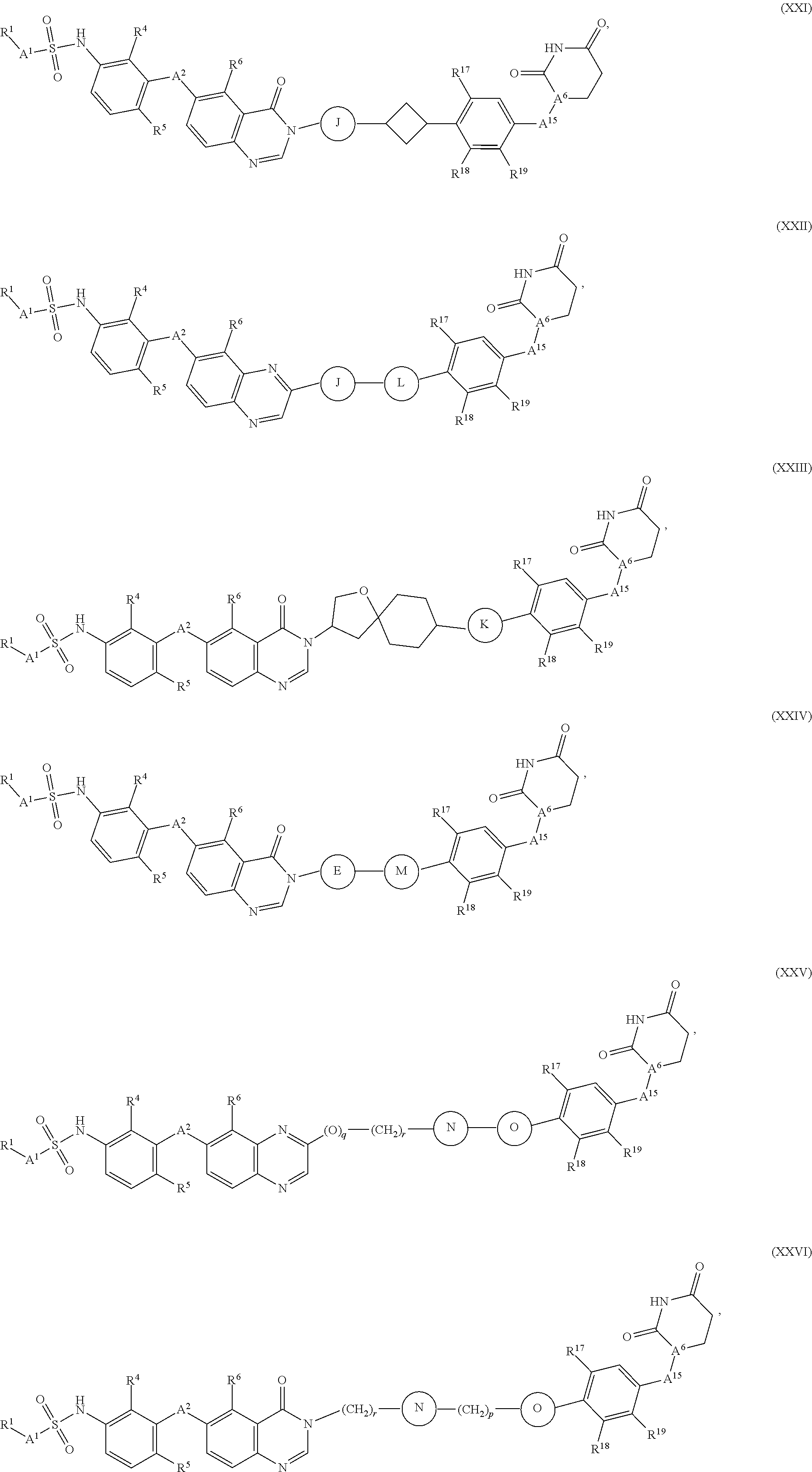

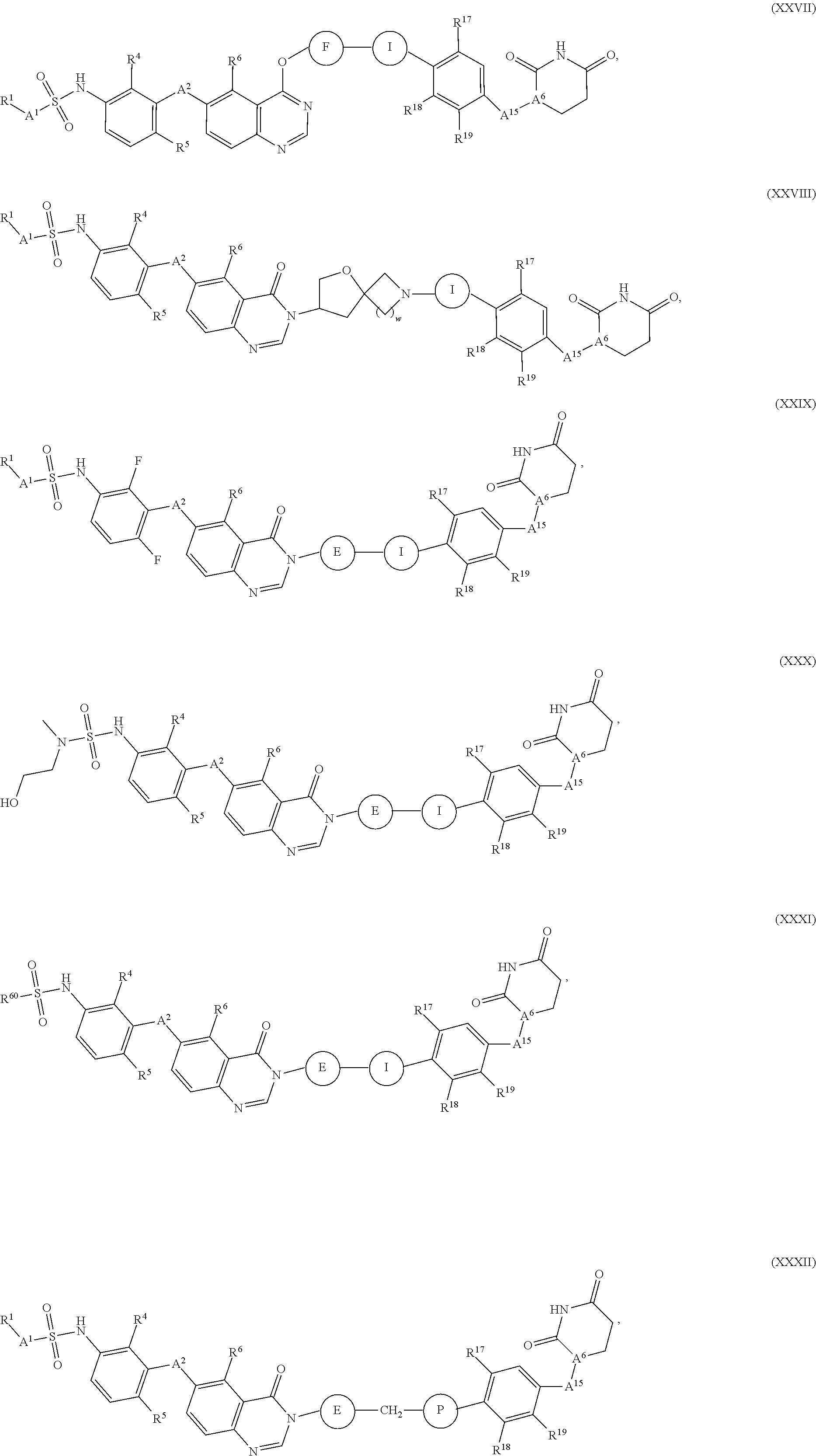

wherein the substituents and variables are as described herein, or a pharmaceutically acceptable salt thereof.

It has been surprisingly discovered that selected compounds of the present invention provide a fast and durable degradation of a mutant BRAF protein. For example, when tested in the BRAF V600E degradation assay of Example 5, Compound 26 and Compound 141 both degraded more than 50% of the BRAF V600E protein at a nanomolar concentration in just two hours.

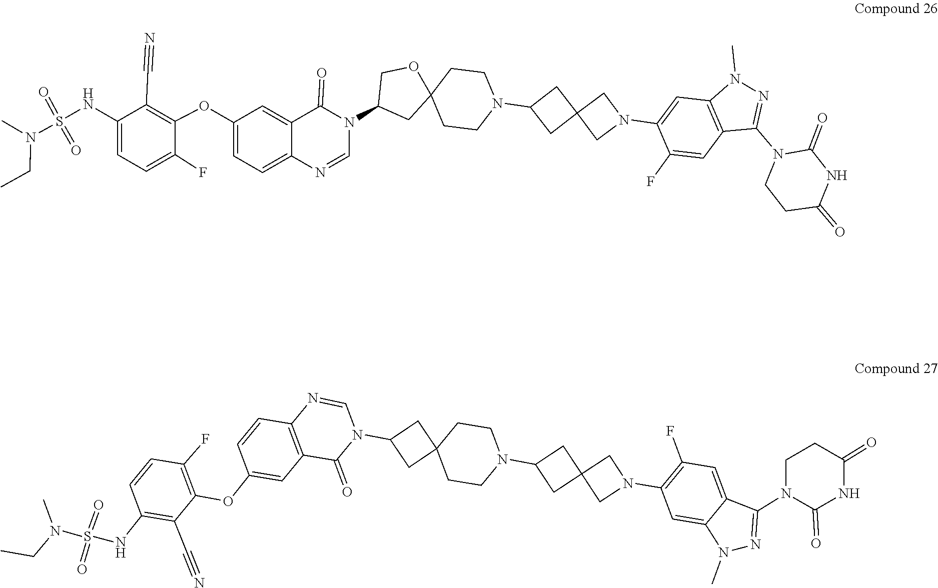 [SMILES: CCN(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N ; CCN(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1C#N]

This robust degradation activity is surprising in view of the minimal inhibition-based activity of selected compounds. Compound 26 and Compound 141 are potent cytotoxic agents in the A375 cell bioavailability assay of Example 15 and both compounds had a 30+% effect on cancer cell viability at a concentration of 100 nM. However, the degradation deficient versions of Compound 26 and Compound 141 (N-methyl analogs) had minimal effect on cell viability at a concentration of 100 nM (see FIG. 83 and Example 15). This experiment shows that selected compounds of the present invention exert their therapeutic effect by degrading a mutant BRAF instead of by both degrading and inhibiting the mutant BRAF.

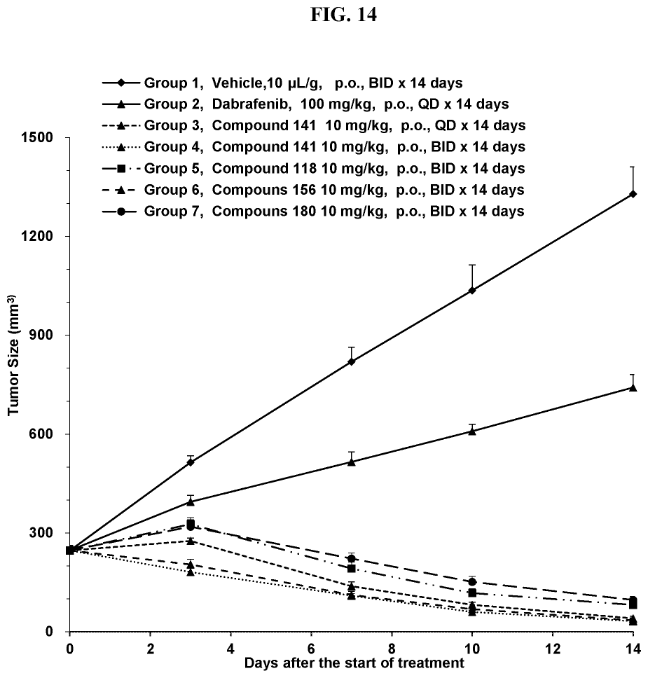

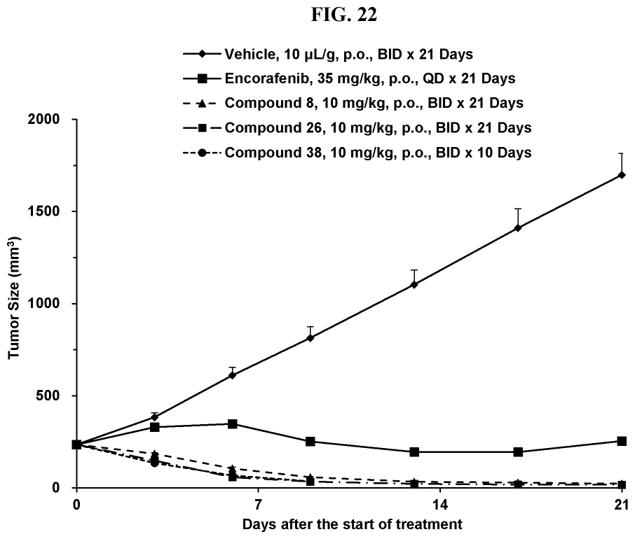

By degrading mutant BRAF, compounds of the present invention may be used in the treatment of a mutant BRAF mediated cancer. For example, in A375 human melanoma xenografts, oral delivery of Compound 8, Compound 118, Compound 141, Compound 156, or Compound 180 results in a more robust antitumor response than FDA approved BRAF inhibitors such as dabrafenib or encorafenib (see FIGS. 14 and 22). These compounds cause profound tumor regressions in the xenograft model when dosed at 10 mg/kg BID.

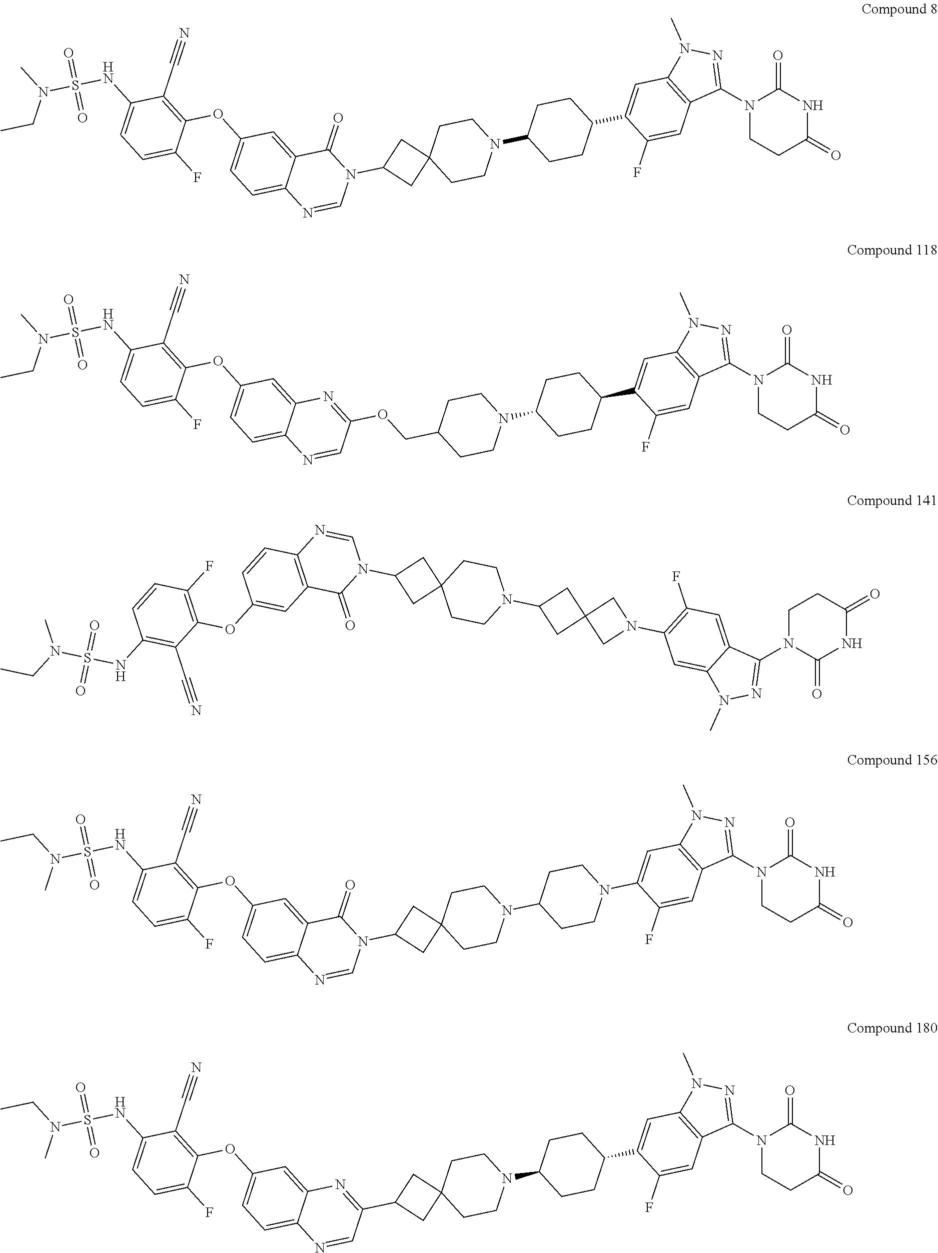

It has also been surprisingly discovered that a compound of the present invention has significant blood brain barrier penetration and can be used to treat a cancer that has metastasized to the brain. For example, CNS tumor-bearing mice that were administered Compound 26 or Compound 141 as described in Example 12 lived significantly longer than mice which were administered encorafenib. Samples taken from the CNS tumor-bearing mice show that BRAF V600E levels in the brain tumor are much lower after administration of Compound 26 or Compound 141 than after administration of encorafenib (see FIG. 51).

In certain aspects a method of treating a mutant BRAF mediated cancer that has metastasized to the brain or central nervous system (CNS) is provided comprising administering an effective amount of a compound of the present invention or a pharmaceutically acceptable salt thereof to a patient in need thereof. In certain embodiments the cancer that has metastasized to the brain or central nervous system is colorectal cancer, melanoma, or non-small cell lung cancer.

### Terminology

Compounds are described using standard nomenclature. Unless defined otherwise, all technical and scientific terms used herein have the same meaning as is commonly understood by one of ordinary skill in the art to which this invention belongs.

The compounds of the present invention may be in the form of a racemate, enantiomer, mixture of enantiomers, diastereomer, mixture of diastereomers, tautomer, N-oxide, isomer, such as rotamer, as if each is specifically described unless specifically excluded by context.

The terms “a” and “an” do not denote a limitation of quantity, but rather denote the presence of at least one of the referenced item(s). The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The present invention includes a compound of the present invention with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C, ¹⁴C, ¹⁵N, ¹⁷O, ¹⁸O ¹⁸F ³¹p, ³²P, ³⁵S, ³⁶Cl, and ¹²⁵I respectively. In certain embodiments, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In certain embodiments, deuterium is 90, 95 or 99% enriched at a desired location.

In certain embodiments, the substitution of a hydrogen atom for a deuterium atom can be provided in any compound of the present invention. In certain embodiments, the substitution of a hydrogen atom for a deuterium atom occurs within one or more groups selected from any of R's or variables described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle, an unsubstituted carbon may be deuterated.

In certain embodiments, a compound of the present invention is isotopically labeled. In certain embodiments, at least one R group independently selected from R¹, R², R^{2′}, R³, R⁴, R⁵, R⁶, R⁷, R⁸, R⁹, R¹⁷, R¹⁸, R¹⁹, and R⁶⁰ is isotopically labeled with 1, 2, or more isotopes as allowed by valence. In certain embodiments, the isotopic label is deuterium. In certain embodiments, at least one deuterium is placed on an atom that has a bond which is broken during metabolism of the compound in vivo, or is one, two or three atoms remote form the metabolized bond (e.g., which may be referred to as an a, p or y, or primary, secondary or tertiary isotope effect). In other embodiments, the isotopic label is ¹³C. In other embodiments, the isotopic label is ¹⁸F.

In certain non-limiting embodiments, the invention includes a solvated form of a compound described herein. The term “solvate” refers to a molecular complex of a compound of the present invention (including a salt thereof) with one or more solvent molecules. Non-limiting examples of solvents are water, ethanol, isopropanol, dimethyl sulfoxide, acetone, and other common organic solvents. The term “hydrate” refers to a molecular complex comprising a compound of the invention and water. Pharmaceutically acceptable solvates in accordance with the invention include those wherein the solvent may be isotopically substituted, e.g., D₂O, acetone-d₆, DMSO-d₆ (dimethyl sulfoxide). A solvate can be in a liquid or solid form.

A dash (“—”) that is not between two letters or symbols is used to indicate a point of attachment for a substituent. For example, —(C═O)NH₂ is attached through carbon of the carbonyl (C═O) group.

“Alkyl” is a branched or straight chain saturated aliphatic hydrocarbon group. Unless denoted otherwise, “alkyl” is typically a C₁-C₈ alkyl. In certain non-limiting embodiments, the alkyl group contains from 1 to 12 carbon atoms, more generally from 1 to 6 carbon atoms or from 1 to 4 carbon atoms. In certain non-limiting embodiments, the alkyl contains from 1 to 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species and therefore each subset is considered separately disclosed. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentyl, 3-methylpentyl, 2,2-dimethylbutyl, and 2,3-dimethylbutyl. The term “alkyl” also encompasses cycloalkyl or carbocyclic groups. For example, when a term is used that includes “alk” then “cycloalkyl” or “carbocyclic” can be considered part of the definition, unless unambiguously excluded by the context. For example, and without limitation, the terms alkyl, alkoxy, haloalkyl, etc., can all be considered to include the cyclic forms of alkyl, unless unambiguously excluded by context.

Non-limiting examples of “cycloalkyl” include dihydro-indene and tetrahydronaphthalene wherein the point of attachment for each group is on the cycloalkyl ring.

For example:

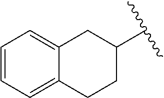 [SMILES: *C1CCc2ccccc2C1]

is an “cycloalkyl” group.

However,

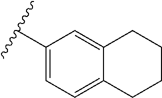 [SMILES: *c1ccc2c(c1)CCCC2]

is an “aryl” group.

The term “alkoxy” denotes a group of the formula —O-alkyl. Examples of alkoxy groups include methoxy, ethoxy, n-propoxy, isopropoxy, n-butoxy, isobutoxy, and tert-butoxy.

The term “cycloalkoxy” denotes a group of the formula —O-cycloalkyl. Examples of cycloalkoxy group include cyclopropoxy, cyclobutoxy, cyclopentyloxy, cyclohexyloxy, cycloheptyloxy, and cyclooctyloxy.

“Alkylene” is a bivalent saturated hydrocarbon. Alkylenes, for example, can be a 1, 2, 3, 4, 5, 6, 7 to 8 carbon moiety, 1 to 6-carbon moiety, or an indicated number of carbon atoms, for example C₁-C₂alkylene, C₁-C₃alkylene, C₁-C₄alkylene, C₁-C₅alkylene, or C₁-C₆alkylene.

The term “cyano” denotes a —C≡N group.

The term “hydroxy” denotes a —OH group.

“Halo” and “Halogen” refers independently to fluorine (F), chlorine (C₁), bromine (Br) or iodine (I). In typical embodiments halogen refers to fluorine.

“Haloalkyl” is a branched or straight-chain alkyl group substituted with 1 or more halo atoms described above, up to the maximum allowable number of halogen atoms. Unless denoted otherwise, “haloalkyl” is typically a C₁-C₄ haloalkyl. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include, but are not limited to, trifluoromethyl and pentafluoroethyl.

“Haloalkoxy” indicates a haloalkyl group as described herein attached through an oxygen bridge (oxygen of an alcohol radical).

“Heterocycloalkyl” is an alkyl group as described herein substituted with a heterocyclo group as described herein.

“Arylalkyl” is an alkyl group as described herein substituted with an aryl group as described herein.

Non-limiting examples of “arylalkyl” include:

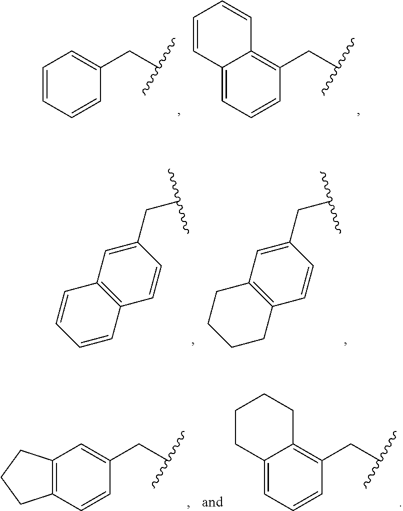

In certain embodiments, “arylalkyl” is

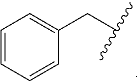 [SMILES: *Cc1ccccc1]

In certain embodiments, the “arylalkyl” refers to a 2-carbon alkyl group substituted with an aryl group.

Non-limiting examples of “arylalkyl” also include:

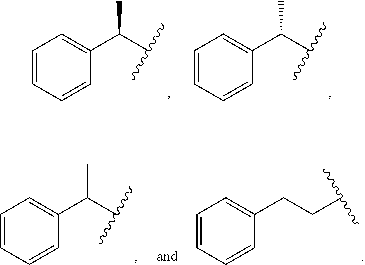

In certain embodiments, the “arylalkyl” refers to a 3-carbon alkyl group substituted with an aryl group.

“Heteroarylalkyl” is an alkyl group as described herein substituted with a heteroaryl group as described herein.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocycle groups wherein the radical or point of attachment is on the aryl ring, and in such instances, the number of carbon atoms continue to designate the number of carbon atoms in the aryl ring system. The one or more fused carbocyclyl or heterocycle groups can be 4 to 7 or 5 to 7-membered saturated or partially unsaturated carbocyclyl or heterocycle groups that optionally contain 1, 2, or 3 heteroatoms independently selected from nitrogen, oxygen, phosphorus, sulfur, silicon and boron, to form, for example, a 3,4-methylenedioxyphenyl group. In certain embodiments, aryl groups are pendant. An example of a pendant ring is a phenyl group substituted with a phenyl group.

In certain embodiments, “aryl” is a 6-carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example,

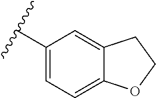 [SMILES: *c1ccc2c(c1)CCO2]

is an “aryl” group.

However,

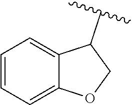 [SMILES: *C1COc2ccccc21]

is a “heterocycle” group.

In certain embodiments, “aryl” is a 6-carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example,

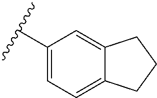 [SMILES: *c1ccc2c(c1)CCC2]

is an “aryl” group.

However,

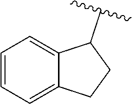 [SMILES: *C1CCc2ccccc21]

is a “cycloalkyl” group.

The term “heterocyclyl”, “heterocycle”, and “heterocyclo” includes saturated, and partially saturated heteroatom-containing ring radicals, where the heteroatoms may be selected from nitrogen, sulfur and oxygen. Heterocyclic rings comprise monocyclic 3, 4, 5, 6, 7, 8, 9, or 10 membered rings, as well as 5, 6, 7, 8, 9, 10, 11, 12, 13, 14, 15, or 16 membered bicyclic ring systems (which can include bridged fused and spiro-fused bicyclic ring systems). It does not include rings containing —O—O—, —O—S— or —S—S— portions. Examples of saturated heterocyclo groups include saturated 3, 4, 5, or 6-membered heteromonocyclic groups containing 1, 2, 3, or 4 nitrogen atoms [e.g., pyrrolidinyl, imidazolidinyl, piperidinyl, pyrrolinyl, piperazinyl]; saturated 3, 4, 5, or 6-membered heteromonocyclic group containing 1 or 2 oxygen atoms and 1, 2, or 3 nitrogen atoms [e.g., morpholinyl]; saturated 3, 4, 5, or 6-membered heteromonocyclic group containing 1 or 2 sulfur atoms and 1, 2, or 3 nitrogen atoms [e.g., thiazolidinyl]. Examples of partially saturated heterocycle radicals include, but are not limited to, dihydrothienyl, dihydropyranyl, dihydrofuryl, and dihydrothiazolyl. Examples of partially saturated and saturated heterocyclo groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl, isoquinolin-1(2H)-onyl, benzo[d]oxazol-2(3H)-onyl, 1,3-dihydro-2H-benzo[d]midazol-2-onyl, benzo[d]thiazole-2(3H)-onyl, 1,2-dihydro-3H-pyrazol-3-onyl, 2(1H)-pyridinonyl, 2-piperazinonyl, indolinyl, and dihydrothiazolyl.

The term “heterocyclyl”, “heterocycle”, and “heterocyclo” groups also include moieties where heterocycle radicals are fused/condensed with aryl or heteroaryl radicals: such as unsaturated condensed heterocycle group containing 1, 2, 3, 4, or 5 nitrogen atoms, for example, indoline, isoindoline, unsaturated condensed heterocycle group containing 1 or 2 oxygen atoms and 1, 2, or 3 nitrogen atoms, unsaturated condensed heterocycle group containing 1 or 2 sulfur atoms and 1, 2, or 3 nitrogen atoms, and saturated, partially unsaturated and unsaturated condensed heterocycle group containing 1 or 2 oxygen or sulfur atoms.

Additional non-limiting examples of “heterocycle” include indoline, tetrahydroquinoline, tetrahydroisoquinoline, and dihydrobenzofuran wherein the point of attachment for each group is on the heterocycle ring.

For example,

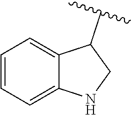 [SMILES: *C1CNc2ccccc21]

is a “heterocycle” group.

However,

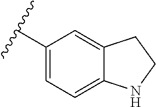 [SMILES: *c1ccc2c(c1)CCN2]

is an “aryl” group.

The term “heteroaryl” denotes a monocyclic or polycyclic (e.g., bicyclic or tricyclic) 4n+2 aromatic ring system (e.g., having 6, 10, or 14 π electrons shared in a cyclic array) and 1, 2, 3, 4, 5, or 6, heteroatoms independently selected from O, N, and S, wherein the ring nitrogen and sulfur atom(s) are optionally oxidized, and nitrogen atom(s) are optionally quarternized. Examples include, but are not limited to, unsaturated 5-to 6-membered heteromonocyclyl groups containing 1, 2, 3, or 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5-or 6-membered heteromonocyclic groups containing an oxygen atom, for example, pyranyl, 2-furyl, 3-furyl, etc.; unsaturated 5-or 6-membered heteromonocyclic groups containing a sulfur atom, for example, 2-thienyl, 3-thienyl, etc.; unsaturated 5-or 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 or 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. Additional examples include 8-, 9-, or 10-membered heteroaryl bicyclic groups such as indazolyl, indolyl, imidazo[1,5-a]pyridinyl, benzimidazolyl, 4(3H)-quinazolinonyl, quinolinyl, isoquinolinyl, isoindolyl, thienothienyl, indolizinyl, benzofuranyl, isobenzofuranyl, benzothienyl, isobenzothienyl, benzoxazolyl, benzothiazolyl, purinyl, coumarinyl, cinnolinyl, and triazolopyridinyl.

The term “bicycle” refers to a ring system wherein two rings are fused together and each ring is independently selected from carbocycle, heterocycle, aryl, and heteroaryl. Bicyclic ring systems also include spiro-fused bicyclic ring systems. Non-limiting examples of bicycle groups include:

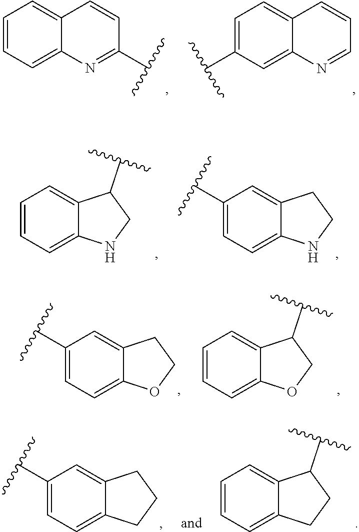

When the term “bicycle” is used in the context of a bivalent residue, the attachment points can be on separate rings or on the same ring. In certain embodiments, both attachment points are on the same ring. In certain embodiments, both attachment points are on different rings. Non-limiting examples of bivalent bicycle groups include:

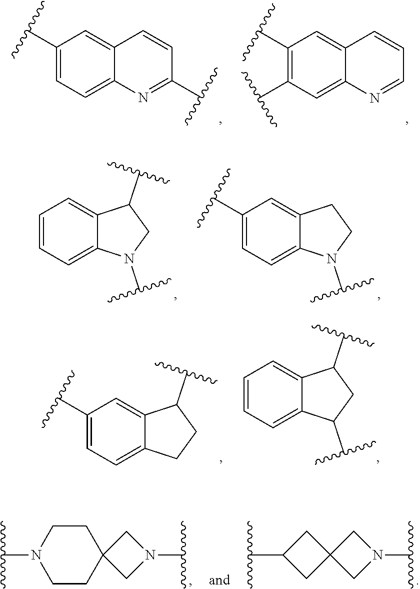

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

The term “sulfonyl”, alone or in combination with other groups, is the group —SO₂—.

The term “as defined herein” and “as described herein” when referring to a variable incorporates by reference the broad definition of the variable as well as particularly, more particularly and most particularly definitions, if any.

The term “inhibitor” denotes a compound which competes with, reduces or prevents the binding of a particular ligand to particular receptor, or which reduces or prevents the function of a particular protein.

If one of the starting materials or compounds the present invention contain one or more functional groups which are not stable or are reactive under the reaction conditions of one or more reaction steps, appropriate protecting groups (as described e.g., in “Protective Groups in Organic Chemistry” by T. W. Greene and P. G. M. Wuts, 3rd Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of the present invention can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

Whenever a chiral carbon is present in a chemical structure, it is intended that all stereoisomers associated with that chiral carbon are encompassed by the structure as pure stereoisomers as well as mixtures thereof.

A compound of the invention can exist as a tautomer, i.e., a structural isomer which interconverts with the compound of the present invention as drawn herein, in particular in solution. It is intended that the compound of the present invention encompasses all existing tautomeric forms thereof.

A compound of the invention can exist as a solvate. It is intended that the compound of the invention encompasses all existing solvates thereof.

The invention also provides pharmaceutical compositions, methods of using, and methods of preparing the aforementioned compounds.

The compounds of the invention may contain one or more asymmetric centers and can therefore occur as racemates, mixtures of enantiomers, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains greater than 90% of the desired isomer by weight, particularly greater than 95% of the desired isomer by weight, or more particularly greater than 99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

### Embodiments of Formula I and Formula XVII

In certain embodiments a compound of the invention is selected from the compound of Formula I and the compound of Formula XVII:

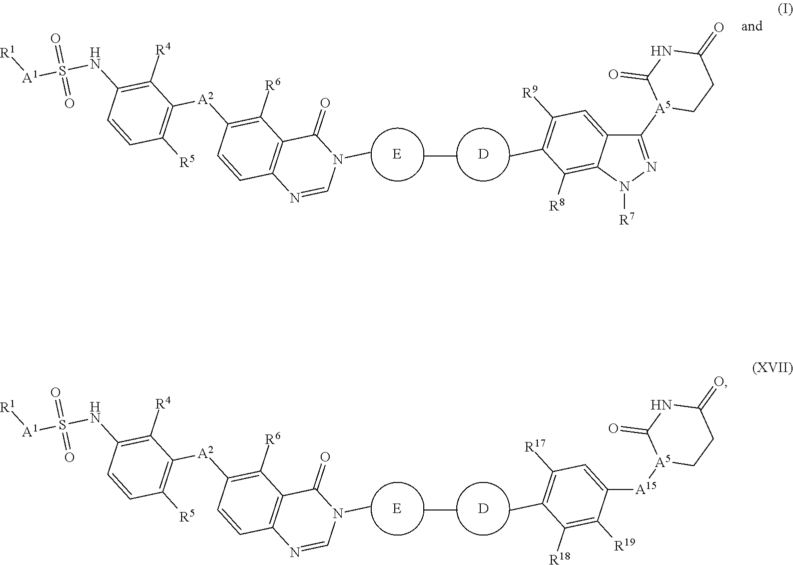

or a pharmaceutically acceptable salt thereof,
wherein
Cycle \textcircled{D} is a spiroheterocyclyl containing 1 or 2 nitrogen atoms and containing no other heteroatoms, bridged cycloalkyl, bridged heterocyclyl, orpiperidine-1,4-diyl, wherein
cycle \textcircled{D} is optionally substitutedwith one or two substituents independently selected from halogen, alkyl and alkoxy;
Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
A¹ is selected from —NR²— and —CUR^{2′}—;
R¹ is selected from hydrogen, alkyl and cycloalkyl;
R² is selected from hydrogen, alkyl, cycloalkyl and haloalkyl;
or R¹ and R² together with the nitrogen atom to which they are attached form heterocycloalkyl optionally substituted with one or two R³;
R^{2′} is selected from hydrogen, alkyl, cycloalkyl and haloalkyl;
or R¹ and R^{2′} together with the carbon atom to which they are attached form cycloalkyl optionally substituted with one or two R³;
each R³ is independently selected from hydrogen, halogen, alkyl, cycloalkyl and alkoxy;
R⁴ is selected from hydrogen, alkyl, cyano and halogen;
R⁵ is selected from hydrogen, alkyl, cyano and halogen;
A² is selected from —O—, —NH— and —(C═O)—;
R⁶ is selected from hydrogen, halogen, hydroxy, amino, dialkylamino, alkoxy, alkyl and alkoxyalkyl;
R⁷ is selected from hydrogen and alkyl;
R⁸ is selected from hydrogen, alkyl, cyano, halogen, and alkoxy;
R⁹ is selected from hydrogen, alkyl, cyano, halogen, and alkoxy;
A⁵ is —CH— or —N—;
R¹⁷ is selected from hydrogen, alkyl, cyano, hydroxy, cycloalkyl, halogen and alkoxy;
R¹⁸ is selected from hydrogen, alkyl, cyano, hydroxy, cycloalkyl, halogen and alkoxy;
R¹⁹ is selected from hydrogen, alkyl, cyano, hydroxy, cycloalkyl, halogen and alkoxy;
A¹⁵ is selected from a bond, —O— and —NH—, wherein A⁶ is CH when A¹⁵ is O or NH and wherein A⁶ is N or CH when A¹⁵ is bond; and
A⁶ is —CH— or —N—.

In certain embodiments the compound of Formula I or Formula XVII is selected from:

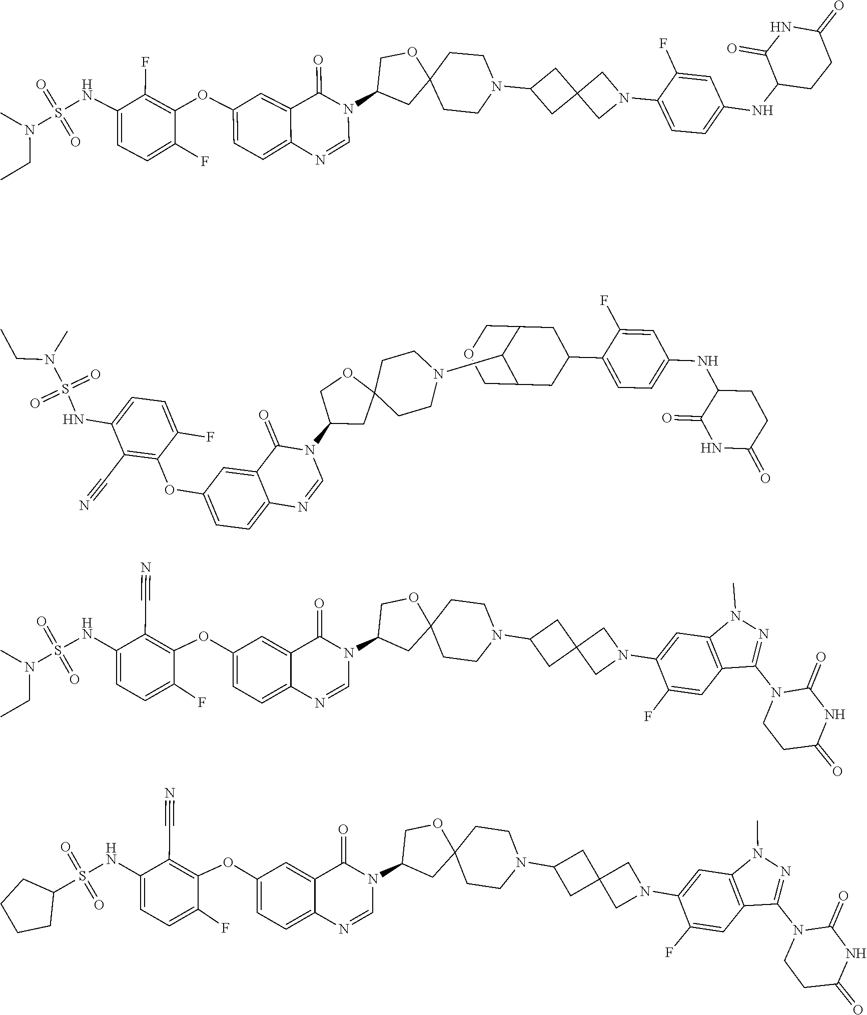 [SMILES: CCN(C)S(=O)(=O)Nc1ccc(F)c(Oc2ccc3ncn([C@H]4COC5(CCN(C6CC7(C6)CN(c6ccc(NC8CCC(=O)NC8=O)cc6F)C7)CC5)C4)c(=O)c3c2)c1F ; CCN(C)S(=O)(=O)Nc1ccc(F)c(Oc2ccc3ncn([C@H]4COC5(CCN(C6C7COCC6CC(c6ccc(NC8CCC(=O)NC8=O)cc6F)C7)CC5)C4)c(=O)c3c2)c1C#N ; CCN(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N ; Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CCCC9)c8C#N)cc7c5=O)CO6)C4)C3)cc21]

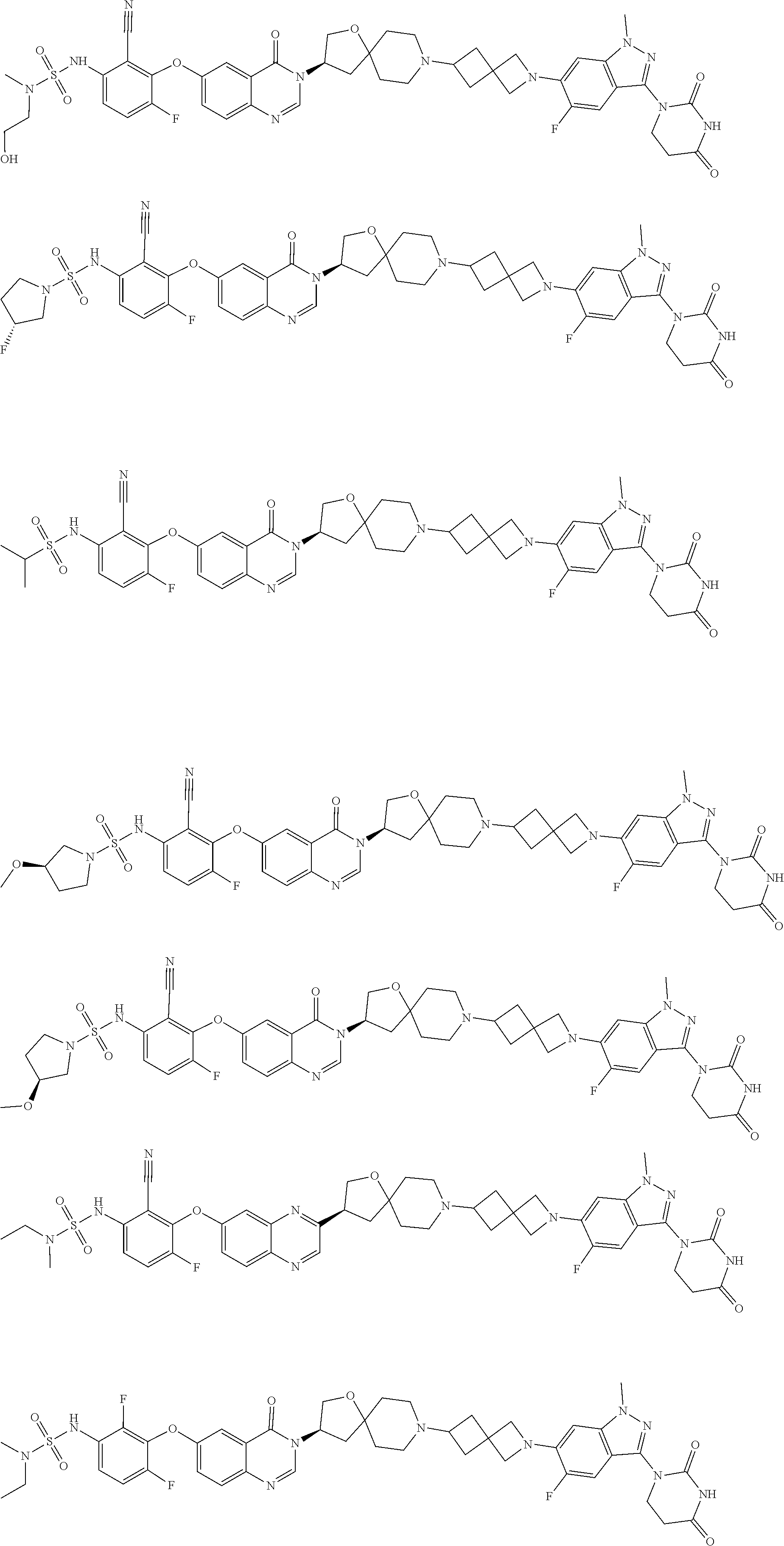

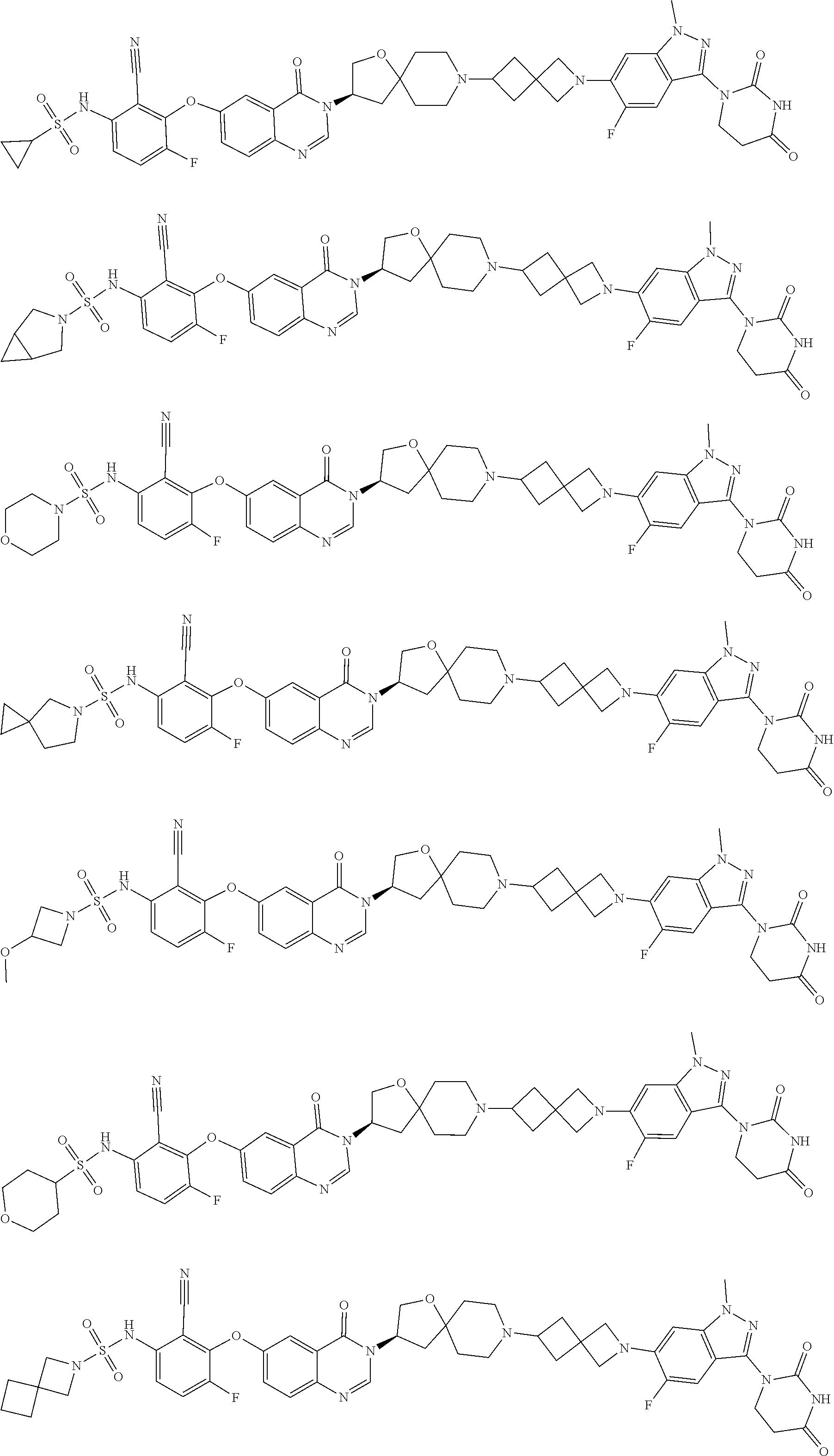

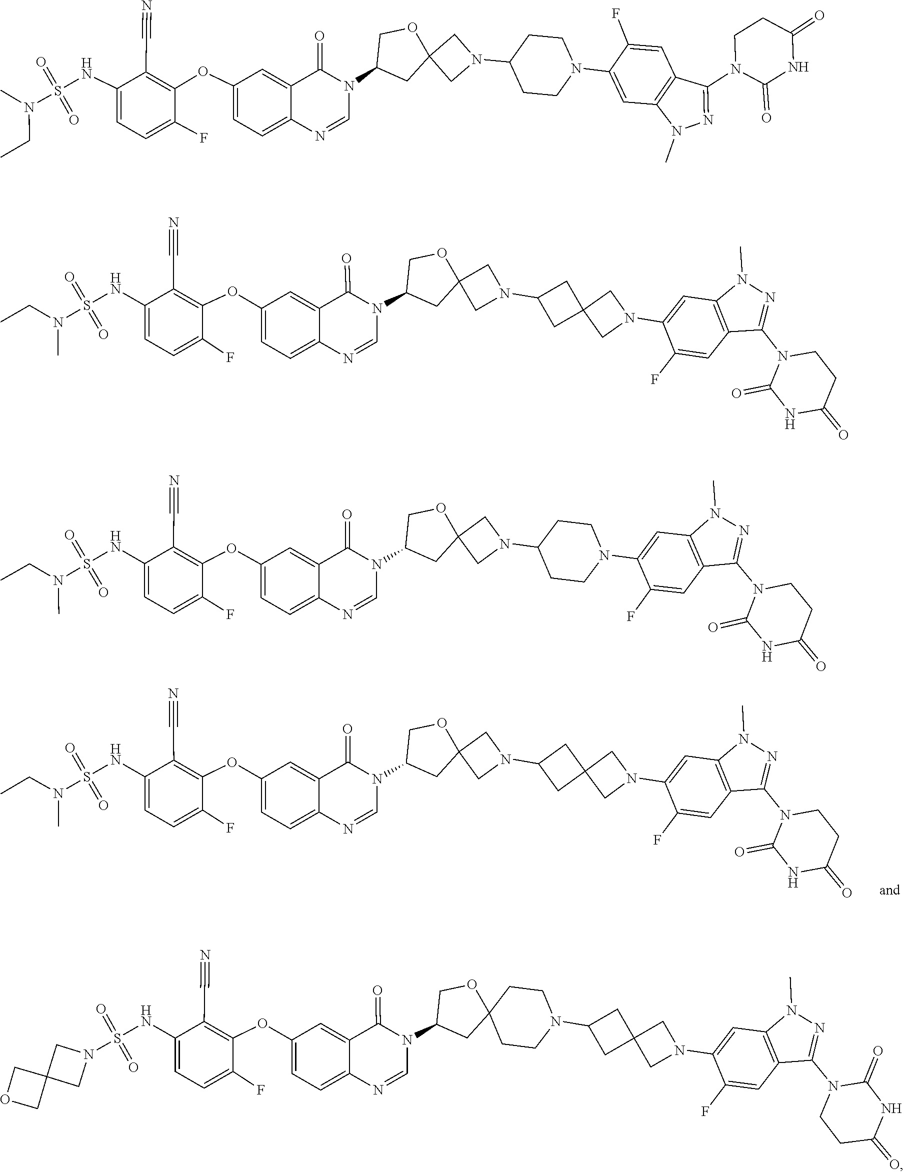

or pharmaceutically acceptable salt thereof.

The invention further provides the following.

A compound of Formula I or Formula XVII or a pharmaceutically acceptable salt thereof, for use as a therapeutically active substance in the treatment of a mutant BRAF mediated disorder.

A pharmaceutical composition comprising a compound of Formula I or Formula XVII or a pharmaceutically acceptable salt thereof, and a pharmaceutically acceptable carrier.

The use of a compound of Formula I or Formula XVII or a pharmaceutically acceptable salt thereof, for the therapeutic and/or prophylactic treatment of cancer.

A compound of Formula I or Formula XVII or a pharmaceutically acceptable salt thereof, for use in the treatment and/or prophylaxis of cancer.

The use of a compound of Formula I or Formula XVII or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula I or Formula XVII or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula II and Formula XVIII

In certain embodiments a compound of the invention is selected from the compound of Formula II and the compound of Formula XVIII:

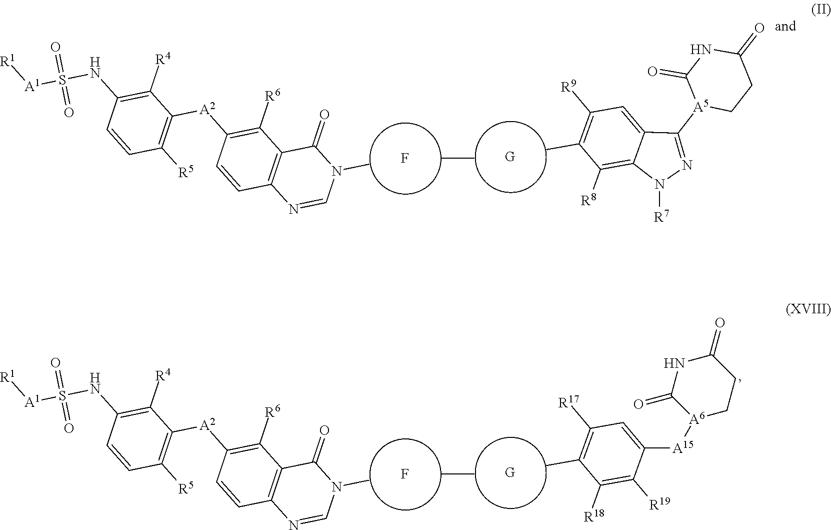

or a pharmaceutically acceptable salt thereof,
wherein:
Cycle \textcircled{F} is a bridged heterocyclyl, fused heterocyclyl, or spiroheterocyclyl containing 1 or 2 nitrogen atoms and containing no other heteroatoms, wherein cycle \textcircled{F} is optionally substituted with one or two substituents independently selected from halogen, alkyl and alkoxy;
Cycle \textcircled{G} is a cycloalkyl or heterocyclyl containing 1 or 2 nitrogen atoms and 0 or 1 oxygen atom and containing no other heteroatoms, wherein cycle \textcircled{G} is optionally substituted with one or two substituents independently selected from halogen, alkyl and alkoxy; and all other substituents are as defined herein.

In certain embodiments the compound of Formula II or Formula XVIII is selected from:

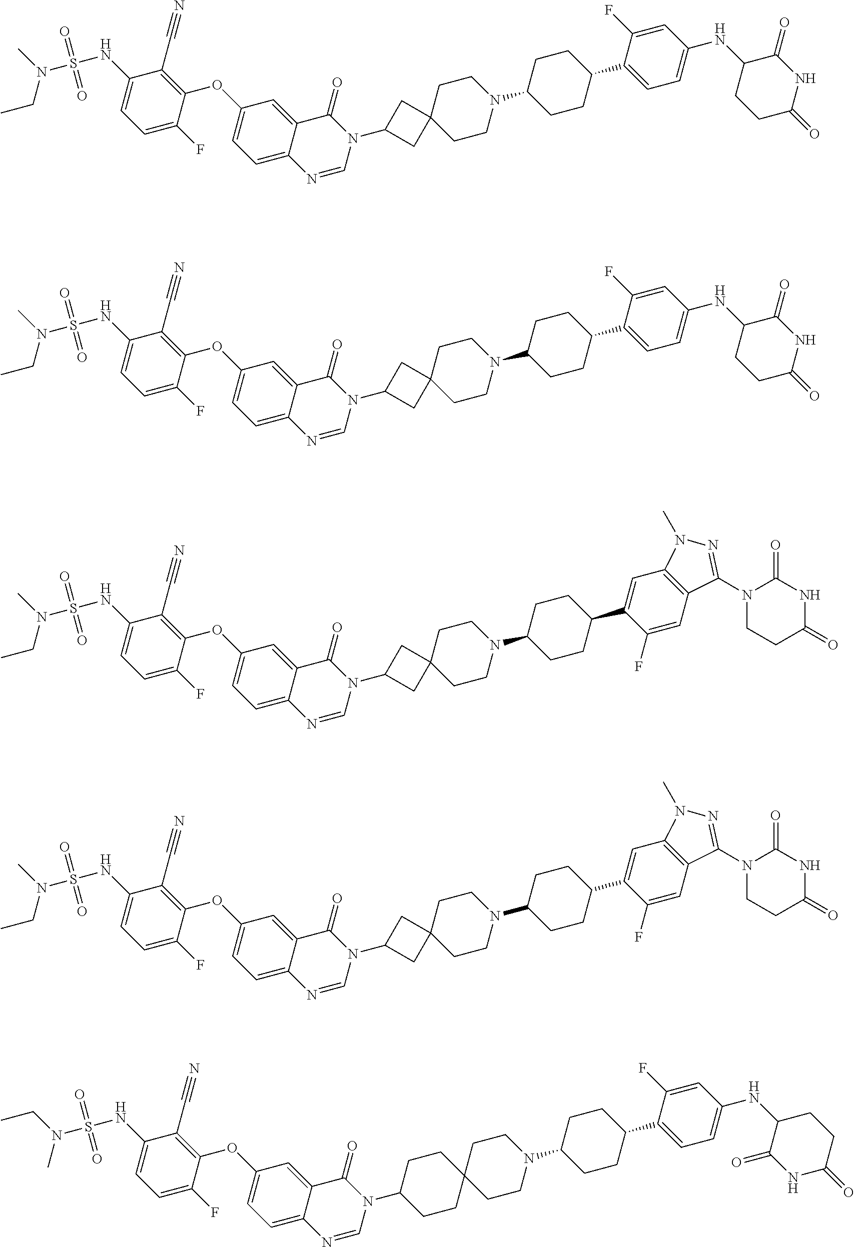

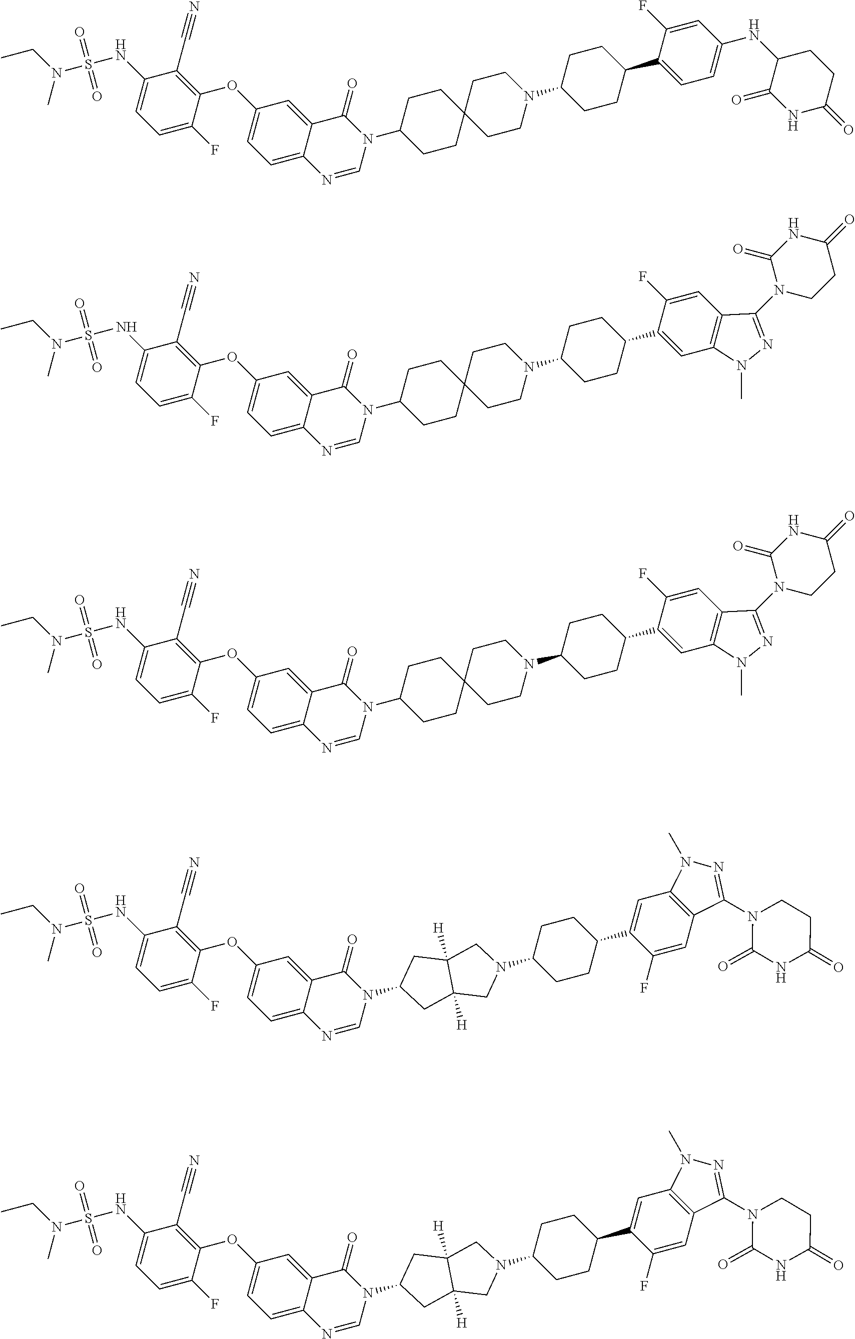

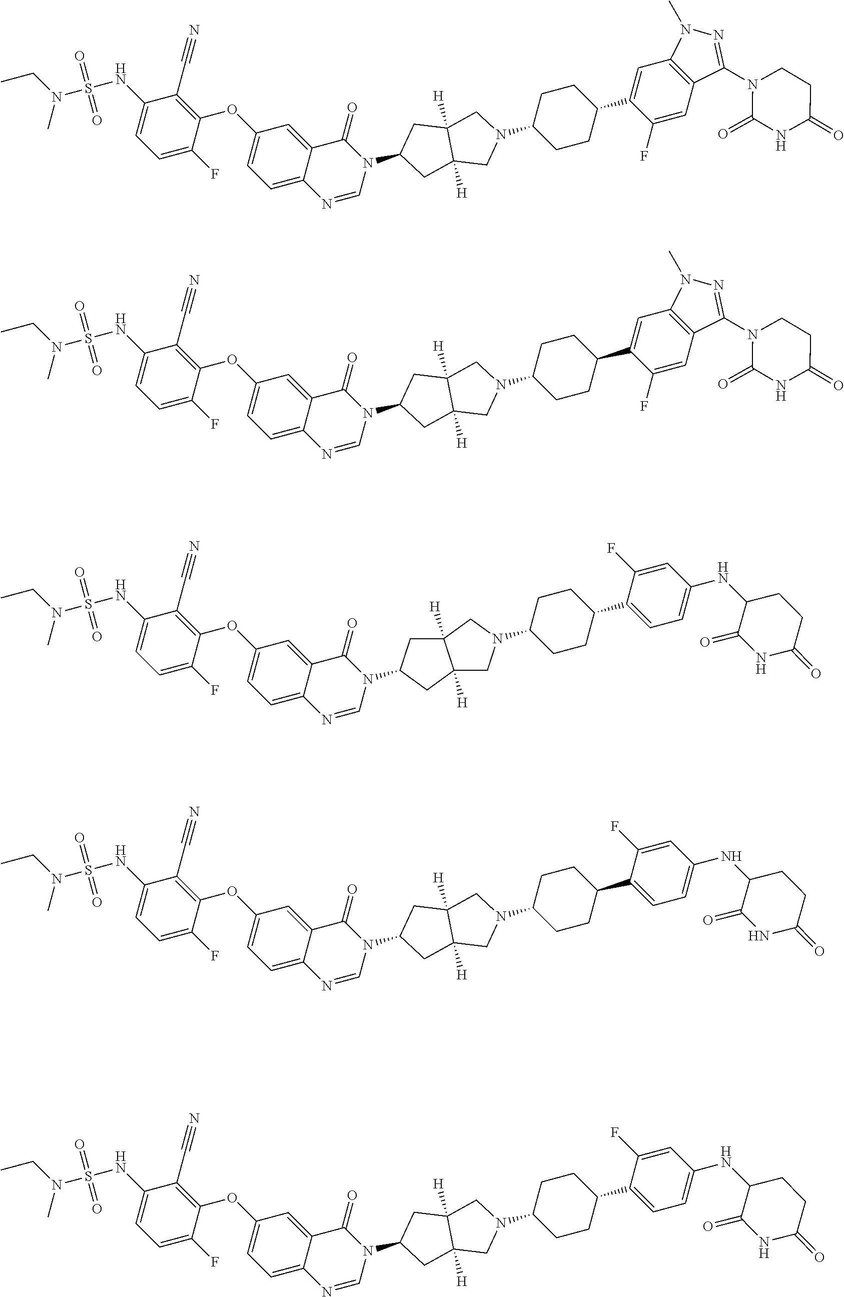

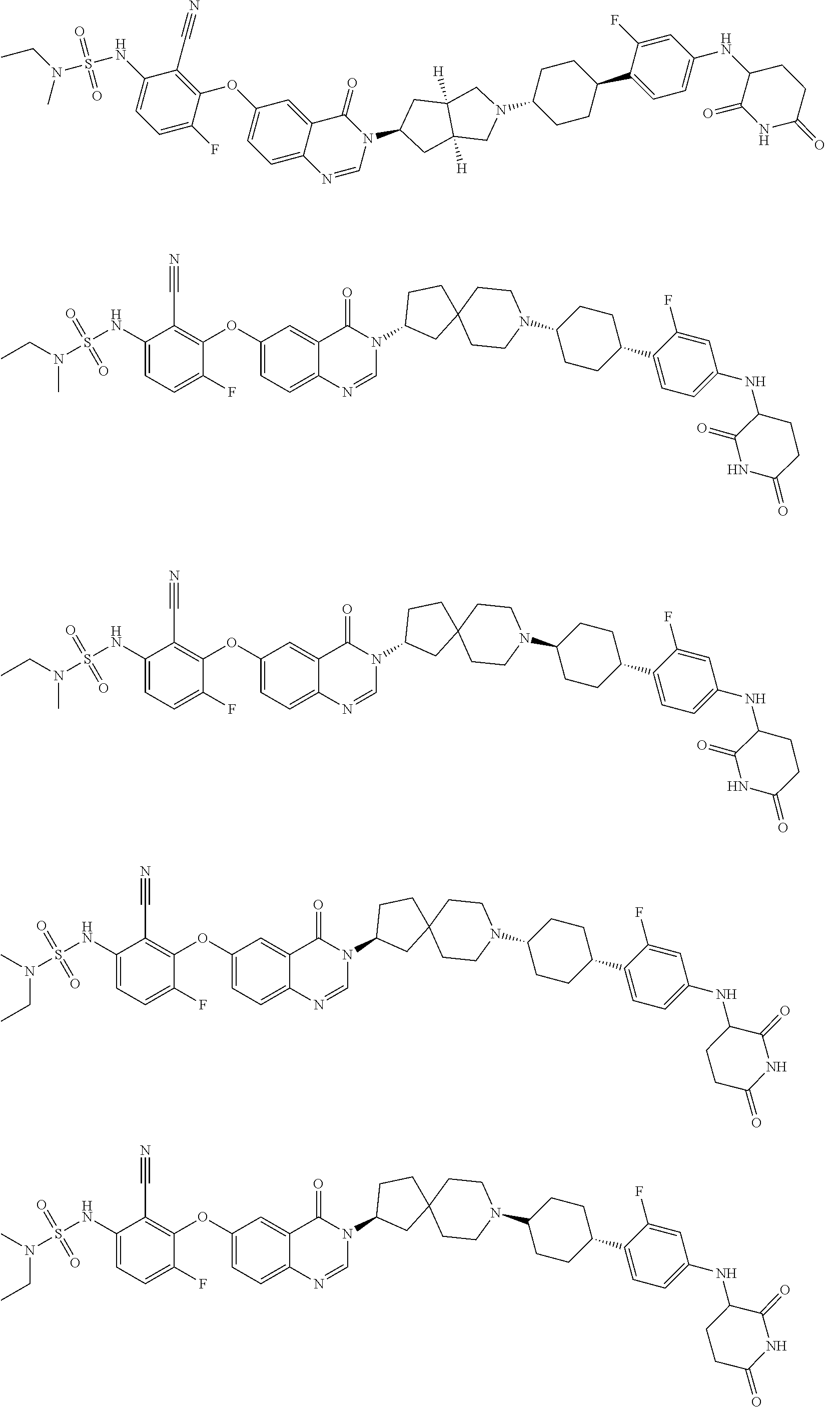

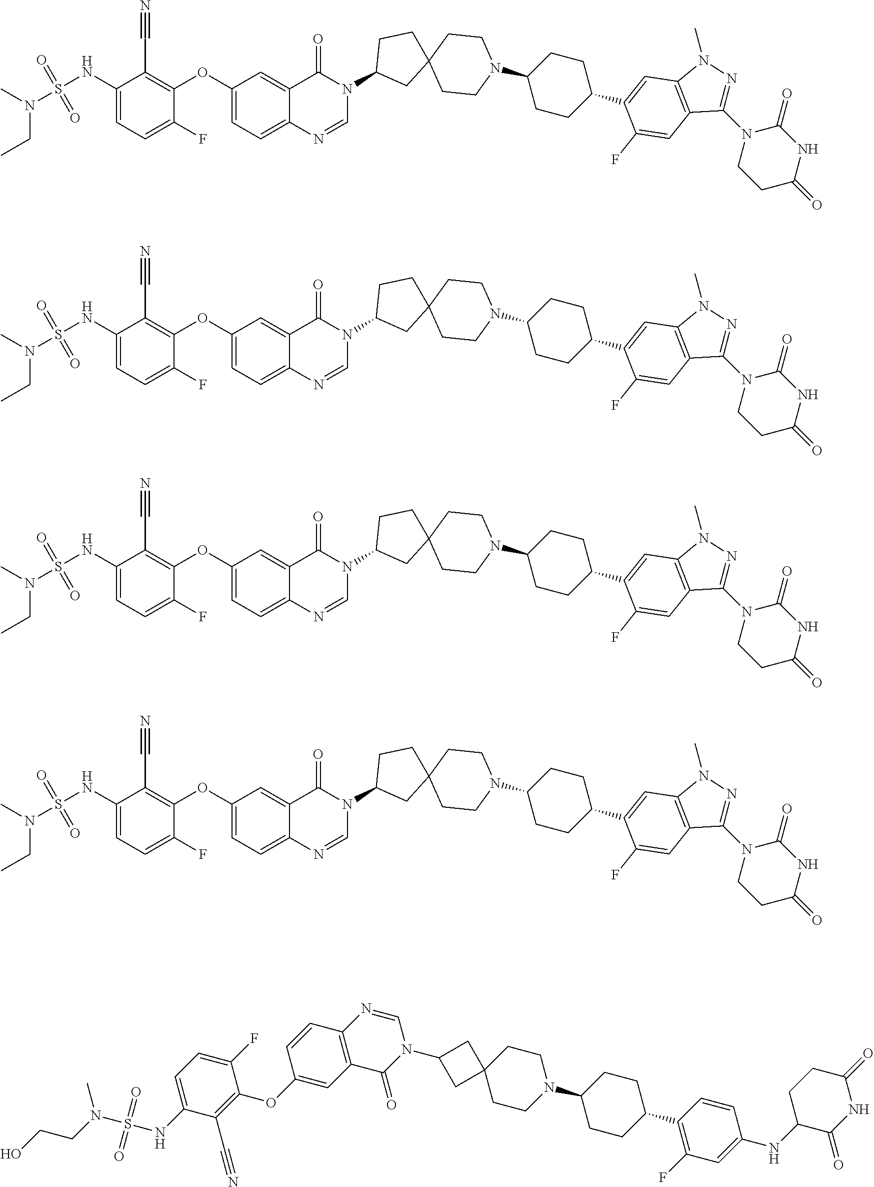

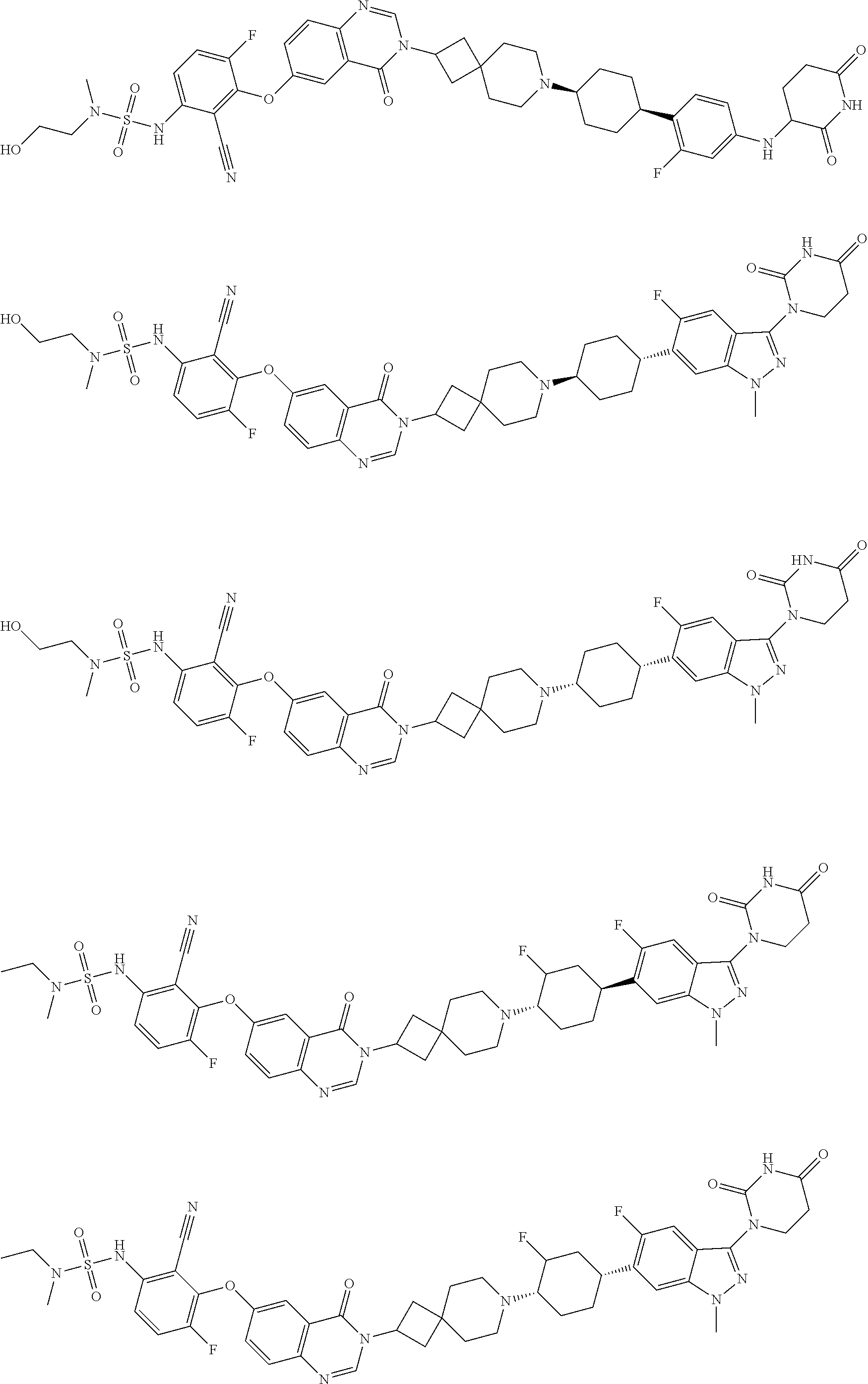

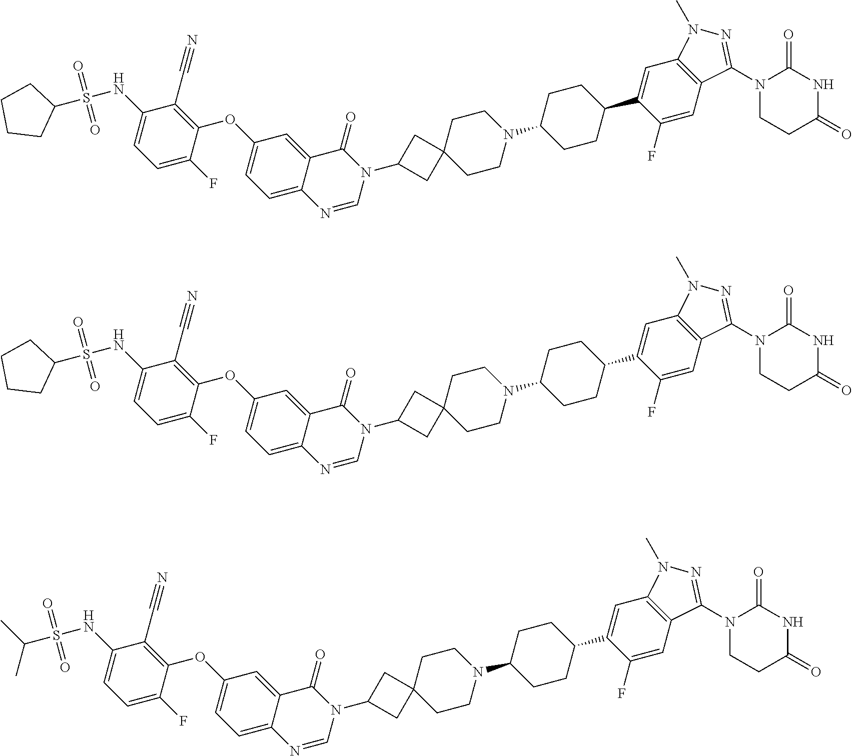 [SMILES: Cn1nc(N2CCC(=O)NC2=O)c2cc(F)c([C@H]3CC[C@H](N4CCC5(CC4)CC(n4cnc6ccc(Oc7c(F)ccc(NS(=O)(=O)C8CCCC8)c7C#N)cc6c4=O)C5)CC3)cc21 ; Cn1nc(N2CCC(=O)NC2=O)c2cc(F)c([C@H]3CC[C@@H](N4CCC5(CC4)CC(n4cnc6ccc(Oc7c(F)ccc(NS(=O)(=O)C8CCCC8)c7C#N)cc6c4=O)C5)CC3)cc21 ; CC(C)S(=O)(=O)Nc1ccc(F)c(Oc2ccc3ncn(C4CC5(CCN([C@H]6CC[C@H](c7cc8c(cc7F)c(N7CCC(=O)NC7=O)nn8C)CC6)CC5)C4)c(=O)c3c2)c1C#N]

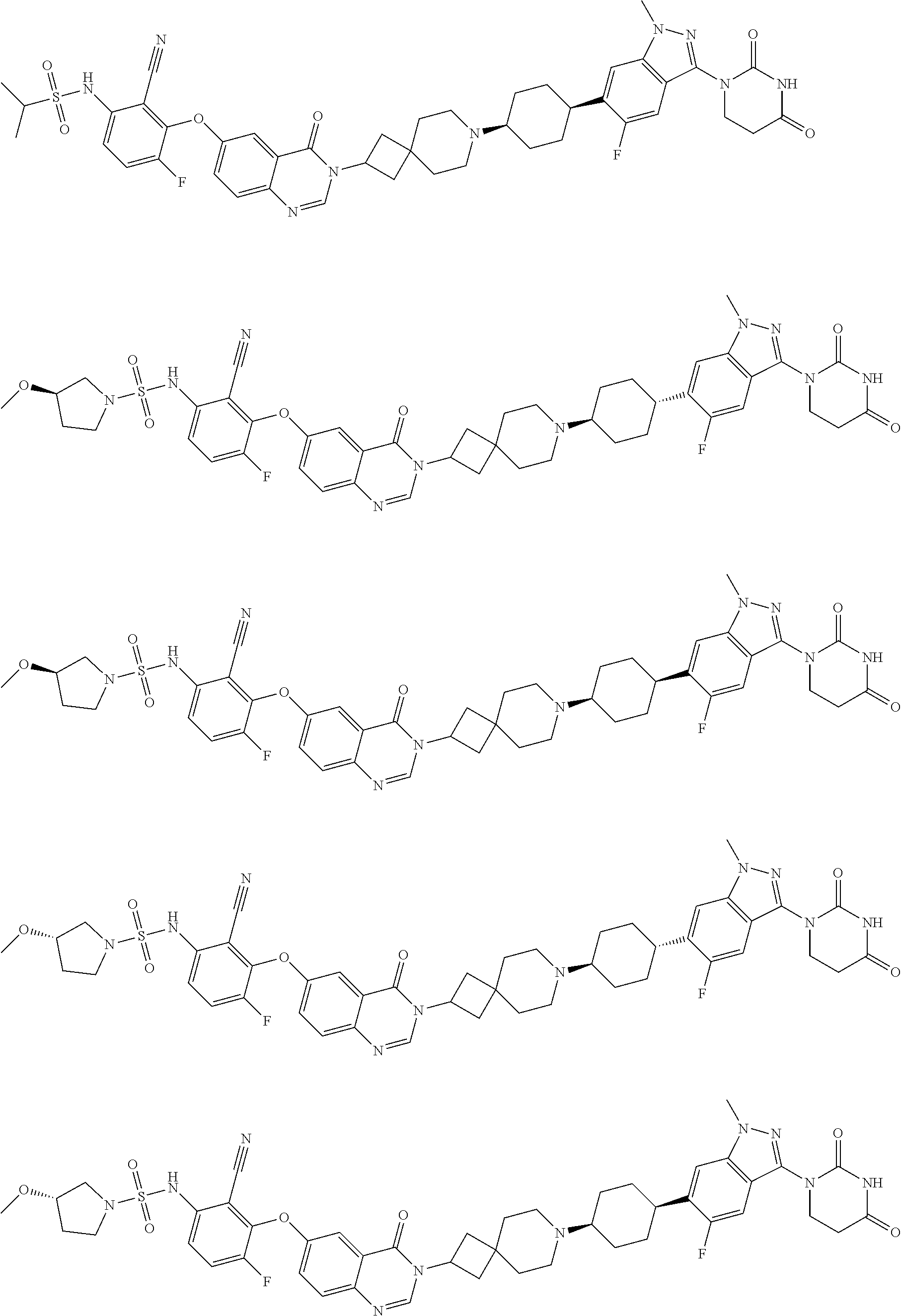

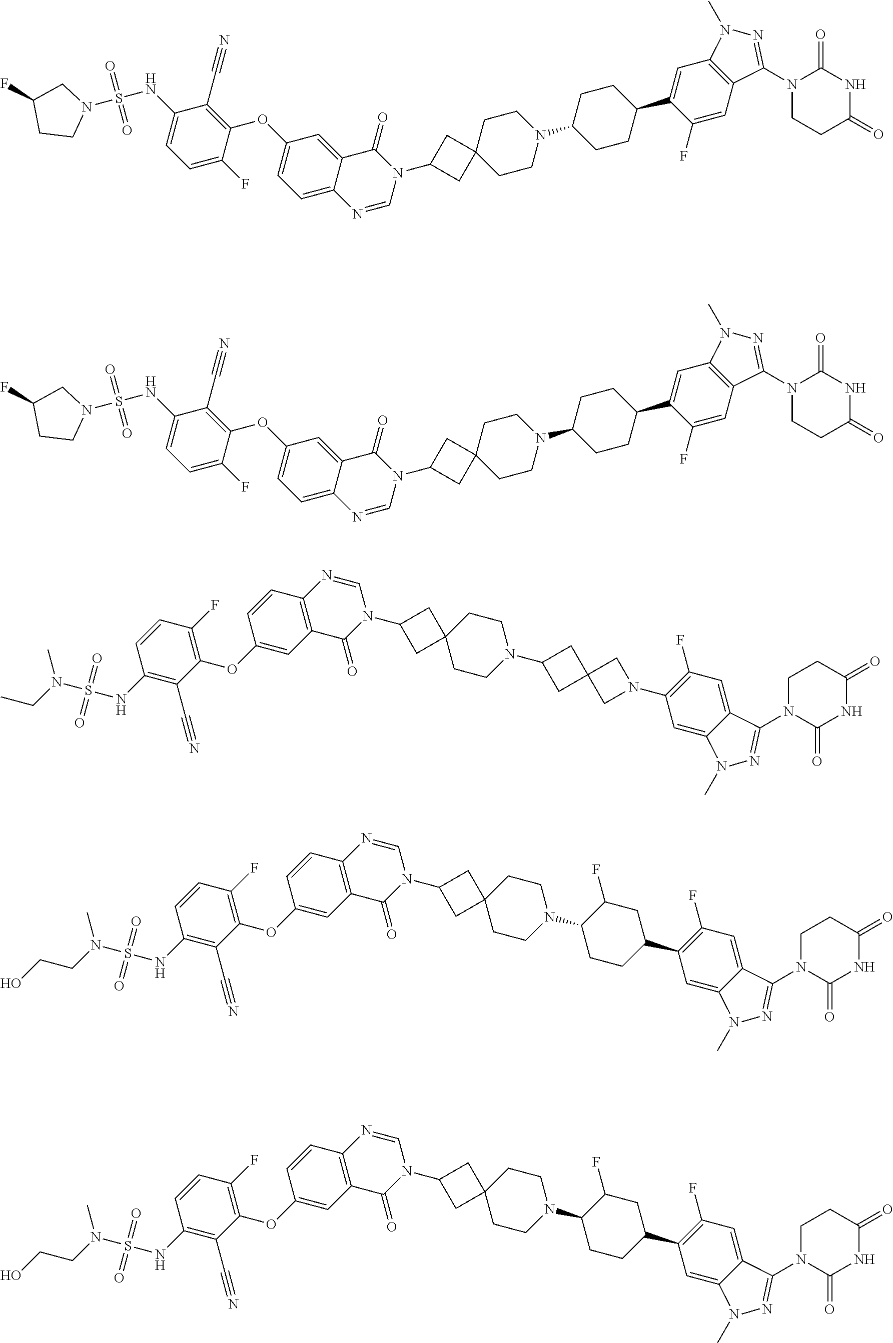

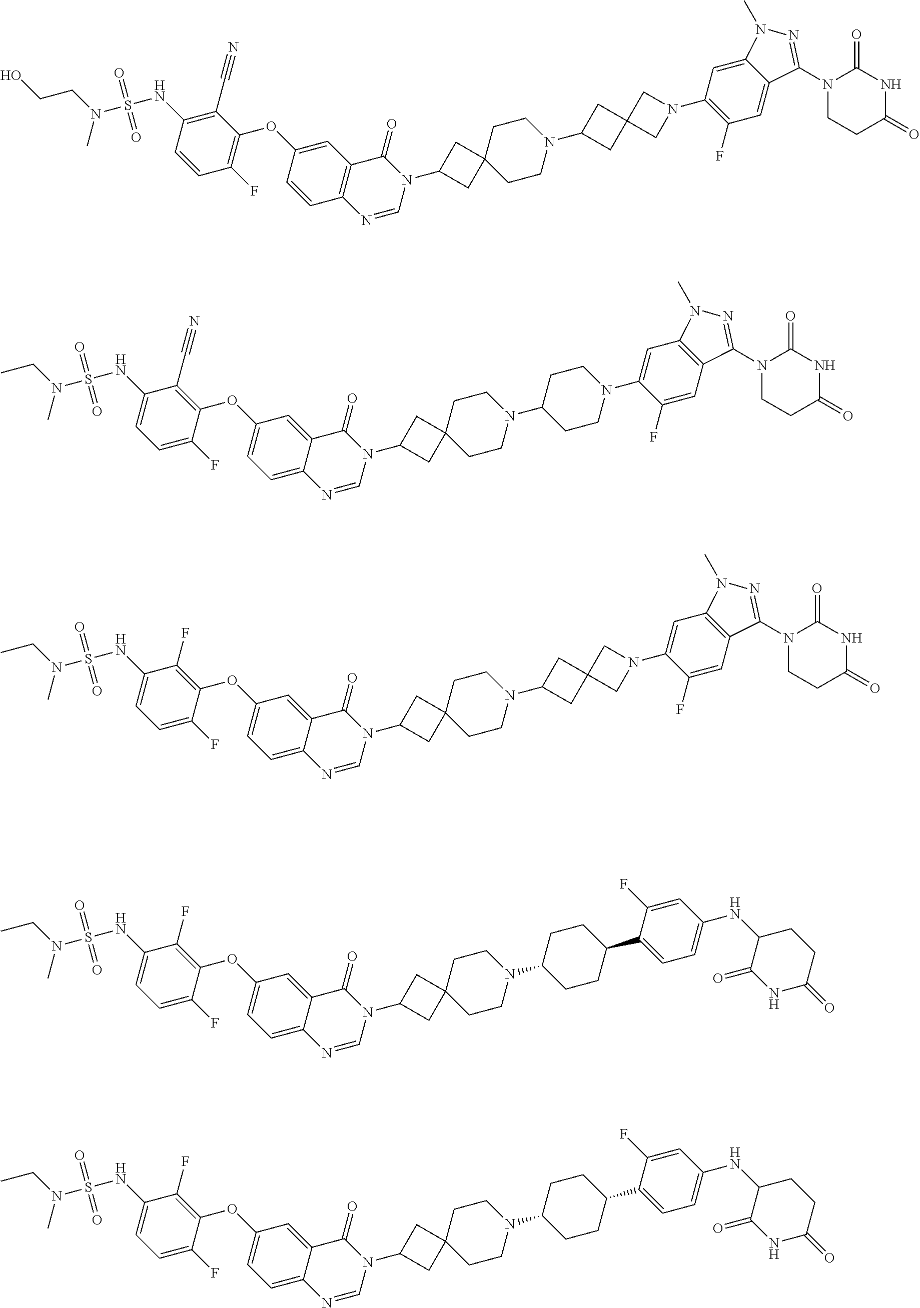

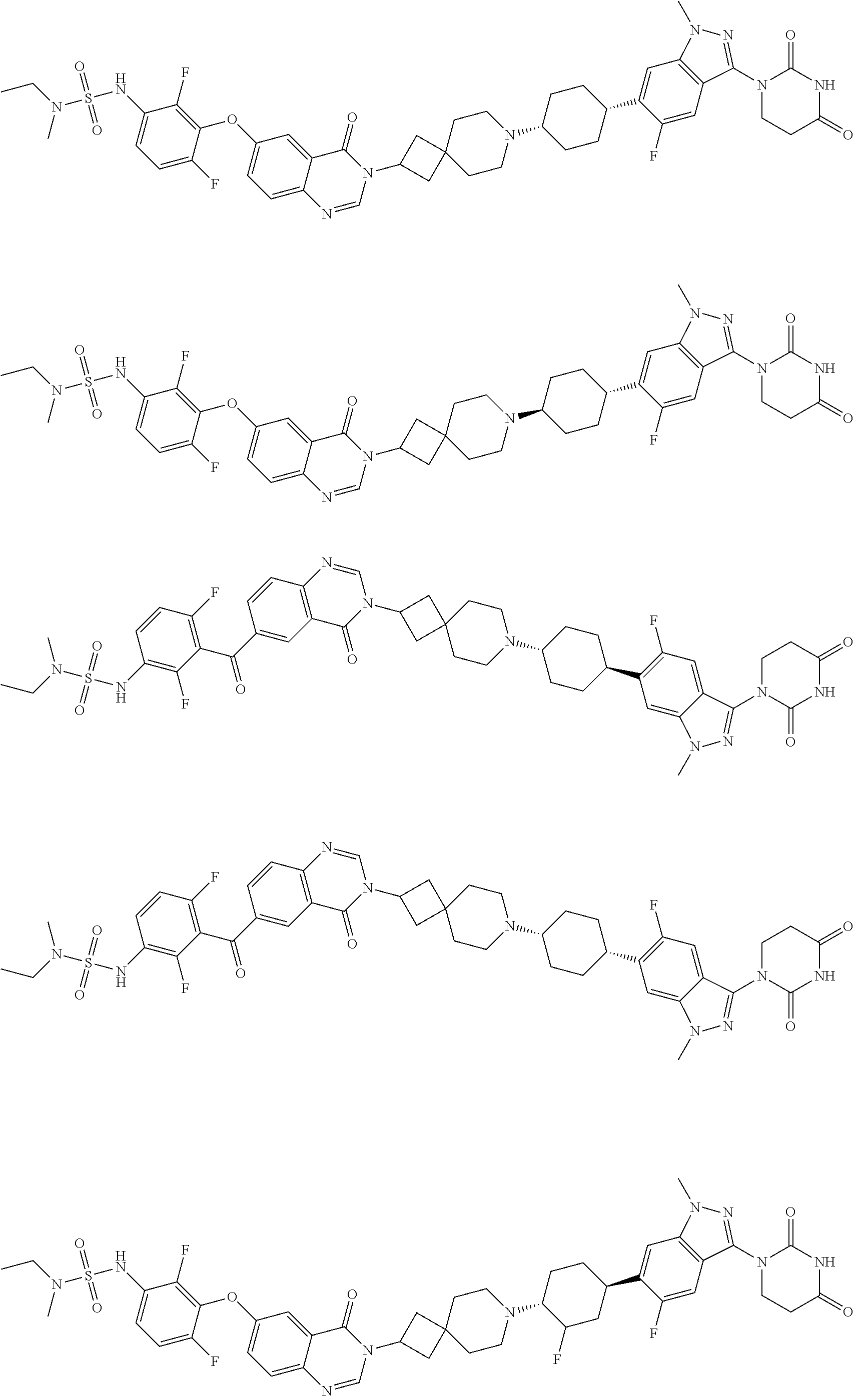

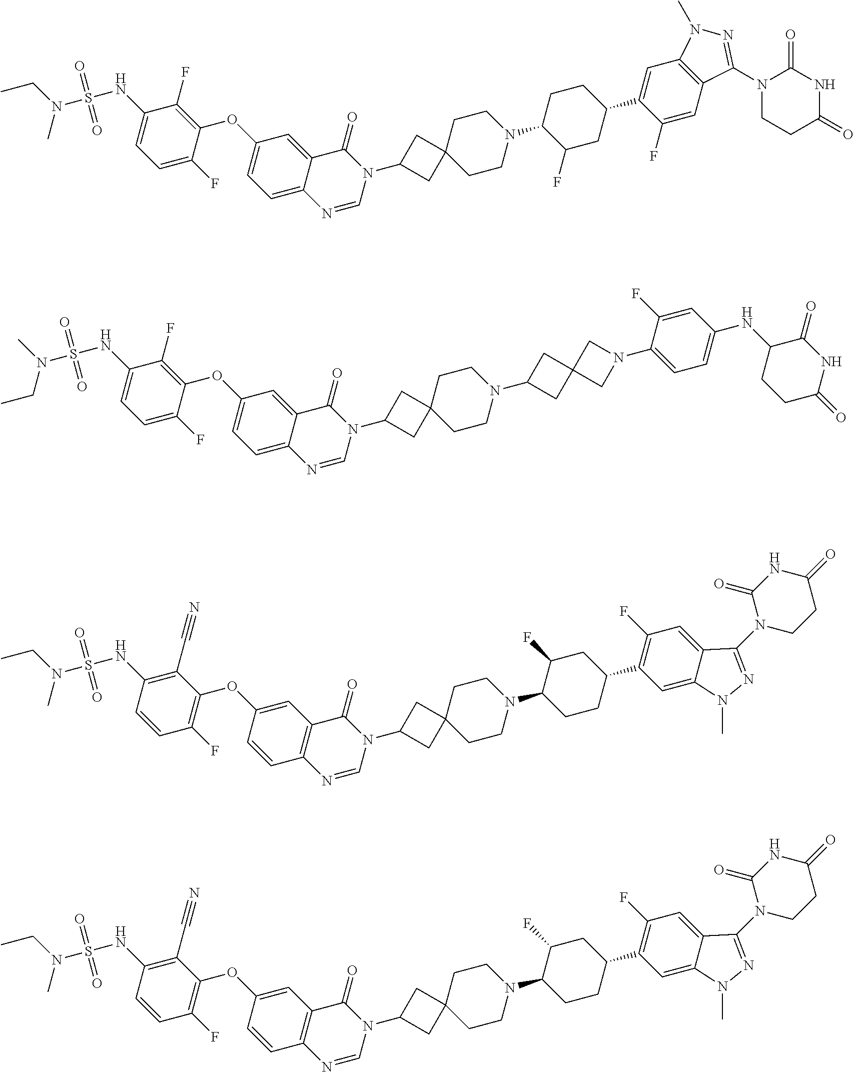 [SMILES: CCN(C)S(=O)(=O)Nc1ccc(F)c(Oc2ccc3ncn(C4CC5(CCN([C@@H]6CC[C@H](c7cc8c(cc7F)c(N7CCC(=O)NC7=O)nn8C)CC6F)CC5)C4)c(=O)c3c2)c1F ; CCN(C)S(=O)(=O)Nc1ccc(F)c(Oc2ccc3ncn(C4CC5(CCN(C6CC7(C6)CN(c6ccc(NC8CCC(=O)NC8=O)cc6F)C7)CC5)C4)c(=O)c3c2)c1F ; CCN(C)S(=O)(=O)Nc1ccc(F)c(Oc2ccc3ncn(C4CC5(CCN([C@@H]6CC[C@@H](c7cc8c(cc7F)c(N7CCC(=O)NC7=O)nn8C)C[C@@H]6F)CC5)C4)c(=O)c3c2)c1C#N ; CCN(C)S(=O)(=O)Nc1ccc(F)c(Oc2ccc3ncn(C4CC5(CCN([C@@H]6CC[C@@H](c7cc8c(cc7F)c(N7CCC(=O)NC7=O)nn8C)C[C@H]6F)CC5)C4)c(=O)c3c2)c1C#N]

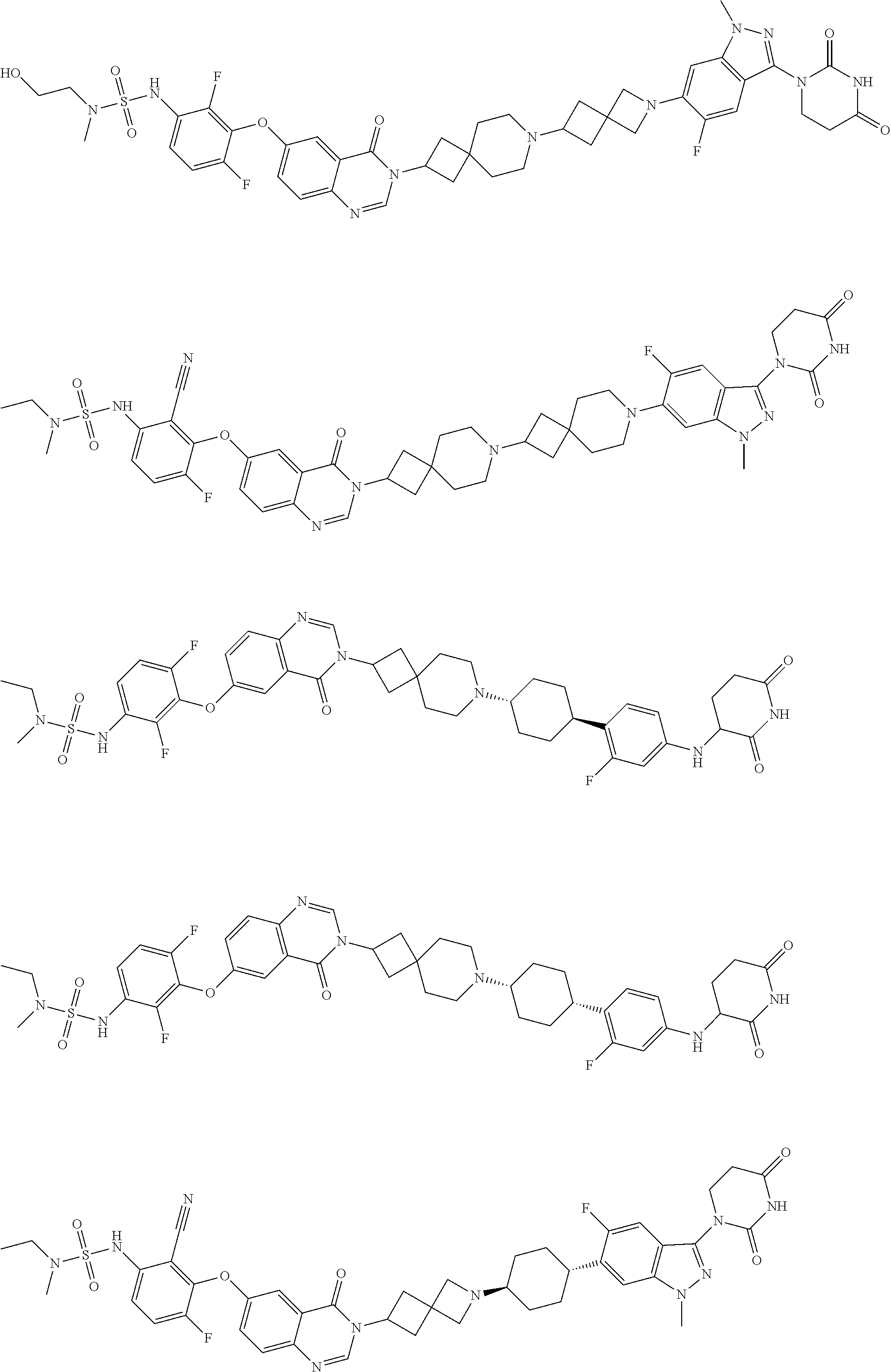

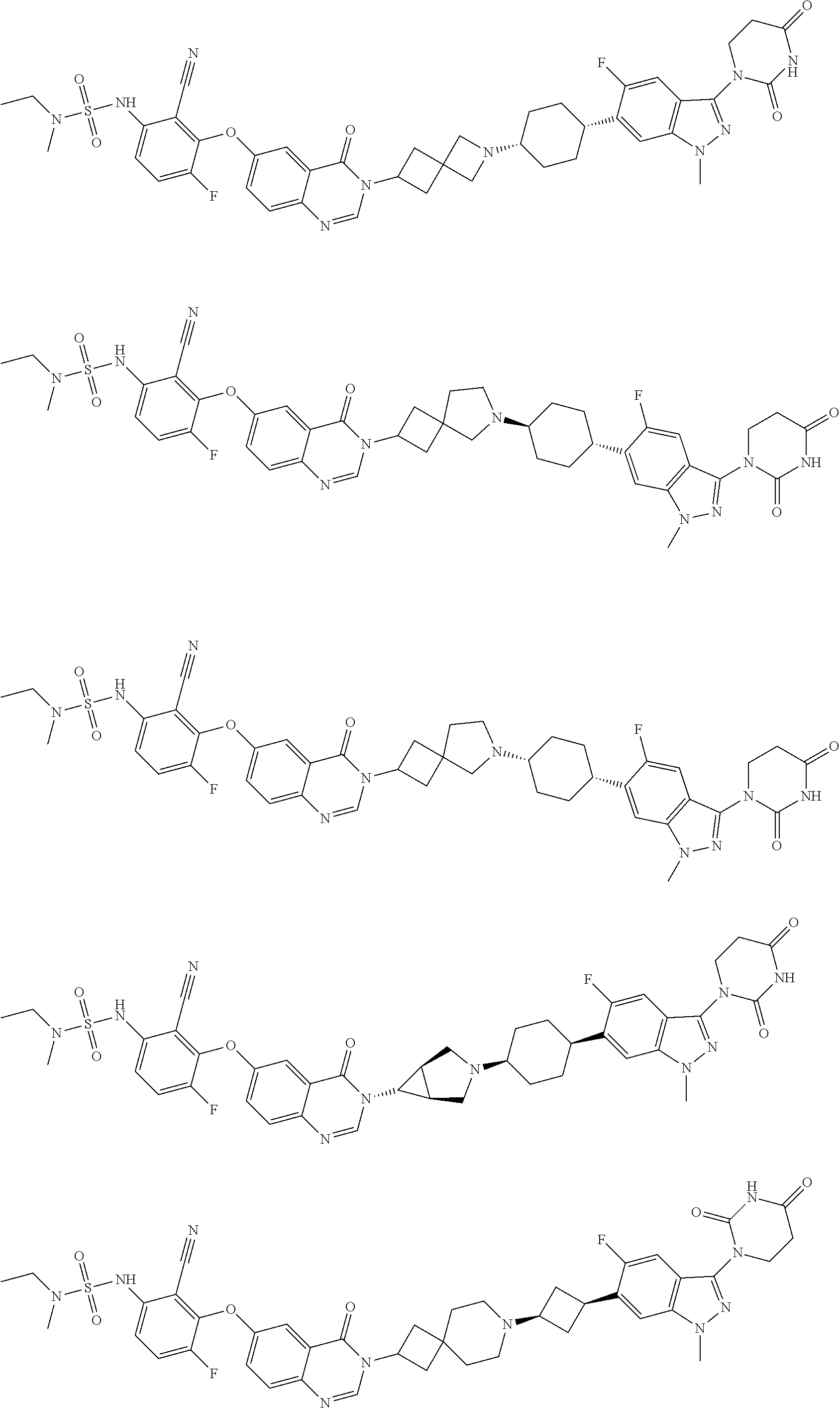

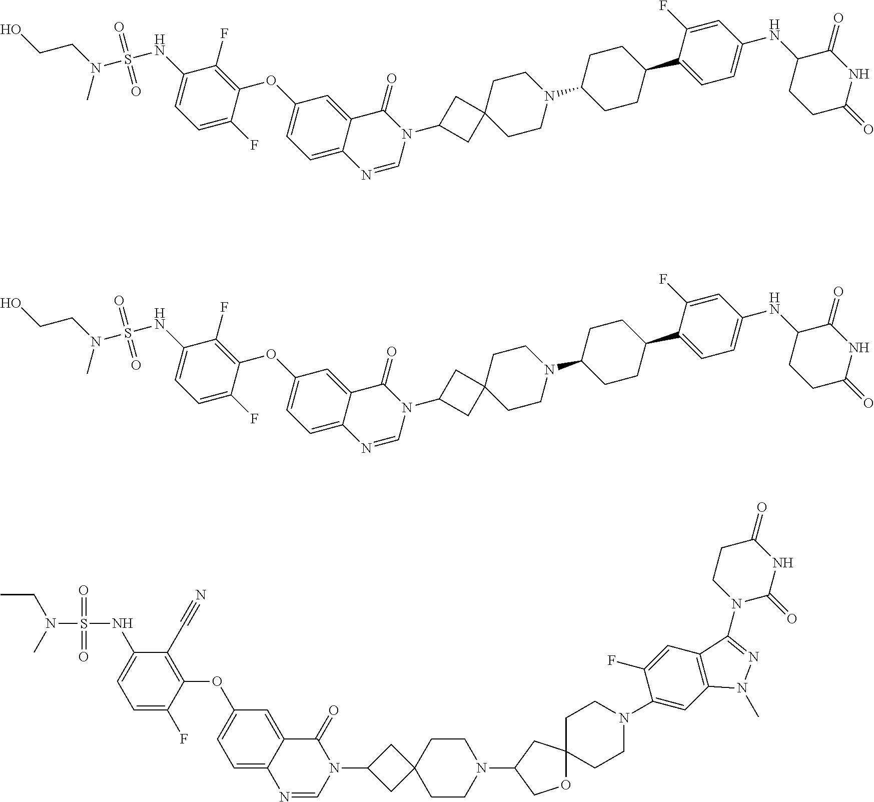 [SMILES: CN(CCO)S(=O)(=O)Nc1ccc(F)c(Oc2ccc3ncn(C4CC5(CCN([C@H]6CC[C@H](c7ccc(NC8CCC(=O)NC8=O)cc7F)CC6)CC5)C4)c(=O)c3c2)c1F ; CN(CCO)S(=O)(=O)Nc1ccc(F)c(Oc2ccc3ncn(C4CC5(CCN([C@H]6CC[C@@H](c7ccc(NC8CCC(=O)NC8=O)cc7F)CC6)CC5)C4)c(=O)c3c2)c1F ; CCN(C)S(=O)(=O)Nc1ccc(F)c(Oc2ccc3ncn(C4CC5(CCN(C6COC7(CCN(c8cc9c(cc8F)c(N8CCC(=O)NC8=O)nn9C)CC7)C6)CC5)C4)c(=O)c3c2)c1C#N]

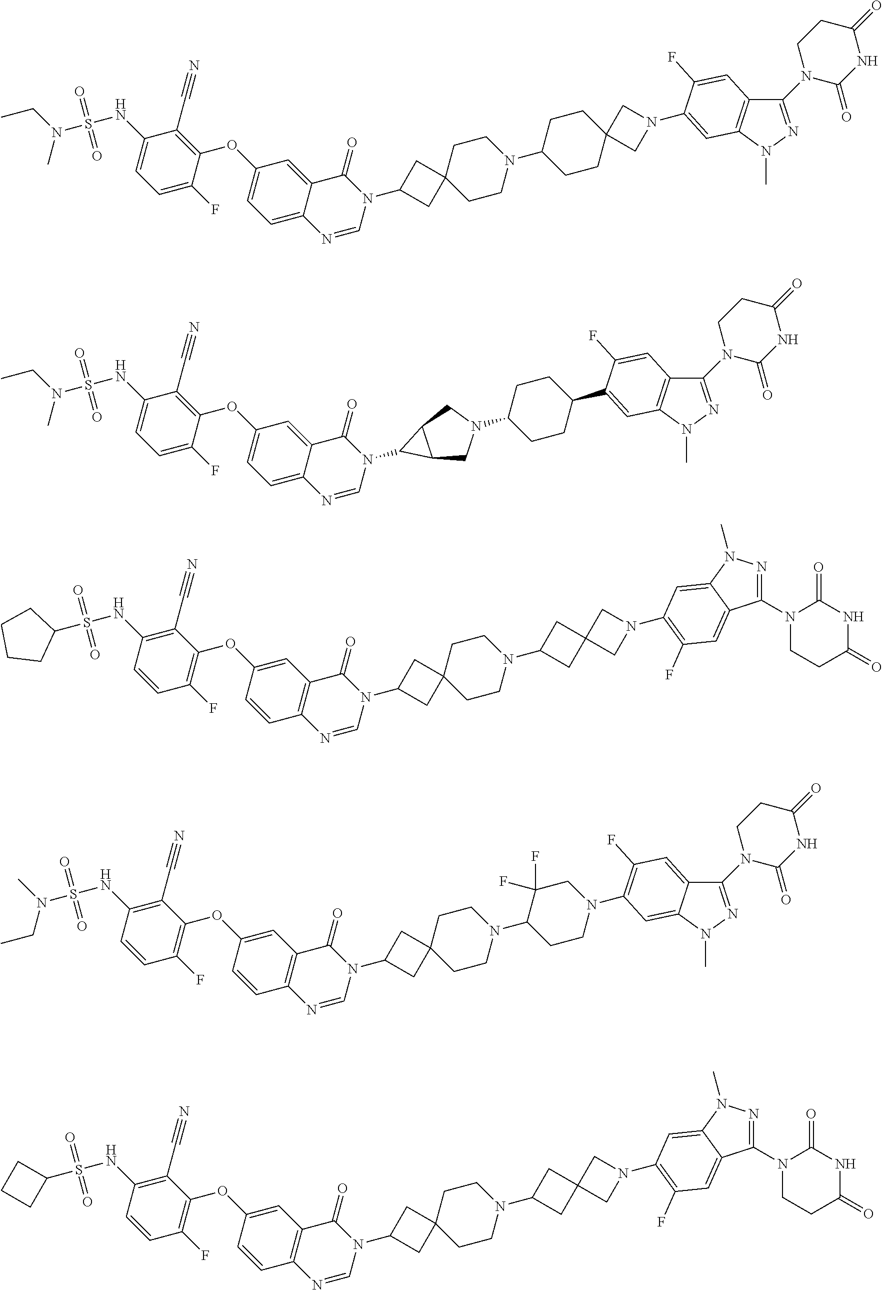

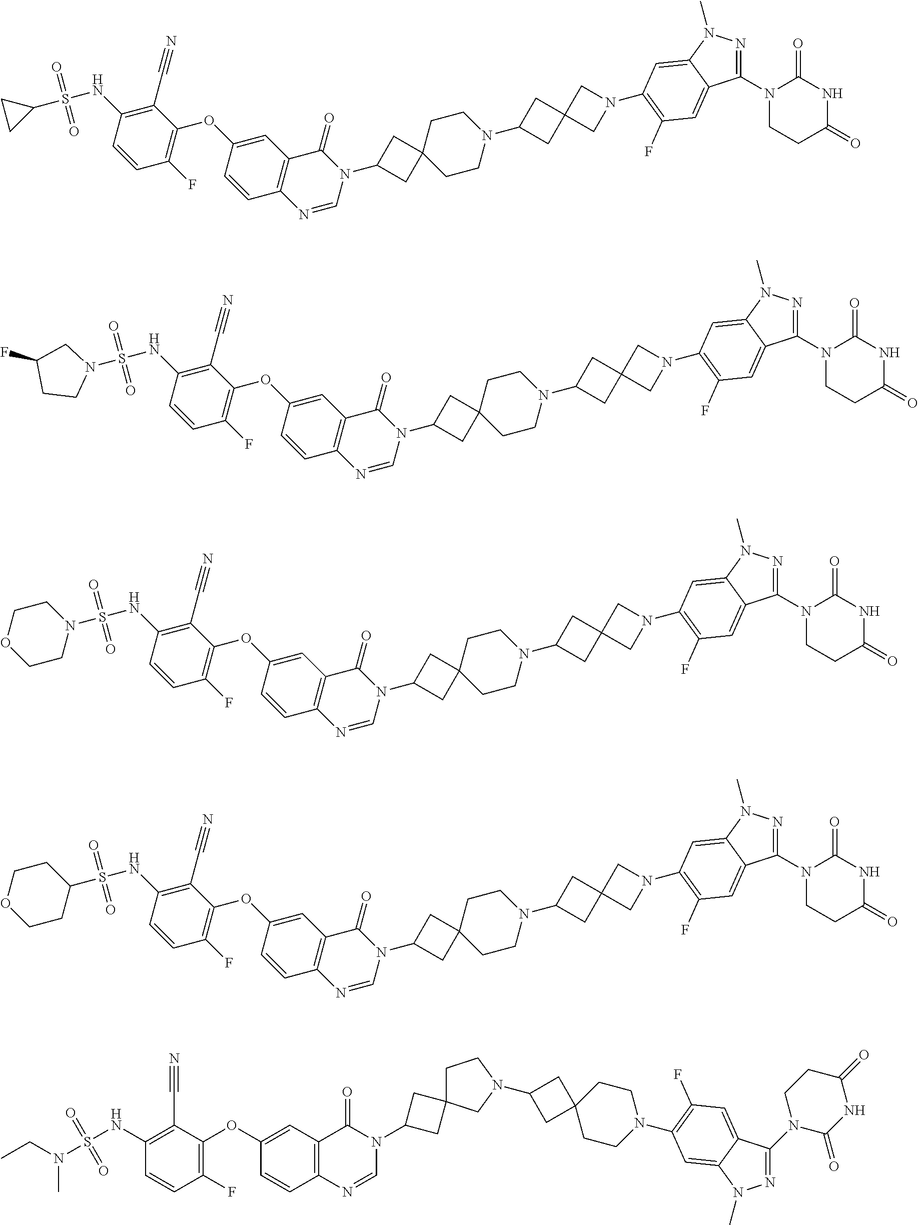

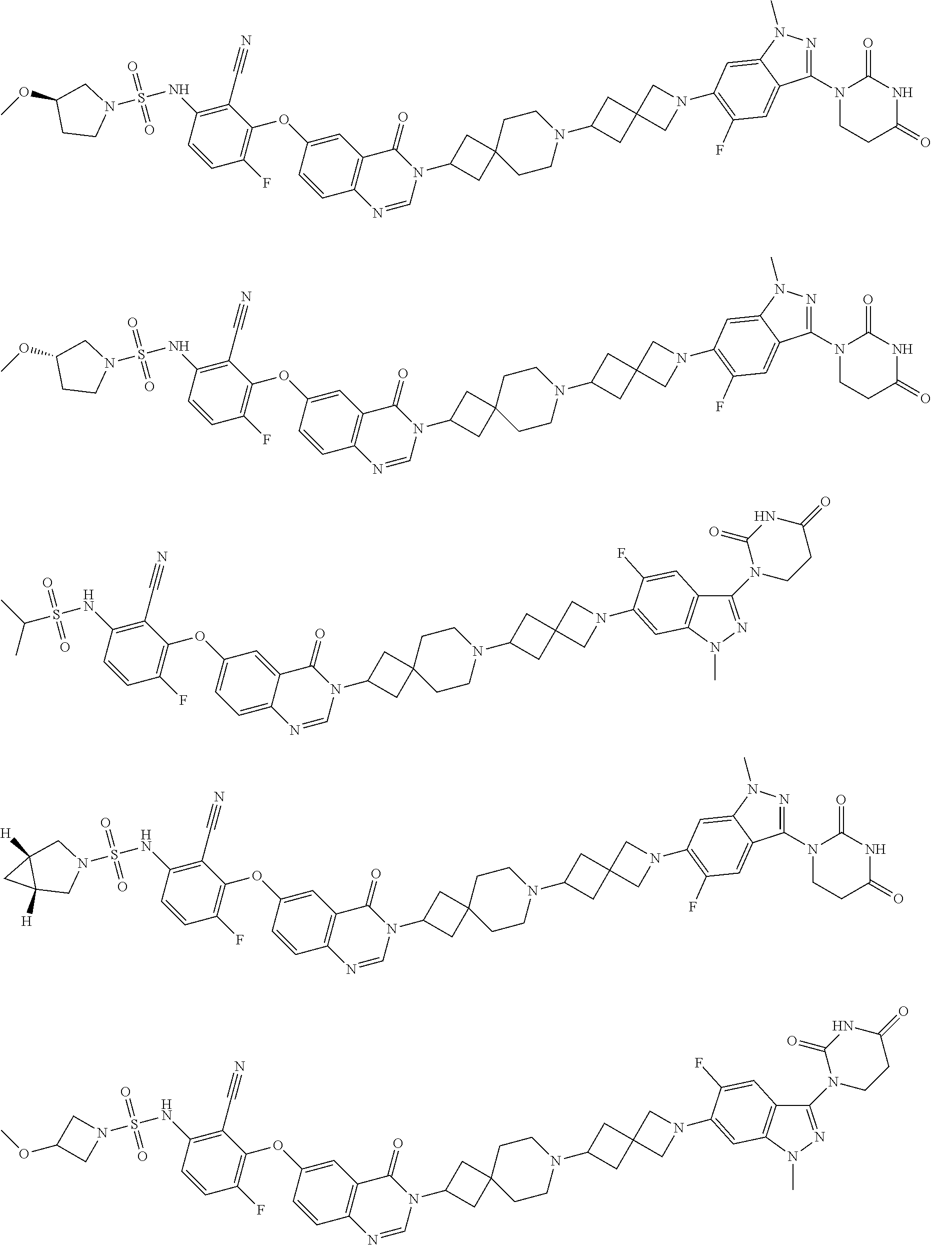

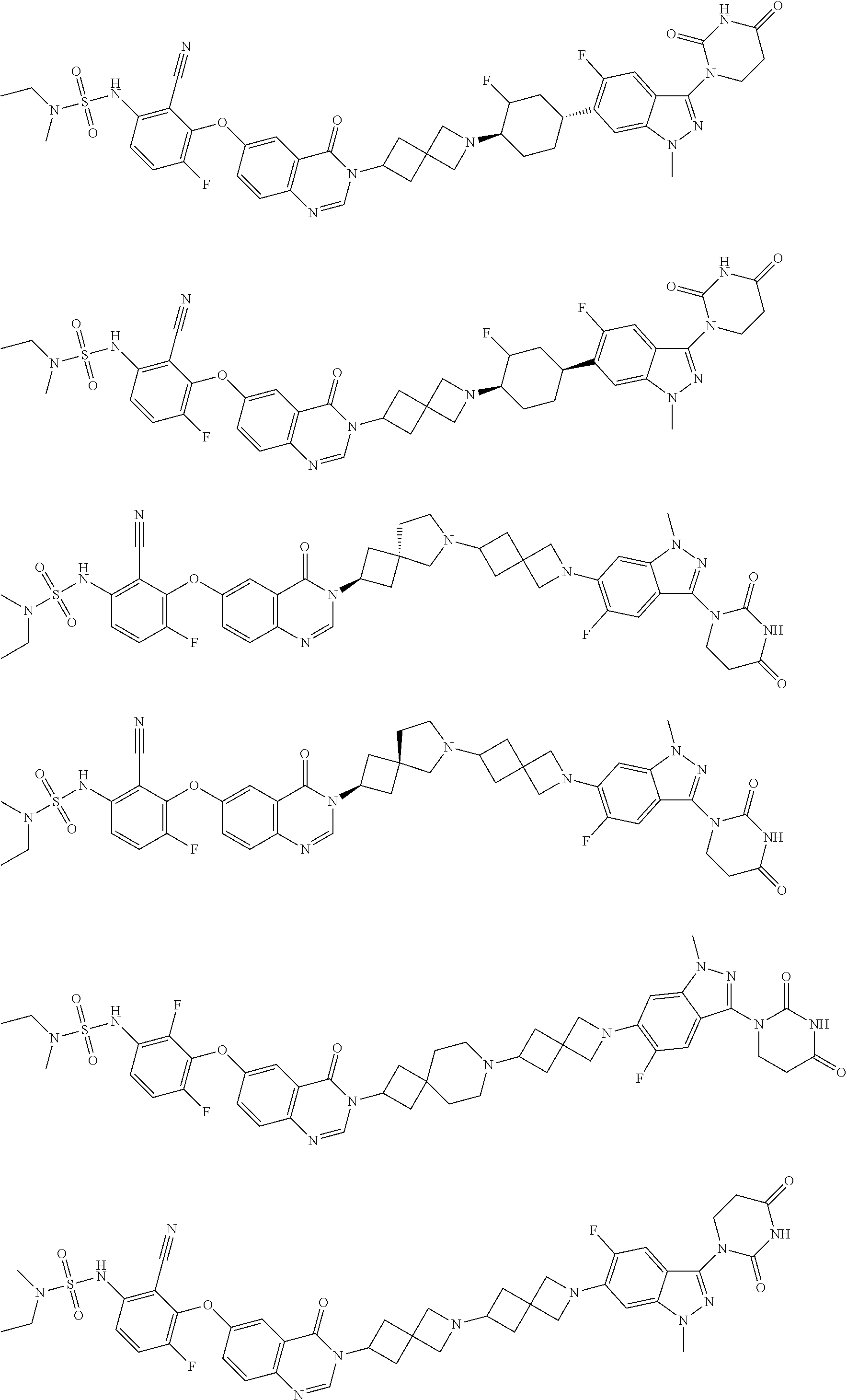

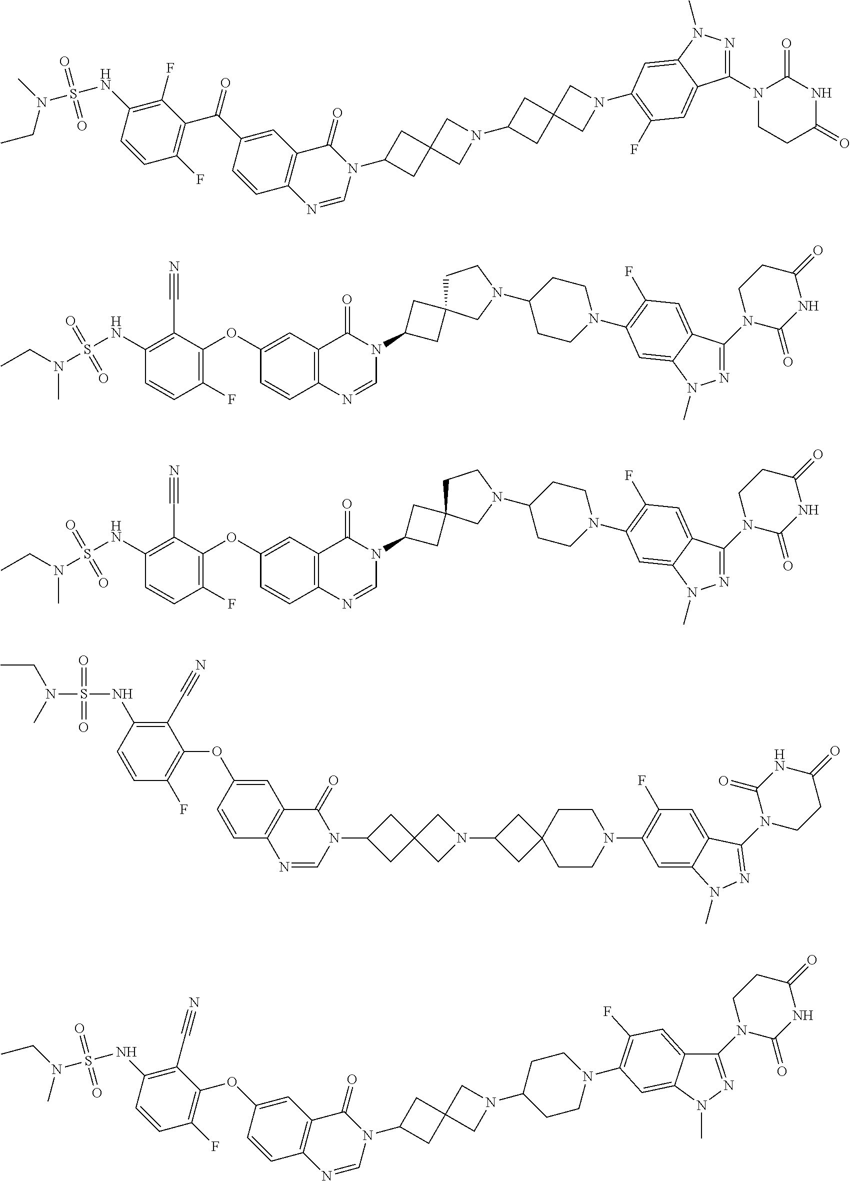

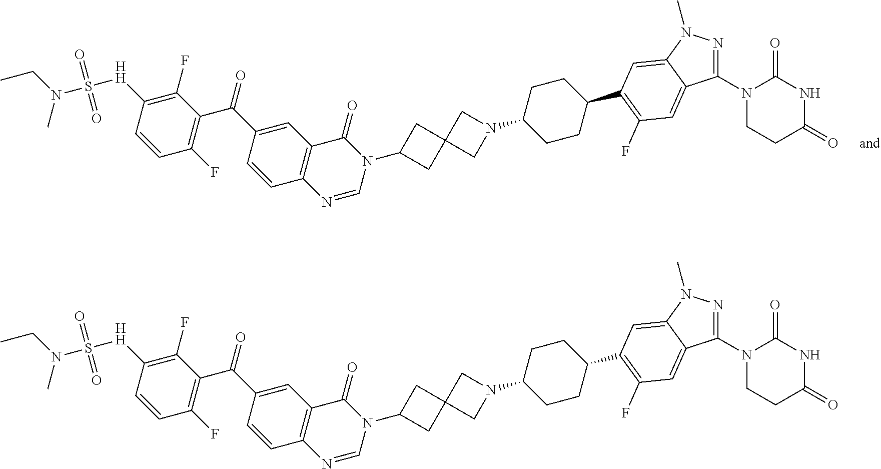

or a pharmaceutically acceptable salt thereof.

The invention further provides the following.

A compound of Formula II or Formula XVIII or a pharmaceutically acceptable salt thereof, for use as a therapeutically active substance in the treatment of a mutant BRAF mediated disorder.

A pharmaceutical composition comprising a compound of Formula II or Formula XVIII or a pharmaceutically acceptable salt thereof, and a pharmaceutically acceptable carrier.

The use of a compound of Formula II or Formula XVIII or a pharmaceutically acceptable salt thereof, for the therapeutic and/or prophylactic treatment of cancer.

A compound of Formula II or Formula XVIII or a pharmaceutically acceptable salt thereof, for use in the treatment and/or prophylaxis of cancer.

The use of a compound of Formula II or Formula XVIII or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula II or Formula XVIII or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula III and Formula XIX

In certain embodiments a compound of the invention is selected from the compound of Formula III and the compound of Formula XIX:

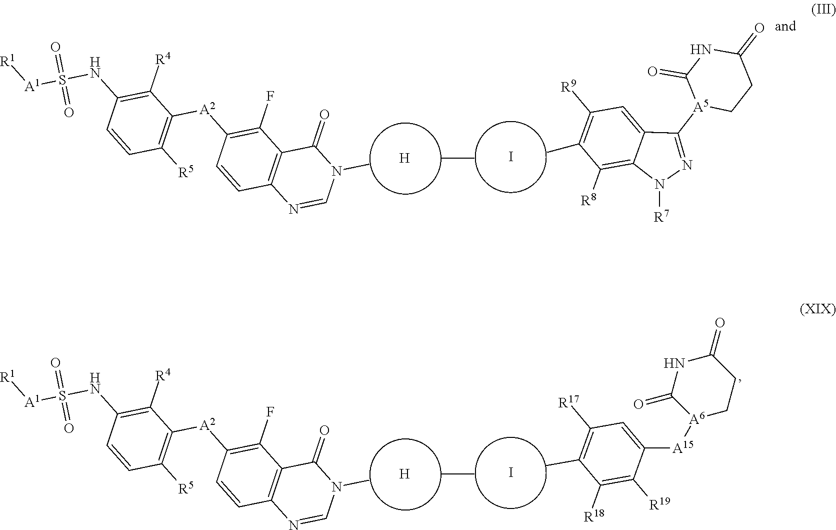

or a pharmaceutically acceptable salt thereof,
wherein
Cycle \textcircled{H} is a spiroheterocyclyl containing 1 oxygen atom and 0 or 1 nitrogen atom and containing no other heteroatoms, wherein cycle \textcircled{H} is optionally substituted with one or two substituents independently selected from halogen, alkyl and alkoxy;
Cycle \textcircled{I} is a cycloalkyl, wherein cycle \textcircled{I} is optionally substituted with one or two substituents independently selected from halogen, alkyl and alkoxy; and all other substituents are as defined herein.

In certain embodiments the compound of Formula III or Formula XIX is selected from:

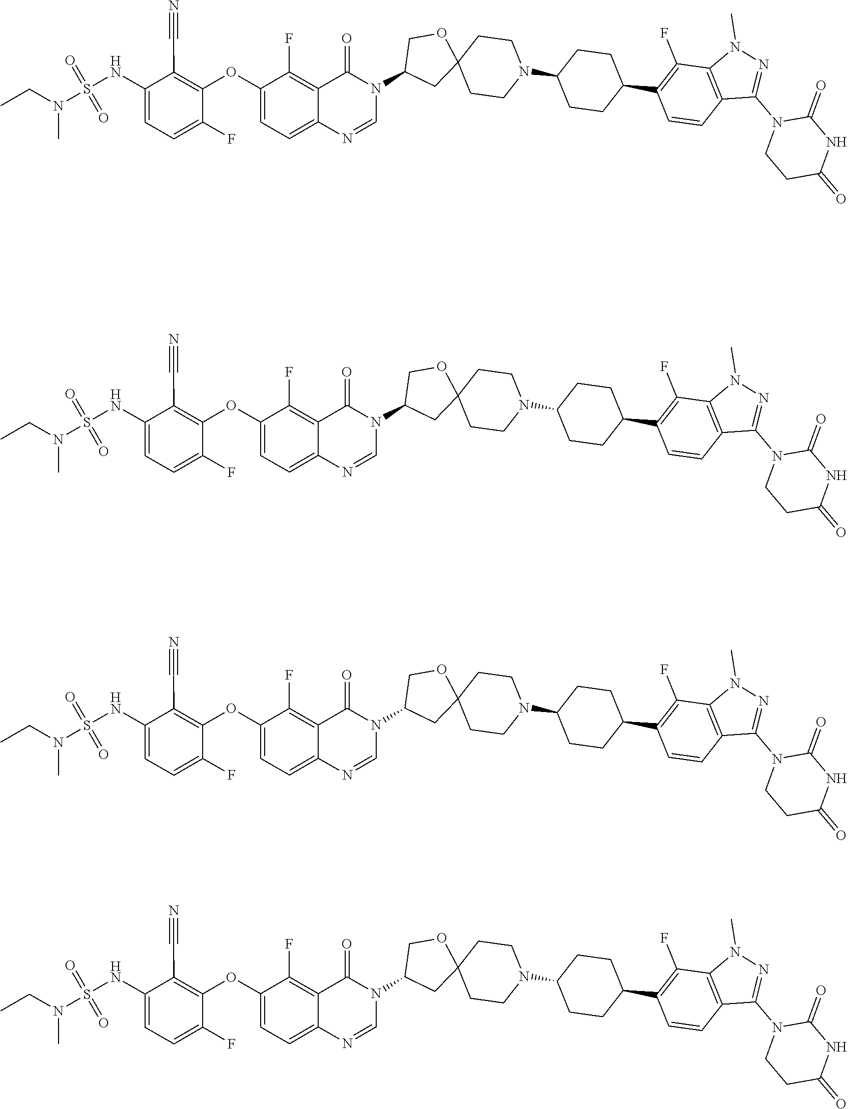

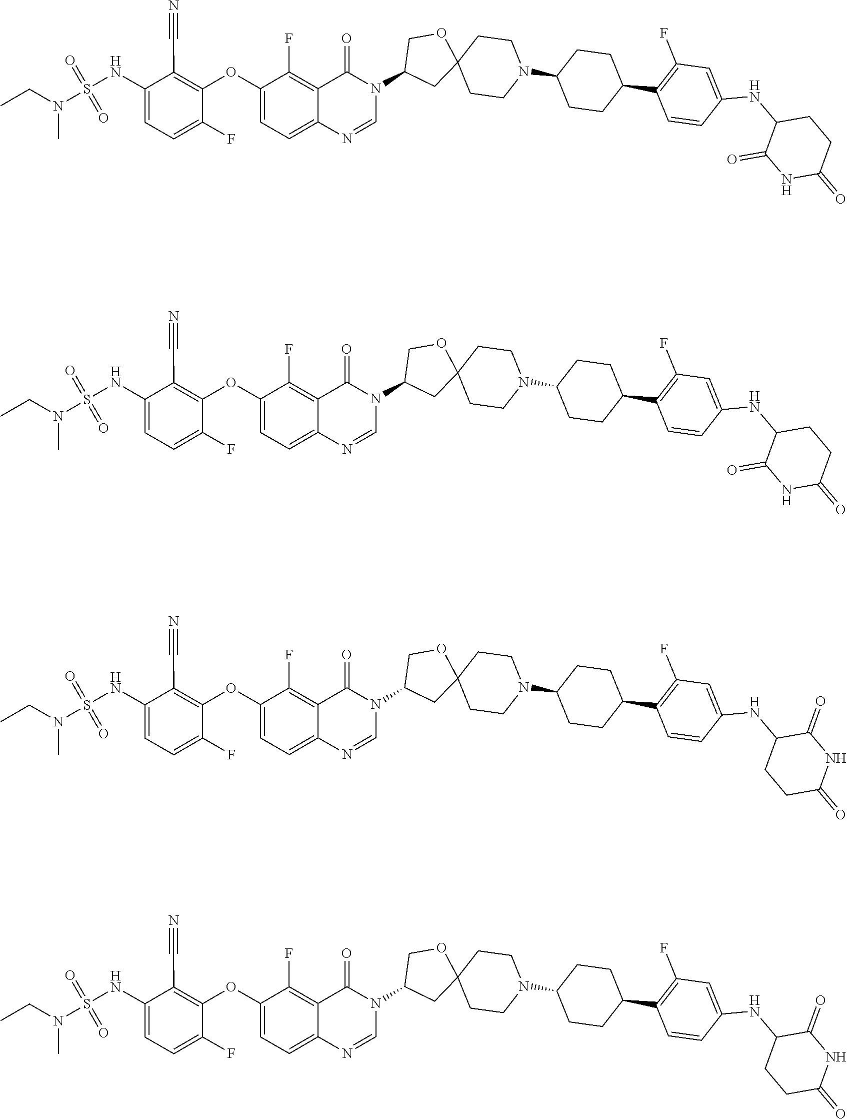

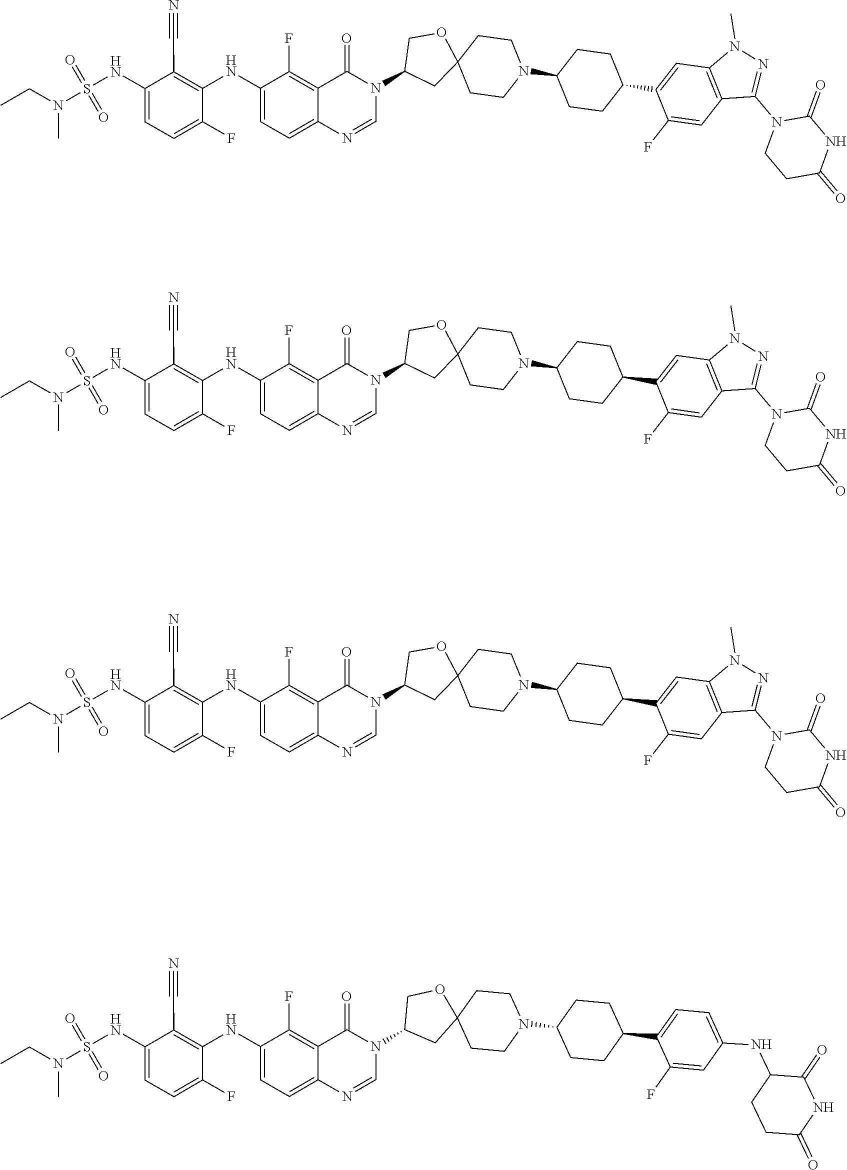

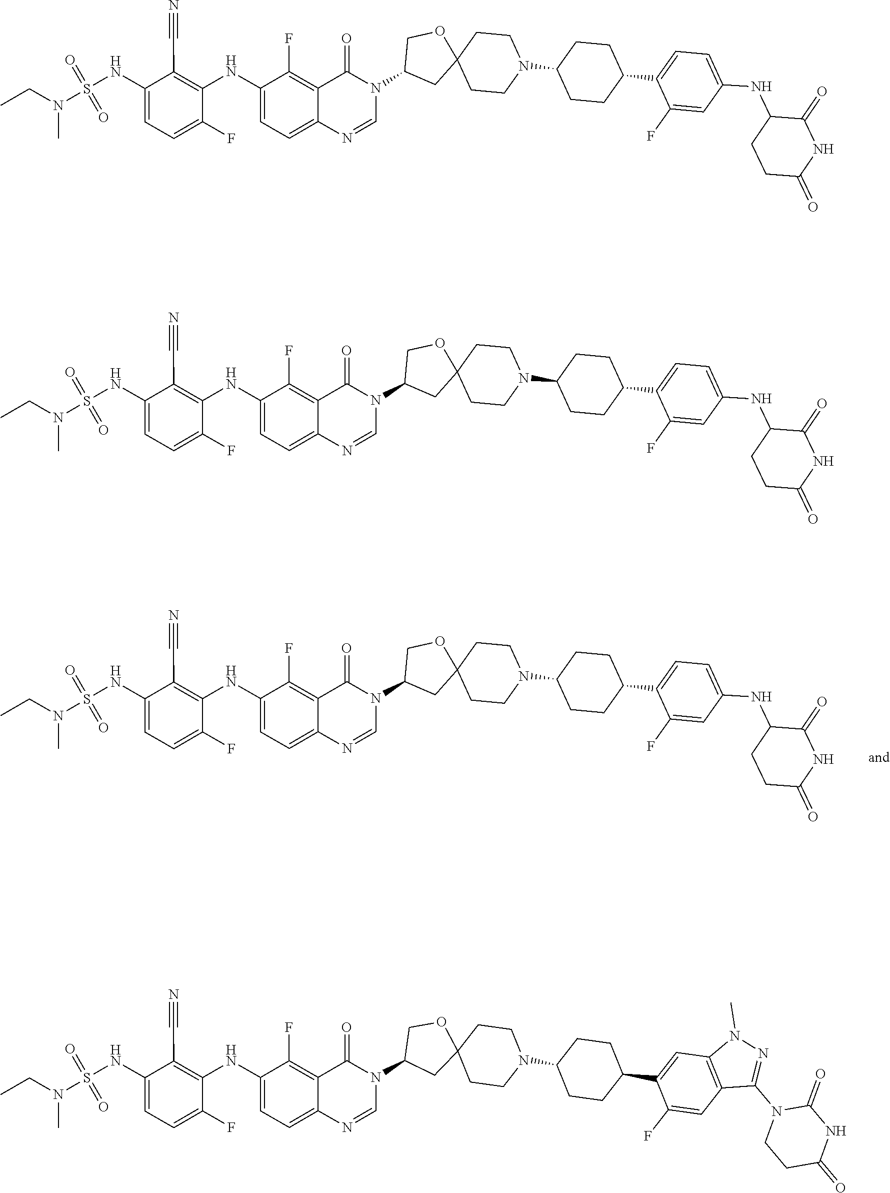

or a pharmaceutically acceptable salt thereof.

The invention further provides the following.

A compound of Formula III or Formula XIX or a pharmaceutically acceptable salt thereof, for use as a therapeutically active substance in the treatment of a mutant BRAF mediated disorder.

A pharmaceutical composition comprising a compound of Formula III or Formula XIX or a pharmaceutically acceptable salt thereof, and a pharmaceutically acceptable carrier.

The use of a compound of Formula III or Formula XIX or a pharmaceutically acceptable salt thereof, for the therapeutic and/or prophylactic treatment of cancer.

A compound of Formula III or Formula XIX or a pharmaceutically acceptable salt thereof, for use in the treatment and/or prophylaxis of cancer.

The use of a compound of Formula III or Formula XIX or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula III or Formula XIX or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula IV and Formula XX

In certain embodiments a compound of the invention is selected from the compound of Formula IV and the compound of Formula XX:

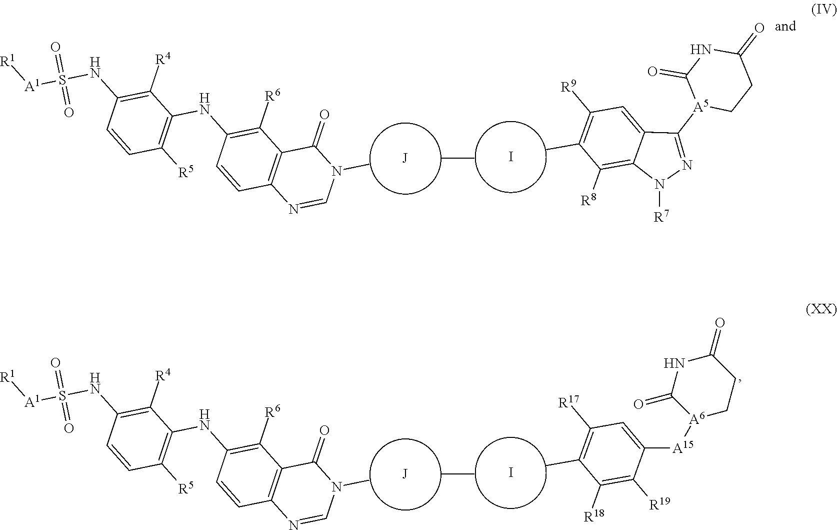

or a pharmaceutically acceptable salt thereof,
wherein
Cycle \textcircled{J} is a spiroheterocyclyl containing 1 or 2 nitrogen atoms and 0 or 1 oxygen atoms and containing no other heteroatoms, wherein cycle \textcircled{J} is optionally substituted with one or two substituents independently selected from halogen, alkyl and alkoxy;
Cycle \textcircled{I} is a cycloalkyl, wherein cycle \textcircled{I} is optionally substituted with one or two substituents independently selected from halogen, alkyl and alkoxy; and all other substituents are as defined herein.

In certain embodiments the compound of Formula IV or Formula XX is selected from:

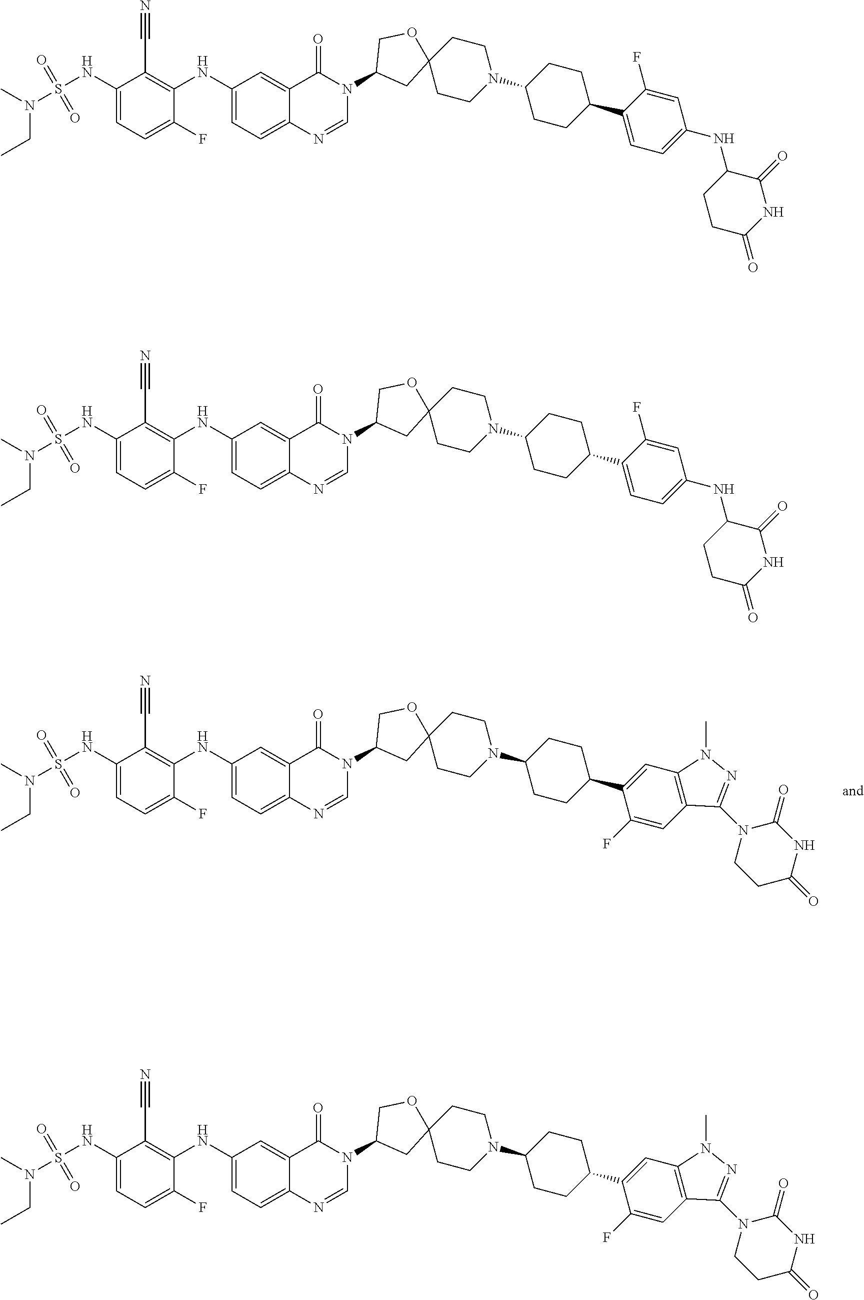

or a pharmaceutically acceptable salt thereof

The invention further provides the following.

A compound of Formula IV or Formula XX or a pharmaceutically acceptable salt thereof, for use as a therapeutically active substance in the treatment of a mutant BRAF mediated disorder.

A pharmaceutical composition comprising a compound of Formula IV or Formula XX or a pharmaceutically acceptable salt thereof, and a pharmaceutically acceptable carrier.

The use of a compound of Formula IV or Formula XX or a pharmaceutically acceptable salt thereof, for the therapeutic and/or prophylactic treatment of cancer.

A compound of Formula IV or Formula XX or a pharmaceutically acceptable salt thereof, for use in the treatment and/or prophylaxis of cancer.

The use of a compound of Formula IV or Formula XX or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula IV or Formula XX or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula V and Formula XXI

In certain embodiments a compound of the invention is selected from the compound of Formula V and the compound of Formula XXI:

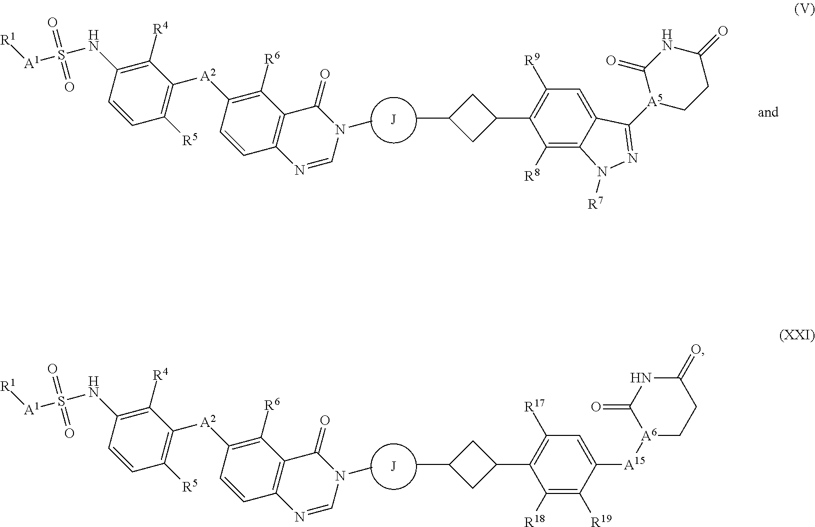

or a pharmaceutically acceptable salt thereof,
wherein:
Cycle \textcircled{J} is a spiroheterocyclyl containing 1 or 2 nitrogen atoms and 0 or 1 oxygen atom and containing no other heteroatoms, wherein cycle \textcircled{J} is optionally substituted with one or two substituents independently selected from halogen, alkyl and alkoxy; and all other substituents are as defined herein.

In certain embodiments the compound of Formula V or Formula XXI is selected from:

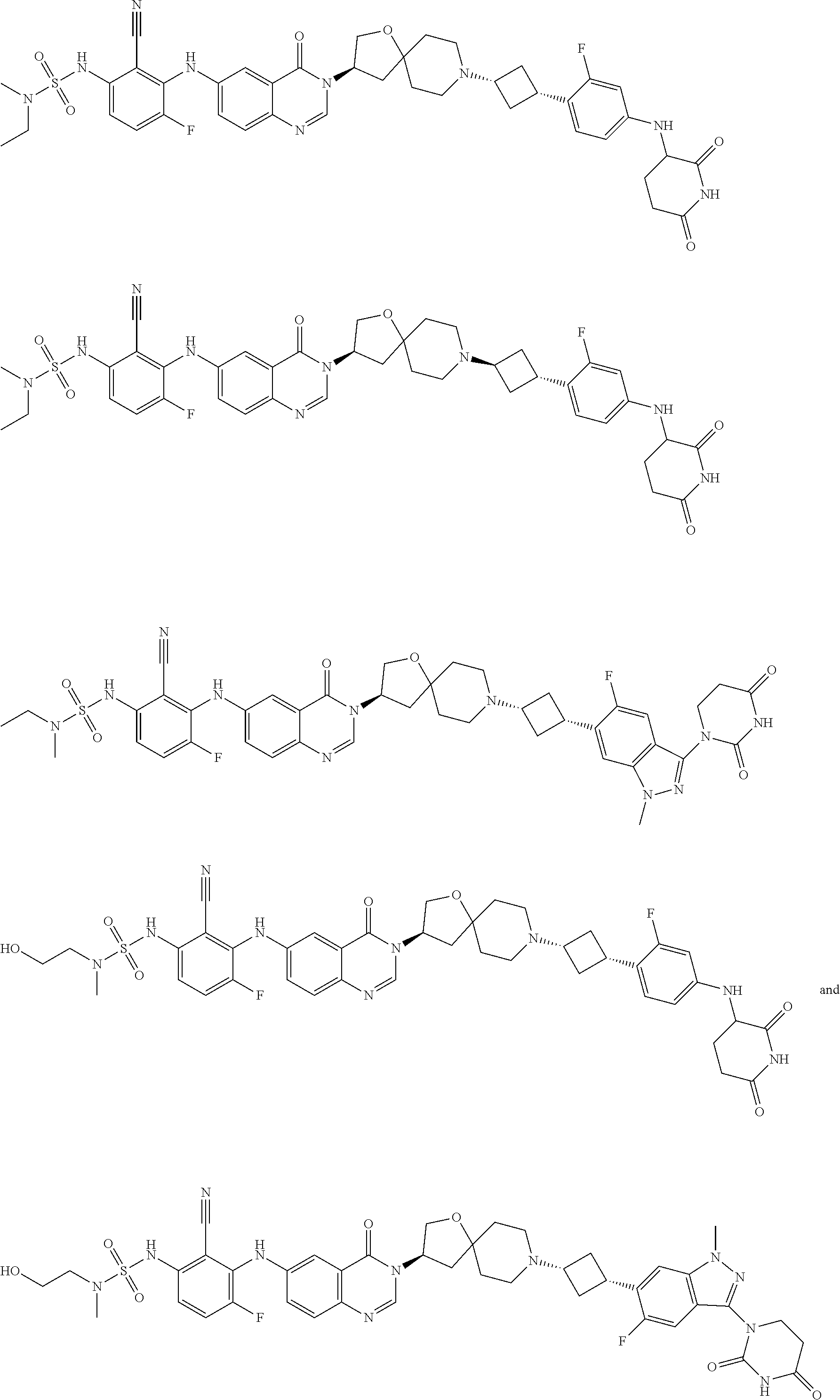

or a pharmaceutically acceptable salt thereof.

The invention further provides the following.

A compound of Formula V or Formula XXI or a pharmaceutically acceptable salt thereof, for use as a therapeutically active substance in the treatment of a mutant BRAF mediated disorder.

A pharmaceutical composition comprising a compound of Formula V or Formula XXI or a pharmaceutically acceptable salt thereof, and a pharmaceutically acceptable carrier.

The use of a compound of Formula V or Formula XXI or a pharmaceutically acceptable salt thereof, for the therapeutic and/or prophylactic treatment of cancer.

A compound of Formula V or Formula XXI or a pharmaceutically acceptable salt thereof, for use in the treatment and/or prophylaxis of cancer.

The use of a compound of Formula V or Formula XXI or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula V or Formula XXI or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula VI and Formula XXII

In certain embodiments a compound of the invention is selected from the compound of Formula VI and the compound of Formula XXII:

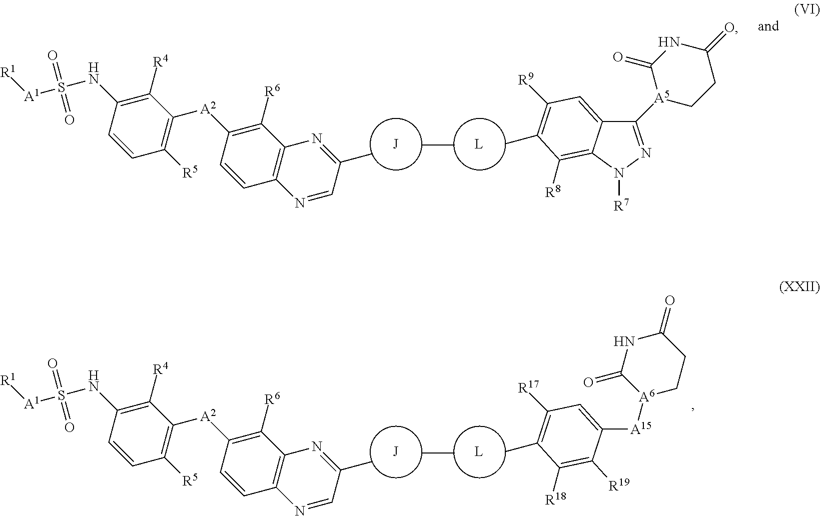

or a pharmaceutically acceptable salt thereof,
wherein
Cycle \textcircled{J} is a spiroheterocyclyl containing 1 or 2 nitrogen atoms and 0 or 1 oxygen atom and containing no other heteroatoms, wherein cycle \textcircled{J} is optionally substituted with one or two substituents independently selected from halogen, alkyl and alkoxy;
Cycle \textcircled{L} is a cycloalkyl, piperidine-1,4-diyl, spiroheterocyclyl containing 1 or 2 nitrogen atoms and containing no other heteroatoms, wherein cycle \textcircled{L} is optionally substituted with one or two substituents independently selected from halogen, alkyl and alkoxy; and all other substituents are as defined herein.

In certain embodiments the compound of Formula VI or Formula XXII is selected from:

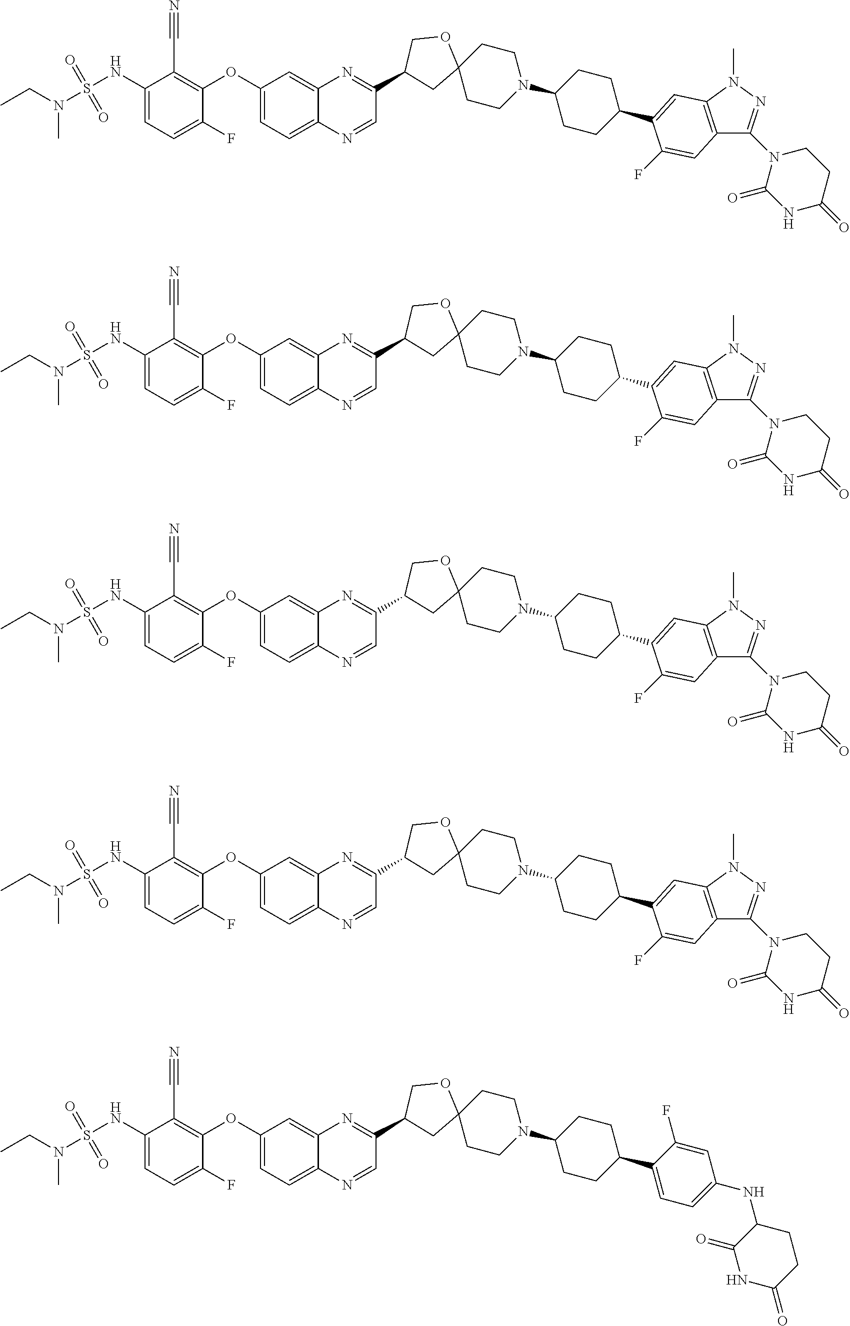

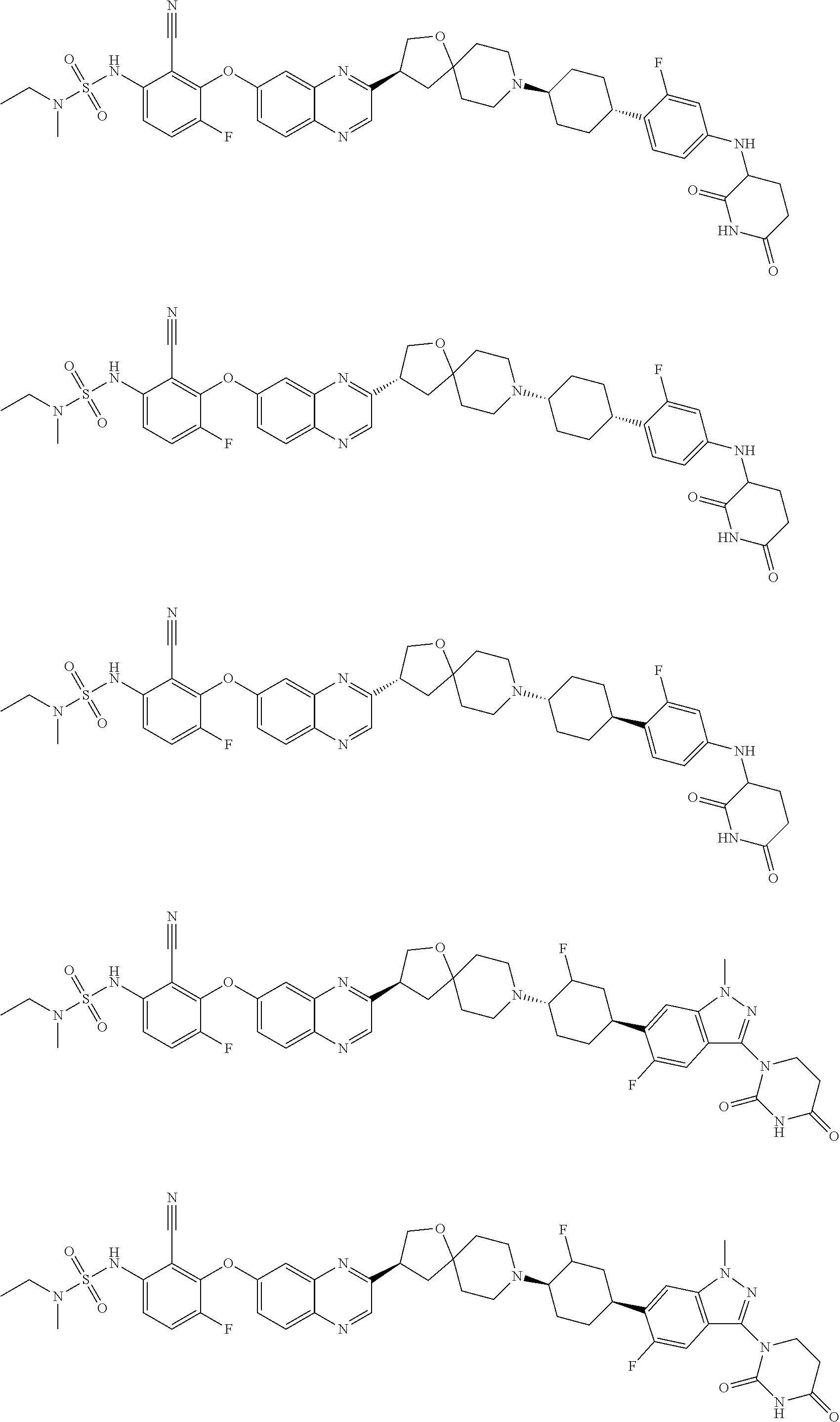

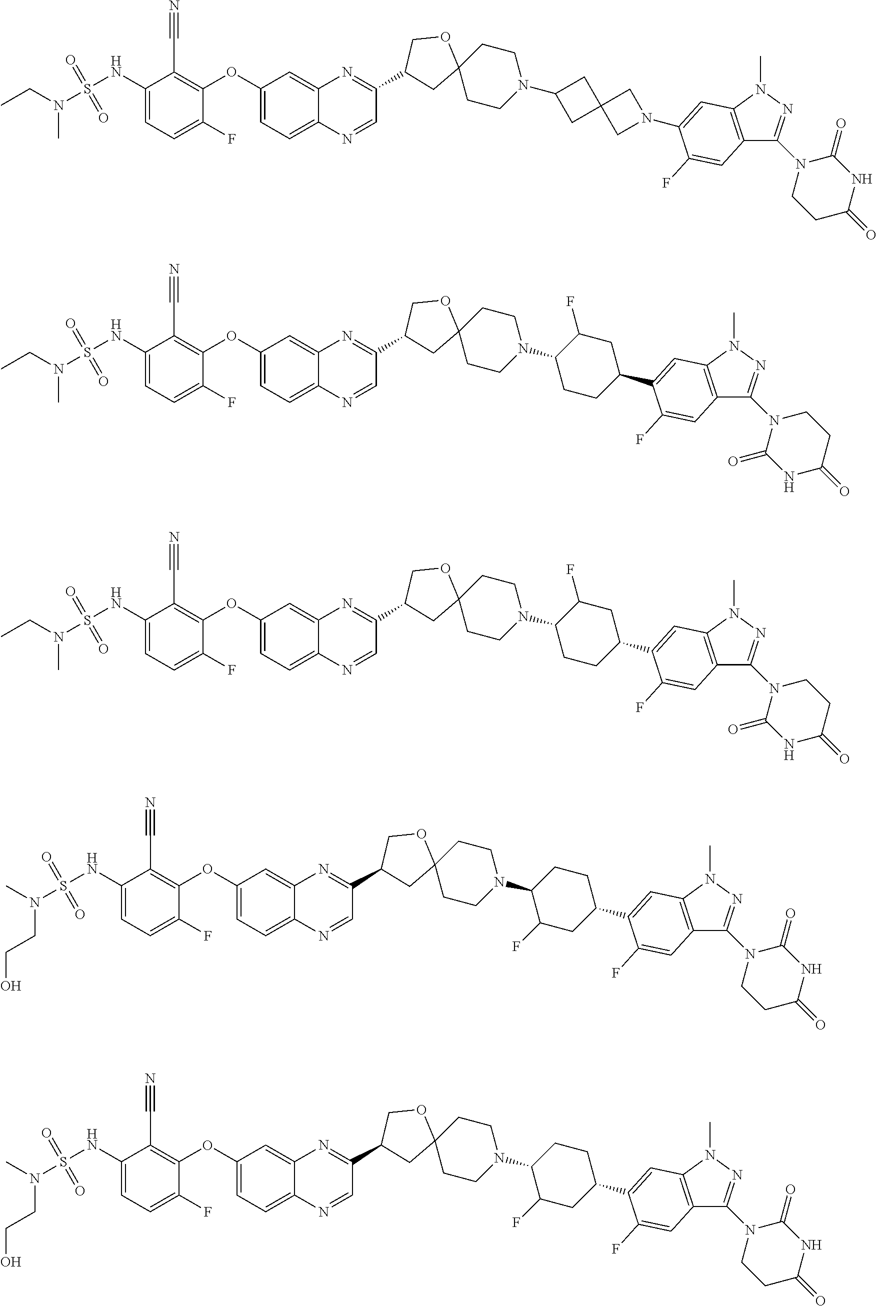

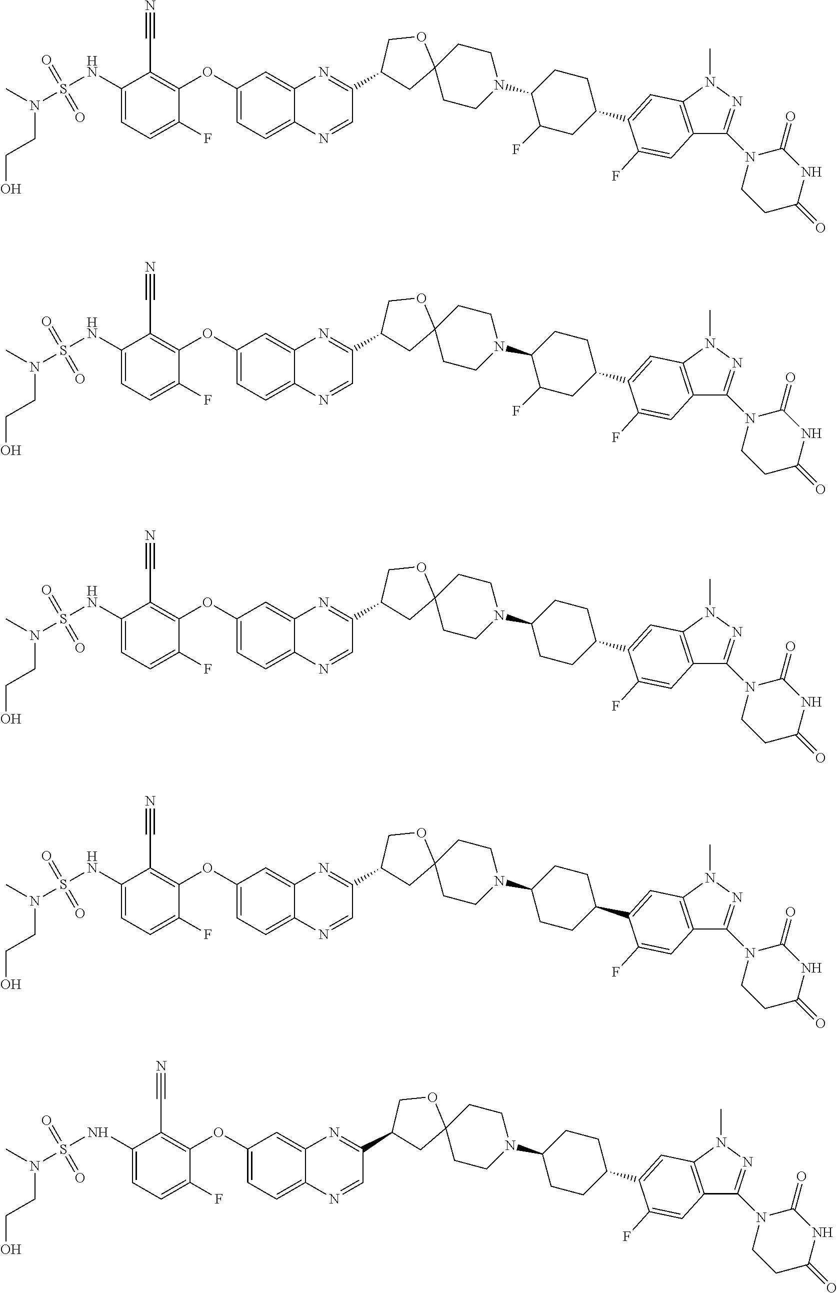

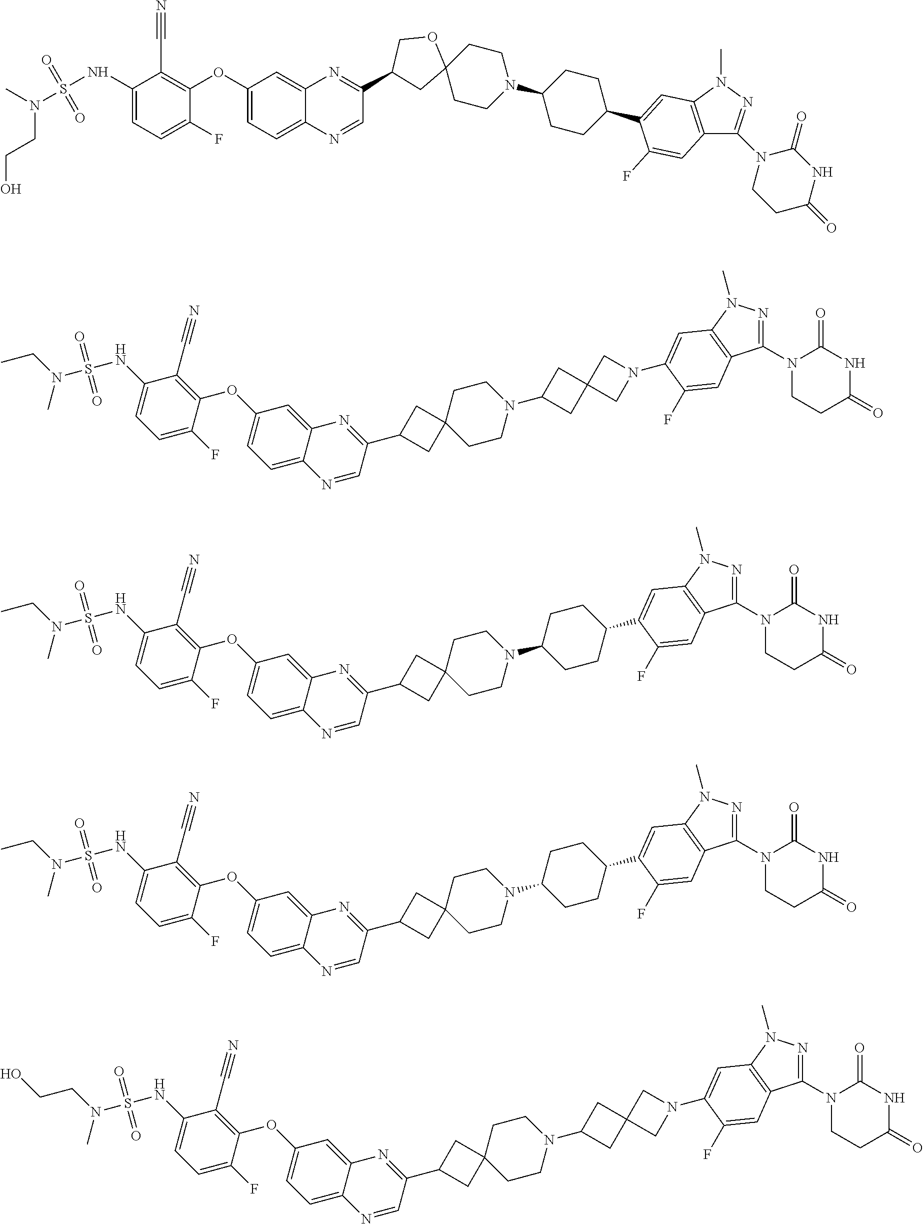

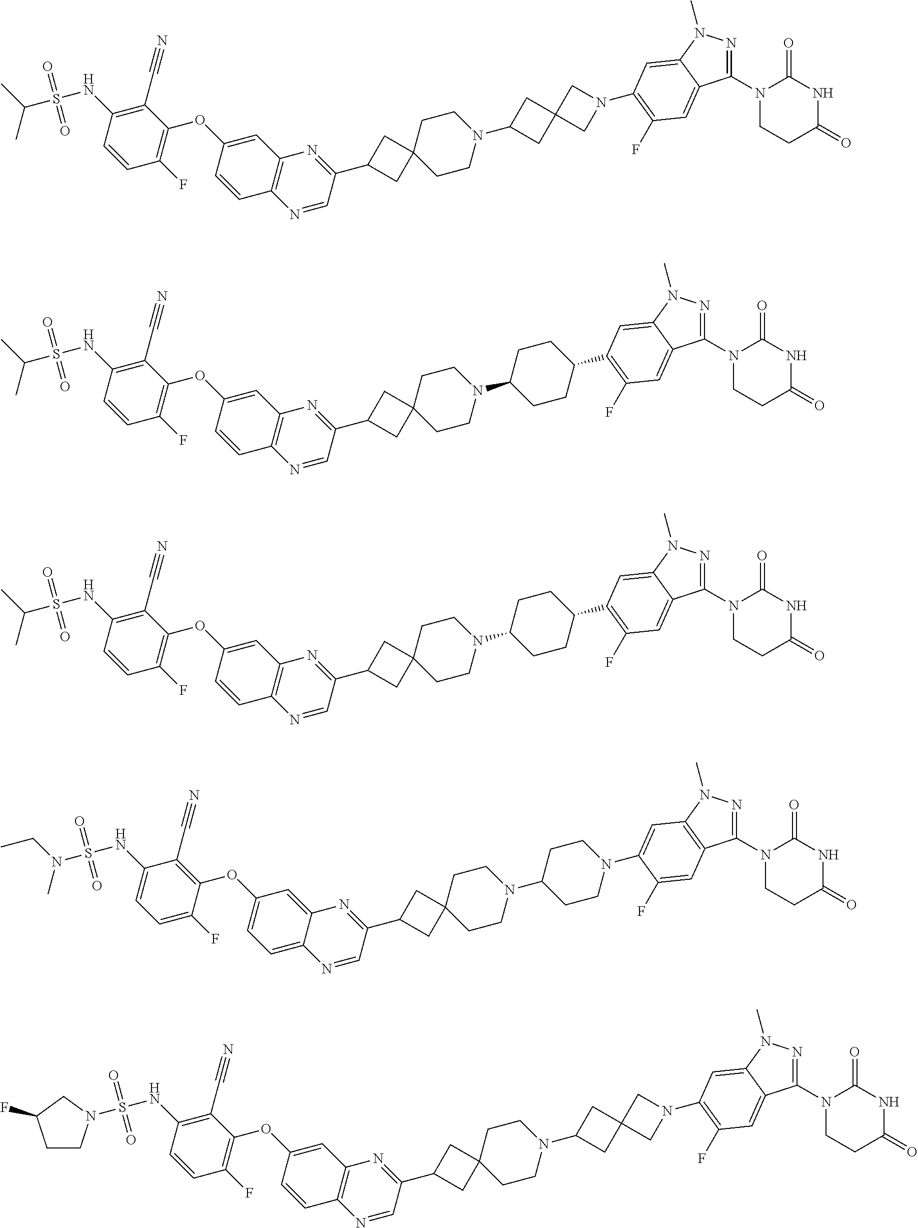

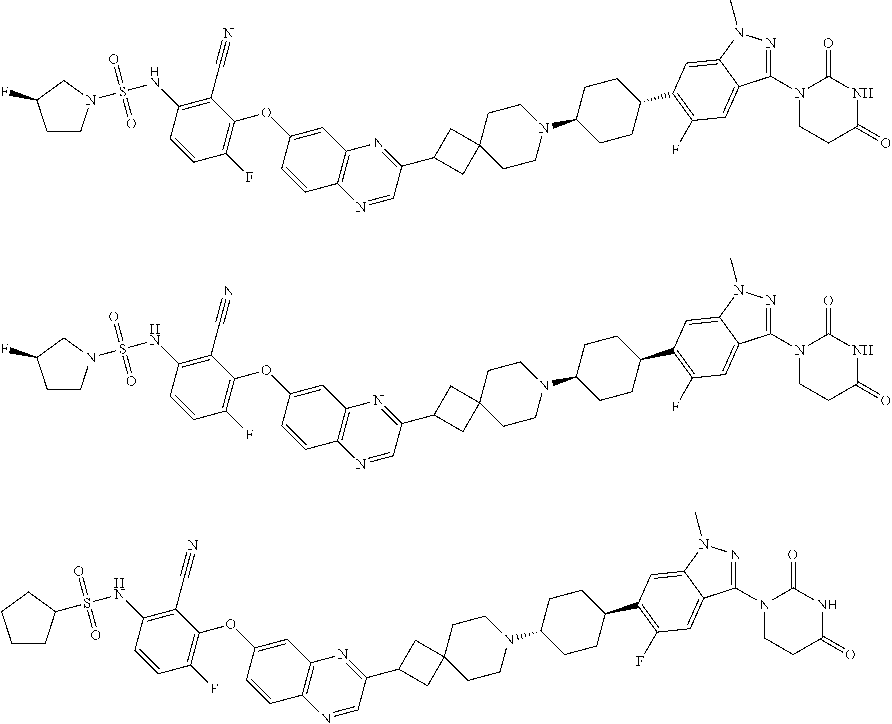 [SMILES: Cn1nc(N2CCC(=O)NC2=O)c2cc(F)c([C@H]3CC[C@H](N4CCC5(CC4)CC(c4cnc6ccc(Oc7c(F)ccc(NS(=O)(=O)N8CC[C@@H](F)C8)c7C#N)cc6n4)C5)CC3)cc21 ; Cn1nc(N2CCC(=O)NC2=O)c2cc(F)c([C@H]3CC[C@@H](N4CCC5(CC4)CC(c4cnc6ccc(Oc7c(F)ccc(NS(=O)(=O)N8CC[C@@H](F)C8)c7C#N)cc6n4)C5)CC3)cc21 ; Cn1nc(N2CCC(=O)NC2=O)c2cc(F)c([C@H]3CC[C@H](N4CCC5(CC4)CC(c4cnc6ccc(Oc7c(F)ccc(NS(=O)(=O)C8CCCC8)c7C#N)cc6n4)C5)CC3)cc21]

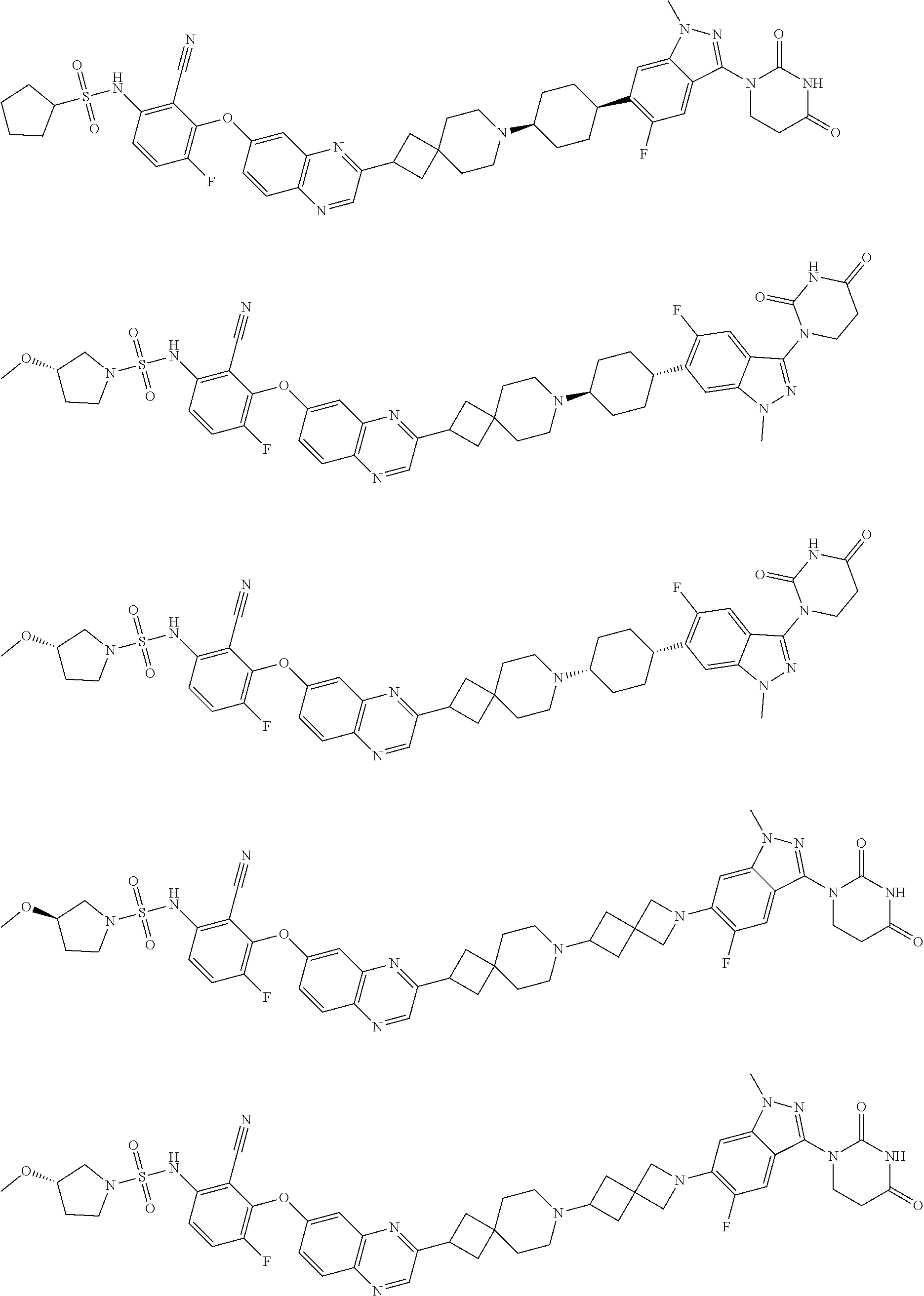

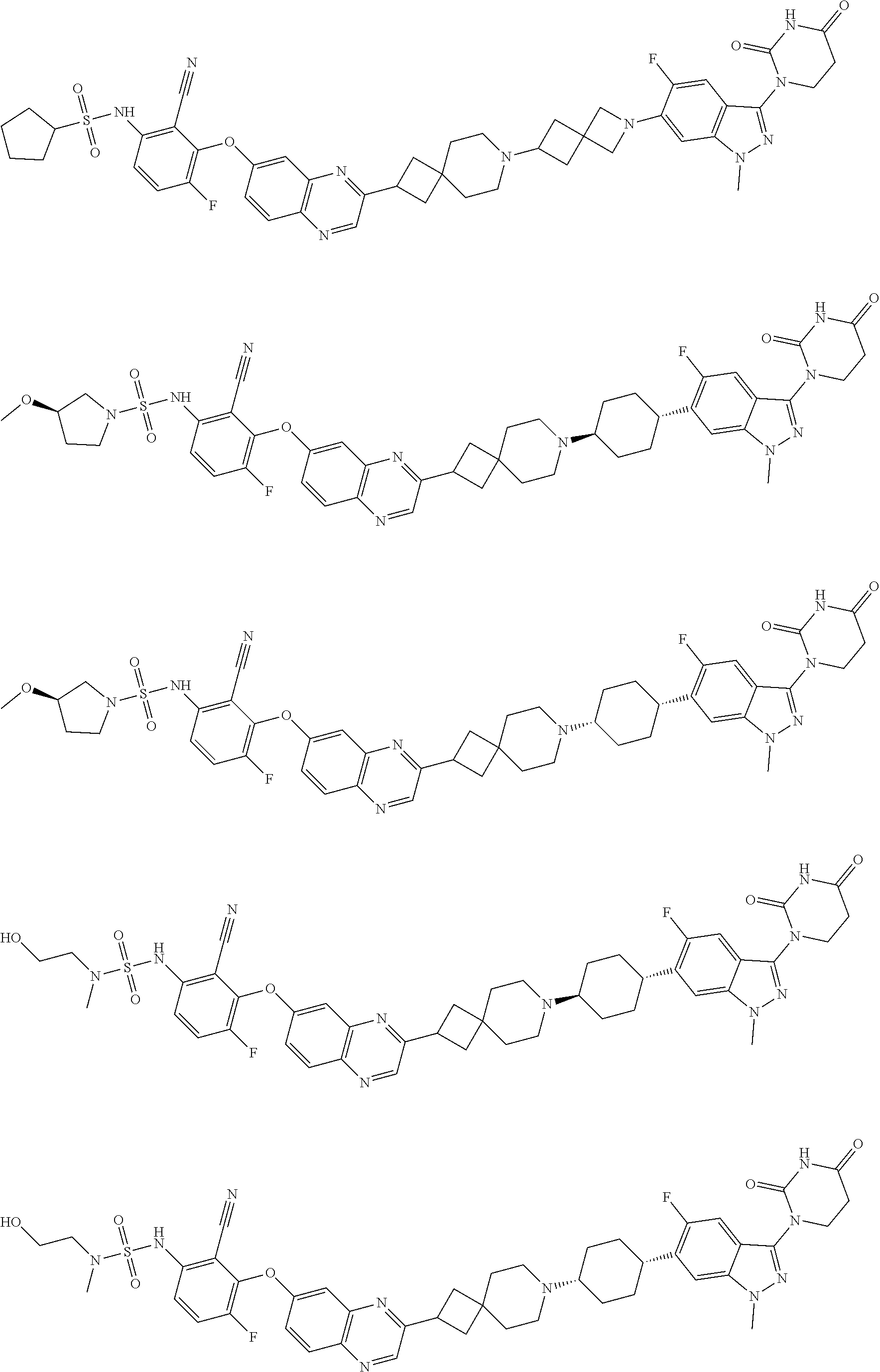

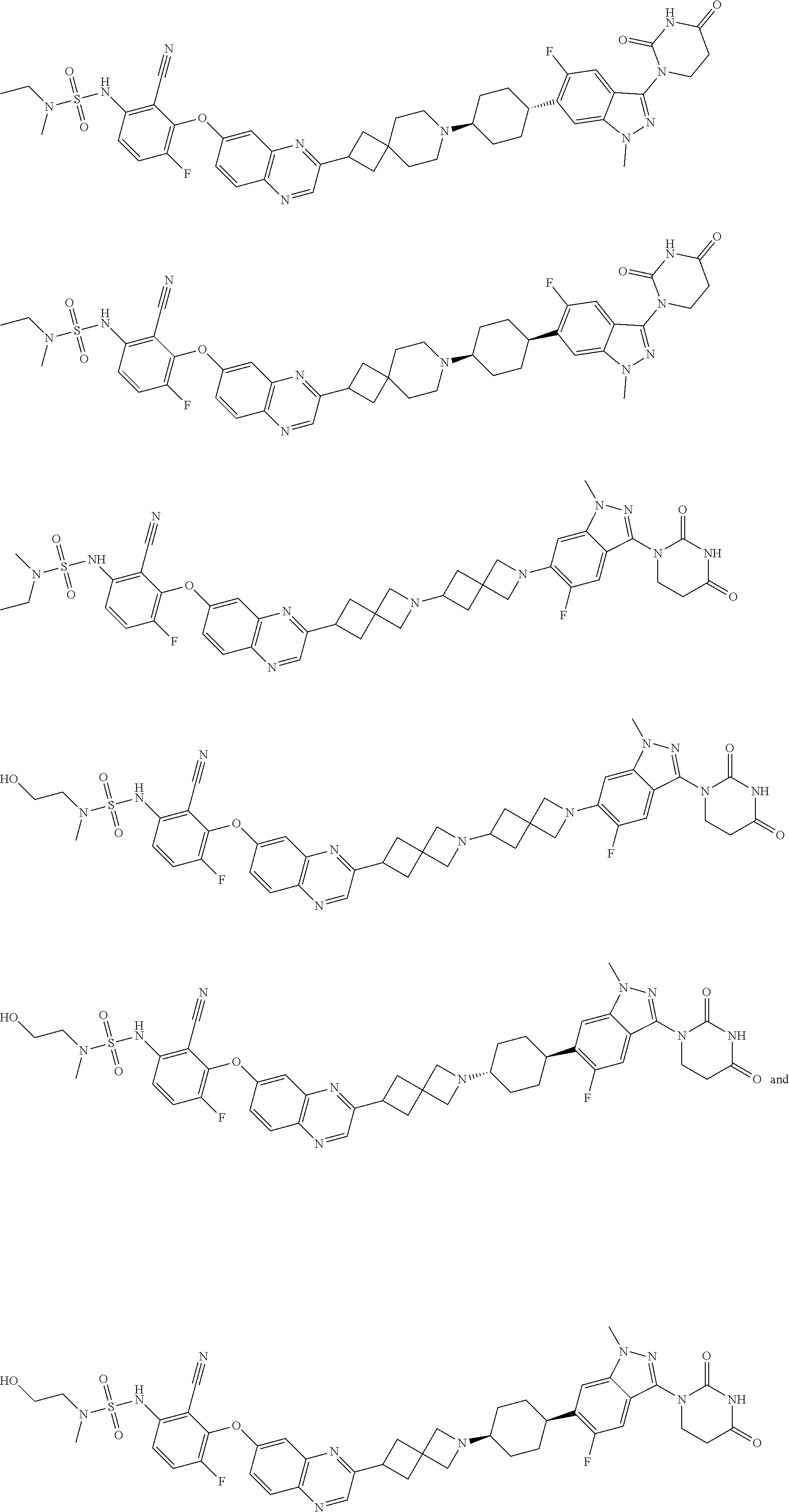

or a pharmaceutically acceptable salt thereof.

The invention further provides the following.

A compound of Formula VI or Formula XXII or a pharmaceutically acceptable salt thereof, for use as a therapeutically active substance in the treatment of a mutant BRAF mediated disorder.

A pharmaceutical composition comprising a compound of Formula VI or Formula XXII or a pharmaceutically acceptable salt thereof, and a pharmaceutically acceptable carrier.

The use of a compound of Formula VI or Formula XXII or a pharmaceutically acceptable salt thereof, for the therapeutic and/or prophylactic treatment of cancer.

A compound of Formula VI or Formula XXII or a pharmaceutically acceptable salt thereof, for use in the treatment and/or prophylaxis of cancer.

The use of a compound of Formula VI or Formula XXII or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula VI or Formula XXII or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula VII and Formula XXIII

In certain embodiments a compound of the invention is selected from the compound of Formula VII and the compound of Formula XXIII:

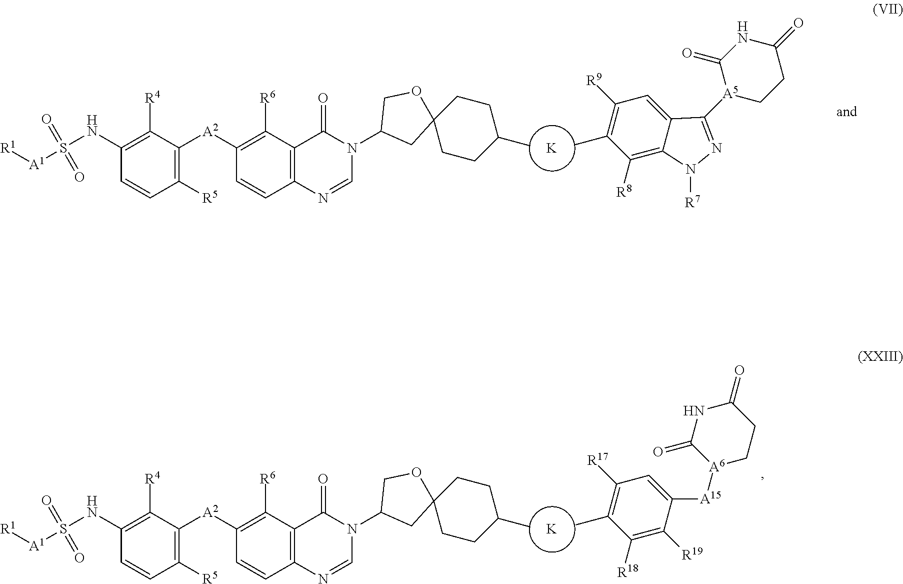

or pharmaceutically acceptable salt thereof,
wherein
Cycle \textcircled{K} is a cycloalkyl, 3,3-difluoro-piperidine-1,4-diyl, piperazine-1,4-diyl, bridged cycloalkyl, bridged heterocyclyl, or a spiroheterocyclyl containing 1 or 2 nitrogen atoms and 0 or 1 oxygen atom and containing no other heteroatoms, wherein the cycloalkyl piperazine-1,4-diyl, or a spiroheterocyclyl is optionally substituted with one or two substituents independently selected from halogen, alkyl and alkoxy; and all other substituents are as defined herein.

In certain embodiments the compound of Formula VII or Formula XXIII is selected from:

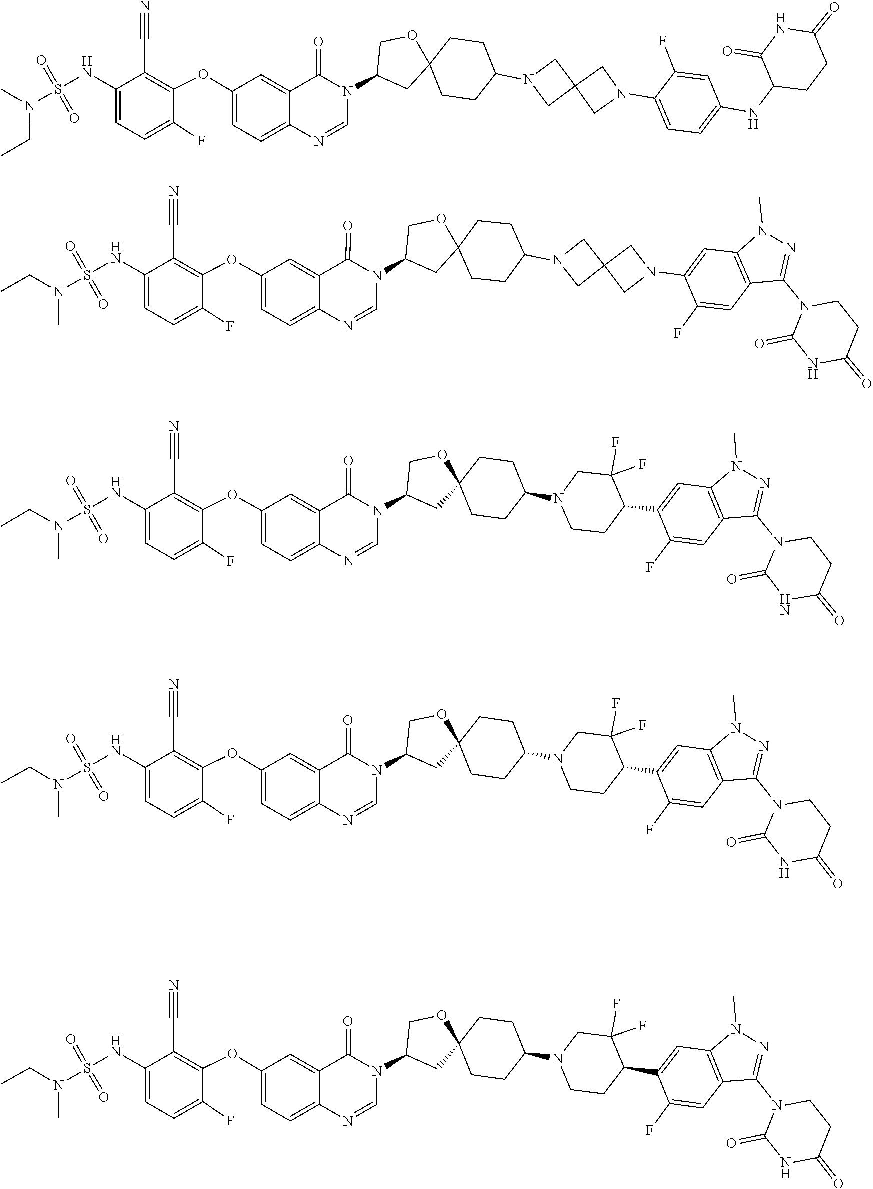

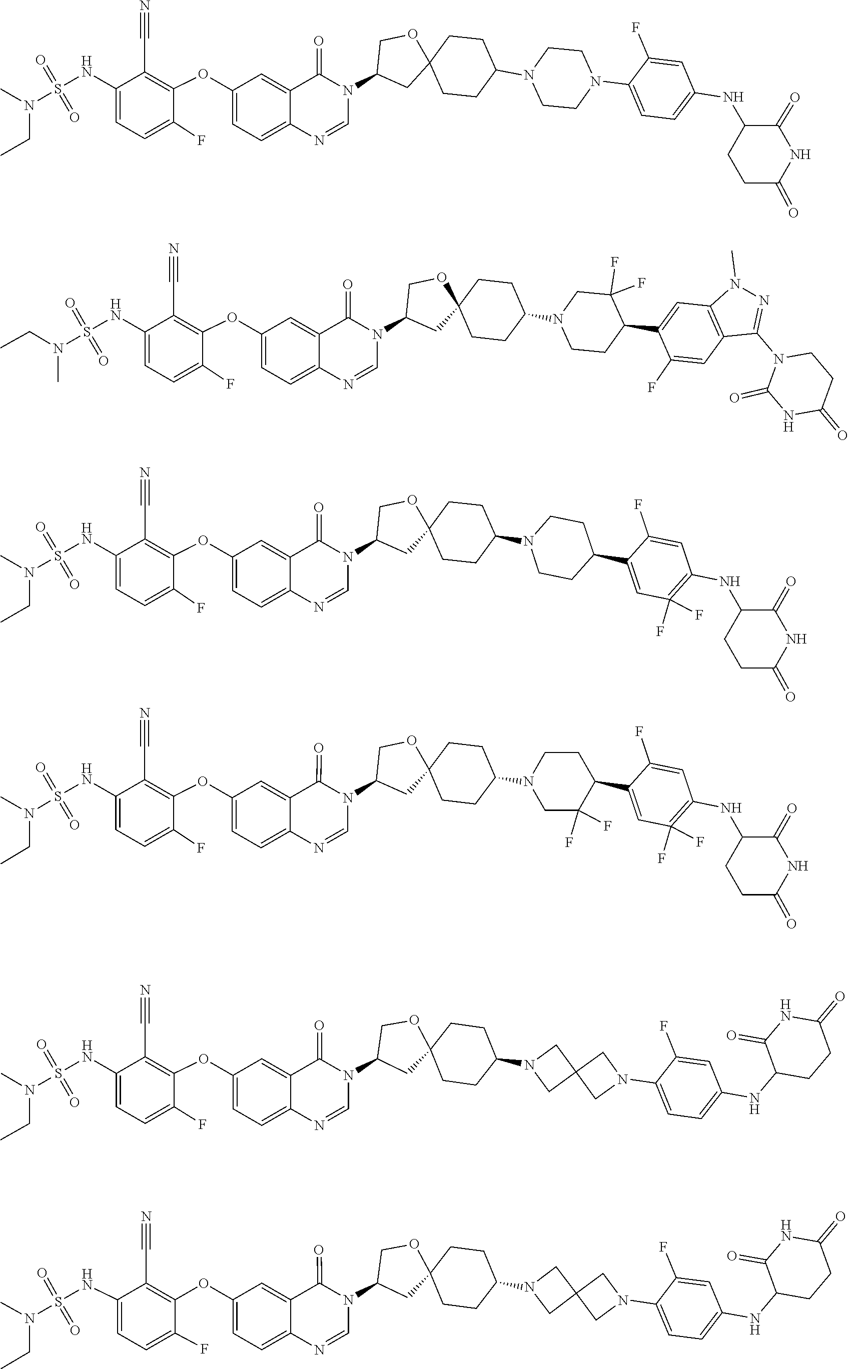

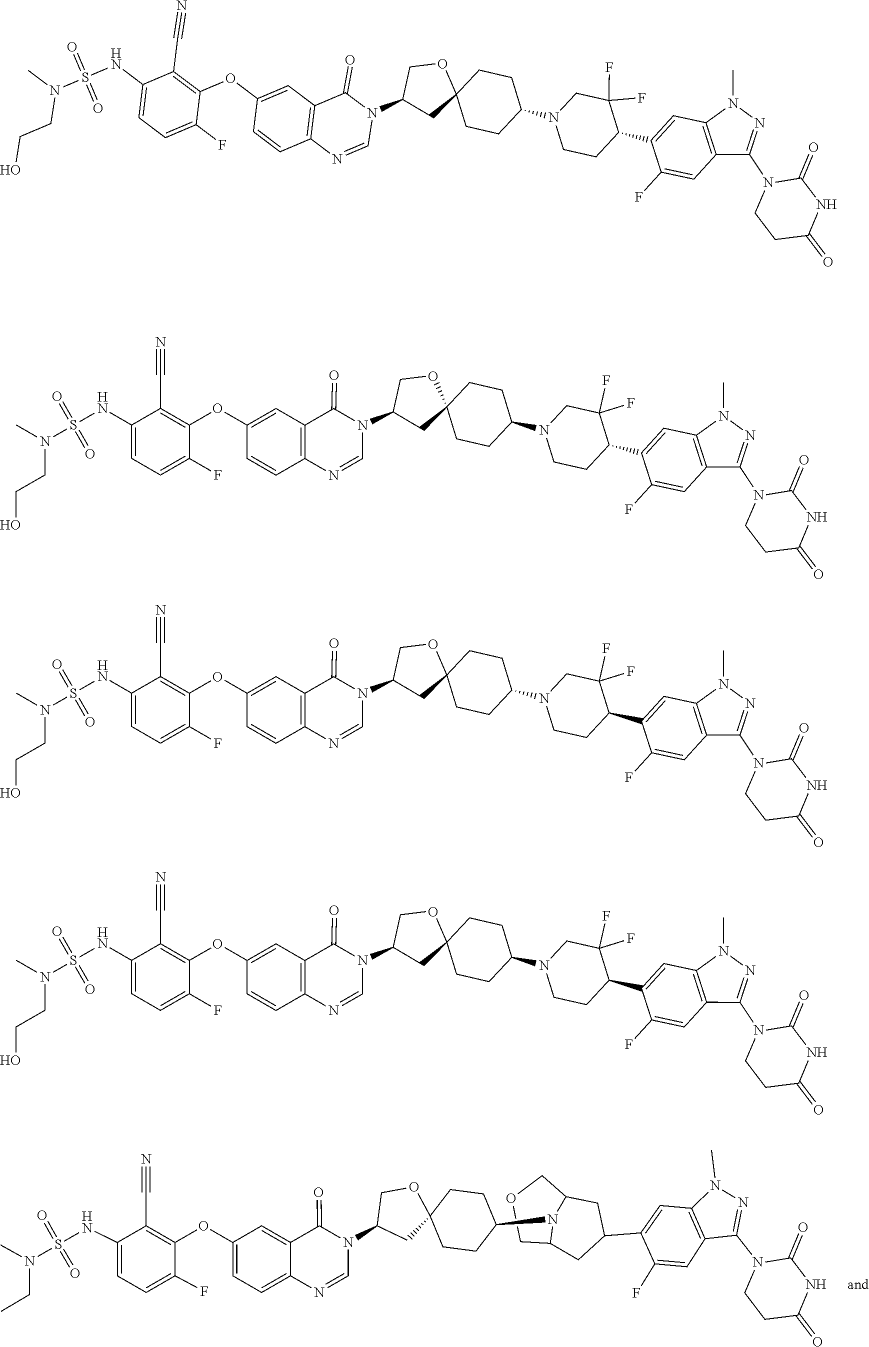

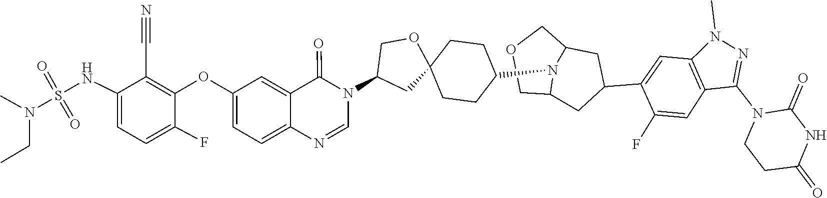

or pharmaceutically acceptable salt thereof.

The invention further provides the following.

A compound of Formula VII or Formula XXIII or a pharmaceutically acceptable salt thereof, for use as a therapeutically active substance in the treatment of a mutant BRAF mediated disorder.

A pharmaceutical composition comprising a compound of Formula VII or Formula XXIII or a pharmaceutically acceptable salt thereof, and a pharmaceutically acceptable carrier.

The use of a compound of Formula VII or Formula XXIII or a pharmaceutically acceptable salt thereof, for the therapeutic and/or prophylactic treatment of cancer.

A compound of Formula VII or Formula XXIII or a pharmaceutically acceptable salt thereof, for use in the treatment and/or prophylaxis of cancer.

The use of a compound of Formula VII or Formula XXIII or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula VII or Formula XXIII or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula VIII and Formula XXIV

In certain embodiments a compound of the invention is selected from the compound of Formula VIII and the compound of Formula XXIV:

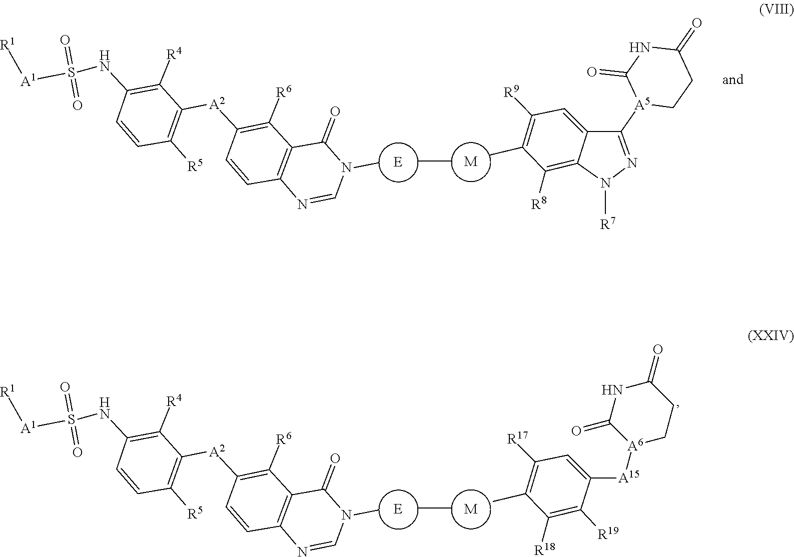

or pharmaceutically acceptable salt thereof,
wherein
Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
Cycle \textcircled{M} is a cycloalkyl substituted with 1, 2, or 3 substituents independently selected from the group consisting of alkyl, alkoxy, F, Cl, Br, and OH; and all other substituents are as defined herein.

In certain embodiments the compound of Formula VIII or Formula XXIV is selected from:

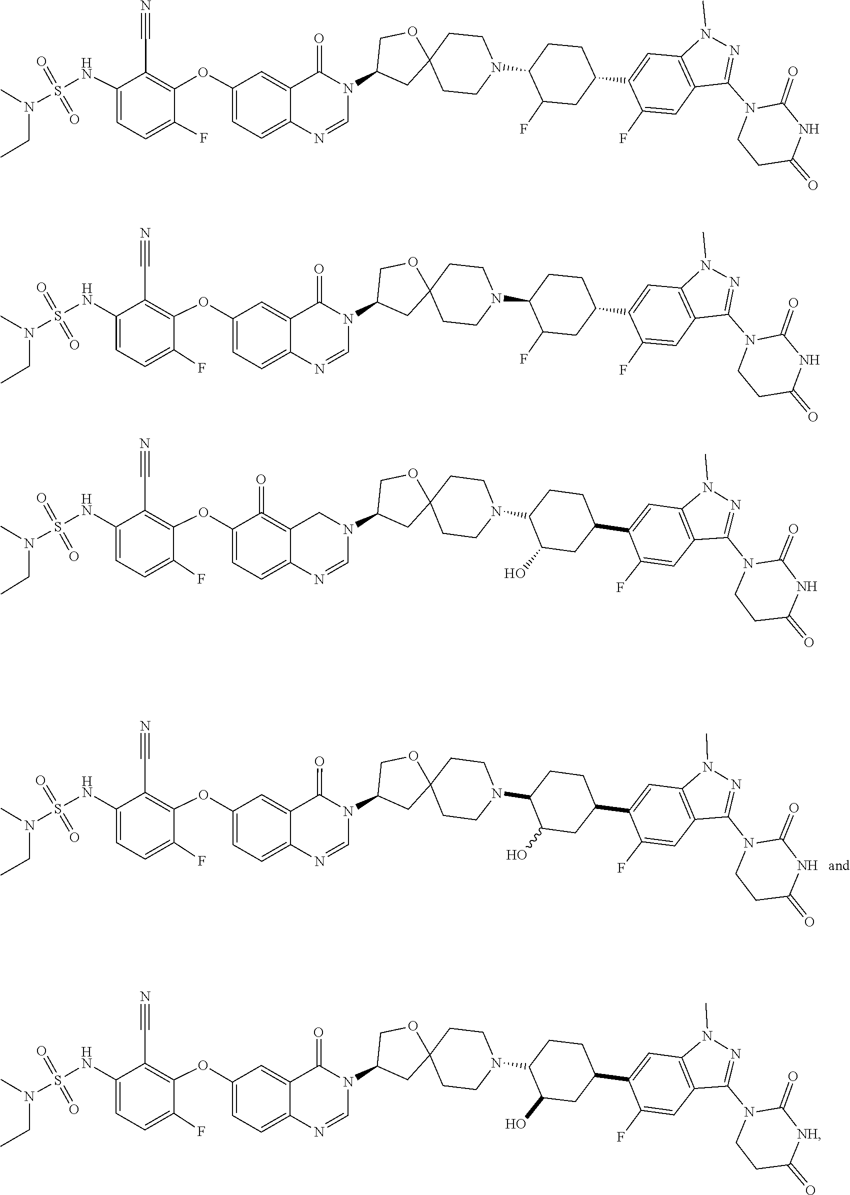

or pharmaceutically acceptable salt thereof.

The invention further provides the following.

A compound of Formula VIII or Formula XXIV or a pharmaceutically acceptable salt thereof, for use as a therapeutically active substance in the treatment of a mutant BRAF mediated disorder.

A pharmaceutical composition comprising a compound of Formula VIII or Formula XXIV or a pharmaceutically acceptable salt thereof, and a pharmaceutically acceptable carrier.

The use of a compound of Formula VIII or Formula XXIV or a pharmaceutically acceptable salt thereof, for the therapeutic and/or prophylactic treatment of cancer.

A compound of Formula VIII or Formula XXIV or a pharmaceutically acceptable salt thereof, for use in the treatment and/or prophylaxis of cancer.

The use of a compound of Formula VIII or Formula XXIV or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula VIII or Formula XXIV or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula IX and Formula XXV

In certain embodiments a compound of the invention is selected from the compound of Formula IX and the compound of Formula XXV:

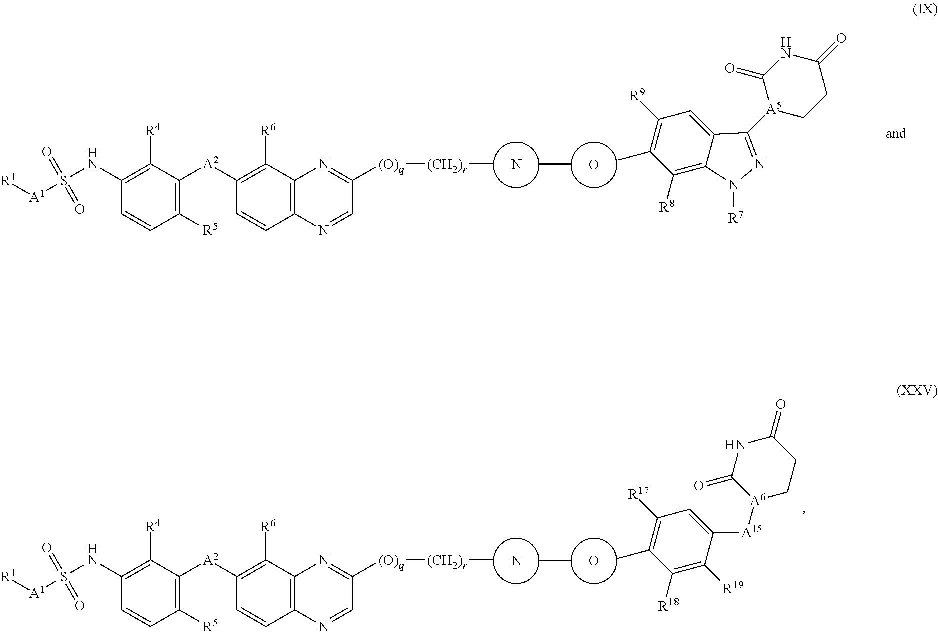

or pharmaceutically acceptable salt thereof,
wherein
Cycle \textcircled{N} is a cycloalkyl, spirocycloalkyl, heterocyclyl containing 1 or 2 nitrogen atoms and containing no other heteroatoms, wherein cycle \textcircled{N} is optionally substituted with one or two substituents independently selected from halogen, alkyl and alkoxy;
Cycle \textcircled{O} is a cycloalkyl, spirocycloalkyl, heterocyclyl containing 1 or 2 nitrogen atoms and containing no other heteroatoms, wherein cycle \textcircled{O} is optionally substituted with one or two substituents independently selected from halogen, alkyl and alkoxy; and all other substituents are as defined herein.

In certain embodiments the compound of Formula IX or Formula XXV is selected from:

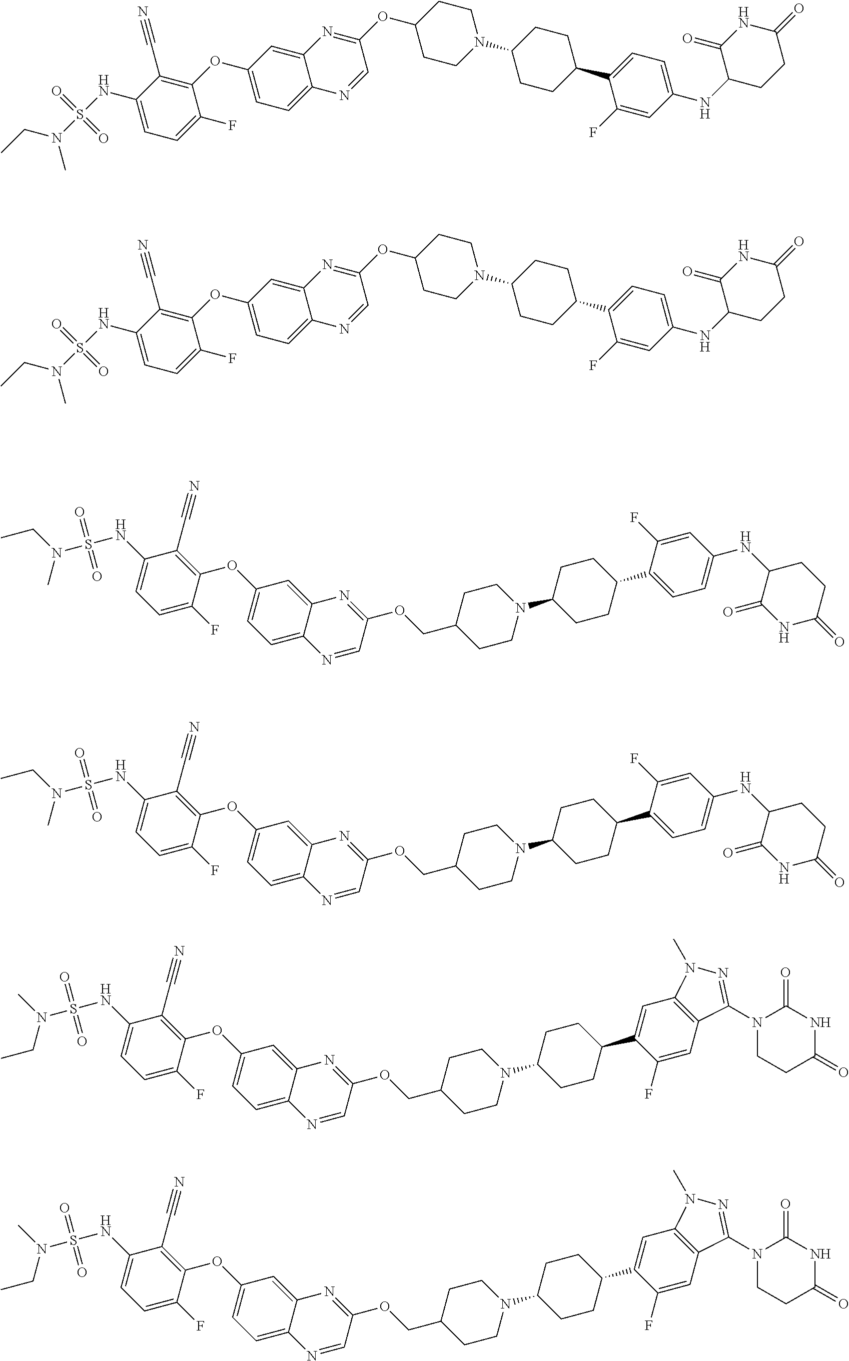

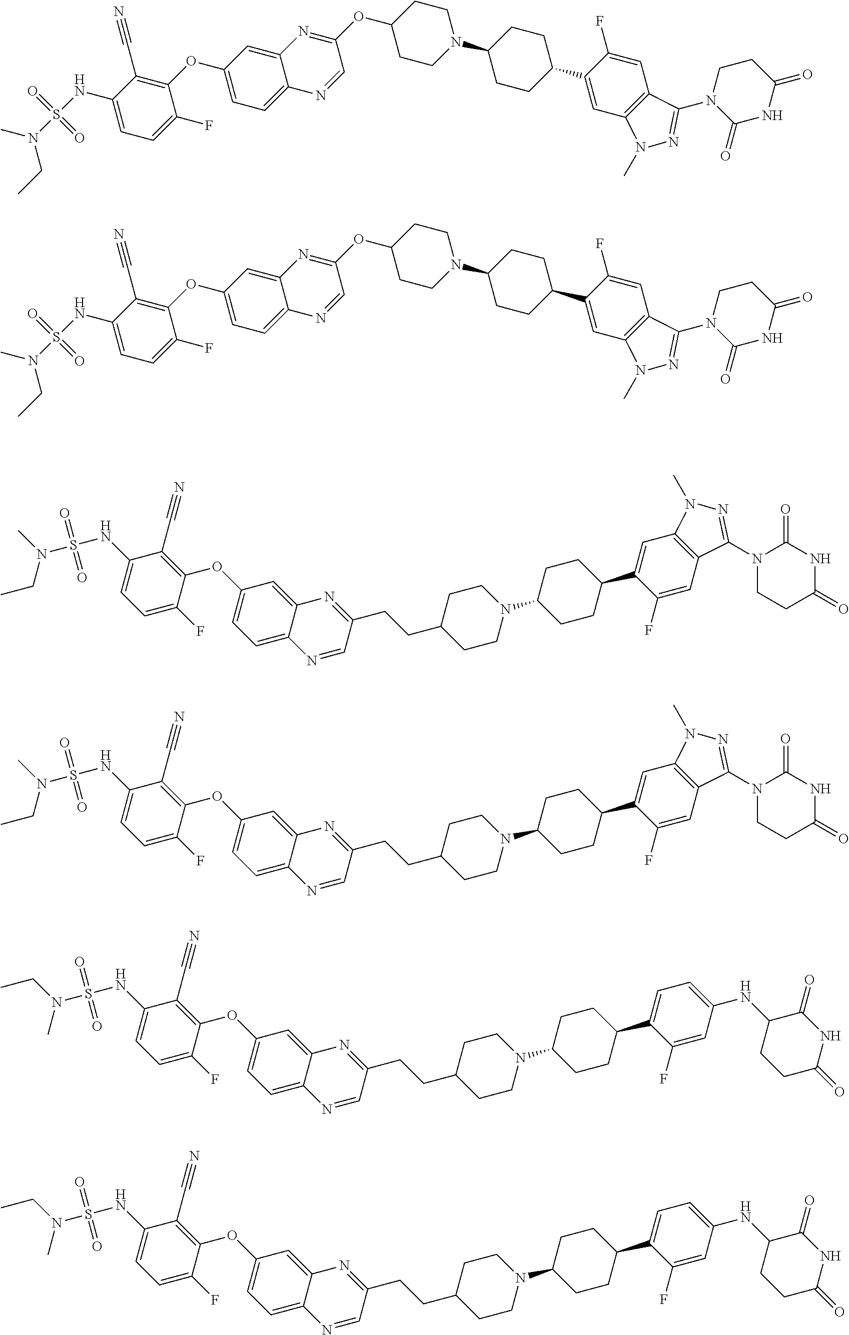

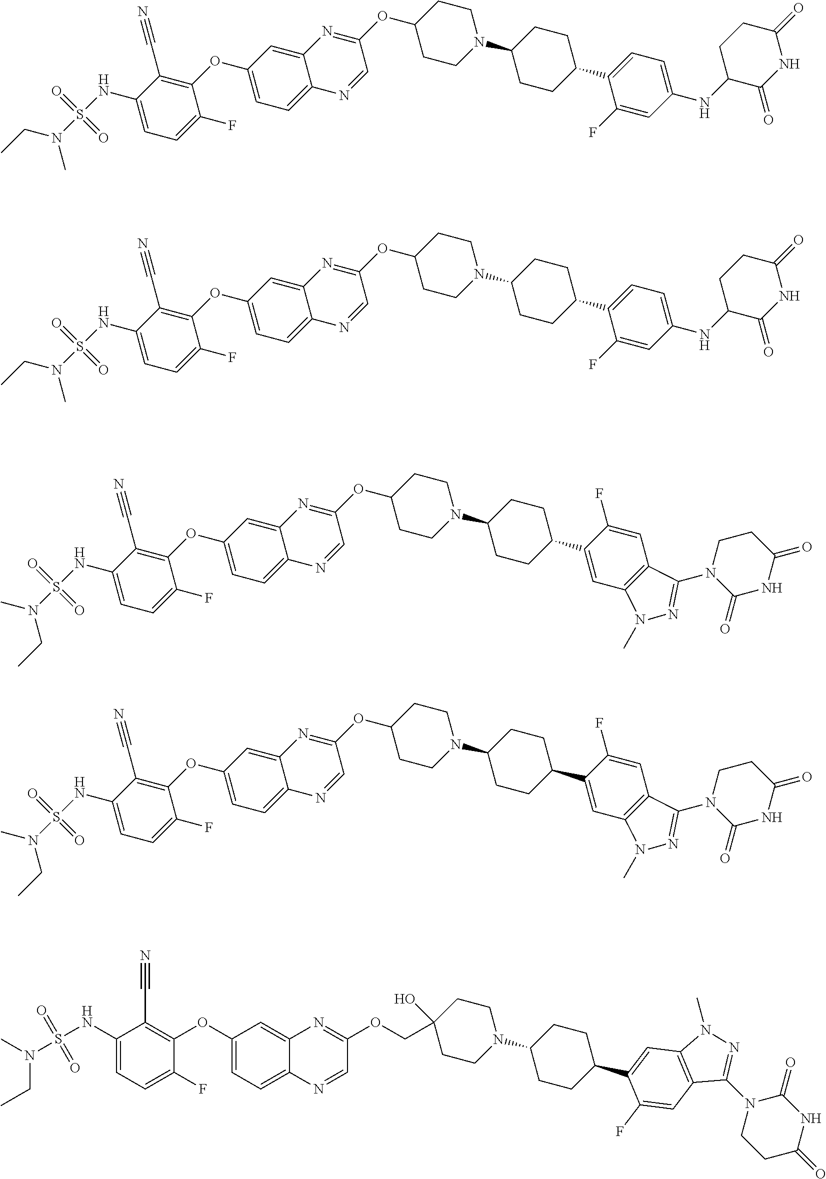

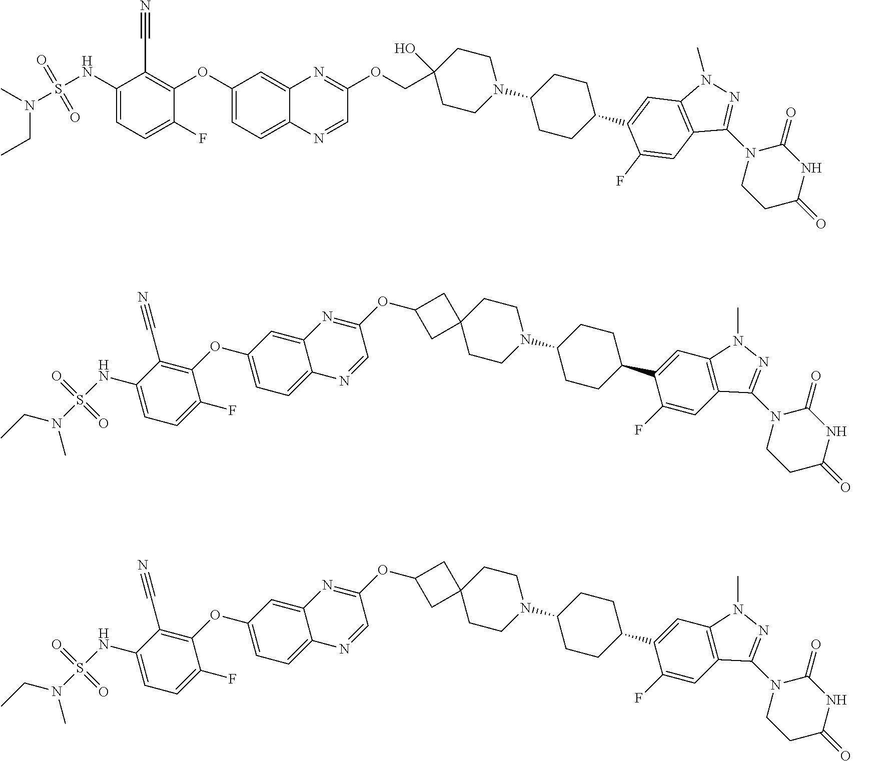

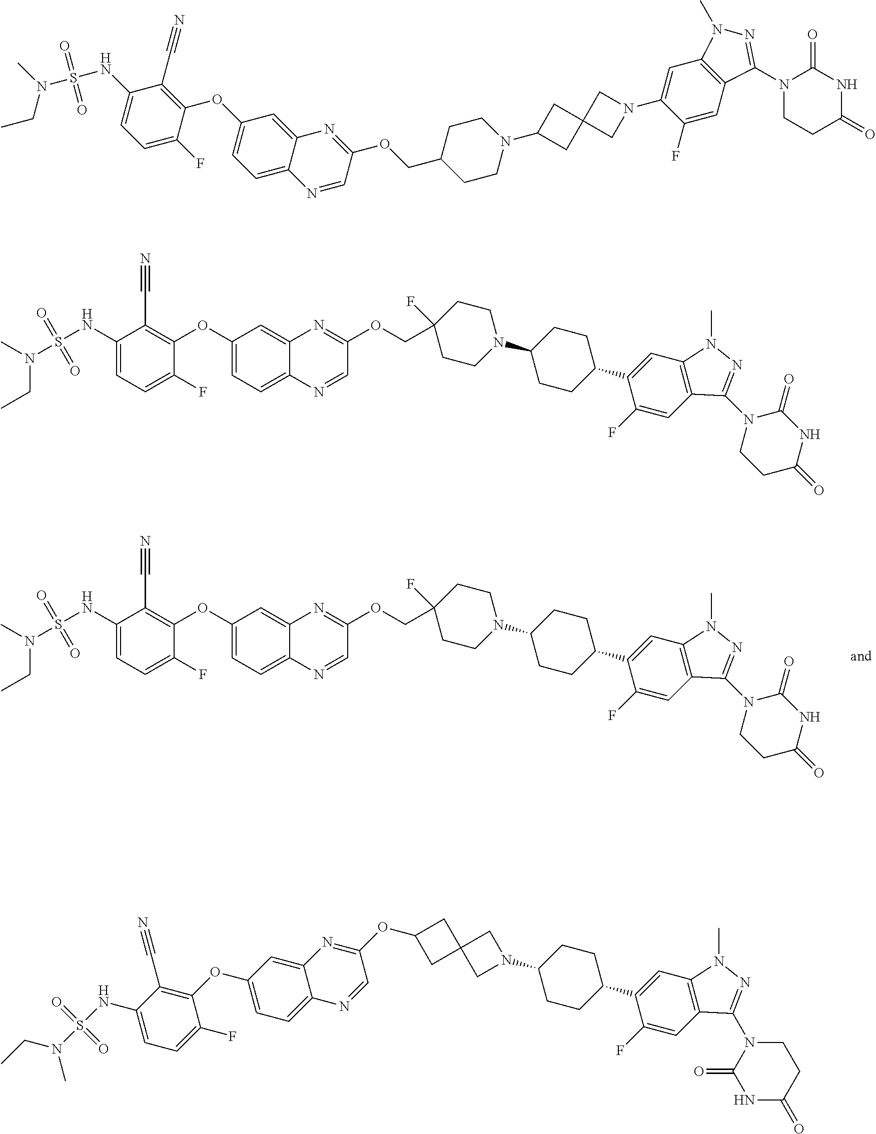 [SMILES: CCN(C)S(=O)(=O)Nc1ccc(F)c(Oc2ccc3ncc(OCC4CCN(C5CC6(C5)CN(c5cc7c(cc5F)c(N5CCC(=O)NC5=O)nn7C)C6)CC4)nc3c2)c1C#N ; CCN(C)S(=O)(=O)Nc1ccc(F)c(Oc2ccc3ncc(OCC4(F)CCN([C@H]5CC[C@H](c6cc7c(cc6F)c(N6CCC(=O)NC6=O)nn7C)CC5)CC4)nc3c2)c1C#N ; CCN(C)S(=O)(=O)Nc1ccc(F)c(Oc2ccc3ncc(OCC4(F)CCN([C@H]5CC[C@@H](c6cc7c(cc6F)c(N6CCC(=O)NC6=O)nn7C)CC5)CC4)nc3c2)c1C#N ; CCN(C)S(=O)(=O)Nc1ccc(F)c(Oc2ccc3ncc(OC4CC5(C4)CN([C@H]4CC[C@@H](c6cc7c(cc6F)c(N6CCC(=O)NC6=O)nn7C)CC4)C5)nc3c2)c1C#N]

or pharmaceutically acceptable salt thereof.

The invention further provides the following.

A compound of Formula IX or Formula XXV or a pharmaceutically acceptable salt thereof, for use as a therapeutically active substance in the treatment of a mutant BRAF mediated disorder.

A pharmaceutical composition comprising a compound of Formula IX or Formula XXV or a pharmaceutically acceptable salt thereof, and a pharmaceutically acceptable carrier.

The use of a compound of Formula IX or Formula XXV or a pharmaceutically acceptable salt thereof, for the therapeutic and/or prophylactic treatment of cancer.

A compound of Formula IX or Formula XXV or a pharmaceutically acceptable salt thereof, for use in the treatment and/or prophylaxis of cancer.

The use of a compound of Formula IX or Formula XXV or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula IX or Formula XXV or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula X and Formula XXVI

In certain embodiments a compound of the invention is selected from the compound of Formula X and the compound of Formula XXVI:

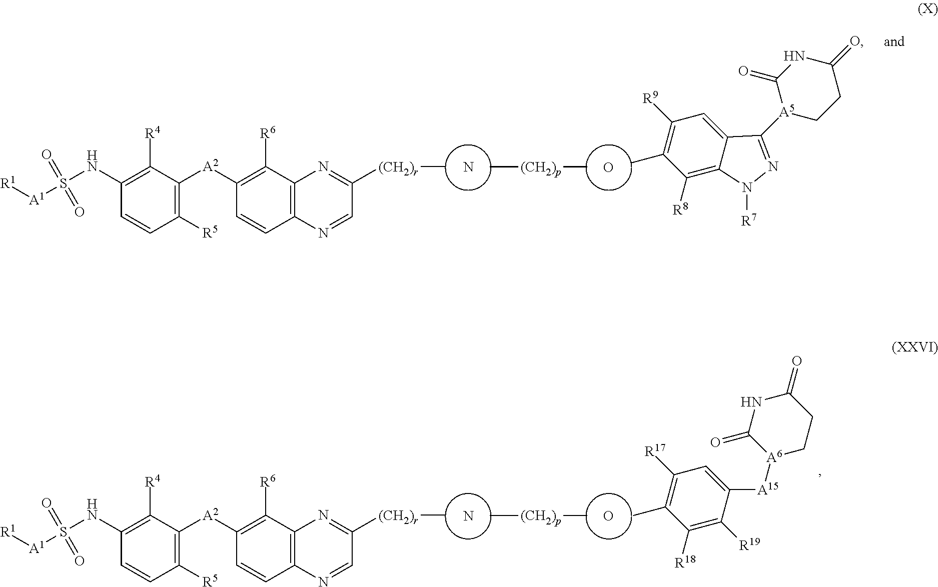

or a pharmaceutically acceptable salt thereof,
wherein:
Cycle \textcircled{N} is a cycloalkyl, spirocycloalkyl, heterocyclyl containing 1 or 2 nitrogen atoms and containing no other heteroatoms, wherein cycle \textcircled{N} is optionally substituted with one or two substituents independently selected from halogen, alkyl and alkoxy;
Cycle \textcircled{O} is a cycloalkyl, spirocycloalkyl, heterocyclyl containing 1 or 2 nitrogen atoms and containing no other heteroatoms, wherein cycle \textcircled{O} is optionally substituted with one or two substituents independently selected from halogen, alkyl and alkoxy; and all other substituents are as defined herein.

In certain embodiments the compound of Formula X or Formula XXVI is selected from:

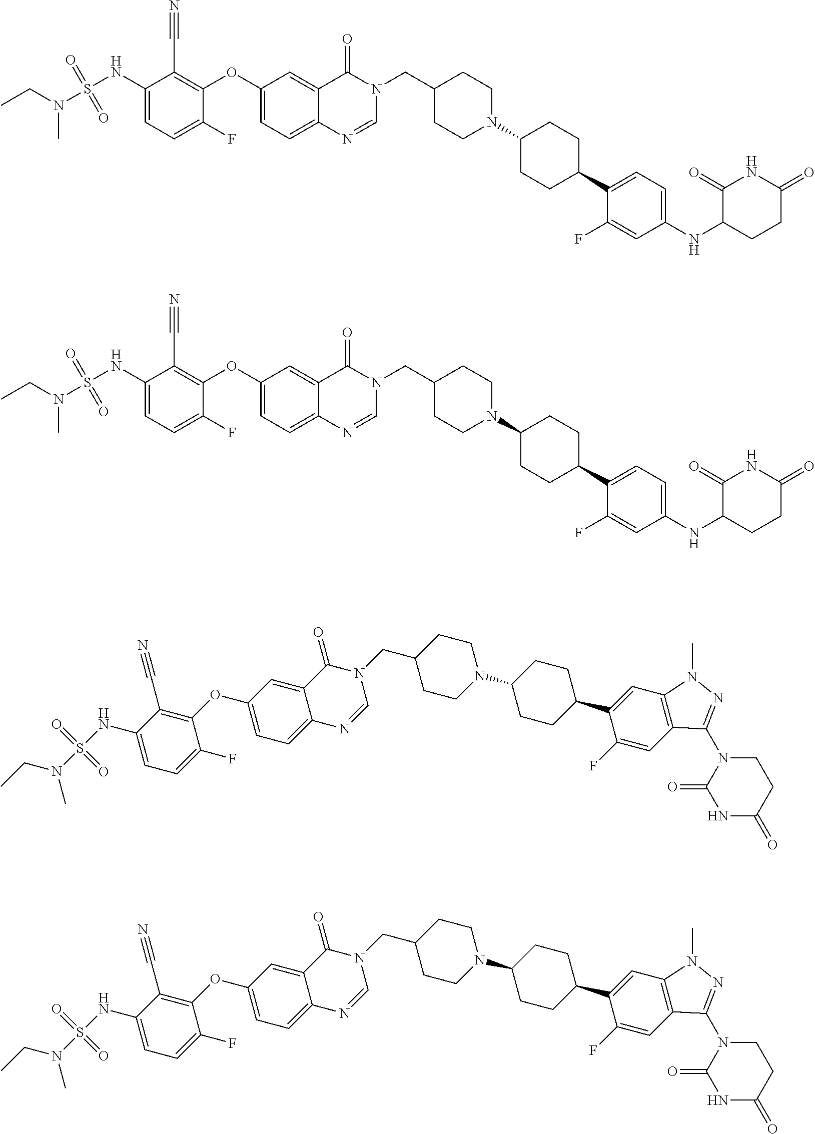 [SMILES: CCN(C)S(=O)(=O)Nc1ccc(F)c(Oc2ccc3ncn(CC4CCN([C@H]5CC[C@H](c6ccc(NC7CCC(=O)NC7=O)cc6F)CC5)CC4)c(=O)c3c2)c1C#N ; CCN(C)S(=O)(=O)Nc1ccc(F)c(Oc2ccc3ncn(CC4CCN([C@H]5CC[C@@H](c6ccc(NC7CCC(=O)NC7=O)cc6F)CC5)CC4)c(=O)c3c2)c1C#N ; CCN(C)S(=O)(=O)Nc1ccc(F)c(Oc2ccc3ncn(CC4CCN([C@H]5CC[C@H](c6cc7c(cc6F)c(N6CCC(=O)NC6=O)nn7C)CC5)CC4)c(=O)c3c2)c1C#N ; CCN(C)S(=O)(=O)Nc1ccc(F)c(Oc2ccc3ncn(CC4CCN([C@H]5CC[C@@H](c6cc7c(cc6F)c(N6CCC(=O)NC6=O)nn7C)CC5)CC4)c(=O)c3c2)c1C#N]

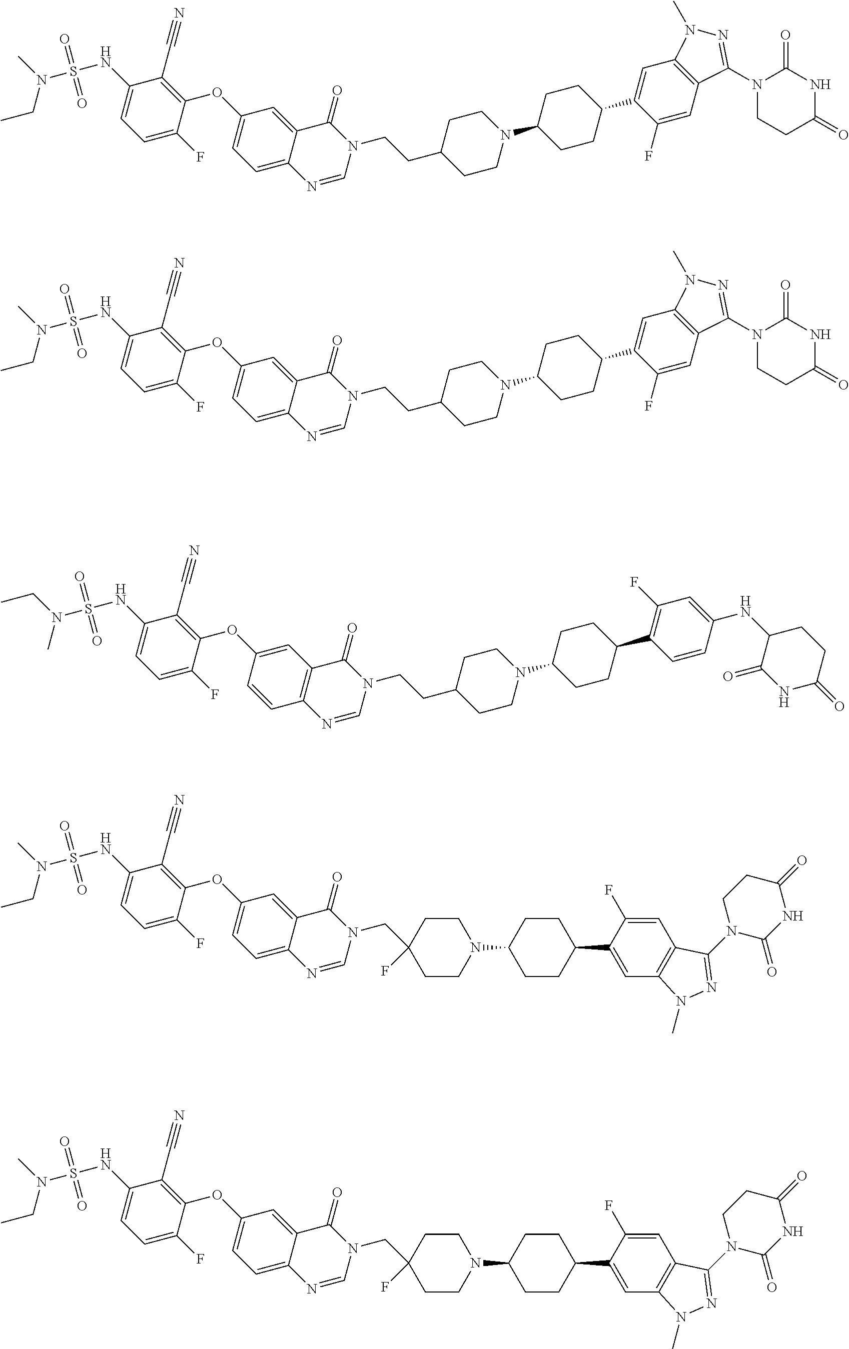

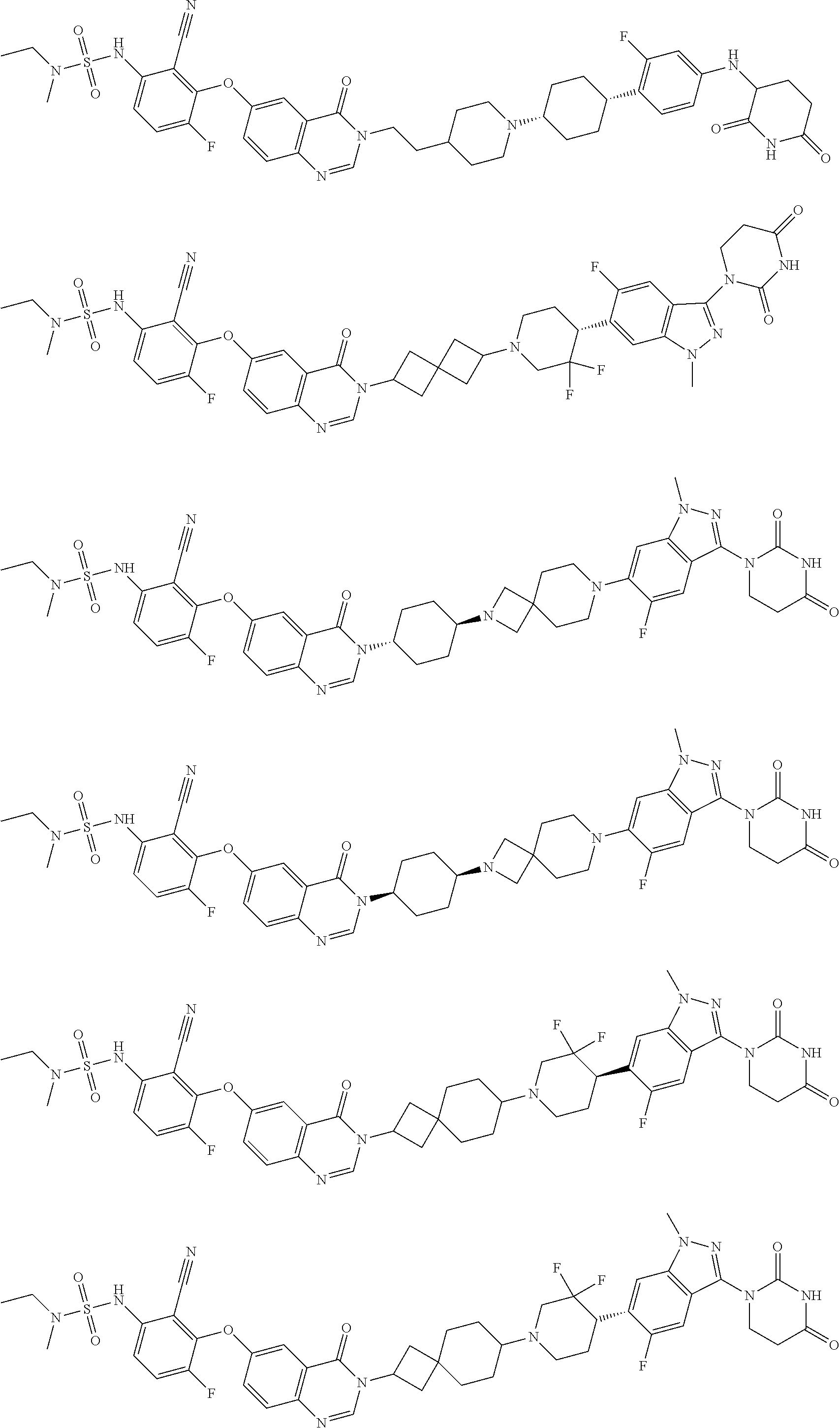

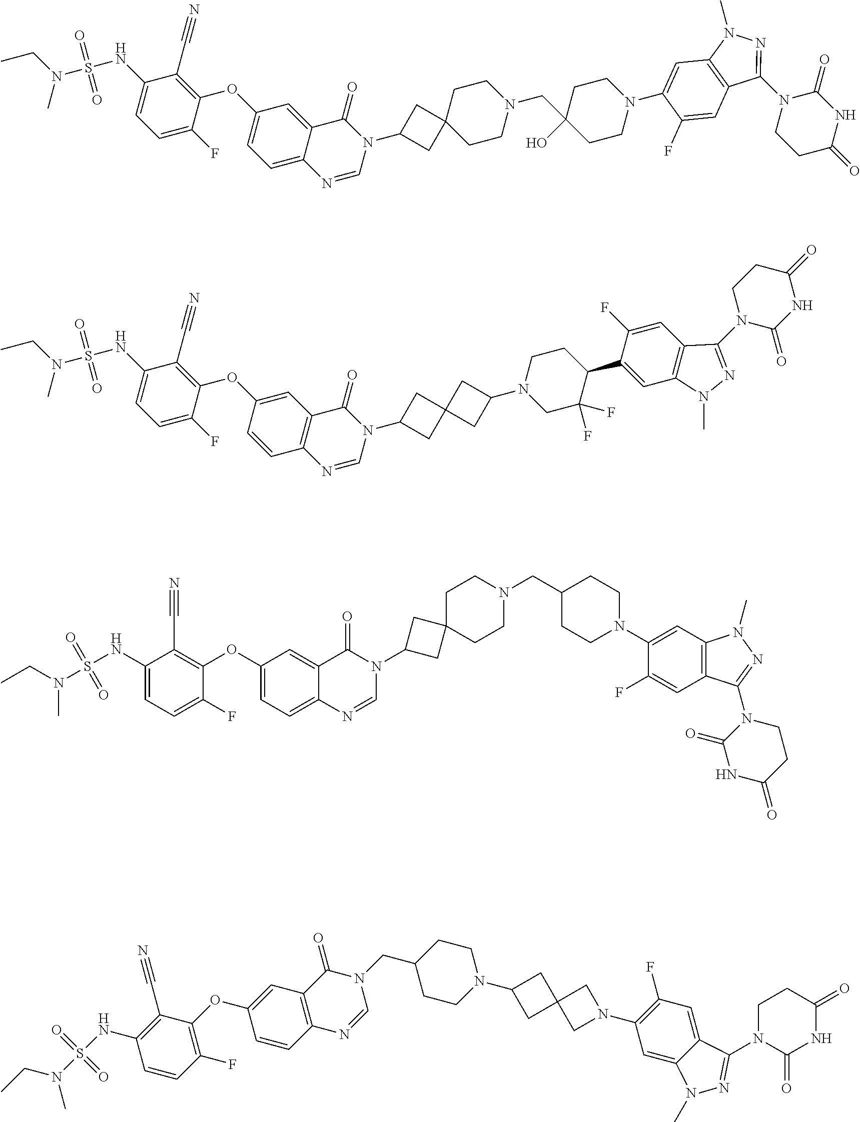 [SMILES: CCN(C)S(=O)(=O)Nc1ccc(F)c(Oc2ccc3ncn(C4CC5(CCN(CC6(O)CCN(c7cc8c(cc7F)c(N7CCC(=O)NC7=O)nn8C)CC6)CC5)C4)c(=O)c3c2)c1C#N ; CCN(C)S(=O)(=O)Nc1ccc(F)c(Oc2ccc3ncn(C4CC5(CC(N6CC[C@@H](c7cc8c(cc7F)c(N7CCC(=O)NC7=O)nn8C)C(F)(F)C6)C5)C4)c(=O)c3c2)c1C#N ; CCN(C)S(=O)(=O)Nc1ccc(F)c(Oc2ccc3ncn(C4CC5(CCN(CC6CCN(c7cc8c(cc7F)c(N7CCC(=O)NC7=O)nn8C)CC6)CC5)C4)c(=O)c3c2)c1C#N ; CCN(C)S(=O)(=O)Nc1ccc(F)c(Oc2ccc3ncn(CC4CCN(C5CC6(C5)CN(c5cc7c(cc5F)c(N5CCC(=O)NC5=O)nn7C)C6)CC4)c(=O)c3c2)c1C#N]

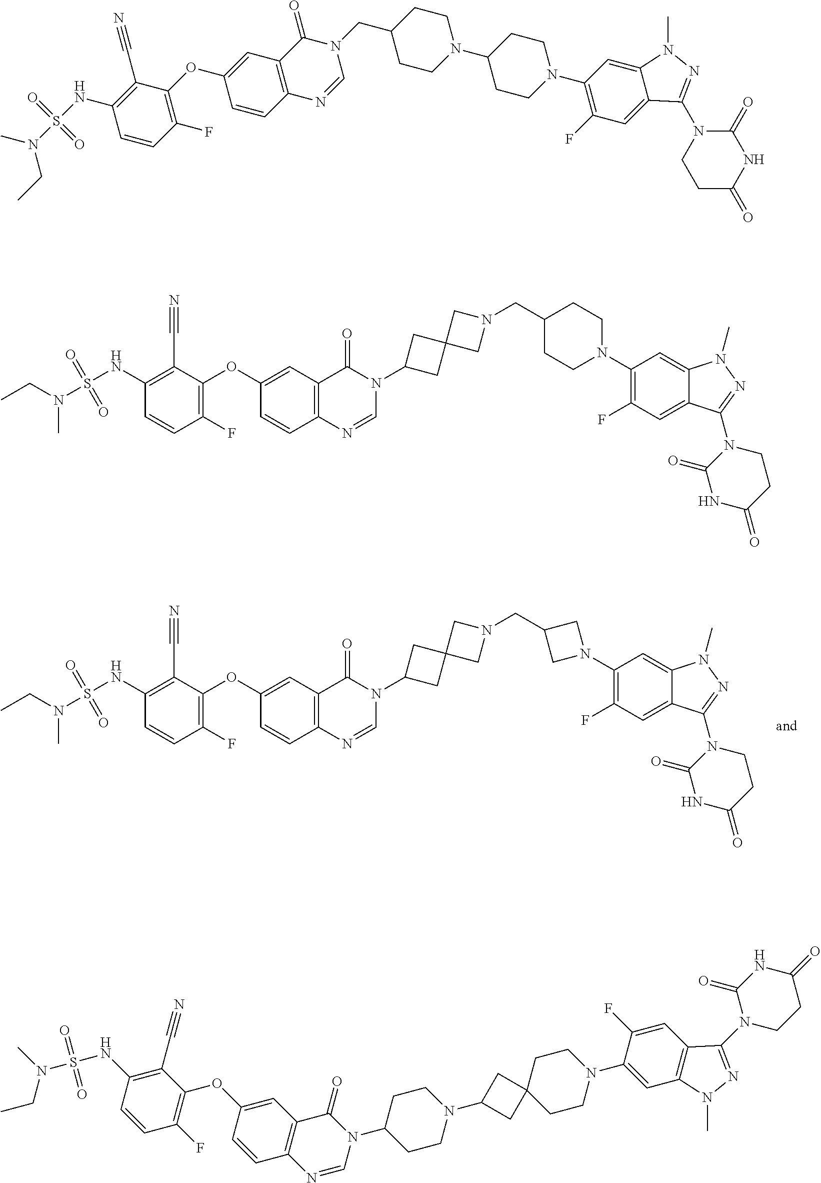 [SMILES: CCN(C)S(=O)(=O)Nc1ccc(F)c(Oc2ccc3ncn(CC4CCN(C5CCN(c6cc7c(cc6F)c(N6CCC(=O)NC6=O)nn7C)CC5)CC4)c(=O)c3c2)c1C#N ; CCN(C)S(=O)(=O)Nc1ccc(F)c(Oc2ccc3ncn(C4CC5(C4)CN(CC4CCN(c6cc7c(cc6F)c(N6CCC(=O)NC6=O)nn7C)CC4)C5)c(=O)c3c2)c1C#N ; CCN(C)S(=O)(=O)Nc1ccc(F)c(Oc2ccc3ncn(C4CC5(C4)CN(CC4CN(c6cc7c(cc6F)c(N6CCC(=O)NC6=O)nn7C)C4)C5)c(=O)c3c2)c1C#N ; CCN(C)S(=O)(=O)Nc1ccc(F)c(Oc2ccc3ncn(C4CCN(C5CC6(CCN(c7cc8c(cc7F)c(N7CCC(=O)NC7=O)nn8C)CC6)C5)CC4)c(=O)c3c2)c1C#N]

or pharmaceutically acceptable salt thereof.

The invention further provides the following.

A compound of Formula X or Formula XXVI or a pharmaceutically acceptable salt thereof, for use as a therapeutically active substance in the treatment of a mutant BRAF mediated disorder.

A pharmaceutical composition comprising a compound of Formula X or Formula XXVI or a pharmaceutically acceptable salt thereof, and a pharmaceutically acceptable carrier.

The use of a compound of Formula X or Formula XXVI or a pharmaceutically acceptable salt thereof, for the therapeutic and/or prophylactic treatment of cancer.

A compound of Formula X or Formula XXVI or a pharmaceutically acceptable salt thereof, for use in the treatment and/or prophylaxis of cancer.

The use of a compound of Formula X or Formula XXVI or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula X or Formula XXVI or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula XI and Formula XXVII

In certain embodiments a compound of the invention is selected from the compound of Formula XI and the compound of Formula XXVII:

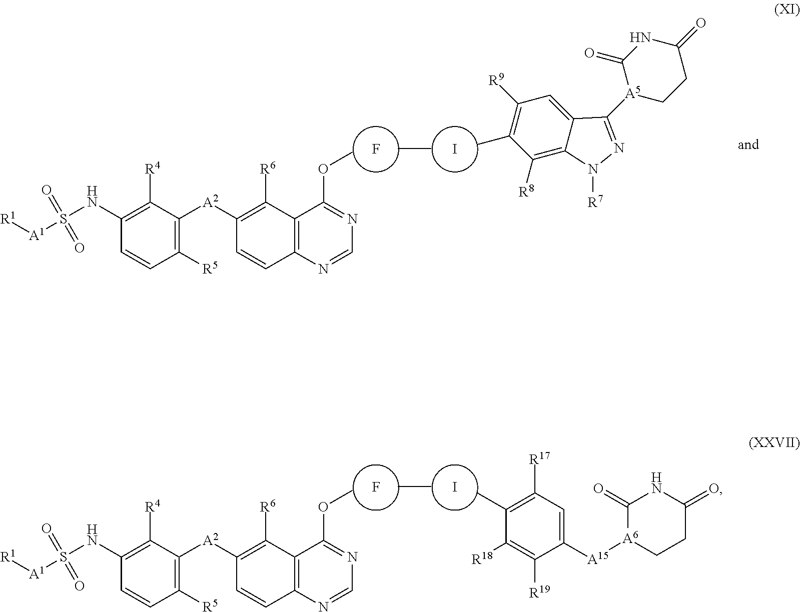

or a pharmaceutically acceptable salt h f (XXVII),
wherein
Cycle \textcircled{F} is a bridged heterocyclyl or spiroheterocyclyl containing 1 or 2 nitrogen atoms and containing no other heteroatoms, wherein cycle \textcircled{F} is optionally substituted with one or two substituents independently selected from halogen, alkyl and alkoxy;
Cycle \textcircled{I} is a cycloalkyl, wherein cycle \textcircled{I} is optionally substituted with one or two substituents independently selected from halogen, alkyl and alkoxy; and all other substituents are as defined herein.

In certain embodiments the compound of Formula XI or Formula XXVII is selected from:

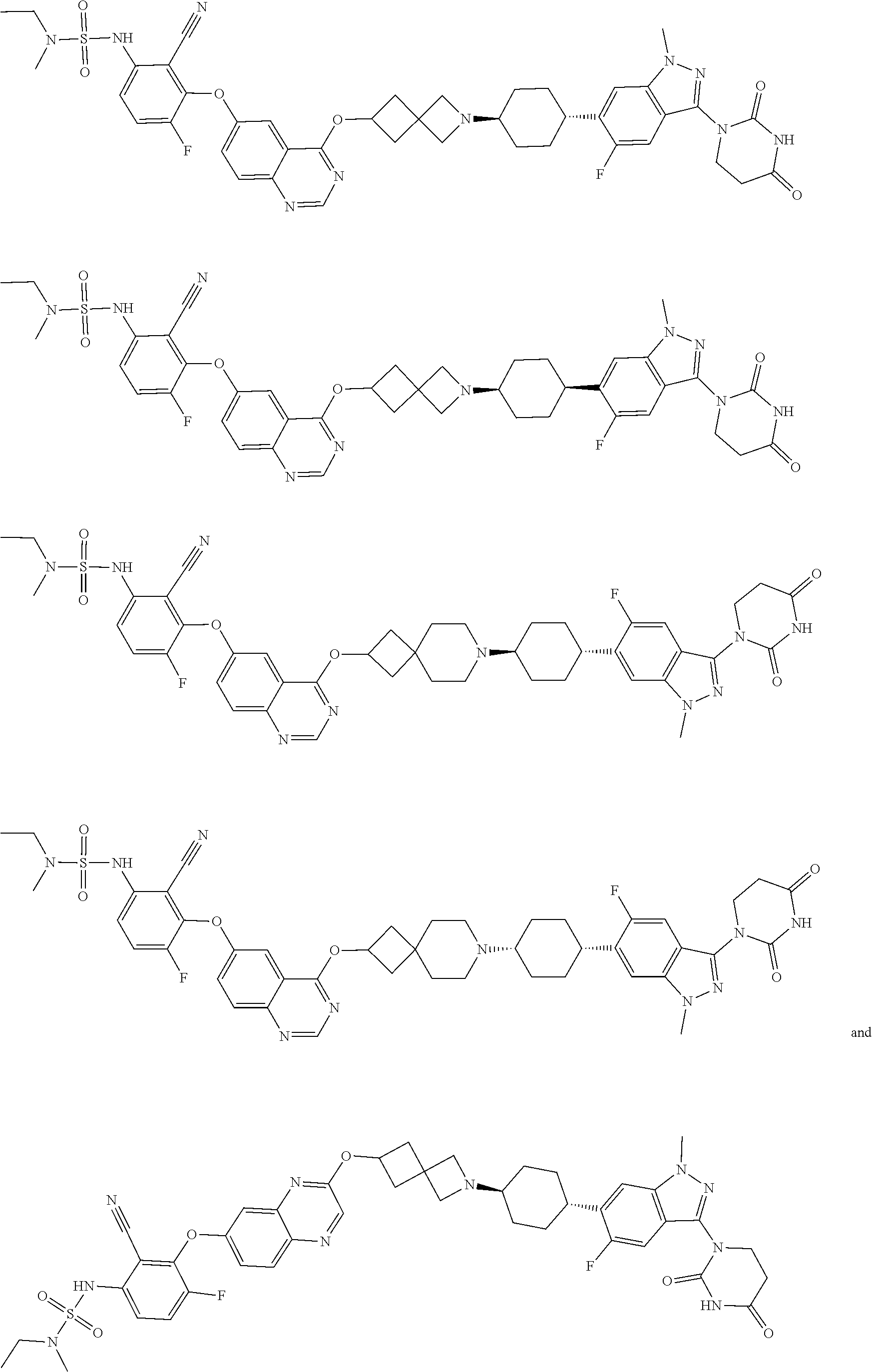

or pharmaceutically acceptable salt thereof.

The invention further provides the following.

A compound of Formula XI or Formula XXVII or a pharmaceutically acceptable salt thereof, for use as a therapeutically active substance in the treatment of a mutant BRAF mediated disorder.

A pharmaceutical composition comprising a compound of Formula XI or Formula XXVII or a pharmaceutically acceptable salt thereof, and a pharmaceutically acceptable carrier.

The use of a compound of Formula XI or Formula XXVII or a pharmaceutically acceptable salt thereof, for the therapeutic and/or prophylactic treatment of cancer.

A compound of Formula XI or Formula XXVII or a pharmaceutically acceptable salt thereof, for use in the treatment and/or prophylaxis of cancer.

The use of a compound of Formula XI or Formula XXVII or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula XI or Formula XXVII or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula XII and Formula XXVIII

In certain embodiments a compound of the invention is selected from the compound of Formula XII and the compound of Formula XXVIII:

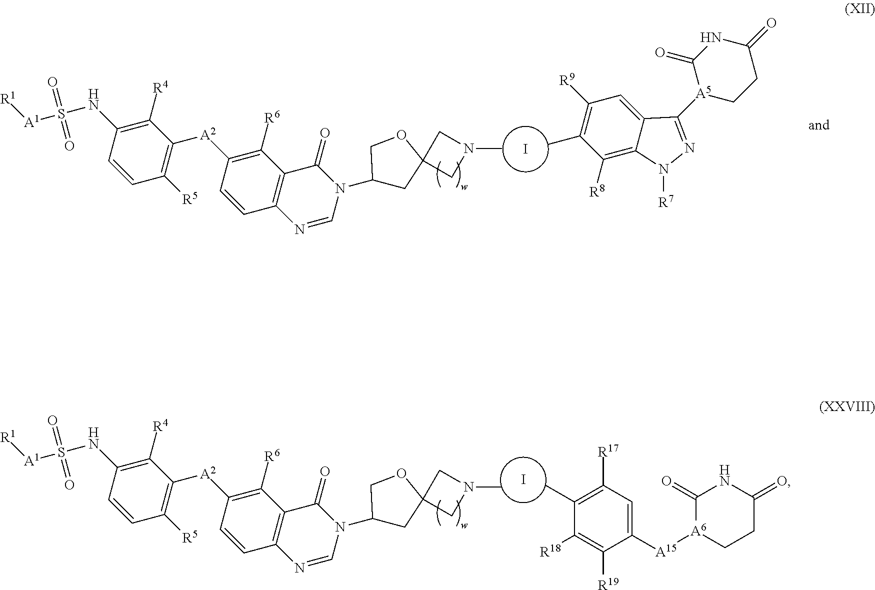

or a pharmaceutically acceptable salt thereof,
wherein
Cycle \textcircled{I} is a cycloalkyl, wherein cycle \textcircled{I} is optionally substituted with one or two substituents independently selected from halogen, alkyl and alkoxy; and all other substituents are as defined herein.

In certain embodiments the compound of Formula XII or Formula XXVIII is selected from:

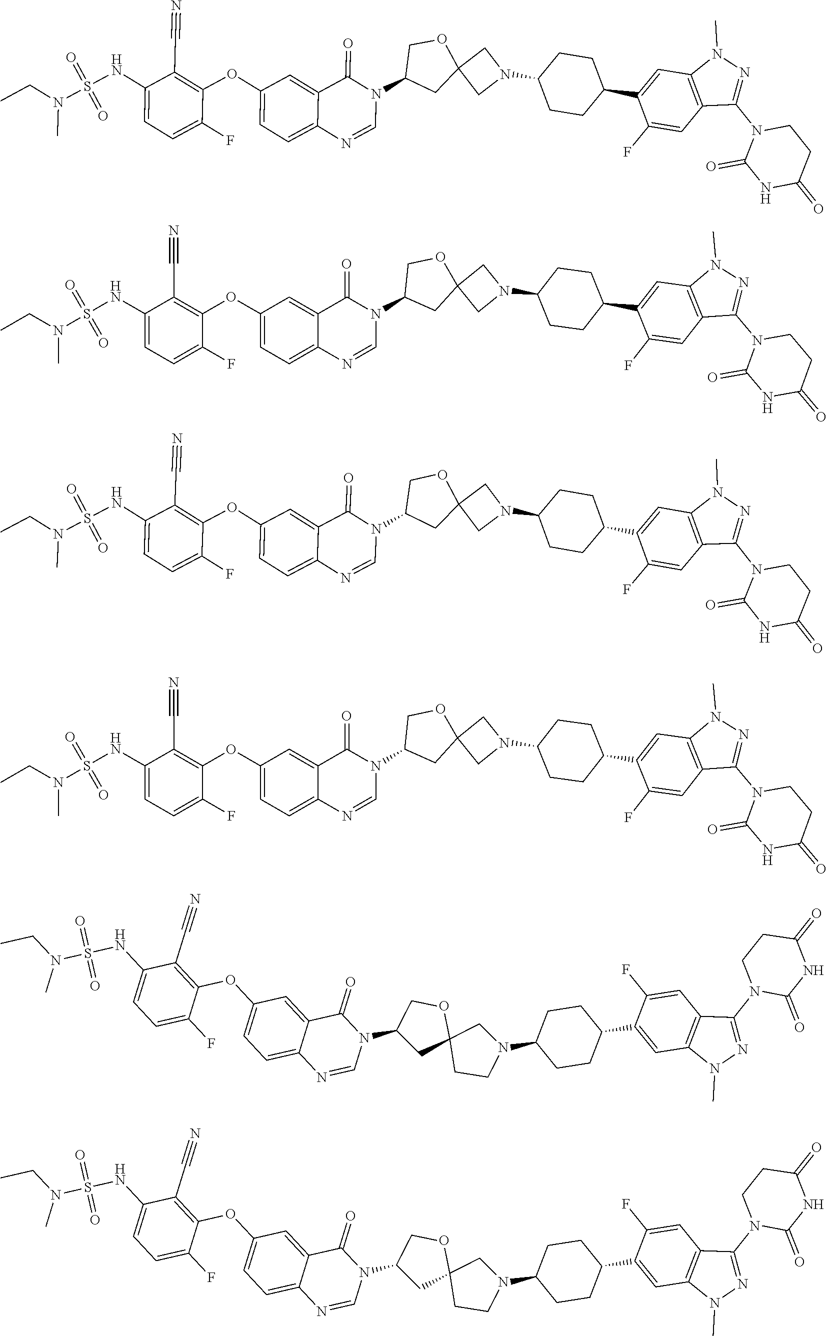

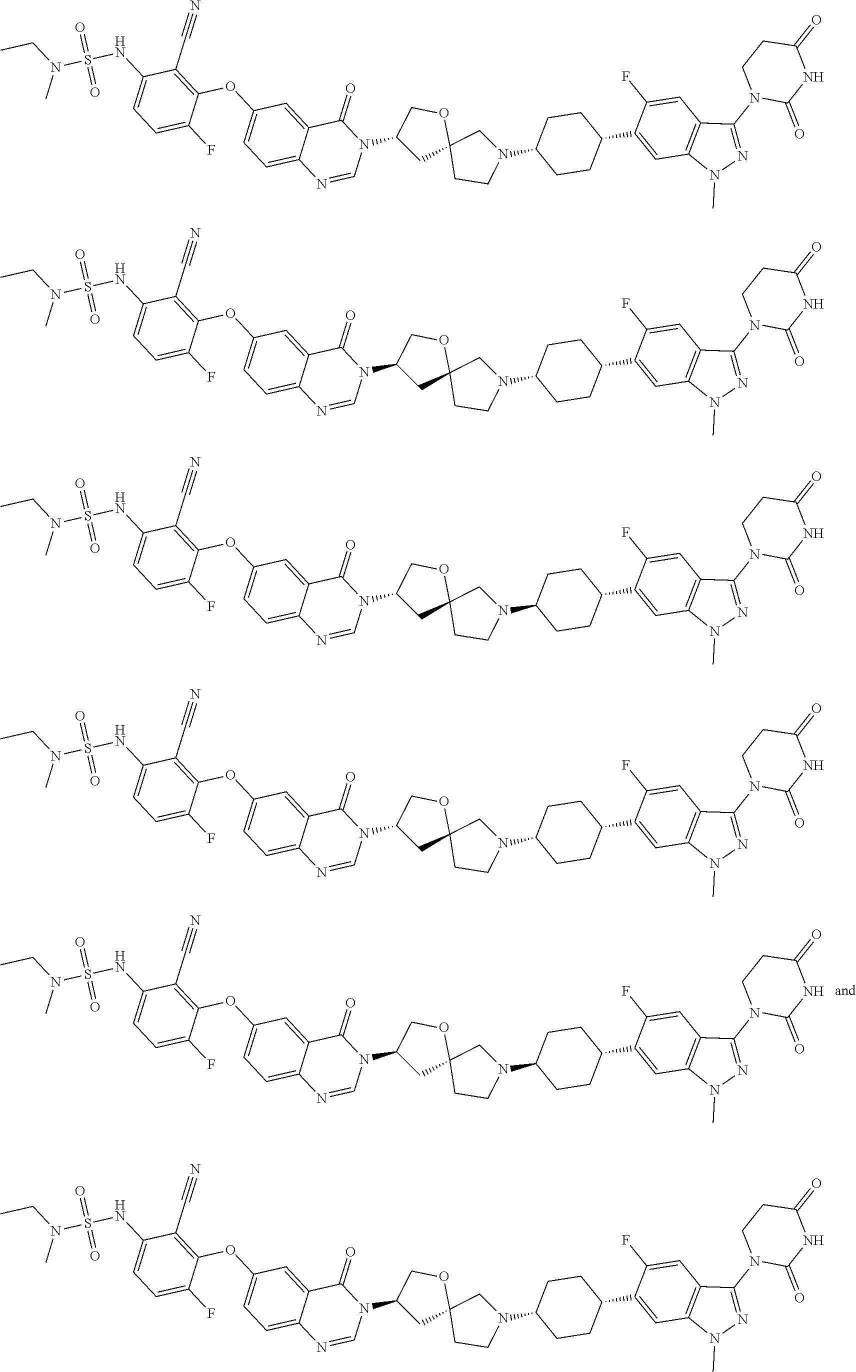

or pharmaceutically acceptable salt thereof.

The invention further provides the following.

A compound of Formula XII or Formula XXVIII or a pharmaceutically acceptable salt thereof, for use as a therapeutically active substance in the treatment of a mutant BRAF mediated disorder.

A pharmaceutical composition comprising a compound of Formula XII or Formula XXVIII or a pharmaceutically acceptable salt thereof, and a pharmaceutically acceptable carrier.

The use of a compound of Formula XII or Formula XXVIII or a pharmaceutically acceptable salt thereof, for the therapeutic and/or prophylactic treatment of cancer.

A compound of Formula XII or Formula XXVIII or a pharmaceutically acceptable salt thereof, for use in the treatment and/or prophylaxis of cancer.

The use of a compound of Formula XII or Formula XXVIII or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula XII or Formula XXVIII or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula XIII and Formula XXIX

In certain embodiments a compound of the invention is selected from the compound of Formula XIII and the compound of Formula XXIX:

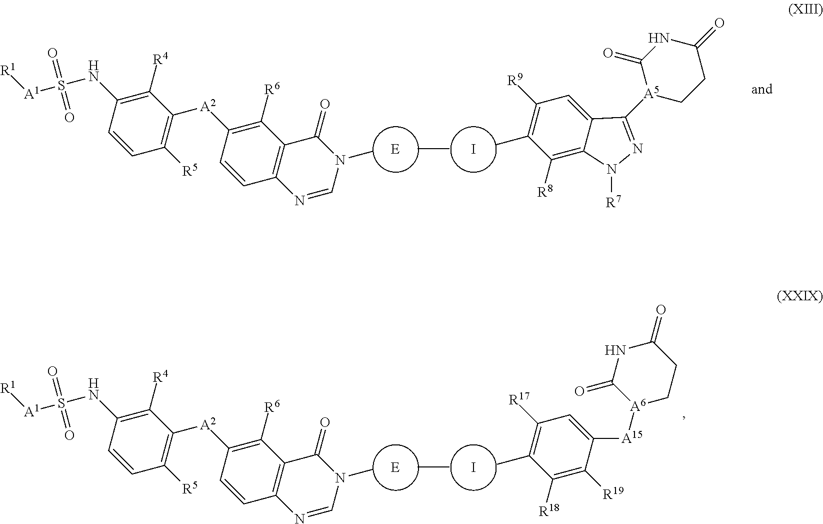

or a pharmaceutically acceptable salt thereof,
wherein:
Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
Cycle \textcircled{i} is a cycloalkyl, wherein cycle \textcircled{I} is optionally substituted with one or two substituents independently selected from halogen, alkyl and alkoxy; and all other substituents are as defined herein.

In certain embodiments the compound of Formula XIII or Formula XXIX is selected from:

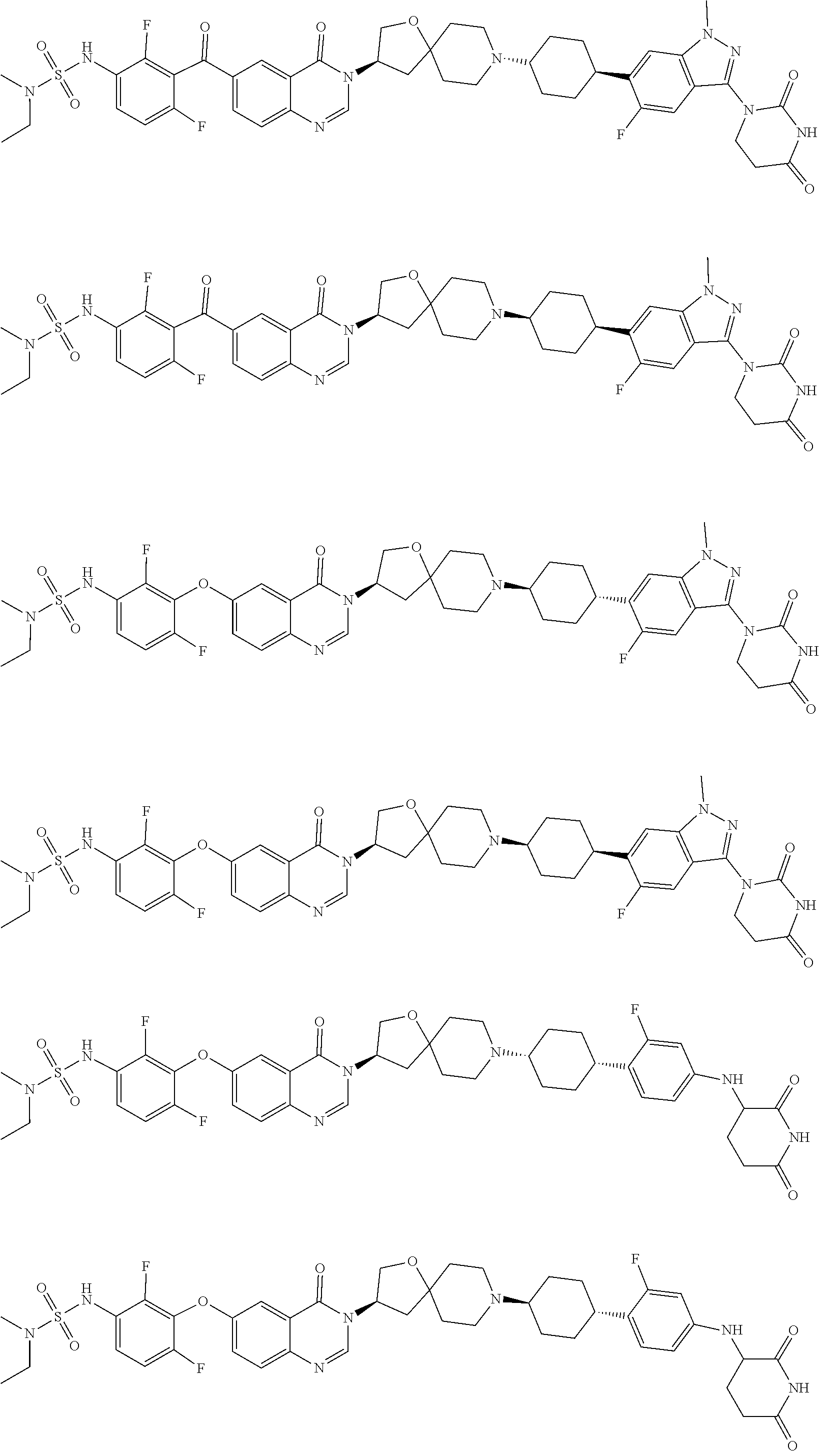

or pharmaceutically acceptable salt thereof.

The invention further provides the following.

A compound of Formula XIII or Formula XXIX or a pharmaceutically acceptable salt thereof, for use as a therapeutically active substance in the treatment of a mutant BRAF mediated disorder.

A pharmaceutical composition comprising a compound of Formula XIII or Formula XXIX or a pharmaceutically acceptable salt thereof, and a pharmaceutically acceptable carrier.

The use of a compound of Formula XIII or Formula XXIX or a pharmaceutically acceptable salt thereof, for the therapeutic and/or prophylactic treatment of cancer.

A compound of Formula XIII or Formula XXIX or a pharmaceutically acceptable salt thereof, for use in the treatment and/or prophylaxis of cancer.

The use of a compound of Formula XIII or Formula XXIX or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula XIII or Formula XXIX or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula XIV and Formula XXX

In certain embodiments a compound of the invention is selected from the compound of Formula XIV and the compound of Formula XXX:

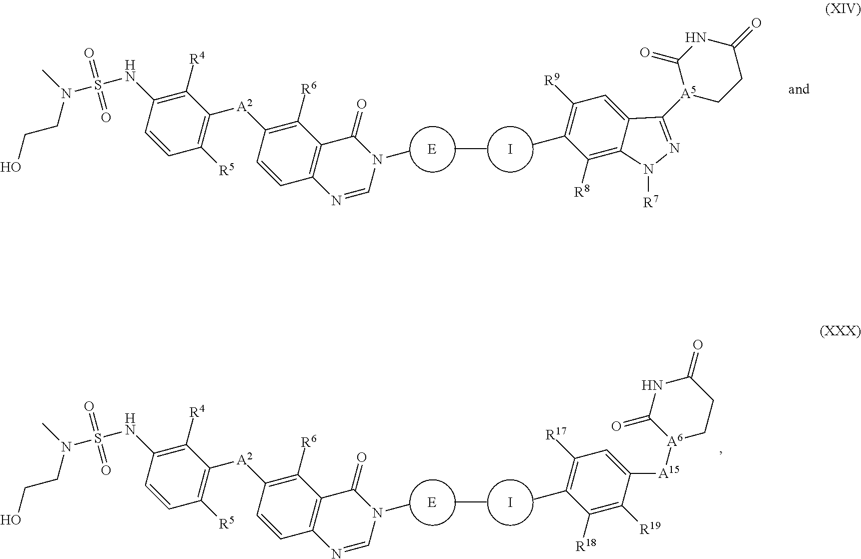

or a pharmaceutically acceptable salt thereof,
wherein
Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
Cycle \textcircled{I} is a cycloalkyl, wherein cycle \textcircled{I} is optionally substituted with one or two substituents independently selected from halogen, alkyl and alkoxy; and all other substituents are as defined herein.

In certain embodiments the compound of Formula XIV or Formula XXX is selected from:

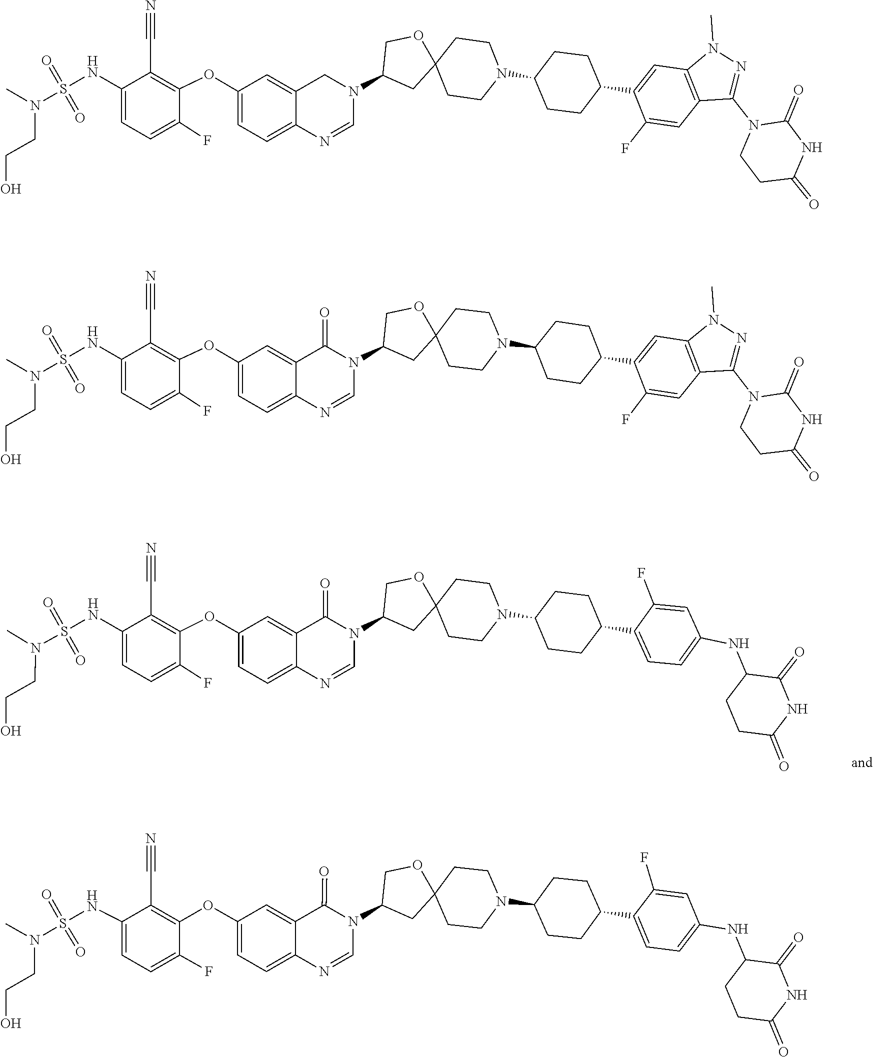

or a pharmaceutically acceptable salt thereof.

The invention further provides the following.

A compound of Formula XIV or Formula XXX or a pharmaceutically acceptable salt thereof, for use as a therapeutically active substance in the treatment of a mutant BRAF mediated disorder.

A pharmaceutical composition comprising a compound of Formula XIV or Formula XXX or a pharmaceutically acceptable salt thereof, and a pharmaceutically acceptable carrier.

The use of a compound of Formula XIV or Formula XXX or a pharmaceutically acceptable salt thereof, for the therapeutic and/or prophylactic treatment of cancer.

A compound of Formula XIV or Formula XXX or a pharmaceutically acceptable salt thereof, for use in the treatment and/or prophylaxis of cancer.

The use of a compound of Formula XIV or Formula XXX or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula XIV or Formula XXX or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula XV and Formula XXXI

In certain embodiments a compound of the invention is selected from the compound of Formula XV and the compound of Formula XXXI:

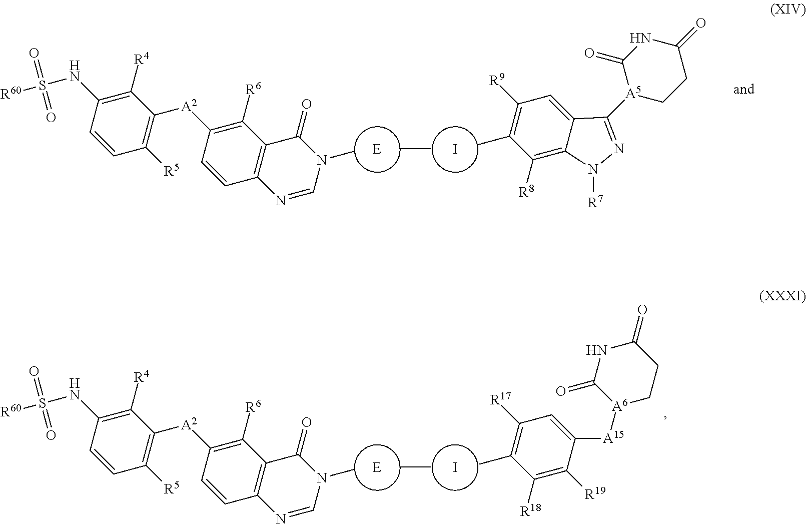

or a pharmaceutically acceptable salt thereof,
wherein
Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
Cycle \textcircled{I} is a cycloalkyl, wherein cycle \textcircled{I} is optionally substituted with one or two substituents independently selected from halogen, alkyl and alkoxy; and all other substituents are as defined herein.

In certain embodiments the compound of Formula XV or Formula XXXI is selected from:

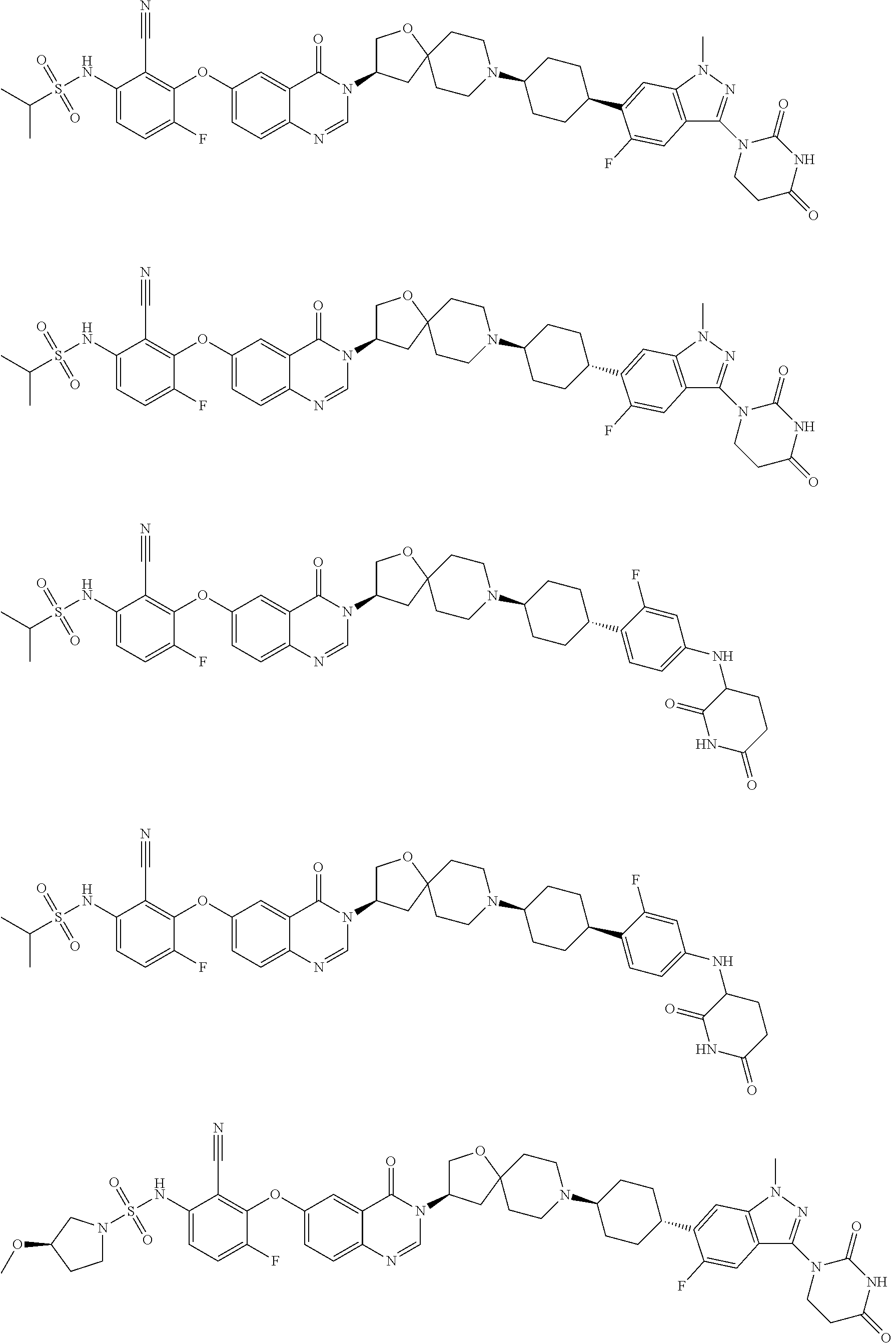

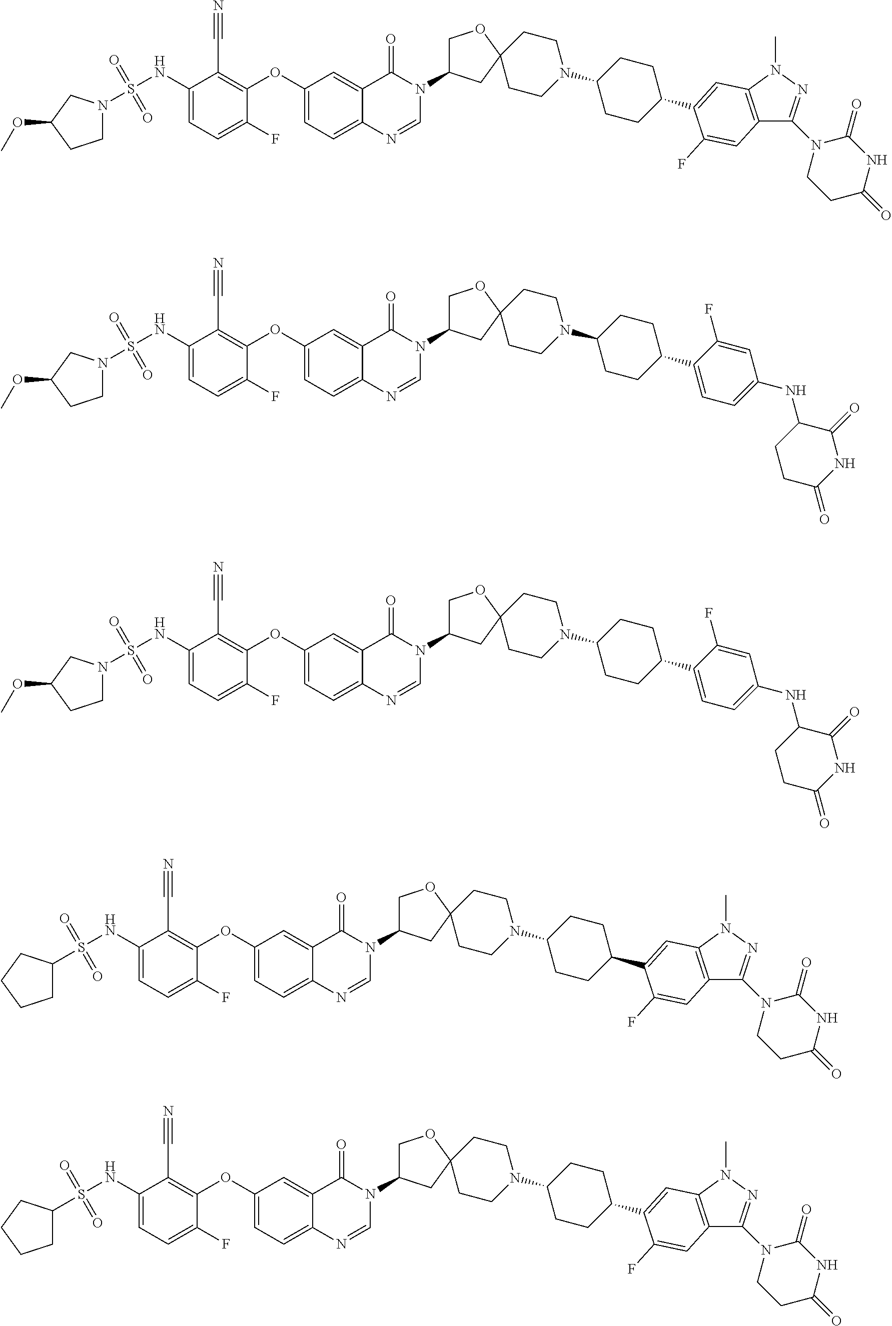

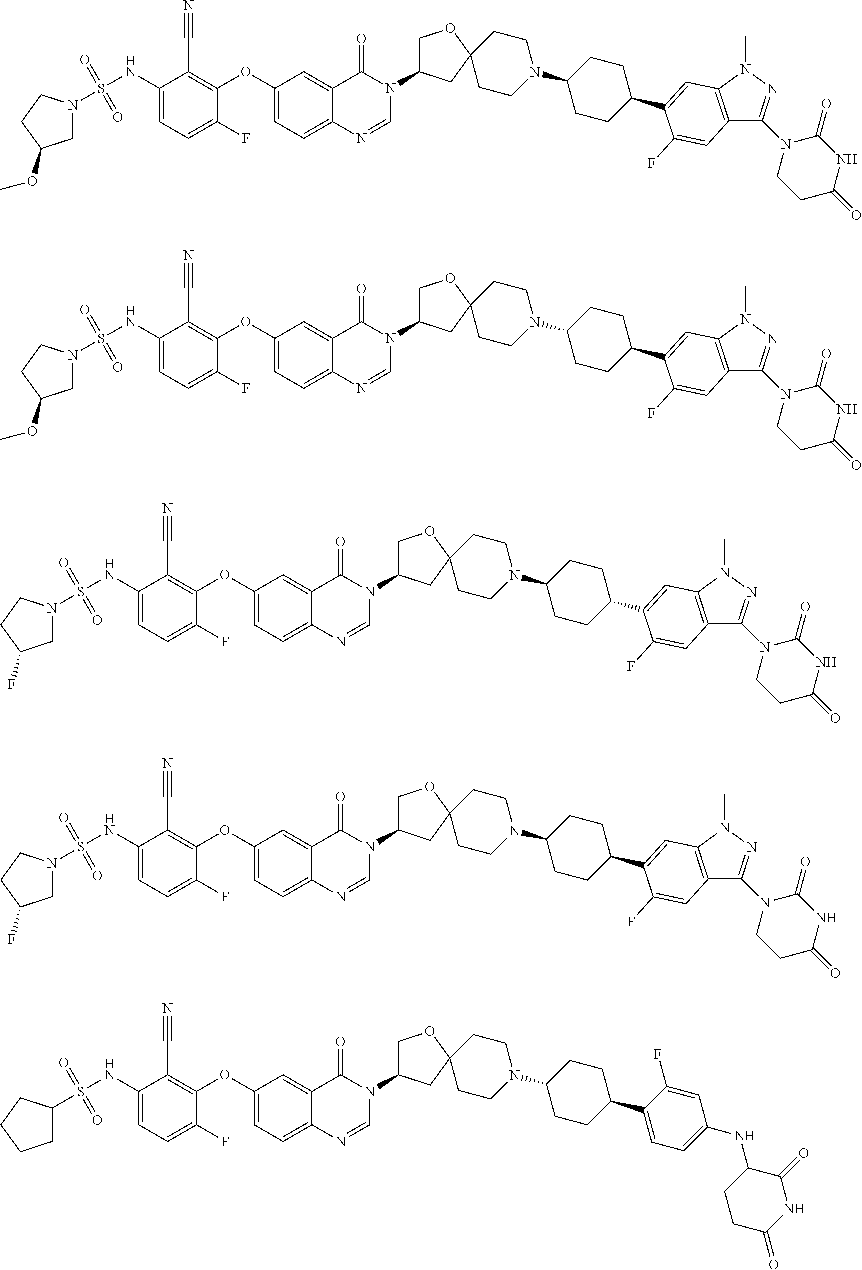

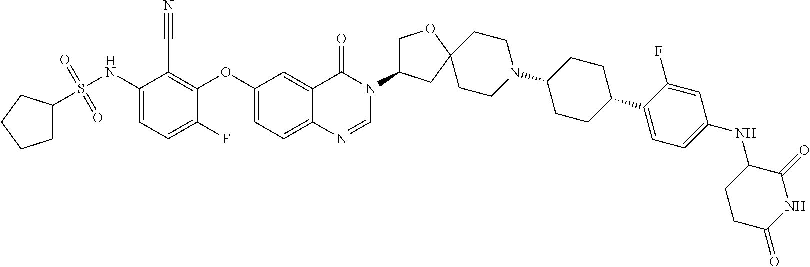 [SMILES: N#Cc1c(NS(=O)(=O)C2CCCC2)ccc(F)c1Oc1ccc2ncn([C@H]3COC4(CCN([C@H]5CC[C@@H](c6ccc(NC7CCC(=O)NC7=O)cc6F)CC5)CC4)C3)c(=O)c2c1]

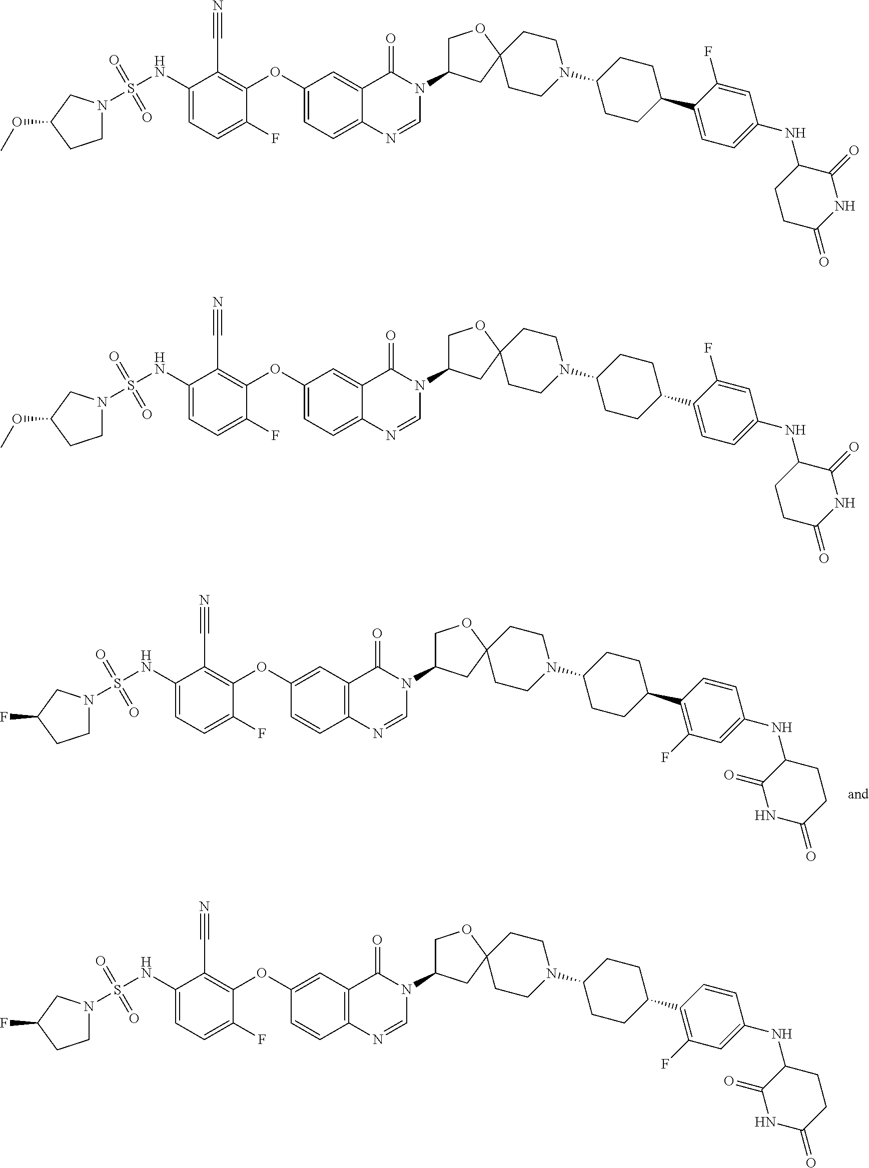

or a pharmaceutically acceptable salt thereof.

The invention further provides the following.

A compound of Formula XV or Formula XXXI or a pharmaceutically acceptable salt thereof, for use as a therapeutically active substance in the treatment of a mutant BRAF mediated disorder.

A pharmaceutical composition comprising a compound of Formula XV or Formula XXXI or a pharmaceutically acceptable salt thereof, and a pharmaceutically acceptable carrier.

The use of a compound of Formula XV or Formula XXXI or a pharmaceutically acceptable salt thereof, for the therapeutic and/or prophylactic treatment of cancer.

A compound of Formula XV or Formula XXXI or a pharmaceutically acceptable salt thereof, for use in the treatment and/or prophylaxis of cancer.

The use of a compound of Formula XV or Formula XXXI or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula XV or Formula XXXI or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

### Embodiments of Formula XVI and Formula XXXII

In certain embodiments a compound of the invention is selected from the compound of Formula XVI and the compound of Formula XXXII:

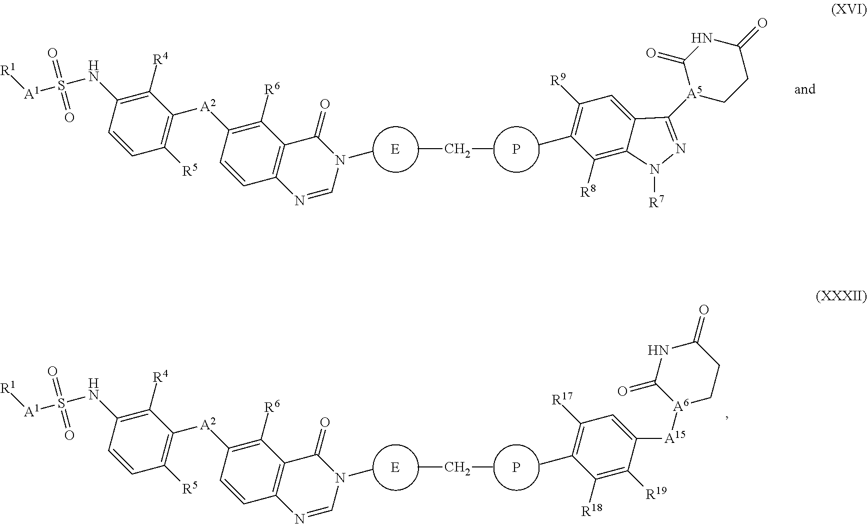

or a pharmaceutically acceptable salt thereof,
wherein
Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
Cycle \textcircled{P} is a heterocyclyl optionally substituted with one or two substituents independently selected from halogen, alkyl OH and alkoxy; and all other substituents are as defined herein.

In certain embodiments the compound of Formula XVI or Formula XXXII is selected from

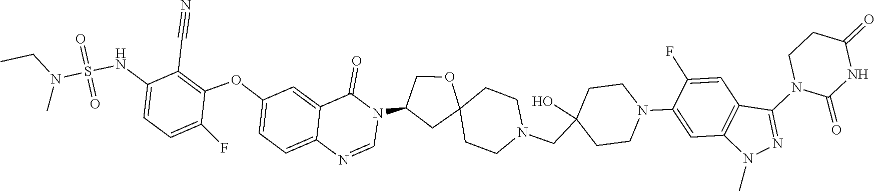 [SMILES: CCN(C)S(=O)(=O)Nc1ccc(F)c(Oc2ccc3ncn([C@H]4COC5(CCN(CC6(O)CCN(c7cc8c(cc7F)c(N7CCC(=O)NC7=O)nn8C)CC6)CC5)C4)c(=O)c3c2)c1C#N]

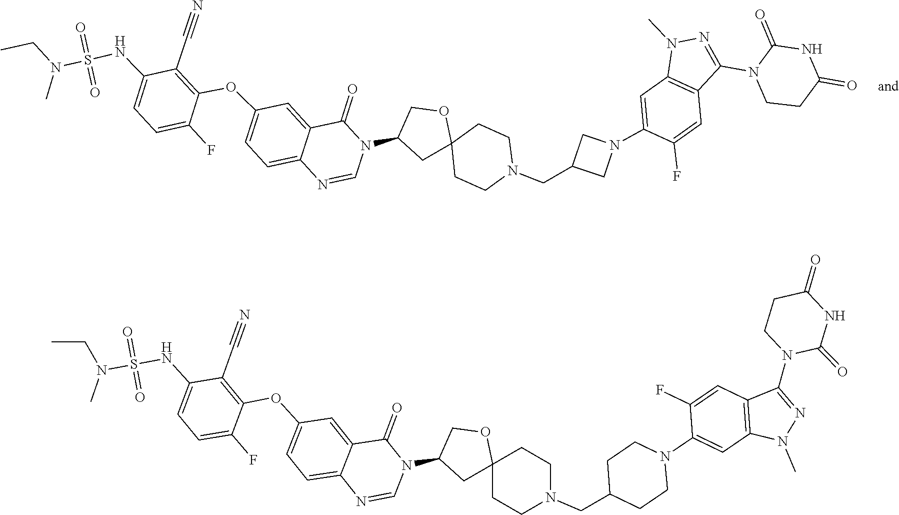 [SMILES: CCN(C)S(=O)(=O)Nc1ccc(F)c(Oc2ccc3ncn([C@H]4COC5(CCN(CC6CN(c7cc8c(cc7F)c(N7CCC(=O)NC7=O)nn8C)C6)CC5)C4)c(=O)c3c2)c1C#N ; CCN(C)S(=O)(=O)Nc1ccc(F)c(Oc2ccc3ncn([C@H]4COC5(CCN(CC6CCN(c7cc8c(cc7F)c(N7CCC(=O)NC7=O)nn8C)CC6)CC5)C4)c(=O)c3c2)c1C#N]

or a pharmaceutically acceptable salt thereof.

The invention further provides the following.

A compound of Formula XVI or Formula XXXII or a pharmaceutically acceptable salt thereof, for use as a therapeutically active substance in the treatment of a mutant BRAF mediated disorder.

A pharmaceutical composition comprising a compound of Formula XVI or Formula XXXII or a pharmaceutically acceptable salt thereof, and a pharmaceutically acceptable carrier.

The use of a compound of Formula XVI or Formula XXXII or a pharmaceutically acceptable salt thereof, for the therapeutic and/or prophylactic treatment of cancer.

A compound of Formula XVI or Formula XXXII or a pharmaceutically acceptable salt thereof, for use in the treatment and/or prophylaxis of cancer.

The use of a compound of Formula XVI or Formula XXXII or a pharmaceutically acceptable salt thereof, for the preparation of a medicament for the therapeutic and/or prophylactic treatment of cancer.

A method for the therapeutic and/or prophylactic treatment of cancer, which method comprises administering an effective amount of a compound of Formula XVI or Formula XXXII or a pharmaceutically acceptable salt thereof, to a patient in need thereof.

In some embodiments the cancer is a BRAF V600X mutated tumor.

In some embodiments the cancer is a BRAF V600E/K mutated tumor.

In some embodiments the cancer is targeted therapy naïve.

In some embodiments the cancer is selected from melanoma, colorectal cancer and lung cancer, in particular non-small cell lung cancer.

Additional embodiments of a compound of the invention:

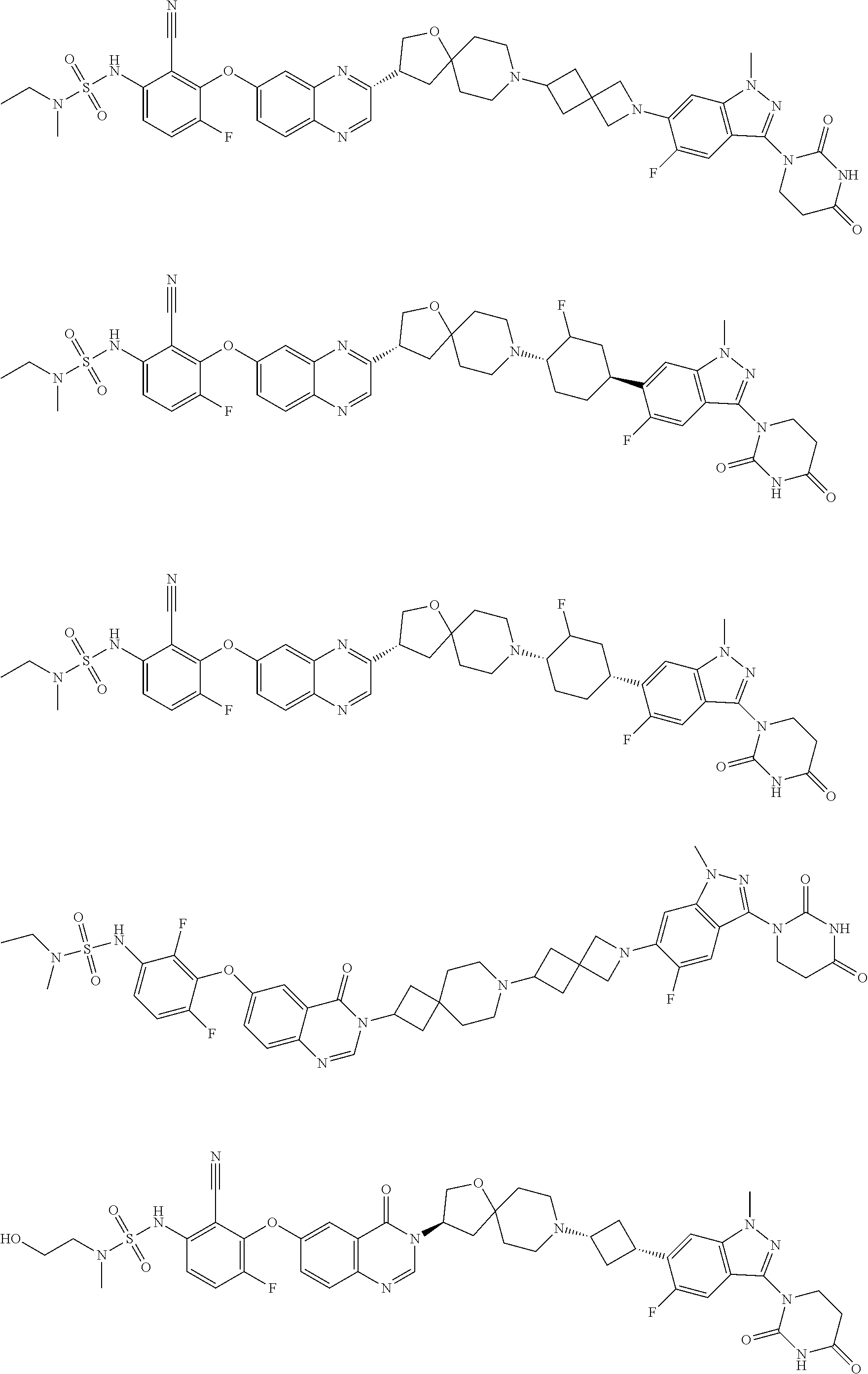

### Embodiments of Formula I to Formula XXXII

1. A compound of Formula:

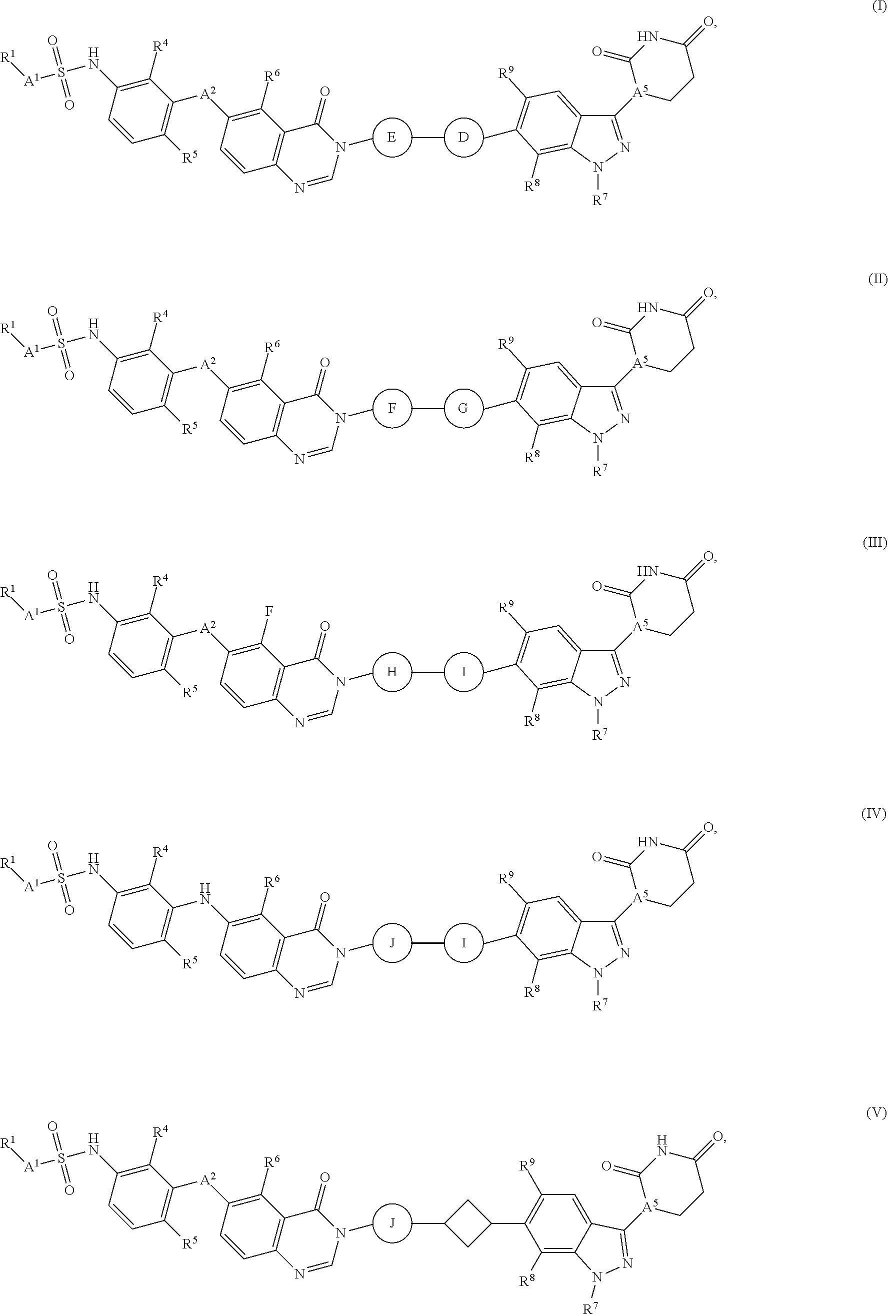

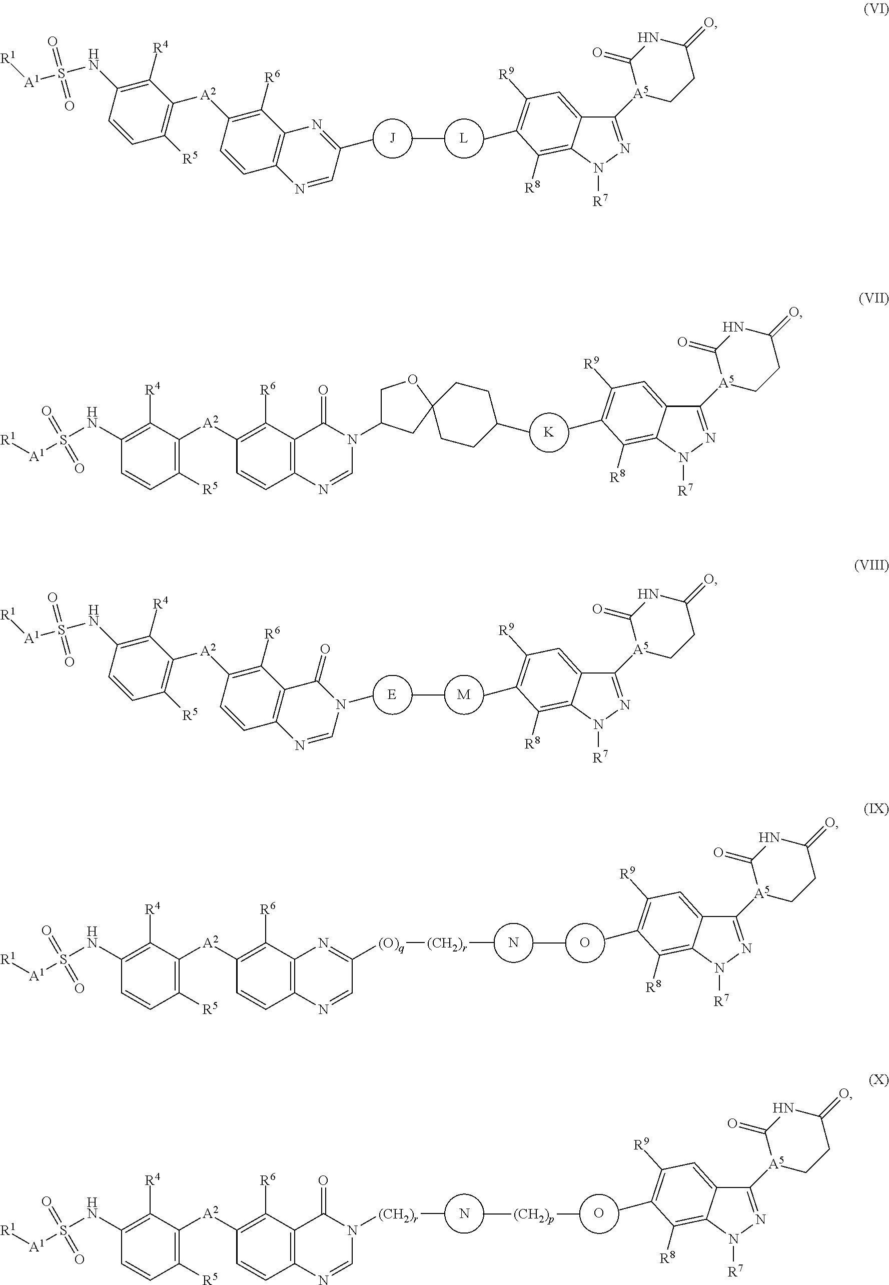

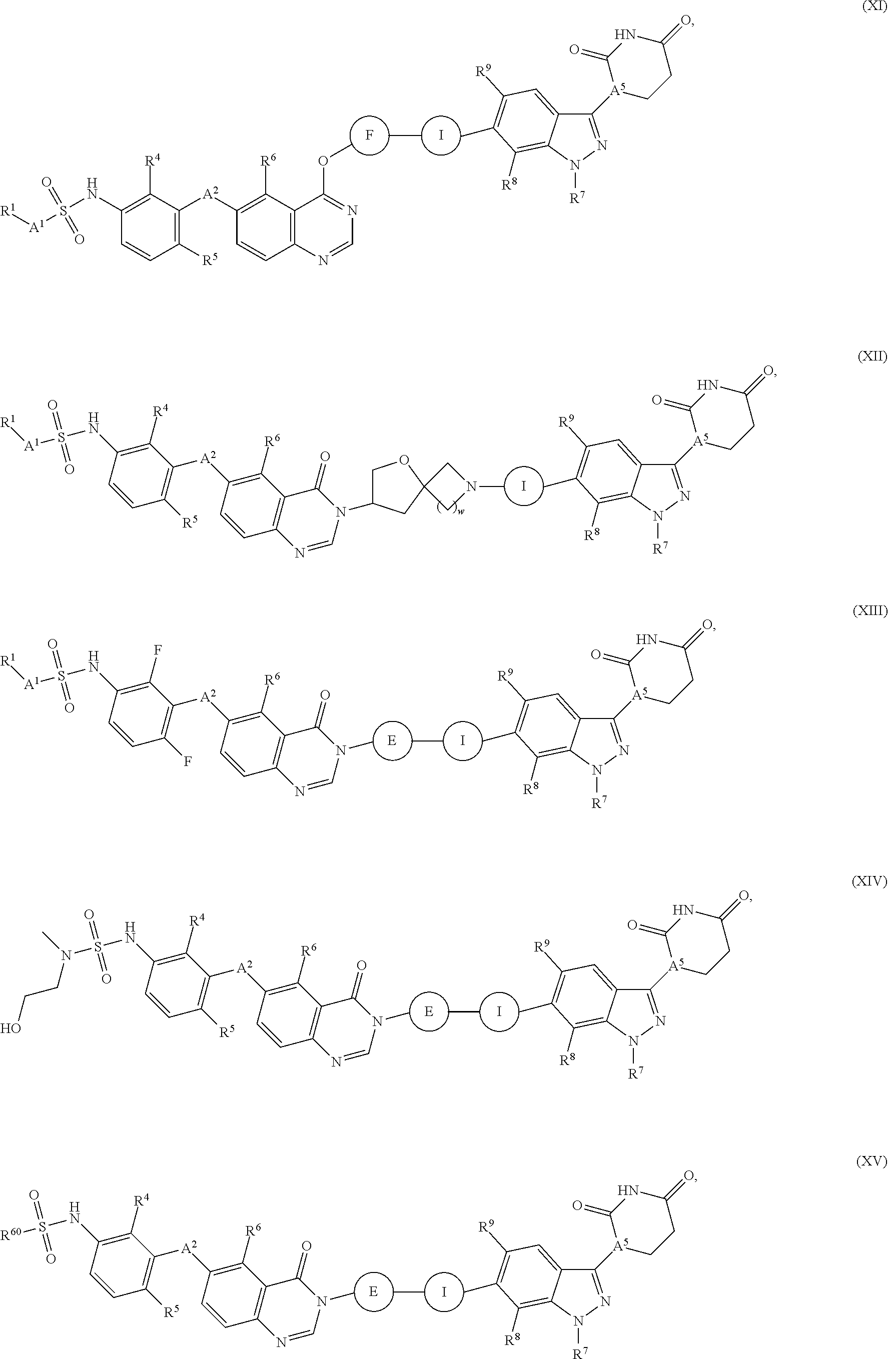

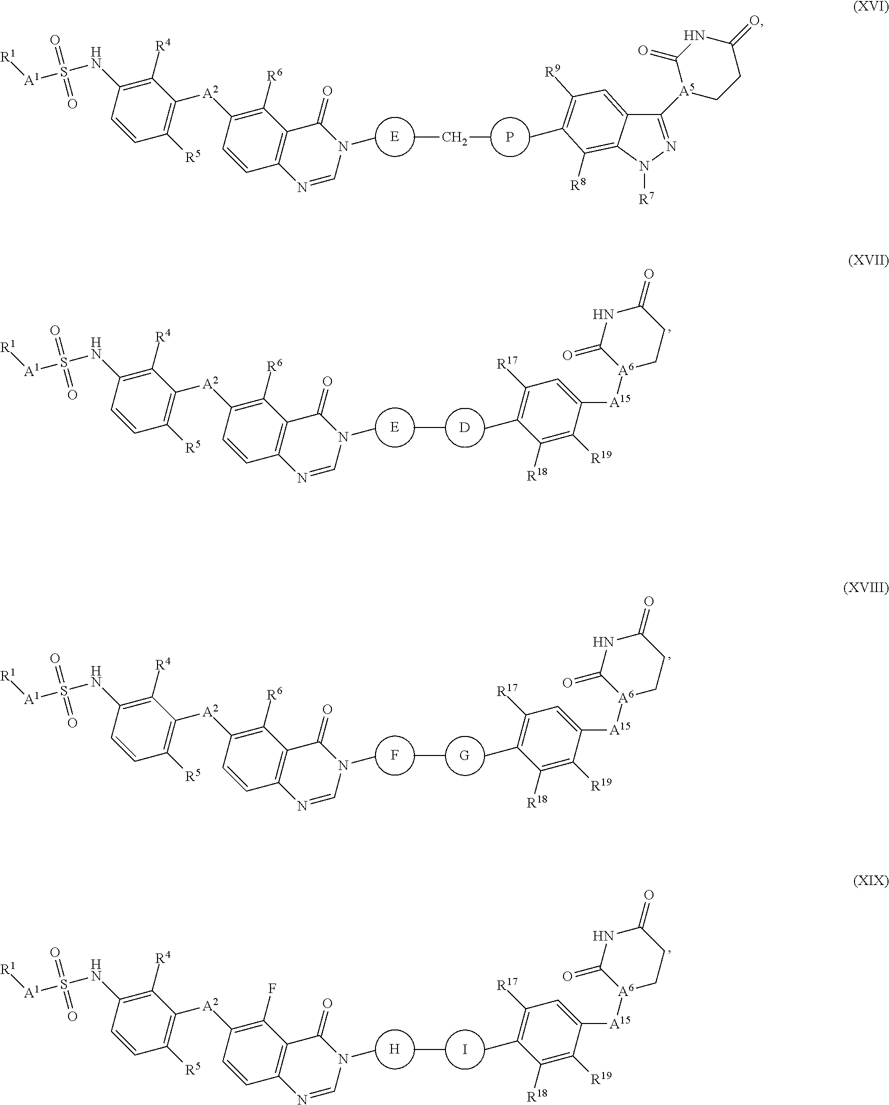

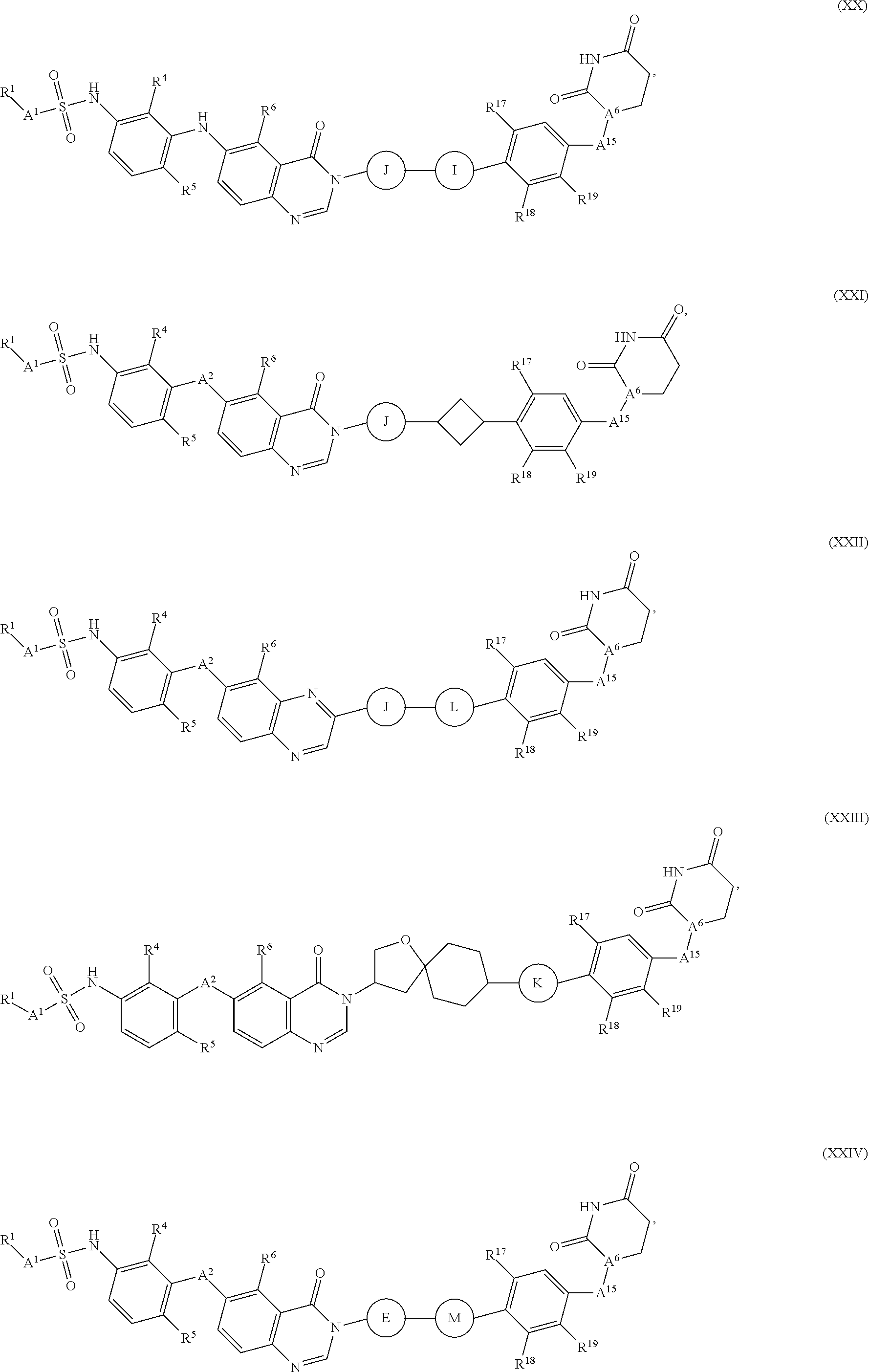

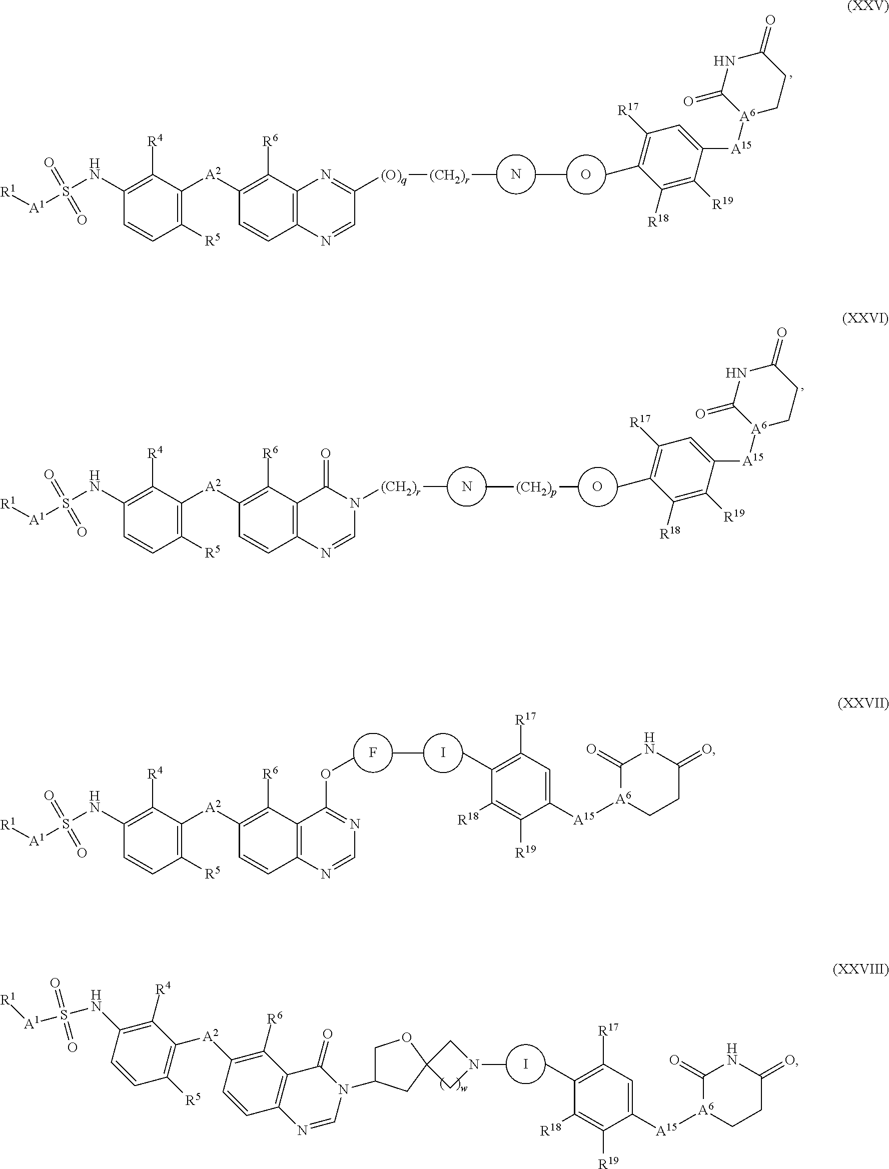

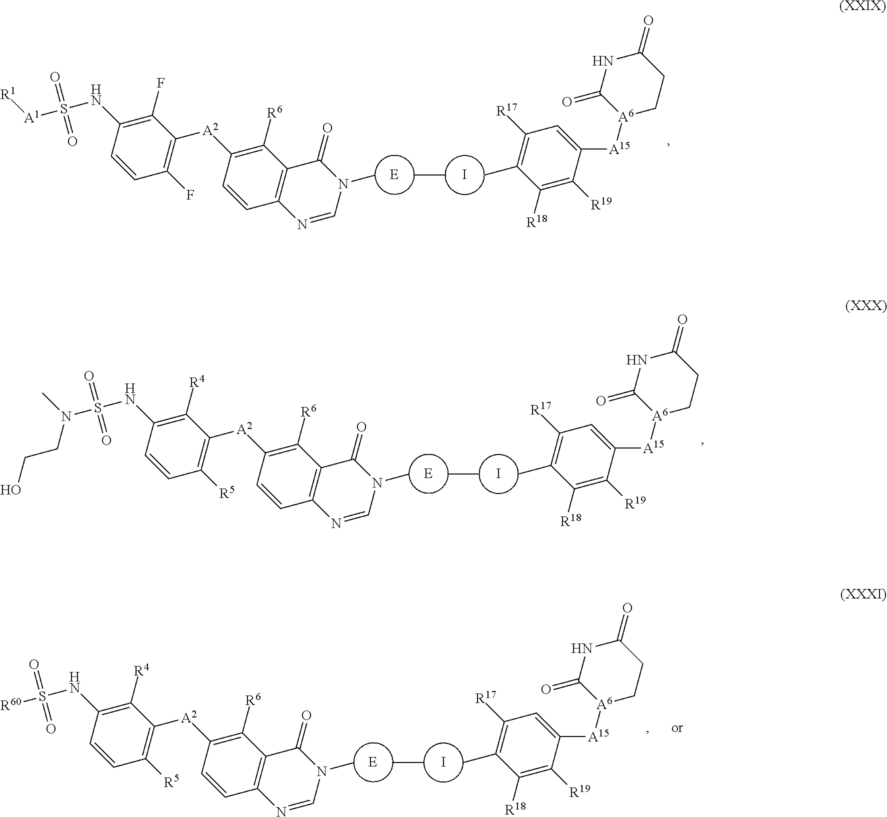

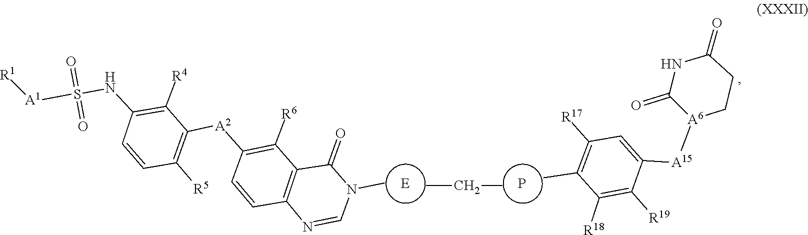

or a pharmaceutically acceptable salt thereof,
wherein
Cycle \textcircled{D} is a spiroheterocyclyl containing 1 or 2 nitrogen atoms and containing no other heteroatoms or piperidine-1,4-diyl, wherein cycle \textcircled{D} is optionally substituted with one or two substituents independently selected from halogen, alkyl and alkoxy;
Cycle \textcircled{E} is a spiroheterocyclyl containing 1 nitrogen atom and 1 oxygen atom and containing no other heteroatoms wherein cycle \textcircled{E} is optionally substituted with one or two substituents independently selected from halogen, alkyl and alkoxy;
Cycle \textcircled{F} is a bridged heterocyclyl or spiroheterocyclyl containing 1 or 2 nitrogen atoms and containing no other heteroatoms, wherein cycle \textcircled{D} is optionally substituted with one or two substituents independently selected from halogen, alkyl and alkoxy;
Cycle \textcircled{G} is a cycloalkyl or heterocyclyl containing 1 or 2 nitrogen atoms and 0 or 1 oxygen atom and containing no other heteroatoms, wherein cycle \textcircled{G} is optionally substituted with one or two substituents independently selected from halogen, alkyl and alkoxy;
Cycle \textcircled{H} is a spiroheterocyclyl containing 1 oxygen atom and 0 or 1 nitrogen atom and containing no other heteroatoms, wherein cycle \textcircled{H} is optionally substituted with one or two substituents independently selected from halogen, alkyl and alkoxy;
Cycle \textcircled{I} is a cycloalkyl, wherein cycle \textcircled{I} is optionally substituted with one or two substituents independently selected from halogen, alkyl and alkoxy;
Cycle \textcircled{J} is a spiroheterocyclyl containing 1 or 2 nitrogen atoms and 0 or 1 oxygen atom and containing no other heteroatoms, wherein cycle \textcircled{J} is optionally substituted with one or two substituents independently selected from halogen, alkyl and alkoxy;
Cycle \textcircled{K} is a cycloalkyl, 3,3-difluoro-piperidine-1,4-diyl, piperazine-1,4-diyl, or a spiroheterocyclyl containing 1 or 2 nitrogen atoms and 0 or 1 oxygen atom and containing no other heteroatoms, wherein the cycloalkyl piperazine-1,4-diyl, or a spiroheterocyclyl is optionally substituted with one or two substituents independently selected from halogen, alkyl and alkoxy;
Cycle \textcircled{L} is a cycloalkyl, piperidine-1,4-diyl, spiroheterocyclyl containing 1 or 2 nitrogen atoms and containing no other heteroatoms, wherein cycle \textcircled{L} is optionally substituted with one or two substituents independently selected from halogen, alkyl and alkoxy;
Cycle \textcircled{M} is a cycloalkyl substituted with 1, 2, or 3 substituents independently selected from the group consisting of alkyl, alkoxy, F, Cl, Br, and OH;
Cycle \textcircled{N} is a cycloalkyl, spirocycloalkyl, heterocyclyl containing 1 or 2 nitrogen atoms and containing no other heteroatoms, wherein cycle \textcircled{N} is optionally substituted with one or two substituents independently selected from halogen, alkyl and alkoxy;
Cycle \textcircled{O} is a cycloalkyl, spirocycloalkyl, heterocyclyl containing 1 or 2 nitrogen atoms and containing no other heteroatoms, wherein cycle \textcircled{O} is optionally substituted with one or two substituents independently selected from halogen, alkyl and alkoxy;
Cycle \textcircled{P} is a heterocyclyl optionally substituted with one or two substituents independently selected from halogen, alkyl OH and alkoxy;
A¹ is selected from —NR²— and —CHR^{2′}—;
R¹ is selected from hydrogen, alkyl and cycloalkyl;
R² is selected from hydrogen, alkyl, cycloalkyl and haloalkyl;
or R¹ and R² together with the nitrogen atom to which they are attached form heterocycloalkyl optionally substituted with one or two R³;
R^{2′} is selected from hydrogen, alkyl, cycloalkyl and haloalkyl;
or R¹ and R^{2′} together with the carbon atom to which they are attached form cycloalkyl optionally substituted with one or two R³;
each R³ is independently selected from hydrogen, halogen, alkyl, cycloalkyl and alkoxy;
R⁴ is selected from hydrogen, alkyl, cyano and halogen;
R⁵ is selected from hydrogen, alkyl, cyano and halogen;
A² is selected from —O—, —NH— and —(C═O)—;
R⁶ is selected from hydrogen, halogen, hydroxy, amino, dialkylamino, alkoxy, alkyl and alkoxyalkyl;
R⁷ is selected from hydrogen and alkyl;
R⁸ is selected from hydrogen, alkyl, cyano, halogen, and alkoxy;
R⁹ is selected from hydrogen, alkyl, cyano, halogen, and alkoxy;
R¹⁷ is selected from hydrogen, alkyl, cyano, hydroxy, cycloalkyl, halogen and alkoxy;
R¹⁸ is selected from hydrogen, alkyl, cyano, hydroxy, cycloalkyl, halogen and alkoxy;
R¹⁹ is selected from hydrogen, alkyl, cyano, hydroxy, cycloalkyl, halogen and alkoxy;
A⁵ is —CH— or —N—;
A¹⁵ is selected from a bond, —O— and —NH—, wherein A⁶ is CH when A¹⁵ is O or NH and wherein A⁶ is N or CH when A¹⁵ is bond;
R⁶⁰ is isopropyl; pyrrolidine-1-yl substituted with F or OMe; or cyclopropyl;
q is 0 or 1;
r is 0, 1 or 2;
p is 0, 1 or 2; and
w is 1 or 2.

2. The compound of embodiment 1, wherein the compound is of Formula

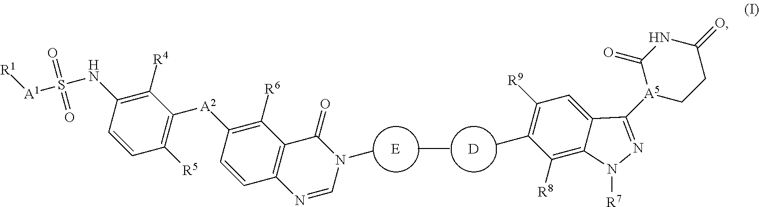

or a pharmaceutically acceptable salt thereof.

3. The compound of embodiment 1, wherein the compound is of Formula

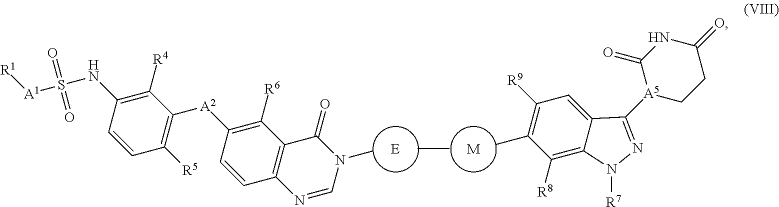

or a pharmaceutically acceptable salt thereof.

4. The compound of embodiment 1, wherein the compound is of Formula

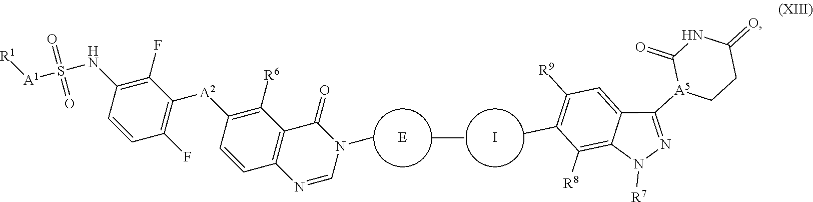

or a pharmaceutically acceptable salt thereof.

5. The compound of embodiment 1, wherein the compound is of Formula

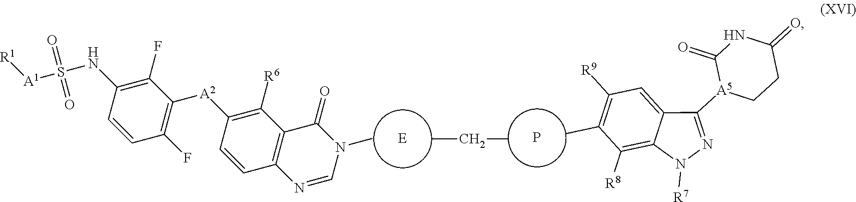

or a pharmaceutically acceptable salt thereof.

6. The compound of embodiment 1, wherein the compound is of Formula

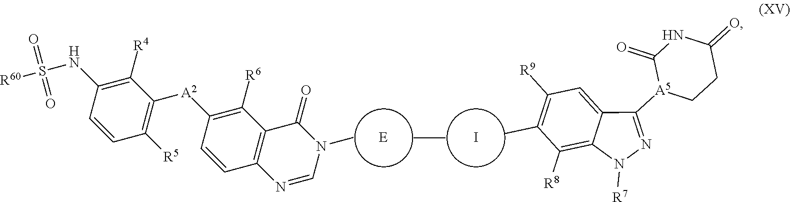

or a pharmaceutically acceptable salt thereof.

7. The compound of embodiment 1, wherein the compound is of Formula

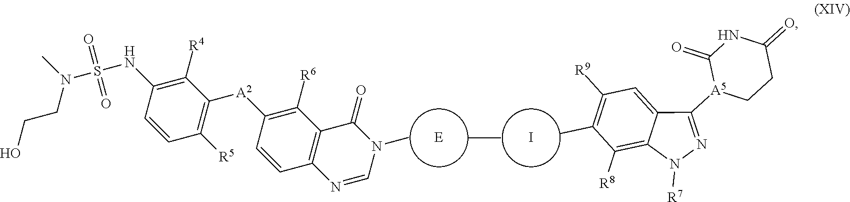

or a pharmaceutically acceptable salt thereof.

8. The compound of any one of embodiments 1-7, wherein Cycle \textcircled{E} is

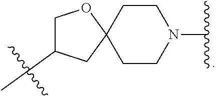 [SMILES: *C1COC2(CCN(*)CC2)C1]

9. The compound of any one of embodiments 1-7, wherein Cycle \textcircled{E} is

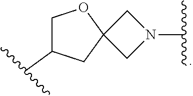 [SMILES: *C1COC2(C1)CN(*)C2]

10. The compound of embodiment 1, wherein the compound is of Formula

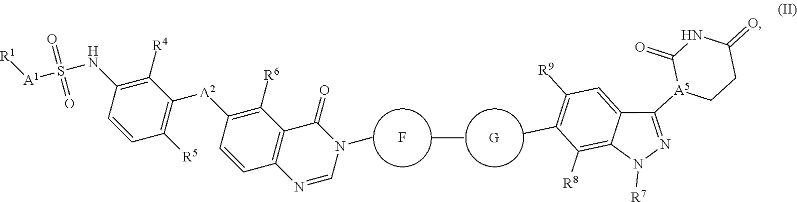

or a pharmaceutically acceptable salt thereof.

11. The compound of embodiment 1, wherein the compound is of Formula

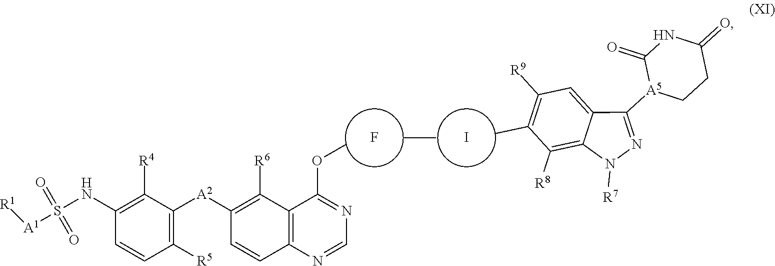

or a pharmaceutically acceptable salt thereof.

12. The compound of embodiment 10 or 11, wherein Cycle \textcircled{F} is

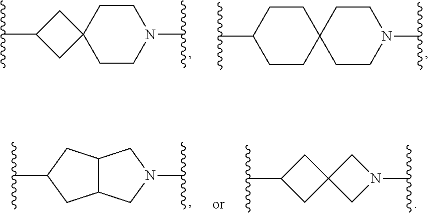

13. The compound of embodiment 10 or 11, wherein Cycle \textcircled{F} is

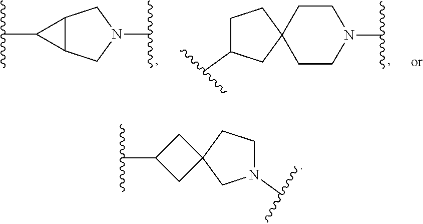 [SMILES: *C1C2CN(*)CC12 ; *C1CCC2(CCN(*)CC2)C1 ; *C1CC2(CCN(*)C2)C1]

14. The compound of embodiment 1, wherein the compound is of Formula

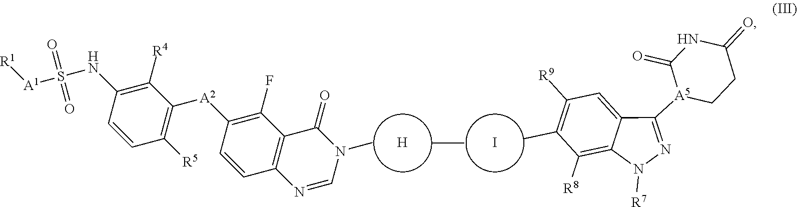

or a pharmaceutically acceptable salt thereof.

15. The compound of embodiment 1, wherein the compound is of Formula

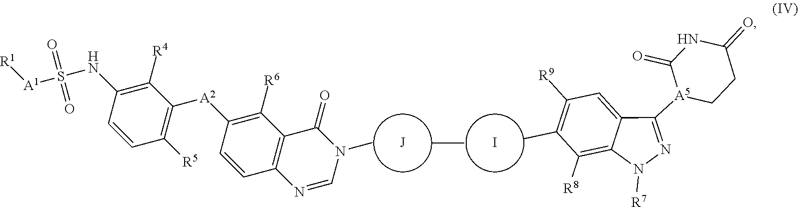

or a pharmaceutically acceptable salt thereof.

16. The compound of embodiment 1, wherein the compound is of Formula

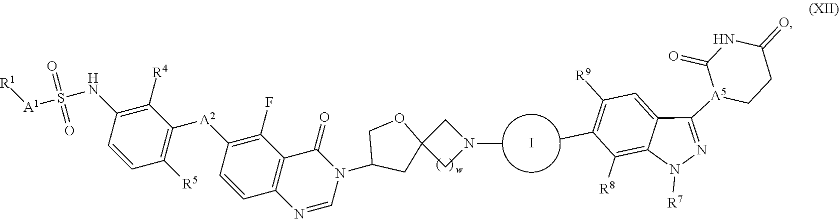

or a pharmaceutically acceptable salt thereof.

17. The compound of any one of embodiments 14-16, wherein Cycle \textcircled{I} is

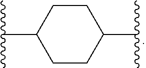 [SMILES: *C1CCC(*)CC1]

18. The compound of embodiment 1, wherein the compound is of Formula

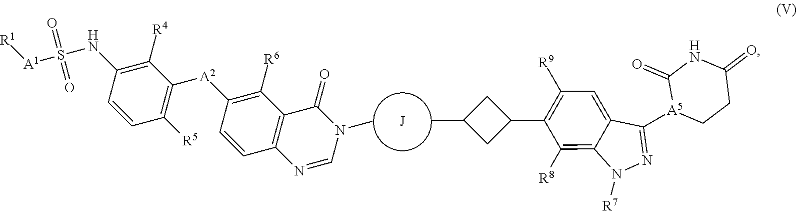

or a pharmaceutically acceptable salt thereof.

19. The compound of embodiment 1, wherein the compound is of Formula

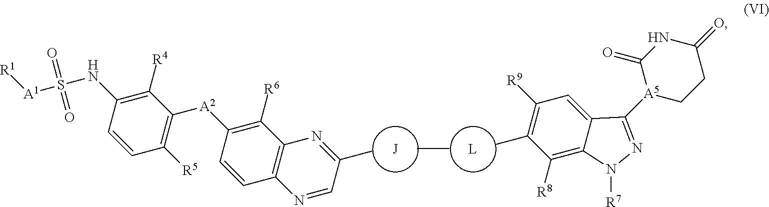

or a pharmaceutically acceptable salt thereof.

20. The compound of embodiment 1, wherein the compound is of Formula

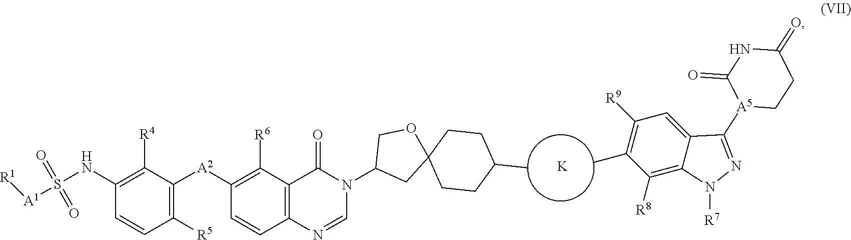

or a pharmaceutically acceptable salt thereof.

21. The compound of embodiment 1, wherein the compound is of Formula

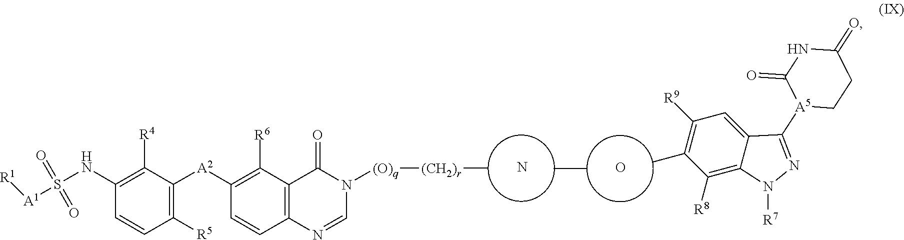

or a pharmaceutically acceptable salt thereof.

22. The compound of embodiment 1, wherein the compound is of Formula

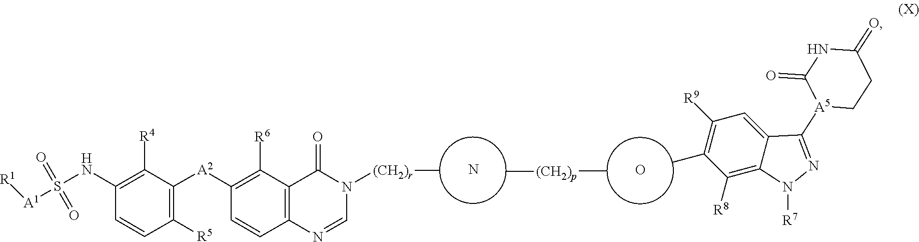

or a pharmaceutically acceptable salt thereof.

23. The compound of any one of embodiments 1-22 wherein A⁵ is —CH—.

24. The compound of any one of embodiments 1-22 wherein A⁵ is —N—.

25. The compound of any one of embodiments 1-24 wherein R⁷ is CH₃.

26. The compound of any one of embodiments 1-24 wherein R⁷ is H.

27. The compound of any one of embodiments 1-26 wherein R⁸ is CH₃.

28. The compound of any one of embodiments 1-26 wherein R⁸ is H.

29. The compound of any one of embodiments 1-26 wherein R⁸ is F.

30. The compound of any one of embodiments 1-26 wherein R⁸ is CN.

31. The compound of any one of embodiments 1-26 wherein R⁸ is OCH₃.

32. The compound of any one of embodiments 1-31 wherein R⁹ is CH₃.

33. The compound of any one of embodiments 1-31 wherein R⁹ is H.

34. The compound of any one of embodiments 1-31 wherein R⁹ is F.

35. The compound of any one of embodiments 1-31 wherein R⁹ is CN.

36. The compound of any one of embodiments 1-31 wherein R⁹ is OCH₃.

37. The compound of embodiment 1, wherein the compound is of Formula

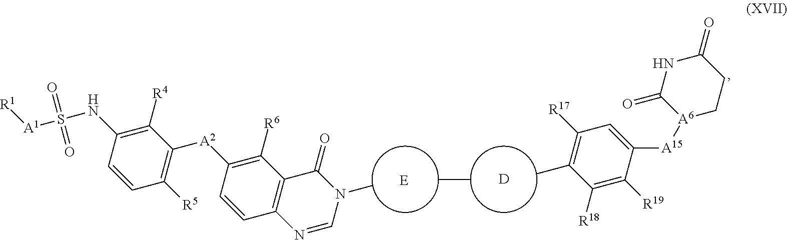

or a pharmaceutically acceptable salt thereof.

38. The compound of embodiment 1, wherein the compound is of Formula

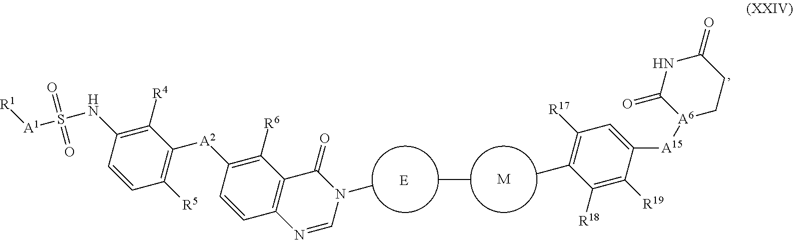

or a pharmaceutically acceptable salt thereof.

39. The compound of embodiment 1, wherein the compound is of Formula

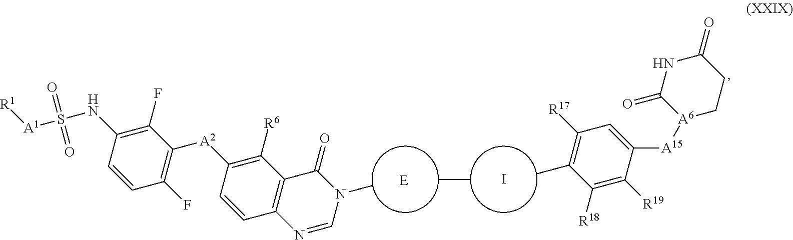

or a pharmaceutically acceptable salt thereof.

40. The compound of embodiment 1, wherein the compound is of Formula

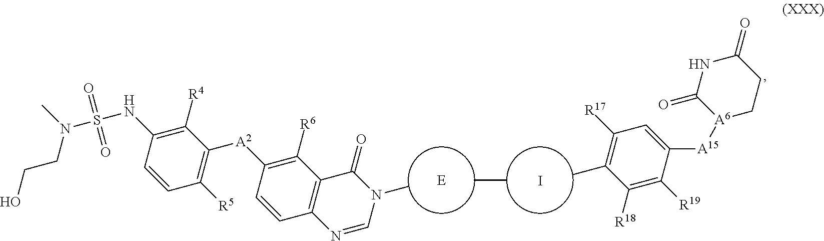

or a pharmaceutically acceptable salt thereof.

41. The compound of embodiment 1, wherein the compound is of Formula

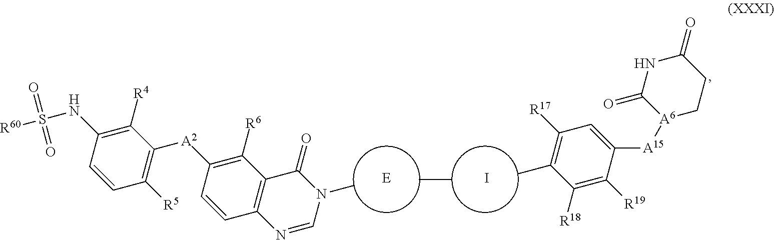

or a pharmaceutically acceptable salt thereof.

42. The compound of embodiment 1, wherein the compound is of Formula

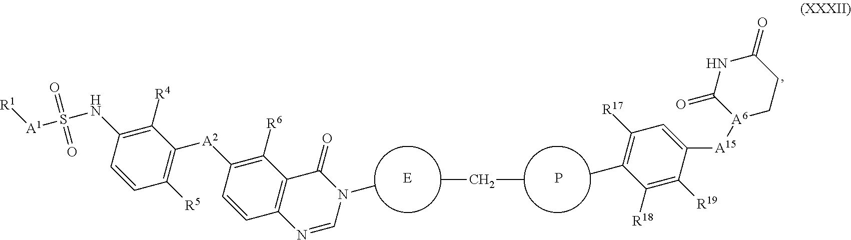

or a pharmaceutically acceptable salt thereof.

43. The compound of any one of embodiments 37-42, wherein Cycle \textcircled{E} is

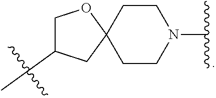 [SMILES: *C1COC2(CCN(*)CC2)C1]

44. The compound of any one of embodiments 37-42, wherein Cycle \textcircled{E} is

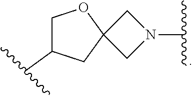 [SMILES: *C1COC2(C1)CN(*)C2]

45. The compound of embodiment 1, wherein the compound is of Formula

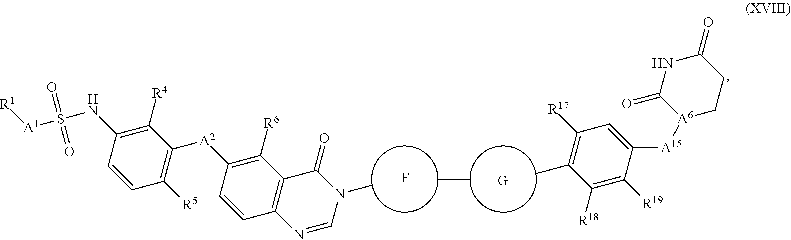

or a pharmaceutically acceptable salt thereof.

46. The compound of embodiment 1, wherein the compound is of Formula

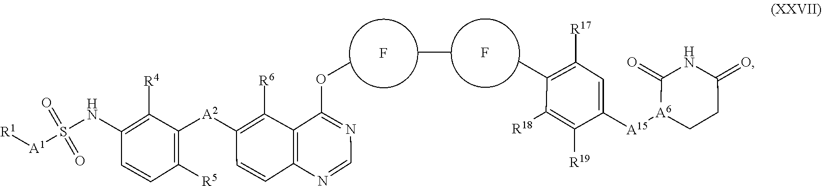

or a pharmaceutically acceptable salt thereof.

47. The compound of embodiment 45 or 46, wherein Cycle \textcircled{F} is

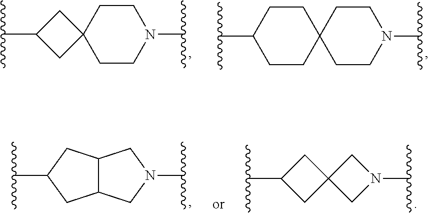

48. The compound of embodiment 45 or 46, wherein Cycle \textcircled{F} is

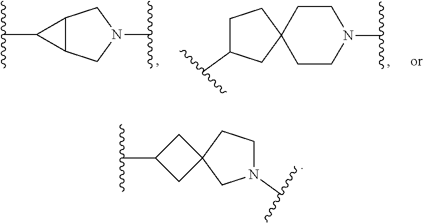 [SMILES: *C1C2CN(*)CC12 ; *C1CCC2(CCN(*)CC2)C1 ; *C1CC2(CCN(*)C2)C1]

49. The compound of embodiment 1, wherein the compound is of Formula

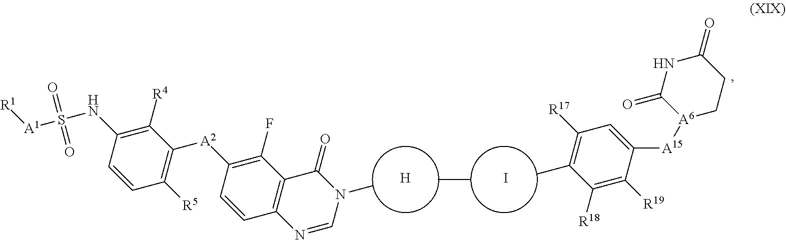

or a pharmaceutically acceptable salt thereof.

50. The compound of embodiment 1, wherein the compound is of Formula

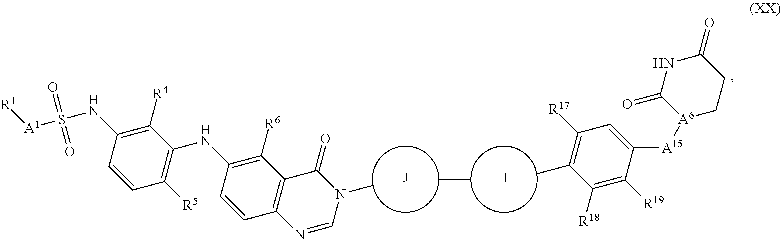

or a pharmaceutically acceptable salt thereof.

51. The compound of embodiment 1, wherein the compound is of Formula

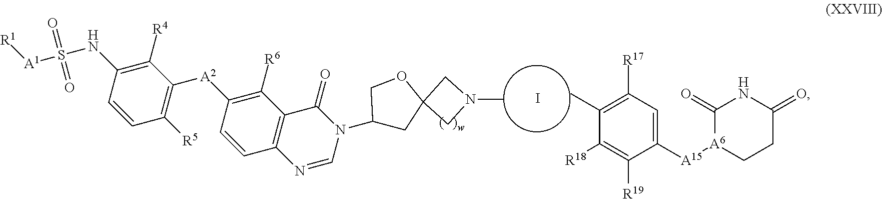

or a pharmaceutically acceptable salt thereof.

52. The compound of any one of embodiments 49-51, wherein Cycle \textcircled{I} is

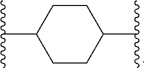 [SMILES: *C1CCC(*)CC1]

53. The compound of embodiment 1, wherein the compound is of Formula

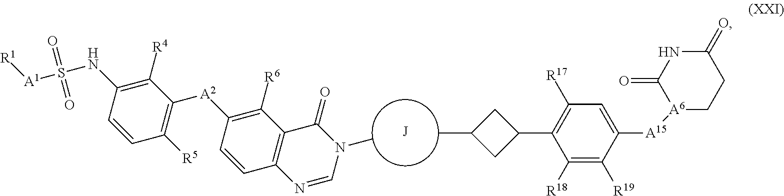

or a pharmaceutically acceptable salt thereof.

54. The compound of embodiment 1, wherein the compound is of Formula

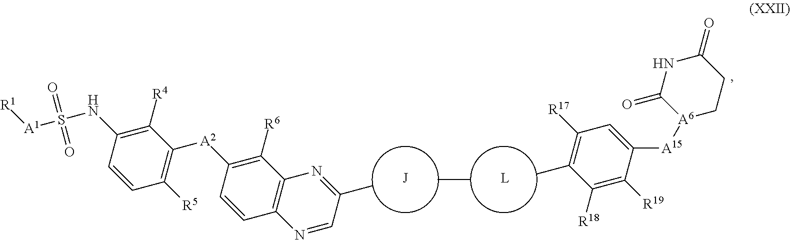

or a pharmaceutically acceptable salt thereof.

55. The compound of embodiment 1, wherein the compound is of Formula

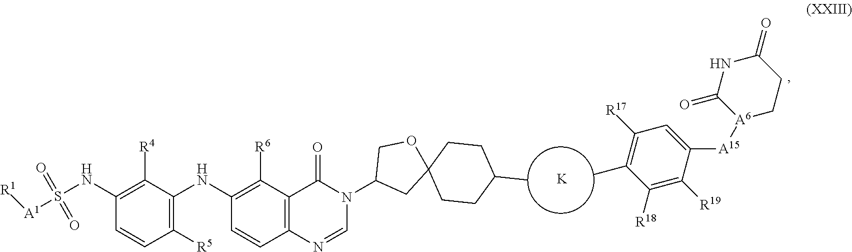

or a pharmaceutically acceptable salt thereof 56. The compound of embodiment 1, wherein the compound is of Formula

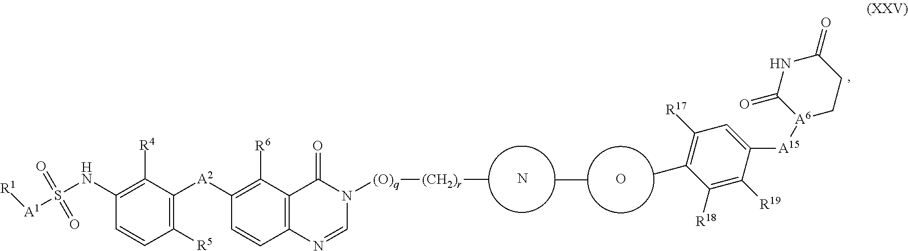

or a pharmaceutically acceptable salt thereof.

57. The compound of embodiment 1, wherein the compound is of Formula

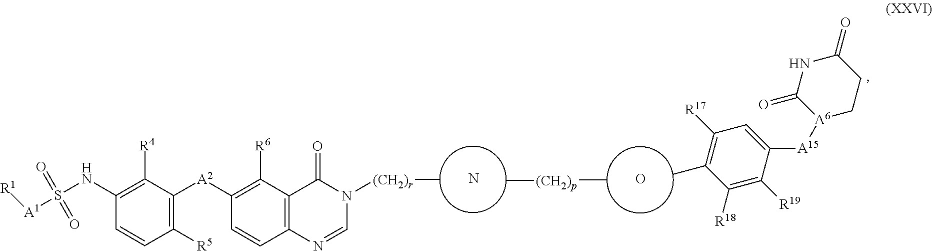

or a pharmaceutically acceptable salt thereof.

58. The compound of any one of embodiments 37-57 wherein A⁶ is CH and A¹⁵ is —N—.

59. The compound of any one of embodiments 37-57 wherein A⁶ is CH and A¹⁵ is —O—.

60. The compound of any one of embodiments 37-57 wherein A⁶ is CH and A¹⁵ is bond.

61. The compound of any one of embodiments 37-57 wherein A⁶ is N and A¹⁵ is bond.

62. The compound of any one of embodiments 37-61 wherein R¹⁷ is H.

63. The compound of any one of embodiments 37-61 wherein R¹⁷ is CH₃.

64. The compound of any one of embodiments 37-61 wherein R¹⁷ is cyano.

65. The compound of any one of embodiments 37-61 wherein R¹⁷ is hydroxy.

66. The compound of any one of embodiments 37-61 wherein R¹⁷ is cyclopropyl.

67. The compound of any one of embodiments 37-61 wherein R¹⁷ is F.

68. The compound of any one of embodiments 37-61 wherein R¹⁷ is OCH₃.

69. The compound of any one of embodiments 37-68 wherein R¹⁸ is H.

70. The compound of any one of embodiments 37-68 wherein R¹⁸ is CH₃.

71. The compound of any one of embodiments 37-68 wherein R¹⁸ is cyano.

72. The compound of any one of embodiments 37-68 wherein R¹⁸ is hydroxy.

73. The compound of any one of embodiments 37-68 wherein R¹⁸ is cyclopropyl.

74. The compound of any one of embodiments 37-68 wherein R¹⁸ is F.

75. The compound of any one of embodiments 37-68 wherein R¹⁸ is OCH₃.

76. The compound of any one of embodiments 37-75 wherein R¹⁹ is H.

77. The compound of any one of embodiments 37-75 wherein R¹⁹ is CH₃.

78. The compound of any one of embodiments 37-75 wherein R¹⁹ is cyano.

79. The compound of any one of embodiments 37-75 wherein R¹⁹ is hydroxy.

80. The compound of any one of embodiments 37-75 wherein R¹⁹ is cyclopropyl.

81. The compound of any one of embodiments 37-75 wherein R¹⁹ is F.

82. The compound of any one of embodiments 37-75 wherein R¹⁹ is OCH₃.

83. A compound described in Compound Table 1.

84. A pharmaceutical composition comprising a compound according to any one of embodiments 1-83, or a pharmaceutically acceptable salt thereof, and a pharmaceutically acceptable carrier.

85. A method of treating a mutant BRAF mediated disorder comprising administering an effective amount of a compound of any one of embodiments 1-83 or a pharmaceutically acceptable salt thereof or a pharmaceutical composition of embodiment 84 to a human patient in need thereof.

86. The method of embodiment 85, wherein the mutant BRAF mediated disorder is a cancer.

87. The method of embodiment 86, wherein the mutant BRAF mediated cancer is melanoma.

88. The method of embodiment 86, wherein the mutant BRAF mediated cancer is lung cancer.

89. The method of embodiment 86, wherein the mutant BRAF mediated cancer is non-small cell lung cancer.

90. The method of embodiment 86, wherein the mutant BRAF mediated cancer is colorectal cancer.

91. The method of embodiment 86, wherein the mutant BRAF mediated cancer is microsatellite stable colorectal cancer.

92. The method of embodiment 86, wherein the mutant BRAF mediated cancer is thyroid cancer.

93. The method of embodiment 86, wherein the mutant BRAF mediated cancer is ovarian cancer.

94. The method of embodiment 85 wherein the mutant BRAF mediated disorder is cholangiocarcinoma, erdeheim-chester disease, langerhans histiocytosis, ganglioglioma, glioma, glioblastoma, hairy cell leukemia, metanephric adenoma, multiple myeloma, non-small-cell lung cancer, ovarian cancer, pilomyxoid astrocytoma, anaplastic pleomorphic xanthoastrocytoma, astrocytoma, papillary thyroid cancer, anaplastic thyroid cancer, pancreatic cancer, thoracic clear cell sarcoma, salivary gland cancer, or microsatellite stable colorectal cancer.

95. The method of any one of embodiments 85-94, wherein the patient also receives an additional active agent.

96. The method of embodiment 95, wherein the additional active agent is a MEK inhibitor.

97. The method of embodiment 96, wherein the MEK inhibitor is trametinib.

98. The method of embodiment 95, wherein the additional active agent is an immune checkpoint inhibitor.

99. The method of embodiment 98, wherein the immune checkpoint inhibitor is selected from nivolumab, pembrolizumab, cemiplimab, ipilimumab, relatlimab, atezolizumab, avelumab, and durvalumab.

100. The method of embodiment 95, wherein the additional active agent is cetuximab or panitumumab.

101. The method of any one of embodiments 85-100, wherein the mutant BRAF has a V600 mutation.

102. The method of embodiment 101, wherein the V600 mutation is V600E, V600K, V600R, V600D, or V600N.

103. The method of embodiment 101, wherein the V600 mutation is V600E.

104. The method of any one of embodiments 85-103, wherein the mutant BRAF has a mutation selected from G469A, G469V, G469L, G469R, L597Q, and K601E.

105. The method of any one of embodiments 85-104, wherein the mutant BRAF has a mutation selected from G466A, G466E, G466R, G466V, S467L, G469E, N581I, D594E, D594G, and D594N.

106. The method of any one of embodiments 85-105, wherein the mutant BRAF has a mutation selected from G464I, G464R, N581T, L584F, E586K, G593D, G596C, L597R, L597S, S605I, S607F, N684T, E26A, V130M, L745L, and D284E.

107. The method of any one of embodiments 85-106, wherein the mutant BRAF has splice mutation.

108. The method of embodiment 107, wherein the splice mutation is a p61-BRAF splice mutation.

109. The method of any one of embodiments 85-108, wherein the mutant BRAF mediated disorder is a cancer and the cancer also has a NRAS, MEK1, or PI3K mutation.

110. The method of embodiment 109, wherein the cancer has a NRAS mutation.

111. The method of embodiment 110, wherein the NRAS mutation is Q61K.

112. The method of embodiment 110, wherein the NRAS mutation is Q61R.

113. The method of embodiment 109, wherein the cancer has a MEK1 mutation.

114. The method of embodiment 109, wherein the cancer has a PI3K mutation.

115. The method of embodiment 114, wherein PI3K mutation is PIK3CA^{H1047R} or PIK3CA^{P449T}.

116. The method of any one of embodiments 85-115, wherein the mutant BRAF mediated disorder is a cancer and wherein one or more mutations promote RAF protein homo-dimerization or hetero-dimerization.

117. The method of embodiment 116, wherein the one or more mutations promote RAF protein homo-dimerization.

118. The method of embodiment 116, wherein the one or more mutations promote RAF protein hetero-dimerization.

119. The method of any one of embodiments 85-118, wherein the mutant BRAF mediated disorder is a cancer that has metastasized to the brain or CNS.

120. The method of any one of embodiments 85-118, wherein the mutant BRAF mediated disorder is a cancer that has metastasized to the brain.

121. The method of any one of embodiments 85-118, wherein the mutant BRAF mediated disorder is a cancer that has metastasized to the CNS.

### Non-Limiting Examples of Compounds of the Present Invention

COMPOUND TABLE 1
Structure

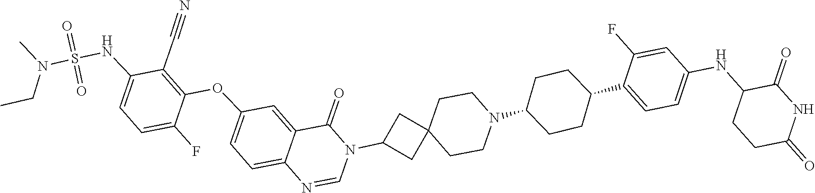 [SMILES: CCN(C)S(=O)(=O)Nc1ccc(F)c(Oc2ccc3ncn(C4CC5(CCN([C@H]6CC[C@@H](c7ccc(NC8CCC(=O)NC8=O)cc7F)CC6)CC5)C4)c(=O)c3c2)c1C#N]

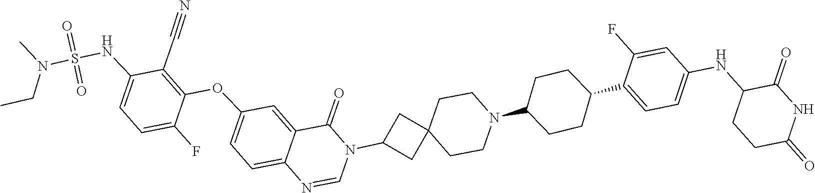

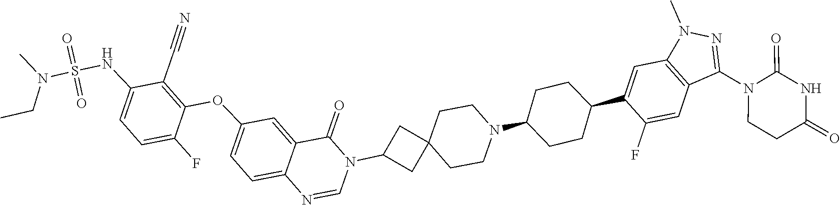 [SMILES: CCN(C)S(=O)(=O)Nc1ccc(F)c(Oc2ccc3ncn(C4CC5(CCN([C@H]6CC[C@@H](c7cc8c(cc7F)c(N7CCC(=O)NC7=O)nn8C)CC6)CC5)C4)c(=O)c3c2)c1C#N]

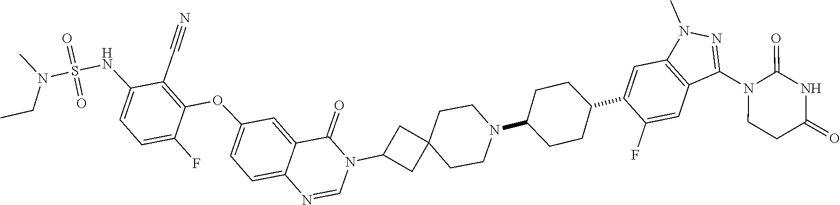

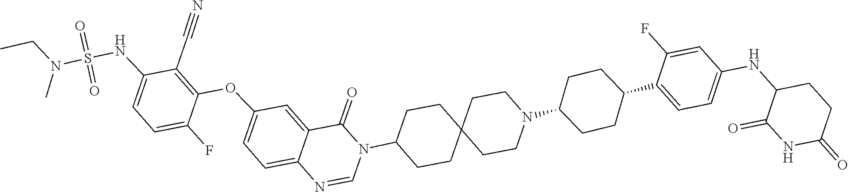 [SMILES: CCN(C)S(=O)(=O)Nc1ccc(F)c(Oc2ccc3ncn(C4CCC5(CC4)CCN([C@H]4CC[C@@H](c6ccc(NC7CCC(=O)NC7=O)cc6F)CC4)CC5)c(=O)c3c2)c1C#N]

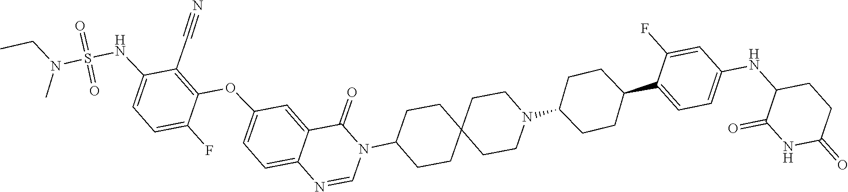 [SMILES: CCN(C)S(=O)(=O)Nc1ccc(F)c(Oc2ccc3ncn(C4CCC5(CC4)CCN([C@H]4CC[C@H](c6ccc(NC7CCC(=O)NC7=O)cc6F)CC4)CC5)c(=O)c3c2)c1C#N]

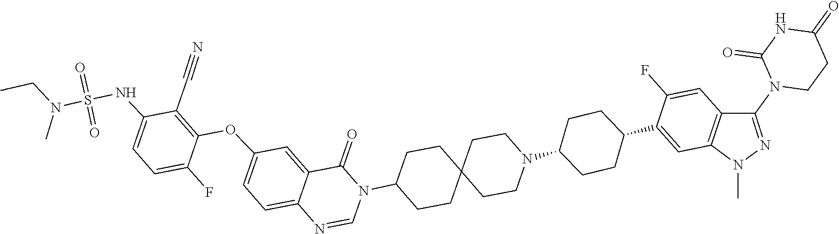 [SMILES: CCN(C)S(=O)(=O)Nc1ccc(F)c(Oc2ccc3ncn(C4CCC5(CC4)CCN([C@H]4CC[C@@H](c6cc7c(cc6F)c(N6CCC(=O)NC6=O)nn7C)CC4)CC5)c(=O)c3c2)c1C#N]

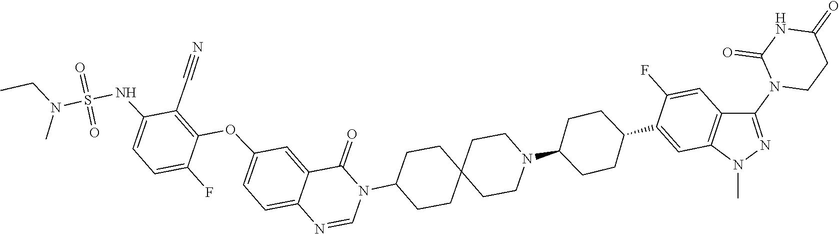 [SMILES: CCN(C)S(=O)(=O)Nc1ccc(F)c(Oc2ccc3ncn(C4CCC5(CC4)CCN([C@H]4CC[C@H](c6cc7c(cc6F)c(N6CCC(=O)NC6=O)nn7C)CC4)CC5)c(=O)c3c2)c1C#N]

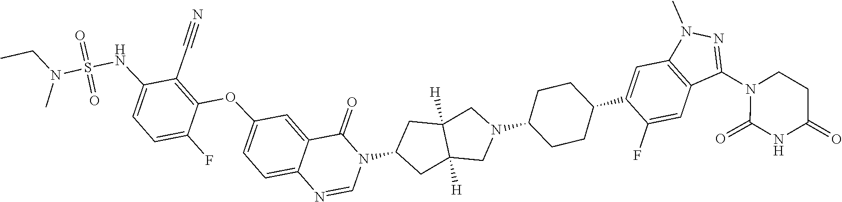 [SMILES: CCN(C)S(=O)(=O)Nc1ccc(F)c(Oc2ccc3ncn([C@H]4C[C@@H]5CN([C@H]6CC[C@@H](c7cc8c(cc7F)c(N7CCC(=O)NC7=O)nn8C)CC6)C[C@@H]5C4)c(=O)c3c2)c1C#N]

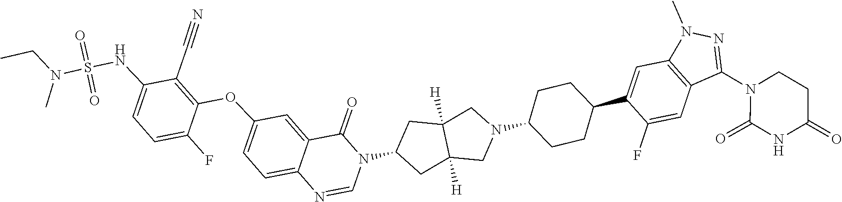 [SMILES: CCN(C)S(=O)(=O)Nc1ccc(F)c(Oc2ccc3ncn([C@H]4C[C@@H]5CN([C@H]6CC[C@H](c7cc8c(cc7F)c(N7CCC(=O)NC7=O)nn8C)CC6)C[C@@H]5C4)c(=O)c3c2)c1C#N]

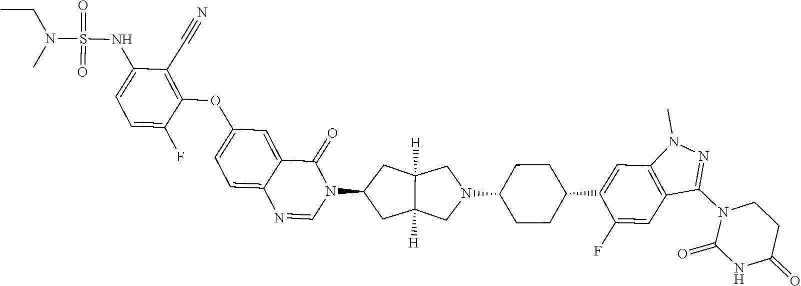 [SMILES: CCN(C)S(=O)(=O)Nc1ccc(F)c(Oc2ccc3ncn([C@@H]4C[C@@H]5CN([C@H]6CC[C@@H](c7cc8c(cc7F)c(N7CCC(=O)NC7=O)nn8C)CC6)C[C@@H]5C4)c(=O)c3c2)c1C#N]

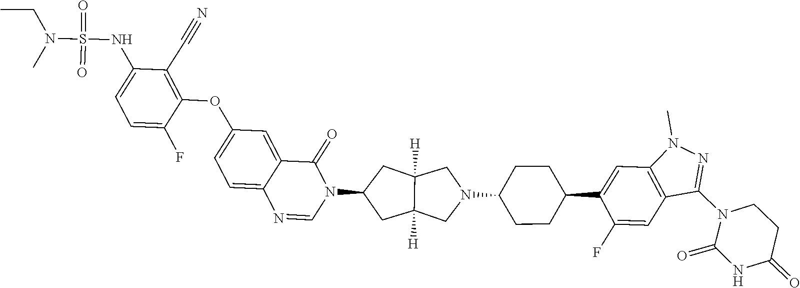 [SMILES: CCN(C)S(=O)(=O)Nc1ccc(F)c(Oc2ccc3ncn([C@@H]4C[C@@H]5CN([C@H]6CC[C@H](c7cc8c(cc7F)c(N7CCC(=O)NC7=O)nn8C)CC6)C[C@@H]5C4)c(=O)c3c2)c1C#N]

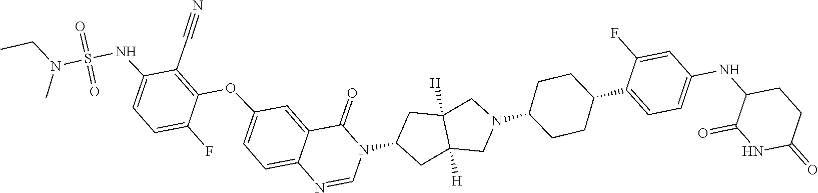 [SMILES: CCN(C)S(=O)(=O)Nc1ccc(F)c(Oc2ccc3ncn([C@H]4C[C@@H]5CN([C@H]6CC[C@@H](c7ccc(NC8CCC(=O)NC8=O)cc7F)CC6)C[C@@H]5C4)c(=O)c3c2)c1C#N]

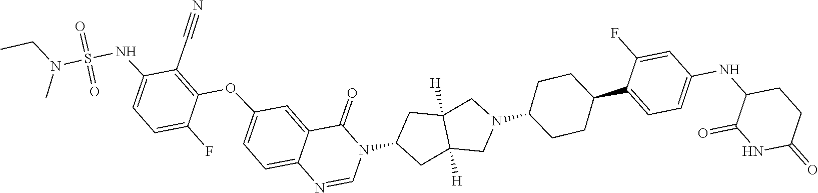 [SMILES: CCN(C)S(=O)(=O)Nc1ccc(F)c(Oc2ccc3ncn([C@H]4C[C@@H]5CN([C@H]6CC[C@H](c7ccc(NC8CCC(=O)NC8=O)cc7F)CC6)C[C@@H]5C4)c(=O)c3c2)c1C#N]

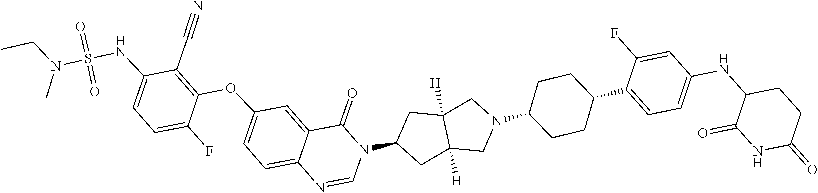 [SMILES: CCN(C)S(=O)(=O)Nc1ccc(F)c(Oc2ccc3ncn([C@@H]4C[C@@H]5CN([C@H]6CC[C@@H](c7ccc(NC8CCC(=O)NC8=O)cc7F)CC6)C[C@@H]5C4)c(=O)c3c2)c1C#N]

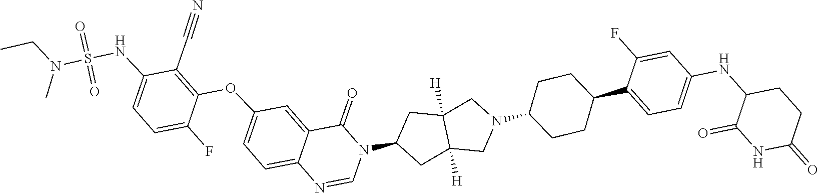 [SMILES: CCN(C)S(=O)(=O)Nc1ccc(F)c(Oc2ccc3ncn([C@@H]4C[C@@H]5CN([C@H]6CC[C@H](c7ccc(NC8CCC(=O)NC8=O)cc7F)CC6)C[C@@H]5C4)c(=O)c3c2)c1C#N]

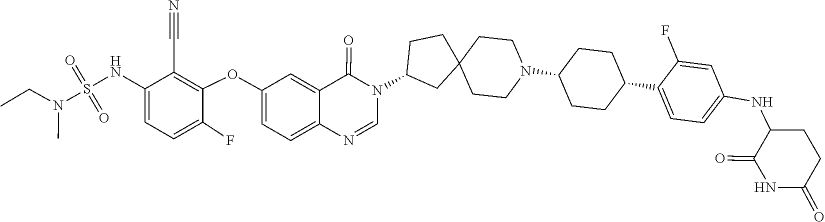 [SMILES: CCN(C)S(=O)(=O)Nc1ccc(F)c(Oc2ccc3ncn([C@@H]4CCC5(CCN([C@H]6CC[C@@H](c7ccc(NC8CCC(=O)NC8=O)cc7F)CC6)CC5)C4)c(=O)c3c2)c1C#N]

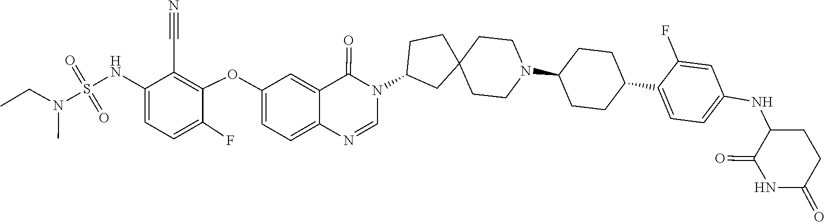 [SMILES: CCN(C)S(=O)(=O)Nc1ccc(F)c(Oc2ccc3ncn([C@@H]4CCC5(CCN([C@H]6CC[C@H](c7ccc(NC8CCC(=O)NC8=O)cc7F)CC6)CC5)C4)c(=O)c3c2)c1C#N]

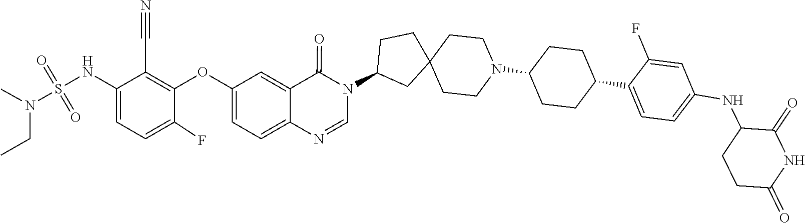 [SMILES: CCN(C)S(=O)(=O)Nc1ccc(F)c(Oc2ccc3ncn([C@H]4CCC5(CCN([C@H]6CC[C@@H](c7ccc(NC8CCC(=O)NC8=O)cc7F)CC6)CC5)C4)c(=O)c3c2)c1C#N]

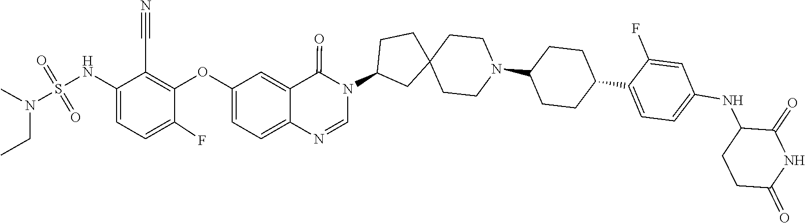

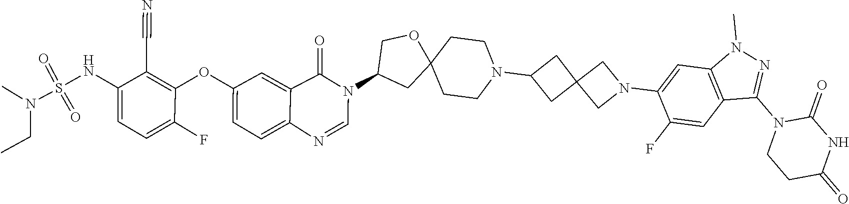 [SMILES: CCN(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N]

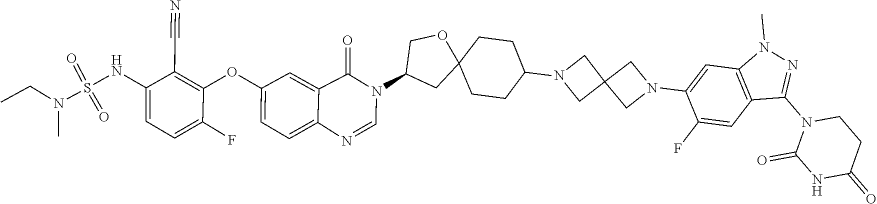 [SMILES: CCN(C)S(=O)(=O)Nc1ccc(F)c(Oc2ccc3ncn([C@H]4COC5(CCC(N6CC7(CN(c8cc9c(cc8F)c(N8CCC(=O)NC8=O)nn9C)C7)C6)CC5)C4)c(=O)c3c2)c1C#N]

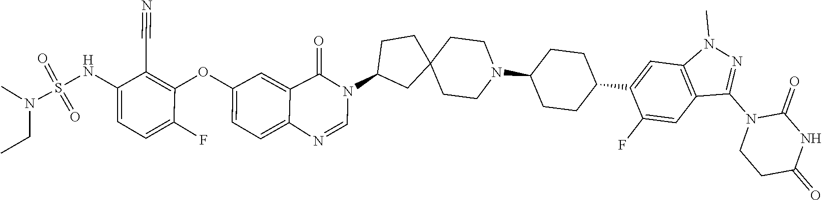 [SMILES: CCN(C)S(=O)(=O)Nc1ccc(F)c(Oc2ccc3ncn([C@H]4CCC5(CCN([C@H]6CC[C@H](c7cc8c(cc7F)c(N7CCC(=O)NC7=O)nn8C)CC6)CC5)C4)c(=O)c3c2)c1C#N]

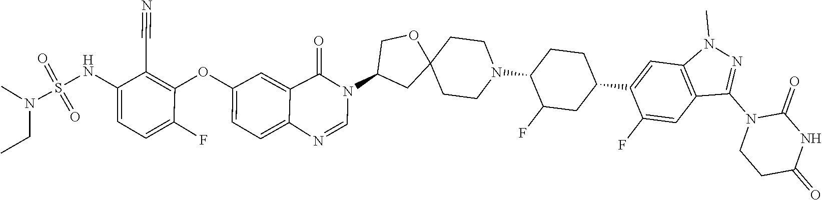 [SMILES: CCN(C)S(=O)(=O)Nc1ccc(F)c(Oc2ccc3ncn([C@H]4COC5(CCN([C@@H]6CC[C@H](c7cc8c(cc7F)c(N7CCC(=O)NC7=O)nn8C)CC6F)CC5)C4)c(=O)c3c2)c1C#N]

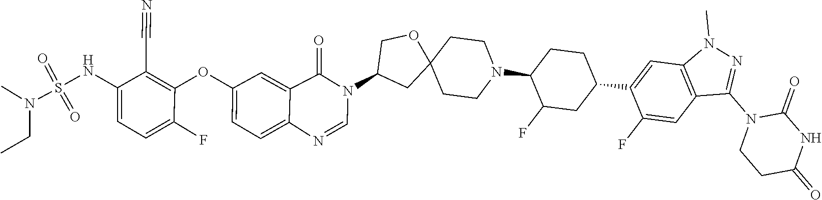 [SMILES: CCN(C)S(=O)(=O)Nc1ccc(F)c(Oc2ccc3ncn([C@H]4COC5(CCN([C@H]6CC[C@H](c7cc8c(cc7F)c(N7CCC(=O)NC7=O)nn8C)CC6F)CC5)C4)c(=O)c3c2)c1C#N]

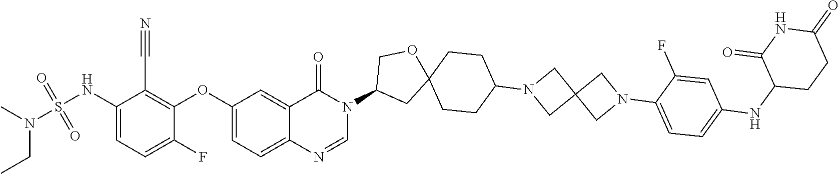 [SMILES: CCN(C)S(=O)(=O)Nc1ccc(F)c(Oc2ccc3ncn([C@H]4COC5(CCC(N6CC7(CN(c8ccc(NC9CCC(=O)NC9=O)cc8F)C7)C6)CC5)C4)c(=O)c3c2)c1C#N]

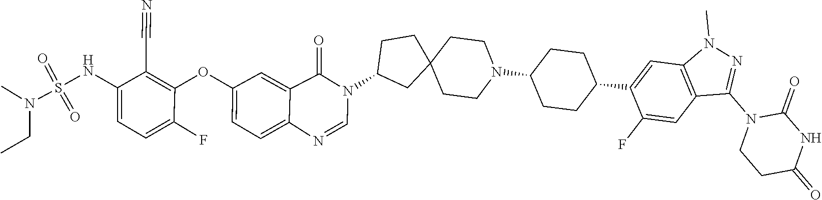 [SMILES: CCN(C)S(=O)(=O)Nc1ccc(F)c(Oc2ccc3ncn([C@@H]4CCC5(CCN([C@H]6CC[C@@H](c7cc8c(cc7F)c(N7CCC(=O)NC7=O)nn8C)CC6)CC5)C4)c(=O)c3c2)c1C#N]

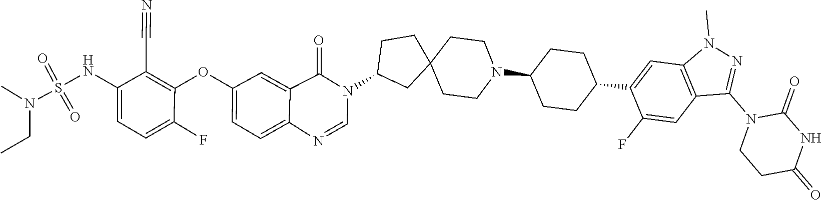 [SMILES: CCN(C)S(=O)(=O)Nc1ccc(F)c(Oc2ccc3ncn([C@@H]4CCC5(CCN([C@H]6CC[C@H](c7cc8c(cc7F)c(N7CCC(=O)NC7=O)nn8C)CC6)CC5)C4)c(=O)c3c2)c1C#N]

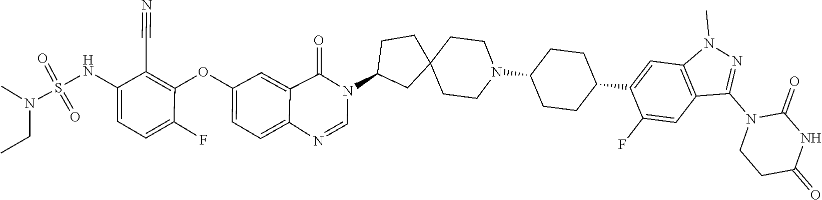 [SMILES: CCN(C)S(=O)(=O)Nc1ccc(F)c(Oc2ccc3ncn([C@H]4CCC5(CCN([C@H]6CC[C@@H](c7cc8c(cc7F)c(N7CCC(=O)NC7=O)nn8C)CC6)CC5)C4)c(=O)c3c2)c1C#N]

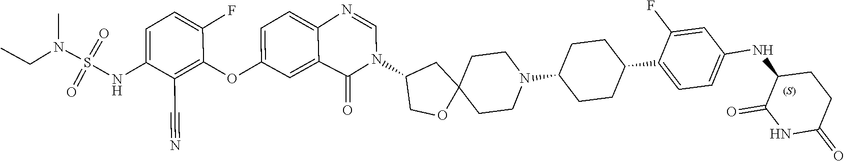

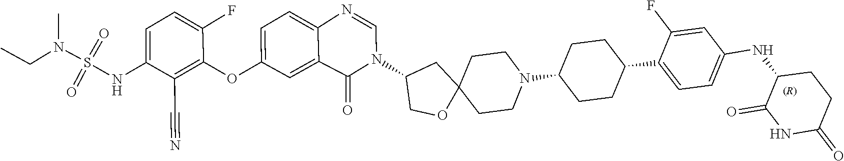

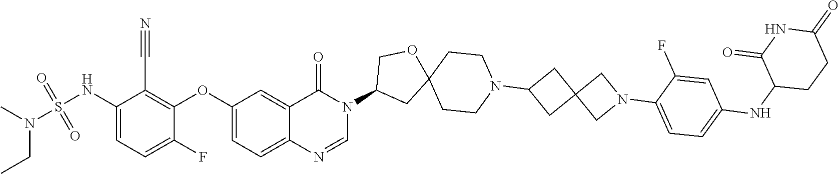 [SMILES: CCN(C)S(=O)(=O)Nc1ccc(F)c(Oc2ccc3ncn([C@H]4COC5(CCN(C6CC7(C6)CN(c6ccc(NC8CCC(=O)NC8=O)cc6F)C7)CC5)C4)c(=O)c3c2)c1C#N]

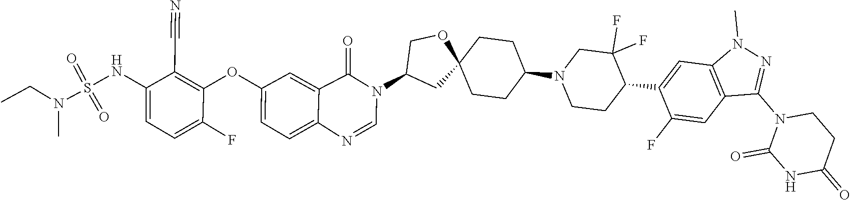 [SMILES: CCN(C)S(=O)(=O)Nc1ccc(F)c(Oc2ccc3ncn([C@H]4CO[C@]5(CC[C@@H](N6CC[C@@H](c7cc8c(cc7F)c(N7CCC(=O)NC7=O)nn8C)C(F)(F)C6)CC5)C4)c(=O)c3c2)c1C#N]

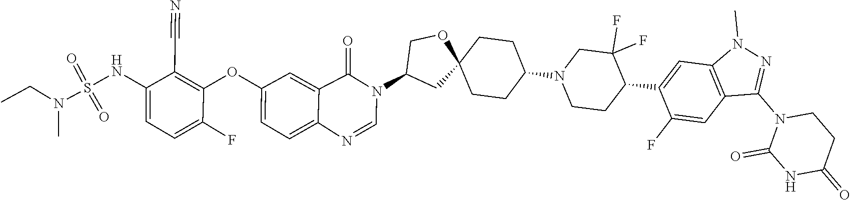 [SMILES: CCN(C)S(=O)(=O)Nc1ccc(F)c(Oc2ccc3ncn([C@H]4CO[C@]5(CC[C@H](N6CC[C@@H](c7cc8c(cc7F)c(N7CCC(=O)NC7=O)nn8C)C(F)(F)C6)CC5)C4)c(=O)c3c2)c1C#N]

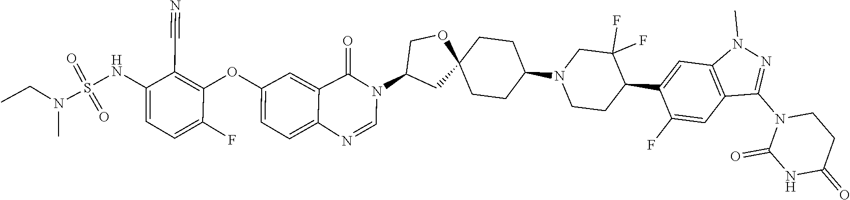 [SMILES: CCN(C)S(=O)(=O)Nc1ccc(F)c(Oc2ccc3ncn([C@H]4CO[C@]5(CC[C@@H](N6CC[C@H](c7cc8c(cc7F)c(N7CCC(=O)NC7=O)nn8C)C(F)(F)C6)CC5)C4)c(=O)c3c2)c1C#N]

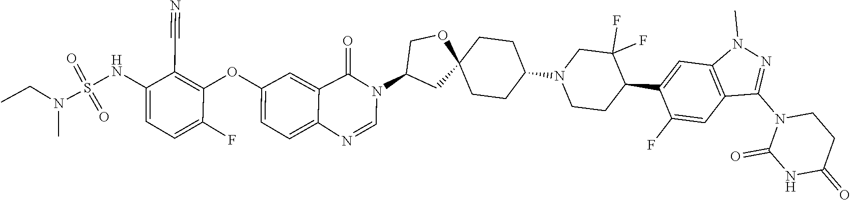 [SMILES: CCN(C)S(=O)(=O)Nc1ccc(F)c(Oc2ccc3ncn([C@H]4CO[C@]5(CC[C@H](N6CC[C@H](c7cc8c(cc7F)c(N7CCC(=O)NC7=O)nn8C)C(F)(F)C6)CC5)C4)c(=O)c3c2)c1C#N]

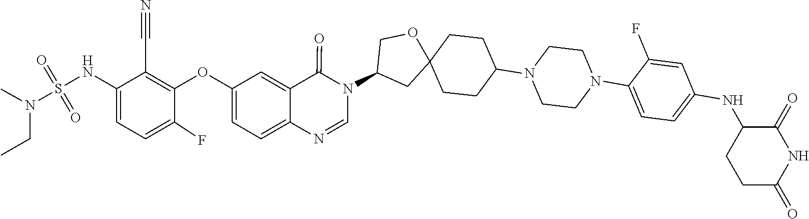 [SMILES: CCN(C)S(=O)(=O)Nc1ccc(F)c(Oc2ccc3ncn([C@H]4COC5(CCC(N6CCN(c7ccc(NC8CCC(=O)NC8=O)cc7F)CC6)CC5)C4)c(=O)c3c2)c1C#N]

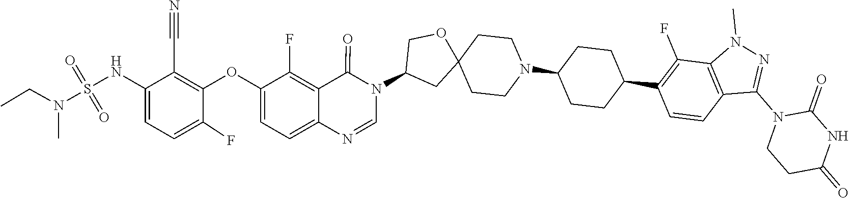 [SMILES: CCN(C)S(=O)(=O)Nc1ccc(F)c(Oc2ccc3ncn([C@H]4COC5(CCN([C@H]6CC[C@@H](c7ccc8c(N9CCC(=O)NC9=O)nn(C)c8c7F)CC6)CC5)C4)c(=O)c3c2F)c1C#N]

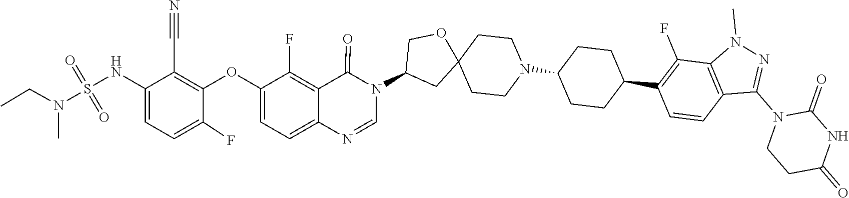 [SMILES: CCN(C)S(=O)(=O)Nc1ccc(F)c(Oc2ccc3ncn([C@H]4COC5(CCN([C@H]6CC[C@H](c7ccc8c(N9CCC(=O)NC9=O)nn(C)c8c7F)CC6)CC5)C4)c(=O)c3c2F)c1C#N]

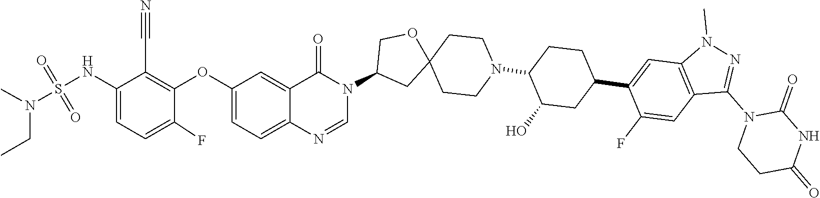

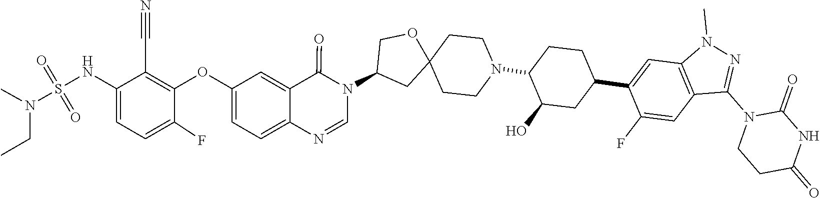

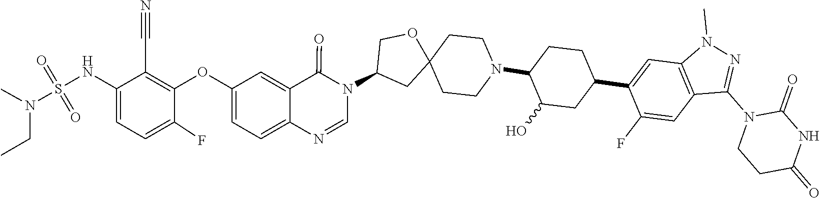

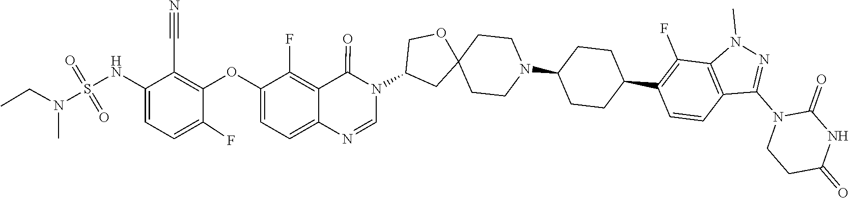 [SMILES: CCN(C)S(=O)(=O)Nc1ccc(F)c(Oc2ccc3ncn([C@@H]4COC5(CCN([C@H]6CC[C@@H](c7ccc8c(N9CCC(=O)NC9=O)nn(C)c8c7F)CC6)CC5)C4)c(=O)c3c2F)c1C#N]

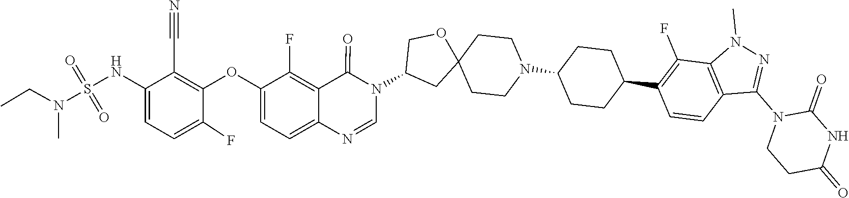 [SMILES: CCN(C)S(=O)(=O)Nc1ccc(F)c(Oc2ccc3ncn([C@@H]4COC5(CCN([C@H]6CC[C@H](c7ccc8c(N9CCC(=O)NC9=O)nn(C)c8c7F)CC6)CC5)C4)c(=O)c3c2F)c1C#N]

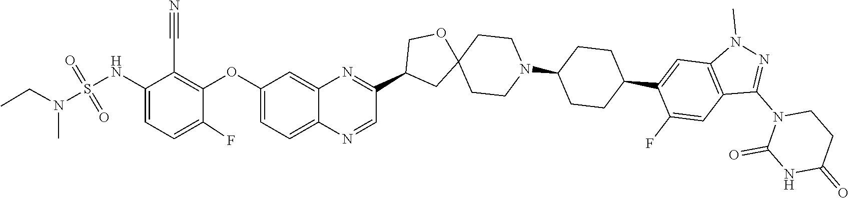 [SMILES: CCN(C)S(=O)(=O)Nc1ccc(F)c(Oc2ccc3ncc([C@H]4COC5(CCN([C@H]6CC[C@@H](c7cc8c(cc7F)c(N7CCC(=O)NC7=O)nn8C)CC6)CC5)C4)nc3c2)c1C#N]

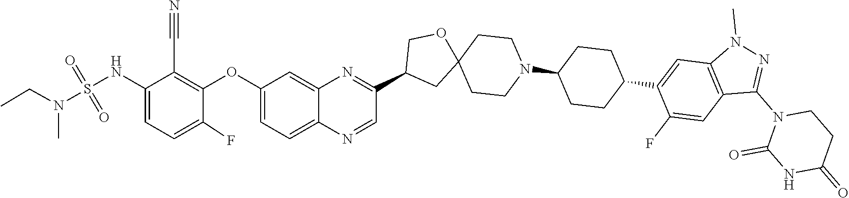 [SMILES: CCN(C)S(=O)(=O)Nc1ccc(F)c(Oc2ccc3ncc([C@H]4COC5(CCN([C@H]6CC[C@H](c7cc8c(cc7F)c(N7CCC(=O)NC7=O)nn8C)CC6)CC5)C4)nc3c2)c1C#N]

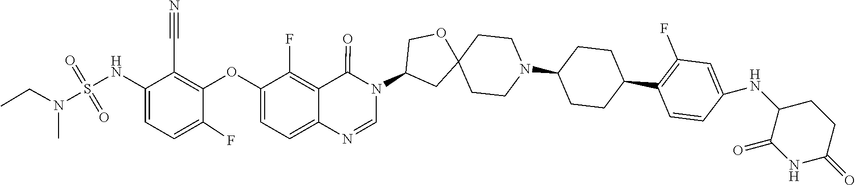 [SMILES: CCN(C)S(=O)(=O)Nc1ccc(F)c(Oc2ccc3ncn([C@H]4COC5(CCN([C@H]6CC[C@@H](c7ccc(NC8CCC(=O)NC8=O)cc7F)CC6)CC5)C4)c(=O)c3c2F)c1C#N]

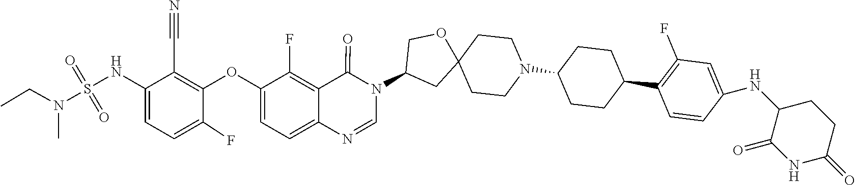 [SMILES: CCN(C)S(=O)(=O)Nc1ccc(F)c(Oc2ccc3ncn([C@H]4COC5(CCN([C@H]6CC[C@H](c7ccc(NC8CCC(=O)NC8=O)cc7F)CC6)CC5)C4)c(=O)c3c2F)c1C#N]

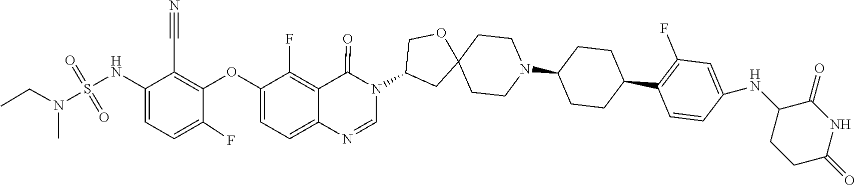 [SMILES: CCN(C)S(=O)(=O)Nc1ccc(F)c(Oc2ccc3ncn([C@@H]4COC5(CCN([C@H]6CC[C@@H](c7ccc(NC8CCC(=O)NC8=O)cc7F)CC6)CC5)C4)c(=O)c3c2F)c1C#N]

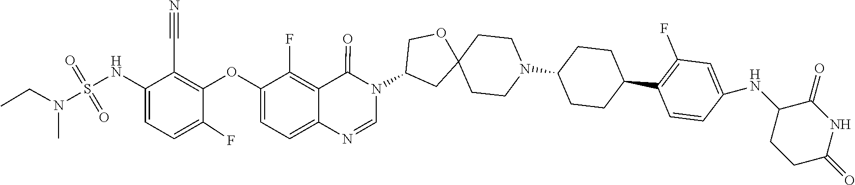 [SMILES: CCN(C)S(=O)(=O)Nc1ccc(F)c(Oc2ccc3ncn([C@@H]4COC5(CCN([C@H]6CC[C@H](c7ccc(NC8CCC(=O)NC8=O)cc7F)CC6)CC5)C4)c(=O)c3c2F)c1C#N]

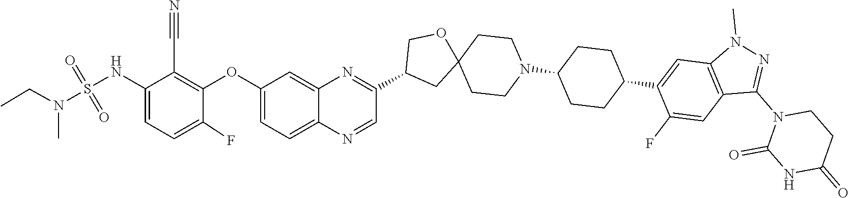 [SMILES: CCN(C)S(=O)(=O)Nc1ccc(F)c(Oc2ccc3ncc([C@@H]4COC5(CCN([C@H]6CC[C@@H](c7cc8c(cc7F)c(N7CCC(=O)NC7=O)nn8C)CC6)CC5)C4)nc3c2)c1C#N]

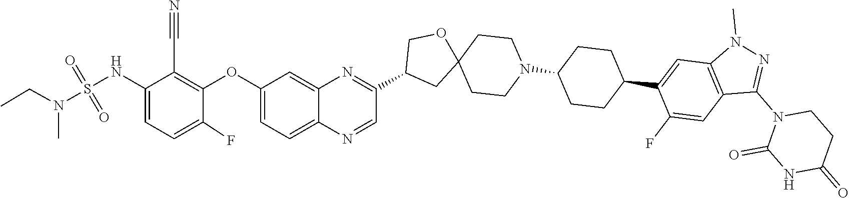 [SMILES: CCN(C)S(=O)(=O)Nc1ccc(F)c(Oc2ccc3ncc([C@@H]4COC5(CCN([C@H]6CC[C@H](c7cc8c(cc7F)c(N7CCC(=O)NC7=O)nn8C)CC6)CC5)C4)nc3c2)c1C#N]

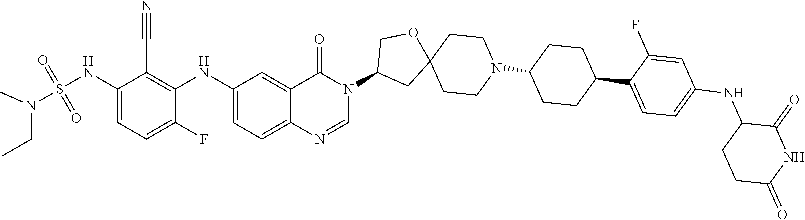 [SMILES: CCN(C)S(=O)(=O)Nc1ccc(F)c(Nc2ccc3ncn([C@H]4COC5(CCN([C@H]6CC[C@H](c7ccc(NC8CCC(=O)NC8=O)cc7F)CC6)CC5)C4)c(=O)c3c2)c1C#N]

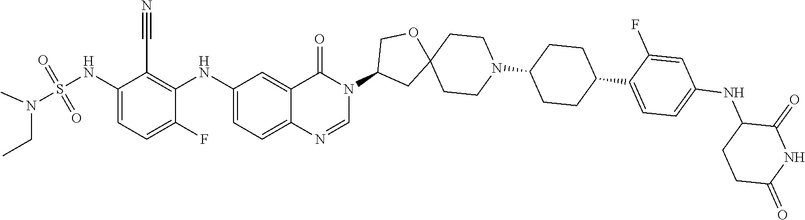 [SMILES: CCN(C)S(=O)(=O)Nc1ccc(F)c(Nc2ccc3ncn([C@H]4COC5(CCN([C@H]6CC[C@@H](c7ccc(NC8CCC(=O)NC8=O)cc7F)CC6)CC5)C4)c(=O)c3c2)c1C#N]

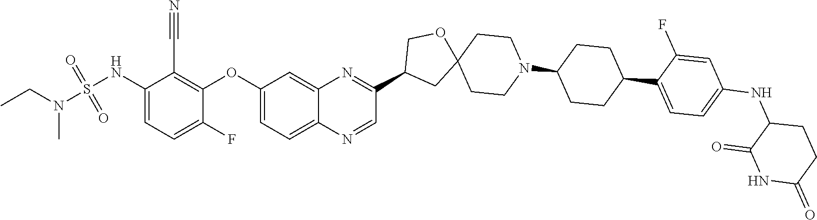 [SMILES: CCN(C)S(=O)(=O)Nc1ccc(F)c(Oc2ccc3ncc([C@H]4COC5(CCN([C@H]6CC[C@@H](c7ccc(NC8CCC(=O)NC8=O)cc7F)CC6)CC5)C4)nc3c2)c1C#N]

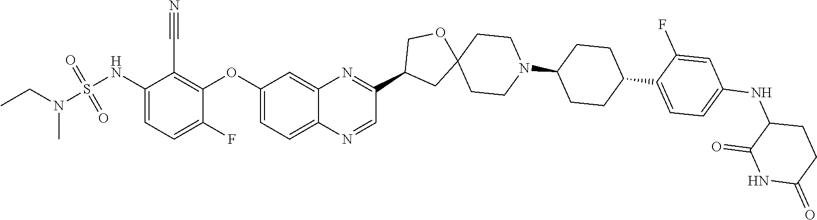 [SMILES: CCN(C)S(=O)(=O)Nc1ccc(F)c(Oc2ccc3ncc([C@H]4COC5(CCN([C@H]6CC[C@H](c7ccc(NC8CCC(=O)NC8=O)cc7F)CC6)CC5)C4)nc3c2)c1C#N]

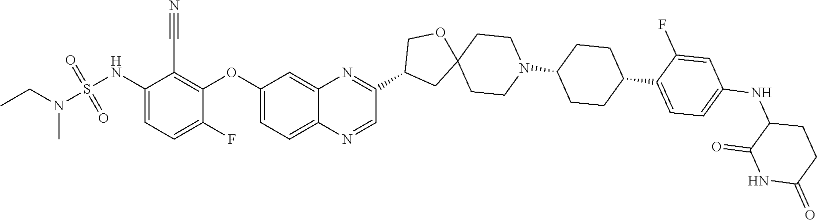 [SMILES: CCN(C)S(=O)(=O)Nc1ccc(F)c(Oc2ccc3ncc([C@@H]4COC5(CCN([C@H]6CC[C@@H](c7ccc(NC8CCC(=O)NC8=O)cc7F)CC6)CC5)C4)nc3c2)c1C#N]

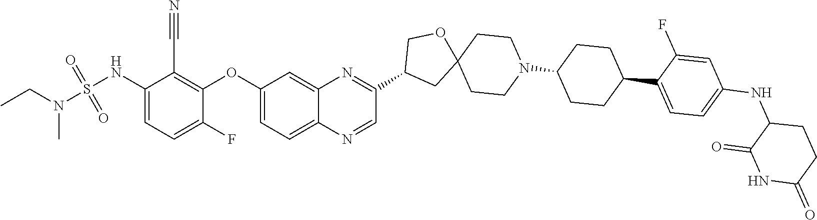 [SMILES: CCN(C)S(=O)(=O)Nc1ccc(F)c(Oc2ccc3ncc([C@@H]4COC5(CCN([C@H]6CC[C@H](c7ccc(NC8CCC(=O)NC8=O)cc7F)CC6)CC5)C4)nc3c2)c1C#N]

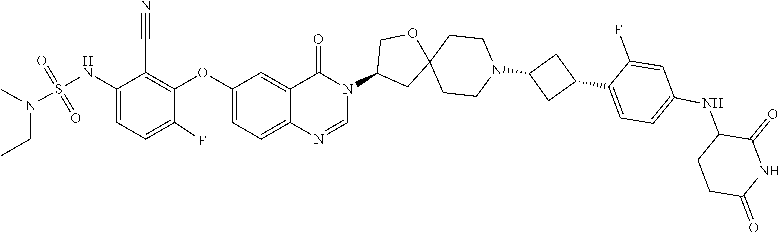 [SMILES: CCN(C)S(=O)(=O)Nc1ccc(F)c(Oc2ccc3ncn([C@H]4COC5(CCN([C@H]6C[C@@H](c7ccc(NC8CCC(=O)NC8=O)cc7F)C6)CC5)C4)c(=O)c3c2)c1C#N]

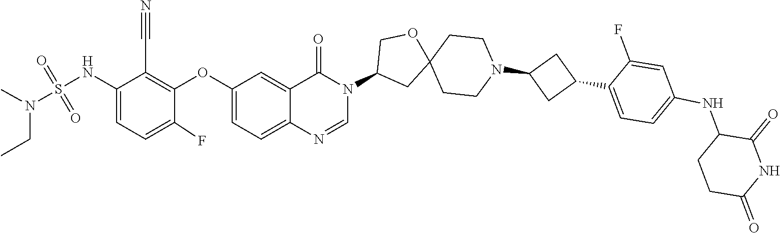 [SMILES: CCN(C)S(=O)(=O)Nc1ccc(F)c(Oc2ccc3ncn([C@H]4COC5(CCN([C@H]6C[C@H](c7ccc(NC8CCC(=O)NC8=O)cc7F)C6)CC5)C4)c(=O)c3c2)c1C#N]

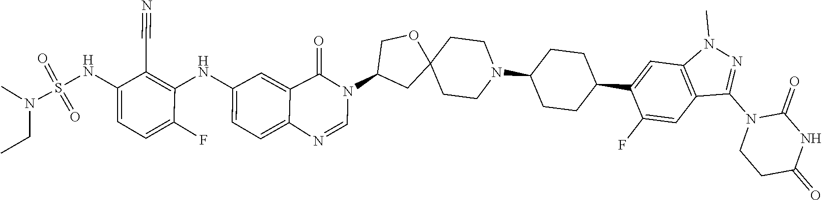 [SMILES: CCN(C)S(=O)(=O)Nc1ccc(F)c(Nc2ccc3ncn([C@H]4COC5(CCN([C@H]6CC[C@@H](c7cc8c(cc7F)c(N7CCC(=O)NC7=O)nn8C)CC6)CC5)C4)c(=O)c3c2)c1C#N]

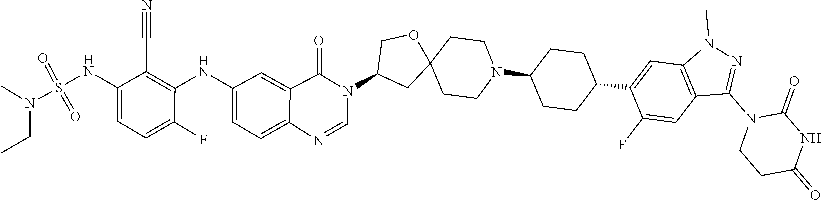 [SMILES: CCN(C)S(=O)(=O)Nc1ccc(F)c(Nc2ccc3ncn([C@H]4COC5(CCN([C@H]6CC[C@H](c7cc8c(cc7F)c(N7CCC(=O)NC7=O)nn8C)CC6)CC5)C4)c(=O)c3c2)c1C#N]

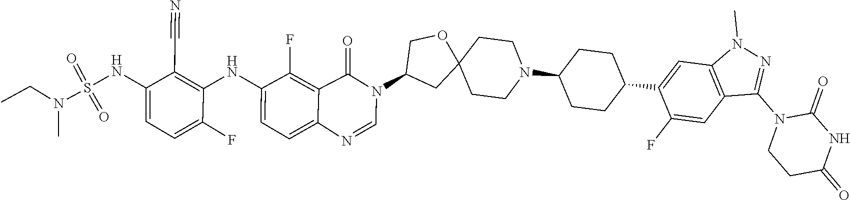 [SMILES: CCN(C)S(=O)(=O)Nc1ccc(F)c(Nc2ccc3ncn([C@H]4COC5(CCN([C@H]6CC[C@H](c7cc8c(cc7F)c(N7CCC(=O)NC7=O)nn8C)CC6)CC5)C4)c(=O)c3c2F)c1C#N]

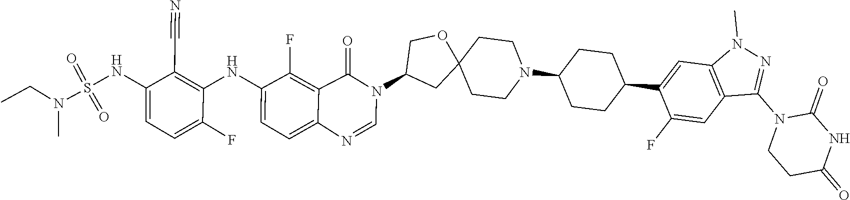 [SMILES: CCN(C)S(=O)(=O)Nc1ccc(F)c(Nc2ccc3ncn([C@H]4COC5(CCN([C@H]6CC[C@@H](c7cc8c(cc7F)c(N7CCC(=O)NC7=O)nn8C)CC6)CC5)C4)c(=O)c3c2F)c1C#N]

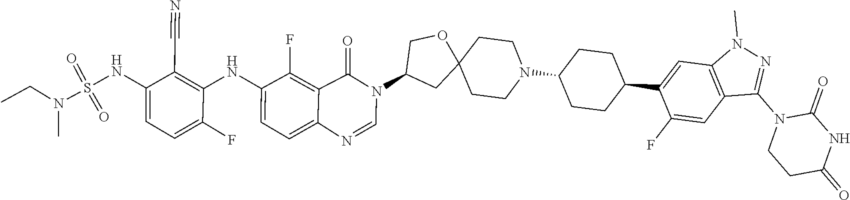 [SMILES: CCN(C)S(=O)(=O)Nc1ccc(F)c(Nc2ccc3ncn([C@H]4COC5(CCN([C@H]6CC[C@H](c7cc8c(cc7F)c(N7CCC(=O)NC7=O)nn8C)CC6)CC5)C4)c(=O)c3c2F)c1C#N]

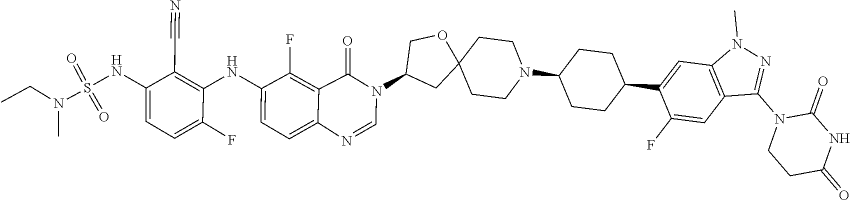 [SMILES: CCN(C)S(=O)(=O)Nc1ccc(F)c(Nc2ccc3ncn([C@H]4COC5(CCN([C@H]6CC[C@@H](c7cc8c(cc7F)c(N7CCC(=O)NC7=O)nn8C)CC6)CC5)C4)c(=O)c3c2F)c1C#N]

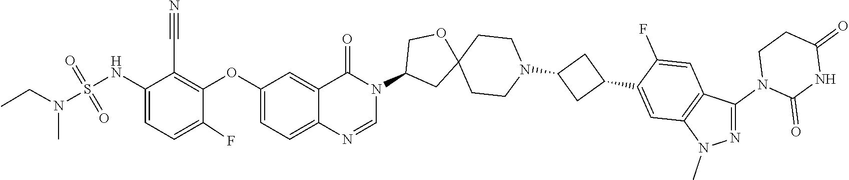 [SMILES: CCN(C)S(=O)(=O)Nc1ccc(F)c(Oc2ccc3ncn([C@H]4COC5(CCN([C@H]6C[C@@H](c7cc8c(cc7F)c(N7CCC(=O)NC7=O)nn8C)C6)CC5)C4)c(=O)c3c2)c1C#N]

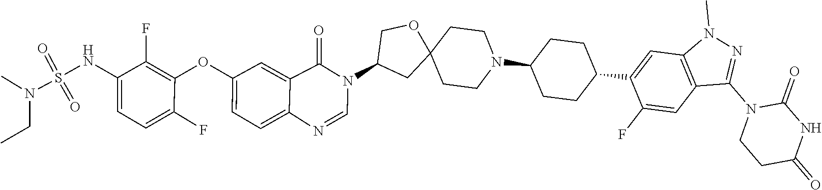 [SMILES: CCN(C)S(=O)(=O)Nc1ccc(F)c(Oc2ccc3ncn([C@H]4COC5(CCN([C@H]6CC[C@H](c7cc8c(cc7F)c(N7CCC(=O)NC7=O)nn8C)CC6)CC5)C4)c(=O)c3c2)c1F]

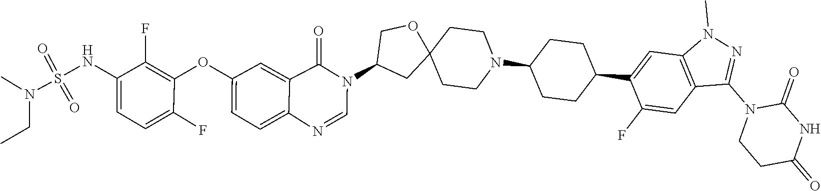 [SMILES: CCN(C)S(=O)(=O)Nc1ccc(F)c(Oc2ccc3ncn([C@H]4COC5(CCN([C@H]6CC[C@@H](c7cc8c(cc7F)c(N7CCC(=O)NC7=O)nn8C)CC6)CC5)C4)c(=O)c3c2)c1F]

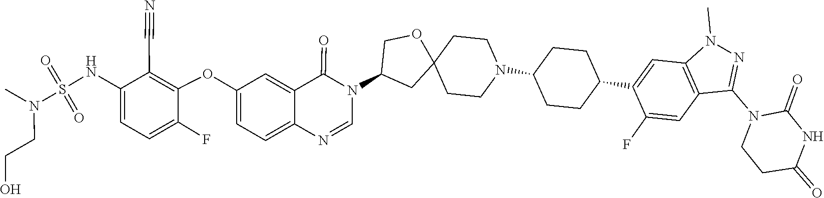 [SMILES: CN(CCO)S(=O)(=O)Nc1ccc(F)c(Oc2ccc3ncn([C@H]4COC5(CCN([C@H]6CC[C@@H](c7cc8c(cc7F)c(N7CCC(=O)NC7=O)nn8C)CC6)CC5)C4)c(=O)c3c2)c1C#N]

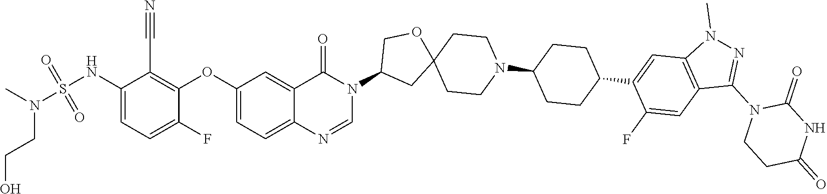 [SMILES: CN(CCO)S(=O)(=O)Nc1ccc(F)c(Oc2ccc3ncn([C@H]4COC5(CCN([C@H]6CC[C@H](c7cc8c(cc7F)c(N7CCC(=O)NC7=O)nn8C)CC6)CC5)C4)c(=O)c3c2)c1C#N]

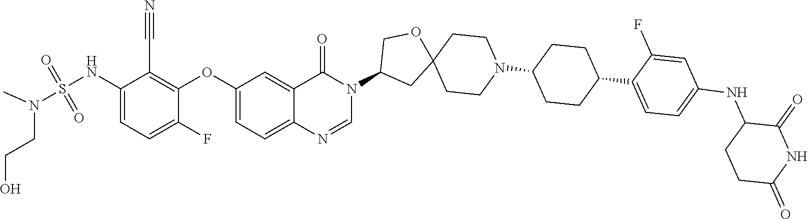 [SMILES: CN(CCO)S(=O)(=O)Nc1ccc(F)c(Oc2ccc3ncn([C@H]4COC5(CCN([C@H]6CC[C@@H](c7ccc(NC8CCC(=O)NC8=O)cc7F)CC6)CC5)C4)c(=O)c3c2)c1C#N]

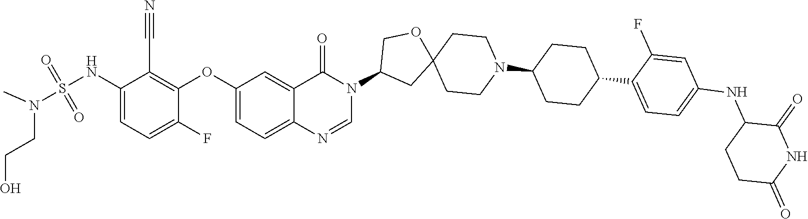 [SMILES: CN(CCO)S(=O)(=O)Nc1ccc(F)c(Oc2ccc3ncn([C@H]4COC5(CCN([C@H]6CC[C@H](c7ccc(NC8CCC(=O)NC8=O)cc7F)CC6)CC5)C4)c(=O)c3c2)c1C#N]

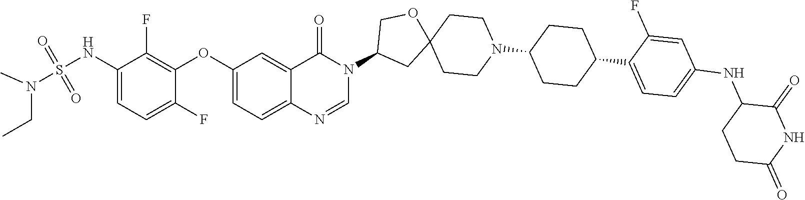 [SMILES: CCN(C)S(=O)(=O)Nc1ccc(F)c(Oc2ccc3ncn([C@H]4COC5(CCN([C@H]6CC[C@@H](c7ccc(NC8CCC(=O)NC8=O)cc7F)CC6)CC5)C4)c(=O)c3c2)c1F]

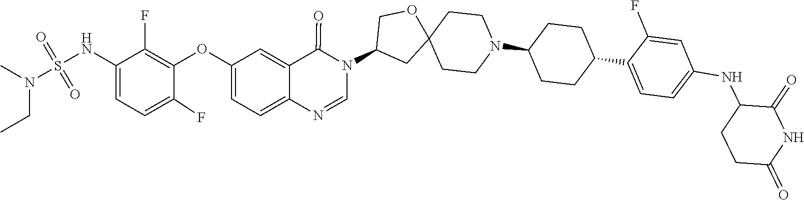 [SMILES: CCN(C)S(=O)(=O)Nc1ccc(F)c(Oc2ccc3ncn([C@H]4COC5(CCN([C@H]6CC[C@H](c7ccc(NC8CCC(=O)NC8=O)cc7F)CC6)CC5)C4)c(=O)c3c2)c1F]

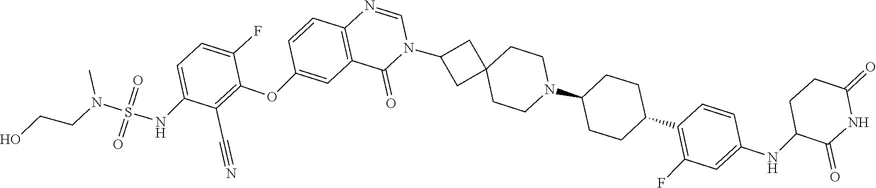 [SMILES: CN(CCO)S(=O)(=O)Nc1ccc(F)c(Oc2ccc3ncn(C4CC5(CCN([C@H]6CC[C@H](c7ccc(NC8CCC(=O)NC8=O)cc7F)CC6)CC5)C4)c(=O)c3c2)c1C#N]

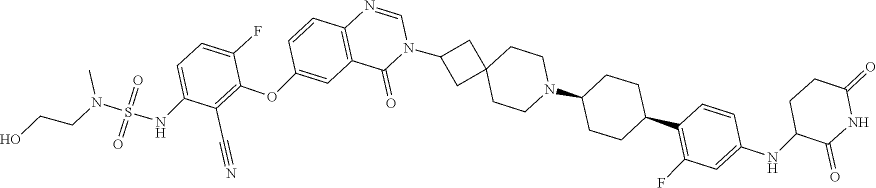 [SMILES: CN(CCO)S(=O)(=O)Nc1ccc(F)c(Oc2ccc3ncn(C4CC5(CCN([C@H]6CC[C@@H](c7ccc(NC8CCC(=O)NC8=O)cc7F)CC6)CC5)C4)c(=O)c3c2)c1C#N]

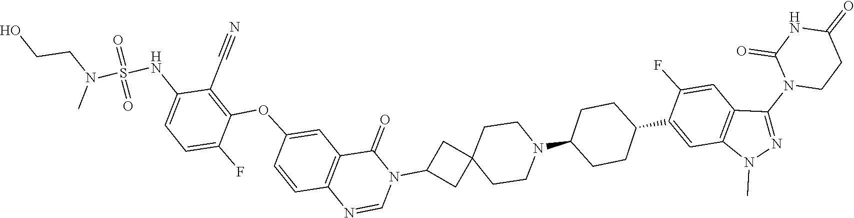 [SMILES: CN(CCO)S(=O)(=O)Nc1ccc(F)c(Oc2ccc3ncn(C4CC5(CCN([C@H]6CC[C@H](c7cc8c(cc7F)c(N7CCC(=O)NC7=O)nn8C)CC6)CC5)C4)c(=O)c3c2)c1C#N]

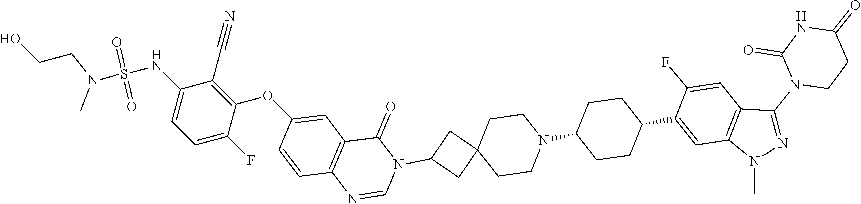 [SMILES: CN(CCO)S(=O)(=O)Nc1ccc(F)c(Oc2ccc3ncn(C4CC5(CCN([C@H]6CC[C@@H](c7cc8c(cc7F)c(N7CCC(=O)NC7=O)nn8C)CC6)CC5)C4)c(=O)c3c2)c1C#N]

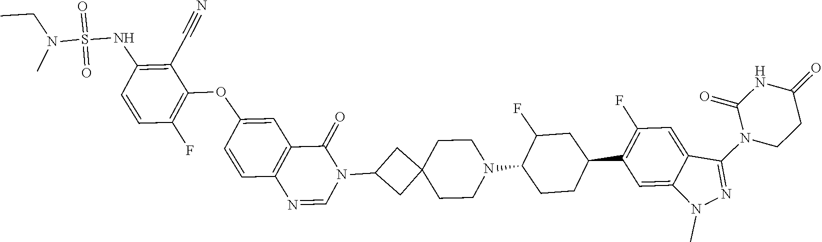 [SMILES: CCN(C)S(=O)(=O)Nc1ccc(F)c(Oc2ccc3ncn(C4CC5(CCN([C@H]6CC[C@H](c7cc8c(cc7F)c(N7CCC(=O)NC7=O)nn8C)CC6F)CC5)C4)c(=O)c3c2)c1C#N]

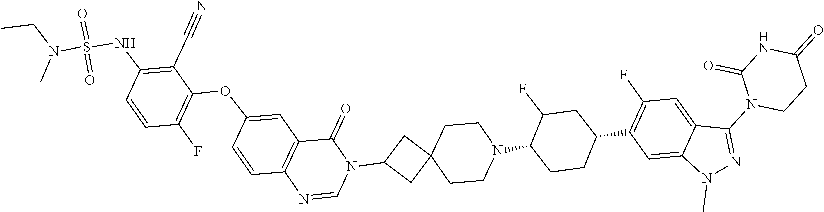 [SMILES: CCN(C)S(=O)(=O)Nc1ccc(F)c(Oc2ccc3ncn(C4CC5(CCN([C@H]6CC[C@@H](c7cc8c(cc7F)c(N7CCC(=O)NC7=O)nn8C)CC6F)CC5)C4)c(=O)c3c2)c1C#N]

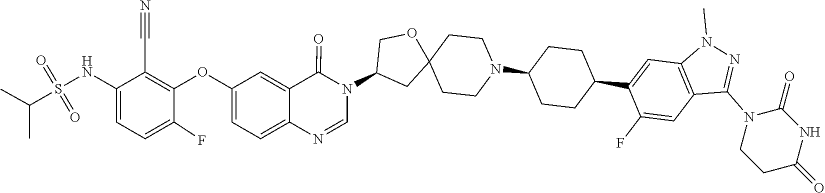 [SMILES: CC(C)S(=O)(=O)Nc1ccc(F)c(Oc2ccc3ncn([C@H]4COC5(CCN([C@H]6CC[C@@H](c7cc8c(cc7F)c(N7CCC(=O)NC7=O)nn8C)CC6)CC5)C4)c(=O)c3c2)c1C#N]

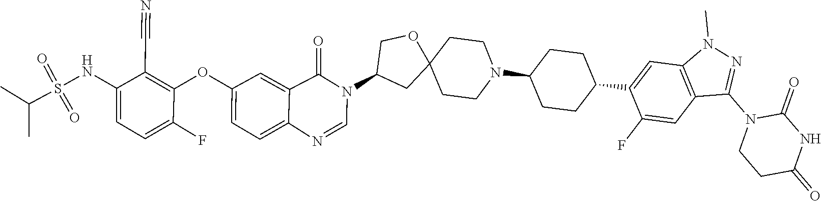 [SMILES: CC(C)S(=O)(=O)Nc1ccc(F)c(Oc2ccc3ncn([C@H]4COC5(CCN([C@H]6CC[C@H](c7cc8c(cc7F)c(N7CCC(=O)NC7=O)nn8C)CC6)CC5)C4)c(=O)c3c2)c1C#N]

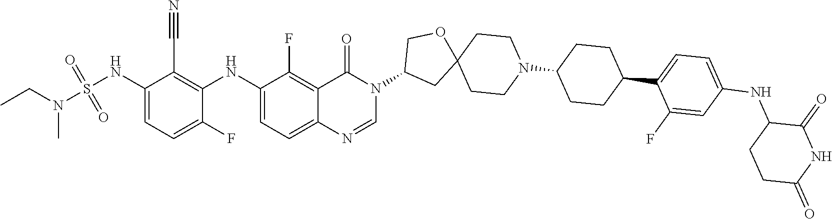 [SMILES: CCN(C)S(=O)(=O)Nc1ccc(F)c(Nc2ccc3ncn([C@@H]4COC5(CCN([C@H]6CC[C@H](c7ccc(NC8CCC(=O)NC8=O)cc7F)CC6)CC5)C4)c(=O)c3c2F)c1C#N]

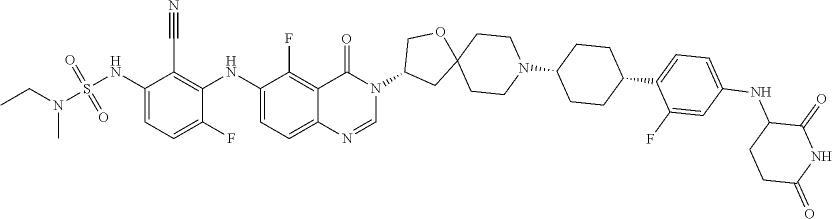 [SMILES: CCN(C)S(=O)(=O)Nc1ccc(F)c(Nc2ccc3ncn([C@@H]4COC5(CCN([C@H]6CC[C@@H](c7ccc(NC8CCC(=O)NC8=O)cc7F)CC6)CC5)C4)c(=O)c3c2F)c1C#N]

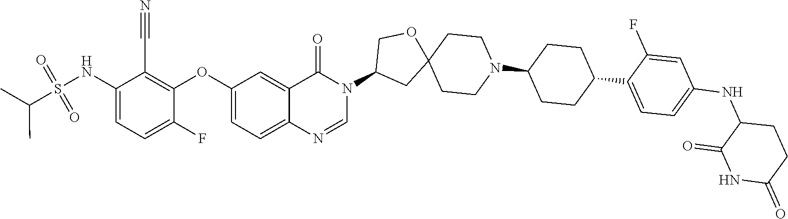 [SMILES: CC(C)S(=O)(=O)Nc1ccc(F)c(Oc2ccc3ncn([C@H]4COC5(CCN([C@H]6CC[C@H](c7ccc(NC8CCC(=O)NC8=O)cc7F)CC6)CC5)C4)c(=O)c3c2)c1C#N]

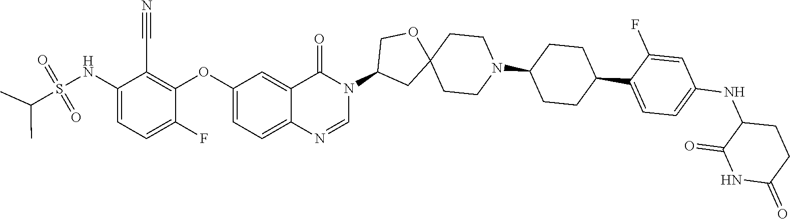 [SMILES: CC(C)S(=O)(=O)Nc1ccc(F)c(Oc2ccc3ncn([C@H]4COC5(CCN([C@H]6CC[C@@H](c7ccc(NC8CCC(=O)NC8=O)cc7F)CC6)CC5)C4)c(=O)c3c2)c1C#N]

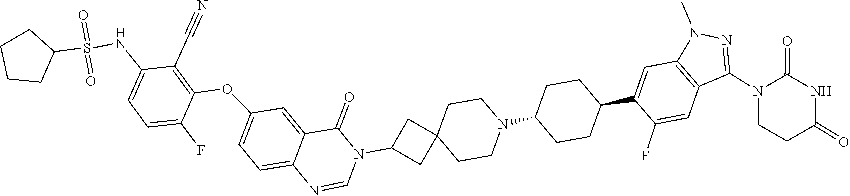 [SMILES: Cn1nc(N2CCC(=O)NC2=O)c2cc(F)c([C@H]3CC[C@H](N4CCC5(CC4)CC(n4cnc6ccc(Oc7c(F)ccc(NS(=O)(=O)C8CCCC8)c7C#N)cc6c4=O)C5)CC3)cc21]

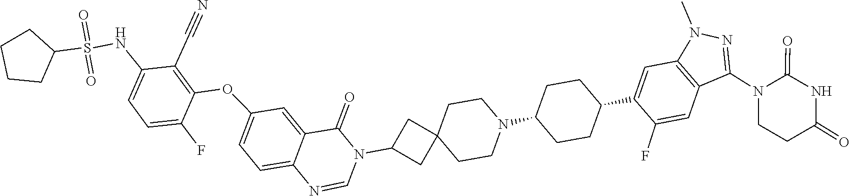 [SMILES: Cn1nc(N2CCC(=O)NC2=O)c2cc(F)c([C@H]3CC[C@@H](N4CCC5(CC4)CC(n4cnc6ccc(Oc7c(F)ccc(NS(=O)(=O)C8CCCC8)c7C#N)cc6c4=O)C5)CC3)cc21]

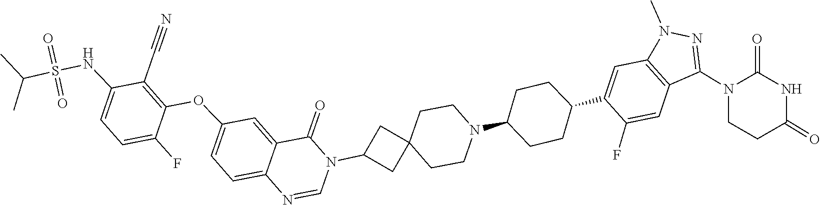 [SMILES: CC(C)S(=O)(=O)Nc1ccc(F)c(Oc2ccc3ncn(C4CC5(CCN([C@H]6CC[C@H](c7cc8c(cc7F)c(N7CCC(=O)NC7=O)nn8C)CC6)CC5)C4)c(=O)c3c2)c1C#N]

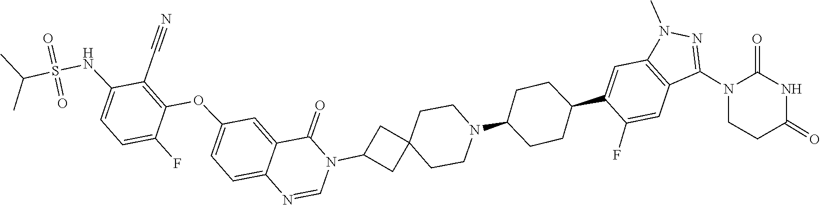 [SMILES: CC(C)S(=O)(=O)Nc1ccc(F)c(Oc2ccc3ncn(C4CC5(CCN([C@H]6CC[C@@H](c7cc8c(cc7F)c(N7CCC(=O)NC7=O)nn8C)CC6)CC5)C4)c(=O)c3c2)c1C#N]

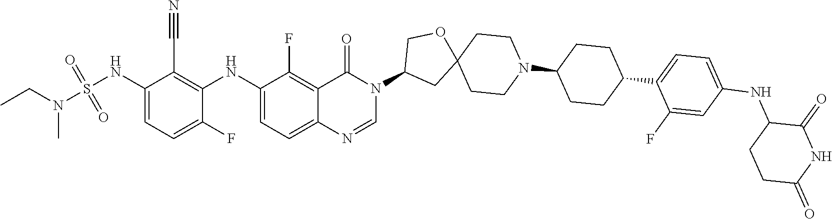 [SMILES: CCN(C)S(=O)(=O)Nc1ccc(F)c(Nc2ccc3ncn([C@H]4COC5(CCN([C@H]6CC[C@H](c7ccc(NC8CCC(=O)NC8=O)cc7F)CC6)CC5)C4)c(=O)c3c2F)c1C#N]

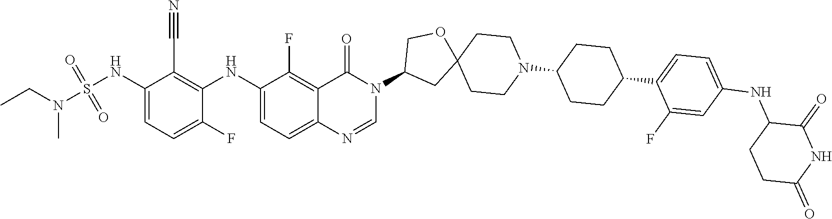 [SMILES: CCN(C)S(=O)(=O)Nc1ccc(F)c(Nc2ccc3ncn([C@H]4COC5(CCN([C@H]6CC[C@@H](c7ccc(NC8CCC(=O)NC8=O)cc7F)CC6)CC5)C4)c(=O)c3c2F)c1C#N]

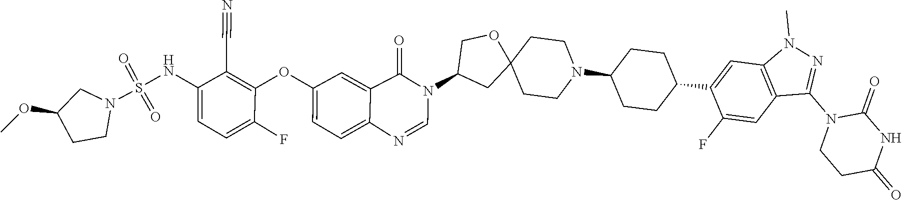 [SMILES: CO[C@@H]1CCN(S(=O)(=O)Nc2ccc(F)c(Oc3ccc4ncn([C@H]5COC6(CCN([C@H]7CC[C@H](c8cc9c(cc8F)c(N8CCC(=O)NC8=O)nn9C)CC7)CC6)C5)c(=O)c4c3)c2C#N)C1]

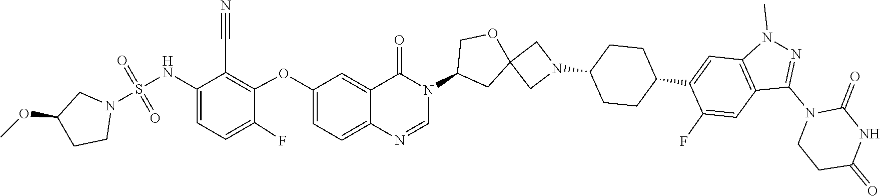 [SMILES: CO[C@@H]1CCN(S(=O)(=O)Nc2ccc(F)c(Oc3ccc4ncn([C@H]5COC6(C5)CN([C@H]5CC[C@@H](c7cc8c(cc7F)c(N7CCC(=O)NC7=O)nn8C)CC5)C6)c(=O)c4c3)c2C#N)C1]

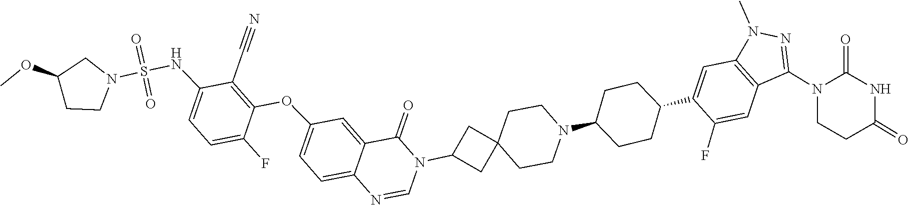 [SMILES: CO[C@@H]1CCN(S(=O)(=O)Nc2ccc(F)c(Oc3ccc4ncn(C5CC6(CCN([C@H]7CC[C@H](c8cc9c(cc8F)c(N8CCC(=O)NC8=O)nn9C)CC7)CC6)C5)c(=O)c4c3)c2C#N)C1]

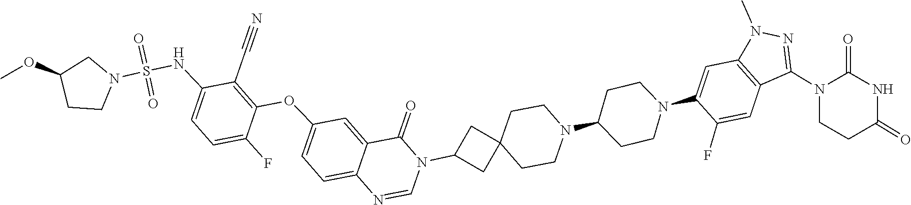

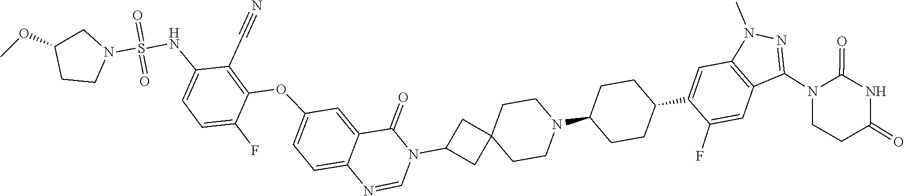 [SMILES: CO[C@H]1CCN(S(=O)(=O)Nc2ccc(F)c(Oc3ccc4ncn(C5CC6(CCN([C@H]7CC[C@H](c8cc9c(cc8F)c(N8CCC(=O)NC8=O)nn9C)CC7)CC6)C5)c(=O)c4c3)c2C#N)C1]

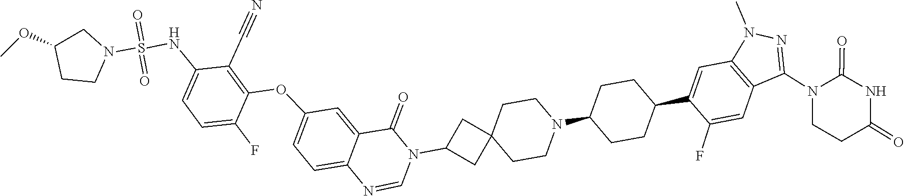 [SMILES: CO[C@H]1CCN(S(=O)(=O)Nc2ccc(F)c(Oc3ccc4ncn(C5CC6(CCN([C@H]7CC[C@@H](c8cc9c(cc8F)c(N8CCC(=O)NC8=O)nn9C)CC7)CC6)C5)c(=O)c4c3)c2C#N)C1]

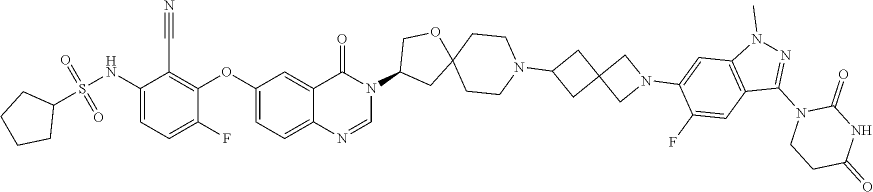 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CCCC9)c8C#N)cc7c5=O)CO6)C4)C3)cc21]

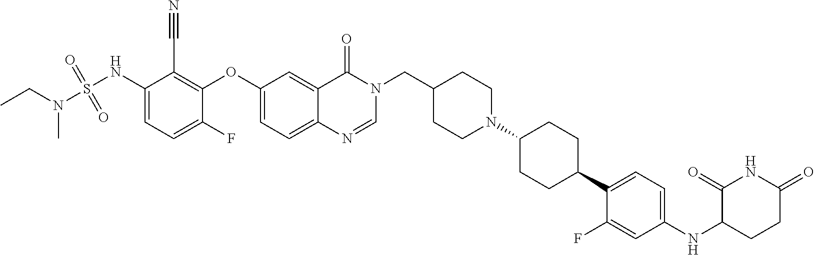 [SMILES: CCN(C)S(=O)(=O)Nc1ccc(F)c(Oc2ccc3ncn(CC4CCN([C@H]5CC[C@H](c6ccc(NC7CCC(=O)NC7=O)cc6F)CC5)CC4)c(=O)c3c2)c1C#N]

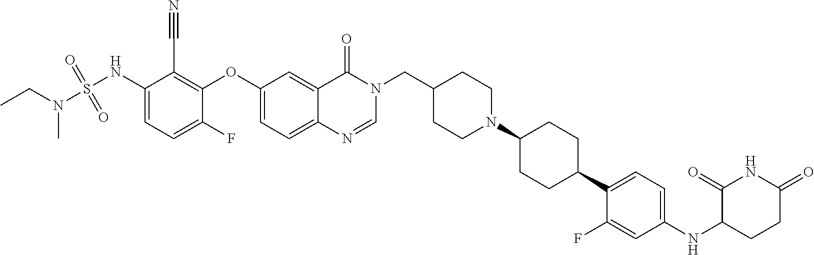 [SMILES: CCN(C)S(=O)(=O)Nc1ccc(F)c(Oc2ccc3ncn(CC4CCN([C@H]5CC[C@@H](c6ccc(NC7CCC(=O)NC7=O)cc6F)CC5)CC4)c(=O)c3c2)c1C#N]

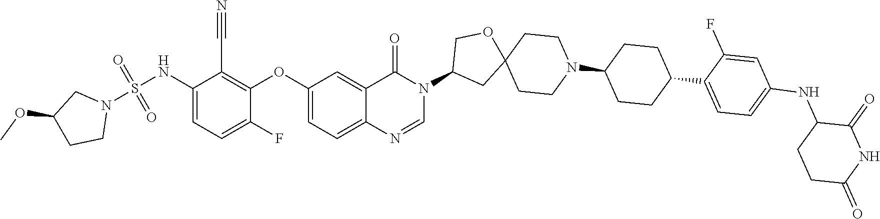 [SMILES: CO[C@@H]1CCN(S(=O)(=O)Nc2ccc(F)c(Oc3ccc4ncn([C@H]5COC6(CCN([C@H]7CC[C@H](c8ccc(NC9CCC(=O)NC9=O)cc8F)CC7)CC6)C5)c(=O)c4c3)c2C#N)C1]

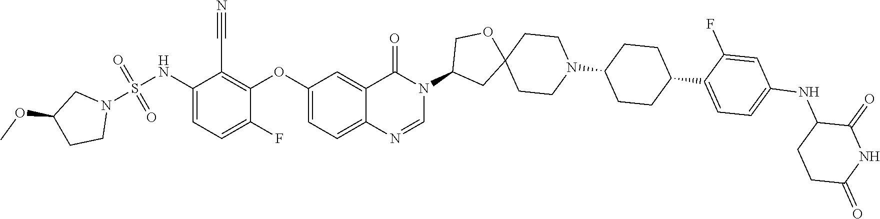 [SMILES: CO[C@@H]1CCN(S(=O)(=O)Nc2ccc(F)c(Oc3ccc4ncn([C@H]5COC6(CCN([C@H]7CC[C@@H](c8ccc(NC9CCC(=O)NC9=O)cc8F)CC7)CC6)C5)c(=O)c4c3)c2C#N)C1]

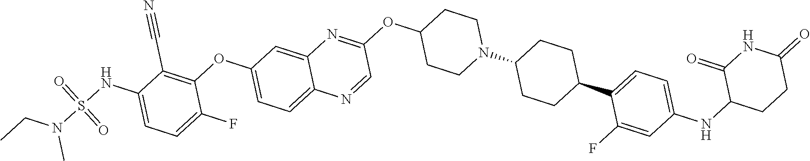 [SMILES: CCN(C)S(=O)(=O)Nc1ccc(F)c(Oc2ccc3ncc(OC4CCN([C@H]5CC[C@H](c6ccc(NC7CCC(=O)NC7=O)cc6F)CC5)CC4)nc3c2)c1C#N]

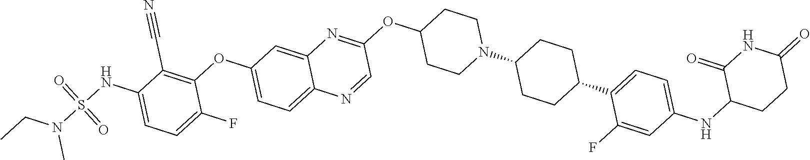 [SMILES: CCN(C)S(=O)(=O)Nc1ccc(F)c(Oc2ccc3ncc(OC4CCN([C@H]5CC[C@@H](c6ccc(NC7CCC(=O)NC7=O)cc6F)CC5)CC4)nc3c2)c1C#N]

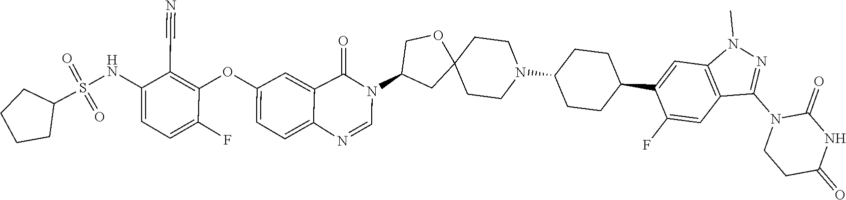 [SMILES: Cn1nc(N2CCC(=O)NC2=O)c2cc(F)c([C@H]3CC[C@H](N4CCC5(CC4)C[C@@H](n4cnc6ccc(Oc7c(F)ccc(NS(=O)(=O)C8CCCC8)c7C#N)cc6c4=O)CO5)CC3)cc21]

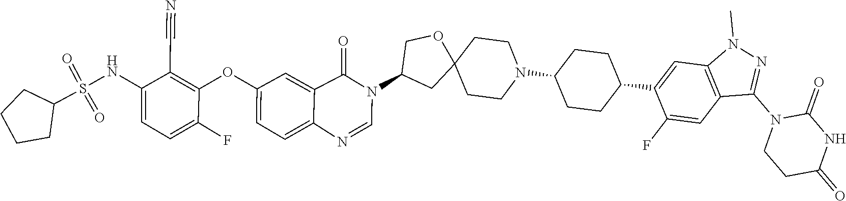 [SMILES: Cn1nc(N2CCC(=O)NC2=O)c2cc(F)c([C@H]3CC[C@@H](N4CCC5(CC4)C[C@@H](n4cnc6ccc(Oc7c(F)ccc(NS(=O)(=O)C8CCCC8)c7C#N)cc6c4=O)CO5)CC3)cc21]

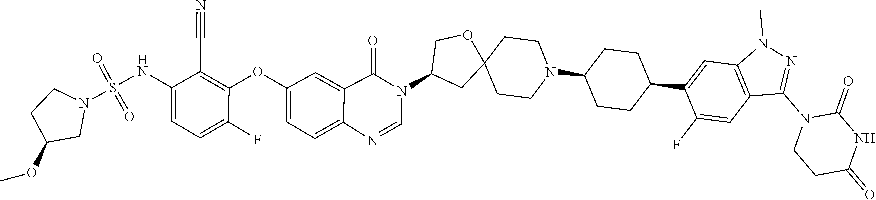 [SMILES: CO[C@H]1CCN(S(=O)(=O)Nc2ccc(F)c(Oc3ccc4ncn([C@H]5COC6(CCN([C@H]7CC[C@@H](c8cc9c(cc8F)c(N8CCC(=O)NC8=O)nn9C)CC7)CC6)C5)c(=O)c4c3)c2C#N)C1]

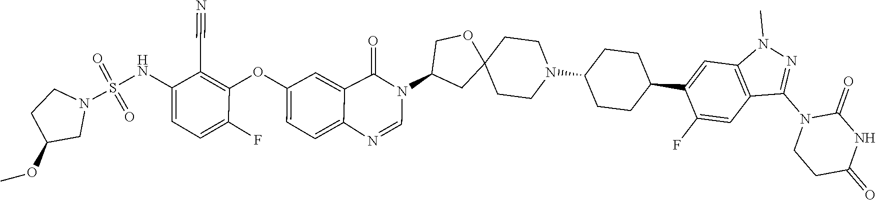 [SMILES: CO[C@H]1CCN(S(=O)(=O)Nc2ccc(F)c(Oc3ccc4ncn([C@H]5COC6(CCN([C@H]7CC[C@H](c8cc9c(cc8F)c(N8CCC(=O)NC8=O)nn9C)CC7)CC6)C5)c(=O)c4c3)c2C#N)C1]

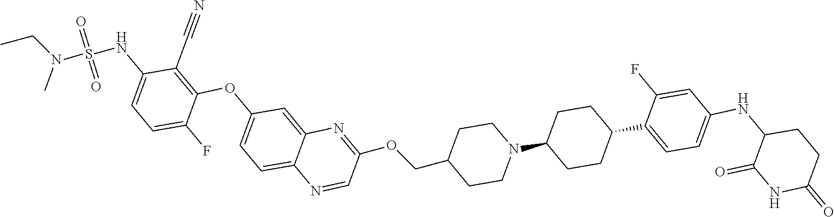 [SMILES: CCN(C)S(=O)(=O)Nc1ccc(F)c(Oc2ccc3ncc(OCC4CCN([C@H]5CC[C@H](c6ccc(NC7CCC(=O)NC7=O)cc6F)CC5)CC4)nc3c2)c1C#N]

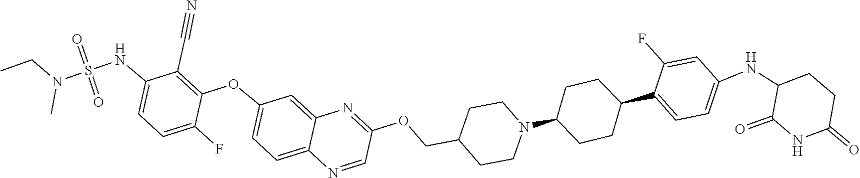 [SMILES: CCN(C)S(=O)(=O)Nc1ccc(F)c(Oc2ccc3ncc(OCC4CCN([C@H]5CC[C@@H](c6ccc(NC7CCC(=O)NC7=O)cc6F)CC5)CC4)nc3c2)c1C#N]

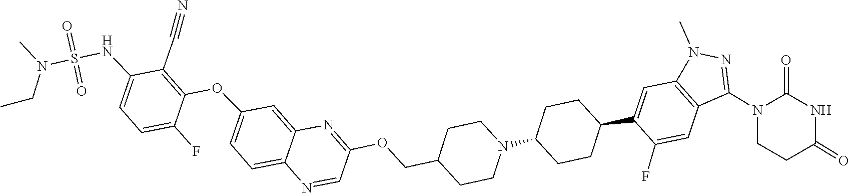 [SMILES: CCN(C)S(=O)(=O)Nc1ccc(F)c(Oc2ccc3ncc(OCC4CCN([C@H]5CC[C@H](c6cc7c(cc6F)c(N6CCC(=O)NC6=O)nn7C)CC5)CC4)nc3c2)c1C#N]

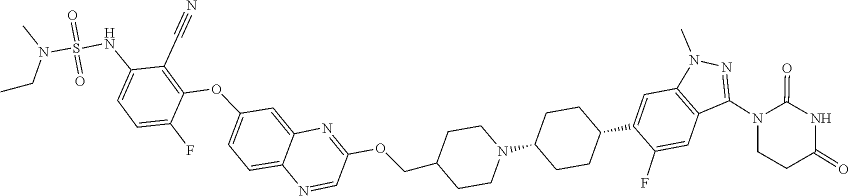 [SMILES: CCN(C)S(=O)(=O)Nc1ccc(F)c(Oc2ccc3ncc(OCC4CCN([C@H]5CC[C@@H](c6cc7c(cc6F)c(N6CCC(=O)NC6=O)nn7C)CC5)CC4)nc3c2)c1C#N]

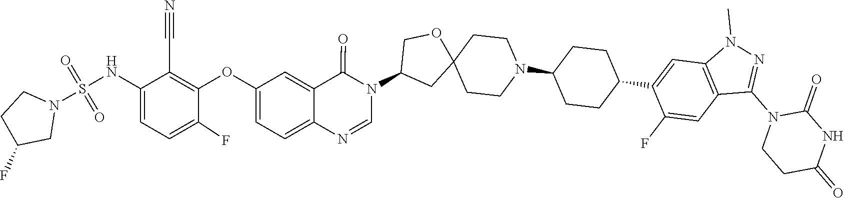 [SMILES: Cn1nc(N2CCC(=O)NC2=O)c2cc(F)c([C@H]3CC[C@H](N4CCC5(CC4)C[C@@H](n4cnc6ccc(Oc7c(F)ccc(NS(=O)(=O)N8CC[C@@H](F)C8)c7C#N)cc6c4=O)CO5)CC3)cc21]

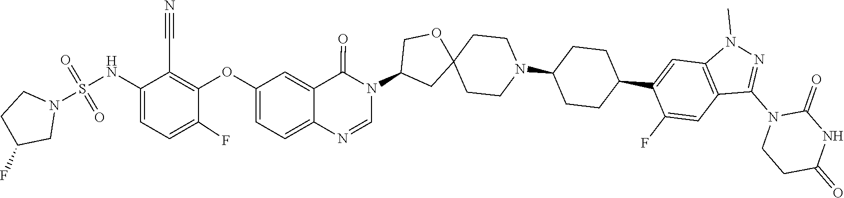 [SMILES: Cn1nc(N2CCC(=O)NC2=O)c2cc(F)c([C@H]3CC[C@@H](N4CCC5(CC4)C[C@@H](n4cnc6ccc(Oc7c(F)ccc(NS(=O)(=O)N8CC[C@@H](F)C8)c7C#N)cc6c4=O)CO5)CC3)cc21]

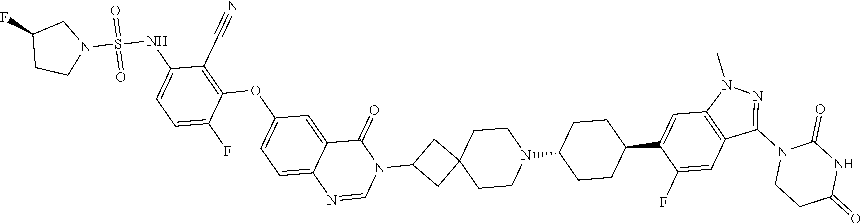 [SMILES: Cn1nc(N2CCC(=O)NC2=O)c2cc(F)c([C@H]3CC[C@H](N4CCC5(CC4)CC(n4cnc6ccc(Oc7c(F)ccc(NS(=O)(=O)N8CC[C@@H](F)C8)c7C#N)cc6c4=O)C5)CC3)cc21]

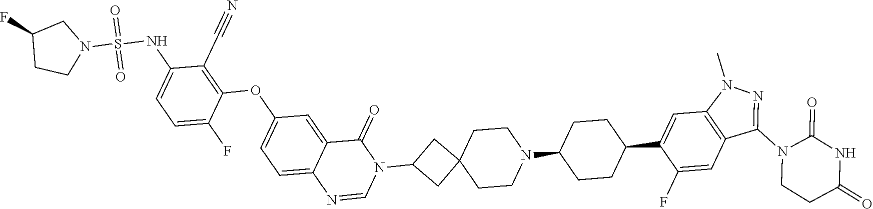 [SMILES: Cn1nc(N2CCC(=O)NC2=O)c2cc(F)c([C@H]3CC[C@@H](N4CCC5(CC4)CC(n4cnc6ccc(Oc7c(F)ccc(NS(=O)(=O)N8CC[C@@H](F)C8)c7C#N)cc6c4=O)C5)CC3)cc21]

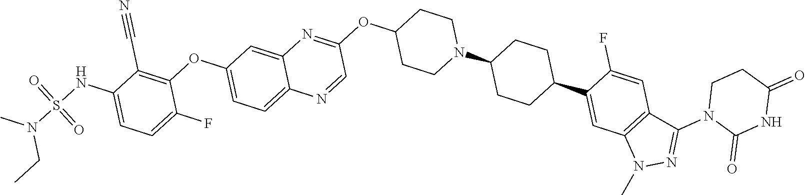 [SMILES: CCN(C)S(=O)(=O)Nc1ccc(F)c(Oc2ccc3ncc(OC4CCN([C@H]5CC[C@@H](c6cc7c(cc6F)c(N6CCC(=O)NC6=O)nn7C)CC5)CC4)nc3c2)c1C#N]

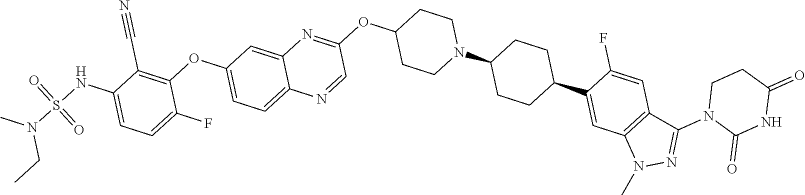 [SMILES: CCN(C)S(=O)(=O)Nc1ccc(F)c(Oc2ccc3ncc(OC4CCN([C@H]5CC[C@@H](c6cc7c(cc6F)c(N6CCC(=O)NC6=O)nn7C)CC5)CC4)nc3c2)c1C#N]

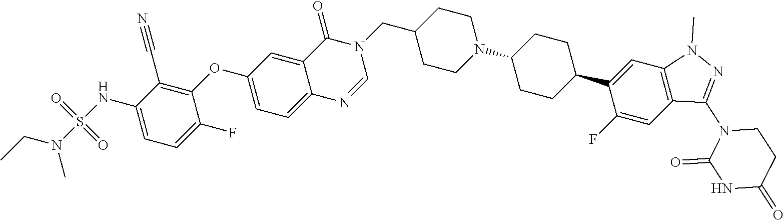 [SMILES: CCN(C)S(=O)(=O)Nc1ccc(F)c(Oc2ccc3ncn(CC4CCN([C@H]5CC[C@H](c6cc7c(cc6F)c(N6CCC(=O)NC6=O)nn7C)CC5)CC4)c(=O)c3c2)c1C#N]

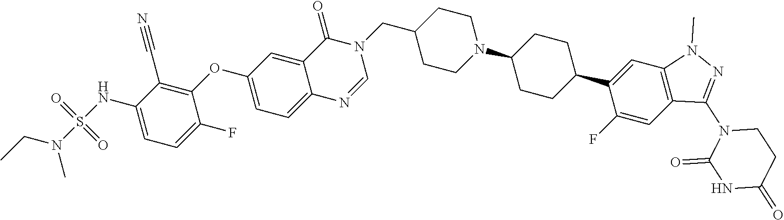 [SMILES: CCN(C)S(=O)(=O)Nc1ccc(F)c(Oc2ccc3ncn(CC4CCN([C@H]5CC[C@@H](c6cc7c(cc6F)c(N6CCC(=O)NC6=O)nn7C)CC5)CC4)c(=O)c3c2)c1C#N]

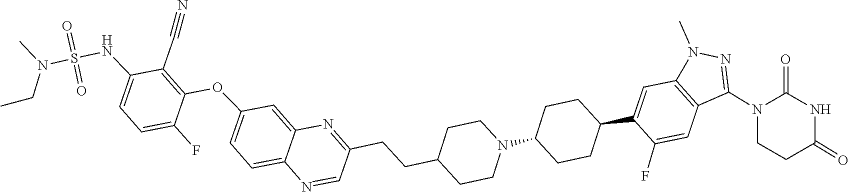 [SMILES: CCN(C)S(=O)(=O)Nc1ccc(F)c(Oc2ccc3ncc(CCC4CCN([C@H]5CC[C@H](c6cc7c(cc6F)c(N6CCC(=O)NC6=O)nn7C)CC5)CC4)nc3c2)c1C#N]

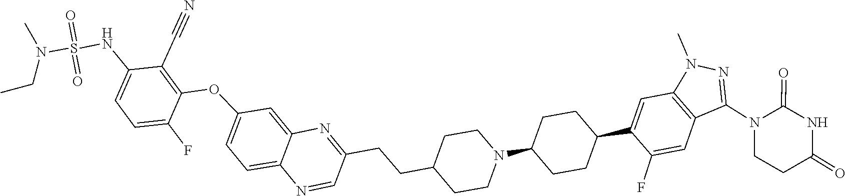 [SMILES: CCN(C)S(=O)(=O)Nc1ccc(F)c(Oc2ccc3ncc(CCC4CCN([C@H]5CC[C@@H](c6cc7c(cc6F)c(N6CCC(=O)NC6=O)nn7C)CC5)CC4)nc3c2)c1C#N]

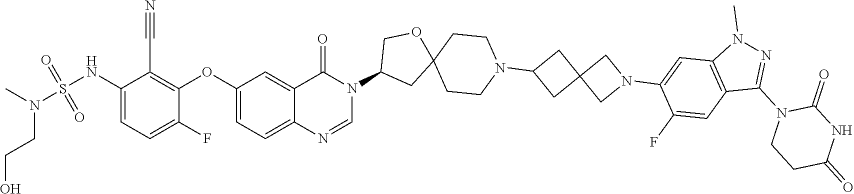 [SMILES: CN(CCO)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N]

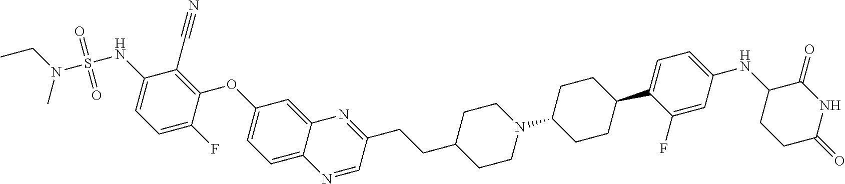 [SMILES: CCN(C)S(=O)(=O)Nc1ccc(F)c(Oc2ccc3ncc(CCC4CCN([C@H]5CC[C@H](c6ccc(NC7CCC(=O)NC7=O)cc6F)CC5)CC4)nc3c2)c1C#N]

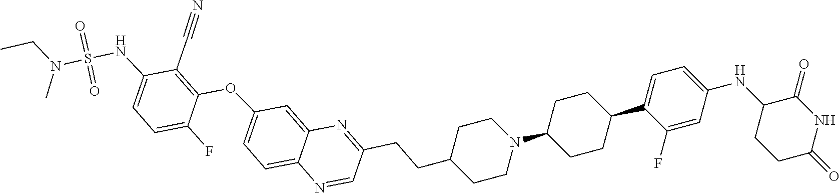 [SMILES: CCN(C)S(=O)(=O)Nc1ccc(F)c(Oc2ccc3ncc(CCC4CCN([C@H]5CC[C@@H](c6ccc(NC7CCC(=O)NC7=O)cc6F)CC5)CC4)nc3c2)c1C#N]

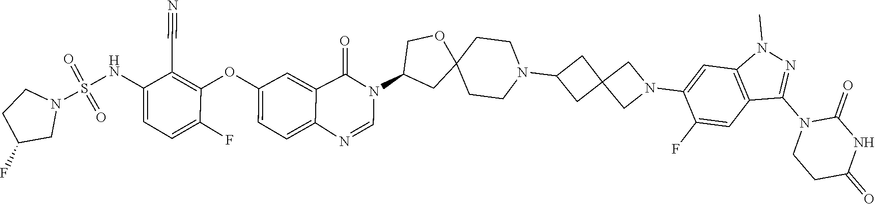 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC[C@@H](F)C9)c8C#N)cc7c5=O)CO6)C4)C3)cc21]

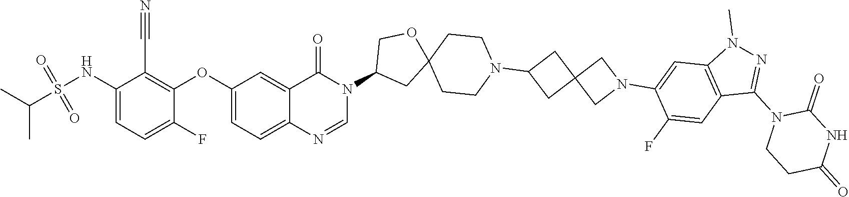 [SMILES: CC(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N]

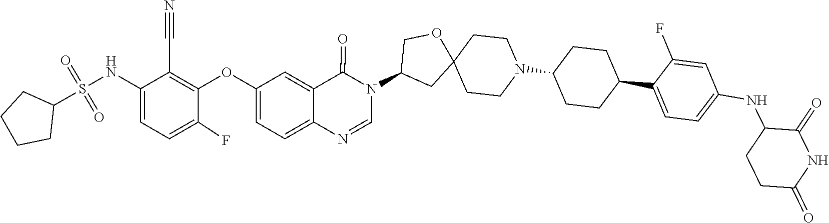 [SMILES: N#Cc1c(NS(=O)(=O)C2CCCC2)ccc(F)c1Oc1ccc2ncn([C@H]3COC4(CCN([C@H]5CC[C@H](c6ccc(NC7CCC(=O)NC7=O)cc6F)CC5)CC4)C3)c(=O)c2c1]

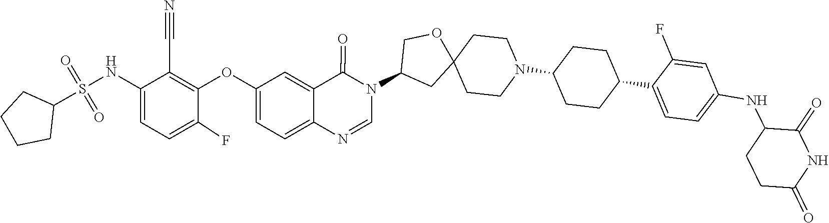 [SMILES: N#Cc1c(NS(=O)(=O)C2CCCC2)ccc(F)c1Oc1ccc2ncn([C@H]3COC4(CCN([C@H]5CC[C@@H](c6ccc(NC7CCC(=O)NC7=O)cc6F)CC5)CC4)C3)c(=O)c2c1]

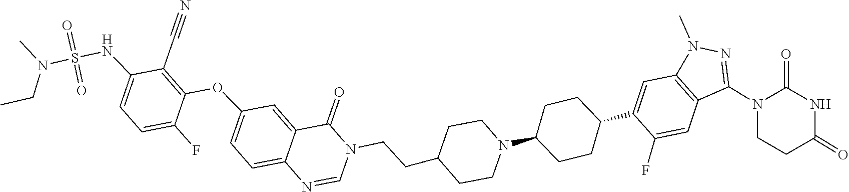 [SMILES: CCN(C)S(=O)(=O)Nc1ccc(F)c(Oc2ccc3ncn(CCC4CCN([C@H]5CC[C@H](c6cc7c(cc6F)c(N6CCC(=O)NC6=O)nn7C)CC5)CC4)c(=O)c3c2)c1C#N]

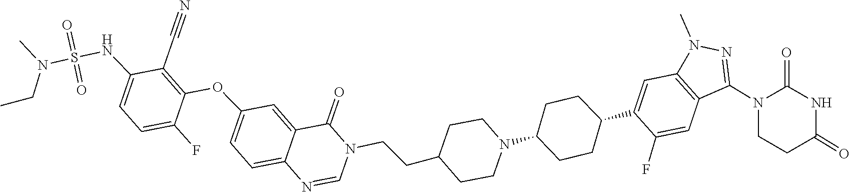 [SMILES: CCN(C)S(=O)(=O)Nc1ccc(F)c(Oc2ccc3ncn(CCC4CCN([C@H]5CC[C@@H](c6cc7c(cc6F)c(N6CCC(=O)NC6=O)nn7C)CC5)CC4)c(=O)c3c2)c1C#N]

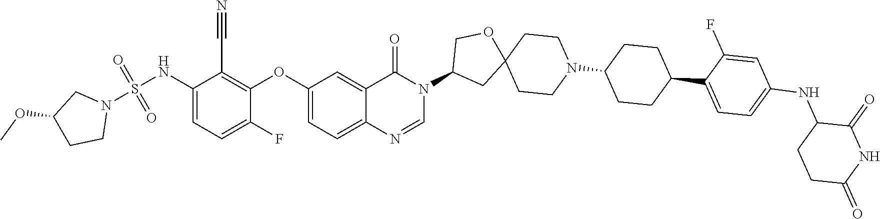 [SMILES: CO[C@H]1CCN(S(=O)(=O)Nc2ccc(F)c(Oc3ccc4ncn([C@H]5COC6(CCN([C@H]7CC[C@H](c8ccc(NC9CCC(=O)NC9=O)cc8F)CC7)CC6)C5)c(=O)c4c3)c2C#N)C1]

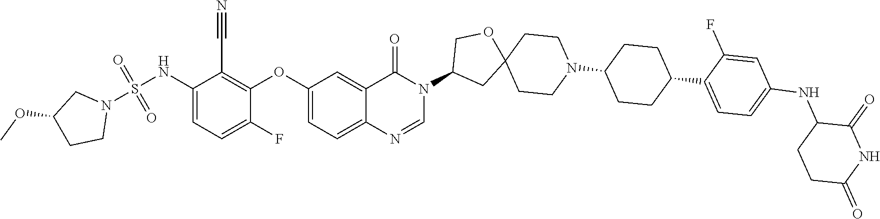 [SMILES: CO[C@H]1CCN(S(=O)(=O)Nc2ccc(F)c(Oc3ccc4ncn([C@H]5COC6(CCN([C@H]7CC[C@@H](c8ccc(NC9CCC(=O)NC9=O)cc8F)CC7)CC6)C5)c(=O)c4c3)c2C#N)C1]

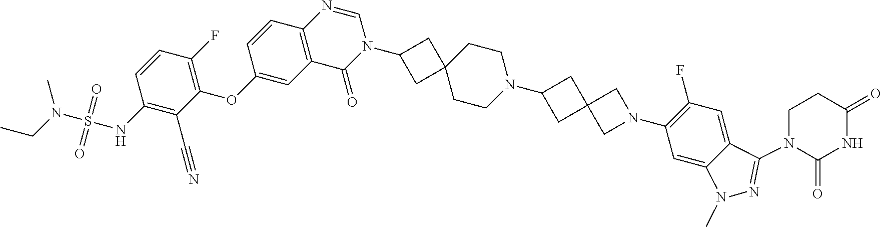 [SMILES: CCN(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1C#N]

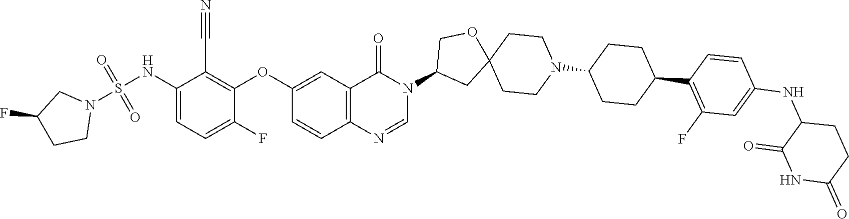 [SMILES: N#Cc1c(NS(=O)(=O)N2CC[C@@H](F)C2)ccc(F)c1Oc1ccc2ncn([C@H]3COC4(CCN([C@H]5CC[C@H](c6ccc(NC7CCC(=O)NC7=O)cc6F)CC5)CC4)C3)c(=O)c2c1]

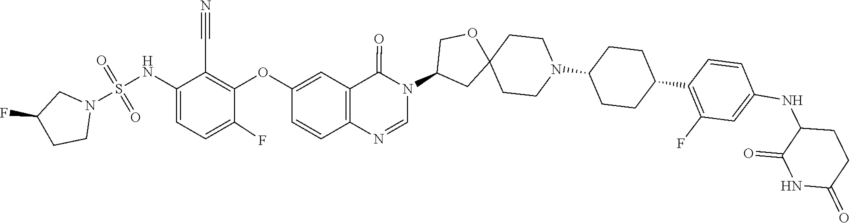 [SMILES: N#Cc1c(NS(=O)(=O)N2CC[C@@H](F)C2)ccc(F)c1Oc1ccc2ncn([C@H]3COC4(CCN([C@H]5CC[C@@H](c6ccc(NC7CCC(=O)NC7=O)cc6F)CC5)CC4)C3)c(=O)c2c1]

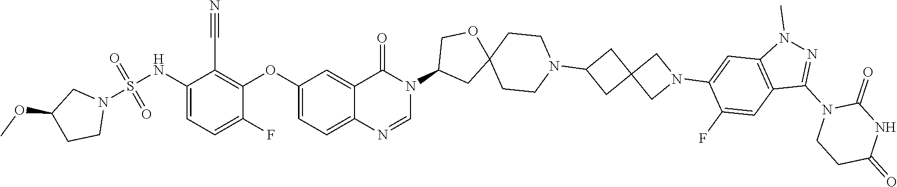 [SMILES: CO[C@@H]1CCN(S(=O)(=O)Nc2ccc(F)c(Oc3ccc4ncn([C@H]5COC6(CCN(C7CC8(C7)CN(c7cc9c(cc7F)c(N7CCC(=O)NC7=O)nn9C)C8)CC6)C5)c(=O)c4c3)c2C#N)C1]

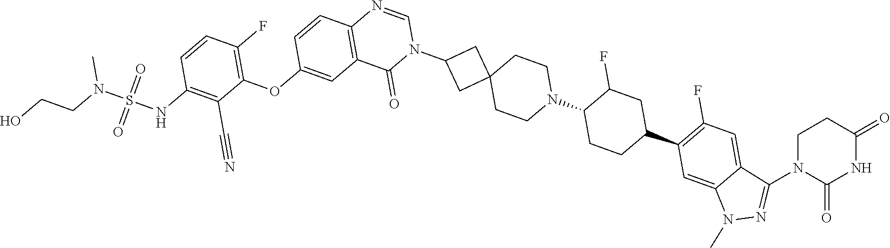

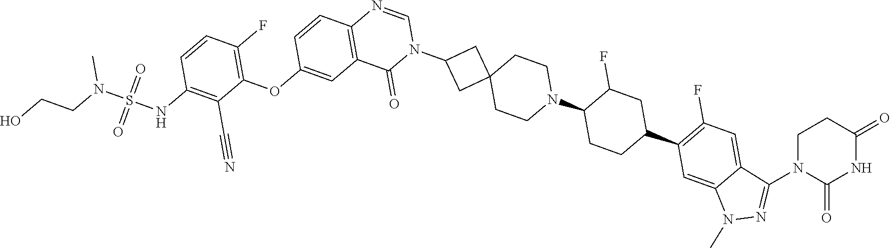

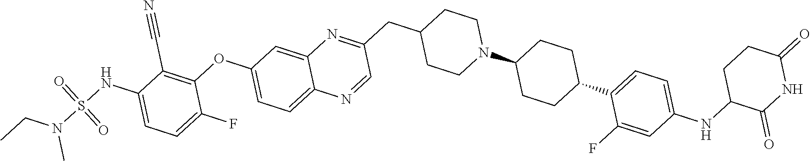 [SMILES: CCN(C)S(=O)(=O)Nc1ccc(F)c(Oc2ccc3ncc(CC4CCN([C@H]5CC[C@H](c6ccc(NC7CCC(=O)NC7=O)cc6F)CC5)CC4)nc3c2)c1C#N]

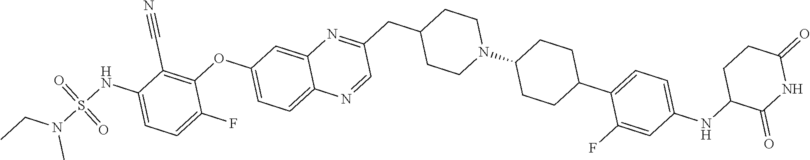

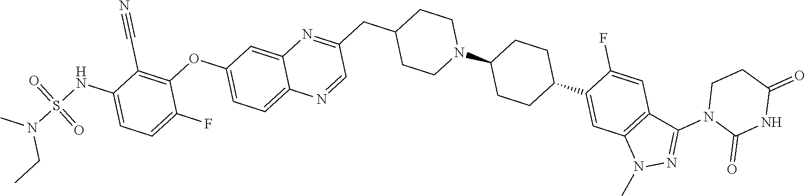 [SMILES: CCN(C)S(=O)(=O)Nc1ccc(F)c(Oc2ccc3ncc(CC4CCN([C@H]5CC[C@H](c6cc7c(cc6F)c(N6CCC(=O)NC6=O)nn7C)CC5)CC4)nc3c2)c1C#N]

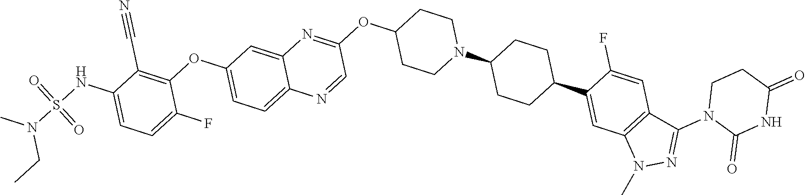 [SMILES: CCN(C)S(=O)(=O)Nc1ccc(F)c(Oc2ccc3ncc(OC4CCN([C@H]5CC[C@@H](c6cc7c(cc6F)c(N6CCC(=O)NC6=O)nn7C)CC5)CC4)nc3c2)c1C#N]

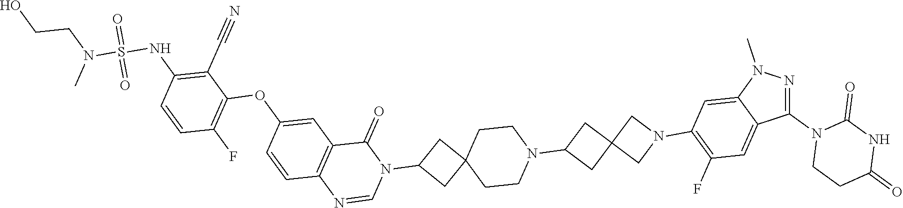 [SMILES: CN(CCO)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1C#N]

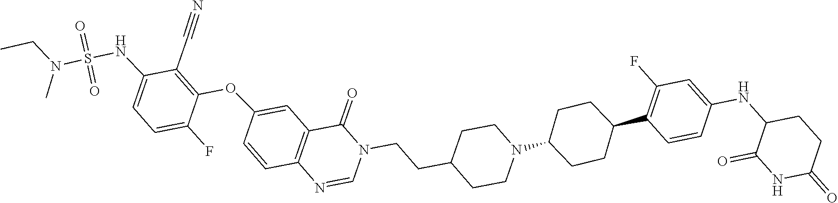 [SMILES: CCN(C)S(=O)(=O)Nc1ccc(F)c(Oc2ccc3ncn(CCC4CCN([C@H]5CC[C@H](c6ccc(NC7CCC(=O)NC7=O)cc6F)CC5)CC4)c(=O)c3c2)c1C#N]

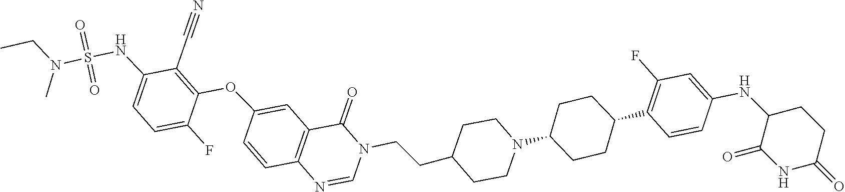 [SMILES: CCN(C)S(=O)(=O)Nc1ccc(F)c(Oc2ccc3ncn(CCC4CCN([C@H]5CC[C@@H](c6ccc(NC7CCC(=O)NC7=O)cc6F)CC5)CC4)c(=O)c3c2)c1C#N]

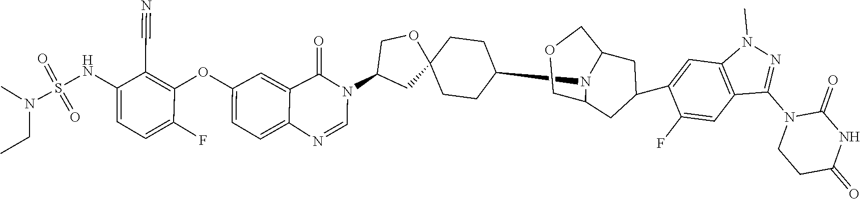 [SMILES: CCN(C)S(=O)(=O)Nc1ccc(F)c(Oc2ccc3ncn([C@H]4CO[C@]5(CC[C@@H](N6C7COCC6CC(c6cc8c(cc6F)c(N6CCC(=O)NC6=O)nn8C)C7)CC5)C4)c(=O)c3c2)c1C#N]

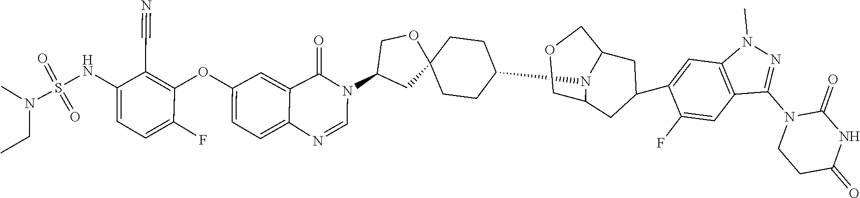 [SMILES: CCN(C)S(=O)(=O)Nc1ccc(F)c(Oc2ccc3ncn([C@H]4CO[C@]5(CC[C@H](N6C7COCC6CC(c6cc8c(cc6F)c(N6CCC(=O)NC6=O)nn8C)C7)CC5)C4)c(=O)c3c2)c1C#N]

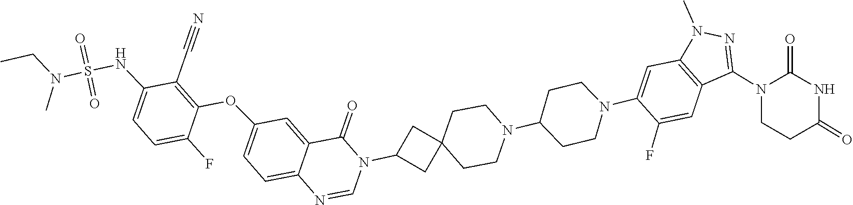 [SMILES: CCN(C)S(=O)(=O)Nc1ccc(F)c(Oc2ccc3ncn(C4CC5(CCN(C6CCN(c7cc8c(cc7F)c(N7CCC(=O)NC7=O)nn8C)CC6)CC5)C4)c(=O)c3c2)c1C#N]

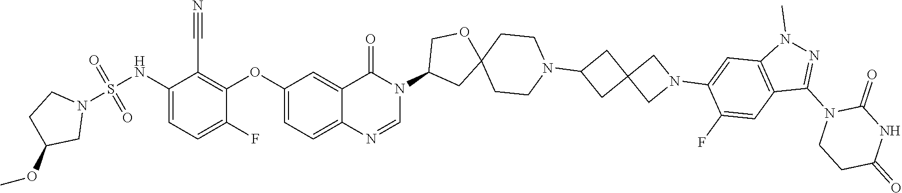 [SMILES: CO[C@H]1CCN(S(=O)(=O)Nc2ccc(F)c(Oc3ccc4ncn([C@H]5COC6(CCN(C7CC8(C7)CN(c7cc9c(cc7F)c(N7CCC(=O)NC7=O)nn9C)C8)CC6)C5)c(=O)c4c3)c2C#N)C1]

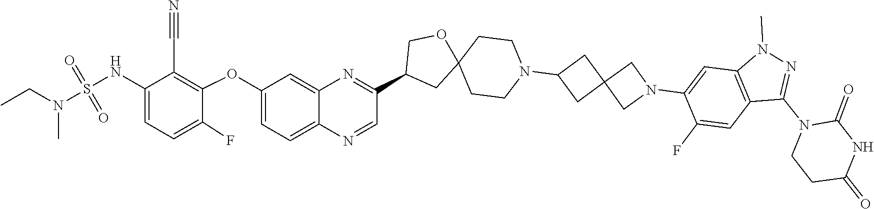 [SMILES: CCN(C)S(=O)(=O)Nc1ccc(F)c(Oc2ccc3ncc([C@H]4COC5(CCN(C6CC7(C6)CN(c6cc8c(cc6F)c(N6CCC(=O)NC6=O)nn8C)C7)CC5)C4)nc3c2)c1C#N]

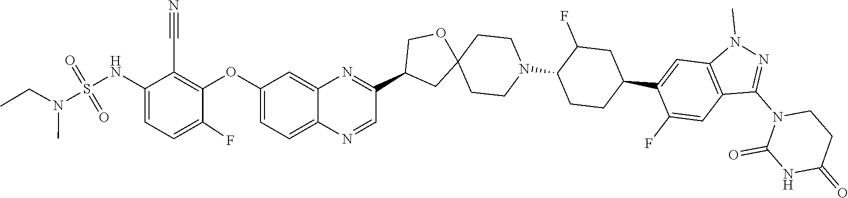 [SMILES: CCN(C)S(=O)(=O)Nc1ccc(F)c(Oc2ccc3ncc([C@H]4COC5(CCN([C@H]6CC[C@H](c7cc8c(cc7F)c(N7CCC(=O)NC7=O)nn8C)CC6F)CC5)C4)nc3c2)c1C#N]

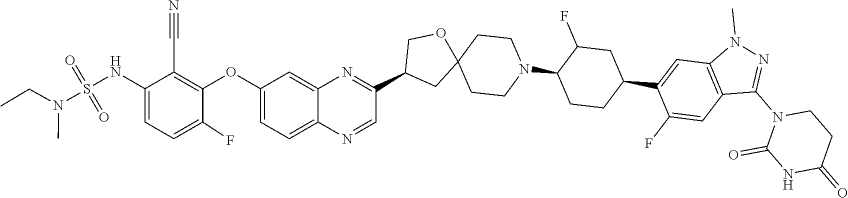 [SMILES: CCN(C)S(=O)(=O)Nc1ccc(F)c(Oc2ccc3ncc([C@H]4COC5(CCN([C@@H]6CC[C@H](c7cc8c(cc7F)c(N7CCC(=O)NC7=O)nn8C)CC6F)CC5)C4)nc3c2)c1C#N]

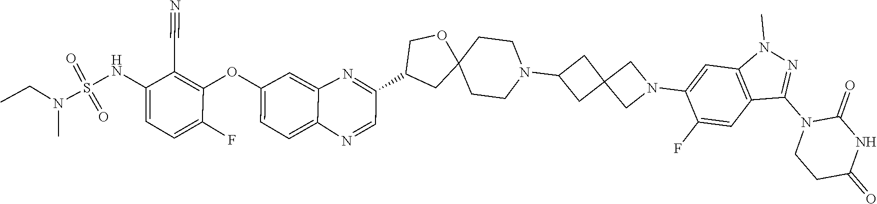

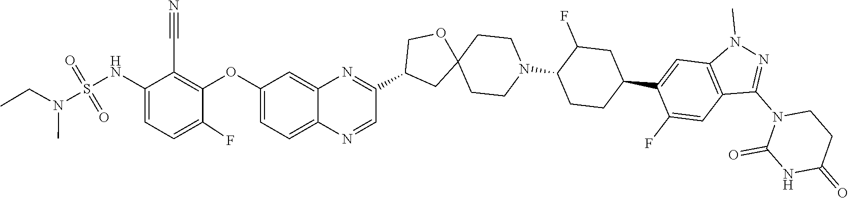 [SMILES: CCN(C)S(=O)(=O)Nc1ccc(F)c(Oc2ccc3ncc([C@@H]4COC5(CCN([C@H]6CC[C@H](c7cc8c(cc7F)c(N7CCC(=O)NC7=O)nn8C)CC6F)CC5)C4)nc3c2)c1C#N]

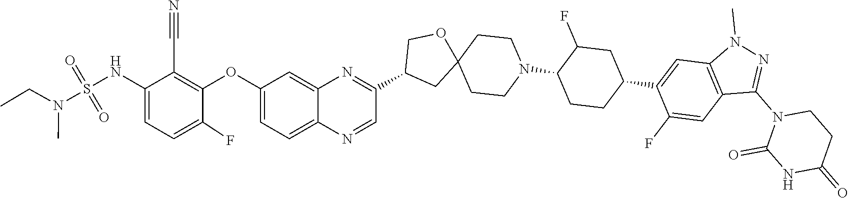 [SMILES: CCN(C)S(=O)(=O)Nc1ccc(F)c(Oc2ccc3ncc([C@@H]4COC5(CCN([C@H]6CC[C@@H](c7cc8c(cc7F)c(N7CCC(=O)NC7=O)nn8C)CC6F)CC5)C4)nc3c2)c1C#N]

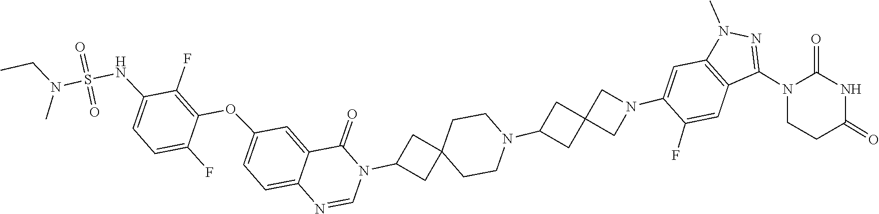 [SMILES: CCN(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1F]

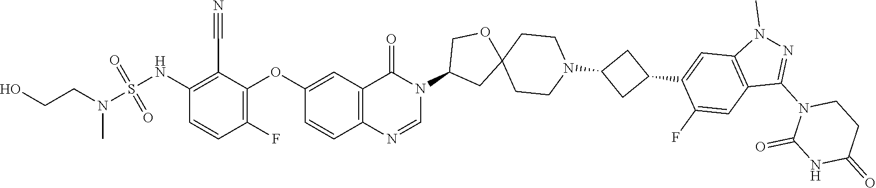 [SMILES: CN(CCO)S(=O)(=O)Nc1ccc(F)c(Oc2ccc3ncn([C@H]4COC5(CCN([C@H]6C[C@@H](c7cc8c(cc7F)c(N7CCC(=O)NC7=O)nn8C)C6)CC5)C4)c(=O)c3c2)c1C#N]

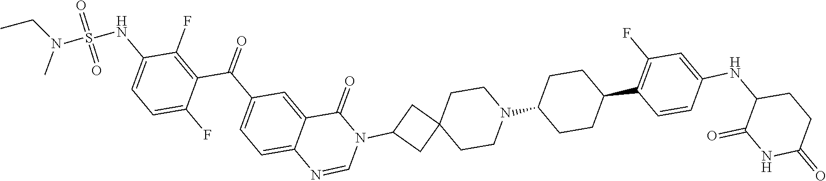 [SMILES: CCN(C)S(=O)(=O)Nc1ccc(F)c(C(=O)c2ccc3ncn(C4CC5(CCN([C@H]6CC[C@H](c7ccc(NC8CCC(=O)NC8=O)cc7F)CC6)CC5)C4)c(=O)c3c2)c1F]

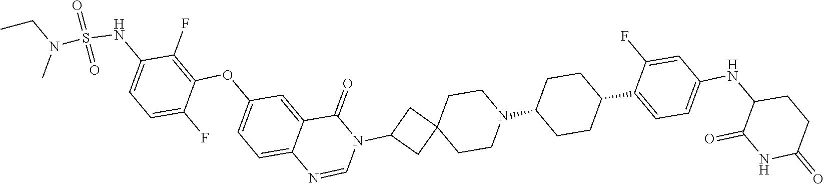 [SMILES: CCN(C)S(=O)(=O)Nc1ccc(F)c(Oc2ccc3ncn(C4CC5(CCN([C@H]6CC[C@@H](c7ccc(NC8CCC(=O)NC8=O)cc7F)CC6)CC5)C4)c(=O)c3c2)c1F]

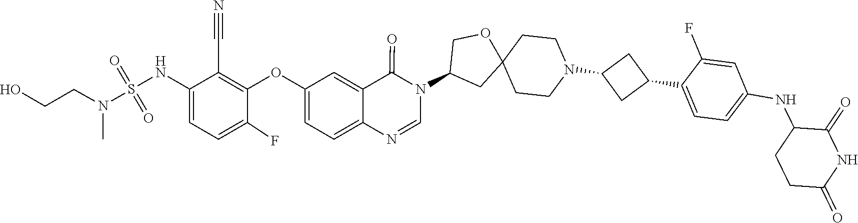 [SMILES: CN(CCO)S(=O)(=O)Nc1ccc(F)c(Oc2ccc3ncn([C@H]4COC5(CCN([C@H]6C[C@@H](c7ccc(NC8CCC(=O)NC8=O)cc7F)C6)CC5)C4)c(=O)c3c2)c1C#N]

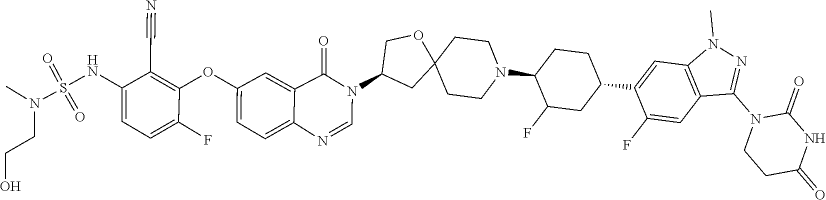 [SMILES: CN(CCO)S(=O)(=O)Nc1ccc(F)c(Oc2ccc3ncn([C@H]4COC5(CCN([C@H]6CC[C@H](c7cc8c(cc7F)c(N7CCC(=O)NC7=O)nn8C)CC6F)CC5)C4)c(=O)c3c2)c1C#N]

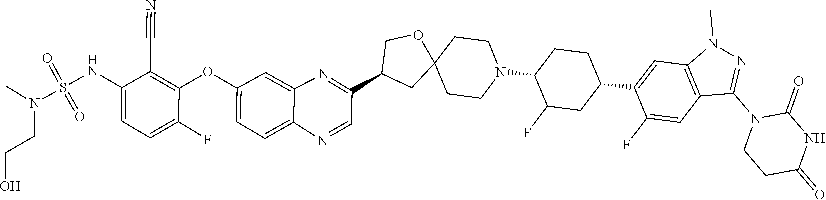 [SMILES: CN(CCO)S(=O)(=O)Nc1ccc(F)c(Oc2ccc3ncc([C@H]4COC5(CCN([C@@H]6CC[C@H](c7cc8c(cc7F)c(N7CCC(=O)NC7=O)nn8C)CC6F)CC5)C4)nc3c2)c1C#N]

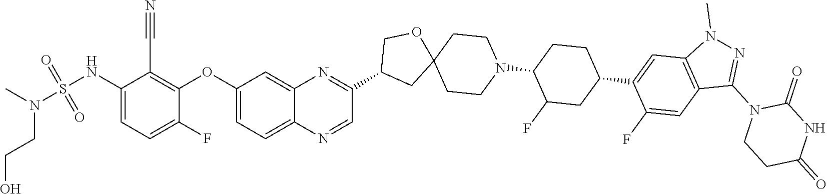 [SMILES: CN(CCO)S(=O)(=O)Nc1ccc(F)c(Oc2ccc3ncc([C@@H]4COC5(CCN([C@@H]6CC[C@H](c7cc8c(cc7F)c(N7CCC(=O)NC7=O)nn8C)CC6F)CC5)C4)nc3c2)c1C#N]

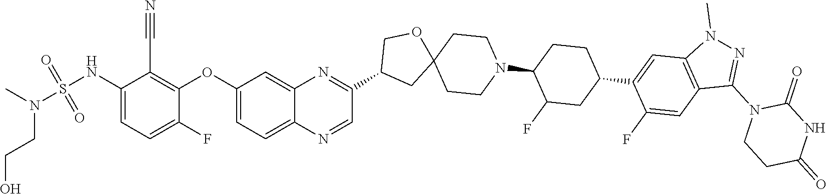 [SMILES: CN(CCO)S(=O)(=O)Nc1ccc(F)c(Oc2ccc3ncc([C@@H]4COC5(CCN([C@H]6CC[C@H](c7cc8c(cc7F)c(N7CCC(=O)NC7=O)nn8C)CC6F)CC5)C4)nc3c2)c1C#N]

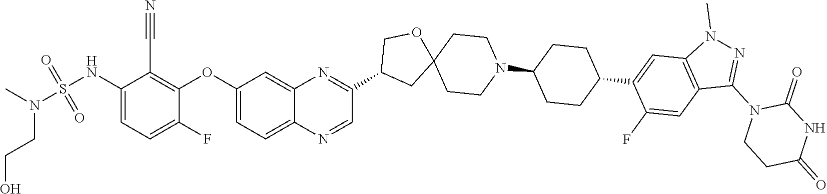 [SMILES: CN(CCO)S(=O)(=O)Nc1ccc(F)c(Oc2ccc3ncc([C@@H]4COC5(CCN([C@H]6CC[C@H](c7cc8c(cc7F)c(N7CCC(=O)NC7=O)nn8C)CC6)CC5)C4)nc3c2)c1C#N]

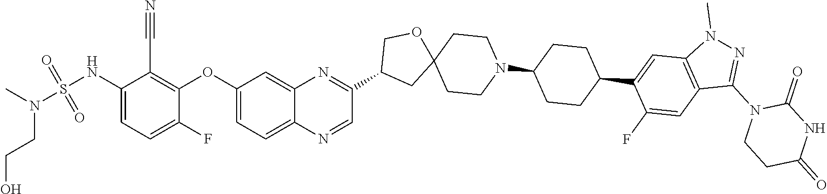 [SMILES: CN(CCO)S(=O)(=O)Nc1ccc(F)c(Oc2ccc3ncc([C@@H]4COC5(CCN([C@H]6CC[C@@H](c7cc8c(cc7F)c(N7CCC(=O)NC7=O)nn8C)CC6)CC5)C4)nc3c2)c1C#N]

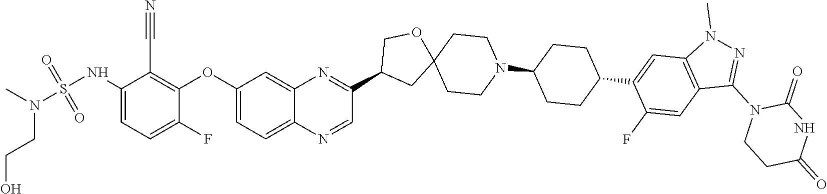 [SMILES: CN(CCO)S(=O)(=O)Nc1ccc(F)c(Oc2ccc3ncc([C@H]4COC5(CCN([C@H]6CC[C@H](c7cc8c(cc7F)c(N7CCC(=O)NC7=O)nn8C)CC6)CC5)C4)nc3c2)c1C#N]

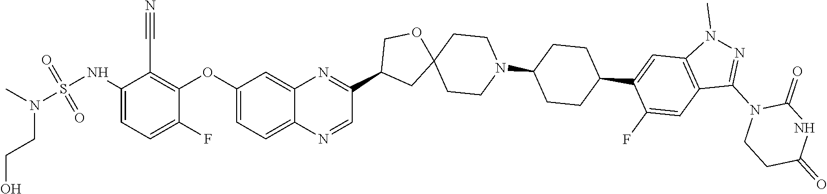 [SMILES: CN(CCO)S(=O)(=O)Nc1ccc(F)c(Oc2ccc3ncc([C@H]4COC5(CCN([C@H]6CC[C@@H](c7cc8c(cc7F)c(N7CCC(=O)NC7=O)nn8C)CC6)CC5)C4)nc3c2)c1C#N]

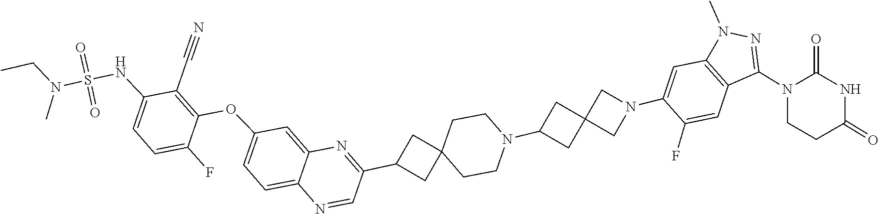 [SMILES: CCN(C)S(=O)(=O)Nc1ccc(F)c(Oc2ccc3ncc(C4CC5(CCN(C6CC7(C6)CN(c6cc8c(cc6F)c(N6CCC(=O)NC6=O)nn8C)C7)CC5)C4)nc3c2)c1C#N]

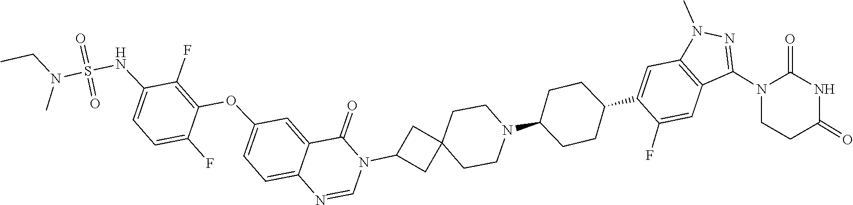 [SMILES: CCN(C)S(=O)(=O)Nc1ccc(F)c(Oc2ccc3ncn(C4CC5(CCN([C@H]6CC[C@H](c7cc8c(cc7F)c(N7CCC(=O)NC7=O)nn8C)CC6)CC5)C4)c(=O)c3c2)c1F]

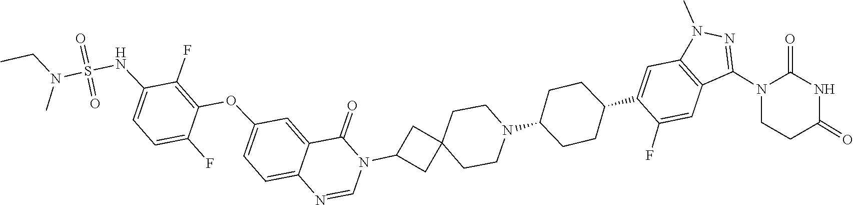 [SMILES: CCN(C)S(=O)(=O)Nc1ccc(F)c(Oc2ccc3ncn(C4CC5(CCN([C@H]6CC[C@@H](c7cc8c(cc7F)c(N7CCC(=O)NC7=O)nn8C)CC6)CC5)C4)c(=O)c3c2)c1F]

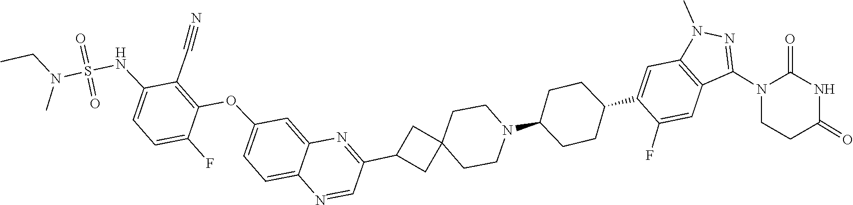 [SMILES: CCN(C)S(=O)(=O)Nc1ccc(F)c(Oc2ccc3ncc(C4CC5(CCN([C@H]6CC[C@H](c7cc8c(cc7F)c(N7CCC(=O)NC7=O)nn8C)CC6)CC5)C4)nc3c2)c1C#N]

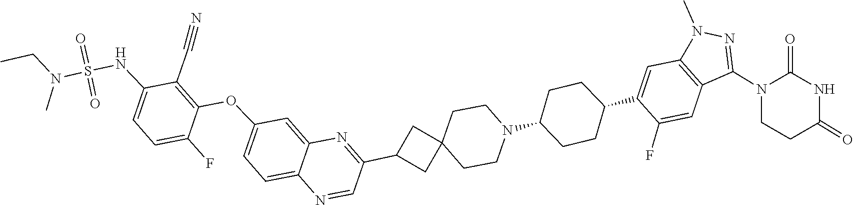 [SMILES: CCN(C)S(=O)(=O)Nc1ccc(F)c(Oc2ccc3ncc(C4CC5(CCN([C@H]6CC[C@@H](c7cc8c(cc7F)c(N7CCC(=O)NC7=O)nn8C)CC6)CC5)C4)nc3c2)c1C#N]

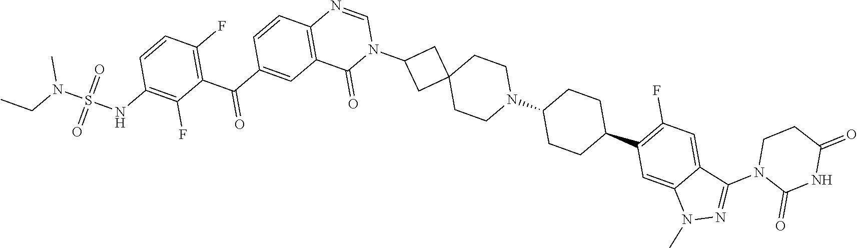 [SMILES: CCN(C)S(=O)(=O)Nc1ccc(F)c(C(=O)c2ccc3ncn(C4CC5(CCN([C@H]6CC[C@H](c7cc8c(cc7F)c(N7CCC(=O)NC7=O)nn8C)CC6)CC5)C4)c(=O)c3c2)c1F]

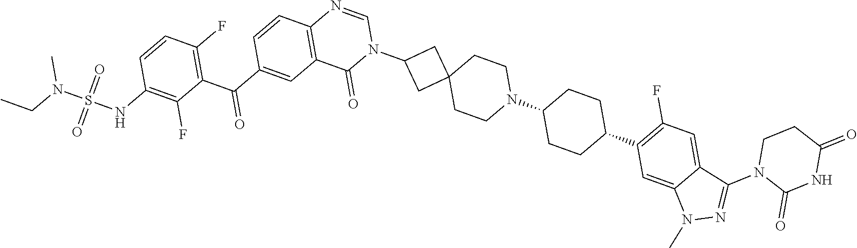 [SMILES: CCN(C)S(=O)(=O)Nc1ccc(F)c(C(=O)c2ccc3ncn(C4CC5(CCN([C@H]6CC[C@@H](c7cc8c(cc7F)c(N7CCC(=O)NC7=O)nn8C)CC6)CC5)C4)c(=O)c3c2)c1F]

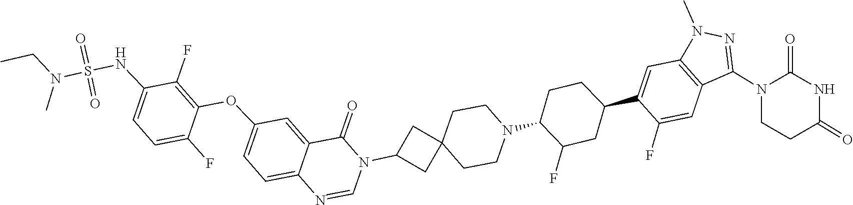 [SMILES: CCN(C)S(=O)(=O)Nc1ccc(F)c(Oc2ccc3ncn(C4CC5(CCN([C@@H]6CC[C@@H](c7cc8c(cc7F)c(N7CCC(=O)NC7=O)nn8C)CC6F)CC5)C4)c(=O)c3c2)c1F]

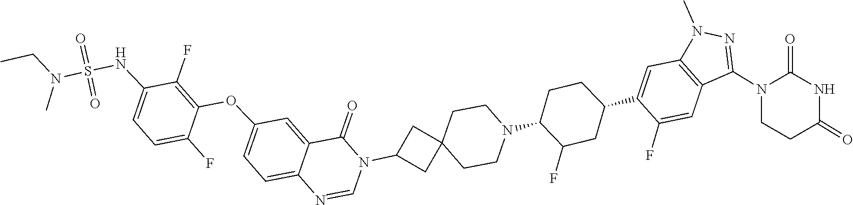 [SMILES: CCN(C)S(=O)(=O)Nc1ccc(F)c(Oc2ccc3ncn(C4CC5(CCN([C@@H]6CC[C@H](c7cc8c(cc7F)c(N7CCC(=O)NC7=O)nn8C)CC6F)CC5)C4)c(=O)c3c2)c1F]

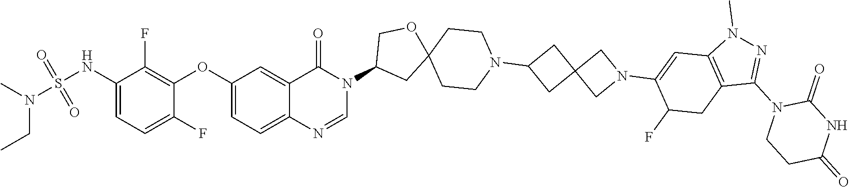 [SMILES: CCN(C)S(=O)(=O)Nc1ccc(F)c(Oc2ccc3ncn([C@H]4COC5(CCN(C6CC7(C6)CN(C6=Cc8c(c(N9CCC(=O)NC9=O)nn8C)CC6F)C7)CC5)C4)c(=O)c3c2)c1F]

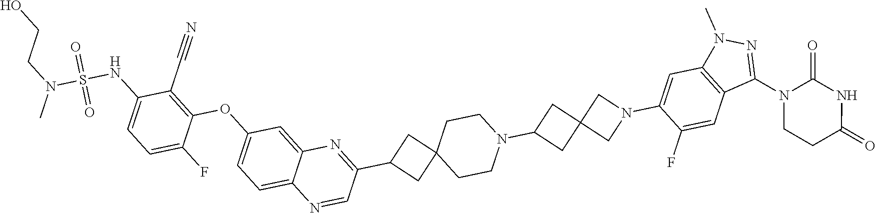 [SMILES: CN(CCO)S(=O)(=O)Nc1ccc(F)c(Oc2ccc3ncc(C4CC5(CCN(C6CC7(C6)CN(c6cc8c(cc6F)c(N6CCC(=O)NC6=O)nn8C)C7)CC5)C4)nc3c2)c1C#N]

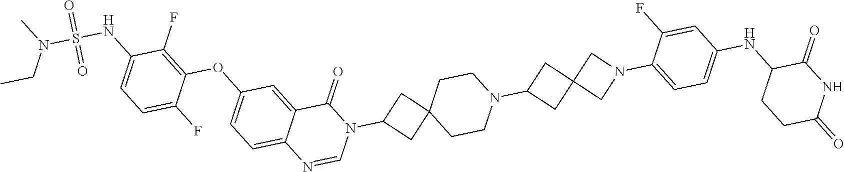 [SMILES: CCN(C)S(=O)(=O)Nc1ccc(F)c(Oc2ccc3ncn(C4CC5(CCN(C6CC7(C6)CN(c6ccc(NC8CCC(=O)NC8=O)cc6F)C7)CC5)C4)c(=O)c3c2)c1F]

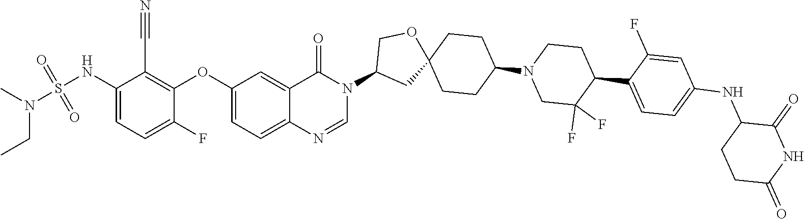 [SMILES: CCN(C)S(=O)(=O)Nc1ccc(F)c(Oc2ccc3ncn([C@H]4CO[C@]5(CC[C@@H](N6CC[C@@H](c7ccc(NC8CCC(=O)NC8=O)cc7F)C(F)(F)C6)CC5)C4)c(=O)c3c2)c1C#N]

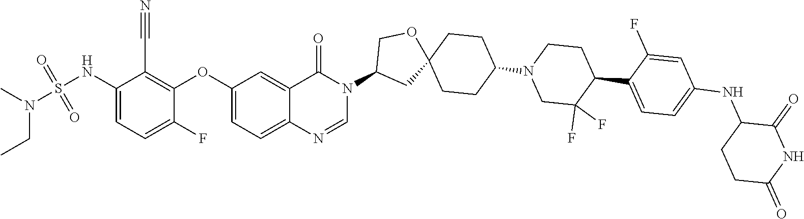 [SMILES: CCN(C)S(=O)(=O)Nc1ccc(F)c(Oc2ccc3ncn([C@H]4CO[C@]5(CC[C@H](N6CC[C@@H](c7ccc(NC8CCC(=O)NC8=O)cc7F)C(F)(F)C6)CC5)C4)c(=O)c3c2)c1C#N]

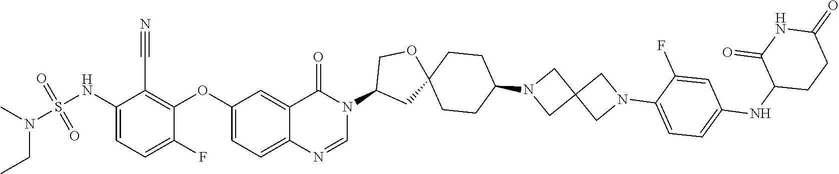 [SMILES: CCN(C)S(=O)(=O)Nc1ccc(F)c(Oc2ccc3ncn([C@H]4CO[C@]5(CC[C@@H](N6CC7(CN(c8ccc(NC9CCC(=O)NC9=O)cc8F)C7)C6)CC5)C4)c(=O)c3c2)c1C#N]

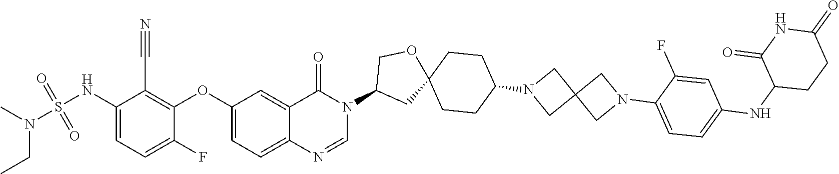 [SMILES: CCN(C)S(=O)(=O)Nc1ccc(F)c(Oc2ccc3ncn([C@H]4CO[C@]5(CC[C@H](N6CC7(CN(c8ccc(NC9CCC(=O)NC9=O)cc8F)C7)C6)CC5)C4)c(=O)c3c2)c1C#N]

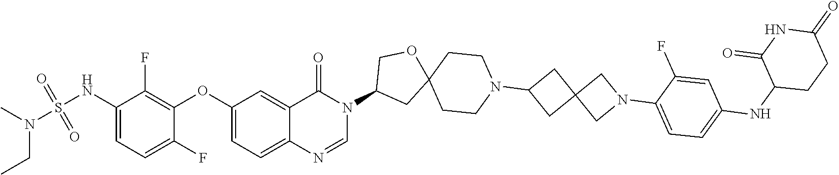 [SMILES: CCN(C)S(=O)(=O)Nc1ccc(F)c(Oc2ccc3ncn([C@H]4COC5(CCN(C6CC7(C6)CN(c6ccc(NC8CCC(=O)NC8=O)cc6F)C7)CC5)C4)c(=O)c3c2)c1F]

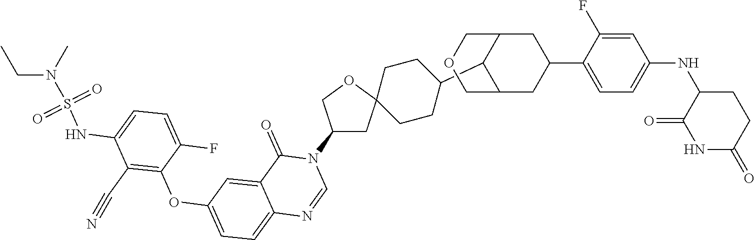 [SMILES: CCN(C)S(=O)(=O)Nc1ccc(F)c(Oc2ccc3ncn([C@H]4COC5(CCC(C6C7COCC6CC(c6ccc(NC8CCC(=O)NC8=O)cc6F)C7)CC5)C4)c(=O)c3c2)c1C#N]

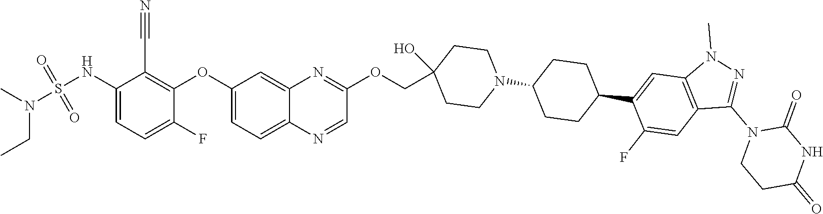 [SMILES: CCN(C)S(=O)(=O)Nc1ccc(F)c(Oc2ccc3ncc(OCC4(O)CCN([C@H]5CC[C@H](c6cc7c(cc6F)c(N6CCC(=O)NC6=O)nn7C)CC5)CC4)nc3c2)c1C#N]

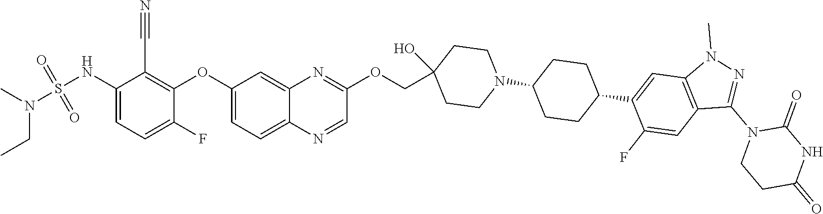 [SMILES: CCN(C)S(=O)(=O)Nc1ccc(F)c(Oc2ccc3ncc(OCC4(O)CCN([C@H]5CC[C@@H](c6cc7c(cc6F)c(N6CCC(=O)NC6=O)nn7C)CC5)CC4)nc3c2)c1C#N]

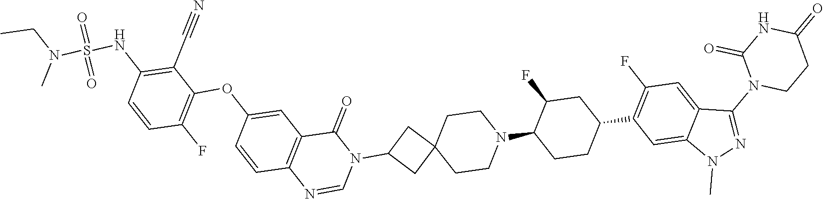 [SMILES: CCN(C)S(=O)(=O)Nc1ccc(F)c(Oc2ccc3ncn(C4CC5(CCN([C@@H]6CC[C@@H](c7cc8c(cc7F)c(N7CCC(=O)NC7=O)nn8C)C[C@@H]6F)CC5)C4)c(=O)c3c2)c1C#N]

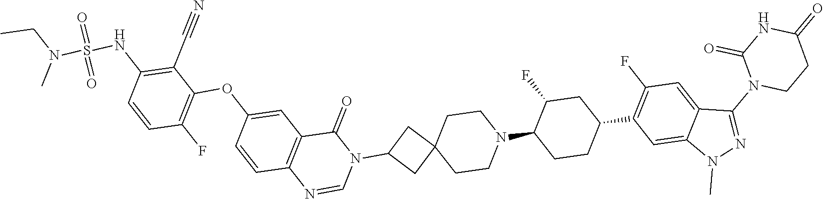 [SMILES: CCN(C)S(=O)(=O)Nc1ccc(F)c(Oc2ccc3ncn(C4CC5(CCN([C@@H]6CC[C@@H](c7cc8c(cc7F)c(N7CCC(=O)NC7=O)nn8C)C[C@H]6F)CC5)C4)c(=O)c3c2)c1C#N]

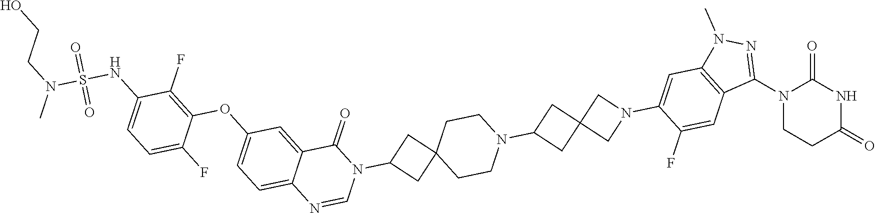 [SMILES: CN(CCO)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1F]

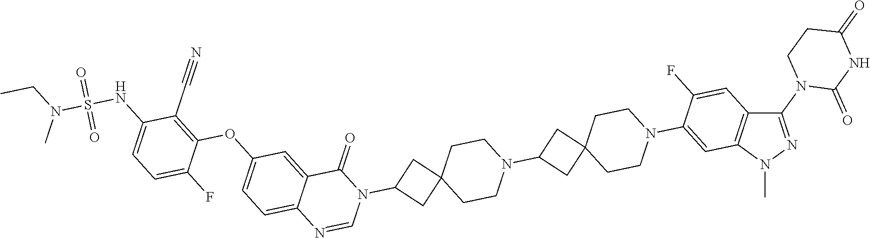 [SMILES: CCN(C)S(=O)(=O)Nc1ccc(F)c(Oc2ccc3ncn(C4CC5(CCN(C6CC7(CCN(c8cc9c(cc8F)c(N8CCC(=O)NC8=O)nn9C)CC7)C6)CC5)C4)c(=O)c3c2)c1C#N]

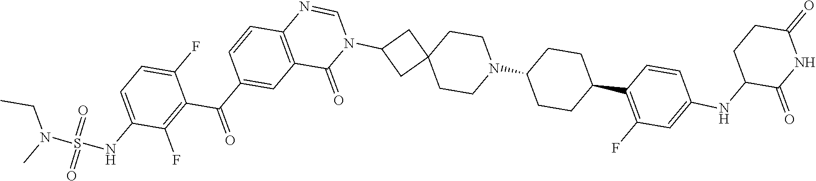 [SMILES: CCN(C)S(=O)(=O)Nc1ccc(F)c(C(=O)c2ccc3ncn(C4CC5(CCN([C@H]6CC[C@H](c7ccc(NC8CCC(=O)NC8=O)cc7F)CC6)CC5)C4)c(=O)c3c2)c1F]

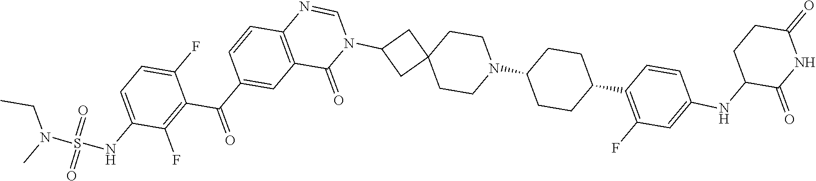 [SMILES: CCN(C)S(=O)(=O)Nc1ccc(F)c(C(=O)c2ccc3ncn(C4CC5(CCN([C@H]6CC[C@@H](c7ccc(NC8CCC(=O)NC8=O)cc7F)CC6)CC5)C4)c(=O)c3c2)c1F]

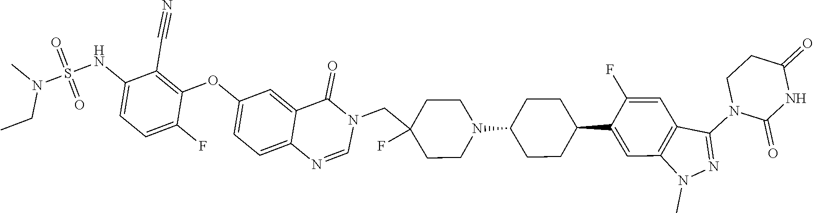 [SMILES: CCN(C)S(=O)(=O)Nc1ccc(F)c(Oc2ccc3ncn(CC4(F)CCN([C@H]5CC[C@H](c6cc7c(cc6F)c(N6CCC(=O)NC6=O)nn7C)CC5)CC4)c(=O)c3c2)c1C#N]

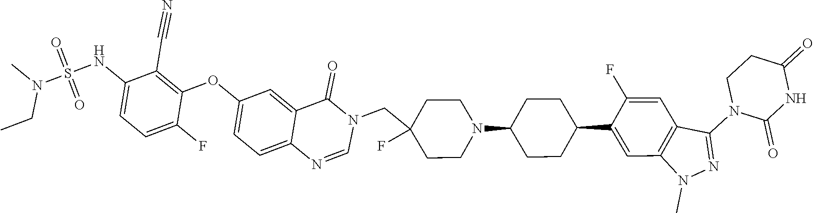 [SMILES: CCN(C)S(=O)(=O)Nc1ccc(F)c(Oc2ccc3ncn(CC4(F)CCN([C@H]5CC[C@@H](c6cc7c(cc6F)c(N6CCC(=O)NC6=O)nn7C)CC5)CC4)c(=O)c3c2)c1C#N]

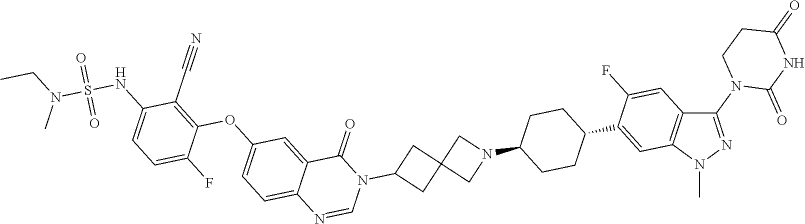 [SMILES: CCN(C)S(=O)(=O)Nc1ccc(F)c(Oc2ccc3ncn(C4CC5(C4)CN([C@H]4CC[C@H](c6cc7c(cc6F)c(N6CCC(=O)NC6=O)nn7C)CC4)C5)c(=O)c3c2)c1C#N]

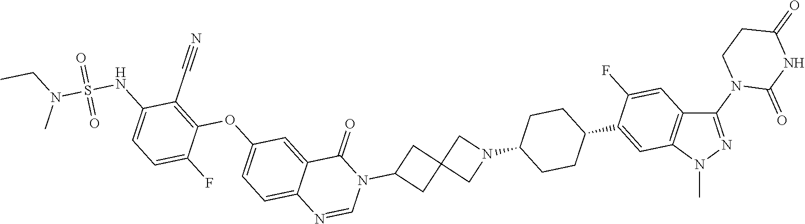 [SMILES: CCN(C)S(=O)(=O)Nc1ccc(F)c(Oc2ccc3ncn(C4CC5(C4)CN([C@H]4CC[C@@H](c6cc7c(cc6F)c(N6CCC(=O)NC6=O)nn7C)CC4)C5)c(=O)c3c2)c1C#N]

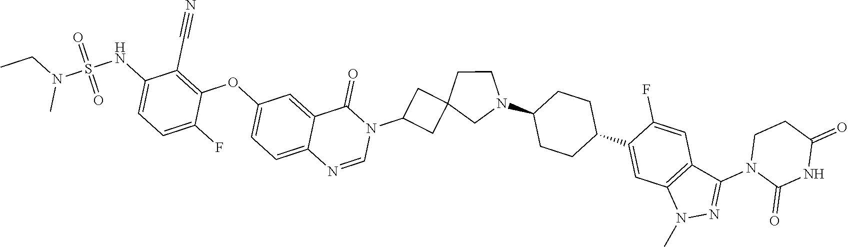 [SMILES: CCN(C)S(=O)(=O)Nc1ccc(F)c(Oc2ccc3ncn(C4CC5(CCN([C@H]6CC[C@H](c7cc8c(cc7F)c(N7CCC(=O)NC7=O)nn8C)CC6)C5)C4)c(=O)c3c2)c1C#N]

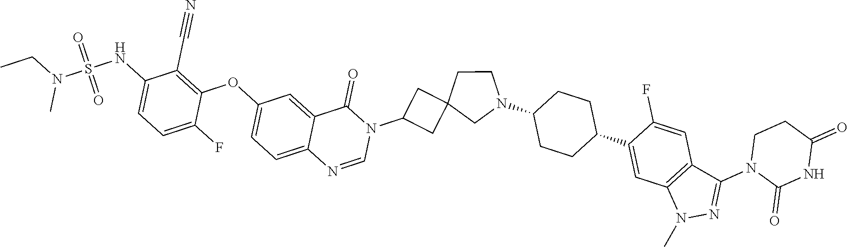 [SMILES: CCN(C)S(=O)(=O)Nc1ccc(F)c(Oc2ccc3ncn(C4CC5(CCN([C@H]6CC[C@@H](c7cc8c(cc7F)c(N7CCC(=O)NC7=O)nn8C)CC6)C5)C4)c(=O)c3c2)c1C#N]

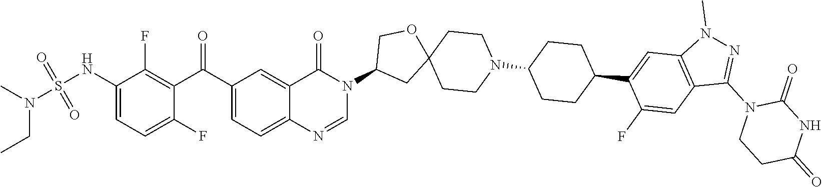 [SMILES: CCN(C)S(=O)(=O)Nc1ccc(F)c(C(=O)c2ccc3ncn([C@H]4COC5(CCN([C@H]6CC[C@H](c7cc8c(cc7F)c(N7CCC(=O)NC7=O)nn8C)CC6)CC5)C4)c(=O)c3c2)c1F]

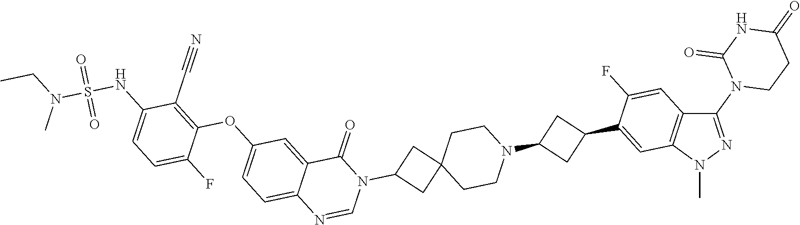 [SMILES: CCN(C)S(=O)(=O)Nc1ccc(F)c(Oc2ccc3ncn(C4CC5(CCN([C@H]6C[C@@H](c7cc8c(cc7F)c(N7CCC(=O)NC7=O)nn8C)C6)CC5)C4)c(=O)c3c2)c1C#N]

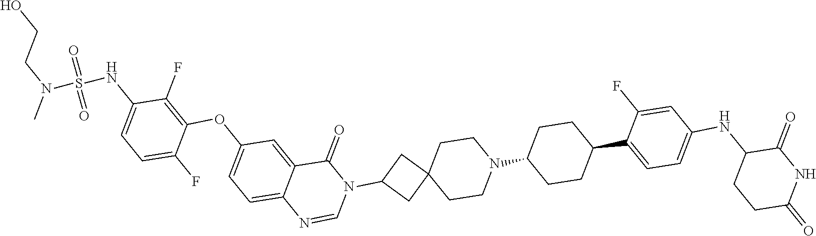 [SMILES: CN(CCO)S(=O)(=O)Nc1ccc(F)c(Oc2ccc3ncn(C4CC5(CCN([C@H]6CC[C@H](c7ccc(NC8CCC(=O)NC8=O)cc7F)CC6)CC5)C4)c(=O)c3c2)c1F]

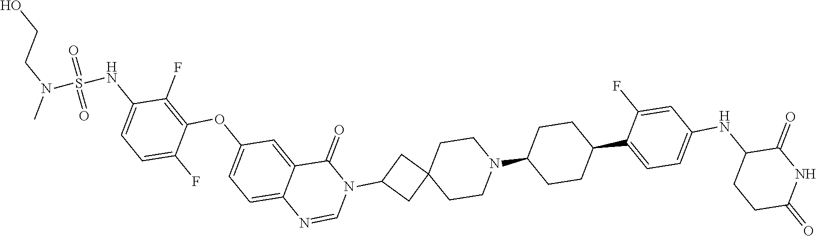 [SMILES: CN(CCO)S(=O)(=O)Nc1ccc(F)c(Oc2ccc3ncn(C4CC5(CCN([C@H]6CC[C@@H](c7ccc(NC8CCC(=O)NC8=O)cc7F)CC6)CC5)C4)c(=O)c3c2)c1F]

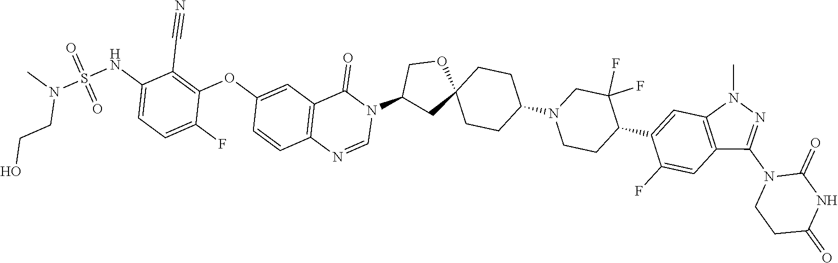 [SMILES: CN(CCO)S(=O)(=O)Nc1ccc(F)c(Oc2ccc3ncn([C@H]4CO[C@]5(CC[C@@H](N6CC[C@@H](c7cc8c(cc7F)c(N7CCC(=O)NC7=O)nn8C)C(F)(F)C6)CC5)C4)c(=O)c3c2)c1C#N]

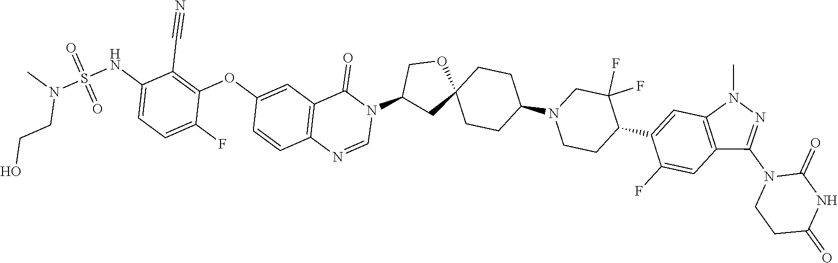 [SMILES: CN(CCO)S(=O)(=O)Nc1ccc(F)c(Oc2ccc3ncn([C@H]4CO[C@]5(CC[C@H](N6CC[C@@H](c7cc8c(cc7F)c(N7CCC(=O)NC7=O)nn8C)C(F)(F)C6)CC5)C4)c(=O)c3c2)c1C#N]

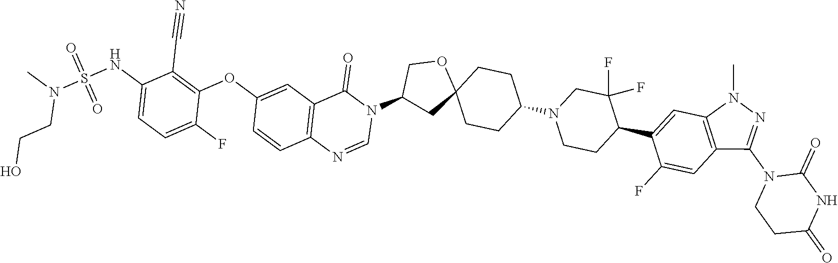 [SMILES: CN(CCO)S(=O)(=O)Nc1ccc(F)c(Oc2ccc3ncn([C@H]4CO[C@]5(CC[C@@H](N6CC[C@H](c7cc8c(cc7F)c(N7CCC(=O)NC7=O)nn8C)C(F)(F)C6)CC5)C4)c(=O)c3c2)c1C#N]

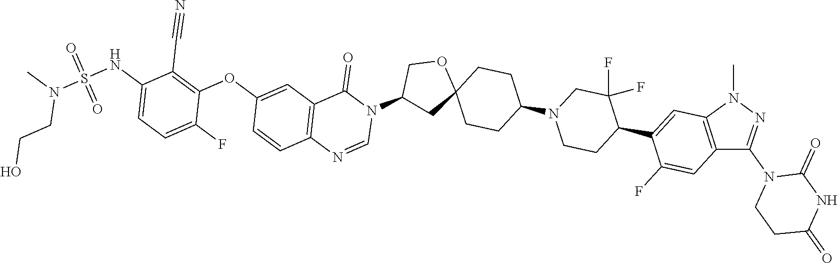 [SMILES: CN(CCO)S(=O)(=O)Nc1ccc(F)c(Oc2ccc3ncn([C@H]4CO[C@]5(CC[C@H](N6CC[C@H](c7cc8c(cc7F)c(N7CCC(=O)NC7=O)nn8C)C(F)(F)C6)CC5)C4)c(=O)c3c2)c1C#N]

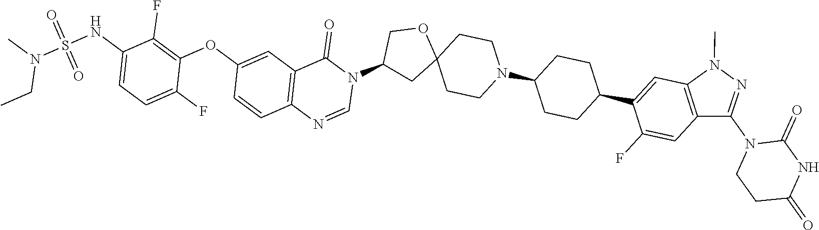 [SMILES: CCN(C)S(=O)(=O)Nc1ccc(F)c(Oc2ccc3ncn([C@H]4COC5(CCN([C@H]6CC[C@@H](c7cc8c(cc7F)c(N7CCC(=O)NC7=O)nn8C)CC6)CC5)C4)c(=O)c3c2)c1F]

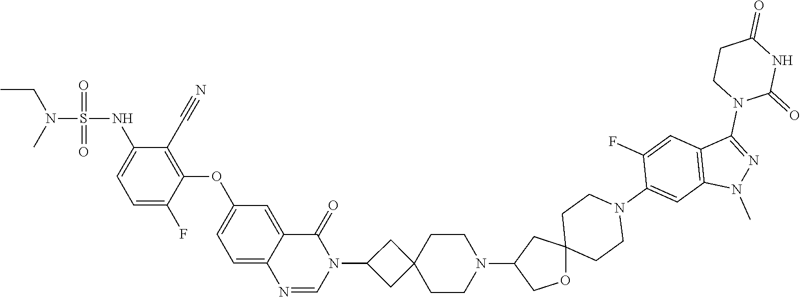 [SMILES: CCN(C)S(=O)(=O)Nc1ccc(F)c(Oc2ccc3ncn(C4CC5(CCN(C6COC7(CCN(c8cc9c(cc8F)c(N8CCC(=O)NC8=O)nn9C)CC7)C6)CC5)C4)c(=O)c3c2)c1C#N]

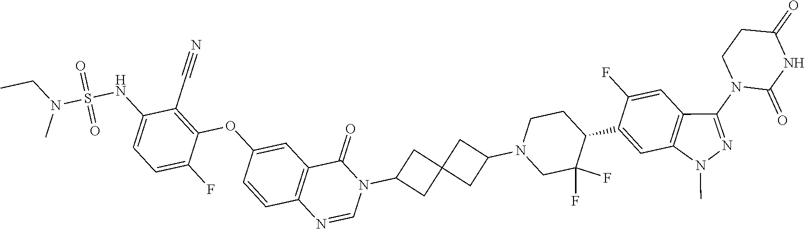 [SMILES: CCN(C)S(=O)(=O)Nc1ccc(F)c(Oc2ccc3ncn(C4CC5(CC(N6CC[C@H](c7cc8c(cc7F)c(N7CCC(=O)NC7=O)nn8C)C(F)(F)C6)C5)C4)c(=O)c3c2)c1C#N]

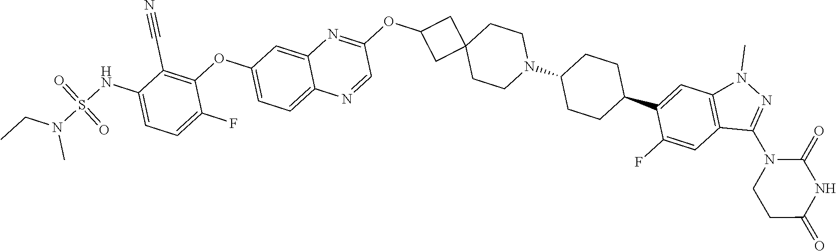 [SMILES: CCN(C)S(=O)(=O)Nc1ccc(F)c(Oc2ccc3ncc(OC4CC5(CCN([C@H]6CC[C@H](c7cc8c(cc7F)c(N7CCC(=O)NC7=O)nn8C)CC6)CC5)C4)nc3c2)c1C#N]

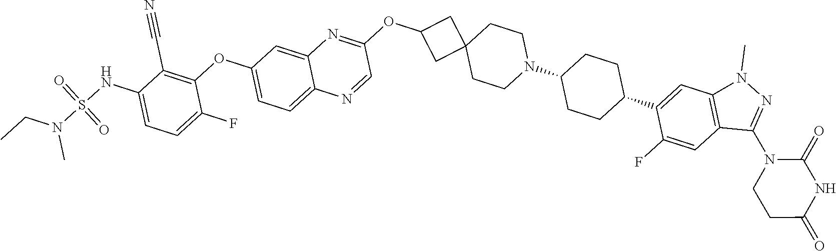 [SMILES: CCN(C)S(=O)(=O)Nc1ccc(F)c(Oc2ccc3ncc(OC4CC5(CCN([C@H]6CC[C@@H](c7cc8c(cc7F)c(N7CCC(=O)NC7=O)nn8C)CC6)CC5)C4)nc3c2)c1C#N]

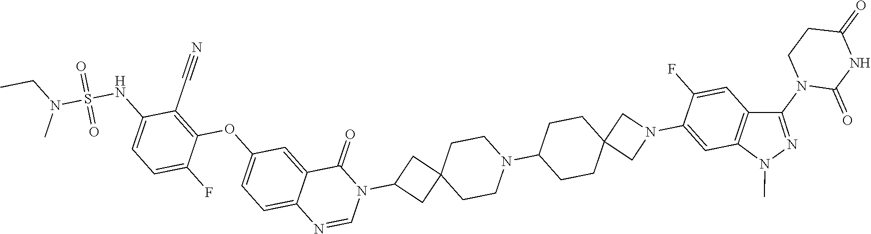 [SMILES: CCN(C)S(=O)(=O)Nc1ccc(F)c(Oc2ccc3ncn(C4CC5(CCN(C6CCC7(CC6)CN(c6cc8c(cc6F)c(N6CCC(=O)NC6=O)nn8C)C7)CC5)C4)c(=O)c3c2)c1C#N]

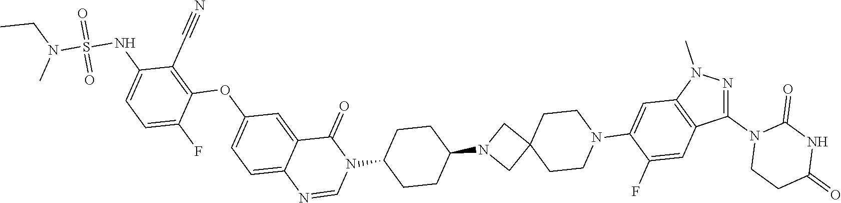 [SMILES: CCN(C)S(=O)(=O)Nc1ccc(F)c(Oc2ccc3ncn([C@H]4CC[C@H](N5CC6(CCN(c7cc8c(cc7F)c(N7CCC(=O)NC7=O)nn8C)CC6)C5)CC4)c(=O)c3c2)c1C#N]

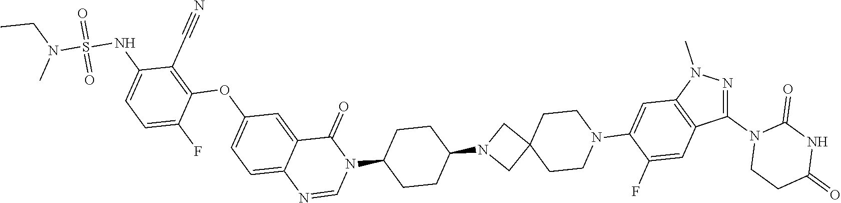 [SMILES: CCN(C)S(=O)(=O)Nc1ccc(F)c(Oc2ccc3ncn([C@H]4CC[C@@H](N5CC6(CCN(c7cc8c(cc7F)c(N7CCC(=O)NC7=O)nn8C)CC6)C5)CC4)c(=O)c3c2)c1C#N]

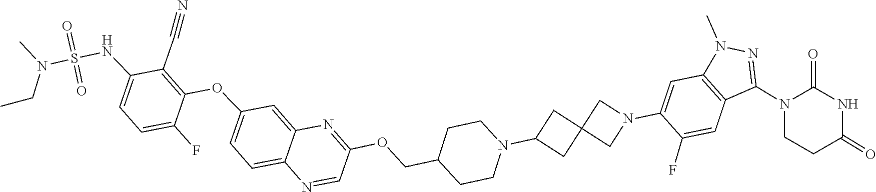 [SMILES: CCN(C)S(=O)(=O)Nc1ccc(F)c(Oc2ccc3ncc(OCC4CCN(C5CC6(C5)CN(c5cc7c(cc5F)c(N5CCC(=O)NC5=O)nn7C)C6)CC4)nc3c2)c1C#N]

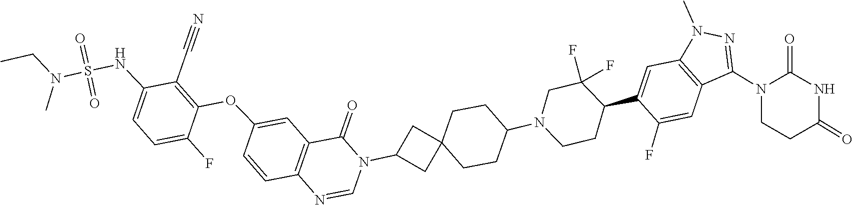 [SMILES: CCN(C)S(=O)(=O)Nc1ccc(F)c(Oc2ccc3ncn(C4CC5(CCC(N6CC[C@H](c7cc8c(cc7F)c(N7CCC(=O)NC7=O)nn8C)C(F)(F)C6)CC5)C4)c(=O)c3c2)c1C#N]

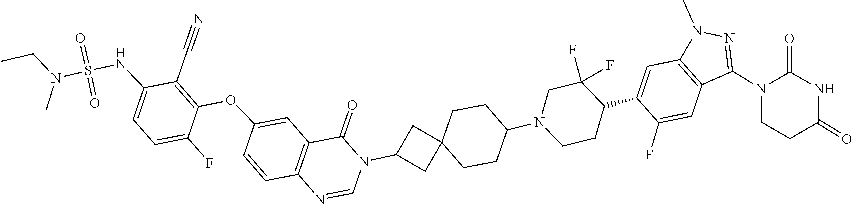

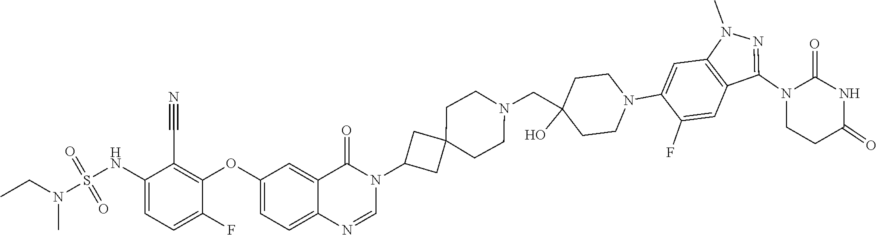 [SMILES: CCN(C)S(=O)(=O)Nc1ccc(F)c(Oc2ccc3ncn(C4CC5(CCN(CC6(O)CCN(c7cc8c(cc7F)c(N7CCC(=O)NC7=O)nn8C)CC6)CC5)C4)c(=O)c3c2)c1C#N]

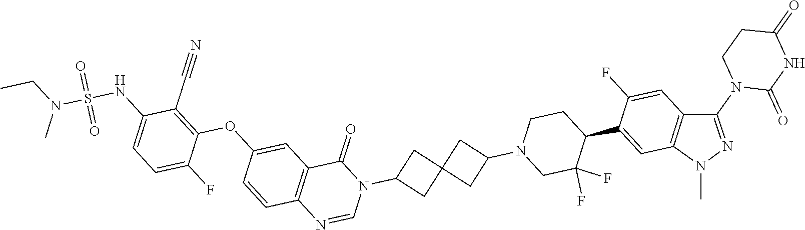 [SMILES: CCN(C)S(=O)(=O)Nc1ccc(F)c(Oc2ccc3ncn(C4CC5(CC(N6CC[C@@H](c7cc8c(cc7F)c(N7CCC(=O)NC7=O)nn8C)C(F)(F)C6)C5)C4)c(=O)c3c2)c1C#N]

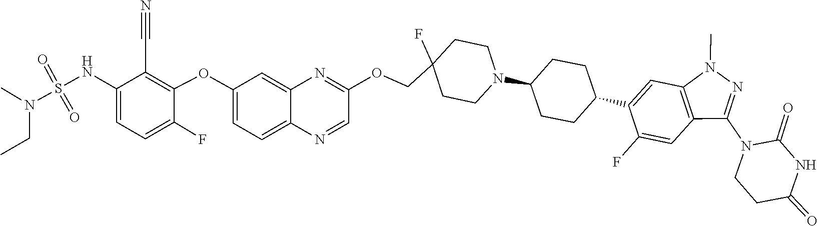 [SMILES: CCN(C)S(=O)(=O)Nc1ccc(F)c(Oc2ccc3ncc(OCC4(F)CCN([C@H]5CC[C@H](c6cc7c(cc6F)c(N6CCC(=O)NC6=O)nn7C)CC5)CC4)nc3c2)c1C#N]

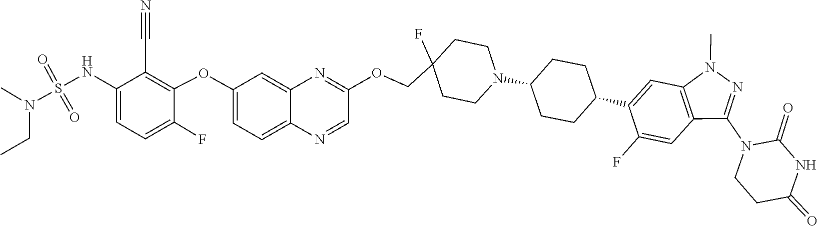 [SMILES: CCN(C)S(=O)(=O)Nc1ccc(F)c(Oc2ccc3ncc(OCC4(F)CCN([C@H]5CC[C@@H](c6cc7c(cc6F)c(N6CCC(=O)NC6=O)nn7C)CC5)CC4)nc3c2)c1C#N]

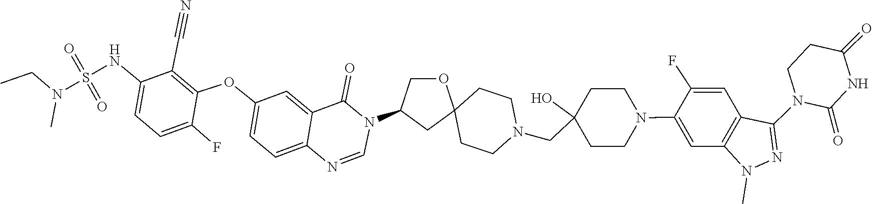 [SMILES: CCN(C)S(=O)(=O)Nc1ccc(F)c(Oc2ccc3ncn([C@H]4COC5(CCN(CC6(O)CCN(c7cc8c(cc7F)c(N7CCC(=O)NC7=O)nn8C)CC6)CC5)C4)c(=O)c3c2)c1C#N]

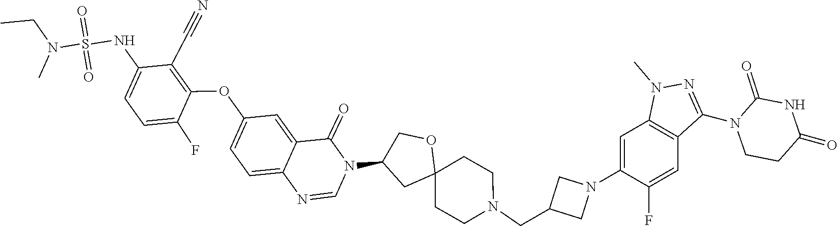 [SMILES: CCN(C)S(=O)(=O)Nc1ccc(F)c(Oc2ccc3ncn([C@H]4COC5(CCN(CC6CN(c7cc8c(cc7F)c(N7CCC(=O)NC7=O)nn8C)C6)CC5)C4)c(=O)c3c2)c1C#N]

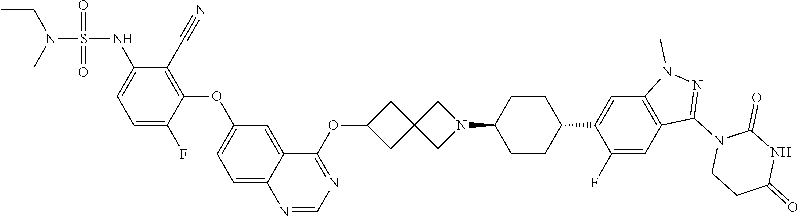 [SMILES: CCN(C)S(=O)(=O)Nc1ccc(F)c(Oc2ccc3ncnc(OC4CC5(C4)CN([C@H]4CC[C@H](c6cc7c(cc6F)c(N6CCC(=O)NC6=O)nn7C)CC4)C5)c3c2)c1C#N]

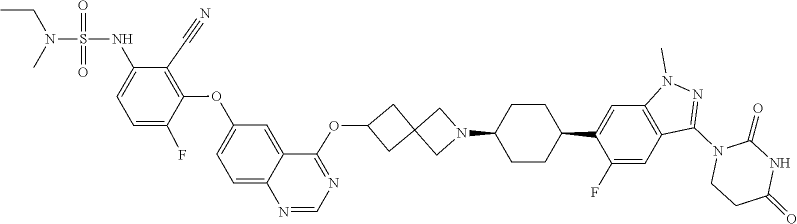 [SMILES: CCN(C)S(=O)(=O)Nc1ccc(F)c(Oc2ccc3ncnc(OC4CC5(C4)CN([C@H]4CC[C@@H](c6cc7c(cc6F)c(N6CCC(=O)NC6=O)nn7C)CC4)C5)c3c2)c1C#N]

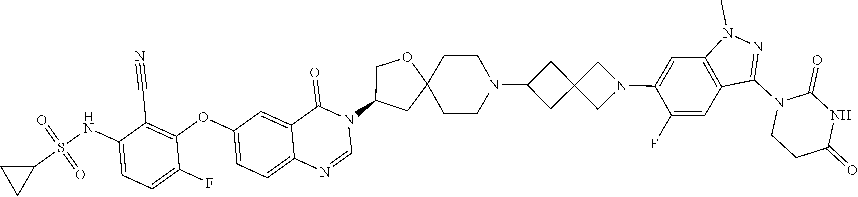 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CC9)c8C#N)cc7c5=O)CO6)C4)C3)cc21]

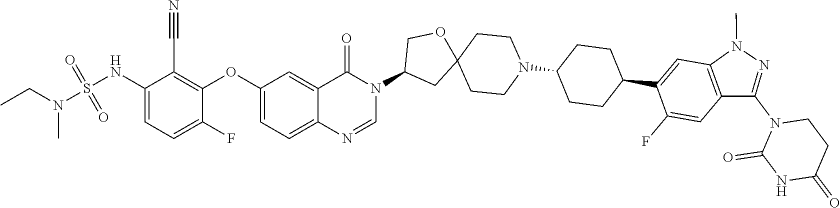 [SMILES: CCN(C)S(=O)(=O)Nc1ccc(F)c(Oc2ccc3ncn([C@H]4COC5(CCN([C@H]6CC[C@H](c7cc8c(cc7F)c(N7CCC(=O)NC7=O)nn8C)CC6)CC5)C4)c(=O)c3c2)c1C#N]

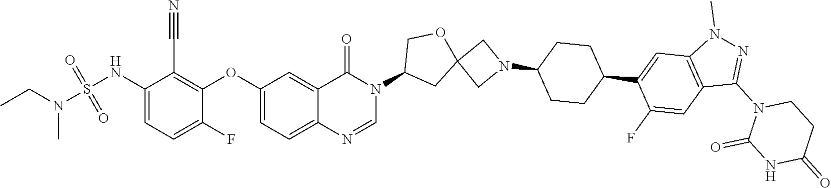 [SMILES: CCN(C)S(=O)(=O)Nc1ccc(F)c(Oc2ccc3ncn([C@H]4COC5(C4)CN([C@H]4CC[C@@H](c6cc7c(cc6F)c(N6CCC(=O)NC6=O)nn7C)CC4)C5)c(=O)c3c2)c1C#N]

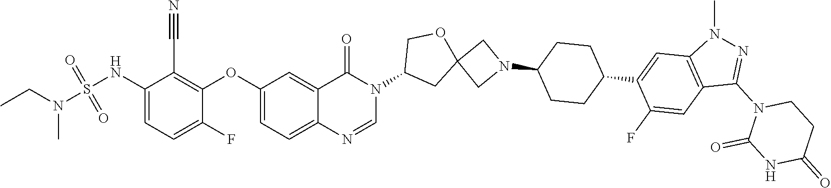 [SMILES: CCN(C)S(=O)(=O)Nc1ccc(F)c(Oc2ccc3ncn([C@@H]4COC5(C4)CN([C@H]4CC[C@H](c6cc7c(cc6F)c(N6CCC(=O)NC6=O)nn7C)CC4)C5)c(=O)c3c2)c1C#N]

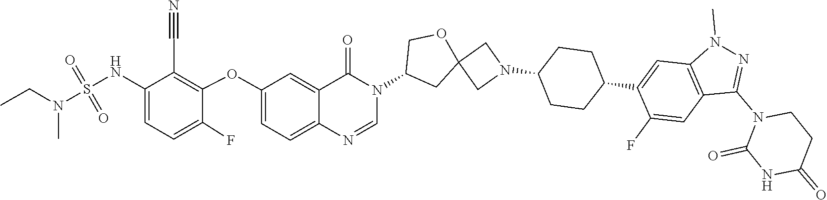 [SMILES: CCN(C)S(=O)(=O)Nc1ccc(F)c(Oc2ccc3ncn([C@@H]4COC5(C4)CN([C@H]4CC[C@@H](c6cc7c(cc6F)c(N6CCC(=O)NC6=O)nn7C)CC4)C5)c(=O)c3c2)c1C#N]

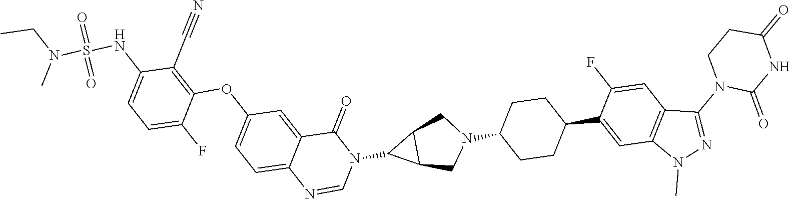

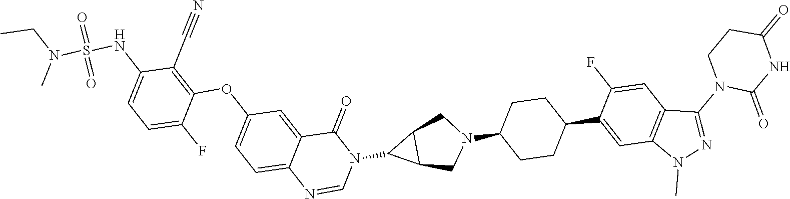

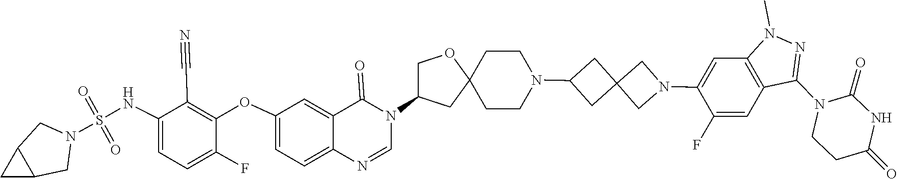 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC%10CC%10C9)c8C#N)cc7c5=O)CO6)C4)C3)cc21]

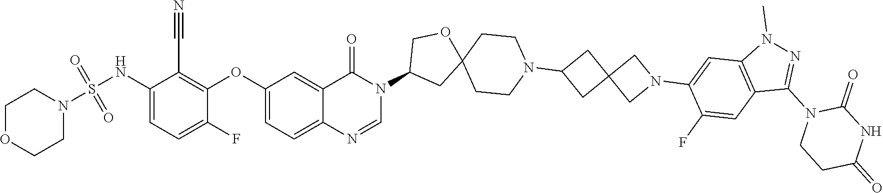 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CCOCC9)c8C#N)cc7c5=O)CO6)C4)C3)cc21]

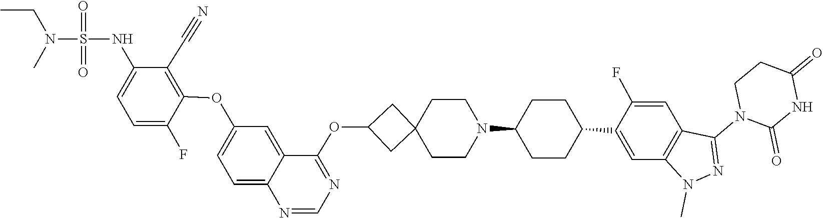 [SMILES: CCN(C)S(=O)(=O)Nc1ccc(F)c(Oc2ccc3ncnc(OC4CC5(CCN([C@H]6CC[C@H](c7cc8c(cc7F)c(N7CCC(=O)NC7=O)nn8C)CC6)CC5)C4)c3c2)c1C#N]

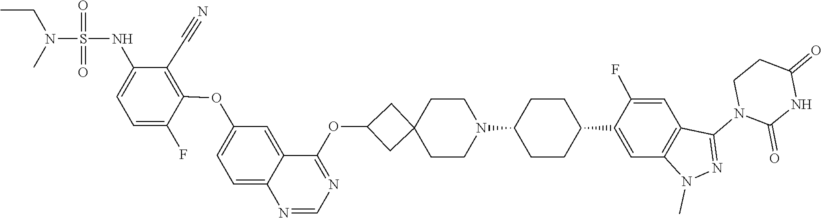 [SMILES: CCN(C)S(=O)(=O)Nc1ccc(F)c(Oc2ccc3ncnc(OC4CC5(CCN([C@H]6CC[C@@H](c7cc8c(cc7F)c(N7CCC(=O)NC7=O)nn8C)CC6)CC5)C4)c3c2)c1C#N]

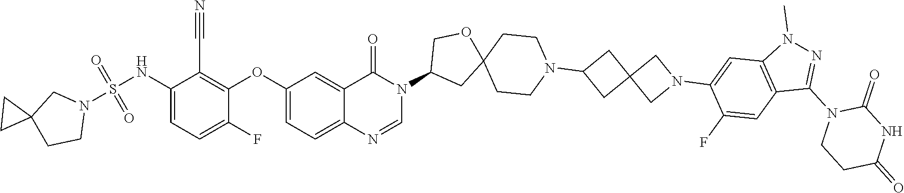 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CCC%10(CC%10)C9)c8C#N)cc7c5=O)CO6)C4)C3)cc21]

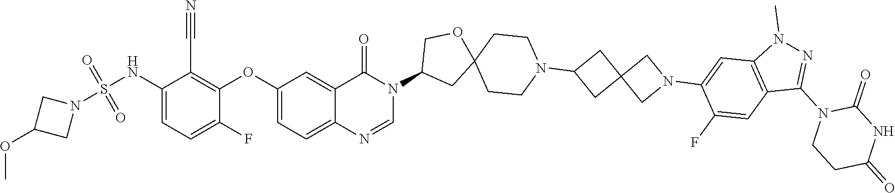 [SMILES: COC1CN(S(=O)(=O)Nc2ccc(F)c(Oc3ccc4ncn([C@H]5COC6(CCN(C7CC8(C7)CN(c7cc9c(cc7F)c(N7CCC(=O)NC7=O)nn9C)C8)CC6)C5)c(=O)c4c3)c2C#N)C1]

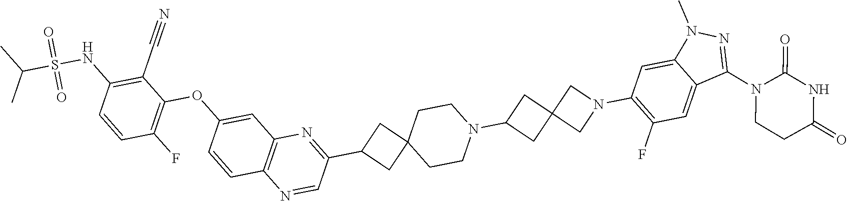 [SMILES: CC(C)S(=O)(=O)Nc1ccc(F)c(Oc2ccc3ncc(C4CC5(CCN(C6CC7(C6)CN(c6cc8c(cc6F)c(N6CCC(=O)NC6=O)nn8C)C7)CC5)C4)nc3c2)c1C#N]

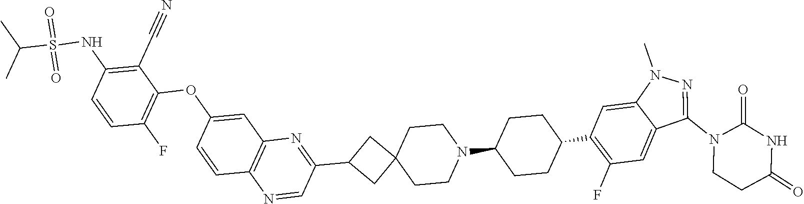 [SMILES: CC(C)S(=O)(=O)Nc1ccc(F)c(Oc2ccc3ncc(C4CC5(CCN([C@H]6CC[C@H](c7cc8c(cc7F)c(N7CCC(=O)NC7=O)nn8C)CC6)CC5)C4)nc3c2)c1C#N]

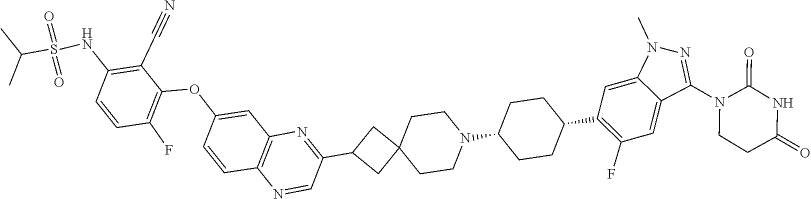 [SMILES: CC(C)S(=O)(=O)Nc1ccc(F)c(Oc2ccc3ncc(C4CC5(CCN([C@H]6CC[C@@H](c7cc8c(cc7F)c(N7CCC(=O)NC7=O)nn8C)CC6)CC5)C4)nc3c2)c1C#N]

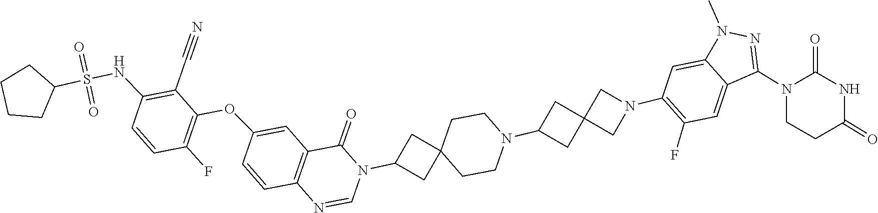 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)C9CCCC9)c8C#N)cc7c5=O)C6)C4)C3)cc21]

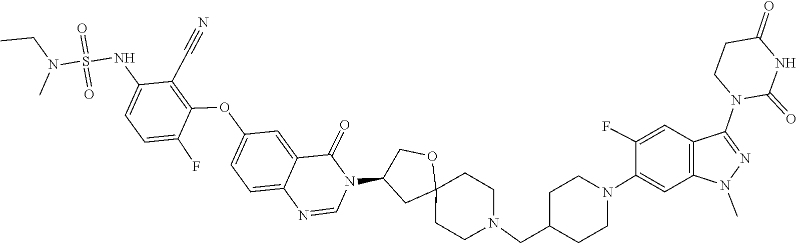 [SMILES: CCN(C)S(=O)(=O)Nc1ccc(F)c(Oc2ccc3ncn([C@H]4COC5(CCN(CC6CCN(c7cc8c(cc7F)c(N7CCC(=O)NC7=O)nn8C)CC6)CC5)C4)c(=O)c3c2)c1C#N]

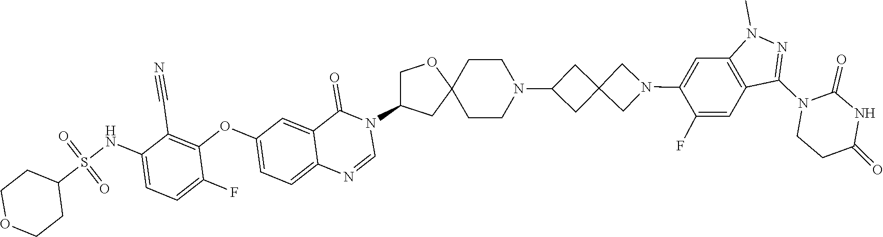 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CCOCC9)c8C#N)cc7c5=O)CO6)C4)C3)cc21]

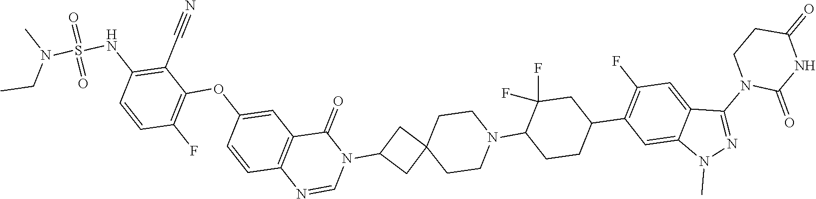 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6(F)F)CC5)C4)c(=O)c3c2)c1C#N]

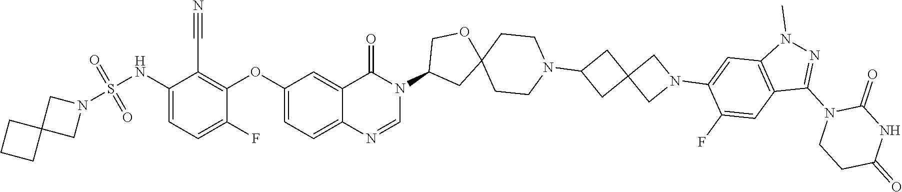 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC%10(CCC%10)C9)c8C#N)cc7c5=O)CO6)C4)C3)cc21]

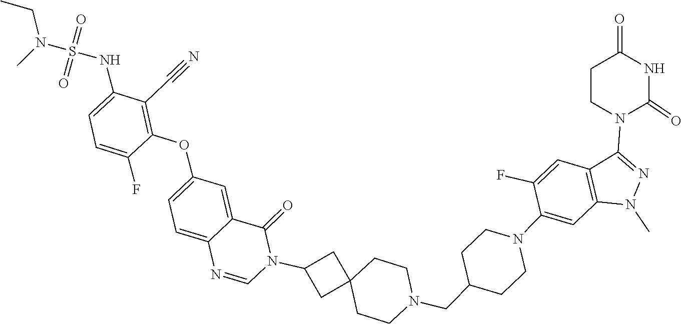 [SMILES: CCN(C)S(=O)(=O)Nc1ccc(F)c(Oc2ccc3ncn(C4CC5(CCN(CC6CCN(c7cc8c(cc7F)c(N7CCC(=O)NC7=O)nn8C)CC6)CC5)C4)c(=O)c3c2)c1C#N]

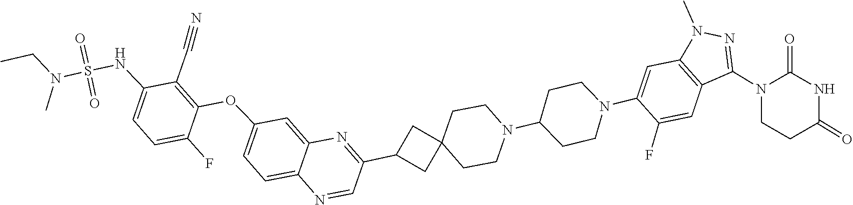 [SMILES: CCN(C)S(=O)(=O)Nc1ccc(F)c(Oc2ccc3ncc(C4CC5(CCN(C6CCN(c7cc8c(cc7F)c(N7CCC(=O)NC7=O)nn8C)CC6)CC5)C4)nc3c2)c1C#N]

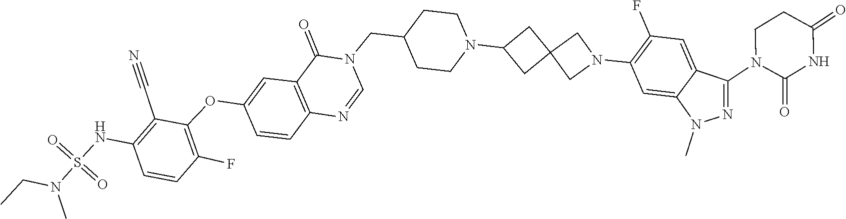 [SMILES: CCN(C)S(=O)(=O)Nc1ccc(F)c(Oc2ccc3ncn(CC4CCN(C5CC6(C5)CN(c5cc7c(cc5F)c(N5CCC(=O)NC5=O)nn7C)C6)CC4)c(=O)c3c2)c1C#N]

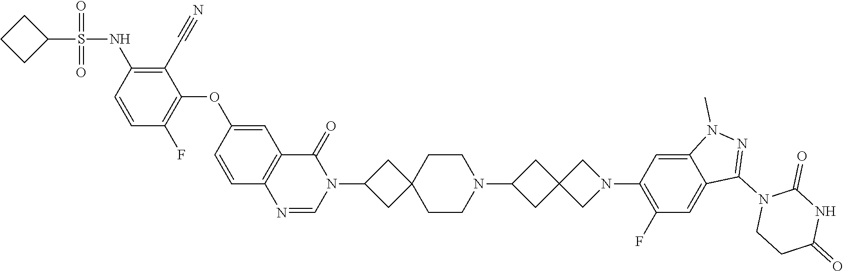 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)C9CCC9)c8C#N)cc7c5=O)C6)C4)C3)cc21]

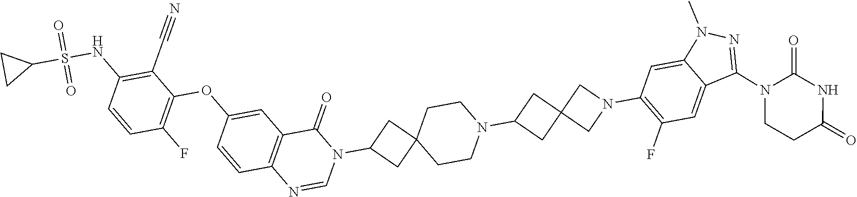 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)C9CC9)c8C#N)cc7c5=O)C6)C4)C3)cc21]

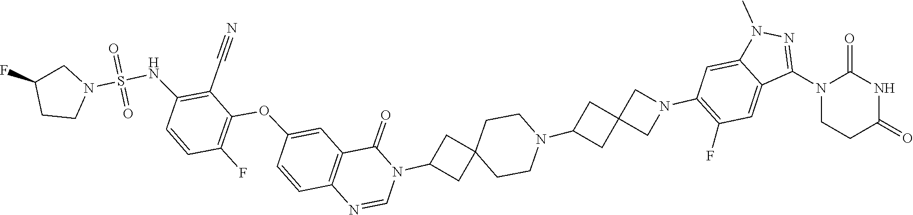 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9CC[C@@H](F)C9)c8C#N)cc7c5=O)C6)C4)C3)cc21]

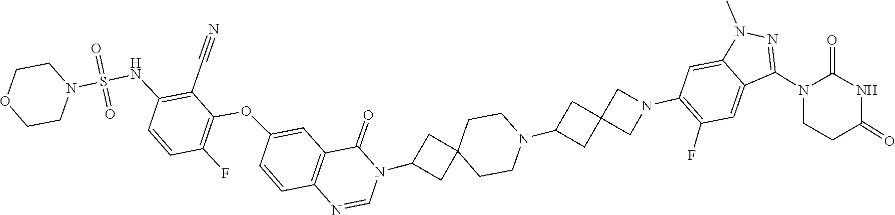 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9CCOCC9)c8C#N)cc7c5=O)C6)C4)C3)cc21]

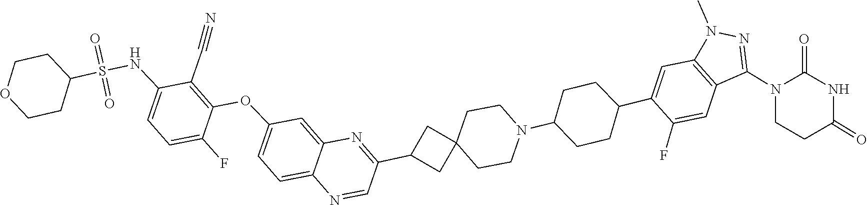 [SMILES: Cn1nc(N2CCC(=O)NC2=O)c2cc(F)c(C3CCC(N4CCC5(CC4)CC(c4cnc6ccc(Oc7c(F)ccc(NS(=O)(=O)C8CCOCC8)c7C#N)cc6n4)C5)CC3)cc21]

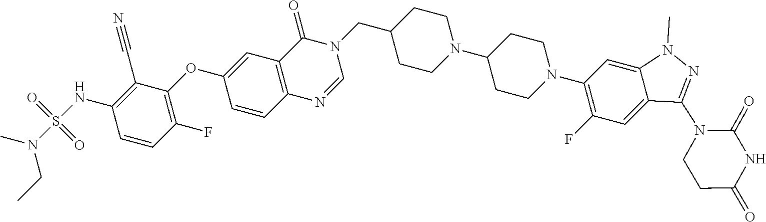 [SMILES: CCN(C)S(=O)(=O)Nc1ccc(F)c(Oc2ccc3ncn(CC4CCN(C5CCN(c6cc7c(cc6F)c(N6CCC(=O)NC6=O)nn7C)CC5)CC4)c(=O)c3c2)c1C#N]

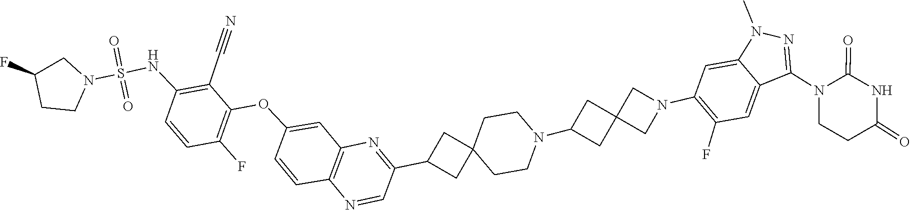 [SMILES: Cn1nc(N2CCC(=O)NC2=O)c2cc(F)c(N3CC4(CC(N5CCC6(CC5)CC(c5cnc7ccc(Oc8c(F)ccc(NS(=O)(=O)N9CC[C@@H](F)C9)c8C#N)cc7n5)C6)C4)C3)cc21]

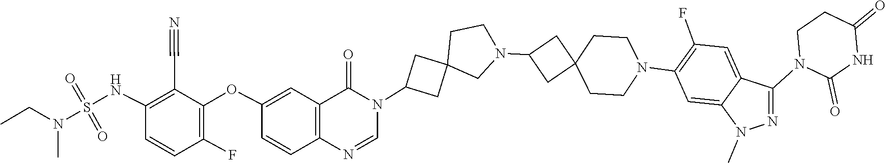 [SMILES: CCN(C)S(=O)(=O)Nc1ccc(F)c(Oc2ccc3ncn(C4CC5(CCN(C6CC7(CCN(c8cc9c(cc8F)c(N8CCC(=O)NC8=O)nn9C)CC7)C6)C5)C4)c(=O)c3c2)c1C#N]

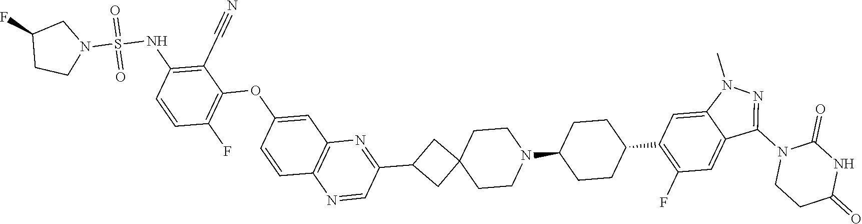 [SMILES: Cn1nc(N2CCC(=O)NC2=O)c2cc(F)c([C@H]3CC[C@H](N4CCC5(CC4)CC(c4cnc6ccc(Oc7c(F)ccc(NS(=O)(=O)N8CC[C@@H](F)C8)c7C#N)cc6n4)C5)CC3)cc21]

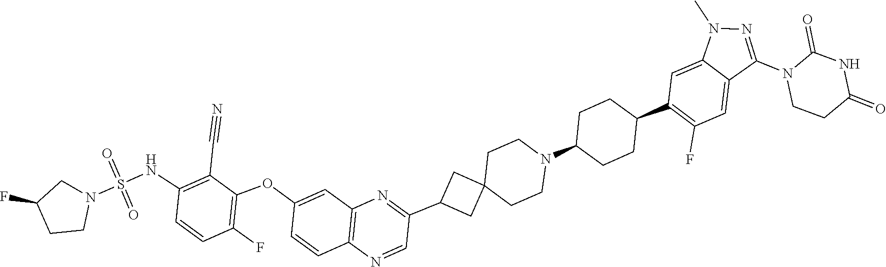 [SMILES: Cn1nc(N2CCC(=O)NC2=O)c2cc(F)c([C@H]3CC[C@@H](N4CCC5(CC4)CC(c4cnc6ccc(Oc7c(F)ccc(NS(=O)(=O)N8CC[C@@H](F)C8)c7C#N)cc6n4)C5)CC3)cc21]

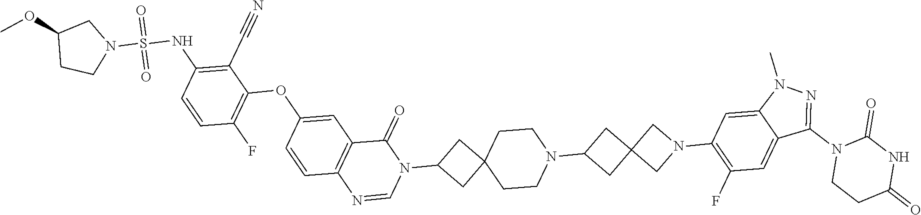 [SMILES: CO[C@@H]1CCN(S(=O)(=O)Nc2ccc(F)c(Oc3ccc4ncn(C5CC6(CCN(C7CC8(C7)CN(c7cc9c(cc7F)c(N7CCC(=O)NC7=O)nn9C)C8)CC6)C5)c(=O)c4c3)c2C#N)C1]

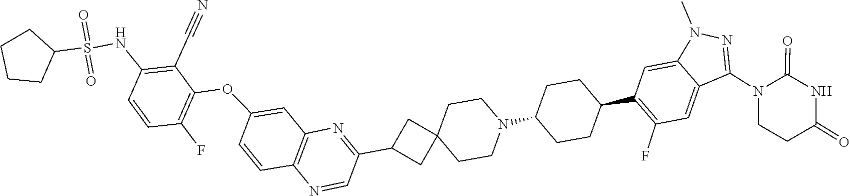 [SMILES: Cn1nc(N2CCC(=O)NC2=O)c2cc(F)c([C@H]3CC[C@H](N4CCC5(CC4)CC(c4cnc6ccc(Oc7c(F)ccc(NS(=O)(=O)C8CCCC8)c7C#N)cc6n4)C5)CC3)cc21]

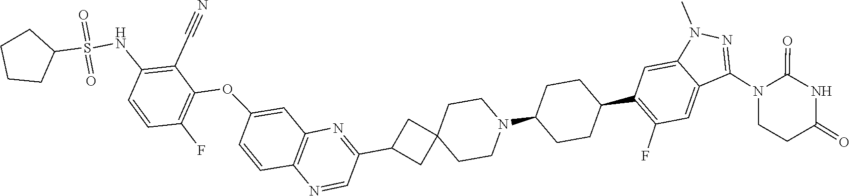 [SMILES: Cn1nc(N2CCC(=O)NC2=O)c2cc(F)c([C@H]3CC[C@@H](N4CCC5(CC4)CC(c4cnc6ccc(Oc7c(F)ccc(NS(=O)(=O)C8CCCC8)c7C#N)cc6n4)C5)CC3)cc21]

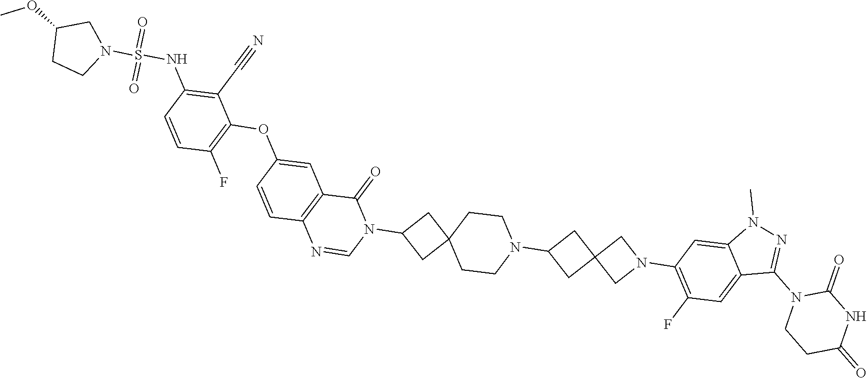 [SMILES: CO[C@H]1CCN(S(=O)(=O)Nc2ccc(F)c(Oc3ccc4ncn(C5CC6(CCN(C7CC8(C7)CN(c7cc9c(cc7F)c(N7CCC(=O)NC7=O)nn9C)C8)CC6)C5)c(=O)c4c3)c2C#N)C1]

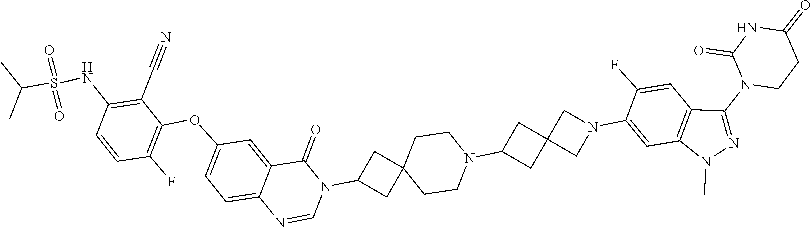 [SMILES: CC(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1C#N]

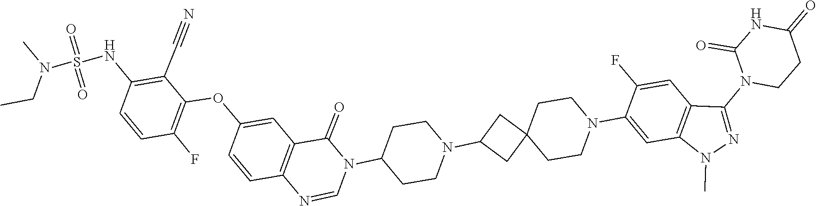 [SMILES: CCN(C)S(=O)(=O)Nc1ccc(F)c(Oc2ccc3ncn(C4CCN(C5CC6(CCN(c7cc8c(cc7F)c(N7CCC(=O)NC7=O)nn8C)CC6)C5)CC4)c(=O)c3c2)c1C#N]

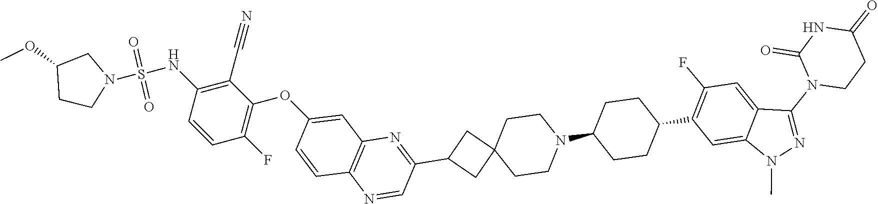 [SMILES: CO[C@H]1CCN(S(=O)(=O)Nc2ccc(F)c(Oc3ccc4ncc(C5CC6(CCN([C@H]7CC[C@H](c8cc9c(cc8F)c(N8CCC(=O)NC8=O)nn9C)CC7)CC6)C5)nc4c3)c2C#N)C1]

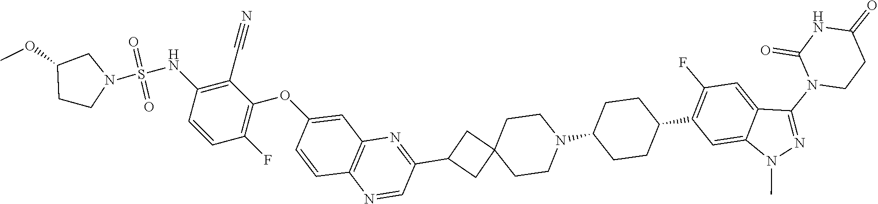 [SMILES: CO[C@H]1CCN(S(=O)(=O)Nc2ccc(F)c(Oc3ccc4ncc(C5CC6(CCN([C@H]7CC[C@@H](c8cc9c(cc8F)c(N8CCC(=O)NC8=O)nn9C)CC7)CC6)C5)nc4c3)c2C#N)C1]

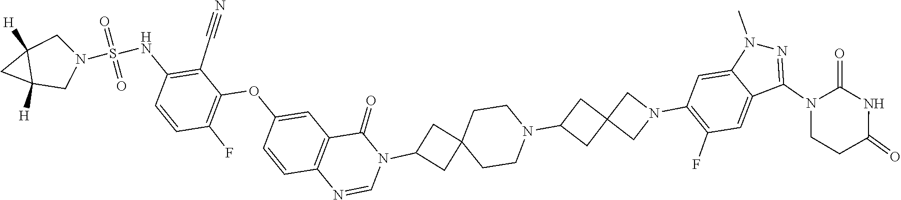 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9C[C@H]%10C[C@H]%10C9)c8C#N)cc7c5=O)C6)C4)C3)cc21]

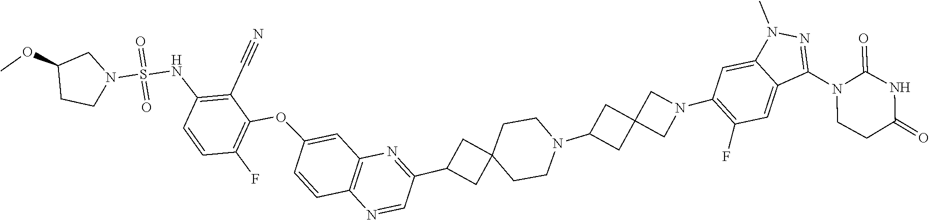 [SMILES: CO[C@@H]1CCN(S(=O)(=O)Nc2ccc(F)c(Oc3ccc4ncc(C5CC6(CCN(C7CC8(C7)CN(c7cc9c(cc7F)c(N7CCC(=O)NC7=O)nn9C)C8)CC6)C5)nc4c3)c2C#N)C1]

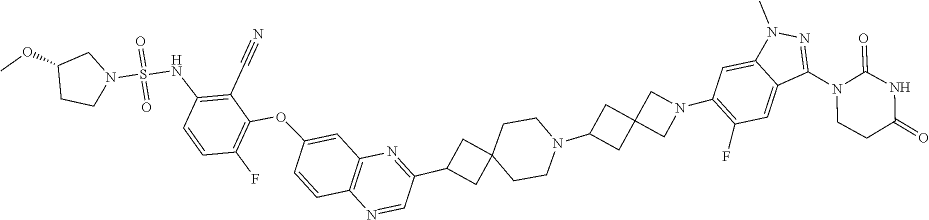 [SMILES: CO[C@H]1CCN(S(=O)(=O)Nc2ccc(F)c(Oc3ccc4ncc(C5CC6(CCN(C7CC8(C7)CN(c7cc9c(cc7F)c(N7CCC(=O)NC7=O)nn9C)C8)CC6)C5)nc4c3)c2C#N)C1]

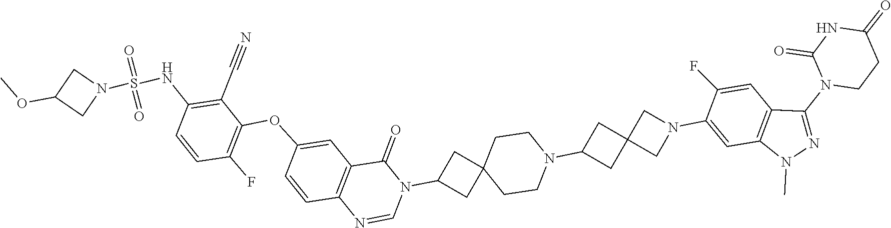 [SMILES: COC1CN(S(=O)(=O)Nc2ccc(F)c(Oc3ccc4ncn(C5CC6(CCN(C7CC8(C7)CN(c7cc9c(cc7F)c(N7CCC(=O)NC7=O)nn9C)C8)CC6)C5)c(=O)c4c3)c2C#N)C1]

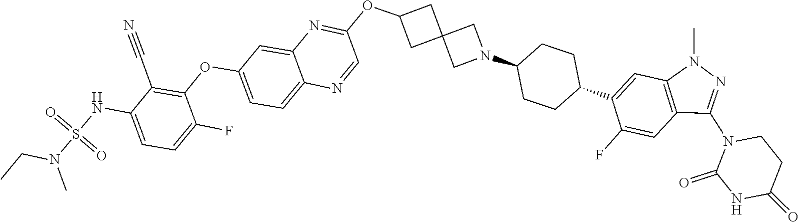 [SMILES: CCN(C)S(=O)(=O)Nc1ccc(F)c(Oc2ccc3ncc(OC4CC5(C4)CN([C@H]4CC[C@H](c6cc7c(cc6F)c(N6CCC(=O)NC6=O)nn7C)CC4)C5)nc3c2)c1C#N]

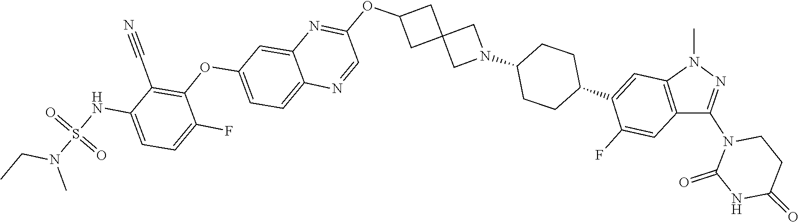 [SMILES: CCN(C)S(=O)(=O)Nc1ccc(F)c(Oc2ccc3ncc(OC4CC5(C4)CN([C@H]4CC[C@@H](c6cc7c(cc6F)c(N6CCC(=O)NC6=O)nn7C)CC4)C5)nc3c2)c1C#N]

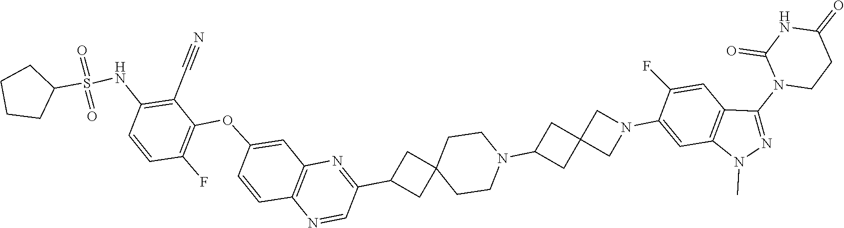 [SMILES: Cn1nc(N2CCC(=O)NC2=O)c2cc(F)c(N3CC4(CC(N5CCC6(CC5)CC(c5cnc7ccc(Oc8c(F)ccc(NS(=O)(=O)C9CCCC9)c8C#N)cc7n5)C6)C4)C3)cc21]

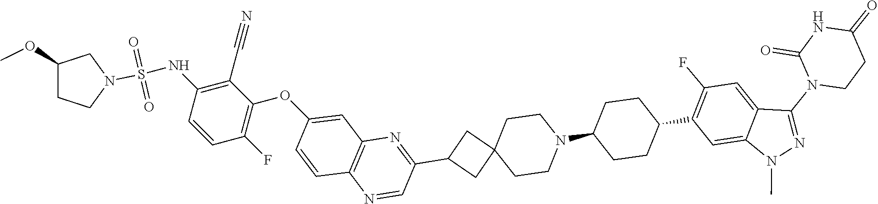 [SMILES: CO[C@@H]1CCN(S(=O)(=O)Nc2ccc(F)c(Oc3ccc4ncc(C5CC6(CCN([C@H]7CC[C@H](c8cc9c(cc8F)c(N8CCC(=O)NC8=O)nn9C)CC7)CC6)C5)nc4c3)c2C#N)C1]

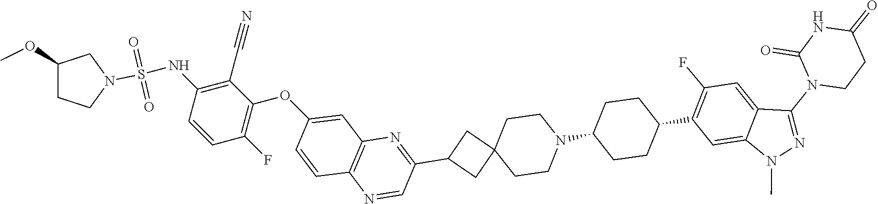 [SMILES: CO[C@@H]1CCN(S(=O)(=O)Nc2ccc(F)c(Oc3ccc4ncc(C5CC6(CCN([C@H]7CC[C@@H](c8cc9c(cc8F)c(N8CCC(=O)NC8=O)nn9C)CC7)CC6)C5)nc4c3)c2C#N)C1]

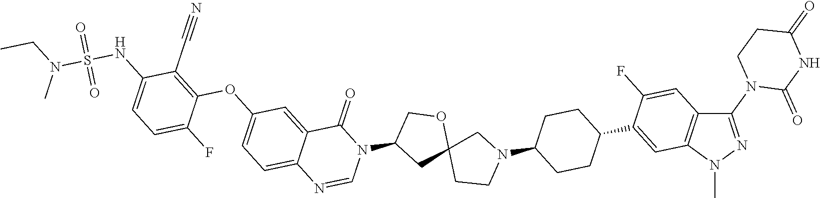 [SMILES: CCN(C)S(=O)(=O)Nc1ccc(F)c(Oc2ccc3ncn([C@H]4CO[C@]5(CCN([C@H]6CC[C@H](c7cc8c(cc7F)c(N7CCC(=O)NC7=O)nn8C)CC6)C5)C4)c(=O)c3c2)c1C#N]

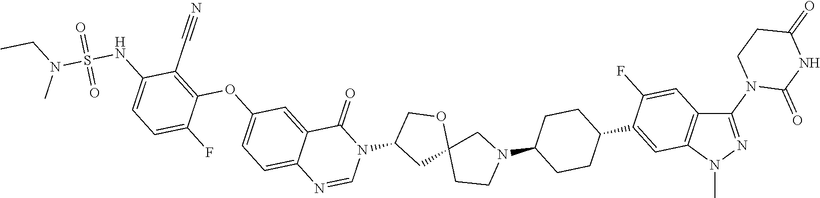 [SMILES: CCN(C)S(=O)(=O)Nc1ccc(F)c(Oc2ccc3ncn([C@@H]4CO[C@@]5(CCN([C@H]6CC[C@H](c7cc8c(cc7F)c(N7CCC(=O)NC7=O)nn8C)CC6)C5)C4)c(=O)c3c2)c1C#N]

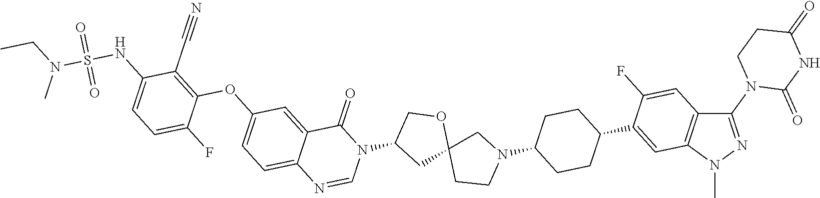 [SMILES: CCN(C)S(=O)(=O)Nc1ccc(F)c(Oc2ccc3ncn([C@@H]4CO[C@@]5(CCN([C@H]6CC[C@@H](c7cc8c(cc7F)c(N7CCC(=O)NC7=O)nn8C)CC6)C5)C4)c(=O)c3c2)c1C#N]

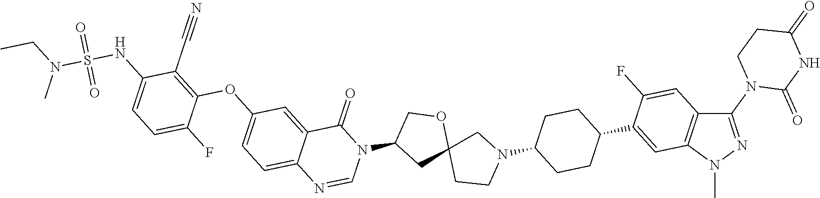 [SMILES: CCN(C)S(=O)(=O)Nc1ccc(F)c(Oc2ccc3ncn([C@H]4CO[C@]5(CCN([C@H]6CC[C@@H](c7cc8c(cc7F)c(N7CCC(=O)NC7=O)nn8C)CC6)C5)C4)c(=O)c3c2)c1C#N]

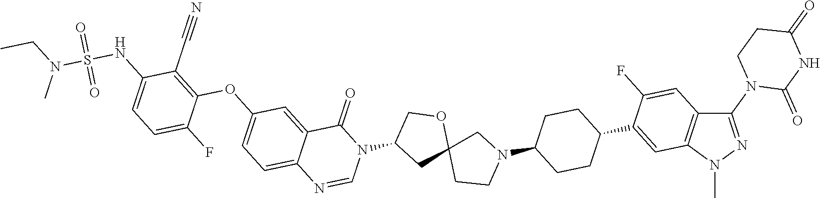 [SMILES: CCN(C)S(=O)(=O)Nc1ccc(F)c(Oc2ccc3ncn([C@@H]4CO[C@]5(CCN([C@H]6CC[C@H](c7cc8c(cc7F)c(N7CCC(=O)NC7=O)nn8C)CC6)C5)C4)c(=O)c3c2)c1C#N]

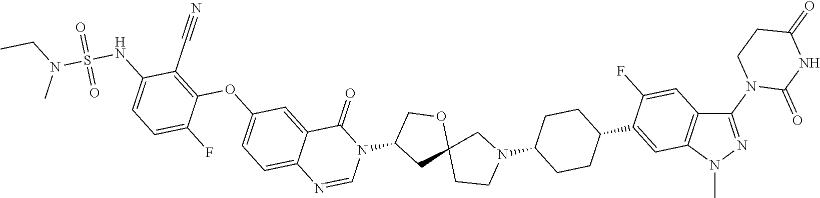 [SMILES: CCN(C)S(=O)(=O)Nc1ccc(F)c(Oc2ccc3ncn([C@@H]4CO[C@]5(CCN([C@H]6CC[C@@H](c7cc8c(cc7F)c(N7CCC(=O)NC7=O)nn8C)CC6)C5)C4)c(=O)c3c2)c1C#N]

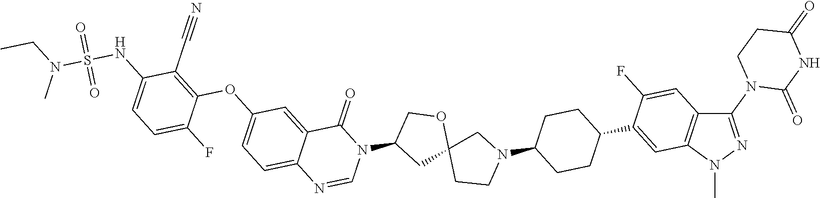 [SMILES: CCN(C)S(=O)(=O)Nc1ccc(F)c(Oc2ccc3ncn([C@H]4CO[C@@]5(CCN([C@H]6CC[C@H](c7cc8c(cc7F)c(N7CCC(=O)NC7=O)nn8C)CC6)C5)C4)c(=O)c3c2)c1C#N]

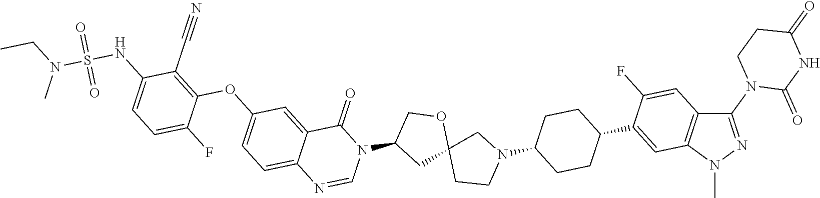 [SMILES: CCN(C)S(=O)(=O)Nc1ccc(F)c(Oc2ccc3ncn([C@H]4CO[C@@]5(CCN([C@H]6CC[C@@H](c7cc8c(cc7F)c(N7CCC(=O)NC7=O)nn8C)CC6)C5)C4)c(=O)c3c2)c1C#N]

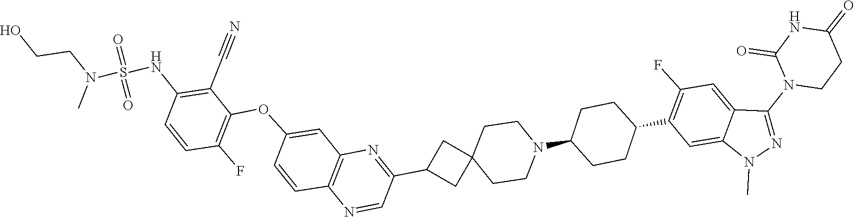 [SMILES: CN(CCO)S(=O)(=O)Nc1ccc(F)c(Oc2ccc3ncc(C4CC5(CCN([C@H]6CC[C@H](c7cc8c(cc7F)c(N7CCC(=O)NC7=O)nn8C)CC6)CC5)C4)nc3c2)c1C#N]

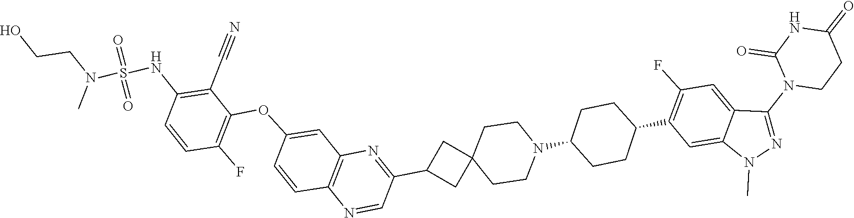 [SMILES: CN(CCO)S(=O)(=O)Nc1ccc(F)c(Oc2ccc3ncc(C4CC5(CCN([C@H]6CC[C@@H](c7cc8c(cc7F)c(N7CCC(=O)NC7=O)nn8C)CC6)CC5)C4)nc3c2)c1C#N]

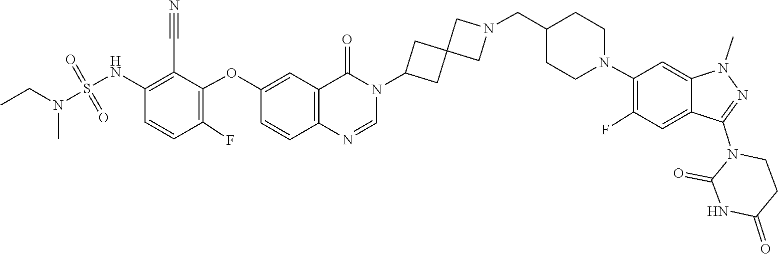 [SMILES: CCN(C)S(=O)(=O)Nc1ccc(F)c(Oc2ccc3ncn(C4CC5(C4)CN(CC4CCN(c6cc7c(cc6F)c(N6CCC(=O)NC6=O)nn7C)CC4)C5)c(=O)c3c2)c1C#N]

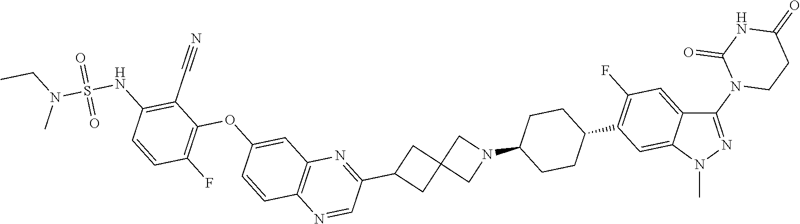 [SMILES: CCN(C)S(=O)(=O)Nc1ccc(F)c(Oc2ccc3ncc(C4CC5(C4)CN([C@H]4CC[C@H](c6cc7c(cc6F)c(N6CCC(=O)NC6=O)nn7C)CC4)C5)nc3c2)c1C#N]

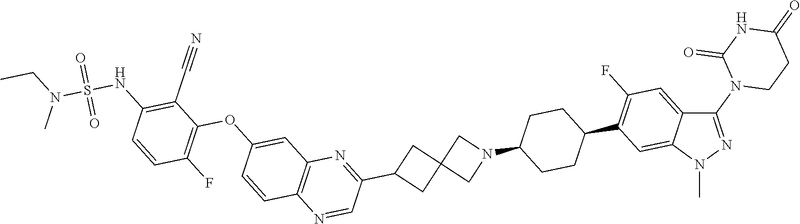 [SMILES: CCN(C)S(=O)(=O)Nc1ccc(F)c(Oc2ccc3ncc(C4CC5(C4)CN([C@H]4CC[C@@H](c6cc7c(cc6F)c(N6CCC(=O)NC6=O)nn7C)CC4)C5)nc3c2)c1C#N]

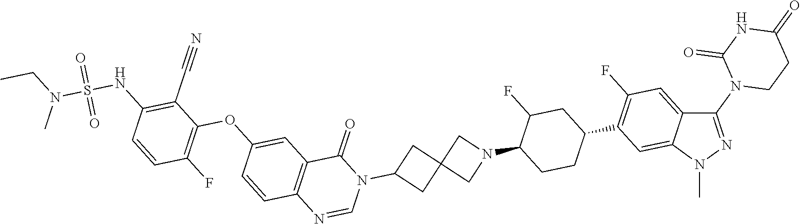 [SMILES: CCN(C)S(=O)(=O)Nc1ccc(F)c(Oc2ccc3ncn(C4CC5(C4)CN([C@@H]4CC[C@@H](c6cc7c(cc6F)c(N6CCC(=O)NC6=O)nn7C)CC4F)C5)c(=O)c3c2)c1C#N]

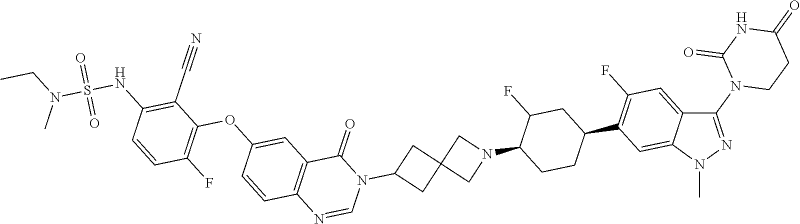 [SMILES: CCN(C)S(=O)(=O)Nc1ccc(F)c(Oc2ccc3ncn(C4CC5(C4)CN([C@@H]4CC[C@H](c6cc7c(cc6F)c(N6CCC(=O)NC6=O)nn7C)CC4F)C5)c(=O)c3c2)c1C#N]

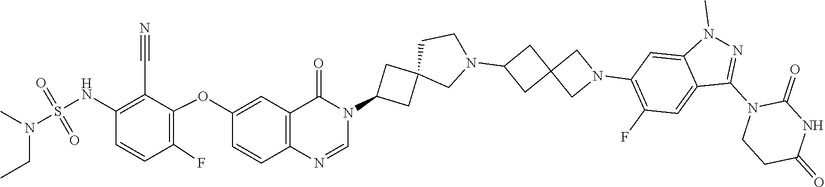 [SMILES: CCN(C)S(=O)(=O)Nc1ccc(F)c(Oc2ccc3ncn([C@H]4C[C@]5(CCN(C6CC7(C6)CN(c6cc8c(cc6F)c(N6CCC(=O)NC6=O)nn8C)C7)C5)C4)c(=O)c3c2)c1C#N]

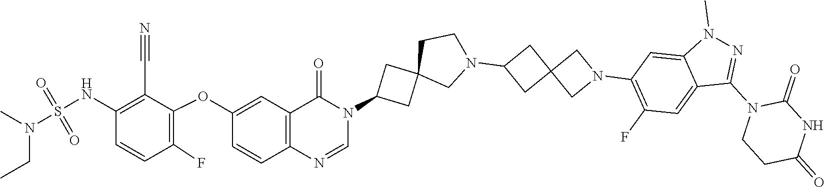 [SMILES: CCN(C)S(=O)(=O)Nc1ccc(F)c(Oc2ccc3ncn([C@H]4C[C@@]5(CCN(C6CC7(C6)CN(c6cc8c(cc6F)c(N6CCC(=O)NC6=O)nn8C)C7)C5)C4)c(=O)c3c2)c1C#N]

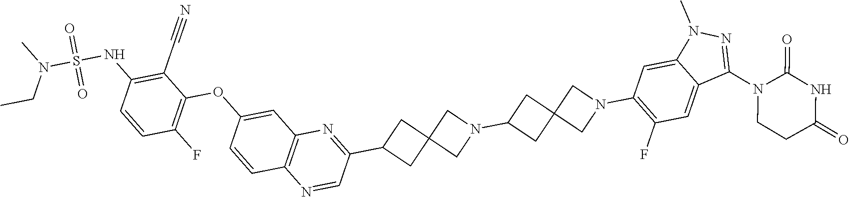 [SMILES: CCN(C)S(=O)(=O)Nc1ccc(F)c(Oc2ccc3ncc(C4CC5(C4)CN(C4CC6(C4)CN(c4cc7c(cc4F)c(N4CCC(=O)NC4=O)nn7C)C6)C5)nc3c2)c1C#N]

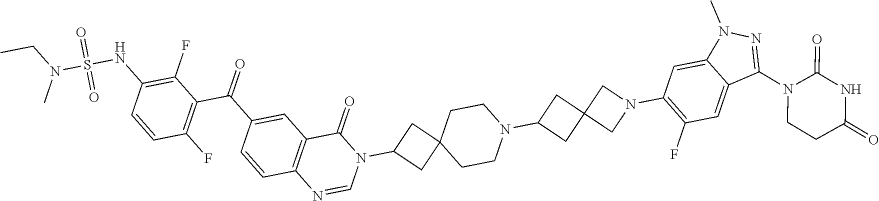 [SMILES: CCN(C)S(=O)(=O)Nc1ccc(F)c(C(=O)c2ccc3ncn(C4CC5(CCN(C6CC7(C6)CN(c6cc8c(cc6F)c(N6CCC(=O)NC6=O)nn8C)C7)CC5)C4)c(=O)c3c2)c1F]

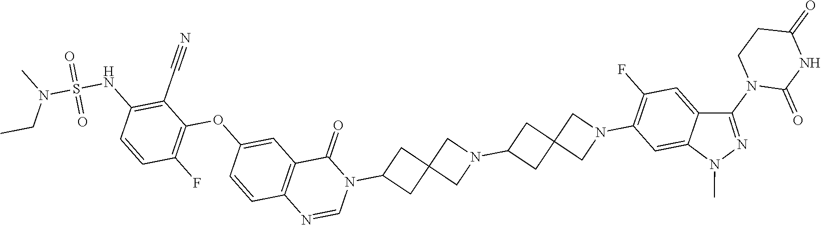 [SMILES: CCN(C)S(=O)(=O)Nc1ccc(F)c(Oc2ccc3ncn(C4CC5(C4)CN(C4CC6(C4)CN(c4cc7c(cc4F)c(N4CCC(=O)NC4=O)nn7C)C6)C5)c(=O)c3c2)c1C#N]

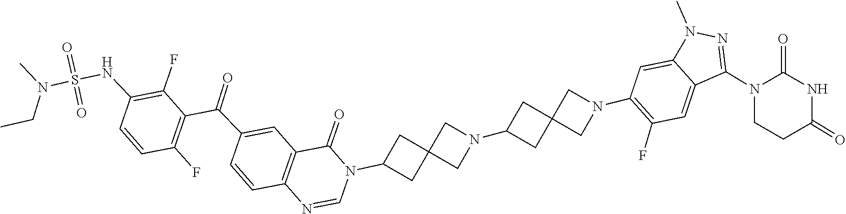 [SMILES: CCN(C)S(=O)(=O)Nc1ccc(F)c(C(=O)c2ccc3ncn(C4CC5(C4)CN(C4CC6(C4)CN(c4cc7c(cc4F)c(N4CCC(=O)NC4=O)nn7C)C6)C5)c(=O)c3c2)c1F]

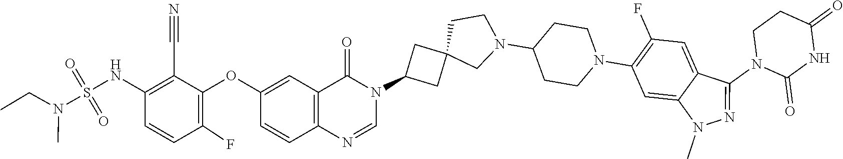 [SMILES: CCN(C)S(=O)(=O)Nc1ccc(F)c(Oc2ccc3ncn([C@H]4C[C@]5(CCN(C6CCN(c7cc8c(cc7F)c(N7CCC(=O)NC7=O)nn8C)CC6)C5)C4)c(=O)c3c2)c1C#N]

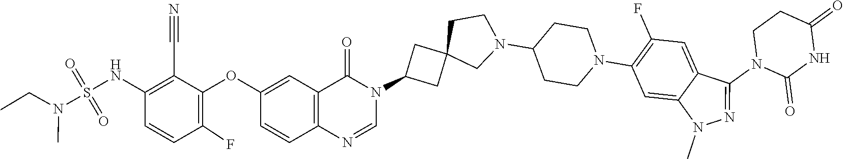 [SMILES: CCN(C)S(=O)(=O)Nc1ccc(F)c(Oc2ccc3ncn([C@H]4C[C@@]5(CCN(C6CCN(c7cc8c(cc7F)c(N7CCC(=O)NC7=O)nn8C)CC6)C5)C4)c(=O)c3c2)c1C#N]

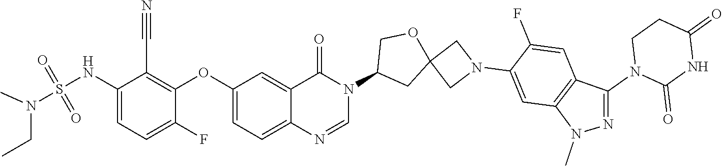 [SMILES: CCN(C)S(=O)(=O)Nc1ccc(F)c(Oc2ccc3ncn([C@H]4COC5(C4)CN(c4cc6c(cc4F)c(N4CCC(=O)NC4=O)nn6C)C5)c(=O)c3c2)c1C#N]

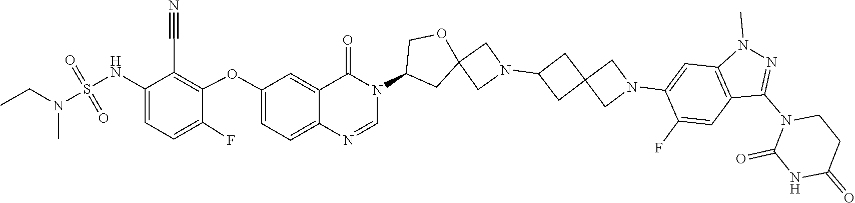 [SMILES: CCN(C)S(=O)(=O)Nc1ccc(F)c(Oc2ccc3ncn([C@H]4COC5(C4)CN(C4CC6(C4)CN(c4cc7c(cc4F)c(N4CCC(=O)NC4=O)nn7C)C6)C5)c(=O)c3c2)c1C#N]

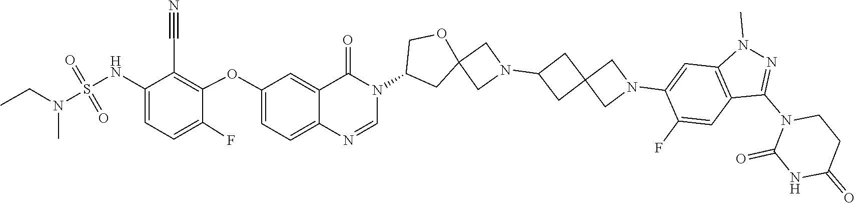 [SMILES: CCN(C)S(=O)(=O)Nc1ccc(F)c(Oc2ccc3ncn([C@@H]4COC5(C4)CN(C4CC6(C4)CN(c4cc7c(cc4F)c(N4CCC(=O)NC4=O)nn7C)C6)C5)c(=O)c3c2)c1C#N]

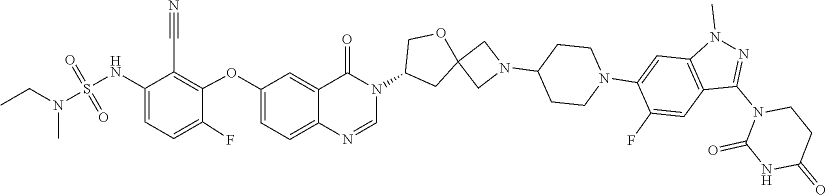 [SMILES: CCN(C)S(=O)(=O)Nc1ccc(F)c(Oc2ccc3ncn([C@@H]4COC5(C4)CN(C4CCN(c6cc7c(cc6F)c(N6CCC(=O)NC6=O)nn7C)CC4)C5)c(=O)c3c2)c1C#N]

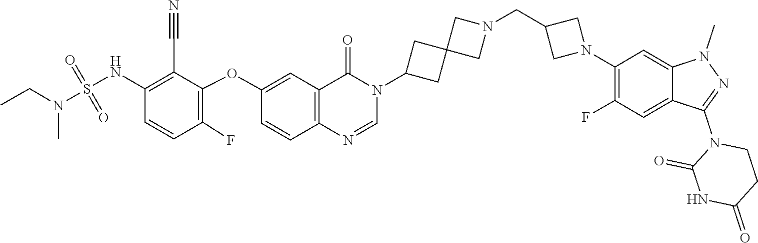 [SMILES: CCN(C)S(=O)(=O)Nc1ccc(F)c(Oc2ccc3ncn(C4CC5(C4)CN(CC4CN(c6cc7c(cc6F)c(N6CCC(=O)NC6=O)nn7C)C4)C5)c(=O)c3c2)c1C#N]

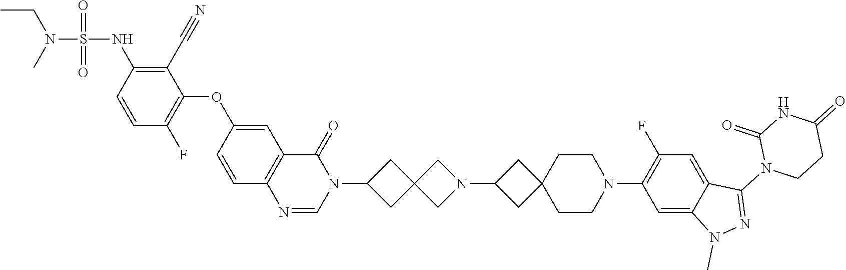 [SMILES: CCN(C)S(=O)(=O)Nc1ccc(F)c(Oc2ccc3ncn(C4CC5(C4)CN(C4CC6(CCN(c7cc8c(cc7F)c(N7CCC(=O)NC7=O)nn8C)CC6)C4)C5)c(=O)c3c2)c1C#N]

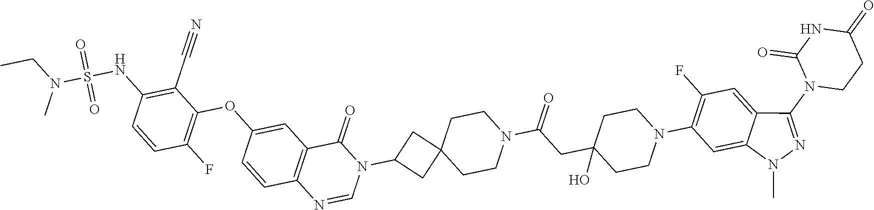 [SMILES: CCN(C)S(=O)(=O)Nc1ccc(F)c(Oc2ccc3ncn(C4CC5(CCN(C(=O)CC6(O)CCN(c7cc8c(cc7F)c(N7CCC(=O)NC7=O)nn8C)CC6)CC5)C4)c(=O)c3c2)c1C#N]

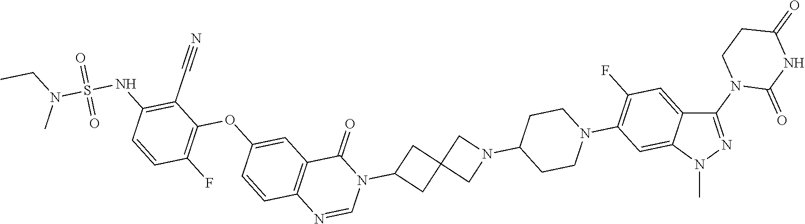 [SMILES: CCN(C)S(=O)(=O)Nc1ccc(F)c(Oc2ccc3ncn(C4CC5(C4)CN(C4CCN(c6cc7c(cc6F)c(N6CCC(=O)NC6=O)nn7C)CC4)C5)c(=O)c3c2)c1C#N]

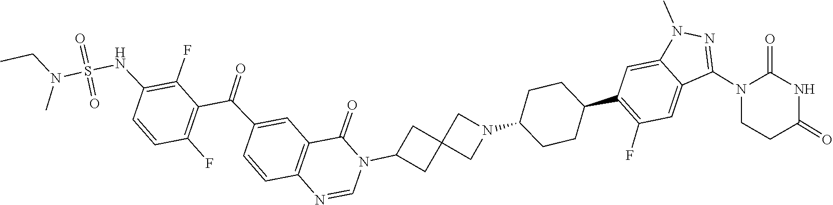 [SMILES: CCN(C)S(=O)(=O)Nc1ccc(F)c(C(=O)c2ccc3ncn(C4CC5(C4)CN([C@H]4CC[C@H](c6cc7c(cc6F)c(N6CCC(=O)NC6=O)nn7C)CC4)C5)c(=O)c3c2)c1F]

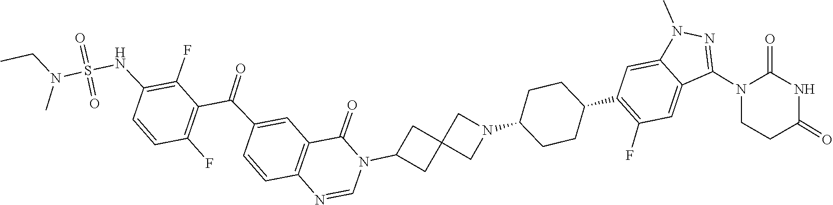 [SMILES: CCN(C)S(=O)(=O)Nc1ccc(F)c(C(=O)c2ccc3ncn(C4CC5(C4)CN([C@H]4CC[C@@H](c6cc7c(cc6F)c(N6CCC(=O)NC6=O)nn7C)CC4)C5)c(=O)c3c2)c1F]

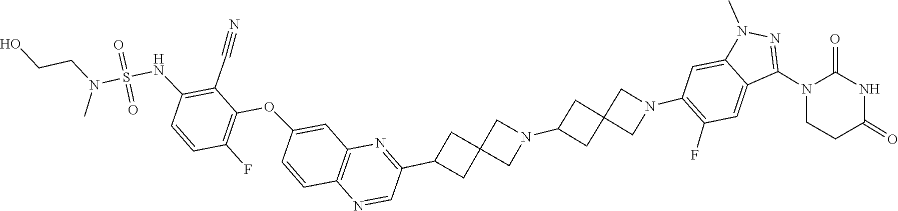 [SMILES: CN(CCO)S(=O)(=O)Nc1ccc(F)c(Oc2ccc3ncc(C4CC5(C4)CN(C4CC6(C4)CN(c4cc7c(cc4F)c(N4CCC(=O)NC4=O)nn7C)C6)C5)nc3c2)c1C#N]

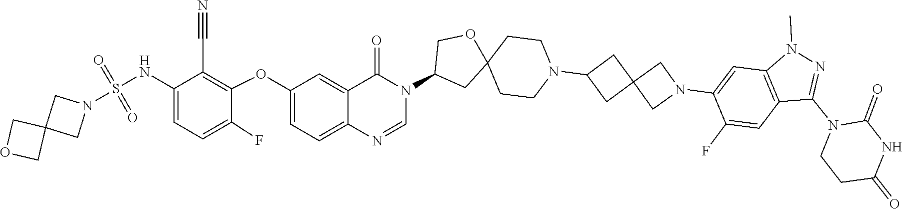 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC%10(COC%10)C9)c8C#N)cc7c5=O)CO6)C4)C3)cc21]

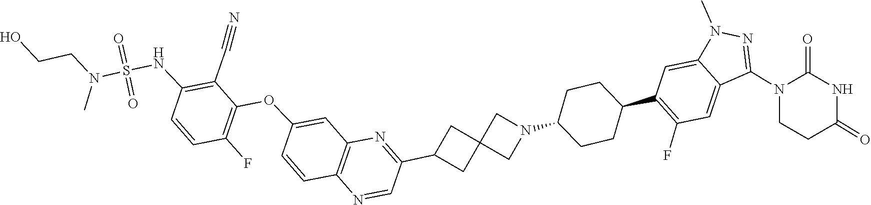 [SMILES: CN(CCO)S(=O)(=O)Nc1ccc(F)c(Oc2ccc3ncc(C4CC5(C4)CN([C@H]4CC[C@H](c6cc7c(cc6F)c(N6CCC(=O)NC6=O)nn7C)CC4)C5)nc3c2)c1C#N]

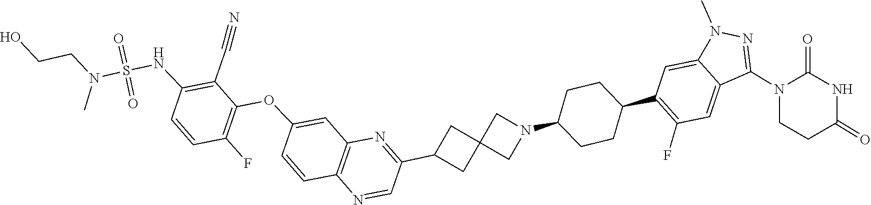 [SMILES: CN(CCO)S(=O)(=O)Nc1ccc(F)c(Oc2ccc3ncc(C4CC5(C4)CN([C@H]4CC[C@@H](c6cc7c(cc6F)c(N6CCC(=O)NC6=O)nn7C)CC4)C5)nc3c2)c1C#N]

Additional examples of compounds of the present invention include:

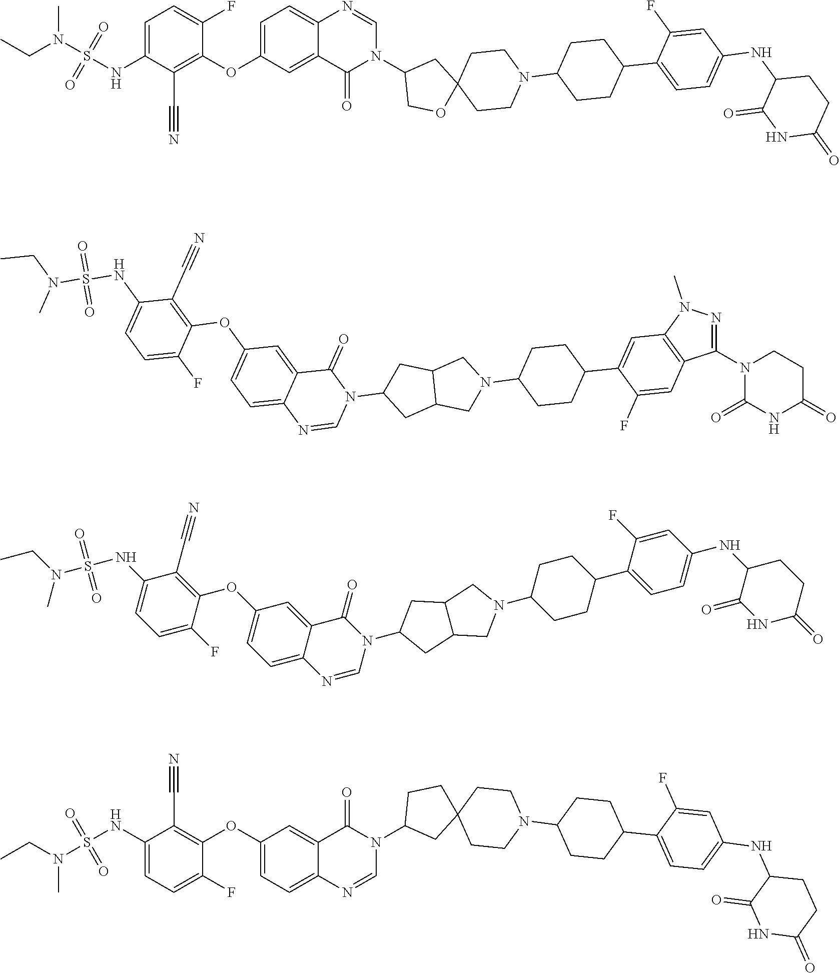 [SMILES: CCN(C)S(=O)(=O)Nc1ccc(F)c(Oc2ccc3ncn(C4COC5(CCN(C6CCC(c7ccc(NC8CCC(=O)NC8=O)cc7F)CC6)CC5)C4)c(=O)c3c2)c1C#N ; CCN(C)S(=O)(=O)Nc1ccc(F)c(Oc2ccc3ncn(C4CC5CN(C6CCC(c7cc8c(cc7F)c(N7CCC(=O)NC7=O)nn8C)CC6)CC5C4)c(=O)c3c2)c1C#N ; CCN(C)S(=O)(=O)Nc1ccc(F)c(Oc2ccc3ncn(C4CC5CN(C6CCC(c7ccc(NC8CCC(=O)NC8=O)cc7F)CC6)CC5C4)c(=O)c3c2)c1C#N ; CCN(C)S(=O)(=O)Nc1ccc(F)c(Oc2ccc3ncn(C4CCC5(CCN(C6CCC(c7ccc(NC8CCC(=O)NC8=O)cc7F)CC6)CC5)C4)c(=O)c3c2)c1C#N]

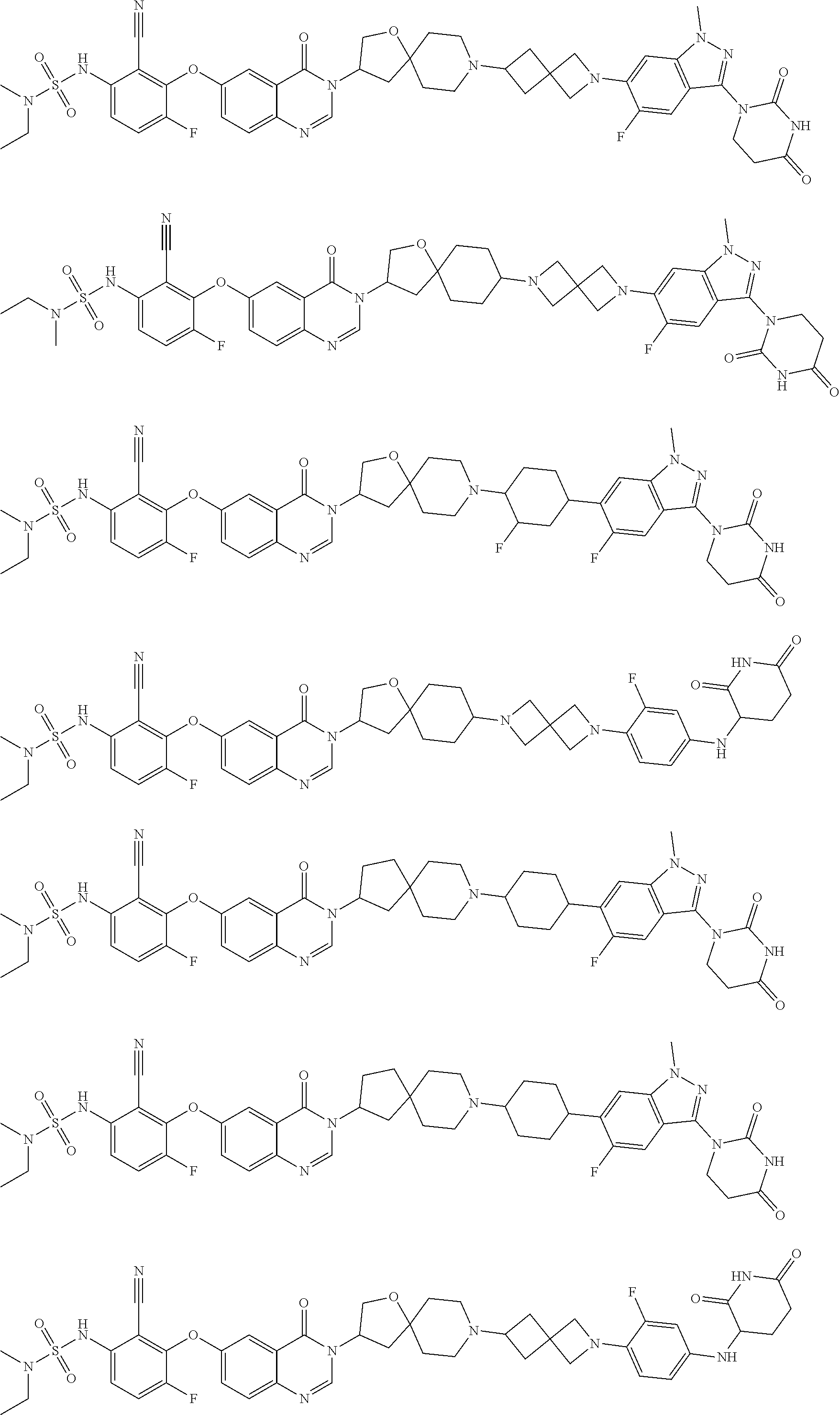

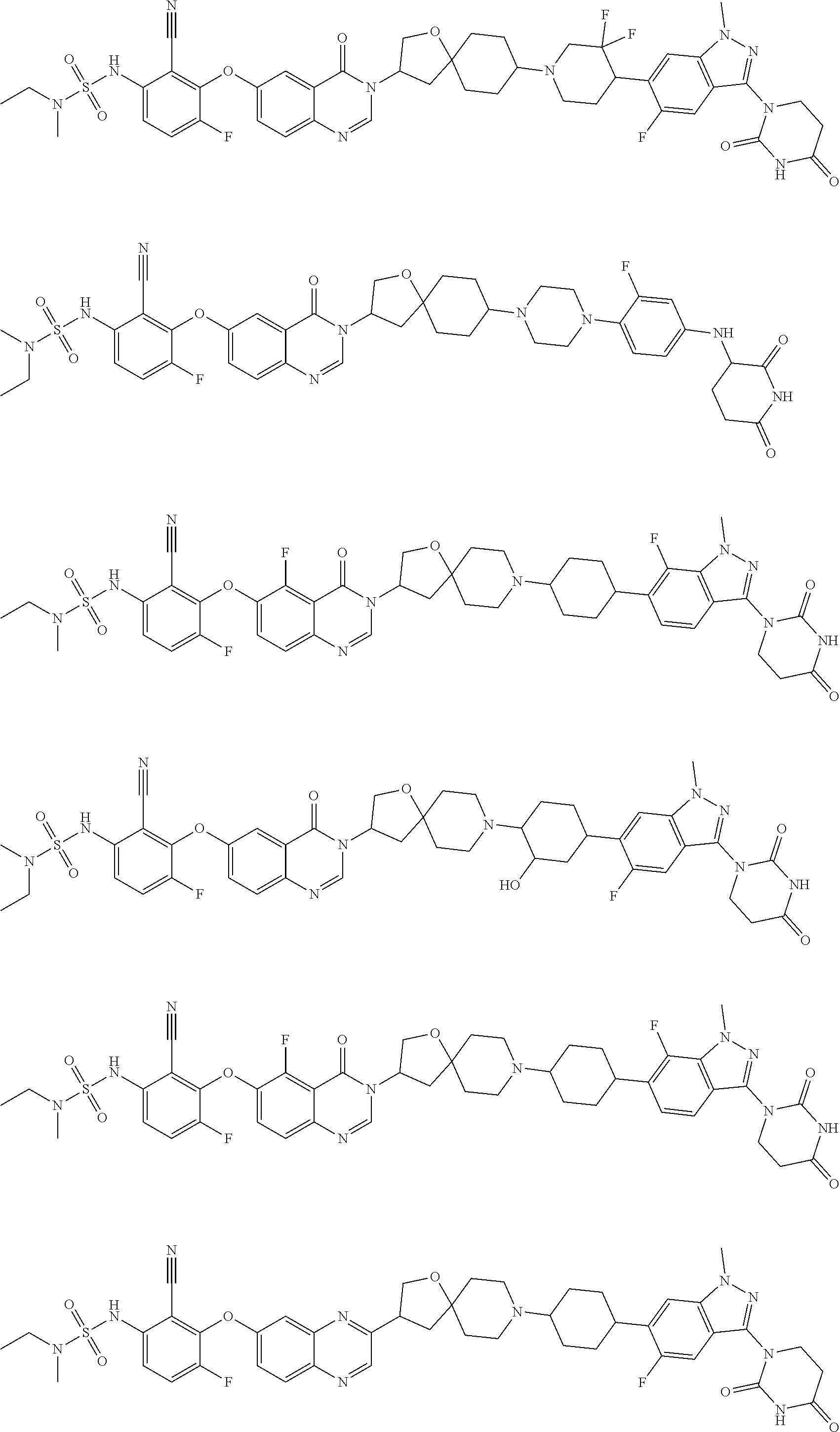

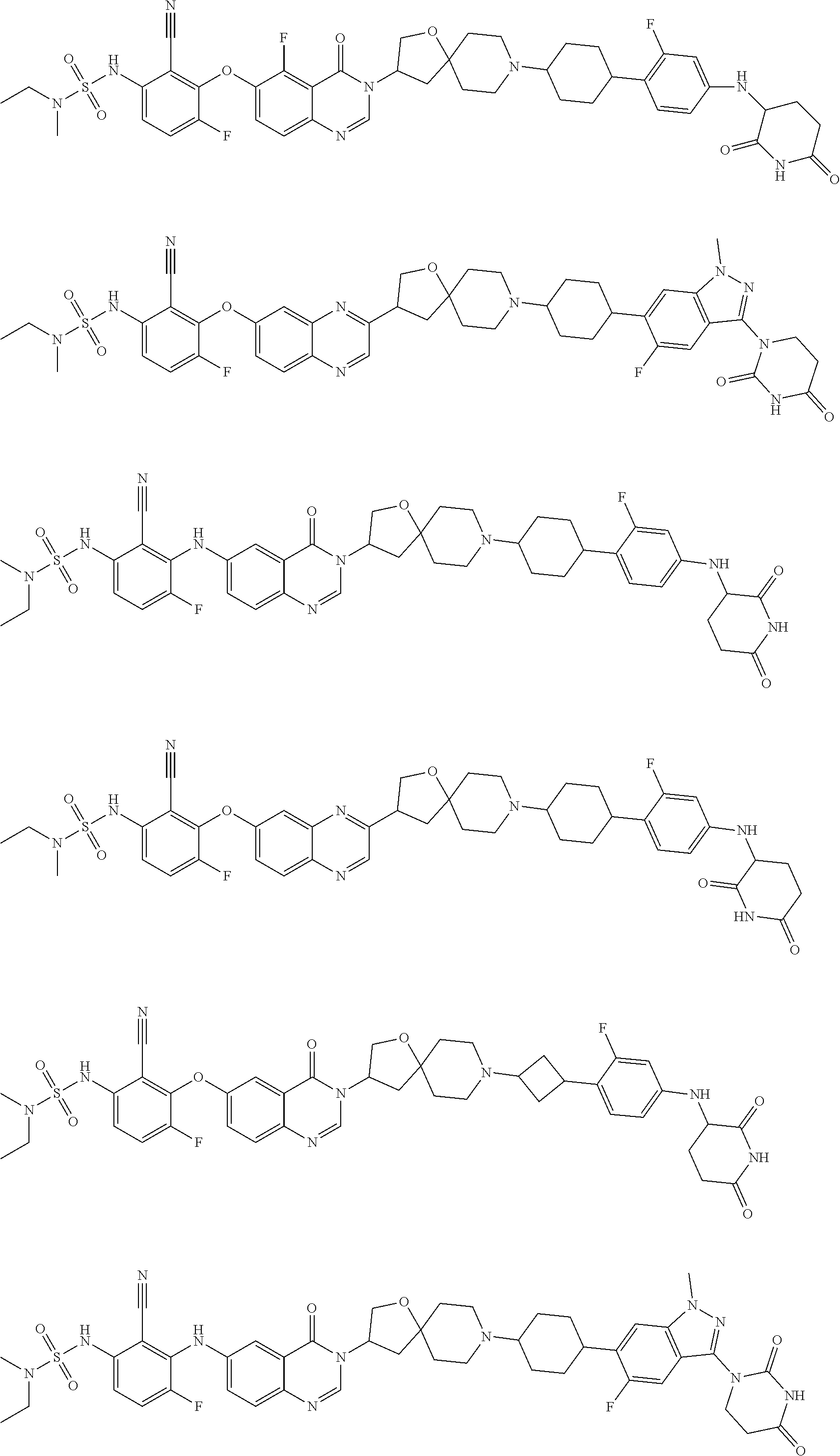

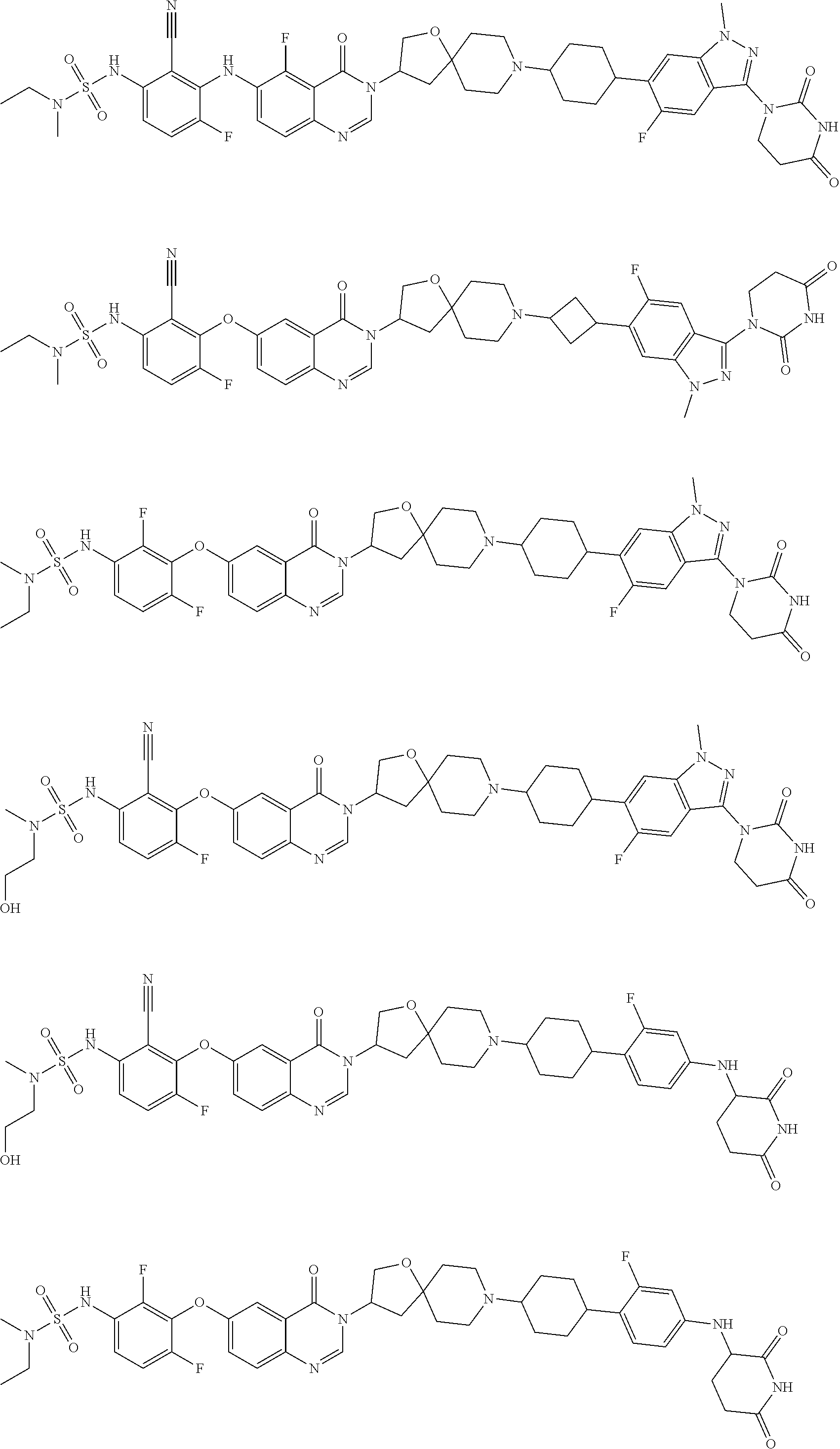

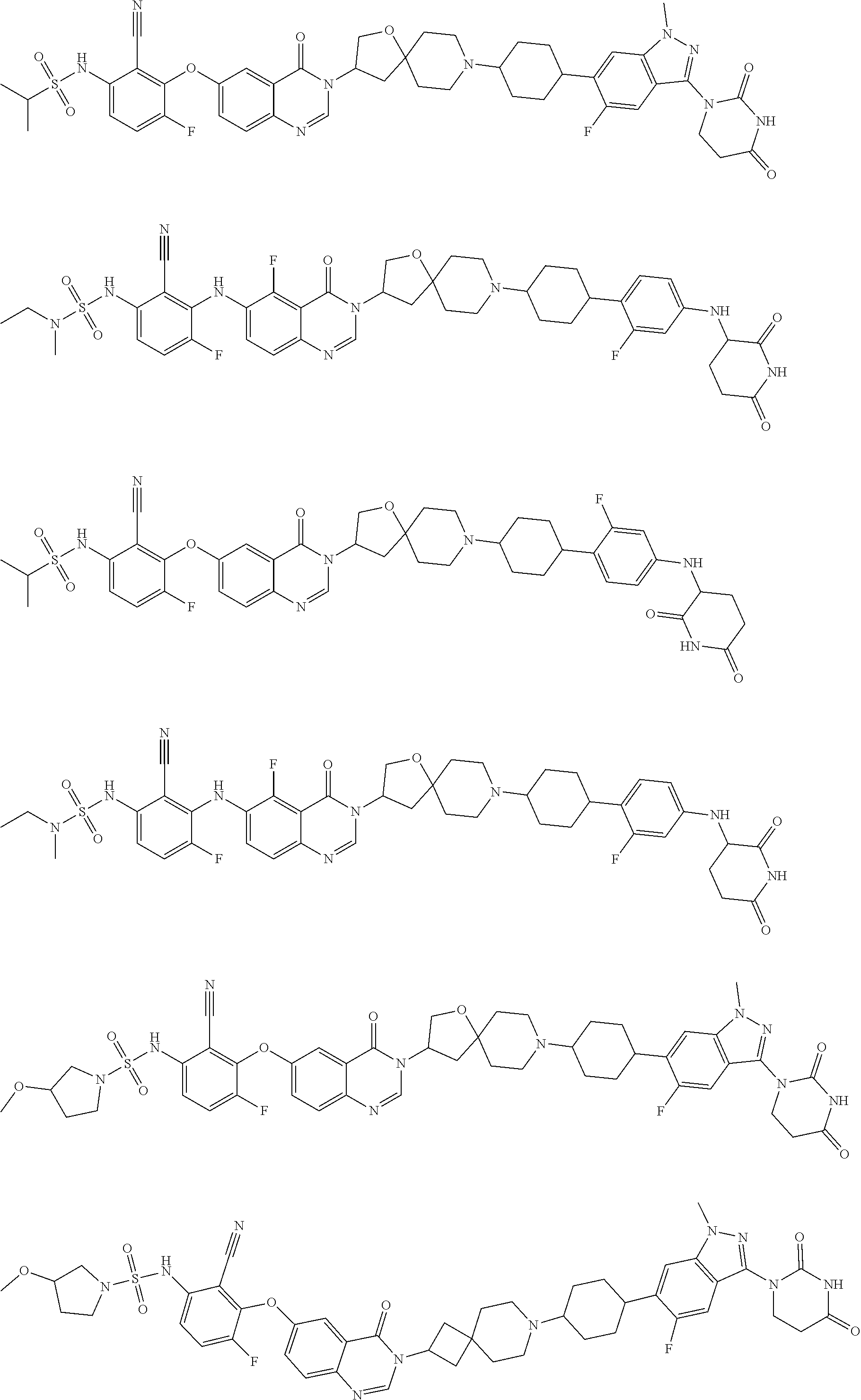

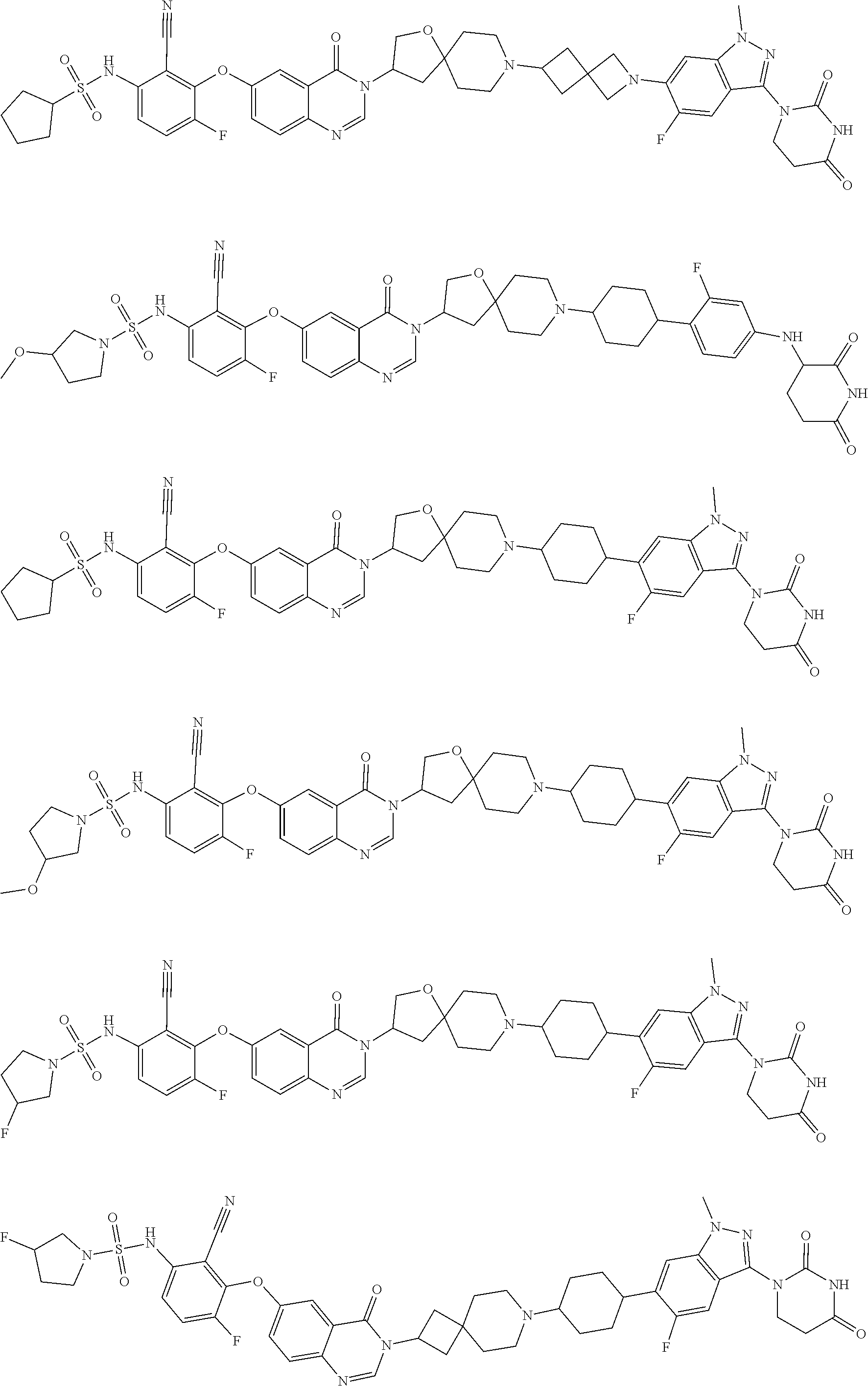

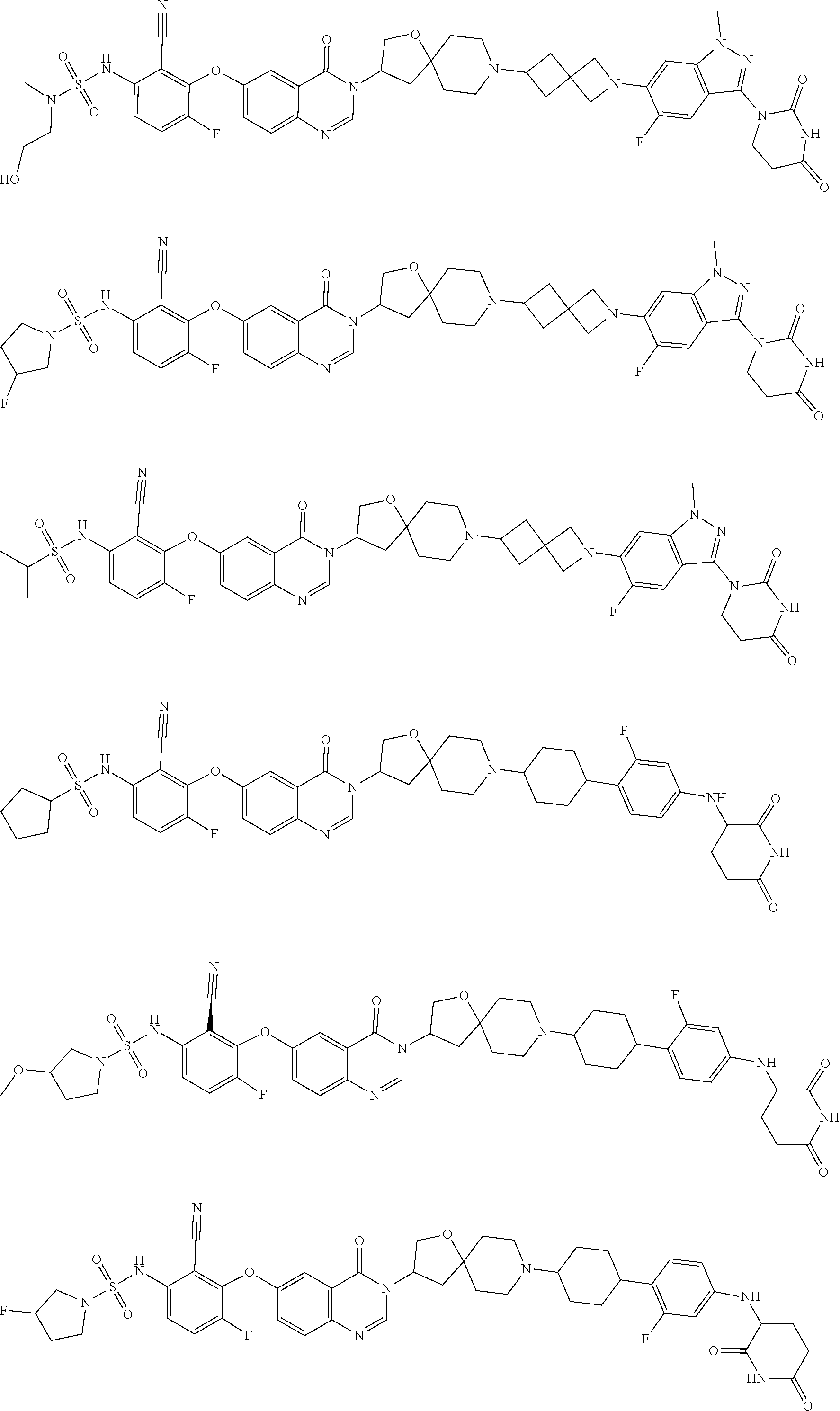

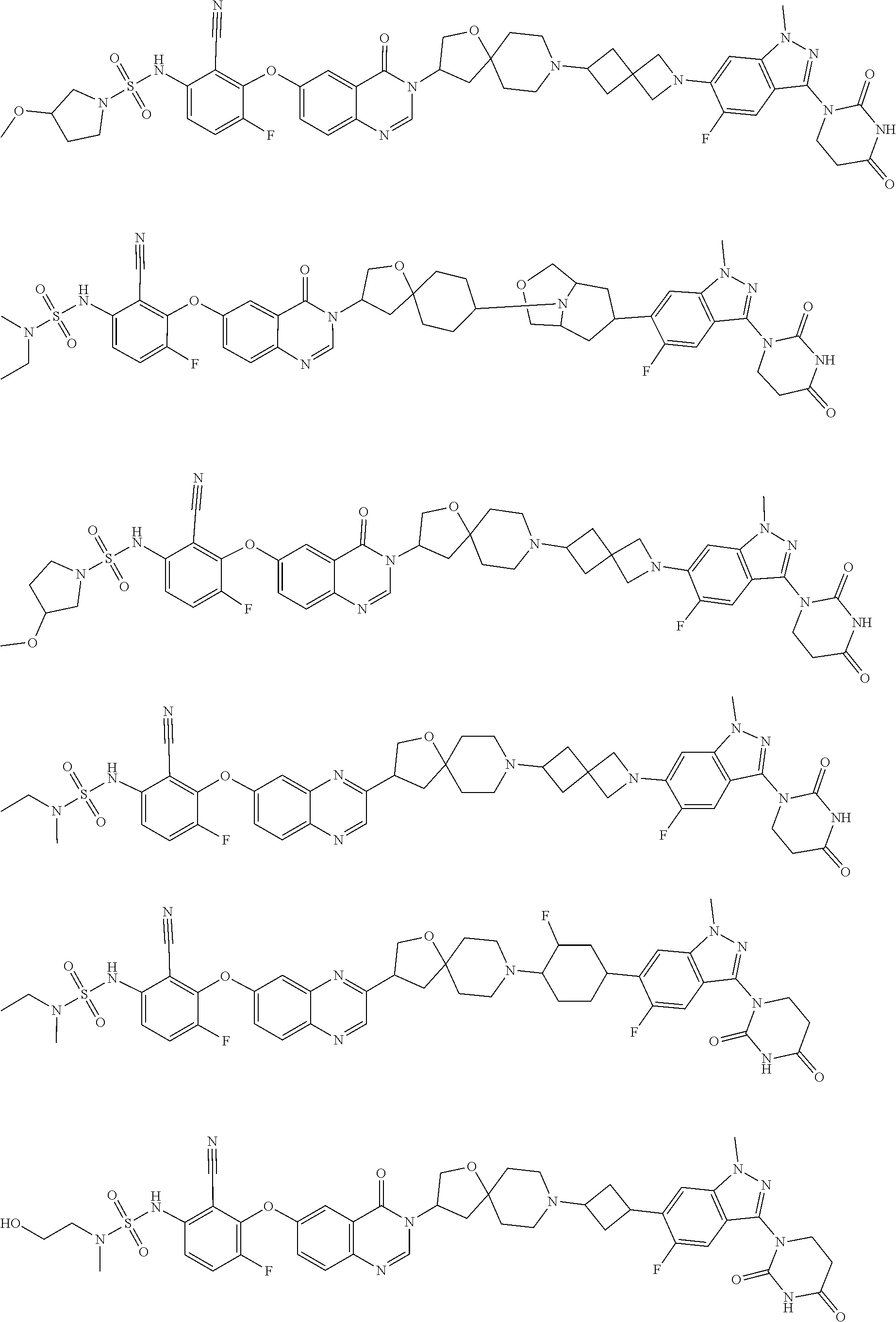

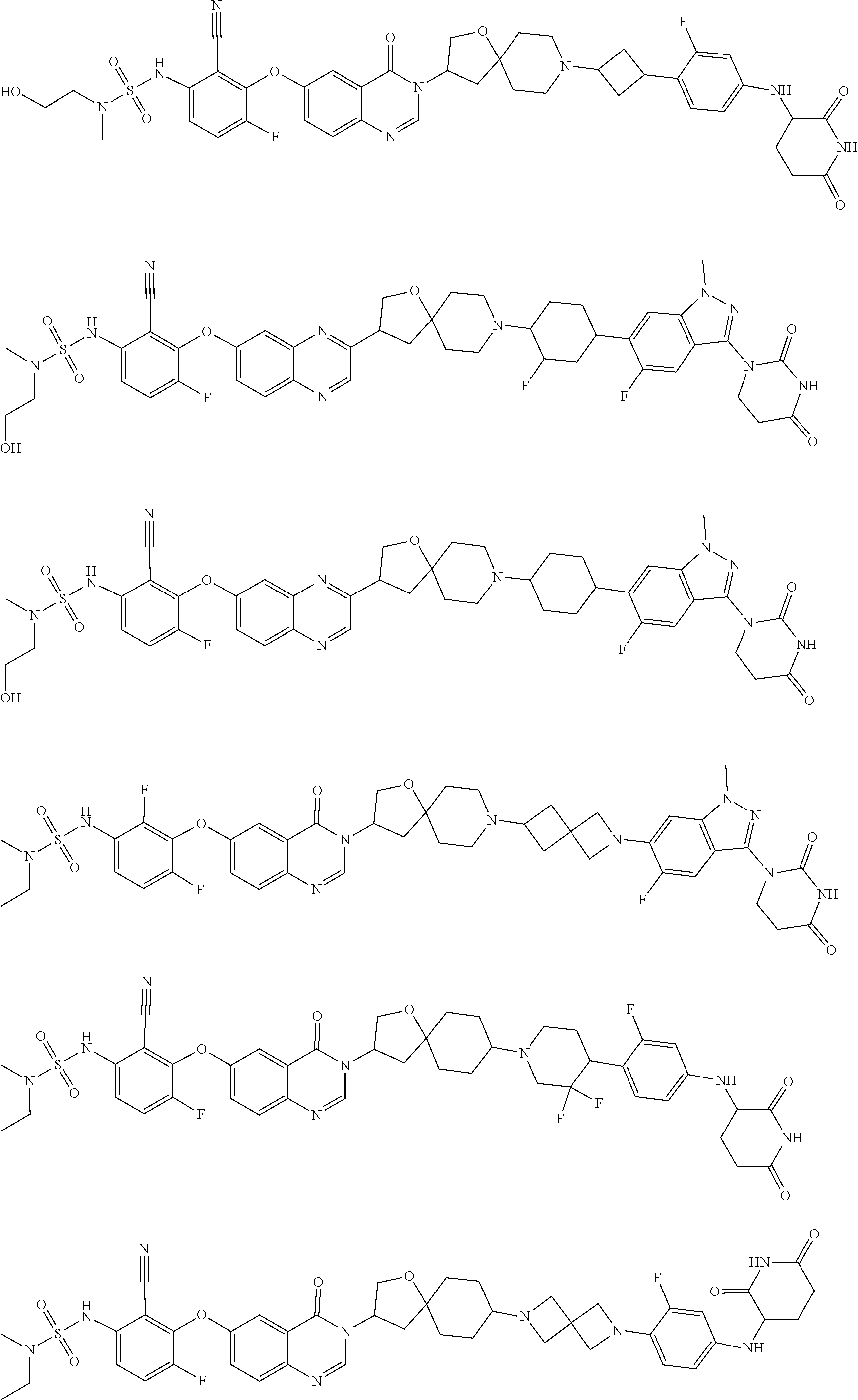

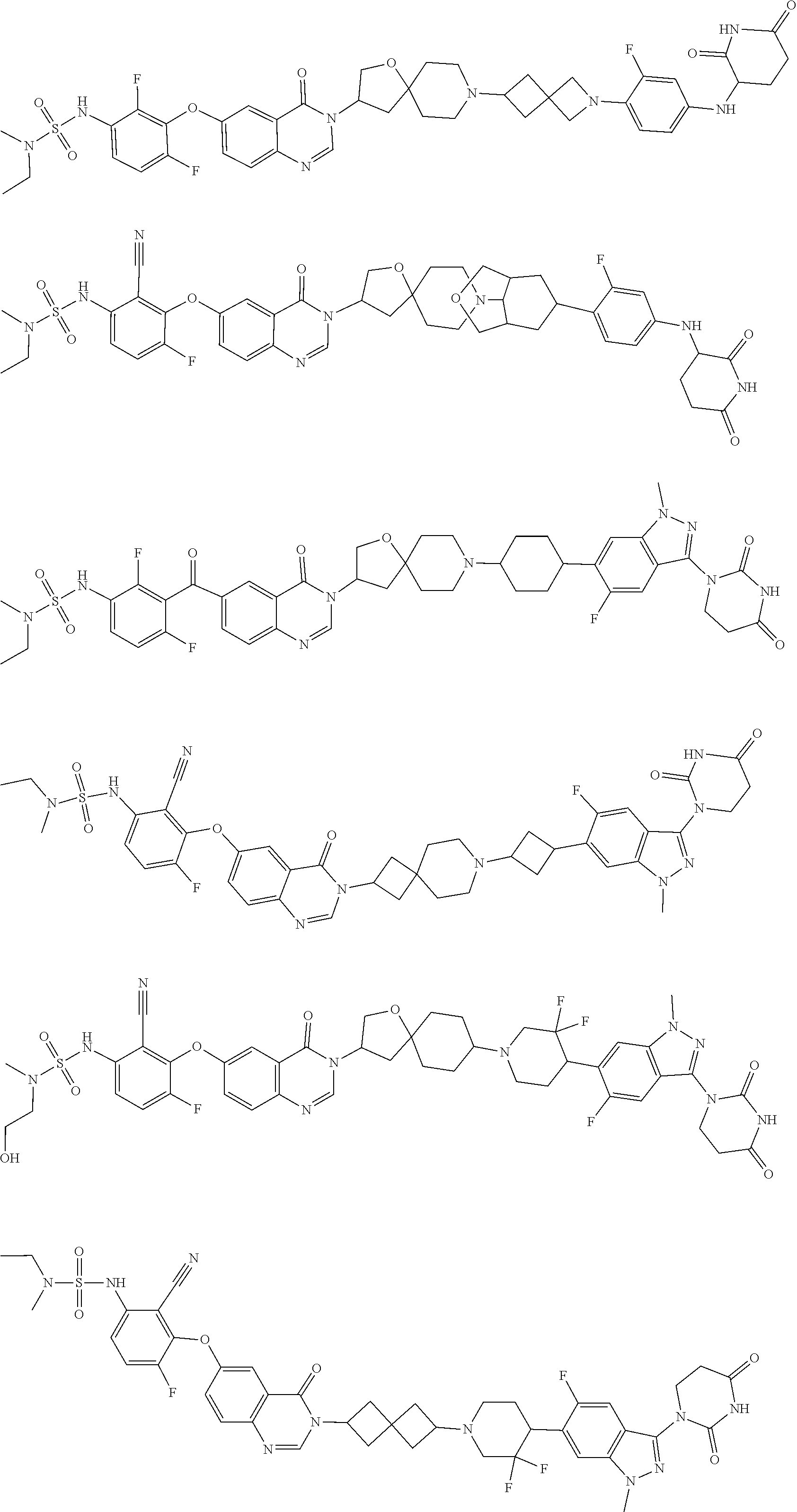

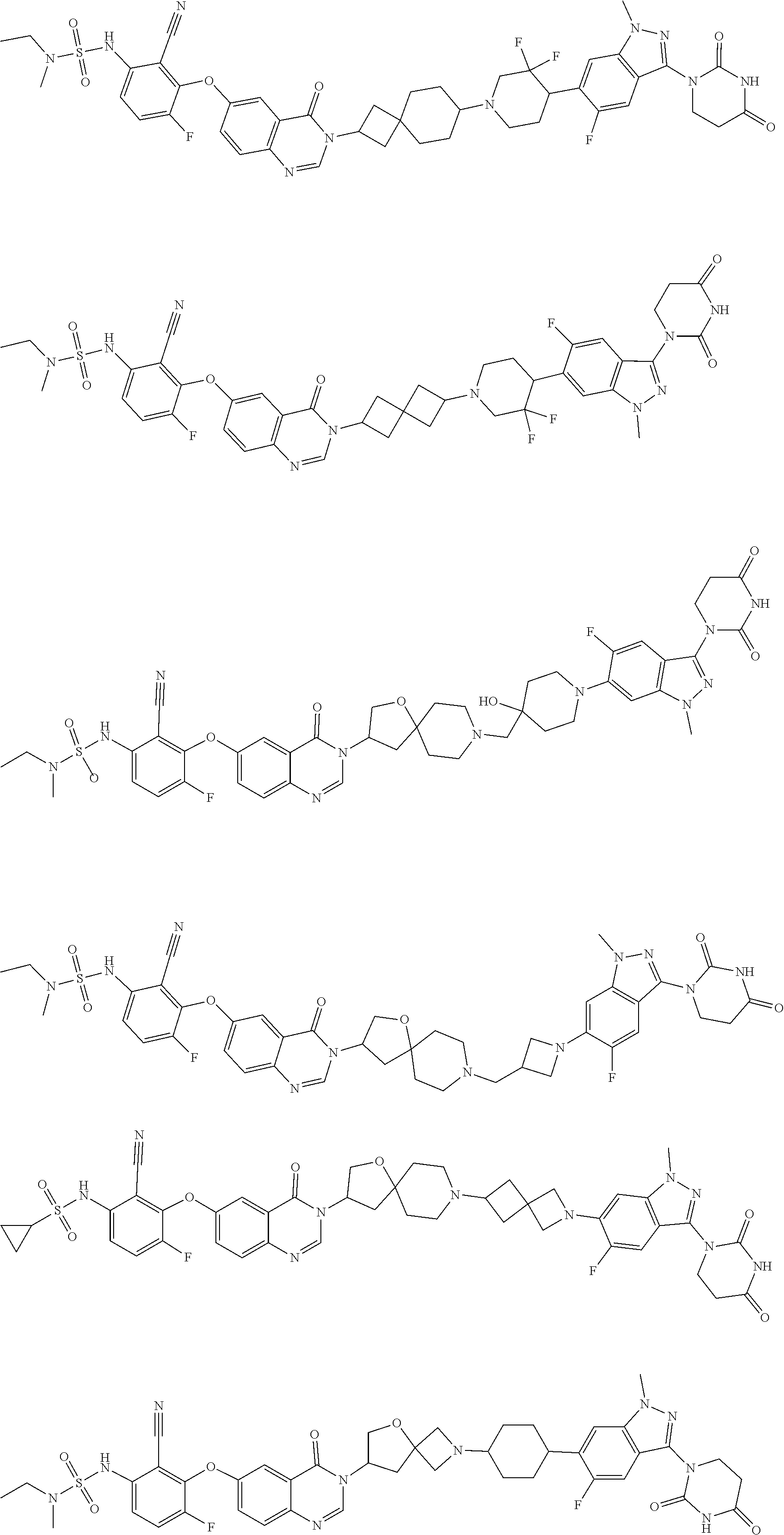

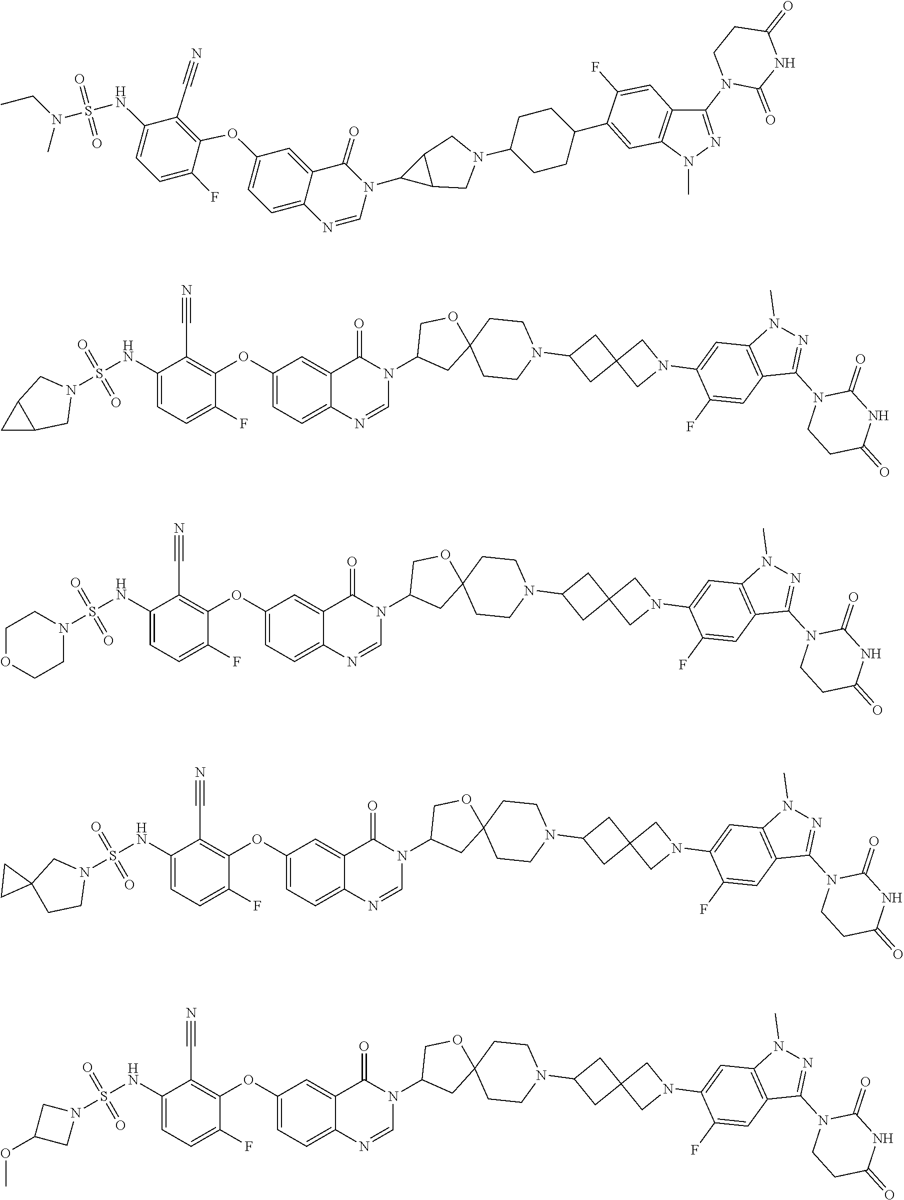

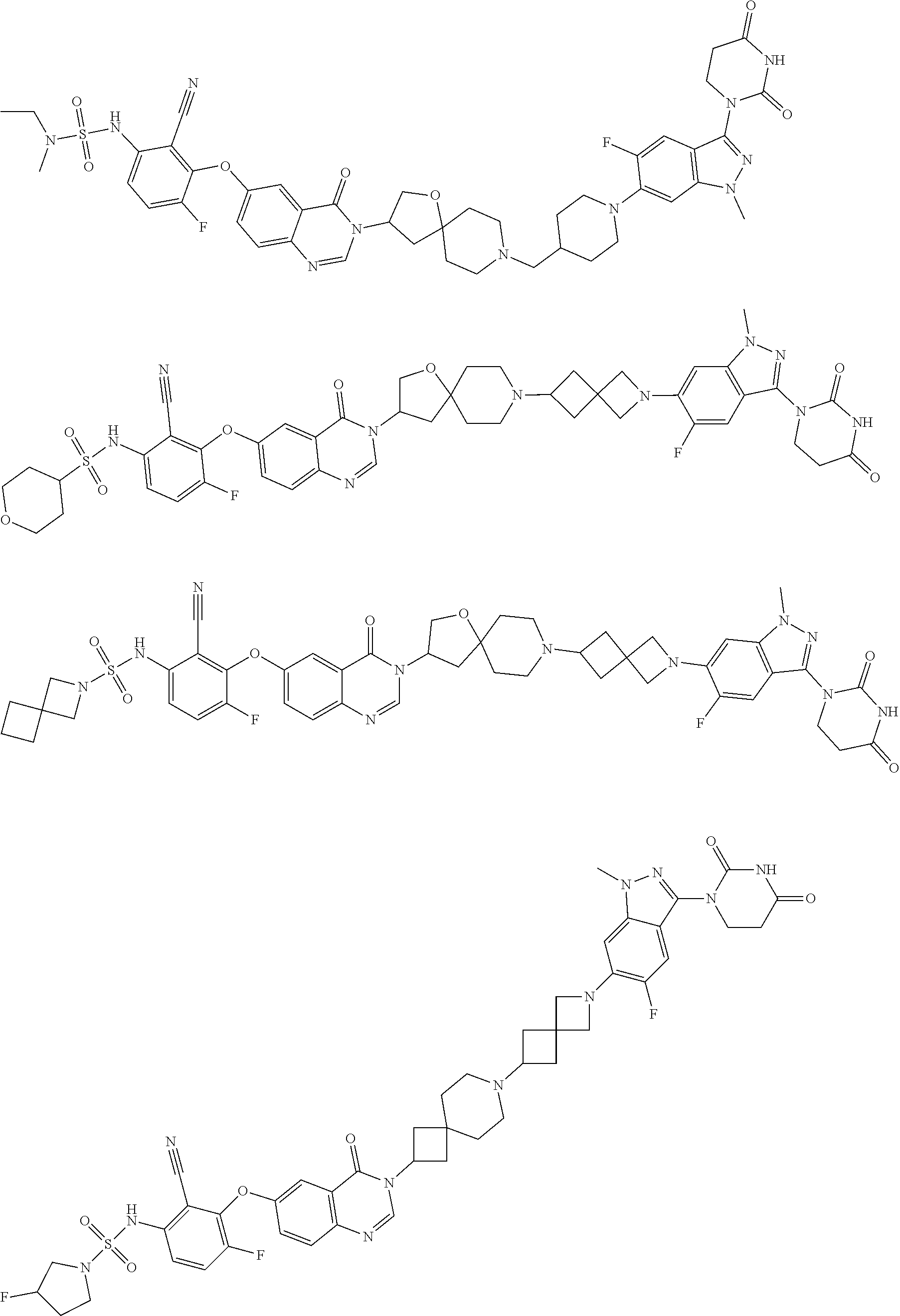 [SMILES: CCN(C)S(=O)(=O)Nc1ccc(F)c(Oc2ccc3ncn(C4COC5(CCN(CC6CCN(c7cc8c(cc7F)c(N7CCC(=O)NC7=O)nn8C)CC6)CC5)C4)c(=O)c3c2)c1C#N ; Cn1nc(N2CCC(=O)NC2=O)c2cc(F)c(N3CC4(CC(N5CCC6(CC5)CC(n5cnc7ccc(Oc8c(F)ccc(NS(=O)(=O)C9CCOCC9)c8C#N)cc7c5=O)CO6)C4)C3)cc21 ; Cn1nc(N2CCC(=O)NC2=O)c2cc(F)c(N3CC4(CC(N5CCC6(CC5)CC(n5cnc7ccc(Oc8c(F)ccc(NS(=O)(=O)N9CC%10(CCC%10)C9)c8C#N)cc7c5=O)CO6)C4)C3)cc21 ; Cn1nc(N2CCC(=O)NC2=O)c2cc(F)c(N3CC4(CC(N5CCC6(CC5)CC(n5cnc7ccc(Oc8c(F)ccc(NS(=O)(=O)N9CCC(F)C9)c8C#N)cc7c5=O)C6)C4)C3)cc21]

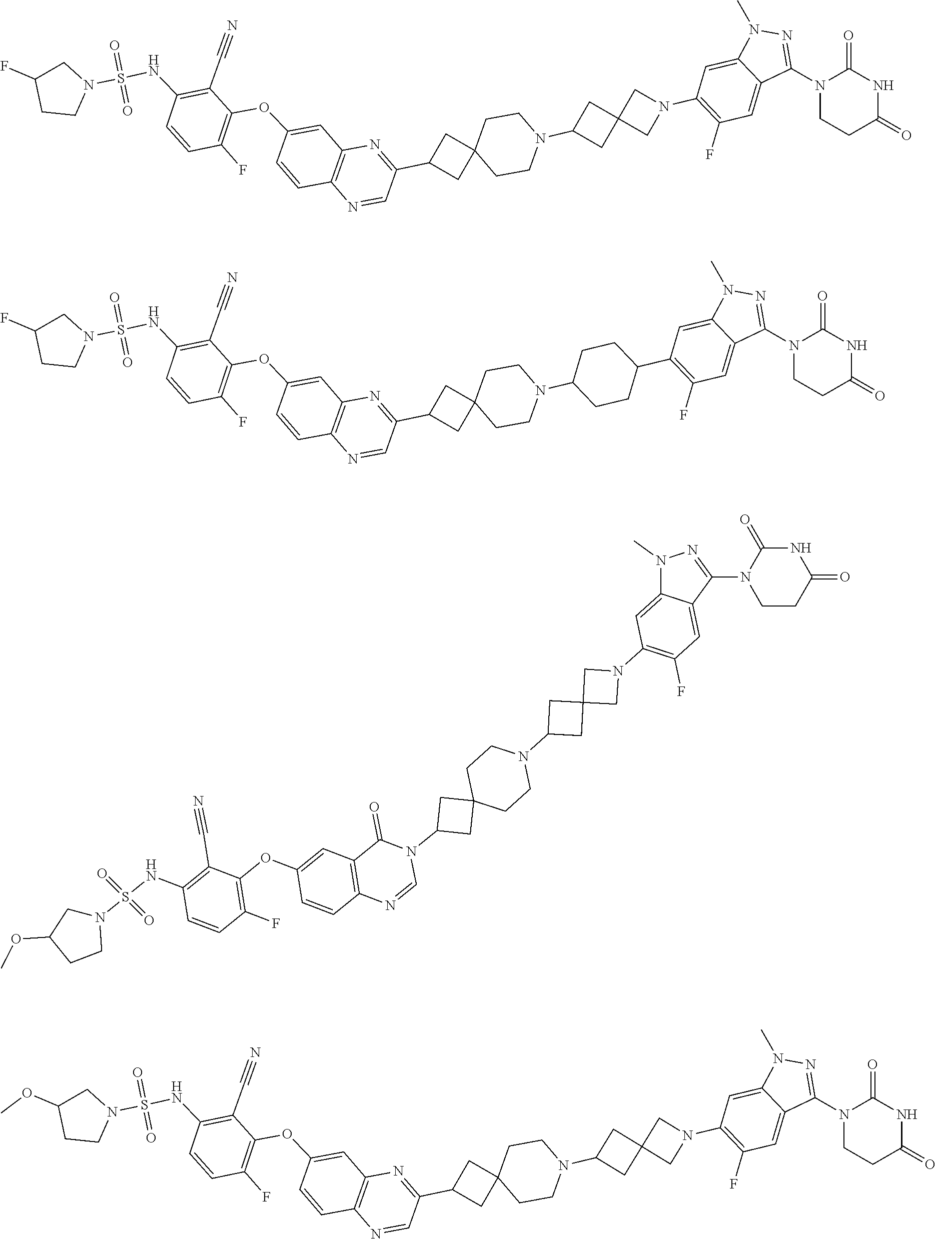 [SMILES: Cn1nc(N2CCC(=O)NC2=O)c2cc(F)c(N3CC4(CC(N5CCC6(CC5)CC(c5cnc7ccc(Oc8c(F)ccc(NS(=O)(=O)N9CCC(F)C9)c8C#N)cc7n5)C6)C4)C3)cc21 ; Cn1nc(N2CCC(=O)NC2=O)c2cc(F)c(C3CCC(N4CCC5(CC4)CC(c4cnc6ccc(Oc7c(F)ccc(NS(=O)(=O)N8CCC(F)C8)c7C#N)cc6n4)C5)CC3)cc21 ; COC1CCN(S(=O)(=O)Nc2ccc(F)c(Oc3ccc4ncn(C5CC6(CCN(C7CC8(C7)CN(c7cc9c(cc7F)c(N7CCC(=O)NC7=O)nn9C)C8)CC6)C5)c(=O)c4c3)c2C#N)C1 ; COC1CCN(S(=O)(=O)Nc2ccc(F)c(Oc3ccc4ncc(C5CC6(CCN(C7CC8(C7)CN(c7cc9c(cc7F)c(N7CCC(=O)NC7=O)nn9C)C8)CC6)C5)nc4c3)c2C#N)C1]

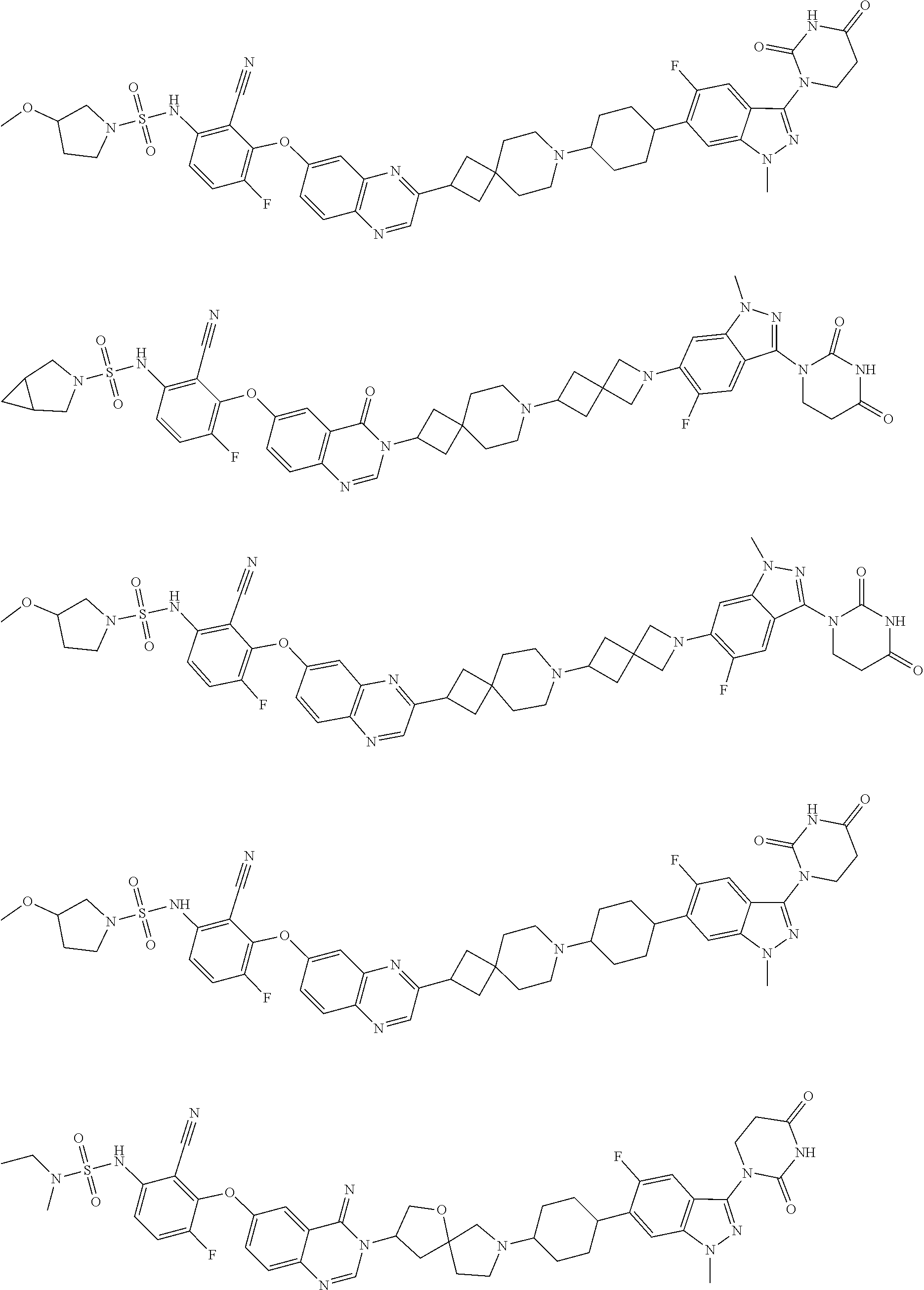

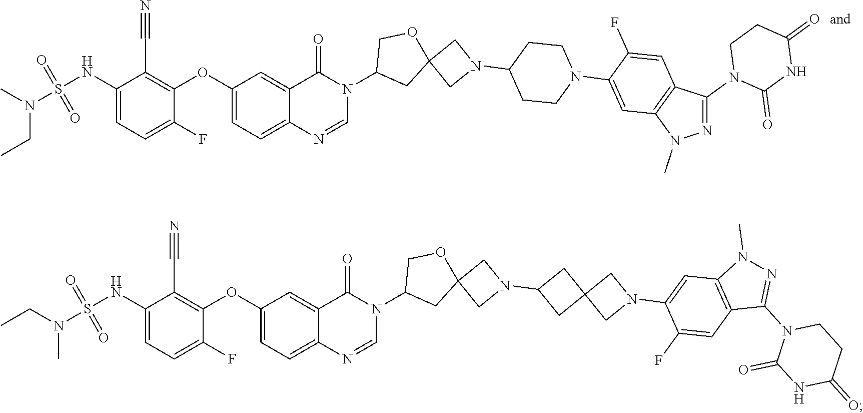 [SMILES: CCN(C)S(=O)(=O)Nc1ccc(F)c(Oc2ccc3ncn(C4COC5(C4)CN(C4CCN(c6cc7c(cc6F)c(N6CCC(=O)NC6=O)nn7C)CC4)C5)c(=O)c3c2)c1C#N ; "and" ; CCN(C)S(=O)(=O)Nc1ccc(F)c(Oc2ccc3ncn(C4COC5(C4)CN(C4CC6(C4)CN(c4cc7c(cc4F)c(N4CCC(=O)NC4=O)nn7C)C6)C5)c(=O)c3c2)c1C#N]

or a pharmaceutically acceptable salt thereof.

### Methods of Treatment

A compound of the present invention, its pharmaceutically acceptable salt or pharmaceutical composition thereof can be used in an effective amount to treat a patient with a disorder mediated by a mutant BRAF. In certain aspects the disorder mediated by a mutant BRAF is a cancer. In certain embodiments the mutant BRAY mediated cancer has metastasized to the brain or CNS.

BRAF is a serine/threonine protein kinase that is a member of the signal transduction protein kinases. BRAY V600X mutations, in particular BRAF V600E/K mutations are often observed in a variety of human tumors including melanoma, thyroid cancer, colorectal cancer, lung cancer and others. Non-limiting examples of V600X mutations include V600E, V600K, V600R, V600D, and V600N. Despite the therapeutic benefits exerted by available BRAF inhibitors in the clinic in many of these indications, the duration of the antitumor response to these drugs is limited by the acquisition of drug resistance.

The BRAF protein presents a mechanism for signaling propagation that requires protein homo-dimerization (BRAF-BRAF) or hetero-dimerization with other RAF proteins (BRAF-RAF1 or BRAF-ARAF). When BRAF is mutated, as observed in oncological indications with BRAF V600X substitution, BRAF signaling becomes independent from the generation of homodimers and/or heterodimers. In this context, the kinase becomes hyperactivated as a monomeric protein and drives cellular proliferative signals.

Because classic inhibitors only block BRAF activity in its monomeric form and are ineffective on BRAF homodimers or heterodimers, it is not surprising that many BRAF-resistance inducing mechanisms act by restoring RAF homodimerization and heterodimerization mediated signaling.

Targeted protein degradation induces target ubiquitination by recruiting an E3 ligase thus promoting proteasome-mediated disruption of the engaged target. The degradation of BRAF through targeted degradation offers an advantage over conventional inhibition since it eliminates scaffolding activities of BRAF V600E/K and particularly, induces BRAF protein elimination. This activity prevents the dimerization-mediated mechanisms of resistance.

BRAF protein abrogation may represent a strategy to delay the onset of resistance acquisition as well as potentially targeting tumors that acquired resistance to available inhibitors. This observation offers novel therapeutic opportunities in the treatment of BRAF V600X mutated tumors like melanoma, colorectal cancer, and lung cancer.

Another aspect of the present invention provides a compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing cancer in a patient in need thereof, wherein there is a need of BRAF inhibition for the treatment or prevention of cancer.

In certain aspects, a compound of the present invention is used to treat a BRAF mediated cancer, wherein the BRAF has mutated from the wild type. There are a number of possibilities for BRAF mutations. In certain non-limiting embodiments, the mutation is a Class I mutation, a Class II mutation, or a Class III mutation, or any combination thereof. Non-limiting examples of Class I mutations include V600 mutations such as V600E, V600K, V600R, V600D, and V600N. Non-limiting examples of Class II mutations include G469A, G469V, G469L, G469R, L597Q, and K601E. Non-limiting examples of Class III mutations include G466A, G466E, G466R, G466V, S467L, G469E, N581I, D594E, D594G, and D594N.

In certain embodiments a compound of the present invention treats a BRAF mutant mediated disorder wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E.

In certain embodiments the BRAF mutation is an exon 11 mutation.

In certain embodiments the BRAF mutation is an exon 15 mutation.

In certain embodiments the BRAF mutation is a G464 mutation.

In certain embodiments the BRAF mutation is a G466 mutation.

In certain embodiments the BRAF mutation is a G466R mutation.

In certain embodiments the BRAF mutation is a G466E mutation.

In certain embodiments the BRAF mutation is a G469 mutation.

In certain embodiments the BRAF mutation is a G469E mutation.

In certain embodiments the BRAF mutation is a D594 mutation.

In certain embodiments the BRAF mutation is a D594A mutation.

In certain embodiments the BRAF mutation is a L597 mutation.

In certain embodiments the BRAF mutation is a L597R mutation.

In certain embodiments the BRAF mutation is a L597S mutation.

In certain embodiments the BRAF mutation is a L597Q mutation.

In certain embodiments the BRAF mutation is a V600 mutation.

In certain embodiments the BRAF mutation is a V600E mutation.

In certain embodiments the BRAF mutation is a V600K mutation.

In certain embodiments the BRAF mutation is a V600R mutation.

In certain embodiments the BRAF mutation is a V600Dmutation.

In certain embodiments the BRAF mutation is a K601 mutation.

In certain embodiments the BRAF mutation is a K601E mutation.

In certain embodiments the BRAF mutation is a K601N mutation.

In certain embodiments the disorder is cancer that has metastasized to the brain or CNS and the BRAF mutation is an exon 11 mutation.

In certain embodiments the disorder is cancer that has metastasized to the brain or CNS and the BRAF mutation is an exon 15 mutation.

In certain embodiments the disorder is cancer that has metastasized to the brain or CNS and the BRAF mutation is a G464 mutation.

In certain embodiments the disorder is cancer that has metastasized to the brain or CNS and the BRAF mutation is a G466 mutation.

In certain embodiments the disorder is cancer that has metastasized to the brain or CNS and the BRAF mutation is a G466R mutation.

In certain embodiments the disorder is cancer that has metastasized to the brain or CNS and the BRAF mutation is a G466E mutation.

In certain embodiments the disorder is cancer that has metastasized to the brain or CNS and the BRAF mutation is a G469 mutation.

In certain embodiments the disorder is cancer that has metastasized to the brain or CNS and the BRAF mutation is a G469E mutation.

In certain embodiments the disorder is cancer that has metastasized to the brain or CNS and the BRAF mutation is a D594 mutation.

In certain embodiments the disorder is cancer that has metastasized to the brain or CNS and the BRAF mutation is a D594A mutation.

In certain embodiments the disorder is cancer that has metastasized to the brain or CNS and the BRAF mutation is a L597 mutation.

In certain embodiments the disorder is cancer that has metastasized to the brain or CNS and the BRAF mutation is a L597R mutation.

In certain embodiments the disorder is cancer that has metastasized to the brain or CNS and the BRAF mutation is a L597S mutation.

In certain embodiments the disorder is cancer that has metastasized to the brain or CNS and the BRAF mutation is a L597Q mutation.

In certain embodiments the disorder is cancer that has metastasized to the brain or CNS and the BRAF mutation is a V600 mutation.

In certain embodiments the disorder is cancer that has metastasized to the brain or CNS and the BRAF mutation is a V600E mutation.

In certain embodiments the disorder is cancer that has metastasized to the brain or CNS and the BRAF mutation is a V600K mutation.

In certain embodiments the disorder is cancer that has metastasized to the brain or CNS and the BRAF mutation is a V600R mutation.

In certain embodiments the disorder is cancer that has metastasized to the brain or CNS and the BRAF mutation is a V600Dmutation.

In certain embodiments the disorder is cancer that has metastasized to the brain or CNS and the BRAF mutation is a K601 mutation.

In certain embodiments the disorder is cancer that has metastasized to the brain or CNS and the BRAF mutation is a K601E mutation.

In certain embodiments the disorder is cancer that has metastasized to the brain or CNS and the BRAF mutation is a K601N mutation.

In certain embodiments the cancer has metastasized to the brain. In certain embodiments the cancer has metastasized to the CNS.

In certain embodiments a compound of the present invention treats a BRAF mutant mediated disorder wherein the mutation is a splice variant, for example p61-BRAF^{V600E}. In certain embodiments the BRAF mutation is a splice variant, for example p61-BRAF^{V600E}, and the cancer has metastasized to the brain or CNS.

In certain embodiments a compound of the present invention is used to treat a disorder that is mediated by two or more mutant proteins, for example a cancer mediated by a BRAF^{V600E}/NRAS^{Q61K} double mutant. Non-limiting examples of double mutant cancers include colorectal cancer which is mediated by a BRAF mutation, for example BRAF^{V600E} and a mutation of NRAS, MEK1, or PI3K, for example, BRAF^{V600E}/PIK3CA^{H1047R} or BRAF^{V600E}/PIK3CA^{P449T}. In certain embodiments the cancer that is mediated by two or more mutant proteins has metastasized to the brain or CNS.

In certain embodiments, a compound of the present invention is used to treat a cancer that is resistant to at least one BRAF inhibitor, for example a cancer that is resistant to or has acquired resistance to a BRAF inhibitor selected from dabrafenib, vemurafenib, and encorafenib. In certain aspects the cancer that is resistant to treatment with a BRAF inhibitor has a RAF protein homo-dimerization or hetero-dimerization promoting mutation. For example, in certain embodiments a compound of the present invention is used to treat a cancer with one or more mutations that promote RAF dimerization. In certain embodiments the cancer with one or more RAF dimerization promoting mutations is resistant to treatment with a BRAF inhibitor for example dabrafenib, vemurafenib, or encorafenib. In certain aspects the mutation is a BRAF homo-dimerization promoting mutation. In certain embodiments a compound of the present invention is used to treat a BRAY mutant mediated cancer that has metastasized to the brain or CNS wherein the cancer has one or more RAF dimerization promoting mutations.

In certain embodiments a compound described herein is used to treat a cancer that has developed an escape mutation such as BRAF V600E/NRAS^{Q61K} double mutant cancer.

In certain embodiments a compound described herein is used to treat melanoma. For example, in certain embodiments a compound of the present invention is used to treat a melanoma that has metastasized to the brain or CNS. In certain embodiments a compound described herein is used to treat colorectal cancer. For example, in certain embodiments a compound of the present invention is used to treat a colorectal cancer that has metastasized to the brain or CNS.

Non-limiting examples of melanoma include nonacral cutaneous melanoma, acral melanoma, mucosal melanoma, uveal melanoma, and leptomeningeal melanoma, each of which can be primary or metastatic.

In certain embodiments a compound of the present invention is used to treat triple negative breast cancer, for example triple negative breast cancer with a G464V BRAF mutant.

In certain embodiments a compound of the present invention is used to treat lung cancer, for example lung adenocarcinoma with a G466V BRAF mutant.

In certain embodiments a compound of the present invention is used to treat melanoma with a V600 BRAF mutant.

In certain aspects, a compound of the invention is used to treat a BRAF mediated cancer, wherein the BRAF has mutated from the wild type. There are a number of possibilities for BRAF mutations. In certain non-limiting embodiments, the mutation is a Class I mutation, a Class II mutation, or a Class III mutation, or any combination thereof. Non-limiting examples of Class I mutations include V600 mutations such as V600E, V600K, V600R, V600D, and V600N. Non-limiting examples of Class II mutations include G469A, G469V, G469L, G469R, L597Q, and K601E. Non-limiting examples of Class III mutations include G466A, G466E, G466R, G466V, S467L, G469E, N581I, D594E, D594G, and D594N.

In certain embodiments a compound of the invention treats a BRAF mutant mediated disorder wherein the mutation is not a Class I, Class II, or Class III mutation. Non-limiting examples of mutations include G464I, G464R, N581T, L584F, E586K, G593D, G596C, L597R, L597S, S605I, S607F, N684T, E26A, V130M, L745L, and D284E.

In certain embodiments a compound of the invention treats a BRAF mutant mediated disorder wherein the mutation is a splice variant, for example p61-BRAF^{V600E}.

In certain embodiments a compound of the invention is used to treat a disorder that is mediated by two or more mutant proteins, for example a cancer mediated by a BRAF^{V600E}/NRAS^{Q61K} double mutant.

In certain embodiments, a compound of the invention is used to treat a cancer that is resistant to at least one BRAF inhibitor, for example a cancer that is resistant to or has acquired resistance to a BRAF inhibitor selected from dabrafenib, vemurafenib and encorafenib.

In certain embodiments a compound of the invention is used to treat a cancer that has developed an escape mutation such as BRAF V600E NRAS^{Q61K} double mutant cancer.

In certain embodiments a compound of the invention is used to treat melanoma.

In certain embodiments a compound of the invention is used to treat triple negative breast cancer, for example triple negative breast cancer with a G464V BRAF mutant.

In certain embodiments a compound of the invention is used to treat lung cancer, for example lung adenocarcinoma with a G466V BRAF mutant.

In certain embodiments a compound of the invention is used to treat melanoma with a V600 BRAF mutant.

In certain embodiments a compound of the invention is used to treat cholangiocarcinoma.

In certain embodiments a compound of the invention is used to treat erdeheim-chester disease.

In certain embodiments a compound of the invention is used to treat langerhans histiocytosis.

In certain embodiments a compound of the invention is used to treat ganglioglioma.

In certain embodiments a compound of the invention is used to treat glioma.

In certain embodiments a compound of the invention is used to treat GIST.

In certain embodiments a compound of the invention is used to treat glioblastoma.

In certain embodiments a compound of the invention is used to treat hairy cell leukemia.

In certain embodiments a compound of the invention is used to treat multiple myeloma.

In certain embodiments a compound of the invention is used to treat non-small-cell lung cancer.

In certain embodiments a compound of the invention is used to treat ovarian cancer.

In certain embodiments a compound of the invention is used to treat pilomyxoid astrocytoma.

In certain embodiments a compound of the invention is used to treat anaplastic pleomorphic xanthoastrocytoma.

In certain embodiments a compound of the invention is used to treat astrocytoma.

In certain embodiments a compound of the invention is used to treat thyroid cancer.

In certain embodiments a compound of the invention is used to treat papillary thyroid cancer.

In certain embodiments a compound of the invention is used to treat anaplastic thyroid cancer.

In certain embodiments a compound of the invention is used to treat pancreatic cancer.

In certain embodiments a compound of the invention is used to treat thoracic clear cell sarcoma.

In certain embodiments a compound of the invention is used to treat salivary gland cancer.

In certain embodiments a compound of the invention is used to treat colorectal cancer.

In certain embodiments a compound of the invention is used to treat microsatellite stable colorectal cancer.

In certain embodiments a compound of the present invention is used to treat a disorder selected from cholangiocarcinoma, erdeheim-chester disease, langerhans histiocytosis, ganglioglioma, glioma, GIST, glioblastoma, hairy cell leukemia, metanephric adenoma, multiple myeloma, lung cancer, non-small-cell lung cancer, ovarian cancer, pilomyxoid astrocytoma, anaplastic pleomorphic xanthoastrocytoma, astrocytoma, thyroid cancer, papillary thyroid cancer, anaplastic thyroid cancer, pancreatic cancer, thoracic clear cell sarcoma, salivary gland cancer, colorectal cancer, and microsatellite stable colorectal cancer.

Another aspect of the present invention provides a method of treating or preventing a proliferative disease. The method comprises administering an effective amount of a pharmaceutical composition comprising a compound as described herein, or an enantiomer, diastereomer, or stereoisomer thereof, or pharmaceutically acceptable salt, hydrate, or solvate thereof and optionally a pharmaceutically acceptable carrier to a patient in need thereof.

In certain aspects to “treat” a disease as the term is used herein, means to reduce the frequency or severity of at least one sign or symptom of a disease or disorder experienced by a patient (i.e., palliative treatment) or to decrease a cause or effect of the disease or disorder (i.e., disease-modifying treatment).

In some aspects a compound of the present invention is administered parenterally. Parenteral” administration of a pharmaceutical composition includes, e.g., subcutaneous (s.c.), intravenous (i.v.), intramuscular (i.m.), intrasternal injection, or infusion techniques.

In certain embodiments, the disease or disorder is cancer or a proliferation disease.

In certain embodiments, the BRAF mediated disorder is an abnormal cell proliferation, including, but not limited to, a solid or hematological cancer.

In certain embodiments, the hematological cancer is acute myelogenous leukemia (AML), acute lymphoblastic leukemia (ALL), lymphoblastic T-cell leukemia, chronic myelogenous leukemia (CML), chronic lymphocytic leukemia (CLL), hairy-cell leukemia, chronic neutrophilic leukemia (CNL), acute lymphoblastic T-cell leukemia, acute monocytic leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukemia (MLL), erythroleukemia, malignant lymphoma, Hodgkin's lymphoma, non-Hodgkin's lymphoma, lymphoblastic T-cell lymphoma, Burkitt's lymphoma, follicular lymphoma, B cell acute lymphoblastic leukemia, diffuse large B cell lymphoma, Myc and B-Cell Leukemia (BCL)2 and/or BCL6 rearrangements/overexpression [double- and triple-hit lymphoma], myelodysplastic/myeloproliferative neoplasm, mantle cell lymphoma including bortezomib resistant mantle cell lymphoma.

Solid tumors that can be treated with the compounds described herein include, but are not limited to lung cancers, including small cell lung cancer (SCLC) and non-small cell lung cancer (NSCLC), breast cancers including inflammatory breast cancer, ER-positive breast cancer including tamoxifen resistant ER-positive breast cancer, and triple negative breast cancer, colon cancers, midline carcinomas, liver cancers, renal cancers, prostate cancers including castrate resistant prostate cancer (CRPC), brain cancers including gliomas, glioblastomas, neuroblastoma, and medulloblastoma including MYC-amplified medulloblastoma, colorectal cancers, Wilm's tumor, Ewing's sarcoma, rhabdomyosarcomas, ependymomas, head and neck cancers, melanomas, squamous cell carcinomas, ovarian cancers, pancreatic cancers including pancreatic ductal adenocarcinomas (PDAC) and pancreatic neuroendocrine tumors (PanNET), osteosarcomas, giant cell tumors of bone, thyroid cancers, bladder cancers, urothelial cancers, vulval cancers, cervical cancers, endometrial cancers, mesotheliomas, esophageal cancers, salivary gland cancers, gastric cancers, nasopharyngeal cancers, buccal cancers, cancers of the mouth, GIST (gastrointestinal stromal tumors), NUT-midline carcinomas, testicular cancers, squamous cell carcinomas, hepatocellular carcinomas (HCC), MYCN driven solid tumors, and NUT midline carcinomas (NMC).

In further embodiments, the disease or disorder is sarcoma of the bones, muscles, tendons, cartilage, nerves, fat, or blood vessels.

In further embodiments, the disease or disorder is soft tissue sarcoma, bone sarcoma, or osteosarcoma.

In further embodiments, the disease or disorder is angiosarcoma, fibrosarcoma, liposarcoma, leiomyosarcoma, Kaposi's sarcoma, osteosarcoma, gastrointestinal stromal tumor, synovial sarcoma, pleomorphic sarcoma, chondrosarcoma, Ewing's sarcoma, reticulum cell sarcoma, hemangiosarcoma, botryoid sarcoma, rhabdomyosarcoma, or embryonal rhabdomyosarcoma.

In certain embodiments the disorder is a bone, muscle, tendon, cartilage, nerve, fat, or blood vessel sarcoma.

In other embodiments, the pharmaceutical composition comprising the compound as described herein and the additional therapeutic agent are administered simultaneously or sequentially.

In other embodiments, the disease or disorder is cancer. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, solid tumors, hematological cancers or solid cancers.

One aspect of this application provides compounds that are useful for the treatment of diseases, disorders, and conditions characterized by excessive or abnormal cell proliferation. Such diseases include, but are not limited to, a proliferative or hyperproliferative disease. Examples of proliferative and hyperproliferative diseases include, without limitation, cancer. The term “cancer” includes, but is not limited to, the following cancers: breast; ovary; cervix; prostate; testis, genitourinary tract; esophagus; larynx, glioblastoma; neuroblastoma; stomach; skin, keratoacanthoma; lung, epidermoid carcinoma, large cell carcinoma, small cell carcinoma, lung adenocarcinoma; bone; colon; colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colorectum, large intestine, rectum, brain and central nervous system; chronic myeloid leukemia (CML), and leukemia. The term “cancer” includes, but is not limited to, the following cancers: myeloma, lymphoma, or a cancer selected from gastric, renal, or and the following cancers: head and neck, oropharyngeal, non-small cell lung cancer (NSCLC), endometrial, hepatocarcinoma, non-Hodgkin's lymphoma, and pulmonary.

In certain aspects the term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotropic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers, such as oral, laryngeal, nasopharyngeal and esophageal, genitourinary cancers, such as prostate, bladder, renal, uterine, ovarian, testicular, lung cancer, such as small-cell and non-small cell, breast cancer, pancreatic cancer, melanoma and other skin cancers, stomach cancer, brain tumors, tumors related to Gorlin's syndrome, such as medulloblastoma or meningioma, and liver cancer.

Additional exemplary forms of cancer include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidal melanoma, seminoma, rhabdomyosarcoma, craniopharyngioma, osteosarcoma, chondrosarcoma, myosarcoma, liposarcoma, fibrosarcoma, Ewing sarcoma, and plasmacytoma. In one aspect of the application, the present application provides for the use of one or more compound as described herein, in the manufacture of a medicament for the treatment of cancer, including without limitation the various types of cancer disclosed herein.

In some embodiments, the compounds of this application are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compound as described herein is useful for treating hematopoietic disorders, in particular, acute-myelogenous leukemia (AML), chronic-myelogenous leukemia (CML), acute-promyelocytic leukemia, and acute lymphocytic leukemia (ALL).

In certain embodiments, a compound or it's corresponding pharmaceutically acceptable salt, or isotopic derivative, as described herein can be used in an effective amount to treat a host, for example a human, with a lymphoma or lymphocytic or myelocytic proliferation disorder or abnormality. For example, a compound as described herein can be administered to a host suffering from a Hodgkin's Lymphoma or a Non-Hodgkin's Lymphoma. For example, the host can be suffering from a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); diffuse small-cleaved cell lymphoma (DSCCL); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; Langerhans cell histiocytosis; or Waldenstrom's Macroglobulinemia.

In another embodiment, a compound or it's corresponding pharmaceutically acceptable salt, or isotopic derivative, as described herein can be used in an effective amount to treat a patient, for example a human, with a Hodgkin's lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin's Lymphoma; or Nodular Lymphocyte Predominant HL.

This application further embraces the treatment or prevention of cell proliferative disorders such as hyperplasias, dysplasias and pre-cancerous lesions. Dysplasia is the earliest form of pre-cancerous lesion recognizable in a biopsy by a pathologist. The compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

In accordance with the foregoing, the present application further provides a method for preventing or treating any of the diseases or disorders described above in a patient in need of such treatment, which method comprises administering to said patient a therapeutically effective amount of a compound as described herein, or an enantiomer, diastereomer, or stereoisomer thereof, or pharmaceutically acceptable salt, hydrate, or solvate thereof. For any of the above uses, the required dosage will vary depending on the mode of administration, the particular condition to be treated and the effect desired.

### Combination Therapy

A compound described herein, or its pharmaceutically acceptable salt or pharmaceutical composition can be used in an effective amount alone or in combination with another compound of the present invention or another bioactive agent or second therapeutic agent to treat a patient such as a human with a mutant BRAF mediated disorder, including but not limited to those described herein.

The term “bioactive agent” or “additional active agent” is used to describe an agent, other than the selected compound according to the present invention, which can be used in combination or alternation with a compound of the present invention to achieve a desired result of therapy. In certain embodiments, the compound of the present invention and the bioactive agent are administered in a manner that they are active in vivo during overlapping time periods, for example, have time-period overlapping Cmax, Tmax, AUC or another pharmacokinetic parameter. In another embodiment, the compound of the present invention and the bioactive agent are administered to a patient in need thereof that do not have overlapping pharmacokinetic parameter, however, one has a therapeutic impact on the therapeutic efficacy of the other.

In some embodiments, a selected compound provided herein, or its pharmaceutically acceptable salt is used in combination with another BRAF inhibitor such as sorafenib, vemurafenib (ZELBORAF®), dabrafenib (TAFINLAR®) or encorafenib (BRAFTOVI©).

In certain embodiments, the bioactive agent is a MEK inhibitor. MEK inhibitors are well known, and include, for example, trametinib/GSK1120212 (N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)-N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2y1)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088.

In certain embodiments the MEK inhibitor is trametinib.

In certain embodiments a compound of the present invention is used in combination with cetuximab or trametinib to treat colorectal cancer. In certain embodiments a compound of the present invention is used in combination with cetuximab and BYL719 to treat colorectal cancer. In certain embodiments a compound of the present invention is used in combination with cetuximab and irinotecan to treat colorectal cancer.

In certain embodiments a compound of the invention is used in combination with cetuximab or trametinib to treat colorectal cancer. In certain embodiments a compound of the invention is used in combination with cetuximab and BYL719 to treat colorectal cancer. In certain embodiments a compound of the invention is used in combination with cetuximab and irinotecan to treat colorectal cancer.

In certain embodiments the bioactive agent is a SHP2 inhibitor. In certain embodiments the SHP2 inhibitor is SHP099.

In certain embodiments the bioactive agent is a RAF inhibitor. Non-limiting examples of Raf inhibitors include, for example, vemurafenib (N-[3-[[5-(4-chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide;4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), sorafenib N-oxide (4-[4-[[[[4-Chloro-3(trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (encorafenib (BRAFTOVI©)).

In certain embodiments the RAF inhibitor is encorafenib.

In certain embodiments the RAF inhibitor is vemurafenib.

In certain embodiments the RAF inhibitor is dabrafenib.

In certain embodiments, the bioactive agent is an EGFR inhibitor, including, for example gefitinib (IRESSA®), erlotinib (TARCEVA®), lapatinib (TYKERB®), osimertinib (TAGRISSO®), neratinib (NERLYNX®), vandetanib (CAPRELSA®), dacomitinib (VIZIMPRO®), rociletinib (XEGAFRI™), afatinib (GLOTRIF®, GIOTRIFF™, AFANIX™) lazertinib, or nazartib. In other embodiments the bioactive agent is an EGFR degrader.

Additional examples of EGFR inhibitors include rociletinib (CO-1686), olmutinib (OLITA™), naquotinib (ASP8273), nazartinib (EGF816), PF-06747775, icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, dacomitinib (PF-00299804; Pfizer), brigatinib (ALUNBRIG®), lorlatinib, and PF-06747775 (PF7775).

In certain embodiments, the bioactive agent is a first-generation EGFR inhibitor such as erlotinib, gefitinib, or lapatinib. In certain embodiments, the bioactive agent is a second-generation EGFR inhibitor such as afatinib and/or dacomitinib. In certain embodiments, the bioactive agent is a third-generation EGFR inhibitor such as osimertinib.

In certain embodiments a compound of the present invention is administered to a patient in need thereof in combination with osimertinib.

In certain embodiments a compound of the present invention is administered to a patient in need thereof in combination with rociletinib.

In certain embodiments a compound of the present invention is administered to a patient in need thereof in combination with avitinib.

In certain embodiments a compound of the present invention is administered to a patient in need thereof in combination with lazertinib.

In certain embodiments a compound of the present invention is administered to a patient in need thereof in combination with nazartinib.

In certain embodiments a compound of the present invention is administered to a patient in need thereof in combination with an EGFR antibody, for example, cetuximab, panitumumab, or necitumumab.

In certain embodiments a compound of the present invention is administered to a patient in need thereof in combination with cetuximab.

In certain embodiments a compound of the present invention is administered to a patient in need thereof in combination with panitumumab.

In certain embodiments a compound of the present invention is administered to a patient in need thereof in combination with necitumumab.

In one aspect of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or another inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (OPDIVO®), pembrolizumab (KEYTRUDA®), pidilizumab, AMP-224 (AstraZeneca and MedImmune), PF-06801591 (Pfizer), MEDIO680 (AstraZeneca), PDR001 (Novartis), REGN2810 (Regeneron), SHR-12-1 (Jiangsu Hengrui Medicine Company and Incyte Corporation), TSR-042 (GlaxoSmithKline plc), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (TECENTRIQ®), durvalumab (AstraZeneca and MedImmune), KN035 (Alphamab Co. Ltd.), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors include, but are not limited to, BMS-986016 (Bristol-Myers Squibb), GSK2831781 (GlaxoSmithKline plc), IMP321 (Prima BioMed), LAG525 (Novartis), and the dual PD-1 and LAG-3 inhibitor MGD013 (MacroGenics). An example of a TIM-3 inhibitor is TSR-022 (GlaxoSmithKline plc).

In certain embodiments the checkpoint inhibitor is selected from nivolumab (OPDIVO®); pembrolizumab (KEYTRUDA®); and pidilizumab/CT-011, MPDL3280A/RG7446; MEDI4736; MSB0010718C; BMS 936559, a PDL2/lg fusion protein such as AMP 224 or an inhibitor of B7-H3 (e.g., MGA271), B7-H4, BTLA, HVEM, TIM3, GAL9, LAG 3, VISTA, KIR, 2B4, CD₁₆₀, CGEN-15049, CHK 1, CHK2, A2aR, B-7 family ligands, or a combination thereof.

In yet another embodiment, one or more of the active compounds described herein can be administered in an effective amount for the treatment of abnormal tissue of the female reproductive system such as breast, ovarian, endometrial, or uterine cancer, in combination or alternation with an effective amount of an estrogen inhibitor including, but not limited to, a SERM (selective estrogen receptor modulator), a SERD (selective estrogen receptor degrader), a complete estrogen receptor degrader, or another form of partial or complete estrogen antagonist or agonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors.

Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703,810, as well as US 2015/0005286, WO 2014/205136, and WO 2014/205138.

Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone.

Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684.

In another embodiment, active compounds described herein can be administered in an effective amount for the treatment of abnormal tissue of the male reproductive system such as prostate or testicular cancer, in combination or alternation with an effective amount of an androgen (such as testosterone) inhibitor including, but not limited to a selective androgen receptor modulator, a selective androgen receptor degrader, a complete androgen receptor degrader, or another form of partial or complete androgen antagonist. In certain embodiments, the prostate or testicular cancer is androgen resistant.

Non-limiting examples of anti-androgen compounds are provided in WO 2011/156518 and U.S. Pat. Nos. 8,455,534 and 8,299,112. Additional non-limiting examples of anti-androgen compounds include enzalutamide, apalutamide, cyproterone acetate, chlormadinone acetate, spironolactone, canrenone, drospirenone, ketoconazole, topilutamide, abiraterone acetate, and cimetidine.

In certain embodiments, the bioactive agent is an ALK inhibitor. Examples of ALK inhibitors include but are not limited to crizotinib (XALKORI©), alectinib (ALECENSA®), ceritinib, TAE684 (NVP-TAE684), GSK1838705A, AZD3463, ASP3026, PF-06463922, entrectinib (RXDX-101), and AP26113.

In certain embodiments, the bioactive agent is an HER-2 inhibitor. Examples of HER-2 inhibitors include trastuzumab, lapatinib, ado-trastuzumab emtansine, and pertuzumab.

In certain embodiments, the bioactive agent is a CD20 inhibitor. Examples of CD20 inhibitors include obinutuzumab (GAZYVA®), rituximab (RITUXAN®), ofatumumab, ibritumomab, tositumomab, and ocrelizumab.

In certain embodiments, the bioactive agent is a JAK3 inhibitor. Examples of JAK3 inhibitors include tasocitinib.

In certain embodiments, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide) (navitoclax), ABT-263 ((R)-4-(4-((4′-chloro-4,4-dimethyl-3,4,5,6-tetrahydro-[1, 1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-dimethylethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (oblimersen).

In certain embodiments, the bioactive agent is a kinase inhibitor. In certain embodiments, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include, but are not limited to, Wortmannin, demethoxyviridin, perifosine, idelalisib, pictilisib, palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((+)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4.5 h]isochromen-10-yl]acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422).

Examples of BTK inhibitors include ibrutinib (also known as PCI-32765) (IMBRUVICA®) (1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R-N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-f{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, but are not limited to, cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (GLEEVEC®; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In certain embodiments, the bioactive agent is a c-MET inhibitor, for example, crizotinib (XALKORI©, CRIZONIX™), tepotinib (XL880, EXEL-2880, GSK1363089, GSK089), or tivantinib (ARQ197).

In certain embodiments, the bioactive agent is an AKT inhibitor, including, but not limited to, MK-2206, GSK690693, perifosine, (KRX-0401), GDC-0068, triciribine, AZD5363, honokiol, PF-04691502, and miltefosine, a FLT-3 inhibitor, including, but not limited to, P406, dovitinib, quizartinib (AC220), amuvatinib (MP-470), tandutinib (MLN518), ENMD-2076, and KW-2449, or a combination thereof.

In certain embodiments, the bioactive agent is an mTOR inhibitor. Examples of mTOR inhibitors include, but are not limited to, rapamycin and its analogs, everolimus (AFINITOR®), temsirolimus, ridaforolimus, sirolimus, and deforolimus.

In certain embodiments, the bioactive agent is a RAS inhibitor. Examples of RAS inhibitors include but are not limited to Reolysin and siG12D LODER.

In certain embodiments, the bioactive agent is a HSP inhibitor. HSP inhibitors include but are not limited to geldanamycin or 17-N-allylamino-17-demethoxygeldanamycin (17AAG), and radicicol.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhibitor, a c-MET inhibitor, a PARP inhibitor, a Cdk inhibitor, an IGFR-TK inhibitor, an anti-HGF antibody, a focal adhesion kinase inhibitor, a Map kinase (MEK) inhibitor, a VEGF trap antibody, pemetrexed, panitumumab, amrubicin, oregovomab, Lep-etu, nolatrexed, azd2171, batabulin, of atumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, 1L13-PE38QQR, INO 1001, IPdRi KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, GLEEVEC®, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In certain embodiments the compound is administered in combination with ifosfamide.

In certain embodiments, the bioactive agent is selected from, but are not limited to, imatinib mesylate (GLEEVEC®), dasatinib (SPRYCEL®), nilotinib (TASIGNA®), bosutinib (BOSULIF®), trastuzumab (HERCEPTIN®), trastuzumab-DM1, pertuzumab (PERJETA®), lapatinib (TYKERB®), gefitinib (IRESSA®), erlotinib (TARCEVA®), cetuximab (ERBITUX®), panitumumab (VECTIBIX®), vandetanib (CAPRELSA®), vemurafenib (ZELBORAF®), vorinostat (ZOLINZA®), romidepsin (ISTODAX®), bexarotene (TAGRETIN®), alitretinoin (PANRETIN®), tretinoin (VESANOID®), carfilizomib (KYPROLIS®), pralatrexate (FOLOTYN®), bevacizumab (AVASTIN®), ziv-aflibercept (ZALTRAP®), sorafenib (NEXAVAR®), sunitinib (SUTENT®), pazopanib (VOTRIENT®), regorafenib (STIVARGA®), and cabozantinib (COMETRIQ®).

In certain aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: vincristine (ONCOVINE®) or liposomal vincristine (MARQIBO®), daunorubicin (daunomycin or CERUBIDINE®) or doxorubicin (ADRIAMYCIN®), cytarabine (cytosine arabinoside, ara-C, or CYTOSAR®), L-asparaginase (ELSPAR®) or PEG-L-asparaginase (pegaspargase or ONCASPAR®), etoposide (VP-16), teniposide (VUMON®), 6-mercaptopurine (6-MP or PURINETHOL®), methotrexate, cyclophosphamide (CYTOXAN®), prednisone, dexamethasone (DECADRON®), imatinib (GLEEVEC®), dasatinib (SPRYCEL®), nilotinib (TASIGNA®), bosutinib (BOSULIF®), and ponatinib (ICLUSIG®).

Examples of additional suitable chemotherapeutic agents include, but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), chlorambucil, cisplatin, cladribine, colchicin, conjugated estrogens, cyclophosphamide, cyclothosphamide, cytarabine, cytochalasin B, cytoxan, dacarbazine, dactinomycin, dactinomycin (formerly actinomycin), daunirubicin HCL, daunorucbicin citrate, denileukin diftitox, dexrazoxane, dibromomannitol, dihydroxy anthracin dione, docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

In some embodiments, the compound of the present invention is administered in combination with a chemotherapeutic agent (e.g., a cytotoxic agent or other chemical compound useful in the treatment of cancer). Examples of chemotherapeutic agents include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Inti. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the compound of the present invention. Suitable dosing regimens of combination chemotherapies are known in the ar. For example, combination dosing regimes are described in Saltz et al., Proc. Am. Soc. Clin. Oncol. 18:233a (1999) and Douillard et al., Lancet 355(9209): 1041-1047 (2000).

Additional therapeutic agents that can be administered in combination with a compound disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (ELOXATIN®), PD0332991, ribociclib (LEE011), amebaciclib (LY2835219), HDM201, fulvestrant (FASLODEX®), exemestane (AROMASIN®), PIM447, ruxolitinib (INC424), BGJ398, necitumumab, pemetrexed (ALIMTA®), and ramucirumab (IMC-1121B).

In certain embodiments, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. MAbs that bind to cell surface growth factor receptors prevent the targeted receptors from sending their normal growth-promoting signals. They may also trigger apoptosis and activate the immune system to destroy tumor cells.

In one aspect of the present invention, the bioactive agent is an immunosuppressive agent. The immunosuppressive agent can be a calcineurin inhibitor, e.g., a cyclosporin or an ascomycin, e.g., cyclosporin A (NEORAL*), FK506 (tacrolimus), pimecrolimus, a mTOR inhibitor, e.g., rapamycin or a derivative thereof, e.g., sirolimus (RAPAMUNE®), everolimus (CERTICAN®), temsirolimus, zotarolimus, biolimus-7, biolimus-9, a rapalog, e.g., ridaforolimus, azathioprine, campath 1H, a SiP receptor modulator, e.g., fingolimod or an analogue thereof, an anti IL-8 antibody, mycophenolic acid or a salt thereof, e.g., sodium salt, or a prodrug thereof, e.g., mycophenolate mofetil (CELLCEPT®), OKT3 (ORTHOCLONE OKT3®), prednisone, ATGAM®, THYMOGLOBULIN®, brequinar sodium, OKT4, T10B9.A-3A, 33B3.1, 15-deoxyspergualin, tresperimus, leflunomide (ARAVA®), CTLAI-Ig, anti-CD25, anti-IL2R, basiliximab (SIMULECT®), daclizumab (ZENAPAX®), mizorbine, methotrexate, dexamethasone, ISAtx-247, SDZ ASM 981 (pimecrolimus, ELIDEL®), CTLA41g (abatacept), belatacept, LFA31g,, etanercept (sold as ENBREL® by Immunex), adalimumab (HUMIRA®), infliximab (REMICADE®), an anti-LFA-1 antibody, natalizumab (ANTEGREN®), enlimomab, gavilimomab, antithymocyte immunoglobulin, siplizumab, alefacept, efalizumab, pentasa, mesalazine, asacol, codeine phosphate, benorylate, fenbufen, naprosyn, diclofenac, etodolac and indomethacin, aspirin and ibuprofen.

In some embodiments, the bioactive agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-1-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The combination therapy may include a therapeutic agent which is a non-drug treatment. For example, the compound could be administered in addition to radiation therapy, cryotherapy, hyperthermia, and/or surgical excision of tumor tissue.

### Additional Embodiments of the Present Invention

All separate embodiments may be combined unless excluded by context.

### Embodiments of R¹

In certain embodiments R¹ is hydrogen.

In certain embodiments R¹ is alkyl.

In certain embodiments R¹ is cycloalkyl.

In certain embodiments R¹ is methyl.

In certain embodiments R¹ is ethyl.

In certain embodiments R¹ is cyclopropyl.

### Embodiments of R²

In certain embodiments R² is hydrogen.

In certain embodiments R² is alkyl.

In certain embodiments R² is cycloalkyl.

In certain embodiments R² is methyl.

In certain embodiments R² is ethyl.

In certain embodiments R² is cyclopropyl.

In certain embodiments R² is haloalkyl.

In certain embodiments R² is CF₃.

In certain embodiments R¹ and R² together with the nitrogen atom to which they are attached form heterocycloalkyl optionally substituted with one or two R³.

### Embodiments of R^{2′}

In certain embodiments R^{2′} is hydrogen.

In certain embodiments R^{2′} is alkyl.

In certain embodiments R^{2′} is cycloalkyl.

In certain embodiments R^{2′} is methyl.

In certain embodiments R^{2′} is ethyl.

In certain embodiments R^{2′} is cyclopropyl.

In certain embodiments R^{2′} is haloalkyl.

In certain embodiments R^{2′} is CF₃.

In certain embodiments R¹ and R^{2′} together with the carbon atom to which they are attached form cycloalkyl optionally substituted with one or two R³.

### Embodiments of R³

In certain embodiments R³ is hydrogen.

In certain embodiments R³ is alkyl.

In certain embodiments R³ is cycloalkyl.

In certain embodiments R³ is methyl.

In certain embodiments R³ is ethyl.

In certain embodiments R³ is cyclopropyl.

In certain embodiments R³ is alkoxy.

In certain embodiments R³ is methoxy.

In certain embodiments R³ is ethoxy.

In certain embodiments R³ is halogen.

In certain embodiments R³ is F.

### Embodiments of R⁴

In certain embodiments R⁴ is hydrogen.

In certain embodiments R⁴ is alkyl.

In certain embodiments R⁴ is cycloalkyl.

In certain embodiments R⁴ is methyl.

In certain embodiments R⁴ is ethyl.

In certain embodiments R⁴ is cyclopropyl.

In certain embodiments R⁴ is halogen.

In certain embodiments R⁴ is F.

In certain embodiments R⁴ is cyano.

### Embodiments of R⁵

In certain embodiments R⁵ is hydrogen.

In certain embodiments R⁵ is alkyl.

In certain embodiments R⁵ is cycloalkyl.

In certain embodiments R⁵ is methyl.

In certain embodiments R⁵ is ethyl.

In certain embodiments R⁵ is cyclopropyl.

In certain embodiments R⁵ is halogen.

In certain embodiments R⁵ is F.

In certain embodiments R⁵ is cyano.

### Embodiments of R⁶

In certain embodiments R⁶ is hydrogen.

In certain embodiments R⁶ is alkyl.

In certain embodiments R⁶ is cycloalkyl.

In certain embodiments R⁶ is methyl.

In certain embodiments R⁶ is ethyl.

In certain embodiments R⁶ is cyclopropyl.

In certain embodiments R⁶ is halogen.

In certain embodiments R⁶ is F.

In certain embodiments R⁶ is hydroxy.

In certain embodiments R⁶ is amino.

In certain embodiments R⁶ is dialkylamino.

In certain embodiments R⁶ is alkoxy.

In certain embodiments R⁶ is alkoxyalkyl.

### Embodiments of R⁷

In certain embodiments R⁷ is hydrogen.

In certain embodiments R⁷ is alkyl.

In certain embodiments R⁷ is methyl

In certain embodiments R⁷ is ethyl.

### Embodiments of R⁸

In certain embodiments R⁸ is hydrogen.

In certain embodiments R⁸ is alkyl.

In certain embodiments R⁸ is cyano.

In certain embodiments R⁸ is halogen.

In certain embodiments R⁸ is alkoxy.

In certain embodiments R⁸ is fluorine.

In certain embodiments R⁸ is methoxy.

In certain embodiments R⁸ is ethoxy.

In certain embodiments R⁸ is methyl

In certain embodiments R⁸ is ethyl.

### Embodiments of R⁹

In certain embodiments R⁹ is hydrogen.

In certain embodiments R⁹ is alkyl.

In certain embodiments R⁹ is cyano.

In certain embodiments R⁹ is halogen.

In certain embodiments R⁹ is alkoxy.

In certain embodiments R⁹ is fluorine.

In certain embodiments R⁹ is methoxy.

In certain embodiments R⁹ is ethoxy.

In certain embodiments R⁹ is methyl

In certain embodiments R⁹ is ethyl.

### Embodiments of R¹⁷

In certain embodiments R¹⁷ is hydrogen.

In certain embodiments R¹⁷ is alkyl.

In certain embodiments R¹⁷ is cyano.

In certain embodiments R¹⁷ is halogen.

In certain embodiments R¹⁷ is alkoxy.

In certain embodiments R¹⁷ is fluorine.

In certain embodiments R¹⁷ is methoxy.

In certain embodiments R¹⁷ is ethoxy.

In certain embodiments R¹⁷ is methyl

In certain embodiments R¹⁷ is ethyl.

In certain embodiments R¹⁷ is hydroxy.

In certain embodiments R¹⁷ is cycloalkyl.

In certain embodiments R¹⁷ is cyclopropyl.

### Embodiments of R⁸

In certain embodiments R¹⁸ is hydrogen.

In certain embodiments R¹⁸ is alkyl.

In certain embodiments R¹⁸ is cyano.

In certain embodiments R¹⁸ is halogen.

In certain embodiments R¹⁸ is alkoxy.

In certain embodiments R¹⁸ is fluorine.

In certain embodiments R¹⁸ is methoxy.

In certain embodiments R¹⁸ is ethoxy.

In certain embodiments R¹⁸ is methyl

In certain embodiments R¹⁸ is ethyl.

In certain embodiments R¹⁸ is hydroxy.

In certain embodiments R¹⁸ is cycloalkyl.

In certain embodiments R¹⁸ is cyclopropyl.

### Embodiments of R¹⁹

In certain embodiments R¹⁹ is hydrogen.

In certain embodiments R¹⁹ is alkyl.

In certain embodiments R¹⁹ is cyano.

In certain embodiments R¹⁹ is halogen.

In certain embodiments R¹⁹ is alkoxy.

In certain embodiments R¹⁹ is fluorine.

In certain embodiments R¹⁹ is methoxy.

In certain embodiments R¹⁹ is ethoxy.

In certain embodiments R¹⁹ is methyl

In certain embodiments R¹⁹ is ethyl.

In certain embodiments R¹⁹ is hydroxy.

In certain embodiments R¹⁹ is cycloalkyl.

In certain embodiments R¹⁹ is cyclopropyl.

### Embodiments of R⁶⁰

In certain embodiments R⁶⁰ is isopropyl.

In certain embodiments R⁶⁰ is pyrrolidine-1-yl substituted with F.

In certain embodiments R⁶⁰ is pyrrolidine-1-yl substituted with OMe.

In certain embodiments R⁶⁰ is cyclopropyl.

### Embodiments of A¹

In certain embodiments A¹ is NR₂.

In certain embodiments A¹ is —CHR^{2′}—.

In certain embodiments A¹ is NH.

In certain embodiments A¹ is NCH₃.

In certain embodiments A¹ is —CH₂—.

### Embodiments of A²

In certain embodiments A² is —O—.

In certain embodiments A² is —NH—.

In certain embodiments A² is —(C═O)—.

### Embodiments of A⁵, A⁶ and A¹⁵

In certain embodiments A⁵ is —CH—.

In certain embodiments A⁵ is —N—.

In certain embodiments A⁶ is —CH—.

In certain embodiments A⁶ is —N—.

In certain embodiments A¹⁵ is —O—.

In certain embodiments A¹⁵ is —N—.

In certain embodiments A¹⁵ is bond.

### Embodiments of q

In certain embodiments, q is 0.

In certain embodiments, q is 1.

### Embodiments of p

In certain embodiments, p is 0.

In certain embodiments, p is 1.

In certain embodiments, p is 2.

### Embodiments of r

In certain embodiments, r is 0.

In certain embodiments, r is 1.

In certain embodiments, r is 2.

### Embodiments of w

In certain embodiments, w is 1.

In certain embodiments, w is 2.

### Embodiments of \textcircled{D}

In certain embodiments, cycle D is optionally substituted with one or two substituents independently selected from halogen, alkyl and alkoxy.

In certain embodiments, cycle D is substituted with one halogen substituent.

In certain embodiments, cycle D is substituted with two halogen substituents.

In certain embodiments, cycle D is substituted with one alkyl.

In certain embodiments, cycle D is substituted with two alkyl substituents.

In certain embodiments, cycle D is substituted with one alkoxy.

In certain embodiments, cycle D is substituted with two alkoxy substituents.

In certain embodiments, cycle D is substituted with halogen and alkyl.

In certain embodiments, cycle D is substituted with halogen and alkoxy.

In certain embodiments, cycle D is substituted with alkyl and alkoxy.

In certain embodiments, cycle D is

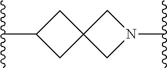 [SMILES: *C1CC2(C1)CN(*)C2]

(2-azaspiro[3.3]heptane-2,6-diyl).

In certain embodiments, cycle D is

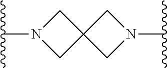 [SMILES: *N1CC2(C1)CN(*)C2]

(2,6-diazaspiro[3.3]heptane-2,6-diyl).

In certain embodiments, cycle D is

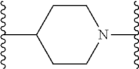 [SMILES: *C1CCN(*)CC1]

(piperidine-1,4-diyl).

### Embodiments of \textcircled{E}

In certain embodiments cycle E, is

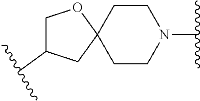 [SMILES: *C1COC2(CCN(*)CC2)C1]

(1-oxa-8-azaspiro[4.5]decane-3,8-diyl).

In certain embodiments cycle E, is

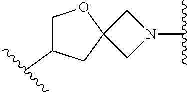 [SMILES: *C1COC2(C1)CN(*)C2]

(5-oxa-2-azaspiro[3.4]octane-2,7-diyl).

### Embodiments of \textcircled{F}

In certain embodiments, is cycle F is

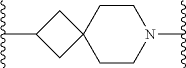 [SMILES: *C1CC2(CCN(*)CC2)C1]

(7-azaspiro[3.5]nonane-2,7-diyl).

In certain embodiments, is cycle F is

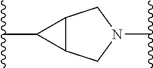 [SMILES: *C1C2CN(*)CC12]

(3-azabicyclo[3.1.0]hexane-3,6-diyl).

In certain embodiments, is cycle F is

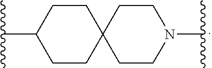 [SMILES: *C1CCC2(CC1)CCN(*)CC2]

(3-azaspiro[5.5]undecane-3,9-diyl).

In certain embodiments, is cycle F is

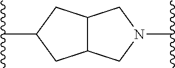 [SMILES: *C1CC2CN(*)CC2C1]

(octahydrocyclopenta[c]pyrrole-2,5-diyl).

In certain embodiments, is cycle F is

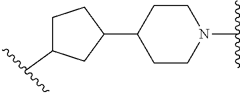 [SMILES: *C1CCC(C2CCN(*)CC2)C1]

(8-azaspiro[4.5]decane-2,8-diyl).

In certain embodiments, is cycle F is

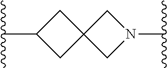 [SMILES: *C1CC2(C1)CN(*)C2]

(2-azaspiro[3.3]heptane-2,6-diyl).

In certain embodiments, is cycle F is

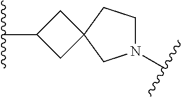 [SMILES: *C1CC2(CCN(*)C2)C1]

(6-azaspiro[3.4]octane-2,6-diyl).

### Embodiments of \textcircled{G}

In certain embodiments, cycle G is

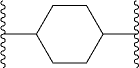 [SMILES: *C1CCC(*)CC1]

(cyclohexane-1,4-diyl).

In certain embodiments, cycle G is

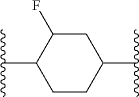 [SMILES: *C1CCC(*)C(F)C1]

(2-fluoro-cyclohexane-1,4-diyl).

In certain embodiments, cycle G is

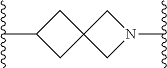 [SMILES: *C1CC2(C1)CN(*)C2]

(2-azaspiro[3.3]heptane-2,6-diyl).

In certain embodiments, cycle G is

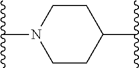 [SMILES: *C1CCN(*)CC1]

(piperidine-1,4-diyl).

In certain embodiments, cycle G is

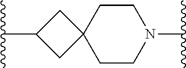 [SMILES: *C1CC2(CCN(*)CC2)C1]

(7-azaspiro[3.5]nonane-2,7-diyl).

In certain embodiments, cycle G is

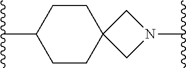 [SMILES: *C1CCC2(CC1)CN(*)C2]

(2-azaspiro[3.5]nonane-2,7-diyl).

In certain embodiments, cycle G is

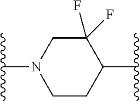 [SMILES: *C1CCN(*)CC1(F)F]

(3,3-difluoro-piperidine-1,4-diyl).

In certain embodiments, cycle G is

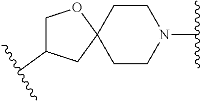 [SMILES: *C1COC2(CCN(*)CC2)C1]

(1-oxa-8-azaspiro[4.5]decane-3,8-diyl).

### Embodiments of \textcircled{H}

In certain embodiments, cycle H is

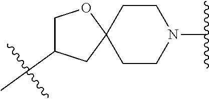 [SMILES: *C1COC2(CCN(*)CC2)C1]

(1-oxa-8-azaspiro[4.5]decane-3,8-diyl).

### Embodiments of \textcircled{I}

In certain embodiments, cycle I is

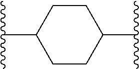 [SMILES: *C1CCC(*)CC1]

(cyclohexane-1,4-diyl).

### Embodiments of \textcircled{J}

In certain embodiments, cycle J is

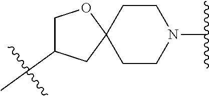 [SMILES: *C1COC2(CCN(*)CC2)C1]

(1-oxa-8-azaspiro[4.5]decane-3,8-diyl).

In certain embodiments, cycle J is

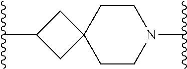 [SMILES: *C1CC2(CCN(*)CC2)C1]

(7-azaspiro[3.5]nonane-2,7-diyl).

In certain embodiments, cycle J is

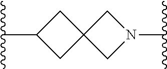 [SMILES: *C1CC2(C1)CN(*)C2]

(2-azaspiro[3.3]heptane-2,6-diyl).

### Embodiments of \textcircled{K}

In certain embodiments, cycle K is

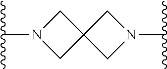 [SMILES: *N1CC2(C1)CN(*)C2]

(2,6-diazaspiro[3.3]heptane-2,6-diyl).

In certain embodiments, cycle K is

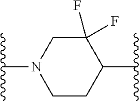 [SMILES: *C1CCN(*)CC1(F)F]

(3,3-difluoro-piperidine-1,4-diyl).

In certain embodiments, cycle K is

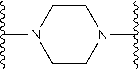 [SMILES: *N1CCN(*)CC1]

(piperazine-1,4-diyl).

### Embodiments of \textcircled{L}

In certain embodiments, cycle L is

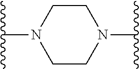 [SMILES: *N1CCN(*)CC1]

(piperazine-1,4-diyl).

In certain embodiments, cycle L is

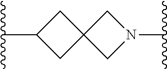 [SMILES: *C1CC2(C1)CN(*)C2]

(2-azaspiro[3.3]heptane-2,6-diyl).

In certain embodiments, cycle L is

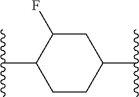 [SMILES: *C1CCC(*)C(F)C1]

(2-fluoro-cyclohexane-1,4-diyl).

In certain embodiments, cycle L is

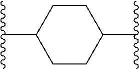 [SMILES: *C1CCC(*)CC1]

(cyclohexane-1,4-diyl).

In certain embodiments, cycle L is

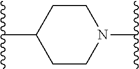 [SMILES: *C1CCN(*)CC1]

(piperidine-1,4-diyl).

### Embodiments of \textcircled{M}

In certain embodiments, cycle M is

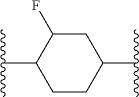 [SMILES: *C1CCC(*)C(F)C1]

2-fluoro-cyclohexane-1,4-diyl;

In certain embodiments, cycle M is

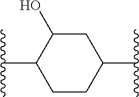 [SMILES: *C1CCC(*)C(O)C1]

2-hydroxy-cyclohexane-1,4-diyl.

### Embodiments of \textcircled{N}

In certain embodiments, cycle N is

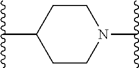 [SMILES: *C1CCN(*)CC1]

(piperidine-1,4-diyl).

In certain embodiments, cycle N is

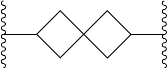 [SMILES: *C1CC2(C1)CC(*)C2]

(spiro[3.3]heptane-2,6-diyl).

In certain embodiments, cycle N is

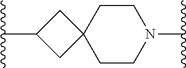 [SMILES: *C1CC2(CCN(*)CC2)C1]

(7-azaspiro[3.5]nonane-2,7-diyl).

In certain embodiments, cycle N is

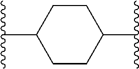 [SMILES: *C1CCC(*)CC1]

(cyclohexane-1,4-diyl).

In certain embodiments, cycle N is

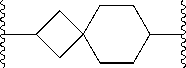 [SMILES: *C1CCC2(CC1)CC(*)C2]

(spiro[3.5]nonane-2,7-diyl).

In certain embodiments, cycle N is

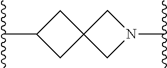 [SMILES: *C1CC2(C1)CN(*)C2]

(2-azaspiro[3.3]heptane-2,6-diyl).

### Embodiments of \textcircled{O}

In certain embodiments, cycle O is

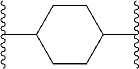 [SMILES: *C1CCC(*)CC1]

(cyclohexane-1,4-diyl).

In certain embodiments, cycle O is

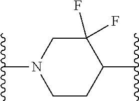 [SMILES: *C1CCN(*)CC1(F)F]

(3,3-difluoro-piperidine-1,4-diyl).

In certain embodiments, cycle O is

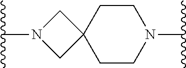 [SMILES: *N1CCC2(CC1)CN(*)C2]

(2,7-diazaspiro[3.5]nonane-2,7-diyl).

In certain embodiments, cycle O is

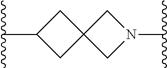 [SMILES: *C1CC2(C1)CN(*)C2]

(2-azaspiro[3.3]heptane-2,6-diyl).

In certain embodiments, cycle O is

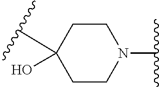 [SMILES: *N1CCC(*)(O)CC1]

(4-hydroxypiperidine-1,4-diyl).

In certain embodiments, cycle O is

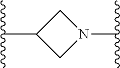 [SMILES: *C1CN(*)C1]

(azetidine-1,3-diyl).

In certain embodiments, cycle O is

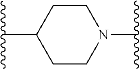 [SMILES: *C1CCN(*)CC1]

(piperidine-1,4-diyl).

### Embodiments of Alkyl

In certain embodiments, “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments, “alkyl” has one carbon.

In certain embodiments, “alkyl” has two carbons.

In certain embodiments, “alkyl” has three carbons.

In certain embodiments, “alkyl” has four carbons.

In certain embodiments, “alkyl” has five carbons.

In certain embodiments, “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of Cycloalkyl

In certain embodiments, “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In certain embodiments, “cycloalkyl” has three carbons.

In certain embodiments, “cycloalkyl” has four carbons.

In certain embodiments, “cycloalkyl” has five carbons.

In certain embodiments, “cycloalkyl” has six carbons.

In certain embodiments, “cycloalkyl” has seven carbons.

In certain embodiments, “cycloalkyl” has eight carbons.

In certain embodiments, “cycloalkyl” has nine carbons.

In certain embodiments, “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

### Embodiments of

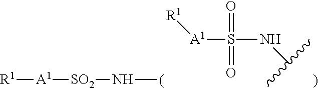

In certain embodiments,

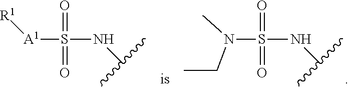

In certain embodiments,

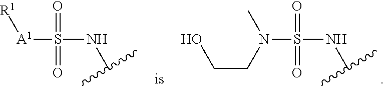

In certain embodiments,

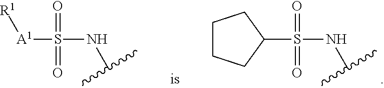

In certain embodiments,

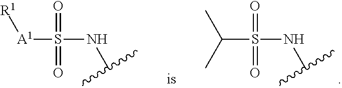

In certain embodiments,

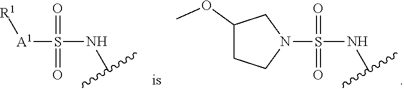

In certain embodiments,

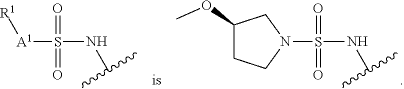

In certain embodiments,

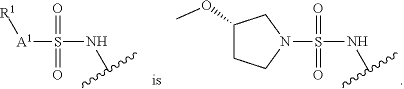

In certain embodiments,

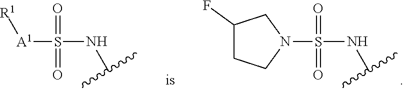

In certain embodiments,

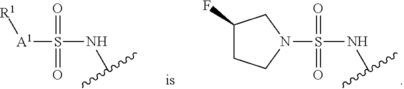

In certain embodiments,

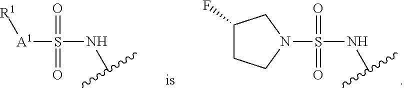

In certain embodiments,

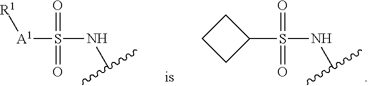

In certain embodiments,

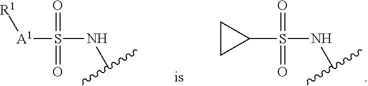

In certain embodiments,

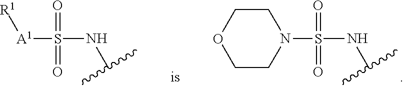

In certain embodiments,

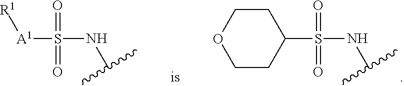

In certain embodiments,

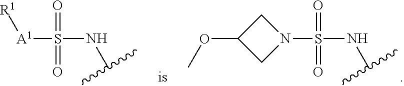

In certain embodiments,

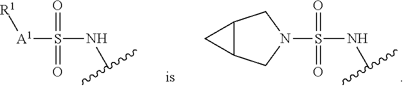

In certain embodiments,

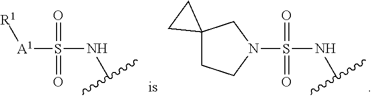

In certain embodiments,

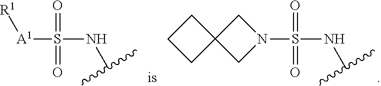

In certain embodiments,

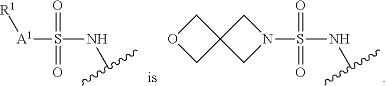

### Embodiments of Haloalkyl

In certain embodiments, “haloalkyl” is a C₁-C₁₀haloalkyl, C₁-C₉haloalkyl, C₁-C₈haloalkyl, C₁-C₇haloalkyl, C₁-C₆haloalkyl, C₁-C₄haloalkyl, C₁-C₄haloalkyl, C₁-C₃haloalkyl, and C₁-C₂haloalkyl.

In certain embodiments, “haloalkyl” has one carbon.

In certain embodiments, “haloalkyl” has one carbon and one halogen.

In certain embodiments, “haloalkyl” has one carbon and two halogens.

In certain embodiments, “haloalkyl” has one carbon and three halogens.

In certain embodiments, “haloalkyl” has two carbons.

In certain embodiments, “haloalkyl” has three carbons.

In certain embodiments, “haloalkyl” has four carbons.

In certain embodiments, “haloalkyl” has five carbons.

In certain embodiments, “haloalkyl” has six carbons.

Non-limiting examples of “haloalkyl” include:

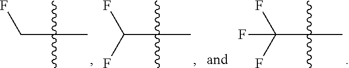

Additional non-limiting examples of “haloalkyl” include:

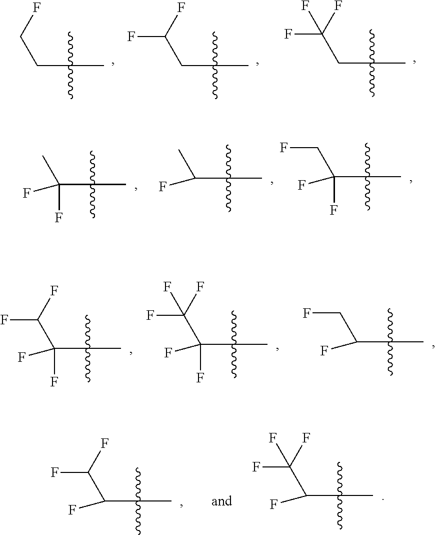

Additional non-limiting examples of “haloalkyl” include:

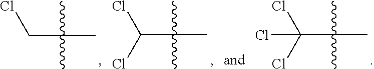

Additional non-limiting examples of “haloalkyl” include:

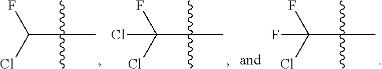

### Embodiments of Heterocycle

In certain embodiments, “heterocycle” refers to a cyclic ring with one nitrogen and one oxygen and 3, 4, 5, 6, 7, or 8 carbon atoms. In certain embodiments, “heterocycle” refers to a cyclic ring with one nitrogen and one oxygen and 4, 5, or 6 carbon atoms.

In certain embodiments, “heterocycle” refers to a cyclic ring with two nitrogens and 3, 4, 5, 6, 7, or 8 carbon atoms. In certain embodiments, “heterocycle” refers to a cyclic ring with two nitrogens and 4, 5, or 6 carbon atoms.

In certain embodiments, “heterocycle” refers to a cyclic ring with one oxygen and 3, 4, 5, 6, 7, or 8 carbon atoms. In certain embodiments, “heterocycle” refers to a cyclic ring with one oxygen and 3, 4, or 5 carbon atoms.

In certain embodiments, “heterocycle” refers to a cyclic ring with one sulfur and 3, 4, 5, 6, 7, or 8 carbon atoms. In certain embodiments, “heterocycle” refers to a cyclic ring with one sulfur and 3, 4, or 5 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocycle ring.

Non-limiting examples of “heterocycle” also include:

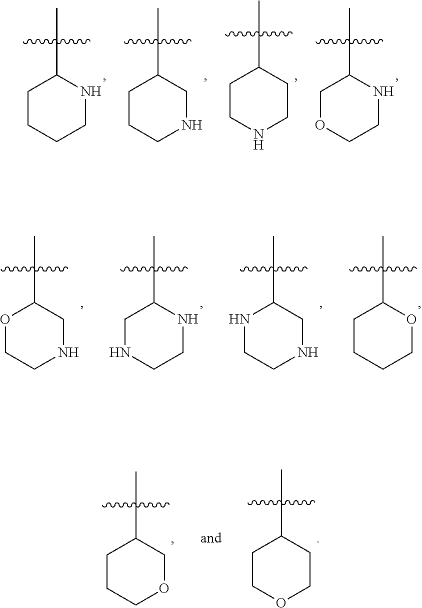

Additional non-limiting examples of “heterocycle” include

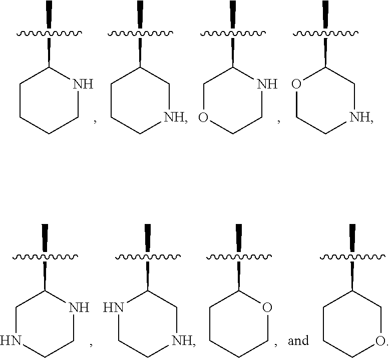

Additional non-limiting examples of “heterocycle” include:

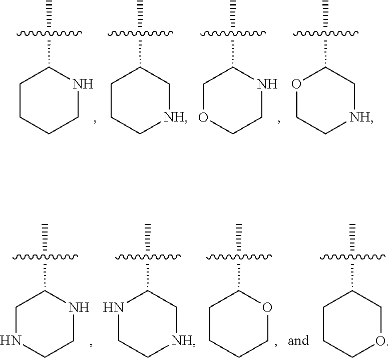

Non-limiting examples of “heterocycle” also include:

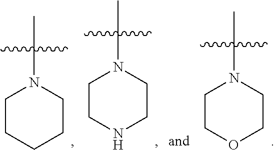

Non-limiting examples of “heterocycle” also include:

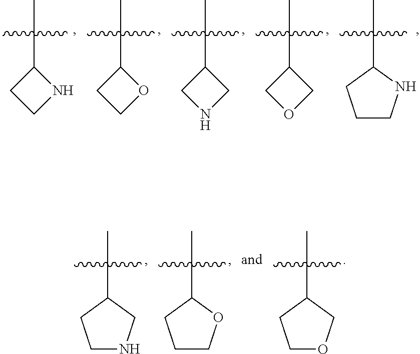

Additional non-limiting examples of “heterocycle” include:

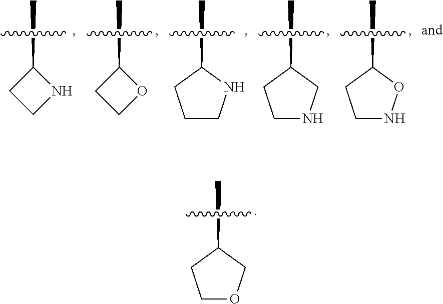

Additional non-limiting examples of “heterocycle” include:

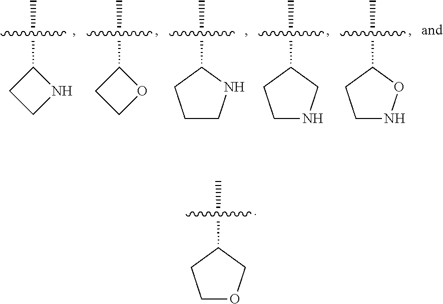

### Embodiments of Spiroheterocyclyl

In certain embodiments, “spiroheterocyclyl” refers to a heterocyclic moiety with one nitrogen atom and one oxygen atom and 5, 6, 7, 8, 9, or 10 carbon atoms having one quaternary carbon atom as the only common member of two rings.

In certain embodiments, “spiroheterocyclyl” refers to a heterocyclic moiety with two nitrogen atoms and 5, 6, 7, 8, 9, or 10 carbon atoms having one quaternary carbon atom as the only common member of two rings.

In certain embodiments, “spiroheterocyclyl” refers to a heterocyclic moiety with one oxygen atom and 5, 6, 7, 8, 9, or 10 carbon atoms having one quaternary carbon atom as the only common member of two rings.

In certain embodiments, “spiroheterocyclyl” is selected from:

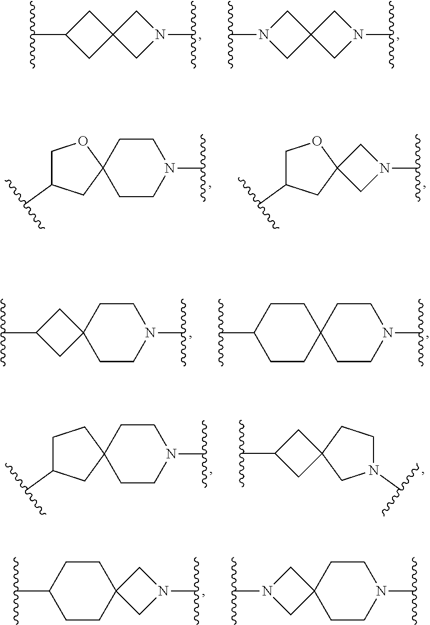

### Embodiments of Spirocycloalkyl

In certain embodiments, “spirocycloalkyl” refers to a cycloalkyl moiety with 6, 7, 8, 9, 10, or 11 carbon atoms having one quaternary carbon atom as the only common member of two rings.

In certain embodiments, “spirocycloalkyl” is

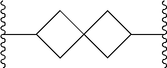 [SMILES: *C1CC2(C1)CC(*)C2]

(spiro[3.3]heptane-2,6-diyl).

In certain embodiments, “spirocycloalkyl” is

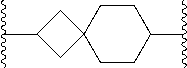 [SMILES: *C1CCC2(CC1)CC(*)C2]

(spiro[3.5]nonane-2,7-diyl).

### Embodiments of Fused Bicyclic Moiety (Fused Bicyclyl)

In certain embodiments, “fused” bicyclyl refers to a 6-, 7-, 8-, 9, 10-, 11-, or 12-membered bicyclyl moiety wherein two rings of the moiety share two adjacent atoms, that is, the rings (which may be heterocyclic or carbocyclic) share one covalent bond.

In certain embodiments, fused bicyclyl is a fused heterocyclyl with one or two heteroatoms selected from oxygen and nitrogen.

In certain embodiments, fused bicyclyl is a fused cycloalkyl.

In certain embodiments, fused bicyclyl contains one or two heteroatoms selected from oxygen and nitrogen and 5, 6, 7, 8, 9, or 10 carbon atoms.

In certain embodiments, fused bicyclyl contains one nitrogen atom and 5 carbon atoms.

In certain embodiments, fused bicyclyl contains one nitrogen atom and 6 carbon atoms.

In certain embodiments, fused bicyclyl contains one nitrogen atom and 7 carbon atoms.

In certain embodiments, fused bicyclic moiety is

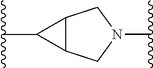 [SMILES: *C1C2CN(*)CC12]

(3-azabicyclo[3.1.0]hexane-3,6-diyl).

In certain embodiments, fused bicyclic moiety is

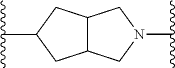 [SMILES: *C1CC2CN(*)CC2C1]

(octahydrocyclopenta[c]pyrrole-2,5-diyl).

### Embodiments of Bridged Bicyclic Moiety (Bridged Bicyclyl)

In certain embodiments, “bridged” bicyclyl refers to a 6-, 7-, 8-, 9-, 10-, or 11-membered bicyclyl moiety wherein two rings of the moiety share three or more atoms, separating the two bridgehead atoms by a bridge containing at least one atom.

In certain embodiments, bridged bicyclyl is a bridged heterocyclyl with one, two or three heteroatoms selected from oxygen and nitrogen.

In certain embodiments, bridged bicyclyl is a bridged heterocyclyl with one nitrogen atom, one oxygen atom and 7 carbon atoms.

In certain embodiments, bridged bicyclyl is a bridged heterocyclyl with two nitrogen atoms and 7 carbon atoms.

In certain embodiments, bridged bicyclyl is a bridged heterocyclyl with one oxygen atom and 8 carbon atoms.

In certain embodiments, bridged bicyclyl is a bridged heterocyclyl with one nitrogen atom and 8 carbon atoms.

In certain embodiments, bridged bicyclyl is a bridged cycloalkyl.

In certain embodiments, bridged bicyclyl is

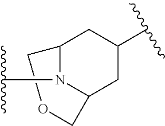 [SMILES: *C1CC2COCC(C1)N2*]

(3-oxa-9-azabicyclo[3.3.1]nonane-7,9-diyl).

In certain embodiments, bridged bicyclyl is

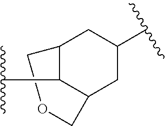 [SMILES: *C1CC2COCC(C1)C2*]

(3-oxabicyclo[3.3.1]nonane-7,9-diyl).

### Embodiments of BRAF Targeting Ligand Portion of the Molecule

In certain embodiments R¹ is CH₃.

In certain embodiments A¹ is —N(CH₂CH₃)—.

In certain embodiments R⁴ is cyano.

In certain embodiments R⁴ is F.

In certain embodiments R⁵ is F.

In certain embodiments, A² is O.

In certain embodiments, A² is NH.

In certain embodiments R⁶ is hydrogen.

In certain embodiments R⁶ is F.

In certain embodiments, the BRAF Targeting Ligand is selected from:

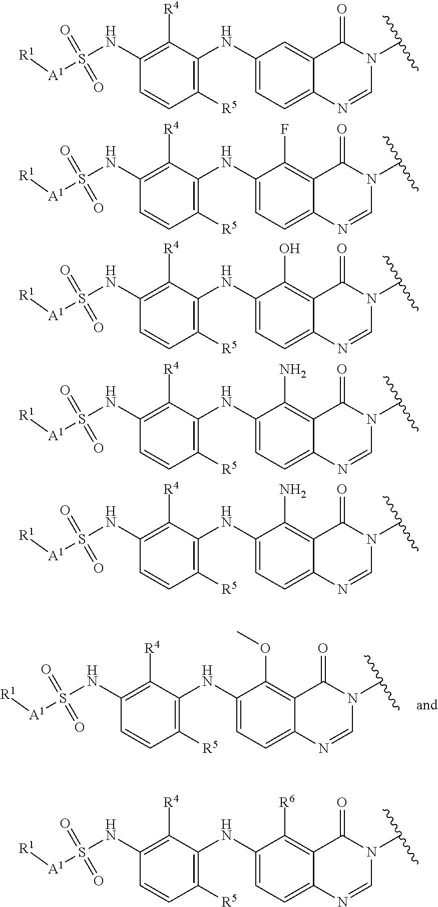

In certain embodiments, the BRAF Targeting Ligand is selected from:

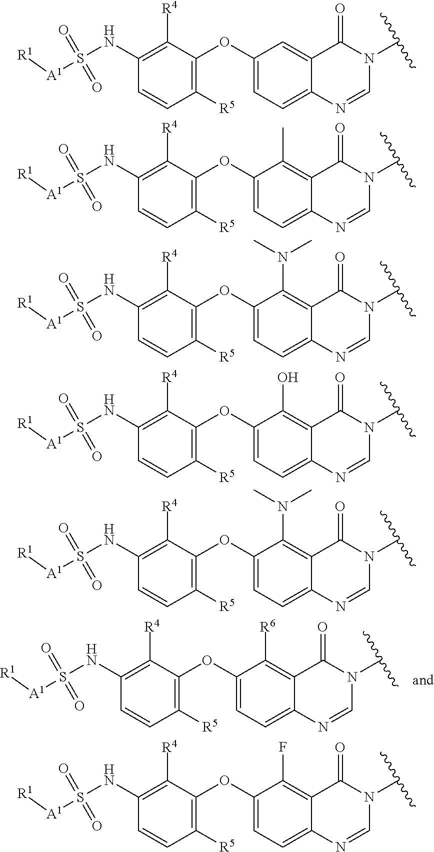

In certain embodiments, the BRAF Targeting Ligand is selected from:

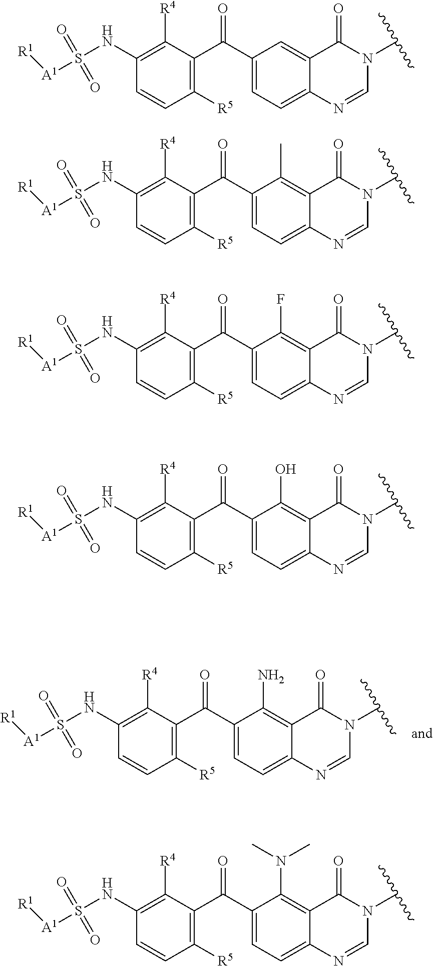

In certain embodiments, the BRAF Targeting Ligand is selected from:

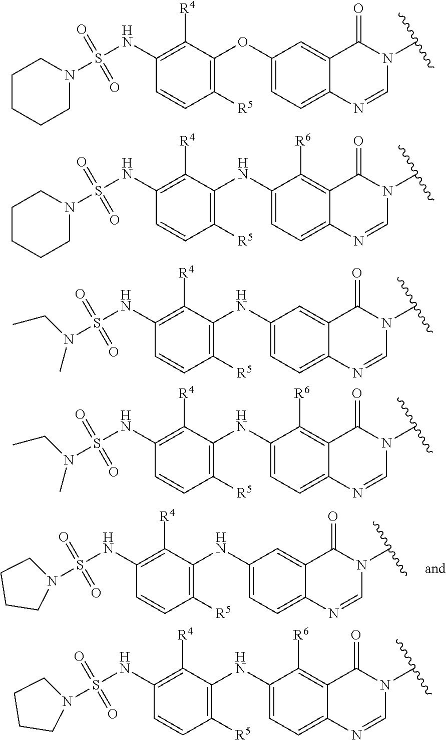

In certain embodiments, the BRAF Targeting Ligand is selected from:

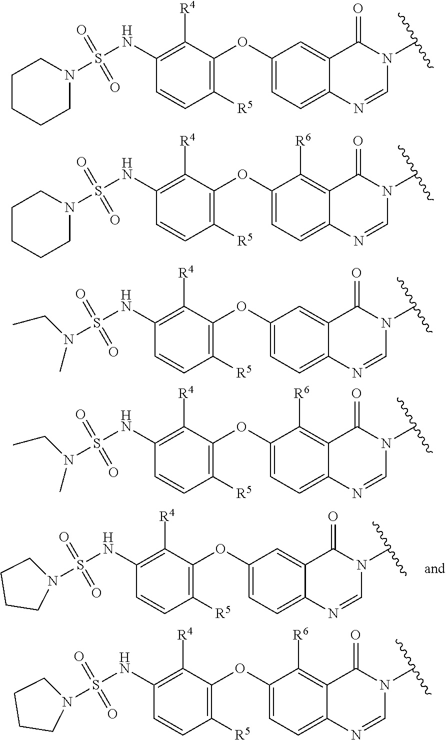

In certain embodiments, the BRAF Targeting Ligand is selected from:

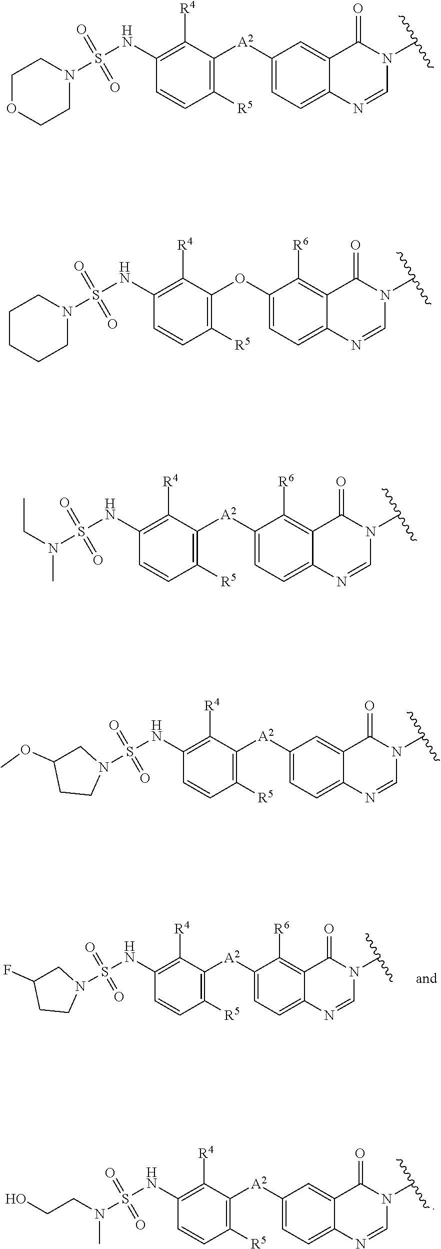

In certain embodiments, the BRAF Targeting Ligand is selected from:

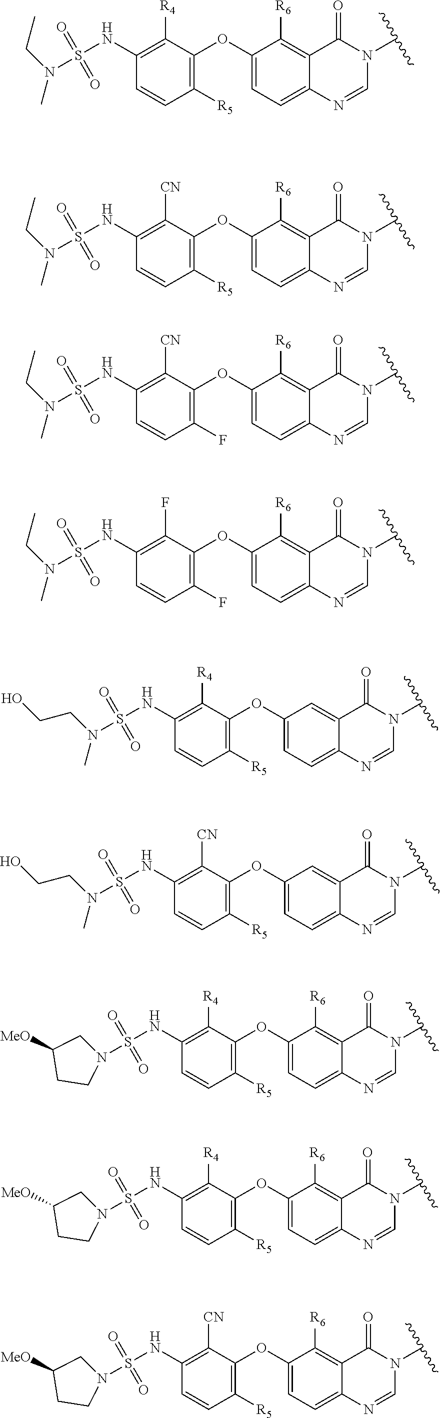

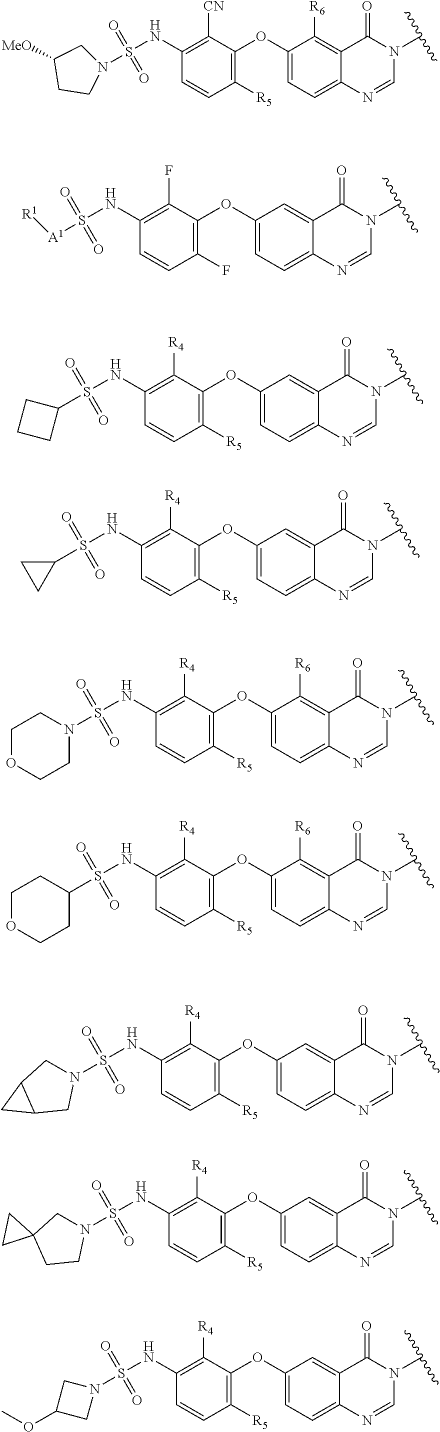

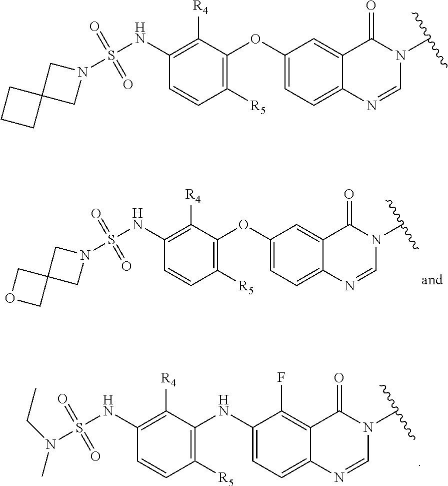

In certain embodiments, the BRAF Targeting Ligand is selected from:

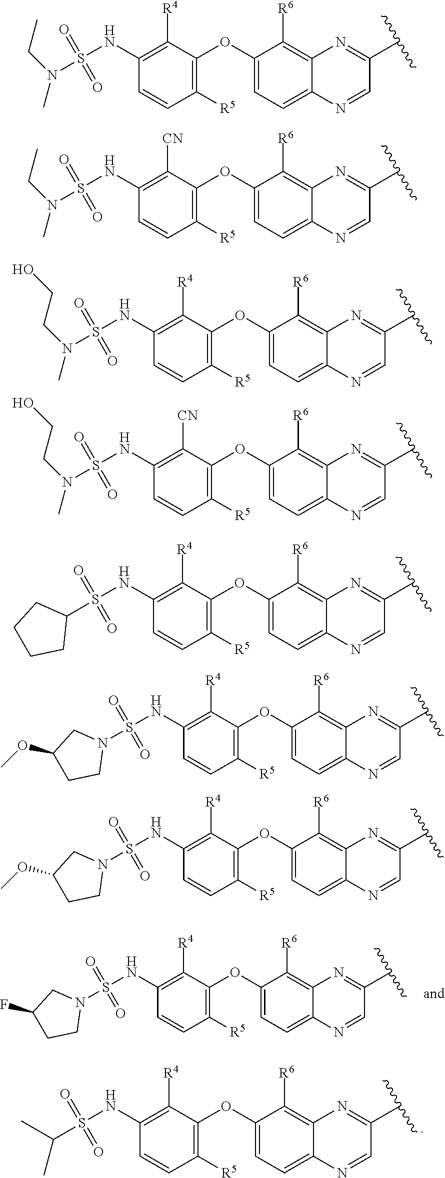

In certain embodiments, the BRAF Targeting Ligand is selected from:

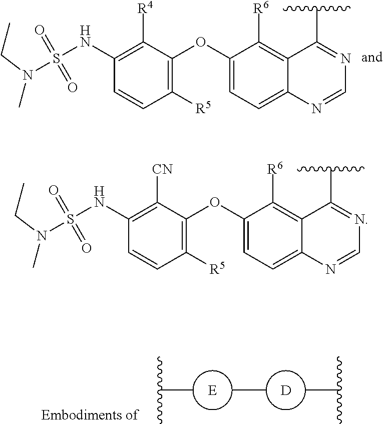

In certain embodiments,

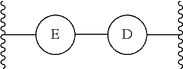

is selected from the group:

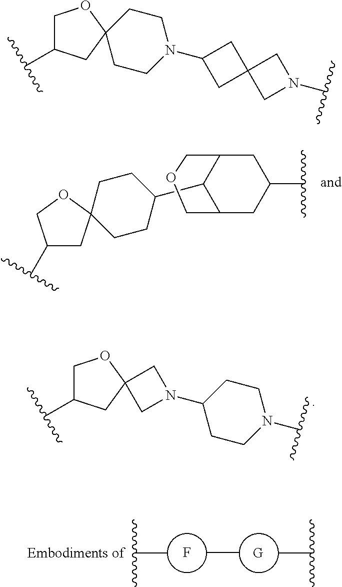

In certain embodiments,

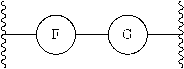

is selected from the group:

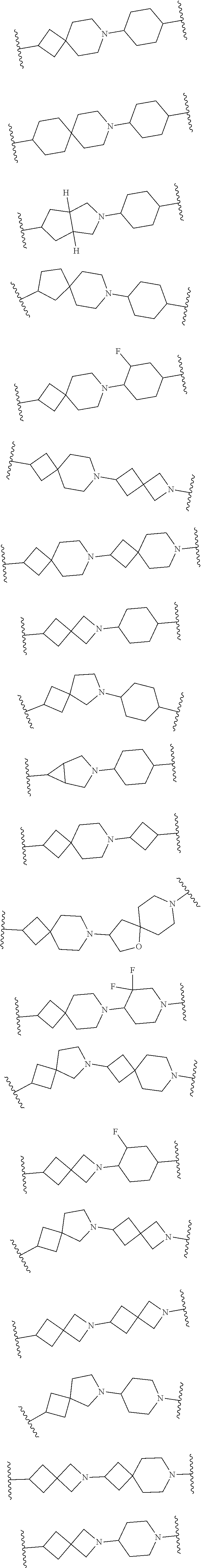

and stereoisomers thereof.

### Embodiments of Cereblon Ligand Portion of the Molecule

In certain embodiments, the Cereblon Ligand is selected from

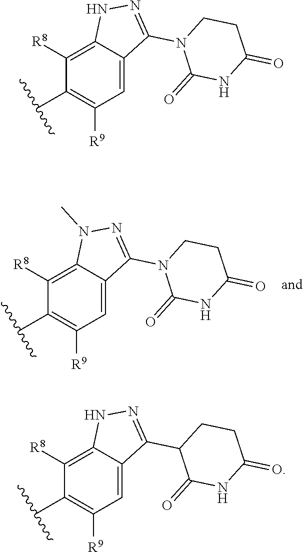

In certain embodiments, the Cereblon Ligand is selected from

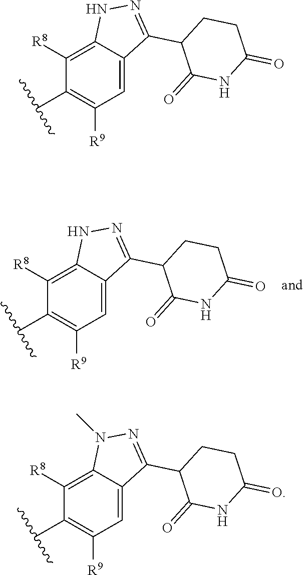

In certain embodiments, the Cereblon Ligand is selected from

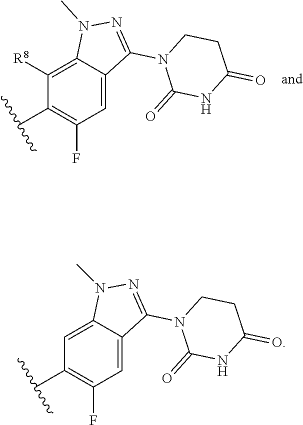

In certain embodiments, the Cereblon Ligand is selected from

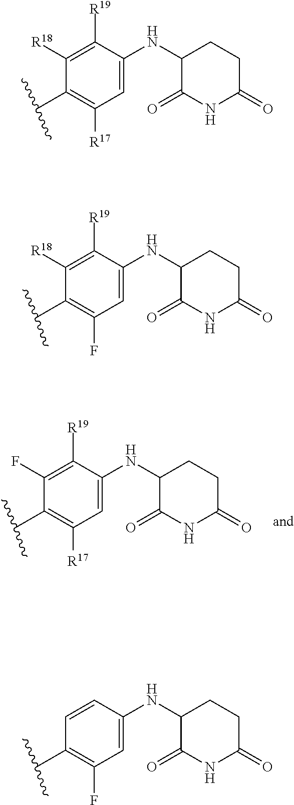

### Pharmaceutical Compositions

A selected compound of the present invention or its pharmaceutically acceptable salt can be administered as the neat chemical, but is often administered as a pharmaceutical composition, that includes an effective amount for a host, typically a human, in need of such treatment for any of the disorders described herein. Accordingly, the disclosure provides pharmaceutical compositions comprising an effective amount of compound or pharmaceutically acceptable salt together with at least one pharmaceutically acceptable carrier for any of the uses described herein. The pharmaceutical composition may contain a compound or salt as the only active agent, or, in an alternative embodiment, the compound and at least one additional active agent.

In certain embodiments the pharmaceutical composition is in a dosage form that contains from about 0.001 mg to about 1000 mg, from about 0.01 mg to about 800 mg, from about 1 mg to about 800 mg, or from about 200 mg to about 600 mg of the active compound and optionally from about 0.1 mg to about 1000 mg, from about 100 mg to about 800 mg, or from about 200 mg to about 600 mg of an additional active agent in a unit dosage form. Examples are dosage forms with at least about, or no more than, 0.001, 0.005, 0.010, 0.10, 1, 5, 10, 25, 50, 100, 200, 250, 300, 400, 500, 600, 700, or 750 mg of active compound, or its salt.

In certain aspects a compound of the present invention or a pharmaceutically acceptable salt thereof is administered at a dose between about 0.1 mg and about 1000 mg. In certain embodiments a compound of the present invention or a pharmaceutically acceptable salt thereof is administered at a dose between about 1 mg and about 500 mg. In certain embodiments a compound of the present invention or a pharmaceutically acceptable salt thereof is administered at a dose between about 10 mg and about 500 mg. In certain embodiments a compound of the present invention or a pharmaceutically acceptable salt thereof is administered at a dose between about 1 mg and about 250 mg.

In certain embodiments the pharmaceutical composition is in a dosage form that contains about 70 mg of active compound or its salt. In certain embodiments the pharmaceutical composition is in a dosage form that contains about 400 mg of active compound or its salt. In certain embodiments the pharmaceutical composition is in a dosage form that contains about 800 mg of active compound or its salt.

In certain embodiments the compound is administered twice per day to a patient in need thereof.

Compounds disclosed herein may be administered orally, topically, systemically, parenterally, by inhalation or spray, sublingually, via implant, including ocular implant, transdermally, via buccal administration, rectally, as an ophthalmic solution, injection, including intravenous, intra-aortal, intracranial, subdermal, intraperitoneal, subcutaneous, transnasal, sublingual, or rectal or by other means, in dosage unit formulations containing conventional pharmaceutically acceptable carriers.

The pharmaceutical composition may be formulated as any pharmaceutically useful form, e.g., as a solid dosage form, liquid, an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and should be of sufficiently high purity and sufficiently low toxicity to render them suitable for administration in an effective amount to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions/combinations can be formulated for oral administration. These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. % or from about 5 wt. % to about 75 wt. % of the compound.

In certain embodiments the LNP contains a cationic or ionizable limit. Examples include but are not limited to: U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are each herein incorporated by reference in their entirety for all purposes.

Formulations suitable for rectal administration are sometimes presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and sometimes take the form of an optionally buffered aqueous solution of the active compound. In certain embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

In certain aspects the term “a pharmaceutically acceptable salt” refers to a salt that is suitable for use in contact with the tissues of humans and animals. Examples of suitable salts with inorganic and organic acids include, but are not limited to acetic acid, citric acid, formic acid, fumaric acid, hydrochloric acid, lactic acid, maleic acid, malic acid, methane-sulfonic acid, nitric acid, phosphoric acid, p-toluene sulphonic acid, succinic acid, sulfuric acid (sulphuric acid), tartaric acid, trifluoroacetic acid and the like. Particular acids are formic acid, trifluoroacetic acid and hydrochloric acid.

In certain aspects the term “pharmaceutically acceptable auxiliary substance” refers to carriers and auxiliary substances such as diluents or excipients that are compatible with the other ingredients of the formulation.

In certain aspects the term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

In certain embodiments the term “pharmaceutically acceptable excipient” denotes an ingredient that has no therapeutic activity and is sufficiently non-toxic, such as a disintegrator, a binder, a filler, a solvent, a buffer, a tonicity agent, a stabilizer, an antioxidant, a surfactant or a lubricant used in formulating a pharmaceutical product.

### Additional Pharmaceutical Compositions

A compound of the present invention and/or the pharmaceutically acceptable salts thereof can be used as therapeutically active substances, e.g., in the form of pharmaceutical preparations. The pharmaceutical preparations can be administered orally, e.g., in the form of tablets, coated tablets, dragées, hard and soft gelatin capsules, solutions, emulsions or suspensions. The administration can, however, also be affected rectally, e.g., in the form of suppositories, or parenterally, e.g., in the form of injection solutions.

A compound of the present invention and/or the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

### General Synthesis

The compounds described herein can be prepared by methods known by those skilled in the art. In one non-limiting example, the disclosed compounds can be made using the schemes below.

Compounds of the present invention with stereocenters may be drawn without stereochemistry for convenience. One skilled in the art will recognize that pure enantiomers and diastereomers can be prepared by methods known in the art. Examples of methods to obtain optically active materials include at least the following
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the enantiomer is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step in the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved by chiral catalysts or chiral auxiliaries;
vi) diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer;
vii) first-and second-order asymmetric transformations—a technique whereby diastereomers from the racemate quickly equilibrate to yield a preponderance in solution of the diastereomer from the desired enantiomer of where preferential crystallization of the diastereomer from the desired enantiomer perturbs the equilibrium such that eventually in principle all the material is converted to the crystalline diastereomer from the desired enantiomers. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase (including vial chiral HPLC). The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is place in contact with a thin membrane barrier. The barrier may separate two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through;
xiv) simulated moving bed chromatography is used in one embodiment. A wide variety of chiral stationary phases are commercially available.

### Synthesis of Representative Compounds of the Present Invention

### Abbreviations

ACN=acetonitrile; Boc=tert-butyloxycarbonyl; dba=dibenzylideneacetone; COMU=(1-cyano-2-ethoxy-2-oxoethylidenaminooxy)dimethylamino-morpholino-carbenium hexafluorophosphate, 1-[(1-(cyano-2-ethoxy-2-oxoethylidenaminooxy)-dimethylamino-morpholino)]-uronium-hexafluorophosphate; DBU=1,8-diazabicyclo[5.4.0]undec-7-ene; DCM=dichloromethane; DMAc=dimethylacetamide; DMAP=4-dimethylaminopyridine; DMF=dimethylformamide; DMSO=dimethyl sulfoxide; dppf=1,1′-bis(diphenylphosphino)ferrocene; ESI=electrospray ionization; EtOAc=ethyl acetate; Ex=example; HATU=hexafluorophosphate azabenzotriazole tetramethyl uronium; IIPLC=high performance liquid chromatogaphy; IPA=isopropanol; LC-MS=liquid chromatography coupled with mass spectrometry; MS=mass spectrometry; MTBE=methyl tert-butyl ether; NBS=N-bromosuccinimide; NIS=N-iodosuccinimide; NMR=nuclear magnetic resonance; PEPPSI=pyridine-enhanced precatalyst preparation, stabilization, and initiation; PG=protecting group; pin=pinacolato; rt=room temperature; SFC=supercritical fluid chromatography; TEA=triethylamine; Tf=triflate; TFA=trifluoroacetic acid; THF=tetrahydrofuran; TLC=thin layer chromatography; Ts=tosylate; UPLC=ultra performance liquid chromatography.

### Example 1: Synthesis of Targeting Ligands

### Synthesis A1: Synthesis of (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1)

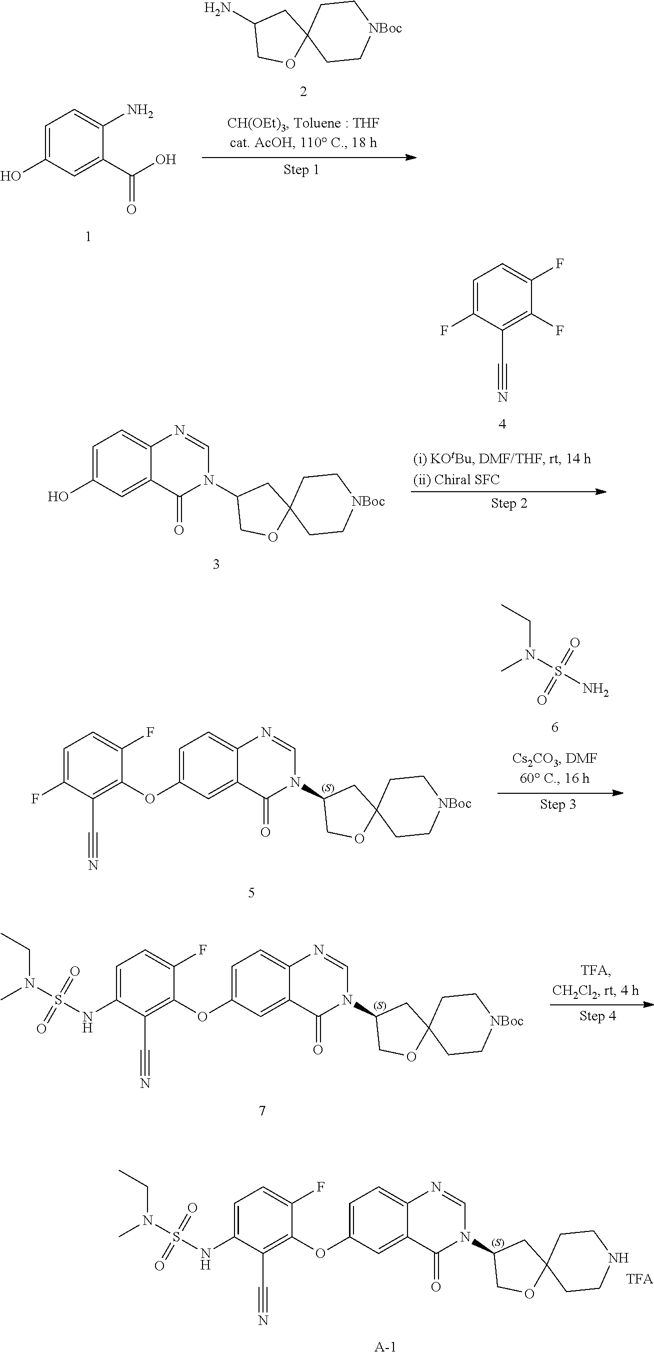

### Step 1

To a stirred solution of 2-amino-5-hydroxy-benzoic acid (1, 1.19 g, 7.80 mmol) in toluene: tetrahydrofuran (5:1) was added anhydrous triethyl orthoformate (9.36 mmol, 1.56 mL) at room temperature followed by addition of tert-butyl 3-amino-1-oxa-8-azaspiro[4.5]decane-8-carboxylate (2, 2 g, 7.80 mmol) and the resulting reaction mixture was heated at 110° C. for 18 hours in a sealed tube. After completion, the reaction mixture was cooled to room temperature. To the reaction mixture was added aqueous NaHCO₃ solution and the aqueous layers were extracted with ethyl acetate. The combined organic layers were washed with saturated NaCl solution, dried over anhydrous Na₂SO₄, filtered and filtrate was evaporated under reduced pressure to get the desired crude. The obtained crude product was further purified by silica gel chromatography using 5% methanol/dichloromethane as eluent to afford tert-butyl 3-(6-hydroxy-4-oxo-quinazolin-3-yl)-1-oxa-8-azaspiro[4.5]decane-8-carboxylate (3, 1.5 g, 3.09 mmol, 40% yield) as pale brown thick oil. LC-MS(ES+): m/z 402.3 [M+H]⁺.

### Step 2

To a stirred solution of tert-butyl 3-(6-hydroxy-4-oxo-quinazolin-3-yl)-1-oxa-8-azaspiro[4.5]decane-8-carboxylate (3, 1.8 g, 4.48 mmol) in N,N-dimethylformamide/tetrahydrofuran (10 mL) was added potassium 2-methylpropan-2-olate (603.75 mg, 5.38 mmol) and 2,3,6-trifluorobenzonitrile (4, 4.93 mmol, 569.70 μL) at room temperature. The reaction mixture was stirred at room temperature for 14 hours. After completion, water was added to the reaction mixture, which was then extracted with ethyl acetate. The combined organic layers were evaporated under reduced pressure to get the crude. Crude compound was purified by silica gel column chromatography eluted with 80-90% of ethyl acetate in petroleum ether to give racemic tert-butyl 3-[6-(2-cyano-3,6-difluoro-phenoxy)-4-oxo-quinazolin-3-yl]-1-oxa-8-azaspiro[4.5]decane-8-carboxylate. This racemic compound was subjected to chiral SFC method development followed by purification using Lux A1 chiral column (Solvent system-30% of (0.5% isopropyl amine in isopropyl alcohol, temperature −35° C.) to give tert-butyl(S)-3-(6-(2-cyano-3,6-difluorophenoxy)-4-oxoquinazolin-3(4H)-yl)-1-oxa-8-azaspiro[4.5]decane-8-carboxylate (5-S, first eluting isomer, 400 mg, 678.87 μmol, 15% yield) and tert-butyl (R)-3-(6-(2-cyano-3,6-difluorophenoxy)-4-oxoquinazolin-3(4H)-yl)-1-oxa-8-azaspiro[4.5]decane-8-carboxylate (5-R, second eluting isomer, 410 mg) as pale brown solid. LC-MS(ES+): m/z 483.1 [M+H-^{t}Bu]⁺.

### Step 3

To a solution of intermediate tert-butyl (3S)-3-[6-(2-cyano-3,6-difluoro-phenoxy)-4-oxo-quinazolin-3-yl]-1-oxa-8-azaspiro[4.5]decane-8-carboxylate (5-S, 410 mg, 761.32 μmol) in N,N-dimethylformamide was added Cs₂CO₃ (620.1 mg, 1.9 mmol) and [methyl(sulfamoyl)amino]ethane (6, 157.81 mg, 1.14 mmol) at room temperature. The resulting reaction mixture was stirred at 60° C. for 16 hours. After completion of the reaction, the mixture was cooled to room temperature, diluted with water and extracted with ethyl acetate. The combined organic layers were washed with saturated NaCl solution, dried over Na₂SO₄, filtered, and concentrated under reduced pressure to get the crude. The crude compound was purified by silica gel column chromatography with 20-50% ethyl acetate in petroleum ether as eluent to afford tert-butyl (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane-8-carboxylate (7, 471 mg, 670.36 μmol, 88% yield) as pale brown oil. LC-MS(ES−): m/z 655.21 [M−H]⁻.

### Step 4

A solution of tert-butyl (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane-8-carboxylate (7, 470 mg, 715.67 μmol) was taken in dichloromethane and added trifluoroacetic acid (3.58 mmol, 275.69 μL) at 0° C. The resulting reaction mixture was stirred at room temperature for 2 hours. After completion, reaction solvent was removed under reduced pressure to get crude product. Crude compound was triturated with methyl tert-butyl ether to get targeting ligand (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1, 436 mg, 494.10 μmol, 69% yield). LC-MS(ES+): m/z 557.1 [M+H]⁺.

### Synthesis A2: Synthesis of N-[2-cyano-4-fluoro-3-[3-[(3R)-1-oxa-8-azaspiro[4.5]decan-3-yl]-4-oxo-quinazolin-6-yl]oxy-phenyl]cyclopropanesulfonamide (A-2)

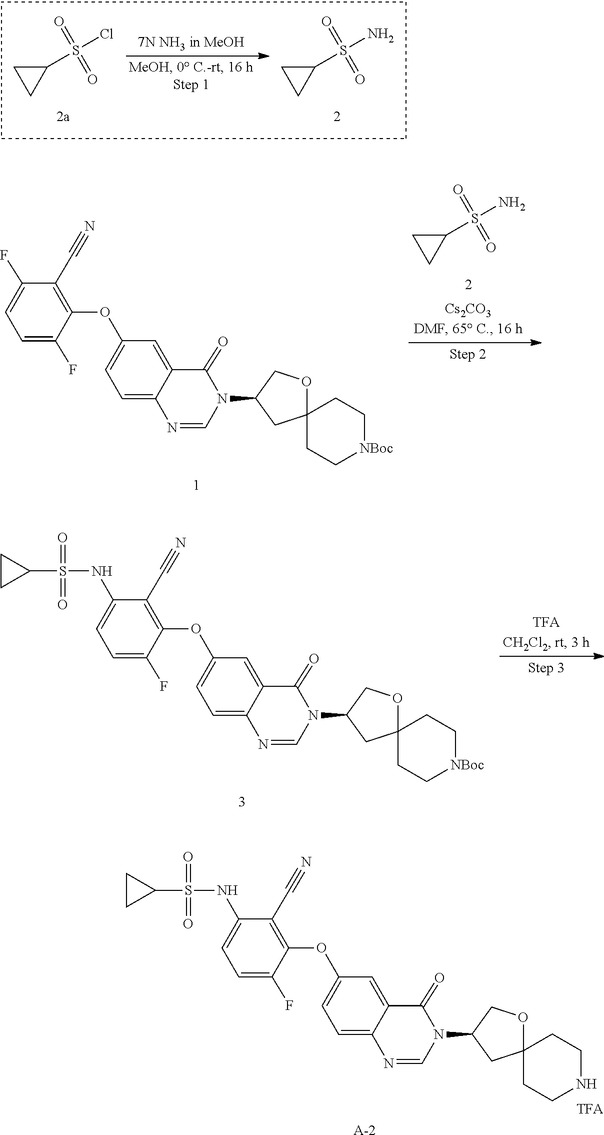

### Step 1

Into a 100 mL two neck round bottom flask containing a well stirred solution of cyclopropanesulfonyl chloride (2a, 2 g, 14.23 mmol, 1.45 mL) in methanol (30 mL) was added ammonia in 7N methanol (7 M, 40 mL) at 0° C. The reaction was stirred at room temperature for 16 hours. After completion of the reaction, solvent was concentrated under reduced pressure to get a crude residue. The residue was diluted with water (10 mL), solid precipitate was observed. The precipitated solid compound was filtered (400 mg). The filtrate was extracted with ethyl acetate (2×30 mL). The organic layer was dried over anhydrous Na₂SO₄, filtered and concentrated under reduced pressure to afford cyclopropanesulfonamide (2, 1.6 g, 11.89 mmol, 84% yield) as an off-white solid. ¹H NMR (400 MHz, DMSO-d₆): δ 6.78 (s, 2H), 2.55-2.48 (m, 1H), 0.92-0.88 (m, 4H).

### Step 2

Into a 20 mL reaction vial containing a solution of tert-butyl(3R)-3-[6-(2-cyano-3,6-difluoro-phenoxy)-4-oxo-quinazolin-3-yl]-1-oxa-8-azaspiro[4.5]decane-8-carboxylate (1, 500 mg, 928.43 mol) in N,N-dimethylformamide (6 mL) were added Cs₂CO₃ (605.00 mg, 1.86 mmol) and cyclopropanesulfonamide (2, 134.98 mg, 1.11 mmol) at room temperature. The reaction was stirred at 65° C. for 16 hours. After completion of the reaction, the reaction mixture was cooled to room temperature, quenched with water (30 mL) and extracted with ethyl acetate (2×40 mL). The organic layer was dried over anhydrous Na₂SO₄, filtered and the filtrate was concentrated under reduced pressure to get a crude residue which was purified by flash column chromatography (silica gel 230-400 mesh) eluted with 0-100% ethyl acetate/petroleum ether, where compound eluted at 70-80% ethyl acetate/petroleum ether to afford tert-butyl(3R)-3-[6-[2-cyano-3-(cyclopropylsulfonylamino)-6-fluoro-phenoxy]-4-oxo-quinazolin-3-yl]-1-oxa-8-azaspiro[4.5]decane-8-carboxylate (3, 500 mg, 539.32 mol, 58% yield) as a brown gummy liquid. UPLC-MS (ES+): m/z 584.0 [M-Boc+H]⁺.

### Step 3

Into a 50 mL single neck round bottom flask containing a well stirred solution of tert-butyl(3R)-3-[6-[2-cyano-3-(cyclopropylsulfonylamino)-6-fluoro-phenoxy]-4-oxo-quinazolin-3-yl]-1-oxa-8-azaspiro[4.5]decane-8-carboxylate (3, 500 mg, 781.62 mol) in dichloromethane (10 mL) was added trifluoroacetic acid (7.82 mmol, 598.13 L) at 0° C. The reaction was stirred at room temperature for 3 hours. After completion of the reaction, solvent was concentrated to afford N-[2-cyano-4-fluoro-3-[3-[(3R)-1-oxa-8-azaspiro[4.5]decan-3-yl]-4-oxo-quinazolin-6-yl]oxy-phenyl]cyclopropanesulfonamide (A-2, 500 mg, 734.39 mol, 94% yield, TFA salt) as a brown gummy liquid. UPLC-MS (ES+): m/z 540.0 [M+H]⁺.

### Synthesis A3: Synthesis of N-[2-cyano-4-fluoro-3-[3-[(3R)-1-oxa-8-azaspiro[4.5]decan-3-yl]-4-oxo-quinazolin-6-yl]oxy-phenyl]morpholine-4-sulfonamide (A-3)

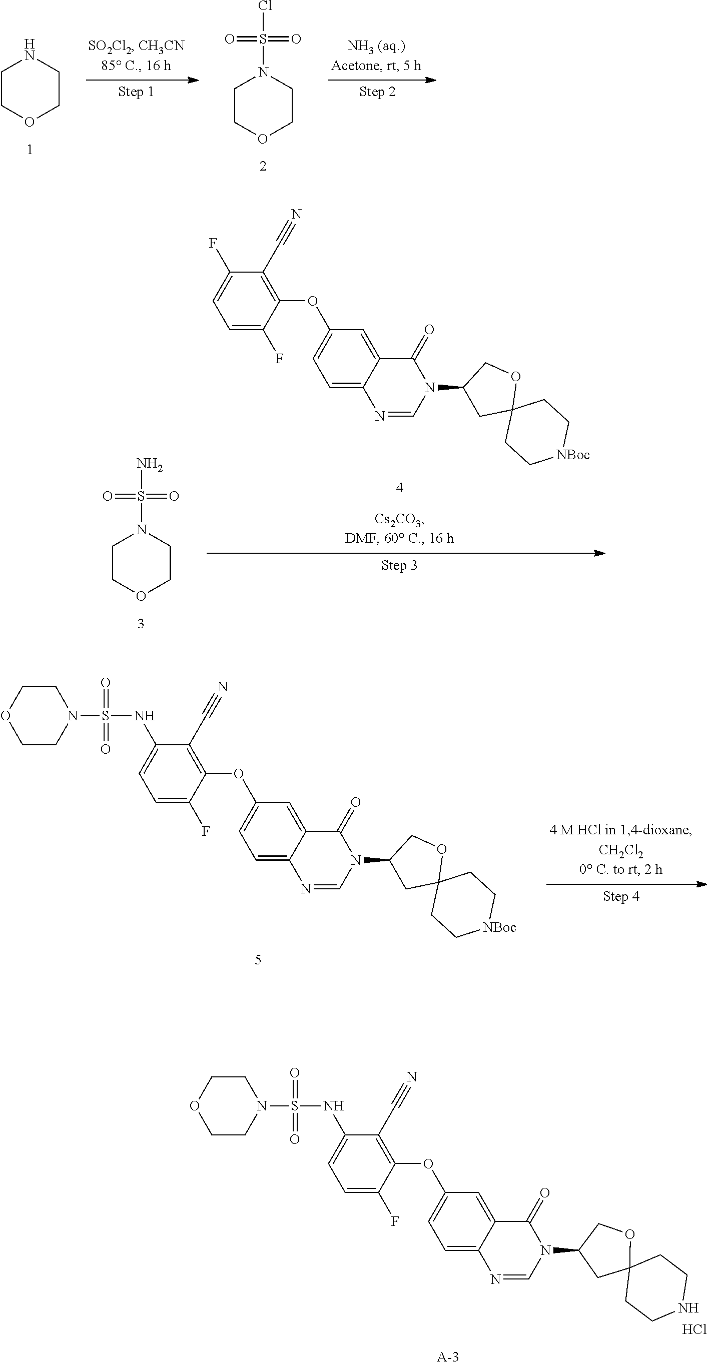

### Step 1

Into a 250 mL single-neck round bottom flask containing a well-stirred solution of morpholine (1, 57.39 mmol, 5.02 mL) in acetonitrile (40 mL) was added sulfuryl chloride (172.18 mmol, 13.96 mL) at 0° C. Resulting reaction mixture was heated at 85° C. for 16 hours. After completion of the reaction, the reaction mixture was concentrated under reduced pressure and co-distilled with toluene (2×50 mL) to afford morpholine-4-sulfonyl chloride (2, 8 g, 30.17 mmol, 53% yield) as a pale brown colored solid. ¹H NMR (400 MHz, DMSO-d₆): δ 3.79, 3.28.

### Step 2

Into a 250 mL single-neck round-bottom flask containing a well-stirred solution of morpholine-4-sulfonyl chloride (2, 8 g, 30.17 mmol) in acetone (15 mL) was added ammonium hydroxide (30% solution) (616.34 mmol, 24 mL) at 0° C. Resulting reaction mixture was stirred at room temperature for 5 hours. After completion of the reaction, the reaction mixture was concentrated under reduced pressure and co-distilled with toluene (2×50 mL) to afford crude residue which was purified by flash (silica-gel 230-400 mesh, 100 g) column chromatography using 0-10% methanol/dichloromethane as an eluent to afford morpholine-4-sulfonamide (3, 3 g, 17.15 mmol, 57% yield) as an off-white solid. ¹H NMR (400 MHz, DMSO-d₆): δ 6.83 (s, 2H), 3.65 (t, J=4.80 Hz, 4H), 2.92 (t, J=4.80 Hz, 4H)

### Step 3

Into a 40 mL pressure vial containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (4, 300 mg, 543.97 mol) in N,N-dimethylformamide (6 mL) were added morpholine-4-sulfonamide (3, 190.33 mg, 1.09 mmol) and Cs₂CO₃ (531.71 mg, 1.63 mmol) at room temperature. Reaction mixture was stirred at 60° C. for 16 hours. After completion of the reaction, the mixture was quenched with water (5 mL) and extracted with ethyl acetate (3×15 mL). Combined organic layer was washed with water (10 mL), saturated NaCl solution (10 mL), dried over anhydrous sodium sulfate, filtered and concentrated under reduced pressure to afford crude residue which was purified by flash (silica gel 230-400 mesh, 25 g) column chromatography using 0-100% ethyl acetate/hexane as an eluent to afford tert-butyl(3R)-3-[6-[2-cyano-6-fluoro-3-(morpholinosulfonylamino)phenoxy]-4-oxo-quinazolin-3-yl]-1-oxa-8-azaspiro[4.5]decane-8-carboxylate (5, 240 mg, 298.87 mol, 55% yield) as an off-white solid. UPLC-MS (ES+): m/z 629.5 [M-^{t}Bu+H]⁺.

### Step 4

Into a 50 mL single-neck round-bottom flask containing a well-stirred solution of tert-butyl(3R)-3-[6-[2-cyano-6-fluoro-3-(morpholinosulfonylamino)phenoxy]-4-oxo-quinazolin-3-yl]-1-oxa-8-azaspiro[4.5]decane-8-carboxylate (5, 270 mg, 336.23 mol) in dichloromethane (6 ml) was added 4M HCl in 1,4-dioxane (336.23 mol, 3 mL) at 0° C. The resulting reaction mixture was stirred at room temperature for 2 hours. After completion of the reaction, the mixture was concentrated under reduced pressure and co-distilled with toluene (10 mL) to afford N-[2-cyano-4-fluoro-3-[3-[(3R)-1-oxa-8-azaspiro[4.5]decan-3-yl]-4-oxo-quinazolin-6-yl]oxy-phenyl]morpholine-4-sulfonamide (A-3, 220 mg, 314.09 mol, 93% yield, HCl salt) as an off-white solid. UPLC-MS (ES+): m/z 585.2 [M+H]⁺.

### Synthesis A4: Synthesis of N-[2-cyano-4-fluoro-3-[3-[(3R)-1-oxa-8-azaspiro[4.5]decan-3-yl]-4-oxo-quinazolin-6-yl]oxy-phenyl]tetrahydropyran-4-sulfonamide (A-4)

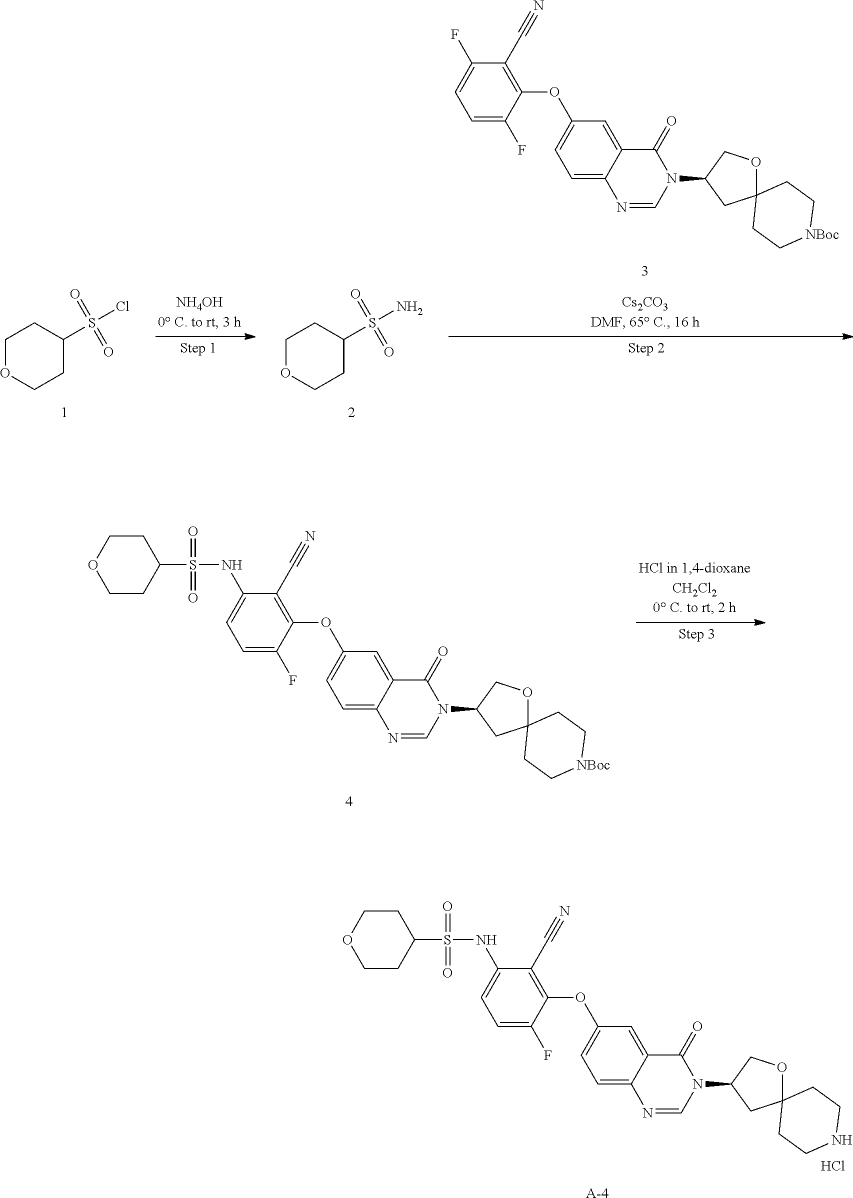

### Step 1

Into a 50 mL single-neck round-bottom flask containing a well-stirred solution of tetrahydropyran-4-sulfonyl chloride (1, 200 mg, 1.08 mmol) in acetone (5 mL) was added ammonia solution 25% (1.08 mmol, 2 mL) at room temperature under nitrogen atmosphere and stirred at room temperature for 3 hours. After completion of the reaction, the mixture was concentrated under reduced pressure to get a crude residue which was purified by flash silica-gel (230-400 mesh) column using 0-100% ethyl acetate/hexane as an eluentto afford tetrahydropyran-4-sulfonamide (2, 165 mg, 898.85 mol, 83% yield) as an off-white solid. ¹H NMR (400 MHz, DMSO-d₆): δ 6.75 (bs, 2H), 3.94 (dd, J=4.00, 11.20 Hz, 2H), 3.35-3.29 (m, 2H), 3.10-3.02 (m, 1H), 1.91-1.87 (m, 2H), 1.66-1.55 (m, 2H).

### Step 2

Into a 20 mL pressure vial containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (3, 350 mg, 649.90 mol) in N,N-dimethylformamide (5 mL) were added Cs₂CO₃ (529.38 mg, 1.62 mmol) and tetrahydropyran-4-sulfonamide (2, 161.06 mg, 974.86 mol) at room temperature. Resulting reaction mixture was stirred at 65° C. for 16 hours. After completion of the reaction, the mixture was quenched with water (50 mL) and extracted with ethyl acetate (3×100 mL). Combined organic layer was washed with water (50 mL), saturated NaCl solution (50 mL), dried over anhydrous Na₂SO₄, filtered and concentrated under reduced pressure to afford a crude residue which was purified by flash column chromatography (25 g, 230-400 silica-gel) using 0-100% ethyl acetate/hexane as an eluent to afford tert-butyl(3R)-3-[6-[2-cyano-6-fluoro-3-(tetrahydropyran-4-ylsulfonylamino)phenoxy]-4-oxo-quinazolin-3-yl]-1-oxa-8-azaspiro[4.5]decane-8-carboxylate (4, 230 mg, 329.55 mol, 51% yield) as an off-white solid. UPLC-MS: m/z 682.0 [M+H]⁺.

### Step 3

Into a 25 mL single-neck round-bottom flask containing a well-stirred solution of tert-butyl(3R)-3-[6-[2-cyano-6-fluoro-3-(tetrahydropyran-4-ylsulfonylamino)phenoxy]-4-oxo-quinazolin-3-yl]-1-oxa-8-azaspiro[4.5]decane-8-carboxylate (4, 230 mg, 329.65 mmol) in dichloromethane (5 mL) was added HCl in 1,4-dioxane (4.0 M, 1.00 mL) dropwise at 0° C. under nitrogen atmosphere and reaction mixture was stirred at room temperature for 2 hours. After completion of the reaction, the mixture was concentrated under reduced pressure to get a crude which was co-distilled with hexane (2×10 mL). Obtained crude material was triturated with methyl tert-butyl ether (2×5 mL) and dried under reduced pressure to afford N-[2-cyano-4-fluoro-3-[3-[(3R)-1-oxa-8-azaspiro[4.5]decan-3-yl]-4-oxo-quinazolin-6-yl]oxy-phenyl]tetrahydropyran-4-sulfonamide (A-4, 200 mg, 317.02 mol, 10% yield, HCl salt) as an off-white solid. UPLC-MS (ES+): m/z 584.4 [M+H]⁺.

### Synthesis A5: Synthesis of N-[2-cyano-4-fluoro-3-[3-[(3R)-1-oxa-8-azaspiro[4.5]decan-3-yl]-4-oxo-quinazolin-6-yl]oxy-phenyl]-3-azabicyclo[3.1.0]hexane-3-sulfonamide (A-5)

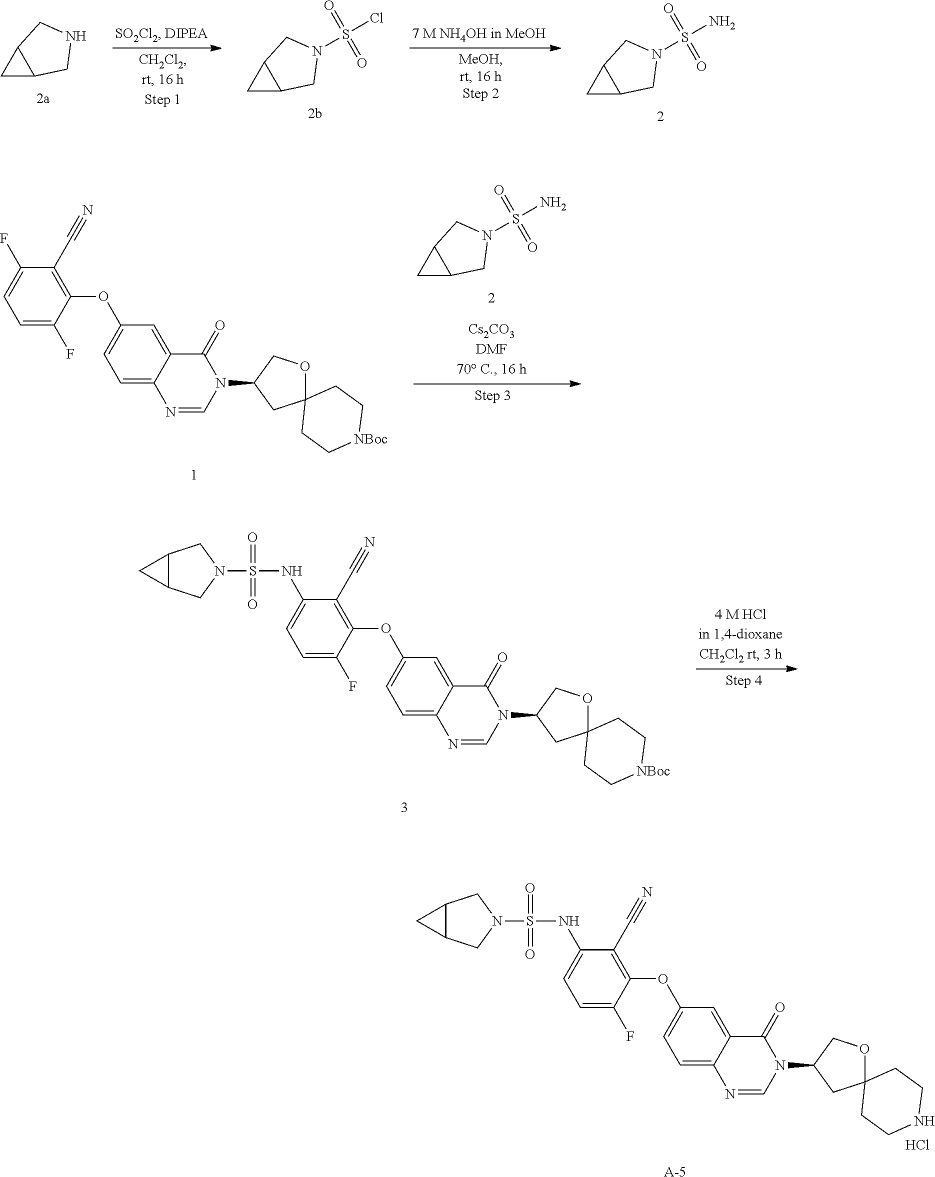

### Step 1

Into a 100 mL two-neck round bottom flask containing a well stirred solution of 3-azabicyclo[3.1.0]hexane (2a, 1.0 g, 12.03 mmol) in dichloromethane (20 mL) were added N,N-diisopropylethylamine (18.04 mmol, 3.14 mL) and sulfuryl chloride (30.07 mmol, 2.44 mL) at 0° C. Resulting reaction mixture was stirred at room temperature for 16 hours. After completion of the reaction, the mixture was quenched with water (20 mL) at 0° C. and extracted with ethyl acetate (3×30 mL). Combined organic layer was washed with 1.5 N HCl solution (2×10 mL), dried over anhydrous Na₂SO₄, filtered and concentrated under reduced pressure to afford 3-azabicyclo[3.1.0]hexane-3-sulfonyl chloride (2b, 1.6 g, 8.81 mmol, 73% yield) as a light yellow semi solid. ¹H NMR (400 MHz, DMSO-d₆): δ 1.83-1.68 (m, 4H), 0.85-0.72 (m, 2H), 0.49-0.32 (m, 2H).

### Step 2

Into a 100 mL two-neck round bottom flask containing a well stirred solution of 3-azabicyclo[3.1.0]hexane-3-sulfonyl chloride (2b, 1.6 g, 8.81 mmol) in methanol (20 mL) was added 7 M ammonia in methanol (7 M, 16 mL) at 0° C. and resulting reaction mixture was and stirred at room temperature for 16 hours. After completion of the reaction, the reaction mixture was quenched with water (50 mL) at 0° C. and extracted with ethyl acetate (2×50 mL). Combined organic layer was washed with NaHCO₃ solution (30 mL), dried over anhydrous Na₂SO₄, filtered and concentrated under reduced pressure to get a crude residue which was triturated with hexane (10 mL) and dried under reduced pressure to afford 3-azabicyclo[3.1.0]hexane-3-sulfonamide (2, 0.700 g, 4.27 mmol, 49% yield) as a yellow solid. UPLC-MS (ES+): m/z 163.0 [M+H]⁺.

### Step 3

Into a 40 mL pressure glass vial containing a well stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (1, 0.300 g, 557.06 mol) and 3-azabicyclo[3.1.0]hexane-3-sulfonamide (2, 361.44 mg, 2.23 mmol) in N,N-dimethylformamide (5 mL) was added Cs₂CO₃ (544.50 mg, 1.67 mmol) at room temperature under nitrogen atmosphere. Resulting reaction mixture was stirred at 70° C. for 16 hours. After completion of the reaction, the mixture was diluted with water (20 mL) and extracted with ethyl acetate (2×20 mL). Combined organic layer was washed with saturated NaCl (10 mL), dried over anhydrous sodium sulphate, filtered and concentrated under reduced pressure to get crude residue which was purified by flash (25 g, 230-400 silica-gel mesh) column chromatography using 0-100% ethyl acetate in hexane as an eluent to afford tert-butyl(3R)-3-[6-[3-(3-azabicyclo[3.1.0]hexan-3-ylsulfonylamino)-2-cyano-6-fluoro-phenoxy]-4-oxo-quinazolin-3-yl]-1-oxa-8-azaspiro[4.5]decane-8-carboxylate (3, 0.3 g, 409.85 mol, 74% yield) as an off-white solid. UPLC-MS (ES+): m/z 681.5 [M+H]⁺.

### Step 4

Into a 50 mL single-neck round bottom flask containing a well stirred solution of tert-butyl(3R)-3-[6-[3-(3-azabicyclo[3.1.0]hexan-3-ylsulfonylamino)-2-cyano-6-fluoro-phenoxy]-4-oxo-quinazolin-3-yl]-1-oxa-8-azaspiro[4.5]decane-8-carboxylate (3, 440.69 mol, 1.2 mL) in dichloromethane (5 mL) was added 4 M HCl in dioxane (440.69 mol, 1.2 mL) at 0° C. under nitrogen atmosphere. Resulting reaction mixture was stirred at room temperature for 3 hours. After completion of the reaction, the mixture was concentrated under reduced pressure to afford N-[2-cyano-4-fluoro-3-[3-[(3R)-1-oxa-8-azaspiro[4.5]decan-3-yl]-4-oxo-quinazolin-6-yl]oxy-phenyl]-3-azabicyclo[3.1.0]hexane-3-sulfonamide (A-5, 0.280 g, 435.59 mol, 99% yield, HCl salt) as a brown liquid gummy. UPLC-MS (ES+): m/z 581.2 [M+H]⁺.

### Synthesis A6: Synthesis of N-[2-cyano-4-fluoro-3-[3-[(3R)-1-oxa-8-azaspiro[4.5]decan-3-yl]-4-oxo-quinazolin-6-yl]oxy-phenyl]-3-methoxy-azetidine-1-sulfonamide (A-6)

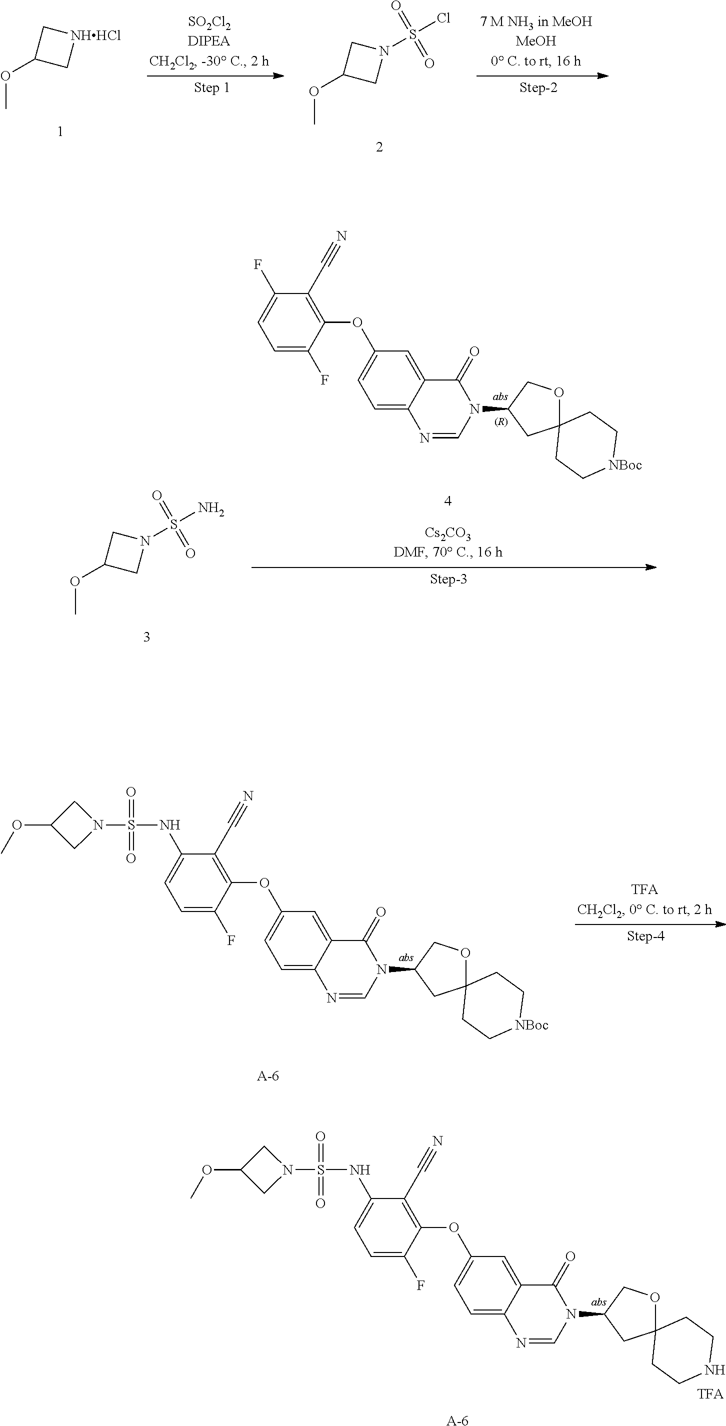

### Step 1

Into a 100 mL two-neck round-bottom flask containing a well stirred solution of 3-methoxyazetidine (1, 3 g, 24.28 mmol, HCl salt) in anhydrous dichloromethane (30 mL) was added N,N-diisopropylethylamine (48.55 mmol, 8.46 mL) followed by dropwise addition of sulfuryl chloride (48.55 mmol, 3.94 mL) at −30° C. under nitrogen atmosphere. After addition, reaction mixture was stirred at −30° C. for 2 hours. After completion of reaction, the mixture was quenched with cold water (50 mL) and extracted with dichloromethane (2×60 mL). Combined organic layers were washed with water (50 mL), saturated NaCl solution (50 mL), and the organic layer was dried over anhydrous Na₂SO₄, filtered, and filtrate was concentrated under reduced pressure to afford 3-methoxyazetidine-1-sulfonyl chloride (2, 2.7 g, 14.53 mmol, 60% yield) as a colorless liquid. UPLC-MS (ES+): m/z 129.5 [M+−^{t}Bu]⁺.

### Step 2

Into a 250 mL two-neck round bottom flask containing a well-stirred solution of 3-methoxyazetidine-1-sulfonyl chloride (2, 2.7 g, 14.55 mmol) in methanol (30 mL) was added 7 N ammonia in methanol (247.71 mg, 14.55 mmol) at 0° C. and stirred at room temperature for 16 hours. After completion of the reaction, the reaction mixture was concentrated under reduced pressure to get crude which was triturated with hexane (2×10 mL), concentrated under reduced pressure to afford 3-methoxyazetidine-1-sulfonamide (3, 2.3 g, 13.84 mmol, 95% yield) as an off-white solid. ¹H NMR (400 MHz, DMSO-d₆): δ 6.95 (s, 2H), 4.09 (m, 1H), 3.83-3.79 (m, 2H), 3.59-3.56 (m, 2H), 3.20 (s, 3H).

### Step 3

Into a 40 mL pressure vial containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (4, 500 mg, 928.43 mol) in N,N-dimethylformamide (10 mL) were added 3-methoxyazetidine-1-sulfonamide (3, 231.46 mg, 1.39 mmol) and Cs₂CO₃ (907.51 mg, 2.79 mmol) at room temperature and stirred at 70° C. for 16 hours. After completion of the reaction, the mixture was diluted with water (30 mL) and filtered through Buchner funnel to remove the fluorescent impurity. The filtrate was extracted with ethyl acetate (2×50 mL) and combined organic layers were washed with cold water (2×20 mL), dried over anhydrous Na₂SO₄, filtered and filtrate was concentrated under reduced pressure to afford tert-butyl(3R)-3-[6-[2-cyano-6-fluoro-3-[(3-methoxyazetidin-1-yl)sulfonylamino]phenoxy]-4-oxo-quinazolin-3-yl]-1-oxa-8-azaspiro[4.5]decane-8-carboxylate (5, 450 mg, 542.12 mol, 58% yield) as a brown gummy liquid. UPLC-MS (ES+): m/z 685.2 [M+H]⁺.

### Step 4

Into a 25 mL single-neck round-bottom flask containing a well-stirred solution of tert-butyl(3R)-3-[6-[2-cyano-6-fluoro-3-[(3-methoxyazetidin-1-yl)sulfonylamino]phenoxy]-4-oxo-quinazolin-3-yl]-1-oxa-8-azaspiro[4.5]decane-8-carboxylate (5, 450 mg, 542.12 mol) in anhydrous dichloromethane (5 mL) was added trifluoroacetic acid (26.14 mmol, 2.0 mL) slowly at 0° C. under nitrogen atmosphere. The resulting reaction mixture was stirred at room temperature for 2 hours. After completion of the reaction, the reaction mixture was concentrated under reduced pressure, which was triturated with methyl tert-butyl ether (2×2 mL) to remove the non-polar impurities and dried under reduced pressure to afford N-[2-cyano-4-fluoro-3-[3-[(3R)-1-oxa-8-azaspiro[4.5]decan-3-yl]-4-oxo-quinazolin-6-yl]oxy-phenyl]-3-methoxy-azetidine-1-sulfonamide (A-6, 420 mg, 535.40 mol, 99% yield, TFA salt) as an off-white solid. UPLC-MS (ES+): m/z 585.1 [M+H]⁺

### Synthesis A7: Synthesis of N-[2-cyano-4-fluoro-3-[3-[(3R)-1-oxa-8-azaspiro[4.5]decan-3-yl]-4-oxo-quinazolin-6-yl]oxy-phenyl]-2-azaspiro[3.3]heptane-2-sulfonamide (A-7)

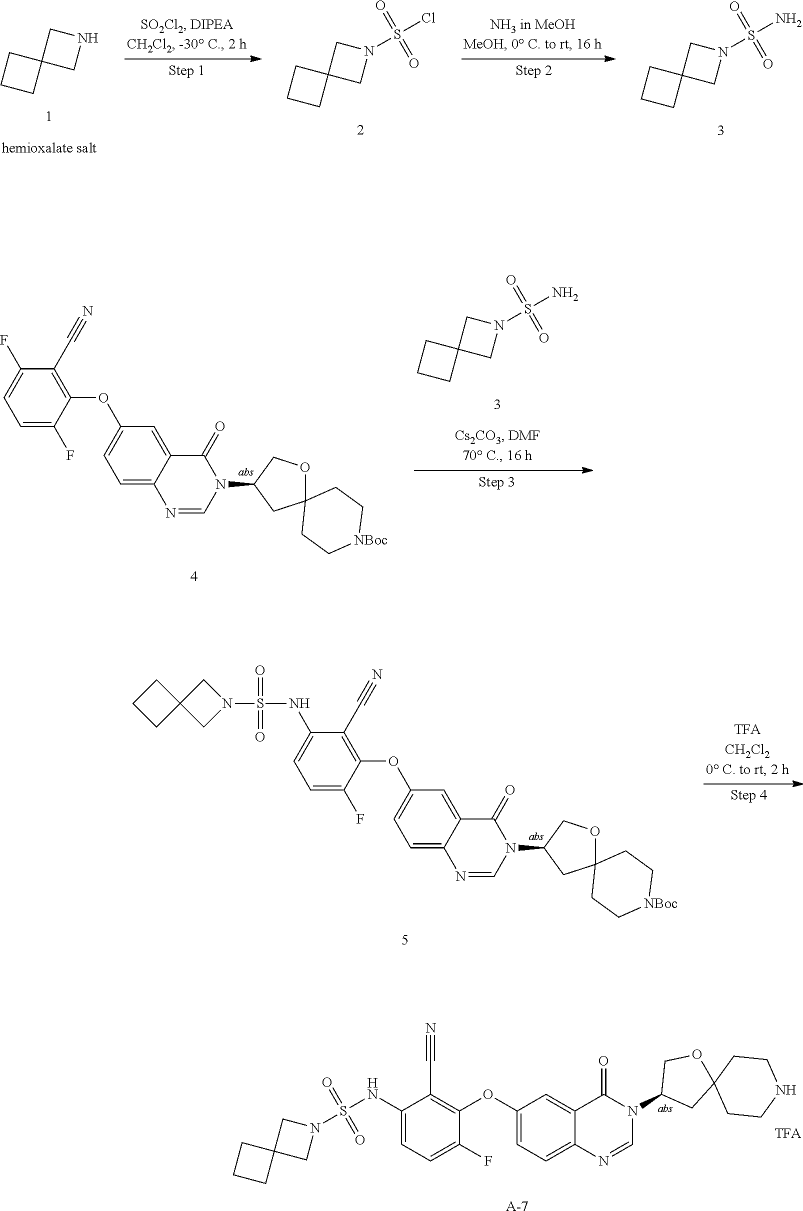

### Step 1

Into a 250 mL two neck round bottom flask containing a well stirred solution of 2-azaspiro[3.3]heptane (Hemioxalate salt) (1, 3 g, 30.88 mmol) in dichloromethane (30.00 mL) were added sulfuryl chloride (185.27 mmol, 15.02 mL), N,N-diisopropylethylamine (92.63 mmol, 16.13 mL) at −30° C. and stirred at same reaction condition for 2 hours. After completion of the reaction, the reaction mixture was quenched by dropwise addition of water (50 mL), extracted with ethyl acetate (2×50 mL). Combined organic layers washed with 1.5 N HCl solution (2×50 mL), organic layers dried over anhydrous Na₂SO₄, filtered and concentrated under reduced pressure to afford crude 2-azaspiro[3.3]heptane-2-sulfonyl chloride (2, 2.4 g, 12.14 mmol, 39% yield) as a light brown liquid. ¹H NMR (400 MHz, DMSO-d₆): δ 4.19-4.02 (m, 4H), 2.31-2.09 (m, 4H), 1.81-1.73 (m, 2H).

### Step 2

Into a 100 mL sealed-tube containing a well-stirred solution of 2-azaspiro[3.3]heptane-2-sulfonyl chloride (2, 2.4 g, 12.27 mmol) in methanol (30 mL) was added ammonia 7 M in methanol (7 M, 15.77 mL) at 0° C. and stirred the reaction mixture at room temperature for 16 hours. After completion of the reaction, the reaction mixture was concentrated under reduced pressure, diluted with water (50 mL), and extracted with ethyl acetate (3×50 mL). The organic layer was washed with NaHCO₃ solution (50 mL), dried over anhydrous Na₂SO₄, and concentrated under reduced pressure. The crude product was washed with n-hexane (3×25 mL) to afford 2-azaspiro[3.3]heptane-2-sulfonamide (3, 1.2 g, 6.74 mmol, 55% yield) as a brown solid. ¹H NMR (400 MHz, DMSO-d₆): δ 6.81 (s, 2H), 3.76 (s, 4H), 2.09 (t, J=8.00 Hz, 4H), 1.82-1.75 (m, 2H).

### Step 3

Into a 20 mL pressure vial containing a well stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (4, 100 mg, 181.97 mol) in N,N-diisopropylethylamine (3 mL) were added Cs₂CO₃ (177.87 mg, 545.92 mol) and 2-azaspiro[3.3]heptane-2-sulfonamide (3, 64.79 mg, 363.95 mol) at room temperature and the reaction mixture was stirred at 70° C. for 16 hours. After completion of the reaction, the reaction mixture was diluted with ethyl acetate (10 mL), washed with water (10 mL), saturated NaCl solution (10 mL), dried over anhydrous Na₂SO₄, and concentrated under reduced pressure to get a crude residue which was purified by flash silica-gel (230-400 mesh) column chromatography with 0-100% ethyl acetate/hexane to afford tert-butyl(3R)-3-[6-[3-(2-azaspiro[3.3]heptan-2-ylsulfonylamino)-2-cyano-6-fluoro-phenoxy]-4-oxo-quinazolin-3-yl]-1-oxa-8-azaspiro[4.5]decane-8-carboxylate (5, 90 mg, 80.31 mol, 44% yield) as an off-white solid. UPLC-MS (ES+): m/z 695.3 [M+H]⁺.

### Step 4

Into a 25 mL two neck round-bottom flask containing a stirred solution of tert-butyl(3R)-3-[6-[3-(2-azaspiro[3.3]heptan-2-ylsulfonylamino)-2-cyano-6-fluoro-phenoxy]-4-oxo-quinazolin-3-yl]-1-oxa-8-azaspiro[4.5]decane-8-carboxylate (5, 90 mg, 85.50 mol) in dichloromethane (2 mL) was added trifluoroacetic acid (1.71 mmol, 130.85 L) at 0° C. The reaction was stirred at room temperature for 2 hours. After completion of the reaction, the reaction mixture was concentrated under reduced pressure to get a crude residue which was washed with methyl tert-butyl ether (2×5 mL) and dried under reduced pressure to afford N-[2-cyano-4-fluoro-3-[3-[(3R)-1-oxa-8-azaspiro[4.5]decan-3-yl]-4-oxo-quinazolin-6-yl]oxy-phenyl]-2-azaspiro[3.3]heptane-2-sulfonamide (A-7, 90 mg, 68.58 mol, 80% yield, TFA salt) as an off-white solid. UPLC-MS (ES+): m/z 595.2 [M+H]⁺.

### Synthesis A8: Synthesis of N-[2-cyano-4-fluoro-3-[3-[(3R)-1-oxa-8-azaspiro[4.5]decan-3-yl]-4-oxo-quinazolin-6-yl]oxy-phenyl]-5-azaspiro[2.4]heptane-5-sulfonamide (A-8)

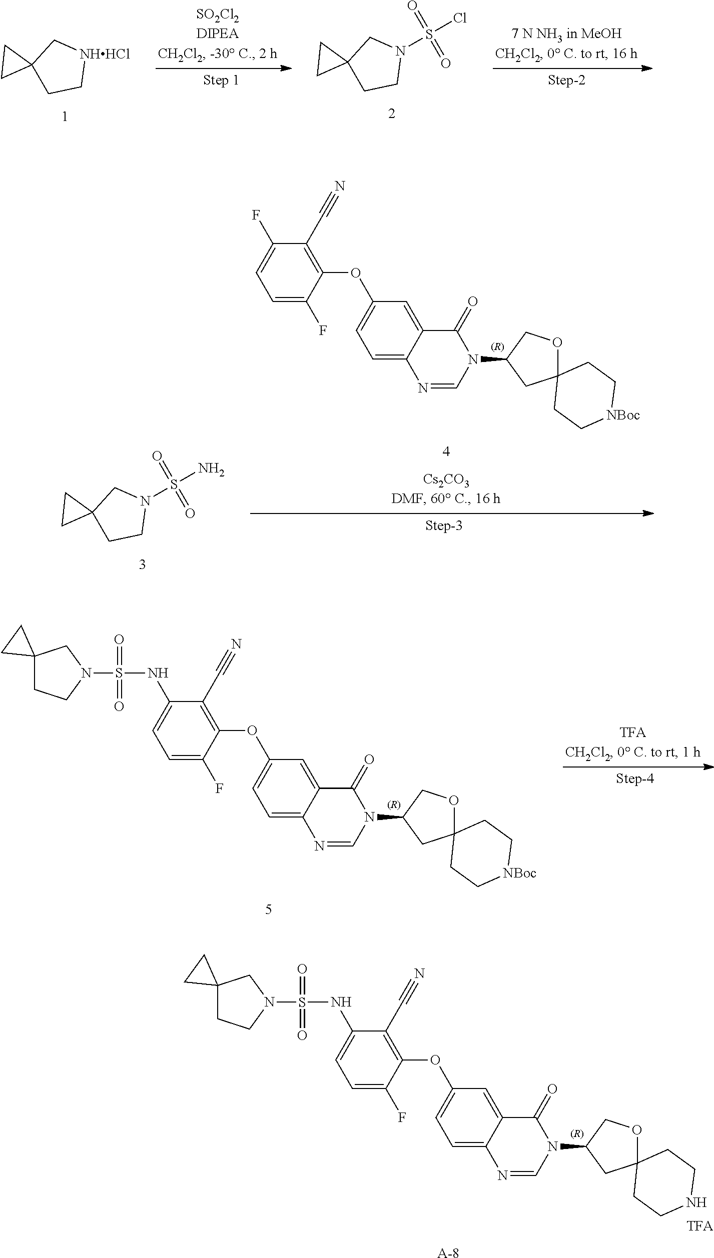

### Step 1

Into a 100 mL two-neck round-bottom flask containing a stirred solution of 5-azaspiro[2.4]heptane (1, 1.0 g, 7.48 mmol, HCl salt) in anhydrous dichloromethane (15 mL) was added N,N-diisopropylethylamine (11.23 mmol, 1.96 mL) at −30° C. followed by dropwise addition of a prepared solution of sulfuryl chloride (7.48 mmol, 606.67 μL) in anhydrous dichloromethane (2 mL). After addition reaction mixture was stirred at −30° C. to −50° C. for 2 hours. After completion of reaction, the reaction mixture was diluted with cold water (20 mL) and extracted with dichloromethane (2×30 mL). Combined organic layers were dried over Na₂SO₄, filtered and filtrate was concentrated under reduced pressure to get a crude residue which was purified by flash silica-gel (60-120 mesh) column with 0-100% ethyl acetate/hexane to afford 5-azaspiro[2.4]heptane-5-sulfonyl chloride (2, 690 mg, 3.53 mmol, 47% yield) as a colorless liquid. ¹H NMR (400 MHz, DMSO-d₆): δ 3.61 (t, J=6.80 Hz, 2H), 3.39 (m, 2H), 1.92 (t, J=7.20 Hz, 2H), 0.73-0.69 (m, 2H), 0.66-0.63 (m, 2H).

### Step 2

Into a 100 mL single-neck round-bottom flask containing a well stirred solution of 5-azaspiro[2.4]heptane-5-sulfonyl chloride (2, 690 mg, 3.53 mmol) in methanol (7 mL) was added dropwise 7 N ammonia in methanol (7 M, 10.08 mL) at 0° C. After addition, the reaction mixture was stirred at room temperature for 16 hours. After completion of the reaction, the reaction mixture was concentrated under reduced pressure to get a crude, which was triturated with hexane (2×10 mL) and concentrated under reduced pressure to afford 5-azaspiro[2.4]heptane-5-sulfonamide (3, 600 mg, 3.40 mmol, 97% yield) as an off-white solid. Crude product was taken to the next step without further purification. ¹H NMR (400 MHz, DMSO-d₆): δ 6.73 (s, 2H), 3.26 (t, J=6.80 Hz, 2H), 3.05-3.02 (m, 2H), 1.76 (t, J=6.80 Hz, 2H), 0.61-0.58 (m, 2H), 0.55-0.52 (m, 2H).

### Step 3

Into a 50 mL single-neck round-bottom flask containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (4, 500 mg, 928.43 mol) and 5-azaspiro[2.4]heptane-5-sulfonamide (3, 327.25 mg, 1.86 mmol) in N,N-dimethylformamide (10 mL) was added Cs₂CO₃ (756.26 mg, 2.32 mmol) at room temperature and the resulting suspension was stirred at 60° C. for 16 hours under nitrogen atmosphere. After completion of the reaction, the reaction mixture was diluted with water (30 mL) and extracted with ethyl acetate (2×50 mL). Combined organic layers were dried over anhydrous Na₂SO₄, filtered and filtrate was concentrated under reduced pressure to get a crude residue which was purified by flash silica-gel (230-400 mesh) column chromatography with 0-100% ethyl acetate/hexane to afford impure product. The impure product was again re-purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fraction was lyophilized to afford tert-butyl(3R)-3-[6-[3-(5-azaspiro[2.4]heptan-5-ylsulfonylamino)-2-cyano-6-fluoro-phenoxy]-4-oxo-quinazolin-3-yl]-1-oxa-8-azaspiro[4.5]decane-8-carboxylate (5, 200 mg, 284.99 mol, 31% yield) as an off-white solid. UPLC-MS (ES+): m/z 695.2 [M+H]⁺.

### Step 4

Into a 50 mL single-neck round-bottom flask containing a well-stirred solution of tert-butyl(3R)-3-[6-[3-(5-azaspiro[2.4]heptan-5-ylsulfonylamino)-2-cyano-6-fluoro-phenoxy]-4-oxo-quinazolin-3-yl]-1-oxa-8-azaspiro[4.5]decane-8-carboxylate (5, 190 mg, 273.47 mol) in anhydrous dichloromethane (2 mL) was added trifluoroacetic acid (11.76 mmol, 0.9 mL) at 0° C. under nitrogen atmosphere. The resulting reaction mixture was stirred at room temperature for 1 hour. After completion of the reaction, the reaction mixture was concentrated under reduced pressure and triturated with methyl tert-butyl ether (10 mL) to afford N-[2-cyano-4-fluoro-3-[3-[(3R)-1-oxa-8-azaspiro[4.5]decan-3-yl]-4-oxo-quinazolin-6-yl]oxy-phenyl]-5-azaspiro[2.4]heptane-5-sulfonamide (A-8, 190 mg, 265.42 mol, 97% yield, TFA salt) as an brown gum. Crude was used for the next step without further purification. UPLC-MS (ES+): m/z 595.2 [M+H]⁺.

### Synthesis A9: Synthesis of (3R)-3-[6-[2-cyano-6-fluoro-3-[[2-hydroxyethyl(methyl)sulfamoyl]amino]phenoxy]-4-oxo-quinazolin-3-yl]-1-oxa-8-azaspiro[4.5]decane (A-9)

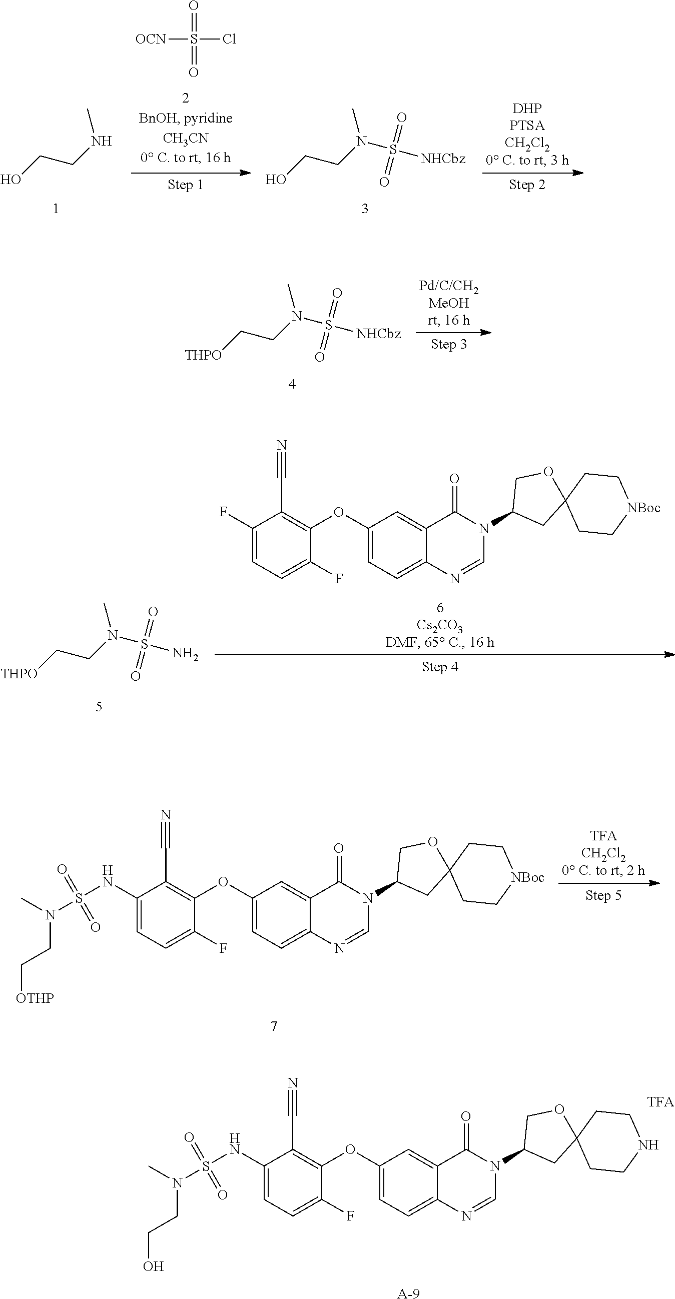

### Step 1

Into a 1000 mL three-neck round bottom flask containing a well stirred solution of N-(oxomethylene)sulfamoyl chloride (2, 9.42 g, 66.58 mmol, 5.80 mL) in acetonitrile (400 mL) was added benzyl alcohol (55.48 mmol, 5.74 mL) at 0° C. and the reaction mixture was stirred at the same temperature for 30 min. To the above mixture, pyridine (66.58 mmol, 5.36 mL) was added at 0° C. and mixture was stirred for 30 minutes. Finally, 2-(methylamino)ethanol (1, 221.94 mmol, 17.75 mL) was added and the resulting reaction mixture was stirred for 16 hours at room temperature. After completion of the reaction, the reaction mixture was quenched with 1M HCl solution and extracted with ethyl acetate (3×100 mL). Combined organic layers were washed with saturated NaCl solution, dried over anhydrous Na₂SO₄ and concentrated under reduced pressure to afford benzyl N-[2-hydroxyethyl(methyl)sulfamoyl]carbamate (3, 14 g, 48.33 mmol, 87% yield) as colorless viscous liquid. UPLC-MS (ES+): m/z 289.0 [M+H]⁺.

### Step 2

To a 500 mL single neck round-bottom flask containing a stirred solution of benzyl N-[2-hydroxyethyl(methyl)sulfamoyl]carbamate (3, 6 g, 20.60 mmol) in dichloromethane (100 mL) were added p-toluenesulfonic acid monohydrate (391.89 mg, 2.06 mmol) and 3,4-dihydro-2H-pyran (2.60 g, 30.90 mmol, 2.81 mL) at 0° C. and the reaction mixture was stirred at room temperature for 3 hours. After the completion of the reaction, the reaction mixture was diluted with the water (100 mL) and extracted with dichloromethane (3×100 mL). The combined organic layer was washed with saturated NaCl solution (100 mL), dried over anhydrous Na₂SO₄, and concentrated to get benzyl N-[methyl(2-tetrahydropyran-2-yloxyethyl)sulfamoyl]carbamate (4, 6 g, 15.33 mmol, 74% yield) was obtained as a pale yellow oil, which was used as such in the next reaction without further purification. UPLC-MS (ES−): m/z 371.1 [M−H]⁻.

### Step 3

Into a 500 mL two-neck round-bottom flask containing a stirred solution of benzyl N-[methyl(2-tetrahydropyran-2-yloxyethyl)sulfamoyl]carbamate (4, 6 g, 15.30 mmol) in methanol (120 mL) was added palladium, 10% on carbon, Type 487, dry (570.00 mg, 5.36 mmol) and the reaction mixture was stirred under hydrogen (bladder) atmosphere at room temperature for 16 hours. After the completion of the reaction, the reaction mixture was filtered through a celite bed and filtrate was concentrated under reduced pressure to afford 2-[2-[methyl(sulfamoyl)amino]ethoxy]tetrahydropyran (5, 2.6 g, 10.58 mmol, 69% yield) as a colorless oil which was used as such in the next step. UPLC-MS (ES−): m/z 237.2 [M−H]⁻.

### Step 4

Into a 250 mL two-neck round-bottom flask attached with a reflux condenser containing a stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (6, 2.00 g, 3.28 mmol) in N,N-dimethylformamide (40 mL) were added and Cs₂CO₃ (3.21 g, 9.84 mmol) and 2-[2-[methyl(sulfamoyl)amino]ethoxy]tetrahydropyran (5, 1.61 g, 6.56 mmol) at room temperature and the reaction mixture was heated at 65° C. for 16 hours. The reaction mixture was diluted with ethyl acetate (100 mL), washed with water (100 mL), saturated NaCl solution (100 mL), dried over anhydrous Na₂SO₄, and concentrated under reduced pressure to get a crude residue which was purified by flash silica gel (230-400 mesh) column chromatography to afford tert-butyl(3R)-3-[6-[2-cyano-6-fluoro-3-[[methyl(2-tetrahydropyran-2-yloxyethyl)sulfamoyl]amino]phenoxy]-4-oxo-quinazolin-3-yl]-1-oxa-8-azaspiro[4.5]decane-8-carboxylate (7, 2 g, 1.98 mmol, 60% yield) as a colorless gum. UPLC-MS (ES+): m/z 757.4 [M+H]⁺.

### Step 5

Into a 100 mL two-neck round-bottom flask containing a solution of tert-butyl(3R)-3-[6-[2-cyano-6-fluoro-3-[[methyl(2-tetrahydropyran-2-yloxyethyl)sulfamoyl]amino]phenoxy]-4-oxo-quinazolin-3-yl]-1-oxa-8-azaspiro[4.5]decane-8-carboxylate (7, 2.00 g, 1.98 mmol) in dichloromethane (20 mL) was added trifluoroacetic acid (26.14 mmol, 2 mL) at 0° C. Reaction was stirred at room temperature for 2 hours. After complete consumption of starting material, the reaction mixture was concentrated under reduced pressure and obtained residue was triturated with methyl tert-butyl ether (2×20 mL) and dried under vacuum to obtain (3R)-3-[6-[2-cyano-6-fluoro-3-[[2-hydroxyethyl(methyl)sulfamoyl]amino]phenoxy]-4-oxo-quinazolin-3-yl]-1-oxa-8-azaspiro[4.5]decane (A-9, 2 g, 1.82 mmol, 92% yield, TFA salt) as a brown solid, which was used without further purification in the next step. UPLC-MS (ES+): m/z 573.2 [M+H]⁺.

### Synthesis A10: Synthesis of N-[2-cyano-4-fluoro-3-[3-[(3R)-1-oxa-8-azaspiro[4.5]decan-3-yl]-4-oxo-quinazolin-6-yl]oxy-phenyl]propane-2-sulfonamide (A-10)

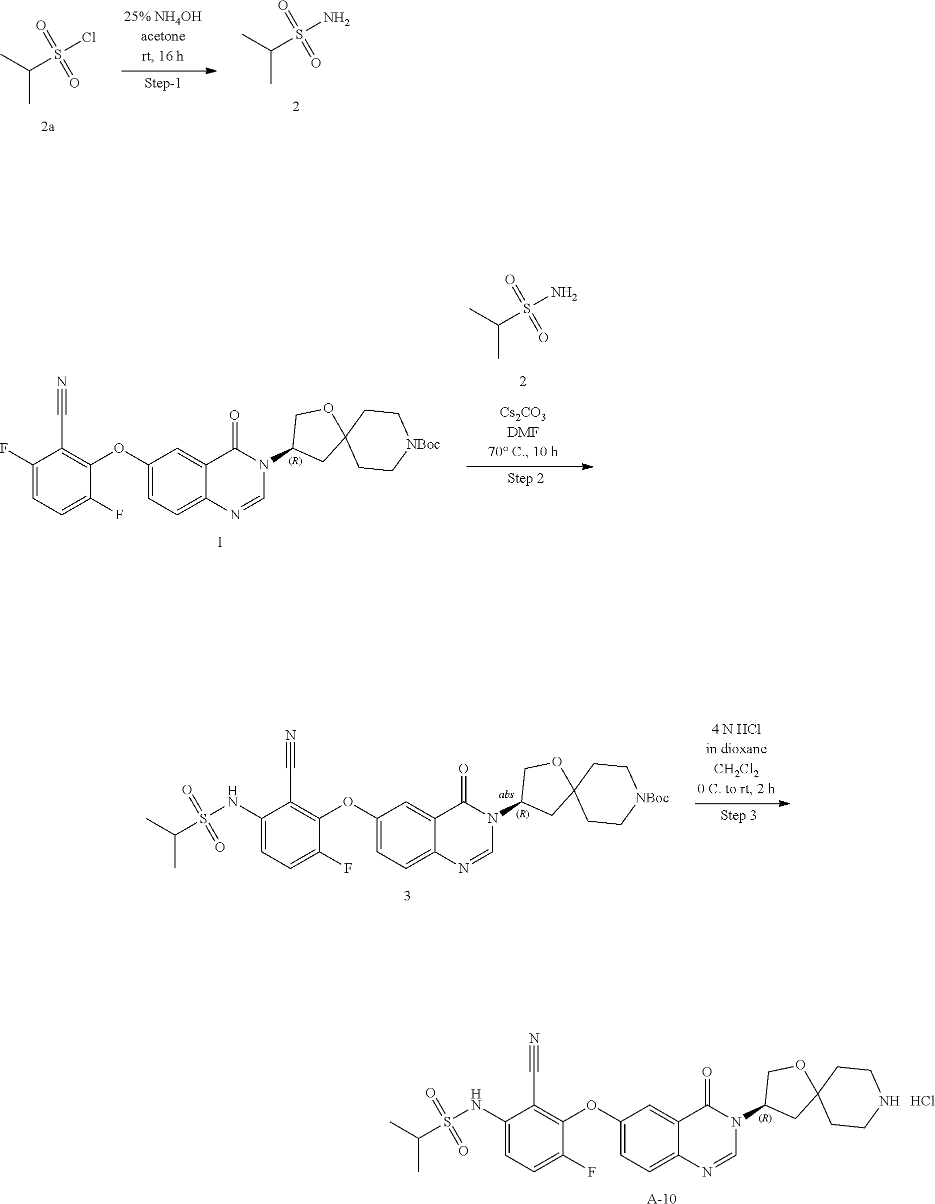

### Step 1

Into a 250 mL sealed tube containing a well-stirred solution of propane-2-sulfonyl chloride (2a, 7 g, 49.09 mmol, 5.51 mL) in acetone (35 mL) was added ammonium hydroxide, 25% NH₃ in water (1.80 mol, 70 mL) slowly at 0° C. The reaction mixture was stirred at room temperature for 16 hours. After completion of reaction, the reaction mixture was concentrated under reduced pressure to get crude residue, which was diluted with water (100 mL) and extracted with ethyl acetate (3×200 mL). Combined organic layer was dried over anhydrous Na₂SO₄, filtered and concentrated under reduced pressure to afford crude residue which was purified by flash silica-gel (100-200 mesh) column chromatography using 0-100% ethyl acetate/hexane as an eluent to afford propane-2-sulfonamide (2, 2.8 g, 20.46 mmol, 42% yield) as an off-white solid. ¹H NMR (400 MHz, DMSO-d₆): δ 6.63 (bs, 2H), 3.06-2.99 (m, 1H), 1.23 (d, J=6.8 Hz, 6H).

### Step 2

Into a 20 mL pressure vial containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (1, 500 mg, 928.43 mol) and propane-2-sulfonamide (2, 228.72 mg, 1.86 mmol) in N,N-dimethylformamide (5 mL) was added Cs₂CO₃ (756.25 mg, 2.32 mmol) at room temperature under nitrogen atmosphere. The resulting reaction mixture was stirred at 70° C. for 10 hours. After completion of reaction, the reaction mixture was diluted with water (50 mL) and extracted with ethyl acetate (2×100 mL). The combined organic layer was dried over anhydrous Na₂SO₄, filtered and concentrated under reduced pressure to get a crude residue, which was purified by flash silica gel (100-200 mesh) column chromatography using 0-100% ethyl acetate/hexane as an eluent to afford tert-butyl(3R)-3-[6-[2-cyano-6-fluoro-3-(isopropylsulfonylamino)phenoxy]-4-oxo-quinazolin-3-yl]-1-oxa-8-azaspiro[4.5]decane-8-carboxylate (3, 400 mg, 598.15 mol, 64% yield) as a pale brown gummy liquid. UPLC-MS (ES+): m/z 586.0 [M-^{t}Bu+H]⁺.

### Step 3

Into a 100 mL single-neck round-bottom flask containing a well-stirred solution of tert-butyl(3R)-3-[6-[2-cyano-6-fluoro-3-(isopropylsulfonylamino)phenoxy]-4-oxo-quinazolin-3-yl]-1-oxa-8-azaspiro[4.5]decane-8-carboxylate (3,400 mg, 623.34 mol) in dichloromethane (10 mL) was added hydrogen chloride solution (4 M in 1,4-dioxane, 3.12 mL) at 0° C. under nitrogen atmosphere. Resulting reaction mixture was stirred at room temperature for 2 hours. After completion of reaction, reaction mixture was concentrated under reduced pressure to afford a crude N-[2-cyano-4-fluoro-3-[3-[(3R)-1-oxa-8-azaspiro[4.5]decan-3-yl]-4-oxo-quinazolin-6-yl]oxy-phenyl]propane-2-sulfonamide (A-10, 360 mg, 598.43 mol, 96% yield, HCl salt) as an off-white solid. UPLC-MS (ES+): m/z 542.2 [M+H]⁺.

### Synthesis A11: Synthesis of N-[2-cyano-4-fluoro-3-[4-oxo-3-[rac-(3R)-1-oxa-8-azaspiro[4.5]decan-3-yl]quinazolin-6-yl]oxy-phenyl]cyclopentanesulfonamide (A-11)

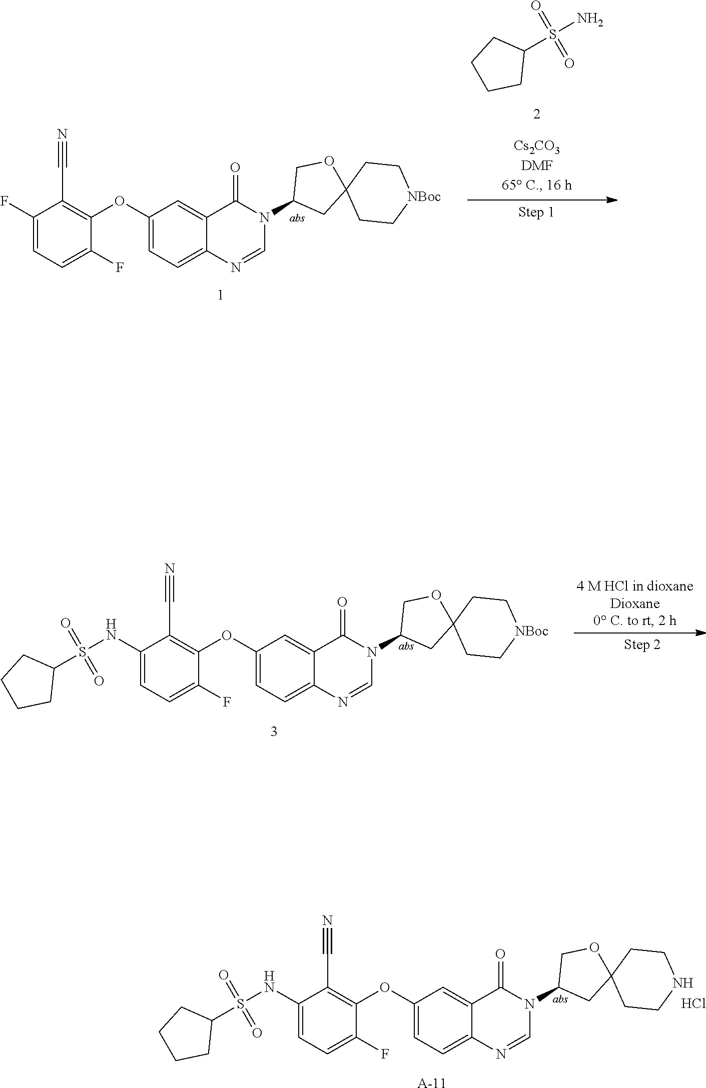

### Step 1

Into a 20 mL sealed-vial containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (1, 500 mg, 928.43 mol) in N,N-dimethylformamide (5 mL) was added Cs₂CO₃ (302.50 mg, 928.43 mol) followed by cyclopentane sulfonamide (2, 253.98 mg, 1.02 mmol) at room temperature. The reaction mixture was stirred at 65° C. for 16 hours. The reaction mixture was allowed to cool to room temperature and diluted with ethyl acetate (40 mL). The mixture was filtered on Celite pad and pad was washed with ethyl acetate (30 mL). Filtrate was concentrated under reduced pressure to afford a crude residue which was purified by flash silica-gel (230-400 mesh, 50 g) column using 0-100% ethyl acetate/hexane as an eluent to afford tert-butyl(3R)-3-[6-[2-cyano-3-(cyclopentylsulfonylamino)-6-fluoro-phenoxy]-4-oxo-quinazolin-3-yl]-1-oxa-8-azaspiro[4.5]decane-8-carboxylate (3, 500 mg, 582.18 mol, 63% yield) as a yellow colored solid. UPLC-MS (ES+): m/z 668.4 [M+H]⁺.

### Step 2

Into a 100 mL single-neck round-bottom flask containing a well-stirred solution of tert-butyl-(3R)-3-[6-[2-cyano-3-(cyclopentylsulfonylamino)-6-fluoro-phenoxy]-4-oxo-quinazolin-3-yl]-1-oxa-8-azaspiro[4.5]decane-8-carboxylate (3, 350 mg, 524.15 mol) in anhydrous dichloromethane (4 mL) was added 4M HCl in dioxane (4 M, 2.62 mL) at 0° C. under nitrogen atmosphere. The resulting suspension was stirred at room temperature for 2 hours. The reaction mixture was concentrated under reduced pressure get a crude residue which was triturated with methyl tert-butyl ether (2×4 mL) to afford N-[2-cyano-4-fluoro-3-[4-oxo-3-[rac-(3R)-1-oxa-8-azaspiro[4.5]decan-3-yl]quinazolin-6-yl]oxy-phenyl]cyclopentanesulfonamide (A-11, 300 mg, 486.48 mol, 93% yield, HCl salt) as an off-white solid. UPLC-MS (ES+): m/z 568.2 [M+H]⁺.

### Synthesis A12: Synthesis of (3R)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-12)

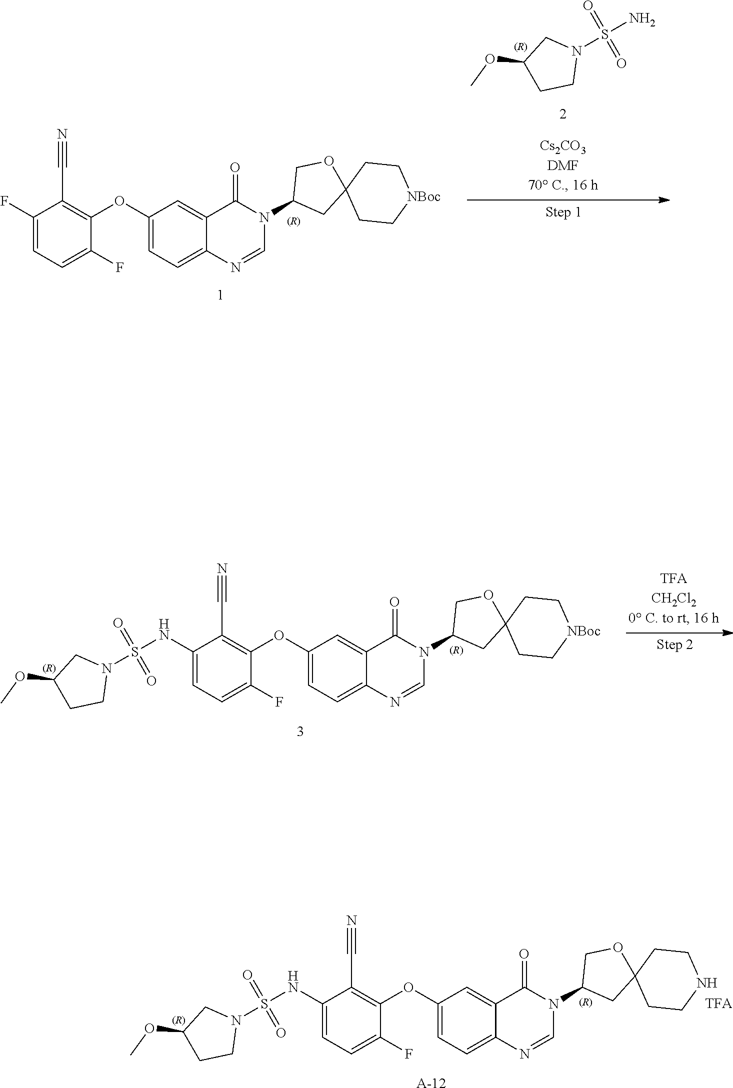

### Step 1

Into a 20 mL pressure vial containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (1, 600.00 mg, 1.11 mmol) in N,N-dimethylformamide (8 mL) were added (3R)-3-methoxypyrrolidine-1-sulfonamide (2, 602.36 mg, 3.34 mmol) and Cs₂CO₃ (1.45 g, 4.46 mmol) at room temperature under nitrogen atmosphere. The reaction mixture was stirred at 70° C. for 16 hours. After completion of the reaction, the reaction mixture was diluted with water (30 mL) and filtered through Buchner funnel to remove the fluorescent impurity. The filtrate was extracted with ethyl acetate (3×30 mL) and the combined organic layers were washed with cold water (3×30 mL), dried over anhydrous Na₂SO₄, filtered and concentrated under reduced pressure to afford tert-butyl(3R)-3-[6-[2-cyano-6-fluoro-3-[[(3R)-3-methoxypyrrolidin-1-yl]sulfonylamino]phenoxy]-4-oxo-quinazolin-3-yl]-1-oxa-8-azaspiro[4.5]decane-8-carboxylate (3, 478 mg, 619.29 mol, 56% yield) as a brown gum. UPLC-MS (ES−): m/z 697.2 [M−H]⁻.

### Step 2

Into a 50 mL single-neck round-bottom flask containing a well-stirred solution of tert-butyl(3R)-3-[6-[2-cyano-6-fluoro-3-[[(3R)-3-methoxypyrrolidin-1-yl]sulfonylamino]phenoxy]-4-oxo-quinazolin-3-yl]-1-oxa-8-azaspiro[4.5]decane-8-carboxylate (3, 478 mg, 619.29 mol) in dichloromethane (12 mL) was added trifluoroacetic acid (6.19 mmol, 473.74 L) slowly at 0° C. under nitrogen atmosphere. The resulting solution was stirred at room temperature for 16 hours. After completion of the reaction, the reaction mixture was concentrated under reduced pressure and the obtained residue was washed with methyl tert-butyl ether to remove the non-polar impurity to afford (3R)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-12, 550 mg, 609.30 mol, 98% yield, TFA salt) as a brown gum. UPLC-MS (ES+): m/z 599.3 [M+H]⁺.

### Synthesis A13: Synthesis of (3S)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-13)

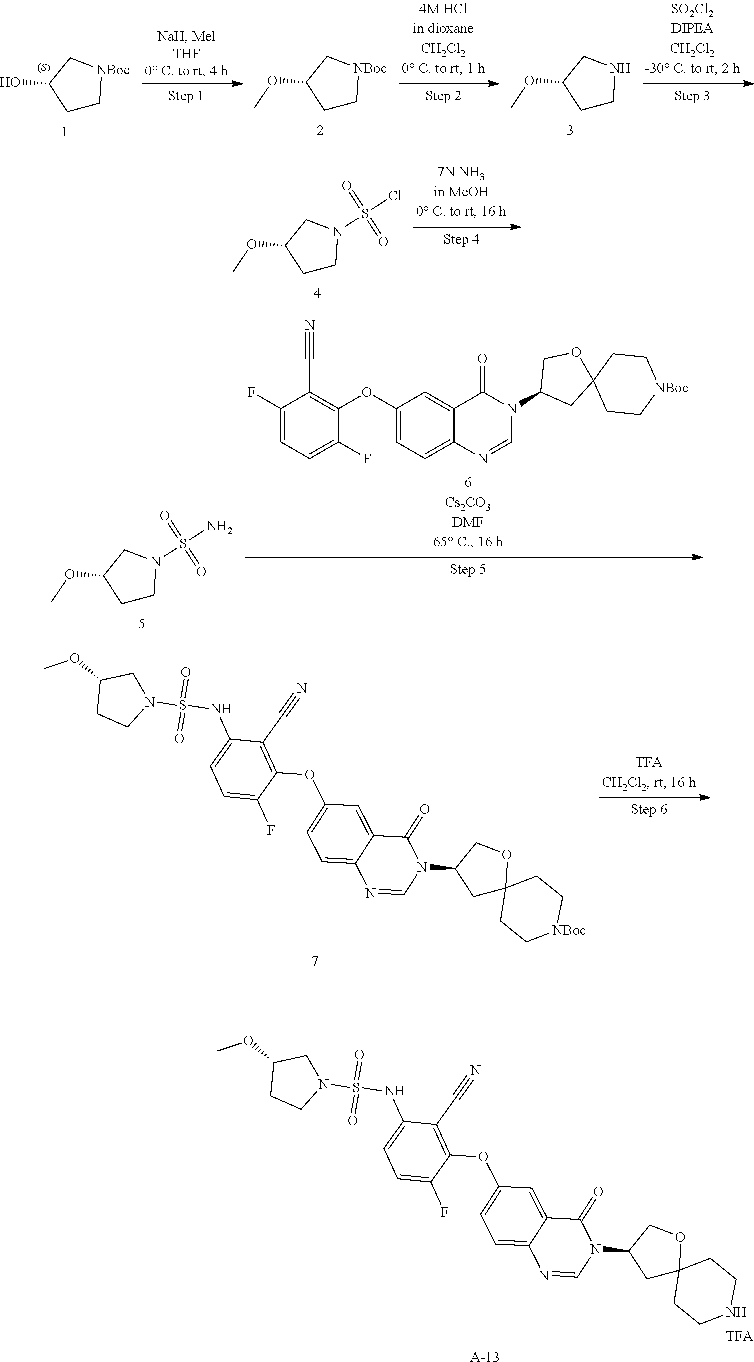

### Step 1

Into a 500 mL two-neck round-bottom flask containing a well-stirred solution of sodium hydride (4.63 g, 106.82 mmol, 60% purity) in THF (180 mL) was added tert-butyl (3S)-3-hydroxypyrrolidine-1-carboxylate (1, 10 g, 53.41 mmol) at 0° C. and resulting solution was stirred for 1 hour at the same temperature. After 1 h, iodomethane (80.11 mmol, 4.99 mL) was added to the above mixture at 0° C. Reaction mixture was stirred at room temperature for 4 hours. After completion of the reaction, the reaction mixture was quenched with crushed ice slowly and extracted with ethyl acetate (3×100 mL). The combined organic layer was dried over anhydrous Na₂SO₄, filtered, and concentrated under reduced pressure to afford tert-butyl (3S)-3-methoxypyrrolidine-1-carboxylate (2, 10 g, 49.69 mmol, 93% yield) as a light brown gummy liquid. ¹H NMR (400 MHz, DMSO-d₆): δ 3.90-3.89 (m, 1H), 3.30-3.17 (m, 7H), 1.91-1.85 (m, 2H), 1.40 (s, 9H).

### Step 2

Into a 500 mL two-neck round-bottom flask containing a well-stirred solution of tert-butyl (3S)-3-methoxypyrrolidine-1-carboxylate (2, 23 g, 114.28 mmol) in dichloromethane (70 mL) was added hydrogen chloride solution (4.0 M in 1,4-dioxane) (1.14 mol, 285.7 mL) at 0° C. Reaction was stirred at room temperature for 1 hour. After completion of the reaction, the reaction mixture was concentrated under reduced pressure to afford crude (3S)-3-methoxypyrrolidine (3, 14.3 g, 103.92 mmol, 91% yield, HCl salt) as a dark green solid. ¹H NMR (400 MHz, DMSO-d₆): δ 9.59 (bs, 1H), 9.28 (bs, 1H), 4.08-4.06 (m, 1H), 3.22-3.05 (m, 7H), 2.06-1.99 (m, 1H), 1.94-1.83 (m, 1H).

### Step 3

Into a 50 mL, two-neck round-bottom flask containing a well-stirred solution of (3S)-3-methoxypyrrolidine (3, 2 g, 14.53 mmol, HCl salt) in dichloromethane (6.60 mL) was added N,N-diisopropylethylamine (23.25 mmol, 4.05 mL) and sulfuryl chloride (37.79 mmol, 3.06 mL) at −30° C. under nitrogen atmosphere. Reaction was stirred at room temperature for 2 hours. After completion of the reaction, the reaction mixture was poured slowly into ice and extracted with dichloromethane (3×40 mL). The combined organic layer was dried over Na₂SO₄, filtered, and concentrated under reduced pressure to afford a crude (3S)-3-methoxypyrrolidine-1-sulfonyl chloride (4, 2.65 g, 13.27 mmol, 91% yield) as a yellow liquid. ¹H NMR (400 MHz, DMSO-d₆): δ 4.08-4.06 (m, 1H), 3.22-3.05 (m, 7H), 2.06-1.99 (m, 1H), 1.94-1.83 (m, 1H).

### Step 4

Into a 100 mL sealed tube containing a well-stirred solution of (3S)-3-methoxypyrrolidine-1-sulfonyl chloride (4, 2.6 g, 13.02 mmol) in methanol (11.74 mL) was added ammonia (7 M in methanol) (58.60 mmol, 19 mL) dropwise at 0° C. The reaction mixture was stirred at room temperature for 16 hours. After completion of the reaction, the reaction mixture was concentrated, and obtained crude material was dissolved in ethyl acetate (20 mL) and washed with water (2×20 mL). The combined organic layer was washed with saturated NaCl solution (40 mL), dried over anhydrous Na₂SO₄, filtered and concentrated under reduced pressure to afford a crude (3S)-3-methoxypyrrolidine-1-sulfonamide (5, 1.15 g, 6.38 mmol, 49% yield) as a brown solid. ¹H NMR (400 MHz, DMSO-d₆): δ 6.71 (s, 2H), 3.98-3.94 (m, 1H), 3.27-3.22 (m, 4H), 3.18-3.07 (m, 3H), 1.97-1.84 (m, 2H).

### Step 5

Into a 30 mL pressure vial containing a well-stirred solution of (3S)-3-methoxypyrrolidine-1-sulfonamide (5, 401.58 mg, 2.23 mmol) and tert-butyl(3R)-3-[6-(2-cyano-3,6-difluoro-phenoxy)-4-oxo-quinazolin-3-yl]-1-oxa-8-azaspiro[4.5]decane-8-carboxylate (6, 400 mg, 742.75 mol) in N,N-dimethylformamide (8 mL) was added Cs₂CO₃ (968.00 mg, 2.97 mmol) at room temperature under nitrogen atmosphere. Reaction was stirred at 65° C. for 16 hours. After completion of the reaction, the reaction mixture was diluted with ice-cold water (20 mL) and filtered through a celite pad and filtrate was extracted with ethyl acetate (3×40 mL). Combined organic extracts were dried over anhydrous Na₂SO₄, filtered and concentrated under reduced pressure to afford a crude tert-butyl(3R)-3-[6-[2-cyano-6-fluoro-3-[[(3S)-3-methoxypyrrolidin-1-yl]sulfonylamino]phenoxy]-4-oxo-quinazolin-3-yl]-1-oxa-8-azaspiro[4.5]decane-8-carboxylate (7, 480 mg, 615.49 mol, 83% yield) as a brown viscous liquid. UPLC-MS (ES+): m/z 699.4 [M+H]⁺.

### Step 6

Into a 50 mL single-neck round-bottom flask containing a well-stirred solution of tert-butyl(3R)-3-[6-[2-cyano-6-fluoro-3-[[(3S)-3-methoxypyrrolidin-1-yl]sulfonylamino]phenoxy]-4-oxo-quinazolin-3-yl]-1-oxa-8-azaspiro[4.5]decane-8-carboxylate (7, 480 mg, 686.93 mol) in dichloromethane (8 mL) was added trifluoroacetic acid (17.17 mmol, 1.31 mL) at 0° C. under nitrogen atmosphere. Reaction was stirred at room temperature for 16 hours. After completion of the reaction, the reaction mixture was concentrated under reduced pressure and triturated with methyl tert-butyl ether to afford crude (3S)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-13, 450 mg, 582.68 mol, 85% yield, TFA salt) as a brown sticky liquid. UPLC-MS (ES+): m/z 599.3 [M+H]⁺.

### Synthesis A14: Synthesis of (3R)-N-[2-cyano-4-fluoro-3-[3-[(3R)-1-oxa-8-azaspiro[4.5]decan-3-yl]-4-oxo-quinazolin-6-yl]oxy-phenyl]-3-fluoro-pyrrolidine-1-sulfonamide (A-14)

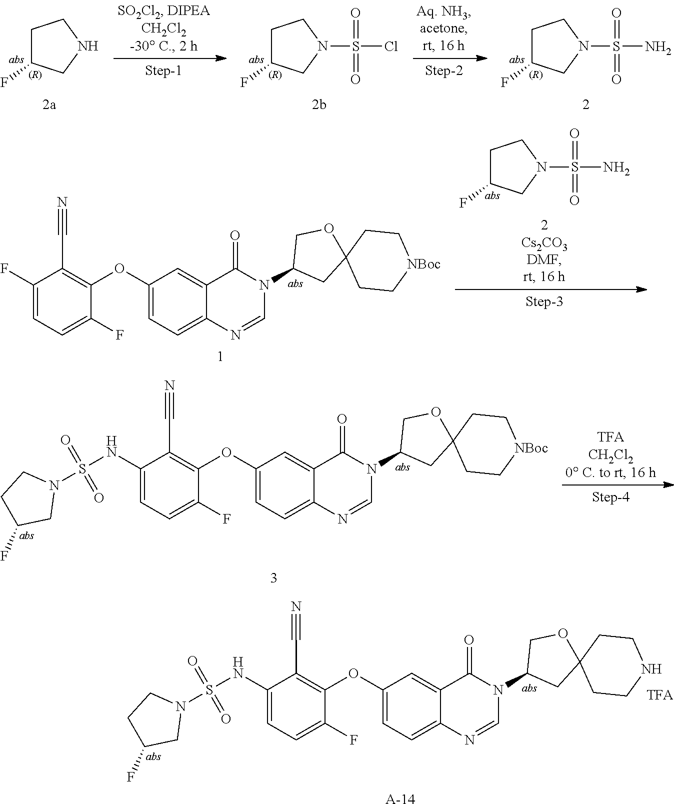

### Step 1

Into a 50 mL two-neck round-bottom flask containing a well-stirred solution of (3R)-3-fluoropyrrolidine (2a, 3.0 g, 23.89 mmol, HCl salt) in dichloromethane (30 mL) was added N,N-diisopropylethylamine (38.23 mmol, 6.66 mL) at −30° C. followed by sulfuryl chloride (59.73 mmol, 4.84 mL) addition in drop wise and stirred at room temperature for 2 hours. After completion of the reaction, the reaction mixture was quenched with water (30 mL) and extracted with dichloromethane (2×30 mL). The combined organic layer was washed with 1.5 N HCl (20 mL), dried over anhydrous Na₂SO₄, filtered and concentrated under reduced pressure to afford crude (3R)-3-fluoropyrrolidine-1-sulfonyl chloride (2b, 3.6 g, 19.19 mmol, 80% yield) as an off-white solid. ¹H NMR (400 MHz, DMSO-d₆): δ 5.53-5.37 (m, 1H), 3.76-3.67 (m, 4H), 2.32-2.29 (m, 2H).

### Step 2

Into a 250 mL two-neck round bottom flask containing a well stirred solution of (3R)-3-fluoropyrrolidine-1-sulfonyl chloride (2b, 3.6 g, 19.19 mmol) in acetone (50 mL) was added aqueous ammonia (191.88 mmol, 29.89 mL, 25% purity) at 0° C. and reaction mixture was allowed to warm to room temperature and stirred for 16 hours. After completion of the reaction, the reaction mixture was quenched by 1.5 N HCl at 0° C. and extracted with ethyl acetate (2×200 mL). Combined organic layer was dried over anhydrous Na₂SO₄ and concentrated under reduced pressure to get a crude residue which was purified by flash column chromatography (100 g, 60-120 silica-gel mesh) using 0-100% ethyl acetate in hexane as an eluent to afford (3R)-3-fluoropyrrolidine-1-sulfonamide (2, 1.5 g, 8.73 mmol, 46% yield) as a brown solid. UPLC-MS (ES+): m/z 169.0 [M+H]⁺.

### Step 3

Into a 25 mL single-neck round bottom flask containing a well-stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (1, 500.00 mg, 928.43 mol) and (3R)-3-fluoropyrrolidine-1-sulfonamide (2, 265.46 mg, 1.58 mmol) in N,N-dimethylformamide (5 mL) was added Cs₂CO₃ (1.06 g, 3.25 mmol) at room temperature under nitrogen atmosphere. The resulting mixture was stirred at room temperature for 16 hours. The reaction mixture was diluted with water (20 mL) and extracted with ethyl acetate (2×20 mL). The combined organic layer was washed with water (30 mL), saturated NaCl (30 mL), dried over anhydrous sodium sulphate and concentrated under reduced pressure to get a crude residue which was purified by the flash column chromatography (25 g, 230-400 silica-gel mesh) using 0-100% ethyl acetate in hexane as an eluent to afford tert-butyl(3R)-3-[6-[2-cyano-6-fluoro-3-[[(3R)-3-fluoropyrrolidin-1-yl]sulfonylamino]phenoxy]-4-oxo-quinazolin-3-yl]-1-oxa-8-azaspiro[4.5]decane-8-carboxylate (3, 0.220 g, 304.34 mol, 33% yield) as an off-white solid. UPLC-MS (ES−): m/z 685.2 [M−H]⁻.

### Step 4

Into a 25 mL single-neck round bottom flask containing a well-stirred solution of tert-butyl(3R)-3-[6-[2-cyano-6-fluoro-3-[[(3R)-3-fluoropyrrolidin-1-yl]sulfonylamino]phenoxy]-4-oxo-quinazolin-3-yl]-1-oxa-8-azaspiro[4.5]decane-8-carboxylate (3, 220.00 mg, 320.36 mol) in dichloromethane (5.0 mL) was added trifluoroacetic acid (1.60 mmol, 122.58 μL) at 0° C. under nitrogen atmosphere. The resulting mixture was stirred at room temperature for 16 hours. After completion of the reaction, the reaction mixture was concentrated under reduced pressure and triturated with methyl tert-butyl ether (10 mL) to afford (3R)-N-[2-cyano-4-fluoro-3-[3-[(3R)-1-oxa-8-azaspiro[4.5]decan-3-yl]-4-oxo-quinazolin-6-yl]oxy-phenyl]-3-fluoro-pyrrolidine-1-sulfonamide (A-14, 0.160 g, 216.95 μmol, 68% yield, TFA salt) as an off-white solid. UPLC-MS (ES+): m/z 587.2 [M+H]⁺.

### Synthesis A15: Synthesis of N-[2-cyano-4-fluoro-3-[3-[(3R)-1-oxa-8-azaspiro[4.5]decan-3-yl]-4-oxo-quinazolin-6-yl]oxy-phenyl]-2-oxa-6-azaspiro[3.3]heptane-6-sulfonamide (A-15)

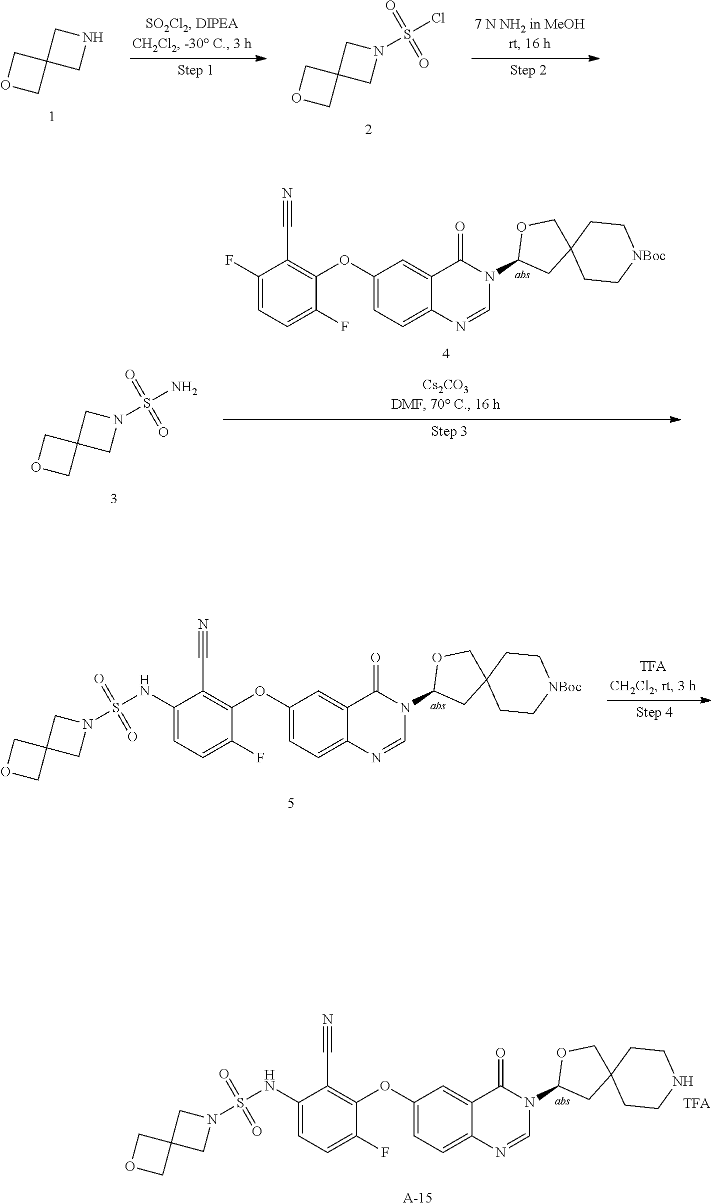

### Step 1

Into a 100 mL two-neck round bottom flask containing a well stirred suspension of 2-oxa-6-azaspiro[3.3]heptane (1, 1 g, 10.09 mmol) in dichloromethane (20 mL) was added N,N-diisopropylethylamine (40.35 mmol, 7.03 mL) at room temperature and stirred for 10 min. sulfuryl chloride (18.16 mmol, 1.47 mL) was added drop wise at −30° C. under nitrogen atmosphere and the resulting mixture was stirred for 3 hours. The reaction mixture was quenched with water (50 mL) and extracted with dichloromethane (2×30 mL). The combined organic layer was washed with 1.5N HCl (30 mL), saturated NaCl solution (30 mL), dried over anhydrous sodium sulphate, filtered and concentrated under reduced pressure to afford 2-oxa-6-azaspiro[3.3]heptane-6-sulfonyl chloride (2, 1.1 g, 2.78 mmol, 28% yield) as a brown gum. The obtained crude compound was taken to next step. ¹H NMR (400 MHz, DMSO-d₆): δ 4.67 (s, 4H), 4.37 (s, 4H).

### Step 2

Into a 50 mL single neck round bottom flask containing a well stirred solution of 2-oxa-6-azaspiro[3.3]heptane-6-sulfonyl chloride (2, 1.1 g, 2.78 mmol) in methanol (10 mL) was added 7M Ammonia in methanol (7 M, 10 mL) dropwise at 0° C. under nitrogen atmosphere. The resulting mixture was stirred at room temperature for 16 hours. After completion of the reaction, the reaction mixture was concentrated under reduced pressure to get a crude residue, which was purified by flash column chromatography (50 g, neutral alumina) and eluting with 0-10% methanol in dichloromethane to afford 2-oxa-6-azaspiro[3.3]heptane-6-sulfonamide (3, 500 mg, 2.70 mmol, 97% yield) as a brown color solid. ELSD-MS (ES−): m/z 177.0 [M−H]⁻.

### Step 3

Into a 30 mL glass pressure vial containing a well stirred solution of tert-butyl(3R)-3-[6-(2-cyano-3,6-difluoro-phenoxy)-4-oxo-quinazolin-3-yl]-1-oxa-8-azaspiro[4.5]decane-8-carboxylate (4, 300 mg, 540.35 mol) in N,N-dimethylformamide (3 mL) were added Cs₂CO₃ (528.17 mg, 1.62 mmol) and 2-oxa-6-azaspiro[3.3]heptane-6-sulfonamide (3, 288.89 mg, 1.62 mmol) at room temperature under nitrogen atmosphere. The resulting mixture was stirred at 70° C. for 16 hours. After completion of the reaction, the reaction mixture was cooled to room temperature, diluted with ice cold water (10 mL) and stirred for 10 min. The precipitated solid was filtered, and the filtrate was extracted with ethyl acetate (3×30 mL). The combined organic layers were washed with ice cold water (2×50 mL), saturated NaCl solution (50 mL) and concentrated under reduced pressure to afford tert-butyl(3R)-3-[6-[2-cyano-6-fluoro-3-(2-oxa-6-azaspiro[3.3]heptan-6-ylsulfonylamino)phenoxy]-4-oxo-quinazolin-3-yl]-1-oxa-8-azaspiro[4.5]decane-8-carboxylate (5, 180 mg, 202.21 mol, 37% yield) as an off-white solid. UPLC-MS (ES+): m/z 697.2 [M+H]⁺.

### Step 4

Into a 50 mL single neck round bottom flask containing a well stirred solution of tert-butyl(3R)-3-[6-[2-cyano-6-fluoro-3-(2-oxa-6-azaspiro[3.3]heptan-6-ylsulfonylamino)phenoxy]-4-oxo-quinazolin-3-yl]-1-oxa-8-azaspiro[4.5]decane-8-carboxylate (5, 180 mg, 202.21 mol) in dichloromethane (10 mL) was added trifluoroacetic acid (6.53 mmol, 0.5 mL) at 0° C. under nitrogen atmosphere. The reaction mixture stirred at room temperature for 3 hours. After completion of the reaction, the reaction mixture was concentrated under reduced pressure to get a residue, which was triturated with methyl tert-butyl ether (3 mL) and dried under vacuum to afford N-[2-cyano-4-fluoro-3-[3-[(3R)-1-oxa-8-azaspiro[4.5]decan-3-yl]-4-oxo-quinazolin-6-yl]oxy-phenyl]-2-oxa-6-azaspiro[3.3]heptane-6-sulfonamide (A-15, 160 mg, 176.58 mol, 87% yield, TFA salt) as a pale yellow gummy-solid. UPLC-MS (ESI): m/z 597.2 [M+H]⁺.

### Synthesis A16: Synthesis of 6-[2-cyano-3-[[ethyl(methyl)sulfamoyl]amino]-6-fluoro-phenoxyl-4-oxo-3-[(3R)-8-oxo-1-oxaspiro[4.5]decan-3-yl]quinazoline (A-16)

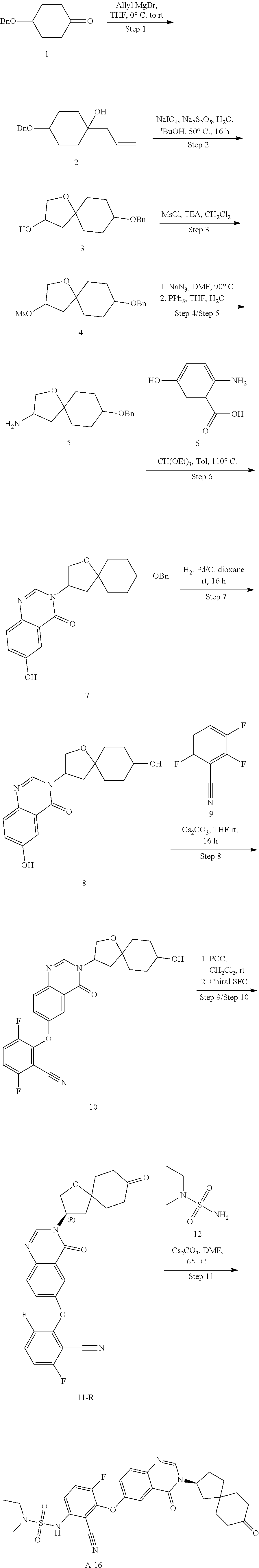

### Step 1

Into a 500 mL two neck round bottom flask containing a well-stirred solution of 4-benzyloxycyclohexanone (1, 10 g, 48.96 mmol) in anhydrous tetrahydrofuran (150 mL) was added allyl magnesium bromide (1 M, 122.39 mL) at 0-5° C. under nitrogen atmosphere. The resulting mixture was stirred at ambient temperature for 6 hours. After completion of the reaction, saturated ammonium chloride (100 mL) was added to the reaction mixture and extracted with ethyl acetate (3×100 mL). The combined organic layers were dried over anhydrous Na₂SO₄, filtered and the filtrate was concentrated under reduced pressure to get crude residue. The crude compound was purified by flash silica-gel column chromatography eluting at 40-50% of ethyl acetate in petroleum ether to afford 1-allyl-4-benzyloxy-cyclohexanol (2, 3.7 g, 12.85 mmol, 26% yield) as a colourless oily liquid. ¹H NMR (400 MHz, DMSO-d₆): δ 7.26-7.31 (m, 5H), 5.82-5.91 (m, 1H), 4.96-5.02 (m, 1H), 4.48 (s, 1H), 4.43 (d, J=8.80 Hz, 1H), 4.11 (s, 1H), 3.20-3.35 (m, 1H), 2.55-2.30 (m, 2H), 2.10 (d, J=9.60 Hz, 1H), 1.45-1.81 (m, 6H), 1.15-1.41 (m, 2H).

### Step 2

Into a 250 mL single neck round bottom flask containing a well-stirred solution of 1-allyl-4-benzyloxy-cyclohexanol (2, 3.70 g, 15.02 mmol) in a mixture of tert-butanol (40 mL) and water (20 mL) was added sodium metaperiodate (3.60 g, 16.83 mmol) at 0-5° C. To this reaction mixture, sodium metabisulfite (3.20 g, 16.83 mmol, 2.16 mL) in water (10 mL) was added at the same temperature. The resulting mixture was heated to 50° C. for 16 hours. After completion of the reaction, the reaction mixture was cooled to room temperature and poured into water (100 mL) and extracted with ethyl acetate (3×100 mL). The combined organic phases were dried over anhydrous Na₂SO₄, filtered and the filtrate was concentrated under reduced pressure to get a crude residue. The crude product purified by flash silica-gel column chromatography eluting with 70-80% of ethyl acetate in petroleum ether to afford 8-benzyloxy-1-oxaspiro[4.5]decan-3-ol (3, 1.10 g, 4.15 mmol, 28% yield) as a brownish gummy liquid. UPLC-MS (ES+): m/z 263.4 [M+H]⁺.

### Step 3

Into a 50 mL single neck round bottom flask containing a well-stirred solution of 8-benzyloxy-1-oxaspiro[4.5]decan-3-ol (3, 1.10 g, 4.19 mmol) in anhydrous dichloromethane (15 mL) was added triethylamine (14.35 mmol, 2.0 mL) at 0-5° C., followed by the addition of methane sulfonyl chloride (6.46 mmol, 0.500 mL) at same temperature. The resulting mixture was stirred at ambient temperature for 16 hours. After completion of the reaction, water (50 mL) was added to the reaction mixture and extracted with dichloromethane (2×100 mL). The combined organic layers were dried over anhydrous Na₂SO₄, filtered and the filtrate was concentrated under reduced pressure to get crude residue. The crude compound was purified by flash silica-gel column chromatography eluting with 40-50% ethyl acetate in petroleum ether to afford (8-benzyloxy-1-oxaspiro[4.5]decan-3-yl) methane sulfonate (4, 1.0 g, 2.35 mmol, 56% yield) as yellowish gummy liquid. UPLC-MS (ES+): m/z 341.4 [M+H]⁺.

### Step 4

Into a 50 mL single-neck round-bottom flask containing a well-stirred solution of (8-benzyloxy-1-oxaspiro[4.5]decan-3-yl) methane sulfonate (4, 1.0 g, 2.94 mmol) in anhydrous N,N-dimethylformamide (10 mL) was added sodium azide (300 mg, 4.61 mmol, 162.16 μL) at ambient temperature under nitrogen atmosphere. The resulting mixture was heated to 80° C. for 16 hours. After completion of the reaction, water (50 ml) was added to the reaction mixture and extracted with ethyl acetate (2×100 mL). The combined organic layers were dried over Na₂SO₄, filtered, and concentrated under reduced pressure to get crude residue. The crude residue was purified by flash silica-gel column chromatography eluted with 70-80% of ethyl acetate in petroleum ether to afford 3-azido-8-benzyloxy-1-oxaspiro[4.5]decane (780 mg, 2.23 mmol, 76% yield) as a brownish gummy liquid. UPLC-MS (ES+): m/z 288.4 [M+H]⁺.

### Step 5

Into a 50 mL single-neck round-bottom flask containing a well-stirred solution of 3-azido-8-benzyloxy-1-oxaspiro[4.5]decane (780 mg, 2.71 mmol) in a mixture of tetrahydrofuran(10 mL) and water (1.0 mL) was added triphenyl phosphine (1.10 g, 4.19 mmol) at ambient temperature. The resulting mixture was heated to 80° C. for 3 hours. After completion of the reaction, water (50 ml) was added to the reaction mixture and extracted with ethyl acetate (3×100 mL). The combined organic layers were dried over Na₂SO₄, filtered, and concentrated under reduced pressure to get crude residue. The crude residue was purified by flash silica-gel column chromatography eluted with 0-20% methanol in dichloromethane to afford 8-benzyloxy-1-oxaspiro[4.5]decan-3-amine (5, 510 mg, 1.85 mmol, 68% yield) as a brownish gummy liquid. LC-MS(ES+): m/z 262.4 [M+H]⁺.

### Step 6

Into a 50 mL sealed-tube containing a well-stirred solution of 8-benzyloxy-1-oxaspiro[4.5]decan-3-amine (5, 510 mg, 1.95 mmol) in a mixture of toluene (10.0 mL) and tetrahydrofuran (3.0 mL) were added 2-amino-5-hydroxy-benzoic acid (6, 370 mg, 2.42 mmol) and triethyl orthoformate (5.41 mmol, 0.900 mL) at ambient temperature under nitrogen atmosphere. The resulting mixture was heated to 110° C. for 16 hours. After completion, the reaction mixture was cooled to room temperature and poured into water (50 mL) and extracted with ethyl acetate (2×100 mL). The combined organic phases were dried over anhydrous Na₂SO₄, filtered and the filtrate was concentrated under reduced pressure. The crude residue was purified by flash silica-gel column chromatography eluting with 5-10% of methanol in dichloromethane to afford 3-(8-benzyloxy-1-oxaspiro[4.5]decan-3-yl)-6-hydroxy-quinazolin-4-one (7, 560 mg, 1.30 mmol, 66% yield) as a pale brown solid. UPLC-MS (ES+): m/z 407.7 [M+H]⁺.

### Step 7

Into a 50 mL single-neck round-bottom flask containing a well-stirred solution of 3-(8-benzyloxy-1-oxaspiro[4.5]decan-3-yl)-6-hydroxy-quinazolin-4-one (7, 560 mg, 1.38 mmol) in anhydrous 1,4-dioxane (10 mL) was added acetic acid (1.75 mmol, 0.100 mL) and charged 10% palladium on carbon (150 mg, 140.95 mol, 10% purity), saturated with hydrogen by bubbling hydrogen gas through for 10 minutes and subjected to hydrogenation (1 atm) at ambient temperature for 16 hours. After completion, the reaction mixture was purged with nitrogen, catalyst was removed by filtration through celite pad. The filtrate was concentrated under reduced pressure. The crude product was purified by flash silica-gel column chromatography eluting with 10-15% of methanol in dichloromethane to afford 6-hydroxy-3-(8-hydroxy-1-oxaspiro[4.5]decan-3-yl)quinazolin-4-one (8, 340 mg, 913.54 mol, 66% yield) as a pale yellow solid. UPLC-MS (ES+): m/z 317.2 [M+H]⁺.

### Step 8

Into a 25 mL sealed tube containing a well-stirred solution of 6-hydroxy-3-(8-hydroxy-1-oxaspiro[4.5]decan-3-yl)quinazolin-4-one (8, 340 mg, 1.07 mmol) in anhydrous tetrahydrofuran (10 mL) were added 2,3,6-trifluorobenzonitrile (1.66 mmol, 191.18 μL) and Cs₂CO₃ (1.10 g, 3.38 mmol) at ambient temperature under nitrogen atmosphere. The resulting mixture was stirred at ambient temperature for 16 hours. After completion of the reaction, water (50 ml) was added to the reaction mixture and extracted with ethyl acetate (2×100 mL). The combined organic layers were dried over Na₂SO₄, filtered, and concentrated under reduced pressure to get crude residue. The crude residue was purified by flash silica-gel column chromatography eluting with 80-90% ethyl acetate in petroleum ether to afford 3,6-difluoro-2-[3-(8-hydroxy-1-oxaspiro[4.5]decan-3-yl)-4-oxo-quinazolin-6-yl]oxy-benzonitrile (10, 380 mg, 687.20 mol, 64% yield) as yellowish gummy liquid. UPLC-MS (ES+): m/z 454.1 [M+H]⁺.

### Step 9/Step 10

Into a 50 mL single-neck round-bottom flask containing a well-stirred solution of 3,6-difluoro-2-[3-(8-hydroxy-1-oxaspiro[4.5]decan-3-yl)-4-oxo-quinazolin-6-yl]oxy-benzonitrile (10, 380 mg, 838.04 mol) in anhydrous dichloromethane (10 mL) was added pyridinium chlorochromate (460 mg, 2.13 mmol) at ambient temperature under nitrogen atmosphere. The resulting mixture was stirred at ambient temperature for 16 hours. After completion, the reaction mixture was filtered through celite, and filtrate was concentrated under reduced pressure to get crude residue. The crude residue was purified by flash silica-gel column chromatography eluting with 90-95% ethyl acetate in dichloromethane to get 320 mg of racemic compound (95% pure). This compound was purified by chiral SFC purification column: Chiralpak ASH [(250 mm*30 mm* 5 m); Mobile Phase: CO₂: isopropyl alcohol (70:30), total flow: 70 g/min; wave length: 220 nm; cycle time: 8.2 min; back pressure: 100 bar) to afford 3,6-difluoro-2-[4-oxo-3-[(3S)-8-oxo-1-oxaspiro[4.5]decan-3-yl]quinazolin-6-yl]oxy-benzonitrile (11-S, first eluting peak, 130 mg, 259.18 mol, 31% yield) as a brownish gummy liquid and 3,6-difluoro-2-[4-oxo-3-[(3R)-8-oxo-1-oxaspiro[4.5]decan-3-yl]quinazolin-6-yl]oxy-benzonitrile (11-R, second eluting peak, 110 mg, 233.93 mol, 28% yield) as an off-white solid. LC-MS(ES+): m/z 452.2 [M+H]⁺. SOR analysis: F1 [α]_{d}: 64.4 (CHCl₃, 0.5M); F2 [α]_{d}: −73.04(CHCl₃, 0.5M).

### Step 11

Into a 10 mL sealed tube containing a well-stirred solution of 3,6-difluoro-2-[4-oxo-3-[(3R)-8-oxo-1-oxaspiro[4.5]decan-3-yl]quinazolin-6-yl]oxy-benzonitrile (11-R, 110 mg, 243.67 mol) in anhydrous N,N-dimethylformamide (3.0 mL) were added [methyl(sulfamoyl)amino]ethane (12, 60 mg, 434.19 mol) and Cs₂CO₃ (250 mg, 767.30 mol) at ambient temperature under nitrogen atmosphere. The resulting mixture was heated to 65° C. for 16 hours. After completion of the reaction, water (30 ml) was added to the reaction mixture and extracted with ethyl acetate (3×50 mL). The combined organic layers were dried over Na₂SO₄, filtered, and concentrated under reduced pressure to get crude residue. The crude residue was purified by flash silica-gel column chromatography eluted with 5-10% of methanol in dichloromethane to afford 6-[2-cyano-3-[[ethyl(methyl)sulfamoyl]amino]-6-fluoro-phenoxy]-4-oxo-3-[(3R)-8-oxo-1-oxaspiro[4.5]decan-3-yl]quinazoline (A-16, 85 mg, 110.43 mol, 45% yield) as a brownish gummy liquid. LC-MS(ES+): m/z 570.2 [M+H]⁺.

### Synthesis A17: Synthesis of 6-[2-cyano-6-fluoro-3-[[2-hydroxyethyl(methyl)sulfamoyl]amino]phenoxy]-4-oxo-3-[(3R)-8-oxo-1-oxaspiro[4.5]decan-3-yl]quinazoline (A-17)

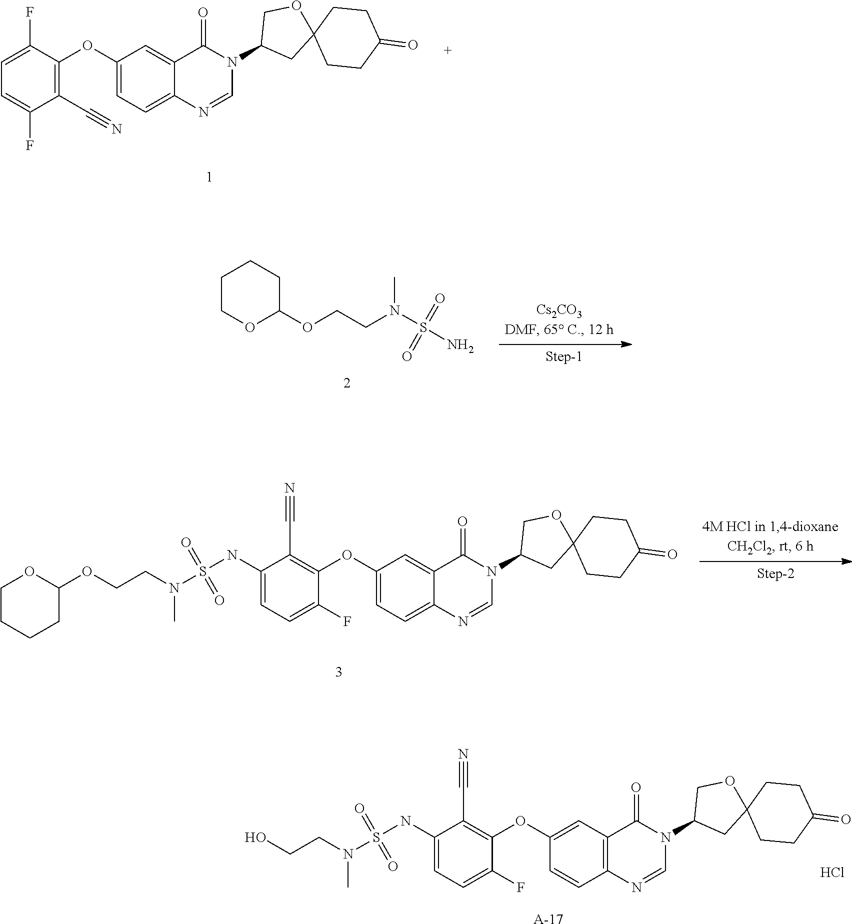

### Step 1

Into a 20 mL sealed-vial containing a well-stirred solution of 3,6-difluoro-2-[4-oxo-3-[(3R)-8-oxo-1-oxaspiro[4.5]decan-3-yl]quinazolin-6-yl]oxy-benzonitrile (1, 750 mg, 1.66 mmol) in N,N-dimethylformamide (10 mL) were added 2-[2-[methyl(sulfamoyl)amino]ethoxy]tetrahydropyran (2, 475.11 mg, 1.99 mmol) and Cs₂CO₃ (1.08 g, 3.32 mmol) at ambient temperature and stirred for at 65° C. for 12 hours. After completion of the reaction, the mixture was filtered through celite and washed with ethyl acetate (20 mL) and water (25 mL) and extracted with ethyl acetate (2×25 mL). The combined organic layers were dried over Na₂SO₄, filtered and concentrated under reduced pressure to afford 6-[2-cyano-6-fluoro-3-[[methyl(2-tetrahydropyran-2-yloxyethyl)sulfamoyl]amino]phenoxy]-4-oxo-3-[(3R)-8-oxo-1-oxaspiro[4.5]decan-3-yl]quinazoline (3, 900 mg, 983.02 mol, 59% yield) as a brown gummy liquid. LC-MS (ES+): m/z 670.3 [M+H]⁺.

### Step 2

Into a 50 mL single-neck round-bottom flask containing a well-stirred solution of 6-[2-cyano-6-fluoro-3-[[methyl(2-tetrahydropyran-2-yloxyethyl)sulfamoyl]amino]phenoxy]-4-oxo-3-[(3R)-8-oxo-1-oxaspiro[4.5]decan-3-yl]quinazoline (3, 900.00 mg, 1.34 mmol) in anhydrous dichloromethane (10 mL) was added 4M HCl in 1,4-dioxane (4 M, 3.36 mL) at 0° C. The resulting mixture was stirred at room temperature for 6 hours. After completion of the reaction, solvents were removed under reduced pressure to get crude 6-[2-cyano-6-fluoro-3-[[2-hydroxyethyl(methyl)sulfamoyl]amino]phenoxy]-4-oxo-3-[(3R)-8-oxo-1-oxaspiro[4.5]decan-3-yl]quinazoline (A-17, 800 mg, 689.32 mol, 51% yield, HCl salt) as a gummy liquid. LC-MS (ES+): m/z 586.3 [M+H]⁺.

### Synthesis A18: Synthesis of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18)

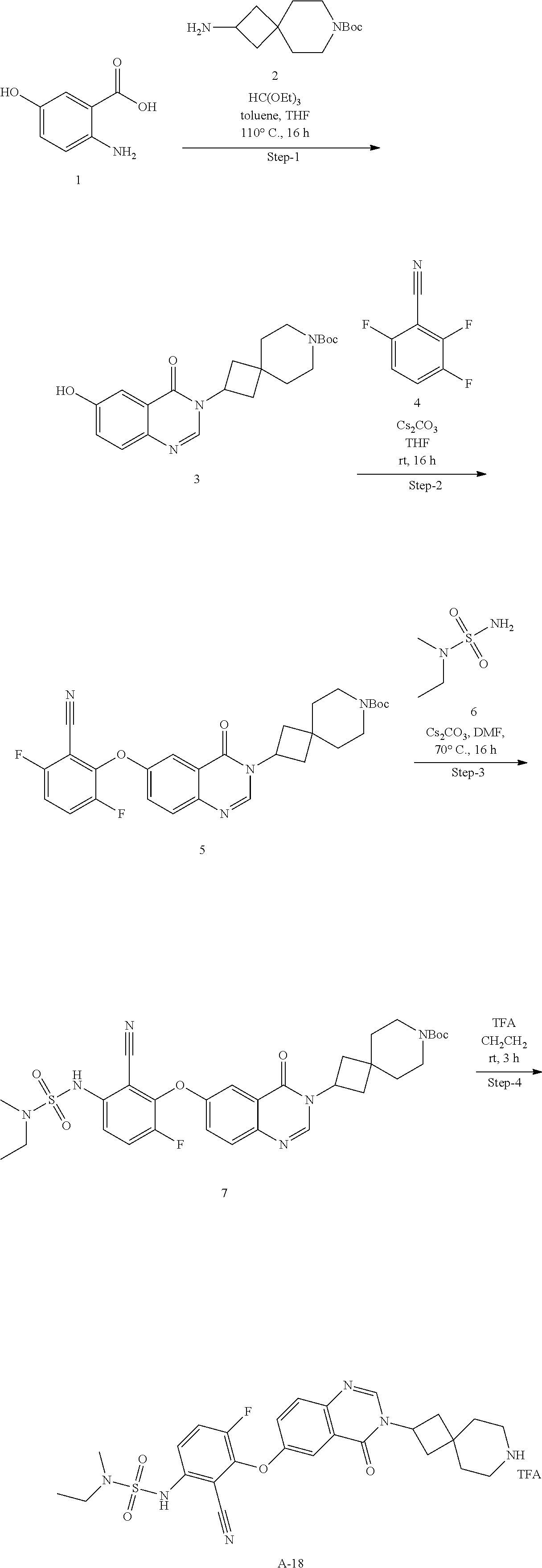

### Step 1

Into a 1 L three-neck round bottom flask containing a solution of 2-amino-5-hydroxy-benzoic acid (1, 16 g, 104.48 mmol) and tert-butyl 2-amino-7-azaspiro[3.5]nonane-7-carboxylate (2, 25.11 g, 104.48 mmol) in anhydrous toluene (200 mL) and anhydrous THF (50.00 mL) was added triethylorthoformate (23.23 g, 156.72 mmol, 26.07 mL) at room temperature under nitrogen atmosphere and reaction mixture was stirred at 110° C. for 16 hours. The reaction mixture was diluted with water (200 mL) and extracted with ethyl acetate (2×1000 mL). Organic layer was washed with NaHCO₃ solution (600 mL) followed by saturated NaCl solution (600 mL), dried over anhydrous Na₂SO₄ and concentrated under reduced pressure to get a crude residue which was triturated with hexane to afford tert-butyl 2-(6-hydroxy-4-oxo-quinazolin-3-yl)-7-azaspiro[3.5]nonane-7-carboxylate (3, 20 g, 51.24 mmol, 49% yield) as a brown solid. UPLC-MS (ES+): m/z 386.2 [M+H]⁺.

### Step 2

Into a 500 mL three-neck round bottom flask containing a solution of tert-butyl 2-(6-hydroxy-4-oxo-quinazolin-3-yl)-7-azaspiro[3.5]nonane-7-carboxylate (3, 16.3 g, 42.29 mmol) in anhydrous THF (65.68 mL) was added Cs₂CO₃ (41.33 g, 126.86 mmol) followed by 2,3,6-trifluorobenzonitrile (4, 50.75 mmol, 5.86 mL). Reaction mixture was stirred at room temperature under nitrogen atmosphere for 16 hours. After completion of reaction, the reaction mixture was diluted with water (600 mL) and extracted with ethyl acetate (2×800 mL). Combined organic extracts were washed saturated NaCl solution (600 mL), dried over anhydrous Na₂SO₄ and concentrated under reduced pressure to get a crude residue which was purified by flash silica-gel (100-200 mesh, 340 g) column chromatography using a gradient of 0-100% ethyl acetate/hexane to afford tert-butyl 2-[6-(2-cyano-3,6-difluoro-phenoxy)-4-oxo-quinazolin-3-yl]-7-azaspiro[3.5]nonane-7-carboxylate (5, 15 g, 26.44 mmol, 63% yield) as an off-white solid. UPLC-MS (ES+): m/z 523.2 [M+H]⁺.

### Step 3

Into a 500 mL single-neck round bottom flask containing a well stirred solution of tert-butyl 2-[6-(2-cyano-3,6-difluoro-phenoxy)-4-oxo-quinazolin-3-yl]-7-azaspiro[3.5]nonane-7-carboxylate (5, 5 g, 8.80 mmol) in anhydrous N,N-dimethylformamide (80 mL) was added Cs₂CO₃ (11.47 g, 35.21 mmol) and [methyl(sulfamoyl)amino]ethane (6, 3.65 g, 26.41 mmol) at room temperature under nitrogen atmosphere. The reaction mixture was stirred at 70° C. under nitrogen atmosphere for 16 hours. After completion of the reaction, the reaction mixture was allowed to cool to room temperature and water (100 mL) was added. Precipitated solid was filtered and filtrate was extracted with ethyl acetate (3×300 mL). Combined organic layer was washed with cold water (100 mL), dried over anhydrous Na₂SO₄, filtered and concentrated under reduced pressure to afford crude tert-butyl 2-[6-[2-cyano-3-[[ethyl(methyl)sulfamoyl]amino]-6-fluoro-phenoxy]-4-oxo-quinazolin-3-yl]-7-azaspiro[3.5]nonane-7-carboxylate (7, 5.5 g, 6.28 mmol, 71% yield) as a brown gummy liquid. UPLC-MS (ES+): m/z 641.3 [M+H]⁺.

### Step 4

Into a 500 mL single-neck round bottom flask containing a well stirred solution of tert-butyl 2-[6-[2-cyano-3-[[ethyl(methyl)sulfamoyl]amino]-6-fluoro-phenoxy]-4-oxo-quinazolin-3-yl]-7-azaspiro[3.5]nonane-7-carboxylate (7, 11.7 g, 13.37 mmol) in anhydrous dichloromethane (200 mL) was added trifluoroacetic acid (200.50 mmol, 15.34 mL) at 0° C. under nitrogen atmosphere. The resulting mixture was stirred at room temperature for 3 hours. After completion of reaction, the reaction mixture was concentrated under reduced pressure to get a crude residue which was triturated with methyl tert-butyl ether (2×50 mL) and dried under reduced pressure to afford 3-(7-azaspiro[3.5]nonan-2-yl)-6-[2-cyano-3-[[ethyl(methyl)sulfamoyl]amino]-6-fluoro-phenoxy]-4-oxo-quinazoline (A-18, 10.3 g, 13.21 mmol, 99% yield, TFA salt) as a brown gummy liquid. UPLC-MS (ES+): m/z 541.2 [M+H]⁺.

### Synthesis A19: Synthesis of N-[3-[3-(7-azaspiro[3.5]nonan-2-yl)-4-oxo-quinazolin-6-yl]oxy-2-cyano-4-fluoro-phenyl]propane-2-sulfonamide (A-19)

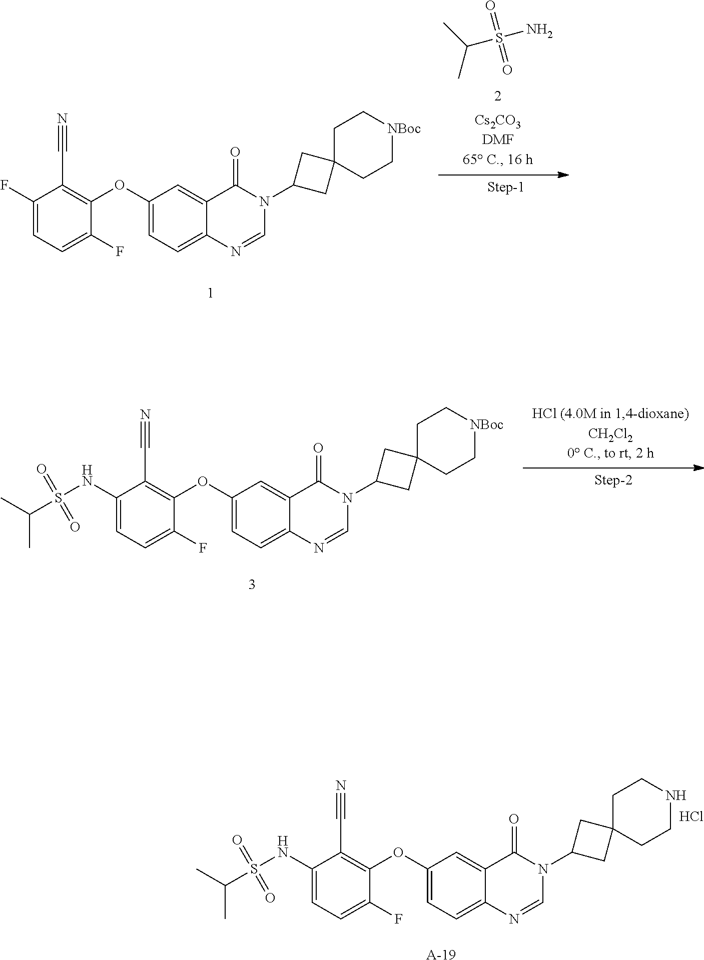

### Step 1

Into a 20 mL pressure vial containing a well-stirred solution of tert-butyl 2-[6-(2-cyano-3,6-difluoro-phenoxy)-4-oxo-quinazolin-3-yl]-7-azaspiro[3.5]nonane-7-carboxylate (1, 600.00 mg, 1.15 mmol) in N,N-dimethylformamide (10 mL) were added propane-2-sulfonamide (2, 282.86 mg, 2.30 mmol) and Cs₂CO₃ (935.29 mg, 2.87 mmol) at room temperature under nitrogen atmosphere. The reaction mixture was stirred at 65° C. for 16 hours. After completion of the reaction, the reaction mixture was cooled to room temperature and diluted with water (50 mL) and extracted with ethyl acetate (3×100 mL). The combined organic layers were washed with cold water (3×30 mL), dried over anhydrous Na₂SO₄, filtered and concentrated under reduced pressure to afford crude residue, which was purified by flash silica-gel (100-200 mesh) column with 0-100% ethyl acetate/hexane as an eluent to afford tert-butyl 2-[6-[2-cyano-6-fluoro-3-(isopropylsulfonylamino)phenoxy]-4-oxo-quinazolin-3-yl]-7-azaspiro[3.5]nonane-7-carboxylate (3, 700 mg, 1.11 mmol, 96% yield) as a light brown solid. UPLC-MS (ES+): m/z 626.2 [M+H]⁺.

### Step 2

Into a 50 mL single neck round-bottom flask containing a well-stirred solution of tert-butyl 2-[6-[2-cyano-6-fluoro-3-(isopropylsulfonylamino)phenoxy]-4-oxo-quinazolin-3-yl]-7-azaspiro[3.5]nonane-7-carboxylate (3, 700 mg, 1.10 mmol) in dichloromethane (10 mL) was added 4.0 M HCl in 1,4-dioxane (4 M, 2.74 mL) slowly at 0° C. under nitrogen atmosphere. The resulting solution was stirred at room temperature for 2 hours. After completion of the reaction, the reaction mixture was concentrated directly under reduced pressure to afford N-[3-[3-(7-azaspiro[3.5]nonan-2-yl)-4-oxo-quinazolin-6-yl]oxy-2-cyano-4-fluoro-phenyl]propane-2-sulfonamide (A-19, 550 mg, 921.31 mol, 84% yield, HCl salt) as an off white solid. UPLC-MS (ESI): m/z 526.2 [M+H]⁺.

### Synthesis A20: Synthesis of N-[3-[3-(7-azaspiro[3.5]nonan-2-yl)-4-oxo-quinazolin-6-yl]oxy-2-cyano-4-fluoro-phenyl]cyclopropanesulfonamide (A-20)

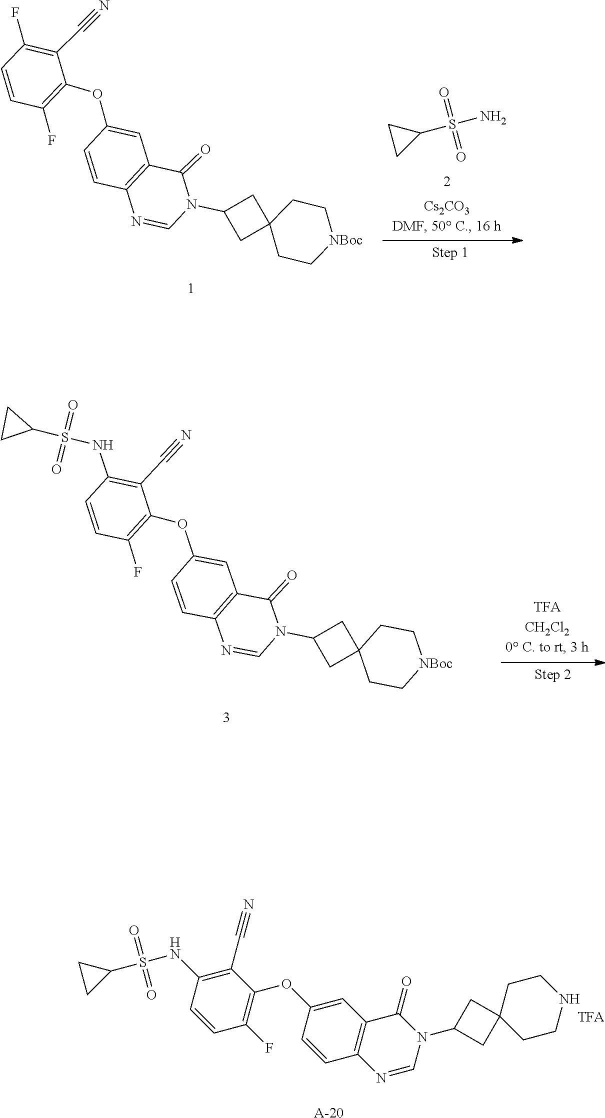 [SMILES: CC(C)(C)OC(=O)N1CCC2(CC1)CC(n1cnc3ccc(Oc4c(F)ccc(F)c4C#N)cc3c1=O)C2 ; NS(=O)(=O)C1CC1 ; CC(C)(C)OC(=O)N1CCC2(CC1)CC(n1cnc3ccc(Oc4c(F)ccc(NS(=O)(=O)C5CC5)c4C#N)cc3c1=O)C2 ; N#Cc1c(NS(=O)(=O)C2CC2)ccc(F)c1Oc1ccc2ncn(C3CC4(CCNCC4)C3)c(=O)c2c1 ; "TFA" ; "A-20"]

### Step 1

Into a 20 mL glass vial with pressure released cap containing a well-stirred solution of tert-butyl 2-[6-(2-cyano-3,6-difluoro-phenoxy)-4-oxo-quinazolin-3-yl]-7-azaspiro[3.5]nonane-7-carboxylate (1, 300 mg, 562.63 μmol) and cyclopropanesulfonamide (2, 102.25 mg, 843.95 mol) in N,N-dimethylformamide (5 mL) was added Cs₂CO₃ (458.29 mg, 1.41 mmol) at room temperature. Reaction mixture was stirred at 50° C. for 16 hours. After completion of the reaction, the reaction mixture was diluted with ice cold water (30 mL) and stirred for 10 min, the resulting solid precipitate (fluorescent impurity) was filtered through Buchner funnel, filtrate was extracted with ethyl acetate (2×50 mL). Combined organic layer was dried over anhydrous Na₂SO₄, filtered and concentrated under reduced pressure to afford tert-butyl 2-[6-[2-cyano-3-(cyclopropylsulfonylamino)-6-fluoro-phenoxy]-4-oxo-quinazolin-3-yl]-7-azaspiro[3.5]nonane-7-carboxylate (350 mg, 534.01 mol, 95% yield) as a light brown gummy liquid. UPLC-MS (ES+): m/z 624.2 [M+H]⁺.

### Step 2

Into a 100 mL single-neck round-bottom flask containing a well stirred solution of tert-butyl 2-[6-[2-cyano-3-(cyclopropylsulfonylamino)-6-fluoro-phenoxy]-4-oxo-quinazolin-3-yl]-7-azaspiro[3.5]nonane-7-carboxylate (3, 350 mg, 533.11 mol) in dichloromethane (10 mL) was added trifluoroacetic acid (5.33 mmol, 407.96 L) at 0° C. Reaction mixture was stirred at room temperature for 3 hours. After consumption of the starting material, the reaction mixture was concentrated under reduced pressure to get crude residue, which was triturated with methyl tert-butyl ether (2×20 mL) and dried to afford N-[3-[3-(7-azaspiro[3.5]nonan-2-yl)-4-oxo-quinazolin-6-yl]oxy-2-cyano-4-fluoro-phenyl]cyclopropanesulfonamide (A-20, 300 mg, 393.91 mol, 74% yield, TFA salt) as a brown semi solid. UPLC-MS (ES+): m/z 524.4 [M+H]⁺.

### Synthesis A21: Synthesis of N-[3-[3-(7-azaspiro[3.5]nonan-2-yl)-4-oxo-quinazolin-6-yl]oxy-2-cyano-4-fluoro-phenyl]cyclobutanesulfonamide (A-21)

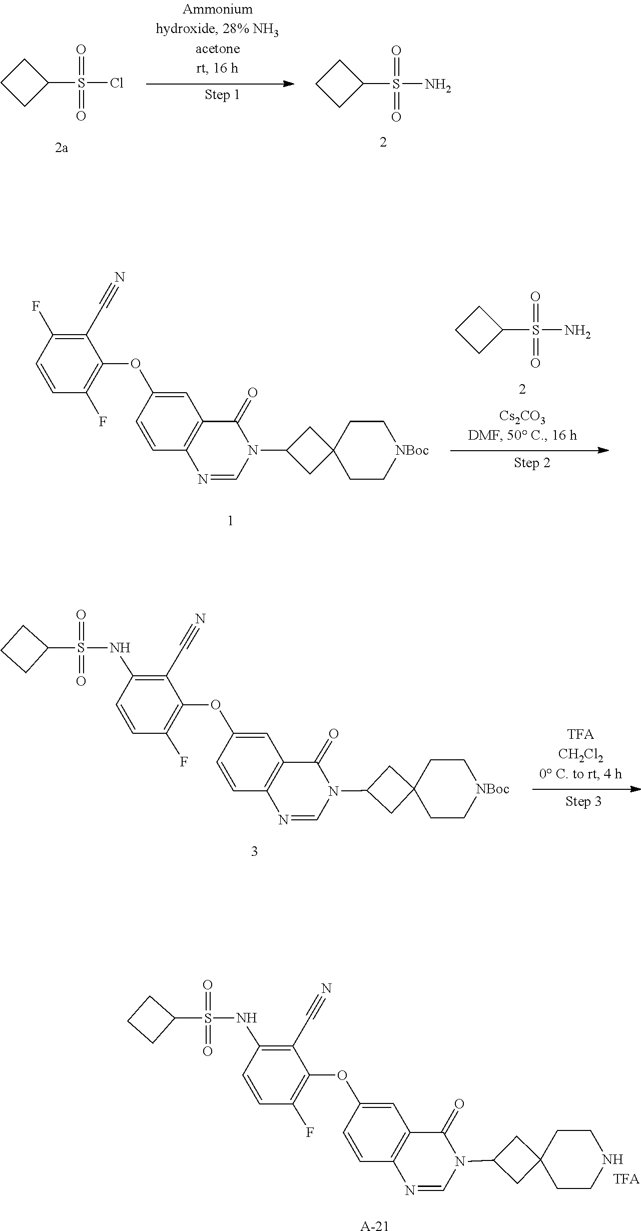

### Step 1

Into a 25 mL two-neck round-bottom flask containing a well stirred solution of cyclobutanesulfonyl chloride (2a, 0.5 g, 3.23 mmol) in acetone (10 mL) was added aqueous ammonium hydroxide (32.34 mmol, 1.26 mL) at 0° C. and reaction mixture was stirred at room temperature for 16 hours. After completion of the reaction, the reaction mixture was quenched with water (20 mL) at 0° C. and extracted with ethyl acetate (3×20 mL). Combined organic layer was washed with 10% NaHCO₃ solution (20 mL), dried over anhydrous Na₂SO₄, filtered and concentrated under reduced pressure to get a crude residue which was triturated with dichloromethane (5 mL) and hexane (5 mL), dried under reduced pressure to afford cyclobutanesulfonamide (2, 0.250 g, 1.85 mmol, 57% yield) as a brown gummy liquid. ¹H NMR (400 MHz, DMSO-d₆): δ 6.72 (s, 1H), 3.74-3.70 (m, 1H), 2.28-2.10 (m, 4H), 1.91-1.79 (m, 2H).

### Step 2

Into a 40 mL pressure vial containing a well stirred solution of tert-butyl 2-[6-(2-cyano-3,6-difluoro-phenoxy)-4-oxo-quinazolin-3-yl]-7-azaspiro[3.5]nonane-7-carboxylate (1, 0.400 g, 765.49 μmol) and cyclobutanesulfonamide (2, 258.71 mg, 1.91 mmol) in N,N-dimethylformamide (10 mL) was added Cs₂CO₃ (249.41 mg, 765.49 mol) at room temperature under nitrogen atmosphere. Resulting reaction mixture was stirred at 50° C. for 16 hours. After completion of the reaction, the reaction mixture was diluted with water (50 mL) and extracted with ethyl acetate (3×30 mL). Combined organic layer was washed with water (30 mL), saturated NaCl solution (30 mL), dried over anhydrous Na₂SO₄, filtered and concentrated under reduced pressure to afford a crude residue which was purified by flash (25 g, 230-400 silica-gel mesh) column chromatography using 0-100% ethyl acetate in hexane as an eluent to afford tert-butyl 2-[6-[2-cyano-3-(cyclobutylsulfonylamino)-6-fluoro-phenoxy]-4-oxo-quinazolin-3-yl]-7-azaspiro[3.5]nonane-7-carboxylate (3, 0.250 g, 337.14 mol, 44% yield) as an off-white solid. UPLC-MS (ES−): m/z 636.0 [M−H]⁻.

### Step 3

Into a 25 mL single-neck round-bottom flask containing a well stirred solution of tert-butyl 2-[6-[2-cyano-3-(cyclobutylsulfonylamino)-6-fluoro-phenoxy]-4-oxo-quinazolin-3-yl]-7-azaspiro[3.5]nonane-7-carboxylate (3, 0.250 g, 392.02 mol) in dichloromethane (10 mL) was added trifluoroacetic acid (5.94 mmol, 454.55 L) at 0° C. under nitrogen atmosphere. Resulting reaction mixture was stirred at room temperature for 4 hours. After completion of the reaction, the reaction mixture was concentrated under reduced pressure to afford a crude N-[3-[3-(7-azaspiro[3.5]nonan-2-yl)-4-oxo-quinazolin-6-yl]oxy-2-cyano-4-fluoro-phenyl]cyclobutanesulfonamide (A-21, 0.210 g, 315.82 mol, 81% yield, TFA salt) as an off-white solid. UPLC-MS (ES+): m/z 538.3 [M+H]⁺.

### Synthesis A22: Synthesis of N-[3-[3-(7-azaspiro[3.5]nonan-2-yl)-4-oxo-quinazolin-6-yl]oxy-2-cyano-4-fluoro-phenyl]cyclopentanesulfonamide (A-22)

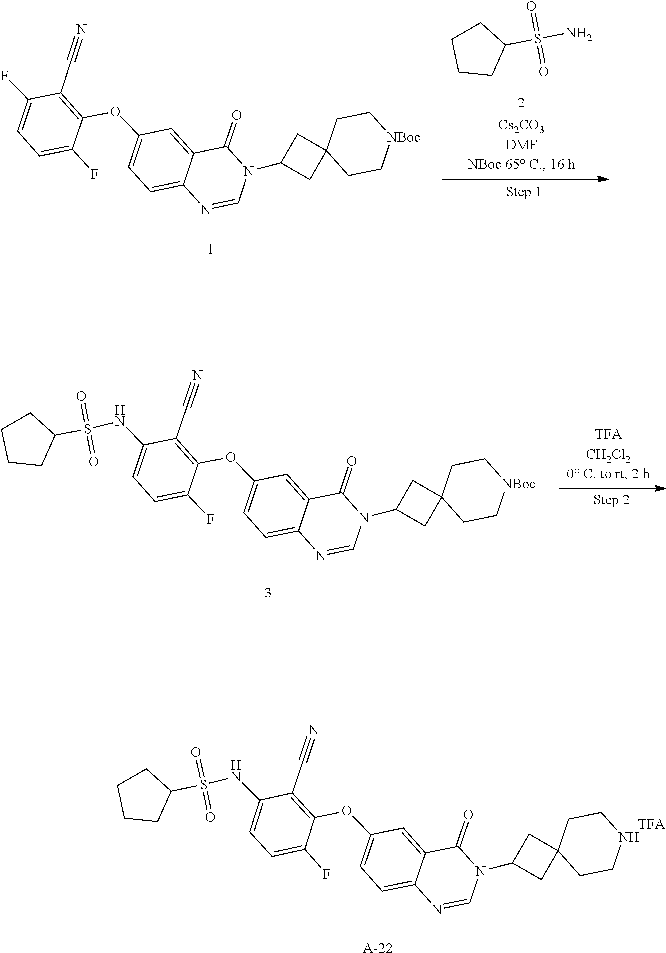 [SMILES: CC(C)(C)OC(=O)N1CCC2(CC1)CC(n1cnc3ccc(Oc4c(F)ccc(F)c4C#N)cc3c1=O)C2 ; NS(=O)(=O)C1CCCC1 ; CC(C)(C)OC(=O)N1CCC2(CC1)CC(n1cnc3ccc(Oc4c(F)ccc(NS(=O)(=O)C5CCCC5)c4C#N)cc3c1=O)C2 ; N#Cc1c(NS(=O)(=O)C2CCCC2)ccc(F)c1Oc1ccc2ncn(C3CC4(CCNCC4)C3)c(=O)c2c1 ; "TFA" ; "A-22"]

### Step 1

Into a 100 mL single-neck round-bottom flask containing a well-stirred solution of tert-butyl 2-[6-(2-cyano-3,6-difluoro-phenoxy)-4-oxo-quinazolin-3-yl]-7-azaspiro[3.5]nonane-7-carboxylate (1, 500 mg, 956.86 μmol) in anhydrous N,N-dimethylformamide (15 mL) was added Cs₂CO₃ (935.29 mg, 2.87 mmol) followed by cyclopentanesulfonamide (2,285.55 mg, 1.91 mmol) at room temperature under nitrogen atmosphere. Reaction mixture was stirred at 65° C. under nitrogen atmosphere for 16 hours. After completion of the reaction, the reaction mixture was diluted with water and precipitated solid filtered through sintered funnel and washed with water. The obtained filtrate was extracted with ethyl acetate (2×70 mL) and washed with saturated NaCl solution (50 mL). Combined organic layers were dried over anhydrous Na₂SO₄, filtered and concentrated under reduced pressure to afford tert-butyl 2-[6-[2-cyano-3-(cyclopentylsulfonylamino)-6-fluoro-phenoxy]-4-oxo-quinazolin-3-yl]-7-azaspiro[3.5]nonane-7-carboxylate (3, 430 mg, 463.68 μmol, 48% yield) as brown gummy liquid. UPLC-MS (ES+): m/z 652.2 [M+H]⁺.

### Step 2

Into a 100 mL single-neck round-bottom flask containing a well-stirred solution of tert-butyl 2-[6-[2-cyano-3-(cyclopentylsulfonylamino)-6-fluoro-phenoxy]-4-oxo-quinazolin-3-yl]-7-azaspiro[3.5]nonane-7-carboxylate (3, 430.00 mg, 461.84 μmol) in anhydrous dichloromethane (6 mL) was added trifluoroacetic acid (9.24 mmol, 706.85 μL) at 0° C. The resulting mixture was stirred at room temperature for 2 hours. After completion of reaction, solvents were removed from the reaction mixture under reduced pressure to get a crude, which was triturated with methyl tert-butyl ether (20 mL) and dried under reduced pressure to get N-[3-[3-(7-azaspiro[3.5]nonan-2-yl)-4-oxo-quinazolin-6-yl]oxy-2-cyano-4-fluoro-phenyl]cyclopentanesulfonamide (A-22, 350 mg, 425.58 μmol, 92% yield, TFA salt) as a brown solid. UPLC-MS (ES+): m/z 552.2 [M+H]⁺.

### Synthesis A23: Synthesis of (3R)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-23)

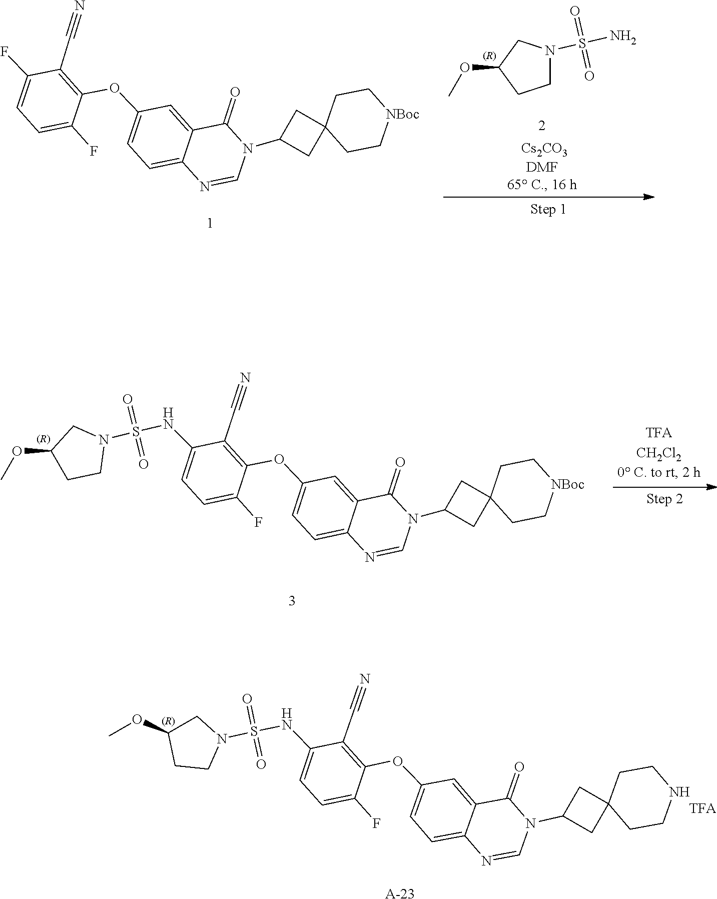

### Step 1

Into a 20 mL pressure vial containing a well-stirred solution of tert-butyl 2-[6-(2-cyano-3,6-difluoro-phenoxy)-4-oxo-quinazolin-3-yl]-7-azaspiro[3.5]nonane-7-carboxylate (1, 1.2 g, 2.25 mmol) in N,N-dimethylformamide (10 mL) were added (3R)-3-methoxypyrrolidine-1-sulfonamide (2, 608.40 mg, 3.38 mmol) and Cs₂CO₃ (1.83 g, 5.63 mmol) at room temperature under nitrogen atmosphere. The reaction mixture was stirred at 65° C. for 16 hours. After completion of the reaction, the reaction mixture was cooled to room temperature and diluted with water (50 mL) and extracted with ethyl acetate (3×100 mL). The combined organic layers were washed with cold water (3×30 mL), dried over anhydrous Na₂SO₄, filtered and concentrated under reduced pressure to afford crude residue, which was purified by flash silica-gel (100-200 mesh) column with 0-100% ethyl acetate/hexane as an eluent to afford tert-butyl 2-[6-[2-cyano-6-fluoro-3-[[(3R)-3-methoxypyrrolidin-1-yl]sulfonylamino]phenoxy]-4-oxo-quinazolin-3-yl]-7-azaspiro[3.5]nonane-7-carboxylate (3, 750 mg, 959.63 mol, 43% yield) as an off-white solid. UPLC-MS (ES+): m/z 683.2 [M+H]⁺.

### Step 2

Into a 50 mL single neck round-bottom flask containing a well-stirred solution of tert-butyl 2-[6-[2-cyano-6-fluoro-3-[[(3R)-3-methoxypyrrolidin-1-yl]sulfonylamino]phenoxy]-4-oxo-quinazolin-3-yl]-7-azaspiro[3.5]nonane-7-carboxylate (3, 750 mg, 1.10 mmol) in dichloromethane (10 mL) was added trifluoroacetic acid (21.97 mmol, 1.68 mL) slowly at 0° C. under nitrogen atmosphere. The resulting solution was stirred at room temperature for 2 hours. After completion of the reaction, the reaction mixture was concentrated directly under reduced pressure to get crude residue which was triturated with methyl tert-butyl ether (2×30 mL) and dried to afford (3R)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-23, 760 mg, 1.02 mmol, 93% yield, TFA salt) as a brown semi solid. UPLC-MS (ES+): m/z 583.1 [M+H]⁺.

### Synthesis A24: Synthesis of (3S)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-24)

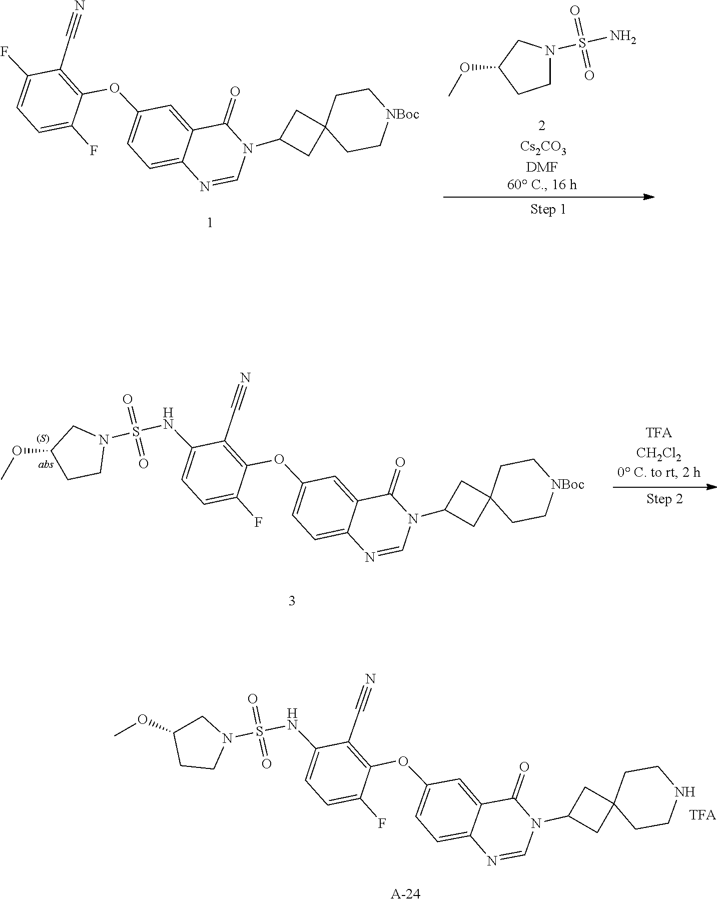

### Step 1

Into a 40 mL pressure vial containing a well-stirred solution of tert-butyl 2-[6-(2-cyano-3,6-difluoro-phenoxy)-4-oxo-quinazolin-3-yl]-7-azaspiro[3.5]nonane-7-carboxylate (1, 800 mg, 1.53 mmol) in N,N-dimethylformamide (10 mL) were added (3S)-3-methoxypyrrolidine-1-sulfonamide (2, 413.88 mg, 2.30 mmol) and Cs₂CO₃ (1.25 g, 3.83 mmol) at room temperature under nitrogen atmosphere and stirred at 60° C. for 16 hours. After completion of reaction, the reaction mixture was diluted with water (50 mL) and extracted with ethyl acetate (2×50 mL). The combined organic layers were washed with saturated NaCl solution (50 mL) and dried over anhydrous Na₂SO₄, filtered and concentrated under reduced pressure to get a crude residue, which was purified by flash silica-gel (100-200 mesh) column chromatography using the gradient of 0-100% ethyl acetate/hexane to afford tert-butyl 2-[6-[2-cyano-6-fluoro-3-[[(3S)-3-methoxypyrrolidin-1-yl]sulfonylamino]phenoxy]-4-oxo-quinazolin-3-yl]-7-azaspiro[3.5]nonane-7-carboxylate (3, 700 mg, 1.03 mmol, 67% yield) as an off-white solid. UPLC-MS (ES+): m/z 683.2 [M+H]⁺.

### Step 2

Into a 50 mL single-neck round-bottom flask containing a well-stirred solution of tert-butyl 2-[6-[2-cyano-6-fluoro-3-[[(3S)-3-methoxypyrrolidin-1-yl]sulfonylamino]phenoxy]-4-oxo-quinazolin-3-yl]-7-azaspiro[3.5]nonane-7-carboxylate (3, 700.00 mg, 1.03 mmol) in anhydrous dichloromethane (10 mL) was added trifluoroacetic acid (10.25 mmol, 784.56 L) slowly at 0° C. under nitrogen atmosphere. The reaction mixture was stirred at room temperature for 2 hours. After completion of reaction, the reaction mixture was concentrated under reduced pressure get a crude residue, which was triturated with methyl tert-butyl ether (2×10 mL) to remove the non-polar impurity to afford (3S)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-24, 600 mg, 740.67 mol, 72% yield, TFA salt) as an off-white solid. UPLC-MS (ES+): m/z 583.2 [M+H]⁺.

### Synthesis A25: Synthesis of (3R)-N-[3-[3-(7-azaspiro[3.5]nonan-2-yl)-4-oxo-quinazolin-6-yl]oxy-2-cyano-4-fluoro-phenyl]-3-fluoro-pyrrolidine-1-sulfonamide (A-25)

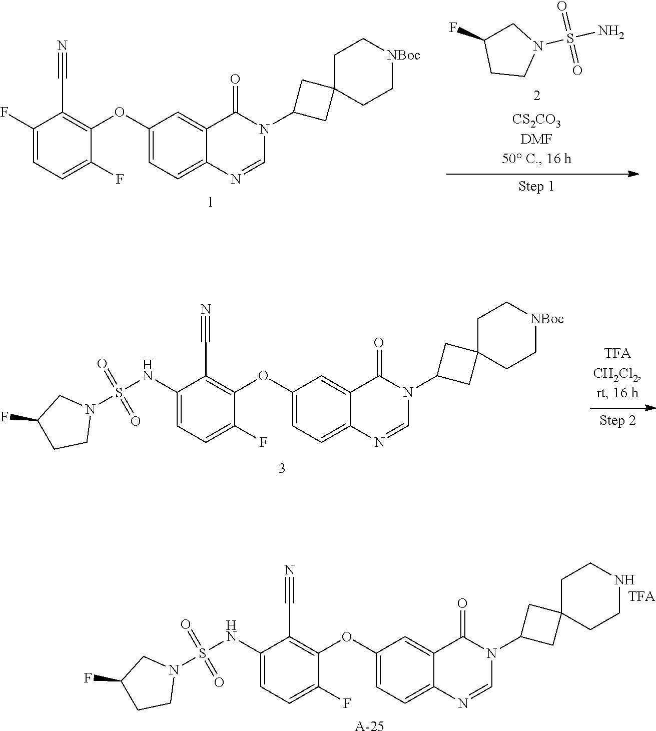

### Step 1

Into a 50 mL two-neck round-bottom flask containing a well-stirred solution of tert-butyl 2-[6-(2-cyano-3,6-difluoro-phenoxy)-4-oxo-quinazolin-3-yl]-7-azaspiro[3.5]nonane-7-carboxylate (1, 600 mg, 1.15 mmol) in N,N-dimethylformamide (6 mL) were added Cs₂CO₃ (1.12 g, 3.44 mmol) and (3R)-3-fluoropyrrolidine-1-sulfonamide (2, 231.74 mg, 1.38 mmol) at room temperature. Resulting reaction mixture was stirred at 50° C. for 16 hours. After completion of the reaction, the reaction mixture was quenched with water (30 mL), filtered through sintered funnel and filtrate was extracted with ethyl acetate (2×30 mL). Combined organic layer was dried over anhydrous Na₂SO₄, filtered and concentrated to afford a crude tert-butyl 2-[6-[2-cyano-6-fluoro-3-[[(3R)-3-fluoropyrrolidin-1-yl]sulfonylamino]phenoxy]-4-oxo-quinazolin-3-yl]-7-azaspiro[3.5]nonane-7-carboxylate (3, 600 mg, 661.97 mol, 58% yield) as a brown gummy. UPLC-MS (ES+): m/z 671.3 [M+H]⁺.

### Step 2

Into a 50 mL single-neck round-bottom flask containing a well-stirred solution of tert-butyl 2-[6-[2-cyano-6-fluoro-3-[[(3R)-3-fluoropyrrolidin-1-yl]sulfonylamino]phenoxy]-4-oxo-quinazolin-3-yl]-7-azaspiro[3.5]nonane-7-carboxylate (3, 600 mg, 894.55 mol) in dichloromethane (10 mL) was added trifluoroacetic acid (4.47 mmol, 342.27 L) at 0° C. under nitrogen atmosphere. Resulting reaction mixture was stirred at room temperature for 16 hours. After completion of the reaction, the reaction mixture was concentrated under reduced pressure and co-distilled with methyl tert-butyl ether (20 mL) to afford a crude (3R)-N-[3-[3-(7-azaspiro[3.5]nonan-2-yl)-4-oxo-quinazolin-6-yl]oxy-2-cyano-4-fluoro-phenyl]-3-fluoro-pyrrolidine-1-sulfonamide (A-25, 500 mg, 675.54 mol, 76% yield, TFA salt) as a brown gummy. UPLC-MS (ES+): m/z 571.3 [M+H]⁺.

### Synthesis A26: Synthesis of N-[3-[3-(7-azaspiro[3.5]nonan-2-yl)-4-oxo-quinazolin-6-yl]oxy-2-cyano-4-fluoro-phenyl]morpholine-4-sulfonamide (A-26)

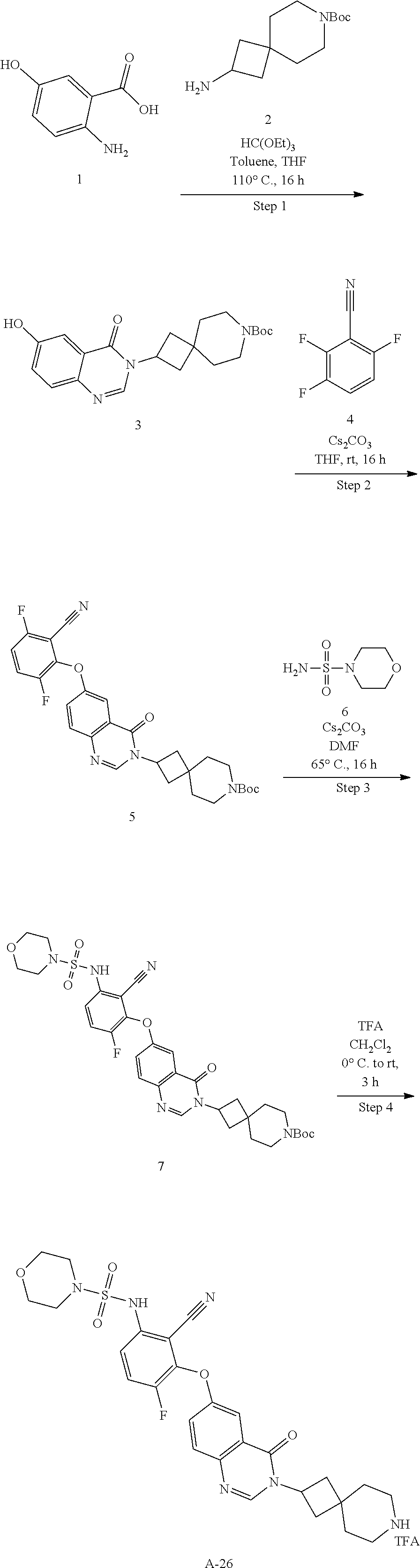

### Step 1

Into a 1000 mL three-neck round-bottom flask containing a well stirred solution of 2-amino-5-hydroxy-benzoic acid (1, 15 g, 97.95 mmol), tert-butyl 2-amino-7-azaspiro[3.5]nonane-7-carboxylate (2, 23.54 g, 97.95 mmol) in Toluene (300 mL) and THF (60 mL) was added triethyl orthoformate (146.93 mmol, 24.44 mL) and resulting mixture stirred at 110° C. for 16 hours. After completion of the reaction, the reaction mixture was diluted with water (250 mL) and extracted with ethyl acetate (3×250 mL). The organic layer was washed with NaHCO₃ solution (300 mL), saturated NaCl (250 mL), dried over anhydrous Na₂SO₄ and concentrated under reduced pressure to get crude residue, which was triturated with hexane to afford tert-butyl 2-(6-hydroxy-4-oxo-quinazolin-3-yl)-7-azaspiro[3.5]nonane-7-carboxylate (3, 18.5 g, 41.76 mmol, 43% yield) as a brown solid. UPLC-MS (ES+): m/z 386.10 [M+H]⁺.

### Step 2

Into a 500 mL three-neck round-bottom flask containing a well stirred solution of tert-butyl 2-(6-hydroxy-4-oxo-quinazolin-3-yl)-7-azaspiro[3. 5]nonane-7-carboxylate (3, 18.5 g, 41.76 mmol) in THF (250 mL) were added Cs₂CO₃ (68.02 g, 208.78 mmol) and 2,3,6-trifluorobenzonitrile (4, 50.11 mmol, 5.79 mL) at room temperature under nitrogen atmosphere. The reaction mixture was stirred at same temperature for 16 hours. After completion of the reaction, the reaction mixture was diluted with water (100 mL), extracted with ethyl acetate (2×250 mL). The organic layer was washed with saturated NaCl solution (150 mL), the combined organic layers were dried over anhydrous Na₂SO₄ and concentrated under reduced pressure to get crude residue, which was purified by silica gel column chromatography (100-200 mesh) using a gradient of 0-100% ethyl acetate/hexane to afford tert-butyl 2-[6-(2-cyano-3,6-difluoro-phenoxy)-4-oxo-quinazolin-3-yl]-7-azaspiro[3. 5]nonane-7-carboxylate (5, 14.4 g, 27.15 mmol, 65% yield) as an off-white solid. UPLC-MS (ES+): m/z 523.3 [M+H]⁺.

### Step 3

Into a 40 mL pressure vial containing a well stirred solution of tert-butyl 2-[6-(2-cyano-3,6-difluoro-phenoxy)-4-oxo-quinazolin-3-yl]-7-azaspiro[3.5]nonane-7-carboxylate (5, 400 mg, 754.08 mol) and morpholine-4-sulfonamide (6, 395.77 mg, 2.26 mmol) in N,N-dimethylformamide (8 mL) was added Cs₂CO₃ (737.09 mg, 2.26 mmol) at room temperature under nitrogen atmosphere. Reaction mixture was stirred at 65° C. for 16 hours. After completion of the reaction, the reaction mixture was quenched with ice cold water (30 mL) and filtered through Celite pad, filtrate was extracted with ethyl acetate (2×30 mL). Combined organic extracts were dried over anhydrous Na₂SO₄, concentrated under reduced pressure to afford crude residue which was purified by column chromatography by using (100-200 mesh) silica gel to afford tert-butyl 2-[6-[2-cyano-6-fluoro-3 (morpholinosulfonylamino)phenoxy]-4-oxo-quinazolin-3-yl]-7-azaspiro[3.5]nonane-7-carboxylate (7, 270 mg, 333.70 mol, 44% yield) as a brown sticky liquid. UPLC-MS (ES+): m/z 669.20 [M+H]⁺.

### Step 4

Into a 25 mL single-neck round-bottom flask containing a well stirred solution of tert-butyl 2-[6-[2-cyano-6-fluoro-3-(morpholinosulfonylamino)phenoxy]-4-oxo-quinazolin-3-yl]-7-azaspiro[3.5]nonane-7-carboxylate (7, 270 mg, 333.70 mol) in dichloromethane (4 mL) was added trifluoroacetic acid (8.34 mmol, 638.39 L) at 0° C. under nitrogen atmosphere. The reaction mixture was stirred at room temperature for 3 hr. After completion of the reaction, the reaction mixture was concentrated under reduced pressure and triturated with methyl tert-butyl ether to afford N-[3-[3-(7-azaspiro[3.5]nonan-2-yl)-4-oxo-quinazolin-6-yl]oxy-2-cyano-4-fluoro-phenyl]morpholine-4-sulfonamide (A-26, 260 mg, 322.79 mol, 97% yield, TFA salt) as a brown solid. UPLC-MS (ES+): m/z 569.20 [M+H]⁺.

### Synthesis A27: Synthesis of N-[3-[3-(7-azaspiro[3.5]nonan-2-yl)-4-oxo-quinazolin-6-yl]oxy-2-cyano-4-fluoro-phenyl]tetrahydropyran-4-sulfonamide (A-27)

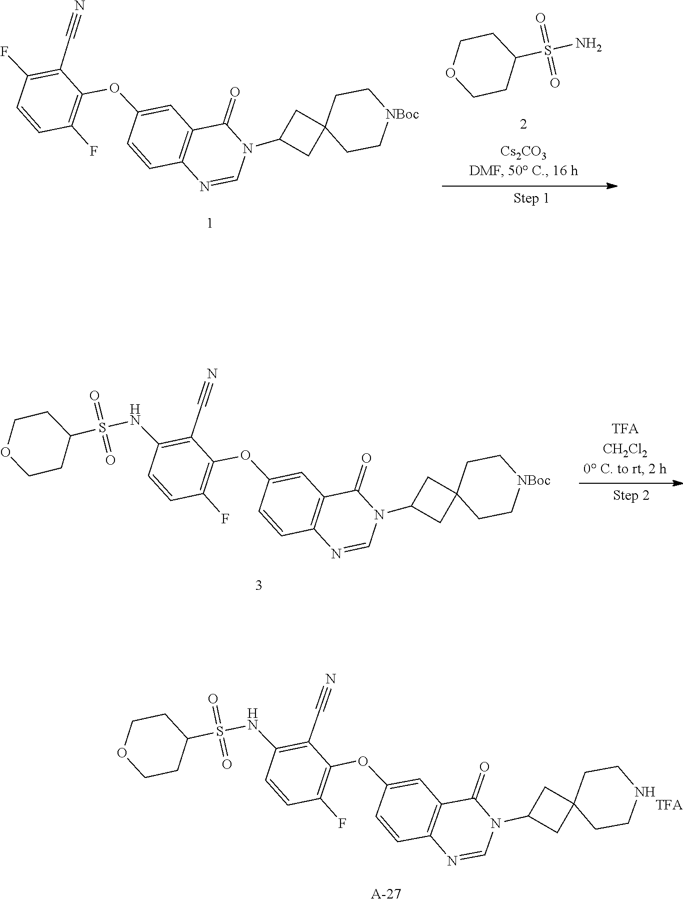 [SMILES: CC(C)(C)OC(=O)N1CCC2(CC1)CC(n1cnc3ccc(Oc4c(F)ccc(F)c4C#N)cc3c1=O)C2 ; NS(=O)(=O)C1CCOCC1 ; CC(C)(C)OC(=O)N1CCC2(CC1)CC(n1cnc3ccc(Oc4c(F)ccc(NS(=O)(=O)C5CCOCC5)c4C#N)cc3c1=O)C2 ; N#Cc1c(NS(=O)(=O)C2CCOCC2)ccc(F)c1Oc1ccc2ncn(C3CC4(CCNCC4)C3)c(=O)c2c1 ; "TFA" ; "A-27"]

### Step 1

Into a 20 mL glass vial containing a well-stirred solution of tert-butyl 2-[6-(2-cyano-3,6-difluoro-phenoxy)-4-oxo-quinazolin-3-yl]-7-azaspiro[3.5]nonane-7-carboxylate (1, 300 mg, 562.63 mol) and tetrahydropyran-4-sulfonamide (2, 158.02 mg, 956.48 mol) in N,N-dimethylformamide (5 mL) was added Cs₂CO₃ (458.29 mg, 1.41 mmol) at room temperature. Reaction mixture was stirred at 50° C. for 16 hours. After completion, the reaction mixture was diluted with water (50 mL) and stirred for 10 minutes, the resulting solid precipitate (fluorescent impurity) was filtered through Buchner funnel and filtrate was extracted with ethyl acetate (2×50 mL). Combined organic layers were dried over anhydrous Na₂SO₄, filtered and concentrated under reduced pressure to afford tert-butyl 2-[6-[2-cyano-6-fluoro-3-(tetrahydropyran-4-ylsulfonylamino)phenoxy]-4-oxo-quinazolin-3-yl]-7-azaspiro[3.5]nonane-7-carboxylate (3, 400 mg, 493.96 mol, 88% yield) as a light brown gummy liquid. UPLC-MS (ES+): m/z 668.4 [M+H]⁺.

### Step 2

Into a 50 mL single-necked round-bottomed flask containing a well-stirred solution of tert-butyl 2-[6-[2-cyano-6-fluoro-3-(tetrahydropyran-4-ylsulfonylamino)phenoxy]-4-oxo-quinazolin-3-yl]-7-azaspiro[3.5]nonane-7-carboxylate (3, 400 mg, 491.20 mol) in dichloromethane (10 mL) was added trifluoroacetic acid (4.91 mmol, 375.89 L) at 0° C. Reaction mixture was stirred at room temperature for 6 hours. After completion of the starting material, the reaction mixture was concentrated under reduced pressure to get crude residue, which was triturated with methyl tert-butyl ether (2×20 mL) and dried to afford N-[3-[3-(7-azaspiro[3.5]nonan-2-yl)-4-oxo-quinazolin-6-yl]oxy-2-cyano-4-fluoro-phenyl]tetrahydropyran-4-sulfonamide (A-27, 350 mg, 438.03 mol, 89% yield, TFA salt) as a brown semi solid. UPLC-MS (ES+): m/z 568.4 [M+H]⁺.

### Synthesis A28: Synthesis of N-[3-[3-(7-azaspiro[3.5]nonan-2-yl)-4-oxo-quinazolin-6-yl]oxy-2-cyano-4-fluoro-phenyl]-3-methoxy-azetidine-1-sulfonamide (A-28)

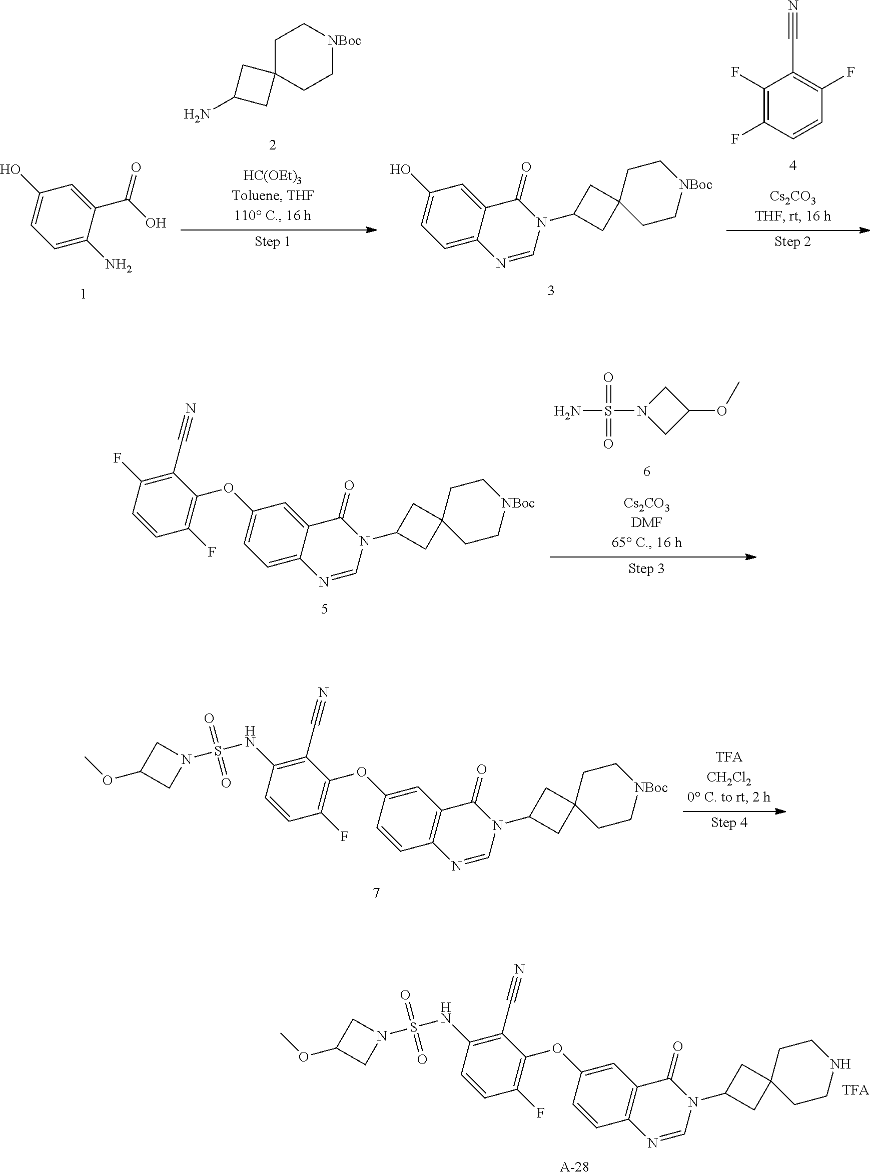

### Step 1

Into a 250 mL three-neck round-bottom flask containing a well stirred solution of 2-amino-5-hydroxy-benzoic acid (1, 15 g, 97.95 mmol), tert-butyl 2-amino-7-azaspiro[3.5]nonane-7-carboxylate (2, 23.54 g, 97.95 mmol) in Toluene (300 mL) and THF (60 mL) was added triethyl orthoformate (146.93 mmol, 24.44 mL) and resulting mixture stirred at 110° C. for 16 hours. After completion of the reaction, the reaction mixture was diluted with water (250 mL) and extracted with ethyl acetate (3×250 mL). The organic layer was washed with NaHCO₃ solution (300 mL), saturated NaCl (250 mL), dried over anhydrous Na₂SO₄ and concentrated under reduced pressure to provide crude residue to afford tert-butyl 2-(6-hydroxy-4-oxo-quinazolin-3-yl)-7-azaspiro[3.5]nonane-7-carboxylate (18.5 g, 41.76 mmol, 43% yield) as a brown solid. UPLC-MS (ES+): m/z 386.1 [M+H]⁺.

### Step 2

Into a 250 mL three-neck round-bottom flask containing a well stirred solution of tert-butyl 2-(6-hydroxy-4-oxo-quinazolin-3-yl)-7-azaspiro[3. 5]nonane-7-carboxylate (3, (18.5 g, 41.76 mmol) in THF (250 mL) were added Cs₂CO₃ (68.02 g, 208.78 mmol) and 2,3,6-trifluorobenzonitrile (4, 50.11 mmol, 5.79 mL) at room temperature under nitrogen atmosphere. The reaction mixture was stirred at same temperature for 16 hours. After completion of the reaction, the reaction mixture was diluted with water (100 mL), extracted with ethyl acetate (2×250 mL). The organic layer was washed with saturated NaCl solution (150 mL), the combined organic layers were dried over anhydrous Na₂SO₄ and concentrated under reduced pressure to get crude residue, which was purified by silica gel column chromatography (100-200 mesh) using a gradient of 0-100% ethyl acetate/hexane to afford tert-butyl 2-[6-(2-cyano-3,6-difluoro-phenoxy)-4-oxo-quinazolin-3-yl]-7-azaspiro[3.5]nonane-7-carboxylate (5, 14.4 g, 27.15 mmol, 65% yield) as an off-white solid. UPLC-MS (ES+): m/z 523.3 [M+H]⁺.

### Step 3

Into a 40 mL pressure vial containing a well stirred solution of tert-butyl 2-[6-(2-cyano-3,6-difluoro-phenoxy)-4-oxo-quinazolin-3-yl]-7-azaspiro[3.5]nonane-7-carboxylate (5, 0.3 g, 565.56 mol) and 3-methoxyazetidine-1-sulfonamide (6, 262.35 mg, 1.25 mmol) in N,N-dimethylformamide (8 mL) was added Cs₂CO₃ (552.81 mg, 1.70 mmol) at room temperature under nitrogen atmosphere. Reaction mixture was stirred at 65° C. for 16 hours. After completion of the reaction, the reaction mixture was quenched with ice cold water (20 mL) and filtered through a celite pad, filtrate was extracted with ethyl acetate (2×20 mL). Combined organic layers were dried over anhydrous Na₂SO₄, concentrated under reduced pressure to afford crude tert-butyl 2-[6-[2-cyano-6-fluoro-3-[(3-methoxyazetidin-1-yl)sulfonylamino]phenoxy]-4-oxo-quinazolin-3-yl]-7-azaspiro[3.5]nonane-7-carboxylate (7, 250 mg, 299.09 mol, 53% yield) as a brown sticky liquid. UPLC-MS (ES+): m/z 669.2 [M+H]⁺.

### Step 4

Into a 25 mL single-neck round-bottom flask containing a well stirred solution of tert-butyl 2-[6-[2-cyano-6-fluoro-3-[(3-methoxyazetidin-1-yl)sulfonylamino]phenoxy]-4-oxo-quinazolin-3-yl]-7-azaspiro[3.5]nonane-7-carboxylate (7, 250 mg, 299.09 mol) in dichloromethane (5 mL) was added trifluoroacetic acid (19.60 mmol, 1.50 mL) at 0° C. under nitrogen atmosphere. The reaction mixture was stirred at room temperature for 2 hours. After completion of the reaction, the reaction mixture was concentrated under reduced pressure and triturated with methyl tert-butyl ether (2×3 mL) to afford N-[3-[3-(7-azaspiro[3.5]nonan-2-yl)-4-oxo-quinazolin-6-yl]oxy-2-cyano-4-fluoro-phenyl]-3-methoxy-azetidine-1-sulfonamide (A-28, 0.24 g, 294.13 mol, 98% yield, TFA salt) as a white solid. UPLC-MS (ES+): m/z 569.2 [M+H]⁺.

### Synthesis A29: Synthesis of (1R,5S)-N-[3-[3-(7-azaspiro[3.5]nonan-2-yl)-4-oxo-quinazolin-6-yl]oxy-2-cyano-4-fluoro-phenyl]-3-azabicyclo[3.1.0]hexane-3-sulfonamide (A-29)

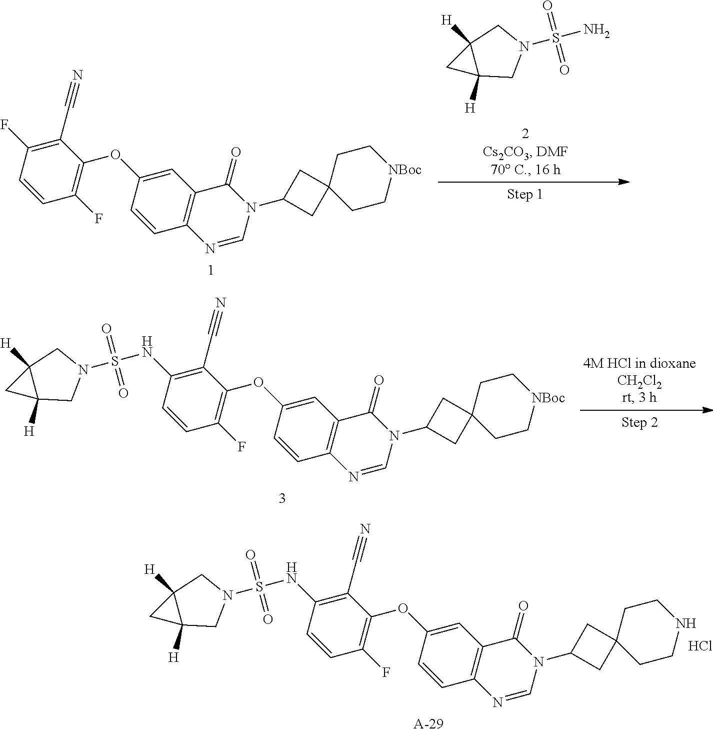

### Step 1

Into a 30 mL glass pressure vial containing a well stirred solution of tert-butyl 2-[6-(2-cyano-3,6-difluoro-phenoxy)-4-oxo-quinazolin-3-yl]-7-azaspiro[3.5]nonane-7-carboxylate (1, 400 mg, 672.86 mol) in N,N-dimethylformamide (3 mL) were added 3-azabicyclo[3.1.0]hexane-3-sulfonamide (2, 330.74 mg, 2.02 mmol) and Cs₂CO₃ (657.70 mg, 2.02 mmol) at room temperature under nitrogen atmosphere. The resulting mixture was stirred at 70° C. for 16 hours. After completion of the reaction, the reaction mixture was concentrated under reduced pressure to get the crude compound, which was purified by column chromatography (25 g, 230-400 silica gel mesh) with 0-100% ethyl acetate in hexane to afford tert-butyl 2-[6-[3-[[(1R,5S)-3-azabicyclo[3.1.0]hexan-3-yl]sulfonylamino]-2-cyano-6-fluoro-phenoxy]-4-oxo-quinazolin-3-yl]-7-azaspiro[3.5]nonane-7-carboxylate (3, 300 mg, 343.48 mol, 51% yield) as an off-white solid. UPLC-MS (ES+): m/z 665.2 [M+H]⁺.

### Step 2

Into a 25 mL single neck round bottom flask containing a well stirred solution of tert-butyl 2-[6-[3-[[(1R,5S)-3-azabicyclo[3.1.0]hexan-3-yl]sulfonylamino]-2-cyano-6-fluoro-phenoxy]-4-oxo-quinazolin-3-yl]-7-azaspiro[3.5]nonane-7-carboxylate (3, 300 mg, 451.30 mol) in anhydrous dichloromethane (10 mL) was added 4M HCl in 1,4-dioxane (4 M, 3 mL) at 0° C. under nitrogen atmosphere. The reaction mixture was stirred at room temperature for 3 hours. After completion of the reaction, the reaction mixture was concentrated under reduced pressure to get a crude residue, which was triturated with methyl tert-butyl ether (2 mL) and dried under vacuum to afford (1R,5S)-N-[3-[3-(7-azaspiro[3.5]nonan-2-yl)-4-oxo-quinazolin-6-yl]oxy-2-cyano-4-fluoro-phenyl]-3-azabicyclo[3.1.0]hexane-3-sulfonamide (A-29, 250 mg, 351.11 mol, 78% yield, HCl salt) as an off-white solid. UPLC-MS (ES+): m/z 565.3 [M+H]⁺.

### Synthesis A30: Synthesis of 3-(2-azaspiro[3.3]heptan-6-yl)-6-[2-cyano-3-[[ethyl(methyl)sulfamoyl]amino]-6-fluoro-phenoxy]-4-oxo-quinazoline (A-30)

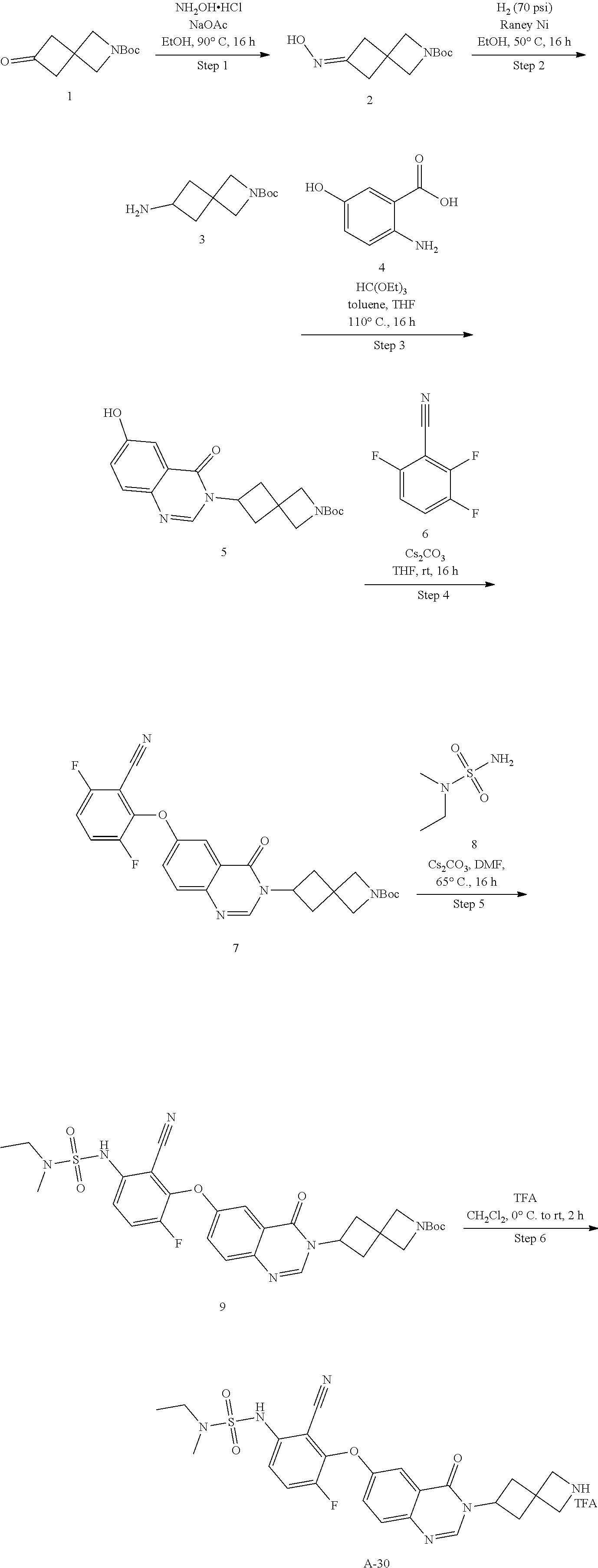

### Step 1

Into a 100 mL two-neck round-bottom flask containing a well stirred solution of tert-butyl 6-oxo-2-azaspiro[3.3]heptane-2-carboxylate (1, 1 g, 4.73 mmol) in ethanol (20 mL) were added hydroxylamine hydrochloride (493.40 mg, 7.10 mmol, 295.45 L) and NaOAc (970.74 mg, 11.83 mmol, 635.30 μL) at room temperature under nitrogen atmosphere. The resulting suspension was stirred at 90° C. for 16 hours. After completion of the reaction, the reaction mixture was allowed to reach room temperature, diluted with water (20 mL) and ethyl acetate (60 mL). The layers were separated, and the aqueous layer was extracted with ethyl acetate (2×50 mL). Combined organic layers were washed with saturated NaCl solution (40 mL), dried over anhydrous Na₂SO₄, filtered and filtrate was concentrated under reduced pressure to afford tert-butyl 6-hydroxyimino-2-azaspiro[3.3]heptane-2-carboxylate (2, 850 mg, 3.68 mmol, 78% yield) as an off-white solid. UPLC-MS (ES+): m/z 171.3 [M-tBu+H]⁺.

### Step 2

A 100 mL tinyclave glass pressure flask containing a well stirred solution of tert-butyl 6-hydroxyimino-2-azaspiro[3.3]heptane-2-carboxylate (2, 850 mg, 3.76 mmol) in EtOH (15 mL) was degassed with nitrogen for 10 min. Raney Ni in water (643.68 mg, 7.51 mmol) was added to the reaction mixture and stirred under hydrogen (5 kg/cm²) pressure at 50° C. for 16 hours. After completion of the reaction, the reaction mixture was filtered through a celite bed and washed with ethyl acetate (150 mL). Filtrate was concentrated under reduced pressure to afford tert-butyl 6-amino-2-azaspiro[3.3]heptane-2-carboxylate (3, 710 mg, 1.94 mmol, 52% yield) as an off-white gum. The desired product was not isolated and taken to the next step without purification. UPLC-MS (ES+): m/z 213.4 [M+H]⁺.

### Step 3

Into a 40 mL pressure vial containing a well stirred solution of 2-amino-5-hydroxybenzoic acid (4, 250 mg, 1.63 mmol) and tert-butyl 6-amino-2-azaspiro[3.3]heptane-2-carboxylate (3, 596.20 mg, 1.63 mmol) in toluene (7 mL) and THF (3 mL) was added triethyl orthoformate (3.59 mmol, 597.39 L) at room temperature and stirred at 110° C. for 16 hours. After completion of the reaction, the reaction mixture was allowed to room temperature and diluted with water (20 mL) and ethyl acetate (30 mL). The layers were separated, and aqueous layer was extracted with ethyl acetate (2×50 mL). Combined organic layers were dried over anhydrous Na₂SO₄, filtered and filtrate was concentrated under reduced pressure to get crude which was purified by flash silica-gel (230-400 mesh) column with 0-100% ethyl acetate/hexane to afford tert-butyl 6-(6-hydroxy-4-oxo-quinazolin-3-yl)-2-azaspiro[3.3]heptane-2-carboxylate (5, 410 mg, 1.02 mmol, 62% yield) as a brown gum. UPLC-MS (ES+): m/z 358.3 [M+H]⁺.

### Step 4

Into a 40 mL pressure vial containing a well-stirred solution of tert-butyl 6-(6-hydroxy-4-oxo-quinazolin-3-yl)-2-azaspiro[3.3]heptane-2-carboxylate (5, 410 mg, 1.01 mmol) in THF (8 mL) were added 2,3,6-trifluorobenzonitrile (6, 190.30 mg, 1.21 mmol, 139.93 L) and Cs₂CO₃ (986.75 mg, 3.03 mmol) at room temperature under nitrogen atmosphere. The reaction mixture was stirred at room temperature for 16 hours. After completion of the reaction, the reaction mixture was diluted with water (20 mL) and ethyl acetate (40 mL). The layers were separated, and aqueous layer was extracted with ethyl acetate (2×20 mL). Combined organic layers were washed with saturated NaCl (30 mL), dried over anhydrous Na₂SO₄, filtered and filtrate was concentrated under reduced pressure to get crude which was purified by flash silica-gel (230-400 mesh) column with 0-100% ethyl acetate/hexane to afford tert-butyl 6-[6-(2-cyano-3,6-difluoro-phenoxy)-4-oxo-quinazolin-3-yl]-2-azaspiro[3.3]heptane-2-carboxylate (7, 400 mg, 692.92 [tmol, 69% yield) as an off-white solid. UPLC-MS (ES+): m/z 495.3 [M+H]⁺.

### Step 5

Into a 40 mL pressure vial containing a well-stirred solution of tert-butyl 6-[6-(2-cyano-3,6-difluoro-phenoxy)-4-oxo-quinazolin-3-yl]-2-azaspiro[3.3]heptane-2-carboxylate (7, 400 mg, 692.43 mol) in N,N-dimethylformamide (10 mL) were added [methyl(sulfamoyl)amino]ethane (8, 143.53 mg, 1.04 mmol) and Cs₂CO₃ (676.82 mg, 2.08 mmol) at room temperature and stirred at 65° C. for 16 hours. After completion of the reaction, the reaction mixture was diluted with water (20 mL) and filtered through Buchner funnel to remove the solid fluorescent impurity. The filtrate was extracted with ethyl acetate (2×30 mL) and combined organic layers were washed with cold water (30 mL), dried over anhydrous Na₂SO₄, filtered and concentrated under reduced pressure to afford tert-butyl 6-[6-[2-cyano-3-[[ethyl(methyl)sulfamoyl]amino]-6-fluoro-phenoxy]-4-oxo-quinazolin-3-yl]-2-azaspiro[3.3]heptane-2-carboxylate (9, 390 mg, 490.53 mol, 71% yield) as a brown gum. UPLC-MS (ES+): m/z 613.2 [M+H]⁺.

### Step 6

Into a 25 mL single-neck round-bottom flask containing a well-stirred solution of tert-butyl 6-[6-[2-cyano-3-[[ethyl(methyl)sulfamoyl]amino]-6-fluoro-phenoxy]-4-oxo-quinazolin-3-yl]-2-azaspiro[3.3]heptane-2-carboxylate (9, 390 mg, 490.53 mol) in dichloromethane (6 mL) was added trifluoroacetic acid (19.64 mmol, 1.50 mL) slowly at 0° C. under nitrogen atmosphere. The reaction mixture was stirred at room temperature for 2 hours. After completion of the reaction, the reaction mixture was concentrated under reduced pressure to obtain the crude residue which was triturated with methyl tert-butyl ether (2×3 mL), decanted and dried under reduced pressure to afford 3-(2-azaspiro[3.3]heptan-6-yl)-6-[2-cyano-3-[[ethyl(methyl)sulfamoyl]amino]-6-fluoro-phenoxy]-4-oxo-quinazoline (A-30, 350 mg, 476.53 mol, 97% yield, TFA salt) as a brown gum. UPLC-MS (ES+): m/z 513.3 [M+H]⁺.

### Synthesis A31: Synthesis of 3-(6-azaspiro[3.4]octan-2-yl)-6-[2-cyano-3-[[ethyl(methyl)sulfamoyl]amino]-6-fluoro-phenoxy]-4-oxo-quinazoline (A-31)

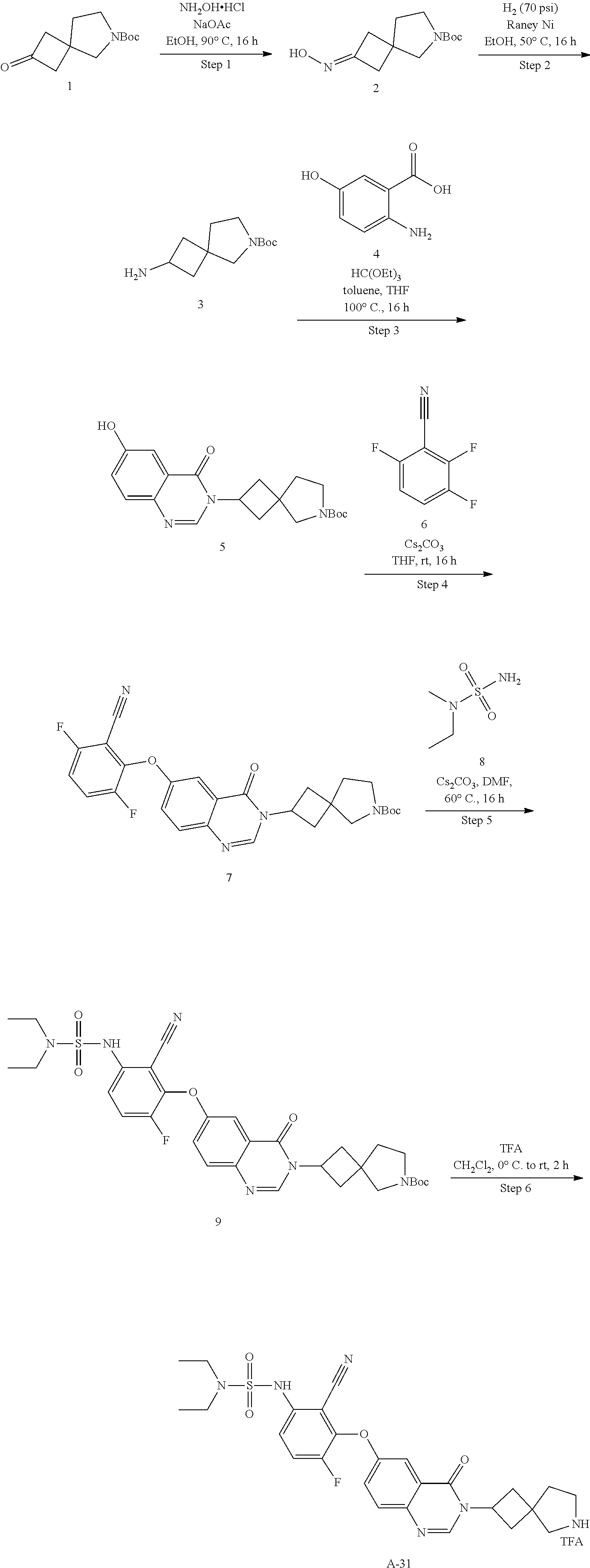

### Step 1

Into a 100 mL two-neck round-bottom flask containing a well stirred solution of tert-butyl 2-oxo-6-azaspiro[3.4]octane-6-carboxylate (1, 1.00 g, 4.44 mmol) in ethanol (20 mL) were added NaOAc (910.30 mg, 11.10 mmol, 595.74 L) and hydroxylamine hydrochloride (462.68 mg, 6.66 mmol, 277.06 μL) at room temperature under nitrogen atmosphere. Resulting reaction suspension was stirred at 90° C. for 16 hours. After completion of the reaction, the reaction mixture was allowed to reach room temperature, diluted with water (20 mL) and ethyl acetate (60 mL). The layers were separated, and aqueous layer was extracted with ethyl acetate (2×60 mL). Combined organic layers were washed with saturated NaCl solution (40 mL), dried over anhydrous Na₂SO₄, filtered and filtrate was concentrated under reduced pressure to afford tert-butyl 2-hydroxyimino-6-azaspiro[3.4]octane-6-carboxylate (2, 0.98 g, 4.05 mmol, 91% yield) as an off-white solid. UPLC-MS (ES+): m/z 185.2 [M+H−^{t}Bu]⁺.

### Step 2

A 100 mL tinyclave glass pressure flask containing a well stirred solution of tert-butyl 2-hydroxyimino-6-azaspiro[3.4]octane-6-carboxylate (2, 1.20 g, 4.94 mmol) in EtOH (20 mL) was degassed with nitrogen for 10 min. Raney Ni in water (847.13 mg, 9.89 mmol) was added to the reaction mixture and stirred under hydrogen (5 kg/cm²) pressure at 50° C. for 16 hours. After completion of the reaction, the reaction mixture was filtered through a celite bed and washed with ethyl acetate (300 mL). Filtrate was concentrated under reduced pressure to afford tert-butyl 2-amino-6-azaspiro[3.4]octane-6-carboxylate (3, 0.95 g, 1.65 mmol, 33% yield) as an off-white gum. Crude product was taken to the next step without purification. UPLC-MS (ES+): m/z 171.0 [M+H−^{t}Bu]⁺.

### Step 3

Into a 40 mL pressure vial containing a well stirred solution tert-butyl 2-amino-6-azaspiro[3.4]octane-6-carboxylate (3, 900.00 mg, 3.98 mmol) and 2-amino-5-hydroxy-benzoic acid (4, 608.98 mg, 3.98 mmol) in THF (4 mL) and toluene (16 mL) was added triethyl orthoformate (5.97 mmol, 992.18 L) at room temperature and the resulting suspension was stirred at 100° C. for 16 hours. After completion of the reaction, the reaction mixture was allowed to reach room temperature and diluted with water (30 mL) and ethyl acetate (30 mL). The layers were separated, and aqueous layer was extracted with ethyl acetate (2×60 mL). Combined organic layers were dried over anhydrous Na₂SO₄, filtered and filtrate was concentrated under reduced pressure to get crude which was purified by flash silica-gel (230-400 mesh) column with 0-100% ethyl acetate/hexane to afford tert-butyl 2-(6-hydroxy-4-oxo-quinazolin-3-yl)-6-azaspiro[3.4]octane-6-carboxylate (5, 1.0 g, 2.18 mmol, 55% yield) as a pale brown solid. UPLC-MS (ES+): m/z 372.3 [M+H]⁺.

### Step 4

Into a 40 mL pressure vial containing a well-stirred solution of tert-butyl 2-(6-hydroxy-4-oxo-quinazolin-3-yl)-6-azaspiro[3.4]octane-6-carboxylate (5, 0.95 g, 2.07 mmol) in THF (15 mL) were added 2,3,6-trifluorobenzonitrile (6, 390.54 mg, 2.49 mmol, 287.16 L) and Cs₂CO₃ (2.03 g, 6.22 mmol) at room temperature under nitrogen atmosphere. Reaction mixture was stirred at room temperature for 14 hours. After completion of the reaction, the reaction mixture was diluted with water (30 mL) and ethyl acetate (60 mL). The layers were separated, and aqueous layer was extracted with ethyl acetate (2×40 mL). Combined organic layers were washed with saturated NaCl (30 mL), dried over anhydrous Na₂SO₄, filtered and filtrate was concentrated under reduced pressure to get crude which was purified by flash silica-gel (230-400 mesh) column with 0-100% ethyl acetate/hexane to afford tert-butyl 2-[6-(2-cyano-3,6-difluoro-phenoxy)-4-oxo-quinazolin-3-yl]-6-azaspiro[3.4]octane-6-carboxylate (7, 0.5 g, 971.75 mol, 47% yield) as an off-white solid. UPLC-MS (ES+): m/z 509.3 [M+H]⁺.

### Step 5

Into a 40 mL pressure vial containing a well-stirred solution of tert-butyl 2-[6-(2-cyano-3,6-difluoro-phenoxy)-4-oxo-quinazolin-3-yl]-6-azaspiro[3.4]octane-6-carboxylate (7, 500.00 mg, 963.59 mol) in N,N-dimethylformamide (10 mL) were added [methyl(sulfamoyl)amino]ethane (8, 199.74 mg, 1.45 mmol) and Cs₂CO₃ (470.93 mg, 1.45 mmol) at room temperature and stirred at 60° C. for 16 hours. After completion of the reaction, the reaction mixture was diluted with water (20 mL) and filtered through a Buchner funnel to remove the fluorescent impurity. The filtrate was extracted with ethyl acetate (2×20 mL) and the combined organic layers were washed with cold water (20 mL), dried over anhydrous Na₂SO₄, filtered and concentrated under reduced pressure to afford tert-butyl 2-[6-[2-cyano-3-[[ethyl(methyl)sulfamoyl]amino]-6-fluoro-phenoxy]-4-oxo-quinazolin-3-yl]-6-azaspiro[3.4]octane-6-carboxylate (9, 0.3 g, 416.28 mol, 43% yield) as a brown gummy liquid. UPLC-MS (ES+): m/z 627.3 [M+H]⁺.

### Step 6

Into a 50 mL single-neck round-bottom flask containing a well-stirred solution of tert-butyl 2-[6-[2-cyano-3-[[ethyl(methyl)sulfamoyl]amino]-6-fluoro-phenoxy]-4-oxo-quinazolin-3-yl]-6-azaspiro[3.4]octane-6-carboxylate (9, 0.3 g, 415.99 mol) in dichloromethane (6 mL) was added trifluoroacetic acid (11.76 mmol, 0.9 mL) slowly at 0° C. under nitrogen atmosphere. The resulting solution was stirred at room temperature for 2 hours. After completion of the reaction, the reaction mixture was concentrated under reduced pressure and the obtained residue was triturated with methyl tert-butyl ether (2×3 mL), decanted and dried under reduced pressure to afford 3-(6-azaspiro[3.4]octan-2-yl)-6-[2-cyano-3-[[ethyl(methyl)sulfamoyl]amino]-6-fluoro-phenoxy]-4-oxo-quinazoline (A-31, 0.3 g, 414.26 mol, 99% yield, TFA salt) as a brown gummy liquid. UPLC-MS (ES+): m/z 527.3 [M+H]⁺.

### Synthesis A32: Synthesis of (7R)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-32)

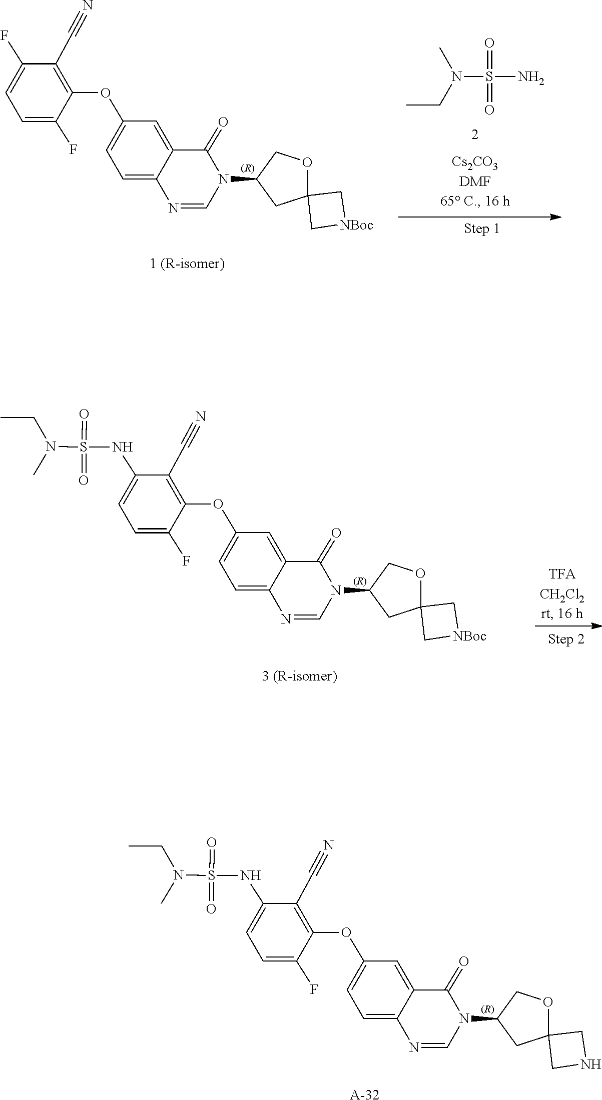

### Step 1

Into a 100 mL double-neck round-bottom flask containing a well-stirred solution of tert-butyl (7R)-7-[6-(2-cyano-3,6-difluoro-phenoxy)-4-oxo-quinazolin-3-yl]-5-oxa-2-azaspiro[3.4]octane-2-carboxylate (1, 900 mg, 1.76 mmol; see intermediate 10b in the synthesis of A³³) and [methyl(sulfamoyl)amino]ethane (2, 487.26 mg, 3.53 mmol) in N,N-dimethylformamide (10 mL) was added Cs₂CO₃ (1.72 g, 5.29 mmol) at room temperature under nitrogen atmosphere. After completion of the reaction, the reaction mixture was stirred at 65° C. for 16 hours. After completion of the reaction, the reaction mixture was diluted with water (30 mL), solid precipitate was filtered, and the filtrate was extracted with ethyl acetate (2×300 mL). Combined organic layer was washed with saturated NaCl (50 mL), dried over anhydrous Na₂SO₄, filtered and filtrate was concentrated under reduced pressure to obtain a crude residue which was purified by flash (230-400 mesh, 100 g) column with 0-100% ethyl acetate/hexane as an eluent to afford tert-butyl (7R)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane-2-carboxylate (3, 500 mg, 723.75 mol, 41% yield) as a brown thick liquid. UPLC-MS (ES−): m/z 627.3 [M−H]⁻.

### Step 2

Into a 100 mL single-neck round-bottom flask containing a well-stirred solution of tert-butyl (7R)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane-2-carboxylate (3, 500 mg, 795.33 mol) in dichloromethane (10 mL) was added trifluoroacetic acid (7.95 mmol, 608.63 L) at 0° C. under nitrogen atmosphere. Resulting reaction mixture was stirred at room temperature for 16 hours. After completion of the reaction, the reaction mixture was concentrated under reduced pressure to get a crude residue which was triturated with n-pentane (2×100 mL) and dried under reduced pressure to afford (7R)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-32, 500 mg, 692.52 mol, 87% yield, TFA salt) as a brown gum. UPLC-MS (ES+): m/z 529.2 [M+H]⁺

### Synthesis A33: Synthesis of (7S)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-33)

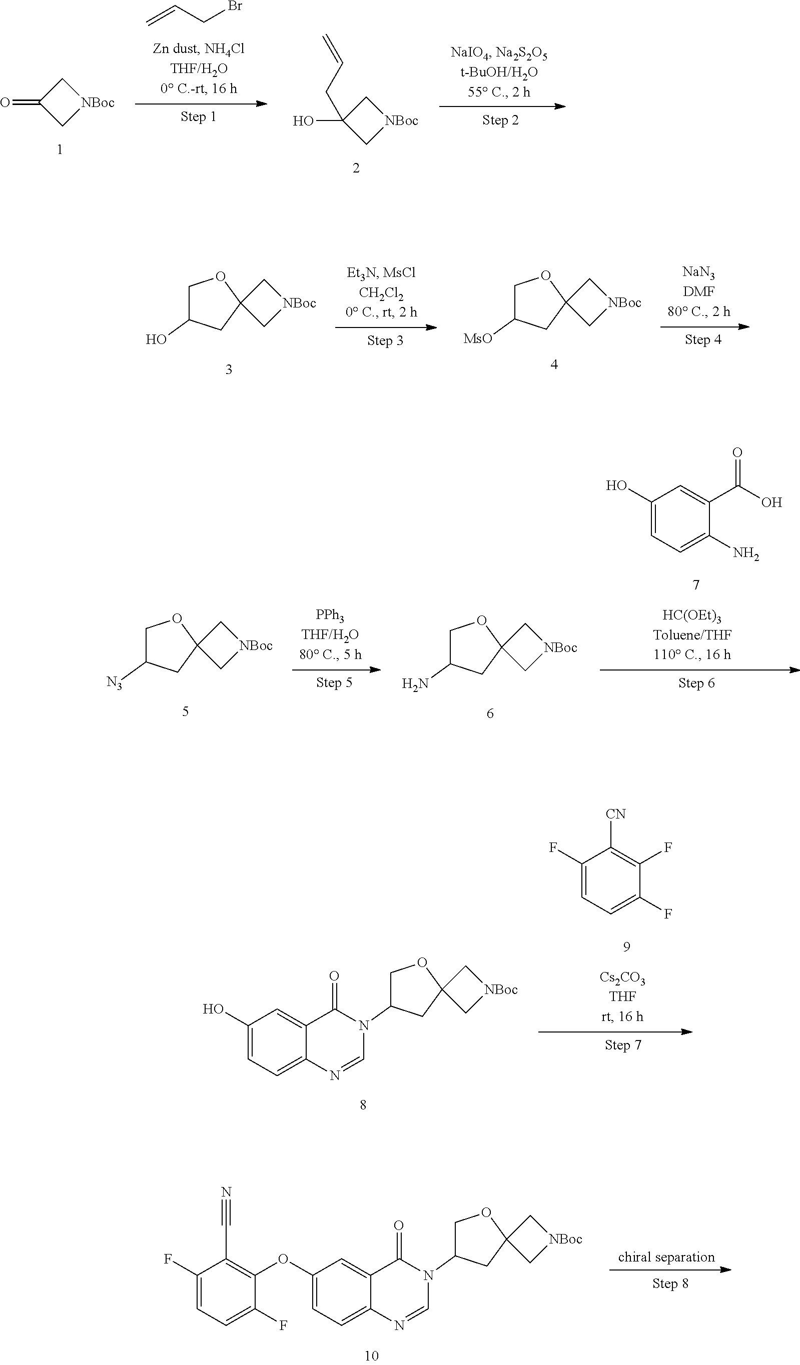

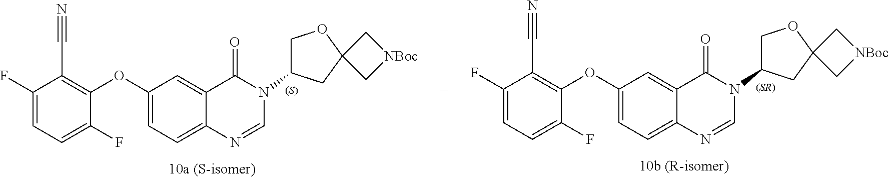

### Step 1

Into a 500 mL double-neck round bottom flask containing a well stirred solution of tert-butyl 3-oxoazetidine-1-carboxylate (1, 15 g, 87.62 mmol) and 3-bromoprop-1-ene (26.50 g, 219.05 mmol, 18.93 mL) in THF (150 mL) and water were added ammonium chloride (300 mL) and zinc dust (11.46 g, 175.24 mmol) at room temperature. The resulting reaction mixture was stirred at room temperature for 16 hours. After completion of the reaction, the reaction mixture was filtered through a celite pad and washed with ethyl acetate (200 mL), dried over anhydrous Na₂SO₄, filtered and concentrated under reduced pressure to afford tert-butyl 3-allyl-3-hydroxy-azetidine-1-carboxylate (2, 18 g, 75.96 mmol, 87% yield) as a colorless liquid. UPLC-MS (ES−): m/z 212.2 [M−H]⁻.

### Step 2

Into a 500 mL double-neck round bottom flask containing a well stirred solution of tert-butyl 3-allyl-3-hydroxy-azetidine-1-carboxylate (2, 10 g, 46.89 mmol) in t-BuOH (200 mL) and water (100 mL) were added sodium metaperiodate (20.06 g, 93.78 mmol) and a solution of sodium metabisulfite (17.83 g, 93.78 mmol) dissolved in water (50 mL) at 0° C. Resulting reaction mixture was stirred at 55° C. for 2 hours. After completion of the reaction, the reaction mixture was diluted with water (200 mL) and extracted with ethyl acetate (2×500 mL). Combined organic layer was dried over anhydrous Na₂SO₄, filtered and concentrated under reduced pressure to afford a crude residue which was purified by flash (silica gel, 230-400 mesh, 220 g) column chromatography with 0-100% ethyl acetate/hexane as an eluent to afford tert-butyl 7-hydroxy-5-oxa-2-azaspiro[3.4]octane-2-carboxylate (3, 4 g, 14.48 mmol, 31% yield) as a brown gum. UPLC-MS (ES+): m/z 130.2 [M+H-Boc]⁺

### Step 3

Into a 250 mL double-neck round-bottom flask containing a well-stirred solution of tert-butyl 7-hydroxy-5-oxa-2-azaspiro[3.4]octane-2-carboxylate (3, 8 g, 34.89 mmol) in dichloromethane (150 mL) were added triethylamine (104.68 mmol, 14.59 mL) and methane sulfonyl chloride (52.34 mmol, 4.06 mL) at 0° C. under nitrogen atmosphere. Resulting reaction mixture was stirred at room temperature for 2 hours. After completion of the reaction, the reaction mixture was quenched with water (50 mL) and extracted with dichloromethane (2×300 mL). Combined organic layer was dried over anhydrous Na₂SO₄, filtered and concentrated under reduced pressure to afford tert-butyl 7-methylsulfonyloxy-5-oxa-2-azaspiro[3.4]octane-2-carboxylate (4, 8 g, 21.60 mmol, 62% yield) as a brown gum. UPLC-MS (ES+): m/z 208.0 [M+H-Boc]⁺.

### Step 4

Into a 250 mL double-neck round-bottom flask containing a well-stirred solution of tert-butyl 7-methylsulfonyloxy-5-oxa-2-azaspiro[3.4]octane-2-carboxylate (4, 8 g, 26.03 mmol) in anhydrous N,N-dimethylformamide (100 mL) was added sodium azide (2.54 g, 39.04 mmol) at room temperature under nitrogen atmosphere. Resulting reaction mixture was stirred at 80° C. for 2 hours. After completion of the reaction, the reaction mixture was quenched with water (100 mL) and extracted with ethyl acetate (2×400 mL). Combined organic layer was dried over anhydrous Na₂SO₄, filtered and concentrated under reduced pressure to afford tert-butyl 7-azido-5-oxa-2-azaspiro[3.4]octane-2-carboxylate (5, 8 g, 25.14 mmol, 97% yield) as a colorless liquid. UPLC-MS (ES+): m/z 155.2 [M+H-Boc]⁺.

### Step 5

Into a 250 mL double-neck round-bottom flask containing a well stirred solution of tert-butyl 7-azido-5-oxa-2-azaspiro[3.4]octane-2-carboxylate (5, 8 g, 31.46 mmol) in THF (120 mL) and water (40 mL) was added triphenylphosphine (12.38 g, 47.19 mmol) at room temperature. Resulting reaction mixture was stirred at 80° C. for 5 hours. After completion of the reaction, the reaction mixture was quenched with water (50 mL) and extracted with ethyl acetate (3×150 mL). Combined organic layer was dried over anhydrous Na₂SO₄, filtered and concentrated under reduced pressure to get crude residue which was purified by flash (230-400 mesh, 100 g) column with 0-20% methanol/dichloromethane as an eluent to afford tert-butyl 7-amino-5-oxa-2-azaspiro[3.4]octane-2-carboxylate (6, 2 g, 8.50 mmol, 27% yield) as a pale yellow gummy liquid. UPLC-MS (ES+): m/z 229.2 [M+H]⁺.

### Step 6

Into a 250 mL double-neck round-bottom flask containing a well-stirred solution of tert-butyl 7-amino-5-oxa-2-azaspiro[3.4]octane-2-carboxylate (6, 2.5 g, 10.95 mmol), 2-amino-5-hydroxy-benzoic acid (7, 1.68 g, 10.95 mmol) in toluene (39.99 mL) and THF (10 mL) was added triethyl orthoformate (16.43 mmol, 2.73 mL) at room temperature. Resulting reaction mixture was stirred at 110° C. for 16 hours. After completion of the reaction, the reaction mixture was diluted with water (100 mL) and extracted with ethyl acetate (2×400 mL). Combined organic layer was washed with NaHCO₃ solution (50 mL), dried over anhydrous Na₂SO₄ and concentrated under reduced pressure to afford a crude residue which was purified by flash (230-400 silica mesh, 100 g) column with 0-100% ethyl acetate/hexane as an eluent to afford tert-butyl 7-(6-hydroxy-4-oxo-quinazolin-3-yl)-5-oxa-2-azaspiro[3.4]octane-2-carboxylate (8, 2 g, 5.03 mmol, 46% yield) as an off-white solid. UPLC-MS (ES+): m/z 374.2 [M+H]⁺.

### Step 7

Into a 100 mL double-neck round bottom flask containing a well stirred solution of tert-butyl 7-(6-hydroxy-4-oxo-quinazolin-3-yl)-5-oxa-2-azaspiro[3.4]octane-2-carboxylate (8, 2 g, 5.36 mmol) in THF (20 mL) were added Cs₂CO₃ (5.24 g, 16.07 mmol) and 2,3,6-trifluorobenzonitrile (9, 6.43 mmol, 742.42 L) at room temperature. Resulting reaction mixture was stirred at room temperature for 16 hours. After completion of the reaction, the reaction mixture was diluted with water (40 mL) and extracted with ethyl acetate (2×200 mL). Combined organic layer was washed with saturated NaCl solution (40 mL), dried over anhydrous Na₂SO₄ and filtered, concentrated under reduced pressure to get a crude residue which was purified by flash (230-400 mesh, 100 g) column chromatography using 0-100% ethyl acetate/hexane as an eluent to afford tert-butyl 7-(6-(2-cyano-3,6-difluorophenoxy)-4-oxoquinazolin-3(4H)-yl)-5-oxa-2-azaspiro[3.4]octane-2-carboxylate (10, 2.5 g) as an off-white solid. UPLC-MS (ES+): m/z 511.2 [M+H]⁺.

Step 8: Chiral SFC separation: Above isomeric mixture of tert-butyl 7-(6-(2-cyano-3,6-difluorophenoxy)-4-oxoquinazolin-3(4H)-yl)-5-oxa-2-azaspiro[3.4]octane-2-carboxylate (10, 2.5 g) was purified by chiral SFC following the below method.

### Method of Analysis for Chiral SFC Separation

Instrument: PIC 22-027
Column: I CELLULOSE-Z-(250 mm*20 mm, 5 m)
Mobile Phase: CO₂: (0.5% isopropyl amine in methanol)_[55:45]
Total Flow: 60 ml/min
Back pressure: 110 bar
Wavelength: 254 nm
Cycle time: 7.0 min

Fast eluting isomer (Peak-1): Fast eluting compound (Peak-1, t_{R}=2.21 min) was collected and concentrated under reduced pressure to afford tert-butyl (7S)-7-[6-(2-cyano-3,6-difluoro-phenoxy)-4-oxo-quinazolin-3-yl]-5-oxa-2-azaspiro[3.4]octane-2-carboxylate (10a 900 mg, 1.75 mmol, 33% yield) as an off-white solid. Peak-1 was designated as an S-isomer based on preliminary data. UPLC-MS (ES+): m/z 511.2 [M+H]⁺.

Late eluting isomer (Peak-2): Late eluting compound (Peak-2, t_{R}=2.63 min) was collected and concentrated under reduced pressure to afford tert-butyl (7R)-7-[6-(2-cyano-3,6-difluoro-phenoxy)-4-oxo-quinazolin-3-yl]-5-oxa-2-azaspiro[3.4]octane-2-carboxylate (10b, 900 mg, 1.76 mmol, 33% yield) as an off-white solid. Peak-2 was designated as an R-isomer based on preliminary data. UPLC-MS (ES+): m/z 511.2 [M+H]⁺.

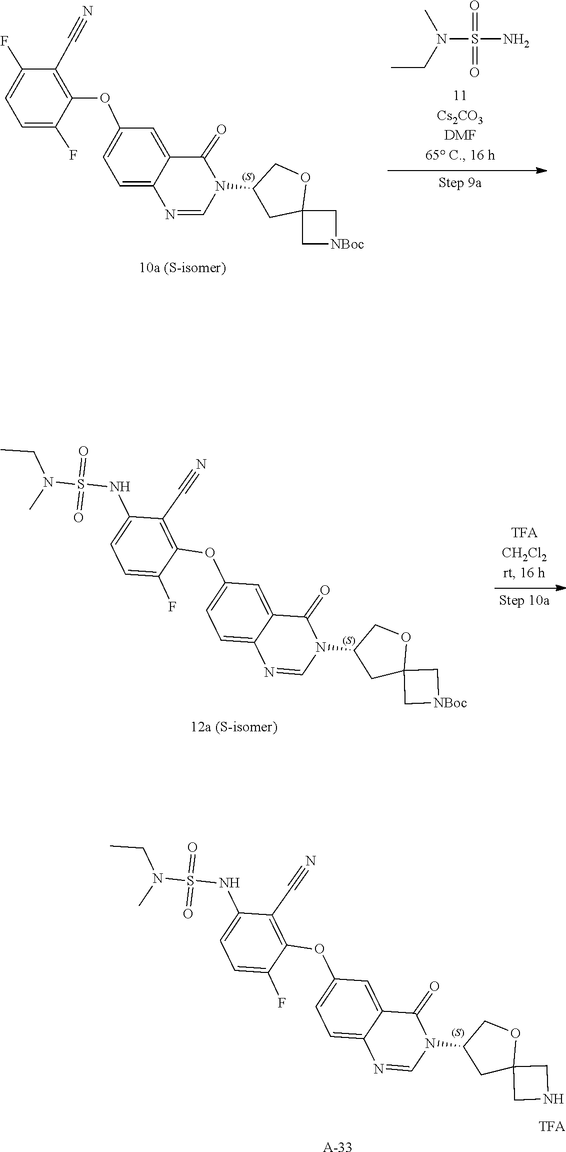

### Step 9a

Into a 100 mL double-neck round-bottom flask containing a well-stirred solution of tert-butyl (7S)-7-[6-(2-cyano-3,6-difluoro-phenoxy)-4-oxo-quinazolin-3-yl]-5-oxa-2-azaspiro[3.4]octane-2-carboxylate (10a, 900 mg, 1.76 mmol) and [methyl(sulfamoyl)amino]ethane (11, 487.26 mg, 3.53 mmol) in N,N-dimethylformamide (10 mL) was added Cs₂CO₃ (1.72 g, 5.29 mmol) at room temperature under nitrogen atmosphere and stirred at 65° C. for 16 hours. After completion of the reaction, the reaction mixture was diluted with water (30 mL), solid precipitate was filtered through Celite pad and filtrate was extracted with ethyl acetate (2×300 mL). Combined organic layer was washed with saturated NaCl (50 mL), dried over anhydrous Na₂SO₄, filtered and concentrated under reduced pressure to afford a crude residue which was purified by flash (230-400 mesh, 100 g) column chromatography using 0-100% ethyl acetate/hexane as an eluent to afford tert-butyl (7S)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane-2-carboxylate (12a, 600 mg, 944.85 mol, 54% yield) as a brown thick liquid. UPLC-MS (ES−): m/z 627.0 [M−H]⁻.

### Step 10a

Into a 100 mL single-neck round-bottom flask containing a well-stirred solution of tert-butyl (7S)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane-2-carboxylate (12a, 600 mg, 954.40 mol) in dichloromethane (10 mL) was added trifluoroacetic acid (39.20 mmol, 3.00 mL) at 0° C. under nitrogen atmosphere and resulting reaction mixture was stirred at the room temperature for 16 hours. After completion of the reaction, the reaction mixture was concentrated under reduced pressure to get a crude residue which was triturated with n-pentane (2×100 mL) and dried under reduced pressure to afford (7S)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-33, 600 mg, 662.95 mol, 69% yield, TFA salt) as a brown gum. UPLC-MS (ES+): m/z 529.2 [M+H]⁺.

### Synthesis A34: Synthesis of 3-(7-azaspiro[3.5]nonan-2-yl)-6-[3-[[ethyl(methyl)sulfamoyl]amino]-2,6-difluoro-benzoyl]-4-oxo-quinazoline (A-34)

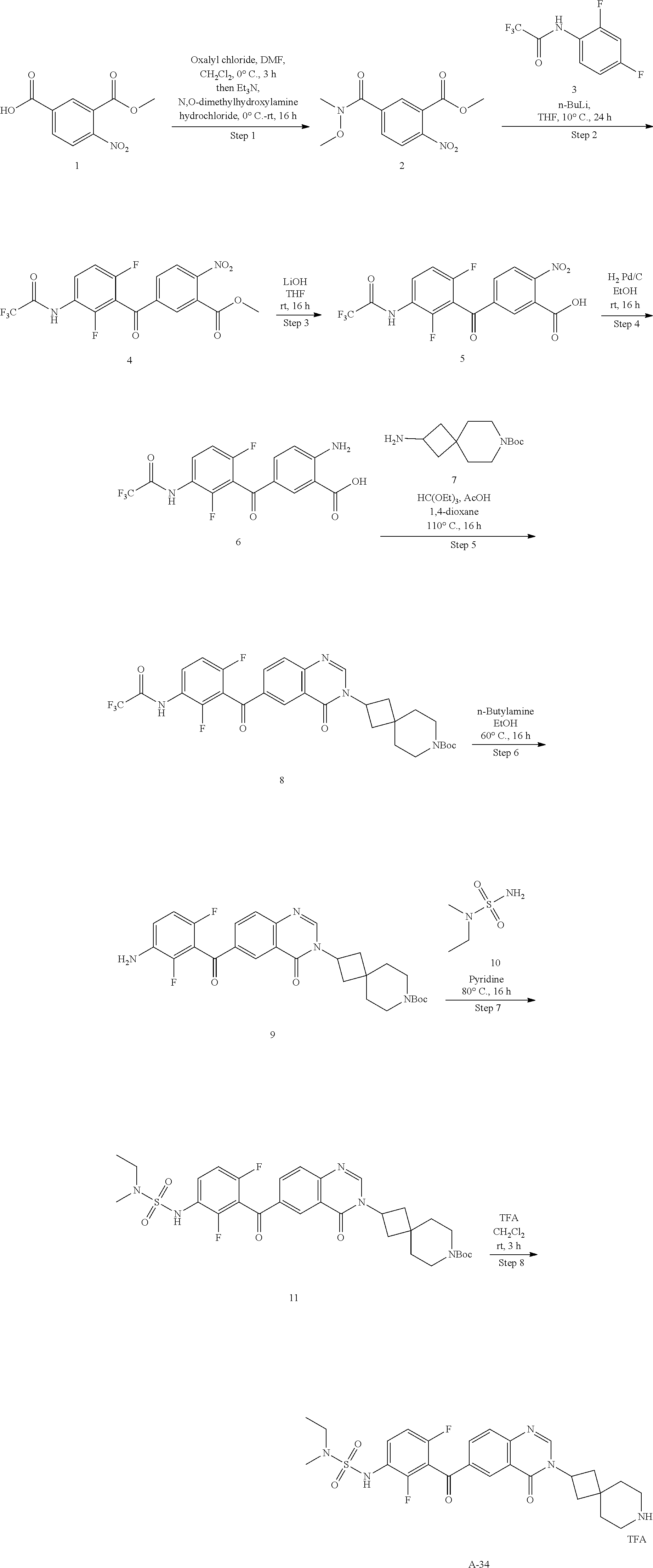

### Step 1

Into a 1000 mL three-neck round bottom flask containing a well-stirred solution of 3-methoxycarbonyl-4-nitro-benzoic acid (1, 10 g, 44.41 mmol) in dry dichloromethane (150 mL) was added oxalyl chloride (53.30 mmol, 4.65 mL) followed by N,N-dimethylformamide (3.25 mg, 44.41 mol, 3.44 L) at 0° C. for 3 hours under nitrogen atmosphere. The reaction mixture was stirred at room temperature for 3 hours and a clear solution was observed which indicated the formation of acid chloride. At this point of time, reaction mixture was again cooled to 0° C. and was added triethylamine (13.48 g, 133.24 mmol, 18.57 mL) slowly over a period of 15 minutes followed by addition of N,O-dimethylhydroxylamine hydrochloride (4.55 g, 46.63 mmol). The reaction mixture was stirred at room temperature for 16 hours. The reaction mixture was poured into ice-cold water (350 mL) and extracted with dichloromethane (2×250 mL). Organic phases were combined and washed with saturated NaCl (100 mL), dried over anhydrous Na₂SO₄, filtered and concentrated under reduced pressure to get a crude residue which was purified by flash silica-gel (230-400 mesh) column using 0-100% ethyl acetate/petroleum ether as an eluent to afford methyl 5-[methoxy(methyl)carbamoyl]-2-nitro-benzoate (2, 10 g, 36.37 mmol, 82% yield) as a brown liquid. UPLC-MS (ES+): m/z 269.0 [M+H]⁺.

### Step 2

Into a 250 mL three-neck round bottom flask containing a well stirred solution of N-(2,4-difluorophenyl)-2,2,2-trifluoro-acetamide (3, 4 g, 17.77 mmol) in anhydrous THF (60 mL) was cooled to −78° C. and added n-BuLi (2.5 M, 15.64 mL) over a period of 10 minutes. The reaction mixture was stirred at −78° C. for 1 hours. To the above mixture methyl 5-[methoxy(methyl)carbamoyl]-2-nitro-benzoate (2, 5.00 g, 18.66 mmol) dissolved in THF (10 mL) was added quickly. The reaction mixture was stirred for 2 h at −78° C. After completion of the reaction, the reaction mixture was quenched with saturated aqueous NH₄C₁ solution (100 mL) and extracted with ethyl acetate (2×100 mL). The combined organics were dried over anhydrous Na₂SO₄, filtered and concentrated under reduced pressure to get a crude residue which was purified by flash silica-gel (230-400 mesh; 100 g) column chromatography using 0-100% ethyl acetate in petroleum ether as an eluent to afford methyl 5-[2,6-difluoro-3-[(2,2,2-trifluoroacetyl)amino]benzoyl]-2-nitro-benzoate (4, 5.3 g, 11.94 mmol, 67% yield) as a gummy liquid. UPLC-MS (ES+): m/z 431.1 [M+H]⁺.

### Step 3

Into a 250 mL single-neck round-bottom flask containing a well stirred solution of methyl 5-[2,6-difluoro-3-[(2,2,2-trifluoroacetyl) amino]benzoyl]-2-nitro-benzoate (4, 5 g, 11.57 mmol) in water (10 mL) and THF (75 mL) was added LiOH·H₂O (582.43 mg, 13.88 mmol, 12.72 mL) at 0° C. The contents were stirred at room temperature for 16 hours. After completion of the reaction, the reaction mixture was acidified with 1.5N HCl and extracted with ethyl ethyl acetate (2×50 mL). Combined organic layers were dried over anhydrous Na₂SO₄, filtered and concentrated under vacuum to get 5-[2,6-difluoro-3-[(2,2,2-trifluoroacetyl)amino]benzoyl]-2-nitro-benzoic acid (5, 4 g, 3.93 mmol, 34% yield) as a brown gummy liquid. UPLC-MS (ES+): m/z 417.0 [M+H]⁺.

### Step 4

A 250 mL single-neck round bottom flask containing a well stirred solution of 5-[2,6-difluoro-3-[(2,2,2-trifluoroacetyl)amino]benzoyl]-2-nitro-benzoic acid (5, 4 g, 9.56 mmol) in EtOH (70 mL) was purged with nitrogen gas for 5 min and palladium, 10% on carbon (2 g, 18.79 mmol) was added under nitrogen atmosphere. The reaction mixture was stirred at room temperature under hydrogen atmosphere (bladder) for 16 hours. After completion of the reaction, the reaction mixture was filtered on a celite bed and the celite bed was washed with ethyl acetate (500 mL). Filtrate was concentrated under reduced pressure to afford 2-amino-5-[2,6-difluoro-3-[(2,2,2-trifluoroacetyl)amino]benzoyl]benzoic acid (6, 3.5 g, 4.07 mmol, 43% yield) as a brown solid. UPLC-MS (ES+): m/z 389.0 [M+H]⁺.

### Step 5

Into a 20 mL sealed-vial containing a well-stirred solution of 2-amino-5-[2,6-difluoro-3-[(2,2,2-trifluoroacetyl)amino]benzoyl]benzoic acid (6, 969.24 mg, 2.50 mmol) in 1,4-dioxane (5 mL) was added tert-butyl 2-amino-7-azaspiro[3.5]nonane-7-carboxylate (7, 500 mg, 2.08 mmol), triethylorthoformate (6.24 mmol, 1.04 mL) and AcOH (416.08 mol, 23.80 L) at 25° C. The reaction mixture was heated to 110° C. for 16 hours. After completion of the reaction, the reaction mixture was filtered through a celite bed, and the bed was washed with THF (10 mL). Filtrate was concentrated under reduced pressure to afford a crude residue which was purified by reverse phase by using RediSep Rf Gold® 100 g column and [Mobile phase A: 10 mM NH₄HCO₃ in mill-Q water; Mobile phase B: acetonitrile]. Fractions were collected and lyophilized to afford tert-butyl 2-[6-[2,6-difluoro-3-[(2,2,2-trifluoroacetyl)amino]benzoyl]-4-oxo-quinazolin-3-yl]-7-azaspiro[3.5]nonane-7-carboxylate (8, 200 mg, 294.73 mol, 14% yield) as an off-white solid. UPLC-MS (ES+): m/z 619.0 [M+H]⁺.

### Step 6

Into a 20 mL glass pressure vial containing a well-stirred solution tert-butyl 2-[6-[2,6-difluoro-3-[(2,2,2-trifluoroacetyl)amino]benzoyl]-4-oxo-quinazolin-3-yl]-7-azaspiro[3.5]nonane-7-carboxylate (8, 400 mg, 644.57 mol) in EtOH (5.04 mL) was added n-butylamine (3.51 mmol, 348.31 L) at room temperature. The reaction mixture was heated to 60° C. for 16 hours. After completion of the reaction, the reaction mixture was filtered, and the filtrate was evaporated under reduced pressure to give a crude residue which was purified by reverse phase by using RediSep Rf Gold® 100 g column and [Mobile phase A: 10 mM NH₄HCO₃ in milli-Q water; Mobile phase B: acetonitrile]. Fraction was lyophilized to afford tert-butyl 2-[6-(3-amino-2,6-difluoro-benzoyl)-4-oxo-quinazolin-3-yl]-7-azaspiro[3.5]nonane-7-carboxylate (9, 300 mg, 496.30 mol, 85% yield) as an off-white solid.

UPLC-MS (ES+): m/z 525.3 [M+H]⁺.

### Step 7

Into a 20 mL sealed-vial containing a well-stirred solution of tert-butyl 2-[6-(3-amino-2,6-difluoro-benzoyl)-4-oxo-quinazolin-3-yl]-7-azaspiro[3.5]nonane-7-carboxylate (9, 400 mg, 762.55 mol) and N-ethyl-N-methyl-sulfonyl chloride (10, 600.96 mg, 3.81 mmol, 470.12 L) in pyridine (4.81 mL) at ambient temperature under nitrogen atmosphere and the reaction mixture was stirred at 80° C. for 16 hours. Reaction mixture was concentrated under reduced pressure to give a crude residue which was purified by flash silica-gel (230-400 mesh; 20 g) column chromatography on using 0-100% ethyl acetate in hexane as an eluent to afford tert-butyl 2-[6-[3-[[ethyl(methyl)sulfamoyl]amino]-2,6-difluoro-benzoyl]-4-oxo-quinazolin-3-yl]-7-azaspiro[3.5]nonane-7-carboxylate (11, 250 mg, 355.96 mol, 47% yield) as a gummy liquid. UPLC-MS (ES+): m/z 646.1 [M+H]⁺.

### Step 8

Into a 50 mL single-neck round-bottom flask containing a well-stirred solution of tert-butyl 2-[6-[3-[[ethyl(methyl)sulfamoyl]amino]-2,6-difluoro-benzoyl]-4-oxo-quinazolin-3-yl]-7-azaspiro[3.5]nonane-7-carboxylate (11, 350 mg, 542.03 mol) in anhydrous dichloromethane (2.88 mL) was added trifluoroacetic acid (5.42 mmol, 414.78 L) at 0° C. under nitrogen atmosphere. The resulting suspension was stirred at room temperature for 3 hours. After completion of the reaction, the reaction mixture was concentrated under reduced pressure get a crude residue which was triturated with methyl tert-butyl ether (2×4 mL) to afford 3-(7-azaspiro[3.5]nonan-2-yl)-6-[3-[[ethyl(methyl)sulfamoyl]amino]-2,6-difluoro-benzoyl]-4-oxo-quinazoline (A-34, 370 mg, 419.74 mol, 77% yield) as an off-white solid. UPLC-MS (ES+): m/z 546.5 [M+H]⁺.

### Synthesis A35: Synthesis of (3R)-3-[6-[3-[[ethyl(methyl)sulfamoyl]amino]-2,6-difluoro-benzoyl]-4-oxo-quinazolin-3-yl]-1-oxa-8-azaspiro[4.5]decane (A-35)

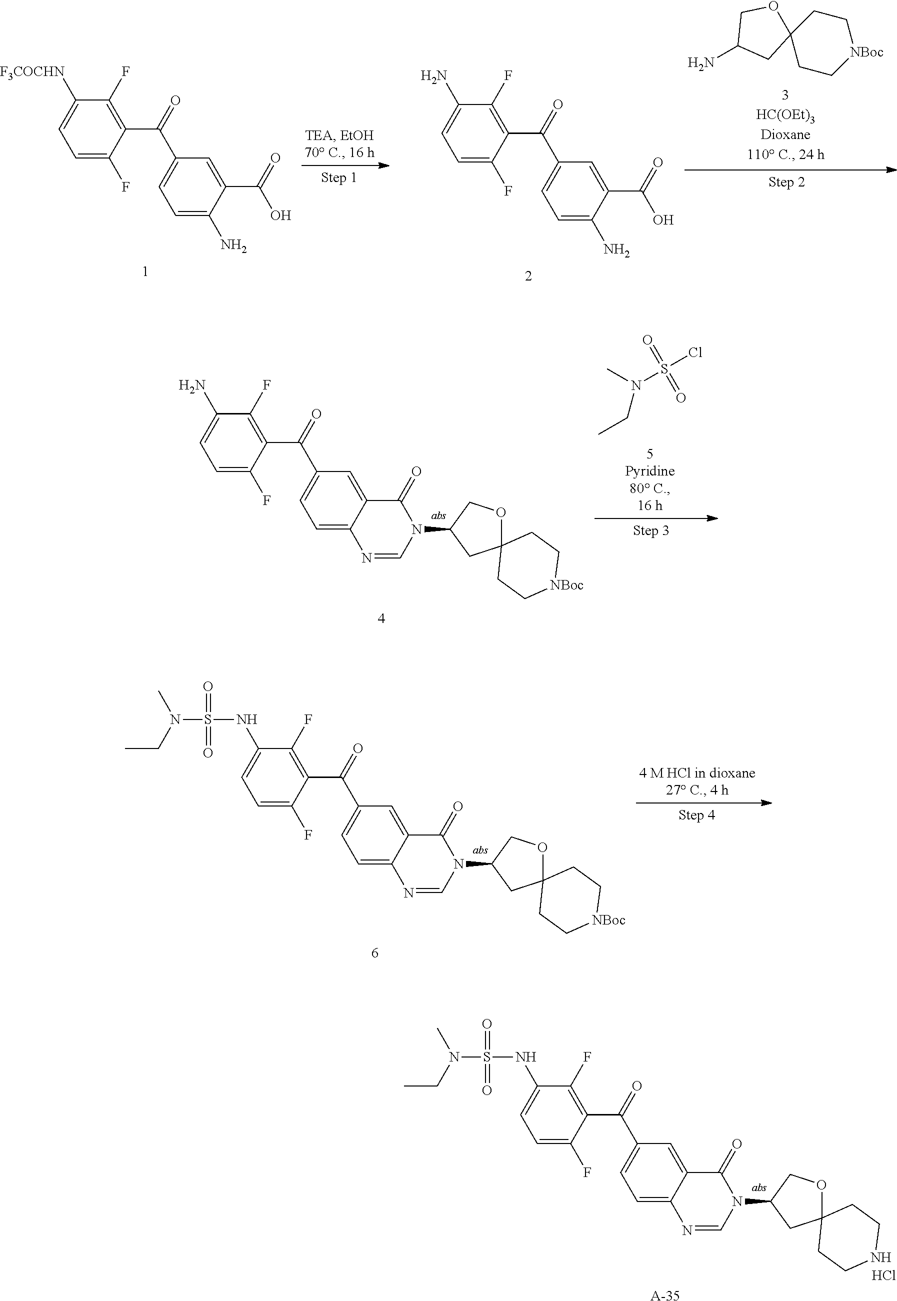

### Step 1

Into a 40 mL glass pressure vial containing a well-stirred solution 2-amino-5-[2,6-difluoro-3-[(2,2,2-trifluoroacetyl)amino]benzoyl]benzoic acid (1, 3 g, 7.73 mmol) in EtOH (8.70 mL) was added triethylamine (1.42 g, 14.04 mmol, 1.96 mL) at room temperature. The reaction mixture was stirred at 70° C. for 16 hours. After completion of the reaction, the reaction mixture was filtered and the filtrate was evaporated under reduced pressure to get crude, which was purified by reverse phase column chromatography (Redisep R_{f} Gold®reversed-phase C18-teledyne ISCO, 120 g), [Mobile phase A: NH₄HCO₃ in milli-Q water; Mobile phase B: acetonitrile]. The fractions were lyophilized to afford 2-amino-5-(3-amino-2,6-difluoro-benzoyl)benzoic acid (2, 1 g, 3.32 mmol, 47% yield) as an off-white solid. UPLC-MS (ES+): m/z 293.0 [M+H]⁺.

### Step 2

Into a 40 mL pressure vial containing a well-stirred solution of 2-amino-5-(3-amino-2,6-difluoro-benzoyl)benzoic acid (2, 1 g, 3.42 mmol) and tert-butyl 3-amino-1-oxa-8-azaspiro[4.5]decane-8-carboxylate (3, 1.14 g, 4.45 mmol) in 1,4-dioxane (5 mL) was added Triethylorthoformate (10.27 mmol, 1.71 mL) and catalytic amount of acetic acid (684.38 mol, 39.14 μL) at room temperature. The reaction mixture was stirred at 110° C. for 24 hours. After completion of the reaction, the reaction mixture was filtered through celite bed and bed was washed with THF (10 mL). Filtrate was concentrated under reduced pressure to afford a crude residue, which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fractions were lyophilized to afford tert-butyl 3-[6-(3-amino-2,6-difluoro-benzoyl)-4-oxo-quinazolin-3-yl]-1-oxa-8-azaspiro[4.5]decane-8-carboxylate (4, 380 mg, 684.91 mol, 20% yield) as an off-white solid.

UPLC-MS (ES+): m/z 541.30 [M+H]+

### Step 3

Into a 20 mL pressure vial containing a well-stirred solution of tert-butyl(3R)-3-[6-(3-amino-2,6-difluoro-benzoyl)-4-oxo-quinazolin-3-yl]-1-oxa-8-azaspiro[4.5]decane-8-carboxylate (4, 210 mg, 388.49 mol) in pyridine (2 mL) was added N-ethyl-N-methyl-sulfamoyl chloride (5, 1.17 mmol, 143.71 L) at ambient temperature under nitrogen atmosphere and the reaction mixture was stirred at 80° C. for 16 hours. After completion of the reaction, the reaction mixture was concentrated under reduced pressure to get a crude residue which was purified by flash silica-gel (230-400 mesh; 20 g) column chromatography using 0-100% ethyl acetate in hexane as an eluent to afford tert-butyl(3R)-3-[6-[3-[[ethyl(methyl)sulfamoyl]amino]-2,6-difluoro-benzoyl]-4-oxo-quinazolin-3-yl]-1-oxa-8-azaspiro[4.5]decane-8-carboxylate (6, 160 mg, 224.73 mol, 58% yield) as a gummy liquid. UPLC-MS (ES+): m/z 660.2 [M+H]⁺.

### Step 4

Into a 50 mL single-neck round-bottom flask containing a well-stirred solution of tert-butyl(3R)-3-[6-[3-[[ethyl(methyl)sulfamoyl]amino]-2,6-difluoro-benzoyl]-4-oxo-quinazolin-3-yl]-1-oxa-8-azaspiro[4.5]decane-8-carboxylate (6, 170 mg, 256.91 mol) in anhydrous dichloromethane (3 mL) was added trifluoroacetic acid (2.57 mmol, 196.60 μL) at 0° C. under nitrogen atmosphere. The resulting suspension was stirred at room temperature for 4 hours. After completion of the reaction, the reaction mixture was concentrated under reduced pressure to get a crude residue which was triturated with methyl tert-butyl ether (2×4 mL) to afford (3R)-3-[6-[3-[[ethyl(methyl)sulfamoyl]amino]-2,6-difluoro-benzoyl]-4-oxo-quinazolin-3-yl]-1-oxa-8-azaspiro[4.5]decane (A-35, 160 mg, 216.76 mol, 84% yield, TFA salt) as an off-white solid. UPLC-MS (ES+): m/z 562.0 [M+H]⁺.

### Synthesis A36: Synthesis of 6-[2-cyano-3-[[ethyl(methyl)sulfamoyl]amino]-6-fluoro-phenoxy]-4-oxo-3-(2-oxospiro[3.3]heptan-6-yl)quinazoline (A-36)

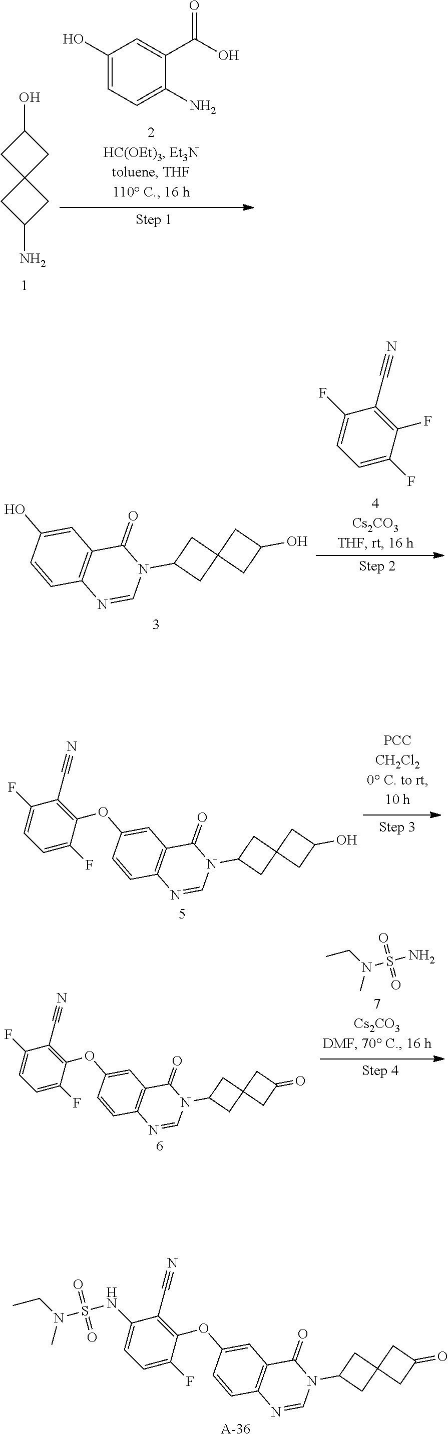

### Step 1

Into a 100 mL sealed tube containing a well stirred solution of 6-aminospiro[3.3]heptan-2-ol (1, 500 mg, 3.06 mmol, HCl salt) in THF (2 mL) & Toluene (6.5 mL) was added triethylamine (3.06 mmol, 425.86 μL) and stirred for 10 min. After that, 2-amino-5-hydroxy-benzoic acid (2, 467.89 mg, 3.06 mmol) was added followed by triethylorthoformate (12.22 mmol, 2.03 mL) to the reaction mixture at room temperature under nitrogen atmosphere. The reaction mixture was stirred at 110° C. for 16 hours. After completion of the reaction, the reaction mixture was quenched with water (30 mL) and extracted with ethyl acetate (3×50 mL). The combined organic layer were washed with saturated NaHCO₃ solution (50 mL) and dried over anhydrous Na₂SO₄, filtered and concentrated under reduced pressure to get a crude residue, which was purified by flash silica-gel (200-400 mesh) column chromatography using a gradient of 0-10% methanol/dichloromethane to afford 6-hydroxy-3-(2-hydroxyspiro[3.3]heptan-6-yl)quinazolin-4-one (3, 354 mg, 1.23 mmol, 40% yield) as a pale yellow solid. UPLC-MS (ES+): m/z 273.1 [M+H]⁺.

### Step 2

Into a 40 mL pressure vial containing a well stirred solution of 6-hydroxy-3-(2-hydroxyspiro[3.3]heptan-6-yl)quinazolin-4-one (3, 472 mg, 1.73 mmol) in THF (10 mL) were added Cs₂CO₃ (1.13 g, 3.47 mmol) and 2,3,6-trifluorobenzonitrile (4, 2.08 mmol, 240.27 μL) under nitrogen atmosphere. The reaction mixture was stirred at room temperature for 16 hours. After completion of the reaction, the reaction mixture was diluted with water (20 mL), extracted with ethyl acetate (3×50 mL). The organic layer were washed with saturated NaCl solution (20 mL) and dried over anhydrous Na₂SO₄ and concentrated under reduced pressure to get a crude residue, which was purified by flash silica-gel (200-400 mesh) column chromatography using a gradient of 0-10% methanol/dichloromethane to afford 3,6-difluoro-2-[3-(2-hydroxyspiro[3.3]heptan-6-yl)-4-oxo-quinazolin-6-yl]oxy-benzonitrile (5, 518 mg, 1.11 mmol, 64% yield) as a white solid. UPLC-MS (ES+): m/z 410.1 [M+H]⁺.

### Step 3

Into a 50 mL single neck round-bottom flask containing a well stirred solution of 3,6-difluoro-2-[3-(2-hydroxyspiro[3.3]heptan-6-yl)-4-oxo-quinazolin-6-yl]oxy-benzonitrile (5, 510 mg, 1.25 mmol) in dichloromethane (23 mL) was added pyridinium chlorochromate (671.32 mg, 3.11 mmol) at 0° C. under nitrogen atmosphere followed by addition of celite (150 mg, 1.25 mmol). The reaction mixture was stirred at room temperature for 10 hours. After completion of the reaction, the reaction mixture was filtered through celite, and the filtrate was concentrated under reduced pressure to get a crude residue, which was purified by flash silica-gel (230-400 mesh) column chromatography using a gradient of 0-100% ethyl acetate/Hexane to afford 3,6-difluoro-2-[4-oxo-3-(2-oxospiro[3.3]heptan-6-yl)quinazolin-6-yl]oxy-benzonitrile (6, 280 mg, 670.43 mol, 54% yield) as an off-white solid. UPLC-MS (ES+): m/z 408.0 [M+H]⁺.

### Step 4

Into a 10 mL pressure vial containing a well stirred solution of 3,6-difluoro-2-[4-oxo-3-(2-oxospiro[3.3]heptan-6-yl)quinazolin-6-yl]oxy-benzonitrile (6, 280 mg, 687.34 mol) in N,N-dimethylformamide (4 mL) were added Cs₂CO₃ (671.84 mg, 2.06 mmol) and [methyl(sulfamoyl)amino]ethane (7, 237.46 mg, 1.72 mmol) at room temperature under nitrogen atmosphere. The reaction mixture was stirred at 70° C. for 16 hours. After completion of the reaction, the reaction mixture was diluted with water (20 mL) and extracted with ethyl acetate (3×30 mL), the combined organic layer were washed with saturated NaCl solution (20 mL) and dried over anhydrous Na₂SO₄ and concentrated under reduced pressure to afford crude 6-[2-cyano-3-[[ethyl(methyl)sulfamoyl]amino]-6-fluoro-phenoxy]-4-oxo-3-(2-oxospiro[3.3]heptan-6-yl)quinazoline (A-36, 190 mg, 193.45 mol, 28% yield) as pale yellow liquid. UPLC-MS (ES+): m/z 526.0 [M+H]⁺.

### Synthesis A37: Synthesis of 6-[2-cyano-3-[[ethyl(methyl)sulfamoyl]amino]-6-fluoro-phenoxy]-4-oxo-3-(7-oxospiro[3.5]nonan-2-yl)quinazoline (A-37)

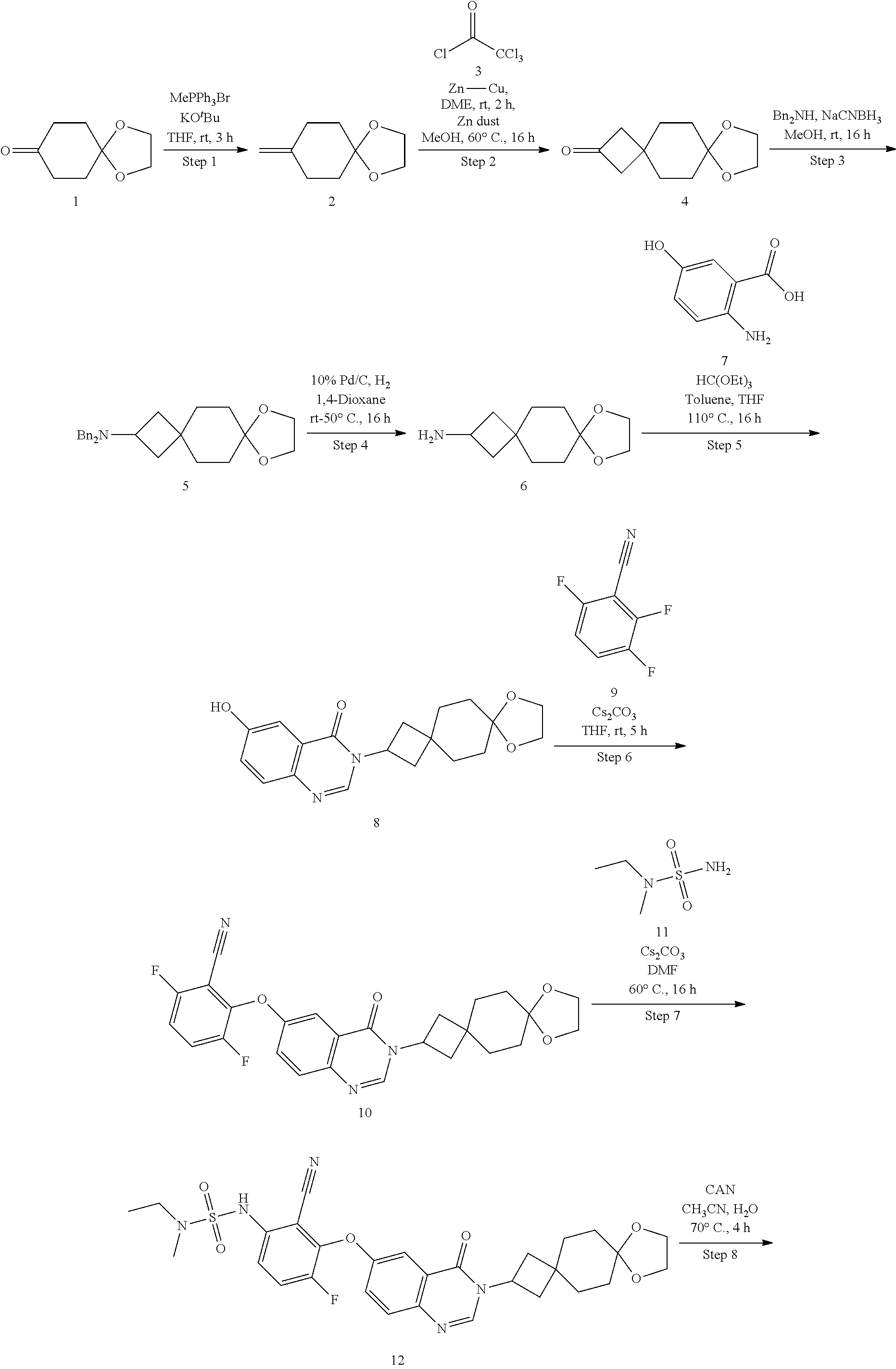

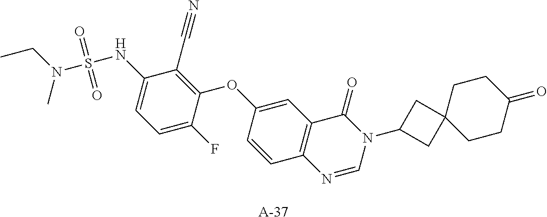 [SMILES: CCN(C)S(=O)(=O)Nc1ccc(F)c(Oc2ccc3ncn(C4CC5(CCC(=O)CC5)C4)c(=O)c3c2)c1C#N ; "A-37"]

### Step 1

Into a 250 mL double-neck round-bottom flask containing a suspension of Potassium tert-butoxide (8.98 g, 80.04 mmol) in THF (40 mL) was added methyl triphenylphosphoniumbromide (28.59 g, 80.04 mmol) portion wise at 0° C. After stirring for 1 h, 1,4-dioxaspiro[4.5]decan-8-one (1, 5 g, 32.01 mmol) in THF (20 mL) was added. The resulting mixture was allowed to room temperature and stirred for 3 hours. After completion of the reaction, the reaction mixture was poured into saturated aq. NH₄C₁ (50 mL) and extracted with ethyl acetate (2×100 mL). The combined organic layer was washed with water (100 mL), saturated NaCl (100 mL) and dried over anhydrous Na₂SO₄, concentrated under reduced pressure to get a crude product which was purified by flash chromatography (silica gel, 0-30% ethyl acetate in Hexane) to afford 8-methylene-1,4-dioxaspiro[4.5]decane (2, 3.9 g, 24.03 mmol, 75% yield) as a colorless thick liquid. ¹H NMR (400 MHz, DMSO-d₆): δ 4.67 (s, 2H), 3.88 (s, 4H), 2.22-2.19 (m, 4H), 1.62-1.59 (m, 4H).

### Step 2

Into a 250 mL double-neck round-bottom flask containing a suspension of 8-methylene-1,4-dioxaspiro[4.5]decane (2, 2.6 g, 16.86 mmol) and zinc-copper (8.70 g, 67.44 mmol) in 1,2-DME (26 mL) was added 2,2,2-trichloroacetyl chloride (3, 33.72 mmol, 3.78 mL) drop wise at room temperature. After stirring at room temperature for 1 h, methanol (9 mL) was added, the mixture was cooled to −5° C. and Zinc powder (11.03 g, 168.61 mmol) was added in portion over the period of 1 h at −5° C. Reaction mixture was allowed to room temperature and stirred for 2 hours. After completion of the reaction, celite was added. The resulting thick mass was filtered over celite pad, and the cake was washed with excess ethyl acetate (100 mL). The organic phase was washed with saturated NaCl (100 mL) and dried over Na₂SO₄ and concentrated under reduced pressure to get crude. Crude product was dissolved in methanol (15 mL) followed by addition of zinc powder (11.03 g, 168.61 mmol) and reaction mixture was stirred at 60° C. for 16 hours. The resulting thick mass was filtered over celite pad and washed with ethyl acetate (100 mL), filtrate was concentrated under reduced pressure to get crude which was purified by flash column chromatography (silica gel, 230-400 mesh) using 0-100% ethyl acetate/hexane as gradient to afford 8,11-dioxadispiro[3.2.4⁷.2⁴]tridecan-2-one (4, 1.3 g, 6.62 mmol, 39% yield) as a gummy liquid. ¹H NMR (400 MHz, DMSO-d₆): δ 3.87 (s, 4H), 2.78 (s, 4H), 1.73-1.69 (m, 4H), 1.57-1.54 (m, 4H)

### Step 3

Into a 50 mL double-neck round-bottom flask containing a suspension of 8,11-dioxadispiro[3.2.4⁷.2⁴]tridecan-2-one (4, 1.3 g, 6.62 mmol) in methanol (20 mL) was added dibenzyl amine (9.94 mmol, 1.91 mL) and stirred at room temperature for 2 hours. Subsequently, Sodium cyanoborohydride (1.67 g, 26.50 mmol) was added and stirred at room temperature for 16 hours. After completion of the reaction, the reaction mixture was poured into water (50 mL) and extracted with ethyl acetate (2×40 mL). The combined organic layer was washed with water (30 mL), saturated NaCl (30 mL) and dried over anhydrous Na₂SO₄, concentrated under reduced pressure to get crude which was purified by flash chromatography (silica gel 230-400 mesh) using 0-100% ethyl acetate in hexane to afford N,N-dibenzyl-8,11-dioxadispiro[3.2.4⁷.2⁴]tridecan-2-amine (5, 900 mg, 2.03 mmol, 31% yield) as a colorless thick liquid. UPLC-MS (ES+): m/z 378.2 [M+H]⁺.

### Step 4

Into a 25 mL Tiny clave glass flask containing a well stirred solution of N,N-dibenzyl-8,11-dioxadispiro[3.2.4⁷.2⁴]tridecan-2-amine (5, 900 mg, 2.38 mmol) in 1,4-dioxane (15 mL) was added 10% Pd/C (1.2 g, 2.38 mmol) at room temperature under nitrogen atmosphere. The resulting suspension was stirred at 50° C. under hydrogen atmosphere (70 Psi) for 16 hours. After completion of the reaction, the reaction mixture was filtered through celite bed and celite bed was washed with ethyl acetate/dichloromethane (1:1, 300 mL), evaporate the filtrate under reduced pressure to afford 8,11-dioxadispiro[3.2.4⁷.2⁴]tridecan-2-amine (6, 450 mg, 2.03 mmol, 85% yield) as a gummy liquid. UPLC-MS (ES+): m/z 198.2 [M+H]⁺.

### Step 5

Into a 40 mL pressure glass vial containing a well stirred solution of 2-amino-5-hydroxy-benzoic acid (7, 350 mg, 2.29 mmol) in anhydrous THF (2 mL), toluene (8 mL) were added 8,11-dioxadispiro[3.2.4⁷.2⁴]tridecan-2-amine (6, 450.88 mg, 2.29 mmol) and triethyl orthoformate (4.57 mmol, 760.32 L) at room temperature under nitrogen atmosphere. After complete addition, the reaction mixture was stirred at 110° C. for 16 h under nitrogen atmosphere. After completion of the reaction, the reaction mixture was diluted with water (50 mL) and extracted with ethyl acetate (2×40 mL). The combined organic layer was dried over anhydrous Na₂SO₄, filtered and concentrated under reduced pressure to get crude 3-(8,11-dioxadispiro[3.2.4⁷.2⁴]tridecan-2-yl)-6-hydroxy-quinazolin-4-one (8, 450 mg, 933.15 mol, 41% yield) as a brown color solid. UPLC-MS (ES+): m/z 343.3 [M+H]⁺.

### Step 6

Into a 50 mL single-neck round-bottom flask containing a well stirred solution of 3-(8,11-dioxadispiro[3.2.4⁷.2⁴]tridecan-2-yl)-6-hydroxy-quinazolin-4-one (8, 450 mg, 1.31 mmol) in THF (10 mL) was added Cs₂CO₃ (1.28 g, 3.94 mmol) followed by 2,3,6-trifluorobenzonitrile (9, 1.58 mmol, 182.18 μL) at 0° C. Resulting reaction mixture was stirred at room temperature for 5 hours. After completion, the reaction mixture was diluted with water (50 mL) and extracted with ethyl acetate (2×50 mL), combined organic layer was dried over anhydrous Na₂SO₄, filtered and concentrated under reduced pressure to get crude which was purified by flash column chromatography (100-200 mesh) using 0-100% ethyl acetate/hexane as an eluent and compound was eluted at 30-35% of mobile phase to afford 2-[3-(8,11-dioxadispiro[3.2.4⁷.2⁴]tridecan-2-yl)-4-oxo-quinazolin-6-yl]oxy-3,6-difluoro-benzonitrile (10, 400 mg, 825.90 mol, 63% yield) as an off-white solid. UPLC-MS (ES+): m/z 480.4 [M+H]⁺.

### Step 7

Into a 20 mL glass vial with pressure released cap containing a well stirred solution of 2-[3-(8,11-dioxadispiro[3.2.4⁷.2⁴]tridecan-2-yl)-4-oxo-quinazolin-6-yl]oxy-3,6-difluoro-benzonitrile (10, 400 mg, 834.25 mol) in N,N-dimethylformamide (5 mL) was added Cs₂CO₃ (815.44 mg, 2.50 mmol) followed by [methyl(sulfamoyl)amino]ethane (11, 172.92 mg, 1.25 mmol) at room temperature. Resulting reaction mixture was stirred at 60° C. for 16 hours. After completion of the reaction, the reaction mixture was diluted with water (10 mL) and extracted with ethyl acetate (2×10 mL). Combined organic layer was dried over anhydrous Na₂SO₄, filtered and concentrated under reduced pressure to get a crude which was purified by flash silica-gel (230-400 mesh) column with 0-100% ethyl acetate/hexane to afford 6-[2-cyano-3-[[ethyl(methyl)sulfamoyl]amino]-6-fluoro-phenoxy]-3-(8,11-dioxadispiro[3.2.4⁷.2⁴]tridecan-2-yl)-4-oxo-quinazoline (12, 405 mg, 555.67 mol, 67% yield) as an off-white solid. UPLC-MS (ES+): m/z 598.1 [M+H]⁺.

### Step 8

Into a 40 mL glass vial with pressure released cap containing a well stirred solution of 6-[2-cyano-3-[[ethyl(methyl)sulfamoyl]amino]-6-fluoro-phenoxy]-3-(8,11-dioxadispiro[3.2.4⁷.2⁴]tridecan-2-yl)-4-oxo-quinazoline (12, 400 mg, 669.28 mol) in acetonitrile (8 mL), Water (2 mL) was added Cerium(IV) ammonium nitrate (156.67 mg, 334.64 mol) at room temperature. The resulting mixture was stirred at 70° C. for 4 hours. After completion, the reaction mixture was diluted with water (30 mL) and extracted with ethyl acetate (2×30 mL), combined organic layer was washed with saturated NaCl (50 mL), dried over anhydrous Na₂SO₄ and concentrated under reduced pressure to get 6-[2-cyano-3-[[ethyl(methyl)sulfamoyl]amino]-6-fluoro-phenoxy]-4-oxo-3-(7-oxospiro[3.5]nonan-2-yl)quinazoline (A-37, 305 mg, 451.77 mol, 68% yield) as a brown color solid. UPLC-MS (ES+): m/z 554.2 [M+H]⁺.

### Synthesis A38: Synthesis of 6-[2-cyano-3-[[ethyl(methyl)sulfamoyljaminoj-6-fluoro-phenoxy]-4-oxo-3-(4-oxocyclohexyl)quinazoline (A-38)

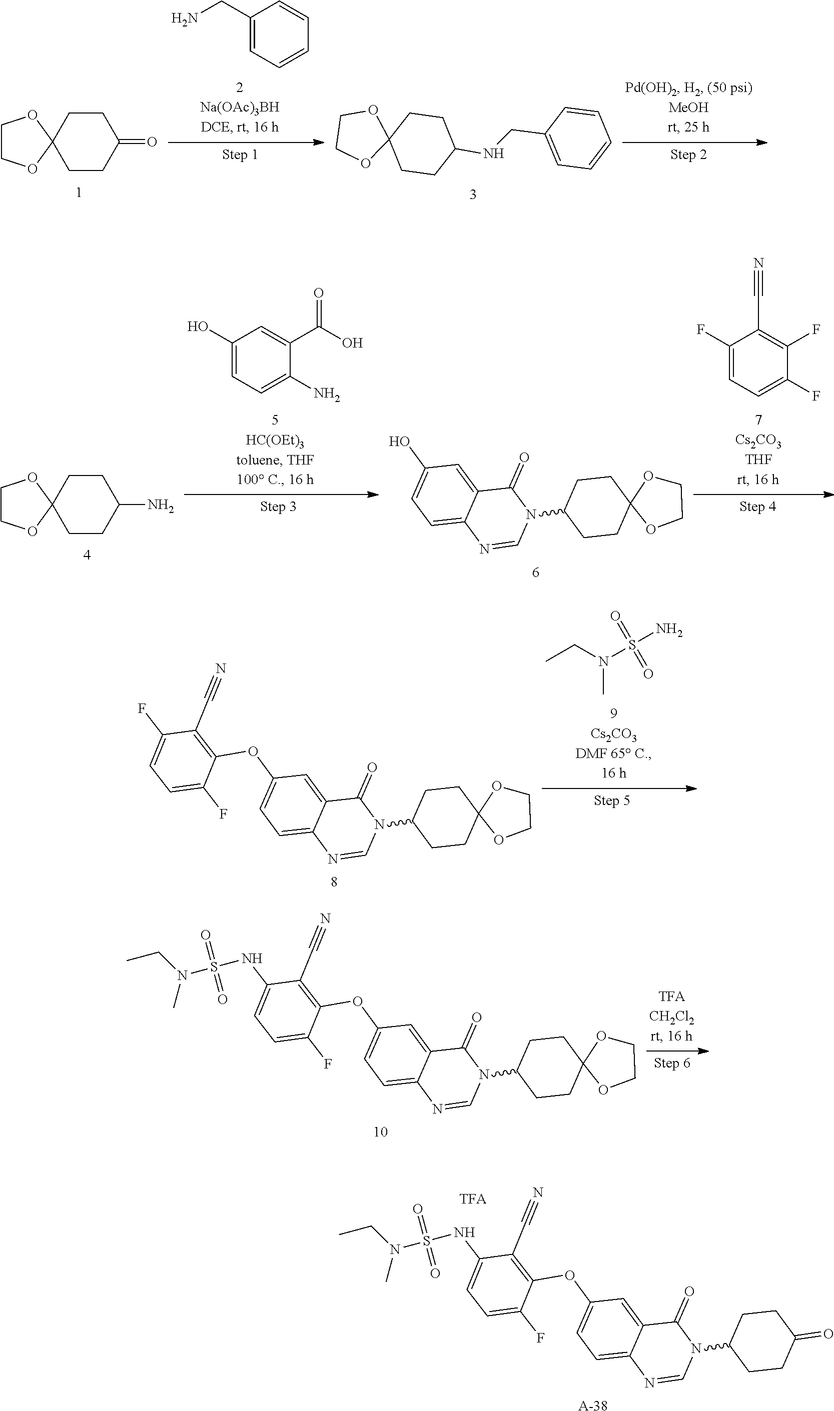

### Step 1

Into a 50 mL double-neck round bottom flask containing a well stirred solution of 1,4-dioxaspiro[4.5]decan-8-one (1, 5.0 g, 32.01 mmol) and phenylmethanamine (2,160.07 mmol, 17.48 mL) in 1,2-dichloroethane (150 mL) were added sodium triacetoxy borohydride (20.36 g, 96.04 mmol) and acetic acid (64.03 mmol, 3.66 mL) at 0° C. under nitrogen atmosphere. The resulting mixture was stirred at room temperature for 16 hours. After completion of the reaction, the reaction mixture was diluted with water (150 mL) and extracted with ethyl acetate (3×100 mL). The combined organic layer was washed with saturated NaCl (100 mL), dried over anhydrous Na₂SO₄ and concentrated under reduced pressure to get a crude residue, which was purified by column chromatography (250 g, 100-200 silica gel mesh) using a gradient of 0-100% ethyl acetate in hexane to afford N-benzyl-1,4-dioxaspiro[4.5]decan-8-amine (3, 5.5 g, 20.90 mmol, 65% yield) as a yellow liquid. UPLC-MS (ES+): m/z 248.4 [M+H]⁺.

### Step 2

Into a 250 mL single-neck round bottom flask containing a well-stirred solution of N-benzyl-1,4-dioxaspiro[4.5]decan-8-amine (3, 5.5 g, 22.24 mmol) in methanol (100 mL) was added Pd(OH)2 (50% wet) (1.87 g, 13.34 mmol) at ambient temperature under nitrogen atmosphere. The reaction mixture was stirred at room temperature under hydrogen atmosphere (bladder pressure) for 25 hours. After completion of the reaction, the reaction mixture was filtered through a pad of celite and washed with methanol (100 mL). The filtrate was concentrated under reduced pressure to afford 1,4-dioxaspiro[4.5]decan-8-amine (4, 3.5 g, 22.04 mmol, 99% yield) as a brown gum. UPLC-MS (ES+): m/z 158.2 [M+H]⁺.

### Step 3

Into a 50 mL two-neck round-bottom flask containing a well stirred solution of 1,4-dioxaspiro[4.5]decan-8-amine (4, 3.5 g, 22.26 mmol) and 2-amino-5-hydroxy-benzoic acid (5, 3.75 g, 24.49 mmol) in toluene (50 mL) and THF (10 mL) was added triethyl orthoformate (89.05 mmol, 14.81 mL) at room temperature under nitrogen atmosphere and stirred at 100° C. for 16 hours. After completion of the reaction, the reaction mixture was allowed to room temperature and poured into cold water (100 mL). The layers were separated, and the aqueous layer was extracted with ethyl acetate (2×100 mL). The combined organic layer was washed with saturated NaHCO₃ solution (100 mL), saturated NaCl (100 mL), dried over anhydrous Na₂SO₄, filtered and concentrated under reduced pressure to get a crude residue, which was purified by flash column chromatography (100 g, 230-400 silica-gel mesh) with 0-100% ethyl acetate in hexane, while desired product was eluting at 20-30% of the mobile phase to afford 3-(1,4-dioxaspiro[4.5]decan-8-yl)-6-hydroxy-quinazolin-4-one (6, 1.5 g, 4.54 mmol, 20% yield) as an off-white solid. UPLC-MS (ES+): m/z 303.0 [M+H]⁺.

### Step 4

Into a 25 mL single-neck round-bottom flask containing a well stirred solution of 3-(1,4-dioxaspiro[4.5]decan-8-yl)-6-hydroxy-quinazolin-4-one (6, 1.5 g, 451.50 mmol) and 2,3,6-trifluorobenzonitrile (7,17.82 mmol, 2.06 mL) in THF (30 mL) was added Cs₂CO₃ (2.9 g, 8.90 mmol) at room temperature under nitrogen atmosphere. The reaction mixture was stirred at room temperature for 16 hours. After completion of the reaction, the reaction mixture was diluted with water (100 mL) and extracted with ethyl acetate (3×50 mL). The combined organic layer was washed with saturated NaCl (50 mL), dried over anhydrous Na₂SO₄ and concentrated under reduced pressure to get a crude residue, which was purified by column chromatography (100 g, 100-200 silicagel mesh) using a gradient of 0-100% ethyl acetate in hexane to afford 2-[3-(1,4-dioxaspiro[4.5]decan-8-yl)-4-oxo-quinazolin-6-yl]oxy-3,6-difluoro-benzonitrile (8, 1.5 g, 3.31 mmol, 7.33e-1% yield) as an off-white solid. UPLC-MS (ES+): m/z 440.0 [M+H]⁺.

### Step 5

Into a 30 mL pressure glass vial containing a well stirred solution of 2-[3-(1,4-dioxaspiro[4.5]decan-8-yl)-4-oxo-quinazolin-6-yl]oxy-3,6-difluoro-benzonitrile (8, 1.5 g, 3.41 mmol) and [methyl(sulfamoyl)amino]ethane (9, 943.46 mg, 6.83 mmol) in N,N-dimethylformamide (15 mL) was added Cs₂CO₃ (3.34 g, 10.24 mmol) at room temperature under nitrogen atmosphere. The resulting mixture was stirred at 65° C. for 16 hours. After completion of the reaction, the reaction mixture was diluted with water (100 mL) and extracted with ethyl acetate (3×50 mL). The combined organic layer was washed with saturated NaCl (100 mL), dried over anhydrous Na₂SO₄, filtered and concentrated under reduced pressure to afford 6-[2-cyano-3-[[ethyl(methyl)sulfamoyl]amino]-6-fluoro-phenoxy]-3-(1,4-dioxaspiro[4.5]decan-8-yl)-4-oxo-quinazoline (10, 1.3 g, 1.51 mmol, 44% yield) as an off-white solid. UPLC-MS (ES+): m/z 558.4 [M+H]⁺.

### Step 6

Into a 100 mL single neck round-bottom flask containing a well stirred solution of 6-[2-cyano-3-[[ethyl(methyl)sulfamoyl]amino]-6-fluoro-phenoxy]-3-(1,4-dioxaspiro[4.5]decan-8-yl)-4-oxo-quinazoline (10, 1.3 g, 2.33 mmol) in dichloromethane (30 mL) was added trifluoroacetic acid (39.20 mmol, 3.00 mL) at 0° C. under nitrogen atmosphere and the reaction mixture was stirred at room temperature for 16 hours. After completion of the reaction, the reaction mixture was concentrated under reduced pressure to get a crude, which was triturated with methyl tert-butyl ether (2×5 mL) and dried under reduced pressure to afford 6-[2-cyano-3-[[ethyl(methyl)sulfamoyl]amino]-6-fluoro-phenoxy]-4-oxo-3-(4-oxocyclohexyl)quinazoline (A-38, 2.0 g, 1.60 mmol, 68% yield, TFA salt) as a brown gum. UPLC-MS (ES+): m/z 514.2 [M+H]⁺.

### Synthesis A39: Synthesis of 6-[2-cyano-3-[[ethyl(methyl)sulfamoyl]amino]-6-fluoro-phenoxy]-4-oxo-3-(4-piperidylmethyl)quinazoline (A-39)

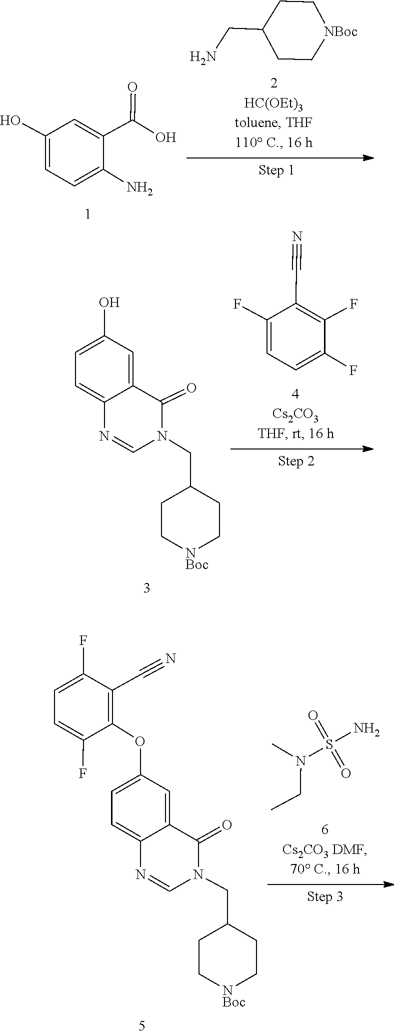

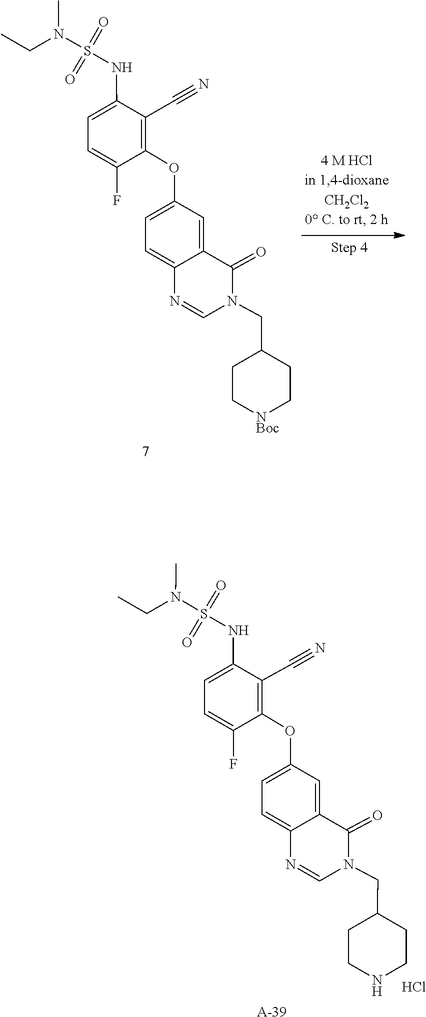 [SMILES: CCN(C)S(=O)(=O)Nc1ccc(F)c(Oc2ccc3ncn(CC4CCN(C(=O)OC(C)(C)C)CC4)c(=O)c3c2)c1C#N ; CCN(C)S(=O)(=O)Nc1ccc(F)c(Oc2ccc3ncn(CC4CCNCC4)c(=O)c3c2)c1C#N ; Cl ; "A-39"]

### Step 1

Into a 250 mL two neck round-bottom flask containing well stirred solution of 2-amino-5-hydroxy-benzoic acid (1, 3 g, 19.59 mmol) and tert-butyl 4-(aminomethyl)piperidine-1-carboxylate (2, 4.20 g, 19.59 mmol) in toluene (55 mL) and anhydrous THF (15 mL) was added diethoxymethoxyethane (29.39 mmol, 4.89 mL) at room temperature under nitrogen atmosphere. The resulting reaction mixture was stirred at 110° C. for 16 hours. After completion of the reaction, the reaction mixture was quenched with water (100 mL) and extracted with ethyl acetate (2×250 mL). Combined organic layer was washed with H₂O (150 mL), saturated saturated NaCl (150 mL), dried over anhydrous Na₂SO₄, filtered and concentrated under reduced pressure to get a crude residue which was triturated with 5% ethyl acetate in hexane to afford tert-butyl 4-[(6-hydroxy-4-oxo-quinazolin-3-yl)methyl]piperidine-1-carboxylate (3, 6.2 g, 16.22 mmol, 83% yield) as a brown solid. UPLC-MS (ES+): m/z 360.3 [M+H]⁺.

### Step 2

Into a 250 mL two neck round-bottom flask containing a well stirred solution of tert-butyl 4-[(6-hydroxy-4-oxo-quinazolin-3-yl)methyl]piperidine-1-carboxylate (3, 6.2 g, 16.22 mmol) in anhydrous THF (50.07 mL) were added Cs₂CO₃ (15.85 g, 48.65 mmol) and 2,3,6-Trifluorobenzonitrile (4, 19.46 mmol, 2.25 mL) at room temperature and resulting reaction mixture was stirred at the same temperature for 16 hours. After completion of the reaction, the reaction mixture was diluted with water (400 mL) and extracted with ethyl acetate (2×200 mL). Combined organic layer was washed with saturated NaCl solution (200 mL), dried over anhydrous Na₂SO₄, filtered and concentrated under reduced pressure to afford a crude residue which was purified by flash (100-200 mesh, 340 g) column chromatography using 0-100% ethyl acetate/hexane as an eluent while desired product was eluting at 50-60% of mobile phase to afford tert-butyl 4-[[6-(2-cyano-3,6-difluoro-phenoxy)-4-oxo-quinazolin-3-yl]methyl]piperidine-1-carboxylate (5, 4.1 g, 8.17 mmol, 50% yield) as an off-white solid. UPLC-MS (ES+): m/z 441.2 [M+H-^{t}Bu]⁺.

### Step 3

Into a 50 mL single neck round-bottom flask containing a well-stirred of tert-butyl 4-[[6-(2-cyano-3,6-difluoro-phenoxy)-4-oxo-quinazolin-3-yl]methyl]piperidine-1-carboxylate (5, 4.1 g, 8.18 mmol) in anhydrous N,N-dimethylformamide (20 mL) were added Cs₂CO₃ (7.99 g, 24.53 mmol) and [methyl(sulfamoyl)amino]ethane (6, 2.28 g, 16.35 mmol) at room temperature under nitrogen atmosphere. Resulting reaction mixture was stirred at 70° C. for 16 hours. After completion of the reaction, the reaction mixture was diluted with water (200 mL), filtered through filter paper to remove the fluorescent impurity and filtrate was extracted with ethyl acetate (2×200 mL). Combined organic layer was washed with cold water (150 mL), dried over anhydrous sodium sulphate, filtered and concentrated under reduced pressure to afford crude tert-butyl 4-[[6-[2-cyano-3-[[ethyl(methyl)sulfamoyl]amino]-6-fluoro-phenoxy]-4-oxo-quinazolin-3-yl]methyl]piperidine-1-carboxylate (7, 1.8 g, 2.50 mmol, 31% yield) as a brown gummy compound. UP-LC-MS(ES+): m/z 515.2 [M+H-Boc]⁺.

### Step 4

Into a 50 mL single neck round-bottom flask containing a well-stirred solution of tert-butyl 4-[[6-[2-cyano-3-[[ethyl(methyl)sulfamoyl]amino]-6-fluoro-phenoxy]-4-oxo-quinazolin-3-yl]methyl]piperidine-1-carboxylate (7, 1.8 g, 2.50 mmol) in anhydrous 1,4-dioxane (12.02 mL) was added 4M HCl in 1,4-dioxane (4 M, 11.70 mL) under nitrogen atmosphere at room temperature and resulting reaction mixture was stirred at the same temperature for 2 hours. After completion of the reaction, the reaction mixture was concentrated under reduced pressure to get a crude residue which was triturated with methyl tert-butyl ether to get 6-[2-cyano-3-[[ethyl(methyl)sulfamoyl]amino]-6-fluoro-phenoxy]-4-oxo-3-(4-piperidylmethyl)quinazoline (A-39, 1.3 g, 2.08 mmol, 83% yield, HCl Salt) as a brown gummy liquid. UPLC-MS (ES+): m/z 515.2 [M+H]⁺.

### Synthesis A40: Synthesis of 6-[2-cyano-3-[[ethyl(methyl)sulfamoyl]amino]-6-fluoro-phenoxy]-3-[(4-fluoro-4-piperidyl)methyl]-4-oxo-quinazoline (A-40)

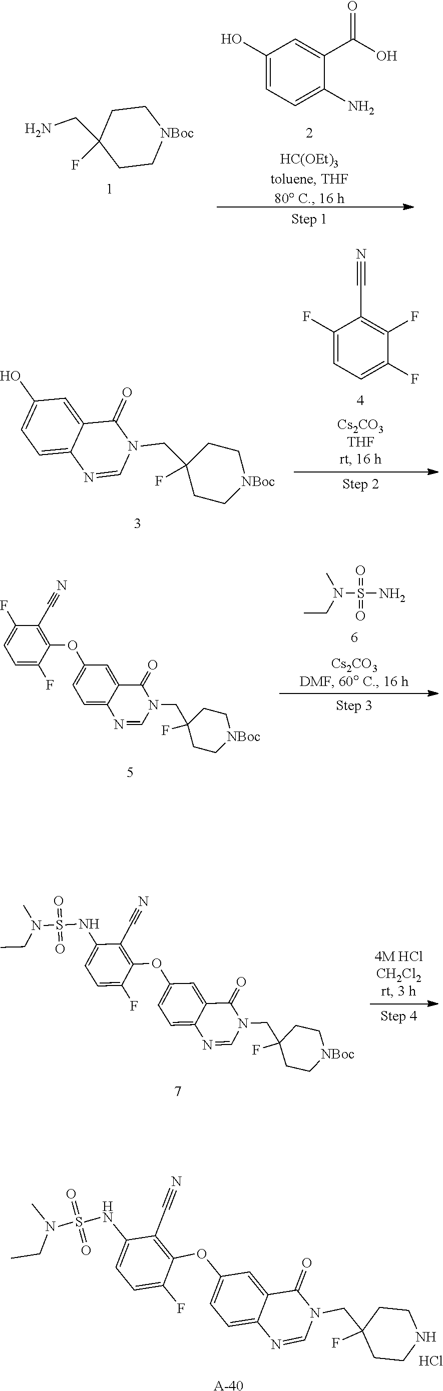

### Step 1

Into a 250 mL single-neck round-bottom flask containing a well stirred solution of tert-butyl 4-(aminomethyl)-4-fluoro-piperidine-1-carboxylate (1, 3.0 g, 12.91 mmol) and 2-amino-5-hydroxy-benzoic acid (2, 1.98 g, 12.91 mmol) in anhydrous toluene (60 mL) and anhydrous THF (30 mL) was added triethyl orthoformate (51.66 mmol, 8.59 mL) at room temperature under nitrogen atmosphere. The resulting reaction mixture was stirred at 80° C. for 16 hours. After completion of the reaction, the reaction mixture was diluted with water (100 mL) and extracted with ethyl acetate (3×100 mL). Combined organic layer was dried over anhydrous Na₂SO₄, filtered and concentrated under reduced pressure to get crude residue which was purified by flash (50 g, 230-400 silica-gel mesh) column chromatography by using 0-100% ethyl acetate in hexane as eluent to afford tert-butyl 4-fluoro-4-[(6-hydroxy-4-oxo-quinazolin-3-yl)methyl]piperidine-1-carboxylate (3, 3.2 g, 8.39 mmol, 65% yield) as an off-white solid. UPLC-MS (ES−): m/z 376.1 [M−H]⁻.

### Step 2

Into a 250 mL single-neck round-bottom flask containing a well stirred solution of tert-butyl 4-fluoro-4-[(6-hydroxy-4-oxo-quinazolin-3-yl)methyl]piperidine-1-carboxylate (3, 3.7 g, 9.80 mmol) and 2,3,6-trifluorobenzonitrile (4, 11.76 mmol, 1.36 mL) in anhydrous THF (50 mL) was added Cs₂CO₃ (9.58 g, 29.41 mmol) at room temperature under nitrogen atmosphere. The resulting reaction mixture was stirred at room temperature for 16 hours. After completion of the reaction, the reaction mixture was diluted with water (50 mL) and extracted with ethyl acetate (3×50 mL). Combined organic layer was washed with saturated NaCl (50 mL), dried over anhydrous Na₂SO₄, filtered and concentrated under reduced pressure to afford a crude residue which was purified by flash (100-200 silica gel mesh) column chromatography using 0-100% ethyl acetate/hexane as an eluent to afford tert-butyl 4-[[6-(2-cyano-3,6-difluoro-phenoxy)-4-oxo-quinazolin-3-yl]methyl]-4-fluoro-piperidine-1-carboxylate (A-40, 4.4 g, 8.04 mmol, 82% yield) as an off-white solid. UPLC-MS (ES−): m/z 513.0 [M- H]⁻.

### Step 3

Into a 40 mL pressure vial containing a well stirred solution of tert-butyl 4-[[6-(2-cyano-3,6-difluoro-phenoxy)-4-oxo-quinazolin-3-yl]methyl]-4-fluoro-piperidine-1-carboxylate (5, 2.0 g, 3.89 mmol) and [methyl(sulfamoyl)amino]ethane (6, 966.92 mg, 7.00 mmol) in anhydrous N,N-dimethylformamide (20 mL) was added Cs₂CO₃ (4.43 g, 13.61 mmol) at room temperature under nitrogen atmosphere. The resulting reaction mixture was stirred at 60° C. for 16 hours. After completion of the reaction, the reaction mixture was diluted with water (100 mL) and extracted with ethyl acetate (2×100 mL). Combined organic layer was washed with saturated NaCl (100 mL), dried over anhydrous Na₂SO₄, filtered and concentrated under reduced pressure to afford a crude residue which was purified by flash (100-200 silica mesh, 50 g) column chromatography using 0-100% ethyl acetate/hexane as an eluent to afford tert-butyl 4-[[6-[2-cyano-3-[[ethyl(methyl)sulfamoyl]amino]-6-fluoro-phenoxy]-4-oxo-quinazolin-3-yl]methyl]-4-fluoro-piperidine-1-carboxylate (7, 1.6 g, 2.28 mmol, 59% yield) as an off-white solid. UPLC-MS (ES−): m/z 631.1 [M−H]⁻.

### Step 4

Into a 100 mL single-neck round-bottom flask containing a well stirred solution of tert-butyl 4-[[6-[2-cyano-3-[[ethyl(methyl)sulfamoyl]amino]-6-fluoro-phenoxy]-4-oxo-quinazolin-3-yl]methyl]-4-fluoro-piperidine-1-carboxylate (7, 800.00 mg, 1.26 mmol) in dichloromethane (20 mL) was added 4 M HCl in 1,4-dioxane (78.41 mmol, 6 mL) at 0° C. under nitrogen atmosphere. The resulting reaction mixture was stirred at room temperature for 3 hours. After completion of the reaction, the reaction mixture was concentrated under reduced pressure to afford crude 6-[2-cyano-3-[[ethyl(methyl)sulfamoyl]amino]-6-fluoro-phenoxy]-3-[(4-fluoro-4-piperidyl)methyl]-4-oxo-quinazoline (A-40, 0.850 g, 1.03 mmol, 82% yield, HCl salt) as an off-white solid. UPLC-MS (ES+): m/z 533.2 [M+H]⁺.

### Synthesis A41: Synthesis of 3-[(1S,5R)-3-azabicyclo[3.1.0]hexan-6-yl]-6-[2-cyano-3-[[ethyl(methyl)sulfamoyl]amino]-6-fluoro-phenoxy]-4-oxo-quinazoline (A-41)

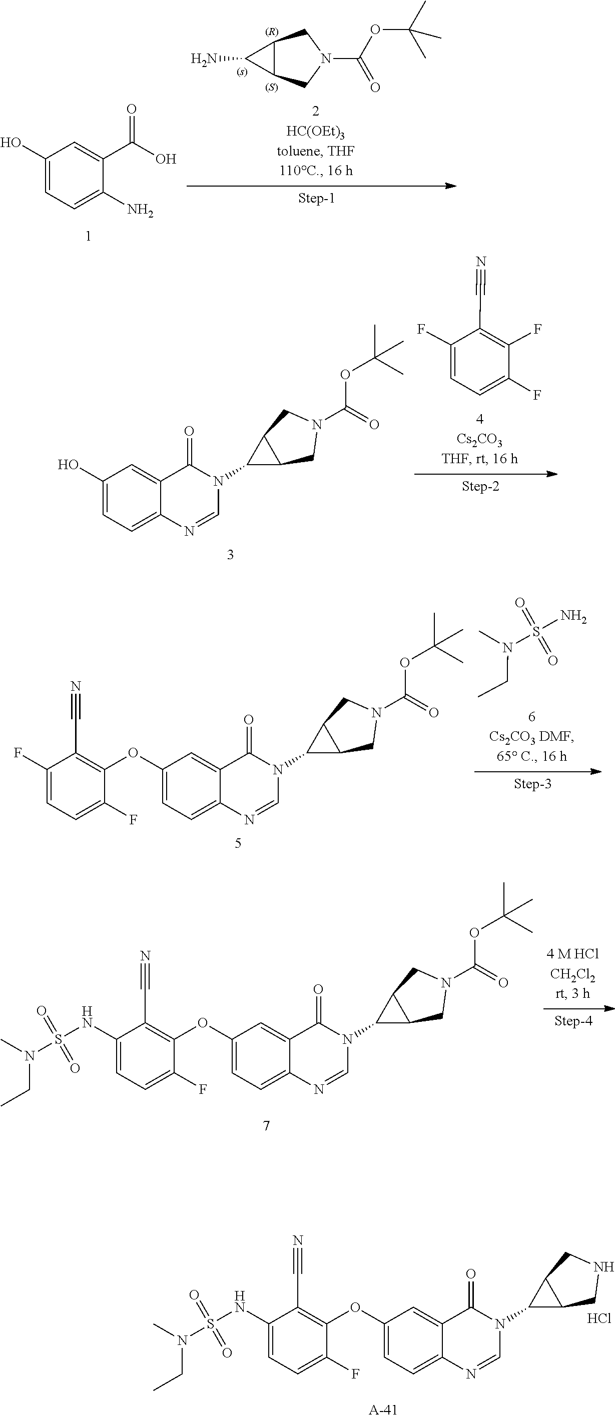

### Step 1

Into a 20 mL glass vial containing a well stirred solution of 2-amino-5-hydroxy-benzoic acid (1, 500 mg, 3.27 mmol) in Toluene (7 mL) and anhydrous THF (3 mL) was added tert-butyl (1S,5R)-6-amino-3-azabicyclo[3.1.0]hexane-3-carboxylate (2, 712.08 mg, 3.59 mmol) and Triethyl orthoformate (9.80 mmol, 1.63 mL) at room temperature under nitrogen atmosphere. After complete addition, the reaction mixture was stirred at 110° C. for 16 hours. After completion of the reaction, the reaction mixture was cooled to room temperature and poured into water (25 mL) and extracted with ethyl acetate (2×35 mL). Organic layers were combined and concentrated under reduced pressure to get crude, which was purified by flash column chromatography on silica-gel (230-400 mesh; 50 g) with 0-100% ethyl acetate in hexane to afford tert-butyl 4-[3-(2,4-dioxohexahydropyrimidin-1-yl)-5-fluoro-1-methyl-indazol-6-yl]-3-fluoro-3,6-dihydro-2H-pyridine-1-carboxylate (3, 685 mg, 1.97 mmol, 60% yield) as a yellow solid. UPLC-MS (ES+): m/z 344.33 [M+H]⁺.

### Step 2

Into a 50 mL single neck round bottom flask containing a well stirred solution of tert-butyl (1S,5R)-6-(6-hydroxy-4-oxo-quinazolin-3-yl)-3-azabicyclo[3.1.0]hexane-3-carboxylate (3, 680 mg, 1.98 mmol) in THF (9.93 mL) were added Cs₂CO₃ (1.94 g, 5.94 mmol) and 2,3,6-trifluorobenzonitrile (4, 2.38 mmol, 274.50 L) at room temperature. The reaction mixture was stirred at room temperature for 16 hours. After completion of the reaction, the reaction mixture was cooled to room temperature and poured into water (100 mL) and extracted with ethyl acetate (2×100 mL). Organic layers were evaporated under reduced pressure to get crude, which was purified by flash column chromatography on silica-gel (230-400 mesh; 50 g) with 0-100% ethyl acetate in hexane to afford tert-butyl (1S,5R)-6-[6-(2-cyano-3,6-difluoro-phenoxy)-4-oxo-quinazolin-3-yl]-3-azabicyclo[3.1.0]hexane-3-carboxylate (5, 800 mg, 1.58 mmol, 80% yield) as a yellow solid. UPLC-MS (ES+): m/z 481.33 [M+H]⁺.

### Step 3

Into a 100 mL two necked round bottomed flask containing a well-stirred solution of tert-butyl (1S,5R)-6-[6-(2-cyano-3,6-difluoro-phenoxy)-4-oxo-quinazolin-3-yl]-3-azabicyclo[3.1.0]hexane-3-carboxylate (5, 800 mg, 1.67 mmol) in N,N-dimethylformamide (12 mL) was added Cs₂CO₃ (1.63 g, 5.00 mmol) and [methyl(sulfamoyl)amino]ethane (6, 460.18 mg, 3.33 mmol) at ambient temperature under nitrogen atmosphere and the reaction mixture was stirred at 65° C. for 16 hours. After completion of the reaction, the reaction mixture was poured in cold water (100 mL), and extracted with ethyl acetate (2×100 mL), combined organic layer was washed saturated NaCl (20 mL) and dried over Na₂SO₄ concentrated under reduced pressure to afford a crude residue which was purified by flash column chromatography on silica-gel (230-400 mesh; 50 g) with 0-100% ethyl acetate in hexane to afford tert-butyl (1S,5R)-6-[6-[2-cyano-3-[[ethyl(methyl)sulfamoyl]amino]-6-fluoro-phenoxy]-4-oxo-quinazolin-3-yl]-3-azabicyclo[3.1.0]hexane-3-carboxylate (7, 350 mg, 579.22 mol, 35% yield) as an off-white solid. UPLC-MS (ES+): m/z 599.20 [M+H]⁺.

### Step 4

Into a 50 mL single neck round bottom flask containing a well stirred solution of tert-butyl (1S,5R)-6-[6-[2-cyano-3-[[ethyl(methyl)sulfamoyl]amino]-6-fluoro-phenoxy]-4-oxo-quinazolin-3-yl]-3-azabicyclo[3.1.0]hexane-3-carboxylate (7, 350 mg, 584.65 mol) in dichloromethane (2.79 mL) was added 4 M HCl in 1,4-dioxane (4 M, 1.46 mL) at 0° C. under nitrogen atmosphere. The reaction mixture was stirred at ambient temperature for 3 hours. After completion of the reaction, the reaction mixture was concentrated under reduced pressure to afford crude 3-[(1S,5R)-3-azabicyclo[3.1.0]hexan-6-yl]-6-[2-cyano-3-[[ethyl(methyl)sulfamoyl]amino]-6-fluoro-phenoxy]-4-oxo-quinazoline (A-41, 320 mg, 576.79 mol, 99% yield, HCl salt) as an off-white solid. UPLC-MS (ES+): m/z 499.20 [M+H]⁺.

### Synthesis A42: Synthesis of 7-[2-cyano-3-[[ethyl(methyl)sulfamoyl]amino]-6-fluoro-phenoxy]-2-(4-piperidylmethoxy)quinoxaline (A-42)

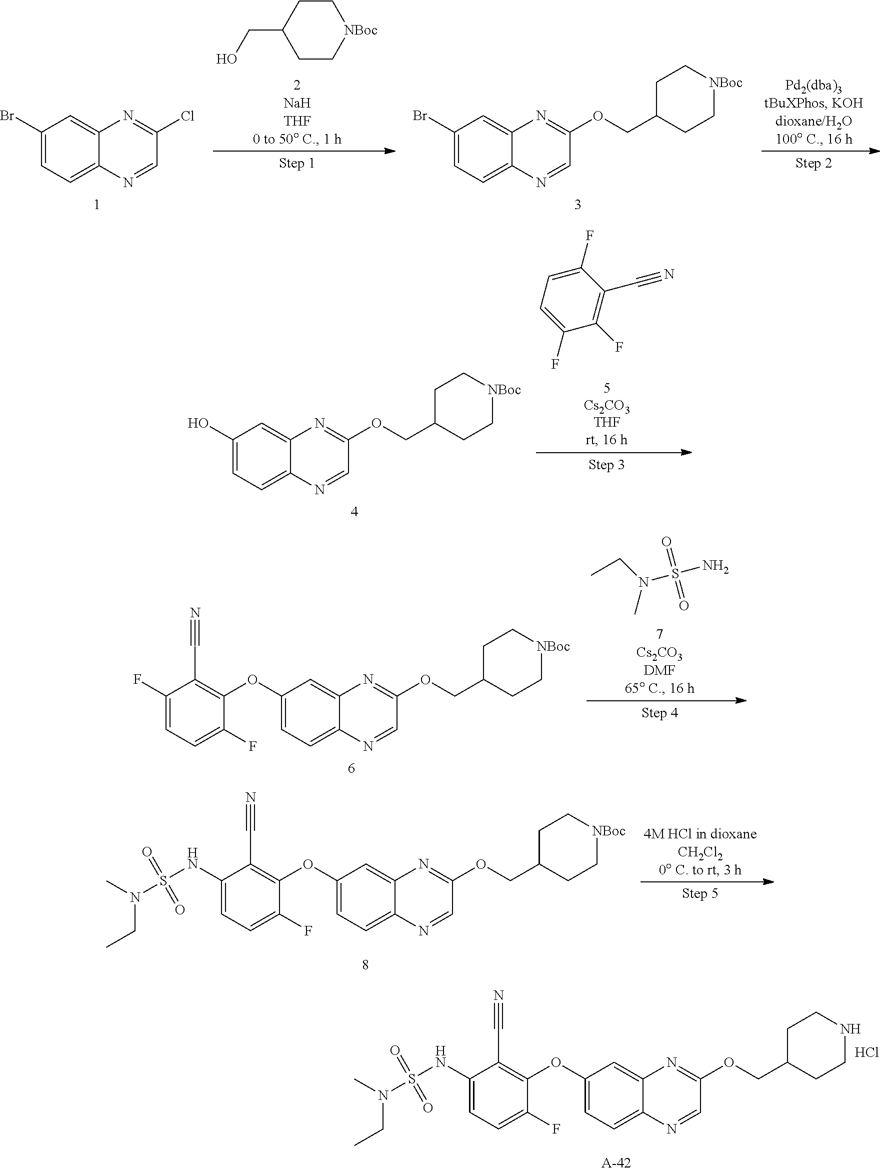

### Step 1

Into a 250 mL two neck round bottom flask containing a suspension of sodium hydride (2.05 g, 51.34 mmol, 60% purity) in THF (30 mL) was added tert-butyl 4-(hydroxymethyl)piperidine-1-carboxylate (2, 5.31 g, 24.64 mmol) in THF (70 mL) dropwise at 0° C. and stirred for 10 min. To the above mixture, 7-bromo-2-chloro-quinoxaline (1, 5 g, 20.53 mmol) in THF (30 mL) was added and the reaction mixture was allowed to heat at 50° C. for 1 h under nitrogen atmosphere. The reaction mixture was cooled to room temperature and quenched with saturated ammonium chloride solution (200 mL). Mixture was extracted with ethyl acetate (300 mL) and organic layer was separated, washed with H₂O (100 mL) and saturated NaCl (100 mL), dried over anhydrous Na₂SO₄, filtered and filtrate was evaporated to dryness under reduced pressure to obtain crude tert-butyl 4-[(7-bromoquinoxalin-2-yl)oxymethyl]piperidine-1-carboxylate (3, 8.5 g, 18.83 mmol, 92% yield) as a brown solid. UPLC-MS (ES+): m/z 423.9 [M+H]⁺.

### Step 2

Into a 100 mL sealed-tube containing a well-stirred solution of tert-butyl 4-[(7-bromoquinoxalin-2-yl)oxymethyl]piperidine-1-carboxylate (3, 3 g, 7.10 mmol) in anhydrous 1,4-dioxane (30 mL) and water (15 mL) was added KOH (398.56 mg, 7.10 mmol) at room temperature under nitrogen atmosphere and resulting mixture was degassed with nitrogen gas into for 10 min. Subsequently, ^{t-}BuXPhos (150.60 mg, 355.18 μmol) and tris(dibenzylideneacetone) dipalladium(0) (130.10 mg, 142.07 μmol) was added and reaction mixture was degassed for additional 10 min and stirred at 100° C. for 16 hours. After completion of the reaction, the reaction mixture was cooled to room temperature and poured into water (150 mL) and extracted with ethyl acetate (2×350 mL). Combined organic layers were washed with saturated NaCl (150 mL), dried over anhydrous Na₂SO₄, filtered and filtrate was concentrated under reduced pressure to get a crude residue which was purified by flash silica-gel (230-400 mesh, 100 g) column chromatography eluting with 0-100% ethyl acetate/hexane to afford tert-butyl 4-[(7-hydroxyquinoxalin-2-yl)oxymethyl]piperidine-1-carboxylate (4, 2.5 g, 6.29 mmol, 88% yield) as a brown solid. UPLC-MS (ES+): m/z 360.1 [M+H]⁺.

### Step 3

Into a 100 mL single-neck round-bottom flask containing a well-stirred solution tert-butyl 4-[(7-hydroxyquinoxalin-2-yl)oxymethyl]piperidine-1-carboxylate (4, 2.5 g, 6.96 mmol) in anhydrous THF (60 mL) was added Cs₂CO₃ (6.80 g, 20.87 mmol) and 2,3,6-trifluorobenzonitrile (5, 8.35 mmol, 964.13 μL) under nitrogen atmosphere. After complete addition, the reaction mixture was allowed to stir at room temp for 16 h under nitrogen atmosphere. After completion of reaction, the reaction mixture was diluted with ethyl acetate (200 mL) and washed successively with H₂O (100 mL) and saturated NaCl (100 mL). Organic layer was separated and dried over anhydrous Na₂SO₄, filtered and filtrate was evaporated to dryness under reduced pressure to obtain crude residue, which was purified by flash (50 g, silica-gel 230-400 mesh) column with 0-100% ethyl acetate/hexane while desired product eluting at 30-40% of mobile phase to afford tert-butyl 4-[[7-(2-cyano-3,6-difluoro-phenoxy)quinoxalin-2-yl]oxymethyl]piperidine-1-carboxylate (6, 2.7 g, 5.43 mmol, 78% yield) as an off-white solid. UPLC-MS (ES+): m/z 441.0 [M+H−^{t}Bu]⁺.

### Step 4

Into a 250 mL single-neck round-bottom flask containing a well-stirred solution tert-butyl 4-[[7-(2-cyano-3,6-difluoro-phenoxy)quinoxalin-2-yl]oxymethyl]piperidine-1-carboxylate (6, 2.7 g, 5.44 mmol) in anhydrous N,N-dimethylformamide (50 mL) was added methyl(sulfamoyl)amino]ethane (7, 1.50 g, 10.88 mmol) and Cs₂CO₃ (5.32 g, 16.31 mmol) at room temperature under nitrogen atmosphere. Reaction mixture was stirred at 65° C. under nitrogen atmosphere for 16 hours. After completion of reaction, reaction mixture was diluted with water (200 mL) and precipitated solid was filtered. Filtrate was and extracted with ethyl acetate (2×250 mL) and combined organic layer was washed with saturated NaCl (2×150 mL), dried over anhydrous Na₂SO₄, filtered and filtrate was evaporated to dryness under reduced pressure to obtain crude tert-butyl 4-[[7-[2-cyano-3-[[ethyl(methyl)sulfamoyl]amino]-6-fluoro-phenoxy]quinoxalin-2-yl]oxymethyl]piperidine-1-carboxylate (8, 3.0 g, 4.30 mmol, 79% yield) as a brown thick liquid. UPLC-MS (ES−): m/z 613.2 [M−H]⁻.

### Step 5

Into a 100 mL single-neck round-bottom flask containing a well-stirred solution of tert-butyl 4-[[7-[2-cyano-3-[[ethyl(methyl)sulfamoyl]amino]-6-fluoro-phenoxy]quinoxalin-2-yl]oxymethyl]piperidine-1-carboxylate (8, 3 g, 4.29 mmol) in anhydrous dichloromethane (41.76 mL) was added 4M HCl in 1,4-dioxane (12.88 mL) at 0° C. and resulting mixture was stirred at room temperature under nitrogen atmosphere for 3 hours. After completion of reaction, the reaction mixture was concentrated under reduced pressure to get a crude residue which washed with methyl tert-butyl ether (100 mL) to get the 7-[2-cyano-3-[[ethyl(methyl)sulfamoyl]amino]-6-fluoro-phenoxy]-2-(4-piperidylmethoxy)quinoxaline (A-42, 2.3 g, 3.98 mmol, 93% yield, HCl salt) as a brown thick liquid. UPLC-MS (ES+): m/z 515.4 [M+H]⁺.

### Synthesis A43: Synthesis of 7-[2-cyano-3-[[ethyl(methyl)sulfamoyl]amino]-6-fluoro-phenoxy]-2-[(4-hydroxy-4-piperidyl)methoxy]quinoxaline (A-43)

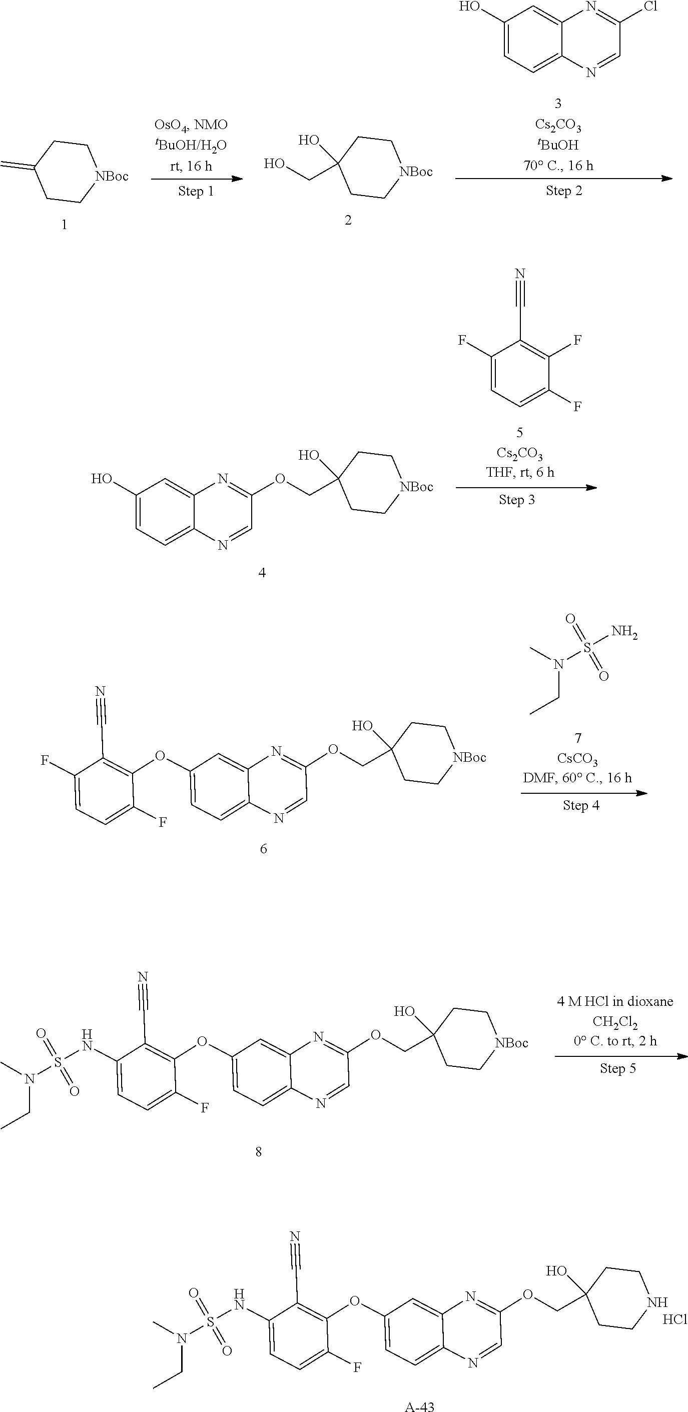

### Step 1

Into a 500 mL single-neck round-bottom flask containing a well stirred-solution of tert-butyl 4-methylenepiperidine-1-carboxylate (1, 6 g, 30.41 mmol) in t-BuOH (80 mL) and water (20 mL) was added osmium tetroxide 4% in water (304.15 mol, 4% purity, 393.87 μL) followed by N-methylmorpholine N-oxide (10.69 g, 91.24 mmol) at 0° C. Resulting reaction mixture was stirred at room temperature for 16 hours. After completion of the reaction as indicated by TLC, reaction mixture was quenched with solid Na₂SO₃ (6 g) and concentrated under reduced pressure below 40° C. to get crude residue, which was diluted with water (200 mL) and extracted with ethyl acetate (2×300 mL). The combined organic layers were dried over anhydrous Na₂SO₄, filtered and concentrated under reduced pressure to afford tert-butyl 4-hydroxy-4-(hydroxymethyl)piperidine-1-carboxylate (2, 6 g, 25.90 mmol, 85% yield) as an off-white solid. UPLC-MS (ES+): m/z 132.2 [M−Boc+H]⁺.

### Step 2

Into a 100 mL sealed-tube containing a well-stirred solution of 3-chloroquinoxalin-6-ol (3, 1 g, 5.54 mmol) and tert-butyl 4-hydroxy-4-(hydroxymethyl)piperidine-1-carboxylate (2, 1.92 g, 8.31 mmol) in t-BuOH (20 mL) was added Cs₂CO₃ (10.83 g, 33.22 mmol) at room temperature. Resulting reaction mixture was stirred at 70° C. for 16 hours. After completion, the reaction mixture was concentrated under reduced pressure to get crude residue, which was diluted with water (100 mL) and extracted with ethyl acetate (2×200 mL). Combined organic layer was dried over anhydrous Na₂SO₄, filtered and concentrated under reduced pressure to get crude, which was purified by flash silica gel (100-200 mesh, 50 g) column chromatography using 0-100% ethyl acetate/hexane as an eluent to afford tert-butyl 4-hydroxy-4-[(7-hydroxyquinoxalin-2-yl)oxymethyl]piperidine-1-carboxylate (4, 750 mg, 1.97 mmol, 36% yield) as an off-white solid. UPLC-MS (ES+): m/z 320.0 [M−^{t}Bu+H]⁺.

### Step 3

Into a 100 mL single-neck round-bottom flask containing a well-stirred solution of tert-butyl 4-hydroxy-4-[(7-hydroxyquinoxalin-2-yl)oxymethyl]piperidine-1-carboxylate (4, 1.5 g, 4.00 mmol) in THF (30 mL) were added Cs₂CO₃ (3.91 g, 11.99 mmol) and 2,3,6-trifluorobenzonitrile (5, 4.79 mmol, 553.83 μL) at 0° C. Resulting reaction mixture was stirred at room temperature for 6 hours. After completion of the reaction, the reaction mixture was diluted with water (100 mL) and extracted with ethyl acetate (2×150 mL). Combined organic layer was dried over anhydrous Na₂SO₄, filtered and concentrated under reduced pressure to get crude residue, which was purified by flash silica gel (100-200 mesh, 50 g) column chromatography using 0-100% ethyl acetate/hexane as an eluent to afford tert-butyl 4-[[7-(2-cyano-3,6-difluoro-phenoxy)quinoxalin-2-yl]oxymethyl]-4-hydroxy-piperidine-1-carboxylate (6, 1.7 g, 3.31 mmol, 83% yield) as an off-white solid. UPLC-MS (ES+): m/z 457.2 [M-tBu+H]⁺.

### Step 4

Into a 20 mL glass vial with pressure released cap containing a well-stirred solution of tert-butyl 4-[[7-(2-cyano-3,6-difluoro-phenoxy)quinoxalin-2-yl]oxymethyl]-4-hydroxy-piperidine-1-carboxylate (6, 400 mg, 779.70 mol) in N,N-dimethylformamide (10 mL) were added Cs₂CO₃ (762.13 mg, 2.34 mmol) and [methyl(sulfamoyl)amino]ethane (7, 161.62 mg, 1.17 mmol) at room temperature and stirred at 60° C. for 16 hours. After completion of the reaction, the reaction mixture was diluted with water (100 mL) and extracted with ethyl acetate (2×100 mL). Combined organic layer was dried over anhydrous Na₂SO₄, filtered and concentrated under reduced pressure to get a crude residue, which was purified by flash silica gel (100-200 mesh, 50 g) column chromatography using 0-100% ethyl acetate/hexane as an eluent while desired product was eluted at 45-50% of mobile phase to afford tert-butyl 4-[[7-[2-cyano-3-[[ethyl(methyl)sulfamoyl]amino]-6-fluoro-phenoxy]quinoxalin-2-yl]oxymethyl]-4-hydroxy-piperidine-1-carboxylate (8, 350 mg, 539.02 μmol, 69% yield) as a light brown thick gum. UPLC-MS (ES+): m/z 531.0 [M−Boc+H]⁺.

### Step 5

Into a 100 mL single-neck round-bottom flask containing a well-stirred solution of tert-butyl 4-[[7-[2-cyano-3-[[ethyl(methyl)sulfamoyl]amino]-6-fluoro-phenoxy]quinoxalin-2-yl]oxymethyl]-4-hydroxy-piperidine-1-carboxylate (8, 700 mg, 1.08 mmol) in dichloromethane (15 mL) was added 4M hydrogen chloride in 1,4-dioxane (4 M, 2.69 mL) at 0° C. Resulting reaction mixture was stirred at room temperature for 2 hours. After completion of the starting material as indicated by TLC, reaction mixture was concentrated directly under reduced pressure to afford 7-[2-cyano-3-[[ethyl(methyl)sulfamoyl]amino]-6-fluoro-phenoxy]-2-[(4-hydroxy-4-piperidyl)methoxy]quinoxaline (A-43, 600 mg, 950.00 mol, 88% yield, HCl salt) as an off-white solid. UPLC-MS (ES+): m/z 531.0 [M+H]⁺.

### Synthesis A44: Synthesis of 7-[2-cyano-3-[[ethyl(methyl)sulfamoyl]amino]-6-fluoro-phenoxy]-2-[(4-fluoro-4-piperidyl)methoxy]quinoxaline (A-44)

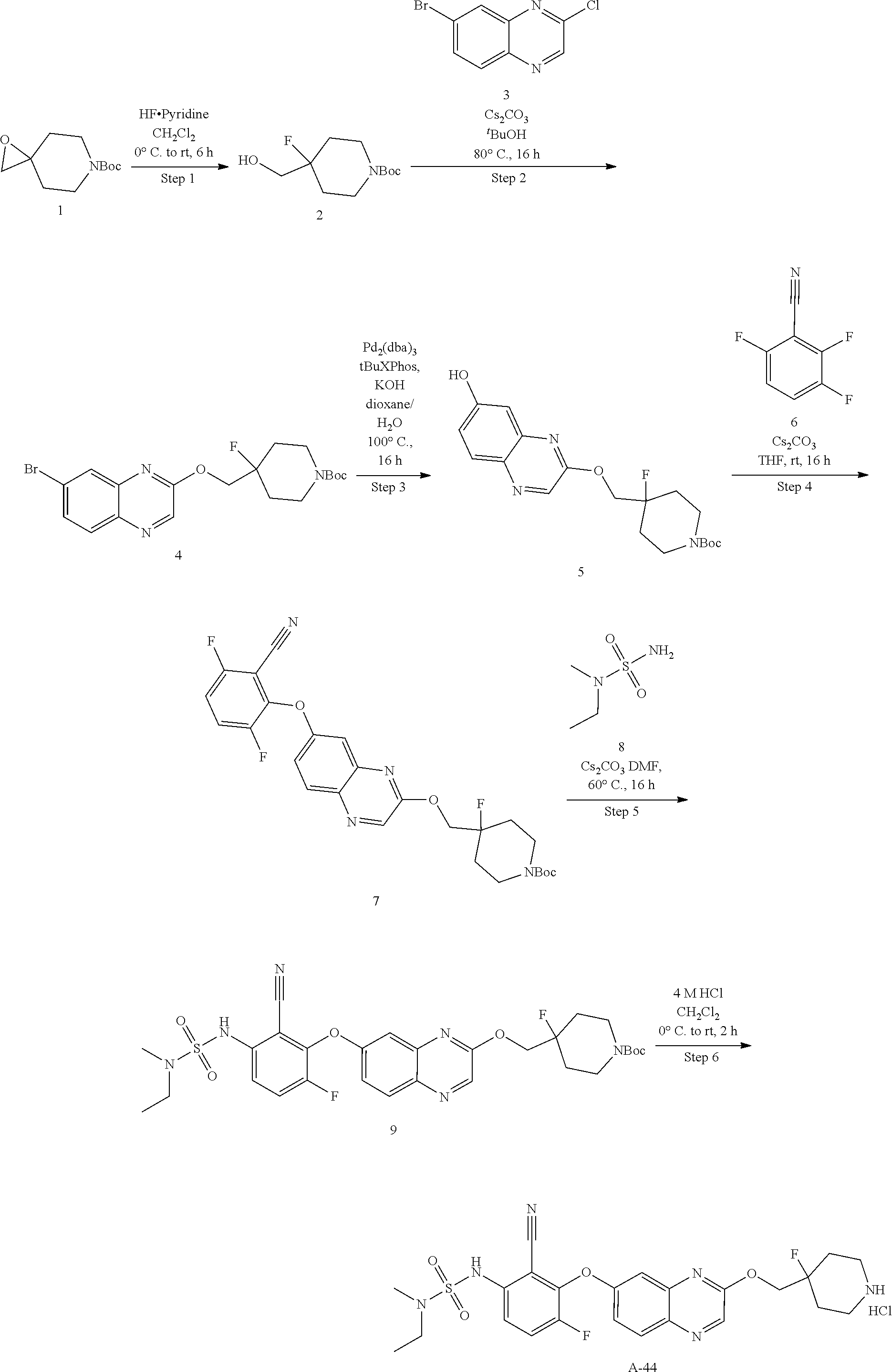

### Step 1

Into a 500 mL two-neck round-bottom flask containing a well-stirred solution of tert-butyl 1-oxa-6-azaspiro[2.5]octane-6-carboxylate (1, 5.0 g, 23.44 mmol) in dichloromethane (75 mL) was added dropwise pyridine;hydrofluoride (25.79 mmol, 2.24 mL) at 0° C. After addition the suspension was stirred at room temperature under nitrogen atmosphere for 6 hours. After completion of the reaction, the reaction mixture was diluted with dichloromethane (100 mL) and ice-water (80 mL) stirred for 15 min. The pH of the reaction mixture was adjusted to ~7 using 10% NaHCO₃ solution. The layers were separated, and aqueous layer was extracted with dichloromethane (100 mL). Combined organic layers were dried over anhydrous Na₂SO₄, filtered and filtrate was concentrated under reduced pressure to afford crude which was purified by flash silica-gel (230-400 mesh; 100 g) column with 0-100% ethyl acetate/hexane to afford tert-butyl 4-fluoro-4-(hydroxymethyl)piperidine-1-carboxylate (2, 1.6 g, 6.65 mmol, 28% yield) as a colorless liquid. UPLC-MS (ES+): m/z 134.2 [M+H−Boc]⁺.

### Step 2

Into a 100 mL single-neck round-bottom flask containing a well-stirred solution of 7-bromo-2-chloro-quinoxaline (3, 1.1 g, 4.52 mmol) and tert-butyl 4-fluoro-4-(hydroxymethyl)piperidine-1-carboxylate (2, 1.58 g, 6.78 mmol) in t-BuOH (22 mL) was added Cs₂CO₃ (7.36 g, 22.59 mmol) at room temperature and the resulting suspension was stirred at 80° C. for 16 hours. After completion of the reaction, the reaction mixture was allowed to room temperature and quenched with cold-water (50 mL) and diluted with ethyl acetate (100 mL). Organic layer was separated and dried over anhydrous Na₂SO₄, filtered, filtrate was concentrated under reduced pressure to afford crude which was purified by flash silica-gel (60-120 mesh) column with 0-100% ethyl acetate/hexane while compound was eluting at 30-40% of the mobile phase to afford tert-butyl 4-[(7-bromoquinoxalin-2-yl)oxymethyl]-4-fluoro-piperidine-1-carboxylate (4, 1.9 g, 4.06 mmol, 90% yield) as an off-white solid. UPLC-MS (ES+): m/z 384.0 [M+H−^{t}Bu]⁺.

### Step 3

Into a 50 mL sealed-tube containing a well stirred solution of H₂O (8 mL), was added KOH (605.31 mg, 10.79 mmol, 296.14 L) at 10° C. and stirred for 10 min. After 10 min of stirring, 1,4-dioxane (22 mL) was added followed by tert-butyl 4-[(7-bromoquinoxalin-2-yl)oxymethyl]-4-fluoro-piperidine-1-carboxylate (4, 1.9 g, 4.32 mmol, No Salt) and ^{t-}BuXPhos (183.24 mg, 431.52 mol). The reaction mixture was purged with nitrogen for 10 min. Pd₂(dba)₃ (395.15 mg, 431.52 mol) was added and stirred at 100° C. for 16 hours. After completion of the reaction, the reaction mixture was allowed to room temperature. The pH of the reaction mixture was adjusted to ~3 using 1.5N HCl (15 mL) solution. Aqueous layer was extracted with ethyl acetate (2×40 mL) and the combined organic layers were washed with saturated NaCl (50 mL), dried over anhydrous Na₂SO₄, filtered and filtrate was concentrated under reduced pressure to afford crude which was purified by flash silica-gel (60-120 mesh) column with 0-100% ethyl acetate/hexane while desired product eluting at 35-40% of the mobile phase to afford tert-butyl 4-fluoro-4-[(7-hydroxyquinoxalin-2-yl)oxymethyl]piperidine-1-carboxylate (5, 1.1 g, 2.80 mmol, 65% yield) as an off-white solid. UPLC-MS (ES−): m/z 376.2 [M−H]⁻.

### Step 4

Into a 50 mL single-neck round-bottom flask containing a well-stirred solution of tert-butyl 4-fluoro-4-[(7-hydroxyquinoxalin-2-yl)oxymethyl]piperidine-1-carboxylate (5, 1.2 g, 3.18 mmol) in THF (12.00 mL) were added 2,3,6-trifluorobenzonitrile (6, 4.77 mmol, 550.91 L) and Cs₂CO₃ (3.11 g, 9.54 mmol) at room temperature and the resulting suspension was stirred at room temperature for 16 h under nitrogen atmosphere. After completion of the reaction, the reaction mixture was diluted with water (20 mL) and extracted with dichloromethane (2×50 mL). Combined organic layers were dried over anhydrous Na₂SO₄, filtered and the filtrate was concentrated under reduced pressure to get a crude residue which was purified by flash silica-gel (60-120 mesh) column with 0-100% ethyl acetate/hexane while compound was eluting at 40-50% of the mobile phase to afford tert-butyl-4-[[7-(2-cyano-3,6-difluoro-phenoxy)quinoxalin-2-yl]oxymethyl]-4-fluoro-piperidine-1-carboxylate (7, 1.0 g, 1.89 mmol, 59% yield) as a pale yellow solid. UPLC-MS (ES+): m/z 459.3 [M+H−^{t}Bu]⁺.

### Step 5

Into a 50 mL single-neck round-bottom flask containing a well-stirred solution of tert-butyl 4-[[7-(2-cyano-3,6-difluoro-phenoxy)quinoxalin-2-yl]oxymethyl]-4-fluoro-piperidine-1-carboxylate (7, 1.0 g, 1.94 mmol) and [methyl(sulfamoyl)amino]ethane (8, 537.18 mg, 3.89 mmol) in N,N-dimethylformamide (10 mL) was added Cs₂CO₃ (1.58 g, 4.86 mmol) at room temperature and the resulting suspension was stirred at 60° C. for 16 hours. After completion of the reaction, the reaction mixture was diluted with water (60 mL) and extracted with ethyl acetate (2×60 mL). Combined organic phases were dried (anhydrous Na₂SO₄), filtered and the filtrate was concentrated under reduced pressure to get a crude residue which was purified by flash silica-gel (230-400 mesh) column with 0-100% ethyl acetate/hexane to afford tert-butyl 4-[[7-[2-cyano-3-[[ethyl(methyl)sulfamoyl]amino]-6-fluoro-phenoxy]quinoxalin-2-yl]oxymethyl]-4-fluoro-piperidine-1-carboxylate (9, 700 mg, 1.01 mmol, 52% yield) as a pale yellow solid. UPLC-MS (ES−): m/z 533.5 [M−Boc]⁻.

### Step 6

Into a 50 mL single-neck round-bottom flask containing a well-stirred solution of tert-butyl 4-[[7-[2-cyano-3-[[ethyl(methyl)sulfamoyl]amino]-6-fluoro-phenoxy]quinoxalin-2-yl]oxymethyl]-4-fluoro-piperidine-1-carboxylate (9, 350 mg, 553.20 mol) in dichloromethane (4 mL) was added HCl, 4M in 1,4-dioxane (4 M, 2 mL) at 0° C. The resulting mixture was stirred at room temperature for 30 min. After completion of the reaction, the reaction mixture was concentrated under reduced pressure to afford 7-[2-cyano-3-[[ethyl(methyl)sulfamoyl]amino]-6-fluoro-phenoxy]-2-[(4-fluoro-4-piperidyl)methoxy]quinoxaline (A-44, 290 mg, 433.20 mol, 78% yield, HCl salt) as a brown gum. Crude was taken to the next step without further purification. UPLC-MS (ES+): m/z 533.5 [M+H]⁺.

### Synthesis A45: Synthesis of 2-(7-azaspiro[3.5]nonan-2-yloxy)-7-[2-cyano-3-[[ethyl(methyl)sulfamoyl]amino]-6-fluoro-phenoxy]quinoxaline (A-45)

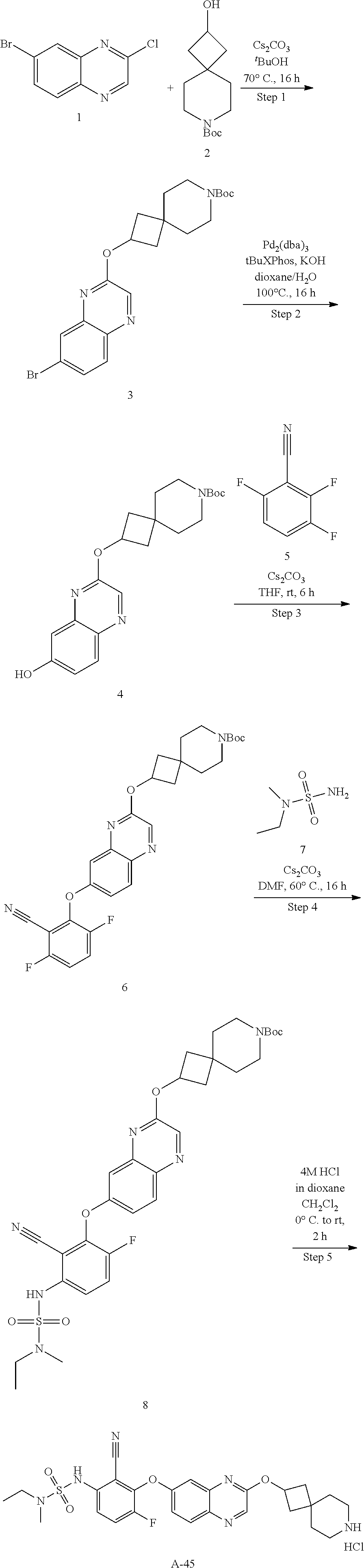

### Step 1

Into a 250 mL double-neck round-bottom flask containing a well-stirred solution of 7-bromo-2-chloro-quinoxaline (1, 2.5 g, 10.27 mmol) in ^{t}BuOH (40 mL) were added Cs₂CO₃ (16.73 g, 51.34 mmol) and tert-butyl 2-hydroxy-7-azaspiro[3.5]nonane-7-carboxylate (2, 2.97 g, 12.32 mmol) at room temperature and stirred at 70° C. for 16 hours. After completion of the reaction, the reaction mixture was concentrated under reduced pressure, diluted with water (100 mL) and extracted with ethyl acetate (2×200 mL). Combined organic layer was dried over anhydrous Na₂SO₄, filtered and concentrated under reduced pressure to get crude residue, which was purified by flash silica gel (100-200 mesh, 100 g) column chromatography using 0-100% ethyl acetate/hexane as an eluent to afford tert-butyl 2-(7-bromoquinoxalin-2-yl)oxy-7-azaspiro[3.5]nonane-7-carboxylate (3, 4 g, 8.85 mmol, 86% yield) as an off-white solid. UPLC-MS (ES+): m/z 394.0 [M−^{t}Bu+H]⁺.

### Step 2

Into a 40 mL glass vial with pressure released cap containing a well-stirred solution of KOH (625.74 mg, 11.15 mmol, 306.13 L) in Water (3 mL) and 1,4 dioxane (12 mL) cooled to 10° C. were added tert-butyl 2-(7-bromoquinoxalin-2-yl)oxy-7-azaspiro[3.5]nonane-7-carboxylate (3, 2 g, 4.46 mmol) and tBuXPhos (189.42 mg, 446.08 mol) at same temperature, reaction solution was purged with nitrogen for 15 minutes. Finally, Pd₂dba₃ (408.48 mg, 446.08 mol) was added at room temperature. The resulting reaction mixture was stirred at 100° C. for 16 hours. After completion of the reaction, the reaction mixture was diluted with water (50 mL) and extracted with ethyl acetate (2×100 mL). Combined organic layer was dried over anhydrus Na₂SO₄, filtered and concentrated under reduced pressure to get crude residue which was purified by flash silica gel (100-200 mesh, 100 g) column chromatography using 0-100% ethyl acetate/hexane as an eluent to afford tert-butyl 2-(7-hydroxyquinoxalin-2-yl)oxy-7-azaspiro[3.5]nonane-7-carboxylate (4, 1.3 g, 2.69 mmol, 60% yield) as an off-white solid. UPLC-MS (ES+): m/z 330.0 [M-tBu+H]⁺.

### Step 3

Into a 250 mL single-neck round-bottom flask containing a well stirred solution of tert-butyl 2-(7-hydroxyquinoxalin-2-yl)oxy-7-azaspiro[3.5]nonane-7-carboxylate (4, 3 g, 6.23 mmol) in THF (50 mL) were added Cs₂CO₃ (6.09 g, 18.68 mmol) and 2,3,6-trifluorobenzonitrile (5, 7.47 mmol, 863.05 μL) at 0° C. Resulting reaction mixture was stirred at room temperature for 6 hours. After completion of the reaction, the reaction mixture was diluted with water (100 mL) and extracted with ethyl acetate (2×150 mL). Combined organic layer was dried over anhydrous Na₂SO₄, filtered and concentrated under reduced pressure to get crude residue, which was purified by flash silica gel (100-200 mesh, 100 g) column chromatography using 0-100% ethyl acetate/hexane as an eluent to afford tert-butyl 2-[7-(2-cyano-3,6-difluoro-phenoxy)quinoxalin-2-yl]oxy-7-azaspiro[3.5]nonane-7-carboxylate (6, 3.1 g, 5.81 mmol, 93% yield) as an off-white solid. UPLC-MS (ES+): m/z 467.0 [M-tBu+H]⁺

### Step 4

Into a 20 mL glass vial with pressure released cap containing a well-stirred solution of tert-butyl 2-[7-(2-cyano-3,6-difluoro-phenoxy)quinoxalin-2-yl]oxy-7-azaspiro[3.5]nonane-7-carboxylate (6, 800 mg, 1.50 mmol) in N,N-dimethylformamide (10 mL) were added Cs₂CO₃ (1.47 g, 4.50 mmol) and [methyl(sulfamoyl)amino]ethane (7, 311.00 mg, 2.25 mmol) at room temperature. The resulting reaction mixture was stirred at 60° C. for 16 hours. After completion of the reaction, the reaction mixture was diluted with water (100 mL) and extracted by ethyl acetate (2×150 mL). Combined organic layer was dried over anhydrous Na₂SO₄, filtered and concentrated under reduced pressure to get a crude residue, which was purified by flash silica gel (100-200 mesh, 50 g) column chromatography using 0-100% ethyl acetate/hexane as an eluentto afford tert-butyl 2-[7-[2-cyano-3-[[ethyl(methyl)sulfamoyl]amino]-6-fluoro-phenoxy]quinoxalin-2-yl]oxy-7-azaspiro[3.5]nonane-7-carboxylate (8, 750 mg, 1.07 mmol, 72% yield) as a light brown thick gum. UPLC-MS (ES+): m/z 585.1 [M−^{t}Bu+H]⁺.

### Step 5

Into a 100 mL single-neck round-bottom flask containing a well-stirred solution of tert-butyl 2-[7-[2-cyano-3-[[ethyl(methyl)sulfamoyl]amino]-6-fluoro-phenoxy]quinoxalin-2-yl]oxy-7-azaspiro[3.5]nonane-7-carboxylate (8, 800 mg, 1.15 mmol) in dichloromethane (10 mL) was added 4 M hydrogen chloride in 1,4-dioxane (4 M, 2.87 mL) at 0° C. Resulting reaction mixture was stirred at room temperature for 2 hours. After completion of the starting material as indicated by TLC, reaction mixture was concentrated directly under reduced pressure to afford 2-(7-azaspiro[3.5]nonan-2-yloxy)-7-[2-cyano-3-[[ethyl(methyl)sulfamoyl]amino]-6-fluoro-phenoxy]quinoxaline (A-45, 650 mg, 1.09 mmol, 95% yield, HCl salt) as an off-white solid. UPLC-MS (ES+): m/z 541.5 [M+H]⁺.

### Synthesis A46: Synthesis of 2-(7-azaspiro[3.5]nonan-2-yl)-7-[2-cyano-3-[[ethyl(methyl)sulfamoyl]amino]-6-fluoro-phenoxy]quinoxaline (A-46)

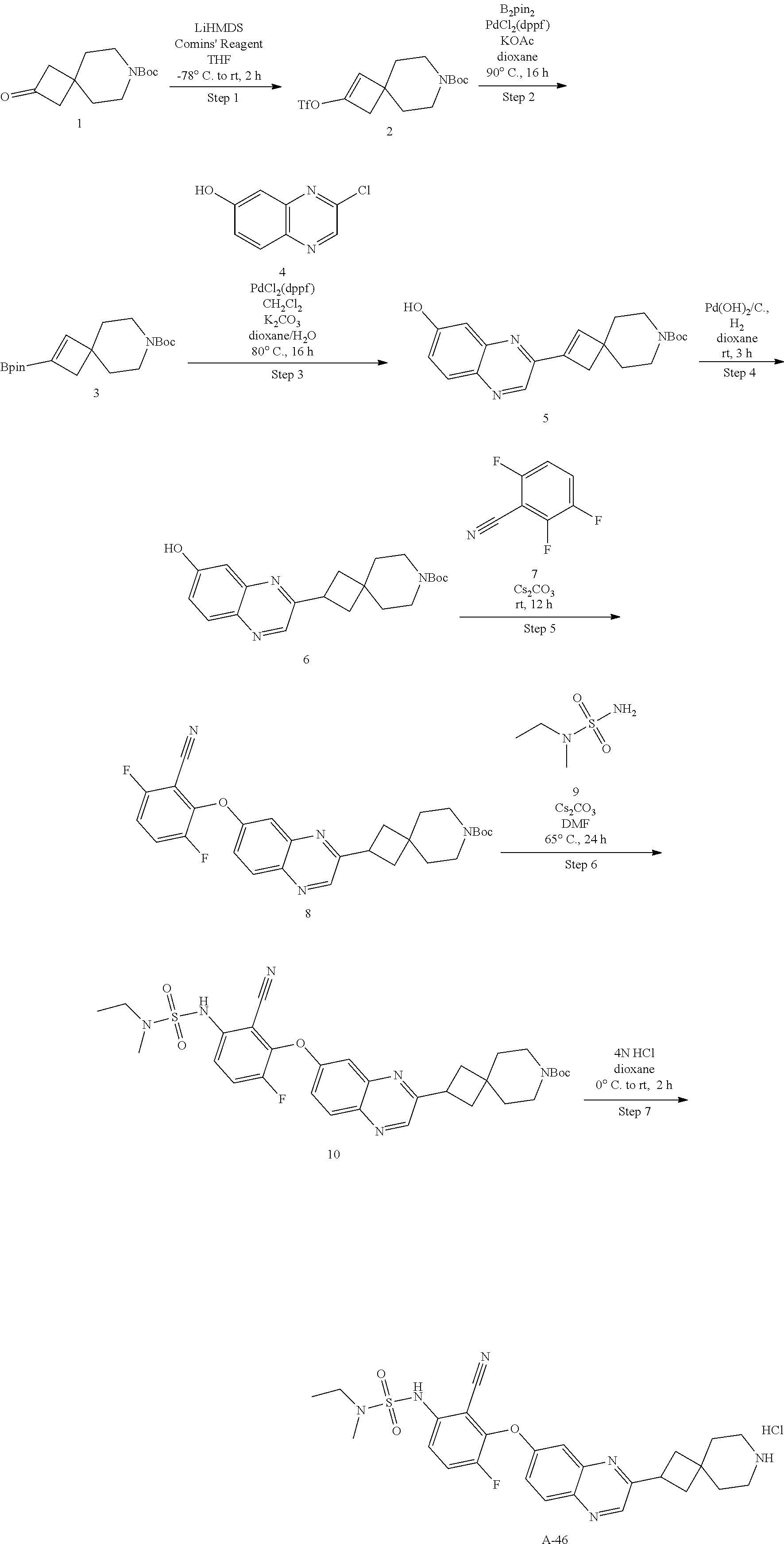

### Step 1

Into a 500 mL three-neck round-bottom flask containing a well stirred solution of tert-butyl 2-oxo-7-azaspiro[3.5]nonane-7-carboxylate (1, 5.0 g, 20.89 mmol) in anhydrous THF (60 mL) was added LiHMDS (1.0 M in THF, 50.14 mL) dropwise at −78° C. under nitrogen atmosphere and stirred at same temperature for 1 hour. A freshly prepared solution of N-(5-chloro-2-pyridyl)-1,1,1-trifluoro-N-(trifluoromethylsulfonyl)methanesulfonamide (12.31 g, 31.34 mmol) in anhydrous THF (60 mL) was added to the above reaction mixture at −78° C. under nitrogen atmosphere. After addition, reaction mixture was allowed to cool to room temperature and stirred for 2 hours. The reaction mixture was quenched with saturated NH₄C₁ solution (100 mL) at 0° C. and diluted with ethyl acetate (150 mL). The organic phase was separated, and aqueous phase was extracted with ethyl acetate (2×80 mL). The combined organic phases were dried over anhydrous Na₂SO₄, filtered, and concentrated under reduced pressure to get a crude residue which was purified by flash silica-gel (230-400 mesh) column using 0-100% ethyl acetate/hexane as an eluent to afford tert-butyl 2-(trifluoromethylsulfonyloxy)-7-azaspiro[3.5]non-2-ene-7-carboxylate (2, 3.5 g, 9.24 mmol, 44% yield) as an off-white solid. UPLC-MS (ES+): m/z 272.0 [M+H−Boc]⁺.

### Step 2

Into a 250 mL single-neck round-bottom flask containing a well-stirred solution of tert-butyl 2-(trifluoromethylsulfonyloxy)-7-azaspiro[3.5]non-2-ene-7-carboxylate (2, 3.5 g, 9.42 mmol) and 4,4,5,5-tetramethyl-2-(4,4,5,5-tetramethyl-1,3,2-dioxaborolan-2-yl)-1,3,2-dioxaborolane (3.11 g, 12.25 mmol) in anhydrous 1,4-dioxane (35 mL) was added potassium acetate (2.31 g, 23.56 mmol) and mixture was degassed by bubbling nitrogen gas for 5 minutes. Subsequently, bis(1-diphenylphosphanylcyclopenta-2,4-dien-1-yl)iron dichloropalladium dichloromethane complex (769.64 mg, 942.45 mol) was added to the reaction mixture and reaction mixture was stirred at 90° C. under nitrogen atmosphere for 16 hours. After the completion of the reaction, the reaction mixture was allowed to room temperature and filtered through a pad of Celite and Celite pad was washed with ethyl acetate (100 mL). Filtrate was concentrated under reduced pressure to get a crude residue, which was purified by flash silica-gel (230-400 mesh) column using 0-100% ethyl acetate/hexane as an eluent to afford mixture of tert-butyl 2-(4,4,5,5-tetramethyl-1,3,2-dioxaborolan-2-yl)-7-azaspiro[3.5]non-2-ene-7-carboxylate (3, 2.5 g, 3.94 mmol, 42% yield) as an off-white solid. UPLC-MS (ES+): m/z 250.2 [M+H−Boc]⁺.

### Step 3

Into a 250 mL single-neck round bottom flask containing a well-stirred solution of 3-chloroquinoxalin-6-ol (4, 1.2 g, 6.64 mmol) and tert-butyl 2-(4,4,5,5-tetramethyl-1,3,2-dioxaborolan-2-yl)-7-azaspiro[3.5]non-2-ene-7-carboxylate (3, 2.5 g, 7.31 mmol) in 1,4-dioxane (24 mL) and water (6 mL) was added K2CO₃ (1.84 g, 13.29 mmol) at room temperature under nitrogen atmosphere and resulting mixture was degassed by bubbling nitrogen gas for 10 minutes. Subsequently, bis(1-diphenylphosphanylcyclopenta-2,4-dien-1-yl)iron dichloropalladium dichloromethane (542.64 mg, 664.49 mol) was added to the reaction mixture and stirred at 80° C. under nitrogen atmosphere for 16 hours. After completion of the reaction as indicated by liquid chromatography-mass spectrometry, the reaction mixture was allowed to cool to room temperature and poured into water (80 mL) and extracted with ethyl acetate (2×70 mL). Organic phases were combined and washed with saturated NaCl (80 mL), dried over anhydrous Na₂SO₄, filtered and concentrated under reduced pressure to get a crude residue which was purified by flash silica-gel (230-400 mesh) column using 0-100% ethyl acetate/hexane as an eluent to afford tert-butyl 2-(7-hydroxyquinoxalin-2-yl)-7-azaspiro[3.5]non-2-ene-7-carboxylate (5, 1.8 g, 4.80 mmol, 72% yield) as a pale yellow colored solid. UPLC-MS (ES+): m/z 368.2 [M+H]⁺.

### Step 4

Into a 250 mL single-neck round bottom flask containing a well-stirred solution of tert-butyl 2-(7-hydroxyquinoxalin-2-yl)-7-azaspiro[3.5]non-2-ene-7-carboxylate (5, 1.8 g, 4.90 mmol) in anhydrous 1,4-dioxane (45 mL) was purged with nitrogen for 5 minutes, added palladium hydroxide on carbon (20 wt. % loading, dry basis) (900 mg, 6.41 mmol) under nitrogen atmosphere and the resulting reaction suspension was stirred at room temperature under hydrogen bladder pressure for 3 hours. After completion of the reaction as indicated by liquid chromatography-mass spectrometry, the reaction mixture was filtered on a celite pad, and the pad was washed with ethyl acetate (500 mL). Filtrate was concentrated under reduced pressure to afford tert-butyl 2-(7-hydroxyquinoxalin-2-yl)-7-azaspiro[3.5]nonane-7-carboxylate (6, 1.7 g, 4.51 mmol, 92% yield) as a brown solid. UPLC-MS (ES+): m/z 370.2 [M+H]⁺.

### Step 5

Into a 100 mL single-neck round bottom flask containing a well-stirred solution of tert-butyl 2-(7-hydroxyquinoxalin-2-yl)-7-azaspiro[3.5]nonane-7-carboxylate (6, 1.7 g, 4.51 mmol) in anhydrous THF (45 mL) was added Cs₂CO₃ (4.41 g, 13.53 mmol) followed by 2,3,6-trifluorobenzonitrile (7, 6.76 mmol, 781.30 μL) at room temperature and the resulting suspension was stirred at room temperature under nitrogen atmosphere for 12 hours. After completion of the reaction, the reaction mixture was diluted with water (80 mL) and extracted with dichloromethane (2×70 mL). Combined organic phases were dried (anhydrous Na₂SO₄), filtered and the filtrate was concentrated under reduced pressure to get a crude residue, which was purified by flash silica-gel (230-400 mesh) column using 0-100% ethyl acetate/hexane as an eluent to afford tert-butyl 2-[7-(2-cyano-3,6-difluoro-phenoxy)quinoxalin-2-yl]-7-azaspiro[3.5]nonane-7-carboxylate (8, 1.6 g, 3.10 mmol, 69% yield) as a pale yellow solid. UPLC-MS (ES+): m/z 507.3 [M+H]⁺.

### Step 6

Into a 50 mL single-neck round bottom flask containing a well-stirred solution of tert-butyl 2-[7-(2-cyano-3,6-difluoro-phenoxy)quinoxalin-2-yl]-7-azaspiro[3.5]nonane-7-carboxylate (8, 800 mg, 1.58 mmol) and [methyl(sulfamoyl)amino]ethane (9, 436.49 mg, 3.16 mmol) in anhydrous N,N-dimethylformamide (20 mL) was added Cs₂CO₃ (1.29 g, 3.95 mmol) at ambient temperature and the resulting suspension was stirred at 65° C. for 24 hours. After completion of the reaction, the reaction mixture was diluted with water (50 mL) and extracted with ethyl acetate (2×80 mL). The combined organic phases were dried over anhydrous Na₂SO₄, filtered and the filtrate was concentrated under reduced pressure to get a crude residue which was purified by flash silica-gel (230-400 mesh) column using 0-100% ethyl acetate/hexane as an eluent to afford tert-butyl 2-[7-[2-cyano-3-[[ethyl(methyl)sulfamoyl]amino]-6-fluoro-phenoxy]quinoxalin-2-yl]-7-azaspiro[3.5]nonane-7-carboxylate (10, 400 mg, 614.67 mol, 39% yield) as a pale yellow solid. UPLC-MS (ES+): m/z 569.0 [M+H−^{t}Bu]⁺.

### Step 7

Into a 50 mL single-neck round bottom flask containing a well-stirred solution of tert-butyl 2-[7-[2-cyano-3-[[ethyl(methyl)sulfamoyl]amino]-6-fluoro-phenoxy]quinoxalin-2-yl]-7-azaspiro[3.5]nonane-7-carboxylate (10, 400 mg, 614.67 mol) in dichloromethane (4 mL) was added HCl, 4M in 1,4-dioxane, (4 M, 2 mL) was added at 0° C. The resulting mixture was stirred at ambient temperature for 2 hours. After completion of the reaction as indicated by liquid chromatography-mass spectrometry, the excess solvents were removed from the reaction mixture under reduced pressure to get a crude 2-(7-azaspiro[3.5]nonan-2-yl)-7-[2-cyano-3-[[ethyl(methyl)sulfamoyl]amino]-6-fluoro-phenoxy]quinoxaline (A-46, 340 mg, 593.87 mol, 97% yield, HCl salt) as a gummy solid. UPLC-MS (ES+): m/z 525.3 [M+H]⁺.

### Synthesis A47: Synthesis of N-[3-[3-(7-azaspiro[3.5]nonan-2-yl)quinoxalin-6-yl]oxy-2-cyano-4-fluoro-phenyl]propane-2-sulfonamide (A-47)

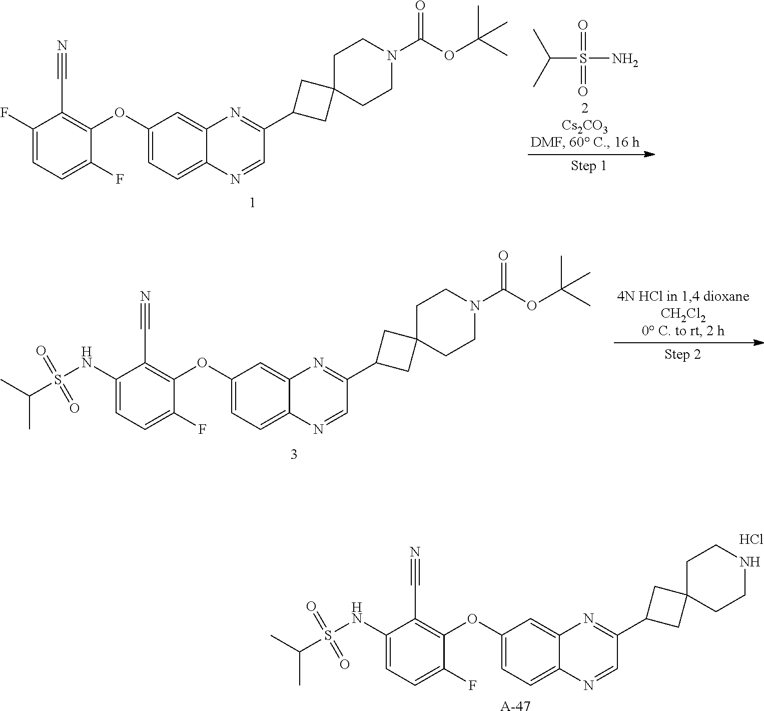

### Step 1

Into a 40 mL glass vial with pressure released cap containing a well-stirred solution of tert-butyl 2-[7-(2-cyano-3,6-difluoro-phenoxy)quinoxalin-2-yl]-7-azaspiro[3.5]nonane-7-carboxylate (1, 1 g, 1.95 mmol) and propane-2-sulfonamide (2, 481.47 mg, 3.91 mmol) were in N,N-dimethylformamide (10 mL) was added Cs₂CO₃ (1.59 g, 4.89 mmol) at room temperature. Reaction mixture was stirred at 60° C. for 16 hours. After completion of the reaction as indicated by TLC, reaction mixture was diluted with water (100 mL) and extracted with ethyl acetate (2×100 mL). Combined organic layer was dried over Na₂SO₄, filtered and concentrated under reduced pressure to get a crude residue, which was purified by flash silica-gel (230-400 mesh, 50 g) column using 0-100% ethyl acetate/hexane as an eluent to afford tert-butyl 2-[7-[2-cyano-6-fluoro-3-(isopropylsulfonylamino)phenoxy]quinoxalin-2-yl]-7-azaspiro[3.5]nonane-7-carboxylate (3, 1.1 g, 1.60 mmol, 82% yield) as a light brown gummy liquid. UPLC-MS(ES−): m/z 554.5 [M−^{t}Bu+H]⁺.

### Step 2

Into a 100 mL single-neck round-bottom flask containing a well stirred solution of tert-butyl 2-[7-[2-cyano-6-fluoro-3-(isopropylsulfonylamino)phenoxy]quinoxalin-2-yl]-7-azaspiro[3.5]nonane-7-carboxylate (3, 1.1 g, 1.61 mmol) in dichloromethane (15 mL) was added Hydrogen chloride, 4M in 1,4-dioxane (4 M, 401.42 L) at 0° C. Reaction mixture was stirred at room temperature for 2 hours. After completion of the starting material as indicated by TLC, solvents were concentrated under reduced pressure to afford N-[3-[3-(7-azaspiro[3.5]nonan-2-yl)quinoxalin-6-yl]oxy-2-cyano-4-fluoro-phenyl]propane-2-sulfonamide (A-47, 900 mg, 1.55 mmol, 97% yield, HCl salt) as an off-white solid. UPLC-MS (ES+): m/z 510.0 [M+H]⁺.

### Synthesis A48: Synthesis of (3R)-N-[3-[3-(7-azaspiro[3.5]nonan-2-yl)quinoxalin-6-yl]oxy-2-cyano-4-fluoro-phenyl]-3-fluoro-pyrrolidine-1-sulfonamide (A-48)

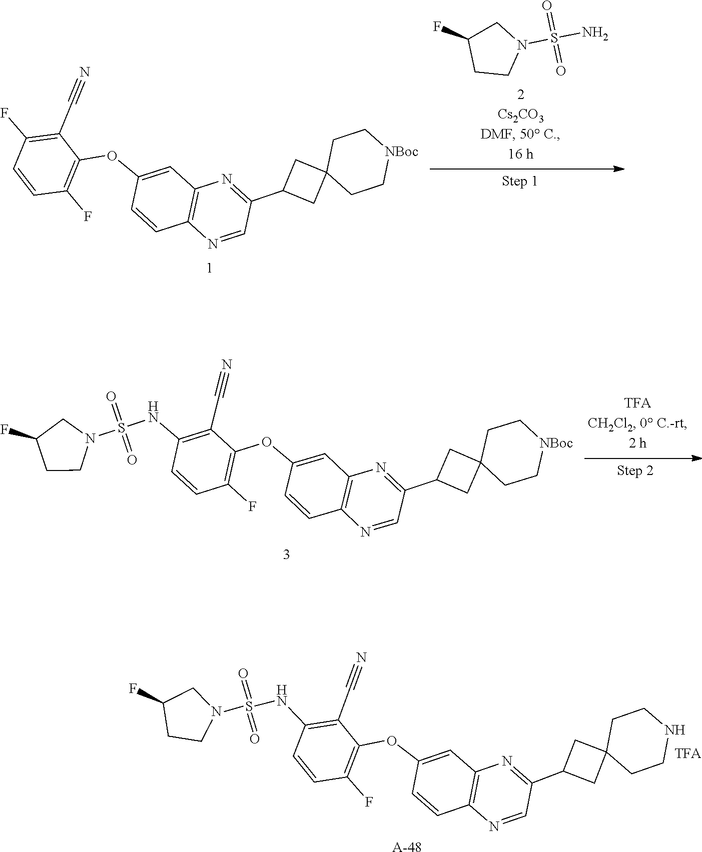 [SMILES: CC(C)(C)OC(=O)N1CCC2(CC1)CC(c1cnc3ccc(Oc4c(F)ccc(F)c4C#N)cc3n1)C2 ; NS(=O)(=O)N1CC[C@@H](F)C1 ; CC(C)(C)OC(=O)N1CCC2(CC1)CC(c1cnc3ccc(Oc4c(F)ccc(NS(=O)(=O)N5CC[C@@H](F)C5)c4C#N)cc3n1)C2 ; N#Cc1c(NS(=O)(=O)N2CC[C@@H](F)C2)ccc(F)c1Oc1ccc2ncc(C3CC4(CCNCC4)C3)nc2c1 ; "A-48"]

### Step 1

Into a 10 mL reaction vial containing a stirred solution of tert-butyl 2-[7-(2-cyano-3,6-difluoro-phenoxy)quinoxalin-2-yl]-7-azaspiro[3.5]nonane-7-carboxylate (1, 100 mg, 197.42 mol) in N,N-dimethylformamide (2 mL) were added (3R)-3-fluoropyrrolidine-1-sulfonamide (2, 39.84 mg, 236.90 mol) and Cs₂CO₃ (192.97 mg, 592.25 mol) at room temperature. The reaction was stirred at 50° C. for 16 hours. After completion of the reaction, the reaction mixture was cooled, diluted with ice water (20 mL) and extracted with ethyl acetate (2×20 mL). The organic layer was dried over anhydrous Na₂SO₄, filtered and concentrated under reduced pressure to afford tert-butyl 2-[7-[2-cyano-6-fluoro-3-[[(3R)-3-fluoropyrrolidin-1-yl]sulfonylamino]phenoxy]quinoxalin-2-yl]-7-azaspiro[3.5]nonane-7-carboxylate (3, 150 mg, 142.04 mol, 72% yield) as a brown gummy liquid. UPLC-MS (ES+): m/z 599.0 [M−^{t}Bu+H]⁺.

### Step 2

Into a 25 mL single neck round bottom flask containing a well stirred solution of tert-butyl 2-[7-[2-cyano-6-fluoro-3-[[(3R)-3-fluoropyrrolidin-1-yl]sulfonylamino]phenoxy]quinoxalin-2-yl]-7-azaspiro[3.5]nonane-7-carboxylate (3, 150 mg, 229.10 mol) in dichloromethane (2 mL) was added trifluoroacetic acid (2.29 mmol, 175.32 L) at 0° C. The reaction was stirred at room temperature for 2 hours. The reaction mixture was concentrated under reduced pressure and triturated with methyl tert-butyl ether (10 mL) to afford (3R)-N-[3-[3-(7-azaspiro[3.5]nonan-2-yl)quinoxalin-6-yl]oxy-2-cyano-4-fluoro-phenyl]-3-fluoro-pyrrolidine-1-sulfonamide (A-48, 180 mg, 220.75 mol, 96% yield, TFA salt) as a brown gummy liquid. UPLC-MS (ES+): m/z 555.4 [M+H]⁺.

### Synthesis A49: Synthesis of N-[3-[3-(7-azaspiro[3.5]nonan-2-yl)quinoxalin-6-yl]oxy-2-cyano-4-fluoro-phenyl]cyclopentanesulfonamide (A-49)

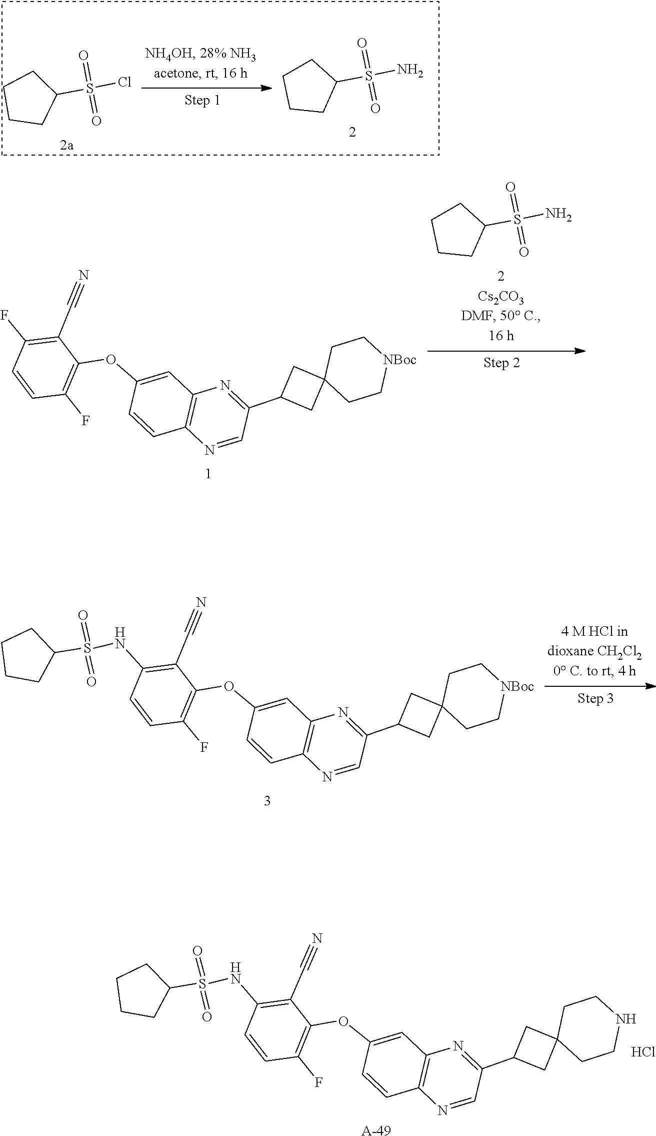

### Step 1

Into a 250 mL two-neck round-bottom flask containing a well stirred solution of cyclopentanesulfonyl chloride (2a, 5 g, 29.65 mmol, 3.77 mL) in acetone (25 mL) was added ammonium hydroxide, 28% NH₃ (1.01 mol, 39.26 mL) at 0° C. and resulting reaction mixture was stirred at room temperature for 16 hours. After completion of the reaction, the reaction mixture was quenched with 1.5 M HCl at 0° C. and extracted with dichloromethane (2×100 mL). Combined organic layer was dried over anhydrous Na₂SO₄, filtered and concentrated under reduced pressure to afford cyclopentanesulfonamide (2, 3 g, 18.30 mmol, 62% yield) as a brown liquid. UPLC-MS (ES−): m/z 148.2 [M−H]⁻.

### Step 2

Into a 40 mL pressure vial containing a well stirred solution of tert-butyl 2-[7-(2-cyano-3,6-difluoro-phenoxy)quinoxalin-2-yl]-7-azaspiro[3.5]nonane-7-carboxylate (1, 0.350 g, 690.96 mol) and cyclopentanesulfonamide (2, 0.154 g, 1.04 mmol) in N,N-dimethylformamide (10 mL) was added Cs₂CO₃ (787.95 mg, 2.42 mmol) at room temperature under nitrogen atmosphere. Resulting reaction mixture was stirred at 50° C. for 16 hours. After completion of the reaction, the reaction mixture was diluted with water (40 mL) and extracted with ethyl acetate (3×30 mL). Combined organic layer was washed with water (30 mL), dried over anhydrous Na₂SO₄, filtered and concentrated under reduced pressure to get a crude residue which was purified by the flash (25 g, 230-400 silica-gel mesh) column chromatography using 0-100% ethyl acetate in hexane as an eluent while desired product was eluting at 40-70% of mobile phase to afford tert-butyl 2-[7-[2-cyano-3-(cyclopentylsulfonylamino)-6-fluoro-phenoxy]quinoxalin-2-yl]-7-azaspiro[3.5]nonane-7-carboxylate (3, 0.320 g, 392.61 mol, 57% yield) as a brown gummy liquid. UPLC-MS (ES−): m/z 634.3 [M−H]⁻.

### Step 3

Into a 100 mL single-neck round-bottom flask containing a well stirred solution of tert-butyl 2-[7-[2-cyano-3-(cyclopentylsulfonylamino)-6-fluoro-phenoxy]quinoxalin-2-yl]-7-azaspiro[3.5]nonane-7-carboxylate (3, 0.320 g, 503.34 mol) in anhydrous dichloromethane (10 mL) was added 4 M HCl in dioxane (503.34 mol, 1.0 mL) at 0° C. under nitrogen atmosphere. Resulting reaction mixture was stirred at room temperature for 4 hours. After completion of the reaction, the reaction mixture was concentrated under reduced pressure to afford N-[3-[3-(7-azaspiro[3.5]nonan-2-yl)quinoxalin-6-yl]oxy-2-cyano-4-fluoro-phenyl]cyclopentanesulfonamide (A-49, 0.320 g, 441.89 mol, 88% yield, HCl salt) as an off-white solid. UPLC-MS (ES+): m/z 536.2 [M+H]⁺.

### Synthesis A50: Synthesis of 2-(7-azaspiro[3.5]nonan-2-yl)-7-[2-cyano-6-fluoro-3-[[2-hydroxyethyl(methyl)sulfamoyl]amino]phenoxy]quinoxaline (A-50)

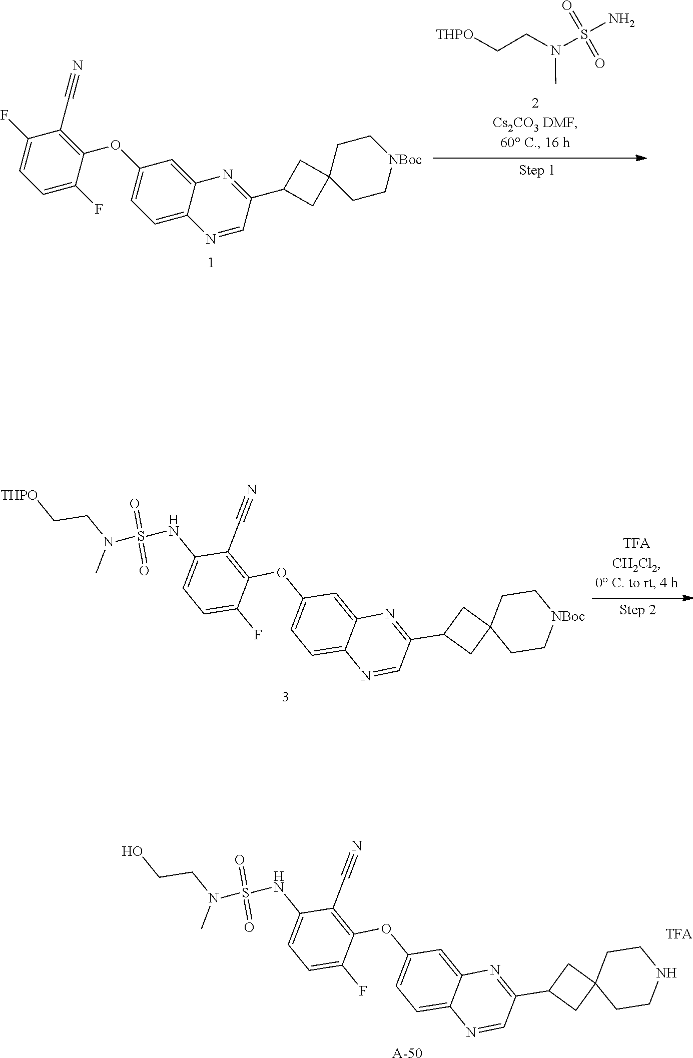

### Step 1

Into a 20 mL glass vial containing a well-stirred solution of tert-butyl 2-[7-(2-cyano-3,6-difluoro-phenoxy)quinoxalin-2-yl]-7-azaspiro[3.5]nonane-7-carboxylate (1, 500 mg, 987.08 mol) and 2-[2-[methyl(sulfamoyl)amino]ethoxy]tetrahydropyran (2, 470.45 mg, 1.97 mmol) in N,N-dimethylformamide (5 mL) was added Cs₂CO₃ (804.03 mg, 2.47 mmol) at room temperature and the resulting suspension was stirred at 60° C. for 16 hours. After completion of the reaction, the reaction mixture was diluted with water (50 mL) and extracted with ethyl acetate (2×80 mL). Combined organic phases were dried over anhydrous Na₂SO₄, filtered and the filtrate was concentrated under reduced pressure to get a crude residue, which was purified by flash silica-gel (230-400 mesh) column with 0-100% ethyl acetate/hexane to afford tert-butyl 2-[7-[2-cyano-6-fluoro-3-[[methyl(2-tetrahydropyran-2-yloxyethyl)sulfamoyl]amino]phenoxy]quinoxalin-2-yl]-7-azaspiro[3.5]nonane-7-carboxylate (3, 270 mg, 298.00 mol, 30% yield) as a pale yellow solid. UPLC-MS (ES−): m/z 723.4 [M−H]⁻.

### Step 2

Into a 50 mL single neck round-bottom flask containing a well-stirred solution of tert-butyl 2-[7-[2-cyano-6-fluoro-3-[[methyl(2-tetrahydropyran-2-yloxyethyl)sulfamoyl]amino]phenoxy]quinoxalin-2-yl]-7-azaspiro[3.5]nonane-7-carboxylate (3, 270 mg, 298.00 mol) in anhydrous dichloromethane (3 mL) was added trifluoroacetic acid (32.67 mmol, 2.5 mL) at 0° C. under nitrogen atmosphere and stirred at room temperature for 4 hours. After completion of the reaction, the reaction mixture was concentrated under reduced pressure to get the crude which was co-distilled with hexane (2×3 mL) followed by triturated with methyl tert-butyl ether (2×3 mL), decanted and dried under reduced pressure to afford 2-(7-azaspiro[3.5]nonan-2-yl)-7-[2-cyano-6-fluoro-3-[[2-hydroxyethyl(methyl)sulfamoyl]-amino]phenoxy]quinoxaline (A-50, 210 mg, 218.14 mol, 73% yield, TFA salt) as a brown thick liquid. UPLC-MS(ES+): m/z 541.3 [M+H]⁺.

### Synthesis A51: Synthesis of (3S)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-51)

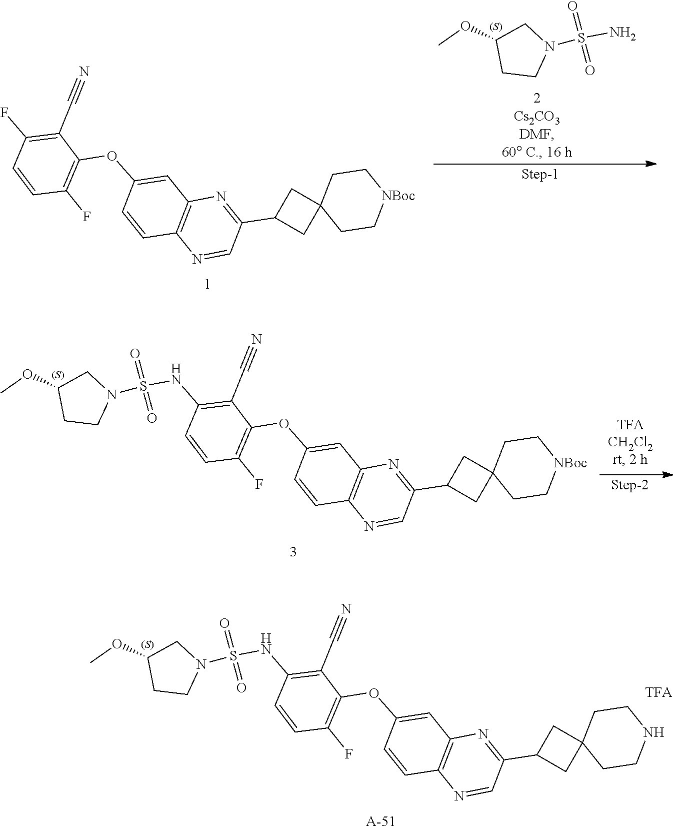

### Step 1

Into a 20 mL pressure vial containing a well-stirred solution of tert-butyl 2-[7-(2-cyano-3,6-difluoro-phenoxy)quinoxalin-2-yl]-7-azaspiro[3.5]nonane-7-carboxylate (1, 500 mg, 987.08 mol) and (3S)-3-methoxypyrrolidine-1-sulfonamide (2, 266.85 mg, 1.48 mmol) in N,N-dimethylformamide (5 mL) was added Cs₂CO₃ (964.83 mg, 2.96 mmol) at room temperature and stirred at 60° C. for 16 hours. After completion of the reaction, the reaction mixture was diluted with water (20 mL) and extracted with ethyl acetate (2×70 mL). Combined organic layers were washed with saturated NaCl (50 mL), dried over anhydrous Na₂SO₄, concentrated under reduced pressure to get crude residue which was purified by flash (230-400 silica mesh) column chromatography using 0-100% ethyl acetate/hexane as mobile phase to afford tert-butyl 2-[7-[2-cyano-6-fluoro-3-[[(3S)-3-methoxypyrrolidin-1-yl]sulfonylamino]phenoxy]quinoxalin-2-yl]-7-azaspiro[3.5]nonane-7-carboxylate (3, 450 mg, 492.68 mol, 50% yield) as a brown gummy liquid. UPLC-MS (ES+): m/z 667.2 [M+H]⁺.

### Step 2

Into a 50 mL single-neck round-bottom flask containing a well-stirred solution of tert-butyl 2-[7-[2-cyano-6-fluoro-3-[[(3S)-3-methoxypyrrolidin-1-yl]sulfonylamino]phenoxy]quinoxalin-2-yl]-7-azaspiro[3.5]nonane-7-carboxylate (3, 450 mg, 674.90 mol) in anhydrous dichloromethane (6 mL) was added trifluoroacetic acid (6.75 mmol, 516.48 μL) at 0° C. Resulting reaction mixture was stirred at room temperature for 2 hours. After completion of the reaction, the reaction mixture was concentrated under reduced pressure to get a crude residue which was triturated with methyl tert-butyl ether (20 mL) and dried under reduced pressure to afford (3S)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-51, 450 mg, 575.17 mol, 85% yield, TFA salt) as a brown semi solid. UPLC-MS (ES+): m/z 567.2 [M+H]⁺.

### Synthesis A52: Synthesis of (3R)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-52)

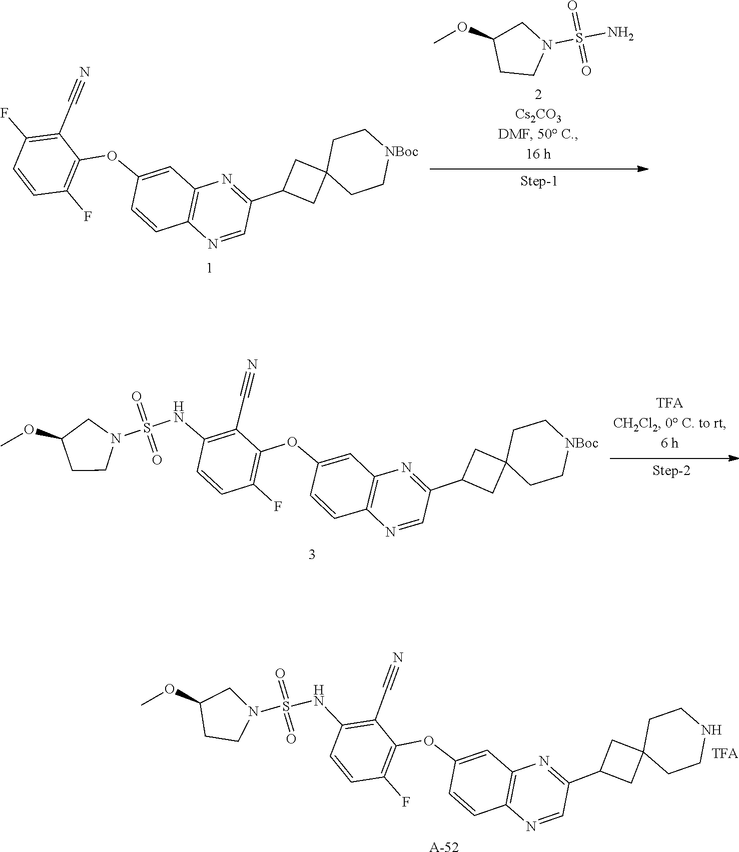 [SMILES: CC(C)(C)OC(=O)N1CCC2(CC1)CC(c1cnc3ccc(Oc4c(F)ccc(F)c4C#N)cc3n1)C2 ; CO[C@@H]1CCN(S(N)(=O)=O)C1 ; CO[C@@H]1CCN(S(=O)(=O)Nc2ccc(F)c(Oc3ccc4ncc(C5CC6(CCN(C(=O)OC(C)(C)C)CC6)C5)nc4c3)c2C#N)C1 ; CO[C@@H]1CCN(S(=O)(=O)Nc2ccc(F)c(Oc3ccc4ncc(C5CC6(CCNCC6)C5)nc4c3)c2C#N)C1 ; "A-52"]

### Step 1

Into a 20 mL glass vial with pressure released cap containing a well-stirred solution of tert-butyl 2-[7-(2-cyano-3,6-difluoro-phenoxy)quinoxalin-2-yl]-7-azaspiro[3.5]nonane-7-carboxylate (1, 250 mg, 458.99 mol) and (3R)-3-methoxypyrrolidine-1-sulfonamide (2, 107.54 mg, 596.69 mol) in N,N-dimethylformamide (5 mL) was added Cs₂CO₃ (448.65 mg, 1.38 mmol) at room temperature. Resulting reaction was stirred at 50° C. for 16 hours. The reaction mixture was diluted with water (50 mL) and stirred for 10 minutes, resulting solid impurity was avoided by filtration sintered funnel, filtrate was extracted with ethyl acetate (2×50 mL), Combined organic layers were dried over anhydrous Na₂SO₄, filtered and concentrated under reduced pressure to afford tert-butyl 2-[7-[2-cyano-6-fluoro-3-[[(3R)-3-methoxypyrrolidin-1-yl]sulfonylamino]phenoxy]quinoxalin-2-yl]-7-azaspiro[3.5]nonane-7-carboxylate (3, 300 mg, 427.03 mol, 93% yield) as a light brown gummy liquid. UPLC-MS (ES−): m/z 665.3 [M−H]⁻.

### Step 2

Into a 50 mL single-neck round-bottom flask containing a well-stirred solution of tert-butyl 2-[7-[2-cyano-6-fluoro-3-[[(3R)-3-methoxypyrrolidin-1-yl]sulfonylamino]phenoxy]quinoxalin-2-yl]-7-azaspiro[3.5]nonane-7-carboxylate (3, 300 mg, 427.44 mol) in dichloromethane (10 mL) was added trifluoroacetic acid (4.27 mmol, 327.09 L) at 0° C. Resulting reaction mixture was stirred at room temperature for 6 hours. After completion of the starting material, reaction mixture was concentrated directly under reduced pressure to get crude residue, which was triturated with methyl tert-butyl ether (2×20 mL) and dried under reduced pressure to afford (3R)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-52, 230 mg, 314.45 mol, 74% yield, TFA salt) as a brown semi solid. UPLC-MS (ES+): m/z 567.4 [M+H]⁺.

### Synthesis A53: Synthesis of 3-(7-azaspiro[3.5]nonan-2-yl)-6-[3-[[ethyl(methyl)sulfamoyl]amino]-2,6-difluoro-phenoxy]-4-oxo-quinazoline (A-53)

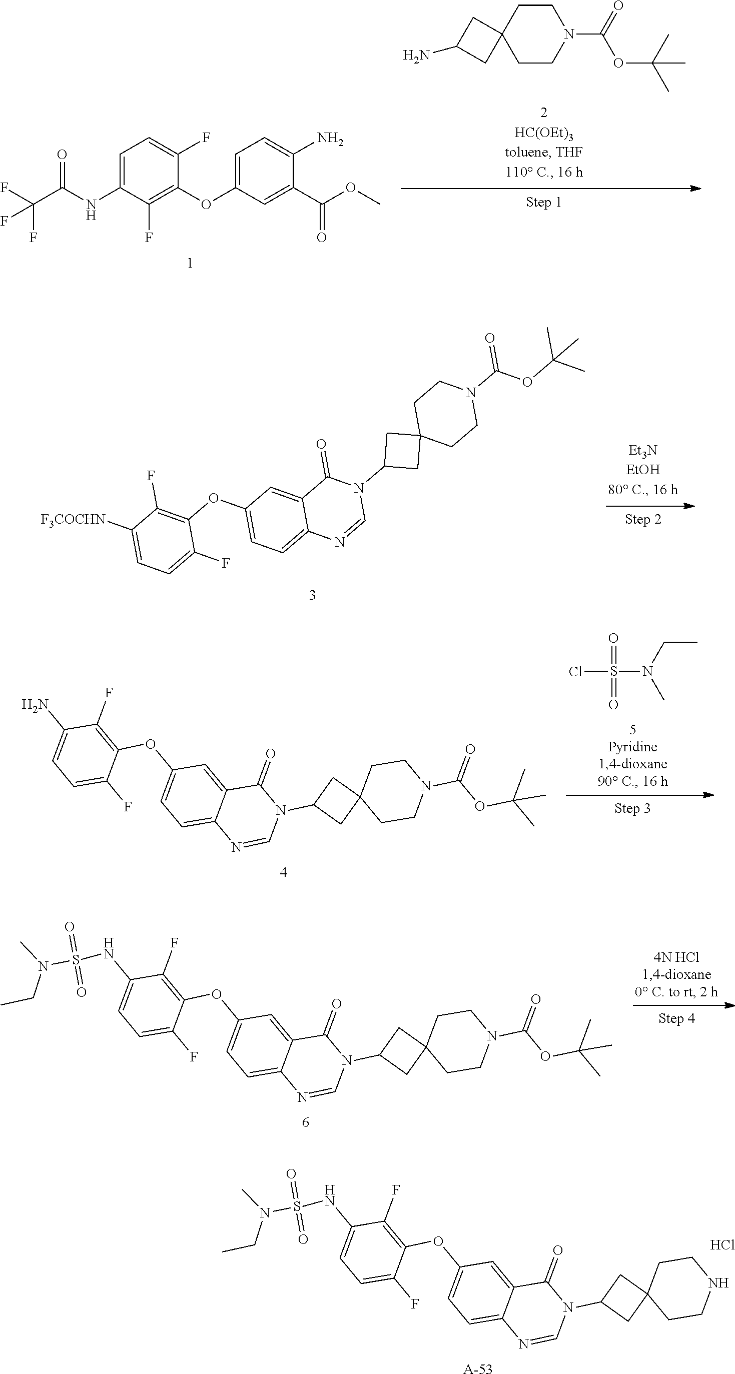

### Step 1

Into a 250 mL single-neck round-bottom flask containing a well stirred solution of methyl 2-amino-5-[2,6-difluoro-3-[(2,2,2-trifluoroacetyl)amino]phenoxy]benzoate (1, 5.0 g, 12.81 mmol; see intermediate 7 in the synthesis of A55) and tert-butyl 2-amino-7-azaspiro[3.5]nonane-7-carboxylate (2, 3.39 g, 14.09 mmol) in anhydrous THF (20 mL) and toluene (80 mL) was added triethyl orthoformate (25.62 mmol, 4.26 mL) at room temperature under nitrogen atmosphere and stirred at 110° C. for 16 hours. After completion of the reaction, the reaction mixture was allowed to cool to room temperature and poured into cold water (50 mL). The layers were separated, and the aqueous layer was extracted with ethyl acetate (2×70 mL). Combined organic layers were washed with saturated NaCl (50 mL), dried over anhydrous Na₂SO₄, filtered and concentrated under reduced pressure to afford tert-butyl 2-[6-[2,6-difluoro-3-[(2,2,2-trifluoroacetyl)amino]phenoxy]-4-oxo-quinazolin-3-yl]-7-azaspiro[3.5]nonane-7-carboxylate (3, 4.0 g, 2.56 mmol, 20% yield) as an off-white solid. UPLC-MS (ES+): m/z 609.2 [M+H]⁺.

### Step 2

Into a 250 mL single-neck round-bottom flask containing a well-stirred solution of tert-butyl 2-[6-[2,6-difluoro-3-[(2,2,2-trifluoroacetyl)amino]phenoxy]-4-oxo-quinazolin-3-yl]-7-azaspiro[3.5]nonane-7-carboxylate (3, 4.0 g, 2.56 mmol) in ethanol (40 mL) was added triethylamine (25.63 mmol, 3.57 mL) at room temperature under nitrogen atmosphere and stirred at 80° C. for 16 hours. Stirring was continued at 80° C. for additional 32 hours. After completion of the reaction, the reaction mixture was allowed to cool to room temperature and concentrated under reduced pressure to afford crude residue. Crude was diluted with water (50 mL), ethyl acetate (100 mL) and aqueous layer was extracted with ethyl acetate (2×50 mL). Combined organic layers were washed with saturated NaCl (50 mL), dried over anhydrous Na₂SO₄, filtered and filtrate was concentrated under reduced pressure to get a crude residue which was purified by flash silica-gel (230-400 mesh) column using 0-100% ethyl acetate/hexane as an eluent to afford tert-butyl 2-[6-(3-amino-2,6-difluoro-phenoxy)-4-oxo-quinazolin-3-yl]-7-azaspiro[3.5]nonane-7-carboxylate (4, 1.5 g, 1.34 mmol, 52% yield) as an off-white solid. UPLC-MS (ES+): m/z 513.2 [M+H]⁺. The isolated product showed 52% purity by UPLC-MS and was taken to the next step.

### Step 3

Into a 250 mL single-neck round-bottom flask containing a well-stirred solution of tert-butyl 2-[6-(3-amino-2,6-difluoro-phenoxy)-4-oxo-quinazolin-3-yl]-7-azaspiro[3.5]nonane-7-carboxylate (4, 1.4 g, 1.25 mmol) in 1,4-dioxane (20 mL) were added pyridine (12.51 mmol, 1.01 mL) and N-ethyl-N-methyl-sulfamoyl chloride (5, 6.26 mmol, 771.27 L) at 0° C. Resulting reaction mixture was stirred at 90° C. for 16 hours. After completion of the reaction, the reaction mixture allowed to cool to room temperature and diluted with water (50 mL) and ethyl acetate (70 mL). The layers were separated, and aqueous layer was extracted with ethyl acetate (2×30 mL). Combined organic layers were washed with saturated NaCl (50 mL), dried over anhydrous Na₂SO₄, filtered and concentrated under reduced pressure to get a crude residue which was purified by flash silica-gel (230-400 mesh) column using 0-100% ethyl acetate/hexane as an eluent to afford tert-butyl 2-[6-[3-[[ethyl(methyl)sulfamoyl]amino]-2,6-difluoro-phenoxy]-4-oxo-quinazolin-3-yl]-7-azaspiro[3.5]nonane-7-carboxylate (6, 250 mg, 311.66 mol, 25% yield) as an off-white solid. UPLC-MS (ES+): m/z 634.2 [M+H]⁺.

### Step 4

Into 25 mL single-neck round-bottom flask containing a well-stirred solution of tert-butyl 2-[6-[3-[[ethyl(methyl)sulfamoyl]amino]-2,6-difluoro-phenoxy]-4-oxo-quinazolin-3-yl]-7-azaspiro[3.5]nonane-7-carboxylate (6, 250 mg, 311.66 mol) in dichloromethane (3 mL) was added HCl in 1,4-dioxane (4.0 M, 2.00 mL) dropwise at 0° C. under nitrogen atmosphere and reaction mixture was stirred at room temperature for 2 hours. After completion of the reaction, the reaction mixture was concentrated under reduced pressure to get a crude which was co-distilled with hexane (2×10 mL). Obtained crude material was triturated with methyl tert-butyl ether (2×5 mL) and dried under reduced pressure to afford 3-(7-azaspiro[3.5]nonan-2-yl)-6-[3-[[ethyl(methyl)sulfamoyl]amino]-2,6-difluoro-phenoxy]-4-oxo-quinazoline (A-53, 210 mg, 305.76 mol, 98% yield, HCl salt) as an off-white solid. UPLC-MS (ES+): m/z 534.2 [M+H]⁺.

### Synthesis A54: Synthesis of 3-(7-azaspiro[3.5]nonan-2-yl)-6-[2,6-difluoro-3-[[2-hydroxyethyl(methyl)sulfamoyl]amino]phenoxy]-4-oxo-quinazoline (A-54)

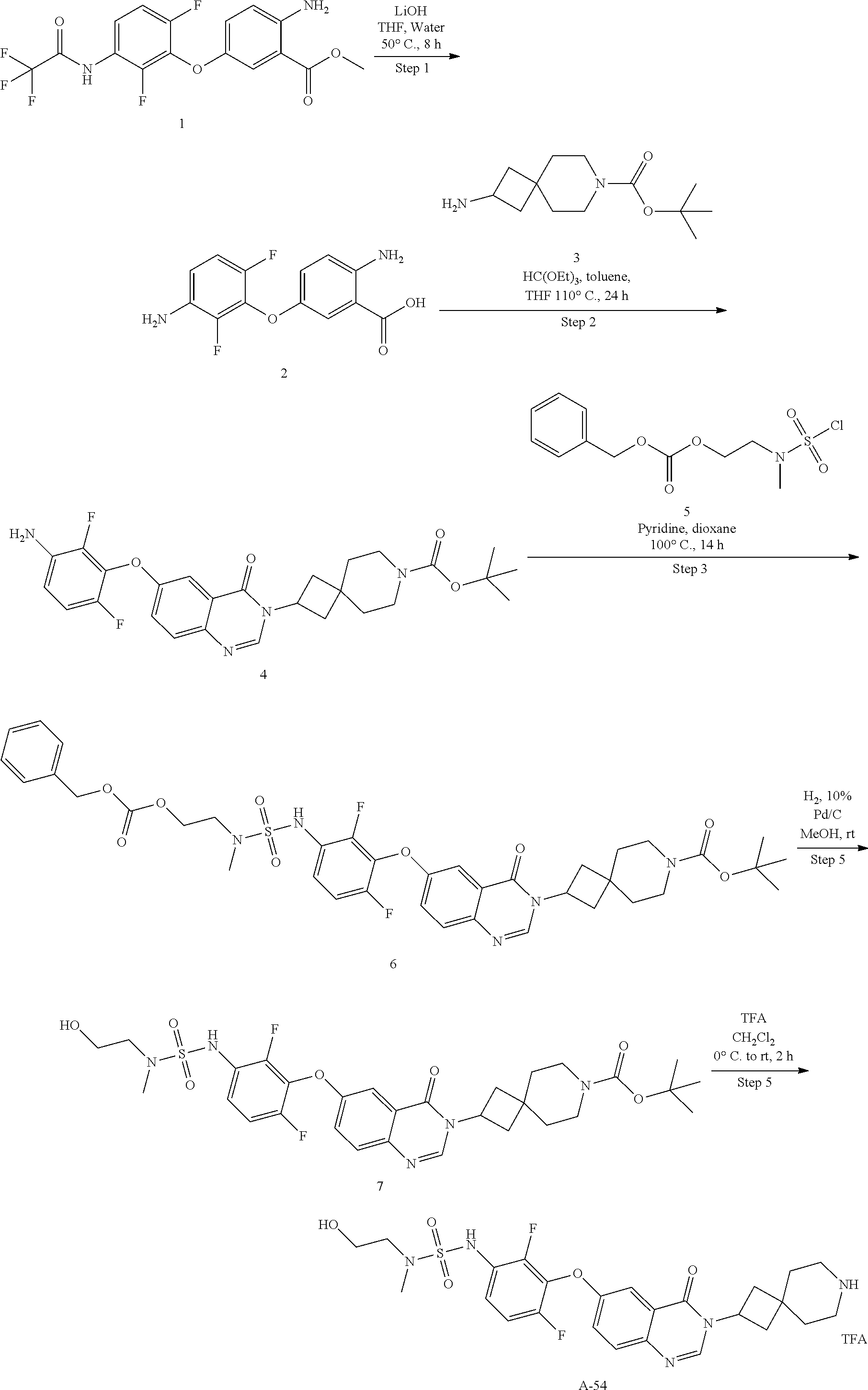

### Step 1

Into a 500 mL single neck round bottom flask containing a well stirred solution of methyl 2-amino-5-[2,6-difluoro-3-[(2,2,2-trifluoroacetyl)amino]phenoxy]benzoate (1, 9 g, 22.83 mmol; see intermediate 7 in the synthesis of A55) in THF (70 mL) and water (30 mL) was added lithium hydroxide monohydrate (1.92 g, 45.66 mmol) at 0-5° C. The resulted mixture was stirred at 50° C. for 8 hours. After completion of the reaction, solvent was removed under reduced pressure to get crude residue which was diluted with water and neutralized with 1.5 N HCl, a solid precipitate formed. Obtained precipitate was filtered and dried under reduced pressure to get 2-amino-5-(3-amino-2,6-difluoro-phenoxy)benzoic acid (2, 6 g, 20.34 mmol, 89% yield) as a grey-colored solid. UPLC-MS (ES+): m/z 281.0 [M+H]⁺.

### Step 2

Into a 250 mL sealed tube containing a stirred solution of 2-amino-5-(3-amino-2,6-difluoro-phenoxy)benzoic acid (2, 4 g, 13.56 mmol) in anhydrous Toluene (30 mL) was added tert-butyl 2-amino-7-azaspiro[3.5]nonane-7-carboxylate (3, 4.89 g, 20.34 mmol) and Triethyl orthoformate (27.12 mmol, 4.51 mL) in portions at room temperature under nitrogen atmosphere. After complete addition, the reaction mixture was stirred at 110° C. for 24 h under nitrogen atmosphere. After completion of the reaction, the reaction mixture was diluted with ethyl acetate (200 mL) and washed successively with H₂O (2×100 mL) and saturated NaCl (2×100 mL). The separated organic layer was dried (anhydrous Na₂SO₄), filtered and evaporated under reduced pressure to get a crude residue which was purified by silica gel column chromatography with 0-100% ethyl acetate/hexane as gradient while desired product eluted at 55-65% to afford tert-butyl 2-[6-(3-amino-2,6-difluoro-phenoxy)-4-oxo-quinazolin-3-yl]-7-azaspiro[3.5]nonane-7-carboxylate (4, 5 g, 8.97 mmol, 66% yield) as an off-white solid. UPLC-MS (ES+): m/z 513.2 [M+H]⁺.

### Step 3

Into a 100 mL sealed tube containing a well stirred solution of tert-butyl 2-[6-(3-amino-2,6-difluoro-phenoxy)-4-oxo-quinazolin-3-yl]-7-azaspiro[3.5]nonane-7-carboxylate (4, 1 g, 1.79 mmol) in 1,4 Dioxane (30.00 mL) was added Pyridine (10.77 mmol, 867.50 L) followed by the addition of benzyl 2-[chlorosulfonyl(methyl)amino]ethyl carbonate (5, 2.63 g, 7.18 mmol) at room temperature under nitrogen atmosphere and stirred at 100° C. for 14 hours. After completion of the reaction, the reaction mixture was concentrated directly under reduced pressure to get a crude residue which was purified by silica gel column chromatography with 0-100% ethyl acetate/hexane as gradient while desired product eluted at 55-60% to afford tert-butyl 2-[6-[3-[[2-benzyloxycarbonyloxyethyl(methyl)sulfamoyl]amino]-2,6-difluoro-phenoxy]-4-oxo-quinazolin-3-yl]-7-azaspiro[3.5]nonane-7-carboxylate (6, 360 mg, 408.76 mol, 23% yield) as an off-white solid. UPLC-MS (ES+): m/z 784.3 [M+H]⁺.

### Step 4

Into a 25 mL single-neck round-bottom flask containing a stirred solution of tert-butyl 2-[6-[3-[[2-benzyloxycarbonyloxyethyl(methyl)sulfamoyl]amino]-2,6-difluoro-phenoxy]-4-oxo-quinazolin-3-yl]-7-azaspiro[3.5]nonane-7-carboxylate (6, 360 mg, 372.02 mol) in methanol (10 mL). Reaction mixture was purged with nitrogen for five minutes followed by addition of Pd/C (10% on carbon (wetted with ca. 55% water)) (100 mg, 939.67 mol) and stirred under hydrogen bladder pressure at room temperature for 16 hours. After completion of the reaction, the reaction mixture was filtered through a celite bed and the filtrate was concentrated under reduced pressure to afford crude tert-butyl 2-[6-[2,6-difluoro-3-[[2-hydroxyethyl(methyl)sulfamoyl]amino]phenoxy]-4-oxo-quinazolin-3-yl]-7-azaspiro[3.5]nonane-7-carboxylate (7, 240 mg, 310.29 mol, 83% yield) as a colorless oil. UPLC-MS (ES+): m/z 650.3 [M+H]⁺.

### Step 5

Into a 50 mL double neck round-bottom flask containing a well stirred solution of tert-butyl 2-[6-[2,6-difluoro-3-[[2-hydroxyethyl(methyl)sulfamoyl]amino]phenoxy]-4-oxo-quinazolin-3-yl]-7-azaspiro[3.5]nonane-7-carboxylate (7, 240 mg, 369.40 mol) in dichloromethane (5.27 mL) was added trifluoroacetic acid (7.39 mmol, 565.37 L) at 0° C. under nitrogen atmosphere and stirred at room temperature for 2 hours. After completion of the reaction, the reaction mixture was concentrated under reduced pressure to get a crude residue which was triturated with methyl tert-butyl ether (2×5 mL) and dried under reduced pressure to afford crude 3-(7-azaspiro[3.5]nonan-2-yl)-6-[2,6-difluoro-3-[[2-hydroxyethyl(methyl)sulfamoyl]amino]phenoxy]-4-oxo-quinazoline (A-54, 250 mg, 308.92 mol, 84% yield, TFA salt) as a brown solid. UPLC-MS (ES+): m/z 550.2 [M+H]⁺.

### Synthesis A55: Synthesis of 3-[6-[3-[[ethyl(methyl)sulfamoyl]amino]1-2,6-difluoro-phenoxy]-4-oxo-quinazolin-3-yl]-1-oxa-8-azaspiro[4.5]decane (A-55)

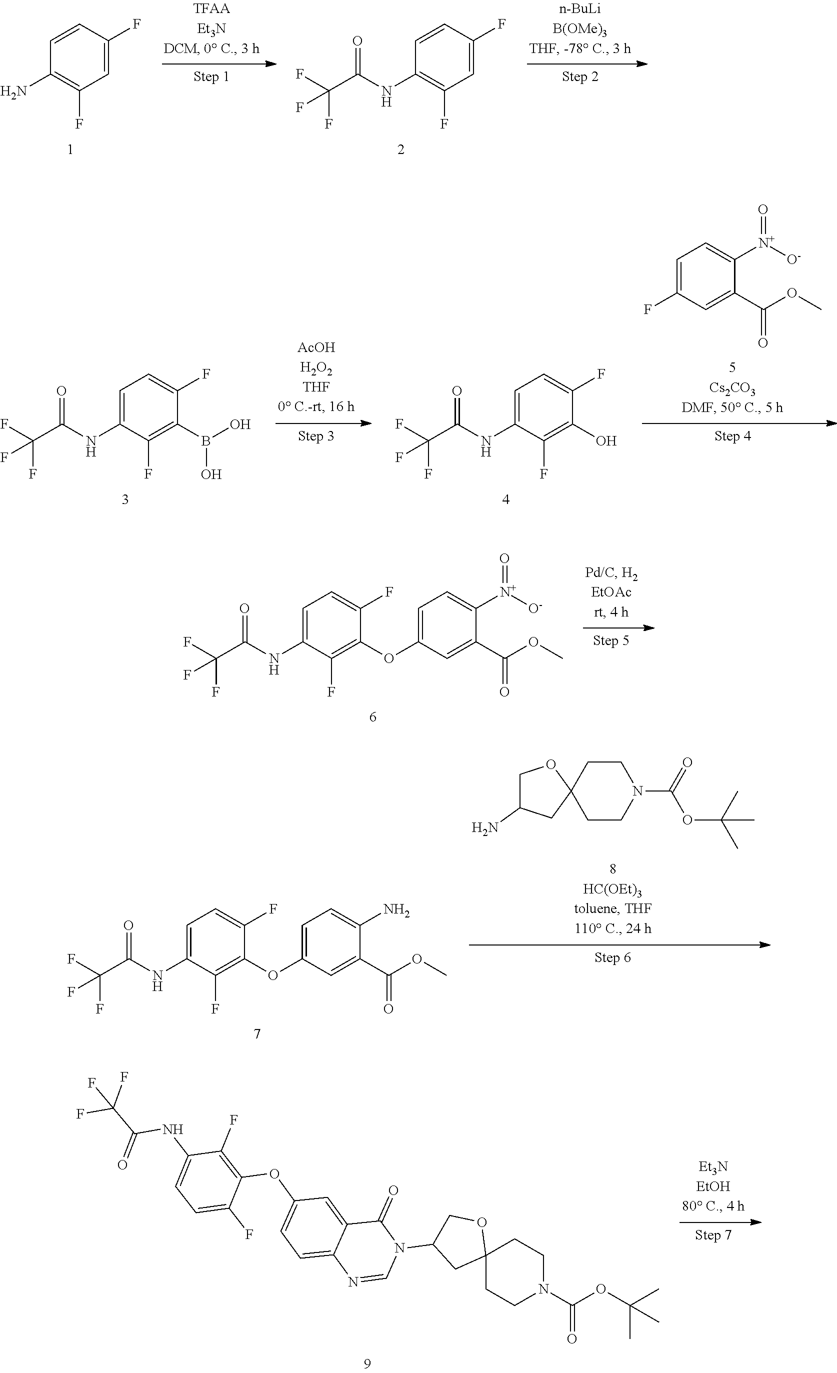

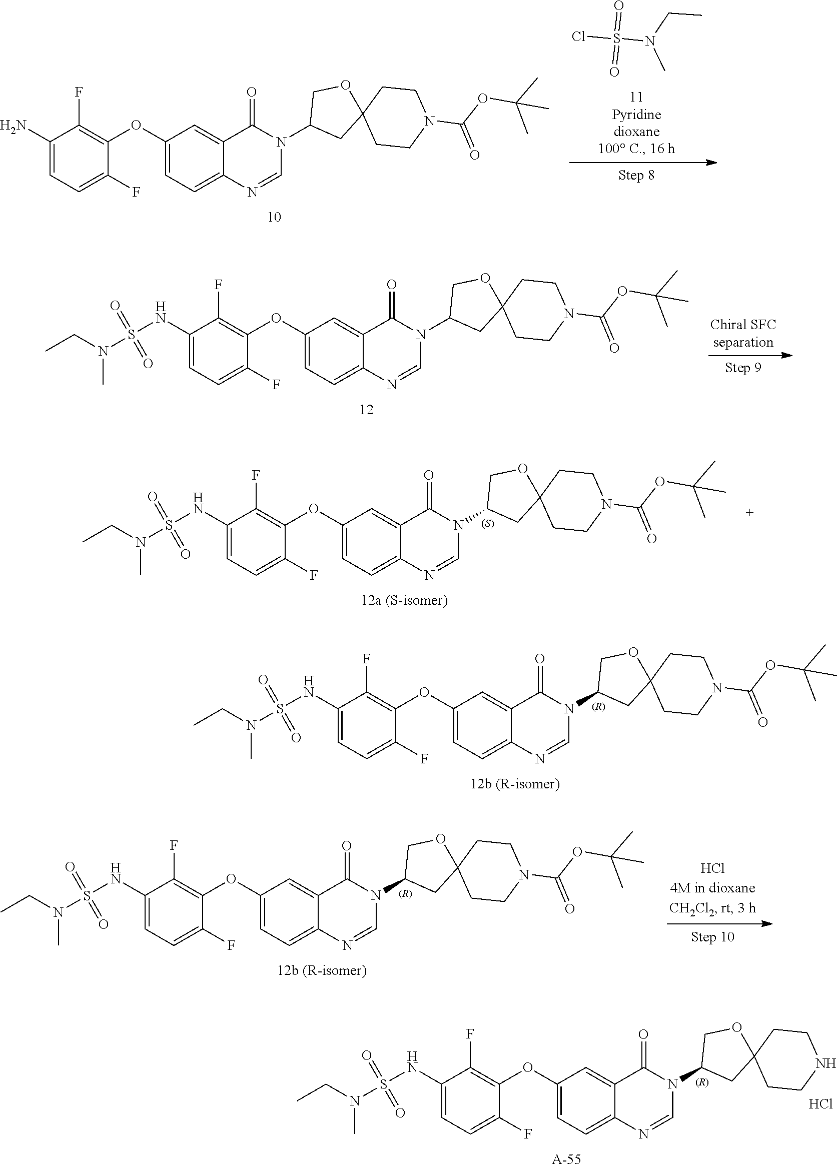

### Step 1

Into a two-neck 250 mL round-bottom flask containing a well stirred solution of 2,4-difluoroaniline (1, 387.28 mmol, 39.43 mL) in dichloromethane (500 mL) were added triethylamine (1, 1.16 mol, 161.93 mL) and trifluoroacetic anhydride (580.91 mmol, 80.75 mL) under nitrogen atmosphere at 0° C. The reaction mixture was stirred at 0° C. for 3 hours. After completion of the reaction, the reaction mixture was quenched with water and extracted with dichloromethane. The combined organic layers were washed with saturated NaCl, dried over Na₂SO₄, filtered and concentrated under reduced pressure to get a crude residue. The crude residue was purified by flash silica gel (230-400 mesh) column using 0-30% ethyl acetate/petroleum ether as an eluent to afford N-(2,4-difluorophenyl)-2,2,2-trifluoro-acetamide (2, 80 g, 273.64 mmol, 71% yield) as yellow needle shaped crystal. UPLC-MS (ES−): m/z 224.1 [M−H]⁻.

### Step 2

Into a 1000 mL three-neck round-bottom flask containing a well stirred solution of N-(2,4-difluorophenyl)-2,2,2-trifluoro-acetamide (2,133.27 mmol) in THF (500 mL) was cooled to −78° C., n-BuLi (2.5 M, 133.27 mL) was added slowly at the same temperature. Mixture was stirred for 15 min at −78° C. under nitrogen atmosphere and trimethyl borate (199.90 mmol, 22.70 mL) was added at the same temperature. The resulting reaction mixture was stirred at −78° C. for 3 hours. After completion of the reaction, the reaction mixture was quenched with 1.5 N HCl (5 mL) solution and extracted with ethyl acetate (3×60 mL). The combined organic layer was washed with aqueous NaHCO₃ solution (100 mL), dried over Na₂SO₄ and concentrated under reduced pressure to get a crude residue. The crude residue was purified by flash silica gel (230-400 mesh) column using 0-20% ethyl acetate/petroleum ether as an eluent to afford [2,6-difluoro-3-[(2,2,2-trifluoroacetyl)amino]phenyl]boronic acid (3, 15 g, 44.62 mmol, 33% yield) as a brown solid. UPLC-MS (ES−): m/z 268.0 [M−H]⁻.

### Step 3

Into a 500 mL two-neck round bottom flask containing a well-stirred solution of [2,6-difluoro-3-[(2,2,2-trifluoroacetyl)amino]phenyl]boronic acid (3, 18 g, 66.93 mmol) in AcOH (15 mL) and THF (226 mL) was added hydrogen peroxide (1.34 mol, 137.98 mL, 30% purity) at 0° C. The reaction mixture was stirred at room temperature for 16 hours. After completion of the reaction, the reaction mixture was diluted with ethyl acetate (2×300 mL) washed with water (200 mL) and saturated NaCl (200 mL), dried over anhydrous Na₂SO₄, filtered and concentrated under reduced pressure to get a crude residue. The crude residue was purified by flash silica gel (230-400 mesh) column using 25% ethyl acetate/petroleum ether as an eluent to afford N-(2,4-difluoro-3-hydroxy-phenyl)-2,2,2-trifluoro-acetamide (4, 15 g, 57.23 mmol, 86% yield) as a brown solid. UPLC-MS (ES−): m/z 240.0 [M−H]⁻.

### Step 4

Into a 500 mL two neck round bottom flask containing a well-stirred solution of N-(2,4-difluoro-3-hydroxy-phenyl)-2,2,2-trifluoro-acetamide (4, 53.92 mmol) in N,N-dimethylformamide (130 mL), were added Cs₂CO₃ (43.92 g, 134.79 mmol) and methyl 5-fluoro-2-nitro-benzoate (5, 59.31 mmol) at room temperature under nitrogen atmosphere. The reaction mixture was stirred at 50° C. for 5 hours. After completion of the reaction, the reaction mixture was diluted with water (100 mL) and extracted with ethyl acetate (2×100 mL). The combined organic layers were washed with water (100 mL) and saturated NaCl (100 mL), dried over anhydrous Na₂SO₄, filtered and concentrated under reduced pressure to get a crude residue. The crude residue was purified by flash silica gel (230-400 mesh) column using 30% ethyl acetate/petroleum ether as an eluent to afford methyl 5-[2,6-difluoro-3-[(2,2,2-trifluoroacetyl)amino]phenoxy]-2-nitro-benzoate (6, 13 g, 28.71 mmol, 53% yield) as a brown liquid. UPLC-MS (ES−): m/z 419.0 [M−H]⁻.

### Step 5

A 500 mL single-neck round-bottom flask containing a well-stirred solution of methyl 5-[2,6-difluoro-3-[(2,2,2-trifluoroacetyl)amino]phenoxy]-2-nitro-benzoate (6, 10 g, 23.80 mmol) in ethyl acetate (100 mL) was degassed with nitrogen gas for 5 min and palladium, 10% on carbon, type 487, dry (5.00 g, 46.98 mmol) was added. Reaction mixture was stirred under hydrogen (bladder) atmosphere at room temperature for 4 hours. After completion of the reaction, the reaction mixture was filtered through celite and the filtrate was concentrated under reduced pressure to afford crude methyl 2-amino-5-[2,6-difluoro-3-[(2,2,2-trifluoroacetyl)amino]phenoxy]benzoate (7, 9 g, 16.14 mmol, 68% yield) as an off-white solid. UPLC-MS (ES+): m/z 391.0 [M+H]⁺.

### Step 6

Into a 1000 mL two-neck round-bottom flask containing a well-stirred solution of methyl 2-amino-5-[2,6-difluoro-3-[(2,2,2-trifluoroacetyl)amino]phenoxy]benzoate (7, 10 g, 25.62 mmol) and tert-butyl 3-amino-1-oxa-8-azaspiro[4.5]decane-8-carboxylate (8, 6.57 g, 25.62 mmol) in a mixture of THF (35 mL) and toluene (100 mL) was added triethyl orthoformate (51.25 mmol, 8.52 mL) at room temperature under nitrogen atmosphere. The reaction mixture was stirred at 110° C. for 24 hours. After completion of the reaction, the reaction mixture was diluted with water (40 mL) and extracted with ethyl acetate (2×50 mL). The combined organic layers were washed with NaHCO₃ solution (40 mL), saturated NaCl (30 mL), dried over anhydrous Na₂SO₄, filtered and concentrated under reduced pressure to get a crude residue. The crude residue was purified by flash silica gel (230-400 mesh) column using 20% ethyl acetate/petroleum ether as an eluent to afford tert-butyl 3-[6-[2,6-difluoro-3-[(2,2,2-trifluoroacetyl)amino]phenoxy]-4-oxo-quinazolin-3-yl]-1-oxa-8-azaspiro[4.5]decane-8-carboxylate (9, 3.8 g, 5.11 mmol, 20% yield) as a brown solid. UPLC-MS (ES+): m/z 625.2 [M+H]⁺.

### Step 7

Into a 50 mL two-neck round-bottom flask containing a well stirred solution of tert-butyl 3-[6-[2,6-difluoro-3-[(2,2,2-trifluoroacetyl)amino]phenoxy]-4-oxo-quinazolin-3-yl]-1-oxa-8-azaspiro[4.5]decane-8-carboxylate (9, 3.5 g, 5.60 mmol) in EtOH (35 mL) was added triethylamine (56.04 mmol, 7.81 mL) at room temperature under nitrogen atmosphere. Reaction mixture was stirred at 80° C. for 4 hours. After completion of the reaction, the reaction mixture was diluted with water (30 mL) and extracted with ethyl acetate (3×30 mL). The combined organic layers were washed with cold water (2×30 mL), dried over anhydrous Na₂SO₄, filtered and concentrated under reduced pressure to afford crude tert-butyl 3-[6-(3-amino-2,6-difluoro-phenoxy)-4-oxo-quinazolin-3-yl]-1-oxa-8-azaspiro[4.5]decane-8-carboxylate (10, 2.96 g, 3.08 mmol, 55% yield) as brown sticky solid. UPLC-MS (ES+): m/z 529.2 [M+H]⁺.

### Step 8

Into a 50 mL two-neck round-bottom flask containing a well-stirred solution of tert-butyl 3-[6-(3-amino-2,6-difluoro-phenoxy)-4-oxo-quinazolin-3-yl]-1-oxa-8-azaspiro[4.5]decane-8-carboxylate (10, 1.5 g, 2.58 mmol) in 1,4-dioxane (15 mL) was added pyridine (25.83 mmol, 2.08 mL) followed by the addition of N-ethyl-N-methyl-sulfamoyl chloride (11, 15.50 mmol, 1.91 mL) at room temperature under nitrogen atmosphere. The reaction mixture was stirred at 100° C. for 16 hours. After completion of the reaction, the raction mixture was diluted with ethyl acetate (50 mL), washed with water (40 mL), saturated NaCl (40 mL), dried over Na₂SO₄ and concentrated under reduced pressure to get a crude residue. The crude residue was purified by flash silica gel (230-400 mesh) column using 50-60% ethyl acetate/petroleum ether as an eluent to afford a crude residue which was purified by achiral SFC to afford tert-butyl 3-[6-[3-[[ethyl(methyl)sulfamoyl]amino]-2,6-difluoro-phenoxy]-4-oxo-quinazolin-3-yl]-1-oxa-8-azaspiro[4.5]decane-8-carboxylate (12, 1.4 g, 2.09 mmol, 81% yield) as a pale yellow solid.

### Method of Analysis for Achiral SFC Separation

Instrument: S/DC/ARD/22-020
Column: LUX-A1, 0.5% IPAm in IPA
Mobile Phase: 0.5% IPAm in IPA
Total Flow: 4 mL/min
Back pressure: 100 bar
Wavelength: 220 nm
Cycle time: 8 min
UPLC-MS (ES+): m/z 648.2 [M−H]⁻.

Step 9: Chiral SFC separation: Above isomeric mixture of tert-butyl 3-[6-[3-[[ethyl(methyl)sulfamoyl]amino]-2,6-difluoro-phenoxy]-4-oxo-quinazolin-3-yl]-1-oxa-8-azaspiro[4.5]decane-8-carboxylate (12, 1.4 g, 2.09 mmol, 81% yield) was purified by chiral SFC following the below method.

### Method of Analysis for Chiral SFC Separation

Instrument: S/DC/ARD/22-020
Column: LUX-A¹, 0.5% IPAm in IPA
Mobile Phase: 0.5% IPAm in IPA
Total Flow: 4 mL/min
Back pressure: 100 bar
Wavelength: 220 nm
Cycle time: 8 min

Fast eluting isomer (Peak-1): Fast eluting compound (Peak-1, t_{R}=3.28 min) was collected and concentrated under reduced pressure to afford tert-butyl(3R)-3-[6-[3-[[ethyl(methyl)sulfamoyl]amino]-2,6-difluoro-phenoxy]-4-oxo-quinazolin-3-yl]-1-oxa-8-azaspiro[4.5]decane-8-carboxylate (12a, 200 mg, 227.80 mol, 11% yield) as an off-white solid. Peak-1 was designated as an S-isomer based on preliminary data. UPLC-MS (ES+): m/z 594.2 [M−Bu+H]⁺.

Late eluting isomer (Peak-2): Late eluting compound (Peak-2, t_{R}=4.28 min) was collected and concentrated under reduced pressure to afford tert-butyl(3R)-3-[6-[3-[[ethyl(methyl)sulfamoyl]amino]-2,6-difluoro-phenoxy]-4-oxo-quinazolin-3-yl]-1-oxa-8-azaspiro[4.5]decane-8-carboxylate (12b, 400 mg, 615.66 mol, 29% yield) as an off-white solid. Peak-2 was designated as an R-isomer based on preliminary data. UPLC-MS (ES+): m/z 594.2 [M−^{t}Bu+H]⁺.

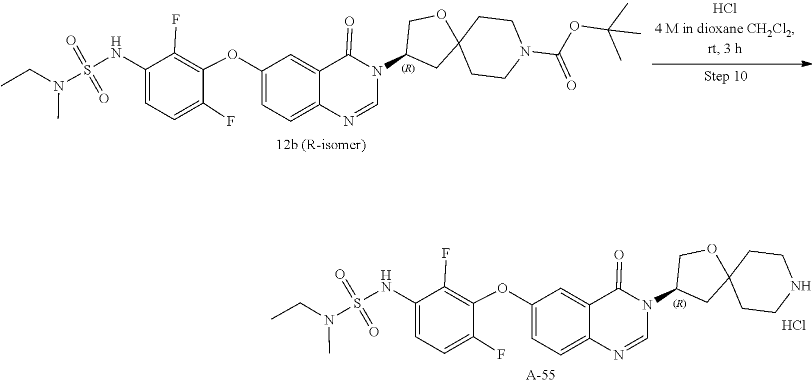

### Step 10

Into a 50 mL single-neck round-bottom flask containing a well-stirred solution of tert-butyl(3R)-3-[6-[3-[[ethyl(methyl)sulfamoyl]amino]-2,6-difluoro-phenoxy]-4-oxo-quinazolin-3-yl]-1-oxa-8-azaspiro[4.5]decane-8-carboxylate (12b, 0.2 g, 307.83 mol) in dichloromethane (3 mL) was added HCl (4M in 1,4-dioxane, 2 mL) slowly at 0° C. under nitrogen atmosphere. The resulting solution was stirred at room temperature for 3 hours. After completion of the reaction, the reaction mixture was concentrated under reduced pressure and the obtained residue was co-distilled with dichloromethane (2×5 mL) to afford 3-[6-[3-[[ethyl(methyl)sulfamoyl]amino]-2,6-difluoro-phenoxy]-4-oxo-quinazolin-3-yl]-1-oxa-8-azaspiro[4.5]decane (A-55, 150 mg, 254.90 mol, 83% yield, HCl salt) as an off-white solid. UPLC-MS (ES+): m/z 550.0 [M+H]⁺.

### Synthesis A56: Synthesis of 4-(2-azaspiro[3.3]heptan-6-yloxy)-6-[2-cyano-3-[[ethyl(methyl)sulfamoyl]amino]-6-fluoro-phenoxy]quinazoline (A-56)

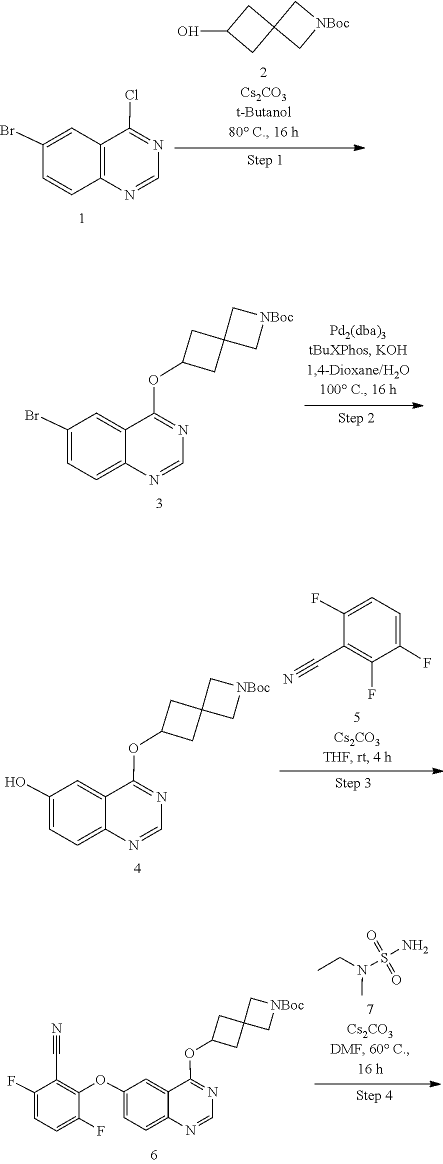

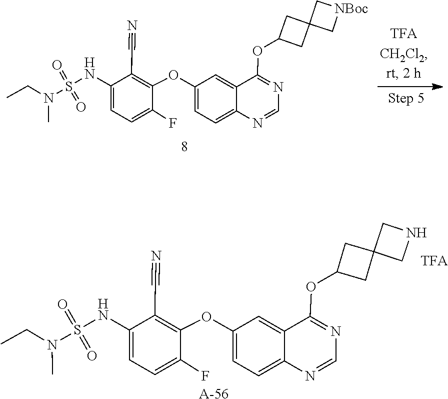

### Step 1

Into a 100 mL single neck round-bottom flask containing a well stirred solution of 6-bromo-4-chloro-quinazoline (1, 2.5 g, 10.27 mmol) in t-butanol (30 mL) were added Cs₂CO₃ (16.73 g, 51.34 mmol) and tert-butyl 6-hydroxy-2-azaspiro[3.3]heptane-2-carboxylate (2, 3.28 g, 15.40 mmol) at room temperature and resulting reaction mixture was stirred at 80° C. for 16 hours. After completion of the reaction, the reaction mixture was diluted with water (30 mL) and extracted with ethyl acetate (2×100 mL). Combined organic layer was dried over anhydrous Na₂SO₄, filtered and concentrated under reduced pressure to get crude residue which was purified by flash (230-400 silica mesh, 50 g) using 0-100% ethyl acetate/hexane as an eluent while desired product was eluting at 20-25% of mobile phase to afford tert-butyl 6-(6-bromoquinazolin-4-yl)oxy-2-azaspiro[3.3]heptane-2-carboxylate (3, 1.8 g, 4.26 mmol, 41% yield) as a light yellow solid. UPLC-MS (ES+): m/z 420.0 [M+H]⁺.

### Step 2

Into a 40 mL pressure vial containing water (4 mL), KOH (298.81 mg, 5.33 mmol, 146.19 L) was added at 0° C. and stirred for 10 min. To the reaction mixture 1,4-dioxane (12 mL), tert-butyl 6-(6-bromoquinazolin-4-yl)oxy-2-azaspiro[3.3]heptane-2-carboxylate (3, 900 mg, 2.13 mmol) followed by t-BuXPhos (90.46 mg, 213.02 mol) were added and reaction mixture was degassed with nitrogen gas for 10 min. Pd₂(dba)₃ (195.07 mg, 213.02 mol) was added and resulting reaction mixture was stirred at 100° C. for 16 hours. After completion of the reaction, the reaction mixture was diluted with ethyl acetate (100 mL) and water (30 mL). Layers were separated and organic layer was dried over anhydrous Na₂SO₄, filtered and concentrated under reduced pressure to afford crude residue which was purified by flash silica-gel (60-120 silica mesh, 25 g) using 0-100% ethyl acetate/hexane as an eluent while desired product was eluting at 35-40% of mobile phase to afford tert-butyl 6-(6-hydroxyquinazolin-4-yl)oxy-2-azaspiro[3.3]heptane-2-carboxylate (4, 600 mg, 1.54 mmol, 72% yield) as an off-white solid. UPLC-MS (ES+): m/z 358.3 [M+H]⁺.

### Step 3

Into a 100 mL single neck round-bottom flask containing a well stirred solution of tert-butyl 6-(6-hydroxyquinazolin-4-yl)oxy-2-azaspiro[3.3]heptane-2-carboxylate (4, 1 g, 2.57 mmol) in THF (20 mL) were added Cs₂CO₃ (2.52 g, 7.72 mmol) and 2,3,6-trifluorobenzonitrile (5,3.09 mmol, 356.80 μL) at 0° C. Resulting reaction mixture was stirred at room temperature for 4 hours. After completion of the reaction, the reaction mixture was diluted with water (100 mL) and extracted with ethyl acetate (2×150 mL). Combined organic layer was dried over anhydrous Na₂SO₄, filtered and filtrate was concentrated under reduced pressure to get crude residue which was purified by flash (100-200 silica mesh, 25 g) column chromatography using 0-100% ethyl acetate/hexane as an eluent while desired product was eluting at 30-40% of mobile phase to afford tert-butyl 6-[6-(2-cyano-3,6-difluoro-phenoxy)quinazolin-4-yl]oxy-2-azaspiro[3.3]heptane-2-carboxylate (6, 1.3 g, 2.56 mmol, 99% yield) as an off-white solid. UPLC-MS (ES+): m/z 495.4 [M+H]⁺.

### Step 4

Into a 40 mL glass vial with pressure released cap containing a well-stirred solution of tert-butyl 6-[6-(2-cyano-3,6-difluoro-phenoxy)quinazolin-4-yl]oxy-2-azaspiro[3.3]heptane-2-carboxylate (6, 900 mg, 1.77 mmol) in N,N-dimethylformamide (10 mL) were added Cs₂CO₃ (1.73 g, 5.32 mmol) and [methyl(sulfamoyl)amino]ethane (7, 367.46 mg, 2.66 mmol) at room temperature and resulting reaction mixture was stirred at 60° C. for 16 hours. After completion of the reaction, the reaction mixture was diluted with water (100 mL) and extracted with ethyl acetate (2×150 mL). Combined organic layer was dried over anhydrous Na₂SO₄, filtered and filtrate was concentrated under reduced pressure to get a crude residue which was purified by flash column chromatography on silica gel (100-200 mesh) using 0-100% ethyl acetate/hexane as an eluent and desired compound was eluting at 35-40% of mobile phase to afford tert-butyl 6-[6-[2-cyano-3-[[ethyl(methyl)sulfamoyl]amino]-6-fluoro-phenoxy]quinazolin-4-yl]oxy-2-azaspiro[3.3]heptane-2-carboxylate (8, 600 mg, 923.50 mol, 52% yield) as an off-white solid. UPLC-MS (ES+): m/z 613.3 [M+H]⁺.

### Step 5

Into a 50 mL single neck round-bottom flask containing a well stirred solution of tert-butyl 6-[6-[2-cyano-3-[[ethyl(methyl)sulfamoyl]amino]-6-fluoro-phenoxy]quinazolin-4-yl]oxy-2-azaspiro[3.3]heptane-2-carboxylate (8, 600 mg, 923.50 mol) in dichloromethane (4 mL) was added trifluoroacetic acid (19.60 mmol, 1.5 mL) at 0° C. Resulting reaction mixture was stirred at room temperature for 2 hours. After completion of the reaction, the reaction mixture was concentrated under reduced pressure to get crude residue which was triturated with methyl tert-butyl ether (20 mL) and dried under reduced pressure to get 4-(2-azaspiro[3.3]heptan-6-yloxy)-6-[2-cyano-3-[[ethyl(methyl)sulfamoyl]amino]-6-fluoro-phenoxy]quinazoline (A-56, 420 mg, 653.55 mol, 71% yield, TFA salt) as an off-white solid. UPLC-MS (ES+): m/z 513.3 [M+H]⁺.

### Synthesis A57: Synthesis of afford 4-(7-azaspiro[3.5]nonan-2-yloxy)-6-[2-cyano-3-[[ethyl(methyl)sulfamoyl]amino]-6-fluoro-phenoxy]quinazoline (A-57)

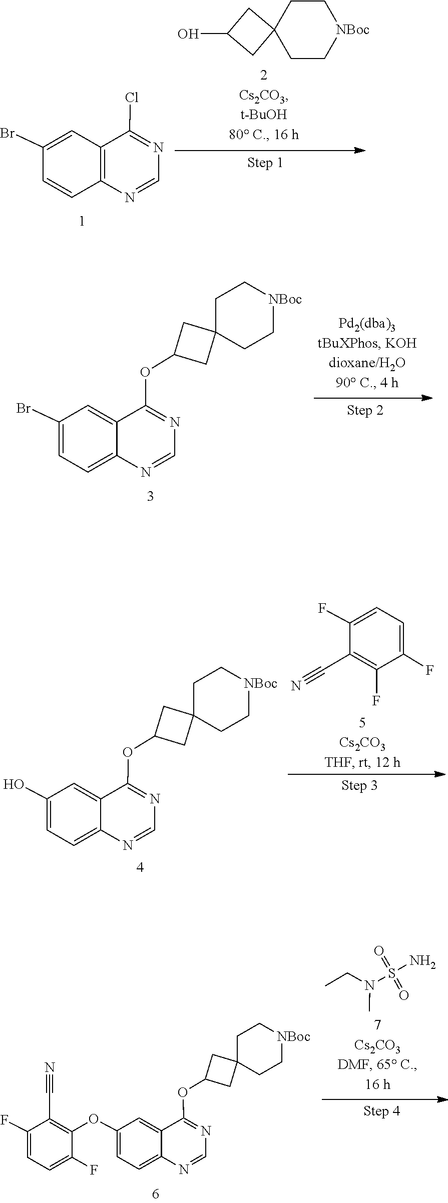

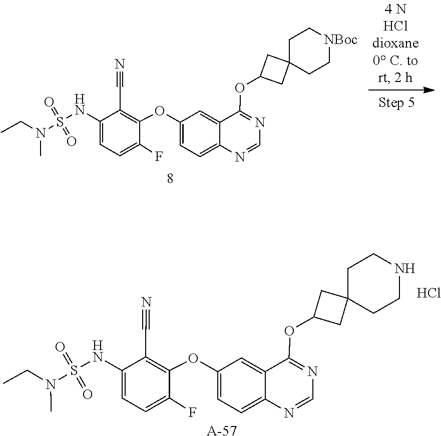

### Step 1

Into a 500 mL single neck round-bottom flask containing a well-stirred solution of 6-bromo-4-chloro-quinazoline (1, 2 g, 8.21 mmol) and tert-butyl 2-hydroxy-7-azaspiro[3.5]nonane-7-carboxylate (2, 2.18 g, 9.04 mmol) in tert-Butanol (40 mL) was added Cs₂CO₃ (16.06 g, 49.28 mmol) at room temperature and the resulting suspension was stirred at 80° C. for 16 hours. After completion of the reaction, the reaction mixture was diluted with water (150 mL) and extracted with ethyl acetate (2×90 mL). Combined organic layers were dried (anhydrous Na₂SO₄), filtered and the filtrate was concentrated under reduced pressure to get a crude residue, which was purified by flash silica-gel (230-400 mesh) column with 0-100% ethyl acetate/hexane to afford tert-butyl 2-(6-bromoquinazolin-4-yl)oxy-7-azaspiro[3.5]nonane-7-carboxylate (3, 1.6 g, 3.52 mmol, 43% yield) as a pale yellow solid. UPLC-MS (ES+): m/z 450.0 [M+2]⁺ (bromo pattern).

### Step 2

Into a 40 mL pressure vial containing a well-stirred solution of tert-butyl 2-(6-bromoquinazolin-4-yl)oxy-7-azaspiro[3.5]nonane-7-carboxylate (3, 1.6 g, 3.53 mmol) in 1,4-dioxane (25 mL) was added KOH (495.54 mg, 8.83 mmol, 242.44 L) in Water (5 mL) at ambient temperature under nitrogen atmosphere. The resulting reaction mixture was degassed by bubbling nitrogen gas into the reaction mixture for 10 min. Subsequently, ^{t}BuXPhos (150.02 mg, 353.29 mol) and Pd₂dba₃ (323.52 mg, 353.29 mol) were added, and the reaction mixture was stirred at 90° C. for 4 hours. After completion of the reaction, the reaction mixture was allowed to cool to room temperature diluted with water (100 mL) and extracted with ethyl acetate (2×90 mL). Combined organic layers were dried over anhydrous Na₂SO₄, filtered and filtrate was concentrated under reduced pressure to get crude which was purified by flash silica-gel (100-200 mesh) column with 0-100% ethyl acetate/hexane to afford tert-butyl 2-(6-hydroxyquinazolin-4-yl)oxy-7-azaspiro[3.5]nonane-7-carboxylate (4, 1 g, 2.53 mmol, 72% yield) as an off-white solid. UPLC-MS (ES+): m/z 386.3 [M+H]⁺.

### Step 3

Into a 100 mL single neck round-bottom flask containing a well-stirred solution of tert-butyl 2-(6-hydroxyquinazolin-4-yl)oxy-7-azaspiro[3.5]nonane-7-carboxylate (4, 1.2 g, 3.05 mmol) in anhydrous THF (20 mL) were added 2,3,6-trifluorobenzonitrile (5,3.66 mmol, 422.78 L) and Cs₂CO₃ (2.98 g, 9.15 mmol) at room temperature and the resulting suspension was stirred at room temperature for 12 hours. After completion of the reaction, the reaction mixture was diluted with water (40 mL) and extracted with ethyl acetate (2×80 mL). Combined organic layers were dried over anhydrous Na₂SO₄, filtered and the filtrate was concentrated under reduced pressure to get a crude residue, which was purified by flash silica-gel (230-400 mesh) column with 0-100% ethyl acetate/hexane to afford tert-butyl 2-[6-(2-cyano-3,6-difluoro-phenoxy)quinazolin-4-yl]oxy-7-azaspiro[3.5]nonane-7-carboxylate (6, 1.5 g, 2.84 mmol, 93% yield) as an off-white solid. UPLC-MS (ES+): m/z 523.4 [M+H]⁺.

### Step 4

Into a 100 mL single neck round-bottom flask containing a well-stirred solution of tert-butyl 2-[6-(2-cyano-3,6-difluoro-phenoxy)quinazolin-4-yl]oxy-7-azaspiro[3.5]nonane-7-carboxylate (6, 1.45 g, 2.72 mmol) and [methyl(sulfamoyl)amino]ethane (7, 751.58 mg, 5.44 mmol) in N,N-dimethylformamide (15 mL) was added Cs₂CO₃ (2.66 g, 8.16 mmol) at room temperature and the resulting suspension was stirred at 65° C. for 16 hours. After completion of the reaction, the reaction mixture was diluted with water (30 mL), solid was precipitated out and filtered through sintered funnel to remove fluorescent impurity. The layers were separated, and aqueous layer was extracted with ethyl acetate (2×20 mL). Combined organic layers were washed with water (20 mL) and dried over anhydrous Na₂SO₄, filtered and the filtrate was concentrated under reduced pressure to afford tert-butyl 2-[6-[2-cyano-3-[[ethyl(methyl)sulfamoyl]amino]-6-fluoro-phenoxy]quinazolin-4-yl]oxy-7-azaspiro[3.5]nonane-7-carboxylate (8, 0.650 g, 894.77 mol, 33% yield) as a brown gum. UPLC-MS (ES+): m/z 641.3 [M+H]⁺.

### Step 5

Into a 50 mL single neck round-bottom flask containing a well stirred solution of tert-butyl 2-[6-[2-cyano-3-[[ethyl(methyl)sulfamoyl]amino]-6-fluoro-phenoxy]quinazolin-4-yl]oxy-7-azaspiro[3.5]nonane-7-carboxylate (8, 350 mg, 480.71 mol) in dichloromethane (6 mL) was added 4 M HCl in 1,4-dioxane (4 M, 2 mL) slowly at 0° C. under nitrogen atmosphere. Resulting reaction mixture was stirred at room temperature for 2 hours. After completion of the reaction, the reaction mixture was concentrated under reduced pressure to get a crude residue which was washed with methyl tert-butyl ether to remove the non-polar impurity to afford 4-(7-azaspiro[3.5]nonan-2-yloxy)-6-[2-cyano-3-[[ethyl(methyl)sulfamoyl]amino]-6-fluoro-phenoxy]quinazoline (A-57, 300 mg, 405.50 mol, 84% yield, HCl salt) as a gummy liquid. UPLC-MS (ES+): m/z 541.2 [M+H]⁺.

### Synthesis A58 and A59: Synthesis of (3S)-3-[7-[2-Cyano-3-[[ethyl(methyl)sulfamoyl]amino]-6-fluoro-phenoxy]quinoxalin-2-yl]-1-oxa-8-azaspiro[4.5]decane (A-58) and (3R)-3-[7-[2-Cyano-3-[[ethyl(methyl)sulfamoyl]amino]-6-fluoro-phenoxy]quinoxalin-2-yl]-1-oxa-8-azaspiro[4.5]decane (A-59)

### Synthesis of int-4

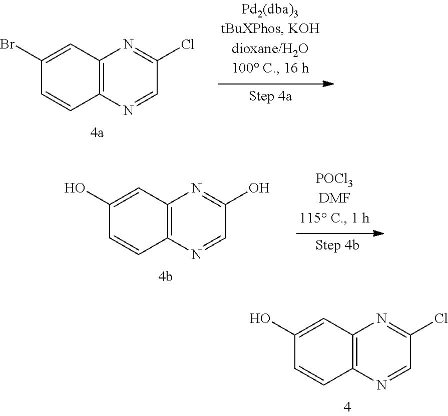 [SMILES: Clc1cnc2ccc(Br)cc2n1 ; Oc1ccc2ncc(O)nc2c1 ; Oc1ccc2ncc(Cl)nc2c1]

### Step 4a

A solution of KOH (7.20 g, 128.34 mmol, 3.52 mL) in water (25 mL) and 1,4-dioxane (100 mL) in a 250 mL sealed tube was cooled to 10° C. and 7-bromo-2-chloro-quinoxaline (4a, 12.5 g, 51.34 mmol) followed by ^{t-}BuXPhos (2.18 g, 5.13 mmol) were added. Mixture was purged with nitrogen gas for 15 min and Pd₂(dba)₃ (4.70 g, 5.13 mmol) was added at the room temperature under nitrogen atmosphere. Resulting reaction mixture was stirred at 100° C. for 16 hours. After completion of the reaction, the reaction mixture was diluted with 1M KOH solution (160 mL) and ethyl acetate (300 mL). Layers were separation and aqueous layer was acidified with aqueous 1.5N HCl solution to adjust pH 3. Precipitated solid was isolated by filtration and dried under vacuum to afford quinoxaline-2,7-diol (4b, 5.5 g, 28.83 mmol, 56% yield) as a light brown solid. UPLC-MS (ES+): m/z 163.0 [M+H]⁺.

### Step 4b

Into a 500 mL single-neck round-bottom flask containing a well-stirred solution of quinoxaline-2,7-diol (4b, 11 g, 57.66 mmol) in N,N-dimethylformamide (11 mL) was added POCl₃ (64 mmol, 53.75 mL) slowly at the room temperature and reaction mixture was stirred at 115° C. for 1 hour. After consumption of the starting material, the reaction mixture was concentrated under reduced pressure to get a crude residue. The crude material was diluted with water (200 mL) and extracted with ethyl acetate (3×200 mL). Combined organic layer was washed with saturated aqueous NaHCO₃ solution (200 mL) and saturated NaCl solution (200 mL), dried over anhydrous Na₂SO₄, filtered and concentrated under reduced pressure to afford a crude residue which was taken in methyl tert-butyl ether (200 mL) and mixture was stirred for 10 min. Undissolved solid was filtered-off through cotton pad to remove polar impurities. Filtrate was concentrated under reduced pressure to get desired product along with little non-polar impurities which was triturated with n-pentane (50 mL) and dried to afford 3-chloroquinoxalin-6-ol (4, 5.5 g, 27.58 mmol, 48% yield) as a light brown solid. UPLC-MS (ES+): m/z 181.0 [M+H]⁺.

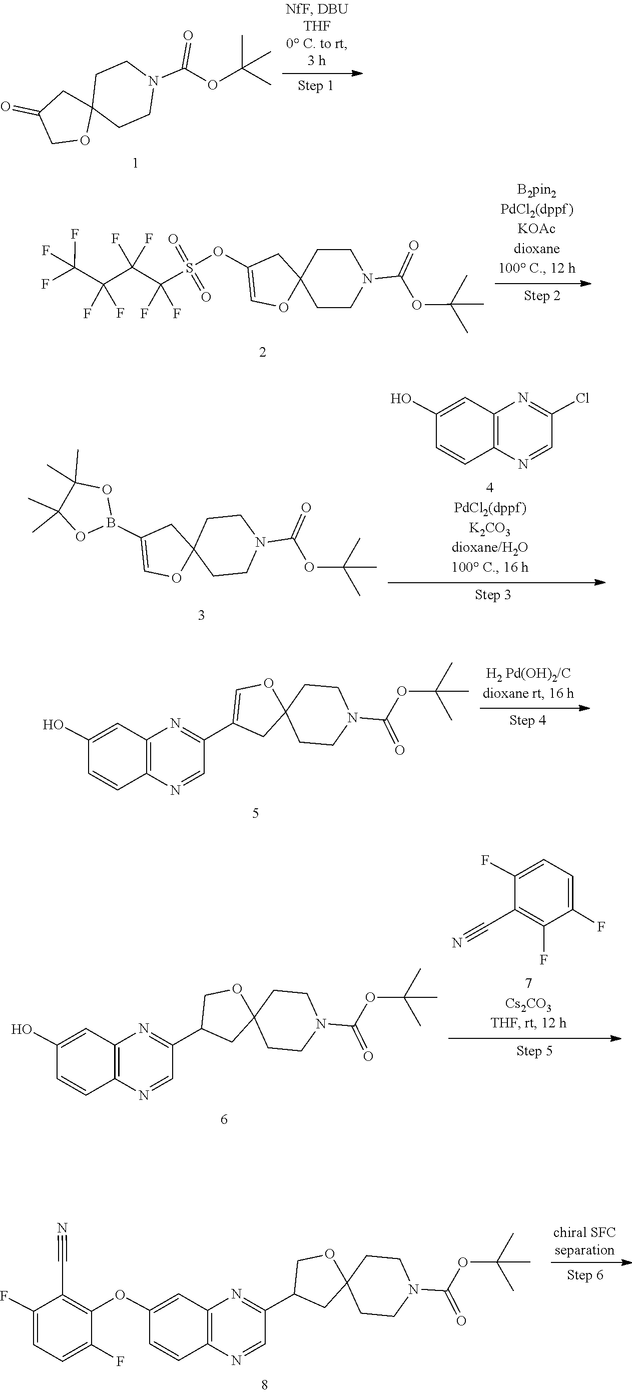

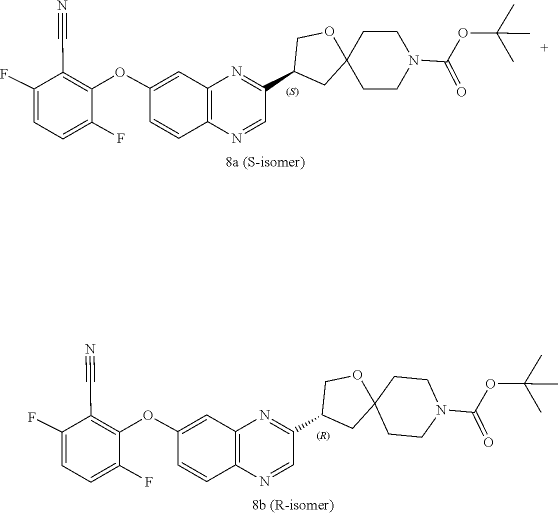

### Step 1

Into a 1000 mL single-neck round-bottom flask containing a well-stirred solution of tert-butyl 3-oxo-1-oxa-8-azaspiro[4.5]decane-8-carboxylate (1, 25 g, 97.92 mmol) in THF (300 mL) was added DBU (244.80 mmol, 36.61 mL) at 0° C. under nitrogen atmosphere and reaction mixture was stirred at 0° C. for 30 minutes and was added 1,1,2,2,3,3,4,4,4-nonafluorobutane-1-sulfonyl fluoride (146.88 mmol, 25.36 mL) at the same temperature. The resulting solution was stirred at room temperature for 3 hours. After 3 h, the reaction mixture was diluted with water (200 mL) and extracted with ethyl acetate (2×200 mL). Organic layer was separated, dried over anhydrous Na₂SO₄, filtered and concentrated under reduced pressure to get a crude residue which was purified by flash silica gel (100-200 silica-gel mesh, 320 g) column chromatography using 0-100% ethyl acetate/hexane as an eluent while compound was eluting at 10-15% of mobile phase to afford tert-butyl 3-(1,1,2,2,3,3,4,4,4-nonafluorobutylsulfonyloxy)-1-oxa-8-azaspiro[4.5]dec-2-ene-8-carboxylate (2, 21 g, 38.98 mmol, 40% yield) as a pale yellow semi-solid. UPLC-MS (ELSD) (ES+): m/z 438 [M+H−Boc]⁺.

### Step 2

Into a 500 mL single-neck round-bottom flask containing a well-stirred solution of tert-butyl 3-(1,1,2,2,3,3,4,4,4-nonafluorobutylsulfonyloxy)-1-oxa-8-azaspiro[4.5]dec-2-ene-8-carboxylate (2, 14 g, 26.05 mmol) in 1,4-dioxane (200 mL) was added KOAc (7.67 g, 78.16 mmol, 4.89 mL) followed by bis(pinacolato)diboron (7.94 g, 31.26 mmol) at room temperature. The resulting solution was purged with nitrogen gas for 5 min and [1,1′-bis(diphenylphosphino)ferrocene]dichloropalladium(II), complex with dichloromethane (2.13 g, 2.61 mmol) was added at room temperature. Resulting reaction mixture was stirred at 100° C. for 12 hours. The reaction mixture was cooled to room temperature, filtered through a celite pad and pad was washed with ethyl acetate (200 mL). Filtrate was concentrated under reduced pressure to get a crude residue which was purified by flash silica gel (100-200 mesh, 240 g) column chromatography using 0-100% ethyl acetate/hexane as an eluent to afford tert-butyl 3-(4,4,5,5-tetramethyl-1,3,2-dioxaborolan-2-yl)-1-oxa-8-azaspiro[4.5]dec-2-ene-8-carboxylate (3, 5 g, 13.28 mmol, 51% yield) as an brown thick gum. UPLC-MS (ELSD) (ES+): m/z 184.2 [M+H−Boc −2,3-dimethylbutane]⁺.

### Step 3

Into a 250 mL sealed tube containing a well-stirred solution of 3-chloroquinoxalin-6-ol (4, 5.5 g, 30.46 mmol) and tert-butyl 3-(4,4,5,5-tetramethyl-1,3,2-dioxaborolan-2-yl)-1-oxa-8-azaspiro[4.5]dec-2-ene-8-carboxylate (3, 13.35 g, 36.55 mmol) in 1,4 dioxane (80 mL) and water (20 mL) was added K2CO₃ (10.52 g, 76.14 mmol, 4.60 mL) at room temperature. The reaction solution was degassed with nitrogen for 10 min and [1,1′-Bis(diphenylphosphino)ferrocene]dichloropalladium(II), complex with dichloromethane (2.49 g, 3.05 mmol) was added at the room temperature. The resulting solution was stirred at 100° C. for 16 hours. After completion of the reaction, the resulting solution was cooled to room temperature, filtered through a celite pad and pad was washed with ethyl acetate (100 mL) and filtrate was concentrated under reduced pressure to get a crude residue. The resulting crude was purified by flash silica gel (100-200 mesh, 120 g) column chromatography using 0-100% ethyl acetate/hexane as an eluent to afford tert-butyl 3-(7-hydroxyquinoxalin-2-yl)-1-oxa-8-azaspiro[4.5]dec-2-ene-8-carboxylate (5, 6 g, 14.72 mmol, 48% yield) as a light brown solid. UPLC-MS (ES−): m/z 382.2 [M−H]⁻.

### Step 4

Into a 250 mL single-neck round-bottom flask containing a well-stirred suspension of tert-butyl 3-(7-hydroxyquinoxalin-2-yl)-1-oxa-8-azaspiro[4.5]dec-2-ene-8-carboxylate (5, 7 g, 17.17 mmol) in 1,4-dioxane (50 mL) was added Pd(OH)₂ on carbon (20 wt. %, 50% water) (3.38 g, 24.03 mmol) at room temperature for 16 h under nitrogen atmosphere. The resulting suspension was stirred at room temperature under hydrogen bladder pressure for 5 hours. After complete consumption of the starting material, the reaction mixture was filtered through a pad of Celite, and pad was washed with ethyl acetate (200 mL). The combined filtrate was concentrated under reduced pressure to get a crude residue which was purified by flash silica-gel (230-400 mesh, 240 g) column chromatography using 0-100% ethyl acetate/hexane as an eluent to afford tert-butyl 3-(7-hydroxyquinoxalin-2-yl)-1-oxa-8-azaspiro[4.5]decane-8-carboxylate (6, 4 g, 10.32 mmol, 60% yield) as an off-white solid. UPLC-MS (ES+): m/z 386.1 [M+H]⁺.

### Step 5

Into a 250 mL three-neck round-bottom flask containing a well-stirred solution of tert-butyl 3-(7-hydroxyquinoxalin-2-yl)-1-oxa-8-azaspiro[4.5]decane-8-carboxylate (6, 4 g, 10.27 mmol) in THF (50 mL) was added Cs₂CO₃ (8.37 g, 25.68 mmol) followed by 2,3,6-trifluorobenzonitrile (7, 12.33 mmol, 1.42 mL) at 0° C. The resulting reaction mixture was stirred at room temperature for 12 hours. After consumption of the starting material, the reaction mixture was diluted with water (100 mL) and extracted with ethyl acetate (2×100 mL). Combined organic layers were dried over anhydrous Na₂SO₄, filtered and concentrated under reduced pressure to get a crude residue which was purified by flash silica-gel (100-200 mesh, 120 g) column using 0-100% ethyl acetate/hexane as an eluent to afford tert-butyl 3-[7-(2-cyano-3,6-difluoro-phenoxy)quinoxalin-2-yl]-1-oxa-8-azaspiro[4.5]decane-8-carboxylate (8, 4.3 g, 8.18 mmol, 80% yield) as an off-white solid.

Step 6: Chiral SFC separation: Above isomeric mixture of tert-butyl 3-[7-(2-cyano-3,6-difluoro-phenoxy)quinoxalin-2-yl]-1-oxa-8-azaspiro[4.5]decane-8-carboxylate (8, 4.3 g, 8.15 mmol) was purified by chiral SFC following the below method.

### Method of Analysis for Chiral SFC Separation

Instrument: PIC-22-017
Column: LUXA1-(250*30) mm, 5 m
Mobile Phase: CO₂: 0.2% FA in IPA: CAN (1:1) [75:25]
Total Flow: 100 mL/min
Back pressure: 100 bar
Wavelength: 220 nm
Cycle time: 6 min

Fast eluting isomer (Peak-1): Fast eluting compound (Peak-1, t_{R}=2.079 min) was collected and concentrated under reduced pressure to afford tert-butyl (3S)-3-[7-(2-cyano-3,6-difluoro-phenoxy)quinoxalin-2-yl]-1-oxa-8-azaspiro[4.5]decane-8-carboxylate (8a, 1.8 g, 3.42 mmol, 42% yield) as an off-white solid. Peak-1 was designated as an S-isomer based on preliminary data. UPLC-MS (ES+): m/z 467.0 [M+H−t-Bu]⁺.

Late eluting isomer (Peak-2): Late eluting compound (Peak-2, t_{R}=2.97 min) was collected and concentrated under reduced pressure to afford tert-butyl(3R)-3-[7-(2-cyano-3,6-difluoro-phenoxy)quinoxalin-2-yl]-1-oxa-8-azaspiro[4.5]decane-8-carboxylate (8b, 1.6 g, 3.02 mmol, 37% yield) as an off-white solid. Peak-2 was designated as an R-isomer based on preliminary data. UPLC-MS (ES+): m/z 467.0 [M+H−t-Bu]⁺.

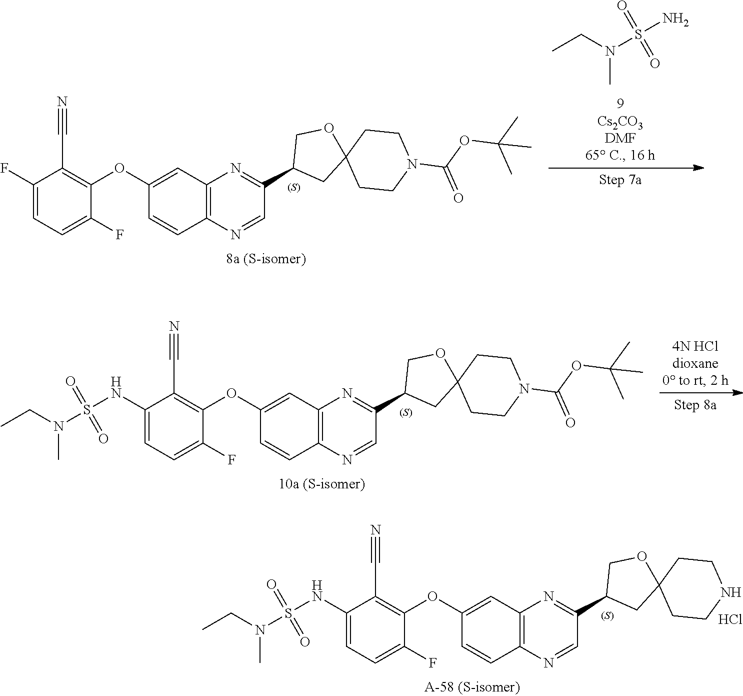

### Step 7a

Into a 20 mL glass vial with pressure released cap containing a well-stirred solution of tert-butyl (3S)-3-[7-(2-cyano-3,6-difluoro-phenoxy)quinoxalin-2-yl]-1-oxa-8-azaspiro[4.5]decane-8-carboxylate (8a, 900 mg, 1.65 mmol) in N,N-dimethylformamide (10 mL) was added [methyl(sulfamoyl)amino]ethane (9, 342.73 mg, 2.48 mmol) followed by Cs₂CO₃ (1.35 g, 4.13 mmol) at room temperature. Resulting reaction mixture was stirred at 65° C. for 16 hours. After 16 h, the reaction mixture was diluted with water (100 mL) and extracted by ethyl acetate (2×100 mL). Combined organic layers were washed with saturated NaCl solution (100 mL), dried over anhydrous Na₂SO₄, filtered and concentrated under reduced pressure to get a crude residue which was purified by flash silica gel (100-200 mesh, 120 g) column chromatography using 0-100% ethyl acetate/hexane as an eluent and compound was eluting at 40-50% of the mobile phase to afford tert-butyl (3S)-3-[7-[2-cyano-3-[[ethyl(methyl)sulfamoyl]amino]-6-fluoro-phenoxy]quinoxalin-2-yl]-1-oxa-8-azaspiro[4.5]decane-8-carboxylate (10a, 700 mg, 1.05 mmol, 64% yield) as a light brown thick gum. UPLC-MS (ES−): m/z 639.2 [M−H]⁻.

### Step 8a

Into a 100 mL single-neck round-bottom flask containing a well-stirred solution of tert-butyl (3S)-3-[7-[2-cyano-3-[[ethyl(methyl)sulfamoyl]amino]-6-fluoro-phenoxy]quinoxalin-2-yl]-1-oxa-8-azaspiro[4.5]decane-8-carboxylate (10a, 1.4 g, 2.10 mmol) in dichloromethane (15 mL) was added HCl (4.0 M in 1,4-dioxane, 5.24 mL) at 0° C. Reaction mixture was stirred at room temperature for 2 hours. After consumption of the starting material, the reaction mixture was concentrated under reduced pressure to afford (3S)-3-[7-[2-cyano-3-[[ethyl(methyl)sulfamoyl]amino]-6-fluoro-phenoxy]quinoxalin-2-yl]-1-oxa-8-azaspiro[4.5]decane (A-58, 1.1 g, 1.77 mmol, 84% yield, HCl salt) as an off-white solid. UPLC-MS (ES+): m/z 541.4 [M+H]⁺.

### Step 7b

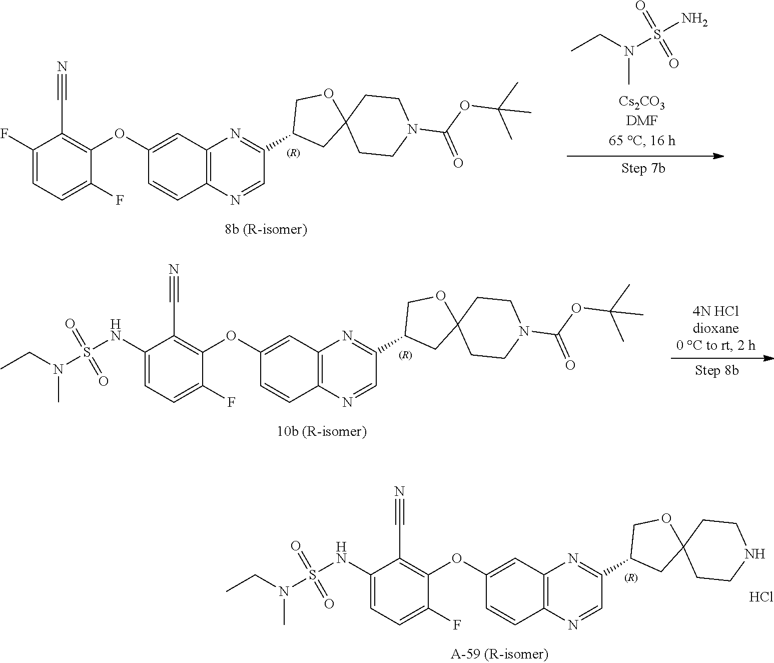

Into a 20 mL glass vial containing a well-stirred solution of tert-butyl(3R)-3-[7-(2-cyano-3,6-difluoro-phenoxy)quinoxalin-2-yl]-1-oxa-8-azaspiro[4.5]decane-8-carboxylate (8b, 850 mg, 1.53 mmol) and [methyl(sulfamoyl)amino]ethane (8b, 316.95 mg, 2.29 mmol) in N,N-dimethylformamide (10 mL) was added Cs₂CO₃ (1.25 g, 3.82 mmol) at room temperature and the resulting mixture was stirred for 16 h at 65° C. After 16 h, the reaction mixture was diluted with water (100 mL) and extracted with ethyl acetate (5×50 mL). Combined organic layers were washed with water (50 mL), dried over anhydrous Na₂SO₄, filtered and concentrated under reduced pressure to get a crude residue which was purified by flash silica-gel (230-400 mesh, 120 g) column with 0-100% ethyl acetate/hexane as an eluent while desired product was eluting at 40-60% of mobile phase to afford tert-butyl(3R)-3-[7-[2-cyano-3-[[ethyl(methyl)sulfamoyl]amino]-6-fluoro-phenoxy]quinoxalin-2-yl]-1-oxa-8-azaspiro[4.5]decane-8-carboxylate (10b, 400 mg, 589.96 mol, 39% yield) as an off-white solid. UPLC-MS (ES−): m/z 639.2 [M−H]⁻.

### Step 8b

Into a 100 mL single-neck round-bottom flask containing a well-stirred solution of tert-butyl(3R)-3-[7-[2-cyano-3-[[ethyl(methyl)sulfamoyl]amino]-6-fluoro-phenoxy]quinoxalin-2-yl]-1-oxa-8-azaspiro[4.5]decane-8-carboxylate (10b, 850 mg, 1.33 mmol) in dichloromethane (10 mL) was added HCl (4 M in 1,4-dioxane, 3.32 mL) at 0° C. under nitrogen atmosphere and the resulting mixture was stirred for 2 h at room temperature. After consumption of the starting material, the reaction mixture was concentrated directly under reduced pressure to afford (3R)-3-[7-[2-cyano-3-[[ethyl(methyl)sulfamoyl]amino]-6-fluoro-phenoxy]quinoxalin-2-yl]-1-oxa-8-azaspiro[4.5]decane (A-59, 650 mg, 1.06 mmol, 80% yield, HCl salt) as an off-white solid. UPLC-MS (ES+): m/z 541.2 [M+H]⁺.

### Synthesis A60: Synthesis of (3R)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-60)

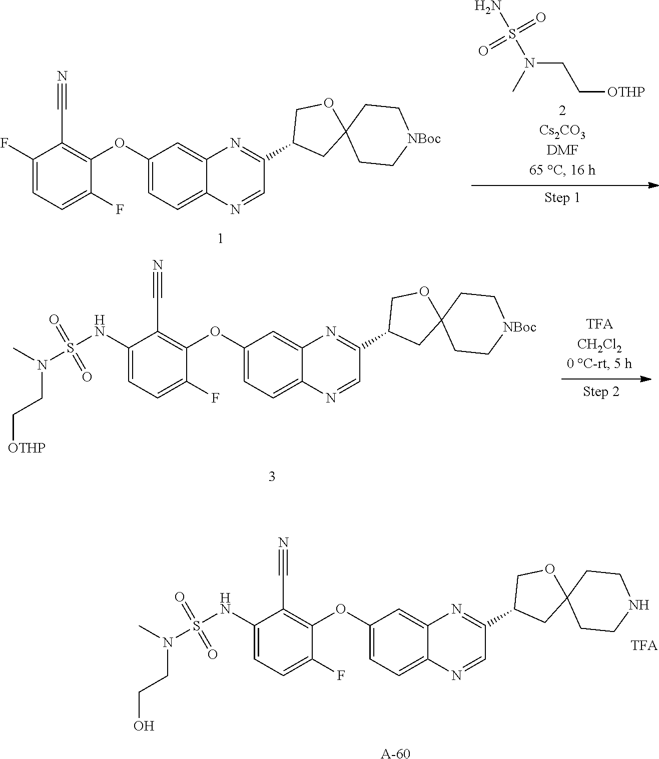

### Step 1

Into a 50 mL two-neck round-bottom flask containing a well stirred solution of tert-butyl(3R)-3-[7-(2-cyano-3,6-difluoro-phenoxy)quinoxalin-2-yl]-1-oxa-8-azaspiro[4.5]decane-8-carboxylate (1, 1.0 g, 1.89 mmol) and 2-[2-[methyl(sulfamoyl)amino]ethoxy]tetrahydropyran (2, 902.97 mg, 3.79 mmol) in N,N-dimethylformamide (15 mL) was added Cs₂CO₃ (1.54 g, 4.74 mmol) at room temperature under nitrogen atmosphere. Reaction mixture was stirred at 65° C. for 16 hours. After completion of the reaction, the reaction mixture was diluted with water (20 mL) and extracted with ethyl acetate (3×50 mL). Combined organic layer was dried over anhydrous Na₂SO₄, filtered and the filtrate was concentrated under reduced pressure to get a crude residue which was purified by flash (silica-gel 230-400 mesh, 25 g) column chromatography using 0-100% ethyl acetate/hexane as an eluent while desired product was eluted at 50-70% of the mobile phase to afford tert-butyl(3R)-3-[7-[2-cyano-6-fluoro-3-[[methyl(2-tetrahydropyran-2-yloxyethyl)sulfamoyl]amino]phenoxy]quinoxalin-2-yl]-1-oxa-8-azaspiro[4.5]decane-8-carboxylate (3, 0.700 g, 861.44 mol, 45% yield) as an off-white solid. UPLC-MS (ES−): m/z 739.2 [M−H]⁻.

### Step 2

Into a 50 mL two-neck round-bottom flask containing a well stirred solution of tert-butyl(3R)-3-[7-[2-cyano-6-fluoro-3-[[methyl(2-tetrahydropyran-2-yloxyethyl)sulfamoyl]amino]phenoxy]quinoxalin-2-yl]-1-oxa-8-azaspiro[4.5]decane-8-carboxylate (3, 1.4 g, 1.89 mmol) in dichloromethane (20 mL) was added trifluoroacetic acid (1.89 mmol, 144.61 μL) at 0° C. under nitrogen atmosphere and resulting reaction mixture was stirred for 5 h at room temperature. After completion of the reaction, the reaction mixture was concentrated under reduced pressure to afford a crude (3R)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-60, 0.800 g, 795.67 μmol, 42% yield, TFA salt) as a brown solid. UPLC-MS (ES+): m/z 557.1 [M+H]⁺.

### Synthesis A61: Synthesis of (3S)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-61)

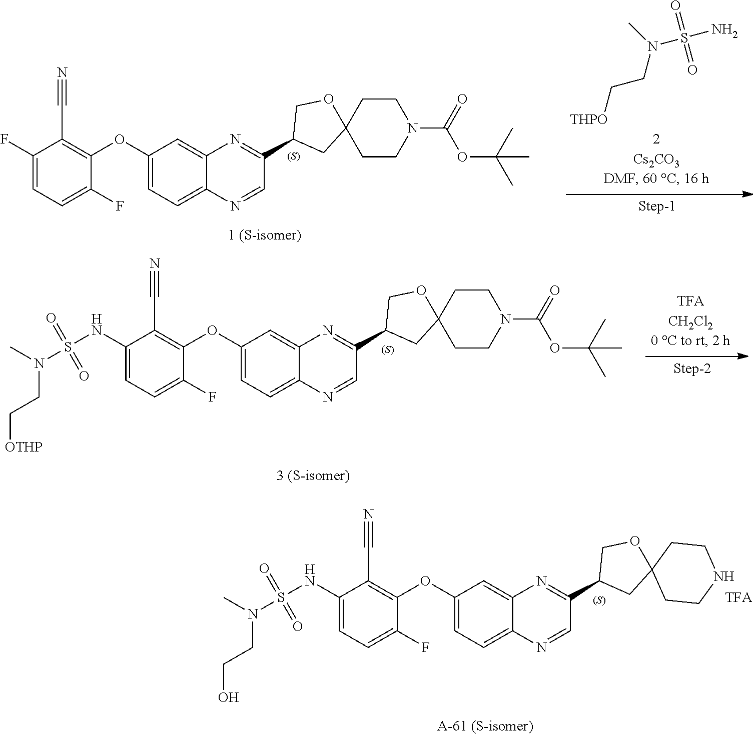

### Step 1

Into a 20 mL glass vial with pressure released cap containing a well-stirred solution of tert-butyl (3S)-3-[7-(2-cyano-3,6-difluoro-phenoxy)quinoxalin-2-yl]-1-oxa-8-azaspiro [4.5]decane-8-carboxylate (1, lg, 1.86 mmol) in N,N-dimethylformamide (10 mL) was added 2-[2-[methyl (sulfamoyl)amino]ethoxy]tetrahydropyran (2, 663.55 mg, 2.78 mmol) followed by Cs₂CO₃ (1.51 g, 4.64 mmol) at room temperature. Resulting reaction mixture stirred at 60° C. for 16 hours. After 16 h, the reaction mixture was diluted with water (100 mL) and extracted by ethyl acetate (2×100 mL). Combined organic layer was washed with saturated NaCl solution (50 mL), dried over anhydrous Na₂SO₄, filtered and concentrated under reduced pressure to get a crude residue which was purified by flash silica gel (100-200 mesh, 120 g) column chromatography using 0-100% ethyl acetate/hexane as an eluent while desired compound was eluting at 40-50% of mobile phase to afford tert-butyl (3S)-3-[7-[2-cyano-6-fluoro-3-[[methyl(2-tetrahydropyran-2-yloxyethyl)sulfamoyl]amino]phenoxy]quinoxalin-2-yl]-1-oxa-8-azaspiro[4.5]decane-8-carboxylate (3, 700 mg, 804.94 mol, 43% yield) as a light brown thick gum. UPLC-MS (ES−): m/z 739.2 [M−H]⁻.

### Step 2

Into a 100 mL single-neck round-bottom flask containing a well-stirred solution of tert-butyl (3S)-3-[7-[2-cyano-6-fluoro-3-[[methyl(2-tetrahydropyran-2-yloxyethyl)sulfamoyl]amino]phenoxy]quinoxalin-2-yl]-1-oxa-8-azaspiro[4.5]decane-8-carboxylate (3, 1.4 g, 1.89 mmol) in dichloromethane (15 mL) was added trifluoroacetic acid (28.35 mmol, 2.17 mL) at 0° C. under nitrogen atmosphere and the resulting reaction mixture was stirred at room temperature for 2 hours. After consumption of the starting material, the reaction mixture was concentrated directly under reduced pressure to get a crude residue which was triturated with methyl tert-butyl ether (2×50 mL) and dried to afford (3S)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-61, 1.1 g, 1.03 mmol, 54% yield, TFA salt) as an off-white solid. UPLC-MS (ES+): m/z 557.0 [M+H]⁺.

### Synthesis A62: Synthesis of 9-[6-[2-cyano-3-[[ethyl(methyl)sulfamoyl]amino]-6-fluoro-phenoxy]-4-oxo-quinazolin-3-yl]-3-azaspiro[5.5]undecane (A-62)

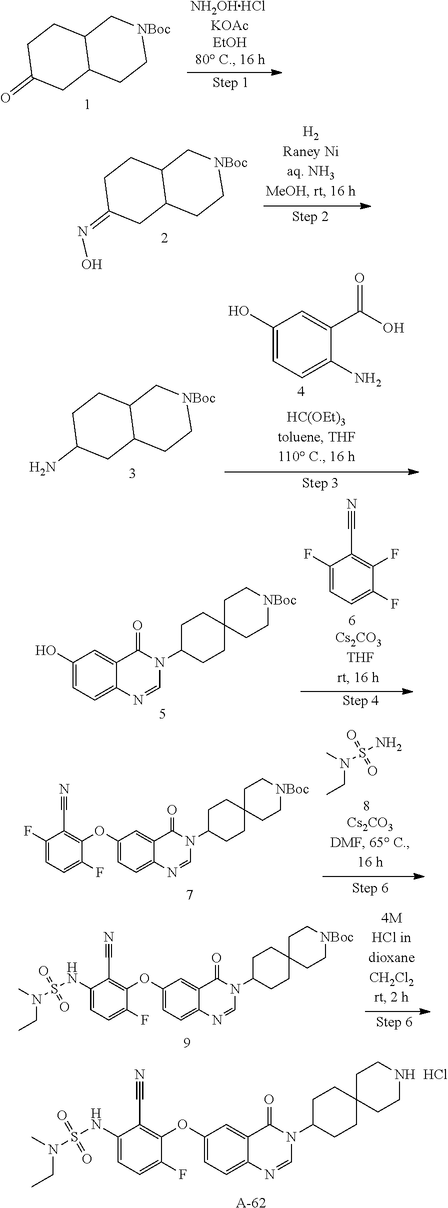

### Step 1

Into a 250 mL single-neck round-bottom flask containing a well-stirred solution of tert-butyl 9-oxo-3-azaspiro[5.5]undecane-3-carboxylate (1, 4.5 g, 16.83 mmol) in ethanol (70 mL) was added hydroxylamine hydrochloride (25.25 mmol, 1.05 mL) and potassium acetate (2.48 g, 25.25 mmol, 1.58 mL) at 0° C. Resulting reaction mixture was stirred at 80° C. for 16 hours. The reaction mixture was filtered through a celite bed and the celite bed was washed with ethyl acetate (200 mL). Filtrate was concentrated under reduced pressure to afford a crude residue which was purified by flash (silica-gel 230-400 mesh, 50 g) column chromatography using 0-100% ethyl acetate/hexane as an eluent while desired product was eluted at 20-30% of the mobile phase to afford tert-butyl 9-hydroxyimino-3-azaspiro[5.5]undecane-3-carboxylate (2, 3.8 g, 13.39 mmol, 80% yield) as an off white solid. ¹H NMR (400 MHz, DMSO-d₆): δ 6.71 (s, 1H), 3.44-3.41 (m, 4H), 2.56-2.53 (m, 2H), 2.28-2.25 (m, 2H), 1.49-1.48 (m, 13H).

### Step 2

Into a 250 mL single-neck round-bottom flask containing a well-stirred solution of tert-butyl 9-hydroxyimino-3-azaspiro[5.5]undecane-3-carboxylate (2, 3.8 g, 13.39 mmol) in methanol (80 mL) was degassed with nitrogen gas for 10 min. To the above solution, aqueous ammonia (13.39 mmol, 7 mL) and Raney Nickel® 2800 (slurry in water, active catalyst) (2.45 g, 28.55 mmol) were added at room temperature and resulted reaction mixture was stirred at the same temperature for 16 hours. The reaction mixture was filtered through Celite bed and Celite bed washed with methanol (500 mL). Filtrate was concentrated under reduced pressure and co-distilled with toluene (2×50 mL) to afford tert-butyl 9-amino-3-azaspiro[5.5]undecane-3-carboxylate (3, 3.5 g, 12.76 mmol, 95% yield) as a pale-yellow gum. UPLC-MS (ES+): m/z 269.1 [M+H]⁺.

### Step 3

Into 20 mL glass vial with pressure released cap containing a well-stirred solution of 2-amino-5-hydroxy-benzoic acid (4, 1 g, 6.53 mmol) in toluene (15 mL) and THF (5 mL) was added tert-butyl 9-amino-3-azaspiro[5.5]undecane-3-carboxylate (3, 1.75 g, 6.53 mmol) and triethyl orthoformate (6.53 mmol, 1.09 mL) at room temperature. Resulting reaction mixture was stirred at 110° C. for 16 hours. After completion of reaction, the reaction mixture was quenched with water (20 mL) and extracted with ethyl acetate (3×50 mL). The combined organic layer was washed with saturated sodium bicarbonate solution (20 mL) followed by saturated NaCl solution (20 mL), dried over anhydrous Na₂SO₄, filtered and concentrated under reduced pressure to afford a crude residue which was purified by flash (silica-gel 230-400 mesh, 50 g) column chromatography using 0-100% ethyl acetate/hexane as an eluent to afford tert-butyl 9-(6-hydroxy-4-oxo-quinazolin-3-yl)-3-azaspiro[5.5]undecane-3-carboxylate (5, 1.2 g, 2.41 mmol, 37% yield) as a brown colored solid. UPLC-MS (ES+): m/z 414.2 [M+H]⁺.

### Step 4

Into a 100 mL two-neck round-bottom flask containing a well-stirred solution of tert-butyl 9-(6-hydroxy-4-oxo-quinazolin-3-yl)-3-azaspiro[5.5]undecane-3-carboxylate (5, 1.6 g, 3.67 mmol) in THF (30 mL) was added 2,3,6-trifluorobenzonitrile (6, 4.40 mmol, 508.50 L) and cesium carbonate (3.59 g, 11.01 mmol) at room temperature and resulting reaction mixture was stirred at the same temperature for 16 hours. After completion of reaction, the reaction mixture was quenched with water (20 mL) and extracted with ethyl acetate (3×60 mL). The combined organic layer was washed with saturated NaCl solution (20 mL), dried over anhydrous Na₂SO₄, filtered and concentrated under reduced pressure to afford a crude which was purified by flash (silica-gel 230-400 mesh, 25 g) column chromatography using 0-100% ethyl acetate/hexane as an eluent while desired product was eluted at 25-100% of the mobile phase to afford tert-butyl 9-[6-(2-cyano-3,6-difluoro-phenoxy)-4-oxo-quinazolin-3-yl]-3-azaspiro[5.5]undecane-3-carboxylate (7, 1.5 g, 2.68 mmol, 73% yield) as an off white solid. UPLC-MS (ES+): m/z 551.2 [M+H]⁺.

### Step 5

Into a 100 mL two-neck round-bottom flask containing a well-stirred solution of tert-butyl 9-[6-(2-cyano-3,6-difluoro-phenoxy)-4-oxo-quinazolin-3-yl]-3-azaspiro[5.5]undecane-3-carboxylate (7, 1.5 g, 2.68 mmol) in N,N-dimethylformamide (30 mL) were added cesium carbonate (2.62 g, 8.05 mmol) and [methyl(sulfamoyl)amino]ethane (8, 741.87 mg, 5.37 mmol) at room temperature. Resulting reaction mixture was stirred at 65° C. for 16 hours. After completion of the reaction, the reaction mixture was quenched with water (50 mL), solid precipitated was filtered through Celite pad and Celite pad was washed with water (30 mL). The filtrate was extracted with ethyl acetate (3×100 mL) and the combined organic layer was washed with water (50 mL), saturated NaCl solution (50 mL), dried over anhydrous sodium sulfate, filtered and concentrated under reduced pressure to afford a crude tert-butyl 9-[6-[2-cyano-3-[[ethyl(methyl)sulfamoyl]amino]-6-fluoro-phenoxy]-4-oxo-quinazolin-3-yl]-3-azaspiro[5.5]undecane-3-carboxylate (9, 900 mg, 1.30 mmol, 49% yield) as a pale brown colored gum. UPLC-MS (ES+): m/z 669.2 [M+H]⁺.

### Step 6

Into a 100 mL single-neck round-bottom flask containing a well-stirred solution of tert-butyl 9-[6-[2-cyano-3-[[ethyl(methyl)sulfamoyl]amino]-6-fluoro-phenoxy]-4-oxo-quinazolin-3-yl]-3-azaspiro[5.5]undecane-3-carboxylate (9, 900 mg, 1.30 mmol) in dichloromethane (20 mL) was added hydrogen chloride solution 4 M in 1,4-dioxane (131.65 mmol, 6 mL) at 0° C. Resulting reaction mixture was stirred at room temperature for 2 hours. After completion of the reaction, the reaction mixture was concentrated under reduced pressure to afford a crude 9-[6-[2-cyano-3-[[ethyl(methyl)sulfamoyl]amino]-6-fluoro-phenoxy]-4-oxo-quinazolin-3-yl]-3-azaspiro[5.5]undecane (A-62, 750 mg, 1.07 mmol, 82% yield, HCl salt) as an off-white solid. UPLC-MS (ES+): m/z 569.3 [M+H]⁺.

### Synthesis A63: Synthesis of 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-63)

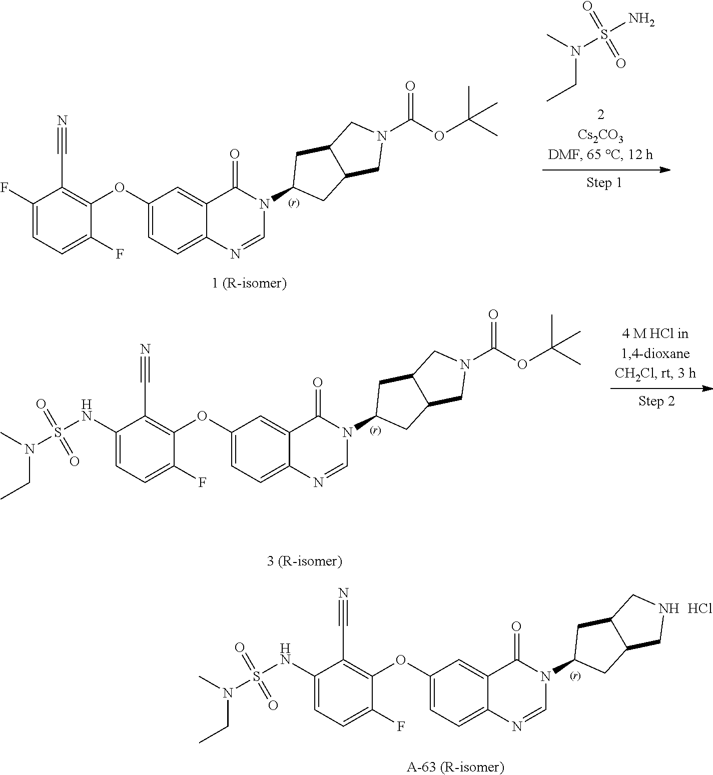

### Step 1

Into a 250 mL double-neck round-bottom flask containing a well-stirred solution of tert-butyl 5-[6-(2-cyano-3,6-difluoro-phenoxy)-4-oxo-quinazolin-3-yl]-3,3a,4,5,6,6a-hexahydro-1H-cyclopenta[c]pyrrole-2-carboxylate (1, 3.5 g, 6.88 mmol; see the synthesis of A64, intermediate 5b) in N,N-dimethylformamide (50 mL) was added cesium carbonate (6.73 g, 20.65 mmol) followed by [methyl(sulfamoyl)amino]ethane (2, 1.90 g, 13.77 mmol) at room temperature. Resulting reaction mixture was stirred at 65° C. for 12 hours. After completion of the reaction, the reaction mixture was quenched with water (150 mL), solid precipitated was filtered through celite pad and celite pad was washed with water (50 mL). The filtrate was extracted with ethyl acetate (3×100 mL) and the combined organic layer was washed with water (50 mL), saturated NaCl solution (50 mL), dried over anhydrous sodium sulfate, filtered and concentrated under reduced pressure to afford tert-butyl 5-[6-[2-cyano-3-[[ethyl(methyl)sulfamoyl]amino]-6-fluoro-phenoxy]-4-oxo-quinazolin-3-yl]-3,3a,4,5,6,6a-hexahydro-1H-cyclopenta[c]pyrrole-2-carboxylate (3, 3 g, 4.42 mmol, 64% yield) as a brown gum. UPLC-MS (ES+): m/z 627.2 [M+H]⁺.

### Step 2

Into a 100 mL single neck round bottom flask containing a well stirred solution of tert-butyl 5-[6-[2-cyano-3-[[ethyl(methyl)sulfamoyl]amino]-6-fluoro-phenoxy]-4-oxo-quinazolin-3-yl]-3,3a,4,5,6,6a-hexahydro-1H-cyclopenta[c]pyrrole-2-carboxylate (3, 3 g, 4.79 mmol) in dichloromethane (40 mL) was added hydrogen chloride solution 4 M in dioxane (11.97 mL) at 0° C. Resulting reaction mixture was stirred at room temperature for 3 hours. After completion of the reaction, the reaction mixture was concentrated under reduced pressure to afford a crude residue which was triturated with n-pentane (2×100 mL) and dried under reduced pressure to afford 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-63, 2.5 g, 3.55 mmol, 74% yield, HCl salt) as an off-white solid.

UPLC-MS(ES+): m/z 527.2 [M+H]⁺.

### Synthesis A64: Synthesis of 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-64)

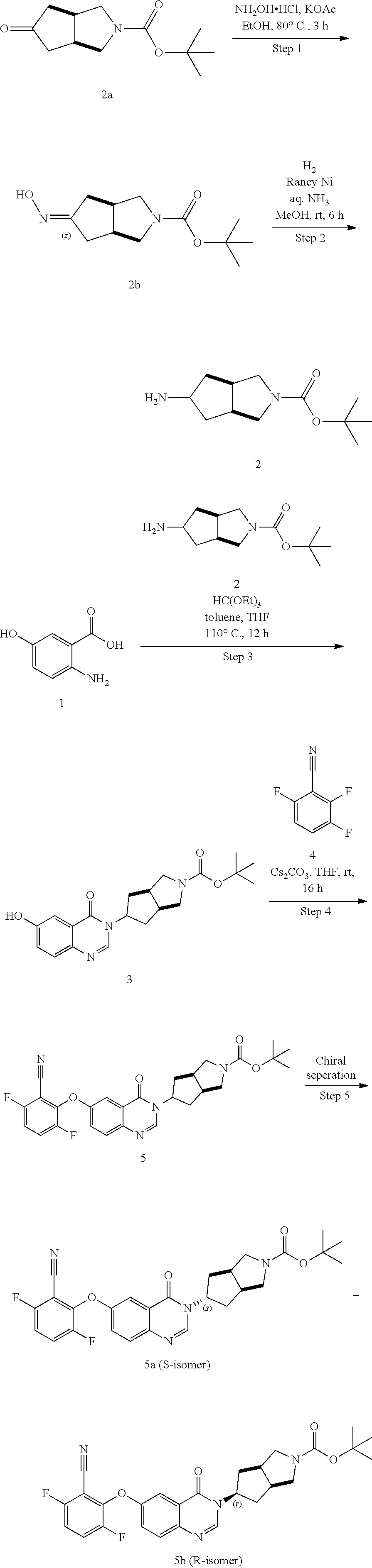

### Step 1

Into a 500 mL double-neck round-bottom flask containing a well-stirred solution of tert-butyl 5-oxo-1,3,3a,4,6,6a-hexahydrocyclopenta[c]pyrrole-2-carboxylate (2a, 10 g, 44.39 mmol) in ethanol (150 mL) was added hydroxylamine hydrochloride (4.63 g, 66.58 mmol, 2.77 mL) followed by KOAc (6.53 g, 66.58 mmol, 4.16 mL) at room temperature. Resulting reaction mixture was stirred at 80° C. for 3 hours. After completion of the reaction, the reaction mixture was filtered through a celite pad and celite pad was washed with ethyl acetate (200 mL). The filtrate was concentrated under reduced pressure to get a crude residue which was diluted with dichloromethane (200 mL) and again filtered through a celite pad and celite pad was washed with dichloromethane (200 mL). Filtrate was concentrated under reduced pressure to afford tert-butyl 5-hydroxyimino-1,3,3a,4,6,6a-hexahydrocyclopenta[c]pyrrole-2-carboxylate (2b, 10.5 g, 43.46 mmol, 98% yield) as a colorless liquid. UPLC-MS (ES+): m/z 141.1 [M−Boc]⁺.

### Step 2

Into a 1000 mL auto-clave containing a well-stirred solution tert-butyl 5-hydroxyimino-1,3,3a,4,6,6a-hexahydrocyclopenta[c]pyrrole-2-carboxylate (2b, 10.5 g, 43.70 mmol) in methanol (150 mL) was added Raney®—Nickel slurry, in H₂O, active catalyst (7.97 g, 93.07 mmol) followed by 25% aqueous ammonia (43.70 mmol, 22 mL) at room temperature. The resulting mixture was stirred at room temperature under hydrogen atmosphere (5 kg pressure), the reaction mixture was filtered through a celite pad, and celite pad was washed with methanol (200 mL). The filtrate was concentrated under reduced pressure to afford tert-butyl 5-amino-3,3a,4,5,6,6a-hexahydro-1H-cyclopenta[c]pyrrole-2-carboxylate (2, 9.75 g, 42.96 mmol, 98% yield) as a light green semi-solid. UPLC-MS (ES+): m/z 227.3 [M+H]⁺.

### Step 3

Into a 500 mL double-neck round-bottom flask containing a well-stirred solution of 2-amino-5-hydroxy-benzoic acid (1, 5.5 g, 35.92 mmol) and tert-butyl 5-amino-3,3a,4,5,6,6a-hexahydro-1H-cyclopenta[c]pyrrole-2-carboxylate (2, 9.75 g, 43.10 mmol) in toluene (100 mL) and THF (30 mL) was added triethyl orthoformate (53.87 mmol, 8.96 mL) at room temperature. Resulting reaction mixture was stirred at 110° C. for 12 hours. After completion of the reaction, the reaction mixture was quenched with water (200 mL) and extracted with ethyl acetate (2×200 mL). The combined organic layer was washed with saturated NaHCO₃ solution (200 mL), dried over anhydrous Na₂SO₄, filtered, and concentrated under reduced pressure to afford tert-butyl 5-(6-hydroxy-4-oxo-quinazolin-3-yl)-3,3a,4,5,6,6a-hexahydro-1H-cyclopenta[c]pyrrole-2-carboxylate (3, 11 g, 25.31 mmol, 70% yield) as an off-white solid. UPLC-MS (ES+): m/z 372.2 [M+H]⁺.

### Step 4

Into a 500 mL double-neck round-bottom flask containing a well-stirred solution of tert-butyl 5-(6-hydroxy-4-oxo-quinazolin-3-yl)-3,3a,4,5,6,6a-hexahydro-1H-cyclopenta[c]pyrrole-2-carboxylate (3, 11.00 g, 29.62 mmol) in THF (200 mL) was added Cs₂CO₃ (28.95 g, 88.85 mmol) followed by 2,3,6-trifluorobenzonitrile (4, 35.54 mmol, 4.11 mL) at room temperature and resulting reaction mixture was stirred at the same temperature for 16 hours. After completion of the reaction, the reaction mixture was diluted with water (200 mL) and extracted with ethyl acetate (2×200 mL). The combined organic layer was washed with saturated NaCl solution (200 mL), dried over anhydrous Na₂SO₄ and filtered, concentrated under reduced pressure to get a crude residue which was purified by flash column chromatography (230-400 mesh, 220 g) used 0-10% methanol/dichloromethane as an eluent while desired product was eluting at 5-7% of mobile phase to afford tert-butyl 5-[6-(2-cyano-3,6-difluoro-phenoxy)-4-oxo-quinazolin-3-yl]-3,3a,4,5,6,6a-hexahydro-1H-cyclopenta[c]pyrrole-2-carboxylate (5, 10 g, 18.76 mmol, 63% yield) as an off-white solid. UPLC-MS (ES+): m/z 453.0 [M-t-Bu]⁺.

Step 5: Chiral SFC separation: Above isomeric mixture of tert-butyl 5-[6-(2-cyano-3,6-difluoro-phenoxy)-4-oxo-quinazolin-3-yl]-3,3a,4,5,6,6a-hexahydro-1H-cyclopenta[c]pyrrole-2-carboxylate (5, 10 g, 18.76 mmol) was purified by chiral SFC following the below method.

### Method of Analysis for Chiral SFC Separation

Instrument: PIC 400
Column: I Cellulose B-(250*30)mm, 5 μm
Mobile Phase: CO₂: 0.5% IPAm in IPA[70:30]
Total Flow: 120 mL/min
Back pressure: 100 bar
Wavelength: 220 nm
Cycle time: 4 min

Fast eluting isomer (Peak-1): Fast eluting compound (Peak-1, t_{R}=2.48 min) was collected and concentrated under reduced pressure to afford tert-butyl (3aR,5s,6aS)-5-(6-(2-cyano-3,6-difluorophenoxy)-4-oxoquinazolin-3(4H)-yl)hexahydrocyclopenta[c]pyrrole-2(1H)-carboxylate (5a, 5 g, 9.42 mmol, 48% yield) as an off-white solid. Peak-1 was designated as an S-isomer based on preliminary data.

UPLC-MS (ES+): m/z 467.0 [M−t-Bu]⁺.

Late eluting isomer (Peak-2): Late eluting compound (Peak-2, t_{R}=2.8 min) was collected and concentrated under reduced pressure to afford tert-butyl (3aR,5r,6aS)-5-(6-(2-cyano-3,6-difluorophenoxy)-4-oxoquinazolin-3(4H)-yl)hexahydrocyclopenta[c]pyrrole-2(1H)-carboxylate (5b, 3.5 g, 6.74 mmol, 34% yield) as an off-white solid. Peak-2 was designated as an R-isomer based on preliminary data. UPLC-MS (ES+): m/z 509.1 [M+H]⁺.

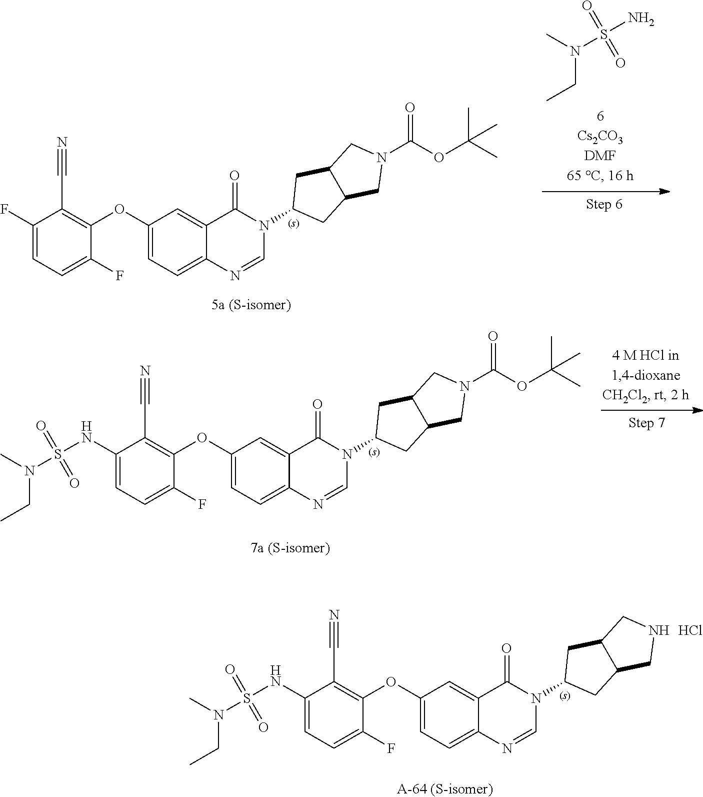

### Step 6

Into a 250 mL double-neck round-bottom flask containing a well-stirred solution of tert-butyl 5-[6-(2-cyano-3,6-difluoro-phenoxy)-4-oxo-quinazolin-3-yl]-3,3a,4,5,6,6a-hexahydro-1H-cyclopenta[c]pyrrole-2-carboxylate (5a, 5 g, 9.83 mmol) in N,N-dimethylformamide (50 mL) was added Cs₂CO₃ (9.61 g, 29.50 mmol) followed by [methyl(sulfamoyl)amino]ethane (6, 2.72 g, 19.67 mmol) at room temperature and resulting reaction mixture was stirred at 65° C. for 16 hours. After completion of the reaction, the reaction mixture was quenched with water (100 mL), solid precipitated was filtered through a celite pad and celite pad was washed with water (50 mL). The filtrate was extracted with ethyl acetate (3×100 mL) and the combined organic layer was washed with water (50 mL), saturated NaCl solution (50 mL), dried over anhydrous sodium sulfate, filtered and concentrated under reduced pressure to afford crude tert-butyl 5-[6-[2-cyano-3-[[ethyl(methyl)sulfamoyl]amino]-6-fluoro-phenoxy]-4-oxo-quinazolin-3-yl]-3,3a,4,5,6,6a-hexahydro-1H-cyclopenta[c]pyrrole-2-carboxylate (7a, 4.8 g, 6.99 mmol, 71% yield) as a brown gum. UPLC-MS (ES+): m/z 627.2 [M+H]⁺.

### Step 7

Into a 250 mL single-neck round-bottom flask containing a well-stirred solution of tert-butyl 5-[6-[2-cyano-3-[[ethyl(methyl)sulfamoyl]amino]-6-fluoro-phenoxy]-4-oxo-quinazolin-3-yl]-3,3a,4,5,6,6a-hexahydro-1H-cyclopenta[c]pyrrole-2-carboxylate (7a, 4.8 g, 7.66 mmol, 1.91 mL) in dichloromethane (50 mL) was added hydrogen chloride solution (4 M in 1,4-dioxane, 19.15 mL) at 0° C. Resulting reaction mixture was stirred at room temperature for 2 hours. After completion of the reaction, the reaction mixture was concentrated under reduced pressure to get a crude residue which was triturated with n-pentane (2×100 mL) and dried under reduced pressure to afford crude 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-64, 4 g, 6.27 mmol, 82% yield, HCl salt) as an off-white solid. UPLC-MS (ES+): m/z 527.2 [M+H]⁺.

### Synthesis A65: Synthesis of 3-[(3R)-8-azaspiro[4.5]decan-3-yl]-6-[2-cyano-3-[[ethyl(methyl)sulfamoyl]amino]-6-fluoro-phenoxy]-4-oxo-quinazoline (A-65)

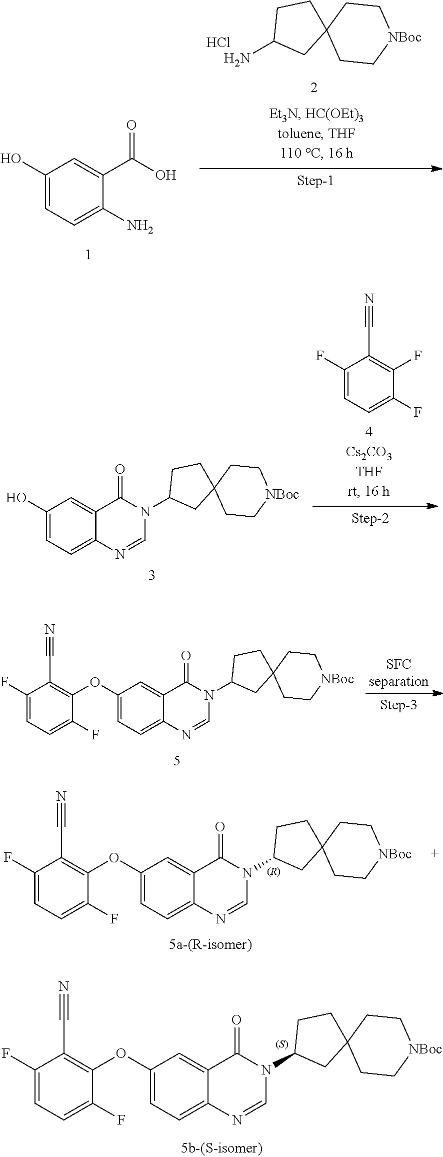

### Step 1

Into a 250 mL single-neck round-bottom flask containing a well stirred solution of tert-butyl 3-amino-8-azaspiro[4.5]decane-8-carboxylate (2, 7.31 g, 28.73 mmol, HCl salt) in toluene (60 mL) and THF (4 mL) was added TEA (78.36 mmol, 10.92 mL) and stirred at room temperature for 15 min. 2-Amino-5-hydroxy-benzoic acid (1, 4 g, 26.12 mmol) and triethyl orthoformate (39.18 mmol, 6.52 mL) were added and stirred at 110° C. for 16 hours. After completion of reaction, the reaction mixture was diluted water (200 mL) and extracted with ethyl acetate (2×100 mL). Combined organic layers were washed with 10% NaHCO₃ solution (150 mL) followed by saturated NaCl (100 mL), dried over anhydrous Na₂SO₄ filtered, filtrate was concentrated under reduced pressure to get a crude residue, which was triturated with hexane to remove non-polar impurities to afford tert-butyl 3-(6-hydroxy-4-oxo-quinazolin-3-yl)-8-azaspiro[4.5]decane-8-carboxylate (3, 6.6 g, 15.53 mmol, 59% yield) as light brown color solid. UPLC-MS (ES+): m/z 400.2 [M+H]⁺.

### Step 2

Into a 250 mL single-neck round-bottom flask containing a well-stirred solution of tert-butyl 3-(6-hydroxy-4-oxo-quinazolin-3-yl)-8-azaspiro[4.5]decane-8-carboxylate (3, 6.60 g, 16.52 mmol) in THF (90 mL) were added Cs₂CO₃ (16.15 g, 49.56 mmol) and 2,3,6-trifluorobenzonitrile (4, 19.83 mmol, 2.29 mL) and stirred at room temperature for 16 hours. After completion of reaction, the reaction mixture was diluted with water (100 mL), extracted with ethyl acetate (2×80 mL). Combined organic layers were washed with saturated NaCl solution (100 mL) dried over anhydrous Na₂SO₄, filtered and concentrated under reduced pressure to get a crude residue which was purified by flash silica-gel (100-200 mesh) column chromatography using a gradient of 0-100% ethyl acetate/hexane to afford tert-butyl 3-[6-(2-cyano-3,6-difluoro-phenoxy)-4-oxo-quinazolin-3-yl]-8-azaspiro[4.5]decane-8-carboxylate (5, 5.6 g, 9.67 mmol, 59% yield) as an off-white solid. UPLC-MS (ES+): m/z 481.2 [M+H]⁺.

Step 3: Chiral SFC separation: Above isomeric mixture tert-butyl 3-[6-(2-cyano-3,6-difluoro-phenoxy)-4-oxo-quinazolin-3-yl]-8-azaspiro[4.5]decane-8-carboxylate (5, 5.6 g, 9.67 mmol, 58.56% yield, 92.7% purity) was purified by chiral SFC following the below method.

### Method of Analysis for Chiral SFC Separation

Instrument: PIC-22-017
Column: Lux C₂(250*20)mm, 5 μm
Mobile Phase: CO₂: 0.5% IPAm in IPA (50:50)
Total Flow: 50 mL/min
Back pressure: 120 bar
Wavelength: 220 nm
Cycle time: 12.0.0 min

Fast eluting isomer (Peak-1): Fast eluting compound (Peak-1, t_{R}=3.1 min) was collected and concentrated under reduced pressure and lyophilized to afford tert-butyl(3R)-3-[6-(2-cyano-3,6-difluoro-phenoxy)-4-oxo-quinazolin-3-yl]-8-azaspiro[4.5]decane-8-carboxylate (5a, 1.2 g, 2.21 mmol, 18% yield) as an off-white solid. Peak-1 was designated as an R-isomer based on preliminary data. UPLC-MS (ES+): m/z 481.2 [M+H]⁺.

Late eluting isomer (Peak-2): Late eluting compound (Peak-2, t_{R}=4.2 min) was collected and concentrated under reduced pressure and lyophilized to afford tert-butyl (3S)-3-[6-(2-cyano-3,6-difluoro-phenoxy)-4-oxo-quinazolin-3-yl]-8-azaspiro[4.5]decane-8-carboxylate (5b, 1 g, 1.85 mmol, 15% yield) as an off-white solid. Peak-2 was designated as an S-isomer based on preliminary data. UPLC-MS (ES+): m/z 481.2 [M+H]⁺.

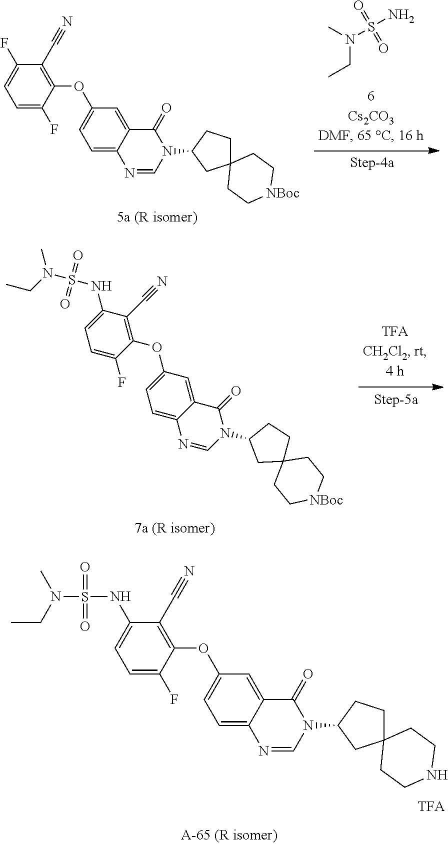

### Step 4a

Into a 100 mL single-neck round bottom flask containing a well stirred solution of tert-butyl(3R)-3-[6-(2-cyano-3,6-difluoro-phenoxy)-4-oxo-quinazolin-3-yl]-8-azaspiro[4.5]decane-8-carboxylate (5a, 1.20 g, 2.24 mmol) in anhydrous N,N-dimethylformamide (20 mL) was added Cs₂CO₃ (2.19 g, 6.71 mmol) and [methyl(sulfamoyl)amino]ethane (6, 618.10 mg, 4.47 mmol) at room temperature under nitrogen atmosphere and stirred at 65° C. for 16 hours. After completion of reaction, the reaction mixture was diluted with water (40 mL), after dilution a solid precipitated was formed. The precipitate was filtered through a Buchner funnel to remove the fluorescent impurity and the filtrate was extracted with ethyl acetate (3×50 mL). The combined organic layers were washed with cold water (2×30 mL) and dried over anhydrous Na₂SO₄, filtered, concentrated under reduced pressure to afford crude tert-butyl(3R)-3-[6-[2-cyano-3-[[ethyl(methyl)sulfamoyl]amino]-6-fluoro-phenoxy]-4-oxo-quinazolin-3-yl]-8-azaspiro[4.5]decane-8-carboxylate (7a, 1.2 g, 1.72 mmol, 77% yield) as brown gum. UPLC-MS (ES+): m/z 599.0 [M+H−t-Bu]⁺.

### Step 5a

Into a 100 mL single-neck round-bottom flask containing a well-stirred solution of tert-butyl(3R)-3-[6-[2-cyano-3-[[ethyl(methyl)sulfamoyl]amino]-6-fluoro-phenoxy]-4-oxo-quinazolin-3-yl]-8-azaspiro[4.5]decane-8-carboxylate (7a, 1.20 g, 1.83 mmol) in anhydrous dichloromethane (10 mL) was added trifluoroacetic acid (8.33 mmol, 1.40 mL) slowly at 0° C. under nitrogen atmosphere. The reaction mixture was stirred at room temperature for 4 hours. After completion of reaction, the reaction mixture was concentrated under reduced pressure to get a crude residue, which was triturated with methyl tert-butyl ether (2×10 mL) and dried under reduced pressure to afford 3-[(3R)-8-azaspiro[4.5]decan-3-yl]-6-[2-cyano-3-[[ethyl(methyl)sulfamoyl]amino]-6-fluoro-phenoxy]-4-oxo-quinazoline (A-65, 1.0 g, 1.49 mmol, 81% yield, TFA salt) as an off-white gummy liquid. UPLC-MS (ES+): m/z 555.2 [M+H]+

### Synthesis A66: Synthesis of 3-[(3S)-8-azaspiro[4.5]decan-3-yl]-6-[2-cyano-3-[[ethyl(methyl)sulfamoyl]amino]-6-fluoro-phenoxy]-4-oxo-quinazoline (A-66)

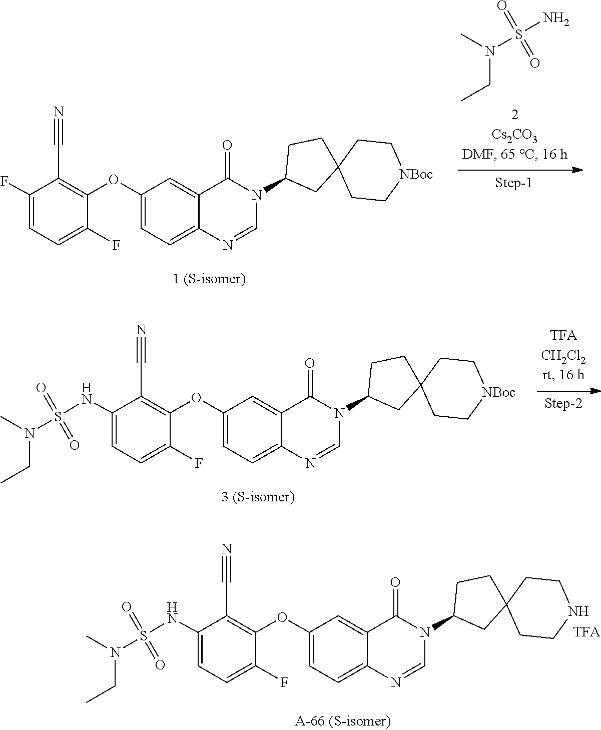

### Step 1

Into a 50 mL single-neck round-bottom flask containing a well-stirred solution of tert-butyl (3S)-3-[6-(2-cyano-3,6-difluoro-phenoxy)-4-oxo-quinazolin-3-yl]-8-azaspiro[4.5]decane-8-carboxylate (1, 1.00 g, 1.86 mmol) in anhydrous N,N-dimethylformamide (20 mL) were added Cs₂CO₃ (1.82 g, 5.59 mmol) and [methyl(sulfamoyl)amino]ethane (2, 515.08 mg, 3.73 mmol) at room temperature under nitrogen atmosphere and stirred at 65° C. for 16 hours. After completion of reaction, the reaction mixture was diluted with water (40 mL), after dilution a solid precipitated was formed. The precipitate was filtered through a Buchner funnel to remove the fluorescent impurities and the filtrate was extracted with ethyl acetate (2×50 mL). Combined organic layers were washed with cold water (2×50 mL) and dried over anhydrous Na₂SO₄, filtered and concentrated under reduced pressure to afford crude tert-butyl (3S)-3-[6-[2-cyano-3-[[ethyl(methyl)sulfamoyl]amino]-6-fluoro-phenoxy]-4-oxo-quinazolin-3-yl]-8-azaspiro[4.5]decane-8-carboxylate (3, 0.75 g, 1.14 mmol, 61% yield) as brown gummy liquid. UPLC-MS (ES+): m/z 599.3 [M+H−t-Bu]⁺.

### Step 2

Into a 50 mL single-neck round-bottom flask containing a well stirred solution of tert-butyl (3S)-3-[6-[2-cyano-3-[[ethyl(methyl)sulfamoyl]amino]-6-fluoro-phenoxy]-4-oxo-quinazolin-3-yl]-8-azaspiro[4.5]decane-8-carboxylate (3, 750.00 mg, 1.15 mmol) in anhydrous dichloromethane (15.00 mL) was added trifluoroacetic acid (11.45 mmol, 876.56 μL) slowly at 0° C. under nitrogen atmosphere. The reaction mixture was stirred at room temperature for 16 hours. After completion of reaction, the reaction mixture was concentrated under reduced pressure to get a crude residue which was triturated with methyl tert-butyl ether (2×5 mL) to remove non-polar impurities and dried under reduced pressure to afford 3-[(3S)-8-azaspiro[4.5]decan-3-yl]-6-[2-cyano-3-[[ethyl(methyl)sulfamoyl]amino]-6-fluoro-phenoxy]-4-oxo-quinazoline (A-66, 500 mg, 650.56 μmol, 57% yield, TFA salt) as a brown gummy liquid. UPLC-MS (ES+): m/z 555.4 [M+H]⁺.

### Synthesis A67: Synthesis of (3R)-3-[6-[2-cyano-3-[[ethyl(methyl)sulfamoyl]amino]-6-fluoro-phenoxy]-5-fluoro-4-oxo-quinazolin-3-yl]-1-oxa-8-azaspiro[4.5]decane (A-67)

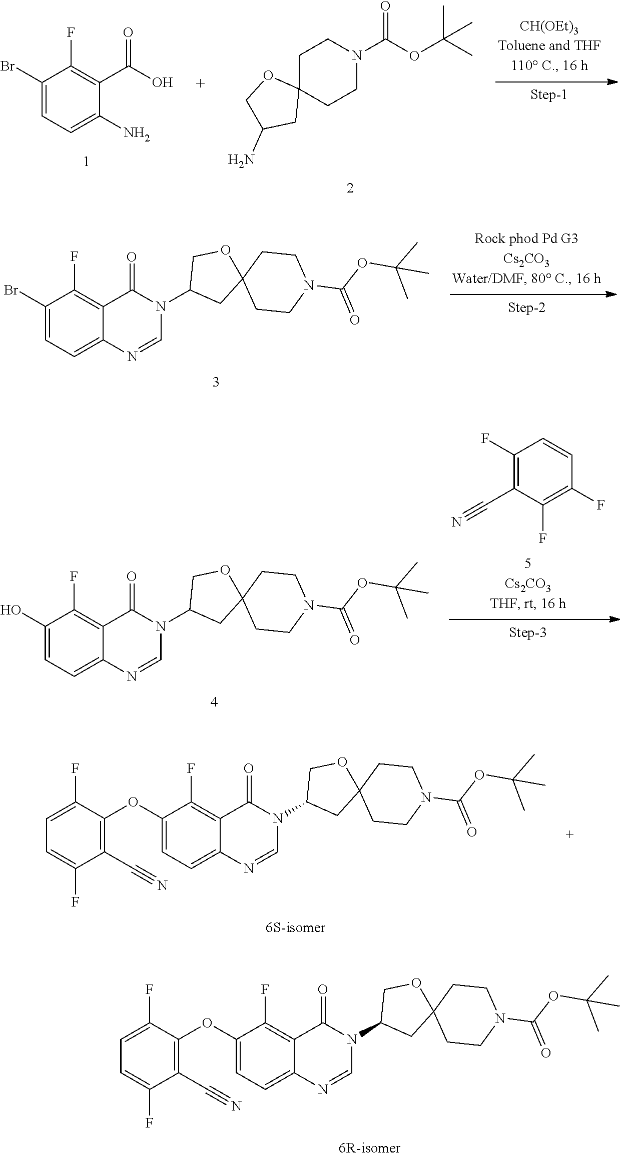

### Step 1

Into a 250 mL two neck round bottom flask containing a well stirred solution of 6-amino-3-bromo-2-fluoro-benzoic acid (1, 9.2 g, 39.31 mmol) and tert-butyl 3-amino-1-oxa-8-azaspiro[4.5]decane-8-carboxylate (2, 11.33 g, 39.31 mmol) in toluene (133.79 mL) and THF (36.49 mL) was added triethyl orthoformate (157.25 mmol, 26.16 mL) under nitrogen atmosphere at room temperature. The reaction mixture was stirred at 110° C. for 16 hours. The reaction mixture was quenched with water and extracted with ethyl acetate (3×250 mL). The combined organic layers were washed with H₂O (1×250 mL) and saturated saturated NaCl (1×250 mL), dried over Na₂SO₄, concentrated under reduced pressure to get the crude which was triturated with 5% ethyl acetate in petroleum ether to afford tert-butyl 3-(6-bromo-5-fluoro-4-oxo-quinazolin-3-yl)-1-oxa-8-azaspiro[4.5]decane-8-carboxylate (3, 4.1 g, 7.23 mmol, 18% yield) as brown solid. LC-MS(ES+): m/z 428.0 [M+H]⁺.

### Step 2

Into a 100 mL two neck round bottom flask containing a well stirred solution of tert-butyl 3-(6-bromo-5-fluoro-4-oxo-quinazolin-3-yl)-1-oxa-8-azaspiro[4.5]decane-8-carboxylate (3, 4.1 g, 7.23 mmol) in N,N-dimethylformamide (30.11 mL) and water (0.4 mL) was added Cs₂CO₃ (7.06 g, 21.68 mmol). The reaction mixture was purged with N2 gas for 10 min and added Rockphos pd G3 (605.91 mg, 722.69 mol) under nitrogen atmosphere at room temperature. The reaction mixture was stirred at 80° C. for 16 hours. The reaction mixture was quenched with water and extracted with ethyl acetate (2×250 mL). The combined organic layers were washed with H₂O (1×100 mL) and saturated NaCl (1×100 mL), dried over Na₂SO₄, concentrated under reduced pressure to get the crude tert-butyl 3-(5-fluoro-6-hydroxy-4-oxo-quinazolin-3-yl)-1-oxa-8-azaspiro[4.5]decane-8-carboxylate (4, 7.5 g, 10.52 mmol, 145% yield) as a brown solid. The crude product contains inorganic impurities, resulting in an apparent product yield over 100%. LC-MS(ESI): m/z 420.1 [M+H]⁺.

### Step 3

Into a 250 mL double neck round bottom flask containing a stirred solution of tert-butyl 3-(5-fluoro-6-hydroxy-4-oxo-quinazolin-3-yl)-1-oxa-8-azaspiro[4.5]decane-8-carboxylate (4, 7.5 g, 10.52 mmol) in THF (101.19 mL) were added 2,3,6-trifluorobenzonitrile (5, 12.62 mmol, 1.46 mL) and Cs₂CO₃ (10.28 g, 31.55 mmol) at room temperature. The reaction mixture was stirred at room temperature for 16 hours. After reaction completion, the reaction mixture was diluted with ice cold water (200 mL) and extracted with ethyl acetate (2×40 mL). Combined organic layers were dried over Na₂SO₄ and filtrates were concentrated under reduced pressure to get crude which was purified by column chromatography using 100-200 mesh silica gel with 0-20% methanol/dichloromethane as eluent to afford tert-butyl (3S)-3-[6-(2-cyano-3,6-difluoro-phenoxy)-5-fluoro-4-oxo-quinazolin-3-yl]-1-oxa-8-azaspiro[4.5]decane-8-carboxylate (1.1 g, 1.97 mmol, 19% yield) as pale brown solid.

The crude product was submitted to SFC purification.

Chiral SFC purification: Diastereomeric mixture of tert-butyl (3S)-3-[6-(2-cyano-3,6-difluoro-phenoxy)-5-fluoro-4-oxo-quinazolin-3-yl]-1-oxa-8-azaspiro[4.5]decane-8-carboxylate (1.1 g, 1.97 mmol, 19% yield) was purified by chiral SFC following below method.

### Method of Analysis

Instrument: PIC 175
Column: LUX A1-(250*20)mm, 5 μm
Mobile Phase: CO₂:IPA[65:35]
Total Flow: 60 ml/min
Back pressure: 100 bar
Wavelength: 220 nm
Cycle time: 5 min

The fractions were concentrated under reduced pressure to afford

Peak-1: tert-butyl (3S)-3-[6-(2-cyano-3,6-difluoro-phenoxy)-5-fluoro-4-oxo-quinazolin-3-yl]-1-oxa-8-azaspiro[4.5]decane-8-carboxylate (6S, 1.1 g, 1.97 mmol, 19% yield) as pale yellow solid. Peak-1 was designated as an S-isomer based on preliminary data. LC-MS (ES+): m/z 501.0 [M−^{t}butyl+H]⁺.

Peak-2: tert-butyl(3R)-3-[6-(2-cyano-3,6-difluoro-phenoxy)-5-fluoro-4-oxo-quinazolin-3-yl]-1-oxa-8-azaspiro[4.5]decane-8-carboxylate (6R, 1.3 g, 2.33 mmol, 22% yield) as white solid. Peak-2 was designated as an R-isomer based on preliminary data. LC-MS (ES+): m/z 501.0 [M−^{t}butyl+H]⁺.

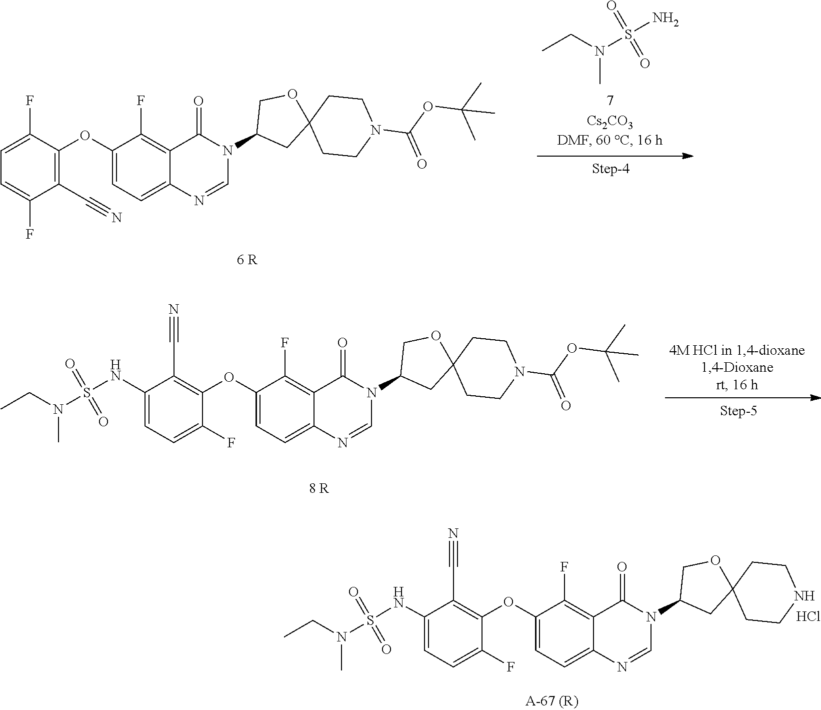

### Step 4

Into a 50 mL double neck round bottom flask containing a stirred solution of tert-butyl(3R)-3-[6-(2-cyano-3,6-difluoro-phenoxy)-5-fluoro-4-oxo-quinazolin-3-yl]-1-oxa-8-azaspiro[4.5]decane-8-carboxylate (6R, 1.3 g, 2.33 mmol) in N,N-dimethylformamide (15 mL) were added Cesium carbonate (2.28 g, 7.00 mmol) and [methyl(sulfamoyl)amino]ethane (7, 644.88 mg, 4.67 mmol) at room temperature. The reaction mixture was stirred at 60° C. for 16 hours. After completion, the reaction mixture was diluted with water (50 mL). The reaction mixture was filtered through sintered-funnel to remove the fluorescent impurity. The filtrate was extracted with ethyl acetate (3×50 mL). Combined organic layers were washed with cold water (2×500 mL), dried over sodium sulphate, filtered and concentrated under reduced pressure to afford crude tert-butyl(3R)-3-[6-[2-cyano-3-[[ethyl(methyl)sulfamoyl]amino]-6-fluoro-phenoxy]-5-fluoro-4-oxo-quinazolin-3-yl]-1-oxa-8-azaspiro[4.5]decane-8-carboxylate (8R, 1.3 g, 1.49 mmol, 64% yield) as brown gummy compound. LC-MS(ESI): m/z 619.2 [M−^{t}But+H]⁺.

### Step 5

Into a 50 mL single neck round bottom flask containing a well-stirred solution of (3R)-3-[6-[2-cyano-3-[[ethyl(methyl)sulfamoyl]amino]-6-fluoro-phenoxy]-5-fluoro-4-oxo-quinazolin-3-yl]-8-(3,3-dimethylbutanoyl)-1-oxa-8-azaspiro[4.5]decane (8R, 1.30 g, 1.57 mmol) in anhydrous 1,4-dioxane (1 mL) was added 4M HCl in 1,4 dioxane (1.57 mmol, 10 mL) at 0° C. under nitrogen atmosphere. The resulting mixture was stirred at room temperature for 16 hours. The solvents were evaporated under reduced pressure to get (3R)-3-[6-[2-cyano-3-[[ethyl(methyl)sulfamoyl]amino]-6-fluoro-phenoxy]-5-fluoro-4-oxo-quinazolin-3-yl]-1-oxa-8-azaspiro[4.5]decane (A-67, 1 g, 1.37 mmol, 87% yield, HCl salt) as colorless oil. LC-MS(ES+): m/z 575.1 [M+H]⁺.

### Synthesis A68: Synthesis of (3R)-3-[6-[2-cyano-3-[[ethyl(methyl)sulfamoyl]amino]-6-fluoro-anilino]-4-oxo-quinazolin-3-yl]-1-oxa-8-azaspiro[4.5]decane (A-68)

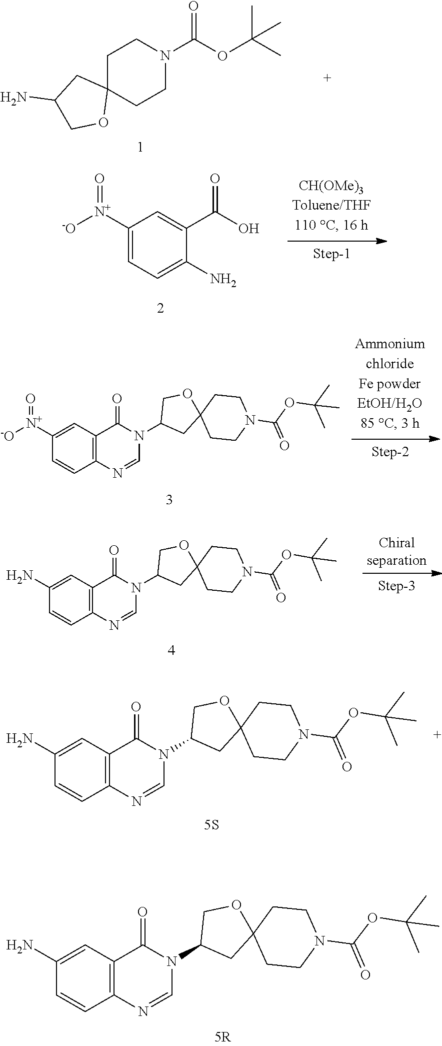

### Step 1

Into 250 mL double neck round bottom flask containing a stirred solution of tert-butyl 3-amino-1-oxa-8-azaspiro[4.5]decane-8-carboxylate (1, 10 g, 39.01 mmol) in toluene (50 mL) and THF (50 mL) were added 2-amino-5-nitro-benzoic acid (2, 7.11 g, 39.01 mmol) and trimethyl orthoformate (117.03 mmol, 12.83 mL) at ambient temperature under nitrogen atmosphere. The resulting mixture was stirred at 110° C. for 16 hours. The reaction mixture was cooled to room temperature and poured into water (250 mL) and extracted with ethyl acetate (3×250 mL). The combined organic layers were washed with NaHCO₃ (500 mL) and dried over anhydrous Na₂SO₄, filtered and concentrated under reduced pressure to give a crude residue. The crude residue was purified by flash silica-gel (230-400 mesh) column with 0-100% ethyl acetate/hexane to afford tert-butyl 3-(6-nitro-4-oxo-quinazolin-3-yl)-1-oxa-8-azaspiro[4.5]decane-8-carboxylate (3, 7.0 g, 15.32 mmol, 39% yield) as a pale brown solid. UPLC-MS (ES+): m/z 375.1 [M−^{t}Bu+H]⁺.

### Step 2

Into a 250 mL double neck round bottom flask containing a stirred solution of tert-butyl 3-(6-nitro-4-oxo-quinazolin-3-yl)-1-oxa-8-azaspiro[4.5]decane-8-carboxylate (3, 7.00 g, 16.26 mmol) in water (35 mL) and ethanol (35 mL) were added Iron powder (9.08 g, 162.62 mmol, 1.16 mL) and ammonium chloride (8.70 g, 162.62 mmol, 5.69 mL) at room temperature. The reaction mixture was stirred at 85° C. for 3 hours. After completion of the reaction, the reaction mixture was filtered and concentrated under reduced pressure to get crude residue. The crude was dissolved in water (50 mL), extracted with ethyl acetate (3×100 mL). The combined organic layers were dried with anhydrous Na₂SO₄, filtered and the filtrate was evaporated under reduced pressure to get crude product which was purified by column chromatography (100 g, 100-200 mesh) using a gradient of 0-100% ethyl acetate/hexane, while the desired product eluted at 30-40% ethyl acetate/hexane to afford tert-butyl 3-(6-amino-4-oxo-quinazolin-3-yl)-1-oxa-8-azaspiro[4.5]decane-8-carboxylate (4, 6.0 g, 14.48 mmol, 89% yield) as yellow solid. LC-MS(ES+): m/z 401.2 [M+H]⁺.

Step 3: Isomers of Step 2 (6 g) was separated by SFC following below method
Instrument: PIC 22-016
Column: I Cellulose Z-(250*30)mm, 5 μm
Mobile Phase: CO₂: 0.5% IPAm in methanol [60:40]
Total Flow: 100 mL/min
Back pressure: 100 bar
Wavelength: 220 nm
Cycle time: 5 min

The fractions were concentrated under reduced pressure to afford

Peak-1: tert-butyl (3S)-3-(6-amino-4-oxo-quinazolin-3-yl)-1-oxa-8-azaspiro[4.5]decane-8-carboxylate (5S, 2.6 g, 6.07 mmol, 40% yield) as a pale yellow solid. LC-MS(ESI): m/z 401.2 [M+H]⁺. Peak-1 was designated as an S-isomer based on preliminary data.

Peak-2: tert-butyl(3R)-3-(6-amino-4-oxo-quinazolin-3-yl)-1-oxa-8-azaspiro[4.5]decane-8-carboxylate (5R, 2.2 g, 5.25 mmol, 35% yield) as a pale yellow solid. LC-MS(ESI): m/z 401.2 [M+H]⁺. Peak-2 was designated as an R-isomer based on preliminary data.

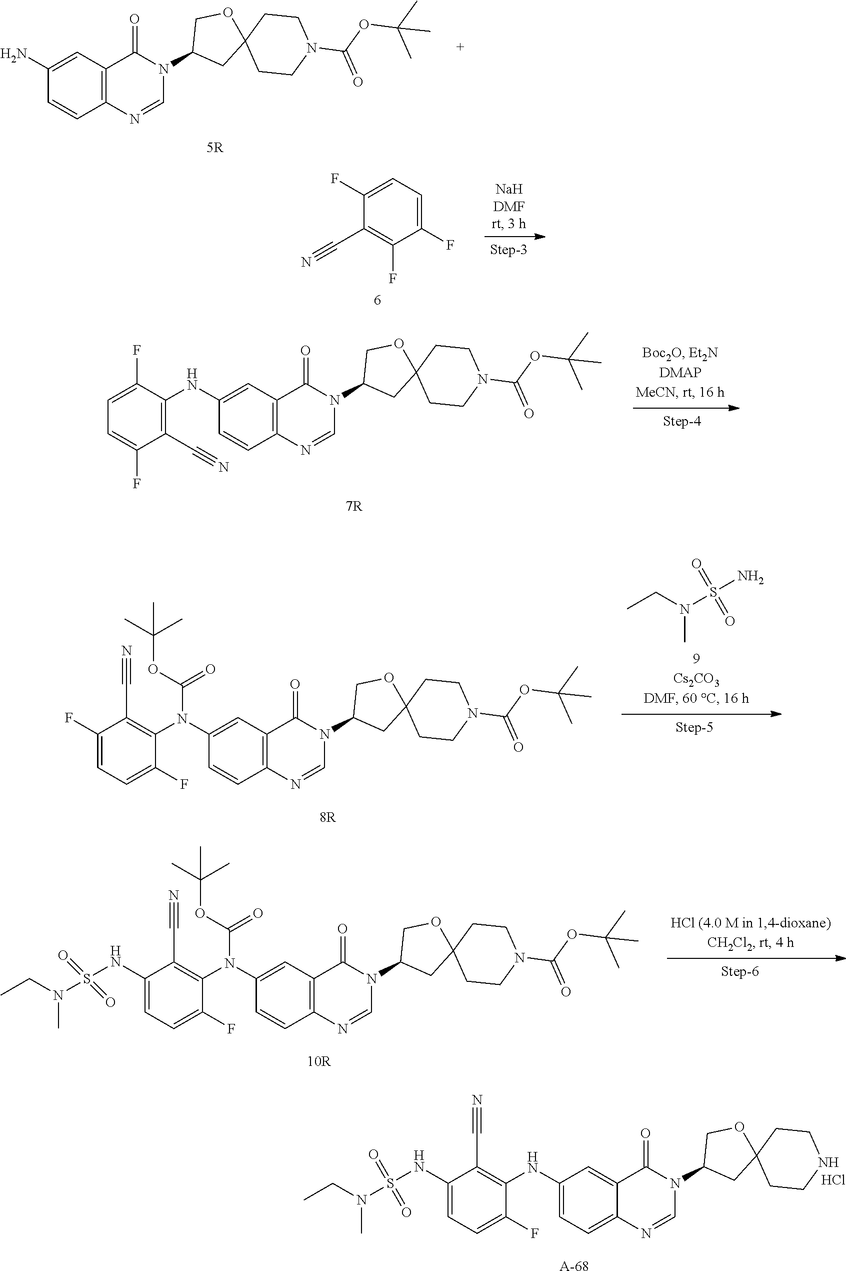

### Step 3

Into a 100 mL two-neck round-bottom flask containing a well-stirred solution of Sodium Hydride (in oil dispersion) 60% dispersion in mineral oil (682.30 mg, 17.06 mmol, 60% purity) and tert-butyl(3R)-3-(6-amino-4-oxo-quinazolin-3-yl)-1-oxa-8-azaspiro[4.5]decane-8-carboxylate (5R, 2.30 g, 5.69 mmol) in anhydrous N,N-dimethylformamide (30 mL) at 0° C. and the resulting reaction mixture was stirred at 0° C. for 30 minutes followed by addition of 2,3,6-trifluorobenzonitrile (6, 11.37 mmol, 1.31 mL) at ambient temperature under nitrogen atmosphere. The reaction mixture was stirred at room temperature for 3 hours. The reaction mixture was quenched by adding ice-cold water and extracted with ethyl acetate (3×100 mL) and dried over sodium sulphate to get crude residue which was purified by flash silica-gel (100-200 mesh) column with 0-100% ethyl acetate/hexane to afford tert-butyl(3R)-3-[6-(2-cyano-3,6-difluoro-anilino)-4-oxo-quinazolin-3-yl]-1-oxa-8-azaspiro[4.5]decane-8-carboxylate (7R, 3.0 g, 3.68 mmol, 65% yield) as an off-white solid. LC-MS(ESI): m/z 538.2 [M+H]⁺.

### Step 4

Into a 250 mL multi neck round bottom flask containing a well-stirred solution of tert-butyl 3-[(3R)-6-(2-cyano-3,6-difluoro-anilino)-4-oxo-quinazolin-3-yl]-1-oxa-8-azaspiro[4.5]decane-8-carboxylate (7R, 3.00 g, 3.68 mmol) in anhydrous acetonitrile (50 mL) was added triethylamine (11.05 mmol, 1.54 mL) at 0-5° C., followed by the addition of Di-tert-butyl dicarbonate (7.37 mmol, 1.69 mL) and DMAP (225.00 mg, 1.84 mmol) at same temperature. The resulting mixture was stirred at room temperature for 16 hours. The reaction mixture was concentrated under reduced pressure to get crude residue. Crude residue was dissolved in ethyl acetate (200 mL) and washed with water (200 mL). The combined organic layers were dried over anhydrous Na₂SO₄, filtered and concentrated under reduced pressure to get crude residue which was purified by flash silica-gel (100-200 mesh) column with 0-100% ethyl acetate/hexane to afford tert-butyl(3R)-3-[6-(N-tert-butoxycarbonyl-2-cyano-3,6-difluoro-anilino)-4-oxo-quinazolin-3-yl]-1-oxa-8-azaspiro[4.5]decane-8-carboxylate (8R, 1.6 g, 2.46 mmol, 67% yield) as white solid. LC-MS(ESI): m/z 638.4 [M+H]⁺.

### Step 5

Into a 40 mL pressure vial containing a stirred solution of tert-butyl 3-[(3R)-6-(N-tert-butoxycarbonyl-2-cyano-3,6-difluoro-anilino)-4-oxo-quinazolin-3-yl]-1-oxa-8-azaspiro[4.5]decane-8-carboxylate (8R, 1.02 g, 1.57 mmol) in N,N-dimethylformamide (10 mL) were added cesium carbonate (1.53 g, 4.70 mmol) and [methyl(sulfamoyl)amino]ethane (9, 325.06 mg, 2.35 mmol) at room temperature. The reaction mixture was stirred at 60° C. for 16 hours. After completion, the reaction mixture was diluted with water (50 mL) and filtered through sintered funnel to remove the fluorescent impurity. The filtrate was extracted with ethyl acetate (3×50 mL). Combined organic layers were washed with cold water (2×100 mL), dried over sodium sulphate, filtered and concentrated under reduced pressure to get crude which was purified by flash silica-gel (60-120 mesh) column with 0-100% ethyl acetate/petroleum ether to afford tert-butyl 3-[(3R)-6-[N-tert-butoxycarbonyl-2-cyano-3-[[ethyl(methyl)sulfamoyl]amino]-6-fluoro-anilino]-4-oxo-quinazolin-3-yl]-1-oxa-8-azaspiro[4.5]decane-8-carboxylate (10R, 0.4 g, 428.65 mol, 27% yield) as off-white solid. LC-MS(ESI): m/z 756.2 [M+H]⁺.

### Step 6

Into a 25 mL single neck round bottom flask containing a stirred solution of tert-butyl(3R)-3-[6-[N-tert-butoxycarbonyl-2-cyano-3-[[ethyl(methyl)sulfamoyl]amino]-6-fluoro-anilino]-4-oxo-quinazolin-3-yl]-1-oxa-8-azaspiro[4.5]decane-8-carboxylate (10R, 400.00 mg, 529.20 mol) in DCM (10 mL) was added HCl (4.0 M in 1,4-dioxane) (4 M, 264.60 L) at 0° C. The reaction mixture was stirred at room temperature for 4 hours. After completion, solvents were evaporated under reduced pressure and triturated with methyl tert-butyl ether (20 mL) to afford (3R)-3-[6-[2-cyano-3-[[ethyl(methyl)sulfamoyl]amino]-6-fluoro-anilino]-4-oxo-quinazolin-3-yl]-1-oxa-8-azaspiro[4.5]decane (A-68, 300 mg, 408.39 mol, 77% yield, HCl salt) as a yellow solid. UPLC-MS (ES+): m/z 556.2 [M+H]⁺.

### Synthesis A69: Synthesis of (3R)-3-[6-[2-cyano-3-[[ethyl(methyl)sulfamoyl]amino]-6-fluoro-anilino]-5-fluoro-4-oxo-quinazolin-3-yl]-1-oxa-8-azaspiro[4.5]decane (A-69)

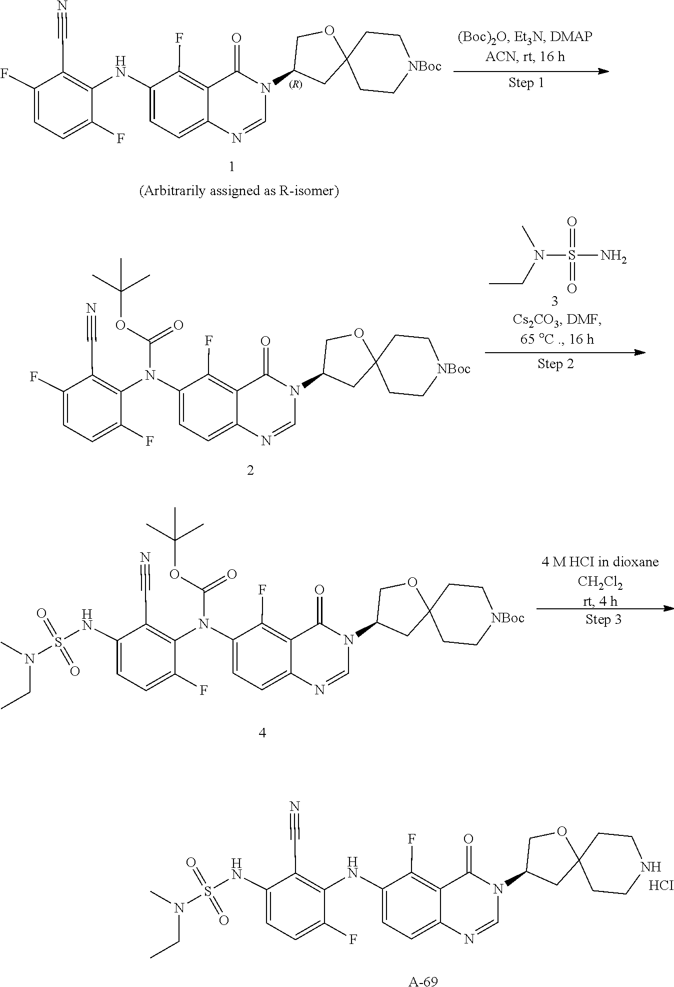

### Step 1

Into a 50 mL single-neck round bottom flask containing a well-stirred solution of tert-butyl(3R)-3-[6-(2-cyano-3,6-difluoro-anilino)-5-fluoro-4-oxo-quinazolin-3-yl]-1-oxa-8-azaspiro[4.5]decane-8-carboxylate (1, 427 mg, 760.93 μmol) in anhydrous acetonitrile (5.01 mL) was added DMAP (92.96 mg, 760.93 μmol) and triethylamine (3.80 mmol, 530.29 μL) followed by di-tert-butyl dicarbonate (2.28 mmol, 523.89 μL) was added at room temperature under nitrogen atmosphere. The reaction mixture was stirred at room temperature for 16 hours. After completion of the reaction, the reaction mixture was diluted with water (50 mL) and extracted with ethyl acetate (3×20 mL). Combined organic layers were washed with cold water (40 mL), dried over anhydrous Na₂SO₄, filtered and concentrated under reduced pressure to get a crude residue which was purified by flash silica-gel (230-400 mesh, 25 g) column chromatography using a gradient of 0-100% ethyl acetate/hexane to afford tert-butyl(3R)-3-[6-(N-tert-butoxycarbonyl-2-cyano-3,6-difluoro-anilino)-5-fluoro-4-oxo-quinazolin-3-yl]-1-oxa-8-azaspiro[4.5]decane-8-carboxylate (2, 480 mg, 644.23 μmol, 85% yield) as a brown solid. UPLC-MS (ES+): m/z 656.3 [M+H]⁺.

### Step 2

Into a 50 mL three-neck round bottom flask containing a well-stirred solution of tert-butyl(3R)-3-[6-(N-tert-butoxycarbonyl-2-cyano-3,6-difluoro-anilino)-5-fluoro-4-oxo-quinazolin-3-yl]-1-oxa-8-azaspiro[4.5]decane-8-carboxylate (2, 480 mg, 732.08 mol) in anhydrous N,N-dimethylformamide (5.5 mL) was added [methyl(sulfamoyl)amino]ethane (3, 303.50 mg, 2.20 mmol) and Cs₂CO₃ (954.11 mg, 2.93 mmol) at room temperature under nitrogen atmosphere. The reaction mixture was stirred at 65° C. for 16 hours. After completion of the reaction, the reaction mixture was cooled to room temperature and diluted with water (20 mL) and stirred for 10 min. The precipitated solid was filtered, and filtrate was diluted with water (30 mL) and extracted with ethyl acetate (2×100 mL). The combined organic layer was washed with saturated NaCl (10 mL), dried under anhydrous sodium sulphate, filtered and concentrated under reduced pressure to afford tert-butyl(3R)-3-[6-[N-tert-butoxycarbonyl-2-cyano-3-[[ethyl(methyl)sulfamoyl]amino]-6-fluoro-anilino]-5-fluoro-4-oxo-quinazolin-3-yl]-1-oxa-8-azaspiro[4.5]decane-8-carboxylate (4, 237 mg, 245.93 mol, 34% yield) as a brown gummy liquid. UPLC-MS (ES+): m/z 772.2 [M+H]⁺.

### Step 3

Into a 50 mL single-neck round-bottom flask containing a well-stirred solution of tert-butyl(3R)-3-[6-[N-tert-butoxycarbonyl-2-cyano-3-[[ethyl(methyl)sulfamoyl]amino]-6-fluoro-anilino]-5-fluoro-4-oxo-quinazolin-3-yl]-1-oxa-8-azaspiro[4.5]decane-8-carboxylate (4, 237 mg, 245.01 mol) in anhydrous 1,4-dioxane (3 mL) was added hydrogen chloride solution (4.0 M in 1,4-dioxane, 306.26 μL) at 0° C. and the mixture was stirred at room temperature for 4 hours. After completion of the reaction, solvents were removed under reduced pressure to get a crude residue, which was triturated with methyl tert-butyl ether (2×2 mL) and dried under vacuum to afford (3R)-3-[6-[2-cyano-3-[[ethyl(methyl)sulfamoyl]amino]-6-fluoro-anilino]-5-fluoro-4-oxo-quinazolin-3-yl]-1-oxa-8-azaspiro[4.5]decane (A-69, 203 mg, 222.94 mol, 91% yield HCl salt) as a pale brown gummy solid. UPLC-MS (ESI): m/z 574.2 [M+H]⁺.

### Synthesis A70: Synthesis of (3S)-3-[6-[2-cyano-3-[[ethyl(methyl)sulfamoyl]amino]-6-fluoro-anilino]-5-fluoro-4-oxo-quinazolin-3-yl]-1-oxa-8-azaspiro[4.5]decane (A-70)

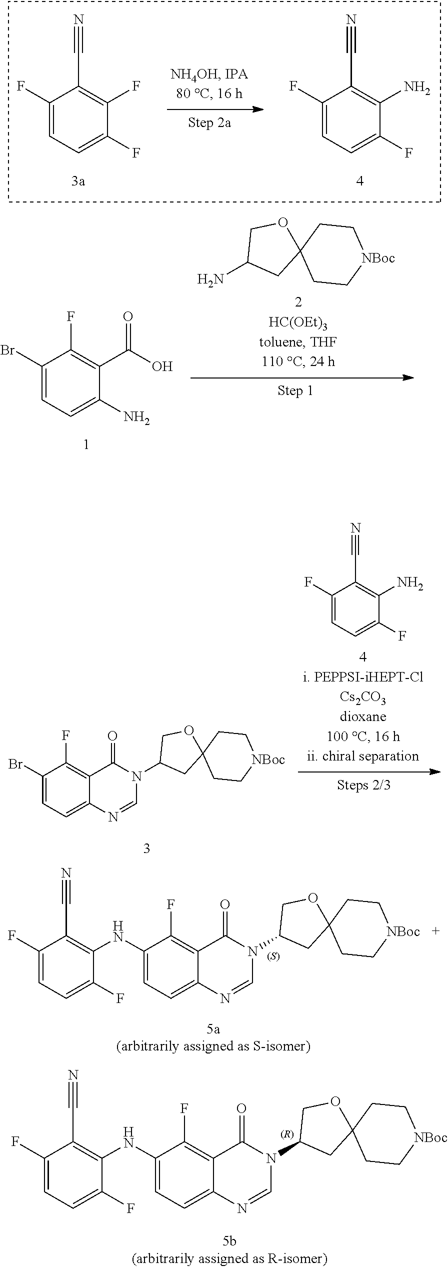

### Step 2a

Into a 100 mL pressure vial containing a solution of 2,3,6-trifluorobenzonitrile (3a, 31.83 mmol, 3.68 mL) in isopropanol (40 mL) was added ammonium;hydroxide (381.94 mmol, 20 mL, 28% purity) and the reaction mixture was stirred at 80° C. for 16 hours. After completion of the reaction, the reaction mixture was diluted with water (50 mL) and extracted with ethyl acetate (2×100 mL). Combined organic layers were washed with NaHCO₃ solution (100 mL), saturated NaCl (100 mL), dried over anhydrous Na₂SO₄ and concentrated under reduced pressure to get a crude residue which was purified by flash silica-gel (100-200 mesh) column chromatography using a gradient of 0-50% ethyl acetate/hexane to afford 2-amino-3,6-difluoro-benzonitrile (4, 4 g, 25.65 mmol, 81% yield) as an off-white solid. GC-MS: m/z 154.1 [M+H]⁺.

### Step 1

Into a 1000 mL two-neck round bottom flask containing a stirred solution of 6-amino-3-bromo-2-fluoro-benzoic acid (1, 10 g, 42.73 mmol) and tert-butyl 3-amino-1-oxa-8-azaspiro[4.5]decane-8-carboxylate (2, 10.95 g, 42.73 mmol) in anhydrous toluene (100 mL) and anhydrous THF (35 mL) was added triethyl orthoformate (170.92 mmol, 28.43 mL) to the reaction mixture and stirred at 110° C. under nitrogen atmosphere for 24 hours. After completion of the reaction, the reaction mixture was diluted with water (500 mL) and extracted with ethyl acetate (2×750 mL). Combined organic layers were washed with NaHCO₃ solution (200 mL) and saturated NaCl (200 mL), dried over anhydrous Na₂SO₄, filtered and concentrated under reduced pressure to get a crude residue which was triturated with 5% ethyl acetate/hexane to afford tert-butyl 3-(6-bromo-5-fluoro-4-oxo-quinazolin-3-yl)-1-oxa-8-azaspiro[4.5]decane-8-carboxylate (3, 10 g, 19.70 mmol, 46% yield) as a brown solid. UPLC-MS (ES+): m/z 428.1 [M−^{t-}Bu]⁺.

### Step 2/3

Into a 500 mL two neck round bottom flask containing a solution of tert-butyl 3-(6-bromo-5-fluoro-4-oxo-quinazolin-3-yl)-1-oxa-8-azaspiro[4.5]decane-8-carboxylate (3, 4 g, 8.29 mmol) and 2-amino-3,6-difluoro-benzonitrile (4, 1.92 g, 12.44 mmol) in anhydrous 1,4-dioxane (50 mL) was added Cs₂CO₃ (8.11 g, 24.88 mmol) at room temperature under nitrogen atmosphere. The reaction mixture was purged with nitrogen gas for 5-10 min and added Pd-PEPPSI-IHeptCl. Again, the reaction mixture was purged with nitrogen gas for five minutes and stirred at 100° C. for 16 h under nitrogen atmosphere. After completion of reaction, the reaction mixture was diluted with water (30 mL), extracted with ethyl acetate (2×200 mL). The organic layer was dried over anhydrous Na₂SO₄ and concentrated under reduced pressure to get a crude residue, which was purified by column chromatography (100-200 mesh) using a gradient of 0-100% ethyl acetate/hexane, while eluted with 70% ethyl acetate/hexane to afford tert-butyl 3-[6-(2-cyano-3,6-difluoro-anilino)-5-fluoro-4-oxo-quinazolin-3-yl]-1-oxa-8-azaspiro[4.5]decane-8-carboxylate (5, 1.4 g, 2.40 mmol, 28.91% yield, 95.15% purity) as pale yellow fluffy solid. Above isomeric mixture of tert-butyl 3-[6-(2-cyano-3,6-difluoro-anilino)-5-fluoro-4-oxo-quinazolin-3-yl]-1-oxa-8-azaspiro[4.5]decane-8-carboxylate (5, 1.4 g, 2.40 mmol, 29% yield) was purified by chiral SFC following the below method.

### Method of Analysis for chiral SFC separation

Instrument: PIC-22-016
Column: LUXA1-(250*20) mm, 5 m
Mobile Phase: CO₂: 0.5% ipa in IPA (65:35)
Total Flow: 60 mL/m
Back pressure: 100 bar
Wavelength: 254 nm
Cycle time: 7 min

Fast eluting isomer (Peak-1): Fast eluting compound (Peak-1) was collected and concentrated under reduced pressure to afford tert-butyl 3-[(3S)-6-(2-cyano-3,6-difluoro-anilino)-5-fluoro-4-oxo-quinazolin-3-yl]-1-oxa-8-azaspiro[4.5]decane-8-carboxylate (5a, 550 mg, 974.17 μmol, 39% yield) as a pale yellow solid. Peak-1 was designated as an S-isomer based on preliminary data. UPLC-MS (ES+): m/z 500.0 [M−^{t}Bu]⁺.

Late eluting isomer (Peak-2): Late eluting compound (Peak-2, t_{R}=4.60 min) was collected and concentrated under reduced pressure to afford tert-butyl 3-[(3R)-6-(2-cyano-3,6-difluoro-anilino)-5-fluoro-4-oxo-quinazolin-3-yl]-1-oxa-8-azaspiro[4.5]decane-8-carboxylate (5b, 600 mg, 1.08 mmol, 43% yield) as a pale yellow solid. Peak-2 was designated as an R-isomer based on preliminary data. UPLC-MS (ES+): m/z 500.0 [M−^{t}Bu]⁺.

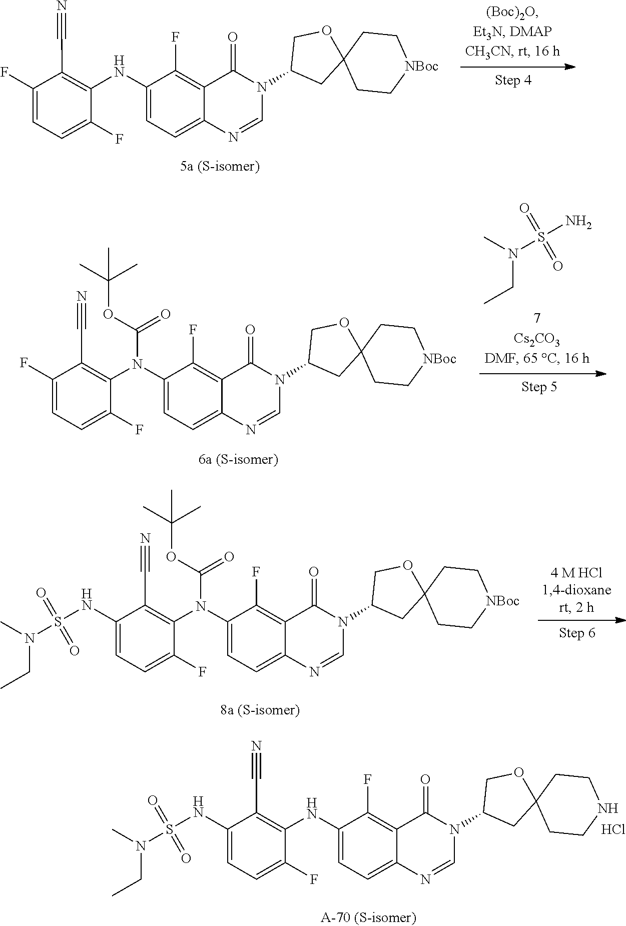

### Step 4

Into a 50 mL two-neck round bottom flask containing a stirred solution of tert-butyl (3S)-3-[6-(2-cyano-3,6-difluoro-anilino)-5-fluoro-4-oxo-quinazolin-3-yl]-1-oxa-8-azaspiro[4.5]decane-8-carboxylate (5a, 520 mg, 936.01 μmol) in anhydrous acetonitrile (6.52 mL) were added TEA (4.68 mmol, 652.31 μL) and DMAP (114.35 mg, 936.01 μmol) followed by di-tert-butyl dicarbonate (2.81 mmol, 644.43 μL) at room temperature under nitrogen atmosphere. The reaction mixture was stirred at room temperature for 16 h under nitrogen atmosphere. After completion of the reaction, the reaction mixture was diluted with water (10 mL) and extracted with ethyl acetate (3×30 mL). Combined organic layers were washed with saturated NaCl solution (30 mL) and 1.5 N HCl (30 mL), dried over anhydrous Na₂SO₄, filtered and concentrated under reduced pressure to afford crude tert-butyl (3S)-3-[6-(N-tert-butoxycarbonyl-2-cyano-3,6-difluoro-anilino)-5-fluoro-4-oxo-quinazolin-3-yl]-1-oxa-8-azaspiro[4.5]decane-8-carboxylate (6a, 600 mg, 820.57 μmol, 88% yield) as a pale gummy liquid. UPLC-MS (ES+): m/z 658.0 [M+H]⁺.

### Step 5

Into a 100 mL two neck round bottom flask containing a stirred solution of tert-butyl (3S)-3-[6-(N-tert-butoxycarbonyl-2-cyano-3,6-difluoro-anilino)-5-fluoro-4-oxo-quinazolin-3-yl]-1-oxa-8-azaspiro[4.5]decane-8-carboxylate (6a, 600 mg, 814.44 Pmol) in anhydrous N,N-dimethylformamide (10 mL) were added Cs₂CO₃ (796.09 mg, 2.44 mmol) and [methyl(sulfamoyl)amino]ethane (7, 281.37 mg, 2.04 mmol) at room temperature under nitrogen atmosphere. The reaction mixture was stirred at 65° C. for 16 h under nitrogen atmosphere. After completion of the reaction, the reaction mixture was diluted with water (20 mL) and extracted with ethyl acetate (3×20 mL). Combined organic layers were dried over anhydrous Na₂SO₄ and concentrated under reduced pressure to afford a crude residue, which was purified by column chromatography (100-200 mesh) using a gradient of 0-100% ethyl acetate/hexane to afford tert-butyl (3S)-3-[6-[N-tert-butoxycarbonyl-2-cyano-3-[[ethyl(methyl)sulfamoyl]amino]-6-fluoro-anilino]-5-fluoro-4-oxo-quinazolin-3-yl]-1-oxa-8-azaspiro[4.5]decane-8-carboxylate (8a, 320 mg, 359.10 μmol, 44% yield) as a brown gummy liquid. UPLC-MS (ES−): m/z 772.2 [M−H]⁻.

### Step 6

Into a 25 mL single-neck round bottom flask containing a well stirred solution of tert-butyl (3S)-3-[6-[N-tert-butoxycarbonyl-2-cyano-3-[[ethyl(methyl)sulfamoyl]amino]-6-fluoro-anilino]-5-fluoro-4-oxo-quinazolin-3-yl]-1-oxa-8-azaspiro[4.5]decane-8-carboxylate (8a, 320 mg, 355.63 μmol) in anhydrous 1,4-dioxane (5 mL) was added hydrogen chloride solution 4.0 M in 1,4-dioxane (4 M, 889.07 μL) slowly at 0° C. under nitrogen atmosphere. The resulting mixture was stirred at room temperature for 2 hours. After completion of the reaction, the reaction mixture was concentrated under reduced pressure to get a crude residue which was triturated with methyl tert-butyl ether (2×30 mL) and dried under reduced pressure to afford (3S)-3-[6-[2-cyano-3-[[ethyl(methyl)sulfamoyl]amino]-6-fluoro-anilino]-5-fluoro-4-oxo-quinazolin-3-yl]-1-oxa-8-azaspiro[4.5]decane (A-70, 250 mg, 352.70 μmol, 99% yield, HCl salt) as a brown gummy solid. UPLC-MS (ES+): m/z 574.2 [M+H]⁺.

### Synthesis A71: Synthesis of 3-(7-azaspiro[3.5]nonan-2-yl)-6-[2-cyano-6-fluoro-3-[[2-hydroxyethyl(methyl)sulfamoyl]amino]phenoxy]-4-oxo-quinazoline (A-71)

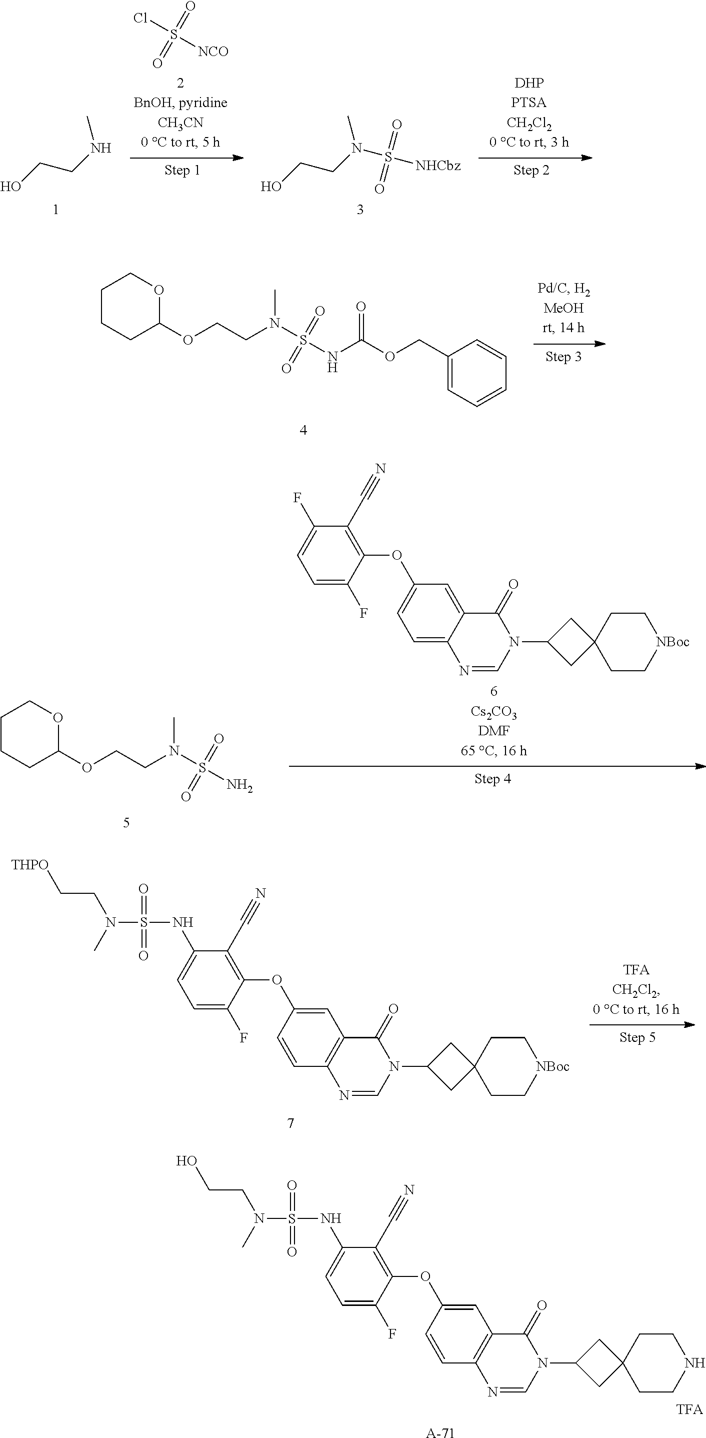

### Step 1

Into a 1 L three-neck round bottom flask containing a well stirred solution of N-(oxomethylene)sulfamoyl chloride (2, 9.42 g, 66.58 mmol, 5.80 mL) in acetonitrile (400 mL) was added benzyl alcohol (55.48 mmol, 5.74 mL) at 0° C. and resulting mixture was stirred at the same temperature for 30 min. To the above mixture, pyridine (66.58 mmol, 5.36 mL) and 2-(methylamino)ethanol (1, 221.94 mmol, 17.75 mL) were added at 0° C. and stirred for 5 h at room temperature. After completion of the reaction, the reaction mixture was quenched with 1M HCl solution and extracted with ethyl acetate (3×100 mL). Combined organic layer was washed with saturated saturated NaCl solution, dried over anhydrous Na₂SO₄, filtered and concentrated under reduced pressure to afford crude benzyl N-[2-hydroxyethyl(methyl)sulfamoyl]carbamate (3, 14 g, 48.33 mmol, 87% yield) as a colorless thick liquid. UPLC-MS (ES+): m/z 289.00 [M+H]⁺.

### Step 2

Into a 1 L three-neck round bottom flask containing a well stirred solution of benzyl N-[2-hydroxyethyl(methyl)sulfamoyl]carbamate (3, 10 g, 34.68 mmol) in dichloromethane (300.02 mL) were added p-toluenesulfonic acid monohydrate (659.75 mg, 3.47 mmol) and 3,4-dihydro-2H-pyran (52.03 mmol, 4.73 mL) at 0° C. Resulting reaction mixture was stirred at room temperature for 3 hours. After completion of the reaction, the reaction mixture was diluted with water (200 mL) and extracted with dichloromethane (3×150 mL). Combined organic layer was washed with saturated saturated NaCl solution, dried over anhydrous Na₂SO₄, filtered and concentrated under reduced pressure to afford a crude benzyl N-[methyl(2-tetrahydropyran-2-yloxyethyl)sulfamoyl]carbamate (4, 7 g, 18.79 mmol, 54% yield) as a pale yellow oil. UPLC-MS (ES−): m/z 371.20 [M−H]⁻.

### Step 3

Into a 250 mL single-neck round bottom flask containing a well stirred solution of benzyl N-[methyl(2-tetrahydropyran-2-yloxyethyl)sulfamoyl]carbamate (4, 5.80 g, 15.57 mmol) in methanol (120 mL) at room temperature and resulting mixture was degassed by bubbling nitrogen gas for 10 min. Subsequently, palladium (10% on carbon, wetted with ca. 55% Water) (0.6 g, 5.64 mmol) was added and the resulting reaction mixture was stirred at room temperature under hydrogen bladder pressure for 14 hours. After completing of the reaction, the reaction mixture was filtered through a Celite pad and Celite pad was washed with methanol (150 mL) and mixture of ethyl acetate/dichloromethane (1:1) (200 mL). Combined filtrate was concentrated under reduced pressure to afford a crude 2-[2-[methyl(sulfamoyl)amino]ethoxy]tetrahydropyran (5, 3.5 g, 14.69 mmol, 94% yield). UPLC-MS (ES−): m/z 237.2 [M−H]⁻. ¹H NMR (400 MHz, DMSO-d₆): δ 6.69 (s, 2H), 4.59 (t, J=2.4 Hz, 1H), 3.77-3.72 (m, 2H), 3.55-3.51 (m, 1H), 3.45-3.42 (m, 1H), 3.14-3.10 (m, 2H), 2.70 (s, 3H), 1.69-1.62 (m, 2H), 1.51-1.44 (m, 4H).

### Step 4

Into a 100 mL two-neck round-bottom flask containing a well stirred solution of tert-butyl 2-[6-(2-cyano-3,6-difluoro-phenoxy)-4-oxo-quinazolin-3-yl]-7-azaspiro[3.5]nonane-7-carboxylate (6, 800 mg, 1.49 mmol) and 2-[2-[methyl(sulfamoyl)amino]ethoxy]tetrahydropyran (5, 530.84 mg, 2.23 mmol) in N,N-dimethylformamide (10 mL) was added Cs₂CO₃ (1.45 g, 4.46 mmol) at room temperature. Resulting reaction mixture was stirred at 65° C. for 16 hours. After completion of the reaction, the reaction mixture was quenched with water (50 mL), precipitated solid was filtered through a Celite pad and Celite pad was washed with water (30 mL). Filtrate was extracted with ethyl acetate (3×100 mL) and combined organic layer was washed with water (50 mL), saturated NaCl solution (50 mL), dried over anhydrous Na₂SO₄, filtered and concentrated under reduced pressure to afford a crude tert-butyl-2-[6-[2-cyano-6-fluoro-3-[[methyl(2-tetrahydropyran-2-yloxyethyl)sulfamoyl]amino]phenoxy]-4-oxo-quinazolin-3-yl]-7-azaspiro[3.5]nonane-7-carboxylate (7, 800 mg, 766.70 mol, 52% yield) as a pale brown gum. UPLC-MS (ES+): m/z 739.2 [M−H]⁻.

### Step 5

Into a 100 mL single-neck round-bottom flask containing a well-stirred solution of tert-butyl 2-[6-[2-cyano-6-fluoro-3-[[methyl(2-tetrahydropyran-2-yloxyethyl)sulfamoyl]amino]phenoxy]-4-oxo-quinazolin-3-yl]-7-azaspiro[3.5]nonane-7-carboxylate (7, 0.63 g, 850.39 mol) in dichloromethane (8 mL) was added trifluoroacetic acid (4.25 mmol, 325.37 L) at 0° C. Resulting reaction mixture was stirred at room temperature for 16 hours. After completion of the reaction, the reaction mixture was concentrated under reduced pressure to afford a crude residue which was triturated with methyl tert-butyl ether (80 mL) and dried under vacuum to afford 3-(7-azaspiro[3.5]nonan-2-yl)-6-[2-cyano-6-fluoro-3-[[2-hydroxyethyl(methyl)sulfamoyl]amino]phenoxy]-4-oxo-quinazoline (A-71, 450 mg, 331.88 mol, 39% yield, TFA salt) as a colorless thick liquid. UPLC-MS (ES+): m/z 557.20 [M+H]⁺.

### Synthesis A72: Synthesis of 6-[2-cyano-3-[[ethyl(methyl)sulfamoyl]amino]-6-fluoro-phenoxy]-4-oxo-3-[2-(4-piperidyl)ethyl]quinazoline (A-72)

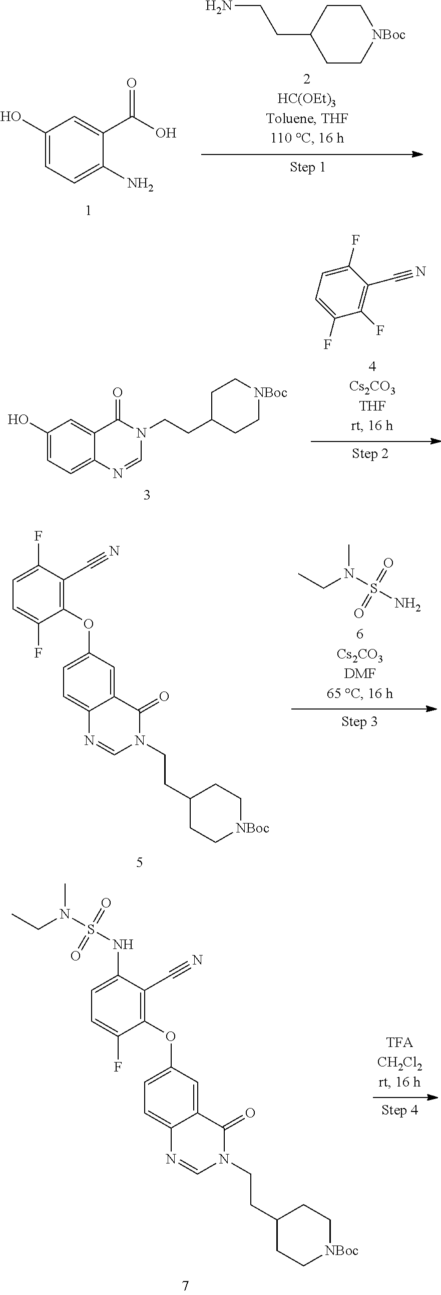

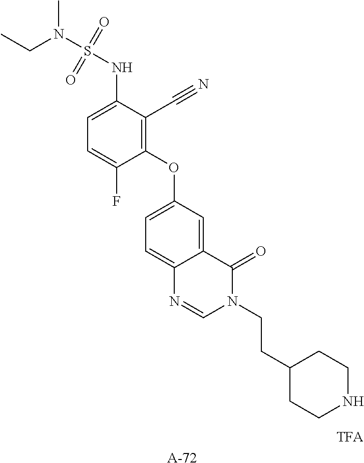 [SMILES: CCN(C)S(=O)(=O)Nc1ccc(F)c(Oc2ccc3ncn(CCC4CCNCC4)c(=O)c3c2)c1C#N ; "TFA" ; "A-72"]

### Step 1

Into 20 mL glass vial with pressure released cap containing a well-stirred solution of tert-butyl 4-(2-aminoethyl)piperidine-1-carboxylate (2, 1.0 g, 4.38 mmol) in THF (4 mL) and toluene (8 mL) was added triethyl orthoformate (11.82 mmol, 1.97 mL), 2-amino-5-hydroxy-benzoic acid (1, 670.67 mg, 4.38 mmol) at room temperature under nitrogen atmosphere. Resulting reaction mixture was stirred at 110° C. for 16 hours. After completion of the reaction, the reaction mixture was quenched with water (20 mL) and extracted with ethyl acetate (3×50 mL). Combined organic layer was washed with saturated NaHCO₃ solution (20 mL) followed by saturated NaCl solution (20 mL), dried over anhydrous Na₂SO₄, filtered and concentrated under reduced pressure to afford a crude residue which was purified by flash (silica-gel 230-400 mesh, 50 g) column chromatography using 0-100% ethyl acetate/hexane as an eluent while desired product was eluted at 60-70% of the mobile phase to afford tert-butyl 4-[2-(6-hydroxy-4-oxo-quinazolin-3-yl)ethyl]piperidine-1-carboxylate (3, 1.0 g, 2.67 mmol, 61% yield) as an off-white solid. UPLC-MS (ES+): m/z 374.2 [M+H]⁺.

### Step 2

Into a 100 mL two-neck round-bottom flask containing a well stirred solution of tert-butyl 4-[2-(6-hydroxy-4-oxo-quinazolin-3-yl)ethyl]piperidine-1-carboxylate (3, 1.0 g, 2.65 mmol) in THF (15 mL) was added Cs₂CO₃ (863.75 mg, 2.65 mmol) and 2,3,6-trifluorobenzonitrile (4, 2.65 mmol, 306.21 μL) at room temperature and resulting reaction mixture was stirred at the same temperature for 16 hours. After completion of the reaction, the reaction mixture was quenched with water (20 mL) and extracted with ethyl acetate (3×60 mL). Combined organic layer was washed with saturated NaCl solution (20 mL), dried over anhydrous Na₂SO₄, filtered and concentrated under reduced pressure to afford a crude residue which was purified by flash (silica-gel 230-400 mesh, 25 g) column chromatography using 0-100% ethyl acetate/hexane as an eluent while desired product was eluted at 60-70% of the mobile phase to afford tert-butyl 4-[2-[6-(2-cyano-3,6-difluoro-phenoxy)-4-oxo-quinazolin-3-yl]ethyl]piperidine-1-carboxylate (5, 1.1 g, 2.09 mmol, 79% yield) as an off-white solid. UPLC-MS (ES+): m/z 455.1 [M−^{t-}Bu+H]⁺.

### Step 3

Into a 100 mL two-neck round-bottom flask containing a well stirred solution of tert-butyl 4-[2-[6-(2-cyano-3,6-difluoro-phenoxy)-4-oxo-quinazolin-3-yl]ethyl]piperidine-1-carboxylate (5, 1.1 g, 2.09 mmol) in N,N-dimethylformamide (10 mL) were added [methyl(sulfamoyl)amino]ethane (6, 433.22 mg, 3.13 mmol) and Cs₂CO₃ (2.04 g, 6.27 mmol) at room temperature. Resulting reaction mixture was stirred at 65° C. for 16 hours. After completion of the reaction, the reaction mixture was quenched with water (50 mL). Precipitated solid was filtered through a Celite pad and pad was washed with water (30 mL) and filtrate was extracted with ethyl acetate (3×100 mL). Combined organic layer was washed with water (50 mL) and saturated NaCl solution (50 mL), dried over anhydrous Na₂CO₃, filtered and concentrated under reduced pressure to afford a crude tert-butyl 4-[2-[6-[2-cyano-3-[[ethyl(methyl)sulfamoyl]amino]-6-fluoro-phenoxy]-4-oxo-quinazolin-3-yl]ethyl]piperidine-1-carboxylate (7, 0.8 g, 1.21 mmol, 58% yield) off-white solid. UPLC-MS (ES+): m/z 627.2 [M−H]⁻.

### Step 4

Into a 100 mL single-neck round bottom flask containing a well stirred solution of tert-butyl 4-[2-[6-[2-cyano-3-[[ethyl(methyl)sulfamoyl]amino]-6-fluoro-phenoxy]-4-oxo-quinazolin-3-yl]ethyl]piperidine-1-carboxylate (7, 800.00 mg, 1.27 mmol) in dichloromethane (10 mL) was added trifluoroacetic acid (6.36 mmol, 486.86 L) at 0° C. Resulting reaction mixture was stirred at room temperature for 16 hours. After completion of the reaction, the reaction mixture was concentrated under reduced pressure to afford a crude 6-[2-cyano-3-[[ethyl(methyl)sulfamoyl]amino]-6-fluoro-phenoxy]-4-oxo-3-[2-(4-piperidyl)ethyl]quinazoline (A-72, 0.6 g, 894.56 mol, 70% yield, TFA salt) as an off-white solid. UPLC-MS (ES+): m/z 529.1 [M+H]⁺.

### Synthesis A73: Synthesis of 7-[2-cyano-3-[[ethyl(methyl)sulfamoyl]amino]-6-fluoro-phenoxy]-2-[2-(4-piperidyl)ethyl]quinoxaline (A-73)

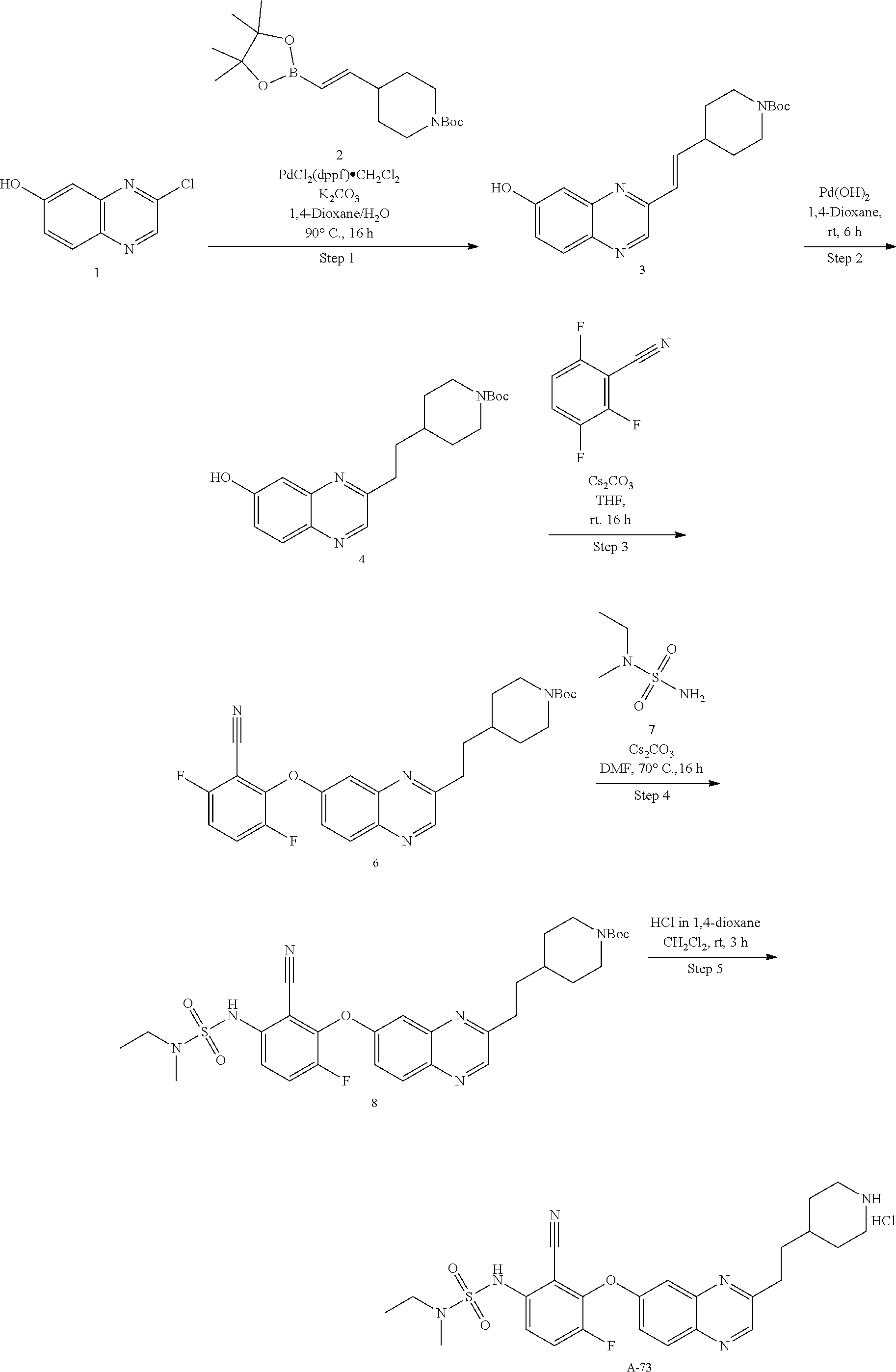

### Step 1

Into a 40 mL pressure vial containing a well-stirred solution of 3-chloroquinoxalin-6-ol (1, 1 g, 5.36 mmol) and tert-butyl 4-[(E)-2-(4,4,5,5-tetramethyl-1,3,2-dioxaborolan-2-yl)vinyl]piperidine-1-carboxylate (2, 2.17 g, 6.43 mmol) in anhydrous 1,4-dioxane (10 mL) and water (3 mL) was added K2CO₃ (1.85 g, 13.41 mmol) at room temperature under nitrogen atmosphere. The reaction mixture was degassed for 10 min with nitrogen and [1,1′-Bis(diphenylphosphino)ferrocene]dichloropalladium(II), complex with dichloromethane (437.91 mg, 536.24 mol) was added. Resulting reaction mixture was stirred at 90° C. for 16 hours. After completion of the reaction, the reaction mixture was cooled to room temperature and poured into water (50 mL) and extracted with ethyl acetate (2×100 mL). Combined organic layers were washed with saturated NaCl (10 mL), dried over anhydrous Na₂SO₄, filtered and concentrated under reduced pressure to get a crude residue which was purified by flash silica-gel (230-400 mesh) column with 0-100% ethyl acetate/hexane to afford tert-butyl 4-[(E)-2-(7-hydroxyquinoxalin-2-yl)vinyl]piperidine-1-carboxylate (3, 1.03 g, 2.76 mmol, 51% yield) as a pale yellow solid. UPLC-MS (ES+): m/z 356.2 [M+H]⁺.

### Step 2

Into a 25 mL single-neck round-bottom flask containing a well-stirred solution of tert-butyl 4-[(E)-2-(7-hydroxyquinoxalin-2-yl)vinyl]piperidine-1-carboxylate (3, 1.03 g, 2.76 mmol) in anhydrous 1,4-dioxane (20 mL) was added Pd(OH)₂ on carbon 10% (310 mg, 2.76 mmol) at room temperature under nitrogen atmosphere. The resulting suspension was stirred at room temperature under hydrogen bladder pressure for 6 hours. After completion of the reaction, the reaction mixture was filtered through a pad of Celite and washed with ethyl acetate (100 mL). Organic layers were concentrated under reduced pressure to get crude tert-butyl 4-[2-(7-hydroxyquinoxalin-2-yl)ethyl]piperidine-1-carboxylate (4, 1.03 g, 2.54 mmol, 92% yield) as brown gummy liquid. UPLC-MS (ES+): m/z 358.2 [M+H]⁺.

### Step 3

Into a 50 mL two-neck round-bottom flask containing a well-stirred solution of tert-butyl 4-[2-(7-hydroxyquinoxalin-2-yl)ethyl]piperidine-1-carboxylate (4, 1.04 g, 2.55 mmol) in anhydrous THF (15 mL) were added Cs₂CO₃ (2.49 g, 7.65 mmol) and 2,3,6-trifluorobenzonitrile (5, 3.06 mmol, 353.48 L) under nitrogen atmosphere and stirred at room temperature for 16 hours. After completion of the reaction, the reaction mixture was cooled to room temperature, diluted with H₂O (10 mL) and washed with ethyl acetate (2×20 mL). Combined organic layers were dried over anhydrous Na₂SO₄, filtered and the filtrate was evaporated under reduced pressure to get a crude residue, tert-butyl 4-[2-[7-(2-cyano-3,6-difluoro-phenoxy)quinoxalin-2-yl]ethyl]piperidine-1-carboxylate (6, 996 mg, 1.91 mmol, 75% yield) as a brown gummy liquid. UPLC-MS (ES+): m/z 439.0 [M+H−^{t-}Bu]⁺.

### Step 4

Into a 50 mL two-neck round-bottom flask containing a well-stirred solution of tert-butyl 4-[2-[7-(2-cyano-3,6-difluoro-phenoxy)quinoxalin-2-yl]ethyl]piperidine-1-carboxylate (6, 990 mg, 1.88 mmol) in anhydrous N,N-dimethylformamide (12 mL) were added Cs₂CO₃ (1.84 g, 5.65 mmol) and methyl(sulfamoyl)amino]ethane (7, 649.21 mg, 4.70 mmol) at room temperature under nitrogen atmosphere. The reaction mixture was stirred at 70° C. for 16 h under nitrogen atmosphere. After completion of the reaction, the reaction mixture was cooled to room temperature and diluted with water (30 mL). Obtained solid was filtered and the filtrate was extracted with ethyl acetate (3×50 mL) and saturated NaCl (20 mL). Combined organic layers were dried over anhydrous Na₂SO₄, filtered and the filtrate was evaporated under reduced pressure to get crude tert-butyl 4-[2-[7-[2-cyano-3-[[ethyl(methyl)sulfamoyl]amino]-6-fluoro-phenoxy]quinoxalin-2-yl]ethyl]piperidine-1-carboxylate (8, 1.1 g, 1.06 mmol, 56% yield) as a brown gummy liquid. UPLC-MS (ES+): m/z 513.1 [M+H-Boc]⁺.

### Step 5

Into a 50 mL single-neck round-bottom flask containing a well-stirred solution of tert-butyl 4-[2-[7-[2-cyano-3-[[ethyl(methyl)sulfamoyl]amino]-6-fluoro-phenoxy]quinoxalin-2-yl]ethyl]piperidine-1-carboxylate (8, 1.12 g, 1.06 mmol) in anhydrous dichloromethane (13 mL) was added hydrogen chloride solution (4.0 M in 1,4-dioxane, 5.30 mmol,1.32 mL) at 0° C. under nitrogen atmosphere. The resulting mixture was stirred at room temperature for 3 hours. After completion of the reaction, solvents were evaporated under reduced pressure to get a crude residue, which was washed with methyl tert-butyl ether to remove the non-polar impurity and get the crude 7-[2-cyano-3-[[ethyl(methyl)sulfamoyl]amino]-6-fluoro-phenoxy]-2-[2-(4-piperidyl)ethyl]quinoxaline (A-73, 755 mg, 1.05 mmol, 99% yield,HCl salt) as a dark brown solid. UPLC-MS (ES+): m/z 513.2 [M+H]⁺.

### Synthesis A74: Synthesis of 7-[2-cyano-3-[[ethyl(methyl)sulfamoyl]amino]-6-fluoro-phenoxyl-2-(4-piperidyloxy)quinoxaline (A-74)

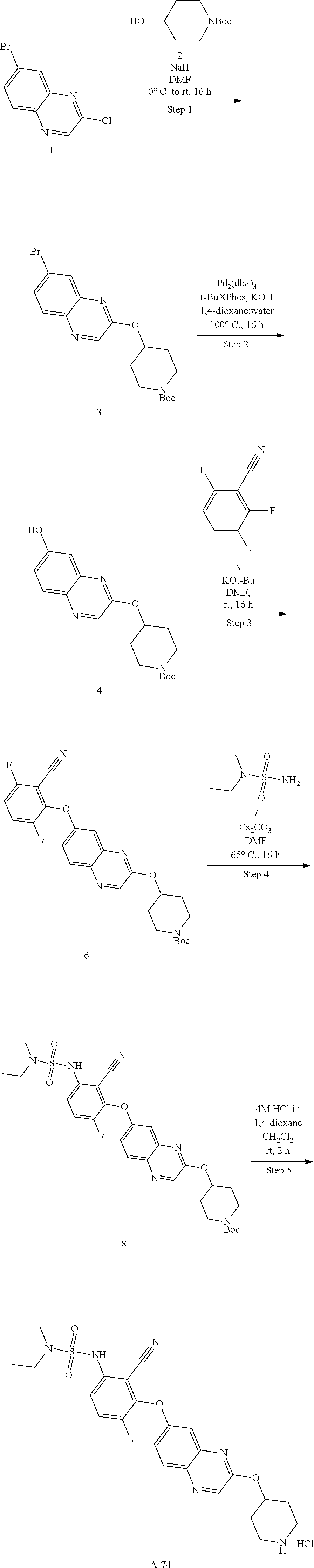

### Step 1

Into a 50 mL two-neck round bottom flask containing a well stirred solution of sodium hydride (in oil dispersion) 60% dispersion in mineral oil (492.84 mg, 20.53 mmol) in N,N-dimethylformamide (40 mL) was added a solution of tert-butyl 4-hydroxypiperidine-1-carboxylate (2, 4.13 g, 20.53 mmol) dissolved in N,N-dimethylformamide (30 mL) at 0° C. under nitrogen atmosphere and resulting mixture was stirred for 20 min at room temperature. To the above mixture, a solution of 7-bromo-2-chloro-quinoxaline (1, 5 g, 20.53 mmol) dissolved in N,N-dimethylformamide (30 mL) was added at 0° C. and resulting reaction mixture was stirred for 16 h at room temperature. After completion of the reaction, the reaction mixture was quenched with ice-cold water (20 mL) and extracted in ethyl acetate (2×300 mL). Combined organic layer was dried over anhydrous Na₂SO₄, filtered and concentrated under reduced pressure to afford a crude residue which was purified by flash (230-400 mesh, 50 g) column chromatography using 0-100% ethyl acetate/hexane as an eluent while desired product was eluted at 10-20% of mobile phase to afford tert-butyl 4-(7-bromoquinoxalin-2-yl)oxypiperidine-1-carboxylate (3, 4.7 g, 11.48 mmol, 56% yield) as an off-white solid. UPLC-MS(ES+): m/z 410.0 [M+H]⁺.

### Step 2

Into a 250 mL sealed tube reactor containing a well stirred solution tert-butyl 4-(7-bromoquinoxalin-2-yl)oxypiperidine-1-carboxylate (3, 4.5 g, 11.02 mmol) in water (30 mL) and 1,4 dioxane (60 mL) was added potassium hydroxide (618.42 mg, 11.02 mmol, 302.56 μL) at room temperature under nitrogen atmosphere and resulting mixture was degassed with nitrogen for 5 min. ^{t-}BuXPhos (234.01 mg, 551.08 mol) was added to the above mixture and degassing was continued for additional 5 min. Subsequently, Tris(dibenzylideneacetone)dipalladium(0) (100.93 mg, 110.22 mol) was added and resulting reaction mixture was stirred at 100° C. for 16 hours. After completion of the reaction, the reaction mixture was diluted with ice-cold water (150 mL) and extracted in ethyl acetate (2×200 mL). Combined organic layer was dried over anhydrous Na₂SO₄, filtered and concentrated under reduced pressure to afford a crude residue which was purified by flash (230-400 mesh, 50 g) column chromatography using 0-100% ethyl acetate/hexane as an eluent while desired product was eluted at 20-60% of mobile phase to afford pure tert-butyl 4-(7-hydroxyquinoxalin-2-yl)oxypiperidine-1-carboxylate (4, 3.75 g, 10.61 mmol, 96% yield) as a yellow solid. UPLC-MS(ES−): m/z 344.2 [M−H]⁻.

### Step 3

Into a 250 mL single-neck round bottom flask containing a well stirred solution of tert-butyl 4-(7-hydroxyquinoxalin-2-yl)oxypiperidine-1-carboxylate (4, 3.7 g, 10.71 mmol) and 2,3,6-trifluorobenzonitrile (5, 15.00 mmol, 1.73 mL) in N,N-dimethylformamide (50 mL), was added potassium tert-butoxide (1.92 g, 17.14 mmol) at 0° C. and resulting reaction mixture was stirred at room temperature for 16 hours. After completion of the reaction, the reaction mixture was diluted with water (100 mL) and extracted in ethyl acetate (2×200 mL). Combined organic layer was dried over anhydrous Na₂SO₄, filtered and concentrated under reduced pressure to afford a crude residue which was purified by flash (230-400 mesh, 50 g) column chromatography using 0-100% ethyl acetate/hexane as an eluent while desired product was eluted at 0-20% of mobile phase to afford tert-butyl 4-[7-(2-cyano-3,6-difluoro-phenoxy)quinoxalin-2-yl]oxypiperidine-1-carboxylate (6, 4.2 g, 7.50 mmol, 70% yield) as a yellow solid. UPLC-MS(ES+): m/z 483.1 [M+H]⁺.

### Step 4

Into a two-neck round-bottom flask containing a well-stirred solution of tert-butyl 4-[7-(2-cyano-3,6-difluoro-phenoxy)quinoxalin-2-yl]oxypiperidine-1-carboxylate (6, 4 g, 8.29 mmol) in N,N-dimethylformamide (80 mL) were added [methyl(sulfamoyl)amino]ethane (7, 2.29 g, 16.58 mmol) and cesium carbonate (8.10 g, 24.87 mmol) at room temperature under nitrogen atmosphere. Resulting reaction mixture was stirred at 65° C. for 16 hours. After completion of the reaction, the reaction mixture was quenched with water (100 mL) and precipitated solid was filtered through a celite pad, and the celite pad was washed with water (50 mL). Filtrate was extracted with ethyl acetate (3×200 mL) and combined organic layer was washed with water (50 mL), saturated NaCl solution (50 mL), dried over anhydrous Na₂SO₄, filtered and concentrated under reduced pressure to afford a crude tert-butyl 4-[7-[2-cyano-3-[[ethyl(methyl)sulfamoyl]amino]-6-fluoro-phenoxy]quinoxalin-2-yl]oxypiperidine-1-carboxylate (8, 4.1 g, 4.81 mmol, 58% yield) as a brown thick liquid. UPLC-MS (ES−): m/z 599.3 [M−H]⁻.

### Step 5

Into a 100 mL single-neck round-bottom flask containing a well-stirred solution of tert-butyl-4-[7-[2-cyano-3-[[ethyl(methyl)sulfamoyl]amino]-6-fluoro-phenoxy]quinoxalin-2-yl]oxypiperidine-1-carboxylate (8, 2 g, 2.35 mmol) in dichloromethane (30 mL) was added HCl in 1,4-dioxane (4 M, 8.21 mL) at 0° C. temperature. Resulting reaction mixture was stirred at room temperature for 2 hours. After completion of the reaction, the reaction mixture was concentrated under reduced pressure to afford a crude residue which was triturated with methyl tert-butyl ether (80 mL) to afford crude 7-[2-cyano-3-[[ethyl(methyl)sulfamoyl]amino]-6-fluoro-phenoxy]-2-(4-piperidyloxy)quinoxaline (A-74, 1.4 g, 1.90 mmol, 81% yield, HCl salt) as a brown thick liquid. UPLC-MS (ES+): m/z 501.3[M+H]⁺.

### Synthesis A75: Synthesis of 7-[2-cyano-3-[[ethyl(methyl)sulfamoyl]amino]-6-fluoro-phenoxy]-2-(4-piperidylmethyl)quinoxaline (A-75)

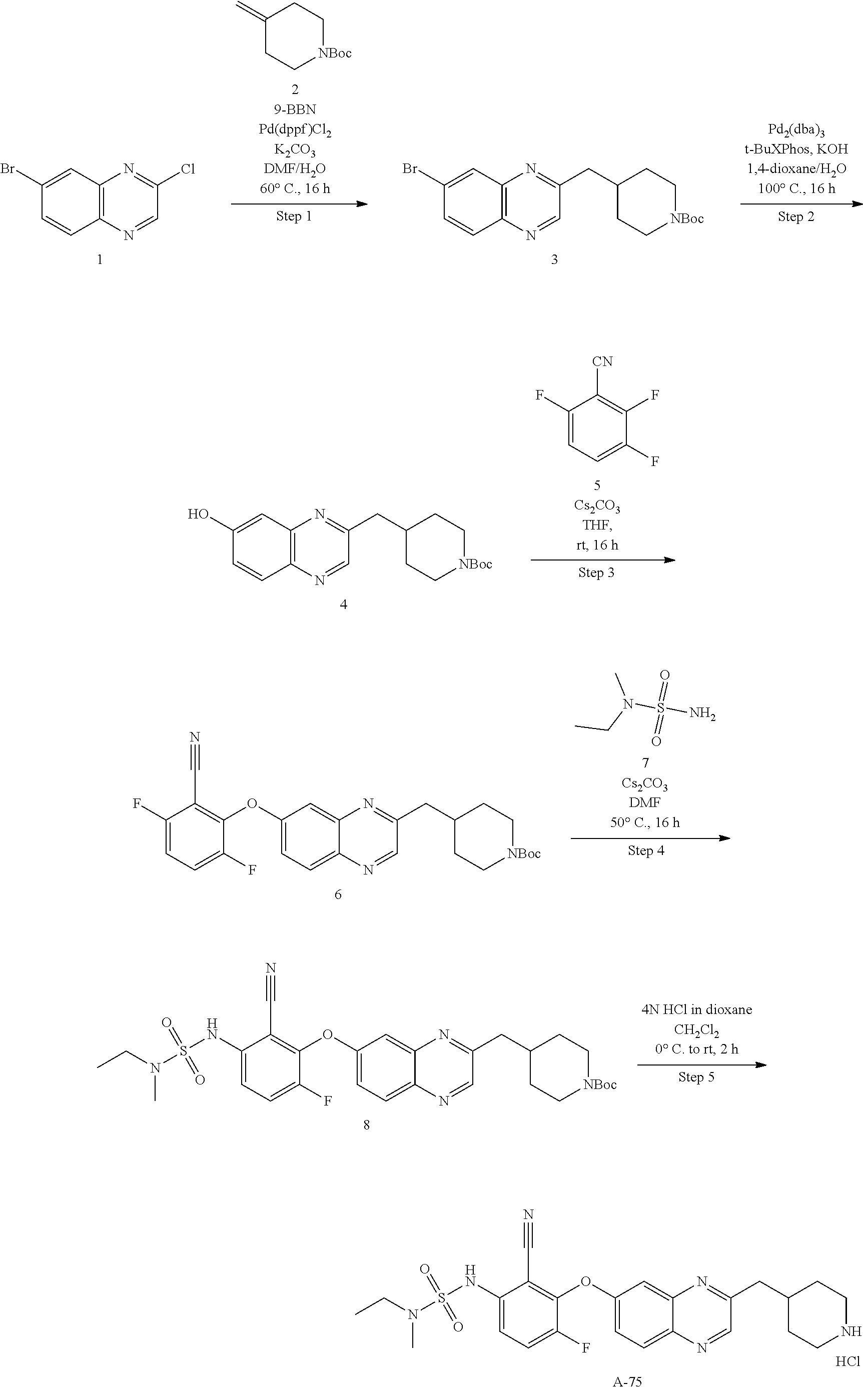

### Step 1

A: Preparation of Boronate Solution: Into a 250 mL single-neck round-bottom flask containing a well-stirred solution of tert-butyl 4-methylenepiperidine-1-carboxylate (2, 8.10 g, 41.07 mmol, 8.10 mL) was added 9-BBN, 0.5 M in THF (0.5 M, 82.14 mL) at room temperature under nitrogen atmosphere. Reaction mixture was stirred at 60° C. for 1 hour. Finally, reaction mixture was allowed to room temperature

B: Suzuki Coupling Reaction: Into a 250 mL two-neck round-bottom flask containing a well-stirred solution of 7-bromo-2-chloro-quinoxaline (1, 5.0 g, 20.53 mmol) in N,N-dimethylformamide (25 mL) and water (5 mL) were added K2CO₃ (3.12 g, 22.59 mmol) and Pd(dppf)Cl₂ (751.27 mg, 1.03 mmol) and stirred for 10 min. After 10 min of stirring, freshly prepared boronate solution was added dropwise to the above reaction mixture under nitrogen atmosphere. The reaction mixture was stirred at 60° C. for 16 hours. After completion of the reaction, the reaction mixture was brought down to room temperature and poured into 10% NaHCO₃ solution (25 mL). Aqueous layer was extracted with dichloromethane (2×80 mL) and combined organic layers were washed with saturated NaCl (50 mL), dried over anhydrous Na₂SO₄, filtered and filtrate was concentrated under reduced pressure to get a crude residue which was purified by flash silica-gel (230-400 mesh) column with 0-100% ethyl acetate/hexane to afford tert-butyl 4-[(7-bromoquinoxalin-2-yl)methyl]piperidine-1-carboxylate (3, 2.8 g, 5.79 mmol, 28% yield) as a brown colored gum. UPLC-MS (ES+): m/z 406.0 [M+H]⁺.

### Step 2

Into a 50 mL sealed tube containing a well stirred solution of H₂O (4 mL), was added KOH (580.00 mg, 10.34 mmol, 283.76 L) at 10° C. and stirred for 10 min. To the above mixture, 1,4-dioxane (16 mL), tert-butyl 4-[(7-bromoquinoxalin-2-yl)methyl]piperidine-1-carboxylate (3, 2.0 g, 4.13 mmol) and ^{t-}BuXPhos (175.58 mg, 413.47 mol) were added. The mixture was purged with nitrogen for 10 min and Pd₂(dba)₃ (378.62 mg, 413.47 mol) was added. Reaction mixture was stirred at 100° C. for 16 hours. After completion of the reaction, the reaction mixture was allowed to cool to room temperature and diluted with 1M KOH solution (20 mL) and ethyl acetate (60 mL). The layers were separated, and the pH of the aqueous layer was adjusted to ~3 using 1.5N HCl (15 mL) solution. Aqueous layer was extracted with dichloromethane (2×40 mL) and the combined organic layers were washed with saturated NaCl (50 mL), dried over anhydrous Na₂SO₄, filtered and filtrate was concentrated under reduced pressure to afford tert-butyl 4-[(7-hydroxyquinoxalin-2-yl)methyl]piperidine-1-carboxylate (4, 700 mg, 1.98 mmol, 48% yield) as a pale yellow solid. UPLC-MS (ES+): m/z 344.2 [M+H]⁺.

### Step 3

Into a 50 mL single-neck round-bottom flask containing a well-stirred solution of tert-butyl 4-[(7-hydroxyquinoxalin-2-yl)methyl]piperidine-1-carboxylate (4, 600 mg, 1.75 mmol) and 2,3,6-trifluorobenzonitrile (5, 2.10 mmol, 242.17 μL) in anhydrous THF (12.00 mL) was added Cs₂CO₃ (1.71 g, 5.24 mmol) at room temperature and the resulting suspension was stirred at room temperature for 16 hours. After completion of the reaction, the reaction mixture was diluted with water (30 mL) and extracted with dichloromethane (2×80 mL). Combined organic layers were dried (anhydrous Na₂SO₄), filtered and the filtrate was concentrated under reduced pressure to get a crude residue, which was purified by flash silica-gel (230-400 mesh) column with 0-100% ethyl acetate/hexane to afford tert-butyl 4-[[7-(2-cyano-3,6-difluoro-phenoxy)quinoxalin-2-yl]methyl]piperidine-1-carboxylate (6, 800 mg, 1.63 mmol, 93% yield) as a pale yellow colored solid. UPLC-MS (ES+): m/z 425.0 [M+H]⁺.

### Step 4

Into a 100 mL single-neck round-bottom flask containing a well-stirred solution of tert-butyl 4-[[7-(2-cyano-3,6-difluoro-phenoxy)quinoxalin-2-yl]methyl]piperidine-1-carboxylate (6, 1.4 g, 2.91 mmol) and [methyl(sulfamoyl)amino]ethane (7, 805.25 mg, 5.83 mmol) in anhydrous N,N-dimethylformamide (14 mL) was added Cs₂CO₃ (2.37 g, 7.28 mmol) at room temperature and the resulting suspension was stirred at 50° C. for 16 hours. After completion of the reaction, the reaction mixture was diluted with water (30 mL) and extracted with dichloromethane (2×80 mL). Combined organic layers were dried (anhydrous Na₂SO₄), filtered and the filtrate was concentrated under reduced pressure to get a crude residue, which was purified by flash silica-gel (230-400 mesh) column with 0-100% ethyl acetate/hexane to afford tert-butyl 4-[[7-[2-cyano-3-[[ethyl(methyl)sulfamoyl]amino]-6-fluoro-phenoxy]quinoxalin-2-yl]methyl]piperidine-1-carboxylate (8, 860 mg, 1.32 mmol, 45% yield) as a pale yellow colored gum. UPLC-MS (ES+): m/z 597.2 [M−H]⁻.

### Step 5

Into a 50 mL single-neck round-bottom flask containing a well-stirred solution of tert-butyl 4-[[7-[2-cyano-3-[[ethyl(methyl)sulfamoyl]amino]-6-fluoro-phenoxy]quinoxalin-2-yl]methyl]piperidine-1-carboxylate (8, 800 mg, 1.34 mmol) in anhydrous DCM (8 mL) was added 4 M HCl in 1,4-dioxane (4 M, 4.00 mL) slowly at 0° C. under nitrogen atmosphere. The resulting solution was stirred at room temperature for 2 hours. After completion of the reaction, the reaction mixture was concentrated under reduced pressure and the obtained residue was washed with methyl tert-butyl ether to remove the non-polar impurity to afford 7-[2-cyano-3-[[ethyl(methyl)sulfamoyl]amino]-6-fluoro-phenoxy]-2-(4-piperidylmethyl)quinoxaline (A-75, 700 mg, 1.28 mmol, 96% yield, HCl salt) as an off-white solid. UPLC-MS (ES+): m/z 499.0 [M+H]⁺.

### Synthesis A76: Synthesis of 2-(2-azaspiro[3.3]heptan-6-yloxy)-7-[2-cyano-3-[[ethyl(methyl)sulfamoyl]amino]-6-fluoro-phenoxy]quinoxaline (A-76)

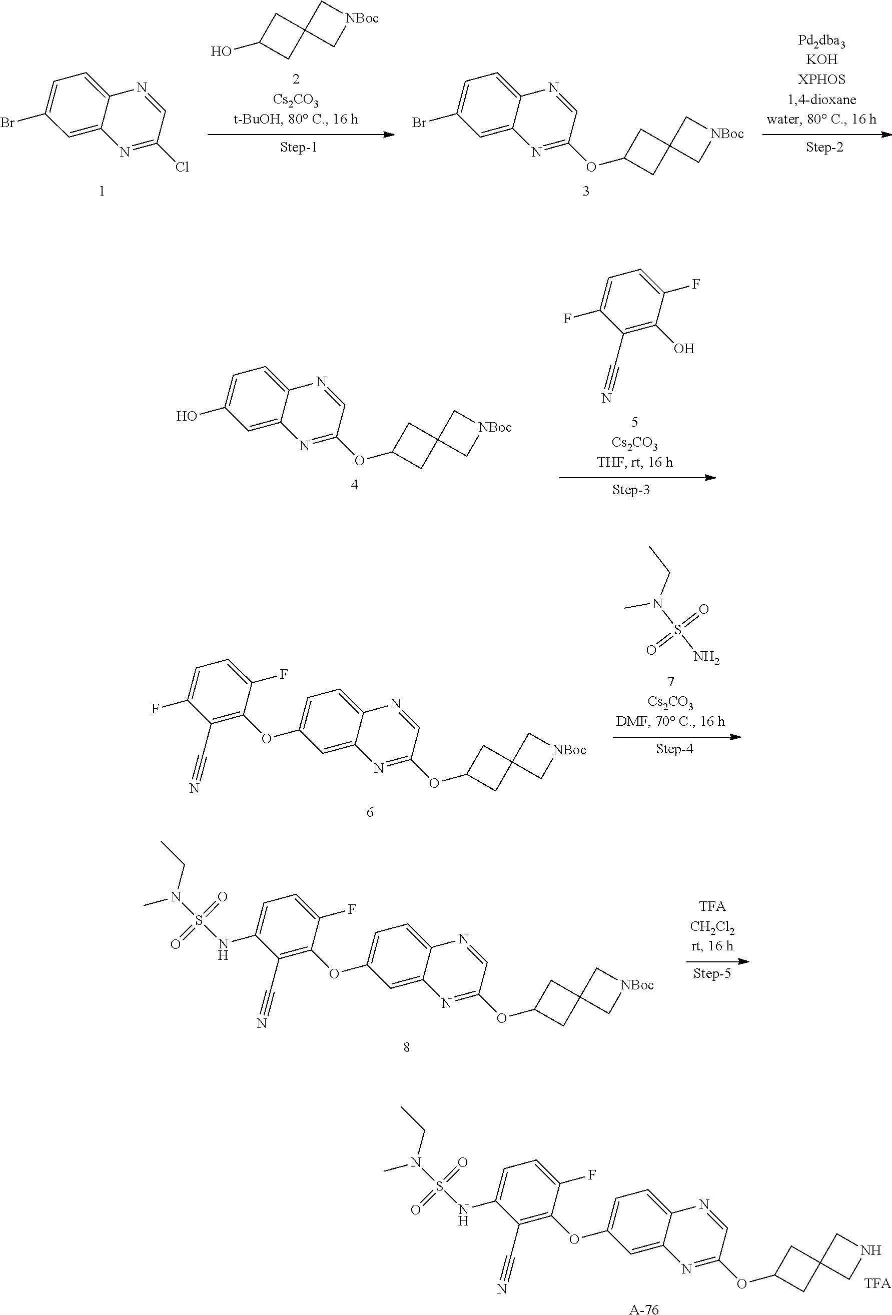

### Step 1

Into a 250 mL single neck round bottom flask containing a well stirred solution of tert-butyl 6-hydroxy-2-azaspiro[3.3]heptane-2-carboxylate (2, 3 g, 14.07 mmol) in t-BuOH (100 mL) were added cesium carbonate (11.46 g, 35.17 mmol) and 7-bromo-2-chloro-quinoxaline (1, 3.77 g, 15.47 mmol) and the reaction mixture was heated at 80° C. for 16 h. After completion, the reaction mixture was dilute with ethyl acetate (150 mL), washed with water (150 mL), dried over Na₂SO₄ and concentrated under reduced pressure to afford crude residue which was purified by flash silica-gel (230-400 mesh) column with 0-100% EtOH/hexane to afford tert-butyl 6-(7-bromoquinoxalin-2-yl)oxy-2-azaspiro[3.3]heptane-2-carboxylate (3, 5.4 g, 12.64 mmol, 90% yield) as a gummy liquid. UPLC-MS (ES+): m/z 422.0 [M+H]⁺.

### Step 2

Into a 40 mL pressure vial containing a well-stirred solution of KOH (328.48 mg, 5.85 mmol, 160.70 L) in Water (5 mL) was cooled to 10° C. and added 1,4-dioxane (15 mL) followed by tert-butyl 6-(7-bromoquinoxalin-2-yl)oxy-2-azaspiro[3.3]heptane-2-carboxylate (3, 1 g, 2.34 mmol) and ^{t-}BuXPhos (99.44 mg, 234.17 mol) at same temperature, reaction mixture was purged with nitrogen for 15 minutes. Pd₂dba₃ (214.43 mg, 234.17 mol) was added and stirred at 80° C. for 16 hours. After completion of the reaction, the reaction mixture was diluted with water (50 mL) and extracted with ethyl acetate (3×50 mL), dried over Na₂SO₄ and concentrated under reduced pressure to get a crude residue which was purified by flash silica-gel (230-400 mesh) column with 0-100% ethyl acetate/Hexane, while compound eluting at 35-40% to afford tert-butyl 6-(7-hydroxyquinoxalin-2-yl)oxy-2-azaspiro[3.3]heptane-2-carboxylate (4, 740 mg, 2.04 mmol, 87% yield) as an off-white solid. UPLC-MS (ES+): m/z 358.3 [M+H]⁺.

### Step 3

Into a 40 mL pressure vial containing a well stirred solution of tert-butyl 6-(7-hydroxyquinoxalin-2-yl)oxy-2-azaspiro[3.3]heptane-2-carboxylate (4, 751.80 mg, 2.07 mmol) in anhydrous THF (20 mL) were added Cs₂CO₃ (2.02 g, 6.21 mmol) and 2,3,6-trifluorobenzonitrile (5, 2.48 mmol, 286.99 L,) and stirred at room temperature for 16 hours. After completion of the reaction, the reaction mixture was diluted with water (50 mL), washed with ethyl acetate (3×50 mL), dried over Na₂SO₄ and concentrated under reduced pressure to get crude, which was purified by flash silica-gel (100-200 mesh) column chromatography with 0-100% ethyl acetate/hexane to afford tert-butyl 6-[7-(2-cyano-3,6-difluoro-phenoxy)quinoxalin-2-yl]oxy-2-azaspiro[3.3]heptane-2-carboxylate (6, 1 g, 2.01 mmol, 97% yield) as a gummy liquid. UPLC-MS (ES+): m/z 495.1 [M+H]⁺.

### Step 4

Into a 40 mL pressure vial containing a well stirred solution of tert-butyl 6-[7-(2-cyano-3,6-difluoro-phenoxy)quinoxalin-2-yl]oxy-2-azaspiro[3.3]heptane-2-carboxylate (6, 917.40 mg, 1.84 mmol) in N,N-dimethylformamide (18 mL) were added Cs₂CO₃ (1.80 g, 5.53 mmol) and [methyl(sulfamoyl)amino]ethane (7, 670.62 mg, 4.61 mmol) at room temperature. The reaction mixture was stirred at 70° C. for 16 hours. After completion of the reaction, the reaction mixture was diluted with water (30 mL) and extracted with ethyl acetate (3×50 mL), the combined organic layers were dried over Na₂SO₄ and concentrated under reduced pressure to afford crude tert-butyl 6-[7-[2-cyano-3-[[ethyl(methyl)sulfamoyl]amino]-6-fluoro-phenoxy]quinoxalin-2-yl]oxy-2-azaspiro[3.3]heptane-2-carboxylate (8, 900 mg, 1.26 mmol, 68% yield) as pale yellow liquid. UPLC-MS (ES+): m/z 613.2 [M+H]⁺.

### Step 5

Into a 100 mL single-neck round-bottom flask containing a well-stirred solution of tert-butyl 6-[7-[2-cyano-3-[[ethyl(methyl)sulfamoyl]amino]-6-fluoro-phenoxy]quinoxalin-2-yl]oxy-2-azaspiro[3.3]heptane-2-carboxylate (8, 900 mg, 1.26 mmol) in dichloromethane (25 mL) was added trifluoroacetic acid (25.21 mmol, 1.93 mL) slowly at 0° C. under nitrogen atmosphere. The resulting solution was stirred at room temperature for 16 hours. After completion of the reaction, the reaction mixture was concentrated directly under reduced pressure and washed with methyl tert-butyl ether to afford 2-(2-azaspiro[3.3]heptan-6-yloxy)-7-[2-cyano-3-[[ethyl(methyl)sulfamoyl]amino]-6-fluoro-phenoxy]quinoxaline (A-76, 650 mg, 981.36 mol, 78% yield, TFA salt) as pale yellow solid. UPLC-MS (ES+): m/z 513.2 [M+H]⁺.

### Synthesis A77: Synthesis of 6-[2-cyano-3-[[ethyl(methyl)sulfamoyl]amino]-6-fluoro-phenoxy]-4-oxo-3-(4-piperidyl)quinazoline (A-77)

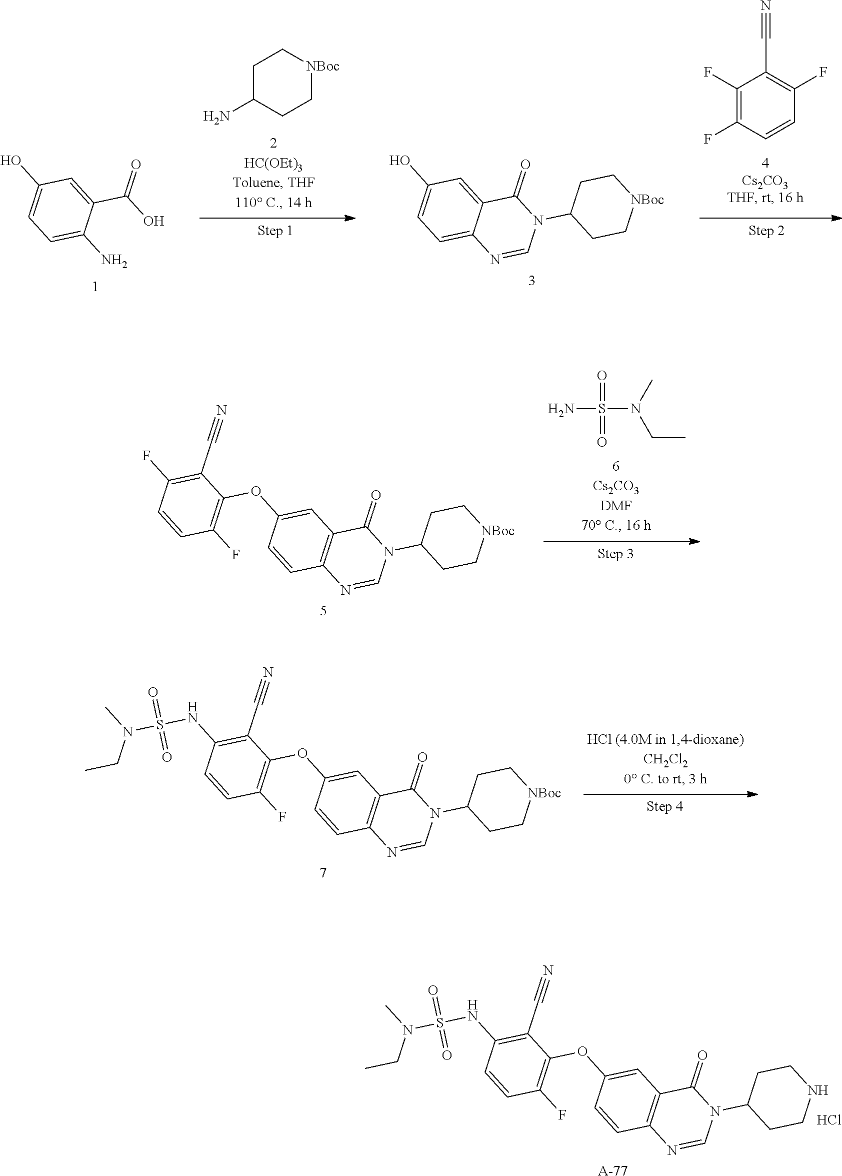

### Step 1

Into a 250 mL three-neck round-bottom flask containing a well stirred solution of 2-amino-5-hydroxy-benzoic acid (1, 5 g, 32.65 mmol), tert-butyl 4-aminopiperidine-1-carboxylate (2, 7.85 g, 39.18 mmol) in Toluene (90 mL) and THF (30 mL) was added triethyl orthoformate (130.60 mmol, 21.72 mL) and resulting mixture stirred at 110° C. for 14 hours. After completion of the reaction, the reaction mixture was diluted with water (150 mL) and extracted with ethyl acetate (3×200 mL). Combined organic layers were washed with NaHCO₃ solution (100 mL), saturated NaCl (100 mL), dried over anhydrous Na₂SO₄ and concentrated under reduced pressure to get crude residue, which was triturated with 5% ethyl acetate/hexane to afford tert-butyl 4-(6-hydroxy-4-oxo-quinazolin-3-yl)piperidine-1-carboxylate (3, 4.5 g, 12.01 mmol, 37% yield) as a brown solid. UPLC-MS (ES+): m/z 346.1 [M+H]⁺.

### Step 2

Into a 250 mL three-neck round-bottom flask containing a well stirred solution of tert-butyl 4-(6-hydroxy-4-oxo-quinazolin-3-yl)piperidine-1-carboxylate (3, 3.5 g, 9.34 mmol) in THF (60 mL) were added Cs₂CO₃ (9.13 g, 28.02 mmol) and 2,3,6-trifluorobenzonitrile (4, 11.21 mmol, 1.29 mL) at room temperature under nitrogen atmosphere. The reaction mixture was stirred at room temperature for 16 hours. After completion of the reaction, the reaction mixture was diluted with water (60 mL), extracted with ethyl acetate (2×100 mL). The organic layers were washed with saturated NaCl solution (150 mL), dried over anhydrous Na₂SO₄ and concentrated under reduced pressure to get crude residue, which was purified by silica gel column chromatography (100-200 mesh) using a gradient of 0-100% ethyl acetate/hexane to afford tert-butyl 4-[6-(2-cyano-3,6-difluoro-phenoxy)-4-oxo-quinazolin-3-yl]piperidine-1-carboxylate (5, 4.2 g, 8.47 mmol, 91% yield) as a brown solid. UPLC-MS (ES+): m/z 483.3 [M+H]⁺.

### Step 3

Into a 40 mL pressure vial containing a well stirred solution of tert-butyl 4-[6-(2-cyano-3,6-difluoro-phenoxy)-4-oxo-quinazolin-3-yl]piperidine-1-carboxylate (5, 2 g, 4.15 mmol) and [methyl(sulfamoyl)amino]ethane (6, 1.15 g, 8.29 mmol) in N,N-dimethylformamide (40 mL) was added Cs₂CO₃ (4.05 g, 12.44 mmol) at room temperature under nitrogen atmosphere. Reaction mixture was stirred at 70° C. for 16 hours. After completion of the reaction, the reaction mixture was quenched with ice cold water (60 mL) and filtered through a celite pad, filtrate was extracted with ethyl acetate (2×80 mL). Combined organic layers were dried over anhydrous Na₂SO₄, concentrated under reduced pressure to afford crude tert-butyl 4-[6-[2-cyano-3-[[ethyl(methyl)sulfamoyl]amino]-6-fluoro-phenoxy]-4-oxo-quinazolin-3-yl]piperidine-1-carboxylate (7, 1.6 g, 1.93 mmol, 47% yield) as a brown sticky liquid. UPLC-MS (ES+): m/z 601.2 [M+H]⁺.

### Step 4

Into a 25 mL single-neck round-bottom flask containing a well stirred solution of tert-butyl 4-[6-[2-cyano-3-[[ethyl(methyl)sulfamoyl]amino]-6-fluoro-phenoxy]-4-oxo-quinazolin-3-yl]piperidine-1-carboxylate (7, 1.5 g, 2.50 mmol) in dichloromethane (15 mL) was added HCl (4.0 M in 1,4-dioxane) (49.95 mmol, 1.73 mL) at 0° C. under nitrogen atmosphere. The reaction mixture was stirred at room temperature for 3 hours. After completion of the reaction, the reaction mixture was concentrated under reduced pressure and co-distilled with dichloromethane (50 mL) to afford 6-[2-cyano-3-[[ethyl(methyl)sulfamoyl]amino]-6-fluoro-phenoxy]-4-oxo-3-(4-piperidyl)quinazoline (A-77, 1.1 g, 1.80 mmol, 72% yield, HCl salt) as a white solid. UPLC-MS (ES+): m/z 501.20 [M+H]⁺.

### Synthesis A78: Synthesis of 3-(2-azaspiro[3.3]heptan-6-yl)-6-[3-[[ethyl(methyl)sulfamoyl]amino]-2,6-difluoro-benzoyl]-4-oxo-quinazoline (A-78)

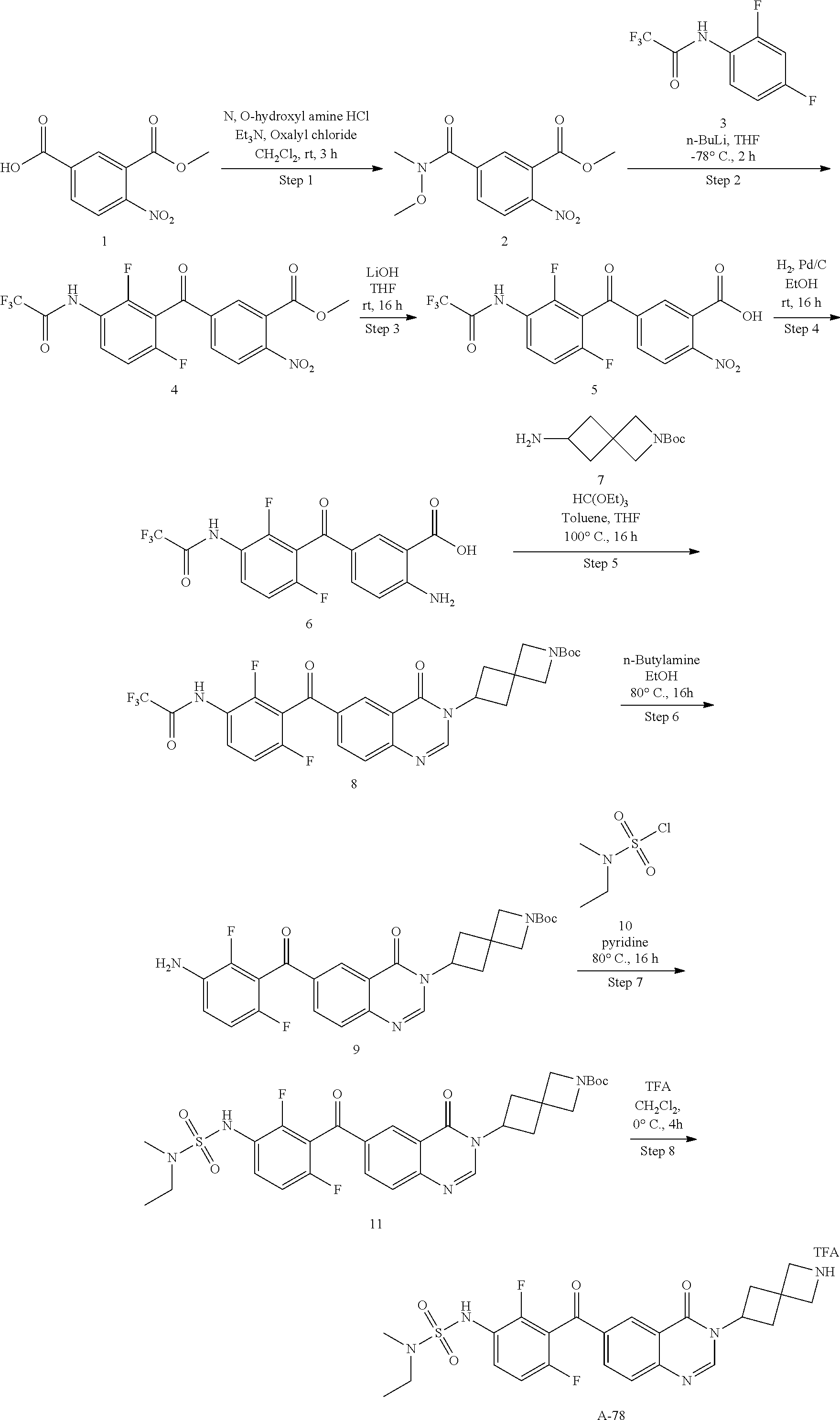

### Step 1

Into a 1 L three-neck round-bottom flask containing a well-stirred solution of 3-methoxycarbonyl-4-nitro-benzoic acid (1, 15.0 g, 66.62 mmol) in dichloromethane (250 mL) were added triethylamine (199.86 mmol, 27.86 mL) and N,N-dimethylformamide (66.62 mol, 5.16 L) at 0° C. under nitrogen atmosphere and resulting reaction mixture was stirred at room temperature for 3 hours. To the above mixture, oxalyl chloride (79.95 mmol, 6.97 mL) and N,O-dimethyl hydroxylamine hydrochloride (6.82 g, 69.95 mmol) were added and stirred at room temperature for 6 hours. After completion of the reaction, the reaction mixture was poured into cold water (350 mL) and extracted with dichloromethane (2×250 mL). Combined organic layers were washed with water (10 mL), saturated NaCl (10 mL), dried over Na₂SO₄ and filtered, filtrates were concentrated under reduced pressure to get crude residue which was purified by flash (100-200 silica mesh) column chromatography using 0-100% ethyl acetate/hexane as mobile phase to afford methyl 5-[methoxy(methyl)carbamoyl]-2-nitro-benzoate (2, 17.0 g, 60.21 mmol, 90% yield) as a brown liquid. ¹H NMR (400 MHz, DMSO-d₆): δ 8.14 (d, J=8.40 Hz, 1H), 8.04 (d, J=1.60 Hz, 1H), 8.00 (dd, J=1.60, 8.20 Hz, 1H), 3.88 (s, 3H), 3.56 (s, 3H), 3.33 (s, 3H).

### Step 2

Into a 100 mL three-neck round-bottom flask containing a well stirred solution of N-(2,4-difluorophenyl)-2,2,2-trifluoro-acetamide (3, 12 g, 53.31 mmol) in THF (120 mL) was added n-BuLi (2.5 M in hexane) (2.5 M, 46.91 mL) at −78° C. and resulting reaction mixture was stirred at the same temperature for 1 hour. A solution of methyl 5-[methoxy(methyl)carbamoyl]-2-nitro-benzoate (2, 15.01 g, 55.97 mmol) dissolved in THF (10 mL) was added to the reaction mixture at −78° C. and stirred at −78° C. for 2 hours. After 2 h, temperature of the reaction mixture was raised to room temperature and stirred for 2 hours. After completion of the reaction, the reaction mixture was quenched with saturated aqueous NH₄C₁ solution (15 mL) and extracted with ethyl acetate (2×50 mL). Combined organic layers were washed with water (10 mL), saturated NaCl (10 mL), dried over Na₂SO₄ and filtered, filtrate was concentrated under reduced pressure to afford methyl 5-[2,6-difluoro-3-[(2,2,2-trifluoroacetyl)amino]benzoyl]-2-nitro-benzoate (4, 16 g, 27.91 mmol, 52% yield) as a gummy liquid. UPLC-MS (ES+): m/z 431.1 [M+H]⁺.

### Step 3

Into a 250 mL single-neck round-bottom flask containing a well stirred solution of methyl 5-[2,6-difluoro-3-[(2,2,2-trifluoroacetyl)amino]benzoyl]-2-nitro-benzoate (4, 8 g, 18.51 mmol) in THF (80 mL) and water (20 mL) was added LiOH·H₂O (1.16 g, 27.76 mmol) portion wise at 0° C. Resulting reaction mixture was stirred at room temperature for 16 hours. After completion of the reaction, the reaction mixture was acidified with 1.5 N HCl and extracted with ethyl acetate (2×100 mL). Combined organic layers were dried over Na₂SO₄ and filtered, filtrates were concentrated under reduced pressure to afford 5-[2,6-difluoro-3-[(2,2,2-trifluoroacetyl)amino]benzoyl]-2-nitro-benzoic acid (5, 6.4 g, 6.66 mmol, 36% yield) as a brown solid. UPLC-MS (ES−): m/z 417.0 [M−H]⁻.

### Step 4

Into a 25 mL single-neck round-bottom flask containing a well-stirred solution of 5-[2,6-difluoro-3-[(2,2,2-trifluoroacetyl)amino]benzoyl]-2-nitro-benzoic acid (5, 6.4 g, 15.30 mmol) in EtOH (10 mL) was added palladium, 10% on carbon, Type 487, dry (3.26 g, 30.61 mmol) at room temperature. The reaction mixture was stirred at room temperature under hydrogen bladder pressure for 16 hours. After completion of the reaction, the reaction mixture was filtered through celite pad and washed with methanol (20 mL), filtrate was concentrated under reduced pressure to afford 2-amino-5-[2,6-difluoro-3-[(2,2,2-trifluoroacetyl)amino]benzoyl]benzoic acid (6, 4.5 g, 3.95 mmol, 26% yield) as a pale brown semi solid. UPLC-MS (ES+): m/z 389.1 [M+H]⁺.

### Step 5

Into a 100 mL single-neck round-bottom flask containing a well-stirred solution of 2-amino-5-[2,6-difluoro-3-[(2,2,2-trifluoroacetyl)amino]benzoyl]benzoic acid (6, 1.25 g, 3.22 mmol) in toluene (16 mL) and THF (4 mL) were added tert-butyl 6-amino-2-azaspiro[3.3]heptane-2-carboxylate (7, 751.83 mg, 3.54 mmol) and triethyl orthoformate (7.08 mmol, 1.18 mL). Resulting reaction mixture was stirred at 100° C. for 16 hours. After completion of the reaction, the reaction mixture was diluted with water (100 mL) and extracted with ethyl acetate (2×100 mL). Combined organic layers were dried over Na₂SO₄ and filtered, filtrate was concentrated under reduced pressure to afford tert-butyl 6-[6-[2,6-difluoro-3-[(2,2,2-trifluoroacetyl)amino]benzoyl]-4-oxo-quinazolin-3-yl]-2-azaspiro[3.3]heptane-2-carboxylate (8, 1.25 g, 564.97 mol, 18% yield) as an off-white solid. UPLC-MS (ES+): m/z 591.1 [M+H]⁺.

### Step 6

Into a 40 mL glass pressure vial containing a well-stirred solution tert-butyl 6-[6-[2,6-difluoro-3-[(2,2,2-trifluoroacetyl)amino]benzoyl]-4-oxo-quinazolin-3-yl]-2-azaspiro[3.3]heptane-2-carboxylate (8, 1.0 g, 1.69 mmol) in EtOH (10 mL) was added n-butylamine (10.13 mmol, 1.00 mL) at room temperature and resulting reaction mixture stirred at 80° C. for 16 hours. After completion of the reaction, the reaction mixture was concentrated under reduced pressure to get crude residue which was purified by flash (100-200 silica mesh) column chromatography using 0-100% ethyl acetate/hexane as mobile phase to afford tert-butyl 6-[6-(3-amino-2,6-difluoro-benzoyl)-4-oxo-quinazolin-3-yl]-2-azaspiro[3.3]heptane-2-carboxylate (9, 700 mg, 1.19 mmol, 70% yield) as a light-yellow solid. UPLC-MS (ES+): m/z 497.2 [M+H]⁺.

### Step 7

Into a 20 mL glass vail containing well-stirred solution of tert-butyl 6-[6-(3-amino-2,6-difluoro-benzoyl)-4-oxo-quinazolin-3-yl]-2-azaspiro[3.3]heptane-2-carboxylate (9, 700 mg, 1.41 mmol) in pyridine (8 mL) was added N-ethyl-N-methyl-sulfamoyl chloride (10, 4.23 mmol, 521.52 L) under nitrogen atmosphere and resulting reaction mixture was stirred at 80° C. for 16 hours. After completion of the reaction, the reaction mixture was concentrated under reduced pressure to get crude residue which was purified by flash (100-200 silica mesh) column using 0-100% ethyl acetate/hexane as mobile phase, while desired product eluted at 80-85% to afford tert-butyl 6-[6-[3-[[ethyl(methyl)sulfamoyl]amino]-2,6-difluoro-benzoyl]-4-oxo-quinazolin-3-yl]-2-azaspiro[3.3]heptane-2-carboxylate (11, 450 mg, 516.54 mol, 37% yield) as an pale-yellow solid. UPLC-MS (ES+): m/z 616.1 [M+H]⁺.

### Step 8

Into a 50 mL single-neck round-bottom flask containing a well-stirred solution of tert-butyl 6-[6-[3-[[ethyl(methyl)sulfamoyl]amino]-2,6-difluoro-benzoyl]-4-oxo-quinazolin-3-yl]-2-azaspiro[3.3]heptane-2-carboxylate (11, 450 mg, 728.55 mol) in dichloromethane (5 mL) was added trifluoroacetic acid (7.29 mmol, 557.53 L) at 0° C. under nitrogen atmosphere. The reaction mixture was stirred at room temperature for 4 hours. After completion of the reaction, solvents were concentrated under reduced pressure and triturated with methyl tert-butyl ether (2×20 mL) to afford 3-(2-azaspiro[3.3]heptan-6-yl)-6-[3-[[ethyl(methyl)sulfamoyl]amino]-2,6-difluoro-benzoyl]-4-oxo-quinazoline (A-78, 440 mg, 630.56 mol, 87% yield, TFA salt) as a yellow gum. UPLC-MS (ES+): m/z 518.0 [M+H]⁺.

### Synthesis A79: Synthesis of (3R,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-79)

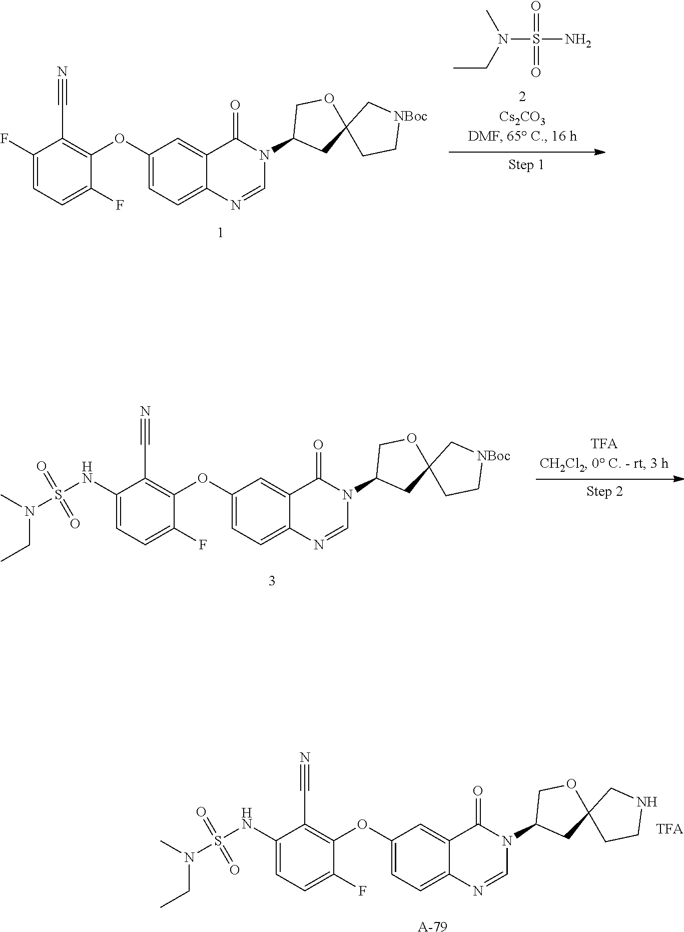

### Step 1

Into a 10 mL reaction vial containing a solution of tert-butyl (3R,5R)-3-[6-(2-cyano-3,6-difluoro-phenoxy)-4-oxo-quinazolin-3-yl]-1-oxa-7-azaspiro[4.4]nonane-7-carboxylate (1, 400.00 mg, 762.61 mol) in N,N-dimethylformamide (5 mL) were added Cs₂CO₃ (496.95 mg, 1.53 mmol) and [methyl(sulfamoyl)amino]ethane (2, 105.38 mg, 762.61 mol) at room temperature and stirred at 65° C. for 16 hours. After completion, the reaction mixture was quenched with water (50 mL) and extracted with ethyl acetate (2×50 mL). The combined organic layers were dried over anhydrous Na₂SO₄, filtered and concentrated under reduced pressure to afford tert-butyl (3R,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (3, 400 mg, 368.45 mol, 48% yield) as a yellow gummy liquid. UPLC-MS (ES−): m/z 641.0 [M−H]⁻.

### Step 2

Into a 25 mL single neck round bottom flask containing a stirred solution of tert-butyl (3R,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (3, 400.00 mg, 622.38 mol) in dichloromethane (5 mL) was added trifluoroacetic acid (6.22 mmol, 476.26 L) at 0° C. and stirred at room temperature for 3 hours. After completion, solvents were concentrated under reduced to get crude, which was triturated with methyl tert-butyl ether (2×2 mL) and dried under vacuum to afford to afford (3R,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-79, 400 mg, 395.98 mol, 64% yield, TFA salt) as a yellow gummy liquid. UPLC-MS (ES+): m/z 543.4 [M+H]⁺.

### Synthesis A80: Synthesis of (3R,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-80)

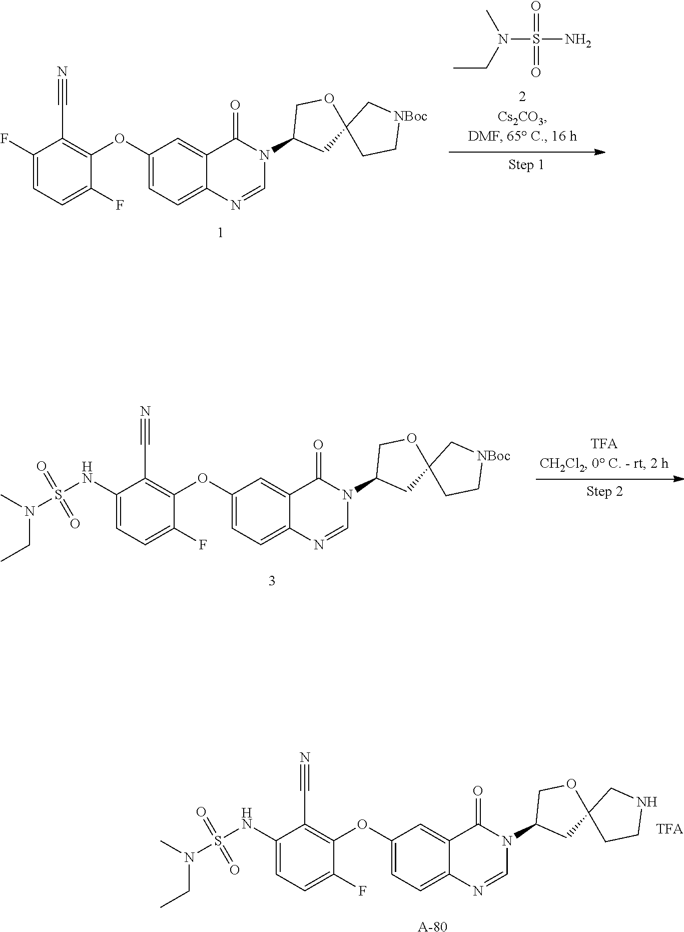

### Step 1

Into a 10 mL reaction vial containing a stirred solution of tert-butyl (3R,5S)-3-[6-(2-cyano-3,6-difluoro-phenoxy)-4-oxo-quinazolin-3-yl]-1-oxa-7-azaspiro[4.4]nonane-7-carboxylate (1, 400.00 mg, 762.61 mol) in N,N-dimethylformamide (5 mL) were added Cs₂CO₃ (496.95 mg, 1.53 mmol) and [methyl(sulfamoyl)amino]ethane (2, 126.46 mg, 915.13 mol) at room temperature. The reaction was stirred at 65° C. for 16 hours. After completion, reaction mixture was quenched with water (20 mL) and extracted with ethyl acetate (2×30 mL). The combined organic layers were dried over anhydrous Na₂SO₄, filtered and filtrates were concentrated under reduced pressure to afford tert-butyl (3R,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (3, 400 mg, 339.20 mol, 44% yield) as a yellow gummy liquid. UPLC-MS (ES−): m/z 641.2 [M−H]⁻.

### Step 2

Into a 25 mL single neck round bottom flask containing a stirred solution of tert-butyl (3R,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (3, 400.00 mg, 622.38 mol) in dichloromethane (10 mL) was added trifluoroacetic acid (6.22 mmol, 476.26 μL) at 0° C. and stirred at room temperature for 2 hours. After completion, solvents were concentrated under residue pressure to get crude, which was triturated with methyl tert-butyl ether (2×4 mL) and dried under vacuum to afford pressure to afford (3R,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-80, 400 mg, 379.53 μmol, 61% yield, TFA salt) as a yellow gummy liquid. UPLC-MS (ES+): m/z 543.3 [M+H]⁺.

### Synthesis A81: Synthesis of (3S,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-81)

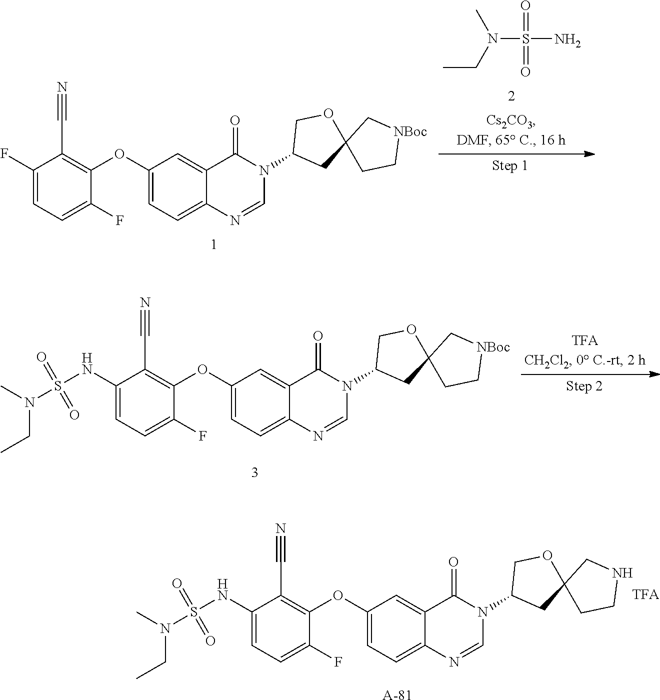

### Step 1

Into a 10 mL reaction vial containing a solution of tert-butyl (3S,5R)-3-[6-(2-cyano-3,6-difluoro-phenoxy)-4-oxo-quinazolin-3-yl]-1-oxa-7-azaspiro[4.4]nonane-7-carboxylate (1, 400.00 mg, 762.61 mol) in N,N-dimethylformamide (5 mL) were added Cs₂CO₃ (496.95 mg, 1.53 mmol) and [methyl(sulfamoyl)amino]ethane (2, 126.46 mg, 915.13 mol) at room temperature. The reaction was stirred at 65° C. for 16 hours. After completion, the reaction mixture was quenched with water (20 mL) and extracted with ethyl acetate (2×20 mL). The combined organic layers were dried over anhydrous Na₂SO₄, filtered and filtrates were concentrated under reduced pressure to afford tert-butyl (3S,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (3, 400 mg, 354.13 mol, 46% yield) as a yellow gummy liquid. UPLC-MS (ES+): m/z 641.2 [M+H]⁺.

### Step 2

Into a 25 mL single neck round bottom flask containing a stirred solution of tert-butyl (3S,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (3, 400.00 mg, 622.38 mol) in dichloromethane (10 mL) was added trifluoroacetic acid (6.22 mmol, 476.26 L) at 0° C. The reaction was stirred at room temperature for 2 hours. After completion, solvents were concentrated to get crude residue, which was triturated with methyl tert-butyl ether (2×4 mL) and dried under vacuum to afford to afford (3S,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-81, 400 mg, 407.55 mol, 65% yield, TFA salt) as a yellow gummy liquid. UPLC-MS (ES+): m/z 543.3 [M+H]⁺.

### Synthesis A82: Synthesis of (3S,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-82)

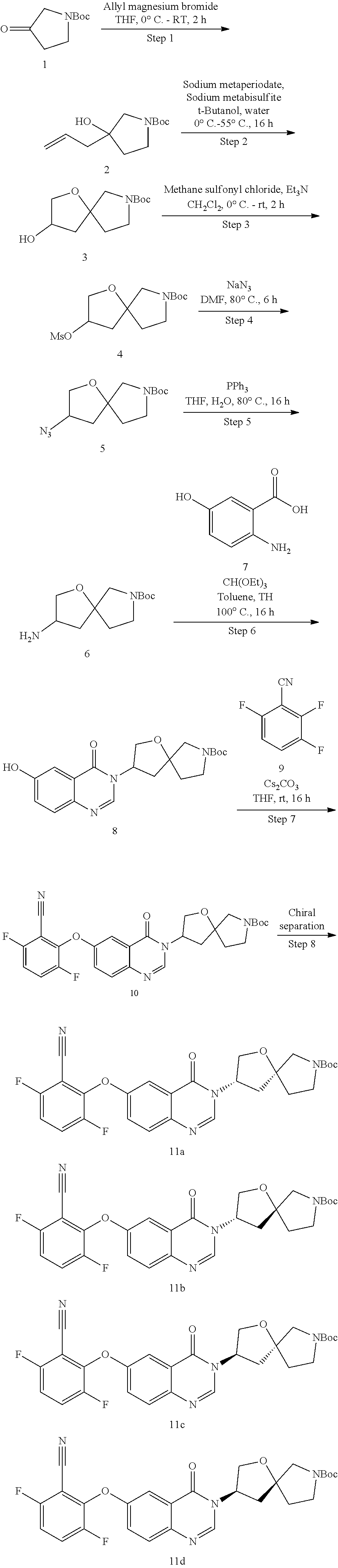

### Step 1

Into a 2L three-neck round bottom flask containing a stirred solution of tert-butyl 3-oxopyrrolidine-1-carboxylate (1, 40 g, 215.96 mmol) in THF (600 mL) was added allyl(bromo)magnesium (1 M, 431.92 mL) dropwise at 0° C. and stirred at room temperature for 2 hours. After completion, the reaction mixture was cooled to 0° C., quenched with aq. NH₄C₁ (400 mL) and extracted with ethyl acetate (2×400 mL). The combined organic layers were washed with saturated NaCl (100 mL), dried over anhydrous Na₂SO₄, filtered and the filtrate was concentrated under reduced pressure to get crude residue which was purified by flash column chromatography (silica gel 230-400 mesh) using 0-100% ethyl acetate/hexane as an eluent while desired product eluted at 20-30% of mobile phase to afford tert-butyl 3-allyl-3-hydroxy-pyrrolidine-1-carboxylate (2, 25 g, 98.99 mmol, 46% yield) as a yellow liquid. ¹H NMR (400 MHz, DMSO-d₆): δ 5.92-5.85 (m, 1H), 5.10-5.04 (m, 2H), 4.76 (d, J=3.20 Hz, 1H), 3.33-3.24 (m, 2H), 3.15-3.00 (m, 2H), 2.29 (d, J=6.8 Hz, 2H), 1.80-1.65 (m, 2H), 1.36 (s, 9H).

### Step 2

Into a 1 L two-neck round bottom flask containing a stirred solution of tert-butyl 3-allyl-3-hydroxy-pyrrolidine-1-carboxylate (2, 25 g, 109.99 mmol) in t-Butanol (250 mL) and water (100 mL) was added sodium metaperiodate (116.88 mmol, 6.47 mL) at 0° C., followed by dropwise addition of a solution of sodium metabisulfite (25.00 g, 131.50 mmol, 16.89 mL) in water (25 mL) at the same temperature. The reaction was stirred at 55° C. for 16 hours. After completion, the reaction mixture was quenched with water (100 mL) and extracted with ethyl acetate (2×100 mL). The combined organic layers were washed with saturated NaCl (100 mL), dried over anhydrous Na₂SO₄, filtered and the filtrate was concentrated under reduced pressure to get the crude residue which was purified by flash column chromatography (silica gel 230-400 mesh) using 0-100% ethyl acetate/hexane as an eluent while desired product was eluted at 70-80% of mobile phase to afford tert-butyl 3-hydroxy-1-oxa-7-azaspiro[4.4]nonane-7-carboxylate (3, 10 g, 28.77 mmol, 26% yield) as a black gummy liquid. ¹H NMR (400 MHz, DMSO-d₆): δ 4.35-4.25 (m, 1H), 3.80-3.76 (m, 1H), 3.59-3.56 (m, 1H), 3.35-3.18 (m, 5H), 2.10-2.00 (m, 2H), 1.85-1.75 (m, 2H), 1.40 (s, 9H).

### Step 3

Into a 500 mL two-neck round bottom flask containing a stirred solution of tert-butyl 3-hydroxy-1-oxa-7-azaspiro[4.4]nonane-7-carboxylate (3, 10 g, 41.10 mmol) in dichloromethane (200 mL) were added triethylamine (123.31 mmol, 17.19 mL) and methanesulfonyl chloride (61.65 mmol, 4.78 mL) dropwise at 0° C. The reaction was stirred at room temperature for 2 hours. After completion, the reaction mixture was quenched with water (100 mL) and extracted with dichloromethane (2×100 mL). The combined organic layers were washed with aq. NaHCO₃ (100 mL), saturated NaCl (100 mL), dried over anhydrous Na₂SO₄, filtered and concentrated under reduced pressure to afford tert-butyl 3-methylsulfonyloxy-1-oxa-7-azaspiro[4.4]nonane-7-carboxylate (4, 10 g, 29.87 mmol, 73% yield) as a black gummy liquid. UPLC-MS (ES+): m/z 222.2 [M−Boc+H]⁺.

### Step 4

Into a 500 mL two-neck round bottom flask containing a stirred solution of tert-butyl 3-methylsulfonyloxy-1-oxa-7-azaspiro[4.4]nonane-7-carboxylate (4, 10 g, 31.11 mmol) in N,N-dimethylformamide (200 mL) was added sodium azide (3.03 g, 46.67 mmol, 1.64 mL) at room temperature. The reaction was stirred at 80° C. for 6 hours. After completion, the reaction mixture was cooled to room temperature, quenched with water (100 mL) and extracted with ethyl acetate (2×100 mL). The combined organic layers were dried over anhydrous Na₂SO₄, filtered and filtrate was concentrated under reduced pressure to get crude residue which was purified by flash column chromatography (silica gel 230-400 mesh) using 0-100% ethyl acetate/hexane as an eluent while desired product eluted at 40-50% of mobile phase to afford tert-butyl 3-azido-1-oxa-7-azaspiro[4.4]nonane-7-carboxylate (5, 10 g, 22.36 mmol, 72% yield) as a brown gummy liquid. ¹H NMR (400 MHz, DMSO-d₆): δ 4.45-4.30 (m, 1H), 3.95-3.85 (m, 1H), 3.85-3.70 (m, 1H), 3.28-3.00 (m, 3H), 2.40-2.20 (m, 1H), 2.10-1.80 (m, 3H), 1.40 (s, 9H).

### Step 5

Into a 250 mL two neck round bottom flask containing a solution of tert-butyl 3-azido-1-oxa-7-azaspiro[4.4]nonane-7-carboxylate (5, 15 g, 55.91 mmol) in THF (300 mL) and water (75 mL) was added triphenylphosphine (23.02 g, 87.77 mmol) at room temperature. The reaction was stirred at 80° C. for 16 hours. After completion, the reaction mixture was cooled to room temperature, quenched with water (500 mL) and extracted with ethyl acetate (2×500 mL). The combined organic layers were dried over anhydrous Na₂SO₄, filtered and concentrated under reduced pressure to get crude residue which was purified by flash column chromatography (silica gel 230-400 mesh) using 0-20% methanol/dichloromethane as an eluent while desired product eluted at 15-20% of mobile phase to afford tert-butyl 3-amino-1-oxa-7-azaspiro[4.4]nonane-7-carboxylate (6, 2 g, 6.60 mmol, 12% yield) as a brown gummy liquid. ¹H NMR (400 MHz, DMSO-d₆): δ 3.83-3.79 (m, 1H), 3.53-3.51 (m, 1H), 3.50-3.23 (m, 8H), 2.00-1.95 (m, 1H), 1.61-1.55 (m, 2H), 1.41 (s, 9H).

### Step 6

Into a 100 mL sealed tube containing a solution of tert-butyl 3-amino-1-oxa-7-azaspiro[4.4]nonane-7-carboxylate (6, 2 g, 8.25 mmol) in toluene (28 mL) and THF (12 mL) were added 2-amino-5-hydroxy-benzoic acid (7, 1.26 g, 8.25 mmol) and triethyl orthoformate (22.29 mmol, 3.71 mL) at room temperature. The reaction mixture was stirred at 100° C. for 16 hours. After completion, the reaction mixture was quenched with water (50 mL) and extracted with ethyl acetate (2×50 mL). The combined organic layers were dried over anhydrous Na₂SO₄, filtered and concentrated under reduced pressure to afford crude residue which was purified by flash column chromatography (silica gel 230-400 mesh) using 0-100% ethyl acetate/hexane as an eluent while desired product eluted at 70-80% of mobile phase to afford tert-butyl 3-(6-hydroxy-4-oxo-quinazolin-3-yl)-1-oxa-7-azaspiro[4.4]nonane-7-carboxylate (8, 2 g, 3.77 mmol, 46% yield) as an off-white solid. UPLC-MS (ES+): m/z 388.4 [M+H]⁺.

### Step 7

Into a 250 mL single neck round bottom flask containing a stirred solution of tert-butyl 3-(6-hydroxy-4-oxo-quinazolin-3-yl)-1-oxa-7-azaspiro[4.4]nonane-7-carboxylate (8, 2 g, 5.16 mmol) in THF (40 mL) were added 2,3,6-trifluorobenzonitrile (9, 6.19 mmol, 715.54 μL) and Cs₂CO₃ (3.36 g, 10.32 mmol) at room temperature. The reaction mixture was stirred at the same temperature for 16 hours. After completion, the reaction mixture was filtered through celite pad and washed with ethyl acetate (50 mL). The filtrate was concentrated under reduced pressure to get crude residue which was purified by flash column chromatography (silica gel 230-400 mesh) using 0-100% ethyl acetate/hexane as an eluent while desired product eluted at 60-70% of mobile phase to afford tert-butyl 3-[6-(2-cyano-3,6-difluoro-phenoxy)-4-oxo-quinazolin-3-yl]-1-oxa-7-azaspiro[4.4]nonane-7-carboxylate (10, 2.5 g, 4.53 mmol, 88% yield) as an off-white solid. UPLC-MS (ES+): m/z 525.1 [M+H]⁺.

### Step 8

The tert-butyl 3-[6-(2-cyano-3,6-difluoro-phenoxy)-4-oxo-quinazolin-3-yl]-1-oxa-7-azaspiro[4.4]nonane-7-carboxylate (10, 2.5 g, 4.77 mmol) which was a mixture of 4-isomers were separated by SFC purification.

### Method of Analysis for chiral SFC separation

Instrument: PIC-22-016
Column: LUXA1-(250*30) mm, 5 m
Mobile Phase: CO₂: 0.2% FA in IPA: CAN (1:1) [70:30]
Total Flow: 100 mL/min
Back pressure: 100 bar
Wavelength: 220 nm
Cycle time: 12 min

Peak-1: First eluting compound (Peak-1) was collected and concentrated under reduced pressure to afford tert-butyl (3S,5S)-3-[6-(2-cyano-3,6-difluoro-phenoxy)-4-oxo-quinazolin-3-yl]-1-oxa-7-azaspiro[4.4]nonane-7-carboxylate (11a, 400 mg, 706.18 mol, 15% yield) as a yellow gummy liquid. UPLC-MS (ES−): m/z 523.2 [M−H]⁻.

Peak-2 & Peak-3: Second eluting compound (mixture of Peak-2 & Peak-3) was collected and concentrated under reduced pressure to afford 1 g of yellow gummy liquid (mixture of 2-isomers).

Peak-4: Last eluting compound (Peak-4) was collected and concentrated to afford tert-butyl (3R,5R)-3-[6-(2-cyano-3,6-difluoro-phenoxy)-4-oxo-quinazolin-3-yl]-1-oxa-7-azaspiro[4.4]nonane-7-carboxylate (11d, 400 mg, 745.83 mol, 16% yield) as a yellow gummy liquid. UPLC-MS (ES−): m/z 523.2 [M−H]⁻.

The above second eluting fraction which was a mixture of two isomers (Peak-2 & Peak-3) were separated by Chiral SFC.

### Method of Analysis for chiral SFC separation

Instrument: PIC-22-016
Column: I-Cellulose-B (250*30) mm, 5 m
Mobile Phase: CO₂: 0.2% FA in IPA: CAN (1:1) [75:25]
Total Flow: 100 mL/min
Back pressure: 120 bar
Wavelength: 220 nm
Cycle time: 3.5 min

Peak-2: First eluting compound (Peak-2) was collected and concentrated under reduced pressure to afford tert-butyl (3S,5R)-3-[6-(2-cyano-3,6-difluoro-phenoxy)-4-oxo-quinazolin-3-yl]-1-oxa-7-azaspiro[4.4]nonane-7-carboxylate (11b, 400 mg, 752.70 mol, 16% yield) as a yellow gummy liquid. UPLC-MS (ES−): m/z 523.3 [M−H]⁻.

Peak-3: Second eluting compound (Peak-3) was collected and concentrated under reduced pressure to afford tert-butyl (3R,5S)-3-[6-(2-cyano-3,6-difluoro-phenoxy)-4-oxo-quinazolin-3-yl]-1-oxa-7-azaspiro[4.4]nonane-7-carboxylate (11c, 400 mg, 761.08 mol, 16% yield) as a yellow gummy liquid. UPLC-MS (ES−): m/z 523.2 [M−H]⁻.

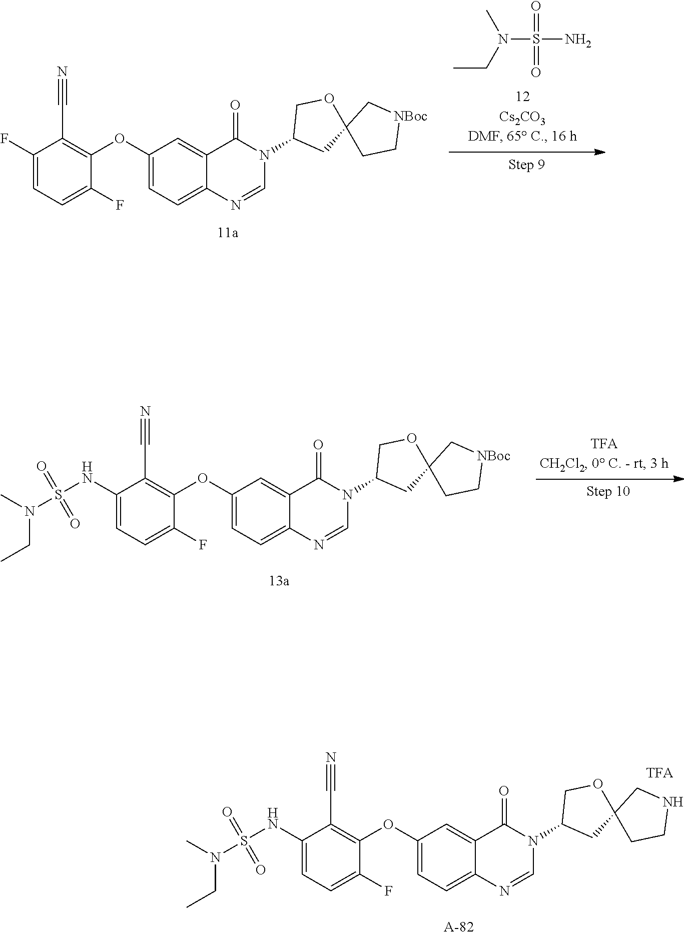

### Step 9

Into a 10 mL reaction vial containing a solution of tert-butyl (3S,5S)-3-[6-(2-cyano-3,6-difluoro-phenoxy)-4-oxo-quinazolin-3-yl]-1-oxa-7-azaspiro[4.4]nonane-7-carboxylate (11a, 400.00 mg, 762.61 mol) in N,N-dimethylformamide (5 mL) were added Cs₂CO₃ (496.95 mg, 1.53 mmol) and [methyl(sulfamoyl)amino]ethane (12, 105.38 mg, 762.61 mol) at room temperature. The reaction was stirred at 65° C. for 16 hours. After completion, the reaction mixture was quenched with water (50 mL) and extracted with ethyl acetate (2×50 mL). The combined organic layers were dried over anhydrous Na₂SO₄, filtered and concentrated under reduced pressure to afford tert-butyl (3S,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (13a, 400 mg, 402.68 mol, 53% yield) as a yellow gummy liquid. UPLC-MS (ES+): m/z 643.2 [M+H]⁺.

### Step 10

Into a 25 mL single neck round bottom flask containing a solution of tert-butyl (3S,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane-7-carboxylate (13a, 400.00 mg, 622.38 mol) in dichloromethane (5 mL) was added trifluoroacetic acid (6.22 mmol, 476.26 L) at 0° C. The reaction was stirred at room temperature for 3 hours. After completion, solvents were concentrated under reduced pressure to get crude, which was triturated with methyl tert-butyl ether(10 mL) and dried well to afford (3S,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-82, 400 mg, 389.88 mol, 63% yield, TFA salt) as a yellow gummy liquid. UPLC-MS (ES+): m/z 543.3 [M+H]⁺.

### Synthesis A83: Synthesis of 2-(2-azaspiro[3.3]heptan-6-yl)-7-[2-cyano-3-[[ethyl(methyl)sulfamoyl]amino]-6-fluoro-phenoxy]quinoxaline (A-83)

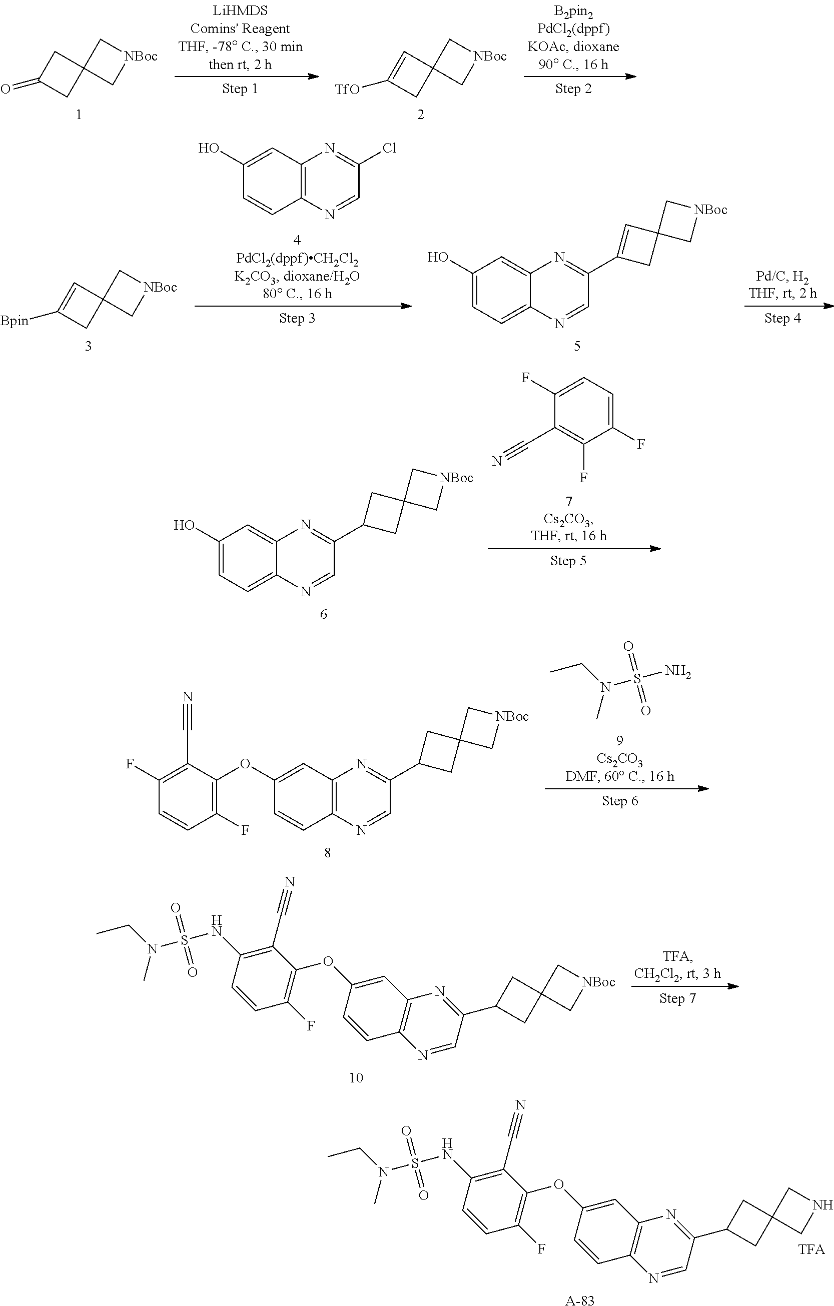

### Step 1

Into a 1L three-neck round bottom flask containing a well-stirred solution of tert-butyl 6-oxo-2-azaspiro[3.3]heptane-2-carboxylate (1, 10 g, 47.34 mmol) in anhydrous THF (150 mL) was added LiHMDS (1.0 M in THF) (1.0 M, 94.67 mL) dropwise at −78° C. under nitrogen atmosphere and the mixture was stirred at same temperature for 1 hour. A solution of N-(5-chloro-2-pyridyl)-1,1,1-trifluoro-N-(trifluoromethylsulfonyl)methanesulfonamide (27.88 g, 71.00 mmol) in THF (50 mL) was added drop wise to the above reaction mixture at −78° C., after addition the reaction mixture was allowed to room temperature and stirred for 2 hours. After completion of the reaction, the reaction mixture was quenched with saturated NH₄C₁ solution (100 mL) and extracted with ethyl acetate (2×100 mL). The combined organic layers were washed with saturated NaCl solution (500 mL), dried over anhydrous Na₂SO₄, filtered and concentrated under reduced pressure to get a crude residue, which was purified by flash column chromatography (250 g, 230-400 silica-gel mesh) with 0-100% ethyl acetate/hexane to afford tert-butyl 6-(trifluoromethylsulfonyloxy)-2-azaspiro[3.3]hept-6-ene-2-carboxylate (2, 8 g, 23.28 mmol, 49% yield) as an off-white solid. ELSD-MS (ES+): m/z 244.0 [M−Boc+H]⁺.

### Step 2

Into a 250 mL single neck round bottom flask containing a well-stirred solution of tert-butyl 6-(trifluoromethylsulfonyloxy)-2-azaspiro[3.3]hept-6-ene-2-carboxylate (2, 6 g, 17.46 mmol) and 4,4,5,5-tetramethyl-2-(4,4,5,5-tetramethyl-1,3,2-dioxaborolan-2-yl)-1,3,2-dioxaborolane (5.76 g, 22.70 mmol) in anhydrous 1,4 dioxane (100 mL) was added KOAc (4.28 g, 43.65 mmol) at room temperature under nitrogen atmosphere and the resulting mixture was degassed by bubbling nitrogen gas for 10 minutes. Subsequently, bis(1-diphenylphosphanylcyclopenta-2,4-dien-1-yl)iron dichloropalladium dichloromethane (1.43 g, 1.75 mmol) was added to the reaction mixture and stirred at 90° C. for 16 hours. After completion of the reaction, the reaction mixture was cooled to room temperature and filtered through a pad of celite and washed with ethyl acetate (150 mL). The filtrate was concentrated under reduced pressure to get a crude residue, which was purified by flash column chromatography (100 g, neutral alumina) with 0-100% ethyl acetate/hexane to afford tert-butyl 6-(4,4,5,5-tetramethyl-1,3,2-dioxaborolan-2-yl)-2-azaspiro[3.3]hept-6-ene-2-carboxylate (3, 1.5 g, 1.98 mmol, 11% yield) as an off-white solid. ELSD-MS (ES+): m/z 222.2 [M−Boc+H]⁺.

### Step 3

Into a 100 mL single neck round bottom flask containing a well stirred solution of 3-chloroquinoxalin-6-ol (3, 700 mg, 3.52 mmol) in 1,4-dioxane (20 mL) and water (5 mL) were added tert-butyl 6-(4,4,5,5-tetramethyl-1,3,2-dioxaborolan-2-yl)-2-azaspiro[3.3]hept-6-ene-2-carboxylate (4, 1.47 g, 4.58 mmol) and K2CO₃, anhydrous, 99% (1.46 g, 10.57 mmol) at room temperature under nitrogen atmosphere. The resulting mixture was purged with nitrogen gas for 10 min. Subsequently, bis(1-diphenylphosphanylcyclopenta-2,4-dien-1-yl)iron dichloropalladium dichloromethane (287.74 mg, 352.34 mol) was added to the reaction mixture and stirred at 80° C. for 16 hours. After completion of the reaction, the reaction mixture was allowed to room temperature and filtered over celite bed and washed with ethyl acetate (50 mL). The filtrate was concentrated under reduced pressure to get a crude residue, which was purified by column chromatography (50 g, 230-400 silicagel mesh) with 0-100% ethyl acetate in hexane, while desired product was eluted at 50% of mobile phase to afford tert-butyl 6-(7-hydroxyquinoxalin-2-yl)-2-azaspiro[3.3]hept-6-ene-2-carboxylate (5, 750 mg, 2.03 mmol, 58% yield) as a pale yellow solid. UPLC-MS (ES+): m/z 340.1 [M+H]⁺.

### Step 4

Into a 100 mL single neck round bottom flask containing a well stirred solution of tert-butyl 6-(7-hydroxyquinoxalin-2-yl)-2-azaspiro[3.3]hept-6-ene-2-carboxylate (5, 1.1 g, 3.24 mmol) in THF (40 mL) was added palladium 10% on carbon (50% wet) (413.91 mg, 1.94 mmol) at room temperature under nitrogen atmosphere. The resulting mixture was stirred under hydrogen pressure (bladder pressure) at room temperature for 2 hours. After completion of the reaction, the reaction mixture was filtered over celite bed and washed with ethyl acetate (100 mL). The filtrate was concentrated under reduced pressure to afford tert-butyl 6-(7-hydroxyquinoxalin-2-yl)-2-azaspiro[3.3]heptane-2-carboxylate (6, 1 g, 2.58 mmol, 79% yield) as a pale brown gum. UPLC-MS (ES+): m/z 342.3 [M+H]⁺.

### Step 5

Into a 30 mL glass vial containing a well stirred solution of tert-butyl 6-(7-hydroxyquinoxalin-2-yl)-2-azaspiro[3.3]heptane-2-carboxylate (6, 1 g, 2.58 mmol) in THF (20 mL) were added Cs₂CO₃ (2.10 g, 6.44 mmol) and 2,3,6-trifluorobenzonitrile (7, 3.09 mmol, 356.96 L) at room temperature under nitrogen atmosphere. The reaction mixture was stirred at room temperature for 16 hours. After completion of the reaction, the reaction mixture was filtered and concentrated under reduced pressure to get a crude residue, which was purified by flash column chromatography (50 g, 230-400 silicagel mesh) with 0-100% ethyl acetate in hexane, while pure compound was eluted at 50% of mobile phase to afford tert-butyl 6-[7-(2-cyano-3,6-difluoro-phenoxy)quinoxalin-2-yl]-2-azaspiro[3.3]heptane-2-carboxylate (8, 1 g, 2.01 mmol, 78% yield) as a pale yellow solid. UPLC-MS (ES+): m/z 479.3 [M+H]⁺.

### Step 6

Into a 100 mL single neck round bottom flask containing a stirred solution of tert-butyl 6-[7-(2-cyano-3,6-difluoro-phenoxy)quinoxalin-2-yl]-2-azaspiro[3.3]heptane-2-carboxylate (8, 1 g, 2.09 mmol) in anhydrous N,N-dimethylformamide (15 mL) were added Cs₂CO₃(1.70 g, 5.22 mmol) and [methyl(sulfamoyl)amino]ethane (9, 433.20 mg, 3.13 mmol) at room temperature under nitrogen atmosphere. The reaction mixture was stirred at 60° C. for 16 hours. After completion of the reaction, the reaction mixture was diluted with water (50 mL), the solid precipitated was filtered through sintered funnel and washed with water. The obtained filtrate was extracted with ethyl acetate (2×100 mL) and washed with saturated NaCl solution (100 mL). The combined organic layers were dried over Na₂SO₄, concentrated under reduced pressure to get a crude residue, which was purified by flash column chromatography (50 g, 230-400 silica-gel mesh) with 0-100% ethyl acetate in hexane, while pure compound was eluted at 60% of mobile phase to afford tert-butyl 6-[7-[2-cyano-3-[[ethyl(methyl)sulfamoyl]amino]-6-fluoro-phenoxy]quinoxalin-2-yl]-2-azaspiro[3.3]heptane-2-carboxylate (10, 520 mg, 629.75 mol, 30% yield) as a pale yellow solid. UPLC-MS (ES+): m/z 597.3 [M+H]⁺.

### Step 7

Into a 50 mL single neck round bottom flask containing a well stirred solution of tert-butyl 6-[7-[2-cyano-3-[[ethyl(methyl)sulfamoyl]amino]-6-fluoro-phenoxy]quinoxalin-2-yl]-2-azaspiro[3.3]heptane-2-carboxylate (10, 500 mg, 605.53 mol) in dichloromethane (15 mL) was added trifluoroacetic acid (13.07 mmol, 1 mL) at 0° C. under nitrogen atmosphere. The reaction mixture was stirred at room temperature for 3 hours. After completion of the reaction, solvents were concentrated under reduced pressure to get a crude residue, which was triturated with methyl tert-butyl ether (2×5 mL) and dried under vacuum to afford 2-(2-azaspiro[3.3]heptan-6-yl)-7-[2-cyano-3-[[ethyl(methyl)sulfamoyl]amino]-6-fluoro-phenoxy]quinoxaline (A-83, 480 mg, 601.08 mol, 99% yield, TFA salt) as a pale yellow gummy-solid. UPLC-MS (ES+): m/z 497.3 [M+H]⁺.

### Synthesis A84: Synthesis of 2-(2-azaspiro[3.3]heptan-6-yl)-7-[2-cyano-6-fluoro-3-[[2-hydroxyethyl(methyl)sulfamoyl]amino]phenoxy]quinoxaline (A-84)

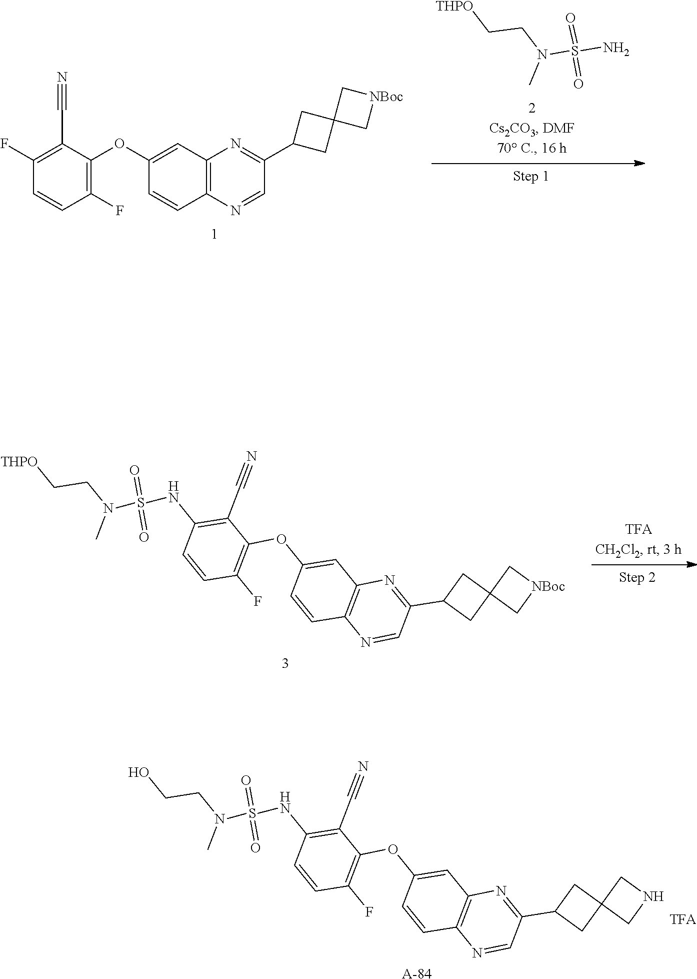

### Step 1

Into a 30 mL glass vial containing a well stirred solution of tert-butyl 6-[7-(2-cyano-3,6-difluoro-phenoxy)quinoxalin-2-yl]-2-azaspiro[3.3]heptane-2-carboxylate (1, 1 g, 2.00 mmol) in N,N-dimethylformamide (10 mL) were added cesium carbonate (1.95 g, 5.99 mmol) and 2-[2-[methyl(sulfamoyl)amino]ethoxy]tetrahydropyran (2, 1.43 g, 5.99 mmol) at room temperature under nitrogen atmosphere. The reaction mixture was stirred at 70° C. for 16 hours. After completion of the reaction, the reaction mixture was filtered and the filtrate was diluted with ethyl acetate (30 mL) and washed with ice cold water (2×30 mL), saturated NaCl (20 mL), dried over anhydrous sodium sulphate and concentrated under reduced pressure to get a crude residue, which was purified by flash column chromatography (50 g, 230-400 silicagel mesh) with 0-100% ethyl acetate/hexane, while pure compound was eluted at 60% of ethyl acetate/hexane to afford tert-butyl 6-[7-[2-cyano-6-fluoro-3-[[methyl(2-tetrahydropyran-2-yloxyethyl)sulfamoyl]amino]phenoxy]quinoxalin-2-yl]-2-azaspiro[3.3]heptane-2-carboxylate (3, 600 mg, 696.02 mol, 35% yield) as a pale yellow solid. UPLC-MS (ES+): m/z 697.2 [M+H]⁺.

### Step 2

Into a 50 mL single neck round bottom flask containing a well stirred solution of tert-butyl 6-[7-[2-cyano-6-fluoro-3-[[methyl(2-tetrahydropyran-2-yloxyethyl)sulfamoyl]amino]phenoxy]quinoxalin-2-yl]-2-azaspiro[3.3]heptane-2-carboxylate (3, 540 mg, 626.42 mol) in dichloromethane (15 mL) was added trifluoroacetic acid (13.07 mmol, 1 mL) at 0° C. under nitrogen atmosphere. The reaction mixture was stirred at room temperature for 3 hours. After completion of the reaction, the reaction mixture was concentrated under reduced pressure to get the crude residue which was triturated with methyl tert-butyl ether (3 mL) and dried under vacuum to afford 2-(2-azaspiro[3.3]heptan-6-yl)-7-[2-cyano-6-fluoro-3-[[2-hydroxyethyl(methyl)sulfamoyl]amino]phenoxy]quinoxaline (A-84, 450 mg, 523.41 mol, 84% yield, TFA salt) as a pale yellow gummy-solid. UPLC-MS (ESI): m/z 513.2 [M+H]⁺.

### Example 2: Synthesis of CRBN Binders

### Synthesis B1: Synthesis of 1-(6-bromo-5-fluoro-1-methyl-indazol-3-yl)hexahydropyrimidine-2,4-dione (B-1)

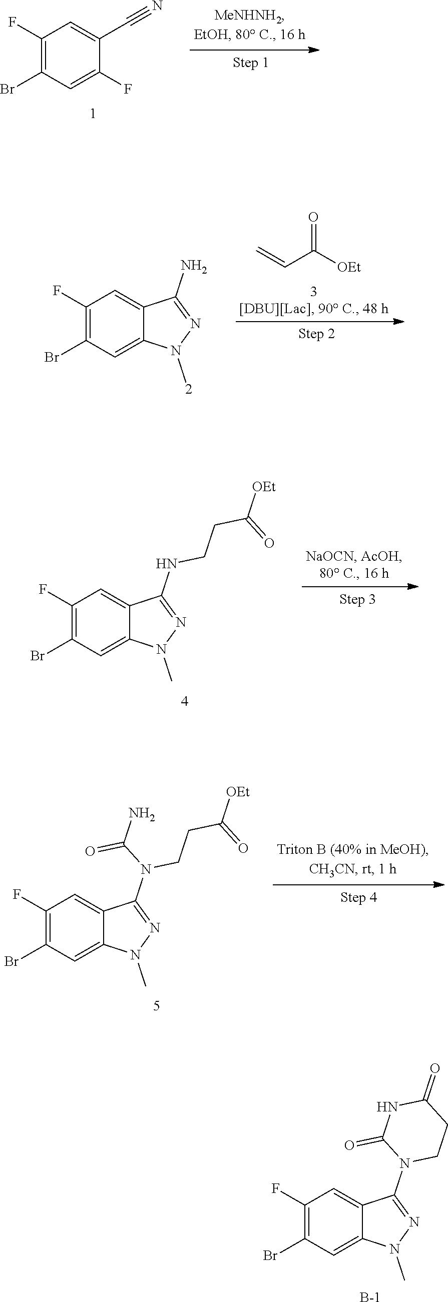

### Step 1

To a suspension of 4-bromo-2,5-difluoro-benzonitrile (1; 100 g, 458.72 mmol) in EtOH (450 mL), was added methylhydrazine (85% solution in water, 113.0 mL, 1.83 mol) at room temperature. The resulting mixture was heated at 80° C. for 16 hours. The reaction mixture was cooled to 0° C., treated with water (500 mL), and stirred for 30 min. The solid precipitated was filtered, washed with water (250 mL) followed by petroleum ether (200 mL) and dried under vacuum to afford 6-bromo-5-fluoro-1-methyl-indazol-3-amine (2; 50 g, 204.48 mmol, 45% yield) as a pale-yellow solid. LC-MS (ES+): m/z 244.0 [M+H]⁺.

### Step 2

Into a four-neck round bottom flask was charged with 1,8-diazabicyclo[5.4.0]undec-7-ene (73.4 mL, 490.74 mmol), cooled to 0° C., was added DL-lactic acid (44.20 g, 490.74 mmol). The resulting mixture was stirred at room temperature for 18 h before 6-bromo-5-fluoro-1-methyl-indazol-3-amine (2; 60 g, 245.37 mmol) and ethyl prop-2-enoate (3; 186.1 mL, 1.72 mol) were added to the mixture. The resulting mixture was heated at 90° C. for 48 hours. The reaction mixture was cooled to 0° C., treated with water (500 mL), and extracted with ethyl acetate (2×500 mL). The combined organic phases were washed with saturated NaCl solution (100 mL) and dried over anhydrous Na₂SO₄. The solution was filtered and concentrated under reduced pressure to give a crude product, which was purified by flash chromatography (Silica gel, 60-120 mesh) eluted with 0-100% ethyl acetate in petroleum ether, while the desired product was eluted at 20-25% ethyl acetate in petroleum ether. The fractions containing the product were pooled and concentrated under reduced pressure to afford ethyl 3-[(6-bromo-5-fluoro-1-methyl-indazol-3-yl)amino]propanoate (4; 55 g, 148.18 mmol, 60% yield) as a yellow solid. LC-MS (ES*): m/z 344.0 [M+H]⁺.

### Step 3

To a suspension of ethyl 3-[(6-bromo-5-fluoro-1-methyl-indazol-3-yl)amino]propanoate (4; 55 g, 148.18 mmol) in AcOH (400 mL), was added sodium cyanate (28.90 g, 444.55 mmol) at room temperature. The resulting mixture was heated at 80° C. for 16 hours. The reaction was brought to room temperature, concentrated under reduced pressure to give a residue, which was cooled to 0° C., treated with 10% aqueous NaHCO₃ solution (200 mL), and extracted with dichloromethane (2×500 mL). The combined organic phases were washed with 10% aqueous NaHCO₃ solution (200 mL) and dried over anhydrous Na₂SO₄. The solution was filtered and concentrated under reduced pressure to give a crude product, which was triturated with methyl tert-butyl ether (60 mL) and dried under vacuum to afford ethyl 3-[(6-bromo-5-fluoro-1-methyl-indazol-3-yl)-carbamoyl-amino]propanoate (5; 35 g, 85.31 mmol, 58% yield) as an off-white solid. LC-MS (ES+): m/z 389.0 [M+H]⁺ (bromo pattern).

### Step 4

To a suspension of ethyl 3-[(6-bromo-5-fluoro-1-methyl-indazol-3-yl)-carbamoyl-amino]propanoate (5; 70 g, 170.62 mmol) in acetonitrile (350 mL), was added benzyltrimethylammonium hydroxide (40 wt. % in methanol; 23.3 mL, 51.19 mmol) at room temperature. The resulting mixture was stirred at room temperature for 1 hour. The reaction mixture was concentrated under reduced pressure and treated with water (100 mL), the solid precipitated was filtered, washed with water (100 mL), and dried under vacuum to afford 1-(6-bromo-5-fluoro-1-methyl-indazol-3-yl)hexahydropyrimidine-2,4-dione (B-1; 50.25 g, 140.57 mmol, 82% yield) as an off-white solid. LC-MS (ES+): m/z 343.0 [M+H]⁺ (bromo pattern). ¹H NMR (400 MHz, DMSO-d₆): δ 10.60 (s, 1H), 8.14 (d, J=5.6 Hz, 1H), 7.61 (d, J=9.2 Hz, 1H), 3.99 (s, 3H), 3.93 (t, J=6.8 Hz, 2H), 2.51 (t, J=2.0 Hz, 2H).

### Synthesis B2: Synthesis of 1-[5-fluoro-1-methyl-6-(4-oxo cyclohexyl)indazol-3-yl]hexahydropyrimidine-2,4-dione (B-2)

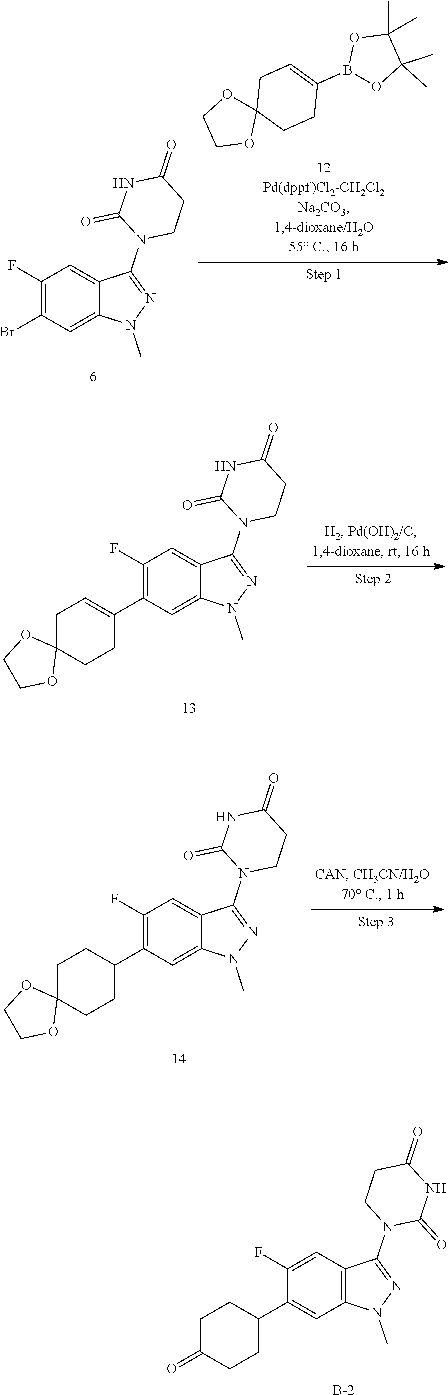

### Step 1

To a solution of 1-(6-bromo-5-fluoro-1-methyl-indazol-3-yl)hexahydropyrimidine-2,4-dione (6; 19 g, 53.36 mmol) and 2-(1,4-dioxaspiro[4.5]dec-7-en-8-yl)-4,4,5,5-tetramethyl-1,3,2-dioxaborolane (12; 18.46 g, 69.37 mmol) in 1,4-dioxane (320 mL) and water (80 mL), was added sodium carbonate (14.14 g, 133.41 mmol) at room temperature. The resulting mixture was degassed with nitrogen for 10 min before bis(diphenylphosphino)ferrocene]palladium(II), complex with dichloromethane (4.35 g, 5.34 mmol) was added to the mixture. The resulting mixture was heated at 55° C. for 16 hours. The reaction mixture was filtered through a pad of celite and washed with ethyl acetate (2×300 mL). The filtrate was treated with water (100 mL) and the organic phase was separated and dried over anhydrous sodium sulfate. The solution was filtered, and the filtrate was concentrated under reduced pressure to give a crude product, which was triturated with acetonitrile (20 mL) and dried to afford 1-[6-(1,4-dioxaspiro[4.5]dec-7-en-8-yl)-5-fluoro-1-methyl-indazol-3-yl]hexahydropyrimidine-2,4-dione (13; 20 g, 49.20 mmol, 92% yield) as a yellow solid. LC-MS (ES*): m/z 401.2 [M+H]⁺.

### Step 2

To a suspension of 1-[6-(1,4-dioxaspiro[4.5]dec-7-en-8-yl)-5-fluoro-1-methyl-indazol-3-yl]hexahydropyrimidine-2,4-dione (13; 18 g, 44.28 mmol) in 1,4-dioxane (360 mL), was added palladium hydroxide on carbon (20 wt. % loading; 18 g, 128.17 mmol) at room temperature. The resulting suspension was stirred at room temperature under a hydrogen atmosphere (~1 kg/cm²) for 16 hours. The reaction mixture was filtered through a pad of celite, and the celite was washed with ethyl acetate/1,4-dioxane (5000 mL). The filtrate was concentrated under reduced pressure to afford 1-[6-(1,4-dioxaspiro[4.5]decan-8-yl)-5-fluoro-1-methyl-indazol-3-yl]hexahydropyrimidine-2,4-dione (14; 18 g, 36.29 mmol, 82% yield) as an off-white solid. LC-MS (ES+): m/z 403.2 [M+H]⁺.

### Step 3

To a suspension of 1,-[6-(1,4-dioxaspiro[4.5]decan-8-yl)-5-fluoro-1-methyl-indazol-3-yl]hexahydropyrimidine-2,4-dione (14; 17 g, 34.28 mmol) in acetonitrile (85 mL) and water (85 mL), was added ceric ammonium nitrate (1.32 g, 2.40 mmol) at room temperature. The resulting mixture was heated at 70° C. for 1 hour. The reaction mixture was brought to room temperature and treated with ice-cold water (300 mL). The solid precipitated was filtered and dried under vacuum to afford 1-[5-fluoro-1-methyl-6-(4-oxo cyclohexyl)indazol-3-yl]hexahydropyrimidine-2,4-dione (B-2; 12.1 g, 32.94 mmol, 96% yield) as an off-white solid. LC-MS (ES+): m/z 359.2 [M+H]⁺. ¹H NMR (400 MHz, DMSO-d₆): δ 10.57 (s, 1H), 7.63 (d, J=6.0 Hz, 1H), 7.41 (d, J=10.8 Hz, 1H), 3.99 (s, 3H), 3.91 (t, J=6.8 Hz, 2H), 3.49-3.42 (m, 1H), 2.77-2.68 (m, 2H), 2.68-2.64 (m, 2H), 2.34-2.30 (m, 2H), 2.15-2.08 (m, 2H), 2.03-1.86 (m, 2H).

### Synthesis B3: Synthesis of 1-[5-fluoro-6-(3-fluoro-4-oxo-cyclohexyl)-1-methyl-indazol-3-yl]hexahydropyrimidine-2,4-dione (B-3)

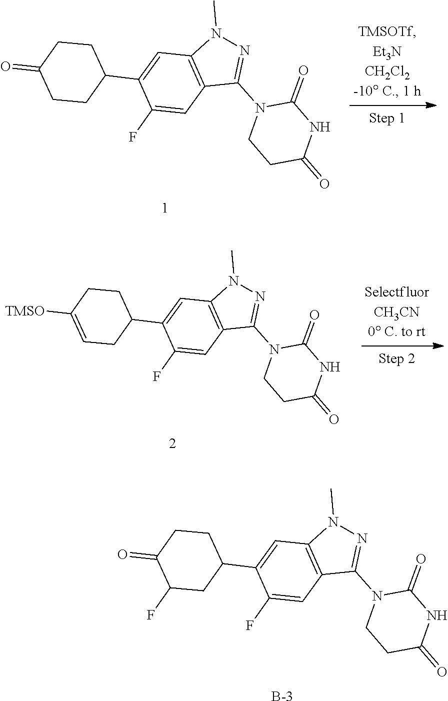

### Step 1

Into a 100 mL two neck round-bottom flask containing a well-stirred solution of 1-[5-fluoro-1-methyl-6-(4-oxocyclohexyl)indazol-3-yl]hexahydropyrimidine-2,4-dione (1, 2.0 g, 5.58 mmol), triethylamine (16.74 mmol, 2.33 mL) in dichloromethane (40 mL), was added trimethylsilyl trifluoromethanesulfonate (7.26 mmol, 1.31 mL) dissolved in dichloromethane (10 mL) at −10° C. under nitrogen atmosphere. The resulting reaction mixture was stirred at room temperature for 1 hour. After completion of the reaction, the reaction mixture was poured into water (50 mL) and extracted with dichloromethane (3×50 mL). The combined organic layer was dried over anhydrous Na₂SO₄, filtered, and concentrated under reduced pressure to afford 1-[5-fluoro-1-methyl-6-(4-trimethylsilyloxycyclohex-3-en-1-yl)indazol-3-yl]hexahydropyrimidine-2,4-dione (2, 2.2 g, 5.11 mmol, 92% yield) as an off-white semi-solid. ¹H NMR (400 MHz, DMSO-d₆): δ 10.56 (s, 1H), 7.59 (d, J=6.00 Hz, 1H), 7.38 (d, J=10.80 Hz, 1H), 4.93 (s, 1H), 4.00 (s, 3H), 3.91 (t, J=6.80 Hz, 2H), 3.09-3.07 (m, 1H), 2.75 (t, J=6.80 Hz, 2H), 2.28-2.27 (m, 3H), 2.03-1.92 (m, 3H), 0.20 (s, 9H).

### Step 2

Into a 25 mL two-neck round-bottom flask containing a well-stirred solution of 1-[5-fluoro-1-methyl-6-(4-trimethylsilyloxycyclohex-3-en-1-yl)indazol-3-yl]hexahydropyrimidine-2,4-dione (2, 1.1 g, 2.55 mmol) in acetonitrile (10 mL) was added Selectfluor (1.36 g, 3.83 mmol) at 0° C. under nitrogen atmosphere and stirred for 1 h at the same temperature. After completion of the reaction, the reaction mixture was diluted with water (30 mL) and extracted with ethyl acetate (2×50 mL). The combined organic layer was dried over anhydrous Na₂SO₄, filtered, and concentrated under reduced pressure to afford a crude. The crude compound was purified by column chromatography (25 g, 230-400 silica-gel) using 0-100% ethyl acetate/hexane as a mobile phase while compound was eluted at 50-80% of mobile phase to afford 1-[5-fluoro-6-(3-fluoro-4-oxo-cyclohexyl)-1-methyl-indazol-3-yl]hexahydropyrimidine-2,4-dione (B-3, 0.500 g, 1.33 mmol, 52% yield) as an off-white semi-solid. ¹H NMR (400 MHz, DMSO-d₆): δ 10.56 (s, 1H), 7.71 (d, J=6.00 Hz, 1H), 7.42 (d, J=11.20 Hz, 1H), 4.04-3.99 (m, 4H), 3.91 (t, J=6.80 Hz, 2H), 3.69-3.65 (m, 1H), 2.95-2.85 (m, 1H), 2.75 (t, J=6.80 Hz, 2H), 2.47-2.31 (m, 3H), 2.30-2.10 (m, 2H).

### Synthesis B4: Synthesis of 1-[6-[(4S)-3,3-difluoro-4-piperidyl]-5-fluoro-1-methyl-indazol-3-yl]hexahydropyrimidine-2,4-dione (B-4)

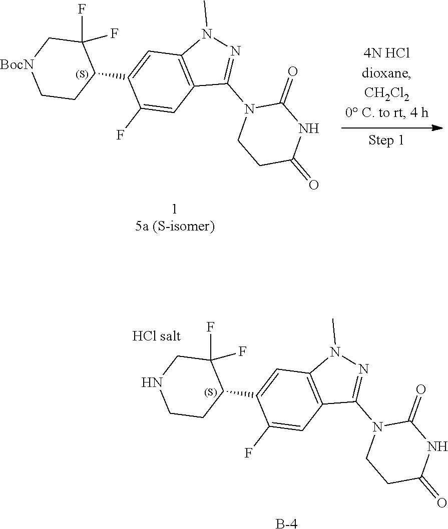

### Step 1

Into a 50 mL single-neck round-bottom flask containing a well-stirred solution of tert-butyl (4R)-4-[3-(2,4-dioxohexahydropyrimidin-1-yl)-5-fluoro-1-methyl-indazol-6-yl]-3,3-difluoro-piperidine-1-carboxylate (1, 0.26 g, 540.02 mol) in anhydrous dichloromethane (5 mL) was added Hydrogen chloride solution 4.0M in 1,4-dioxane (59.13 mg, 1.62 mmol) at 0° C. under nitrogen atmosphere. The resulting suspension was stirred at room temperature for 4 hours. After completion of the reaction, the reaction mixture was concentrated under reduced pressure to get a crude residue which was triturated with methyl tert-butyl ether (2×4 mL) to afford 1-[6-[(4S)-3,3-difluoro-4-piperidyl]-5-fluoro-1-methyl-indazol-3-yl]hexahydropyrimidine-2,4-dione (B-4, 225 mg, 537.55 mol, 99% yield, HCl salt) as an off-white solid. UPLC-MS (ES+): m/z 382.2 [M+H]⁺.

### Synthesis B5: Synthesis of 1-[6-[(4R)-3,3-difluoro-4-piperidyl]-5-fluoro-1-methyl-indazol-3-yl]hexahydropyrimidine-2,4-dione (B-5)

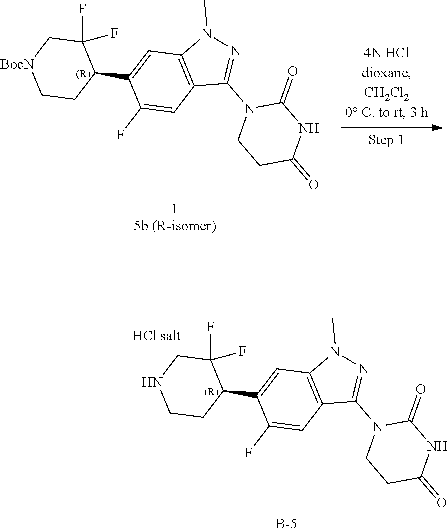

### Step 1

Into a 50 mL single-neck round-bottom flask containing a well-stirred solution of tert-butyl (4R)-4-[3-(2,4-dioxohexahydropyrimidin-1-yl)-5-fluoro-1-methyl-indazol-6-yl]-3,3-difluoro-piperidine-1-carboxylate (1, 750 mg, 1.56 mmol) in anhydrous dichloromethane (3 mL) was added 4.0 M hydrogen chloride solution in 1,4-dioxane (4 M, 3.89 mL) at 0° C. under nitrogen atmosphere. The resulting suspension was stirred at room temperature for 3 hours. After completion of the reaction, the reaction mixture was concentrated under reduced pressure to get a crude residue which was triturated with methyl tert-butyl ether (2×4 mL) to afford 1-[6-[(4R)-3,3-difluoro-4-piperidyl]-5-fluoro-1-methyl-indazol-3-yl]hexahydropyrimidine-2,4-dione (B-5, 650 mg, 1.55 mmol, 99% yield, HCl salt) as an off-white solid. UPLC-MS (ES+): m/z 382.2 [M+H]⁺.

### Synthesis B6: Synthesis of 1-[6-(3,3-difluoro-4-piperidyl)-5-fluoro-1-methyl-indazol-3-yl]hexahydropyrimidine-2,4-dione (B-6)

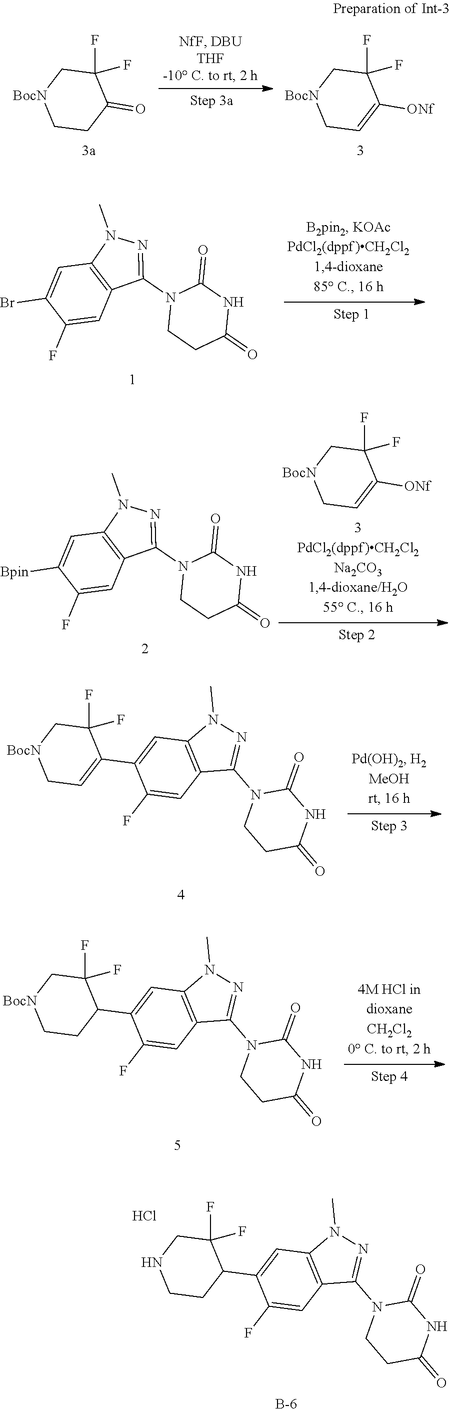

### Synthesis of int-3

### Step 3a

Into a 25 mL two-neck round-bottom flask containing a well-stirred solution of tert-butyl 3,3-difluoro-4-oxo-piperidine-1-carboxylate (3a, 100 mg, 425.12 mol) in THF (2 mL) was added 1,8-diazabicyclo(5.4. 0)undec-7-ene (1.28 mmol, 190.35 L) at −10° C. and stirred for 10 min at the same temperature. Subsequently, perfluorobutanesulfonyl fluoride (1.28 mmol, 220.16 L) was added to the reaction mixture at −10° C. and resulting reaction mixture was stirred at room temperature for 2 hours. After completion of the reaction, the reaction mixture was diluted with ethyl acetate (5 mL) and washed with water (2×5 mL). Combined organic layer was dried over anhydrous Na₂SO₄, filtered and concentrated under reduced pressure to afford a crude residue which was purified by flash silica-gel (230-400 mesh) column using 0-100% ethyl acetate/petroleum ether as an eluent to afford tert-butyl 3,3-difluoro-4-(1,1,2,2,3,3,4,4,4-nonafluorobutylsulfonyloxy)-2,6-dihydropyridine-1-carboxylate (3, 70 mg, 135.31 mol, 32% yield) as a colorless liquid. ¹H NMR (400 MHz, DMSO-d₆): δ 6.85 (s, 1H), 4.25 (d, J=3.60 Hz, 2H), 4.06 (t, J=11.20 Hz, 2H), 1.43 (s, 9H).

### Step 1

Into a 100 mL single-neck round-bottom flask containing a well-stirred solution of 1-(6-bromo-5-fluoro-1-methyl-indazol-3-yl)hexahydropyrimidine-2,4-dione (1, 1 g, 2.93 mmol) dissolved in 1,4-dioxane (20 mL) was added bis(pinacolato)diboron (1.68 g, 4.69 mmol), KOAc (863.08 mg, 8.79 mmol) at room temperature under nitrogen atmosphere and reaction mixture was degassed by bubbling nitrogen gas for 10 min. Subsequently, Pd(dppf)Cl₂.DCM (239.39 mg, 293.14 mol) was added and resulting reaction mixture was stirred at 85° C. for 16 hours. After completion of the reaction, the reaction mixture was diluted with ethyl acetate (30 mL), filtered through a celite pad, and pad was washed with ethyl acetate (20 mL). Filtrate was concentrated under reduced pressure to obtain a crude residue which was triturated with petroleum ether (2×30 mL) and 10% ethyl acetate/petroleum ether (2×25 mL) and dried under vacuum to afford 1-[5-fluoro-1-methyl-6-(4,4,5,5-tetramethyl-1,3,2-dioxaborolan-2-yl)indazol-3-yl]hexahydropyrimidine-2,4-dione (2, 1.14 g, 2.94 mmol) as a pale brown solid. ¹H NMR (400 MHz, DMSO-d₆): δ 10.57 (s, 1H), 7.85 (d, J=4.40 Hz, 1H), 7.39-7.36 (m, 1H), 4.05 (s, 3H), 3.92 (t, J=6.8 Hz, 2H), 2.77 (t, J=6.8 Hz, 2H), 1.34 (s, 12H).

### Step 2

Into a 50 mL single-neck round-bottom flask containing a well-stirred solution of 1-[5-fluoro-1-methyl-6-(4,4,5,5-tetramethyl-1,3,2-dioxaborolan-2-yl)indazol-3-yl]hexahydropyrimidine-2,4-dione (2, 1.14 g, 2.94 mmol) dissolved in a mixture of 1,4-dioxane (15 mL) and water (4 mL) was added tert-butyl 3,3-difluoro-4-(((perfluorobutyl)sulfonyl)oxy)-3,6-dihydropyridine-1(2H)-carboxylate (3, 1.52 g, 2.94 mmol), Na₂CO₃ (778.13 mg, 7.34 mmol) at room temperature under nitrogen atmosphere and the resulting mixture was degassed by bubbling nitrogen gas 10 min. Subsequently, [1,1′-Bis(diphenylphosphino)ferrocene]dichloropalladium(II) dichloromethane (239.82 mg, 293.66 mol) was added and the resulting reaction mixture was heated at 55° C. for 16 hours. After completion of the reaction, the reaction mixture was concentrated under reduced pressure to get a crude residue, obtained crude compound was dissolved in ethyl acetate (50 mL) and washed with water (20 mL). Organic layer was separated and dried over anhydrous Na₂SO₄, filtered, and concentrated under reduced pressure to get a crude residue which was purified by flash silica-gel (230-400 mesh) column using 0-100% ethyl acetate/petroleum ether as an eluent to afford tert-butyl 4-[3-(2,4-dioxohexahydropyrimidin-1-yl)-5-fluoro-1-methyl-indazol-6-yl]-3,3-difluoro-2,6-dihydropyridine-1-carboxylate (4, 950 mg, 1.76 mmol, 60% yield) as a pale brown solid. UPLC-MS (ES+): m/z 680.2 [M+H]⁺.

### Step 3

Into a 500 mL single-neck round-bottom flask containing a well-stirred solution of tert-butyl 4-[3-(2,4-dioxohexahydropyrimidin-1-yl)-5-fluoro-1-methyl-indazol-6-yl]-3,3-difluoro-2,6-dihydropyridine-1-carboxylate (4, 950 mg, 1.98 mmol) dissolved in methanol (20 mL) was added 10% Pd(OH)₂ on carbon (600 mg, 1.98 mmol, 20% by weight and 50% water) at room temperature under nitrogen atmosphere. The resulting suspension was stirred at room temperature under a hydrogen (bladder) atmosphere for 16 hours. After completion of the reaction, the reaction mixture was filtered through a celite pad, and the pad was washed with methanol (50 mL). Filtrate was concentrated under reduced pressure to afford tert-butyl 4-[3-(2,4-dioxohexahydropyrimidin-1-yl)-5-fluoro-1-methyl-indazol-6-yl]-3,3-difluoro-piperidine-1-carboxylate (5, 900 mg, 1.83 mmol, 92% yield) as an off-white solid. UPLC-MS(ES+): m/z 426 [M−^{t}Bu+H]⁺.

### Step 4

Into a 25 mL single-neck round-bottom flask containing a well-stirred solution of tert-butyl 4-[3-(2,4-dioxohexahydropyrimidin-1-yl)-5-fluoro-1-methyl-indazol-6-yl]-3,3-difluoro-piperidine-1-carboxylate (5, 0.9 g, 1.87 mmol) dissolved in dichloromethane (10 mL) was added 4M HCl in 1,4-dioxane (5.61 mmol, 10 mL) at 0° C. slowly under nitrogen atmosphere. The resulting reaction mixture was stirred at room temperature for 2 hours. After completion of the reaction, solvents were removed under reduced pressure to get crude product which was triturated with methyl tert-butyl ether (20 mL) and dried under reduced pressure to obtain 1-[6-(3,3-difluoro-4-piperidyl)-5-fluoro-1-methyl-indazol-3-yl]hexahydropyrimidine-2,4-dione (B-6, 780 mg, 1.79 mmol, 96% yield, HCl satl) as an off-white solid. UPLC-MS(ES+): m/z 382.4 [M+H]⁺.

### Separation of tert-butyl (4R)-4-[3-(2,4-dioxohexahydropyrimidin-1-yl)-5-fluoro-1-methyl-indazol-6-yl]-3,3-difluoro-piperidine-1-carboxylate and tert-butyl (4S)-4-[3-(2,4-dioxohexahydropyrimidin-1-yl)-5-fluoro-1-methyl-indazol-6-yl]-3,3-difluoro-piperidine-1-carboxylate

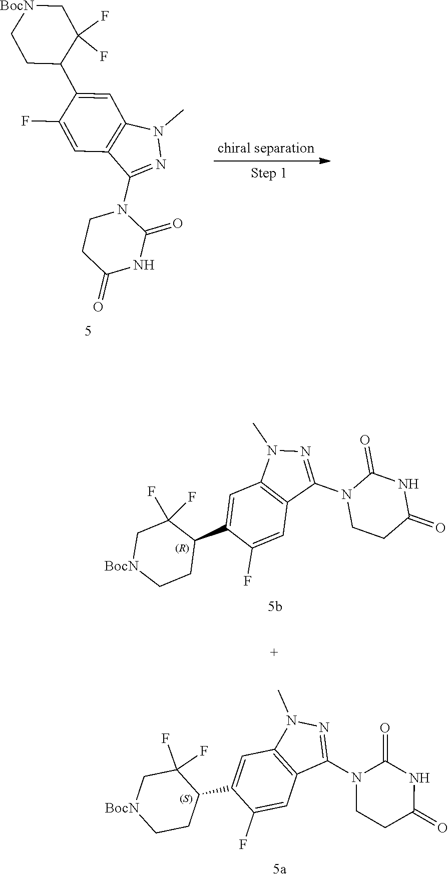

### Step-1: Chiral SFC separation

The isomeric mixture of tert-butyl 4-[3-(2,4-dioxohexahydropyrimidin-1-yl)-5-fluoro-1-methyl-indazol-6-yl]-3,3-difluoro-piperidine-1-carboxylate (5, 2.20 g, 4.57 mmol) was purified by chiral SFC following the below method.

### Method of Chiral SFC Analysis

### SFC method for chiral separation

Instrument: PIC 22-016
Column: LUX A1-(250*30)mm, 5 μm
Mobile Phase: CO₂: IPA[60:40]
Total Flow: 100 ml/min
Back pressure: 100 bar
Wavelength: 220 nm
Cycle time: 7.5 min

Fast eluting isomer (Peak-1): Fast eluting compound (Peak-1, t_{R}=3.11 min) was collected and concentrated under reduced pressure to afford tert-butyl (4S)-4-[3-(2,4-dioxohexahydropyrimidin-1-yl)-5-fluoro-1-methyl-indazol-6-yl]-3,3-difluoro-piperidine-1-carboxylate (650 mg, 1.35 mmol, 30% yield) as an off-white solid. Peak-1 was designated as an S-isomer based on preliminary data. LCMS (ES+): m/z 482.20 [M+H]⁺

Late eluting isomer (Peak-2): Late eluting compound (Peak-2, t_{R}=4.45 min) was collected and concentrated under reduced pressure to afford tert-butyl (4R)-4-[3-(2,4-dioxohexahydropyrimidin-1-yl)-5-fluoro-1-methyl-indazol-6-yl]-3,3-difluoro-piperidine-1-carboxylate (700 mg, 1.45 mmol, 32% yield) as an off-white solid. Peak-1 was designated as an R-isomer based on preliminary data. LCMS (ES+): m/z 426.20 [M−56+H]⁺ 426.20

### Synthesis B7: Synthesis of 1-[5-fluoro-1-methyl-6-(4-oxo-1-piperidyl)indazol-3-yl]hexahydropyrimidine-2,4-dione (B-7)

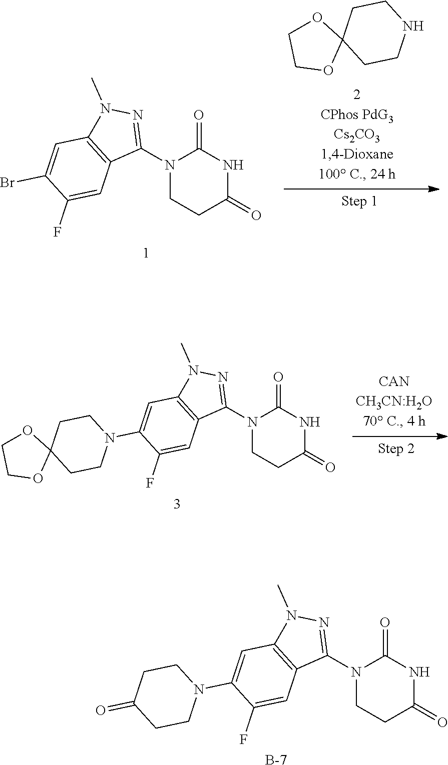

### Step 1

Into a 40 mL pressure vial containing a well-stirred solution of 1-(6-bromo-5-fluoro-1-methyl-indazol-3-yl)hexahydropyrimidine-2,4-dione (1, 800 mg, 2.35 mmol) and 1,4-dioxa-8-azaspiro[4.5]decane (2, 201.47 mg, 2.82 mmol) in anhydrous 1,4-dioxane (20 mL) was added Cs₂CO₃ (2.29 g, 7.05 mmol) at ambient temperature under nitrogen atmosphere and resulting mixture was degassed by bubbling nitrogen gas for 10 min. Subsequently, CPhos Pd G3 (189 mg, 234.52 mol) was added and resulting reaction mixture was stirred at 100° C. for 24 hours. After completion of the reaction, the reaction mixture was allowed to cool to room temperature and filtered through a pad of Celite, washed with dichloromethane (50 mL) and filtrate was concentrated under reduced pressure to get a crude residue which was purified by flash silica-gel (230-400 mesh) column using 0-10% methanol/dichloromethane as an eluent to afford 1-[6-(1,4-dioxa-8-azaspiro[4.5]decan-8-yl)-5-fluoro-1-methyl-indazol-3-yl]hexahydropyrimidine-2,4-dione (3, 200 mg, 421.4 mol, 21% yield) as an off-white solid. UPLC-MS (ES+): m/z 404.2 [M+H]⁺.

### Step 2

Into a 25 mL single-neck round-bottom flask containing a well-stirred suspension of 1-[6-(1,4-dioxa-8-azaspiro[4.5]decan-8-yl)-5-fluoro-1-methyl-indazol-3-yl]hexahydropyrimidine-2,4-dione (3, 200 mg, 495.78 mol) in acetonitrile (3 mL) and water (3 mL) was added cerium(IV) ammonium nitrate (116.06 mg, 247.89 mol) at ambient temperature under nitrogen atmosphere. Reaction mixture was stirred at 70° C. for 4 hours. After completion of the reaction, water (20 mL) was added, and mixture was extracted with ethyl acetate (2×20 mL). Combined organic layers were washed with saturated NaCl solution (20 mL), dried over anhydrous Na₂SO₄, filtered and concentrated under reduced pressure to afford 1-[5-fluoro-1-methyl-6-(4-oxo-1-piperidyl)indazol-3-yl]hexahydropyrimidine-2,4-dione (B-7, 120 mg, 303.88 mol, 61% yield) as brown sloid. UPLC-MS (ES+): m/z 360.0 [M+H]⁺.

### Synthesis B8: Synthesis of 1-[3-(2,4-dioxohexahydropyrimidin-1-yl)-5-fluoro-1-methyl-indazol-6-yl]piperidine-4-carbaldehyde (B-8)

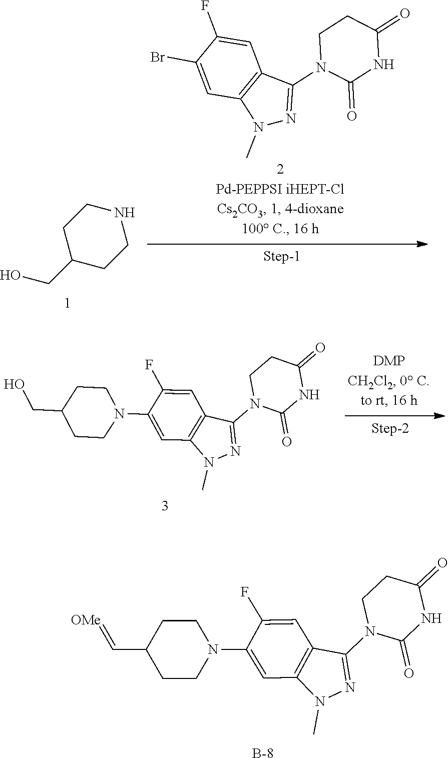

### Step 1

Into a 40 mL pressure vial containing a well-stirred solution of 4-piperidylmethanol (1, 844.04 mg, 7.33 mmol) and 1-(6-bromo-5-fluoro-1-methyl-indazol-3-yl)hexahydropyrimidine-2,4-dione (2, 1 g, 2.93 mmol) in anhydrous 1,4-dioxane (15 mL) was added Cs₂CO₃ (2.87 g, 8.79 mmol)) at ambient temperature under nitrogen atmosphere and the resulting mixture was degassed by bubbling nitrogen gas into the reaction mixture for 2 min. Subsequently, Pd-Peppsi-iheptCl (142.47 mg, 146.57 mol) was added and stirred at 100° C. for 16 hours. After completion of the reaction, the reaction mixture was quenched with water (30 mL) and extracted with ethyl acetate (2×20 mL). The organic layer was dried over Na₂SO₄, filtered and concentrated under reduced pressure to give crude which was purified by flash column chromatogrpahy (silica gel 230-400 mesh) with 0-100% methanol/dichloromethane to afford 1-[5-fluoro-6-[4-(hydroxymethyl)-1-piperidyl]-1-methyl-indazol-3-yl]hexahydropyrimidine-2,4-dione (3, 200 mg, 485.03 mol, 17% yield) as an off-white solid. UPLC-MS (ES+): m/z 376.20 [M+H]⁺.

### Step 2

Into a 50 mL single neck round bottom flask containing a well stirred solution of 1-[5-fluoro-6-[4-(hydroxymethyl)-1-piperidyl]-1-methyl-indazol-3-yl]hexahydropyrimidine-2,4-dione (3, 70 mg, 186.47 mol) in dichloromethane (4 mL) was added Dess-Martin Periodinane (158.18 mg, 372.94 mol) at 0° C. under nitrogen atmosphere. The resulting reaction mixture was allowed to room temperature and stirred for 16 hours. After completion of the reaction, the reaction mixture was diluted with dichloromethane (30 mL), washed with water (2×50 mL) and NaHCO₃ solution (10 mL). Combined organic layer was dried over anhydrous Na₂SO₄ and concentrated under reduced pressure to get the crude which was washed with hexane (20 mL) and dried under reduced pressure to afford 1-[3-(2,4-dioxohexahydropyrimidin-1-yl)-5-fluoro-1-methyl-indazol-6-yl]piperidine-4-carbaldehyde (B-8, 95 mg, 42.11 mol, 23% yield) as a colorless liquid. UPLC-MS (ES+): m/z 374.20 [M+H]⁺.

### Synthesis B9: Synthesis of 1-[5-fluoro-1-methyl-6-(2-oxo-7-azaspiro[3.5]nonan-7-yl)indazol-3-yl]hexahydropyrimidine-2,4-dione (B-9)

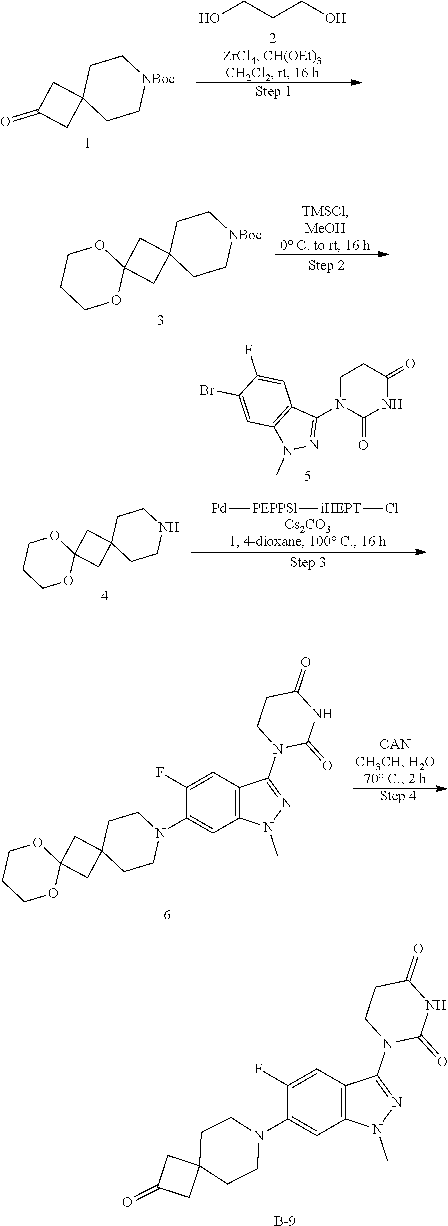

### Step 1

Into a 50 mL single-neck round-bottom flask containing a well-stirred solution of tert-butyl 2-oxo-7-azaspiro[3.5]nonane-7-carboxylate (1, 10 g, 41.79 mmol) in dichloromethane (60 mL) were added propane-1,3-diol (2, 83.57 mmol, 6.04 mL), triethyl orthoformate (45.97 mmol, 7.65 mL) and ZrCl₄ (4.87 g, 20.89 mmol) at room temperature under nitrogen atmosphere and stirred for 16 h at the same temperature. After completion of the reaction, the reaction mixture was diluted with water (50 mL) and extracted with dichloromethane (300 mL). Organic layer was washed with water (50 mL), dried over anhydrous Na₂SO₄, filtered and concentrated under reduced pressure to afford a crude residue which was purified by flash (silica-gel 230-400 mesh; 340 g) column with 0-100% ethyl acetate/hexane to afford tert-butyl 9,13-dioxa-3-azadispiro[5.1.5⁸.1⁶]tetradecane-3-carboxylate (3, 5 g, 16.14 mmol, 39% yield) as a colorless liquid. UPLC-MS (ES+): m/z 198.4 [M+H-Boc]⁺.

### Step 2

Into a 50 mL two-neck round-bottom flask containing a well-stirred solution of tert-butyl 9,13-dioxa-3-azadispiro[5.1.5⁸1⁶]tetradecane-3-carboxylate (3, 5 g, 16.14 mmol) in methanol (100 mL) was added TMS-C₁ (80.70 mmol, 10.24 mL) at 0° C. under nitrogen atmosphere. Resulting reaction mixture was stirred at room temperature for 16 hours. After completion of the reaction, the reaction mixture was concentrated under reduced pressure to afford a crude residue which was triturated with methyl tert-butyl ether (50 mL) and dried under reduced pressure to get crude product (HCl salt). Crude was dissolved in 20% methanol in dichloromethane (300 mL) and basified (pH was adjusted to 8) by adding (Amberlyst™ A-21, ion exchange) and stirring for 1 hour. Reaction mixture was filtered through sintered funnel, filtrate was concentrated under reduced pressure to afford crude 9,13-dioxa-3-azadispiro[5.1.5⁸0.1⁶]tetradecane (4, 3.5 g, 15.97 mmol, 99% yield) as an off-white solid. UPLC-MS (ES+): m/z 198.4 [M+H]⁺.

### Step 3

Into a 40 mL pressure vial containing a well-stirred solution of 9,13-dioxa-3-azadispiro[5.1.5₈0.1⁶]tetradecane (4, 578.28 mg, 2.93 mmol), 1-(6-bromo-5-fluoro-1-methyl-indazol-3-yl)hexahydropyrimidine-2,4-dione (5, 0.5 g, 1.47 mmol) in 1,4-dioxane (10 mL) was added Cs₂CO₃ (1.43 g, 4.40 mmol) at room temperature under nitrogen atmosphere. Reaction mixture was degassed by bubbling N2 gas for 5 minutes. Finally, Pd-PEPPSI-iHept-Cl (142.58 mg, 146.57 mol) was added and stirred at 100° C. for 16 hours. After completion of the reaction, the reaction mixture was filtered through a Celite pad and washed with ethyl acetate (30 mL), filtrate was concentrated under reduced pressure to afford a crude residue which was purified by flash (silica-gel 230-400 mesh; 25 g) column with 0-100% ethyl acetate/hexane to afford 1-[6-(9,13-dioxa-3-azadispiro[5.1.5⁸.1⁶]tetradecan-3-yl)-5-fluoro-1-methyl-indazol-3-yl]hexahydropyrimidine-2,4-dione (6, 250 mg, 393.45 mol, 27% yield) as an off-white solid. UPLC-MS (ES+): m/z 458.1 [M+H]⁺.

### Step 4

Into a 40 mL pressure vial containing a well-stirred solution of 1-[6-(9,13-dioxa-3-azadispiro[5.1.5⁸.1⁶]tetradecan-3-yl)-5-fluoro-1-methyl-indazol-3-yl]hexahydropyrimidine-2,4-dione (6, 1 g, 1.42 mmol) in acetonitrile (9 mL) and water (4 mL) was added cerium (IV) ammonium nitrate, 98+% (332.59 mg, 710.39 mol) at room temperature. Resulting reaction mixture was stirred at 70° C. for 2 hours. After completion of the reaction, the reaction mixture was cooled to room temperature and extracted with ethyl acetate (2×50 mL). Combined organic layer was washed with water (2×20 mL), dried over anhydrous Na₂SO₄, filtered and concentrated under reduced pressure to afford a crude 1-[5-fluoro-1-methyl-6-(2-oxo-7-azaspiro[3.5]nonan-7-yl)indazol-3-yl]hexahydropyrimidine-2,4-dione (B-9, 659 mg, 1.42 mmol, 100% yield) as an off-white solid. UPLC-MS (ES+): m/z 400.1 [M+H]⁺.

### Synthesis B10: Synthesis of 1-[6-(2,7-diazaspiro[3.5]nonan-7-yl)-5-fluoro-1-methyl-indazol-3-yl]hexahydropyrimidine-2,4-dione (B-10)

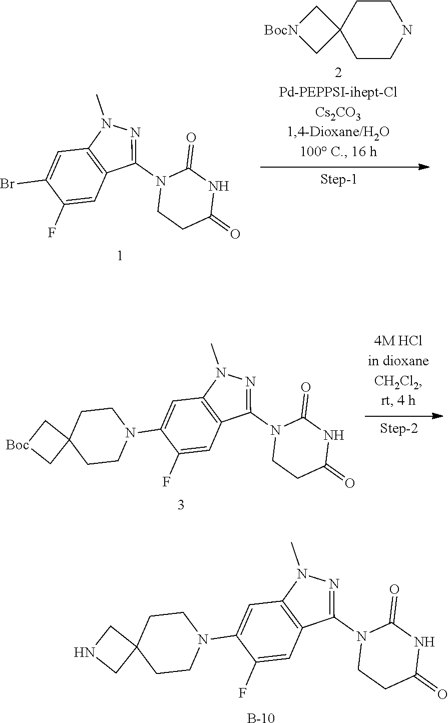

### Step 1

Into a 50 mL sealed tube containing a well-stirred solution of 1-(6-bromo-5-fluoro-1-methyl-indazol-3-yl)hexahydropyrimidine-2,4-dione (1, 1 g, 2.93 mmol) and tert-butyl 2,7-diazaspiro[3.5]nonane-2-carboxylate (2, 663.42 mg, 2.93 mmol) in anhydrous 1,4-dioxane (10 mL) and water (3 mL) was added Cs₂CO₃ (1.43 g, 4.40 mmol) at room temperature under nitrogen atmosphere. The reaction mixture was degassed with nitrogen gas for 10 min. Subsequently, Pd-Peppsi-iheptCl (284.93 mg, 293.14 mol) was added to the mixture and the resulting mixture was stirred at 100° C. for 16 hours. After completion of the reaction, the reaction mixture was cooled to room temperature and poured into water (50 mL) and extracted with ethyl acetate (2×100 mL). The combined organic layer was washed with saturated NaCl (10 mL), dried over anhydrous Na₂SO₄, filtered and concentrated under reduced pressure to get a crude residue which was purified by flash column chromatography (50 g, 230-400 silica-gel mesh) with 0-10% methanol in dichloromethane to afford tert-butyl 7-[3-(2,4-dioxohexahydropyrimidin-1-yl)-5-fluoro-1-methyl-indazol-6-yl]-2,7-diazaspiro[3.5]nonane-2-carboxylate (3, 900 mg, 1.51 mmol, 52% yield) as an off-white solid. UPLC-MS (ES+): m/z 485.2 [M+H]⁺.

### Step 2

Into a 100 mL single-neck round bottom flask containing a well-stirred solution of tert-butyl 7-[3-(2,4-dioxohexahydropyrimidin-1-yl)-5-fluoro-1-methyl-indazol-6-yl]-2,7-diazaspiro[3.5]nonane-2-carboxylate (3, 0.9 g, 1.85 mmol) in anhydrous dichloromethane (15 mL) was added 4M HCl in 1,4-dioxane (1.85 mmol, 4.0 mL) at 0° C. under nitrogen atmosphere. The resulting mixture was stirred at room temperature for 4 hours. After completion of the reaction, the reaction mixture was concentrated under reduced pressure to get a crude residue, which was washed with methyl tert-butyl ether (2×5 mL) and dried under reduced pressure to afford 1-[6-(2,7-diazaspiro[3.5]nonan-7-yl)-5-fluoro-1-methyl-indazol-3-yl]hexahydropyrimidine-2,4-dione (B-10, 0.850 g, 1.81 mmol, 98% yield, HCl salt) as an off-white solid. UPLC-MS (ES+): m/z 387.3 [M+H]⁺.

### Synthesis B11: Synthesis of 1-[5-fluoro-1-methyl-6-(6-oxo-2-azaspiro[3.3]heptan-2-yl)indazol-3-yl]hexahydropyrimidine-2,4-dione (B-11)

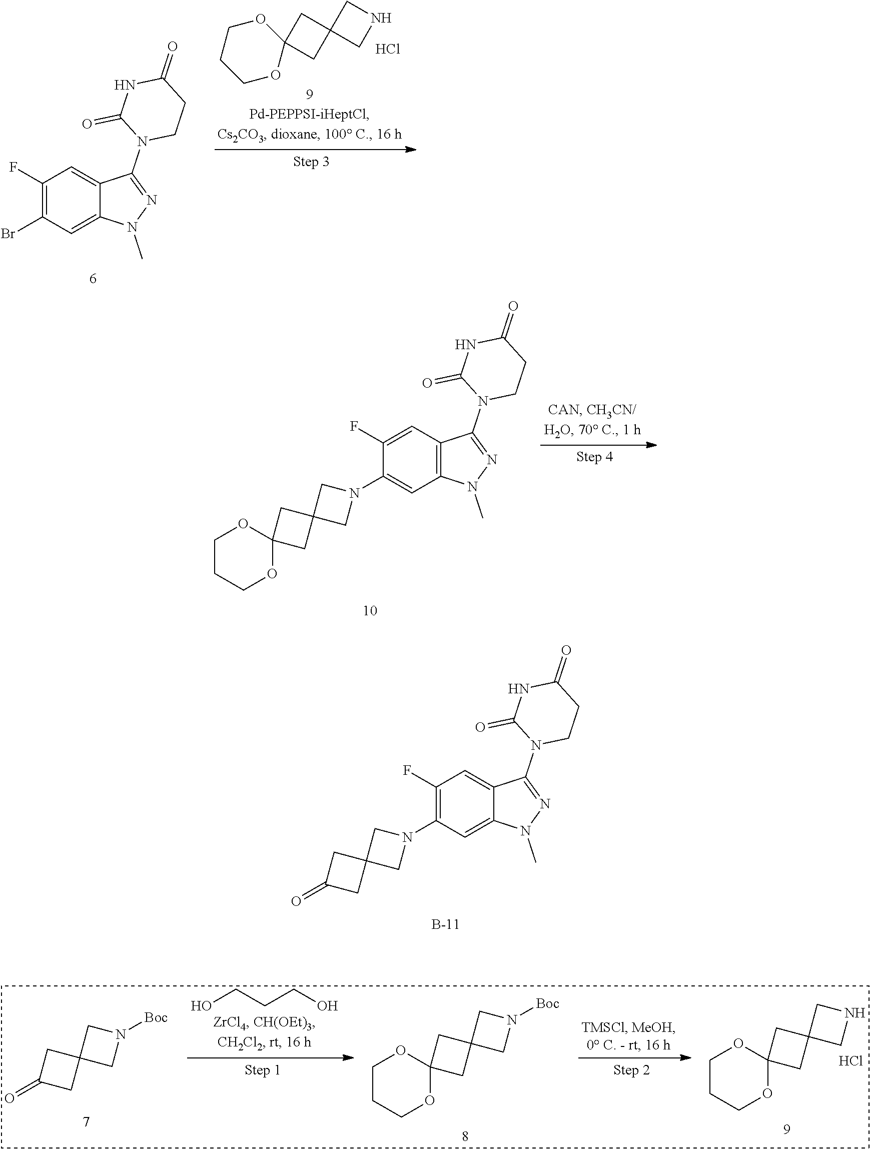

### Step 1

To a solution of tert-butyl 6-oxo-2-azaspiro[3.3]heptane-2-carboxylate (7; 100 g, 473.36 mmol) in dichloromethane (1500 mL), were added propane-1,3-diol (68.4 mL, 946.71 mmol), triethyl orthoformate (86.6 mL, 520.69 mmol) and followed by zirconium(IV) chloride (2.0 mL, 23.67 mmol). The resulting mixture was stirred at room temperature for 16 hours. The reaction mixture was concentrated under reduced pressure to give a crude residue, which was treated with water (1000 ml), the solid precipitated was filtered, washed with water (250 mL), and dried under vacuum to afford tert-butyl 7,11-dioxa-2-azadispiro[3.1.5⁶.1⁴]dodecane-2-carboxylate (8; 110 g, 407.27 mmol, 86% yield) as an off-white solid. LC-MS (ES*): m/z 170.2 [M−COO^{t}Bu+H]⁺.

### Step 2

To a solution of tert-butyl 7,11-dioxa-2-azadispiro[3.1.56.14]dodecane-2-carboxylate (8; 50 g, 185.12 mmol) in methanol (400 mL), cooled to 0° C., was added chlorotrimethylsilane (117.5 mL, 925.61 mmol). The resulting mixture was stirred at room temperature for 16 hours. The volatiles were evaporated under reduced pressure to afford 7,11-dioxa-2-azadispiro[3.1.56.14]dodecane hydrochloride (9; 38 g, 183.20 mmol, 99% yield) as an off-white solid. LC-MS (ES+): m/z 170.2 [M+H]⁺.

### Step 3

To a suspension of 1-(6-bromo-5-fluoro-1-methyl-indazol-3-yl)hexahydropyrimidine-2,4-dione (6; 5 g, 14.41 mmol) and 7,11-dioxa-2-azadispiro[3.1.5⁶.1⁴]dodecane hydrochloride (9; 3.89 g, 18.73 mmol) in 1,4-dioxane (125 mL), was added Cs₂CO₃ (14.09 g, 43.23 mmol). The resulting mixture was degassed with nitrogen for 10 min before Pd-PEPPSI-iHeptCl (420.99 mg, 432.32 mol) was added to the mixture. The resulting mixture was heated at 100° C. for 16 hours. The reaction mixture was brought to room temperature and diluted with ethyl acetate (100 mL) and filtered through a pad of celite. The celite was washed with THF/1,4-dioxane/ethyl acetate (1:1:1 ratio, 500 mL), and the filtrate was concentrated under reduced pressure to obtain crude 1-[6-(7,11-dioxa-2-azadispiro[3.1.56.14]dodecan-2-yl)-5-fluoro-1-methyl-indazol-3-yl]hexahydropyrimidine-2,4-dione (10; 6 g, 9.42 mmol, 65% yield) as a yellow gum.

4×5 g batches were prepared and combined together for purification. The crude product was purified by flash chromatography (Silica gel, 230-400 mesh) and eluted with 0-10% methanol in ethyl acetate, while the desired product was eluted with 5-8% methanol in ethyl acetate. The fractions containing the product were pooled and concentrated under reduced pressure to afford 1-[6-(7,11-dioxa-2-azadispiro[3.1.56.14]dodecan-2-yl)-5-fluoro-1-methyl-indazol-3-yl]hexahydropyrimidine-2,4-dione (10; 10 g, 22.85 mmol, 52% yield) as an off-white solid. LC-MS (ES+): m/z 430.2 [M+H]⁺.

### Step 4

To a suspension of 1-[6-(7,11-dioxa-2-azadispiro[3.1.5₆.1⁴]dodecan-2-yl)-5-fluoro-1-methyl-indazol-3-yl]hexahydropyrimidine-2,4-dione (10; 10 g, 22.85 mmol) in acetonitrile (150 mL) and water (150 mL), was added ceric ammonium nitrate (6.26 g, 11.42 mmol) at room temperature. The resulting solution was stirred at 70° C. for 1 hour. The reaction mixture was brought to room temperature, diluted with ethyl acetate (150 mL), and washed with water (100 mL). The organic phase was washed with saturated NaCl solution (75 mL) and dried over anhydrous Na₂SO₄. The solution was filtered and concentrated under reduced pressure to give a crude product, which was purified by flash chromatography (Silica gel, 100-200 mesh) and eluted using 0-10% methanol in dichloromethane, while the desired compound was eluted at 2-3% methanol in dichloromethane. The fractions containing the product were pooled and concentrated under reduced pressure. The product was further triturated with acetonitrile (30 mL), filtered, and dried under vacuum to afford 1-[5-fluoro-1-methyl-6-(6-oxo-2-azaspiro[3.3]heptan-2-yl)indazol-3-yl]hexahydropyrimidine-2,4-dione (B-11; 5.8 g, 14.22 mmol, 62% yield) as an off-white solid. LC-MS (ES+): m/z 372.2 [M+H]⁺. ¹H NMR (400 MHz, DMSO-d₆): δ 10.52 (s, 1H), 7.29 (d, J=12.0 Hz, 1H), 6.57 (d, J=7.6 Hz, 1H), 4.17 (s, 4H), 3.90 (s, 3H), 3.86 (t, J=4.0 Hz, 2H), 3.38 (s, 4H), 2.73 (t, J=6.8 Hz, 2H).

### Synthesis B12: Synthesis of 3-[4-(2,6-diazaspiro[3.3]heptan-2-yl)-3-fluoro-anilino]piperidine-2,6-dione (B-12)

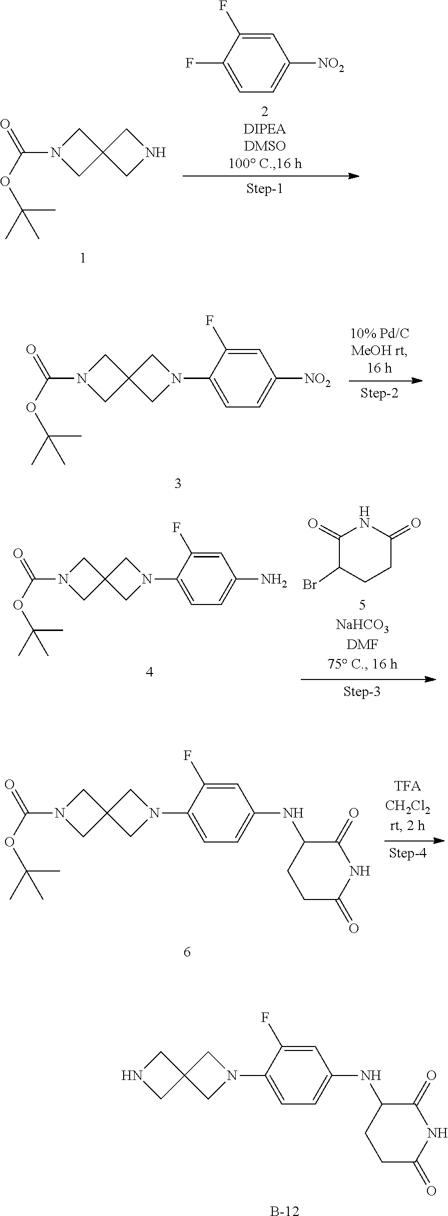

### Step 1

Into a 250 mL single-neck round bottom flask containing a mixture of tert-butyl 2,6-diazaspiro[3.3]heptane-2-carboxylate (1, 3.0 g, 10.41 mmol, Oxalic acid salt) in anhydrous DMSO (75 mL) were added N,N-diisopropylethylamine (10.41 mmol, 1.81 mL) and 1,2-difluoro-4-nitro-benzene (2, 1.66 g, 10.41 mmol, 1.15 mL) at room temperature under nitrogen atmosphere and the reaction mixture was stirred at 100° C. for 16 hours. After completion of the reaction, the reaction mixture was quenched with cold water (50 mL) and after stirring at room temperature for 30 min, a solid precipitate formed. Precipitated solid was filtered and washed with hexane (50 mL), dried under reduced pressure to afford tert-butyl 6-(2-fluoro-4-nitro-phenyl)-2,6-diazaspiro[3.3]heptane-2-carboxylate (3, 3.5 g, 10.35 mmol, 99% yield) as an off-white solid. UPLC-MS (ES+): m/z 338.2 [M+H]⁺.

### Step 2

Into a 250 mL single-neck round-bottom flask containing a well-stirred solution of tert-butyl 6-(2-fluoro-4-nitro-phenyl)-2,6-diazaspiro[3.3]heptane-2-carboxylate (3, 3.5 g, 10.38 mmol) in anhydrous methanol (80 mL), reaction mixture was purged under nitrogen for 5 min, palladium 10% on carbon (3.5 g, 10.38 mmol) was added under nitrogen atmosphere and the reaction mixture was stirred at room temperature for 16 h under hydrogen pressure. After completion of the reaction, the reaction mixture was filtered on celite bed and washed with ethyl acetate (200 mL) and the filtrate was concentrated under reduced pressure to afford tert-butyl 6-(4-amino-2-fluoro-phenyl)-2,6-diazaspiro[3.3]heptane-2-carboxylate (4, 3 g, 9.70 mmol, 94% yield) as an off-white solid. UPLC-MS (ES+): m/z 308.1 [M+H]⁺.

### Step 3

Into a 100 mL sealed tube containing a well-stirred solution of tert-butyl 6-(4-amino-2-fluoro-phenyl)-2,6-diazaspiro[3.3]heptane-2-carboxylate (4, 3.0 g, 9.76 mmol) in anhydrous N,N-dimethylformamide (75 mL) were added NaHCO₃ (2.46 g, 29.28 mmol) and 3-bromopiperidine-2,6-dione (5, 5.62 g, 29.28 mmol) at ambient temperature under nitrogen atmosphere and stirred at 75° C. for 16 hours. After completion of the reaction, the reaction mixture was allowed to cool to room temperature, quenched with cold water (50 mL) and extracted with ethyl acetate (2×50 mL). The combined organic layers were washed with cold water (2×100 mL) and dried over anhydrous Na₂SO₄, filtered and concentrated under reduced pressure to afford tert-butyl 6-[4-[(2,6-dioxo-3-piperidyl)amino]-2-fluoro-phenyl]-2,6-diazaspiro[3.3]heptane-2-carboxylate (6, 1.5 g, 2.94 mmol, 30% yield) as a pale green semi solid. UPLC-MS (ES+): m/z 419.4 [M+H]⁺.

### Step 4

Into a 50 mL single-neck round-bottom flask containing a well-stirred solution of tert-butyl 6-[4-[(2,6-dioxo-3-piperidyl)amino]-2-fluoro-phenyl]-2,6-diazaspiro[3.3]heptane-2-carboxylate (6, 1.2 g, 2.87 mmol) in dichloromethane (20 mL) was added trifluoroacetic acid (28.68 mmol, 2.19 mL) slowly at 0° C. under nitrogen atmosphere. The resulting reaction mixture was stirred at room temperature for 2 hours. After completion of the reaction, the reaction mixture was concentrated under reduced pressure to get a crude residue and the crude residue was washed with methyl tert-butyl ether (2×50 mL) to remove the non-polar impurity to afford 3-[4-(2,6-diazaspiro[3.3]heptan-2-yl)-3-fluoro-anilino]piperidine-2,6-dione (B-12, 800 mg, 1.63 mmol, 57% yield, TFA salt) as a brown gum. UPLC-MS (ES+): m/z 319.4 [M+H]⁺.

### Synthesis B13: Synthesis of 3-[3-fluoro-4-(4-oxocyclohexyl)anilino]piperidine-2,6-dione (B-13)

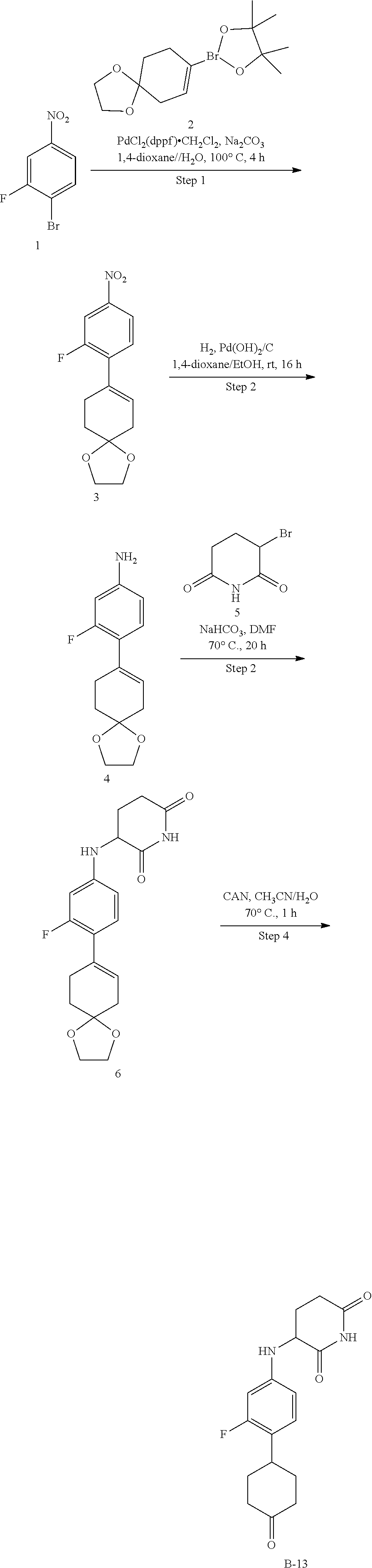

### Step 1

To a solution of 1-bromo-2-fluoro-4-nitro-benzene (1; 30 g, 136.37 mmol) and 2-(1,4-dioxaspiro[4.5]dec-7-en-8-yl)-4,4,5,5-tetramethyl-1,3,2-dioxaborolane (2; 39.92 g, 150.00 mmol) in 1,4-dioxane (250 mL) and water (50 mL), was added sodium carbonate (36.13 g, 340.92 mmol) at room temperature. The contents were degassed by bubbling nitrogen for 10 min before [1,1′-bis(diphenylphosphino)ferrocene]dichloropalladium(II), complex with dichloromethane (5.57 g, 6.82 mmol) was added to the mixture. The resulting mixture was heated at 100° C. for 4 hours. The reaction mixture was filtered through a pad of celite, and the celite was washed with ethyl acetate (2×1000 mL). The filtrate was concentrated under reduced pressure to give a crude residue, which was purified by flash chromatography (Silica gel, 230-400 mesh) eluted using 15% ethyl acetate in petroleum ether to afford 8-(2-fluoro-4-nitro-phenyl)-1,4-dioxaspiro[4.5]dec-7-ene (3; 35 g, 119.35 mmol, 88% yield) as a yellow gum. LC-MS (ES−): m/z 278.0 [M−H]⁻.

### Step 2

To a suspension of 8-(2-fluoro-4-nitro-phenyl)-1,4-dioxaspiro[4.5]dec-7-ene (3; 30 g, 102.30 mmol) in 1,4-dioxane (200 mL) and EtOH (100 mL), was added palladium hydroxide on carbon, (20 wt. % loading; 20 g, 142.41 mmol) at room temperature. The resulting suspension was stirred at room temperature under a hydrogen atmosphere (~1 kg/cm²) for 16 hours. The reaction mixture was filtered through a pad of celite and the celite was washed with ethyl acetate (2500 mL). The filtrate was concentrated under reduced pressure to afford 4-(1,4-dioxaspiro[4.5]decan-8-yl)-3-fluoro-aniline (4; 25 g, 97.09 mmol, 95% yield) as an off-white solid. LC-MS (ES*): m/z 252.2 [M+H]⁺.

### Step 3

To a solution of 4-(1,4-dioxaspiro[4.5]decan-8-yl)-3-fluoro-aniline (4; 20 g, 77.67 mmol) and 3-bromopiperidine-2,6-dione (5; 44.74 g, 233.01 mmol) in N,N-dimethylformamide (200 mL), was added sodium bicarbonate (22.84 g, 271.84 mmol) at room temperature. The resulting mixture was heated at 70° C. for 20 hours. The reaction mixture was brought to room temperature, filtered through a pad of celite, and washed with ethyl acetate (1000 mL). The filtrate was washed with water (2×300 mL), the organic phase was separated, washed with saturated NaCl solution (150 mL), and dried over anhydrous sodium sulfate, filtered, and dried to give a crude product. The crude product was triturated with methanol (150 mL) and the solid precipitated was filtered and dried under vacuum to afford 3-[4-(1,4-dioxaspiro[4.5]decan-8-yl)-3-fluoro-anilino]piperidine-2,6-dione (6; 17.0 g, 46.83 mmol, 60% yield) as an off-white solid. LC-MS (ES+): m/z 363.2 [M+H]⁺.

### Step 4

To a suspension of 3-[4-(1,4-dioxaspiro[4.5]decan-8-yl)-3-fluoro-anilino]piperidine-2,6-dione (6; 10.0 g, 27.54 mmol) in acetonitrile (50 mL) and water (50 mL), was added ceric ammonium nitrate (0.9 g, 1.93 mmol) at room temperature. The resulting mixture was heated at 70° C. for 1 hour. The reaction mixture was brought to room temperature and treated with ice-cold water (200 mL). The solid precipitated was filtered, washed with water (50 mL), and dried under vacuum to afford 3-[3-fluoro-4-(4-oxocyclohexyl)anilino]piperidine-2,6-dione (B-13; 7.8 g, 24.10 mmol, 87% yield) as a pale green solid. LC-MS (ES+): m/z 319.2 [M+H]⁺. ¹H NMR (400 MHz, DMSO-d₆): δ 10.80 (s, 1H), 7.03 (t, J=8.4 Hz, 1H), 6.48-6.45 (m, 2H), 6.04 (d, J=7.6 Hz, 1H), 4.35-4.29 (m, 1H), 3.21-3.15 (m, 1H), 2.77-2.70 (m, 1H), 2.63-2.55 (m, 3H), 2.27-2.23 (m, 2H), 2.11-2.06 (m, 1H), 2.00-1.98 (m, 2H), 1.91-1.80 (m, 3H).

### Synthesis B14: Synthesis of 3-[3-fluoro-4-(6-oxo-2-azaspiro[3.3]heptan-2-yl)anilino]piperidine-2,6-dione (B-14)

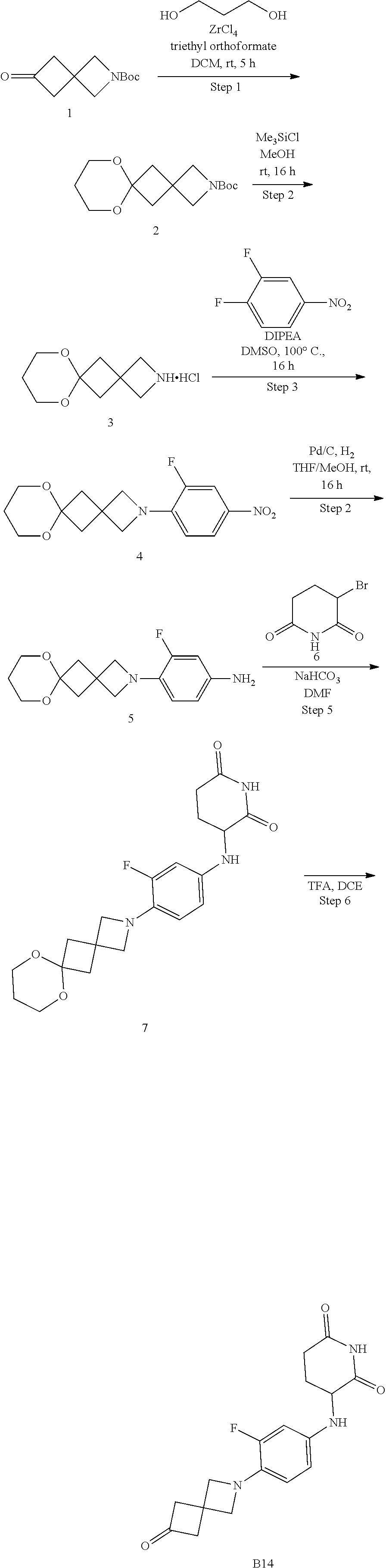

### Step 1

Into a 250 mL two-neck round-bottom flask containing a well-stirred solution of tert-butyl 6-oxo-2-azaspiro[3.3]heptane-2-carboxylate (1, 5 g, 23.67 mmol) in dichloromethane (49.90 mL) were added propane-1,3-diol (3.60 g, 47.34 mmol, 3.42 mL), triethyl orthoformate (3.86 g, 26.03 mmol, 4.33 mL) and zirconium (IV) chloride (275.77 mg, 1.18 mmol) at room temperature under nitrogen atmosphere. The reaction mixture was stirred at room temperature for 5 hours. After completion of the reaction, the reaction mixture was quenched with water (100 mL) and extracted with dichloromethane (2×100 mL). The organic phase was washed with water (3×30 mL), dried over anhydrous Na₂SO₄, filtered and concentrated under reduced pressure to afford tert-butyl 7,11-dioxa-2-azadispiro[3.1.5⁶.1⁴]dodecane-2-carboxylate (2, 5.5 g, 20.34 mmol, 86% yield) as a colorless oil. UPLC-MS (ES+): m/z 170.1 [M−Boc+H]⁺.

### Step 2

Into a 250 mL single-neck round-bottom flask containing a well-stirred solution of tert-butyl 7,11-dioxa-2-azadispiro[3.1.5⁶1⁴]dodecane-2-carboxylate (2, 5.5 g, 20.42 mmol) in methanol (55 mL) was added slowly chlorotrimethylsilane (102.10 mmol, 12.96 mL) at 0° C. under nitrogen atmosphere. The reaction mixture was stirred at room temperature for 16 hours. After completion of the reaction, the reaction mixture was concentrated under reduced pressure and triturated with methyl tert-butyl ether to remove the non-polar impurity to afford 7,11-dioxa-2-azadispiro[3.1.5⁶.1⁴]dodecane (3, 3.65 g, 17.04 mmol, 83% yield, HCl salt) as a pale yellow solid. UPLC-MS (ES+): m/z 170.3 [M+H]⁺.

### Step 3

Into 100 mL two-neck round-bottom flask containing a well-stirred solution of 1,2-difluoro-4-nitrobenzene (3, 3.4 g, 21.37 mmol, 2.37 mL) in DMSO (34 mL) were added 7,11-dioxa-2-azadispiro[3.1.56.14]dodecane (3.62 g, 21.37 mmol) and N,N-diisopropylethylamine (427.43 mmol, 74.45 mL) at room temperature under nitrogen atmosphere. The reaction mixture was stirred at 100° C. for 16 hours. After completion of the reaction, the reaction mixture was diluted with ice-cold water (50 mL) and the resulting precipitate was filtered. The filtrate was concentrated under reduced pressure to afford 2-(2-fluoro-4-nitro-phenyl)-7,11-dioxa-2-azadispiro[3.1.5⁶.1⁴]dodecane (4, 2.5 g, 7.70 mmol, 36% yield) as a yellow solid. LC-MS(ES+): m/z 309.2 [M+H]⁺.

### Step 4

Into a 250 mL single-neck round-bottom flask containing a well-stirred solution of 2-(2-fluoro-4-nitro-phenyl)-7,11-dioxa-2-azadispiro[3.1.5⁶.1⁴]dodecane (4, 2.5 g, 8.11 mmol) in THF (50 mL) and methanol (50 mL) was degassed with nitrogen for 5 min. palladium on carbon 50% wet (1.37 g) was added to the reaction mixture at room temperature and stirred under H₂ bladder at room temperature for 16 hours. After completion of the reaction, the reaction mixture was filtered through a Celite bed and the filtrate was concentrated under reduced pressure to afford 4-(7,11-dioxa-2-azadispiro[3.1.5⁶.1⁴]dodecan-2-yl)-3-fluoro-aniline (5, 2 g, 5.61 mmol, 69% yield) as an off-white solid. UPLC-MS (ES+): m/z 279.2 [M+H]⁺.

### Step 5

Into a 250 mL sealed tube containing a well-stirred solution of 4-(7,11-dioxa-2-azadispiro[3.1.5⁶.1⁴]dodecan-2-yl)-3-fluoro-aniline (5, 2 g, 7.19 mmol) in N,N-dimethylformamide (25 mL) were added 3-bromopiperidine-2,6-dione (6, 5.52 g, 28.74 mmol) and NaHCO₃ (4.83 g, 57.49 mmol) at room temperature under nitrogen atmosphere. The reaction mixture was stirred at 70° C. for 16 hours. After completion of the reaction, the reaction mixture was diluted with ice cold water (50 mL), extracted with ethyl acetate (3×50 mL). Combined organic layers were washed with ice cold water (3×10 mL), dried over Na₂SO₄, filtered and concentrated under reduced pressure to obtain a crude residue. Crude material was purified by flash silica gel (230-400 mesh) column chromatography by eluting with 0-70% ethyl acetate in pet-ether to afford 3-[4-(7,11-dioxa-2-azadispiro[3.1.56.1]dodecan-2-yl)-3-fluoro-anilino]piperidine-2,6-dione (7, 1.7 g, 4.23 mmol, 59% yield) as a pale green semi-solid. UPLC-MS (ES+): m/z 390.2 [M+H]⁺.

### Step 6

Into a 10 mL single-neck round-bottom flask containing a well-stirred solution of 3-[4-(7,11-dioxa-2-azadispiro[3.1.5⁶.1⁴]dodecan-2-yl)-3-fluoro-anilino]piperidine-2,6-dione (7, 200 mg, 513.58 mol) in DCE (3 mL) was added trifluoroacetic acid (2.57 mmol, 196.51 L) slowly at 0° C. under nitrogen atmosphere. The reaction mixture was stirred at 70° C. for 4 hours. After completion of the reaction, the reaction mixture were concentrated under reduced pressure and washed with methyl tert-butyl ether to remove the non-polar impurity to afford 3-[3-fluoro-4-(6-oxo-2-azaspiro[3.3]heptan-2-yl)anilino]piperidine-2,6-dione (B-14, 160 mg, 201.18 mol, 39% yield, TFA salt) as a brown jelly liquid. UPLC-MS (ES+): m/z 332.3 [M+H]⁺.

### Synthesis B15: Synthesis of 1-[3-(2,4-dioxohexahydropyrimidin-1-yl)-5-fluoro-1-methyl-indazol-6-yl]azetidine-3-carbaldehyde (B-15)

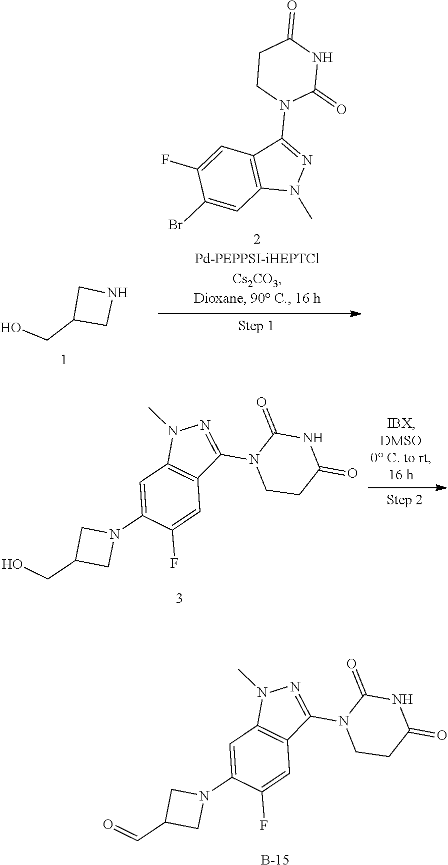

### Step 1

Into a 100 mL sealed tube containing a well-stirred solution of 1-(6-bromo-5-fluoro-1-methyl-indazol-3-yl)hexahydropyrimidine-2,4-dione (2, 1.0 g, 2.93 mmol) and azetidin-3-ylmethanol (1, 510.76 mg, 5.86 mmol) in 1,4-dioxane (20 mL) was added Cs₂CO₃ (2.87 g, 8.79 mmol) at room temperature under nitrogen atmosphere. The reaction mixture was purged with nitrogen gas for 10 minutes. Subsequently, Pd-PEPPSI-iHeptCl (227.94 mg, 234.51 mol) was added and the reaction mixture was stirred at 90° C. for 16 hours. After completion of the reaction, the reaction mixture was filtered through celite pad and washed with 10% methanol in dichloromethane (100 mL). The filtrate was concentrated under reduced pressure to get a crude residue, which was purified by flash column chromatography (50 g, neutral alumina) with 0-15% methanol in dichloromethane, while the desired product was eluted at 5-8% of the mobile phase to afford 1-[5-fluoro-6-[3-(hydroxymethyl)azetidin-1-yl]-1-methyl-indazol-3-yl]hexahydropyrimidine-2,4-dione (3, 400 mg, 1.04 mmol, 35% yield) as a pale yellow solid. UPLC-MS (ES+): m/z 348.1 [M+H]⁺.

### Step 2

Into a 50 mL single neck round bottom flask containing a well stirred solution of 1-[5-fluoro-6-[3-(hydroxymethyl)azetidin-1-yl]-1-methyl-indazol-3-yl]hexahydropyrimidine-2,4-dione (3, 400 mg, 1.15 mmol) in anhydrous DMSO (10 mL) was added IBX (2.15 g, 3.45 mmol, 45% purity) at 0° C. under nitrogen atmosphere. The resulting reaction mixture was allowed to room temperature and stirred for 16 hours. After completion of the reaction, the reaction mixture was diluted with ethyl acetate (150 mL) and washed with saturated NaHCO₃ (3×100 mL) and saturated NaCl solution (50 mL). The organic layer was dried over anhydrous Na₂SO₄ and concentrated under reduced pressure to get the crude 1-[3-(2,4-dioxohexahydropyrimidin-1-yl)-5-fluoro-1-methyl-indazol-6-yl]azetidine-3-carbaldehyde (B-15, 280 mg, 541.95 mol, 47% yield) as a brown gum, which was directly used for the next step without purification. UPLC-MS (ES+): m/z 344.1 [M+H]⁺.

### Synthesis B17: Synthesis of tert-butyl N-[3-fluoro-4-(9-oxo-3-oxabicyclo[3.3.1]nonan-7-yl)phenyl]carbamate (B-17)

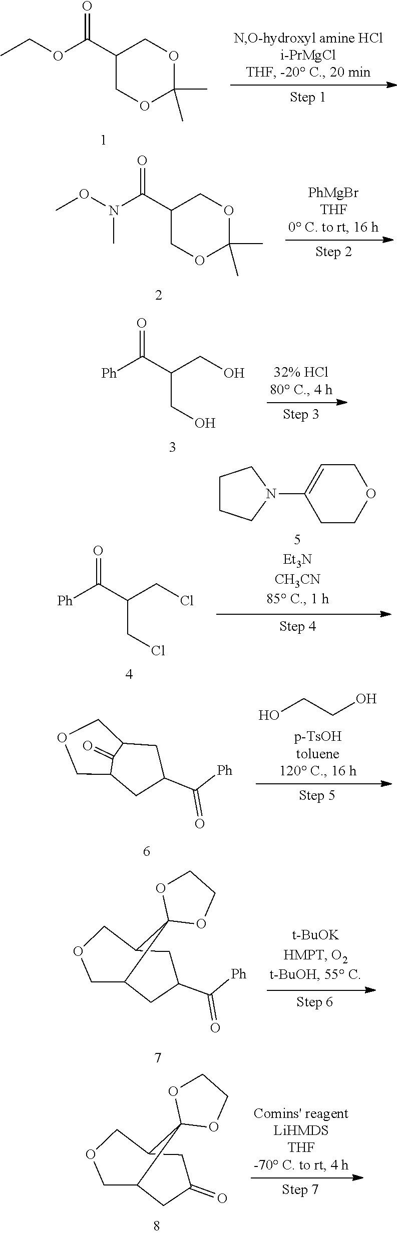

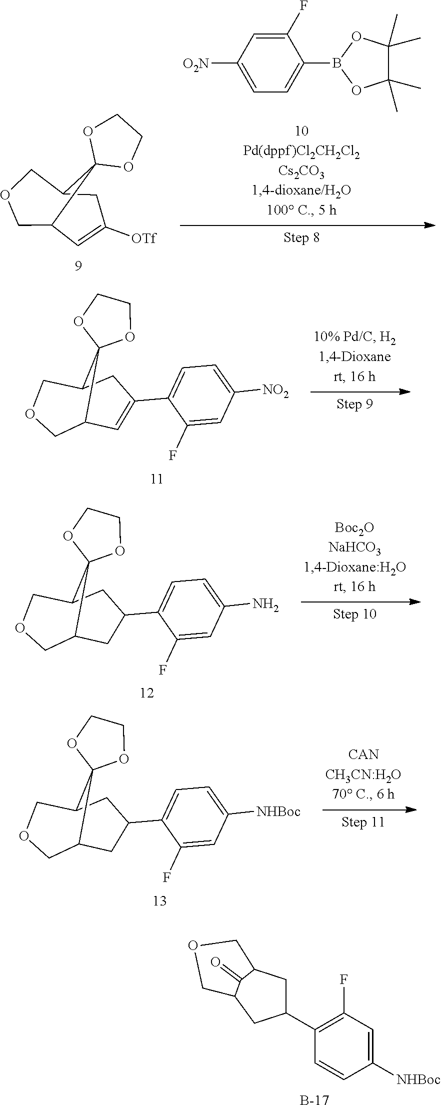

### Step 1

Into a 500 mL three-neck round-bottom flask containing a well-stirred solution of ethyl 2,2-dimethyl-1,3-dioxane-5-carboxylate (1, 12.5 g, 66.41 mmol) and N,O-dimethyl hydroxylamine hydrochloride (10.04 g, 102.94 mmol) in THF (135 mL) was cooled to −20° C., isopropyl magnesium chloride (2 M, 99.73 mL) was added below −5° C. Resulting mixture was stirred at −20° C. for 20 min. After the completion of the reaction, the reaction mixture was quenched with aqueous ammonium chloride (150 mL) and extracted with methyl tert-butyl ether (3×200 mL). The combined organic layers were washed with saturated NaCl (100 mL), dried over anhydrous Na₂SO₄, filtered and concentrated under reduced pressure to afford N-methoxy-N,2,2-trimethyl-1,3-dioxane-5-carboxamide (2, 12.6 g, 59.72 mmol, 90% yield) as a pale-yellow liquid. UPLC-MS (ES+): m/z 204.2 [M+H]⁺.

### Step 2

Into a 500 mL three-neck round-bottom flask containing a well stirred solution of N-methoxy-N,2,2-trimethyl-1,3-dioxane-5-carboxamide (2, 12.6 g, 59.72 mmol) in THF (185 mL) was added phenyl magnesium bromide (1 M in THF, 179.17 mL) drop wise at 0° C. and the resulting reaction mixture was stirred at room temperature for 16 hours. After the completion of the reaction, the reaction mixture was quenched with 1.5 N HCl solution (500 mL) at 0° C. and stirred for 30 min. The resulting suspension was extracted with ethyl acetate (3×400 mL) and the combined organic layers were washed with saturated NaCl (200 mL), dried over anhydrous Na₂SO₄, filtered and concentrated under reduced pressure to afford crude residue which was purified by flash column chromatography (silica gel, 230-400 mesh) to afford 3-hydroxy-2-(hydroxymethyl)-1-phenyl-propan-1-one (3, 10 g, 51.85 mmol, 87% yield) as an off-white solid. UPLC-MS (ES+): m/z 181.0 [M+H]⁺.

### Step 3

Into a 500 mL sealed tube containing 3-hydroxy-2-(hydroxymethyl)-1-phenyl-propan-1-one (3, 15 g, 77.78 mmol) was added HCl (23.71 g, 208.09 mmol, 200 mL, 32%) at 0° C. Resulting mixture was stirred at 80° C. for 4 hours. After completion of the reaction, the reaction mixture was extracted with dichloromethane (3×200 mL). Combined organic layer was washed with saturated NaCl (50 mL), dried over anhydrous sodium sulfate, filtered and concentrated under reduced pressure to afford 3-chloro-2-(chloromethyl)-1-phenyl-propan-1-one (4, 15 g, 54.18 mmol, 70% yield) as a pale-yellow liquid. ¹H NMR (400 MHz, DMSO-d₆): δ 8.03 (d, J=1.20 Hz, 2H), 7.72-7.69 (m, 1H), 7.60-7.56 (m, 2H), 4.49-4.43 (q, 1H), 4.06-4.01 (m, 2H), 3.93-3.88 (m, 2H).

### Step 4

Into a 500 mL single-neck round-bottom flask containing a well stirred solution of 3-chloro-2-(chloromethyl)-1-phenyl-propan-1-one (4, 15 g, 54.18 mmol) in acetonitrile (200 mL) was added triethylamine (81.28 mmol, 11.33 mL) at 0° C. Subsequently, 1-(3,6-dihydro-2H-pyran-4-yl)pyrrolidine (5, 9.96 g, 65.02 mmol) was added and resulting mixture was stirred at 85° C. for 1 hour. After completion, the reaction mixture was quenched with water (150 mL), stirred for 1 h at room temperature and extracted with ethyl acetate (3×300 mL). Combined organic layer was washed with saturated NaCl (150 mL), dried over anhydrous Na₂SO₄, filtered and concentrated under reduced pressure to get crude residue which was purified by flash column chromatography (silica gel, 230-400 mesh), while desired compound was eluting with 10-15% ethyl acetate-hexane to afford 7-benzoyl-3-oxabicyclo[3.3.1]nonan-9-one (6, 11 g, 33.80 mmol, 62% yield) as an off-white solid. UPLC-MS (ES+): m/z 245.0 [M+H]⁺.

### Step 5

Into a 500 mL single-neck round-bottom flask containing a well stirred solution of 7-benzoyl-3-oxabicyclo[3.3.1]nonan-9-one (6, 11 g, 33.80 mmol) in toluene (170 mL) was added ethylene glycol (50.70 mmol, 2.83 mL) andp-toluene sulfonic acid monohydrate (321.46 mg, 1.69 mmol) at room temperature. Resulting mixture was stirred at 120° C. for 16 hours. After completion, reaction mixture was allowed to room temperature, diluted with ethyl acetate (200 mL) and washed with saturated NaHCO₃ solution (100 mL), saturated NaCl (100 mL), dried over anhydrous Na₂SO₄, filtered and concentrated under reduced pressure to get crude which was purified by flash column chromatography (silica gel, 230-400 mesh) to afford phenyl(spiro[1,3-dioxolane-2,9′-3-oxabicyclo[3.3.1]nonane]-7′-yl)methanone (7, 11.5 g, 32.61 mmol, 96% yield) as an off-white solid. UPLC-MS (ES+): m/z 289.0 [M+H]⁺.

### Step 6

Into a 250 mL two-neck round-bottom flask containing a well stirred solution of phenyl(spiro[1,3-dioxolane-2,9′-3-oxabicyclo[3.3.1]nonane]-7′-yl)methanone (7, 4 g, 13.87 mmol) in t-BuOH (54 mL) were added potassium tert-butoxide (1.87 g, 16.65 mmol) and hexamethylphosphoric acid triamide (310.38 mmol, 54.00 mL) at room temperature. Reaction mixture was stirred at 55° C. under oxygen atmosphere (balloon pressure) for 16 hours. After completion the reaction mixture was allowed to room temperature, quenched with saturated NH₄C₁ solution (100 mL) and extracted with ethyl acetate (2×200 mL). The combined organic layer was washed with water (2×100 mL), dried over anhydrous Na₂SO₄, filtered and concentrated under reduced pressure to get crude residue which was purified by flash column chromatography (silica gel 230-400 mesh) by using 0-100% ethyl acetate in hexane while pure compound was eluted at 30-50% of mobile phase to afford spiro[1,3-dioxolane-2,9′-3-oxabicyclo[3.3.1]nonane]-7′-one (8, 500 mg, 2.27 mmol, 16% yield) as an off-white solid. ¹H NMR (400 MHz, DMSO-d₆): δ 4.05-3.99 (m, 4H), 3.70 (d, J=0.80 Hz, 4H), 2.67 (dd, J=5.60, 16.40 Hz, 2H), 2.26-2.22 (m, 2H), 2.00-1.99 (m, 2H).

### Step 7

Into a 100 mL two-neck round-bottom flask containing a well-stirred solution of spiro[1,3-dioxolane-2,9′-3-oxabicyclo[3.3.1]nonane]-7′-one (8, 500 mg, 2.52 mmol) in THF (15 mL) was added LiHMDS (1 M in THF, 7.57 mL) at −40° C. and stirred at the same temperature for 0.5 hours. The reaction mixture was then cooled to −70° C. and a solution of N-(5-chloro-2-pyridyl)-1,1,1-trifluoro-N-(trifluoromethylsulfonyl)methanesulfonamide (2.48 g, 6.31 mmol) in THF (4 mL) was added. The resulting mixture was stirred at room temperature for 4 hours. After completion, the reaction mixture was quenched with saturated NH₄C₁ solution (30 mL) and extracted with ethyl acetate (3×50 mL). The combined organic layer was washed with saturated NaCl (20 mL), dried over anhydrous Na₂SO₄, filtered and concentrated under reduced pressure to get crude which was purified by flash column chromatography (silica gel, 230-400 mesh) to afford spiro[1,3-dioxolane-2,9′-3-oxabicyclo[3.3.1]non-6-ene]-7′-yl trifluoromethanesulfonate (9, 600 mg, 1.71 mmol, 68% yield) as a pale yellow liquid. UPLC-MS (ES−): m/z 329.0 [M−H]⁻.

### Step 8

Into a 40 mL pressure vial containing a well-stirred solution of spiro[1,3-dioxolane-2,9′-3-oxabicyclo[3.3.1]non-6-ene]-7′-yl trifluoromethanesulfonate (9, 600 mg, 1.82 mmol) in 1,4-dioxane (15 mL) and water (5 mL) was added 2-(2-fluoro-4-nitro-phenyl)-4,4,5,5-tetramethyl-1,3,2-dioxaborolane (10, 582.19 mg, 2.18 mmol) and Cs₂CO₃ (1.48 g, 4.54 mmol). The reaction mixture was degassed with nitrogen gas for 10 min followed by addition of Pd(dppf)Cl₂-dichloromethane (148.36 mg, 181.67 mol). The resulting reaction mixture was stirred at 100° C. for 5 hours. After completion, the reaction mixture was allowed to room temperature and diluted with water (30 mL) and extracted with ethyl acetate (2×30 mL). The combined organic layer was washed with saturated NaCl (30 mL), dried over anhydrous Na₂SO₄, filtered and concentrated under reduced pressure to get crude which was purified by flash column chromatography (silica gel, 230-400 mesh) to afford 7′-(2-fluoro-4-nitro-phenyl)spiro[1,3-dioxolane-2,9′-3-oxabicyclo[3.3.1]non-6-ene](11, 580 mg, 1.81 mmol, 99% yield) as a pale brown solid. ¹H NMR (400 MHz, DMSO-d₆): δ 8.11-8.05 (m, 2H), 7.67 (t, J=8.00 Hz, 1H), 6.11 (d, J=6.40 Hz, 1H), 4.06-3.93 (m, 4H), 3.78-3.72 (m, 3H), 3.58-3.55 (m, 1H), 2.89-2.85 (m, 1H), 2.58-2.57 (m, 1H), 2.42-2.40 (m, 1H), 1.99-1.92 (m, 1H).

### Step 9

Into a 25 mL single-neck round-bottom flask containing a well stirred solution of 7′-(2-fluoro-4-nitro-phenyl)spiro[1,3-dioxolane-2,9′-3-oxabicyclo[3.3.1]non-6-ene](11, 510 mg, 1.59 mmol) in 1,4-Dioxane (10 mL) was added 10% palladium on Carbon (600 mg, 1.59 mmol) at room temperature under nitrogen atmosphere. The resulting reaction mixture was stirred at room temperature under hydrogen atmosphere (bladder) for 16 hours. After completion, the reaction mixture was filtered through Celite bed, washed with 1,4-dioxane (30 mL) and 1:1 ethyl acetate/dichloromethane (100 mL). Combined filtrate was concentrated under reduced pressure to get 3-fluoro-4-spiro[1,3-dioxolane-2,9′-3-oxabicyclo[3.3.1]nonane]-7′-yl-aniline (12, 410 mg, 1.31 mmol, 83% yield) as a pale brown solid. UPLC-MS (ES+): m/z 294.1 [M+H]⁺.

### Step 10

Into a 25 mL single-neck round-bottom flask containing a well stirred solution of 3-fluoro-4-spiro[1,3-dioxolane-2,9′-3-oxabicyclo[3.3.1]nonane]-7′-yl-aniline (12, 360 mg, 1.23 mmol) in 1,4-dioxane (3 mL), water (1 mL) was added NaHCO₃ (309.31 mg, 3.68 mmol) and di-tert-butyl dicarbonate (401.78 mg, 1.84 mmol, 422.48 L). The resulting mixture was stirred at room temperature for 16 hours. After completion, the reaction mixture was diluted with water (50 mL) and extracted with ethyl acetate (2×30 mL). The combined organic layer was washed with saturated NaCl (50 mL), dried over anhydrous Na₂SO₄, filtered and concentrated under reduced pressure to get crude which was purified by flash column chromatography (silica gel, 230-400 mesh) to afford tert-butyl N-(3-fluoro-4-spiro[1,3-dioxolane-2,9′-3-oxabicyclo[3.3.1]nonane]-7′-yl-phenyl)carbamate (13, 330 mg, 813.58 mol, 66% yield) as an off-white solid. UPLC-MS (ES+): m/z 338.3 [M+H^{t}Bu]⁺.

### Step 11

Into a 15 mL glass vial containing a well stirred solution of tert-butyl N-(3-fluoro-4-spiro[1,3-dioxolane-2,9′-3-oxabicyclo[3.3.1]nonane]-7′-yl-phenyl)carbamate (13, 330 mg, 838.74 mol) in acetonitrile (6 mL), water (2 mL) was added Cerium(IV) ammonium nitrate (39.27 mg, 83.87 mol) and the resulting mixture was stirred at 70° C. for 6 hours. After completion, the reaction mixture was diluted with water (30 mL) and extracted with ethyl acetate (2×20 mL). Combined organic layer was washed with saturated NaCl (30 mL), dried over anhydrous Na₂SO₄, filtered and concentrated under reduced pressure to get a crude which was purified by flash column chromatography (silica gel, 230-400 mesh), while desired product was eluting with 50-60% ethyl acetate in hexane to afford tert-butyl N-[3-fluoro-4-(9-oxo-3-oxabicyclo[3.3.1]nonan-7-yl)phenyl]carbamate (B-17, 170 mg, 423.30 mol, 50% yield) as an off-white solid. UPLC-MS (ES+): m/z 250.0 [M+H-Boc]⁺.

### Synthesis B18: Synthesis of 1-[5-fluoro-1-methyl-6-(3-oxocyclobutyl)indazol-3-yl]hexahydropyrimidine-2,4-dione (B-18)

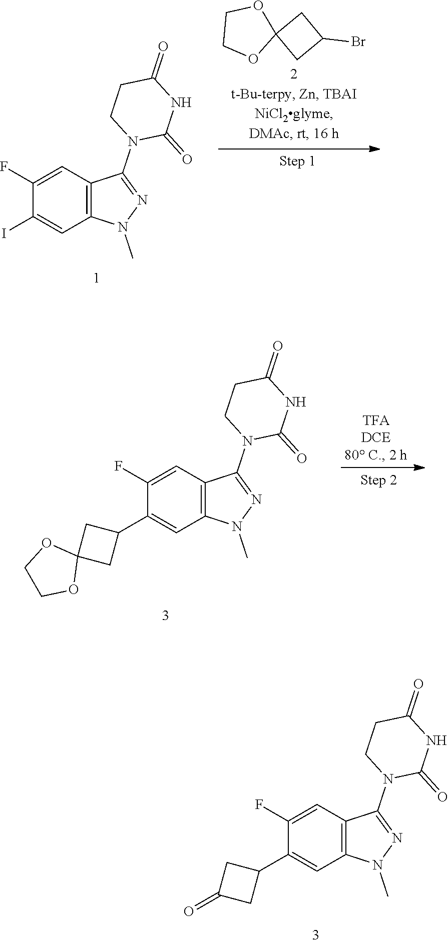 [SMILES: Cn1nc(N2CCC(=O)NC2=O)c2cc(F)c(I)cc21 ; BrC1CC2(C1)OCCO2 ; Cn1nc(N2CCC(=O)NC2=O)c2cc(F)c(C3CC4(C3)OCCO4)cc21 ; Cn1nc(N2CCC(=O)NC2=O)c2cc(F)c(C3CC(=O)C3)cc21]

Into a 40 mL pressure vial containing a well stirred solution of 1-(5-fluoro-6-iodo-1-methyl-indazol-3-yl)hexahydropyrimidine-2,4-dione (1, 600 mg, 1.55 mmol) in anhydrous DMAc (3 mL) were added 2-bromo-5,8-dioxaspiro[3.4]octane (2, 447.61 mg, 2.32 mmol), zinc (303.25 mg, 4.64 mmol) and tetrabutylammonium iodide (171.30 mg, 463.76 μmol) at room temperature under nitrogen atmosphere and mixture was purged with nitrogen gas for 5 min. To the above mixture NiCl₂.glyme (67.93 mg, 309.17 μmol) was added and purging of nitrogen gas was continued for additional 2-3 min. Subsequently, 4,4′,4″-Tri-tert-butyl-2,2′:6′,2″-terpyridine (124.16 mg, 309.17 μmol) was added and the resulting reaction mixture was stirred at room temperature for 16 hours. After completion of the reaction, the reaction mixture was filtered through celite bed and filtrate was concentrated under reduced pressure to get a crude residue which was purified by reverse phase column chromatography (Column: Redisep R_{f} Gold @reversed-phase C18-teledyne ISCO, 120 g, Mobile Phase A: 10 mM ammonium bicarbonate in milli-Q water; Mobile Phase B: acetonitrile). The obtained pure fractions were lyophilized to afford 1-[6-(5,8-dioxaspiro[3.4]octan-2-yl)-5-fluoro-1-methyl-indazol-3-yl]hexahydropyrimidine-2,4-dione (3, 100 mg, 253.76 μmol, 16%) as an off-white solid. UPLC-MS (ES+): m/z 374.9 [M+H]⁺.

### Step 2

Into a 25 mL single-neck round bottom flask containing a well stirred solution of 1-[6-(5,8-dioxaspiro[3.4]octan-2-yl)-5-fluoro-1-methyl-indazol-3-yl]hexahydropyrimidine-2,4-dione (3, 100 mg, 253.76 μmol) in anhydrous 1,2-dichloroethane (1.5 mL) was added trifluoroacetic acid (58.26 μL, 761.29 μmol) at 0° C. and the reaction mixture was stirred at 80° C. for 2 hours. After completion of the reaction, solvents were removed under reduced pressure to get a crude residue which was triturated with methyl tert-butyl ether (2 mL) to afford 1-[5-fluoro-1-methyl-6-(3-oxocyclobutyl)indazol-3-yl]hexahydropyrimidine-2,4-dione (B-18, 50 mg, 148.80 μmol, 59% yield, TFA salt) as an off-white solid. UPLC-MS (ES+): m/z 331.0 [M H]⁺.

### Synthesis B19: Synthesis of 3-[4-[(4S)-3,3-difluoro-4-piperidyl]-3-fluoro-anilino]piperidine-2,6-dione (B-19)

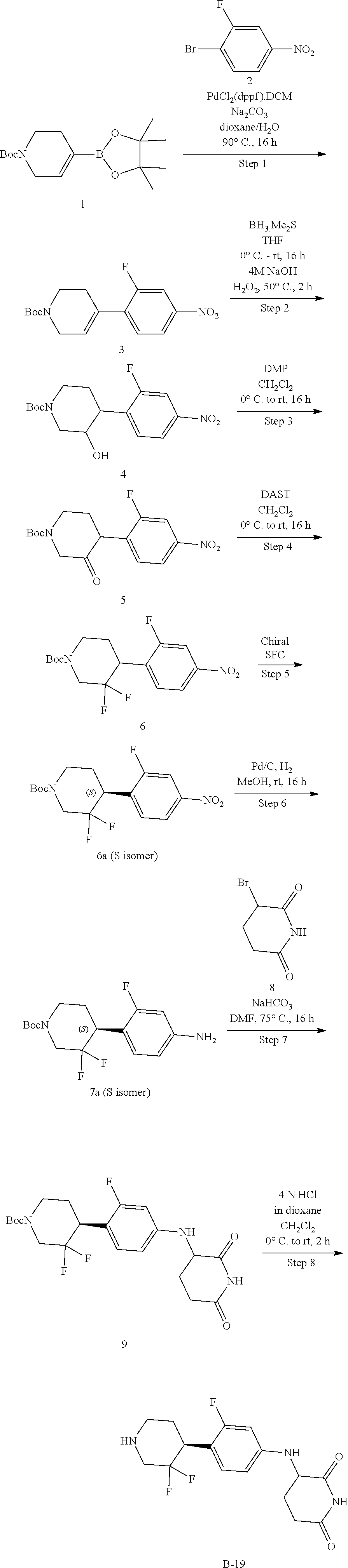

### Step 1

Into a 500 mL three neck round bottom flask containing a well stirred solution of 1-bromo-2-fluoro-4-nitro-benzene (1, 15 g, 68.18 mmol) in 1,4-dioxane (98.18 mL) and water (29.45 mL) were added sodium carbonate (18.07 g, 170.46 mmol) and tert-butyl 4-(4,4,5,5-tetramethyl-1,3,2-dioxaborolan-2-yl)-3,6-dihydro-2H-pyridine-1-carboxylate (2, 23.19 g, 75.00 mmol). The reaction mixture was purged with nitrogen for 10 min. [1,1′-Bis(diphenylphosphino)ferrocene]dichloropalladium(II) dichloromethane (2.78 g, 3.41 mmol) was added and stirred at 90° C. for 16 hours. After completion, the reaction mixture was cooled to room temperature, dissolved in ethyl acetate (500 mL), washed with ethyl acetate, dried over Na₂SO₄, filtered and concentrated under reduced pressure to obtain crude compound which was purified by column chromatography, while desired product eluted at 10% ethyl acetate in pet-ether to obtain tert-butyl 4-(2-fluoro-4-nitro-phenyl)-3,6-dihydro-2H-pyridine-1-carboxylate (3, 15 g, 45.87 mmol, 67% yield). UPLC-MS (ES−): m/z 321.0 [M−H]⁻.

### Step 2

Into a 100 mL three neck round bottom flask containing a well stirred solution of tert-butyl 4-(2-fluoro-4-nitro-phenyl)-3,6-dihydro-2H-pyridine-1-carboxylate (3, 5 g, 15.51 mmol) in anhydrous THF (69.70 mL) was slowly added Borane dimethyl sulfide complex (31.02 mmol, 2.94 mL) at 0° C. The reaction mixture was stirred at room temperature for 16 hours. The reaction mixture was cooled to 0° C. and 4M NaOH solution (1.86 g, 46.54 mmol) was added slowly over a period of 2 hours. After stirring for 30 min 30% hydrogen peroxide (1.58 g, 46.54 mmol) was added. The reaction mixture was stirred at 50° C. for 2 hours. After completion of the reaction, the reaction mixture was cooled to room temperature, poured into ice cold water (25 mL), extracted with ethyl acetate (2×50 mL) and washed with 10% sodium thiosulphate (50 mL) solution. The reaction mixture was dried over Na₂SO₄, filtered and concentrated under reduced pressure to obtain the crude residue which was purified by column chromatography (230-400 silica), while the desired product eluted at 18% ethyl acetate in Hexane to obtain tert-butyl 4-(2-fluoro-4-nitro-phenyl)-3-hydroxy-piperidine-1-carboxylate (4, 4 g, 8.78 mmol, 57% yield) as a pale brown solid. ¹H NMR (400 MHz, DMSO-d₆): δ 8.08-8.01 (m, 2H), 7.80-7.66 (m, 1H), 5.12-4.99 (m, 1H), 4.26-4.11 (m, 1H), 3.91-3.81 (m, 1H), 3.69-3.50 (m, 1H), 3.04-2.84 (m, 1H), 2.82-2.73 (m, 1H), 1.78-1.68 (m, 1H), 1.65-1.55 (m, 2H), 1.36 (s, 9H).

### Step 3

Into a 500 mL three neck round bottom flask containing a well stirred solution of tert-butyl 4-(2-fluoro-4-nitro-phenyl)-3-hydroxy-piperidine-1-carboxylate (4, 8 g, 23.51 mmol) in dichloromethane (200 mL) was added Dess-Martin Periodinane (14.95 g, 35.26 mmol) at 0° C. over a period of 15 min. The reaction mixture was stirrer at room temperature for 16 hours. After completion of the reaction, the reaction mixture was diluted with dichloromethane (100 mL), cooled to 0° C., slowly added 10% NaHCO₃ solution (50 mL), separated organic layers were dried over Na₂SO₄, filtered and concentrated under reduced pressure to obtain crude residue which was purified by column chromatography (230-400 mesh silica) to obtain tert-butyl 4-(2-fluoro-4-nitro-phenyl)-3-oxo-piperidine-1-carboxylate (5, 2.5 g, 4.81 mmol, 20% yield) as a pale brown solid. UPLC-MS (ES+): m/z 338.8 [M+H]⁺.

### Step 4/5

Into a 250 mL two neck round bottom flask containing a well stirred solution of tert-butyl 4-(2-fluoro-4-nitro-phenyl)-3-oxo-piperidine-1-carboxylate (5, 5 g, 14.78 mmol) in dichloromethane (100 mL) was added Diethylaminosulfur trifluoride (32.51 mmol, 4.30 mL) slowly at 0° C. over a period of 10 min. The reaction mixture was stirred at room temperature for 16 hours. After completion of the reaction, the reaction mixture was cooled to 0° C., slowly added 10% NaHCO₃ solution (50 mL), extracted with dichloromethane (2×100 mL), dried over Na₂SO₄ filtered and concentrated under reduced pressure to obtain crude residue which was purified by column chromatography (230-400 mesh silica), while desired compound eluted at 13% ethyl acetate in Hexane to obtain 2.2 g of desired product (6) as a pale-yellow semi solid. Mixture was separated by Chiral SFC following the below method.

### Method of Analysis for chiral SFC separation

Instrument: PIC-22-017
Column: LUXA1-(250*30) mm, 5 m
Mobile Phase: CO₂: 0.2% FA in IPA: CAN (1:1) [75:25]
Total Flow: 100 mL/min
Back pressure: 100 bar
Wavelength: 220 nm
Cycle time: 6 min

First eluted fractions were lyophilized to afford peak-1 (S-isomer) tert-butyl (4S)-3,3-difluoro-4-(2-fluoro-4-nitro-phenyl)piperidine-1-carboxylate (6a, 1 g, 2.74 mmol, 19% yield) as a pale brown semi solid. UPLC-MS (ES+): m/z 261.2 [M−Boc+H]⁺.

### Step 6

Into a 50 mL single neck round-bottom flask containing a well stirred solution of tert-butyl (4S)-3,3-difluoro-4-(2-fluoro-4-nitro-phenyl)piperidine-1-carboxylate (6a, 1 g, 2.78 mmol) in methanol (20 mL) was added palladium on activated charcoal (295.34 mg, 2.78 mmol) at room temperature. The reaction mixture was stirred at room temperature under hydrogen bladder pressure for 16 hours. After completion of the reaction, the reaction mixture was filtered through celite pad and washed with methanol (20 mL), concentrated under reduced pressure to afford tert-butyl (4S)-4-(4-amino-2-fluoro-phenyl)-3,3-difluoro-piperidine-1-carboxylate (7a, 0.8 g, 2.31 mmol, 83% yield) as a pale brown semi solid. UPLC-MS (ES+): m/z 275.2 [M-^{t}Bu+H]⁺.

### Step 7

Into a 50 mL sealed tube containing a well stirred solution of tert-butyl (4S)-4-(4-amino-2-fluoro-phenyl)-3,3-difluoro-piperidine-1-carboxylate (7a, 0.8 g, 2.42 mmol) in N,N-dimethylformamide (4.81 mL) were added 3-bromopiperidine-2,6-dione (8, 2.32 g, 12.11 mmol) and NaHCO₃ (1.63 g, 19.37 mmol) at room temperature under nitrogen atmosphere. The reaction mixture was stirred at 75° C. for 16 hours. After completion of the reaction, the reaction mixture was cooled to room temperature, poured into ice cold H₂O (50 mL), extracted with ethyl acetate (3×50 mL). The combined organic layers were washed with ice cold H₂O (2×20 mL), dried over Na₂SO₄ filtered and concentrated under reduced pressure to obtain a crude residue which was purified by column chromatography (100-200 mesh silica) to obtain tert-butyl (4S)-4-[4-[(2,6-dioxo-3-piperidyl)amino]-2-fluoro-phenyl]-3,3-difluoro-piperidine-1-carboxylate (9, 500 mg, 1.10 mmol, 45% yield) as a pale brown solid. UPLC-MS (ES−): m/z 440.2 [M−H]⁻.

### Step 8

Into a 50 mL single neck round bottom flask containing a well stirred solution of tert-butyl (4S)-4-[4-[(2,6-dioxo-3-piperidyl)amino]-2-fluoro-phenyl]-3,3-difluoro-piperidine-1-carboxylate (9, 400 mg, 906.12 mol) in dichloromethane (10 mL) was added 4.0M hydrogen chloride in dioxane (4.53 mmol, 206.49 L) slowly at 0° C. The reaction mixture was stirred at room temperature for 2 hours. After completion of the reaction, the reaction mixture was concentrated under reduced pressure to obtain 3-[4-[(4S)-3,3-difluoro-4-piperidyl]-3-fluoro-anilino]piperidine-2,6-dione (B-19, 330 mg, 852.02 mol, 94% yield, HCl salt) as an off-white solid. UPLC-MS (ES+): m/z 342.2 [M+H]⁺.

### Synthesis B20: Synthesis of 1-[5-fluoro-6-(3-hydroxy-4-oxo-cyclohexyl)-1-methyl-indazol-3-yl]hexahydropyrimidine-2,4-dione (B-20)

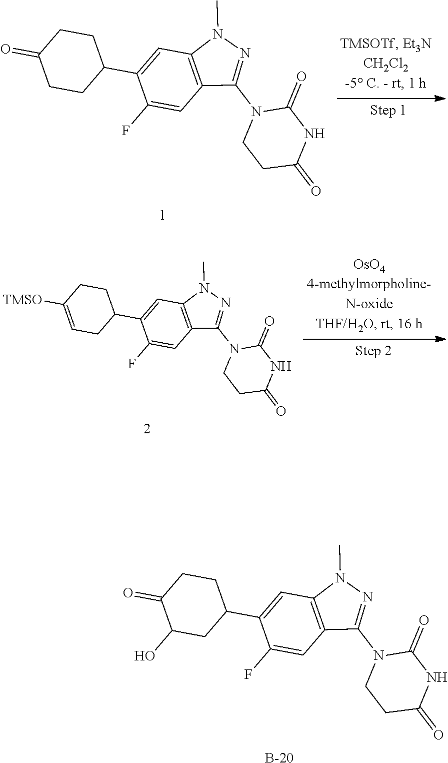

### Step 1

Into a 100 mL two-neck round-bottom flask containing a well-stirred solution of 1-[5-fluoro-1-methyl-6-(4-oxocyclohexyl)indazol-3-yl]hexahydropyrimidine-2,4-dione (1, 1.0 g, 2.79 mmol) and triethylamine (8.37 mmol, 1.17 mL) in dichloromethane (40 mL) was added trimethylsilyl trifluoromethanesulfonate (3.63 mmol, 656.57 μL) in dichloromethane (10 mL) at −5° C. under nitrogen atmosphere. The resulting reaction mixture was stirred at room temperature for 1 hour. After completion of the reaction, the reaction mixture was poured into water (50 mL) and extracted with dichloromethane (3×50 mL). The combined organic layers were dried over anhydrous Na₂SO₄, filtered, and concentrated under reduced pressure to afford 1-[5-fluoro-1-methyl-6-(4-trimethylsilyloxycyclohex-3-en-1-yl)indazol-3-yl]hexahydropyrimidine-2,4-dione (2, 1.2 g, 1.34 mmol, 45% yield) as an off-white semi-solid. UPLC-MS (ES+): m/z 431.0 [M+H]⁺.

### Step 2

Into a 100 mL single-neck round-bottom flask containing a well-stirred solution of 1-[5-fluoro-1-methyl-6-(4-trimethylsilyloxycyclohex-3-en-1-yl)indazol-3-yl]hexahydropyrimidine-2,4-dione (2, 1.2 g, 2.79 mmol) in THF (10 mL) and water (10 mL), was added 4-methylmorpholine-N-oxide (408.14 mg, 3.48 mmol) and 4% OsO₄ in water (1.39 mmol, 354.29 L) at 0° C. under nitrogen atmosphere. The resulting reaction mixture was stirred for 16 h at room temperature. After completion of the reaction, the reaction mixture was concentrated under reduced pressure to get a crude residue which was co-distilled with toluene (3×20 mL). The crude compound was purified by flash column chromatography (25 g, 230-400 silica-gel) using 0-100% ethyl acetate/hexane as an eluent while compound was eluting at 20-50% ethyl acetate/hexane to afford 1-[5-fluoro-6-(3-hydroxy-4-oxo-cyclohexyl)-1-methyl-indazol-3-yl]hexahydropyrimidine-2,4-dione (B-20, 0.320 g, 829.14 mol, 30% yield) as an off-white solid. UPLC-MS (ES+): m/z 375.2 [M+H]⁺.

### Synthesis B21: Synthesis of 1-[6-(2,6-diazaspiro[3.3]heptan-2-yl)-5-fluoro-1-methyl-indazol-3-yl]hexahydropyrimidine-2,4-dione (B-21)

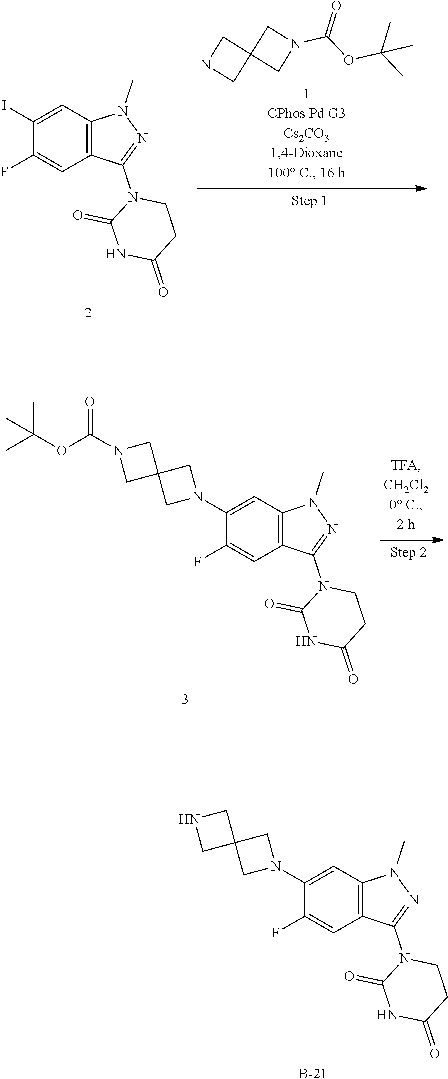

### Step 1

Into a 8 mL pressure capped vial containing a well-stirred solution of tert-butyl 2,6-diazaspiro[3.3]heptane-2-carboxylate (1, 612.97 mg, 3.09 mmol) and 1-(5-fluoro-6-iodo-1-methyl-indazol-3-yl)hexahydropyrimidine-2,4-dione (2, 1 g, 2.58 mmol) in anhydrous 1,4-dioxane (10 mL) was added Cs₂CO₃ (2.52 g, 7.73 mmol) at room temperature under nitrogen atmosphere and the resulting mixture was degassed with nitrogen for 10 minutes. Subsequently, CPhos Pd-G3 (207.75 mg, 257.64 μmol) was added, and reaction mixture was stirred at 100° C. for 16 hours. After completion of the reaction, the reaction mixture was filtered through celite bed and bed was washed with ethyl acetate (50 mL). Filtrate was concentrated under reduced pressure to get a crude residue which was purified by flash silica-gel (230-400 mesh, 25 g) column chromatography eluting with 0-100% ethyl acetate/hexane to afford tert-butyl 6-[3-(2,4-dioxohexahydropyrimidin-1-yl)-5-fluoro-1-methyl-indazol-6-yl]-2,6-diazaspiro[3.3]heptane-2-carboxylate (3, 260 mg, 533.06 μmol, 21% yield) as an off-white solid. UPLC-MS (ES+): m/z 459.2 [M+H]⁺.

### Step 2

Into a 25 mL single-neck round-bottom flask containing a well-stirred solution of tert-butyl 6-[3-(2,4-dioxohexahydropyrimidin-1-yl)-5-fluoro-1-methyl-indazol-6-yl]-2,6-diazaspiro[3.3]heptane-2-carboxylate (3, 259 mg, 531.01 mol) in anhydrous dichloromethane (2 mL) was added trifluoroacetic acid (7.97 mmol, 609.52 μL) at 0° C. and the mixture was stirred at 0° C. for 2 h. After completion of reaction, solvents were removed under reduced pressure to get a crude residue which was triturated with methyl tert-butyl ether (2×2 mL) and dried under reduced pressure to afford 1-[6-(2,6-diazaspiro[3.3]heptan-2-yl)-5-fluoro-1-methyl-indazol-3-yl]hexahydropyrimidine-2,4-dione (B-21, 230 mg, 413.85 μmol, 78% yield, TFA salt) as a brown solid. UPLC-MS (ES+): m/z 359.3 [M+H]⁺.

### Synthesis B22: Synthesis of 3-[3-fluoro-4-(3-oxocyclobutyl)anilino]piperidine-2,6-dione (B-22)

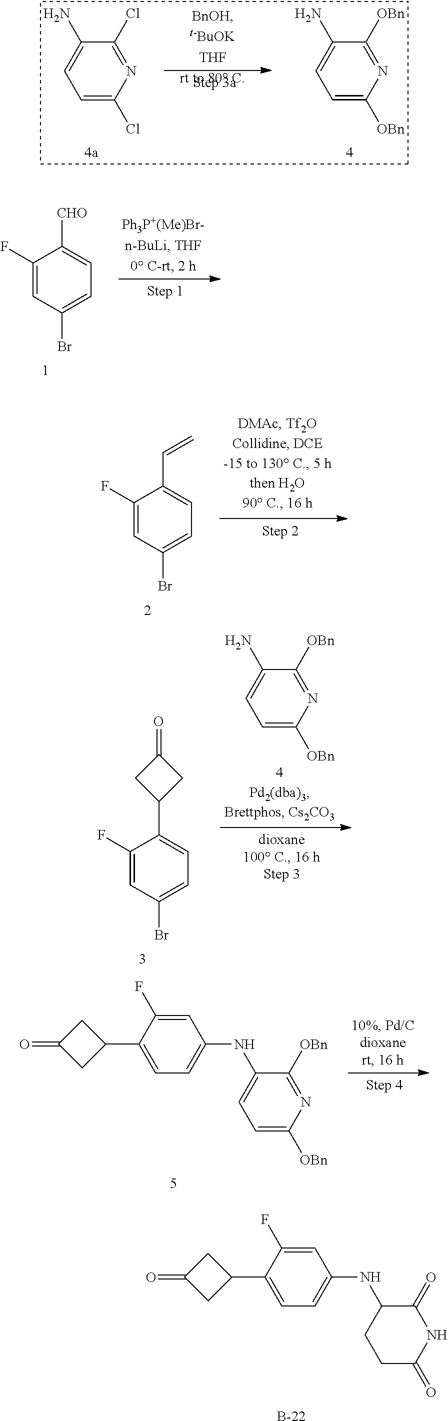

### Step 3a

Into a 250 mL three-neck round-bottom flask containing a well-stirred solution of benzyl Alcohol (6.63 g, 61.35 mmol, 6.35 mL) in anhydrous THF (80 mL), potassium tert-butoxide (6.88 g, 61.35 mmol) was added portion-wise to the reaction mixture after purging nitrogen for 30 min. The reaction mixture was stirred at room temperature for 2 hours. 2,6-Dichloropyridin-3-amine (4a, 4 g, 24.54 mmol) was added and stirred at 80° C. for 24 hours. After completion of the reaction, the reaction mixture was cooled to room temperature and diluted with water (150 mL) and extracted with ethyl acetate (2×150 mL). The combined organic layer were washed with saturated NaCl (100 mL), dried over anhydrous Na₂SO₄, filtered and concentrated under reduced pressure to afford crude residue which was purified by silica gel (100-200 mesh) column chromatography using a gradient of 0-100% ethyl acetate/hexane, while desired product was eluted at 10% ethyl acetate/hexane to afford 2,6-dibenzyloxypyridin-3-amine (4, 2 g, 5.83 mmol, 24% yield) as a dark brown gummy liquid. UPLC-MS (ES+): m/z 307.2 [M+H]⁺.

### Step 1

Into a 500 mL three-neck round-bottom flask containing a well-stirred solution of methyltriphenylphosphonium bromide (17.60 g, 49.26 mmol) in THF (200 mL) was added n-BuLi (2.5 M in hexane, 19.70 mL) slowly at 0° C. for 30 min under nitrogen atmosphere, followed by the addition 4-bromo-2-fluoro-benzaldehyde (1, 10 g, 49.26 mmol) in THF (200 mL) slowly at 0° C. The resulting mixture was stirred at ambient temperature for 2 hours. After completion of the reaction, the reaction mixture was quenched with saturated NH₄C₁ solution (250 mL) and extracted with ethyl acetate (3×500 mL). The combined organic layer were washed with saturated NaCl solution (250 mL), dried over anhydrous Na₂SO₄ and concentrated under reduced pressure to get a crude residue, which was purified by flash silica-gel (230-400 mesh) column chromatography using the gradient of 0-100% ethyl acetate/Hexane while pure compound was eluting at 10-15% of mobile phase to afford 4-bromo-2-fluoro-1-vinyl-benzene (2, 7.0 g, 34.82 mmol, 71% yield) as pale yellow liquid. ¹H NMR (400 MHz, DMSO-d₆): δ 7.71-7.52 (m, 2H), 7.42 (dd, J=2.00, 8.40 Hz, 1H), 6.83-6.75 (m, 1H), 5.97 (dd, J=0.80, 17.60 Hz, 1H), 5.49 (dd, J=1.20, 11.20 Hz, 1H).

### Step 2

Into a 100 mL two-neck round-bottom flask containing a well-stirred solution of N,N-dimethylacetamide (21.89 mmol, 2.03 mL) in DCE (10 mL) was added a solution of trifluoromethane sulphonic anhydride (21.89 mmol, 3.68 mL) in DCE (10 mL) at −15° C. under nitrogen atmosphere and stirred at same temperature for 30 min. Finally, a solution of 4-bromo-2-fluoro-1-vinyl-benzene (2, 4 g, 19.90 mmol) in DCE (10 mL) followed by a solution of 2,4,6-trimethylpyridine (21.89 mmol, 2.90 mL) in DCE (10 mL) was added to the reaction mixture. The reaction mixture was stirred at 130° C. for 5 hours. After 5 h, the reaction was cooled to room temperature, water (5 mL) was added to the reaction mixture and further stirred at 90° C. for 16 hours. After completion of reaction, the reaction mixture was diluted with water (50 mL) and extracted with dichloromethane (3×70 mL) and the combined organic layer were washed with saturated NaCl (50 mL), dried over anhydrous Na₂SO₄, filtered and concentrated under reduced pressure to get a crude residue, which was purified by silica gel (100-200 mesh) column chromatography with the gradient of 0-100% ethyl acetate/Hexane, while desired product was eluted at 10-15% ethyl acetate/hexane to afford 3-(4-bromo-2-fluoro-phenyl)cyclobutanone (3, 1.2 g, 4.84 mmol, 24% yield) as a yellow liquid. ¹H NMR (400 MHz, DMSO-d₆): δ 7.33-7.27 (m, 2H), 7.20-7.16 (m, 1H), 3.82-3.68 (m, 1H), 3.61-3.43 (m, 2H), 3.40-3.20 (m, 2H).

### Step 3

Into a 20 mL pressure vial containing a well-stirred solution of 2,6-dibenzyloxypyridin-3-amine (4, 0.5 g, 1.63 mmol) and 3-(4-bromo-2-fluoro-phenyl)cyclobutanone (3, 436.38 mg, 1.80 mmol) in 1,4 Dioxane (10 mL) was added Cs₂CO₃ (1.60 g, 4.90 mmol) at room temperature under nitrogen atmosphere. The reaction mixture was degassed for 15 min by purging nitrogen. Finally, BrettPhos (175.21 mg, 326.42 mol) and Tris(dibenzylideneacetone)dipalladium(0) (298.91 mg, 326.42 mol) were added and stirred at 100° C. for 16 hours. After completion of reaction, the reaction mixture was cooled to room temperature, diluted with water (50 mL) and extracted with ethyl acetate (2×60 mL). The combined organic layer were washed with saturated NaCl solution (50 mL) and dried over anhydrous Na₂SO₄ and concentrated under reduced pressure to get a crude residue, which was purified by silica gel (100-200 mesh) column chromatography, using a gradient of 0-100% ethyl acetate/hexane to afford 3-[4-[(2,6-dibenzyloxy-3-pyridyl)amino]-2-fluoro-phenyl]cyclobutanone (5, 0.500 g, 781.61 mol, 48% yield) as a dark brown liquid. UPLC-MS (ES+): m/z 469.2 [M+H]⁺.

### Step 4

Into a 100 mL single-neck round-bottom flask containing a well-stirred solution of 3-[4-[(2,6-dibenzyloxy-3-pyridyl)amino]-2-fluoro-phenyl]cyclobutanone (5, 0.5 g, 1.07 mmol) in 1,4 Dioxane (10 mL) was added 10% palladium on carbon (0.4 g, 1.07 mmol) and stirred at room temperature for 16 h under hydrogen pressure. After completion of reaction, the reaction mixture was filtered through celite pad and washed with ethyl acetate (150 mL), the organic layer was evaporated under reduced pressure to afford 3-[3-fluoro-4-(3-oxocyclobutyl)anilino]piperidine-2,6-dione (B-22, 300 mg, 950.78 mol, 89% yield) as brown gummy liquid. UPLC-MS (ES+): m/z 291.0 [M+H]⁺.

### Synthesis B23: Synthesis of 3-(3-fluoro-4-piperazin-1-yl-anilino)piperidine-2,6-dione (B-23)

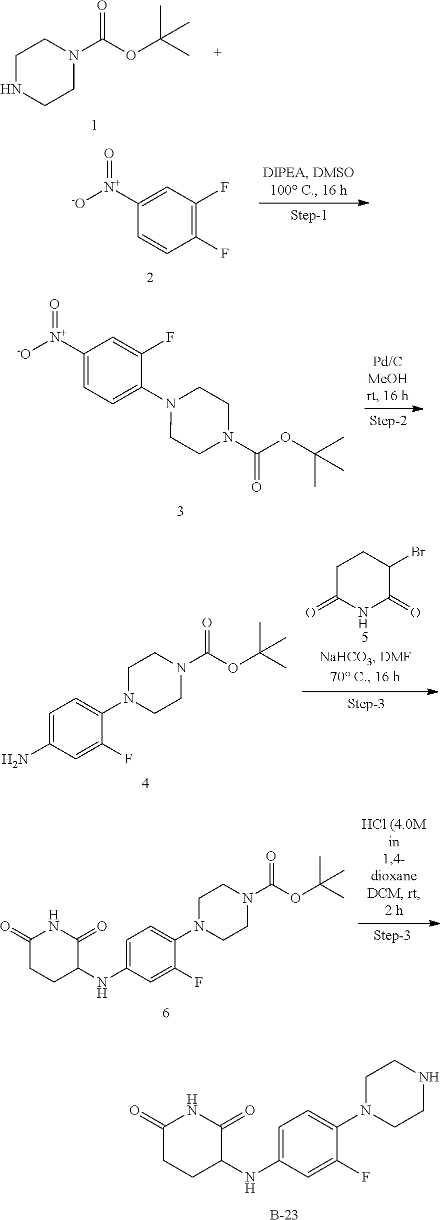

### Step 1

Into a 100 mL two neck round bottom flask containing a stirred solution of tert-butyl piperazine-1-carboxylate (1, 7.02 g, 37.71 mmol) in DMSO (50 mL) were added 1,2-difluoro-4-nitro-benzene (2, 5 g, 31.43 mmol, 3.48 mL) and N,N-diisopropylethylamine (16.42 mL, 94.29 mmol) to reaction mixture and stirred at 100° C. for 16 hours. After completion of the reaction, the reaction mixture was quenched with ice-cold solution and extracted with ethyl acetate (3×50 mL). The combined organic layers were dried over sodium sulphate and concentrated under reduced pressure to get crude residue. The crude compound was purified by (100-200 silica) column chromatography (40% EtOAc/hexane) to afford tert-butyl 4-(2-fluoro-4-nitro-phenyl)piperazine-1-carboxylate (3, 8 g, 24.46 mmol, 78% yield) as yellow solid. ¹H NMR (400 MHz, DMSO-d₆): δ 8.06-8.01 (m, 2H), 7.21-7.16 (m, 1H), 3.50-3.47 (m, 4H), 3.28-3.25 (m, 4H), 1.43 (s, 9H).

### Step 2

Into a 500 mL single neck round bottom flask containing a stirred solution of tert-butyl 4-(2-fluoro-4-nitro-phenyl)piperazine-1-carboxylate (3, 8 g, 24.59 mmol) in methanol (160 mL) at room temperature and resulting mixture was degassed by bubbling nitrogen gas for 10 min. Subsequently, 10% Pd/C (4 g, 32.94 mmol) was added, and the resulting reaction mixture was stirred at room temperature under hydrogen bladder pressure for 16 hours. The reaction mixture was filtered through a celite pad and the celite pad was washed with methanol (150 mL) and mixture of ethyl acetate/dichloromethane (1:1) (200 mL). Combined filtrate was concentrated under reduced pressure to afford a crude tert-butyl 4-(4-amino-2-fluoro-phenyl)piperazine-1-carboxylate (4, 7 g, 18.24 mmol, 74% yield). UPLC-MS: m/z 296.3 [M+H]⁺.

### Step 3

Into a 250 mL glass sealed tube containing a stirred solution of tert-butyl 4-(4-amino-2-fluoro-phenyl)piperazine-1-carboxylate (4, 4 g, 13.54 mmol) in N,N-dimethylformamide (20 mL) were added 3-bromo piperidine-2,6-dione (5, 7.80 g, 40.63 mmol) and NaHCO₃ (5.69 g, 67.72 mmol) to the reaction mixture and stirred at 70° C. for 16 hours. The reaction mixture was cooled to room temperature and quenched with ice cold water (250 mL), added ethyl acetate (250 mL) to the reaction mixture. Reaction mixture was filtered through celite pad, extracted with ethyl acetate (3×70 mL). The combined organic layers were dried over sodium sulphate and concentrated under reduced pressure to get a crude residue. The crude compound was purified by 230-400 silica mesh column chromatography, while desired compound eluted at 30% ethyl acetate/Hexane to obtain tert-butyl 4-[4-[(2,6-dioxo-3-piperidyl)amino]-2-fluoro-phenyl]piperazine-1-carboxylate (6, 3.4 g, 8.19 mmol, 60% yield) as green solid. UPLC-MS: m/z 407.2 [M+H]⁺.

### Step 4

Into a 50 mL single neck round bottom flask containing a stirred solution of tert-butyl 4-[4-[(2,6-dioxo-3-piperidyl)amino]-2-fluoro-phenyl]piperazine-1-carboxylate (6, 0.5 g, 1.23 mmol) in DCM (10 mL) was added HCl (4M in 1,4-Dioxane) (5 mL) at 0° C. The reaction mixture was stirred at room temperature for 2 hours. The solvents were evaporated and triturated with methyl tert-butyl ether (10 mL) to afford crude 3-(3-fluoro-4-piperazin-1-yl-anilino)piperidine-2,6-dione (B-23, 350 mg, 478.65 μmol, 39% yield). ¹H NMR (400 MHz, DMSO-d₆): δ 10.78 (s, 1H), 8.83 (bs, 2H), 6.90-6.86 (m, 1H), 6.56-6.52 (m, 1H), 6.46-6.43 (m, 1H), 4.30-4.26 (m, 1H), 3.28-3.18 (m, 4H), 3.11-3.03 (m, 4H), 2.63-2.52 (m, 2H), 2.44-2.41 (m, 2H).

### Synthesis B24: Synthesis of 1-[5-fluoro-1-methyl-6-(3-oxa-9-azabicyclo[3.3.1]nonan-7-yl)indazol-3-yl]hexahydropyrimidine-2,4-dione (B-24)

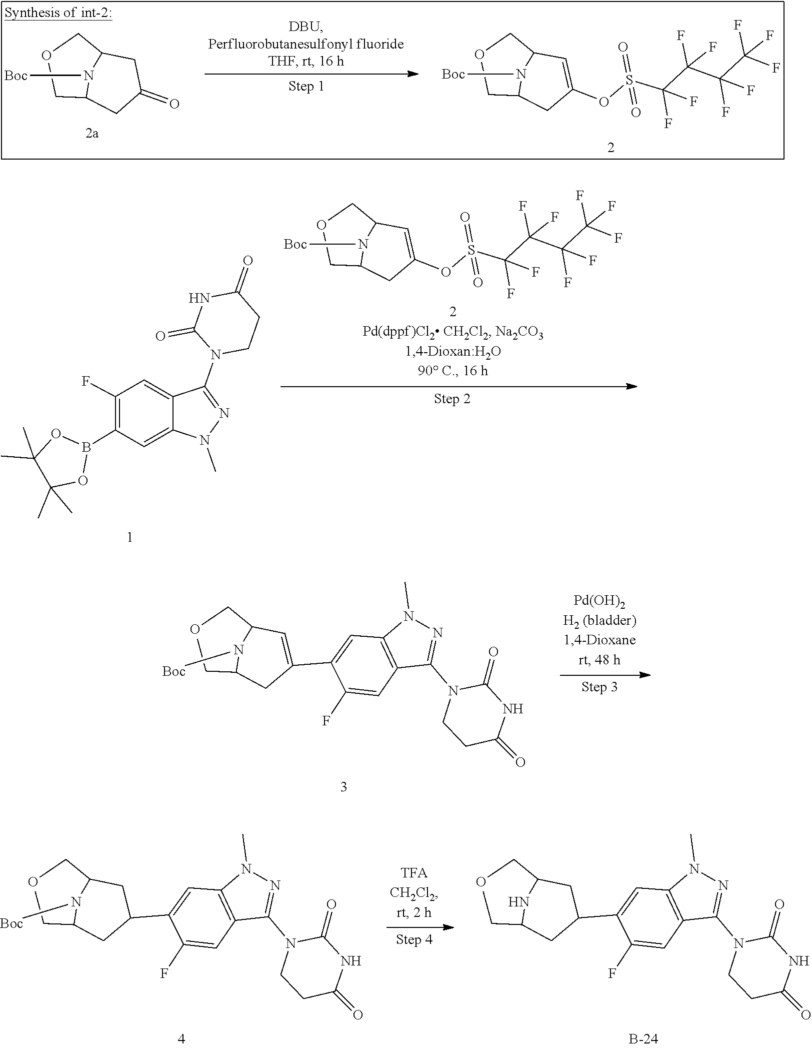

### Step 1

Into a 100 mL two-neck round-bottom flask containing a well-stirred solution of tert-butyl 7-oxo-3-oxa-9-azabicyclo[3.3.1]nonane-9-carboxylate (2a, 2 g, 8.29 mmol) in THF (40 mL) was added DBU (49.73 mmol, 7.44 mL) and perfluorobutanesulfonyl fluoride (49.73 mmol, 8.59 mL) at room temperature under nitrogen atmosphere and stirred at room temperature for 16 hours. The reaction mixture was diluted with ethyl acetate (100 mL) and washed with water (100 mL) and saturated NaCl (100 mL). Organic layer was separated and dried over anhydrous Na₂SO₄ filtered and evaporated under reduced pressure to obtain a crude residue which was purified by flash silica-gel (230-400 mesh) column with 0-100% ethyl acetate/hexane to afford tert-butyl 7-(1,1,2,2,3,3,4,4,4-nonafluorobutylsulfonyloxy)-3-oxa-9-azabicyclo[3.3.1]non-6-ene-9-carboxylate (2, 3.5 g, 6.62 mmol, 80% yield) as an off-white solid. UPLC-MS (ES+). m/z 424.0 [M+H-Boc]⁺.

### Step 2

Into a 40 mL pressure vial containing a well-stirred solution of 1-[5-fluoro-1-methyl-6-(4,4,5,5-tetramethyl-1,3,2-dioxaborolan-2-yl)indazol-3-yl]hexahydropyrimidine-2,4-dione (1, 1 g, 2.58 mmol) in 1,4-dioxane (12 mL) and water (3 mL) were added tert-butyl 7-(1,1,2,2,3,3,4,4,4-nonafluorobutylsulfonyloxy)-3-oxa-9-azabicyclo[3.3.1]non-6-ene-9-carboxylate (2, 1.48 g, 2.83 mmol), sodium carbonate (839.31 mg, 7.92 mmol) at ambient temperature under nitrogen atmosphere and the resulting mixture was degassed for 10 minutes by bubbling nitrogen gas into the reaction mixture. PdCl₂dppf-DCM (210.36 mg, 257.60 mol) was added to the reaction mixture, degassed for 5 min and reaction mixture was stirred at 90° C. for 16 hours. The reaction mixture was cooled to room temperature and poured into water (100 mL) and extracted with ethyl acetate (2×100 mL). Combined organic layer were washed with saturated NaCl (100 mL), dried over anhydrous Na₂SO₄, filtered and filtrate was concentrated under reduced pressure to get a crude residue, which was purified by flash silica-gel (230-400 mesh) column with 0-100% ethyl acetate/petroleum ether to afford tert-butyl 7-[3-(2,4-dioxohexahydropyrimidin-1-yl)-5-fluoro-1-methyl-indazol-6-yl]-3-oxa-9-azabicyclo[3.3.1]non-6-ene-9-carboxylate (3, 520 mg, 996.07 mol, 39% yield) as an off-white solid. UPLC-MS (ES+): m/z 486.0 [M+H]⁺.

### Step 3

Into a 50 mL single-neck round-bottom flask containing a well-stirred solution of tert-butyl 7-[3-(2,4-dioxohexahydropyrimidin-1-yl)-5-fluoro-1-methyl-indazol-6-yl]-3-oxa-9-azabicyclo[3.3.1]non-6-ene-9-carboxylate (3, 590 mg, 1.22 mmol) in 1,4-dioxane (12 mL) was added 20% Pd(OH)₂ (900 mg, 1.22 mmol) [20% by weight and 50% water] at ambient temperature under nitrogen atmosphere. The resulting suspension was stirred at room temperature under hydrogen bladder pressure for 48 hours. After completion of the reaction, the reaction mixture was filtered through a pad of celite and the celite pad was washed with 1,4-dioxane (50 mL) and 1:1 ethyl acetate/dichloromethane (200 mL). Combined organic layer was evaporated under reduced pressure to get crude tert-butyl 7-[3-(2,4-dioxohexahydropyrimidin-1-yl)-5-fluoro-1-methyl-indazol-6-yl]-3-oxa-9-azabicyclo[3.3.1]nonane-9-carboxylate (4, 480 mg, 974.72 mol, 80% yield) as a brown solid. UPLC-MS (ES+): m/z 488.1 [M+H]⁺.

### Step 4

Into a 25 mL single-neck round-bottom flask containing a well-stirred solution of tert-butyl 7-[3-(2,4-dioxohexahydropyrimidin-1-yl)-5-fluoro-1-methyl-indazol-6-yl]-3-oxa-9-azabicyclo[3.3.1]nonane-9-carboxylate (4, 480 mg, 984.57 mol) in anhydrous dichloromethane (5 mL) was added trifluoroacetic acid (9.85 mmol, 753.45 L) at room temperature. The resulting mixture was stirred at room temperature for 2 hours. After completion of the reaction solvents were evaporated under reduced pressure to get a crude residue which was triturated with hexane (10 mL) to remove non-polar impurity and dried under reduced pressure to afford 1-[5-fluoro-1-methyl-6-(3-oxa-9-azabicyclo[3.3.1]nonan-7-yl)indazol-3-yl]hexahydropyrimidine-2,4-dione (B-24, 430 mg, 848.97 mol, 86% yield, TFA salt) as an off-white solid. UPLC-MS (ES+): m/z 388.2 [M+H]⁺.

### Synthesis B25: Synthesis of 2-[l-[3-(2,4-dioxohexahydropyrimidin-1-yl)-5-fluoro-1-methyl-indazol-6-yl]-4-hydroxy-4-piperidyl]acetic acid (B-25)

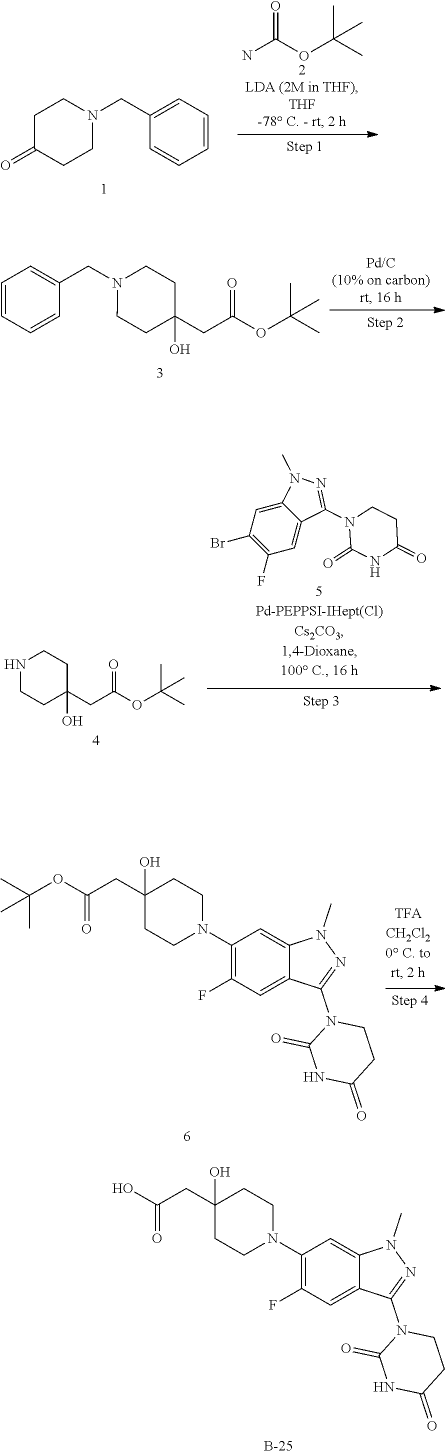

### Step 1

Into a 100 mL three-neck round-bottom flask containing a well-stirred solution of tert-butyl acetate (2, 613.77 mg, 5.28 mmol) in THF (15 mL) was added LDA (2.0 M in THF) (2 M, 5.28 mL) at −78° C. and resulting reaction mixture was stirred at the same temperature for 1 hour. A solution of 1-benzylpiperidin-4-one (1, 1 g, 5.28 mmol) dissolved in THF (3 mL) was added to the reaction mixture at −78° C. and stirred for 2 h at −78° C. Further, reaction mixture was stirred at room temperature for 2 hours. After completion of the reaction, the reaction mixture was quenched with saturated aqueous NH₄Cl solution (20 mL) and extracted with ethyl acetate (2×100 mL). Combined organic layer was washed with water (10 mL), saturated NaCl (10 mL), dried over Na₂SO₄ and filtered, filtrate was concentrated under reduced pressure to get crude residue which purified by silica gel column chromatography (100-200 mesh) using a gradient of 0-100% ethyl acetate/hexane to afford tert-butyl 2-(1-benzyl-4-hydroxy-4-piperidyl)acetate (3, 1.1 g, 3.57 mmol, 67% yield) as light yellow solid. UPLC-MS (ES+): m/z 306.5 [M+H]⁺.

### Step 2

Into a 25 mL tinyclave containing a well-stirred solution of tert-butyl 2-(1-benzyl-4-hydroxy-4-piperidyl)acetate (3, 900 mg, 2.92 mmol) in methanol (10 mL) was added palladium on activated charcoal (wetted with ca. 55% Water) (800 mg, 7.52 mmol) at room temperature. The reaction mixture was stirred at room temperature under hydrogen pressure (4 kg/cm²) for 16 hours. After completion of the reaction, the reaction mixture was filtered through celite pad and washed with methanol (20 mL), filtrate was concentrated under reduced pressure to afford tert-butyl 2-(4-hydroxy-4-piperidyl)acetate (4, 580 mg, 2.62 mmol, 90% yield) as a pale brown semi solid. UPLC-MS (ES+): m/z 216.3 [M+H]⁺.

### Step 3

Into a 100 mL Sealed tube containing a well-stirred solution of tert-butyl 2-(4-hydroxy-4-piperidyl)acetate (4, 350 mg, 1.58 mmol) and 1-(6-bromo-5-fluoro-1-methyl-indazol-3-yl)hexahydropyrimidine-2,4-dione (5, 537.95 mg, 1.58 mmol) in anhydrous 1,4-dioxane (10 mL) was added Cs₂CO₃ (1.54 g, 4.73 mmol) at room temperature under nitrogen atmosphere and resulting mixture was degassed by bubbling nitrogen gas into the reaction mixture for 5 min. Subsequently, Pd-PEPPSI-IHept(Cl) (153.40 mg, 157.70 mol) was added and stirred at 100° C. for 16 hours. After completion of the reaction, the reaction mixture was filtered through a Celite pad and washed with THF (30 mL), filtrate was concentrated under reduced pressure to get crude residue which was purified by silica gel column chromatography (100-200 mesh) using a gradient of 0-20% methanol/dichloromethane to afford tert-butyl 2-[1-[3-(2,4-dioxohexahydropyrimidin-1-yl)-5-fluoro-1-methyl-indazol-6-yl]-4-hydroxy-4-piperidyl]acetate (6, 160 mg, 217.03 mol, 14% yield) as light yellow gummy liquid. UPLC-MS (ES+): m/z 476.2 [M+H]⁺.

### Step 4

Into a 50 mL single-neck round-bottom flask containing a well stirred solution of tert-butyl 2-[1-[3-(2,4-dioxohexahydropyrimidin-1-yl)-5-fluoro-1-methyl-indazol-6-yl]-4-hydroxy-4-piperidyl]acetate (6, 160 mg, 217.03 mol) in dichloromethane (15 mL) was added trifluoroacetic acid (7.84 mmol, 0.6 mL) at 0° C. under nitrogen atmosphere. The reaction mixture was stirred at room temperature for 2 hours. After completion of the reaction, the reaction mixture was concentrated under reduced pressure and co-distilled with dichloromethane (50 mL) to afford 2-[1-[3-(2,4-dioxohexahydropyrimidin-1-yl)-5-fluoro-1-methyl-indazol-6-yl]-4-hydroxy-4-piperidyl]acetic acid (B-25, 110 mg, 121.87 mol, 56% yield, TFA salt) as a brown liquid. UPLC-MS (ES+): m/z 420.1 [M+H]⁺.

### Example 3: Synthesis of Intermediates

### Synthesis C₁: Synthesis of (3R)-3-[6-[2-cyano-3-[[ethyl(methyl)sulfamoyl]amino]-6-fluoro-phenoxy]-4-oxo-quinazolin-3-yl]-8-[(4-hydroxy-4-piperidyl)methyl]-1-oxa-8-azaspiro[4.5]decane (C-1)

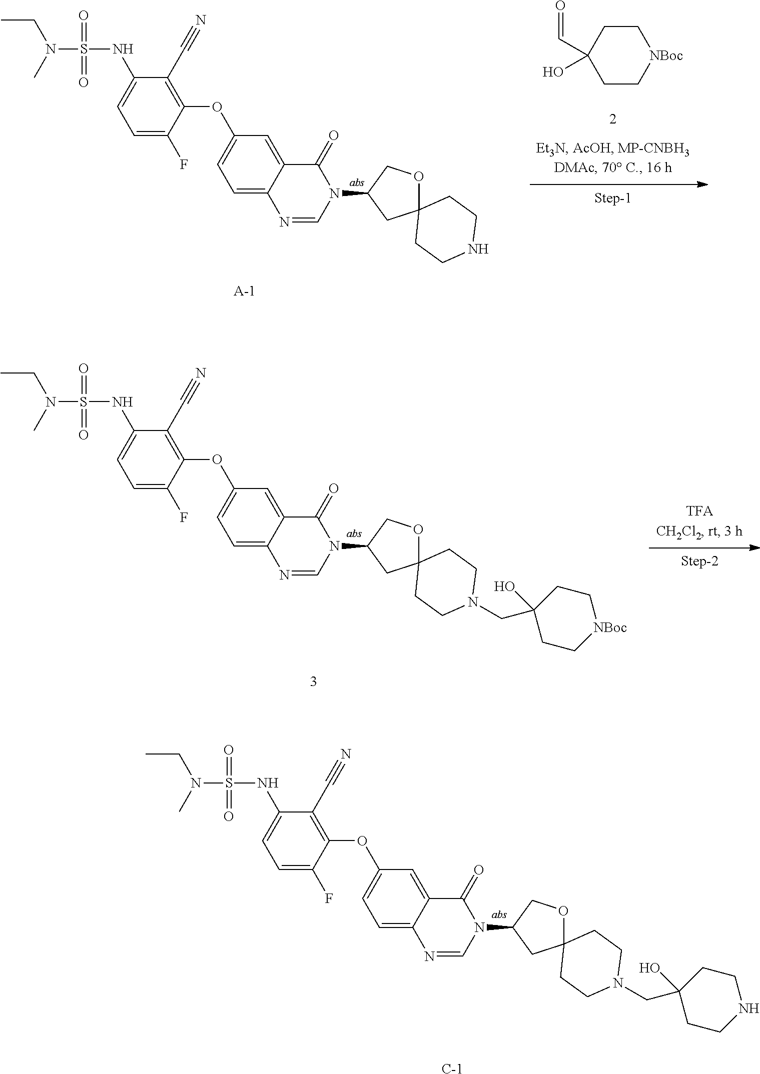

### Step 1

Into a 30 mL glass vial containing a well stirred solution of (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1) (500 mg, 745.57 mol, TFA salt) in DMAc (10 mL) was added triethylamine, 99% (2.24 mmol, 311.75 L) at 5° C. and stirred for 30 min. Subsequently, tert-butyl 4-formyl-4-hydroxy-piperidine-1-carboxylate (2, 307.69 mg, 1.34 mmol) and acetic acid (1.49 mmol, 85.27 μL) were added at room temperature under nitrogen atmosphere. The reaction mixture was stirred at room temperature for 3 hours. MP-CNBH₃ (600 mg, 745.57 mol) was added at room temperature and the mixture was stirred at 70° C. for 16 hours. After completion of the reaction, the reaction mixture was filtered and washed with ethyl acetate (20 mL). The filtrate was diluted with water (50 mL) and extracted with ethyl acetate (3×50 mL). The combined organic layer was washed with ice cold water (3×50 mL), saturated NaCl (50 mL) and dried over anhydrous Na₂SO₄ and concentrated under reduced pressure to get a crude residue which was purified by column chromatography (25 g, 230-400 silica-gel mesh) and eluted with 0-15% methanol in dichloromethane, while pure compound was eluted at 5% methanol in dichloromethane to afford tert-butyl 4-[[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]methyl]-4-hydroxy-piperidine-1-carboxylate (3, 290 mg, 302.66 mol, 41% yield) as an off-white solid. UPLC-MS (ES+): m/z 770.4 [M+H]⁺.

### Step 2

Into a 25 mL single neck round bottom flask containing a well stirred solution of tert-butyl 4-[[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]methyl]-4-hydroxy-piperidine-1-carboxylate (3, 290 mg, 376.68 mol) in dichloromethane (10 mL) was added trifluoroacetic acid (1.13 mmol, 86.48 μL) at 0° C. under nitrogen atmosphere. The reaction mixture was stirred at room temperature for 3 hours. After completion of the reaction, solvent was concentrated under reduced pressure to get the crude, which was neutralized with Amberlyst resin to afford (3R)-3-[6-[2-cyano-3-[[ethyl(methyl)sulfamoyl]amino]-6-fluoro-phenoxy]-4-oxo-quinazolin-3-yl]-8-[(4-hydroxy-4-piperidyl)methyl]-1-oxa-8-azaspiro[4.5]decane (C-1, 190 mg, 217.61 mol, 58%) as an off-white solid. UPLC-MS (ES+): m/z 670.2 [M+H]⁺.

### Synthesis C₂: Synthesis of 3-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-1-oxa-8-azaspiro[4.5]decane (C-2)

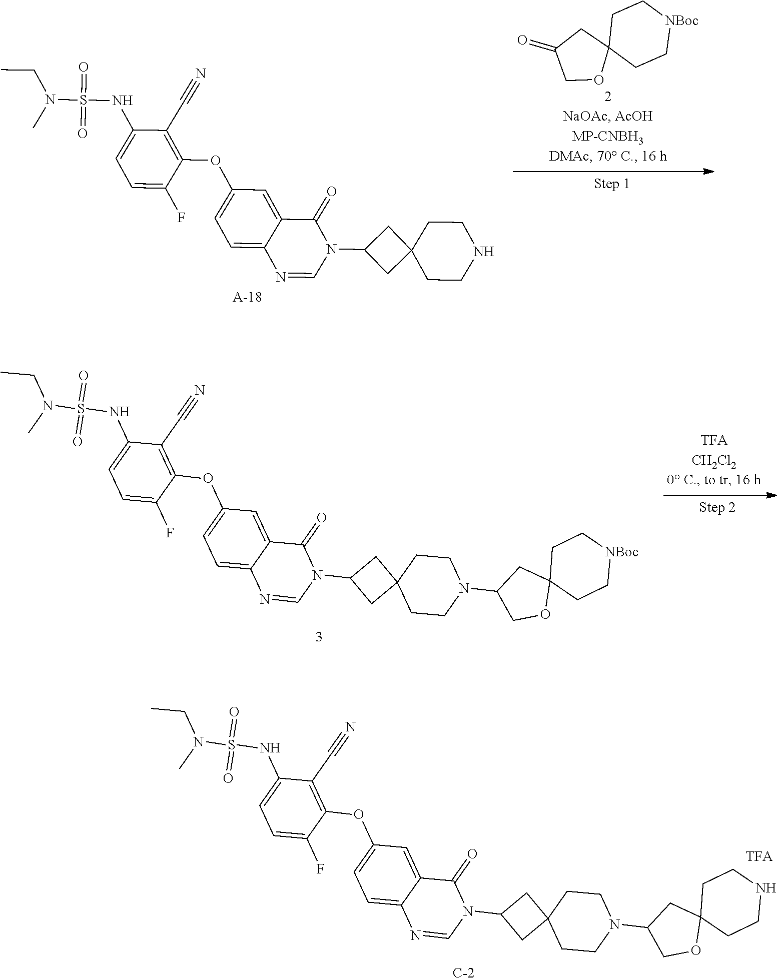

### Step 1

Into a 40 mL pressure vial containing a well-stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (1 g, 1.45 mmol, TFA salt) in DMAc (10 mL) was added anhydrous NaOAc (595.21 mg, 7.26 mmol) at room temperature under nitrogen atmosphere and mixture was stirred for 20 min. Reaction mixture was cooled to 0° C. and was added tert-butyl 3-oxo-1-oxa-8-azaspiro[4.5]decane-8-carboxylate (2, 555.76 mg, 2.18 mmol) followed by acetic acid (4.35 mmol, 248.98 L) and resulting mixture was stirred for 4 h at room temperature. MP-CNBH₃ (1 g, 1.45 mmol) was added, and the reaction mixture was stirred at 70° C. for 16 hours. After completion of the reaction, the reaction mixture was filtered, and filtrate was concentrated under reduced pressure to get a crude residue. Crude material was purified by flash silica gel (230-400 mesh) column chromatography by eluting with 0-70% ethyl acetate in pet-ether to afford tert-butyl 3-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-1-oxa-8-azaspiro[4.5]decane-8-carboxylate (3, 500 mg, 591.60 mol, 41% yield) as a brown solid. UPLC-MS (ES+): m/z 780.2 [M+H]⁺.

### Step 2

Into a 50 mL single-neck round-bottom flask containing a well-stirred solution of tert-butyl 3-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-1-oxa-8-azaspiro[4.5]decane-8-carboxylate (3, 500 mg, 641.09 mol) in anhydrous dichloromethane (10 mL) was added trifluoroacetic acid (3.21 mmol, 245.29 L) slowly at 0° C. under nitrogen atmosphere. The resulting solution was stirred at room temperature for 16 hours. After completion of the reaction, the reaction mixture was concentrated under reduced pressure and the obtained residue was washed with methyl tert-butyl ether to remove the non-polar impurity to afford 3-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-1-oxa-8-azaspiro[4.5]decane (C-2, 300 mg, 336.35 mol, 52% yield, TFA salt) as a brown gummy liquid. UPLC-MS (ES+): m/z 680.2 [M+H]⁺.

### Synthesis C₃: Synthesis of 6-[2-cyano-3-[[ethyl(methyl)sulfamoyl]amino]-6-fluoro-phenoxy]-3-[7-[(4-hydroxy-4-piperidyl)methyl]-7-azaspiro[3.5]nonan-2-yl]-4-oxo-quinazoline (C-3)

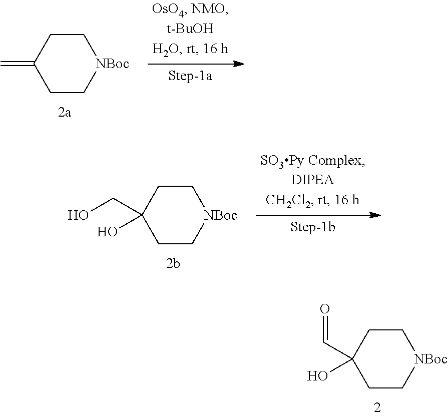 [SMILES: C=C1CCN(C(=O)OC(C)(C)C)CC1 ; CC(C)(C)OC(=O)N1CCC(O)(CO)CC1 ; CC(C)(C)OC(=O)N1CCC(O)(C=O)CC1]

### Step 1a

Into a 500 mL single neck round bottom flask containing a well stirred solution of tert-butyl 4-methylenepiperidine-1-carboxylate (2a, 3 g, 15.21 mmol) in t-BuOH (60 mL) and water (15 mL) were added osmium tetraoxide 4% in water (152.07 mol, 7.88 μL) followed by N-Methylmorpholine N-oxide (5.34 g, 45.62 mmol) at 0° C. The reaction mixture was stirred at room temperature for 16 hours. The reaction mixture was quenched with sodium sulfite (6 g) and stirred for 10 min. The reaction mixture was partially concentrated under reduced pressure, which was diluted with water (200 mL) and extracted with ethyl acetate (2×100 mL). The combined organic layer was dried over anhydrous Na₂SO₄, filtered and concentrated under reduced pressure to afford tert-butyl 4-hydroxy-4-(hydroxymethyl)piperidine-1-carboxylate (2b, 2.2 g, 9.30 mmol, 61% yield) as an off-white solid. UPLC-MS (ES+): m/z 132.2 [M−Boc+H]⁺.

### Step 1b

Into a 50 mL single neck round bottom flask containing a well stirred solution of tert-butyl 4-hydroxy-4-(hydroxymethyl)piperidine-1-carboxylate (2b, 1 g, 4.32 mmol) in dichloromethane (10 mL) and DMSO (3 mL) were added N,N-diisopropylethylamine (17.29 mmol, 3.01 mL) and pyridine sulfur trioxide (1.38 g, 8.65 mmol) at 0° C. under nitrogen atmosphere. The resulting reaction mixture was stirred at room temperature for 16 hours. The reaction mixture was diluted with dichloromethane (30 mL) and washed with water (2×50 mL), 0.1 M HCl solution (30 mL). The organic layer was dried over anhydrous Na₂SO₄ and concentrated under reduced pressure to get the crude, which was washed with hexane (20 mL) and dried under reduced pressure to afford tert-butyl 4-formyl-4-hydroxy-piperidine-1-carboxylate (2, 320 mg, 347.12 mol, 8% yield) as a colorless liquid. The crude was taken to the next step without purification. ELSD-MS (ES+): m/z 174.0 [M−56+H]⁺.

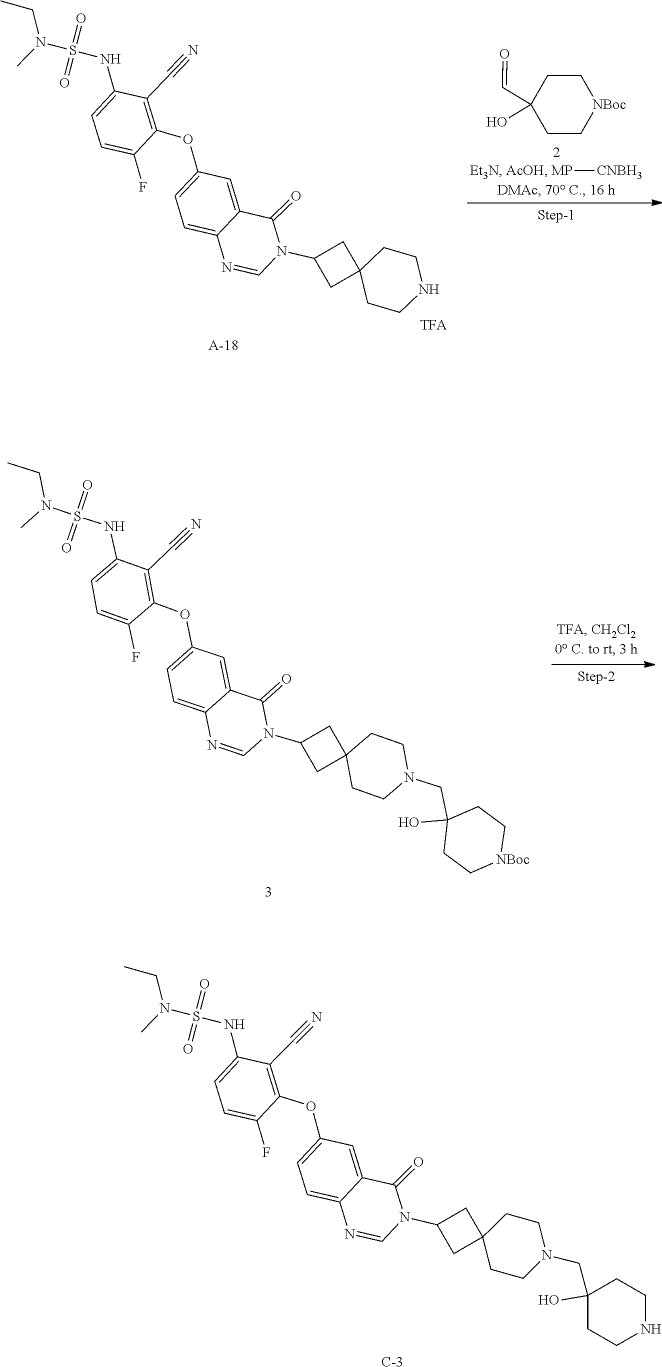

### Step 1

Into a 30 mL glass vial containing a well stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (300 mg, 458.27 mol, TFA salt) in DMAc (10 mL) was added triethylamine, 99% (1.37 mmol, 191.62 L) at 0° C. and stirred for 30 min. Subsequently, tert-butyl 4-formyl-4-hydroxy-piperidine-1-carboxylate (2, 189.12 mg, 824.89 mol) and acetic acid (916.55 mol, 52.41 μL) were added at room temperature. The reaction mixture was stirred at room temperature for 3 hours. MP-CNBH₃ (200 mg, 305.52 mol) was added at room temperature under nitrogen atmosphere. The resulting mixture was stirred at 70° C. for 16 hours. After completion of the reaction, the reaction mixture was filtered, and the filtrate was diluted with water (50 mL) and extracted with ethyl acetate (3×50 mL). The combined organic layer was washed with ice cold water (3×50 mL), saturated NaCl (50 mL) and dried over anhydrous sodium sulphate and concentrated under reduced pressure to get the crude compound, which was purified by flash column chromatography (230-400 silica-gel mesh, 25 g) and eluted with 0-15% methanol in dichloromethane, while pure compound was eluted at 5% methanol in dichloromethane to afford tert-butyl 4-[[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]methyl]-4-hydroxy-piperidine-1-carboxylate (3, 230 mg, 275.25 mol, 60% yield) as an off-white solid. UPLC-MS (ES+): m/z 754.3 [M+H]⁺.

### Step 2

Into a 25 mL single neck round bottom flask containing a well stirred solution of tert-butyl 4-[[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]methyl]-4-hydroxy-piperidine-1-carboxylate (3, 230 mg, 305.09 mol) in dichloromethane (10 mL) was added trifluoroacetic acid (1.53 mmol, 116.74 L) at 0° C. under nitrogen atmosphere. The reaction mixture was stirred at room temperature for 3 hours. After completion of the reaction, the reaction mixture was concentrated under reduced pressure to get the crude, which was neutralized with Amberlyst resin to afford 6-[2-cyano-3-[[ethyl(methyl)sulfamoyl]amino]-6-fluoro-phenoxy]-3-[7-[(4-hydroxy-4-piperidyl)methyl]-7-azaspiro[3.5]nonan-2-yl]-4-oxo-quinazoline (C-3, 150 mg, 203.03 mol, 67% yield) as a pale yellow gum. UPLC-MS (ES+): m/z 654.3 [M+H]⁺.

### Synthesis C₄: Synthesis of 6-[2-cyano-3-[[ethyl(methyl)sulfamoyl]amino]-6-fluoro-phenoxy]-3-[7-(3,3-difluoro-4-piperidyl)-7-azaspiro[3.5]nonan-2-yl]-4-oxo-quinazoline (C-4)

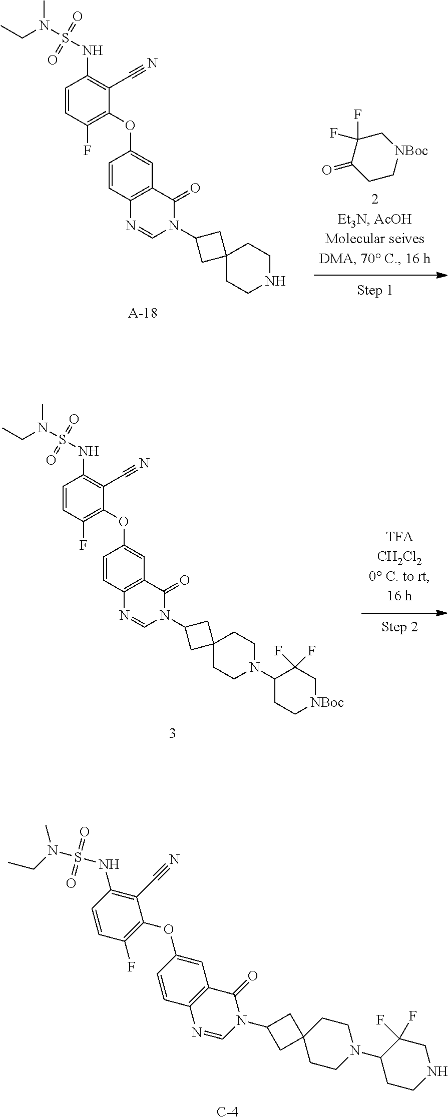

### Step 1

Into a 20 mL pressure vial containing a well stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (500 mg, 924.88 mol) dissolved in DMAc (10 mL) were added triethylamine (4.62 mmol, 644.55 L), molecular sieves 4 A° powder (400 mg, 924.88 mol) and stirred for 30 min. Subsequently, tert-butyl 3,3-difluoro-4-oxo-piperidine-1-carboxylate (2, 326.34 mg, 1.39 mmol) and acetic acid (924.88 mol, 52.89 μL) were added and stirred at 70° C. for 5 hours. MP-CNBH₃ (500 mg, 7.96 mmol) was added and stirred at 70° C. for 16 hours. The reaction mixture was filtered through cotton pad and filtrate was evaporated under reduced pressure to afford crude residue which was purified by reverse phase column chromatography (Column: Redisep R_{f} Gold® reversed-phase C₁₈-Teledyne ISCO, 120 g, Mobile phase A: 10 mm NH₄HCO₃ in milli Q-water; Mobile phase B: acetonitrile). Fractions were collected and lyophilized to afford tert-butyl4-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-3,3-difluoro-piperidine-1-carboxylate (3, 200 mg, 210.57 mol, 23% yield) as a brown gum. UPLC-MS (ES+): m/z 760.3 [M+H]⁺.

### Step 2

Into a 25 mL two-neck round-bottom flask containing a well-stirred solution of tert-butyl 4-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-3,3-difluoro-piperidine-1-carboxylate (3, 200 mg, 263.21 mol) in dichloromethane (5 mL) was added trifluoroacetic acid (2.63 mmol, 201.43 L) at 0° C. under nitrogen atmosphere. Resulting reaction mixture was stirred at room temperature for 16 hours. The reaction mixture was concentrated under reduced pressure to afford a crude. Crude was dissolved in 10% methanol/dichloromethane, neutralized with NaHCO₃ (pH was adjusted to 8) and filtered. Filtrate was concentrated under reduced pressure to afford 6-[2-cyano-3-[[ethyl(methyl)sulfamoyl]amino]-6-fluoro-phenoxy]-3-[7-(3,3-difluoro-4-piperidyl)-7-azaspiro[3.5]nonan-2-yl]-4-oxo-quinazoline (C-4, 160 mg, 155.09 mol, 59% yield) as an off-white solid. UPLC-MS (ES+): m/z 660.3 [M+H]⁺.

### Synthesis C5: Synthesis of 3-[7-(2-azaspiro[3.5]nonan-7-yl)-7-azaspiro[3.5]nonan-2-yl]-6-[2-cyano-3-[[ethyl(methyl)sulfamoyl]amino]-6-fluoro-phenoxy]-4-oxo-quinazoline (C-5)

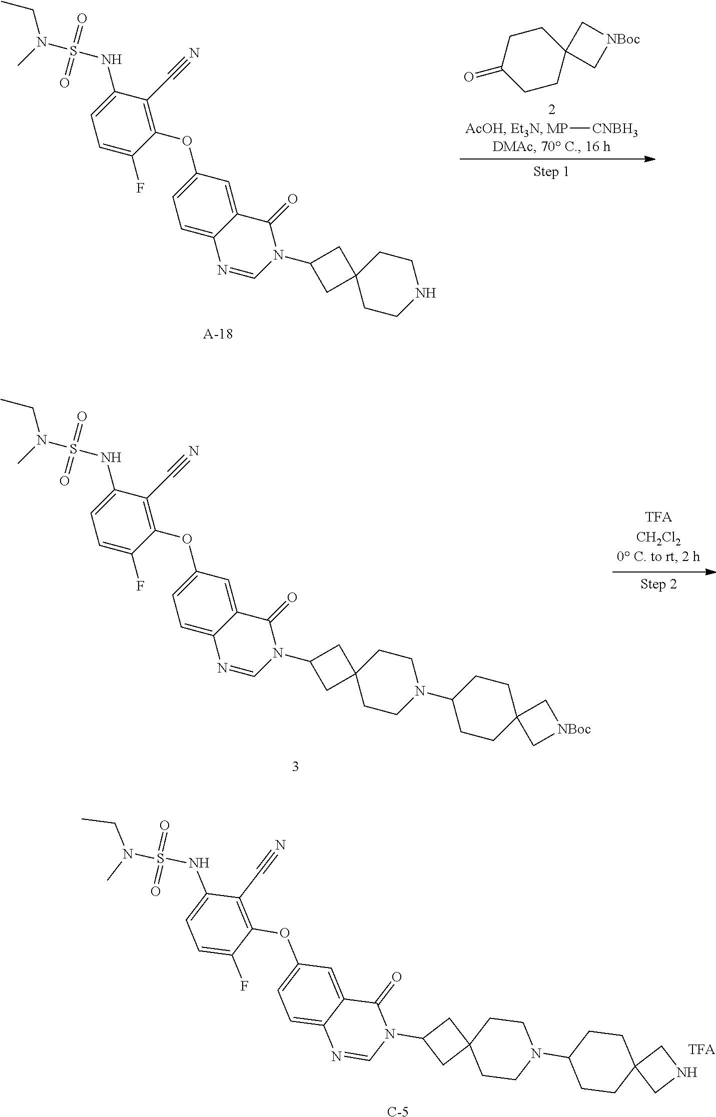

### Step 1

Into a 40 mL pressure vial containing a well-stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (800 mg, 1.48 mmol) in DMAc (20 mL) was added triethylamine (4.44 mmol, 618.77 L) at room temperature under nitrogen atmosphere and mixture was stirred for 20 min. Reaction mixture was cooled to 0° C. and was added tert-butyl 7-oxo-2-azaspiro[3.5]nonane-2-carboxylate (2, 354.13 mg, 1.48 mmol) followed by AcOH (7.40 mmol, 423.15 μL). The resulting mixture was stirred for 3 h at room temperature. MP-CNBH₃ (800 mg, 1.48 mmol) was added, and the reaction mixture was stirred at 70° C. for 16 hours. The reaction mixture was filtered and the filtrate was concentrated under reduced pressure to get a crude residue which was purified by reverse phase column chromatography (Redisep R_{f} Gold®reversed-phase C18-teledyne ISCO, 120 g), [Mobile phase A: 10 mm NH₄HCO₃ in milli-Q water; Mobile phase B: acetonitrile]. The fractions were lyophilized to afford tert-butyl 7-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-2-azaspiro[3.5]nonane-2-carboxylate (3, 385 mg, 469.10 mol, 32% yield) as a white solid. UPLC-MS (ES+): m/z 764.5 [M+H]⁺.

### Step 2

Into a 50 mL single-neck round-bottom flask containing a well-stirred solution of tert-butyl 7-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-2-azaspiro[3.5]nonane-2-carboxylate (3, 385 mg, 503.98 mol) in dichloromethane (10 mL) was added trifluoroacetic acid (5.04 mmol, 385.67 L) slowly at 0° C. under nitrogen atmosphere. The resulting solution was stirred at room temperature for 2 hours. The reaction mixture was concentrated under reduced pressure and the obtained residue was washed with methyl tert-butyl ether to remove the non-polar impurity to afford 3-[7-(2-azaspiro[3.5]nonan-7-yl)-7-azaspiro[3.5]nonan-2-yl]-6-[2-cyano-3-[[ethyl(methyl)sulfamoyl]amino]-6-fluoro-phenoxy]-4-oxo-quinazoline (C-5), 390 mg, 434.36 mol, 86% yield, TFA salt) as a brown gum. UPLC-MS (ES+): m/z 664.3 [M+H]⁺.

### Example 4: Synthesis of Degraders

### Synthesis D2: Synthesis of (3R)-3-[6-[2-cyano-3-[[ethyl(methyl)sulfamoyl]amino]-6-fluoro-phenoxy]-4-oxo-quinazolin-3-yl]-8-[4-[4-[[(3S)-2,6-dioxo-3-piperidyl]amino]-2-fluoro-phenyl]cyclohexyl]-1-oxa-8-azaspiro[4.5]decane (Compound 3 and Compound 4)

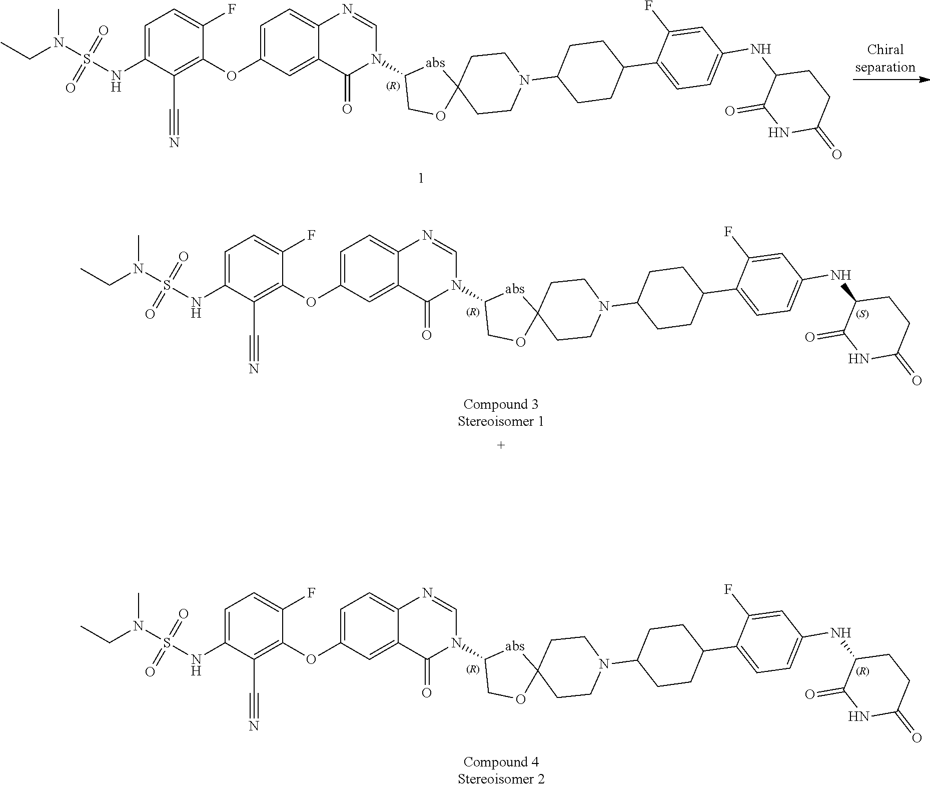

Compound (3R)-3-[6-[2-cyano-3-[[ethyl(methyl)sulfamoyl]amino]-6-fluoro-phenoxy]-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (1, 80.00 mg, 90.04 mol) was purified by chiral SFC following the below method.

### Method of Analysis for chiral SFC separation

Instrument: PIC-22-016
Column: Chiral Pak-AS-H (250*20) mm, 5 m
Mobile Phase: CO₂: 0.5% IPAm in MeCN: methanol [60:40]
Total Flow: 60 mL/min
Back pressure: 100 bar
Wavelength: 251 nm
Cycle time: 7 min

Fast eluting isomer (Peak-1): Fast eluting compound (Peak-1, t_{R}=1.03 min) was collected and concentrated under reduced pressure to afford (3R)-3-[6-[2-cyano-3-[[ethyl(methyl)sulfamoyl]amino]-6-fluoro-phenoxy]-4-oxo-quinazolin-3-yl]-8-[4-[4-[[(3S)-2,6-dioxo-3-piperidyl]amino]-2-fluoro-phenyl]cyclohexyl]-1-oxa-8-azaspiro[4.5]decane (30 mg, 34.74 mol, 39% yield) as an off-white solid. Peak-1 was assigned as stereoisomer 1. UPLC-MS (ES+): m/z 859.2 [M+H]⁺. ¹H NMR (400 MHz, DMSO-d₆): δ 10.79 (s, 1H), 8.55 (bs, 1H), 8.32 (s, 1H), 7.76 (d, J=8.80 Hz, 1H), 7.64 (dd, J=2.80, 8.80 Hz, 1H), 7.15-7.55 (m, 3H), 7.07 (s, 1H), 6.49-6.43 (m, 2H), 6.01 (d, J=7.20 Hz, 1H), 5.32-5.29 (m, 1H), 4.35-4.27 (m, 1H), 4.21-4.08 (m, 2H), 3.68-3.42 (m, 1H), 3.10-2.80 (m, 6H), 2.80-2.68 (m, 1H), 2.60-2.62 (m, 4H), 2.32-2.43 (m, 3H), 2.04-2.13 (m, 2H), 1.75-2.03 (m, 10H), 1.53-1.70 (m, 2H), 1.03 (t, J=7.20 Hz, 3H).

Late eluting isomer (Peak-2): Late eluting compound (Peak-2, t_{R}=1.49 min) was collected and concentrated under reduced pressure to afford (3R)-3-[6-[2-cyano-3-[[ethyl(methyl)sulfamoyl]amino]-6-fluoro-phenoxy]-4-oxo-quinazolin-3-yl]-8-[4-[4-[[(3R)-2,6-dioxo-3-piperidyl]amino]-2-fluoro-phenyl]cyclohexyl]-1-oxa-8-azaspiro[4.5]decane (28 mg, 32.20 mol, 36% yield) as an off-white solid. Peak-2 was assigned as stereoisomer 2. UPLC-MS (ES+): m/z 859.2 [M+H]+. ¹H NMR (400 MHz, DMSO-d₆): δ 10.79 (s, 1H), 8.63 (bs, 1H), 8.31 (s, 1H), 7.76 (d, J=8.80 Hz, 1H), 7.64 (dd, J=3.20, 9.00 Hz, 1H), 7.50-7.23 (m, 3H), 7.07 (s, 1H), 6.49-6.43 (m, 2H), 6.01 (d, J=7.20 Hz, 1H), 5.33-5.25 (m, 1H), 4.33-4.27 (m, 1H), 4.21-4.08 (m, 2H), 3.2-0-2.80 (m, 6H), 2.79-2.66 (m, 2H), 2.63-2.58 (m, 4H), 2.43-2.35 (m, 2H), 2.13-2.03 (m, 2H), 2.04-1.70 (m, 10H), 1.66-1.52 (m, 2H), 1.19-1.35 (m, 1H), 1.03 (t, J=7.20 Hz, 3H).

### Synthesis D4: Synthesis of 6-[2-cyano-3-[[ethyl(methyl)sulfamoyl]amino]-6-fluoro-phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 6 and Compound 7)

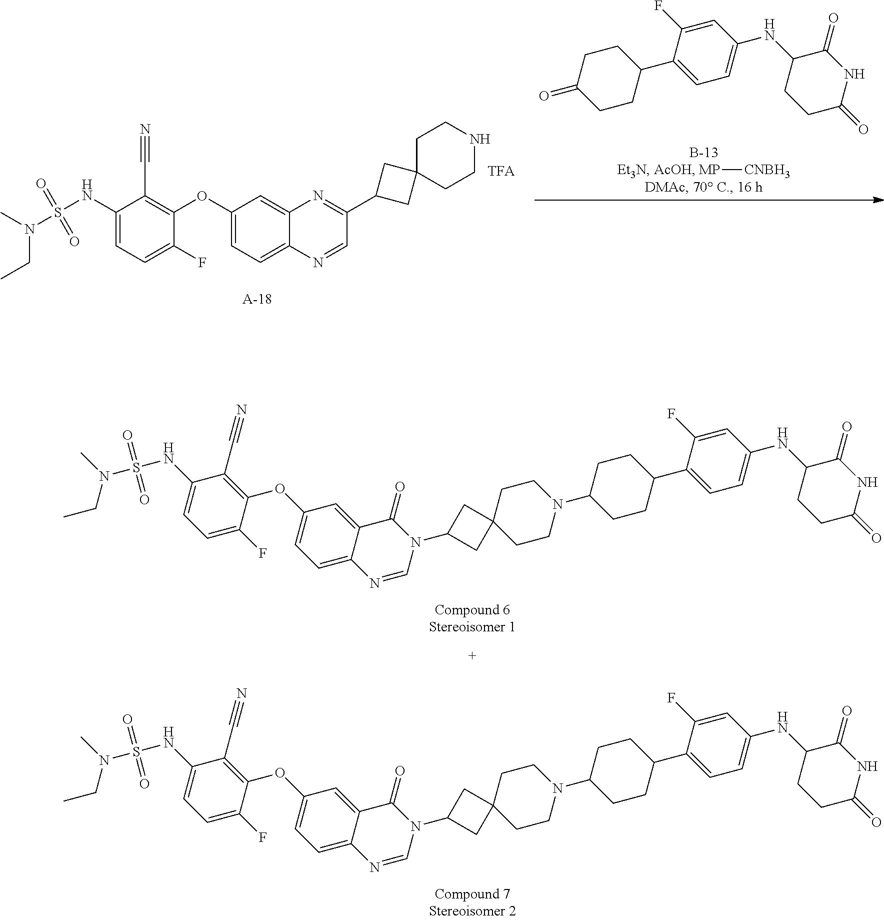

Into a 30 mL pressure vial containing a well stirred solution of 3-(7-azaspiro[3.5]nonan-2-yl)-6-[2-cyano-3-[[ethyl(menthyl)sulfamoyl]amino]-6-fluoro-phenoxy]-4-oxo-quinazoline (A-18) (0.2 g, 305.52 μmol, TFA salt) in DMAc (12 mL) was added triethylamine (1.53 mmol, 212.91 μL) at room temperature under nitrogen atmosphere and mixture was stirred for 30 minutes. Reaction mixture was cooled to 0° C. and was added [3-fluoro-4-(4-oxocyclohexyl)anilino]piperidine-2,6-dione (B-13) (194.52 mg, 611.03 μmol) followed by AcOH (916.55 μmol, 52.47 μL) and resulting mixture was stirred for 3 hours at room temperature. MP-CNBH₃ (0.400 g, 305.52 μmol) was added, and the reaction mixture was stirred at 70° C. for 16 hours. After completion of the reaction, the reaction mixture was filtered, and filtrate was concentrated under reduced pressure to get a crude residue which was purified by reverse phase column chromatography (Redisep RfGold®reversed-phase C18-teledyne ISCO, 120 g), [Mobile phase A: 10 mM NH₄HCO₃ in milli-Q water; Mobile phase B: acetonitrile]. The fractions were lyophilized to afford 0.2 g of mixture of Stereoisomer 1 and Stereoisomer 2. Above isomeric mixture of 6-[2-cyano-3-[[ethyl(methyl)sulfamoyl]amino]-6-fluoro-phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (0.200 g, 218.28 μmol) was purified by chiral SFC following the below method.

### Method of Analysis for Chiral SFC Separation

Instrument: PIC-175
Column: Diol(250*30)mm, 5 m
Mobile Phase: CO₂: 0.5% IPAm in IPA [60:40]
Total Flow: 120 mL/min
Back pressure: 120 bar
Wavelength: 220 nm
Cycle time: 16.0 min

Fast eluting isomer (Peak-1): Fast eluting compound (Peak-1, t_{R}=2.07 min) was collected and concentrated under reduced pressure to afford 6-[2-cyano-3-[[ethyl(methyl)sulfamoyl]amino]-6-fluoro-phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 6, 35 mg, 39.93 μmol, 18% yield) as an off-white solid. Peak-1 was assigned as stereoisomer 1. UPLC-MS (ES+): m/z 843.2 [M+H]+. ¹H NMR (400 MHz, DMSO-d₆): δ 10.78 (s, 1H), 8.37 (s, 1H), 7.74 (d, J=8.80 Hz, 1H), 7.59 (dd, J=3.20, 8.80 Hz, 1H), 7.33-7.19 (m, 3H), 7.00 (t, J=8.80 Hz, 1H), 6.48-6.39 (m, 2H), 5.98 (d, J=7.60 Hz, 1H), 5.02-4.82 (m, 1H), 4.38-4.22 (m, 1H), 2.97 (q, J=7.20 Hz, 2H), 2.84-2.63 (m, 3H), 2.63-2.57 (m, 1H), 2.54 (s, 3H), 2.39-2.31 (m, 2H), 2.30-2.22 (m, 2H), 2.19-2.05 (m, 1H), 2.04-1.94 (m, 2H), 1.92-1.63 (m, 7H), 1.62-1.33 (m, 4H), 1.02 (t, J=7.20 Hz, 3H).

Late eluting isomer (Peak-2): Late eluting compound (Peak-2, t_{R}=3.40 min) was collected and concentrated under reduced pressure to afford 6-[2-cyano-3-[[ethyl(methyl)sulfamoyl]amino]-6-fluoro-phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 7, 29 mg, 33.31 μmol, 15% yield) as an off-white solid. Peak-2 was assigned as stereoisomer 2.UPLC-MS (ES+): m/z 843.2 [M+H]+. ¹H NMR (400 MHz, DMSO-d₆): δ 10.82 (s, 1H), 8.36 (s, 1H), 7.73 (d, J=9.20 Hz, 1H), 7.58 (dd, J=3.20, 9.2 Hz, 1H), 7.36-7.18 (m, 3H), 6.97 (t, J=8.80 Hz, 1H), 6.48-6.38 (m, 2H), 5.96 (d, J=8.00 Hz, 1H), 4.99-4.83 (m, 1H), 4.37-4.23 (m, 1H), 2.96 (q, J=7.20 Hz, 2H), 2.80-2.69 (m, 1H), 2.62-2.57 (m, 1H), 2.45-2.39 (m, 2H), 2.33-2.27 (m, 2H), 2.25-2.16 (m, 2H), 2.13-2.01 (m, 1H), 1.91-1.72 (m, 6H), 1.71-1.54 (m, 4H), 1.53-1.29 (m, 4H), 1.02 (t, J=7.20 Hz, 3H).

### Synthesis D5: Synthesis of 6-[2-cyano-3-[[ethyl(methyl)sulfamoyl]amino]-6-fluoro-phenoxy]-3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazoline (Compound 8 and Compound 9)

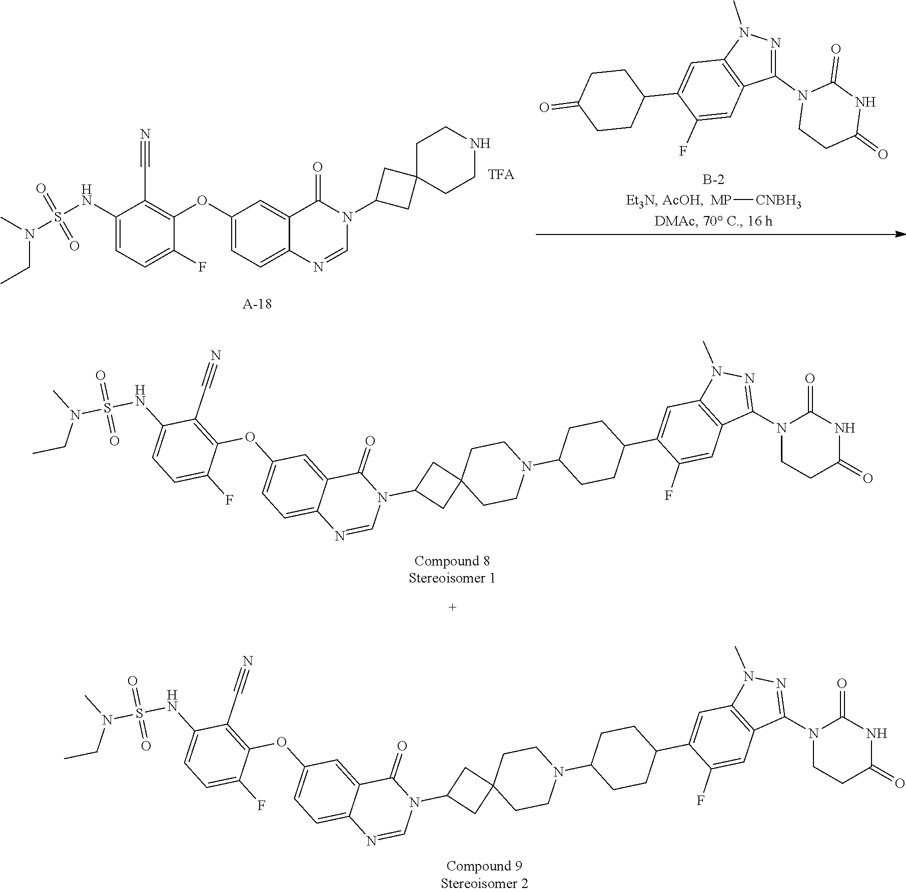

Into a 30 mL pressure vial containing a well stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (800 mg, 1.22 mmol, TFA salt) in DMAc (12 mL) was added triethylamine (6.11 mmol, 851.66 μL) at room temperature under nitrogen atmosphere and mixture was stirred for 30 minutes. Reaction mixture was cooled to 0° C. and was added 1-[5-fluoro-1-methyl-6-(4-oxocyclohexyl)indazol-3-yl]hexahydropyrimidine-2,4-dione (B-2) (656.92 mg, 1.83 mmol) followed by AcOH (3.67 mmol, 209.87 μL) and resulting mixture was stirred for 3 hours at room temperature. MP-CNBH₃ (800 mg, 1.53 mmol) was added, and the reaction mixture was stirred at 70° C. for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fractions were lyophilized to afford 1 g of mixture of stereoisomer 1 and stereoisomer 2. Further stereoisomer 1 and stereoisomer 2 were separated by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction (Peak-1) was concentrated under reduced pressure at 40° C. and lyophilized to afford Compound 8. Peak-1 was assigned as stereoisomer 1. Peak-1: 6-[2-cyano-3-[[ethyl(methyl)sulfamoyl]amino]-6-fluoro-phenoxy]-3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazoline (Compound 8, 317 mg, 352.41 mol, 30% yield) as an off-white solid. UPLC-MS (ES+): m/z 883.4 [M+H]+. ¹H NMR (400 MHz, DMSO-d₆): δ 10.55 (s, 1H), 8.36 (s, 1H), 7.75 (d, J=8.80 Hz, 1H), 7.62 (dd, J=3.20, 9.20 Hz, 1H), 7.51 (d, J=6.0 Hz, 1H), 7.48-7.34 (m, 2H), 7.33-7.24 (m, 2H), 5.02-4.88 (m, 1H), 3.99 (s, 3H), 3.89 (t, J=6.80 Hz, 3H), 3.23-3.19 (m, 2H), 3.00 (q, J=7.20 Hz, 2H), 2.94-2.81 (m, 3H), 2.75 (t, J=6.80 Hz, 3H), 2.59 (s, 3H), 2.47-2.32 (m, 3H), 2.23-2.12 (m, 2H), 2.10-1.81 (m, 6H), 1.75-1.58 (m, 4H), 1.02 (t, J=7.20 Hz, 3H).

Similarly, the second eluted fraction (Peak-2) was concentrated under reduced pressure at 40° C. and lyophilized to afford Compound 9 (assigned as stereoisomer 2., t_{R}=6.24 mentioned based on SFC). Peak-2: 6-[2-cyano-3-[[ethyl(methyl)sulfamoyl]amino]-6-fluoro-phenoxy]-3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazoline (Compound 9, 420 mg, 461.63 mol, 40% yield) as an off-white solid. UPLC-MS (ES+): m/z 883.3 [M+H]+. ¹H NMR (400 MHz, DMSO-d₆): δ 10.56 (s, 1H), 8.38 (s, 1H), 7.76 (d, J=9.20 Hz, 1H), 7.62 (q, J=3.20, 8.80 Hz, 1H), 7.48 (d, J=6.00 Hz, 1H), 7.43-7.37 (m, 1H), 7.36-7.32 (m, 1H), 7.31-7.23 (m, 1H), 5.08-4.87 (m, 1H), 4.03 (s, 3H), 3.91 (t, J=6.80 Hz, 2H), 3.23-3.19 (m, 2H), 3.08-2.93 (m, 3H), 2.76 (t, J=6.80 Hz, 3H), 2.64-2.58 (m, 3H), 2.42-2.28 (m, 4H), 2.13-1.59 (m, 13H), 1.03 (t, J=7.20 Hz, 3H).

### Synthesis D6: Synthesis of 9-[6-[2-cyano-3-[[ethyl(methyl)sulfamoyl]amino]-6-fluoro-phenoxy]-4-oxo-quinazolin-3-yl]-3-[4-[4-[(2,6-dioxo-3-piperidyl)amino]-2-fluoro-phenyl]cyclohexyl]-3-azaspiro[5.5]undecane (Compound 10 and Compound 11)

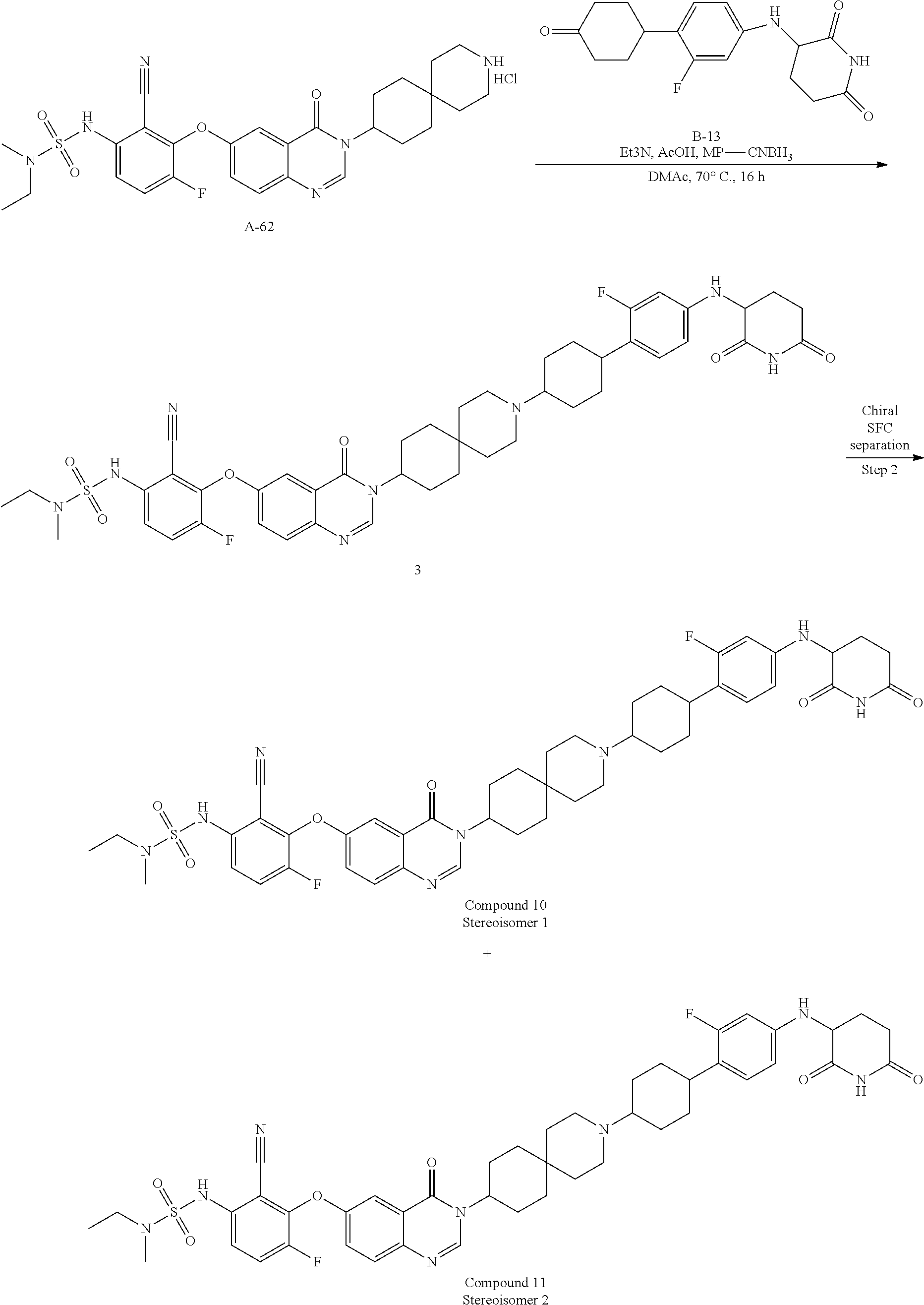

Into a 20 mL pressure vial containing a well stirred solution of 9-[6-[2-cyano-5-[[ethyl(methyl)sulfamoyl]amino]-6-fluoro-phenoxy]-4-oxo-quinazolin-3-yl]-3-azaspiro[5.5]undecane (A-62) (300 mg, 426.26 mol, HCl salt) and 3-[3-fluoro-4-(4-oxocyclohexyl)anilino]piperidine-2,6-dione (B-13) (291.82 mg, 852.53 mol) in DMAc (6 mL) was added triethylamine (1.28 mmol, 178.24 μL) at room temperature and the resulting mixture was stirred for 30 minutes at the same temperature. After 1 hour, AcOH (426.26 mol, 24.40 μL) was added at 0° C. and stirring was continued at room temperature for 1 hour. Subsequently, MP-CNBH₃ (600 mg) was added, and resulting reaction mixture was stirred at 70° C. for 16 hours. After the completion of the reaction, the reaction mixture was filtered through cotton pad and the filtrate was concentrated under reduced pressure to get a crude residue which was purified by reverse phase column chromatography (Column: Redisep Rf Gold® reversed-phase C18-teledyne ISCO, 120 g), [Mobile phase A: 10 mM ammonium bicarbonate in milli-Q water; Mobile phase B: acetonitrile]. Fractions were collected and lyophilized to afford a isomeric mixture of 9-[6-[2-cyano-3-[[ethyl(methyl)sulfamoyl]amino]-6-fluoro-phenoxy]-4-oxo-quinazolin-3-yl]-3-[4-[4-[(2,6-dioxo-3-piperidyl)amino]-2-fluoro-phenyl]cyclohexyl]-3-azaspiro[5.5]undecane (3, 200 mg, 218.12 mol, 51% yield) as an off-white solid. UPLC-MS (ES+): m/z 871.2 [M+H]+.

Step 2: Chiral SFC separation: Above isomeric mixture of 9-[6-[2-cyano-3-[[ethyl(methyl)sulfamoyl]amino]-6-fluoro-phenoxy]-4-oxo-quinazolin-3-yl]-3-[4-[4-[(2,6-dioxo-3-piperidyl)amino]-2-fluoro-phenyl]cyclohexyl]-3-azaspiro[5.5]undecane (3, 200 mg, 217.45 mol) was purified by chiral SFC following the below method.

### Method of Analysis for Chiral SFC Separation

Instrument: PIC 175
Column: 4 EP-(250*30)mm, 5 m
Mobile Phase: CO₂: 0.5% IPAm in IPA[60:40]
Total Flow: 120 mL/min
Back pressure: 120 bar
Wavelength: 220 nm
Cycle time: 9.5 min

Fast eluting isomer (Peak-1): Fast eluting compound (Peak-1, t_{R}=5.403 min) was collected and concentrated under reduced pressure to afford 9-[6-[2-cyano-3-[[ethyl(methyl)sulfamoyl]amino]-6-fluoro-phenoxy]-4-oxo-quinazolin-3-yl]-3-[4-[4-[(2,6-dioxo-3-piperidyl)amino]-2-fluoro-phenyl]cyclohexyl]-3-azaspiro[5.5]undecane (Compound 10, 35.16 mg, 37.97 mol, 17% yield) as an off-white solid. Peak-1 was assigned as stereoisomer 1. UPLC-MS (ES+): m/z 871.3 [M+H]+. ¹H NMR (400 MHz, DMSO-d₆): δ 10.79 (s, 1H), 8.49 (s, 1H), 8.38 (bs, 1H), 7.74 (d, J=9.20 Hz, 1H), 7.62 (dd, J=2.80, 8.80 Hz, 1H), 7.45-7.32 (m, 2H), 7.26 (bs, 1H), 7.18-7.03 (m, 1H), 6.51-6.44 (m, 2H), 6.03 (d, J=4.80 Hz, 1H), 4.59-4.48 (m, 1H), 4.35-4.25 (m, 1H), 3.51-3.40 (m, 1H), 3.15-2.88 (m, 4H), 2.80-2.69 (m, 1H), 2.63-2.54 (m, 4H), 2.30-2.18 (m, 1H), 2.03-1.79 (m, 10H), 1.75-1.35 (m, 10H), 1.28-1.10 (m, 2H), 1.02 (t, J=7.20 Hz, 3H).

Late eluting isomer (Peak-2): Late eluting compound (Peak-2, t_{R}=6.87 min) was collected and concentrated under reduced pressure to afford 9-[6-[2-cyano-3-[[ethyl(methyl)sulfamoyl]amino]-6-fluoro-phenoxy]-4-oxo-quinazolin-3-yl]-3-[4-[4-[(2,6-dioxo-3-piperidyl)amino]-2-fluoro-phenyl]cyclohexyl]-3-azaspiro[5.5]undecane which was not having desired purity (Chiral purity 85.45%). So the compound was further purified by PREP HPLC (Column: X-BRIDGE C8 (150 mm*19 mm*5 μm), Mobile phase A: 10 mm ammonium Bicarbonate in water, Mobile phase B: acetonitrile, flow rate: 15 ml/min, t_{R}:11.07). Fractions were collected and lyophilized to afford 9-[6-[2-cyano-3-[[ethyl(methyl)sulfamoyl]amino]-6-fluoro-phenoxy]-4-oxo-quinazolin-3-yl]-3-[4-[4-[(2,6-dioxo-3-piperidyl)amino]-2-fluoro-phenyl]cyclohexyl]-3-azaspiro[5.5]undecane (Compound 11, 6.5 mg, 7.34 mol, 3% yield) as a white solid. Peak-2 was assigned as stereoisomer 2. UPLC-MS (ES+): m/z 871.3 [M+H]+. ¹H NMR (400 MHz, DMSO-d₆): δ 10.79 (s, 1H), 8.82 (bs, 1H), 8.49 (s, 1H), 7.74 (d, J=8.80 Hz, 1H), 7.61 (dd, J=3.20, 9.00 Hz, 1H), 7.34-7.20 (m, 3H), 6.98 (t, J=8.40 Hz, 1H), 6.47-6.42 (m, 2H), 6.02 (d, J=7.60 Hz, 1H), 4.60-4.48 (m, 1H), 4.35-4.27 (m, 1H), 3.15-2.95 (m, 4H), 2.80-2.69 (m, 1H), 2.66-2.54 (m, 5H), 2.28-1.95 (m, 6H), 1.92-1.80 (m, 3H), 1.75-1.38 (m, 11H), 1.30-1.10 (m, 1H), 1.02 (t, J=7.20 Hz, 3H).

### Synthesis D7: Synthesis of (9-[6-[2-cyano-3-[[ethyl(methyl)sulfamoyl]amino]-6-fluoro-phenoxy]-4-oxo-quinazolin-3-yl]-3-[4-[3-(2,4-dioxohexahydropyrimidin-1-yl)-5-fluoro-1-methyl-indazol-6-yl]cyclohexyl]-3-azaspiro[5.5]undecane (Compound 12 and Compound 13)

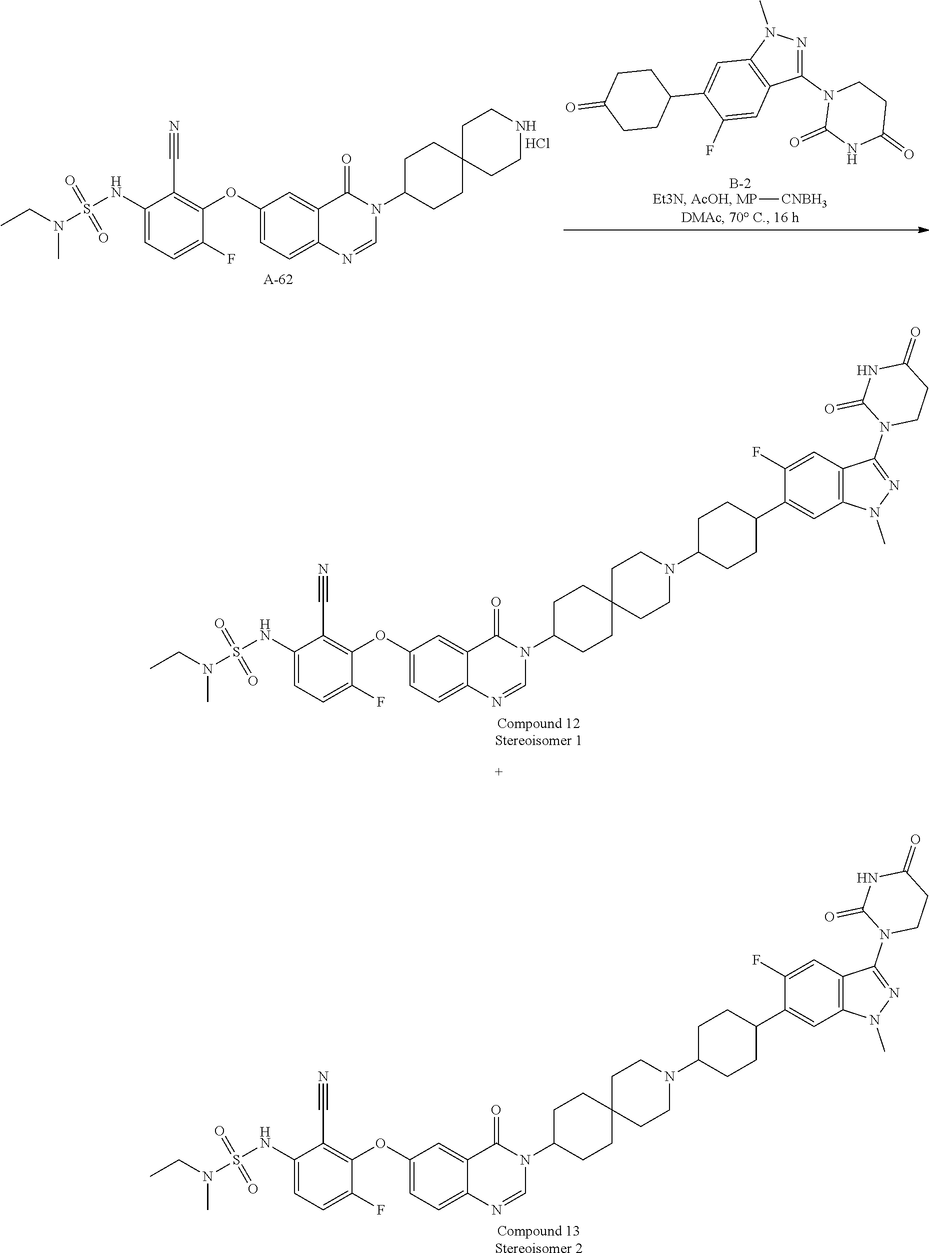

Into a 40 mL pressure vial containing a well-stirred solution of 9-[6-[2-cyano-3-[[ethyl(methyl)sulfamoyl]amino]-6-fluoro-phenoxy]-4-oxo-quinazolin-3-yl]-3-azaspiro[5.5]undecane (A-62) (450 mg, 600.87 mol, HCl salt) and 1-[5-fluoro-1-methyl-6-(4-oxocyclohexyl)indazol-3-yl]hexahydropyrimidine-2,4-dione (B-2) (539.95 mg, 1.20 mmol) in DMAc (4 mL) was added triethylamine (1.80 mmol, 251.25 μL) at room temperature and resulting mixture was stirred for 30 minutes at the same temperature. After 1 hour, AcOH (1.20 mmol, 68.80 L) was added at 0° C. and stirring was continued at room temperature for 1 hour. Subsequently, MP-CNBH₃ (600 mg) was added, and resulting reaction mixture was stirred at 70° C. for 16 hours. After the completion of the reaction, the reaction mixture was filtered through cotton pad and the filtrate was concentrated under reduced pressure to get a crude residue which was purified by reverse phase column chromatography (Column: Redisep Rf Gold® reversed-phase C18-teledyne ISCO, 120 g, Mobile phase A: 10 mm ammonium bicarbonate in milli-Q water; Mobile phase B: Acetonitrile). Fractions were collected and lyophilized to afford 350 mg mixture of cis and trans isomers. Further cis and trans isomers were separated by preparative HPLC (Column: X-BRIDGE C8 (150*19*5 u), Mobile phase A: 10 mm ammonium bicarbonate in water, Mobile phase B: acetonitrile, flow rate: 15 ml/min). Both fractions from HPLC were collected separately and lyophilized to get two isomers. Further these two isomers were analyzed by SFC and assigned as stereoisomer 1 (Peak-1 (t_{R}=2.65 min) by SFC sequence, Peak-2 by Prep HPLC sequence) and Stereoisomer 2 (Peak-2 (t_{R}=3.98 min) by SFC sequence, Peak-1 by Prep HPLC sequence).

Peak-1 (by SFC sequence): (9-[6-[2-cyano-3-[[ethyl(methyl)sulfamoyl]amino]-6-fluoro-phenoxy]-4-oxo-quinazolin-3-yl]-3-[4-[3-(2,4-dioxohexahydropyrimidin-1-yl)-5-fluoro-1-methyl-indazol-6-yl]cyclohexyl]-3-azaspiro[5.5]undecane (Compound 12, 51.40 mg, 55.27 mol, 15% yield) as a white solid. UPLC-MS (ES+): m/z 911.3 [M+H]+. ¹H NMR (400 MHz, DMSO-d₆): δ 10.57 (s, 1H), 8.48 (s, 1H), 7.75 (d, J=8.80 Hz, 1H), 7.62 (dd, J=2.80, 8.80 Hz, 1H), 7.50 (d, J=5.60 Hz, 1H), 7.42-7.34 (m, 3H), 7.28-7.26 (m, 1H), 4.60-4.49 (m, 1H), 4.04 (s, 3H), 3.92 (t, J=6.80 Hz, 2H), 3.65-3.35 (m, 2H), 3.22-3.04 (m, 2H), 3.00 (q, J=6.80 Hz, 2H), 2.68 (t, J=2.00 Hz, 2H), 2.58 (s, 3H), 2.26-1.74 (m, 11H), 1.71-1.40 (m, 7H), 1.30-1.15 (m, 1H), 1.03 (t, J=7.20 Hz, 3H).

Peak-2 (by SFC sequence): 9-[6-[2-cyano-3-[[ethyl(methyl)sulfamoyl]amino]-6-fluoro-phenoxy]-4-oxo-quinazolin-3-yl]-3-[4-[3-(2,4-dioxohexahydropyriimidin-1-yl)-5-fluoro-1-methyl-indazol-6-yl]cyclohexyl]-3-azaspiro[5.5]undecane (Compound 13, 45.60 mg, 49.76 mol, 13% yield) as a white solid. UPLC-MS (ES+): m/z 911.3 [M+H]+. ¹H NMR (400 MHz, DMSO-d₆): δ 10.56 (s, 1H), 8.94 (bs, 1H), 8.49 (s, 1H), 7.75 (d, J=8.80 Hz, 1H), 7.62 (dd, J=3.20, 9.00 Hz, 1H), 7.55 (d, J=6.00 Hz, 1H), 7.40-7.34 (m, 3H), 7.26-7.23 (m, 1H), 4.61-4.48 (m, 1H), 4.00 (s, 3H), 3.91 (t, J=6.80 Hz, 2H), 3.28-3.03 (m, 3H), 3.01-2.85 (m, 3H), 2.76 (t, J=6.80 Hz, 2H), 2.52 (s, 3H), 2.30-1.90 (m, 7H), 1.80-1.35 (m, 11H), 1.03 (t, J=4.40 Hz, 3H).

### Synthesis D8: Synthesis of 3-[(3aR,6aS)-2-[4-[3-(2,4-dioxohexahydropyrimidin-1-yl)-5-fluoro-1-methyl-indazol-6-yl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 14 and Compound 15)

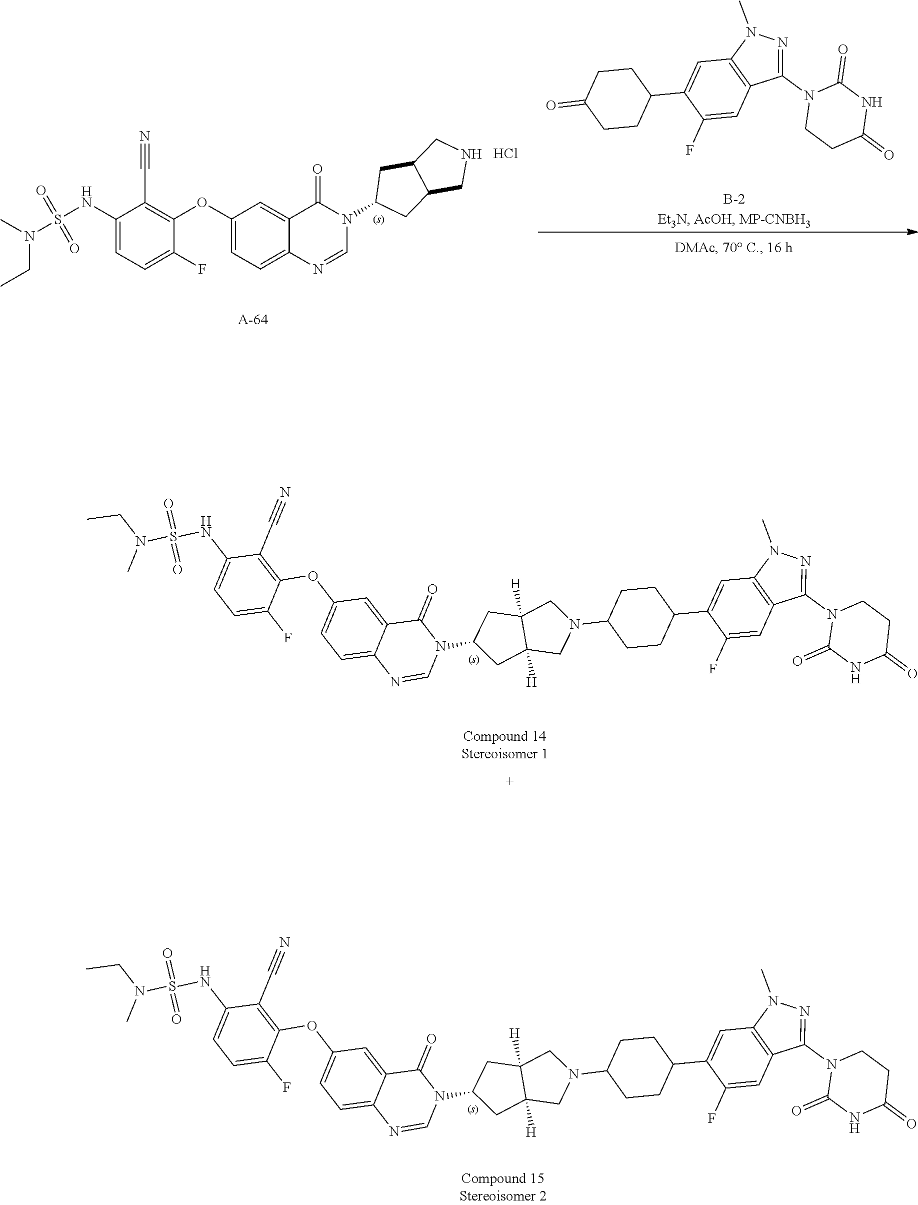 [SMILES: CCN(C)S(=O)(=O)Nc1ccc(F)c(Oc2ccc3ncn([C@@H]4C[C@H]5CNC[C@H]5C4)c(=O)c3c2)c1C#N ; Cl ; Cn1nc(N2CCC(=O)NC2=O)c2cc(F)c(C3CCC(=O)CC3)cc21 ; "A-64" ; CCN(C)S(=O)(=O)Nc1ccc(F)c(Oc2ccc3ncn([C@H]4C[C@@H]5CN(C6CCC(c7cc8c(cc7F)c(N7CCC(=O)NC7=O)nn8C)CC6)C[C@@H]5C4)c(=O)c3c2)c1C#N ; CCN(C)S(=O)(=O)Nc1ccc(F)c(Oc2ccc3ncn([C@H]4C[C@@H]5CN(C6CCC(c7cc8c(cc7F)c(N7CCC(=O)NC7=O)nn8C)CC6)C[C@@H]5C4)c(=O)c3c2)c1C#N]

Into a 20 mL pressure vial containing a well stirred solution of 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-64) (500 mg, 888.03 mol, HCl salt) in DMAc (10 mL) was added triethylamine (2.66 mmol, 371.32 μL) at room temperature and the resulting mixture was stirred for 5 min at the same temperature. After 5 minutes, AcOH (1.78 mmol, 101.67 μL) followed by 1-[5-fluoro-1-methyl-6-(4-oxocyclohexyl)indazol-3-yl]hexahydropyrimidine-2,4-dione (B-2) (636.48 mg, 1.78 mmol) were added at 0° C. and stirring was continued at room temperature for 1 hour. Subsequently, MP-CNBH₃ (1 g, 888.03 mol) was added, and resulting reaction mixture was stirred at 70° C. for 16 hours. After completion of the reaction, the reaction mixture was filtered through cotton pad and the filtrate was concentrated under reduced pressure to get a crude residue which was purified by reverse phase column chromatography (Column: Redisep Rf Gold® reversed-phase C18-teledyne ISCO, 100 g), [Mobile phase A: 10 mM ammonium bicarbonate in milli-Q water; Mobile phase B: acetonitrile]. The fractions were lyophilized to afford 500 mg of cis and trans isomeric mixture. Further cis and trans isomers were separated by preparative HPLC [Column: SHIM PACK (250*21.2*5 u), Mobile phase A: 10 mm NH₄HCO₃ in milli-Q water; Mobile phase B: acetonitrile]. Both fractions from HPLC were collected separately and lyophilized to get two isomers. Further these two isomers were analyzed by SFC and assigned as stereoisomer 1 (Peak-1 (t_{R}=4.55 min) by SFC sequence, Peak-2 by Prep HPLC sequence) and stereoisomer 2 (Peak-2 (t_{R}=6.93 min) by SFC sequence, Peak-1 by Prep HPLC sequence).

Peak-1 (by SFC sequence): 3-[(3aR,6aS)-2-[4-[3-(2,4-dioxohexahydropyrimidin-1-yl)-5-fluoro-1-methyl-indazol-6-yl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 14 27 mg, 30.45 mol, 3% yield, 98% purity) as an off-white solid. UPLC-MS (ES+): m/z 869.8 [M+H]+. ¹H NMR in VT (80° C.) (400 MHz, DMSO-d₆): δ 10.27 (s, 1H), 9.75 (bs, 1H), 8.35 (s, 1H), 7.74 (d, J=8.8 Hz, 1H), 7.67-7.60 (m, 2H), 7.46-7.43 (m, 3H), 7.38 (d, J=10.8 Hz, 1H), 4.91 (bs, 1H), 4.01 (s, 3H), 3.94 (t, J=6.4 Hz, 2H), 3.19-3.17 (q, J=6.8 Hz, 2H), 3.06-3.01 (m, 2H), 2.91-2.82 (m, 2H), 2.81-2.65 (m, 8H), 2.45-2.32 (m, 2H), 2.22-2.06 (m, 5H), 1.88-1.80 (m, 2H), 1.72-1.62 (m, 4H), 1.1 (t, J=6.8 Hz, 3H).

Peak-2 (by SFC sequence): 3-[(3aR,6aS)-2-[4-[3-(2,4-dioxohexahydropyrimidin-1-yl)-5-fluoro-1-methyl-indazol-6-yl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 15, 90 mg, 103.17 mol, 12% yield) as an off-white solid. Assigned as stereoisomer 2. UPLC-MS (ES+): m/z 869.8 [M+H]+. ¹H NMR (400 MHz, DMSO-d₆): δ 10.56 (s, 1H), 9.69 (bs, 1H), 8.46 (s, 1H), 7.78 (d, J=8.8 Hz, 1H), 7.65 (dd, J=2.8, 9.2 Hz, 1H), 7.56 (d, J=5.6 Hz, 1H), 7.41-7.27 (m, 4H), 5.05-4.90 (m, 1H), 4.01 (s, 3H), 3.91 (t, J=6.4 Hz, 2H), 3.80-3.45 (m, 1H), 3.24-3.08 (m, 2H), 3.03-2.80 (m, 5H), 2.75 (t, J=6.4 Hz, 3H), 2.60 (s, 3H), 2.35-2.15 (m, 4H), 2.10-1.77 (m, 4H), 1.73-1.48 (m, 4H), 1.0 (t, J=7.2 Hz, 3H).

### Synthesis D9: Synthesis of 3-[(3aR,6aS)-2-[4-[3-(2,4-dioxohexahydropyrimidin-1-yl)-5-fluoro-1-methyl-indazol-6-yl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 16 and Compound 17)

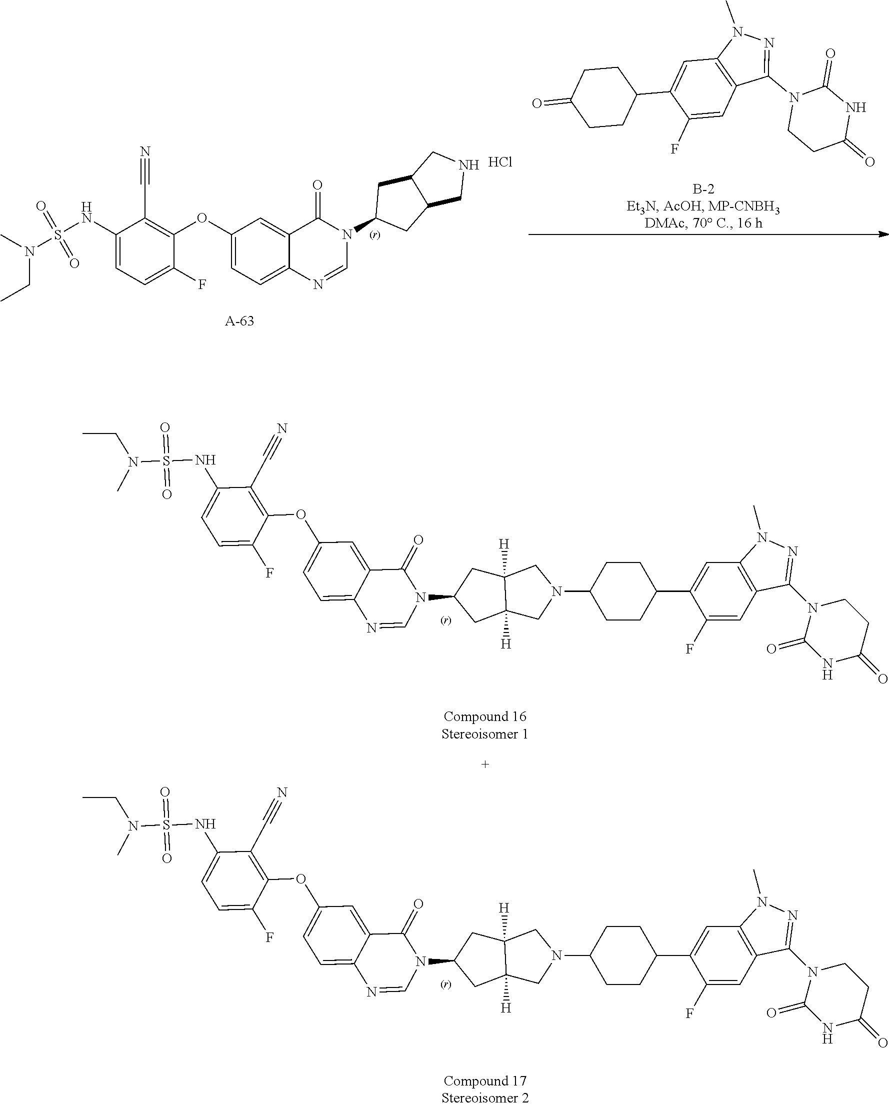

Into a 20 mL pressure vial containing a well stirred solution of 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-63) (500 mg, 710.43 mol, HCL salt) in DMAc (10 mL) was added triethylamine (2.13 mmol, 297.06 μL) at room temperature and stirred for 5 minutes. After 5 minutes, AcOH (710.43 mol, 40.67 μL) followed by 1-[5-fluoro-1-methyl-6-(4-oxocyclohexyl)indazol-3-yl]hexahydropyrimidine-2,4-dione (B-2) (763.78 mg, 2.13 mmol) were added at 0° C. and stirring was continued at room temperature for 2 hours. Subsequently, MP-CNBH₃ (1 g, 710.43 mol) was added, and resulting reaction mixture was stirred at 70° C. for 16 hours. After completion of the reaction, the reaction mixture was filtered through cotton pad and the filtrate was concentrated under reduced pressure to get a crude residue which was purified by reverse phase column chromatography (Column: Redisep R_{f}Gold® reversed-phase C18-teledyne ISCO, 100 g), [Mobile phase A: 10 mM ammonium bicarbonate in milli-Q water; Mobile phase B: acetonitrile]. Fractions were collected and lyophilized to afford 500 mg of cis and trans isomeric mixture. Further, these isomers were separated by preparative HPLC (Column: SHIM PACK (250 mm*21.2 mm*5 μm) and [Mobile phase A: 10 mm NH₄HCO₃ in milli-Q water; Mobile phase B: acetonitrile]. Both fractions from HPLC were lyophilized to get two isomers. Further these two isomers were analyzed by SFC and assigned as stereoisomer 1 (Peak-1 (t_{R}=4.32 min) by SFC sequence, Peak-2 by Prep HPLC sequence) and assigned as stereoisomer 2 (Peak-2 (t_{R}=7.83 min) by SFC sequence, Peak-1 by Prep HPLC sequence).

Peak-1 (by SFC sequence): 3-[(3aR,6aS)-2-[4-[3-(2,4-dioxohexahydropyrimidin-1-yl)-5-fluoro-1-methyl-indazol-6-yl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 16, 48.20 mg, 54.32 mol, 8% yield) as an off-white solid. Peak-1 was assigned as stereoisomer 1. UPLC-MS (ES+): m/z 869.8 [M+H]+. ¹H NMR (400 MHz, DMSO-d₆): δ 10.56 (s, 1H), 10.15 (bs, 1H), 8.43 (s, 1H), 7.87-7.37 (m, 7H), 5.53-5.20 (m, 1H), 4.10-3.80 (m, 6H), 3.13-2.90 (m, 4H), 2.80-2.68 (s, 7H), 2.33-2.10 (m, 4H), 2.09-1.40 (m, 11H), 1.09-0.9 (m, 3H).

Peak-2 (by SFC sequence): 3-[(3aR,6aS)-2-[4-[3-(2,4-dioxohexahydropyrimidin-1-yl)-5-fluoro-1-methyl-indazol-6-yl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 17, 38.20 mg, 42.84 mol, 6% yield) as an off-white solid. Peak-2 was assigned as stereoisomer 2. UPLC-MS (ES+): m/z 869.8 [M+H]+¹H NMR (400 MHz, DMSO-d₆): δ 10.55 (s, 1H), 9.80 (bs, 1H), 8.40 (s, 1H), 7.76 (d, J=8.80 Hz, 1H), 7.64-7.31 (m, 6H), 5.30-5.15 (m, 1H), 4.00 (s, 3H), 3.91 (t, J=6.40 Hz, 2H), 3.10-2.97 (m, 2H), 2.96-2.80 (m, 3H), 2.75 (t, J=7.20 Hz, 3H), 2.61 (s, 3H), 2.33-2.10 (m, 6H), 2.05-1.85 (m, 5H), 1.71-1.35 (m, 5H), 1.03 (t, J=7.20 Hz, 3H).

### Synthesis D10: Synthesis of 3-[(3aR,6aS)-2-[4-[4-[(2,6-dioxo-3-piperidyl)amino]-2-fluoro-phenyl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 18 and Compound 19)

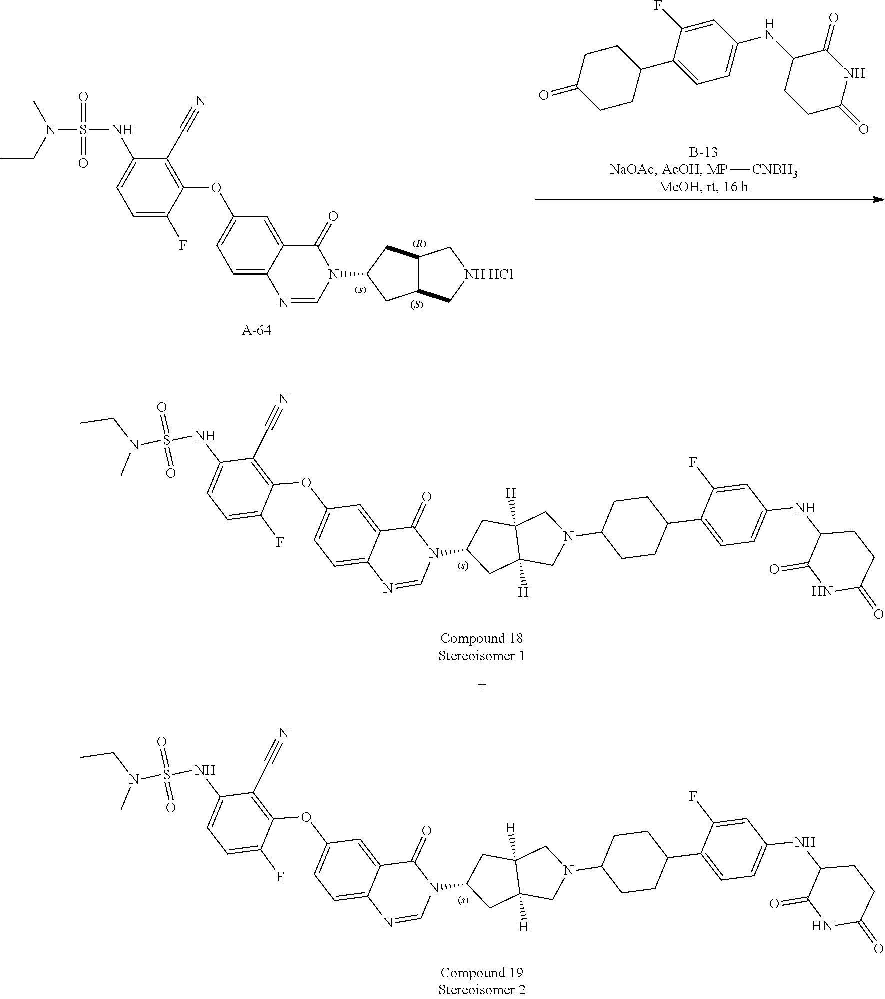

Into a 20 mL pressure vial containing a well stirred solution of 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-64) (500 mg, 783.42 mol, HCl salt) in methanol (10 mL) was added NaOAc (321.32 mg, 3.92 mmol) at room temperature and the resulting mixture was stirred for 5 minutes at the same temperature. After 5 minutes, AcOH (2.35 mmol, 134.54 μL) followed by 3-[3-fluoro-4-(4-oxocyclohexyl)anilino]piperidine-2,6-dione (B-13) (737.97 mg, 1.57 mmol) at room temperature and stirred for 30 minutes at the same temperature. Subsequently, MP-CNBH₃ (1 g, 783.42 mol) was added, and resulting reaction mixture was stirred at room temperature for 16 hours. After completion of the reaction, the reaction mixture was filtered through cotton pad and the filtrate was concentrated under reduced pressure to get a crude residue which was purified by prep HPLC (Column: PHENOMENEX GEMINI C18 (250 mm*21.2 mm*5 μm) and [Mobile phase A: 10 mm NH₄HCO₃ in milli-Q water; Mobile phase B: acetonitrile, flow rate: 15 mL/min]. Both fractions from HPLC were lyophilized to get two isomers. Further these two isomers were analyzed by SFC and assigned as stereoisomer 1 (Peak-1 (t_{R}=2.59 min) by SFC sequence, Peak-2 by Prep HPLC sequence) and assigned as stereoisomer 2 (Peak-2 (t_{R}=2.68 min) by SFC sequence, Peak-1 by Prep HPLC sequence).

Peak-1 (by SFC sequence): 3-[(3aR,6aS)-2-[4-[4-[(2,6-dioxo-3-piperidyl)amino]-2-fluoro-phenyl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 18, 65 mg, 77.10 mol, 10% yield) as an off-white solid. Peak-1 was assigned as stereoisomer 1. UP-LC-MS(ES+): m/z 829.3 [M+H]+. ¹H NMR in VT (80° C.) (400 MHz, DMSO-d₆): δ 10.53 (s, 1H), 9.70 (bs, 1H), 8.34 (s, 1H), 7.80 (d, J=8.0 Hz, 1H), 7.62 (dd, J=2.8, 9.0 Hz, 2H), 7.48-7.42 (m, 2H), 7.08 (t, J=8.4 Hz, 1H), 6.54 (dd, J=2.4, 8.4 Hz, 1H), 6.47 (dd, J=2.4, 13.8 Hz, 1H), 5.80-5.60 (m, 1H), 5.00-4.85 (m, 1H), 4.3-4.22 (m, 1H), 3.17-3.11 (m, 2H), 2.95-2.63 (m, 11H), 2.61-2.57 (m, 1H), 2.41-2.32 (m, 2H), 2.19-2.10 (m, 1H), 2.05-1.75 (m, 8H), 1.74-1.45 (m, 4H), 1.1 (t, J=7.2 Hz, 3H).

Peak-2 (by SFC sequence): 3-[(3aR,6aS)-2-[4-[4-[(2,6-dioxo-3-piperidyl)amino]-2-fluoro-phenyl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 19, 23 mg, 27.02 mol, 3% yield) as a white solid. Peak-2 was assigned as stereoisomer 2. UP-LC-MS(ES+): m/z 829.3 [M+H]+. ¹H NMR (400 MHz, DMSO-d₆): δ 10.79 (s, 1H), 9.75 (bs, 1H), 8.46 (s, 1H), 7.77 (d, J=8.8 Hz, 1H), 7.63 (dd, J=3.2, 9.0 Hz, 1H), 7.34-7.26 (m, 3H), 6.99 (t, J=9.2 Hz, 1H), 6.47-6.43 (m, 2H), 6.03 (d, J=7.6 Hz, 1H), 5.20-4.85 (m, 1H), 4.35-4.25 (m, 1H), 3.80-3.55 (m, 1H), 3.25-2.70 (m, 8H), 2.65-2.55 (m, 5H), 2.32-2.03 (m, 6H), 2.00-1.75 (m, 5H), 1.70-1.40 (m, 4H), 1.0 (t, J=7.2 Hz, 3H).

### Synthesis D11: Synthesis of 3-[(3aR,6aS)-2-[4-[4-[(2,6-dioxo-3-piperidyl)amino]-2-fluoro-phenyl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 20 and Compound 21)

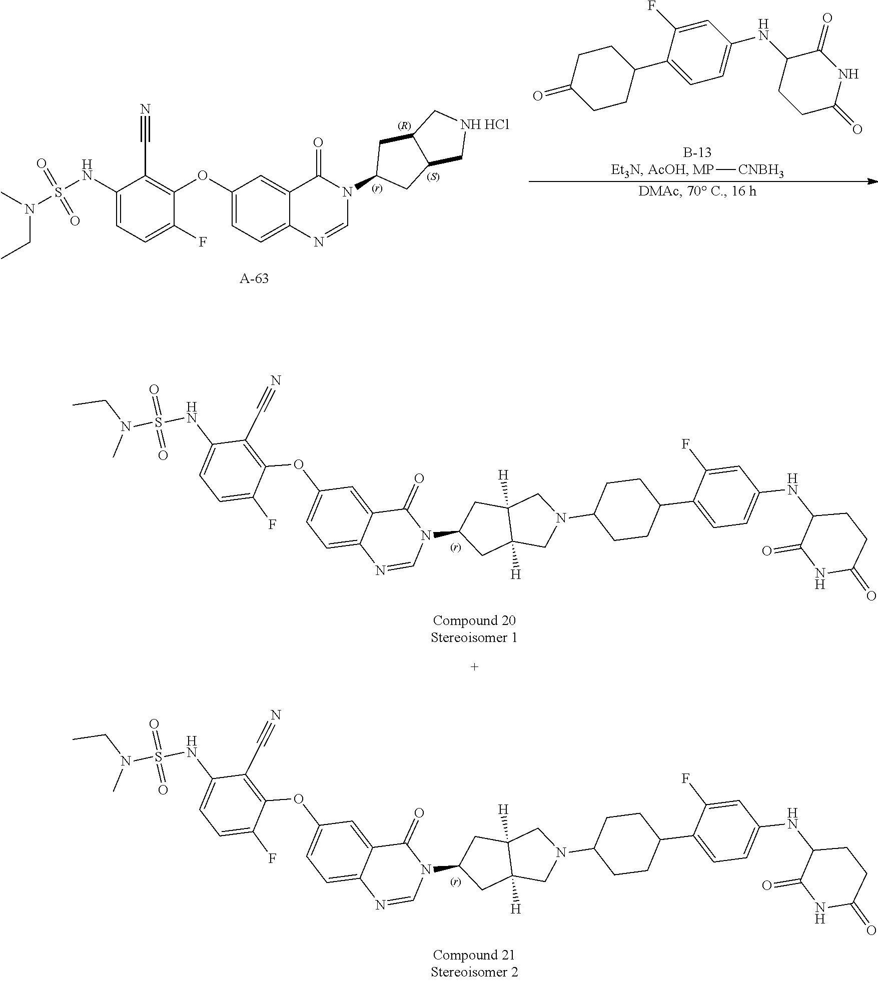

Into a 20 mL pressure vial containing a well stirred solution of 3-(1,2,3,3a,4,5,6,6a-octahydrocyclopenta[c]pyrrol-5-yl)-6-[2-cyano-3-[[ethyl(methyl)sulfamoyl]amino]-6-fluoro-phenoxy]-4-oxo-quinazoline (A-63) (500.00 mg, 888.03 mol, HCl salt) in DMAc (10 mL) was added triethylamine (2.66 mmol, 371.32 μL) at room temperature and the resulting mixture was stirred for 5 min at the same temperature. After 5 min, AcOH (2.66 mmol, 152.51 μL) followed by 3-[3-fluoro-4-(4-oxocyclohexyl)anilino]piperidine-2,6-dione (B-13) (848.09 mg, 2.66 mmol) were added at 0° C. and stirring was continued at room temperature for 2 hours. Subsequently, MP-CNBH₃ (1 g, 888.03 mol) was added, and resulting reaction mixture was stirred at 70° C. for 16 hours. After the completion of the reaction, the reaction mixture was filtered through cotton pad and the filtrate was concentrated under reduced pressure to get a crude residue which was purified by Preparative HPLC (Column: SHIM PACK (250 mm*21.2 mm*5 μm) and [Mobile phase A:10 mM NH₄HCO₃ in milli-Q water; Mobile phase B: acetonitrile]). Both fractions from HPLC were lyophilized to get two isomers. Further these two isomers were analyzed by SFC and assigned as stereoisomer 1 (Peak-1 (t_{R}=2.52 min) by SFC sequence, Peak-2 by Prep HPLC sequence) and assigned as stereoisomer 2 (Peak-2 (t_{R}=2.66 min) by SFC sequence, Peak-1 by Prep HPLC sequence).

Peak-1 (by SFC sequence): 3-[(3aR,6aS)-2-[4-[4-[(2,6-dioxo-3-piperidyl)amino]-2-fluoro-phenyl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 20, 36 mg, 42.35 mol, 5% yield) as an off-white solid. Peak-1 was assigned as stereoisomer 1. UP-LC-MS(ES+): m/z 829.3 [M+H]+. ¹H NMR (400 MHz, DMSO-d₆): δ 10.79 (s, 1H), 10.13 (bs, 1H), 8.39 (s, 1H), 7.75 (d, J=8.80 Hz, 1H), 7.61-7.16 (m, 5H), 6.52-6.42 (m, 2H), 6.10-5.80 (m, 1H), 5.50-5.10 (m, 1H), 4.35-4.25 (m, 1H), 4.10-3.80 (m, 1H), 3.60-3.40 (m, 1H), 3.10-2.98 (m, 3H), 2.95-2.85 (m, 1H), 2.80-2.65 (m, 7H), 2.30-2.17 (m, 2H), 2.15-2.07 (m, 2H), 2.05-1.70 (m, 9H), 1.68-1.35 (m, 3H), 1.02 (t, J=6.80 Hz, 3H).

Peak-2 (by SFC sequence): 3-[(3aR,6aS)-2-[4-[4-[(2,6-dioxo-3-piperidyl)amino]-2-fluoro-phenyl]cyclohexyl]-3,3a,4,5,6,6a-hexahydro-1H-cyclopenta[c]pyrrol-5-yl]-6-[2-cyano-3-[[ethyl(methyl)sulfamoyl]amino]-6-fluoro-phenoxy]-4-oxo-quinazoline (Compound 21, 30 mg, 35.37 mol, 4% yield) as an off-white solid. Peak-2 was assigned as stereoisomer 2. UP-LC-MS(ES+): m/z 829.3 [M+H]+. ¹H NMR (400 MHz, DMSO-d₆): δ 10.79 (s, 1H), 9.60 (bs, 1H), 8.32 (s, 1H), 7.75 (d, J=8.80 Hz, 1H), 7.61 (dd, J=2.80, 9.00 Hz, 1H), 7.35-7.50 (m, 2H), 7.32-7.20 (m, 1H), 6.98 (t, J=8.40 Hz, 1H), 6.47-6.43 (m, 2H), 6.02 (d, J=7.60 Hz, 1H), 5.25-5.15 (m, 1H), 4.35-4.25 (m, 1H), 3.80-3.40 (m, 1H), 3.25-3.10 (m, 1H), 3.08-2.97 (m, 2H), 2.95-2.81 (m, 2H), 2.79-2.70 (m, 1H), 2.60 (s, 3H), 2.45-2.35 (m, 3H), 2.30-2.20 (m, 2H), 2.19-2.02 (m, 3H), 2.00-1.75 (m, 6H), 1.70-1.30 (m, 5H), 1.02 (t, J=7.20 Hz, 3H).

### Synthesis D12: Synthesis of 6-[2-cyano-3-[[ethyl(methyl)sulfamoyl]amino]-6-fluoro-phenoxy]-3-[(3R)-8-[4-[4-[(2,6-dioxo-3-piperidyl)amino]-2-fluoro-phenyl]cyclohexyl]-8-azaspiro[4.5]decan-3-yl]-4-oxo-quinazoline (Compound 22 and Compound 23)

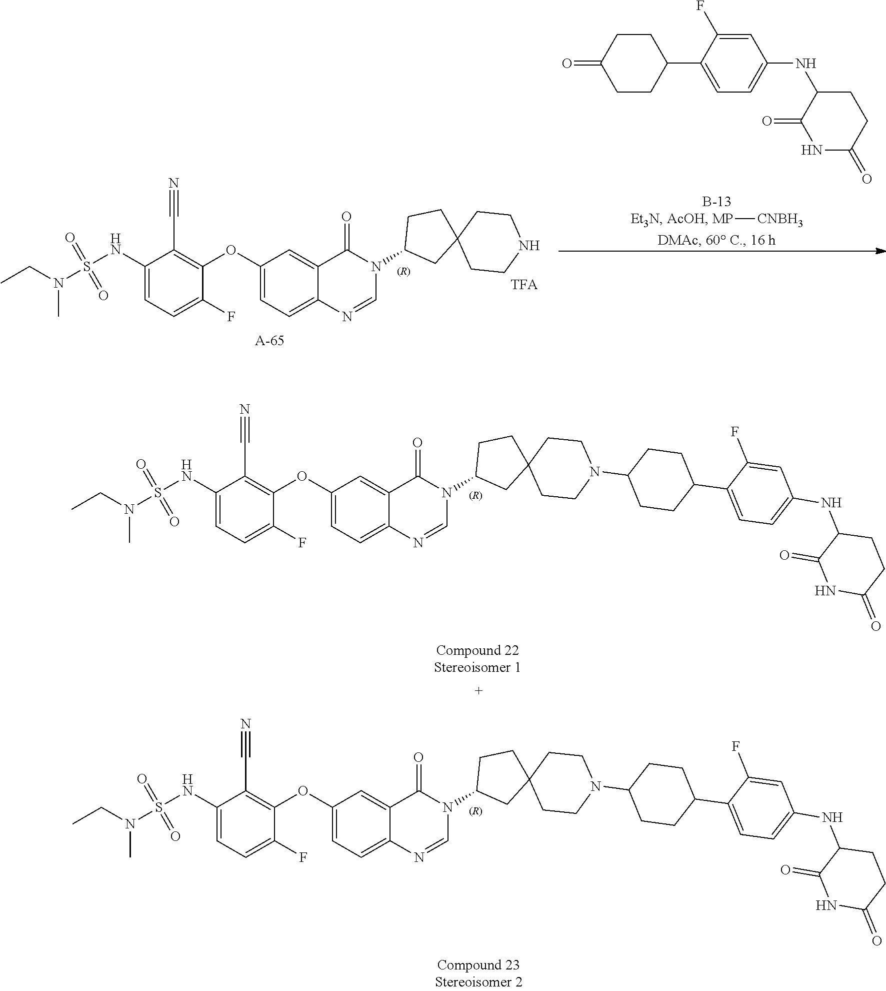

Into a 40 mL pressure vial containing a well-stirred solution of 3-[(3R)-8-azaspiro[4.5]decan-3-yl]-6-[2-cyano-3-[[ethyl(methyl)sulfamoyl]amino]-6-fluoro-phenoxy]-4-oxo-quinazoline (A-65) (0.5 g, 747.77 mol, TFA salt) in anhydrous DMAc (5.0 mL) were added triethylamine (3.74 mmol, 521.12 μL) and AcOH (2.24 mmol, 128.42 μL) and stirred at room temperature for 10 min. To the reaction mixture was added 3-[3-fluoro-4-(4-oxocyclohexyl)anilino]piperidine-2,6-dione (B-13) (476.09 mg, 1.50 mmol) and stirred at room temperature stirred for 1 hour. MP-CNBH₃ (1 g, 901.49 mol) was added, and the reaction mixture was stirred at 60° C. for 16 hours. After the completion of the reaction, the reaction mixture was filtered and the filtrate was concentrated under reduced pressure to get a crude residue which was purified by reverse phase column chromatography (Redisep Rf Gold®reversed-phase C18-teledyne ISCO, 120 g), [Mobile phase A: 10 mM NH₄HCO₃ in milli-Q water; Mobile phase B: acetonitrile). The fractions were lyophilized to afford 170 mg of mixture of cis and trans isomers. Further cis and trans isomers were separated by preparative HPLC [Column: X-select CSH-150 MM, Mobile phase A: 10 mm NH₄HCO₃ in milli-Q water; Mobile phase B: acetonitrile]. Both fractions from HPLC were lyophilized to get two isomers.

Further these two isomers were analyzed by SFC and assigned as stereoisomer 1 (Peak-1 (t_{R}=5.32 min) by SFC sequence, Peak-2 by Prep HPLC sequence) and assigned as stereoisomer 2 (Peak-2 (t_{R}=6.25 min) by SFC sequence, Peak-1 by Prep HPLC sequence).

Peak-1: (by SFC sequence): 6-[2-cyano-3-[[ethyl(methyl)sulfamoyl]amino]-6-fluoro-phenoxy]-3-[(3R)-8-[4-[4-[(2,6-dioxo-3-piperidyl)amino]-2-fluoro-phenyl]cyclohexyl]-8-azaspiro[4.5]decan-3-yl]-4-oxo-quinazoline (Compound 22, 28.22 mg, 32.70 mol, 18% yield) as an off-white solid. UPLC-MS (ES+): m/z 857.2 [M+H]+. ¹H NMR (400 MHz, DMSO-d₆): δ 10.52 (s, 1H), 8.35 (s, 1H), 7.74 (d, J=8.80 Hz, 1H), 7.59 (dd, J=3.20, 8.80 Hz, 1H), 7.44-7.35 (m, 3H), 7.07 (t, J=8.80 Hz, 1H), 6.52-6.45 (m, 2H), 5.79 (d, J=7.60 Hz, 1H), 5.10-4.90 (m, 1H), 4.35-4.25 (m, 1H), 3.00-2.87 (m, 3H), 2.80-2.53 (m, 10H), 2.15-2.10 (m, 4H), 1.95-1.62 (m, 16H), 1.07 (t, J=7.20 Hz, 3H).

Peak-2: (by SFC sequence): 6-[2-cyano-3-[[ethyl(methyl)sulfamoyl]amino]-6-fluoro-phenoxy]-3-[(3R)-8-[4-[4-[(2,6-dioxo-3-piperidyl)amino]-2-fluoro-phenyl]cyclohexyl]-8-azaspiro[4.5]decan-3-yl]-4-oxo-quinazoline (Compound 23, 21.13 mg, 24.41 mol, 13% yield) as an off-white solid. UPLC-MS (ES+): m/z 857.2 [M+H]+. ¹H NMR (400 MHz, DMSO-d₆): δ 10.79 (s, 1H), 8.40 (s, 1H), 7.75 (d, J=8.80 Hz, 1H), 7.63 (dd, J=2.80, 9.00 Hz, 1H), 7.38-7.23 (m, 3H), 6.97 (t, J=8.40 Hz, 1H), 6.46-6.42 (m, 2H), 6.02 (d, J=7.60 Hz, 1H), 5.03-4.98 (m, 1H), 4.33-4.27 (m, 1H), 3.20-3.05 (m, 3H), 2.98 (q, J=7.20 Hz, 2H), 2.71-2.60 (m, 1H), 2.59-2.56 (m, 5H), 2.15-2.06 (m, 6H), 1.91-1.71 (m, 10H), 1.69-1.49 (m, 6H), 1.02 (t, J=7.20 Hz, 3H).

### Synthesis D13: Synthesis of 6-[2-cyano-3-[[ethyl(methyl)sulfamoyl]amino]-6-fluoro-phenoxy]-3-[(3S)-8-[4-[4-[(2,6-dioxo-3-piperidyl)amino]-2-fluoro-phenyl]cyclohexyl]-8-azaspiro[4.5]decan-3-yl]-4-oxo-quinazoline (Compound 24 and Compound 25)

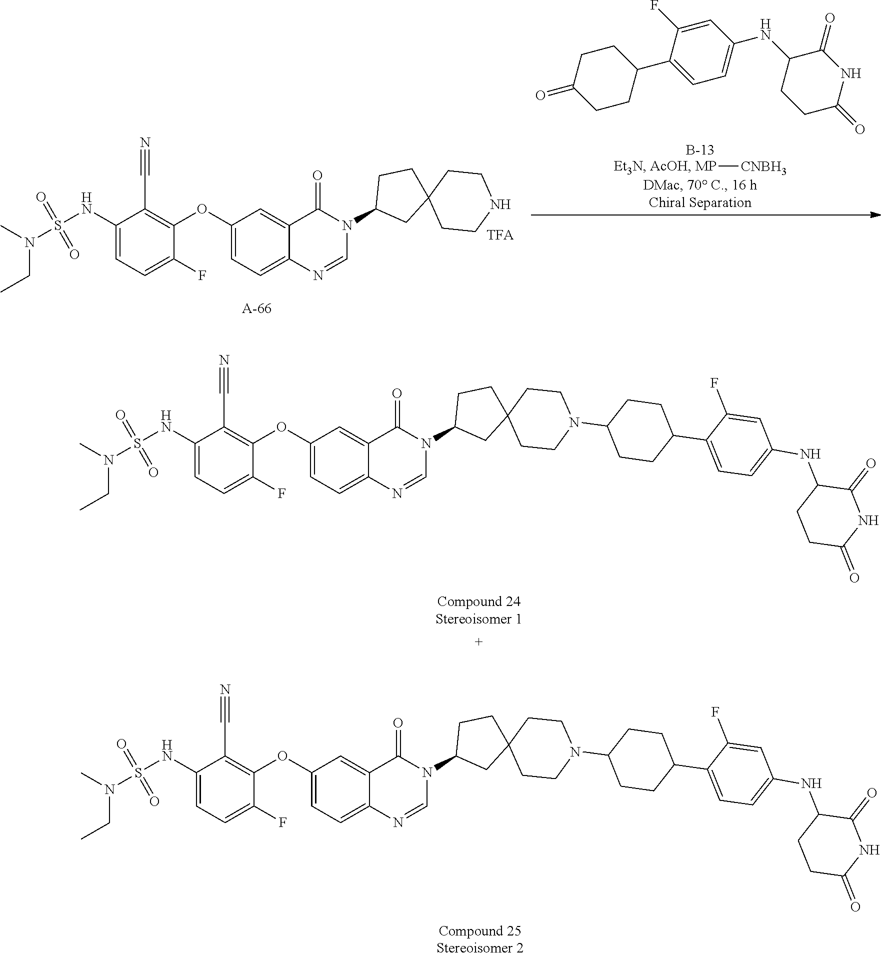

Into a 30 mL pressure vial containing a well stirred solution of 3-[(3S)-8-azaspiro[4.5]decan-3-yl]-6-[2-cyano-3-[[ethyl(methyl)sulfamoyl]amino]-6-fluoro-phenoxy]-4-oxo-quinazoline (A-66) (250.00 mg, 373.88 μmol, TFA Salt) in anhydrous DMAc (3 mL) was added triethylamine (1.87 mmol, 260.56 μL) stirred for 30 minutes. Added 3-[3-fluoro-4-(4-oxocyclohexyl)anilino]piperidine-2,6-dione (B-13) (119.02 mg, 373.88 μmol) followed by AcOH (1.12 mmol, 64.21 μL) and MP-CNBH₃ (0.400 g, 305.52 μmol) and the reaction mixture was stirred at 70° C. for 16 hours. After completion of the reaction, the reaction mixture was filtered and filtrate was concentrated under reduced pressure to get a crude residue which was purified by reverse phase column chromatography [Column: Redisep Rf Gold ® reversed-phase C18-teledyne ISCO, 120 g), Mobile phase A: 10 mM NH₄HCO₃ in milli-Q water; Mobile phase B: acetonitrile]. The fraction was lyophilized to afford mixture of cis and trans isomers 6-[2-cyano-3-[[ethyl(methyl)sulfamoyl]amino]-6-fluoro-phenoxy]-3-[(3S′)-8-[4-[4-[(2,6-dioxo-3-piperidyl)amino]-2-fluoro-phenyl]cyclohexyl]-8-azaspiro[4.5]decan-3-yl]-4-oxo-quinazoline (135 mg, 150.74 μmol, 40% yield) as an off white solid. Mixture of cis and trans isomers was separated by chiral SFC following the below method.

### Method of Analysis for Chiral SFC Separation

Instrument: PIC-175
Column: Diol(250*30)mm, 5 m
Mobile Phase: CO₂: 0.5% IPAm in IPA [60:40]
Total Flow: 120 ml/min
Back pressure: 100 bar
Wavelength: 220 nm
Cycle time: 16 min

Fast eluting isomer (Peak-1): Fast eluting compound (Peak-1) was collected and concentrated under reduced pressure to afford 6-[2-cyano-3-[[ethyl(methyl)sulfamoyl]amino]-6-fluoro-phenoxy]-3-[(3S)-8-[4-[4-[(2,6-dioxo-3-piperidyl)amino]-2-fluoro-phenyl]cyclohexyl]-8-azaspiro[4.5]decan-3-yl]-4-oxo-quinazoline (36 mg, 98.57% purity) as an off-white solid. Peak-1 was assigned as stereoisomer 1.

Late eluting isomer (Peak-2): Late eluting compound (Peak-2, t_{R}=4.68 min) was collected and concentrated under reduced pressure to afford 6-[2-cyano-3-[[ethyl(methyl)sulfamoyl]amino]-6-fluoro-phenoxy]-3-[(3S)-8-[4-[4-[(2,6-dioxo-3-piperidyl)amino]-2-fluoro-phenyl]cyclohexyl]-8-azaspiro[4.5]decan-3-yl]-4-oxo-quinazoline (35 mg, 98% purity) as an off-white solid. Peak-2 was assigned as stereoisomer 2.

As per ¹H-NMR, both stereoisomers contain little impurities, so further separated by reverse phase column chromatography (Column: Redisep Rf Gold® reversed-phase C18-teledyne ISCO, 120 g), Mobile phase A: 10 mM NH₄HCO₃ in milli-Q water; Mobile phase B: acetonitrile].

Further these two isomers were analyzed by SFC and assigned as stereoisomer 1 (Peak-1 (t_{R}=3.14 min) by SFC sequence, Peak-2 by Prep HPLC sequence) and assigned as stereoisomer 2 (Peak-2 (t_{R}=4.61 min) by SFC sequence, Peak-1 by Prep HPLC sequence).

First eluting fraction (Peak-1) was lyophilized to afford Compound 24 (assigned as stereoisomer 1).

Peak-1: 6-[2-cyano-3-[[ethyl(methyl)sulfamoyl]amino]-6-fluoro-phenoxy]-3-[(3S)-8-[4-[4-[(2,6-dioxo-3-piperidyl)amino]-2-fluoro-phenyl]cyclohexyl]-8-azaspiro[4.5]decan-3-yl]-4-oxo-quinazoline (Compound 24, 24 mg, 27.60 μmol, 18% yield) as an off-white solid. UPLC-MS (ES+): m/z 857.6 [M+H]+. ¹H NMR (400 MHz, DMSO-d₆): δ 10.79 (s, 1H), 8.40 (s, 1H), 7.75 (d, J=8.80 Hz, 1H), 7.62 (dd, J=3.20, 8.80 Hz, 1H), 7.49-6.98 (m, 4H), 6.50-6.44 (m, 2H), 6.08-5.98 (m, 1H), 5.19-4.89 (m, 1H), 4.39-4.23 (m, 1H), 3.41-3.62 (m, 2H), 3.19-2.89 (m, 4H), 2.71-2.81 (m, 1H), 2.65-2.57 (m, 4H), 2.24-2.01 (m, 4H), 2.00-1.57 (m, 16H), 1.03 (t, J=7.20 Hz, 3H).

Similarly, second eluted fraction (Peak-2) was lyophilized to afford Compound 25 (assigned as stereoisomer 2).

Peak-2: 6-[2-cyano-3-[[ethyl(methyl)sulfamoyl]amino]-6-fluoro-phenoxy]-3-[(3S)-8-[4-[4-[(2,6-dioxo-3-piperidyl)amino]-2-fluoro-phenyl]cyclohexyl]-8-azaspiro[4.5]decan-3-yl]-4-oxo-quinazoline (Compound 25, 22 mg, 25.08 μmol, 16% yield) as an off-white solid. UPLC-MS (ES+): m/z 857.6 [M+H]⁺. ¹H NMR (400 MHz, DMSO-d₆): δ 10.79 (s, 1H), 8.82 (bs, 1H), 8.40 (s, 1H), 7.75 (d, J=8.80 Hz, 1H), 7.62 (dd, J=2.80, 8.80 Hz, 1H), 7.33 (d, J=2.80 Hz, 2H), 7.28-7.15 (m, 1H), 7.02-6.89 (m, 1H), 6.49-6.37 (m, 2H), 6.05-5.95 (m, 1H), 4.88-5.08 (m, 1H), 4.25-4.35 (m, 1H), 3.06-2.91 (m, 3H), 2.58-2.55 (m, 3H), 2.20-2.03 (m, 6H), 1.79-1.95 (m, 7H), 1.64-1.43 (m, 5H), 1.02 (t, J=7.20 Hz, 3H).

### Synthesis D14: Synthesis of (3R)-3-[6-[2-cyano-3-[[ethyl(methyl)sulfamoyl]amino]-6-fluoro-phenoxy]-4-oxo-quinazolin-3-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 26)

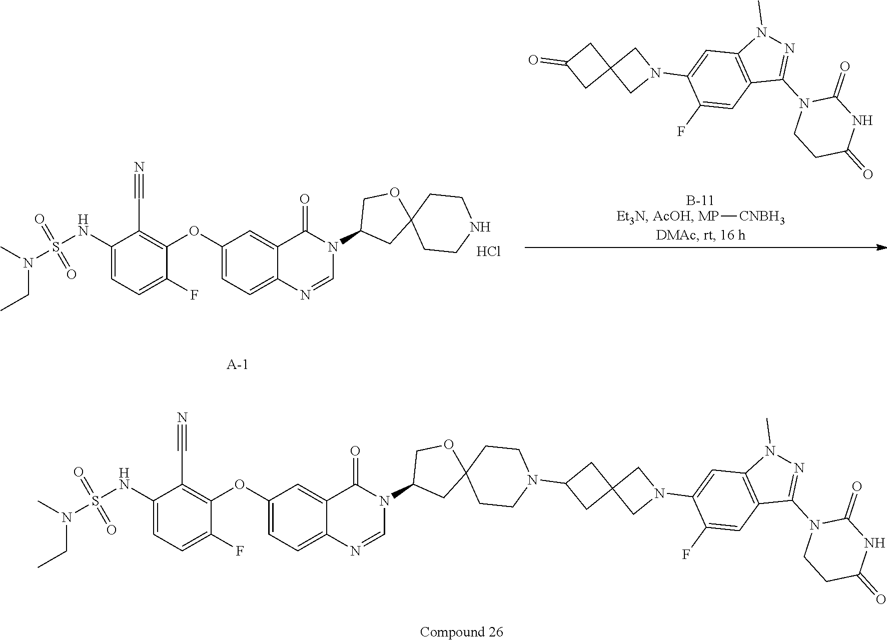

Into a pressure vial containing a well-stirred solution of (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1) (7 g, 11.80 mmol, HCl salt) in anhydrous DMAc (85 mL) was added triethylamine (59.02 mmol, 8.23 mL) at room temperature and the reaction mixture was stirred for 30 min. To the above mixture, 1-[5-fluoro-1-methyl-6-(6-oxo-2-azaspiro[3.3]heptan-2-yl)indazol-3-yl]hexahydropyrimidine-2,4-dione (B-11) (5.30 g, 12.98 mmol) and Acetic acid (35.41 mmol, 2.13 mL) were added at room temperature and mixture was stirred for 5 h. Subsequently, MP-CNBH₃ (7 g, 1.80 mmol) was added and resulting mixture was stirred at room temperature for 16 h. After the completion of the reaction, the reaction mixture was filtered through sintered funnel, washed with CH₂C12 and the filtrate was concentrated under reduced pressure to get a crude residue, which was purified by preparative HPLC (Column: X-Bridge C18 (150 mm*19 mm*5 μm), Mobile Phase A: 10 mM NH₄HCO₃ in water, Mobile Phase B: CH₃CN) to afford (3R)-3-[6-[2-cyano-3-[[ethyl(methyl)sulfamoyl]amino]-6-fluoro-phenoxy]-4-oxo-quinazolin-3-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 26, 2.45 g, 2.65 mmol, 22% yield) as an off-white solid.UIPLC-MS (ES+): m/z 912.7 [M+H]⁺. ¹H-NMR (400 MHz, DMSO-d₆): δ 10.50 (s, 1H), 9.60 (bs, 1H), 8.32 (s, 1H), 7.76 (d, J=8.80 Hz, 1H), 7.65 (dd, J=2.80, 8.80 Hz, 1H), 7.53 (bs, 1H), 7.40-7.21 (m, 3H), 6.51 (d, J=7.20 Hz, 1H), 5.35-5.21 (m, 1H), 4.21-4.00 (m, 4H), 3.98-3.78 (m, 7H), 3.20-2.80 (m, 5H), 2.79-2.69 (m, 4H), 2.65-2.61 (m, 3H), 2.61-2.55 (m, 3H), 2.47-2.36 (m, 2H), 2.32-1.97 (m, 2H), 1.96-1.49 (m, 3H), 1.03 (t, J=7.20 Hz, 3H) ppm.

### Synthesis D15: Synthesis of 6-[2-cyano-3-[[ethyl(methyl)sulfamoyl]amino]-6-fluoro-phenoxy]-3-[(3R)-8-[6-[3-(2,4-dioxohexahydropyrimidin-1-yl)-5-fluoro-1-methyl-indazol-6-yl]-2,6-diazaspiro[3.3]heptan-2-yl]-1-oxaspiro[4.5]decan-3-yl]-4-oxo-quinazoline (Compound 27)

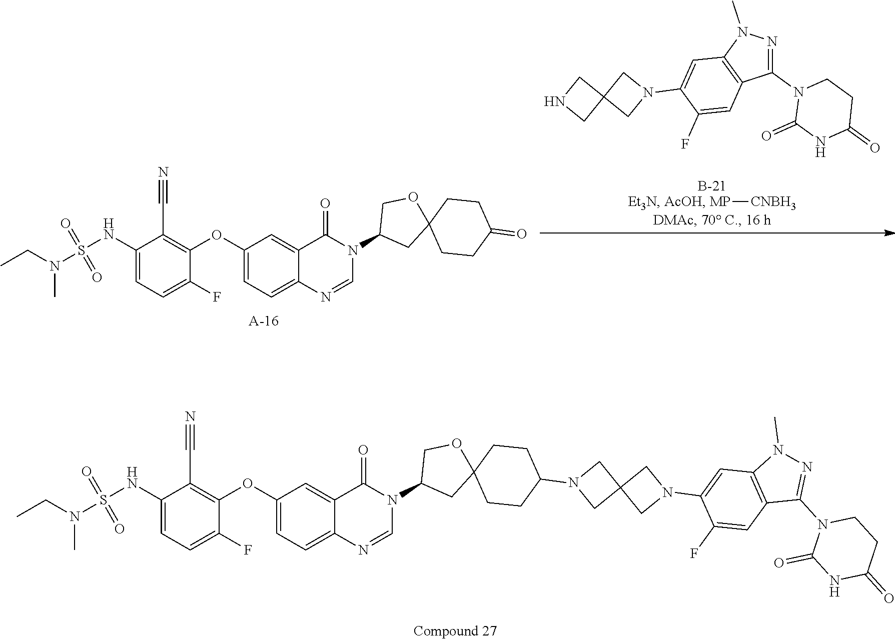

Into a 8 mL pressure capped vial containing a well-stirred solution of 6-[2-cyano-3-[[ethyl(methyl)sulfamoyl]amino]-6-fluoro-phenoxy]-4-oxo-3-[(3R)-8-oxo-1-oxaspiro[4.5]decan-3-yl]quinazoline (A-16) (108 mg, 117.56 μmol) and 1-[6-(2,6-diazaspiro[3.3]heptan-2-yl)-5-fluoro-1-methyl-indazol-3-yl]hexahydropyrimidine-2,4-dione (B-21) (73.62 mg, 132.47 μmol, TFA salt) in anhydrous DMAc (1.2 mL) were added triethylamine (587.78 μmol, 81.92 μL) and AcOH (352.67 μmol, 20.19 μL) at room temperature and the mixture was stirred for 5 hours. Subsequently, MP-CNBH₃ (200 mg, 117.56 μmol) was added, and mixture was stirred at 70° C. for 16 hours. After completion of the reaction, the reaction mixture was filtered through cotton pad and washed with dichloromethane and filtrate was concentrated under reduced pressure to get a crude residue, which was purified by reverse phase column chromatography (Column: Redisep Rf Gold® reversed-phase C18-teledyne ISCO, 120 g), mobile phase A: 10 mM ammonium bicarbonate in milli-Q water; mobile phase B: acetonitrile). Fractions containing pure compound were mixed and lyophilized to afford 6-[2-cyano-3-[[ethyl(methyl)sulfamoyl]amino]-6-fluoro-phenoxy]-3-[(3R)-8-[6-[3-(2,4-dioxohexahydropyrimidin-1-yl)-5-fluoro-1-methyl-indazol-6-yl]-2,6-diazaspiro[3.3]heptan-2-yl]-1-oxaspiro[4.5]decan-3-yl]-4-oxo-quinazoline (Compound 27), 20 mg, 21.40 μmol, 18% yield) as an off-white solid. UPLC-MS (ES+): m/z 912.2 [M+H]⁺. ¹H NMR (400 MHz, DMSO-d₆): δ 10.50 (d, J=1.60 Hz, 1H), 9.84 (bs, 1H), 8.31 (d, J=4.40 Hz, 1H), 7.74 (d, J=8.80 Hz, 1H), 7.61 (dd, J=2.80, 9.00 Hz, 1H), 7.42 (bs, 1H), 7.33 (d, J=2.40 Hz, 1H), 7.27 (dd, J=4.40, 12.80 Hz, 2H), 6.54 (t, J=6.40 Hz, 1H), 5.31-5.25 (quint, J=7.2 Hz, 1H), 4.50-4.21 (m, 2H), 4.19-4.02 (m, 6H), 3.93-3.85 (m, 5H), 3.01 (d, J=6.80 Hz, 2H), 2.72 (t, J=6.80 Hz, 2H), 2.63-2.55 (m, 3H), 2.43-2.35 (m, 1H), 2.31-2.25 (m, 1H), 2.09-1.63 (m, 5H), 1.61-1.52 (m, 1H), 1.48-1.29 (m, 2H), 1.28-1.12 (m, 1H), 1.03 (t, J=7.20 Hz, 3H).

### Synthesis D16: Synthesis of (3R)-3-[6-[2-cyano-3-[[ethyl(methyl)sulfamoyl]amino]-6-fluoro-phenoxy]-4-oxo-quinazolin-3-yl]-8-[(1R,4S)-4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 28 and Compound 29)

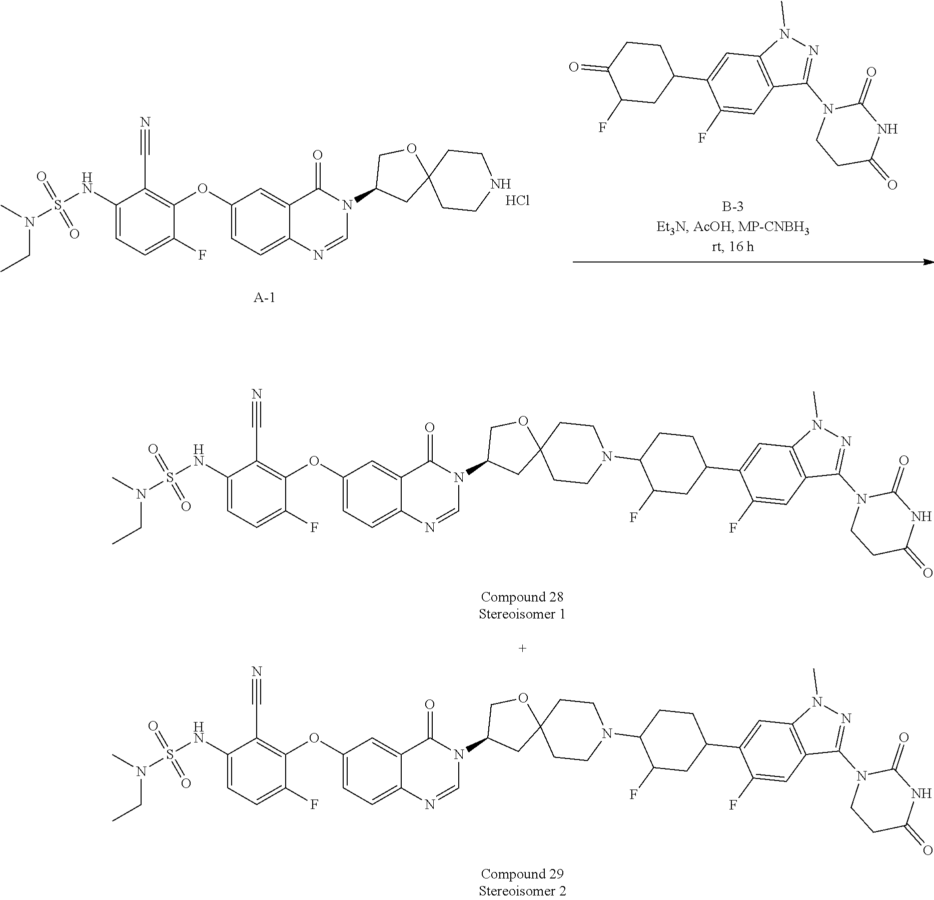 [SMILES: CCN(C)S(=O)(=O)Nc1ccc(F)c(Oc2ccc3ncn([C@H]4COC5(CCNCC5)C4)c(=O)c3c2)c1C#N ; Cl ; Cn1nc(N2CCC(=O)NC2=O)c2cc(F)c(C3CCC(=O)C(F)C3)cc21 ; CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6F)CC5)C4)c(=O)c3c2)c1C#N ; CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6F)CC5)C4)c(=O)c3c2)c1C#N]

Into an 8 mL glass vial with pressure released cap containing a well-stirred solution of (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1) (500.00 mg, 843.07 mol, HCl salt) and 1-[5-fluoro-6-(3-fluoro-4-oxo-cyclohexyl)-1-methyl-indazol-3-yl]hexahydropyrimidine-2,4-dione (B-3) (317.30 mg, 843.07 mol) in DMAc (5 mL) was added triethylamine (2.53 mmol, 352.52 μL) at room temperature and the resulting mixture was stirred for 1 h at the same temperature. After 1 h, AcOH (843.07 mol, 48.26 μL) was added at 0° C. and stirring was continued at room temperature for 2 hours. Subsequently, MP-CNBH₃ (1.0 g, 843.07 mol) was added, and resulting reaction mixture was stirred at room temperature for 16 hours. After the completion of the reaction, the reaction mixture was filtered through cotton pad and the filtrate was concentrated under reduced pressure to get a crude residue which was purified by reverse phase column chromatography (Column: Redisep Rf Gold® reversed-phase C18-teledyne ISCO, 120 g), [Mobile phase A: 0.1% FA in milli-Q water; Mobile phase B: acetonitrile]. Fractions were collected and lyophilized to afford a mixture of cis and trans isomers. Further cis and trans isomers were separated by preparative HPLC (Column: Shimpack C18(250 mm*21.2 mm*5 μm), mobile phase: 10 mm ABC_ACN). Both fractions from HPLC were concentrated separately and lyophilized to get two isomers. Further these two isomers were analyzed by SFC and assigned as stereoisomer 1 (Peak-1 (t_{R}=1.80 min) by SFC sequence, Peak-2 by Prep HPLC sequence) and stereoisomer 2 (Peak-2 (t_{R}=2.82 min) by SFC sequence, Peak-1 by Prep HPLC sequence).

Peak-1 (by SFC sequence): (3R)-3-[6-[2-cyano-3-[[ethyl(methyl)sulfamoyl]amino]-6-fluoro-phenoxy]-4-oxo-quinazolin-3-yl]-8-[(1R,4S)-4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 28, 34 mg, 35.79 mol, 4% yield). UPLC-MS (ES+): m/z 917.2 [M+H]⁺. ¹H NMR (400 MHz, DMSO-d₆): δ 10.55 (s, 1H), 10.16 (bs, 1H), 8.35 (s, 1H), 7.85-7.66 (m, 3H), 7.52 (d, J=5.60 Hz, 1H), 7.45-7.36 (m, 3H), 5.28-5.10 (m, 2H), 4.15-4.08 (m, 2H), 4.06 (s, 3H), 3.91 (t, J=6.80 Hz, 2H), 3.06-3.18 (m, 2H), 2.75 (t, J=6.80 Hz, 4H), 2.37-2.40 (m, 1H), 2.03-1.67 (m, 12H), 1.05 (t, J=7.20 Hz, 3H).

Peak-2 (by SFC sequence): (3R)-3-[6-[2-cyano-3-[[ethyl(methyl)sulfamoyl]amino]-6-fluoro-phenoxy]-4-oxo-quinazolin-3-yl]-8-[(1S,4S)-4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 29, 11 mg, 11.96 mol, 1% yield). UPLC-MS (ES+): m/z 917.2 [M+H]⁺. ¹H NMR (400 MHz, DMSO-d₆): δ 10.56 (s, 1H), 10.09 (bs, 1H), 8.34 (s, 1H), 7.78-7.58 (m, 4H), 7.41-7.35 (m, 3H), 5.25-5.32 (m, 2H), 4.15-4.11 (m, 2H), 3.99 (s, 3H), 3.90 (t, J=6.80 Hz, 2H), 3.07 (bs, 2H), 2.75 (t, J=6.80 Hz, 2H), 1.68-2.25 (m, 12H), 1.04 (t, J=7.20 Hz, 3H).

### Synthesis D17: Synthesis of 6-[2-cyano-3-[[ethyl(methyl)sulfamoyl]amino]-6-fluoro-phenoxy]-3-[(3R)-8-[6-[4-[(2,6-dioxo-3-piperidyl)amino]-2-fluoro-phenyl]-2,6-diazaspiro[3.3]heptan-2-yl]-1-oxaspiro[4.5]decan-3-yl]-4-oxo-quinazoline (Compound 30)

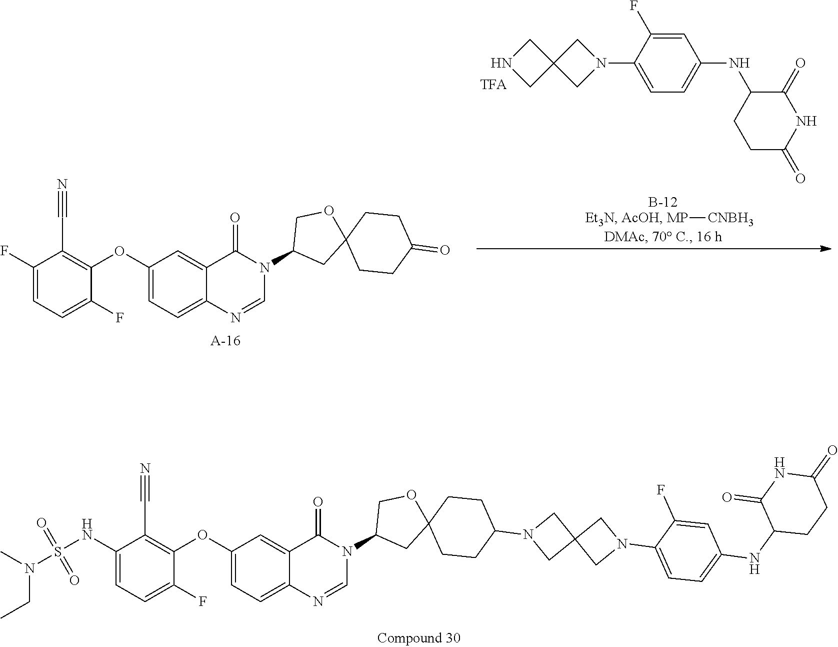

Into a 20 mL pressure vial containing a well-stirred solution of 6-[2-cyano-3-[[ethyl(methyl)sulfamoyl]amino]-6-fluoro-phenoxy]-4-oxo-3-[(3R)-8-oxo-1-oxaspiro[4.5]decan-3-yl]quinazoline (A-16) (100 mg, 175.56 mol) in DMAc (2.5 mL) was added triethylamine (877.80 mol, 122.35 μL) at room temperature under nitrogen atmosphere and the reaction mixture was stirred for 20 min. Reaction mixture was cooled to 0° C. and was added 3-[4-(2,6-diazaspiro[3.3]heptan-2-yl)-3-fluoro-anilino]piperidine-2,6-dione (B-12) (151.81 mg, 351.12 mol, TFA salt) followed by AcOH (526.68 mol, 30.15 μL) and reaction mixture was stirred for 3 h at room temperature. Finally, MP-CNBH₃ (200 mg, 175.56 mol) was added, and the reaction mixture was stirred at 70° C. for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fraction was lyophilized to afford 6-[2-cyano-3-[[ethyl(methyl)sulfamoyl]amino]-6-fluoro-phenoxy]-3-[(3R)-8-[6-[4-[(2,6-dioxo-3-piperidyl)amino]-2-fluoro-phenyl]-2,6-diazaspiro[3.3]heptan-2-yl]-1-oxaspiro[4.5]decan-3-yl]-4-oxo-quinazoline (Compound 30, 13 mg, 14.80 mol, 8.43% yield, 99.24% purity) as an off-white solid. UPLC-MS (ES+): m/z 872.3 [M+H]⁺. ¹H NMR (400 MHz, DMSO-d₆): δ 10.75 (s, 1H), 8.30 (d, J=4.00 Hz, 1H), 7.75 (d, J=9.20 Hz, 1H), 7.63 (dd, J=2.40, 8.80 Hz, 1H), 7.34-7.26 (m, 3H), 6.52-6.38 (m, 3H), 5.59 (t, J=6.40 Hz, 1H), 5.40-5.17 (m, 1H), 4.24-4.04 (m, 5H), 3.92-3.69 (m, 5H), 3.02-2.91 (m, 2H), 2.62-2.57 (m, 4H), 2.19-1.99 (m, 2H), 1.93-1.69 (m, 5H), 1.61-1.52 (m, 1H), 1.49-1.16 (m, 7H), 1.03 (t, J=4.00 Hz, 3H).

### Synthesis D18: Synthesis of 6-[2-cyano-3-[[ethyl(methyl)sulfamoyl]amino]-6-fluoro-phenoxy]-3-[(3R)-8-[4-[3-(2,4-dioxohexahydropyrimidin-1-yl)-5-fluoro-1-methyl-indazol-6-yl]cyclohexyl]-8-azaspiro[4.5]decan-3-yl]-4-oxo-quinazoline (Compound 31 and Compound 32)

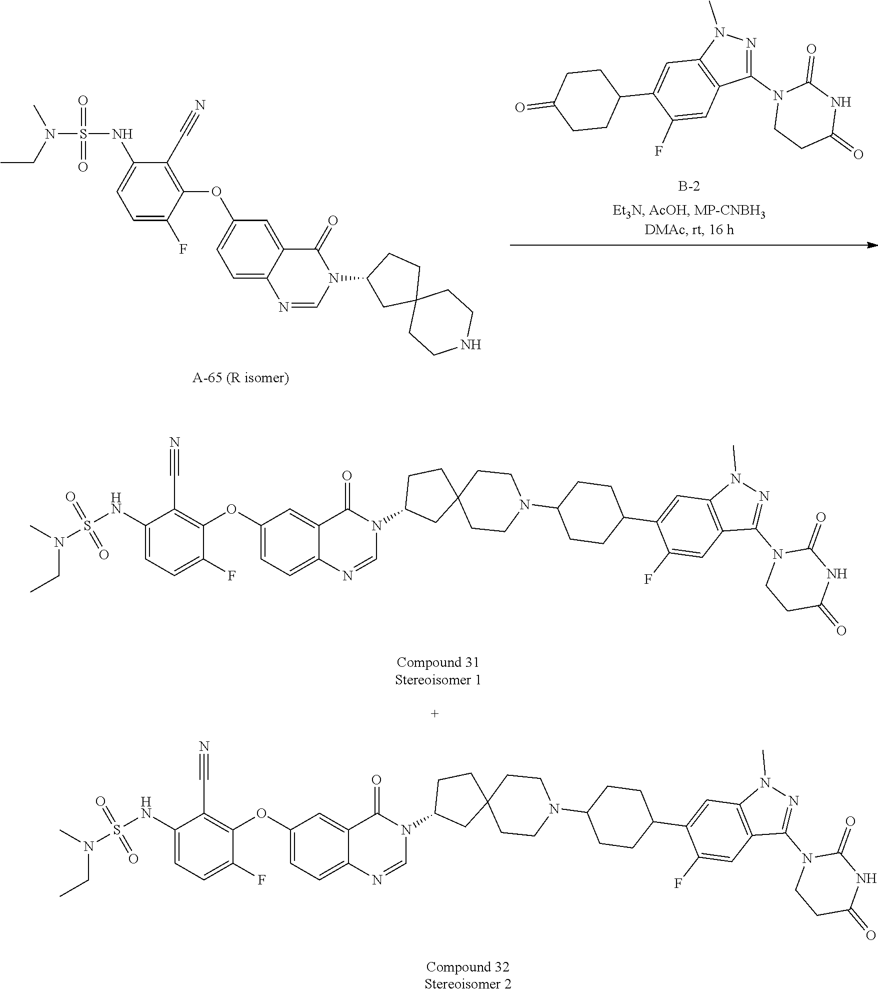

Into a 40 mL pressure vial containing a well-stirred solution of 3-[(3R)-8-azaspiro[4.5]decan-3-yl]-6-[2-cyano-3-[[ethyl(methyl)sulfamoyl]amino]-6-fluoro-phenoxy]-4-oxo-quinazoline (A-65) (0.50 g, 901.49 mol) in DMAc (5 mL) was added triethylamine (4.51 mmol, 628.25 μL) at room temperature under nitrogen atmosphere and stirred for 20 min. Reaction mixture was cooled to 0° C. and was added 1-[5-fluoro-1-methyl-6-(4-oxocyclohexyl)indazol-3-yl]hexahydropyrimidine-2,4-dione (B-2) (484.60 mg, 1.35 mmol) followed by AcOH (2.70 mmol, 154.82 μL) and resulting mixture was stirred at room temperature for 3 hours. MP-CNBH₃ (1 g, 901.49 mol) was added, and the reaction mixture was stirred at room temperature for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fractions were lyophilized to afford 210 mg of mixture of cis and trans isomers. Further cis and trans isomers were separated by preparative HPLC [Column: SHIMPACK C18 (250 mm*21.2 mm*5 m), Mobile phase A: 10 mM NH₄HCO₃ in milli-Q water; Mobile phase B: acetonitrile]. Further these two isomers were analyzed by SFC and assigned as stereoisomer 1 (Peak-1 (t_{R}=3.6 min) by SFC sequence, Peak-2 by Prep HPLC sequence) and stereoisomer 2 (Peak-2 (t_{R}=5.5 min) by SFC sequence, Peak-1 by Prep HPLC sequence). First eluting fraction (Peak-1) was lyophilized to afford Compound 31 (assigned as stereoisomer 1).

Peak-1 (by SFC sequence): 6-[2-cyano-3-[[ethyl(methyl)sulfamoyl]amino]-6-fluoro-phenoxy]-3-[(3R)-8-[4-[3-(2,4-dioxohexahydropyrimidin-1-yl)-5-fluoro-1-methyl-indazol-6-yl]cyclohexyl]-8-azaspiro[4.5]decan-3-yl]-4-oxo-quinazoline (Compound 31, 53.94 mg, 59.56 mol, 24% yield) as an off-white solid. UPLC-MS (ES+): m/z 897.4 [M+H]⁺. ¹H NMR (400 MHz, DMSO-d₆): δ 10.56 (s, 1H), 8.62 (bs, 1H), 8.41 (s, 1H), 7.75 (d, J=9.20 Hz, 1H), 7.63 (dd, J=3.20, 9.20 Hz, 1H), 7.48 (d, J=6.00 Hz, 1H), 7.41-7.13 (m, 4H), 5.12-4.88 (m, 1H), 4.03 (s, 3H), 3.91 (t, J=6.80 Hz, 2H), 3.69-3.40 (m, 2H), 3.23-2.83 (m, 5H), 2.76 (t, J=6.80 Hz, 2H), 2.59 (s, 3H), 2.26-1.52 (m, 18H), 1.03 (t, J=7.20 Hz, 3H)

Similarly, the second eluted fraction (Peak-2) was lyophilized to afford Compound 32 (assigned as stereoisomer 2).

Peak-2 (by SFC sequence): 6-[2-cyano-3-[[ethyl(methyl)sulfamoyl]amino]-6-fluoro-phenoxy]-3-[(3R)-8-[4-[3-(2,4-dioxohexahydropyrimidin-1-yl)-5-fluoro-1-methyl-indazol-6-yl]cyclohexyl]-8-azaspiro[4.5]decan-3-yl]-4-oxo-quinazoline (Compound 32, 24.7 mg, 27.23 mol, 11% yield) as an off-white solid. UPLC-MS (ES+): m/z 897.3 [M+H]⁺. ¹H NMR (400 MHz, DMSO-d₆): δ 10.56 (s, 1H), 8.95 (bs, 1H), 8.41 (s, 1H), 7.76 (d, J=8.80 Hz, 1H), 7.63 (dd, J=3.20, 9.20 Hz, 1H), 7.54 (d, J=6.00 Hz, 1H), 7.42-7.05 (m, 4H), 5.01 (quint, J=9.2 Hz, 1H), 4.00 (s, 3H), 3.91 (t, J=6.80 Hz, 2H), 3.20-2.82 (m, 6H), 2.75 (t, J=6.80 Hz, 2H), 2.57 (s, 3H), 2.29-1.60 (m, 18H), 1.03 (t, J=7.20 Hz, 3H).

### Synthesis D19: Synthesis of 6-[2-cyano-3-[[ethyl(methyl)sulfamoyl]amino]-6-fluoro-phenoxy]-3-[(3S)-8-[4-[3-(2,4-dioxohexahydropyrimidin-1-yl)-5-fluoro-1-methyl-indazol-6-yl]cyclohexyl]-8-azaspiro[4.5]decan-3-yl]-4-oxo-quinazoline (Compound 33 and Compound 34)

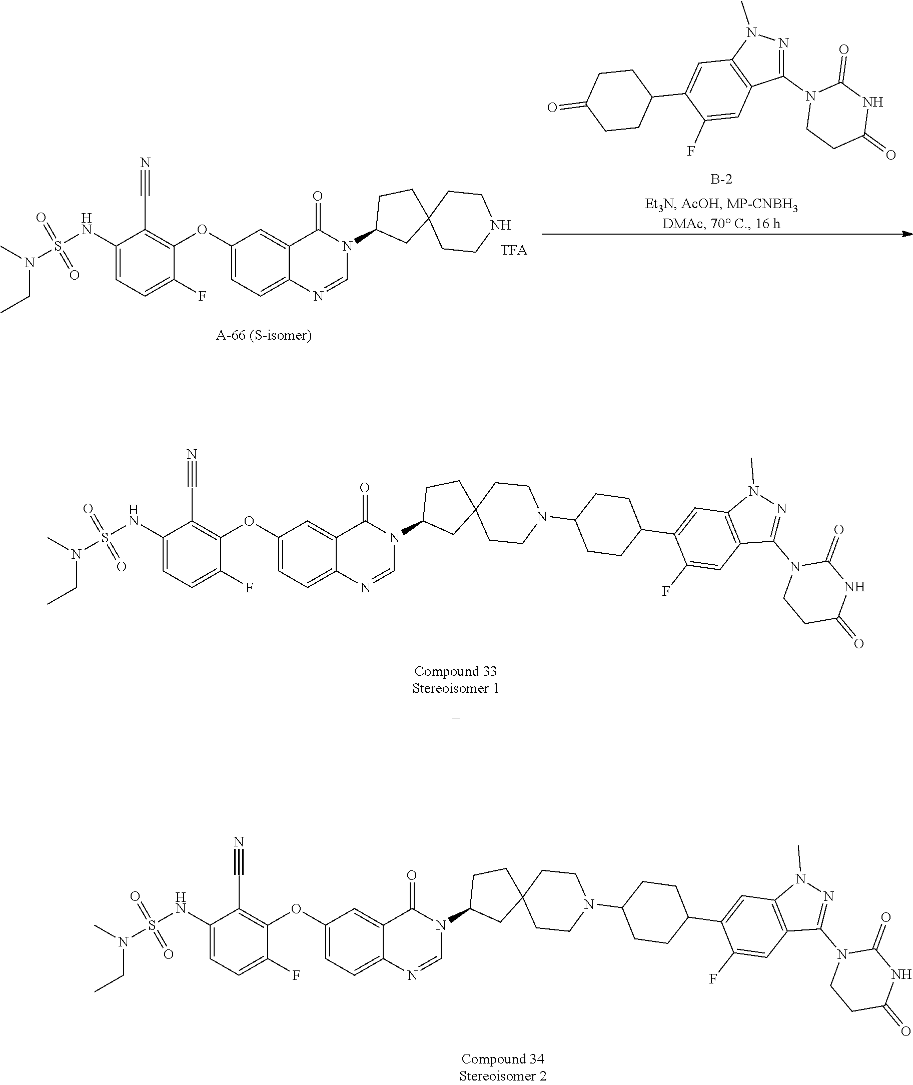

Into a 40 mL pressure vial containing a well-stirred solution 3-[(3S)-8-azaspiro[4.5]decan-3-yl]-6-[2-cyano-3-[[ethyl(methyl)sulfamoyl]amino]-6-fluoro-phenoxy]-4-oxo-quinazoline (A-66) (250 mg, 373.88 mol, TFA salt) in DMAc (5 mL) was added triethylamine (1.87 mmol, 260.56 μL) at room temperature under nitrogen atmosphere and mixture was stirred for 20 min. Reaction mixture was cooled to 0° C. and was added 1-[5-fluoro-1-methyl-6-(4-oxocyclohexyl)indazol-3-yl]hexahydropyrimidine-2,4-dione (B-2) (200.98 mg, 560.82 mol) followed by AcOH (1.12 mmol, 64.21 μL) and resulting mixture was stirred for 3 hours at room temperature. MP-CNBH₃ (500 mg, 373.88 mol) was added, and the reaction mixture was stirred at 70° C. for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fractions were lyophilized to afford 70 mg of mixture of cis and trans isomers. Further cis and trans isomers were separated by preparative HPLC. [Column: SHIMPACK C18 (250 mm*21.2 mm*5 m), Mobile phase A: 10 mM NH₄HCO₃ in milli-Q water; Mobile phase B: acetonitrile]. Further these two isomers were analyzed by SFC and assigned as stereoisomer 1 (Peak-1 (t_{R}=2.42 min) by SFC sequence, Peak-2 by Prep HPLC sequence) and stereoisomer 2 (Peak-2 (t_{R}=3.62 min) by SFC sequence, Peak-1 by Prep HPLC sequence). First eluting fraction (Peak-1) was lyophilized to afford Compound 33 (assigned as stereoisomer 1).

Peak-1 (by SFC sequence): 6-[2-cyano-3-[[ethyl(methyl)sulfamoyl]amino]-6-fluoro-phenoxy]-3-[(3S)-8-[4-[3-(2,4-dioxohexahydropyrimidin-1-yl)-5-fluoro-1-methyl-indazol-6-yl]cyclohexyl]-8-azaspiro[4.5]decan-3-yl]-4-oxo-quinazoline (Compound 33, 15 mg, 16.68 mol, 4% yield) as an off-white solid. UPLC-MS (ES+): m/z 897.2 [M+H]⁺. ¹H NMR (400 MHz, DMSO-d₆): δ 10.56 (s, 1H), 8.62 (bs, 1H), 8.41 (s, 1H), 7.75 (d, J=8.80 Hz, 1H), 7.63 (dd, J=2.80, 8.80 Hz, 1H), 7.48 (d, J=5.60 Hz, 1H), 7.46-7.16 (m, 4H), 5.11-4.90 (m, 1H), 4.03 (s, 3H), 3.91 (t, J=6.40 Hz, 2H), 3.69-3.39 (m, 1H), 3.22-2.81 (m, 5H), 2.76 (t, J=6.80 Hz, 2H), 2.59 (s, 3H), 2.30-1.39 (m, 20H), 1.03 (t, J=7.20 Hz, 3H).

Similarly, the second eluted fraction (Peak-2) was lyophilized to afford Compound 34 (assigned as stereoisomer 2).

Peak-2 (by SFC sequence): 6-[2-cyano-3-[[ethyl(methyl)sulfamoyl]amino]-6-fluoro-phenoxy]-3-[(3S)-8-[4-[3-(2,4-dioxohexahydropyrimidin-1-yl)-5-fluoro-1-methyl-indazol-6-yl]cyclohexyl]-8-azaspiro[4.5]decan-3-yl]-4-oxo-quinazoline (Compound 34, 5 mg, 5.46 mol, 1% yield). UPLC-MS (ES+): m/z 897.2 [M+H]⁺. ¹H NMR (400 MHz, DMSO-d₆): δ 10.56 (s, 1H), 8.43 (s, 1H), 7.78 (d, J=8.80 Hz, 1H), 7.66 (dd, J=3.20, 9.20 Hz, 1H), 7.54 (d, J=6.00 Hz, 1H), 7.47-7.31 (m, 3H), 5.03 (quint, J=8.80 Hz, 1H), 3.99 (s, 3H), 3.90 (t, J=6.80 Hz, 2H), 3.11-2.98 (m, 3H), 2.97-2.88 (m, 1H), 2.75 (t, J=6.80 Hz, 2H), 2.70-2.67 (m, 2H), 2.29-1.59 (m, 18H), 1.04 (t, J=7.20 Hz, 3H).

### Synthesis D20: Synthesis of (3R)-3-[6-[2-cyano-3-[[ethyl(methyl)sulfamoyl]amino]-6-fluoro-phenoxy]-4-oxo-quinazolin-3-yl]-8-[2-[4-[(2,6-dioxo-3-piperidyl)amino]-2-fluoro-phenyl]-2-azaspiro[3.3]heptan-6-yl]-1-oxa-8-azaspiro[4.5]decane (Compound 35)

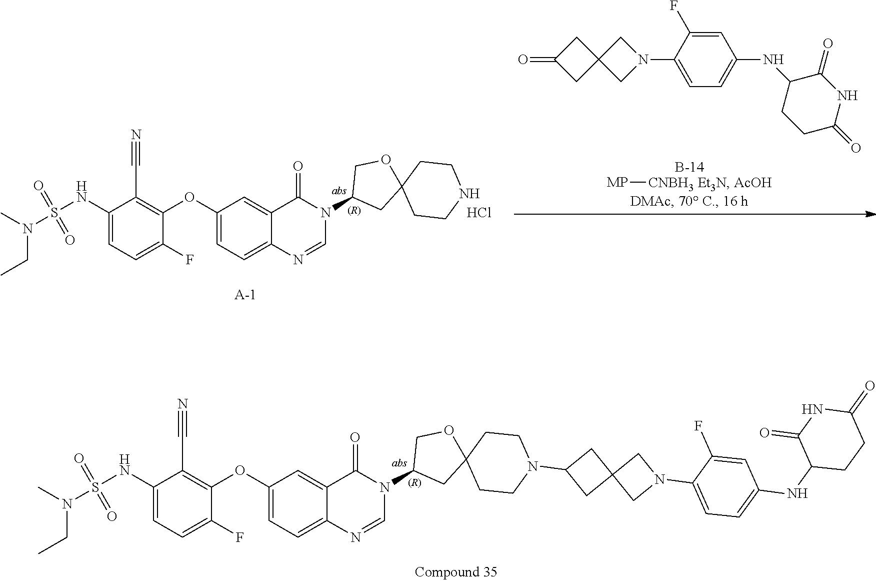

Into a 40 mL pressure vial containing a well-stirred solution of (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1) (400.00 mg, 667.71 mol, HCl salt) in DMAc (5 mL) was added triethylamine (2.00 mmol, 279.20 μL) at room temperature under nitrogen atmosphere and mixture was stirred for 20 min. Reaction mixture was cooled to 0° C. and was added 3-[3-fluoro-4-(6-oxo-2-azaspiro[3.3]heptan-2-yl)anilino]piperidine-2,6-dione (B-14) (265.49 mg, 596.12 mol, TFA salt) followed by AcOH (1.00 mmol, 57.33 μL). The resulting mixture was stirred for 3 h at room temperature. Finally, MP-CNBH₃ (400 mg, 334.09 mol) was added, and the reaction mixture was stirred at 70° C. for 16 hours. After the completion of the reaction, the reaction mixture was filtered and the filtrate was concentrated under reduced pressure to get a crude residue which was purified by reverse phase column chromatography (Redisep Rf Gold®reversed-phase C18-teledyne ISCO, 120 g), [Mobile phase A: NH₄HCO₃ in milli-Q water; Mobile phase B: acetonitrile]. The fractions were lyophilized and further separated by SFC. [Column: Diol (250*30)mm, 5 μm, Mobile phase A: CO₂: IPA (60:40)]. The fractions were lyophilized to afford (3R)-3-[6-[2-cyano-3-[[ethyl(methyl)sulfamoyl]amino]-6-fluoro-phenoxy]-4-oxo-quinazolin-3-yl]-8-[2-[4-[(2,6-dioxo-3-piperidyl)amino]-2-fluoro-phenyl]-2-azaspiro[3.3]heptan-6-yl]-1-oxa-8-azaspiro[4.5]decane (Compound 35, 40 mg, 43.69 mol, 7% yield) as a grey solid. UPLC-MS (ES+): m/z 872.3 [M+H]⁺. ¹H NMR (400 MHz, DMSO-d₆): δ 10.75 (s, 1H), 8.31 (s, 1H), 7.76 (d, J=8.80 Hz, 1H), 7.64 (dd, J=2.80, 8.80 Hz, 1H), 7.56-7.23 (m, 3H), 6.59-6.21 (m, 4H), 5.64-5.48 (m, 1H), 5.39-5.22 (m, 1H), 4.28-4.08 (m, 4H), 3.82-3.73 (m, 2H), 3.70-3.61 (m, 2H), 3.09-2.99 (m, 3H), 2.72-2.61 (m, 6H), 2.45-2.38 (m, 2H), 2.28-1.95 (m, 5H), 1.95-1.58 (m, 6H), 1.09-1.01 (m, 3H).

### Synthesis D21: Synthesis of 6-[2-cyano-3-[[ethyl(methyl)sulfamoyl]amino]-6-fluoro-phenoxy]-3-[(3R)-8-[(4S)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 36, Compound 37), 6-[2-cyano-3-[[ethyl(methyl)sulfamoyl]amino]-6-fluoro-phenoxy]-3-[(3R)-8-[(4R)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 38, Compound 39)

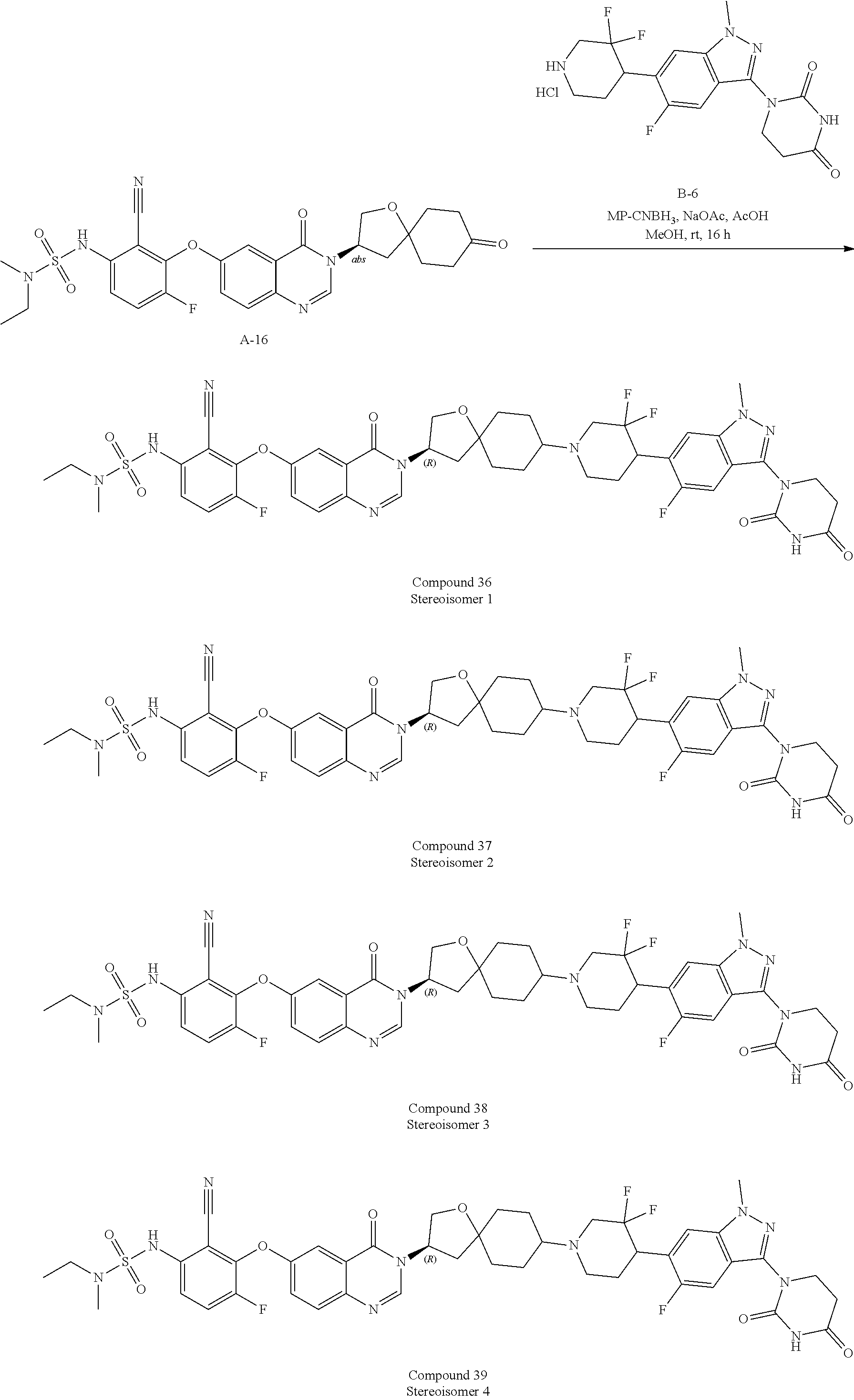

Into a 50 mL single-neck round-bottom flask containing a well-stirred solution of 1-[6-(3,3-difluoro-4-piperidyl)-5-fluoro-1-methyl-indazol-3-yl]hexahydropyrimidine-2,4-dione (B-6) (300 mg, 718.03 mol, HCl salt) dissolved in methanol (20 mL) was added anhydrous NaOAc (117.80 mg, 1.44 mmol) followed by 6-[2-cyano-3-[[ethyl(methyl)sulfamoyl]amino]-6-fluoro-phenoxy]-4-oxo-3-[(3R)-8-oxo-1-oxaspiro[4.5]decan-3-yl]quinazoline (A-16) (490.79 mg, 861.63 mol) and AcOH (71.80 mol, 4.11 μL) at 0° C. The resulting reaction mixture was stirred at room temperature for 1 hour. Subsequently, MP-CNBH₃ (500 mg, 718.03 mol) was added, and the resulting mixture was stirred at room temperature for 16 hours. After completion of the reaction, the reaction mixture was filtered through filter paper, and solid resin was washed with methanol (5 mL). The combined filtrate was concentrated under reduced pressure to get a crude residue which was purified by reverse phase column chromatography (Column: Redisep Rf Gold® reversed-phase C18-teledyne ISCO, 120 g), [Mobile phase A: 10 mM ammonium bicarbonate in milli-Q water; Mobile phase B: acetonitrile]. The fractions were collected and lyophilized to afford 185 mg of a mixture of isomers. Further isomers were separated by chiral SFC following the below method.

### Method of Analysis for Chiral SFC Separation

Instrument: PIC 175
Column: I CELLULOSE J-(250*30)mm, 5 m
Mobile Phase: CO₂: methanol: MeCN(1:1)[80:20]
Total Flow: 120 mL/min
Back pressure: 100 bar
Wavelength: 220 nm
Cycle time: 55 min

First eluting isomer (Peak-1): The first eluting fraction (Peak-1) at R_{f}=13.15 min was concentrated under reduced pressure at 40° C. and lyophilized to afford 6-[2-cyano-3-[[ethyl(methyl)sulfamoyl]amino]-6-fluoro-phenoxy]-3-[(3R)-8-[(4S)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 36, 23.7 mg, 25.25 mol, 4% yield) as an off-white solid. Peak-1 was assigned as stereoisomer 1. UPLC-MS (ES+): m/z 935.2 [M+H]⁺. ¹H NMR (400 MHz, DMSO-d₆): δ 10.56 (s, 1H), 10.18 (s, 1H), 8.34 (s, 1H), 7.89-7.55 (m, 4H), 7.46-7.32 (m, 3H), 5.27 (bs, 1H), 4.21-4.10 (m, 2H), 4.05 (s, 3H), 3.92 (t, J=6.40 Hz, 2H), 3.60-3.36 (m, 4H), 3.21-2.99 (m, 4H), 2.79-2.70 (m, 4H), 2.59-2.55 (m, 1H), 2.45-2.40 (m, 1H), 2.29-2.22 (m, 1H), 2.02-1.83 (m, 4H), 1.71-1.55 (m, 5H), 1.42-1.32 (m, 1H), 1.05 (t, J=7.20 Hz, 3H).

Second eluting isomer (Peak-2): The second eluted fraction (Peak-2) at Rₜ=14.59 min was concentrated under reduced pressure at 40° C. and lyophilized to afford 6-[2-cyano-3-[[ethyl(methyl)sulfamoyl]amino]-6-fluoro-phenoxy]-3-[(3R)-8-[(4S)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 37, 20 mg, 21.20 mol, 4% yield) as an off-white solid. Peak-2 was assigned as stereoisomer 2. UPLC-MS (ES+): m/z 935.3 [M+H]⁺. ¹H NMR (400 MHz, DMSO-d₆): δ 10.56 (s, 1H), 10.18 (s, 1H), 8.34 (s, 1H), 7.89-7.55 (m, 4H), 7.46-7.32 (m, 3H), 5.27 (bs, 1H), 4.21-4.10 (m, 2H), 4.05 (s, 3H), 3.92 (t, J=6.40 Hz, 2H), 3.60-3.36 (m, 4H), 3.21-2.99 (m, 4H), 2.79-2.70 (m, 4H), 2.59-2.55 (m, 2H), 2.35-2.22 (m, 1H), 2.02-1.83 (m, 4H), 1.71-1.55 (m, 5H), 1.42-1.32 (m, 1H), 1.05 (t, J=7.20 Hz, 3H).

Third eluting isomer (Peak-3): The third eluted fraction (Peak-3) at R_{f}=17.69 min was concentrated under reduced pressure at 40° C. and lyophilized to afford 6-[2-cyano-3-[[ethyl(methyl)sulfamoyl]amino]-6-fluoro-phenoxy]-3-[(3R)-8-[(4R)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 38, 18.43 mg, 18.95 mol, 3% yield) as an off-white solid. Peak-3 was assigned as stereoisomer 3. UPLC-MS (ES+): m/z 935.3 [M+H]⁺. ¹H NMR (400 MHz, DMSO-d₆): δ 10.56 (s, 1H), 10.18 (s, 1H), 8.36 (s, 1H), 7.89-7.60 (m, 4H), 7.46-7.32 (m, 3H), 5.26 (bs, 1H), 4.16-4.06 (m, 2H), 4.02 (s, 3H), 3.91 (t, J=6.80 Hz, 2H), 3.51-3.42 (m, 1H), 3.23-3.00 (m, 4H), 2.85-2.73 (m, 5H), 2.65-2.55 (m, 2H), 2.46-2.39 (m, 2H), 2.31-2.21 (m, 1H), 2.06-1.98 (m, 1H), 1.92-1.71 (m, 5H), 1.63-1.55 (m, 1H), 1.47-1.31 (m, 3H), 1.05 (t, J=7.20 Hz, 3H).

Fourth eluting isomer (Peak-4): Similarly, the fourth eluted fraction (Peak-4) at Rₜ=22.10 min was concentrated under reduced pressure at 40° C. and lyophilized to afford 6-[2-cyano-3-[[ethyl(methyl)sulfamoyl]amino]-6-fluoro-phenoxy]-3-[(3R)-8-[(4R)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 39, 25.5 mg, 25.63 mol, 4% yield) as an off-white solid. Peak-4 was assigned as stereoisomer 4. UPLC-MS (ES+): m/z 935.3 [M+H]⁺. ¹H NMR (400 MHz, DMSO-d₆): δ 10.56 (s, 1H), 10.17 (s, 1H), 8.35 (s, 1H), 7.79-7.64 (m, 4H), 7.42-7.36 (m, 3H), 5.27 (bs, 1H), 4.18-4.06 (m, 2H), 4.02 (s, 3H), 3.91 (t, J=6.80 Hz, 2H), 3.60-3.42 (m, 1H), 3.28-3.15 (m, 2H), 3.11-2.98 (m, 3H), 2.80-2.71 (m, 2H), 2.70-2.62 (m, 4H), 2.61-2.56 (m, 1H), 2.48-2.40 (m, 1H), 2.31-2.22 (m, 1H), 2.08-1.98 (m, 1H), 1.90-1.75 (m, 5H), 1.68-1.55 (m, 1H), 1.48-1.29 (m, 3H), 1.04 (t, J=7.20 Hz, 3H).

### Synthesis D22: Synthesis of 6-[2-cyano-3-[[ethyl(methyl)sulfamoyl]amino]-6-fluoro-phenoxy]-3-[(3R)-8-[4-[4-[(2,6-dioxo-3-piperidyl)amino]-2-fluoro-phenyl]piperazin-1-yl]-1-oxaspiro[4.5]decan-3-yl]-4-oxo-quinazoline (Compound 40)

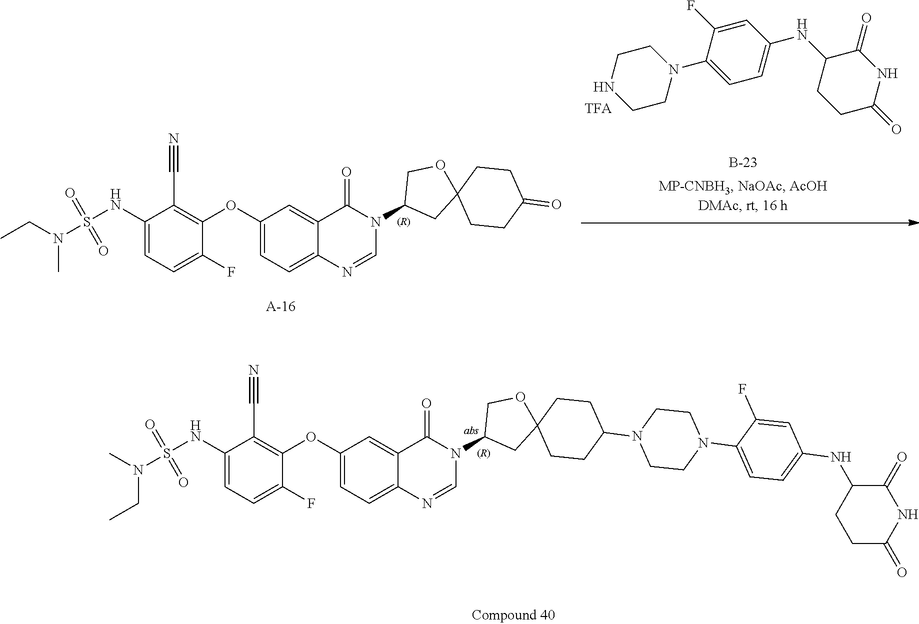

Into a 20 mL pressure vial containing a well stirred solution of 3-(3-fluoro-4-piperazin-1-yl-phenyl)piperidine-2,6-dione (B-23) (0.4 g, 1.37 mmol, TFA salt) in DMAc (2 mL) was added anhydrous NaOAc (300 mg, 3.66 mmol) and stirred for 20 min at room temperature. After 20 min of stirring, 6-[2-cyano-3-[[ethyl(methyl)sulfamoyl]amino]-6-fluoro-phenoxy]-4-oxo-3-[(3R)-8-oxo-1-oxaspiro[4.5]decan-3-yl]quinazoline (A-16) (0.2 g, 351.12 mol) and AcOH (137.31 mol, 7.86 μL) were added and stirred for 3 h at room temperature. Subsequently, MP-CNBH₃ (500 mg, 1.37 mmol) was added and the resulting reaction mixture was stirred at room temperature for 16 hours. The reaction mixture was filtered through cotton pad and filtrate was concentrated under reduced pressure to get a crude residue which was purified by reverse phase column chromatography (Column: Redisep R_{f}Gold® reversed-phase, C18-Teledyne ISCO, 120 g, Mobile phase A: NH₄HCO₃ in milli-Q water; Mobile phase B: acetonitrile). Fractions were lyophilized to afford 140 mg of compound with 91% purity by UPLC-MS. Compound was re-purified by preparative HPLC [Column: X-Bridge C18 (150 mm*19 mm*5 m), Mobile phase A: 10 mm NH₄HCO₃ in milli-Q water; Mobile phase B: acetonitrile]. Fractions were collected and lyophilized to afford 6-[2-cyano-3-[[ethyl(methyl)sulfamoyl]amino]-6-fluoro-phenoxy]-3-[(3R)-8-[4-[4-[(2,6-dioxo-3-piperidyl)amino]-2-fluoro-phenyl]piperazin-1-yl]-1-oxaspiro[4.5]decan-3-yl]-4-oxo-quinazoline (Compound 40, 25 mg, 28.50 mol, 2% yield) as a white solid. UPLC-MS (ES+): m/z 860.2 [M+H]⁺. ¹H NMR (400 MHz, DMSO-d₆): δ 10.78 (d, J=3.00 Hz, 1H), 9.69 (bs, 1H), 8.33 (d, J=4.80 Hz, 1H), 7.77 (dd, J=1.60, 9.00 Hz, 1H), 7.677.55 (m, 2H), 7.36 (d, J=2.80 Hz, 2H), 6.86 (q, J=8.80 Hz, 1H), 6.53-6.42 (m, 2H), 5.90-5.85 (m, 1H), 5.31-5.21 (m, 1H), 4.30-4.20 (m, 1H), 4.17-4.05 (m, 2H), 3.20-2.75 (m, 11H), 2.70-2.60 (m, 4H), 2.47-2.40 (m, 1H), 2.11-1.51 (m, 9H), 1.50-1.30 (m, 2H), 1.04 (t, J=7.20 Hz, 3H).

### Synthesis D23: Synthesis of (3R)-3-[6-[2-cyano-3-[[ethyl(methyl)sulfamoyl]amino]-6-fluoro-phenoxy]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 41 and Compound 42)

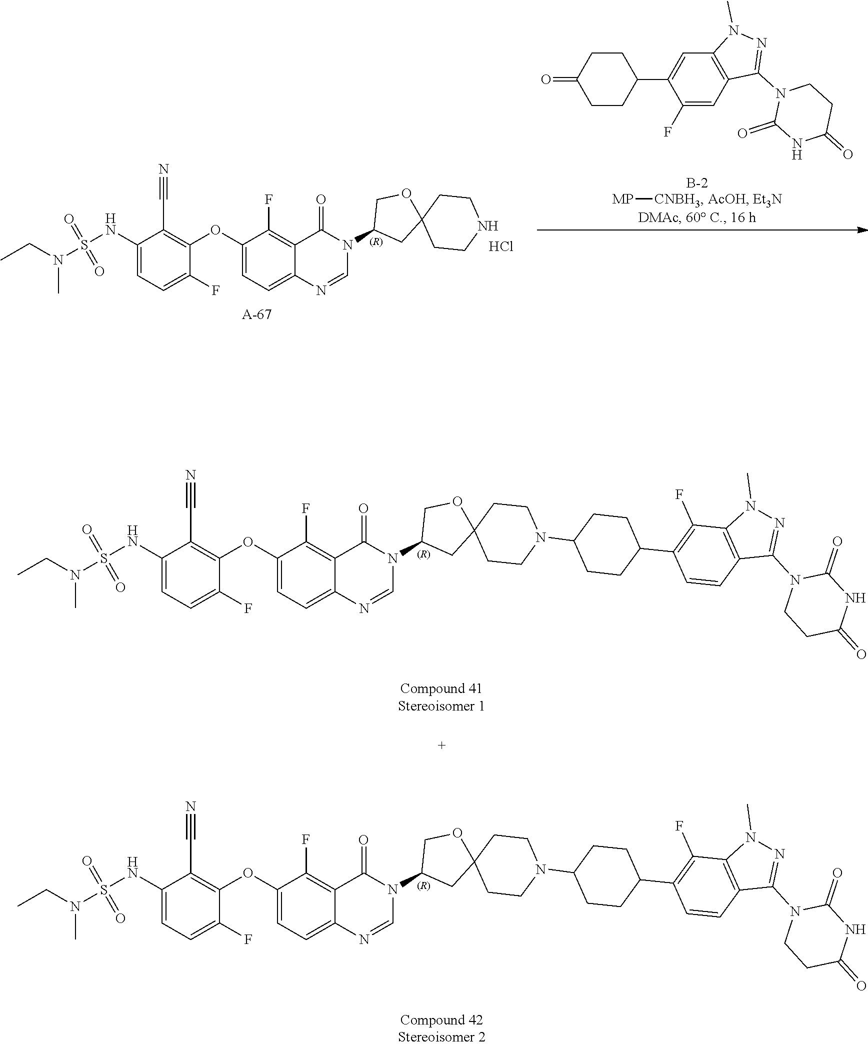

Into 20 mL pressure vial containing a well-stirred solution of (3R)-3-[6-[2-cyano-3-[[ethyl(methyl)sulfamoyl]amino]-6-fluoro-phenoxy]-5-fluoro-4-oxo-quinazolin-3-yl]-1-oxa-8-azaspiro[4.5]decane (A-67) (1 g, 1.64 mmol, HCl salt) in DMAc (4.67 mL) was added triethylamine (2.45 mmol, 342.14 μL) at 0° C. and the resulting mixture was stirred for 30 min at room temperature. After 30 min, 1-[5-fluoro-1-methyl-6-(4-oxocyclohexyl)indazol-3-yl]hexahydropyrimidine-2,4-dione (B-2) (586.47 mg, 1.64 mmol) and AcOH (4.91 mmol, 281.04 L) was added at 0° C. and stirred for 2 h at room temperature. Subsequently, MP-CNBH₃ (1.1 g, 1.64 mmol) was added at room temperature. Resulting reaction mixture was stirred at 60° C. for 16 hours. The reaction mixture was filtered through cotton pad and the filtrate was concentrated under reduced pressure to get a crude residue which was separated by preparative HPLC (Column: X-SELECT CSH (150 mm*19 mm*5 μm), Mobile phase A: 10 mM NH₄HCO₃ in milli-Q water; Mobile phase B: acetonitrile], flow: 15 mL/min). Both fractions from HPLC were collected separately and lyophilized to get two isomers. Further these two isomers were analyzed by SFC and assigned as stereoisomer 1 (Peak-1 (t_{R}=3.51 min) by SFC sequence, Peak-2 by Prep HPLC sequence) and stereoisomer 2 (Peak-2 (t_{R}=6.21 min) by SFC sequence, Peak-1 by Prep HPLC sequence).

Peak-1 (by SFC sequence): (3R)-3-[6-[2-cyano-3-[[ethyl(methyl)sulfamoyl]amino]-6-fluoro-phenoxy]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 41, 50 mg, 52.71 mol, 3% yield). UPLC-MS (ES+): m/z 917.5 [M+H]⁺. ¹H NMR (400 MHz, DMSO-d₆): δ 10.56 (s, 1H), 8.59 (bs, 1H), 8.32 (s, 1H), 7.48-7.26 (m, 6H), 5.35-5.25 (m, 1H), 4.25-4.10 (m, 2H), 4.03-4.00 (m, 3H), 3.91 (t, J=6.80 Hz, 2H), 3.65-3.42 (m, 1H), 3.25-3.09 (m, 2H), 3.08-2.97 (m, 2H), 2.76 (t, J=6.80 Hz, 2H), 2.61 (s, 3H), 2.45-2.38 (m, 2H), 2.25-1.40 (m, 15H), 1.03 (t, J=7.20 Hz, 3H).

Peak-2 (by SFC sequence): (3R)-3-[6-[2-cyano-3-[[ethyl(methyl)sulfamoyl]amino]-6-fluoro-phenoxy]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 42, 42.9 mg, 46.37 mol, 3% yield). UPLC-MS (ES+): m/z 917.5 [M+H]⁺. ¹H NMR (400 MHz, DMSO-d₆): δ 10.56 (s, 1H), 9.05 (bs, 1H), 8.32 (s, 1H), 7.55-7.25 (m, 6H), 5.38-5.28 (m, 1H), 4.24-4.10 (m, 2H), 4.00 (s, 3H), 3.91 (t, J=6.80 Hz, 2H), 3.60-3.37 (m, 2H), 3.22-3.06 (m, 1H), 3.05-2.85 (m, 4H), 2.76 (t, J=6.40 Hz, 2H), 2.63-2.56 (m, 4H), 2.27-1.76 (m, 10H), 1.75-1.58 (m, 4H), 1.03 (t, J=7.20 Hz, 3H).

### Synthesis D24: Synthesis of (3R)-3-[6-[2-cyano-3-[[ethyl(methyl)sulfamoyl]amino]-6-fluoro-phenoxy]-4-oxo-quinazolin-3-yl]-8-[4-[3-(2,4-dioxohexahydropyrimidin-1-yl)-5-fluoro-1-methyl-indazol-6-yl]-2-hydroxy-cyclohexyl]-1-oxa-8-azaspiro[4.5]decane (Compound 43), (3R)-3-[6-[2-cyano-3-[[ethyl(methyl)sulfamoyl]amino]-6-fluoro-phenoxy]-4-oxo-quinazolin-3-yl]-8-[4-[3-(2,4-dioxohexahydropyrimidin-1-yl)-5-fluoro-1-methyl-indazol-6-yl]-2-hydroxy-cyclohexyl]-1-oxa-8-azaspiro[4.5]decane (Compound 44) and (3R)-3-[6-[2-cyano-3-[[ethyl(methyl)sulfamoyl]amino]-6-fluoro-phenoxy]-4-oxo-quinazolin-3-yl]-8-[4-[3-(2,4-dioxohexahydropyrimidin-1-yl)-5-fluoro-1-methyl-indazol-6-yl]-2-hydroxy-cyclohexyl]-1-oxa-8-azaspiro[4.5]decane (Compound 45)

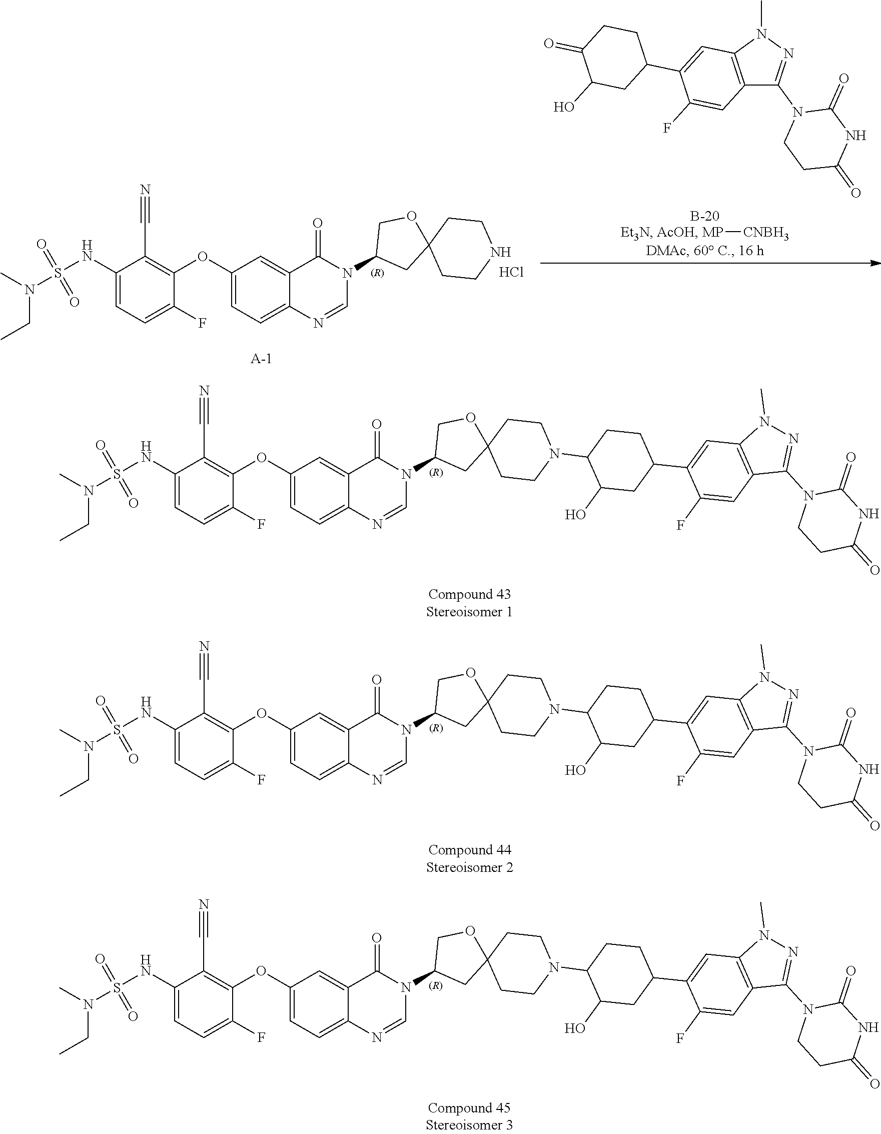

Into a 20 mL glass vial containing a well-stirred solution of (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1) (0.500 g, 843.07 μmol, HCl salt) and 1-[5-fluoro-6-[(1R)-3-hydroxy-4-oxo-cyclohexyl]-1-methyl-indazol-3-yl]hexahydropyrimidine-2,4-dione (B-20) (315.62 mg, 843.07 μmol) in DMAc (5 mL) was added triethylamine (2.53 mmol, 352.52 μL) and mixture was stirred for 1 hour at room temperature. To the above mixture, AcOH (843.07 μmol, 48.26 μL) was added and stirring was continued at room temperature for 2 hours. Subsequently, MIP-CNBH₃ (1.0 g, 843.07 μmol) was added, and the resulting reaction mixture was stirred at 60° C. for 16 hours. After the completion of the reaction, the reaction mixture was filtered and the filtrate was concentrated under reduced pressure to get a crude residue which was purified and separated by Prep HPLC (Column: X-Bridge C8 (250 mm*19 mm*5 μm), Mobile phase A: 10 mM NH₄HCO₃ in milli-Q water B: 100% acetonitrile). The first eluting fraction (Peak-1) at t_{R}=4.56 min was concentrated under reduced pressure at 40° C. and lyophilized to afford Compound 43 (assigned as stereoisomer 1).

Peak-1: (3R)-3-[6-[2-cyano-3-[[ethyl(methyl)sulfamoyl]amino]-6-fluoro-phenoxy]-4-oxo-quinazolin-3-yl]-8-[(1R,2S,4R)-4-[3-(2,4-dioxohexahydropyrimidin-1-yl)-5-fluoro-1-methyl-indazol-6-yl]-2-hydroxy-cyclohexyl]-1-oxa-8-azaspiro[4.5]decane (0.014 g, 14.52 mol, 2% yield) as a white solid. UPLC-MS (ES+): m/z 915.2 [M+H]⁺. ¹H NMR (400 MHz, DMSO-d₆): δ 10.56 (s, 1H), 8.97 (s, 1H), 8.31 (d, J=5.60 Hz, 1H), 7.76 (dd, J=3.60, 8.80 Hz, 1H), 7.66-7.63 (m, 1H), 7.57-7.50 (m, 1H), 7.42-7.33 (m, 4H), 5.69 (bs, 1H), 5.35-5.25 (m, 1H), 4.54-4.09 (m, 3H), 4.02-4.00 (m, 3H), 3.91 (t, J=6.80 Hz, 2H), 3.55-3.47 (m, 2H), 3.13-2.98 (m, 4H), 2.76 (t, J=6.40 Hz, 2H), 2.57 (bs, 3H), 2.41-2.34 (m, 1H), 2.22-1.62 (m, 12H), 1.05-1.01 (m, 3H).

The second eluting fraction (Peak-2) at t_{R}=4.67 min was concentrated under reduced pressure at 40° C. and lyophilized to afford Compound 44 (assigned as stereoisomer 2).

Peak-2: (3R)-3-[6-[2-cyano-3-[[ethyl(methyl)sulfamoyl]amino]-6-fluoro-phenoxy]-4-oxo-quinazolin-3-yl]-8-[(1R,2R,4R)-4-[3-(2,4-dioxohexahydropyrimidin-1-yl)-5-fluoro-1-methyl-indazol-6-yl]-2-hydroxy-cyclohexyl]-1-oxa-8-azaspiro[4.5]decane (0.009 g, 9.46 mol, 1% yield) as a white solid. UPLC-MS (ES+): m/z 915.2 [M+H]⁺. ¹H NMR (400 MHz, DMSO-d₆): δ 10.56 (s, 1H), 9.50 (bs, 1H), 8.33 (s, 1H), 7.77 (d, J=8.80 Hz, 1H), 7.66-7.64 (m, 1H), 7.58-7.31 (m, 5H), 5.30-5.28 (m, 1H), 4.23-4.02 (m, 3H), 4.00 (s, 3H), 3.91 (t, J=6.80 Hz, 2H), 3.23-3.17 (m, 2H), 3.04 (bs, 3H), 2.76 (t, J=6.40 Hz, 2H), 2.61 (s, 2H), 1.60-2.25 (m, 15H), 1.03 (t, J=7.20 Hz, 3H).

Similarly, the third eluting fraction at t_{R}=4.73 min was concentrated under reduced pressure at 40° C. and lyophilized to afford Compound 45 (assigned as stereoisomer 3).

Peak-3: (3R)-3-[6-[2-cyano-3-[[ethyl(methyl)sulfamoyl]amino]-6-fluoro-phenoxy]-4-oxo-quinazolin-3-yl]-8-[(1S,4R)-4-[3-(2,4-dioxohexahydropyrimidin-1-yl)-5-fluoro-1-methyl-indazol-6-yl]-2-hydroxy-cyclohexyl]-1-oxa-8-azaspiro[4.5]decane (0.010 g, 10.49 mol, 1% yield) as a white solid. UPLC-MS (ES+): m/z 915.6 [M+H]⁺. ¹H NMR (400 MHz, DMSO-d₆): δ 10.56 (s, 1H), 9.50 (bs, 1H), 8.32-8.30 (m, 1H), 7.77 (d, J=8.80 Hz, 1H), 7.67-7.34 (m, 6H), 5.30-5.28 (m, 1H), 4.21-4.06 (m, 2H), 4.01 (s, 3H), 3.91 (t, J=6.80 Hz, 2H), 3.58-3.43 (m, 2H), 3.38-2.80 (m, 6H), 2.76 (t, J=6.80 Hz, 2H), 2.63 (s, 3H), 1.35-2.30 (m, 11H), 1.04 (t, J=7.20 Hz, 3H).

### Synthesis D25: Synthesis of (3S)-3-[6-[2-cyano-3-[[ethyl(methyl)sulfamoyl]amino]-6-fluoro-phenoxy]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 46 and Compound 47)

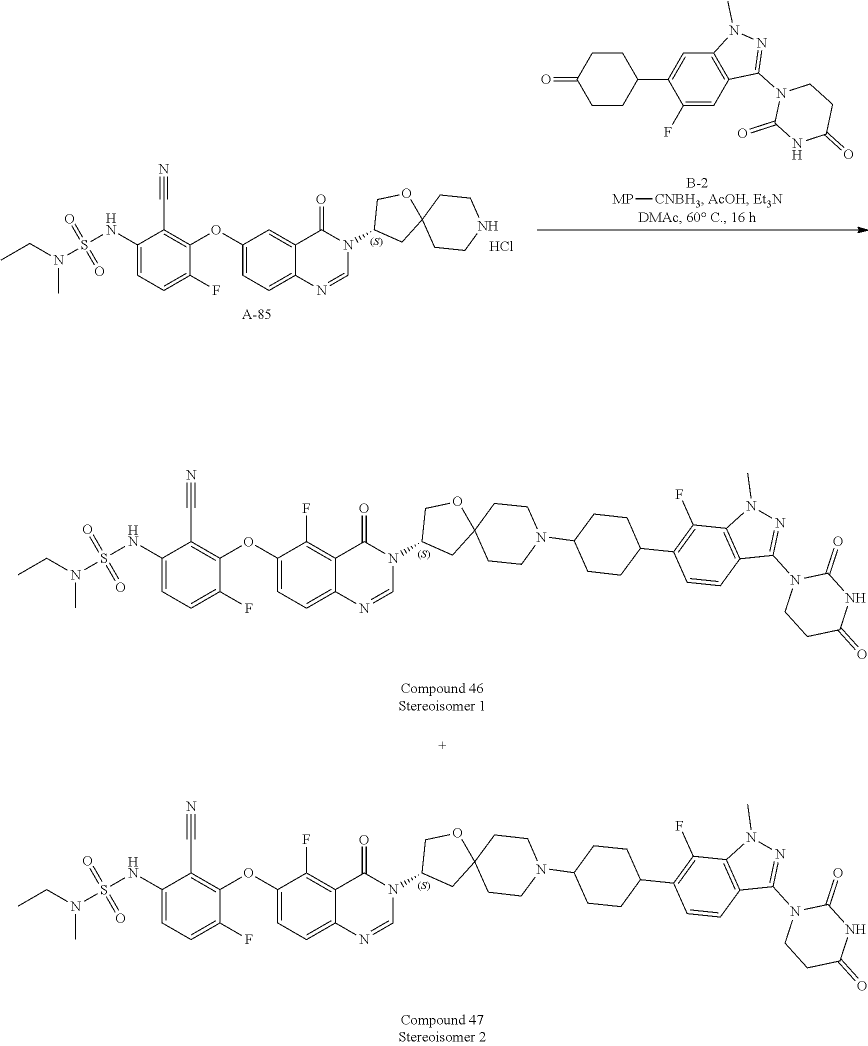

Into 20 mL pressure vial containing a well-stirred solution of 3-[(3S)-6-[2-cyano-3-[[ethyl(methyl)sulfamoyl]amino]-6-fluoro-phenoxy]-5-fluoro-4-oxo-quinazolin-3-yl]-1-oxa-8-azaspiro[4.5]decane (A-85) (943.69 mg, 1.54 mmol, HCl salt) in DMAc (10.04 mL) was added triethylamine (2.32 mmol, 322.88 μL) at 0° C. and the resulting mixture was stirred for 30 min at room temperature. After 30 min, 1-[5-fluoro-1-methyl-6-(4-oxocyclohexyl)indazol-3-yl]hexahydropyrimidine-2,4-dione (B-2) (553.44 mg, 1.54 mmol) and AcOH (4.63 mmol, 265.22 L) was added at 0° C. and stirred for 2 h at room temperature. Subsequently, MP-CNBH₃ (1.1 g, 1.54 mmol) was added at room temperature. Resulting reaction mixture was stirred at 60° C. for 16 hours. The reaction mixture was filtered through a cotton pad and the filtrate was concentrated under reduced pressure to get a crude residue which was separated by preparative HPLC [Column: X-SELECT CSH (150 mm*19 mm*5 μm), Mobile phase A: 10 mM NH₄HCO₃ in milli-Q water; Mobile phase B: acetonitrile], flow: 15 mL/min]. Both fractions from HPLC were collected separately and lyophilized to get two isomers. Further, these two isomers were analyzed by SFC and assigned as stereoisomer 1 (Peak-1 (t_{R}=2.97 min) by SFC sequence, Peak-2 by Prep HPLC sequence) and stereoisomer 2 (Peak-2 (t_{R}=4.03 min) by SFC sequence, Peak-1 by Prep HPLC sequence).

Peak-1 (by SFC sequence): (3S)-3-[6-[2-cyano-3-[[ethyl(methyl)sulfamoyl]amino]-6-fluoro-phenoxy]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 46, 81.76 mg, 87.34 mol, 6% yield). UPLC-MS (ES+): m/z 917.5 [M+H]⁺. ¹H NMR (400 MHz, DMSO-d₆): δ 10.56 (s, 1H), 8.64 (bs, 1H), 8.32 (s, 1H), 7.49-7.28 (m, 6H), 5.36-5.28 (m, 1H), 4.22-4.10 (m, 2H), 4.03 (s, 3H), 3.91 (t, J=6.80 Hz, 2H), 3.75-3.40 (m, 2H), 3.25-3.09 (m, 4H), 2.76 (t, J=6.40 Hz, 3H), 2.63 (s, 3H), 2.50-2.38 (m, 3H), 2.28-1.60 (m, 12H), 1.03 (t, J=6.80 Hz, 3H).

Peak-2 (by SFC sequence): (3S)-3-[6-[2-cyano-3-[[ethyl(methyl)sulfamoyl]amino]-6-fluoro-phenoxy]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 47, 87.27 mg, 91.43 mol, 6% yield). UPLC-MS (ES+): m/z 917.5 [M+H]⁺. ¹H NMR (400 MHz, DMSO-d₆): δ 10.56 (s, 1H), 9.41 (bs, 1H), 8.32 (s, 1H), 7.54 (d, J=6.00 Hz, 1H), 7.51-7.35 (m, 4H), 7.25-7.22 (m, 1H), 5.35-5.29 (m, 1H), 4.24-4.10 (m, 2H), 4.00 (s, 3H), 3.90 (t, J=6.80 Hz, 2H), 3.23-3.08 (m, 3H), 3.05-2.97 (m, 2H), 2.88-2.95 (m, 1H), 2.87-2.81 (m, 3H), 2.76 (t, J=6.40 Hz, 2H), 2.64-2.58 (m, 3H), 2.47-2.34 (m, 1H), 2.22-2.09 (m, 3H), 2.08-1.80 (m, 5H), 1.84-1.75 (m, 4H), 1.03 (t, J=7.20 Hz, 3H).

### Synthesis D26: Synthesis of (3S)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 48 and Compound 49)

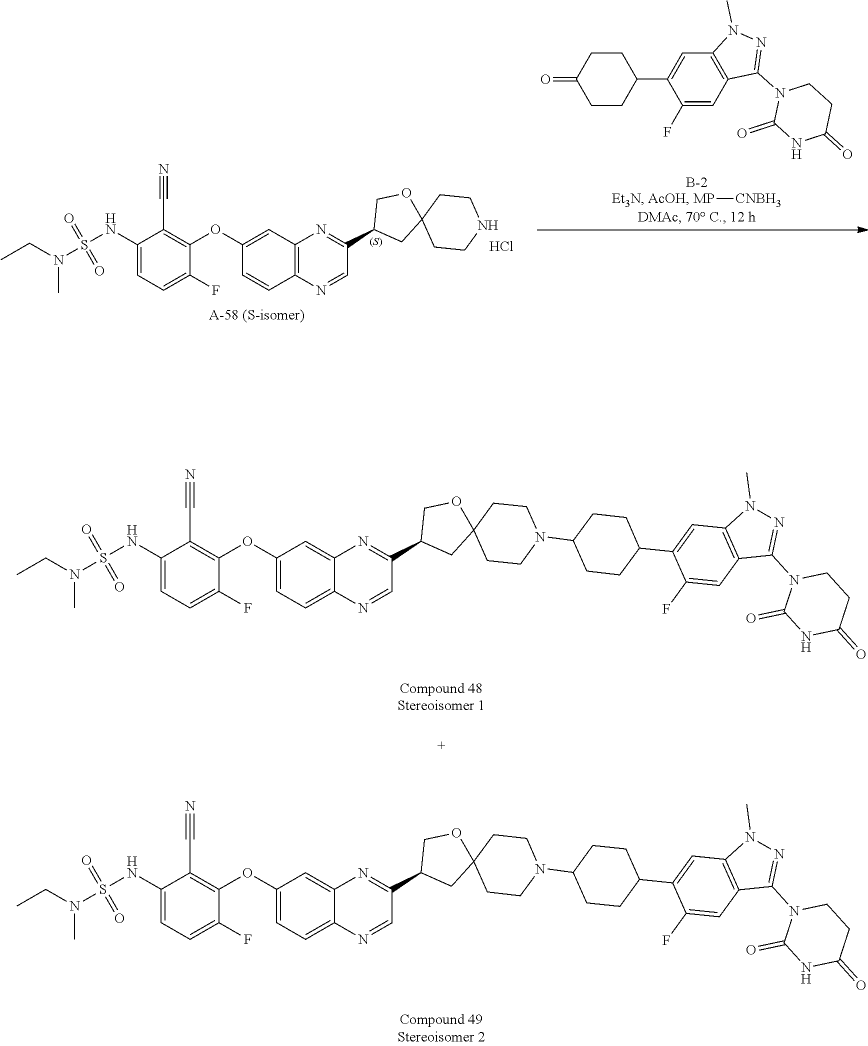

Into a 10 mL pressure vial containing a well-stirred solution of (3S)-3-[7-[2-cyano-3-[[ethyl(methyl)sulfamoyl]amino]-6-fluoro-phenoxy]quinoxalin-2-yl]-1-oxa-8-azaspiro[4.5]decane (A-58) (170 mg, 279.13 mol, HCl salt) in DMAc (5.01 mL) was added triethylamine (837.38 mol, 116.71 μL) at room temperature. Reaction mixture was stirred at room temperature for 5 min. To the above mixture, AcOH (558.25 mol, 31.96 μL) and 1-[5-fluoro-1-methyl-6-(4-oxocyclohexyl)indazol-3-yl]hexahydropyrimidine-2,4-dione (B-2) (129.81 mg, 334.95 mol) were added and stirred for 2 h at room temperature. Subsequently, MP-CNBH₃ (340 mg, 279.13 mol) was added at room temperature and resulting reaction mixture was stirred at 70° C. for 12 hours. After completion of the reaction, the reaction mixture was filtered through cotton and filtrate was concentrated under reduced pressure to get a crude residue which was purified by prep HPLC [Column: YMC Actus c18 (250 mm*20 mm*5 μm) and [Mobile phase A:10 mm NH₄HCO₃ in milli-Q water; Mobile phase B: acetonitrile]. Both fractions from HPLC were lyophilized to get two isomers. Further these two isomers were analyzed by SFC and assigned as stereoisomer 1 (Peak-1 (t_{R}=4.6 min) by SFC sequence, Peak-2 by Prep HPLC sequence) and stereoisomer 2 (Peak-2 (t_{R}=7.81 min) by SFC sequence, Peak-1 by Prep HPLC sequence).

Peak-1 (by SFC sequence): (3S)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 48, 25 mg, 28.03 mol, 10% yield) as an off-white solid. Assigned as stereoisomer 1 (Chiral SFC t_{R}=4.6 min). UPLC-MS (ES+): m/z 883.3 [M+H]⁺. ¹H NMR (400 MHz, DMSO-d₆): δ 10.563 (s, 1H), 8.86 (s, 1H), 8.62 (bs, 1H), 8.14 (d, J=9.2 Hz, 1H), 7.71 (dd, J=2.8, 9.2 Hz, 1H), 7.50-7.32 (m, 4H), 7.10 (s, 1H), 4.29 (bs, 1H), 4.03 (s, 3H), 3.99-3.90 (m, 4H), 3.53 (bs, 1H), 3.38-3.01 (m, 5H), 2.76 (t, J=6.4 Hz, 2H), 2.68-2.50 (m, 3H), 2.34-1.70 (m, 15H), 1.04 (t, J=7.20 Hz, 3H).

Peak-2 (by SFC sequence): (3S)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 49, 10 mg, 10.82 mol, 4% yield) as an off-white solid. Assigned as stereoisomer 2 (Chiral SFC t_{R}=7.81 min). UPLC-MS (ES+): m/z 883.3 [M+H]⁺. ¹H NMR (400 MHz, DMSO-d₆): δ 10.87 (bs, 1H), 10.56 (s, 1H), 8.86 (s, 1H), 8.14-8.12 (m, 1H), 7.70 (dd, J=2.8, 9.2 Hz, 1H), 7.55 (d, J=6.0 Hz, 1H), 7.40-7.26 (m, 3H), 7.08 (s, 1H), 4.29 (bs, 1H), 4.02-3.99 (m, 5H), 3.90 (t, J=6.40 Hz, 2H), 3.33-2.96 (m, 7H), 2.76 (t, J=6.40 Hz, 2H), 2.68-2.68 (m, 3H), 2.33-1.68 (m, 15H), 1.0 (t, J=7.2 Hz, 3H).

### Synthesis D27: Synthesis of (3R)-3-[6-[2-cyano-3-[[ethyl(methyl)sulfamoyl]amino]-6-fluoro-phenoxy]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 50 and Compound 51)

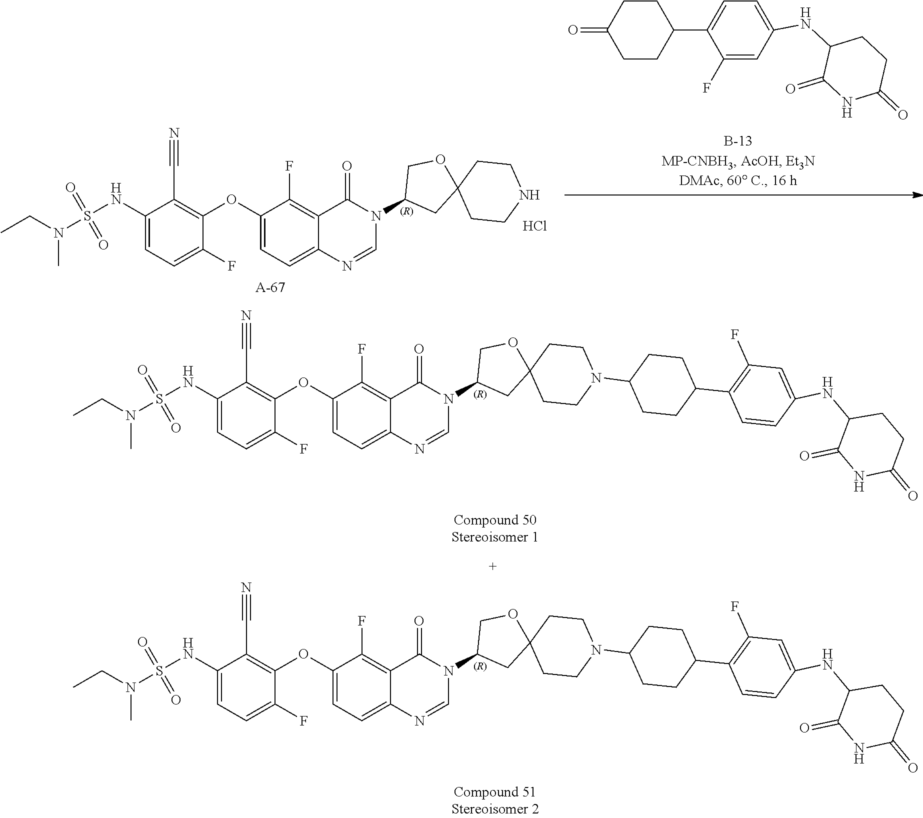

Into a 20 mL pressure vial containing a well-stirred solution of (3R)-3-[6-[2-cyano-3-[[ethyl(methyl)sulfamoyl]amino]-6-fluoro-phenoxy]-5-fluoro-4-oxo-quinazolin-3-yl]-1-oxa-8-azaspiro[4.5]decane (A-67) (1.00 g, 1.64 mmol, HCl salt) in DMAc (4.67 mL) were added triethylamine (2.45 mmol, 342.14 μL) and 3-[3-fluoro-4-(4-oxocyclohexyl)anilino]piperidine-2,6-dione (B-13) (520.97 mg, 1.64 mmol) at 0° C. and the resulting mixture was stirred for 30 min at room temperature. After 30 min, AcOH (4.91 mmol, 281.04 μL) was added at 0° C. and stirred for 2 h at room temperature. Subsequently, MP-CNBH₃ (1.1 g, 1.64 mmol) was added at room temperature. Resulting reaction mixture was stirred at 60° C. for 16 hours. The reaction mixture was filtered through cotton pad and the filtrate was concentrated under reduced pressure to get a crude residue which was purified by preparative HPLC (Column: Redisep Rf Gold® reversed-phase C18-teledyne ISCO, 120 g), [Mobile phase A: 10 mM NH₄OH (pH-10); Mobile phase B: acetonitrile, flow rate: 14 mL/min]. Both fractions from HPLC were collected separately and lyophilized to get two isomers. Further these two isomers were analyzed by SFC and assigned as stereoisomer 1 (Peak-1 (t_{R}=2.91 min) by SFC sequence, Peak-2 by Prep HPLC sequence) and stereoisomer 2 (Peak-2 (t_{R}=3.47 min) by SFC sequence, Peak-1 by Prep HPLC sequence).

Peak-1 (by SFC sequence): (3R)-3-[6-[2-cyano-3-[[ethyl(methyl)sulfamoyl]amino]-6-fluoro-phenoxy]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 50, 27 mg, 30.67 mol, 2% yield) as an off-white solid. UPLC-MS (ES+): m/z 877.2 [M+H]⁺. ¹H NMR (400 MHz, DMSO-d₆): δ 10.79 (s, 1H), 8.31 (s, 1H), 7.50-7.40 (m, 2H), 7.28-6.80 (m, 4H), 6.51-6.40 (m, 2H), 6.08-5.92 (m, 1H), 5.33-5.28 (m, 1H), 4.35-4.25 (m, 1H), 4.24-4.06 (m, 2H), 3.61-3.40 (m, 2H), 3.20-2.82 (m, 5H), 2.78-2.68 (m, 2H), 2.65-2.57 (m, 4H), 2.45-2.36 (m, 2H), 2.30-1.60 (m, 14H), 1.03 (t, J=7.20 Hz, 3H).

Peak-2 (by SFC sequence): (3R)-3-[6-[2-cyano-3-[[ethyl(methyl)sulfamoyl]amino]-6-fluoro-phenoxy]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 51, 30 mg, 33.87 mol, 2% yield) as an off-white solid. UPLC-MS (ES+): m/z 877.2 [M+H]⁺. ¹H NMR (400 MHz, DMSO-d₆): δ 10.79 (s, 1H), 9.00 (bs, 1H), 8.30 (s, 1H), 7.49-7.42 (m, 2H), 7.39-7.18 (m, 2H), 7.00-6.95 (m, 1H), 6.46-6.43 (m, 2H), 6.03-6.01 (m, 1H), 5.35-5.25 (m, 1H), 4.33-4.27 (m, 1H), 4.25-4.10 (m, 2H), 3.05-3.25 (m, 2H), 3.04-2.95 (m, 2H), 2.79-2.69 (m, 1H), 2.55-2.65 (m, 6H), 2.46-2.36 (m, 2H), 2.25-1.70 (m, 12H), 1.69-1.43 (m, 4H), 1.02 (t, J=7.20 Hz, 3H).

### Synthesis D28: Synthesis of (3S)-3-[6-[2-cyano-3-[[ethyl(methyl)sulfamoyl]amino]-6-fluoro-phenoxy]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 52 and Compound 53)

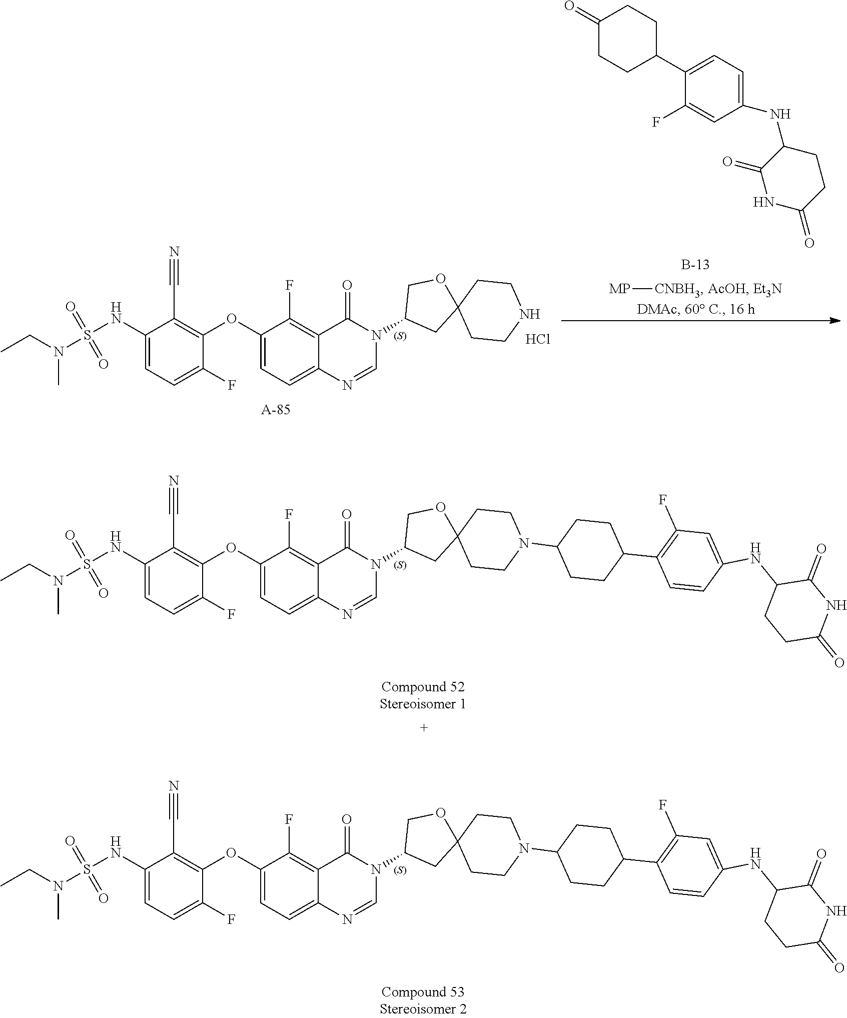

Into 20 mL pressure vial containing a well stirred solution of (3S)-3-[6-[2-cyano-3-[[ethyl(methyl)sulfamoyl]amino]-6-fluoro-phenoxy]-5-fluoro-4-oxo-quinazolin-3-yl]-1-oxa-8-azaspiro[4.5]decane (A-85) (700.00 mg, 1.15 mmol, HCl salt) in DMAc (5 mL) was added triethylamine (1.72 mmol, 239.50 μL) at 0° C. and stirred for 30 min at room temperature. After 30 min, 3-[3-fluoro-4-(4-oxocyclohexyl)anilino]piperidine-2,6-dione (B-13) (364.68 mg, 1.15 mmol) and AcOH (3.44 mmol, 196.73 μL) were added at 0° C. and stirred for 2 hours at room temperature. Subsequently, MP-CNBH₃ (770 mg, 1.15 mmol) was added at room temperature. And resulting reaction mixture was stirred at 60° C. for 16 hours. The reaction mixture was filtered through cotton pad and filtrate was concentrated under reduced pressure to get a crude residue which was separated by preparative HPLC [Column: X-SELECT CSH (150 mm*19 mm*5 μm), Mobile phase A: 10 mM NH₄HCO₃ in milli-Q water; Mobile phase B: acetonitrile, flow: 15 mL/min]. Both fractions from HPLC were collected separately and lyophilized to get two isomers. Further these two isomers were analyzed by SFC and assigned as stereoisomer 1 (Peak-1 (t_{R}=2.87 min) by SFC sequence, Peak-2 by Prep HPLC sequence) and stereoisomer 2 (Peak-2 (t_{R}=3.39 min) by SFC sequence, Peak-1 by Prep HPLC sequence).

Peak-1 (by SFC sequence): (3S)-3-[6-[2-cyano-3-[[ethyl(methyl)sulfamoyl]amino]-6-fluoro-phenoxy]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 52, 31 mg, 34.19 mol, 3% yield) UPLC-MS (ES+): m/z 877.2 [M+H]⁺. ¹H NMR (400 MHz, DMSO-d₆): δ 10.80 (s, 1H), 8.50 (bs, 1H), 8.31 (s, 1H), 7.50-7.40 (m, 2H), 7.39-6.95 (m, 3H), 6.53-6.40 (m, 2H), 6.10-5.95 (m, 1H), 5.35-5.25 (m, 1H), 4.35-4.27 (m, 1H), 4.26-4.10 (m, 2H), 3.65-3.48 (m, 1H), 3.21-3.08 (m, 2H), 3.05-2.90 (m, 3H), 2.88-2.70 (m, 2H), 2.65-2.55 (m, 2H), 2.48-2.38 (m, 3H), 2.25-1.60 (m, 14H), 1.58-1.30 (m, 2H), 1.03 (t, J=7.20 Hz, 3H).

Peak-2 (by SFC sequence): (3S)-3-[6-[2-cyano-3-[[ethyl(methyl)sulfamoyl]amino]-6-fluoro-phenoxy]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 53, 28 mg, 30.90 mol, 3% yield). UPLC-MS (ES+): m/z 917.5 [M+H]⁺. ¹H NMR (400 MHz, DMSO-d₆): δ 10.80 (s, 1H), 9.00 (bs, 1H), 8.30 (s, 1H), 7.49-7.22 (m, 4H), 6.97 (t, J=8.40 Hz, 1H), 6.46-6.42 (m, 2H), 6.02 (d, J=7.60 Hz, 1H), 5.35-5.25 (m, 1H), 4.35-4.27 (m, 1H), 4.26-4.10 (m, 2H), 3.21-2.70 (m, 8H), 2.65-2.55 (m, 6H), 2.25-1.70 (m, 12H), 1.58-1.40 (m, 3H), 1.02 (t, J=7.20 Hz, 3H).

### Synthesis D29: Synthesis of (3R)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 54 and Compound 55)

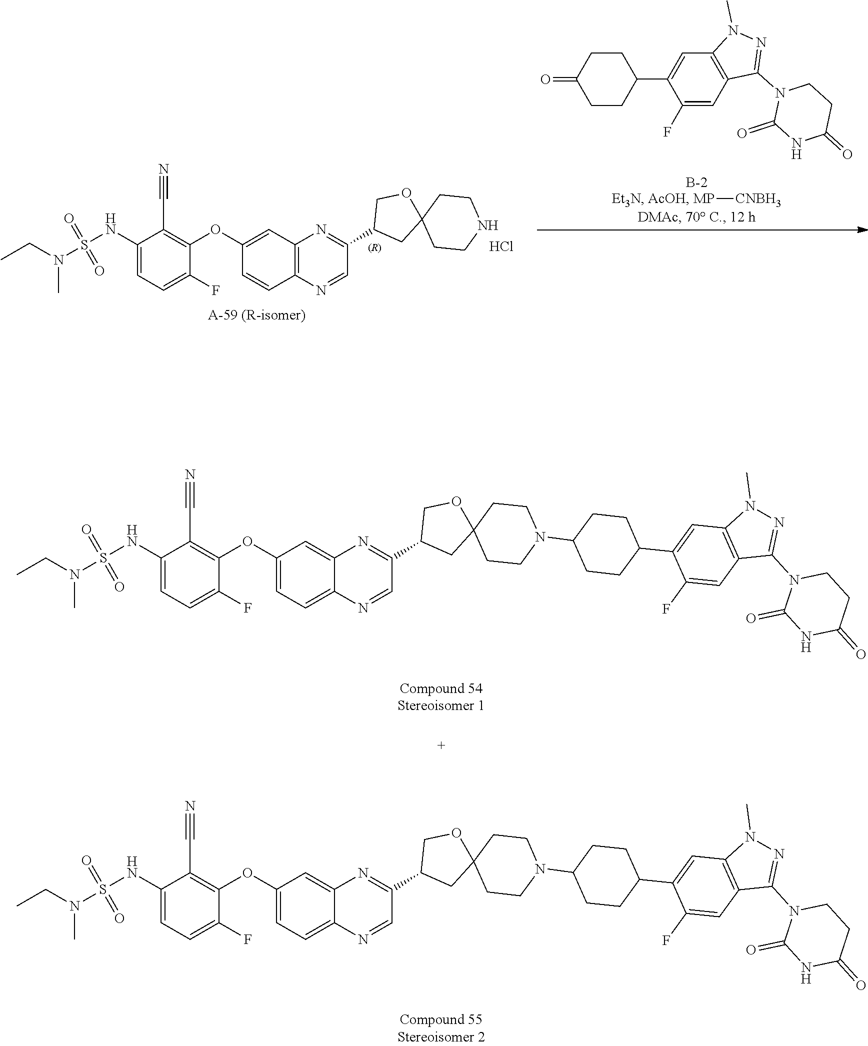

Into a 10 mL pressure vial containing a well-stirred solution of (3R)-3-[7-[2-cyano-3-[[ethyl(methyl)sulfamoyl]amino]-6-fluoro-phenoxy]quinoxalin-2-yl]-1-oxa-8-azaspiro[4.5]decane (A-59) (140 mg, 208.88 mol, HCl salt) in DMAc (5 mL) was added triethylamine (626.65 mol, 87.34 μL) and mixture was stirred at room temperature for 5 min. To the above mixture, AcOH (417.77 mol, 23.92 μL) and 1-[5-fluoro-1-methyl-6-(4-oxocyclohexyl)indazol-3-yl]hexahydropyrimidine-2,4-dione (B-2) (97.14 mg, 250.66 mol) were added and stirring was continued for another 2 hours at room temperature. Subsequently, MP-CNBH₃ (280 mg, 208.88 mol) was added at room temperature and resulting reaction mixture was stirred at 70° C. for 12 hours. After completion of the reaction, the reaction mixture was filtered through cotton and filtrate was concentrated under reduced pressure to get a crude residue which was purified by preparative HPLC [Column: YMC Actus C18 (250 mm*20 mm*5 μm), Mobile phase A: 10 mM NH₄HCO₃ in milli-Q water; Mobile phase B: acetonitrile]. Both fractions from HPLC were lyophilized to get two isomers. Further these two isomers were analyzed by SFC and assigned as stereoisomer 1(Peak-1 (t_{R}=8.13 min) by SFC sequence, Peak-2 by Prep HPLC sequence) and stereoisomer 2 (Peak-2 (t_{R}=14.55 min) by SFC sequence, Peak-1 by Prep HPLC sequence).

Peak-1 (by SFC sequence): (3R)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 54, 5 mg, 5.52 mol, 3% yield) as an off-white solid. Peak-1 was assigned as stereoisomer 1 (Chiral SFC t_{R}=8.13 min). UPLC-MS (ES+): m/z 883.1 [M+H]⁺. ¹H NMR (400 MHz, DMSO-d₆): δ 10.56 (s, 1H), 8.86 (s, 1H), 8.69 (bs, 1H), 8.14 (d, J=9.2 Hz, 1H), 7.69 (d, J=9.2 Hz, 1H), 7.50-7.29 (m, 3H), 7.09 (s, 1H), 4.28 (bs, 1H), 4.03 (s, 3H), 3.99-3.90 (m, 4H), 3.59 (s, 1H), 3.25-2.99 (m, 5H), 2.76 (t, J=6.80 Hz, 2H), 2.68-2.67 (m, 4H), 2.23-1.56 (m, 15H), 1.03 (t, J=9.6 Hz, 3H).

Peak-2 (by SFC sequence): (3R)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 55, 20 mg, 21.82 mol, 10% yield) as an off-white solid. Peak-2 was assigned as stereoisomer 2 (chiral SFC t_{R}=14.55 min). UPLC-MS (ES+): m/z 883.1 [M+H]⁺. ¹H NMR (400 MHz, DMSO-d₆): δ 10.56 (s, 1H), 9.20 (bs, 1H), 8.89 (s, 1H), 8.14 (dd, J=9.0, 5.2 Hz, 1H), 7.71 (dd, J=2.8, 9.2 Hz, 1H), 7.55-7.54 (m, 4H), 7.09 (s, 1H), 4.30 (bs, 1H), 4.03-3.97 (m, 5H), 3.90 (t, J=7.2, 2H), 3.25-2.90 (m, 7H), 2.76 (t, J=6.80 Hz, 2H), 2.68-2.62 (m, 3H), 2.33-1.68 (m, 15H), 1.04 (t, J=7.2 Hz, 3H).

### Synthesis D30: Synthesis of (3R)-3-[6-[2-cyano-3-[[ethyl(methyl)sulfamoyl]amino]-6-fluoro-anilino]-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 56 and Compound 57)

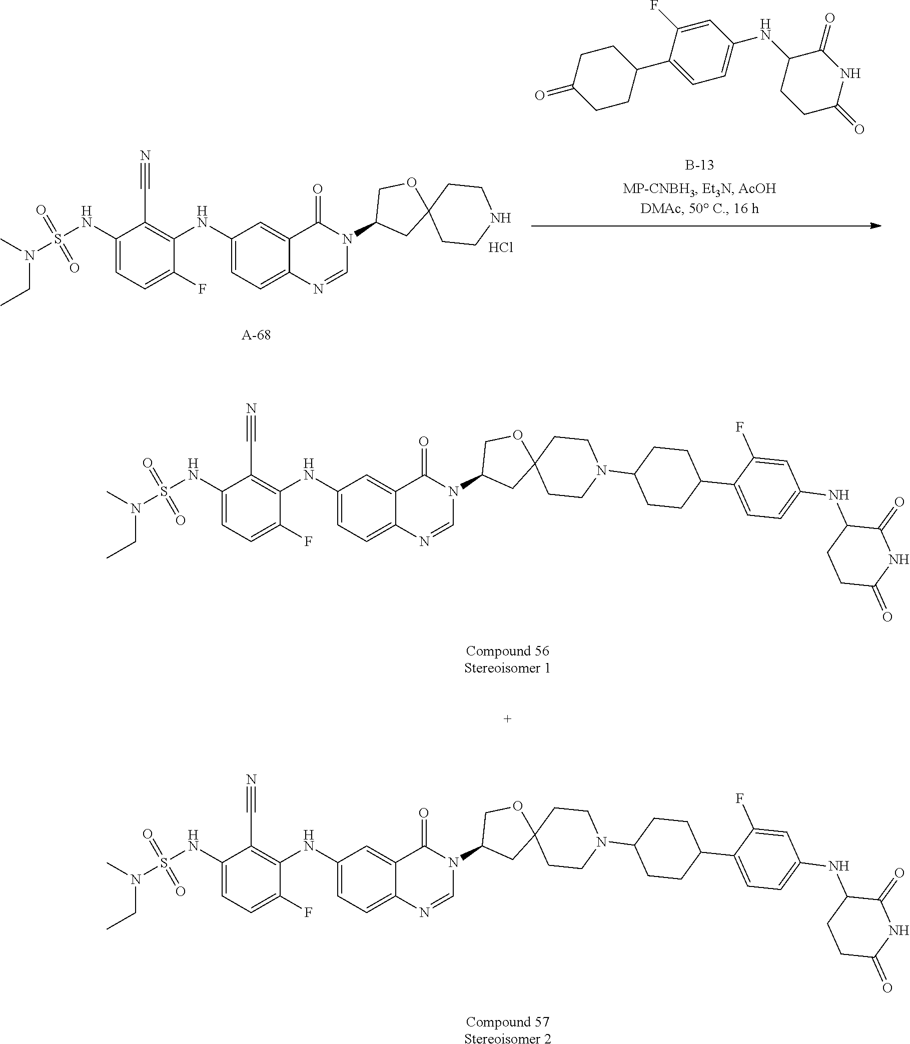

Into a 20 mL pressure vial containing a well-stirred solution of (3R)-3-[6-[2-cyano-3-[[ethyl(methyl)sulfamoyl]amino]-6-fluoro-anilino]-4-oxo-quinazolin-3-yl]-1-oxa-8-azaspiro[4.5]decane (A-68) (300 mg, 506.68 mol, HCl salt) in DMAc (5 mL) was added triethylamine (1.52 mmol, 211.87 μL) at room temperature under nitrogen atmosphere and mixture was stirred for 30 min. Reaction mixture was cooled to 0° C. and was added 3-[3-fluoro-4-(4-oxocyclohexyl)anilino]piperidine-2,6-dione (B-13) (193.56 mg, 608.02 mol) followed by AcOH (760.03 mol, 43.46 μL) and resulting mixture was stirred for 1 h at room temperature. Finally, MP-CNBH₃ (500 mg, 506.68 mol) was added, and the reaction mixture was stirred at 50° C. for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fractions were lyophilized to afford 150 mg of mixture of cis and trans isomers. Further cis and trans isomers were separated by preparative HPLC [Column: YMC ACTUS C18 (250 mm*19 mm*5 m), Mobile phase A: 10 mM NH₄HCO₃ in milli-Q water; Mobile phase B: acetonitrile]. The first eluting fraction (Peak-1) was concentrated under reduced pressure at 40° C. and lyophilized to afford Compound 56 (assigned as stereoisomer 1).

Peak-1: (3R)-3-[6-[2-cyano-3-[[ethyl(methyl)sulfamoyl]amino]-6-fluoro-anilino]-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 56, 18.74 mg, 20.31 mol, 4% yield, formic acid salt) as an off-white solid. UPLC-MS (ES+): m/z 858.0 [M+H]⁺. ¹H NMR (400 MHz, DMSO-d₆): δ 10.79 (s, 1H), 8.62 (bs, 1H), 8.18 (s, 1H), 7.55 (d, J=8.80 Hz, 1H), 7.48-7.20 (m, 4H), 7.05-6.92 (m, 1H), 6.51-6.42 (m, 2H), 6.01 (d, J=7.60 Hz, 1H), 5.40-5.30 (m, 1H), 4.40-4.27 (m, 1H), 4.19-4.11 (m, 2H), 3.13-2.88 (m, 6H), 2.86-2.70 (m, 3H), 2.64-2.59 (m, 4H), 2.44-2.39 (m, 1H), 2.20-1.79 (m, 11H), 1.66-1.43 (m, 4H), 1.03 (t, J=7.20 Hz, 3H).

Similarly, the second eluted fraction (Peak-2) was concentrated under reduced pressure at 40° C. and lyophilized to afford Compound 57 (assigned as stereoisomer 2).

Peak-2: (3R)-3-[6-[2-cyano-3-[[ethyl(methyl)sulfamoyl]amino]-6-fluoro-anilino]-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 57, 24.86 mg, 26.77 mol, 5% yield, formic acid salt) as an off-white solid. UPLC-MS (ES+): m/z 858.2 [M+H]⁺. ¹H NMR (400 MHz, DMSO-d₆): δ 10.79 (s, 1H), 8.74 (s, 1H), 8.20 (s, 1H), 8.14 (s, 1H), 7.57 (d, J=8.80 Hz, 1H), 7.45 (bs, 1H), 7.32 (d, J=8.80 Hz, 1H), 7.28 (s, 1H), 7.23 (dd, J=4.00, 9.20 Hz, 1H), 7.03 (t, J=8.80 Hz, 1H), 6.48-6.42 (m, 2H), 5.99 (d, J=7.60 Hz, 1H), 5.32 (s, 1H), 4.33-4.27 (m, 1H), 4.12-4.10 (m, 2H), 3.10-3.06 (m, 2H), 2.78-2.60 (m, 8H), 2.59-2.56 (m, 1H), 2.49-2.44 (m, 2H), 2.42-2.36 (m, 1H), 2.07-1.81 (m, 11H), 1.62-1.53 (m, 4H), 1.05 (t, J=7.20 Hz, 3H).

### Synthesis D31: Synthesis of (3S)-3-[7-[2-cyano-3-[[ethyl(methyl)sulfamoyl]amino]-6-fluoro-phenoxy]quinoxalin-2-yl]-8-[4-[4-[(2,6-dioxo-3-piperidyl)amino]-2-fluoro-phenyl]cyclohexyl]-1-oxa-8-azaspiro[4.5]decane (Compound 58 and Compound 59)

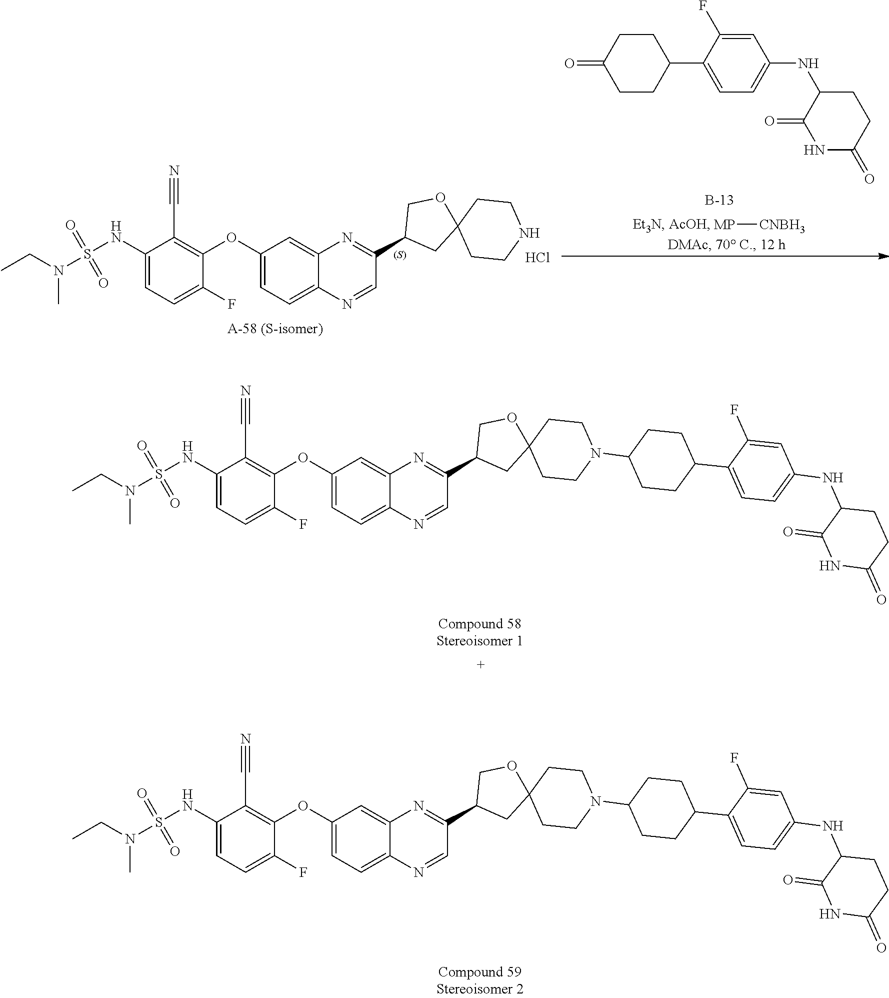

Into a 20 mL pressure vial containing a well-stirred solution of (3S)-3-[7-[2-cyano-3-[[ethyl(methyl)sulfamoyl]amino]-6-fluoro-phenoxy]quinoxalin-2-yl]-1-oxa-8-azaspiro[4.5]decane (A-58) (350 mg, 606.51 mol, HCl salt) in DMAc (5 mL) and was added triethylamine (1.82 mmol, 253.61 μL) at room temperature and mixture was stirred at room temperature for 5 minutes. To the above mixture, AcOH (1.21 mmol, 69.44 μL) and 3-[3-fluoro-4-(4-oxocyclohexyl)anilino]piperidine-2,6-dione (B-13) (231.69 mg, 727.82 mol) were added and mixture was continued to stir for 2 hours at room temperature. Subsequently, MP-CNBH₃ (700 mg, 606.51 mol) was added at room temperature and resulting reaction mixture was stirred at 70° C. for 12 hours. After completion of the reaction, the reaction mixture was filtered through cotton and filtrate was concentrated under reduced pressure to get a crude residue which was purified by preparative HPLC [Column: SHIMPACK C18 (150 mm*19 mm*5 μm), Mobile phase A: 10 mM ammonium bicarbonate in milli-Q water; Mobile phase B: acetonitrile]. Both fractions from HPLC were lyophilized to get two isomers. Further these two isomers were analyzed by SFC and assigned as stereoisomer 1 (Peak-1 (t_{R}=2.01 min) by SFC sequence, Peak-2 by Prep HPLC sequence) and stereoisomer 2 (Peak-2 (t_{R}=3.14 min) by SFC sequence, Peak-1 by Prep HPLC sequence).

Peak-1 (by SFC sequence): (3S)-3-[7-[2-cyano-3-[[ethyl(methyl)sulfamoyl]amino]-6-fluoro-phenoxy]quinoxalin-2-yl]-8-[4-[4-[(2,6-dioxo-3-piperidyl)amino]-2-fluoro-phenyl]cyclohexyl]-1-oxa-8-azaspiro[4.5]decane (Compound 58, 58 mg, 68.08 mol, 11% yield) as a white solid. Peak-1 was assigned as stereoisomer 1 (Chiral SFC t_{R}=2.01 min). UPLC-MS (ES+): m/z 843.4 [M+H]⁺. ¹H NMR (400 MHz, DMSO-d₆): δ 10.79 (s, 1H), 8.86 (s, 1H), 8.48 (bs, 1H), 8.14 (d, J=9.2 Hz, 1H), 7.70 (dd, J=2.8, 9.2 Hz, 1H), 7.44-7.40 (m, 1H), 7.33-7.25 (m, 1H), 7.15-7.10 (m, 2H), 6.50-6.43 (m, 2H), 6.03 (d, J=7.6 Hz, 1H), 4.33-4.29 (m, 2H), 4.20-3.99 (m, 2H), 3.60-3.50 (m, 1H), 3.20-2.80 (m, 5H), 2.78-2.60 (m, 6H), 2.30-2.06 (m, 2H), 2.02-1.54 (m, 13H), 1.03 (t, J=7.2 Hz, 3H).

Peak-2 (by SFC sequence): (3S)-3-[7-[2-cyano-3-[[ethyl(methyl)sulfamoyl]amino]-6-fluoro-phenoxy]quinoxalin-2-yl]-8-[4-[4-[(2,6-dioxo-3-piperidyl)amino]-2-fluoro-phenyl]cyclohexyl]-1-oxa-8-azaspiro[4.5]decane (Compound 59, 30 mg, 34.52 mol, 6% yield) as a white solid. Peak-2 was assigned as stereoisomer 2 (Chiral SFC t_{R}=3.14 min). UPLC-MS (ES+): m/z 843.4 [M+H]⁺. ¹H NMR (400 MHz, DMSO-d₆): δ 10.792 (s, 1H), 9.06 (bs, 1H), 8.86 (s, 1H), 8.14 (d, J=9.2 Hz, 1H), 7.70 (dd, J=2.8, 9.2 Hz, 1H), 7.45-6.90 (m, 4H), 6.49-6.43 (m, 2H), 6.04-5.99 (m, 1H), 4.32-4.8 (m, 2H), 4.10-3.98 (m, 2H), 3.65-3.51 (m, 1H), 3.20-2.81 (m, 6H), 2.76-2.54 (m, 5H), 2.30-1.84 (m, 12H), 1.65-1.50 (m, 4H), 1.03 (t, J=7.2 Hz, 3H).

### Synthesis D31: Synthesis of (3R)-3-[7-[2-cyano-3-[[ethyl(methyl)sulfamoyl]amino]-6-fluoro-phenoxy]quinoxalin-2-yl]-8-[4-[4-[(2,6-dioxo-3-piperidyl)amino]-2-fluoro-phenyl]cyclohexyl]-1-oxa-8-azaspiro[4.5]decane (Compound 60 and Compound 61)

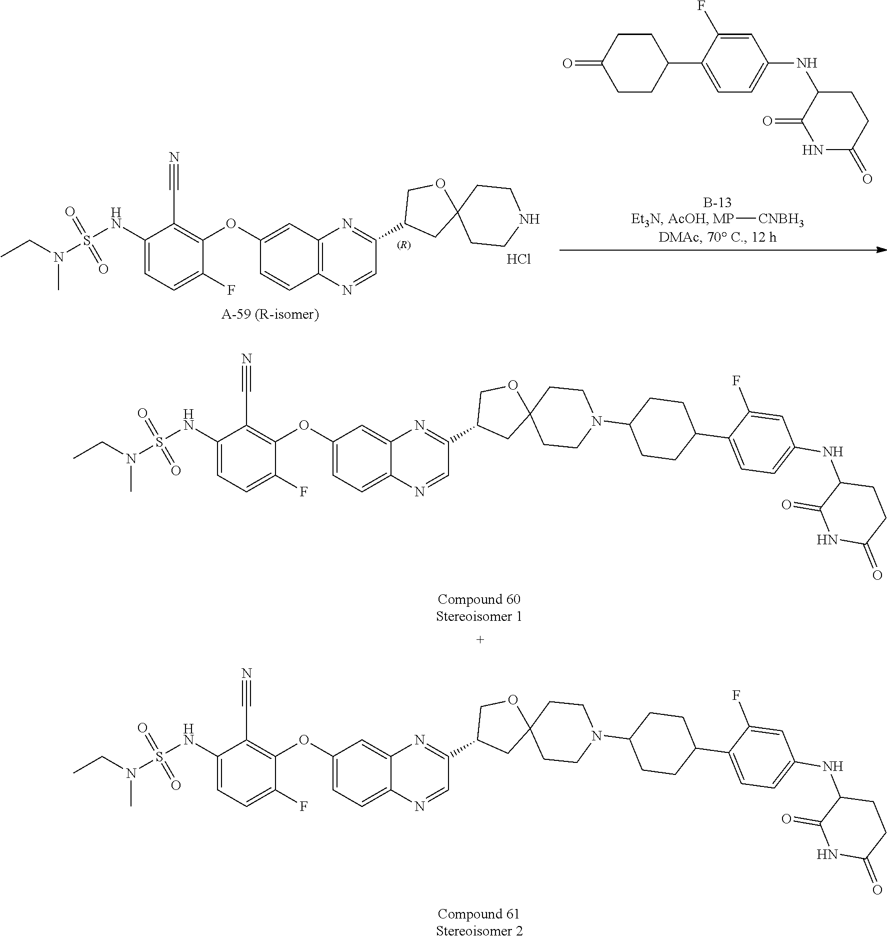

Into a 20 mL pressure vial containing a well-stirred solution of (3R)-3-[7-[2-cyano-3-[[ethyl(methyl)sulfamoyl]amino]-6-fluoro-phenoxy]quinoxalin-2-yl]-1-oxa-8-azaspiro[4.5]decane (A-59) (300 mg, 519.87 mol, HCl salt) in DMAc (5 mL) and was added triethylamine (1.56 mmol, 217.38 μL) at room temperature and mixture was stirred for 5 min at the same temperature. To the above mixture, AcOH (1.04 mmol, 59.52 μL) and 3-[3-fluoro-4-(4-oxocyclohexyl)anilino]piperidine-2,6-dione (B-13) (198.60 mg, 623.84 mol) were added and stirred for 2 hours at room temperature. Subsequently, MP-CNBH₃ (600 mg, 519.87 mol) was added at room temperature and resulting reaction mixture was stirred at 70° C. for 12 hours. After completion of the reaction, the reaction mixture was filtered through cotton and filtrate was concentrated under reduced pressure to get a crude residue, which was purified by prep HPLC [Column: SHIMPACK C18 (150 mm*19 mm*5 μm), Mobile phase A: 10 mM ammonium bicarbonate in milli-Q water; Mobile phase B: acetonitrile]. Both fractions from HPLC were lyophilized to get two isomers. Further these two isomers were analyzed by SFC and assigned as stereoisomer 1 (Peak-1 (t_{R}=2.01 min) by SFC sequence, Peak-2 by Prep HPLC sequence) and stereoisomer 2 (Peak-2 (t_{R}=3.18 min) by SFC sequence, Peak-1 by Prep HPLC sequence).

Peak-1 (by SFC sequence): (3R)-3-[7-[2-cyano-3-[[ethyl(methyl)sulfamoyl]amino]-6-fluoro-phenoxy]quinoxalin-2-yl]-8-[4-[4-[(2,6-dioxo-3-piperidyl)amino]-2-fluoro-phenyl]cyclohexyl]-1-oxa-8-azaspiro[4.5]decane (Compound 60, 65 mg, 74.56 mol, 14% yield) as a white solid. Peak-1 was assigned as stereoisomer 1 (chiral SFC t_{R}=2.01). UPLC-MS (ES+): m/z 843.7 [M+H]⁺. ¹H NMR (400 MHz, DMSO-d₆): δ 10.79 (s, 1H), 8.86 (s, 1H), 8.54 (bs, 1H), 8.14 (d, J=9.2 Hz, 1H), 7.70 (dd, J=2.8, 9.2 Hz, 1H), 7.48-7.40 (m, 1H), 7.38-7.28 (m, 1H), 7.15-7.05 (m, 2H), 6.52-6.40 (m, 2H), 6.03 (d, J=7.6 Hz, 1H), 4.33-4.29 (m, 2H), 4.20-3.99 (m, 2H), 3.60-3.50 (m, 1H), 3.20-2.80 (m, 5H), 2.78-2.65 (m, 1H), 2.67-2.55 (m, 4H), 2.40-2.20 (m, 2H), 2.10-1.58 (m, 15H), 1.0 (t, J=7.2 Hz, 3H).

Peak-2 (by SFC sequence): (3R)-3-[7-[2-cyano-3-[[ethyl(methyl)sulfamoyl]amino]-6-fluoro-phenoxy]quinoxalin-2-yl]-8-[4-[4-[(2,6-dioxo-3-piperidyl)amino]-2-fluoro-phenyl]cyclohexyl]-1-oxa-8-azaspiro[4.5]decane (Compound 61, 20 mg, 23.02 mol, 4% yield) as a white solid. Peak-2 was assigned as stereoisomer 2 (chiral SFC t_{R}=3.18 min). UPLC-MS (ES+): m/z 843.7 [M+H]⁺. ¹H NMR (400 MHz, DMSO-d₆): δ 10.78 (s, 1H), 9.23 (bs, 1H), 8.85 (s, 1H), 8.13 (d, J=8.8 Hz, 1H), 7.70 (dd, J=2.8, 9.2 Hz, 1H), 7.43-7.25 (m, 2H), 7.07 (d, J=1.6 Hz, 1H), 6.97 (t, J=9.2 Hz, 1H), 6.47-6.43 (m, 2H), 6.02 (d, J=7.6 Hz, 1H), 4.32-4.28 (m, 2H), 4.01-3.98 (m, 2H), 3.20-2.90 (m, 6H), 2.80-2.58 (m, 7H), 2.30-1.80 (m, 12H), 1.70-1.50 (m, 4H), 1.0 (t, J=6.8 Hz, 3H).

### Synthesis D32: Synthesis of (3R)-3-[6-[2-cyano-3-[[ethyl(methyl)sulfamoyl]amino]-6-fluoro-phenoxy]-4-oxo-quinazolin-3-yl]-8-[3-[4-[(2,6-dioxo-3-piperidyl)amino]-2-fluoro-phenyl]cyclobutyl-1-oxa-8-azaspiro[4.5]decane (Compound 62 and Compound 63)

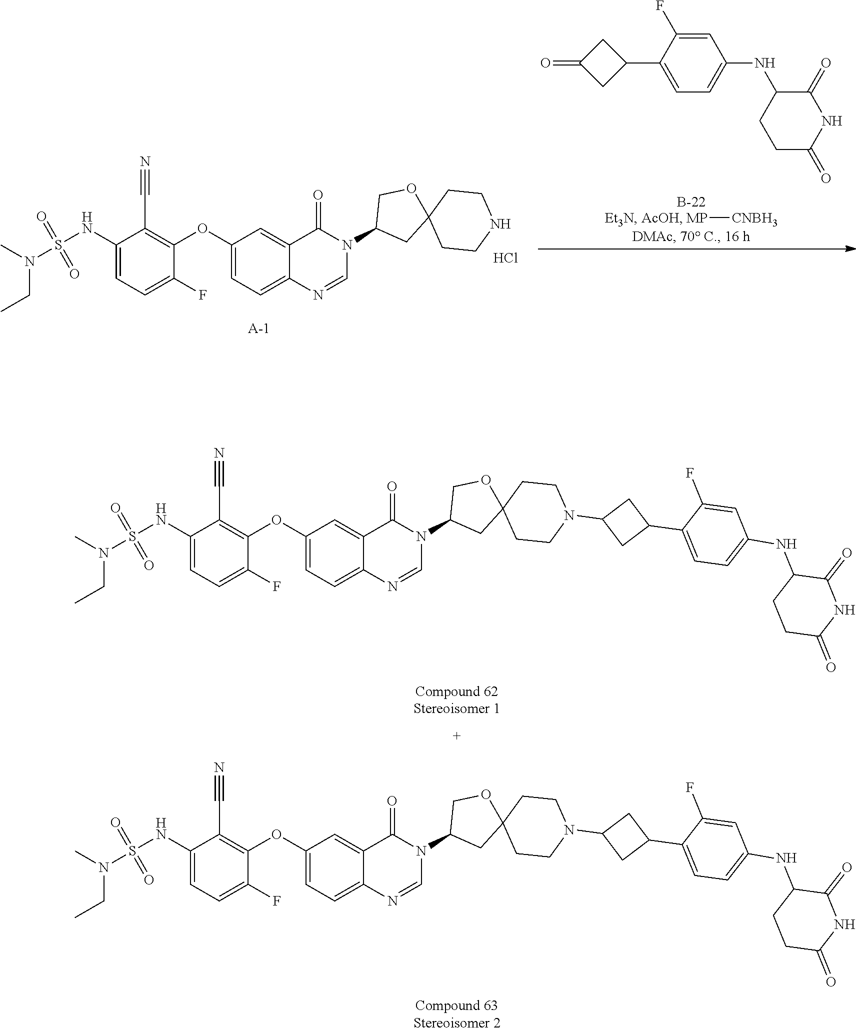

Into a 20 mL pressure vial containing a well-stirred solution of (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (A-1) (0.4 g, 667.71 mol, HCl salt) in DMAc (5.04 mL) was added triethylamine (3.34 mmol, 465.33 μL) at room temperature under nitrogen atmosphere and mixture was stirred for 20 min. Reaction mixture was cooled to 0° C. and was added 3-[3-fluoro-4-(3-oxocyclobutyl)anilino]piperidine-2,6-dione (B-22) (316.03 mg, 1.00 mmol) followed by AcOH (2.00 mmol, 114.67 μL) and resulting mixture was stirred for 3 hours at room temperature. Finally, MP-CNBH₃ (600 mg, 667.71 mol) was added, and the reaction mixture was stirred at 70° C. for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fractions were lyophilized to afford 280 mg of mixture of cis and trans isomers. Further cis and trans isomers were separated by preparative HPLC [Column: SHIMPACK C18 (250 mm*21.2 mm*5 m), Mobile phase A: 10 mM NH₄HCO₃ in milli-Q water; Mobile phase B: acetonitrile].

The first eluting fraction at (Peak-1) at t_{R}=2.49 min was concentrated under reduced pressure at 40° C. and lyophilized to afford Compound 62 (assigned as stereoisomer 1).

Peak-1: (3R)-3-[6-[2-cyano-3-[[ethyl(methyl)sulfamoyl]amino]-6-fluoro-phenoxy]-4-oxo-quinazolin-3-yl]-8-[3-[4-[(2,6-dioxo-3-piperidyl)amino]-2-fluoro-phenyl]cyclobutyl]-1-oxa-8-azaspiro[4.5]decane (Compound 62, 47.8 mg, 57.17 mol, 17% yield) as an off-white solid. UPLC-MS (ES+): m/z 831.2 [M+H]⁺. ¹H NMR (400 MHz, DMSO-d₆): δ 10.78 (s, 1H), 8.29 (s, 1H), 7.76 (d, J=8.80 Hz, 1H), 7.64 (dd, J=3.20, 8.80 Hz, 1H), 7.52-7.40 (m, 1H), 7.39-7.22 (m, 2H), 7.04 (t, J=8.80 Hz, 1H), 6.51 (dd, J=2.00, 8.40 Hz, 1H), 6.45-6.41 (m, 1H), 6.05 (d, J=8.00 Hz, 1H), 5.33-5.21 (m, 1H), 4.41-4.28 (m, 1H), 4.19-4.10 (m, 2H), 3.21-3.17 (m, 1H), 3.07-2.80 (m, 6H), 2.75-2.69 (m, 1H), 2.62-2.54 (m, 5H), 2.43-2.36 (m, 1H), 2.21-2.03 (m, 4H), 2.00-1.67 (m, 5H), 1.02 (t, J=7.20 Hz, 3H).

Similarly, the second eluted fraction (Peak-2) at t_{R}=2.67 min was concentrated under reduced pressure at 40° C. and lyophilized to afford Compound 63 (assigned as stereoisomer 2).

Peak-2: (3R)-3-[6-[2-cyano-3-[[ethyl(methyl)sulfamoyl]amino]-6-fluoro-phenoxy]-4-oxo-quinazolin-3-yl]-8-[3-[4-[(2,6-dioxo-3-piperidyl)amino]-2-fluoro-phenyl]cyclobutyl]-1-oxa-8-azaspiro[4.5]decane (Compound 63, (5.12 mg, 6.02 mol, 2% yield) as an off-white solid. UPLC-MS (ES+): m/z 831.2 [M+H]⁺. ¹H NMR (400 MHz, DMSO-d₆): δ 10.78 (s, 1H), 8.30 (s, 1H), 7.76 (d, J=8.80 Hz, 1H), 7.64 (dd, J=2.80, 9.20 Hz, 1H), 7.56-7.11 (m, 3H), 7.04 (t, J=8.80 Hz, 1H), 6.51 (d, J=8.80 Hz, 1H), 6.44 (d, J=13.20 Hz, 1H), 6.06 (d, J=7.60 Hz, 1H), 5.31-5.20 (m, 1H), 4.38-4.30 (m, 1H), 4.19-4.09 (m, 2H), 3.21-3.19 (m, 1H), 3.05-2.99 (m, 2H), 2.78-2.72 (m, 1H), 2.60 (s, 3H), 2.44-2.39 (m, 1H), 2.17-2.01 (m, 4H), 1.98-1.72 (m, 5H), 1.02 (t, J=7.20 Hz, 3H).

### Synthesis D33: Synthesis of (3R)-3-[6-[2-cyano-3-[[ethyl(methyl)sulfamoyl]amino]-6-fluoro-anilino]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 64 and Compound 65)

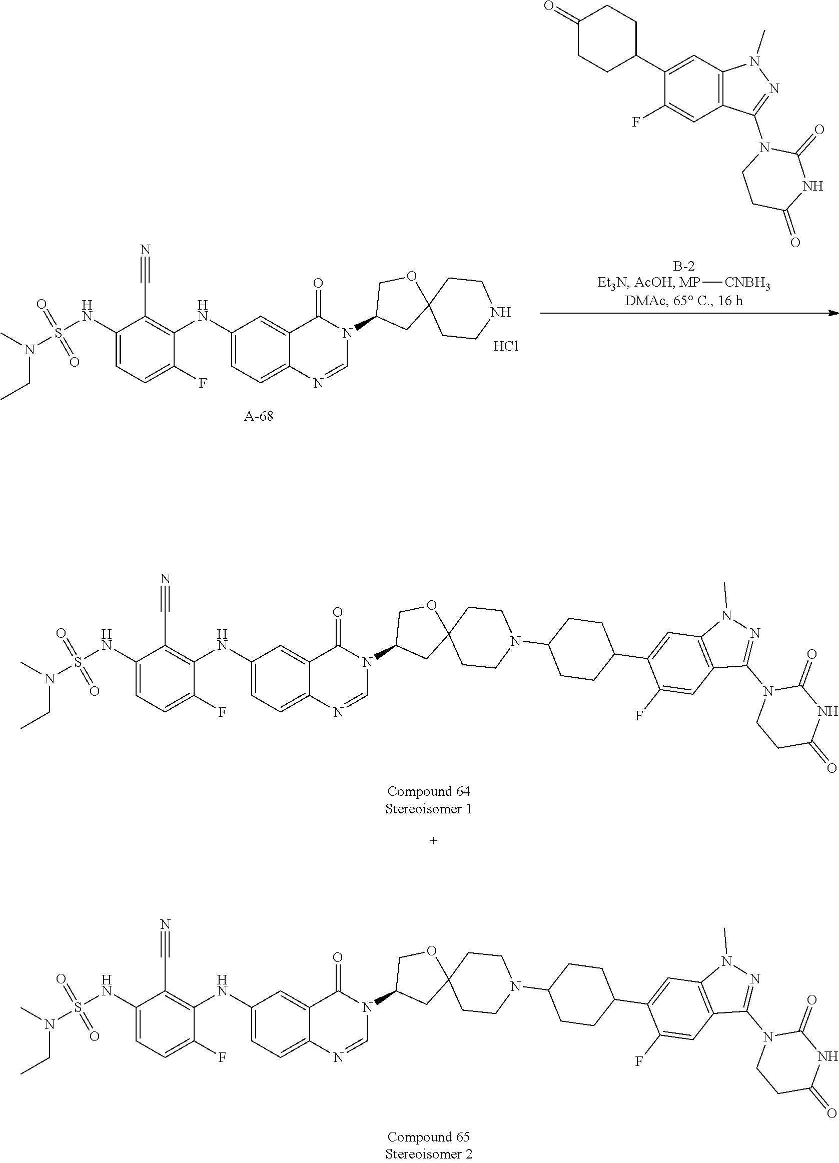

Into a 20 mL pressure vial containing a well stirred solution of (3R)-3-[6-[2-cyano-3-[[ethyl(methyl)sulfamoyl]amino]-6-fluoro-anilino]-4-oxo-quinazolin-3-yl]-1-oxa-8-azaspiro[4.5]decane (A-68) (200 mg, 337.79 mol, HCl salt) in DMAc (10 mL) was added triethylamine (337.79 mol, 47.08 μL) and stirred for 30 min at room temperature. After 30 min, 1-[5-fluoro-1-methyl-6-(4-oxocyclohexyl)indazol-3-yl]hexahydropyrimidine-2,4-dione (B-2) (121.05 mg, 337.79 mol) and AcOH (337.79 mol, 19.34 μL) were added and stirred for 1 h at room temperature. Subsequently, MP-CNBH₃ (150 mg, 337.79 mol) was added and resulting reaction mixture was stirred at 65° C. for 16 hours. After completion of the reaction, the reaction mixture was filtered through cotton pad and filtrate was concentrated under reduced pressure to get a crude residue which was purified by reverse phase column chromatography (Column: Redisep R_{f} Gold® reversed-phase, C18-Teledyne ISCO, 120 g, Mobile phase A: NH₄HCO₃ in milli-Q water; Mobile phase B: acetonitrile). Fractions were lyophilized to afford isomeric mixture of (3R)-3-[6-[2-cyano-3-[[ethyl(methyl)sulfamoyl]amino]-6-fluoro-anilino]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (300 mg, 213.81 mol, 63% yield) as an off-white solid.

The above isomeric mixture was purified by chiral SFC following the below method.

### Method of Analysis for Chiral SFC Separation

Instrument: PIC-22-016
Column: Ethyl pyridine (250 mm*30 mm*5 m)
Mobile Phase: CO₂: 0.5% IPAm in IPA [65:35]
Total Flow: 120 mL/min
Back pressure: 120 bar
Wavelength: 220 nm
Cycle time: 15 min

Fast eluting isomer (Peak-1): Fast eluting compound (Peak-1, t_{R}=2.79 min) was collected and concentrated under reduced pressure to afford (3R)-3-[6-[2-cyano-3-[[ethyl(methyl)sulfamoyl]amino]-6-fluoro-anilino]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 64, 44.98 mg, 48.38 mol, 14% yield) as an off-white solid.

Peak-1 was assigned as stereoisomer 1. UPLC-MS (ES+): m/z 898.2 [M+H]⁺. ¹H NMR (400 MHz, DMSO-d₆): δ 10.56 (s, 1H), 9.68 (bs, 1H), 8.76 (s, 1H), 8.20 (s, 1H), 7.58-7.20 (m, 7H), 5.38-5.28 (m, 1H), 4.15-4.08 (m, 2H), 4.02 (s, 3H), 3.91 (t, J=6.40 Hz, 2H), 3.12-2.98 (m, 3H), 2.80-2.68 (m, 6H), 2.45-2.32 (m, 2H), 2.12-1.60 (m, 14H), 1.04 (t, J=7.20 Hz, 3H).

Late eluting isomer (Peak-2): Late eluting compound (Peak-2, t_{R}=3.75 min) was collected and concentrated under reduced pressure to get an off-white solid (Peak-2), which contain traces of impurities in ¹H-NMR. Which was further purified by preparative HPLC [Column: X-Bridge C18 (250 mm*19 mm*5 m), Mobile phase A: 10 mm NH₄HCO₃ in milli-Q water; Mobile phase B: acetonitrile]. Fractions were lyophilized to afford Compound 65 (assigned as stereoisomer 2).

Peak-2: (3R)-3-[6-[2-cyano-3-[[ethyl(methyl)sulfamoyl]amino]-6-fluoro-anilino]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 65, 8.73 mg, 9.69 mol, 3% yield) as an off-white solid. UPLC-MS (ES+): m/z 898.5 [M+H]⁺. ¹H NMR (400 MHz, DMSO-d₆): δ 10.55 (s, 1H), 8.64 (s, 1H), 8.18 (s, 1H), 7.57-7.52 (m, 2H), 7.40-7.28 (m, 3H), 7.24-7.15 (m, 2H), 5.35-5.25 (m, 1H), 4.20-4.08 (m, 3H), 3.98 (s, 3H), 3.90 (t, J=6.80 Hz, 3H), 3.10-2.80 (m, 7H), 2.75 (t, J=6.80 Hz, 2H), 2.64 (s, 3H), 2.42-2.35 (m, 1H), 2.10-1.82 (m, 7H), 1.80-1.50 (m, 5H), 1.03 (t, J=7.20 Hz, 3H).

### Synthesis D34: Synthesis of (3R)-3-[6-[2-cyano-3-[[ethyl(methyl)sulfamoyl]amino]-6-fluoro-anilino]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 66 and Compound 67)

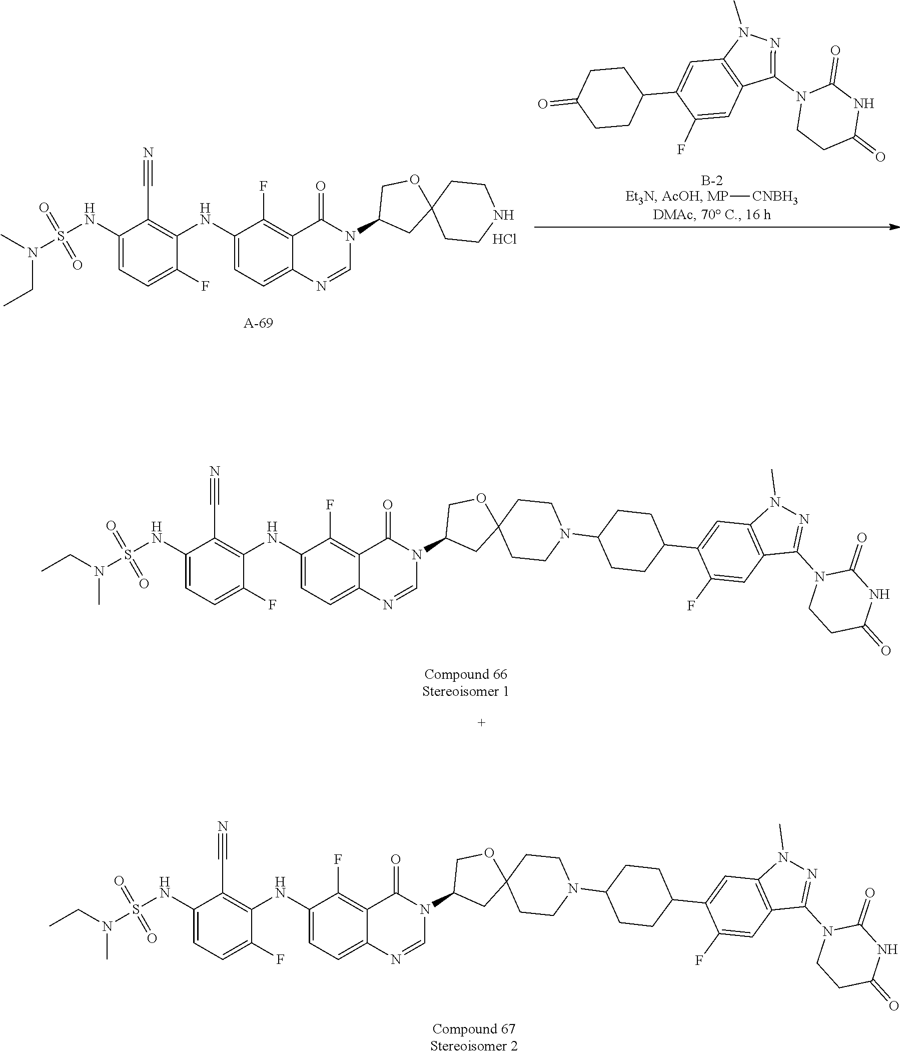

Into a 20 mL pressure capped vial containing a well stirred solution of (3R)-3-[6-[2-cyano-3-[[ethyl(methyl)sulfamoyl]amino]-6-fluoro-anilino]-5-fluoro-4-oxo-quinazolin-3-yl]-1-oxa-8-azaspiro[4.5]decane (A-69) (190.00 mg, 208.66 μmol, HCl salt) in anhydrous DMAc (2.5 mL) was added triethylamine (1.04 mmol, 145.42 μL) and stirred for 30 minutes. To the above mixture, 1-[5-fluoro-1-methyl-6-(4-oxocyclohexyl)indazol-3-yl]hexahydropyrimidine-2,4-dione (B-2) (89.73 mg, 250.40 μmol) and AcOH (625.99 mol, 35.83 μL) were added and stirred at room temperature for 2 hours. Subsequently, MP-CNBH₃ (300 mg, 208.66 μmol) was added and resulting mixture was stirred at 70° C. for 16 hours. After completion of the reaction, the reaction mixture was filtered through sintered funnel and the filtrate was concentrated under reduced pressure to get a crude residue which was purified by preparative HPLC [(Column: YMC ACTUS C18 (250 mm*20 mm*5 μm), mobile Phase A: 10 mM ammonium bicarbonate in water, mobile phase B: 100% acetonitrile]. The first eluting fraction (Peak-1) was concentrated under reduced pressure at 40° C. and lyophilized to afford Compound 66. Peak-1 was assigned as stereoisomer 1.

Peak-1:(3R)-3-[6-[2-cyano-3-[[ethyl(methyl)sulfamoyl]amino]-6-fluoro-anilino]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 66, 14 mg, 14.95 μmol, 7% yield) as an off-white solid. UPLC-MS (ES+): m/z 916.2 [M+H]⁺. ¹H NMR (400 MHz, DMSO-d₆): δ 10.55 (s, 1H), 8.38-8.36 (brs, 1H), 8.20 (s, 1H), 7.54 (d, J=6.00 Hz, 1H), 7.40-7.36 (m, 3H), 7.25-7.15 (m, 2H), 5.33-5.32 (m, 1H), 4.15-4.10 (m, 2H), 4.02-3.99 (m, 4H), 3.90 (t, J=6.80 Hz, 3H), 3.04 (q, J=6.80 Hz, 2H), 2.99-2.72 (m, 5H), 2.75 (t, J=6.80 Hz, 3H), 2.68-2.67 (m, 3H), 2.42-2.39 (m, 1H), 2.10-1.82 (m, 7H), 1.80-1.56 (m, 5H), 1.02 (t, J=76.00 Hz, 3H).

Similarly, the second eluted fraction (Peak-2) was concentrated under reduced pressure at 40° C. and lyophilized to afford Compound 67. Peak-2 was assigned as stereoisomer 2.

Peak-2: (3R)-3-[6-[2-cyano-3-[[ethyl(methyl)sulfamoyl]amino]-6-fluoro-anilino]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 67, 16 mg, 16.61 μmol, 8% yield) as an off-white solid. UPLC-MS (ES+): m/z 916.2 [M+H]⁺. ¹H NMR (400 MHz, DMSO-d₆): δ 10.55 (s, 1H), 8.50 (brs, 1H), 8.22 (s, 1H), 7.48 (d, J=6.00 Hz, 1H), 7.42-7.38 (m, 3H), 7.31-7.28 (m, 1H), 7.18-7.14 (m, 1H), 5.33-5.32 (m, 1H), 4.15-4.11 (m, 2H), 4.02 (s, 3H), 3.91 (t, J=6.80 Hz, 2H), 3.10-3.02 (m, 3H), 2.75 (t, J=6.80 Hz, 3H), 2.68-2.67 (m, 3H), 2.42-2.40 (m, 2H), 2.12-1.61 (m, 15H), 1.03 (t, J=6.80 Hz, 3H).

### Synthesis D35: Synthesis of (3S)-3-[6-[2-cyano-3-[[ethyl(methyl)sulfamoyl]amino]-6-fluoro-anilino]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 68 and Compound 69)

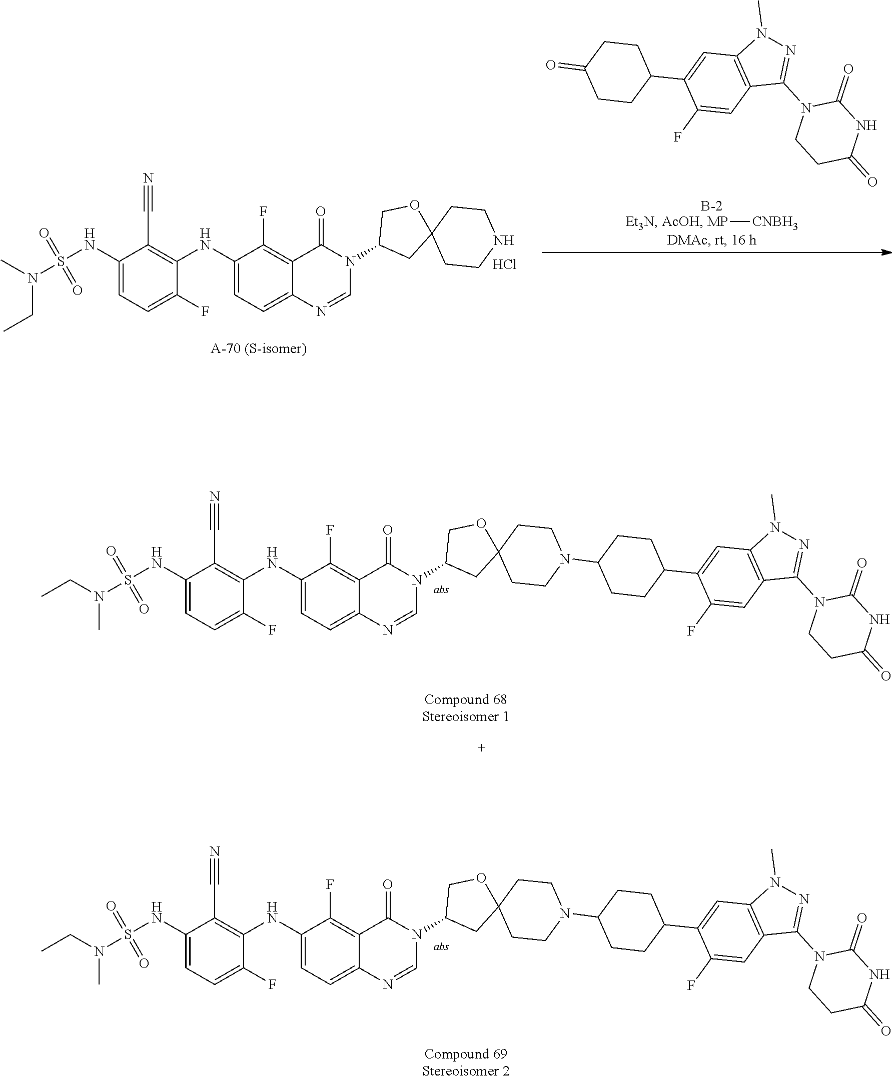

Into a 40 mL pressure vial containing a well-stirred solution of (3S)-3-[6-[2-cyano-3-[[ethyl(methyl)sulfamoyl]amino]-6-fluoro-anilino]-5-fluoro-4-oxo-quinazolin-3-yl]-1-oxa-8-azaspiro[4.5]decane (A-70) (200 mg, 281.93 mol, HCl salt) in anhydrous DMAc (3.02 mL) was added triethylamine (1.41 mmol, 196.48 μL) at room temperature under nitrogen atmosphere and mixture was stirred for 30 min. Reaction mixture was cooled to 0° C. and was added 1-[5-fluoro-1-methyl-6-(4-oxocyclohexyl)indazol-3-yl]hexahydropyrimidine-2,4-dione (B-2) (101.04 mg, 281.93 mol) followed by AcOH (845.80 mol, 48.42 μL) and resulting mixture was stirred for 3 hours at room temperature. Finally, MP-CNBH₃ (400 mg, 281.93 mol) was added to the above mixture and stirred at room temperature for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fractions were lyophilized to afford 50 mg of mixture of cis and trans isomers. Further cis and trans isomers were separated by preparative HPLC [Column: X-Bridge C18 (250 mm*19 mm*5 m), Mobile phase A: 10 mM NH₄HCO₃ in milli-Q water; Mobile phase B: acetonitrile]. First eluting fraction (Peak-1) was concentrated under reduced pressure at 40° C. and lyophilized to afford Compound 68. Peak-1 was assigned as stereoisomer 1.

Peak-1: (3S)-3-[6-[2-cyano-3-[[ethyl(methyl)sulfamoyl]amino]-6-fluoro-anilino]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 68, 14 mg, 14.64 Pmol, 5% yield). UPLC-MS (ES+): m/z 916.2 [M+H]⁺. ¹H NMR (400 MHz, DMSO-d₆): δ 10.55 (s, 1H), 8.40 (s, 1H), 8.20 (d, J=7.20 Hz, 1H), 7.54 (d, J=6.00 Hz, 1H), 7.46-7.32 (m, 3H), 7.31-7.21 (m, 1H), 7.20-7.07 (m, 1H), 5.31 (quint, J=5.60 Hz, 1H), 4.20-4.11 (m, 2H), 4.00 (s, 3H), 3.90 (t, J=6.80 Hz, 2H), 3.09-3.02 (m, 2H), 3.01-2.81 (m, 5H), 2.75 (t, J=6.80 Hz, 3H), 2.72-2.61 (m, 4H), 2.47-2.36 (m, 2H), 2.21-1.86 (m, 8H), 1.71-1.58 (m, 3H), 1.02 (t, J=6.80 Hz, 3H).

Similarly, the second eluted fraction (Peak-2) was concentrated under reduced pressure at 40° C. and lyophilized to afford Compound 69. Peak-2 was assigned as stereoisomer 2.

Peak-2: (3S)-3-[6-[2-cyano-3-[[ethyl(methyl)sulfamoyl]amino]-6-fluoro-anilino]-5-fluoro-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 69, 15 mg, 16.01 Pmol, 6% yield). UPLC-MS (ES+): m/z 916.3 [M+H]⁺. ¹H NMR (400 MHz, DMSO-d₆): δ 10.55 (s, 1H), 8.53 (s, 1H), 8.22 (s, 1H), 7.48 (d, J=6.00 Hz, 1H), 7.46-7.34 (m, 3H), 7.7.33-7.24 (m, 1H), 7.17 (dd, J=4.40, 9.20 Hz, 1H), 5.30 (quint, J=5.60 Hz, 1H), 4.21-4.09 (m, 2H), 4.02 (s, 3H), 3.91 (t, J=7.2 Hz, 2H), 3.12-2.99 (m, 4H), 2.75 (t, J=6.80 Hz, 3H), 2.69 (s, 3H), 2.48-2.37 (m, 1H), 2.22-1.41 (m, 14H), 1.03 (t, J=7.20 Hz, 3H).

### Synthesis D36: Synthesis of (3S)-3-[6-[2-Cyano-3-[[ethyl(methyl)sulfamoyl]amino]-6-fluoro-phenoxy]-4-oxo-quinazolin-3-yl]-8-[3-[3-(2,4-dioxohexahydropyrimidin-1-yl)-5-fluoro-1-methyl-indazol-6-yl]cyclobutyl]-1-oxa-8-azaspiro[4.5]decane (Compound 70)

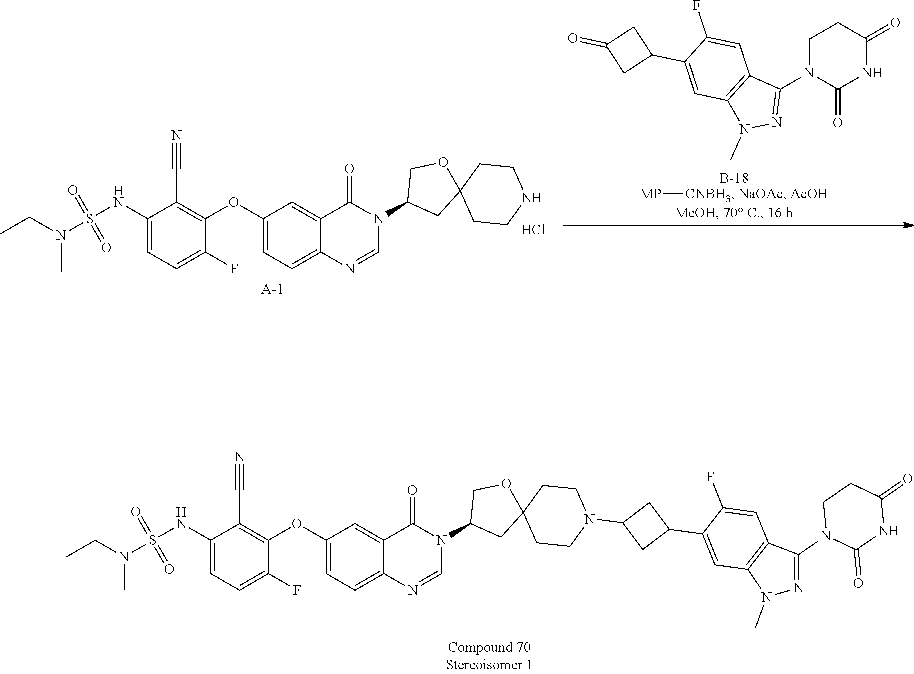

Into a 15 mL pressure vial containing a well stirred solution of (3S)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane 1 (A-1) (56.37 mg, 95.05 μmol, HCl salt) in methanol (1 mL) was added NaOAc (38.98 mg, 475.24 μmol) and stirred for 30 minutes. To the above mixture, 1-[5-fluoro-1-methyl-6-(3-oxocyclobutyl)indazol-3-yl]hexahydropyrimidine-2,4-dione 2 (B-18) (43.10 mg, 95.05 μmol, TFA salt) and AcOH (285.15 mol, 16.32 μL) were added and stirred at room temperature for 2 hours. Subsequently, MP-CNBH₃ (100 mg, 95.05 μmol) was added at room temperature and the resulting mixture was stirred at 70° C. for 16 hours. After completion of the reaction, the reaction mixture was filtered through celite pad and filtrate was concentrated under reduced pressure to get a crude residue, which was purified by prep HPLC [Column: X-Bridge C8 (250 mm*19 mm*5 μm); Mobile Phase A: 10 mM ammonium bicarbonate in milli-Q water; Mobile Phase B: 100% acetonitrile] to afford (45 mg) of mixture of cis and trans-isomers which was further purified by chiral SFC following the below method.

### Method of Analysis for Chiral SFC Separation

Instrument: PIC 22-016
Column: Ethyl pyridine (250*30) mm, 5 m
Mobile Phase: CO₂: 0.5% IPAm in IPA[65:35]
Total Flow: 100 mL/min
Back pressure: 120 bar
Wavelength: 220 nm
Cycle time: 7 min

The obtained mixture compound was again purified by preparative HPLC (Column: X-Bridge C18 (150 mm*19 mm*5 μm), mobile Phase A: 10 mM ammonium bicarbonate in milli-Q water; mobile Phase B: 100% acetonitrile). The pure fractions were lyophilized to afford (3S)-3-[6-[2-cyano-3-[[ethyl(methyl)sulfamoyl]amino]-6-fluoro-phenoxy]-4-oxo-quinazolin-3-yl]-8-[3-[3-(2,4-dioxohexahydropyrimidin-1-yl)-5-fluoro-1-methyl-indazol-6-yl]cyclobutyl]-1-oxa-8-azaspiro[4.5]decane (Compound 70, 16 mg, 18.32 μmol, 36.75% yield) as an off-white solid. The compound was assigned as stereoisomer 1 as per SFC (Rₜ: 2.02 min, chiral SFC Purity: 100%). UPLC-MS (ES+): m/z 871.2 [M+H]⁺. ¹H NMR (400 MHz, DMSO-d₆): δ 10.56 (s, 1H), 9.70 (bs, 1H), 8.32 (s, 1H), 7.77 (d, J=8.80 Hz, 1H), 7.65 (dd, J=3.20, 8.80 Hz, 1H), 7.52-7.50 (m, 2H), 7.39-7.33 (m, 3H), 5.30-5.27 (m, 1H), 4.18-4.09 (m, 2H), 4.02 (s, 3H), 3.91 (t, J=6.80 Hz, 2H), 3.45-3.41 (m, 2H), 3.34-3.33 (m, 1H), 3.04 (q, J=6.80 Hz, 2H), 2.88-2.65 (m, 5H), 2.69-2.61 (m, 5H), 2.42-2.35 (m, 1H), 2.26-2.05 (m, 3H), 1.94-1.70 (m, 4H), 1.03 (t, J=7.20 Hz, 3H).

### Synthesis D37: Synthesis of (3R)-8-[4-[3-(2,4-dioxohexahydropyrimidin-1-yl)-5-fluoro-1-methyl-indazol-6-yl]cyclohexyl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 71 and Compound 72)

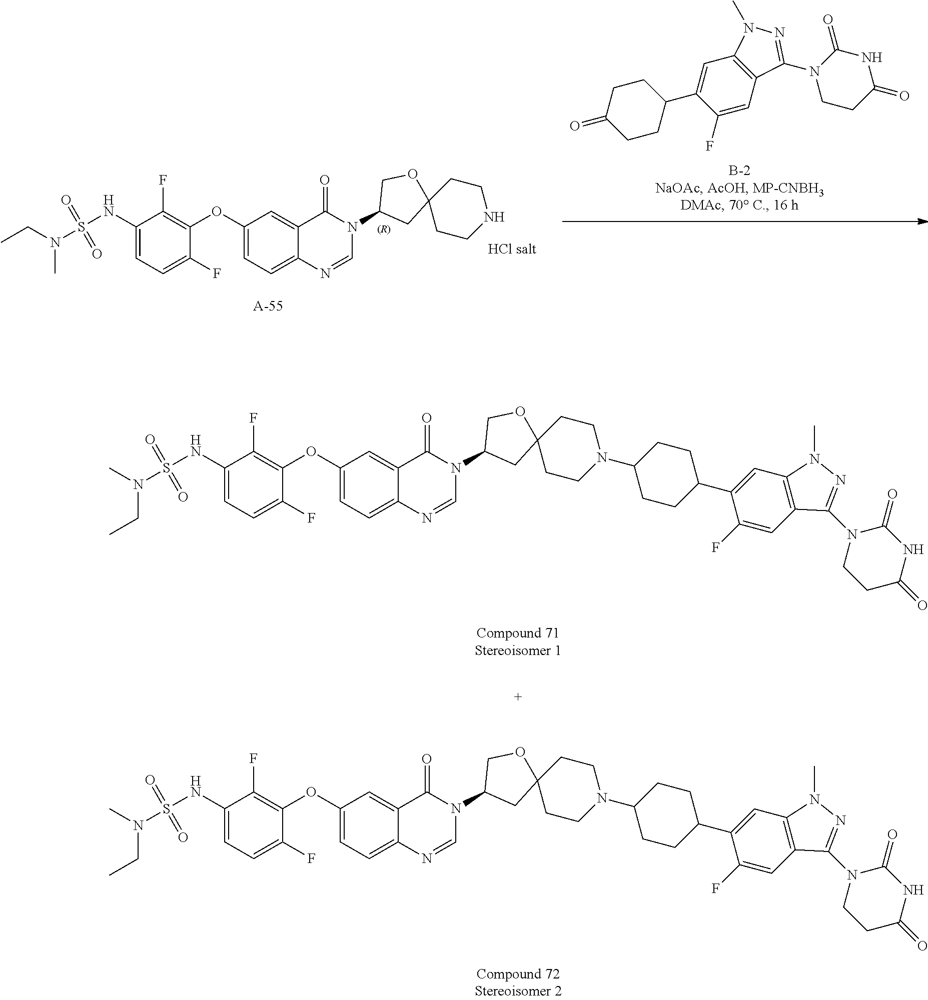

Into a 40 mL pressure vial containing a well stirred solution of (3R)-3-[6-[3-[[ethyl(methyl)sulfamoyl]amino]-2,6-difluoro-phenoxy]-4-oxo-quinazolin-3-yl]-1-oxa-8-azaspiro[4.5]decane (A-55) (500 mg, 853.17 mol, HCl salt) in DMAc (10.01 mL) was added anhydrous NaOAc (209.96 mg, 2.56 mmol) at room temperature under nitrogen atmosphere and mixture was stirred for 30 minutes. Reaction mixture was cooled to 0° C. and was added 1-[5-fluoro-1-methyl-6-(4-oxocyclohexyl)indazol-3-yl]hexahydropyrimidine-2,4-dione (B-2) (305.75 mg, 853.17 mol) followed by acetic acid (4.27 mmol, 243.96 μL) and resulting mixture was stirred for 3 hours at room temperature. Finally, MP-CNBH₃ (500 mg, 853.17 mol) was added, and the reaction mixture was stirred at 70° C. for 16 hours. After completion of the reaction, the reaction mixture was filtered, and filtrate was concentrated under reduced pressure to get a crude residue (500 mg, 218.62 mol, 26% yield), which was purified by achiral SFC.

### Method of Analysis for Achiral SFC Separation

Column: Diol
Mobile Phase: CO₂: 0.5% IPAm in IPA (75:25)
Run time: 10.00 min
Flow: 4.0 mL/min,
Co-Solvent: 25.0% IPAm,
Oven Temperature: 40° C.
Wavelength: 220 nm
BPR Pressure: 100.0 BPV

The first eluting fraction (Peak-1) at t_{R}=3.10 min was concentrated under reduced pressure at 40° C. and lyophilized to afford Compound 71 (assigned as stereoisomer 1).

Peak-1: (3R)-8-[4-[3-(2,4-dioxohexahydropyrimidin-1-yl)-5-fluoro-1-methyl-indazol-6-yl]cyclohexyl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 71, 78 mg, 84.29 mol, 15% yield) as an off-white solid. UPLC-MS (ES+): m/z 892.2 [M+H]. ¹HNMR (400 MHz, DMSO-d₆): δ 10.55 (s, 1H), 9.60 (bs, 1H), 8.34 (s, 1H), 7.78 (d, J=8.8 Hz, 1H), 7.67 (dd, J=3.20, 9.00 Hz, 1H), 7.54 (d, J=6.00 Hz, 1H), 7.42-7.27 (m, 4H), 5.31-5.17 (m, 1H), 4.18-4.02 (m, 2H), 3.98 (s, 3H), 3.93 (t, J=6.4 Hz, 2H), 3.09 (q, J=7.2 Hz, 2H), 2.90-2.77 (m, 1H), 2.77-2.68 (m, 7H), 2.61-2.53 (m, 2H), 2.32-2.28 (m, 1H), 2.08-1.87 (m, 5H), 1.78-1.55 (m, 6H), 1.56-1.40 (m, 2H), 1.01 (t, J=7.20 Hz, 3H).

Similarly, the second eluted fraction (Peak-2) at t_{R}=4.53 min was concentrated under reduced pressure at 40° C. and lyophilized to afford Compound 72 (assigned as stereoisomer 2).

Peak-2: (3R)-8-[4-[3-(2,4-dioxohexahydropyrimidin-1-yl)-5-fluoro-1-methyl-indazol-6-yl]cyclohexyl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 72, 55 mg, 58.52 mol, 10% yield) as an off-white solid. UPLC-MS (ES+): m/z 892.3 [M+H]⁺. ¹H NMR (400 MHz, DMSO-d₆): δ 10.54 (s, 1H), 9.74 (bs, 1H), 8.33 (s, 1H), 7.78 (d, J=9.20 Hz, 1H), 7.67 (dd, J=2.80, 9.00 Hz, 1H), 7.54 (d, J=6.00 Hz, 1H), 7.49-7.30 (m, 4H), 5.32-5.17 (m, 1H), 4.13-4.05 (m, 2H), 3.98 (s, 3H), 3.90 (t, J=6.80 Hz, 2H), 3.09 (q, J=7.2 Hz, 2H), 2.89-2.71 (m, 8H), 2.61-2.55 (m, 1H), 2.39-2.28 (m, 1H), 2.18-1.86 (m, 5H), 1.84-1.36 (m, 8H), 1.02 (t, J=7.20 Hz, 3H).

### Synthesis D38: Synthesis of (3R)-3-[6-[2-cyano-6-fluoro-3-[[2-hydroxyethyl(methyl)sulfamoyl]amino]phenoxy]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 73 and Compound 74)

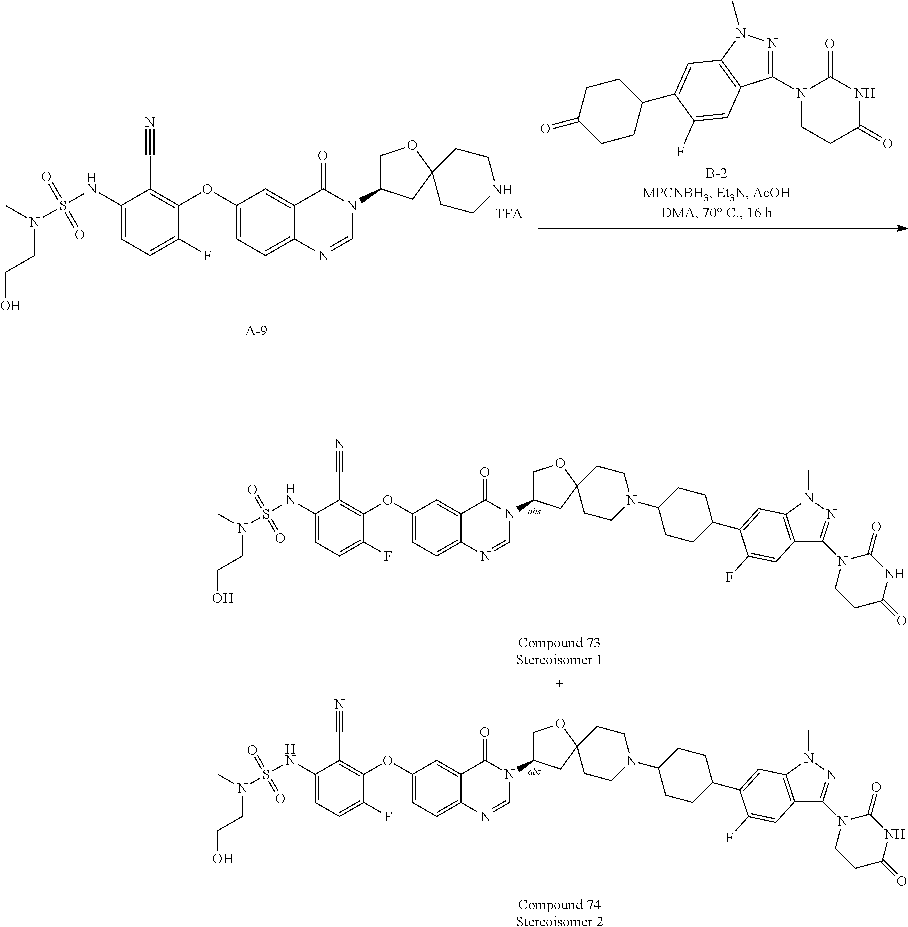

Into a 20 mL pressure vial containing the well-stirred solution of (3R)-3-[6-[2-cyano-6-fluoro-3-[[2-hydroxyethyl(methyl)sulfamoyl]amino]phenoxy]-4-oxo-quinazolin-3-yl]-1-oxa-8-azaspiro[4.5]decane (A-9) (300 mg, 318.95 mol, TFA salt) in DMAc (4.05 mL) was added triethylamine (637.90 mol, 88.91 μL) at room temperature and stirred for 30 minutes at the same temperature. To the above mixture, AcOH (956.85 mol, 54.78 μL) and 1-(5-fluoro-1-methyl-6-(4-oxocyclohexyl)-1H-indazol-3-yl)dihydropyrimidine-2,4(1H,3H)-dione (B-2) (136.07 mg, 350.84 μmol) were added at 0° C. and stirred at room temperature for 2 hours. Subsequently, MP-CNBH₃ (300 mg, 318.95 μmol) was added and stirred at 70° C. for 16 hours. After completion of the reaction, the reaction mixture was filtered through a cotton pad and the filtrate was concentrated under reduced pressure to get a crude residue which was purified by prep HPLC [Column: X-Bridge C18 (250 mm*19 mm*5 m), Mobile phase A: 10 mm NH₄HCO₃ in milli-Q water; Mobile phase B: acetonitrile]. Both fractions from HPLC were collected separately and lyophilized to get two isomers. Further these two isomers were analyzed by SFC as stereoisomer 1 (Peak-1 (t_{R}=3.14 min) by SFC sequence, Peak-2 by Prep HPLC sequence) and stereoisomer 2 (Peak-2 (t_{R}=4.08 min) by SFC sequence, Peak-1 by Prep HPLC sequence).

Peak-1 (by SFC sequence): (3R)-3-[6-[2-cyano-6-fluoro-3-[[2-hydroxyethyl(methyl)sulfamoyl]amino]phenoxy]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 73, 24.59 mg, 26.50 mol, 17% yield). UPLC-MS: m/z 915.4 [M+H]⁺. ¹H NMR (400 MHz, DMSO-d₆): δ 10.55 (s, 1H), 8.65 (bs, 1H), 8.32 (s, 1H), 7.76 (d, J=8.8 Hz, 1H), 7.59 (dd, J=2.80, 8.80 Hz, 1H), 7.49-7.34 (m, 5H), 5.35-5.25 (m, 1H), 4.90-4.30 (m, 1H), 4.21-4.08 (m, 2H), 4.25 (s, 3H), 3.90 (t, J=6.8 Hz, 2H), 3.49 (t, J=6.8 Hz, 2H), 3.20-2.85 (m, 5H), 2.76 (t, J=6.80 Hz, 2H), 2.68 (s, 3H), 2.41-2.35 (m, 1H), 2.20-1.70 (m, 13H).

Peak-2 (by SFC sequence): (3R)-3-[6-[2-cyano-6-fluoro-3-[[2-hydroxyethyl((methyl)sulfamoyl]amino]phenoxy]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 74, 25.20 mg, 26.83 mol, 17% yield) as off-white solid. UPLC-MS: m/z 915.4 [M+H]⁺. ¹H NMR (400 MHz, DMSO-d₆): δ 10.55 (s, 1H), 9.51 (bs, 1H), 8.31 (d, J=6.40 Hz, 1H), 7.76 (d, J=8.80 Hz, 1H), 7.64 (dd, J=3.20, 9.00 Hz, 1H), 7.39-7.27 (m, 5H), 5.35-5.25 (m, 1H), 4.90-4.10 (m, 3H), 4.00 (s, 3H), 3.93 (t, J=6.40 Hz, 2H), 3.49 (t, J=6.40 Hz, 2H), 3.21-2.81 (m, 7H), 2.74 (t, J=6.40 Hz, 2H), 2.67 (s, 3H), 2.48-2.38 (m, 1H), 2.20-1.60 (m, 13H).

### Synthesis D39: Synthesis of (3R)-3-[6-[2-cyano-6-fluoro-3-[[2-hydroxyethyl(methyl)sulfamoyl]amino]phenoxy]-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 75 and Compound 76)

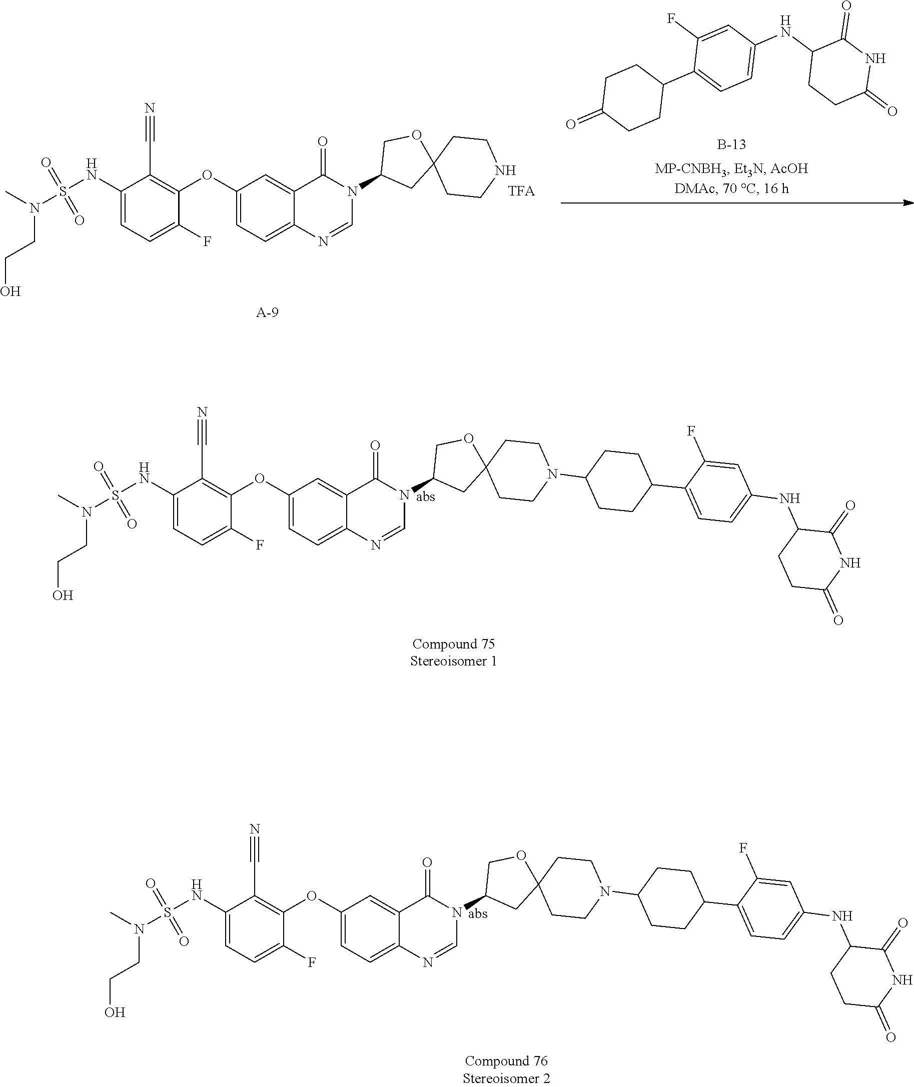

A well-stirred solution of (3R)-3-[6-[2-cyano-6-fluoro-3-[[2-hydroxyethyl(methyl)sulfamoyl]amino]phenoxy]-4-oxo-quinazolin-3-yl]-1-oxa-8-azaspiro[4.5]decane (A-9) (300 mg, 318.95 mol, TFA salt) in DMAc (4.05 mL) in a 10 mL glass vial with pressure released cap was cooled to 0° C. and triethylamine (637.90 mol, 88.91 μL) was added. The mixture was stirred for 30 minutes at room temperature. To the above mixture, AcOH (956.85 mol, 54.78 μL) followed by 1-(5-fluoro-1-methyl-6-(4-oxocyclohexyl)-1H-indazol-3-yl)dihydropyrimidine-2,4(1H,3H)-dione (B-13) (136.07 mg, 350.84 μmol) were added and resulting mixture was stirred for 2 hours at room temperature. Subsequently, MP-CNBH₃ (300 mg, 318.95 μmol) was added, and reaction mixture was stirred at 70° C. for 16 hours. After completion of the reaction, the reaction mixture was filtered through cotton pad and filtrate was concentrated under reduced pressure to obtain (3R)-3-[6-[2-cyano-6-fluoro-3-[[2-hydroxyethyl(methyl)sulfamoyl]amino]phenoxy]-4-oxo-quinazolin-3-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (300 mg, 157.38 mol, 49.34% yield, 48% purity) as a brown oil which was purified by preparative HPLC to separate mixture of cis and trans isomers [Column: X-Bridge C18 (250*19 mm*5 m), Mobile phase A: 10 mM ammonium bicarbonate in milli-Q water; Mobile phase B: acetonitrile]. The first eluting fraction (Peak-1) was concentrated under reduced pressure at 40° C. and lyophilized to afford Compound 75 (assigned as stereoisomer 1).

Peak-1: (3R)-3-[6-[2-cyano-6-fluoro-3-[[2-hydroxyethyl(methyl)sulfamoyl]amino]phenoxy]-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 75, 30.60 mg, 34.31 mol, 18% yield). UPLC-MS (ES+): m/z 875.2 [M+H]⁺. ¹H NMR (400 MHz, DMSO-d₆): δ 10.78 (s, 1H), 9.08 (bs, 1H), 8.31 (s, 1H), 7.75 (d, J=8.8 Hz, 1H), 7.63 (dd, J=2.8, 8.8 Hz, 1H), 7.37-7.33 (m, 2H), 7.28-7.26 (m, 1H), 6.96 (t, J=8.8 Hz, 1H), 6.46-6.42 (m, 2H), 6.02 (d, J=8.00 Hz, 1H), 5.32-5.24 (m, 1H), 4.35-4.24 (m, 1H), 4.22-4.08 (m, 2H), 3.49 (t, J=6.4 Hz, 2H), 3.32-3.01 (m, 6H), 2.73-2.67 (m, 6H), 2.49-2.30 (m, 1H), 2.09-1.82 (m, 11H), 1.55-1.49 (m, 4H).

Similarly, the second eluted fraction (Peak-2) was concentrated under reduced pressure at 40° C. and lyophilized to afford Compound 76 (assigned as stereoisomer 2).

Peak-2: (3R)-3-[6-[2-cyano-6-fluoro-3-[[2-hydroxyethyl(methyl)sulfamoyl]amino]phenoxy]-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 76, 14.42 mg, 16.35 mol, 9% yield). UPLC-MS (ES+): m/z 875.5 [M+H]⁺. ¹H NMR (400 MHz, DMSO-d₆): δ 10.79 (s, 1H), 8.61 (bs, 1H), 8.32 (s, 1H), 7.76 (d, J=9.6 Hz, 1H), 7.64 (dd, J=2.8, 8.8 Hz, 1H), 7.43-7.30 (m, 3H), 7.08 (bs, 1H), 6.52-6.43 (m, 2H), 6.02 (d, J=7.60 Hz, 1H), 5.30-5.27 (m, 1H), 4.32-4.29 (m, 1H), 4.17-4.11 (m, 2H), 3.49 (t, J=6.4 Hz, 2H), 3.07-3.05 (m, 3H), 2.95-2.62 (m, 2H), 2.74-2.67 (m, 6H), 2.49-2.32 (m, 1H), 2.10-1.63 (m, 16H).

### Synthesis D40: Synthesis of (3R)-8-[4-[4-[(2,6-dioxo-3-piperidyl)amino]-2-fluoro-phenyl]cyclohexyl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 77 and Compound 78)

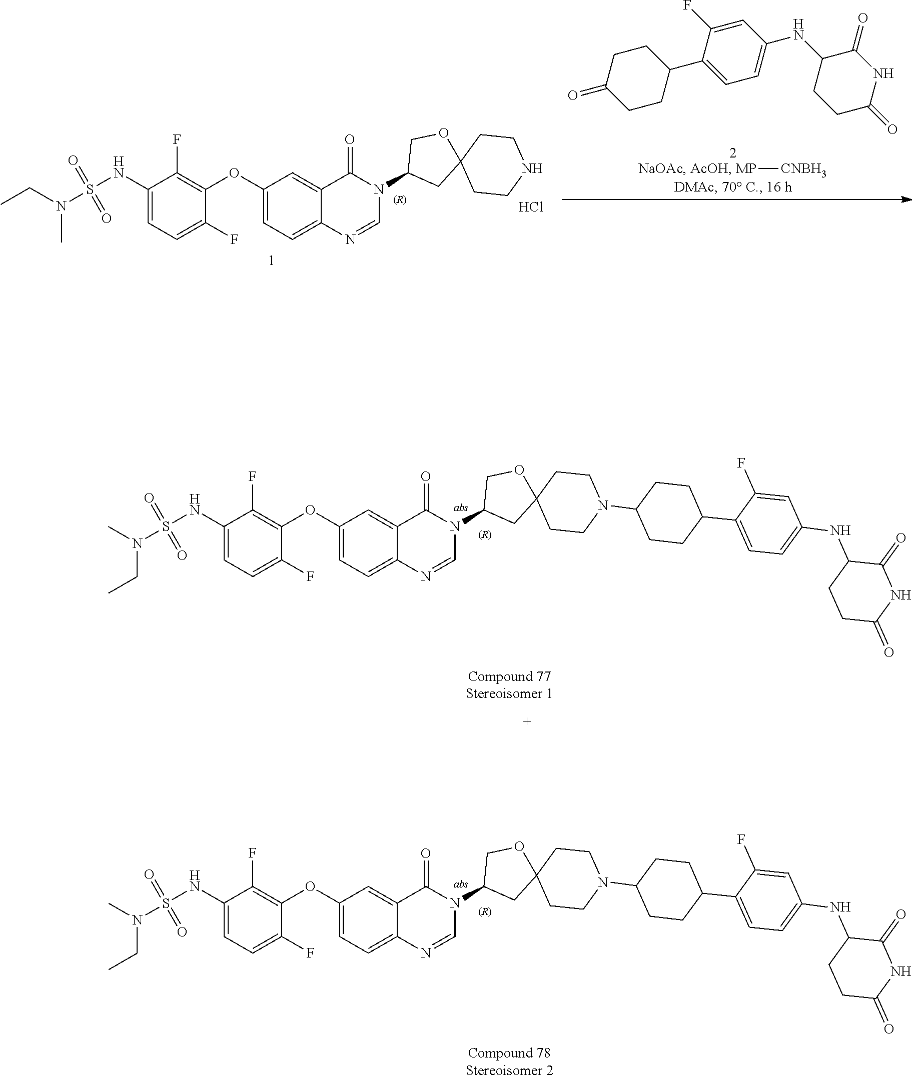

Into a 10 mL pressure vial containing a well stirred solution of (3R)-3-[6-[3-[[ethyl(methyl)sulfamoyl]amino]-2,6-difluoro-phenoxy]-4-oxo-quinazolin-3-yl]-1-oxa-8-azaspiro[4.5]decane 1 (A-55) (150 mg, 255.95 mol, HCl salt) in DMAc (2.01 mL) was added anhydrous NaOAc (62.99 mg, 767.85 mol) at room temperature under nitrogen atmosphere and mixture was stirred for 20 min. Reaction mixture was cooled to 0° C. and was added 3-[3-fluoro-4-(4-oxocyclohexyl)anilino]piperidine-2,6-dione 2 (B-13) (81.48 mg, 255.95 mol) followed by AcOH (1.28 mmol, 73.19 μL) and resulting mixture was stirred for 3 hours at room temperature. Finally, MP-CNBH₃ (150 mg, 255.95 mol) was added, and reaction mixture was stirred at 70° C. for 16 hours. After the completion of the reaction, the reaction mixture was filtered and filtrate was concentrated under reduced pressure to get a crude residue, which was purified by reverse phase column chromatography (Redisep R_{f} Gold®reversed-phase C18-teledyne ISCO, 120 g), [Mobile phase A: NH₄HCO₃ in milli-Q water; Mobile phase B: acetonitrile]. The fractions were lyophilized to afford a mixture of cis and trans isomers. Further cis and trans isomers were separated by preparative HPLC. [Column: X-Bridge C8 (250 mm*19 mm*5 m), Mobile phase A: 10 mM NH₄HCO₃ in milli-Q water; Mobile phase B: acetonitrile]. The first eluting fraction (Peak-1) was concentrated under reduced pressure at 40° C. and lyophilized to afford Compound 77 (assigned as stereoisomer 1).

Peak-1: (3R)-8-[4-[4-[(2,6-dioxo-3-piperidyl)amino]-2-fluoro-phenyl]cyclohexyl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 77, 13 mg, 14.61 mol, 6% yield) as an off-white solid. UPLC-MS (ES+): m/z 852.0 [M+H]⁺. ¹H NMR (400 MHz, DMSO-d₆): δ 10.78 (s, 1H), 9.74 (bs, 1H), 8.33 (s, 1H), 7.77 (d, J=8.80 Hz, 1H), 7.67 (dd, J=3.2, 9.2 Hz, 1H), 7.50-7.26 (m, 3H), 6.97 (t, J=8.80 Hz, 1H), 6.47-6.37 (m, 2H), 5.97 (d, J=7.6 Hz, 1H), 5.32-5.17 (m, 1H), 4.35-4.19 (m, 1H), 4.15-4.06 (m, 2H), 3.09 (q, J=7.6 Hz, 2H), 2.78-2.61 (m, 4H), 2.14-2.02 (m, 2H), 2.02-1.57 (m, 15H), 1.57-1.30 (m, 5H), 1.04 (t, J=6.8 Hz, 3H).

Similarly, the second eluted fraction (Peak-2) was concentrated under reduced pressure at 40° C. and lyophilized to afford Compound 78 (assigned as stereoisomer 2).

Peak-2: (3R)-8-[4-[4-[(2,6-dioxo-3-piperidyl)amino]-2-fluoro-phenyl]cyclohexyl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 78, 22 mg, 25.62 mol, 10% yield) as an off-white solid. UPLC-MS (ES+): m/z 852.2 [M+H]⁺. ¹H NMR (400 MHz, DMSO-d₆): δ 10.78 (s, 1H), 8.34 (s, 1H), 7.77 (d, J=8.80 Hz, 1H), 7.66 (dd, J=3.20, 9.2 Hz, 1H), 7.45-7.23 (m, 3H), 6.98 (t, J=8.80 Hz, 1H), 6.52-6.38 (m, 2H), 5.97 (d, J=8.00 Hz, 1H), 5.37-5.20 (m, 1H), 4.36-4.21 (m, 1H), 4.13-3.99 (m, 2H), 3.07 (q, J=7.2 Hz, 2H), 2.82-2.68 (m, 7H), 2.62-2.58 (m, 2H), 2.43-2.38 (m, 1H), 2.34-2.31 (m, 1H), 2.25-2.14 (m, 1H), 2.15-2.07 (m, 1H), 2.04-1.92 (m, 3H), 1.89-1.60 (m, 7H), 1.52-1.36 (m, 4H), 1.01 (t, J=7.20 Hz, 3H).

### Synthesis D41: Synthesis of 6-[2-Cyano-6-fluoro-3-[[2-hydroxyethyl(methyl)sulfamoyl]amino]phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 79 and Compound 80)

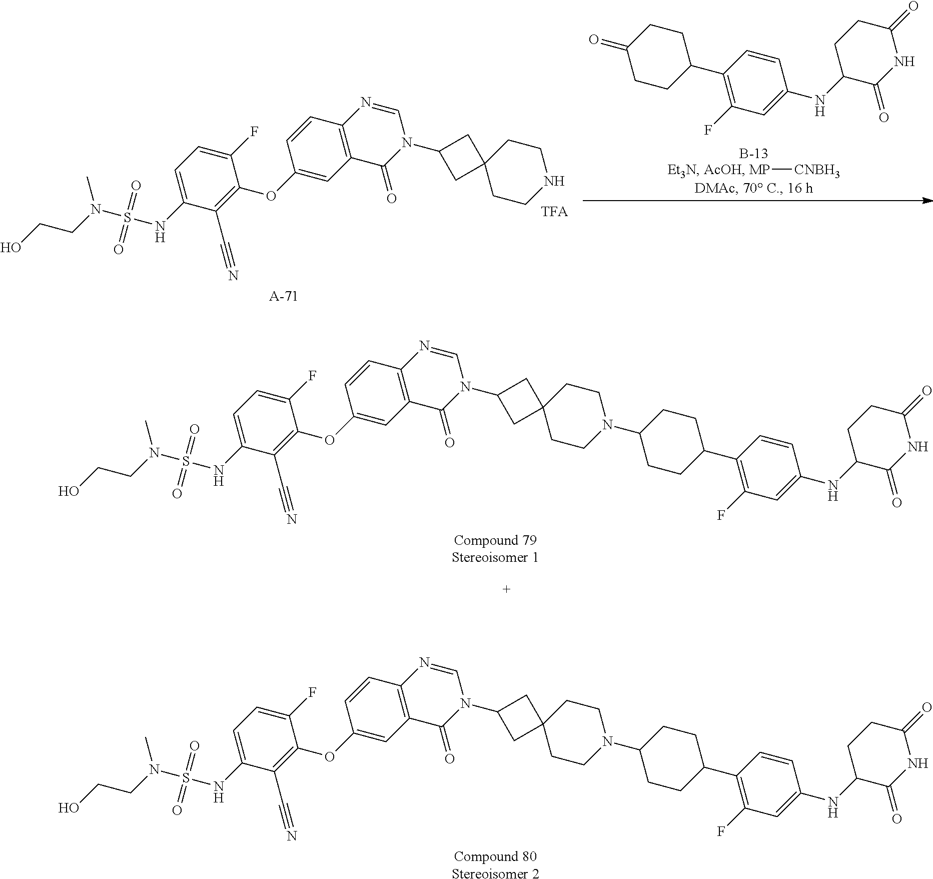

Into a 20 mL pressure vial containing the well-stirred solution of (7-azaspiro[3.5]nonan-2-yl)-6-[2-cyano-6-fluoro-3-[[2-hydroxyethyl(methyl)sulfamoyl]amino]phenoxy]-4-oxo-quinazoline (A-71) (404.37 mg, 298.23 mol, TFA salt) in DMAc (5 mL) was added NaOAc (73.39 mg, 894.68 mol) at room temperature and stirred for 30 minutes at the same temperature. To the above mixture, 3-[3-fluoro-4-(4-oxocyclohexyl)anilino]piperidine-2,6-dione (B-13) (94.94 mg, 298.23 mol) and AcOH (298.23 mol, 17.05 μL) were added at 0° C. and resulting reaction mixture was stirred at room temperature for 3 hours. Subsequently, MP-CNBH₃ (200 mg, 298.23 mol) was added and stirred at 70° C. for 16 hours. After the completion of the reaction, the reaction mixture was filtered through a cotton pad and the filtrate was concentrated under reduced pressure to get a crude residue which was purified by preparative HPLC [Column: X-SELECT CSH (150 mm*19 mm*5 μm), Mobile phase A: 10 mM NH₄HCO₃ in acetonitrile; Mobile phase B: 100% acetonitrile]. Fast eluting fraction (Peak-1) was lyophilized to afford Compound 79 (assigned as stereoisomer 1).

Peak-1: 6-[2-cyano-6-fluoro-3-[[2-hydroxyethyl(methyl)sulfamoyl]amino]phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 79, 6.43 mg, 7.35 mol, 2% yield) as an off-white solid. UPLC-MS: m/z 859.5 [M+H]⁺. ¹H NMR (400 MHz, DMSO-d₆): δ 10.79 (s, 1H), 8.91 (bs, 1H), 8.38 (s, 1H), 7.75 (d, J=8.80 Hz, 1H), 7.61 (dd, J=3.20, 9.00 Hz, 1H), 7.34 (d, J=3.20 Hz, 2H), 7.26 (s, 1H), 6.97 (t, J=8.40 Hz, 1H), 6.46-6.43 (s, 2H), 6.02 (d, J=7.60 Hz, 1H), 5.00-4.92 (m, 1H), 4.75-4.40 (m, 1H), 4.35-4.25 (m, 1H), 3.49 (t, J=6.40 Hz, 2H), 3.28-3.22 (m, 2H), 3.21-2.85 (m, 5H), 2.80-2.72 (m, 1H), 2.65-2.58 (m, 6H), 2.42-2.35 (m, 2H), 2.18-2.00 (m, 4H), 1.98-1.70 (m, 7H), 1.65-1.45 (m, 4H).

Similarly, the second eluted fraction (Peak-2) was lyophilized to afford Compound 80 (assigned as stereoisomer 2).

Peak-2: 6-[2-cyano-6-fluoro-3-[[2-hydroxyethyl(methyl)sulfamoyl]amino]phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 80, 6.69 mg, 7.72 mol, 3% yield) as an off-white solid. UPLC-MS: m/z 859.5 [M+H]⁺. ¹H NMR (400 MHz, DMSO-d₆): δ 10.80 (s, 1H), 8.48 (bs, 1H), 8.38 (s, 1H), 7.76 (d, J=9.20 Hz, 1H), 7.62 (dd, J=3.20, 9.00 Hz, 1H), 7.50-7.22 (m, 3H), 7.09 (s, 1H), 6.49-6.44 (m, 2H), 6.03 (d, J=7.60 Hz, 1H), 5.05-4.90 (m, 1H), 4.70-4.40 (m, 1H), 4.35-4.25 (m, 1H), 3.50 (t, J=6.40 Hz, 2H), 3.28-3.21 (m, 1H), 3.19-2.82 (m, 5H), 2.80-2.65 (m, 4H), 2.62-2.52 (m, 3H), 2.40-2.31 (m, 3H), 2.20-1.46 (m, 15H).

### Synthesis D42: Synthesis of 6-[2-cyano-6-fluoro-3-[[2-hydroxyethyl(methyl)sulfamoyl]amino]phenoxy]-3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazoline (Compound 81 and Compound 82)

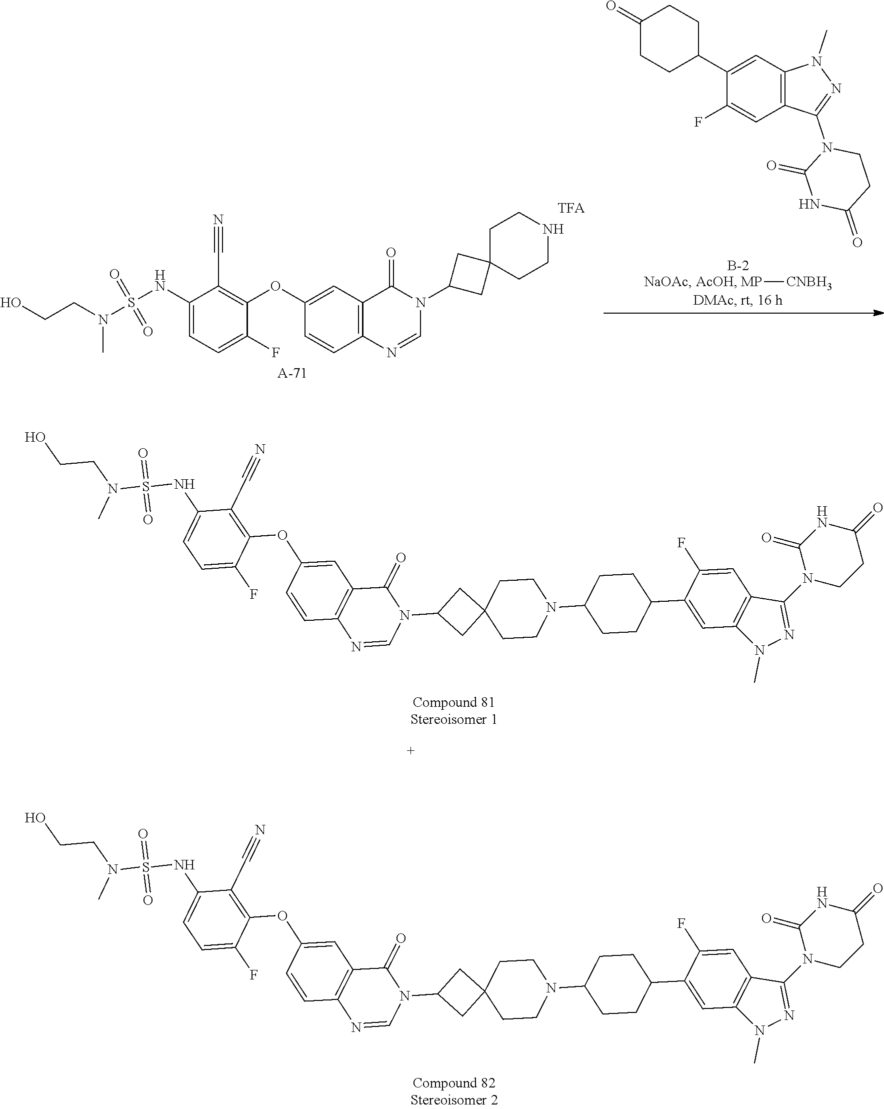

Into a 20 mL pressure vial containing the well-stirred solution of 3-(7-azaspiro[3.5]nonan-2-yl)-6-[2-cyano-6-fluoro-3-[[2-hydroxyethyl(methyl)sulfamoyl]amino]phenoxy]-4-oxo-quinazoline (A-71) (450 mg, 671.01 mol, TFA salt) in DMAc (5.02 mL) was added NaOAc (165.13 mg, 2.01 mmol) at room temperature and stirred for 30 min at the same temperature. To the above mixture 1-[5-fluoro-1-methyl-6-(4-oxocyclohexyl)indazol-3-yl]hexahydropyrimidine-2,4-dione (B-2) (168.33 mg, 469.71 mol) and AcOH (3.36 mmol, 191.87 μL) were added at 0° C. and resulting reaction mixture was stirred at room temperature for 3 hours. Subsequently, MP-CNBH₃ (450 mg, 671.01 mol) was added and stirred at room temperature for 16 hours. After completion of the reaction, the reaction mixture was filtered through a cotton pad and the filtrate was concentrated under reduced pressure to get a crude residue which was purified by preparative HPLC [Column: YMC Actus c18 (250 mm*20 mm*5 μm), Mobile phase A: 10 mM NH₄HCO₃ in acetonitrile; Mobile phase B: 100% acetonitrile]. The fast-eluting fraction was lyophilized to afford Compound 81 (assigned as stereoisomer 1).

Peak-1: 6-[2-cyano-6-fluoro-3-[[2-hydroxyethyl(methyl)sulfamoyl]amino]phenoxy]-3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazoline (Compound 81, 12 mg, 12.97 mol, 5% yield) as an off-white solid. UPLC-MS: m/z 899.2 [M+H]⁺. ¹H NMR (400 MHz, DMSO-d₆): δ 10.55 (s, 1H), 9.03 (bs, 1H), 8.38 (s, 1H), 7.76 (d, J=8.80 Hz, 1H), 7.62 (dd, J=2.80, 9.00 Hz, 1H), 7.54 (dd, J=6.00 Hz, 1H), 7.39-7.26 (m, 4H), 4.97-4.93 (m, 1H), 4.03 (s, 3H), 3.90 (t, J=6.80 Hz, 2H), 3.49 (t, J=6.40 Hz, 2H), 3.28-3.25 (m, 2H), 3.23-2.80 (m, 7H), 2.75 (t, J=6.80 Hz, 2H), 2.65 (s, 3H), 2.42-2.32 (m, 4H), 2.22-2.10 (m, 2H), 2.07-1.75 (m, 6H), 1.73-1.60 (m, 4H). Similarly, the second eluted fraction was lyophilized to afford Compound 82 (assigned as stereoisomer 2).

Peak-2: 6-[2-cyano-6-fluoro-3-[[2-hydroxyethyl(methyl)sulfamoyl]amino]phenoxy]-3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazoline (Compound 82, 16 mg, 17.09 mol, 6% yield) as an off-white solid. UPLC-MS: m/z 899.6 [M+H]⁺. ¹H NMR (400 MHz, DMSO-d₆): δ 10.56 (s, 1H), 8.60 (s, 1H), 8.39 (s, 1H), 7.76 (d, J=8.80 Hz, 1H), 7.64-7.30 (m, 6H), 4.97-4.93 (m, 1H), 4.80-4.30 (m, 1H), 4.03 (s, 3H), 3.91 (t, J=6.80 Hz, 2H), 3.60-3.48 (m, 3H), 3.22-3.82 (m, 5H), 2.76 (t, J=6.80 Hz, 2H), 2.68-2.60 (m, 4H), 2.42-2.30 (m, 4H), 2.26-1.60 (m, 13H).

### Synthesis D43: Synthesis of 6-[2-cyano-3-[[ethyl(methyl)sulfamoyl]amino]-6-fluoro-phenoxy]-3-[7-[4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 83 and Compound 84)

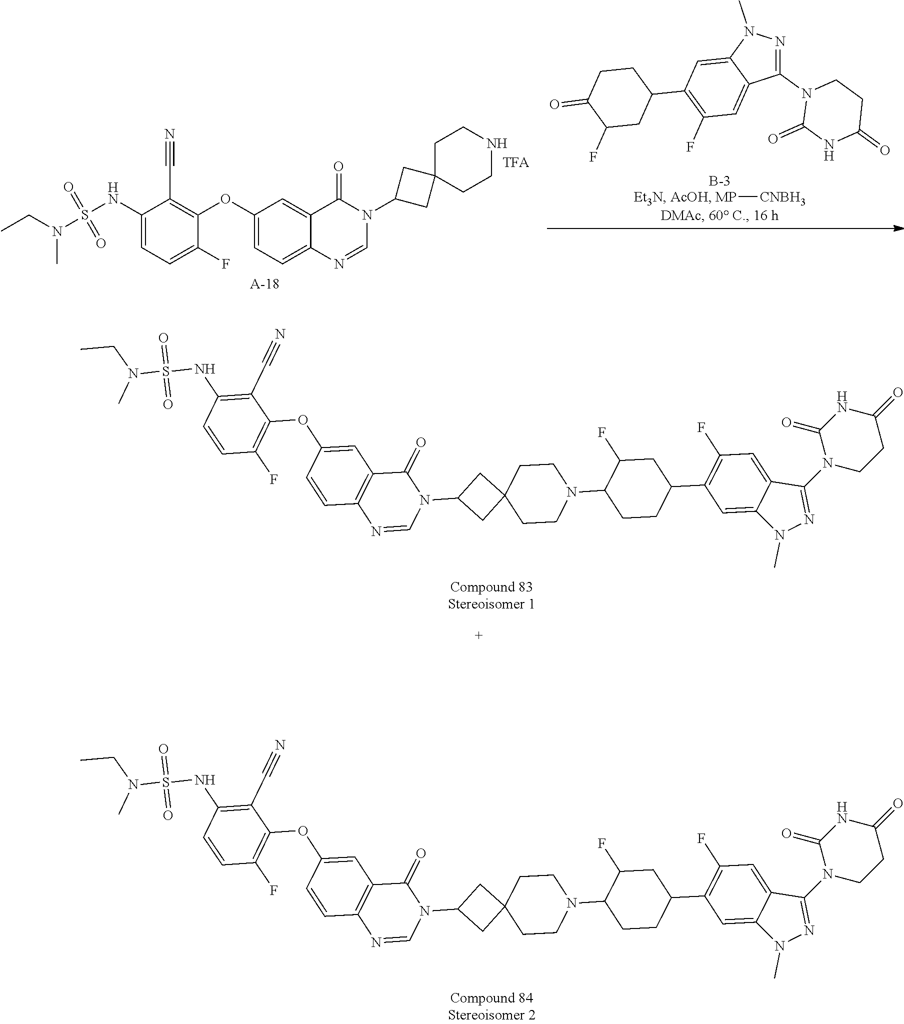

Into a 20 mL glass vial containing a well stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline 1 (A-18) (0.500 g, 763.79 mol, TFA salt) and 1-[5-fluoro-6-(3-fluoro-4-oxo-cyclohexyl)-1-methyl-indazol-3-yl]hexahydropyrimidine-2,4-dione 2 (B-3) (287.46 mg, 763.79 mol) in DMAc (5.0 mL) was added triethylamine (2.29 mmol, 319.37 μL) at room temperature and resulting mixture was stirred at the same temperature for 1 hour. After 1 h, AcOH (763.79 mol, 43.68 μL) was added and stirring was continued at room temperature for 2 hours. Subsequently, MP-CNBH₃ (1.0 g, 763.79 mol) was added and resulting reaction mixture was stirred at 60° C. for 16 hours. After the completion of the reaction, the reaction mixture was filtered through cotton pad and pad was washed with dichloromethane (10 mL). Filtrate was concentrated under reduced pressure to get a crude residue which was purified by preparative HPLC (Column: X BRIDGE C18 (150 mm*21 mm*5 m), Mobile phase A: 10 mM NH₄HCO₃ (pH was adjust to 10 with Ammonia) in H₂O, Mobile phase B: 100% acetonitrile, flow: 15 mL/min). The first eluting fraction (Peak-1) was lyophilized to afford Compound 83 (assigned as stereoisomer 1).

Peak-1: 6-[2-cyano-3-[[ethyl(methyl)sulfamoyl]amino]-6-fluoro-phenoxy]-3-[7-[4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 83, 0.030 g, 33.22 mol, 4% yield) as a white solid. UPLC-MS (ES+): m/z 901.2 [M+H]⁺. ¹H NMR (400 MHz, DMSO-d₆): δ 10.56 (s, 1H), 9.75 (bs, 1H), 8.40 (s, 1H), 7.78 (d, J=9.20 Hz, 1H), 7.66-7.58 (m, 3H), 7.41-7.34 (m, 3H), 5.50-5.28 (m, 1H), 5.00-4.90 (m, 1H), 4.00 (s, 3H), 3.91 (t, J=6.80 Hz, 2H), 3.25-3.20 (m, 1H), 3.18-2.81 (m, 6H), 2.76 (t, J=6.80 Hz, 2H), 2.68 (s, 3H), 2.40-2.30 (m, 4H), 2.28-2.18 (m, 1H), 2.10-1.70 (m, 10H), 1.04 (t, J=7.20 Hz, 3H).

Similarly, the second eluted was lyophilized to afford Compound 84 (assigned as stereoisomer 2).

Peak-2: 6-[2-cyano-3-[[ethyl(methyl)sulfamoyl]amino]-6-fluoro-phenoxy]-3-[7-[4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 84, 0.020 g, 22.16 mol, 3% yield) as a white solid. UPLC-MS (ES+): m/z 901.2 [M+H]⁺. ¹H NMR (400 MHz, DMSO-d₆): δ 10.56 (s, 1H), 10.12 (s, 1H), 8.41 (s, 1H), 7.79-7.65 (m, 3H), 7.52 (d, J=6.00 Hz, 1H), 7.48-7.41 (m, 1H), 7.40-7.35 (m, 2H), 5.30-5.10 (m, 1H), 4.95-4.89 (m, 1H), 4.03 (s, 3H), 3.91 (t, J=6.80 Hz, 2H), 3.13 (q, J=6.80 Hz, 2H), 2.77-2.74 (m, 5H), 2.65-2.55 (m, 2H), 2.40-2.31 (m, 2H), 2.30-2.11 (m, 3H), 2.10-1.60 (m, 10H), 1.04 (t, J=4.00 Hz, 3H).

### Synthesis D44: Synthesis of 6-[2-cyano-3-[[ethyl(methyl)sulfamoyl]amino]-6-fluoro-phenoxy]-3-[7-[(2S)-4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 85) and 6-[2-cyano-3-[[ethyl(methyl)sulfamoyl]amino]-6-fluoro-phenoxy]-3-[7-[(2R)-4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 86)

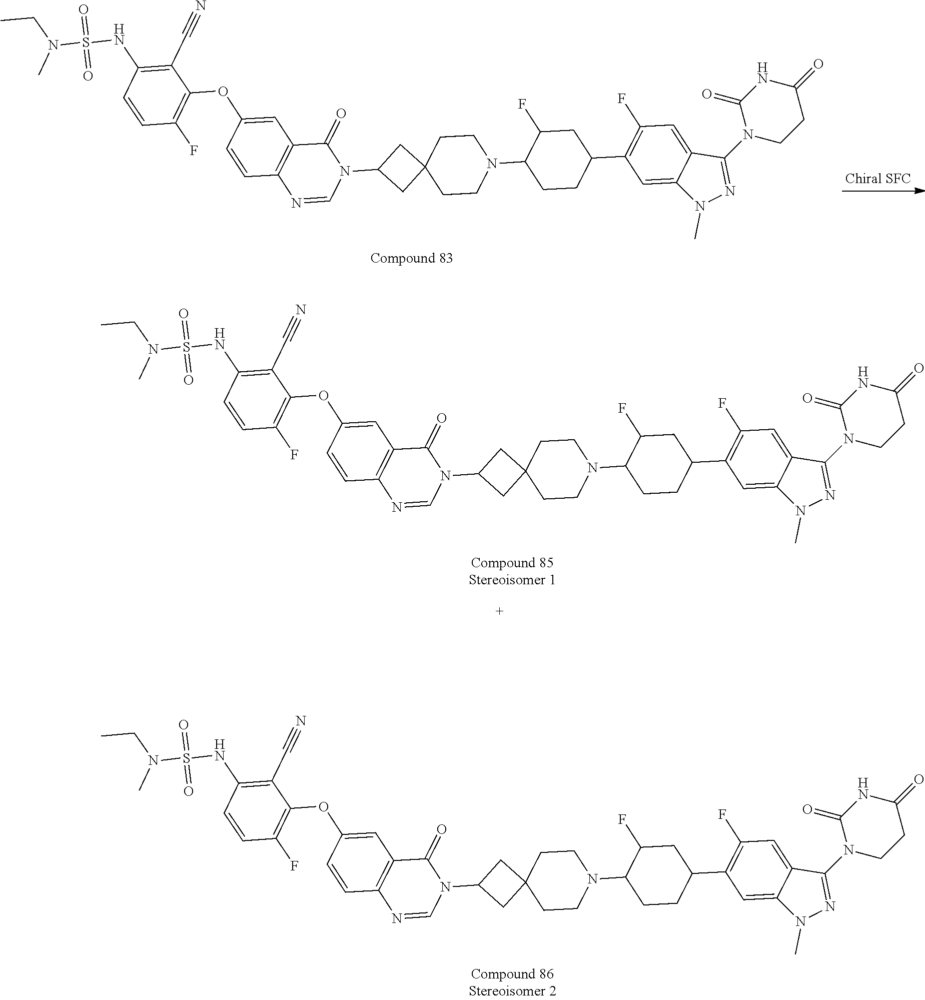 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N ; CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N ; CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N]

Compound 83 (40 mg) following the same procedure as above was further separated by chiral SFC following the below method.

### Method of Analysis for Chiral SFC Separation

Instrument: PIC-22-017
Column: I Cellulose Z(250*30)mm, 5 m
Mobile Phase: C02: 0.5% IPAm in methanol: ACN [50:50]
Total Flow: 100 ml/min
Back pressure: 100 bar
Wavelength: 220 nm
Cycle time: 15 min

Fast eluting isomer (Peak-1): Fast eluting compound (Peak-1, t_{R}=9.43 min) was collected and concentrated under reduced pressure to afford 6-[2-cyano-3-[[ethyl(methyl)sulfamoyl]amino]-6-fluoro-phenoxy]-3-[7-[(4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (5 mg, 5.46 mol, 1% yield) as an off-white solid. Peak-1 was assigned as stereoisomer 1. UPLC-MS (ES+): m/z 901.3 [M+H]⁺. ¹H NMR (400 MHz, DMSO-d₆): δ 10.56 (s, 1H), 9.77 (bs, 1H), 8.40 (s, 1H), 7.77 (d, J=9.20 Hz, 1H), 7.66-7.59 (m, 3H), 6 7.37 (dd, J=2.80, 20.20 Hz, 3H), 5.65-5.16 (m, 1H), 4.99-4.90 (m, 1H), 4.00 (s, 3H), 3.91 (t, J=6.80 Hz, 2H), 3.33-3.21 (m, 1H), 3.07-2.74 (m, 8H), 2.72-2.68 (m, 3H), 2.42-2.37 (m, 2H), 2.29-2.19 (m, 2H), 2.07-1.72 (m, 9H), 1.04 (t, J=7.20 Hz, 3H).

Late eluting isomer (Peak-2): Late eluting compound (Peak-2, t_{R}=10.66 min) was collected and concentrated under reduced pressure to afford 6-[2-cyano-3-[[ethyl(methyl)sulfamoyl]amino]-6-fluoro-phenoxy]-3-[7-[(4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (3 mg, 3.17 mol, 0.4% yield) as an off-white solid. Peak-2 was assigned as stereoisomer 2. UPLC-MS (ES+): m/z 901.6 [M+H]⁺. ¹H NMR (400 MHz, DMSO-d₆): δ 10.56 (s, 1H), 8.40 (s, 1H), 7.77 (d, J=9.20 Hz, 1H), 7.70-7.60 (m, 2H), 7.59-7.31 (m, 3H), 5.62-5.11 (m, 1H), 5.09-4.89 (m, 1H), 4.00 (s, 3H), 3.91 (t, J=6.40 Hz, 2H), 3.28-3.20 (m, 1H), 3.12-3.02 (m, 2H), 2.97-2.72 (m, 5H), 2.64-2.61 (m, 1H), 2.42-2.39 (m, 1H), 2.30-2.20 (m, 2H), 2.07-1.67 (m, 8H), 1.03 (t, J=7.20 Hz, 3H).

### Synthesis D45: Synthesis of N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]propane-2-sulfonamide (Compound 87 and Compound 88)

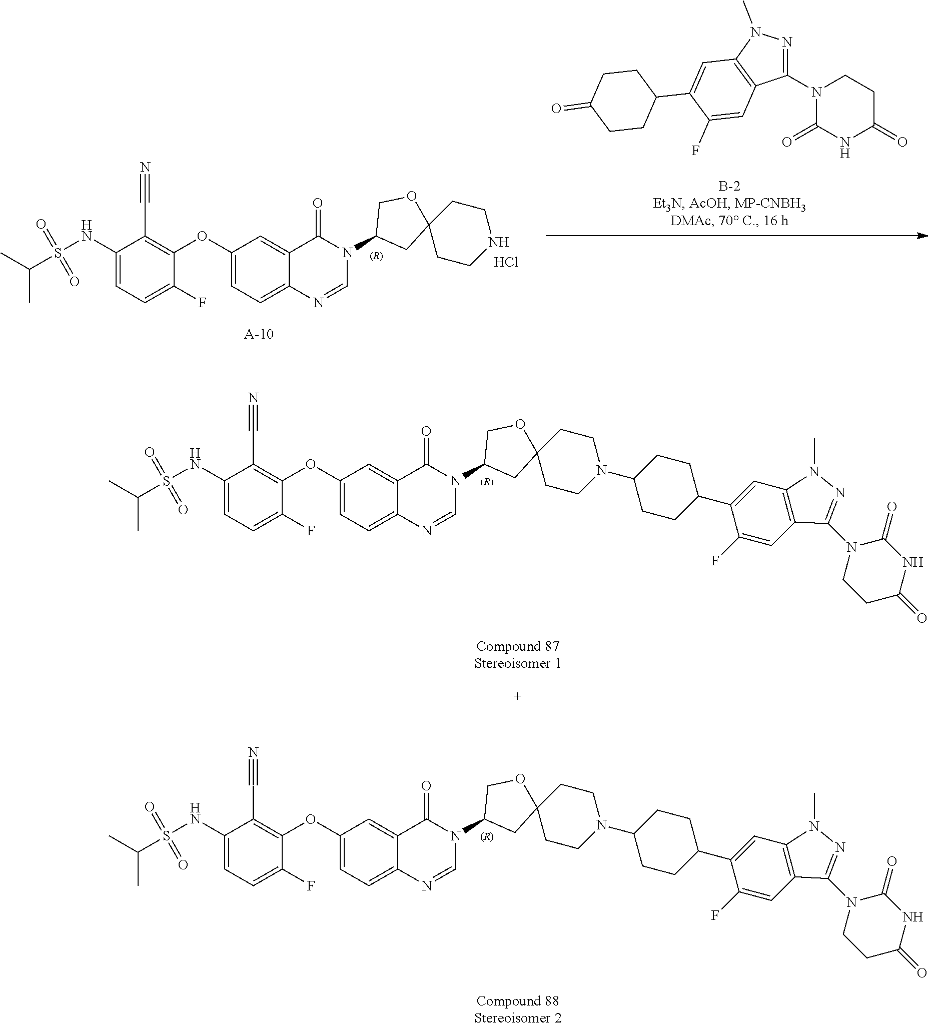

Into a 20 mL pressure vial containing a well-stirred solution of N-[2-cyano-4-fluoro-3-[3-[(3R)-1-oxa-8-azaspiro[4.5]decan-3-yl]-4-oxo-quinazolin-6-yl]oxy-phenyl]propane-2-sulfonamide (A-10) (320 mg, 553.58 mol, HCl salt) in DMAc (10 mL) was added triethylamine (1.66 mmol, 231.47 μL) at room temperature under nitrogen atmosphere and mixture was stirred for 20 min. Reaction mixture was cooled to 0° C. and was added 1-[5-fluoro-1-methyl-6-(4-oxocyclohexyl)indazol-3-yl]hexahydropyrimidine-2,4-dione (B-2) (238.06 mg, 664.30 mol) followed by AcOH (1.11 mmol, 63.32 μL) and resulting mixture was stirred for 3 h at room temperature. Finally, MP-CNBH₃ (600 mg, 553.58 mol) was added, and the reaction mixture was stirred at 70° C. for 16 hours. After completion of reaction, reaction mixture was filtered and filtrate was concentrated under reduced pressure to get a crude residue which was purified by preparative HPLC [Column: X-SELECT CSH (250 mm*19 mm*5 m), Mobile phase A: 10 mM NH₄HCO₃ in milli-Q water; Mobile phase B: acetonitrile].

The first eluting fraction at (Peak-1) was concentrated under reduced pressure at 40° C. and lyophilized to afford Compound 87 (assigned as stereoisomer 1).

Peak-1 (By SFC sequence): N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]propane-2-sulfonamide (Compound 87, 30 mg, 33.76 mol, 6% yield) as a white solid. UPLC-MS (ES+): m/z 884.4 [M+H]⁺. ¹H NMR (400 MHz, DMSO-d₆): δ 10.56 (s, 1H), 8.58 (bs, 1H), 8.33 (s, 1H), 7.77 (d, J=8.80 Hz, 1H), 7.66 (dd, J=2.80, 8.80 Hz, 1H), 7.61-7.48 (m, 2H), 7.47-7.33 (m, 3H), 5.29 (quint, J=6.8 Hz, 1H), 4.22-4.12 (m, 2H), 4.09-3.98 (m, 3H), 3.90 (t, J=6.8 Hz, 3H), 3.69-3.41 (m, 2H), 3.25-3.04 (m, 3H), 2.75 (t, J=6.40 Hz, 2H), 2.22-1.60 (m, 15H), 1.26 (d, J=6.8 Hz, 6H).

Similarly, the second eluted fraction (Peak-2) at t_{R}=2.67 min was concentrated under reduced pressure at 40° C. and lyophilized to afford Compound 88 (assigned as stereoisomer 2).

Peak-2 (by SFC sequence): N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]propane-2-sulfonamide (Compound 88, 22 mg, 24.39 mol, 4% yield) as a white solid. UPLC-MS (ES+): m/z 884.5 [M+H]⁺. ¹H NMR (400 MHz, DMSO-d₆): δ 10.55 (s, 1H), 9.16 (bs, 1H), 8.42-8.18 (m, 1H), 7.76 (dd, J=3.6, 8.8 Hz, 1H), 7.66 (dd, J=3.2, 9.2 Hz, 1H), 7.60-7.10 (m, 5H), 5.29 (quint, J=6.8 Hz, 1H), 4.29-4.10 (m, 2H), 4.00 (s, 3H), 3.90 (t, J=6.8 Hz, 2H), 3.28-2.82 (m, 6H), 2.75 (t, J=6.8 Hz, 2H), 2.49-2.40 (m, 2H), 2.26-1.52 (m, 13H), 1.22 (d, J=6.8 Hz, 6H).

### Synthesis D46: Synthesis of (3S)-3-[6-[2-Cyano-3-[[ethyl(methyl)sulfamoyl]amino]-6-fluoro-anilino]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 89 and Compound 90)

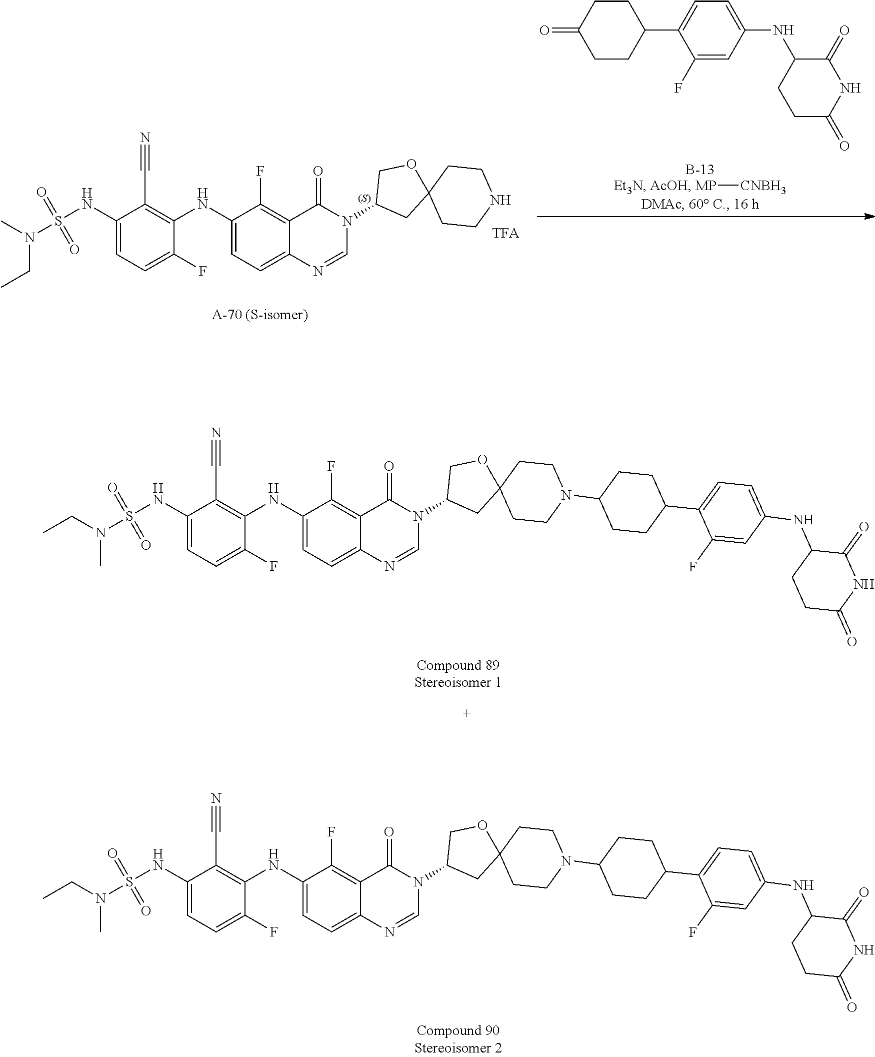

Into a 30 mL pressure vial containing a well stirred solution of 3-[(3S)-8-azaspiro[4.5]decan-3-yl]-6-[2-cyano-3-[[ethyl(methyl)sulfamoyl]amino]-6-fluoro-phenoxy]-4-oxo-quinazoline (A-70) (250.00 mg, 373.88 μmol, TFA Salt) in anhydrous DMAc (3 mL) was added triethylamine (1.87 mmol, 260.56 μL) and stirred for 30 min. To the above mixture, 3-[3-fluoro-4-(4-oxocyclohexyl)anilino]piperidine-2,6-dione (B-13) (119.02 mg, 373.88 μmol) and AcOH (1.12 mmol, 64.21 μL) were added and stirred at room temperature for 3 hours. Subsequently, MP-CNBH₃ (0.400 g, 305.52 μmol) was added and resulting reaction mixture was stirred at 60° C. for 16 hours. After completion of the reaction, the reaction mixture was filtered through cotton pad and filtrate was evaporated under reduced pressure to get a crude residue which was purified by preparative HPLC [Column: X-BRIDGE C8 (250 mm*19 mm*5 μm); Mobile phase A: 10 mM NH₄HCO₃ in water: Mobile phase B: 100% acetonitrile]. The first eluting fraction (Peak-1) was lyophilized to afford Compound 89 (assigned as stereoisomer 1).

Peak-1: (3S)-3-[6-[2-cyano-3-[[ethyl(methyl)sulfamoyl]amino]-6-fluoro-anilino]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro [4.5]decane (Compound 89, 12 mg, 12.90 μmol, 3% yield) as an off-white solid. UPLC-MS (ES+): m/z 876.5 [M+H]⁺. ¹H NMR (400 MHz, DMSO-d₆): δ 10.78 (s, 1H), 8.35 (s, 1H), 8.18 (s, 1H), 7.38 (d, J=8.80 Hz, 1H), 7.34-7.29 (m, 1H), 7.21 (t, J=8.40 Hz, 1H), 7.15 (dd, J=4.40, 9.20 Hz, 1H), 6.97 (t, J=8.40 Hz, 1H), 6.45-6.41 (m, 2H), 5.99 (d, J=7.60 Hz, 1H), 5.32-5.29 (m, 1H), 4.31-4.27 (m, 1H), 4.13-3.99 (m, 2H), 3.07-2.85 (m, 7H), 2.81-2.68 (m, 2H), 2.65-2.57 (m, 4H), 2.41 (t, J=4.40 Hz, 1H), 2.17-1.95 (m, 4H), 1.83-1.58 (m, 7H), 1.60-1.38 (m, 4H), 1.02 (t, J=7.20 Hz, 3H).

Similarly, the second eluted fraction (Peak-2) was lyophilized to afford Compound 90 (assigned as stereoisomer 2).

Peak-2: (3S)-3-[6-[2-cyano-3-[[ethyl(methyl)sulfamoyl]amino]-6-fluoro-anilino]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro [4.5]decane (Compound 90, 15 mg, 16.91 μmol, 4.00% yield, 99% purity) as an off-white solid. UPLC-MS (ES+): m/z 876.5 [M+H]⁺. ¹H NMR (400 MHz, DMSO-d₆): δ 10.75 (s, 1H), 8.39 (s, 1H), 8.17 (s, 1H), 7.39-7.30 (m, 2H), 7.22 (t, J=8.40 Hz, 1H), 7.13 (dd, J=4.40, 9.20 Hz, 1H), 7.06 (t, J=8.80 Hz, 1H), 6.46-6.40 (m, 2H), 5.93 (d, J=8.00 Hz, 1H), 5.29-5.23 (m, 1H), 4.28-4.24 (m, 1H), 4.15-4.09 (m, 3H), 3.02 (m, 2H), 2.99-2.78 (m, 5H), 2.81-2.60 (m, 2H), 2.65-2.57 (m, 4H), 2.45-2.35 (m, 1H), 2.15-1.95 (m, 2H), 1.99-1.72 (m, 1H), 1.72-1.61 (m, 2H), 1.61-1.48 (m, 2H), 1.00 (t, J=7.20 Hz, 3H).

### Synthesis D47: Synthesis of N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]propane-2-sulfonamide (Compound 91 and Compound 92)

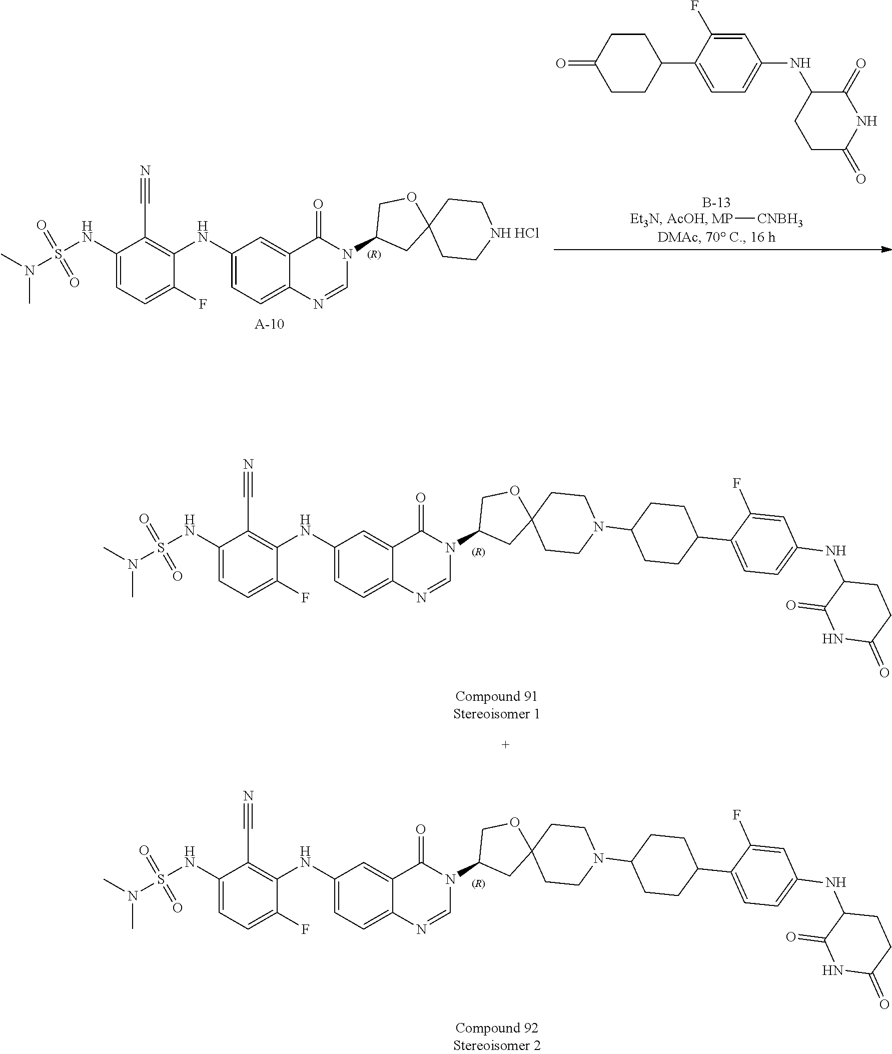

Into a 20 mL pressure vial containing a well-stirred solution of N-[2-cyano-4-fluoro-3-[3-[(3R)-1-oxa-8-azaspiro[4.5]decan-3-yl]-4-oxo-quinazolin-6-yl]oxy-phenyl]propane-2-sulfonamide 1 (A-10) (500 mg, 778.47 mol, HCl salt) in DMAc (10 mL) was added triethylamine (2.34 mmol, 325.51 μL) at room temperature under nitrogen atmosphere and mixture was stirred for 20 min. Reaction mixture was cooled to 0° C. and was added 3-[3-fluoro-4-(4-oxocyclohexyl)anilino]piperidine-2,6-di one 2 (B-13) (300.39 mg, 934.17 mol) followed by AcOH (1.56 mmol, 89.04 μL) and resulting mixture was stirred for 3 h at room temperature. Finally, MP-CNBH₃ (500 mg, 778.47 mol) was added, and the reaction mixture was stirred at 70° C. for 16 hours. After completion of reaction, reaction mixture was filtered and filtrate was concentrated under reduced pressure to get a crude residue which was purified by preparative HPLC [Column. YMC ACTUS C18 (250 mm*20 mm*5 m) and [Mobile phase A: 10 mM NH₄HCO₃ in milli-Q water; Mobile phase B: acetonitrile]. The first eluting fraction was concentrated under reduced pressure at 40° C. and lyophilized to afford Compound 91 (assigned as stereoisomer 1).

Peak-1: N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]propane-2-sulfonamide (Compound 91, 7 mg, 8.25 mol, 1% yield) as a white solid. UPLC-MS (ES+): m/z 844.5 [M+H]⁺. ¹H NMR (400 MHz, DMSO-d₆): δ 10.77 (s, 1H), 9.00 (bs, 1H), 8.31 (s, 1H), 7.76 (d, J=8.8 Hz, 1H), 7.63 (dd, J=3.2, 9.2 Hz, 1H), 7.42-7.23 (m, 3H), 6.97 (t, J=8.8 Hz, 1H), 6.54-6.39 (m, 2H), 6.01 (d, J=7.6 Hz, 1H), 5.38-5.21 (m, 1H), 4.38-4.26 (m, 1H), 4.25-4.06 (m, 2H), 3.20-2.87 (m, 4H), 2.80-2.70 (m, 2H), 2.63-2.54 (m, 3H), 2.22-1.94 (m, 7H), 1.93-1.79 (m, 5H), 1.69-1.45 (m, 4H), 1.24 (d, J=6.8 Hz, 6H).

Similarly, the second eluted fraction was concentrated under reduced pressure at 40° C. and lyophilized to afford Compound 92 (assigned as stereoisomer 2).

Peak-2: N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]propane-2-sulfonamide (Compound 92, 15 mg, 17.54 mol, 2% yield) as a white solid. UPLC-MS (ES+): m/z 844.2 [M+H]⁺. ¹H NMR (400 MHz, DMSO-d₆): δ 10.79 (s, 1H), 8.51 (bs, 1H), 8.32 (s, 1H), 7.77 (d, J=8.8 Hz, 1H), 7.66 (dd, J=2.8, 9.2 Hz, 1H), 7.51 (bs, 1H), 7.37 (d, J=2.8 Hz, 2H), 7.20-7.02 (m, 1H), 6.60-6.37 (m, 2H), 6.03 (d, J=7.6 Hz, 1H), 5.38-5.20 (m, 1H), 4.40-4.28 (m, 1H), 4.21-4.07 (m, 2H), 3.69-3.39 (m, 2H), 3.20-2.89 (m, 3H), 2.80-2.71 (m, 1H), 2.63-2.53 (m, 2H), 2.49-2.36 (m, 2H), 2.30-1.52 (m, 15H), 1.25 (d, J=6.8 Hz, 6H).

### Synthesis D48: Synthesis of N-[2-Cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]cyclopentanesulfonamide (Compound 93 and Compound 94)

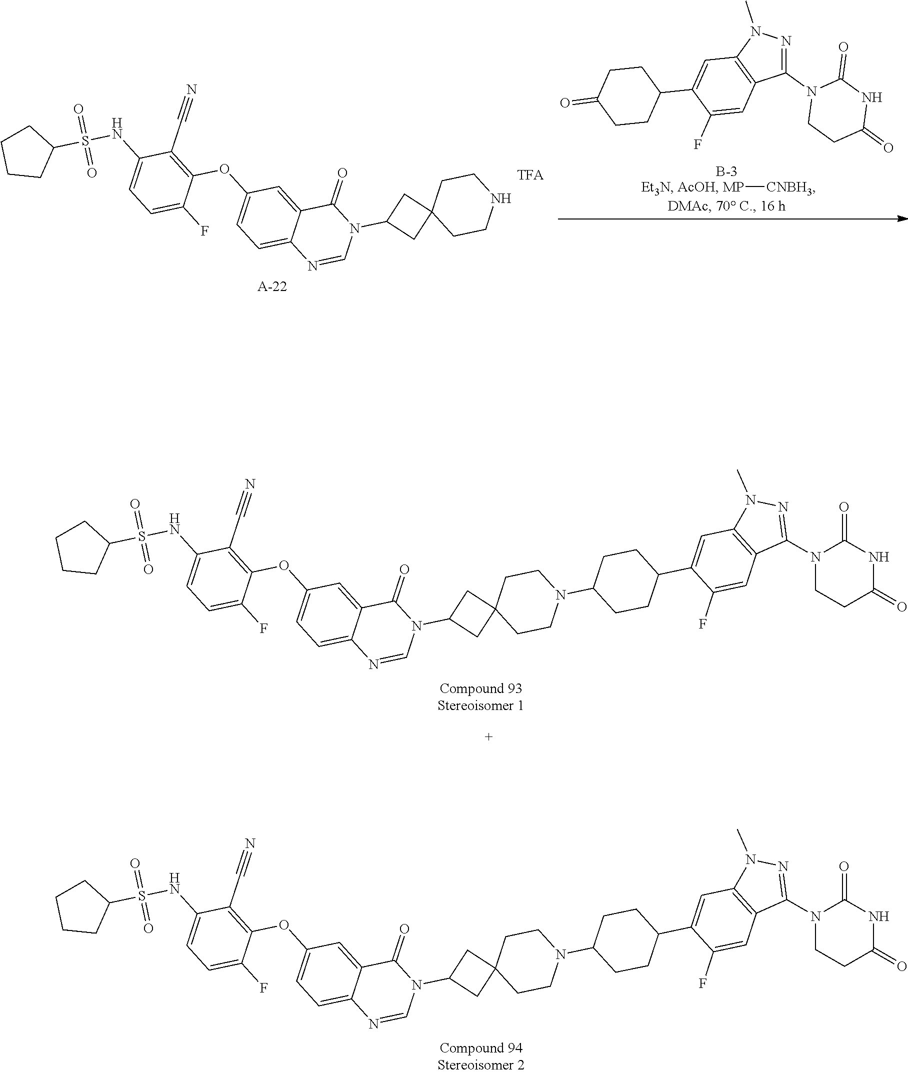

Into a 15 mL pressure vial containing a well-stirred solution of N-[3-[3-(7-azaspiro[3.5]nonan-2-yl)-4-oxo-quinazolin-6-yl]oxy-2-cyano-4-fluoro-phenyl]cyclopentanesulfonamide (A-22) (300 mg, 360.55 μmol, TFA salt) in anhydrous DMAc (5 mL) was added triethylamine (1.80 mmol, 251.26 μL) and stirred at room temperature for 30 minutes. To the above mixture 1-[5-fluoro-1-methyl-6-(4-oxocyclohexyl)indazol-3-yl]hexahydropyrimidine-2,4-dione (B-2) (129.21 mg, 360.55 μmol) and AcOH (1.08 mmol, 61.85 L) were added and stirred for 1 hour. Finally, MP-CNBH₃ (40 mg, 360.55 μmol) was added at room temperature and resulting mixture was stirred at 70° C. for 16 hours. The reaction mixture was filtered through cotton pad and the filtrate was concentrated under reduced pressure to get a crude residue which was purified by reverse phase purification [Column: Redisep R_{f}Gold® reversed-phase C18-teledyne ISCO, 120 g), Mobile phase A: 10 mM NH₄CO₃ in milli-Q water; Mobile phase B: acetonitrile] to get as a mixture of compounds. The mixture was again purified by prep-HPLC [Column: X-Select C18 (250 mm*19 mm*5 μm), Mobile Phase: Mobile phase A: 10 mM NIHCO3 in water, Mobile phase B: 100% acetonitrile]. The first eluting fraction (Peak-1) was lyophilized to afford Compound 93. Peak-1 was assigned as stereoisomer 1.

Peak-1: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]cyclopentanesulfonamide (Compound 93, 15 mg, 16.42 μmol, 5% yield) as an off-white solid. UPLC-MS (ES+): m/z 894.2 [M+H]⁺. ¹H NMR (400 MHz, DMSO-d₆): δ 10.56 (s, 1H), 8.39 (s, 1H), 7.76 (d, J=8.80 Hz, 1H), 7.62 (dd, J=3.20, 9.00 Hz, 1H), 7.54 (d, J=5.60 Hz, 1H), 7.40-7.32 (m, 4H), 5.01-4.93 (m, 1H), 3.99 (s, 3H), 3.90 (t, J=6.80 Hz, 2H), 2.98-2.83 (m, 2H), 2.75 (t, J=6.80 Hz, 2H), 2.47-2.35 (m, 3H), 2.22-2.09 (m, 2H), 2.06-1.98 (m, 3H), 1.95-1.75 (m, 7H), 1.75-1.59 (m, 6H), 1.58-1.41 (m, 2H).

Similarly, the second eluted fraction (Peak-2) was lyophilized to afford Compound 94. Peak-2 was assigned as stereoisomer 2.

Peak-2: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]cyclopentanesulfonamide (Compound 94, 25 mg, 27.79 μmol, 8% yield) as an off-white solid. UPLC-MS (ES+): m/z 894.2 [M+H]⁺. ¹H NMR (400 MHz, DMSO-d₆): δ 10.56 (s, 1H), 8.39 (s, 1H), 7.76 (d, J=8.80 Hz, 1H), 7.63 (dd, J=2.80, 9.00 Hz, 1H), 7.49-7.36 (m, 5H), 4.98-4.92 (m, 1H), 4.03 (s, 3H), 3.91 (t, J=6.80 Hz, 2H), 3.52-3.45 (m, 2H), 2.76 (t, J=6.80 Hz, 2H), 2.43-2.35 (m, 2H), 2.32-2.21 (m, 1H), 2.13-1.63 (m, 17H), 1.58-1.42 (m, 2H).

### Synthesis D49: Synthesis of N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]propane-2-sulfonamide (Compound 95 and Compound 96)

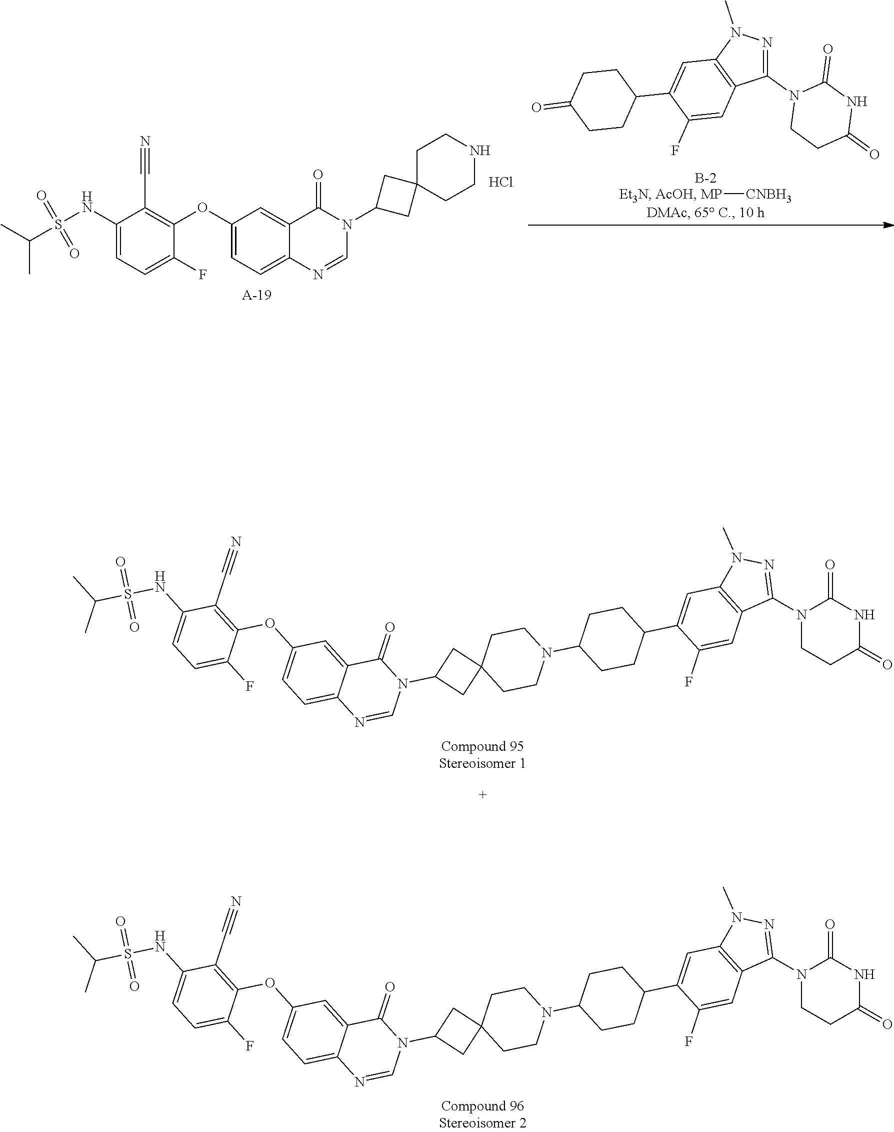

Into a 20 mL pressure vial containing a well-stirred solution of N-[3-[3-(7-azaspiro[3.5]nonan-2-yl)-4-oxo-quinazolin-6-yl]oxy-2-cyano-4-fluoro-phenyl]propane-2-sulfonamide (A-19) (550 mg, 919.84 mol, HCl salt) in DMAc (10 mL) was added triethylamine (2.76 mmol, 384.62 μL) at room temperature under nitrogen atmosphere and mixture was stirred for 5 min. Reaction mixture was cooled to 0° C. and was added 1-[5-fluoro-1-methyl-6-(4-oxocyclohexyl)indazol-3-yl]hexahydropyrimidine-2,4-dione (B-2) (494.46 mg, 1.38 mmol) followed by AcOH (1.84 mmol, 104.22 μL) and resulting mixture was stirred for 2 h at room temperature. Finally, MP-CNBH₃ (550 mg, 919.84 mol) was added, and the reaction mixture was stirred at 65° C. for 10 hours. After the completion of the reaction, the reaction mixture was filtered and filtrate was concentrated under reduced pressure to get a crude residue which was purified by reverse phase column chromatography (Redisep R_{f} Gold®reversed-phase C18-teledyne ISCO, 120 g), [Mobile phase A: 10 mM NH₄HCO₃ in milli-Q water; Mobile phase B: acetonitrile]. The fractions were lyophilized to afford 400 mg of mixture of cis and trans isomers. Further cis and trans isomers were separated by preparative HPLC [Column: X-Bridge C18 (150 mm*19 mm*5 m), Mobile phase A: 10 mM NH₄HCO₃ in milli-Q water; Mobile phase B: acetonitrile]. The first eluting fraction (Peak-1) was concentrated under reduced pressure at 40° C. and lyophilized to afford Compound 95. Peak-1 was assigned as stereoisomer 1.

Peak-1: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]propane-2-sulfonamide (Compound 95, 10 mg, 11.46 mol, 1% yield) as an off-white solid. UPLC-MS (ES+): m/z 868.3 [M+H]⁺. ¹H NMR (400 MHz, DMSO-d₆): δ 10.56 (s, 1H), 8.92 (bs, 1H), 8.38 (s, 1H), 7.75 (d, J=8.8 Hz, 1H), 7.61 (dd, J=3.2, 9.2 Hz, 1H), 7.54 (d, J=6.0 Hz, 1H), 7.49-7.18 (m, 4H), 4.97 (quint, J=8.8 Hz, 1H), 4.00 (s, 3H), 3.91 (t, J=6.4 Hz, 2H), 3.63-3.40 (m, 4H), 2.99-2.88 (m, 3H), 2.75 (t, J=6.40 Hz, 2H), 2.47-2.39 (m, 3H), 2.29-1.41 (m, 13H), 1.21 (d, J=6.4 Hz, 6H).

Similarly, the second eluted fraction (Peak-2) was concentrated under reduced pressure at 40° C. and lyophilized to afford Compound 96. Peak-2 was assigned as stereoisomer 2. (t_{R}=3.09 based on SFC).

Peak-2: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]propane-2-sulfonamide (Compound 96, 35 mg, 39.94 mol, 4% yield) as an off-white solid. UPLC-MS (ES+): m/z 868.5 [M+H]⁺. ¹H NMR (400 MHz, DMSO-d₆): δ 10.54 (s, 1H), 8.53 (bs, 1H), 8.38 (s, 1H), 7.76 (d, J=8.8 Hz, 1H), 7.62 (dd, J=3.2, 9.2 Hz, 1H), 7.48 (d, J=6.0 Hz, 1H), 7.45-7.21 (m, 4H), 4.96 (quint, J=8.8 Hz, 1H), 4.03 (s, 3H), 3.91 (t, J=6.8 Hz, 2H), 3.63-3.33 (m, 2H), 3.20-2.91 (m, 3H), 2.76 (t, J=6.4 Hz, 2H), 2.49-2.25 (m, 6H), 2.13-1.61 (m, 12H), 1.23 (d, J=6.8 Hz, 6H).

### Synthesis D50: Synthesis of (3R)-3-[6-[2-cyano-3-[[ethyl(methyl)sulfamoyl]amino]-6-fluoro-anilino]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 97 and Compound 98)

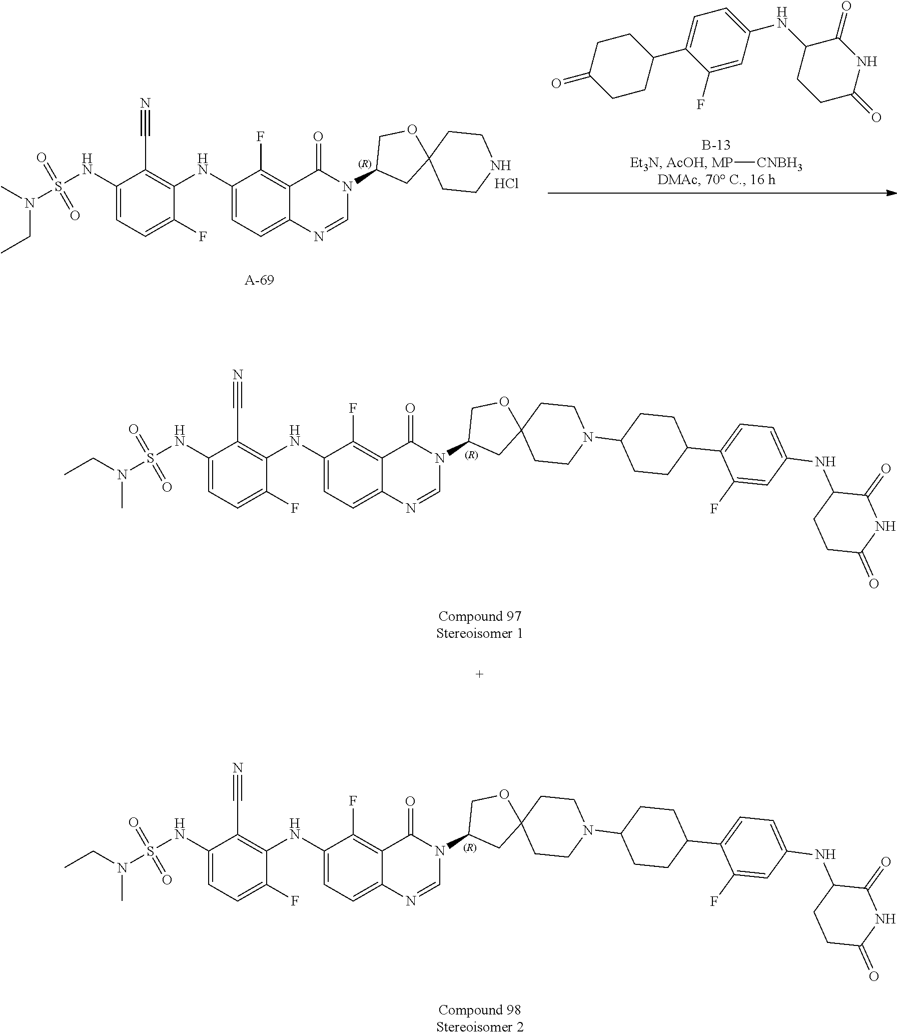

Into a 30 mL pressure vial containing a well-stirred solution of (3R)-3-[6-[2-cyano-3-[[ethyl(methyl)sulfamoyl]amino]-6-fluoro-anilino]-5-fluoro-4-oxo-quinazolin-3-yl]-1-oxa-8-azaspiro[4.5]decane (A-69) (300 mg, 417.98 μmol, HCl salt) in DMAc (10 mL) was added triethylamine (211.48 mg, 2.09 mmol) at room temperature and the resulting mixture was stirred for 30 min at the same temperature. After 30 min, 3-[3-fluoro-4-(4-oxocyclohexyl)anilino]piperidine-2,6-dione (B-13) (133.06 mg, 417.98 μmol) and AcOH (1.25 mmol, 71.78 μL) was added at 0° C. and stirring was continued at room temperature for 2 hours. Subsequently, MP-CNBH₃ (400 mg, 417.98 μmol) was added, and resulting reaction mixture was stirred at 70° C. for 16 hours. After the completion of the reaction, the reaction mixture was filtered through a cotton pad and the filtrate was concentrated under reduced pressure to get a crude residue which was purified by reverse phase column chromatography (Column: Redisep R_{f} Gold® reversed-phase, C18-Teledyne ISCO, 120 g, Mobile phase A: 10 mM ammonium bicarbonate in milli-Q water; Mobile phase B: acetonitrile). Fractions were collected and lyophilized to afford a mixture of cis and trans isomers. Further cis and trans isomers were separated by preparative HPLC (Column: X-Bridge C18(150 mm*19 mm*5 μm), Mobile Phase A: 10 mM ammonium bicarbonate in milli-Q water; Mobile phase B: 100% acetonitrile]. The first eluting fraction (Peak-1) by HPLC was concentrated under reduced pressure at 40° C. and lyophilized to afford Compound 97 (assigned as stereoisomer 1).

Peak-1: (3R)-3-[6-[2-cyano-3-[[ethyl(methyl)sulfamoyl]amino]-6-fluoro-anilino]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 97, 14 mg, 15.45 μmol, 4% yield) as an off-white solid. UPLC-MS (ES+): m/z 876.1 [M+H]⁺. ¹H NMR (400 MHz, DMSO-d₆): δ 10.78 (s, 1H), 8.33 (bs, 1H), 8.29 (s, 1H), 7.40-7.24 (m, 2H), 7.23-7.13 (m, 2H), 6.98 (t, J=8.40 Hz, 1H), 6.45-6.41 (m, 2H), 5.99 (d, J=7.60 Hz, 1H), 5.35-5.25 (m, 1H), 4.35-4.25 (m, 1H), 4.15-4.10 (m, 2H), 3.10-2.85 (m, 7H), 2.84-2.65 (m, 2H), 2.62-2.54 (m, 5H), 2.45-2.35 (m, 1H), 2.13-1.65 (m, 11H), 1.60-1.40 (m, 4H), 1.01 (t, J=7.20 Hz, 3H).

Similarly, the second eluted fraction (Peak-2) by HPLC was concentrated under reduced pressure at 40° C. and lyophilized to afford Compound 98 (assigned as stereoisomer 2).

Peak-2: (3R)-3-[6-[2-cyano-3-[[ethyl(methyl)sulfamoyl]amino]-6-fluoro-anilino]-5-fluoro-4-oxo-quinazolin-3-yl]-8-[4-[4-[(2,6-dioxo-3-piperidyl)amino]-2-fluoro-phenyl]cyclohexyl]-1-oxa-8-azaspiro[4.5]decane (Compound 98, 6 mg, 6.83 μmol, 2% yield) as an off-white solid. UPLC-MS (ES+): m/z 876.6 [M+H]⁺. ¹H NMR (400 MHz, DMSO-d₆): δ 10.76 (s, 1H), 8.46 (bs, 1H), 8.21 (s, 1H), 7.40-7.24 (m, 3H), 7.17 (dd, J=4.0, 9.0 Hz, 1H), 7.04 (t, J=8.40 Hz, 1H), 6.48-6.42 (m, 2H), 5.99 (d, J=7.60 Hz, 1H), 5.33-5.28 (m, 1H), 4.35-4.25 (m, 1H), 4.15-4.10 (m, 2H), 3.15-3.05 (m, 2H), 2.87-2.59 (m, 9H), 2.45-2.36 (m, 1H), 2.10-1.40 (m, 15H), 1.03 (t, J=7.20 Hz, 3H).

### Synthesis D51: Synthesis of (3R)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 99 and Compound 100)

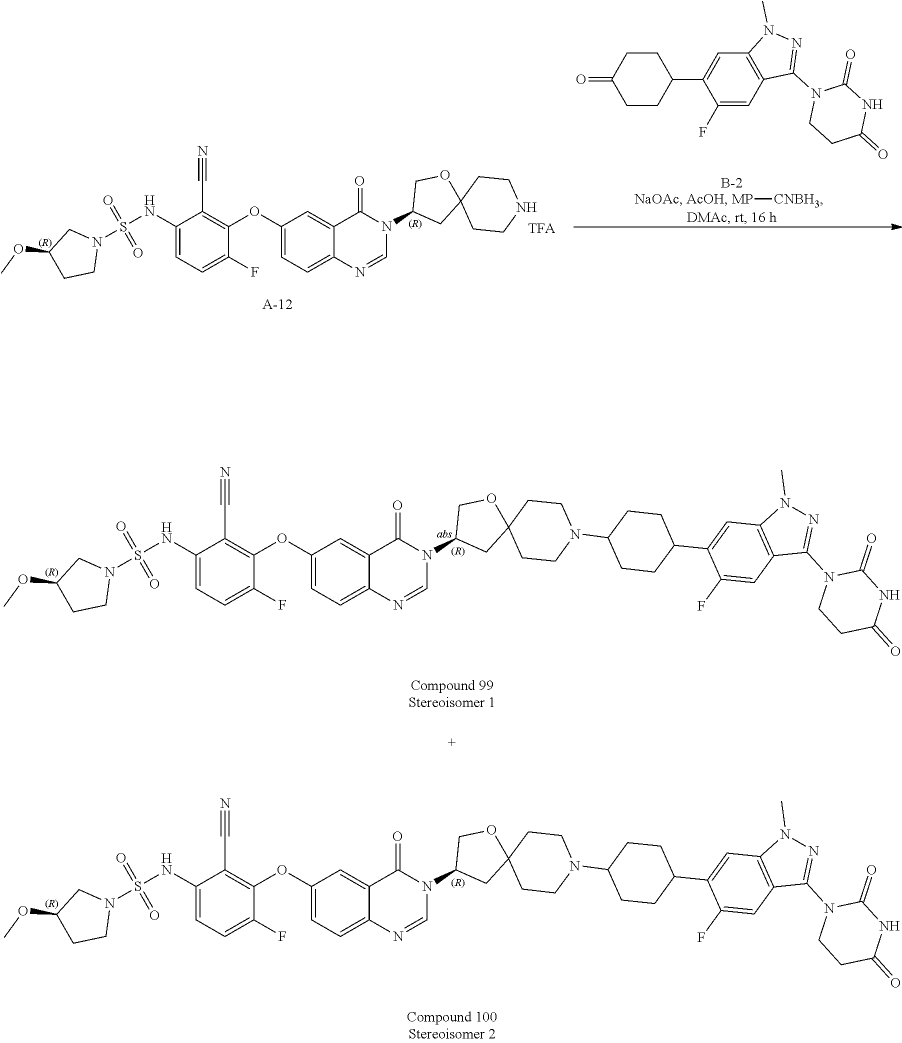

Into a 20 mL pressure vial containing a well-stirred solution of (3R)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-12) (253.32 mg, 280.64 mol, TFA Salt) in DMAc (4 mL) was added anhydrous NaOAc (69.06 mg, 841.87 mol) at room temperature under nitrogen atmosphere and stirred for 20 min. Reaction mixture was cooled to 0° C. and 1-[5-fluoro-1-methyl-6-(4-oxocyclohexyl)indazol-3-yl]hexahydropyrimidine-2,4-dione (B-2) (100.57 mg, 280.64 mol) and AcOH (841.91 mol, 48.15 μL) were added and stirred for 3 hours at room temperature. Finally, MP-CNBH₃ (400 mg, 334.09 mol) was added, and the reaction mixture was stirred at room temperature for 16 hours. After completion of the reaction, the reaction mixture was filtered and the filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fractions were lyophilized to afford 124 mg of mixture of cis and trans isomers. Further cis and trans isomers were separated by preparative HPLC [Column: X-Bridge C18 (250 mm*19 mm*5 m), Mobile phase A: 10 mM NH₄HCO₃ in milli-Q water; Mobile phase B: acetonitrile].

The first eluting fraction (Peak-1) was concentrated under reduced pressure at 40° C. and lyophilized to afford Compound 99 (assigned as stereoisomer 1).

Peak-1: (3R)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 99, 22.59 mg, 23.94 mol, 7% yield) as a white solid. UPLC-MS (ES+): m/z 941.6 [M+H]⁺. ¹H NMR (400 MHz, DMSO-d₆): δ 10.55 (s, 1H), 9.02 (bs, 1H), 8.31 (s, 1H), 7.76 (d, J=9.2 MHz, 1H), 7.64 (dd, J=3.2, 9.2 Hz, 1H), 7.59 (d, J=6.0 MHz, 1H), 7.39-7.31 (m, 4H), 5.32-5.21 (m, 1H), 4.21-4.05 (m, 2H), 3.99 (s, 3H), 3.94-3.81 (m, 3H), 3.29-3.21 (m, 2H), 3.20 (s, 3H), 3.17-3.08 (m, 4H), 3.01-2.94 (m, 2H), 2.89 (bs, 1H), 2.75 (t, J=6.4 Hz, 2H), 2.42 (bs, 1H), 2.28-2.09 (m, 4H), 2.07-1.91 (m, 5H), 1.89-1.51 (m, 7H).

Similarly, the second eluted fraction (Peak-2) was concentrated under reduced pressure at 40° C. and lyophilized to afford Compound 100 (assigned as stereoisomer 2).

Peak-2: (3R)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 100, 29.30 mg, 31.06 mol, 9% yield) as a white solid. UPLC-MS (ES+): m/z 941.6 [M+H]⁺. ¹H NMR (400 MHz, DMSO-d₆): δ 10.55 (s, 1H), 8.57 (bs, 1H), 8.32 (s, 1H), 7.76 (d, J=8.8 MHz, 1H), 7.64 (dd, J=2.8, 8.8 Hz, 1H), 7.55-7.34 (m, 5H), 5.31-5.25 (m, 1H), 4.18-4.08 (m, 2H), 4.02 (s, 3H), 3.93-3.89 (m, 4H), 3.70-3.42 (m, 2H), 3.24-2.90 (m, 8H), 2.75 (t, J=6.8 Hz, 2H), 2.42-2.38 (m, 2H), 2.08-1.80 (m, 16H).

### Synthesis D52: Synthesis of (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-methoxy-pyrrolidine-1-sulfonamide (Compound 101 and Compound 102)

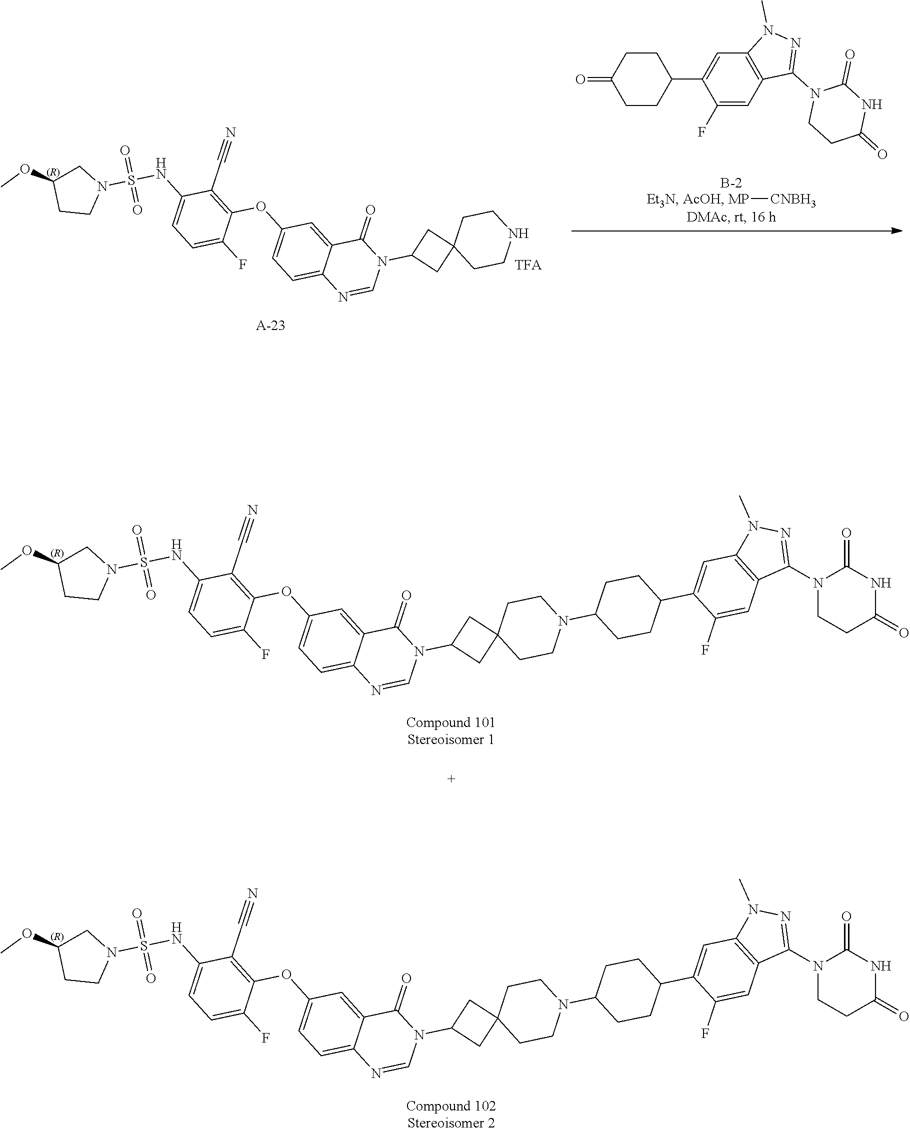

Into a 20 mL pressure vial containing a well-stirred solution of (3R)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-23) (700 mg, 934.45 mol, TFA salt) in DMAc (10 mL) was added triethylamine (2.80 mmol, 390.73 μL) at room temperature under nitrogen atmosphere and mixture was stirred for 5 min. Reaction mixture was cooled to 0° C. and was added 1-[5-fluoro-1-methyl-6-(4-oxocyclohexyl)indazol-3-yl]hexahydropyrimidine-2,4-dione (B-2) (502.31 mg, 1.40 mmol) followed by AcOH (1.87 mmol, 106.88 μL) and resulting mixture was stirred for 2 h at room temperature. Finally, MP-CNBH₃ (700 mg, 934.45 mol) was added, and the reaction mixture was stirred at room temperature for 16 hours. After completion of the reaction, the reaction mixture was filtered and filtrate was concentrated under reduced pressure to get a crude residue which was purified by reverse phase column chromatography (Redisep R_{f} Gold®reversed-phase C18-teledyne ISCO, 120 g), [Mobile phase A: 10 mM NH₄HCO₃ in milli-Q water; Mobile phase B: acetonitrile]. The fractions were lyophilized to afford 550 mg of mixture of cis and trans isomers. Further cis and trans isomers were separated by preparative HPLC [Column: X-SELECT CSH C18 (250 mm*19 mm*5 m), Mobile phase A: 10 mM NH₄HCO₃ in milli-Q water; Mobile phase B: acetonitrile]. The first eluting fraction (Peak-1) was concentrated under reduced pressure at 40° C. and lyophilized to afford Compound 101 (assigned as stereoisomer 1).

Peak-1: (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-methoxy-pyrrolidine-1-sulfonamide (Compound 101, 20 mg, 21.45 mol, 2% yield) as a white solid. UPLC-MS (ES+): m/z 925.6 [M+H]⁺. ¹H NMR (400 MHz, DMSO-d₆): δ 10.53 (s, 1H), 8.99 (bs, 1H), 8.38 (s, 1H), 7.76 (d, J=9.2 Hz, 1H), 7.62 (dd, J=3.2, 8.8 Hz, 1H), 7.53 (d, J=6.0 Hz, 1H), 7.45-7.22 (m, 4H), 5.03-4.89 (m, 1H), 4.00 (s, 3H), 3.95-3.85 (m, 3H), 3.57-3.38 (m, 2H), 3.23-2.81 (m, 12H), 2.76 (t, J=6.4 Hz, 2H), 2.66-2.62 (m, 1H), 2.43-2.37 (m, 2H), 2.22-2.12 (m, 2H), 2.09-1.51 (m, 12H).

Similarly, the second eluted fraction (Peak-2) was concentrated under reduced pressure at 40° C. and lyophilized to afford Compound 102 (assigned as stereoisomer 2).

Peak-2: (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-methoxy-pyrrolidine-1-sulfonamide (Compound 102, 22 mg, 23.30 mol, 3% yield) as a white solid. UPLC-MS (ES+): m/z 925.6 [M+H]⁺. ¹H NMR (400 MHz, DMSO-d₆): δ 10.57 (s, 1H), 8.53 (bs, 1H), 8.39 (s, 1H), 7.76 (d, J=8.8 Hz, 1H), 7.63 (dd, J=2.8, 8.8 Hz, 1H), 7.55 (d, J=6.0 Hz, 1H), 7.43-7.21 (m, 4H), 5.06-4.89 (m, 1H), 4.03 (s, 3H), 4.00-3.84 (m, 4H), 3.60-3.43 (m, 2H), 3.29-3.27 (m, 1H), 3.21-2.72 (m, 12H), 2.44-2.40 (m, 1H), 2.30-1.47 (m, 16H).

### Synthesis D53: Synthesis of (3S)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-methoxy-pyrrolidine-1-sulfonamide (Compound 103 and Compound 104)

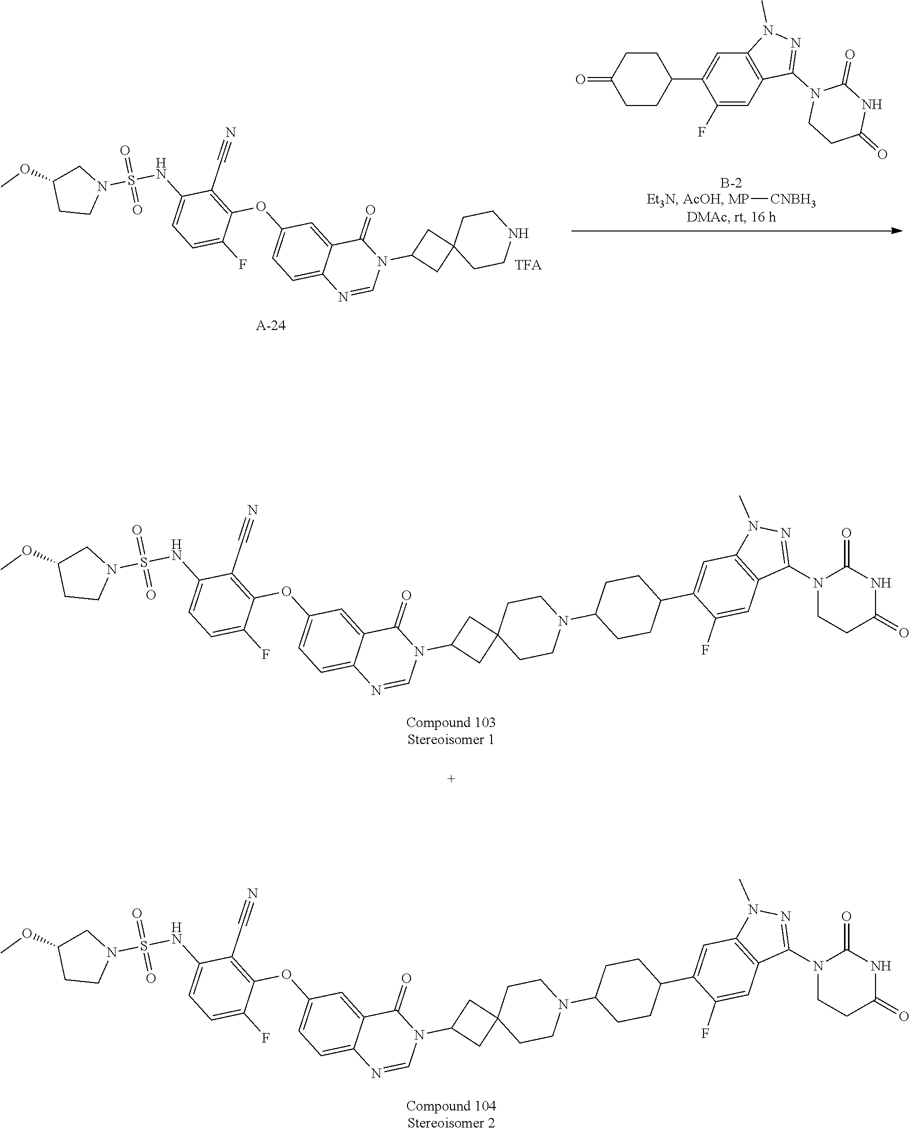

Into a 20 mL pressure vial containing a well-stirred solution of (3S)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-24) (600.00 mg, 749.28 mol, TFA salt) in DMAc (10 mL) was added triethylamine (2.25 mmol, 313.30 μL) at room temperature under nitrogen atmosphere and mixture was stirred for 20 min. Reaction mixture was cooled to 0° C. and was added 1-[5-fluoro-1-methyl-6-(4-oxocyclohexyl)indazol-3-yl]hexahydropyrimidine-2,4-dione (B-2) (402.78 mg, 1.12 mmol) followed by AcOH (1.50 mmol, 86.53 μL) and resulting mixture was stirred for 3 h at room temperature. Finally, MP-CNBH₃ (600 mg, 749.28 mol) was added, and the reaction mixture was stirred at room temperature for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: 10 mM NH₄HCO₃ in milli-Q water; Mobile phase B: acetonitrile]. The fractions were lyophilized to afford 400 mg of mixture of cis and trans isomers. Further cis and trans isomers were separated by preparative HPLC [Column: X-Bridge C18 (250 mm*19 mm*5 m), Mobile phase A: 10 mM NH₄HCO₃ in milli-Q water; Mobile phase B: acetonitrile].

The first eluting fraction (Peak-1 by Prep HPLC) at t_{R}=3.05 min was concentrated under reduced pressure at 40° C. and was lyophilized to afford Compound 103 (assigned as stereoisomer 1), (t_{R}=3.05 based on chiral SFC).

Peak-1: (3S)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-methoxy-pyrrolidine-1-sulfonamide (Compound 103, 33 mg, 35.60 [tmol, 5% yield) as an off-white solid. UPLC-MS (ES+): m/z 925.7 [M+H]⁺. ¹H NMR (400 MHz, DMSO-d₆): δ 10.56 (s, 1H), 9.04 (bs, 1H), 8.39 (s, 1H), 7.76 (d, J=9.20 Hz, 1H), 7.62 (dd, J=3.20, 9.20 Hz, 1H), 7.54 (d, J=6.00 Hz, 1H), 7.43-7.23 (m, 4H), 5.04-4.89 (m, 1H), 4.00 (s, 3H), 3.94-3.89 (m, 3H), 3.29-3.27 (m, 1H), 3.18 (s, 3H), 3.14-2.86 (m, 7H), 2.76 (t, J=6.80 Hz, 2H), 2.45-2.36 (m, 3H), 2.22-2.11 (m, 2H), 2.09-1.57 (m, 14H).

Similarly, the second eluted fraction (Peak-2 by prep HPLC) at t_{R}=2.25 min was concentrated under reduced pressure at 40° C. and was lyophilized to afford Compound 104 (assigned as stereoisomer 2), (t_{R}=2.25 is based on chiral SFC).

Peak-2: (3S)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-methoxy-pyrrolidine-1-sulfonamide (Compound 104, 36 mg, 38.88 mol, 5% yield) as an off-white solid. UPLC-MS (ES+): m/z 925.0 [M+H]⁺. ¹H NMR (400 MHz, DMSO-d₆): δ 10.55 (s, 1H), 8.38 (s, 1H), 7.76 (d, J=8.80 Hz, 1H), 7.62 (dd, J=3.20, 8.80 Hz, 1H), 7.49 (d, J=5.60 Hz, 1H), 7.41-7.34 (m, 4H), 4.95 (quint, J=8.80 Hz, 1H), 4.03 (s, 3H), 3.98-3.88 (m, 5H), 3.22-3.17 (m, 4H), 3.14-2.98 (m, 3H), 2.76 (t, J=2.80 Hz, 3H), 2.21-1.62 (m, 18H).

### Synthesis D54: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]cyclopentanesulfonamide (Compound 105)

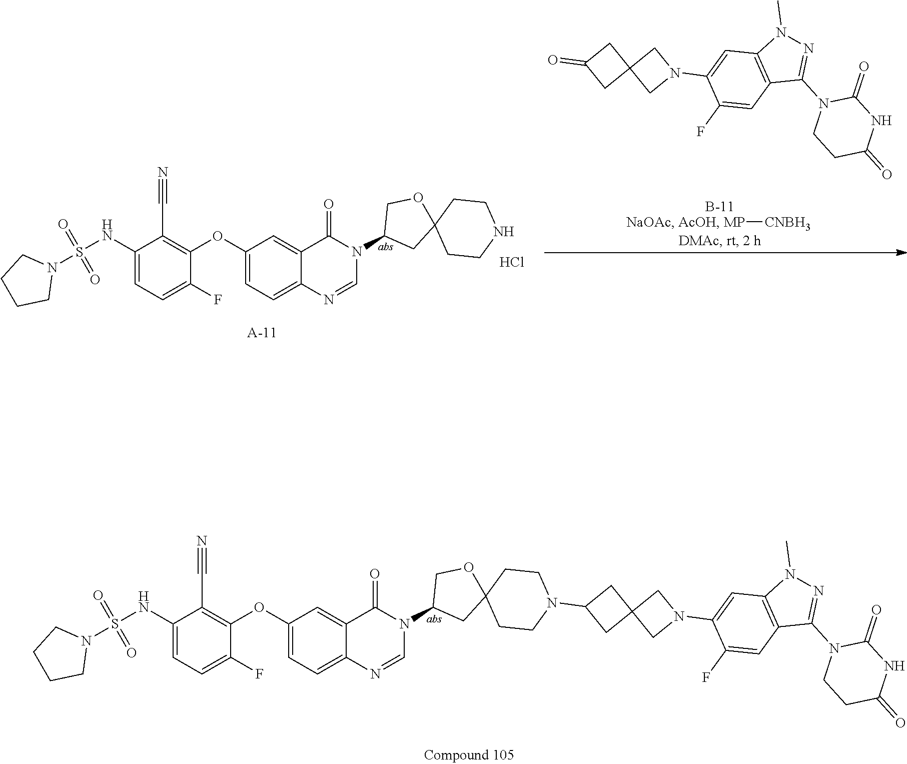

Into a 8 mL glass vial containing a well-stirred solution of 1-[5-fluoro-1-methyl-6-(6-oxo-2-azaspiro[3.3]heptan-2-yl)indazol-3-yl]hexahydropyrimidine-2,4-dione (B-11) (120 mg, 284.36 mol) and N-[2-cyano-4-fluoro-3-[3-[(3R)-1-oxa-8-azaspiro[4.5]decan-3-yl]-4-oxo-quinazolin-6-yl]oxy-phenyl]cyclopentanesulfonamide (A-11) (177.55 mg, 293.91 mol, HCl salt) in anhydrous DMAc (3 mL) was added NaOAc (69.98 mg, 853.07 mol) followed by AcOH (1.42 mmol, 81.31 μL) and stirred for 30 minutes. Subsequently, MP-CNBH₃ (120 mg, 284.36 mol) was added, and the reaction mixture was stirred at room temperature for 2 hours. After completion of the reaction, the reaction mixture was filtered, and filtrate was evaporated under reduced pressure to get a crude residue which was purified by reverse phase by using RediSep R_{f} Gold® 100 g column and [Mobile phase A: 10 mM NH₄HCO₃ in milli-Q water; Mobile phase B: acetonitrile] while the desire product was eluting at 35-45% of the mobile phase. Fraction was lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]cyclopentanesulfonamide (Compound 105, 29 mg, 31.07 mol, 11% yield) as an off-white solid. UPLC-MS (ES+): m/z 923.5 [M+H]⁺. ¹H NMR (400 MHz, DMSO-d₆): δ 10.51 (s, 1H), 9.65 (bs, 1H), 8.32 (s, 1H), 7.77 (d, J=8.80 Hz, 1H), 7.65 (dd, J=3.2, 9.2 Hz, 1H), 7.50 (bs, 1H), 7.37-7.34 (m, 2H), 7.27 (d, J=12.80 Hz, 1H), 6.51 (d, J=7.20 Hz, 1H), δ 5.30-5.27 (m, 1H), 4.18-4.09 (m, 2H), 4.02 (s, 2H), 3.92 (s, 2H), 3.91-3.86 (m, 4H), 3.46-3.37 (m, 1H), 3.13-3.02 (m, 1H), 2.72 (t, J=10.40 Hz, 2H), 2.53-2.51 (m, 1H), 2.46-2.33 (m, 4H), 2.31-2.06 (m, 3H), 1.92-1.48 (m, 13H).

### Synthesis D55: Synthesis of tert-butyl 3-[[5-[[4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (Compound 106 and Compound 107)

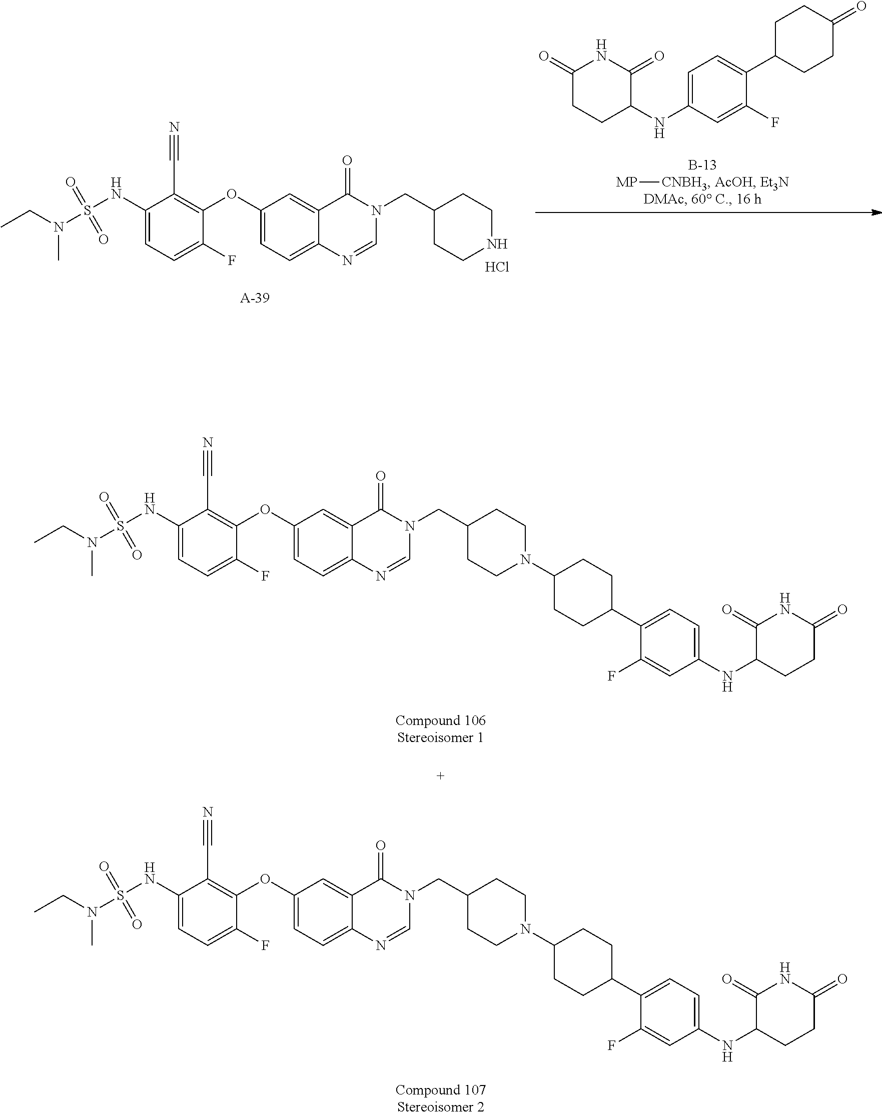

Into 20 mL pressure vial containing a well stirred solution of 6-[2-cyano-3-[[ethyl(methyl)sulfamoyl]amino]-6-fluoro-phenoxy]-4-oxo-3-(4-piperidylmethyl)quinazoline (A-39) (500 mg, 734.98 mol, HCl salt) in DMAc (3 mL) was added triethylamine (2.20 mmol, 307.33 L) at 0° C. and stirred for 30 minutes at room temperature. After 30 minutes, 3-[3-fluoro-4-(4-oxocyclohexyl)anilino]piperidine-2,6-dione (B-13) (280.77 mg, 881.98 mol) and AcOH (3.67 mmol, 206.24 μL) were added at 0° C. and stirred for 2 hours at room temperature. Subsequently, MP-CNBH₃ (500 mg, 734.98 mol) was added at room temperature and resulting reaction mixture was stirred at 60° C. for 16 hours. Reaction mixture was filtered through cotton pad and filtrate was concentrated under reduced pressure to get a crude residue which was purified by reverse phase column chromatography preparative HPLC [Column: X-Bridge C18 (150 mm*19 mm*5 m), Mobile phase A: 10 mm NH₄HCO₃ in milli-Q water; Mobile phase B: 100% acetonitrile, flow rate: 14 mL/min]. First eluting fraction (Peak-1) was collected and lyophilized to afford Compound 106 (assigned as stereoisomer 1).

Peak-1: tert-butyl 3-[[5-[[4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (Compound 106, 100 mg, 0.11 mmol, 18% yield) as an off-white solid. UPLC-MS (ES+): m/z 921.0 [M+H]⁺. ¹H NMR (400 MHz, DMSO-d₆): δ 10.76 (s, 1H), 8.75 (bs, 1H), 8.33 (s, 1H), 7.77 (d, J=8.80 Hz, 1H), 7.64 (dd, J=2.80, 8.80 Hz, 1H), 7.40-7.27 (m, 3H), 6.95 (t, J=8.40 Hz, 1H), 6.45-6.41 (m, 2H), 6.00 (d, J=7.60 Hz, 1H), 4.35-4.25 (m, 1H), 3.97-3.90 (m, 2H), 3.50-3.35 (m, 2H), 3.05-2.95 (m, 3H), 2.82-2.70 (m, 1H), 2.65-2.55 (m, 6H), 2.15-1.96 (m, 4H), 1.91-1.75 (m, 6H), 1.70-1.40 (m, 6H), 1.03 (t, J=7.20 Hz, 3H).

Similarly, the second eluted fraction (Peak-2) was collected and lyophilized to afford Compound 107 (assigned as stereoisomer 2).

Peak-2: tert-butyl 3-[[5-[[4-[3-[5-tert-butoxycarbonyl-4-(2-tert-butoxy-2-oxo-ethoxy)-3-chloro-2-thienyl]anilino]-2,2-dimethyl-1-piperidyl]sulfonylmethyl]-2-fluoro-phenyl]carbamoyl]azetidine-1-carboxylate (Compound 107, 100 mg, 0.107 mmol, 18% yield) as an off-white solid. UPLC-MS (ES+): m/z 919.2 [M−H]⁻. ¹H NMR (400 MHz, DMSO-d₆): δ 10.77 (s, 1H), 8.34 (s, 1H), 7.77 (d, J=8.80 Hz, 1H), 7.64 (dd, J=2.80, 8.80 Hz, 1H), 7.50-7.35 (m, 1H), 7.34 (t, J=2.80 Hz, 1H), 7.31-7.27 (m, 1H), 7.12-7.00 (m, 1H), 6.48-6.43 (m, 2H), 6.01 (d, J=7.60 Hz, 1H), 4.35-4.25 (m, 1H), 4.00-3.90 (m, 2H), 3.60-3.40 (m, 1H), 3.20-2.97 (m, 3H), 2.95-2.82 (m, 2H), 2.81-2.68 (m, 2H), 2.65-2.58 (m, 4H), 2.12-2.03 (m, 2H), 2.00-1.35 (m, 14H), 1.03 (t, J=7.20 Hz, 3H).

### Synthesis D56: Synthesis of (3R)-N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 108 and Compound 109)

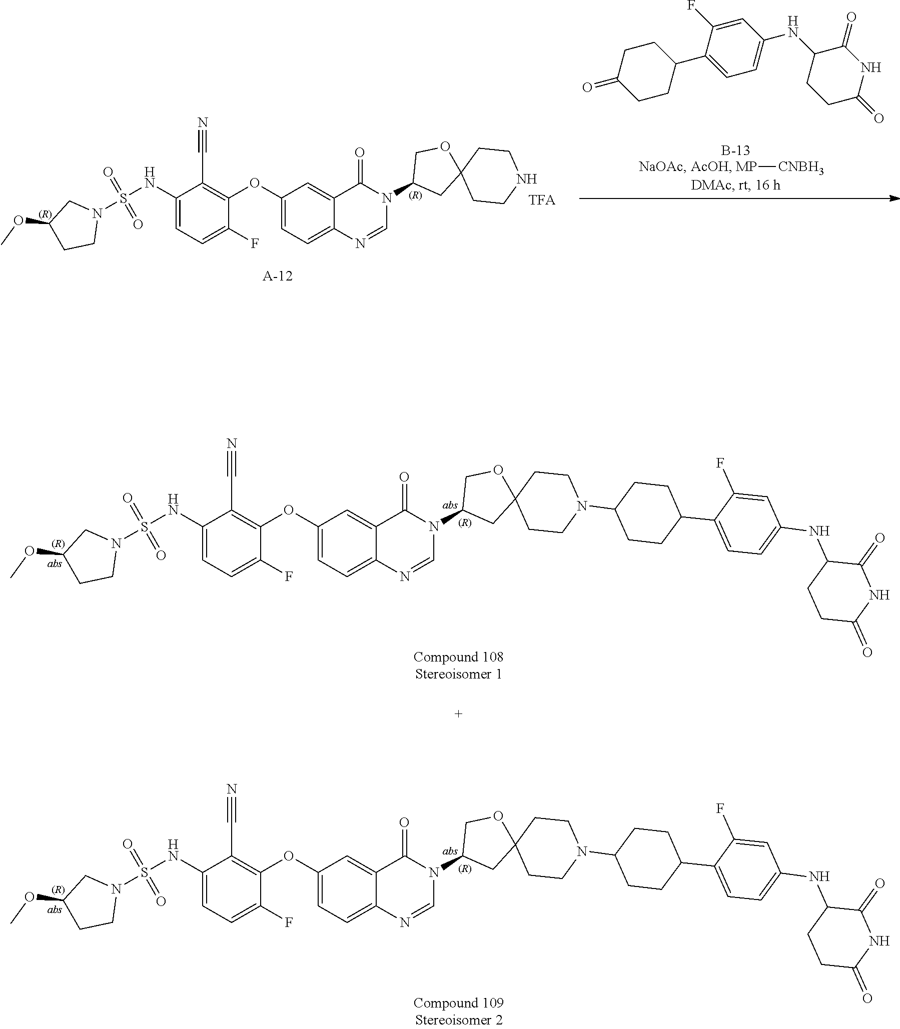

Into a 20 mL pressure vial containing a well-stirred solution of (3R)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide 1 (A-12) (287.53 mg, 318.53 mol, TFA Salt) in DMAc (5 mL) was added anhydrous NaOAc (78.39 mg, 955.59 mol) at room temperature under nitrogen atmosphere and mixture was stirred for 20 min. Reaction mixture was cooled to 0° C. and was added 3-[3-fluoro-4-(4-oxocyclohexyl)anilino]piperidine-2,6-dione 2 (B-13) (101.40 mg, 318.53 mol) followed by AcOH (955.59 mol, 54.64 μL) and resulting mixture was stirred for 3 h at room temperature. Finally, MP-CNBH₃ (460 mg, 318.53 mol) was added, and the reaction mixture was stirred at room temperature for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fractions were lyophilized to afford 210 mg of mixture of cis and trans isomers. Further cis and trans isomers were spearated by preparative HPLC. [Column: X-Bridge C18 (250 mm*19 mm*5 m), Mobile phase A: 10 mM NH₄HCO₃ in milli-Q water; Mobile phase B: acetonitrile].

The first eluting fraction (Peak-1) was concentrated under reduced pressure at 40° C. and lyophilized to afford Compound 108 (assigned as stereoisomer 1).

Peak-1: (3R)-N-[2-cyano-3-[3-[(3R)-8-[4-[4-[(2,6-dioxo-3-piperidyl)amino]-2-fluoro-phenyl]cy clohexyl]-1-oxa-8-azaspiro[4.5]decan-3-yl]-4-oxo-quinazolin-6-yl]oxy-4-fluoro-phenyl]-3-meth oxy-pyrrolidine-1-sulfonamide (Compound 108, 8.33 mg, 9.10 mol, 2% yield) as a white solid. UPLC-MS (ES+): m/z 901.5 [M+H]⁺. ¹H NMR (400 MHz, DMSO-d₆): δ 10.79 (s, 1H), 8.98 (bs, 1H), 8.31 (s, 1H), 7.75 (d, J=8.8 MHz, 1H), 7.64 (dd, J=2.40, 9.20 Hz, 1H), 7.34-7.30 (m, 3H), 6.99-6.95 (m, 1H), 6.46-6.42 (m, 2H), 6.02 (d, J=7.6 MHz, 1H), 5.32-5.22 (m, 1H), 4.35-4.25 (m, 1H), 4.21-4.08 (m, 2H), 3.97-3.88 (m, 1H), 3.32-3.22 (m, 2H), 3.20-2.95 (m, 8H), 2.79-2.65 (m, 1H), 2.62-2.52 (m, 2H), 2.40-2.30 (m, 2H), 2.20-2.02 (m, 5H), 2.00-1.70 (m, 9H), 1.60-1.40 (m, 4H).

Similarly, the second eluted fraction (Peak-2) was concentrated under reduced pressure at 40° C. and lyophilized to afford Compound 109 (assigned as stereoisomer 2).

Peak-2: (3R)-N-[2-cyano-3-[3-[(3R)-8-[4-[4-[(2,6-dioxo-3-piperidyl)amino]-2-fluoro-phenyl]cy clohexyl]-1-oxa-8-azaspiro[4.5]decan-3-yl]-4-oxo-quinazolin-6-yl]oxy-4-fluoro-phenyl]-3-meth oxy-pyrrolidine-1-sulfonamide (Compound 109, 13.25 mg, 14.66 mol, 4% yield) as a white solid. UPLC-MS (ES+): m/z 901.6 [M+H]⁺. ¹H NMR (400 MHz, DMSO-d₆): δ 10.77 (s, 1H), 8.45 (bs, 1H), 8.31 (s, 1H), 7.76 (d, J=8.8 Hz, 1H), 7.64 (dd, J=2.8 Hz, 8.8 Hz, 1H), 7.35-7.34 (m, 3H), 7.09 (bs, 1H), 6.49-6.43 (m, 2H), 6.01 (s, 1H), 5.37-5.23 (m, 1H), 4.36-4.27 (m, 1H), 4.23-4.08 (m, 2H), 3.93 (s, 1H), 3.60-3.41 (m, 2H), 3.18 (s, 3H), 3.12-2.85 (m, 5H), 2.80-2.69 (m, 1H), 2.63-2.55 (m, 2H), 2.42-2.33 (m, 2H), 2.29-2.01 (m, 4H), 2.00-1.55 (m, 14H).

### Synthesis D57: Synthesis of 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]oxy]quinoxaline (Compound 110 and Compound 111)

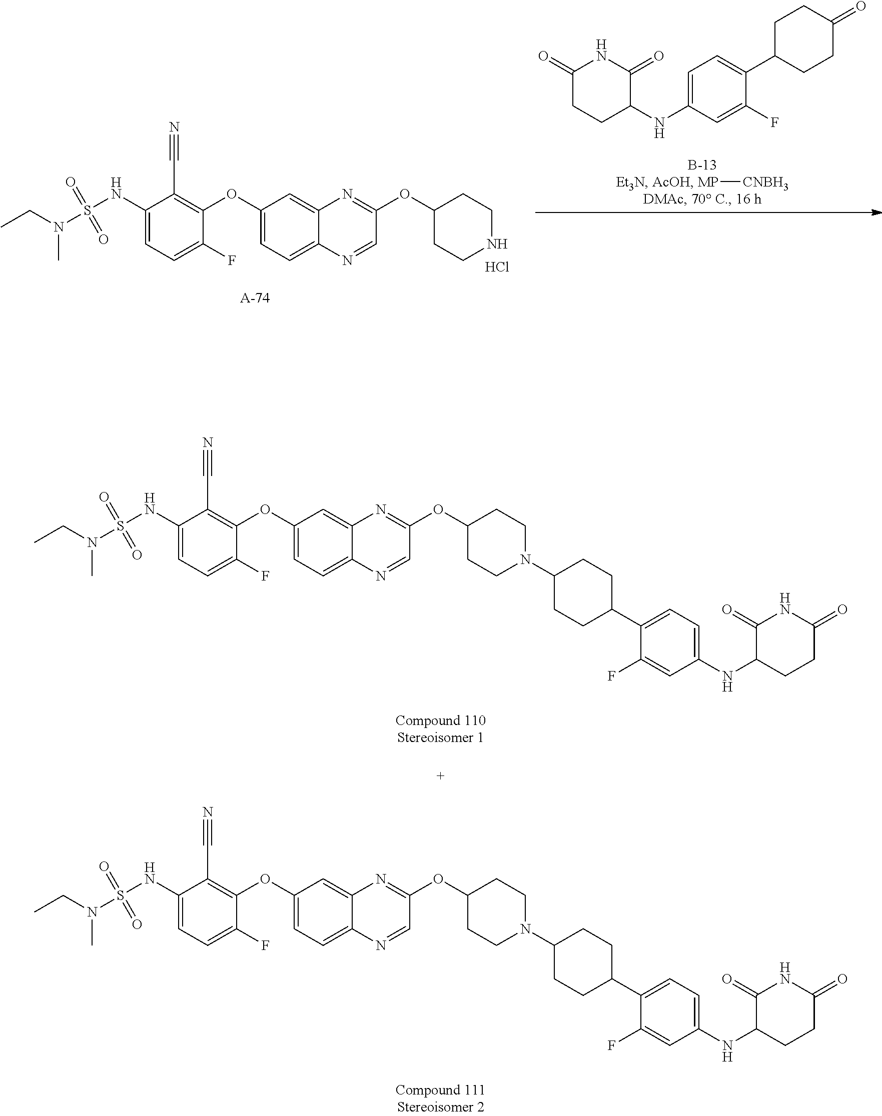

Into a 20 mL pressure vial containing the well stirred solution of 7-[2-cyano-3-[[ethyl(methyl)sulfamoyl]amino]-6-fluoro-phenoxy]-2-(4-piperidyloxy)quinoxaline (A-74) (600 mg, 814.40 mol, HCl salt) in DMAc (10.03 mL) was added triethylamine (4.07 mmol, 567.55 L) at room temperature and stirred for 30 min at the same temperature. To the above mixture, 3-[3-fluoro-4-(4-oxocyclohexyl)anilino]piperidine-2,6-dione (B-13) (259.26 mg, 814.40 mol) and AcOH (2.44 mmol, 139.73 μL) were added at 0° C. and resulting reaction mixture was stirred at room temperature for 3 hours. Subsequently, MP-CNBH₃ (600 mg) was added and stirred at 70° C. for 16 hours. After the completion of the reaction, the reaction mixture was filtered through a cotton pad and filtrate was concentrated under reduced pressure to get a crude residue which was purified by preparative HPLC [Column: X-SELECT CSH (150 mm*19 mm*5 μm), Mobile phase A: 10 mM NH₄HCO₃ in acetonitrile; Mobile phase B: 100% acetonitrile]. Fast-eluting fraction (Peak-1) was lyophilized to afford Compound 110 (assigned as stereoisomer 1).

Peak-1: 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]oxy]quinoxaline (Compound 110, 51.01 mg, 60.09 mol, 7% yield) as an off-white solid. UPLC-MS: m/z 803.1 [M+H]⁺. ¹H NMR (400 MHz, DMSO-d₆): δ 10.79 (bs, 1H), 8.46 (s, 1H), 8.03 (d, J=8.80 Hz, 1H), 7.47 (dd, J=2.80, 9.20 Hz, 1H), 7.33 (t, J=10.00 Hz, 1H), 7.24 (dd, J=4.40, 9.60 Hz, 1H), 7.20-7.01 (m, 1H), 6.98 (t, J=8.40 Hz, 1H), 6.89 (d, J=2.80 Hz, 1H), 6.46-6.42 (m, 2H), 5.99 (d, J=7.60 Hz, 1H), 5.30-5.20 (m, 1H), 4.35-4.25 (m, 1H), 2.98 (d, J=7.20 Hz, 4H), 2.80-2.70 (m, 2H), 2.65-2.59 (m, 2H), 2.57-2.52 (m, 5H), 2.19-2.03 (m, 3H), 2.00-1.70 (m, 7H), 1.52-1.36 (m, 4H), 1.03 (t, J=7.20 Hz, 3H).

Similarly, the second eluted fraction (Peak-2) was lyophilized to afford Compound 111 (assigned as stereoisomer 2).

Peak-2: 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]oxy]quinoxaline (Compound 111, 55.03 mg, 66.07 mol, 8% yield) as an off-white solid. UPLC-MS: m/z 803.1 [M+H]⁺. ¹H NMR (400 MHz, DMSO-d₆): δ 10.79 (s, 1H), 8.47 (s, 1H), 8.03 (d, J=8.80 Hz, 1H), 7.60-7.36 (m, 2H), 7.35-7.25 (m, 1H), 7.22-6.95 (m, 2H), 6.90 (s, 1H), 6.49-6.42 (m, 2H), 6.01 (d, J=7.60 Hz, 1H), 5.50-5.20 (m, 1H), 4.35-4.25 (m, 1H), 3.24-2.95 (m, 4H), 2.94-2.81 (m, 1H), 2.80-2.74 (m, 1H), 2.64-2.55 (m, 4H), 2.46-2.35 (m, 1H), 2.30-2.05 (m, 4H), 2.03-1.40 (m, 12H), 1.03 (t, J=7.20 Hz, 3H).

### Synthesis D58: Synthesis of N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]cyclopentanesulfonamide (Compound 112 and Compound 113)

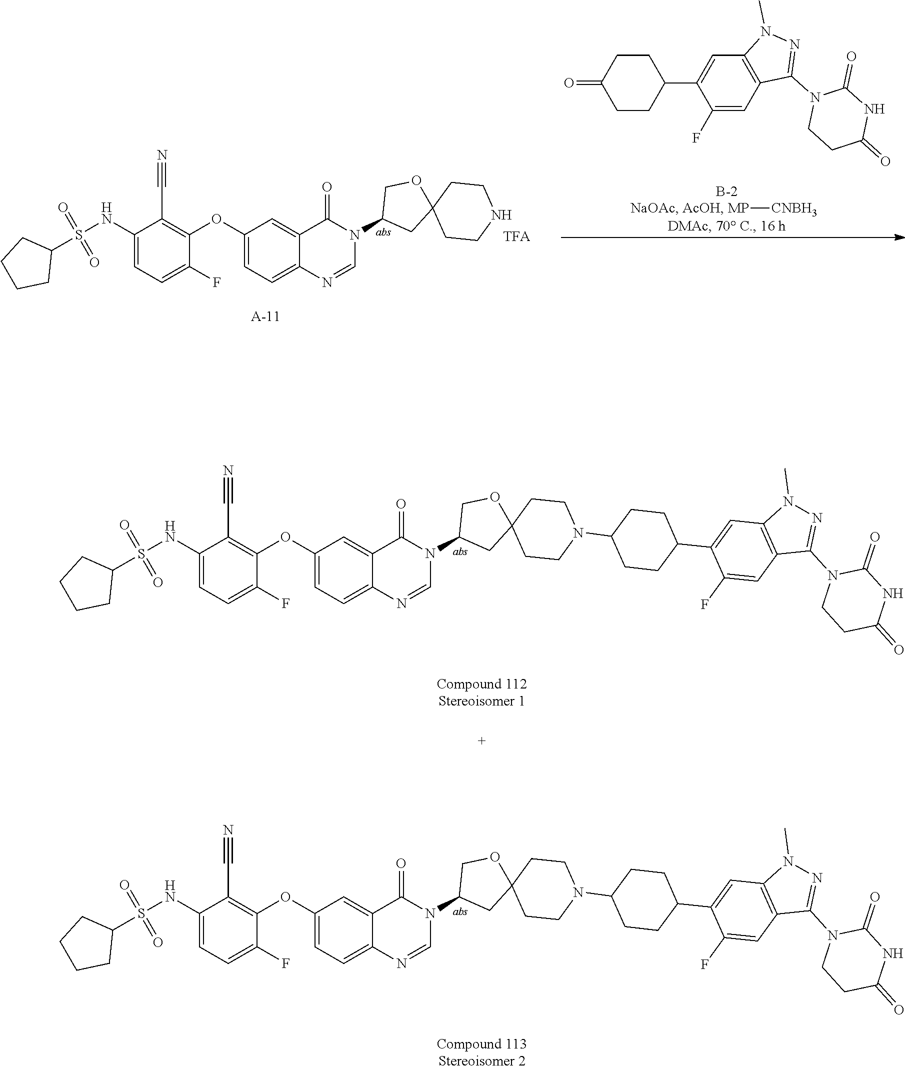

Into a 20 mL pressure vial containing a well-stirred solution of N-[2-cyano-4-fluoro-3-[3-[(3R)-1-oxa-8-azaspiro[4.5]decan-3-yl]-4-oxo-quinazolin-6-yl]oxy-phenyl]cyclopentanesulfonamide (A-11) (280.00 mg, 357.37 mol, TFA salt) in DMAc (5 mL) was added anhydrous NaOAc (87.95 mg, 1.07 mmol) at room temperature under nitrogen atmosphere and mixture was stirred for 20 min. Reaction mixture was cooled to 0° C. and was added 1-[5-fluoro-1-methyl-6-(4-oxocyclohexyl)indazol-3-yl]hexahydropyrimidine-2,4-dione (B-2) (153.68 mg, 428.84 mol) followed by AcOH (107.30 mg, 1.79 mmol) and resulting mixture was stirred for 3 h at room temperature. Finally, MP-CNBH₃ (280 mg, 357.37 mol) was added, and the reaction mixture was stirred at 70° C. for 16 hours. After the completion of the reaction, the reaction mixture was filtered and filtrate was concentrated under reduced pressure to get a crude residue which was purified by reverse phase column chromatography (Redisep R_{f} Gold®reversed-phase C18-teledyne ISCO, 120 g), [Mobile phase A: NH₄HCO₃ in milli-Q water; Mobile phase B: acetonitrile]. Fractions were lyophilized to afford 200 mg of mixture of cis and trans isomers. Further cis and trans isomers were separated by preparative HPLC [Column: X-Bridge C8 (250 mm*19 mm*5 m), Mobile phase A: 10 mM NH₄HCO₃ in milli-Q water; Mobile phase B: acetonitrile]. The first eluting fraction (peak-1) was concentrated under reduced pressure at 40° C. and lyophilized to afford Compound 112 (assigned as stereoisomer 1).

Peak-1: N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]cyclopentanesulfonamide (Compound 112, 7.5 mg, 8.18 mol, 2% yield) as an off-white solid. UPLC-MS (ES+): m/z 909.9 [M+H]⁺. ¹H NMR (400 MHz, DMSO-d₆): δ 10.56 (s, 1H), 9.06 (s, 1H), 8.32 (s, 1H), 7.76 (d, J=8.80 Hz, 1H), 7.66-7.63 (m, 1H), 7.54 (d, J=5.60 Hz, 1H), 7.40-7.21 (m, 4H), 5.37-5.20 (m, 1H), 4.29-4.12 (m, 2H), 3.99 (s, 3H), 3.92-3.89 (m, 2H), 3.17-2.88 (m, 3H), 2.75 (t, J=6.40 Hz, 2H), 2.44-2.38 (m, 1H), 2.24-2.18 (m, 25H).

Similarly, the second eluted fraction (peak-2) was concentrated under reduced pressure at 40° C. and lyophilized to afford Compound 113 (assigned as stereoisomer 2).

Peak-2: N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]cyclopentanesulfonamide (Compound 113, 15 mg, 16.15 mol, 5% yield) as an off-white solid. UPLC-MS (ES+): m/z 910.2 [M+H]⁺. ¹H NMR (400 MHz, DMSO-d₆): δ 10.56 (s, 1H), 8.64 (bs, 1H), 8.33 (s, 1H), 7.76 (d, J=8.80 Hz, 1H), 7.71-7.59 (m, 1H), 7.60-7.20 (m, 5H), 5.35-5.20 (m, 1H), 4.23-4.03 (m, 2H), 4.02 (s, 3H), 3.99-3.81 (m, 2H), 3.80-3.39 (m, 4H), 3.29-2.90 (m, 4H), 2.76 (t, J=6.80 Hz, 2H), 2.43-2.40 (m, 1H), 2.28-1.43 (m, 20H).

### Synthesis D59: Synthesis of (3S)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 114 and Compound 115)

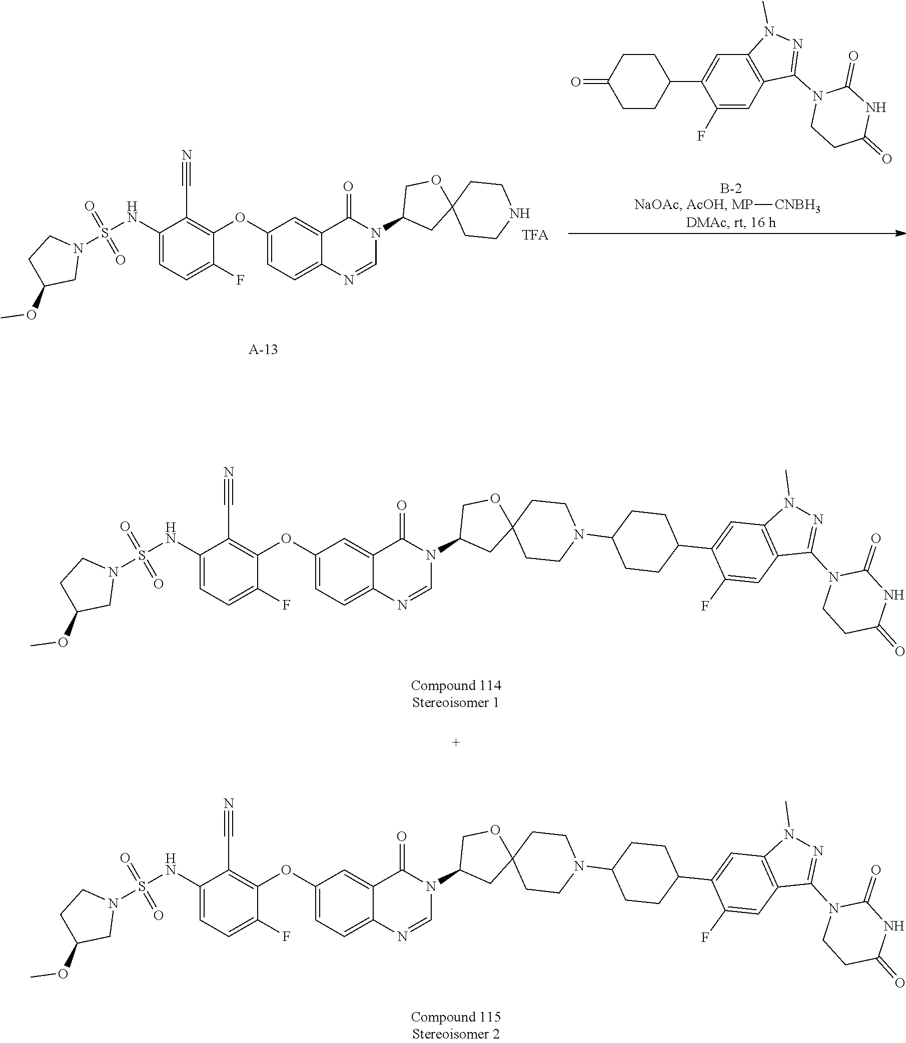

Into a 20 mL pressure vial containing a well-stirred solution of (3S)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-13) (225 mg, 315.72 mol, TFA salt) in DMAc (2.98 mL) was added anhydrous NaOAc (129.49 mg, 1.58 mmol) at room temperature and resulting mixture was stirred for 30 min at the same temperature. After 30 min, 1-[5-fluoro-1-methyl-6-(4-oxocyclohexyl)indazol-3-yl]hexahydropyrimidine-2,4-dione (B-2) (169.71 mg, 473.57 mol) and AcOH (1.58 mmol, 90.27 μL) were added at 0° C. and mixture was stirred for 3 hours at room temperature. Subsequently, MP-CNBH₃ (250 mg, 315.72 mol) was added, and the reaction mixture was stirred for 16 h at room temperature. After the completion of the reaction, the reaction mixture was filtered through cotton pad and the filtrate was concentrated under reduced pressure to get a crude residue which was purified by reverse phase (Column: Redisep R_{f} Gold® reversed-phase, C18-Teledyne ISCO, 120 g), [Mobile phase A: 10 mM NH₄HCO₃ in milli-Q water; Mobile phase B: acetonitrile]. Fractions were collected and lyophilized to afford 100 mg of a mixture of cis and trans isomers. Further cis and trans isomers were separated by preparative HPLC [Column: X-Bridge C18 (250 mm*19 mm*5 m), Mobile phase A: 10 mM NH₄OH (pH-10); Mobile phase B: 100% acetonitrile, flow rate: 14 mL/min]. The first eluting fraction (Peak-1) was concentrated under reduced pressure and lyophilized to afford Compound 114 (assigned as stereoisomer 1).

Peak-1: (3S)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (21.77 mg, 22.69 mol, 7% yield) as a white solid. UPLC-MS (ES+): m/z 941.7 [M+H]⁺. ¹H NMR (400 MHz, DMSO-d₆): δ 10.56 (s, 1H), 9.04 (bs, 1H), 8.32 (s, 1H), 7.76 (d, J=8.80 Hz, 1H), 7.65 (dd, J=2.80, 8.80 Hz, 1H), 7.54 (d, J=6.00 Hz, 1H), 7.40-7.32 (m, 4H), 5.33-5.27 (m, 1H), 4.20-4.11 (m, 2H), 4.00 (s, 3H), 3.96-3.88 (m, 3H), 3.24 (t, J=4.80 Hz, 2H), 3.18 (s, 3H), 3.11-2.85 (m, 6H), 2.76 (t, J=6.80 Hz, 2H), 2.43-2.35 (m, 2H), 2.22-1.92 (m, 9H), 1.83-1.68 (m, 7H).

Similarly, the second eluted fraction (Peak-2) was concentrated under reduced pressure at 40° C. and lyophilized to afford Compound 115 (assigned as stereoisomer 2).

Peak-2: (3S)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (26.02 mg, 27.61 mol, 9% yield) as a white solid. UPLC-MS (ES+): m/z 941.6 [M+H]⁺. ¹H NMR (400 MHz, DMSO-d₆): δ 10.56 (s, 1H), 8.61 (bs, 1H), 8.33 (s, 1H), 7.76 (d, J=8.80 Hz, 1H), 7.65 (dd, J=2.80, 8.80 Hz, 1H), 7.48-7.34 (m, 5H), 5.36-5.29 (m, 1H), 4.18-4.13 (m, 2H), 4.03 (s, 3H), 3.93-3.90 (m, 3H), 3.61-3.52 (m, 2H), 3.18 (s, 3H), 3.12-2.95 (s, 5H), 2.79-2.74 (m, 3H), 2.43-2.38 (m, 2H), 2.26-2.15 (m, 1H), 2.12-1.98 (m, 5H), 1.97-1.94 (m, 1H), 1.92-1.70 (m, 5H).

### Synthesis D60: Synthesis of 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]methoxy]quinoxaline (Compound 116 and Compound 117)

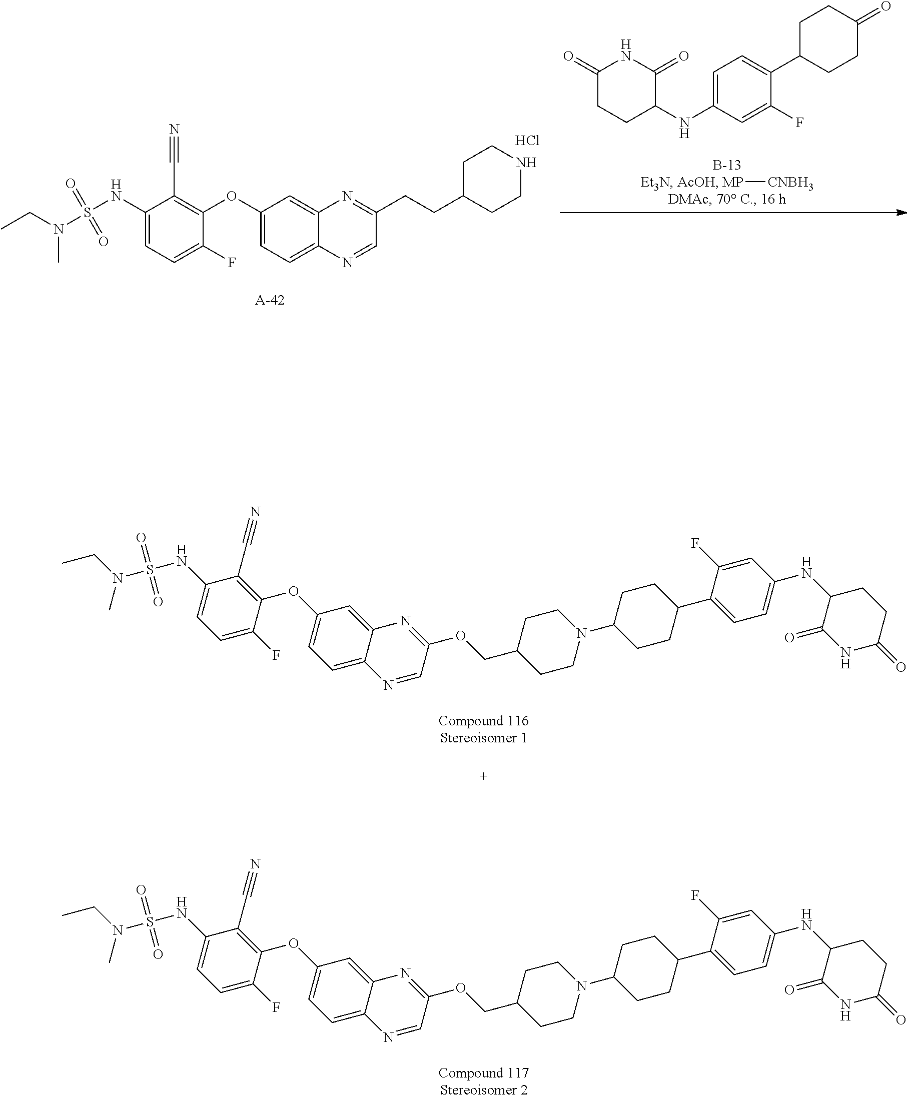

Into a 40 mL pressure vial containing a well-stirred solution of 7-[2-cyano-3-[[ethyl(methyl)sulfamoyl]amino]-6-fluoro-phenoxy]-2-(4-piperidylmethoxy)quinoxaline (A-42) (600 mg, 1.03 mmol, HCl salt) in DMAc (8.0 mL) was added triethylamine (5.17 mmol, 720.89 μL) at room temperature and mixture was stirred for 30 min. To the above mixture, 3-[3-fluoro-4-(4-oxocyclohexyl)anilino]piperidine-2,6-dione (B-13) (329.30 mg, 1.03 mmol) and AcOH (3.10 mmol, 177.65 μL) were added at 0° C. and stirring was continued at room temperature for 2 hours. Finally, MP-CNBH₃ (1 g, 1.03 mmol) was added, and resulting reaction mixture was stirred at 70° C. for 16 hours. After the completion of the reaction, the reaction mixture was filtered through cotton pad and the filtrate was concentrated under reduced pressure to get a crude residue which was purified by reverse phase column chromatography (Column: Redisep R_{f} Gold® reversed-phase, C18-Teledyne ISCO, 120 g, Mobile phase A: 10 mM ammonium bicarbonate in milli-Q water; Mobile phase B: acetonitrile). Fractions were collected and lyophilized to afford mixture of cis and trans isomers. Further cis and trans isomers were separated by preparative HPLC (Column: X-Bridge C18(150 mm*19 mm*5 μm), Mobile Phase A: 10 mM ammonium bicarbonate in milli-Q water; Mobile phase B: 100% acetonitrile). The first eluting fraction (Peak-1) was concentrated under reduced pressure at 40° C. and lyophilized to afford Compound 116 (assigned as stereoisomer 1).

Peak-1: 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]methoxy]quinoxaline (Compound 116.33 mg, 38.50 μmol, 4% yield) as an off-white solid. UPLC-MS (ES+): m/z 816.6 [M+H]⁺. ¹H NMR (400 MHz, DMSO-d₆): δ 10.79 (s, 1H), 8.88 (bs, 1H), 8.50 (s, 1H), 8.05 (d, J=9.20 Hz, 1H), 7.50 (dd, J=2.40, 9.00 Hz, 1H), 7.40-7.20 (m, 2H), 6.99-6.89 (m, 2H), 6.46-6.40 (m, 2H), 6.02 (d, J=7.60 Hz, 1H), 4.38-4.27 (m, 3H), 3.10-2.98 (m, 3H), 2.60-2.54 (m, 4H), 2.11-1.80 (m, 9H), 1.70-1.45 (m, 6H), 1.03 (t, J=7.20 Hz, 3H).

Similarly, the second eluted fraction (Peak-2) was concentrated under reduced pressure at 40° C. and lyophilized to afford Compound 117 (assigned as stereoisomer 2).

Peak-2: 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]methoxy]quinoxaline (Compound 117), 41 mg, 49.58 μmol, 5% yield) as an off-white solid. UPLC-MS (ES+): m/z 816.6 [M+H]⁺. ¹H NMR (400 MHz, DMSO-d₆): δ 10.77 (s, 1H), 8.50 (s, 1H), 8.05 (d, J=9.20 Hz, 1H), 7.50 (d, J=7.60 Hz, 1H), 7.37-7.30 (m, 1H), 7.10-7.06 (m, 1H), 6.92 (s, 1H), 6.50-6.02 (m, 4H), 4.48-4.26 (m, 3H), 3.70-3.55 (m, 1H), 3.10-2.70 (m, 6H), 2.61-2.58 (m, 2H), 2.19-1.50 (m, 16H), 1.07 (t, J=7.20 Hz, 3H).

### Synthesis D61: Synthesis of 7-[2-Cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]methoxy]quinoxaline (Compound 118 and Compound 119)

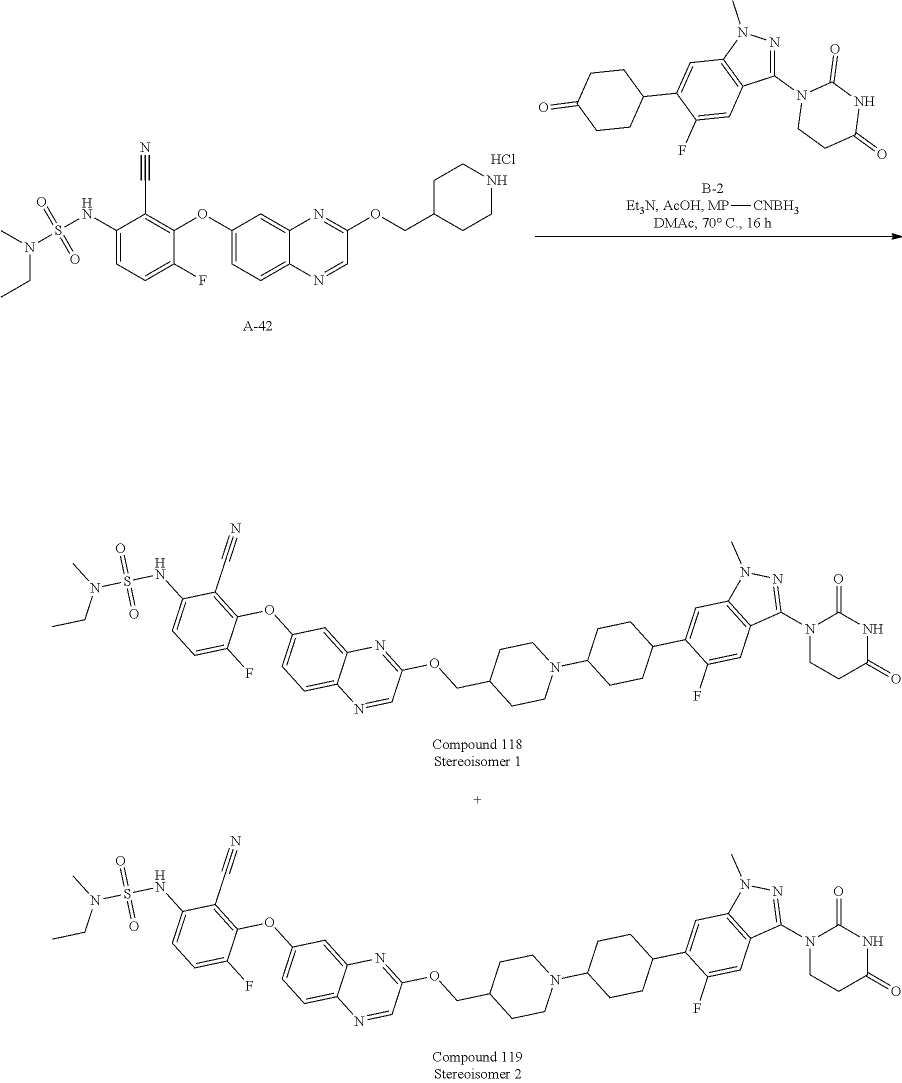

Into a 20 mL pressure capped vial containing a well stirred solution of 7-[2-cyano-3-[[ethyl(methyl)sulfamoyl]amino]-6-fluoro-phenoxy]-2-(4-piperidylmethoxy)quinoxaline (A-42) (600 mg, 1.03 mmol, HCl salt) in anhydrous DMAc (10.06 mL) was added triethyl amine (5.17 mmol, 720.89 μL) and stirred for 30 minutes. To the above mixture 1-[5-fluoro-1-methyl-6-(4-oxocyclohexyl)indazol-3-yl]hexahydropyrimidine-2,4-dione (B-2) (370.70 mg, 1.03 mmol) and AcOH (3.10 mmol, 177.47 μL) were added and stirred for 1 hour. Subsequently, MP-CNBH₃ (1000 mg, 39.96 mol) was added and stirred at 70° C. for 16 hours. After completion of the reaction, the reaction mixture was filtered through cotton pad and the filtrate was concentrated under reduced pressure to get a crude reaction which was purified by preparative HPLC [Column: X-BRIDGE C8 (250*19*5 u; Mobile phase A: 10 mM NH₄HCO₃ in milli-Q water, Mobile Phase B: 100% acetonitrile] to get a mixture of cis and trans isomers. Mixture was again purified by preparative-HPLC using the method [Column: X-BRIDGE C8 (250 mm*19 mm*5 μm): Mobile phase A: 10 mM NH₄CO₃ in H₂O, Mobile phase B: 100% acetonitrile]. The first eluting fraction (Peak-1) was lyophilized to afford Compound 118 (assigned as stereoisomer 1).

Peak-1: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]methoxy]quinoxaline (Compound 118, 45 mg, 52.01 Pmol, 5% yield) as an off-white solid. UPLC-MS (ES+): m/z 857.5 [M+H]⁺. ¹H NMR (400 MHz, DMSO-d₆): δ 10.56 (s, 1H), 8.97 (bs, 1H), 8.50 (s, 1H), 8.05 (dd, J=5.20, 9.00 Hz, 1H), 7.54-7.25 (m, 5H), 6.91 (s, 1H), 4.37-4.35 (m, 2H), 3.99 (s, 3H), 3.91 (t, J=6.80 Hz, 2H), 3.51-3.47 (m, 1H), 3.20-2.83 (m, 6H), 2.76 (t, J=6.80 Hz, 2H), 2.59 (s, 3H), 2.15-1.92 (m, 8H), 1.76-1.52 (m, 6H), 1.04 (t, J=7.20 Hz, 3H).

Similarly, the second eluted fraction was lyophilized to afford Compound 119 (assigned as stereoisomer 2).

Peak-2: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]methoxy]quinoxaline (Compound 119, 85 mg, 96.90 Pmol, 9% yield) as an off-white solid. UPLC-MS (ES+): m/z 857.5 [M+H]⁺. ¹H NMR (400 MHz, DMSO-d₆): δ 10.56 (s, 1H), 8.62 (bs, 1H), 8.51 (s, 1H), 8.06 (d, J=9.20 Hz, 1H), 7.51-7.29 (m, 5H), 6.91 (s, 1H), 4.45-4.18 (m, 2H), 4.03 (s, 3H), 3.91 (t, J=6.40 Hz, 2H), 3.71-3.55 (m, 1H), 3.17-2.95 (m, 5H), 2.76 (t, J=6.80 Hz, 2H), 2.59 (s, 3H), 2.25-1.43 (m, 15H), 1.03 (t, J=7.20 Hz, 3H).

### Synthesis D62: Synthesis of (3R)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 120 and Compound 121)

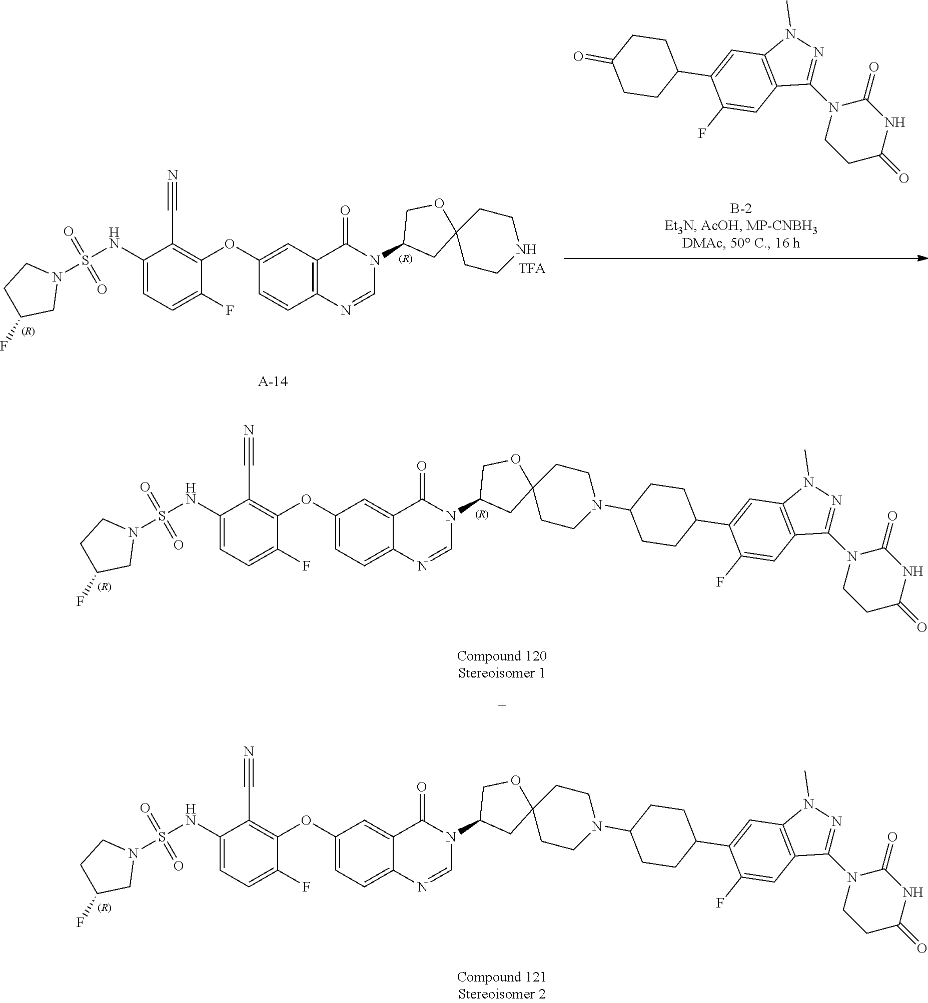

Into a 20 mL reaction vial containing a well stirred solution (3R)-N-[2-cyano-4-fluoro-3-[3-[(3R)-1-oxa-8-azaspiro[4.5]decan-3-yl]-4-oxo-quinazolin-6-yl]oxy-phenyl]-3-fluoro-pyrrolidine-1-sulfonamide (A-14) (500 mg, 713.64 mol, TFA salt) in DMAc (6 mL) was added NaOAc (159.81 mg, 1.95 mmol) at room temperature and the resulting mixture was stirred for 15 minutes at the same temperature. To the above mixture, 1-[5-fluoro-1-methyl-6-(4-oxocyclohexyl)indazol-3-yl]hexahydropyrimidine-2,4-dione (B-2) (306.89 mg, 856.37 mol) and AcOH (2.14 mmol, 122.43 μL) were added and stirring was continued at room temperature for 8 hours. Subsequently, MP-CNBH₃ (500 mg, 713.64 mol) was added and stirred at 50° C. for 16 hours. After the completion of the reaction, the reaction mixture was filtered through cotton pad and filtrate was concentrated under reduced pressure to get a crude residue which was purified by reverse phase column chromatography [Column: Redisep R_{f}Gold® reversed-phase C18-teledyne ISCO, 120 g), Mobile phase A: 10 mM NH₄HCO₃ in milli-Q water; Mobile phase B: acetonitrile]. Fractions were collected and lyophilized to afford a 124 mg mixture of cis and trans isomers. Further cis and trans isomers were separated by preparative HPLC [Column: X BRIDGE C18 (150 mm*21.0 mm, 5 μm), Mobile phase A: 10 mM NH₄HCO₃ in milli-Q water (pH-10 adjusted with ammonia), Mobile phase B: acetonitrile], flow: 15 mL/min). First eluting fraction (Peak-1) was lyophilized to afford Compound 120 (assigned as stereoisomer 1). Peak 1: (3R)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 120, 2.59 mg, 2.78 mol, 4% yield) as an off-white solid. UPLC-MS (ES+): m/z 929.6 [M+H]⁺. ¹H NMR (400 MHz, DMSO-d₆): δ 10.56 (s, 1H), 9.02 (bs, 1H), 8.32 (s, 1H), 7.76 (d, J=9.20 Hz, 1H), 7.64 (dd, J=2.80, 9.00 Hz, 1H), 7.54 (d, J=5.60 Hz, 1H), 7.40-7.30 (m, 4H), 5.35-5.18 (m, 2H), 4.28-4.10 (m, 2H), 4.00 (s, 3H), 3.91 (t, J=6.40 Hz, 2H), 3.27-3.10 (m, 5H), 3.00-2.80 (m, 2H), 2.75 (t, J=6.80 Hz, 4H), 2.30-1.35 (m, 17H). Similarly, second eluting fraction (Peak-2) was lyophilized to afford Compound 121 (assigned as stereoisomer 2).

Peak-2: (3R)-N-[2-cyano-3-[3-[(3R)-8-[4-[3-(2,4-dioxohexahydropyrimidin-1-yl)-5-fluoro-1-methyl-indazol-6-yl]cyclohexyl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 121, 15.18 mg, 16.33 mol, 2% yield) as an off-white solid. UPLC-MS (ES+): m/z 929.6 [M+H]⁺. ¹H NMR (400 MHz, DMSO-d₆): δ 10.56 (s, 1H), 8.58 (bs, 1H), 8.32 (s, 1H), 7.75 (d, J=8.80 Hz, 1H), 7.64 (dd, J=2.80, 9.00 Hz, 1H), 7.49-7.34 (m, 5H), 5.34-5.20 (m, 2H), 4.20-4.12 (m, 2H), 4.03 (s, 3H), 3.91 (t, J=6.80 Hz, 2H), 3.60-3.45 (m, 2H), 3.25-2.95 (m, 7H), 2.76 (t, J=6.80 Hz, 2H), 2.30-1.50 (m, 17H).

### Synthesis D63: Synthesis of (3R)-N-[2-Cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-fluoro-pyrrolidine-1-sulfonamide (Compound 122 and Compound 123)

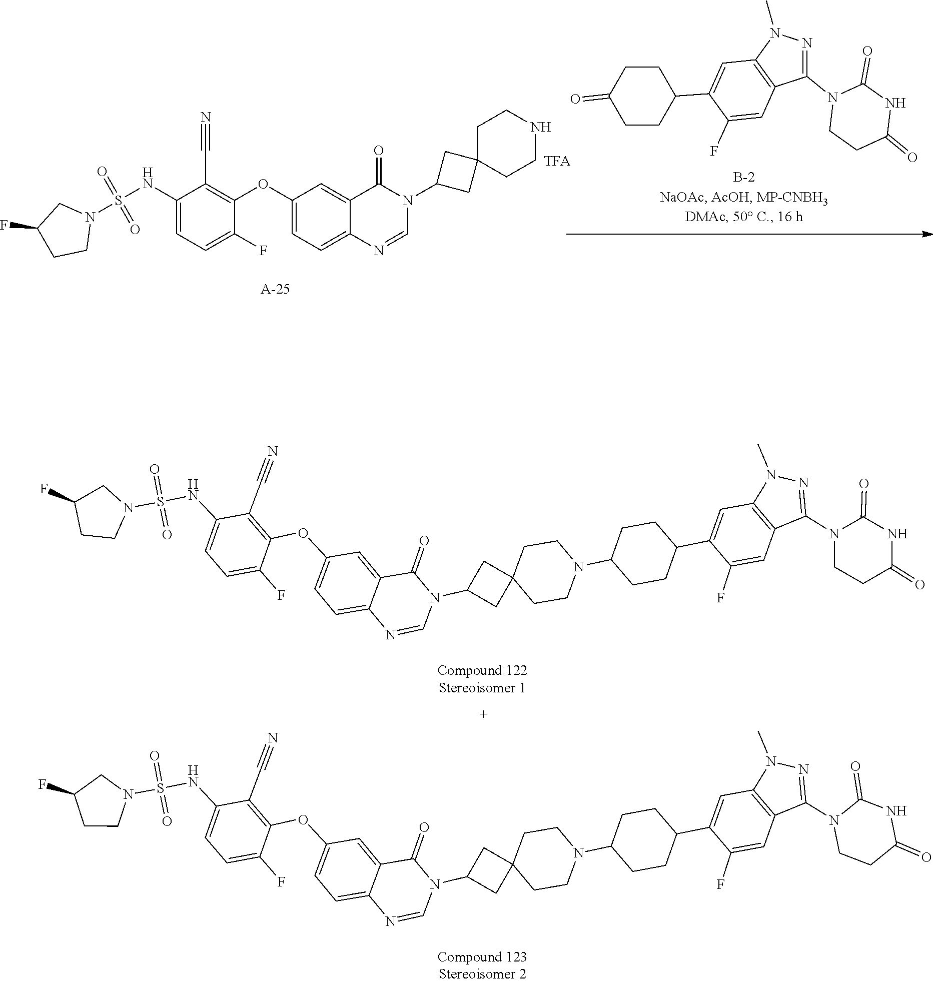

Into a 20 mL reaction vial containing a well stirred solution of of (3R)-N-[3-[3-(7-azaspiro[3.5]nonan-2-yl)-4-oxo-quinazolin-6-yl]oxy-2-cyano-4-fluoro-phenyl]-3-fluoro-pyrrolidine-1-sulfonamide (A-25) (500.00 mg, 730.32 mol, TFA salt) in DMAc (5.02 mL) was added NaOAc (119.82 mg, 1.46 mmol) at room temperature and the resulting mixture was stirred for 15 min at the same temperature. After 15 min, 1-[5-fluoro-1-methyl-6-(4-oxocyclohexyl)indazol-3-yl]hexahydropyrimidine-2,4-dione (B-2) (392.58 mg, 1.10 mmol) and AcOH (2.19 mmol, 125.3 μL) were added and stirring was continued at room temperature for 8 hours. Finally, MP-CNBH₃ (500 mg, 730.32 mol) was added and resulting reaction mixture was stirred at 50° C. for 16 hours. After the completion of the reaction, the reaction mixture was filtered through cotton pad and the filtrate was concentrated under reduced pressure to get a crude residue which was purified by reverse phase column chromatography [Column: Redisep R_{f} Gold® reversed-phase C18-teledyne ISCO, 120 g, Mobile phase A: 10 mM NH₄HCO₃ in milli-Q water; Mobile phase B: acetonitrile]. Fractions were collected and lyophilized to afford a 250 mg mixture of cis and trans isomers. Further cis and trans isomers were separated by preparative HPLC [Column: X BRIDGE C18 (150 mm*21.0 mm*5 μm), Mobile phase A: 10 mM NH₄HCO₃ in milli-Q water; (pH-10, adjusted with ammonia), Mobile phase B: acetonitrile], flow: 15 mL/min. First eluting fraction (Peak-1) was lyophilized to afford Compound 122 (assigned as stereoisomer 1).

Peak 1: (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-fluoro-pyrrolidine-1-sulfonamide (Compound 122, 5.3 mg, 5.65 mol, 8% yield) as an off-white solid. UPLC-MS (ES+): m/z 913.3 [M+H]⁺. ¹H NMR (400 MHz, DMSO-d₆): δ 10.56 (s, 1H), 8.89 (bs, 1H), 8.38 (s, 1H), 7.75 (d, J=8.80 Hz, 1H), 7.62 (dd, J=3.20, 9.00 Hz, 1H), 7.54 (d, J=5.60 Hz, 1H), 7.40-7.29 (m, 4H), 5.33-5.02 (m, 1H), 5.00-4.94 (m, 1H), 4.00 (s, 3H), 3.90 (t, J=6.80 Hz, 2H), 3.60-3.40 (m, 3H), 3.28-3.05 (m, 5H), 3.00-2.85 (m, 2H), 2.75 (t, J=6.80 Hz, 2H), 2.48-2.42 (m, 2H), 2.25-1.50 (m, 15H).

Similarly, the second eluting fraction (Peak-2) was lyophilized to afford Compound 123 (assigned as stereoisomer 2).

Peak-2: (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]-4-oxo-quinazolin-6-yl]oxy-4-fluoro-phenyl]-3-fluoro-pyrrolidine-1-sulfonamide (Compound 123, 21.4 mg, 23.39 mol, 3% yield) as an off-white solid. UPLC-MS (ES+): m/z 913.6 [M+H]⁺. ¹H NMR (400 MHz, DMSO-d₆): δ 10.56 (s, 1H), 8.53 (bs, 1H), 8.38 (s, 1H), 7.75 (d, J=8.80 Hz, 1H), 7.62 (dd, J=2.80, 9.00 Hz, 1H), 7.49 (d, J=5.60 Hz, 1H), 7.41-7.31 (m, 4H), 5.34-5.20 (m, 1H), 4.98-4.93 (m, 1H), 4.03 (s, 3H), 3.91 (t, J=6.40 Hz, 2H), 3.68-3.40 (m, 4H), 3.28-2.90 (m, 7H), 2.76 (t, J=6.80 Hz, 2H), 2.40-2.30 (m, 3H), 2.20-1.35 (m, 14H).

### Synthesis D64: Synthesis of 7-[2-Cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]oxy]quinoxaline (Compound 124 and Compound 125)

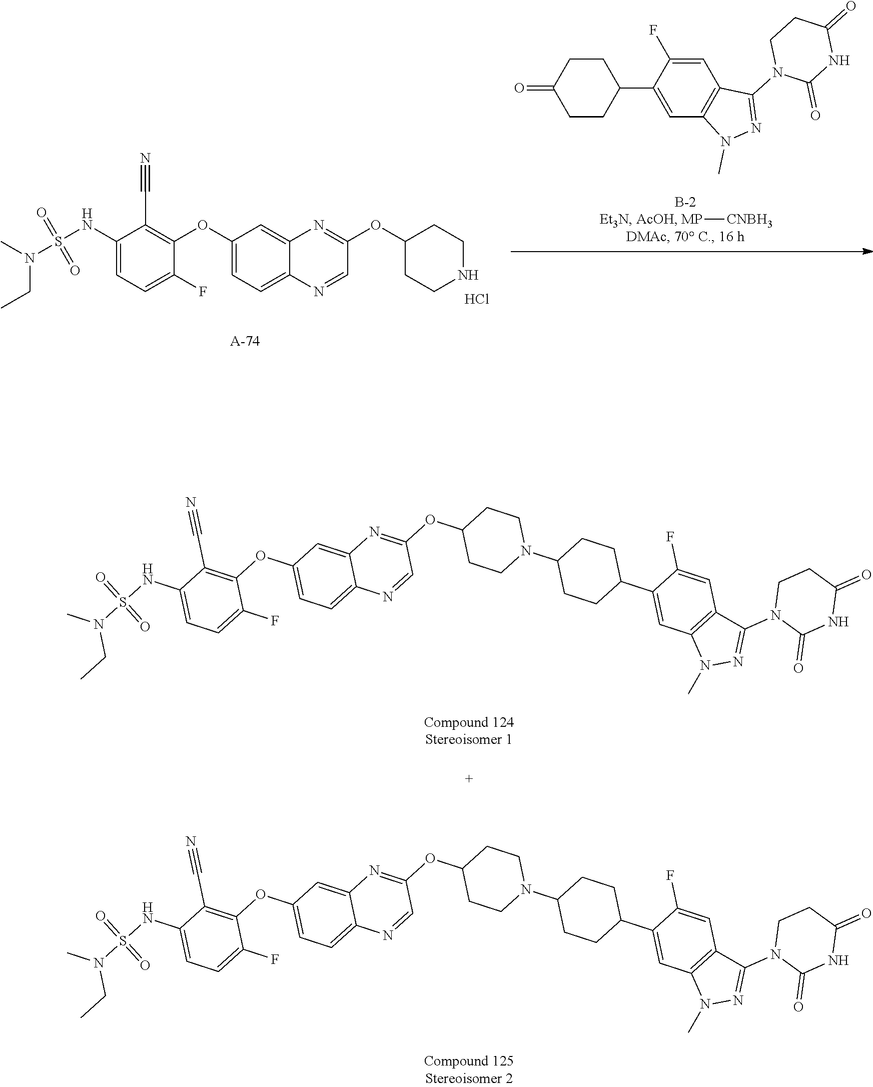

Into a 20 mL pressure vial containing the well stirred solution of 7-[2-cyano-3-[[ethyl(methyl)sulfamoyl]amino]-6-fluoro-phenoxy]-2-(4-piperidyloxy)quinoxaline (A-74) (600 mg, 804.46 mol, HCl salt) in DMAc (10.04 mL) was added triethylamine (4.02 mmol, 560.63 L) at room temperature and stirred for 30 minutes at the same temperature. To the above mixture, 1-[5-fluoro-1-methyl-6-(4-oxocyclohexyl)indazol-3-yl]hexahydropyrimidine-2,4-dione (B-2) (288.29 mg, 804.46 mol) and AcOH (2.41 mmol, 138.01 μL) were added at 0° C. and resulting reaction mixture was stirred at room temperature for 3 hours. Subsequently, MP-CNBH₃ (600 mg) was added and stirred at 70° C. for 16 hours. After completion of the reaction, the reaction mixture was filtered through a cotton pad and cotton pad was washed with dichloromethane. Filtrate was concentrated under reduced pressure to get a crude residue which was purified by preparative HPLC [Column: X-SELECT CSH (150 mm*19 mm*5 μm), Mobile phase A: 10 mm NH₄HCO₃ in acetonitrile; Mobile phase B: 100% acetonitrile]. The fast-eluting fraction (Peak-1) was lyophilized to afford Compound 124 (assigned as stereoisomer 1).

Peak-1: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]oxy]quinoxaline (Compound 124, 32.06 mg, 37.44 mol, 5% yield) as an off-white solid. UPLC-MS (ES+): m/z 843.4 [M+H]⁺. ¹H NMR (400 MHz, DMSO-d₆): δ 10.54 (s, 1H), 9.20 (bs, 1H), 8.50 (s, 1H), 8.07 (d, J=9.20 Hz, 1H), 7.54-7.30 (m, 5H), 6.90 (s, 1H), 5.60-5.30 (m, 1H), 4.00 (s, 3H), 3.91 (t, J=6.80 Hz, 2H), 3.22-2.82 (m, 5H), 2.76 (t, J=6.40 Hz, 2H), 2.61 (s, 3H), 2.45-2.35 (m, 2H), 2.30-1.50 (m, 13H), 1.04 (t, J=7.20 Hz, 3H).

Similarly, second eluted fraction (Peak-2) was lyophilized to afford Compound 125 (assigned as stereoisomer 2).

Peak-2: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]oxy]quinoxaline (Compound 125, 41.46 mg, 47.80 mol, 6% yield) as an off white solid. UPLC-MS (ES+): m/z 843.4 [M+H]⁺. ¹H NMR (400 MHz, DMSO-d₆): δ 10.54 (s, 1H), 8.79 (bs, 1H), 8.49 (s, 1H), 8.06 (d, J=9.20 Hz, 1H), 7.52-7.37 (m, 5H), 6.93 (s, 1H), 5.60-5.20 (m, 1H), 4.03 (s, 3H), 3.92 (t, J=6.80 Hz, 2H), 3.69-3.42 (m, 1H), 3.21-2.81 (m, 5H), 2.76 (t, J=6.40 Hz, 2H), 2.62-2.54 (m, 4H), 2.29-1.50 (m, 13H), 1.04 (t, J=7.20 Hz, 3H).

### Synthesis D65: Synthesis of 6-[2-cyano-3-[[ethyl(methyl)sulfamoyl]amino]-6-fluoro-phenoxy]-3-[[1-[4-[3-(2,4-dioxohexahydropyrimidin-1-yl)-5-fluoro-1-methyl-indazol-6-yl]cyclohexyl]-4-piperidyl]methyl]-4-oxo-quinazoline (Compound 126 and Compound 127)

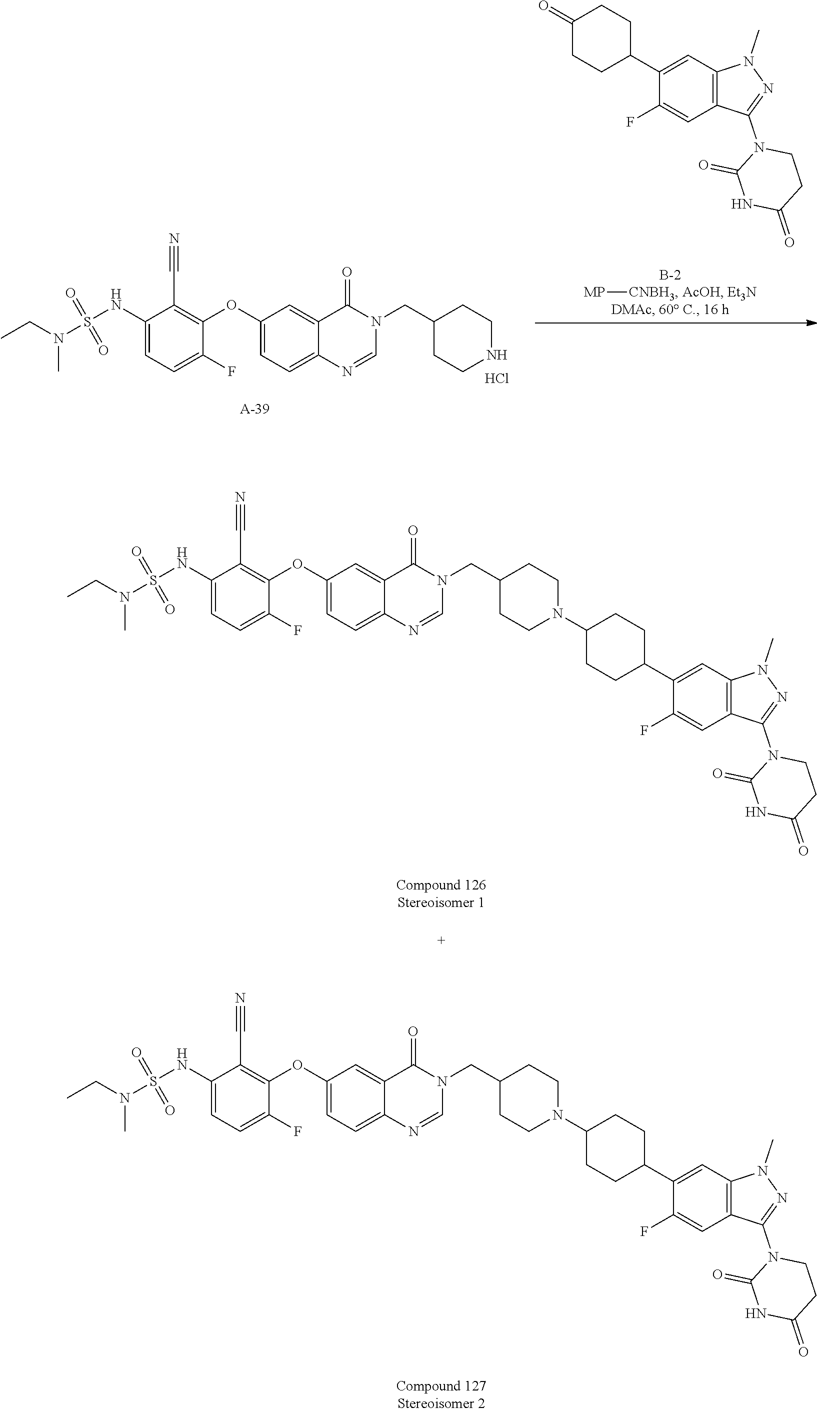

Into 20 mL pressure vial containing a well stirred solution 6-[2-cyano-3-[[ethyl(methyl)sulfamoyl]amino]-6-fluoro-phenoxy]-4-oxo-3-(4-piperidylmethyl)quinazoline (A-39) (500 mg, 907.39 mol, HCl salt) in DMAc (3 mL) was added triethylamine (2.72 mmol, 379.42 L) at 0° C. and stirred for 30 minutes at room temperature. After 30 min, 1-[5-fluoro-1-methyl-6-(4-oxocyclohexyl)indazol-3-yl]hexahydropyrimidine-2,4-dione (B-2) (390.21 mg, 1.09 mmol) and AcOH (4.54 mmol, 254.62 μL) were added at 0° C. and stirred for 2 hours at room temperature. Subsequently, MP-CNBH₃ (500 mg, 907.39 mol) was added at room temperature. and resulting reaction mixture was stirred at 60° C. for 16 hours. The reaction mixture was filtered through cotton pad and filtrate was concentrated under reduced pressure to get a crude residue which was purified by reverse phase column chromatography preparative HPLC [Column: YMC ACTUS C18 (250 mm*20 mm*5 m), Mobile phase A: 10 mm NH₄HCO₃ in milli-Q water; Mobile phase B: 100% acetonitrile, flow rate: 15 mL/min]. The first eluting fraction was collected and lyophilized to afford Compound 126 (assigned as stereoisomer 1).

Peak-1 6-[2-cyano-3-[[ethyl(methyl)sulfamoyl]amino]-6-fluoro-phenoxy]-3-[[1-[4-[3-(2,4-dioxohexahydropyrimidin-1-yl)-5-fluoro-1-methyl-indazol-6-yl]cyclohexyl]-4-piperidyl]methyl]-4-oxo-quinazoline (Compound 126, 51.9 mg, 60.04 mol, 7% yield) as an off-white solid. UPLC-MS (ES+): m/z 856.9 [M+H]⁺. ¹H NMR (400 MHz, DMSO-d₆): δ 10.55 (s, 1H), 8.81 (bs, 1H), 8.35 (s, 1H), 7.77 (d, J=9.20 Hz, 1H), 7.65 (dd, J=3.20, 8.80 Hz, 1H), 7.52 (d, J=5.60 Hz, 1H), 7.38-7.25 (m, 4H), 3.98-3.88 (m, 7H), 3.50-3.38 (m, 2H), 3.18-2.80 (m, 7H), 2.75 (t, J=6.80 Hz, 2H), 2.62-2.58 (m, 3H), 2.18-1.95 (m, 5H), 1.80-1.40 (m, 9H), 1.03 (t, J=7.20 Hz, 3H).

Similarly, the second eluted fraction was collected and lyophilized to afford Compound 127 (assigned as stereoisomer 2).

Peak-2: 6-[2-cyano-3-[[ethyl(methyl)sulfamoyl]amino]-6-fluoro-phenoxy]-3-[[1-[4-[3-(2,4-dioxohexahydropyrimidin-1-yl)-5-fluoro-1-methyl-indazol-6-yl]cyclohexyl]-4-piperidyl]methyl]-4-oxo-quinazoline (Compound 127, 41.17 mg, 47.81 mol, 5% yield) as an off-white solid. UPLC-MS (ES+): m/z 857.5 [M+H]⁺. ¹H NMR (400 MHz, DMSO-d₆): δ 10.56 (s, 1H), 8.80-8.35 (m, 2H), 7.77 (d, J=9.20 Hz, 1H), 7.67-7.29 (m, 6H), 4.02 (s, 3H), 3.99-3.89 (m, 4H), 3.70-3.50 (m, 1H), 3.21-2.83 (m, 5H), 2.76 (t, J=6.80 Hz, 2H), 2.65-2.55 (m, 4H), 2.15-1.40 (m, 14H), 1.03 (t, J=6.80 Hz, 3H).

### Synthesis D66: Synthesis of 7-[2-cyano-3-[[ethyl(methyl)sulfamoyl]amino]-6-fluoro-phenoxy]-2-[2-[1-[4-[3-(2,4-dioxohexahydropyrimidin-1-yl)-5-fluoro-1-methyl-indazol-6-yl]cyclohexyl]-4-piperidyl]ethyl]quinoxaline (Compound 128 and Compound 129)

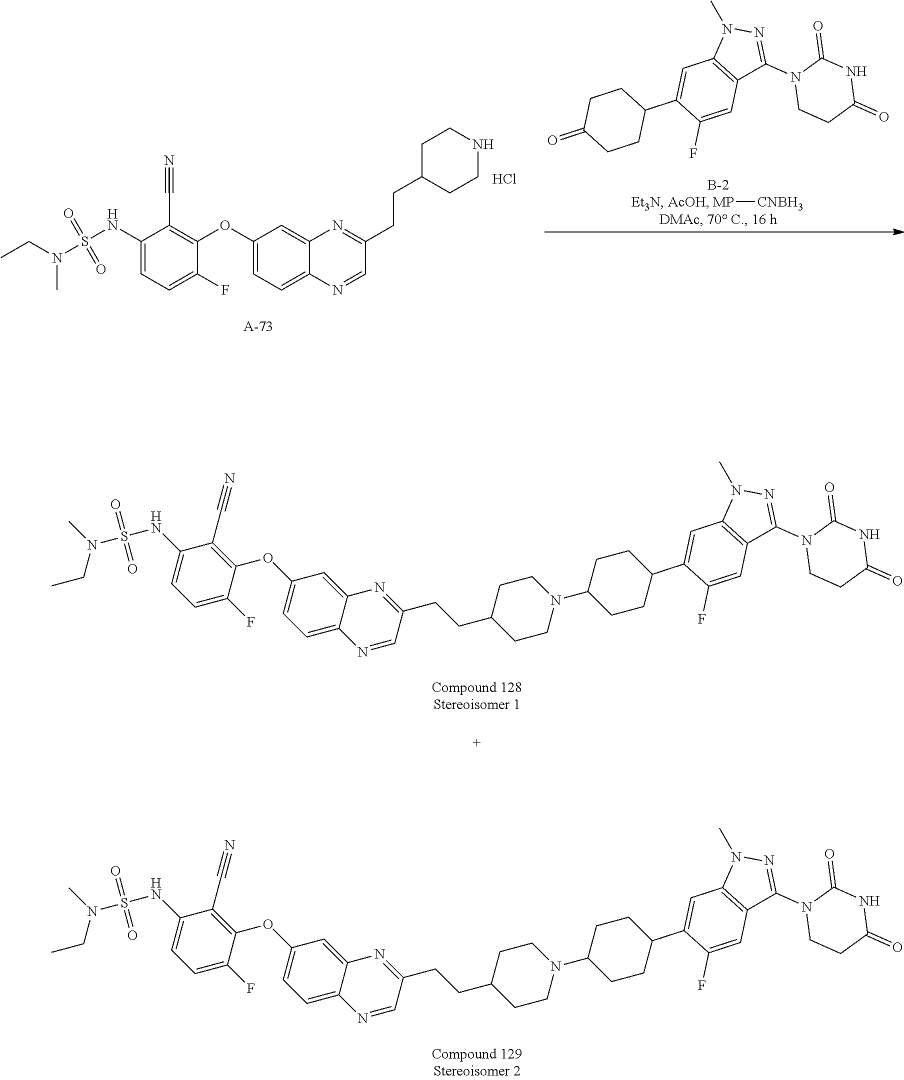

Into a 20 mL pressure vial containing a well-stirred solution of 7-[2-cyano-3-[[ethyl(methyl)sulfamoyl]amino]-6-fluoro-phenoxy]-2-[2-(4-piperidyl)ethyl]quinoxaline (A-73) (350 mg, 484.72 mol, HCl salt) in DMAc (10.14 mL) was added triethylamine (2.42 mmol, 337.80 μL) at room temperature under nitrogen atmosphere and mixture was stirred for 30 min. Reaction mixture was cooled to 0° C. and was added 1-[5-fluoro-1-methyl-6-(4-oxocyclohexyl)indazol-3-yl]hexahydropyrimidine-2,4-dione (B-2) (173.71 mg, 484.72 μmol) followed by AcOH (1.45 mmol, 83.16 μL) and resulting mixture was stirred for 2 hours at room temperature. Subsequently, MP-CNBH₃ (350 mg, 484.72 μmol) was added, and reaction mixture was stirred at 70° C. for 16 hours. After completion of the reaction, the reaction mixture was filtered and filtrate was concentrated under reduced pressure to get a crude residue which was purified by preparative HPLC [Column: X-Bridge C18 (150 mm*21 mm*5 m), Mobile phase A: 10 mM NH₄HCO₃ in milli-Q water (pH was adjusted rto 10 with ammonia); Mobile phase B: acetonitrile]. First eluting fraction (Peak-1) was concentrated under reduced pressure at 40° C. and lyophilized to afford Compound 128 (assigned as stereoisomer 1).

Peak-1: 7-[2-cyano-3-[[ethyl(methyl)sulfamoyl]amino]-6-fluoro-phenoxy]-2-[2-[1-[4-[3-(2,4-dioxohexahydropyrimidin-1-yl)-5-fluoro-1-methyl-indazol-6-yl]cyclohexyl]-4-piperidyl]ethyl]quinoxaline (Compound 128, 20 mg, 23.27 mol, 5% yield) as an off-white solid. UPLC-MS (ES+): m/z 855.5 [M+H]⁺. ¹H NMR (400 MHz, DMSO-d₆): δ 10.54 (s, 1H), 8.90-8.75 (m, 2H), 8.12 (d, J=9.20 Hz, 1H), 7.66 (dd, J=2.40, 9.20 Hz, 1H), 7.53 (d, J=5.60 Hz, 1H), 7.47-7.21 (m, 3H), 7.10 (bs, 1H), 3.99 (s, 3H), 3.90 (t, J=6.80 Hz, 2H), 3.61-3.41 (m, 1H), 3.09-2.89 (m, 7H), 2.75 (t, J=6.80 Hz, 2H), 2.63-2.56 (m, 4H), 2.22-2.11 (m, 2H), 2.09-1.91 (m, 4H), 1.88-1.50 (m, 8H), 1.49-1.37 (m, 2H), 1.03 (t, J=7.20 Hz, 3H).

Similarly, the second eluted fraction (Peak-2) was concentrated under reduced pressure at 40° C. and lyophilized to afford Compound 129 (assigned as stereoisomer 2).

Peak-2: 7-[2-cyano-3-[[ethyl(methyl)sulfamoyl]amino]-6-fluoro-phenoxy]-2-[2-[1-[4-[3-(2,4-dioxohexahydropyrimidin-1-yl)-5-fluoro-1-methyl-indazol-6-yl]cyclohexyl]-4-piperidyl]ethyl]quinoxaline (Compound 129, 24 mg, 27.27 mol, 6% yield) as an off-white solid. UPLC-MS (ES+): m/z 855.5 [M+H]⁺. ¹H NMR (400 MHz, DMSO-d₆): δ 10.55 (s, 1H), 8.84 (s, 1H), 8.48 (bs, 1H), 8.12 (d, J=9.20 Hz, 1H), 7.66 (dd, J=2.80, 9.20 Hz, 1H), 7.48-7.32 (m, 4H), 7.11 (bs, 1H), 4.03 (s, 3H), 3.91 (t, J=6.80 Hz, 2H), 3.62-3.51 (m, 1H), 3.22-3.12 (m, 2H), 3.09-2.82 (m, 6H), 2.79-2.71 (m, 2H), 2.63-2.57 (m, 3H), 2.18-1.33 (m, 16H), 1.03 (t, J=7.20 Hz, 3H).

### Synthesis D67: Synthesis of (3R)-3-[6-[2-cyano-6-fluoro-3-[[2-hydroxyethyl(methyl)sulfamoyl]amino]phenoxy]-4-oxo-quinazolin-3-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 130)

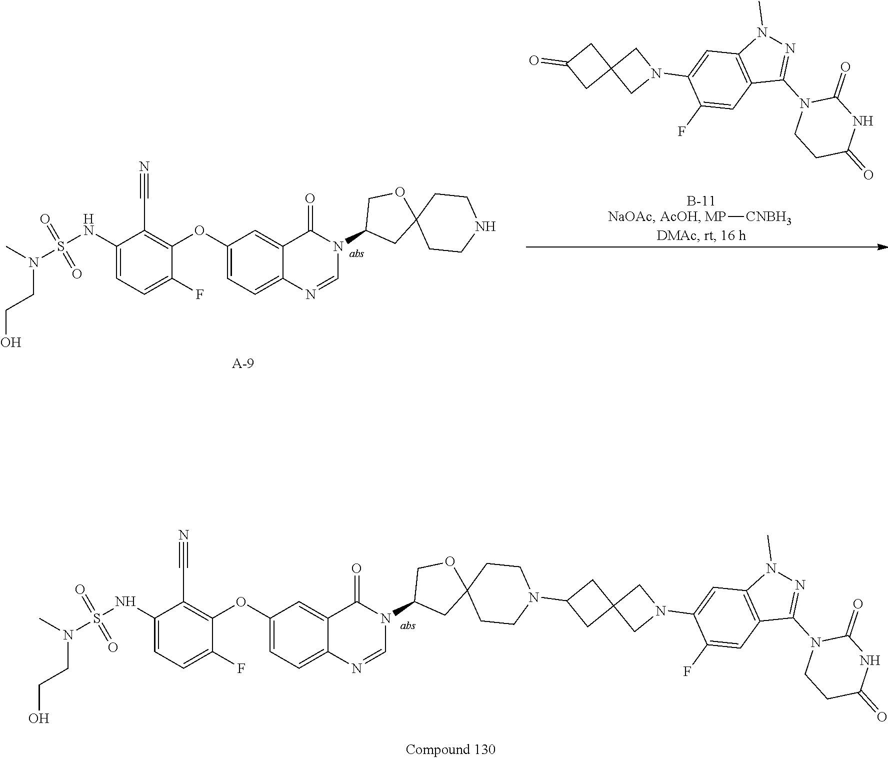

Into a 20 mL pressure vial containing a well-stirred solution of (3R)-3-[6-[2-cyano-6-fluoro-3-[[2-hydroxyethyl(methyl)sulfamoyl]amino]phenoxy]-4-oxo-quinazolin-3-yl]-1-oxa-8-azaspiro[4.5]decane (A-9) (100 mg, 148.44 μmol) in DMAc (3 mL) was added anhydrous sodium acetate (60.88 mg, 742.22 mol) at room temperature under nitrogen atmosphere and mixture was stirred for 10 minutes. Reaction mixture was cooled to 0° C. and was added 1-[5-fluoro-1-methyl-6-(6-oxo-2-azaspiro[3.3]heptan-2-yl)indazol-3-yl]hexahydropyrimidine-2,4-dione (B-11) (74.33 mg, 178.13 μmol) followed by AcOH (445.33 μmol, 25.46 μL) and resulting mixture was stirred for 30 minutes at room temperature. Finally, MP-CNBH₃ (100 mg, 148.44 μmol) was added, and the reaction mixture was stirred at room temperature for 16 hours. After completion of the reaction, the reaction mixture was filtered and filtrate was concentrated under reduced pressure to get a crude residue which was purified by reverse phase using the method (Redisep R_{f}Gold®reversed-phase C18-teledyne ISCO, 100 g), [Mobile phase A: 10 mM NH₄HCO₃ in mill-Q water; Mobile phase B: acetonitrile] to afford (3R)-3-[6-[2-cyano-6-fluoro-3-[[2-hydroxyethyl(methyl)sulfamoyl]amino]phenoxy]-4-oxo-quinazolin-3-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 130, 24 mg, 25.51 μmol, 17% yield) as an off-white solid. UPLC-MS (ES+): m/z 928.6 [M+H]⁺. ¹H NMR (400 MHz, DMSO-d₆): δ 10.48 (s, 1H), 8.31 (s, 1H), 7.77 (d, J=8.80 Hz, 1H), 7.65-7.49 (m, 2H), 7.37-7.25 (m, 3H), 6.51 (d, J=7.20 Hz, 1H), 5.28 (quint, J=6.8 Hz, 1H), 4.20-4.09 (m, 2H), 4.02 (s, 3H), 3.93-3.84 (m, 7H), 3.52-3.49 (m, 2H), 3.08 (t, J=6.00 Hz, 2H), 2.88-2.70 (m, 8H), 2.49-2.43 (m, 1H), 2.42-2.33 (m, 1H), 2.32-2.22 (m, 2H), 2.18-2.08 (m, 1H), 2.00-1.68 (m, 4H).

### Synthesis D68: Synthesis of 7-[2-cyano-3-[[ethyl(methyl)sulfamoyl]amino]-6-fluoro-phenoxy]-2-[2-[1-[4-[4-[(2,6-dioxo-3-piperidyl)amino]-2-fluoro-phenyl]cyclohexyl]-4-piperidyl]ethyl]quinoxaline (Compound 131 and Compound 132)

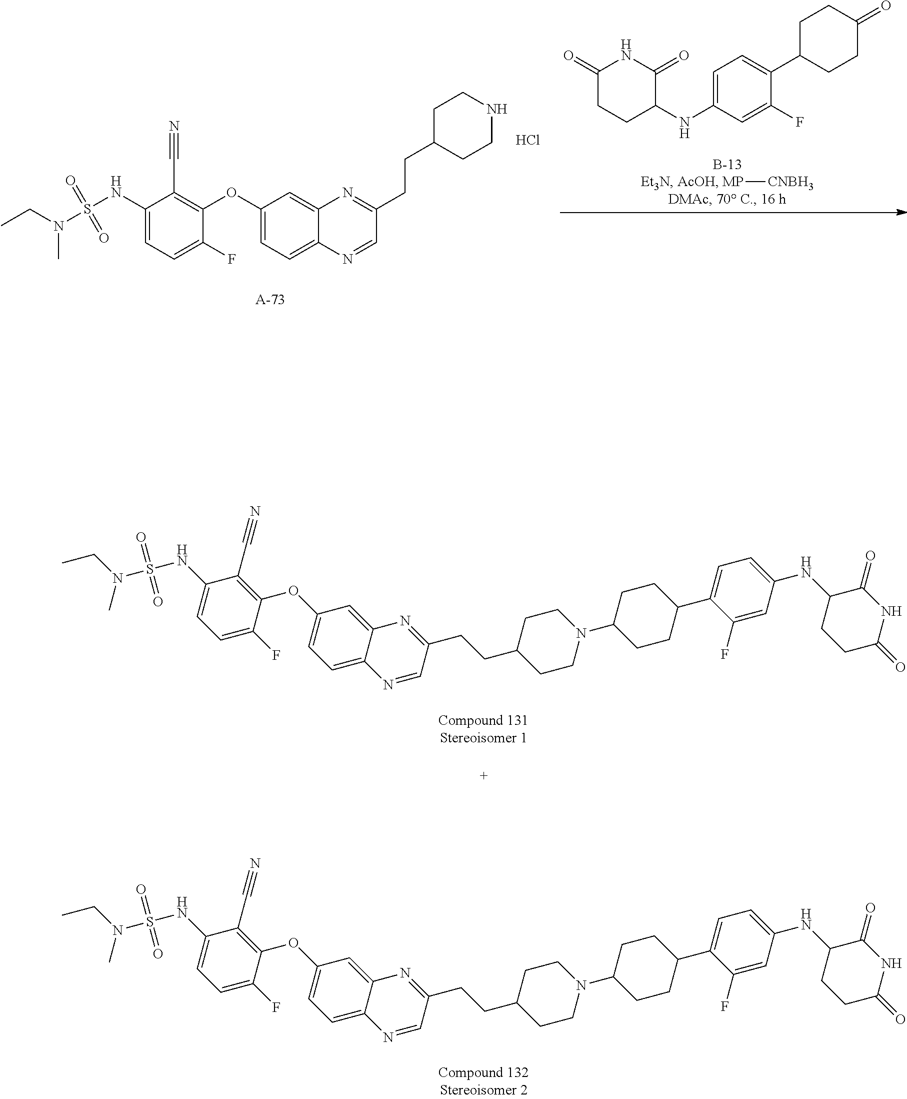

Into a 20 mL pressure vial containing a well-stirred solution of 7-[2-cyano-3-[[ethyl(methyl)sulfamoyl]amino]-6-fluoro-phenoxy]-2-[2-(4-piperidyl)ethyl]quinoxaline (A-73) (350 mg, HCl Salt) in DMAc (6.15 mL) was added triethylamineutes (2.45 mmol, 342.07 μL) at room temperature under nitrogen atmosphere and mixture was stirred for 30 min. To the above mixture, 3-[3-fluoro-4-(4-oxocyclohexyl)anilino]piperidine-2,6-dione (B-13) (156.25 mg, 490.84 mol) and AcOH (1.47 mmol, 84.20 μL) were added and stirred for 5 hours at room temperature. Subsequently, MP-CNBH₃ (800 mg, 490.84 mol) was added, and the resulting reaction mixture was stirred at 70° C. for 16 hours. After completion of the reaction, the reaction mixture was filtered and filtrate was concentrated under reduced pressure to get a crude residue which was purified by reverse phase column chromatography (Redisep R_{f}Gold® C18-teledyne ISCO, 120 g), [Mobile phase A: 10 mM NH₄HCO₃ in milli-Q water; Mobile phase B: acetonitrile]. Fractions were lyophilized to afford mixture of cis and trans isomers. Further cis and trans isomers were separated by preparative HPLC [Column: X-Bridge C18 (150 mm*19 mm*5 m), Mobile phase A: 10 mM NH₄HCO₃ in milli-Q water; Mobile phase B: acetonitrile]. First eluting fraction (Peak-1) was lyophilized to afford Compound 131 (assigned as stereoisomer 1).

Peak-1: 7-[2-cyano-3-[[ethyl(methyl)sulfamoyl]amino]-6-fluoro-phenoxy]-2-[2-[1-[4-[4-[(2,6-dioxo-3-piperidyl)amino]-2-fluoro-phenyl]cyclohexyl]-4-piperidyl]ethyl]quinoxaline (Compound 131.32 mg, 38.76 mol, 8% yield) as an off-white solid. UPLC-MS (ES+): m/z 815.7 [M+H]⁺. ¹H NMR (400 MHz, DMSO-d₆): δ 10.72 (m, 1H), 8.79 (s, 1H), 8.10 (d, J=9.20 Hz, 1H), 7.62 (dd, J=2.80, 9.20 Hz, 1H), 7.39-7.30 (m, 1H), 7.29-7.20 (m, 1H), 7.09 (d, J=2.4 Hz, 1H), 7.02-6.93 (m, 1H), 6.51-6.32 (m, 2H), 5.96 (d, J=7.60 Hz, 1H), 4.32-4.26 (m, 1H), 2.99-2.91 (m, 6H), 2.80-2.71 (m, 1H), 2.61-2.59 (m, 1H), 2.56-2.53 (m, 5H), 2.33-2.24 (m, 2H), 2.15-2.00 (m, 1H), 1.93-1.54 (m, 9H), 1.53-1.33 (m, 4H), 1.32-1.11 (m, 3H), 1.04 (t, J=Hz, 3H).

Similarly, the second eluted fraction (Peak-2) was lyophilized to afford Compound 132 (assigned as stereoisomer 2).

Peak-2: 7-[2-cyano-3-[[ethyl(methyl)sulfamoyl]amino]-6-fluoro-phenoxy]-2-[2-[1-[4-[4-[(2,6-dioxo-3-piperidyl)amino]-2-fluoro-phenyl]cyclohexyl]-4-piperidyl]ethyl]quinoxaline (Compound 132.23 mg, 27.81 mol, 6% yield) as an off-white solid. UPLC-MS (ES+): m/z 815.5 [M+H]⁺. ¹H NMR (400 MHz, DMSO-d₆): δ 10.77 (s, 1H), 8.81 (s, 1H), 8.11 (d, J=9.20 Hz, 1H), 7.62 (dd, J=2.80, 8.80 Hz, 1H), 7.43-7.32 (m, 1H), 7.31-7.22 (m, 1H), 7.19-7.00 (m, 2H), 6.54-6.38 (m, 2H), 5.99 (d, J=7.60 Hz, 1H), 4.33-4.27 (m, 1H), 3.05-2.85 (m, 7H), 2.84-2.63 (m, 3H), 2.57-2.55 (m, 3H), 2.13-1.99 (m, 1H), 1.99-1.70 (m, 11H), 1.70-1.56 (m, 2H), 1.55-1.30 (m, 3H), 1.02 (t, J=7.20 Hz, 3H).

### Synthesis D69: Synthesis of (3R)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 133)

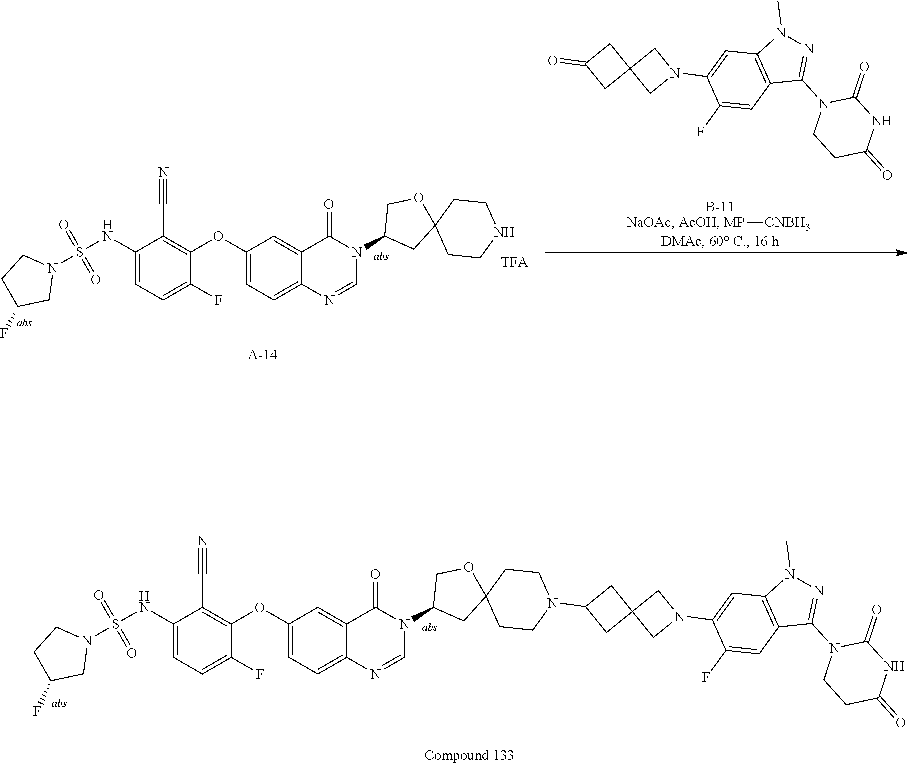

Into a 20 mL glass vial containing a well-stirred solution of (3R)-N-[2-cyano-4-fluoro-3-[3-[(3R)-1-oxa-8-azaspiro[4.5]decan-3-yl]-4-oxo-quinazolin-6-yl]oxy-phenyl]-3-fluoro-pyrrolidine-1-sulfonamide (A-14) (150.00 mg, 214.09 mol, TFA salt) and 1-[5-fluoro-1-methyl-6-(6-oxo-2-azaspiro[3.3]heptan-2-yl)indazol-3-yl]hexahydropyrimidine-2,4-dione (B-11) (79.51 mg, 214.09 mol) in DMAc (5 mL) was added NaOAc (87.81 mg, 1.07 mmol) and the resulting mixture was stirred at room temperature for 1 hour. Subsequently, AcOH (642.28 mol, 36.73 μL) added and stirred at room temperature for 2 hours. Finally, MP-CNBH₃ (0.225 g, 214.09 mol) was added and stirred at 60° C. for 16 hours. After completion the reaction, reaction mixture was filtered through sintered funnel and the filtrate was concentrated under reduced pressure to get crude, which was purified by the reverse phase purification [Column: Redisep R_{f} Gold® C18-teledyne ISCO, 100 g), [Mobile phase A: 10 mM NH₄HCO₃ in milli-Q water; Mobile phase B: acetonitrile]. Pure fractions were collected and lyophilized to afford (3R)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 133, 0.047 g, 48.40 mol, 23% yield) as a white solid. UPLC-MS (ES+): m/z 942.6 [M+H]⁺. ¹H NMR (400 MHz, DMSO-d₆): δ 10.51 (s, 1H), 9.51 (bs, 1H), 8.32 (s, 1H), 7.76 (d, J=9.20 Hz, 1H), 7.65 (dd, J=3.20, 9.3 Hz, 1H), 7.47 (bs, 1H), 7.35-7.32 (m, 2H), 7.27 (d, J=12.40 Hz, 1H), 6.52 (d, J=7.60 Hz, 1H), 5.36-5.22 (m, 2H), 4.21-4.11 (m, 2H), 4.03 (s, 2H), 3.93 (s, 2H), 3.88-3.86 (m, 5H), 3.68-3.37 (m, 2H), 3.29-3.23 (m, 4H), 2.92-2.67 (m, 5H), 2.56-2.50 (m, 2H), 2.42-2.33 (m, 2H), 2.20-1.72 (m, 8H).

### Synthesis D70: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]propane-2-sulfonamide (Compound 134)

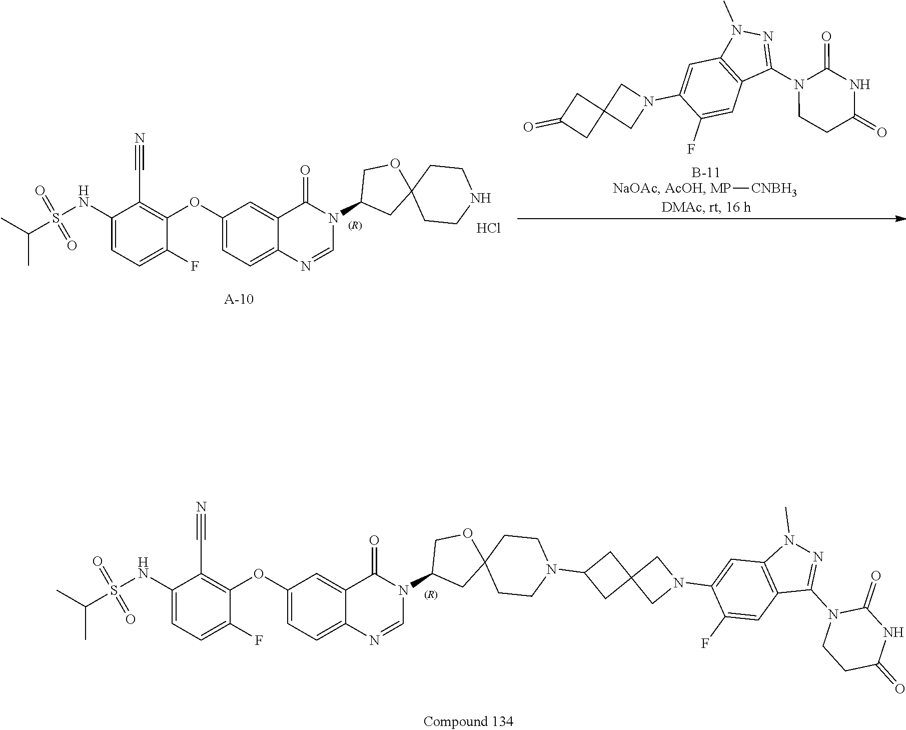

Into 20 mL glass vial containing a well stirred solution N-[2-cyano-4-fluoro-3-[3-[(3R)-1-oxa-8-azaspiro[4.5]decan-3-yl]-4-oxo-quinazolin-6-yl]oxy-phenyl]propane-2-sulfonamide (A-10) (200.00 mg, 322.81 mol, HCl salt) and 1-[5-fluoro-1-methyl-6-(6-oxo-2-azaspiro[3.3]heptan-2-yl)indazol-3-yl]hexahydropyrimidine-2,4-dione (B-11) (193.35 mg, 322.81 mol) in anhydrous DMAc (4 mL) was added anhydrous NaOAc (52.96 mg, 645.61 mol) followed by AcOH (645.61 mol, 36.92 μL) at room temperature and mixture was stirred for 10 min. Subsequently, MP-CNBH₃ (200 mg, 322.81 mol) was added and resulting reaction mixture was stirred at room temperature for 16 hours. The reaction mass was diluted with dichloromethane (10 mL) and filtered through cotton. Filtrate was concentrated under reduced pressure to afford a crude residue which was purified by preparative HPLC (Column: X-BRIDGE C8 (250 mm*19 mm*5 μm), Mobile phase A: 10 mM NH₄HCO₃ in milli-Q water; Mobile phase B: acetonitrile]. Fraction was lyophilized to get N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]propane-2-sulfonamide (Compound 134, 28 mg, 31.04 mol, 9.62% yield, 99.44% purity) as an off-white solid. UPLC-MS (ES+): m/z 897.2 [M+H]⁺. ¹H NMR (400 MHz, DMSO-d₆): δ 10.50 (s, 1H), 9.76 (bs, 1H), 8.32 (s, 1H), 7.76 (d, J=8.80 Hz, 1H), 7.65 (dd, J=3.20, 8.80 Hz, 1H), 7.49 (bs, 1H), 7.42-7.33 (m, 2H), 7.27 (d, J=12.80 Hz, 1H), 6.52 (d, J=7.20 Hz, 1H), 5.29 (bs, 1H), 4.18-4.10 (m, 2H), 4.02 (s, 2H), 3.92-3.86 (m, 7H), 3.25-2.80 (m, 4H), 2.73 (t, J=6.80 Hz, 3H), 2.41-2.38 (m, 1H), 2.36-2.07 (m, 4H), 2.00-1.55 (m, 5H), 1.25 (d, J=6.40 Hz, 7H).

### Synthesis D71: Synthesis of N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]cyclopentanesulfonamide (Compound 135 and Compound 136)

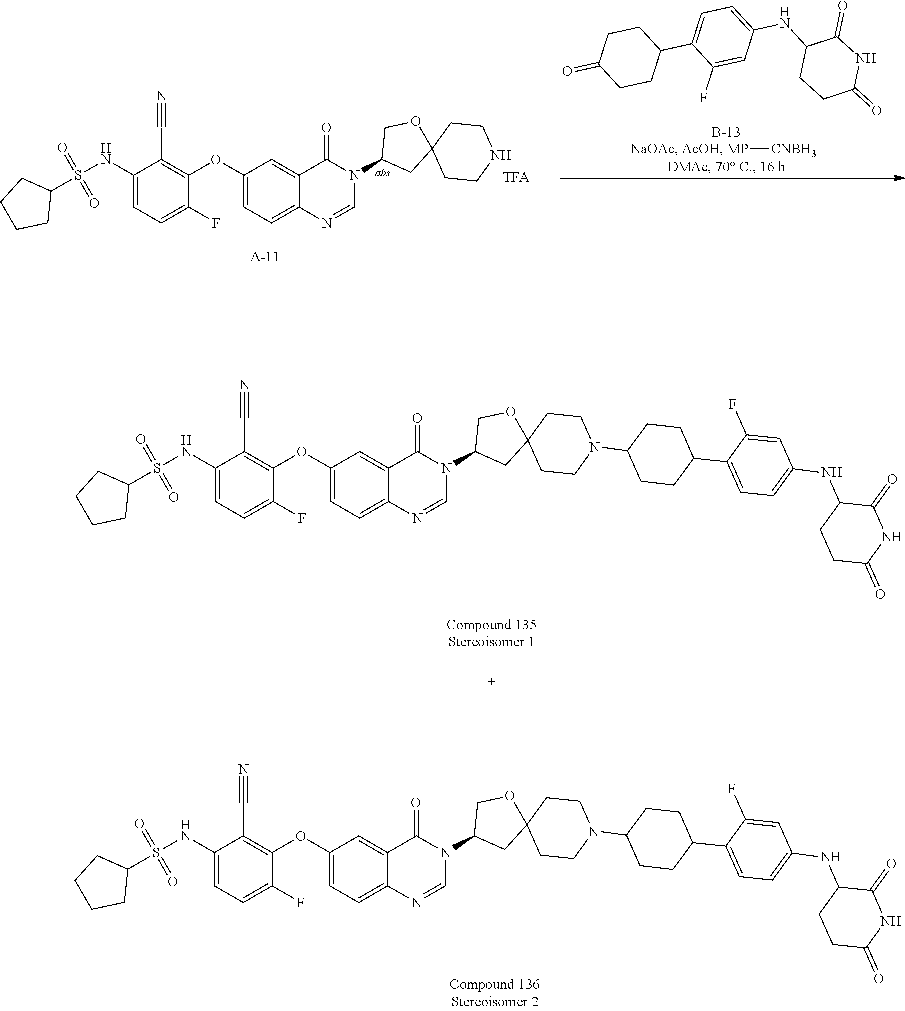

Into a 20 mL pressure vial containing a well-stirred solution of N-[2-cyano-4-fluoro-3-3-[(3R)-1-oxa-8-azaspiro[4.5]decan-3-yl]-4-oxo-quinazolin-6-yl]oxy-phenyl]cyclopentanesulfonamide (A-11) (200 mg, 293.40 mol, TFA salt) in DMAc (5 mL) was added anhydrous NaOAc (24.07 mg, 293.40 mol, 15.75 μL) at room temperature under nitrogen atmosphere and mixture was stirred for 20 minutes. Reaction mixture was cooled to 0° C. and was added 3-[3-fluoro-4-(4-oxocyclohexyl)anilino]piperidine-2,6-dione (B-13) (93.40 mg, 293.40 mol) followed by AcOH (17.62 mg, 293.40 mol) and resulting mixture was stirred for 3 hours at room temperature. Finally, MP-CNBH₃ (200 mg, 293.40 mol) was added, and the reaction mixture was stirred at 70° C. for 16 hours. After completion of the reaction, the reaction mixture was filtered and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water; Mobile phase B: acetonitrile]. The first eluting fraction (peak-1) was concentrated under reduced pressure at 40° C. and lyophilized to afford Compound 135 (assigned as stereoisomer 1).

Peak-1: N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]cyclopentanesulfonamide (Compound 135, 18 mg, 20.64 mol, 7% yield) as an off-white solid. UPLC-MS (ES+): m/z 870.6 [M+H]⁺. ¹H NMR (400 MHz, DMSO-d₆): δ 10.79 (s, 1H), 9.02 (bs, 1H), 8.31 (s, 1H), 7.76 (d, J=8.80 Hz, 1H), 7.64 (dd, J=3.20, 8.80 Hz, 1H), 7.40-7.29 (m, 3H), 7.01-6.94 (m, 1H), 6.50-6.38 (m, 2H), 6.02 (d, J=8.00 Hz, 1H), 5.36-5.21 (m, 1H), 4.36-4.26 (m, 1H), 4.23-4.05 (m, 2H), 3.20-2.90 (m, 3H), 2.83-2.72 (m, 1H), 2.68-2.61 (m, 2H), 2.44-2.37 (m, 1H), 2.28-2.00 (m, 6H), 1.98-1.79 (m, 11H), 1.76-1.68 (m, 3H), 1.61-1.30 (m, 6H).

Similarly, the second eluted fraction (peak-2) was concentrated under reduced pressure at 40° C. and lyophilized to afford Compound 136 (assigned as stereoisomer 2).

Peak-2: N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]cyclopentanesulfonamide (Compound 136, 45 mg, 50.87 mol, 17% yield) as an off-white solid. UPLC-MS (ES+): m/z 870.2 [M+H]⁺. ¹H NMR (400 MHz, DMSO-d₆): δ 10.77 (s, 1H), 8.59 (s, 1H), 8.32 (s, 1H), 7.76 (d, J=8.80 Hz, 1H), 7.64 (dd, J=2.80, 8.80 Hz, 1H), 7.55-7.19 (m, 3H), 7.18-6.99 (m, 1H), 6.54-6.41 (m, 2H), 6.00 (d, J=7.20 Hz, 1H), 5.35-5.18 (m, 1H), 4.39-4.22 (m, 1H), 4.21-4.01 (m, 2H), 3.59-3.41 (m, 1H), 3.18-2.70 (m, 4H), 2.61-2.58 (m, 1H), 2.44-2.38 (m, 1H), 2.27-1.41 (m, 26H).

### Synthesis D72: Synthesis of 6-[2-cyano-3-[[ethyl(methyl)sulfamoyl]amino]-6-fluoro-phenoxy]-3-[2-[1-[4-[3-(2,4-dioxohexahydropyrimidin-1-yl)-5-fluoro-1-methyl-indazol-6-yl]cyclohexyl]-4-piperidyl]ethyl]-4-oxo-quinazoline (Compound 137 and Compound 138)

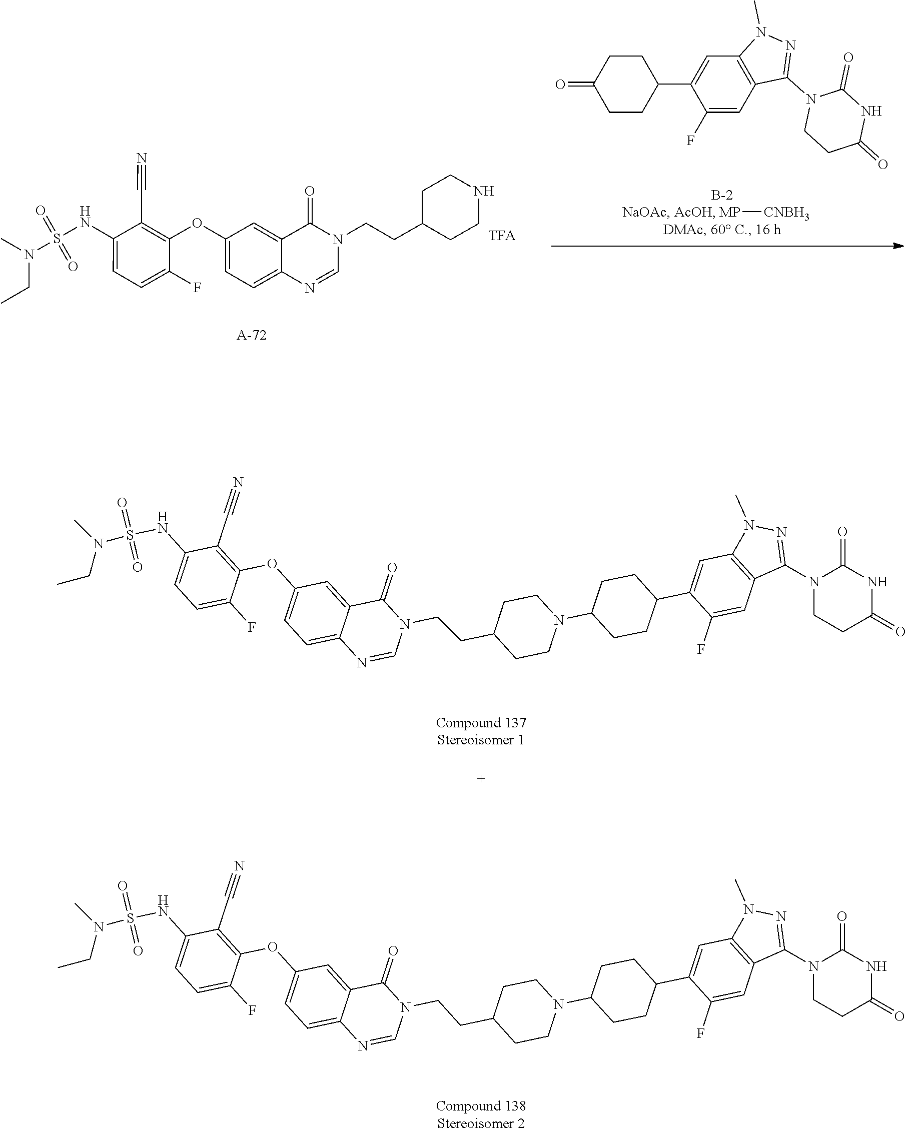

Into a 20 mL pressure vial containing a well stirred solution of 6-[2-cyano-3-[[ethyl(methyl)sulfamoyl]amino]-6-fluoro-phenoxy]-4-oxo-3-[2-(4-piperidyl)ethyl]quinazoline (A-72) (500 mg, 778.06 mol, TFA salt) in DMAc (5 mL), was added NaOAc (127.65 mg, 1.56 mmol) at room temperature and stirred for 15 minutes at the same temperature. After 15 minutes of stirring, 1-[5-fluoro-1-methyl-6-(4-oxocyclohexyl)indazol-3-yl]hexahydropyrimidine-2,4-dione (B-2) (334.60 mg, 933.68 mol) and AcOH (2.33 mmol, 133.49 μL) were added at 0° C. and stirring was continued at room temperature for 8 hours. Subsequently, MP-CNBH₃ (500 mg, 778.06 mol) was added and resulting reaction mixture was stirred at 60° C. for 16 hours. After completion of the reaction, the reaction mixture was filtered through cotton pad and filtrate was concentrated under reduced pressure to get a crude residue which was purified by reverse phase column chromatography (Column: Redisep R_{f} Gold® reversed-phase, C18-Teledyne ISCO, 120 g), Mobile phase A: 10 mM NH₄HCO₃ in milli-Q water; Mobile phase B: acetonitrile). Fractions were collected and lyophilized to afford 250 mg of a mixture of cis and trans isomers. Further cis and trans isomers were separated by preparative HPLC [Column: X-Bridge C18 (150 mm*21 mm*5 m), Mobile phase A: 10 mM NH₄HCO₃ in milli-Q water (pH was adjusted to 10 with aqueous ammonia); Mobile phase B: acetonitrile]. The first eluting fraction (Peak-1) was lyophilized to afford Compound 137 (assigned as stereoisomer 1).

Peak-1: 6-[2-cyano-3-[[ethyl(methyl)sulfamoyl]amino]-6-fluoro-phenoxy]-3-[2-[1-[4-[3-(2,4-dioxohexahydropyrimidin-1-yl)-5-fluoro-1-methyl-indazol-6-yl]cyclohexyl]-4-piperidyl]ethyl]-4-oxo-quinazoline (Compound 137, 24.89 mg, 28.50 mol, 4% yield) as an off-white solid. UPLC-MS (ES+): m/z 871.6 [M+H]⁺. ¹H NMR (400 MHz, DMSO-d₆): δ 10.53 (s, 1H), 8.79 (bs, 1H), 8.38 (s, 1H), 7.76 (d, J=9.20 Hz, 1H), 7.63 (dd, J=2.80, 9.00 Hz, 1H), 7.53 (d, J=5.60 Hz, 1H), 7.40-7.20 (m, 4H), 4.04-3.99 (m, 6H), 3.91 (t, J=6.40 Hz, 2H), 3.10-2.85 (m, 6H), 2.77 (t, J=6.40 Hz, 2H), 2.60-2.54 (m, 3H), 2.20-2.09 (m, 2H), 2.08-1.96 (m, 4H), 1.80-1.30 (m, 10H), 1.03 (t, J=7.20 Hz, 3H).

Similarly, the second eluted fraction was lyophilized to afford Compound 138 (assigned as stereoisomer 2).

Peak-2: 6-[2-cyano-3-[[ethyl(methyl)sulfamoyl]amino]-6-fluoro-phenoxy]-3-[2-[1-[4-[3-(2,4-dioxohexahydropyrimidin-1-yl)-5-fluoro-1-methyl-indazol-6-yl]cyclohexyl]-4-piperidyl]ethyl]-4-oxo-quinazoline (Compound 138, 31.39 mg, 34.98 mol, 5% yield) as an off-white solid. UPLC-MS (ES+): m/z 871.5 [M+H]⁺. ¹H NMR (400 MHz, DMSO-d₆): δ 10.56 (s, 1H), 8.48 (bs, 1H), 8.38 (s, 1H), 7.76 (d, J=8.80 Hz, 1H), 7.63 (dd, J=2.80, 9.00 Hz, 1H), 7.48 (d, J=5.60 Hz, 1H), 7.41-7.28 (m, 4H), 4.07-3.95 (m, 5H), 3.91 (t, J=6.80 Hz, 2H), 3.65-3.48 (m, 1H), 3.28-3.10 (m, 2H), 3.09-2.80 (m, 4H), 2.76 (t, J=6.80 Hz, 2H), 2.58 (s, 3H), 2.20-1.60 (m, 13H), 1.59-1.25 (m, 3H), 1.03 (t, J=7.20 Hz, 3H).

### Synthesis D73: Synthesis of (3S)-N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 139 and Compound 140)

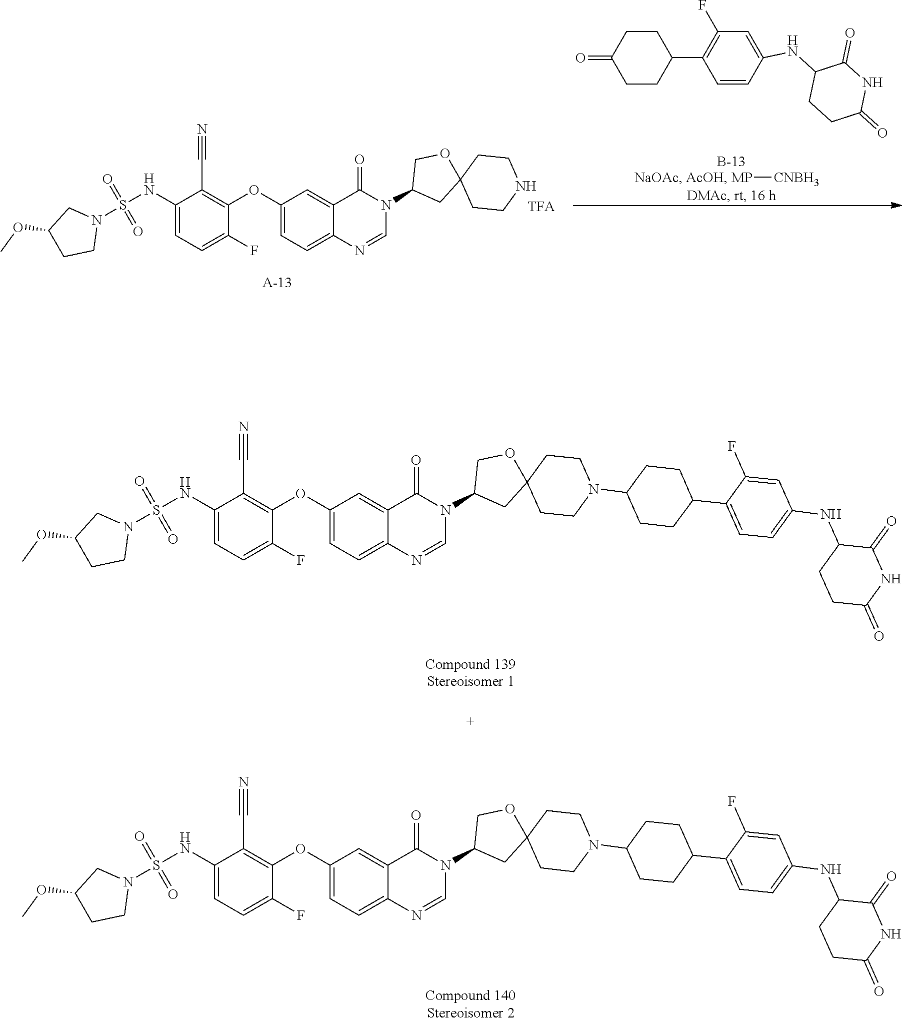

Into a 20 mL pressure vial containing a well stirred solution of (3S)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-13) (360 mg, 505.14 mol, TFA salt) and anhydrous NaOAc (124.31 mg, 1.52 mmol) in DMAc (5.02 mL) at room temperature and resulting mixture was stirred for 30 min at the same temperature. 3-[3-fluoro-4-(4-oxocyclohexyl)anilino]piperidine-2,6-dione (B-13) (241.21 mg, 757.72 mol) and AcOH (2.53 mmol, 144.44 μL) were added at 0° C. and stirred for 3 h at room temperature. Subsequently, MP-CNBH₃ (200 mg, 505.14 mol) was added, and the resulting reaction mixture was stirred at room temperature for 16 hours. After completion of the reaction, the reaction mixture was filtered and the filtrate was concentrated under reduced pressure to get a crude residue which was purified by reverse phase column chromatography (Column: Redisep R_{f} Gold® reversed-phase C18-teledyne ISCO, 120 g), [Mobile phase A: NH₄HCO₃ in milli-Q water; Mobile phase B: acetonitrile). Fractions were collected and lyophilized to afford 120 mg of a mixture of cis and trans isomers. Further cis and trans isomers were separated by preparative HPLC [Column: X-Bridge C18 (250 mm*19 mm*5 m), Mobile phase A: 10 mM NH₄HCO₃ and NH₄OH (pH-10); Mobile phase B: 100% acetonitrile, Flow rate: 14 mL/min]. First eluting fraction at t_{R}=4.70 min was concentrated under reduced pressure at 40° C. and lyophilized to afford Compound 139 (assigned as stereoisomer 1).

Peak-1: (3S)-N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 139, 12.7 mg, 13.99 mol, 3% yield) as an white solid. UPLC-MS (ES+): m/z 901.6 [M+H]⁺. ¹H NMR (400 MHz, DMSO-d₆): δ 10.79 (s, 1H), 9.00 (bs, 1H), 8.31 (s, 1H), 7.76 (d, J=8.80 Hz, 1H), 7.64 (dd, J=3.20, 9.00 Hz, 1H), 7.34-7.30 (m, 3H), 6.97 (t, J=8.80 Hz, 1H), 6.46-6.43 (m, 2H), 6.02 (d, J=7.60 Hz, 1H), 5.31-5.24 (m, 1H), 4.34-4.28 (m, 1H), 4.22-4.09 (m, 2H), 3.95-3.92 (m, 1H), 3.26 (t, J=6.00 Hz, 2H), 3.18 (s, 3H), 3.15-2.98 (m, 5H), 2.74-2.70 (m, 1H), 2.63-2.59 (m, 2H), 2.38-3.40 (m, 1H), 2.20-1.70 (m, 13H), 1.65-1.50 (m, 4H).

Similarly, the second eluted fraction at t_{R}=4.93 min was concentrated under reduced pressure at 40° C. and lyophilized to afford Compound 140 (assigned as stereoisomer 2).

Peak-2: (3S)-N-[2-cyano-3-[3-[(3R)-8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 140, 22.3 mg, 24.49 mol, 5% yield) as an white solid. UPLC-MS (ES+): m/z 901.5 [M+H]⁺. ¹H NMR (400 MHz, DMSO-d₆): δ 10.79 (s, 1H), 8.48 (bs, 1H), 8.32 (s, 1H), 7.76 (d, J=8.80 Hz, 1H), 7.64 (dd, J=2.80, 9.00 Hz, 1H), 7.43-7.28 (m, 3H), 7.15-7.05 (m, 1H), 6.53-6.44 (m, 2H), 6.06-5.99 (m, 1H), 5.34-5.25 (m, 1H), 4.34-4.28 (m, 1H), 4.22-4.08 (m, 2H), 3.98-3.92 (m, 1H), 3.62-3.42 (m, 3H), 3.18 (s, 3H), 3.15-2.90 (m, 5H), 2.79-2.70 (m, 1H), 2.40-2.37 (m, 2H), 2.26-1.60 (m, 17H).

### Synthesis D74: Synthesis of 6-[2-Cyano-3-[[ethyl(methyl)sulfamoyl]amino]-6-fluoro-phenoxy]-3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazoline (Compound 141)

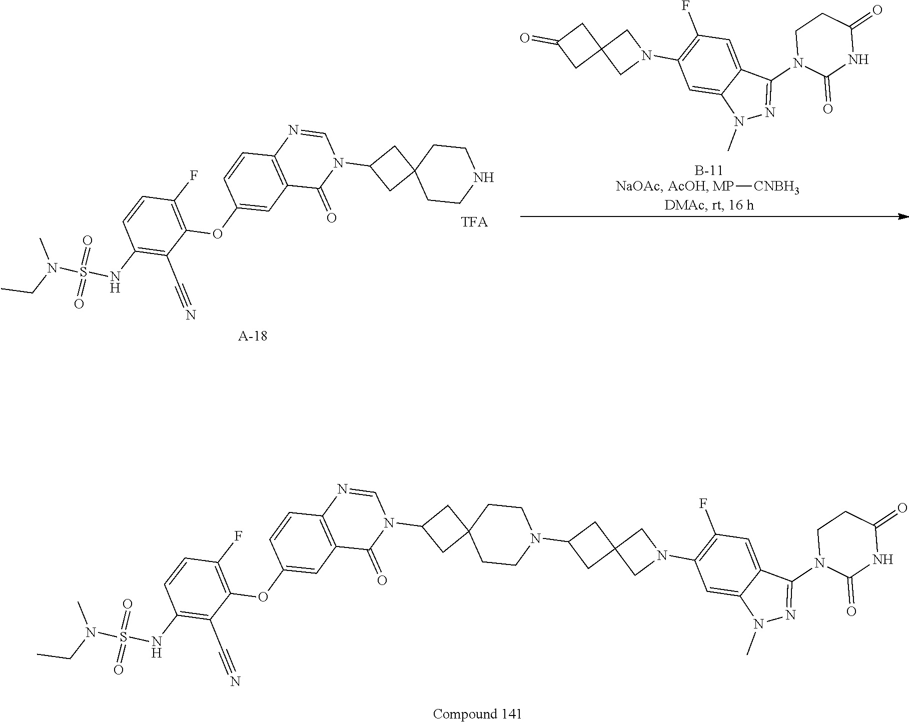

Into a 20 mL pressure capped vial containing a well-stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (120 mg, 188 μmol, TFA salt) in anhydrous DMAc (3.01 mL) was added NaOAc (63.10 mg, 769.26 μmol) and stirred for 10 minutes. To the above mixture, 1-[5-fluoro-1-methyl-6-(6-oxo-2-azaspiro[3.3]heptan-2-yl)indazol-3-yl]hexahydropyrimidine-2,4-dione (B-11) (96.29 mg, 260 μmol) and AcOH (461.55 μmol, 26.4 μL) were added at room temperature and mixture was stirred for 30 minutes. Finally, MP-CNBH₃ (120 mg, 153.85 μmol) was added. The resulting mixture was stirred at room temperature for 16 hours. After completion of the reaction, the reaction mixture was filtered through cotton pad and filtrate was concentrated under reduced pressure to get a crude residue which was purified by reverse phase [Column: Redisep R_{f}Gold® reversed-phase C18-teledyne ISCO, 120 g; Mobile phase A: 10 mM NH₄CO₃ in milli-Q water; Mobile phase B: acetonitrile] to afford 6-[2-cyano-3-[[ethyl(methyl)sulfamoyl]amino]-6-fluoro-phenoxy]-3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazoline (Compound 141, 35 mg, 38.51 μmol, 21% yield) as an off-white solid. UPLC-MS (ES+): m/z 896.6 [M+H]⁺. ¹H NMR (400 MHz, DMSO-d₆): δ 10.51 (s, 1H), 9.50 (bs, 1H), 8.39 (s, 1H), 7.77 (d, J=9.20 Hz, 1H), 7.64 (dd, J=2.80, 8.80 Hz, 1H), 7.55-7.45 (m, 1H), 7.34-7.26 (m, 3H), 6.52 (d, J=7.60 Hz, 1H), 5.00-4.91 (m, 1H), 3.86-4.05 (m, 7H), 3.49-3.41 (m, 2H), 3.31-3.21 (m, 2H), 3.10-2.90 (m, 2H), 2.73 (t, J=6.80 Hz, 2H), 2.68-2.64 (m, 2H), 2.45-2.22 (m, 8H), 2.10-1.50 (m, 4H), 1.04 (t, J=7.20 Hz, 3H).

### Synthesis D75: Synthesis of (3R)-N-[2-cyano-3-[(3S)-3-[8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 142 and Compound 143)

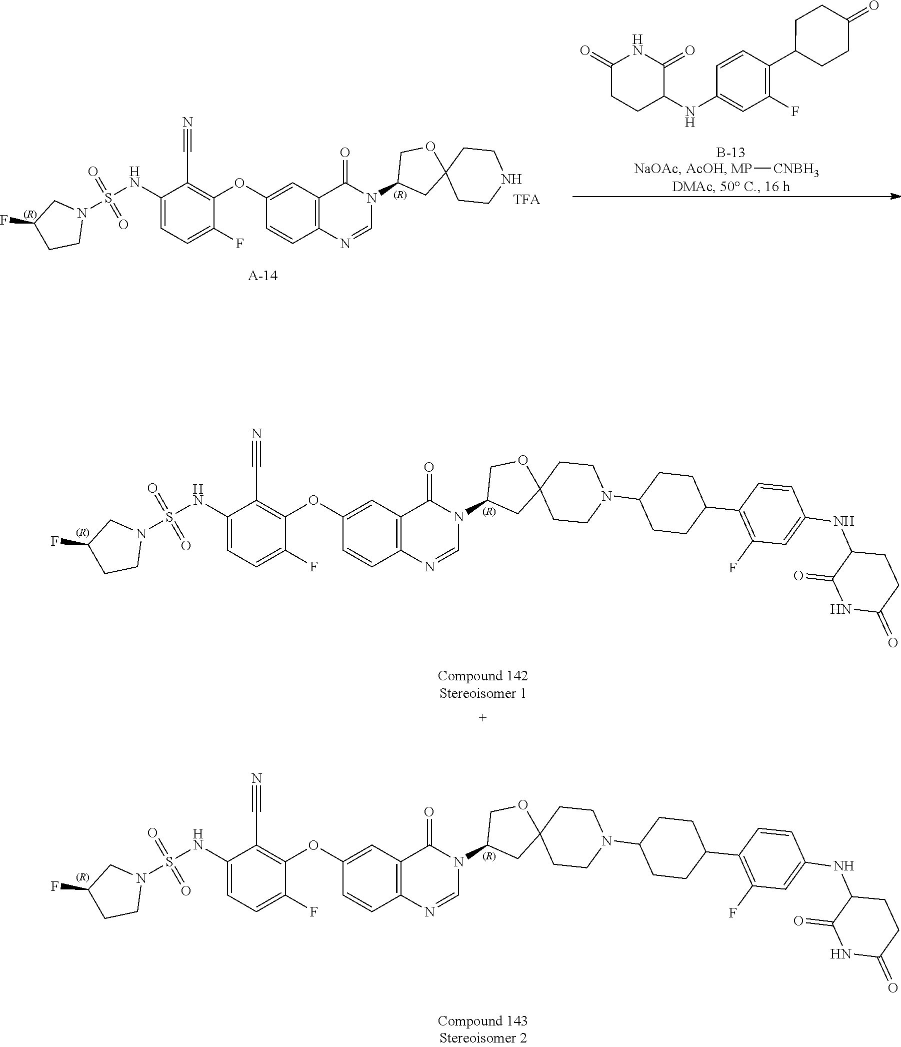

Into a 20 mL pressure vial containing a well-stirred solution of (3R)-N-[2-cyano-4-fluoro-3-[(3S)-3-(1-oxa-8-azaspiro[4.5]decan-3-yl)-4-oxo-quinazolin-6-yl]oxy-phenyl]-3-fluoro-pyrrolidine-1I-sulfonamide (A-14) (500 mg, 649.41 mol, TFA salt) in DMAc (5 mL) was added anhydrous NaOAc (159.81 mg, 1.95 mmol) at room temperature under nitrogen atmosphere and mixture was stirred for 15 min. Reaction mixture was cooled to 0° C. and was added 3-[3-fluoro-4-(4-oxocyclohexyl)anilino]piperidine-2,6-di one (B-13) (206.74 mg, 649.41 mol) followed by AcOH (974.12 mol, 55.71 μL) and resulting mixture was stirred for 3 h at room temperature. Finally, MP-CNBH₃ (750 mg, 649.41 mol) was added, and the reaction mixture was stirred at 50° C. for 16 hours. After the completion of the reaction, the reaction mixture was filtered and filtrate was concentrated under reduced pressure to get a crude residue which was purified by reverse phase column chromatography (Redisep R_{f} Gold®reversed-phase C18-teledyne ISCO, 120 g), [Mobile phase A: 10 mM NH₄HCO₃ in milli-Q water; Mobile phase B: acetonitrile]. The fractions were lyophilized to afford 350 mg of mixture of cis and trans isomers. Further cis and trans isomers were separated by preparative HPLC (Column: X BRIDGE C18 (150 mm*21 mm*5 μm), [Mobile phase A: NH₄HCO₃ in milli-Q water (pH adjusted to 10 with ammonia); Mobile phase B: acetonitrile]. The first eluting fraction (Peak-1) was concentrated under reduced pressure at 40° C. and lyophilized to afford Compound 142 (assigned as stereoisomer 1).

Peak 1: (3R)-N-[2-cyano-3-[(3S)-3-[8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 142, 2.16 mg, 2.42 mol, 4% yield) as an off-white solid. UPLC-MS (ES+): m/z 889.5 [M+H]⁺. ¹H NMR (400 MHz, DMSO-d₆): δ 10.78 (s, 1H), 9.01 (s, 1H), 8.31 (s, 1H), 7.75 (d, J=9.20 Hz, 1H), 7.63 (dd, J=2.80, 8.80 Hz, 1H), 7.34-7.28 (m, 3H), 6.97 (t, J=8.40 Hz, 1H), 6.51-6.43 (m, 2H), 6.01 (d, J=7.60 Hz, 1H), 5.38-5.17 (m, 2H), 4.40-4.29 (m, 1H), 4.24-4.09 (m, 2H), 3.42-3.38 (m, 1H), 3.23-2.98 (m, 5H), 2.80-2.71 (m, 1H), 2.64-2.58 (m, 2H), 2.43-2.38 (m, 1H), 2.30-1.70 (m, 16H), 1.62-1.50 (m, 4H).

Similarly, the second eluted fraction (Peak-2) was concentrated under reduced pressure at 40° C. and lyophilized to afford Compound 143 (assigned as stereoisomer 2).

Peak-2: (3R)-N-[2-cyano-3-[(3S)-3-[8-[4-[4-[(2,6-dioxo-3-piperidyl)amino]-2-fluoro-phenyl]cyclohexyl]-1-oxa-8-azaspiro[4.5]decan-3-yl]-4-oxo-quinazolin-6-yl]oxy-4-fluoro-phenyl]-3-fluoro-pyrrolidine-1-sulfonamide (Compound 143, 4.40 mg, 4.94 mol, 8% yield) as an off-white solid. UPLC-MS (ES+): m/z 889.6 [M+H]⁺. ¹H NMR (400 MHz, DMSO-d₆): δ 10.78 (s, 1H), 8.49 (s, 1H), 8.31 (s, 1H), 7.76 (d, J=8.80 Hz, 1H), 7.63 (dd, J=2.80, 8.80 Hz, 1H), 7.41-7.31 (m, 3H), 7.10 (s, 1H), 6.50-6.44 (m, 2H), 6.02 (d, J=7.60 Hz, 1H), 5.40-5.12 (m, 2H), 4.39-4.28 (m, 1H), 4.23-4.07 (m, 2H), 3.67-3.42 (m, 2H), 3.22-2.85 (m, 6H), 2.78-2.69 (m, 1H), 2.62-2.58 (m, 1H), 2.48-2.40 (m, 1H), 2.28-1.49 (m, 19H).

### Synthesis D76: Synthesis of (3R)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 144)

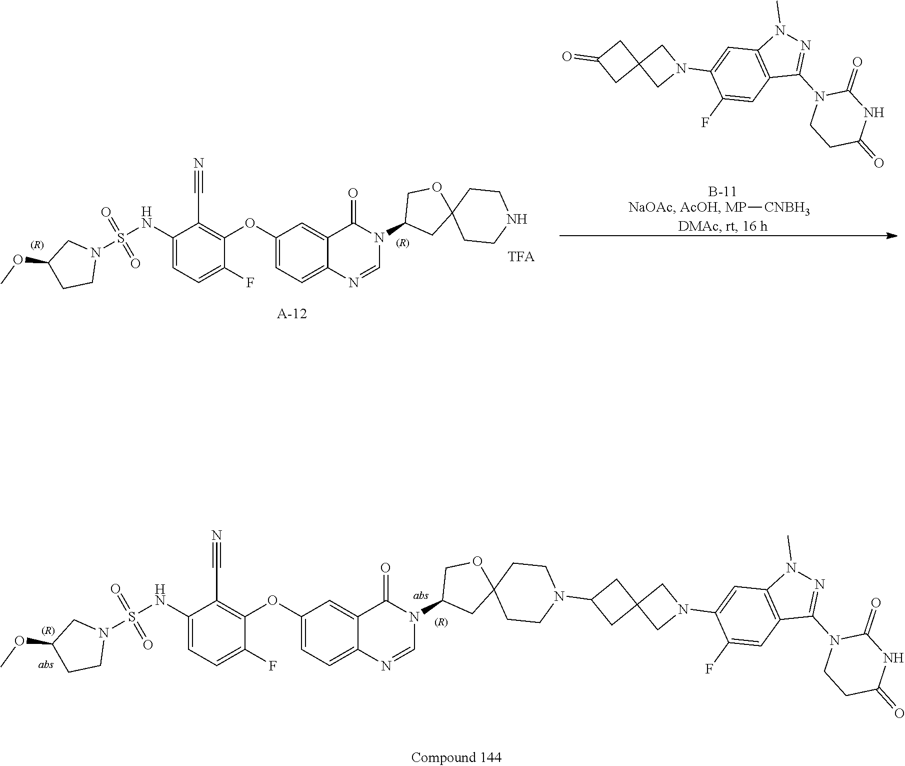

Into a 8 mL pressure vial containing a well-stirred solution of (3R)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-12) (161.71 mg, 207.67 mol, TFA salt) in DMAc (4 mL) was added anhydrous NaOAc (51.11 mg, 623.01 mol) at room temperature under nitrogen atmosphere and mixture was stirred for 30 min. Reaction mixture was cooled to 0° C. and was added 1-[5-fluoro-1-methyl-6-(6-oxo-2-azaspiro[3.3]heptan-2-yl)indazol-3-yl]hexahydropyrimidine-2,4-dione (B-11) (85.29 mg, 207.67 mol) followed by AcOH (1.04 mmol, 59.38 μL). The resulting mixture was stirred for 3 h at room temperature. Finally, MP-CNBH₃ (148 mg, 207.67 mol) was added, and the reaction mixture was stirred at room temperature for 16 hours. After the completion of the reaction, the reaction mixture was filtered and the filtrate was concentrated under reduced pressure to get a crude residue which was purified by reverse phase column chromatography (Redisep R_{f} Gold®reversed-phase C18-teledyne ISCO, 120 g), [Mobile phase A: NH₄HCO₃ in milli-Q water; Mobile phase B: acetonitrile]. The fractions were lyophilized to afford 45 mg of (3R)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-methoxy-pyrrolidine-1-sulfonamide, which is impure. Further separated by preparative HPLC [Column: X-Bridge C8 (250 mm*19.5 mm*5 m), Mobile phase A: 10 mm NH₄HCO₃ in milli-Q water; Mobile phase B: acetonitrile]. The eluting fraction at t_{R}=12.7 min was concentrated under reduced pressure at 40° C. and lyophilized to afford (3R)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 144, 13.01 mg, 13.21 mol, 6% yield) as a white solid. UPLC-MS (ES+): m/z 954.7 [M+H]⁺. ¹H NMR (400 MHz, DMSO-d₆): δ 10.50 (s, 1H), 9.57 (s, 1H), 8.31 (s, 1H), 7.76 (d, J=8.80 Hz, 1H), 7.65 (dd, J=2.80 Hz, 9.2 Hz, 1H), 7.50 (bs, 1H), 7.35-7.34 (m, 2H), 7.27 (d, J=12.80 Hz, 1H), 6.51 (d, J=7.60 Hz, 1H), 5.30 (q, J=5.60 Hz, 1H), 4.20-4.07 (m, 2H), 4.06-4.00 (m, 2H), 3.85 (s, 3H), 3.84-3.80 (m, 5H), 3.32-3.28 (m, 2H), 3.24-3.01 (m, 8H), 2.72 (t, J=6.40 Hz, 3H), 2.45-2.37 (m, 3H), 2.32-2.09 (m, 3H), 2.04-1.57 (m, 7H).

### Synthesis D77: Synthesis of 6-[2-Cyano-6-fluoro-3-[[2-hydroxyethyl(methyl)sulfamoyl]amino]phenoxy]-3-[7-[4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 145 and Compound 146)

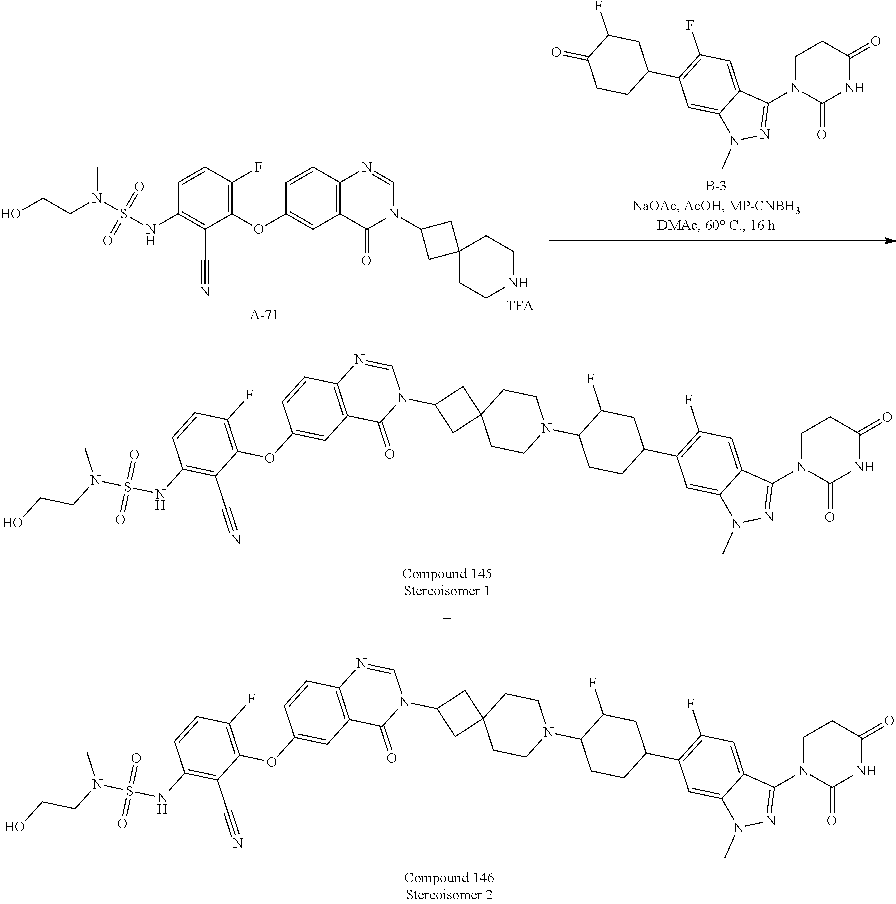 [SMILES: CN(CCO)S(=O)(=O)Nc1ccc(F)c(Oc2ccc3ncn(C4CC5(CCNCC5)C4)c(=O)c3c2)c1C#N ; Cn1nc(N2CCC(=O)NC2=O)c2cc(F)c(C3CCC(=O)C(F)C3)cc21 ; "A-71" ; CN(CCO)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N ; CN(CCO)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N]

Into an 20 mL pressure vial containing a well stirred solution of 3-(7-azaspiro[3.5]nonan-2-yl)-6-[2-cyano-6-fluoro-3-[[2-hydroxyethyl(methyl)sulfamoyl]amino]phenoxy]-4-oxo-quinazoline (A-71) (0.250 g, 372.78 mol, TFA salt) and 1-[5-fluoro-6-(3-fluoro-4-oxo-cyclohexyl)-1-methyl-indazol-3-yl]hexahydropyrimidine-2,4-dione (B-3) (210.45 mg, 559.17 mol) in DMAc (5.0 mL) was added NaOAc (152.90 mg, 1.86 mmol) at room temperature and stirred for 30 min. After 30 min of stirring, AcOH (1.12 mmol, 63.96 μL) was added and stirring was continued at room temperature for 2 hours. Subsequently, MP-CNBH₃ (0.375 g, 372.78 mol) was added and resulting reaction mixture was stirred at 60° C. for 16 hours. After completion of the reaction, the reaction mixture was filtered through a cotton pad and filtrate was concentrated under reduced pressure to get a crude residue which was purified by preparative HPLC [Column: X-BRIDGE C18 (150 mm*19 mm*5 μm), Mobile phase A: 10 mM NH₄HCO₃ (pH was adjusted to 10 with ammonia) in H₂O, Mobile phase B: 100% acetonitrile, flow:15 mL/min). First eluting fraction (Peak-1) was lyophilized to afford Compound 145 (assigned as stereoisomer 1).

Peak-1: 6-[2-cyano-6-fluoro-3-[[2-hydroxyethyl(methyl)sulfamoyl]amino]phenoxy]-3-[7-[4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 145, 0.019 g, 20.47 mol, 5% yield) as a white solid. UPLC-MS (ES+): m/z 917.5 [M+H]⁺. ¹H NMR (400 MHz, DMSO-d₆): δ 10.56 (s, 1H), 9.75 (bs, 1H), 8.40 (s, 1H), 7.77 (d, J=8.80 Hz, 1H), 7.65-7.51 (m, 3H), 7.41-7.36 (m, 3H), 5.50-5.25 (m, 1H), 5.00-4.50 (m, 2H), 4.00 (s, 3H), 3.90 (t, J=6.80 Hz, 2H), 3.50 (t, J=6.00 Hz, 2H), 3.28-3.18 (m, 1H), 3.15-2.70 (m, 11H), 2.41-2.30 (m, 4H), 2.26-2.17 (m, 1H), 2.08-1.60 (m, 10H).

Similarly, second eluted fraction (Peak-2) was lyophilized to afford Compound 146 (assigned as stereoisomer 2).

Peak-2: 6-[2-cyano-6-fluoro-3-[[2-hydroxyethyl(methyl)sulfamoyl]amino]phenoxy]-3-[7-[4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-4-oxo-quinazoline (Compound 146, 0.015 g, 16.17 mol, 4% yield) as a white solid. UPLC-MS (ES+): m/z 917.6 [M+H]⁺. ¹H NMR (400 MHz, DMSO-d₆): δ 10.56 (s, 1H), 10.06 (bs, 1H), 8.41 (s, 1H), 7.79-7.77 (m, 2H), 7.66 (dd, J=3.20, 9.00 Hz, 1H), 7.52 (d, J=6.00 Hz, 1H), 7.46-7.37 (m, 3H), 5.30-5.00 (m, 1H), 4.99-4.88 (m, 1H), 4.87-4.70 (m, 1H), 4.03 (s, 3H), 3.91 (t, J=6.80 Hz, 2H), 3.51 (t, J=6.00 Hz, 2H), 3.20-3.10 (m, 2H), 2.90-2.73 (m, 6H), 2.30-2.20 (m, 3H), 2.18-1.63 (m, 11H).

### Synthesis D78: Synthesis of 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]methyl]quinoxaline (Compound 147 and Compound 148)

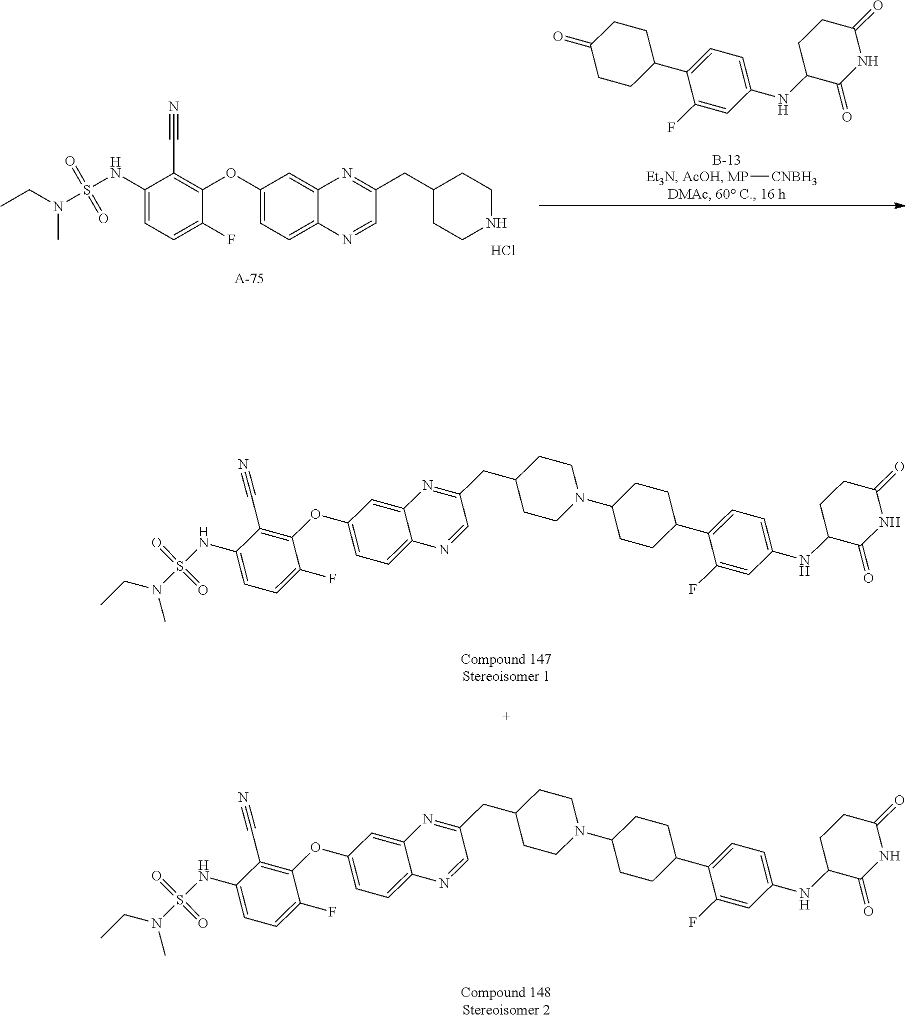

Into a 20 mL glass vial with pressure released cap containing a well-stirred solution of 7-[2-cyano-3-[[ethyl(methyl)sulfamoyl]amino]-6-fluoro-phenoxy]-2-(4-piperidylmethyl)quinoxaline (A-75) (260 mg, 476.72 mol, HCl salt) in DMAc (4 mL) was added triethylamine (2.38 mmol, 332.23 μL) and stirred at room temperature for 10 min. Subsequently, AcOH (1.91 mmol, 109.06 μL) and 3-[3-fluoro-4-(4-oxocyclohexyl)anilino]piperidine-2,6-dione (B-13) (183.95 mg, 572.06 mol) were added and stirred at room temperature for 2 hours. MP-CNBH₃ (260 mg) was added, and the reaction mixture was stirred at 60° C. for 16 hours. After completion of the reaction, the reaction mixture was filtered and filtrate was concentrated under reduced pressure to get a crude residue which was purified by preparative HPLC [Column: X-Bridge C18 (150 mm*19 mm*5 μm), Mobile phase A: 10 mM NH₄HCO₃ & NH₄OH_pH-10 in milli-Q water; Mobile phase B: acetonitrile]. First eluting fraction (Peak-1) was lyophilized to afford Compound 147 (assigned as stereoisomer 1).

Peak-1: 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]methyl]quinoxaline (Compound 147.31 mg, 38.59 mol, 8% yield) as an off-white solid. UPLC-MS (ES+): m/z 801.5 [M+H]⁺. ¹H NMR (400 MHz, DMSO-d₆): δ 10.79 (s, 1H), 8.81 (m, 2H), 8.13 (d, J=9.20 Hz, 1H), 7.70 (dd, J=2.80, 9.20 Hz, 1H), 7.38-7.36 (m, 1H), 7.29-7.26 (m, 1H), 7.07-7.06 (m, 1H), 6.98-6.94 (m, 1H), 6.46-6.42 (m, 2H), 6.03-6.01 (m, 1H), 4.32-4.28 (m, 1H), 3.49-3.37 (m, 2H), 3.32-3.11 (m, 1H), 3.02-2.94 (m, 6H), 2.74-2.60 (m, 1H), 2.59-2.52 (m, 5H), 2.32-2.03 (m, 4H), 1.92-1.81 (m, 5H), 1.54-1.48 (m, 6H), 1.04 (t, J=7.20 Hz, 3H).

Similarly, the second eluted fraction (Peak-2) was lyophilized to afford Compound 148 (assigned as stereoisomer 2).

Peak-2: 7-[2-cyano-3-[[ethyl(methyl)sulfamoyl]amino]-6-fluoro-phenoxy]-2-[[1-[4-[4-[(2,6-dioxo-3-piperidyl)amino]-2-fluoro-phenyl]cyclohexyl]-4-piperidyl]methyl]quinoxaline (Compound 148.26 mg, 31.89 mol, 7% yield) as an off-white solid. UPLC-MS (ES+): m/z 801.5 [M+H]⁺. ¹H NMR (400 MHz, DMSO-d₆): δ 10.79 (s, 1H), 8.81 (m, 2H), 8.13 (d, J=9.20 Hz, 1H), 7.70 (dd, J=2.80, 9.20 Hz, 1H), 7.20-7.50 (m, 2H), 6.99-7.19 (m, 2H), 6.46-6.42 (m, 2H), 6.03-6.01 (m, 1H), 4.32-4.28 (m, 1H), 3.55-3.45 (m, 1H), 3.30-3.19 (m, 1H), 3.09-2.83 (m, 7H), 2.82-2.74 (m, 1H), 2.61-2.57 (m, 4H), 2.29-2.01 (m, 2H), 1.40-2.01 (m, 14H), 1.04 (t, J=7.20 Hz, 3H).

### Synthesis D79: Synthesis of 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]methyl]quinoxaline (Compound 149 and Compound 150)

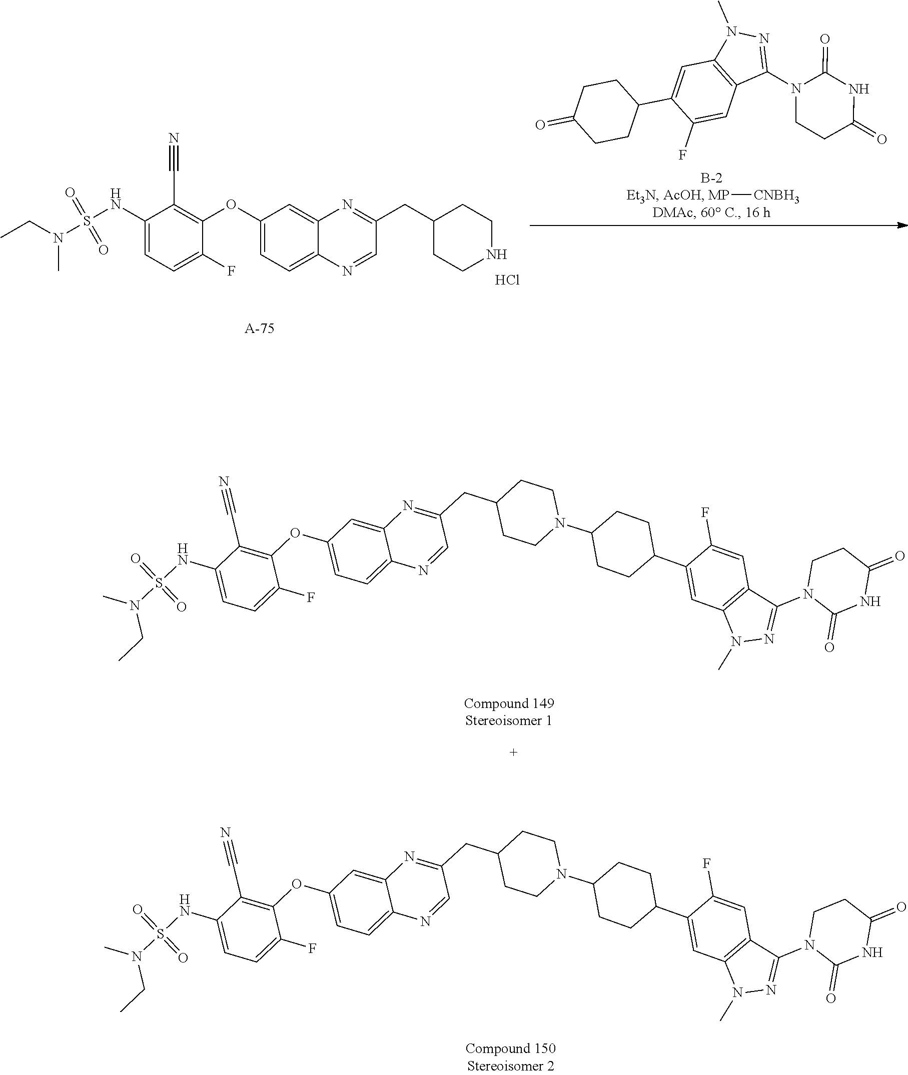

Into a 20 mL glass vial with pressure released cap containing a well-stirred solution of 7-[2-cyano-3-[[ethyl(methyl)sulfamoyl]amino]-6-fluoro-phenoxy]-2-(4-piperidylmethyl)quinoxaline (A-75) (340 mg, 622.77 mol, HCl salt) in DMAc (6 mL) was added triethylamine (3.11 mmol, 434.01 μL) at room temperature and stirred for 10 min. Subsequently, AcOH (2.49 mmol, 142.47 μL) and 1-[5-fluoro-1-methyl-6-(4-oxocyclohexyl)indazol-3-yl]hexahydropyrimidine-2,4-dione (B-2) (267.81 mg, 747.32 mol) was added and stirred at room temperature for 2 hours. Finally, MP-CNBH₃ (340 mg) was added, and the reaction mixture was stirred at 60° C. for 16 hours. After the completion of the reaction, the reaction mixture was filtered and filtrate was concentrated under reduced pressure to get a crude residue which was purified by preparative HPLC [Column: X-Bridge C8 (150 mm*19 mm*5 μm), Mobile phase A: 10 mM NH₄HCO₃ & NH₄OH_pH-10 in milli-Q water; Mobile phase B: acetonitrile]. First eluting fraction (Peak-1) was lyophilized to afford Compound 149 (assigned as stereoisomer 1).

Peak-1: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]methyl]quinoxaline (Compound 149, 76 mg, 90.26 mol, 14% yield) as an off-white solid.UPLC-MS (ES+): m/z 841.5 [M+H]⁺. ¹H NMR (400 MHz, DMSO-d₆): δ 10.55 (m, 1H), 8.81-8.79 (m, 1H), 8.15-8.10 (m, 1H), 7.71-7.66 (m, 1H), 7.53 (d, J=6.00 Hz, 1H), 7.39-7.34 (m, 2H), 7.28-7.25 (m, 1H), 7.07 (d, J=2.80 Hz, 1H), 3.99-3.92 (m, 3H), 3.89 (t, J=6.40 Hz, 2H), 3.33-3.28 (m, 2H), 3.26-2.86 (m, 8H), 2.75 (t, J=6.80 Hz, 2H), 2.68-2.67 (m, 3H), 2.56-2.55 (m, 1H), 2.50-1.99 (m, 5H), 1.97-1.82 (m, 1H), 1.68-1.46 (m, 6H), 1.03 (t, J=7.20 Hz, 3H).

Similarly, the second eluted fraction (Peak-2) was lyophilized to afford Compound 150 (assigned as stereoisomer 2). Peak-2: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-piperidyl]methyl]quinoxaline (Compound 150, 74 mg, 85.74 mol, 14% yield) as an off-white solid. UPLC-MS (ES+): m/z 841.5 [M+H]⁺. ¹H NMR (400 MHz, DMSO-d₆): δ 10.56 (s, 1H), 8.81 (s, 1H), 8.15-8.13 (m, 1H), 7.70 (dd, J=2.80, 9.20 Hz, 1H), 7.47 (d, J=6.00 Hz, 1H), 7.41 (d, J=11.20 Hz, 2H), 7.40-7.28 (m, 1H), 7.21-7.00 (m, 1H), 4.02-4.00 (m, 3H), 3.91 (t, J=6.40 Hz, 2H), 3.50-3.43 (m, 2H), 3.33-3.14 (m, 1H), 3.04-2.95 (m, 5H), 2.76 (t, J=6.80 Hz, 2H), 2.68-2.59 (m, 3H), 2.57-2.57 (m, 2H), 2.34-1.99 (m, 6H), 1.96-1.78 (m, 5H), 1.55-1.46 (m, 2H), 1.04 (t, J=7.20 Hz, 3H).

### Synthesis D80: Synthesis of 6-[2-cyano-6-fluoro-3-[[2-hydroxyethyl(methyl)sulfamoyl]amino]phenoxy]-3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazoline (Compound 151)

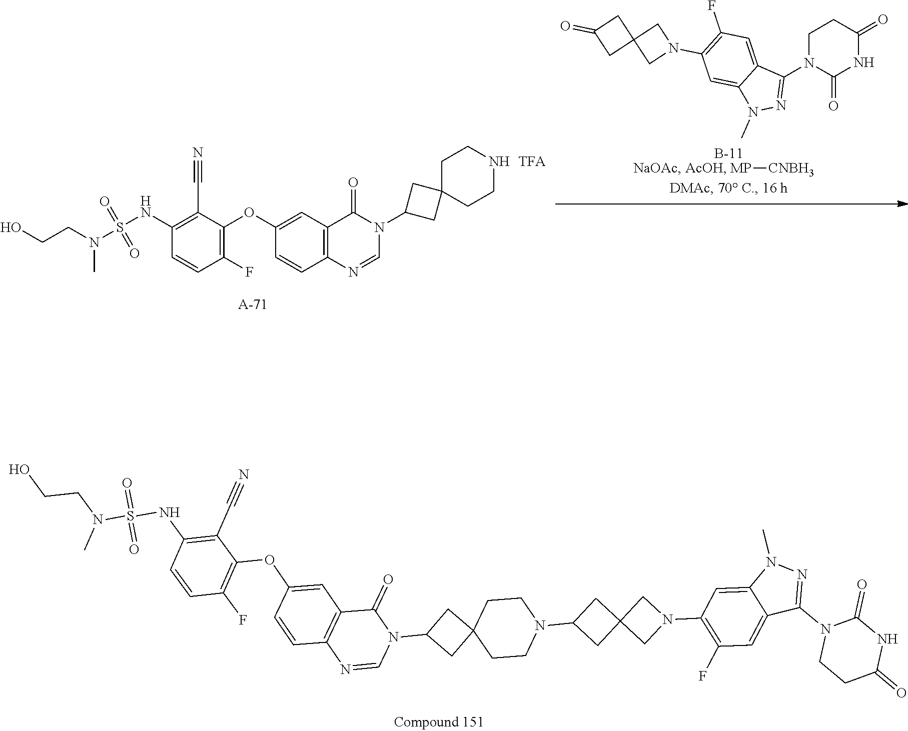

Into a 20 mL pressure vial containing the well-stirred solution of 3-(7-azaspiro[3.5]nonan-2-yl)-6-[2-cyano-6-fluoro-3-[[2-hydroxyethyl(methyl)sulfamoyl]amino]phenoxy]-4-oxo-quinazoline (A-71) (250 mg, 275.86 mol, TFA salt) in DMAc (5 mL) was added NaOAc (67.89 mg, 827.58 mol) at room temperature and stirred for 5 min at the same temperature. To the above mixture, 1-[5-fluoro-1-methyl-6-(6-oxo-2-azaspiro[3.3]heptan-2-yl)indazol-3-yl]hexahydropyrimidine-2,4-dione (B-11) (140.33 mg, 275.86 mol) and AcOH (275.86 mol, 15.78 μL) were added at 0° C. and stirred at room temperature for 3 hours. Subsequently, MP-CNBH₃ (250 mg, 275.86 mol) was added and resulting reaction mixture was stirred at 70° C. for 16 hours. After completion of the reaction, the reaction mixture was filtered through a cotton pad, and the filtrate was concentrated under reduced pressure to get a crude residue which was purified by preparative HPLC [Column: X-SELECT CSH (150 mm*19 mm*5 μm), Mobile phase A: 10 mM NH₄HCO₃ in acetonitrile; Mobile phase B: 100% acetonitrile]. Fractions were lyophilized to afford 6-[2-cyano-6-fluoro-3-[[2-hydroxyethyl(methyl)sulfamoyl]amino]phenoxy]-3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazoline (Compound 151, 65 mg, 68.60 mol, 25% yield) as an off-white solid. UPLC-MS (ES+): m/z 912.7 [M+H]⁺. ¹H NMR (400 MHz, DMSO-d₆): δ 10.51 (s, 1H), 8.38 (s, 1H), 7.76 (d, J=9.20 Hz, 1H), 7.63 (dd, J=3.20, 8.80 Hz, 1H), 7.52-7.40 (m, 1H), 7.35-7.25 (m, 3H), 6.52 (d, J=7.60 Hz, 1H), 4.97-4.90 (m, 1H), 4.02-3.86 (m, 10H), 3.50 (t, J=6.40 Hz, 2H), 3.06 (t, J=6.40 Hz, 2H), 2.90-2.60 (m, 10H), 2.54-2.52 (m, 2H), 2.40-2.25 (m, 6H), 2.00-1.60 (m, 4H).

### Synthesis D81: Synthesis of 6-[2-cyano-3-[[ethyl(methyl)sulfamoyl]amino]-6-fluoro-phenoxy]-3-[2-[1-[4-[4-[(2,6-dioxo-3-piperidyl)amino]-2-fluoro-phenyl]cyclohexyl]-4-piperidyl]ethyl]-4-oxo-quinazoline (Compound 152 and Compound 153)

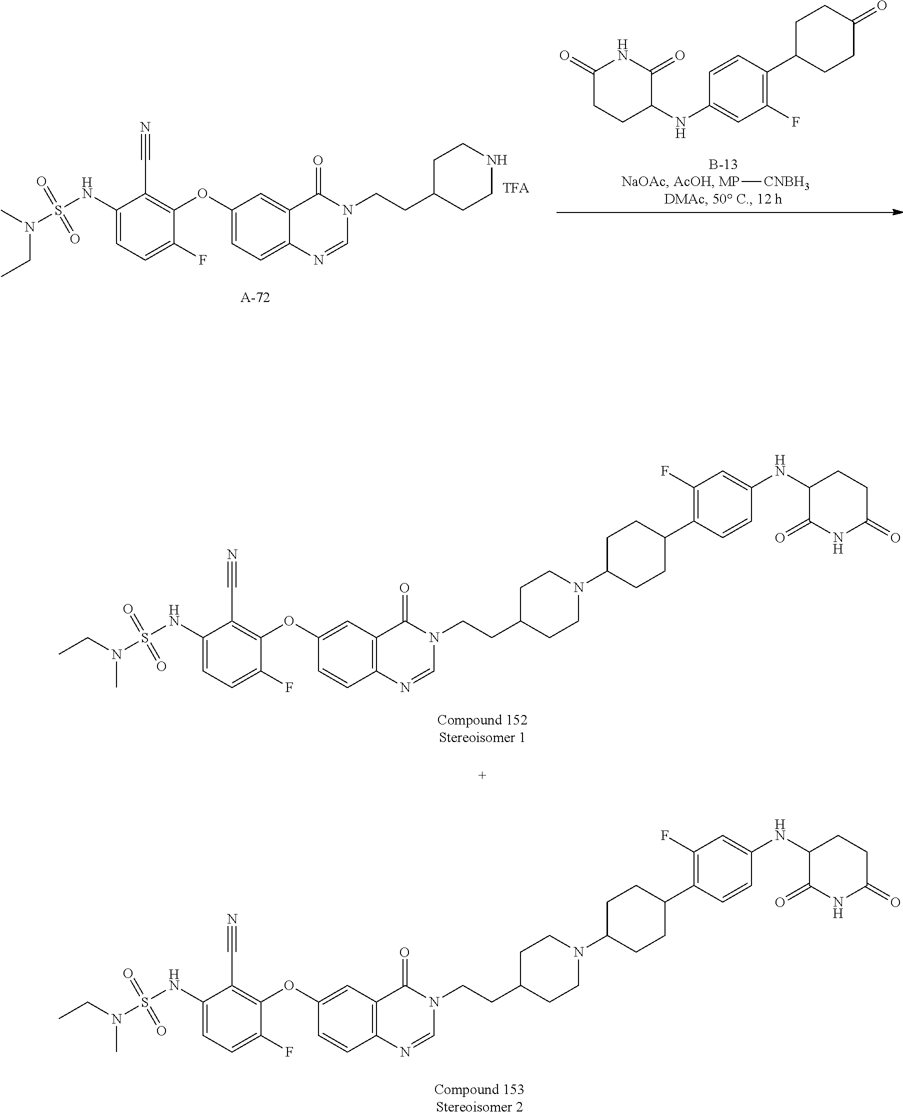

Into a 20 mL pressure vial containing a well stirred solution of 6-[2-cyano-3-[[ethyl(methyl)sulfamoyl]amino]-6-fluoro-phenoxy]-4-oxo-3-[2-(4-piperidyl)ethyl]quinazoline (A-72) (0.2 g, 298.78 mol, TFA salt) in DMAc (5 mL) was added NaOAc (73.53 mg, 896.33 mol) at room temperature and stirred for 15 minutes at the same temperature. After 15 minutes, 3-[3-fluoro-4-(4-oxocyclohexyl)anilino]piperidine-2,6-dione (B-13) (114.94 mg, 358.53 mol) and AcOH (597.55 mol, 58.47 μL) were added at 0° C. and stirring was continued at room temperature for 8 hours. Subsequently, MP-CNBH₃ (200 mg, 298.78 mol) was added and resulting reaction mixture was stirred at 50° C. for 12 hours. After completion of the reaction, the reaction mixture was filtered through cotton pad and filtrate was concentrated under reduced pressure to get a crude residue which was purified by reverse phase column chromatography (Column: Redisep R_{f} Gold® reversed-phase, C18-Teledyne ISCO, 120 g), Mobile phase A: 10 mM NH₄HCO₃ in milli-Q water; Mobile phase B: acetonitrile). Fractions were collected and lyophilized to afford 160 mg of mixture of cis and trans isomers. Further cis and trans isomers were separated by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction was lyophilized to afford Compound 152 (assigned as stereoisomer 1).

Peak-1: 6-[2-cyano-3-[[ethyl(methyl)sulfamoyl]amino]-6-fluoro-phenoxy]-3-[2-[1-[4-[4-[(2,6-dioxo-3-piperidyl)amino]-2-fluoro-phenyl]cyclohexyl]-4-piperidyl]ethyl]-4-oxo-quinazoline (Compound 152, 10.23 mg, 12.06 mol, 4% yield) as an off-white solid. UPLC-MS (ES+): m/z 831.5 [M+H]⁺. ¹H NMR (400 MHz, DMSO-d₆): δ 10.78 (s, 1H), 8.76 (bs, 1H), 8.37 (s, 1H), 7.75 (d, J=8.80 Hz, 1H), 7.62 (dd, J=3.20, 9.00 Hz, 1H), 7.34-7.25 (m, 3H), 6.97 (t, J=8.40 Hz, 1H), 6.50-6.43 (m, 2H), 6.01 (d, J=7.20 Hz, 1H), 4.35-4.25 (m, 1H), 4.05-3.95 (m, 2H), 3.27-3.18 (m, 2H), 3.10-2.90 (m, 4H), 2.70-2.78 (m, 1H), 2.65-2.56 (m, 4H), 2.12-1.91 (m, 5H), 1.90-1.80 (m, 4H), 1.62-1.24 (m, 10H), 1.02 (t, J=7.20 Hz, 3H).

Similarly, the second eluted fraction was lyophilized to afford Compound 153 (assigned as stereoisomer 2).

Peak-2: 6-[2-cyano-3-[[ethyl(methyl)sulfamoyl]amino]-6-fluoro-phenoxy]-3-[2-[1-[4-[4-[(2,6-dioxo-3-piperidyl)amino]-2-fluoro-phenyl]cyclohexyl]-4-piperidyl]ethyl]-4-oxo-quinazoline (Compound 153, 12.19 mg, 14.57 mol, 5% yield) as an off-white solid. UPLC-MS (ES+): m/z 831.5 [M+H]⁺. ¹H NMR (400 MHz, DMSO-d₆): δ 10.79 (s, 1H), 8.60 (bs, 1H), 8.38 (s, 1H), 7.76 (d, J=8.80 Hz, 1H), 7.63 (dd, J=2.8, 8.8 Hz, 1H), 7.33-7.25 (m, 3H), 7.15-7.05 (s, 1H), 6.53-6.44 (m, 2H), 6.04 (s, 1H), 4.35-4.25 (m, 1H), 4.05-3.98 (m, 2H), 3.60-3.50 (m, 1H), 3.25-3.20 (m, 1H), 3.10-2.80 (m, 5H), 2.78-2.70 (m, 1H), 2.60-2.54 (m, 4H), 2.12-2.07 (m, 1H), 2.02-1.60 (m, 14H), 1.47-1.35 (m, 3H), 1.03 (t, J=7.20 Hz, 3H).

### Synthesis D82: Synthesis of 9-[(3R)-3-[6-[2-cyano-3-[[ethyl(methyl)sulfamoyl]amino]-6-fluoro-phenoxy]-4-oxo-quinazolin-3-yl]-1-oxaspiro[4.5]decan-8-yl]-7-[3-(2,4-dioxohexahydropyrimidin-1-yl)-5-fluoro-1-methyl-indazol-6-yl]-3-oxa-9-azabicyclo[3.3.1]nonane (Compound 154 and Compound 155)

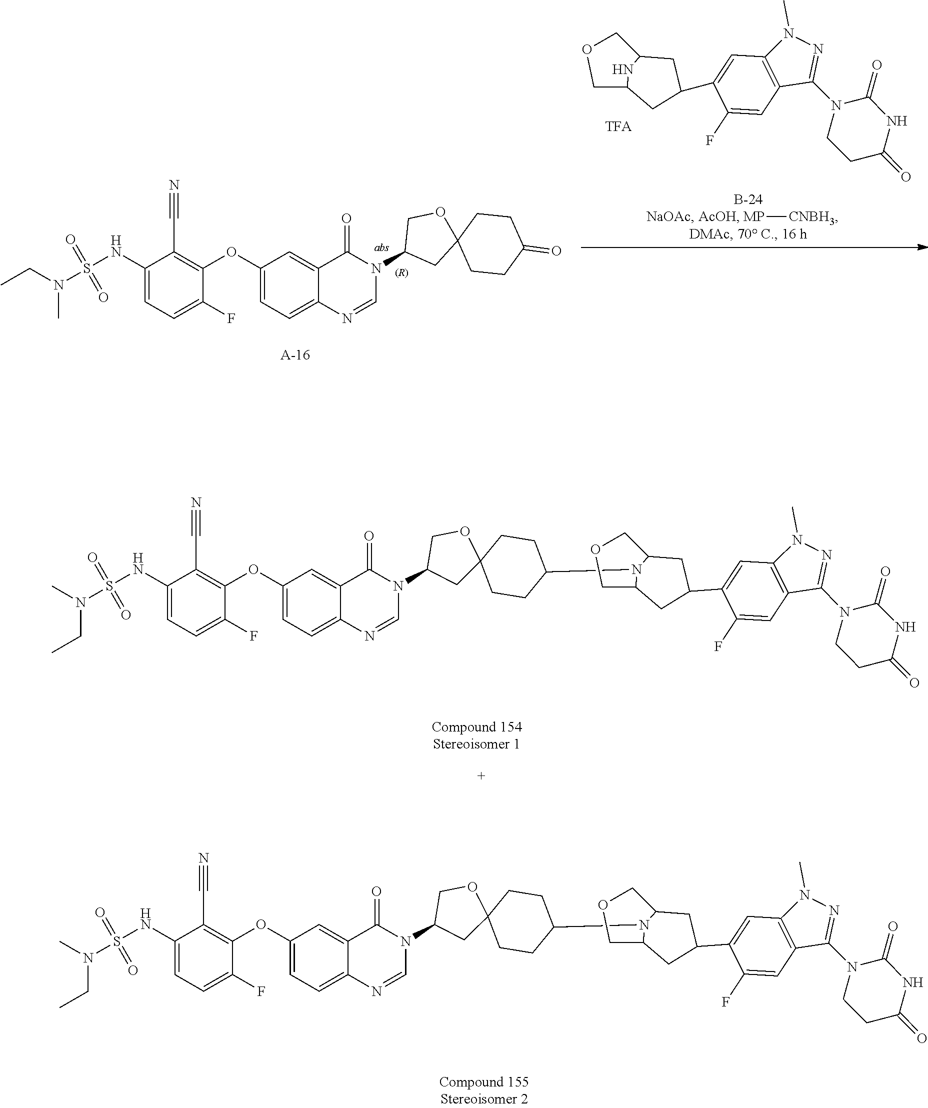

Into a 40 mL pressure vial containing a well-stirred solution of 1-[5-fluoro-1-methyl-6-(3-oxa-9-azabicyclo[3.3.1]nonan-7-yl)indazol-3-yl]hexahydropyrimidine-2,4-dione (B-24) (250 mg, 498.57 μmol, TFA salt) and 6-[2-cyano-3-[[ethyl(methyl) sulfamoyl]amino]-6-fluoro-phenoxy]-4-oxo-3-[(3R)-8-oxo-1-oxaspiro[4.5]decan-3-yl]quinazoline (A-16) (283.99 mg, 498.57 mol) in DMAc (4 mL) were added anhydrous NaOAc (122.70 mg, 1.50 mmol), AcOH (2.49 mmol, 142.57 L) and stirred at room temperature for 1 hour. Finally, MP-CNBH₃ (300 mg, 498.57 mol) was added and stirred at 70° C. for 16 hours. After completion of the reaction, the reaction mixture was diluted with dichloromethane (10 mL), filtered through cotton pad, filtrate was concentrated under reduced pressure to get a crude residue, which was purified by reverse phase by using (RediSep R_{f} Gold® 100 g column) [Mobile phase A: 10 mM NH₄HCO₃ in milli-Q water; Mobile phase B: acetonitrile] while the desire product was eluting at 40-45% of the mobile phase. Fraction was lyophilized to afford 100 mg of mixture of cis and trans isomers.

Chiral SFC separation: Above isomeric mixture was purified by chiral SFC following the below method.

### Method of Analysis for Chiral SFC Separation

Instrument: PIC-22-016
Column: 4EP-(250*30) mm, 5 m
Mobile Phase: CO₂: METHANOL: CAN (1:1) [75:25]
Total Flow: 120 mL/min
Back pressure: 120 bar
Wavelength: 220 nm
Cycle time: 9.0 min

Fast eluting isomer (Peak-1): Fast eluting compound (Peak-1, t_{R}=2.063 min) was collected and concentrated under reduced pressure to afford 9-[(3R)-3-[6-[2-cyano-3-[[ethyl(methyl)sulfamoyl]amino]-6-fluoro-phenoxy]-4-oxo-quinazolin-3-yl]-1-oxaspiro[4.5]decan-8-yl]-7-[3-(2,4-dioxohexahydropyrimidin-1-yl)-5-fluoro-1-methyl-indazol-6-yl]-3-oxa-9-azabicyclo[3.3.1]nonane (Compound 154, 28 mg, 29.68 mol, 6% yield) as an off-white solid. Peak-1 was assigned as stereoisomer 1. UPLC-MS (ES+): m/z 941.7 [M+H]⁺. ¹H NMR (400 MHz, DMSO-d₆): δ 10.55 (s, 1H), 10.16 (bs, 1H), 8.36 (s, 1H), 7.79 (d, J=8.8 Hz, 2H), 7.69 (dd, J=2.80, 9.00 Hz, 1H), 7.58-7.56 (m, 1H), 7.46-7.45 (m, 1H), 7.37-7.35 (m, 2H), 5.29-5.25 (m, 1H), 4.11-4.09 (m, 2H), 4.01 (s, 3H), 3.91 (t, J=6.80 Hz, 2H), 3.70-3.68 (m, 2H), 3.41-3.38 (m, 1H), 3.28-2.95 (m, 6H), 2.77-2.74 (m, 5H), 2.41-2.34 (m, 2H), 2.26-2.15 (m, 2H), 2.04-1.92 (m, 3H), 1.90-1.75 (m, 4H), 1.66-1.62 (m, 1H), 1.49-1.46 (m, 1H), 1.33-1.28 (m, 2H), 1.05 (t, J=7.20 Hz, 3H).

Late eluting isomer (Peak-2): Late eluting compound (Peak-2, t_{R}=2.246 min) was collected and concentrated under reduced pressure to afford 9-[(3R)-3-[6-[2-cyano-3-[[ethyl(methyl)sulfamoyl]amino]-6-fluoro-phenoxy]-4-oxo-quinazolin-3-yl]-1-oxaspiro[4.5]decan-8-yl]-7-[3-(2,4-dioxohexahydropyrimidin-1-yl)-5-fluoro-1-methyl-indazol-6-yl]-3-oxa-9-azabicyclo[3.3.1]nonane (Compound 155, 14 mg, 14.79 mol, 3% yield) as an off-white solid. Peak-2 was assigned as stereoisomer 2. UPLC-MS (ES+): m/z 941.7 [M+H]⁺. ¹H NMR (400 MHz, DMSO-d₆): δ 10.55 (s, 1H), 10.16 (bs, 1H), 8.36 (s, 1H), 7.80-7.78 (m, 2H), 7.69 (dd, J=2.80, 9.00 Hz, 1H), 7.57-7.56 (m, 1H), 7.46-7.44 (m, 1H), 7.39-7.36 (m, 2H), 5.29-5.25 (m, 1H), 4.11-4.08 (m, 2H), 4.02 (s, 3H), 3.91 (t, J=6.40 Hz, 2H), 3.69-3.65 (m, 2H), 3.42-3.40 (m, 1H), 3.32-3.28 (m, 3H), 3.15-3.12 (m, 2H), 2.99-2.97 (m, 1H), 2.77-2.74 (m, 5H), 2.41-2.39 (m, 2H), 2.30-2.22 (m, 2H), 2.05-1.75 (m, 7H), 1.65-1.45 (m, 4H), 1.05 (t, J=7.20 Hz, 3H).

### Synthesis D83: Synthesis of 6-[2-cyano-3-[[ethyl(methyl)sulfamoyl]amino]-6-fluoro-phenoxy]-3-[7-[1-[3-(2,4-dioxohexahydropyrimidin-1-yl)-5-fluoro-1-methyl-indazol-6-yl]-4-piperidyl]-7-azaspiro[3.5]nonan-2-yl]-4-oxo-quinazoline (Compound 156)

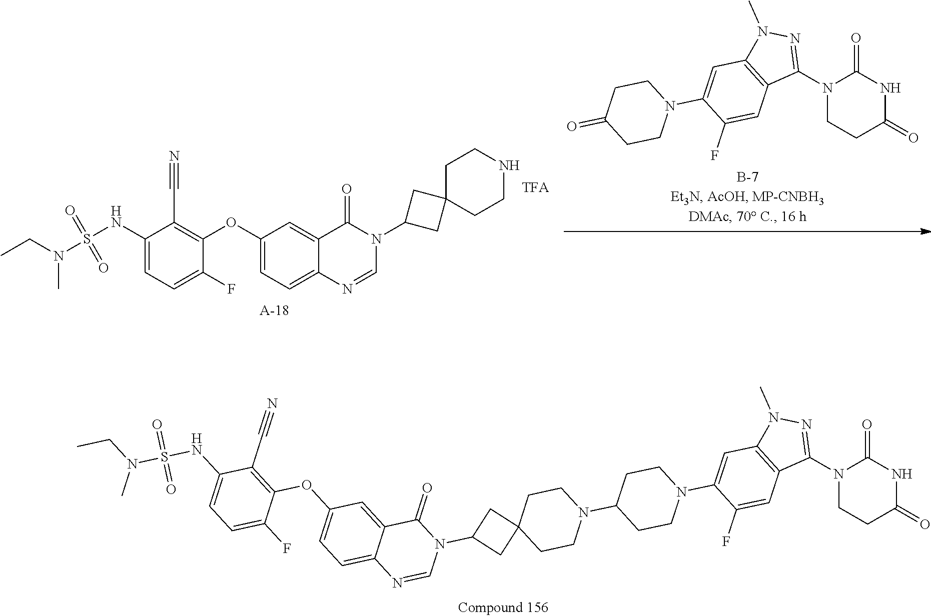

Into a 20 mL pressure vial containing a well-stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (200 mg, 305.52 mol, TFA salt) and 1-[5-fluoro-1-methyl-6-(4-oxo-1-piperidyl)indazol-3-yl]hexahydropyrimidine-2,4-dione (B-7) (109.79 mg, 305.52 mol) in DMAc (3 mL) was added triethylamine (1.53 mmol, 212.91 μL) and mixture was stirred at room temperature for 30 minutes. To the above mixture AcOH (1.53 mmol, 87.36 μL) was added and stirred at room temperature for 1 hour. Finally, MP-CNBH₃ (200 mg, 305.52 mol) was added and resulting reaction mixture was stirred at 70° C. for 16 hours. After completion of the reaction, the reaction mixture was diluted with dichloromethane (10 mL), filtered through cotton pad and filtrate was concentrated under reduced pressure to get a crude residue which was purified by reverse phase by using RediSep R_{f} Gold® 100 g column [Mobile phase A: 10 mM ammonium bicarbonate in milli-Q water; Mobile phase B: acetonitrile] while desired product was eluting at 40-45% of the mobile phase. Fractions were lyophilized to afford crude product (100 mg, LC-MS 99%). ¹H NMR indicated trap of triethyl amine which was further purified by prep HPLC by using following method [Column: X BRIDGE C18 (150 mm*21.0 mm*5 μm) and Mobile phase A: 10 mM ammonium bicarbonate (pH was adjusted to 10 with ammonia) in H₂O, Mobile phase B: 100% acetonitrile, flow: 15 mL/min]. Fraction was lyophilized to get 6-[2-cyano-3-[[ethyl(methyl)sulfamoyl]amino]-6-fluoro-phenoxy]-3-[7-[1-[3-(2,4-dioxohexahydropyrimidin-1-yl)-5-fluoro-1-methyl-indazol-6-yl]-4-piperidyl]-7-azaspiro[3.5]nonan-2-yl]-4-oxo-quinazoline (Compound 156, 42 mg, 47.35 mol, 16% yield) as off-white solid. UPLC-MS (ES+): m/z 884.6 [M+H]⁺. ¹H NMR (400 MHz, DMSO-d₆): δ 10.52 (s, 1H), 9.25 (brs, 1H), 8.39 (s, 1H), 7.76 (d, J=8.80 Hz, 1H), 7.63 (dd, J=2.80, 9.00 Hz, 1H), 7.45-7.29 (m, 4H), 7.14 (d, J=7.20 Hz, 1H), 5.01-4.93 (m, 1H), 3.96 (s, 3H), 3.90 (t, J=6.80 Hz, 2H), 3.59-3.56 (m, 2H), 3.05-2.71 (m, 11H), 2.61 (s, 3H), 2.39-2.33 (m, 3H), 2.09-1.83 (m, 9H), 1.03 (t, J=7.20 Hz, 3H).

### Synthesis D84: Synthesis of (3S)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 157)

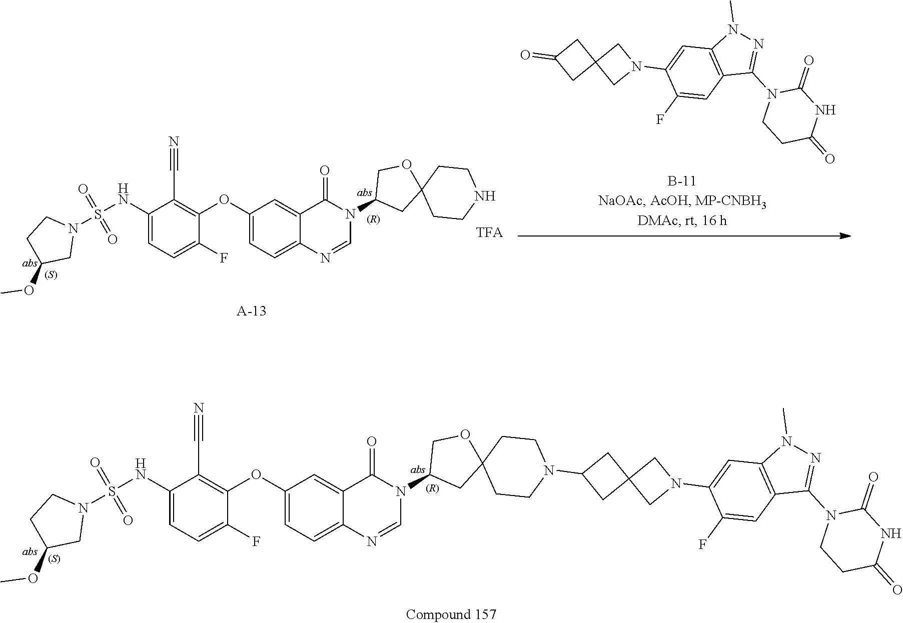

In a 20 mL pressure vial containing the well-stirred solution of (3S)-N-[2-cyano-4-fluoro-3-[3-[(3R)-1-oxa-8-azaspiro[4.5]decan-3-yl]-4-oxo-quinazolin-6-yl]oxy-phenyl]-3-methoxy-pyrrolidine-1-sulfonamide (A-13) (150 mg, 210.48 mol, TFA salt) in DMAc (3.01 mL) was added anhydrous NaOAc (51.80 mg, 631.43 mol) at room temperature and stirred for 30 min at the same temperature. To the above mixture, 1-[5-fluoro-1-methyl-6-(6-oxo-2-azaspiro[3.3]heptan-2-yl)indazol-3-yl]hexahydropyrimidine-2,4-dione (B-11) (83.15 mg, 210.48 mol) and AcOH (210.48 mol, 12.03 μL) were added at 0° C. and resulting reaction mixture was stirred at room temperature for 3 hours. Subsequently, MP-CNBH₃ (200 mg, 210.48 mol) was added, and the reaction mixture was stirred at room temperature for 16 hours. After completion of the reaction, the reaction mixture was filtered through cotton pad and the filtrate was concentrated under reduced pressure to get a crude residue which was purified by reverse phase column chromatography (Column: Redisep R_{f} Gold® reversed-phase C18-Teledyne ISCO, 120 g), [Mobile phase A: 10 mM ammonium bicarbonate in milli-Q water; Mobile phase B: acetonitrile). Fractions were collected and lyophilized to afford 70 mg of a mixture of cis and trans isomers. Further cis and trans isomers were separated by preparative HPLC [Column: X-Bridge C18 (250 mm*19 mm*5 m), Mobile phase A: 10 mm NH₄HCO₃ and NH₄OH (pH-10); Mobile phase B: 100% acetonitrile, Flow rate: 14 mL/min]. Fraction was concentrated under reduced pressure at ° C. and lyophilized to afford (3S)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-methoxy-pyrrolidine-1-sulfonamide (Compound 157, 33.26 mg, 34.61 mol, 16% yield) as an white solid. UPLC-MS (ES+): m/z 954.60 [M+H]⁺. ¹H NMR (400 MHz, DMSO-d₆): δ 10.51 (s, 1H), 9.25 (brs, 1H), 8.32 (s, 1H), 7.77 (d, J=8.80 Hz, 1H), 7.65 (d, J=8.40 Hz, 1H), 7.35-7.25 (m, 4H), 6.52 (d, J=7.20 Hz, 1H), 5.31-5.23 (m, 1H), 4.17-4.08 (m, 2H), 4.06-4.02 (m, 2H), 3.88-3.98 (m, 8H), 3.18 (s, 3H), 3.15-3.06 (m, 2H), 2.73 (t, J=6.80 Hz, 2H), 2.22-2.05 (m, 3H), 2.01-1.90 (m, 2H), 1.88-1.40 (m, 4H).

### Synthesis D85: Synthesis of (3S)-3-[7-[2-cyano-3-[[ethyl(methyl)sulfamoyl]amino]-6-fluoro-phenoxy]quinoxalin-2-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 158)

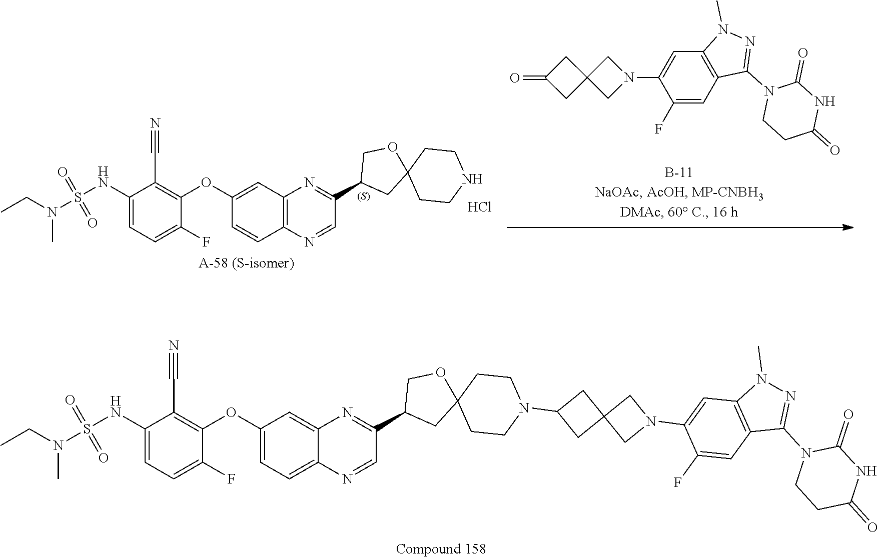

Into a 20 mL glass vial with pressure released cap containing a well-stirred solution of (3S)-3-[7-[2-cyano-3-[[ethyl(methyl)sulfamoyl]amino]-6-fluoro-phenoxy]quinoxalin-2-yl]-1-oxa-8-azaspiro[4.5]decane (A-58) (150 mg, 241.74 mol, HCl salt) in DMAc (3 mL) was added NaOAc (99.15 mg, 1.21 mmol) at room temperature and mixture was stirred for 5 minutes. To the above mixture, AcOH (725.22 mol, 41.47 μL) and 1-[5-fluoro-1-methyl-6-(6-oxo-2-azaspiro[3.3]heptan-2-yl)indazol-3-yl]hexahydropyrimidine-2,4-dione (B-11) (107.73 mg, 290.09 mol) were added at room temperature and stirred for 2 hours at the same temperature. Subsequently, MP-CNBH₃ (150 mg, 241.74 mol) was added and resulting reaction mixture was stirred at 60° C. for 16 hours. After completion, the reaction mixture was filtered through cotton and filtrate was concentrated under reduced pressure to get a crude residue which was purified by reverse phase [Column: Redisep R_{f} Gold®reversed-phase C18-teledyne ISCO, 120 g, Mobile phase A: 10 mM NH₄HCO₃ in milli-Q water; Mobile phase B: acetonitrile). The pure fractions were combined and lyophilized to afford (3S)-3-[7-[2-cyano-3-[[ethyl(methyl)sulfamoyl]amino]-6-fluoro-phenoxy]quinoxalin-2-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 158, 40 mg, 44.53 mol, 18% yield) as an off-white solid. UPLC-MS (ES+): m/z 896.6 [M+H]⁺. ¹H NMR (400 MHz, DMSO-d₆): δ 10.50 (s, 1H), 9.50 (bs, 1H), 8.86 (s, 1H), 8.14 (d, J=9.20 Hz, 1H), 7.71 (dd, J=2.80, 9.20 Hz, 1H), 7.51-7.25 (m, 3H), 7.11 (d, J=2.20 Hz, 1H), 6.53-6.51 (m, 1H), 4.28 (s, 1H), 4.02-3.86 (m, 11H), 3.06-2.75 (m, 5H), 2.73 (t, J=6.80 Hz, 2H), 2.63 (S, 3H), 2.60-2.45 (m, 4H), 2.40-2.21 (m, 4H), 1.91-1.76 (m, 4H), 1.04 (t, J=7.20 Hz, 3H).

### Synthesis D86: Synthesis of (3S)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 159 and Compound 160)

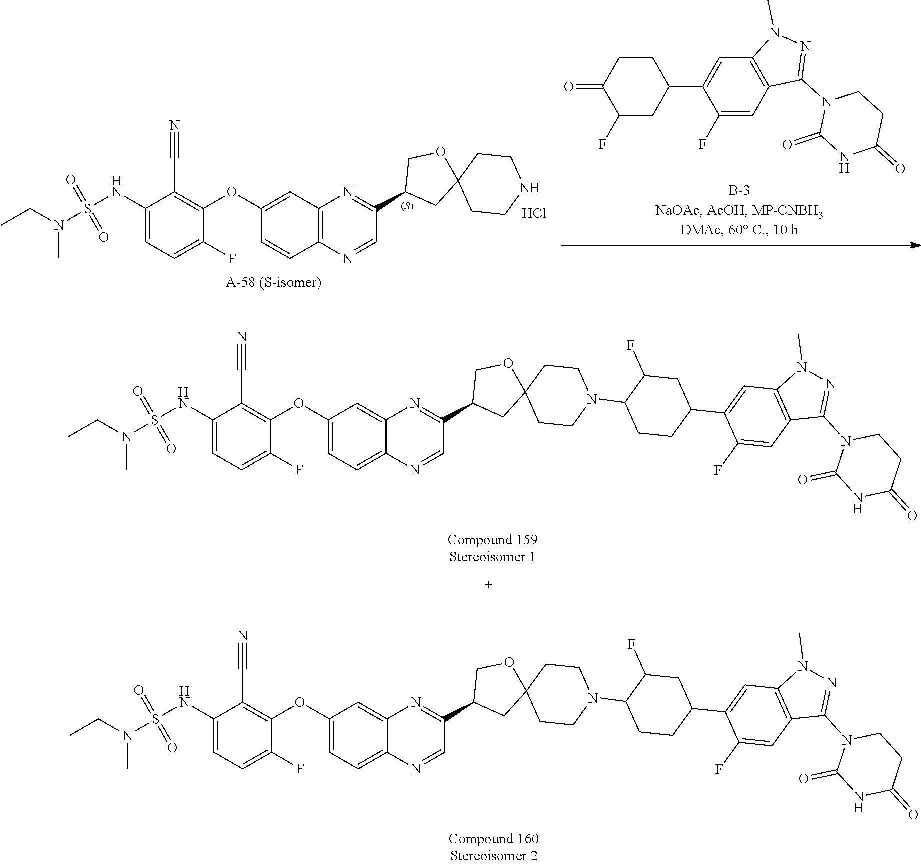

Into a 20 mL glass vial with pressure released cap containing a well stirred solution of (3S)-3-[7-[2-cyano-3-[[ethyl(methyl)sulfamoyl]amino]-6-fluoro-phenoxy]quinoxalin-2-yl]-1-oxa-8-azaspiro[4.5]decane (A-58) (400 mg, 644.64 mol, HCl salt) in DMAc (5 mL) was added NaOAc (264.41 mg, 3.22 mmol) at room temperature and mixture was stirred for 5 minutes. To the above mixture, AcOH (1.93 mmol, 110.6 μL) and 1-[5-fluoro-6-(3-fluoro-4-oxo-cyclohexyl)-1-methyl-indazol-3-yl]hexahydropyrimidine-2,4-dione (B-3) (363.92 mg, 966.95 mol) were added and stirred for 2 hours at the same temperature. Subsequently, MP-CNBH₃ (400 mg, 644.64 mol) was added at room temperature and resulting reaction mixture was stirred at 60° C. for 10 hours. After completion of the reaction, the reaction mixture was filtered through cotton and filtrate was concentrated under reduced pressure to get a crude residue, which was purified by prep HPLC [Column: XBridge C18(150*19*5 um), Mobile phase A: 10 mM ammonium bicarbonate & NH₄OH (pH-10) in mill-Q water; Mobile phase B: acetonitrile]. The fractions were lyophilized to afford first eluted fraction (Peak-1 was assigned as stereoisomer 1) and second eluted fraction (Peak-2 was assigned as stereoisomer 2).

Peak-1: (3S)-3-[7-[2-cyano-3-[[ethyl(methyl)sulfamoyl]amino]-6-fluoro-phenoxy]quinoxalin-2-yl]-8-[(1S,4S)-4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 159, 60 mg, 65.54 mol, 10% yield) as an off-white solid. Peak-1 was assigned as stereoisomer 1. UPLC-MS (ES+): m/z 901.6 [M+H]⁺. H NMR (400 MHz, DMSO-d₆): δ 10.56 (s, 1H), 9.83 (bs, 1H), 8.88 (s, 1H), 8.16 (d, J=9.20 Hz, 1H), 7.72 (dd, J=2.80, 9.20 Hz, 1H), 7.66-7.58 (m, 2H), 7.42-7.39 (m, 2H), 7.17-7.15 (m, 1H), 5.46-5.36 (m, 1H), 4.32-4.23 (m, 1H), 4.05-3.97 (m, 5H), 3.91 (t, J=6.80 Hz, 2H), 3.24-3.21 (m, 2H), 3.14-3.10 (m, 5H), 2.77-2.69 (m, 5H), 2.53-2.51 (m, 1H), 2.39-2.36 (m, 1H), 2.21-2.19 (m, 2H), 2.08-1.76 (m, 9H), 1.05 (t, J=6.80 Hz, 3H).

Peak-2 (3S)-3-[7-[2-cyano-3-[[ethyl(methyl)sulfamoyl]amino]-6-fluoro-phenoxy]quinoxalin-2-yl]-8-[(1R,4S)-4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 160, 37 mg, 40.69 mol, 6% yield) as a white solid. Peak-2 was assigned as stereoisomer 2. UPLC-MS (ES+): m/z 901.5 [M+H]⁺. ¹H NMR (400 MHz, DMSO-d₆): δ 10.56 (s, 1H), 10.03 (bs, 1H), 8.89 (s, 1H), 8.16 (d, J=9.20 Hz, 1H), 7.78-7.76 (m, 1H), 7.73 (dd, J=3.20, 9.00 Hz, 1H), 7.53 (d, J=6.00 Hz, 1H), 7.47 (dd, J=4.00, 9.20 Hz, 1H), 7.39 (d, J=11.20 Hz, 1H), 7.19 (d, J=2.40 Hz, 1H), 5.35-5.25 (m, 1H), 4.25 (s, 1H), 4.03 (s, 3H), 4.00-3.94 (m, 2H), 3.91 (t, J=6.40 Hz, 2H), 3.14 (q, J=7.20 Hz, 2H), 2.77-2.74 (m, 7H), 2.68-2.67 (m, 3H), 2.37-2.35 (m, 2H), 2.14-2.12 (m, 3H), 1.92-1.71 (m, 8H), 1.06 (t, J=7.20 Hz, 3H).

### Synthesis D87: Synthesis of (3R)-3-[7-[2-cyano-3-[[ethyl(methyl)sulfamoyl]amino]-6-fluoro-phenoxy]quinoxalin-2-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 161)

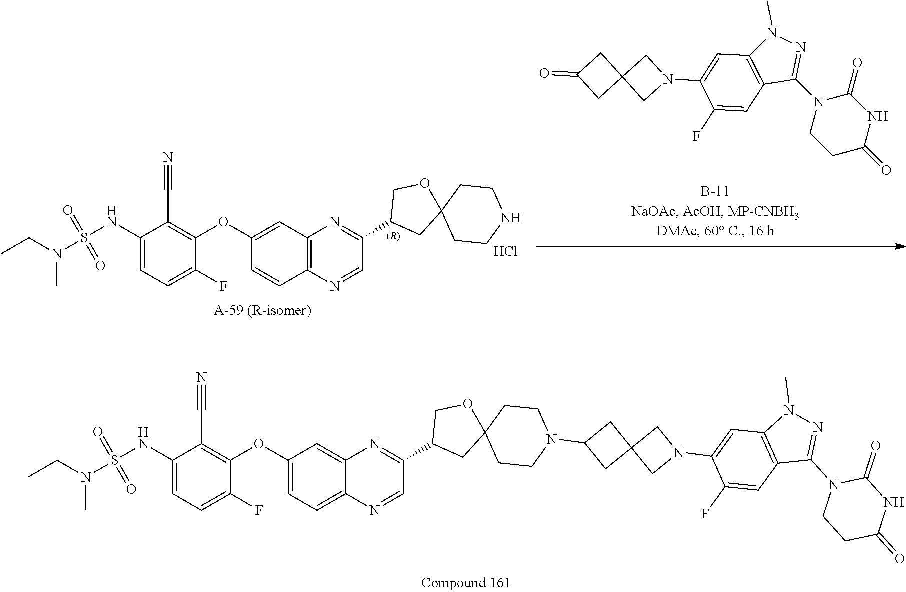

Into a 20 mL glass vial containing a well-stirred solution of (3R)-3-[7-[2-cyano-3-[[ethyl(methyl)sulfamoyl]amino]-6-fluoro-phenoxy]quinoxalin-2-yl]-1-oxa-8-azaspiro[4.5]decane (A-59) (100 mg, 173.29 mol, HCl salt) and 1-[5-fluoro-1-methyl-6-(6-oxo-2-azaspiro[3.3]heptan-2-yl)indazol-3-yl]hexahydropyrimidine-2,4-dione (B-11) (77.22 mg, 207.95 mol) in DMAc (1.0 mL) was added NaOAc (71.07 mg, 866.45 mol) and the resulting mixture was stirred for 1 h at room temperature. To the above mixture, AcOH (519.87 mol, 30.02 L) was added and stirred at room temperature for 2 hours. Subsequently, MP-CNBH₃ (150 mg, 173.29 mol) was added, and reaction mixture was stirred at 60° C. for 16 hours. After completion the reaction, reaction mixture was filtered through sintered funnel and the filtrate was concentrated under reduced pressure to get a crude residue which was purified by reverse phase [Column: R_{f} Gold®reversed-phase C18-teledyne ISCO, 120 g), Mobile phase A: 10 mM NH₄HCO₃ in milliQ-water; Mobile phase B: acetonitrile]. Obtained crude product was further purified by prep HPLC [Column: X Bridge C18(150*19*5 um), Mobile phase A: 10 mM NH₄HCO₃ & NH₄OH (pH-10) in milli-Q water; Mobile phase B: acetonitrile]. The fraction was lyophilized to afford (3R)-3-[7-[2-cyano-3-[[ethyl(methyl)sulfamoyl]amino]-6-fluoro-phenoxy]quinoxalin-2-yl]-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decane (Compound 161, 25 mg, 27.66 mol, 16% yield) as an white solid. UPLC-MS (ES+): m/z 896.2 [M+H]⁺. ¹H NMR (400 MHz, DMSO-d₆): δ 10.51 (s, 1H), 9.60 (bs, 1H), 8.87 (s, 1H), 8.15 (d, J=9.20 Hz, 1H), 7.72-7.68 (m, 1H), 7.54 (bs, 1H), 7.37-7.34 (m, 1H), 7.27 (d, J=12.80 Hz, 1H), 7.16-7.12 (m, 1H), 6.53-6.51 (m, 1H), 4.30-4.24 (m, 1H), 4.03-3.86 (m, 10H), 3.11-2.98 (m, 4H), 2.73 (t, J=6.80 Hz, 2H), 2.68-2.64 (m, 3H), 2.51-2.45 (m, 5H), 2.34-2.33 (m, 1H), 2.28-2.19 (m, 3H), 1.92-1.76 (m, 5H), 1.04 (t, J=7.20 Hz, 3H).

### Synthesis D88: Synthesis of (3R)-3-[7-[2-cyano-3-[[ethyl(methyl)sulfamoyl]amino]-6-fluoro-phenoxy]quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 162 and Compound 163)

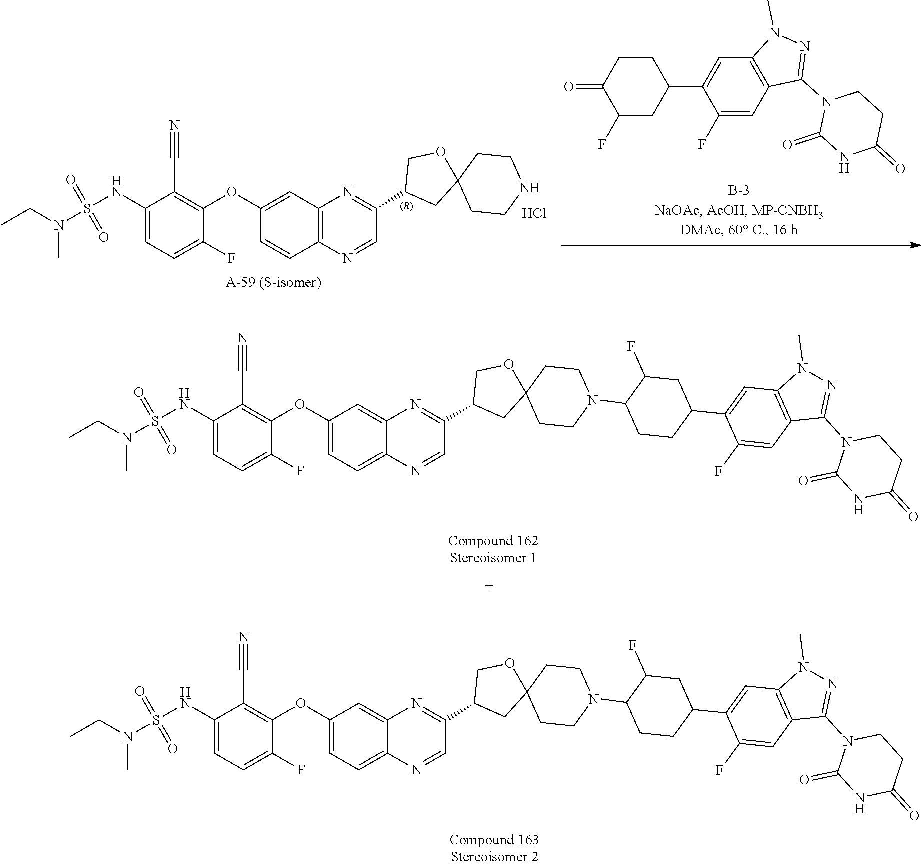

Into a 20 mL glass vial containing a well-stirred solution of (3R)-3-[7-[2-cyano-3-[[ethyl(methyl)sulfamoyl]amino]-6-fluoro-phenoxy]quinoxalin-2-yl]-1-oxa-8-azaspiro[4.5]decane (A-59) (300 mg, 519.87 mol, HCl salt) and 1-[5-fluoro-6-(3-fluoro-4-oxo-cyclohexyl)-1-methyl-indazol-3-yl]hexahydropyrimidine-2,4-dione (B-3) (293.48 mg, 779.80 mol) in DMAc (5.0 mL) was added NaOAc anhydrous (213.22 mg, 2.60 mmol) and the resulting mixture was stirred for 1 h at room temperature. To the above mixture, AcOH (1.56 mmol, 90.05 μL) was added and stirred at room temperature for 2 hours. Subsequently, MP-CNBH₃ (450 mg, 519.87 mol) was added and resulting mixture was stirred at 60° C. for 16 hours. After completion of the reaction, the reaction mixture was filtered through sintered funnel and the filtrate was concentrated under reduced pressure to get a crude residue which was purified by preparative HPLC [Column: XBridge C18 (150 mm*19 mm*5 μm), Mobile phase A: 10 mm NH₄HCO₃ & NH₄OH (pH-10) in milli-Q water; Mobile phase B: acetonitrile]. Fractions were lyophilized to afford the first eluted fraction as a Peak-1 (Compound 162, assigned as stereoisomer 1) and similarly the second eluted fraction as a Peak-2 (Compound 163, assigned as stereoisomer 2).

Peak-1: (3R)-3-[7-[2-cyano-3-[[ethyl(methyl)sulfamoyl]amino]-6-fluoro-phenoxy]quinoxalin-2-yl]-8-[(1S,4S)-4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 162, 33 mg, 35.59 mol, 7% yield) as an off-white solid. Peak-1 was assigned as stereoisomer 1. UPLC-MS (ES+): m/z 901.6 [M+H]⁺. ¹H NMR (400 MHz, DMSO-d₆): δ 10.56 (s, 1H), 9.75 (bs, 1H), 8.88 (s, 1H), 8.15 (d, J=9.20 Hz, 1H), 7.71 (dd, J=2.80, 9.20 Hz, 1H), 7.66-7.58 (m, 2H), 7.41-7.38 (m, 2H), 7.15 (d, J=2.40 Hz, 1H), 5.45-5.33 (m, 1H), 4.28 (s, 1H), 4.02-3.98 (m, 5H), 3.91 (t, J=6.40 Hz, 2H), 3.32-3.21 (m, 1H), 3.10-3.05 (m, 6H), 2.75 (t, J=6.80 Hz, 2H), 2.72-2.62 (m, 4H), 2.35-2.33 (m, 1H), 2.20-2.19 (m, 2H), 2.00-1.75 (m, 9H), 1.05 (t, J=7.20 Hz, 3H).

Peak-2: (3R)-3-[7-[2-cyano-3-[[ethyl(methyl)sulfamoyl]amino]-6-fluoro-phenoxy]quinoxalin-2-yl]-8-[(1S,4R)-4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 163.31 mg, 33.76 mol, 6% yield) as an off-white solid. Peak-2 was assigned as stereoisomer 2. UPLC-MS (ES+): m/z 901.6 [M+H]⁺. ¹H NMR (400 MHz, DMSO-d₆): δ 10.56 (s, 1H), 10.07 (bs, 1H), 8.89 (s, 1H), 8.16 (d, J=9.20 Hz, 1H), 7.80-7.70 (m, 2H), 7.53 (d, J=6.00 Hz, 1H), 7.47-7.45 (m, 1H), 7.40 (d, J=11.20 Hz, 1H), 7.19 (d, J=2.4 Hz, 1H), 5.30-5.20 (m, 1H), 4.28-4.22 (m, 1H), 4.03 (s, 3H), 4.00-4.89 (m, 4H), 3.16-3.11 (m, 2H), 2.77-2.74 (m, 7H), 2.68-2.60 (m, 2H), 2.37-2.32 (m, 1H), 2.29-1.70 (m, 12H), 1.05 (t, J=7.20 Hz, 3H).

### Synthesis D89: Synthesis of 3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-6-[3-[[ethyl(methyl)sulfamoyl]amino]-2,6-difluoro-phenoxy]-4-oxo-quinazoline (Compound 164)

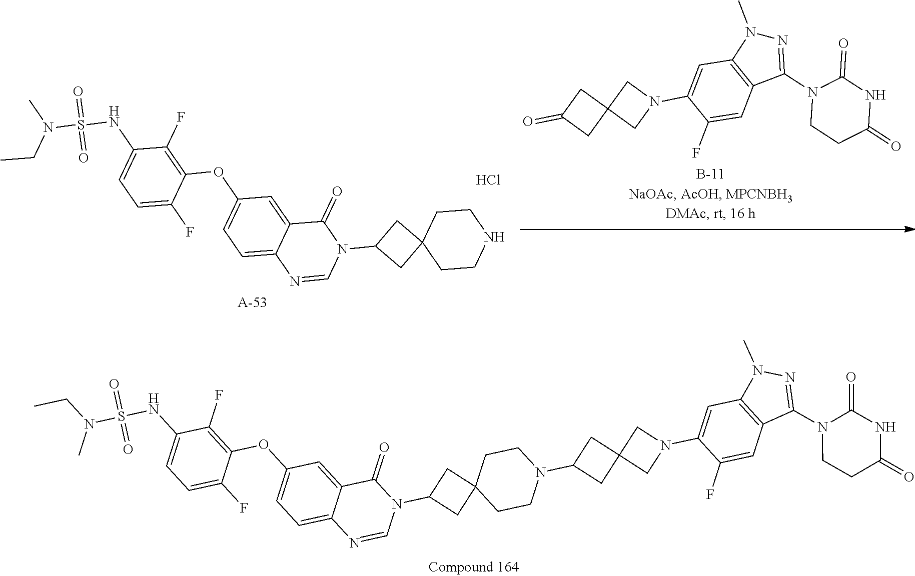

Into a 20 mL glass vial with pressure released cap containing a well-stirred solution of 3-(7-azaspiro[3.5]nonan-2-yl)-6-[3-[[ethyl(methyl)sulfamoyl]amino]-2,6-difluoro-phenoxy]-4-oxo-quinazoline (A-53) (110 mg, 160.16 mol, HCl salt) and 1-[5-fluoro-1-methyl-6-(6-oxo-2-azaspiro[3.3]heptan-2-yl)indazol-3-yl]hexahydropyrimidine-2,4-dione (B-11) (68.37 mg, 176.18 mol) in anhydrous DMAc (2 mL) were added anhydrous NaOAc (26.28 mg, 320.32 mol) followed by AcOH (160.16 mol, 9.16 μL) and stirred at room temperature for 10 min. Subsequently, MP-CNBH₃ (110 mg, 160.16 mol) was added, and reaction mixture was stirred at room temperature for 16 hours. After completion of the reaction, the reaction mixture was filtered and filtrate was concentrated under reduced pressure to get a crude residue which was purified by preparative HPLC [Column: X-Bridge C18 (150 mm*19 mm*5 μm), Mobile phase A: 10 mM NH₄HCO₃ & NH₄OH_pH 10 in milli-Q water; Mobile phase B: acetonitrile]. The fraction was lyophilized to afford 3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-6-[3-[[ethyl(methyl)sulfamoyl]amino]-2,6-difluoro-phenoxy]-4-oxo-quinazoline (Compound 164, 22 mg, 24.34 mol, 15% yield) as an off-white solid. UPLC-MS (ES+): m/z 889.4 [M+H]⁺. ¹HNMR (400 MHz, DMSO-d₆): δ 10.50 (s, 1H), 9.76 (bs, 1H), 8.38 (s, 1H), 7.77 (d, J=8.80 Hz, 1H), 7.66 (dd, J=2.80, 8.80 Hz, 1H), 7.45-7.34 (m, 2H), 7.31 (d, J=2.80 Hz, 1H), 7.25 (d, J=12.80 Hz, 1H), 6.49 (d, J=7.60 Hz, 1H), 4.92-4.85 (m, 1H), 3.99 (s, 2H), 3.89-3.86 (m, 7H), 3.09 (q, J=7.20 Hz, 2H), 2.74-2.71 (m, 5H), 2.60-2.55 (m, 1H), 2.30-2.08 (m, 9H), 2.05-1.95 (m, 2H), 1.75-1.53 (m, 4H), 1.00 (t, J=7.20 Hz, 3H).

### Synthesis D90: Synthesis of (3R)-3-[6-[2-cyano-6-fluoro-3-[[2-hydroxyethyl(methyl)sulfamoyl]amino]phenoxy]-4-oxo-quinazolin-3-yl]-8-[3-[3-(2,4-dioxohexahydropyrimidin-1-yl)-5-fluoro-1-methyl-indazol-6-yl]cyclobutyl]-1-oxa-8-azaspiro[4.5]decane (Compound 165)

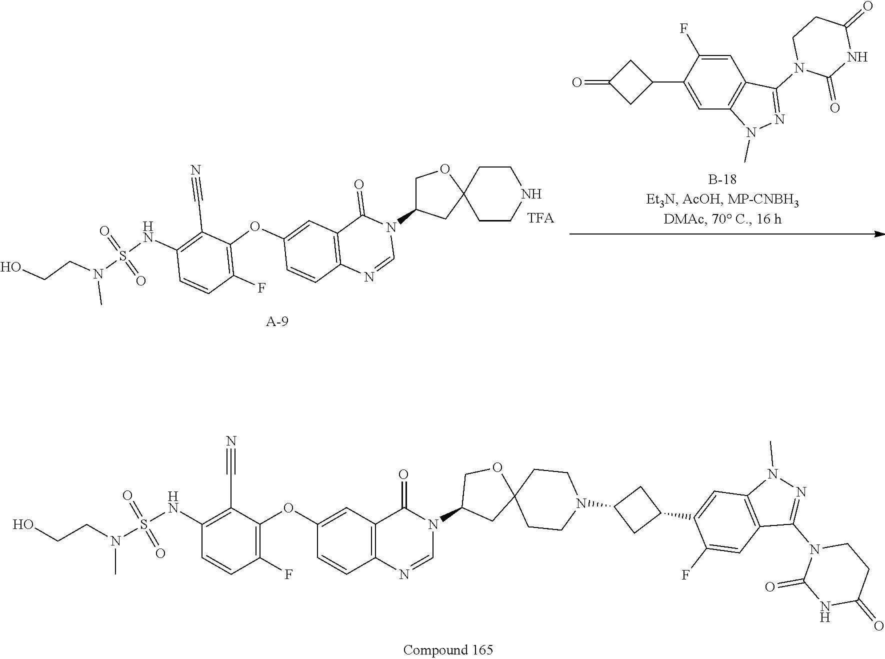

Into a 10 mL pressure vial containing a well-stirred solution of (3R)-3-[6-[2-cyano-6-fluoro-3-[[2-hydroxyethyl(methyl)sulfamoyl]amino]phenoxy]-4-oxo-quinazolin-3-yl]-1-oxa-8-azaspiro[4.5]decane (A-9) (90 mg, 110.10 μmol, TFA salt) in DMAc (1.5 mL) was added triethylamine (550.51 μmol, 76.73 μL) at room temperature and stirred for 1 h under nitrogen atmosphere. To the above mixture, 1-[5-fluoro-1-methyl-6-(3-oxocyclobutyl)indazol-3-yl]hexahydropyrimidine-2,4-dione (B-18) (64.94 mg, 165.15 μmol) and AcOH (661.17 μg, 11.01 μmol) were added and stirred for 5 h at room temperature. Subsequently, MP-CNBH₃ (100 mg, 110.10 μmol) was added, and reaction mixture was stirred at 70° C. for 16 hours. After completion of the reaction, the reaction mixture was filtered and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water; Mobile phase B: acetonitrile]. The fractions were lyophilized to afford (3R)-3-[6-[2-cyano-6-fluoro-3-[[2-hydroxyethyl(methyl)sulfamoyl]amino]phenoxy]-4-oxo-quinazolin-3-yl]-8-[3-[3-(2,4-dioxohexahydropyrimidin-1-yl)-5-fluoro-1-methyl-indazol-6-yl]cyclobutyl]-1-oxa-8-azaspiro[4.5]decane (Compound 165, 9 mg, 9.79 μmol, 9% yield) as an off-white solid. UPLC-MS (ES+): m/z 887.3 [M+H]⁺. ¹H NMR (400 MHz, DMSO-d₆): δ 10.56 (s, 1H), 8.32 (s, 1H), 7.77 (d, J=8.80 Hz, 1H), 7.65 (dd, J=2.80, 8.80 Hz, 1H), 7.55-7.42 (m, 2H), 7.42-7.29 (m, 3H), 5.39-5.19 (m, 1H), 4.21-4.09 (m, 2H), 4.02 (s, 3H), 3.91 (t, J=6.80 Hz, 2H), 3.50 (t, J=6.40 Hz, 2H), 3.07 (t, J=6.00 Hz, 2H), 2.77 (t, J=6.80 Hz, 3H), 2.72-2.69 (m, 3H), 2.66-2.62 (m, 1H), 2.46-2.38 (m, 1H), 2.31-2.18 (m, 2H), 2.17-2.11 (m, 1H), 1.99-1.81 (m, 3H), 1.80-1.70 (m, 1H).

### Synthesis D91: Synthesis of 3-[7-[4-[4-[(2,6-Dioxo-3-piperidyl)amino]-2-fluoro-phenyl]cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 166 and Compound 167)

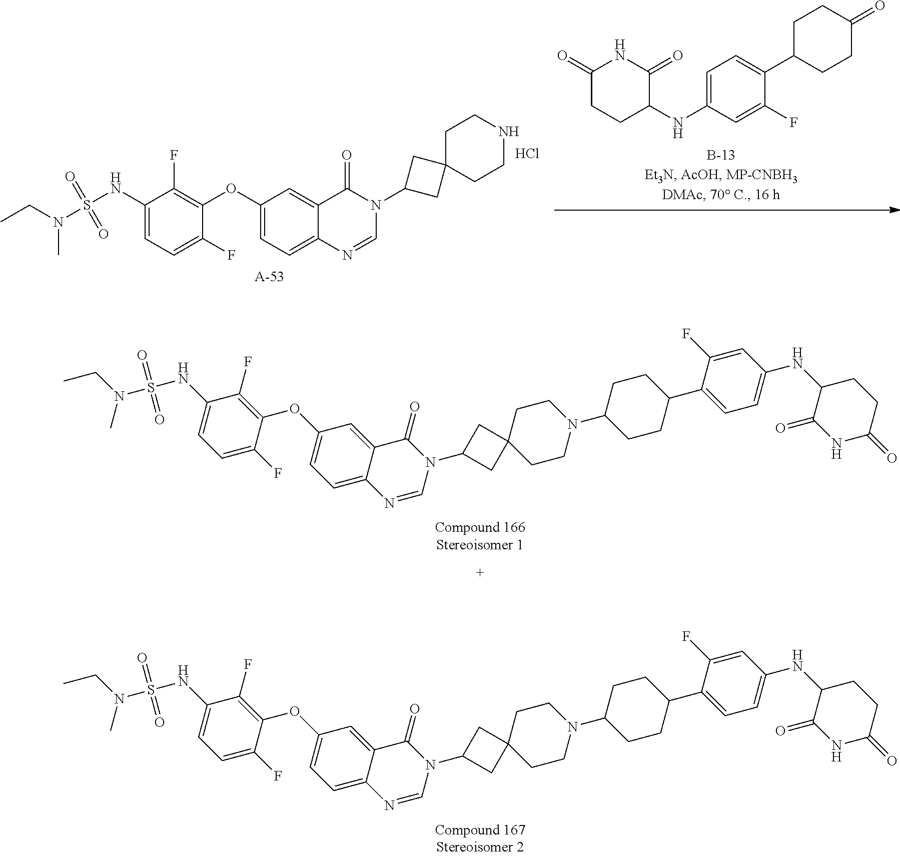

Into a 20 mL pressure vial containing a well-stirred solution of 3-(7-azaspiro[3.5]nonan-2-yl)-6-[3-[[ethyl(methyl)sulfamoyl]amino]-2,6-difluoro-phenoxy]-4-oxo-quinazoline (A-53) (250 mg, 280.68 mol, HCl salt) in DMAc (3.5 mL) was added triethylamine (1.40 mmol, 195.60 μL) at room temperature under nitrogen atmosphere and mixture was stirred for 1 hour. Subsequently, 3-[3-fluoro-4-(4-oxocyclohexyl)anilino]piperidine-2,6-dione (B-13) (134.03 mg, 421.02 μmol) and AcOH (1.403 mmol, 80 μL) were added and resulting mixture was stirred for 4 hours at room temperature. Finally, MP-CNBH₃ (270 mg, 280.68 mol) was added, and the reaction mixture was stirred at 70° C. for 16 hours. After completion of the reaction, the reaction mixture was filtered and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water; Mobile phase B: acetonitrile]. The first eluting fraction (Peak-1) was lyophilized to afford Compound 166 (assigned as stereoisomer 1).

Peak-1: 3-[7-[4-[4-[(2,6-dioxo-3-piperidyl)amino]-2-fluoro-phenyl]cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 166, 9 mg, 10.54 mol, 4% yield) as an off-white solid. UPLC-MS (ES+): m/z 836.3 [M+H]⁺. ¹H NMR (400 MHz, DMSO-d₆): δ 10.70 (m, 1H), 8.37 (s, 1H), 7.76 (d, J=8.80 Hz, 1H), 7.64 (dd, J=2.80, 9.00 Hz, 1H), 7.49-7.21 (m, 3H), 6.98 (t, J=8.80 Hz, 1H), 6.58-6.31 (m, 2H), 5.94 (d, J=7.60 Hz, 1H), 4.99-4.79 (m, 1H), 4.32-4.18 (m, 1H), 3.06 (q, J=6.80 Hz, 2H), 2.80-2.72 (m, 2H), 2.46-2.13 (m, 9H), 2.12-2.06 (m, 1H), 2.02-1.98 (m, 2H), 1.96-1.53 (m, 7H), 1.52-1.35 (m, 4H), 1.01 (t, J=7.20 Hz, 3H).

Similarly, the second eluted fraction (Peak-2) was lyophilized to afford Compound 167 (assigned as stereoisomer 2).

Peak-2: 3-[7-[4-[4-[(2,6-dioxo-3-piperidyl)amino]-2-fluoro-phenyl]cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 167, 31 mg, 35.81 mol, 13% yield) as an off-white solid. UPLC-MS (ES+): m/z 836.3 [M+H]⁺. ¹H NMR (400 MHz, DMSO-d₆): δ 10.78 (m, 1H), 8.38 (s, 1H), 7.76 (d, J=8.80 Hz, 1H), 7.64 (dd, J=2.80, 9.00 Hz, 1H), 7.49-7.29 (m, 3H), 6.98 (t, J=8.80 Hz, 1H), 6.51-6.39 (m, 2H), 5.94 (d, J=7.60 Hz, 1H), 4.99-4.80 (m, 1H), 4.38-4.21 (m, 1H), 3.20-3.18 (m, 1H), 3.08 (q, J=7.20 Hz, 2H), 2.82-2.68 (m, 6H), 2.39-2.30 (m, 3H), 2.28-2.19 (m, 3H), 2.17-2.03 (m, 1H), 2.02-1.93 (m, 2H), 1.92-1.62 (m, 8H), 1.52-1.42 (m, 4H), 1.00 (t, J=7.20 Hz, 3H).

### Synthesis D92: Synthesis of (3R)-3-[6-[2-cyano-6-fluoro-3-[[2-hydroxyethyl(methyl)sulfamoyl]amino]phenoxy]-4-oxo-quinazolin-3-yl]-8-[3-[4-[(2,6-dioxo-3-piperidyl)amino]-2-fluoro-phenyl]cyclobutyl]-1-oxa-8-azaspiro[4.5]decane (Compound 168)

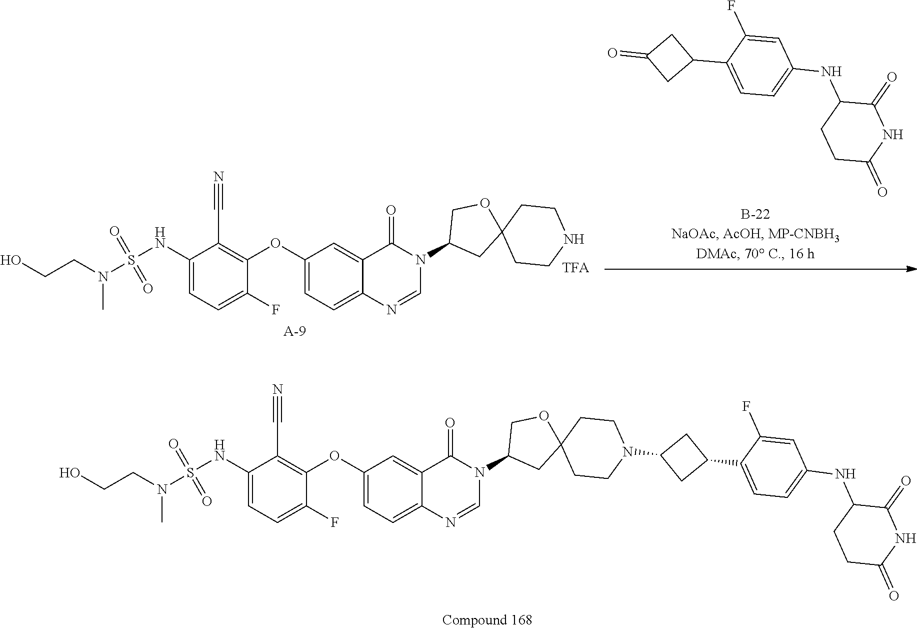

Into a 40 mL pressure vial containing a well-stirred solution of (3R)-3-[6-[2-cyano-6-fluoro-3-[[2-hydroxyethyl(methyl)sulfamoyl]amino]phenoxy]-4-oxo-quinazolin-3-yl]-1-oxa-8-azaspiro[4.5]decane (A-9) (150.00 mg, 220.05 mol, TFA salt) in anhydrous DMAc (3 mL) were added NaOAc (90.26 mg, 1.10 mmol) and AcOH (660.14 mol, 37.79 μL). The resulting reaction mixture was stirred at ambient temperature for 10 min, 3-[3-fluoro-4-(3-oxocyclobutyl)anilino]piperidine-2,6-dione (B-22) (121.59 mg, 330.07 mol) was added at ambient temperature. The resulting mixture was stirred at room temperature for 1 hour. Finally, MP-CNBH₃ (10 mg, 220.05 mol) was added and the reaction mixture was stirred at 70° C. for 16 hours. After the completion of the reaction, the reaction mixture was filtered and filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fraction was lyophilized to afford impure product 100 mg and again re-purified by preparative HPLC [Column: X-Bridge C18 (250 mm*19 mm*5 m), Mobile phase A: 10 mM NH₄HCO₃ in milli-Q water; Mobile phase B: acetonitrile]. The fraction was lyophilized to afford (3R)-3-[6-[2-cyano-6-fluoro-3-[[2-hydroxyethyl(methyl)sulfamoyl]amino]phenoxy]-4-oxo-quinazolin-3-yl]-8-[3-[4-[(2,6-dioxo-3-piperidyl)amino]-2-fluoro-phenyl]cyclobutyl]-1-oxa-8-azaspiro[4.5]decane (Compound 168, 35 mg, 41.25 mol, 19% yield) as an off-white solid. UPLC-MS (ES+): m/z 847.3 [M+H]⁺. ¹H NMR (400 MHz, DMSO-d₆): δ 10.79 (s, 1H), 9.38 (bs, 1H), 8.31 (s, 1H), 7.76 (d, J=8.80 Hz, 1H), 7.64 (dd, J=2.80, 8.80 Hz, 1H), 7.48-7.33 (m, 1H), 7.36-7.30 (m, 2H), 7.04 (t, J=8.80 Hz, 1H), 6.57-6.30 (m, 2H), 6.07 (d, J=8.00 Hz, 1H), 5.30-5.27 (m, 1H), 4.35-4.32 (m, 1H), 4.18-4.11 (m, 2H), 3.50 (t, J=6.40 Hz, 3H), 3.21-3.17 (m, 1H), 3.05 (t, J=6.40 Hz, 3H), 2.95-2.80 (m, 2H), 2.74-2.68 (m, 5H), 2.60-2.51 (m, 3H), 2.45-2.30 (m, 1H), 2.15-2.06 (m, 5H), 1.92-1.85 (m, 5H).

### Synthesis D93: Synthesis of (3S)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 169 and Compound 170)

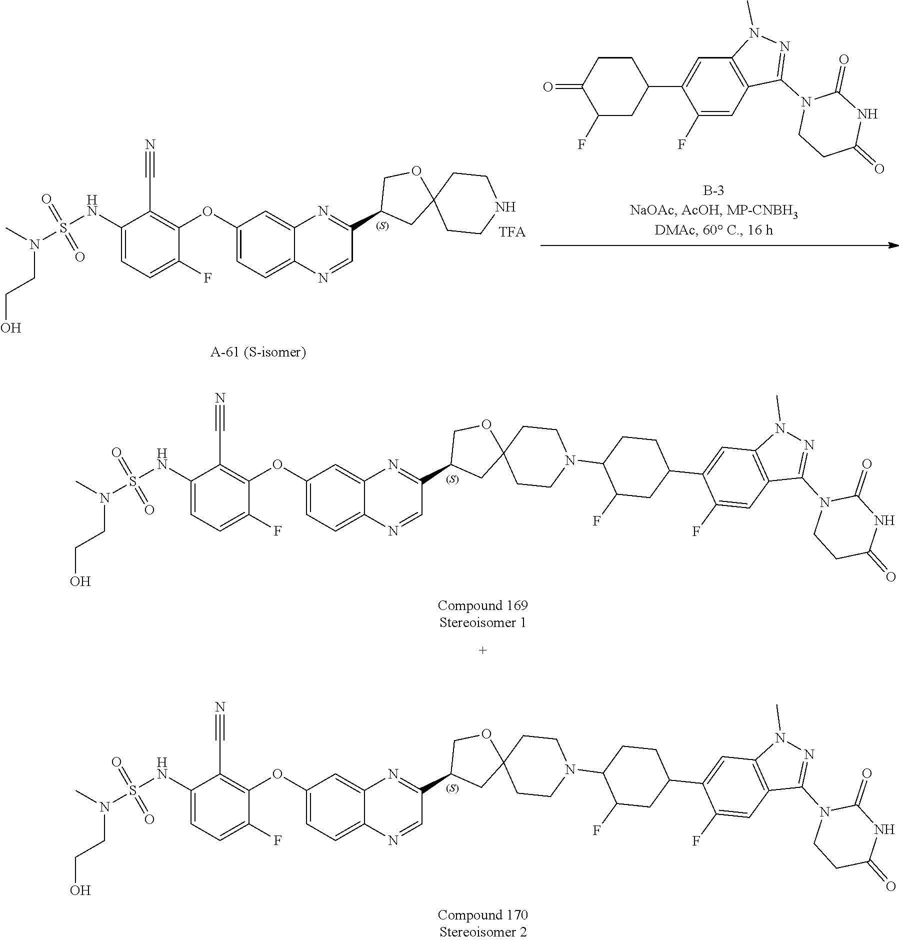

Into a 20 mL glass vial with pressure released cap containing a well-stirred solution of (3S)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-61) (300 mg, 281.82 mol, TFA salt) in DMAc (5 mL) was added NaOAc (115.59 mg, 1.41 mmol) at room temperature and reaction mixture was stirred at the same temperature for 5 minutes. To the above mixture, AcOH (845.47 mol, 47.90 μL) and 1-[5-fluoro-6-(3-fluoro-4-oxo-cyclohexyl)-1-methyl-indazol-3-yl]hexahydropyrimidine-2,4-dione (B-3) (159.10 mg, 422.74 mol) were added at room temperature. The reaction mixture was stirred for 2 hours at the same temperature. Subsequently, MP-CNBH₃ (300 mg, 281.82 mol) was added at room temperature. Resulting reaction mixture was stirred at 60° C. for 16 hours. After completion of the reaction, reaction mixture was filtered through cotton and filtrate was concentrated under reduced pressure to get a crude residue which was purified by prep HPLC [Column: X Bridge C18 (150 mm*19 mm*5 μm), Mobile phase A: 10 mm NH₄HCO₃ & NH₄OH (pH-10) in milli-Q water; Mobile phase B: acetonitrile]. The fractions were lyophilized to afford the first eluted fraction as Peak-1 (Compound 169, assigned as stereoisomer 1) and similarly the second eluted fraction as Peak-2 (Compound 170, assigned as stereoisomer 2). Peak-1: (3S)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 169.47 mg, 51.05 mol, 18% yield) as an off-white solid. Peak-1 was assigned as stereoisomer 1. UPLC-MS (ES+): m/z 917.3 [M+H]⁺. ¹H NMR (400 MHz, DMSO-d₆): δ 10.53 (s, 1H), 9.76 (bs, 1H), 8.87 (s, 1H), 8.14 (d, J=9.20 Hz, 1H), 7.70 (dd, J=2.80, 9.20 Hz, 1H), 7.60-7.38 (m, 4H), 7.17 (s, 1H), 5.41-5.28 (m, 1H), 4.32-4.22 (m, 1H), 4.00-3.89 (m, 8H), 3.51 (t, J=6.00 Hz, 2H), 3.30-3.21 (m, 1H), 3.11-2.95 (m, 6H), 2.77-2.68 (m, 5H), 2.45-2.35 (m, 1H), 2.33-0.00 (m, 2H), 2.06-1.75 (m, 10H).

Peak-2: (3S)-3-[7-[2-cyano-6-fluoro-3-[[2-hydroxyethyl(methyl)sulfamoyl]amino]phenoxy]-quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 170.43 mg, 46.52 mol, 17% yield) as an off-white solid. Peak-2 was assigned as stereoisomer 2. UPLC-MS (ES+): m/z 917.4 [M+H]⁺. H NMR (400 MHz, DMSO-d₆): δ 10.56 (s, 1H), 9.97 (bs, 1H), 8.89 (s, 1H), 8.16 (d, J=9.20 Hz, 1H), 7.80-7.70 (m, 2H), 7.53-7.47 (m, 2H), 7.39 (d, J=11.20 Hz, 1H), 7.22 (s, 1H), 5.35 (s, 1H), 4.29-4.20 (s, 1H), 4.03-3.89 (m, 8H), 3.51 (t, J=6.00 Hz, 2H), 3.17-3.14 (m, 2H), 2.82-2.75 (m, 4H), 2.76 (t, J=6.40 Hz, 2H), 2.68-2.67 (m, 2H), 2.59-2.50 (m, 2H), 2.37-2.32 (m, 1H), 2.12-1.76 (m, 12H).

### Synthesis D94: Synthesis of (3R)-3-[7-[2-Cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 171 and Compound 172)

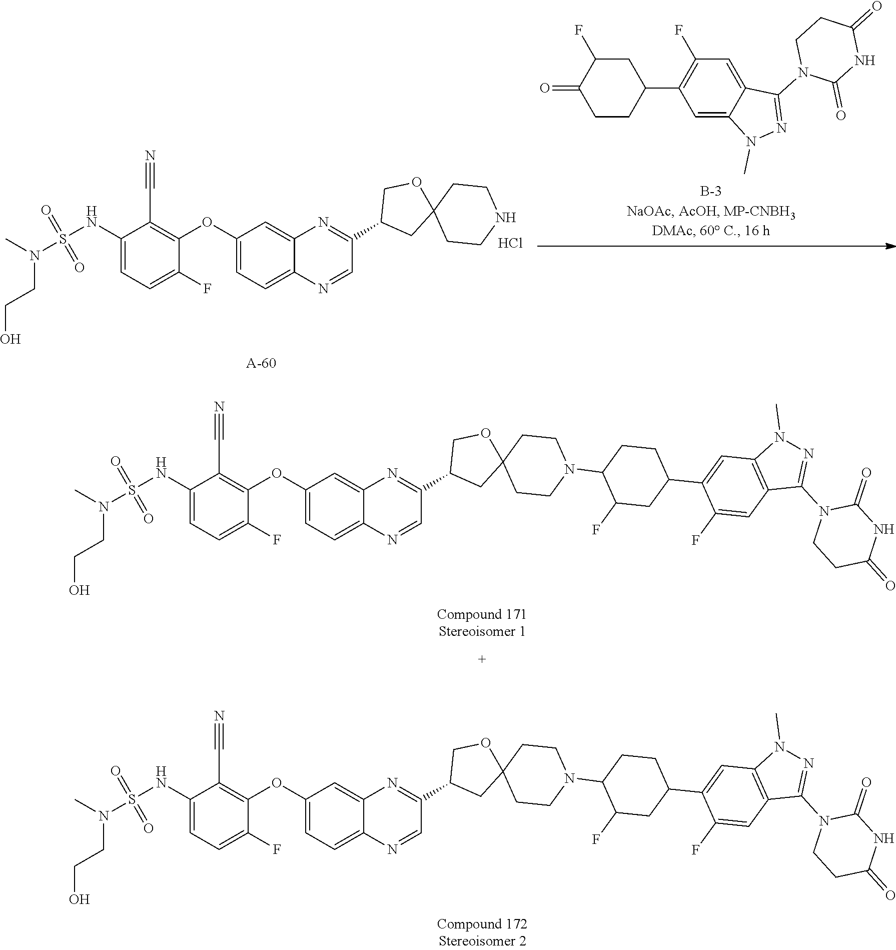 [SMILES: CN(CCO)S(=O)(=O)Nc1ccc(F)c(Oc2ccc3ncc([C@@H]4COC5(CCNCC5)C4)nc3c2)c1C#N ; Cl ; Cn1nc(N2CCC(=O)NC2=O)c2cc(F)c(C3CCC(=O)C(F)C3)cc21 ; "A-60" ; CN(CCO)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6F)CC5)C4)nc3c2)c1C#N ; CN(CCO)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6F)CC5)C4)nc3c2)c1C#N]

Into a 20 mL glass vial containing a well stirred solution of (3R)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-60) (0.350 g, 548.84 mol, HCl salt) and 1-[5-fluoro-6-(3-fluoro-4-oxo-cyclohexyl)-1-methyl-indazol-3-yl]hexahydropyrimidine-2,4-dione (B-3) (309.84 mg, 823.26 mol) in DMAc (5.0 mL) was added NaOAc (225.11 mg, 2.74 mmol) and mixture was stirred for 1 hour at room temperature. To the above mixture, AcOH (1.65 mmol, 94.17 μL) was added and stirred at room temperature for 2 hours. Subsequently, MP-CNBH₃ (0.530 g, 548.84 mol) was added and resulting reaction mixture was stirred at 60° C. for 16 hours. After completion of the reaction, reaction mixture filtered through cotton pad and filtrate was concentrated under reduced pressure to get a crude residue which was purified by preparative HPLC (Column: X-BRIDGE C18 (150 mm*19 mm*5 μm), Mobile phase A: 10 mM NH₄HCO₃ in milli-Q water; Mobile phase B: acetonitrile). The first eluting fraction (Peak-1) was lyophilized to afford Compound 171 (assigned as stereoisomer 1). Peak-1: (3R)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 171, 0.040 g, 42.45 mol, 8% yield) as a white solid. UPLC-MS (ES+): m/z 917.3 [M+H]⁺. ¹H NMR (400 MHz, DMSO-d₆): δ 10.53 (s, 1H), 9.66 (bs, 1H), 8.88 (s, 1H), 8.15 (d, J=9.20 Hz, 1H), 7.71 (dd, J=2.80, 9.20 Hz, 1H), 7.59-7.57 (m, 2H), 7.41-7.39 (m, 2H), 7.18 (d, J=2.40 Hz, 1H), 5.44-5.31 (m, 1H), 4.31-4.25 (m, 1H), 4.02-3.89 (m, 8H), 3.52 (t, J=6.40 Hz, 2H), 3.13-2.93 (m, 7H), 2.78-2.74 (m, 5H), 2.39-2.34 (m, 1H), 2.21-2.16 (m, 2H), 2.09-1.73 (m, 10H).

Similarly, the second eluted fraction (Peak-2) was lyophilized to afford Compound 172 (assigned as stereoisomer 2). Peak-2: (3R)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]-2-fluoro-cyclohexyl]-1-oxa-8-azaspiro[4.5]decane (Compound 172, 0.046 g, 49.64 mol, 9% yield) as a white solid. UPLC-MS (ES+): m/z 917.3 [M+H]. ¹H NMR (400 MHz, DMSO-d₆): δ 10.53 (s, 1H), 9.92 (bs, 1H), 8.89 (s, 1H), 8.16 (d, J=9.20 Hz, 1H), 7.77-7.70 (m, 2H), 7.53-7.47 (m, 2H), 7.39 (d, J=11.20 Hz, 1H), 7.23 (d, J=2.40 Hz, 1H), 5.25-5.13 (m, 1H), 5.00-4.40 (s, 1H), 4.29-4.20 (m, 1H), 4.03-3.90 (m, 7H), 3.52 (t, J=6.40 Hz, 2H), 3.17 (t, J=6.00 Hz, 2H), 2.83-2.67 (m, 8H), 2.62-2.54 (m, 2H), 2.37-2.32 (m, 1H), 2.20-1.50 (m, 11H).

### Synthesis D95: Synthesis of (3R)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 173 and Compound 174)

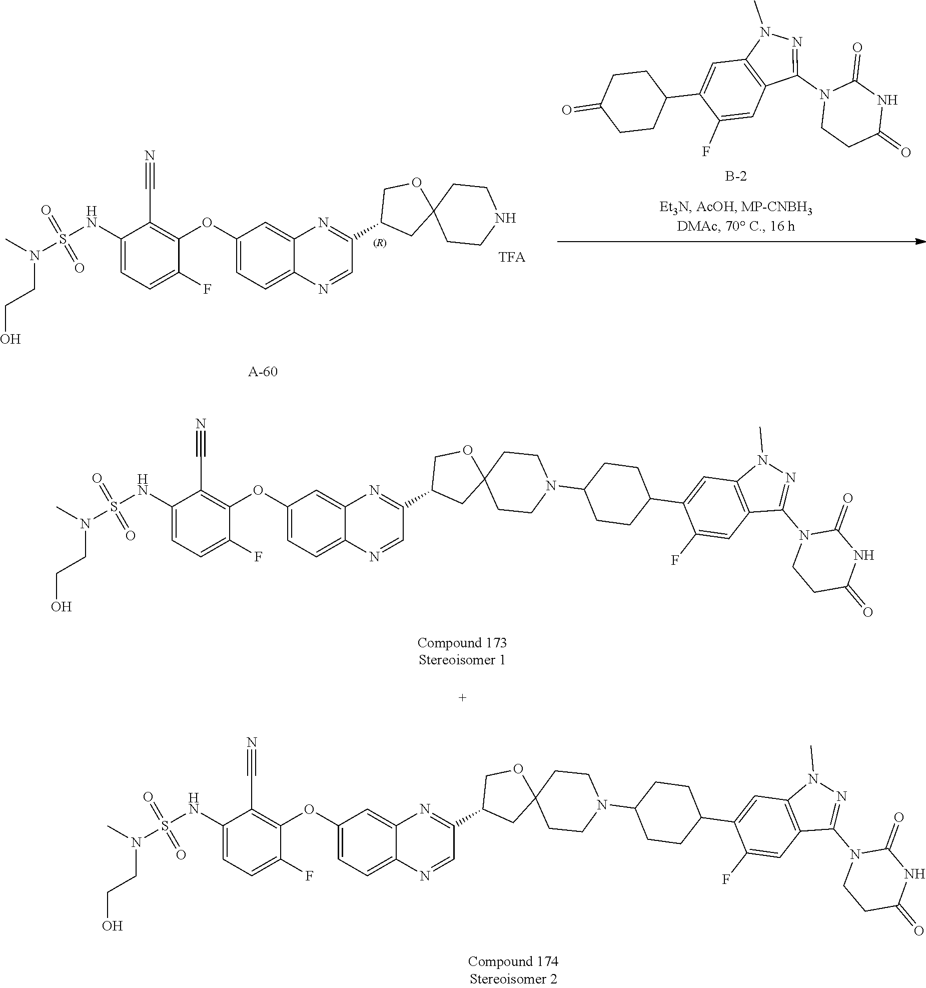

Into a 20 mL pressure vial containing a well-stirred solution of (3R)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-60) (280 mg, 417.52 μmol, TFA salt) in DMAc (4 mL) was added 1-[5-fluoro-1-methyl-6-(4-oxocyclohexyl)indazol-3-yl]hexahydropyrimidine-2,4-dione (B-2) (149.62 mg, 417.52 mol), triethylamine (1.25 mmol, 174.58 μL) at room temperature and mixture was stirred at the same temperature for 10 minutes. To the above mixture, AcOH (2.09 mmol, 119.39 L) was added, and mixture was stirred at room temperature for 2 hours. Finally, MP-CNBH₃ (280 mg) was added and resulting reaction mixture was stirred at 70° C. for 16 hours. After completion of the reaction, reaction mixture was filtered through cotton and filtrate was concentrated under reduced pressure to get a crude residue which was purified by reverse phase preparative HPLC [Column: X BRIDGE C18 (150 mm*19 min*5 μm), Mobile Phase A: 10 mM N-14HCO₃ (pH was adjusted to 10 ammonia) in H₂O, Mobile Phase B: 100% acetonitrile, flow:15 mL/min].

Fast eluting isomer (Peak-1): The fast eluting fraction (Peak-1) at t_{R}=2.06 min was lyophilized to afford (3R)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 173, 18 mg, 19.99 mol, 5% yield) as a white solid. Peak-1 was assigned as stereoisomer 1. UPLC-MS (ES+): m/z 899.3 [M+H]⁺. ¹H NMR (400 MHz, DMSO-d₆): δ 10.56 (s, 1H), 9.09 (bs, 1H), 8.86 (s, 1H), 8.14 (d, J=9.20 Hz, 1H), 7.71 (dd, J=2.80, 9.20 Hz, 1H), 7.55 (d, J=6.00 Hz, 1H), 7.40-7.37 (m, 2H), 7.31-7.29 (m, 1H), 7.09 (d, J=2.00 Hz, 1H), 4.57-4.48 (m, 1H), 4.30-4.28 (m, 1H), 4.06-3.95 (m, 5H), 3.91 (t, J=6.80 Hz, 2H), 3.50 (t, J=6.40 Hz, 2H), 3.23-3.15 (m, 2H), 3.04 (t, J=6.40 Hz, 2H), 2.90-2.82 (m, 2H), 2.76 (t, J=6.80 Hz, 2H), 2.68-2.66 (m, 4H), 2.34-2.33 (m, 1H), 2.30-2.27 (m, 1H), 2.16-2.12 (m, 2H), 2.09-1.92 (m, 7H), 1.75-1.65 (m, 4H).

Late eluting isomer (Peak-2): Similarly, late eluting fraction (Peak-2) at t_{R}=2.14 min was lyophilized to afford (3R)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 174, 35 mg, 37.18 mol, 9% yield) as a white solid. Peak-2 was assigned as stereoisomer 2. UPLC-MS (ES+): m/z 899.3 [M+H]⁺. ¹H NMR (400 MHz, DMSO-d₆): δ 10.56 (s, 1H), 8.86 (s, 1H), 8.57 (bs, 1H), 8.14 (d, J=9.20 Hz, 1H), 7.71 (dd, J=2.80, 9.00 Hz, 1H), 7.55-7.31 (m, 4H), 7.10 (bs, 1H), 4.58-4.55 (m, 1H), 4.35-4.25 (m, 1H), 4.03-3.99 (m, 5H), 3.91 (t, J=6.80 Hz, 2H), 3.58-3.48 (m, 4H), 3.42-3.37 (m, 1H), 3.22-3.05 (m, 5H), 2.76 (t, J=6.80 Hz, 2H), 2.68-2.67 (m, 2H), 2.32-2.15 (m, 2H), 2.04-1.72 (m, 13H).

### Synthesis D96: Synthesis of (3S)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 175 and Compound 176)

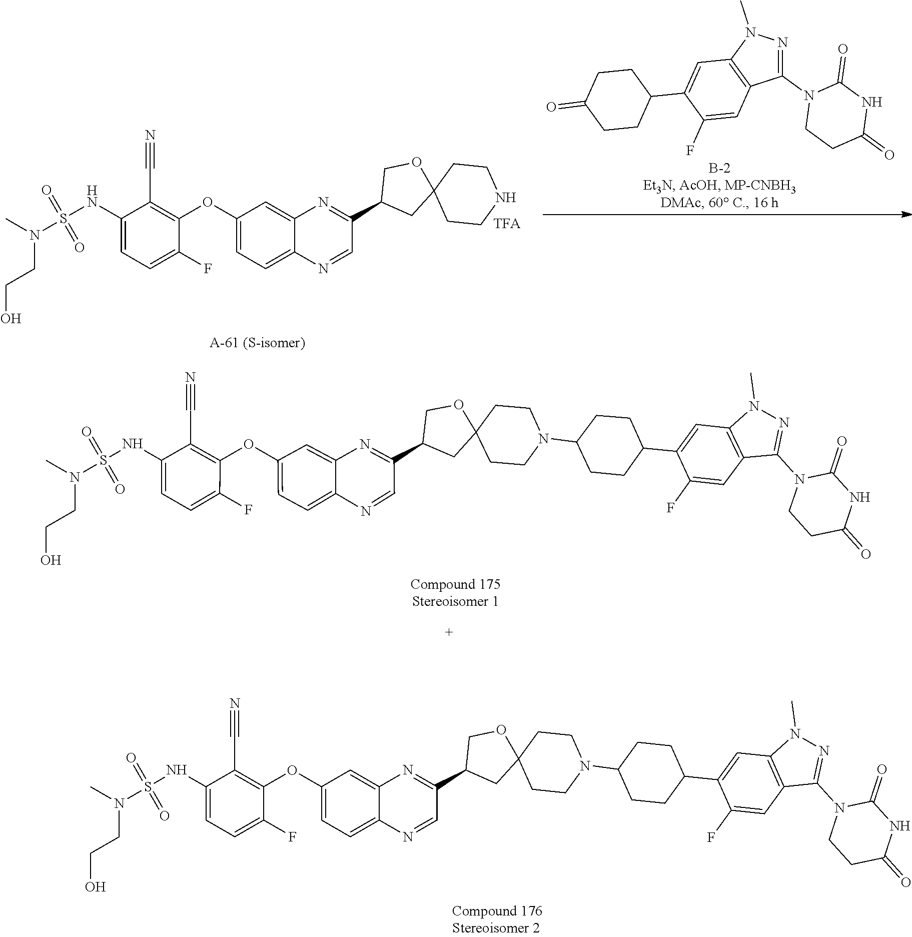 [SMILES: CN(CCO)S(=O)(=O)Nc1ccc(F)c(Oc2ccc3ncc([C@H]4COC5(CCNCC5)C4)nc3c2)c1C#N ; Cn1nc(N2CCC(=O)NC2=O)c2cc(F)c(C3CCC(=O)CC3)cc21 ; CN(CCO)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6)CC5)C4)nc3c2)c1C#N ; CN(CCO)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6)CC5)C4)nc3c2)c1C#N]

Into a 20 mL glass vial with pressure released cap containing a well-stirred solution of (3S)-3-[7-[2-cyano-6-fluoro-3-[[2-hydroxyethyl(methyl)sulfamoyl]amino]phenoxy]quinoxalin-2-yl]-1-oxa-8-azaspiro[4.5]decane (A-61) (290 mg, 272.43 mol, TFA salt) in DMAc (4 mL) was added triethylamine (817.29 mol, 113.91 μL) at room temperature and mixture was stirred at the same temperature for 5 minutes. To the above mixture, 1-[5-fluoro-1-methyl-6-(4-oxocyclohexyl)indazol-3-yl]hexahydropyrimidine-2,4-dione (B-2) (97.63 mg, 272.43 μmol) and AcOH (1.36 mmol, 77.90 μL) were added and stirred for 2 hours at room temperature. Subsequently, MP-CNBH₃ (300 mg, 272.43 μmol) was added at room temperature and resulting reaction mixture was stirred at 60° C. for 16 hours. The reaction mixture was filtered through cotton and filtrate was concentrated under reduced pressure to get a crude residue which was purified by reverse phase preparative HPLC [Column: X-BRIDGE C18 (150 mm*19 mm*5 μm), Mobile Phase A: 10 mM NH₄HCO₃ in water (pH-10), Mobile Phase B: 100% acetonitrile, flow: 14 mL/min]. Fast eluted peak in preparative HPLC (Peak-1) was lyophilized to afford Compound 175. Peak-1: (3S)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 175, 38 mg, 40.73 mol, 15% yield) as a white solid. Peak-1 was assigned as stereoisomer 1. UPLC-MS (ES+): m/z 899.2 [M+H]⁺. ¹H NMR (400 MHz, DMSO-d₆): δ 10.55 (s, 1H), 9.03 (bs, 1H), 8.86 (s, 1H), 8.14 (d, J=9.20 Hz, 1H), 7.71 (dd, J=2.80, 9.20 Hz, 1H), 7.54 (d, J=6.00 Hz, 1H), 7.40-7.29 (m, 3H), 7.10 (bs, 1H), 4.55-4.30 (m, 2H), 4.00-3.89 (m, 8H), 3.50 (t, J=6.40 Hz, 2H), 3.17-2.91 (m, 7H), 2.76 (t, J=6.80 Hz, 2H), 2.68-2.61 (m, 2H), 2.33-1.83 (m, 11H), 1.76-1.69 (m, 4H).

Similarly, late eluted peak in preparative HPLC (Peak-2) was lyophilized to afford Compound 176. Peak-2: (3S)-3-[7-[2-cyano-6-fluoro-3-[[2-hydroxyethyl(methyl)sulfamoyl]amino]phenoxy]quinoxalin-2-yl]-8-[4-[3-(2,4-dioxohexahydropyrimidin-1-yl)-5-fluoro-1-methyl-indazol-6-yl]cyclohexyl]-1-oxa-8-azaspiro[4.5]decane (Compound 176, 46 mg, 50.88 mol, 19% yield) as a white solid. Peak-2 was assigned as stereoisomer 2. UPLC-MS (ES+): m/z 899.3 [M+H]⁺. ¹H NMR (400 MHz, DMSO-d₆): δ 10.56 (s, 1H), 8.86 (s, 1H), 8.57 (bs, 1H), 8.14 (d, J=9.20 Hz, 1H), 7.69 (d, J=9.20 Hz, 1H), 7.50-7.31 (m, 4H), 7.09 (bs, 1H), 4.60-4.29 (m, 2H), 4.03-3.90 (m, 8H), 3.51-3.48 (m, 4H), 3.16-3.05 (m, 5H), 2.76 (t, J=6.80 Hz, 2H), 2.68-2.67 (m, 2H), 2.33-1.24 (m, 15H).

### Synthesis D97: Synthesis of 7-[2-cyano-3-[[ethyl(methyl)sulfamoyl]amino]-6-fluoro-phenoxy]-2-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxaline (Compound 177)

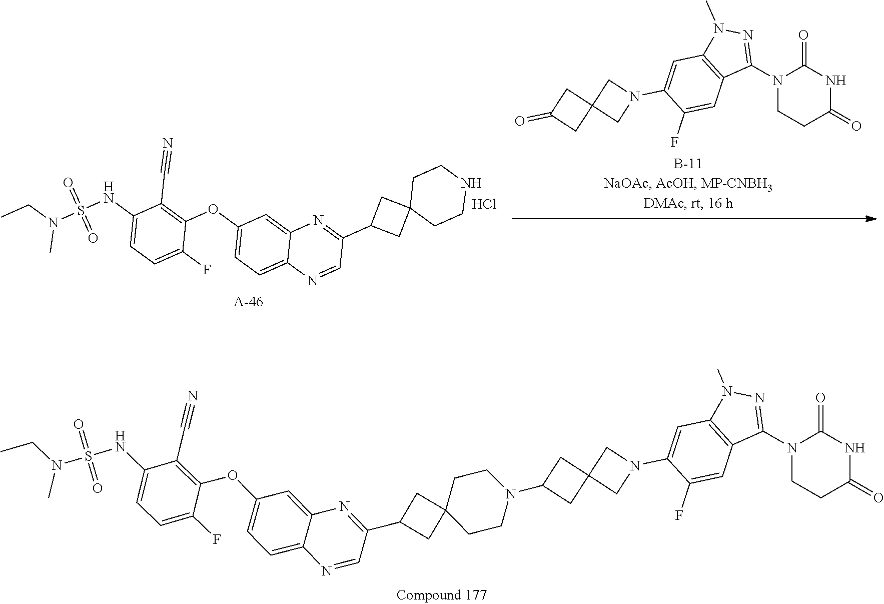

Into 20 mL glass vial containing a well stirred solution 2-(7-azaspiro[3.5]nonan-2-yl)-7-[2-cyano-3-[[ethyl(methyl)sulfamoyl]amino]-6-fluoro-phenoxy]quinoxaline (A-46) (110 mg, 172.53 mol, HCl salt) and 1-[5-fluoro-1-methyl-6-(6-oxo-2-azaspiro[3.3]heptan-2-yl)indazol-3-yl]hexahydropyrimidine-2,4-dione (B-11) (71.19 mg, 172.53 mol) in anhydrous DMAc (2 mL) was added NaOAc anhydrous (28.30 mg, 345.05 mol) followed by AcOH (172.53 mol, 9.86 L) at room temperature and mixture was stirred for 10 minutes. Subsequently, MP-CNBH₃ (110 mg, 172.53 mol) was added. The reaction mixture was stirred at room temperature for 16 hours. After completion of the starting material as indicated by UPLC, reaction mass was diluted with dichloromethane (10 mL), filtered, and concentrated under reduced pressure to afford a crude residue which was purified by preparative HPLC [Column: X-Bridge C8 (250 mm*19 mm*5 μm), Mobile phase A: 10 mM NH₄HCO₃ & NH₄OH (pH 10), Mobile phase B: 100% acetonitrile]. Fraction was lyophilized to afford 7-[2-cyano-3-[[ethyl(methyl)sulfamoyl]amino]-6-fluoro-phenoxy]-2-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxaline (Compound 177, 33 mg, 37.43 mol, 22% yield) as an off-white solid. UPLC-MS (ES+): m/z 880.3 [M+H]⁺. ¹H NMR (400 MHz, DMSO-d₆): δ 10.51 (s, 1H), 9.43 (bs, 1H), 8.78 (s, 1H), 8.13 (d, J=8.80 Hz, 1H), 7.68 (dd, J=2.80, 9.20 Hz, 1H), 7.60-7.40 (m, 1H), 7.35-7.26 (m, 2H), 7.14-7.11 (m, 1H), 6.52 (d, J=7.60 Hz, 1H), 4.03 (s, 2H), 3.97-3.86 (m, 8H), 3.03 (q, J=7.20 Hz, 3H), 2.93-2.79 (m, 2H), 2.73 (t, J=6.80 Hz, 2H), 2.68-2.62 (m, 4H), 2.34-2.23 (m, 6H), 2.05-1.45 (m, 4H), 1.04 (t, J=7.20 Hz, 3H).

### Synthesis D98: Synthesis of 3-[7-[4-[3-(2,4-dioxohexahydropyrimidin-1-yl)-5-fluoro-1-methyl-indazol-6-yl]cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 178 and Compound 179)

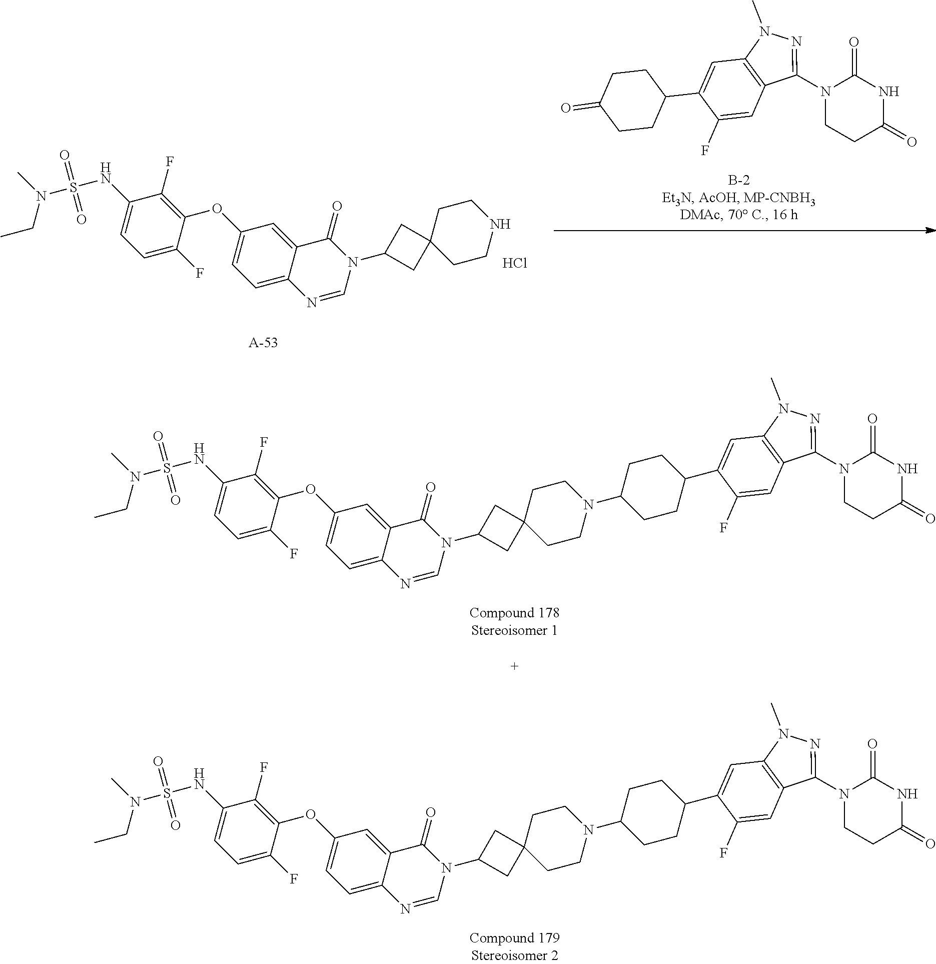

Into a 20 mL pressure vial containing a well-stirred solution of 3-(7-azaspiro[3.5]nonan-2-yl)-6-[3-[[ethyl(methyl)sulfamoyl]amino]-2,6-difluoro-phenoxy]-4-oxo-quinazoline (A-53) (250 mg, 280.68 μmol, HCl salt) in DMAc (3 mL) was added triethylamine (1.40 mmol, 195.60 μL) at room temperature under nitrogen atmosphere and mixture was stirred for 30 min. Reaction mixture was cooled to 0° C. and was added 1-[5-fluoro-1-methyl-6-(4-oxocyclohexyl)indazol-3-yl]hexahydropyrimidine-2,4-dione (B-2) (150.88 mg, 421.02 Pmol) followed by AcOH (842.03 μmol, 48.15 μL) and resulting mixture was stirred for 2 hours at room temperature. Finally, MP-CNBH₃ (250 mg, 280.68 μmol) was added, and the reaction mixture was stirred at 70° C. for 16 hours. After completion of the reaction, reaction mixture was filtered and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water (pH 10 adjusted with ammonia); Mobile phase B: acetonitrile]. The first eluting fraction (Peak-1) was lyophilized to afford Compound 178 (assigned as stereoisomer 1).

Peak-1: 3-[7-[4-[3-(2,4-dioxohexahydropyrimidin-1-yl)-5-fluoro-1-methyl-indazol-6-yl]cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 178, 19 mg, 21.58 μmol, 8% yield) as an off-white solid. UPLC-MS (ES+): m/z 876.3 [M+H]⁺. ¹H NMR (400 MHz, DMSO-d₆): δ 10.46 (bs, 1H), 8.37 (s, 1H), 7.76 (d, J=8.80 Hz, 1H), 7.63 (dd, J=3.20, 9.20 Hz, 1H), 7.53 (d, J=6.00 Hz, 1H), 7.49-7.32 (m, 3H), 7.24 (t, J=9.60 Hz, 1H), 4.90 (quint, J=8.8 Hz, 1H), 4.02 (s, 3H), 3.90 (t, J=6.40 Hz, 2H), 3.05 (q, J=7.2 Hz, 2H), 2.88-2.74 (m, 3H), 2.67 (s, 3H), 2.48-2.30 (m, 6H), 2.29-2.16 (m, 2H), 2.07-1.82 (m, 4H), 1.80-1.52 (m, 7H), 1.52-1.37 (m, 2H), 1.01 (t, J=7.20 Hz, 3H).

Similarly, the second eluted fraction (Peak-2) was lyophilized to afford Compound 179 (assigned as stereoisomer 2).

Peak-2: 3-[7-[4-[3-(2,4-dioxohexahydropyrimidin-1-yl)-5-fluoro-1-methyl-indazol-6-yl]cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 179, 18 mg, 19.67 μmol, 7% yield) as an off-white solid. UPLC-MS (ES+): m/z 876.3 [M+H]⁺. ¹H NMR (400 MHz, DMSO-d₆): δ 10.54 (s, 1H), 8.39 (s, 1H), 7.77 (d, J=8.80 Hz, 1H), 7.65 (dd, J=3.20, 8.80 Hz, 1H), 7.49-7.18 (m, 5H), 4.91 (quint, J=8.8 Hz, 1H), 4.01 (s, 3H), 3.91 (t, J=6.80 Hz, 2H), 3.08 (q, J=7.2 Hz, 2H), 3.02-2.92 (m, 1H), 2.71 (t, J=9.20 Hz, 2H), 2.67 (s, 3H), 2.35-2.29 (m, 3H), 2.28-2.21 (m, 3H), 2.15-2.04 (m, 2H), 1.97-1.80 (m, 2H), 1.79-1.49 (m, 9H), 1.01 (t, J=7.20 Hz, 3H).

### Synthesis D99: Synthesis of 7-[2-Cyano-3-[[ethyl(methyl)sulfamoyl]amino]-6-fluoro-phenoxy]-2-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxaline (Compound 180 and Compound 181)

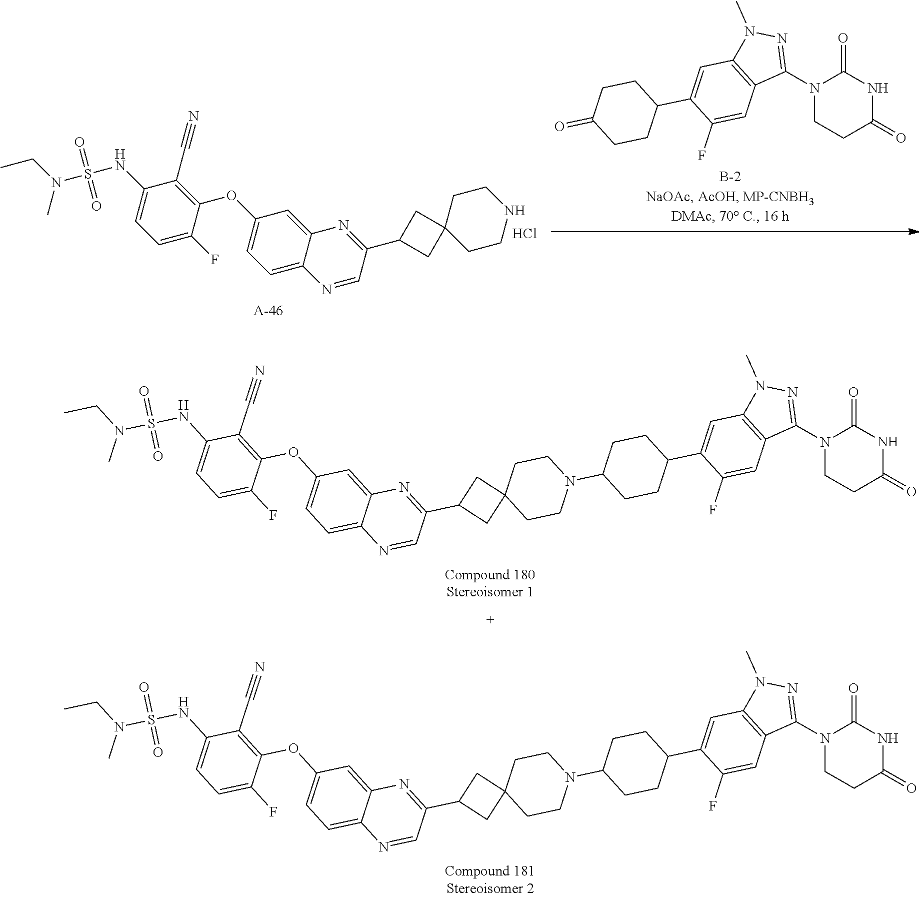

Into a 20 mL glass vial with pressure released cap containing a well-stirred solution of 2-(7-azaspiro[3.5]nonan-2-yl)-7-[2-cyano-3-[[ethyl((methyl)sulfamoyl]amino]-6-fluoro-phenoxy]quinoxaline (A-46) (250 mg, 436.67 mol, HCl salt) and 1-[5-fluoro-1-methyl-6-(4-oxocyclohexyl)indazol-3-yl]hexahydropyrimidine-2,4-dione (B-2) (234.73 mg, 655.00 mol) in DMAc (5 mL) was added anhydrous NaOAc (179.11 mg, 2.18 mmol) followed by AcOH (1.31 mmol, 74.92 μL) at room temperature under nitrogen atmosphere and stirred at room temperature for 2 hours. Finally, MP-CNBH₃ (250 mg, 436.67 mol) was added, and the reaction mixture was stirred at 70° C. for 16 hours. After the completion of the reaction, the reaction mixture was filtered and the filtrate was concentrated under reduced pressure to get a crude residue which was purified by preparative HPLC [Column: X-Bridge C8 (250 mm*19 mm*5 m), Mobile phase A: 10 mM NH₄HCO₃ & NH₄OH (pH 10) in milli-Q water; Mobile phase B: acetonitrile]. The first eluting fraction (Peak-1) was lyophilized to afford Compound 180 (assigned as stereoisomer 1).

Peak-1: 7-[2-cyano-3-[[ethyl(methyl)sulfamoyl]amino]-6-fluoro-phenoxy]-2-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxaline (Compound 180, 43 mg, 49.42 mol, 11% yield) as an off-white solid. UPLC-MS (ES+): m/z 867.3 [M+H]⁺. ¹H NMR (400 MHz, DMSO-d₆): δ 10.55 (s, 1H), 9.05 (bs, 1H), 8.78 (s, 1H), 8.14-8.11 (m, 1H), 7.68 (dd, J=2.80, 9.20 Hz, 1H), 7.54 (d, J=6.00 Hz, 1H), 7.40-7.37 (m, 2H), 7.31-7.27 (m, 1H), 7.09 (d, J=2.40 Hz, 1H), 4.00-3.89 (m, 6H), 3.32-2.90 (m, 8H), 2.76 (t, J=6.80 Hz, 2H), 2.68-2.67 (m, 3H), 2.34-2.27 (m, 4H), 2.24-2.16 (m, 2H), 2.08-2.01 (m, 3H), 1.82-1.66 (m, 7H), 1.04 (t, J=7.20 Hz, 3H).

Similarly, the second eluted fraction (Peak-2) was lyophilized to afford Compound 181 (assigned as stereoisomer 2).

Peak-2: 7-[2-cyano-3-[[ethyl(methyl)sulfamoyl]amino]-6-fluoro-phenoxy]-2-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxaline (Compound 181, 37 mg, 41.79 mol, 10% yield) as a white solid. UPLC-MS (ES+): m/z 867.3 [M+H]⁺. ¹H NMR (400 MHz, DMSO-d₆): δ 10.56 (s, 1H), 8.77 (s, 1H), 8.60 (bs, 1H), 8.14-8.11 (m, 1H), 7.68 (dd, J=2.80, 9.20 Hz, 1H), 7.50-7.30 (m, 4H), 7.10 (d, J=1.60 Hz, 1H), 4.03 (s, 3H), 3.95-3.90 (m, 3H), 3.15-2.99 (m, 7H), 2.76 (t, J=6.80 Hz, 2H), 2.68-2.60 (m, 4H), 2.34-2.25 (m, 4H), 2.08-1.81 (m, 12H), 1.04 (t, J=7.20 Hz, 3H).

### Synthesis D100: Synthesis of 3-[7-[4-[3-(2,4-dioxohexahydropyrimidin-1-yl)-5-fluoro-1-methyl-indazol-6-yl]cyclohexyl]-7-azaspiro[3.5]nonan-2-yl]-6-[3-[[ethyl(methyl)sulfamoyl]amino]-2,6-difluoro-benzoyl]-4-oxo-quinazoline (Compound 182 and Compound 183)

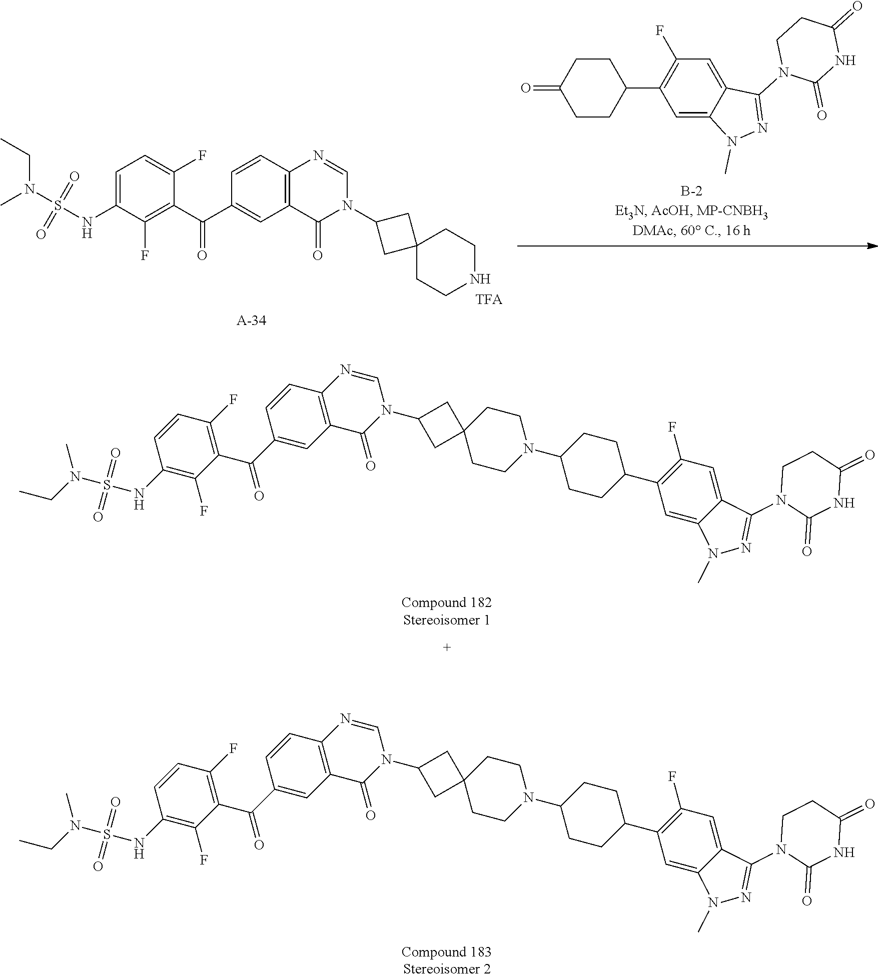 [SMILES: CCN(C)S(=O)(=O)Nc1ccc(F)c(C(=O)c2ccc3ncn(C4CC5(CCNCC5)C4)c(=O)c3c2)c1F ; Cn1nc(N2CCC(=O)NC2=O)c2cc(F)c(C3CCC(=O)CC3)cc21 ; "A-34" ; CCN(C)S(=O)(=O)Nc1ccc(F)c(C(=O)c2ccc3ncn(C4CC5(CCN(C6CCC(c7cc8c(cc7F)c(N7CCC(=O)NC7=O)nn8C)CC6)CC5)C4)c(=O)c3c2)c1F ; "TFA" ; CCN(C)S(=O)(=O)Nc1ccc(F)c(C(=O)c2ccc3ncn(C4CC5(CCN(C6CCC(c7cc8c(cc7F)c(N7CCC(=O)NC7=O)nn8C)CC6)CC5)C4)c(=O)c3c2)c1F]

Into a 20 mL glass vial with pressure released cap containing a well-stirred solution of 3-(7-azaspiro[3.5]nonan-2-yl)-6-[3-[[ethyl(methyl)sulfamoyl]amino]-2,6-difluoro-benzoyl]-4-oxo-quinazoline (A-34) (350 mg, 530.61 mol, TFA salt) in DMAc (3.05 mL) were added triethylamine (1.59 mmol, 222.17 μL) and 1-[5-fluoro-1-methyl-6-(4-oxocyclohexyl)indazol-3-yl]hexahydropyrimidine-2,4-dione (B-2) (228.18 mg, 636.73 mol) at room temperature and mixture was stirred for 10 minutes. To the above mixture AcOH (2.65 mmol, 151.72 μL) was added and the resulting mixture was stirred for 2 hours at room temperature. Subsequently, MP-CNBH₃ (350 mg, 530.61 mol) was added at the same temperature and resulting reaction mixture was stirred at 60° C. for 16 hours. After completion of the reaction, reaction mixture was filtered through cotton and filtrate was concentrated under reduced pressure to get a crude residue which was purified by reverse phase preparative HPLC [Column: X-BRIDGE C18 (150 mm*19 mm*5 μm), Mobile Phase A: 10 mM NH₄HCO₃ in water and NH₄OH (pH 10); Mobile phase B: acetonitrile]. The first eluting fraction (Peak-1 (t_{R}=5.55 min) by Prep HPLC sequence and Peak-2 by SFC sequence) was lyophilized to afford Compound 182 (assigned as stereoisomer 1).

Peak-1: 3-[7-[4-[3-(2,4-dioxohexahydropyrimidin-1-yl)-5-fluoro-1-methyl-indazol-6-yl]cyclohexyl]-7-azaspiro[3.5]nonan-2-yl]-6-[3-[[ethyl(methyl)sulfamoyl]amino]-2,6-difluoro-benzoyl]-4-oxo-quinazoline (Compound 182, 17 mg, 19.02 mol, 4% yield) as an off-white solid UPLC-MS (ES+): m/z 888.3 [M+H]⁺. ¹H NMR (400 MHz, DMSO-d₆): δ 10.55 (s, 1H), 9.78 (bs, 1H), 8.59 (s, 1H), 8.38 (d, J=2.00 Hz, 1H), 8.28 (dd, J=2.40, 8.60 Hz, 1H), 7.87 (d, J=8.80 Hz, 1H), 7.70-7.65 (m, 1H), 7.54 (d, J=6.00 Hz, 1H), 7.33-7.31 (m, 2H), 4.93-4.87 (m, 1H), 3.99 (s, 3H), 3.90 (t, J=6.80 Hz, 3H), 3.13-3.11 (m, 2H), 2.87-2.81 (m, 1H), 2.75 (t, J=6.80 Hz, 2H), 2.72 (s, 3H), 2.63-2.61 (m, 2H), 2.34-2.33 (m, 2H), 2.28-2.23 (m, 2H), 1.95-1.92 (m, 4H), 1.61-437.00 (m, 9H), 1.02 (t, J=7.20 Hz, 3H).

Similarly, the second eluting fraction (Peak-2 (t_{R}=5.59 min) by Prep HPLC sequence and Peak-1 by SFC sequence) was lyophilized to afford Compound 183 (assigned as stereoisomer 2).

Peak-2: 3-[7-[4-[3-(2,4-dioxohexahydropyrimidin-1-yl)-5-fluoro-1-methyl-indazol-6-yl]cyclohexyl]-7-azaspiro[3.5]nonan-2-yl]-6-[3-[[ethyl(methyl)sulfamoyl]amino]-2,6-difluoro-benzoyl]-4-oxo-quinazoline (Compound 183, 22 mg, 24.09 mol, 5% yield) as a pale yellow solid. UPLC-MS (ES+): m/z 888.4 [M+H]⁺. ¹H NMR (400 MHz, DMSO-d₆): δ 10.55 (s, 1H), 9.78 (bs, 1H), 8.60 (s, 1H), 8.38 (d, J=1.60 Hz, 1H), 8.29 (dd, J=2.00, 8.80 Hz, 1H), 7.87 (d, J=8.40 Hz, 1H), 7.72-7.66 (m, 1H), 7.45 (d, J=6.00 Hz, 1H), 7.39-7.32 (m, 2H), 4.96-4.87 (m, 1H), 4.02 (s, 3H), 3.91 (t, J=6.80 Hz, 2H), 3.14-3.09 (m, 2H), 3.02-2.96 (m, 1H), 2.77-2.73 (m, 6H), 2.42-2.33 (m, 3H), 2.29-2.24 (m, 3H), 2.11-2.08 (m, 2H), 1.94-1.88 (m, 2H), 1.76-1.53 (m, 8H), 1.02 (t, J=7.20 Hz, 3H).

### Synthesis D101: Synthesis of 3-[7-[4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 184) and 3-[7-[(4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 185)

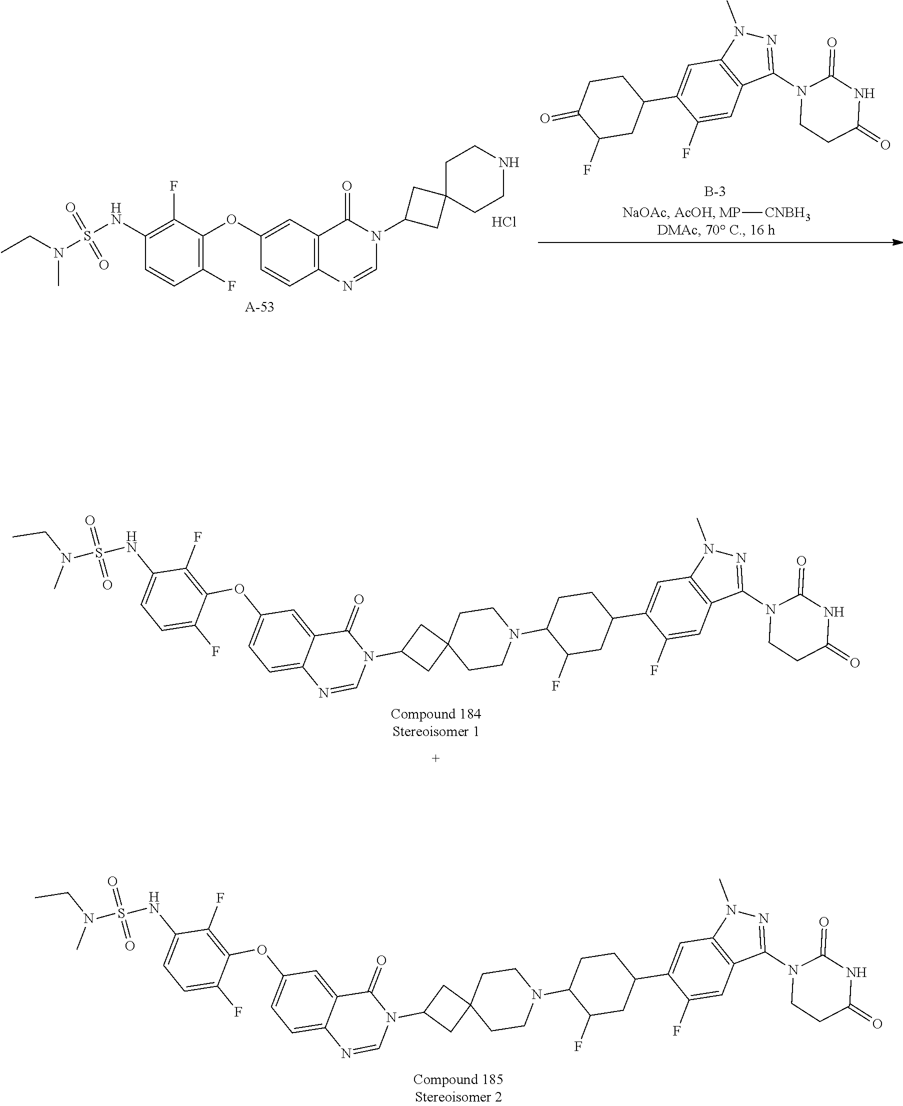

Into a 30 mL pressure vial containing a well stirred solution of 3-(7-azaspiro[3.5]nonan-2-yl)-6-[3-[[ethyl(methyl)sulfamoyl]amino]-2,6-difluoro-phenoxy]-4-oxo-quinazoline (A-53) (300 mg, 331.55 mol, HCl salt) in DMAc (5 mL) was added anhydrous NaOAc (135.99 mg, 1.66 mmol) at room temperature under nitrogen atmosphere and stirred for 20 min. Reaction mixture was cooled to 0° C. and was added 1-[5-fluoro-6-(3-fluoro-4-oxo-cyclohexyl)-1-methyl-indazol-3-yl]hexahydropyrimidine-2,4-dione (B-3) (187.17 mg, 497.32 mol) followed by AcOH (994.65 mol, 56.88 μL) and resulting mixture was stirred for 3 hours at room temperature. Subsequently, MP-CNBH₃ (300 mg, 331.55 mol) was added, and the reaction mixture was stirred at 70° C. for 16 hours. After completion of the reaction, reaction mixture was filtered and filtrate was concentrated under reduced pressure to get a crude residue, which was purified by preparative HPLC. [Column: X-SELECT CSH C18 (250 mm*19 mm*5 μm), Mobile phase A: 10 mM NH₄HCO₃ in milli-Q water; Mobile phase B: acetonitrile]. First eluting fraction (Peak-1) was concentrated under reduced pressure at 40° C. and lyophilized to afford Compound 184 (assigned as stereoisomer 1).

Peak-1: 3-[7-[(1R,4R)-4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 184, 15 mg, 16.67 mol, 5% yield) as an off white-solid. UPLC-MS (ES+): m/z 894.3 [M+H]⁺. ¹H NMR (400 MHz, DMSO-d₆): δ 10.55 (s, 1H), 9.77 (s, 1H), 8.39 (s, 1H), 7.78 (d, J=8.80 Hz, 1H), 7.66 (dd, J=2.80, 8.80 Hz, 1H), 7.59 (d, J=6.00 Hz, 1H), 7.48-7.30 (m, 4H), 5.30-5.05 (m, 1H), 4.91 (q, J=8.8 Hz, 1H), 3.99 (s, 3H), 3.90 (t, J=6.80 Hz, 2H), 3.28-3.20 (m, 1H), 3.12-3.00 (m, 2H), 2.80-2.71 (m, 5H), 2.67-2.60 (m, 2H), 2.49-2.42 (m, 3H), 2.35-2.30 (m, 2H), 2.25-2.18 (m, 2H), 2.16-2.10 (m, 1H), 2.02-1.60 (m, 9H), 1.01 (t, J=7.20 Hz, 3H).

Similarly, the second eluted fraction (Peak-2) was concentrated under reduced pressure at 40° C. and lyophilized to afford Compound 185 (assigned as stereoisomer 2).

Peak-2: 3-[7-[(1R,4S)-4-[3-(2,4-dioxohexahydropyrimidin-1-yl)-5-fluoro-1-methyl-indazol-6-yl]-2-fluoro-cyclohexyl]-7-azaspiro[3.5]nonan-2-yl]-6-[3-[[ethyl(methyl)sulfamoyl]amino]-2,6-difluoro-phenoxy]-4-oxo-quinazoline (Compound 185, 15 mg, 16.70 mol, 5% yield) as an off white-solid. UPLC-MS (ES+): m/z 894.3 [M+H]⁺. ¹H NMR (400 MHz, DMSO-d₆): δ 10.55 (s, 1H), 9.77 (s, 1H), 8.39 (s, 1H), 7.78 (d, J=8.80 Hz, 1H), 7.66 (dd, J=2.80, 8.80 Hz, 1H), 7.51 (d, J=6.00 Hz, 1H), 7.42-7.31 (m, 4H), 5.19-5.07 (m, 1H), 4.92-4.88 (m, 1H), 4.02 (s, 3H), 3.90 (t, J=6.80 Hz, 1H), 3.09-3.08 (m, 2H), 2.78-2.72 (m, 4H), 2.65-2.55 (m, 4H), 2.47-2.37 (m, 6H), 2.29-2.15 (m, 3H), 2.12-1.92 (m, 2H), 1.89-1.52 (m, 7H), 1.00 (t, J=6.80 Hz, 3H).

### Synthesis D102: Synthesis of (3R)-8-[2-[3-(2,4-dioxohexahydropyrimidin-1-yl)-5-fluoro-1-methyl-indazol-6-yl]-2-azaspiro[3.3]heptan-6-yl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 186)

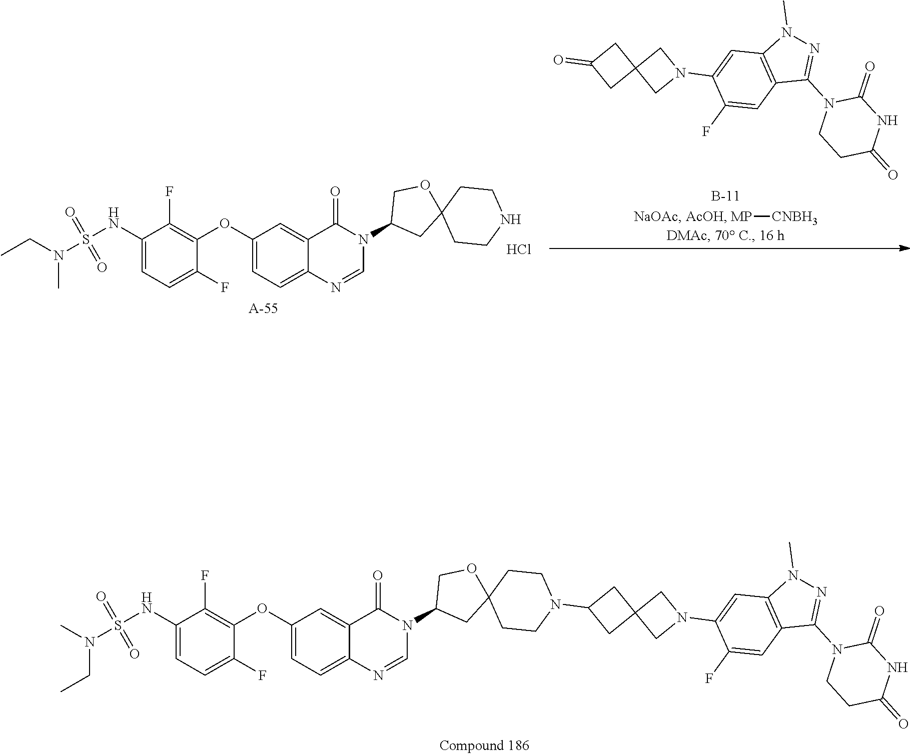

Into a 30 mL pressure vial containing a well stirred solution of (3R)-3-[6-[3-[[ethyl(methyl)sulfamoyl]amino]-2,6-difluoro-phenoxy]-4-oxo-quinazolin-3-yl]-1-oxa-8-azaspiro[4.5]decane (A-55) (150.00 mg, 255.95 mol, HCl salt) in DMAc (3.01 mL) was added anhydrous NaOAc (62.99 mg, 767.85 mol) at room temperature under nitrogen atmosphere and mixture was stirred for 20 min. Reaction mixture was cooled to 0° C. and was added 1-[5-fluoro-1-methyl-6-(6-oxo-2-azaspiro[3.3]heptan-2-yl)indazol-3-yl]hexahydropyrimidine-2,4-dione (B-11) (104.56 mg, 281.55 mol) followed by AcOH (1.28 mmol, 73.89 μL) and resulting mixture was stirred for 2 hours at room temperature. Finally, MP-CNBH₃ (150 mg, 255.95 mol) was added, and the reaction mixture was stirred at 70° C. for 16 hours. After completion of the reaction, reaction mixture was filtered and filtrate was concentrated under reduced pressure to get a crude residue, which was purified by reverse phase column chromatography (Redisep R_{f} Gold®reversed-phase C18-teledyne ISCO, 120 g), [Mobile phase A: 10 mM NH₄HCO₃ in milli-Q water; Mobile phase B: acetonitrile]. Fractions were lyophilized to afford (3R)-8-[2-[3-(2,4-dioxohexahydropyrimidin-1-yl)-5-fluoro-1-methyl-indazol-6-yl]-2-azaspiro[3.3]heptan-6-yl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 186, 60 mg, 64.67 mol, 25% yield) as an off-white solid. UPLC-MS (ES+): m/z 905.3 [M+H]⁺. ¹H NMR (400 MHz, DMSO-d₆): δ 10.50 (s, 1H), 9.77 (s, 1H), 8.32 (s, 1H), 7.77 (d, J=8.80 Hz, 1H), 7.67 (dd, J=3.20, 9.20 Hz, 1H), 7.48-7.30 (m, 3H), 7.25 (d, J=12.80 Hz, 1H), 6.49 (d, J=7.20 Hz, 1H), 5.25-5.22 (m, 1H), 4.13-4.03 (m, 2H), 4.12-3.94 (m, 2H), 3.92-3.83 (m, 7H), 3.12-3.03 (m, 2H), 2.77-2.70 (m, 6H), 2.66-2.56 (m, 3H), 2.33-2.16 (m, 4H), 2.07-1.95 (m, 3H), 1.79-1.57 (m, 4H), 1.03 (t, J=7.20 Hz, 3H).

### Synthesis D103: Synthesis of 7-[2-cyano-6-fluoro-3-[[2-hydroxyethyl(methyl)sulfamoyl]amino]phenoxy]-2-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxaline (Compound 187)

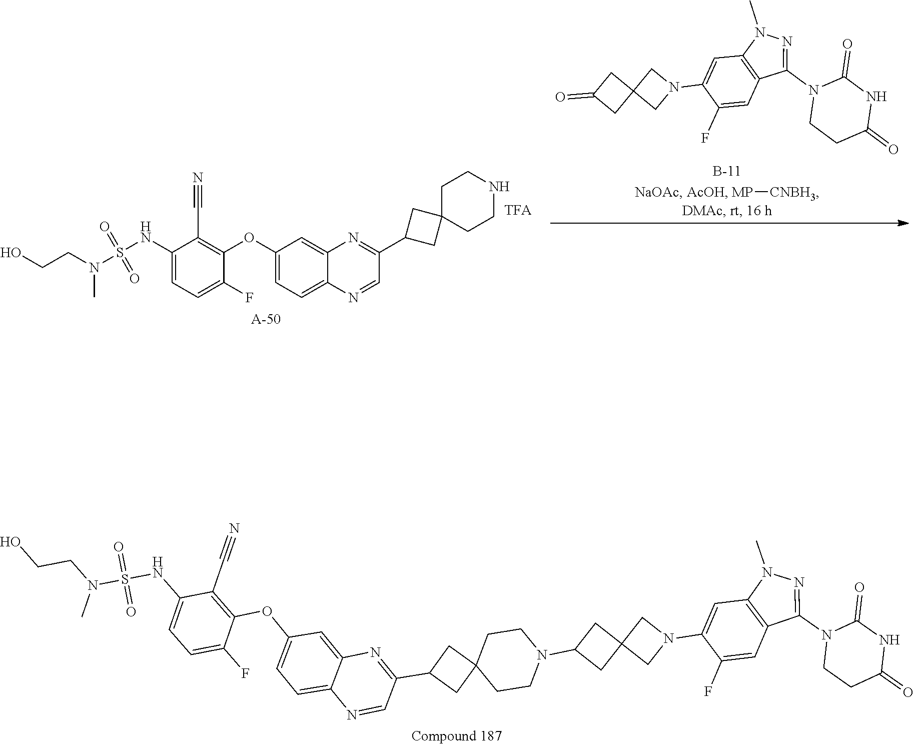

Into a 10 mL pressure vial containing a well stirred solution 2-(7-azaspiro[3.5]nonan-2-yl)-7-[2-cyano-6-fluoro-3-[[2-hydroxyethyl(methyl)sulfamoyl]amino]phenoxy]quinoxaline (A-50) (120 mg, 124.65 mol, TFA salt) and 1-[5-fluoro-1-methyl-6-(6-oxo-2-azaspiro[3.3]heptan-2-yl)indazol-3-yl]hexahydropyrimidine-2,4-dione (B-11) (56.58 mg, 137.12 mol) in anhydrous DMAc (2.0 mL) was added NaOAc (20.45 mg, 249.30 mol) followed by AcOH (124.65 mol, 7.13 μL) at room temperature and stirred for 10 minutes. Finally, MP-CNBH₃ (120 mg, 124.65 mol) was added and stirred at room temperature for 16 hours. After completion of the reaction, reaction mixture was diluted with dichloromethane (10 mL), filtered on cotton pad and filtrate was concentrated under reduced pressure to afford crude which was purified by preparative HPLC method [Column: X-Bridge (150 mm*19 mm*5 μm), Mobile phase A: 10 mM NH₄HCO₃ & NH₄OH (pH 10) in milli-Q water; Mobile phase B: acetonitrile]. The fractions were lyophilized to afford 7-[2-cyano-6-fluoro-3-[[2-hydroxyethyl(methyl)sulfamoyl]amino]phenoxy]-2-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxaline (Compound 187, 27 mg, 29.72 mol, 24% yield) as an off-white solid. UPLC-MS(ES+): m/z 896.9 [M+H]⁺. ¹H NMR (400 MHz, DMSO-d₆): δ 10.51 (s, 1H), 9.25 (bs, 1H), 8.78 (s, 1H), 8.13 (d, J=9.20 Hz, 1H), 7.68 (dd, J=2.80, 9.20 Hz, 1H), 7.51-7.48 (m, 1H), 7.34-7.26 (m, 2H), 7.13 (s, 1H), 6.53 (d, J=7.20 Hz, 1H), 4.65-4.51 (m, 1H), 4.03 (s, 2H), 3.94-3.86 (m, 9H), 3.51 (t, J=6.40 Hz, 4H), 3.07 (t, J=6.00 Hz, 3H), 2.75-2.68 (m, 6H), 2.38-2.20 (m, 6H), 2.18-1.40 (m, 6H).

### Synthesis D104: Synthesis of 3-[7-[2-[4-[(2,6-dioxo-3-piperidyl)amino]-2-fluoro-phenyl]-2-azaspiro[3.3]heptan-6-yl]-7-azaspiro[3.5]nonan-2-yl]-6-[3-[[ethyl(methyl)sulfamoyl]amino]-2,6-difluoro-phenoxy]-4-oxo-quinazoline (Compound 188)

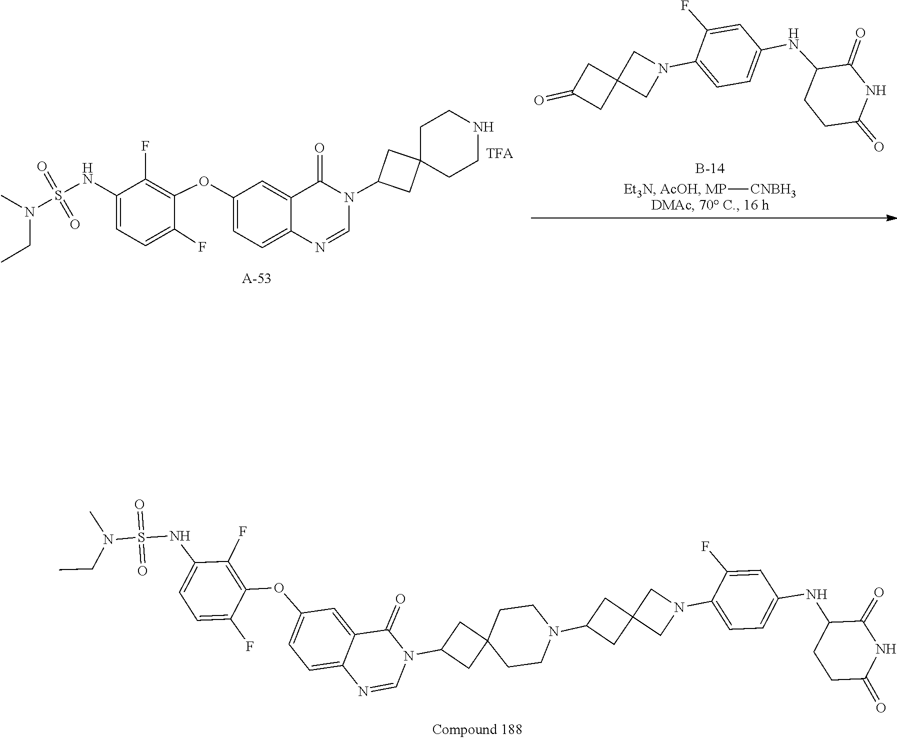

Into a 20 mL pressure vial containing a well-stirred solution of 3-(7-azaspiro[3.5]nonan-2-yl)-6-[3-[[ethyl(methyl)sulfamoyl]amino]-2,6-difluoro-phenoxy]-4-oxo-quinazoline 1 (A-53) (200 mg, 221.03 mol, HCl salt) in DMAc (4 mL) was added triethylamine (663.10 mol, 92.42 L) at room temperature and stirred for 5 minutes. Subsequently, AcOH (442.07 mol, 25.28 μL) and 3-[3-fluoro-4-(6-oxo-2-azaspiro[3.3]heptan-2-yl)anilino]piperidine-2,6-dione 2 (B-14) (132.36 mg, 331.55 mol) were added and stirred at room temperature for 2 hours. Finally, MP-CNBH₃ (200 mg, 221.03 mol) was added, and the resulting reaction mixture was stirred at 70° C. for 16 hours. After completion of the reaction, reaction mixture was diluted with dichloromethane (10 mL), filtered and filtrate was concentrated under reduced pressure to afford crude, which was purified by preparative HPLC [Column: X-Bridge C18 (150 mm*21 mm*5 μm), Mobile phase A: 10 mM NH₄HCO₃ in milli-Q water; Mobile phase B: acetonitrile]. The fraction was lyophilized to afford 3-[7-[2-[4-[(2,6-dioxo-3-piperidyl)amino]-2-fluoro-phenyl]-2-azaspiro[3.3]heptan-6-yl]-7-azaspiro[3.5]nonan-2-yl]-6-[3-[[ethyl(methyl)sulfamoyl]amino]-2,6-difluoro-phenoxy]-4-oxo-quinazoline (Compound 188, 5.5 mg, 6.36 mol, 3% yield) as an off-white solid. UPLC-MS (ES+): m/z 849.3 [M+H]⁺. ¹H NMR (400 MHz, DMSO-d₆): δ 10.75 (s, 1H), 9.71 (bs, 1H), 8.38 (s, 1H), 7.77 (d, J=8.80 Hz, 1H), 7.66 (dd, J=3.20, 8.80 Hz, 1H), 7.42-7.31 (m, 3H), 6.49-6.38 (m, 3H), 5.53 (d, J=7.20 Hz, 1H), 4.91-4.87 (m, 1H), 4.20-4.18 (m, 1H), 3.72 (s, 2H), 3.61 (s, 2H), 3.08 (q, J=6.80 Hz, 2H), 2.73-2.68 (m, 4H), 2.59-2.58 (m, 1H), 2.34-2.21 (m, 8H), 2.18-2.06 (m, 2H), 1.96-1.80 (m, 4H), 1.65-1.55 (m, 4H), 1.00 (t, J=7.20 Hz, 3H).

### Synthesis D105: Synthesis of 6-[2-cyano-3-[[ethyl(methyl)sulfamoyl]amino]-6-fluoro-phenoxy]-3-[(3R)-8-[(4S)-4-[4-[(2,6-dioxo-3-piperidyl)amino]-2-fluoro-phenyl]-3,3-difluoro-1-piperidyl]-1-oxaspiro[4.5]decan-3-yl]-4-oxo-quinazoline (Compound 189 and Compound 190)

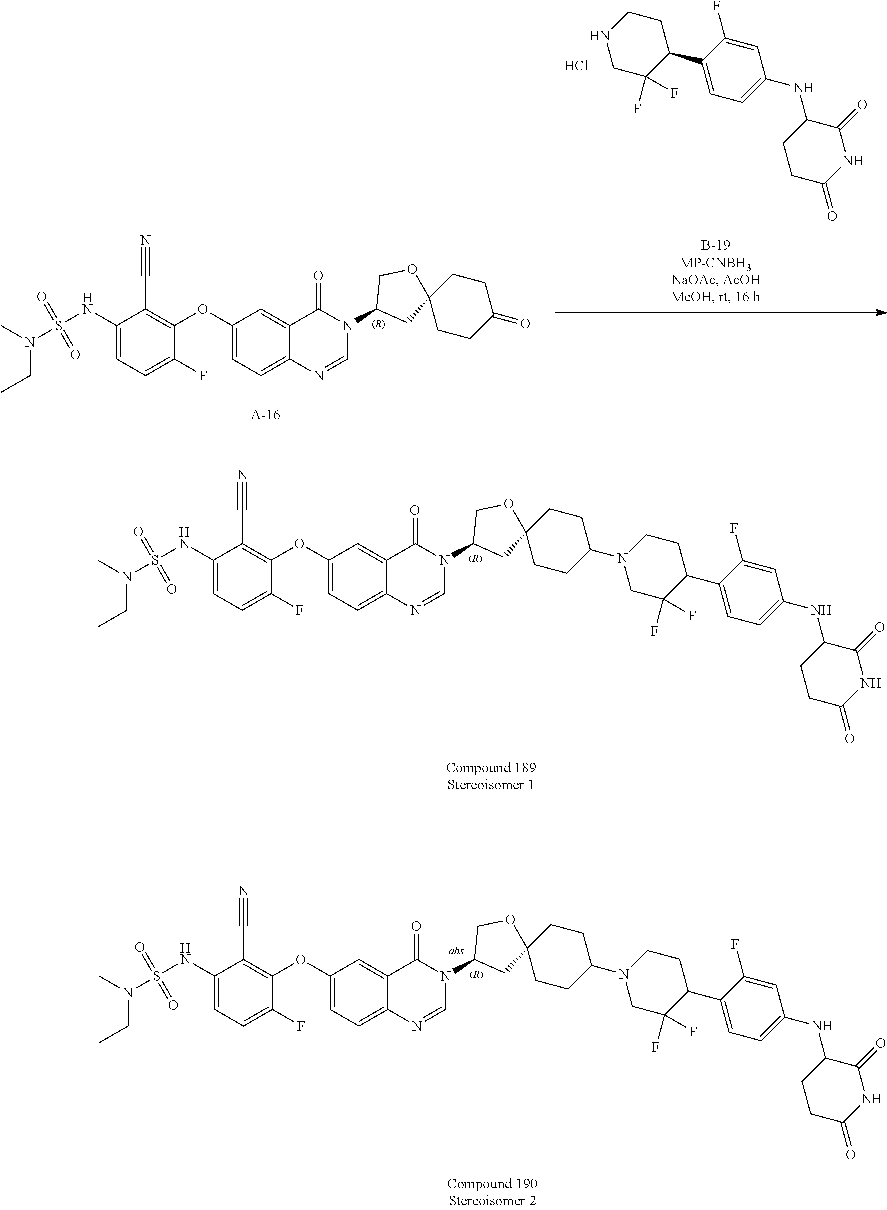

Into a 20 mL pressure vial containing a well-stirred solution of 3-[4-[(4S)-3,3-difluoro-4-piperidyl]-3-fluoro-anilino]piperidine-2,6-dione (B-19) (165.81 mg, 438.90 mol, HCl Salt) in methanol (5 mL) was added anhydrous NaOAc (180.02 mg, 2.19 mmol) at room temperature under nitrogen atmosphere and stirred for 20 minutes. Reaction mixture was cooled to 0° C. and 6-[2-cyano-3-[[ethyl(methyl)sulfamoyl]amino]-6-fluoro-phenoxy]-4-oxo-3-[(3R)-8-oxo-1-oxaspiro[4.5]decan-3-yl]quinazoline (A-16) (250 mg, 438.90 mol) followed by AcOH (438.90 mol, 25.12 μL) were added and stirred at room temperature for 3 hours. Subsequently, MP-CNBH₃ (250 mg, 438.90 mol) was added, and the reaction mixture was stirred at room temperature for 16 hours. After completion of the reaction, reaction mixture was filtered and the filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile). The fractions were lyophilized to afford 100 mg of mixture of R and Sisomers. Further R and S isomers were separated by chiral SFC.

Chiral SFC separation: Above 100 mg of isomeric mixture was purified by chiral SFC following the below method.

### Method of Analysis for Chiral SFC Separation

Instrument: PIC-22-017
Column: LUXA1-(250*30) mm, 5 m
Mobile Phase: CO₂: 0.2% FA in IPA: CAN (1:1) [75:25]
Total Flow: 100 mL/min
Back pressure: 100 bar
Wavelength: 220 nm
Cycle time: 6 min

Fast eluting isomer (Peak-1): Fast eluting compound (Peak-1) was collected and concentrated under reduced pressure to afford 6-[2-cyano-3-[[ethyl(methyl)sulfamoyl]amino]-6-fluoro-phenoxy]-3-[(3R)-8-[(4S)-4-[4-[(2,6-dioxo-3-piperidyl)amino]-2-fluoro-phenyl]-3,3-difluoro-1-piperidyl]-1-oxaspiro[4.5]decan-3-yl]-4-oxo-quinazoline (Compound 189, 18.12 mg, 19.84 mol, 18% yield) as an off-white solid. Peak-1 was assigned as stereoisomer 1.

UPLC-MS (ES+): m/z 895.2 [M+H]⁺. ¹H NMR (400 MHz, DMSO-d₆): δ 10.79 (s, 1H), 10.17 (s, 1H), 8.35 (d, J=4.40 Hz, 1H), 7.80-7.70 (m, 2H), 7.69 (dd, J=2.80, 8.8 Hz, 1H), 7.48-7.47 (m, 1H), 7.38-7.35 (m, 1H), 7.08-7.04 (m, 1H), 6.52-6.44 (m, 2H), 6.16-6.14 (m, 1H), 5.25 (bs, 1H), 4.34-4.33 (m, 1H), 4.10-4.07 (m, 2H), 3.25-3.00 (m, 4H), 2.99-2.90 (m, 1H), 2.85-2.70 (m, 4H), 2.65-2.55 (m, 2H), 2.43-2.38 (m, 2H), 2.30-2.20 (m, 1H), 2.10-1.45 (m, 11H), 1.42-1.28 (m, 2H), 1.06 (t, J=7.20 Hz, 3H).

Late eluting isomer (Peak-2): Late eluting compound (Peak-2) was collected and concentrated under reduced pressure to afford 6-[2-cyano-3-[[ethyl(methyl)sulfamoyl]amino]-6-fluoro-phenoxy]-3-[(3R)-8-[(4S)-4-[4-[(2,6-dioxo-3-piperidyl)amino]-2-fluoro-phenyl]-3,3-difluoro-1-piperidyl]-1-oxaspiro[4.5]decan-3-yl]-4-oxo-quinazoline (Compound 190, 22.24 mg, 24.55 mol, 22% yield) as an off-white solid. Peak-2 was assigned as stereoisomer 2. UPLC-MS (ES+): m/z 895.2 [M+H]⁺. ¹H NMR (400 MHz, DMSO-d₆): δ 10.79 (s, 1H), 10.17 (s, 1H), 8.35 (d, J=4.00 Hz, 1H), 7.80-7.83 (m, 2H), 7.69 (dd, J=2.80, 8.8 Hz, 1H), 7.48 (dd, J=3.60, 8.80 Hz, 1H), 7.37-7.35 (m, 1H), 7.10-7.04 (m, 1H), 6.52-6.44 (m, 2H), 6.15 (d, J=3.60 Hz, 1H), 5.25 (bs, 1H), 4.34-4.33 (m, 1H), 4.10-4.07 (m, 2H), 3.17-3.12 (m, 4H), 2.99-2.90 (m, 1H), 2.85-2.70 (m, 4H), 2.65-2.55 (m, 1H), 2.43-2.38 (m, 2H), 2.30-2.20 (m, 1H), 2.10-1.45 (m, 11H), 1.42-1.28 (m, 2H), 1.06 (t, J=7.20 Hz, 3H).

### Synthesis D106: Synthesis of 6-[2-cyano-3-[[ethyl(methyl)sulfamoyl]amino]-6-fluoro-phenoxy]-3-[(3R)-8-[6-[4-[(2,6-dioxo-3-piperidyl)amino]-2-fluoro-phenyl]-2,6-diazaspiro[3.3]heptan-2-yl]-1-oxaspiro[4.5]decan-3-yl]-4-oxo-quinazoline (Compound 191 and Compound 192)

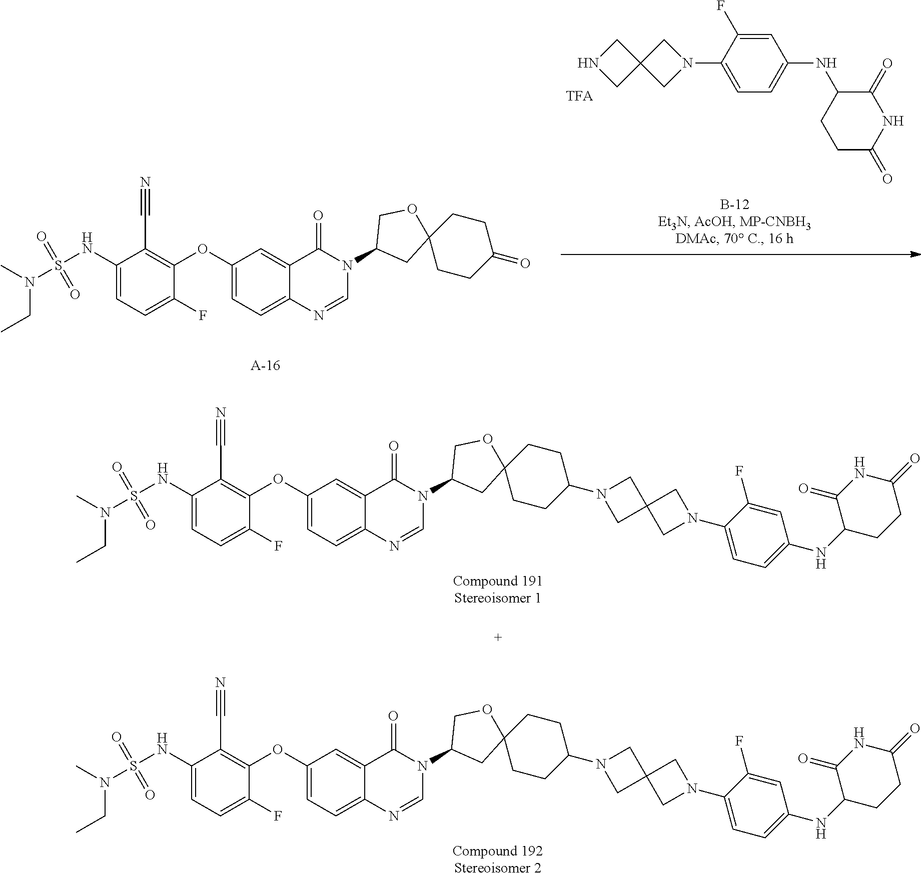

Into a 40 mL pressure glass vial containing a well stirred solution of 6-[2-cyano-3-[[ethyl(methyl)sulfamoyl]amino]-6-fluoro-phenoxy]-4-oxo-3-[(3R)-8-oxo-1-oxaspiro[4.5]decan-3-yl]quinazoline (A-16) (400 mg, 702.24 mol) and 3-[4-(2,6-diazaspiro[3.3]heptan-2-yl)-3-fluoro-anilino]piperidine-2,6-dione (B-12) (394.72 mg, 912.92 mol, TFA salt) in DMAc (6 mL) was added triethylamine (3.51 mmol, 489.39 μL) stirred at room temperature for 10 minutes. Subsequently, AcOH (2.81 mmol, 160.64 μL) was added and stirred at room temperature for 2 hours. Finally, MP-CNBH₃ (400 mg, 702.24 μmol) was added and stirred at 70° C. for 16 hours. After completion of the reaction, reaction mixture was diluted with dichloromethane (10 mL), filtered through cotton pad and filtrate was concentrated under reduced pressure to get crude which was purified by reverse phase by using RediSep R_{f} Gold® 100 g column and [Mobile phase A: 10 mM NH₄HCO₃ in milli-Q water; Mobile phase B: acetonitrile] Eluted fractions were lyophilized to afford 140 mg of racemic mixture (UPLC-MS, 98%) as an off-white solid.

Chiral SFC separation: Above isomeric mixture was purified by SFC following the below method.
Instrument: PIC 22-016
Column: Diol(250*30)mm, 5 μm
Mobile Phase: C02:0.5% IPAm in IPA [60:40]
Total Flow: 120 mL/min
Back pressure: 120 bar
Wavelength: 254 nm
Cycle time: 9 min

Fast eluting isomer (Peak-1): Fast eluting compound (Peak-1, t_{R}=3.72 min) was collected and concentrated under reduced pressure to get Peak-1 which is not pure. Further purified by reverse phase by using RediSep R_{f} Gold® 100 g column and [Mobile phase A: 10 mM NH₄HCO₃ in milli-Q water; Mobile phase B: acetonitrile] Eluted fractions were lyophilized to afford 6-[2-cyano-3-[[ethyl(methyl)sulfamoyl]amino]-6-fluoro-phenoxy]-3-[(3R)-8-[6-[4-[(2,6-dioxo-3-piperidyl)amino]-2-fluoro-phenyl]-2,6-diazaspiro[3.3]heptan-2-yl]-1-oxaspiro[4.5]decan-3-yl]-4-oxo-quinazoline (Compound 191, 19 mg, 21.43 mol, 3% yield) as an off-white solid. Peak-1 was assigned as stereoisomer 1. UPLC-MS (ES+): m/z 872.1 [M+H]⁺. ¹H NMR (400 MHz, DMSO-d₆): δ 10.76 (s, 1H), 9.94 (s, 1H), 8.32 (s, 1H), 7.77 (d, J=8.80 Hz, 1H), 7.66 (dd, J=2.80, 8.80 Hz, 1H), 7.55-7.51 (m, 1H), 7.35-7.34 (m, 2H), 6.51-6.37 (m, 3H), 5.60 (d, J=7.60 Hz, 1H), 5.28-5.25 (m, 1H), 4.23-4.05 (m, 7H), 3.91-3.81 (m, 4H), 3.06-3.05 (m, 2H), 2.72-2.66 (m, 3H), 2.59-2.58 (m, 2H), 2.40-2.37 (m, 2H), 2.09-2.01 (m, 2H), 1.92-1.82 (m, 5H), 1.60-1.55 (m, 1H), 1.43-1.40 (m, 1H), 1.24-1.22 (m, 2H), 1.04 (t, J=7.20 Hz, 3H).

Late eluting isomer (Peak-2): Late eluting compound (Peak-2, t_{R}=4.30 min) was collected and concentrated under reduced pressure to afford Peak-2 which is not pure. Further purified by following SFC method,
Instrument: PIC 22-016
Column: I Cellulose J(250*30)mm, 5 m
Mobile Phase: CO₂: methanol: ACN [65:35]
Total Flow: 100 ml/min
Back pressure: 100 bar
Wavelength: 220 nm
Cycle time: 8 min

Collected fractions were concentrated under reduced pressure to afford 6-[2-cyano-3-[[ethyl(methyl)sulfamoyl]amino]-6-fluoro-phenoxy]-3-[(3R)-8-[6-[4-[(2,6-dioxo-3-piperidyl)amino]-2-fluoro-phenyl]-2,6-diazaspiro[3.3]heptan-2-yl]-1-oxaspiro[4.5]decan-3-yl]-4-oxo-quinazoline (Compound 192, 25 mg, 28.51 mol, 4.06% yield, 99% purity) as an off-white solid. Peak-2 was assigned as stereoisomer 2. UPLC-MS (ES+): m/z 872.3 [M+H]⁺. ¹H NMR (400 MHz, DMSO-d₆): δ 10.76 (s, 1H), 9.83 (s, 1H), 8.30 (s, 1H), 7.75 (d, J=9.20 Hz, 1H), 7.64 (dd, J=3.20, 9.00 Hz, 1H), 7.41-7.29 (m, 3H), 6.52-6.38 (m, 3H), 5.60 (d, J=7.60 Hz, 1H), 5.28-5.24 (m, 1H), 4.32-3.87 (m, 11H), 3.02-3.00 (m, 2H), 2.72-2.68 (m, 1H), 2.59-2.56 (m, 5H), 2.30-2.26 (m, 1H), 2.09-2.06 (m, 2H), 1.92-1.72 (m, 5H), 1.55-1.33 (m, 4H), 1.03 (t, J=7.20 Hz, 3H).

### Synthesis D107: Synthesis of (3R)-8-[2-[4-[(2,6-dioxo-3-piperidyl)amino]-2-fluoro-phenyl]-2-azaspiro[3.3]heptan-6-yl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 193)

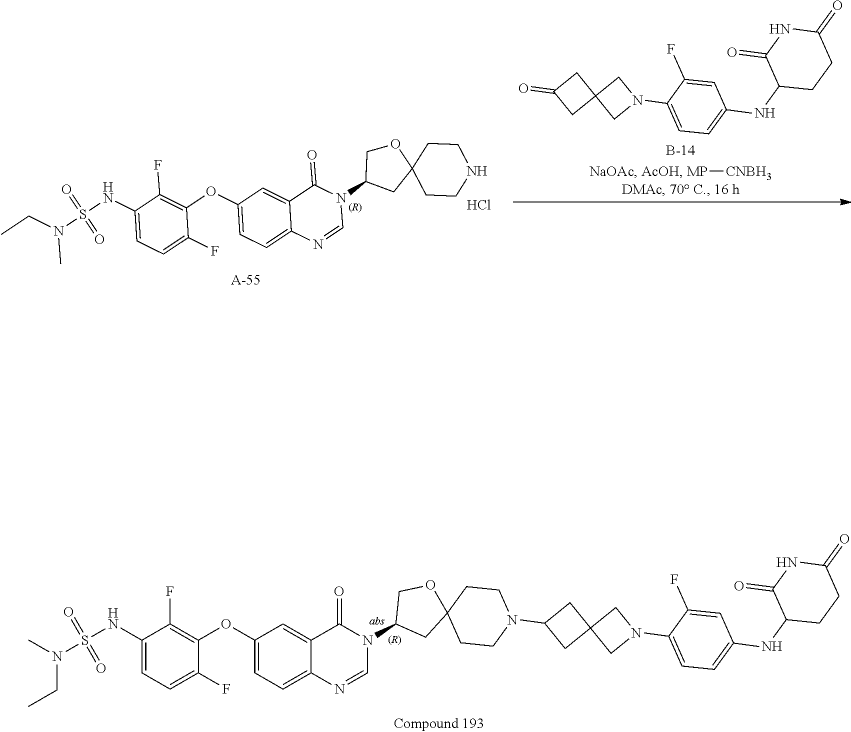

Into a 8 mL pressure vial containing a well-stirred solution of (3R)-3-[6-[3-[[ethyl(methyl)sulfamoyl]amino]-2,6-difluoro-phenoxy]-4-oxo-quinazolin-3-yl]-1-oxa-8-azaspiro[4.5]decane (A-55) (100 mg, 170.63 mol, HCl salt) in DMAc (3 mL) was added NaOAc (41.99 mg, 511.90 mol) at room temperature under nitrogen atmosphere and mixture was stirred for 20 min. Reaction mixture was cooled to 0° C. and was added 3-[3-fluoro-4-(6-oxo-2-azaspiro[3.3]heptan-2-yl)anilino]piperidine-2,6-dione (B-14) (84.81 mg, 255.95 mol) followed by AcOH (853.17 mol, 48.79 μL) and resulting mixture was stirred for 3 hours at room temperature. Finally, MP-CNBH₃ (100 mg, 170.63 mol) was added, and the reaction mixture was stirred at 70° C. for 16 hours. After the completion of the reaction, reaction mixture was filtered and filtrate was concentrated under reduced pressure to get a crude residue which was purified by reverse phase columnchromatography (Redisep R_{f}Gold®reversed-phase C18-teledyne ISCO, 120 g), [Mobile phase A: NH₄HCO₃ in milli-Q water; Mobile phase B: acetonitrile]. Fractions were lyophilized to afford (3R)-8-[2-[4-[(2,6-dioxo-3-piperidyl)amino]-2-fluoro-phenyl]-2-azaspiro[3.3]heptan-6-yl]-3-[6-[3-[[ethyl(methyl)sulfamoyl]amino]-2,6-difluoro-phenoxy]-4-oxo-quinazolin-3-yl]-1-oxa-8-azaspiro[4.5]decane (Compound 193, 11 mg, 12.52 mol, 7% yield) as a pale brown solid. UPLC-MS (ES+): m/z 865.3 [M+H]⁺. ¹H NMR (400 MHz, DMSO-d₆): δ 10.75 (s, 1H), 9.76 (bs, 1H), 8.32 (s, 1H), 7.77 (d, J=8.80 Hz, 1H), 7.67 (dd, J=3.20, 9.00 Hz, 1H), 7.44-7.32 (m, 3H), 6.47 (dd, J=2.00, 15.20 Hz, 1H), 6.40-6.31 (m, 2H), 5.53 (d, J=7.60 Hz, 1H), 5.24 (bs, 1H), 4.23-3.98 (m, 3H), 3.72 (s, 2H), 3.61 (s, 2H), 3.09 (q, J=7.20 Hz, 2H), 2.80-2.70 (m, 4H), 2.63-2.58 (m, 2H), 2.31-2.01 (m, 7H), 2.01-1.90 (m, 3H), 1.48-0.90 (m, 6H), 1.01 (t, J=7.20 Hz, 3H).

### Synthesis D108: Synthesis of (3R)-3-[6-[2-cyano-3-[[ethyl(methyl)sulfamoyl]amino]-6-fluoro-phenoxy]-4-oxo-quinazolin-3-yl]-8-[7-[4-[(2,6-dioxo-3-piperidyl)amino]-2-fluoro-phenyl]-3-oxabicyclo[3.3.1]nonan-9-yl]-1-oxa-8-azaspiro[4.5]decane (Compound 194)

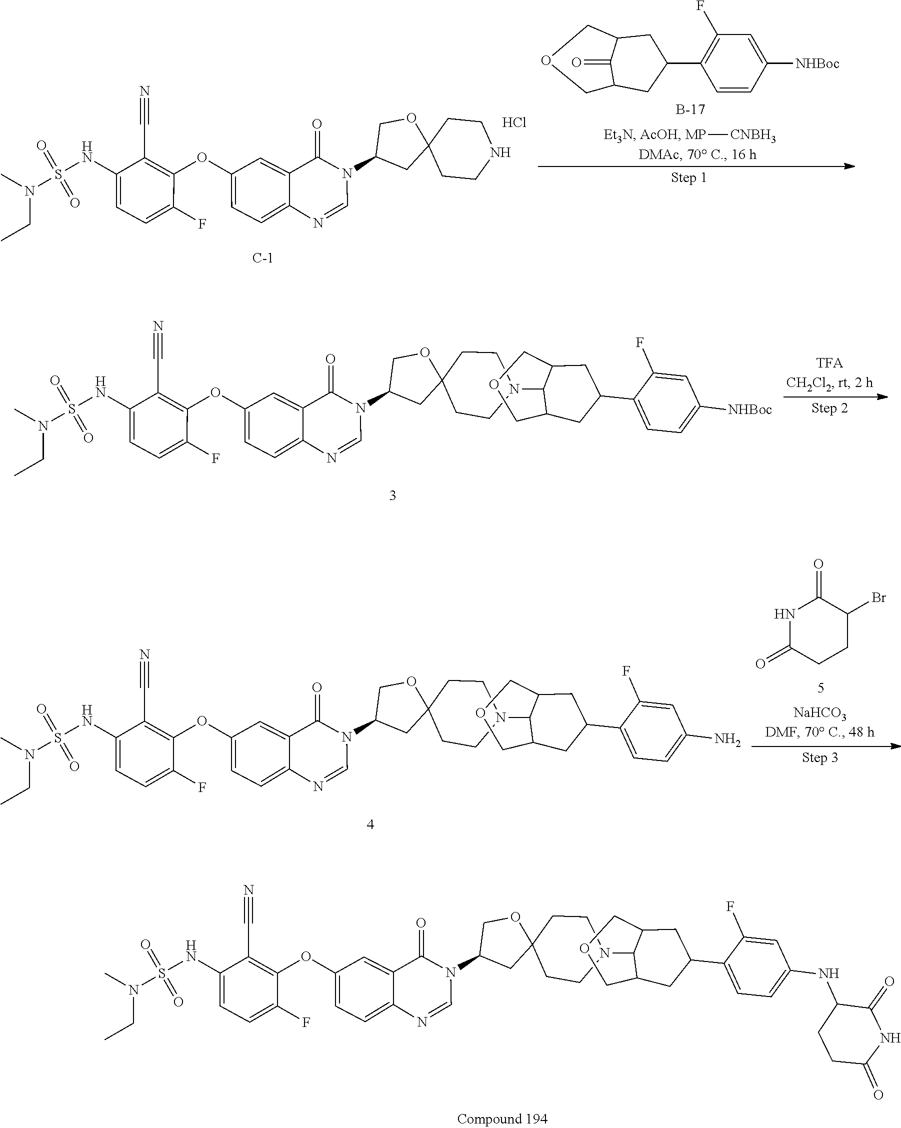

Into a 40 mL pressure released glass vial containing a well stirred solution of tert-butyl N-[3-fluoro-4-(9-oxo-3-oxabicyclo[3.3.1]nonan-7-yl)phenyl]carbamate (B-17) (170 mg, 486.55 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (C-1) (288.56 mg, 486.55 mol, HCl salt) in DMAc (4 mL) was added triethylamine (2.43 mmol, 339.08 μL) and stirred at room temperature for 10 minutes. Subsequently, AcOH (2.43 mmol, 139.13 μL) was added and stirred at room temperature for 2 hours. Finally, MP-CNBH₃ (300 mg) was added and stirred at 70° C. for 16 hours. After completion of the reaction, reaction mixture was diluted with dichloromethane (10 mL), filtered through cotton pad and filtrate was concentrated under reduced pressure to get crude which was purified by reverse phase by using RediSep R_{f} Gold® 100 g column and [Mobile phase A: 10 mM NH₄HCO₃ in milli-Q water; Mobile phase B: acetonitrile]. Collected fractions were lyophilized to afford tert-butyl N-[4-[9-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-3-oxabicyclo[3.3.1]nonan-7-yl]-3-fluoro-phenyl]carbamate (3, 150 mg, 166.85 mol, 34% yield) as an off-white solid. UPLC-MS (ES+): m/z 890.4 [M+H]⁺.

### Step 2

Into a 25 mL single-neck round-bottom flask containing a well stirred solution of tert-butyl N-[4-[9-[(3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decan-8-yl]-3-oxabicyclo[3.3.1]nonan-7-yl]-3-fluoro-phenyl]carbamate (3, 130 mg, 146.07 mol) in anhydrous dichloromethane (3 mL) was added TFA (730.33 mol, 55.89 μL) at room temperature. The resulting mixture was stirred at room temperature for 2 hours. After completion of the reaction, reaction mixture was concentrated under reduced pressure to get a crude which was triturated with methyl tert-butyl ether (10 mL) and dried under reduced pressure to afford (3R)-8-[7-(4-amino-2-fluoro-phenyl)-3-oxabicyclo[3.3.1]nonan-9-yl]-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (4, 120 mg, 107.53 mol, 74% yield, TFA salt) as an off-white solid. UPLC-MS (ES+): m/z 790.2 [M+H]⁺.

### Step 3

Into a 40 mL pressure glass vial containing a well stirred solution of (3R)-8-[7-(4-amino-2-fluoro-phenyl)-3-oxabicyclo[3.3.1]nonan-9-yl]-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (4, 120 mg, 132.76 mol, TFA salt) and 3-bromopiperidine-2,6-dione (5, 152.94 mg, 796.54 mol) in N,N-dimethylformamide (4 mL) was added NaHCO₃(55.76 mg, 663.78 mol) stirred at 70° C. for 48 hours. After completion of the reaction, reaction mixture was diluted with dichloromethane (20 mL), filtered through celite pad and filtrate was concentrated under reduced pressure to get crude which was purified by reverse phase by using RediSep R_{f} Gold® 100 g column and [Mobile phase A: 10 mM NH₄HCO₃ in milli-Q water; Mobile phase B: acetonitrile]. Eluted fractions were lyophilized to afford 40 mg compound with 44% purity (LC-MS44%). Further purified by Prep-HPLC by following method: [X-BRIDGE C18 (150 mm×19 mm×5 μm), Mobile phase A: 10 mM NH₄HCO₃ in milli-Q water & NH₄OH (pH-10); Mobile phase B: acetonitrile, flow rate 15 mL/min]. Eluted fractions were lyophilized to afford (3R)-3-[6-[2-cyano-3-[[ethyl(methyl)sulfamoyl]amino]-6-fluoro-phenoxy]-4-oxo-quinazolin-3-yl]-8-[7-[4-[(2,6-dioxo-3-piperidyl)amino]-2-fluoro-phenyl]-3-oxabicyclo[3.3.1]nonan-9-yl]-1-oxa-8-azaspiro[4.5]decane (Compound 194, 7 mg, 7.71 mol, 6% yield) as an off-white solid. UPLC-MS (ES+): m/z 901.3 [M+H]⁺. ¹H NMR (400 MHz, DMSO-d₆): δ 10.78 (s, 1H), 10.19 (s, 1H), 8.36 (s, 1H), 7.79 (d, J=8.80 Hz, 2H), 7.69 (dd, J=3.20, 9.00 Hz, 1H), 7.48-7.42 (m, 1H), 7.37 (d, J=2.80 Hz, 1H), 7.01 (t, J=8.40 Hz, 1H), 6.46 (d, J=8.40 Hz, 1H), 6.41-6.38 (m, 1H), 5.94 (d, J=7.60 Hz, 1H), 5.32-5.28 (m, 1H), 4.33-4.28 (m, 1H), 4.31-3.98 (m, 3H), 3.65-3.62 (m, 2H), 3.37-3.36 (m, 2H), 3.33-3.30 (m, 2H), 3.15-3.13 (m, 2H), 2.76-2.70 (m, 5H), 2.68-2.67 (m, 2H), 2.40-2.40 (m, 2H), 2.19-2.17 (m, 2H), 2.10-1.54 (m, 11H), 1.05 (t, J=7.20 Hz, 3H).

### Synthesis D109: Synthesis of 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-hydroxy-4-piperidyl]methoxy]quinoxaline (Compound 195 and Compound 196)

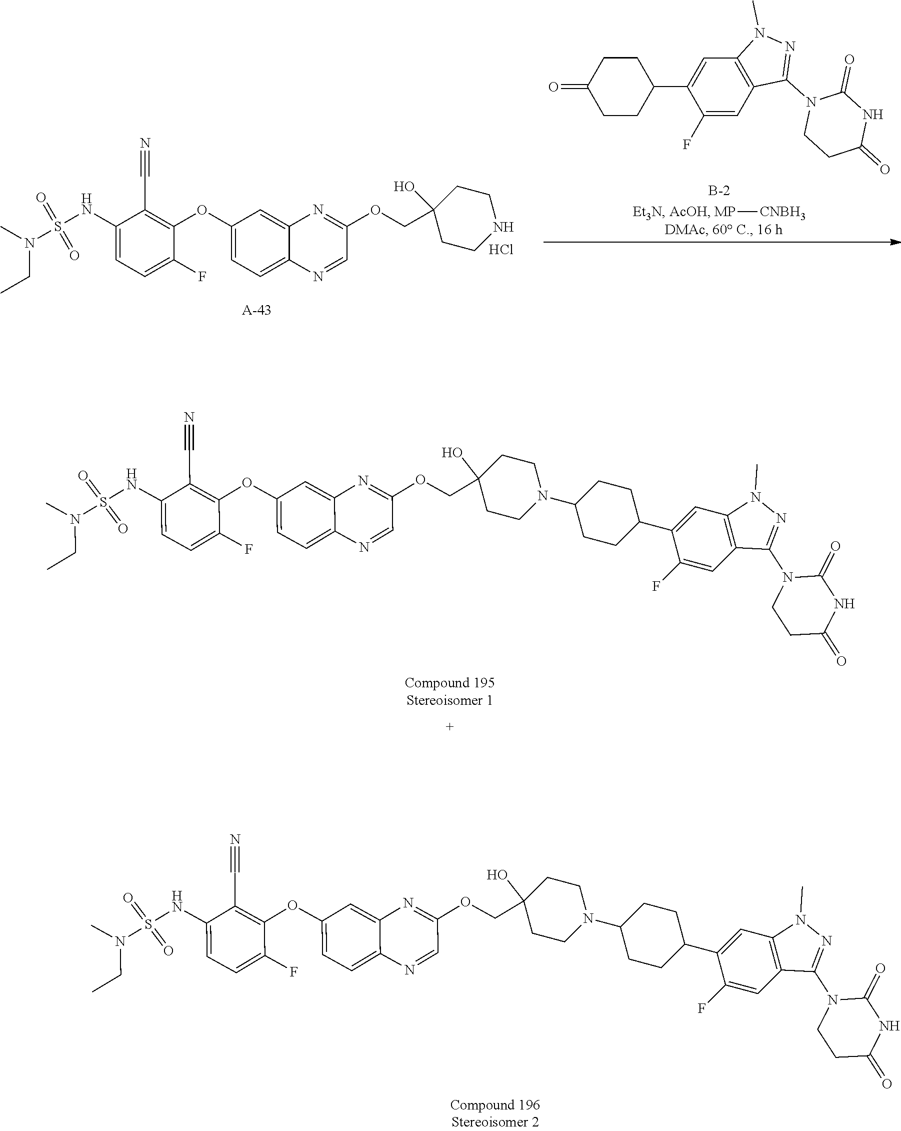

Into a 20 mL glass vial with pressure released cap containing a well-stirred solution of 7-[2-cyano-3-[[ethyl(methyl)sulfamoyl]amino]-6-fluoro-phenoxy]-2-[(4-hydroxy-4-piperidyl)methoxy]quinoxaline (A-43) (350 mg, 604.90 mol, HCl salt) in DMAc (10 mL) was added triethylamine (1.81 mmol, 252.94 μL) at room temperature, reaction mixture was stirred for 5 minutes. Subsequently, AcOH (1.21 mmol, 69.19 μL) and 1-[5-fluoro-1-methyl-6-(4-oxocyclohexyl)indazol-3-yl]hexahydropyrimidine-2,4-dione (B-2) (325.17 mg, 907.36 mol) were added at room temperature and stirred for 2 hours at room temperature. Finally, MP-CNBH₃ (350 mg, 604.90 mol) was added at room temperature. Resulting reaction mixture was stirred at 60° C. for 16 hours. After completion of the reaction, the reaction mixture was filtered through cotton pad and filtrate was concentrated under reduced pressure to get a crude residue, which was purified by prep HPLC by using a method Column: X-Bridge C18 (150 mm×19 mm×5 μm) and [Mobile phase A: 10 mm NH₄HCO₃ and (pH-10 with NH₄OH) in milli-Q water; Mobile phase B: acetonitrile]. First eluted fraction (Peak-1) was lyophilized to afford Compound 195 (assigned as stereoisomer 1).

Peak-1: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-hydroxy-4-piperidyl]methoxy]quinoxaline (Compound 195, 55 mg, 62.73 mol, 10% yield) as an off white-solid. UPLC-MS (ES+): m/z 873.3 [M+H]⁺. ¹H NMR (400 MHz, DMSO-d₆): δ 10.56 (s, 1H), 8.99 (bs, 1H), 8.53 (s, 1H), 8.07 (d, J=9.20 Hz, 1H), 7.56-7.23 (m, 5H), 6.93 (bs, 1H), 5.2-5.3 (m, 1H), 4.35-4.45 (m, 2H), 4.03 (s, 3H), 3.91 (t, J=6.80 Hz, 2H), 3.37-3.32 (m, 1H), 3.32-3.01 (m, 6H), 2.76 (t, J=6.80 Hz, 2H), 2.68-2.60 (m, 3H), 2.15-2.22 (m, 2H), 2.10-1.60 (m, 11H), 1.04 (t, J=7.20 Hz, 3H).

Similarly, the second eluted fraction (Peak-2) was lyophilized to afford Compound 196 (assigned as stereoisomer 2).

Peak-2: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-hydroxy-4-piperidyl]methoxy]quinoxaline (Compound 196, 100 mg, 110.59 mol, 18% yield) as a white-solid. UPLC-MS (ES+): m/z 873.3 [M+H]⁺. ¹H NMR (400 MHz, DMSO-d₆): δ 10.56 (s, 1H), 8.53-8.66 (m, 2H), 8.06 (d, J=9.20 Hz, 1H), 7.51-7.28 (m, 5H), 6.92 (bs, 1H), 5.2-5.3 (m, 1H), 4.35-4.45 (m, 2H), 4.03 (s, 3H), 3.91 (t, J=3.20 Hz, 2H), 3.60-3.40 (m, 2H), 3.20-2.90 (m, 5H), 2.76 (t, J=6.80 Hz, 2H), 2.60-2.50 (m, 3H), 2.20-1.65 (m, 13H), 1.03 (t, J=7.20 Hz, 3H).

### Synthesis D110: Synthesis of 6-[2,6-difluoro-3-[[2-hydroxyethyl(methyl)sulfamoyl]amino]phenoxy]-3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazoline (Compound 197)

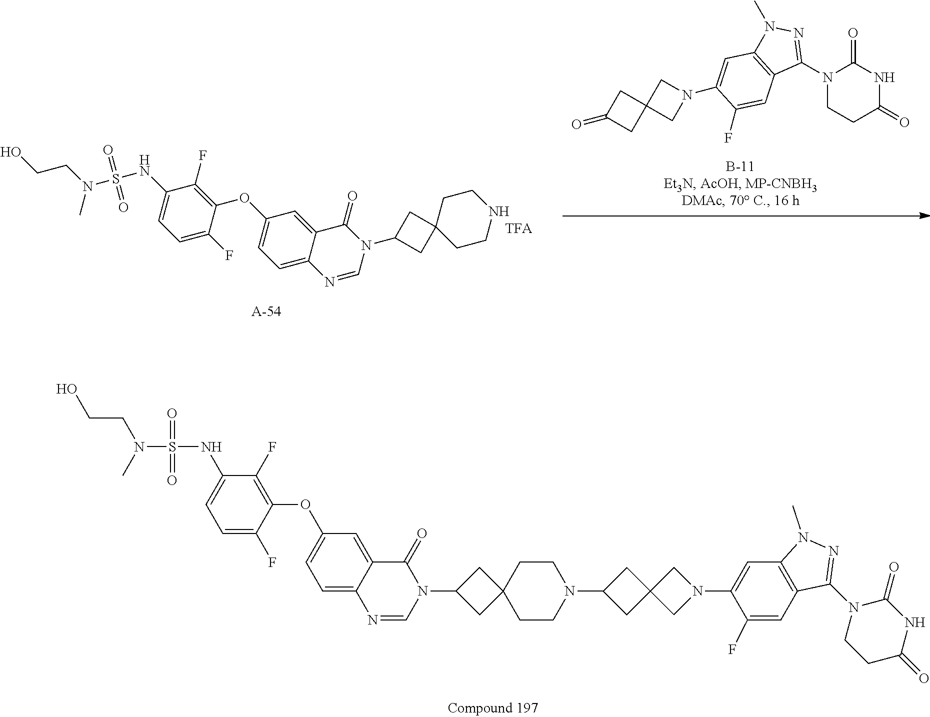 [SMILES: Cn1nc(N2CCC(=O)NC2=O)c2cc(F)c(N3CC4(CC(=O)C4)C3)cc21 ; CN(CCO)S(=O)(=O)Nc1ccc(F)c(Oc2ccc3ncn(C4CC5(CCNCC5)C4)c(=O)c3c2)c1F ; "B-11Et3N, AcOH, MP-CNBH3DMAc, 70° C., 16 h" ; "A-54" ; CN(CCO)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1F ; "TFA"]

Into a 8 mL pressure vial containing a well stirred solution of 3-(7-azaspiro[3.5]nonan-2-yl)-6-[2,6-difluoro-3-[[2-hydroxyethyl(methyl)sulfamoyl]amino]phenoxy]-4-oxo-quinazoline (A-54) (100 mg, 150.69 mol, TFA salt) in DMAc (2 mL) was added triethylamine (150.69 mol, 21.00 μL) and stirred at room temperature for 30 min. Subsequently, AcOH (452.07 mol, 25.85 μL) and 1-[5-fluoro-1-methyl-6-(6-oxo-2-azaspiro[3.3]heptan-2-yl)indazol-3-yl]hexahydropyrimidine-2,4-dione (B-11) (61.56 mg, 165.76 mol) were added and the reaction mixture was stirred at room temperature for 4 hours. Finally, MP-CNBH₃ (100 mg, 150.69 mol) was added, and the reaction mixture was stirred at 70° C. for 16 hours. After completion of the reaction, the reaction mixture was filtered through a cotton pad and the filtrate was concentrated under reduced pressure to obtain a crude residue which was purified by prep HPLC [Column: X-BRIDGE C8 (250 mm×19 mm×5 μm); Mobile phase A: 10 mM NH₄HCO₃ in milli-Q water; Mobile phase B: acetonitrile]. Collected fractions were lyophilized to afford 6-[2,6-difluoro-3-[[2-hydroxyethyl(methyl)sulfamoyl]amino]phenoxy]-3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazoline (Compound 197, 26.5 mg, 29.20 mol, 19% yield) as a white solid. UPLC-MS (ES+): m/z 905.5 [M+H]⁺. ¹H NMR (400 MHz, DMSO-d₆): δ 10.50 (s, 1H), 9.73 (bs, 1H), 8.39 (s, 1H), 7.77 (d, J=8.80 Hz, 1H), 7.65 (dd, J=3.20, 9.00 Hz, 1H), 7.47-7.41 (m, 1H), 7.38-7.34 (m, 2H), 7.26 (d, J=12.80 Hz, 1H), 6.49 (d, J=7.60 Hz, 1H), 4.97-4.88 (m, 1H), 4.87-4.54 (m, 1H), 3.99 (s, 2H), 3.89-3.86 (m, 7H), 3.47 (t, J=6.00 Hz, 2H), 3.11 (t, J=6.00 Hz, 2H), 2.74 (s, 3H), 2.71 (t, J=6.80 Hz, 2H), 2.33-2.13 (m, 9H), 2.08-1.96 (m, 3H), 1.71-1.54 (m, 4H).

### Synthesis D111: Synthesis of 6-[2-Cyano-3-[[ethyl(methyl)sulfamoyl]amino]-6-fluoro-phenoxy]-3-[7-[7-[3-(2,4-dioxohexahydropyrimidin-1-yl)-5-fluoro-1-methyl-indazol-6-yl]-7-azaspiro[3.5]nonan-2-yl]-7-azaspiro[3.5]nonan-2-yl]-4-oxo-quinazoline (Compound 198)

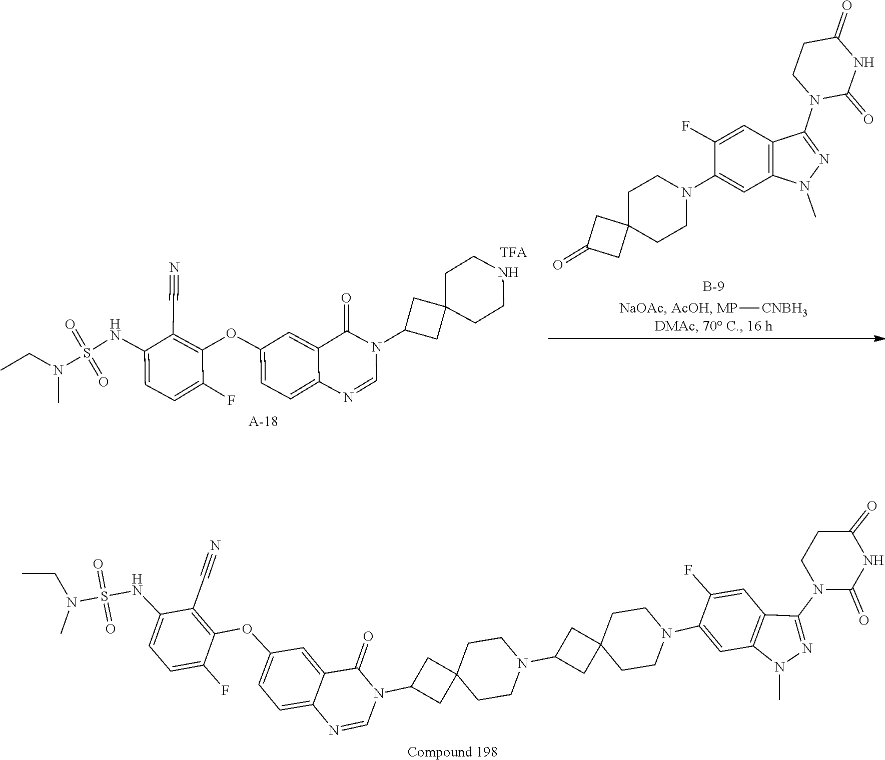

Into a 40 mL pressure vial containing a well-stirred solution of 1-[5-fluoro-1-methyl-6-(2-oxo-7-azaspiro[3.5]nonan-7-yl)indazol-3-yl]hexahydropyrimidine-2,4-dione (B-9) (700 mg, 1.51 mmol), 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (986.66 mg, 1.51 mmol, TFA salt) in DMAc (15 mL) was added anhydrous NaOAc (370.92 mg, 4.52 mmol) at room temperature under nitrogen atmosphere and stirred for 5 minutes. After 5 minutes, AcOH (4.52 mmol, 258.6 μL) was added and stirred for 2 hours at room temperature. Subsequently, MP-CNBH₃ (700 mg, 1.51 mmol) was added and stirred at 70° C. for 16 hours. After completion of the reaction, reaction mixture was filtered through cotton pad and filtrate was concentrated under reduced pressure to get a crude residue which was purified by preparative HPLC [Column: X-Bridge C18 (250 mm*19 mm*5 m), Mobile phase A: 10 mM NH₄HCO₃ (pH was adjusted to 10 with NH₄OH) in milli-Q water; Mobile phase B: acetonitrile]. Fractions were collected and lyophilized to afford 6-[2-cyano-3-[[ethyl(methyl)sulfamoyl]amino]-6-fluoro-phenoxy]-3-[7-[7-[3-(2,4-dioxohexahydropyrimidin-1-yl)-5-fluoro-1-methyl-indazol-6-yl]-7-azaspiro[3.5]nonan-2-yl]-7-azaspiro[3.5]nonan-2-yl]-4-oxo-quinazoline (Compound 198, 485 mg, 518.95 mol, 34% yield) as an off-white solid. UPLC-MS (ES+): m/z 924.3 [M+H]⁺. ¹H NMR (400 MHz, DMSO-d₆): δ 10.53 (s, 1H), 9.25 (bs, 1H), 8.38 (s, 1H), 7.76 (d, J=9.20 Hz, 1H), 7.62 (dd, J=2.80, 8.80 Hz, 1H), 7.41-7.28 (m, 4H), 7.11 (d, J=6.80 Hz, 1H), 5.00-4.91 (m, 1H), 3.94 (s, 3H), 3.89 (t, J=6.40 Hz, 2H), 3.05-2.95 (m, 7H), 2.74 (t, J=6.80 Hz, 2H), 2.59 (s, 3H), 2.45-2.35 (m, 3H), 2.21-2.11 (m, 2H), 2.00-1.68 (m, 9H), 1.03 (t, J=7.20 Hz, 3H).

### Synthesis D112: Synthesis of Synthesis of 3-[7-[4-[4-[(2,6-dioxo-3-piperidyl)amino]-2-fluoro-phenyl]cyclohexyl]-7-azaspiro[3.5]nonan-2-yl]-6-[3-[[ethyl(methyl)sulfamoyl]amino]-2,6-difluoro-benzoyl]-4-oxo-quinazoline (Compound 199 and Compound 200)

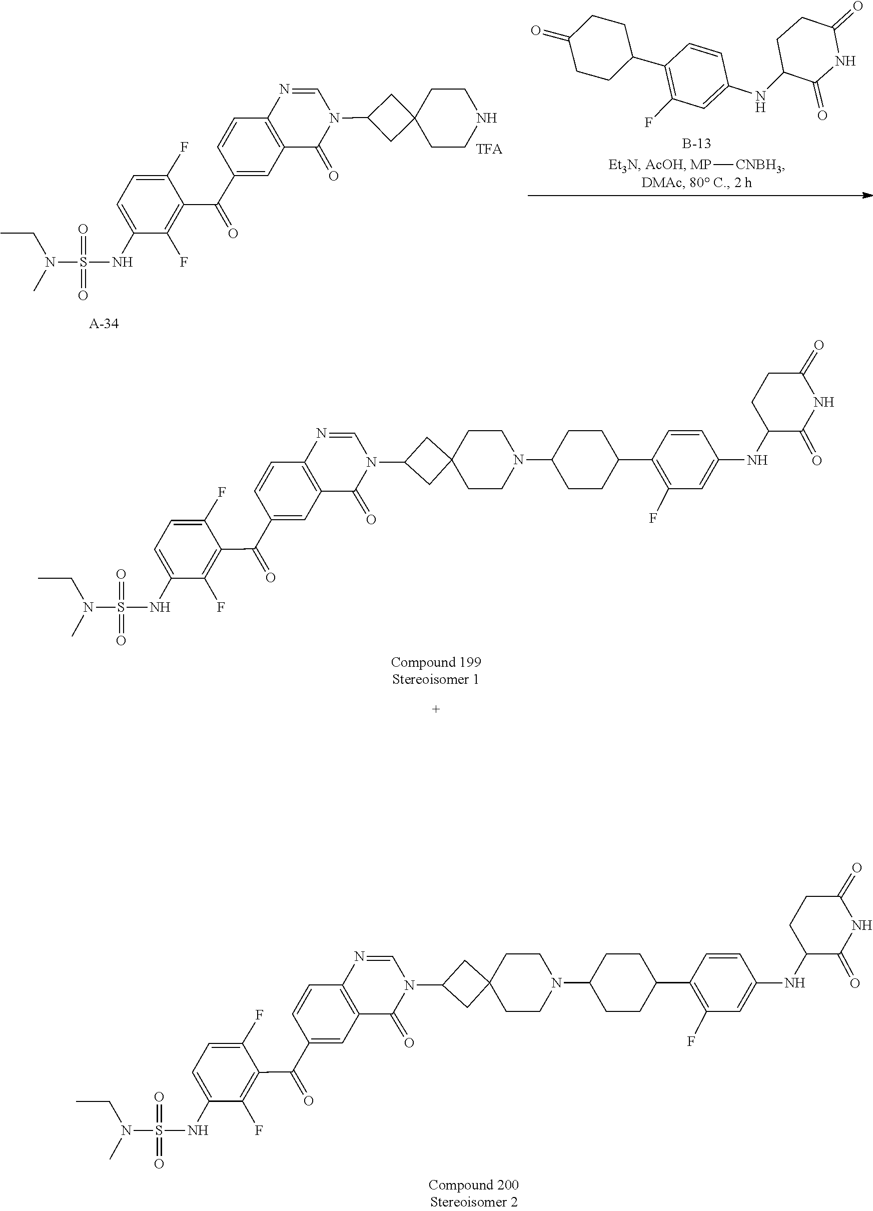

Into a 20 mL pressure vial containing a well-stirred solution of 3-(7-azaspiro[3.5]nonan-2-yl)-6-[3-[[ethyl(methyl)sulfamoyl]amino]-2,6-difluoro-benzoyl]-4-oxo-quinazoline (A-34) (180.00 mg, 272.88 mol, TFA salt) in DMAc (4 mL) were added triethylamine (818.65 mol, 114.10 μL), 3-[3-fluoro-4-(4-oxocyclohexyl)anilino]piperidine-2,6-dione (B-13) (130.31 mg, 409.32 mol) and AcOH (648 mol, 33.56 μL) at room temperature under nitrogen atmosphere and the reaction mixture was stirred for 2 hours. Subsequently, MP-CNBH₃ (180 mg, 272.88 mol) was added at room temperature. The resulting reaction mixture was stirred at 80° C. for 2 hours. After completion of the reaction, reaction mixture was filtered through cotton and concentrated under reduced pressure to get a crude residue, which was purified by reverse phase column chromatography (Column: Redisep R_{f} Gold® reversed-phase C18-teledyne ISCO, 120 g, mobile phase A: 10 mM NH₄HCO₃ in milli-Q water; mobile phase B: acetonitrile). The obtained fractions were lyophilized to get a mixture of cis and trans isomers, which further purified by preparative HPLC using the method [Column: X-Bridge C18 (150 mm×19 mm×5 μm); Mobile phase A: 10 mM NH₄HCO₃ in milli-Q water; Mobile phase B: 100% acetonitrile]. The first eluting fraction was concentrated under reduced pressure at 40° C. and lyophilized to afford Compound 199 (assigned as stereoisomer 1).

Peak-1: 3-[7-[4-[4-[(2,6-dioxo-3-piperidyl)amino]-2-fluoro-phenyl]cyclohexyl]-7-azaspiro[3.5]nonan-2-yl]-6-[3-[[ethyl(methyl)sulfamoyl]amino]-2,6-difluoro-benzoyl]-4-oxo-quinazoline (Compound 199, 23 mg, 25.89 mol, 9% yield) as an off-white solid. UPLC-MS (ES+): m/z 848.2 [M+H]⁺. ¹H NMR (400 MHz, DMSO-d₆): δ 10.79 (s, 1H), 9.75 (bs, 1H), 8.58 (s, 1H), 8.38 (d, J=1.60 Hz, 1H), 8.28 (dd, J=2.00, 8.60 Hz, 1H), 7.87 (d, J=8.80 Hz, 1H), 7.71-7.65 (m, 1H), 7.32 (t, J=8.40 Hz, 1H), 6.98 (t, J=4.40 Hz, 1H), 6.45-6.41 (m, 2H), 5.98 (d, J=8.00 Hz, 1H), 4.95-4.86 (m, 1H), 4.33-4.27 (m, 1H), 3.10 (q, J=7.20 Hz, 2H), 2.71-2.67 (m, 4H), 2.58-2.53 (m, 5H), 2.43-2.33 (m, 4H), 2.27-2.21 (m, 2H), 2.11-2.05 (m, 1H), 1.63-1.63 (m, 9H), 1.47-1.38 (m, 4H), 1.02 (t, J=7.20 Hz, 3H).

Similarly, the second eluted fraction was concentrated under reduced pressure at 40° C. and lyophilized to afford Compound 200 (assigned as stereoisomer 2).

Peak-2: 3-[7-[4-[4-[(2,6-dioxo-3-piperidyl)amino]-2-fluoro-phenyl]cyclohexyl]-7-azaspiro[3.5]nonan-2-yl]-6-[3-[[ethyl(methyl)sulfamoyl]amino]-2,6-difluoro-benzoyl]-4-oxo-quinazoline (Compound 200, 31 mg, 35.11 mol, 13% yield) as a pale yellow solid. UPLC-MS (ES+): m/z 848.2 [M+H]⁺. ¹H NMR (400 MHz, DMSO-d₆): δ 10.79 (s, 1H), 9.75 (bs, 1H), 8.59 (s, 1H), 8.38 (d, J=1.60 Hz, 1H), 8.28 (dd, J=2.40, 8.60 Hz, 1H), 7.87 (d, J=8.80 Hz, 1H), 7.72-7.66 (m, 1H), 7.34 (t, J=8.80 Hz, 1H), 6.98 (t, J=8.80 Hz, 1H), 6.48-6.41 (m, 2H), 5.97 (d, J=7.60 Hz, 1H), 4.93-4.86 (m, 1H), 4.33-4.27 (m, 1H), 3.11 (q, J=7.20 Hz, 2H), 2.78-2.67 (m, 5H), 2.61-2.55 (m, 2H), 2.42-2.33 (m, 4H), 2.28-2.23 (m, 3H), 2.11-2.07 (m, 1H), 2.01-1.98 (m, 2H), 1.88-1.67 (m, 8H), 1.51-1.42 (m, 4H), 1.02 (t, J=7.20 Hz, 3H).

### Synthesis D113: Synthesis of 6-[2-cyano-3-[[ethyl(methyl)sulfamoyl]amino]-6-fluoro-phenoxy]-3-[[1-[4-[3-(2,4-dioxohexahydropyrimidin-1-yl)-5-fluoro-1-methyl-indazol-6-yl]cyclohexyl]-4-fluoro-4-piperidyl]methyl]-4-oxo-quinazoline (Compound 201 and Compound 202)

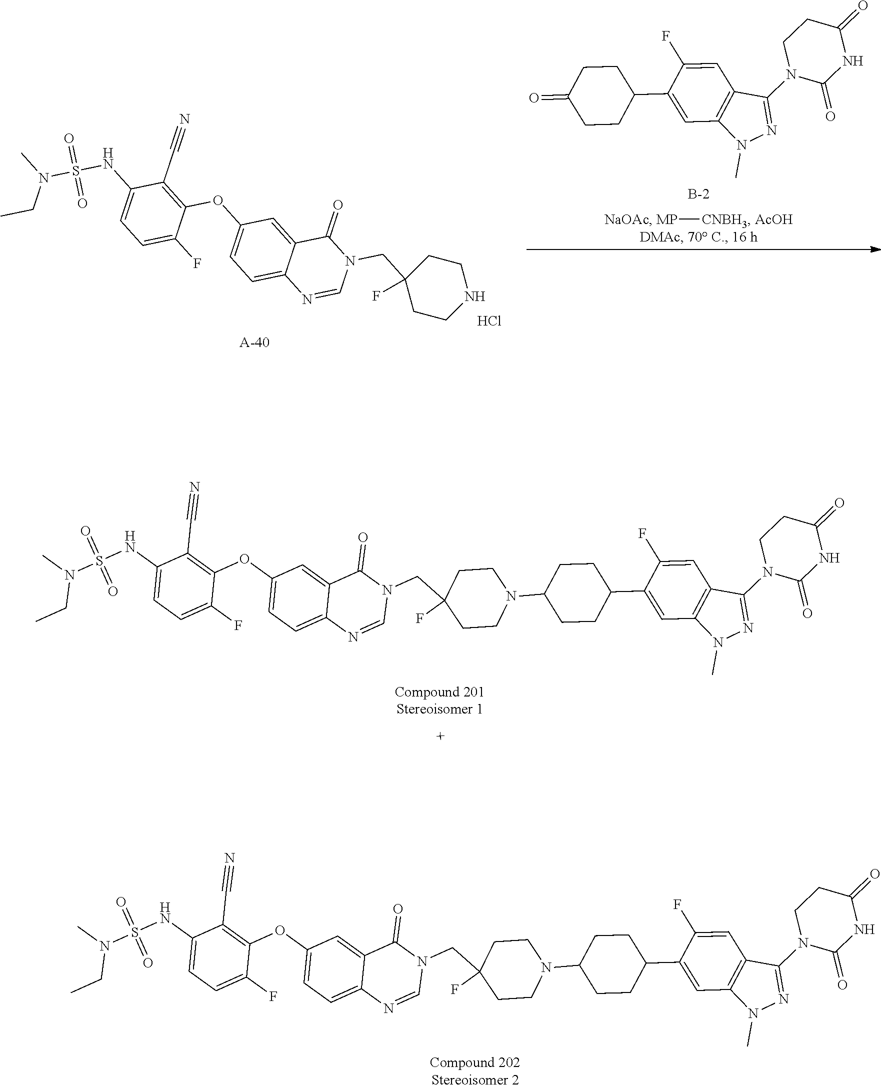

Into a 30 mL pressure vial containing a well-stirred solution of 6-[2-cyano-3-[[ethyl(methyl)sulfamoyl]amino]-6-fluoro-phenoxy]-3-[(4-fluoro-4-piperidyl)methyl]-4-oxo-quinazoline (A-40) (0.900 g, 1.58 mmol, HCl salt) and 1-[5-fluoro-1-methyl-6-(4-oxocyclohexyl)indazol-3-yl]hexahydropyrimidine-2,4-dione (B-2) (680.18 mg, 1.90 mmol) in DMAc (10 mL) was added NaOAc (648.72 mg, 7.91 mmol) and resulting mixture was stirred at room temperature for 1 hour. After 1 h, AcOH (4.74 mmol, 271.37 μL) was added and stirred at room temperature for 2 hours. Subsequently, MP-CNBH₃ (1.2 g, 1.58 mmol) was added and resulting mixture was stirred at 70° C. for 16 hours. After completion of the reaction, reaction mixture was filtered through sintered funnel and the filtrate was concentrated under reduced pressure to get crude residue which was purified by preparative HPLC [Column: X-Bridge C18 (150 mm*19 mm*5 μm), Mobile phase A:10 mM NH₄HCO₃ (pH was adjusted to 10 with NH₄OH), Mobile phase B: 100% acetonitrile]. The first eluting fraction (Peak-1) was collected and lyophilized to afford Compound 201 (assigned as stereoisomer 1).

Peak-1: 6-[2-cyano-3-[[ethyl(methyl)sulfamoyl]amino]-6-fluoro-phenoxy]-3-[[1-[4-[3-(2,4-dioxohexahydropyrimidin-1-yl)-5-fluoro-1-methyl-indazol-6-yl]cyclohexyl]-4-fluoro-4-piperidyl]methyl]-4-oxo-quinazoline (Compound 201, 0.065 g, 71.91 mol, 5% yield) as a white solid. UPLC-MS (ES+): m/z 875.3 [M+H]⁺. ¹H NMR (400 MHz, DMSO-d₆): δ 10.55 (s, 1H), 9.53 (bs, 1H), 8.23 (s, 1H), 7.80 (d, J=9.20 Hz, 1H), 7.69 (dd, J=3.20, 9.00 Hz, 1H), 7.65-7.50 (m, 2H), 7.39-7.33 (m, 3H), 4.30-4.30 (m, 2H), 4.00 (s, 3H), 3.90 (t, J=6.80 Hz, 2H), 3.25-2.72 (m, 10H), 2.64 (s, 3H), 2.12-1.85 (m, 8H), 1.70-1.55 (m, 4H), 1.04 (t, J=7.20 Hz, 3H).

Similarly, second eluting fraction (Peak-2) was lyophilized to afford Compound 202 (assigned as stereoisomer 2).

Peak-2: 6-[2-cyano-3-[[ethyl(methyl)sulfamoyl]amino]-6-fluoro-phenoxy]-3-[[1-[4-[3-(2,4-dioxohexahydropyrimidin-1-yl)-5-fluoro-1-methyl-indazol-6-yl]cyclohexyl]-4-fluoro-4-piperidyl]methyl]-4-oxo-quinazoline (Compound 202, 0.044 g, 50.24 mol, 3% yield) as off-white solid. UPLC-MS (ES+): m/z 875.3 [M+H]⁺. ¹H NMR (400 MHz, DMSO-d₆): δ 10.56 (s, 1H), 8.23 (s, 1H), 7.80 (d, J=8.80 Hz, 1H), 7.70-7.51 (m, 2H), 7.47 (d, J=6.00 Hz, 1H), 7.39-7.36 (m, 3H), 4.40-4.28 (m, 2H), 3.93 (s, 3H), 3.90 (t, J=6.80 Hz, 2H), 3.18-2.86 (m, 5H), 2.76 (t, J=6.80 Hz, 2H), 2.68-2.66 (m, 3H), 2.20-1.50 (m, 13H), 1.04 (t, J=7.20 Hz, 3H).

### Synthesis D114: Synthesis of 6-[2-cyano-3-[[ethyl(methyl)sulfamoyl]amino]-6-fluoro-phenoxy]-3-[2-[4-[3-(2,4-dioxohexahydropyrimidin-1-yl)-5-fluoro-1-methyl-indazol-6-yl]cyclohexyl]-2-azaspiro[3.3]heptan-6-yl]-4-oxo-quinazoline (Compound 203 and Compound 204)

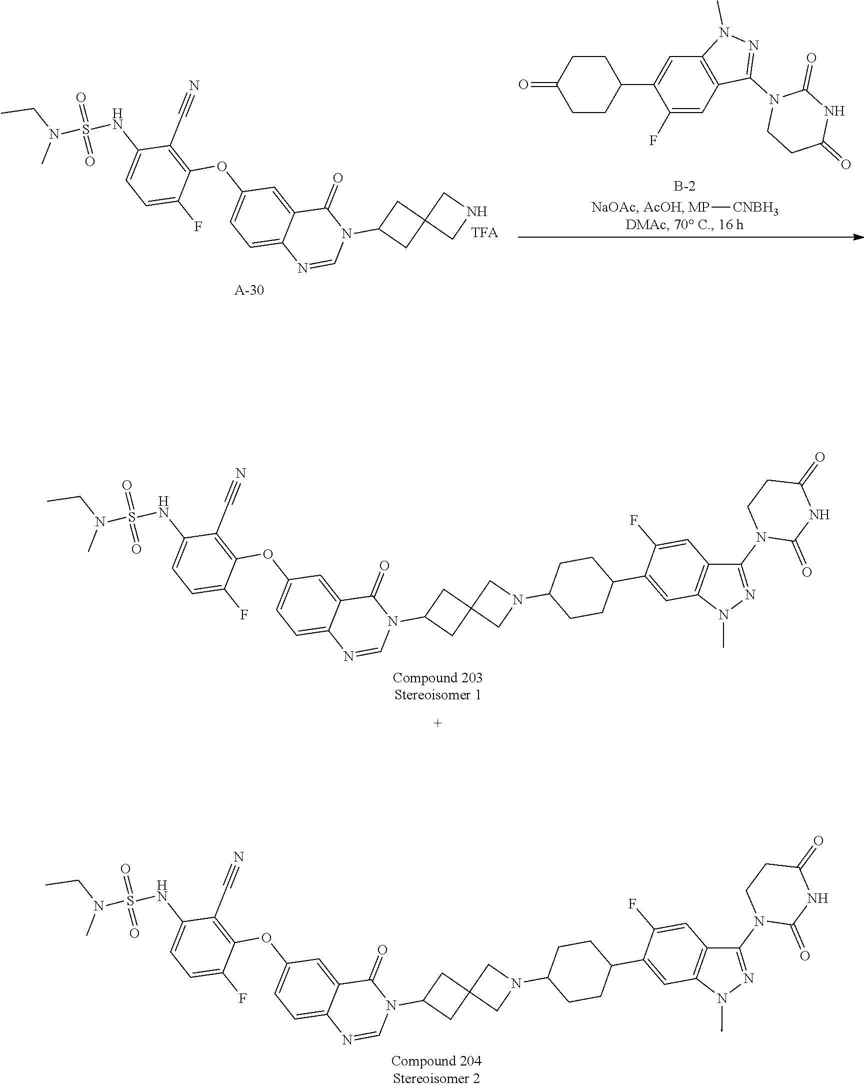

Into a 40 mL pressure vial containing a well-stirred solution of 3-(2-azaspiro[3.3]heptan-6-yl)-6-[2-cyano-3-[[ethyl(methyl)sulfamoyl]amino]-6-fluoro-phenoxy]-4-oxo-quinazoline (A-30) (400.00 mg, 638.39 mol, TFA salt) in DMAc (8 mL) was added NaOAc (157.11 mg, 1.92 mmol) followed by AcOH (1.28 mmol, 73.01 μL) at room temperature and stirred for 15 minutes. Subsequently, 1-[5-fluoro-1-methyl-6-(4-oxocyclohexyl)indazol-3-yl]hexahydropyrimidine-2,4-dione (B-2) (316.39 mg, 829.90 mol) was added and stirred at room temperature for 1 hour. Finally, MP-CNBH₃ (400 mg, 638.39 mol) was added, and the reaction mixture was stirred at 70° C. for 16 hours. After completion of the reaction, reaction mixture was diluted with dichloromethane (10 mL), filtered and filtrate was concentrated under reduced pressure to get a crude residue which was purified by preparative HPLC [Column: X-Bridge C18 (250 mm*19 mm*5 μm), Mobile phase A: 10 mM NH₄HCO₃ & NH₄OH (pH 10) in milli-Q water; Mobile phase B: acetonitrile]. The first eluting fraction (Peak-1) was lyophilized to afford Compound 203 (assigned as stereoisomer 1).

Peak-1: 6-[2-cyano-3-[[ethyl(methyl)sulfamoyl]amino]-6-fluoro-phenoxy]-3-[2-[4-[3-(2,4-dioxohexahydropyrimidin-1-yl)-5-fluoro-1-methyl-indazol-6-yl]cyclohexyl]-2-azaspiro[3.3]heptan-6-yl]-4-oxo-quinazoline (Compound 203, 27 mg, 30.37 mol, 5% yield) as an off-white solid. UPLC-MS (ES+): m/z 855.3 [M+H]⁺. ¹H NMR (400 MHz, DMSO-d₆): δ 10.56 (s, 1H), 9.85 (bs, 1H), 8.35 (s, 1H), 7.77 (d, J=8.80 Hz, 1H), 7.65 (dd, J=3.20, 9.00 Hz, 1H), 7.53 (d, J=5.60 Hz, 1H), 7.45-7.15 (m, 4H), 4.87-4.80 (m, 1H), 4.35-4.05 (m, 4H), 4.00 (s, 3H), 3.91 (t, J=6.80 Hz, 2H), 3.05-2.95 (m, 2H), 2.91-2.72 (m, 7H), 2.59 (s, 3H), 2.10-1.94 (m, 4H), 1.84-1.55 (m, 3H), 1.41-1.21 (m, 2H), 1.03 (t, J=7.20 Hz, 3H).

Similarly, the second eluted fraction (Peak-2) was lyophilized to afford Compound 204 (assigned as stereoisomer 2).

Peak-2: 6-[2-cyano-3-[[ethyl(methyl)sulfamoyl]amino]-6-fluoro-phenoxy]-3-[2-[4-[3-(2,4-dioxohexahydropyrimidin-1-yl)-5-fluoro-1-methyl-indazol-6-yl]cyclohexyl]-2-azaspiro[3.3]heptan-6-yl]-4-oxo-quinazoline (Compound 204, 25 mg, 28.65 mol, 4% yield) as an off-white solid. UPLC-MS (ES+): m/z 855.3 [M+H]⁺. ¹H NMR (400 MHz, DMSO-d₆): δ 10.56 (s, 1H), 9.48 (bs, 1H), 8.36 (s, 1H), 7.76 (d, J=9.20 Hz, 1H), 7.65 (dd, J=2.40, 8.80 Hz, 1H), 7.52-7.35 (m, 3H), 7.31-7.23 (m, 2H), 4.87-4.79 (m, 1H), 4.45-4.18 (m, 3H), 4.01 (s, 3H), 3.91 (t, J=6.80 Hz, 2H), 3.60-3.51 (m, 1H), 3.10-2.85 (m, 5H), 2.76 (t, J=6.80 Hz, 2H), 2.73-2.68 (m, 2H), 2.59 (s, 3H), 1.98-1.55 (m, 9H), 1.03 (t, J=7.20 Hz, 3H).

### Synthesis D115: Synthesis of 6-[2-cyano-3-[[ethyl(methyl)sulfamoyl]amino]-6-fluoro-phenoxy]-3-[6-[4-[3-(2,4-dioxohexahydropyrimidin-1-yl)-5-fluoro-1-methyl-indazol-6-yl]cyclohexyl]-6-azaspiro[3.4]octan-2-yl]-4-oxo-quinazoline (Compound 205 and Compound 206)

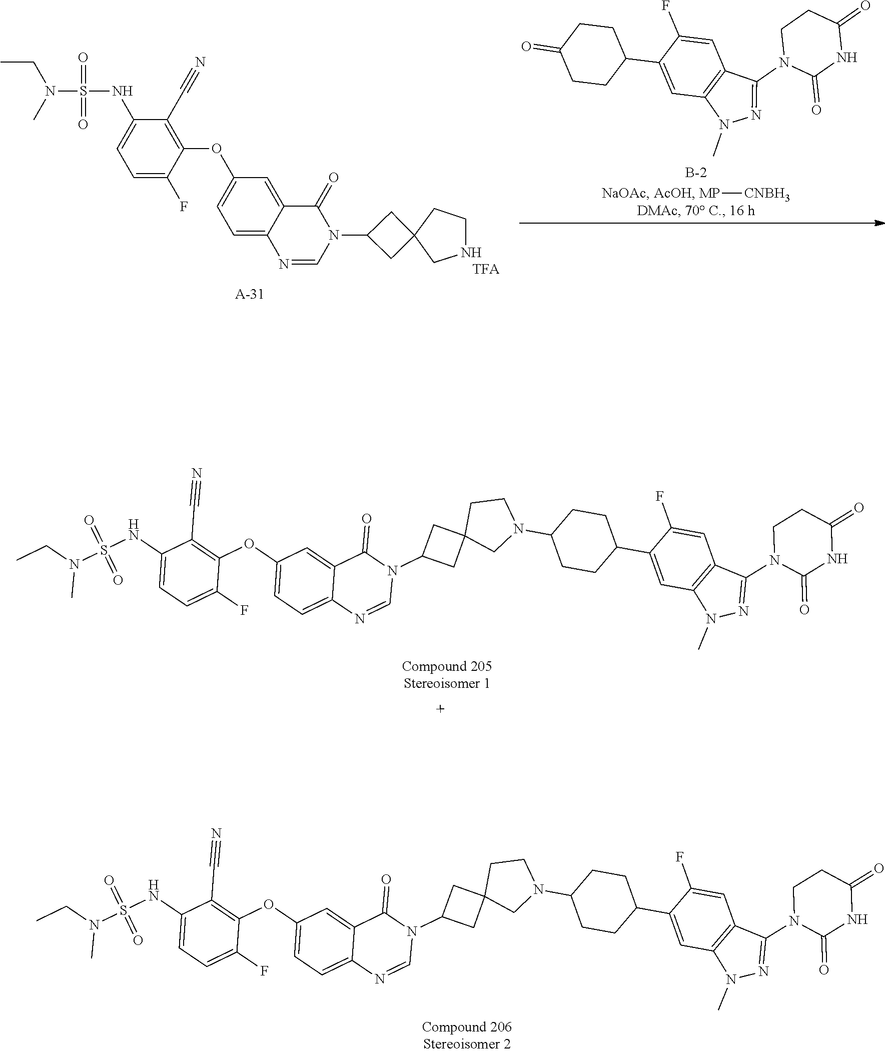

Into a 40 mL pressure vial containing a well-stirred solution of 3-(6-azaspiro[3.4]octan-2-yl)-6-[2-cyano-3-[[ethyl(methyl)sulfamoyl]amino]-6-fluoro-phenoxy]-4-oxo-quinazoline (A-31) (380 mg, 522.01 mol, TFA salt) in DMAc (8.0 mL) was added NaOAc (214.11 mg, 2.61 mmol) followed by AcOH (1.57 mmol, 89.56 μL) at room temperature and stirred for 15 minutes. Subsequently, 1-[5-fluoro-1-methyl-6-(4-oxocyclohexyl)indazol-3-yl]hexahydropyrimidine-2,4-dione (B-2) (258.71 mg, 678.61 mol) was added and stirred at room temperature for 1 hour. Finally, MP-CNBH₃ (380 mg, 522.01 mol) was added, and the reaction mixture was stirred at 70° C. for 16 hours. After completion of the reaction, reaction mixture was diluted with dichloromethane (10 mL), filtered and filtrate was concentrated under reduced pressure to get a crude residue which was purified by preparative HPLC [Column: X-Bridge C8 (250 mm*19 mm*5 μm), Mobile phase A: 10 mM NH₄HCO₃ & NH₄OH (pH_10) in milli-Q water; Mobile phase B: acetonitrile]. The first eluting fraction (Peak-1) was lyophilized to afford Compound 205 (assigned as stereoisomer 1).

Peak-1: 6-[2-cyano-3-[[ethyl(methyl)sulfamoyl]amino]-6-fluoro-phenoxy]-3-[6-[4-[3-(2,4-dioxohexahydropyrimidin-1-yl)-5-fluoro-1-methyl-indazol-6-yl]cyclohexyl]-6-azaspiro[3.4]octan-2-yl]-4-oxo-quinazoline (Compound 205, 13 mg, 14.72 mol, 3% yield) as an off-white solid. UPLC-MS (ES+): m/z 869.3 [M+H]⁺. ¹H NMR (400 MHz, DMSO-d₆): δ 10.56 (s, 1H), 8.36 (s, 1H), 7.75 (d, J=9.20 Hz, 1H), 7.60 (dd, J=3.20, 9.00 Hz, 1H), 7.55 (d, J=6.00 Hz, 1H), 7.38-7.27 (m, 3H), 7.24-7.20 (m, 1H), 7.18-6.90 (m, 1H), 4.89-4.83 (m, 1H), 3.99 (s, 3H), 3.90 (t, J=6.80 Hz, 3H), 3.08-2.91 (m, 2H), 2.88-2.82 (m, 3H), 2.75 (t, J=6.80 Hz, 3H), 2.57-2.52 (m, 6H), 2.44-2.42 (m, 1H), 2.16-2.08 (m, 2H), 1.96-1.92 (m, 4H), 1.66-1.60 (m, 3H), 1.46-1.38 (m, 2H), 1.02 (t, J=7.20 Hz, 3H).

Similarly, the second eluted fraction (Peak-2) was lyophilized to afford Compound 206 (assigned as stereoisomer 2).

Peak-2: 6-[2-cyano-3-[[ethyl(methyl)sulfamoyl]amino]-6-fluoro-phenoxy]-3-[6-[4-[3-(2,4-dioxohexahydropyrimidin-1-yl)-5-fluoro-1-methyl-indazol-6-yl]cyclohexyl]-6-azaspiro[3.4]octan-2-yl]-4-oxo-quinazoline (Compound 206, 13 mg, 14.59 mol, 3% yield) as an off-white solid. UPLC-MS (ES+): m/z 869.4 [M+H]⁺. ¹H NMR (400 MHz, DMSO-d₆): δ 10.55 (s, 1H), 8.36 (s, 1H), 7.75 (d, J=8.80 Hz, 1H), 7.59 (dd, J=2.80, 8.80 Hz, 1H), 7.45 (d, J=6.00 Hz, 1H), 7.38-7.32 (m, 3H), 7.24-7.22 (m, 1H), 7.21-7.08 (m, 1H), 4.89-4.85 (m, 1H), 3.99 (s, 3H), 3.95 (t, J=40.00 Hz, 2H), 3.25-3.18 (m, 3H), 2.99-2.94 (m, 4H), 2.75 (t, J=6.40 Hz, 3H), 2.54-2.51 (m, 6H), 2.02-1.95 (m, 7H), 1.74-1.55 (m, 4H), 1.02 (t, J=7.20 Hz, 3H).

### Synthesis D116: Synthesis of (3R)-8-[4-[3-(2,4-dioxohexahydropyrimidin-1-yl)-5-fluoro-1-methyl-indazol-6-yl]cyclohexyl]-3-[6-[3-[[ethyl(methyl)sulfamoyl]amino]-2,6-difluoro-benzoyl]-4-oxo-quinazolin-3-yl]-1-oxa-8-azaspiro[4.5]decane (Compound 207 and Compound 208)

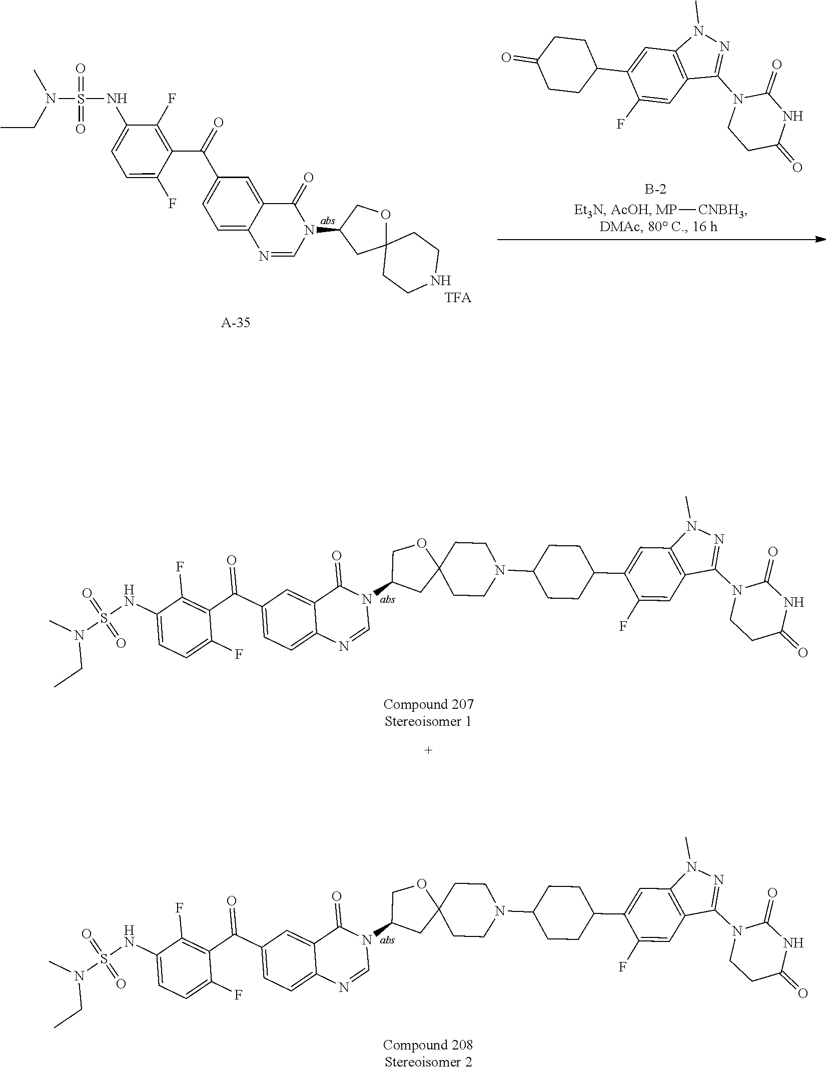

Into a 20 mL pressure vial containing a well-stirred solution of (3R)-3-[6-[3-[[ethyl(methyl)sulfamoyl]amino]-2,6-difluoro-benzoyl]-4-oxo-quinazolin-3-yl]-1-oxa-8-azaspiro[4.5]decane (A-35) (140 mg, 207.22 mol, TFA salt) in DMAc (4 mL) was added triethylamine (828.86 mol, 115.53 μL) at room temperature under nitrogen atmosphere and stirred for 20 min. Reaction mixture was cooled to 0° C., 1-[5-fluoro-1-methyl-6-(4-oxocyclohexyl)indazol-3-yl]hexahydropyrimidine-2,4-dione (B-2) (96.54 mg, 269.38 mol) and AcOH (414.43 mol, 23.70 μL) were added subsequently and stirred at room temperature for 3 hours. Finally, MP-CNBH₃ (140 mg, 207.22 mol) was added, and the reaction mixture was stirred at 80° C. for 16 hours. After completion of the reaction, the reaction mixture was filtered and the filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fractions were lyophilized to afford 90 mg of mixture of cis and trans isomers. Further cis and trans isomers were separated by preparative HPLC [Column: X-Bridge C18 (250 mm×19 mm×5 m), Mobile phase A: 10 mM NH₄HCO₃ in milli-Q water; Mobile phase B: acetonitrile]. The first eluting fraction (Peak-1) was lyophilized to afford Compound 207 (assigned as stereoisomer 1).

Peak-1: (3R)-8-[4-[3-(2,4-dioxohexahydropyrimidin-1-yl)-5-fluoro-1-methyl-indazol-6-yl]cyclohexyl]-3-[6-[3-[[ethyl(methyl)sulfamoyl]amino]-2,6-difluoro-benzoyl]-4-oxo-quinazolin-3-yl]-1-oxa-8-azaspiro[4.5]decane (Compound 207, 12 mg, 13.06 mol, 6% yield) as an off-white solid. UPLC-MS (ES+): m/z 904.3 [M+H]⁺. ¹H NMR (400 MHz, DMSO-d₆): δ 10.55 (s, 1H), 9.78 (bs, 1H), 8.53 (s, 1H), 8.40 (d, J=1.20 Hz, 1H), 8.30 (dd, J=2.00, 8.40 Hz, 1H), 7.88 (d, J=8.40 Hz, 1H), 7.72-7.66 (m, 1H), 7.54 (d, J=6.00 Hz, 1H), 7.37-7.31 (m, 2H), 5.30-5.27 (m, 1H), 4.17-4.09 (m, 2H), 3.99 (s, 3H), 3.90 (t, J=6.80 Hz, 2H), 3.11 (q, J=6.80 Hz, 2H), 2.83-2.68 (m, 8H), 2.50-2.38 (m, 4H), 2.06-2.02 (m, 1H), 1.88-1.93 (m, 4H), 1.53-1.82 (m, 6H), 1.45-1.53 (m, 2H), 1.02 (t, J=7.20 Hz, 3H).

Similarly, the second eluted fraction (Peak-2) was lyophilized to afford Compound 208 (assigned as stereoisomer 2).

Peak-2: (3R)-8-[4-[3-(2,4-dioxohexahydropyrimidin-1-yl)-5-fluoro-1-methyl-indazol-6-yl]cyclohexyl]-3-[6-[3-[[ethyl(methyl)sulfamoyl]amino]-2,6-difluoro-benzoyl]-4-oxo-quinazolin-3-yl]-1-oxa-8-azaspiro[4.5]decane (Compound 208, 15 mg, 16.48 mol, 8% yield) as a white solid. UPLC-MS (ES−): 902.4 [M−H]⁻. ¹H NMR (400 MHz, DMSO-d₆): δ 10.55 (s, 1H), 9.79 (bs, 1H), 8.54 (s, 1H), 8.39 (d, J=1.60 Hz, 1H), 8.30 (dd, J=2.40, 8.60 Hz, 1H), 7.88 (d, J=8.80 Hz, 1H), 7.72-7.66 (m, 1H), 7.45 (d, J=6.00 Hz, 1H), 7.37-7.33 (m, 2H), 5.32-5.23 (m, 1H), 4.15-4.09 (m, 2H), 4.01 (s, 3H), 3.91 (t, J=6.80 Hz, 2H), 3.14-3.09 (m, 2H), 3.03-2.92 (m, 1H), 2.77-2.67 (m, 5H), 2.40-2.39 (m, 2H), 2.49-2.42 (m, 2H), 2.31-2.28 (m, 1H), 2.12-2.03 (m, 3H), 1.97-1.92 (m, 2H), 1.74-1.62 (m, 4H), 1.60-1.49 (m, 4H), 1.02 (t, J=7.20 Hz, 3H)

### Synthesis D117: Synthesis of Synthesis of 6-[2-cyano-3-[[ethyl(methyl)sulfamoyl]amino]-6-fluoro-phenoxy]-3-[7-[3-[3-(2,4-dioxohexahydropyrimidin-1-yl)-5-fluoro-1-methyl-indazol-6-yl]cyclobutyl]-7-azaspiro[3.5]nonan-2-yl]-4-oxo-quinazoline (Compound 209)

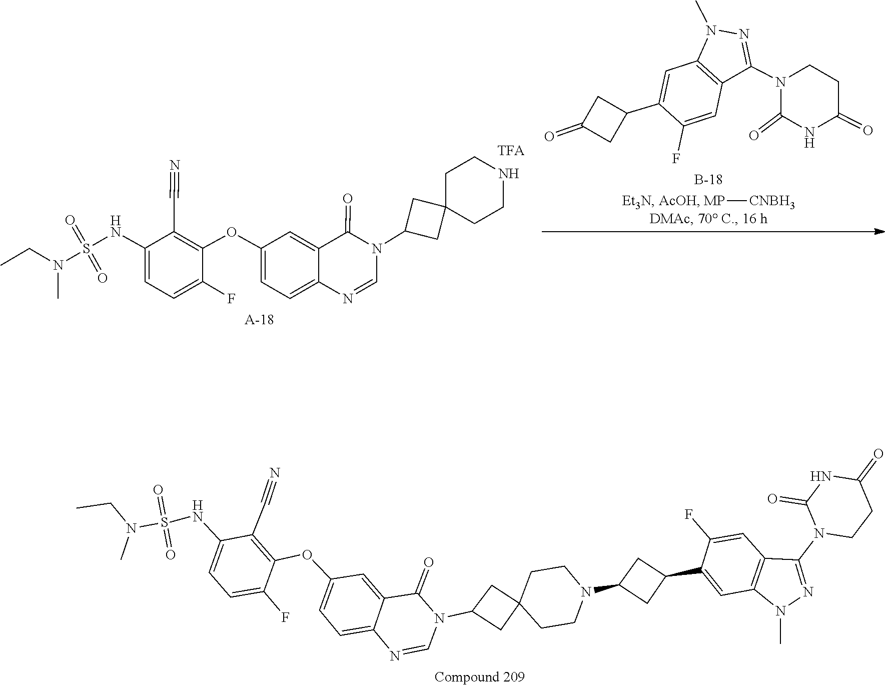

Into a 15 mL pressure vial containing a well-stirred solution of 3-(7-azaspiro[3.5]nonan-2-yl)-6-[2-cyano-3-[[ethyl(methyl)sulfamoyl]amino]-6-fluoro-phenoxy]-4-oxo-quinazoline (A-18) (140 mg, 186.06 mol, TFA salt) in DMAc (2 mL) was added triethylamine (930.29 mol, 129.66 L) and stirred for 1 hour. After 1 h, 1-[5-fluoro-1-methyl-6-(3-oxocyclobutyl)indazol-3-yl]hexahydropyrimidine-2,4-dione (B-18) (85.23 mg, 186.06 mol, TFA salt) and AcOH (558.18 mol, 31.92 μL) were added and stirred for 4 hours. Subsequently, MP-CNBH₃ (150 mg) was added and resulting mixture was stirred at 70° C. for 16 hours. After completion of the reaction, reaction mixture was filtered through sintered funnel and washed with dichloromethane. Filtrate was concentrated under reduced pressure to get crude residue which was purified by the reverse phase [Column: Redisep R_{f} Gold® reversed-phase C18-teledyne ISCO, 120 g), Mobile phase A: 10 mM NH₄HCO₃ in milli-Q-water; Mobile phase B: acetonitrile]. Fractions were collected and lyophilized to afford 6-[2-cyano-3-[[ethyl(methyl)sulfamoyl]amino]-6-fluoro-phenoxy]-3-[7-[3-[3-(2,4-dioxohexahydropyrimidin-1-yl)-5-fluoro-1-methyl-indazol-6-yl]cyclobutyl]-7-azaspiro[3.5]nonan-2-yl]-4-oxo-quinazoline (Compound 209, 2.7 mg, 3.10 mol, 2% yield) as an off-white solid. UPLC-MS (ES−): m/z 853.3 [M−H]⁻. ¹H NMR (400 MHz, DMSO-d₆): δ 10.56 (s, 1H), 9.40 (bs, 1H), 8.39 (s, 1H), 7.76 (d, J=8.80 Hz, 1H), 7.62 (dd, J=2.80, 8.80 Hz, 1H), 7.52 (d, J=6.00 Hz, 1H), 7.39-7.29 (m, 4H), 5.00-4.91 (m, 1H), 4.03 (s, 3H), 3.92 (t, J=6.80 Hz, 2H), 3.50-3.40 (m, 2H), 3.07-2.97 (m, 3H), 2.76 (t, J=6.80 Hz, 2H), 2.67-2.58 (m, 5H), 2.54-2.53 (m, 2H), 2.39-2.37 (m, 2H), 2.31-2.20 (m, 3H), 1.86-1.60 (m, 4H), 1.03 (t, J=7.20 Hz, 3H).

### Synthesis D118: Synthesis of 6-[2,6-difluoro-3-[[2-hydroxyethyl(methyl)sulfamoyl]amino]phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 210 and Compound 211)

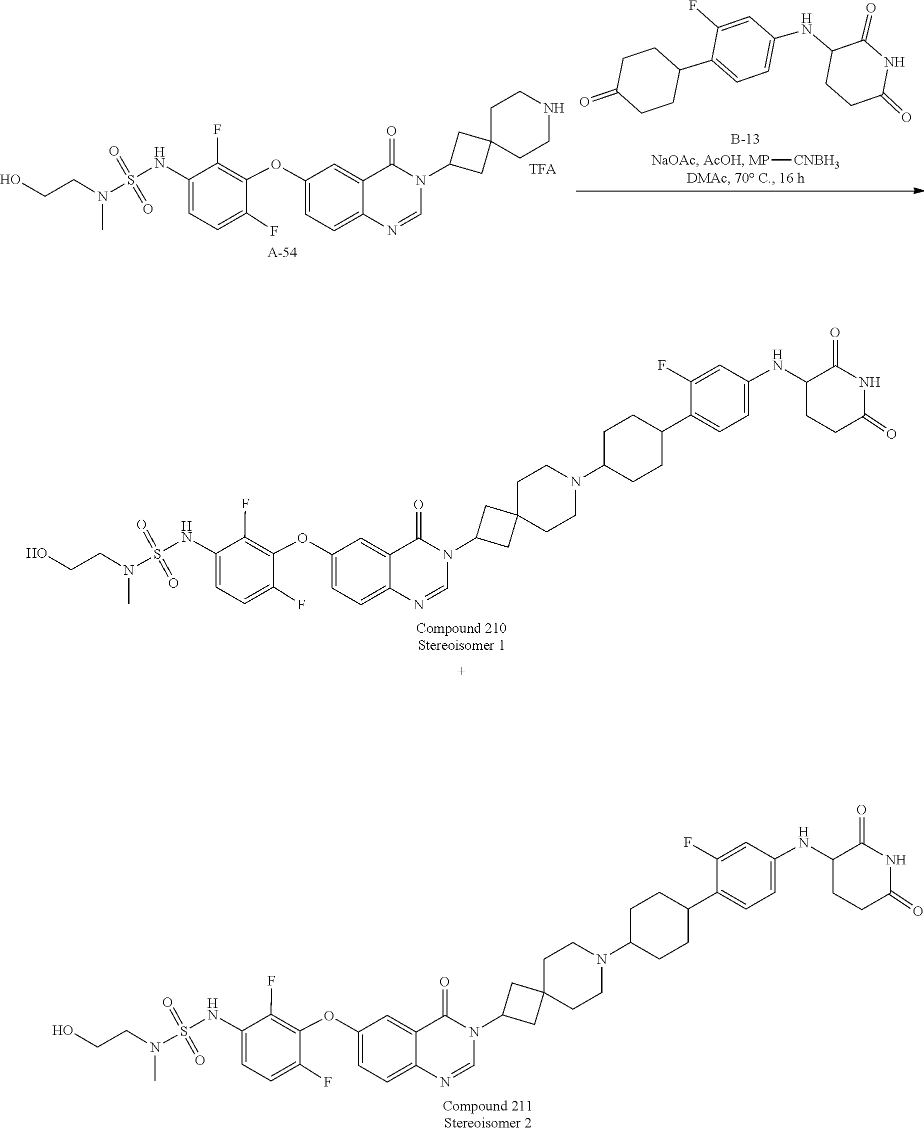

Into a 20 mL pressure vial containing a well-stirred solution of 3-(7-azaspiro[3.5]nonan-2-yl)-6-[2,6-difluoro-3-[[2-hydroxyethyl(methyl)sulfamoyl]amino]phenoxy]-4-oxo-quinazoline (A-54) (150 mg, 205.69 mol, TFA salt) in DMAc (2.01 mL) was added NaOAc (16.87 mg, 205.69 mol) at room temperature under nitrogen atmosphere and stirred for 30 min. Reaction mixture was cooled to 0° C. and was added 3-[3-fluoro-4-(4-oxocyclohexyl)anilino]piperidine-2,6-dione (B-13) (65.48 mg, 205.69 mol) followed by AcOH (617.08 mol, 35.29 μL) and resulting mixture was stirred for 2 hours at room temperature. Finally, MP-CNBH₃ (150 mg, 205.69 mol) was added, and the reaction mixture was stirred at 70° C. for 16 hours. After completion of the reaction, reaction mixture was filtered and filtrate was concentrated under reduced pressure to get a crude residue (200 mg) as a brown oil, which was purified by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction (Peak-1) was lyophilized to afford Compound 210 (assigned as stereoisomer 1).

Peak-1: 6-[2,6-difluoro-3-[[2-hydroxyethyl(methyl)sulfamoyl]amino]phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 210, 36.25 mg, 41.49 mol, 18% yield) as an off-white solid. UPLC-MS (ES−): m/z 850.3 [M−H]⁻. ¹H NMR (400 MHz, DMSO-d₆): δ 10.79 (s, 1H), 8.38 (s, 1H), 7.77 (d, J=8.80 Hz, 1H), 7.65 (d, J=2.80 Hz, 1H), 7.45-7.39 (m, 1H), 7.35-7.30 (m, 2H), 7.05 (d, J=2.80 Hz, 1H), 6.45-6.44 (m, 2H), 5.99 (d, J=8.00 Hz, 1H), 4.90 (t, J=8.40 Hz, 1H), 4.32-4.27 (m, 1H), 3.47 (t, J=6.00 Hz, 2H), 3.10 (t, J=6.00 Hz, 2H), 2.78 (s, 3H), 2.77-2.70 (m, 1H), 2.58 (t, J=9.60 Hz, 3H), 2.34-2.31 (m, 2H), 2.29-2.22 (m, 2H), 2.13-2.05 (m, 1H), 1.91-1.63 (m, 10H), 1.47-1.41 (m, 5H).

Similarly, the second eluted fraction (Peak-2) was lyophilized to afford Compound 211 (assigned as stereoisomer 2).

Peak-2: 6-[2,6-difluoro-3-[[2-hydroxyethyl(methyl)sulfamoyl]amino]phenoxy]-3-[7-[4-[4-[(2,6-dioxo-3-piperidyl)amino]-2-fluoro-phenyl]cyclohexyl]-7-azaspiro[3.5]nonan-2-yl]-4-oxo-quinazoline (Compound 211, 38.49 mg, 44.05 mol, 19% yield) as an off-white solid. UPLC-MS (ES+): m/z 852.2 [M+H]⁺. ¹H NMR (400 MHz, DMSO-d₆): δ 10.79 (s, 1H), 9.64 (bs, 1H), 8.38 (s, 1H), 7.77 (d, J=8.80 Hz, 1H), 7.65 (d, J=2.80 Hz, 1H), 7.50-7.30 (m, 3H), 7.05 (d, J=2.80 Hz, 1H), 6.45-6.44 (m, 2H), 5.99 (d, J=8.00 Hz, 1H), 4.90 (t, J=8.40 Hz, 1H), 4.32-4.27 (m, 1H), 3.47 (t, J=6.00 Hz, 2H), 3.10 (t, J=6.00 Hz, 2H), 2.78 (s, 3H), 2.78-2.68 (m, 2H), 2.34-2.31 (m, 2H), 2.34-2.30 (m, 3H), 1.91-1.66 (m, 11H), 1.50-1.42 (m, 4H).

### Synthesis D119: Synthesis of 6-[2-cyano-6-fluoro-3-[[2-hydroxyethyl(methyl)sulfamoyl]amino]phenoxy]-3-[(3R)-8-[(4S)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 212 and Compound 213)

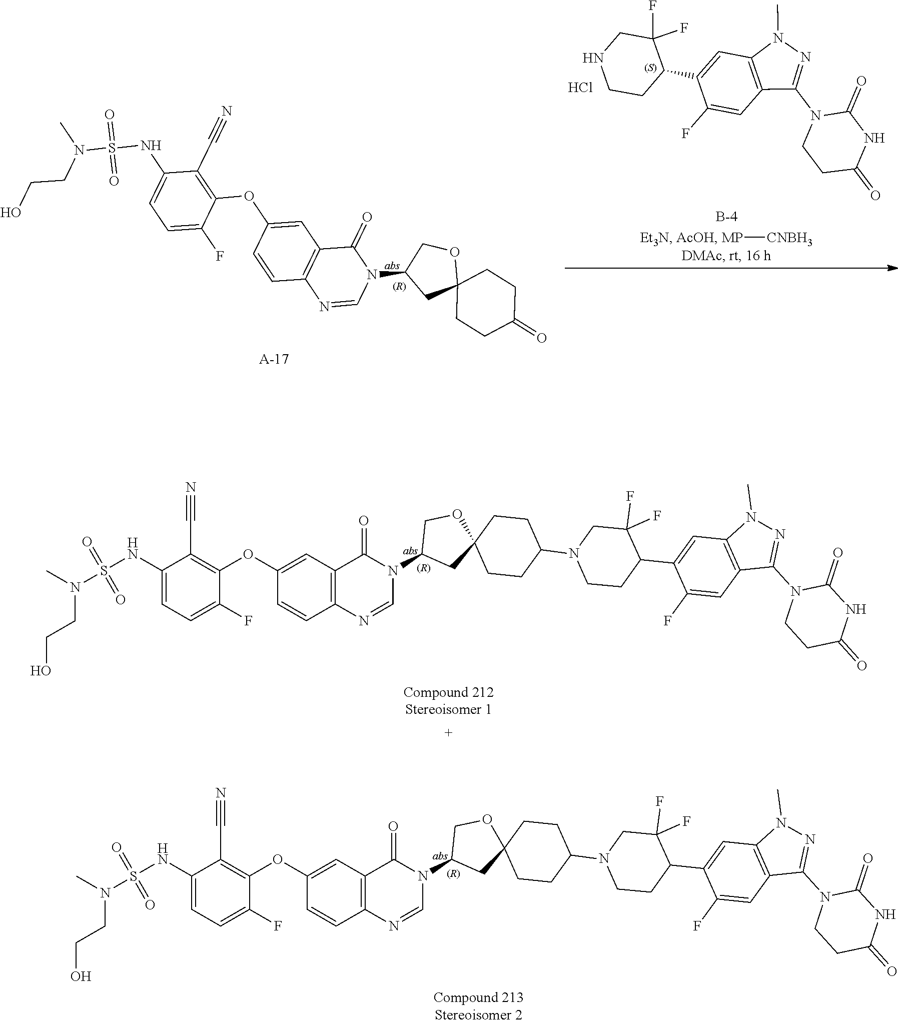

Into a 20 mL pressure vial containing a well-stirred solution 1-[6-[(4S)-3,3-difluoro-4-piperidyl]-5-fluoro-1-methyl-indazol-3-yl]hexahydropyrimidine-2,4-dione (B-4) (300 mg, 718.03 mol, HCl salt) in DMAc (4 mL) was added triethylamine (2.87 mmol, 400.31 μL) at room temperature under nitrogen atmosphere and stirred for 20 min. Reaction mixture was cooled to 0° C., subsequently, 6-[2-cyano-6-fluoro-3-[[2-hydroxyethyl(methyl)sulfamoyl]amino]phenoxy]-4-oxo-3-[(3R)-8-oxo-1-oxaspiro[4.5]decan-3-yl]quinazoline (A-17) (462.53 mg, 743.53 mol) and AcOH (91.64 mol, 5.23 μL) were added and stirred for 3 hours at room temperature. Finally, MP-CNBH₃ (300 mg, 718.03 mol) was added, and the reaction mixture was stirred at room temperature for 16 hours. After completion of the reaction, reaction mixture was filtered and the filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fractions were lyophilized to afford 120 mg of mixture of R and S isomers. Further, R and S isomers were separated by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction (Peak-1) was lyophilized to afford Compound 212 (assigned as stereoisomer 1).

Peak-1: 6-[2-cyano-6-fluoro-3-[[2-hydroxyethyl(methyl)sulfamoyl]amino]phenoxy]-3-[(3R)-8-[(4S)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 212, 56 mg, 56.01 mol, 8% yield) as an off-white solid. UPLC-MS (ES+): m/z 951.3 [M+H]⁺. ¹H NMR (400 MHz, DMSO-d₆): δ 10.56 (s, 1H), 10.13 (bs, 1H), 8.37 (s, 1H), 7.82 (t, J=8.80 Hz, 1H), 7.77 (d, J=18.80 Hz, 1H), 7.73-7.68 (m, 2H), 7.54 (d, J=4.00 Hz, 1H), 7.43-7.40 (m, 2H), 5.32-5.21 (m, 1H), 4.85 (s, 1H), 4.15-4.04 (m, 2H), 4.02 (s, 3H), 3.92 (t, J=6.80 Hz, 2H), 3.52-3.47 (m, 2H), 3.19 (t, J=6.00 Hz, 2H), 3.28-3.11 (m, 3H), 3.08-3.02 (m, 1H), 2.87 (s, 3H), 2.76 (t, J=6.80 Hz, 2H), 2.45-2.42 (m, 1H), 2.34-2.25 (m, 1H), 2.05-2.00 (m, 1H), 1.91-1.75 (m, 5H), 1.64-1.58 (m, 2H), 1.44-1.29 (m, 3H).

Similarly, the second eluted fraction (Peak-2) was lyophilized to afford Compound 213 (assigned as stereoisomer 2).

Peak-2: 6-[2-cyano-6-fluoro-3-[[2-hydroxyethyl(methyl)sulfamoyl]amino]phenoxy]-3-[(3R)-8-[(4S)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 213, 16 mg, 16.51 mol, 2% yield) as a white solid. UPLC-MS (ES+): m/z 951.4 [M+H]⁺. ¹H NMR (400 MHz, DMSO-d₆): δ 10.57 (s, 1H), 10.13 (s, 1H), 8.36 (s, 1H), 7.88-7.68 (m, 4H), 7.53-7.48 (m, 1H), 7.44-7.39 (m, 2H), 5.31-5.22 (m, 1H), 4.84 (s, 1H), 4.15-4.02 (m, 5H), 3.92 (t, J=6.80 Hz, 2H), 3.51-3.45 (m, 3H), 3.18 (t, J=5.60 Hz, 3H), 3.04-2.85 (m, 1H), 2.86 (s, 3H), 2.76 (t, J=6.40 Hz, 2H), 2.71-2.69 (m, 2H), 2.68-2.66 (m, 2H), 2.28-2.25 (m, 1H), 2.02-1.87 (m, 4H), 1.67-1.54 (m, 5H), 1.39-1.33 (m, 1H).

### Synthesis D120: Synthesis of 6-[2-cyano-6-fluoro-3-[[2-hydroxyethyl(methyl)sulfamoyl]amino]phenoxy]-3-[(3R)-8-[(4R)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 214 and Compound 215)

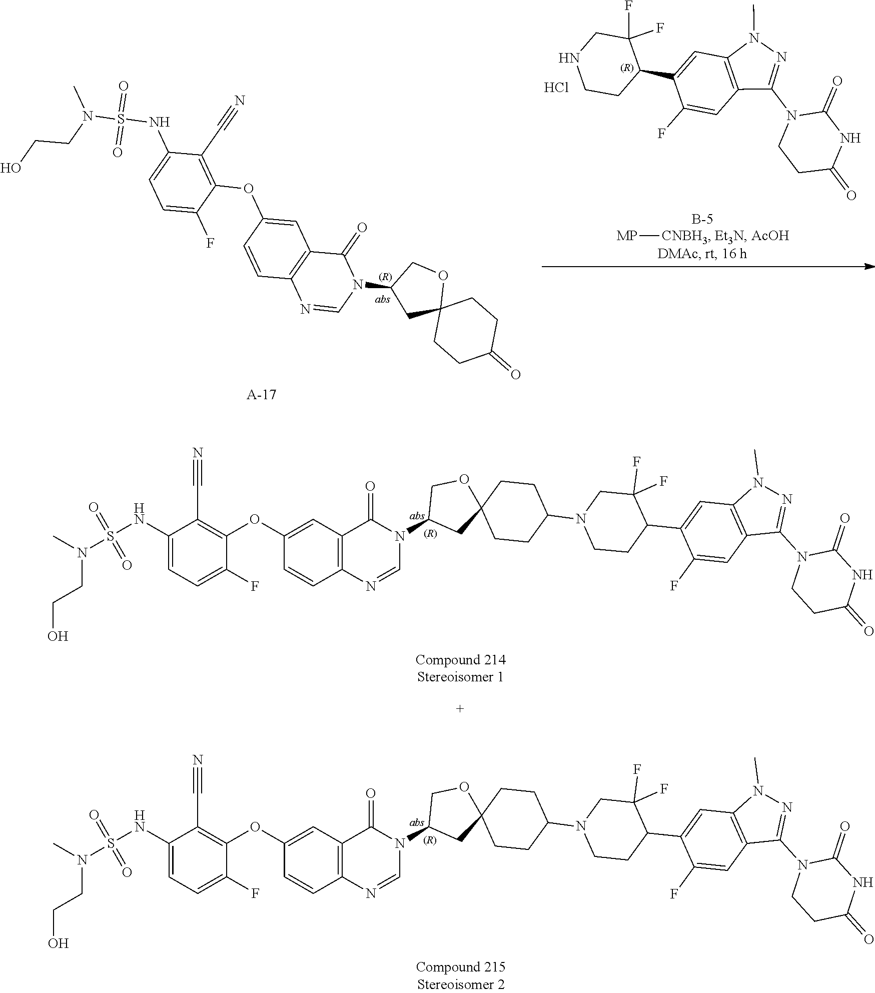

Into a 20 mL pressure vial containing a well-stirred solution 1-[6-[(4R)-3,3-difluoro-4-piperidyl]-5-fluoro-1-methyl-indazol-3-yl]hexahydropyrimidine-2,4-dione (B-5) (300 mg, 718.03 mol, HCl salt) of in DMAc (4 mL) was added triethylamine (2.15 mmol, 300.24 μL) at room temperature under nitrogen atmosphere and stirred for 20 min. Reaction mixture was cooled to 0° C., Subsequently, 6-[2-cyano-6-fluoro-3-[[2-hydroxyethyl(methyl)sulfamoyl]amino]phenoxy]-4-oxo-3-[(3R)-8-oxo-1-oxaspiro[4.5]decan-3-yl]quinazoline (A-17) (618.35 mg, 718.03 mol) and AcOH (91.64 mol, 5.23 μL) were added and stirred for 3 hours at room temperature. Finally, MP-CNBH₃ (300 mg, 718.03 mol) was added, and the reaction mixture was stirred at room temperature for 16 hours. After completion of the reaction, reaction mixture was filtered and the filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fractions were lyophilized to afford 120 mg of mixture of R and Sisomers. Further R and S isomers were separated by preparative HPLC [Column: X-Bridge C18 (250 mm×19 mm×5 m), Mobile phase A: 10 mM NH₄HCO₃ in milli-Q water; Mobile phase B: acetonitrile]. The first eluting fraction (Peak-1) was lyophilized to afford Compound 214 (assigned as stereoisomer 1).

Peak-1: 6-[2-cyano-6-fluoro-3-[[2-hydroxyethyl(methyl)sulfamoyl]amino]phenoxy]-3-[(3R)-8-[(4R)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 214, 15 mg, 15.58 mol, 2% yield) as an off-white solid. UPLC-MS (ES+): m/z 951.2 [M+H]⁺. ¹H NMR (400 MHz, DMSO-d₆): δ 10.56 (s, 1H), 10.13 (bs, 1H), 8.36 (s, 1H), 7.80-7.73 (m, 2H), 7.67 (dd, J=2.80, 8.80 Hz, 1H), 7.46-7.39 (m, 3H), 5.31-5.25 (m, 1H), 5.02 (s, 1H), 4.15-4.06 (m, 2H), 4.02 (s, 3H), 3.91 (t, J=6.80 Hz, 2H), 3.51-3.43 (m, 3H), 3.33-3.19 (m, 3H), 3.65-3.02 (m, 1H), 2.84-2.76 (m, 2H), 2.76 (t, J=6.80 Hz, 2H), 2.53-2.49 (m, 2H), 2.46-2.40 (m, 2H), 2.12-1.99 (m, 1H), 1.92-1.76 (m, 4H), 1.64-1.58 (m, 1H), 1.44-1.36 (m, 3H).

Similarly, the second eluted fraction (Peak-2) was lyophilized to afford Compound 215 (assigned as stereoisomer 2).

Peak-2: 6-[2-cyano-6-fluoro-3-[[2-hydroxyethyl(methyl)sulfamoyl]amino]phenoxy]-3-[(3R)-8-[(4R)-4-[3-(2,4-dioxohexahydropyrimidin-1-yl)-5-fluoro-1-methyl-indazol-6-yl]-3,3-difluoro-1-piperidyl]-1-oxaspiro[4.5]decan-3-yl]-4-oxo-quinazoline (Compound 215, 9 mg, 9.38 mol, 1% yield) as a white solid. UPLC-MS (ES+): m/z 951.2 [M+H]⁺. ¹H NMR (400 MHz, DMSO-d₆): δ 10.57 (s, 1H), 10.13 (bs, 1H), 8.36 (s, 1H), 7.91-7.78 (m, 3H), 7.69 (dd, J=2.80, 8.80 Hz, 1H), 7.53-7.48 (m, 1H), 7.43-7.38 (m, 2H), 5.31-5.26 (m, 1H), 4.83 (s, 1H), 4.14-4.06 (m, 2H), 4.03 (s, 3H), 3.92 (t, J=6.80 Hz, 2H), 3.51-3.33 (m, 3H), 3.23-3.17 (m, 3H), 3.19-3.02 (m, 1H), 2.92-2.81 (m, 3H), 2.68 (t, J=2.00 Hz, 3H), 2.34-2.25 (m, 2H), 2.02-1.83 (m, 4H), 1.69-1.52 (m, 5H), 1.42-1.31 (m, 1H).

### Synthesis D121: Synthesis of (3-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-8-[3-(2,4-dioxohexahydropyrimidin-1-yl)-5-fluoro-1-methyl-indazol-6-yl]-1-oxa-8-azaspiro[4.5]decane (Compound 216)

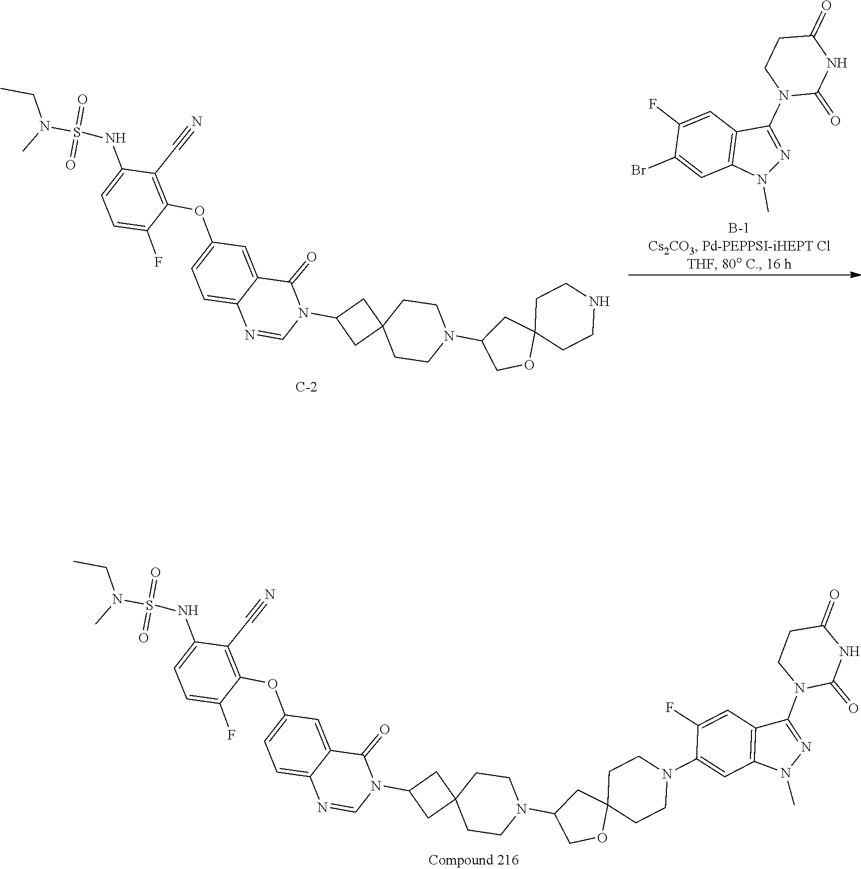

Into a 40 mL pressure vial containing a well-stirred solution of 3-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-1-oxa-8-azaspiro[4.5]decane (C-2, 200.00 mg, 294.20 mol) in anhydrous THF (3 mL) was added Cs₂CO₃ (287.57 mg, 882.61 mol) at room temperature under nitrogen atmosphere and the resulting mixture was degassed by bubbling nitrogen gas into the reaction mixture for 10 minutes. Subsequently, 1-(6-bromo-5-fluoro-1-methyl-indazol-3-yl)hexahydropyrimidine-2,4-dione (B-1) (111.51 mg, 294.20 mol) was added to the reaction mixture and reaction mixture was again degassed by bubbling nitrogen gas into the reaction mixture for 10 minutes. Finally, Pd-Peppsi-iheptCl (14.30 mg, 14.71 mol) was added at room temperature and the reaction mixture was stirred at 80° C. for 16 hours. After completion of the reaction, the reaction mixture was filtered and the filtrate was concentrated under reduced pressure to get a crude residue which was purified by preparative HPLC [Column: X-BRIDGE C18 (150 mm*19 mm*5 μm), Mobile phase A: 10 mm NH₄HCO₃ in milli-Q water; Mobile phase B: acetonitrile]. The eluting fraction was concentrated under reduced pressure at 40° C. and lyophilized to afford (3-[2-[6-[2-cyano-3-[[ethyl(methyl)sulfamoyl]amino]-6-fluoro-phenoxy]-4-oxo-quinazolin-3-yl]-7-azaspiro[3.5]nonan-7-yl]-8-[3-(2,4-dioxohexahydropyrimidin-1-yl)-5-fluoro-1-methyl-indazol-6-yl]-1-oxa-8-azaspiro[4.5]decane (Compound 216, 10 mg, 10.58 mol, 4% yield) as a white solid. UPLC-MS (ES+): m/z 940.3 [M+H]⁺. ¹H NMR (400 MHz, DMSO-d₆): δ 10.53 (s, 1H), 9.86 (s, 1H), 8.39 (s, 1H), 7.77 (d, J=8.80 Hz, 1H), 7.75-7.23 (m, 5H), 7.13 (d, J=7.20 Hz, 1H), 5.20-4.85 (m, 1H), 4.08-3.83 (m, 6H), 3.83-3.59 (m, 1H), 3.17-2.99 (m, 6H), 2.74 (t, J=6.80 Hz, 2H), 2.68-2.62 (m, 2H), 2.55-2.53 (m, 4H), 2.45-2.37 (m, 2H), 2.32-2.09 (m, 4H), 1.96-1.51 (m, 10H), 1.04 (t, J=7.20 Hz, 3H).

### Synthesis D122: Synthesis of 6-[2-cyano-3-[[ethyl(methyl)sulfamoyl]amino]-6-fluoro-phenoxy]-3-[2-[(4R)-4-[3-(2,4-dioxohexahydropyrimidin-1-yl)-5-fluoro-1-methyl-indazol-6-yl]-3,3-difluoro-1-piperidyl]spiro[3.3]heptan-6-yl]-4-oxo-quinazoline (Compound 217)

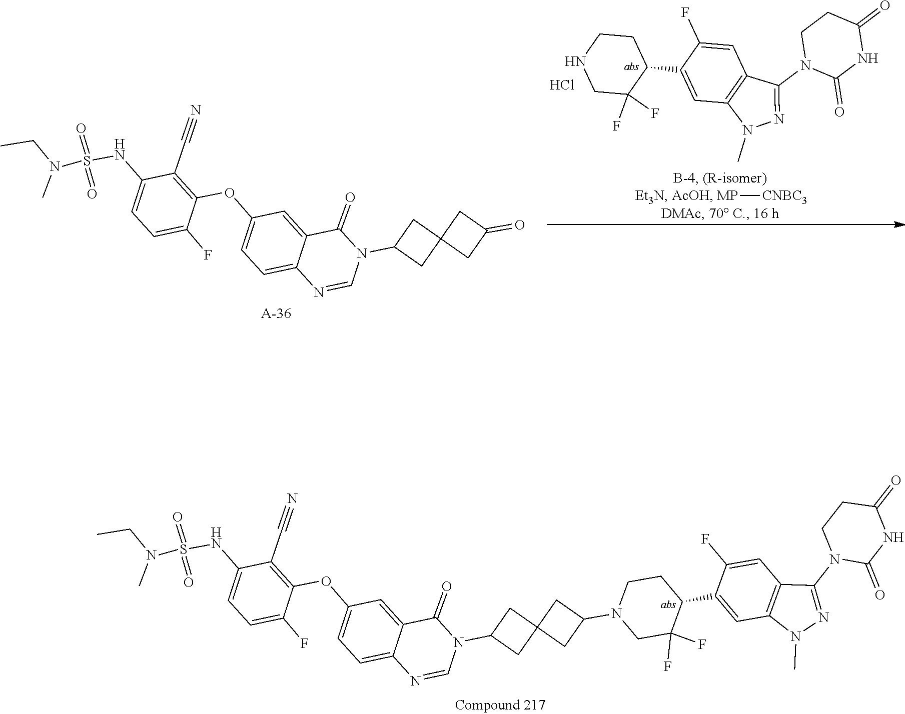

Into a 8 mL pressure vial containing a well-stirred solution of 1-[6-[(4R)-3,3-difluoro-4-piperidyl]-5-fluoro-1-methyl-indazol-3-yl]hexahydropyrimidine-2,4-dione (B-4) (35.77 mg, 85.62 mol, HCl salt) in DMAc (3 mL) was added triethylamine (256.87 mol, 35.80 μL) at room temperature under nitrogen atmosphere and mixture was stirred for 20 minutes. Reaction mixture was cooled to 0° C. and was added 6-[2-cyano-3-[[ethyl(methyl)sulfamoyl]amino]-6-fluoro-phenoxy]-4-oxo-3-(2-oxospiro[3.3]heptan-6-yl)quinazoline (A-36) (60.81 mg, 85.62 mol) followed by AcOH (428.12 mol, 24.48 μL). The resulting mixture was stirred for 3 hours at room temperature. Finally, MP-CNBH₃ (45 mg, 85.62 mol) was added, and the reaction mixture was stirred at 70° C. for 16 hours. After completion of the reaction, reaction mixture was filtered and the filtrate was concentrated under reduced pressure to get a crude residue which was purified by reverse phase column chromatography (Redisep R_{f} Gold®reversed-phase C18-teledyne ISCO, 120 g), [Mobile phase A: 10 mM NH₄HCO₃ in milli-Q water; Mobile phase B: acetonitrile]. The fractions were lyophilized to afford 30 mg of product, which is impure. Further separated by preparative HPLC [Column: X-Bridge C18 (150 mm*19 mm*5 m), Mobile phase A: 10 mM NH₄HCO₃ in milli-Q water; Mobile phase B: acetonitrile]. The eluting fraction was concentrated under reduced pressure at 40° C. and lyophilized to afford 6-[2-cyano-3-[[ethyl(methyl)sulfamoyl]amino]-6-fluoro-phenoxy]-3-[2-[(4R)-4-[3-(2,4-dioxohexahydropyrimidin-1-yl)-5-fluoro-1-methyl-indazol-6-yl]-3,3-difluoro-1-piperidyl]spiro[3.3]heptan-6-yl]-4-oxo-quinazoline (Compound 217, 17.3 mg, 19.40 mol, 23% yield) as a white solid. UPLC-MS (ES+): m/z 891.2 [M+H]⁺. ¹H NMR (400 MHz, DMSO-d₆): δ 10.57 (s, 1H), 10.18 (s, 1H), 8.37 (d, J=0.8 Hz, 1H), 7.79-7.72 (m, 3H), 7.71-7.63 (m, 1H), 7.52-7.41 (m, 2H), 7.34 (d, J=2.8 Hz, 1H), 4.79 (quint, J=8.0 Hz, 1H), 4.02 (s, 3H), 3.92 (t, J=6.8 Hz, 2H), 3.59-3.46 (m, 1H), 3.20-3.03 (m, 3H), 3.02-2.88 (m, 2H), 2.81-2.72 (m, 5H), 2.61-2.53 (m, 2H), 2.49-2.33 (m, 4H), 2.32-2.24 (m, 1H), 2.20-2.04 (m, 2H), 2.03-1.80 (m, 3H), 1.05 (t, J=7.2 Hz, 3H).

### Synthesis D123: Synthesis of 7-[2-cyano-3-[[ethyl(methyl)sulfamoyl]amino]-6-fluoro-phenoxy]-2-[[7-[4-[3-(2,4-dioxohexahydropyrimidin-1-yl)-5-fluoro-1-methyl-indazol-6-yl]cyclohexyl]-7-azaspiro[3.5]nonan-2-yl]oxy]quinoxaline (Compound 218 and Compound 219)

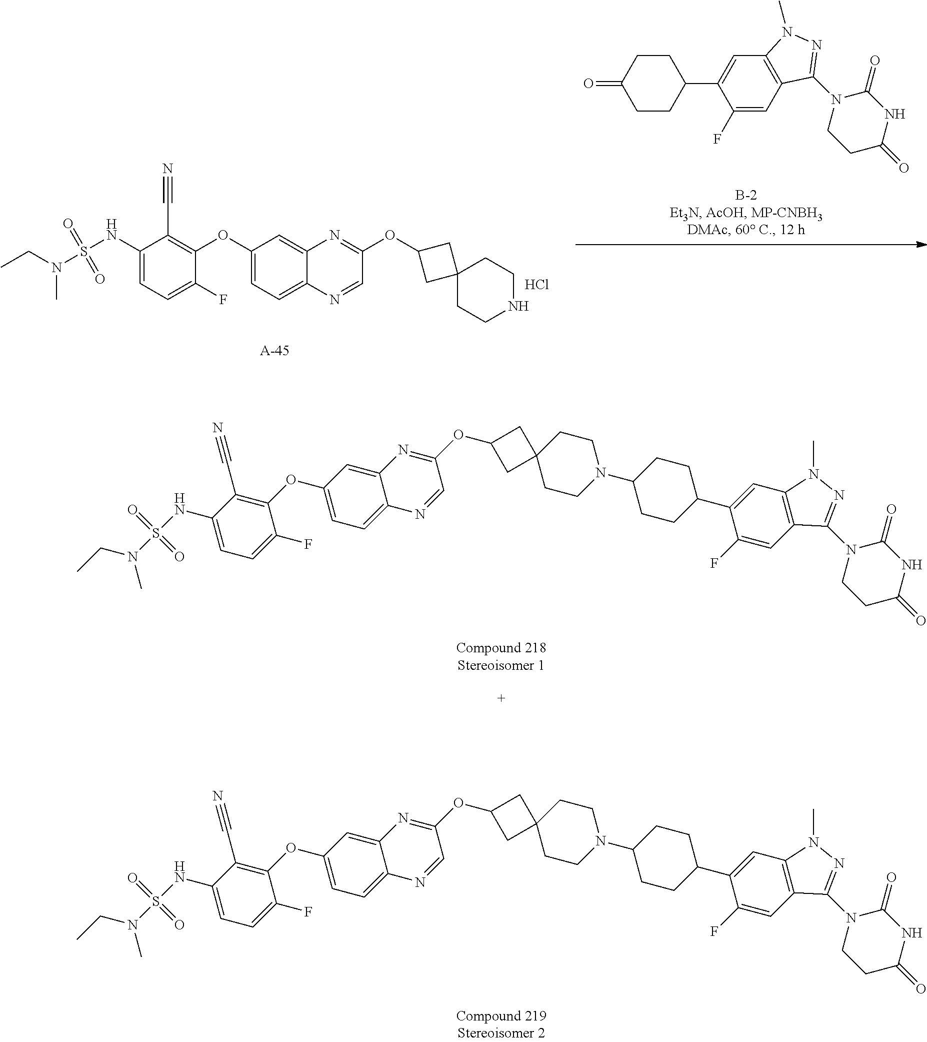

Into a 20 mL glass vial with pressure released cap containing a well-stirred solution of 2-(7-azaspiro[3.5]nonan-2-yloxy)-7-[2-cyano-3-[[ethyl(methyl)sulfamoyl]amino]-6-fluoro-phenoxy]quinoxaline (A-45) (650 mg, 1.08 mmol, HCl salt) in DMAc (10 mL) was added triethylamine (3.24 mmol, 452.14 μL) at room temperature and stirred for 5 min. Subsequently, AcOH (2.16 mmol, 123.68 μL) amd 1-[5-fluoro-1-methyl-6-(4-oxocyclohexyl)indazol-3-yl]hexahydropyrimidine-2,4-dione (B-2) (465.01 mg, 1.30 mmol) were added at room temperature and stirred for 2 hours. Finally, MP-CNBH₃ (650 mg, 1.08 mmol) was added at room temperature. Resulting reaction mixture was stirred at 60° C. for 12 hours. After completion of the reaction, the reaction mixture was filtered through cotton pad and filtrate was concentrated under reduced pressure to get a crude residue which was purified by prep HPLC by using a method Column: X Bridge C18 (150 mm×19 mm×5 μm) and [Mobile phase A: 10 mm NH₄HCO₃ (pH-10 adjusted with NH₄OH) in milli-Q water; Mobile phase B: acetonitrile]. First eluted fraction (Peak-1) was lyophilized to afford Compound 218 (assigned as stereoisomer 1).

Peak-1: 7-[2-cyano-3-[[ethyl(methyl)sulfamoyl]amino]-6-fluoro-phenoxy]-2-[[7-[4-[3-(2,4-dioxohexahydropyrimidin-1-yl)-5-fluoro-1-methyl-indazol-6-yl]cyclohexyl]-7-azaspiro[3.5]nonan-2-yl]oxy]quinoxaline (Compound 218, 150 mg, 165.62 mol, 15% yield) as an off-white solid. UPLC-MS (ES+): m/z 883.3 [M+H]⁺. ¹H NMR (400 MHz, DMSO-d₆): δ 10.56 (s, 1H), 8.91 (bs, 1H), 8.49 (s, 1H), 8.05 (d, J=9.20 Hz, 1H), 7.55-7.28 (m, 5H), 6.86 (bs, 1H), 5.33-5.30 (m, 1H), 4.03 (s, 3H), 3.91 (t, J=6.40 Hz, 2H), 3.33-2.89 (m, 8H), 2.76 (t, J=6.40 Hz, 2H), 2.67-2.51 (m, 3H), 2.20-1.60 (m, 16H), 1.04 (t, J=7.20 Hz, 3H).

Similarly, the second eluted fraction (Peak-2) was lyophilized to afford Compound 219 (assigned as stereoisomer 2).

Peak-2: 7-[2-cyano-3-[[ethyl(methyl)sulfamoyl]amino]-6-fluoro-phenoxy]-2-[[7-[4-[3-(2,4-dioxohexahydropyrimidin-1-yl)-5-fluoro-1-methyl-indazol-6-yl]cyclohexyl]-7-azaspiro[3.5]nonan-2-yl]oxy]quinoxaline (Compound 219, 160 mg, 173.54 mol, 16% yield) as a white solid. UPLC-MS (ES+): m/z 883.3 [M+H]⁺. ¹H NMR (400 MHz, DMSO-d₆): δ 10.57 (s, 1H), 8.49-8.48 (m, 2H), 8.05 (d, J=9.20 Hz, 1H), 7.53-7.38 (m, 4H), 7.30-7.28 (m, 1H), 6.87 (bs, 1H), 5.34-5.27 (m, 1H), 4.03 (s, 3H), 3.92 (t, J=6.80 Hz, 2H), 3.50-3.30 (m, 2H), 3.15-2.99 (m, 5H), 2.76 (t, J=6.80 Hz, 2H), 2.60-2.55 (m, 5H), 2.53-2.50 (m, 1H), 2.10-1.70 (m, 14H), 1.04 (t, J=7.20 Hz, 3H).

### Synthesis D124: Synthesis of 6-[2-cyano-3-[[ethyl(methyl)sulfamoyl]amino]-6-fluoro-phenoxy]-3-[7-[2-[3-(2,4-dioxohexahydropyrimidin-1-yl)-5-fluoro-1-methyl-indazol-6-yl]-2-azaspiro[3.5]nonan-7-yl]-7-azaspiro[3.5]nonan-2-yl]-4-oxo-quinazoline (Compound 220)

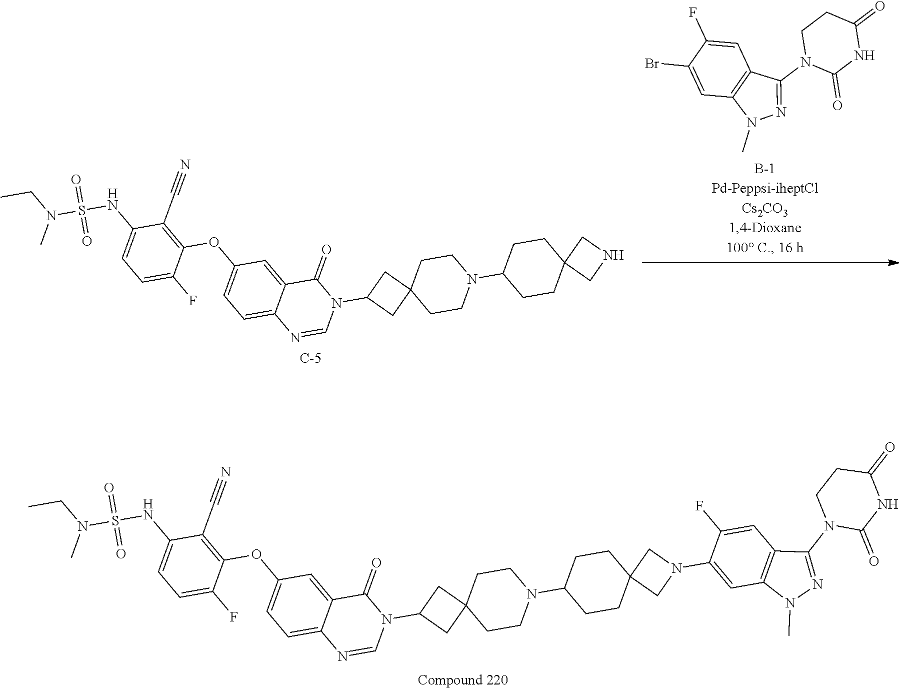

Into a 30 mL pressure vial containing a well-stirred solution of 3-[7-(2-azaspiro[3.5]nonan-7-yl)-7-azaspiro[3.5]nonan-2-yl]-6-[2-cyano-3-[[ethyl(methyl)sulfamoyl]amino]-6-fluoro-phenoxy]-4-oxo-quinazoline (C-5) 300 mg, 451.94 mol) in anhydrous 1,4-dioxane (10 mL) were added 1-(6-bromo-5-fluoro-1-methyl-indazol-3-yl)hexahydropyrimidine-2,4-dione (B-1) (231.26 mg, 677.91 mol) and Cs₂CO₃ (441.75 mg, 1.36 mmol) under nitrogen atmosphere, degassed for 5 min by bubbling nitrogen gas into the reaction mixture. Finally, Pd-Peppsi-iheptCl (43.93 mg, 45.19 mol) was added and further degassed for 2 min and stirred at 100° C. for 16 hours. After completion of the reaction, the reaction mixture was filtered, filtrate was concentrated under reduced pressure to get a crude residue, which was purified by prep HPLC [Column: X-Bridge C18 (150 mm*19 mm*5 m), Mobile phase A: 10 mm NH₄HCO₃ in milli-Q water; Mobile phase B: acetonitrile]. The eluting fraction was concentrated under reduced pressure at 40° C. and lyophilized to afford 6-[2-cyano-3-[[ethyl(methyl)sulfamoyl]amino]-6-fluoro-phenoxy]-3-[7-[2-[3-(2,4-dioxohexahydropyrimidin-1-yl)-5-fluoro-1-methyl-indazol-6-yl]-2-azaspiro[3.5]nonan-7-yl]-7-azaspiro[3.5]nonan-2-yl]-4-oxo-quinazoline (Compound 220, 18.78 mg, 20.13 mol, 4% yield) as an white solid. UPLC-MS (ES+): m/z 924.4 [M+H]⁺. ¹H NMR (400 MHz, DMSO-d₆): δ 10.50 (s, 1H), 8.81 (s, 1H), 8.38 (s, 1H), 7.75 (d, J=8.8 Hz, 1H), 7.62 (dd, J=2.8 Hz, 8.8 Hz, 1H), 7.49-7.10 (m, 4H), 6.49 (d, J=7.6 Hz, 1H), 4.95 (quint, J=8.8 Hz, 1H), 3.96-3.82 (m, 5H), 3.81-3.73 (m, 2H), 3.71-3.61 (m, 2H), 3.03-2.96 (m, 3H), 2.79-2.71 (m, 2H), 2.65-2.54 (m, 6H), 2.49-2.34 (m, 3H), 2.33-2.20 (m, 2H), 2.19-1.68 (m, 8H), 1.67-1.41 (m, 4H), 1.02 (t, J=7.2 Hz, 3H).

### Synthesis D125: Synthesis of 6-[2-cyano-3-[[ethyl(methyl)sulfamoyl]amino]-6-fluoro-phenoxy]-3-[4-[7-[3-(2,4-dioxohexahydropyrimidin-1-yl)-5-fluoro-1-methyl-indazol-6-yl]-2,7-diazaspiro[3.5]nonan-2-yl]cyclohexyl]-4-oxo-quinazoline (Compound 221 and Compound 222)

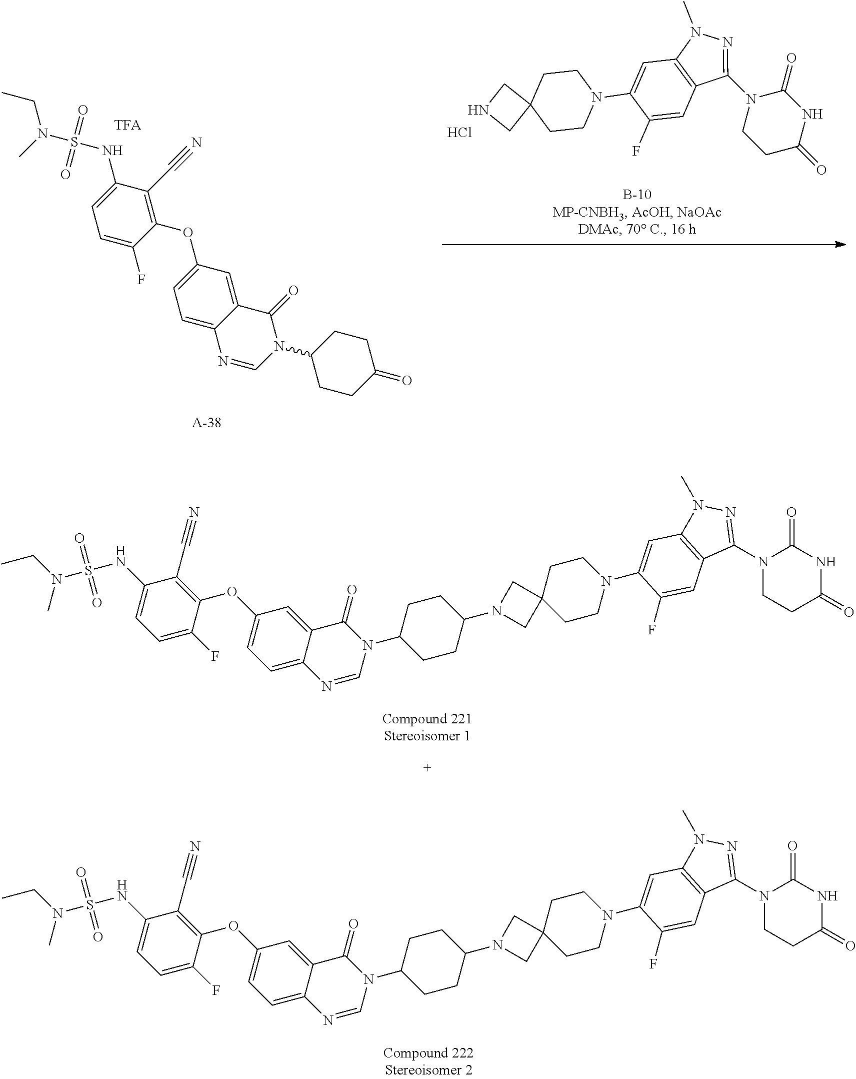

Into a 20 mL glass pressure vial containing a well-stirred solution of 6-[2-cyano-3-[[ethyl(methyl)sulfamoyl]amino]-6-fluoro-phenoxy]-4-oxo-3-(4-oxocyclohexyl)quinazoline 1 (A-38) (1.78 g, 2.84 mmol, TFA salt) in DMAc (10 mL) was added NaOAc (775.91 mg, 9.46 mmol) at room temperature under nitrogen atmosphere and the mixture was stirred for 1 hour. Subsequently, 1-[6-(2,7-diazaspiro[3.5]nonan-7-yl)-5-fluoro-1-methyl-indazol-3-yl]hexahydropyrimidine-2,4-dione 2 (B-10) (0.800 g, 1.89 mmol, HCl salt) and AcOH (5.68 mmol, 324.57 μL) were added and the resulting mixture was stirred at room temperature for 2 hours. Finally, MP-CNBH₃ (1.4 g, 1.89 mmol) was added, and the reaction mixture was stirred at 70° C. for 16 hours. After completion of the reaction, the reaction mixture was filtered and the filtrate was concentrated under reduced pressure to get a crude residue which was purified by preparative HPLC [Column: X-Bridge C18 (150 mm×21 mm×5 m), Mobile phase A: 10 mM NH₄HCO₃ in milli-Q water (pH was adjusted to 10 with ammonia); Mobile phase B: acetonitrile]. The first eluting fraction was concentrated under reduced pressure at 40° C. and lyophilized to afford Compound 221 (assigned as stereoisomer 1).

Peak-1: 6-[2-cyano-3-[[ethyl(methyl)sulfamoyl]amino]-6-fluoro-phenoxy]-3-[4-[7-[3-(2,4-dioxohexahydropyrimidin-1-yl)-5-fluoro-1-methyl-indazol-6-yl]-2,7-diazaspiro[3.5]nonan-2-yl]cyclohexyl]-4-oxo-quinazoline (Compound 221, 0.033 g, 37.31 mol, 2% yield) as a white solid. UPLC-MS (ES+): m/z 884.3 [M+H]⁺. ¹H NMR (400 MHz, DMSO-d₆): δ 10.53 (s, 1H), 9.83 (bs, 1H), 8.38 (s, 1H), 7.71 (d, J=12.00 Hz, 1H), 7.62 (dd, J=2.80, 8.80 Hz, 1H), 7.38-7.27 (m, 4H), 7.10 (d, J=7.20 Hz, 1H), 4.54-4.49 (m, 1H), 4.12-3.71 (m, 8H), 3.15-2.85 (m, 7H), 2.74 (t, J=6.80 Hz, 2H), 2.68-2.59 (m, 3H), 2.08-1.92 (m, 10H), 1.32-1.21 (m, 2H), 1.03 (t, J=7.20 Hz, 3H).

Similarly, the second eluted fraction was concentrated under reduced pressure at 40° C. and lyophilized to afford Compound 222 (assigned as stereoisomer 2).

Peak-2: 6-[2-cyano-3-[[ethyl(methyl)sulfamoyl]amino]-6-fluoro-phenoxy]-3-[4-[7-[3-(2,4-dioxohexahydropyrimidin-1-yl)-5-fluoro-1-methyl-indazol-6-yl]-2,7-diazaspiro[3.5]nonan-2-yl]cyclohexyl]-4-oxo-quinazoline (Compound 222, 0.045 g, 50.24 mol, 3% yield) as a white solid. UPLC-MS (ES+): m/z 884.3 [M+H]⁺. ¹H NMR (400 MHz, DMSO-d₆): δ 10.56 (s, 1H), 10.09 (bs, 1H), 8.34 (s, 1H), 7.80 (d, J=8.80 Hz, 1H), 7.64 (dd, J=2.80, 8.80 Hz, 1H), 7.47-7.32 (m, 4H), 7.11 (d, J=6.80 Hz, 1H), 4.65-4.55 (m, 1H), 4.32-3.65 (m, 9H), 3.05-2.93 (m, 6H), 2.74 (t, J=6.40 Hz, 2H), 2.72-2.32 (m, 3H), 2.15-1.62 (m, 12H), 1.04 (t, J=7.20 Hz, 3H).

### Synthesis D126: Synthesis of 7-[2-Cyano-3-[[ethyl(methyl)sulfamoyl]amino]-6-fluoro-phenoxy]-2-[[1-[2-[3-(2,4-dioxohexahydropyrimidin-1-yl)-5-fluoro-1-methyl-indazol-6-yl]-2-azaspiro[3.3]heptan-6-yl]-4-piperidyl]methoxy]quinoxaline (Compound 223)

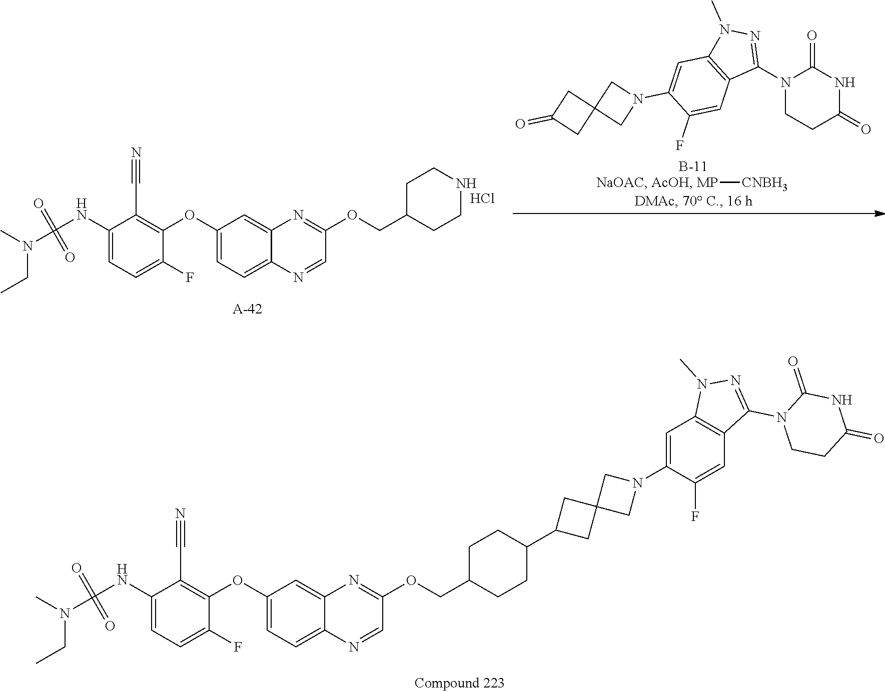

Into a 20 mL pressure vial containing a well-stirred solution of 7-[2-cyano-3-[[ethyl(methyl)sulfamoyl]amino]-6-fluoro-phenoxy]-2-(4-piperidylmethoxy)quinoxaline (A-42) (0.15 g, 272.22 mol, HCl Salt) and 1-[5-fluoro-1-methyl-6-(6-oxo-2-azaspiro[3.3]heptan-2-yl)indazol-3-yl]hexahydropyrimidine-2,4-dione (B-11) (101.09 mg, 272.22 mol) in DMAc (5 mL) was added anhydrous NaOAc (111.65 mg, 1.36 mmol) and resulting mixture was stirred at room temperature for 1 hour. After 1 h, AcOH (816.65 mol, 46.70 μL) was added and stirred at room temperature for 2 hours. Subsequently, MP-CNBH₃ (0.220 g, 272.22 mol) was added and stirred at 70° C. for 16 hours. After completion of reaction, reaction mixture was filtered through sintered funnel and the filtrate was concentrated under reduced pressure to get crude residue which was purified by reverse phase column [Column: Redisep R_{f} Gold® C18-teledyne ISCO, 100 g), Mobile phase A: 10 mM NH₄HCO₃ in milli-Q water; Mobile phase B: acetonitrile]. Fractions were collected and lyophilized to afford 7-[2-cyano-3-[[ethyl(methyl)sulfamoyl]amino]-6-fluoro-phenoxy]-2-[[1-[2-[3-(2,4-dioxohexahydropyrimidin-1-yl)-5-fluoro-1-methyl-indazol-6-yl]-2-azaspiro[3.3]heptan-6-yl]-4-piperidyl]methoxy]quinoxaline (Compound 223, 0.033 g, 36.20 mol, 13% yield) as an off-white solid. UPLC-MS (ES+): m/z 870.3 [M+H]⁺. ¹H NMR (400 MHz, DMSO-d₆): δ 10.51 (s, 1H), 9.48 (bs, 1H), 8.51 (s, 1H), 8.06 (d, J=9.20 Hz, 1H), 7.53-7.40 (m, 2H), 7.33-7.25 (m, 2H), 6.93-6.92 (m, 1H), 6.52 (d, J=7.60 Hz, 1H), 4.34 (d, J=4.40 Hz, 2H), 4.03 (s, 2H), 3.95-3.81 (m, 7H), 3.48-3.39 (m, 2H), 3.29-3.11 (m, 4H), 3.06-3.00 (m, 2H), 2.73 (t, J=6.80 Hz, 2H), 2.61 (s, 3H), 2.48-2.39 (m, 1H), 2.33-2.21 (m, 2H), 2.10-1.85 (m, 3H), 1.59-1.28 (m, 2H), 1.04 (t, J=7.20 Hz, 3H).

### Synthesis D127: Synthesis of 6-[2-cyano-3-[[ethyl(methyl)sulfamoyl]amino]-6-fluoro-phenoxy]-3-[7-[(4R)-4-[3-(2,4-dioxohexahydropyrimidin-1-yl)-5-fluoro-1-methyl-indazol-6-yl]-3,3-difluoro-1-piperidyl]spiro[3.5]nonan-2-yl]-4-oxo-quinazoline (Compound 224)

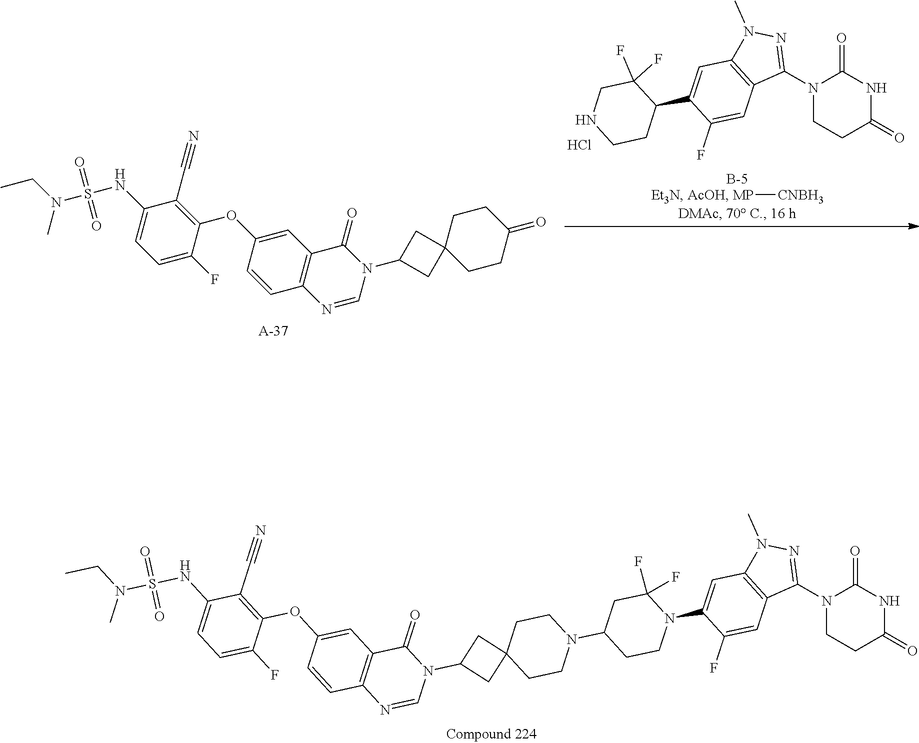

Into a 40 mL pressure glass vial containing a well stirred solution of 6-[2-cyano-3-[[ethyl(methyl)sulfamoyl]amino]-6-fluoro-phenoxy]-4-oxo-3-(7-oxospiro[3.5]nonan-2-yl)quinazoline (A-37) (140 mg, 252.89 mol) and 1-[6-[(4R)-3,3-difluoro-4-piperidyl]-5-fluoro-1-methyl-indazol-3-yl]hexahydropyrimidine-2,4-dione (B-5) (105.66 mg, 252.89 mol, HCl salt) in DMAc (3 mL) was added triethylamine (758.67 mol, 105.74 μL) and stirred at room temperature for 10 min. To the above mixture, AcOH (1.26 mmol, 72.31 μL) was added and stirred at room temperature for 1 hour. Finally, MP-CNBH₃ (120 mg, 289.02 μmol) was added. Resulting mixture was stirred at 70° C. for 16 hours. After completion of the reaction, the reaction mixture was diluted with dichloromethane (10 mL), filtered through cotton pad and filtrate was concentrated under reduced pressure to get a crude residue which was purified by prep HPLC by using Method: [X-BRIDGE C18 (150 mm×19 mm×5 μm) and Mobile phase A:10 mM NH₄HCO₃ (pH:10 adjust with Ammonia) in milli-Q water, Mobile phase B: acetonitrile, flow: 15 mL/min]. Collected fractions were lyophilized to afford 6-[2-cyano-3-[[ethyl(methyl)sulfamoyl]amino]-6-fluoro-phenoxy]-3-[7-[(4R)-4-[3-(2,4-dioxohexahydropyrimidin-1-yl)-5-fluoro-1-methyl-indazol-6-yl]-3,3-difluoro-1-piperidyl]spiro[3.5]nonan-2-yl]-4-oxo-quinazoline (Compound 224, 33 mg, 35.84 mol, 14% yield) as an off-white solid. UPLC-MS (ES+): m/z 919.3 [M+H]⁺. ¹H NMR (400 MHz, DMSO-d₆): δ 10.57 (s, 1H), 10.14 (bs, 1H), 8.40 (s, 1H), 7.83-7.65 (m, 4H), 7.50-7.35 (m, 3H), 4.95-4.87 (m, 1H), 4.02 (s, 3H), 3.92 (t, J=6.40 Hz, 2H), 3.55-3.45 (m, 1H), 3.18-3.13 (m, 4H), 2.78-2.75 (m, 5H), 2.46-2.41 (m, 3H), 2.33-2.18 (m, 4H), 1.95-1.65 (m, 6H), 1.48-1.22 (m, 4H), 1.05 (t, J=3.60 Hz, 3H).

### Synthesis D128: Synthesis of 6-[2-cyano-3-[[ethyl(methyl)sulfamoyl]amino]-6-fluoro-phenoxy]-3-[7-[(4S)-4-[3-(2,4-dioxohexahydropyrimidin-1-yl)-5-fluoro-1-methyl-indazol-6-yl]-3,3-difluoro-1-piperidyl]spiro[3.5]nonan-2-yl]-4-oxo-quinazoline (Compound 225)

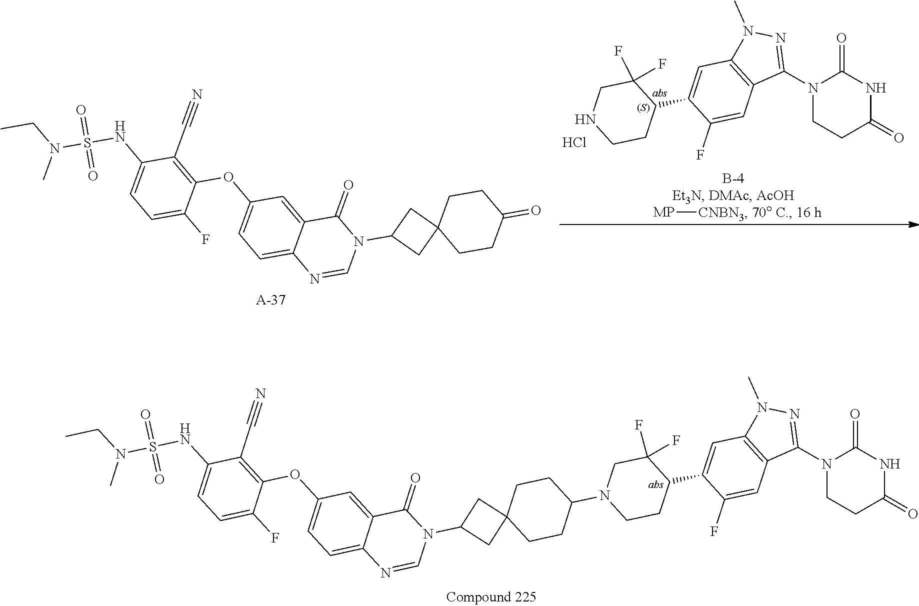

Into a 20 mL pressure vial containing a well-stirred solution of 6-[2-cyano-3-[[ethyl(methyl)sulfamoyl]amino]-6-fluoro-phenoxy]-4-oxo-3-(7-oxospiro[3.5]nonan-2-yl)quinazoline (A-37) (150 mg, 270.95 mol) in DMAc (4 mL) was added triethylamine (1.35 mmol, 188.83 μL) at room temperature under nitrogen atmosphere and stirred for 20 min. Reaction mixture was cooled to 0° C., 1-[6-[(4S)-3,3-difluoro-4-piperidyl]-5-fluoro-1-methyl-indazol-3-yl]hexahydropyrimidine-2,4-dione (B-4) (113.21 mg, 270.95 mol, HCl salt) and AcOH (1.35 mmol, 77.48 μL) were added subsequently and stirred at room temperature for 3 hours. Finally, MP-CNBH₃ (150 mg, 5.61 mmol) was added, and the reaction mixture was stirred at 70° C. for 16 hours. After completion of the reaction, the reaction mixture was filtered and the filtrate was concentrated under reduced pressure to get a crude residue which was purified by preparative HPLC [Column: X-Bridge C18 (250 mm×19 mm×5 m), Mobile phase A: 10 mM NH₄HCO₃ in milli-Q water; Mobile phase B: acetonitrile]. The first eluting fraction was lyophilized to afford 6-[2-cyano-3-[[ethyl(methyl)sulfamoyl]amino]-6-fluoro-phenoxy]-3-[7-[(4S)-4-[3-(2,4-dioxohexahydropyrimidin-1-yl)-5-fluoro-1-methyl-indazol-6-yl]-3,3-difluoro-1-piperidyl]spiro[3.5]nonan-2-yl]-4-oxo-quinazoline (Compound 225, 66 mg, 69.97 mol, 26% yield) as an off-white solid. UPLC-MS (ES+): m/z 919.3 [M+H]⁺. ¹H NMR (400 MHz, DMSO-d₆): δ 10.57 (s, 1H), 10.17 (s, 1H), 8.40 (s, 1H), 7.84-7.66 (m, 4H), 7.50-7.35 (m, 3H), 4.92-4.87 (m, 1H), 4.02 (s, 3H), 3.92 (t, J=6.80 Hz, 2H), 3.63-3.43 (m, 1H), 3.28-3.23 (m, 3H), 3.19-3.13 (m, 1H), 2.79-2.67 (m, 5H), 2.41-2.33 (m, 4H), 2.32-2.13 (m, 4H), 1.95-1.65 (m, 5H), 1.42-1.35 (m, 4H), 1.06 (t, J=7.20 Hz, 3H).

### Synthesis D129: Synthesis of 6-[2-cyano-3-[[ethyl(methyl)sulfamoyl]amino]-6-fluoro-phenoxy]-3-[7-[[1-[3-(2,4-dioxohexahydropyrimidin-1-yl)-5-fluoro-1-methyl-indazol-6-yl]-4-hydroxy-4-piperidyl]methyl]-7-azaspiro[3.5]nonan-2-yl]-4-oxo-quinazoline (Compound 226)

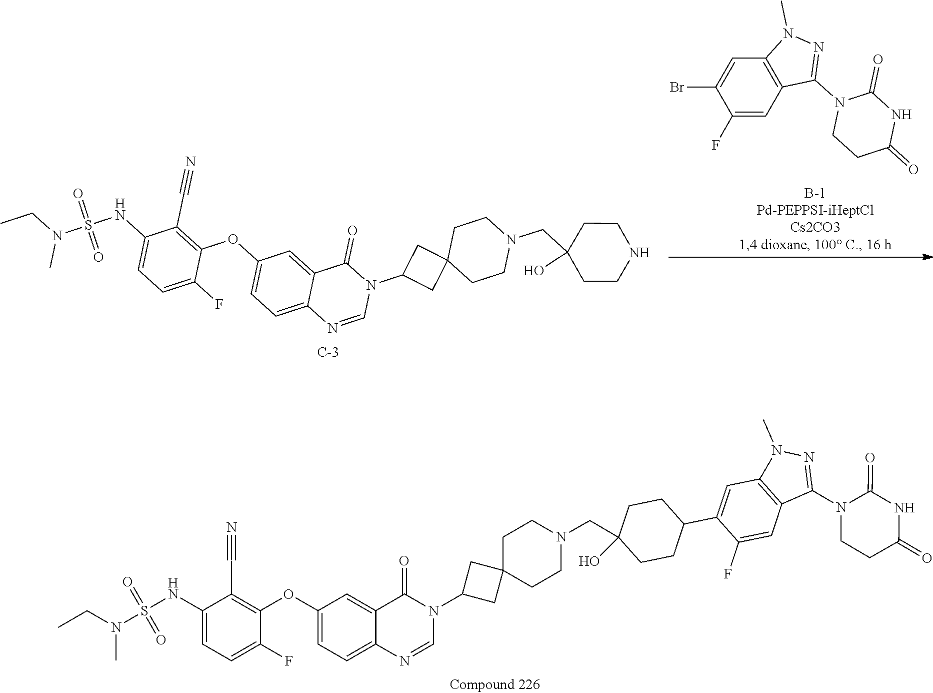

Into a 10 mL pressure vial containing a well stirred solution of 6-[2-cyano-3-[[ethyl(methyl)sulfamoyl]amino]-6-fluoro-phenoxy]-3-[7-[(4-hydroxy-4-piperidyl)methyl]-7-azaspiro[3.5]nonan-2-yl]-4-oxo-quinazoline (C-3, 150 mg, 202.82 mol) in 1,4-dioxane (5 mL) were added 1-(6-bromo-5-fluoro-1-methyl-indazol-3-yl)hexahydropyrimidine-2,4-dione (B-1) (76.88 mg, 223.11 mol) and Cs₂CO₃ (198.25 mg, 608.48 mol) at room temperature under nitrogen atmosphere. The resulting mixture was purged with nitrogen gas for 10 min. Pd-peppsi-ihept Cl (19.73 mg, 20.28 mol) was added to the reaction mixture and stirred at 100° C. for 16 hours. Reaction mixture was allowed to room temperature, filtered over celite bed and washed with dichloromethane (20 mL). The filtrate was concentrated under reduced pressure to get a crude residue, which was purified by prep HPLC [Column: X-Bridge C8 (250 mm*19 mm*5 μm), Mobile phase A: 10 mM NH₄HCO₃ & NH₄OH (pH 10) in milli-Q water; Mobile phase B: acetonitrile] to afford 6-[2-cyano-3-[[ethyl(methyl)sulfamoyl]amino]-6-fluoro-phenoxy]-3-[7-[[1-[3-(2,4-dioxohexahydropyrimidin-1-yl)-5-fluoro-1-methyl-indazol-6-yl]-4-hydroxy-4-piperidyl]methyl]-7-azaspiro[3.5]nonan-2-yl]-4-oxo-quinazoline (Compound 226, 3.0 mg, 3.26 mol, 2% yield) as an off-white solid. UPLC-MS (ES+): m/z 914.3 [M+H]⁺. ¹H NMR (DMSO-d₆): δ 10.52 (s, 1H), 8.38 (s, 1H), 7.75 (d, J=8.80 Hz, 1H), 7.61 (dd, J=2.80, 8.80 Hz, 1H), 7.25-7.32 (m, 3H), 7.12 (d, J=7.20 Hz, 1H), 6.52 (s, 1H), 4.95-4.90 (m, 1H), 3.95-3.88 (m, 4H), 3.29-3.28 (m, 2H), 3.18-2.95 (m, 6H), 2.74 (t, J=6.40 Hz, 2H), 2.61-2.55 (m, 6H), 2.48-2.41 (m, 5H), 2.25-2.19 (m, 3H), 1.85-1.51 (m, 7H), 1.03 (t, J=7.20 Hz, 3H).

### Synthesis D130: Synthesis of 6-[2-cyano-3-[[ethyl(methyl)sulfamoyl]amino]-6-fluoro-phenoxy]-3-[2-[(4S)-4-[3-(2,4-dioxohexahydropyrimidin-1-yl)-5-fluoro-1-methyl-indazol-6-yl]-3,3-difluoro-1-piperidyl]spiro[3.3]heptan-6-yl]-4-oxo-quinazoline (Compound 227)

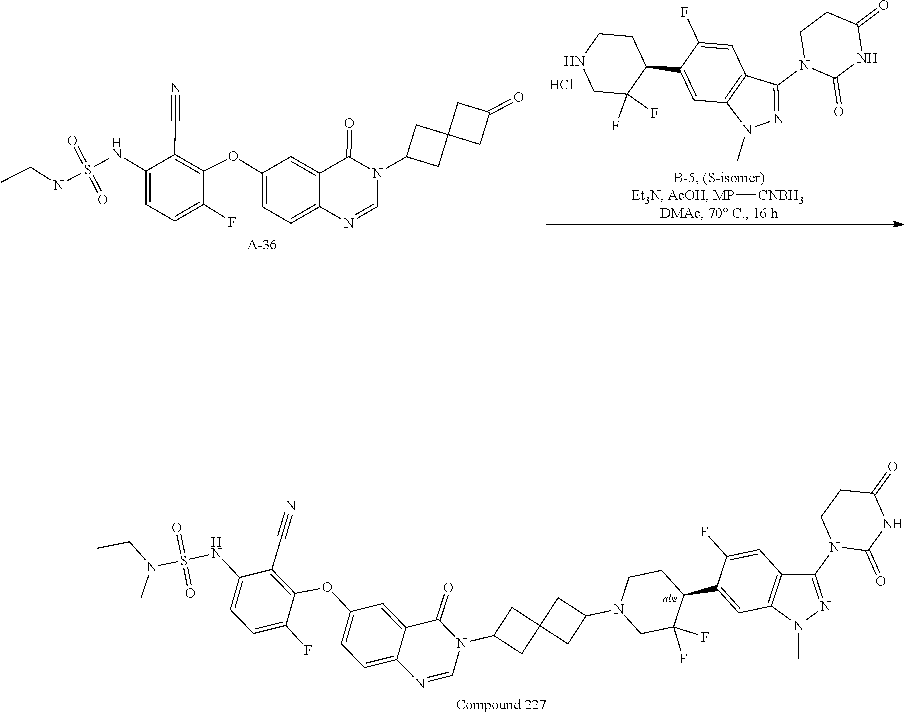

Into a 8 mL pressure vial containing a well-stirred solution of 1-[6-[(4S)-3,3-difluoro-4-piperidyl]-5-fluoro-1-methyl-indazol-3-yl]hexahydropyrimidine-2,4-dione (B-5) (87.45 mg, 209.30 mol, HCl salt) in DMAc (5 mL) was added triethylamine (627.91 mol, 87.52 μL) at room temperature under nitrogen atmosphere and mixture was stirred for 20 min. Reaction mixture was cooled to 0° C. and was added 6-[2-cyano-3-[[ethyl(methyl)sulfamoyl]amino]-6-fluoro-phenoxy]-4-oxo-3-(2-oxospiro[3.3]heptan-6-yl)quinazoline (A-36) (255.81 mg, 209.30 mol) followed by AcOH (1.05 mmol, 59.84 μL). The resulting mixture was stirred for 3 h at room temperature. Finally, MP-CNBH₃ (110 mg, 209.30 μmol) was added, and the reaction mixture was stirred at 70° C. for 16 hours. After completion of the reaction, the reaction mixture was filtered and the filtrate was concentrated under reduced pressure to get a crude residue which was purified by preparative HPLC [Column: X-Bridge C18 (150 mm*19 mm*5 m), Mobile phase A: 10 mm NH₄HCO₃ in milli-Q water; Mobile phase B: acetonitrile]. The eluting fraction was concentrated under reduced pressure at 40° C. and lyophilized to afford 6-[2-cyano-3-[[ethyl(methyl)sulfamoyl]amino]-6-fluoro-phenoxy]-3-[2-[(4S)-4-[3-(2,4-dioxohexahydropyrimidin-1-yl)-5-fluoro-1-methyl-indazol-6-yl]-3,3-difluoro-1-piperidyl]spiro[3.3]heptan-6-yl]-4-oxo-quinazoline (Compound 227, 29.32 mg, 32.40 mol, 15% yield) as a white solid. UPLC-MS (ES+): m/z 891.3 [M+H]⁺. ¹H NMR (400 MHz, DMSO-d₆): δ 10.57 (s, 1H), 10.18 (s, 1H), 8.37 (d, J=0.8 Hz, 1H), 8.00-7.71 (m, 3H), 7.70-7.62 (m, 1H), 7.51-7.40 (m, 2H), 7.34 (d, J=2.8 Hz, 1H), 4.90-4.69 (m, 1H), 4.02 (s, 3H), 3.92 (t, J=6.8 Hz, 2H), 3.63-3.45 (m, 1H), 3.21-3.02 (m, 3H), 3.01-2.82 (m, 2H), 2.81-2.71 (m, 5H), 2.64-2.53 (m, 2H), 2.49-2.38 (m, 4H), 2.31-2.23 (m, 1H), 2.20-2.03 (m, 2H), 2.02-1.80 (m, 3H), 1.05 (t, J=6.8 Hz, 3H).

### Synthesis D131: Synthesis of 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-fluoro-4-piperidyl]methoxy]quinoxaline (Compound 228 and Compound 229)

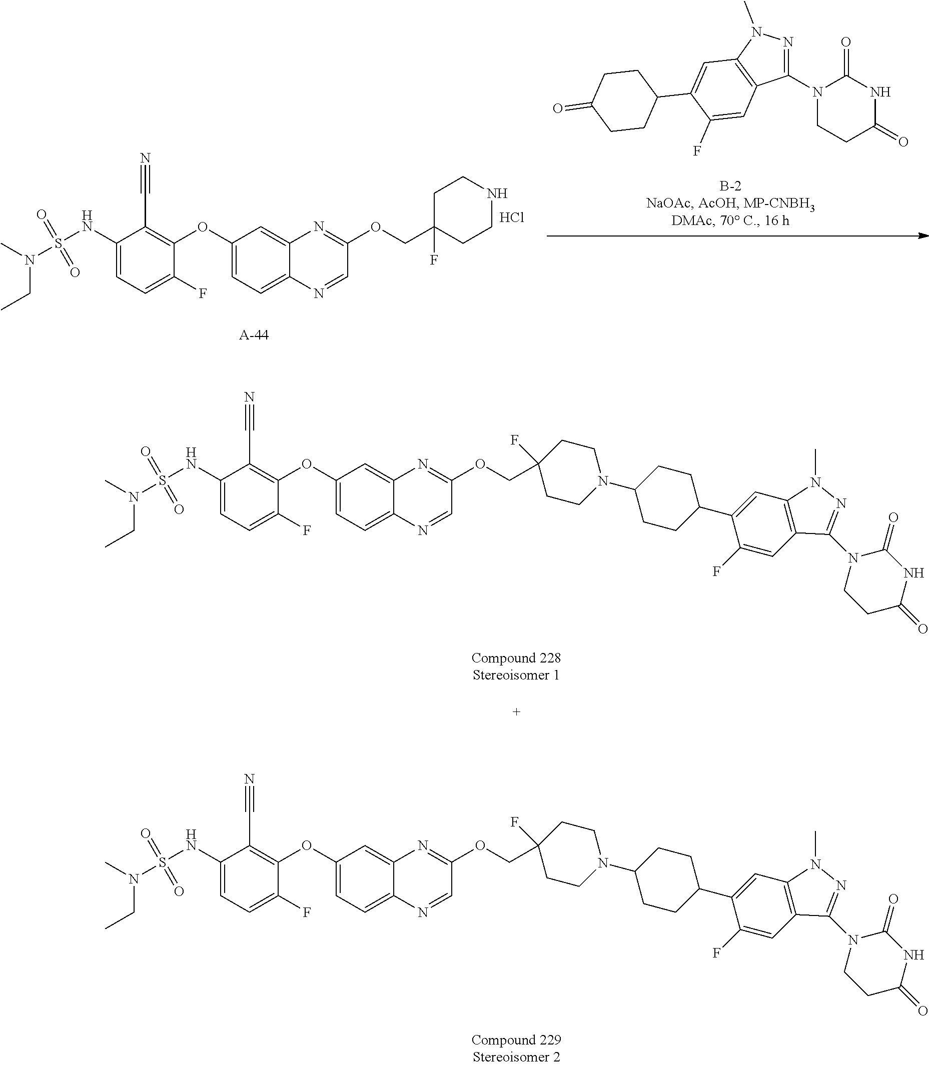

Into a 40 mL glass vial with pressure released cap containing a well-stirred solution of 7-[2-cyano-3-[[ethyl(methyl)sulfamoyl]amino]-6-fluoro-phenoxy]-2-[(4-fluoro-4-piperidyl)methoxy]quinoxaline (A-44) (270 mg, 403.32 mol, HCl salt) in DMAc (5 mL) was added NaOAc (165.42 mg, 2.02 mmol) at room temperature and stirred at room temperature for 5 min. Subsequently, AcOH (1.21 mmol, 69.20 μL) and 1-[5-fluoro-1-methyl-6-(4-oxocyclohexyl)indazol-3-yl]hexahydropyrimidine-2,4-dione (B-2) (216.81 mg, 604.98 μmol) were added and stirred at room temperature for 2 hours. Finally, MP-CNBH₃ (270 mg) was added to the reaction mixture and stirred at 70° C. for 16 hours. After completion of the reaction, the reaction mixture was filtered and filtrate was concentrated under reduced pressure to get crude residue which was purified by preparative HPLC [Column: X-Bridge C8 (250 mm*19 mm*5 μm), Mobile phase A: 10 mM NH₄HCO₃ & NH₄OH_pH-10 in milli-Q water; Mobile phase B: acetonitrile]. The first eluting fraction (Peak-1) was lyophilized to afford Compound 228 (assigned as stereoisomer 1).

Peak-1: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-fluoro-4-piperidyl]methoxy]quinoxaline (Compound 228, 27 mg, 30.80 mol, 8% yield) as a white solid. UPLC-MS (ES+): m/z 875.3 [M+H]⁺. ¹H NMR (400 MHz, DMSO-d₆): δ 10.55 (s, 1H), 9.58 (bs, 1H), 8.60 (s, 1H), 8.09 (d, J=9.20 Hz, 1H), 7.56-7.54 (m, 3H), 7.39-7.35 (m, 2H), 6.96 (s, 1H), 4.64-4.59 (m, 2H), 4.00 (s, 3H), 3.91 (t, J=6.40 Hz, 2H), 3.12-3.02 (m, 4H), 2.89-2.77 (m, 3H), 2.75 (t, J=6.40 Hz, 2H), 2.68-2.64 (m, 3H), 2.24-1.95 (m, 8H), 1.69-1.58 (m, 4H), 1.05 (t, J=7.20 Hz, 3H).

Similarly, the second eluted fraction (Peak-2) was lyophilized to afford Compound 229 (assigned as stereoisomer 2).

Peak-2: 7-[2-cyano-3-[[ethyl(methyl)sulfamoyl]amino]-6-fluoro-phenoxy]-2-[[1-[4-[3-(2,4-dioxohexahydropyrimidin-1-yl)-5-fluoro-1-methyl-indazol-6-yl]cyclohexyl]-4-fluoro-4-piperidyl]methoxy]quinoxaline (Compound 229, 24 mg, 27.36 mol, 7% yield) as a white solid. UPLC-MS (ES+): m/z 875.3 [M+H]⁺. ¹H NMR (400 MHz, DMSO-d₆): δ 10.56 (s, 1H), 10.19 (bs, 1H), 8.60 (s, 1H), 8.09 (d, J=8.80 Hz, 1H), 7.65-7.25 (m, 5H), 6.98 (s, 1H), 4.63-4.58 (m, 2H), 4.02 (s, 3H), 3.91 (t, J=6.80 Hz, 2H), 3.15-2.96 (m, 5H), 2.76 (t, J=6.80 Hz, 2H), 2.68-2.67 (m, 3H), 2.34-1.70 (m, 15H), 1.05 (t, J=7.20 Hz, 3H).

### Synthesis D132: Synthesis of (3R)-3-[6-[2-cyano-3-[[ethyl(methyl)sulfamoyl]amino]-6-fluoro-phenoxy]-4-oxo-quinazolin-3-yl]-8-[[1-[3-(2,4-dioxohexahydropyrimidin-1-yl)-5-fluoro-1-methyl-indazol-6-yl]-4-hydroxy-4-piperidyl]methyl]-1-oxa-8-azaspiro[4.5]decane (Compound 230)

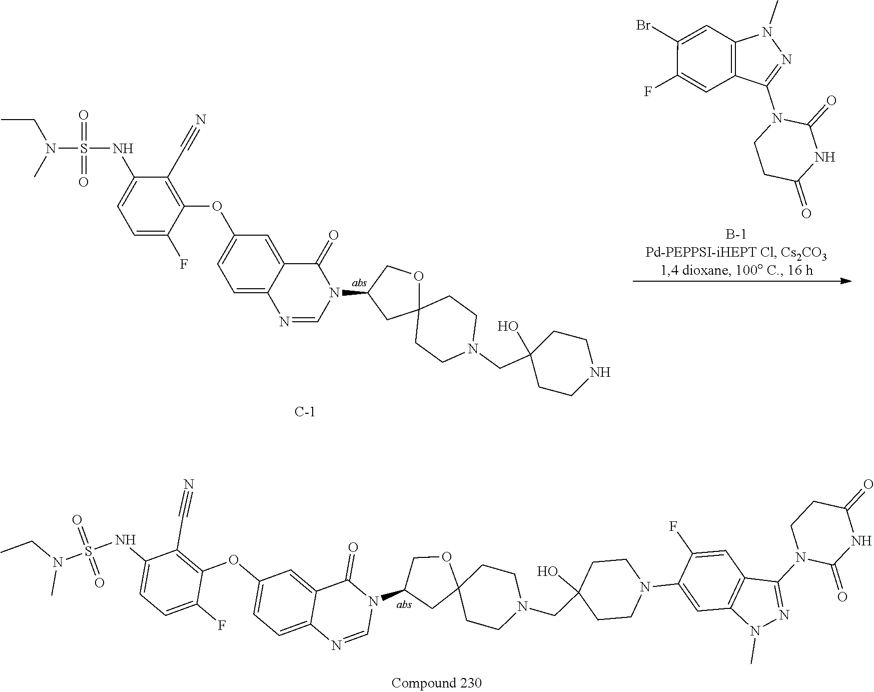

Into a 30 mL pressure vial containing a well stirred solution of (3R)-3-[6-[2-cyano-3-[[ethyl(methyl)sulfamoyl]amino]-6-fluoro-phenoxy]-4-oxo-quinazolin-3-yl]-8-[(4-hydroxy-4-piperidyl)methyl]-1-oxa-8-azaspiro[4.5]decane (C-1, 130 mg, 148.89 mol) in 1,4-dioxane (10 mL) were added 1-(6-bromo-5-fluoro-1-methyl-indazol-3-yl)hexahydropyrimidine-2,4-dione (B-1) (61.57 mg, 178.67 mol) and Cs₂CO₃ (145.54 mg, 446.68 mol) at room temperature under nitrogen atmosphere. The resulting mixture was purged with nitrogen gas for 10 min. Finally, Pd-peppsi-ihept Cl (14.47 mg, 14.89 mol) was added to the reaction mixture and stirred at 100° C. for 16 hours. After completion of the reaction, the reaction mixture was allowed to room temperature, filtered over celite bed, and washed with 5% methanol in dichloromethane (20 mL). The filtrate was concentrated under reduced pressure to get the crude compound, which was purified by reverse phase column chromatography (Column: Redisep, C18 120 g, mobile phase A: 0.1% NH₄HCO₃ in water, mobile phase B: acetonitrile, flow rate: 15 mL/min) and pure fraction was lyophilized to afford (3R)-3-[6-[2-cyano-3-[[ethyl(methyl)sulfamoyl]amino]-6-fluoro-phenoxy]-4-oxo-quinazolin-3-yl]-8-[[1-[3-(2,4-dioxohexahydropyrimidin-1-yl)-5-fluoro-1-methyl-indazol-6-yl]-4-hydroxy-4-piperidyl]methyl]-1-oxa-8-azaspiro[4.5]decane (Compound 230, 26 mg, 26.95 mol, 18% yield) as an off-white solid. UPLC-MS (ES+): m/z 930.3 [M+H]⁺. H NMR (DMSO-d₆): δ 10.53 (s, 1H), 8.33 (s, 1H), 7.77 (d, J=8.80 Hz, 1H), 7.65 (dd, J=2.80, 8.80 Hz, 1H), 7.55-7.42 (m, 1H), 7.35-7.33 (m, 3H), 7.13 (d, J=7.20 Hz, 1H), 5.29 (s, 1H), 4.13-4.10 (m, 3H), 3.95 (bs, 3H), 3.90 (t, J=6.80 Hz, 2H), 3.21-2.98 (m, 9H), 2.74 (t, J=6.80 Hz, 2H), 2.68-2.51 (m, 3H), 2.45-2.39 (m, 2H), 2.10-1.61 (m, 11H), 1.04 (t, J=7.20 Hz, 3H).

### Synthesis D133: Synthesis of (3R)-3-[6-[2-cyano-3-[[ethyl(methyl)sulfamoyl]amino]-6-fluoro-phenoxy]-4-oxo-quinazolin-3-yl]-8-[[1-[3-(2,4-dioxohexahydropyrimidin-1-yl)-5-fluoro-1-methyl-indazol-6-yl]azetidin-3-yl]methyl]-1-oxa-8-azaspiro[4.5]decane (Compound 231)

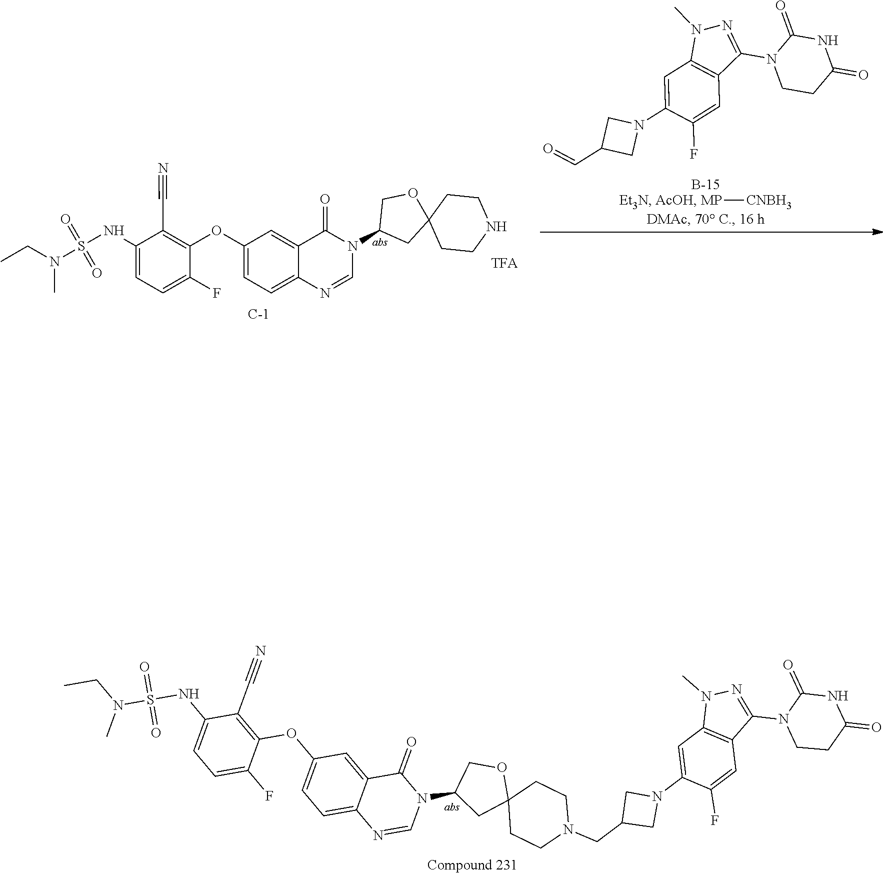

Into a 40 mL glass pressure capped vial containing a well-stirred solution of (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (C-1) (200.00 mg, 259.46 mol, TFA salt) in DMAc (5 mL) was added triethylamine, 99% (778.37 mol, 108.49 μL) at room temperature and stirred for 15 min. Subsequently, 1-[3-(2,4-dioxohexahydropyrimidin-1-yl)-5-fluoro-1-methyl-indazol-6-yl]azetidine-3-carbaldehyde (B-15) (268.10 mg, 518.91 mol) and AcOH (518.91 mol, 29.67 L) were added at room temperature and the mixture was stirred for 2 hours. Finally, MP-CNBH₃ (200 mg, 259.46 mol) was added, and the resulting mixture was stirred at 70° C. for 16 hours. After completion of the reaction, the reaction mixture was diluted with dichloromethane (10 mL), filtered through a cotton pad and the filtrate was concentrated under reduced pressure to get a crude residue, which was purified by reverse phase column chromatography [Column: Redisep R_{f} Gold® reversed-phase C18-teledyne ISCO, 120 g, mobile phase A: 10 mM NH₄HCO₃ in milli-Q water; mobile phase B: acetonitrile]. The pure fractions were combined and lyophilized to afford desired product with 96% purity, and it was showing aliphatic impurities in NMR. Further repurified by preparative HPLC (Column: X-Bridge C18 (150 mm×19 mm×5 μm), mobile Phase A: 10 mM NH₄HCO₃ in milli-Q water; mobile phase B: acetonitrile]. The pure fractions were lyophilized to afford (3R)-3-[6-[2-cyano-3-[[ethyl(methyl)sulfamoyl]amino]-6-fluoro-phenoxy]-4-oxo-quinazolin-3-yl]-8-[[1-[3-(2,4-dioxohexahydropyrimidin-1-yl)-5-fluoro-1-methyl-indazol-6-yl]azetidin-3-yl]methyl]-1-oxa-8-azaspiro[4.5]decane (Compound 231, 31 mg, 34.71 mol, 13% yield) as an off-white solid. UPLC-MS (ES+): m/z 886.2 [M+H]⁺. ¹H NMR (400 MHz, DMSO-d₆): δ 10.51 (s, 1H), 9.67 (bs, 1H), 8.33 (s, 1H), 7.78 (d, J=8.80 Hz, 1H), 7.66 (dd, J=3.20, 9.00 Hz, 2H), 7.38-7.35 (m, 2H), 7.28 (d, J=12.80 Hz, 1H), 6.54 (d, J=7.60 Hz, 1H), 5.30-5.27 (m, 1H), 4.19-4.09 (m, 4H), 3.90-3.87 (m, 5H), 3.75-3.66 (m, 2H), 3.09-2.84 (m, 8H), 2.75-2.67 (m, 6H), 2.42-2.36 (m, 1H), 2.13-2.08 (m, 1H), 1.90-1.76 (m, 4H), 1.04 (t, J=7.20 Hz, 3H).

### Synthesis D134: Synthesis of 6-[2-Cyano-3-[[ethyl(methyl)sulfamoyl]amino]-6-fluoro-phenoxy]-4-[[2-[4-[3-(2,4-dioxohexahydropyrimidin-1-yl)-5-fluoro-1-methyl-indazol-6-yl]cyclohexyl]-2-azaspiro[3.3]heptan-6-yl]oxy]quinazoline (Compound 232 and Compound 233)

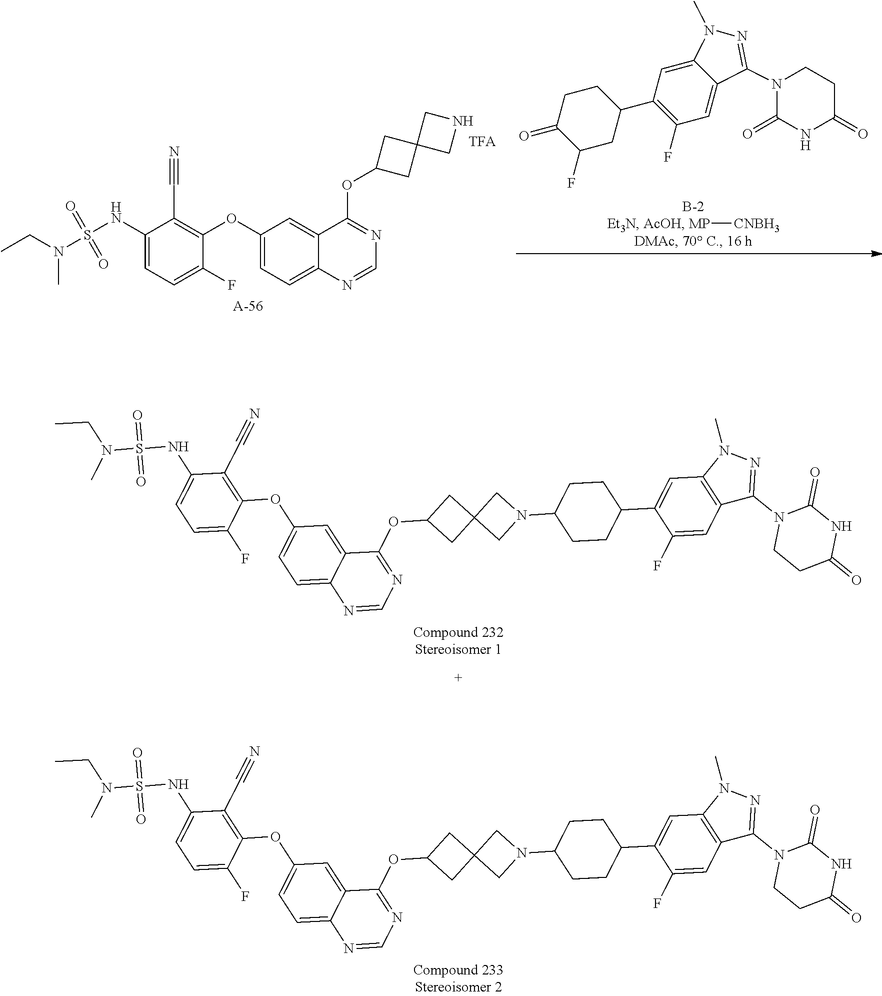

Into a 40 mL pressure vial containing a well-stirred solution of 4-(2-azaspiro[3.3]heptan-6-yloxy)-6-[2-cyano-3-[[ethyl(methyl)sulfamoyl]amino]-6-fluoro-phenoxy]quinazoline (A-56) (400 mg, 622.43 mol, TFA salt) in DMAc (4 mL) was added triethylamine (3.11 mmol, 433.77 L) at room temperature and stirred for 5 min. Subsequently, 1-[5-fluoro-1-methyl-6-(4-oxocyclohexyl)indazol-3-yl]hexahydropyrimidine-2,4-dione (B-2) (223.06 mg, 622.43 mol) and AcOH (657.05 mol, 37.58 μL) were added and stirred for 2 h at room temperature. Finally, MP-CNBH₃ (500 mg, 622.43 mol) was added and resulting reaction mixture was stirred at 70° C. for 16 hours. After completion of the reaction, the reaction mixture was filtered through cotton pad and filtrate was concentrated under reduced pressure to get a crude residue which was purified by preparative HPLC [Column: X-Bridge C18 (150 mm*19 mm*5 m), Mobile phase A: 10 mM NH₄HCO₃ (pH adjusted to 10 with NH₄OH) in milli-Q water; Mobile phase B: acetonitrile]. The first eluting fraction (Peak-1) was lyophilized to afford Compound 232 (assigned as stereoisomer 1).

Peak-1: 6-[2-cyano-3-[[ethyl(methyl)sulfamoyl]amino]-6-fluoro-phenoxy]-4-[[2-[4-[3-(2,4-dioxohexahydropyrimidin-1-yl)-5-fluoro-1-methyl-indazol-6-yl]cyclohexyl]-2-azaspiro[3.3]heptan-6-yl]oxy]quinazoline (Compound 232, 38 mg, 44.03 mol, 7% yield) as an off-white solid. UPLC-MS (ES+): m/z 855.3 [M+H]⁺. ¹H NMR (400 MHz, DMSO-d₆): δ 10.55 (s, 1H), 9.82 (bs, 1H), 8.73 (s, 1H), 7.99 (d, J=9.20 Hz, 1H), 7.78 (dd, J=2.80, 9.20 Hz, 1H), 7.53-7.51 (d, J=5.6 Hz, 1H), 7.38 (d, J=10.80 Hz, 2H), 7.30-7.25 (m, 2H), 5.38-5.28 (m, 1H), 4.40-3.88 (m, 9H), 3.48-3.40 (m, 1H), 3.25-3.09 (m, 1H), 3.05-2.96 (m, 2H), 2.90-2.80 (m, 2H), 2.75 (t, J=6.80 Hz, 2H), 2.60-2.57 (m, 4H), 2.42-2.39 (m, 1H), 2.10-1.91 (m, 4H), 1.66-1.56 (m, 2H), 1.35-1.20 (m, 2H), 1.03 (t, J=7.20 Hz, 3H)

Similarly, the second eluting fraction (Peak-2) was lyophilized to afford Compound 233 (assigned as stereoisomer 2).

Peak-2: 6-[2-cyano-3-[[ethyl(methyl)sulfamoyl]amino]-6-fluoro-phenoxy]-4-[[2-[4-[3-(2,4-dioxohexahydropyrimidin-1-yl)-5-fluoro-1-methyl-indazol-6-yl]cyclohexyl]-2-azaspiro[3.3]heptan-6-yl]oxy]quinazoline (Compound 233, 58 mg, 67.37 mol, 11% yield) as an off-white solid. UPLC-MS (ES+): m/z 855.3 [M+H]⁺. ¹H NMR (400 MHz, DMSO-d₆): δ 10.56 (s, 1H), 9.51 (bs, 1H), 8.73 (s, 1H), 7.99 (d, J=9.20 Hz, 1H), 7.77 (dd, J=2.80, 9.20 Hz, 1H), 7.53-7.30 (m, 5H), 5.38-5.31 (m, 1H), 4.50-4.10 (m, 3H), 3.99 (s, 3H), 3.90 (t, J=6.80 Hz, 2H), 3.65-3.40 (m, 1H), 3.02-2.88 (m, 4H), 2.85-2.71 (m, 4H), 2.65-2.58 (m, 4H), 1.91-1.50 (m, 8H), 1.03 (t, J=7.20 Hz, 3H).

### Synthesis D135: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]cyclopropanesulfonamide (Compound 234)

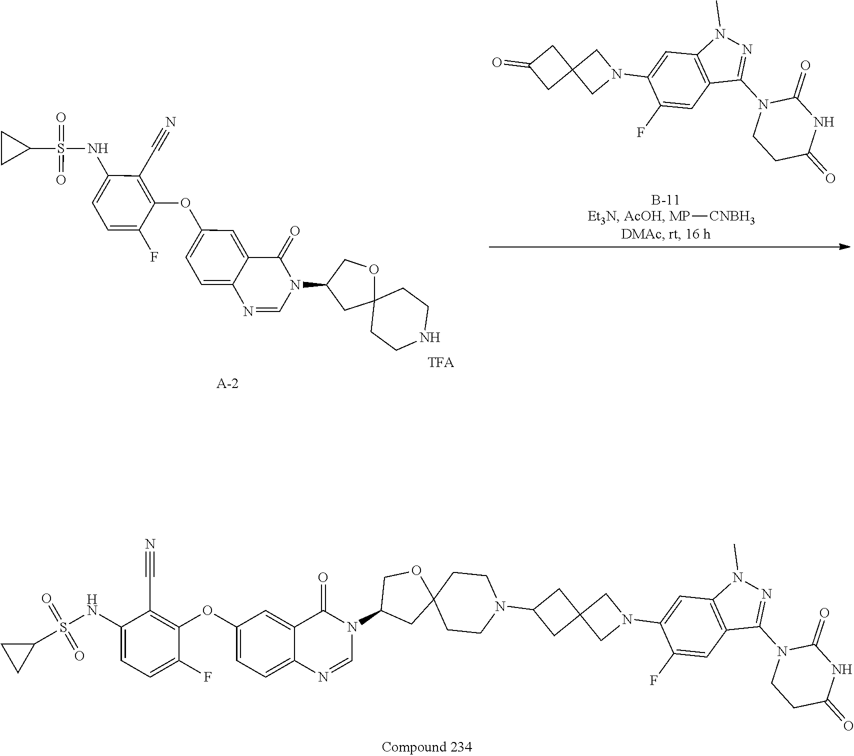

Into a 20 mL reaction vial containing a solution of N-[2-cyano-4-fluoro-3-[3-[(3R)-1-oxa-8-azaspiro[4.5]decan-3-yl]-4-oxo-quinazolin-6-yl]oxy-phenyl]cyclopropanesulfonamide (A-2) (200 mg, 306.00 mol, TFA salt) in DMAc (5 mL) was added triethylamine (611.99 mol, 85.30 L) at room temperature and stirred for 10 min. Subsequently, AcOH (917.99 mol, 52.5 μL) and 1-[5-fluoro-1-methyl-6-(6-oxo-2-azaspiro[3.3]heptan-2-yl)indazol-3-yl]hexahydropyrimidine-2,4-dione (B-11) (136.36 mg, 367.20 mol) were added and stirred at the room temperature for 2 hours. Finally, MP-CNBH₃ (100 mg, 306.00 mol) was added and stirred for 16 h at room temperature. After completion of the reaction, the reaction mixture was filtered through Celite pad and the filtrate was concentrated. The crude was purified by reverse phase column chromatography (Column: Redisep R_{f} Gold® reversed-phase C18-teledyne ISCO, 60 g, Mobile phase A: 10 mM NH₄HCO₃ in mill-Q water; Mobile phase B: acetonitrile). The eluting compound was collected and lyophilized to get 45 mg of compound as an off-white solid, which is impure. The resultant compound was further purified by preparative HPLC [Column: X BRIDGE C18 (250 mm×19 mm×5 μm), Mobile phase A: 10 mM NH₄HCO₃ (pH:10 adjusted with ammonia) in milli-Q water; Mobile phase B: acetonitrile), Flow rate: 14 mL/min]. The eluting compound was collected and lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]cyclopropanesulfonamide (Compound 234, 20.86 mg, 22.96 mol, 8% yield) as an off-white solid. UPLC-MS (ES+): m/z 895.2 [M+H]⁺. ¹H NMR (400 MHz, DMSO-d₆): δ 10.51 (s, 1H), 9.80 (s, 1H), 8.32 (s, 1H), 7.77 (d, J=8.80 Hz, 1H), 7.66 (dd, J=2.80, 8.80 Hz, 1H), 7.53 (s, 1H), 7.37 (d, J=3.20 Hz, 2H), 7.27 (d, J=12.80 Hz, 1H), 6.51 (d, J=7.60 Hz, 1H), 5.31-5.26 (m, 1H), 4.18-4.08 (m, 2H), 4.02 (s, 2H), 3.92-3.86 (m, 7H), 3.20-2.80 (m, 2H), 2.75-2.67 (m, 3H), 2.65-2.50 (m, 3H), 2.48-2.33 (m, 3H), 2.30-2.18 (m, 2H), 2.15-2.05 (m, 1H), 2.00-1.55 (m, 4H), 0.85 (d, J=4.40 Hz, 4H).

### Synthesis D136: Synthesis of (7R)-7-[6-[2-cyano-3-[[ethyl(methyl)sulfamoyl]amino]-6-fluoro-phenoxy]-4-oxo-quinazolin-3-yl]-2-[4-[3-(2,4-dioxohexahydropyrimidin-1-yl)-5-fluoro-1-methyl-indazol-6-yl]cyclohexyl]-5-oxa-2-azaspiro[3.4]octane (Compound 235 and Compound 236)

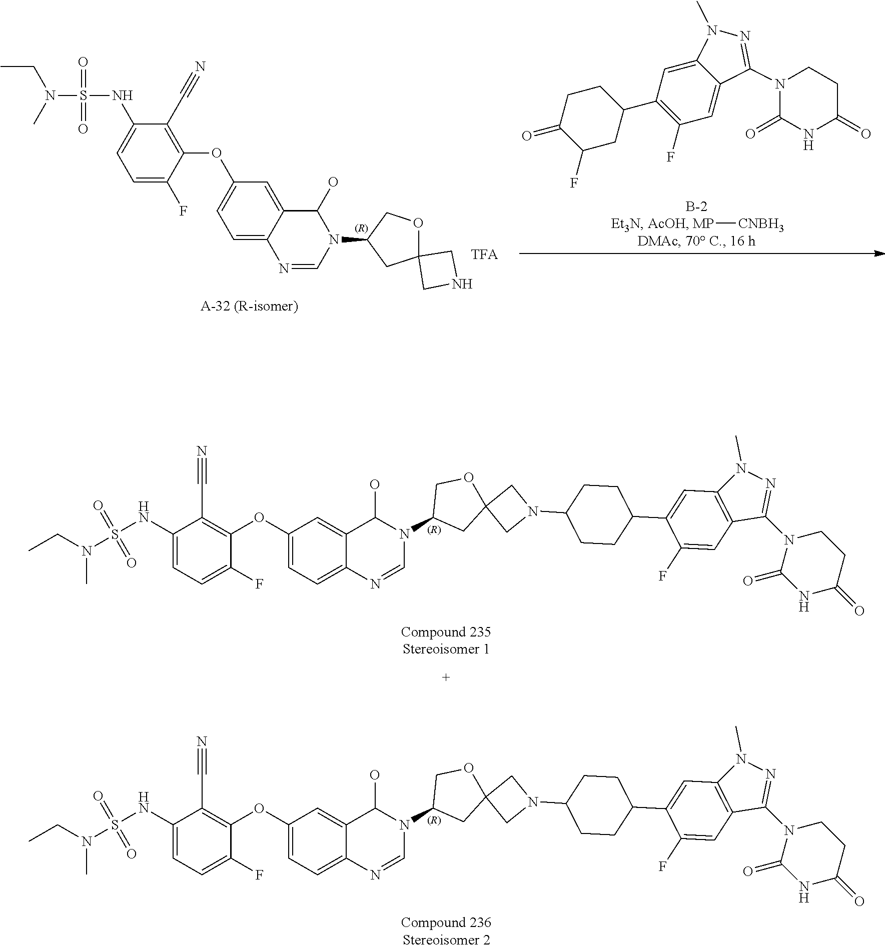

Into a 20 mL pressure vial containing a well stirred solution of (7R)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-32) (500 mg, 778.12 mol, TFA salt) in DMAc (10.00 mL) was added triethylamine (3.89 mmol, 542.27 μL) at room temperature and the resulting mixture was stirred for 5 min. Subsequently, AcOH (778.12 mol, 44.50 μL) and 1-[5-fluoro-1-methyl-6-(4-oxocyclohexyl)indazol-3-yl]hexahydropyrimidine-2,4-dione (B-2) (334.62 mg, 933.74 μmol) was added and stirred for 1 hour. Finally, MP-CNBH₃ (500 mg, 778.12 μmol) was added and resulting reaction mixture was stirred at 70° C. for 16 hours. After completion of the reaction, the reaction mixture was filtered through cotton pad and the filtrate was concentrated under reduced pressure to get a crude residue which was purified by preparative HPLC [Column: X-BRIDGE C18 (250 mm*19 mm*5 μm), Mobile phase A: NH₄HCO₃ in milli-Q water; Mobile phase B: acetonitrile, flow rate 15 mL/min]. The first eluting fraction (Peak-1) was lyophilized to afford Compound 235 (assigned as stereoisomer 1).

Peak-1: (7R)-7-[6-[2-cyano-3-[[ethyl(methyl)sulfamoyl]amino]-6-fluoro-phenoxy]-4-oxo-quinazolin-3-yl]-2-[4-[3-(2,4-dioxohexahydropyrimidin-1-yl)-5-fluoro-1-methyl-indazol-6-yl]cyclohexyl]-5-oxa-2-azaspiro[3.4]octane (Compound 235.27 mg, 30.57 mol, 4% yield) as an off-white solid. UPLC-MS (ES+): m/z 871.3 [M+H]⁺. ¹H NMR (400 MHz, DMSO-d₆): δ 10.56 (s, 1H), 10.08 (bs, 1H), 8.21 (s, 1H), 7.78 (d, J=8.80 Hz, 1H), 7.69 (dd, J=3.20, 8.80 Hz, 1H), 7.53-7.41 (m, 2H), 7.38 (d, J=10.80, Hz, 1H), 7.32-7.29 (m, 2H), 5.20-5.10 (m, 1H), 4.40-3.87 (m, 11H), 3.05-2.98 (m, 3H), 2.87-2.79 (m, 1H), 2.76 (t, J=6.80 Hz, 2H), 2.65-2.55 (m, 5H), 2.10-1.89 (m, 4H), 1.66-1.56 (m, 2H), 1.40-1.25 (m, 2H), 1.03 (t, J=7.20 Hz, 3H).

Similarly, the second eluted fraction (Peak-2) was lyophilized to afford Compound 236 (assigned as stereoisomer 2).

Peak-2: (7R)-7-[6-[2-cyano-3-[[ethyl(methyl)sulfamoyl]amino]-6-fluoro-phenoxy]-4-oxo-quinazolin-3-yl]-2-[4-[3-(2,4-dioxohexahydropyrimidin-1-yl)-5-fluoro-1-methyl-indazol-6-yl]cyclohexyl]-5-oxa-2-azaspiro[3.4]octane (Compound 236, 22 mg, 24.92 mol, 3% yield) as an off-white solid. UPLC-MS (ES+): m/z 871.3 [M+H]⁺. ¹H NMR (400 MHz, DMSO-d₆): δ 10.56 (s, 1H), 10.08 (bs, 1H), 8.21 (s, 1H), 7.78 (d, J=8.80 Hz, 1H), 7.69 (dd, J=3.20, 8.80 Hz, 1H), 7.60-7.20 (m, 5H), 5.35-5.25 (m, 1H), 4.70-3.88 (m, 9H), 3.65-3.40 (m, 1H), 3.18-2.85 (m, 4H), 2.76 (t, J=6.80 Hz, 2H), 2.64 (s, 3H), 2.00-1.50 (m, 8H), 1.03 (t, J=6.80 Hz, 3H).

### Synthesis D137: Synthesis of (7S)-7-[6-[2-cyano-3-[[ethyl(methyl)sulfamoyl]amino]-6-fluoro-phenoxy]-4-oxo-quinazolin-3-yl]-2-[4-[3-(2,4-dioxohexahydropyrimidin-1-yl)-5-fluoro-1-methyl-indazol-6-yl]cyclohexyl]-5-oxa-2-azaspiro[3.4]octane (Compound 237 and Compound 238)

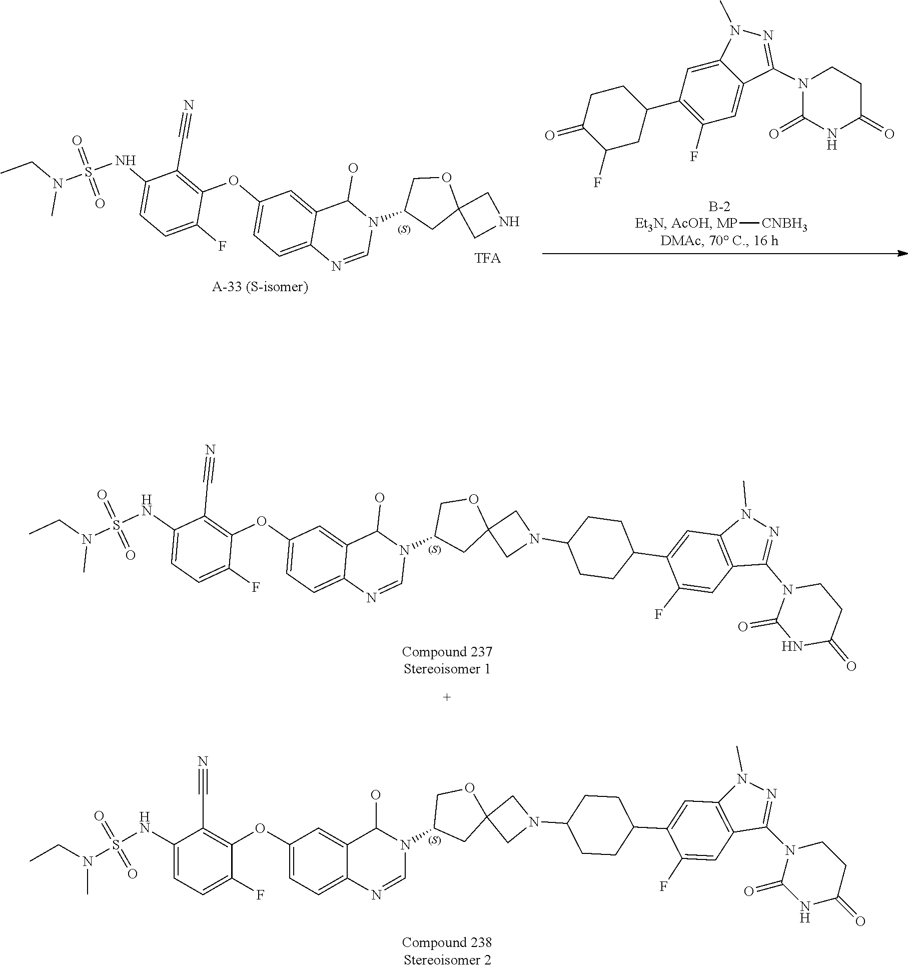

Into a 20 mL pressure vial containing a well-stirred solution of (7S)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-33) (600 mg, 933.74 mol, TFA salt) in DMAc (5 mL) was added triethylamine (933.74 mol, 130.14 μL) at room temperature and the resulting mixture was stirred for 5 min at the same temperature. After 5 min, AcOH (933.74 mol, 53.4 μL) followed by 1-[5-fluoro-1-methyl-6-(4-oxocyclohexyl)indazol-3-yl]hexahydropyrimidine-2,4-dione (B-2) (401.54 mg, 1.12 mmol) were added at 0° C. and stirred for 5 hours. Subsequently, MP-CNBH₃ (600 mg, 933.74 mol) was added and resulting reaction mixture was stirred at 70° C. for 16 hours. After completion of the reaction, the reaction mixture was filtered through a cotton pad, and the filtrate was concentrated under reduced pressure to get a crude residue which was purified by preparative HPLC [Column: X-BRIDGE C18 (250 mm*19 mm*5 μm), Mobile phase A: 10 mM NH₄HCO₃ in milli-Q water, Mobile phase B: 100% acetonitrile, flow rate: 15 mL/min]. The first eluting fraction (Peak-1) was lyophilized to afford Compound 237 (assigned as stereoisomer 1).

Peak-1: (7S)-7-[6-[2-cyano-3-[[ethyl(methyl)sulfamoyl]amino]-6-fluoro-phenoxy]-4-oxo-quinazolin-3-yl]-2-[4-[3-(2,4-dioxohexahydropyrimidin-1-yl)-5-fluoro-1-methyl-indazol-6-yl]cyclohexyl]-5-oxa-2-azaspiro[3.4]octane (Compound 237, 15 mg, 17.07 mol, 2% yield) as an off-white solid. UPLC-MS (ES+): m/z 871.2 [M+H]⁺. ¹H NMR (400 MHz, DMSO-d₆): δ 10.56 (s, 1H), 10.10 (bs, 1H), 8.21 (s, 1H), 7.78 (d, J=8.80 Hz, 1H), 7.69 (dd, J=3.20, 8.80 Hz, 1H), 7.53-7.40 (m, 2H), 7.39-7.25 (m, 3H), 5.20-5.10 (s, 1H), 4.55-3.88 (m, 12H), 3.05-2.95 (m, 3H), 2.90-2.77 (m, 4H), 2.61 (s, 3H), 2.10-1.90 (m, 4H), 1.68-1.55 (m, 2H), 1.38-1.22 (m, 2H), 1.03 (t, J=7.20 Hz, 3H).

Similarly, the second eluted fraction (Peak-2) was lyophilized to afford Compound 238 (assigned as stereoisomer 2).

Peak-2: (7S)-7-[6-[2-cyano-3-[[ethyl(methyl)sulfamoyl]amino]-6-fluoro-phenoxy]-4-oxo-quinazolin-3-yl]-2-[4-[3-(2,4-dioxohexahydropyrimidin-1-yl)-5-fluoro-1-methyl-indazol-6-yl]cyclohexyl]-5-oxa-2-azaspiro[3.4]octane (Compound 238.36 mg, 41.12 mol, 4% yield) as an off-white solid. UPLC-MS (ES+): m/z 871.2 [M+H]⁺. ¹H NMR (400 MHz, DMSO-d₆): δ 10.56 (s, 1H), 10.09 (bs, 1H), 8.21 (s, 1H), 7.78 (d, J=8.80 Hz, 1H), 7.70-7.33 (m, 6H), 5.25-5.15 (m, 1H), 4.55-3.88 (m, 10H), 3.60-3.45 (m, 1H), 3.10-2.85 (m, 3H), 2.76 (t, J=6.80 Hz, 2H), 2.69-2.62 (m, 4H), 1.92-1.53 (m, 8H), 1.03 (t, J=6.80 Hz, 3H).

### Synthesis D138: Synthesis of 6-[2-cyano-3-[[ethyl(methyl)sulfamoyl]amino]-6-fluoro-phenoxy]-3-[(1R,5S)-3-[4-[3-(2,4-dioxohexahydropyrimidin-1-yl)-5-fluoro-1-methyl-indazol-6-yl]cyclohexyl]-3-azabicyclo[3.1.0]hexan-6-yl]-4-oxo-quinazoline (Compound 239 Compound 240)

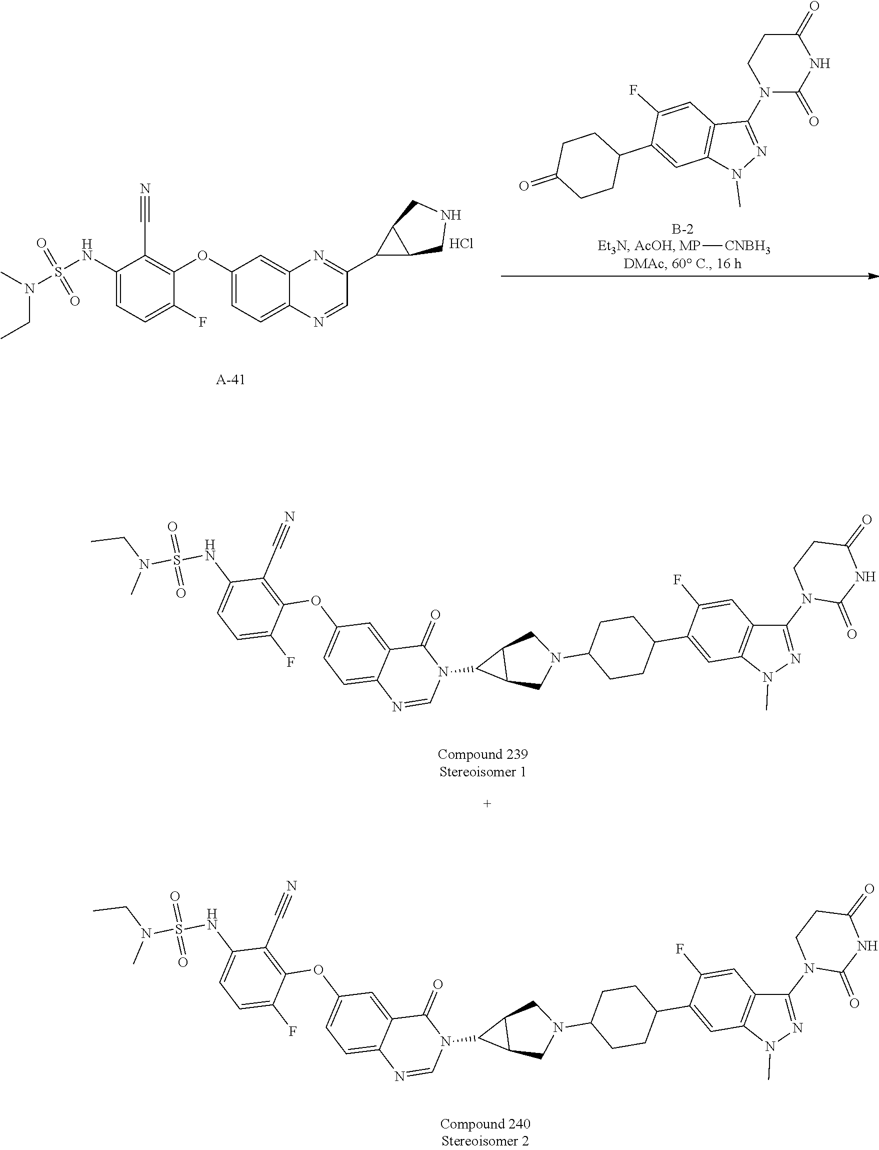

Into a 20 mL pressure vial containing a well-stirred solution of 3-[(1S,5R)-3-azabicyclo[3.1.0]hexan-6-yl]-6-[2-cyano-3-[[ethyl(methyl)sulfamoyl]amino]-6-fluoro-phenoxy]-4-oxo-quinazoline (A-41) (300 mg, 560.76 mol, HCl salt) in DMAc (4 mL) was added triethylamine (2.24 mmol, 312.63 μL) at room temperature and stirred for 20 min. Reaction mixture was cooled to 0° C., 1-[5-fluoro-1-methyl-6-(4-oxocyclohexyl)indazol-3-yl]hexahydropyrimidine-2,4-dione (B-2) (241.15 mg, 672.91 mol) and AcOH (2.24 mmol, 128.28 μL) were added and stirred for 3 h at room temperature. MP-CNBH₃ (300 mg, 5.61 mmol) was added and stirred at 60° C. for 16 hours. After completion of the reaction, the reaction mixture was filtered and the filtrate was concentrated under reduced pressure to get a crude residue which was purified by reverse phase column chromatography (Redisep R_{f}Gold®reversed-phase C18-teledyne ISCO, 120 g), [Mobile phase A: NH₄HCO₃ in milli-Q water; Mobile phase B: acetonitrile]. The fractions were lyophilized to afford 105 mg of mixture of cis and trans isomers. Further cis and trans isomers were separated by preparative HPLC [Column: X-Bridge C18 (250 mm×19 mm×5 m), Mobile phase A: 10 mM NH₄HCO₃ in milli-Q water; Mobile phase B: acetonitrile]. The first eluting fraction (Peak-1) was lyophilized to afford Compound 239 (assigned as stereoisomer 1).

Peak-1: 6-[2-cyano-3-[[ethyl(methyl)sulfamoyl]amino]-6-fluoro-phenoxy]-3-[(1R,5S)-3-[4-[3-(2,4-dioxohexahydropyrimidin-1-yl)-5-fluoro-1-methyl-indazol-6-yl]cyclohexyl]-3-azabicyclo[3.1.0]hexan-6-yl]-4-oxo-quinazoline (Compound 239, 15 mg, 17.64 mol, 3% yield) as an off-white solid. UPLC-MS (ES+): m/z 841.3 [M+H]⁺. ¹H NMR (400 MHz, DMSO-d₆): δ 10.55 (s, 1H), 9.97 (bs, 1H), 8.28 (s, 1H), 7.76 (d, J=8.80 Hz, 1H), 7.66-7.63 (m, 2H), 7.55 (d, J=6.00 Hz, 1H), 7.41-7.35 (m, 3H), 3.99 (s, 3H), 3.90 (t, J=6.40 Hz, 2H), 3.62-3.42 (m, 2H), 3.30-3.27 (m, 2H), 3.11-3.09 (m, 2H), 2.99-2.87 (m, 2H), 2.76-2.73 (m, 4H), 2.63-2.58 (m, 2H), 2.33-2.11 (m, 4H), 1.94-1.92 (m, 2H), 1.65-1.62 (m, 2H), 1.52-1.37 (m, 2H), 1.05 (t, J=7.20 Hz, 3H).

Similarly, the second eluted fraction (Peak-2) was lyophilized to afford Compound 240 (assigned as stereoisomer 2).

Peak-2: 6-[2-cyano-3-[[ethyl(methyl)sulfamoyl]amino]-6-fluoro-phenoxy]-3-[(1R,5S)-3-[4-[3-(2,4-dioxohexahydropyrimidin-1-yl)-5-fluoro-1-methyl-indazol-6-yl]cyclohexyl]-3-azabicyclo[3.1.0]hexan-6-yl]-4-oxo-quinazoline (Compound 240, 30 mg, 35.54 mol, 6% yield) as a white solid. UPLC-MS (ES+): m/z 841.3 [M+H]⁺. ¹H NMR (400 MHz, DMSO-d₆): δ 10.54 (s, 1H), 10.18 (bs, 1H), 8.33 (s, 1H), 7.78-7.76 (m, 1H), 7.66 (dd, J=2.80, 8.80 Hz, 1H), 7.43-7.41 (m, 2H), 7.36-7.34 (m, 2H), 3.99 (s, 3H), 3.89 (t, J=6.80 Hz, 2H), 3.44-3.38 (m, 1H), 3.29-3.27 (m, 3H), 3.22-3.09 (m, 2H), 3.14-3.12 (m, 2H), 2.93-2.87 (m, 1H), 2.79-2.67 (m, 5H), 2.43-2.38 (m, 2H), 2.33-2.08 (m, 2H), 1.98-1.88 (m, 4H), 1.61-1.48 (m, 3H), 1.05 (t, J=7.20 Hz, 3H).

### Synthesis D139: Synthesis of (1S,5R)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-azabicyclo[3.1.0]hexane-3-sulfonamide (Compound 241)

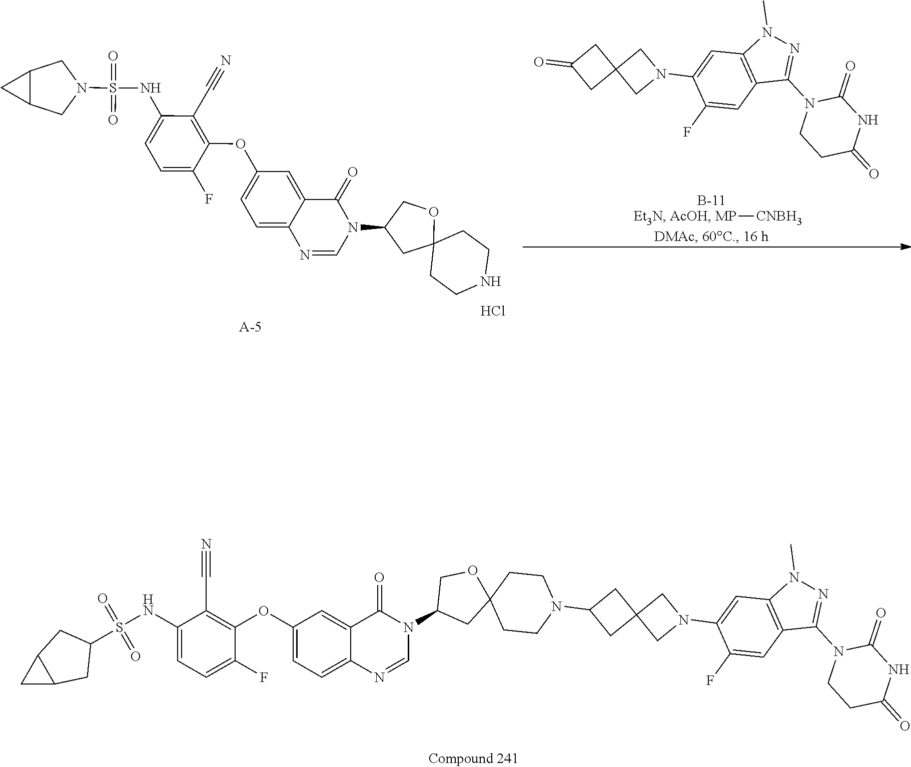

Into a 20 mL pressure vial containing a well stirred solution of N-[2-cyano-4-fluoro-3-[3-[(3R)-1-oxa-8-azaspiro[4.5]decan-3-yl]-4-oxo-quinazolin-6-yl]oxy-phenyl]-3-azabicyclo[3.1.0]hexane-3-sulfonamide (A-5) (282.00 mg, 456.98 mol, HCl salt) and 1-[5-fluoro-1-methyl-6-(6-oxo-2-azaspiro[3.3]heptan-2-yl)indazol-3-yl]hexahydropyrimidine-2,4-dione (B-11) (251.26 mg, 676.60 mol) in DMAc (5 mL) was added triethylamine (3.37 mmol, 470.10 μL) and resulting mixture was stirred at room temperature for 1 hour. After 1 h, AcOH (2.02 mmol, 115.73 μL) was added and stirred at room temperature for 2 hours. Subsequently, MP-CNBH₃ (0.300 g, 674.56 mol) was added and stirred at 60° C. for 16 hours. After completion the reaction, reaction mixture was filtered through sintered funnel and the filtrate was concentrated under reduced pressure to get crude which was purified by the reverse phase purification [Column: Redisep R_{f} Gold® C18-teledyne ISCO, 100 g, Mobile phase A: 10 mM NH₄HCO₃ in milli-Q water; Mobile phase B: acetonitrile]. Fractions were collected and lyophilized to afford (1S,5R)-N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-azabicyclo[3.1.0]hexane-3-sulfonamide (Compound 241, 0.074 g, 76.65 mol, 11% yield) as a white solid. UPLC-MS (ES+): m/z 936.3 [M+H]⁺. ¹H NMR (400 MHz, DMSO-d₆): δ 10.51 (s, 1H), 9.55 (bs, 1H), 8.31 (s, 1H), 7.76 (d, J=8.80 Hz, 1H), 7.64 (dd, J=2.80, 8.80 Hz, 1H), 7.55-7.45 (m, 1H), 7.35 (d, J=2.80 Hz, 1H), 7.30-7.25 (m, 2H), 6.52 (d, J=7.60 Hz, 1H), 5.33-5.25 (m, 1H), 4.20-4.08 (m, 2H), 4.02 (s, 2H), 3.95-3.85 (m, 7H), 3.17 (m, 4H), 3.00-2.70 (m, 5H), 2.43-2.35 (m, 2H), 2.30-2.21 (m, 2H), 2.16-2.07 (m, 1H), 1.97-1.65 (m, 4H), 1.50-1.44 (m, 2H), 0.51-0.45 (m, 1H), 0.37-0.33 (m, 1H).

### Synthesis D140: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]morpholine-4-sulfonamide (Compound 242)

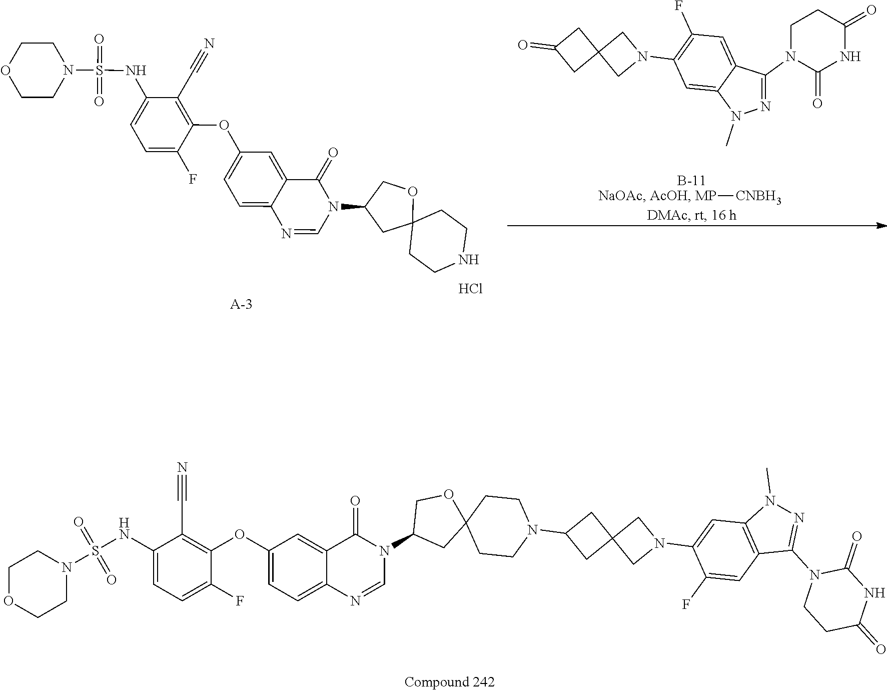

Into a 20 mL pressure vial containing a well-stirred solution of N-[2-cyano-4-fluoro-3-[3-[(3R)-1-oxa-8-azaspiro[4.5]decan-3-yl]-4-oxo-quinazolin-6-yl]oxy-phenyl]tetrahydropyran-4-sulfonamide (A-3) (150 mg, 214.49 mol, HCl salt) and 1-[5-fluoro-1-methyl-6-(6-oxo-2-azaspiro[3.3]heptan-2-yl)indazol-3-yl]hexahydropyrimidine-2,4-dione (B-11) (95.59 mg, 257.39 mol) in DMAc (4 mL) were added AcOH (643.48 mol, 36.80 μL) and anhydrous NaOAc (35.19 mg, 428.99 mol). Resulting mixture was stirred at room temperature for 2 hours. Subsequently, MP-CNBH₃ (250 mg, 214.49 mol) was added and stirred at room temperature for 16 hours. After completion of the reaction, the reaction mixture was filtered through Celite bed and washed with ethyl acetate (20 mL). Filtrate was concentrated under reduced pressure to get crude residue which was purified by preparative HPLC [Column: X Bridge C18 (150 mm*19 mm*5 μm), Mobile phase A: 10 mM NH₄HCO₃ (pH was adjusted to 10 with NH₄OH) in milli-Q water; Mobile phase B: acetonitrile, flow rate:15 mL/min]. Fractions were collected and lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]morpholine-4-sulfonamide (Compound 242, 32 mg, 33.49 mol, 16% yield) as an off-white solid. UPLC-MS (ES+): m/z 940.3 [M+H]⁺. ¹H NMR (400 MHz, DMSO-d₆): δ 10.51 (s, 1H), 9.28 (bs, 1H), 8.31 (s, 1H), 7.76 (d, J=8.80 Hz, 1H), 7.64 (dd, J=3.20, 9.00 Hz, 1H), 7.39-7.26 (m, 4H), 6.53 (dd, J=7.60 Hz, 1H), 5.30-5.27 (m, 1H), 4.31-4.08 (m, 2H), 4.03 (s, 2H), 3.98-3.86 (m, 7H), 3.62-3.55 (m, 5H), 3.00-2.91 (m, 5H), 2.88-2.71 (m, 4H), 2.65-2.57 (m, 4H), 2.44-2.34 (m, 2H), 2.27-1.61 (m, 5H).

### Synthesis D141: Synthesis of 6-[2-cyano-3-[[ethyl(methyl)sulfamoyl]amino]-6-fluoro-phenoxy]-4-[[7-[4-[3-(2,4-dioxohexahydropyrimidin-1-yl)-5-fluoro-1-methyl-indazol-6-yl]cyclohexyl]-7-azaspiro[3.5]nonan-2-yl]oxy]quinazoline (Compound 243 and Compound 244)

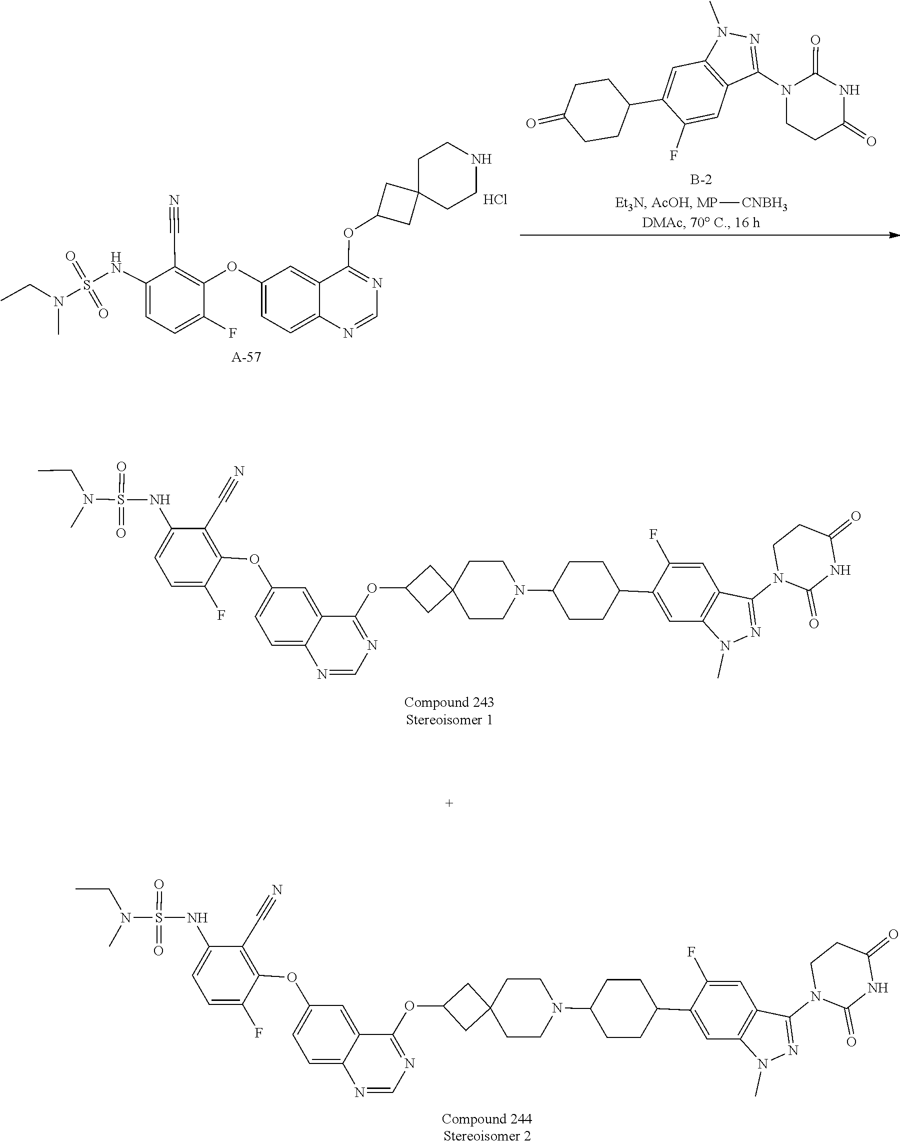

Into a 40 mL glass vial with pressure released cap containing a well-stirred solution of 4-(7-azaspiro[3.5]nonan-2-yloxy)-6-[2-cyano-3-[[ethyl(methyl)sulfamoyl]amino]-6-fluoro-phenoxy]quinazoline (A-57) (290 mg, 391.98 mol, HCl salt) in DMAc (6 mL) was added triethylamine (1.96 mmol, 273.17 μL) at room temperature and stirred for 10 min. Subsequently, AcOH (1.18 mmol, 67.25 μL) and 1-[5-fluoro-1-methyl-6-(4-oxocyclohexyl)indazol-3-yl]hexahydropyrimidine-2,4-dione (B-2) (210.71 mg, 587.97 mol) were added and stirred at room temperature for 2 hours. MP-CNBH₃ (391.98 mol) was added, and the resulting reaction mixture was stirred at 70° C. for 16 hours. After completion of the reaction, the reaction mixture was filtered and filtrate was concentrated under reduced pressure to get a crude residue which was purified by preparative HPLC [Column: X-Bridge C18 (150 mm*19 mm*5 μm), Mobile phase A: 10 mM NH₄HCO₃ & NH₄OH (pH 10) in milli-Q water; Mobile phase B: acetonitrile]. The first eluting fraction (Peak-1) was lyophilized to afford Compound 243 (assigned as stereoisomer 1).

Peak-1: 6-[2-cyano-3-[[ethyl(methyl)sulfamoyl]amino]-6-fluoro-phenoxy]-4-[[7-[4-[3-(2,4-dioxohexahydropyrimidin-1-yl)-5-fluoro-1-methyl-indazol-6-yl]cyclohexyl]-7-azaspiro[3.5]nonan-2-yl]oxy]quinazoline (Compound 243, 36 mg, 39.80 mol, 10% yield) as an off-white solid. UPLC-MS (ES+): m/z 883.3 [M+H]⁺. ¹H NMR (400 MHz, DMSO-d₆): δ 10.56 (s, 1H), 8.98 (bs, 1H), 8.73 (s, 1H), 7.99 (d, J=9.20 Hz, 1H), 7.77 (dd, J=2.80, 9.20 Hz, 1H), 7.56 (d, J=6.00 Hz, 1H), 7.40-7.26 (m, 4H), 5.43-5.40 (m, 1H), 4.00 (s, 3H), 3.91 (t, J=6.80 Hz, 2H), 3.24-2.85 (m, 7H), 2.76 (t, J=6.80 Hz, 2H), 2.58-2.56 (m, 4H), 2.24-1.75 (m, 10H), 1.71-1.61 (m, 4H), 1.03 (t, J=7.20 Hz, 3H).

Similarly, the second eluted fraction (Peak-2) was lyophilized to afford Compound 244 (assigned as stereoisomer 2).

Peak-2: 6-[2-cyano-3-[[ethyl(methyl)sulfamoyl]amino]-6-fluoro-phenoxy]-4-[[7-[4-[3-(2,4-dioxohexahydropyrimidin-1-yl)-5-fluoro-1-methyl-indazol-6-yl]cyclohexyl]-7-azaspiro[3.5]nonan-2-yl]oxy]quinazoline (Compound 244, 37 mg, 41.76 mol, 11% yield) as an off-white solid. UPLC-MS (ES+): m/z 883.3 [M+H]⁺. ¹H NMR (400 MHz, DMSO-d₆): δ 10.56 (s, 1H), 8.72 (s, 1H), 8.53 (bs, 1H), 7.99 (d, J=9.20 Hz, 1H), 7.76 (dd, J=2.80, 9.20 Hz, 1H), 7.49-7.29 (m, 5H), 5.44-5.37 (m, 1H), 4.03 (s, 3H), 3.91 (t, J=6.40 Hz, 2H), 3.59-3.43 (m, 3H), 3.23-2.85 (m, 5H), 2.76 (t, J=6.80 Hz, 2H), 2.59-2.54 (m, 4H), 2.44-2.41 (m, 1H), 2.14-1.75 (m, 14H), 1.03 (t, J=7.20 Hz, 3H).

### Synthesis D142: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-5-azaspiro[2.4]heptane-5-sulfonamide (Compound 245)

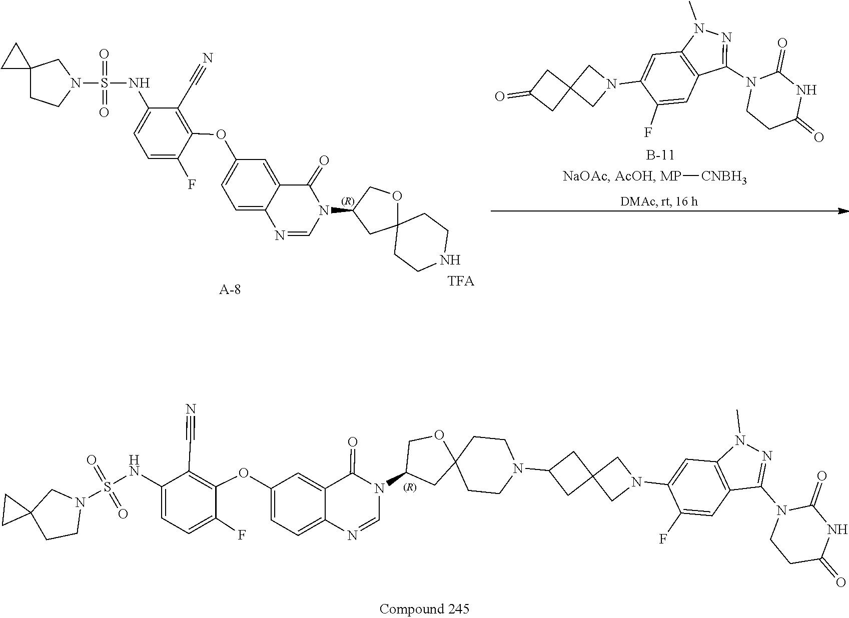

Into a 40 mL glass vial containing a well stirred solution N-[2-cyano-4-fluoro-3-[3-[(3R)-1-oxa-8-azaspiro[4.5]decan-3-yl]-4-oxo-quinazolin-6-yl]oxy-phenyl]-5-azaspiro[2.4]heptane-5-sulfonamide (A-8) (180.00 mg, 251.45 mol, TFA salt) and 1-[5-fluoro-1-methyl-6-(6-oxo-2-azaspiro[3.3]heptan-2-yl)indazol-3-yl]hexahydropyrimidine-2,4-dione (B-11) (102.72 mg, 276.60 mol) in anhydrous DMAc (4 mL) were added NaOAc (103.13 mg, 1.26 mmol) followed by AcOH (754.36 mol, 43.14 μL) at room temperature and stirred for 10 min. Finally, MP-CNBH₃ (180 mg, 251.45 mol) was added and stirred at room temperature for 16 hours. After completion of the reaction, the reaction mass was diluted with dichloromethane (10 mL) and filtered, filtrate was concentrated under reduced pressure to afford crude which was purified by prep HPLC [Column: X-Bridge C8 (250 mm*19 mm*5 μm), Mobile phase A: 10 mM NH₄HCO₃ and NH₄OH (pH 10) in milli-Q water; Mobile phase B: 100% acetonitrile]. The fraction was lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-5-azaspiro[2.4]heptane-5-sulfonamide (Compound 245, 75 mg, 78.06 mol, 31% yield) as an off-white solid. UPLC-MS (ES+): m/z 950.2 [M+H]⁺. ¹H NMR (400 MHz, DMSO-d₆): δ 10.51 (s, 1H), 9.68 (bs, 1H), 8.32 (s, 1H), 7.78-7.76 (m, 1H), 7.66 (dd, J=2.80, 9.00 Hz, 1H), 7.53-7.28 (m, 3H), 7.27 (d, J=12.8 Hz, 1H), 6.52 (d, J=7.60 Hz, 1H), 5.32-5.25 (m, 1H), 4.15-4.10 (m, 2H), 4.02 (s, 2H), 3.92-3.86 (m, 7H), 3.29-3.27 (m, 2H), 3.15-2.82 (m, 4H), 2.73 (t, J=6.80 Hz, 3H), 2.65-2.53 (m, 2H), 2.45-2.32 (m, 3H), 2.28-2.22 (m, 2H), 2.12-2.05 (m, 1H), 1.94-1.68 (m, 6H), 0.56-0.52 (m, 4H).

### Synthesis D143: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-methoxy-azetidine-1-sulfonamide (Compound 246)

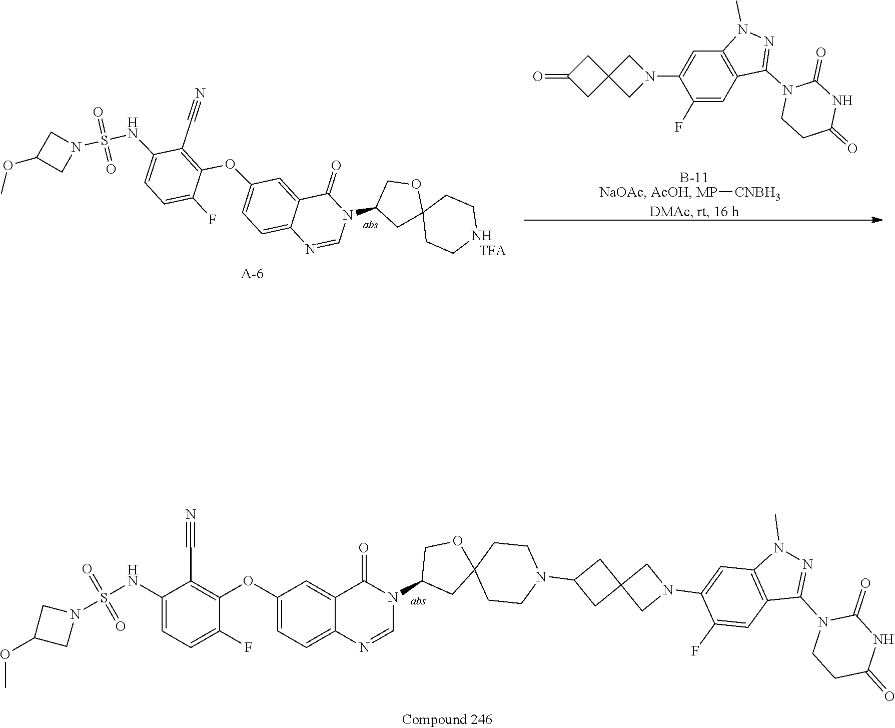

Into a 10 mL pressure vial containing a well stirred solution of N-[2-cyano-4-fluoro-3-[3-[(3R)-1-oxa-8-azaspiro[4.5]decan-3-yl]-4-oxo-quinazolin-6-yl]oxy-phenyl]-3-methoxy-azetidine-1-sulfonamide (A-6) (130 mg, 165.61 mol, TFA salt) and 1-[5-fluoro-1-methyl-6-(6-oxo-2-azaspiro[3.3]heptan-2-yl)indazol-3-yl]hexahydropyrimidine-2,4-dione (B-11) (92.25 mg, 248.41 mol) in anhydrous DMAc (2 mL) was added NaOAc (67.93 mg, 828.04 mol) followed by AcOH (496.82 mol, 41.10 μL) and stirred at room temperature for 30 min. Finally, MP-CNBH₃ (200 mg, 165.61 mol) was added and stirred at room temperature for 16 hours. After completion of the reaction, the reaction mixture was diluted with dichloromethane (10 mL) and filtered, filtrate was concentrated under reduced pressure to afford crude which was purified by preparative HPLC by using a method [Column: X-Bridge C8 (250 mm*19 mm*5 μm), Mobile phase A: 10 mM NH₄HCO₃ & NH₄OH (pH 10) in milli-Q water; Mobile phase B: acetonitrile]. The fraction was lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-3-methoxy-azetidine-1-sulfonamide (Compound 246, 62 mg, 65.79 mol, 40% yield) as an off-white solid. UPLC-MS (ES+): m/z 940.2 [M+H]⁺. ¹H NMR (400 MHz, DMSO-d₆): δ 10.51 (s, 1H), 9.34 (bs, 1H), 8.31 (s, 1H), 7.76 (d, J=9.20 Hz, 1H), 7.64 (dd, J=3.20, 8.80 Hz, 1H), 7.45-7.24 (m, 4H), 6.53 (d, J=4.00 Hz, 1H), 5.38-5.12 (m, 1H), 4.19-4.00 (m, 5H), 3.98-3.84 (m, 7H), 3.75-3.65 (m, 2H), 3.55-3.45 (m, 2H), 3.16 (s, 3H), 2.98-2.82 (m, 2H), 2.73 (t, J=6.40 Hz, 2H), 2.60-2.57 (m, 1H), 2.43-2.30 (m, 3H), 2.19-1.60 (m, 6H).

### Synthesis D144: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]propane-2-sulfonamide (Compound 247)

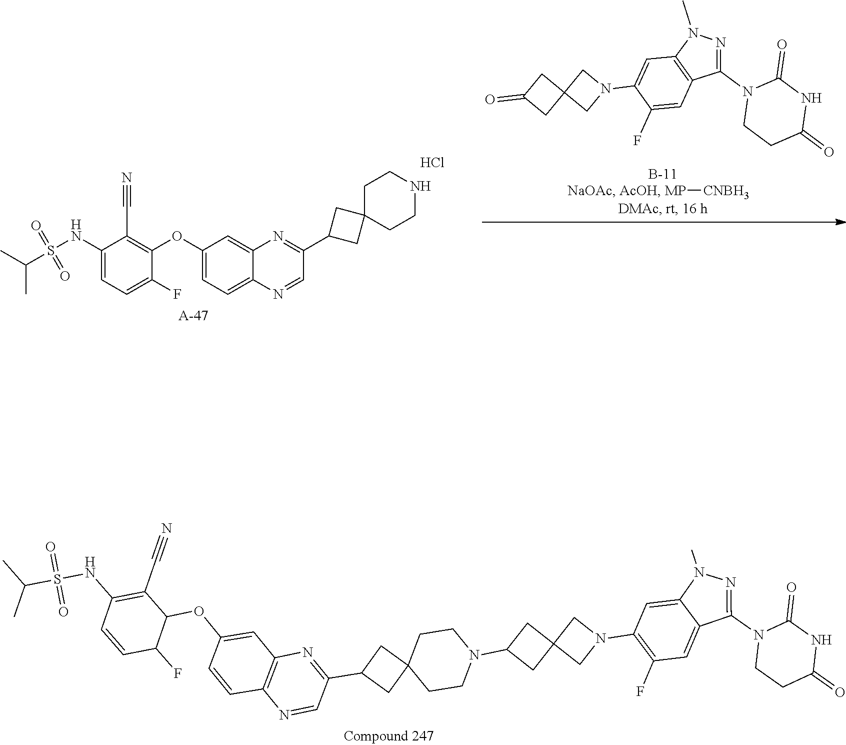

Into a 20 mL glass vial with pressure released cap containing a well-stirred solution of N-[3-[3-(7-azaspiro[3.5]nonan-2-yl)quinoxalin-6-yl]oxy-2-cyano-4-fluoro-phenyl]propane-2-sulfonamide (A-47) (150 mg, 258.22 mol, HCl salt) in DMAc (3 mL) was added NaOAc (63.54 mg, 774.61 mol) at room temperature and stirred for 5 min. Subsequently, AcOH (1.29 mmol, 73.83 μL) and 1-[5-fluoro-1-methyl-6-(6-oxo-2-azaspiro[3.3]heptan-2-yl)indazol-3-yl]hexahydropyrimidine-2,4-dione (B-11) (150.69 mg, 284.04 mol) were added at room temperature and stirred for 2 hours. Finally, MP-CNBH₃ (150 mg, 258.22 mol) was added and stirred at room temperature for 16 hours. After completion of the reaction, the reaction mixture was filtered on cotton pad and filtrate was concentrated under reduced pressure to get crude residue, which was purified by reverse phase column using method (Column: Redisep R_{f} Gold®reversed-phase C18-teledyne ISCO, 120 g), [Mobile phase A: 10 mM NH₄HCO₃ in milli-Q water; Mobile phase B: acetonitrile]. Collected fractions were dried to afford 90 mg of desired product, which is not pure. Further purified by prep HPLC by using a method [Column: X-Bridge C18 (150 mm×19 mm×5 μm) and Mobile phase A: 10 mM NH₄HCO₃ (pH-10 adjusted with NH₄OH) in milli-Q water; Mobile phase B: acetonitrile]. Collected fractions were lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]propane-2-sulfonamide (Compound 247, 37 mg, 42.52 mol, 16% yield) as an off-white solid. UPLC-MS (ES+): m/z 865.4 [M+H]⁺. ¹H NMR (400 MHz, DMSO-d₆): δ 10.51 (s, 1H), 9.34 (bs, 1H), 8.78 (s, 1H), 8.12 (d, J=9.20 Hz, 1H), 7.68 (dd, J=2.80, 9.20 Hz, 1H), 7.43-7.36 (m, 2H), 7.29-7.26 (m, 1H), 7.13 (bs, 1H), 6.52 (d, J=7.20 Hz, 1H), 4.03 (s, 2H), 3.93-3.86 (m, 8H), 3.60-3.40 (m, 2H), 3.10-2.90 (m, 3H), 2.73 (t, J=6.80 Hz, 2H), 2.68-2.68 (m, 1H), 2.50-2.40 (m, 2H), 2.34-2.23 (m, 6H), 1.92-1.69 (m, 4H), 1.24 (d, J=6.80 Hz, 6H).

### Synthesis D145: Synthesis of N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]propane-2-sulfonamide (Compound 248 and Compound 249)

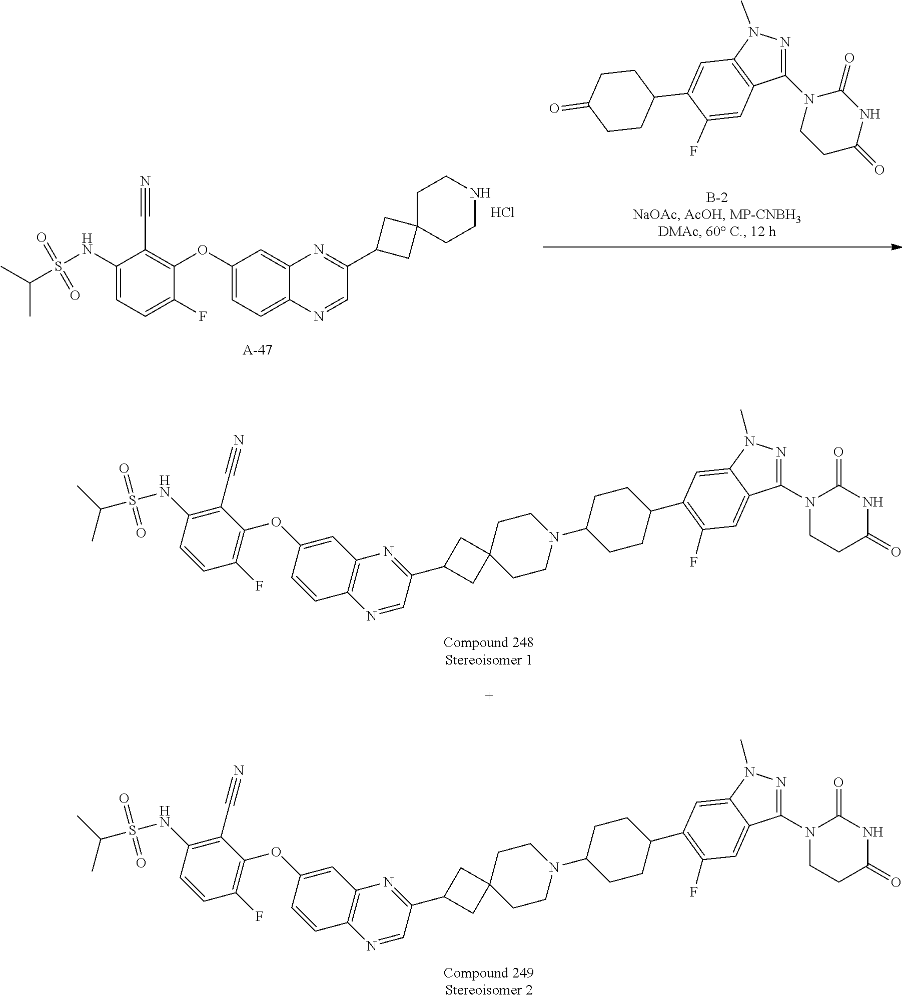

Into a 20 mL glass vial with pressure released cap containing a well-stirred solution of N-[3-[3-(7-azaspiro[3.5]nonan-2-yl)quinoxalin-6-yl]oxy-2-cyano-4-fluoro-phenyl]propane-2-sulfonamide (A-47) (400 mg, 688.57 mol, HCl salt) in DMAc (5 mL) was added NaOAc (169.45 mg, 2.07 mmol) at room temperature and stirred for 10 min. Subsequently, AcOH (3.44 mmol, 196.89 μL) and 1-[5-fluoro-1-methyl-6-(4-oxocyclohexyl)indazol-3-yl]hexahydropyrimidine-2,4-dione (B-2) (296.11 mg, 826.29 mol) were added at room temperature and stirred for 2 hours. Finally, MP-CNBH₃ (400 mg, 688.57 mol) was added at room temperature and stirred at 60° C. for 12 hours. After completion of the reaction, the reaction mixture was filtered through cotton pad and filtrate was concentrated under reduced pressure to get a crude residue, which was purified by prep HPLC by using a method Column: X Bridge C18 (150 mm×19 mm×5 μm) and [Mobile phase A:10 mM NH₄HCO₃ (pH-10 adjusted with NH₄OH) in milli-Q water; Mobile phase B: acetonitrile]. First eluted fraction (Peak-1) was lyophilized to afford Compound 248 (assigned as stereoisomer 1).

Peak-1: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]propane-2-sulfonamide (Compound 248, 70 mg, 81.74 mol, 12% yield) as an off-white solid. UPLC-MS (ES+): m/z 852.3 [M+H]⁺. ¹H NMR (400 MHz, DMSO-d₆): δ 10.56 (s, 1H), 8.97 (bs, 1H), 8.77 (s, 1H), 8.12 (d, J=9.20 Hz, 1H), 7.68 (dd, J=2.80, 9.20 Hz, 1H), 7.54 (d, J=5.60 Hz, 1H), 7.40-7.32 (m, 3H), 7.10 (bs, 1H), 4.00 (s, 3H), 3.96-3.89 (m, 4H), 3.60-3.40 (m, 1H), 3.33-2.92 (m, 6H), 2.76 (t, J=6.40 Hz, 2H), 2.40-2.10 (m, 6H), 2.10-1.90 (m, 2H), 1.71-1.68 (m, 7H), 1.22 (d, J=6.40 Hz, 6H).

Similarly, the second eluted fraction (Peak-2) was lyophilized to afford Compound 249 (assigned as stereoisomer 2).

Peak-2: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]propane-2-sulfonamide (Compound 249, 50 mg, 58.62 mol, 9% yield) as an off-white solid. UPLC-MS (ES+): m/z 852.3 [M+H]⁺. ¹H NMR (400 MHz, DMSO-d₆): δ 10.56 (s, 1H), 8.77 (s, 1H), 8.54 (bs, 1H), 8.12 (d, J=9.20 Hz, 1H), 7.68 (dd, J=2.80, 9.20 Hz, 1H), 7.50-7.30 (m, 4H), 7.11 (bs, 1H), 4.03 (s, 3H), 3.95-3.90 (m, 3H), 3.60-3.50 (m, 1H), 3.18-2.74 (m, 4H), 2.68 (t, J=1.60 Hz, 2H), 2.34-1.87 (m, 18H), 1.23 (d, J=6.40 Hz, 6H).

### Synthesis D146: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]cyclopentanesulfonamide (Compound 250)

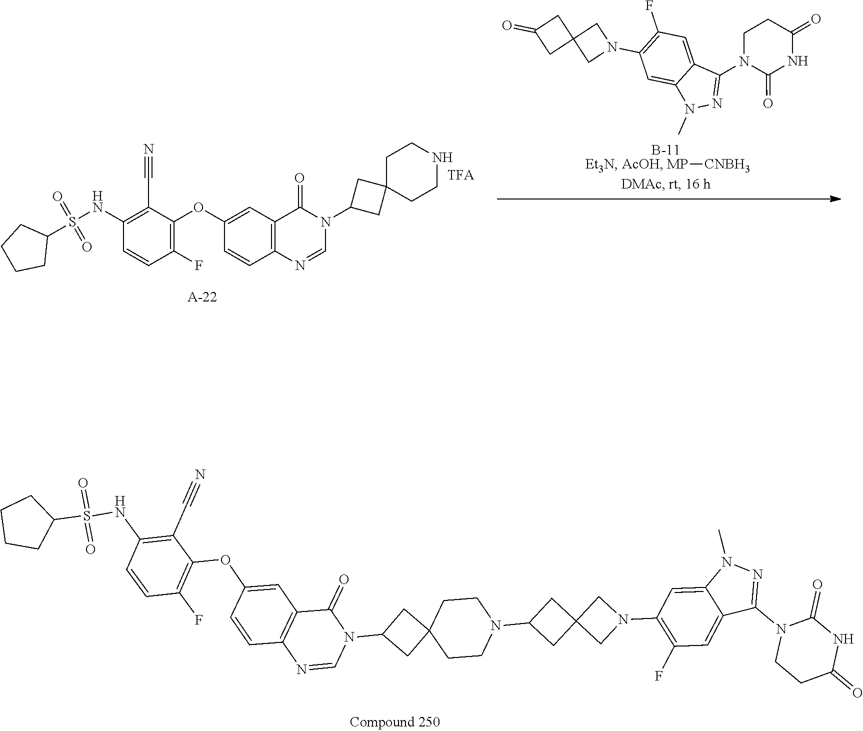

Into a 20 mL pressure capped vial containing a well-stirred solution of N-[3-[3-(7-azaspiro[3.5]nonan-2-yl)-4-oxo-quinazolin-6-yl]oxy-2-cyano-4-fluoro-phenyl]cyclopentanesulfonamide (A-22) (120 mg, 169.46 mol, TFA salt) in anhydrous DMAc (3 mL) was added triethylamine (847.29 mol, 118.09 μL) and stirred for 10 min. After 10 min, 1-[5-fluoro-1-methyl-6-(6-oxo-2-azaspiro[3.3]heptan-2-yl)indazol-3-yl]hexahydropyrimidine-2,4-dione (B-11) (74.04 mg, 169.46 mol) and AcOH (508.37 mol, 30.53 μL) were added and stirred for 30 min. Subsequently, MP-CNBH₃ (120 mg) was added, and the resulting reaction mixture was stirred at room temperature for 16 hours. After completion of the reaction, the reaction mixture was filtered through sintered funnel and filtrate was concentrated under reduced pressure to get crude which was purified by reverse phase purification [Column: Redisep R_{f} Gold® reversed-phase C18-teledyne ISCO, 120 g), Mobile phase A: 10 mM NH₄HCO₃ in milli-Q water; Mobile phase B: acetonitrile]. Fractions were collected and lyophilized to afford product, which is impure. Further re-purified by preparative HPLC [Column: X BRIDGE C18 (150 mm*21 mm*5 μm), Mobile phase A: 10 mM NH₄HCO₃ in milli-Q water; Mobile phase B: acetonitrile]. Fractions were collected and lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]cyclopentanesulfonamide (Compound 250, 41 mg, 45.00 mol, 27% yield) as an off-white solid. UPLC-MS (ES+): m/z 907.3 [M+H]⁺. ¹H NMR (400 MHz, DMSO-d₆): δ 10.51 (s, 1H), 9.48 (bs, 1H), 8.39 (s, 1H), 7.76 (d, J=8.80 Hz, 1H), 7.63 (dd, J=2.80, 8.80 Hz, 1H), 7.60-7.25 (m, 4H), 6.52 (d, J=7.60 Hz, 1H), 5.03-4.58 (m, 1H), 4.02 (s, 2H), 3.95-3.85 (m, 7H), 3.48-3.35 (m, 4H), 2.73 (t, J=6.80 Hz, 2H), 2.45-2.25 (m, 8H), 1.98-1.63 (m, 10H), 1.58-1.48 (m, 2H).

### Synthesis D147: Synthesis of (3R)-3-[6-[2-cyano-3-[[ethyl(methyl)sulfamoyl]amino]-6-fluoro-phenoxy]-4-oxo-quinazolin-3-yl]-8-[[1-[3-(2,4-dioxohexahydropyrimidin-1-yl)-5-fluoro-1-methyl-indazol-6-yl]-4-piperidyl]methyl]-1-oxa-8-azaspiro[4.5]decane (Compound 251)

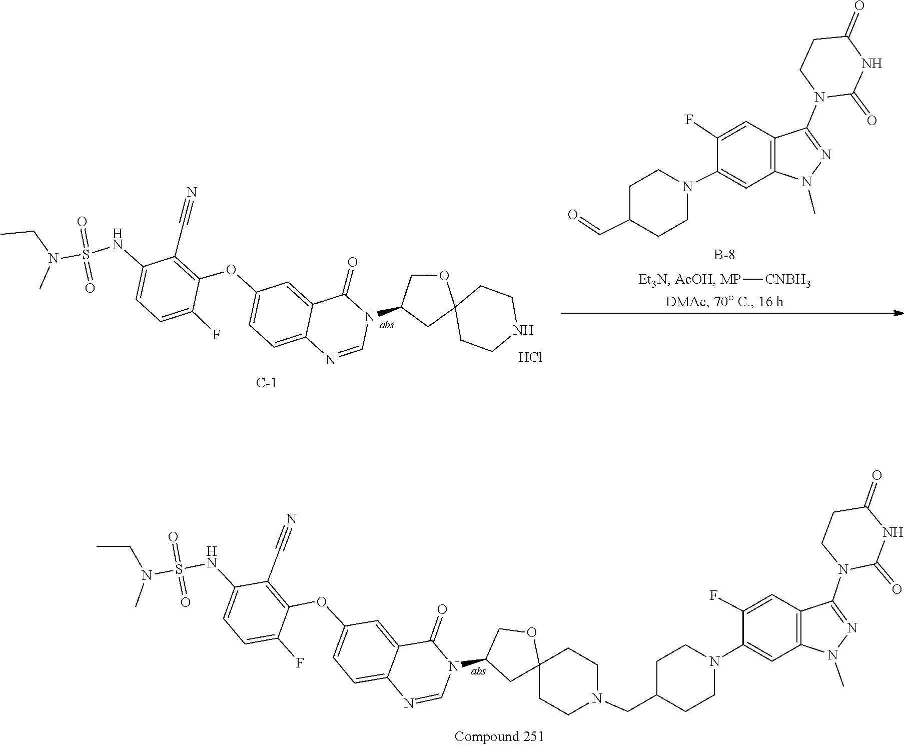

Into a 8 mL glass vial containing a well-stirred solution of 1-[3-(2,4-dioxohexahydropyrimidin-1-yl)-5-fluoro-1-methyl-indazol-6-yl]piperidine-4-carbaldehyde (B-8) (75 mg, 200.87 mol) and (3R)-3-[6-[2-cyano-3-[[ethyl(methyl)sulfamoyl]amino]-6-fluoro-phenoxy]-4-oxo-quinazolin-3-yl]-1-oxa-8-azaspiro[4.5]decane (C-1) (119.13 mg, 200.87 mol, HCl salt) in anhydrous DMAc (3 mL) were added triethylamine (803.47 mol, 111.99 μL) and AcOH (401.73 mol, 22.97 μL) and stirred for 30 min. Subsequently, MP-CNBH₃ (75 mg, 200.87 mol) was added and stirred at 70° C. for 16 hours. After completion of the reaction, the reaction mixture was filtered, and filtrate was evaporated under reduced pressure to get a crude residue which was purified by reverse phase by using RediSep R_{f} Gold® 100 g column and [Mobile phase A: 10 mM NH₄HCO₃ in milli-Q water; Mobile phase B: acetonitrile]. Eluted fractions were lyophilized to afford (3R)-3-[6-[2-cyano-3-[[ethyl(methyl)sulfamoyl]amino]-6-fluoro-phenoxy]-4-oxo-quinazolin-3-yl]-8-[[1-[3-(2,4-dioxohexahydropyrimidin-1-yl)-5-fluoro-1-methyl-indazol-6-yl]-4-piperidyl]methyl]-1-oxa-8-azaspiro[4.5]decane (Compound 251, 33 mg, 35.94 mol, 18% yield) as an off-white solid. UPLC-MS (ES+): m/z 914.2 [M+H]⁺. ¹H NMR (400 MHz, DMSO-d₆): δ 10.53 (s, 1H), 8.33 (s, 1H), 7.77 (d, J=8.80 Hz, 1H), 7.65 (dd, J=2.80, 9.00 Hz, 1H), 7.50 (bs, 1H), 7.36-7.33 (m, 3H), 7.09 (d, J=7.20 Hz, 1H), 5.32-5.26 (m, 1H), 4.17-4.09 (m, 2H), 3.95 (s, 3H), 3.90 (t, J=6.80 Hz, 2H), 3.47-3.33 (m, 2H), 2.76-2.63 (m, 14H), 2.42-2.33 (m, 1H), 1.92-1.76 (m, 9H), 1.41 (m, 2H), 1.04 (t, J=7.20 Hz, 3H).

### Synthesis D148: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]tetrahydropyran-4-sulfonamide (Compound 252)

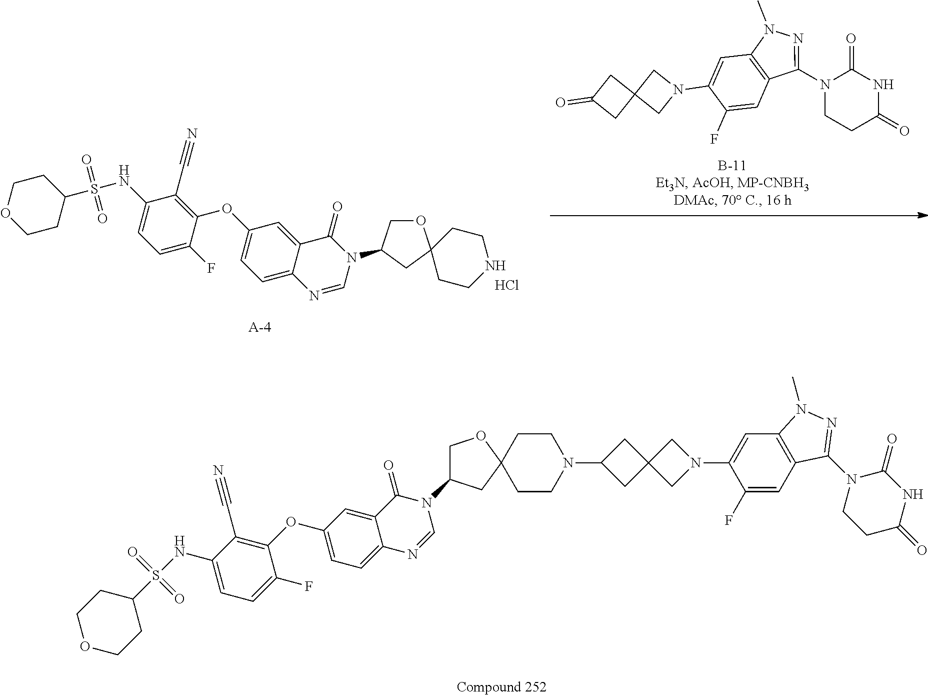

Into a 20 mL pressure vial containing a well-stirred solution of N-[2-cyano-4-fluoro-3-[3-[(3R)-1-oxa-8-azaspiro[4.5]decan-3-yl]-4-oxo-quinazolin-6-yl]oxy-phenyl]tetrahydropyran-4-sulfonamide (A-4) (150.00 mg, 241.90 mol, HCl salt) in DMAc (3.01 mL) was added triethylamine (725.70 mol, 101.15 μL) at room temperature under nitrogen atmosphere and mixture was stirred for 30 min. Reaction mixture was cooled to 0° C. and was added 1-[5-fluoro-1-methyl-6-(6-oxo-2-azaspiro[3.3]heptan-2-yl)indazol-3-yl]hexahydropyrimidine-2,4-dione 2 (B-11) (89.83 mg, 241.90 mol) followed by AcOH (1.21 mmol, 69.17 μL) and resulting mixture was stirred for 2 h at room temperature. Finally, MP-CNBH₃ (150 mg, 241.90 mol) was added, and the reaction mixture was stirred at 70° C. for 16 hours. After completion of the reaction, the reaction mixture was filtered and filtrate was concentrated under reduced pressure to get a crude residue which was purified by reverse phase column (Redisep R_{f} Gold®reversed-phase C18-teledyne ISCO, 120 g), [Mobile phase A: 10 mM NH₄HCO₃ in milli-Q water; Mobile phase B: acetonitrile]. The fractions were lyophilized to afford impure gummy compound which was again purified by preparative HPLC [Column: X-Bridge C18 (150 mm*19 mm*5 m), Mobile phase A: 10 mM NH₄HCO₃ in milli-Q water (pH 10 adjusted with ammonia); Mobile phase B: acetonitrile]. The eluting fraction was lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]tetrahydropyran-4-sulfonamide (Compound 252, 58 mg, 61.30 mol, 25% yield) as an off-white solid. UPLC-MS (ES+): m/z 939.2 [M+H]⁺. ¹H NMR (400 MHz, DMSO-d₆): δ 10.50 (s, 1H), 9.37 (s, 1H), 8.31 (s, 1H), 7.76 (d, J=9.20 Hz, 1H), 7.64 (dd, J=2.80, 8.80 Hz, 1H), 7.32 (s, 2H), 7.27 (d, J=12.80 Hz, 1H), 6.52 (d, J=7.60 Hz, 1H), 5.29 (bs, 1H), 3.90-4.27 (m, 4H), 3.82-3.98 (m, 9H), 3.22-3.33 (m, 5H), 3.12-3.81 (m, 3H), 2.71-2.76 (m, 2H), 2.36-2.48 (m, 3H), 1.78-2.32 (m, 8H), 1.57-1.77 (m, 3H).

### Synthesis D149: Synthesis of 6-[2-cyano-3-[[ethyl(methyl)sulfamoyl]amino]-6-fluoro-phenoxy]-3-[7-[1-[3-(2,4-dioxohexahydropyrimidin-1-yl)-5-fluoro-1-methyl-indazol-6-yl]-3,3-difluoro-4-piperidyl]-7-azaspiro[3.5]nonan-2-yl]-4-oxo-quinazoline (Compound 253)

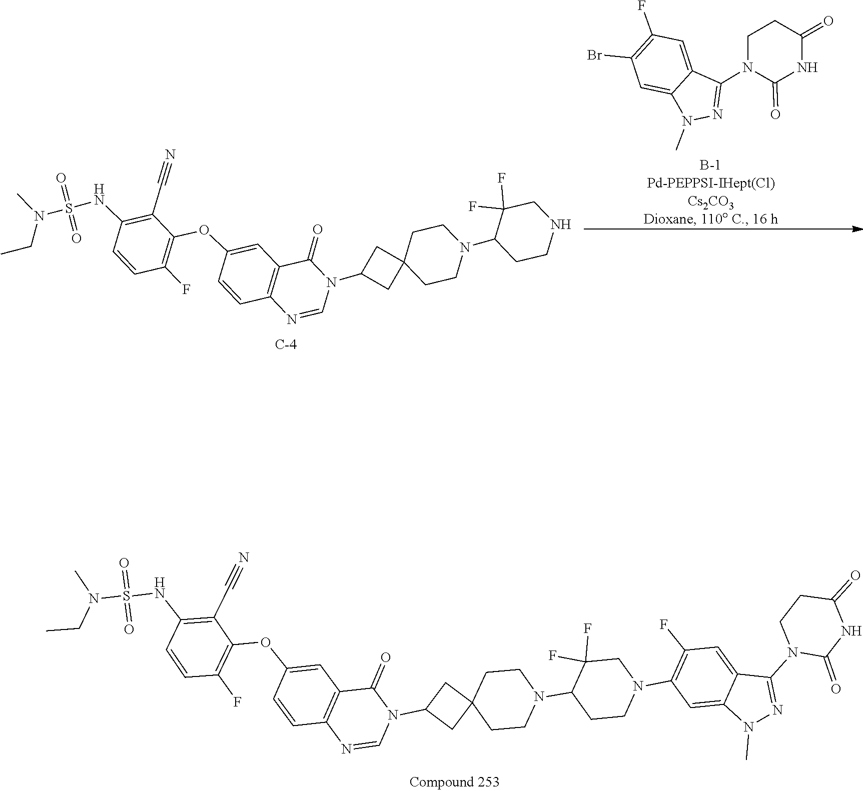

Into a 100 mL Sealed tube containing a well-stirred solution of 1-(6-bromo-5-fluoro-1-methyl-indazol-3-yl)hexahydropyrimidine-2,4-dione (B-1) (96.95 mg, 284.21 mol) in anhydrous 1,4-dioxane (30 mL) was added Cs₂CO₃ (111.12 mg, 341.05 mol) at room temperature under nitrogen atmosphere and resulting mixture was degassed by bubbling nitrogen gas into the reaction mixture for 5 min. Subsequently, 6-[2-cyano-3-[[ethyl(methyl)sulfamoyl]amino]-6-fluoro-phenoxy]-3-[7-(3,3-difluoro-4-piperidyl)-7-azaspiro[3.5]nonan-2-yl]-4-oxo-quinazoline (C-4, 100 mg, 113.68 mol,) was added to the reaction mixture and reaction mixture was again degassed by bubbling nitrogen gas into the reaction mixture for 10 min. Finally, Pd-PEPPSI-IHept(C1) (22.14 mg, 22.74 mol) was added and stirred at 110° C. for 16 hours. After completion of the reaction, the reaction mixture was filtered through a Celite pad and filtrate was evaporated under reduced pressure to get crude compound which was purified by preparative HPLC [Column: X-BRIDGE C18 (250 mm*19 mm*5 μm), Mobile phase A: 10 mm NH₄HCO₃ in milli Q-water; Mobile phase B: acetonitrile]. Fractions were collected and lyophilized to afford 6-[2-cyano-3-[[ethyl(methyl)sulfamoyl]amino]-6-fluoro-phenoxy]-3-[7-[1-[3-(2,4-dioxohexahydropyrimidin-1-yl)-5-fluoro-1-methyl-indazol-6-yl]-3,3-difluoro-4-piperidyl]-7-azaspiro[3.5]nonan-2-yl]-4-oxo-quinazoline (Compound 253, 7 mg, 7.57 mol, 7% yield) as an off-white solid. UPLC-MS (ES+): m/z 920.2 [M+H]⁺. ¹H NMR (400 MHz, DMSO-d₆): δ 10.54 (s, 1H), 10.17 (bs, 1H), 8.41 (s, 1H), 7.79 (d, J=8.80 Hz, 2H), 7.67 (dd, J=2.80, 9.00 Hz, 1H), 7.47 (d, J=5.60 Hz, 1H), 7.37-7.34 (m, 2H), 7.18 (d, J=7.20 Hz, 1H), 4.94-4.84 (m, 1H), 3.95 (s, 3H), 3.89 (t, J=6.8 Hz, 2H), 3.69-3.53 (m, 2H), 3.29-2.99 (m, 5H), 2.93-2.83 (m, 1H), 2.80-2.70 (m, 6H), 2.71-2.65 (m, 2H), 2.34-2.31 (m, 2H), 2.27-2.18 (m, 2H), 2.10-2.00 (m, 1H), 1.90-1.80 (m, 1H), 1.70-1.40 (m, 4H), 1.05 (t, J=7.20 Hz, 3H).

### Synthesis D150: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-2-azaspiro[3.3]heptane-2-sulfonamide (Compound 254)

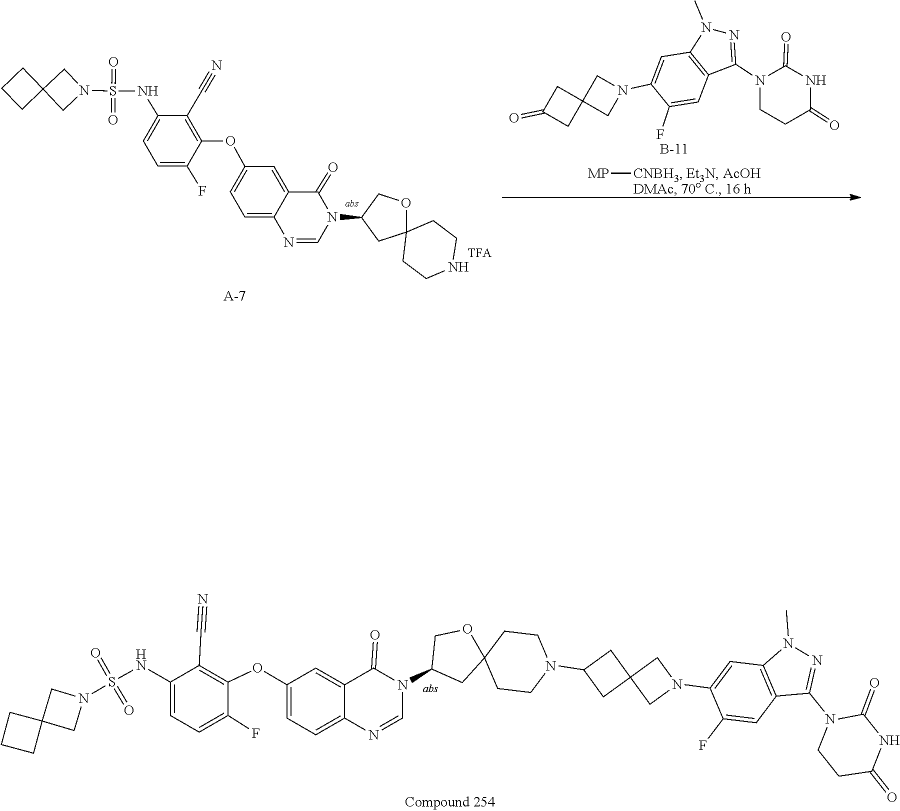

Into a 20 mL pressure vial containing a well-stirred solution of N-[2-cyano-4-fluoro-3-[3-[(3R)-1-oxa-8-azaspiro[4.5]decan-3-yl]-4-oxo-quinazolin-6-yl]oxy-phenyl]-2-azaspiro[3.3]heptane-2-sulfonamide (A-7) (90.00 mg, 73.66 mol, TFA salt) in DMAc (2 mL) was added triethylamine (73.66 mol, 10.27 μL) and stirred for 10 min. Subsequently, AcOH (220.97 mol, 12.63 μL) and 1-[5-fluoro-1-methyl-6-(6-oxo-2-azaspiro[3.3]heptan-2-yl)indazol-3-yl]hexahydropyrimidine-2,4-dione (B-11) (39.08 mg, 73.66 mol) were added and the reaction mixture was stirred at room temperature for 4 hours. Finally, MP-CNBH₃ (90 mg, 73.66 mol) was added, and the reaction mixture was stirred at 70° C. for 16 hours. After completion of the reaction, the reaction mixture was filtered through celite and concentrated under reduced pressure to get a crude residue which was purified by reverse phase purification using the method: [Column: X-BRIDGE C18 (250 mm*19 mm*5 μm); Mobile phase A: 10 mM NH₄HCO₃ in milli-Q water; Mobile phase B: acetonitrile]. Eluted fractions were lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-2-azaspiro[3.3]heptane-2-sulfonamide (Compound 254, 23.86 mg, 24.04 mol, 33% yield) as a white solid. UPLC-MS (ES+): m/z 950.3 [M+H]⁺. ¹H NMR (400 MHz, DMSO-d₆): δ 10.50 (s, 1H), 8.31 (s, 1H), 7.76 (d, J=8.80 Hz, 1H), 7.64 (dd, J=3.20, 9.00 Hz, 1H), 7.34-7.25 (m, 4H), 6.52 (d, J=7.20 Hz, 1H), 5.40-5.22 (m, 1H), 4.23-4.08 (m, 2H), 4.07-4.01 (m, 2H), 3.95-3.89 (m, 2H), 3.89-3.83 (m, 5H), 3.70-3.49 (m, 5H), 2.79-2.71 (m, 2H), 2.45-2.36 (m, 2H), 2.17-2.10 (m, 1H), 2.09-1.99 (m, 5H), 1.92-1.82 (m, 2H), 1.81-1.67 (m, 3H).

### Synthesis D151: Synthesis of 6-[2-cyano-3-[[ethyl(methyl)sulfamoyl]amino]-6-fluoro-phenoxy]-3-[7-[[1-[3-(2,4-dioxohexahydropyrimidin-1-yl)-5-fluoro-1-methyl-indazol-6-yl]-4-piperidyl]methyl]-7-azaspiro[3.5]nonan-2-yl]-4-oxo-quinazoline (Compound 255)

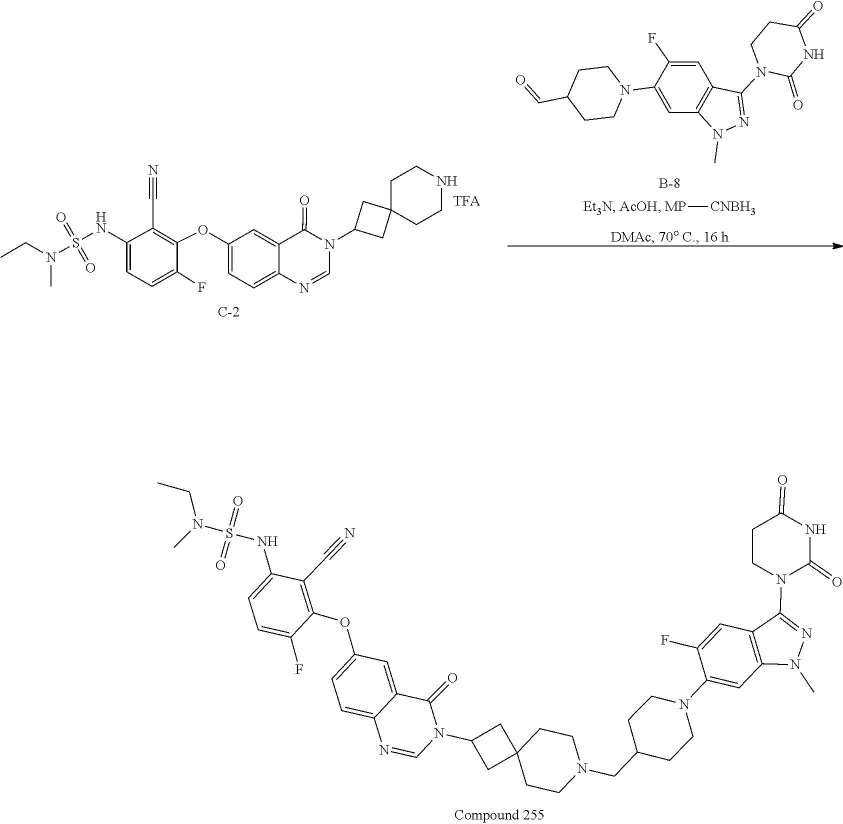

Into a 8 mL pressure vial containing a well-stirred solution of 1-[3-(2,4-dioxohexahydropyrimidin-1-yl)-5-fluoro-1-methyl-indazol-6-yl]piperidine-4-carbaldehyde (B-8) (50 mg, 133.91 mol) and 3-(7-azaspiro[3.5]nonan-2-yl)-6-[2-cyano-3-[[ethyl(methyl)sulfamoyl]amino]-6-fluoro-phenoxy]-4-oxo-quinazoline (C-2) (87.66 mg, 133.91 mol, TFA salt) in anhydrous DMAc (2.0 mL) was added triethylamine (535.65 mol, 74.66 μL) followed by AcOH (267.82 mol, 15.31 μL) and stirred for 2 hours. Subsequently, MP-CNBH₃ (70 mg, 133.91 mol) was added to the reaction mixture and stirred at 70° C. for 16 hours. After completion of the reaction, the reaction mixture was diluted with dichloromethane (20 mL), filtered through cotton pad and filtrate was concentrated under reduced pressure to get crude, which was purified by prep HPLC by using Method: [X-Bridge C18, (150 mm×19 mm×5 μm); Mobile phase A: 10 mM NH₄HCO₃ (pH:10 adjust with Ammonia) in water, Mobile phase B: acetonitrile, Flow: 15 mL/min] Eluted fraction were lyophilized to afford 6-[2-cyano-3-[[ethyl(methyl)sulfamoyl]amino]-6-fluoro-phenoxy]-3-[7-[[1-[3-(2,4-dioxohexahydropyrimidin-1-yl)-5-fluoro-1-methyl-indazol-6-yl]-4-piperidyl]methyl]-7-azaspiro[3.5]nonan-2-yl]-4-oxo-quinazoline (Compound 255, 11 mg, 12.16 mol, 9% yield) as an off-white solid. UPLC-MS (ES+): m/z 898.2 [M+H]⁺. ¹H NMR (400 MHz, DMSO-d₆): δ 10.53 (s, 1H), 8.39 (s, 1H), 7.77 (d, J=8.80 Hz, 1H), 7.65-7.62 (m, 1H), 7.46 (bs, 1H), 7.37-7.34 (m, 3H), 7.09 (d, J=7.20 Hz, 1H), 4.98-4.91 (m, 1H), 3.95 (s, 3H), 3.90 (t, J=6.80 Hz, 2H), 3.53-3.46 (m, 2H), 3.48-3.45 (m, 2H), 3.09-3.06 (m, 1H), 2.82-2.72 (m, 4H), 2.66-2.62 (m, 3H), 2.69-2.62 (m, 4H), 2.34-2.33 (m, 6H), 1.92-1.72 (m, 6H), 1.42-1.35 (m, 2H), 1.03 (t, J=7.20 Hz, 3H).

### Synthesis D152: Synthesis of 7-[2-cyano-3-[[ethyl(methyl)sulfamoyl]amino]-6-fluoro-phenoxy]-2-[7-[1-[3-(2,4-dioxohexahydropyrimidin-1-yl)-5-fluoro-1-methyl-indazol-6-yl]-4-piperidyl]-7-azaspiro[3.5]nonan-2-yl]quinoxaline (Compound 256)

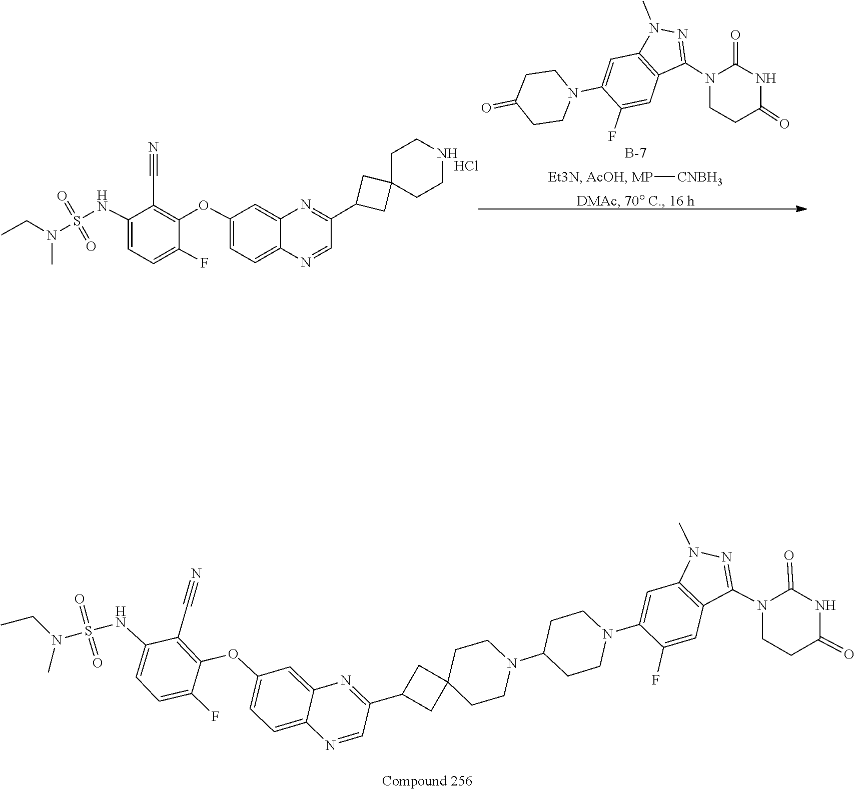

Into a 40 mL pressure vial containing a well stirred solution 2-(7-azaspiro[3.5]nonan-2-yl)-7-[2-cyano-3-[[ethyl(methyl)sulfamoyl]amino]-6-fluoro-phenoxy]quinoxaline (A-46) (200 mg, 238.83 mol, HCl salt) and 1-[5-fluoro-1-methyl-6-(4-oxo-1-piperidyl)indazol-3-yl]hexahydropyrimidine-2,4-dione (B-7) (107.28 mg, 262.71 mol) in anhydrous DMAc (4.0 mL) was added triethylamine (716.49 mol, 99.86 μL) followed by AcOH (477.66 mol, 27.31 μL) and stirred at room temperature for 1 hours. Finally, MP-CNBH₃ (150 mg, 238.83 mol) was added and stirred at 70° C. for 16 hours. After completion of the reaction, the reaction mixture was diluted with DCM (10 mL) and filtered, filtrate was concentrated under reduced pressure to afford crude residue which was purified by preparative HPLC [Column: X-Bridge C8 (150 mm*19 mm*5 μm), Mobile phase A: 10 mM NH₄HCO₃ & NH₄OH_pH-10 in milli-Q water; Mobile phase B: acetonitrile]. The fraction was lyophilized to afford 7-[2-cyano-3-[[ethyl(methyl)sulfamoyl]amino]-6-fluoro-phenoxy]-2-[7-[1-[3-(2,4-dioxohexahydropyrimidin-1-yl)-5-fluoro-1-methyl-indazol-6-yl]-4-piperidyl]-7-azaspiro[3.5]nonan-2-yl]quinoxaline (Compound 256, 10 mg, 11.13 mol, 5% yield) as an off-white solid. UPLC-MS (ES+): m/z 868.5 [M+H]⁺. ¹H NMR (400 MHz, DMSO-d₆): δ 10.54 (s, 1H), 9.25 (bs, 1H), 8.78 (s, 1H), 8.14-8.11 (m, 1H), 7.68 (dd, J=2.80, 9.20 Hz, 1H), 7.48-7.26 (m, 3H), 7.14 (d, J=6.80 Hz, 1H), 7.10 (d, J=2.40 Hz, 1H), 3.96 (s, 3H), 3.93-3.92 (m, 1H), 3.90 (t, J=6.80 Hz, 2H), 3.62-3.53 (m, 2H), 3.30-3.10 (m, 2H), 3.01 (q, J=7.20 Hz, 2H), 2.98-2.80 (m, 2H), 2.78-2.71 (m, 4H), 2.59 (s, 3H), 2.32-2.20 (m, 3H), 2.15-1.55 (m, 8H), 1.04 (t, J=7.20 Hz, 3H)

### Synthesis D154: Synthesis of 6-[2-cyano-3-[[ethyl(methyl)sulfamoyl]amino]-6-fluoro-phenoxy]-3-[[1-[2-[3-(2,4-dioxohexahydropyrimidin-1-yl)-5-fluoro-1-methyl-indazol-6-yl]-2-azaspiro[3.3]heptan-6-yl]-4-piperidyl]methyl]-4-oxo-quinazoline (Compound 258)

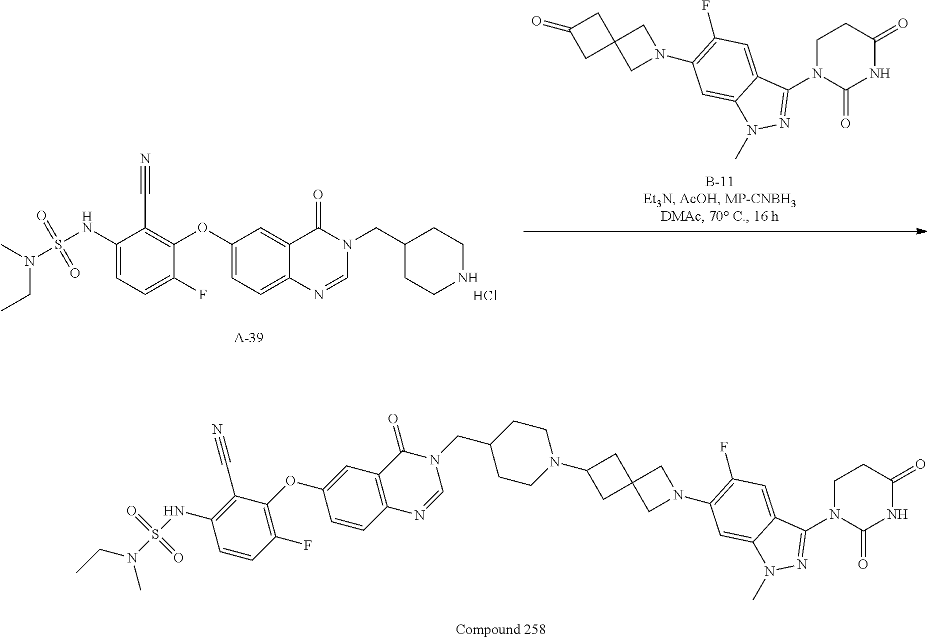

Into a 20 mL pressure vial containing a well-stirred solution of 6-[2-cyano-3-[[ethyl(methyl)sulfamoyl]amino]-6-fluoro-phenoxy]-4-oxo-3-(4-piperidylmethyl)quinazoline (A-39) (120 mg, 191.64 mol, HCl salt) in anhydrous DMAc (4.01 mL) was added triethylamine (958.20 mol, 133.55 μL) and stirred for 30 min. Subsequently, 1-[5-fluoro-1-methyl-6-(6-oxo-2-azaspiro[3.3]heptan-2-yl)indazol-3-yl]hexahydropyrimidine-2,4-dione (B-11) (87.86 mg, 191.64 mol) and AcOH (574.92 mol, 34.52 μL) were added and stirred for 1 hour. Finally, MP-CNBH₃ (120 mg, 19.43 mol) was added and resulting reaction mixture was stirred at 70° C. for 16 hours. After completion of the reaction, the reaction mixture was filtered through sintered funnel and the filtrate was concentrated under reduced pressure to get crude residue which was purified by reverse phase purification [Column: Redisep R_{f}Gold® reversed-phase C18-teledyne ISCO, 120), [Mobile phase A: 10 mM NH₄HCO₃ in milli-Q-water; Mobile phase B: acetonitrile]. Fractions were collected and lyophilized, resulting compound was impure. Further re-purified by preparative HPLC [Column: X-Select C18 (250 mm*19 mm*5 μm), Mobile phase A: 10 mM NH₄HCO₃ in milli-Q water; Mobile phase B: acetonitrile]. Fractions were collected and lyophilized to afford 6-[2-cyano-3-[[ethyl(methyl)sulfamoyl]amino]-6-fluoro-phenoxy]-3-[[1-[2-[3-(2,4-dioxohexahydropyrimidin-1-yl)-5-fluoro-1-methyl-indazol-6-yl]-2-azaspiro[3.3]heptan-6-yl]-4-piperidyl]methyl]-4-oxo-quinazoline (Compound 258, 39 mg, 44.72 mol, 23% yield) as an off-white solid. UPLC-MS (ES+): m/z 870.2 [M+H]⁺. ¹H NMR (400 MHz, DMSO-d₆): δ 10.50 (s, 1H), 8.34 (s, 1H), 7.78 (d, J=8.80 Hz, 1H), 7.65 (dd, J=3.20, 8.80 Hz, 1H), 7.55-7.43 (m, 1H), 7.34-7.24 (m, 3H), 6.50 (d, J=7.60 Hz, 1H), 4.01 (s, 2H), 3.95-3.85 (m, 10H), 3.20-3.10 (m, 2H), 3.03 (t, J=7.20 Hz, 2H), 2.74 (t, J=6.80 Hz, 2H), 2.59 (s, 3H), 2.57-2.54 (m, 2H), 2.48-2.40 (m, 2H), 2.30-2.15 (m, 2H), 2.10-1.90 (m, 1H), 1.80-1.65 (m, 2H), 1.45-1.22 (m, 2H), 1.00 (t, J=7.20 Hz, 3H).

### Synthesis D155: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]cyclobutanesulfonamide (Compound 259)

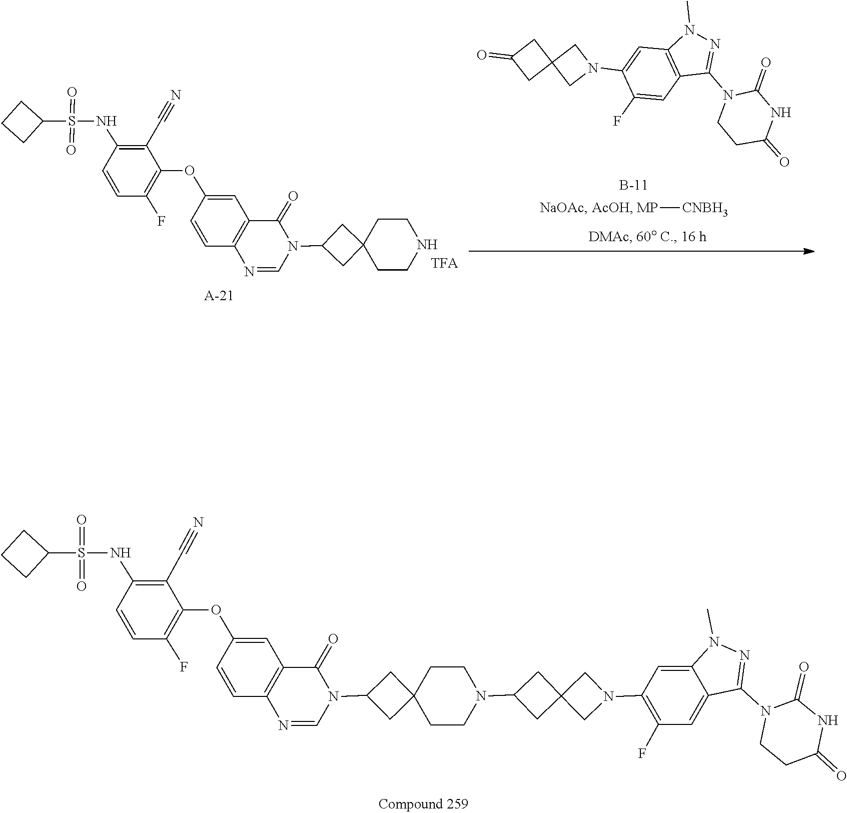

Into a 20 mL pressure vial containing a well stirred solution of N-[3-[3-(7-azaspiro[3.5]nonan-2-yl)-4-oxo-quinazolin-6-yl]oxy-2-cyano-4-fluoro-phenyl]cyclobutanesulfonamide (A-21) (0.220 g, 337.62 mol, TFA salt) and 1-[5-fluoro-1-methyl-6-(6-oxo-2-azaspiro[3.3]heptan-2-yl)indazol-3-yl]hexahydropyrimidine-2,4-dione 2 (B-11) (62.69 mg, 168.81 mol) in DMAc (5 mL) was added NaOAc (138.47 mg, 1.69 mmol) and resulting mixture was stirred at room temperature for 1 hour. After 1 h, AcOH (1.01 mmol, 57.92 L) was added and stirred for 2 hours. Subsequently, MP-CNBH₃ (0.330 g) was added and resulting reaction mixture was stirred at 60° C. for 16 hours. After completion of the reaction, the reaction mixture was filtered through sintered funnel and filtrate was concentrated under reduced pressure to get a crude residue which was purified by the reverse phase purification [Column: Redisep R_{f} Gold® C18-teledyne ISCO, 100 g), Mobile phase A: 10 mM NH₄HCO₃ in milli-Q water; Mobile phase B: acetonitrile]. Fractions were collected and lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]cyclobutanesulfonamide (Compound 259, 0.046 g, 51.18 mol, 15% yield) as a white solid. UPLC-MS (ES+): m/z 893.2 [M+H]⁺. ¹H NMR (400 MHz, DMSO-d₆): δ 10.51 (s, 1H), 9.50 (bs, 1H), 8.39 (s, 1H), 7.76 (d, J=8.80 Hz, 1H), 7.63 (dd, J=2.80, 9.00 Hz, 1H), 7.55-7.40 (m, 1H), 7.36-7.26 (m, 3H), 6.52 (d, J=7.20 Hz, 1H), 5.00-4.90 (m, 1H), 4.02 (s, 2H), 3.96-3.86 (m, 8H), 3.80-3.68 (m, 2H), 2.90-2.71 (m, 5H), 2.65-2.55 (m, 2H), 2.46-2.38 (m, 4H), 2.30-2.21 (m, 3H), 2.20-2.10 (m, 2H), 1.92-1.60 (m, 7H).

### Synthesis D156: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]cyclopropanesulfonamide (Compound 260)

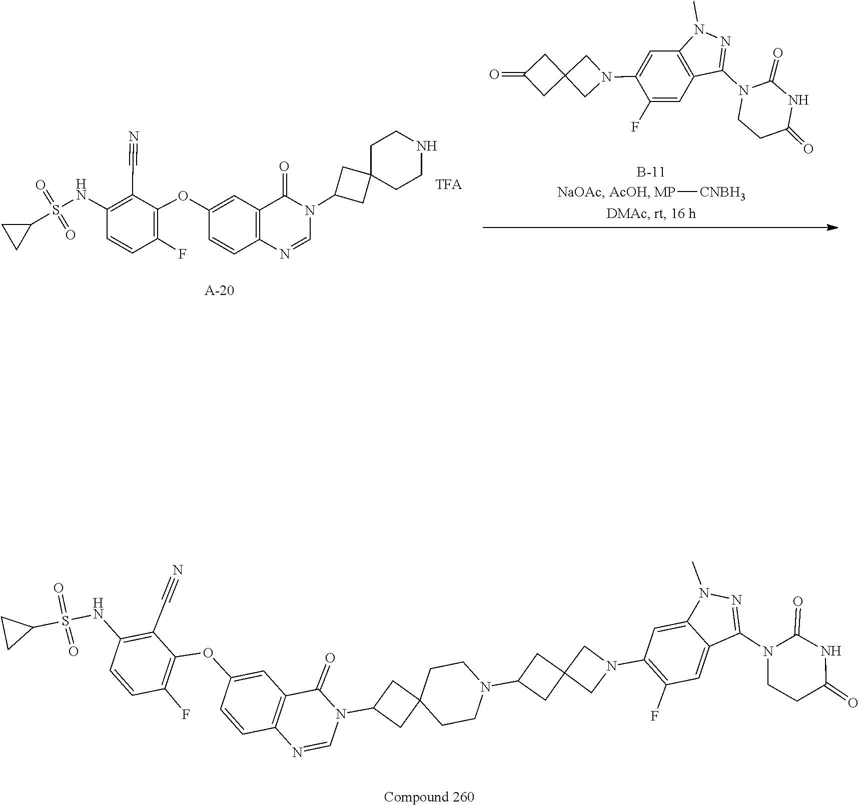

Into a 20 mL glass vial with pressure released cap containing a well-stirred solution of N-[3-[3-(7-azaspiro[3.5]nonan-2-yl)-4-oxo-quinazolin-6-yl]oxy-2-cyano-4-fluoro-phenyl]cyclopropanesulfonamide (A-20) (150 mg, 197.62 mol, TFA salt) in DMAc (5 mL) was added NaOAc (81.05 mg, 988.08 mol) at room temperature and stirred for 10 min. Subsequently, AcOH (592.85 mol, 33.90 μL) and 1-[5-fluoro-1-methyl-6-(6-oxo-2-azaspiro[3.3]heptan-2-yl)indazol-3-yl]hexahydropyrimidine-2,4-dione (B-11) (93.87 mg, 217.38 mol) were added at room temperature and stirred for 2 hours. Finally, MP-CNBH₃ (150 mg, 197.62 mol) was added and stirred at room temperature for 16 hours. After completion of the reaction, the reaction mixture was filtered on cotton pad and filtrate was concentrated under reduced pressure to get crude residue, which was purified by reverse phase column using method [Column: (Redisep R_{f} Gold®reversed-phase C18-teledyne ISCO, 120 g), Mobile phase A: 10 mM NH₄HCO₃ in milli-Q water; Mobile phase B: acetonitrile]. Collected fractions were dried to afford 40 mg of desired product, which is not pure. Further purified by prep HPLC by using a method [Column: X-Bridge C18 (150 mm×19 mm×5 μm) and Mobile phase A: 10 mM NH₄HCO₃ (pH-10 adjusted with NH₄OH) in milli-Q water; Mobile phase B: acetonitrile]. Collected fractions were lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]cyclopropanesulfonamide (Compound 260, 18 mg, 20.04 mol, 10% yield) as an off-white solid. UPLC-MS (ES+): m/z 879.3 [M+H]⁺. ¹H-NMR (400 MHz, DMSO-d₆): δ 10.50 (s, 1H), 9.53 (bs, 1H), 8.39 (s, 1H), 7.77 (d, J=8.80 Hz, 1H), 7.64 (dd, J=3.20, 9.00 Hz, 1H), 7.50 (bs, 1H), 7.36-7.26 (m, 3H), 6.52 (d, J=7.60 Hz, 1H), 5.09-4.87 (m, 1H), 4.03-3.86 (m, 10H), 3.37-2.67 (m, 5H), 2.60-2.50 (m, 2H), 2.36-2.33 (m, 8H), 1.92-1.76 (m, 4H), 0.85 (d, J=4.80 Hz, 4H).

### Synthesis D157: Synthesis of (3R)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-fluoro-pyrrolidine-1-sulfonamide (Compound 261)

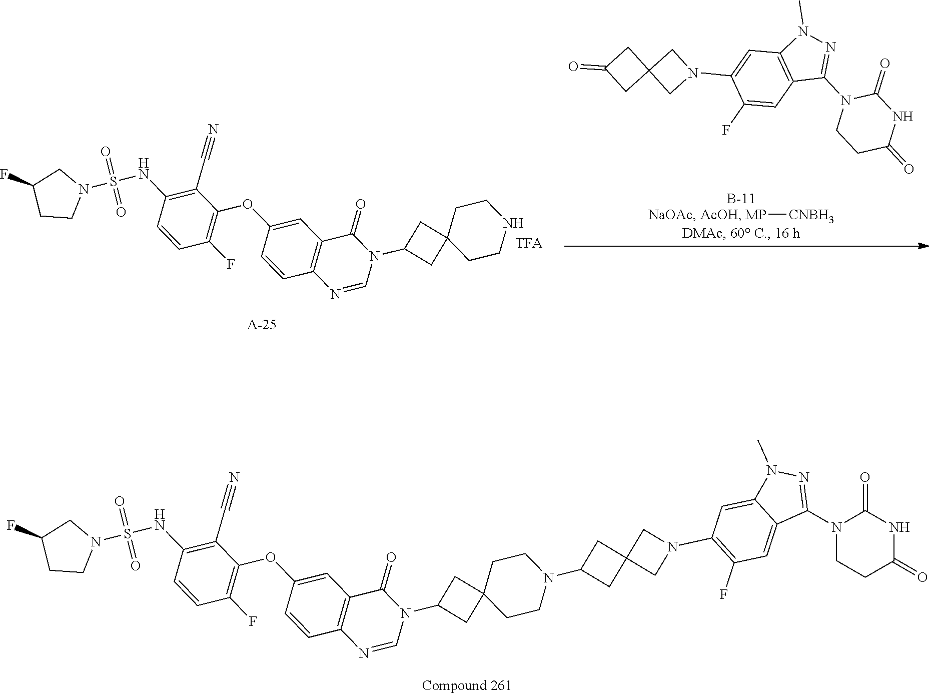

Into a 20 mL pressure vial containing a well stirred solution of (3R)-N-[3-[3-(7-azaspiro[3.5]nonan-2-yl)-4-oxo-quinazolin-6-yl]oxy-2-cyano-4-fluoro-phenyl]-3-fluoro-pyrrolidine-1-sulfonamide (A-25) (0.250 g, 365.16 mol, TFA salt) and 1-[5-fluoro-1-methyl-6-(6-oxo-2-azaspiro[3.3]heptan-2-yl)indazol-3-yl]hexahydropyrimidine-2,4-dione (B-11) (67.80 mg, 182.58 mol) in DMAc (5 mL) was added NaOAc (179.72 mg, 2.19 mmol) and resulting mixture was stirred at room temperature for 1 hour. After 1 h, AcOH (1.31 mmol, 75.18 μL) was added and stirred at room temperature for 2 hours. Subsequently, MP-CNBH₃ (0.400 g) was added and stirred at 60° C. for 16 hours. After completion the reaction, reaction mixture was filtered through sintered funnel and filtrate was concentrated under reduced pressure to get a crude residue which was purified by the reverse phase purification [Column: Redisep R_{f} Gold® C18-teledyne ISCO, 100 g), Mobile phase A: 10 mM NH₄HCO₃ in milli-Q water; Mobile phase B: acetonitrile]. Fractions were collected and lyophilized to afford mixture of cis and trans isomers. Further cis and trans isomers were separated by preparative HPLC [Column: Shimpack C18 (150 mm*19 mm*5 μm), Mobile phase A: 10 mM NH₄HCO₃ in milli-Q water (pH was adjusted to 10 with NH₄OH); Mobile phase B: acetonitrile]. Fractions were collected and lyophilized to afford (3R)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-fluoro-pyrrolidine-1-sulfonamide (Compound 261, 0.033 g, 35.01 mol, 10% yield) as a white solid. UPLC-MS (ES+): m/z 926.2 [M+H]⁺. ¹H NMR (400 MHz, DMSO-d₆): δ 10.51 (s, 1H), 9.28 (bs, 1H), 8.38 (s, 1H), 7.76 (d, J=8.80 Hz, 1H), 7.63 (dd, J=3.20, 9.00 Hz, 1H), 7.43-7.26 (m, 4H), 6.53 (d, J=7.20 Hz, 1H), 5.35-5.20 (m, 1H), 5.00-4.90 (m, 1H), 4.03-3.86 (m, 10H), 3.50-3.38 (m, 2H), 3.27-3.15 (m, 3H), 2.90-2.71 (m, 5H), 2.62-2.58 (m, 2H), 2.41-2.30 (m, 5H), 2.20-1.65 (m, 7H).

### Synthesis D158: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]morpholine-4-sulfonamide (Compound 262)

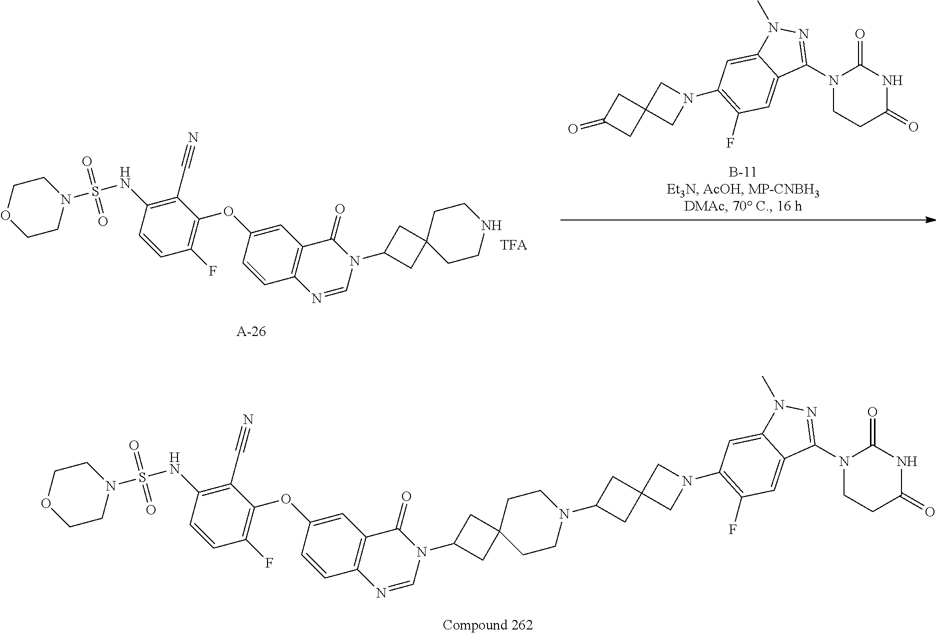

Into a 20 mL pressure vial containing a well stirred solution of N-[3-[3-(7-azaspiro[3.5]nonan-2-yl)-4-oxo-quinazolin-6-yl]oxy-2-cyano-4-fluoro-phenyl]morpholine-4-sulfonamide (A-26) (240 mg, 297.96 mol, TFA salt) in DMAc (4 mL) was added triethylamine (893.88 mol, 124.59 μL) and stirred for 30 min at room temperature. Subsequently, AcOH (893.88 mol, 51.12 μL) and 1-[5-fluoro-1-methyl-6-(6-oxo-2-azaspiro[3.3]heptan-2-yl)indazol-3-yl]hexahydropyrimidine-2,4-dione (B-11) (160.95 mg, 357.55 mol) were added and resulting mixture was stirred for 3 hr at room temperature. Finally, MP-CNBH₃ (260 mg, 336.70 mol) was added and stirred at 70° C. for 16 hours. After completion of the reaction, the reaction mixture was filtered through cotton pad and filtrate was concentrated under reduced pressure to afford crude residue, which was purified by preparative HPLC [Column: X-Bridge C18 (250 mm*19 mm*5 m), Mobile phase A: 10 mM NH₄HCO₃ and NH₄OH (pH-10); Mobile phase B: acetonitrile, Flow rate: 14 mL/min]. Fractions were concentrated under reduced pressure at 40° C. and lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]morpholine-4-sulfonamide (Compound 262, 57.17 mg, 61.35 mol, 21% yield) as a white solid. UPLC-MS (ES+): m/z 924.20 [M+H]⁺. ¹H NMR (400 MHz, DMSO-d₆): δ 10.51 (bs, 1H), 9.12 (s, 1H), 8.38 (s, 1H), 7.76 (d, J=8.80 Hz, 1H), 7.64-7.61 (m, 1H), 7.37-7.26 (m, 4H), 6.53 (d, J=7.60 Hz, 1H), 5.12-4.87 (m, 1H), 4.03 (s, 2H), 3.94 (s, 2H), 3.89-3.81 (m, 5H), 3.62-3.51 (m, 4H), 3.29-3.04 (m, 2H), 2.94 (s, 4H), 2.73 (t, J=6.80 Hz, 3H), 2.68-2.66 (m, 2H), 2.56-2.54 (m, 3H), 2.48-2.33 (m, 5H), 2.16-1.92 (m, 2H), 1.84-1.52 (m, 2H).

### Synthesis D159: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]tetrahydropyran-4-sulfonamide (Compound 263)

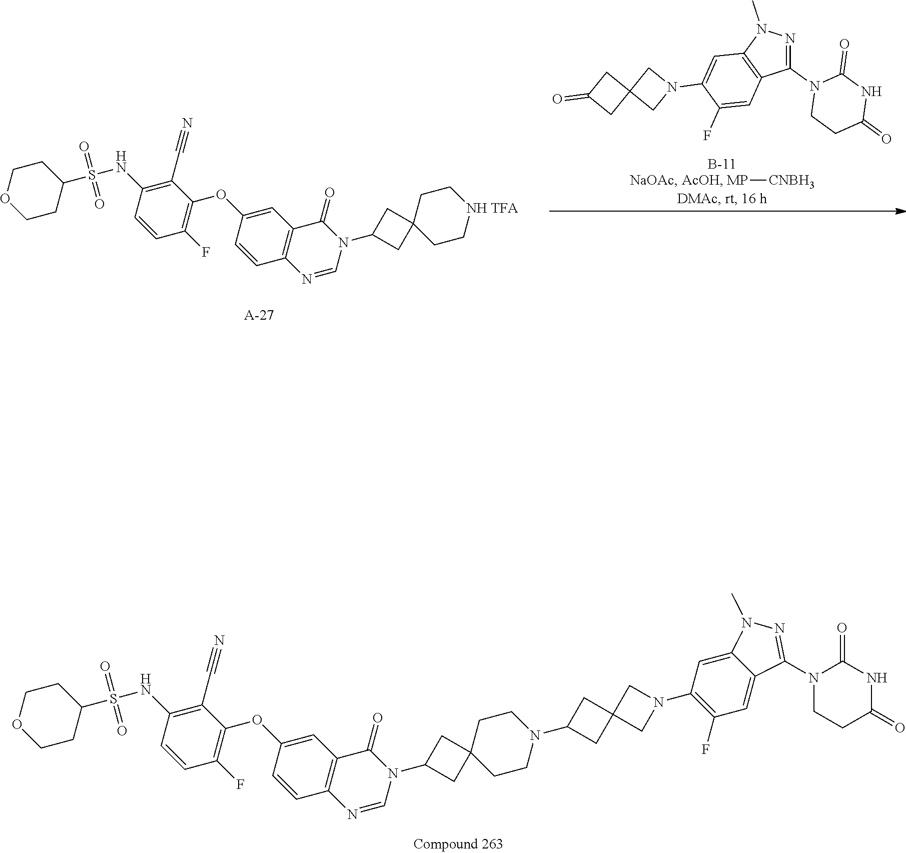

Into a 20 mL glass vial with pressure released cap containing a well-stirred solution of N-[3-[3-(7-azaspiro[3.5]nonan-2-yl)-4-oxo-quinazolin-6-yl]oxy-2-cyano-4-fluoro-phenyl]tetrahydropyran-4-sulfonamide (A-27) (150 mg, 187.05 mol, TFA salt) in DMAc (5 mL) was added NaOAc (76.72 mg, 935.23 mol) at room temperature and stirred for 10 min. Subsequently, AcOH (561.14 mol, 32.09 μL) and 1-[5-fluoro-1-methyl-6-(6-oxo-2-azaspiro[3.3]heptan-2-yl)indazol-3-yl]hexahydropyrimidine-2,4-dione (B-11) (80.77 mg, 187.05 mol,) were added at room temperature and stirred for 2 hours. Finally, MP-CNBH₃ (150 mg, 187.05 mol) was added and stirred at room temperature for 16 hours. After completion of the reaction, the reaction mixture was filtered on cotton pad and filtrate was concentrated under reduced pressure to get crude residue, which was purified by prep HPLC by using a method [Column: X-Bridge C18 (150 mm×19 mm×5 μm) and Mobile phase A:10 mM NH₄HCO₃ (pH-10 adjusted with NH₄OH) in milli-Q water; Mobile phase B: acetonitrile]. Collected fractions were lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]tetrahydropyran-4-sulfonamide (Compound 263, 19 mg, 20.53 mol, 11% yield) as an off-white solid. UPLC-MS (ES+): m/z 923.2 [M+H]⁺. ¹H NMR (400 MHz, DMSO-d₆): δ 10.51 (s, 1H), 9.21 (bs, 1H), 8.38 (s, 1H), 7.76 (d, J=9.20 Hz, 1H), 7.63 (dd, J=3.20, 9.00 Hz, 1H), 7.35-7.26 (m, 4H), 6.53 (d, J=7.20 Hz, 1H), 5.07-4.91 (m, 1H), 4.03-3.86 (m, 11H), 3.60-3.50 (m, 2H), 3.32-3.23 (m, 2H), 2.97-2.67 (m, 5H), 2.60-2.55 (m, 2H), 2.51-2.33 (m, 6H), 1.91-1.61 (m, 9H).

### Synthesis D160: Synthesis of 6-[2-cyano-3-[[ethyl(methyl)sulfamoyl]amino]-6-fluoro-phenoxy]-3-[[1-[1-[3-(2,4-dioxohexahydropyrimidin-1-yl)-5-fluoro-1-methyl-indazol-6-yl]-4-piperidyl]-4-piperidyl]methyl]-4-oxo-quinazoline (Compound 264)

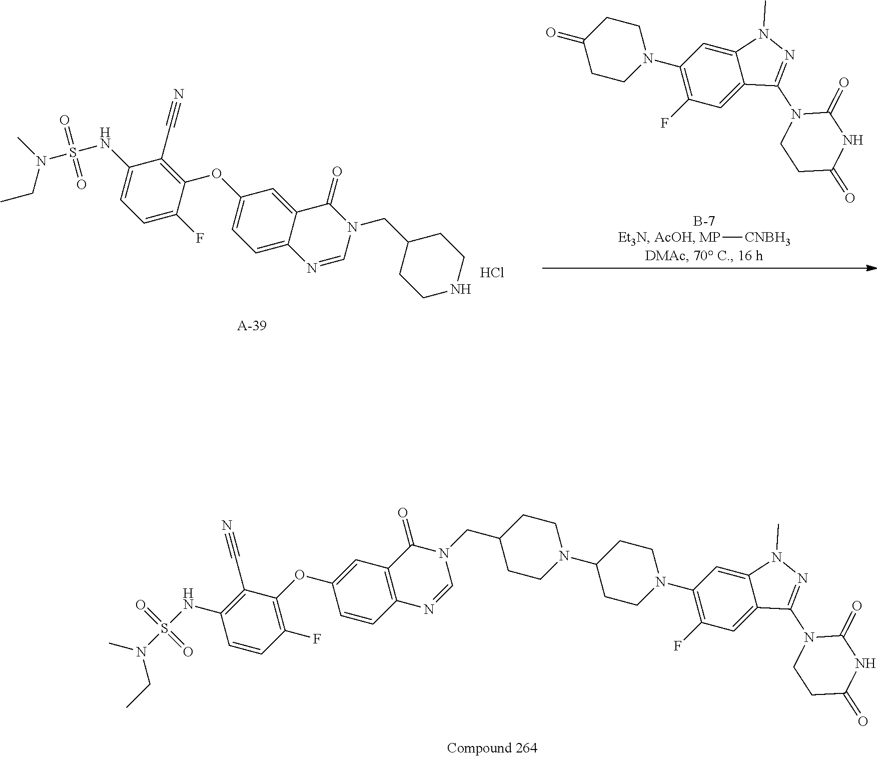

Into a 20 mL pressure vial containing a well-stirred solution of 6-[2-cyano-3-[[ethyl(methyl)sulfamoyl]amino]-6-fluoro-phenoxy]-4-oxo-3-(4-piperidylmethyl)quinazoline (A-39) (150 mg, 239.55 mol, HCl Salt) in anhydrous DMAc (3.03 mL) was added triethylamine (1.20 mmol, 166.94 μL) and stirred for 30 min. After 30 min, 1-[5-fluoro-1-methyl-6-(4-oxo-1-piperidyl)indazol-3-yl]hexahydropyrimidine-2,4-dione (B-7) (100.10 mg, 239.55 mol) and AcOH (718.65 mol, 43.16 μL) were added and stirred for 2 hours. Subsequently, MP-CNBH₃ (150 mg) was added and resulting reaction mixture was stirred at 70° C. for 16 hours. After completion of the reaction, the reaction mixture was filtered through sintered funnel and filtrate was concentrated under reduced pressure to get crude residue which was purified by reverse phase column chromatography [Column: Redisep R_{f} Gold® reversed-phase C18-teledyne ISCO, 120 g), Mobile phase A: 10 mM NH₄HCO₃ in milli-Q water; Mobile phase B: acetonitrile]. Fractions were collected and lyophilized, resulting compound was impure. Further, re-purified by preparative HPLC [Column: X-Select C18 (250 mm*19 mm*5 μm), Mobile phase A: 10 mM NH₄HCO₃ in milli-Q water; Mobile phase B: acetonitrile]. Fractions were collected and lyophilized to afford 6-[2-cyano-3-[[ethyl(methyl)sulfamoyl]amino]-6-fluoro-phenoxy]-3-[[1-[1-[3-(2,4-dioxohexahydropyrimidin-1l-yl)-5-fluoro-1-methyl-indazol-6-yl]-4-piperidyl]-4-piperidyl]methyl]-4-oxo-quinazoline (Compound 264, 28 mg, 32.55 mol, 14% yield) as an off-white solid. UPLC-MS (ES+): m/z 858.2 [M+H]⁺. ¹H NMR (400 MHz, DMSO-d₆): δ 10.53 (s, 1H), 8.93 (bs, 1H), 8.35 (s, 1H), 7.78 (d, J=8.80 Hz, 1H), 7.65 (dd, J=2.80, 8.80 Hz, 1H), 7.38-7.11 (m, 5H), 3.98-3.87 (m, 7H), 3.65-3.45 (m, 3H), 3.10-2.70 (m, 8H), 2.65-2.58 (m, 4H), 2.20-1.30 (m, 10H), 1.03 (t, J=7.20 Hz, 3H).

### Synthesis D161: Synthesis of (3R)-N-[2-cyano-3-[3-[7-[2-[3-(2,4-dioxohexahydropyrimidin-1-yl)-5-fluoro-1-methyl-indazol-6-yl]-2-azaspiro [3.3]heptan-6-yl]-7-azaspiro [3.5]nonan-2-yl]quinoxalin-6-yl]oxy-4-fluoro-phenyl]-3-fluoro-pyrrolidine-1-sulfonamide (Compound 265)

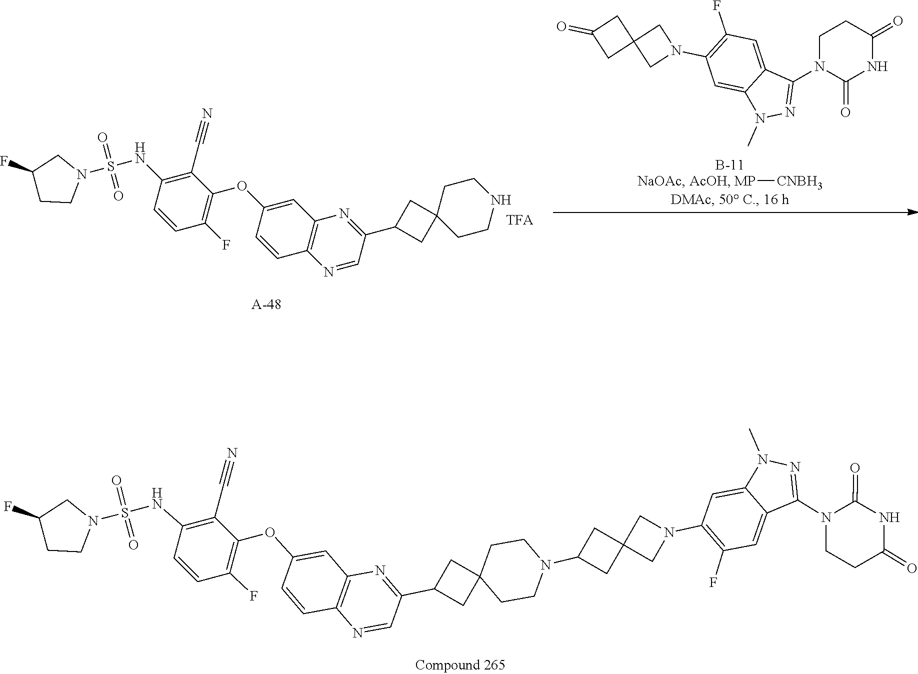

Into a 20 mL reaction vial containing a well stirred solution of (3R)-N-[3-[3-(7-azaspiro[3.5]nonan-2-yl)quinoxalin-6-yl]oxy-2-cyano-4-fluoro-phenyl]-3-fluoro-pyrrolidine-1l-sulfonamide (A-48) (150.00 mg, 224.34 mol, TFA salt) in DMAc (3 mL) was added anhydrous NaOAc (36.80 mg, 448.68 mol) at room temperature and stirred for 10 min. Subsequently, AcOH (673.01 mol, 38.48 μL) and 1-[5-fluoro-1-methyl-6-(6-oxo-2-azaspiro[3.3]heptan-2-yl)indazol-3-yl]hexahydropyrimidine-2,4-dione (B-11) (99.97 mg, 269.21 mol) were added and stirred for 6 h at room temperature. Finally, MP-CNBH₃ (100 mg, 224.34 mol) was added and stirred at 50° C. for 16 hours. After completion of the reaction, the reaction mixture was filtered through celite and filtrate was concentrated under reduced pressure to give a crude residue. The crude was purified by reverse phase column chromatography (Column: Redisep R_{f} Gold® reversed-phase C18-teledyne ISCO, 60 g, Mobile phase A: 10 mM NH₄HCO₃ in milli-Q water; Mobile phase B: acetonitrile). The eluting fraction was lyophilized to get compound (50 mg, Purity: 67%) as an off-white solid. The resultant compound was further purified by preparative HPLC [Column: X-BRIDGE C18 (250 mm×19 mm×5 μm), Mobile phase A: 10 mM NH₄HCO₃ (pH:10 adjusted with ammonia solution) in milli-Q water; Mobile phase B: acetonitrile]. The eluted fraction was collected and lyophilized to afford (3R)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]-3-fluoro-pyrrolidine-1-sulfonamide (Compound 265, 9.78 mg, 10.11 mol, 5% yield) as an off-white solid. UPLC-MS (ES+): m/z 910.3 [M+H]⁺. ¹H NMR (400 MHz, DMSO-d₆): δ 10.51 (s, 1H), 9.14 (s, 1H), 8.77 (s, 1H), 8.14-8.11 (m, 1H), 7.68 (dd, J=2.80, 9.20 Hz, 1H), 7.43-7.26 (m, 3H), 7.10 (d, J=2.00 Hz, 1H), 6.53 (d, J=7.20 Hz, 1H), 5.35-5.21 (m, 1H), 4.03 (s, 2H), 3.94-3.86 (m, 9H), 3.39-3.37 (m, 1H), 3.27-3.16 (m, 4H), 2.73 (t, J=6.80 Hz, 3H), 2.46-2.13 (m, 10H), 2.10-1.50 (m, 6H).

### Synthesis D162: Synthesis of 6-[2-cyano-3-[[ethyl(methyl)sulfamoyl]amino]-6-fluoro-phenoxy]-3-[6-[7-[3-(2,4-dioxohexahydropyrimidin-1-yl)-5-fluoro-1-methyl-indazol-6-yl]-7-azaspiro[3.5]nonan-2-yl]-6-azaspiro[3.4]octan-2-yl]-4-oxo-quinazoline (Compound 266)

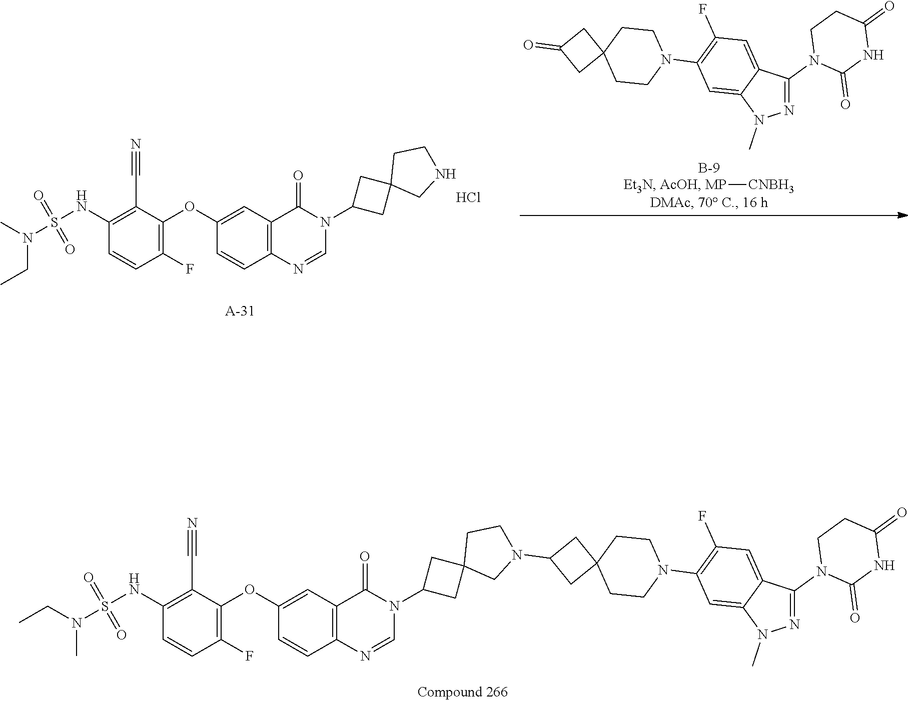

Into a 20 mL pressure vial containing a well-stirred solution of 3-(6-azaspiro[3.4]octan-2-yl)-6-[2-cyano-3-[[ethyl(methyl)sulfamoyl]amino]-6-fluoro-phenoxy]-4-oxo-quinazoline (A-31) (228.26 mg, 355.21 mol, HCl salt) in anhydrous DMAc (4.5 mL) was added triethylamine (1.78 mmol, 247.55 μL) at room temperature under nitrogen atmosphere and mixture was stirred for 20 min. Reaction mixture was cooled to 0° C. and was added 1-[5-fluoro-1-methyl-6-(2-oxo-7-azaspiro[3.5]nonan-7-yl)indazol-3-yl]hexahydropyrimidine-2,4-dione (B-9) (99.31 mg, 248.65 mol) followed by AcOH (1.07 mmol, 60.95 μL) and stirred for 3 h at room temperature. Subsequently, MP-CNBH₃ (500 mg, 355.21 mol) was added, and resulting reaction mixture was stirred at 70° C. for 16 hours. After the completion of the reaction, the reaction mixture was filtered through a cotton pad and the filtrate was concentrated under reduced pressure to get a crude residue which was purified by reverse phase column chromatography (Column: Redisep R_{f} Gold® reversed-phase C18-teledyne ISCO, 120 g), [Mobile phase A: 10 mM NH₄HCO₃ in milli-Q water; Mobile phase B: acetonitrile]. The fractions were collected and lyophilized to afford 6-[2-cyano-3-[[ethyl(methyl)sulfamoyl]amino]-6-fluoro-phenoxy]-3-[6-[7-[3-(2,4-dioxohexahydropyrimidin-1-yl)-5-fluoro-1-methyl-indazol-6-yl]-7-azaspiro[3.5]nonan-2-yl]-6-azaspiro[3.4]octan-2-yl]-4-oxo-quinazoline (Compound 266, 13.83 mg, 14.72 mol, 4% yield) as a white solid. UPLC-MS (ES+): m/z 910.3 [M+H]⁺. ¹H NMR (400 MHz, DMSO-d₆): δ 10.53 (s, 1H), 9.97 (bs, 1H), 8.38 (s, 1H), 7.76 (d, J=8.80 Hz, 1H), 7.63 (dd, J=2.80, 8.60 Hz, 1H), 7.36-7.27 (m, 4H), 7.11 (dd, J=2.40, 7.00 Hz, 1H), 5.03-4.85 (m, 1H), 3.94 (s, 3H), 3.89 (t, J=6.40 Hz, 2H), 3.13-2.89 (m, 8H), 2.74 (t, J=6.80 Hz, 2H), 2.64-2.56 (m, 4H), 2.47-2.38 (m, 2H), 2.30-2.06 (m, 6H), 2.00-1.82 (m, 2H), 1.80-1.70 (m, 4H), 1.03 (t, J=7.20 Hz, 3H).

### Synthesis D163: Synthesis of (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-fluoro-pyrrolidine-1-sulfonamide (Compound 267 and Compound 268)

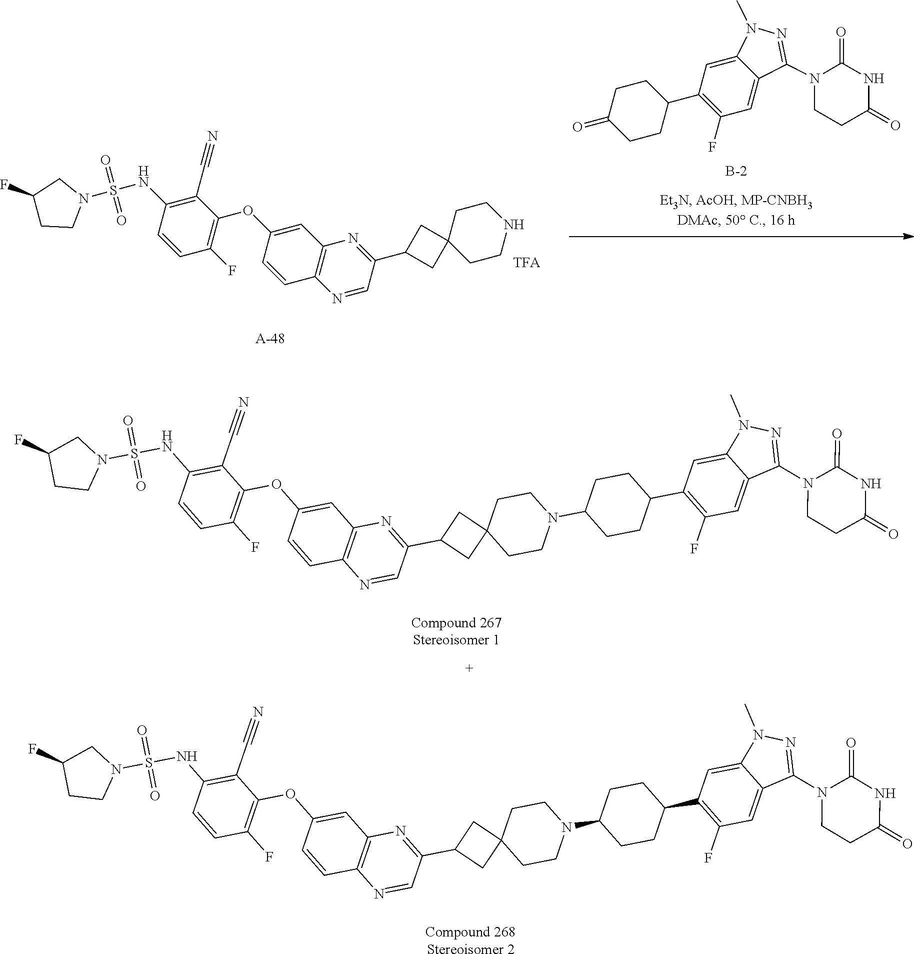

Into a 30 mL reaction vial containing a solution of (3R)-N-[3-[3-(7-azaspiro[3.5]nonan-2-yl)quinoxalin-6-yl]oxy-2-cyano-4-fluoro-phenyl]-3-fluoro-pyrrolidine-1-sulfonamide (A-48) (400.00 mg, 598.23 mol, TFA salt) in DMAc (5 mL) was added triethylamine (1.20 mmol, 166.76 L) and stirred for 15 min at room temperature. Subsequently, AcOH (1.79 mmol, 102.63 μL) and 1-[5-fluoro-1-methyl-6-(4-oxocyclohexyl)indazol-3-yl]hexahydropyrimidine-2,4-dione (B-2) (257.26 mg, 717.88 μmol) were added and stirred for 6 h at room temperature. Finally, MP-CNBH₃ (300 mg, 598.23 μmol) was added and stirred at 50° C. for 16 hours. After completion of the reaction, the reaction mixture was filtered through cotton and the filtrate was concentrated to afford 500 mg of mixture of cis and trans isomers. Further cis and trans isomers were separated by preparative HPLC [Column: X BRIDGE C18 (150 mm×21 mm×5 μm), Mobile phase A: 10 mM NH₄HCO₃ (pH:10 adjusted with ammonia solution) in milli-Q water; Mobile phase B: acetonitrile). The first eluting fraction (Peak-1) was collected and lyophilized to afford Compound 267 (assigned as stereoisomer 1).

Peak-1: (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-fluoro-pyrrolidine-1-sulfonamide (Compound 267, 36.58 mg, 39.93 mol, 7% yield) as an off-white solid. UPLC-MS (ES+): m/z 897.3 [M+H]⁺. ¹H NMR (400 MHz, DMSO-d₆): δ 10.56 (s, 1H), 8.89 (s, 1H), 8.77 (s, 1H), 8.12 (d, J=9.20 Hz, 1H), 7.69 (dd, J=2.80, 9.20 Hz, 1H), 7.54 (d, J=6.00 Hz, 1H), 7.40-7.31 (m, 3H), 7.08 (d, J=2.40 Hz, 1H), 5.40-5.20 (m, 1H), 4.00-3.89 (m, 7H), 3.52-3.42 (m, 2H), 3.30-3.25 (m, 2H), 3.20-3.10 (m, 2H), 3.08-2.85 (m, 3H), 2.76 (t, J=6.80 Hz, 2H), 2.35-2.05 (m, 7H), 2.05-1.90 (m, 4H), 1.90-1.60 (m, 7H).

Similarly, the second eluted fraction (Peak-2) was collected and lyophilized to afford Compound 268 (assigned as stereoisomer 2).

Peak-2: (3R)-N-[2-cyano-3-[3-[7-[4-[3-(2,4-dioxohexahydropyriimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-fluoro-pyrrolidine-1-sulfonamide (Compound 268, 46.35 mg, 51.25 mol, 9% yield) as an off-white solid. UPLC-MS (ES+): m/z 897.3 [M+H]⁺. ¹H NMR (400 MHz, DMSO-d₆): δ 10.56 (s, 1H), 8.77 (s, 1H), 8.52 (s, 1H), 8.12 (dd, J=4.00, 13.20 Hz, 1H), 7.68 (dd, J=2.80, 9.20 Hz, 1H), 7.50-7.34 (m, 4H), 7.09 (s, 1H), 5.40-5.20 (m, 1H), 4.20-3.70 (m, 7H), 3.60-3.40 (m, 3H), 3.25-3.10 (m, 3H), 3.10-2.85 (m, 2H), 2.76 (t, J=6.80 Hz, 2H), 2.48-2.40 (m, 1H), 2.30-2.20 (m, 3H), 2.18-1.70 (m, 15H).

### Synthesis D164: Synthesis of (3R)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-methoxy-pyrrolidine-1-sulfonamide (Compound 269)

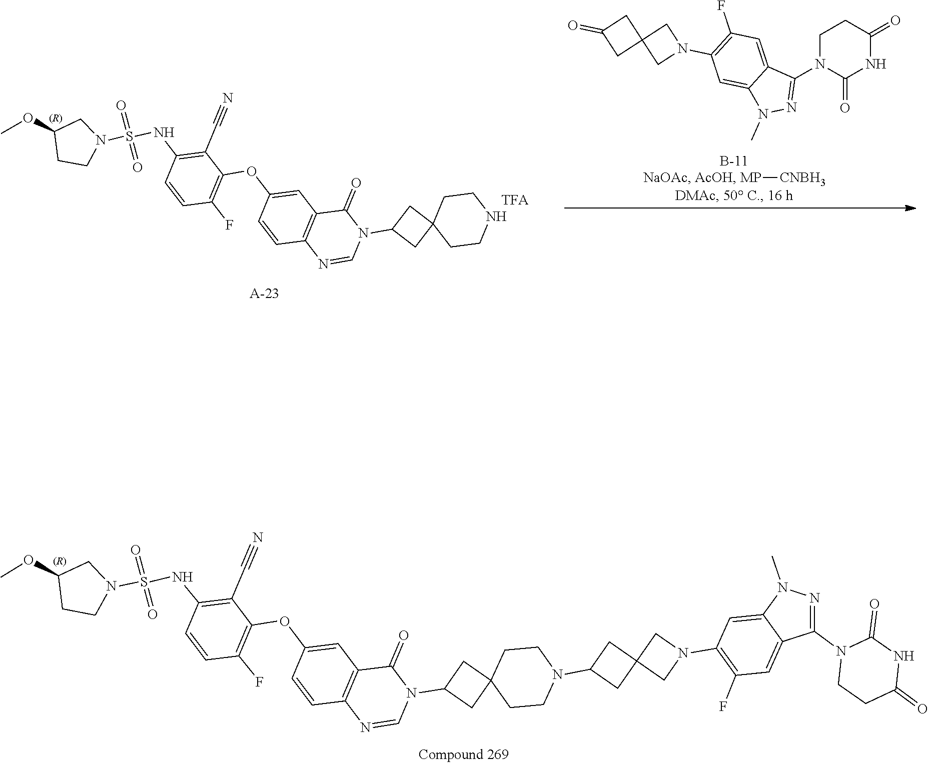

Into a 20 mL pressure vial containing a well stirred solution of (3R)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-23) (150 mg, 195.67 mol, TFA salt) and 1-[5-fluoro-1-methyl-6-(6-oxo-2-azaspiro[3.3]heptan-2-yl)indazol-3-yl]hexahydropyrimidine-2,4-dione (B-11) (99.54 mg, 195.67 mol) in DMAc (4 mL) were added AcOH (587.02 mol, 33.57 μL) and anhydrous sodium acetate (80.26 mg, 978.37 mol) at room temperature and stirred for 1 h at the same temperature. Subsequently, MP-CNBH₃ (200 mg, 195.67 mol) was added, and resulting reaction mixture was stirred at room temperature for 16 hours. After completion of the reaction, the reaction mixture was filtered through cotton pad and filtrate was concentrated under reduced pressure to get a crude residue which was purified by preparative HPLC [Column: X Bridge C18 (150 mm*19 mm*5 μm), Mobile phase A: 10 mM NH₄HCO₃ (pH was adjusted to 10 with NH₄OH) in milli-Q water; Mobile phase B: acetonitrile, flow rate:15 mL/min]. Fractions were collected and lyophilized to afford (3R)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-methoxy-pyrrolidine-1-sulfonamide (Compound 269, 28 mg, 29.70 mol, 15% yield) as an off-white solid. UPLC-MS (ES+): m/z 938.3 [M+H]⁺. ¹H NMR (400 MHz, DMSO-d₆): δ 10.51 (s, 1H), 9.21 (bs, 1H), 8.39 (s, 1H), 7.76 (d, J=9.20 Hz, 1H), 7.63 (dd, J=3.20, 9.00 Hz, 1H), 7.35-7.26 (m, 4H), 6.52 (d, J=7.60 Hz, 1H), 5.00-4.90 (m, 1H), 4.03 (s, 2H), 3.97-3.85 (m, 8H), 3.31-3.26 (m, 4H), 3.23-2.93 (m, 7H), 2.73 (t, J=6.80 Hz, 2H), 2.67-2.62 (m, 4H), 2.47-2.35 (m, 4H), 2.32-2.24 (m, 1H), 2.15-1.88 (m, 2H), 1.87-1.50 (m, 4H).

### Synthesis D165: Synthesis of N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]cyclopentanesulfonamide (Compound 270 and Compound 271)

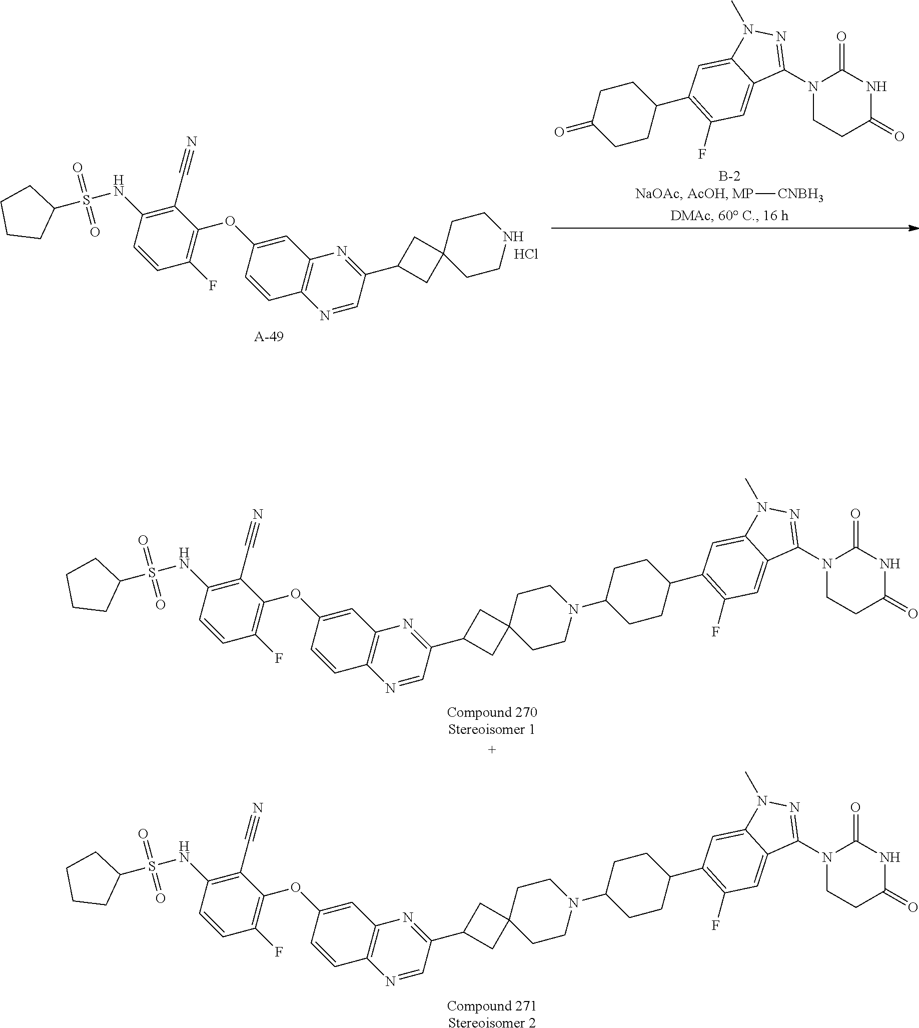

Into a 20 mL pressure glass vial containing a well stirred solution of N-[3-[3-(7-azaspiro[3.5]nonan-2-yl)quinoxalin-6-yl]oxy-2-cyano-4-fluoro-phenyl]cyclopentanesulfonamide (A-49) (0.350 g, 611.79 mol, HCl salt) and 1-[5-fluoro-1-methyl-6-(4-oxocyclohexyl)indazol-3-yl]hexahydropyrimidine-2,4-dione (B-2) (219.24 mg, 611.79 mol) in DMAc (5 mL) was added NaOAc (250.93 mg, 3.06 mmol) and the resulting mixture was stirred at room temperature for 1 hour. After 1 h, AcOH (1.84 mmol, 104.97 μL) was added and stirred at room temperature for 2 hours. Subsequently, MP-CNBH₃ (0.500 g, 611.79 mol) was added and stirred at 60° C. for 16 hours. After completion of the reaction, the reaction mixture was filtered through sintered funnel and filtrate was concentrated under reduced pressure to get crude, which was purified by reverse phase purification [Column: Redisep R_{f} Gold® C18-teledyne ISCO, 60 g), Mobile phase A: 10 mM NH₄HCO₃ in milli-Q water; Mobile phase B: acetonitrile]. The first eluting fraction (Peak-1) was lyophilized to afford Compound 270 (assigned as stereoisomer 1).

Peak-1: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]cyclopentanesulfonamide (Compound 270, 0.025 g, 27.79 mol, 5% yield) as an off-white solid. UPLC-MS (ES+): m/z 878.3[M+H]⁺. ¹H NMR (400 MHz, DMSO-d₆): δ 10.56 (s, 1H), 8.92 (bs, 1H), 8.77 (s, 1H), 8.12 (d, J=9.20 Hz, 1H), 7.68 (dd, J=2.80, 9.20 Hz, 1H), 7.54 (d, J=6.00 Hz, 1H), 7.40-7.33 (m, 3H), 7.11-7.09 (m, 1H), 4.02 (s, 3H), 3.91 (d, J=6.40 Hz, 2H), 3.50-3.38 (m, 1H), 3.18-3.05 (m, 2H), 2.93-2.85 (m, 2H), 2.76 (t, J=6.40 Hz, 3H), 2.30-2.24 (m, 2H), 2.21-2.12 (m, 2H), 2.05-1.60 (m, 19H), 1.55-1.45 (m, 2H).

Similarly, the second eluted fraction (Peak-2) was lyophilized to afford Compound 271 (assigned as stereoisomer 2).

Peak-2: N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]cyclopentanesulfonamide (Compound 271, 0.015 g, 16.95 mol, 3% yield) as an off-white solid. UPLC-MS (ES+): m/z 878.3 [M+H]⁺. ¹H NMR (400 MHz, DMSO-d₆): δ 10.56 (s, 1H), 8.77 (s, 1H), 8.55 (bs, 1H), 8.12 (d, J=8.80 Hz, 1H), 7.68 (dd, J=2.40, 9.20 Hz, 1H), 7.49-7.38 (m, 4H), 7.21-7.07 (m, 1H), 4.03 (s, 3H), 3.91 (d, J=6.40 Hz, 2H), 3.60-3.40 (m, 2H), 3.22-2.85 (m, 3H), 2.76 (t, J=6.40 Hz, 2H), 2.30-2.20 (m, 4H), 2.15-1.75 (m, 16H), 1.71-1.63 (m, 3H), 1.56-1.44 (m, 3H).

### Synthesis D166: Synthesis of (3S)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-methoxy-pyrrolidine-1-sulfonamide (Compound 272)

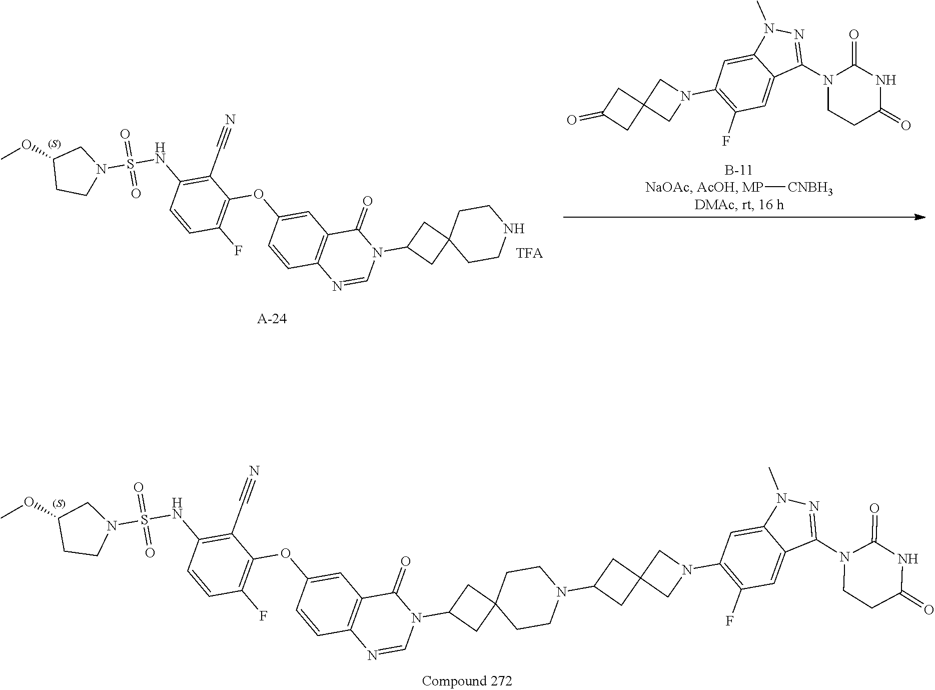

Into a 40 mL glass vial with pressure released cap containing a well-stirred solution of (3S)-N-[3-[3-(7-azaspiro[3.5]nonan-2-yl)-4-oxo-quinazolin-6-yl]oxy-2-cyano-4-fluoro-phenyl]-3-methoxy-pyrrolidine-1-sulfonamide (A-24) (200 mg, 252.63 mol, TFA salt) in DMAc (4 mL) was added NaOAc (103.62 mg, 1.26 mmol) and stirred for 10 min. Subsequently, AcOH (757.89 mol, 42.93 μL) and 1-[5-fluoro-1-methyl-6-(6-oxo-2-azaspiro[3.3]heptan-2-yl)indazol-3-yl]hexahydropyrimidine-2,4-dione (B-11) (132.31 mg, 277.89 mol) were added and stirred at room temperature for 1 hour. Finally, MP-CNBH₃ (200 mg, 252.63 mol) was added and stirred at room temperature for 16 hours. The reaction mass was diluted with dichloromethane (10 mL) and filtered, filtrate was concentrated under reduced pressure to afford crude residue which was purified by preparative HPLC [Column: X-Bridge C8 (250 mm*19 mm*5 μm), Mobile phase A: 10 mM NH₄HCO₃ & NH₄OH (pH_10) in milli-Q water; Mobile phase B: acetonitrile]. The fraction was lyophilized to afford (3S)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-methoxy-pyrrolidine-1-sulfonamide (Compound 272, 40.0 mg, 42.29 mol, 17% yield) as an off-white solid. UPLC-MS (ES+): m/z 938.3 [M+H]⁺. ¹H NMR (400 MHz, DMSO-d₆): δ 10.51 (s, 1H), 9.52 (bs, 1H), 8.39 (s, 1H), 7.76 (d, J=8.80 Hz, 1H), 7.63 (dd, J=2.80, 8.80 Hz, 1H), 7.52-7.45 (m, 1H), 7.38-7.32 (m, 2H), 7.27 (d, J=12.80 Hz, 1H), 6.52 (d, J=7.60 Hz, 1H), 5.02-4.90 (m, 1H), 4.03 (s, 2H), 3.94-3.91 (m, 3H), 3.90-3.85 (m, 5H), 3.30-3.26 (m, 2H), 3.18 (s, 3H), 3.16-3.02 (m, 4H), 2.95-2.79 (m, 3H), 2.73 (t, J=6.80 Hz, 2H), 2.47-2.25 (m, 7H), 2.06-1.55 (m, 7H).

### Synthesis D167: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]propane-2-sulfonamide (Compound 273)

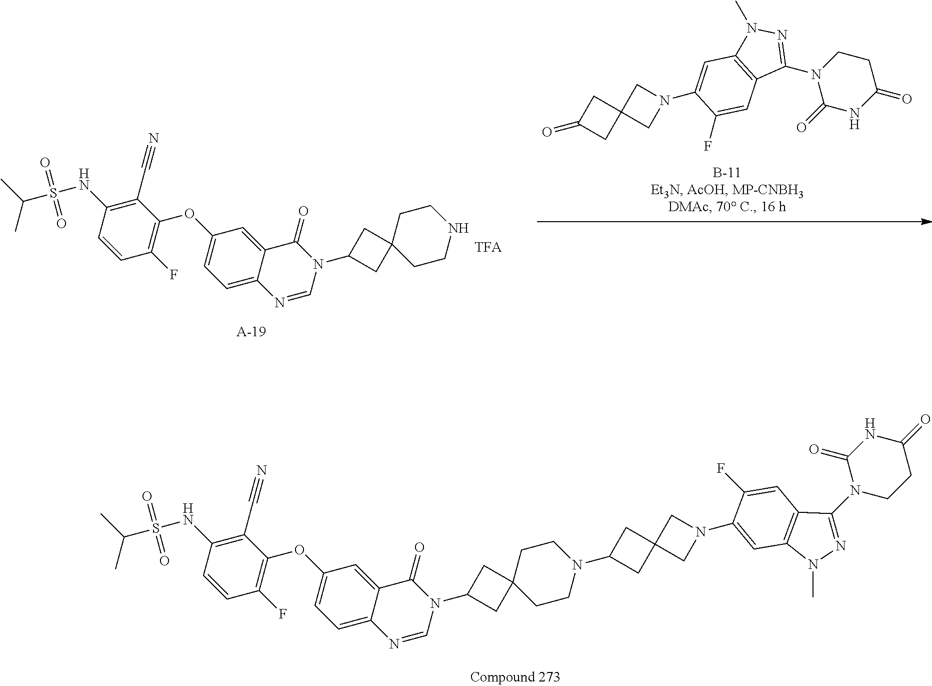

Into a 15 mL pressure vial containing a well-stirred solution of N-[3-[3-(7-azaspiro[3.5]nonan-2-yl)-4-oxo-quinazolin-6-yl]oxy-2-cyano-4-fluoro-phenyl]propane-2-sulfonamide (A-19) (200 mg, 278.29 mol, TFA salt) in DMAc (3 mL) was added triethylamine (1.39 mmol, 193.94 μL) and resulting mixture was stirred for 1 h at room temperature. Subsequently, 1-[5-fluoro-1-methyl-6-(6-oxo-2-azaspiro[3.3]heptan-2-yl)indazol-3-yl]hexahydropyrimidine-2,4-dione (B-11) (120.17 mg, 278.29 mol) and AcOH (834.87 mol, 47.75 μL) were added and stirred for 5 hours. Finally, MP-CNBH₃ (200 mg) was added and resulting reaction mixture was stirred at 70° C. for 16 hours. After completion of the reaction, the reaction mixture was filtered through sintered funnel and washed with dichloromethane. Filtrate was concentrated under reduced pressure to get a crude residue which was purified by preparative HPLC [Column: X-Bridge C18 (150 mm*19 mm*5 μm), Mobile Phase A: 10 mM NH₄HCO₃ in milli-Q water; Mobile phase B: acetonitrile]. Fractions were collected and lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]propane-2-sulfonamide (Compound 273, 49 mg, 55.05 mol, 20% yield) as an off-white solid. UPLC-MS (ES+): m/z 881.3 [M+H]⁺. ¹H NMR (400 MHz, DMSO-d₆): δ 10.49 (s, 1H), 8.35 (s, 1H), 7.76 (d, J=8.80 Hz, 1H), 7.62 (dd, J=2.80, 8.80 Hz, 1H), 7.44-7.36 (m, 1H), 7.35-7.28 (m, 2H), 7.27-7.22 (m, 1H), 6.51 (d, J=7.20 Hz, 1H), 4.98-4.88 (m, 1H), 4.01 (s, 2H), 3.92-3.85 (m, 8H), 3.64-3.62 (m, 2H), 3.15-2.69 (m, 8H), 2.40-2.26 (m, 6H), 1.88-1.71 (m, 3H), 1.22 (d, J=6.80 Hz, 6H).

### Synthesis D168: Synthesis of 6-[2-cyano-3-[[ethyl(methyl)sulfamoyl]amino]-6-fluoro-phenoxy]-3-[1-[7-[3-(2,4-dioxohexahydropyrimidin-1-yl)-5-fluoro-1-methyl-indazol-6-yl]-7-azaspiro[3.5]nonan-2-yl]-4-piperidyl]-4-oxo-quinazoline (Compound 274)

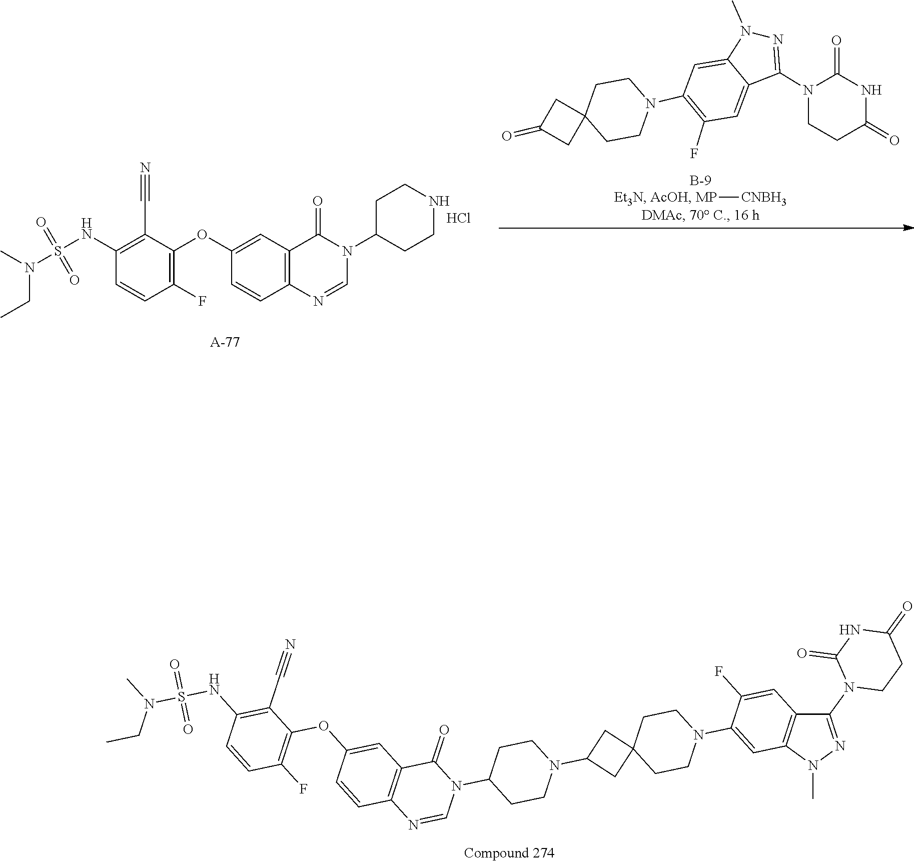

Into a 20 mL pressure vial containing a well stirred solution of 6-[2-cyano-3-[[ethyl(methyl)sulfamoyl]amino]-6-fluoro-phenoxy]-4-oxo-3-(4-piperidyl)quinazoline (A-77) (1.1 g, 1.80 mmol, 71.94% yield, HCl salt) in DMAc (3 mL) was added triethylamine (896.44 mol, 124.95 μL) and stirred for 30 min at room temperature. Subsequently, AcOH (896.44 mol, 51.26 μL) and 1-[5-fluoro-1-methyl-6-(2-oxo-7-azaspiro[3.5]nonan-7-yl)indazol-3-yl]hexahydropyrimidine-2,4-dione (B-9) (120 mg, 298.81 μmol) were added and stirred for 3 h at room temperature. Finally, MP-CNBH₃ (300 mg, 298.81 mol) was added and stirred at 70° C. for 16 hours. After completion of the reaction, the reaction mixture was filtered through cotton pad and filtrate was concentrated under reduced pressure to afford crude residue, which was purified by preparative HPLC [Column: X-Bridge C18 (250 mm*19 mm*5 μm), Mobile phase A: 10 mM NH₄HCO₃ in water and NH₄OH (pH-10); Mobile phase B: acetonitrile, Flow rate: 14 mL/min]. Fractions were concentrated under reduced pressure at 40° C. and lyophilized to afford 6-[2-cyano-3-[[ethyl(methyl)sulfamoyl]amino]-6-fluoro-phenoxy]-3-[1-[7-[3-(2,4-dioxohexahydropyrimidin-1-yl)-5-fluoro-1-methyl-indazol-6-yl]-7-azaspiro[3.5]nonan-2-yl]-4-piperidyl]-4-oxo-quinazoline (Compound 274, 95.18 mg, 104.93 mol, 35% yield) as an off-white solid. UPLC-MS (ES+): m/z 884.20 [M+H]⁺. ¹H NMR (400 MHz, DMSO-d₆): δ 10.52 (s, 1H), 9.90 (bs, 1H), 8.39 (s, 1H), 7.77 (d, J=8.80 Hz, 1H), 7.67-7.55 (m, 2H), 7.38-7.32 (m, 3H), 7.11 (d, J=7.20 Hz, 1H), 5.72-4.58 (m, 1H), 3.94 (s, 3H), 3.89 (t, J=6.80 Hz, 2H), 3.25-2.94 (m, 9H), 2.75-2.64 (m, 5H), 2.30-2.10 (m, 5H), 2.03-1.90 (m, 3H), 2.85-2.67 (m, 6H), 1.04 (t, J=7.20 Hz, 3H).

### Synthesis D169: Synthesis of (3S)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-methoxy-pyrrolidine-1-sulfonamide (Compound 275 and Compound 276)

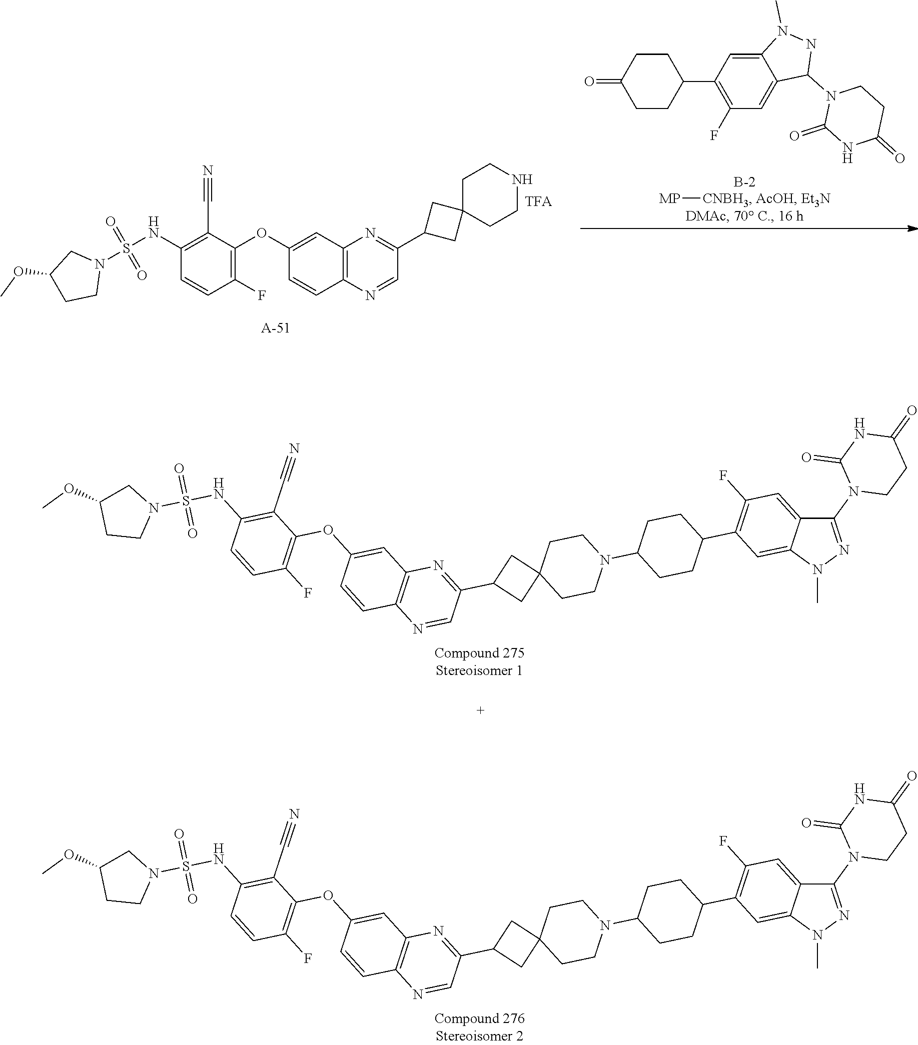

Into a 20 mL pressure vial containing a well-stirred solution of (3S)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-51) (264.37 mg, 337.90 mol, TFA salt) in anhydrous DMAc (6 mL) was added triethylamine (1.69 mmol, 235.48 μL) at room temperature and stirred for 30 min. To the above mixture, 1-[5-fluoro-1-methyl-6-(4-oxocyclohexyl)indazol-3-yl]hexahydropyrimidine-2,4-dione (B-2) (121.09 mg, 337.90 mol) and AcOH (1.01 mmol, 58.53 μL) were added at room temperature and stirred for 5 hours. Finally, MP-CNBH₃ (265 mg, 337.90 mol) was added and resulting reaction mixture was stirred at 70° C. for 16 hours. After completion of the reaction, the reaction mixture was filtered through sintered funnel, washed with dichloromethane and filtrate was concentrated under reduced pressure to get a crude residue, which was further purified by preparative HPLC [Column: X-SELECT CSH (150 mm*19 mm*5 μm), Mobile phase A: 10 mM NH₄HCO₃ in milli-Q water; Mobile phase B: acetonitrile]. The first eluting fraction (Peak-1) was lyophilized to afford Compound 275 (assigned as stereoisomer 1).

Peak-1: (3S)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-methoxy-pyrrolidine-1-sulfonamide (Compound 275, 35.37 mg, 38.69 mol, 11% yield) as an off-white solid. UPLC-MS: m/z 909.1 [M+H]⁺. ¹H NMR (400 MHz, DMSO-d₆): δ 10.56 (s, 1H), 8.91 (bs, 1H), 8.80 (s, 1H), 8.12 (d, J=8.80 Hz, 1H), 7.69 (dd, J=2.80, 8.80 Hz, 1H), 7.54 (d, J=6.00 Hz, 1H), 7.45-7.26 (m, 3H), 7.08-7.03 (m, 1H), 4.00 (s, 3H), 3.97-3.89 (m, 4H), 3.55-3.42 (m, 1H), 3.29-3.26 (m, 1H), 3.18-2.87 (m, 10H), 2.76 (t, J=6.80 Hz, 2H), 2.48-2.35 (m, 1H), 2.31-2.11 (m, 6H), 2.07-1.59 (m, 12H).

Similarly, the second eluted fraction (Peak-2) was lyophilized to afford Compound 276 (assigned as stereoisomer 2).

Peak-2: (3S)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-methoxy-pyrrolidine-1-sulfonamide (Compound 276, 36.73 mg, 40.06 mol, 12% yield) as an off-white solid. UPLC-MS: m/z 909.2 [M+H]⁺. ¹H NMR (400 MHz, DMSO-d₆): δ 10.56 (s, 1H), 8.77 (s, 1H), 8.54 (bs, 1H), 8.12 (d, J=9.20 Hz, 1H), 7.68 (dd, J=2.80, 9.00 Hz, 1H), 7.55-7.30 (m, 4H), 7.10 (s, 1H), 4.03 (s, 3H), 3.99-3.89 (m, 4H), 3.59-3.42 (m, 2H), 3.28-3.25 (m, 1H), 3.21-2.89 (m, 9H), 2.76 (t, J=6.80 Hz, 2H), 2.47-2.35 (m, 1H), 2.32-2.20 (m, 3H), 2.19-1.50 (m, 15H).

### Synthesis D170: Synthesis of (1R,5S)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-azabicyclo[3.1.0]hexane-3-sulfonamide (Compound 277)

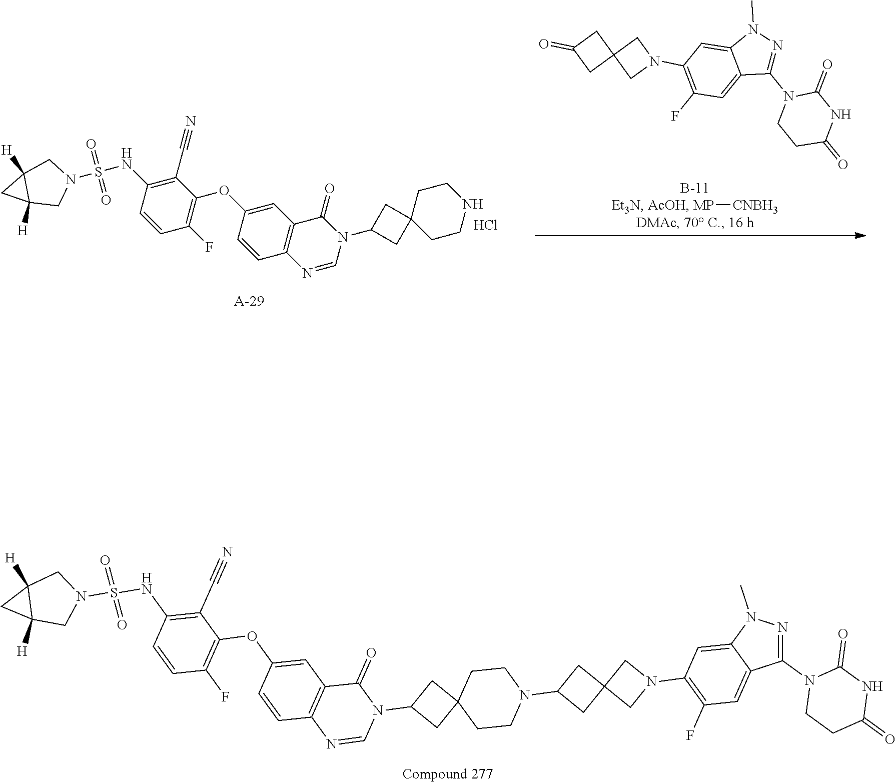

Into a 30 mL glass vial containing a well stirred solution of (1R,5S)-N-[3-[3-(7-azaspiro[3.5]nonan-2-yl)-4-oxo-quinazolin-6-yl]oxy-2-cyano-4-fluoro-phenyl]-3-azabicyclo[3.1.0]hexane-3-sulfonamide (A-29) (200 mg, 280.82 mol, HCl salt) in DMAc (5 mL) was added triethylamine (842.47 mol, 117.42 μL) at 5° C. and stirred for 30 min. Subsequently, 1-[5-fluoro-1-methyl-6-(6-oxo-2-azaspiro[3.3]heptan-2-yl)indazol-3-yl]hexahydropyrimidine-2,4-dione (B-11) (157.15 mg, 308.91 mol) and AcOH (561.65 mol, 32.12 μL) were added at room temperature and the resulting mixture was stirred at room temperature for 3 hours. Finally, MP-CNBH₃ (250 mg, 280.82 mol) was added, and the mixture was stirred at 70° C. for 16 hours. After completion of the reaction, the reaction mixture was filtered and concentrated under reduced pressure to get a crude compound, which was purified by prep HPLC [Column: X-Bridge C18 (250 mm*19 mm*5 m), mobile phase A: 10 mM NH₄HCO₃ in milli-Q water; mobile phase B: acetonitrile]. The pure fractions were lyophilized to afford (1R,5S)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-azabicyclo[3.1.0]hexane-3-sulfonamide (Compound 277, 42 mg, 44.61 mol, 16% yield) as an off-white solid. UPLC-MS (ESI+): m/z 920.3 [M+H]⁺. ¹H NMR (400 MHz, DMSO-d₆): δ 10.51 (s, 1H), 9.33 (bs, 1H), 8.39 (s, 1H), 7.76 (d, J=9.20 Hz, 1H), 7.63 (dd, J=3.20, 9.00 Hz, 1H), 7.50-7.41 (m, 1H), 7.34 (d, J=3.20 Hz, 1H), 7.30-7.22 (m, 2H), 6.52 (d, J=7.60 Hz, 1H), 5.00-4.90 (m, 1H), 4.03 (s, 2H), 3.94 (s, 2H), 3.90-3.82 (m, 6H), 3.20-3.09 (m, 5H), 2.73 (t, J=6.80 Hz, 3H), 2.40-2.25 (m, 4H), 2.00-1.61 (m, 4H), 1.51-1.42 (m, 2H), 0.52-0.45 (m, 1H), 0.39-0.28 (m, 1H).

### Synthesis D171: Synthesis of (3R)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]-3-methoxy-pyrrolidine-1-sulfonamide (Compound 278)

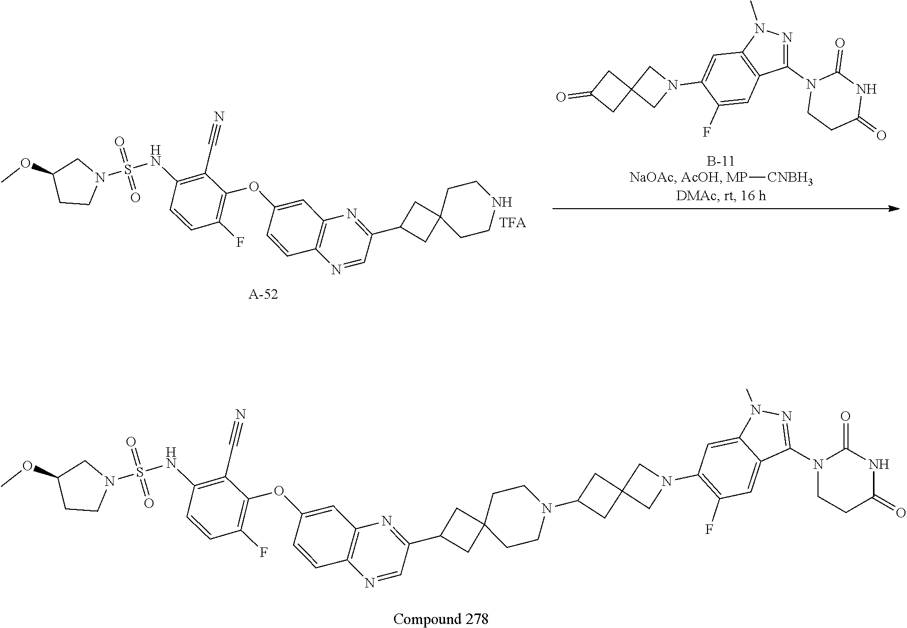

Into a 20 mL glass vial with pressure released cap containing a well-stirred solution of (3R)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-52) (180 mg, 245.93 mol, TFA salt) in DMAc (5 mL) was added NaOAc (100.87 mg, 1.23 mmol) at room temperature and stirred for 10 min. Subsequently, AcOH (737.80 mol, 42.2 μL) and 1-[5-fluoro-1-methyl-6-(6-oxo-2-azaspiro[3.3]heptan-2-yl)indazol-3-yl]hexahydropyrimidine-2,4-dione (B-11) (125.11 mg, 245.93 mol,) were added at room temperature and stirred for 2 hours. Finally, MP-CNBH₃ (180 mg, 245.93 mol) was added and stirred at room temperature for 16 hours. After completion of the reaction, the reaction mixture was filtered through cotton pad, filtrate was concentrated under reduced pressure to get crude residue, which was purified by prep HPLC [Column: X-Bridge C18 (150 mm*19 mm*5 μm) and [Mobile phase A:10 mM NH₄HCO₃ & NH₄OH (pH-10) in milli-Q water; Mobile phase B: acetonitrile]. Pure fractions were lyophilized to afford (3R)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]-3-methoxy-pyrrolidine-1-sulfonamide (Compound 278, 14 mg, 15.05 mol, 6% yield) as an off-white solid. UPLC-MS (ES+): m/z 922.3. [M+H]⁺. ¹H NMR (400 MHz, DMSO-d₆): δ 10.51 (s, 1H), 9.34 (bs, 1H), 8.78 (s, 1H), 8.13 (d, J=9.20 Hz, 1H), 7.69 (dd, J=2.80, 9.20 Hz, 1H), 7.50-7.12 (m, 3H), 7.19 (s, 1H), 6.53 (d, J=7.60 Hz, 1H), 4.03-3.79 (m, 12H), 3.38-3.01 (m, 8H), 2.75-2.68 (m, 3H), 2.34-2.24 (m, 7H), 1.99-1.72 (m, 6H).

### Synthesis D172: Synthesis of (3S)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]-3-methoxy-pyrrolidine-1-sulfonamide (Compound 279)

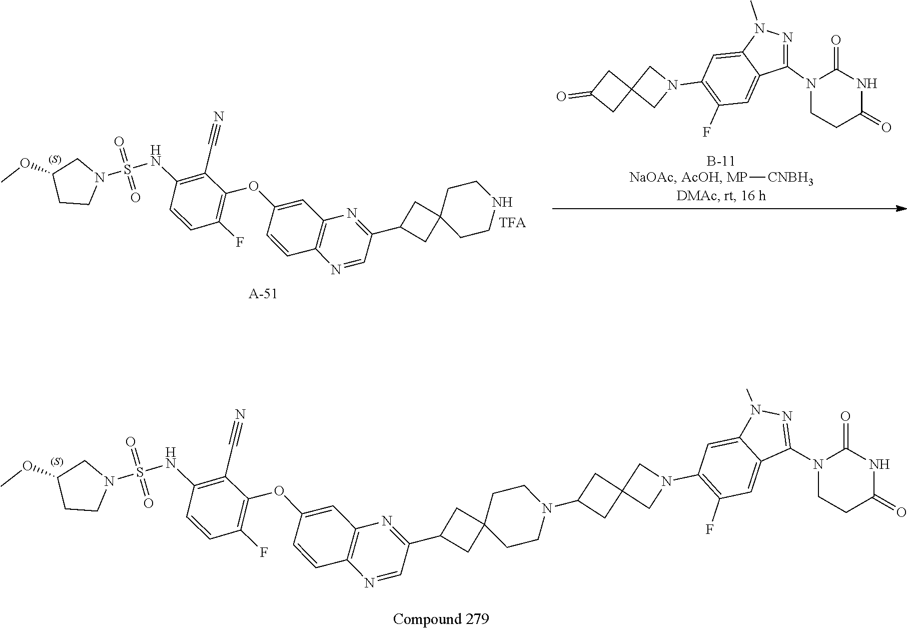

Into a 20 mL pressure vial containing a well-stirred solution of (3S)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-51) (150 mg, 191.72 mol, TFA salt) and 1-[5-fluoro-1-methyl-6-(6-oxo-2-azaspiro[3.3]heptan-2-yl)indazol-3-yl]hexahydropyrimidine-2,4-dione (B-11) (71.20 mg, 191.72 mol) in DMAc (3 mL) were added NaOAc (78.64 mg, 958.62 mol) and AcOH (575.17 mol, 32.89 μL) at room temperature and stirred for 1 hour. Subsequently, MP-CNBH₃ (150 mg, 191.72 mol) was added and was stirred for 16 hours. After completion of the reaction, the reaction mixture was diluted with dichloromethane (10 mL), filtered through cotton pad and filtrate was concentrated under reduced pressure to get crude residue which was purified by preparative HPLC [Column: X-Bridge C18 (150 mm*19 mm*5 m), Mobile phase A: 10 mM NH₄HCO₃ in milli-Q water; Mobile phase B: acetonitrile, flow rate: 15 mL/min]. Fractions were collected and lyophilized to afford (3S)-N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]-3-methoxy-pyrrolidine-1-sulfonamide (Compound 279, 21 mg, 22.13 mol, 12% yield) as an off-white solid. UPLC-MS (ES+): m/z 922.2 [M+H]⁺. ¹H NMR (400 MHz, DMSO-d₆): δ 10.51 (s, 1H), 9.34 (bs, 1H), 8.78 (s, 1H), 8.12 (d, J=9.20 Hz, 1H), 7.68 (dd, J=2.80, 9.00 Hz, 1H), 7.46-7.37 (m, 2H), 7.29-7.26 (m, 1H), 7.15-7.09 (m, 1H), 6.53 (d, J=7.20 Hz, 1H), 4.03 (s, 2H), 3.94-3.86 (m, 10H), 3.29-3.27 (m, 1H), 3.18-3.12 (m, 4H), 3.10-3.01 (m, 4H), 2.73 (t, J=6.80 Hz, 3H), 2.41-2.12 (m, 8H), 2.01-1.92 (m, 2H), 1.79-1.58 (m, 5H).

### Synthesis D173: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-methoxy-azetidine-1-sulfonamide (Compound 280)

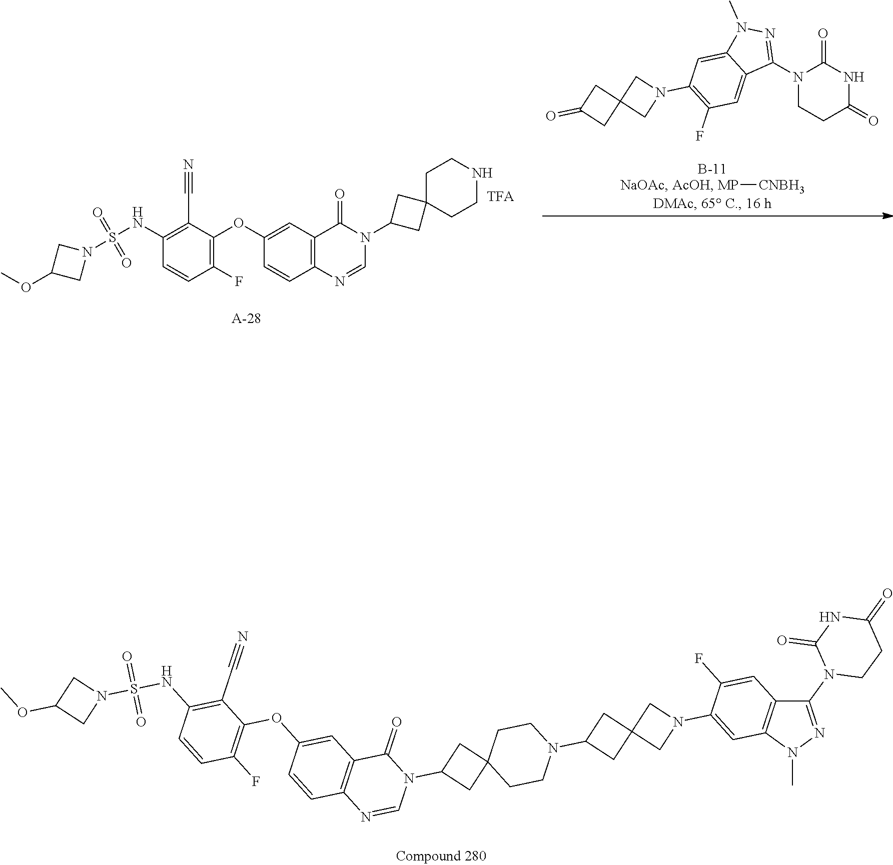

Into a 20 mL pressure vial containing a well stirred solution of N-[3-[3-(7-azaspiro[3.5]nonan-2-yl)-4-oxo-quinazolin-6-yl]oxy-2-cyano-4-fluoro-phenyl]-3-methoxy-azetidine-1-sulfonamide (A-28) (0.25 g, 306.39 mol, TFA salt) in DMAc (3 mL) was added NaOAc (125.67 mg, 1.53 mmol) and stirred for 30 min at room temperature. Subsequently, AcOH (919.17 mol, 52.67 μL) and 1-[5-fluoro-1-methyl-6-(6-oxo-2-azaspiro[3.3]heptan-2-yl)indazol-3-yl]hexahydropyrimidine-2,4-dione (B-11) (197.47 mg, 459.59 mol) were added and resulting mixture was stirred for 1 h at room temperature. Finally, MP-CNBH₃ (100 mg, 306.39 mol) was added and stirred at 65° C. for 16 hours. After completion of the reaction, the reaction mixture was filtered through cotton pad and filtrate was concentrated under reduced pressure to afford crude residue, which was purified by preparative HPLC [Column: X-Bridge C18 (150 mm*19 mm*5 m), Mobile phase A: 10 mM NH₄HCO₃ and NH₄OH (pH-10); Mobile phase B: acetonitrile, Flow rate: 14 mL/min]. Fractions were concentrated under reduced pressure at 40° C. and lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-4-oxo-quinazolin-6-yl]oxy-4-fluoro-phenyl]-3-methoxy-azetidine-1-sulfonamide (Compound 280, 60 mg, 64.33 mol, 21% yield) as a white solid. UPLC-MS (ES+): m/z 924.2 [M+H]⁺. ¹H NMR (400 MHz, DMSO-d₆): δ 10.51 (s, 1H), 9.23 (bs, 1H) 8.38 (s, 1H), 7.76 (d, J=8.80 Hz, 1H), 7.62 (dd, J=3.20, 8.80 Hz, 1H), 7.39-7.26 (m, 4H), 6.53 (d, J=7.20 Hz, 1H), 4.98-4.94 (m, 1H), 4.04-4.01 (m, 3H), 3.95 (s, 2H), 3.90-3.86 (m, 2H), 3.72-3.65 (m, 3H), 3.56-3.48 (m, 2H), 3.37-3.33 (m, 2H), 3.16 (s, 3H), 2.73 (t, J=6.80 Hz, 3H), 2.61-2.55 (m, 2H), 2.45-2.26 (m, 8H), 2.11-1.65 (m, 5H).

### Synthesis D174: Synthesis of 7-[2-cyano-3-[[ethyl(methyl)sulfamoyl]amino]-6-fluoro-phenoxy]-2-[[2-[4-[3-(2,4-dioxohexahydropyrimidin-1-yl)-5-fluoro-1-methyl-indazol-6-yl]cyclohexyl]-2-azaspiro[3.3]heptan-6-yl]oxy]quinoxaline (Compound 281 and Compound 282)

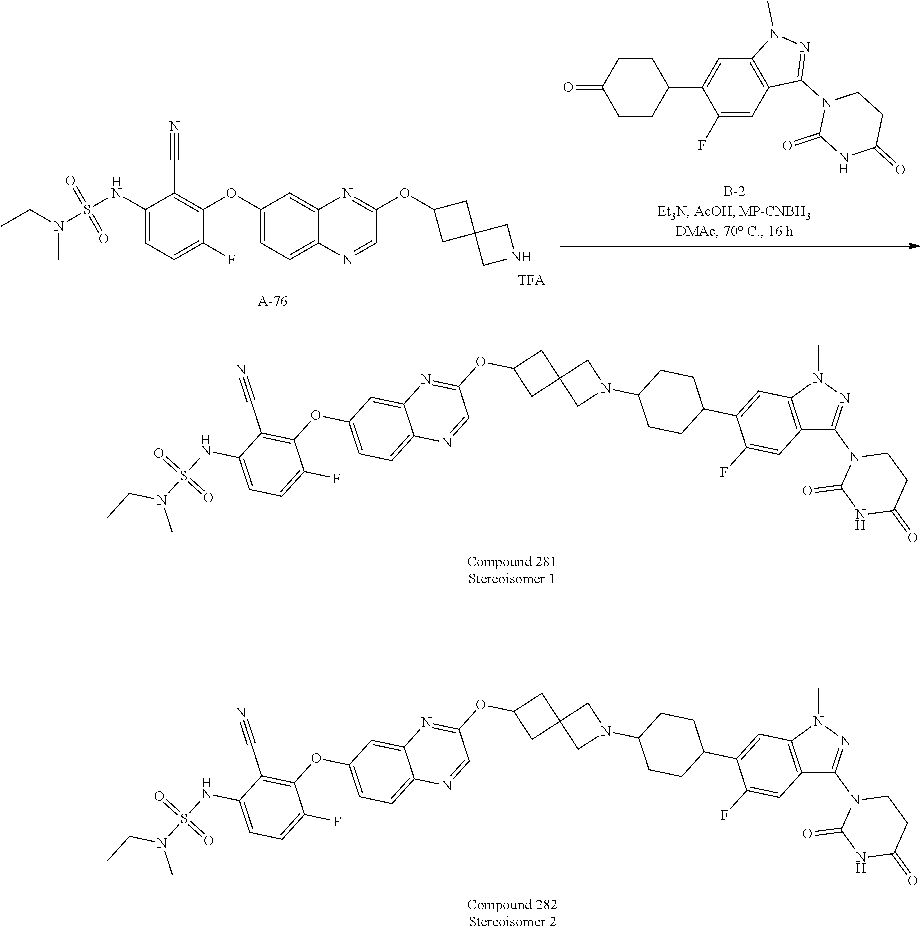 [SMILES: CCN(C)S(=O)(=O)Nc1ccc(F)c(Oc2ccc3ncc(OC4CC5(CNC5)C4)nc3c2)c1C#N ; Cn1nc(N2CCC(=O)NC2=O)c2cc(F)c(C3CCC(=O)CC3)cc21 ; "A-76" ; CCN(C)S(=O)(=O)Nc1ccc(F)c(Oc2ccc3ncc(OC4CC5(C4)CN(C4CCC(c6cc7c(cc6F)c(N6CCC(=O)NC6=O)nn7C)CC4)C5)nc3c2)c1C#N ; CCN(C)S(=O)(=O)Nc1ccc(F)c(Oc2ccc3ncc(OC4CC5(C4)CN(C4CCC(c6cc7c(cc6F)c(N6CCC(=O)NC6=O)nn7C)CC4)C5)nc3c2)c1C#N]

Into a 20 mL pressure vial containing a well-stirred solution of 2-(2-azaspiro[3.3]heptan-6-yloxy)-7-[2-cyano-3-[[ethyl(methyl)sulfamoyl]amino]-6-fluoro-phenoxy]quinoxaline (A-76) (298.10 mg, 450.06 mol, TFA salt) in anhydrous DMAc (8 mL) was added triethylamine (2.25 mmol, 313.65 μL) at room temperature and mixture was stirred for 30 min. To the above mixture, 1-[5-fluoro-1-methyl-6-(4-oxocyclohexyl)indazol-3-yl]hexahydropyrimidine-2,4-dione (B-2) (162.92 mg, 450.06 mol) and AcOH (1.35 mmol, 77.21 μL) were added at room temperature and stirred for 5 hours. Finally, MP-CNBH₃ (300 mg) was added and resulting reaction mixture was stirred at 70° C. for 16 hours. After completion of the reaction, the reaction mixture was filtered through sintered funnel, washed with dichloromethane and the filtrate was concentrated under reduced pressure to get a crude residue, which was further purified by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction (Peak-1) was lyophilized to afford Compound 281 (assigned as stereoisomer 1).

Peak-1: 7-[2-cyano-3-[[ethyl(methyl)sulfamoyl]amino]-6-fluoro-phenoxy]-2-[[2-[4-[3-(2,4-dioxohexahydropyrimidin-1-yl)-5-fluoro-1-methyl-indazol-6-yl]cyclohexyl]-2-azaspiro[3.3]heptan-6-yl]oxy]quinoxaline (Compound 281, 20.07 mg, 23.18 mol, 5% yield) as an off-white solid. UPLC-MS (ES+): m/z 855.2 [M+H]⁺. ¹H NMR (400 MHz, DMSO-d₆): δ 10.56 (s, 1H), 9.72 (bs, 1H), 8.47 (s, 1H), 8.05 (d, J=8.8 MHz, 1H), 7.53-7.52 (m, 2H), 7.40-7.37 (m, 2H), 7.32-7.18 (m, 1H), 6.98-6.78 (m, 1H), 5.24-5.20 (m, 1H), 4.47-4.09 (m, 3H), 3.99 (s, 3H), 3.90 (t, J=6.8 Hz, 2H), 3.12-2.94 (m, 3H), 2.93-2.82 (m, 2H), 2.75 (t, J=6.4 Hz, 3H), 2.62-2.58 (m, 3H), 2.48-2.37 (m, 3H), 2.15-1.90 (m, 4H), 1.68-1.52 (m, 2H), 1.41-1.20 (m, 2H), 1.04 (t, J=7.2 Hz, 3H).

Similarly, the second eluted fraction (Peak-2) was lyophilized to afford Compound 282 (assigned as stereoisomer 2).

Peak-2: 7-[2-cyano-3-[[ethyl(methyl)sulfamoyl]amino]-6-fluoro-phenoxy]-2-[[2-[4-[3-(2,4-dioxohexahydropyrimidin-1-yl)-5-fluoro-1-methyl-indazol-6-yl]cyclohexyl]-2-azaspiro[3.3]heptan-6-yl]oxy]quinoxaline (Compound 282, 23.41 mg, 26.57 mol, 6% yield) as an off-white solid. UPLC-MS (ES+): m/z 855.2 [M+H]⁺. ¹H NMR (400 MHz, DMSO-d₆): δ 10.56 (s, 1H), 9.44 (bs, 1H), 8.47 (s, 1H), 8.05 (d, J=9.2 MHz, 1H), 7.70-7.10 (m, 5H), 7.02-6.79 (m, 1H), 5.24-5.22 (m, 1H), 4.49-4.10 (m, 3H), 4.02 (s, 3H), 3.91 (t, J=6.8 Hz, 2H), 3.58-3.52 (m, 1H), 3.08-2.91 (m, 4H), 2.75 (t, J=6.8 Hz, 3H), 2.62-2.55 (m, 3H), 2.45-2.37 (m, 3H), 1.92-1.51 (m, 8H), 1.03 (t, J=7.2 Hz, 3H)

### Synthesis D175: Synthesis of N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]cyclopentanesulfonamide (Compound 283)

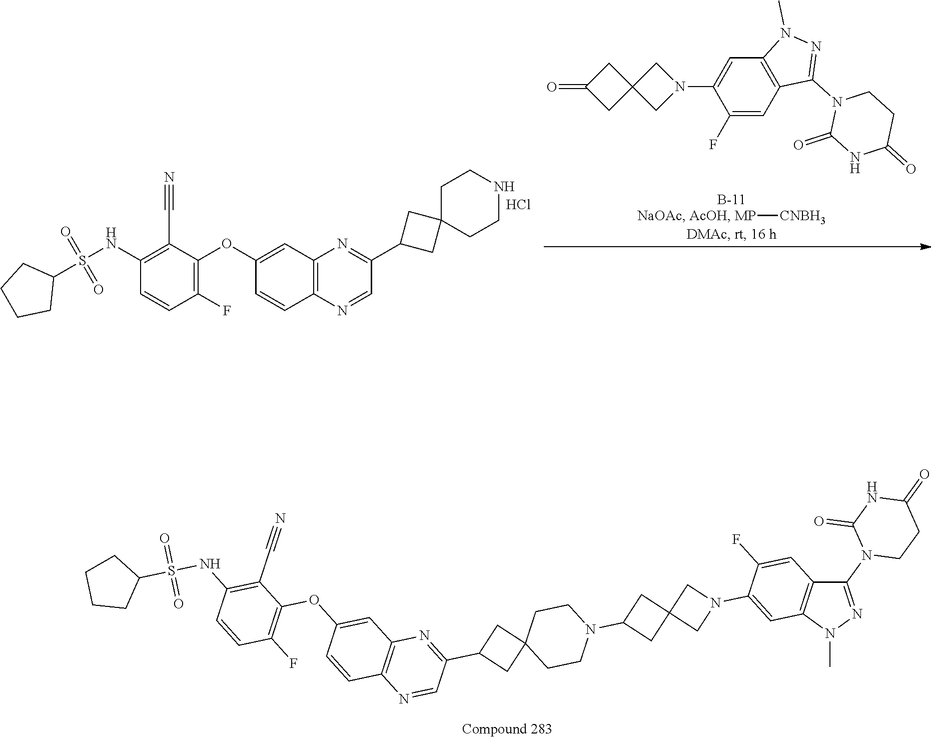

Into a 20 mL pressure vial containing a well-stirred solution of N-[3-[3-(7-azaspiro[3.5]nonan-2-yl)quinoxalin-6-yl]oxy-2-cyano-4-fluoro-phenyl]cyclopentanesulfonamide (A-49) (170 mg, 264.47 mol, HCl salt) in anhydrous DMAc (3 mL) was added NaOAc (108.47 mg, 1.32 mmol) at room temperature and resulting mixture was stirred for 30 min. To the above mixture, 1-[5-fluoro-1-methyl-6-(6-oxo-2-azaspiro[3.3]heptan-2-yl)indazol-3-yl]hexahydropyrimidine-2,4-dione (B-11) (163.69 mg, 317.36 mol) and AcOH (793.40 mol, 45.38 μL) were added at room temperature and stirred for 1 hour. Finally, MP-CNBH₃ (170 mg) was added and stirred at room temperature for 16 hours. After completion of the reaction, the reaction mixture was filtered through sintered funnel, washed with dichloromethane and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water; Mobile phase B: acetonitrile]. Fractions were collected and lyophilized to afford N-[2-cyano-3-[3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]quinoxalin-6-yl]oxy-4-fluoro-phenyl]cyclopentanesulfonamide (Compound 283.29 mg, 32.09 mol, 12% yield) as an off-white solid. UPLC-MS (ES+): m/z 891.2 [M+H]⁺. ¹H NMR (400 MHz, DMSO-d₆): δ 10.51 (s, 1H), 9.44 (bs, 1H), 8.78 (s, 1H), 8.12 (d, J=9.20 Hz, 1H), 7.69 (dd, J=2.80, 9.20 Hz, 1H), 7.52-7.36 (m, 2H), 7.27 (d, J=12.80 Hz, 1H), 7.14 (s, 1H), 6.52 (d, J=7.60 Hz, 1H), 4.03 (s, 2H), 3.93-3.86 (m, 8H), 3.65-3.40 (m, 3H), 3.15-2.72 (m, 5H), 2.34-2.19 (m, 6H), 1.98-1.80 (m, 6H), 1.75-1.47 (m, 6H).

### Synthesis D176: Synthesis of (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-methoxy-pyrrolidine-1-sulfonamide (Compound 284 and Compound 285)

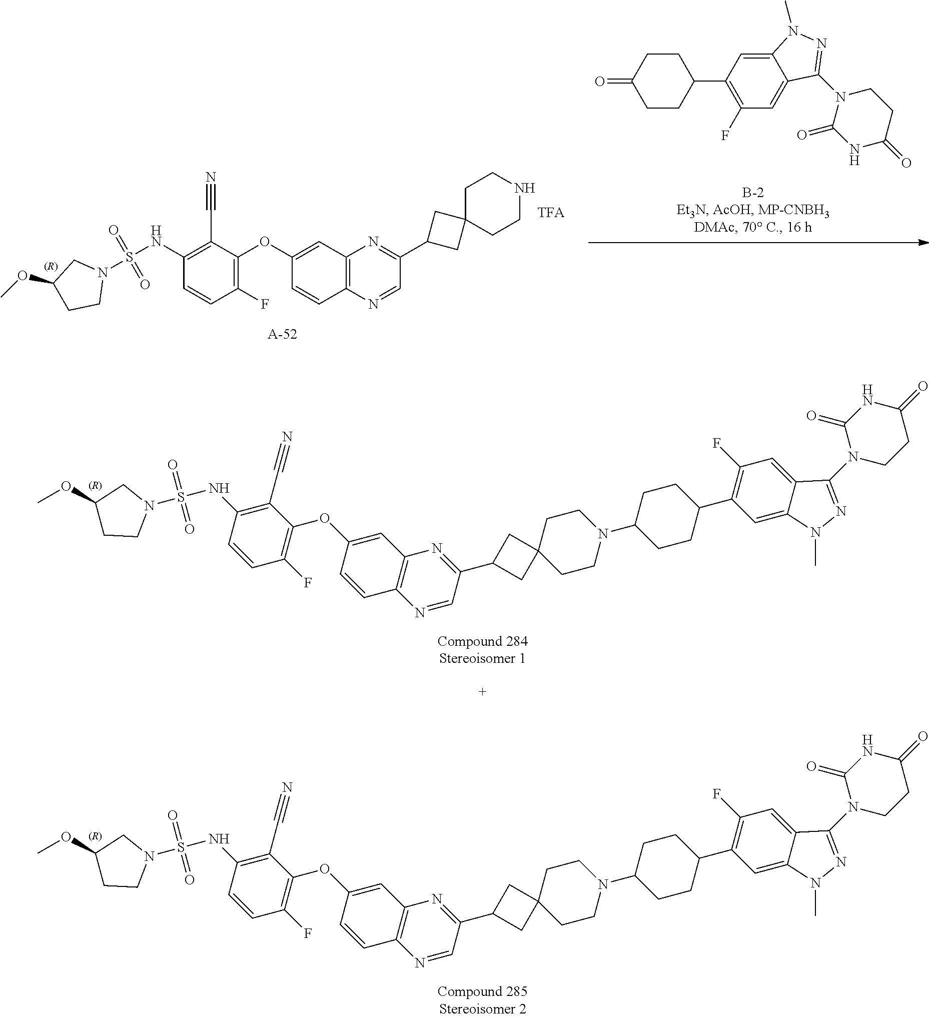

Into a 20 mL pressure vial containing a well-stirred solution of (3R)-N-[3-[3-(7-azaspiro[3.5]nonan-2-yl)quinoxalin-6-yl]oxy-2-cyano-4-fluoro-phenyl]-3-methoxy-pyrrolidine-1-sulfonamide (A-52) (260 mg, 381.98 mol, TFA salt) in anhydrous DMAc (6 mL) was added triethylamine (1.91 mmol, 266.20 μL) at room temperature and resulting mixture was stirred for min. To the above mixture, 1-[5-fluoro-1-methyl-6-(4-oxocyclohexyl)indazol-3-yl]hexahydropyrimidine-2,4-dione (B-2) (136.89 mg, 381.98 mol) and AcOH (1.15 mmol, 65.53 L) were added and stirred for 5 hours. Finally, MP-CNBH₃ (260 mg) was added and resulting reaction mixture was stirred at 70° C. for 16 hours. After completion of the reaction, the reaction mixture was filtered through sintered funnel, washed with dichloromethane and filtrate was concentrated under reduced pressure to get a crude residue, which was purified by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction (Peak-1) was collected and lyophilized to afford Compound 284 (assigned as stereoisomer 1).

Peak-1: (3R)-N-[2-cyano-3-[3-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-methoxy-pyrrolidine-1-sulfonamide (Compound 284, 16.07 mg, 17.54 mol, 5% yield) as an off-white solid. UPLC-MS (ES+): m/z 909.2 [M+H]⁺. ¹H NMR (400 MHz, DMSO-d₆): δ 10.56 (s, 1H), 8.77 (s, 1H), 8.12 (d, J=9.2 MHz, 1H), 7.68 (dd, J=2.8, 9.2 Hz, 1H), 7.54 (d, J=6.0 MHz, 1H), 7.47-7.31 (m, 3H), 7.14-7.04 (m, 1H), 3.99 (s, 3H), 3.96-3.88 (m, 4H), 3.31-3.27 (m, 2H), 3.19 (s, 3H), 3.17-2.84 (m, 7H), 2.75 (t, J=6.8 Hz, 2H), 2.32-2.10 (m, 7H), 2.09-1.92 (m, 5H), 1.82-1.53 (m, 7H).

Similarly, the second eluted fraction (Peak-2) was collected and lyophilized to afford Compound 285 (assigned as stereoisomer 2).

Peak-2: (3R)-N-[2-cyano-3-[3-[7-[4-[3-(2,4-dioxohexahydropyriimidin-1-yl)-5-fluoro-1-methyl-indazol-6-yl]cyclohexyl]-7-azaspiro[3.5]nonan-2-yl]quinoxalin-6-yl]oxy-4-fluoro-phenyl]-3-methoxy-pyrrolidine-1-sulfonamide (Compound 285, 29.06 mg, 31.82 mol, 8% yield) as an off-white solid. UPLC-MS (ES+): m/z 909.2 [M+H]⁺. ¹H NMR (400 MHz, DMSO-d₆): δ 10.56 (s, 1H), 8.77 (s, 1H), 8.52 (bs, 1H), 8.12 (d, J=9.2 MHz, 1H), 7.68 (d, J=6.8 Hz, 1H), 7.55-7.22 (m, 4H), 7.12-7.05 (m, 1H), 4.03 (s, 3H), 3.93-3.88 (m, 4H), 3.56-3.42 (m, 2H), 3.17 (s, 3H), 3.14-2.85 (m, 7H), 2.76 (t, J=6.8 Hz, 3H), 2.32-2.13 (m, 4H), 2.12-1.50 (m, 14H).

### Synthesis D177: Synthesis of (3S,5S)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 286 and Compound 287)

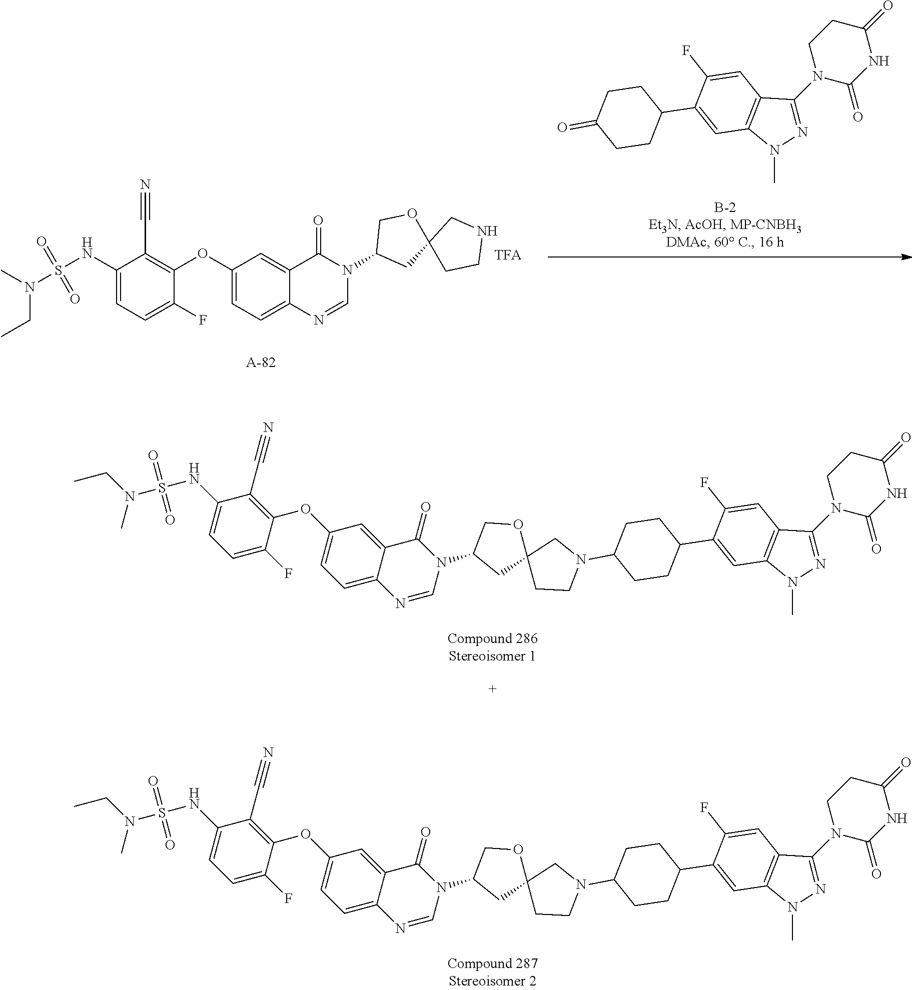

Into a 20 mL reaction vial containing a solution of (3S,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-82) (400.00 mg, 609.19 mol, TFA salt) in DMAc (5 mL) was added triethylamine (1.22 mmol, 169.82 μL) and stirred at room temperature for 10 min. Subsequently, AcOH (1.83 mmol, 104.52 μL) and 1-[5-fluoro-1-methyl-6-(4-oxocyclohexyl)indazol-3-yl]hexahydropyrimidine-2,4-dione (B-2) (261.98 mg, 731.03 mol) were added and stirred at room temperature for 6 hours. Finally, MP-CNBH₃ (300 mg, 609.19 mol) was added. The resultant reaction mixture was stirred at 60° C. for 16 hours. After completion, the reaction mixture was filtered through cotton pad and concentrated under reduced pressure to get crude residue which was purified by reverse phase column chromatography (Column: Redisep R_{f} Gold® reversed-phase C18-teledyne ISCO, 100 g, Mobile phase A: 10 mM NH₄HCO₃ in milli-Q water; Mobile phase B: acetonitrile). The first eluting fraction was collected and lyophilized to afford Peak-1 (Fast eluting isomer, 110 mg, assigned as stereoisomer 1) as an off-white solid (HPLC purity: 90%).

Similarly, the second eluting fraction was collected and lyophilized to afford Peak-2 (Late eluting isomer, 110 mg, assigned as stereoisomer 2) as an off-white solid (HPLC purity: 78%). The resultant Peak-1 (110 mg, HPLC purity: 90%) was further purified by preparative HPLC [Column: X BRIDGE C18 (150 mm*21 mm*5 μm), Mobile phase A: 10 mM ammonium bicarbonate (pH:10 adjust with ammonia in milli-Q water; Mobile phase B: Acetonitrile). The eluting fraction was collected and lyophilized to afford Compound 286 (assigned as stereoisomer 1).

Peak-1: (3S,5S)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 286, 50.4 mg, 56.33 mol, 9% yield) as an off-white solid. UPLC-MS (ES+): m/z 885.2 [M+H]⁺. ¹H NMR (400 MHz, DMSO-d₆): δ 10.56 (s, 1H), 9.98 (bs, 1H), 8.29 (s, 1H), 7.77 (d, J=8.80 Hz, 1H), 7.65 (dd, J=2.80, 8.80 Hz, 1H), 7.55 (d, J=6.00 Hz, 1H), 7.54-7.28 (m, 4H), 5.40-5.20 (m, 1H), 4.19-4.13 (m, 2H), 4.00 (s, 3H), 3.92-3.70 (m, 3H), 3.70-3.34 (m, 3H), 3.25-2.73 (m, 7H), 2.65-2.55 (m, 4H), 2.28-2.10 (m, 4H), 2.00-1.90 (m, 2H), 1.75-1.40 (m, 4H), 1.03 (t, J=7.20 Hz, 3H).

Similarly, the Peak-2 (110 mg, HPLC purity: 78%) was further purified by preparative HPLC [Column: X BRIDGE C18 (150 mm*21 mm*5 μm), Mobile phase A: 10 mM ammonium bicarbonate (pH:10 adjust with ammonia in milli-Q water; Mobile phase B: Acetonitrile). The eluting fraction was collected and lyophilized to afford Compound 287 (assigned as stereoisomer 2).

Peak-2: (3S,5S)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 287, 18.2 mg, 20.29 mol, 3% yield) as an off-white solid. UPLC-MS (ES+): m/z 885.2 [M+H]⁺. ¹H NMR (400 MHz, DMSO-d₆): δ 10.56 (s, 1H), 9.75 (bs, 1H), 8.30 (s, 1H), 7.79-7.66 (m, 3H), 7.50-7.35 (m, 4H), 5.40-5.20 (m, 1H), 4.30-3.80 (m, 8H), 3.80-3.35 (m, 3H), 3.20-2.75 (m, 8H), 2.65-2.55 (m, 3H), 2.20-1.50 (m, 10H), 1.04 (t, J=7.20 Hz, 3H).

### Synthesis D178: Synthesis of (3R,5R)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 288 and Compound 289)

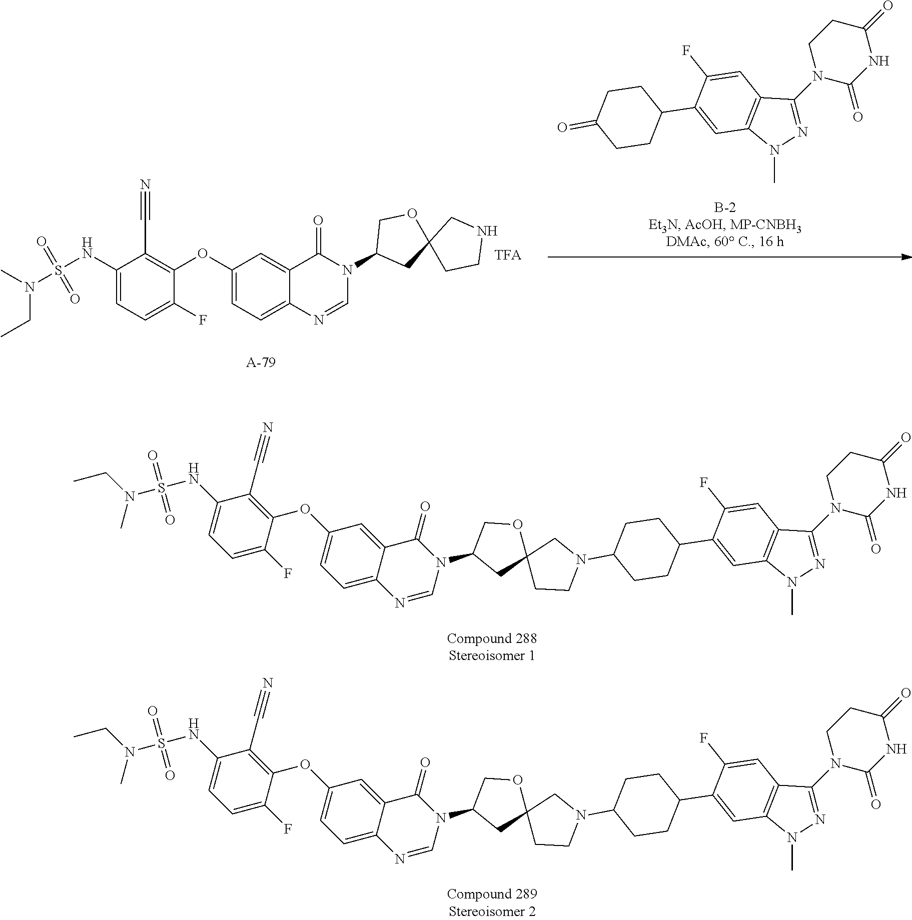

Into a 20 mL reaction vial containing a solution of (3R,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-79) (400 mg, 609.19 mol, TFA salt) in DMAc (5 mL) was added triethylamine (1.22 mmol, 169.82 μL) and stirred for 10 min at room temperature. Subsequently, AcOH (1.83 mmol, 104.52 μL) and 1-[5-fluoro-1-methyl-6-(4-oxocyclohexyl)indazol-3-yl]hexahydropyrimidine-2,4-dione (B-2) (261.98 mg, 731.03 mol) were added and stirred for 6 h at room temperature. Finally, MP-CNBH₃ (200 mg, 609.19 mol) was added and stirred at 60° C. for 16 hours. After completion, the reaction mixture was filtered through cotton pad and filtrate was concentrated to get a crude residue which was purified by reverse phase column chromatography (Column: Redisep R_{f} Gold® reversed-phase C18-teledyne ISCO, 100 g, Mobile phase A: 10 Mm NH₄HCO₃ in milli-Q water; Mobile phase B: acetonitrile). The eluted fractions were collected and lyophilized to afford Peak-1 (Fast eluting isomer, 100 mg, assigned as stereoisomer 1) as an off-white solid (HPLC purity: 73%) and Peak-2 (Late eluting isomer, 110 mg, assigned as stereoisomer 2) as an off-white solid (HPLC purity: 87%).

The resultant Peak-1 (100 mg, HPLC purity: 73%) was further purified by preparative HPLC [Column: X BRIDGE C18 (150 mm*21 mm*5 μm), Mobile phase A: 10 mM ABC (pH:10 adjust with ammonia in milli-Q water; Mobile phase B: Acetonitrile]. The eluting fraction was collected and lyophilized to afford Compound 288 (assigned as stereoisomer 1).

Peak-1: (3R,5R)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (38.82 mg, 43.49 mol, 7% yield) as an off-white solid. UPLC-MS (ES+): m/z 885.3 [M+H]⁺. ¹H NMR (400 MHz, DMSO-d₆): δ 10.56 (s, 1H), 10.03 (bs, 1H), 8.29 (s, 1H), 7.77 (d, J=8.80 Hz, 1H), 7.66 (dd, J=3.20, 9.00 Hz, 1H), 7.54 (d, J=5.60 Hz, 1H), 7.50-7.28 (m, 4H), 5.35-5.15 (m, 1H), 4.17-4.12 (m, 2H), 4.00 (s, 3H), 3.91 (t, J=6.80 Hz, 2H), 3.80-3.40 (m, 4H), 3.20-2.70 (m, 7H), 2.65-2.55 (m, 4H), 2.30-1.80 (m, 6H), 1.80-1.40 (m, 4H), 1.02 (t, J=7.20 Hz, 3H).

Similarly, the Peak-2 (110 mg, HPLC purity: 87%) was further purified by preparative HPLC [Column: X BRIDGE C18 (150 mm*21 mm*5 μm), Mobile phase A: 10 mM ABC (pH:10 adjust with ammonia in milli-Q water; Mobile phase B: Acetonitrile]. The eluting fraction was collected and lyophilized to afford Compound 289 (assigned as stereoisomer 2).

Peak-2: (3R,5R)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (30.66 mg, 33.82 mol, 5% yield) as an off-white solid. UPLC-MS (ES+): m/z 885.3 [M+H]⁺. ¹H NMR (400 MHz, DMSO-d₆): δ 10.56 (s, 1H), 9.76 (bs, 1H), 8.30 (s, 1H), 7.77 (d, J=8.80 Hz, 1H), 7.68-7.10 (m, 6H), 5.40-5.20 (m, 1H), 4.30-3.95 (m, 6H), 3.91 (t, J=6.80 Hz, 2H), 3.80-3.45 (m, 3H), 3.10-2.80 (m, 5H), 2.76 (t, J=6.80 Hz, 2H), 2.70-2.60 (m, 4H), 2.30-1.50 (m, 10H), 1.03 (t, J=7.20 Hz, 3H).

### Synthesis D179: Synthesis of (3S,5R)-3-[6-[2-Cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 290 and Compound 291)

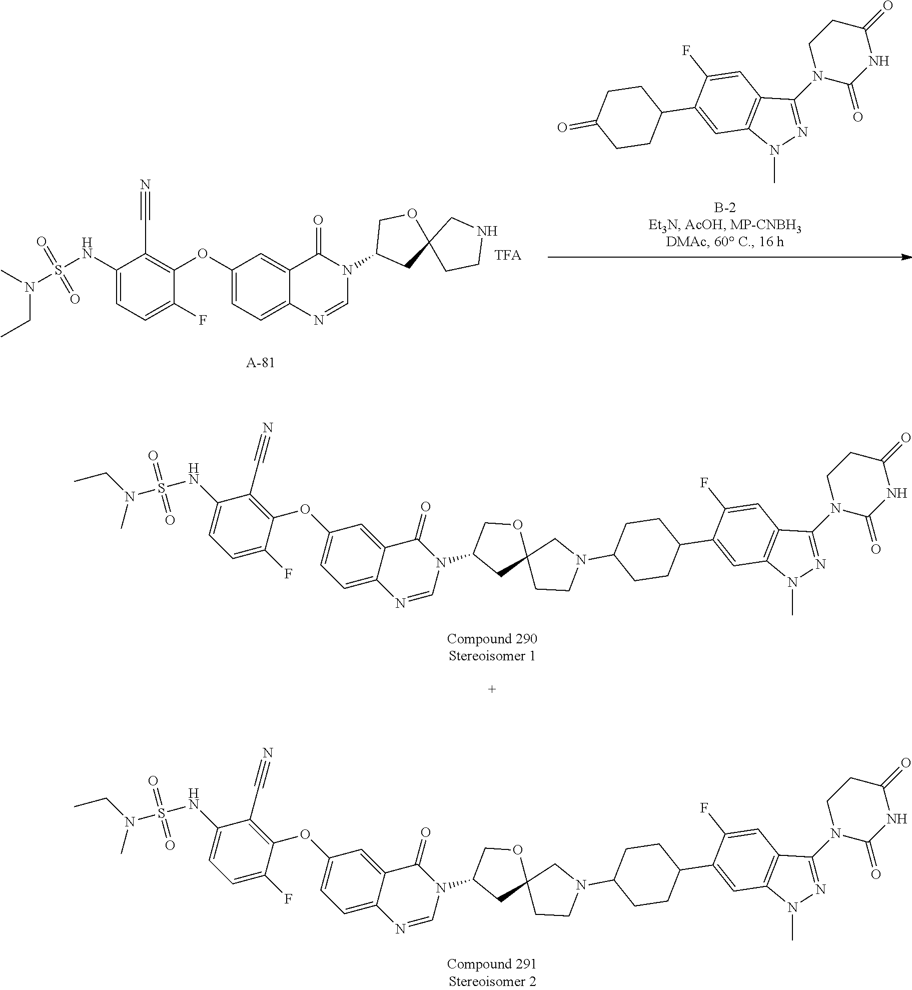

Into a 20 mL reaction vial containing a stirred solution of (3S,5R)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-81) (400.00 mg, 609.19 mol, TFA salt) in DMAc (5 mL) was added triethylamine (1.22 mmol, 169.82 μL) and stirred for 10 min at room temperature. Subsequently, AcOH (1.83 mmol, 104.52 μL) and 1-[5-fluoro-1-methyl-6-(4-oxocyclohexyl)indazol-3-yl]hexahydropyrimidine-2,4-dione (B-2) (261.98 mg, 731.03 mol) were added and stirred for 6 h at room temperature. Finally, MP-CNBH₃ (300 mg, 609.19 mol) was added and stirred at 60° C. for 16 hours. After completion, the reaction mixture was filtered and concentrated under reduced pressure to get the crude residue which was purified by reverse phase column chromatography (Column: Redisep R_{f} Gold® reversed-phase C18-teledyne ISCO, 60 g, Mobile phase A: 10 mM NH₄HCO₃ in milli-Q water; Mobile phase B: acetonitrile). The fractions were lyophilized to afford 500 mg of mixture of cis and trans isomers. Further cis and trans isomers were separated by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction (Peak-1) was collected and lyophilized to afford Compound 290 (assigned as stereoisomer 1).

Peak-1: (3S,5R)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 290, 65.71 mg, 73.49 mol, 12% yield) as an off-white solid. UPLC-MS (ES+): m/z 885.2 [M+H]⁺. ¹H NMR (400 MHz, DMSO-d₆): δ 10.56 (s, 1H), 10.02 (bs, 1H), 8.29 (s, 1H), 7.77 (d, J=8.80 Hz, 1H), 7.66 (dd, J=3.20, 9.00 Hz, 1H), 7.54 (d, J=6.00 Hz, 1H), 7.50-7.10 (m, 4H), 5.40-5.15 (m, 1H), 4.20-4.10 (m, 2H), 4.00 (s, 3H), 3.91 (t, J=6.40 Hz, 2H), 3.70-3.45 (m, 2H), 3.25-2.80 (m, 5H), 2.76 (t, J=6.80 Hz, 2H), 2.62-2.55 (m, 4H), 2.50-2.40 (m, 2H), 2.30-1.90 (m, 6H), 1.80-1.45 (m, 4H), 1.02 (t, J=7.20 Hz, 3H).

Similarly, the second eluted fraction (Peak-2) was collected and lyophilized to afford Compound 291 (assigned as stereoisomer 2).

Peak-2: (3S,5R)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 291, 51.63 mg, 58.07 mol, 10% yield) as an off-white solid. UPLC-MS (ES+): m/z 885.2 [M+H]⁺. ¹H NMR (400 MHz, DMSO-d₆): δ 10.56 (s, 1H), 10.18 (bs, 1H), 8.29 (s, 1H), 7.77 (d, J=8.80 Hz, 1H), 7.66 (dd, J=2.80, 8.80 Hz, 1H), 7.60-7.10 (m, 5H), 5.40-5.20 (m, 1H), 4.25-4.02 (m, 2H), 4.01 (s, 3H), 3.91 (t, J=6.40 Hz, 2H), 3.80-3.40 (m, 2H), 3.20-2.85 (m, 5H), 2.76 (t, J=6.80 Hz, 3H), 2.65-2.55 (m, 3H), 2.48-2.38 (m, 2H), 2.20-1.40 (m, 10H), 1.03 (t, J=7.20 Hz, 3H).

### Synthesis D180: Synthesis of (3R,5S)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 292 and Compound 293)

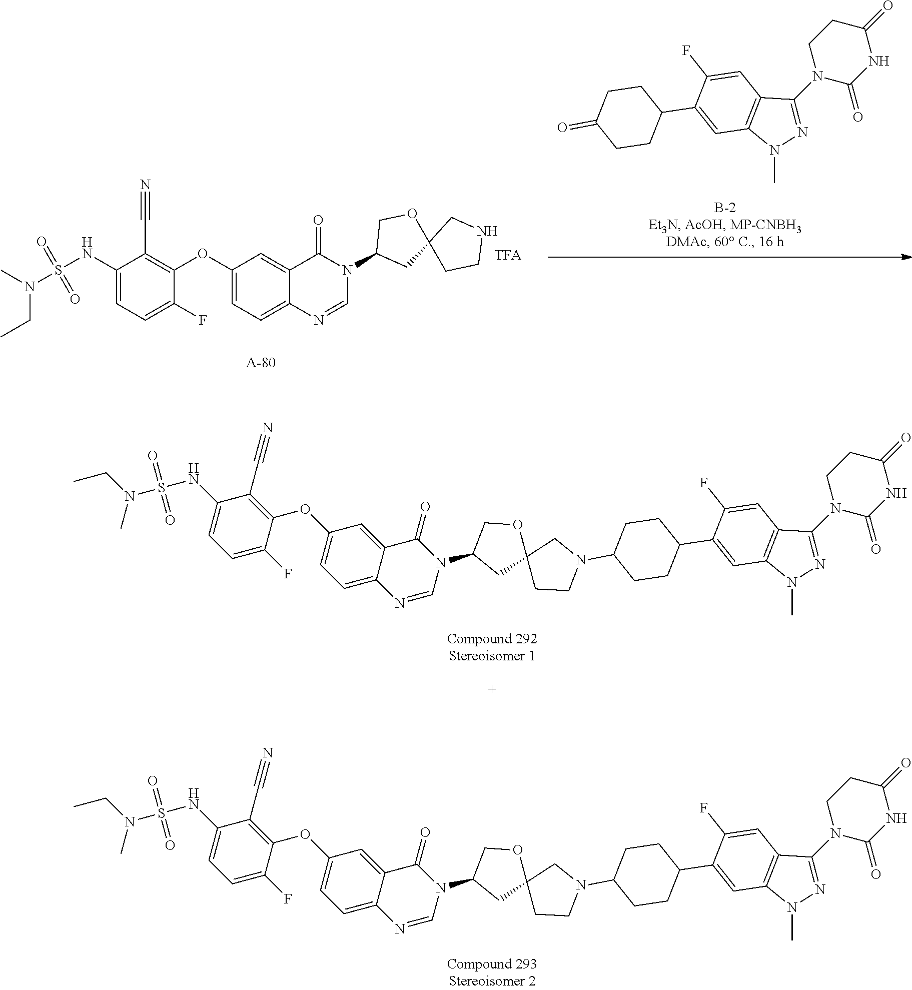

Into a 20 mL reaction vial containing a stirred solution of (3R,5S)-3-[6-[2-cyano-3-[[ethyl(methyl)sulfamoyl]amino]-6-fluoro-phenoxy]-4-oxo-quinazolin-3-yl]-1-oxa-7-azaspiro[4.4]nonane (A-80) (400 mg, 609.19 mol, TFA salt) in DMAc (5 mL) was added triethylamine (1.22 mmol, 169.82 μL) and stirred for 10 min at room temperature. Subsequently, AcOH (1.83 mmol, 104.52 μL) and 1-[5-fluoro-1-methyl-6-(4-oxocyclohexyl)indazol-3-yl]hexahydropyrimidine-2,4-dione (B-2) (261.98 mg, 731.03 mol) were added and stirred for 6 h at room temperature. Finally, MP-CNBH₃ (200 mg, 609.19 mol) was added and stirred at 60° C. for 16 hours. After completion, the reaction mixture was filtered through cotton pad and concentrated under reduced pressure to get the crude residue which was purified by reverse phase column chromatography (Column: Redisep R_{f} Gold® reversed-phase C18-teledyne ISCO, 60 g, Mobile phase A: 10 mM NH₄HCO₃ in milli-Q water; Mobile phase B: acetonitrile). The eluted fraction was collected and lyophilized to afford 300 mg of mixture of cis and trans isomers. Further cis and trans isomers were separated by preparative HPLC [Column: X-Bridge C18 (250 mm*19 mm*5 m), Mobile phase A: 10 mM NH₄HCO₃ in milli-Q water; Mobile phase B: acetonitrile]. The first eluting fraction (Peak-1) was collected and lyophilized to afford Compound 292 (assigned as stereoisomer 1).

Peak-1: (3R,5S)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 292, 89.55 mg, 100.39 mol, 16% yield) as an off-white solid. UPLC-MS (ES+): m/z 885.3 [M+H]⁺. ¹H NMR (400 MHz, DMSO-d₆): δ 10.56 (s, 1H), 9.98 (bs, 1H), 8.29 (s, 1H), 7.77 (d, J=8.80 Hz, 1H), 7.65 (dd, J=2.80, 8.80 Hz, 1H), 7.55 (d, J=6.00 Hz, 1H), 7.50-7.30 (m, 3H), 7.30-7.20 (m, 1H), 5.40-5.20 (m, 1H), 4.25-4.05 (m, 2H), 4.00 (s, 3H), 3.91 (t, J=6.80 Hz, 2H), 3.30-3.10 (m, 2H), 3.10-2.95 (m, 3H), 2.95-2.80 (m, 2H), 2.76 (t, J=6.80 Hz, 2H), 2.65-2.55 (m, 4H), 2.45-2.35 (m, 2H), 2.30-2.10 (m, 4H), 2.00-1.90 (m, 2H), 1.75-1.35 (m, 4H), 1.03 (t, J=7.20 Hz, 3H).

Similarly, the second eluted fraction (Peak-2) was collected and lyophilized to afford Compound 293 (assigned as stereoisomer 2).

Peak-2: (3R,5S)-3-[6-[2-cyano-3-[[ethyl(methyl)sulfamoyl]amino]-6-fluoro-phenoxy]-4-oxo-quinazolin-3-yl]-7-[4-[3-(2,4-dioxohexahydropyrimidin-1-yl)-5-fluoro-1-methyl-indazol-6-yl]cyclohexyl]-1-oxa-7-azaspiro[4.4]nonane (Compound 293, 64.57 mg, 72.26 mol, 12% yield) as an off-white solid. UPLC-MS (ES+): m/z 885.3 [M+H]⁺. ¹H NMR (400 MHz, DMSO-d₆): δ 10.56 (s, 1H), 9.90 (bs, 1H), 8.30 (s, 1H), 7.78 (d, J=8.80 Hz, 1H), 7.67 (dd, J=2.80, 8.80 Hz, 1H), 7.60-7.50 (m, 1H), 7.45 (d, J=6.00 Hz, 1H), 7.40-7.30 (m, 3H), 5.40-5.20 (m, 1H), 4.20-4.07 (m, 2H), 4.01 (s, 3H), 3.91 (t, J=6.40 Hz, 2H), 3.70-3.40 (m, 2H), 3.20-2.90 (m, 4H), 2.76 (t, J=6.40 Hz, 3H), 2.70-2.60 (m, 5H), 2.25-1.45 (m, 11H), 1.04 (t, J=7.20 Hz, 3H).

### Synthesis D181: Synthesis of 7-[2-cyano-6-fluoro-3-[[2-hydroxyethyl(methyl)sulfamoyl]amino]phenoxy]-2-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxaline (Compound 294 and Compound 295)

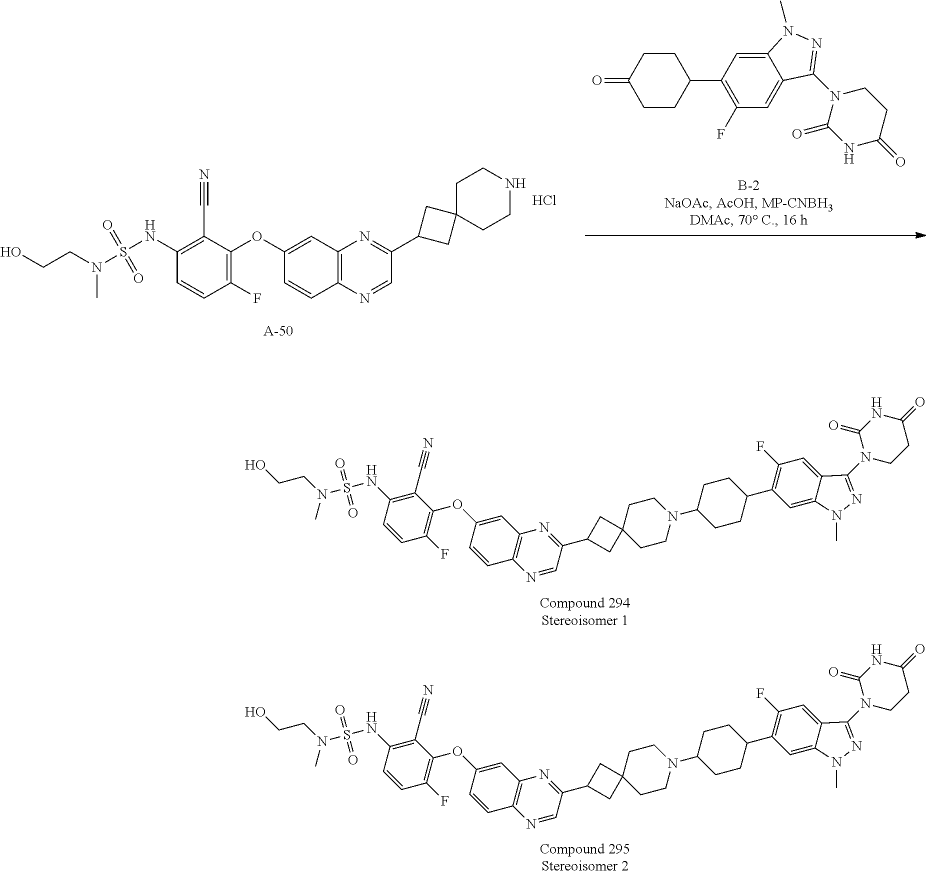

Into a 20 mL pressure vial containing a well-stirred solution of 2-(7-azaspiro[3.5]nonan-2-yl)-7-[2-cyano-6-fluoro-3-[[2-hydroxyethyl(methyl)sulfamoyl]amino]phenoxy]quinoxaline (A-50) (200 mg, 235.25 mol, HCl salt) in anhydrous DMAc (3 mL) was added NaOAc (96.49 mg, 1.18 mmol) at room temperature and stirred for 30 min. To the above mixture, 1-[5-fluoro-1-methyl-6-(4-oxocyclohexyl)indazol-3-yl]hexahydropyrimidine-2,4-dione (B-2) (110.70 mg, 305.82 mol) and AcOH (705.74 mol, 40.36 μL) were added at room temperature and stirred for 1 hour. Finally, MP-CNBH₃ (200 mg) was added and resulting reaction mixture was stirred at 70° C. for 16 hours. After completion of the reaction, the reaction mixture was filtered through sintered funnel, washed with dichloromethane and filtrate was concentrated under reduced pressure to get a crude residue which was purified by the preparative HPLC [Column: X-Bridge C18 (150 mm*19 mm*5 μm), Mobile phase A: 10 mM NH₄HCO₃ in milli-Q water; Mobile phase B: acetonitrile]. The first eluting fraction (Peak-1) was lyophilized to afford Compound 294 (assigned as stereoisomer 1).

Peak-1: 7-[2-cyano-6-fluoro-3-[[2-hydroxyethyl(methyl)sulfamoyl]amino]phenoxy]-2-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxaline (Compound 294, 11.42 mg, 12.47 mol, 5% yield) as an off-white solid. UPLC-MS (ES+): m/z 883.2 [M+H]⁺. ¹H NMR (400 MHz, DMSO-d₆): δ 10.56 (s, 1H), 8.94 (bs, 1H), 8.78 (s, 1H), 8.14 (d, J=3.20 Hz, 1H), 7.68 (dd, J=2.80, 9.20 Hz, 1H), 7.54 (d, J=6.00 Hz, 1H), 7.40-7.25 (m, 3H), 7.09-7.08 (m, 1H), 4.60-4.50 (m, 1H), 3.99 (s, 3H), 3.96-3.89 (m, 3H), 3.60-3.48 (m, 3H), 3.25-2.88 (m, 7H), 2.76 (t, J=6.80 Hz, 2H), 2.65 (s, 3H), 2.29-2.23 (m, 3H), 2.20-2.12 (m, 3H), 2.08-1.60 (m, 10H).

Similarly, the second eluted fraction (Peak-2) was lyophilized to afford Compound 295 (assigned as stereoisomer 2).

Peak-2: 7-[2-cyano-6-fluoro-3-[[2-hydroxyethyl(methyl)sulfamoyl]amino]phenoxy]-2-[7-[4-[3-(2,4-dioxohexahydropyrimidin-1-yl)-5-fluoro-1-methyl-indazol-6-yl]cyclohexyl]-7-azaspiro[3.5]nonan-2-yl]quinoxaline (Compound 295, 10.39 mg, 11.58 mol, 5% yield) as an off-white solid. UPLC-MS (ES+): m/z 883.3 [M+H]⁺. ¹H NMR (400 MHz, DMSO-d₆): δ 10.56 (s, 1H), 8.77 (s, 1H), 8.54 (bs, 1H), 8.12 (d, J=9.20 Hz, 1H), 7.68 (dd, J=2.40, 9.20 Hz, 1H), 7.50-7.25 (m, 4H), 7.10 (s, 1H), 4.62-4.46 (m, 1H), 4.03 (s, 3H), 3.92 (t, J=6.80 Hz, 3H), 3.55-3.38 (m, 4H), 3.28-2.85 (m, 5H), 2.76 (t, J=6.80 Hz, 3H), 2.68 (s, 3H), 2.30-2.18 (m, 4H), 2.10-1.70 (m, 9H).

### Synthesis D182: Synthesis of 6-[2-cyano-3-[[ethyl(methyl)sulfamoyl]amino]-6-fluoro-phenoxy]-3-[2-[[1-[3-(2,4-dioxohexahydropyrimidin-1-yl)-5-fluoro-1-methyl-indazol-6-yl]-4-piperidyl]methyl]-2-azaspiro[3.3]heptan-6-yl]-4-oxo-quinazoline (Compound 296)

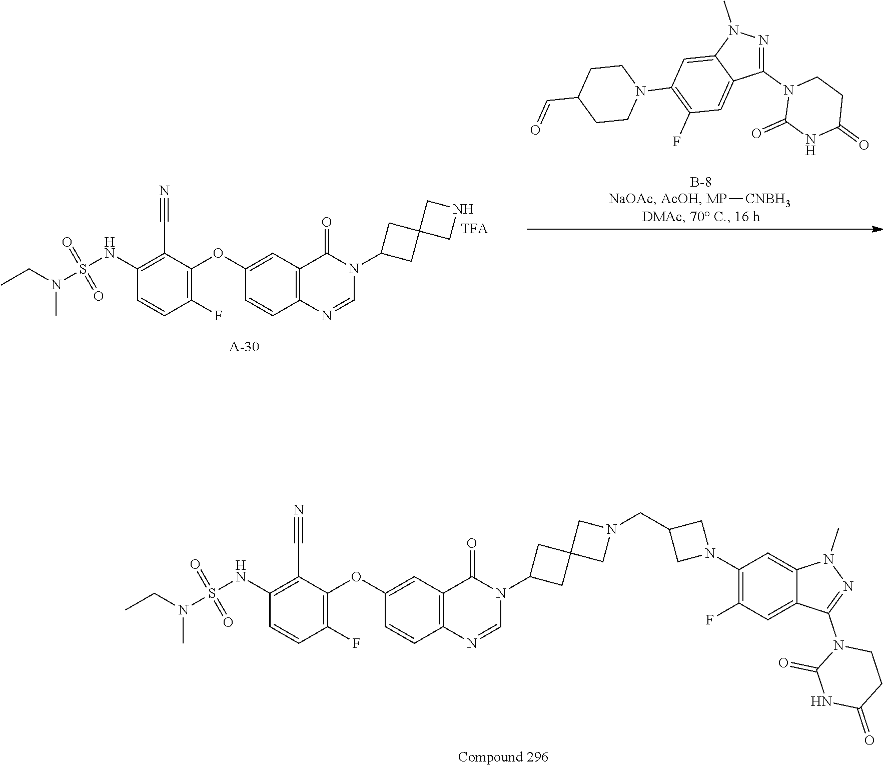

Into a 20 mL pressure vial containing a well-stirred solution of 3-(2-azaspiro[3.3]heptan-6-yl)-6-[2-cyano-3-[[ethyl(methyl)sulfamoyl]amino]-6-fluoro-phenoxy]-4-oxo-quinazoline (A-30) (135.80 mg, 214.61 mol, TFA salt) in anhydrous DMAc (2 mL) was added NaOAc (88.02 mg, 1.07 mmol) at room temperature and resulting mixture was stirred for 30 min. To the above mixture, 1-[3-(2,4-dioxohexahydropyrimidin-1-yl)-5-fluoro-1-methyl-indazol-6-yl]piperidine-4-carbaldehyde (B-8) (84.35 mg, 214.61 mol) and AcOH (643.83 mol, 36.81 μL) were added at room temperature and mixture was stirred for 5 hours. Finally, MP-CNBH₃ (110 mg) was added and stirred at 70° C. for 16 hours. After completion of the reaction, the reaction mixture was filtered through sintered funnel, washed with dichloromethane and filtrate was concentrated under reduced pressure to get a crude residue, which was purified by reverse phase column chromatography (Column: Redisep R_{f} Gold®reversed-phase C18-teledyne ISCO, 120 g), [Mobile phase A: 10 mM NH₄HCO₃ in milli-Q water; Mobile phase B: acetonitrile]. Fractions were lyophilized to afford 6-[2-cyano-3-[[ethyl(methyl)sulfamoyl]amino]-6-fluoro-phenoxy]-3-[2-[[1-[3-(2,4-dioxohexahydropyrimidin-1-yl)-5-fluoro-1-methyl-indazol-6-yl]-4-piperidyl]methyl]-2-azaspiro[3.3]heptan-6-yl]-4-oxo-quinazoline, which is not pure. Further purified by the preparative HPLC [Column: X-Bridge C8 (250 mm*19.5 mm*5 m), Mobile Phase A: 10 mM NH₄HCO₃ in milli-Q water; Mobile phase B: acetonitrile] to afford 6-[2-cyano-3-[[ethyl(methyl)sulfamoyl]amino]-6-fluoro-phenoxy]-3-[2-[[1-[3-(2,4-dioxohexahydropyrimidin-1-yl)-5-fluoro-1-methyl-indazol-6-yl]-4-piperidyl]methyl]-2-azaspiro[3.3]heptan-6-yl]-4-oxo-quinazoline (Compound 296, 37.31 mg, 42.58 mol, 20% yield) as an off-white solid. UPLC-MS (ES+): m/z 870.2 [M+H]⁺. ¹H NMR (400 MHz, DMSO-d₆): δ 10.53 (s, 1H), 9.63 (bs, 1H), 8.34 (s, 1H), 7.76 (d, J=8.80 Hz, 1H), 7.64 (dd, J=3.20, 9.20 Hz, 1H), 7.36-7.22 (m, 4H), 7.11 (d, J=6.80 Hz, 1H), 4.82-4.72 (m, 1H), 4.45-4.00 (m, 3H), 3.95 (s, 3H), 3.89 (t, J=6.8 Hz, 2H), 3.48-3.41 (m, 2H), 3.14-2.95 (m, 4H), 2.89-2.63 (m, 9H), 2.57 (s, 3H), 1.91-1.60 (m, 3H), 1.45-1.35 (m, 2H), 1.03 (t, J=7.20 Hz, 3H).

### Synthesis D183: Synthesis of 7-[2-cyano-3-[[ethyl(methyl)sulfamoyl]amino]-6-fluoro-phenoxy]-2-[2-[4-[3-(2,4-dioxohexahydropyrimidin-1-yl)-5-fluoro-1-methyl-indazol-6-yl]cyclohexyl]-2-azaspiro[3.3]heptan-6-yl]quinoxaline (Compound 297 and Compound 298)

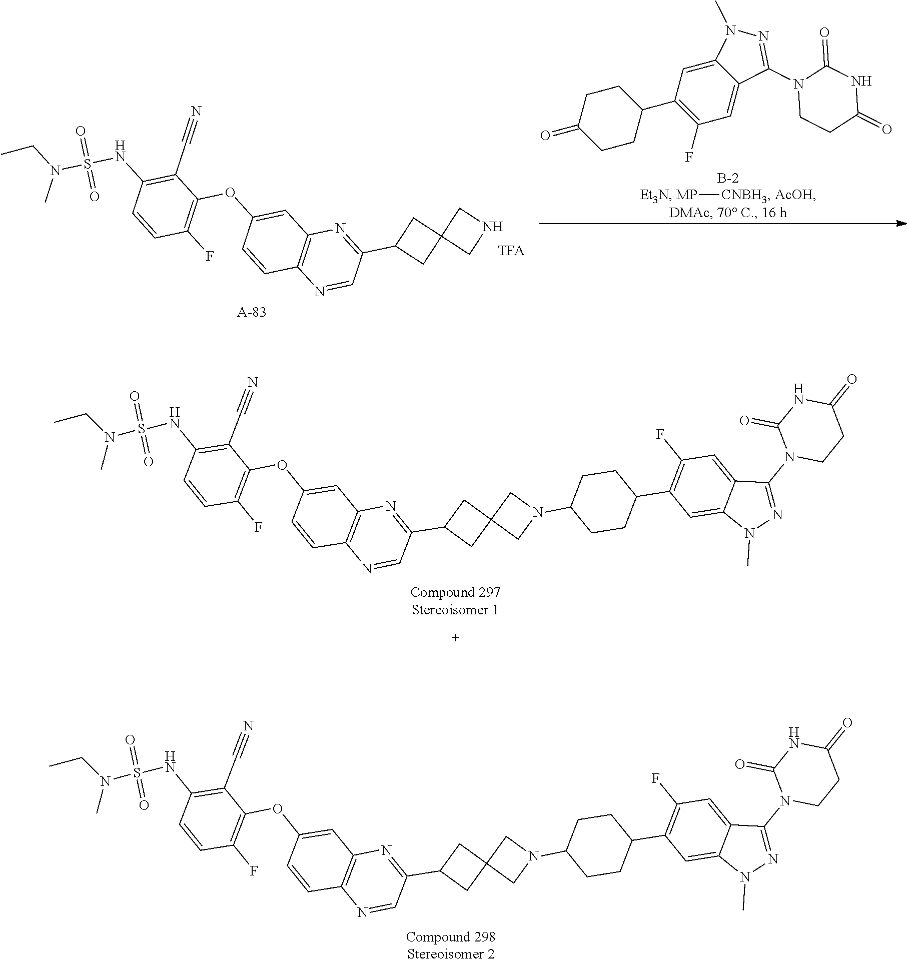

Into a 40 mL glass vial with pressure released cap containing a well-stirred solution of 2-(2-azaspiro[3.3]heptan-6-yl)-7-[2-cyano-3-[[ethyl(methyl)sulfamoyl]amino]-6-fluoro-phenoxy]quinoxaline (A-83) (300 mg, 375.68 mol, TFA salt) in DMAc (5 mL) was added triethylamine (1.13 mmol, 157.08 μL) at room temperature and stirred for 30 min. Subsequently, 1-[5-fluoro-1-methyl-6-(4-oxocyclohexyl)indazol-3-yl]hexahydropyrimidine-2,4-dione (B-2) (139.61 mg, 375.68 mol) and AcOH (751.35 mol, 42.97 μL) were added to the reaction mixture and stirred at room temperature for 2 hours. Finally, MP-CNBH₃ (300 mg, 375.68 mol) was added, and stirred at 70° C. for 16 hours. After completion of the reaction, the reaction mixture was concentrated under reduced pressure to get a crude residue (Mixture of cis and trans isomers), which further purified by Perp HPLC [Column: X-Bridge C8 (250 mm*19 mm*5 m), Mobile phase A: 10 mM NH₄HCO₃ in milli-Q water; Mobile phase B: acetonitrile]. The first eluting fraction at t_{R}=5.12 min by HPLC was concentrated under reduced pressure at 40° C. and lyophilized to afford Compound 297 (assigned as stereoisomer 1).

Peak-1: 7-[2-cyano-3-[[ethyl(methyl)sulfamoyl]amino]-6-fluoro-phenoxy]-2-[2-[4-[3-(2,4-dioxohexahydropyrimidin-1-yl)-5-fluoro-1-methyl-indazol-6-yl]cyclohexyl]-2-azaspiro[3.3]heptan-6-yl]quinoxaline (Compound 297, 30.3 mg, 34.37 mol, 9% yield) as an off-white solid. UPLC-MS (ES+): m/z 839.2 [M+H]⁺. ¹H NMR (400 MHz, DMSO-d₆): δ 10.56 (s, 1H), 9.80 (bs, 1H), 8.76 (s, 1H), 8.13 (d, J=9.20 Hz, 1H), 7.69 (dd, J=2.80, 9.20 Hz, 1H), 7.53 (d, J=6.00 Hz, 1H), 7.40-7.37 (m, 2H), 7.27 (dd, J=4.00, 9.20 Hz, 1H), 7.12 (d, J=2.80 Hz, 1H), 4.44-4.05 (m, 2H), 4.00 (s, 3H), 3.92-3.81 (m, 3H), 3.20-3.06 (m, 1H), 2.99 (q, J=7.20 Hz, 2H), 2.90-2.80 (m, 1H), 2.76 (t, J=6.80 Hz, 2H), 2.70-2.61 (m, 2H), 2.57 (s, 3H), 2.10-1.85 (m, 5H), 1.68-1.54 (m, 2H), 1.38-1.20 (m, 2H), 1.03 (t, J=7.20 Hz, 3H).

Similarly, the second eluted fraction at t_{R}=5.37 min by HPLC was concentrated under reduced pressure at 40° C. and lyophilized to afford Compound 298 (assigned as stereoisomer 2).

Peak-2: 7-[2-cyano-3-[[ethyl(methyl)sulfamoyl]amino]-6-fluoro-phenoxy]-2-[2-[4-[3-(2,4-dioxohexahydropyrimidin-1-yl)-5-fluoro-1-methyl-indazol-6-yl]cyclohexyl]-2-azaspiro[3.3]heptan-6-yl]quinoxaline (Compound 298, 17.2 mg, 20.36 mol, 5% yield) as an off-white solid. UPLC-MS (ES+): m/z 839.2 [M+H]⁺. ¹H NMR (400 MHz, DMSO-d₆): δ 10.56 (s, 1H), 9.49 (bs, 1H), 8.78 (s, 1H), 8.13 (d, J=9.20 Hz, 1H), 7.68 (dd, J=2.80, 8.20 Hz, 1H), 7.44-7.30 (m, 4H), 7.15 (d, J=2.00 Hz, 1H), 4.43-4.12 (m, 3H), 4.01 (s, 3H), 3.91 (t, J=6.80 Hz, 2H), 3.83 (t, J=8.40 Hz, 1H), 3.61-3.49 (m, 1H), 3.06-2.91 (m, 3H), 2.86-2.71 (m, 4H), 2.62-2.54 (m, 5H), 2.00-1.52 (m, 9H), 1.03 (t, J=7.20 Hz, 3H).

### Synthesis D184: Synthesis of 6-[2-cyano-3-[[ethyl(methyl)sulfamoyl]amino]-6-fluoro-phenoxy]-3-[2-[4-[3-(2,4-dioxohexahydropyrimidin-1-yl)-5-fluoro-1-methyl-indazol-6-yl]-2-fluoro-cyclohexyl]-2-azaspiro[3.3]heptan-6-yl]-4-oxo-quinazoline (Compound 299) and 6-[2-cyano-3-[[ethyl(methyl)sulfamoyl]amino]-6-fluoro-phenoxy]-3-[2-[(4-[3-(2,4-dioxohexahydropyrimidin-1-yl)-5-fluoro-1-methyl-indazol-6-yl]-2-fluoro-cyclohexyl]-2-azaspiro[3.3]heptan-6-yl]-4-oxo-quinazoline (Compound 300)

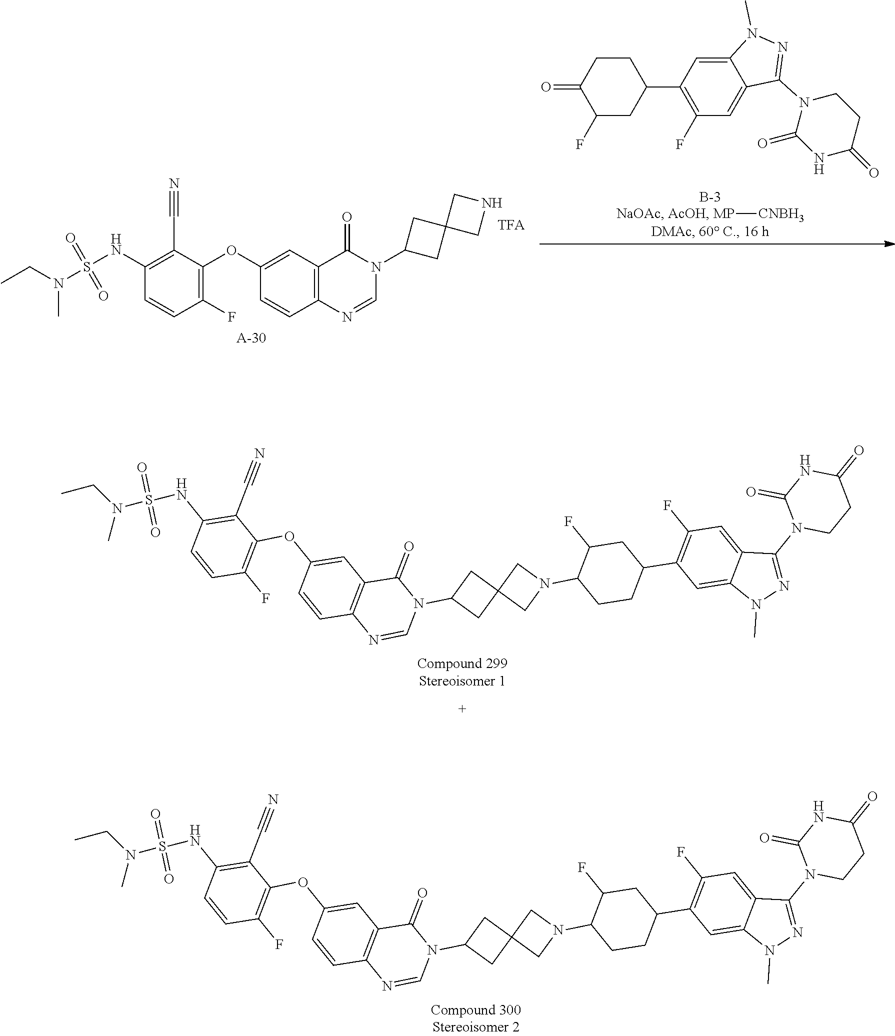

Into a 20 mL pressure vial containing a well-stirred solution of 6-(2-azaspiro[3.3]heptan-6-yl)-6-[2-cyano-3-[[ethyl(methyl)sulfamoyl]amino]-6-fluoro-phenoxy]-4-oxo-quinazoline (A-30) (350 mg, 558.59 mol, TFA salt) in anhydrous DMAc (5 mL) was added NaOAc (229.12 mg, 2.79 mmol) at room temperature and resulting mixture was stirred for 30 min. To the above mixture, 1-[5-fluoro-6-(3-fluoro-4-oxo-cyclohexyl)-1-methyl-indazol-3-yl]hexahydropyrimidine-2,4-dione (B-3) (252.27 mg, 670.31 mol) and AcOH (1.68 mmol, 95.84 μL) were stirred for 5 hours. Subsequently, MP-CNBH₃ (525 mg) was added and resulting reaction mixture was stirred at 60° C. for 16 hours. After completion of the reaction, the reaction mixture was filtered through sintered funnel, washed with dichloromethane and filtrate was concentrated under reduced pressure to get a crude residue which was purified by reverse phase column chromatography (Column: Redisep R_{f} Gold®reversed-phase C18-teledyne ISCO, 60 g), [Mobile phase A:10 mM NH₄HCO₃ in milli-Q water; Mobile phase B: acetonitrile]. The first eluting fraction (Peak-1) was lyophilized to afford Compound 299 (assigned as stereoisomer 1).

Peak-1: 6-[2-cyano-3-[[ethyl(methyl)sulfamoyl]amino]-6-fluoro-phenoxy]-3-[2-[(1R,4R)-4-[3-(2,4-dioxohexahydropyrimidin-1-yl)-5-fluoro-1-methyl-indazol-6-yl]-2-fluoro-cyclohexyl]-2-azaspiro[3.3]heptan-6-yl]-4-oxo-quinazoline (Compound 299, 17 mg, 18.76 mol, 3% yield) as an off-white solid. UPLC-MS (ES+): m/z 873.3 [M+H]⁺. ¹H NMR (400 MHz, DMSO-d₆): δ 10.56 (s, 1H), 10.24 (bs, 1H), 8.36 (s, 1H), 7.77 (d, J=8.80 Hz, 1H), 7.66-7.50 (m, 3H), 7.41-7.30 (m, 3H), 4.85-4.53 (m, 2H), 4.01 (s, 3H), 3.91 (t, J=6.80 Hz, 2H), 3.80-3.52 (m, 2H), 3.25-3.20 (m, 2H), 3.13-3.05 (m, 3H), 2.77-2.65 (m, 8H), 2.45-2.35 (m, 2H), 2.25-2.15 (m, 1H), 2.02-1.82 (m, 3H), 1.56-1.47 (m, 1H), 1.30-1.18 (m, 1H), 1.04 (t, J=14.40 Hz, 3H).

Similarly, the second eluting fraction (Peak-2) was lyophilized to afford Compound 300 (assigned as stereoisomer 2).

Peak-2: 6-[2-cyano-3-[[ethyl(methyl)sulfamoyl]amino]-6-fluoro-phenoxy]-3-[2-[(1R,4S)-4-[3-(2,4-dioxohexahydropyrimidin-1-yl)-5-fluoro-1-methyl-indazol-6-yl]-2-fluoro-cyclohexyl]-2-azaspiro[3.3]heptan-6-yl]-4-oxo-quinazoline (Compound 300.49 mg, 54.18 mol, 10% yield) as an off-white solid. UPLC-MS (ES+): m/z 873.4 [M+H]⁺. ¹H NMR (400 MHz, DMSO-d₆): δ 10.56 (s, 1H), 10.24 (bs, 1H), 8.38 (s, 1H), 7.78 (d, J=8.80 Hz, 1H), 7.68-7.50 (m, 3H), 7.41-7.30 (m, 3H), 4.85-4.54 (m, 2H), 4.03 (s, 3H), 3.91 (t, J=6.80 Hz, 2H), 3.70-3.52 (m, 2H), 3.25-3.20 (m, 2H), 3.10-3.01 (m, 3H), 2.77-2.51 (m, 8H), 2.45-2.38 (m, 2H), 2.25-2.15 (m, 1H), 2.00-1.80 (m, 3H), 1.55-1.46 (m, 1H), 1.31-1.15 (m, 1H), 1.05 (t, J=7.20 Hz, 3H).

### Synthesis D185: Synthesis of 6-[2-cyano-3-[[ethyl(methyl)sulfamoyl]amino]-6-fluoro-phenoxy]-3-[6-[2-[3-(2,4-dioxohexahydropyrimidin-1-yl)-5-fluoro-1-methyl-indazol-6-yl]-2-azaspiro[3.3]heptan-6-yl]-6-azaspiro[3.4]octan-2-yl]-4-oxo-quinazoline (Compound 301 and Compound 302)

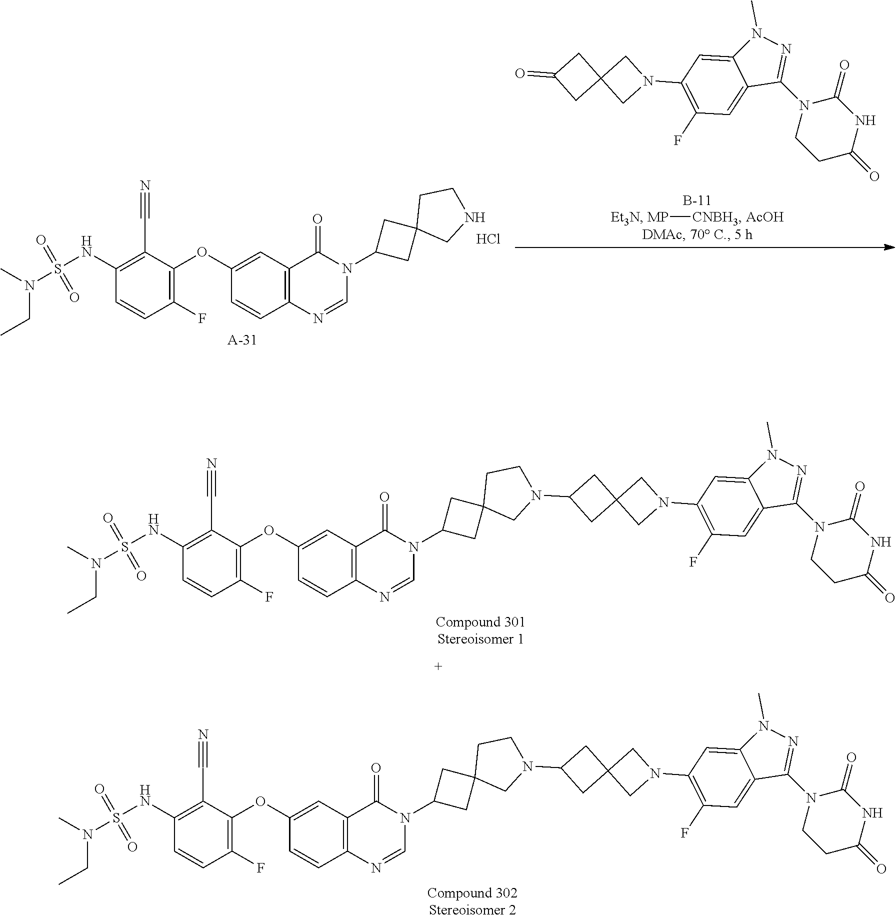

Into a 20 mL glass vial with pressure released cap containing a well-stirred solution of 3-(6-azaspiro[3.4]octan-2-yl)-6-[2-cyano-3-[[ethyl(methyl)sulfamoyl]amino]-6-fluoro-phenoxy]-4-oxo-quinazoline (A-31) (100 mg, 177.61 mol, HCl salt) in anhydrous DMAc (3 mL) was added 1-[5-fluoro-1-methyl-6-(6-oxo-2-azaspiro[3.3]heptan-2-yl)indazol-3-yl]hexahydropyrimidine-2,4-dione (B-11) (65.96 mg, 177.61 mol) at room temperature and mixture was stirred for 10 min. To the above mixture, triethylamine (888.03 mol, 123.77 μL) and AcOH (355.21 mol, 20.31 μL) was added and stirred at room temperature for 2 hours. Finally, MP-CNBH₃ (350 mg, 530.61 mol) was added at the same temperature and resulting reaction mixture was stirred at 70° C. for 5 hours. After completion of the reaction, the reaction mixture was filtered through cotton pad and filtrate was concentrated under reduced pressure to get a crude residue which was purified by preparative HPLC [Column: X-BRIDGE C18 (150 mm*19 mm*5 μm), Mobile Phase A: 10 mM NH₄HCO₃ in milli-Q water and NH₄OH (pH 10); Mobile phase B: acetonitrile]. The first eluting fraction (Peak-1 by Prep HPLC) was lyophilized to afford:

Peak-1: 6-[2-cyano-3-[[ethyl(methyl)sulfamoyl]amino]-6-fluoro-phenoxy]-3-[6-[2-[3-(2,4-dioxohexahydropyrimidin-1-yl)-5-fluoro-1-methyl-indazol-6-yl]-2-azaspiro[3.3]heptan-6-yl]-6-azaspiro[3.4]octan-2-yl]-4-oxo-quinazoline (Compound 301, 9 mg, 10.18 mol, 6% yield) as an off-white solid. Assigned as stereoisomer 1. UPLC-MS (ES+): m/z 882.2 [M+H]⁺. ¹H NMR (400 MHz, DMSO-d₆): δ 10.50 (s, 1H), 9.99 (bs, 1H), 8.37 (s, 1H), 7.77 (d, J=9.20 Hz, 1H), 7.66-7.47 (m, 2H), 7.33-0.23 (m, 3H), 6.51 (d, J=7.60 Hz, 1H), 4.99-4.90 (m, 1H), 4.01-3.74 (m, 10H), 3.14-3.00 (m, 4H), 2.73-2.61 (m, 9H), 2.39-2.32 (m, 2H), 2.20-2.15 (m, 2H), 1.03 (t, J=6.80 Hz, 3H).

Similarly, the second eluting fraction (Peak-2 by Prep HPLC) was lyophilized to afford Compound 302.

Peak-2: 6-[2-cyano-3-[[ethyl(methyl)sulfamoyl]amino]-6-fluoro-phenoxy]-3-[6-[2-[3-(2,4-dioxohexahydropyrimidin-1-yl)-5-fluoro-1-methyl-indazol-6-yl]-2-azaspiro[3.3]heptan-6-yl]-6-azaspiro[3.4]octan-2-yl]-4-oxo-quinazoline (Compound 302, 6 mg, 6.64 mol, 4% yield) as a pale yellow solid. Assigned as stereoisomer 2. UPLC-MS (ES+): m/z 882.2 [M+H]⁺. ¹H NMR (400 MHz, DMSO-d₆): δ 10.51 (s, 1H), 9.91 (bs, 1H), 8.37 (s, 1H), 7.77 (d, J=8.80 Hz, 1H), 7.64 (dd, J=3.20, 9.00 Hz, 1H), 7.45-7.25 (m, 4H), 6.52 (d, J=7.60 Hz, 1H), 4.90-4.86 (m, 1H), 4.02 (s, 2H), 3.89 (s, 2H), 3.89-3.86 (m, 5H), 3.18-2.99 (m, 2H), 2.73 (t, J=6.80 Hz, 2H), 2.68-2.56 (m, 6H), 2.38-2.31 (m, 2H), 2.18-2.06 (m, 2H), 1.03 (t, J=7.20 Hz, 3H).

### Synthesis D186: Synthesis of 7-[2-cyano-3-[[ethyl(methyl)sulfamoyl]amino]-6-fluoro-phenoxy]-2-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]quinoxaline (Compound 303)

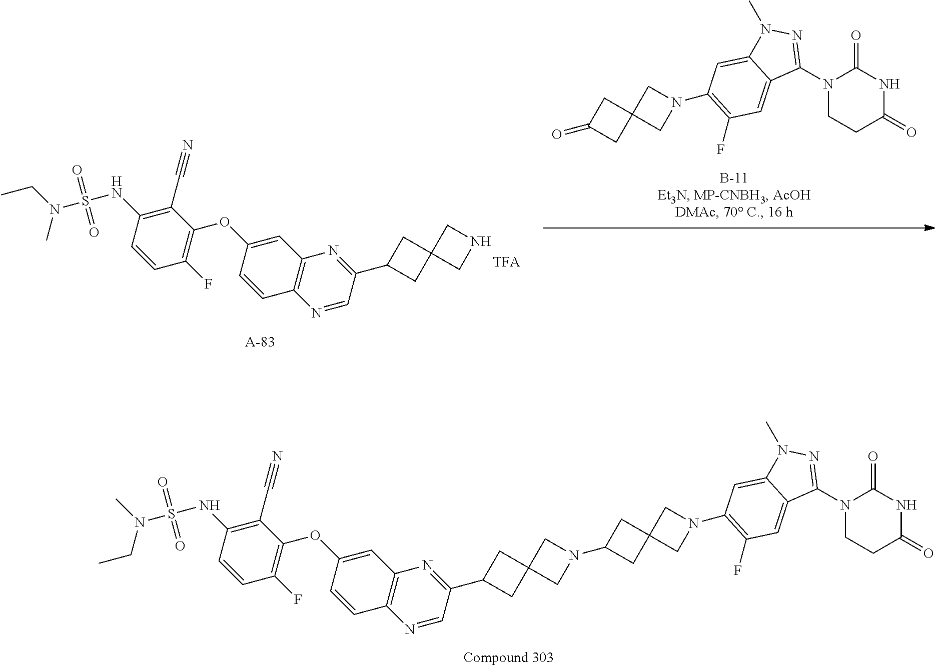

Into a 30 mL glass vial containing a well stirred solution of 2-(2-azaspiro[3.3]heptan-6-yl)-7-[2-cyano-3-[[ethyl(methyl)sulfamoyl]amino]-6-fluoro-phenoxy]quinoxaline (A-83) (190 mg, 224.86 mol, TFA salt) in anhydrous DMAc (5 mL) was added triethylamine (674.58 mol, 94.02 L) at 5° C. and stirred for 30 min. Subsequently, 1-[5-fluoro-1-methyl-6-(6-oxo-2-azaspiro[3.3]heptan-2-yl)indazol-3-yl]hexahydropyrimidine-2,4-dione (B-11) (115.87 mg, 224.86 mol) and AcOH (449.72 mol, 25.72 μL) were added at room temperature. The reaction mixture was stirred at room temperature for 3 hours. Finally, MP-CNBH₃ (190 mg, 224.86 mol) was added and stirred at 70° C. for 16 hours. After completion of the reaction, the reaction mixture was filtered and concentrated under reduced pressure to get the crude compound, which was purified by preparative HPLC [Column: X-Bridge C18 (250 mm*19 mm*5 m), mobile phase A: 10 mM NH₄HCO₃ in milli-Q water; mobile phase B: acetonitrile]. The pure fraction was lyophilized to afford 7-[2-cyano-3-[[ethyl(methyl)sulfamoyl]amino]-6-fluoro-phenoxy]-2-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]quinoxaline (Compound 303, 8.1 mg, 9.26 mol, 4% yield) as an off-white solid. UPLC-MS (ES+): m/z 852.2 [M+H]⁺. ¹H NMR (400 MHz, DMSO-d₆): δ 10.51 (s, 1H), 10.00 (bs, 1H), 8.76 (s, 1H), 8.12 (d, J=9.20 Hz, 1H), 7.68 (dd, J=2.80, 9.20 Hz, 1H), 7.43 (bs, 1H), 7.31-7.25 (m, 2H), 7.12 (d, J=2.80 Hz, 1H), 6.51 (d, J=7.60 Hz, 1H), 4.00-3.79 (m, 13H), 3.01 (q, J=7.20 Hz, 2H), 2.73 (t, J=6.80 Hz, 2H), 2.65-2.59 (m, 7H), 2.27-2.16 (m, 2H), 1.03 (t, J=2.80 Hz, 3H).

### Synthesis D187: Synthesis of 3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-6-[3-[[ethyl(methyl)sulfamoyl]amino]-2,6-difluoro-benzoyl]-4-oxo-quinazoline (Compound 304)

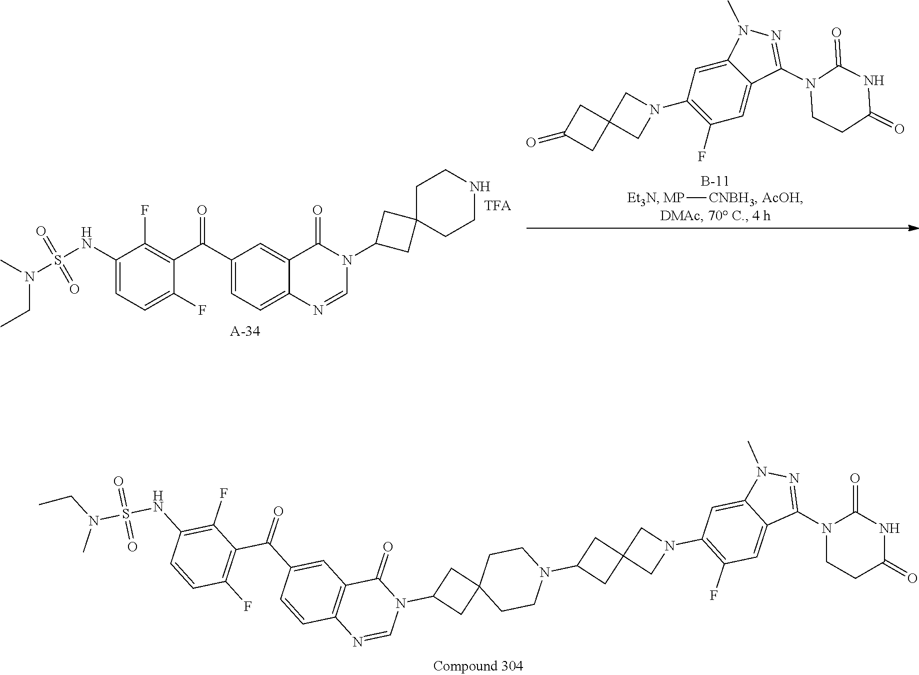

Into a 10 mL glass vial containing a well stirred solution of 3-(7-azaspiro[3.5]nonan-2-yl)-6-[3-[[ethyl(methyl)sulfamoyl]amino]-2,6-difluoro-benzoyl]-4-oxo-quinazoline (A-34) (60 mg, 90.96 mol, TFA salt) in anhydrous DMAc (2.03 mL) was added triethylamine (363.84 mol, 50.71 μL) at 5° C. and stirred for 30 min. Subsequently, 1-[5-fluoro-1-methyl-6-(6-oxo-2-azaspiro[3.3]heptan-2-yl)indazol-3-yl]hexahydropyrimidine-2,4-dione (B-11) (37.16 mg, 100.06 mol) and AcOH (181.92 mol, 10.4 μL) were added at room temperature and stirred at room temperature for 3 hours. Finally, MP-CNBH₃ (60 mg, 90.96 mol) was added and stirred at 70° C. for 4 hours. After completion of the reaction, the reaction mixture was filtered and filtrate was concentrated under reduced pressure to get the crude residue, which was purified by preparative HPLC [Column: X-Bridge C18 (250 mm*19 mm*5 m), Mobile phase A: 10 mM NH₄HCO₃ in milli-Q water; Mobile phase B: acetonitrile]. Collected fractions were lyophilized to afford 3-[7-[2-[3-(2,4-dioxohexahydropyrimidin-1-yl)-5-fluoro-1-methyl-indazol-6-yl]-2-azaspiro[3.3]heptan-6-yl]-7-azaspiro[3.5]nonan-2-yl]-6-[3-[[ethyl(methyl)sulfamoyl]amino]-2,6-difluoro-benzoyl]-4-oxo-quinazoline (Compound 304, 15 mg, 15.95 mol, 18% yield) as an off-white solid. UPLC-MS (ES+): m/z 901.7 [M+H]⁺. ¹H NMR (400 MHz, DMSO-d₆): δ 10.50 (s, 1H), 9.79 (bs, 1H), 8.59 (s, 1H), 8.37 (s, 1H), 8.29 (dd, J=2.00, 8.40 Hz, 1H), 7.87 (d, J=8.40 Hz, 1H), 7.73-7.67 (m, 1H), 7.36 (t, J=8.40 Hz, 1H), 7.26 (d, J=12.40 Hz, 1H), 6.49 (d, J=7.60 Hz, 1H), 4.92-4.88 (m, 1H), 4.00 (s, 2H), 3.89-3.86 (m, 7H), 3.12 (q, J=7.20 Hz, 2H), 2.74-2.67 (m, 5H), 2.34-2.23 (m, 4H), 2.08-2.02 (m, 2H), 1.68-1.53 (m, 4H), 1.02 (t, J=7.20 Hz, 3H).

### Synthesis D188: Synthesis of Synthesis of 6-[2-cyano-3-[[ethyl(methyl)sulfamoyl]amino]-6-fluoro-phenoxy]-3-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]-4-oxo-quinazoline (Compound 305)

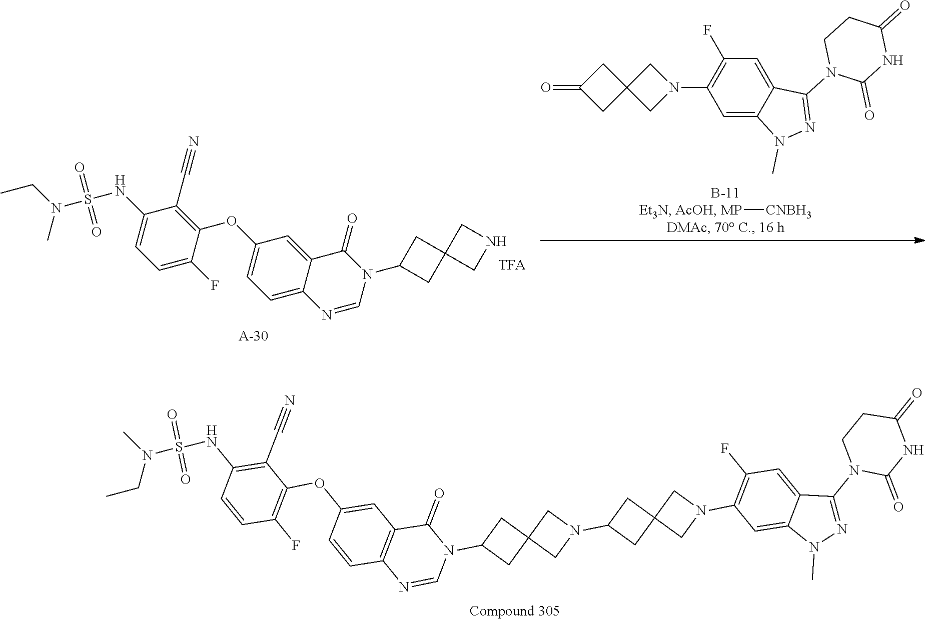

Into a 20 mL pressure vial containing a well-stirred solution of 3-(2-azaspiro[3.3]heptan-6-yl)-6-[2-cyano-3-[[ethyl(methyl)sulfamoyl]amino]-6-fluoro-phenoxy]-4-oxo-quinazoline (A-30) (200 mg, 258.00 mol, TFA salt) in anhydrous DMAc (5 mL) was added triethylamine (774.01 mol, 107.88 μL) at room temperature and the reaction mixture was stirred for 30 min. To the above mixture, 1-[5-fluoro-1-methyl-6-(6-oxo-2-azaspiro[3.3]heptan-2-yl)indazol-3-yl]hexahydropyrimidine-2,4-dione (B-11) (129.83 mg, 258.00 mol) and AcOH (516.01 mol, 29.51 μL) were added at room temperature and mixture was stirred for 5 hours. Subsequently, MP-CNBH₃ (200 mg) was added and resulting mixture was stirred at 70° C. for 16 hours. After completion of the reaction, the reaction mixture was filtered through sintered funnel, washed with dichloromethane and the filtrate was concentrated under reduced pressure to get a crude residue, which was purified by reverse phase by using [column. RediSep R_{f} Gold® reversed-phase C18-teledyne ISCO, 120 g), Mobile phase A: 10 mM NH₄HCO₃ in milli-Q water; Mobile phase B: acetonitrile]. Fractions were lyophilized to afford 6-[2-cyano-3-[[ethyl(methyl)sulfamoyl]amino]-6-fluoro-phenoxy]-3-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]-4-oxo-quinazoline (Compound 305, 16 mg, 17.90 mol, 7% yield) as an off-white solid. UPLC-MS (ES+): m/z 868.4 [M+H]⁺. ¹H NMR (400 MHz, DMSO-d₆): δ 10.51 (s, 1H), 10.02 (bs, 1H), 8.34 (s, 1H), 7.76 (d, J=9.20 Hz, 1H), 7.65 (dd, J=2.80, 8.80 Hz, 1H), 7.43 (bs, 1H), 7.31-7.25 (m, 3H), 6.51 (d, J=7.20 Hz, 1H), 4.93-4.85 (m, 1H), 4.03-3.98 (m, 2H), 3.97-3.92 (m, 2H), 3.91-3.57 (m, 8H), 3.04-2.98 (m, 2H), 2.87-2.71 (m, 6H), 2.64-2.57 (m, 4H), 2.59 (s, 3H), 2.31-2.12 (m, 2H), 1.03 (t, J=7.20 Hz, 3H).

### Synthesis D189: Synthesis of 3-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]-6-[3-[[ethyl(methyl)sulfamoyl]amino]-2,6-difluoro-benzoyl]-4-oxo-quinazoline (Compound 306)

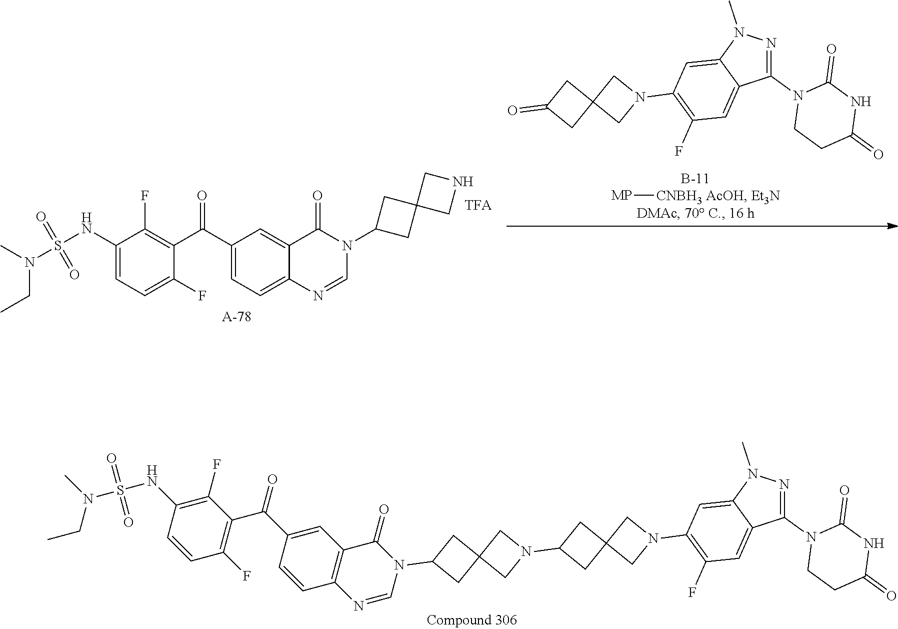

Into a 20 mL glass vial containing a well-stirred solution of 3-(2-azaspiro[3.3]heptan-6-yl)-6-[3-[[ethyl(methyl)sulfamoyl]amino]-2,6-difluoro-benzoyl]-4-oxo-quinazoline (A-78) (200 mg, 286.62 mol, TFA salt) in DMAc (5 mL) was added triethylamine (1.43 mmol, 199.74 μL) and stirred for 10 min at room temperature. Subsequently, 1-[5-fluoro-1-methyl-6-(6-oxo-2-azaspiro[3.3]heptan-2-yl)indazol-3-yl]hexahydropyrimidine-2,4-dione (B-11) (138.97 mg, 315.28 mol) and AcOH (573.24 mol, 32.78 μL) were added and resulting mixture was stirred for 3 h at room temperature. Finally, MP-CNBH₃ (200 mg, 286.62 mol) was added and stirred at 70° C. for 16 hours. After completion of the reaction, the reaction mixture was diluted with dichloromethane (10 mL), filtered through cotton pad and filtrate was concentrated under reduced pressure to get crude residue which was purified by reverse phase [Column: RediSep R_{f} Gold® reversed-phase C18-teledyne ISCO, 120 g, Mobile phase A: 10 mM NH₄HCO₃ in mill-Q water; Mobile phase B: acetonitrile]. Fractions were collected and lyophilized to afford desired product with 93.9% purity, and it was showing aliphatic impurities in NMR. Compound was re-purified by preparative HPLC [Column: X-Bridge C18 (250 mm*19 mm*5 m), Mobile phase A: 10 mM NH₄HCO₃ in milli-Q water; Mobile phase B: acetonitrile]. Fractions were collected and lyophilized to afford 3-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]-6-[3-[[ethyl(methyl)sulfamoyl]amino]-2,6-difluoro-benzoyl]-4-oxo-quinazoline (Compound 306, 10 mg, 11.29 mol, 4% yield) as an off-white solid. UPLC-MS (ES+): m/z 873.3 [M+H]⁺. ¹H NMR (400 MHz, DMSO-d₆): δ 10.50 (s, 1H), 8.56 (s, 1H), 8.37 (d, J=1.60 Hz, 1H), 8.28 (dd, J=2.00, 8.40 Hz, 1H), 7.87 (d, J=8.40 Hz, 1H), 7.72-7.66 (m, 1H), 7.34 (t, J=9.20 Hz, 1H), 7.25 (d, J=12.80 Hz, 1H), 6.50 (d, J=7.60 Hz, 1H), 4.80-4.76 (m, 1H), 3.95-3.86 (m, 9H), 3.25 (s, 3H), 3.14-3.08 (m, 4H), 2.74-2.67 (m, 5H), 2.62-2.56 (m, 4H), 2.24-2.19 (m, 2H), 1.98-1.94 (m, 2H), 1.02 (t, J=7.20 Hz, 3H).

### Synthesis D190: Synthesis of 6-[2-cyano-3-[[ethyl(methyl)sulfamoyl]amino]-6-fluoro-phenoxy]-3-[6-[1-[3-(2,4-dioxohexahydropyrimidin-1-yl)-5-fluoro-1-methyl-indazol-6-yl]-4-piperidyl]-6-azaspiro[3.4]octan-2-yl]-4-oxo-quinazoline (Compound 307 and Compound 308)

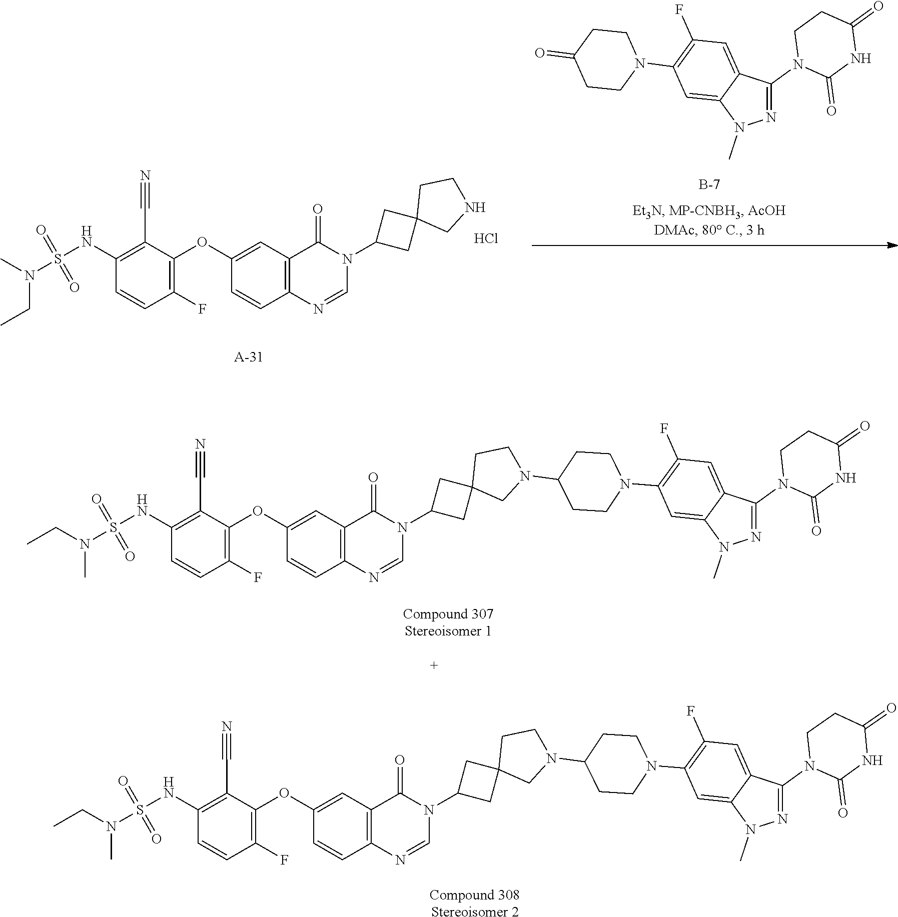

Into a 20 mL glass vial with pressure released cap containing a well-stirred solution of 3-(6-azaspiro[3.4]octan-2-yl)-6-[2-cyano-3-[[ethyl(methyl)sulfamoyl]amino]-6-fluoro-phenoxy]-4-oxo-quinazoline (A-31) (160 mg, 267.03 mol, HCl salt) in anhydrous DMAc (4 mL) was added triethylamine (1.07 mmol, 148.88 μL) and stirred for 10 min, Subsequently, 1-[5-fluoro-1-methyl-6-(4-oxo-1-piperidyl)indazol-3-yl]hexahydropyrimidine-2,4-dione (B-7) (138.89 mg, 293.74 mol) and AcOH (534.07 mol, 30.54 μL) were added and stirred for 2 h at room temperature. Finally, MP-CNBH₃ (160 mg, 267.03 mol) was added and stirred at 80° C. for 3 hours. After completion of the reaction, the reaction mixture was filtered through cotton pad and filtrate was concentrated under reduced pressure to get a crude residue which was purified by reverse phase preparative HPLC [Column: X-BRIDGE C18 (150 mm*19 mm*5 μm), Mobile Phase A: 10 mM NH₄HCO₃ in milli-Q water and NH₄OH (pH 10); Mobile phase B: acetonitrile]. The first eluting fraction (Peak-1 by Prep HPLC sequence and Peak-2 by SFC sequence) was lyophilized to afford:

Peak-1: 6-[2-cyano-3-[[ethyl(methyl)sulfamoyl]amino]-6-fluoro-phenoxy]-3-[6-[1-[3-(2,4-dioxohexahydropyrimidin-1-yl)-5-fluoro-1-methyl-indazol-6-yl]-4-piperidyl]-6-azaspiro[3.4]octan-2-yl]-4-oxo-quinazoline (Compound 307, 17 mg, 19.42 mol, 8% yield) as an off-white solid. Assigned as stereoisomer 1. UPLC-MS (ES+): m/z 870.2 [M+H]⁺. ¹H NMR (400 MHz, DMSO-d₆): δ 10.54 (s, 1H), 9.78 (bs, 1H), 8.39 (s, 1H), 7.76 (d, J=9.20 Hz, 1H), 7.63 (dd, J=3.20, 9.00 Hz, 1H), 7.39-7.22 (m, 4H), 7.14 (d, J=7.20 Hz, 1H), 4.99-4.92 (m, 1H), 3.96 (s, 3H), 3.90 (t, J=6.80 Hz, 2H), 3.53-3.46 (m, 2H), 3.02-2.92 (m, 2H), 2.79-2.63 (m, 5H), 2.58-2.52 (m, 3H), 2.16-2.05 (m, 4H), 1.74-1.65 (m, 2H), 1.03 (t, J=7.20 Hz, 3H).

Similarly, the second eluting fraction (Peak-2 by Prep HPLC sequence and Peak-1 by SFC sequence) was lyophilized to afford Compound 308.

Peak-2: 6-[2-cyano-3-[[ethyl(methyl)sulfamoyl]amino]-6-fluoro-phenoxy]-3-[6-[1-[3-(2,4-dioxohexahydropyrimidin-1-yl)-5-fluoro-1-methyl-indazol-6-yl]-4-piperidyl]-6-azaspiro[3.4]octan-2-yl]-4-oxo-quinazoline (Compound 308, 12 mg, 13.73 mol, 5% yield) as a pale yellow solid. Assigned as stereoisomer 2. UPLC-MS (ES+): m/z 870.3 [M+H]⁺. ¹H NMR (400 MHz, DMSO-d₆): δ 10.54 (s, 1H), 9.78 (bs, 1H), 8.39 (s, 1H), 7.77 (d, J=8.80 Hz, 1H), 7.63 (dd, J=3.20, 8.80 Hz, 1H), 7.39-7.25 (m, 4H), 7.15 (d, J=6.80 Hz, 1H), 4.94-4.90 (m, 1H), 3.96 (s, 3H), 3.90 (t, J=6.40 Hz, 2H), 3.55-3.52 (m, 2H), 3.14-2.97 (m, 2H), 2.76-2.73 (m, 4H), 2.68-2.52 (m, 6H), 2.40-1.98 (m, 4H), 1.78-1.76 (m, 2H), 1.03 (t, J=7.20 Hz, 3H).

### Synthesis D191: Synthesis of (7R)-7-[6-[2-cyano-3-[[ethyl(methyl)sulfamoyl]amino]-6-fluoro-phenoxy]-4-oxo-quinazolin-3-yl]-2-[1-[3-(2,4-dioxohexahydropyrimidin-1-yl)-5-fluoro-1-methyl-indazol-6-yl]-4-piperidyl]-5-oxa-2-azaspiro[3.4]octane (Compound 309)

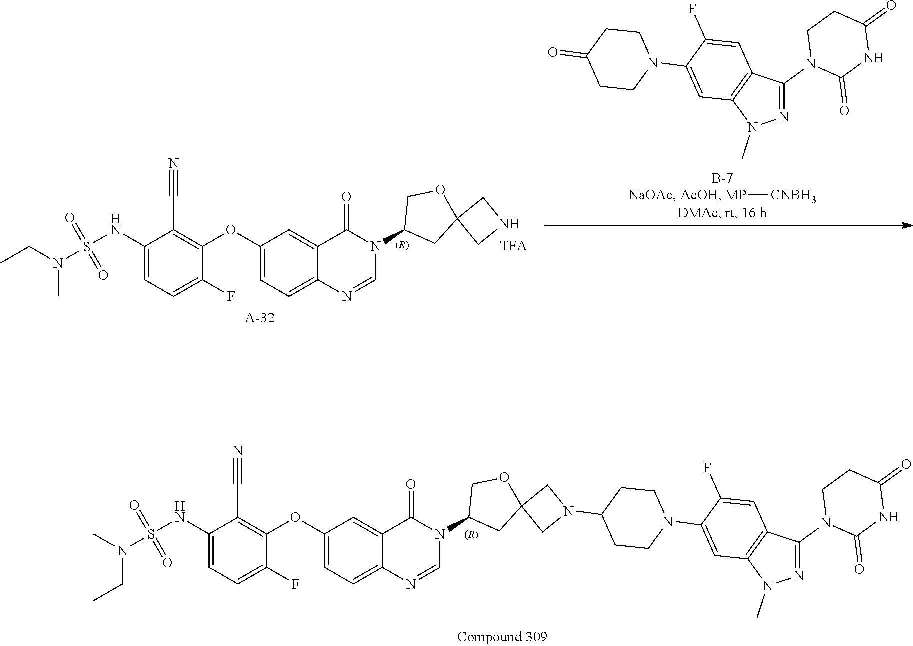

Into a 40 mL pressure vial containing a well-stirred solution of (7R)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-32) (150 mg, 210.09 mol, TFA salt) in anhydrous DMAc (3 mL) was added NaOAc (86.17 mg, 1.05 mmol) at room temperature and resulting mixture was stirred for 1 hour. To the above mixture, 1-[5-fluoro-1-methyl-6-(4-oxo-1-piperidyl)indazol-3-yl]hexahydropyrimidine-2,4-dione (B-7) (82.74 mg, 168.07 mol) and AcOH (630.27 mol, 36.04 L) were added at room temperature and stirred for 5 hours. Subsequently, MP-CNBH₃ (200 mg) was added and resulting reaction mixture was stirred at room temperature for 16 hours. After completion of the reaction, the reaction mixture was filtered through sintered funnel, washed with dichloromethane and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water; Mobile phase B: acetonitrile]. Fractions were collected and lyophilized to afford ((7R)-7-[6-[2-cyano-3-[[ethyl(methyl)sulfamoyl]amino]-6-fluoro-phenoxy]-4-oxo-quinazolin-3-yl]-2-[1-[3-(2,4-dioxohexahydropyrimidin-1-yl)-5-fluoro-1-methyl-indazol-6-yl]-4-piperidyl]-5-oxa-2-azaspiro[3.4]octane, which is not pure. Further repurified by reverse phase column chromatography (Column: Redisep R_{f} Gold® reversed-phase C18-teledyne ISCO, 120 g, Mobile phase A: 10 mM NH₄HCO₃ in milli-Q water; Mobile phase B: acetonitrile). Fractions were collected and lyophilized to afford (7R)-7-[6-[2-cyano-3-[[ethyl(methyl)sulfamoyl]amino]-6-fluoro-phenoxy]-4-oxo-quinazolin-3-yl]-2-[1-[3-(2,4-dioxohexahydropyrimidin-1-yl)-5-fluoro-1-methyl-indazol-6-yl]-4-piperidyl]-5-oxa-2-azaspiro[3.4]octane (Compound 309, 50 mg, 56.76 mol, 27% yield) as an off-white solid. UPLC-MS (ES+): m/z 872.3 [M+H]⁺. ¹H NMR (400 MHz, DMSO-d₆): δ 10.53 (s, 1H), 10.25 (bs, 1H), 8.20 (s, 1H), 7.77 (d, J=9.20 Hz, 1H), 7.69-7.67 (m, 1H), 7.55-7.25 (m, 4H), 7.13 (d, J=6.80 Hz, 1H), 5.28-5.10 (m, 1H), 4.80-4.30 (m, 1H), 4.11 (s, 2H), 3.95 (s, 3H), 3.90 (t, J=6.80 Hz, 2H), 3.48-3.46 (m, 3H), 3.10-2.95 (m, 3H), 2.74 (t, J=6.80 Hz, 4H), 2.64-2.55 (m, 4H), 2.10-1.88 (m, 2H), 1.60-1.40 (m, 2H), 1.03 (t, J=7.20 Hz, 3H).

### Synthesis D192: Synthesis of (7R)-7-[6-[2-cyano-3-[[ethyl(methyl)sulfamoyl]amino]-6-fluoro-phenoxy]-4-oxo-quinazolin-3-yl]-2-[2-[3-(2,4-dioxohexahydropyrimidin-1-yl)-5-fluoro-1-methyl-indazol-6-yl]-2-azaspiro[3.3]heptan-6-yl]-5-oxa-2-azaspiro[3.4]octane (Compound 310)

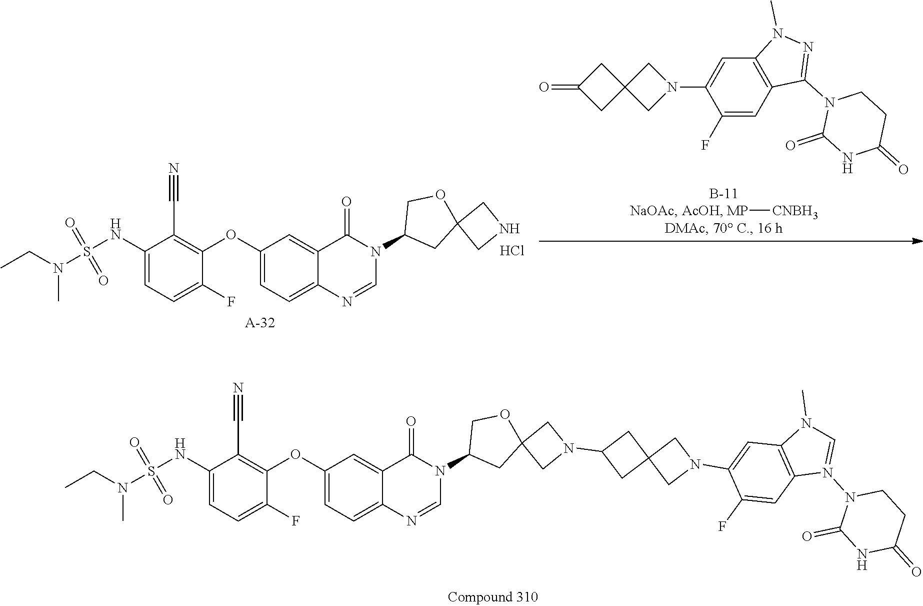

Into a 20 mL pressure vial containing a well-stirred solution of (7R)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-32) (150 mg, 233.43 mol, HCl salt) in anhydrous DMAc (8 mL) was added NaOAc (70.86 mg, 700.30 mol) at room temperature and stirred for 30 min. To the above mixture, 1-[5-fluoro-1-methyl-6-(6-oxo-2-azaspiro[3.3]heptan-2-yl)indazol-3-yl]hexahydropyrimidine-2,4-dione (B-11) (86.69 mg, 233.43 mol) and AcOH (1.17 mmol, 66.75 L) were added at room temperature and stirred for 5 hours. Finally, MP-CNBH₃ (150 mg) was added and resulting mixture was stirred at 70° C. for 16 hours. After completion of the reaction, the reaction mixture was filtered through sintered funnel, washed with dichloromethane and filtrate was concentrated under reduced pressure to get crude residue, which was purified by preparative HPLC [Column: SHIM PACK (150 mm*21.2 mm*5 μm), Mobile Phase A: 10 mM NH₄HCO₃ in milli-Q water; Mobile phase B: acetonitrile]. Collected fractions were lyophilized to afford (7R)-7-[6-[2-cyano-3-[[ethyl(methyl)sulfamoyl]amino]-6-fluoro-phenoxy]-4-oxo-quinazolin-3-yl]-2-[2-[3-(2,4-dioxohexahydropyrimidin-1-yl)-5-fluoro-1-methyl-indazol-6-yl]-2-azaspiro[3.3]heptan-6-yl]-5-oxa-2-azaspiro[3.4]octane (Compound 310, 7 mg, 7.53 mol, 3% yield) as an off-white solid. UPLC-MS (ES+): m/z 884.3 [M+H]⁺. ¹H NMR (400 MHz, DMSO-d₆): δ 10.50 (s, 1H), 10.20 (bs, 1H), 8.19 (s, 1H), 7.77 (d, J=9.20 Hz, 1H), 7.68 (dd, J=3.20, 8.80 Hz, 1H), 7.51 (bs, 1H), 7.33-7.24 (m, 3H), 6.50 (d, J=7.60 Hz, 1H), 5.20-2.12 (m, 1H), 4.10-4.08 (m, 2H), 3.98-3.42 (m, 14H), 3.10-3.03 (m, 2H), 2.74 (t, J=6.40 Hz, 2H), 2.68-2.55 (m, 5H), 2.45-2.35 (m, 2H), 2.25-2.10 (m, 2H), 1.04 (t, J=7.20 Hz, 3H).

### Synthesis D193: Synthesis of (7S)-7-[6-[2-cyano-3-[[ethyl(methyl)sulfamoyl]amino]-6-fluoro-phenoxy]-4-oxo-quinazolin-3-yl]-2-[2-[3-(2,4-dioxohexahydropyrimidin-1-yl)-5-fluoro-1-methyl-indazol-6-yl]-2-azaspiro[3.3]heptan-6-yl]-5-oxa-2-azaspiro[3.4]octane (Compound 311)

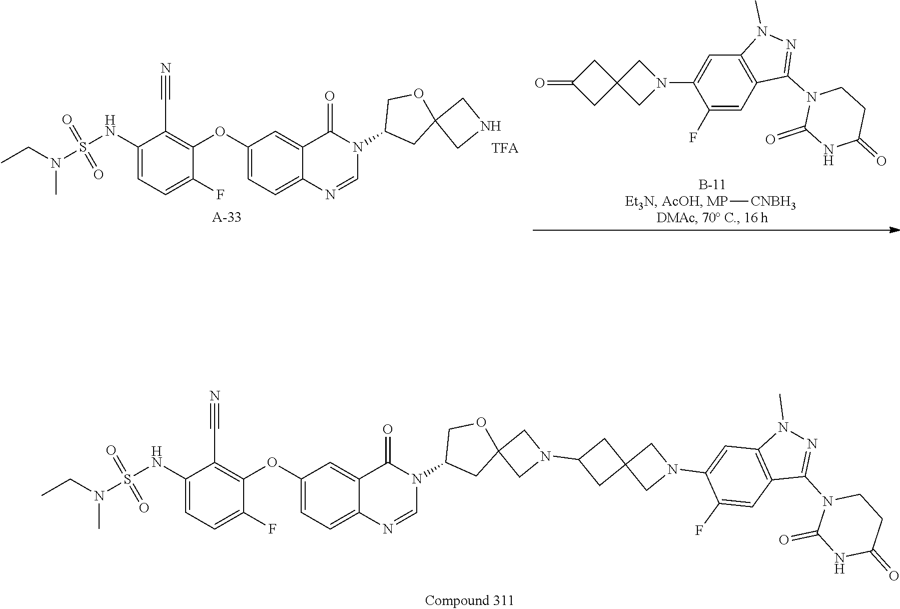

Into a 20 mL pressure vial containing a well-stirred solution of (7S)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-33) (150 mg, 233.43 mol, TFA salt) in anhydrous DMAc (5 mL) was added triethylamine (233.43 mol, 32.54 μL) at room temperature and stirred for 30 min. To the above mixture, 1-[5-fluoro-1-methyl-6-(6-oxo-2-azaspiro[3.3]heptan-2-yl)indazol-3-yl]hexahydropyrimidine-2,4-dione (B-11) (86.69 mg, 233.43 mol) and AcOH (233.43 mol, 13.35 μL) were added at room temperature and stirred for 5 hours. Finally, MP-CNBH₃ (150 mg) was added and stirred at 70° C. for 16 hours. After completion of the reaction, the reaction mixture was filtered through sintered funnel, washed with dichloromethane and filtrate was concentrated under reduced pressure to get a crude residue, which was purified by preparative HPLC [Column: SHIM PACK (150 mm*21.2 mm*5 m), Mobile Phase A: 10 mM NH₄HCO₃ in milli-Q water; Mobile phase B: acetonitrile]. Fractions were collected and lyophilized to afford (7S)-7-[6-[2-cyano-3-[[ethyl(methyl)sulfamoyl]amino]-6-fluoro-phenoxy]-4-oxo-quinazolin-3-yl]-2-[2-[3-(2,4-dioxohexahydropyrimidin-1-yl)-5-fluoro-1-methyl-indazol-6-yl]-2-azaspiro[3.3]heptan-6-yl]-5-oxa-2-azaspiro[3.4]octane (Compound 311, 23 mg, 24.91 mol, 11% yield) as an off-white solid. UPLC-MS (ES+): m/z 884.3 [M+H]⁺. ¹H NMR (400 MHz, DMSO-d₆): δ 10.51 (s, 1H), 10.20 (bs, 1H), 8.19 (s, 1H), 7.78 (d, J=9.20 Hz, 1H), 7.68 (dd, J=2.80, 9.00 Hz, 1H), 7.55 (bs, 1H), 7.33-7.25 (m, 3H), 6.50 (d, J=7.60 Hz, 1H), 5.20-5.16 (m, 1H), 4.12-4.09 (m, 2H), 3.98-3.50 (m, 14H), 3.10-3.03 (m, 2H), 2.75-2.57 (m, 7H), 2.45-2.35 (m, 2H), 2.26-2.12 (m, 2H), 1.04 (t, J=7.20 Hz, 3H).

### Synthesis D194: Synthesis of (7S)-7-[6-[2-cyano-3-[[ethyl(methyl)sulfamoyl]amino]-6-fluoro-phenoxy]-4-oxo-quinazolin-3-yl]-2-[1-[3-(2,4-dioxohexahydropyrimidin-1-yl)-5-fluoro-1-methyl-indazol-6-yl]-4-piperidyl]-5-oxa-2-azaspiro[3.4]octane (Compound 312)

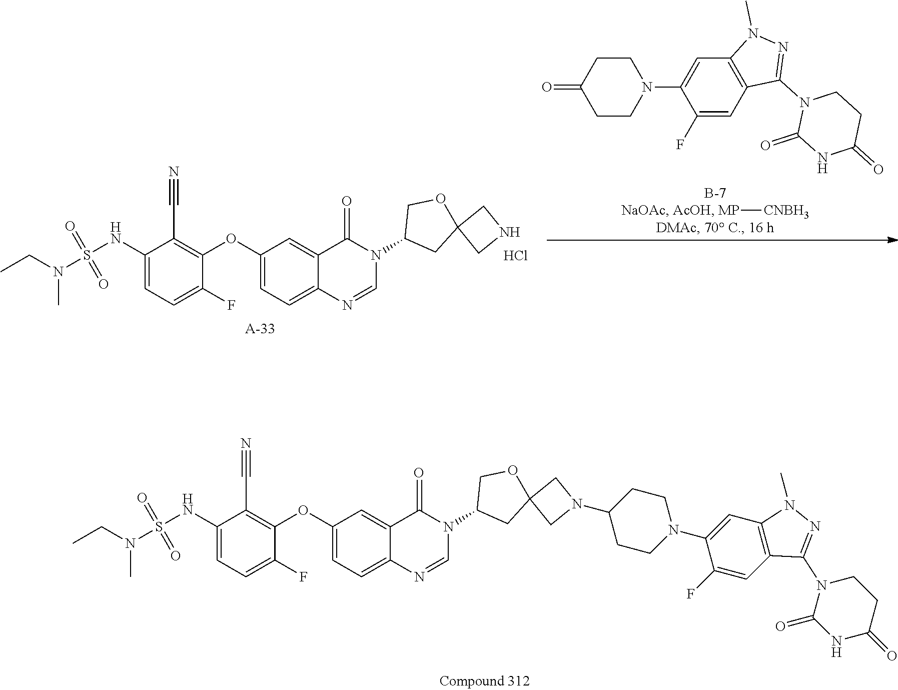

Into a 20 mL pressure vial containing a well-stirred solution of (7S)-7-[6-[2-cyano-3-[[ethyl(methyl)sulfamoyl]amino]-6-fluoro-phenoxy]-4-oxo-quinazolin-3-yl]-5-oxa-2-azaspiro[3.4]octane (A-33) (150 mg, 233.43 mol, HCl salt) in anhydrous DMAc (8 mL) was added NaOAc (95.75 mg, 1.17 mmol) at room temperature and stirred for 30 min. To the above mixture, 1-[5-fluoro-1-methyl-6-(4-oxo-1-piperidyl)indazol-3-yl]hexahydropyrimidine-2,4-dione (B-7) (83.89 mg, 233.43 mol) and AcOH (233.43 mol, 13.35 μL) were added at room temperature and stirred for 5 hours. Finally, MP-CNBH₃ (150 mg) was added and resulting reaction mixture was stirred at 70° C. for 16 hours. After completion of the reaction, the reaction mixture was filtered through sintered funnel, washed with dichloromethane and filtrate was concentrated under reduced pressure to get a crude residue, which was purified by preparative HPLC [Column: X-Bridge C18 (150 mm*19 mm*5 μm), Mobile Phase A: 10 mM NH₄HCO₃ in milli-Q water; Mobile phase B: acetonitrile]. Fractions were collected and lyophilized to afford (7S)-7-[6-[2-cyano-3-[[ethyl(methyl)sulfamoyl]amino]-6-fluoro-phenoxy]-4-oxo-quinazolin-3-yl]-2-[1-[3-(2,4-dioxohexahydropyrimidin-1-yl)-5-fluoro-1-methyl-indazol-6-yl]-4-piperidyl]-5-oxa-2-azaspiro[3.4]octane (Compound 312, 33 mg, 37.47 mol, 16% yield) as an off-white solid. UPLC-MS (ES+): m/z 872.3 [M+H]⁺. ¹H NMR (400 MHz, DMSO-d₆): δ 10.53 (s, 1H), 10.18 (bs, 1H), 8.21 (s, 1H), 7.78 (d, J=8.80 Hz, 1H), 7.69 (dd, J=3.20, 9.00 Hz, 1H), 7.51 (bs, 1H), 7.37-7.32 (m, 3H), 7.13 (d, J=7.20 Hz, 1H), 5.20-5.16 (m, 1H), 4.75-3.70 (m, 11H), 3.50-3.42 (m, 2H), 3.10-3.00 (m, 3H), 2.77-2.60 (m, 9H), 2.06-1.88 (m, 2H), 1.55-1.42 (m, 2H), 1.04 (t, J=7.20 Hz, 3H).

### Synthesis D195: Synthesis of (6-[2-cyano-3-[[ethyl(methyl)sulfamoyl]amino]-6-fluoro-phenoxy]-3-[2-[[1-[3-(2,4-dioxohexahydropyrimidin-1-yl)-5-fluoro-1-methyl-indazol-6-yl]azetidin-3-yl]methyl]-2-azaspiro[3.3]heptan-6-yl]-4-oxo-quinazoline (Compound 313)

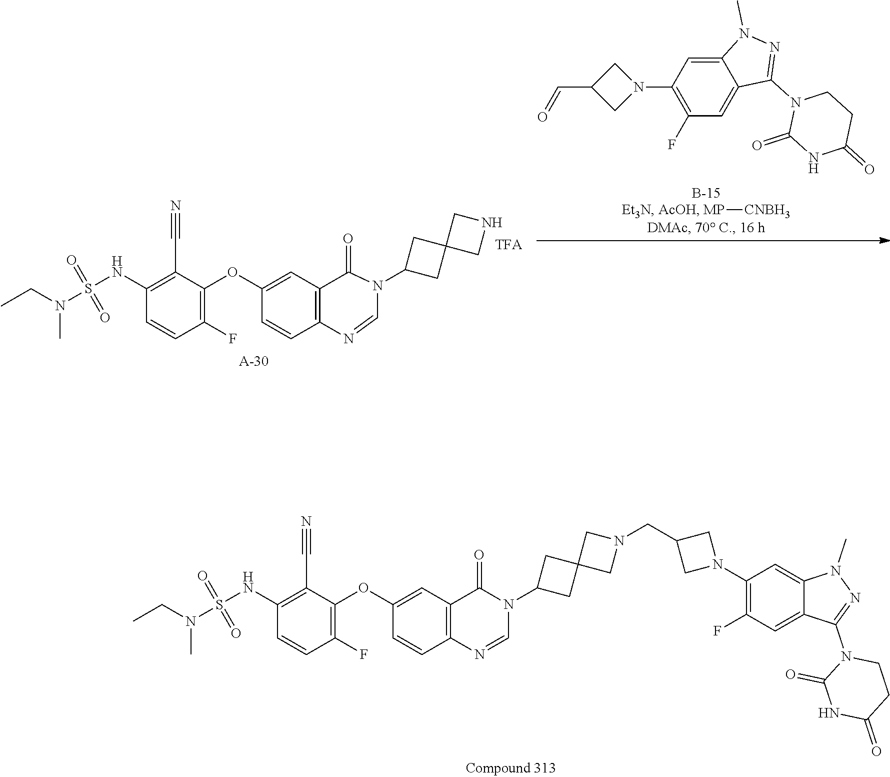

Into a 20 mL pressure vial containing a well-stirred solution of 3-(2-azaspiro[3.3]heptan-6-yl)-6-[2-cyano-3-[[ethyl(methyl)sulfamoyl]amino]-6-fluoro-phenoxy]-4-oxo-quinazoline (A-30) (100 mg, 159.60 mol, TFA salt) in anhydrous DMAc (3.01 mL) was added triethylamine (478.79 mol, 66.73 μL) at room temperature and resulting mixture was stirred for 30 min. To the above mixture, 1-[3-(2,4-dioxohexahydropyrimidin-1-yl)-5-fluoro-1-methyl-indazol-6-yl]azetidine-3-carbaldehyde (B-15) (55.11 mg, 159.60 mol) and AcOH (319.19 mol, 18.25 μL) were added and stirred for 5 hours. Finally, MP-CNBH₃ (100 mg, 159.60 mol) was added, and reaction mixture was stirred at 70° C. for 16 hours. After completion of the reaction, the reaction mixture was filtered through sintered funnel, washed with dichloromethane and the filtrate was concentrated under reduced pressure to get a crude residue which was purified by preparative HPLC [Column: X-Bridge C18 (150 mm*19.5 mm*5 m), Mobile phase A: 10 mM NH₄HCO₃ in milli-Q water; Mobile phase B: acetonitrile] to afford (6-[2-cyano-3-[[ethyl(methyl)sulfamoyl]amino]-6-fluoro-phenoxy]-3-[2-[[1-[3-(2,4-dioxohexahydropyrimidin-1-yl)-5-fluoro-1-methyl-indazol-6-yl]azetidin-3-yl]methyl]-2-azaspiro[3.3]heptan-6-yl]-4-oxo-quinazoline (Compound 313, 17 mg, 20.03 mol, 13% yield) as an off-white solid. UPLC-MS (ES+): m/z 842.3 [M+H]⁺. ¹H NMR (400 MHz, DMSO-d₆): δ 10.51 (s, 1H), 9.86 (bs, 1H), 8.35 (s, 1H), 7.76 (d, J=8.80 Hz, 1H), 7.64 (dd, J=3.20, 9.00 Hz, 1H), 7.41 (bs, 1H), 7.31-7.26 (m, 3H), 6.53 (d, J=7.60 Hz, 1H), 4.88-4.38 (m, 1H), 4.10-3.85 (m, 11H), 3.75-3.68 (m, 2H), 3.28-3.22 (m, 2H), 3.09-2.98 (m, 2H), 2.93-2.70 (m, 7H), 2.59 (s, 3H), 1.03 (t, J=7.20 Hz, 3H).

### Synthesis D196: Synthesis of 6-[2-cyano-3-[[ethyl(methyl)sulfamoyl]amino]-6-fluoro-phenoxy]-3-[2-[7-[3-(2,4-dioxohexahydropyrimidin-1-yl)-5-fluoro-1-methyl-indazol-6-yl]-7-azaspiro[3.5]nonan-2-yl]-2-azaspiro[3.3]heptan-6-yl]-4-oxo-quinazoline (Compound 314)

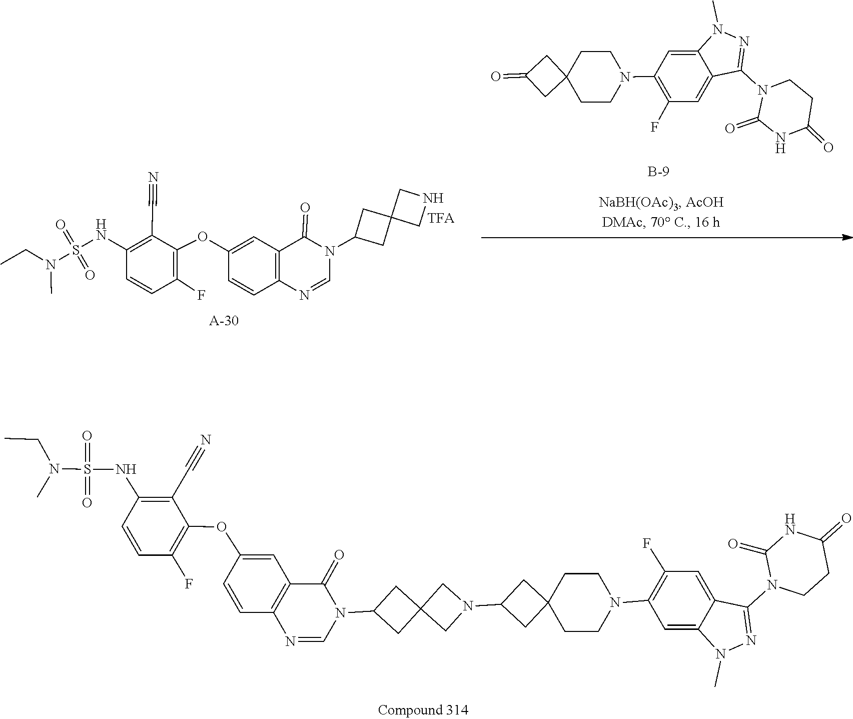

Into a 20 mL pressure vial containing a well-stirred solution of 3-(2-azaspiro[3.3]heptan-6-yl)-6-[2-cyano-3-[[ethyl(methyl)sulfamoyl]amino]-6-fluoro-phenoxy]-4-oxo-quinazoline (A-30) (166.91 mg, 215.31 mol, TFA salt) and 1-[5-fluoro-1-methyl-6-(2-oxo-7-azaspiro[3.5]nonan-7-yl)indazol-3-yl]hexahydropyrimidine-2,4-dione (B-9) (86 mg, 215.31 mol) in DMAc (6 mL) were added sodium-triacetoxyborohydride (228.17 mg, 1.08 mmol) and AcOH (215.31 mol, 12.31 μL) at 0° C. under nitrogen atmosphere. Resulting reaction mixture was stirred at 70° C. for 16 hours. After completion of the reaction, the reaction mixture was filtered through sintered funnel, washed with dichloromethane and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water; Mobile phase B: acetonitrile]. The eluting fractions were lyophilized to afford 6-[2-cyano-3-[[ethyl(methyl)sulfamoyl]amino]-6-fluoro-phenoxy]-3-[2-[7-[3-(2,4-dioxohexahydropyrimidin-1-yl)-5-fluoro-1-methyl-indazol-6-yl]-7-azaspiro[3.5]nonan-2-yl]-2-azaspiro[3.3]heptan-6-yl]-4-oxo-quinazoline (Compound 314, 36.88 mg, 40.29 mol, 19% yield) as an off-white solid. UPLC-MS (ES+): m/z 896.4 [M+H]⁺. ¹H NMR (400 MHz, DMSO-d₆): δ 10.53 (s, 1H), 9.97 (bs, 1H), 8.34 (s, 1H), 7.61 (d, J=9.2 Hz, 1H), 7.64 (dd, J=2.80, 9.00 Hz, 1H), 7.51-7.19 (m, 4H), 7.10 (d, J=7.2 Hz, 1H), 4.87-4.78 (m, 1H), 4.42-3.77 (m, 10H), 3.09-2.96 (m, 6H), 2.89-2.71 (m, 6H), 2.67 (m, 3H), 2.25-2.05 (m, 2H), 1.91-1.65 (m, 6H), 1.03 (t, J=7.2 Hz, 3H).

### Synthesis D197: Synthesis of 6-[2-cyano-3-[[ethyl(methyl)sulfamoyl]amino]-6-fluoro-phenoxy]-3-[7-[2-[1-[3-(2,4-dioxohexahydropyrimidin-1-yl)-5-fluoro-1-methyl-indazol-6-yl]-4-hydroxy-4-piperidyl]acetyl]-7-azaspiro[3.5]nonan-2-yl]-4-oxo-quinazoline (Compound 315)

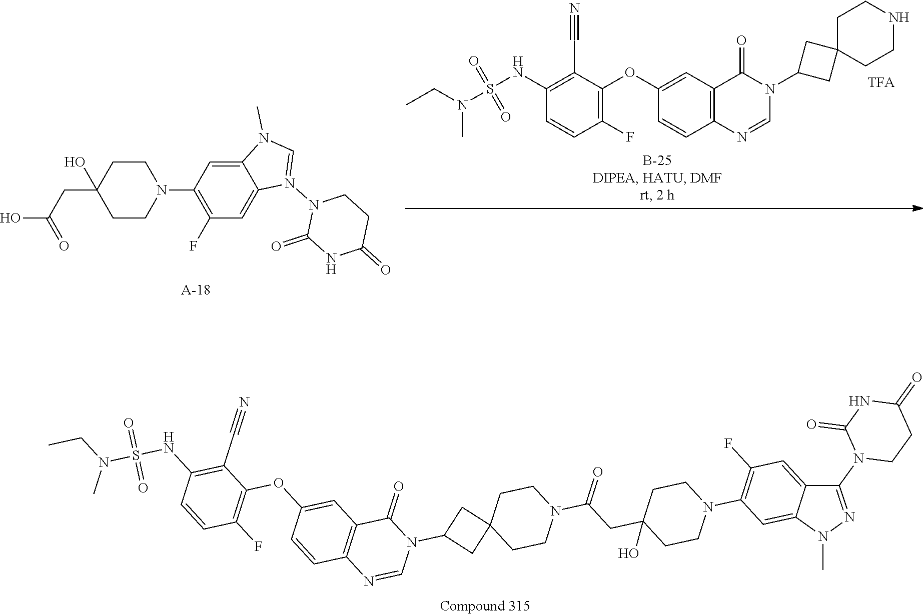

Into a 10 mL single-neck round-bottom flask containing a well-stirred solution of 2-[1-[3-(2,4-dioxohexahydropyrimidin-1-yl)-5-fluoro-1-methyl-indazol-6-yl]-4-hydroxy-4-piperidyl]acetic acid (A-18) (100 mg, 110.79 mol, TFA salt) and 3-(7-azaspiro[3.5]nonan-2-yl)-6-[2-cyano-3-[[ethyl(methyl)sulfamoyl]amino]-6-fluoro-phenoxy]-4-oxo-quinazoline (B-25) (72.53 mg, 110.79 mol, TFA salt) in N,N-dimethylformamide (3 mL) was added N,N-diisopropylethylamine (553.96 mol, 96.49 μL) at room temperature. Subsequently, HATU (84.25 mg, 221.59 mol) was added at room temperature and resulting reaction mixture was stirred at the same temperature for 2 hours. After completion of the reaction, the reaction mixture was concentrated under reduced pressure to get crude residue which was purified by preparative HPLC (Column: X Bridge C18 (250 mm*19 mm*5 m), Mobile phase A: 10 mM NH₄HCO₃ and NH₄OH (pH-10); Mobile phase B: acetonitrile]. Fractions were concentrated under reduced pressure at 40° C. and lyophilized to afford 6-[2-cyano-3-[[ethyl(methyl)sulfamoyl]amino]-6-fluoro-phenoxy]-3-[7-[2-[1-[3-(2,4-dioxohexahydropyrimidin-1-yl)-5-fluoro-1-methyl-indazol-6-yl]-4-hydroxy-4-piperidyl]acetyl]-7-azaspiro[3.5]nonan-2-yl]-4-oxo-quinazoline (Compound 315, 24 mg, 25.39 mol, 23% yield) as an off-white solid. UPLC-MS (ES+): m/z 942.3 [M+H]⁺. ¹H NMR (400 MHz, DMSO-d₆): δ 10.52 (s, 1H), 10.18 (bs, 1H), 8.42 (d, J=3.60 Hz, 1H), 7.80-7.66 (m, 3H), 7.48-7.44 (m, 1H), 7.36-7.30 (m, 2H), 7.13 (d, J=7.20 Hz, 1H), 5.06-5.04 (m, 1H), 5.01-4.91 (m, 1H), 3.95 (s, 3H), 3.89 (t, J=6.40 Hz, 2H), 3.58-3.52 (m, 2H), 3.47-3.41 (m, 2H), 3.20-3.01 (m, 6H), 2.80-2.72 (m, 5H), 2.58-2.55 (m, 2H), 2.41-2.27 (m, 4H), 1.88-1.76 (m, 2H), 1.75-1.55 (m, 6H), 1.05 (t, J=7.20 Hz, 3H).

### Synthesis D198: Synthesis of 6-[2-cyano-3-[[ethyl(methyl)sulfamoyl]amino]-6-fluoro-phenoxy]-3-[2-[1-[3-(2,4-dioxohexahydropyrimidin-1-yl)-5-fluoro-1-methyl-indazol-6-yl]-4-piperidyl]-2-azaspiro[3.3]heptan-6-yl]-4-oxo-quinazoline (Compound 316)

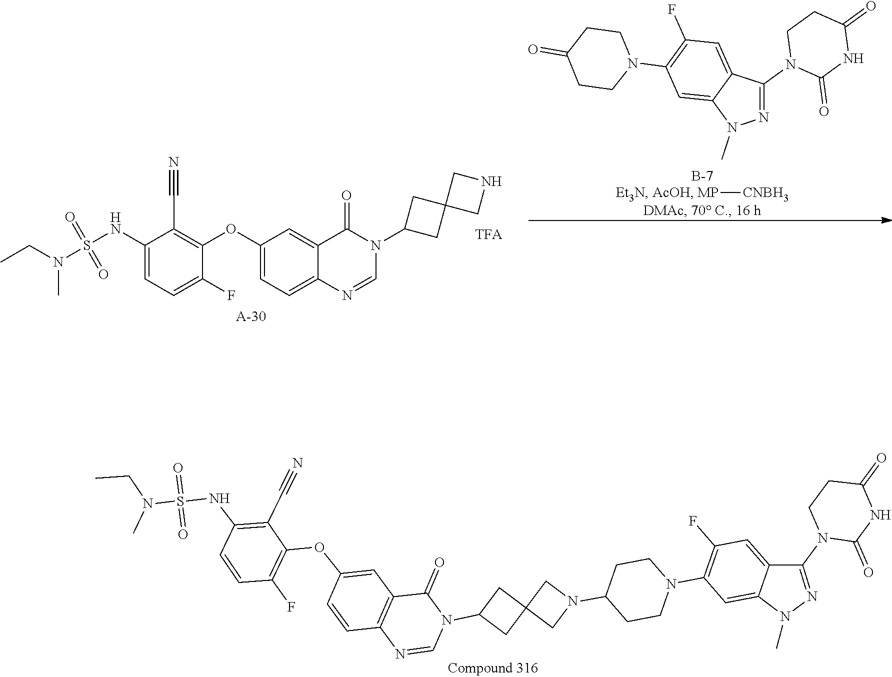

Into a 20 mL pressure vial containing a well-stirred solution of 3-(2-azaspiro[3.3]heptan-6-yl)-6-[2-cyano-3-[[ethyl(methyl)sulfamoyl]amino]-6-fluoro-phenoxy]-4-oxo-quinazoline (A-30) (200 mg, 258.00 mol, TFA salt) in anhydrous DMAc (5 mL) was added triethylamine (774.01 mol, 107.88 μL) at room temperature and the reaction mixture was stirred for 30 min. To the above mixture, 1-[5-fluoro-1-methyl-6-(4-oxo-1-piperidyl)indazol-3-yl]hexahydropyrimidine-2,4-dione (B-7) (119.98 mg, 283.80 mol) and AcOH (516.01 mol, 29.51 μL) were added at room temperature and mixture was stirred for 5 hours. Subsequently, MP-CNBH₃ (150 mg) was added and resulting mixture was stirred at 70° C. for 16 hours. After completion of the reaction, the reaction mixture was filtered through sintered funnel, washed with dichloromethane and the filtrate was concentrated under reduced pressure to get a crude residue, which was purified by reverse phase by using [colum: RediSep R_{f} Gold® reversed-phase C18-teledyne ISCO, 120 g] and [Mobile phase A: 10 mM NH₄HCO₃ in mill-Q water; Mobile phase B: acetonitrile]. Fractions were lyophilized to afford 6-[2-cyano-3-[[ethyl(methyl)sulfamoyl]amino]-6-fluoro-phenoxy]-3-[2-[1-[3-(2,4-dioxohexahydropyrimidin-1-yl)-5-fluoro-1-methyl-indazol-6-yl]-4-piperidyl]-2-azaspiro[3.3]heptan-6-yl]-4-oxo-quinazoline, which is not pure. Further repurified by preparative HPLC [Column: X-Bridge C18 (250 mm*19 mm*5 m), Mobile phase A: 10 mM NH₄HCO₃ in milli-Q water; Mobile phase B: acetonitrile]. Pure fractions were lyophilized to afford 6-[2-cyano-3-[[ethyl(methyl)sulfamoyl]amino]-6-fluoro-phenoxy]-3-[2-[1-[3-(2,4-dioxohexahydropyrimidin-1-yl)-5-fluoro-1-methyl-indazol-6-yl]-4-piperidyl]-2-azaspiro[3.3]heptan-6-yl]-4-oxo-quinazoline (Compound 316, 12 mg, 13.99 mol, 5% yield) as an off-white solid. UPLC-MS (ESI): m/z 856.3 [M+H]⁺. ¹H NMR (400 MHz, DMSO-d₆): δ 10.53 (s, 1H), 9.96 (s, 1H), 8.35 (s, 1H), 7.76 (d, J=8.80 Hz, 1H), 7.65 (dd, J=3.20, 9.00 Hz, 1H), 7.37 (d, J=12.80 Hz, 2H), 7.31 (d, J=2.80 Hz, 1H), 7.28 (s, 1H), 7.14 (d, J=6.80 Hz, 1H), 4.89-4.80 (m, 1H), 4.30-4.12 (m, 2H), 3.96 (s, 3H), 3.90 (t, J=6.80 Hz, 2H), 3.47-3.41 (m, 3H), 3.03-2.98 (m, 3H), 2.76-2.67 (m, 8H), 2.59-2.50 (m, 4H), 2.05-1.87 (m, 2H), 1.49-1.47 (m, 2H), 1.03 (t, J=7.20 Hz, 3H).

### Synthesis D199: Synthesis of 3-[2-[4-[3-(2,4-dioxohexahydropyrimidin-1-yl)-5-fluoro-1-methyl-indazol-6-yl]cyclohexyl]-2-azaspiro[3.3]heptan-6-yl]-6-[3-[[ethyl(methyl)sulfamoyl]amino]-2,6-difluoro-benzoyl]-4-oxo-quinazoline (Compound 317 and Compound 318)

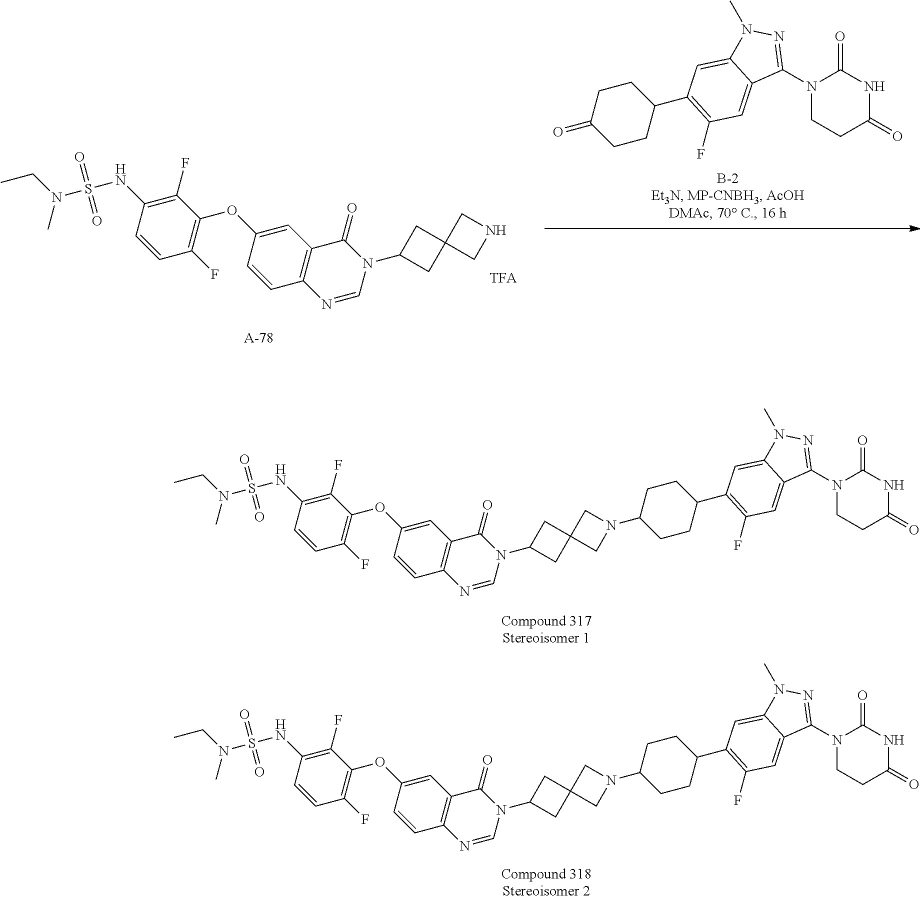

Into a 20 mL pressure vial containing a well-stirred solution of 3-(2-azaspiro[3.3]heptan-6-yl)-6-[3-[[ethyl(methyl)sulfamoyl]amino]-2,6-difluoro-benzoyl]-4-oxo-quinazoline (A-78) (200 mg, 286.62 mol, TFA salt) in DMAc (5 mL) was added triethylamine (1.43 mmol, 199.74 L) at room temperature and stirred for 10 min. Subsequently, 1-[5-fluoro-1-methyl-6-(4-oxocyclohexyl)indazol-3-yl]hexahydropyrimidine-2,4-dione (B-2) (132.92 mg, 315.28 mol) and AcOH (573.24 mol, 32.78 μL) were added and stirred for 3 h at room temperature. Finally, MP-CNBH₃ (200 mg, 286.62 mol) was added and resulting reaction mixture was stirred at 70° C. for 16 hours. After completion of the reaction, the reaction mixture was filtered through cotton pad and filtrate was concentrated under reduced pressure to get a crude residue which was purified by preparative HPLC [Column: X-Bridge C18 (150 mm*19 mm*5 m), Mobile phase A: 10 mM NH₄HCO₃ in milli-Q water; Mobile phase B: acetonitrile]. The first eluting fraction (Peak-1) was lyophilized to afford Compound 317 (assigned as stereoisomer 1).

Peak-1: 3-[2-[4-[3-(2,4-dioxohexahydropyrimidin-1-yl)-5-fluoro-1-methyl-indazol-6-yl]cyclohexyl]-2-azaspiro[3.3]heptan-6-yl]-6-[3-[[ethyl(methyl)sulfamoyl]amino]-2,6-difluoro-benzoyl]-4-oxo-quinazoline (Compound 317, 16 mg, 18.45 mol, 6% yield) as an off-white solid. UPLC-MS (ES+): m/z 860.3 [M+H]⁺. ¹H NMR (400 MHz, DMSO-d₆): δ 10.55 (s, 1H), 8.57 (s, 1H), 8.37 (s, 1H), 8.29 (dd, J=2.00, 8.80 Hz, 1H), 7.87 (d, J=8.80 Hz, 1H), 7.69-7.67 (m, 1H), 7.54 (d, J=6.00 Hz, 1H), 7.37-7.32 (m, 2H), 4.85-4.75 (m, 1H), 3.99 (s, 3H), 3.90 (t, J=6.80 Hz, 2H), 3.21-3.16 (m, 2H), 3.13-3.08 (m, 2H), 2.77-2.72 (m, 7H), 2.65-2.58 (m, 4H), 2.14-2.02 (m, 1H), 1.91-1.86 (m, 4H), 1.63-1.54 (m, 2H), 1.15-1.07 (m, 2H), 1.02 (t, J=7.20 Hz, 3H).

Similarly, second eluting fraction (Peak-2) was lyophilized to afford Compound 318 (assigned as stereoisomer 2). Peak-2: 3-[2-[4-[3-(2,4-dioxohexahydropyrimidin-1-yl)-5-fluoro-1-methyl-indazol-6-yl]cyclohexyl]-2-azaspiro[3.3]heptan-6-yl]-6-[3-[[ethyl(methyl)sulfamoyl]amino]-2,6-difluoro-benzoyl]-4-oxo-quinazoline (Compound 318, 6.0 mg, 6.92 mol, 2% yield) as an off-white solid. UPLC-MS (ES+): m/z 860.3 [M+H]⁺. ¹H NMR (400 MHz, DMSO-d₆): δ 10.55 (s, 1H), 8.58 (s, 1H), 8.37 (s, 1H), 8.28 (dd, J=2.00, 8.60 Hz, 1H), 7.87 (d, J=8.80 Hz, 1H), 7.69-7.67 (m, 1H), 7.47 (d, J=5.60 Hz, 1H), 7.36-7.30 (m, 2H), 4.83-4.78 (m, 1H), 4.02 (s, 3H), 3.90 (t, J=6.80 Hz, 2H), 3.20-3.16 (m, 2H), 3.16-3.08 (m, 3H), 2.89-2.79 (m, 2H), 2.77-2.72 (m, 2H), 2.71 (s, 3H), 2.65-2.58 (m, 4H), 2.38-2.35 (m, 1H), 1.91-1.81 (m, 2H), 1.75-1.70 (m, 2H), 1.60-1.45 (m, 4H), 1.02 (t, J=7.20 Hz, 3H).

### Synthesis D200: Synthesis of 7-[2-cyano-6-fluoro-3-[[2-hydroxyethyl(methyl)sulfamoyl]amino]phenoxy]-2-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]quinoxaline (Compound 319)

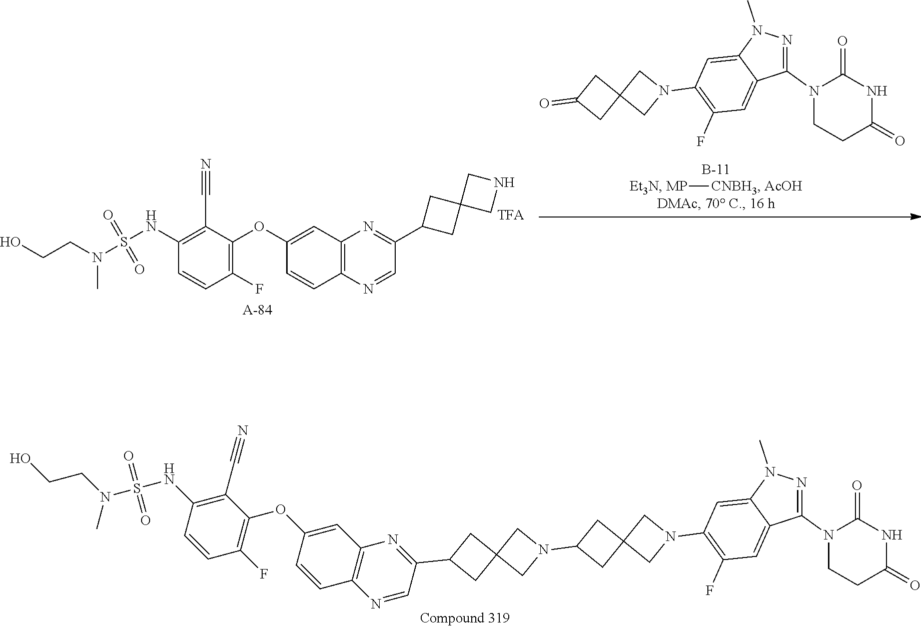

Into a 30 mL glass vial containing a well stirred solution of 2-(2-azaspiro[3.3]heptan-6-yl)-7-[2-cyano-6-fluoro-3-[[2-hydroxyethyl(methyl)sulfamoyl]amino]phenoxy]quinoxaline (A-84) (150 mg, 174.28 mol, TFA salt) in anhydrous DMAc (4 mL) was added triethylamine (522.84 mol, 72.87 μL) at 5° C. and stirred at room temperature for 30 min. Subsequently, 1-[5-fluoro-1-methyl-6-(6-oxo-2-azaspiro[3.3]heptan-2-yl)indazol-3-yl]hexahydropyrimidine-2,4-dione (B-11, 76.14 mg, 174.28 mol) and AcOH (348.56 mol, 19.93 μL) were added at room temperature. The reaction mixture was stirred at room temperature for 3 hours. Finally, MP-CNBH₃ (300 mg, 174.28 mol) was added and stirred at 70° C. for 16 hours. After completion of the reaction, the reaction mixture was filtered, and the filtrate was concentrated under reduced pressure to get the crude residue which was purified by preparative HPLC [Column: X-Bridge C18 (250 mm*19 mm*5 m), mobile phase A: 10 mM NH₄HCO₃ in milli-Q water; Mobile phase B: acetonitrile]. The pure fractions were lyophilized to afford 7-[2-cyano-6-fluoro-3-[[2-hydroxyethyl(methyl)sulfamoyl]amino]phenoxy]-2-[2-[2-[3-(2,4-dioxohexahydropyrimidin-1-yl)-5-fluoro-1-methyl-indazol-6-yl]-2-azaspiro[3.3]heptan-6-yl]-2-azaspiro[3.3]heptan-6-yl]quinoxaline (Compound 319, 13 mg, 14.43 mol, 8% yield) as an off-white solid. UPLC-MS (ES+): m/z 868.2 [M+H]⁺. ¹H NMR (400 MHz, DMSO-d₆): δ 10.51 (s, 1H), 9.93 (bs, 1H), 8.76 (s, 1H), 8.12 (d, J=8.80 Hz, 1H), 7.68 (dd, J=2.80, 9.20 Hz, 1H), 7.50-7.22 (m, 3H), 7.13 (d, J=2.40 Hz, 1H), 6.51 (d, J=7.20 Hz, 1H), 4.20-3.70 (m, 15H), 3.50 (t, J=6.40 Hz, 2H), 3.05 (m, 2H), 2.73 (t, J=6.80 Hz, 2H), 2.70-2.59 (m, 7H), 2.45-2.40 (m, 2H), 2.26-2.18 (m, 2H).

### Synthesis D201: Synthesis of N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-2-oxa-6-azaspiro[3.3]heptane-6-sulfonamide (Compound 320)

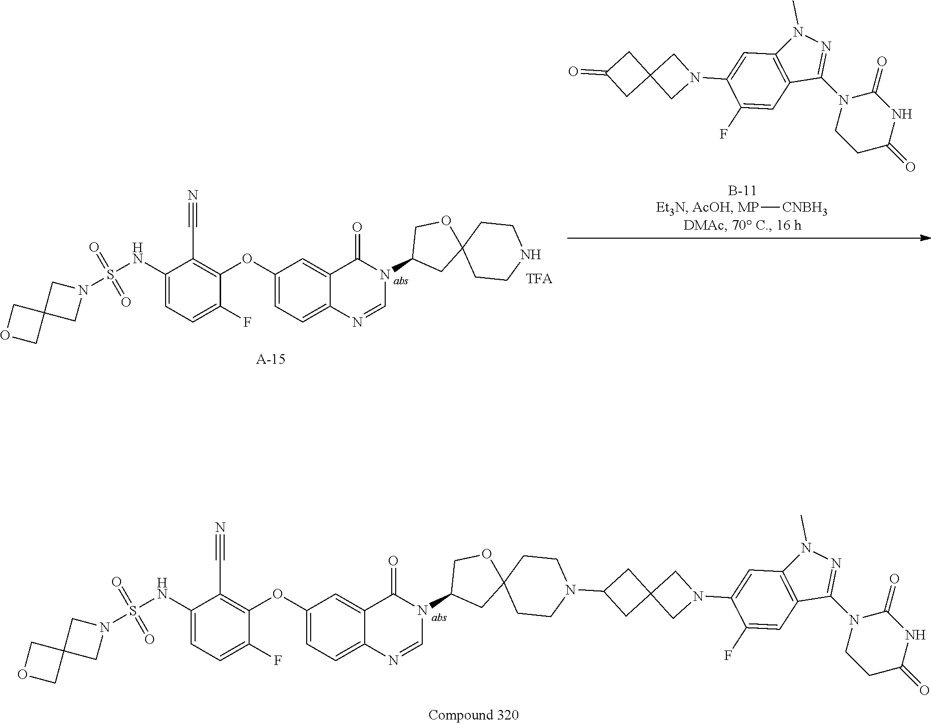

Into a 30 mL glass sealed vial containing a well stirred solution of N-[2-cyano-4-fluoro-3-[3-[(3R)-1-oxa-8-azaspiro[4.5]decan-3-yl]-4-oxo-quinazolin-6-yl]oxy-phenyl]-2-oxa-6-azaspiro[3.3]heptane-6-sulfonamide (A-15) (150 mg, 165.55 mol, TFA salt) in DMAc (3 mL) was added triethylamine (496.63 mol, 69.22 μL) at 5° C. and stirred at room temperature for 30 min. Subsequently, 1-[5-fluoro-1-methyl-6-(6-oxo-2-azaspiro[3.3]heptan-2-yl)indazol-3-yl]hexahydropyrimidine-2,4-dione (B-11) (72.33 mg, 165.55 mol) and AcOH (331.09 mol, 18.93 μL) were added at room temperature. The reaction mixture was stirred at room temperature for 3 hours. Finally, MP-CNBH₃ (150 mg, 165.55 mol) was added and stirred at 70° C. for 16 hours. After completion of the reaction, the reaction mixture was filtered and the filtrate was concentrated under reduced pressure to get a crude residue, which was purified by preparative HPLC [Column: X-Bridge C18 (250 mm*19 mm*5 m), mobile phase A: 10 mM NH₄HCO₃ in milli-Q water; mobile phase B: acetonitrile]. The pure fractions were lyophilized to afford N-[2-cyano-3-[3-[(3R)-8-[2-[3-(2,4-dioxohexahydropyrimidin-1-yl)-5-fluoro-1-methyl-indazol-6-yl]-2-azaspiro[3.3]heptan-6-yl]-1-oxa-8-azaspiro[4.5]decan-3-yl]-4-oxo-quinazolin-6-yl]oxy-4-fluoro-phenyl]-2-oxa-6-azaspiro[3.3]heptane-6-sulfonamide (Compound 320, 17 mg, 16.89 mol, 10% yield) as an off-white solid. UPLC-MS (ES+): m/z 952.2 [M+H]⁺. ¹H NMR (400 MHz, DMSO-d₆): δ 10.51 (s, 1H), 9.24 (bs, 1H), 8.31 (s, 1H), 7.76 (d, J=9.20 Hz, 1H), 7.64 (dd, J=2.80, 9.00 Hz, 1H), 7.36-7.26 (m, 4H), 6.53 (d, J=7.20 Hz, 1H), 5.34-5.25 (m, 1H), 4.61 (s, 4H), 4.22-4.05 (m, 2H), 4.03 (s, 2H), 3.98-3.83 (m, 7H), 3.78 (s, 4H), 3.10-2.76 (m, 2H), 2.73 (t, J=6.80 Hz, 2H), 2.42-2.31 (m, 3H), 2.25-1.95 (m, 4H), 1.95-1.50 (m, 4H).

### Synthesis D202: Synthesis of 7-[2-cyano-6-fluoro-3-[[2-hydroxyethyl(methyl)sulfamoyl]amino]phenoxy]-2-[2-[4-[3-(2,4-dioxohexahydropyrimidin-1-yl)-5-fluoro-1-methyl-indazol-6-yl]cyclohexyl]-2-azaspiro[3.3]heptan-6-yl]quinoxaline (Compound 321 and Compound 322)

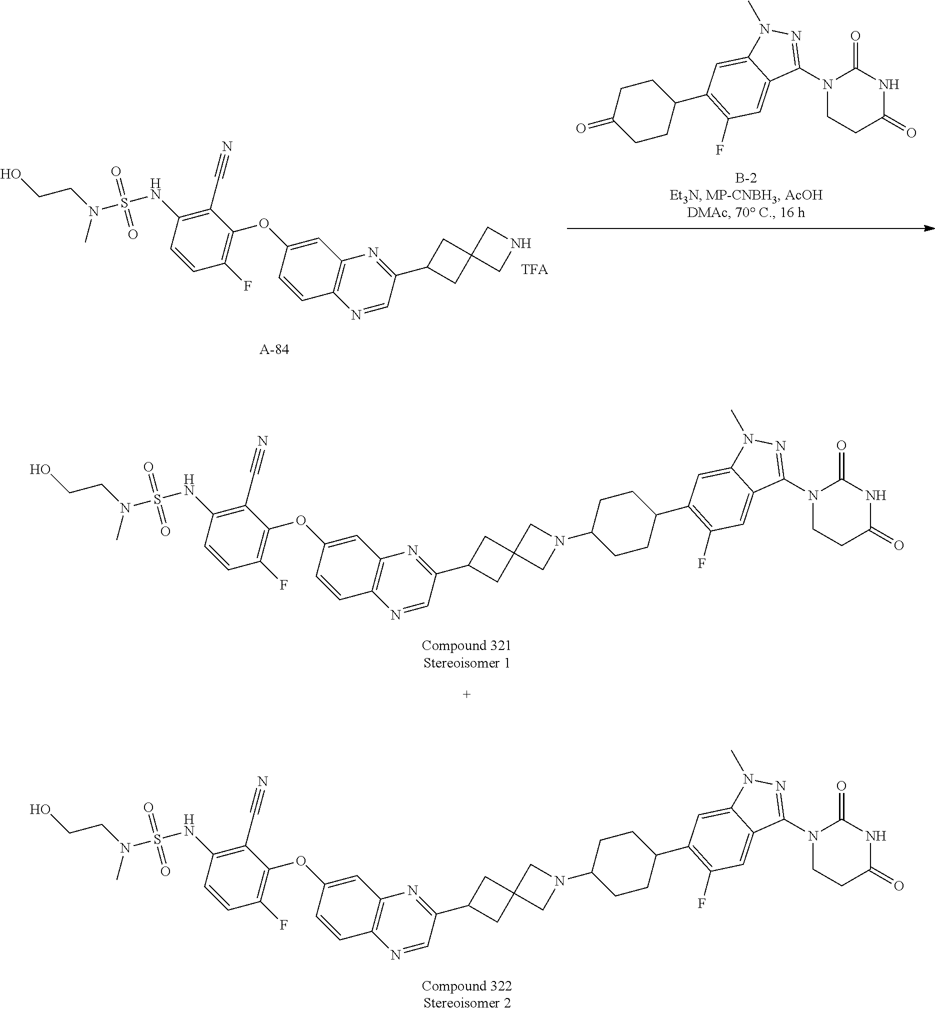

Into a 30 mL glass vial with pressure released cap containing a well-stirred solution of 2-(2-azaspiro[3.3]heptan-6-yl)-7-[2-cyano-6-fluoro-3-[[2-hydroxyethyl(methyl)sulfamoyl]amino]phenoxy]quinoxaline (A-84) (300 mg, 348.94 mol, TFA salt) in DMAc (5 mL) was added triethylamine (1.40 mmol, 194.54 μL) at room temperature and stirred for 30 min. Subsequently, 1-[5-fluoro-1-methyl-6-(4-oxocyclohexyl)indazol-3-yl]hexahydropyrimidine-2,4-dione (B-2) (130.26 mg, 348.94 mol) and AcOH (348.94 mol, 19.95 μL) were added to the reaction mixture and stirred at room temperature for 2 hours. Finally, MP-CNBH₃ (300 mg, 348.94 mol) was added, and the mixture was stirred at 70° C. for 16 hours. After completion of the reaction, the reaction mixture was concentrated under reduced pressure to get a crude residue (Mixture of stereoisomer 1 and stereoisomer 2), which was purified by Perp HPLC [Column: X-Bridge C18 (250 mm*19 mm*5 m), mobile phase A: 10 mM NH₄HCO₃ in milli-Q water; mobile phase B: acetonitrile]. The first eluting fraction at t_{R}=4.65 min by HPLC was lyophilized to afford Compound 321 (assigned as stereoisomer 1).

Peak-1: 7-[2-cyano-6-fluoro-3-[[2-hydroxyethyl(methyl)sulfamoyl]amino]phenoxy]-2-[2-[4-[3-(2,4-dioxohexahydropyrimidin-1-yl)-5-fluoro-1-methyl-indazol-6-yl]cyclohexyl]-2-azaspiro[3.3]heptan-6-yl]quinoxaline (Compound 321, 19 mg, 20.72 mol, 6% yield) as an off-white solid. UPLC-MS (ES+): m/z 855.2 [M+H]⁺. ¹H NMR (400 MHz, DMSO-d₆): δ 10.56 (s, 1H), 9.72 (bs, 1H), 8.77 (s, 1H), 8.13 (d, J=9.20 Hz, 1H), 7.69 (dd, J=2.80, 9.20 Hz, 1H), 7.53 (d, J=6.00 Hz, 1H), 7.48-7.25 (m, 3H), 7.13 (d, J=2.40 Hz, 1H), 4.65-3.98 (m, 7H), 3.92-3.80 (m, 3H), 3.50 (t, J=6.40 Hz, 2H), 3.16-3.00 (m, 3H), 3.90-3.82 (m, 1H), 2.79-2.61 (m, 9H), 2.11-1.93 (m, 4H), 1.67-1.56 (m, 2H), 1.38-1.23 (m, 2H).

Similarly, the second eluted fraction at t_{R}=4.88 min by HPLC was lyophilized to afford Compound 322 (assigned as stereoisomer 2).

Peak-2: 7-[2-cyano-6-fluoro-3-[[2-hydroxyethyl(methyl)sulfamoyl]amino]phenoxy]-2-[2-[4-[3-(2,4-dioxohexahydropyrimidin-1-yl)-5-fluoro-1-methyl-indazol-6-yl]cyclohexyl]-2-azaspiro[3.3]heptan-6-yl]quinoxaline (Compound 322, 20 mg, 22.76 mol, 6% yield) as an off-white solid. UPLC-MS (ES+): m/z 854.9 [M+H]⁺. ¹H NMR (400 MHz, DMSO-d₆): δ 5 10.56 (s, 1H), 9.52 (bs, 1H), 8.77 (s, 1H), 8.12 (d, J=9.20 Hz, 1H), 7.68 (dd, J=2.80, 9.20 Hz, 1H), 7.49-7.25 (m, 4H), 7.15 (s, 1H), 4.75-4.10 (m, 4H), 4.01 (s, 3H), 3.91 (t, J=6.80 Hz, 2H), 3.85-3.80 (m, 1H), 3.50 (t, J=6.40 Hz, 2H), 3.10-2.90 (m, 4H), 2.76 (t, J=6.80 Hz, 2H), 2.70-2.58 (m, 6H), 2.00-1.55 (in, 10H).

COMPOUND TABLE 1

| Structure | Cmpd |
|---|---|
| 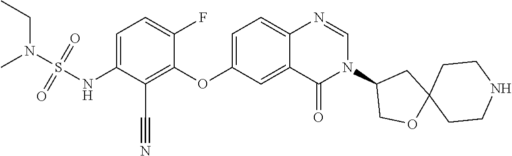 [SMILES: CCN(C)S(=O)(=O)Nc1ccc(F)c(Oc2ccc3ncn([C@@H]4COC5(CCNCC5)C4)c(=O)c3c2)c1C#N] | A-1 |
| 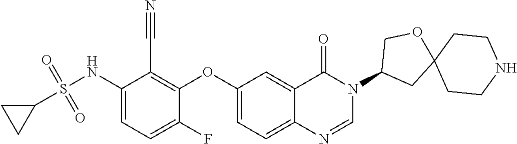 [SMILES: N#Cc1c(NS(=O)(=O)C2CC2)ccc(F)c1Oc1ccc2ncn([C@H]3COC4(CCNCC4)C3)c(=O)c2c1] | A-2 |
| 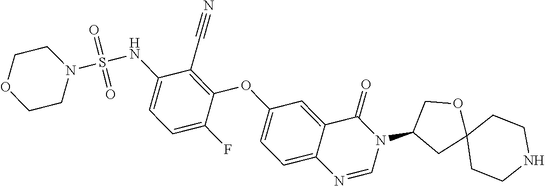 [SMILES: N#Cc1c(NS(=O)(=O)N2CCOCC2)ccc(F)c1Oc1ccc2ncn([C@H]3COC4(CCNCC4)C3)c(=O)c2c1] | A-3 |
| 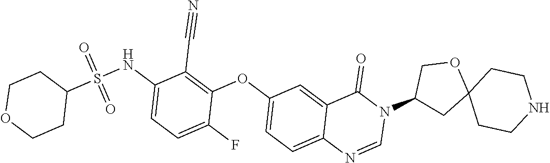 [SMILES: N#Cc1c(NS(=O)(=O)C2CCOCC2)ccc(F)c1Oc1ccc2ncn([C@H]3COC4(CCNCC4)C3)c(=O)c2c1] | A-4 |
| 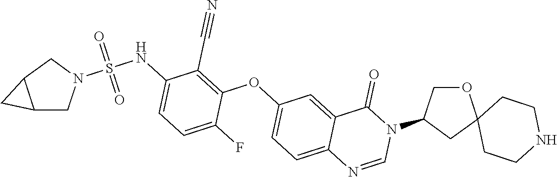 [SMILES: N#Cc1c(NS(=O)(=O)N2CC3CC3C2)ccc(F)c1Oc1ccc2ncn([C@H]3COC4(CCNCC4)C3)c(=O)c2c1] | A-5 |
| 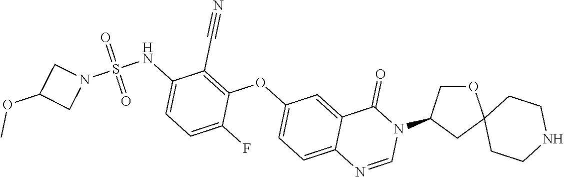 [SMILES: COC1CN(S(=O)(=O)Nc2ccc(F)c(Oc3ccc4ncn([C@H]5COC6(CCNCC6)C5)c(=O)c4c3)c2C#N)C1] | A-6 |
| 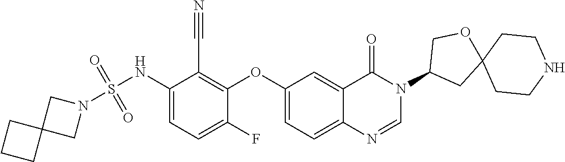 [SMILES: N#Cc1c(NS(=O)(=O)N2CC3(CCC3)C2)ccc(F)c1Oc1ccc2ncn([C@H]3COC4(CCNCC4)C3)c(=O)c2c1] | A-7 |
| 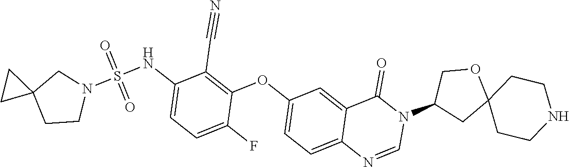 [SMILES: N#Cc1c(NS(=O)(=O)N2CCC3(CC3)C2)ccc(F)c1Oc1ccc2ncn([C@H]3COC4(CCNCC4)C3)c(=O)c2c1] | A-8 |
| 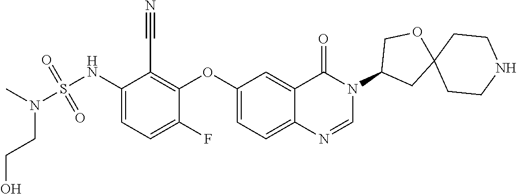 [SMILES: CN(CCO)S(=O)(=O)Nc1ccc(F)c(Oc2ccc3ncn([C@H]4COC5(CCNCC5)C4)c(=O)c3c2)c1C#N] | A-9 |
| 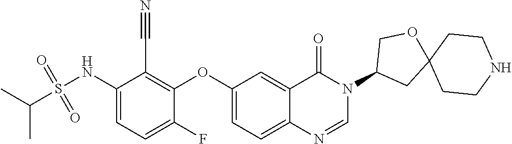 [SMILES: CC(C)S(=O)(=O)Nc1ccc(F)c(Oc2ccc3ncn([C@H]4COC5(CCNCC5)C4)c(=O)c3c2)c1C#N] | A-10 |
| 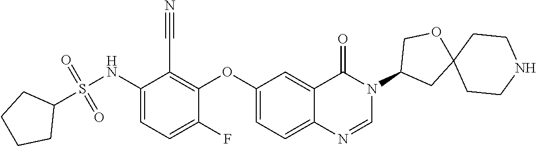 [SMILES: N#Cc1c(NS(=O)(=O)C2CCCC2)ccc(F)c1Oc1ccc2ncn([C@H]3COC4(CCNCC4)C3)c(=O)c2c1] | A-11 |
| 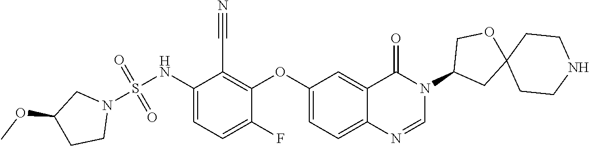 [SMILES: CO[C@@H]1CCN(S(=O)(=O)Nc2ccc(F)c(Oc3ccc4ncn([C@H]5COC6(CCNCC6)C5)c(=O)c4c3)c2C#N)C1] | A-12 |
| 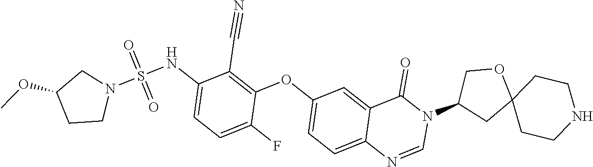 [SMILES: CO[C@H]1CCN(S(=O)(=O)Nc2ccc(F)c(Oc3ccc4ncn([C@H]5COC6(CCNCC6)C5)c(=O)c4c3)c2C#N)C1] | A-13 |
| 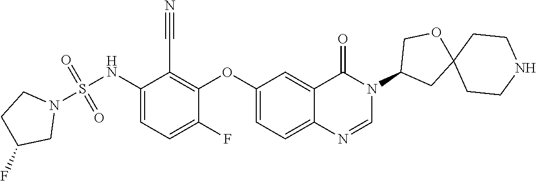 [SMILES: N#Cc1c(NS(=O)(=O)N2CC[C@@H](F)C2)ccc(F)c1Oc1ccc2ncn([C@H]3COC4(CCNCC4)C3)c(=O)c2c1] | A-14 |
| 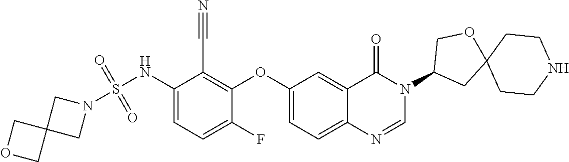 [SMILES: N#Cc1c(NS(=O)(=O)N2CC3(COC3)C2)ccc(F)c1Oc1ccc2ncn([C@H]3COC4(CCNCC4)C3)c(=O)c2c1] | A-15 |
| 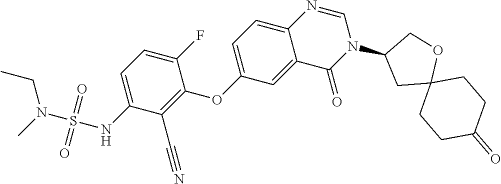 [SMILES: CCN(C)S(=O)(=O)Nc1ccc(F)c(Oc2ccc3ncn([C@H]4COC5(CCC(=O)CC5)C4)c(=O)c3c2)c1C#N] | A-16 |
| 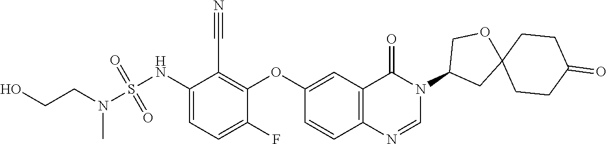 [SMILES: CN(CCO)S(=O)(=O)Nc1ccc(F)c(Oc2ccc3ncn([C@H]4COC5(CCC(=O)CC5)C4)c(=O)c3c2)c1C#N] | A-17 |
| 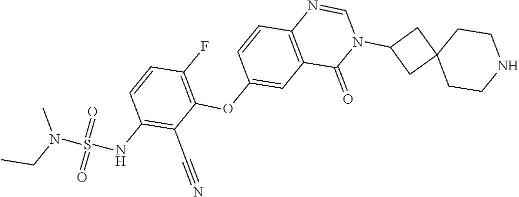 [SMILES: CCN(C)S(=O)(=O)Nc1ccc(F)c(Oc2ccc3ncn(C4CC5(CCNCC5)C4)c(=O)c3c2)c1C#N] | A-18 |
| 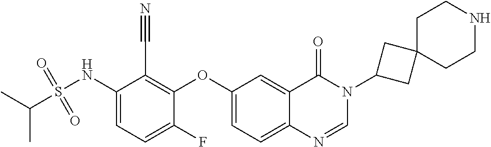 [SMILES: CC(C)S(=O)(=O)Nc1ccc(F)c(Oc2ccc3ncn(C4CC5(CCNCC5)C4)c(=O)c3c2)c1C#N] | A-19 |
| 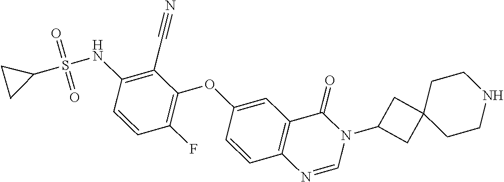 [SMILES: N#Cc1c(NS(=O)(=O)C2CC2)ccc(F)c1Oc1ccc2ncn(C3CC4(CCNCC4)C3)c(=O)c2c1] | A-20 |
| 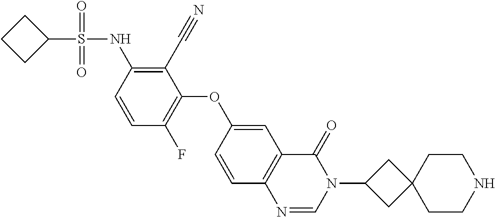 [SMILES: N#Cc1c(NS(=O)(=O)C2CCC2)ccc(F)c1Oc1ccc2ncn(C3CC4(CCNCC4)C3)c(=O)c2c1] | A-21 |
| 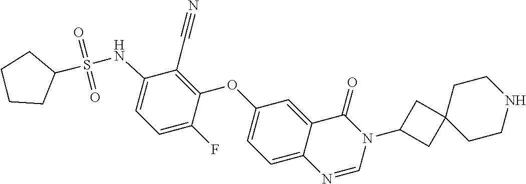 [SMILES: N#Cc1c(NS(=O)(=O)C2CCCC2)ccc(F)c1Oc1ccc2ncn(C3CC4(CCNCC4)C3)c(=O)c2c1] | A-22 |
| 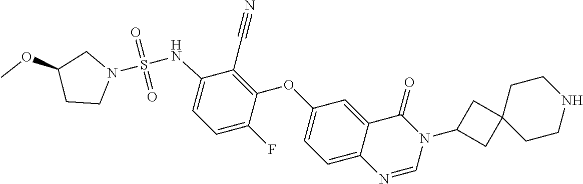 [SMILES: CO[C@@H]1CCN(S(=O)(=O)Nc2ccc(F)c(Oc3ccc4ncn(C5CC6(CCNCC6)C5)c(=O)c4c3)c2C#N)C1] | A-23 |
| 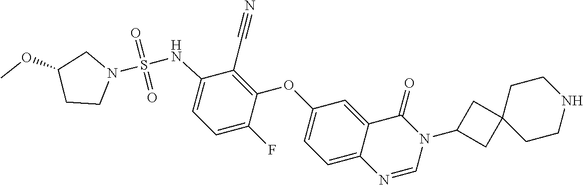 [SMILES: CO[C@H]1CCN(S(=O)(=O)Nc2ccc(F)c(Oc3ccc4ncn(C5CC6(CCNCC6)C5)c(=O)c4c3)c2C#N)C1] | A-24 |
| 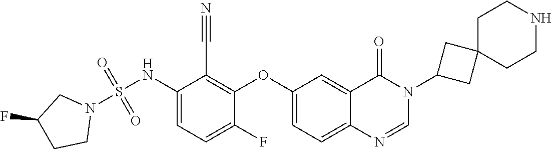 [SMILES: N#Cc1c(NS(=O)(=O)N2CC[C@@H](F)C2)ccc(F)c1Oc1ccc2ncn(C3CC4(CCNCC4)C3)c(=O)c2c1] | A-25 |
| 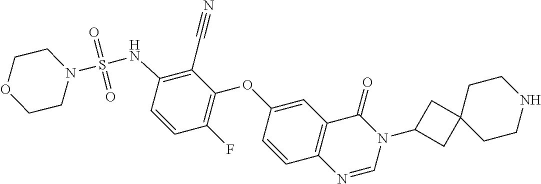 [SMILES: N#Cc1c(NS(=O)(=O)N2CCOCC2)ccc(F)c1Oc1ccc2ncn(C3CC4(CCNCC4)C3)c(=O)c2c1] | A-26 |
| 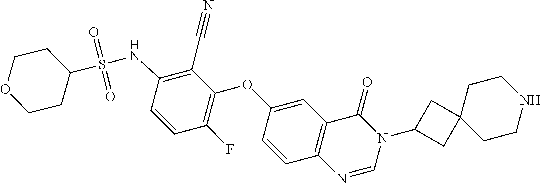 [SMILES: N#Cc1c(NS(=O)(=O)C2CCOCC2)ccc(F)c1Oc1ccc2ncn(C3CC4(CCNCC4)C3)c(=O)c2c1] | A-27 |
| 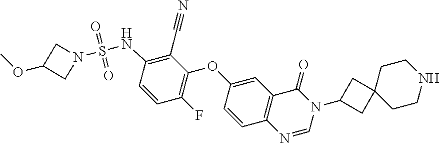 [SMILES: COC1CN(S(=O)(=O)Nc2ccc(F)c(Oc3ccc4ncn(C5CC6(CCNCC6)C5)c(=O)c4c3)c2C#N)C1] | A-28 |
| 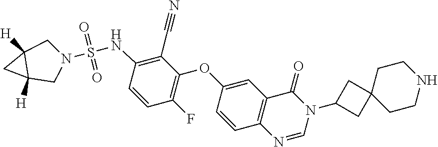 [SMILES: N#Cc1c(NS(=O)(=O)N2C[C@H]3C[C@H]3C2)ccc(F)c1Oc1ccc2ncn(C3CC4(CCNCC4)C3)c(=O)c2c1] | A-29 |
| 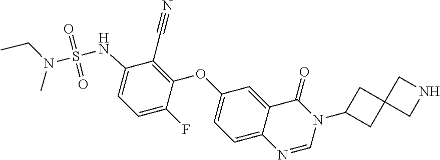 [SMILES: CCN(C)S(=O)(=O)Nc1ccc(F)c(Oc2ccc3ncn(C4CC5(CNC5)C4)c(=O)c3c2)c1C#N] | A-30 |
| 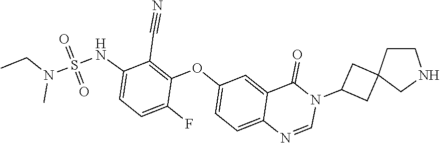 [SMILES: CCN(C)S(=O)(=O)Nc1ccc(F)c(Oc2ccc3ncn(C4CC5(CCNC5)C4)c(=O)c3c2)c1C#N] | A-31 |
| 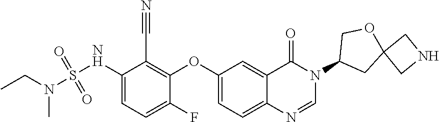 [SMILES: CCN(C)S(=O)(=O)Nc1ccc(F)c(Oc2ccc3ncn([C@H]4COC5(CNC5)C4)c(=O)c3c2)c1C#N] | A-32 |
| 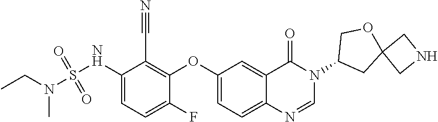 [SMILES: CCN(C)S(=O)(=O)Nc1ccc(F)c(Oc2ccc3ncn([C@@H]4COC5(CNC5)C4)c(=O)c3c2)c1C#N] | A-33 |
| 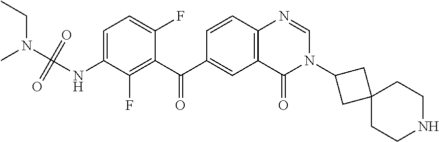 | A-34 |
| 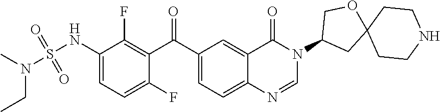 [SMILES: CCN(C)S(=O)(=O)Nc1ccc(F)c(C(=O)c2ccc3ncn([C@H]4COC5(CCNCC5)C4)c(=O)c3c2)c1F] | A-35 |
| 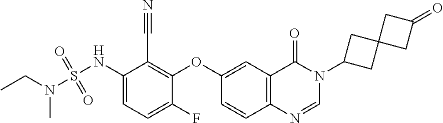 [SMILES: CCN(C)S(=O)(=O)Nc1ccc(F)c(Oc2ccc3ncn(C4CC5(CC(=O)C5)C4)c(=O)c3c2)c1C#N] | A-36 |
| 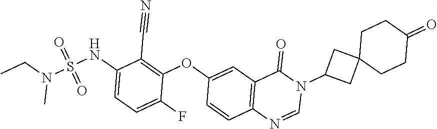 [SMILES: CCN(C)S(=O)(=O)Nc1ccc(F)c(Oc2ccc3ncn(C4CC5(CCC(=O)CC5)C4)c(=O)c3c2)c1C#N] | A-37 |
| 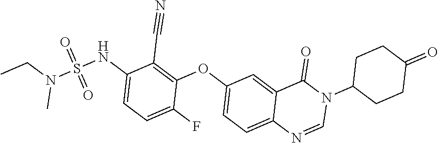 [SMILES: CCN(C)S(=O)(=O)Nc1ccc(F)c(Oc2ccc3ncn(C4CCC(=O)CC4)c(=O)c3c2)c1C#N] | A-38 |
| 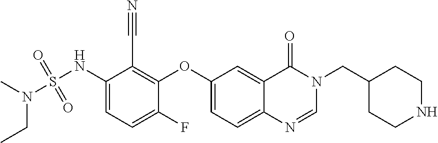 [SMILES: CCN(C)S(=O)(=O)Nc1ccc(F)c(Oc2ccc3ncn(CC4CCNCC4)c(=O)c3c2)c1C#N] | A-39 |
| 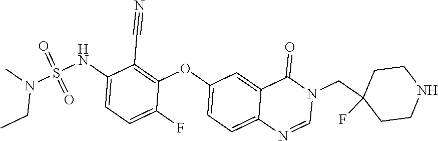 [SMILES: CCN(C)S(=O)(=O)Nc1ccc(F)c(Oc2ccc3ncn(CC4(F)CCNCC4)c(=O)c3c2)c1C#N] | A-40 |
| 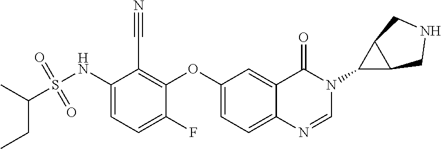 [SMILES: CCC(C)S(=O)(=O)Nc1ccc(F)c(Oc2ccc3ncn([C@H]4[C@@H]5CNC[C@@H]54)c(=O)c3c2)c1C#N] | A-41 |
| 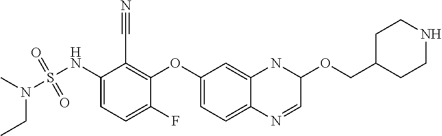 [SMILES: CCN(C)S(=O)(=O)Nc1ccc(F)c(Oc2ccc3c(c2)NC(OCC2CCNCC2)C=N3)c1C#N] | A-42 |
| 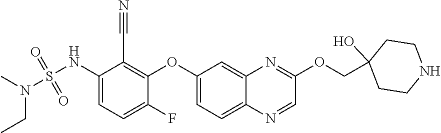 [SMILES: CCN(C)S(=O)(=O)Nc1ccc(F)c(Oc2ccc3ncc(OCC4(O)CCNCC4)nc3c2)c1C#N] | A-43 |
| 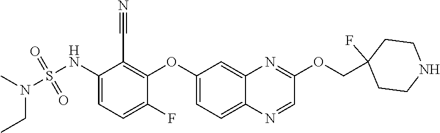 [SMILES: CCN(C)S(=O)(=O)Nc1ccc(F)c(Oc2ccc3ncc(OCC4(F)CCNCC4)nc3c2)c1C#N] | A-44 |
| 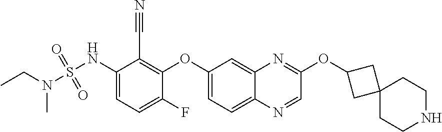 [SMILES: CCN(C)S(=O)(=O)Nc1ccc(F)c(Oc2ccc3ncc(OC4CC5(CCNCC5)C4)nc3c2)c1C#N] | A-45 |
| 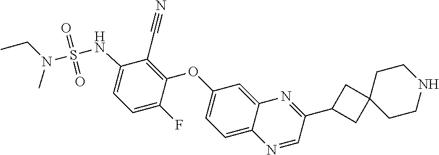 [SMILES: CCN(C)S(=O)(=O)Nc1ccc(F)c(Oc2ccc3ncc(C4CC5(CCNCC5)C4)nc3c2)c1C#N] | A-46 |
| 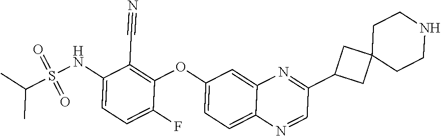 [SMILES: CC(C)S(=O)(=O)Nc1ccc(F)c(Oc2ccc3ncc(C4CC5(CCNCC5)C4)nc3c2)c1C#N] | A-47 |
| 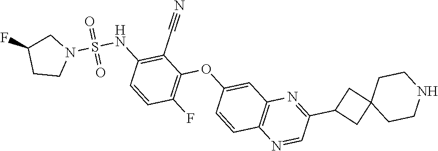 [SMILES: N#Cc1c(NS(=O)(=O)N2CC[C@@H](F)C2)ccc(F)c1Oc1ccc2ncc(C3CC4(CCNCC4)C3)nc2c1] | A-48 |
| 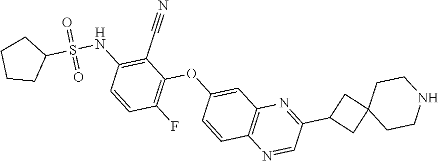 [SMILES: N#Cc1c(NS(=O)(=O)C2CCCC2)ccc(F)c1Oc1ccc2ncc(C3CC4(CCNCC4)C3)nc2c1] | A-49 |
| 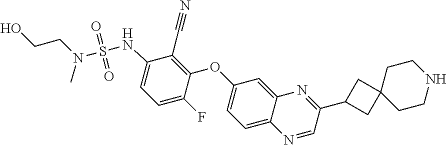 [SMILES: CN(CCO)S(=O)(=O)Nc1ccc(F)c(Oc2ccc3ncc(C4CC5(CCNCC5)C4)nc3c2)c1C#N] | A-50 |
| 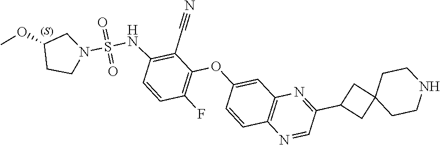 | A-51 |
| 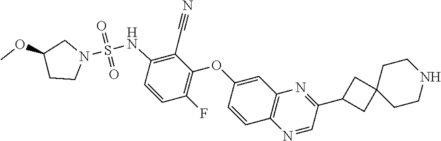 [SMILES: CO[C@@H]1CCN(S(=O)(=O)Nc2ccc(F)c(Oc3ccc4ncc(C5CC6(CCNCC6)C5)nc4c3)c2C#N)C1] | A-52 |
| 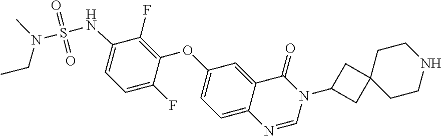 [SMILES: CCN(C)S(=O)(=O)Nc1ccc(F)c(Oc2ccc3ncn(C4CC5(CCNCC5)C4)c(=O)c3c2)c1F] | A-53 |
| 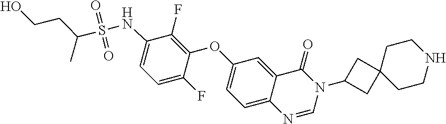 [SMILES: CC(CCO)S(=O)(=O)Nc1ccc(F)c(Oc2ccc3ncn(C4CC5(CCNCC5)C4)c(=O)c3c2)c1F] | A-54 |
| 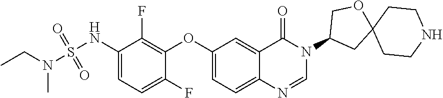 [SMILES: CCN(C)S(=O)(=O)Nc1ccc(F)c(Oc2ccc3ncn([C@H]4COC5(CCNCC5)C4)c(=O)c3c2)c1F] | A-55 |
| 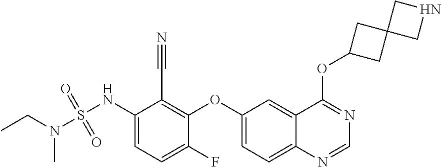 [SMILES: CCN(C)S(=O)(=O)Nc1ccc(F)c(Oc2ccc3ncnc(OC4CC5(CNC5)C4)c3c2)c1C#N] | A-56 |
| 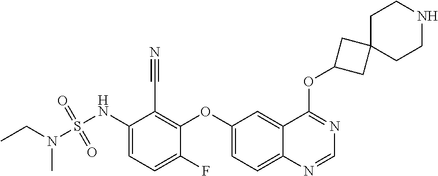 [SMILES: CCN(C)S(=O)(=O)Nc1ccc(F)c(Oc2ccc3ncnc(OC4CC5(CCNCC5)C4)c3c2)c1C#N] | A-57 |
| 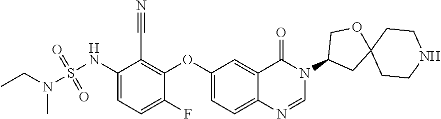 [SMILES: CCN(C)S(=O)(=O)Nc1ccc(F)c(Oc2ccc3ncn([C@H]4COC5(CCNCC5)C4)c(=O)c3c2)c1C#N] | A-58 |
| 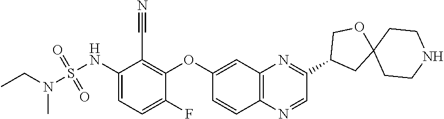 [SMILES: CCN(C)S(=O)(=O)Nc1ccc(F)c(Oc2ccc3ncc([C@@H]4COC5(CCNCC5)C4)nc3c2)c1C#N] | A-59 |
| 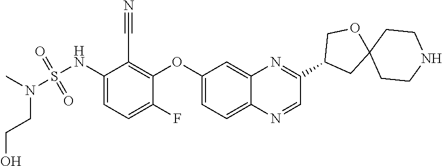 [SMILES: CN(CCO)S(=O)(=O)Nc1ccc(F)c(Oc2ccc3ncc([C@@H]4COC5(CCNCC5)C4)nc3c2)c1C#N] | A-60 |
| 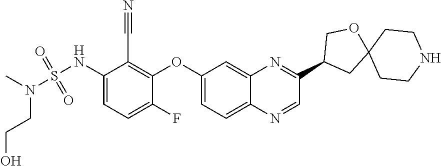 [SMILES: CN(CCO)S(=O)(=O)Nc1ccc(F)c(Oc2ccc3ncc([C@H]4COC5(CCNCC5)C4)nc3c2)c1C#N] | A-61 |
| 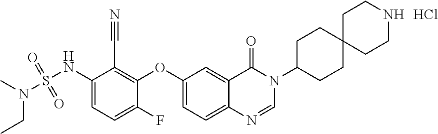 [SMILES: CCN(C)S(=O)(=O)Nc1ccc(F)c(Oc2ccc3ncn(C4CCC5(CCNCC5)CC4)c(=O)c3c2)c1C#N ; Cl] | A-62 |
| 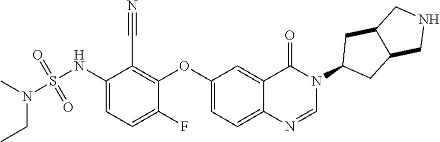 [SMILES: CCN(C)S(=O)(=O)Nc1ccc(F)c(Oc2ccc3ncn([C@H]4C[C@H]5CNC[C@H]5C4)c(=O)c3c2)c1C#N] | A-63 |
| 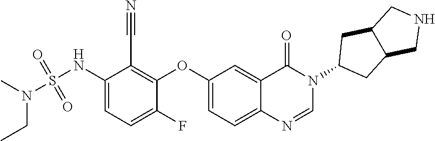 [SMILES: CCN(C)S(=O)(=O)Nc1ccc(F)c(Oc2ccc3ncn([C@@H]4C[C@H]5CNC[C@H]5C4)c(=O)c3c2)c1C#N] | A-64 |
| 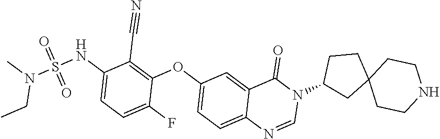 [SMILES: CCN(C)S(=O)(=O)Nc1ccc(F)c(Oc2ccc3ncn([C@@H]4CCC5(CCNCC5)C4)c(=O)c3c2)c1C#N] | A-65 |
| 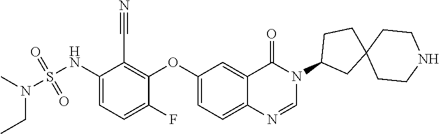 [SMILES: CCN(C)S(=O)(=O)Nc1ccc(F)c(Oc2ccc3ncn([C@H]4CCC5(CCNCC5)C4)c(=O)c3c2)c1C#N] | A-66 |
| 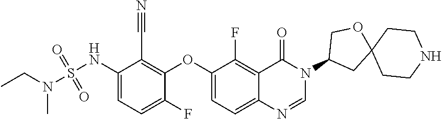 [SMILES: CCN(C)S(=O)(=O)Nc1ccc(F)c(Oc2ccc3ncn([C@H]4COC5(CCNCC5)C4)c(=O)c3c2F)c1C#N] | A-67 |
| 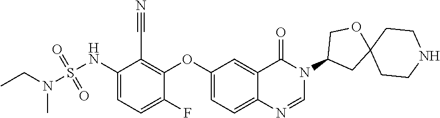 [SMILES: CCN(C)S(=O)(=O)Nc1ccc(F)c(Oc2ccc3ncn([C@H]4COC5(CCNCC5)C4)c(=O)c3c2)c1C#N] | A-68 |
| 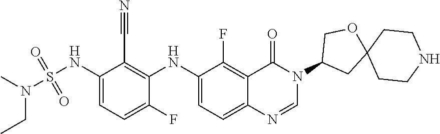 [SMILES: CCN(C)S(=O)(=O)Nc1ccc(F)c(Nc2ccc3ncn([C@H]4COC5(CCNCC5)C4)c(=O)c3c2F)c1C#N] | A-69 |
| 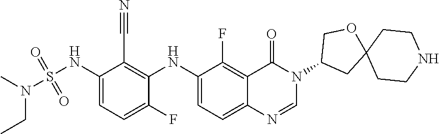 [SMILES: CCN(C)S(=O)(=O)Nc1ccc(F)c(Nc2ccc3ncn([C@@H]4COC5(CCNCC5)C4)c(=O)c3c2F)c1C#N] | A-70 |
| 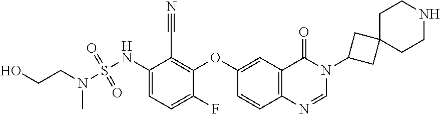 [SMILES: CN(CCO)S(=O)(=O)Nc1ccc(F)c(Oc2ccc3ncn(C4CC5(CCNCC5)C4)c(=O)c3c2)c1C#N] | A-71 |
| 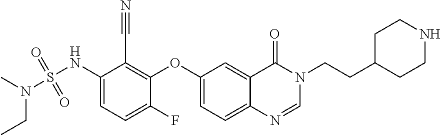 [SMILES: CCN(C)S(=O)(=O)Nc1ccc(F)c(Oc2ccc3ncn(CCC4CCNCC4)c(=O)c3c2)c1C#N] | A-72 |
| 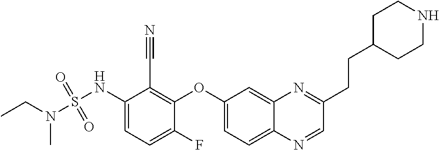 [SMILES: CCN(C)S(=O)(=O)Nc1ccc(F)c(Oc2ccc3ncc(CCC4CCNCC4)nc3c2)c1C#N] | A-73 |
| 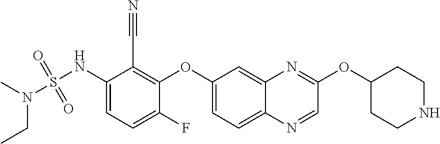 [SMILES: CCN(C)S(=O)(=O)Nc1ccc(F)c(Oc2ccc3ncc(OC4CCNCC4)nc3c2)c1C#N] | A-74 |
| 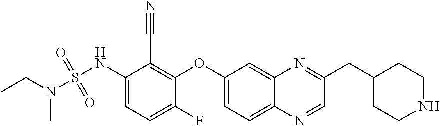 [SMILES: CCN(C)S(=O)(=O)Nc1ccc(F)c(Oc2ccc3ncc(CC4CCNCC4)nc3c2)c1C#N] | A-75 |
| 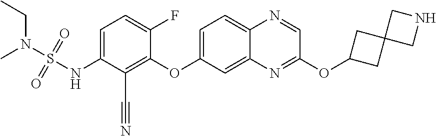 [SMILES: CCN(C)S(=O)(=O)Nc1ccc(F)c(Oc2ccc3ncc(OC4CC5(CNC5)C4)nc3c2)c1C#N] | A-76 |
| 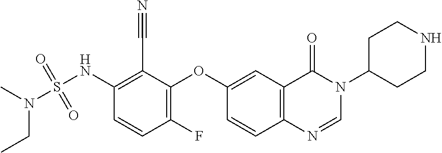 [SMILES: CCN(C)S(=O)(=O)Nc1ccc(F)c(Oc2ccc3ncn(C4CCNCC4)c(=O)c3c2)c1C#N] | A-77 |
| 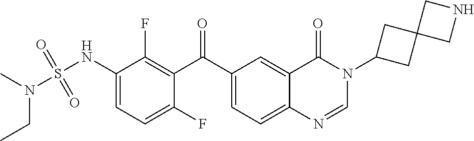 [SMILES: CCN(C)S(=O)(=O)Nc1ccc(F)c(C(=O)c2ccc3ncn(C4CC5(CNC5)C4)c(=O)c3c2)c1F] | A-78 |
| 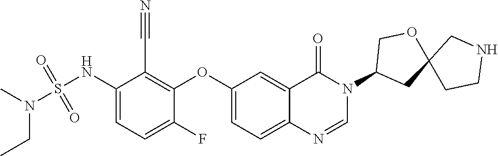 [SMILES: CCN(C)S(=O)(=O)Nc1ccc(F)c(Oc2ccc3ncn([C@H]4CO[C@]5(CCNC5)C4)c(=O)c3c2)c1C#N] | A-79 |
| 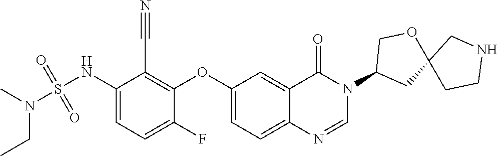 [SMILES: CCN(C)S(=O)(=O)Nc1ccc(F)c(Oc2ccc3ncn([C@H]4CO[C@@]5(CCNC5)C4)c(=O)c3c2)c1C#N] | A-80 |
| 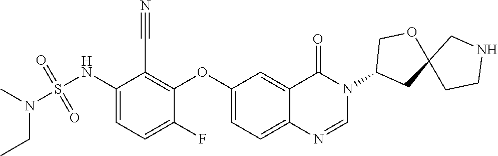 [SMILES: CCN(C)S(=O)(=O)Nc1ccc(F)c(Oc2ccc3ncn([C@@H]4CO[C@]5(CCNC5)C4)c(=O)c3c2)c1C#N] | A-81 |
| 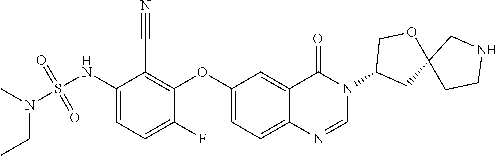 [SMILES: CCN(C)S(=O)(=O)Nc1ccc(F)c(Oc2ccc3ncn([C@@H]4CO[C@@]5(CCNC5)C4)c(=O)c3c2)c1C#N] | A-82 |
| 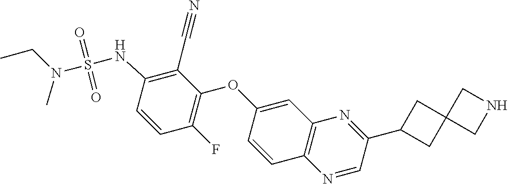 [SMILES: CCN(C)S(=O)(=O)Nc1ccc(F)c(Oc2ccc3ncc(C4CC5(CNC5)C4)nc3c2)c1C#N] | A-83 |
| 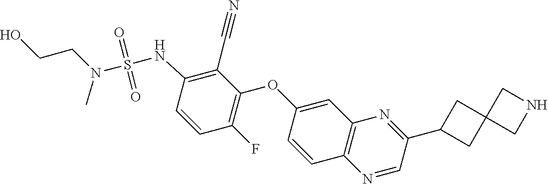 [SMILES: CN(CCO)S(=O)(=O)Nc1ccc(F)c(Oc2ccc3ncc(C4CC5(CNC5)C4)nc3c2)c1C#N] | A-84 |
| 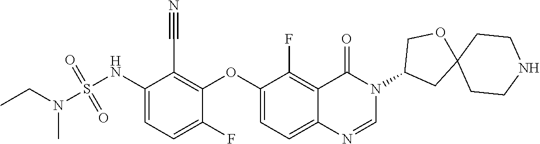 [SMILES: CCN(C)S(=O)(=O)Nc1ccc(F)c(Oc2ccc3ncn([C@@H]4COC5(CCNCC5)C4)c(=O)c3c2F)c1C#N] | A-85 |
| 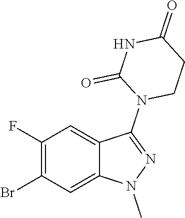 [SMILES: Cn1nc(N2CCC(=O)NC2=O)c2cc(F)c(Br)cc21] | B-1 |
| 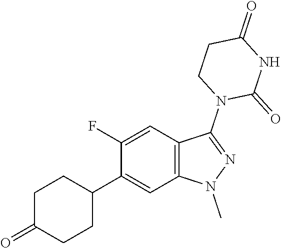 [SMILES: Cn1nc(N2CCC(=O)NC2=O)c2cc(F)c(C3CCC(=O)CC3)cc21] | B-2 |
| 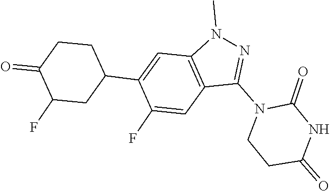 [SMILES: Cn1nc(N2CCC(=O)NC2=O)c2cc(F)c(C3CCC(=O)C(F)C3)cc21] | B-3 |
| 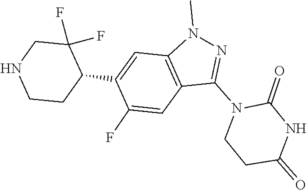 [SMILES: Cn1nc(N2CCC(=O)NC2=O)c2cc(F)c([C@@H]3CCNCC3(F)F)cc21] | B-4 |
| 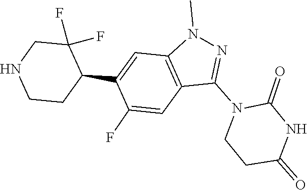 [SMILES: Cn1nc(N2CCC(=O)NC2=O)c2cc(F)c([C@H]3CCNCC3(F)F)cc21] | B-5 |
| 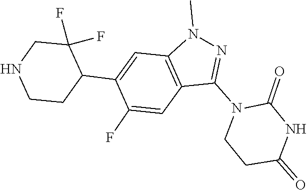 [SMILES: Cn1nc(N2CCC(=O)NC2=O)c2cc(F)c(C3CCNCC3(F)F)cc21] | B-6 |
| 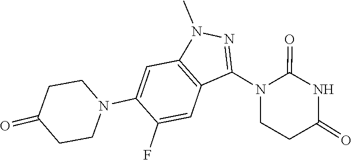 [SMILES: Cn1nc(N2CCC(=O)NC2=O)c2cc(F)c(N3CCC(=O)CC3)cc21] | B-7 |
| 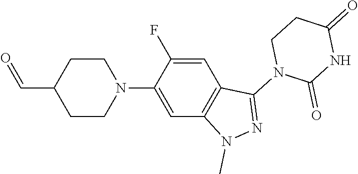 [SMILES: Cn1nc(N2CCC(=O)NC2=O)c2cc(F)c(N3CCC(C=O)CC3)cc21] | B-8 |
| 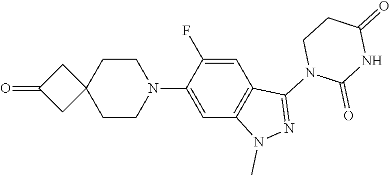 [SMILES: Cn1nc(N2CCC(=O)NC2=O)c2cc(F)c(N3CCC4(CC3)CC(=O)C4)cc21] | B-9 |
| 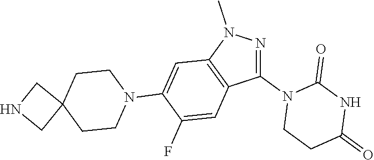 [SMILES: Cn1nc(N2CCC(=O)NC2=O)c2cc(F)c(N3CCC4(CC3)CNC4)cc21] | B-10 |
| 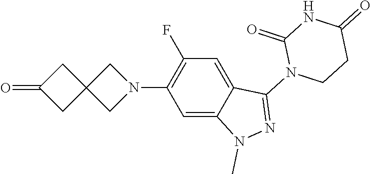 [SMILES: Cn1nc(N2CCC(=O)NC2=O)c2cc(F)c(N3CC4(CC(=O)C4)C3)cc21] | B-11 |
| 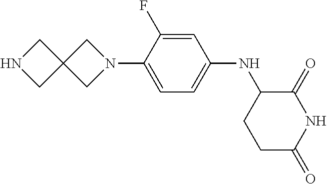 [SMILES: O=C1CCC(Nc2ccc(N3CC4(CNC4)C3)c(F)c2)C(=O)N1] | B-12 |
| 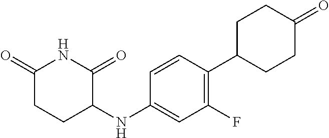 [SMILES: O=C1CCC(c2ccc(NC3CCC(=O)NC3=O)cc2F)CC1] | B-13 |
| 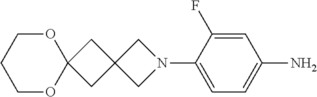 [SMILES: Nc1ccc(N2CC3(C2)CC2(C3)OCCCO2)c(F)c1] | B-14 |
| 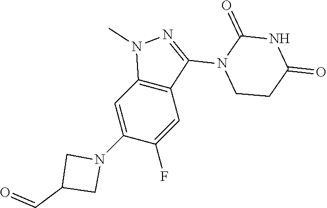 [SMILES: Cn1nc(N2CCC(=O)NC2=O)c2cc(F)c(N3CC(C=O)C3)cc21] | B-15 |
| 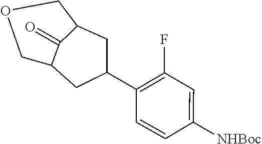 [SMILES: CC(C)(C)OC(=O)Nc1ccc(C2CC3COCC(C2)C3=O)c(F)c1] | B-17 |
| 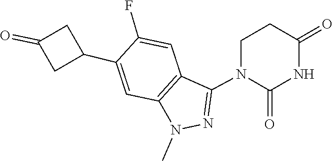 [SMILES: Cn1nc(N2CCC(=O)NC2=O)c2cc(F)c(C3CC(=O)C3)cc21] | B-18 |
| 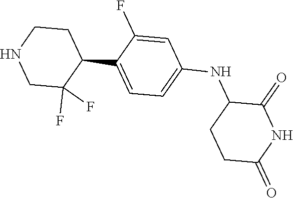 [SMILES: O=C1CCC(Nc2ccc([C@@H]3CCNCC3(F)F)c(F)c2)C(=O)N1] | B-19 |
| 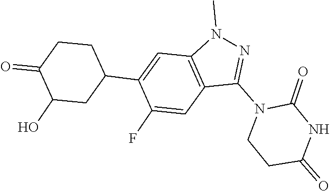 [SMILES: Cn1nc(N2CCC(=O)NC2=O)c2cc(F)c(C3CCC(=O)C(O)C3)cc21] | B-20 |
| 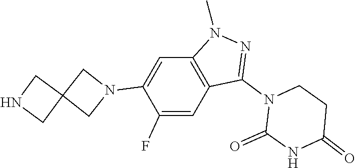 [SMILES: Cn1nc(N2CCC(=O)NC2=O)c2cc(F)c(N3CC4(CNC4)C3)cc21] | B-21 |
| 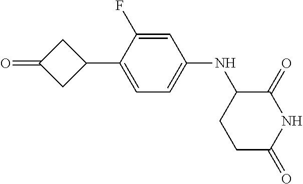 [SMILES: O=C1CC(c2ccc(NC3CCC(=O)NC3=O)cc2F)C1] | B-22 |
| 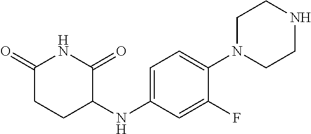 [SMILES: O=C1CCC(Nc2ccc(N3CCNCC3)c(F)c2)C(=O)N1] | B-23 |
| 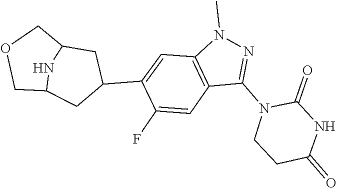 [SMILES: Cn1nc(N2CCC(=O)NC2=O)c2cc(F)c(C3CC4COCC(C3)N4)cc21] | B-24 |
| 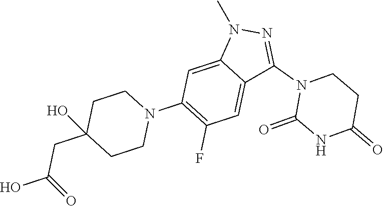 [SMILES: Cn1nc(N2CCC(=O)NC2=O)c2cc(F)c(N3CCC(O)(CC(=O)O)CC3)cc21] | B-25 |
| 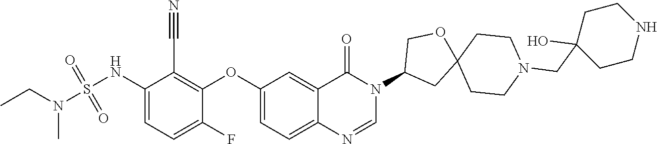 [SMILES: CCN(C)S(=O)(=O)Nc1ccc(F)c(Oc2ccc3ncn([C@H]4COC5(CCN(CC6(O)CCNCC6)CC5)C4)c(=O)c3c2)c1C#N] | C-1 |
| 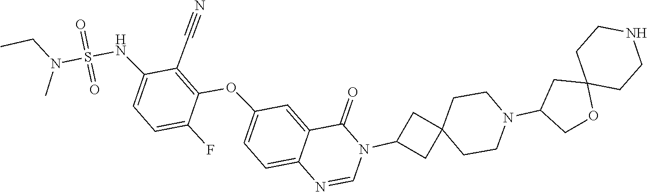 [SMILES: CCN(C)S(=O)(=O)Nc1ccc(F)c(Oc2ccc3ncn(C4CC5(CCN(C6COC7(CCNCC7)C6)CC5)C4)c(=O)c3c2)c1C#N] | C-2 |
| 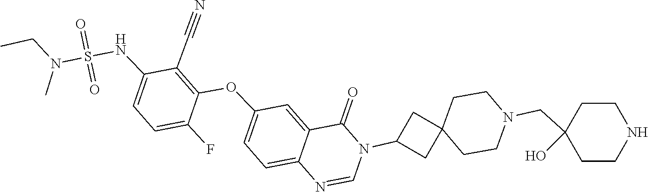 [SMILES: CCN(C)S(=O)(=O)Nc1ccc(F)c(Oc2ccc3ncn(C4CC5(CCN(CC6(O)CCNCC6)CC5)C4)c(=O)c3c2)c1C#N] | C-3 |
| 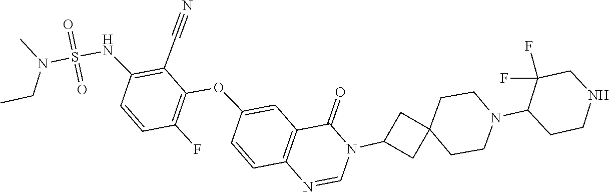 [SMILES: CCN(C)S(=O)(=O)Nc1ccc(F)c(Oc2ccc3ncn(C4CC5(CCN(C6CCNCC6(F)F)CC5)C4)c(=O)c3c2)c1C#N] | C-4 |
| 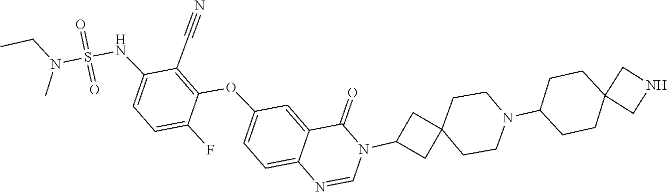 [SMILES: CCN(C)S(=O)(=O)Nc1ccc(F)c(Oc2ccc3ncn(C4CC5(CCN(C6CCC7(CC6)CNC7)CC5)C4)c(=O)c3c2)c1C#N] | C-5 |
| 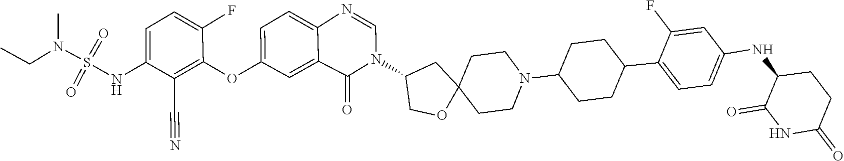 [SMILES: CCN(C)S(=O)(=O)Nc1ccc(F)c(Oc2ccc3ncn([C@H]4COC5(CCN(C6CCC(c7ccc(N[C@H]8CCC(=O)NC8=O)cc7F)CC6)CC5)C4)c(=O)c3c2)c1C#N] Compound 3 Stereoisomer 1 | Compound 3 |
| 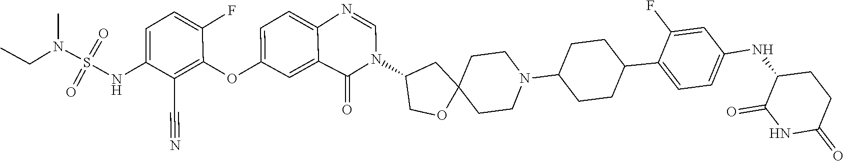 [SMILES: CCN(C)S(=O)(=O)Nc1ccc(F)c(Oc2ccc3ncn([C@H]4COC5(CCN(C6CCC(c7ccc(N[C@@H]8CCC(=O)NC8=O)cc7F)CC6)CC5)C4)c(=O)c3c2)c1C#N] Compound 4 Stereoisomer 2 | Compound 4 |
| 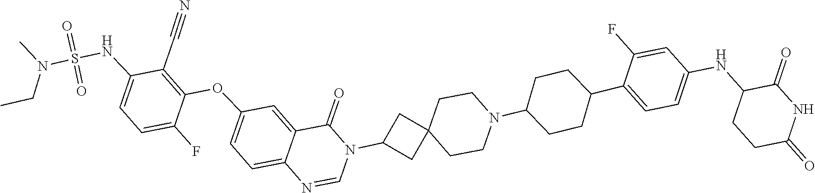 [SMILES: CCN(C)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1C#N] Compound 6 Stereoisomer 1 | Compound 6 |
| 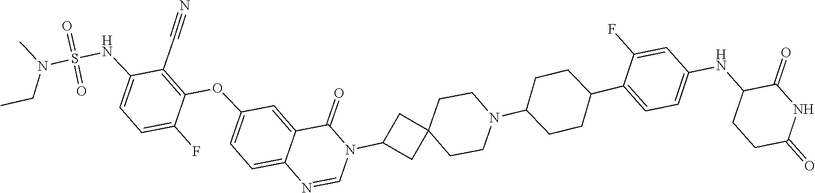 [SMILES: CCN(C)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1C#N] Compound 7 Stereoisomer 2 | Compound 7 |
| 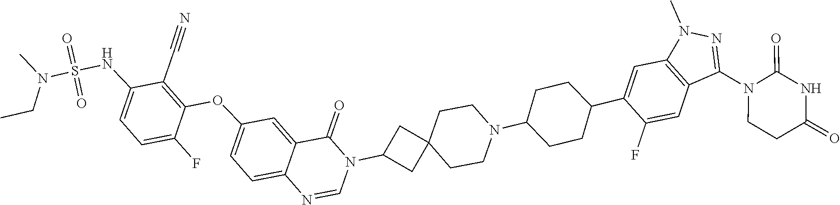 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6)CC5)C4)c(=O)c3c2)c1C#N] Compound 8 Stereoisomer 1 | Compound 8 |
| 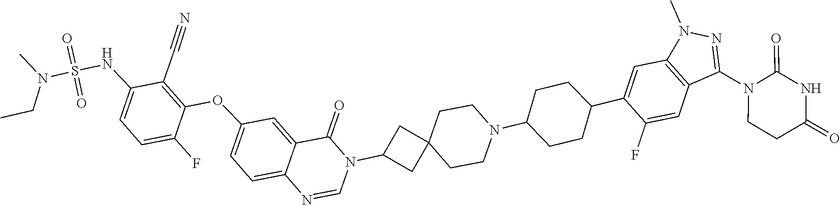 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6)CC5)C4)c(=O)c3c2)c1C#N] Compound 9 Stereoisomer 2 | Compound 9 |
| 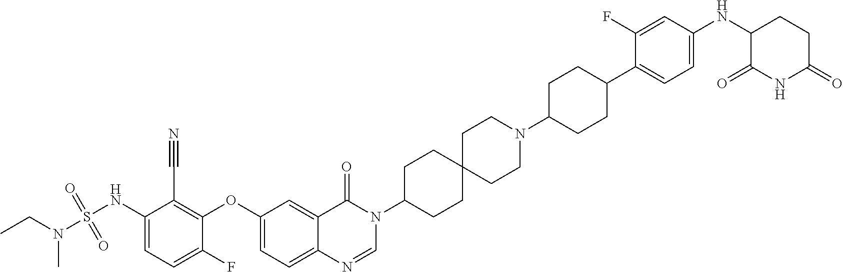 [SMILES: CCN(C)S(=O)(=O)Nc1ccc(F)c(Oc2ccc3ncn(C4CCC5(CC4)CCN(C4CCC(c6ccc(NC7CCC(=O)NC7=O)cc6F)CC4)CC5)c(=O)c3c2)c1C#N] Compound 10 Stereoisomer 1 | Compound 10 |
| 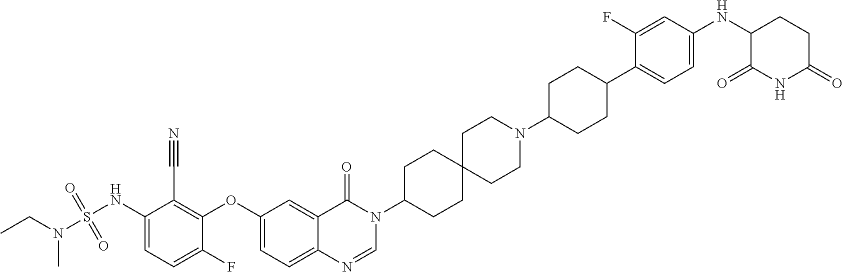 [SMILES: CCN(C)S(=O)(=O)Nc1ccc(F)c(Oc2ccc3ncn(C4CCC5(CC4)CCN(C4CCC(c6ccc(NC7CCC(=O)NC7=O)cc6F)CC4)CC5)c(=O)c3c2)c1C#N] Compound 11 Stereoisomer 2 | Compound 11 |
| 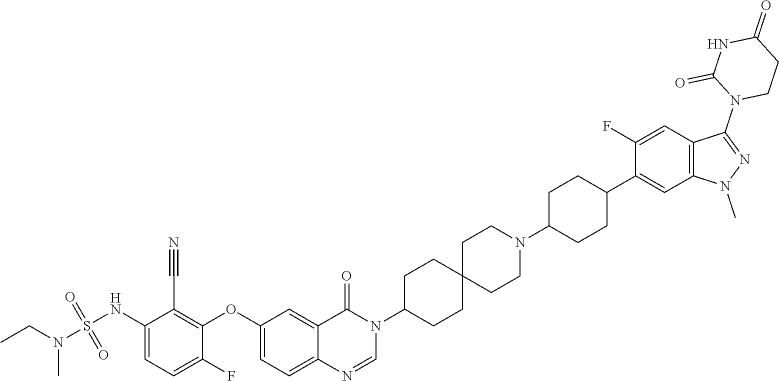 [SMILES: CCN(C)S(=O)(=O)Nc1ccc(F)c(Oc2ccc3ncn(C4CCC5(CC4)CCN(C4CCC(c6cc7c(cc6F)c(N6CCC(=O)NC6=O)nn7C)CC4)CC5)c(=O)c3c2)c1C#N] Compound 12 Stereoisomer 1 | Compound 12 |
| 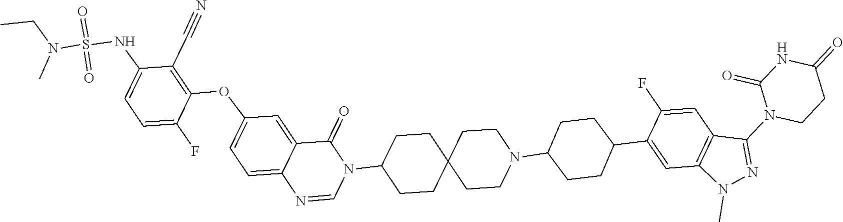 [SMILES: CCN(C)S(=O)(=O)Nc1ccc(F)c(Oc2ccc3ncn(C4CCC5(CC4)CCN(C4CCC(c6cc7c(cc6F)c(N6CCC(=O)NC6=O)nn7C)CC4)CC5)c(=O)c3c2)c1C#N] Compound 13 Stereoisomer 2 | Compound 13 |
| 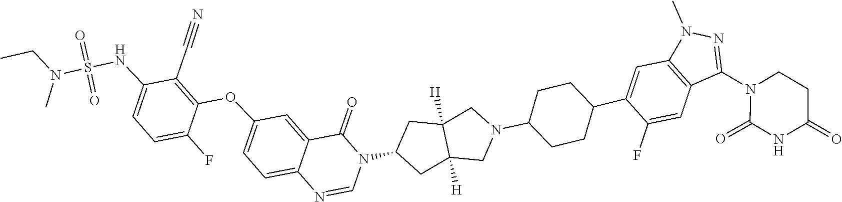 [SMILES: CCN(C)S(=O)(=O)Nc1ccc(F)c(Oc2ccc3ncn([C@H]4C[C@@H]5CN(C6CCC(c7cc8c(cc7F)c(N7CCC(=O)NC7=O)nn8C)CC6)C[C@@H]5C4)c(=O)c3c2)c1C#N] Compound 14 Stereoisomer 1 | Compound 14 |
| 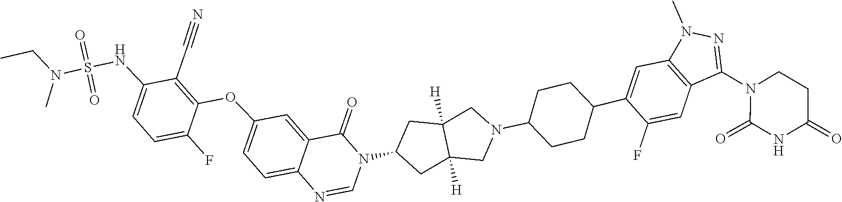 [SMILES: CCN(C)S(=O)(=O)Nc1ccc(F)c(Oc2ccc3ncn([C@H]4C[C@@H]5CN(C6CCC(c7cc8c(cc7F)c(N7CCC(=O)NC7=O)nn8C)CC6)C[C@@H]5C4)c(=O)c3c2)c1C#N] Compound 15 Stereoisomer 2 | Compound 15 |
| 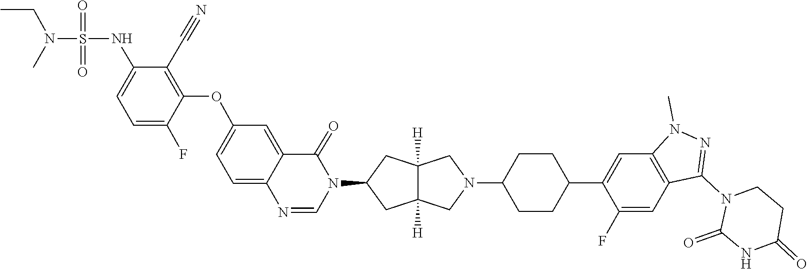 [SMILES: CCN(C)S(=O)(=O)Nc1ccc(F)c(Oc2ccc3ncn([C@@H]4C[C@@H]5CN(C6CCC(c7cc8c(cc7F)c(N7CCC(=O)NC7=O)nn8C)CC6)C[C@@H]5C4)c(=O)c3c2)c1C#N] Compound 16 Stereoisomer 1 | Compound 16 |
| 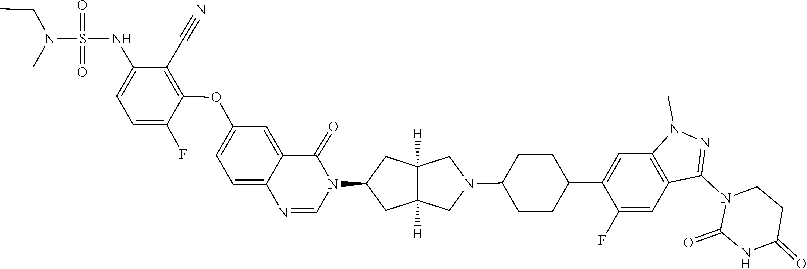 [SMILES: CCN(C)S(=O)(=O)Nc1ccc(F)c(Oc2ccc3ncn([C@@H]4C[C@@H]5CN(C6CCC(c7cc8c(cc7F)c(N7CCC(=O)NC7=O)nn8C)CC6)C[C@@H]5C4)c(=O)c3c2)c1C#N] Compound 17 Stereoisomer 2 | Compound 17 |
| 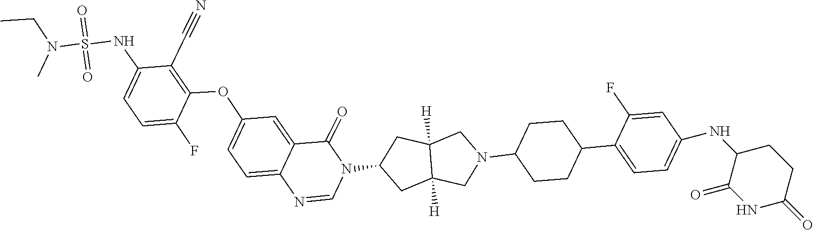 [SMILES: CCN(C)S(=O)(=O)Nc1ccc(F)c(Oc2ccc3ncn([C@H]4C[C@@H]5CN(C6CCC(c7ccc(NC8CCC(=O)NC8=O)cc7F)CC6)C[C@@H]5C4)c(=O)c3c2)c1C#N] Compound 18 Stereoisomer 1 | Compound 18 |
| 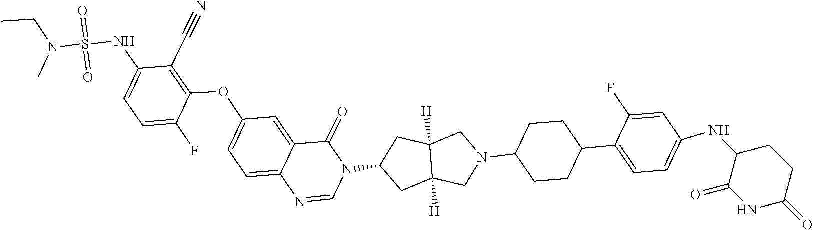 [SMILES: CCN(C)S(=O)(=O)Nc1ccc(F)c(Oc2ccc3ncn([C@H]4C[C@@H]5CN(C6CCC(c7ccc(NC8CCC(=O)NC8=O)cc7F)CC6)C[C@@H]5C4)c(=O)c3c2)c1C#N] Compound 19 Stereoisomer 2 | Compound 19 |
| 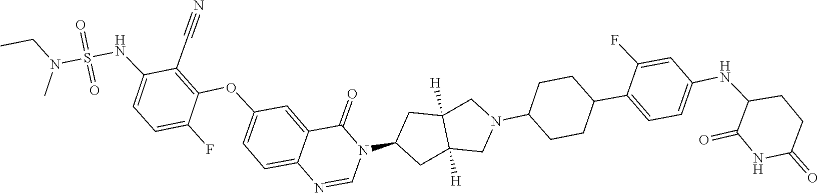 [SMILES: CCN(C)S(=O)(=O)Nc1ccc(F)c(Oc2ccc3ncn([C@@H]4C[C@@H]5CN(C6CCC(c7ccc(NC8CCC(=O)NC8=O)cc7F)CC6)C[C@@H]5C4)c(=O)c3c2)c1C#N] Compound 20 Stereoisomer 1 | Compound 20 |
| 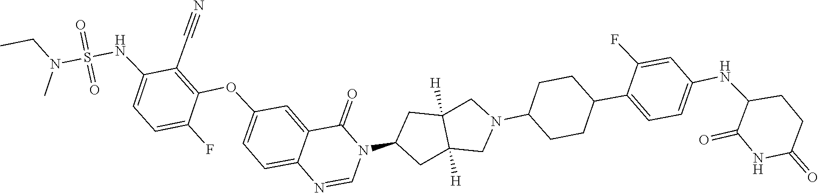 [SMILES: CCN(C)S(=O)(=O)Nc1ccc(F)c(Oc2ccc3ncn([C@@H]4C[C@@H]5CN(C6CCC(c7ccc(NC8CCC(=O)NC8=O)cc7F)CC6)C[C@@H]5C4)c(=O)c3c2)c1C#N] Compound 21 Stereoisomer 2 | Compound 21 |
| 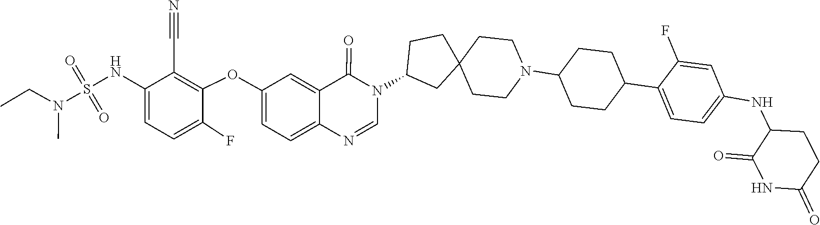 [SMILES: CCN(C)S(=O)(=O)Nc1ccc(F)c(Oc2ccc3ncn([C@@H]4CCC5(CCN(C6CCC(c7ccc(NC8CCC(=O)NC8=O)cc7F)CC6)CC5)C4)c(=O)c3c2)c1C#N] Compound 22 Stereoisomer 1 | Compound 22 |
| 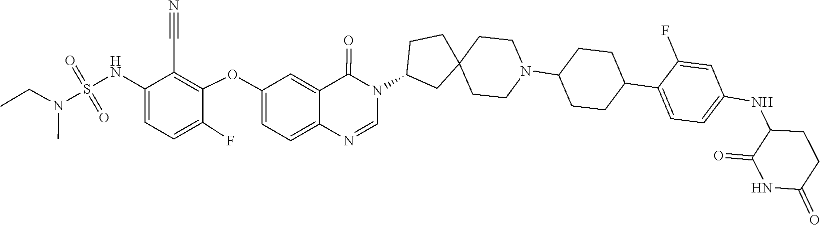 [SMILES: CCN(C)S(=O)(=O)Nc1ccc(F)c(Oc2ccc3ncn([C@@H]4CCC5(CCN(C6CCC(c7ccc(NC8CCC(=O)NC8=O)cc7F)CC6)CC5)C4)c(=O)c3c2)c1C#N] Compound 23 Stereoisomer 2 | Compound 23 |
| 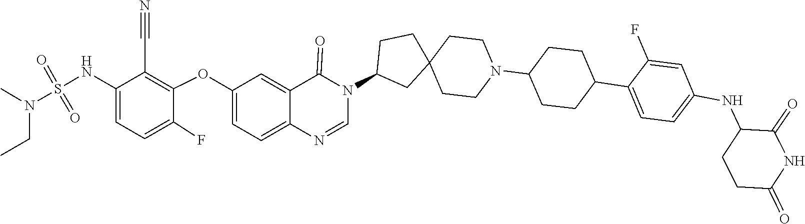 [SMILES: CCN(C)S(=O)(=O)Nc1ccc(F)c(Oc2ccc3ncn([C@H]4CCC5(CCN(C6CCC(c7ccc(NC8CCC(=O)NC8=O)cc7F)CC6)CC5)C4)c(=O)c3c2)c1C#N] Compound 24 Stereoisomer 1 | Compound 24 |
| 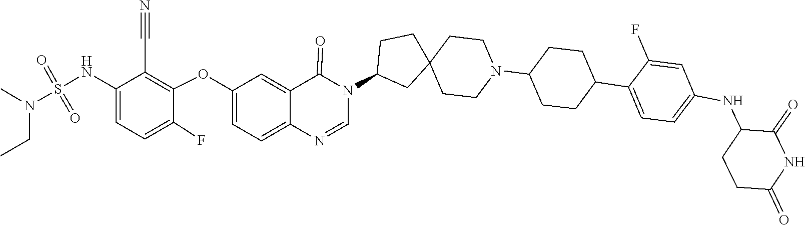 [SMILES: CCN(C)S(=O)(=O)Nc1ccc(F)c(Oc2ccc3ncn([C@H]4CCC5(CCN(C6CCC(c7ccc(NC8CCC(=O)NC8=O)cc7F)CC6)CC5)C4)c(=O)c3c2)c1C#N] Compound 25 Stereoisomer 2 | Compound 25 |
| 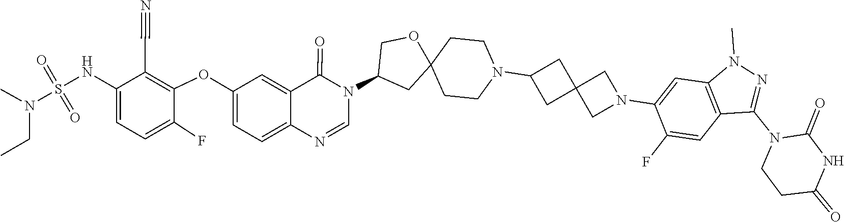 [SMILES: CCN(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N] Compound 26 | Compound 26 |
| 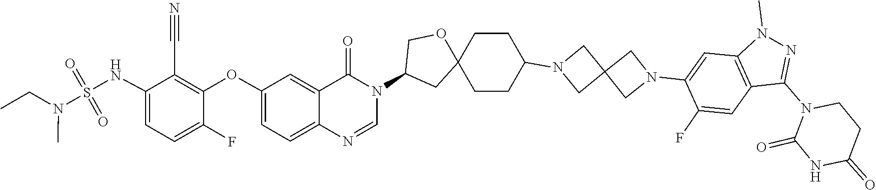 [SMILES: CCN(C)S(=O)(=O)Nc1ccc(F)c(Oc2ccc3ncn([C@H]4COC5(CCC(N6CC7(CN(c8cc9c(cc8F)c(N8CCC(=O)NC8=O)nn9C)C7)C6)CC5)C4)c(=O)c3c2)c1C#N] Compound 27 | Compound 27 |
| 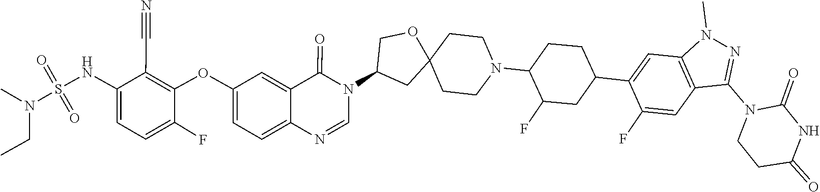 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6F)CC5)C4)c(=O)c3c2)c1C#N] Compound 28 Stereoisomer 1 | Compound 28 |
| 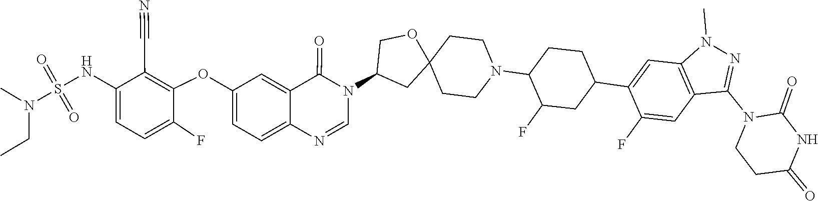 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6F)CC5)C4)c(=O)c3c2)c1C#N] Compound 29 Stereoisomer 2 | Compound 29 |
| 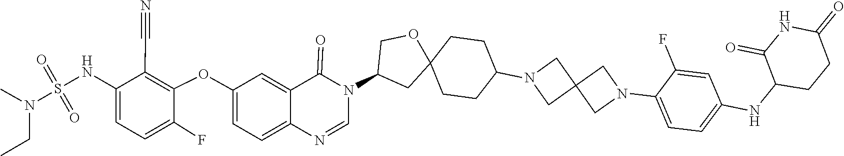 [SMILES: CCN(C)S(=O)(=O)Nc1ccc(F)c(Oc2ccc3ncn([C@H]4COC5(CCC(N6CC7(CN(c8ccc(NC9CCC(=O)NC9=O)cc8F)C7)C6)CC5)C4)c(=O)c3c2)c1C#N] Compound 30 | Compound 30 |
| 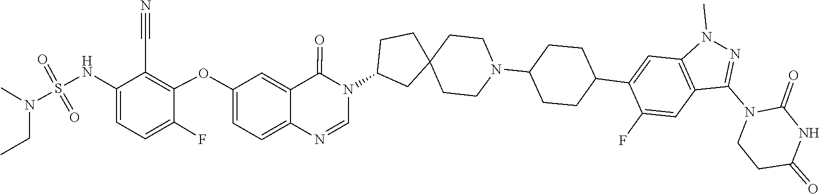 [SMILES: CCN(C)S(=O)(=O)Nc1ccc(F)c(Oc2ccc3ncn([C@@H]4CCC5(CCN(C6CCC(c7cc8c(cc7F)c(N7CCC(=O)NC7=O)nn8C)CC6)CC5)C4)c(=O)c3c2)c1C#N] Compound 31 Stereoisomer 1 | Compound 31 |
| 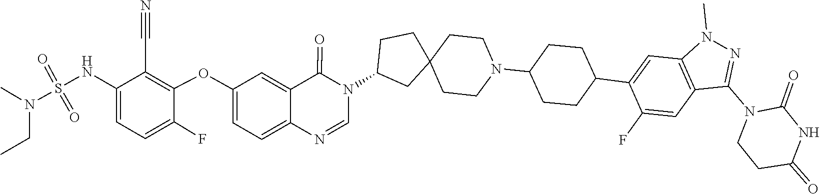 [SMILES: CCN(C)S(=O)(=O)Nc1ccc(F)c(Oc2ccc3ncn([C@@H]4CCC5(CCN(C6CCC(c7cc8c(cc7F)c(N7CCC(=O)NC7=O)nn8C)CC6)CC5)C4)c(=O)c3c2)c1C#N] Compound 32 Stereoisomer 2 | Compound 32 |
| 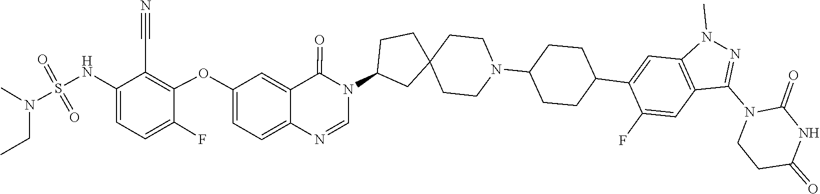 [SMILES: CCN(C)S(=O)(=O)Nc1ccc(F)c(Oc2ccc3ncn([C@H]4CCC5(CCN(C6CCC(c7cc8c(cc7F)c(N7CCC(=O)NC7=O)nn8C)CC6)CC5)C4)c(=O)c3c2)c1C#N] Compound 33 Stereoisomer 1 | Compound 33 |
| 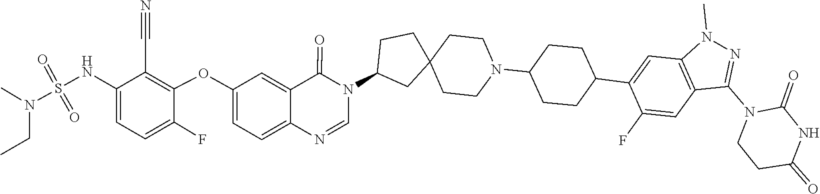 [SMILES: CCN(C)S(=O)(=O)Nc1ccc(F)c(Oc2ccc3ncn([C@H]4CCC5(CCN(C6CCC(c7cc8c(cc7F)c(N7CCC(=O)NC7=O)nn8C)CC6)CC5)C4)c(=O)c3c2)c1C#N] Compound 34 Stereoisomer 2 | Compound 34 |
| 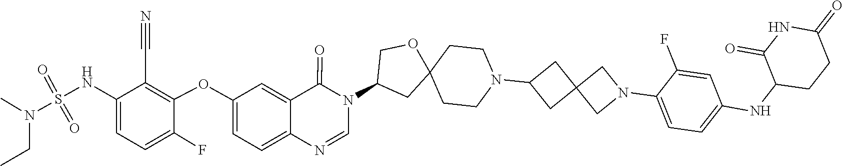 [SMILES: CCN(C)S(=O)(=O)Nc1ccc(F)c(Oc2ccc3ncn([C@H]4COC5(CCN(C6CC7(C6)CN(c6ccc(NC8CCC(=O)NC8=O)cc6F)C7)CC5)C4)c(=O)c3c2)c1C#N] Compound 35 | Compound 35 |
| 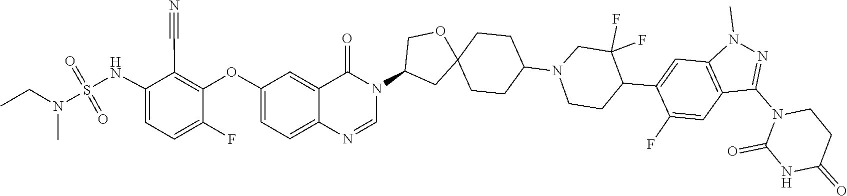 [SMILES: CCN(C)S(=O)(=O)Nc1ccc(F)c(Oc2ccc3ncn([C@H]4COC5(CCC(N6CCC(c7cc8c(cc7F)c(N7CCC(=O)NC7=O)nn8C)C(F)(F)C6)CC5)C4)c(=O)c3c2)c1C#N] Compound 36 Stereoisomer 1 | Compound 36 |
| 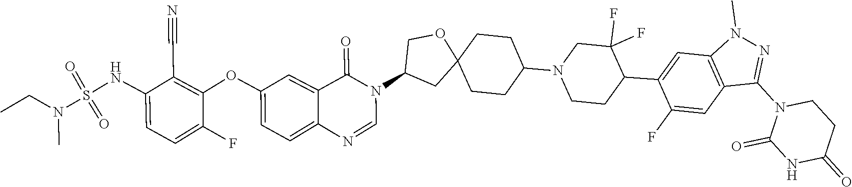 [SMILES: CCN(C)S(=O)(=O)Nc1ccc(F)c(Oc2ccc3ncn([C@H]4COC5(CCC(N6CCC(c7cc8c(cc7F)c(N7CCC(=O)NC7=O)nn8C)C(F)(F)C6)CC5)C4)c(=O)c3c2)c1C#N] Compound 37 Stereoisomer 2 | Compound 37 |
| 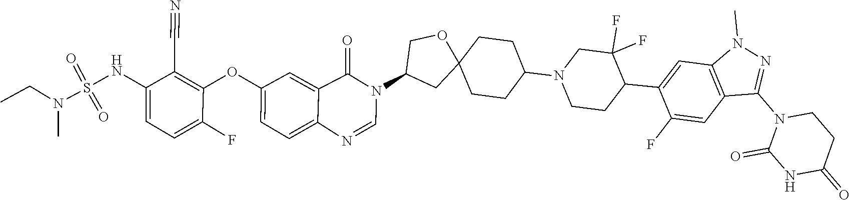 [SMILES: CCN(C)S(=O)(=O)Nc1ccc(F)c(Oc2ccc3ncn([C@H]4COC5(CCC(N6CCC(c7cc8c(cc7F)c(N7CCC(=O)NC7=O)nn8C)C(F)(F)C6)CC5)C4)c(=O)c3c2)c1C#N] Compound 38 Stereoisomer 3 | Compound 38 |
| 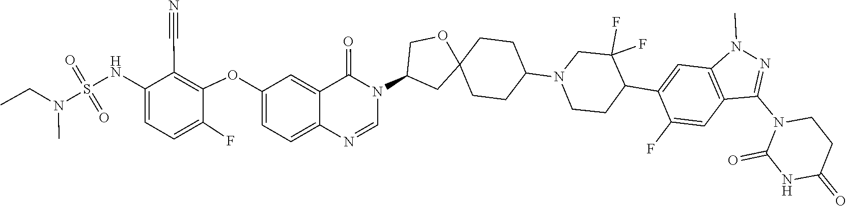 [SMILES: CCN(C)S(=O)(=O)Nc1ccc(F)c(Oc2ccc3ncn([C@H]4COC5(CCC(N6CCC(c7cc8c(cc7F)c(N7CCC(=O)NC7=O)nn8C)C(F)(F)C6)CC5)C4)c(=O)c3c2)c1C#N] Compound 39 Stereoisomer 4 | Compound 39 |
| 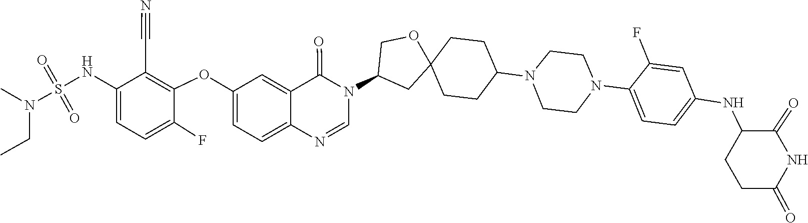 [SMILES: CCN(C)S(=O)(=O)Nc1ccc(F)c(Oc2ccc3ncn([C@H]4COC5(CCC(N6CCN(c7ccc(NC8CCC(=O)NC8=O)cc7F)CC6)CC5)C4)c(=O)c3c2)c1C#N] Compound 40 | Compound 40 |
| 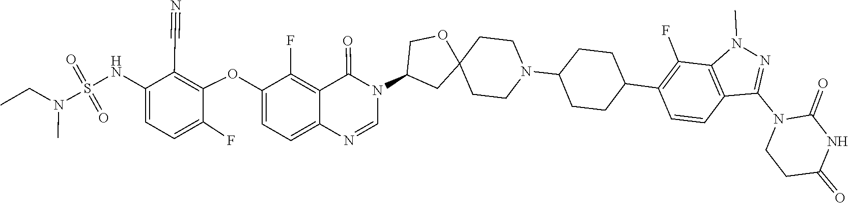 [SMILES: CCN(C)S(=O)(=O)Nc1ccc(F)c(Oc2ccc3ncn([C@H]4COC5(CCN(C6CCC(c7ccc8c(N9CCC(=O)NC9=O)nn(C)c8c7F)CC6)CC5)C4)c(=O)c3c2F)c1C#N] Compound 41 Stereoisomer 1 | Compound 41 |
| 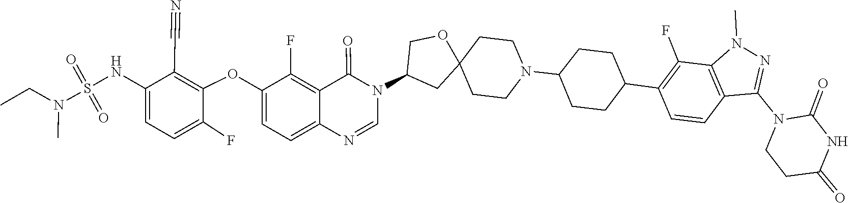 [SMILES: CCN(C)S(=O)(=O)Nc1ccc(F)c(Oc2ccc3ncn([C@H]4COC5(CCN(C6CCC(c7ccc8c(N9CCC(=O)NC9=O)nn(C)c8c7F)CC6)CC5)C4)c(=O)c3c2F)c1C#N] Compound 42 Stereoisomer 2 | Compound 42 |
| 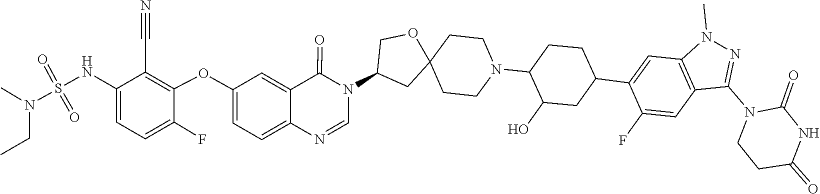 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6O)CC5)C4)c(=O)c3c2)c1C#N] Compound 43 Stereoisomer 1 | Compound 43 |
| 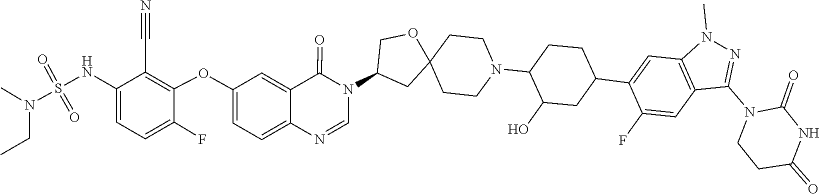 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6O)CC5)C4)c(=O)c3c2)c1C#N] Compound 44 Stereoisomer 2 | Compound 44 |
| 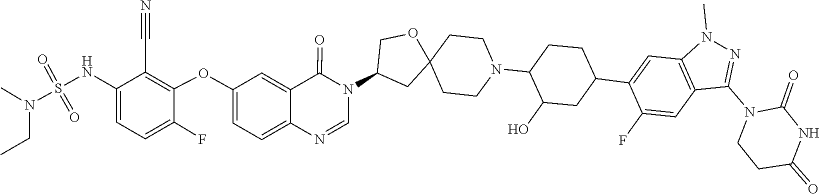 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6O)CC5)C4)c(=O)c3c2)c1C#N] Compound 45 Stereoisomer 3 | Compound 45 |
| 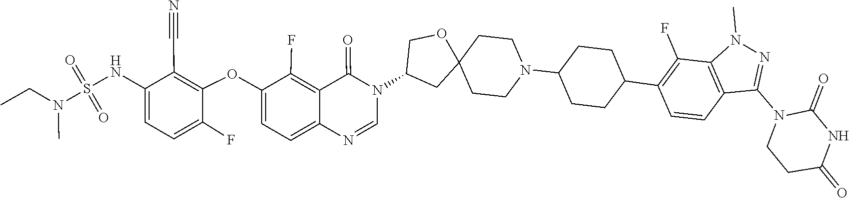 [SMILES: CCN(C)S(=O)(=O)Nc1ccc(F)c(Oc2ccc3ncn([C@@H]4COC5(CCN(C6CCC(c7ccc8c(N9CCC(=O)NC9=O)nn(C)c8c7F)CC6)CC5)C4)c(=O)c3c2F)c1C#N] Compound 46 Stereoisomer 1 | Compound 46 |
| 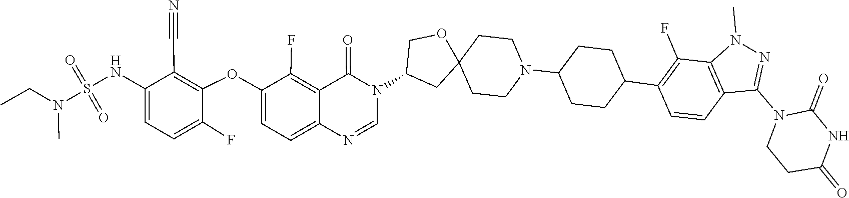 [SMILES: CCN(C)S(=O)(=O)Nc1ccc(F)c(Oc2ccc3ncn([C@@H]4COC5(CCN(C6CCC(c7ccc8c(N9CCC(=O)NC9=O)nn(C)c8c7F)CC6)CC5)C4)c(=O)c3c2F)c1C#N] Compound 47 Stereoisomer 2 | Compound 47 |
| 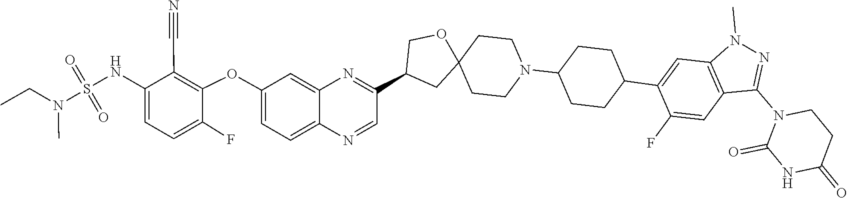 [SMILES: CCN(C)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6)CC5)C4)nc3c2)c1C#N] Compound 48 Stereoisomer 1 | Compound 48 |
| 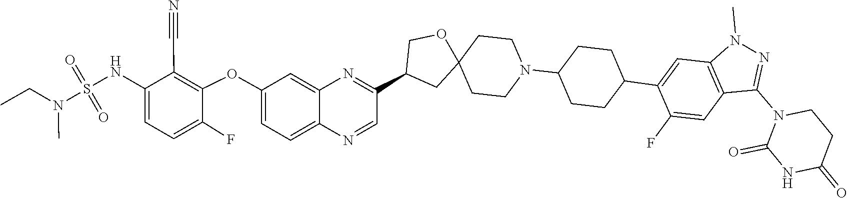 [SMILES: CCN(C)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6)CC5)C4)nc3c2)c1C#N] Compound 49 Stereoisomer 2 | Compound 49 |
| 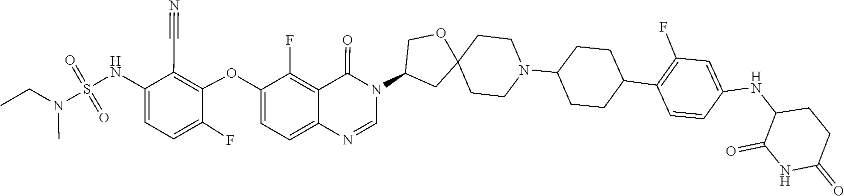 [SMILES: CCN(C)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2F)c1C#N] Compound 50 Stereoisomer 1 | Compound 50 |
| 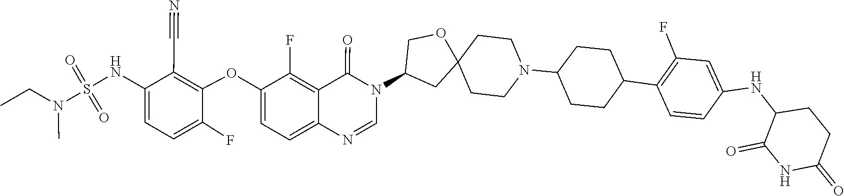 [SMILES: CCN(C)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2F)c1C#N] Compound 51 Stereoisomer 2 | Compound 51 |
| 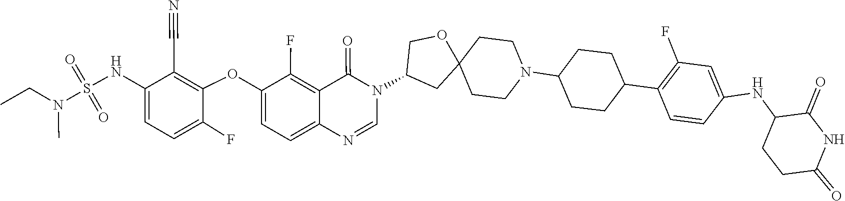 [SMILES: CCN(C)S(=O)(=O)Nc1ccc(F)c(Oc2ccc3ncn([C@@H]4COC5(CCN(C6CCC(c7ccc(NC8CCC(=O)NC8=O)cc7F)CC6)CC5)C4)c(=O)c3c2F)c1C#N] Compound 52 Stereoisomer 1 | Compound 52 |
| 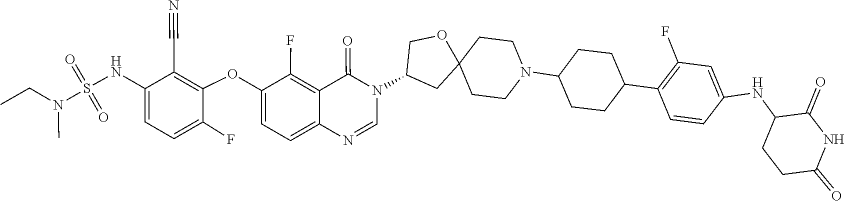 [SMILES: CCN(C)S(=O)(=O)Nc1ccc(F)c(Oc2ccc3ncn([C@@H]4COC5(CCN(C6CCC(c7ccc(NC8CCC(=O)NC8=O)cc7F)CC6)CC5)C4)c(=O)c3c2F)c1C#N] Compound 53 Stereoisomer 2 | Compound 53 |
| 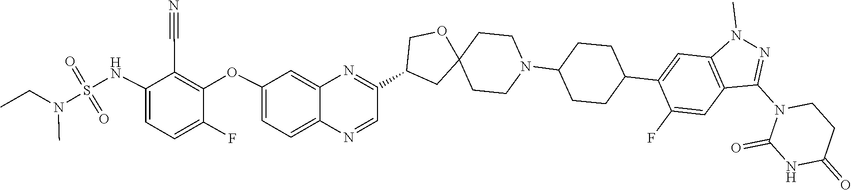 [SMILES: CCN(C)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6)CC5)C4)nc3c2)c1C#N] Compound 54 Stereoisomer 1 | Compound 54 |
| 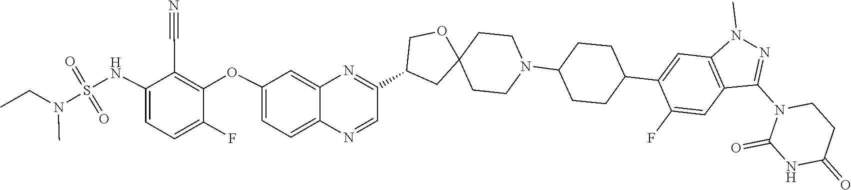 [SMILES: CCN(C)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6)CC5)C4)nc3c2)c1C#N] Compound 55 Stereoisomer 2 | Compound 55 |
| 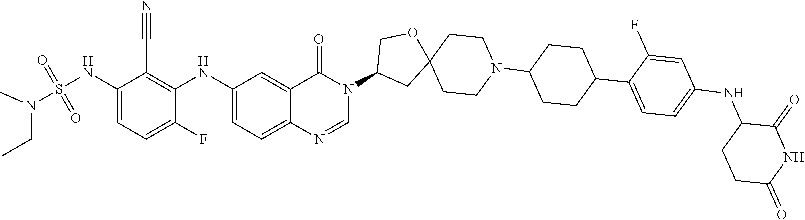 [SMILES: CCN(C)S(=O)(=O)Nc1ccc(F)c(Nc2ccc3ncn([C@H]4COC5(CCN(C6CCC(c7ccc(NC8CCC(=O)NC8=O)cc7F)CC6)CC5)C4)c(=O)c3c2)c1C#N] Compound 56 Stereoisomer 1 | Compound 56 |
| 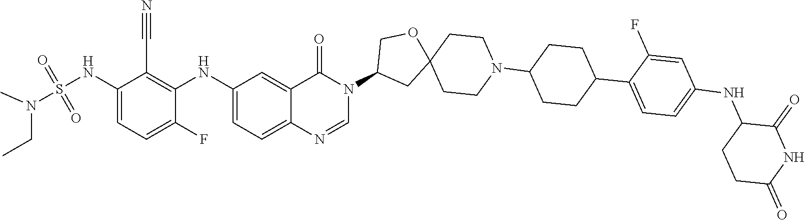 [SMILES: CCN(C)S(=O)(=O)Nc1ccc(F)c(Nc2ccc3ncn([C@H]4COC5(CCN(C6CCC(c7ccc(NC8CCC(=O)NC8=O)cc7F)CC6)CC5)C4)c(=O)c3c2)c1C#N] Compound 57 Stereoisomer 2 | Compound 57 |
| 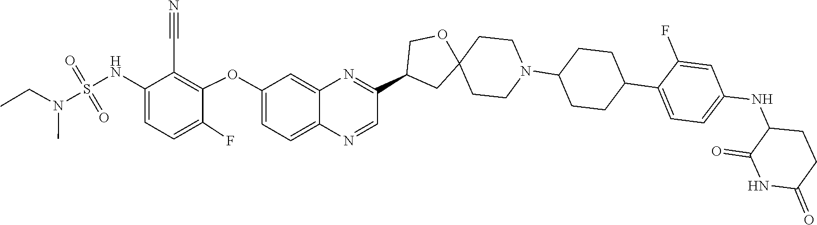 [SMILES: CCN(C)S(=O)(=O)Nc1ccc(F)c(Oc2ccc3ncc([C@H]4COC5(CCN(C6CCC(c7ccc(NC8CCC(=O)NC8=O)cc7F)CC6)CC5)C4)nc3c2)c1C#N] Compound 58 Stereoisomer 1 | Compound 58 |
| 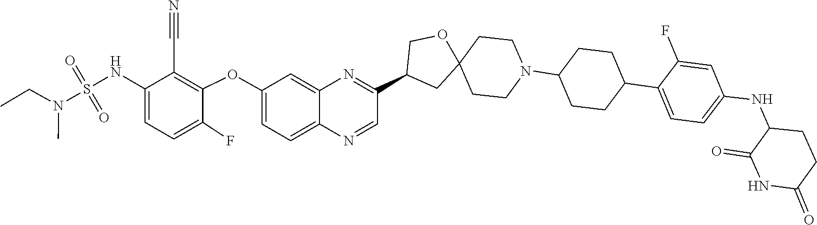 [SMILES: CCN(C)S(=O)(=O)Nc1ccc(F)c(Oc2ccc3ncc([C@H]4COC5(CCN(C6CCC(c7ccc(NC8CCC(=O)NC8=O)cc7F)CC6)CC5)C4)nc3c2)c1C#N] Compound 59 Stereoisomer 2 | Compound 59 |
| 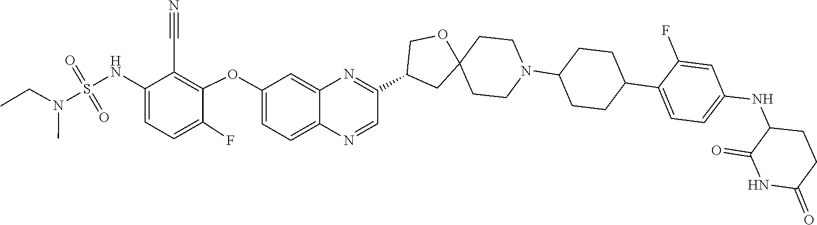 [SMILES: CCN(C)S(=O)(=O)Nc1ccc(F)c(Oc2ccc3ncc([C@@H]4COC5(CCN(C6CCC(c7ccc(NC8CCC(=O)NC8=O)cc7F)CC6)CC5)C4)nc3c2)c1C#N] Compound 60 Stereoisomer 1 | Compound 60 |
| 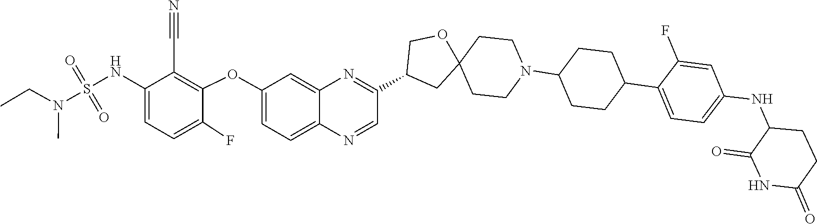 [SMILES: CCN(C)S(=O)(=O)Nc1ccc(F)c(Oc2ccc3ncc([C@@H]4COC5(CCN(C6CCC(c7ccc(NC8CCC(=O)NC8=O)cc7F)CC6)CC5)C4)nc3c2)c1C#N] Compound 61 Stereoisomer 2 | Compound 61 |
| 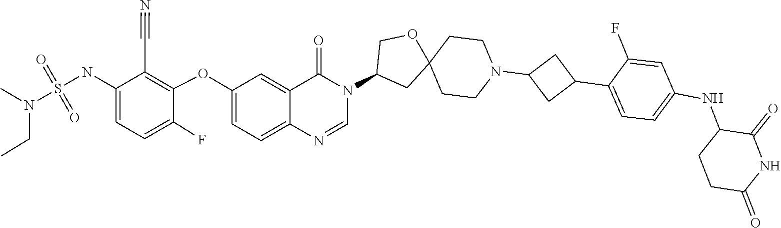 Compound 62 Stereoisomer 1 | Compound 62 |
| 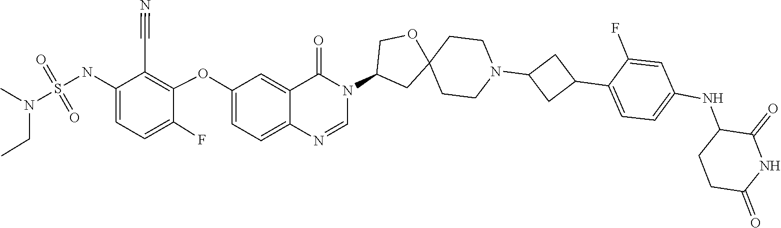 Compound 63 Stereoisomer 2 | Compound 63 |
| 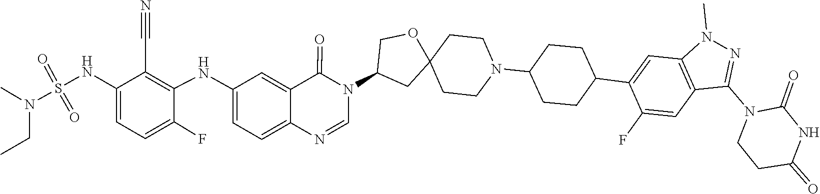 [SMILES: CCN(C)S(=O)(=O)Nc1ccc(F)c(Nc2ccc3ncn([C@H]4COC5(CCN(C6CCC(c7cc8c(cc7F)c(N7CCC(=O)NC7=O)nn8C)CC6)CC5)C4)c(=O)c3c2)c1C#N] Compound 64 Stereoisomer 1 | Compound 64 |
| 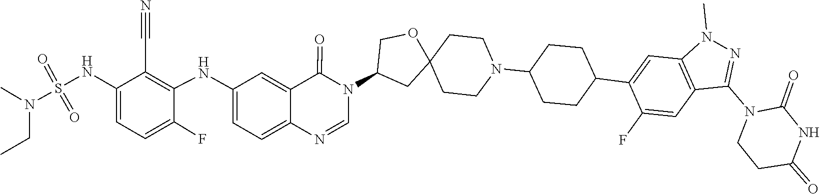 [SMILES: CCN(C)S(=O)(=O)Nc1ccc(F)c(Nc2ccc3ncn([C@H]4COC5(CCN(C6CCC(c7cc8c(cc7F)c(N7CCC(=O)NC7=O)nn8C)CC6)CC5)C4)c(=O)c3c2)c1C#N] Compound 65 Stereoisomer 2 | Compound 65 |
| 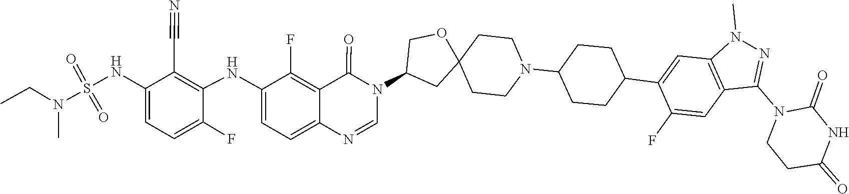 [SMILES: CCN(C)S(=O)(=O)Nc1ccc(F)c(Nc2ccc3ncn([C@H]4COC5(CCN(C6CCC(c7cc8c(cc7F)c(N7CCC(=O)NC7=O)nn8C)CC6)CC5)C4)c(=O)c3c2F)c1C#N] Compound 66 Stereoisomer 1 | Compound 66 |
| 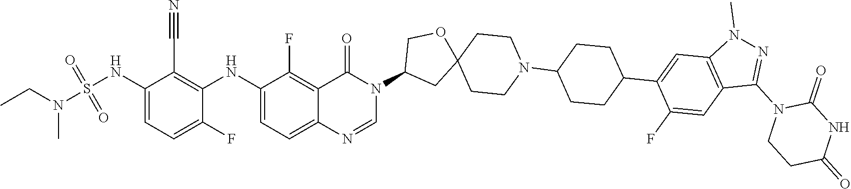 [SMILES: CCN(C)S(=O)(=O)Nc1ccc(F)c(Nc2ccc3ncn([C@H]4COC5(CCN(C6CCC(c7cc8c(cc7F)c(N7CCC(=O)NC7=O)nn8C)CC6)CC5)C4)c(=O)c3c2F)c1C#N] Compound 67 Stereoisomer 2 | Compound 67 |
| 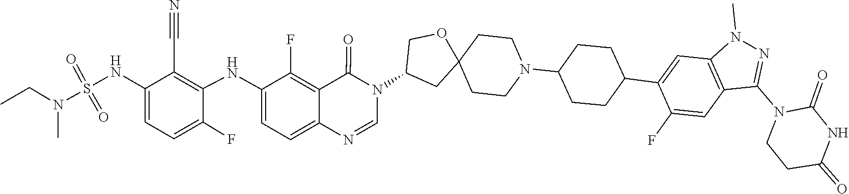 [SMILES: CCN(C)S(=O)(=O)Nc1ccc(F)c(Nc2ccc3ncn([C@@H]4COC5(CCN(C6CCC(c7cc8c(cc7F)c(N7CCC(=O)NC7=O)nn8C)CC6)CC5)C4)c(=O)c3c2F)c1C#N] Compound 68 Stereoisomer 1 | Compound 68 |
| 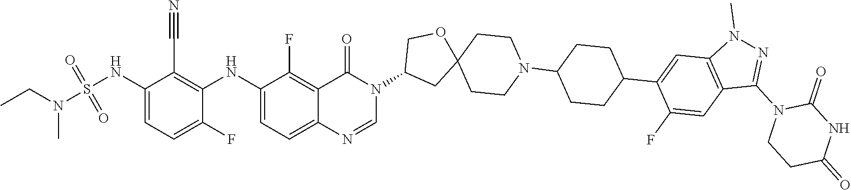 [SMILES: CCN(C)S(=O)(=O)Nc1ccc(F)c(Nc2ccc3ncn([C@@H]4COC5(CCN(C6CCC(c7cc8c(cc7F)c(N7CCC(=O)NC7=O)nn8C)CC6)CC5)C4)c(=O)c3c2F)c1C#N] Compound 69 Stereoisomer 2 | Compound 69 |
| 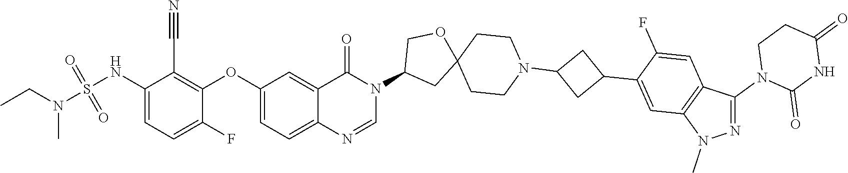 [SMILES: CCN(C)S(=O)(=O)Nc1ccc(F)c(Oc2ccc3ncn([C@H]4COC5(CCN(C6CC(c7cc8c(cc7F)c(N7CCC(=O)NC7=O)nn8C)C6)CC5)C4)c(=O)c3c2)c1C#N] Compound 70 Stereoisomer 1 | Compound 70 |
| 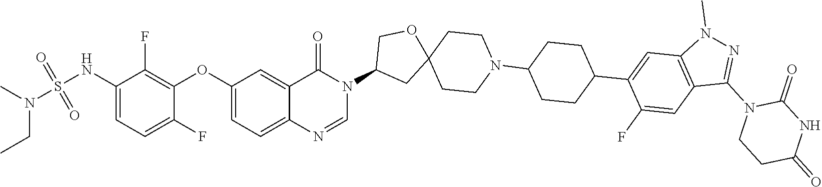 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1F] Compound 71 Stereoisomer 1 | Compound 71 |
| 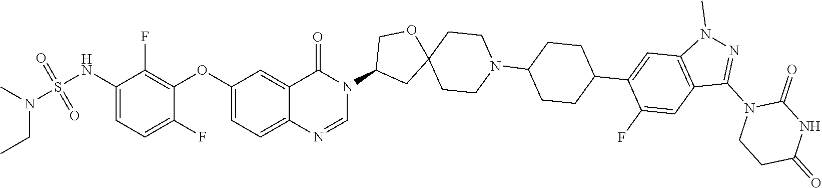 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1F] Compound 72 Stereoisomer 2 | Compound 72 |
| 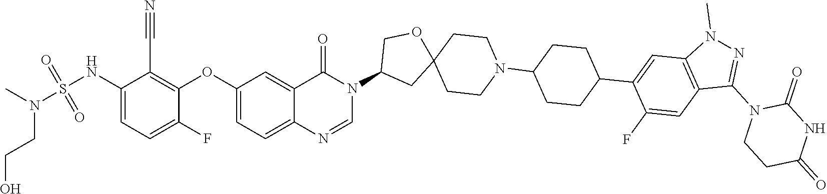 [SMILES: CN(CCO)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1C#N] Compound 73 Stereoisomer 1 | Compound 73 |
| 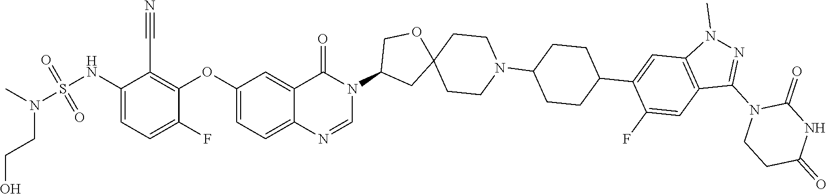 [SMILES: CN(CCO)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1C#N] Compound 74 Stereoisomer 2 | Compound 74 |
| 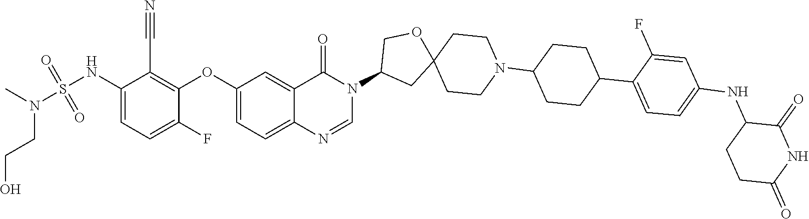 [SMILES: CN(CCO)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2)c1C#N] Compound 75 Stereoisomer 1 | Compound 75 |
| 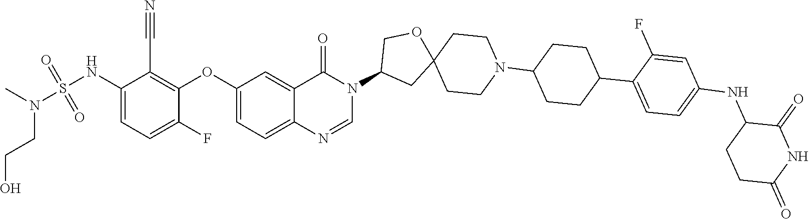 [SMILES: CN(CCO)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2)c1C#N] Compound 76 Stereoisomer 2 | Compound 76 |
| 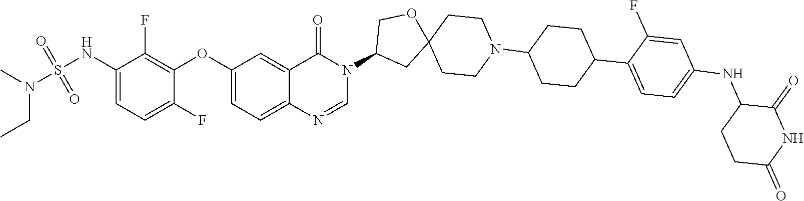 [SMILES: CCN(C)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2)c1F] Compound 77 Stereoisomer 1 | Compound 77 |
| 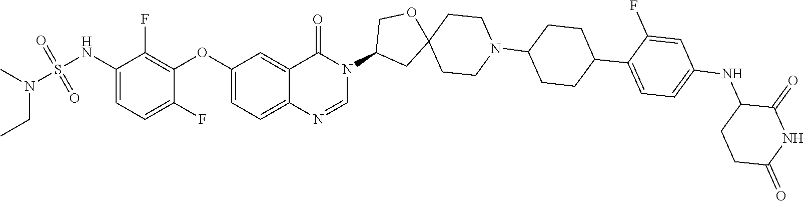 [SMILES: CCN(C)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2)c1F] Compound 78 Stereoisomer 2 | Compound 78 |
| 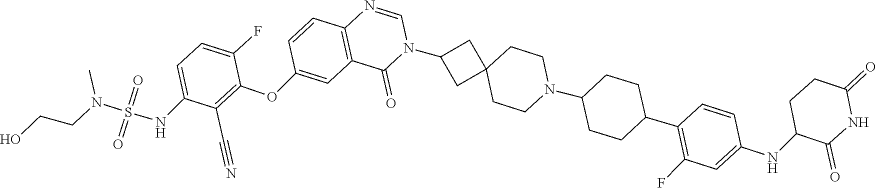 [SMILES: CN(CCO)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1C#N] Compound 79 Stereoisomer 1 | Compound 79 |
| 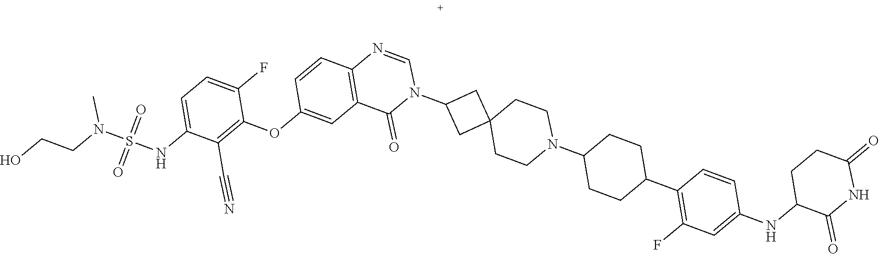 [SMILES: CN(CCO)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1C#N] Compound 80 Stereoisomer 2 | Compound 80 |
| 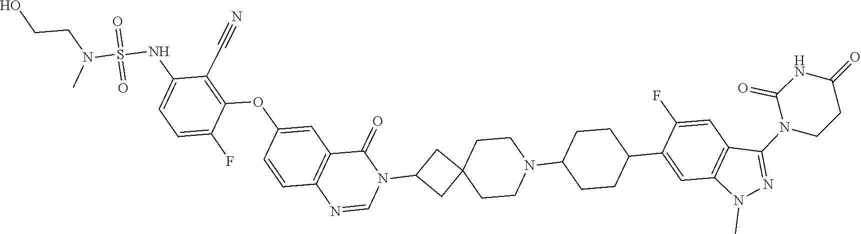 [SMILES: CN(CCO)S(=O)(=O)Nc1ccc(F)c(Oc2ccc3ncn(C4CC5(CCN(C6CCC(c7cc8c(cc7F)c(N7CCC(=O)NC7=O)nn8C)CC6)CC5)C4)c(=O)c3c2)c1C#N] Compound 81 Stereoisomer 1 | Compound 81 |
| 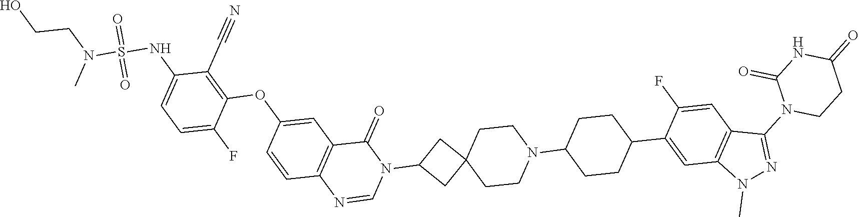 [SMILES: CN(CCO)S(=O)(=O)Nc1ccc(F)c(Oc2ccc3ncn(C4CC5(CCN(C6CCC(c7cc8c(cc7F)c(N7CCC(=O)NC7=O)nn8C)CC6)CC5)C4)c(=O)c3c2)c1C#N] Compound 82 Stereoisomer 2 | Compound 82 |
| 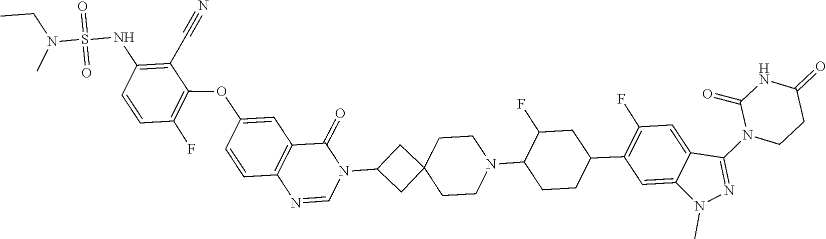 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N] Compound 83 Stereoisomer 1 | Compound 83 |
| 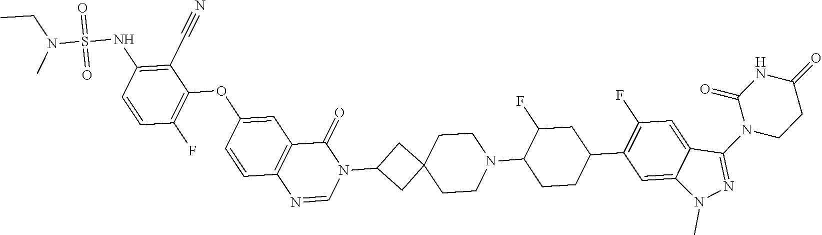 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N] Compound 84 Stereoisomer 2 | Compound 84 |
| 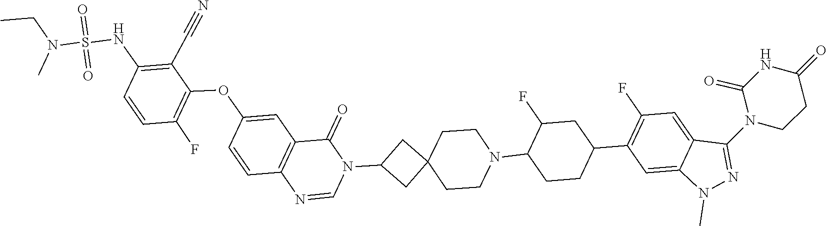 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N] Compound 85 Stereoisomer 1 | Compound 85 |
| 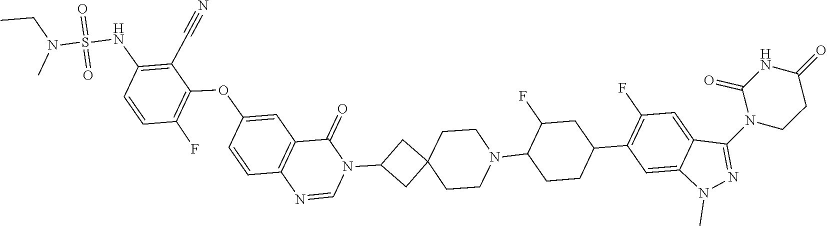 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N] Compound 86 Stereoisomer 2 | Compound 86 |
| 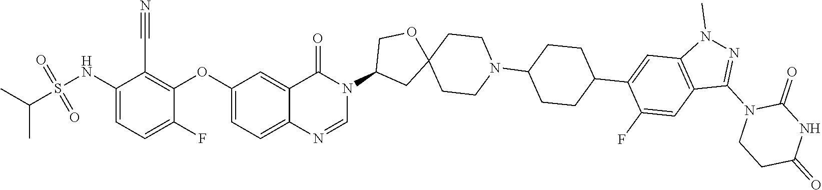 [SMILES: CC(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1C#N] Compound 87 Stereoisomer 1 | Compound 87 |
| 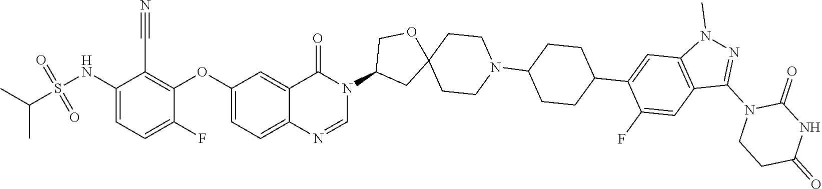 [SMILES: CC(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1C#N] Compound 88 Stereoisomer 2 | Compound 88 |
| 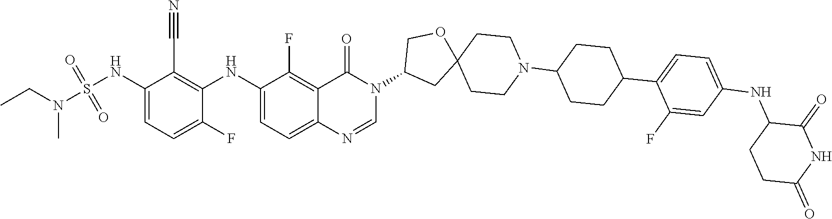 [SMILES: CCN(C)S(=O)(=O)Nc1ccc(F)c(Nc2ccc3ncn([C@@H]4COC5(CCN(C6CCC(c7ccc(NC8CCC(=O)NC8=O)cc7F)CC6)CC5)C4)c(=O)c3c2F)c1C#N] Compound 89 Stereoisomer 1 | Compound 89 |
| 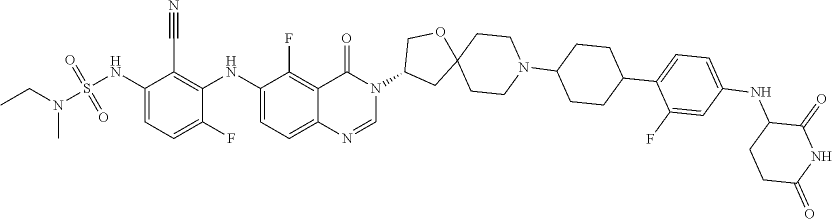 [SMILES: CCN(C)S(=O)(=O)Nc1ccc(F)c(Nc2ccc3ncn([C@@H]4COC5(CCN(C6CCC(c7ccc(NC8CCC(=O)NC8=O)cc7F)CC6)CC5)C4)c(=O)c3c2F)c1C#N] Compound 90 Stereoisomer 2 | Compound 90 |
| 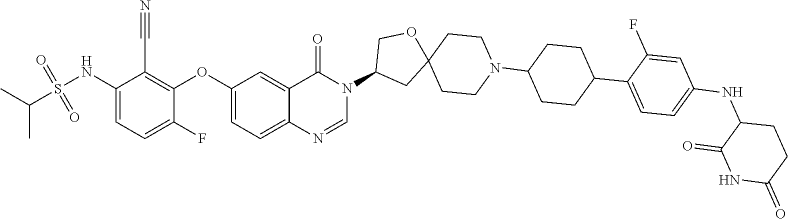 [SMILES: CC(C)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2)c1C#N] Compound 91 Stereoisomer 1 | Compound 91 |
| 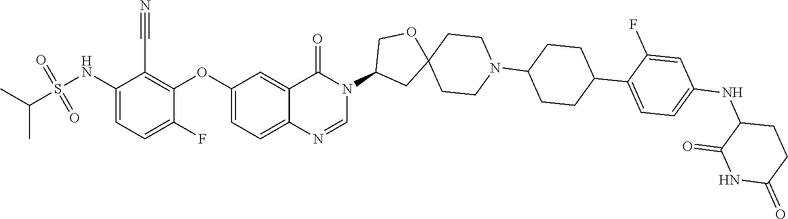 [SMILES: CC(C)S(=O)(=O)Nc1ccc(F)c(Oc2ccc3ncn([C@H]4COC5(CCN(C6CCC(c7ccc(NC8CCC(=O)NC8=O)cc7F)CC6)CC5)C4)c(=O)c3c2)c1C#N] Compound 92 Stereoisomer 2 | Compound 92 |
| 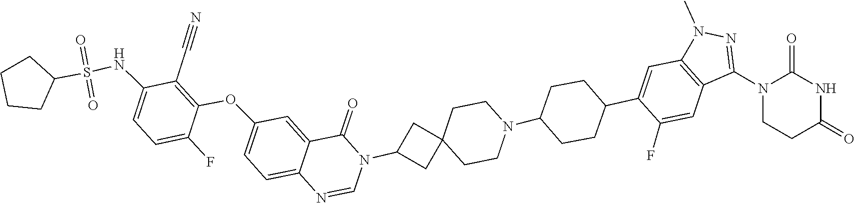 [SMILES: Cn1nc(N2CCC(=O)NC2=O)c2cc(F)c(C3CCC(N4CCC5(CC4)CC(n4cnc6ccc(Oc7c(F)ccc(NS(=O)(=O)C8CCCC8)c7C#N)cc6c4=O)C5)CC3)cc21] Compound 93 Stereoisomer 1 | Compound 93 |
| 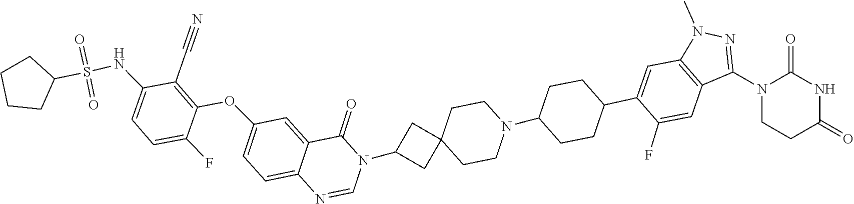 [SMILES: Cn1nc(N2CCC(=O)NC2=O)c2cc(F)c(C3CCC(N4CCC5(CC4)CC(n4cnc6ccc(Oc7c(F)ccc(NS(=O)(=O)C8CCCC8)c7C#N)cc6c4=O)C5)CC3)cc21] Compound 94 Stereoisomer 2 | Compound 94 |
| 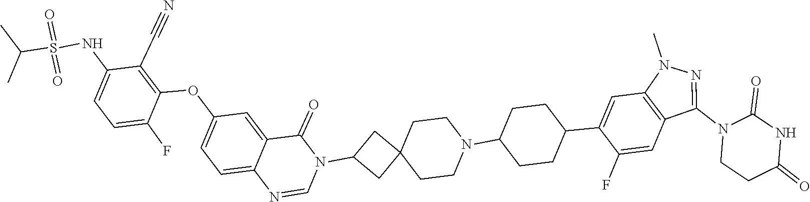 [SMILES: CC(C)S(=O)(=O)Nc1ccc(F)c(Oc2ccc3ncn(C4CC5(CCN(C6CCC(c7cc8c(cc7F)c(N7CCC(=O)NC7=O)nn8C)CC6)CC5)C4)c(=O)c3c2)c1C#N] Compound 95 Stereoisomer 1 | Compound 95 |
| 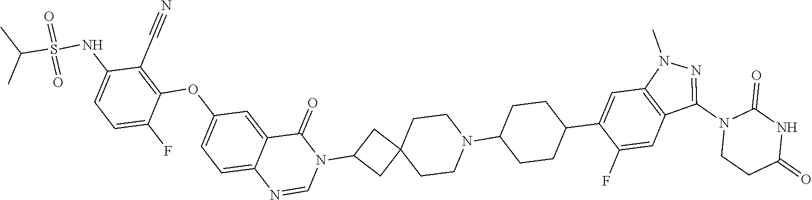 [SMILES: CC(C)S(=O)(=O)Nc1ccc(F)c(Oc2ccc3ncn(C4CC5(CCN(C6CCC(c7cc8c(cc7F)c(N7CCC(=O)NC7=O)nn8C)CC6)CC5)C4)c(=O)c3c2)c1C#N] Compound 96 Stereoisomer 2 | Compound 96 |
| 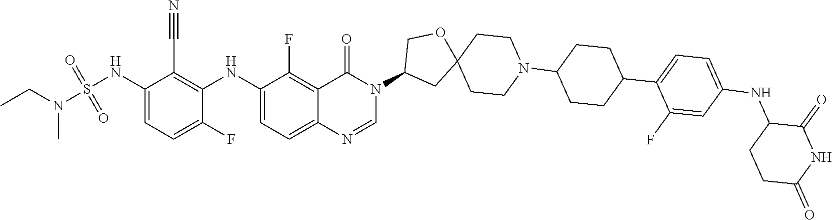 [SMILES: CCN(C)S(=O)(=O)Nc1ccc(F)c(Nc2ccc3ncn([C@H]4COC5(CCN(C6CCC(c7ccc(NC8CCC(=O)NC8=O)cc7F)CC6)CC5)C4)c(=O)c3c2F)c1C#N] Compound 97 Stereoisomer 1 | Compound 97 |
| 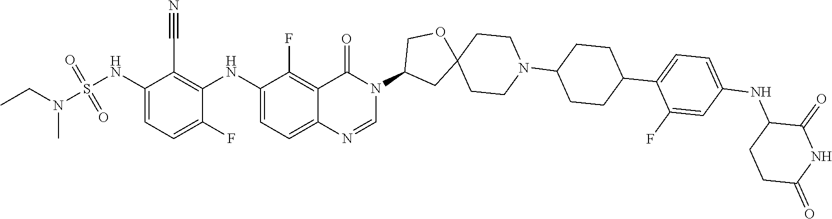 [SMILES: CCN(C)S(=O)(=O)Nc1ccc(F)c(Nc2ccc3ncn([C@H]4COC5(CCN(C6CCC(c7ccc(NC8CCC(=O)NC8=O)cc7F)CC6)CC5)C4)c(=O)c3c2F)c1C#N] Compound 98 Stereoisomer 2 | Compound 998 |
| 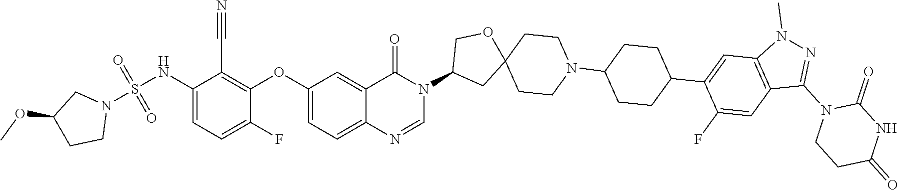 [SMILES: CO[C@@H]1CCN(S(=O)(=O)Nc2ccc(F)c(Oc3ccc4ncn([C@H]5COC6(CCN(C7CCC(c8cc9c(cc8F)c(N8CCC(=O)NC8=O)nn9C)CC7)CC6)C5)c(=O)c4c3)c2C#N)C1] Compound 99 Stereoisomer 1 | Compound 99 |
| 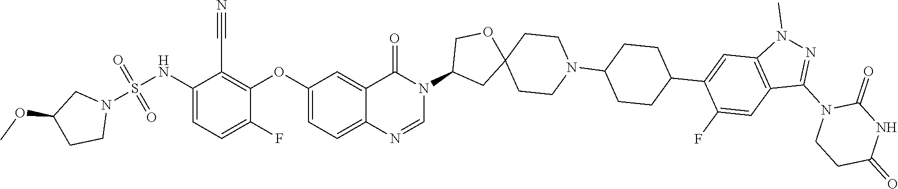 [SMILES: CO[C@@H]1CCN(S(=O)(=O)Nc2ccc(F)c(Oc3ccc4ncn([C@H]5COC6(CCN(C7CCC(c8cc9c(cc8F)c(N8CCC(=O)NC8=O)nn9C)CC7)CC6)C5)c(=O)c4c3)c2C#N)C1] Compound 100 Stereoisomer 2 | Compound 100 |
| 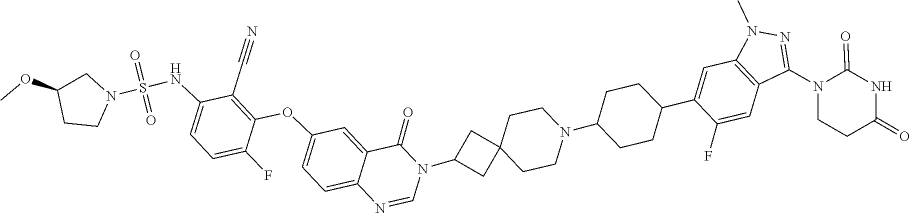 [SMILES: CO[C@@H]1CCN(S(=O)(=O)Nc2ccc(F)c(Oc3ccc4ncn(C5CC6(CCN(C7CCC(c8cc9c(cc8F)c(N8CCC(=O)NC8=O)nn9C)CC7)CC6)C5)c(=O)c4c3)c2C#N)C1] Compound 101 Stereoisomer 1 | Compound 101 |
| 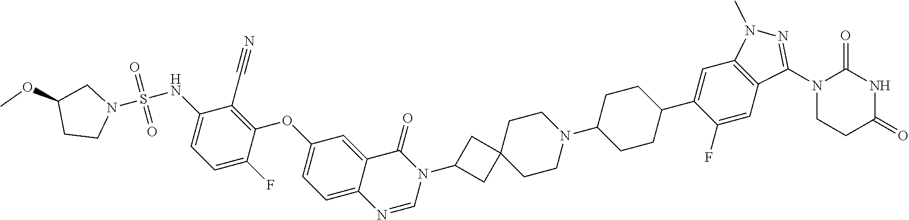 [SMILES: CO[C@@H]1CCN(S(=O)(=O)Nc2ccc(F)c(Oc3ccc4ncn(C5CC6(CCN(C7CCC(c8cc9c(cc8F)c(N8CCC(=O)NC8=O)nn9C)CC7)CC6)C5)c(=O)c4c3)c2C#N)C1] Compound 102 Stereoisomer 2 | Compound 102 |
| 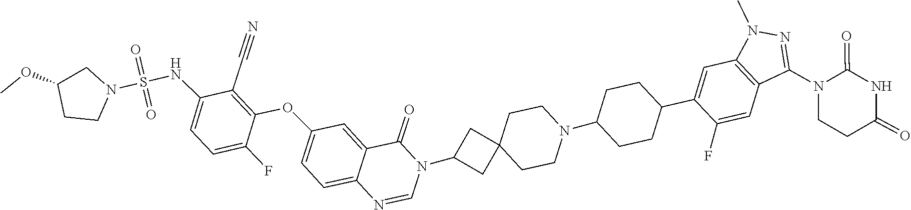 [SMILES: CO[C@H]1CCN(S(=O)(=O)Nc2ccc(F)c(Oc3ccc4ncn(C5CC6(CCN(C7CCC(c8cc9c(cc8F)c(N8CCC(=O)NC8=O)nn9C)CC7)CC6)C5)c(=O)c4c3)c2C#N)C1] Compound 103 Stereoisomer 1 | Compound 103 |
| 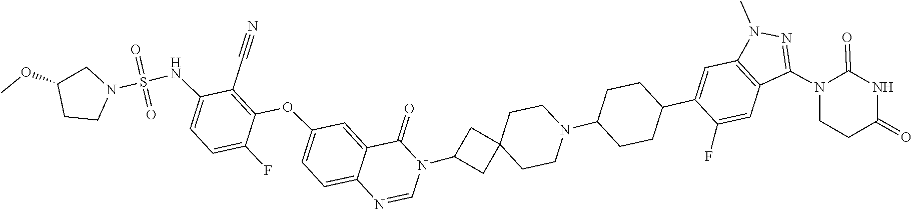 [SMILES: CO[C@H]1CCN(S(=O)(=O)Nc2ccc(F)c(Oc3ccc4ncn(C5CC6(CCN(C7CCC(c8cc9c(cc8F)c(N8CCC(=O)NC8=O)nn9C)CC7)CC6)C5)c(=O)c4c3)c2C#N)C1] Compound 104 Stereoisomer 2 | Compound 104 |
| 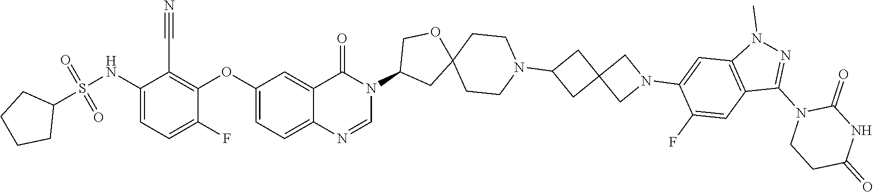 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CCCC9)c8C#N)cc7c5=O)CO6)C4)C3)cc21] Compound 105 | Compound 105 |
| 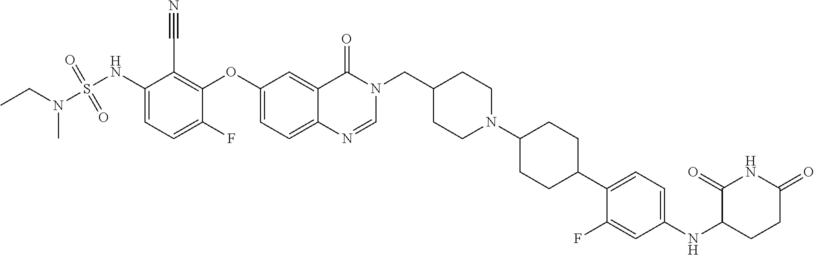 [SMILES: CCN(C)S(=O)(=O)Nc1ccc(F)c(Oc2ccc3ncn(CC4CCN(C5CCC(c6ccc(NC7CCC(=O)NC7=O)cc6F)CC5)CC4)c(=O)c3c2)c1C#N] Compound 106 Stereoisomer 1 | Compound 106 |
| 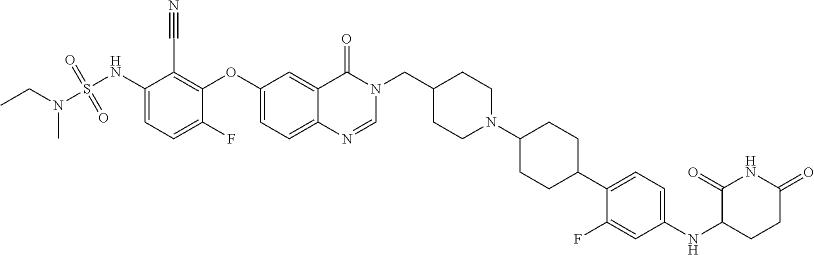 [SMILES: CCN(C)S(=O)(=O)Nc1ccc(F)c(Oc2ccc3ncn(CC4CCN(C5CCC(c6ccc(NC7CCC(=O)NC7=O)cc6F)CC5)CC4)c(=O)c3c2)c1C#N] Compound 107 Stereoisomer 2 | Compound 107 |
| 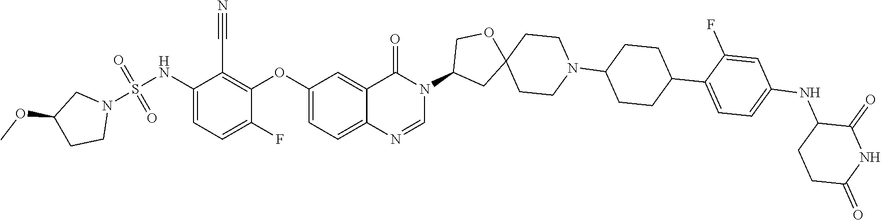 [SMILES: CO[C@@H]1CCN(S(=O)(=O)Nc2ccc(F)c(Oc3ccc4ncn([C@H]5COC6(CCN(C7CCC(c8ccc(NC9CCC(=O)NC9=O)cc8F)CC7)CC6)C5)c(=O)c4c3)c2C#N)C1] Compound 108 Stereoisomer 1 | Compound 108 |
| 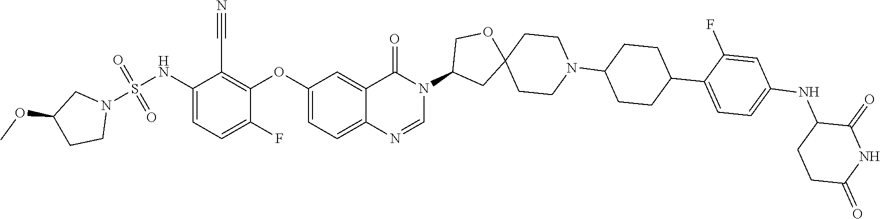 [SMILES: CO[C@@H]1CCN(S(=O)(=O)Nc2ccc(F)c(Oc3ccc4ncn([C@H]5COC6(CCN(C7CCC(c8ccc(NC9CCC(=O)NC9=O)cc8F)CC7)CC6)C5)c(=O)c4c3)c2C#N)C1] Compound 109 Stereoisomer 2 | Compound 109 |
| 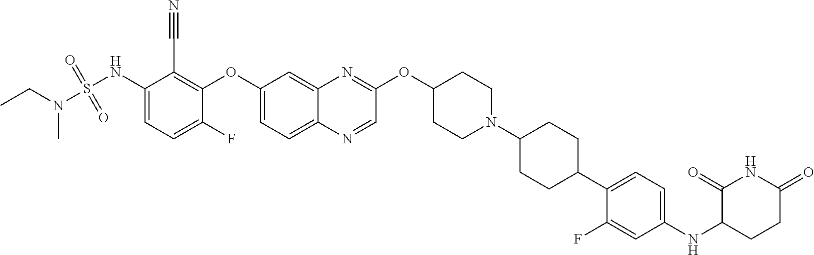 [SMILES: CCN(C)S(=O)(=O)Nc1ccc(F)c(Oc2ccc3ncc(OC4CCN(C5CCC(c6ccc(NC7CCC(=O)NC7=O)cc6F)CC5)CC4)nc3c2)c1C#N] Compound 110 Stereoisomer 1 | Compound 110 |
| 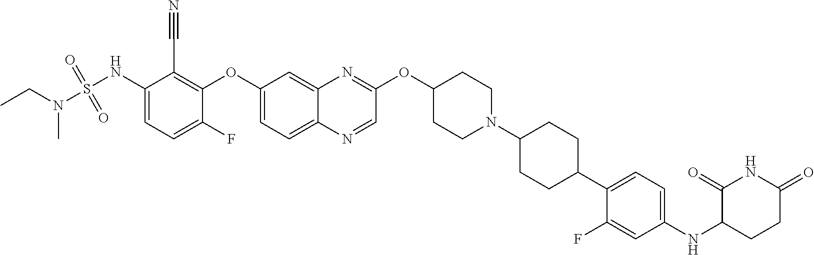 [SMILES: CCN(C)S(=O)(=O)Nc1ccc(F)c(Oc2ccc3ncc(OC4CCN(C5CCC(c6ccc(NC7CCC(=O)NC7=O)cc6F)CC5)CC4)nc3c2)c1C#N] Compound 111 Stereoisomer 2 | Compound 111 |
| 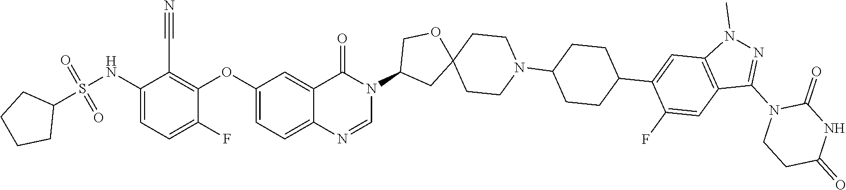 [SMILES: Cn1nc(N2CCC(=O)NC2=O)c2cc(F)c(C3CCC(N4CCC5(CC4)C[C@@H](n4cnc6ccc(Oc7c(F)ccc(NS(=O)(=O)C8CCCC8)c7C#N)cc6c4=O)CO5)CC3)cc21] Compound 112 Stereoisomer 1 | Compound 112 |
| 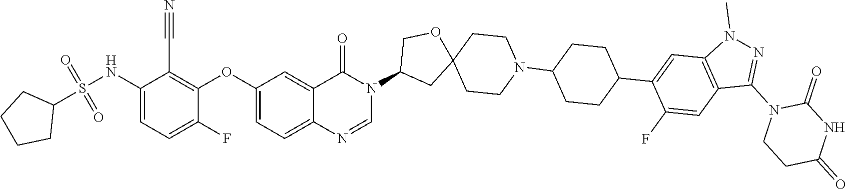 [SMILES: Cn1nc(N2CCC(=O)NC2=O)c2cc(F)c(C3CCC(N4CCC5(CC4)C[C@@H](n4cnc6ccc(Oc7c(F)ccc(NS(=O)(=O)C8CCCC8)c7C#N)cc6c4=O)CO5)CC3)cc21] Compound 113 Stereoisomer 2 | Compound 113 |
| 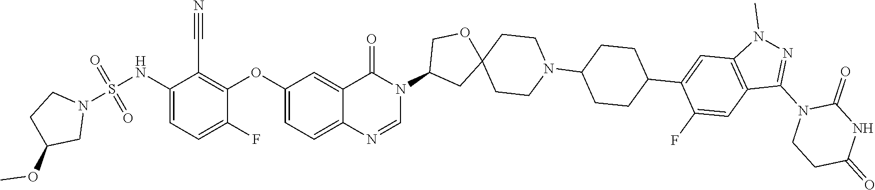 [SMILES: CO[C@H]1CCN(S(=O)(=O)Nc2ccc(F)c(Oc3ccc4ncn([C@H]5COC6(CCN(C7CCC(c8cc9c(cc8F)c(N8CCC(=O)NC8=O)nn9C)CC7)CC6)C5)c(=O)c4c3)c2C#N)C1] Compound 114 Stereoisomer 1 | Compound 114 |
| 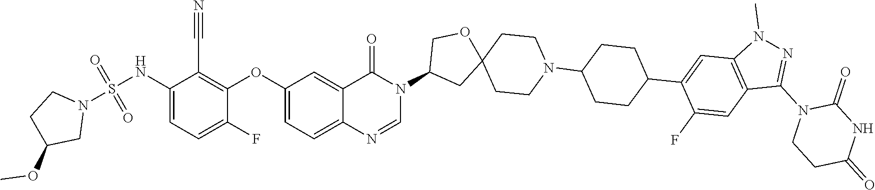 [SMILES: CO[C@H]1CCN(S(=O)(=O)Nc2ccc(F)c(Oc3ccc4ncn([C@H]5COC6(CCN(C7CCC(c8cc9c(cc8F)c(N8CCC(=O)NC8=O)nn9C)CC7)CC6)C5)c(=O)c4c3)c2C#N)C1] Compound 115 Stereoisomer 2 | Compound 115 |
| 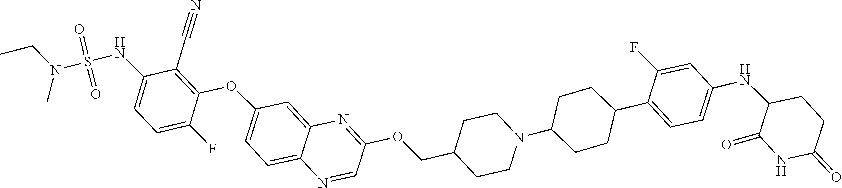 [SMILES: CCN(C)S(=O)(=O)Nc1ccc(F)c(Oc2ccc3ncc(OCC4CCN(C5CCC(c6ccc(NC7CCC(=O)NC7=O)cc6F)CC5)CC4)nc3c2)c1C#N] Compound 116 Stereoisomer 1 | Compound 116 |
| 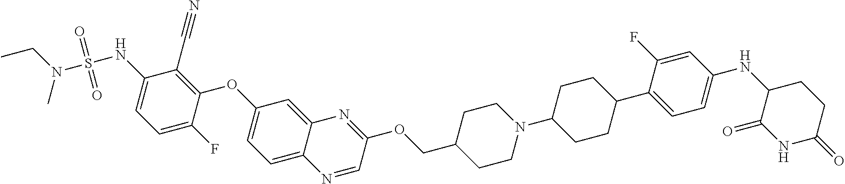 [SMILES: CCN(C)S(=O)(=O)Nc1ccc(F)c(Oc2ccc3ncc(OCC4CCN(C5CCC(c6ccc(NC7CCC(=O)NC7=O)cc6F)CC5)CC4)nc3c2)c1C#N] Compound 117 Stereoisomer 2 | Compound 117 |
| 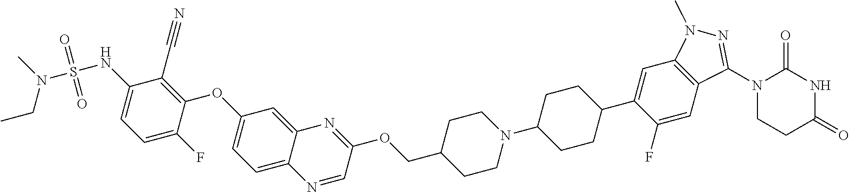 [SMILES: CCN(C)S(=O)(=O)Nc1ccc(F)c(Oc2ccc3ncc(OCC4CCN(C5CCC(c6cc7c(cc6F)c(N6CCC(=O)NC6=O)nn7C)CC5)CC4)nc3c2)c1C#N] Compound 118 Stereoisomer 1 | Compound 118 |
| 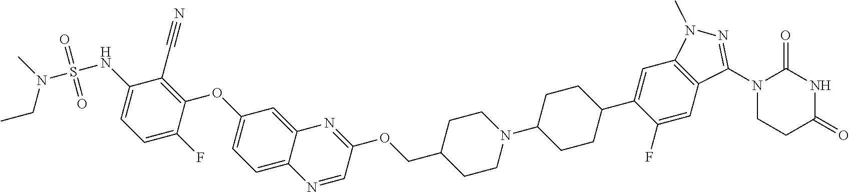 [SMILES: CCN(C)S(=O)(=O)Nc1ccc(F)c(Oc2ccc3ncc(OCC4CCN(C5CCC(c6cc7c(cc6F)c(N6CCC(=O)NC6=O)nn7C)CC5)CC4)nc3c2)c1C#N] Compound 119 Stereoisomer 2 | Compound 119 |
| 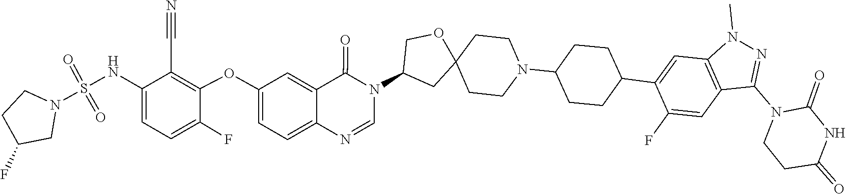 [SMILES: Cn1nc(N2CCC(=O)NC2=O)c2cc(F)c(C3CCC(N4CCC5(CC4)C[C@@H](n4cnc6ccc(Oc7c(F)ccc(NS(=O)(=O)N8CC[C@@H](F)C8)c7C#N)cc6c4=O)CO5)CC3)cc21] Compound 120 Stereoisomer 1 | Compound 120 |
| 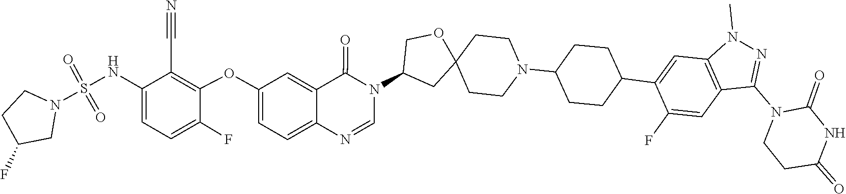 [SMILES: Cn1nc(N2CCC(=O)NC2=O)c2cc(F)c(C3CCC(N4CCC5(CC4)C[C@@H](n4cnc6ccc(Oc7c(F)ccc(NS(=O)(=O)N8CC[C@@H](F)C8)c7C#N)cc6c4=O)CO5)CC3)cc21] Compound 121 Stereoisomer 2 | Compound 121 |
| 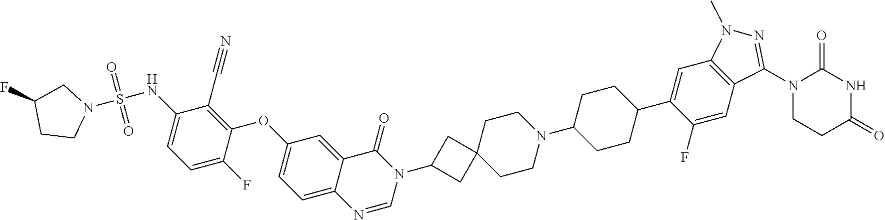 [SMILES: Cn1nc(N2CCC(=O)NC2=O)c2cc(F)c(C3CCC(N4CCC5(CC4)CC(n4cnc6ccc(Oc7c(F)ccc(NS(=O)(=O)N8CC[C@@H](F)C8)c7C#N)cc6c4=O)C5)CC3)cc21] Compound 122 Stereoisomer 1 | Compound 122 |
| 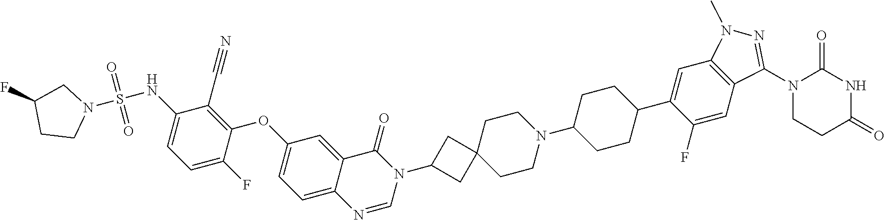 [SMILES: Cn1nc(N2CCC(=O)NC2=O)c2cc(F)c(C3CCC(N4CCC5(CC4)CC(n4cnc6ccc(Oc7c(F)ccc(NS(=O)(=O)N8CC[C@@H](F)C8)c7C#N)cc6c4=O)C5)CC3)cc21] Compound 123 Stereoisomer 2 | Compound 123 |
| 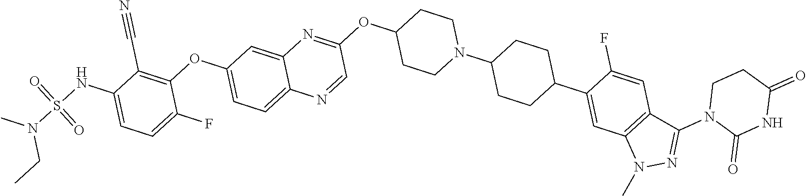 [SMILES: CCN(C)S(=O)(=O)Nc1ccc(F)c(Oc2ccc3ncc(OC4CCN(C5CCC(c6cc7c(cc6F)c(N6CCC(=O)NC6=O)nn7C)CC5)CC4)nc3c2)c1C#N] Compound 124 Stereoisomer 1 | Compound 124 |
| 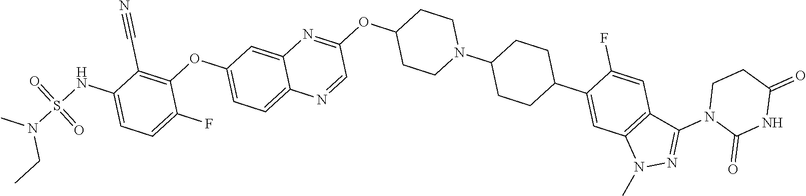 [SMILES: CCN(C)S(=O)(=O)Nc1ccc(F)c(Oc2ccc3ncc(OC4CCN(C5CCC(c6cc7c(cc6F)c(N6CCC(=O)NC6=O)nn7C)CC5)CC4)nc3c2)c1C#N] Compound 125 Stereoisomer 2 | Compound 125 |
| 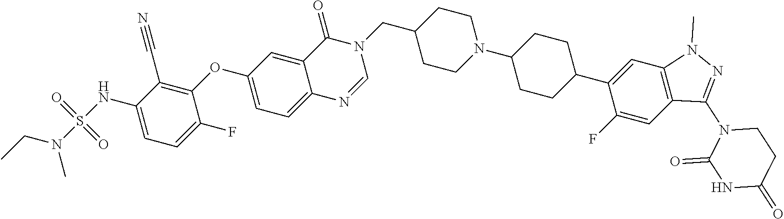 [SMILES: CCN(C)S(=O)(=O)Nc1ccc(F)c(Oc2ccc3ncn(CC4CCN(C5CCC(c6cc7c(cc6F)c(N6CCC(=O)NC6=O)nn7C)CC5)CC4)c(=O)c3c2)c1C#N] Compound 126 Stereoisomer 1 | Compound 126 |
| 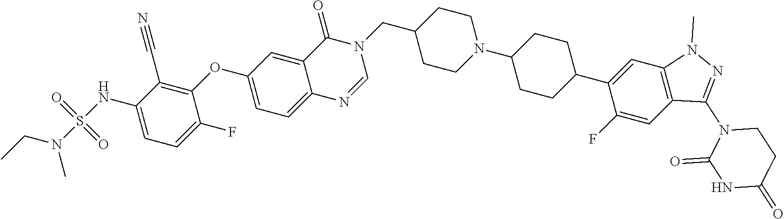 [SMILES: CCN(C)S(=O)(=O)Nc1ccc(F)c(Oc2ccc3ncn(CC4CCN(C5CCC(c6cc7c(cc6F)c(N6CCC(=O)NC6=O)nn7C)CC5)CC4)c(=O)c3c2)c1C#N] Compound 127 Stereoisomer 2 | Compound 127 |
| 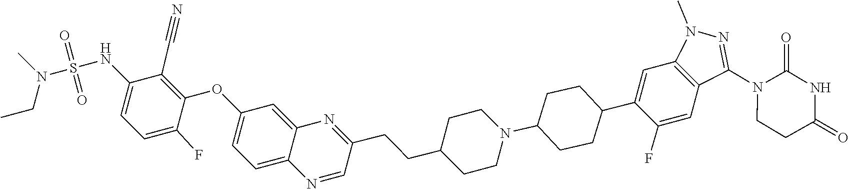 [SMILES: CCN(C)S(=O)(=O)Nc1ccc(F)c(Oc2ccc3ncc(CCC4CCN(C5CCC(c6cc7c(cc6F)c(N6CCC(=O)NC6=O)nn7C)CC5)CC4)nc3c2)c1C#N] Compound 128 Stereoisomer 1 | Compound 128 |
| 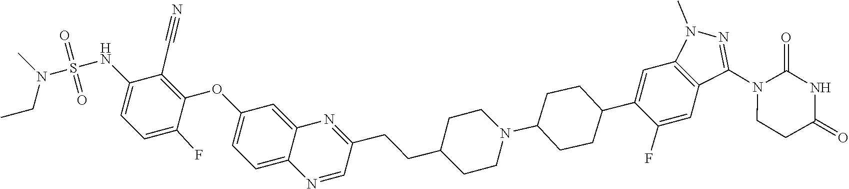 [SMILES: CCN(C)S(=O)(=O)Nc1ccc(F)c(Oc2ccc3ncc(CCC4CCN(C5CCC(c6cc7c(cc6F)c(N6CCC(=O)NC6=O)nn7C)CC5)CC4)nc3c2)c1C#N] Compound 129 Stereoisomer 2 | Compound 129 |
| 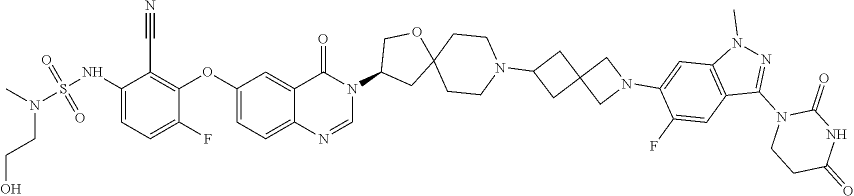 [SMILES: CN(CCO)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N] Compound 130 | Compound 130 |
| 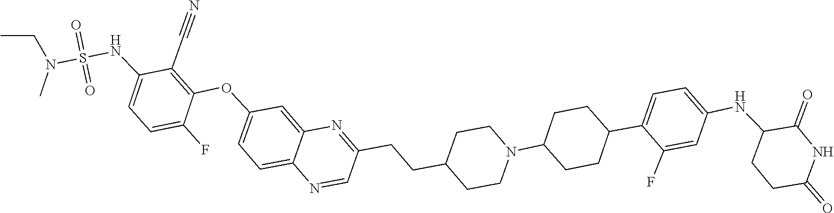 [SMILES: CCN(C)S(=O)(=O)Nc1ccc(F)c(Oc2ccc3ncc(CCC4CCN(C5CCC(c6ccc(NC7CCC(=O)NC7=O)cc6F)CC5)CC4)nc3c2)c1C#N] Compound 131 Stereoisomer 1 | Compound 131 |
| 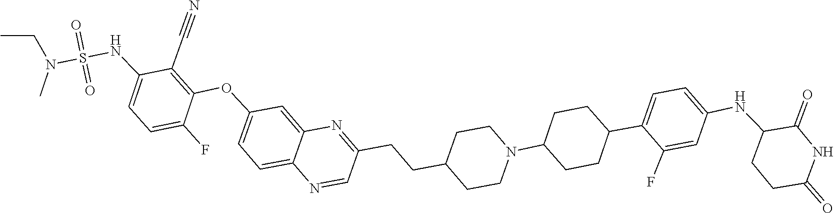 [SMILES: CCN(C)S(=O)(=O)Nc1ccc(F)c(Oc2ccc3ncc(CCC4CCN(C5CCC(c6ccc(NC7CCC(=O)NC7=O)cc6F)CC5)CC4)nc3c2)c1C#N] Compound 132 Stereoisomer 2 | Compound 132 |
| 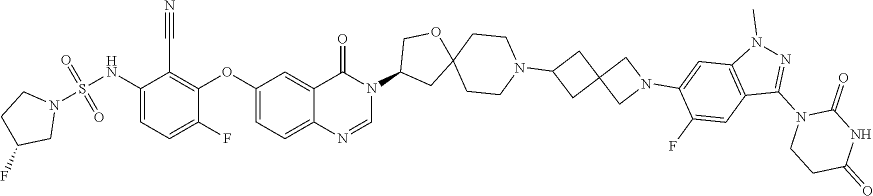 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC[C@@H](F)C9)c8C#N)cc7c5=O)CO6)C4)C3)cc21] Compound 133 | Compound 133 |
| 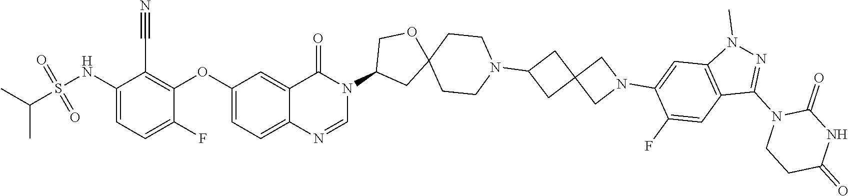 [SMILES: CC(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1C#N] Compound 134 | Compound 134 |
| 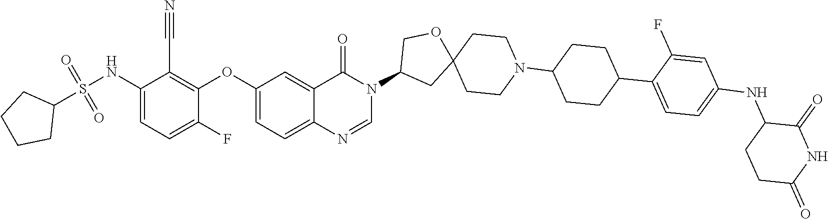 [SMILES: N#Cc1c(NS(=O)(=O)C2CCCC2)ccc(F)c1Oc1ccc2ncn([C@H]3COC4(CCN(C5CCC(c6ccc(NC7CCC(=O)NC7=O)cc6F)CC5)CC4)C3)c(=O)c2c1] Compound 135 Stereoisomer 1 | Compound 135 |
| 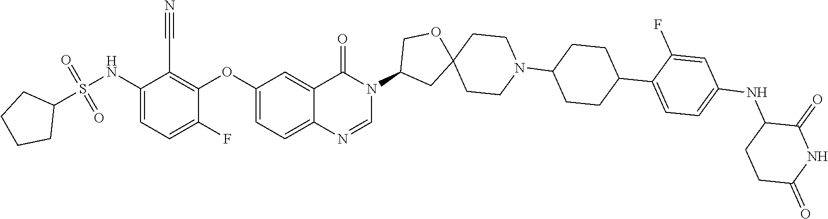 [SMILES: N#Cc1c(NS(=O)(=O)C2CCCC2)ccc(F)c1Oc1ccc2ncn([C@H]3COC4(CCN(C5CCC(c6ccc(NC7CCC(=O)NC7=O)cc6F)CC5)CC4)C3)c(=O)c2c1] Compound 136 Stereoisomer 2 | Compound 136 |
| 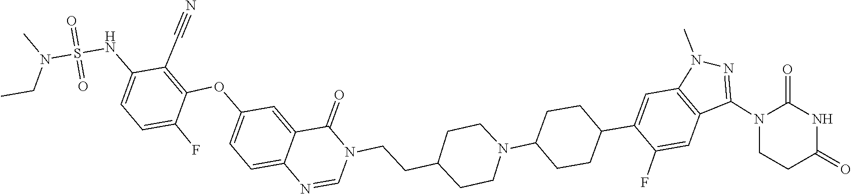 [SMILES: CCN(C)S(=O)(=O)Nc1ccc(F)c(Oc2ccc3ncn(CCC4CCN(C5CCC(c6cc7c(cc6F)c(N6CCC(=O)NC6=O)nn7C)CC5)CC4)c(=O)c3c2)c1C#N] Compound 137 Stereoisomer 1 | Compound 137 |
| 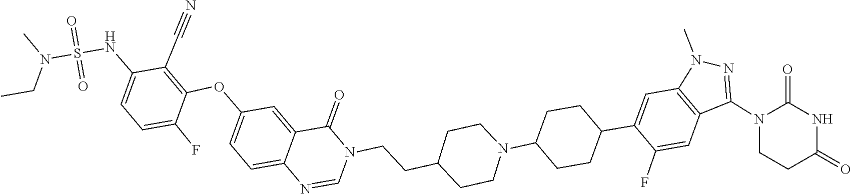 [SMILES: CCN(C)S(=O)(=O)Nc1ccc(F)c(Oc2ccc3ncn(CCC4CCN(C5CCC(c6cc7c(cc6F)c(N6CCC(=O)NC6=O)nn7C)CC5)CC4)c(=O)c3c2)c1C#N] Compound 138 Stereoisomer 2 | Compound 138 |
| 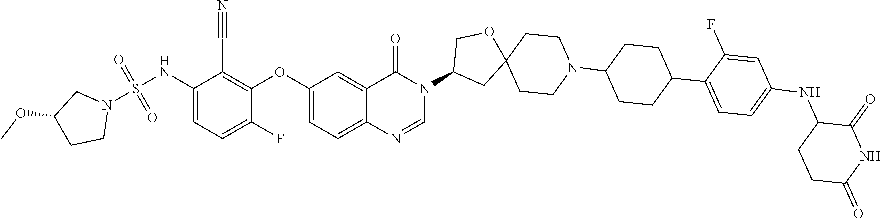 [SMILES: CO[C@H]1CCN(S(=O)(=O)Nc2ccc(F)c(Oc3ccc4ncn([C@H]5COC6(CCN(C7CCC(c8ccc(NC9CCC(=O)NC9=O)cc8F)CC7)CC6)C5)c(=O)c4c3)c2C#N)C1] Compound 139 Stereoisomer 1 | Compound 139 |
| 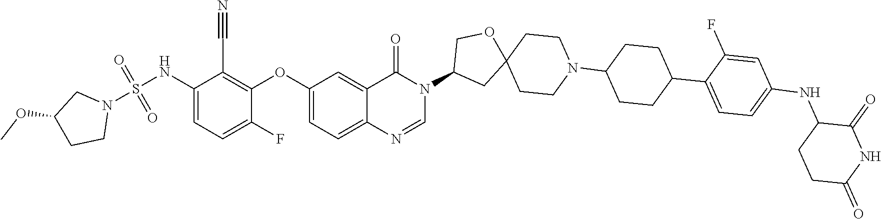 [SMILES: CO[C@H]1CCN(S(=O)(=O)Nc2ccc(F)c(Oc3ccc4ncn([C@H]5COC6(CCN(C7CCC(c8ccc(NC9CCC(=O)NC9=O)cc8F)CC7)CC6)C5)c(=O)c4c3)c2C#N)C1] Compound 140 Stereoisomer 2 | Compound 140 |
| 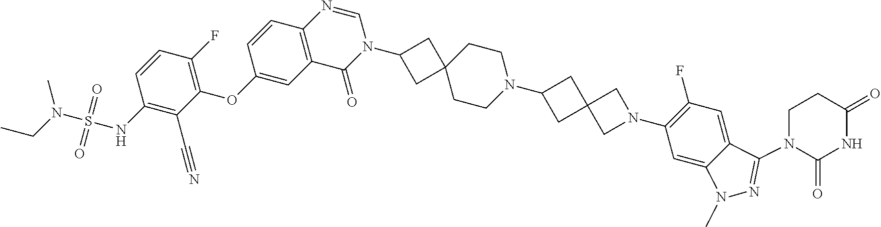 [SMILES: CCN(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1C#N] Compound 141 | Compound 141 |
| 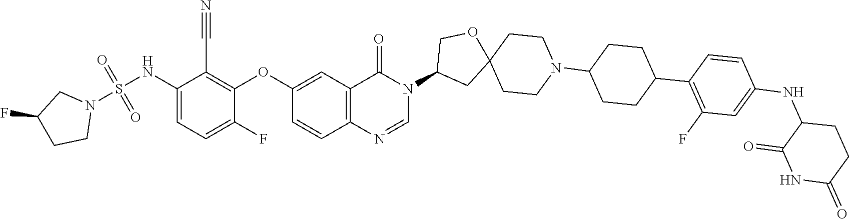 [SMILES: N#Cc1c(NS(=O)(=O)N2CC[C@@H](F)C2)ccc(F)c1Oc1ccc2ncn([C@H]3COC4(CCN(C5CCC(c6ccc(NC7CCC(=O)NC7=O)cc6F)CC5)CC4)C3)c(=O)c2c1] Compound 142 Stereoisomer 1 | Compound 142 |
| 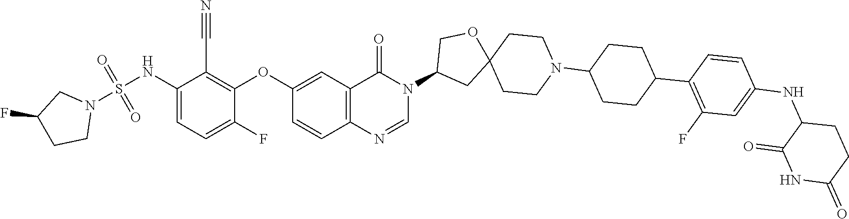 [SMILES: N#Cc1c(NS(=O)(=O)N2CC[C@@H](F)C2)ccc(F)c1Oc1ccc2ncn([C@H]3COC4(CCN(C5CCC(c6ccc(NC7CCC(=O)NC7=O)cc6F)CC5)CC4)C3)c(=O)c2c1] Compound 143 Stereoisomer 2 | Compound 143 |
| 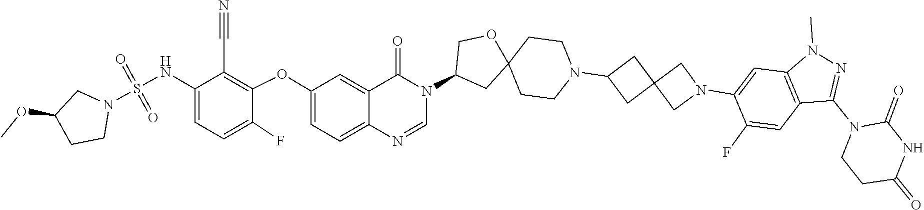 [SMILES: CO[C@@H]1CCN(S(=O)(=O)Nc2ccc(F)c(Oc3ccc4ncn([C@H]5COC6(CCN(C7CC8(C7)CN(c7cc9c(cc7F)c(N7CCC(=O)NC7=O)nn9C)C8)CC6)C5)c(=O)c4c3)c2C#N)C1] Compound 144 | Compound 144 |
| 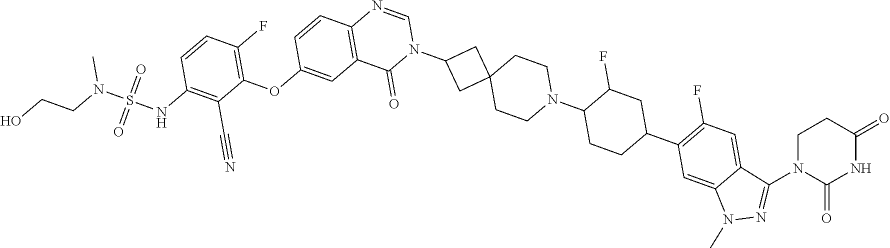 [SMILES: CN(CCO)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N] Compound 145 Stereoisomer 1 | Compound 145 |
| 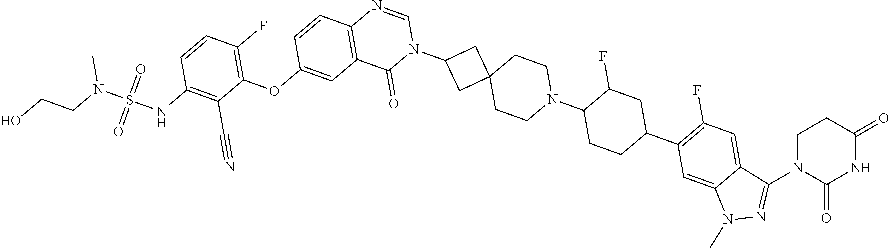 [SMILES: CN(CCO)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1C#N] Compound 146 Stereoisomer 2 | Compound 146 |
| 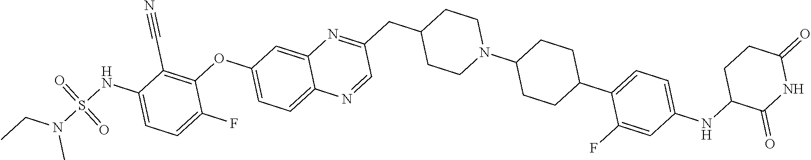 [SMILES: CCN(C)S(=O)(=O)Nc1ccc(F)c(Oc2ccc3ncc(CC4CCN(C5CCC(c6ccc(NC7CCC(=O)NC7=O)cc6F)CC5)CC4)nc3c2)c1C#N] Compound 147 Stereoisomer 1 | Compound 147 |
| 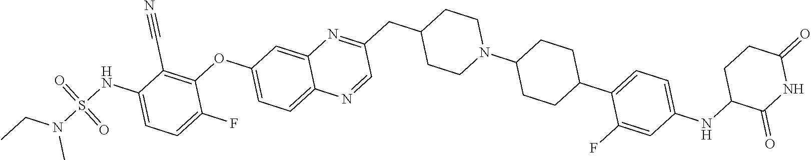 [SMILES: CCN(C)S(=O)(=O)Nc1ccc(F)c(Oc2ccc3ncc(CC4CCN(C5CCC(c6ccc(NC7CCC(=O)NC7=O)cc6F)CC5)CC4)nc3c2)c1C#N] Compound 148 Stereoisomer 2 | Compound 148 |
| 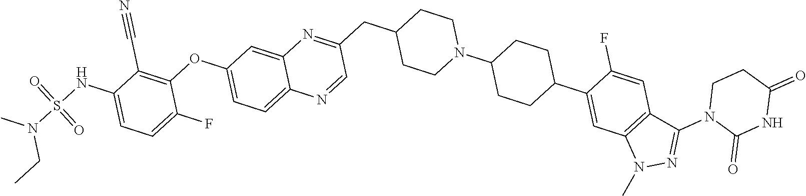 [SMILES: CCN(C)S(=O)(=O)Nc1ccc(F)c(Oc2ccc3ncc(CC4CCN(C5CCC(c6cc7c(cc6F)c(N6CCC(=O)NC6=O)nn7C)CC5)CC4)nc3c2)c1C#N] Compound 149 Stereoisomer 1 | Compound 149 |
| 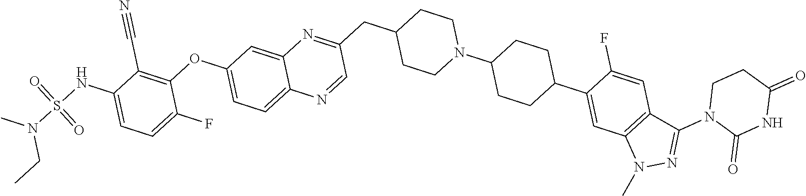 [SMILES: CCN(C)S(=O)(=O)Nc1ccc(F)c(Oc2ccc3ncc(CC4CCN(C5CCC(c6cc7c(cc6F)c(N6CCC(=O)NC6=O)nn7C)CC5)CC4)nc3c2)c1C#N] Compound 150 Stereoisomer 2 | Compound 150 |
| 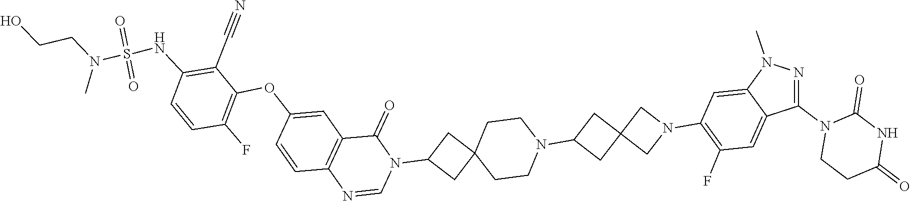 [SMILES: CN(CCO)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1C#N] Compound 151 | Compound 151 |
| 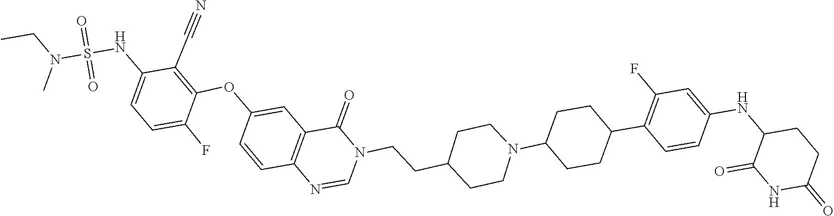 [SMILES: CCN(C)S(=O)(=O)Nc1ccc(F)c(Oc2ccc3ncn(CCC4CCN(C5CCC(c6ccc(NC7CCC(=O)NC7=O)cc6F)CC5)CC4)c(=O)c3c2)c1C#N] Compound 152 Stereoisomer 1 | Compound 152 |
| 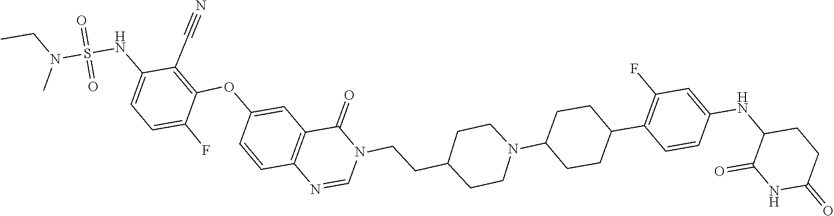 [SMILES: CCN(C)S(=O)(=O)Nc1ccc(F)c(Oc2ccc3ncn(CCC4CCN(C5CCC(c6ccc(NC7CCC(=O)NC7=O)cc6F)CC5)CC4)c(=O)c3c2)c1C#N] Compound 153 Stereoisomer 2 | Compound 153 |
| 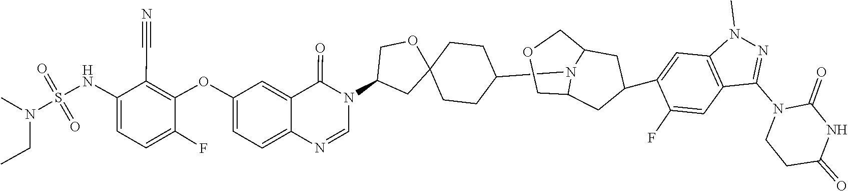 [SMILES: CCN(C)S(=O)(=O)Nc1ccc(F)c(Oc2ccc3ncn([C@H]4COC5(CCC(N6C7COCC6CC(c6cc8c(cc6F)c(N6CCC(=O)NC6=O)nn8C)C7)CC5)C4)c(=O)c3c2)c1C#N] Compound 154 Stereoisomer 1 | Compound 154 |
| 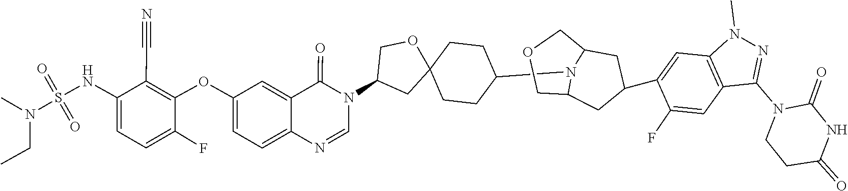 [SMILES: CCN(C)S(=O)(=O)Nc1ccc(F)c(Oc2ccc3ncn([C@H]4COC5(CCC(N6C7COCC6CC(c6cc8c(cc6F)c(N6CCC(=O)NC6=O)nn8C)C7)CC5)C4)c(=O)c3c2)c1C#N] Compound 155 Stereoisomer 2 | Compound 155 |
| 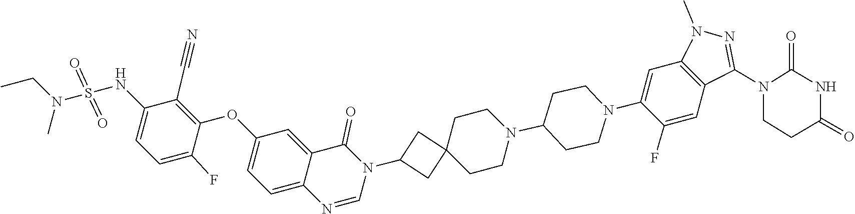 [SMILES: CCN(C)S(=O)(=O)Nc1ccc(F)c(Oc2ccc3ncn(C4CC5(CCN(C6CCN(c7cc8c(cc7F)c(N7CCC(=O)NC7=O)nn8C)CC6)CC5)C4)c(=O)c3c2)c1C#N] Compound 156 | Compound 156 |
| 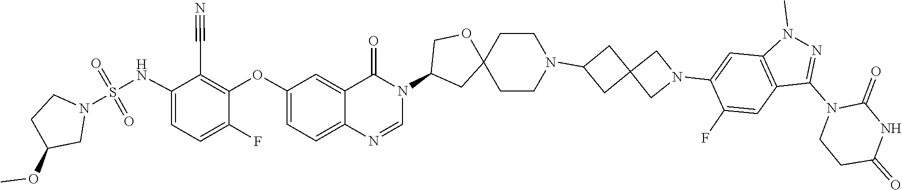 [SMILES: CO[C@H]1CCN(S(=O)(=O)Nc2ccc(F)c(Oc3ccc4ncn([C@H]5COC6(CCN(C7CC8(C7)CN(c7cc9c(cc7F)c(N7CCC(=O)NC7=O)nn9C)C8)CC6)C5)c(=O)c4c3)c2C#N)C1] Compound 157 | Compound 157 |
| 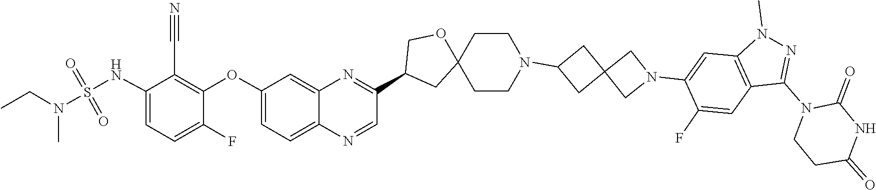 [SMILES: CCN(C)S(=O)(=O)Nc1ccc(F)c(Oc2ccc3ncc([C@H]4COC5(CCN(C6CC7(C6)CN(c6cc8c(cc6F)c(N6CCC(=O)NC6=O)nn8C)C7)CC5)C4)nc3c2)c1C#N] Compound 158 | Compound 158 |
| 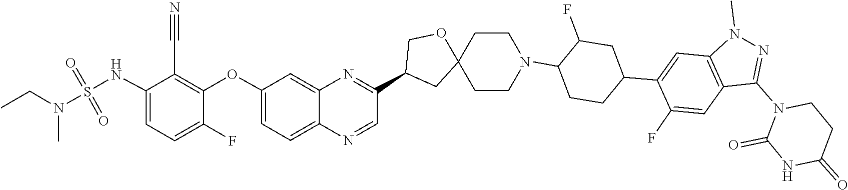 [SMILES: CCN(C)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6F)CC5)C4)nc3c2)c1C#N] Compound 159 Stereoisomer 1 | Compound 159 |
| 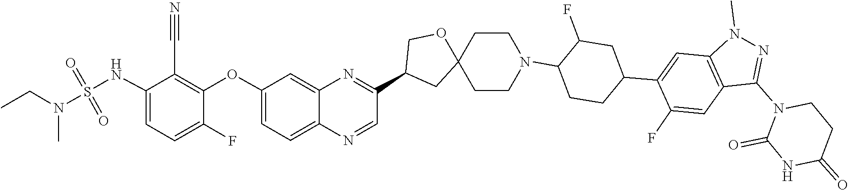 [SMILES: CCN(C)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6F)CC5)C4)nc3c2)c1C#N] Compound 160 Stereoisomer 2 | Compound 160 |
| 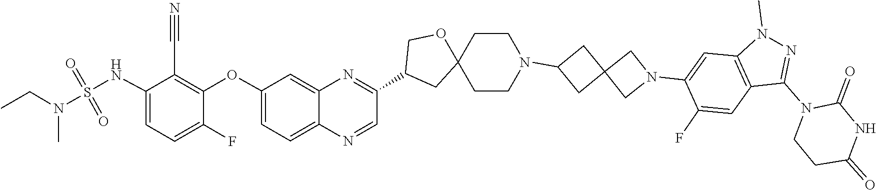 Compound 161 | Compound 161 |
| 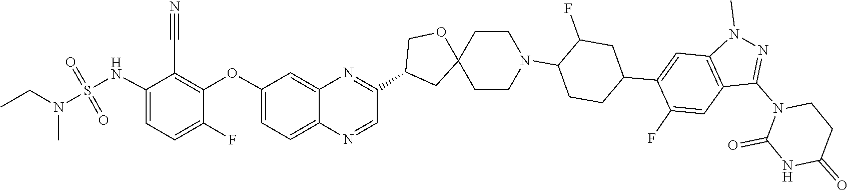 [SMILES: CCN(C)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6F)CC5)C4)nc3c2)c1C#N] Compound 162 Stereoisomer 1 | Compound 162 |
| 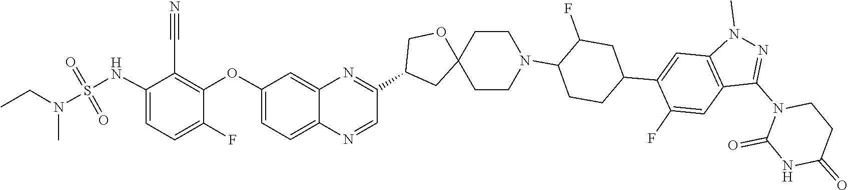 [SMILES: CCN(C)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6F)CC5)C4)nc3c2)c1C#N] Compound 163 Stereoisomer 2 | Compound 163 |
| 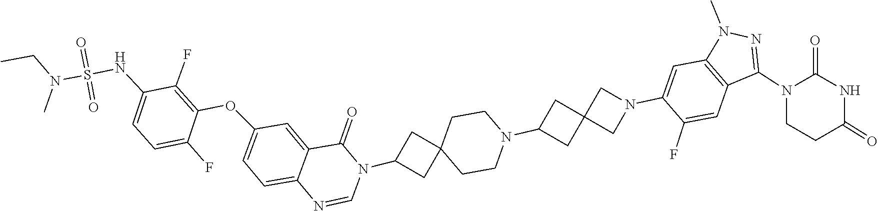 [SMILES: CCN(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1F] Compound 164 | Compound 164 |
| 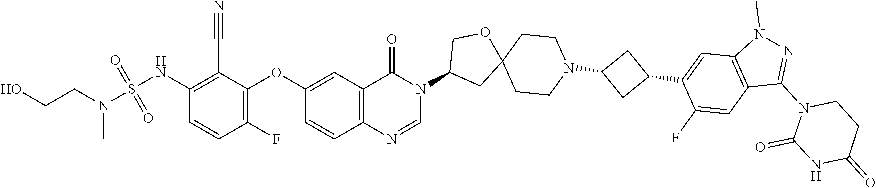 [SMILES: CN(CCO)S(=O)(=O)Nc1ccc(F)c(Oc2ccc3ncn([C@H]4COC5(CCN([C@H]6C[C@@H](c7cc8c(cc7F)c(N7CCC(=O)NC7=O)nn8C)C6)CC5)C4)c(=O)c3c2)c1C#N] Compound 165 | Compound 165 |
| 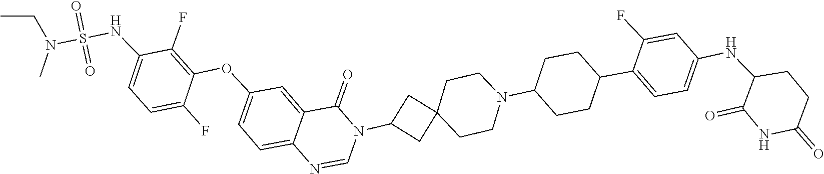 [SMILES: CCN(C)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1F] Compound 166 Stereoisomer 1 | Compound 166 |
| 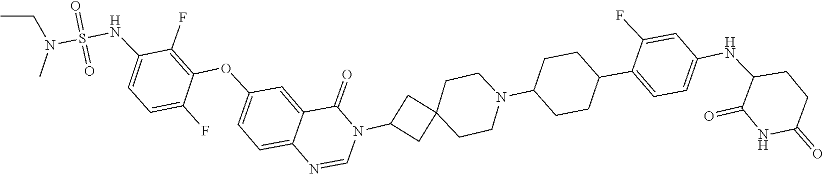 [SMILES: CCN(C)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1F] Compound 167 Stereoisomer 2 | Compound 167 |
| 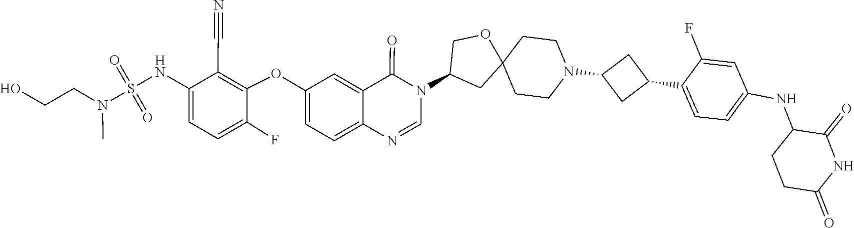 [SMILES: CN(CCO)S(=O)(=O)Nc1ccc(F)c(Oc2ccc3ncn([C@H]4COC5(CCN([C@H]6C[C@@H](c7ccc(NC8CCC(=O)NC8=O)cc7F)C6)CC5)C4)c(=O)c3c2)c1C#N] Compound 168 | Compound 168 |
| 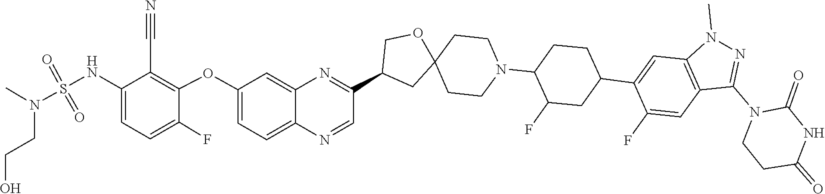 [SMILES: CN(CCO)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6F)CC5)C4)nc3c2)c1C#N] Compound 169 Stereoisomer 1 | Compound 169 |
| 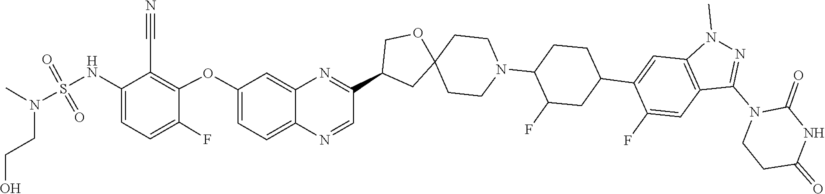 [SMILES: CN(CCO)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6F)CC5)C4)nc3c2)c1C#N] Compound 170 Stereoisomer 2 | Compound 170 |
| 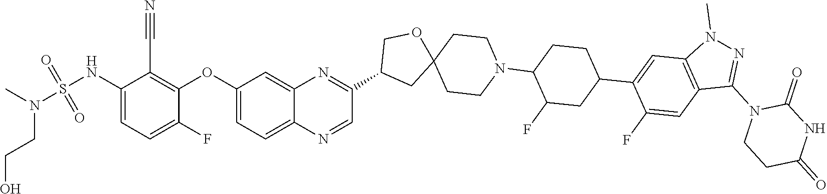 [SMILES: CN(CCO)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6F)CC5)C4)nc3c2)c1C#N] Compound 171 Stereoisomer 1 | Compound 171 |
| 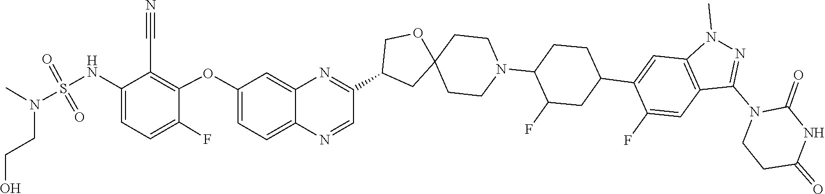 [SMILES: CN(CCO)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6F)CC5)C4)nc3c2)c1C#N] Compound 172 | Compound 172 |
| 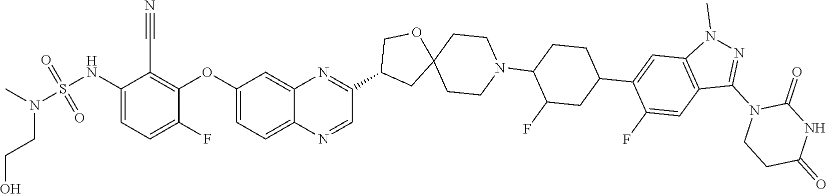 [SMILES: CN(CCO)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6F)CC5)C4)nc3c2)c1C#N] Compound 173 Stereoisomer 1 | Compound 173 |
| 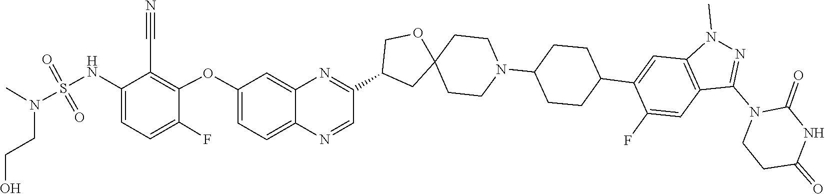 [SMILES: CN(CCO)S(=O)(=O)Nc1ccc(F)c(Oc2ccc3ncc([C@@H]4COC5(CCN(C6CCC(c7cc8c(cc7F)c(N7CCC(=O)NC7=O)nn8C)CC6)CC5)C4)nc3c2)c1C#N] Compound 174 Stereoisomer 2 | Compound 174 |
| 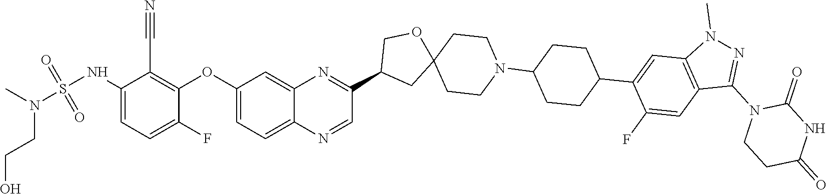 [SMILES: CN(CCO)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6)CC5)C4)nc3c2)c1C#N] Compound 175 Stereoisomer 1 | Compound 175 |
| 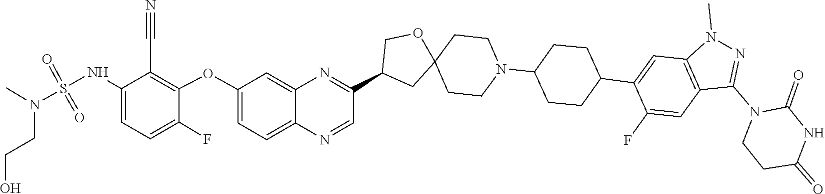 [SMILES: CN(CCO)S(=O)(=O)Nc1ccc(F)c(Oc2ccc3ncc([C@H]4COC5(CCN(C6CCC(c7cc8c(cc7F)c(N7CCC(=O)NC7=O)nn8C)CC6)CC5)C4)nc3c2)c1C#N] Compound 176 Stereoisomer 2 | Compound 176 |
| 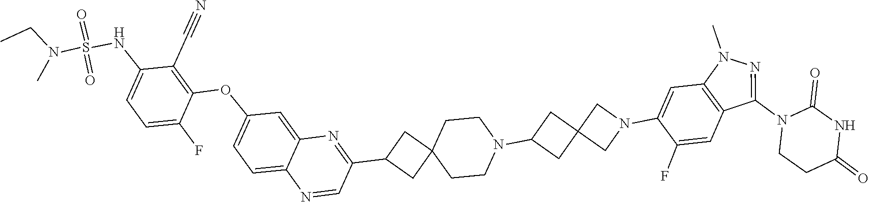 [SMILES: CCN(C)S(=O)(=O)Nc1ccc(F)c(Oc2ccc3ncc(C4CC5(CCN(C6CC7(C6)CN(c6cc8c(cc6F)c(N6CCC(=O)NC6=O)nn8C)C7)CC5)C4)nc3c2)c1C#N] Compound 177 | Compound 177 |
| 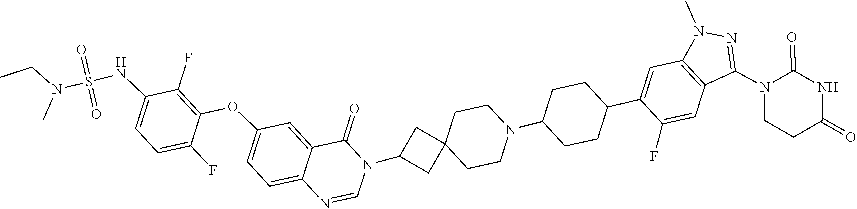 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6)CC5)C4)c(=O)c3c2)c1F] Compound 178 Stereoisomer 1 | Compound 178 |
| 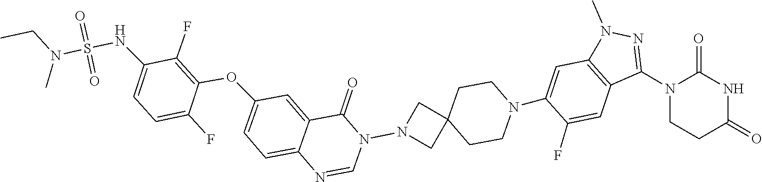 [SMILES: CCN(C)S(=O)(=O)Nc1ccc(F)c(Oc2ccc3ncn(N4CC5(CCN(c6cc7c(cc6F)c(N6CCC(=O)NC6=O)nn7C)CC5)C4)c(=O)c3c2)c1F] Compound 179 Stereoisomer 2 | Compound 179 |
| 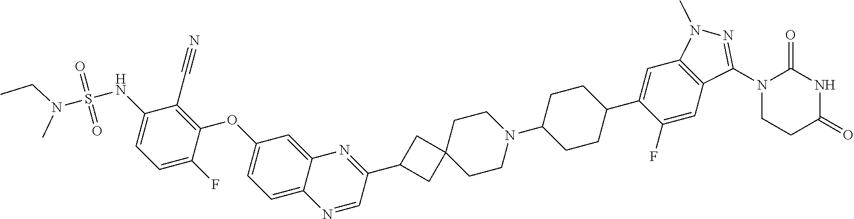 [SMILES: CCN(C)S(=O)(=O)Nc1ccc(F)c(Oc2ccc3ncc(C4CC5(CCN(C6CCC(c7cc8c(cc7F)c(N7CCC(=O)NC7=O)nn8C)CC6)CC5)C4)nc3c2)c1C#N] Compound 180 Stereoisomer 1 | Compound 180 |
| 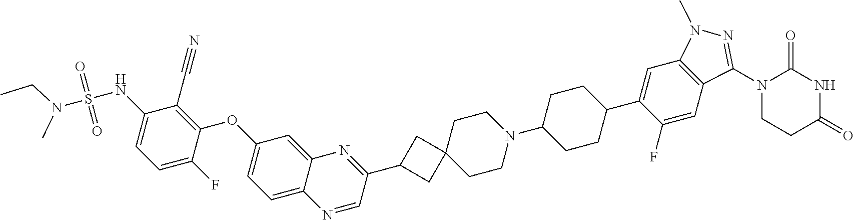 [SMILES: CCN(C)S(=O)(=O)Nc1ccc(F)c(Oc2ccc3ncc(C4CC5(CCN(C6CCC(c7cc8c(cc7F)c(N7CCC(=O)NC7=O)nn8C)CC6)CC5)C4)nc3c2)c1C#N] Compound 181 Stereoisomer 2 | Compound 181 |
| 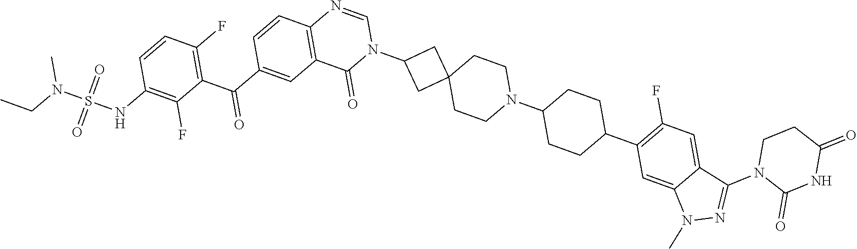 [SMILES: CCN(C)S(=O)(=O)Nc1ccc(F)c(C(=O)c2ccc3ncn(C4CC5(CCN(C6CCC(c7cc8c(cc7F)c(N7CCC(=O)NC7=O)nn8C)CC6)CC5)C4)c(=O)c3c2)c1F] Compound 182 Stereoisomer 1 | Compound 182 |
| 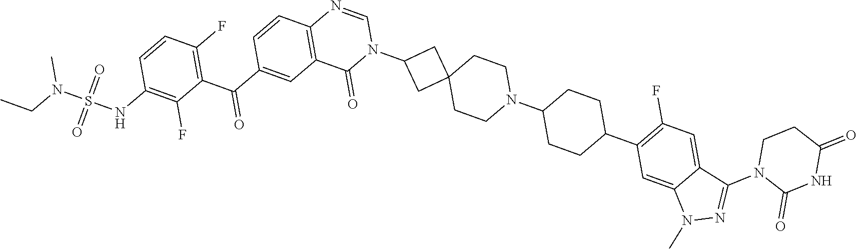 [SMILES: CCN(C)S(=O)(=O)Nc1ccc(F)c(C(=O)c2ccc3ncn(C4CC5(CCN(C6CCC(c7cc8c(cc7F)c(N7CCC(=O)NC7=O)nn8C)CC6)CC5)C4)c(=O)c3c2)c1F] Compound 183 Stereoisomer 2 | Compound 183 |
| 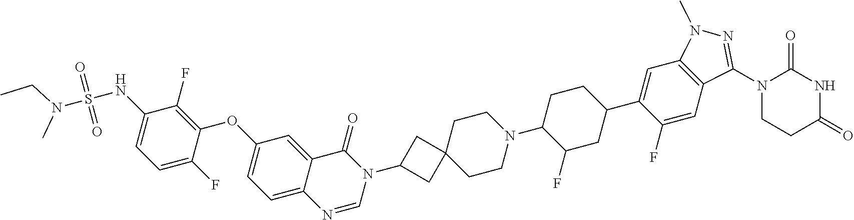 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1F] Compound 184 Stereoisomer 1 | Compound 184 |
| 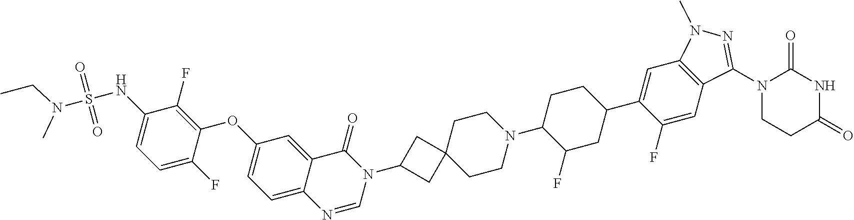 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6F)CC5)C4)c(=O)c3c2)c1F] Compound 185 Stereoisomer 2 | Compound 185 |
| 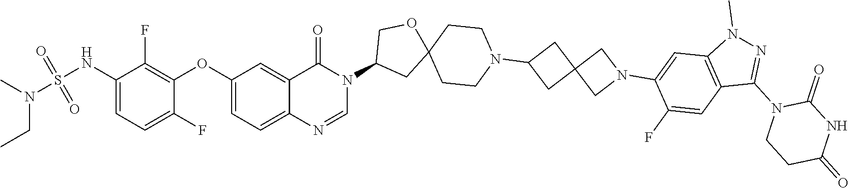 [SMILES: CCN(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1F] Compound 186 | Compound 186 |
| 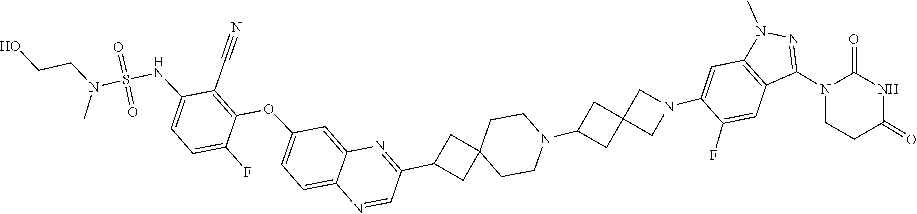 [SMILES: CN(CCO)S(=O)(=O)Nc1ccc(F)c(Oc2ccc3ncc(C4CC5(CCN(C6CC7(C6)CN(c6cc8c(cc6F)c(N6CCC(=O)NC6=O)nn8C)C7)CC5)C4)nc3c2)c1C#N] Compound 187 | Compound 187 |
| 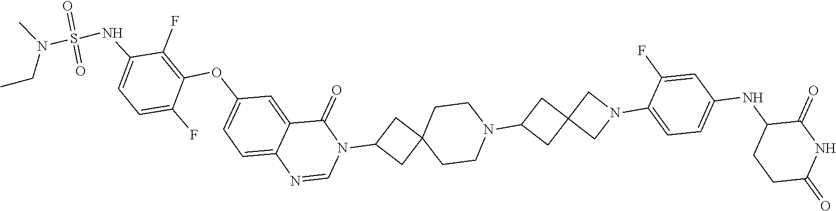 [SMILES: CCN(C)S(=O)(=O)Nc1ccc(F)c(Oc2ccc3ncn(C4CC5(CCN(C6CC7(C6)CN(c6ccc(NC8CCC(=O)NC8=O)cc6F)C7)CC5)C4)c(=O)c3c2)c1F] Compound 188 | Compound 188 |
| 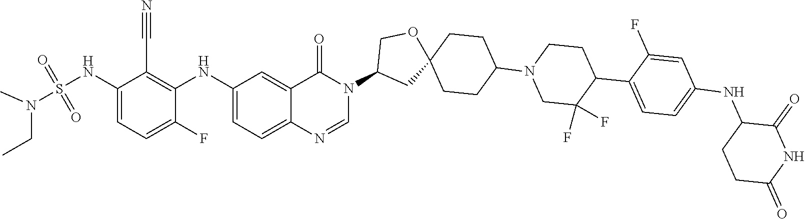 Compound 189 Stereoisomer 1 | Compound 189 |
| 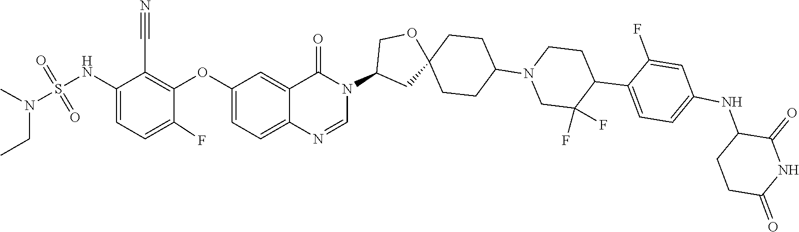 Compound 190 Stereoisomer 2 | Compound 190 |
| 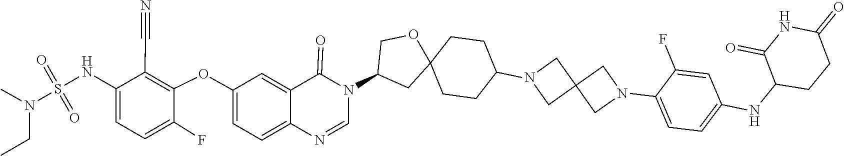 [SMILES: CCN(C)S(=O)(=O)Nc1ccc(F)c(Oc2ccc3ncn([C@H]4COC5(CCC(N6CC7(CN(c8ccc(NC9CCC(=O)NC9=O)cc8F)C7)C6)CC5)C4)c(=O)c3c2)c1C#N] Compound 191 Stereoisomer 1 | Compound 191 |
| 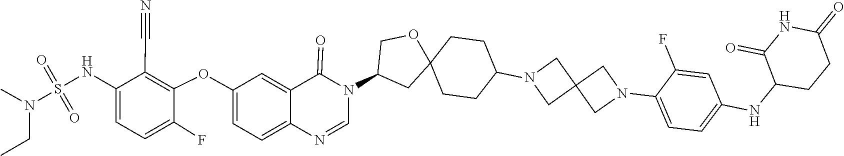 [SMILES: CCN(C)S(=O)(=O)Nc1ccc(F)c(Oc2ccc3ncn([C@H]4COC5(CCC(N6CC7(CN(c8ccc(NC9CCC(=O)NC9=O)cc8F)C7)C6)CC5)C4)c(=O)c3c2)c1C#N] Compound 192 Stereoisomer 2 | Compound 192 |
| 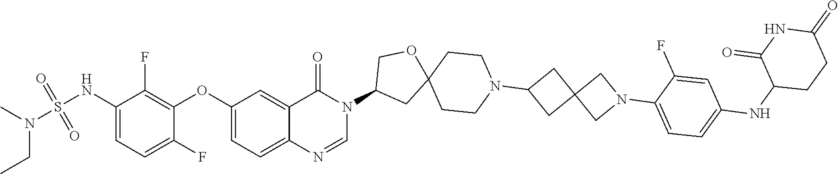 [SMILES: CCN(C)S(=O)(=O)Nc1ccc(F)c(Oc2ccc3ncn([C@H]4COC5(CCN(C6CC7(C6)CN(c6ccc(NC8CCC(=O)NC8=O)cc6F)C7)CC5)C4)c(=O)c3c2)c1F] Compound 193 | Compound 193 |
| 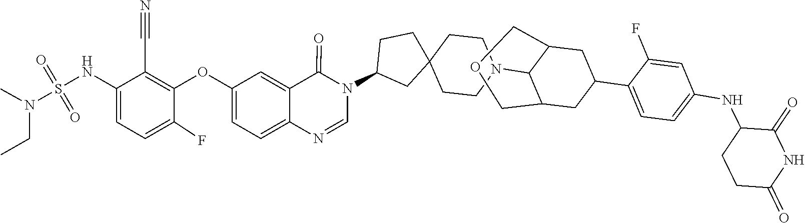 [SMILES: CCN(C)S(=O)(=O)Nc1ccc(F)c(Oc2ccc3ncn([C@H]4CCC5(CCN(C6C7COCC6CC(c6ccc(NC8CCC(=O)NC8=O)cc6F)C7)CC5)C4)c(=O)c3c2)c1C#N] Compound 194 | Compound 194 |
| 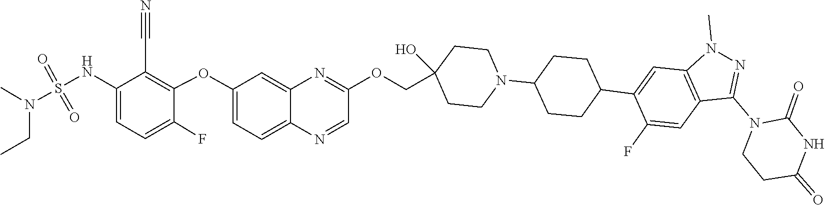 [SMILES: CCN(C)S(=O)(=O)Nc1ccc(F)c(Oc2ccc3ncc(OCC4(O)CCN(C5CCC(c6cc7c(cc6F)c(N6CCC(=O)NC6=O)nn7C)CC5)CC4)nc3c2)c1C#N] Compound 195 Stereoisomer 1 | Compound 195 |
| 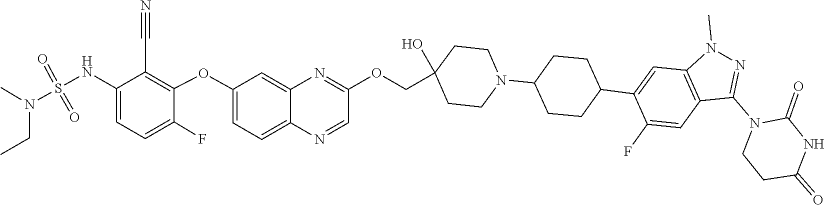 [SMILES: CCN(C)S(=O)(=O)Nc1ccc(F)c(Oc2ccc3ncc(OCC4(O)CCN(C5CCC(c6cc7c(cc6F)c(N6CCC(=O)NC6=O)nn7C)CC5)CC4)nc3c2)c1C#N] Compound 196 Stereoisomer 2 | Compound 196 |
| 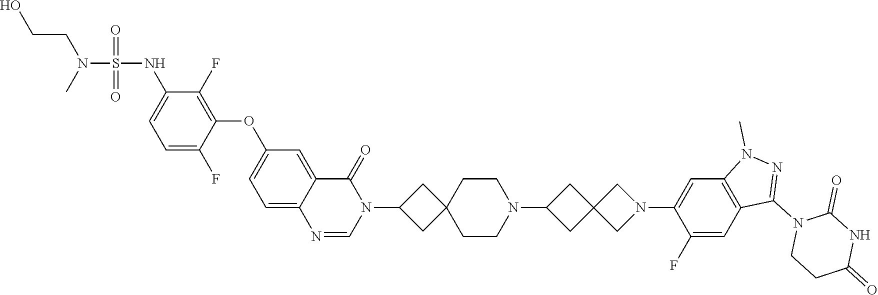 [SMILES: CN(CCO)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1F] Compound 197 | Compound 197 |
| 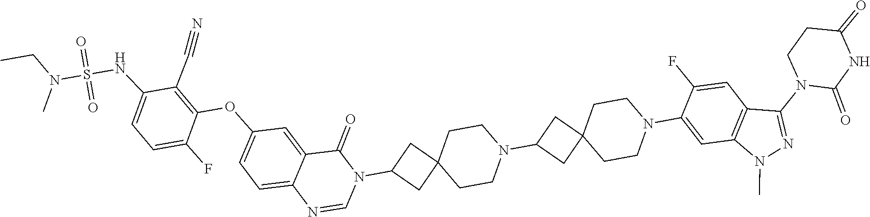 [SMILES: CCN(C)S(=O)(=O)Nc1ccc(F)c(Oc2ccc3ncn(C4CC5(CCN(C6CC7(CCN(c8cc9c(cc8F)c(N8CCC(=O)NC8=O)nn9C)CC7)C6)CC5)C4)c(=O)c3c2)c1C#N] Compound 198 | Compound 198 |
| 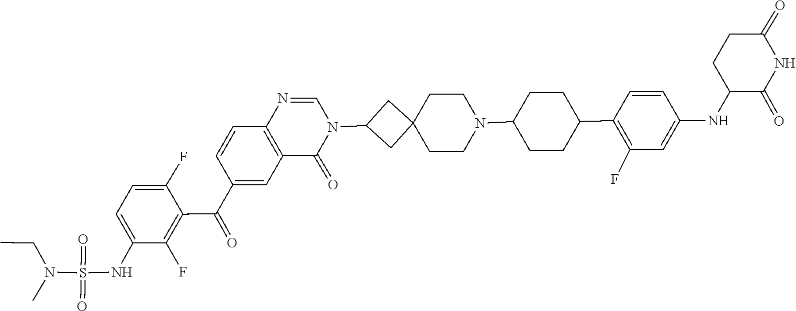 [SMILES: CCN(C)S(=O)(=O)Nc1ccc(F)c(C(=O)c2ccc3ncn(C4CC5(CCN(C6CCC(c7ccc(NC8CCC(=O)NC8=O)cc7F)CC6)CC5)C4)c(=O)c3c2)c1F] Compound 199 Stereoisomer 1 | Compound 199 |
| 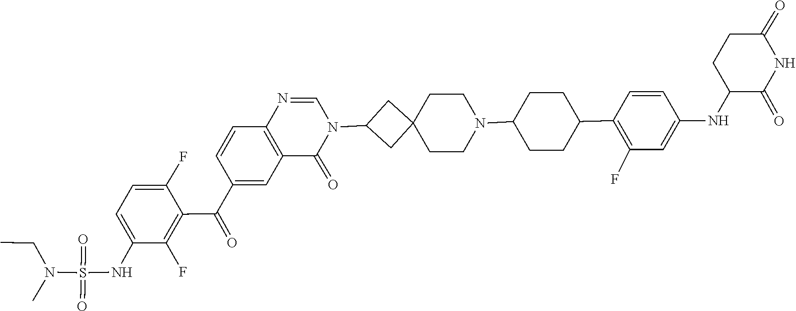 [SMILES: CCN(C)S(=O)(=O)Nc1ccc(F)c(C(=O)c2ccc3ncn(C4CC5(CCN(C6CCC(c7ccc(NC8CCC(=O)NC8=O)cc7F)CC6)CC5)C4)c(=O)c3c2)c1F] Compound 200 Stereoisomer 2 | Compound 200 |
| 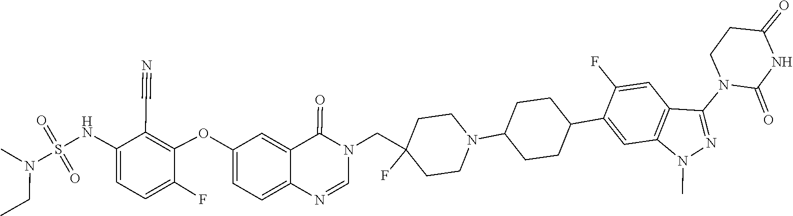 [SMILES: CCN(C)S(=O)(=O)Nc1ccc(F)c(Oc2ccc3ncn(CC4(F)CCN(C5CCC(c6cc7c(cc6F)c(N6CCC(=O)NC6=O)nn7C)CC5)CC4)c(=O)c3c2)c1C#N] Compound 201 Stereoisomer 1 | Compound 201 |
| 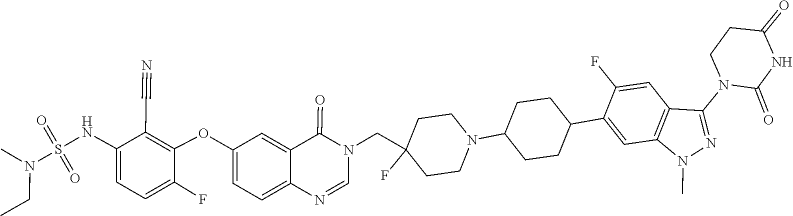 [SMILES: CCN(C)S(=O)(=O)Nc1ccc(F)c(Oc2ccc3ncn(CC4(F)CCN(C5CCC(c6cc7c(cc6F)c(N6CCC(=O)NC6=O)nn7C)CC5)CC4)c(=O)c3c2)c1C#N] Compound 202 Stereoisomer 2 | Compound 202 |
| 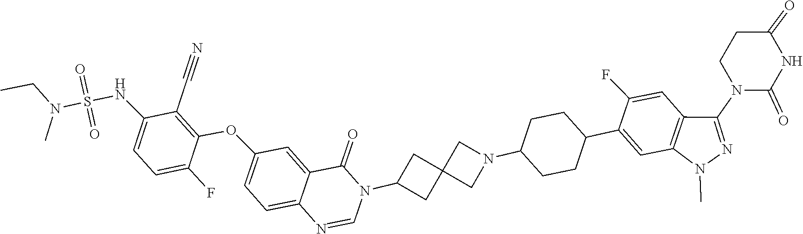 [SMILES: CCN(C)S(=O)(=O)Nc1ccc(F)c(Oc2ccc3ncn(C4CC5(C4)CN(C4CCC(c6cc7c(cc6F)c(N6CCC(=O)NC6=O)nn7C)CC4)C5)c(=O)c3c2)c1C#N] Compound 203 Stereoisomer 1 | Compound 2023 |
| 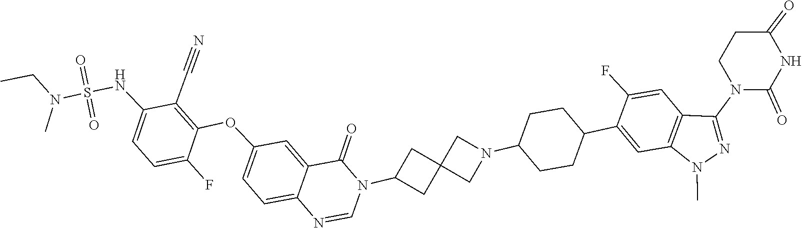 [SMILES: CCN(C)S(=O)(=O)Nc1ccc(F)c(Oc2ccc3ncn(C4CC5(C4)CN(C4CCC(c6cc7c(cc6F)c(N6CCC(=O)NC6=O)nn7C)CC4)C5)c(=O)c3c2)c1C#N] Compound 204 Stereoisomer 2 | Compound 204 |
| 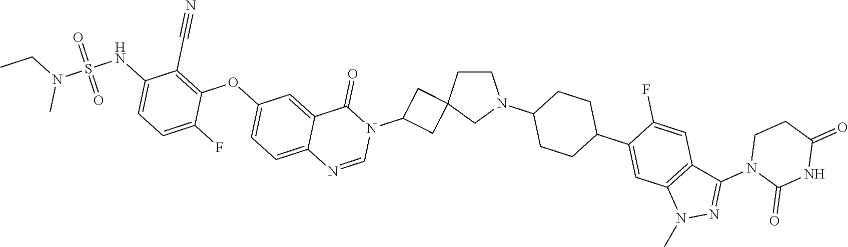 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6)C5)C4)c(=O)c3c2)c1C#N] Compound 205 Stereoisomer 1 | Compound 205 |
| 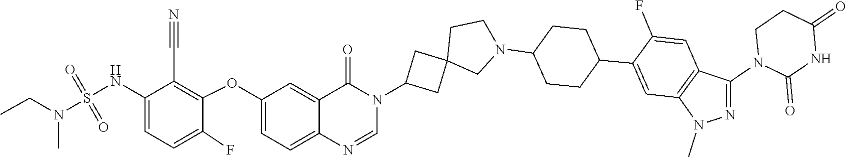 [SMILES: CCN(C)S(=O)(=O)Nc1ccc(F)c(Oc2ccc3ncn(C4CC5(CCN(C6CCC(c7cc8c(cc7F)c(N7CCC(=O)NC7=O)nn8C)CC6)C5)C4)c(=O)c3c2)c1C#N] Compound 206 Stereoisomer 2 | Compound 206 |
| 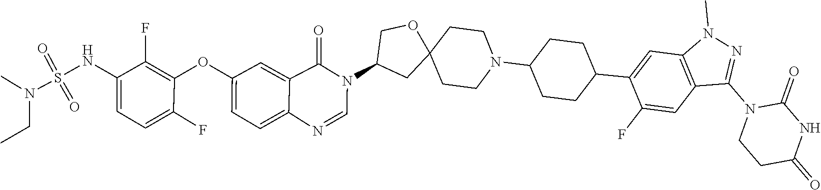 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1F] Compound 207 Stereoisomer 1 | Compound 207 |
| 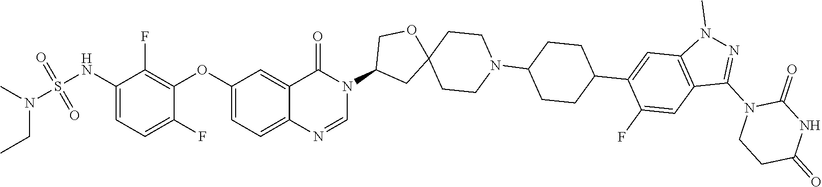 [SMILES: CCN(C)S(=O)(=O)Nc1ccc(F)c(Oc2ccc3ncn([C@H]4COC5(CCN(C6CCC(c7cc8c(cc7F)c(N7CCC(=O)NC7=O)nn8C)CC6)CC5)C4)c(=O)c3c2)c1F] Compound 208 Stereoisomer 2 | Compound 208 |
| 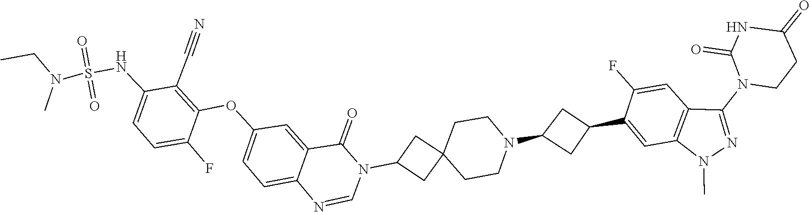 [SMILES: CCN(C)S(=O)(=O)Nc1ccc(F)c(Oc2ccc3ncn(C4CC5(CCN([C@H]6C[C@@H](c7cc8c(cc7F)c(N7CCC(=O)NC7=O)nn8C)C6)CC5)C4)c(=O)c3c2)c1C#N] Compound 209 | Compound 209 |
| 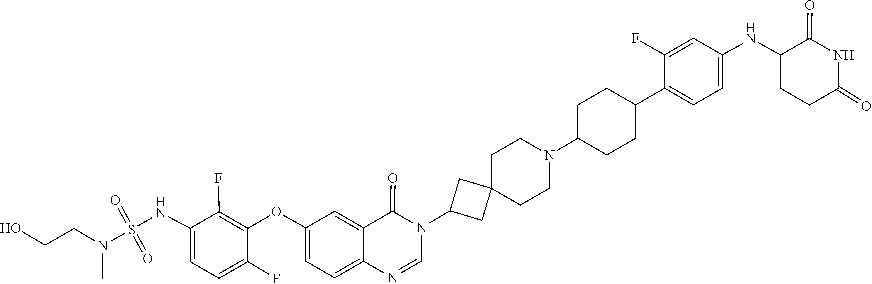 [SMILES: CN(CCO)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1F] Compound 210 Stereoisomer 1 | Compound 210 |
| 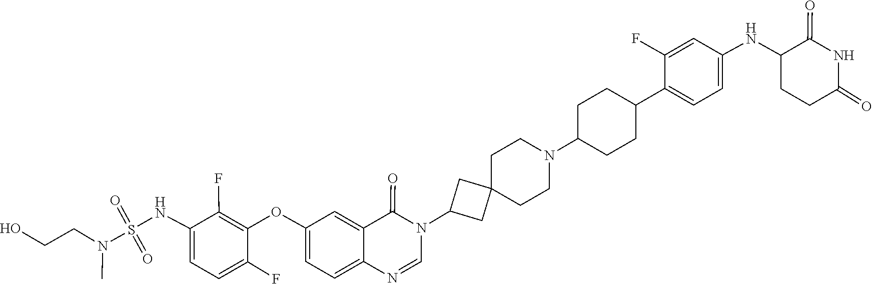 [SMILES: CN(CCO)S(=O)(=O)Nc1ccc(F)c(Oc2ccc3ncn(C4CC5(CCN(C6CCC(c7ccc(NC8CCC(=O)NC8=O)cc7F)CC6)CC5)C4)c(=O)c3c2)c1F] Compound 211 Stereoisomer 2 | Compound 211 |
| 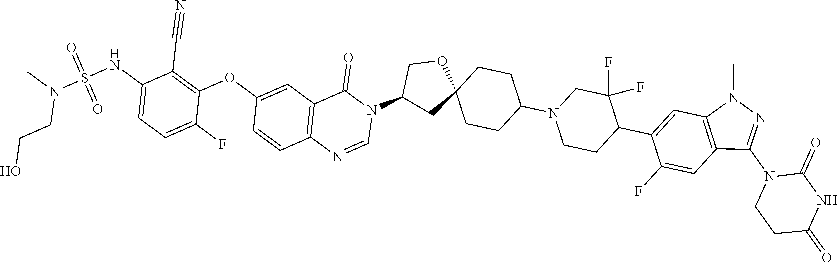 Compound 212 Stereoisomer 1 | Compound 212 |
| 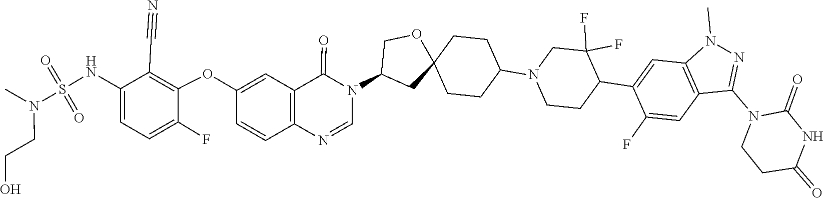 Compound 213 Stereoisomer 2 | Compound 213 |
| 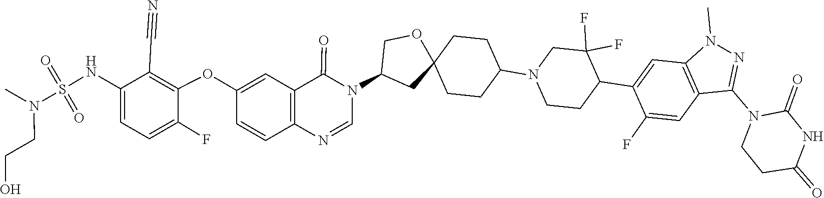 Compound 214 Stereoisomer 1 | Compound 214 |
| 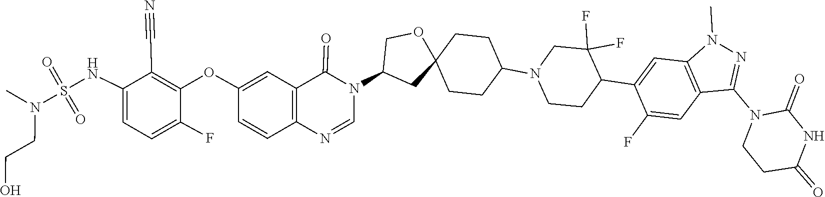 Compound 215 Stereoisomer 2 | Compound 215 |
| 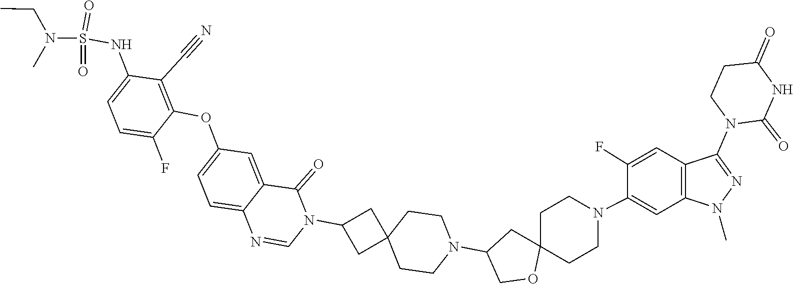 [SMILES: CCN(C)S(=O)(=O)Nc1ccc(F)c(Oc2ccc3ncn(C4CC5(CCN(C6COC7(CCN(c8cc9c(cc8F)c(N8CCC(=O)NC8=O)nn9C)CC7)C6)CC5)C4)c(=O)c3c2)c1C#N] Compound 216 | Compound 216 |
| 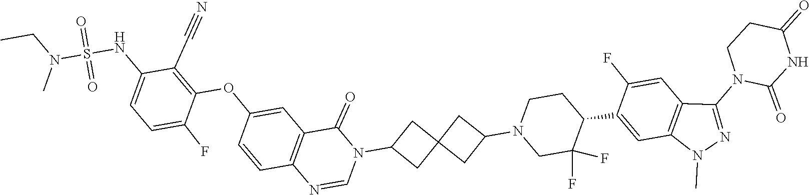 [SMILES: CCN(C)S(=O)(=O)Nc1ccc(F)c(Oc2ccc3ncn(C4CC5(CC(N6CC[C@H](c7cc8c(cc7F)c(N7CCC(=O)NC7=O)nn8C)C(F)(F)C6)C5)C4)c(=O)c3c2)c1C#N] Compound 217 | Compound 217 |
| 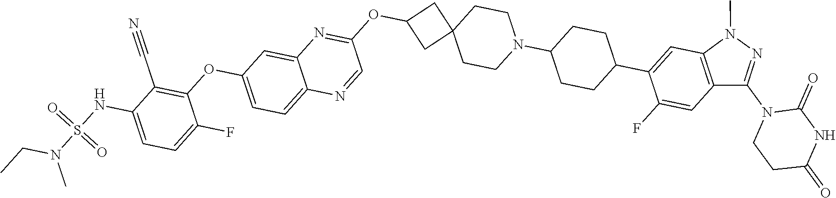 [SMILES: CCN(C)S(=O)(=O)Nc1ccc(F)c(Oc2ccc3ncc(OC4CC5(CCN(C6CCC(c7cc8c(cc7F)c(N7CCC(=O)NC7=O)nn8C)CC6)CC5)C4)nc3c2)c1C#N] Compound 218 Stereoisomer 1 | Compound 218 |
| 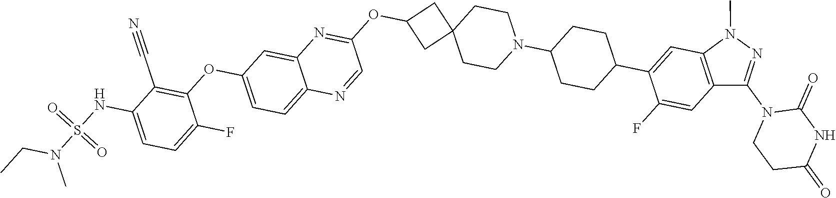 [SMILES: CCN(C)S(=O)(=O)Nc1ccc(F)c(Oc2ccc3ncc(OC4CC5(CCN(C6CCC(c7cc8c(cc7F)c(N7CCC(=O)NC7=O)nn8C)CC6)CC5)C4)nc3c2)c1C#N] Compound 219 Stereoisomer 2 | Compound 219 |
| 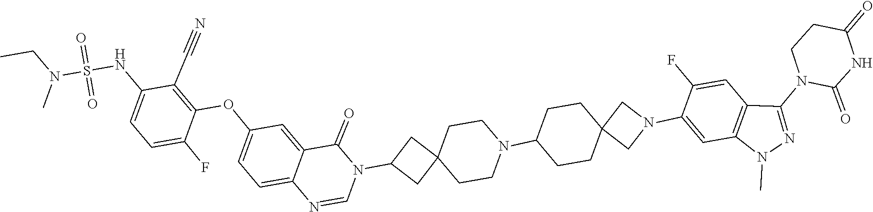 [SMILES: CCN(C)S(=O)(=O)Nc1ccc(F)c(Oc2ccc3ncn(C4CC5(CCN(C6CCC7(CC6)CN(c6cc8c(cc6F)c(N6CCC(=O)NC6=O)nn8C)C7)CC5)C4)c(=O)c3c2)c1C#N] Compound 220 | Compound 220 |
| 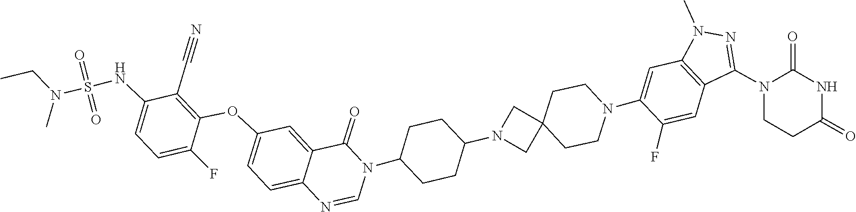 [SMILES: CCN(C)S(=O)(=O)Nc1ccc(F)c(Oc2ccc3ncn(C4CCC(N5CC6(CCN(c7cc8c(cc7F)c(N7CCC(=O)NC7=O)nn8C)CC6)C5)CC4)c(=O)c3c2)c1C#N] Compound 221 Stereoisomer 1 | Compound 221 |
| 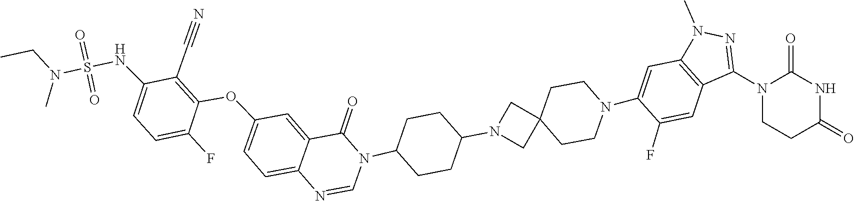 [SMILES: CCN(C)S(=O)(=O)Nc1ccc(F)c(Oc2ccc3ncn(C4CCC(N5CC6(CCN(c7cc8c(cc7F)c(N7CCC(=O)NC7=O)nn8C)CC6)C5)CC4)c(=O)c3c2)c1C#N] Compound 222 Stereoisomer 2 | Compound 222 |
| 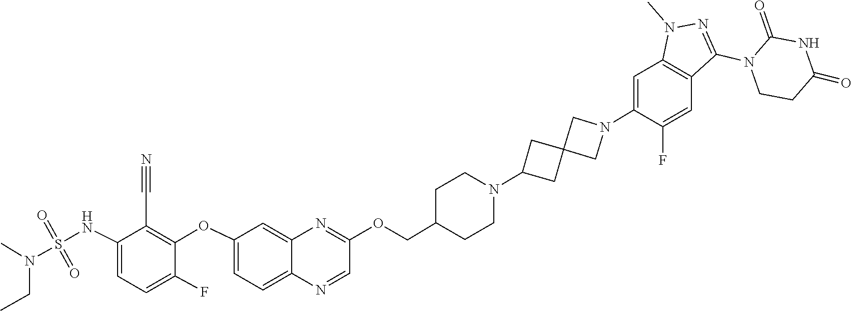 [SMILES: CCN(C)S(=O)(=O)Nc1ccc(F)c(Oc2ccc3ncc(OCC4CCN(C5CC6(C5)CN(c5cc7c(cc5F)c(N5CCC(=O)NC5=O)nn7C)C6)CC4)nc3c2)c1C#N] Compound 223 | Compound 223 |
| 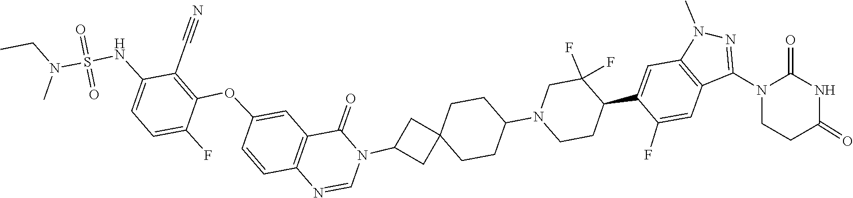 [SMILES: CCN(C)S(=O)(=O)Nc1ccc(F)c(Oc2ccc3ncn(C4CC5(CCC(N6CC[C@H](c7cc8c(cc7F)c(N7CCC(=O)NC7=O)nn8C)C(F)(F)C6)CC5)C4)c(=O)c3c2)c1C#N] Compound 224 | Compound 224 |
| 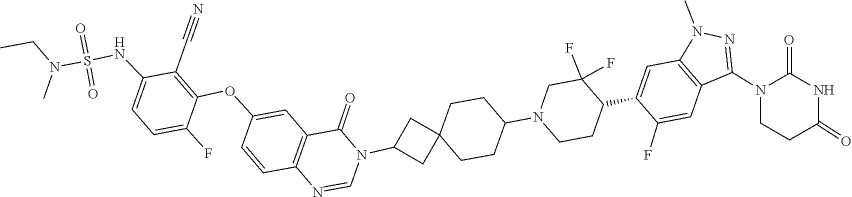 Compound 225 | Compound 225 |
| 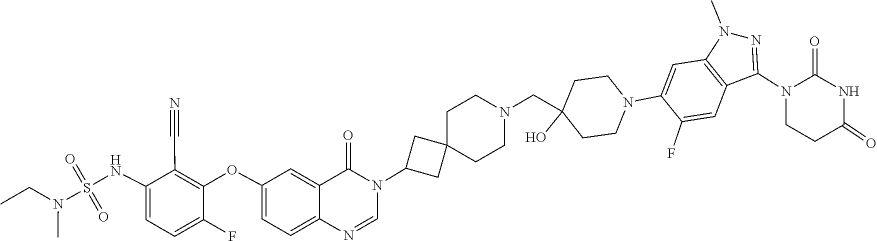 [SMILES: CCN(C)S(=O)(=O)Nc1ccc(F)c(Oc2ccc3ncn(C4CC5(CCN(CC6(O)CCN(c7cc8c(cc7F)c(N7CCC(=O)NC7=O)nn8C)CC6)CC5)C4)c(=O)c3c2)c1C#N] Compound 226 | Compound 226 |
| 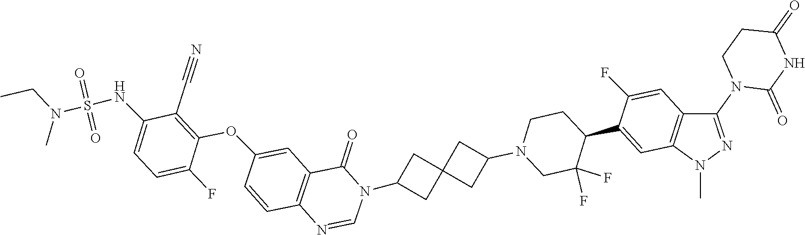 [SMILES: CCN(C)S(=O)(=O)Nc1ccc(F)c(Oc2ccc3ncn(C4CC5(CC(N6CC[C@@H](c7cc8c(cc7F)c(N7CCC(=O)NC7=O)nn8C)C(F)(F)C6)C5)C4)c(=O)c3c2)c1C#N] Compound 227 | Compound 227 |
| 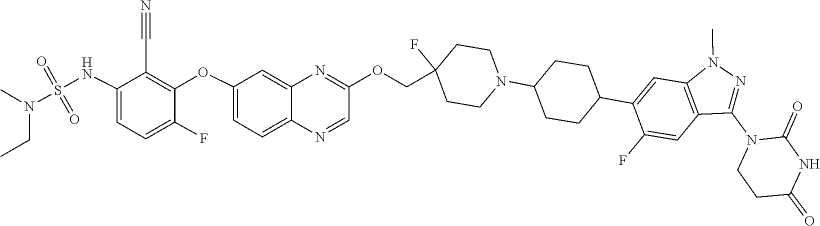 [SMILES: CCN(C)S(=O)(=O)Nc1ccc(F)c(Oc2ccc3ncc(OCC4(F)CCN(C5CCC(c6cc7c(cc6F)c(N6CCC(=O)NC6=O)nn7C)CC5)CC4)nc3c2)c1C#N] Compound 228 Stereoisomer 1 | Compound 228 |
| 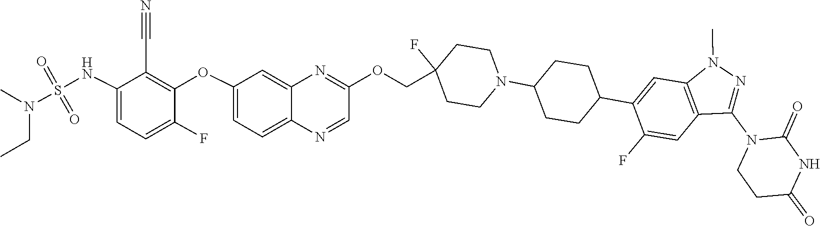 [SMILES: CCN(C)S(=O)(=O)Nc1ccc(F)c(Oc2ccc3ncc(OCC4(F)CCN(C5CCC(c6cc7c(cc6F)c(N6CCC(=O)NC6=O)nn7C)CC5)CC4)nc3c2)c1C#N] Compound 229 Stereoisomer 2 | Compound 229 |
| 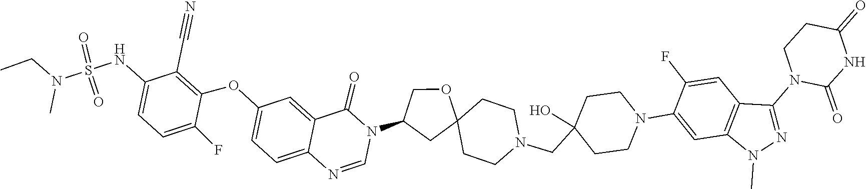 [SMILES: CCN(C)S(=O)(=O)Nc1ccc(F)c(Oc2ccc3ncn([C@H]4COC5(CCN(CC6(O)CCN(c7cc8c(cc7F)c(N7CCC(=O)NC7=O)nn8C)CC6)CC5)C4)c(=O)c3c2)c1C#N] Compound 230 | Compound 230 |
| 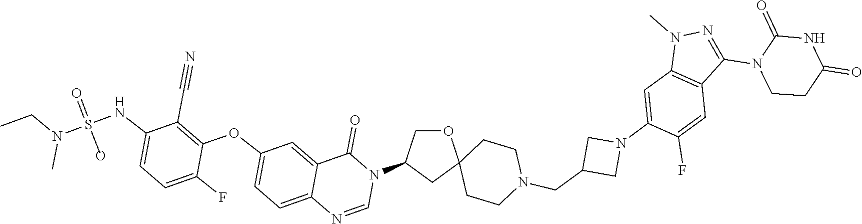 Compound 231 | Compound 231 |
| 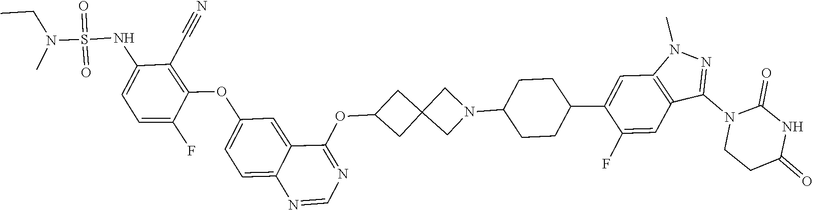 [SMILES: C=C1/C=C(F)\C(C2CCC(N3CC4(CC(Oc5ncnc6ccc(Oc7c(F)ccc(NS(=O)(=O)N(C)CC)c7C#N)cc56)C4)C3)CC2)=C/CN(C)/N=C\1N1CCC(=O)NC1=O] Compound 232 Stereoisomer 1 | Compound 232 |
| 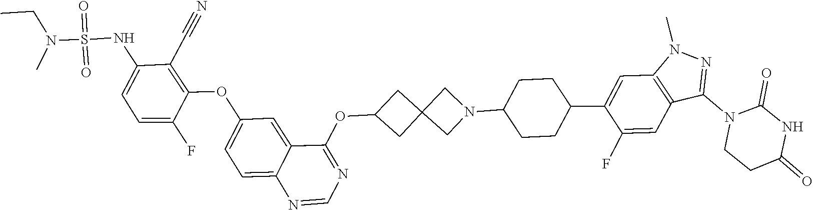 [SMILES: C=C1/C=C(F)\C(C2CCC(N3CC4(CC(Oc5ncnc6ccc(Oc7c(F)ccc(NS(=O)(=O)N(C)CC)c7C#N)cc56)C4)C3)CC2)=C/CN(C)/N=C\1N1CCC(=O)NC1=O] Compound 233 Stereoisomer 2 | Compound 233 |
| 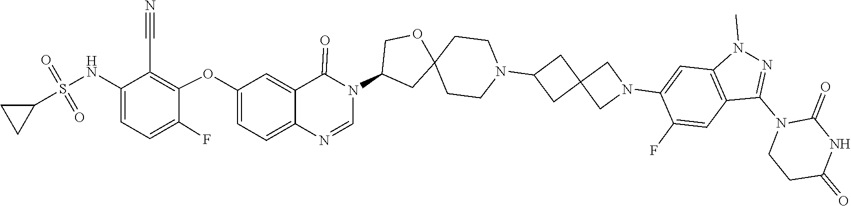 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CC9)c8C#N)cc7c5=O)CO6)C4)C3)cc21] Compound 234 | Compound 234 |
| 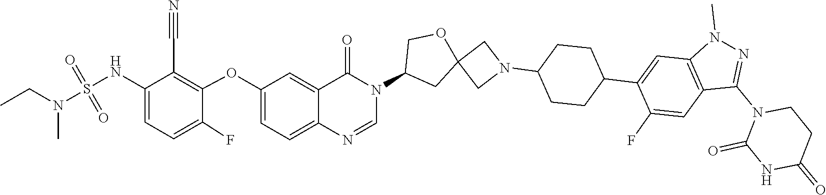 [SMILES: CCN(C)S(=O)(=O)Nc1ccc(F)c(Oc2ccc3ncn([C@H]4COC5(C4)CN(C4CCC(c6cc7c(cc6F)c(N6CCC(=O)NC6=O)nn7C)CC4)C5)c(=O)c3c2)c1C#N] Compound 235 Stereoisomer 1 | Compound 235 |
| 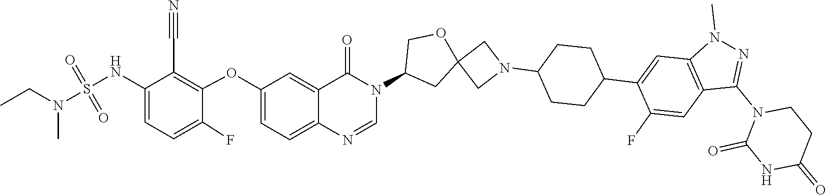 [SMILES: CCN(C)S(=O)(=O)Nc1ccc(F)c(Oc2ccc3ncn([C@H]4COC5(C4)CN(C4CCC(c6cc7c(cc6F)c(N6CCC(=O)NC6=O)nn7C)CC4)C5)c(=O)c3c2)c1C#N] Compound 236 Stereoisomer 2 | Compound 236 |
| 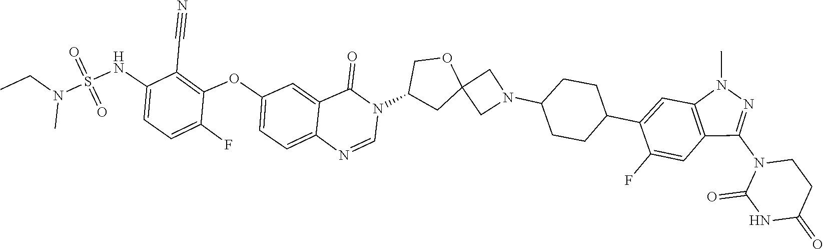 [SMILES: CCN(C)S(=O)(=O)Nc1ccc(F)c(Oc2ccc3ncn([C@@H]4COC5(C4)CN(C4CCC(c6cc7c(cc6F)c(N6CCC(=O)NC6=O)nn7C)CC4)C5)c(=O)c3c2)c1C#N] Compound 237 Stereoisomer 1 | Compound 237 |
| 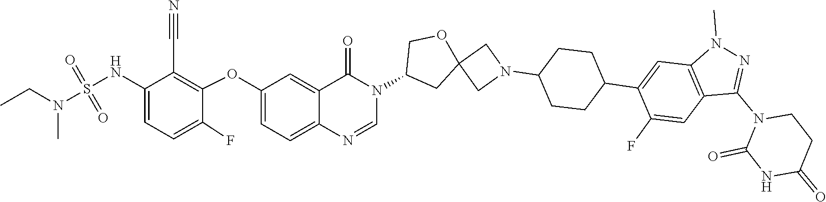 [SMILES: CCN(C)S(=O)(=O)Nc1ccc(F)c(Oc2ccc3ncn([C@@H]4COC5(C4)CN(C4CCC(c6cc7c(cc6F)c(N6CCC(=O)NC6=O)nn7C)CC4)C5)c(=O)c3c2)c1C#N] Compound 238 Stereoisomer 2 | Compound 238 |
| 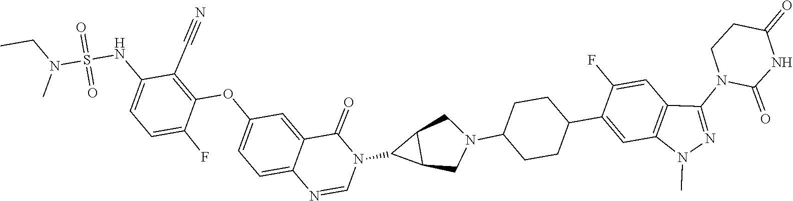 Compound 239 Stereoisomer 1 | Compound 239 |
| 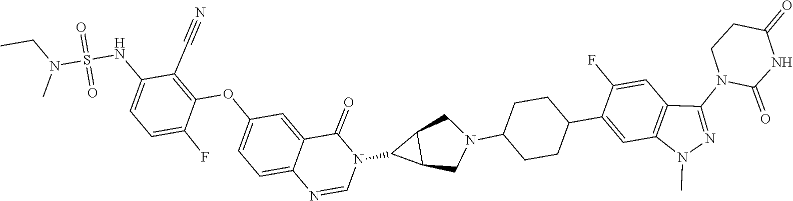 Compound 240 Stereoisomer 2 | Compound 240 |
| 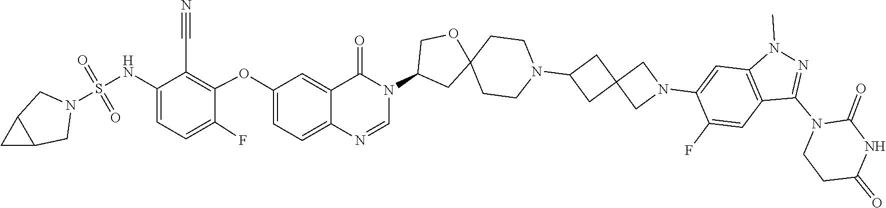 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC%10CC%10C9)c8C#N)cc7c5=O)CO6)C4)C3)cc21] Compound 241 | Compound 241 |
| 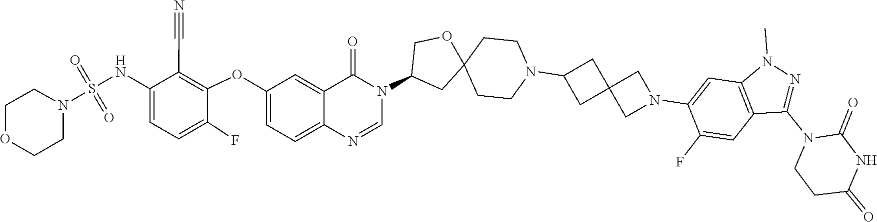 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CCOCC9)c8C#N)cc7c5=O)CO6)C4)C3)cc21] Compound 242 | Compound 242 |
| 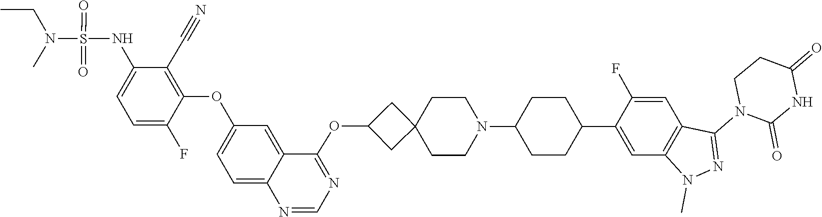 [SMILES: CCN(C)S(=O)(=O)Nc1ccc(F)c(Oc2ccc3ncnc(OC4CC5(CCN(C6CCC(c7cc8c(cc7F)c(N7CCC(=O)NC7=O)nn8C)CC6)CC5)C4)c3c2)c1C#N] Compound 243 Stereoisomer 1 | Compound 243 |
| 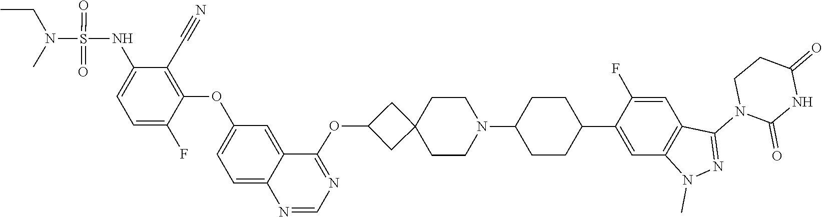 [SMILES: CCN(C)S(=O)(=O)Nc1ccc(F)c(Oc2ccc3ncnc(OC4CC5(CCN(C6CCC(c7cc8c(cc7F)c(N7CCC(=O)NC7=O)nn8C)CC6)CC5)C4)c3c2)c1C#N] Compound 244 Stereoisomer 2 | Compound 244 |
| 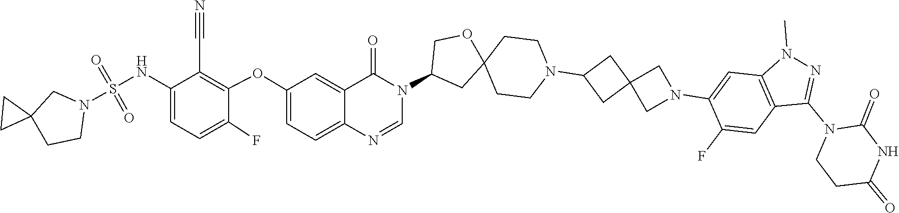 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CCC%10(CC%10)C9)c8C#N)cc7c5=O)CO6)C4)C3)cc21] Compound 245 | Compound 245 |
| 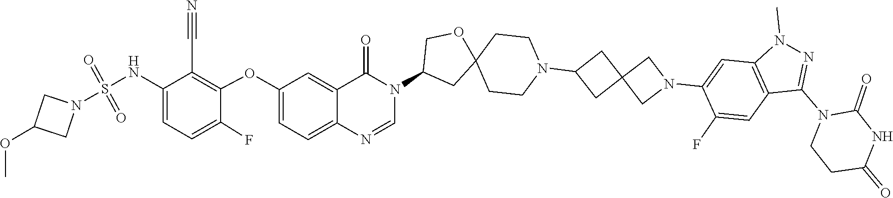 [SMILES: COC1CN(S(=O)(=O)Nc2ccc(F)c(Oc3ccc4ncn([C@H]5COC6(CCN(C7CC8(C7)CN(c7cc9c(cc7F)c(N7CCC(=O)NC7=O)nn9C)C8)CC6)C5)c(=O)c4c3)c2C#N)C1] Compound 246 | Compound 246 |
| 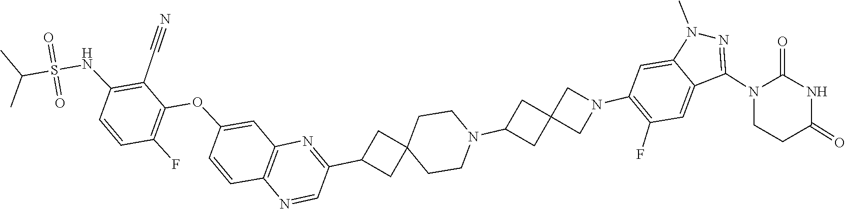 [SMILES: CC(C)S(=O)(=O)Nc1ccc(F)c(Oc2ccc3ncc(C4CC5(CCN(C6CC7(C6)CN(c6cc8c(cc6F)c(N6CCC(=O)NC6=O)nn8C)C7)CC5)C4)nc3c2)c1C#N] Compound 247 | Compound 247 |
| 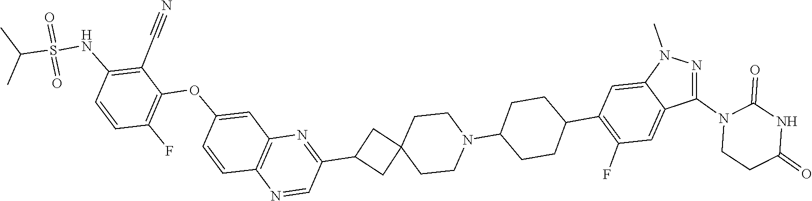 [SMILES: CC(C)S(=O)(=O)Nc1ccc(F)c(Oc2ccc3ncc(C4CC5(CCN(C6CCC(c7cc8c(cc7F)c(N7CCC(=O)NC7=O)nn8C)CC6)CC5)C4)nc3c2)c1C#N] Compound 248 Stereoisomer 1 | Compound 248 |
| 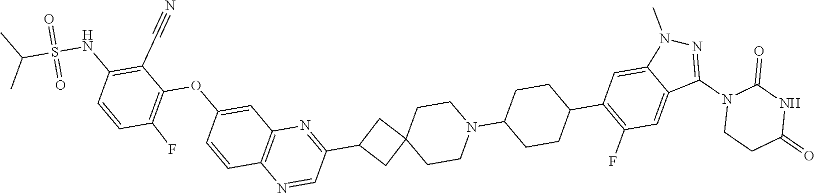 [SMILES: CC(C)S(=O)(=O)Nc1ccc(F)c(Oc2ccc3ncc(C4CC5(CCN(C6CCC(c7cc8c(cc7F)c(N7CCC(=O)NC7=O)nn8C)CC6)CC5)C4)nc3c2)c1C#N] Compound 249 Stereoisomer 2 | Compound 249 |
| 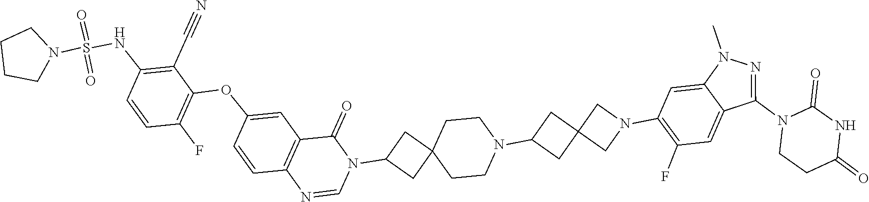 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9CCCC9)c8C#N)cc7c5=O)C6)C4)C3)cc21] Compound 250 | Compound 250 |
| 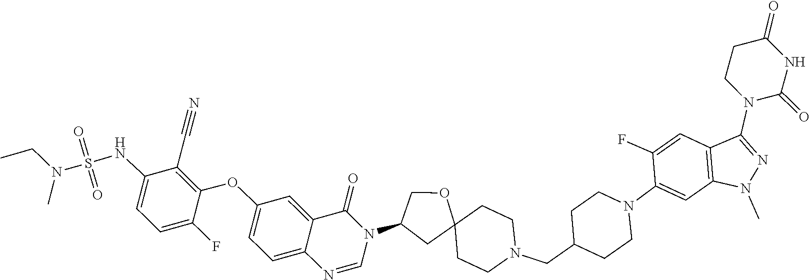 [SMILES: CCN(C)S(=O)(=O)Nc1ccc(F)c(Oc2ccc3ncn([C@H]4COC5(CCN(CC6CCN(c7cc8c(cc7F)c(N7CCC(=O)NC7=O)nn8C)CC6)CC5)C4)c(=O)c3c2)c1C#N] Compound 251 | Compound 251 |
| 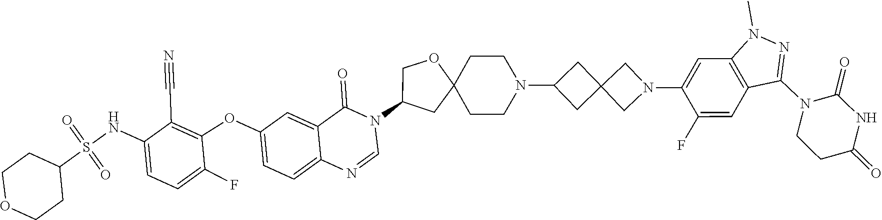 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CCOCC9)c8C#N)cc7c5=O)CO6)C4)C3)cc21] Compound 252 | Compound 252 |
| 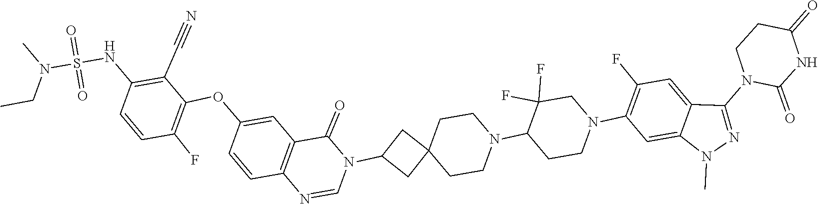 [SMILES: CCN(C)S(=O)(=O)Nc1ccc(F)c(Oc2ccc3ncn(C4CC5(CCN(C6CCN(c7cc8c(cc7F)c(N7CCC(=O)NC7=O)nn8C)CC6(F)F)CC5)C4)c(=O)c3c2)c1C#N] Compound 253 | Compound 253 |
| 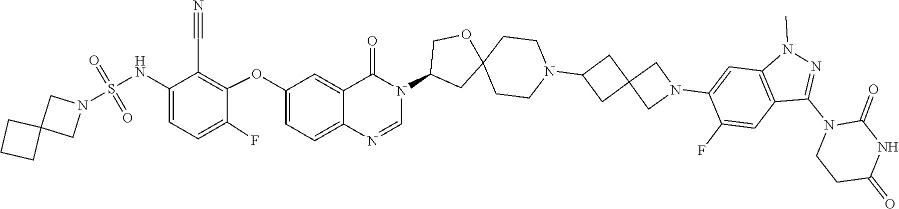 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC%10(CCC%10)C9)c8C#N)cc7c5=O)CO6)C4)C3)cc21] Compound 254 | Compound 254 |
| 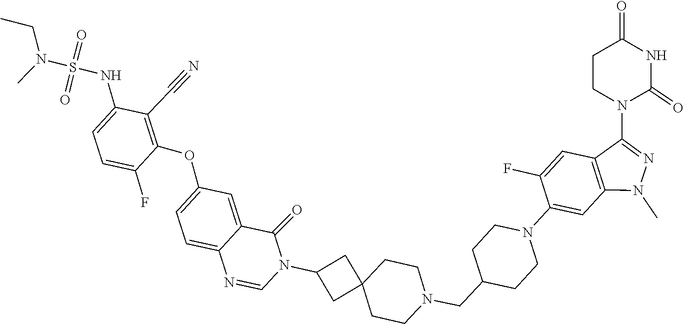 [SMILES: CCN(C)S(=O)(=O)Nc1ccc(F)c(Oc2ccc3ncn(C4CC5(CCN(CC6CCN(c7cc8c(cc7F)c(N7CCC(=O)NC7=O)nn8C)CC6)CC5)C4)c(=O)c3c2)c1C#N] Compound 255 | Compound 255 |
| 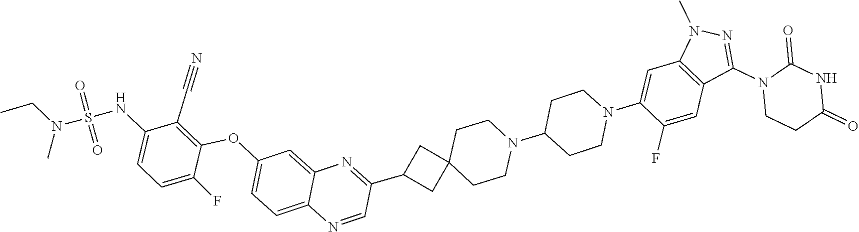 [SMILES: CCN(C)S(=O)(=O)Nc1ccc(F)c(Oc2ccc3ncc(C4CC5(CCN(C6CCN(c7cc8c(cc7F)c(N7CCC(=O)NC7=O)nn8C)CC6)CC5)C4)nc3c2)c1C#N] Compound 256 | Compound 256 |
| 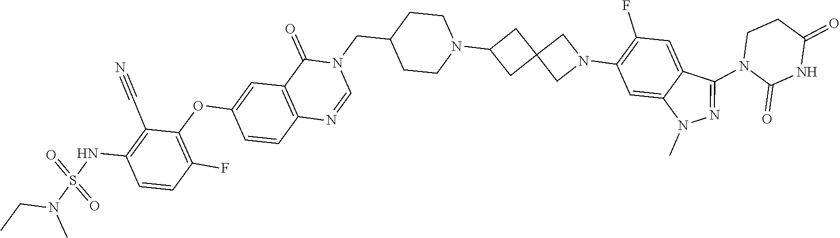 [SMILES: CCN(C)S(=O)(=O)Nc1ccc(F)c(Oc2ccc3ncn(CC4CCN(C5CC6(C5)CN(c5cc7c(cc5F)c(N5CCC(=O)NC5=O)nn7C)C6)CC4)c(=O)c3c2)c1C#N] Compound 258 | Compound 258 |
| 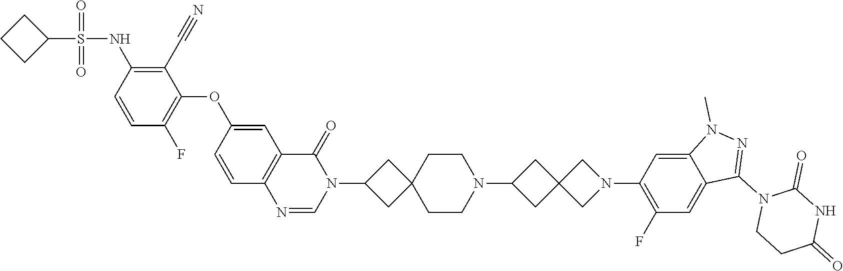 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)C9CCC9)c8C#N)cc7c5=O)C6)C4)C3)cc21] Compound 259 | Compound 259 |
| 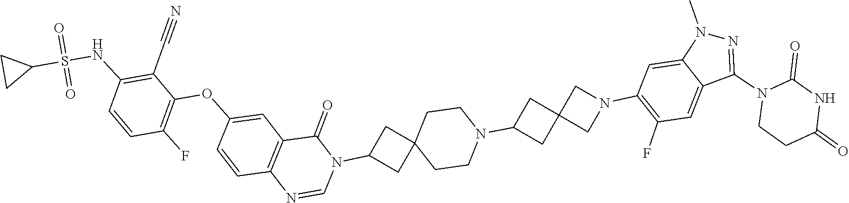 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)C9CC9)c8C#N)cc7c5=O)C6)C4)C3)cc21] Compound 260 | Compound 260 |
| 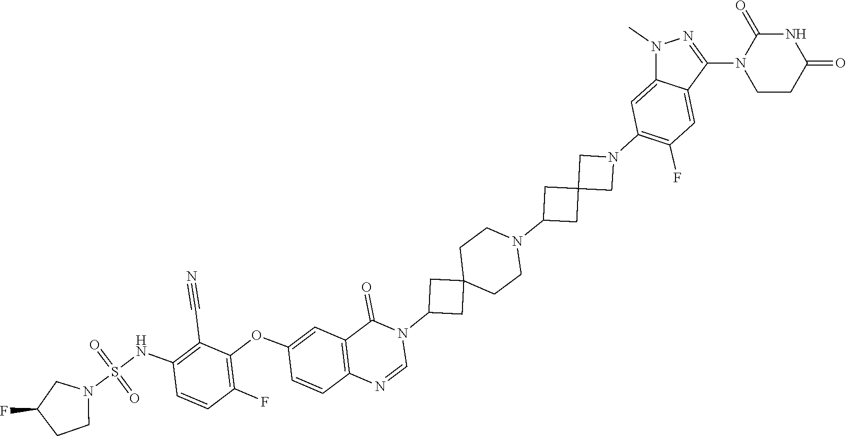 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9CC[C@@H](F)C9)c8C#N)cc7c5=O)C6)C4)C3)cc21] Compound 261 | Compound 261 |
| 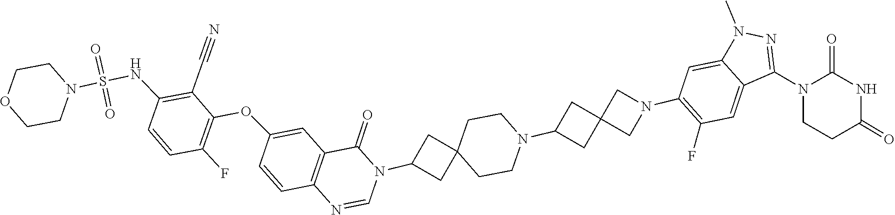 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9CCOCC9)c8C#N)cc7c5=O)C6)C4)C3)cc21] Compound 262 | Compound 262 |
| 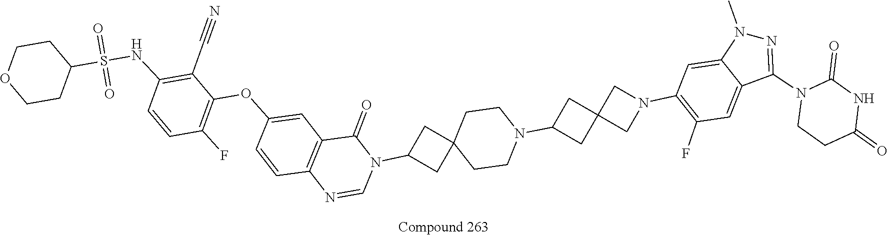 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)C9CCOCC9)c8C#N)cc7c5=O)C6)C4)C3)cc21] Compound 263 | Compound 263 |
| 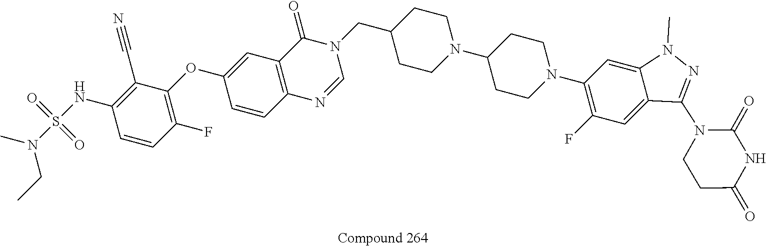 [SMILES: CCN(C)S(=O)(=O)Nc1ccc(F)c(Oc2ccc3ncn(CC4CCN(C5CCN(c6cc7c(cc6F)c(N6CCC(=O)NC6=O)nn7C)CC5)CC4)c(=O)c3c2)c1C#N] Compound 264 | Compound 264 |
| 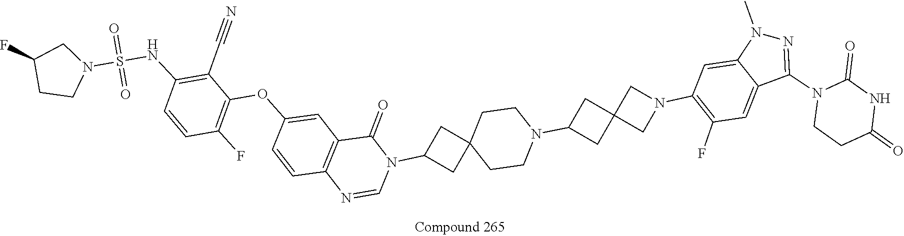 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9CC[C@@H](F)C9)c8C#N)cc7c5=O)C6)C4)C3)cc21] Compound 265 | Compound 265 |
| 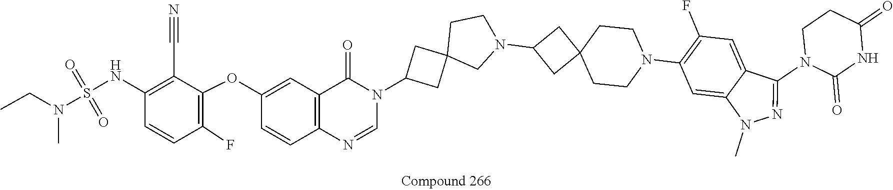 [SMILES: CCN(C)S(=O)(=O)Nc1ccc(F)c(Oc2ccc3ncn(C4CC5(CCN(C6CC7(CCN(c8cc9c(cc8F)c(N8CCC(=O)NC8=O)nn9C)CC7)C6)C5)C4)c(=O)c3c2)c1C#N] Compound 266 | Compound 266 |
| 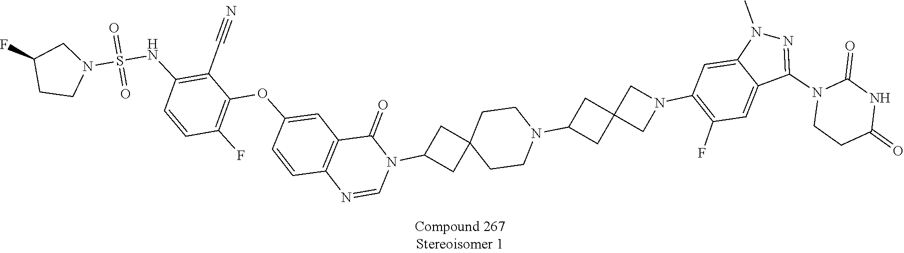 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9CC[C@@H](F)C9)c8C#N)cc7c5=O)C6)C4)C3)cc21] Compound 267 Stereoisomer 1 | Compound 267 |
| 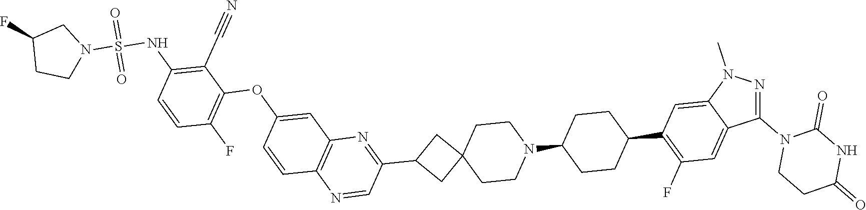 [SMILES: Cn1nc(N2CCC(=O)NC2=O)c2cc(F)c([C@H]3CC[C@@H](N4CCC5(CC4)CC(c4cnc6ccc(Oc7c(F)ccc(NS(=O)(=O)N8CC[C@@H](F)C8)c7C#N)cc6n4)C5)CC3)cc21] | Compound 268 |
| 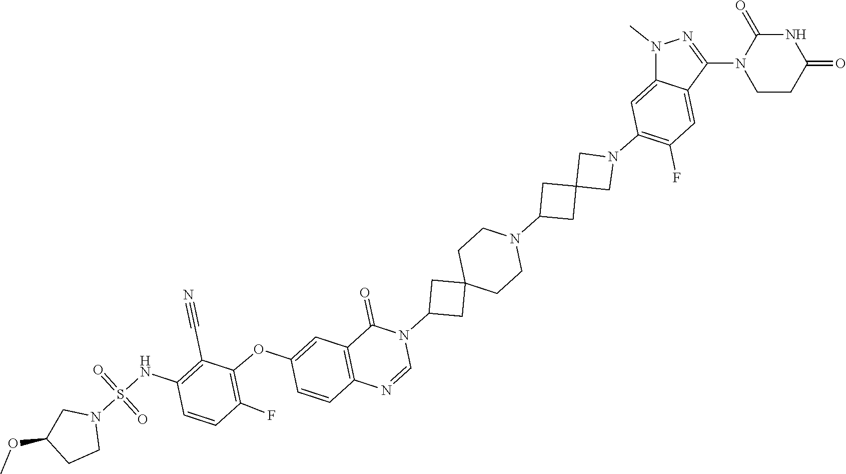 [SMILES: CO[C@@H]1CCN(S(=O)(=O)Nc2ccc(F)c(Oc3ccc4ncn(C5CC6(CCN(C7CC8(C7)CN(c7cc9c(cc7F)c(N7CCC(=O)NC7=O)nn9C)C8)CC6)C5)c(=O)c4c3)c2C#N)C1] Compound 269 | Compound 269 |
| 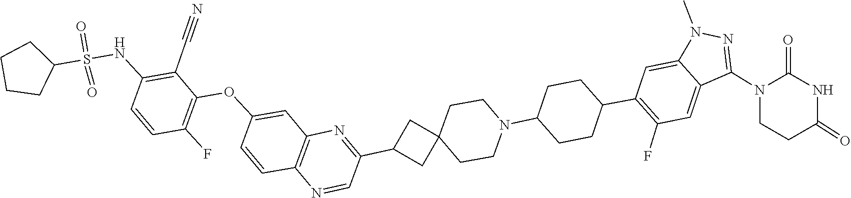 [SMILES: Cn1nc(N2CCC(=O)NC2=O)c2cc(F)c(C3CCC(N4CCC5(CC4)CC(c4cnc6ccc(Oc7c(F)ccc(NS(=O)(=O)C8CCCC8)c7C#N)cc6n4)C5)CC3)cc21] Compound 270 Stereoisomer 1 | Compound 270 |
| 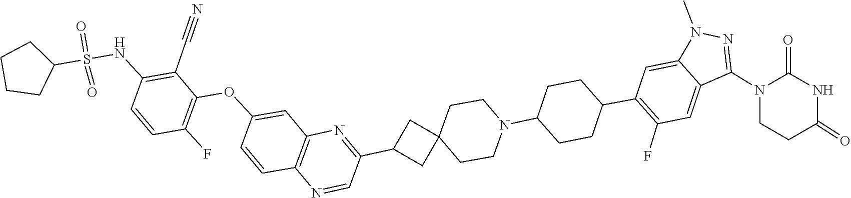 [SMILES: Cn1nc(N2CCC(=O)NC2=O)c2cc(F)c(C3CCC(N4CCC5(CC4)CC(c4cnc6ccc(Oc7c(F)ccc(NS(=O)(=O)C8CCCC8)c7C#N)cc6n4)C5)CC3)cc21] Compound 271 Stereoisomer 2 | Compound 271 |
| 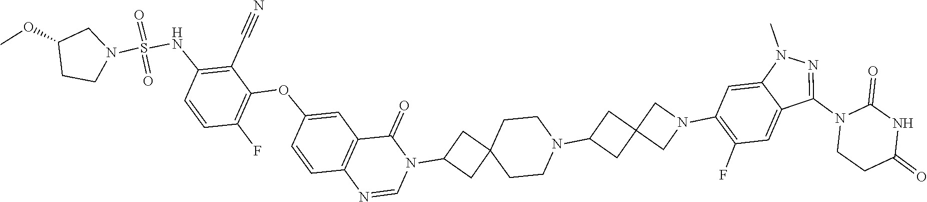 [SMILES: CO[C@H]1CCN(S(=O)(=O)Nc2ccc(F)c(Oc3ccc4ncn(C5CC6(CCN(C7CC8(C7)CN(c7cc9c(cc7F)c(N7CCC(=O)NC7=O)nn9C)C8)CC6)C5)c(=O)c4c3)c2C#N)C1] Compound 272 | Compound 272 |
| 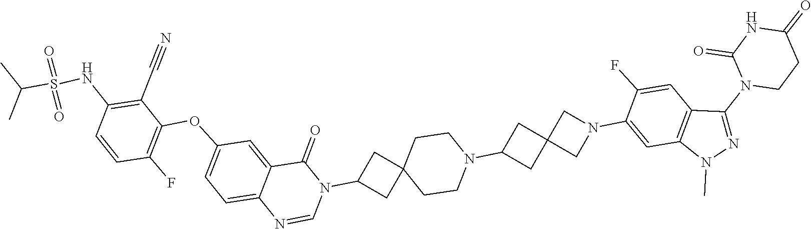 [SMILES: CC(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)CC5)C4)c(=O)c3c2)c1C#N] Compound 273 | Compound 273 |
| 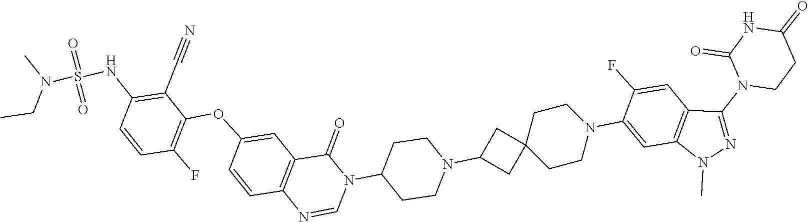 [SMILES: CCN(C)S(=O)(=O)Nc1ccc(F)c(Oc2ccc3ncn(C4CCN(C5CC6(CCN(c7cc8c(cc7F)c(N7CCC(=O)NC7=O)nn8C)CC6)C5)CC4)c(=O)c3c2)c1C#N] Compound 274 | Compound 274 |
| 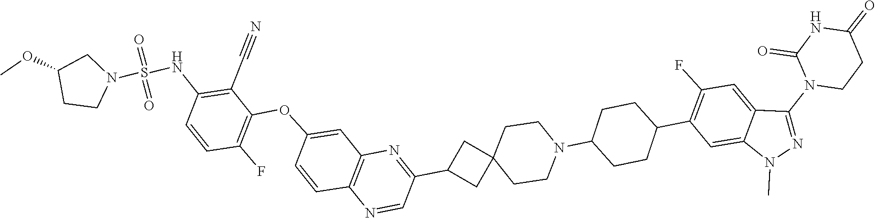 [SMILES: CO[C@H]1CCN(S(=O)(=O)Nc2ccc(F)c(Oc3ccc4ncc(C5CC6(CCN(C7CCC(c8cc9c(cc8F)c(N8CCC(=O)NC8=O)nn9C)CC7)CC6)C5)nc4c3)c2C#N)C1] Compound 275 Stereoisomer 1 | Compound 275 |
| 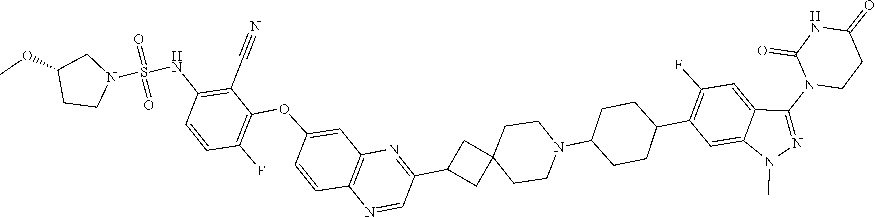 [SMILES: CO[C@H]1CCN(S(=O)(=O)Nc2ccc(F)c(Oc3ccc4ncc(C5CC6(CCN(C7CCC(c8cc9c(cc8F)c(N8CCC(=O)NC8=O)nn9C)CC7)CC6)C5)nc4c3)c2C#N)C1] Compound 276 Stereoisomer 2 | Compound 276 |
| 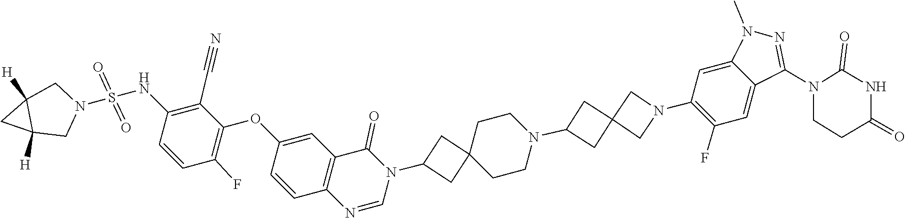 [SMILES: Cn1nc(N2CCC(=O)NC2=O)c2cc(F)c(N3CC4(CC(N5CCC6(CC5)CC(n5cnc7ccc(Oc8c(F)ccc(NS(=O)(=O)N9C[C@H]%10C[C@H]%10C9)c8C#N)cc7c5=O)C6)C4)C3)cc21] Compound 277 | Compound 277 |
| 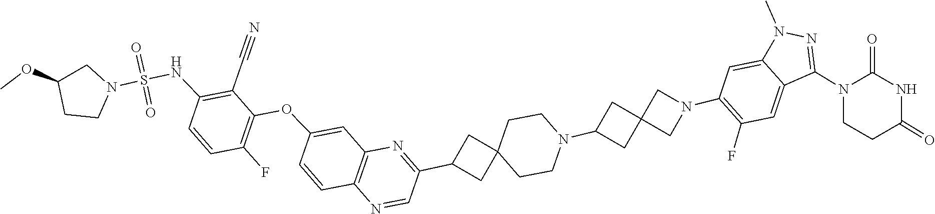 [SMILES: CO[C@@H]1CCN(S(=O)(=O)Nc2ccc(F)c(Oc3ccc4ncc(C5CC6(CCN(C7CC8(C7)CN(c7cc9c(cc7F)c(N7CCC(=O)NC7=O)nn9C)C8)CC6)C5)nc4c3)c2C#N)C1] Compound 278 | Compound 278 |
| 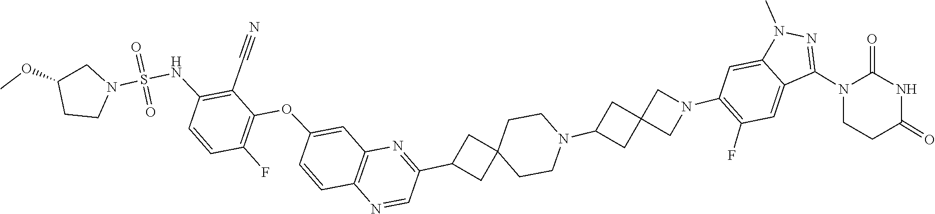 [SMILES: CO[C@H]1CCN(S(=O)(=O)Nc2ccc(F)c(Oc3ccc4ncc(C5CC6(CCN(C7CC8(C7)CN(c7cc9c(cc7F)c(N7CCC(=O)NC7=O)nn9C)C8)CC6)C5)nc4c3)c2C#N)C1] Compound 279 | Compound 279 |
| 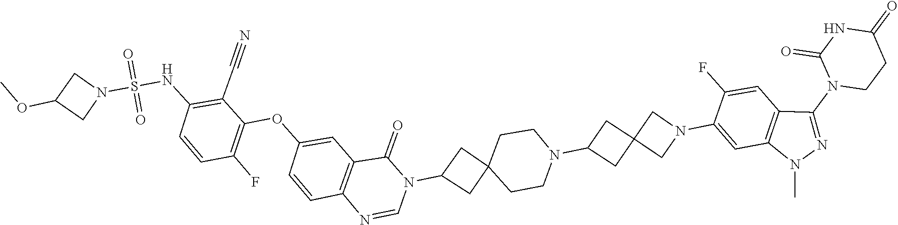 [SMILES: COC1CN(S(=O)(=O)Nc2ccc(F)c(Oc3ccc4ncn(C5CC6(CCN(C7CC8(C7)CN(c7cc9c(cc7F)c(N7CCC(=O)NC7=O)nn9C)C8)CC6)C5)c(=O)c4c3)c2C#N)C1] Compound 280 | Compound 280 |
| 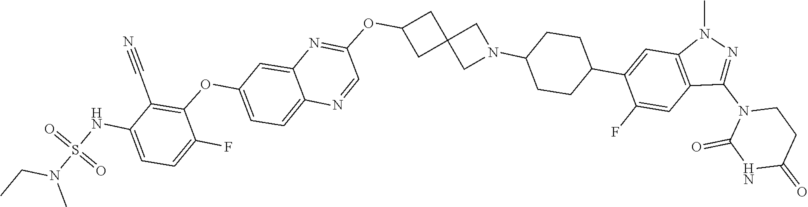 [SMILES: CCN(C)S(=O)(=O)Nc1ccc(F)c(Oc2ccc3ncc(OC4CC5(C4)CN(C4CCC(c6cc7c(cc6F)c(N6CCC(=O)NC6=O)nn7C)CC4)C5)nc3c2)c1C#N] Compound 281 Stereoisomer 1 | 281 |
| 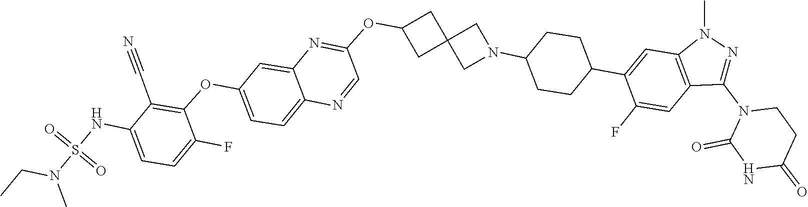 [SMILES: CCN(C)S(=O)(=O)Nc1ccc(F)c(Oc2ccc3ncc(OC4CC5(C4)CN(C4CCC(c6cc7c(cc6F)c(N6CCC(=O)NC6=O)nn7C)CC4)C5)nc3c2)c1C#N] Compound 282 Stereoisomer 2 | Compound 282 |
| 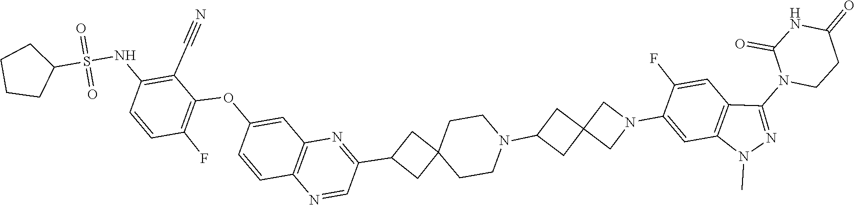 [SMILES: Cn1nc(N2CCC(=O)NC2=O)c2cc(F)c(N3CC4(CC(N5CCC6(CC5)CC(c5cnc7ccc(Oc8c(F)ccc(NS(=O)(=O)C9CCCC9)c8C#N)cc7n5)C6)C4)C3)cc21] Compound 283 | Compound 283 |
| 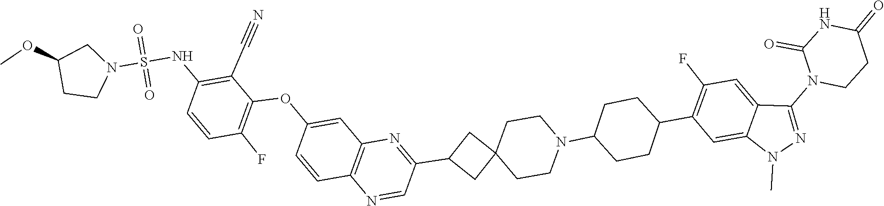 [SMILES: CO[C@@H]1CCN(S(=O)(=O)Nc2ccc(F)c(Oc3ccc4ncc(C5CC6(CCN(C7CCC(c8cc9c(cc8F)c(N8CCC(=O)NC8=O)nn9C)CC7)CC6)C5)nc4c3)c2C#N)C1] Compound 284 Stereoisomer 1 | Compound 284 |
| 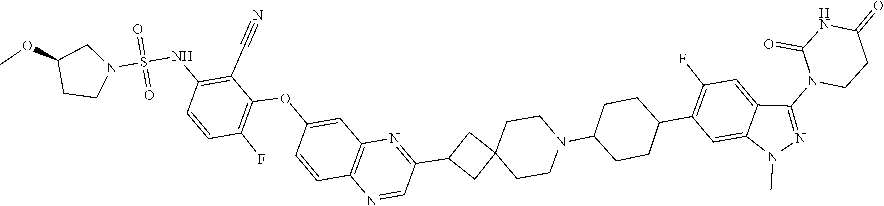 [SMILES: CO[C@@H]1CCN(S(=O)(=O)Nc2ccc(F)c(Oc3ccc4ncc(C5CC6(CCN(C7CCC(c8cc9c(cc8F)c(N8CCC(=O)NC8=O)nn9C)CC7)CC6)C5)nc4c3)c2C#N)C1] Compound 285 Stereoisomer 2 | Compound 285 |
| 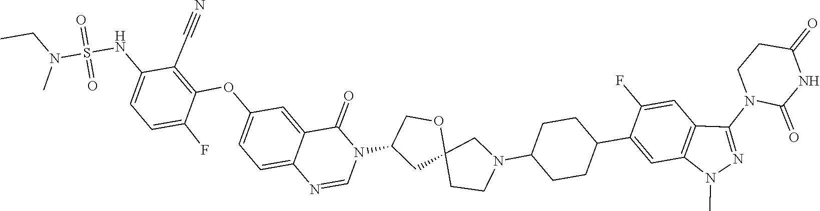 [SMILES: CCN(C)S(=O)(=O)Nc1ccc(F)c(Oc2ccc3ncn([C@@H]4CO[C@@]5(CCN(C6CCC(c7cc8c(cc7F)c(N7CCC(=O)NC7=O)nn8C)CC6)C5)C4)c(=O)c3c2)c1C#N] Compound 286 Stereoisomer 1 | Compound 286 |
| 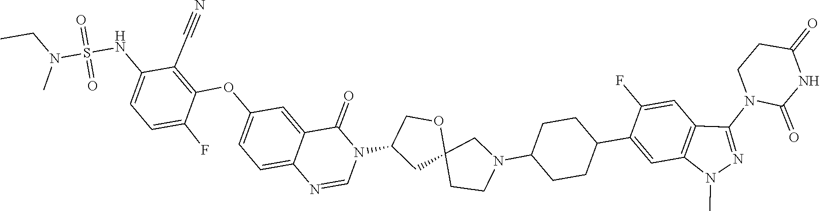 [SMILES: CCN(C)S(=O)(=O)Nc1ccc(F)c(Oc2ccc3ncn([C@@H]4CO[C@@]5(CCN(C6CCC(c7cc8c(cc7F)c(N7CCC(=O)NC7=O)nn8C)CC6)C5)C4)c(=O)c3c2)c1C#N] Compound 287 Stereoisomer 2 | Compound 287 |
| 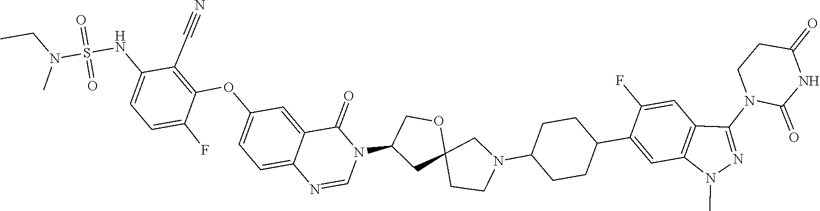 [SMILES: CCN(C)S(=O)(=O)Nc1ccc(F)c(Oc2ccc3ncn([C@H]4CO[C@]5(CCN(C6CCC(c7cc8c(cc7F)c(N7CCC(=O)NC7=O)nn8C)CC6)C5)C4)c(=O)c3c2)c1C#N] Compound 288 Stereoisomer 1 | Compound 288 |
| 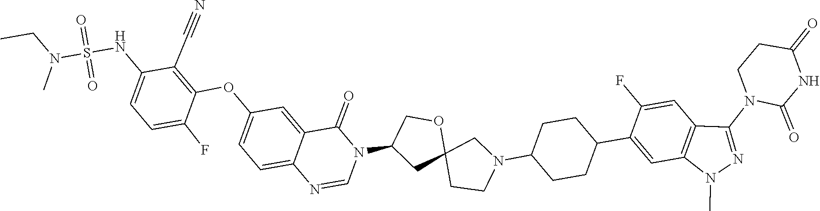 [SMILES: CCN(C)S(=O)(=O)Nc1ccc(F)c(Oc2ccc3ncn([C@H]4CO[C@]5(CCN(C6CCC(c7cc8c(cc7F)c(N7CCC(=O)NC7=O)nn8C)CC6)C5)C4)c(=O)c3c2)c1C#N] Compound 289 Stereoisomer 2 | Compound 289 |
| 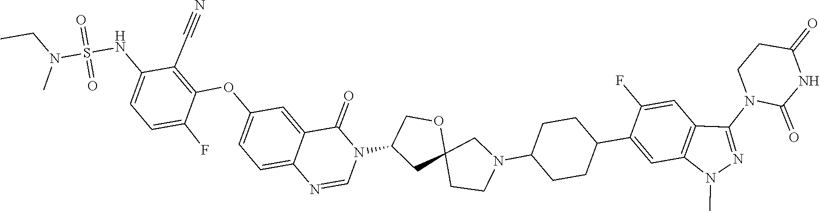 [SMILES: CCN(C)S(=O)(=O)Nc1ccc(F)c(Oc2ccc3ncn([C@@H]4CO[C@]5(CCN(C6CCC(c7cc8c(cc7F)c(N7CCC(=O)NC7=O)nn8C)CC6)C5)C4)c(=O)c3c2)c1C#N] Compound 290 Stereoisomer 1 | Compound 290 |
| 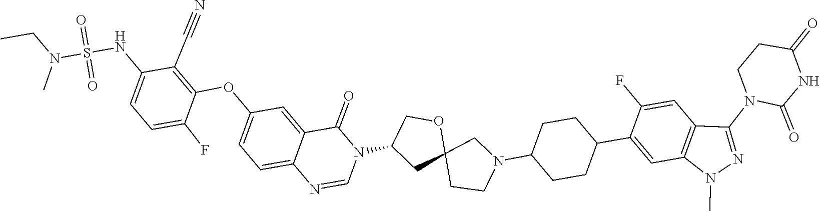 [SMILES: CCN(C)S(=O)(=O)Nc1ccc(F)c(Oc2ccc3ncn([C@@H]4CO[C@]5(CCN(C6CCC(c7cc8c(cc7F)c(N7CCC(=O)NC7=O)nn8C)CC6)C5)C4)c(=O)c3c2)c1C#N] Compound 291 Stereoisomer 2 | Compound 291 |
| 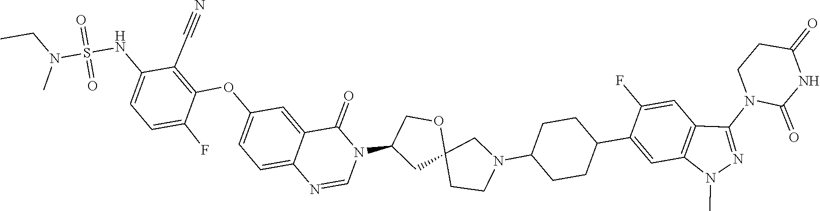 [SMILES: CCN(C)S(=O)(=O)Nc1ccc(F)c(Oc2ccc3ncn([C@H]4CO[C@@]5(CCN(C6CCC(c7cc8c(cc7F)c(N7CCC(=O)NC7=O)nn8C)CC6)C5)C4)c(=O)c3c2)c1C#N] Compound 292 Stereoisomer 1 | Compound 292 |
| 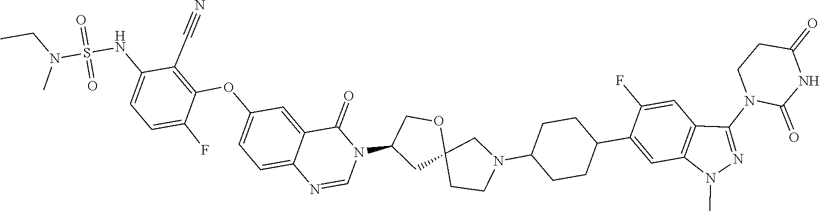 [SMILES: CCN(C)S(=O)(=O)Nc1ccc(F)c(Oc2ccc3ncn([C@H]4CO[C@@]5(CCN(C6CCC(c7cc8c(cc7F)c(N7CCC(=O)NC7=O)nn8C)CC6)C5)C4)c(=O)c3c2)c1C#N] Compound 293 Stereoisomer 2 | Compound 293 |
| 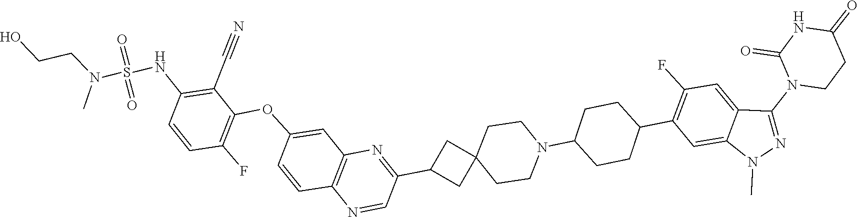 [SMILES: CN(CCO)S(=O)(=O)Nc1ccc(F)c(Oc2ccc3ncc(C4CC5(CCN(C6CCC(c7cc8c(cc7F)c(N7CCC(=O)NC7=O)nn8C)CC6)CC5)C4)nc3c2)c1C#N] Compound 294 Stereoisomer 1 | Compound 294 |
| 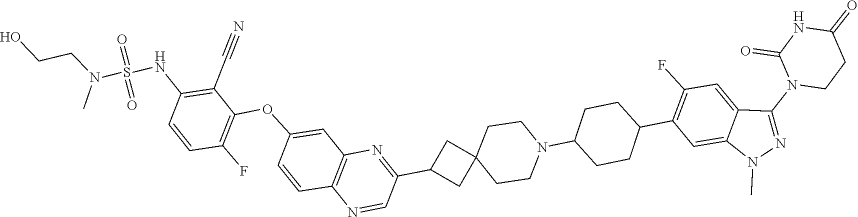 [SMILES: CN(CCO)S(=O)(=O)Nc1ccc(F)c(Oc2ccc3ncc(C4CC5(CCN(C6CCC(c7cc8c(cc7F)c(N7CCC(=O)NC7=O)nn8C)CC6)CC5)C4)nc3c2)c1C#N] Compound 295 Stereoisomer 2 | Compound 295 |
| 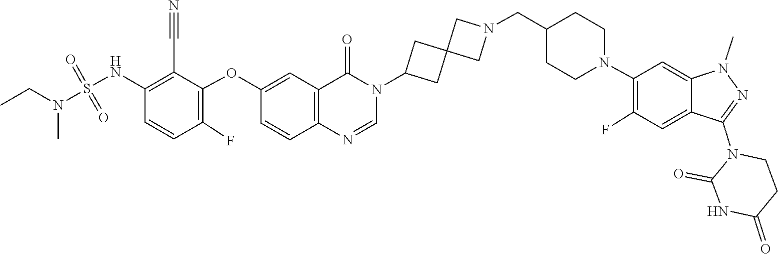 [SMILES: CCN(C)S(=O)(=O)Nc1ccc(F)c(Oc2ccc3ncn(C4CC5(C4)CN(CC4CCN(c6cc7c(cc6F)c(N6CCC(=O)NC6=O)nn7C)CC4)C5)c(=O)c3c2)c1C#N] Compound 296 | Compound 296 |
| 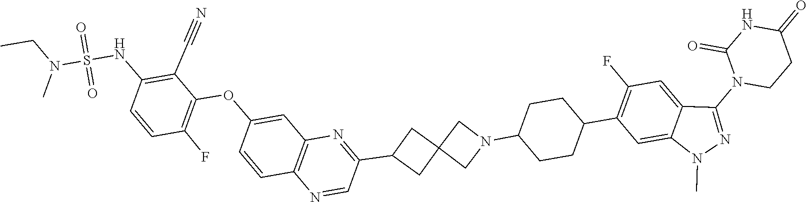 [SMILES: CCN(C)S(=O)(=O)Nc1ccc(F)c(Oc2ccc3ncc(C4CC5(C4)CN(C4CCC(c6cc7c(cc6F)c(N6CCC(=O)NC6=O)nn7C)CC4)C5)nc3c2)c1C#N] Compound 297 Stereoisomer 1 | Compound 297 |
| 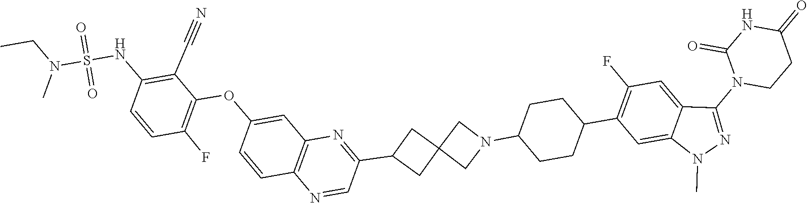 [SMILES: CCN(C)S(=O)(=O)Nc1ccc(F)c(Oc2ccc3ncc(C4CC5(C4)CN(C4CCC(c6cc7c(cc6F)c(N6CCC(=O)NC6=O)nn7C)CC4)C5)nc3c2)c1C#N] Compound 298 Stereoisomer 2 | Compound 298 |
| 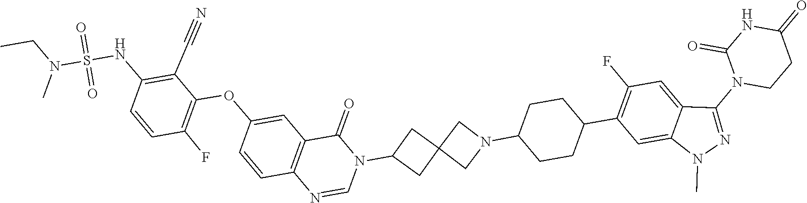 [SMILES: CCN(C)S(=O)(=O)Nc1ccc(F)c(Oc2ccc3ncn(C4CC5(C4)CN(C4CCC(c6cc7c(cc6F)c(N6CCC(=O)NC6=O)nn7C)CC4)C5)c(=O)c3c2)c1C#N] Compound 299 Stereoisomer 1 | Compound 299 |
| 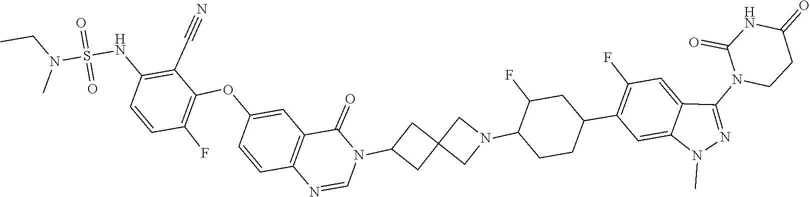 [SMILES: CCN(C)S(=O)(=O)Nc1ccc(F)c(Oc2ccc3ncn(C4CC5(C4)CN(C4CCC(c6cc7c(cc6F)c(N6CCC(=O)NC6=O)nn7C)CC4F)C5)c(=O)c3c2)c1C#N] Compound 300 Stereoisomer 2 | Compound 300 |
| 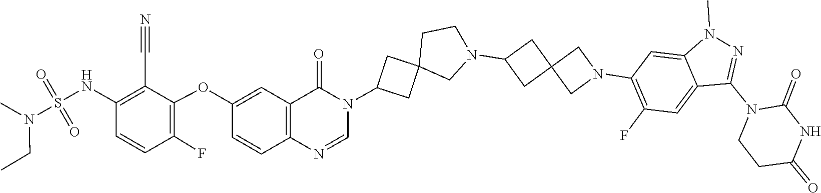 [SMILES: CCN(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)C5)C4)c(=O)c3c2)c1C#N] Compound 301 Stereoisomer 1 | Compound 301 |
| 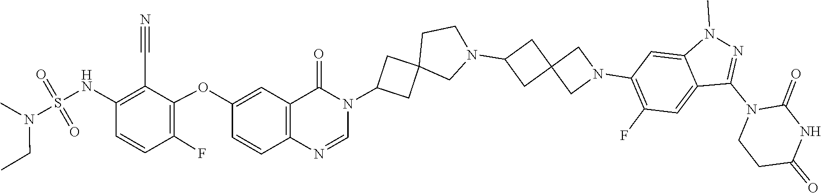 [SMILES: CCN(C)S(=O)(=O)Nc1ccc(F)c(Oc2ccc3ncn(C4CC5(CCN(C6CC7(C6)CN(c6cc8c(cc6F)c(N6CCC(=O)NC6=O)nn8C)C7)C5)C4)c(=O)c3c2)c1C#N] Compound 302 Stereoisomer 2 | Compound 302 |
| 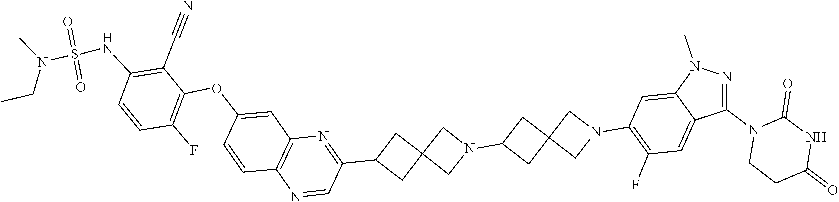 [SMILES: CCN(C)S(=O)(=O)Nc1ccc(F)c(Oc2ccc3ncc(C4CC5(C4)CN(C4CC6(C4)CN(c4cc7c(cc4F)c(N4CCC(=O)NC4=O)nn7C)C6)C5)nc3c2)c1C#N] Compound 303 | Compound 303 |
| 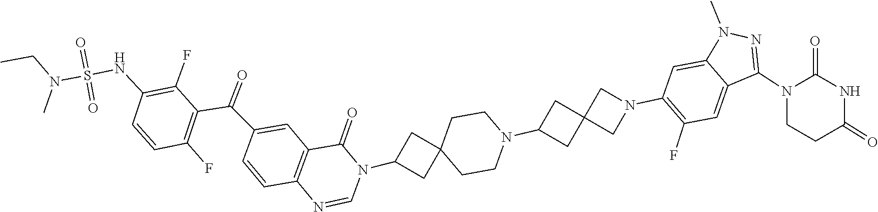 [SMILES: CCN(C)S(=O)(=O)Nc1ccc(F)c(C(=O)c2ccc3ncn(C4CC5(CCN(C6CC7(C6)CN(c6cc8c(cc6F)c(N6CCC(=O)NC6=O)nn8C)C7)CC5)C4)c(=O)c3c2)c1F] Compound 304 | Compound 304 |
| 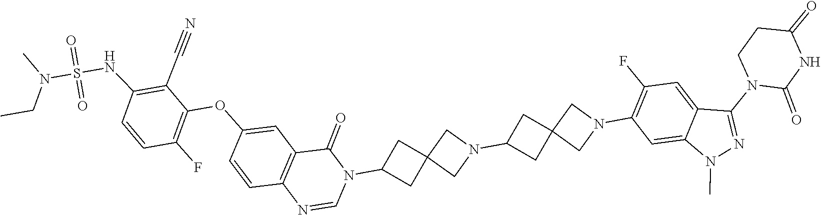 [SMILES: CCN(C)S(=O)(=O)Nc1ccc(F)c(Oc2ccc3ncn(C4CC5(C4)CN(C4CC6(C4)CN(c4cc7c(cc4F)c(N4CCC(=O)NC4=O)nn7C)C6)C5)c(=O)c3c2)c1C#N] Compound 305 | Compound 305 |
| 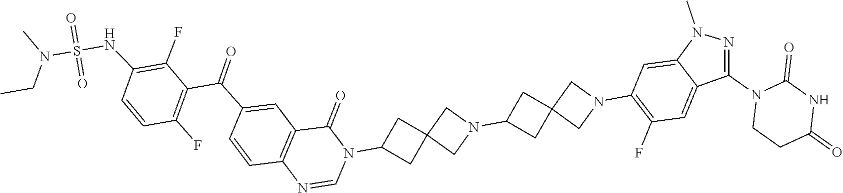 [SMILES: CCN(C)S(=O)(=O)Nc1ccc(F)c(C(=O)c2ccc3ncn(C4CC5(C4)CN(C4CC6(C4)CN(c4cc7c(cc4F)c(N4CCC(=O)NC4=O)nn7C)C6)C5)c(=O)c3c2)c1F] Compound 306 | Compound 306 |
| 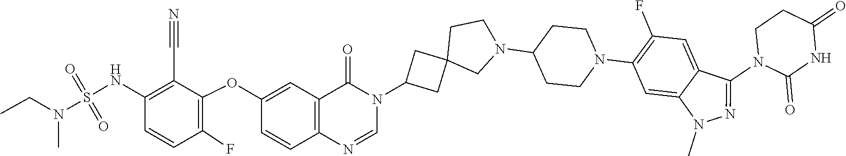 [SMILES: CCN(C)S(=O)(=O)Nc1ccc(F)c(Oc2ccc3ncn(C4CC5(CCN(C6CCN(c7cc8c(cc7F)c(N7CCC(=O)NC7=O)nn8C)CC6)C5)C4)c(=O)c3c2)c1C#N] Compound 307 Stereoisomer 1 | Compound 307 |
| 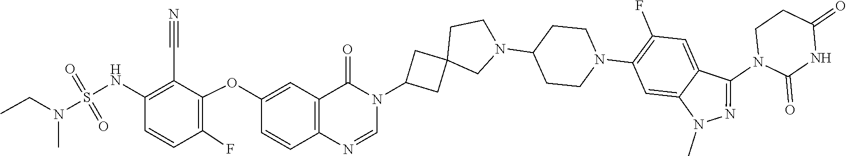 [SMILES: CCN(C)S(=O)(=O)Nc1ccc(F)c(Oc2ccc3ncn(C4CC5(CCN(C6CCN(c7cc8c(cc7F)c(N7CCC(=O)NC7=O)nn8C)CC6)C5)C4)c(=O)c3c2)c1C#N] Compound 308 Stereoisomer 2 | Compound 308 |
| 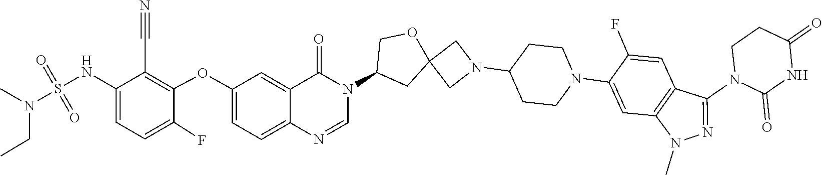 [SMILES: CCN(C)S(=O)(=O)Nc1ccc(F)c(Oc2ccc3ncn([C@H]4COC5(C4)CN(C4CCN(c6cc7c(cc6F)c(N6CCC(=O)NC6=O)nn7C)CC4)C5)c(=O)c3c2)c1C#N] Compound 309 | Compound 309 |
| 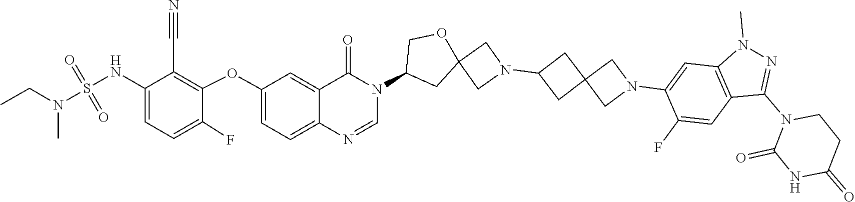 [SMILES: CCN(C)S(=O)(=O)Nc1ccc(F)c(Oc2ccc3ncn([C@H]4COC5(C4)CN(C4CC6(C4)CN(c4cc7c(cc4F)c(N4CCC(=O)NC4=O)nn7C)C6)C5)c(=O)c3c2)c1C#N] Compound 310 | Compound 310 |
| 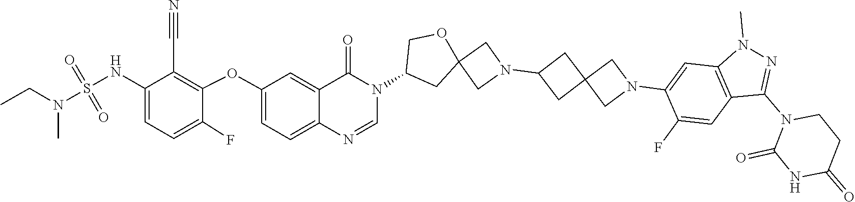 [SMILES: CCN(C)S(=O)(=O)Nc1ccc(F)c(Oc2ccc3ncn([C@@H]4COC5(C4)CN(C4CC6(C4)CN(c4cc7c(cc4F)c(N4CCC(=O)NC4=O)nn7C)C6)C5)c(=O)c3c2)c1C#N] Compound 311 | Compound 311 |
| 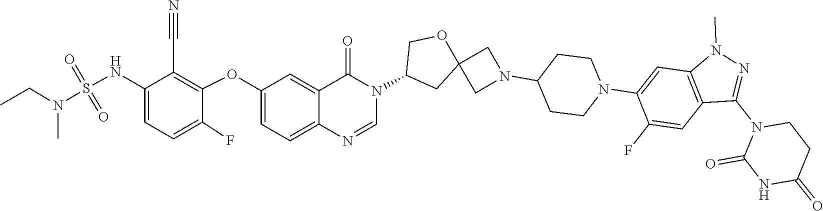 [SMILES: CCN(C)S(=O)(=O)Nc1ccc(F)c(Oc2ccc3ncn([C@@H]4COC5(C4)CN(C4CCN(c6cc7c(cc6F)c(N6CCC(=O)NC6=O)nn7C)CC4)C5)c(=O)c3c2)c1C#N] Compound 312 | Compound 312 |
| 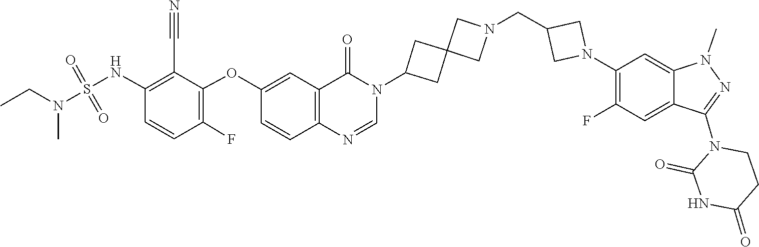 [SMILES: CCN(C)S(=O)(=O)Nc1ccc(F)c(Oc2ccc3ncn(C4CC5(C4)CN(CC4CN(c6cc7c(cc6F)c(N6CCC(=O)NC6=O)nn7C)C4)C5)c(=O)c3c2)c1C#N] Compound 313 | Compound 313 |
| 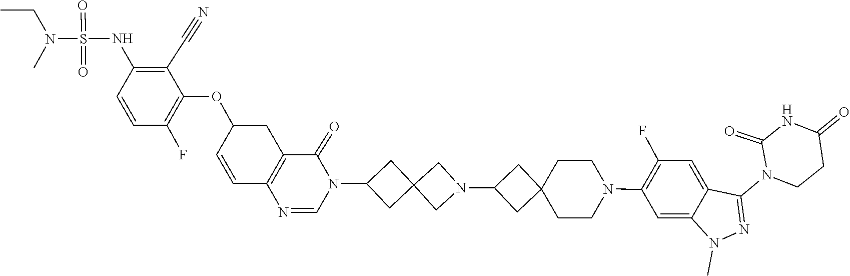 [SMILES: CCN(C)S(=O)(=O)Nc1ccc(F)c(OC2C=Cc3ncn(C4CC5(C4)CN(C4CC6(CCN(c7cc8c(cc7F)c(N7CCC(=O)NC7=O)nn8C)CC6)C4)C5)c(=O)c3C2)c1C#N] Compound 314 | Compound 314 |
| 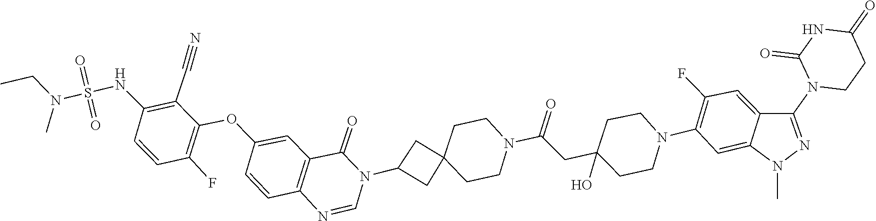 [SMILES: CCN(C)S(=O)(=O)Nc1ccc(F)c(Oc2ccc3ncn(C4CC5(CCN(C(=O)CC6(O)CCN(c7cc8c(cc7F)c(N7CCC(=O)NC7=O)nn8C)CC6)CC5)C4)c(=O)c3c2)c1C#N] Compound 315 | Compound 315 |
| 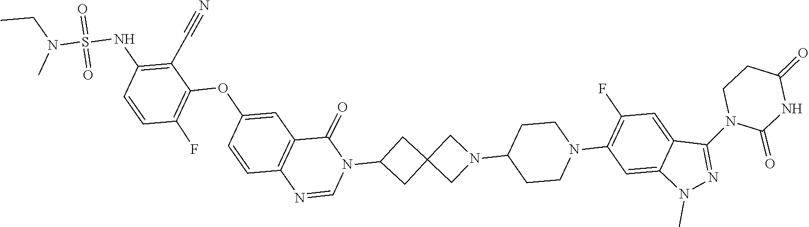 [SMILES: CCN(C)S(=O)(=O)Nc1ccc(F)c(Oc2ccc3ncn(C4CC5(C4)CN(C4CCN(c6cc7c(cc6F)c(N6CCC(=O)NC6=O)nn7C)CC4)C5)c(=O)c3c2)c1C#N] Compound 316 | Compound 316 |
| 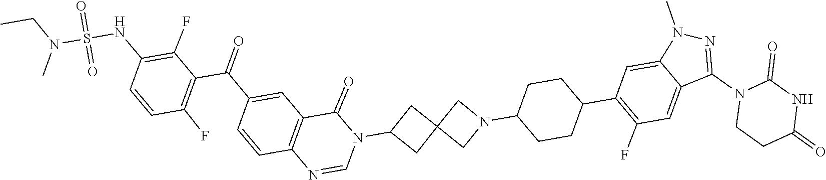 [SMILES: CCN(C)S(=O)(=O)Nc1ccc(F)c(C(=O)c2ccc3ncn(C4CC5(C4)CN(C4CCC(c6cc7c(cc6F)c(N6CCC(=O)NC6=O)nn7C)CC4)C5)c(=O)c3c2)c1F] Compound 317 Stereoisomer 1 | Compound 317 |
| 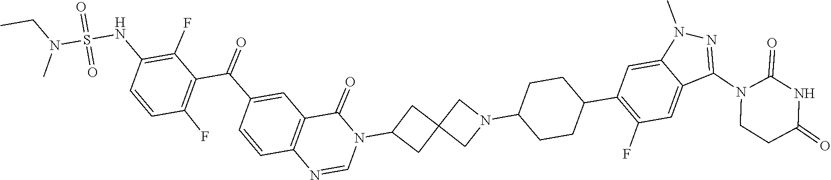 [SMILES: CCN(C)S(=O)(=O)Nc1ccc(F)c(C(=O)c2ccc3ncn(C4CC5(C4)CN(C4CCC(c6cc7c(cc6F)c(N6CCC(=O)NC6=O)nn7C)CC4)C5)c(=O)c3c2)c1F] Compound 318 Stereoisomer 2 | Compound 318 |
| 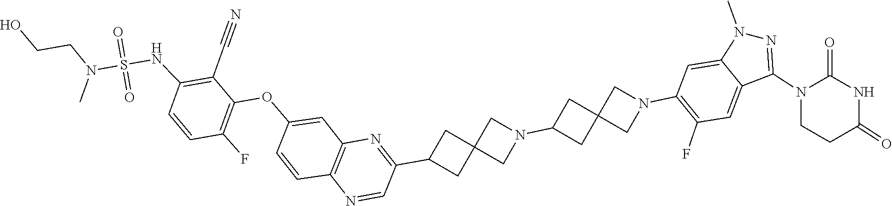 [SMILES: CN(CCO)S(=O)(=O)Nc1ccc(F)c(Oc2ccc3ncc(C4CC5(C4)CN(C4CC6(C4)CN(c4cc7c(cc4F)c(N4CCC(=O)NC4=O)nn7C)C6)C5)nc3c2)c1C#N] Compound 319 | Compound 319 |
| 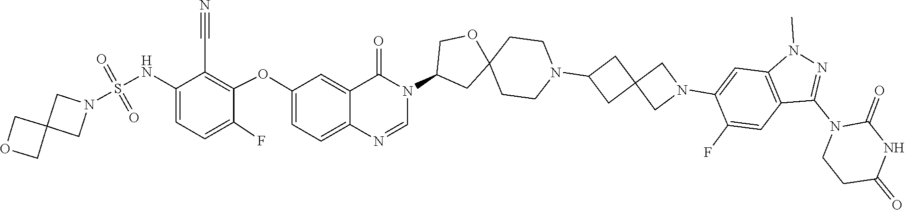 [SMILES: Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)N9CC%10(COC%10)C9)c8C#N)cc7c5=O)CO6)C4)C3)cc21] Compound 320 | Compound 320 |
| 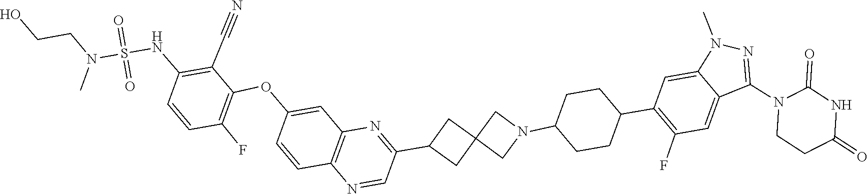 [SMILES: CN(CCO)S(=O)(=O)Nc1ccc(F)c(Oc2ccc3ncc(C4CC5(C4)CN(C4CCC(c6cc7c(cc6F)c(N6CCC(=O)NC6=O)nn7C)CC4)C5)nc3c2)c1C#N] Compound 321 Stereoisomer 1 | Compound 321 |
| 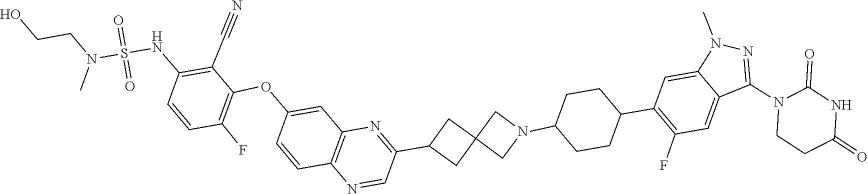 [SMILES: CN(CCO)S(=O)(=O)Nc1ccc(F)c(Oc2ccc3ncc(C4CC5(C4)CN(C4CCC(c6cc7c(cc6F)c(N6CCC(=O)NC6=O)nn7C)CC4)C5)nc3c2)c1C#N] Compound 322 Stereoisomer 2 | Compound 322 |

### Example 5 Cellular HiBiT Assays and Cell Viability BRAF V600E HiBiT Degradation Assay

Compounds of this invention were tested for degradation of mutant BRAF V600E using the HiBiT assay, a luminescence-based assay for quantification of cellular protein levels using the Nano-Glo® HiBiT Lytic Detection System (Catalog No. N3050, Promega, Madison, WI USA). This assay utilizes the HiBiT tag on a protein of interest, which creates a luminescent signal in the presence of a complementing peptide and its substrate, the quantification of which is proportional to the amount of HiBiT-tagged protein in the cell.

The cell line A375, which is homozygous for the BRAF V600E mutation, was purchased from ATCC (Catalog No. CRL-1619, Manassas, VA, USA). A375 cells were engineered to express HiBiT tagged BRAF V600E by CRISPR knock-in of the HiBiT tag to the endogenous BRAF V600E locus, followed by selection of a clonal population containing the appropriate knock-in (referred to here as cell line A375.10). A375.10 cells were maintained at 37° C. in an atmosphere of 5% CO₂ in Dulbecco's modified Eagle Medium (DMEM) with high glucose (Catalog No. 11965092), supplemented with 10% fetal bovine serum (FBS; Catalog No. 10437036), and 10 mM HEPES (Catalog No. 15630130), all obtained from Thermofisher Scientific (Waltham, MA, USA). Cell culture flasks and 384-well microplates were acquired from VWR (Radnor, PA, USA).

Test compounds were prepared by dissolving neat compounds in DMSO (Catalog No. D8418, Sigma-Aldrich, Inc., St. Louis, MO, USA) to generate 10 mM stock solutions, which were serially diluted (half log) in DMSO to generate an 11-point dose series (1000× final treatment concentration) in a 384-Well Low Dead Volume Microplate (Catalog No. LP-0200, Labcyte, San Jose, CA, USA). Using the Echo 550 Acoustic Liquid Handler (Labcyte), 30 nL of serially diluted compound solutions were dispensed in duplicate to 384-well TC-treated microplates (Catalog No. 3571, Corning, Glendale, CA, USA) and 30 nL of DMSO was dispensed to control wells. A375.10 cells resuspended in growth media without phenol red (Catalog No. 21063-029, Thermofisher Scientific) were then added to 384-well plates containing the 11-point dose series of compounds in duplicate (maximum 10 μM concentration for final treatment) at a density of 12,000 cells/well in 30 μl. Plates were then spun briefly, and cells were incubated with compound at 37° C. for 2 hours, 6 hours, or 24 hours.

Following the appropriate treatment time, Nano-Glo® HiBiT Lytic Assay reagents were added to the cells per the manufacturer's instructions by adding 30 μl of reagent per well, and the luminescence signal was acquired by plate reader. Cells treated with DMSO only (no test compound treatment) were used as the negative control (N). Media only (no cells added) with Nano-Glo reagent addition was used as the background or positive control (P). The percent response of compound treated samples were calculated by subtracting out the background signal (P) and normalizing to the DMSO control (N) as follows.

% Response=100×(Treated Signal−Average (P))/(Average (N)−Average (P)).

Data were analyzed with a 4-parameter logistic fit to generate sigmoidal dose-response curves. The DC₅₀ is the concentration of compound at which 50% of the cellular BRAF V600E has been degraded. The Emax, or maximum effect of each compound, represents the amount of residual protein remaining in the cell following compound treatment. The IP, or inflection point of the dose-response curve, is the concentration of compound at which 50% of the observed degradation response is achieved.

The experiment was conducted at least once for each compound. If the experiment was conducted multiple times, the data shown in the table is the average of the two or more experiments.

### CTGlo Cell Viability Assay

Compounds of this invention were tested for their effect on the viability of A375-NRAS^{Q61K} cells by quantification of ATP using the CellTiter-Glo® 2.0 Cell Viability Assay (Catalog No. G9243, Promega, Madison, WI, USA). This assay produces a luminescent signal in the presence of ATP, which is indicative of metabolically active cells, making the luminescence readout proportional to the number of viable cells.

A375-NRAS^{Q61K} cells (harboring BRAF homozygous V600E mutation and NRAS homozygous Q61K mutation) were purchased from ATCC (Catalog No. CRL-1619IG-2, Manassas, VA, USA). A375-NRAS^{Q61K} cells (also referred to here as A375.11) were maintained at 37° C. in an atmosphere of 5% CO₂ in Dulbecco's modified Eagle Medium (DMEM) with high glucose (Catalog No. 11965092), supplemented with 10% fetal bovine serum (FBS; Catalog No. 10437036), and 10 mM HEPES (Catalog No. 15630130), all obtained from Thermofisher Scientific (Waltham, MA, USA). Cell culture flasks and 384-well microplates were acquired from VWR (Radnor, PA, USA).

Test compounds were prepared by dissolving neat compounds in DMSO (Catalog No. D8418, Sigma-Aldrich, Inc., St. Louis, MO, USA) to generate 10 mM stock solutions, which were serially diluted (half log) in DMSO to generate an 11-point dose series (1000× final treatment concentration) in a 384-Well Low Dead Volume Microplate (Catalog No. LP-0200, Labcyte, San Jose, CA, USA). Using the Echo 550 Acoustic Liquid Handler (Labcyte), 50 nL of serially diluted compound solutions were dispensed in duplicate to 384-well TC-treated microplates (Catalog No. 3571, Corning, Glendale, CA, USA) and 50 nL of DMSO was dispensed to control wells. A375-NRAS^{Q61K} cells were resuspended in growth media and added to 384-well plates containing a 10-point dose series of compounds in duplicate (maximum 10 μM concentration for final treatment) at a density of 250 cells/well in 50 μl. Cells were also plated to a 384-well plate with no compound treatment, to which CellTiter-Glo® reagent was added and luminescence was read right after plating to act as a baseline signal at time of plating (To). Plates treated with compound were then spun briefly, and cells were incubated with compound at 37° C. for 96 hours.

After 96 hours compound treatment, CellTiter-Glo® reagent was added to the cells at 25 μl per well and luminescence was read out on the PHERAstar plate reader (BMG Labtech, Ortenberg, Germany). Cells treated with DMSO only (no test compound treatment) were used as the negative control (N). DMSO treated cells with no CellTiter-Glo® reagent added were used as the background or positive control (P). Viability was determined by subtracting out the average background signal (P) and normalized to the average untreated signal (N) set at 100%.

Data were analyzed with a 4-parameter logistic fit to generate sigmoidal dose-response curves. The half-maximal inhibition of cell growth (GI50) is the concentration at which the fitted curve crosses 50%. The Emax, or maximum effect of each compound, represents the lowest % viability achieved following compound treatment.

TABLE 1
BRAFV600E 2 hours HiBiT

| | HiBiT- | HiBiT- | HiBiT- |
|---|---|---|---|
| | Degradation | Degradation | Degradation |
| | A375.10 | A375.10 | A375.10 |
| | BRAF | BRAF | BRAF |
| | 2 hours | 2 hours | 2 hours |
| Compound | DC₅₀ nM | Emax | IP nM |
| Compound 10 | * | ** | *** |
| Compound 100 | *** | *** | *** |
| Compound 101 | *** | *** | *** |
| Compound 102 | *** | *** | *** |
| Compound 103 | *** | *** | *** |
| Compound 104 | *** | *** | *** |
| Compound 106 | ** | *** | *** |
| Compound 109 | *** | *** | *** |
| Compound 11 | *** | *** | *** |
| Compound 110 | *** | ** | *** |
| Compound 113 | *** | *** | *** |
| Compound 115 | *** | *** | *** |
| Compound 116 | ** | ** | *** |
| Compound 117 | *** | *** | *** |
| Compound 118 | *** | ** | *** |
| Compound 119 | *** | ** | *** |
| Compound 12 | *** | *** | *** |
| Compound 121 | *** | *** | *** |
| Compound 122 | *** | *** | *** |
| Compound 123 | *** | *** | *** |
| Compound 124 | ** | *** | *** |
| Compound 126 | *** | *** | *** |
| Compound 128 | * | ** | *** |
| Compound 129 | *** | ** | *** |
| Compound 13 | *** | *** | *** |
| Compound 130 | *** | ** | *** |
| Compound 131 | * | ** | *** |
| Compound 132 | *** | *** | *** |
| Compound 133 | * | ** | *** |
| Compound 134 | *** | ** | *** |
| Compound 136 | *** | *** | *** |
| Compound 137 | *** | ** | *** |
| Compound 138 | ** | ** | *** |
| Compound 140 | *** | *** | *** |
| Compound 141 | *** | *** | *** |
| Compound 143 | *** | *** | *** |
| Compound 144 | ** | ** | *** |
| Compound 145 | *** | *** | *** |
| Compound 146 | * | ** | *** |
| Compound 147 | * | ** | *** |
| Compound 149 | *** | *** | *** |
| Compound 15 | *** | ** | *** |
| Compound 151 | ** | ** | *** |
| Compound 153 | ** | ** | *** |
| Compound 155 | *** | ** | *** |
| Compound 156 | *** | *** | *** |
| Compound 157 | *** | ** | *** |
| Compound 158 | ** | ** | *** |
| Compound 16 | *** | ** | *** |
| Compound 160 | *** | *** | *** |
| Compound 161 | * | ** | *** |
| Compound 163 | *** | *** | *** |
| Compound 164 | *** | ** | *** |
| Compound 165 | *** | ** | *** |
| Compound 166 | * | ** | *** |
| Compound 167 | * | ** | *** |
| Compound 168 | *** | ** | *** |
| Compound 17 | *** | ** | *** |
| Compound 170 | * | ** | *** |
| Compound 172 | * | ** | *** |
| Compound 174 | *** | *** | *** |
| Compound 176 | *** | *** | *** |
| Compound 177 | *** | *** | *** |
| Compound 178 | *** | *** | *** |
| Compound 179 | *** | ** | *** |
| Compound 180 | *** | *** | *** |
| Compound 181 | *** | *** | *** |
| Compound 182 | ** | ** | *** |
| Compound 183 | *** | *** | *** |
| Compound 184 | *** | *** | *** |
| Compound 185 | * | ** | *** |
| Compound 186 | *** | ** | *** |
| Compound 187 | ** | ** | *** |
| Compound 188 | *** | ** | *** |
| Compound 189 | *** | ** | *** |
| Compound 190 | *** | *** | *** |
| Compound 191 | *** | *** | *** |
| Compound 192 | *** | ** | *** |
| Compound 193 | * | ** | *** |
| Compound 194 | * | ** | *** |
| Compound 195 | * | ** | *** |
| Compound 196 | *** | *** | *** |
| Compound 197 | ** | ** | *** |
| Compound 198 | *** | *** | *** |
| Compound 199 | *** | *** | *** |
| Compound 200 | *** | *** | *** |
| Compound 201 | *** | *** | *** |
| Compound 203 | *** | *** | *** |
| Compound 204 | *** | *** | *** |
| Compound 205 | * | ** | *** |
| Compound 206 | *** | *** | *** |
| Compound 208 | *** | *** | *** |
| Compound 209 | *** | ** | *** |
| Compound 21 | *** | *** | *** |
| Compound 212 | *** | *** | *** |
| Compound 214 | *** | *** | *** |
| Compound 216 | *** | *** | *** |
| Compound 217 | *** | *** | *** |
| Compound 218 | *** | *** | *** |
| Compound 219 | *** | ** | *** |
| Compound 22 | *** | ** | *** |
| Compound 220 | * | ** | *** |
| Compound 221 | *** | *** | *** |
| Compound 222 | *** | *** | *** |
| Compound 223 | *** | *** | *** |
| Compound 224 | *** | *** | *** |
| Compound 225 | *** | *** | *** |
| Compound 226 | *** | *** | *** |
| Compound 227 | *** | *** | *** |
| Compound 228 | ** | ** | *** |
| Compound 229 | *** | *** | *** |
| Compound 230 | *** | *** | *** |
| Compound 231 | *** | ** | *** |
| Compound 232 | *** | ** | *** |
| Compound 233 | *** | ** | *** |
| Compound 234 | *** | *** | *** |
| Compound 235 | ** | *** | *** |
| Compound 236 | *** | *** | *** |
| Compound 237 | *** | *** | *** |
| Compound 238 | *** | *** | *** |
| Compound 24 | *** | *** | *** |
| Compound 240 | *** | *** | *** |
| Compound 241 | *** | *** | *** |
| Compound 242 | ** | ** | *** |
| Compound 243 | ** | ** | *** |
| Compound 245 | *** | ** | *** |
| Compound 246 | ** | ** | *** |
| Compound 247 | *** | *** | *** |
| Compound 248 | *** | *** | *** |
| Compound 249 | *** | *** | *** |
| Compound 25 | ** | ** | *** |
| Compound 250 | *** | *** | *** |
| Compound 251 | *** | ** | *** |
| Compound 252 | * | ** | *** |
| Compound 253 | * | ** | *** |
| Compound 254 | *** | ** | *** |
| Compound 255 | *** | ** | *** |
| Compound 256 | *** | *** | *** |
| Compound 258 | ** | ** | *** |
| Compound 259 | *** | *** | *** |
| Compound 26 | *** | *** | *** |
| Compound 260 | *** | *** | *** |
| Compound 261 | *** | *** | *** |
| Compound 262 | *** | *** | *** |
| Compound 263 | *** | *** | *** |
| Compound 264 | *** | *** | *** |
| Compound 265 | *** | *** | *** |
| Compound 266 | *** | ** | *** |
| Compound 267 | *** | *** | *** |
| Compound 268 | *** | *** | *** |
| Compound 269 | *** | *** | *** |
| Compound 27 | *** | *** | *** |
| Compound 270 | *** | *** | *** |
| Compound 271 | *** | *** | *** |
| Compound 272 | *** | *** | *** |
| Compound 273 | *** | *** | *** |
| Compound 274 | *** | *** | *** |
| Compound 275 | *** | ** | *** |
| Compound 276 | *** | ** | *** |
| Compound 277 | *** | *** | *** |
| Compound 278 | *** | *** | *** |
| Compound 279 | *** | ** | *** |
| Compound 28 | *** | *** | *** |
| Compound 280 | *** | *** | *** |
| Compound 281 | * | ** | *** |
| Compound 282 | *** | ** | *** |
| Compound 283 | *** | *** | *** |
| Compound 284 | *** | *** | *** |
| Compound 285 | *** | *** | *** |
| Compound 286 | *** | *** | *** |
| Compound 287 | *** | *** | *** |
| Compound 288 | ** | ** | *** |
| Compound 289 | *** | *** | *** |
| Compound 290 | ** | ** | *** |
| Compound 291 | * | ** | *** |
| Compound 292 | *** | *** | *** |
| Compound 293 | *** | *** | *** |
| Compound 294 | *** | ** | *** |
| Compound 295 | *** | ** | *** |
| Compound 296 | *** | ** | *** |
| Compound 297 | ** | ** | *** |
| Compound 298 | *** | *** | *** |
| Compound 299 | ** | ** | *** |
| Compound 3 | *** | *** | *** |
| Compound 30 | *** | *** | *** |
| Compound 300 | *** | *** | *** |
| Compound 301 | *** | ** | *** |
| Compound 302 | *** | ** | *** |
| Compound 303 | ** | ** | *** |
| Compound 304 | ** | ** | *** |
| Compound 305 | *** | *** | *** |
| Compound 306 | *** | ** | *** |
| Compound 307 | ** | ** | *** |
| Compound 309 | * | ** | *** |
| Compound 31 | * | ** | *** |
| Compound 310 | *** | *** | *** |
| Compound 311 | ** | ** | *** |
| Compound 313 | *** | ** | *** |
| Compound 314 | *** | *** | *** |
| Compound 32 | *** | ** | *** |
| Compound 321 | ** | * | *** |
| Compound 322 | ** | ** | *** |
| Compound 33 | *** | *** | *** |
| Compound 35 | *** | *** | *** |
| Compound 38 | *** | *** | *** |
| Compound 39 | *** | *** | *** |
| Compound 4 | *** | *** | *** |
| Compound 40 | ** | ** | *** |
| Compound 41 | *** | *** | *** |
| Compound 43 | *** | ** | *** |
| Compound 45 | *** | *** | *** |
| Compound 46 | *** | *** | *** |
| Compound 47 | *** | *** | *** |
| Compound 48 | *** | *** | *** |
| Compound 50 | *** | *** | *** |
| Compound 51 | *** | ** | *** |
| Compound 52 | *** | *** | *** |
| Compound 53 | *** | *** | *** |
| Compound 54 | *** | *** | *** |
| Compound 56 | *** | *** | *** |
| Compound 57 | *** | *** | *** |
| Compound 58 | *** | *** | *** |
| Compound 59 | *** | ** | *** |
| Compound 6 | *** | *** | *** |
| Compound 60 | *** | *** | *** |
| Compound 61 | ** | ** | *** |
| Compound 62 | *** | *** | *** |
| Compound 63 | *** | *** | *** |
| Compound 64 | *** | *** | *** |
| Compound 65 | ** | ** | *** |
| Compound 67 | *** | *** | *** |
| Compound 68 | *** | *** | *** |
| Compound 69 | *** | *** | *** |
| Compound 7 | *** | *** | *** |
| Compound 70 | *** | *** | *** |
| Compound 71 | *** | *** | *** |
| Compound 73 | *** | *** | *** |
| Compound 76 | *** | *** | *** |
| Compound 78 | *** | *** | *** |
| Compound 79 | *** | *** | *** |
| Compound 8 | *** | *** | *** |
| Compound 80 | * | ** | *** |
| Compound 81 | *** | ** | *** |
| Compound 82 | ** | ** | *** |
| Compound 83 | *** | *** | *** |
| Compound 84 | *** | *** | *** |
| Compound 85 | *** | *** | *** |
| Compound 86 | *** | *** | *** |
| Compound 87 | *** | *** | *** |
| Compound 9 | *** | *** | *** |
| Compound 90 | *** | *** | *** |
| Compound 92 | *** | *** | *** |
| Compound 93 | *** | *** | *** |
| Compound 94 | *** | *** | *** |
| Compound 95 | *** | *** | *** |
| Compound 96 | *** | *** | *** |
| Compound 96 | *** | *** | *** |

In the table above for DC₅₀ and IP >=10 μM is *; <10 μM and >1 μM is **; <=1 μM is ***
For Emax >=90% is *; <90% and >50% is **; <=50% is ***

TABLE 2
BRAFV600E 6 hours HiBiT

| | HiBiT- | HiBiT- | HiBiT- |
|---|---|---|---|
| | Degradation | Degradation | Degradation |
| | A375.10 | A375.10 | A375.10 |
| | BRAF | BRAF | BRAF |
| | 6 hours | 6 hours | 6 hours |
| Compound | DC₅₀ nM | Emax | IP nM |
| Compound 6 | *** | *** | *** |
| Compound 7 | *** | *** | *** |
| Compound 9 | *** | *** | *** |
| Compound 8 | *** | *** | *** |
| Compound 21 | *** | *** | *** |
| Compound 24 | *** | *** | *** |
| Compound 26 | *** | *** | *** |
| Compound 27 | *** | *** | *** |
| Compound 28 | *** | *** | *** |
| Compound 30 | *** | *** | *** |
| Compound 33 | *** | *** | *** |
| Compound 3 | *** | *** | *** |
| Compound 4 | *** | *** | *** |
| Compound 35 | *** | *** | *** |
| Compound 38 | *** | *** | *** |
| Compound 39 | *** | *** | *** |
| Compound 40 | *** | *** | *** |
| Compound 41 | *** | *** | *** |
| Compound 43 | *** | *** | *** |
| Compound 45 | *** | *** | *** |
| Compound 46 | *** | *** | *** |
| Compound 47 | *** | *** | *** |
| Compound 48 | *** | *** | *** |
| Compound 50 | *** | *** | *** |
| Compound 52 | *** | *** | *** |
| Compound 54 | *** | *** | *** |
| Compound 57 | *** | *** | *** |
| Compound 58 | *** | *** | *** |
| Compound 60 | *** | *** | *** |
| Compound 62 | *** | *** | *** |
| Compound 63 | *** | *** | *** |
| Compound 64 | *** | *** | *** |
| Compound 67 | *** | *** | *** |
| Compound 69 | *** | *** | *** |
| Compound 70 | *** | *** | *** |
| Compound 71 | *** | *** | *** |
| Compound 73 | *** | *** | *** |
| Compound 76 | *** | *** | *** |
| Compound 78 | *** | *** | *** |
| Compound 79 | *** | *** | *** |
| Compound 81 | *** | *** | *** |
| Compound 82 | *** | *** | *** |
| Compound 83 | *** | *** | *** |
| Compound 84 | *** | *** | *** |
| Compound 87 | *** | *** | *** |
| Compound 90 | *** | *** | *** |
| Compound 92 | *** | *** | *** |
| Compound 93 | *** | *** | *** |
| Compound 94 | *** | *** | *** |
| Compound 95 | *** | *** | *** |
| Compound 96 | *** | *** | *** |
| Compound 98 | *** | *** | *** |
| Compound 100 | *** | *** | *** |
| Compound 101 | *** | *** | *** |
| Compound 102 | *** | *** | *** |
| Compound 103 | *** | *** | *** |
| Compound 104 | *** | *** | *** |
| Compound 109 | *** | *** | *** |
| Compound 110 | *** | *** | *** |
| Compound 113 | *** | *** | *** |
| Compound 115 | *** | *** | *** |
| Compound 118 | *** | *** | *** |
| Compound 121 | *** | *** | *** |
| Compound 122 | *** | *** | *** |
| Compound 123 | *** | *** | *** |
| Compound 126 | *** | *** | *** |
| Compound 133 | *** | *** | *** |
| Compound 134 | *** | *** | *** |
| Compound 141 | *** | *** | *** |
| Compound 144 | *** | *** | *** |
| Compound 145 | *** | *** | *** |
| Compound 151 | *** | *** | *** |
| Compound 156 | *** | *** | *** |
| Compound 157 | *** | *** | *** |
| Compound 177 | *** | *** | *** |
| Compound 178 | *** | *** | *** |
| Compound 180 | *** | *** | *** |
| Compound 182 | *** | *** | *** |
| Compound 184 | *** | *** | *** |

In the table above for DC₅₀ and IP >=10 μM is *; <10 μM and >1 μM is **; <=1 μM is ***
For Emax >=90% is *; <90% and >50% is **; <=50% is ***

TABLE 3
BRAFV600E 24 hours HiBiT

| | HiBiT- | HiBiT- | HiBiT- |
|---|---|---|---|
| | Degradation | Degradation | Degradation |
| | A375.10 | A375.10 | A375.10 |
| | BRAF | BRAF | BRAF |
| | 24 hours | 24 hours | 24 hours |
| Compound | DC₅₀ nM | Emax | IP nM |
| Compound 6 | *** | *** | *** |
| Compound 7 | *** | *** | *** |
| Compound 9 | *** | *** | *** |
| Compound 8 | *** | *** | *** |
| Compound 21 | *** | *** | *** |
| Compound 24 | *** | *** | *** |
| Compound 26 | *** | *** | *** |
| Compound 27 | *** | *** | *** |
| Compound 28 | *** | *** | *** |
| Compound 30 | *** | *** | *** |
| Compound 33 | *** | *** | *** |
| Compound 3 | *** | *** | *** |
| Compound 4 | *** | *** | *** |
| Compound 35 | *** | *** | *** |
| Compound 38 | *** | *** | *** |
| Compound 39 | *** | *** | *** |
| Compound 40 | *** | *** | *** |
| Compound 41 | *** | *** | *** |
| Compound 43 | *** | *** | *** |
| Compound 45 | *** | *** | *** |
| Compound 46 | *** | *** | *** |
| Compound 47 | *** | *** | *** |
| Compound 48 | *** | *** | *** |
| Compound 50 | *** | *** | *** |
| Compound 52 | *** | *** | *** |
| Compound 54 | *** | *** | *** |
| Compound 57 | *** | *** | *** |
| Compound 58 | *** | *** | *** |
| Compound 60 | *** | *** | *** |
| Compound 62 | *** | *** | *** |
| Compound 63 | *** | *** | *** |
| Compound 64 | *** | *** | *** |
| Compound 67 | *** | *** | *** |
| Compound 69 | *** | *** | *** |
| Compound 70 | *** | *** | *** |
| Compound 71 | *** | *** | *** |
| Compound 73 | *** | *** | *** |
| Compound 76 | *** | *** | *** |
| Compound 78 | *** | *** | *** |
| Compound 79 | *** | *** | *** |
| Compound 81 | *** | *** | *** |
| Compound 82 | *** | *** | *** |
| Compound 83 | *** | *** | *** |
| Compound 84 | *** | *** | *** |
| Compound 87 | *** | *** | *** |
| Compound 90 | *** | *** | *** |
| Compound 92 | *** | *** | *** |
| Compound 93 | *** | *** | *** |
| Compound 94 | *** | *** | *** |
| Compound 95 | *** | *** | *** |
| Compound 96 | *** | *** | *** |
| Compound 98 | *** | *** | *** |
| Compound 100 | *** | *** | *** |
| Compound 101 | *** | *** | *** |
| Compound 102 | *** | *** | *** |
| Compound 103 | *** | *** | *** |
| Compound 104 | *** | *** | *** |
| Compound 109 | *** | *** | *** |
| Compound 110 | *** | *** | *** |
| Compound 113 | *** | *** | *** |
| Compound 115 | *** | *** | *** |
| Compound 118 | *** | *** | *** |
| Compound 121 | *** | *** | *** |
| Compound 122 | *** | *** | *** |
| Compound 123 | *** | *** | *** |
| Compound 126 | *** | *** | *** |
| Compound 133 | *** | *** | *** |
| Compound 134 | *** | *** | *** |
| Compound 141 | *** | *** | *** |
| Compound 144 | *** | *** | *** |
| Compound 145 | *** | *** | *** |
| Compound 151 | *** | *** | *** |
| Compound 156 | *** | *** | *** |
| Compound 157 | *** | *** | *** |
| Compound 177 | *** | *** | *** |
| Compound 178 | *** | *** | *** |
| Compound 180 | *** | *** | *** |
| Compound 182 | *** | *** | *** |
| Compound 184 | *** | *** | *** |
| Compound 198 | *** | *** | *** |
| Compound 203 | *** | *** | *** |
| Compound 204 | *** | *** | *** |
| Compound 206 | *** | *** | *** |
| Compound 218 | *** | *** | *** |
| Compound 226 | *** | *** | *** |
| Compound 230 | *** | *** | *** |
| Compound 231 | *** | *** | *** |
| Compound 234 | *** | *** | *** |
| Compound 238 | *** | *** | *** |
| Compound 286 | *** | *** | *** |
| Compound 287 | *** | *** | *** |
| Compound 289 | *** | *** | *** |
| Compound 292 | *** | *** | *** |
| Compound 293 | *** | *** | *** |
| Compound 296 | *** | *** | *** |
| Compound 297 | *** | *** | *** |
| Compound 298 | *** | *** | *** |
| Compound 299 | *** | *** | *** |
| Compound 300 | *** | *** | *** |
| Compound 304 | *** | *** | *** |
| Compound 305 | *** | *** | *** |
| Compound 309 | *** | *** | *** |
| Compound 310 | *** | *** | *** |
| Compound 311 | *** | *** | *** |
| Compound 313 | *** | *** | *** |
| Compound 314 | *** | *** | *** |

In the table above for DC₅₀ and IP >=10 μM is *; <10 μM and >1 μM is **; <=1 μM is ***
For Emax >=90% is *; <90% and >50% is **; <=50% is ***

TABLE 4
A375.11 96 hours Viability_C4T

| | CTGlo | CTGlo |
|---|---|---|
| | A375.11 | A375.11 |
| | 96 hours | 96 hours |
| Compound | Emax | GI₅₀ nM |
| Compound 10 | *** | ** |
| Compound 100 | *** | *** |
| Compound 101 | *** | *** |
| Compound 102 | *** | *** |
| Compound 103 | ** | * |
| Compound 104 | ** | ** |
| Compound 106 | ** | ** |
| Compound 109 | *** | *** |
| Compound 11 | ** | * |
| Compound 110 | ** | * |
| Compound 113 | *** | *** |
| Compound 115 | *** | *** |
| Compound 116 | ** | * |
| Compound 117 | ** | *** |
| Compound 118 | ** | ** |
| Compound 119 | ** | ** |
| Compound 12 | *** | *** |
| Compound 121 | *** | *** |
| Compound 122 | *** | *** |
| Compound 123 | *** | *** |
| Compound 124 | ** | * |
| Compound 126 | ** | ** |
| Compound 128 | ** | * |
| Compound 129 | ** | ** |
| Compound 13 | ** | * |
| Compound 130 | ** | * |
| Compound 131 | ** | * |
| Compound 132 | ** | *** |
| Compound 133 | *** | ** |
| Compound 134 | ** | *** |
| Compound 136 | *** | *** |
| Compound 137 | ** | ** |
| Compound 138 | ** | ** |
| Compound 140 | *** | *** |
| Compound 141 | ** | *** |
| Compound 143 | *** | *** |
| Compound 144 | *** | *** |
| Compound 145 | ** | ** |
| Compound 146 | ** | * |
| Compound 147 | ** | * |
| Compound 149 | ** | * |
| Compound 15 | ** | * |
| Compound 151 | * | ** |
| Compound 153 | ** | * |
| Compound 155 | ** | * |
| Compound 156 | ** | *** |
| Compound 157 | ** | *** |
| Compound 158 | ** | *** |
| Compound 16 | *** | ** |
| Compound 160 | ** | *** |
| Compound 161 | ** | ** |
| Compound 163 | ** | ** |
| Compound 164 | ** | ** |
| Compound 165 | ** | * |
| Compound 166 | * | ** |
| Compound 167 | ** | ** |
| Compound 168 | ** | * |
| Compound 17 | ** | * |
| Compound 170 | ** | ** |
| Compound 172 | ** | ** |
| Compound 174 | ** | *** |
| Compound 176 | ** | *** |
| Compound 177 | ** | *** |
| Compound 178 | ** | ** |
| Compound 179 | ** | ** |
| Compound 180 | *** | *** |
| Compound 181 | *** | *** |
| Compound 182 | ** | ** |
| Compound 183 | ** | ** |
| Compound 184 | ** | ** |
| Compound 185 | ** | * |
| Compound 186 | * | ** |
| Compound 187 | ** | * |
| Compound 188 | ** | * |
| Compound 189 | *** | * |
| Compound 190 | *** | ** |
| Compound 191 | *** | *** |
| Compound 192 | *** | * |
| Compound 193 | ** | ** |
| Compound 194 | ** | * |
| Compound 195 | *** | * |
| Compound 196 | ** | *** |
| Compound 197 | ** | ** |
| Compound 198 | *** | ** |
| Compound 199 | ** | ** |
| Compound 200 | ** | ** |
| Compound 201 | ** | ** |
| Compound 203 | ** | * |
| Compound 204 | ** | *** |
| Compound 205 | ** | * |
| Compound 206 | ** | *** |
| Compound 208 | ** | *** |
| Compound 209 | *** | ** |
| Compound 21 | ** | ** |
| Compound 212 | ** | *** |
| Compound 214 | *** | *** |
| Compound 216 | ** | *** |
| Compound 217 | ** | ** |
| Compound 218 | ** | *** |
| Compound 219 | *** | * |
| Compound 22 | ** | * |
| Compound 220 | ** | * |
| Compound 221 | ** | ** |
| Compound 222 | ** | *** |
| Compound 223 | ** | ** |
| Compound 224 | ** | *** |
| Compound 225 | *** | *** |
| Compound 226 | ** | *** |
| Compound 227 | ** | ** |
| Compound 228 | ** | ** |
| Compound 229 | ** | *** |
| Compound 230 | ** | *** |
| Compound 231 | ** | ** |
| Compound 232 | ** | * |
| Compound 233 | ** | * |
| Compound 234 | ** | *** |
| Compound 235 | ** | * |
| Compound 236 | ** | ** |
| Compound 237 | ** | * |
| Compound 238 | ** | *** |
| Compound 24 | *** | *** |
| Compound 240 | ** | ** |
| Compound 241 | ** | ** |
| Compound 242 | ** | * |
| Compound 243 | ** | ** |
| Compound 245 | ** | *** |
| Compound 246 | *** | *** |
| Compound 247 | ** | ** |
| Compound 248 | ** | ** |
| Compound 249 | ** | *** |
| Compound 25 | ** | *** |
| Compound 250 | *** | *** |
| Compound 251 | ** | ** |
| Compound 252 | ** | * |
| Compound 253 | *** | ** |
| Compound 254 | *** | *** |
| Compound 255 | ** | * |
| Compound 256 | ** | *** |
| Compound 258 | ** | * |
| Compound 259 | *** | *** |
| Compound 26 | ** | *** |
| Compound 260 | ** | *** |
| Compound 261 | *** | *** |
| Compound 262 | ** | *** |
| Compound 263 | ** | * |
| Compound 264 | ** | ** |
| Compound 265 | *** | *** |
| Compound 266 | ** | * |
| Compound 267 | *** | *** |
| Compound 268 | *** | *** |
| Compound 269 | *** | *** |
| Compound 27 | *** | *** |
| Compound 270 | ** | *** |
| Compound 271 | *** | *** |
| Compound 272 | ** | * |
| Compound 273 | *** | *** |
| Compound 274 | *** | *** |
| Compound 275 | ** | * |
| Compound 276 | ** | ** |
| Compound 277 | ** | *** |
| Compound 278 | *** | *** |
| Compound 279 | ** | * |
| Compound 28 | *** | *** |
| Compound 280 | *** | *** |
| Compound 281 | *** | *** |
| Compound 282 | *** | *** |
| Compound 283 | ** | *** |
| Compound 284 | *** | *** |
| Compound 285 | *** | *** |
| Compound 286 | *** | *** |
| Compound 287 | *** | *** |
| Compound 288 | ** | * |
| Compound 289 | *** | *** |
| Compound 290 | ** | * |
| Compound 291 | * | ** |
| Compound 292 | ** | *** |
| Compound 293 | ** | *** |
| Compound 294 | ** | ** |
| Compound 295 | * | ** |
| Compound 296 | ** | *** |
| Compound 297 | ** | ** |
| Compound 298 | *** | *** |
| Compound 299 | ** | ** |
| Compound 3 | *** | *** |
| Compound 30 | *** | ** |
| Compound 300 | ** | *** |
| Compound 301 | ** | *** |
| Compound 302 | ** | *** |
| Compound 303 | ** | * |
| Compound 304 | ** | ** |
| Compound 305 | ** | *** |
| Compound 306 | ** | * |
| Compound 307 | ** | ** |
| Compound 309 | ** | * |
| Compound 31 | *** | ** |
| Compound 310 | ** | ** |
| Compound 311 | ** | ** |
| Compound 313 | ** | *** |
| Compound 314 | ** | *** |
| Compound 32 | *** | ** |
| Compound 321 | ** | * |
| Compound 322 | ** | * |
| Compound 33 | *** | *** |
| Compound 35 | ** | ** |
| Compound 38 | *** | *** |
| Compound 39 | *** | *** |
| Compound 4 | *** | *** |
| Compound 40 | ** | ** |
| Compound 41 | *** | *** |
| Compound 43 | ** | *** |
| Compound 45 | *** | *** |
| Compound 46 | *** | *** |
| Compound 47 | ** | ** |
| Compound 48 | *** | *** |
| Compound 50 | *** | *** |
| Compound 51 | *** | ** |
| Compound 52 | ** | * |
| Compound 53 | ** | ** |
| Compound 54 | *** | *** |
| Compound 56 | ** | * |
| Compound 57 | ** | ** |
| Compound 58 | *** | *** |
| Compound 59 | ** | ** |
| Compound 6 | *** | *** |
| Compound 60 | *** | *** |
| Compound 61 | ** | * |
| Compound 62 | *** | *** |
| Compound 63 | *** | *** |
| Compound 64 | ** | ** |
| Compound 65 | ** | * |
| Compound 67 | *** | *** |
| Compound 68 | *** | * |
| Compound 69 | *** | *** |
| Compound 7 | *** | *** |
| Compound 70 | *** | *** |
| Compound 71 | *** | *** |
| Compound 73 | *** | ** |
| Compound 76 | *** | *** |
| Compound 78 | *** | *** |
| Compound 79 | ** | ** |
| Compound 8 | *** | *** |
| Compound 80 | ** | * |
| Compound 81 | ** | ** |
| Compound 82 | ** | ** |
| Compound 83 | *** | *** |
| Compound 84 | ** | ** |
| Compound 85 | *** | *** |
| Compound 86 | *** | *** |
| Compound 87 | *** | *** |
| Compound 9 | *** | *** |
| Compound 90 | ** | *** |
| Compound 92 | *** | *** |
| Compound 93 | *** | *** |
| Compound 94 | *** | *** |
| Compound 95 | ** | *** |
| Compound 96 | *** | *** |
| Compound 96 | ** | *** |

In the table above for GI₅₀ >=10 μM is *; <10 μM and >1 μM is **; <=1 μM is ***
For Emax >=90% is *; <90% and >50% is **; <=50% is ***

### Example 6 In vivo PKPD study and efficacy study of Compound 83, Compound 93, Compound 70, and Compound 54

### Method

For the pharmacokinetic/pharmacodynamic (PKPD) study, compounds were tested in the A375 human melanoma xenografts. Known BRAF inhibitors encorafenib, and dabrafenib were used as positive controls. Encorafenib, dabrafenib, Compound 83, Compound 93, Compound 70, and Compound 54 were administered to BALB/c nude mice bearing A375 tumors on PG-D1 and dosed orally (PO) once. Encorafenib was dosed at 35 mg/kg. Dabrafenib was dosed at 100 mg/kg. Compound 83, Compound 93, Compound 70, and Compound 54 were dosed at 10 mg/kg. The plasma and tumor samples were collected at 1, 6, 12 and 24 hours, and liver samples were collected at 6 hours and 12 hours post the single dose. This study was non-GLP study.

For efficacy study, compounds were tested in the A375 human melanoma xenografts. Known BRAF inhibitors encorafenib, and dabrafenib were used as positive controls. Encorafenib and dabrafenib were administered to BALB/c nude mice bearing A375 tumors on PG-D1 and dosed PO daily (QD) for 3 weeks. Encorafenib was dosed at 35 mg/kg. Dabrafenib was dosed at 100 mg/kg. Compound 83, Compound 93, Compound 70, and Compound 54 were administered to BALB/c nude mice bearing A375 tumors on PG-DO and dosed PO twice per day (BID) for 3 weeks. Compound 83, Compound 93, Compound 70, and Compound 54 were dosed at 10 mg/kg.

### Experimental Design

TABLE 5
Study Design for PKPD Study

| | | | | | | Time point for | |
|---|---|---|---|---|---|---|---|
| | | | Dose | Dosing | | Plasma and | Time point |
| Group | N^{a} | Treatment^{b} | (mg/kg) | Route | Schedule | Tumor^{c} | for Liver^{c} |
| 8 | 3 | Vehicle | — | p.o. | Single | 6 h | — |
| | | | | | dose | (Only Tumor) | |
| 9 | 3 | Encorafenib | 35 | p.o. | Single | 1 h | — |
| | | | | | dose | | |
| | 3 | Encorafenib | 35 | p.o. | Single | 6 h | 6 h |
| | | | | | dose | | |
| | 3 | Encorafenib | 35 | p.o. | Single | 12 h | 12 h |
| | | | | | dose | | |
| | 3 | Encorafenib | 35 | p.o. | Single | 24 h | — |
| | | | | | dose | | |
| 10 | 3 | Dabrafenib | 100 | p.o. | Single | 1 h | — |
| | | | | | dose | | |
| | 3 | Dabrafenib | 100 | p.o. | Single | 6 h | 6 h |
| | | | | | dose | | |
| | 3 | Dabrafenib | 100 | p.o. | Single | 12 h | 12 h |
| | | | | | dose | | |
| | 3 | Dabrafenib | 100 | p.o. | Single | 24 h | — |
| | | | | | dose | | |
| 11 | 3 | Compound | 10 | p.o. | Single | 1 h | — |
| | | 83 | | | dose | | |
| | 3 | Compound | 10 | p.o. | Single | 6 h | 6 h |
| | | 83 | | | dose | | |
| | 3 | Compound | 10 | p.o. | Single | 12 h | 12 h |
| | | 83 | | | dose | | |
| | 3 | Compound | 10 | p.o. | Single | 24 h | — |
| | | 83 | | | dose | | |
| 12 | 3 | Compound | 10 | p.o. | Single | 1 h | — |
| | | 93 | | | dose | | |
| | 3 | Compound | 10 | p.o. | Single | 6 h | 6 h |
| | | 93 | | | dose | | |
| | 3 | Compound | 10 | p.o. | Single | 12 h | 12 h |
| | | 93 | | | dose | | |
| | 3 | Compound | 10 | p.o. | Single | 24 h | — |
| | | 93 | | | dose | | |
| 13 | 3 | Compound | 10 | p.o. | Single | 1 h | — |
| | | 70 | | | dose | | |
| | 3 | Compound | 10 | p.o. | Single | 6 h | 6 h |
| | | 70 | | | dose | | |
| | 3 | Compound | 10 | p.o. | Single | 12 h | 12 h |
| | | 70 | | | dose | | |
| | 3 | Compound | 10 | p.o. | Single | 24 h | — |
| | | 70 | | | dose | | |
| 14 | 3 | Compound | 10 | p.o. | Single | 1 h | — |
| | | 54 | | | dose | | |
| | 3 | Compound | 10 | p.o. | Single | 6 h | 6 h |
| | | 54 | | | dose | | |
| | 3 | Compound | 10 | p.o. | Single | 12 h | 12 h |
| | | 54 | | | dose | | |
| | 3 | Compound | 10 | p.o. | Single | 24 h | — |
| | | 54 | | | dose | | |

Note:
^{a}N: animal number. Dosing volume: adjusted dosing volume based on body weight 10 μL/g.
^{b}The formulation for vehicle and Compound 83, Compound 93, Compound 70, and Compound 54 was 20% PEG 400 + 80% (25% SBE-β-CD in ddH₂O). The formulation for encorafenib was 0.5% CMC-Na + 0.5% Tween-80 in ddH₂O. The formulation for dabrafenib was 10% PEG400 + 90% (11.1% HP-β-CD in ddH₂O).
^{c}Plasma and liver samples were collected at predetermined time for PK analysis. Tumor samples were collected at predetermined time points for PK and Western blot (WB) analysis (BRAF and p-ERK).

TABLE 6
Study Design for the Efficacy Study

| | | | Weeks 1-3 | | | |
|---|---|---|---|---|---|---|
| | | | Dose | Dosing | | Time |
| Group | N^{a} | Treatment^{b} | (mg/kg) | Route | Schedule | point^{c} |
| 1 | 6 | Vehicle | — | p.o. | BID × 21 days | — |
| 2 | 6 | Encorafenib | 35 | p.o. | QD × 21 days | 6 h |
| 3 | 6 | Dabrafenib | 100 | p.o. | QD × 21 days | 6 h |
| 4 | 6 | Compound | 10 | p.o. | BID × 21 days | 6 h |
| | | 83 | | | | |
| 5 | 6 | Compound | 10 | p.o. | BID × 21 days | 6 h |
| | | 93 | | | | |
| 6 | 6 | Compound | 10 | p.o. | BID × 21 days | 6 h |
| | | 70 | | | | |
| 7 | 6 | Compound | 10 | p.o. | BID × 21 days | 6 h |
| | | 54 | | | | |

Note:
^{a}N: animal number. Dosing volume: adjusted dosing volume based on body weight 10 μL/g.
^{b}The formulation for vehicle and compound 83, compound 93, compound 70, compound 54 was 20% PEG400 + 80% (25% SBE-β-CD in ddH₂O). The formulation for encorafenib was 0.5% CMC-Na + 0.5% Tween-80 in ddH₂O. The formulation for dabrafenib was 10% PEG400 + 90% (11.1% HP-β-CD in ddH₂O).
^{c}All mice were euthanized on PG-D 21, the plasma and liver samples were collected at 6 h post last dose for PK analysis.

### Materials

### Animals and Housing Condition

One hundred and seventeen female BALB/c nude mice procured from Shanghai Lingchang Biological Science and Technology Co., LTD between the ages of six to eight weeks and with body weight between eighteen and twenty-two grams were used for the study. A spare mouse was also procured.

The mice were kept in individual ventilation cages at constant temperature and humidity with 3 or 6 animals in each cage. The cage was kept between approximately 20 to 26° C. with a humidity of 40-70%. The cages were made of polycarbonate with a size of 375 mm×215 mm×180 mm and corn cob bedding which was changed twice per week.

The animals were fed irradiation sterilized dry granule food during the entire study period. The animals had free access to food and sterile drinking water. Cages were identified with the following information: number of animals, sex, strain, date received, treatment, study number, group number and the starting date of the treatment and each animal was marked by ear coding.

Test Articles

| Compound | Molecular Weight | Formula Weight |
|---|---|---|
| Encorafenib | 540.01 | 540.01 |
| Dabrafenib | 519.56 | 519.56 |
| Compound 83 | 900.98 | 900.98 |
| Compound 93 | 894.01 | 894.01 |
| Compound 70 | 870.93 | 870.93 |
| Compound 54 | 882.99 | 882.99 |

Reagents and Materials for PD
Reagents and Consumables

| No. | Reagents/Consumable | Vendor | Catalog # |
|---|---|---|---|
| 1 | RIPA Buffer | Sigma | R0278 |
| 2 | Phosphatase Inhibitor Cocktail 2 | Sigma | P5726 |
| 3 | Protease Inhibitor Cocktail | Roche | 04693124001 |
| 4 | Pierce ™ BCA Protein Assay Kit | Thermo | 23225 |
| | | Scientific | |
| 5 | NuPAGE ™ LDS Sample Buffer (4×) | Invitrogen | NP0007 |
| 6 | NuPAGE ™ Sample Reducing Agent (10×) | Invitrogen | NP0009 |
| 7 | PageRuler ™ Prestained Protein Ladder | Thermo | 26616 |
| | | Scientific | |
| 8 | NuPAGE ™ Novex 4-12% Bis-Tris Gel 1.0 mm, 26 Well | Invitrogen | WG1403BOX |
| 9 | NuPAGE ™ MES SDS Running Buffer (20×) | Invitrogen | NP0002 |
| 10 | iBlot ™ 2 Transfer Stack Regular | Invitrogen | IB23001 |
| 11 | Restore ™ Western Blot Stripping Buffer | Thermo | 21059 |
| | | Scientific | |
| 12 | 20 × TBS | Bio-Serve | BS-P-15 |
| 13 | Tween 20 | Sigma | P2287 |
| 14 | SuperSignal ™ West Femto Maximum Sensitivity Substrate | Thermo | 34096 |
| | | Scientific | |
| 15 | Bovine Serum Albumin | BBI life | A600332 |
| | | sciences | |
| | | & services | |
| 16 | Nonfat Milk | BD Difco | 232100 |

1 × TBST: added 50 mL 20 × TBS to 949 mL H₂O, and then added 1 mL Tween 20, mixed well.
Running buffer: added 50 mL 20 × MES SDS Running Buffer to 950 mL H₂O, mixed well.

Instruments

| No. | Instrument | Vendor | Model # |
|---|---|---|---|
| 1 | Tissuelyser | Shanghai Jingxin | JXFSTPRP-CL |
| 2 | Electro- thermal incubator | Boxun | HPX-9162 MBE |
| 3 | SpectraMax 340PC | Molecular Devices | 340PC |
| 4 | Digital Dry Baths | Qiling | GL-150B |
| 5 | XCell4 SureLock ® Midi-cell | Thermo Scientific | EI0001 |
| 6 | PowerPac ™ Basic Power Supply | Bio-Rad | 1645050 |
| 7 | iBlot ®2 Gel Transfer Device | Invitrogen | iBlot2 |
| 8 | Novex ™ Power Supply Adapters | Invitrogen | ZA10001 |
| 9 | Eppendorf Centrifuge | Eppendorf | 5424R |
| 10 | Amersham Imager 680 | GE Healthcare | AI680QC |
| 11 | Orbital Shaker | Qiling | TS-1 |
| 12 | Beads | Qiagen | 69989 |

| No. | Antibodies | Vendor | Catalog # |
|---|---|---|---|
| 1 | B-Raf (D9T6S) Rabbit mAb | Cell Signaling | CST-14814 |
| | | Technology | |
| 2 | Phospho-p44/42 MAPK (Erk1/2) | Cell Signaling | CST-9101 |
| | (Thr202/Tyr204) Antibody | Technology | |
| 3 | GAPDH (D16H11) XP ® Rabbit mAb | Cell Signaling | CST-5174 |
| | | Technology | |
| 4 | Goat Anti-Rabbit IgG H&L (HRP) | Abcam | ab6721 |
| 5 | Goat Anti-Mouse IgG H&L (HRP) | Abcam | ab97023 |

### Experimental Procedures

### Cell Culture

The A375 tumor cells were maintained in vitro in DMEM medium supplemented with 100 fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation. The details of reagents are shown in Table 7.

TABLE 7
Vendor and Cat No. of Cell Line and Reagents

| Cell Line/Reagent | Vendor | Catalog # |
|---|---|---|
| A375 | ATCC | CRL-1619 |
| DMEM | Gibco | 11995-065 |
| Penicillin-Streptomycin | Gibco | 15240-062 |
| 0.25% Trypsin-EDTA | Gibco | 25200-072 |
| FBS | Corning | 35-081-CV |
| DPBS | Corning | 21-031-CVC |
| Matrigel | Corning | 354234 |

### Tumor Inoculation

Each mouse was inoculated subcutaneously at the right flank with A375 tumor cells (5×10⁶) in 0.2 mL of PBS with Matrigel (PBS: Matrigel=1:1) supplemented for tumor development. Animals were grouped for the treatment on day 16 after tumor inoculation when the average tumor volume reached 221 mm³ for efficacy study and 386 mm³ for PKPD study. The test articles administration and the animal numbers in each group are shown in the above experimental Tables.

### Vehicle Preparation

### Preparation of 0.5% CMC-Na+0.5% Tween-80 in ddH₂O

1.500 g of CMC-Na was accurately weighed into a 500 mL glass bottle. Then 200 mL ddH₂O was added and the mixture was stirred for a while. 1.5 mL Tween-80 was then added to the mixture and stirring continued followed by addition of ddH₂O to a total volume of 300 mL. The mixture was mixed well and allowed to stir overnight until a clear solution formed.

### Preparation of 11.1% HP-β-CD in ddH₂O

22.2 g HP-β-CD was accurately weighed into a 200 mL glass bottle. 160 mL ddH₂O was then added and the mixture was stirred to obtain a clear solution. Then ddH₂O was added to a total volume of 200 mL and the mixture was mixed well.

### Preparation of 25% SBE-β-CD in ddH₂O

100 g of SBE-β-CD was accurately weighed into a 1 μL glass bottle. 300 mL volume of ddH₂O was then added and the mixture was stirred to obtain a clear solution. Then ddH₂O was added to a total volume of 400 mL and the mixture was mixed well.

### Testing Article Formulation Preparation

TABLE 8
Formulation of C4T's Preparation

| | | | | Volume of | Volume of 25% | |
|---|---|---|---|---|---|---|
| | | Conc. of | Compound | PEG400 to be | SBE-β-CD to be | Dosing |
| Compound | Dose | solution | amount | added (mL) | added (mL) | Solution |
| ID ^{a} | (mg/kg) | (mg/mL) | (mg) | (20%) | (80%) | observations |
| Compound | 10 | 1 | 13.56 | 2.705 | 10.819 | Clear |
| 83 | | | | | | Solution |
| Compound | 10 | 1 | 13.65 | 2.682 | 10.729 | Clear |
| 54 | | | | | | Solution |
| Compound | 10 | 1 | 14.06 | 2.718 | 10.872 | Clear |
| 70 | | | | | | Solution |

Note:
^{a} The formulations were prepared twice per week.

TABLE 9
Formulation of Compound 93 Preparation

| | | | | Volume of | Volume of | Volume | | |
|---|---|---|---|---|---|---|---|---|
| | | | | PEG400 to | 25% SBE-β- | of 6N | Final pH | |
| | | Conc. of | | be added | CD to be | HCl to be | (adjust | Dosing |
| Compound | Dose | solution | Amount | (mL) | added (mL) | added | with 6N | Solution |
| ID ^{a} | (mg/kg) | (mg/mL) | (mg) | (20%) | (80%) | (mL) | HCl) | observations |
| Compound | 10 | 1 | 13.55 | 2.684 | 10.728 | 0.007 | 3.03 | Clear |
| 93 | | | | | | | | Solution |

Note:
^{a} The formulation was prepared twice per week.

TABLE 10
Formulation of Encorafenib Preparation

| | | | | Volume of 0.5% CMC- | |
|---|---|---|---|---|---|
| | | Conc. of | Compound | Na + 0.5% Tween-80 in | Dosing |
| Compound | Dose | solution | amount | ddH₂O to be added | solution |
| ID ^{b} | (mg/kg) | (mg/mL) | (mg) | (mL) | observations |
| Encorafenib | 35 | 3.5 | 23.39 | 6.658 | Suspension |

Note:
a. The formulation was prepared twice per week.

TABLE 11
Formulation of Dabrafenib Preparation

| | | | | Volume of | Volume of 11.1% | |
|---|---|---|---|---|---|---|
| | | Conc. of | Compound | PEG400 to be | HP-β-CD to be | Dosing |
| Compound | Dose | solution | amount | added (mL) | added (mL) | solution |
| ID ^{b} | (mg/kg) | (mg/mL) | (mg) | (10%) | (90%) | observations |
| Dabrafenib | 100 | 10 | 67.73 | 0.677 | 6.090 | Suspension |

Note:
a. The formulation was prepared twice a week.

### Observations

At the time of routine monitoring, the animals were checked daily for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset. Animals that were observed to be in a continuing deteriorating condition or their tumor size exceeding 3,000 mm³ were euthanized prior to death or before reaching a comatose state.

### Tumor Measurements and the Endpoints

Prior to the onset of drug treatment, mice were measured for tumor size in two dimensions using a caliper, and the tumor volume (mm³) was calculated using formula V=0.5 a×b² where a and b are the long and short diameters of the tumor in mm, respectively. Mice were randomized into different treatment groups based on the tumor volume.

The major endpoint was to see if the tumor growth could be delayed or mice could be cured. The tumor size was then used for calculations of T/C value. The T/C value (in percent) is an indication of antitumor effectiveness; T and C are the mean volumes of the treated and control groups, respectively on specified day.

TGI was calculated for each group using the formula: TGI (%)=[1−(Tᵢ−T₀)/(Cᵢ−C₀)]×100; Tᵢ is the average tumor volume of a treatment group on a given day, T₀ is the average tumor volume of the treatment group on the day of treatment start, Cᵢ is the average tumor volume of the vehicle control group on the same day with Tᵢ, and C₀ is the average tumor volume of the vehicle group on the day of treatment start.

### Sample Collection

### Plasma Sample Preparation

The plasma samples were collected at predetermined time points for PK analysis. The mice were euthanized at predetermined time points post the treatment. The blood samples were collected into the 1.5 mL Eppendorf tubes containing 15 μL of 0.5 M EDTA-K2. And then the anticoagulant blood samples were centrifuged at 2,500 g at 4° C. for 15 minutes. The plasma samples were separated into 2 aliquots. The 1^{st} was used for PK analysis, and the 2^{nd} served as backup. The samples were stored at −80° C. before bioanalysis.

### Tumor Sample Preparation

The tumor samples were collected at predetermined time points for PK and WB analysis (B-RAF and p-ERK). The tumor was dissected out and split into 3 parts. The 1^{st} part was used for PK analysis, the 2^{nd} part was used for WB analysis and the last one served as backup for study. The samples were transferred into pre-labeled tubes and quickly froze in liquid nitrogen within 2 minutes after dissection. The samples were stored at −80° C. before bioanalysis.

### Liver Sample Preparation

Liver samples were collected at predetermined time points for PK analysis. The left lobe of the liver was dissected out and split into 2 parts. The 1^{st} part was used for PK analysis and the 2^{nd} part served as the backup for the study. The samples were transferred into pre-labeled tubes and quickly froze in liquid nitrogen within 2 minutes after dissection. The samples were stored at −80° C. before bioanalysis.

### Pharmacokinetics Assay

### Pharmacokinetics Assay for efficacy study

For efficacy study, the harvested plasma and liver were processed for pharmacokinetics analysis by LC/MS/MS with a concentration range of 2.00-2000 ng/mL for Encorafenib and Dabrafenib in plasma, 0.20-200 ng/mL for Encorafenib and Dabrafenib in liver, 50.0-50000 ng/mL for compound 83 and compound 70 in plasma, 20.0-20000 ng/mL for Compound 93 and Compound 54 in plasma, 5.00-5000 ng/mL for compound 93, compound 83, compound 70 and compound 54 in liver.

### Pharmacokinetics Assay for PKPD Study

For PKPD study, the harvested plasma, tumor and liver were processed for pharmacokinetics analysis by LC/MS/MS with a concentration range of 0.5-1000 ng/mL for Encorafenib and Dabrafenib in plasma, 0.05-100 ng/mL for Encorafenib and Dabrafenib in tumor, 0.2-200 ng/mL for Encorafenib and Dabrafenib in liver, 2.0-2000 ng/mL for compound 93, compound 83, compound 54 and compound 70 in liver, 50.0-50000 ng/mL for compound 83 and compound 70 in plasma, 0.5-1000 ng/mL for compound 83 and compound 70 in tumor, 1.0-2000 ng/mL for compound 93 in plasma, 10.0-20000 ng/mL for compound 54 in plasma, 0.5-500 ng/mL for compound 93 and compound 54 in tumor.

### Western Blotting Assay

### Protein Extraction & Protein Quantification

The previously snap-frozen cells were removed from the −80° C. freezer and then cut up to about 30-100 mg of tissue per sample. The tissues were then placed in 2 mL microcentrifuge tube and 400 μL of RIPA Buffer was then added. The RIPA Buffer contained 1% Protease Inhibitor Cocktail and 1% Phosphatase Inhibitor Cocktail 2.

The tumors were then ground with a freeze grinder at 50 Hz for five minutes and then the tissue lysates were kept on ice for thirty minutes. The lysates were centrifuged at 15,000 rpm at 4° C. for ten minutes and then the supernatants were gently transferred to a new microcentrifuge tube kept on ice.

The protein concentration was measured with a Pierce™ BCA Protein Assay Kit. Then the samples were diluted, according to the result of BCA protein quantification, to the same final concentration (at 2 μg/μL) using RIPA buffer plus 4× LDS Sample Buffer and 10× Sample Reducing Agent. The samples were then heated at 100° C. for ten minutes. The denatured samples were then used for Western blotting or kept in the −80° C. refrigerator for long term storage.

### Western Blotting

The protein samples were loaded to NuPAGE® Novex 4-12% Bis-Tris gel with 10 μL per slot. The electrophoresis with MES running buffer was ran at 80 V for 30 minutes and then at 120 V for 90 minutes. Then protein was transferred to NC (nitrocellulose) membranes with iBlot®2 Gel Transfer Device, P3 for 7 minutes, or with Mini Trans-Blot® Electrophoretic Transfer Cell device (in pre-cooled 1× wet transfer buffer) in an ice bath at 100 V for 90 minutes. Afterwards, the membranes were cut, the interest membranes parts were kept and washed with 5-10 mL 1× TBST three times for 5 minutes each. Then the non-specific protein was blocked by 5-10 mL 1× TBST with 5% nonfat milk at room temperature for 1 hour. The membrane was then washed with 5-10 mL 1× TBST three times for 5 minutes each. The membrane was then incubated with 5-10 mL diluted primary antibody in 1× TBST with 5% BSA at 4° C. with gentle agitation overnight. Next, the membrane was washed with 5-10 mL 1× TBST three times for 10 minutes each. The resulting membrane was incubated with 5-10 mL HRP-conjugated secondary antibody (generally 1:3000) with gentle agitation for 1 hour at room temperature. Then the membrane was washed with 5-10 mL 1× TBST three times for 10 minutes each. Afterwards, the HRP substrate from West Femto Maximum Sensitivity kit was added to the NC membranes and chemiluminescence was detected by AI680QC.

### Quantification of Band Intensity

The intensity of individual bands was quantified using 1D quant (GE) densitometry software.

### Statistical Analysis

Band intensity of individual bands was obtained for analysis. Protein expression was quantitated in relation to a reference protein, and then the normalized folds of protein expression in each sample were ratios to vehicle group.

The normalized data were interpreted by GraphPad Prism. Statistical analysis of difference in the protein expression of interests among the groups was conducted.

### Data Management and Statistical Analyses

A one-way ANOVA followed by Dunnett's multiple comparison test was performed to compare tumor volume among vehicle group and treatment groups. All data were analyzed using GraphPad Prism, ns indicates p>0.5, ** indicates p<0.01, *** indicates p0.001 and indicates p<0.0001.

### Results

### Results for Efficacy Study

### Body Weight Change

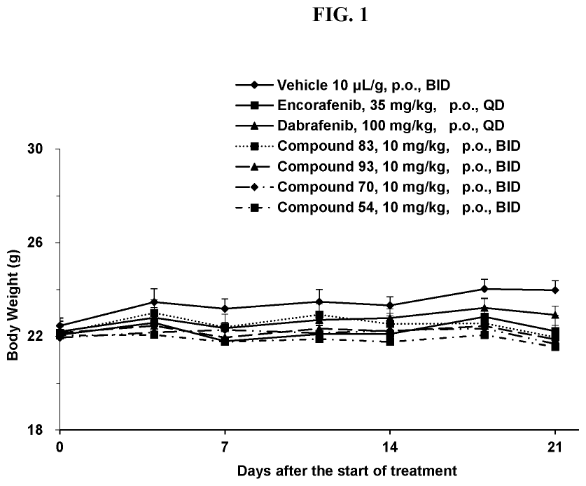

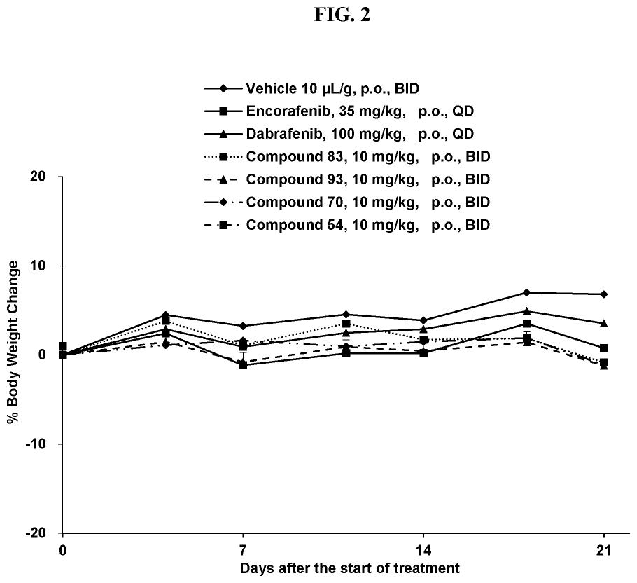

Animal body weight was monitored regularly as an indirect measurement of toxicity. Body weight changes after administration of the test articles are shown in FIG. 1 and FIG. 2.

### Tumor Growth Curve

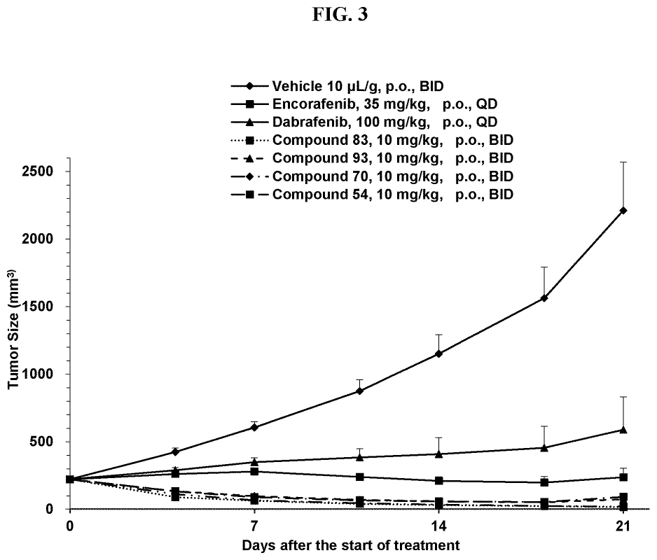

Tumor growth curves are shown in FIG. 3.

### Tumor Volume Trace

Mean tumor size over time in female BALB/c nude mice bearing A375 melanoma cancer xenografts is shown in Table 12.

TABLE 12
The Tumor Volume Trace Over Time (mm³)

| | Days | | | | | | |
|---|---|---|---|---|---|---|---|
| Group # | 0^{a} | 4 | 7 | 11 | 14 | 18 | 21 |
| Vehicle 10 μL/g, | 221 ± 10 | 423 ± 29 | 605 ± 41 | 874 ± 83 | 1150 ± 140 | 1562 ± 228 | 2211 ± 358 |
| BID × 21 days | | | | | | | |
| Encorafenib, 35 mg/kg, | 221 ± 10 | 260 ± 22 | 278 ± 17 | 238 ± 18 | 209 ± 31 | 196 ± 47 | 235 ± 68 |
| QD × 21 days | | | | | | | |
| Dabrafenib, 100 mg/kg, | 221 ± 10 | 288 ± 19 | 348 ± 33 | 383 ± 64 | 407 ± 122 | 454 ± 159 | 588 ± 244 |
| QD × 21 days | | | | | | | |
| Compound 83, 10 mg/kg, | 221 ± 11 | 89 ± 11 | 62 ± 7 | 39 ± 3 | 31 ± 2 | 22 ± 1 | 15 ± 1 |
| BID × 21 days | | | | | | | |
| Compound 93, 10 mg/kg, | 221 ± 9 | 133 ± 6 | 97 ± 6 | 69 ± 5 | 59 ± 5 | 52 ± 4 | 73 ± 18 |
| BID × 21 days | | | | | | | |
| Compound 70, 10 mg/kg, | 221 ± 10 | 111 ± 13 | 66 ± 6 | 44 ± 3 | 34 ± 2 | 24 ± 2 | 19 ± 2 |
| BID × 21 days | | | | | | | |
| Compound 54, 10 mg/kg, | 221 ± 11 | 132 ± 13 | 90 ± 14 | 65 ± 13 | 57 ± 13 | 53 ± 13 | 90 ± 27 |
| BID × 21 days | | | | | | | |

Note:
^{a}Data are shown as Mean ± SEM.

### Tumor Growth Inhibition Analysis

Tumor growth inhibition of test articles in the treatment of female BALB/c nude mice bearing A375 xenografts was calculated based on tumor volume measured on PG-D21. TGI and T/C were calculated respectively, and the data are detailed in Table 13.

TABLE 13
Tumor Growth Inhibition Analysis Compared (T/C and TGI)

| | Tumor Size | | | |
|---|---|---|---|---|
| | (mm³)^{a} on | | | |
| | PG-D21 | T/C^{b} | | |
| Treatment | on PG-D21 | (%) | TGI^{c} (%) | P value^{d} |
| Vehicle, 10 μL/g, BID × 21 days | 2211 ± 358 | — | — | — |
| Encorafenib, 35 mg/kg, QD × 21 days | 235 ± 68 | 10.6 | 99.3 | **** |
| Dabrafenib, 100 mg/kg, QD × 21 days | 588 ± 244 | 26.6 | 81.5 | **** |
| Compound 83, 10 mg/kg, BID × 21 | 15 ± 1 | 0.7 | 110.3 | **** |
| days | | | | |
| Compound 93, 10 mg/kg, BID × 21 | 73 ± 18 | 3.3 | 107.4 | **** |
| days | | | | |
| Compound 70, 10 mg/kg, BID × 21 | 19 ± 2 | 0.9 | 110.1 | **** |
| days | | | | |
| Compound 54, 10 mg/kg, BID × 21 | 90 ± 27 | 4.1 | 106.6 | **** |
| days | | | | |

^{a}Data are shown as Mean ± SEM.
^{b}Antitumor activity (T/C) was determined by dividing the average tumor volume for treated group (T) by the average tumor volume for control group (C).
^{c}Tumor Growth Inhibition (TGI) was calculated using the formula: TGI (%) = (1 − (T₂₁ − T₀)/(C₂₁ − C₀))*100, where T21 = mean tumor size at the terminal end of treatment group at day 21, T₀ = mean tumor size at day 0 of treatment group; C₂₁ = mean tumor size at the terminal end of control group at day 21; and C₀ = mean tumor size at day 0 of control group.
^{d}A one-way ANOVA followed by Dunnett's multiple comparison test was performed to compare tumor volume among vehicle groups and treatment groups, **** indicates p < 0.0001.

### Pharmacokinetics Results

The concentration of test articles in plasma and liver samples collected from the treatment of female BALB/c nude mice bearing A375 tumor are shown in Table 14 and FIG. 4.

TABLE 14
Mean Concentration of Test Articles in Plasma

| | | Concentration | Concentration |
|---|---|---|---|
| | Sample | in Plasma | in Liver |
| Group | Time | (ng/mL) ± SEM | (ng/g) ± SEM |
| Encorafenib, 35 mg/kg, | 6 h | 992 ± 559 | 481 ± 272 |
| p.o., QD × 21 days | | | |
| Dabrafenib, 100 mg/kg, | 6 h | 86.1 ± 15.4 | 216 ± 30.8 |
| p.o., QD × 21 days | | | |
| Compound 83, 10 mg/kg, | 6 h | 17067 ± 1507 | 11000 ± 1051 |
| p.o., BID × 21 days | | | |
| Compound 93, 10 mg/kg, | 6 h | 1098 ± 389 | 652 ± 180 |
| p.o., BID × 21 days | | | |
| Compound 70, 10 mg/kg, | 6 h | 16083 ± 1345 | 6392 ± 570 |
| p.o., BID × 21 days | | | |
| Compound 54, 10 mg/kg, | 6 h | 3987 ± 634 | 4243 ± 955 |
| p.o., BID × 21 days | | | |

### Results for PKPD Study

### Pharmacokinetics Results

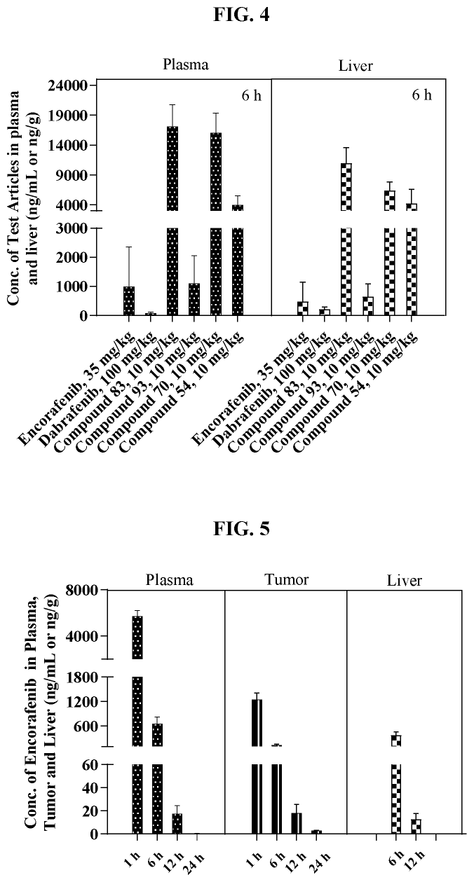

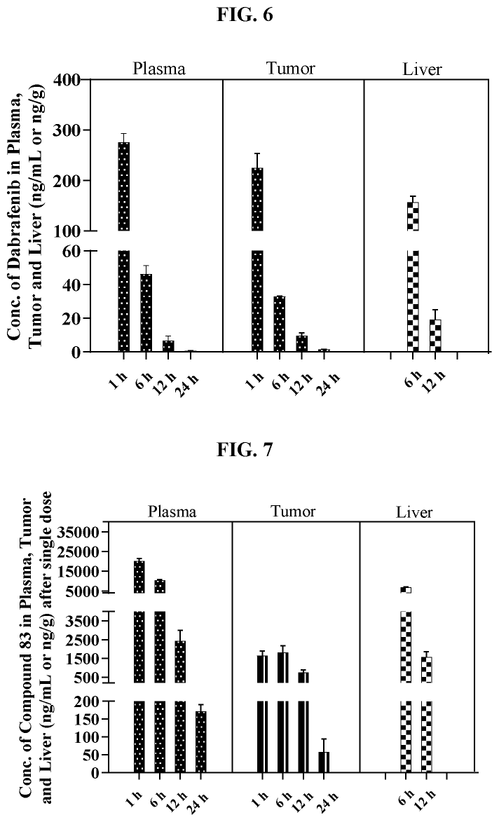

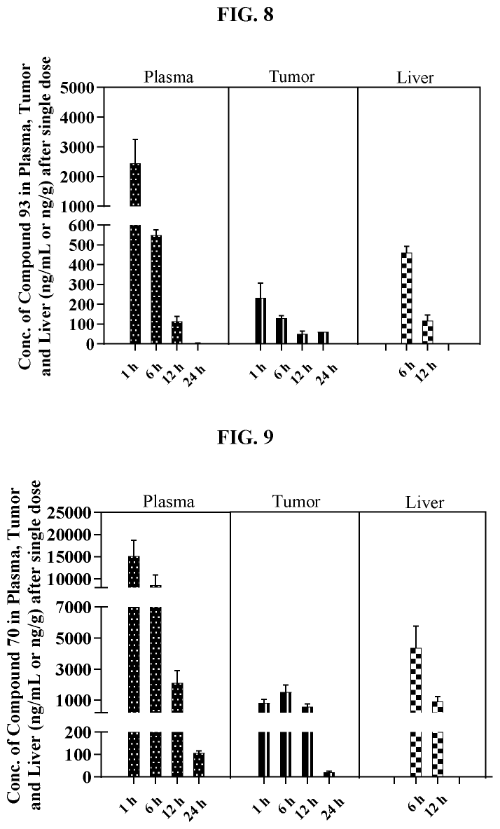

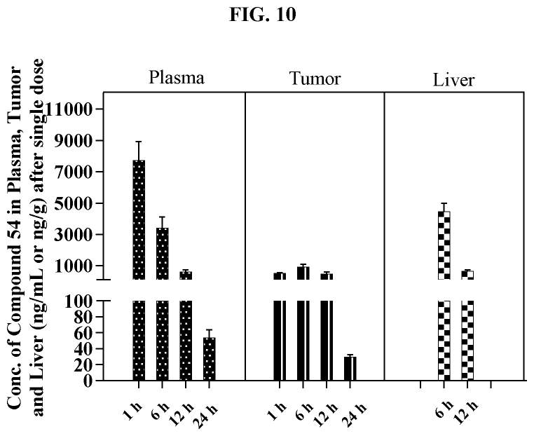

The concentration of test articles in plasma, tumor and liver collected from the treatment of female BALB/c nude mice bearing A375 tumor are shown in Table 15, FIG. 5-10.

TABLE 15
Mean Concentration of Test Articles in Plasma

| | | | Concen- | Concen- | Concen- |
|---|---|---|---|---|---|
| | | | tration | tration | tration |
| | | | in Plasma | in Tumor | in Liver |
| | Sample | | (ng/mL) ± | (ng/g) ± | (ng/g) ± |
| Group | Time | | SEM | SEM | SEM |
| Encorafenib, | 1 | h | 5723 ± 493 | 1250 ± 161 | — |
| 35 mg/kg, | 6 | h | 654 ± 169 | 135 ± 22.8 | 375 ± 82.0 |
| single dose | 12 | h | 17.3 ± 7.09 | 18.1 ± 7.47 | 12.6 ± 5.14 |
| | 24 | h | 0.895 | 2.63 ± 0.386 | — |
| Dabrafenib, | 1 | h | 276 ± 17.2 | 225 ± 29.2 | — |
| 100 mg/kg, | 6 | h | 46.2 ± 5.05 | 32.8 ± 0.260 | 157 ± 12.3 |
| single dose | 12 | h | 6.74 ± 2.73 | 9.59 ± 1.68 | 19.2 ± 5.84 |
| | 24 | h | 0.725 ± 0.110 | 1.34 ± 0.369 | — |
| Compound | 1 | h | 20067 ± 1506 | 1650 ± 249 | — |
| 83, 10 | 6 | h | 10433 ± 338 | 1817 ± 354 | 6990 ± 166 |
| mg/kg, | 12 | h | 2423 ± 572 | 751 ± 128 | 1580 ± 273 |
| single dose | 24 | h | 172 | 57.6 ± 37.0 | — |
| Compound | 1 | h | 2446 ± 799 | 232 ± 75.6 | — |
| 93, 10 | 6 | h | 548 ± 28.3 | 129 ± 13.6 | 460 ± 32.7 |
| mg/kg, | 12 | h | 114 ± 25.6 | 50.6 ± 14.1 | 117 ± 30.2 |
| single dose | 24 | h | 2.85 ± 0.408 | 59.5 | — |
| Compound | 1 | h | 15100 ± 3559 | 816 ± 239 | — |
| 70, 10 | 6 | h | 8563 ± 2312 | 1529 ± 458 | 4363 ± 1423 |
| mg/kg, | 12 | h | 2105 ± 800 | 564 ± 206 | 921 ± 317 |
| single dose | 24 | h | 106 ± 10.8 | 21.9 ± 4.93 | — |
| Compound | 1 | h | 7743 ± 1179 | 513 ± 28.8 | — |
| 54, 10 | 6 | h | 3407 ± 703 | 931 ± 147 | 4470 ± 501 |
| mg/kg, | 12 | h | 605 ± 124 | 477 ± 125 | 650 ± 84.2 |
| single dose | 24 | h | 54.0 ± 9.50 | 29.8 ± 2.90 | — |

### Western Blotting Analysis Results

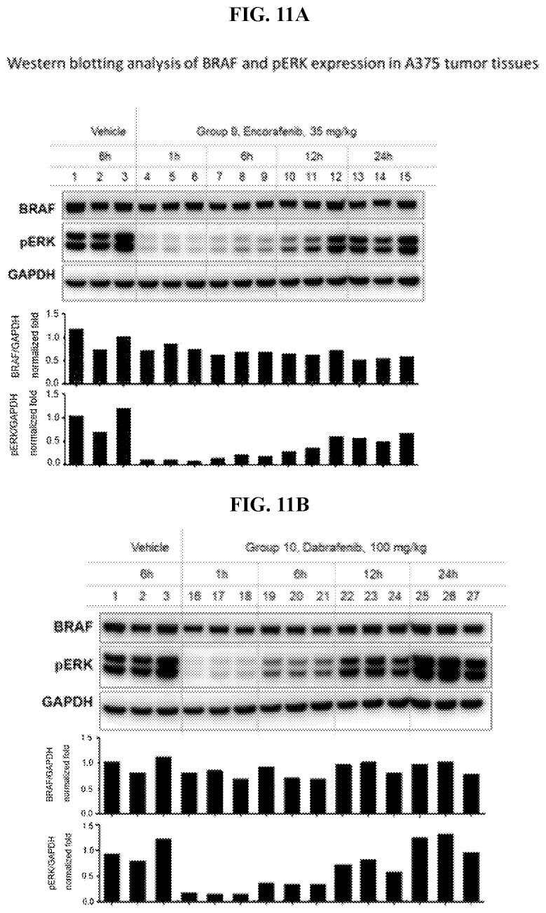

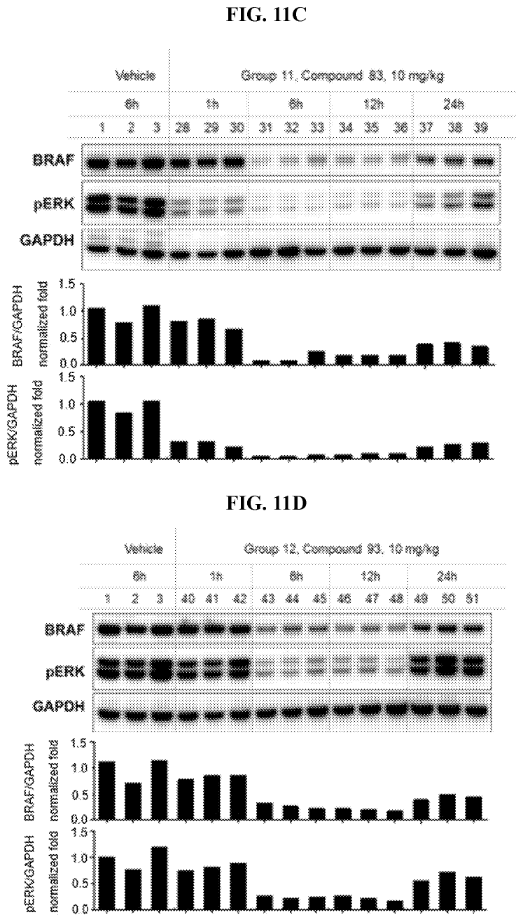

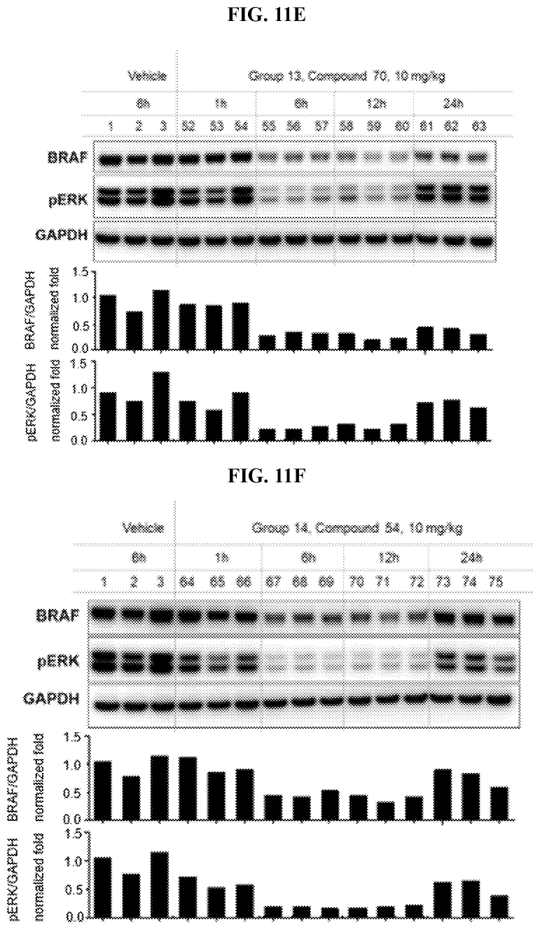

The results of BRAF and p-ERK expression in A375 tumors are shown in FIG. 11.

### Example 7 In vivo PKPD study and efficacy study of Compound 141, Compound 118, Compound 156, and Compound 180

### Method

For efficacy study, agents were tested in the A375 human melanoma xenografts. Compound 118, Compound 156, and Compound 180 were administered to BALB/c nude mice bearing A375 xenografts from day 0 and dosed p.o. twice per day (BID). Compound 141 was administered from day 0 and dosed p.o. twice per day (BID) or dosed p.o. daily (QD) from day 1. Compound 141, Compound 118, Compound 156, and Compound 180 were dosed at 10 mg/kg for 14 days. The known BRAF inhibitor Dabrafenib was used as a positive control. Dabrafenib was administered from day 1 and dosed p.o. daily (QD) for 14 days and dosed at 100 mg/kg.

For the PK and PD study, agents were administered to BALB/c nude mice bearing A375 human melanoma xenografts tumors. Dabrafenib, Compound 141, Compound 118, Compound 156, and Compound 180 were dosed p.o. once on day 1. Known BRAF inhibitor Dabrafenib was used as a positive control. Dabrafenib was dosed at 100 mg/kg. Compound 141, Compound 118, Compound 156, and Compound 180 were dosed at 10 mg/kg.

### Experimental Design

TABLE 16
Study Design for Efficacy Study

| | | | Weeks 1-3 | | | | |
|---|---|---|---|---|---|---|---|
| | | | Dose | Dosing | | Sample | Sample |
| Group | N^{a} | Treatment^{b} | (mg/kg) | Route | Schedule^{d} | Type^{d} | Collection^{d} |
| 1^{c} | 6 | Vehicle | — | p.o. | BID × 14 | — | — |
| | | | | | days | | |
| 2 | 6 | Dabrafenib | 100 | p.o. | QD × 14 | Plasma, | 6 h |
| | | | | | days | Liver | |
| 3 | 6 | Compound | 10 | p.o. | QD × 14 | Plasma, | 6 h |
| | | 141 | | | days | Liver | |
| 4 | 6 | Compound | 10 | p.o. | BID × 14 | Plasma, | 6 h |
| | | 141 | | | days | Liver | |
| 5 | 6 | Compound | 10 | p.o. | BID × 14 | Plasma, | 6 h |
| | | 118 | | | days | Liver | |
| 6 | 6 | Compound | 10 | p.o. | BID × 14 | Plasma, | 6 h |
| | | 156 | | | days | Liver | |
| 7 | 6 | Compound | 10 | p.o. | BID × 14 | Plasma, | 6 h |
| | | 180 | | | days | Liver | |

Note:
^{a}N: animal number; Dosing volume: adjusted dosing volume based on body weight 10 μL/g.
^{b}The formulations for vehicle, Compound 141, Compound 118, Compound 156, and Compound 180 were 20% PEG400 + 80% (25% SBE-β-CD in ddH₂O). The formulation for Dabrafenib was 10% PEG400 + 90% (11.1% HP-β-CD in ddH₂O).
^{c}The mice from Group 1 were euthanized on PG-D 14 without sample collection.
^{d}Plasma and liver samples were collected from groups 2-7 at 6 hours post last dose on PG-D 14 for PK analysis.

TABLE 17
Study Design for the PK and PD Study

| | | | | | | Time point | Time point | Time point |
|---|---|---|---|---|---|---|---|---|
| | | | Dose | Dosing | | for Plasma | for Tumor | for Liver |
| Group | N^{a} | Treatment^{b} | (mg/kg) | Route | Schedule | Collection^{c} | Collection^{d} | Collection^{c} |
| 8 | 3 | Vehicle | — | p.o. | Single | — | 6 h | — |
| | | | | | dose | | | |
| 9 | 12 | Dabrafenib | 100 | p.o. | Single | 1 h, 6 h, 12 | 1 h, 6 h, 12 | 6 h, 12 h; |
| | | | | | dose | h, 24 h; n = 3 | h, 24 h; n = 3 | n = 3 |
| 10 | 12 | Compound | 10 | p.o. | Single | 1 h, 6 h, 12 | 1 h, 6 h, 12 | 6 h, 12 h; |
| | | 141 | | | dose | h, 24 h; n = 3 | h, 24 h; n = 3 | n = 3 |
| 11 | 12 | Compound | 10 | p.o. | Single | 1 h, 6 h, 12 | 1 h, 6 h, 12 | 6 h, 12 h; |
| | | 118 | | | dose | h, 24 h; n = 3 | h, 24 h; n = 3 | n = 3 |
| 12 | 12 | Compound | 10 | p.o. | Single | 1 h, 6 h, 12 | 1 h, 6 h, 12 | 6 h, 12 h; |
| | | 156 | | | dose | h, 24 h; n = 3 | h, 24 h; n = 3 | n = 3 |
| 13 | 12 | Compound | 10 | p.o. | Single | 1 h, 6 h, 12 | 1 h, 6 h, 12 | 6 h, 12 h; |
| | | 180 | | | dose | h, 24 h; n = 3 | h, 24 h; n = 3 | n = 3 |

Note:
^{a}N: animal number; Dosing volume: adjusted dosing volume based on body weight 10 μL/g.
^{b}The formulations for vehicle, Compound 141, Compound 118, Compound 156, and Compound 180 were 20% PEG400 + 80% (25% SBE-β-CD in ddH₂O). The formulation for Dabrafenib was 10% PEG400 + 90% (11.1% HP-β-CD in ddH₂O).
^{c}Plasma and liver samples were collected at predetermined time points for PK analysis. Tumor samples were collected at predetermined time points for PK and WB analysis

### Materials

### Animals and Housing Condition

One hundred and seventeen female BALB/c nude mice procured from Shanghai Lingchang Biological Science and Technology Co., LTD between the ages of six to eight weeks and with body weight between eighteen and twenty-two grams were used for the study. A spare mouse was also procured.

The mice were kept in individual ventilation cages at constant temperature and humidity with 3 or 6 animals in each cage. The cage was kept between approximately 20 to 26° C. with a humidity of 40-70%. The cages were made of polycarbonate with a size of 375 mm×215 mm 180 mm and corn cob bedding which was changed twice per week.

The animals were fed irradiation sterilized dry granule food during the entire study period. The animals had free access to food and sterile drinking water. Cages were identified with the following information: number of animals, sex, strain, date received, treatment, study number, group number and the starting date of the treatment and each animal was marked by ear coding.

Test Articles

| Compound | Molecular Weight | Formula Weight |
|---|---|---|
| Dabrafenib | 519.56 | 519.56 |
| Compound 141 | 895.99 | 895.99 |
| Compound 118 | 856.95 | 856.95 |
| Compound 156 | 883.98 | 883.98 |
| Compound 180 | 866.99 | 866.99 |

### Reagents and Materials for PD

Reagents and Consumables

| No. | Reagents/Consumables | Vendor | Catalog # |
|---|---|---|---|
| 1 | RIPA Buffer | Sigma | R0278 |
| 2 | Phosphatase Inhibitor Cocktail 2 | Sigma | P5726 |
| 3 | Protease Inhibitor Cocktail | Roche | 04693124001 |
| 4 | Pierce ™ BCA Protein Assay Kit | Thermo | 23225 |
| | | Scientific | |
| 5 | NuPAGE ™ LDS Sample Buffer | Invitrogen | NP0007 |
| | (4×) | | |
| 6 | NuPAGE ™ Sample Reducing Agent | Invitrogen | NP0009 |
| | (10×) | | |
| 7 | NuPAGE ® MES SDS Running | Invitrogen | NP0002 |
| | Buffer (20×) | | |
| 8 | 20 × TBS | Bio-Serve | BS-P-15 |
| 9 | Tween 20 | Sigma | P2287 |
| 10 | Intercept ™ (TBS) Blocking Buffer | LI-COR | 927-60001 |
| 11 | PageRuler ™ Prestained Protein | Thermo | 26619 |
| | Ladder | Scientific | |
| 12 | iBlot ® 2 Transfer Stack Regular | Invitrogen | IB23001 |
| 13 | NuPAGE, Novex 4-12% Bis-Tris | Invitrogen | WG1403BOX |
| | Gel, 26 well | | |
| 14 | Bovine Serum Albumin | BBI life | A600332- |
| | | sciences | 0100 |
| 15 | SuperSignal ™ West Femto | Thermo | 34096 |
| | Maximum Sensitivity Substrate | Scientific | |
| 16 | Nonfat Milk | BD Difco | 232100 |
| 17 | SuperSignal West Pico | Thermo | 34078 |
| | Chemiluminescent Substrate | Scientific | |

1 × TBST: added 50 mL 20 × TBS to 949 mL H₂O, and then added 1 mL Tween 20, mixed well.
Running buffer: added 50 mL 20 × MES SDS Running Buffer to 950 mL H₂O, mixed well.

Instruments

| No. | Instruments | Vendor | Model # |
|---|---|---|---|
| 1 | Tissuelyser | Shanghai Jingxin | JXFSTPRP-CL |
| 2 | Electro- thermal | Boxun | HPX-9162 MBE |
| | incubator | | |
| 3 | SpectraMax | Molecular Devices | M5e |
| 4 | Dry baths | Hangzhou All sheng | MK-10 |
| | | Instruments Co., Ltd | |
| 5 | Xcell4 SureLock ® | Invitrogen | WR0100 |
| | Midi-cell | | |
| 6 | PowerPac ™ Basic | Bio-Rad | 1645050 |
| | Power Supply | | |
| 7 | Orbital Shaker | Qiling | TS-1 |
| 8 | iBlot ® 2 Gel Transfer | Invitrogen | iBlot 2 |
| | Device | | |
| 9 | Beads | Qiagen | 69989 |
| 10 | Amersham Imager 680 | GE Healthcare | AI680QC |
| 11 | Vortex Shaker | Kylin-Bell | Vortex-6 |
| 12 | Centrifuge | Eppendorf | 5424R |

Antibodies

| No. | Antibodies | Vendor | Catalog # |
|---|---|---|---|
| 1 | B-RAF (D9T6S) antibody | Cell Signaling | CST-14814 |
| | | Technology | |
| 2 | phospho-p44/42 MAPK (Erk1/2) | Cell Signaling | CST-9101 |
| | (Thr202/Tyr204) antibody | Technology | |
| 3 | GAPDH antibody | Cell Signaling | CST-5174 |
| | | Technology | |
| 4 | Goat Anti-Rabbit IgG H&L (HRP) | Abcam | ab6721 |

### Experimental Procedures

### Cell Culture

The A375 tumor cells were maintained in vitro in DMEM medium supplemented with 100 fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation. The details of reagents are shown in Table 18.

TABLE 18
Vendor and Cat No. of Cell Line and Reagents

| Cell Line/Reagent | Vendor | Catalog # |
|---|---|---|
| A375 | ATCC | CRL-1619 |
| DMEM | Gibco | 11995-065 |
| Penicillin-Streptomycin | Gibco | 15240-062 |
| 0.25% Trypsin-EDTA | Gibco | 25200-072 |
| FBS | Corning | 35-081-CV |
| DPBS | Corning | 21-031-CVC |
| Matrigel | Corning | 354234 |

### Tumor Inoculation

Each mouse was inoculated subcutaneously at the right flank with A375 tumor cells (5×10⁶) in 0.2 mL of PBS with Matrigel (PBS: Matrigel=1:1) supplemented for tumor development. Animals were grouped when the average tumor volume reached 246 mm³ for efficacy study and 390 mm³ for PKPD study on day 17 after tumor inoculation. The test articles administration and the animal numbers in each group are shown in the above experimental tables.

### Reagents Used in the Study

TABLE 19
Vendor and Cat No. of Reagents Used for Formulation Preparation

| Reagent | Vendor | Catalog # |
|---|---|---|
| PEG400 | Sigma | P3265 |
| SBE-β-CD | Kunshan Ruisike Chemical Materials | 10059859 |
| HP-β-CD | Energy chemical | E080016 |

### Vehicle Preparation

### Preparation of 25% SBE-β-CD in ddH₂O

50 g of SBE-β3-CD was accurately weighed into a 500 mL glass bottle. 150 mL volume of ddH₂O was then added and the mixture was stirred to obtain a clear solution. Then ddH₂O was added to a total volume of 200 mL and the mixture was mixed well.

### Preparation of 11.1% HP-β-CD in ddH₂O

22.2 g of HP-βC-CD was accurately weighed into a 500 mL glass bottle. 150 mL volume of ddH₂O was then added and the mixture was stirred to obtain a clear solution. Then ddH₂O was added to a total volume of 200 mL and the mixture was mixed well.

### Testing Article Formulation Preparation

TABLE 20
Formulation of Dabrafenib Preparation

| | | | | | Volume of | |
|---|---|---|---|---|---|---|
| | | | | Volume of | 11.1% HP-β- | |
| | | | | PEG400 to | CD in | |
| | | Conc. of | Compound | be added | ddH2O to be | Dosing |
| Compound | Dose | solution | amount | (mL) | added (mL) | Solution |
| ID | (mg/kg) | (mg/mL) | (mg) | (10%) | (90%) | observations |
| Dabrafenib^{a} | 100 | 10 | 53.62 | 0.536 | 4.821 | Suspension |

Note:
The formulation for efficacy study was prepared once every three days. The formulation for PKPD study was prepared freshly.

TABLE 21
Formulations of C4T's Compounds Preparation

| | | | | Volume of | Volume of | |
|---|---|---|---|---|---|---|
| | | | | PEG400 to | 25% SBE-β- | |
| | | Conc. of | Compound | be added | CD to be | Dosing |
| Compound^{a} | Dose | solution | amount | (mL) | added (mL) | Solution |
| ID | (mg/kg) | (mg/mL) | (mg) | (20%) | (80%) | observations |
| Compound 141 | 10 | 1 | 16.21 | 3.236 | 12.945 | Solution |
| Compound 118 | 10 | 1 | 10.27 | 2.037 | 8.149 | Solution |
| Compound 156 | 10 | 1 | 10.39 | 2.045 | 8.179 | Suspension |
| Compound 180 | 10 | 1 | 9.95 | 1.974 | 7.897 | Solution |

Note:
^{a}The formulations for efficacy study were prepared once every three days. The formulations for PKPD study were prepared freshly.

### Observations

At the time of routine monitoring, the animals were checked daily for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset. Animals that were observed to be in a continuing deteriorating condition or their tumor size exceeding 3,000 mm³ were euthanized prior to death or before reaching a comatose state.

### Tumor Measurements and the Endpoints

Prior to the onset of drug treatment, mice were measured for tumor size in two dimensions using a caliper, and the tumor volume (mm³) was calculated using formula V=0.5 a×b² where a and b are the long and short diameters of the tumor in mm, respectively. Mice were randomized into different treatment groups based on the tumor volume.

The major endpoint was to see if the tumor growth could be delayed or mice could be cured. The tumor size was then used for calculations of T/C value. The T/C value (in percent) is an indication of antitumor effectiveness; T and C are the mean volumes of the treated and control groups, respectively on specified day.

TGI was calculated for each group using the formula: TGI (%)=[1−(Tᵢ−T₀)/(Cᵢ−C₀)]×100; Tᵢ is the average tumor volume of a treatment group on a given day, T₀ is the average tumor volume of the treatment group on the day of treatment start, Cᵢ is the average tumor volume of the vehicle control group on the same day with Tᵢ, and C₀ is the average tumor volume of the vehicle group on the day of treatment start.

### Experimental Endpoint and Sample Collection

### Plasma Sample Preparation

The plasma samples were collected at predetermined time points for PK analysis. The mice were euthanized at predetermined time points post the treatment. The blood samples were collected into the 1.5 mL Eppendorf tubes containing 15 μL of 0.5 M EDTA-K2. And then the anticoagulant blood samples were centrifuged at 2,500 g at 4° C. for 15 minutes. The plasma samples were separated into 2 aliquots. The 1^{st} was used for PK analysis, and the 2^{nd} served as backup. The samples were stored at −80° C. before bioanalysis.

### Tumor Sample Preparation

The tumor samples were collected at predetermined time points for PK and WB analysis. The tumor was dissected out and split into 3 parts. The 1^{st} part was used for PK analysis, the 2^{nd} part was used for WB analysis and the last one served as backup for study. The samples were transferred into pre-labeled tubes and quickly froze in liquid nitrogen within 2 minutes after dissection. The samples were stored at −80° C. before bioanalysis.

### Liver Sample Preparation

Liver samples were collected at predetermined time points for PK analysis. The left lobe of the liver was dissected out and split into 2 parts. The 1^{st} part was used for PK analysis and the 2^{nd} part served as the backup for the study. The samples were transferred into pre-labeled tubes and quickly froze in liquid nitrogen within 2 minutes after dissection. The samples were stored at −80° C. before bioanalysis.

### Pharmacokinetics Assay

### Pharmacokinetics Assay for Efficacy Study

The harvested plasma and liver samples were processed for pharmacokinetics analysis by LC-MS/MS with a concentration range of 0.500~500 ng/mL for Dabrafenib in plasma and liver homogenate, 20.0~20,000 ng/mL for Compound 141, Compound 180 and Compound 118 in plasma and liver, 10.0~10,000 ng/mL for Compound 156 in plasma and liver.

### Pharmacokinetics Assay for PKPD Study

The harvested plasma and liver samples were processed for pharmacokinetics analysis by LC-MS/MS with a concentration range of 0.500-1000 ng/mL for Dabrafenib, Compound 118 and Compound 180 in plasma, 0.100~200 ng/ml for Dabrafenib in Liver and tumor homogenate, 50.0~50,000 ng/mL for Compound 141 in plasma and Liver homogenate, 1.00-1000 ng/ml for Compound 141 in tumor homogenate, 2.00~2000 ng/mL for Compound 118 and Compound 180 in Liver and 0.200~200 ng/ml in tumor homogenate, 1.00~2000 ng/mL for Compound 156 in plasma, 0.500-1000 ng/ml in liver and 0.500-1,000 ng/ml in tumor homogenate.

### Western Blotting Assay

### Protein Extraction & Protein Quantification

The previously snap-frozen cells were removed from the −80° C. freezer and then cut up to about 30-100 mg of tissue per sample. The tissues were then placed in 2 mL microcentrifuge tube and 400 μL of RIPA Buffer was then added. The RIPA Buffer contained 1% Protease Inhibitor Cocktail and 1% Phosphatase Inhibitor Cocktail 2.

The tumors were then ground with a freeze grinder at 50 Hz for five minutes and then the tissue lysates were kept on ice for thirty minutes. The lysates were centrifuged at 15,000 rpm at 4° C. for ten minutes and then the supernatants were gently transferred to a new microcentrifuge tube kept on ice.

The protein concentration was measured with a Pierce™ BCA Protein Assay Kit. Then the samples were diluted, according to the result of BCA protein quantification, to the same final concentration (at 2 μg/μL) using RIPA buffer plus 4× LDS Sample Buffer and 10× Sample Reducing Agent. The samples were then heated at 100° C. for ten minutes. The denatured samples were then used for Western blotting or kept in the −80° C. refrigerator for long term storage.

### Western Blotting

The protein samples were loaded to NuPAGE® Novex 4-12% Bis-Tris gel with 10 μL per slot. The electrophoresis with MES running buffer was ran at 80 V for 30 minutes and then at 120 V for 90 minutes. Then protein was transferred to NC (nitrocellulose) membranes with iBlot®2 Gel Transfer Device, P3 for 7 minutes. Afterwards, the membranes were cut, the interest membranes parts were kept and washed with 5-10 mL 1× TBST three times for 5 minutes each. Then the non-specific protein was blocked by 5-10 mL 1× TBST with 5% nonfat milk at room temperature for 1 hour. The membrane was then washed with 5-10 mL 1× TBST three times for 5 minutes each. The membrane was then incubated with 5-10 mL diluted primary antibody (BRAF antibody was diluted at a ratio of 1:1000, phospho-p44/42 MAPK antibody was diluted at a ratio of 1:2000 and GAPDH antibody was diluted at a ratio of 1:2000) in 1× TBST with 5% BSA at 4° C. with gentle agitation overnight. Next, the membrane was washed with 5-10 mL 1× TBST three times for 10 minutes each. The resulting membrane was incubated with 5-10 mL HRP-conjugated secondary antibody (generally 1:3000) with gentle agitation for 1 hour at room temperature. Then the membrane was washed with 5-10 mL 1× TBST three times for 10 minutes each. Afterwards, the HRP substrate from West Femto Maximum Sensitivity kit was added to the NC membranes and chemiluminescence was detected by AI680QC.

### Quantification of Band Intensity

Intensity of individual bands were quantified using Image Studio Lite Ver 5.2 software.

### Statistical Analysis

Band intensity of individual bands was obtained for analysis. Protein expression was quantitated in relation to a reference protein, and then the normalized folds of protein expression in each sample were ratios to vehicle group.

The normalized data were interpreted by GraphPad Prism. Statistical analysis of difference in the protein expression of interests among the groups was conducted.

### Data Management and Statistical Analyses

A one-way ANOVA followed by Dunnett's multiple comparison test was performed to compare tumor volume among vehicle group and treatment groups. All data were analyzed using GraphPad Prism, **** indicates p<0.0001.

### Results

### Results for Efficacy Study

### Body Weight Change

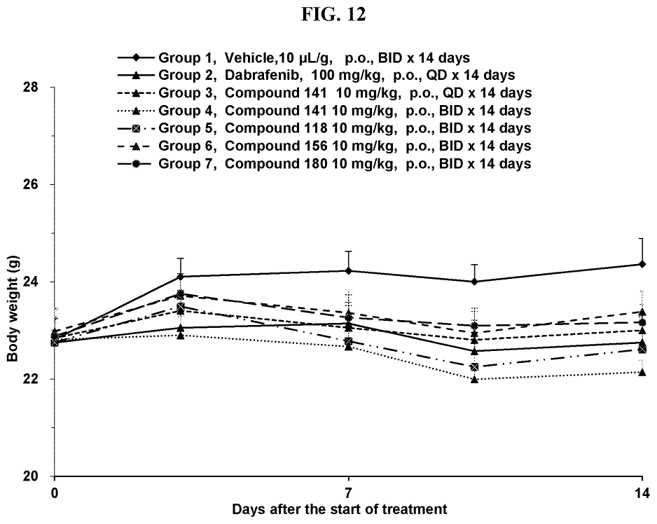

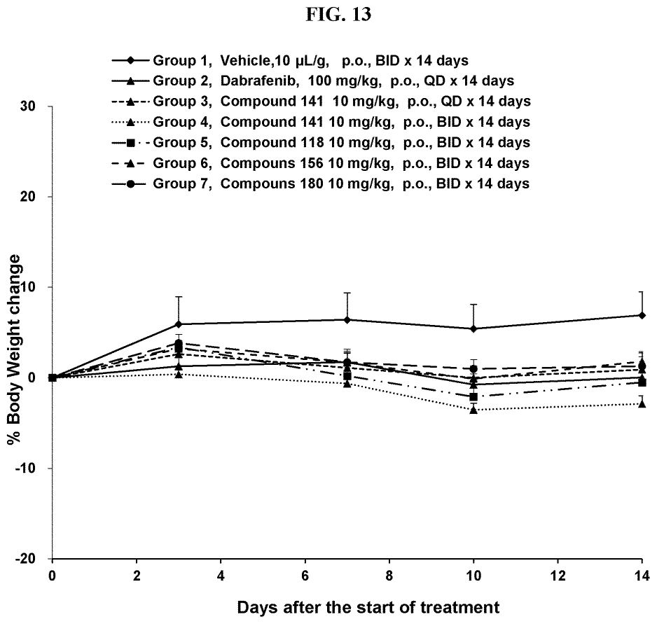

Animal body weight was monitored regularly as an indirect measurement of toxicity. Body weight changes after administration of Dabrafenib, Compound 141, Compound 118, Compound 156 and Compound 180 are shown in FIG. 12 and FIG. 13.

### Tumor Growth Curve

Tumor growth curves are shown in FIG. 14.

### Tumor Volume Trace

Mean tumor size over time in female BALB/c nude mice bearing A375 human melanoma xenografts is shown in Table 22.

TABLE 22
Tumor Volume Trace Over Time (mm³)^{a}

| | Days | | | | |
|---|---|---|---|---|---|
| Group# | 0 | 3 | 7 | 10 | 14 |
| Group 1, Vehicle, | 246 ± 11^{a} | 513 ± 20 | 820 ± 45 | 1036 ± 78 | 1329 ± 82 |
| p.o., BID × 14 days | | | | | |
| Group 2, Dabrafenib, | 246 ± 10 | 394 ± 20 | 515 ± 30 | 608 ± 21 | 741 ± 40 |
| 100 mg/kg, p.o., QD × 14 days | | | | | |
| Group 3, Compound 141, | 246 ± 12 | 275 ± 9 | 138 ± 13 | 81 ± 9 | 40 ± 6 |
| 10 mg/kg, p.o., QD × 14 days | | | | | |
| Group 4, Compound 141, | 246 ± 15 | 181 ± 15 | 109 ± 13 | 59 ± 6 | 32 ± 4 |
| 10 mg/kg, p.o., BID × 14 days | | | | | |
| Group 5, Compound 118, | 247 ± 14 | 326 ± 20 | 192 ± 20 | 117 ± 13 | 81 ± 8 |
| 10 mg/kg, p.o., BID × 14 days | | | | | |
| Group 6, Compound 156, | 246 ± 13 | 203 ± 16 | 111 ± 4 | 68 ± 3 | 34 ± 5 |
| 10 mg/kg, p.o., BID × 14 days | | | | | |
| Group 7, Compound 180, | 247 ± 16 | 319 ± 22 | 222 ± 18 | 151 ± 16 | 96 ± 11 |
| 10 mg/kg, p.o., BID × 14 days | | | | | |

Note:
Data are shown as Mean ± SEM.

### Tumor Growth Inhibition Analysis

Tumor growth inhibition of Dabrafenib, Compound 141, Compound 118, Compound 156 and Compound 180 in the treatment of female BALB/c nude mice bearing A375 xenografts was calculated based on tumor volume measured on PG-D14. TGI and T/C were calculated respectively, and the data are detailed in Table 23.

TABLE 23
Tumor Growth Inhibition Analysis (T/C and TGI)^{a}

| | Tumor Size | | | |
|---|---|---|---|---|
| | (mm³)^{a} | T/C^{b} | TGI^{c} | p |
| Treatment | on PG-D14 | (%) | (%) | value^{d} |
| Group 1, Vehicle, | 1329 ± 82 | / | / | / |
| p.o., BID × 14 days | | | | |
| Group 2, Dabrafenib, | 741 ± 40 | 55.8 | 54.3 | **** |
| 100 mg/kg, p.o., QD × 14 days | | | | |
| Group 3, Compound 141, | 40 ± 6 | 3.0 | 119.0 | **** |
| 10 mg/kg, p.o., QD × 14 days | | | | |
| Group 4, Compound 141, | 32 ± 4 | 2.4 | 119.8 | **** |
| 10 mg/kg, p.o., BID × 14 days | | | | |
| Group 5, Compound 118, | 81 ± 8 | 6.1 | 115.3 | **** |
| 10 mg/kg, p.o., BID × 14 days | | | | |
| Group 6, Compound 156, | 34 ± 5 | 2.6 | 119.6 | **** |
| 10 mg/kg, p.o., BID × 14 days | | | | |
| Group 7, Compound 180, | 96 ± 11 | 7.2 | 113.9 | **** |
| 10 mg/kg, p.o., BID × 14 days | | | | |

Note:
^{a}Data are shown as Mean ± SEM.
^{b}Antitumor activity (T/C) was determined by dividing the average tumor volume for treated group (T) by the average tumor volume for control group (C).
^{c}Tumor Growth Inhibition (TGI) was calculated using the formula: TGI (%) = (1 − (T − T₀)/(C − C₀))*100, where T = mean tumor size at the terminal end of treatment group; T₀ = mean tumor size at day 0 of treatment group; C = mean tumor size at the terminal end of control group; and C₀ = mean tumor size at day 0 of control group.
^{d}A one-way ANOVA followed by Dunnett's multiple comparison test was performed to compare tumor volume among vehicle group and treatment groups. All data were analyzed using GraphPad Prism, **** indicates p < 0.0001.

### Pharmacokinetics Results

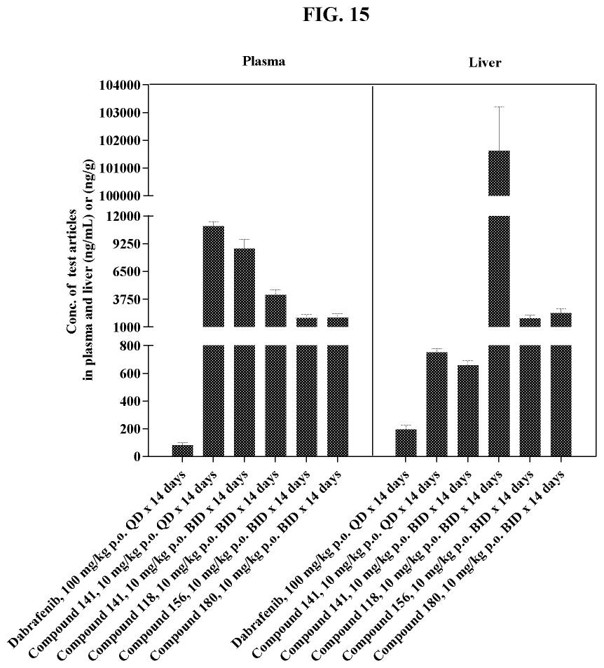

The concentration of Dabrafenib, Compound 141, Compound 118, Compound 156, and Compound 180 in the treatment of female BALB/c nude mice bearing A375 xenografts are shown in Table 24 and FIG. 15.

TABLE 24
Mean Concentration of Test Articles in Plasma and Liver

| | | Concentration | Concentration |
|---|---|---|---|
| | Sample | in Plasma | in Liver |
| Group | time | (ng/mL) ± SEM | (ng/g) ± SEM |
| Group 2, Dabrafenib, | 6 h | 80.5 ± 17.3 | 195 ± 32.1 |
| 100 mg/kg, p.o., QD × 14 days | | | |
| Group 3, Compound 141, | 6 h | 10972 ± 474 | 7505 ± 286 |
| 10 mg/kg, p.o., QD × 14 days | | | |
| Group 4, Compound 141, | 6 h | 8747 ± 907 | 6568 ± 341 |
| 10 mg/kg, p.o., BID × 14 days | | | |
| Group 5, Compound 118, | 6 h | 4163 ± 503 | 101617 ± 1595 |
| 10 mg/kg, p.o., BID × 14 days | | | |
| Group 6, Compound 156, | 6 h | 1874 ± 359 | 1840 ± 305 |
| 10 mg/kg, p.o., BID × 14 days | | | |
| Group 7, Compound 180, | 6 h | 1922 ± 363 | 2398 ± 425 |
| 10 mg/kg, p.o., BID × 14 days | | | |

### Results for PK and PD Study

### Pharmacokinetics Results

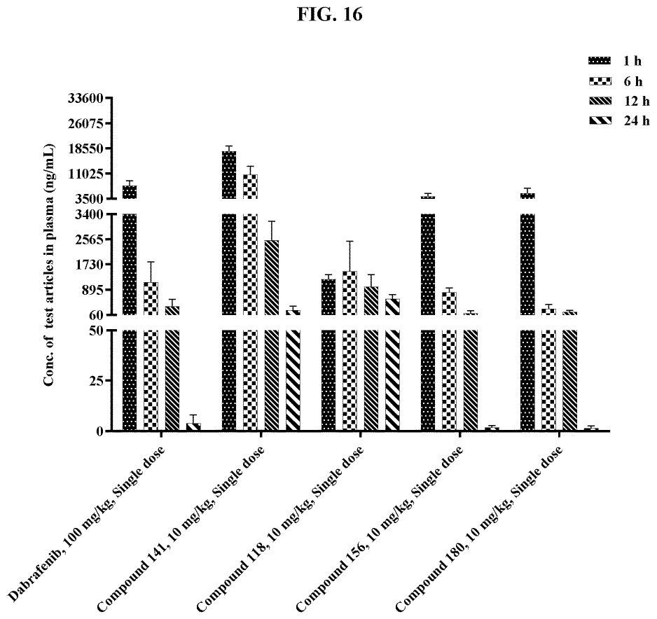

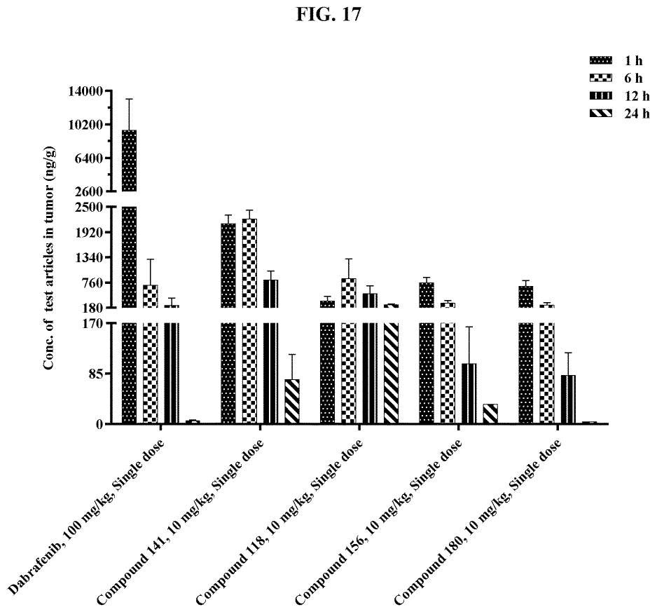

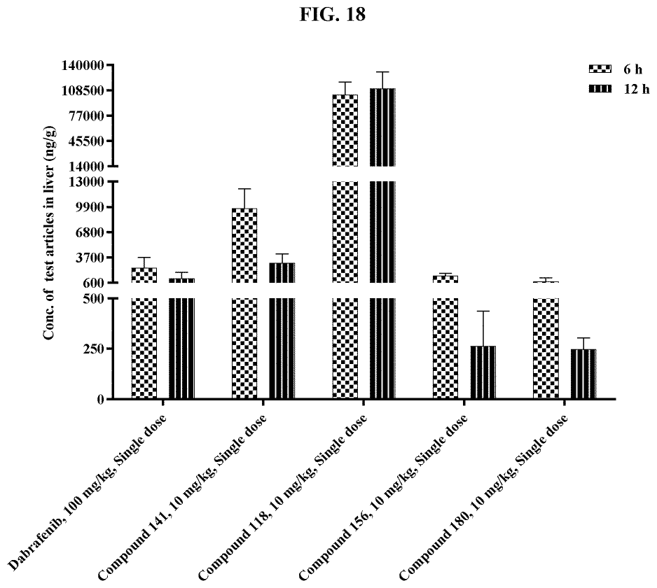

The concentration of Dabrafenib, Compound 141, Compound 118, Compound 156, and Compound 180 in the treatment of female BALB/c nude mice bearing A375 xenografts are shown in Table 25 and FIG. 16-18.

TABLE 25
Mean Concentration of Test Articles in Plasma, Tumor and Liver

| | | | Concentration | Concentration | Concentration |
|---|---|---|---|---|---|
| | Sample | | in Plasma | in Tumor | in Liver |
| Group | time | | (ng/mL) ± SEM | (ng/g) ± SEM | (ng/g) ± SEM |
| Group 9, | 1 | h | 7370 ± 865 | 9517 ± 2050 | — |
| Dabrafenib, | 6 | h | 1137 ± 390 | 702 ± 341 | 2453 ± 737 |
| 100 mg/kg, p.o., | 12 | h | 336 ± 137 | 242 ± 96.0 | 1107 ± 453 |
| Single dose | 24 | h | 3.88 ± 2.43 | 5.53 ± 0.951 | — |
| Group 10, | 1 | h | 17700 ± 924 | 2123 ± 109 | — |
| Compound 141, | 6 | h | 10677 ± 1423 | 2230 ± 114 | 9733 ± 1382 |
| 10 mg/kg, p.o., | 12 | h | 2533 ± 365 | 818 ± 121 | 3047 ± 618 |
| Single dose | 24 | h | 219 ± 74.6 | 75.4 ± 24.3 | — |
| Group 11, | 1 | h | 1240 ± 90.2 | 345 ± 57.4 | — |
| Compound 118, | 6 | h | 1501 ± 574 | 855 ± 257 | 102933 ± 9269 |
| 10 mg/kg, p.o., | 12 | h | 985 ± 234 | 509 ± 101 | 110933 ± 11984 |
| Single dose | 24 | h | 595 ± 81.8 | 254 ± 7.80 | — |
| Group 12, | 1 | h | 4127 ± 545 | 758 ± 66.9 | — |
| Compound 156, | 6 | h | 805 ± 85.2 | 295 ± 30.3 | 1460 ± 170 |
| 10 mg/kg, p.o., | 12 | h | 111 ± 46.3 | 102 ± 35.6 | 262 ± 100 |
| Single dose | 24 | h | 1.78 ± 0.554 | 33.5 | — |
| Group 13, | 1 | h | 5110 ± 867 | 680 ± 72.8 | — |
| Compound 180, | 6 | h | 260 ± 84.3 | 245 ± 34.7 | 712 ± 282 |
| 10 mg/kg, p.o., | 12 | h | 155 ± 27.8 | 82.6 ± 21.8 | 247 ± 33.7 |
| Single dose | 24 | h | 1.54 ± 0.588 | 3.89 | — |

### Western Blotting Analysis Results

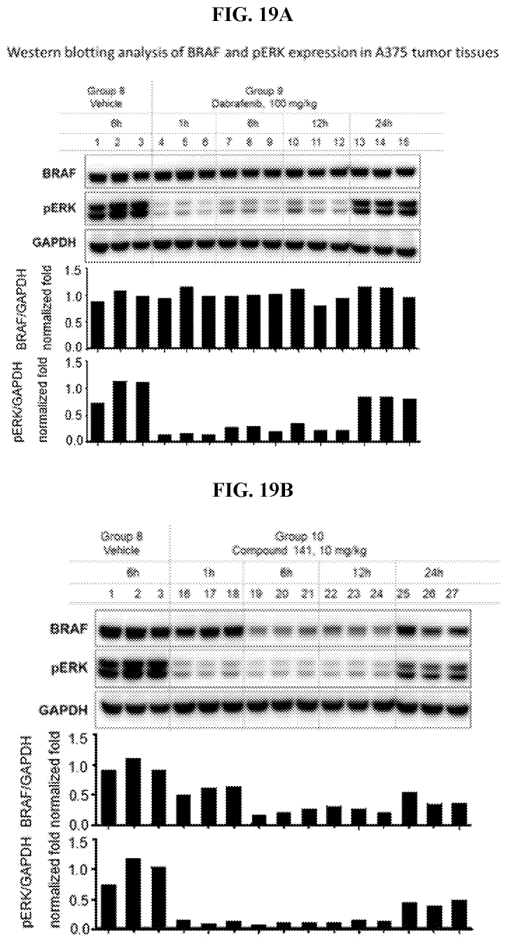

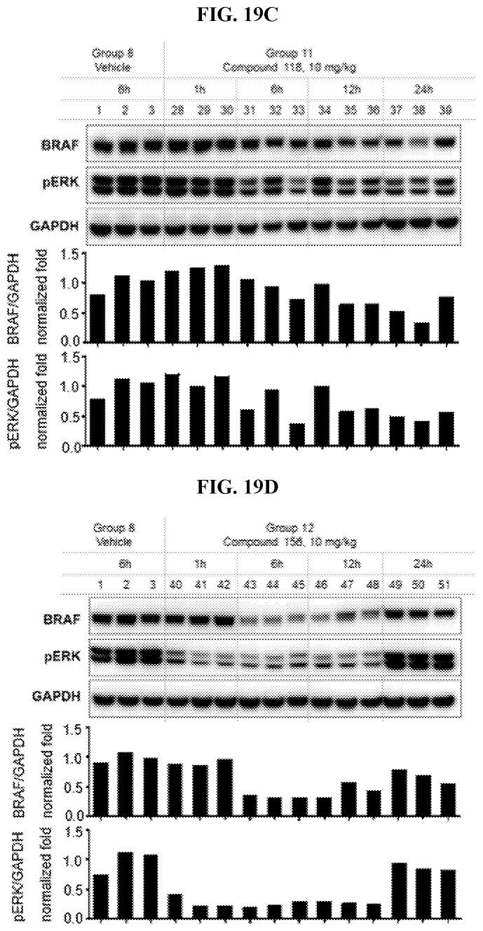

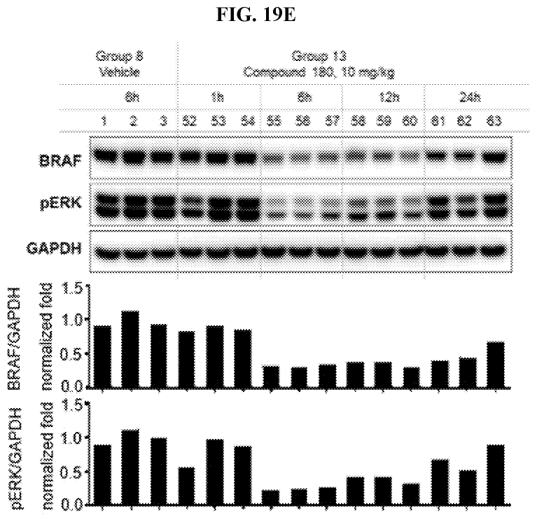

The results of BRAF and p-ERK expression in A375 tumors are shown in FIG. 19.

### Example 8 In vivo PKPD study and efficacy study of Compound 8, Compound 26, Compound 38, and Compound 39

### Method

For efficacy study, agents were tested in the A375 human melanoma xenografts model. Compound 8, Compound 26, and Compound 38 were administered to BALB/c nude mice bearing A375 xenografts from day 0 and dosed p.o. twice per day (BID). Compound 8 and Compound 26 were dosed at 10 mg/kg for 21 days. Compound 38 was dosed at 10 mg/kg for 10 days. The known BRAF inhibitor encorafenib was used as a positive control. encorafenib was administered from day 1 and dosed p.o. daily (QD) for 21 days and dosed at 35 mg/kg.

For the PK and PD study, agents were administered to BALB/c nude mice bearing A375 human melanoma xenografts tumors. encorafenib, Compound 8, Compound 26, Compound 38, and Compound 39 were dosed p.o. once on day 1. Encorafenib was dosed at 35 mg/kg, Compound 8, Compound 26, Compound 38, and Compound 39 were dosed at 10 mg/kg. This study was non-GLP.

### Experimental Design

TABLE 26
Study Design for Efficacy Study

| | | | Weeks 1-3 | | | Plasma and |
|---|---|---|---|---|---|---|
| | | | Dose | Dosing | | Liver Collection |
| Group | N^{a} | Treatment^{b} | (mg/kg) | Route | Schedule^{d} | at 6 h |
| 1 | 6 | Vehicle | — | p.o. | BID × 21 | — |
| | | | | | days | |
| 2 | 6 | Encorafenib | 35 | p.o. | QD × 21 | #2-1, #2-3, |
| | | | | | days | #2-3 |
| 3 | 6 | Compound | 10 | p.o. | BID × 21 | #3-1, #3-2, |
| | | 8 | | | days | #3-3 |
| 4 | 6 | Compound | 10 | p.o. | BID × 21 | #4-1, #4-2, |
| | | 26 | | | days | #4-3 |
| 5^{c} | 6 | Compound | 10 | p.o. | BID × 10 | #5-1, #5-2, |
| | | 38 | | | days | #5-3 |

Note:
^{a}N: animal number; Dosing volume: adjusted dosing volume based on body weight 10 μL/g.
^{b}The formulation for vehicle, Compound 8, Compound 26 and Compound 38 were 20% PEG400 + 80% (25% SBE-β-CD in ddH₂O). The formulation for encorafenib was 0.5% CMC-Na + 0.5% Tween-80 in ddH₂O.
^{c}Mice from group 5 were euthanized on PG-D 10, plasma and liver samples of mice #5-1, #5-2 and #5-3 were collected at 6 h post last dose for PK analysis.
^{d}The remaining mice from Groups 2-4 were euthanized on PG-D 21. Plasma and liver samples were collected from the mice #2-1, #2-2 and #2-3 of Group 2, mice #3-1, #3-2 and #3-3 of Group 3, and mice #4-1, #4-2 and #4-3 of Group 4 at 6 h post last dose on PG-D 21 for PK analysis.

TABLE 27
Study Design for PK and PD Study

| | | Treat- | Dose | Dosing | Sched- | Sample |
|---|---|---|---|---|---|---|
| Group | N^{a} | ment^{b} | (mg/kg) | Route | ule | collection^{c} |
| 6 | 3 | Vehicle | — | p.o. | Single | 6 h; n = 3 |
| | | | | | dose | |
| 7 | 12 | Encorafenib | 35 | p.o. | Single | 1 h, 6 h, 12 h, |
| | | | | | dose | 24 h; n = 3 |
| 8 | 12 | Compound | 10 | p.o. | Single | 1 h, 6 h, 12 h, |
| | | 8 | | | dose | 24 h; n = 3 |
| 9 | 12 | Compound | 10 | p.o. | Single | 1 h, 6 h, 12 h, |
| | | 26 | | | dose | 24 h; n = 3 |
| 10 | 12 | Compound | 10 | p.o. | Single | 1 h, 6 h, 12 h, |
| | | 38 | | | dose | 24 h; n = 3 |
| 11 | 12 | Compound | 10 | p.o. | Single | 1 h, 6 h, 12 h, |
| | | 39 | | | dose | 24 h; n = 3 |

Note:
^{a}N: animal number; Dosing volume: adjusted dosing volume based on body weight 10 μL/g.
^{b}The formulation for vehicle, Compound 8, Compound 26, Compound 38 and Compound 39 were 20% PEG400 + 80% (25% SBE-β-CD in ddH₂O). The formulation for encorafenib was 0.5% CMC-Na + 0.5% Tween-80 in ddH₂O.

Plasma and liver samples were collected at predetermined time points for PK analysis. Tumor samples were collected at predetermined time points for PK and WB analysis.

### Animals and Housing Condition

Nineteen-three female BALB/c nude mice procured from Shanghai Lingchang Biological Science and Technology Co., LTD between the ages of six to eight weeks and with body weight between eighteen and twenty-two grams were used for the study. A spare mouse was also procured.

The mice were kept in individual ventilation cages at constant temperature and humidity with 3 or 6 animals in each cage. The cage was kept between approximately 20 to 26° C. with a humidity of 40-70%. The cages were made of polycarbonate with a size of 375 mm×215 mm×180 mm and corn cob bedding which was changed twice per week.

The animals were fed irradiation sterilized dry granule food during the entire study period. The animals had free access to food and sterile drinking water. Cages were identified with the following information: number of animals, sex, strain, date received, treatment, study number, group number and the starting date of the treatment and each animal was marked by ear coding.

Test Articles

| Compound | Molecular Weight | Formula Weight |
|---|---|---|
| Encorafenib | 540.01 | 540.01 |
| Compound 8 | 882.98 | 882.98 |
| Compound 26 | 911.97 | 911.97 |
| Compound 38 | 934.97 | 934.97 |
| Compound 39 | 934.97 | 934.97 |

### Reagents and Materials for PD

Reagents and Consumables

| No. | Reagents/Consumables | Vendor | Catalog # |
|---|---|---|---|
| 1 | RIPA Buffer | Sigma | R0278 |
| 2 | Phosphatase Inhibitor Cocktail 2 | Sigma | P5726 |
| 3 | Protease Inhibitor Cocktail | Roche | 04693124001 |
| 4 | Pierce ™ BCA Protein Assay Kit | Thermo | 23225 |
| | | Scientific | |
| 5 | NuPAGE ™ LDS Sample Buffer | Invitrogen | NP0007 |
| | (4×) | | |
| 6 | NuPAGE ™ Sample Reducing Agent | Invitrogen | NP0009 |
| | (10×) | | |
| 7 | NuPAGE ® MES SDS Running | Invitrogen | NP0002 |
| | Buffer (20×) | | |
| 8 | 20 × TBS | Bio-Serve | BS-P-15 |
| 9 | Tween 20 | Sigma | P2287 |
| 10 | Intercept ™ (TBS) Blocking Buffer | LI-COR | 927-60001 |
| 11 | PageRuler ™ Prestained Protein | Thermo | 26616 |
| | Ladder | Scientific | |
| 12 | iBlot ® 2 Transfer Stack Regular | Invitrogen | IB23001 |
| 13 | NuPAGE, Novex 4-12% Bis-Tris | Invitrogen | WG1402BOX |
| | Gel, 20 well | | |
| 14 | Bovine Serum Albumin | BBI life | A600332- |
| | | sciences | 0100 |
| 15 | SuperSignal ™ West Femto | Thermo | 34096 |
| | Maximum Sensitivity Substrate | Scientific | |
| 16 | Nonfat Milk | BD Difco | 232100 |
| 17 | SuperSignal West Pico | Thermo | 34078 |
| | Chemiluminescent Substrate | Scientific | |

1 × TBST: added 50 mL 20 × TBS to 949 mL H₂O, and then added 1 mL Tween 20, mixed well.
Running buffer: added 50 mL 20 × MES SDS Running Buffer to 950 mL H₂O, mixed well.

Instruments

| No. | Instruments | Vendor | Model # |
|---|---|---|---|
| 1 | Tissuelyser | Shanghai Jingxin | JXFSTPRP-CL |
| 2 | Electro- thermal | Boxun | HPX-9162 MBE |
| | incubator | | |
| 3 | SpectraMax | Molecular Devices | M5e |
| 4 | Dry baths | Hangzhou All sheng | MK-10 |
| | | Instruments Co., Ltd | |
| 5 | Xcell4 SureLock ® | Invitrogen | WR0100 |
| | Midi-cell | | |
| 6 | PowerPac ™ Basic | Bio-Rad | 1645050 |
| | Power Supply | | |
| 7 | Orbital Shaker | Qiling | TS-1 |
| 8 | iBlot ® 2 Gel Transfer | Invitrogen | iBlot 2 |
| | Device | | |
| 9 | Beads | Qiagen | 69989 |
| 10 | Amersham Imager 680 | GE Healthcare | AI680QC |
| 11 | Vortex Shaker | Kylin-Bell | Vortex-6 |
| 12 | Centrifuge | Eppendorf | 5424R |

Antibodies

| No. | Antibodies | Vendor | Catalog # |
|---|---|---|---|
| 1 | B-RAF (D9T6S) antibody | Cell Signaling | CST-14814 |
| | | Technology | |
| 2 | phospho-p44/42 MAPK (Erk1/2) | Cell Signaling | CST-9101 |
| | (Thr202/Tyr204) antibody | Technology | |
| 3 | GAPDH antibody | Cell signal | CST-5174 |
| | | technology | |
| 4 | Goat Anti-Rabbit IgG H&L | Abcam | ab6721 |
| | (HRP) | | |

### Experimental Methods and Procedures

### Cell Culture

The A375 tumor cells were maintained in vitro in DMEM medium supplemented with 10 fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation. The details of reagents are shown in Table 28.

TABLE 28
Vendor and Cat No. of Cell Line and Reagents

| Cell Line/Reagent | Vendor | Catalog # |
|---|---|---|
| A375 | ATCC | CRL-1619 |
| DMEM | Gibco | 11995-065 |
| Penicillin-Streptomycin | Gibco | 15240-062 |
| 0.25% Trypsin-EDTA | Gibco | 25200-072 |
| FBS | Corning | 35-081-CV |
| DPBS | Corning | 21-031-CVC |
| Matrigel | Corning | 354234 |

### Tumor Inoculation

Each mouse was inoculated subcutaneously at the right flank with A375 cells (5×10⁶) in 0.2 mL of PBS supplemented with Matrigel (PBS:Matrigel=1:1) for tumor development. Animals were grouped for treatment 16 days after tumor inoculation when the average tumor volume reached 234 mm³ for efficacy study and 382 mm³ for PKPD study. The test articles administration and the animal numbers in each group are shown in the above experimental tables.

### Reagents Used in the Study

TABLE 29
Vendor and Cat No. of Reagents Used for Formulation Preparation

| Reagent | Vendor | Catalog # |
|---|---|---|
| PEG400 | Sigma | P3265 |
| SBE-β-CD | Kunshan Ruisike Chemical Materials | 10059859 |
| CMC-Na | Sigma | C4888 |
| Tween 80 | Sigma | P4780 |

### Vehicle Preparation

Preparation of 0.5% CMC-Na and 0.500 Tween-80 in ddH₂O
1. Accurately weighed 1.0 g CMC-Na into a 500 mL glass bottle.
2. Added 160 mL ddH₂O and 1.000 mL Tween 80, stirred to obtain a clear solution.
3. Added ddH₂O to 200 mL, mixed well.
   Preparation of 25% SBECD in ddH₂O
1) Accurately weighed 50 g of SBE-C-CD into a 500 mL glass bottle.
2) Added 150 mL volume of ddH₂O, stirred to obtain a clear solution.
3) Added ddH₂O to 200 mL and mixed well.

### Testing Article Formulation Preparation

TABLE 30
Formulation of Encorafenib Preparation

| | | | | Volume of 0.5% | |
|---|---|---|---|---|---|
| | | | | CMC-Na and | |
| | | | | 0.5% Tween-80 | |
| | | Conc. of | Compound | in ddH₂O to | |
| Compound | Dose | solution | amount | be added | Dosing Solution |
| ID | (mg/kg) | (mg/mL) | (mg) | (mL) | observations |
| Encorafenib^{a} | 35 | 3.5 | 19.19 | 5.431 | Suspension |

Note:
^{a}Dosing solutions were prepared once every three days.

TABLE 31
Formulations of C4T's Compounds Preparation

| | | | | Volume of | Volume of | |
|---|---|---|---|---|---|---|
| | | | | PEG400 to | 25% SBE-β- | |
| | | Conc. of | Compound | be added | CD to be | Dosing |
| Compound^{a} | Dose | solution | amount | (mL) | added (mL) | Solution |
| ID | (mg/kg) | (mg/mL) | (mg) | (20%) | (80%) | observations |
| Compound 8 | 10 | 1 | 10.53 | 2.103 | 8.411 | Solution |
| Compound 26 | 10 | 1 | 10.25 | 2.030 | 8.120 | Solution |
| Compound 38 | 10 | 1 | 4.49 | 0.893 | 3.570 | Solution |
| Compound 39 | 10 | 1 | 3.72 | 0.737 | 2.948 | Solution |

Note:
^{a}The formulations for Compound 8 and Compound 26 were prepared once every three days. The formulations for Compound 38 and Compound 39 were prepared daily.

### Observations

At the time of routine monitoring, the animals were checked daily for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset. Animals that were observed to be in a continuing deteriorating condition or their tumor size exceeding 3000 mm³ were euthanized prior to death or before reaching a comatose state.

### Tumor Measurements and the Endpoints

Prior to the onset of drug treatment, mice were measured for tumor size in two dimensions using a caliper, and the tumor volume (mm³) was calculated using formula V=0.5 a×b² where a and b are the long and short diameters of the tumor in mm, respectively. Mice were randomized into different treatment groups based on the tumor volume.

The major endpoint was to see if the tumor growth could be delayed or mice could be cured. The tumor size was then used for calculations of T/C value. The T/C value (in percent) is an indication of antitumor effectiveness; T and C are the mean volumes of the treated and control groups, respectively on specified day.

TGI was calculated for each group using the formula: TGI (%)=[1−(Tᵢ−T₀)/(Cᵢ−C₀)]×100; Tᵢ is the average tumor volume of a treatment group on a given day, T₀ is the average tumor volume of the treatment group on the day of treatment start, Cᵢ is the average tumor volume of the vehicle control group on the same day with Tᵢ, and C₀ is the average tumor volume of the vehicle group on the day of treatment start.

### Experimental Endpoint and Sample Collection

### Plasma Sample Preparation

The plasma samples were collected at predetermined time points for PK analysis. For non-termination plasma collection, blood samples were collected into the 1.5 mL Eppendorf tubes containing 3 μL of 0.5 M EDTA-K2. For termination plasma collection, the mice were euthanized at predetermined time points post the treatment and the blood samples were collected into the 1.5 mL Eppendorf tubes containing 15 μL of 0.5 M EDTA-K2. And then the anticoagulant blood samples were centrifuged at 2,500 g at 4° C. for 15 minutes. The plasma samples were separated into 2 aliquots. The 1^{st} was used for PK analysis, and the 2^{nd} served as backup. The samples were stored at −80° C. before bioanalysis.

### Tumor Sample Preparation

The tumor samples were collected at predetermined time points for PK and WB analysis. The tumor was dissected out and split into 3 parts. The 1^{st} part was used for PK analysis, the 2^{nd} part was used for WB analysis and the last one served as backup for study. The samples were transferred into pre-labeled tubes and quickly froze in liquid nitrogen within 2 minutes after dissection. The samples were stored at −80° C. before bioanalysis.

### Liver Sample Preparation

Liver samples were collected at predetermined time points for PK analysis. The left lobe of the liver was dissected out and split into 2 parts. The 1^{st} part was used for PK analysis and the 2^{nd} part served as the backup for the study. The samples were transferred into pre-labeled tubes and quickly froze in liquid nitrogen within 2 minutes after dissection. The samples were stored at −80° C. before bioanalysis.

### Pharmacokinetics Assay

### Pharmacokinetics Assay for Efficacy Study

The harvested plasma and liver samples were processed for pharmacokinetics analysis by LC-MS/MS with a concentration range of 2.0~2000 ng/mL for encorafenib in plasma and liver homogenate, 10.0-10,000 ng/mL for Compound 8 and Compound 38 in plasma and liver homogenate, 20.0~20,000 ng/mL for Compound 26 in plasma and liver homogenate.

### Pharmacokinetics Assay for PKPD Study

The harvested plasma and liver samples were processed for pharmacokinetics analysis by LC-MS/MS with a concentration range of 0.200~200 ng/mL for encorafenib in plasma and liver homogenate, and 0.100-100 ng/ml in tumor homogenate, 1.00-1000 ng/mL for Compound 8 in plasma and 0.500~500 ng/ml in tumor and liver homogenate, 50.0~50,000 ng/mL for Compound 26 and Compound 38 in plasma, 5.0~5000 ng/ml in liver and 2.00~2000 ng/ml in tumor homogenate, 1.00-1000 ng/mL for Compound 39 in plasma and 0.500~500 ng/mL in liver and tumor homogenate.

### Western Blotting Assay

### Protein Extraction & Protein Quantification

The previously snap-frozen cells were removed from the −80° C. freezer and then cut up to about 30-100 mg of tissue per sample. The tissues were then placed in 2 mL microcentrifuge tube and 400 μL of RIPA Buffer was then added. The RIPA Buffer contained 1% Protease Inhibitor Cocktail and 1% Phosphatase Inhibitor Cocktail 2.

The tumors were then ground with a freeze grinder at 50 Hz for five minutes and then the tissue lysates were kept on ice for thirty minutes. The lysates were centrifuged at 15,000 rpm at 4° C. for ten minutes and then the supernatants were gently transferred to a new microcentrifuge tube kept on ice.

The protein concentration was measured with a Pierce™ BCA Protein Assay Kit. Then the samples were diluted, according to the result of BCA protein quantification, to the same final concentration (at 2 μg/μL) using RIPA buffer plus 4× LDS Sample Buffer and 10× Sample Reducing Agent. The samples were then heated at 100° C. for ten minutes. The denatured samples were then used for Western blotting or kept in the −80° C. refrigerator for long term storage.

### Western Blotting

The protein samples were loaded to NuPAGE® Novex 4-12% Bis-Tris gel with 10 μL per slot. The electrophoresis with MES running buffer was ran at 80 V for 30 minutes and then at 120 V for 90 minutes. Then protein was transferred to NC (nitrocellulose) membranes with iBlot®2 Gel Transfer Device, P3 for 7 minutes. Afterwards, the membranes were cut, the interest membranes parts were kept and washed with 5-10 mL 1× TBST once for 5 minutes. Then the non-specific protein was blocked by 5-10 mL LI-COR Blocking Buffer at room temperature for 1 hour. The membrane was then washed with 5-10 mL 1× TBST two times for 5 minutes each. The membrane was then incubated with 5-10 mL diluted primary antibody (BRD9 antibody was diluted at a ratio of (1:1000), phospho-p44/42 MAPK antibody was diluted at a ratio of (1:2000) and GAPDH antibody was diluted at a ratio of (1:2000) in LI-COR Blocking Buffer containing 0.1% Tween 20 at 4° C. with gentle agitation overnight. Next, the membrane was washed with 5-10 mL 1× TBST three times for 10 minutes each. The resulting membrane was incubated with 5-10 mL Goat Anti-Rabbit IgG H&L (HRP) (generally 1:10000) in LI-COR Blocking Buffer containing 0.1% Tween 20 with gentle agitation for 1 hour at room temperature. Then the membrane was washed with 5-10 mL 1× TBST three times for 10 minutes each. Afterwards, the membranes was washed with 5-10 mL 1× TBS two times for 5 minutes each and chemiluminescence was detected by Amersham Imager 680.

### Quantification of Band Intensity

Intensity of individual bands were quantified using Image Studio Lite Ver 5.2 software.

### Statistical Analysis

Band intensity of individual bands was obtained for analysis. Protein expression was quantitated in relation to a reference protein, and then the normalized folds of protein expression in each sample were ratios to vehicle group.

The normalized data were interpreted by GraphPad Prism. Statistical analysis of difference in the protein expression of interests among the groups was conducted.

### Data Management and Statistical Analyses

The T-test was performed to compare tumor volume between Group 1 and Group 5. A one-way ANOVA followed by Dunnett's multiple comparison test was performed to compare tumor volume among vehicle group (Group 1) and treatment groups (Group 2-Group 4). All data were analyzed using GraphPad Prism, ns indicates p>0.05, * indicates p<0.05, ** indicates p<0.01, *** indicates p<0.001 and **** indicates p<0.0001.

### Results

### Results for Efficacy Study

### Body Weight Change

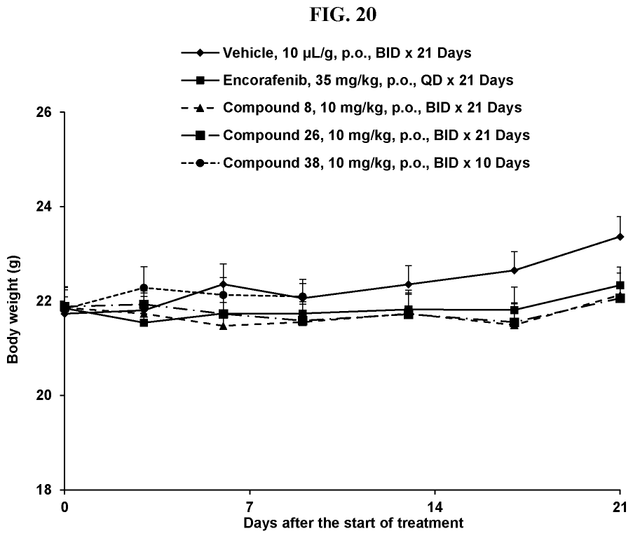

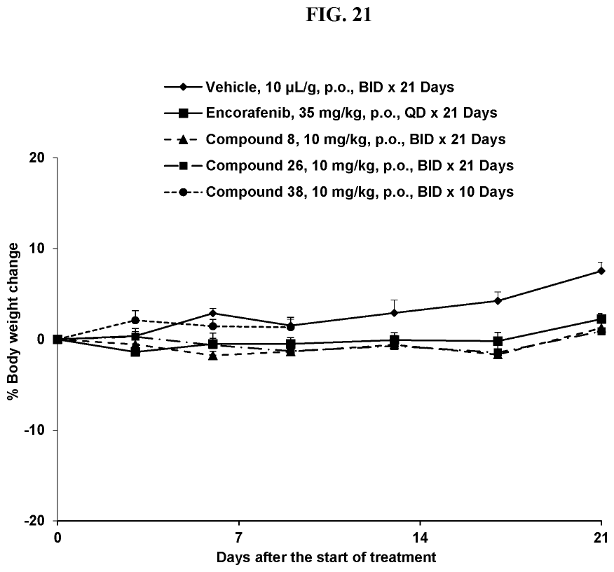

Animal body weight was monitored regularly as an indirect measurement of toxicity. Body weight changes after administration of vehicle, encorafenib, Compound 8, Compound 26 and Compound 38 are shown in FIG. 20 and FIG. 21.

### Tumor Growth Curve

Tumor growth curves are shown in FIG. 22.

### Tumor Volume Trace

Mean tumor size over time in female BALB/c nude mice bearing A375 human melanoma xenografts is shown in Table 32.

TABLE 32
Tumor Volume Trace Over Time (mm³)^{a}

| | Group# | | | | |
|---|---|---|---|---|---|
| | | Group 2, | Group 3, | Group 4, | Group 5, |
| | Group 1, | Encorafenib, | Compound 8, | Compound 26, | Compound 38, |
| | Vehicle, p.o., | 35 mg/kg, p.o., | 10 mg/kg, p.o., | 10 mg/kg, p.o., | 10 mg/kg, p.o., |
| Days | BID × 21 days | QD × 21 days | BID × 21 days | BID × 21 days | BID × 10 days |
| 0 | 234 ± 7^{a} | 234 ± 6 | 234 ± 7 | 234 ± 7 | 234 ± 7 |
| 3 | 383 ± 25 | 329 ± 17 | 185 ± 6 | 148 ± 10 | 134 ± 7 |
| 6 | 610 ± 44 | 347 ± 21 | 105 ± 9 | 57 ± 4 | 67 ± 7 |
| 9 | 813 ± 61 | 251 ± 20 | 57 ± 4 | 33 ± 3 | 35 ± 3^{b} |
| 13 | 1103 ± 80 | 194 ± 16 | 32 ± 4 | 21 ± 2 | — |
| 17 | 1410 ± 104 | 194 ± 15 | 28 ± 3 | 18 ± 1 | — |
| 21 | 1698 ± 117 | 254 ± 19 | 22 ± 3 | 16 ± 2 | — |

Note:
^{a}Data are shown as Mean ± SEM.
^{b}The mice from group 5 were euthanized on PG-D10.

### Tumor Growth Inhibition Analysis

Tumor growth inhibition of encorafenib, Compound 8 and Compound 26 in the treatment of female BALB/c nude mice bearing A375 xenografts was calculated based on tumor volume measured on PG-D21, and Compound 38 was calculated based on tumor volume measured on PG-D9. The data are detailed in Table 33.

TABLE 33
Tumor Growth Inhibition Analysis (T/C and TGI)^{a}

| | Tumor | Tumor | | | |
|---|---|---|---|---|---|
| | Size | Size | | | |
| | (mm³)^{a} | (mm³)^{a} | T/C^{b} | TGI^{c} | p |
| Treatment | on PG-D9 | on PG-D21 | (%) | (%) | valued |
| Group 1, Vehicle, p.o., | 813 ± 61 | 1698 ± 117 | / | / | / |
| BID × 21 days | | | | | |
| Group 2, Encorafenib, 35 mg/kg, | — | 254 ± 19 | 14.94 | 98.67 | **** |
| p.o., QD × 21 days | | | | | |
| Group 3, Compound 8, 10 mg/kg, | — | 22 ± 3 | 1.29 | 114.48 | **** |
| p.o., BID × 21 days | | | | | |
| Group 4, Compound 26, 10 | — | 16 ± 2 | 0.97 | 114.89 | **** |
| mg/kg, p.o., BID × 21 days | | | | | |
| Group 5, Compound 38, 10 | 35 ± 3 | — | 4.30 | 134.38 | **** |
| mg/kg, p.o., BID × 10 days | | | | | |

Note:
^{a}Data are shown as Mean ± SEM.
^{b}Antitumor activity (T/C) was determined by dividing the average tumor volume for treated group (T) by the average tumor volume for control group (C).
^{c}Tumor Growth Inhibition (TGI) was calculated using the formula: TGI (%) = (1 − (T − T₀)/(C − C₀))*100, where T = mean tumor size at the terminal end of treatment group; T₀ = mean tumor size at day 0 of treatment group; C = mean tumor size at the terminal end of control group; and C₀ = mean tumor size at day 0 of control group.
dT-test was performed to compare tumor volume between Group 1 and Group 5. A one-way ANOVA followed by Dunnett's multiple comparison test was performed to compare tumor volume among vehicle group (Group 1) and treatment groups (Group 2-Group 4). All data were analyzed using GraphPad Prism, *** indicates p < 0.001 and **** indicates p < 0.0001.
e. The mice from group 5 were euthanized on PG-D10.

### Pharmacokinetics Results

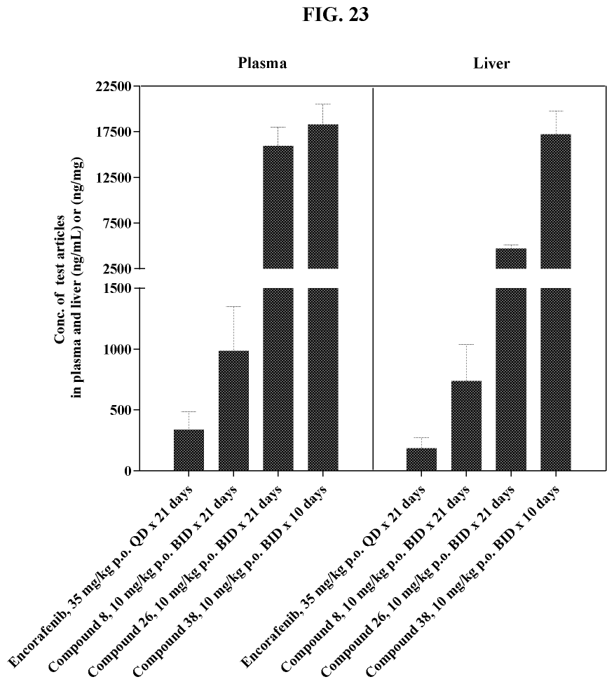

The concentration of encorafenib, Compound 8, Compound 26 and Compound 38 in the treatment of female BALB/c nude mice bearing A375 xenografts are shown in Table 34 and FIG. 23.

TABLE 34
Mean Concentration of Encorafenib, Compound 8,
Compound 26 and Compound 38 in Plasma and Liver

| | | Concentration | Concentration |
|---|---|---|---|
| | Sample | in Plasma | in Liver |
| Group | time | (ng/mL) ± SEM | (ng/g) ± SEM |
| Group 2, | 6 h | 336 ± 150 | 186 ± 87.2 |
| Encorafenib, 35 mg/kg, | | | |
| p.o., QD × 21 days | | | |
| Group 3, | 6 h | 984 ± 364 | 738 ± 299 |
| Compound 8, 10 mg/kg, | | | |
| p.o., BID × 21 days | | | |
| Group 4, | 6 h | 15967 ± 2061 | 4720 ± 367 |
| Compound 26, 10 mg/kg, | | | |
| p.o., BID × 21 days | | | |
| Group 5, | 6 h | 18267 ± 2239 | 17200 ± 2572 |
| Compound 38, 10 mg/kg, | | | |
| p.o., BID × 10 days | | | |

### Results for PK and PD Study

### Pharmacokinetics Results

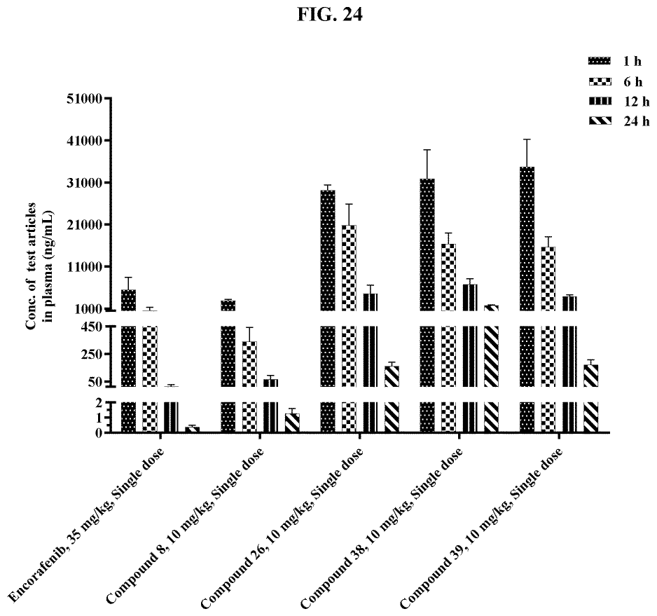

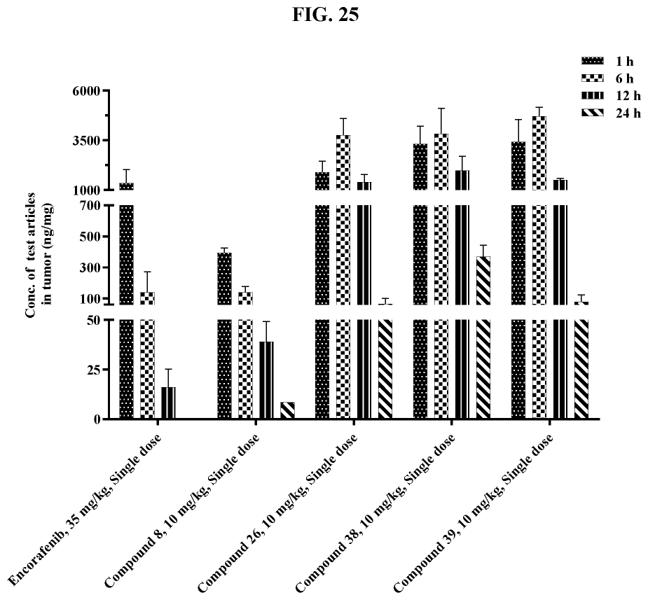

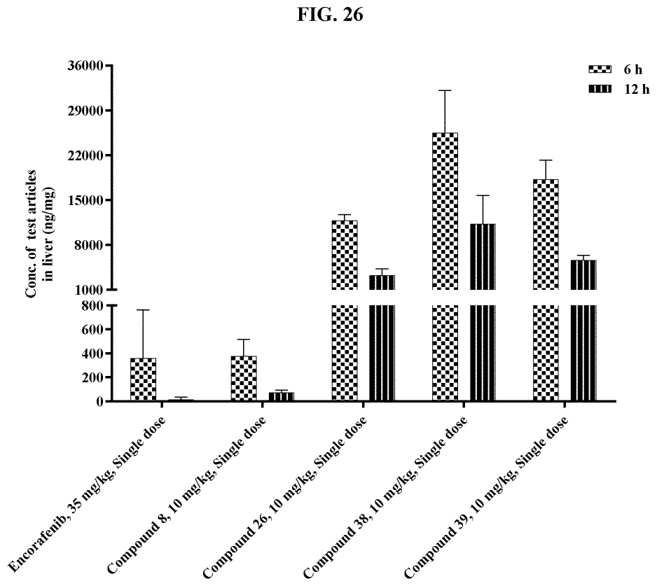

The concentration of Encorafenib, Compound 8, Compound 26, Compound 38 and Compound 39 in the treatment of female BALB/c nude mice bearing A375 xenografts are shown in Table 35 and FIG. 24-26.

TABLE 35
Mean Concentration of Test Articles in Plasma, Tumor and Liver

| | | | Concen- | Concen- | Concen- |
|---|---|---|---|---|---|
| | | | tration | tration | tration |
| | | | in Plasma | in Tumor | in Liver |
| | Sample | | (ng/mL) ± | (ng/g) ± | (ng/g) ± |
| Group | time | | SEM | SEM | SEM |
| Group 7, | 1 | h | 5513 ± 1697 | 1350 ± 391 | — |
| Encorafenib, | 6 | h | 585 ± 446 | 139 ± 76.2 | 362 ± 231 |
| 35 mg/kg, p.o., | 12 | h | 11.3 ± 8.5 | 16.1 ± 10.9 | 16.0 ± 5.3 |
| Single dose | 24 | h | 0.368 ± 0.082 | NA^{a} | — |
| Group 8, | 1 | h | 2936 ± 149 | 394 ± 17.6 | — |
| Compound 8, | 6 | h | 339 ± 60.3 | 139 ± 21.7 | 379 ± 79.8 |
| 10 mg/kg, p.o., | 12 | h | 64.7 ± 16.8 | 39.1 ± 5.77 | 74.8 ± 11.3 |
| Single dose | 24 | h | 1.27 ± 0.2 | 8.52 | — |
| Group 9, | 1 | h | 29133 ± 733 | 1900 ± 320 | — |
| Compound 26, | 6 | h | 20833 ± 2933 | 3767 ± 478 | 11800 ± 551 |
| 10 mg/kg, p.o., | 12 | h | 4597 ± 1143 | 1401 ± 226 | 3250 ± 601 |
| Single dose | 24 | h | 162 ± 15.3 | 64.9 ± 19.9 | — |
| Group 10, | 1 | h | 31967 ± 3959 | 3317 ± 523 | — |
| Compound 38, | 6 | h | 16367 ± 1495 | 3833 ± 736 | 25467 ± 3818 |
| 10 mg/kg, p.o., | 12 | h | 6807 ± 795 | 1987 ± 415 | 11243 ± 2595 |
| Single dose | 24 | h | 1797 ± 63.3 | 369 ± 42.6 | — |
| Group 11, | 1 | h | 34700 ± 3785 | 3423 ± 648 | — |
| Compound 39, | 6 | h | 15700 ± 1358 | 4713 ± 256 | 18200 ± 1735 |
| 10 mg/kg, p.o., | 12 | h | 3973 ± 202 | 1510 ± 43.6 | 5610 ± 455 |
| Single dose | 24 | h | 171 ± 20.8 | 79.3 ± 25.3 | — |

Note:
^{a}NA, not available.

### Western Blotting Analysis Results

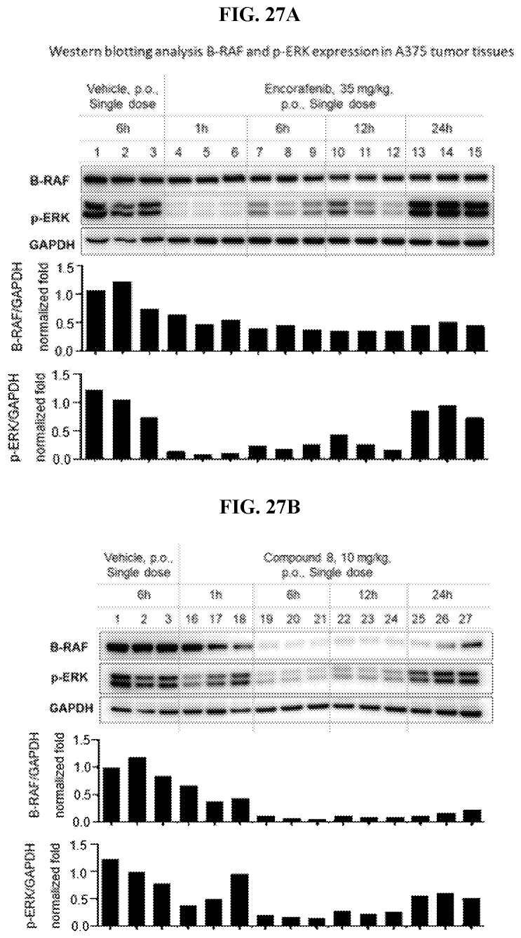

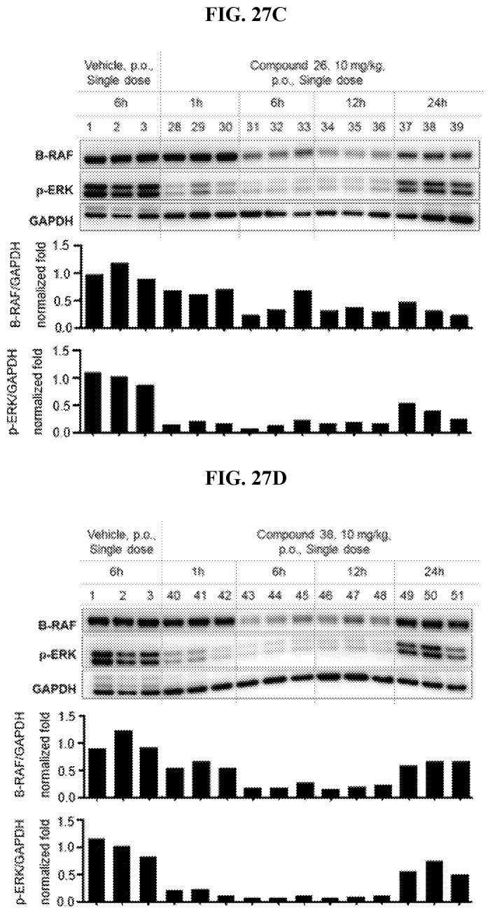

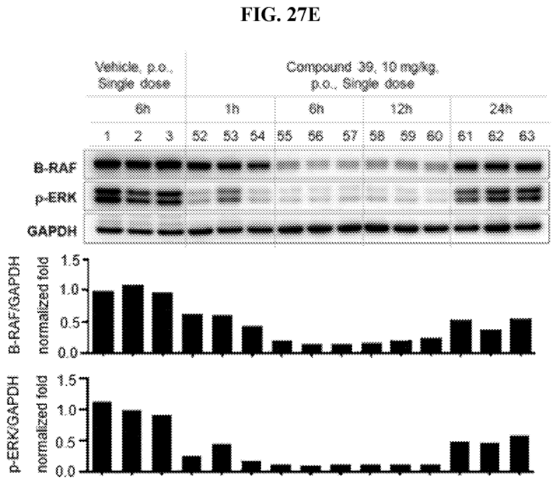

The results of BRAF and p-ERK expression in A375 tumors are shown in FIG. 27.

### Example 9 In vivo PKPD studies and efficacy studies of Compound 218, Compound 157, Compound 133, Compound 144, Compound 198, and Compound 204

### Method

For efficacy study Part I, agents were tested in the A375 human melanoma xenografts. Compound 218 and Compound 157 were administered to BALB/c nude mice bearing A375 xenografts from day 0 and dosed twice a day (BID) for 14 days. Compound 218 and Compound 157 were dosed at 10 mg/kg via p.o.. Dabrafenib was administered from day 1 daily (QD) for 14 days and dosed at 100 mg/kg via p.o..

For efficacy study Part II, agents were tested in the A375 human melanoma xenografts model. Compound 133, Compound 144 and Compound 198 were administered to BALB/c nude mice bearing A375 xenografts from day 0 and dosed twice a day (BID) for 14 days. Compound 133, Compound 144 and Compound 198 were dosed at 10 mg/kg via p.o..

For the PK and PD study Part I, agents were administered to BALB/c nude mice bearing A375 human melanoma xenografts tumors. Dabrafenib, Compound 218 and Compound 157 were dosed via p.o. once on day 1. Dabrafenib was dosed at 100 mg/kg. Compound 218 and Compound 157 were dosed at 10 mg/kg.

For the PK and PD study Part II, agents were administered to BALB/c nude mice bearing A375 human melanoma xenografts tumors. Compound 133, Compound 144 and Compound 198 were dosed at 10 mg/kg via p.o. once on day 1.

For the PK and PD study Part III, agent was administered to BALB/c nude mice bearing A375 human melanoma xenografts tumors. Compound 204 were dosed at 10 mg/kg via p.o. once on day 1.

This study was non-GLP.

### Experimental Design

TABLE 36A
Study Design for Efficacy Study Part I

| | | | Weeks | | | | |
|---|---|---|---|---|---|---|---|
| | | | 1-2 Dose | Dosing | | Sample | Sample |
| Group | N^{a} | Treatment^{b} | (mg/kg) | Route | Schedule | Type^{c} | Collection |
| 1 | 6 | Vehicle | — | p.o. | BID × 14 days | — | — |
| 2 | 6 | Dabrafenib | 100 | p.o. | QD × 14 days | Plasma, Liver | 6 h |
| 3 | 6 | Compound 218 | 10 | p.o. | BID × 14 days | Plasma, Liver | 6 h |
| 4 | 6 | Compound 157 | 10 | p.o. | BID × 14 days | Plasma, Liver | 6 h |

Note:
^{a}N: animal number; Dosing volume: adjusted dosing volume based on body weight 10 μL/g.
^{b}The formulations for vehicle, Compound 218 and Compound 157 were 20% PEG400 + 80% (25% SBE-β-CD in ddH₂O). The formulation for Dabrafenib was 10% PEG400 + 90% (11.1% HP-β-CD in ddH₂O).
^{c}Plasma and liver samples were collected from groups 2-4 at 6 hours post last dose on PG-D14 for PK analysis.

TABLE 36B
Study Design for Efficacy Study Part II

| | | | Weeks | | | | |
|---|---|---|---|---|---|---|---|
| | | | 1-2 Dose | Dosing | | Sample | Sample |
| Group | N^{a} | Treatment^{b} | (mg/kg) | Route | Schedule | Type^{c} | Collection |
| 5 | 6 | Vehicle | — | p.o. | BID × 14 days | — | — |
| 6 | 6 | Compound 133 | 10 | p.o. | BID × 14 days | Plasma, Liver | 6 h |
| 7 | 6 | Compound 144 | 10 | p.o. | BID × 14 days | Plasma, Liver | 6 h |
| 8 | 6 | Compound 198 | 10 | p.o. | BID × 14 days | Plasma, Liver | 6 h |

Note:
^{a}N: animal number; Dosing volume: adjusted dosing volume based on body weight 10 μL/g.
^{b}The formulations for vehicle, Compound 133, Compound 144 and Compound 198 were 20% PEG400 + 80% (25% SBE-β-CD in ddH₂O).
^{c}Plasma and liver samples were collected from groups 2-4 at 6 hours post last dose on PG-D14 for PK analysis.

TABLE 37
Study Design for the PK and PD Study Part I

| | | | | | | Time point | Time point | Time point |
|---|---|---|---|---|---|---|---|---|
| | | | Dose | Dosing | | for Plasma | for Tumor | for Liver |
| Group | N^{a} | Treatment^{b} | (mg/kg) | Route | Schedule | Collection^{c} | Collection^{d} | Collection^{c} |
| 10 | 3 | Vehicle | — | p.o. | Single | — | 6 h, n = 3 | — |
| | | | | | dose | | | |
| 11 | 12 | Dabrafenib | 100 | p.o. | Single | 1 h, 6 h, 12 h, | 1 h, 6 h, 12 h, | 6 h, 12 h; n = 3 |
| | | | | | dose | 24 h; n = 3 | 24 h; n = 3 | |
| 12 | 12 | Compound | 10 | p.o. | Single | 1 h, 6 h, 12 h, | 1 h, 6 h, 12 h, | 6 h, 12 h; n = 3 |
| | | 218 | | | dose | 24h; n = 3 | 24h; n = 3 | |
| 13 | 12 | Compound | 10 | p.o. | Single | 1 h, 6 h, 12 h, | 1 h, 6 h, 12 h, | 6 h, 12 h; n = 3 |
| | | 157 | | | dose | 24h; n = 3 | 24h; n = 3 | |

Note:
^{a}N: animal number; Dosing volume: adjusted dosing volume based on body weight 10 μL/g.
^{b}The formulations for vehicle, Compound 218 and Compound 157 were 20% PEG400 + 80% (25% SBE-β-CD in ddH₂O). The formulation for Dabrafenib was 10% PEG400 + 90% (11.1% HP-β-CD in ddH₂O).
^{c}Plasma and liver samples were collected at predetermined time points for PK analysis.
^{d}Tumor samples were collected at predetermined time points for PK and WB analysis.

TABLE 38
Study Design for the PK and PD Study Part II

| | | | | | | Time point | Time point | Time point |
|---|---|---|---|---|---|---|---|---|
| | | | Dose | Dosing | | for Plasma | for Tumor | for Liver |
| Group | N^{a} | Treatment^{b} | (mg/kg) | Route | Schedule | Collection^{c} | Collection^{d} | Collection^{c} |
| 14 | 3 | Vehicle | — | p.o. | Single | — | 6 h, n = 3 | — |
| | | | | | dose | | | |
| 15 | 12 | Compound | 10 | p.o. | Single | 1 h, 6 h, 12 h, | 1 h, 6 h, 12 h, | 6 h, 12 h; n = 3 |
| | | 133 | | | dose | 24 h; n = 3 | 24 h; n = 3 | |
| 16 | 12 | Compound | 10 | p.o. | Single | 1 h, 6 h, 12 h, | 1 h, 6 h, 12 h, | 6 h, 12 h; n = 3 |
| | | 144 | | | dose | 24h; n = 3 | 24h; n = 3 | |
| 17 | 12 | Compound | 10 | p.o. | Single | 1 h, 6 h, 12 h, | 1 h, 6 h, 12 h, | 6 h, 12 h; n = 3 |
| | | 198 | | | dose | 24h; n = 3 | 24h; n = 3 | |

Note:
^{a}N: animal number; Dosing volume: adjusted dosing volume based on body weight 10 μL/g.
^{b}The formulations for vehicle, Compound 133, Compound 144 and Compound 198 were 20% PEG400 + 80% (25% SBE-β-CD in ddH₂O).
^{c}Plasma and liver samples were collected at predetermined time points for PK analysis.
^{d}Tumor samples were collected at predetermined time points for PK and WB analysis.

TABLE 39
Study Design for the PK and PD Study Part III

| | | | | | | Time point | Time point | Time point |
|---|---|---|---|---|---|---|---|---|
| | | | Dose | Dosing | | for Plasma | for Tumor | for Liver |
| Group | N^{a} | Treatment^{b} | (mg/kg) | Route | Schedule | Collection^{c} | Collection^{d} | Collection^{c} |
| 18 | 3 | Vehicle | — | p.o. | Single | — | 6 h, n = 3 | — |
| | | | | | dose | | | |
| 19 | 12 | Compound | 10 | p.o. | Single | 1 h, 6 h, 12 | 1 h, 6 h, 12 | 6 h, 12 h; |
| | | 204 | | | dose | h, 24 h; n = 3 | h, 24 h; n = 3 | n = 3 |

Note:
^{a}N: animal number; Dosing volume: adjusted dosing volume based on body weight 10 μL/g.
^{b}The formulations for vehicle, Compound 204 were 20% PEG400 + 80% (25% SBE-β-CD in ddH₂O).
^{c}Plasma and liver samples were collected at predetermined time points for PK analysis.
^{d}Tumor samples were collected at predetermined time points for PK and WB analysis.

### Materials

### Animals and Housing Condition

One hundred and seventeen female BALB/c nude mice procured from Shanghai Lingchang Biological Science and Technology Co., LTD between the ages of six to eight weeks and with body weight between eighteen and twenty-two grams were used for the study. A spare mouse was also procured.

The mice were kept in individual ventilation cages at constant temperature and humidity with 3 or 6 animals in each cage. The cage was kept between approximately 20 to 26° C. with a humidity of 40-70%. The cages were made of polycarbonate with a size of 375 mm×215 mm×180 mm and corn cob bedding which was changed twice per week.

The animals were fed irradiation sterilized dry granule food during the entire study period. The animals had free access to food and sterile drinking water. Cages were identified with the following information: number of animals, sex, strain, date received, treatment, study number, group number and the starting date of the treatment and each animal was marked by ear coding.

Test Articles

| Compound | Molecular Weight | Formula Weight |
|---|---|---|
| Dabrafenib | 519.56 | 519.56 |
| Compound 157 | 954.02 | 954.02 |
| Compound 218 | 882.34 | 882.99 |
| Compound 144 | 954.02 | 954.02 |
| Compound 133 | 941.99 | 941.99 |
| Compound 198 | 924.04 | 924.04 |
| Compound 204 | 854.93 | 854.93 |

Reagents and Consumables

| No. | Reagents/Consumables | Vendor | Catalog # |
|---|---|---|---|
| 1 | RIPA Buffer | Sigma | R0278 |
| 2 | Phosphatase Inhibitor Cocktail 2 | Sigma | P5726 |
| 3 | Protease Inhibitor Cocktail | Roche | 04693124001 |
| 4 | Pierce ™ BCA Protein Assay Kit | Thermo | 23225 |
| | | Scientific | |
| 5 | NuPAGE ™ LDS Sample Buffer | Invitrogen | NP0007 |
| | (4×) | | |
| 6 | NuPAGE ™ Sample Reducing Agent | Invitrogen | NP0009 |
| | (10×) | | |
| 7 | NuPAGE ® MES SDS Running | Invitrogen | NP0002 |
| | Buffer (20×) | | |
| 8 | 20 × TBS | Bio-Serve | BS-P-15 |
| 9 | Tween 20 | Sigma | P2287 |
| 10 | Intercept ™ (TBS) Blocking Buffer | LI-COR | 927-60001 |
| 11 | PageRuler ™ Prestained Protein | Thermo | 26619 |
| | Ladder | Scientific | |
| 12 | iBlot ® 2 Transfer Stack Regular | Invitrogen | IB23001 |
| 13 | NuPAGE, Novex 4-12% Bis-Tris | Invitrogen | WG1403BOX |
| | Gel, 26 well | | |
| 14 | Bovine Serum Albumin | BBI life | A600332- |
| | | sciences | 0100 |
| 15 | SuperSignal ™ West Femto | Thermo | 34096 |
| | Maximum Sensitivity Substrate | Scientific | |
| 16 | Nonfat Milk | BD Difco | 232100 |
| 17 | SuperSignal West Pico | Thermo | 34078 |
| | Chemiluminescent Substrate | Scientific | |

1 × TBST: added 50 mL 20 × TBS to 949 mL H₂O, and then added 1 mL Tween 20, mixed well.
Running buffer: added 50 mL 20 × MES SDS Running Buffer to 950 mL H₂O, mixed well.

Instruments

| No. | Instruments | Vendor | Model # |
|---|---|---|---|
| 1 | Tissuelyser | Shanghai Jingxin | JXFSTPRP-CL |
| 2 | Electro- thermal incubator | Boxun | HPX-9162 MBE |
| 3 | SpectraMax | Molecular | M5e |
| | | Devices | |
| 4 | Dry baths | Hangzhou All | MK-10 |
| | | sheng Instruments | |
| | | Co., Ltd | |
| 5 | Xcell4 SureLock ® | Invitrogen | WR0100 |
| | Midi-cell | | |
| 6 | PowerPac ™ Basic Power | Bio-Rad | 1645050 |
| | Supply | | |
| 7 | Orbital Shaker | Qiling | TS-1 |
| 8 | iBlot ® 2 Gel Transfer | Invitrogen | iBlot 2 |
| | Device | | |
| 9 | Beads | Qiagen | 69989 |
| 10 | Amersham Imager 680 | GE Healthcare | AI680QC |
| 11 | Vortex Shaker | Kylin-Bell | Vortex-6 |
| 12 | Centrifuge | Eppendorf | 5424R |

Antibodies

| No. | Antibodies | Vendor | Catalog # |
|---|---|---|---|
| 1 | BRAF (D9T6S) antibody | Cell Signaling | CST-14814 |
| | | Technology | |
| 2 | phospho-p44/42 | Cell Signaling | CST-9101 |
| | MAPK (Erk1/2) | Technology | |
| | (Thr202/Tyr204) antibody | | |
| 3 | GAPDH antibody | Cell signal technology | CST-5174 |
| 4 | Goat Anti-Rabbit IgG H&L | Abcam | ab6721 |
| | (HRP) | | |

### Experimental Methods and Procedures

### Cell Culture

The A375 tumor cells were maintained in vitro in DMEM medium supplemented with 10 fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation. The details of reagents are shown in the Table below.

TABLE 40
Vendor and Cat No. of Cell Line and Reagents

| Cell Line/Reagent | Vendor | Catalog # |
|---|---|---|
| A375 | ATCC | CRL-1619 |
| DMEM | Gibco | 11995-065 |
| Penicillin-Streptomycin | Gibco | 15240-062 |
| 0.25% Trypsin-EDTA | Gibco | 25200-072 |
| FBS | Excell | FSP500 |
| DPBS | Corning | 21-031-CVC |
| Matrigel Matrix | Corning | 354234 |

### Tumor Inoculation

Each mouse was inoculated subcutaneously at the right flank with A375 cells (5×10⁶) in 0.2 mL of PBS supplemented with Matrigel (PBS: Matrigel=1:1) for tumor development. Animals were grouped for the treatment when the average tumor volume reached 243 mm³ for efficacy study part I and 413 mm³ for the PK and PD study part I on day 15, and 271 mm³ for efficacy part II and 443 mm³ for the PK and PD part II on day 18, and 463 mm³ for the PK and PD study part III on day 21. The test articles administration and the animal numbers in each group are shown in the above experimental tables.

### Reagents Used in the Study

TABLE 41
Vendor and Cat No. of Reagents Used for Formulation Preparation

| Reagent | Vendor | Catalog # |
|---|---|---|
| PEG400 | Sigma | P3265 |
| SBE-β-CD | Kunshan Ruisike Chemical Materials | 10059859 |
| HP-β-CD | Energy chemical | E080016 |

### Vehicle Preparation

### Preparation of 25% SBE-β-CD in ddH₂O

1) Accurately weighed 50 g of SBE-β-CD into a 500 mL glass bottle.
2) Added 150 mL volume of ddH₂O, stirred to obtain a clear solution.
3) Added ddH₂O to 200 mL and mixed well.

### Preparation of 11.1% HP-β-CD in ddH₂O

1. Accurately weighed 22.2 g of HP-β-CD into a 500 mL glass bottle.
2. Added 150 mL volume of ddH₂O, stirred to obtain a clear solution.
3. Added ddH₂O to 200 mL and mixed well.

### Testing Article Formulation Preparation

TABLE 42
Formulation of Dabrafenib Preparation

| | | | | Volume of | Volume of | |
|---|---|---|---|---|---|---|
| | | | | PEG400 to | 11.1% HP-β- | |
| | | Conc. of | Compound | be added | CD to be | Dosing |
| Compound | Dose | solution | amount | (mL) | added (mL) | Solution |
| ID | (mg/kg) | (mg/mL) | (mg) | (10%) | (90%) | observations |
| Dabrafenib^{a} | 100 | 10 | 68.27 | 0.682 | 6.138 | Suspension |

Note:
^{a}The formulation was prepared twice a week.

TABLE 43
Formulations of C4T's Compounds Preparation

| | | | | Volume of | Volume of | |
|---|---|---|---|---|---|---|
| | | | | PEG400 to | 25% SBE- | |
| | | Conc. of | Compound | be added | β-CD to be | Dosing |
| Compound^{a} | Dose | solution | amount | (mL) | added (mL) | Solution |
| ID | (mg/kg) | (mg/mL) | (mg) | (20%) | (80%) | observations |
| Compound 218 | 10 | 1 | 6.73 | 1.311 | 5.245 | Solution |
| Compound 157 | 10 | 1 | 6.60 | 1.303 | 5.210 | Solution |
| Compound 133 | 10 | 1 | 7.18 | 1.410 | 5.638 | Solution |
| Compound 144 | 10 | 1 | 6.87 | 1.347 | 5.388 | Solution |
| Compound 198 | 10 | 1 | 6.38 | 1.262 | 5.046 | Solution |
| Compound 204 | 10 | 1 | 3.84 | 0.761 | 3.044 | Solution |

Note:
^{a}The formulations were prepared once every two days.

### Observations

At the time of routine monitoring, the animals were checked daily for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset. Animals that were observed to be in a continuing deteriorating condition or their tumor size exceeding 3000 mm³ were euthanized prior to death or before reaching a comatose state.

### Tumor Measurements and the Endpoints

Prior to the onset of drug treatment, mice were measured for tumor size in two dimensions using a caliper, and the tumor volume (mm³) was calculated using formula V=0.5 a×b² where a and b are the long and short diameters of the tumor in mm, respectively. Mice were randomized into different treatment groups based on the tumor volume.

The major endpoint was to see if the tumor growth could be delayed or mice could be cured. The tumor size was then used for calculations of T/C value. The T/C value (in percent) is an indication of antitumor effectiveness; T and C are the mean volumes of the treated and control groups, respectively on specified day.

TGI was calculated for each group using the formula: TGI (%)=[1−(Tᵢ−T₀)/(Cᵢ−C₀)]×100; Tᵢ is the average tumor volume of a treatment group on a given day, To is the average tumor volume of the treatment group on the day of treatment start, Cᵢ is the average tumor volume of the vehicle control group on the same day with Tᵢ, and C₀ is the average tumor volume of the vehicle group on the day of treatment start.

### Experimental Endpoint and Sample Collection

### Plasma Sample Preparation

The plasma samples were collected at predetermined time points for PK analysis. The mice were euthanized at predetermined time points post the treatment. The blood samples were collected into the 1.5 mL Eppendorf tubes containing 15 μL of 0.5 M EDTA-K2. And then the anticoagulant blood samples were centrifuged at 2,500 g at 4° C. for 15 minutes. The plasma samples were separated into 2 aliquots. The 1^{st} was used for PK analysis, and the 2^{nd} served as backup. The samples were stored at −80° C. before bioanalysis.

### Tumor Sample Preparation

The tumor samples were collected at predetermined time points for PK and WB analysis. The tumor was dissected out and split into 3 parts. The 1^{st} part was used for PK analysis, the 2^{nd} part was used for WB analysis and the last one served as backup for study. The samples were transferred into pre-labeled tubes and quickly froze in liquid nitrogen within 2 minutes after dissection. The samples were stored at −80° C. before bioanalysis.

### Liver Sample Preparation

Liver samples were collected at predetermined time points for PK analysis. The left lobe of the liver was dissected out and split into 2 parts. The 1^{st} part was used for PK analysis and the 2^{nd} part served as the backup for the study. The samples were transferred into pre-labeled tubes and quickly froze in liquid nitrogen within 2 minutes after dissection. The samples were stored at −80° C. before bioanalysis.

### Pharmacokinetics Assay

### Pharmacokinetics Assay for Efficacy Study

The harvested plasma and liver samples were processed for pharmacokinetics analysis by LC-MS/MS with a concentration range of 1.00-1,000 ng/mL for Dabrafenib in plasma and liver homogenate, 10.0-10,000 ng/mL for Compound 218, Compound 157, Compound 133, Compound 144 and Compound 198 in plasma and liver homogenate.

### Pharmacokinetics Assay for the PK and PD Study

The harvested plasma, tumor and liver samples were processed for pharmacokinetics analysis by LC-MS/MS with a concentration range of 5.00-10,000 ng/mL for Compound 218, Compound 157 and Compound 133 in plasma, 0.500-1,000 ng/ml for Compound 218 in liver and tumor homogenate, 0.200-400 ng/mL for Compound 157, Dabrafenib and Compound 133 in liver and tumor homogenate, 0.500-1000 ng/mL for Dabrafenib and Compound 204 in plasma, 0.200-200 ng/mL for Compound 204 in liver and tumor homogenate, 20.0-20,000 ng/mL for Compound 144 and Compound 198 in plasma, 1.00-1,000 ng/mL for Compound 144 and Compound 198 in liver and tumor homogenate.

### Western Blotting Assay

### Protein Extraction & Protein Quantification

The previously snap-frozen cells were removed from the −80° C. freezer and then cut up to about 30-100 mg of tissue per sample. The tissues were then placed in 2 mL microcentrifuge tube and 400 μL of RIPA Buffer was then added. The RIPA Buffer contained 1% Protease Inhibitor Cocktail and 1% Phosphatase Inhibitor Cocktail 2.

The tumors were then ground with a freeze grinder at 50 Hz for five minutes and then the tissue lysates were kept on ice for thirty minutes. The lysates were centrifuged at 15,000 rpm at 4° C. for ten minutes and then the supernatants were gently transferred to a new microcentrifuge tube kept on ice.

The protein concentration was measured with a Pierce™ BCA Protein Assay Kit. Then the samples were diluted, according to the result of BCA protein quantification, to the same final concentration (at 2 μg/μL) using RIPA buffer plus 4× LDS Sample Buffer and 10× Sample Reducing Agent. The samples were then heated at 100° C. for ten minutes. The denatured samples were then used for Western blotting or kept in the −80° C. refrigerator for long term storage.

### Western Blotting

The protein samples were loaded to NuPAGE® Novex 4-12% Bis-Tris gel with 10 μL per slot. The electrophoresis with MES running buffer was ran at 80 V for 30 minutes and then at 120 V for 90 minutes. Then protein was transferred to NC (nitrocellulose) membranes with iBlot®2 Gel Transfer Device, P3 for 7 minutes. Afterwards, the membranes were cut, the interest membranes parts were kept and washed with 5-10 mL 1× TBST three times for 5 minutes each. Then the non-specific protein was blocked by 5-10 mL 1× TBST with 5% nonfat milk at room temperature for 1 hour. The membrane was then washed with 5-10 mL 1× TBST three times for 5 minutes each. The membrane was then incubated with 5-10 mL diluted primary antibody (BRAF antibody was diluted at a ratio of 1:1000, phospho-p44/42 MAPK antibody was diluted at a ratio of 1:2000 and GAPDH antibody was diluted at a ratio of 1:2000) in 1× TBST with 5% BSA at 4° C. with gentle agitation overnight. Next, the membrane was washed with 5-10 mL 1× TBST three times for 10 minutes each. The resulting membrane was incubated with 5-10 mL HRP-conjugated secondary antibody (generally 1:3000) with gentle agitation for 1 hour at room temperature. Then the membrane was washed with 5-10 mL 1× TBST three times for 10 minutes each. Afterwards, the HRP substrate from West Femto Maximum Sensitivity kit was added to the NC membranes and chemiluminescence was detected by AI680QC.

### Quantification of Band Intensity

Intensity of individual bands were quantified using Image Studio Lite Ver 5.2 software.

### Statistical Analysis

Band intensity of individual bands was obtained for analysis. Protein expression was quantitated in relation to a reference protein, and then the normalized folds of protein expression in each sample were ratios to vehicle group.

The normalized data were interpreted by GraphPad Prism. Statistical analysis of difference in the protein expression of interests among the groups was conducted.

### Data Management and Statistical Analyses

A one-way ANOVA followed by Dunnett's multiple comparison test was performed to compare tumor volume among vehicle group and treatment groups. All data were analyzed using GraphPad Prism, **** indicates p<0.0001.

### Results

### Results for Efficacy Study

### Results for Efficacy Study Part I

### Body Weight Change

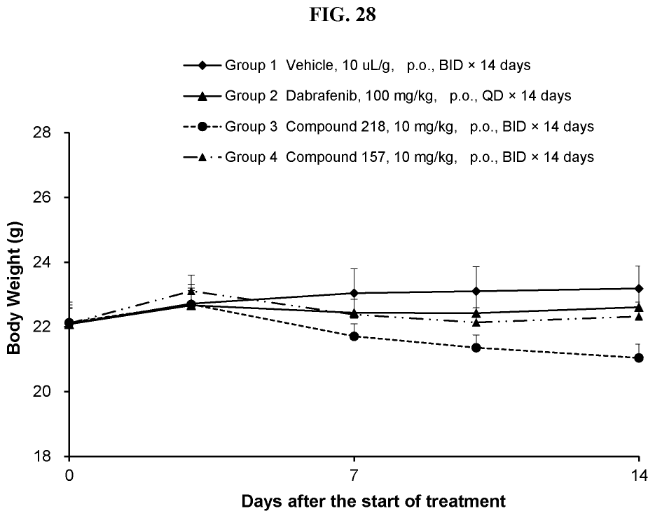

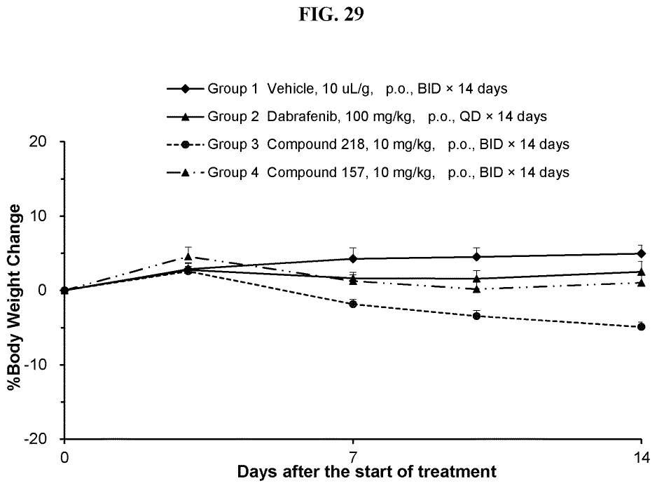

Animal body weight was monitored regularly as an indirect measurement of toxicity. Body weight changes after administration of vehicle, dabrafenib, Compound 218, and Compound 157 to female BALB/c nude mice bearing A375 Xenografts are shown in FIG. 28 and FIG. 29.

### Tumor Growth Curve

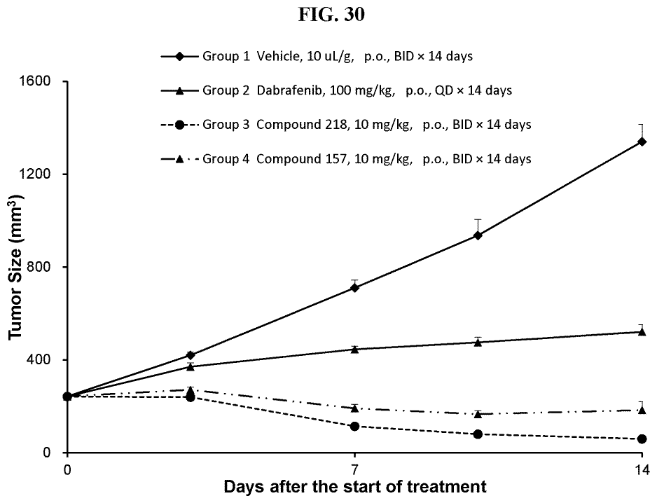

Tumor growth curves after administration of vehicle, dabrafenib, Compound 218, and Compound 157 to Female BALB/c nude Mice Bearing A375 Xenografts are shown in FIG. 30.

### Tumor Volume Trace

Mean tumor size over time in female BALB/c nude mice bearing A375 human melanoma xenografts is shown in Table 44.

TABLE 44
Tumor Volume Trace Over Time (mm³)^{a}

| | Days | | | | |
|---|---|---|---|---|---|
| Group# | 0 | 3 | 7 | 10 | 14 |
| Group 1, Vehicle, | 243 ± 3 | 420 ± 14 | 710 ± 34 | 936 ± 69 | 1340 ± 74 |
| p.o., BID × 14 days | | | | | |
| Group 2, Dabrafenib, 100 mg/kg | 243 ± 4 | 370 ± 17 | 445 ± 13 | 475 ± 23 | 521 ± 31 |
| p.o., QD × 14 days | | | | | |
| Group 3, Compound 218, 10 mg/kg, | 243 ± 5 | 240 ± 21 | 114 ± 9 | 80 ± 9 | 60 ± 8 |
| p.o., BID × 14 days | | | | | |
| Group 4, Compound 157, 10 mg/kg, | 243 ± 4 | 272 ± 12 | 191 ± 17 | 166 ± 15 | 183 ± 37 |
| p.o., BID × 14 days | | | | | |

Note:
Data are shown as Mean ± SEM.

### Tumor Growth Inhibition Analysis

Tumor growth inhibition of Dabrafenib, Compound 218 and Compound 157 in the treatment of female BALB/c nude mice bearing A375 xenografts was calculated based on tumor volume measured on PG-D14. TGI and T/C were calculated respectively, and the data are detailed in the Table 45.

TABLE 45
Tumor Growth Inhibition Analysis (T/C and TGI)^{a}

| | Tumor Size | | | |
|---|---|---|---|---|
| | (mm³)^{a} | T/C^{b} | TGI^{c} | p |
| Treatment | on PG-D14 | (%) | (%) | value^{d} |
| Group 1, Vehicle, | 1340 ± 74 | / | / | / |
| p.o., BID × 14 days | | | | |
| Group 2, Dabrafenib, | 521 ± 31 | 38.87 | 74.65 | **** |
| 100 mg/kg, p.o., QD × 14 days | | | | |
| Group 3, Compound 218, | 60 ± 8 | 4.47 | 116.68 | **** |
| 10 mg/kg, p.o., BID × 14 days | | | | |
| Group 4, Compound 157, | 183 ± 37 | 13.68 | 105.48 | **** |
| 10 mg/kg, p.o., BID × 14 days | | | | |

Note:
^{a}Data are shown as Mean ± SEM.
^{b}Antitumor activity (T/C) was determined by dividing the average tumor volume for treated group (T) by the average tumor volume for control group (C).
^{c}Tumor Growth Inhibition (TGI) was calculated using the formula: TGI (%) = (1 − (T − T₀)/(C − C₀))*100, where T = mean tumor size at the terminal end of treatment group; T₀ = mean tumor size at day 0 of treatment group; C = mean tumor size at the terminal end of control group; and C₀ = mean tumor size at day 0 of control group.
^{d}A one-way ANOVA followed by Dunnett's multiple comparison test was performed to compare tumor volume among vehicle group and treatment groups. All data were analyzed using GraphPad Prism, **** indicates p < 0.0001.

### Pharmacokinetics Results

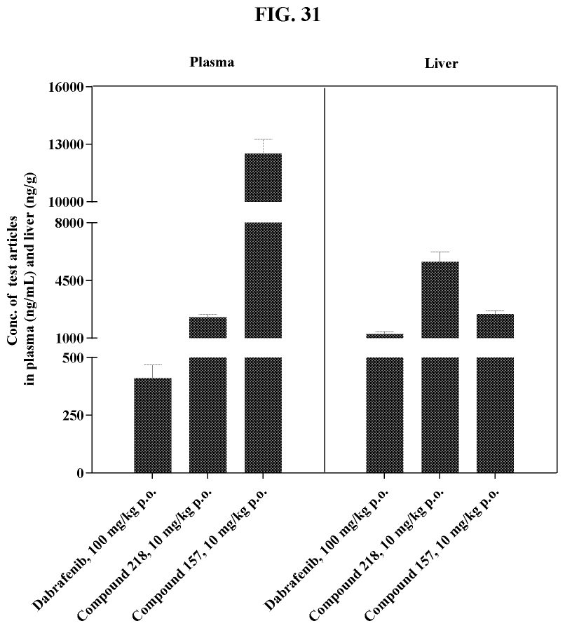

The concentration of dabrafenib, Compound 218, and Compound 157 in plasma and liver samples collected 6 hours following last treatment of female BALB/c nude mice bearing A375 tumors are shown in Table 46 and FIG. 31.

TABLE 46
Mean Concentration of Test Articles in Plasma and Liver

| | | Concen- | Concen- |
|---|---|---|---|
| | | tration | tration |
| | | in Plasma | in Liver |
| | Sample | (ng/mL) ± | (ng/g) ± |
| Group^{a} | time | SEM | SEM |
| Group 2, Dabrafenib, | 6 h | 410 ± 58.2 | 1242 ± 162 |
| 100 mg/kg, p.o., QD × 14 days | | | |
| Group 3, Compound 218, | 6 h | 2267 ± 201 | 5637 ± 588 |
| 10 mg/kg, p.o., BID × 14 days | | | |
| Group 4, Compound 157, | 6 h | 12517 ± 745 | 2448 ± 217 |
| 10 mg/kg, p.o., BID × 14 days | | | |

### Results for Efficacy Study Part II

### Body Weight Change

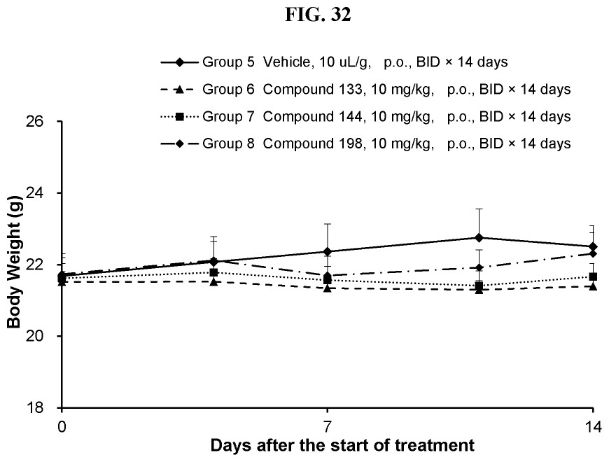

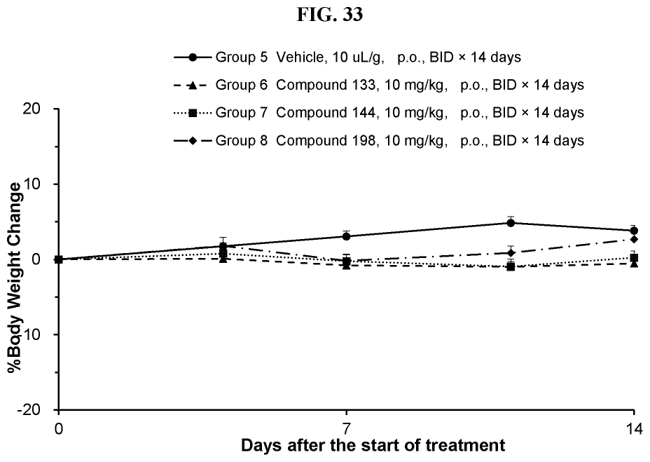

Animal body weight was monitored regularly as an indirect measurement of toxicity. Body weight changes after administration of vehicle, Compound 133, Compound 144, Compound 198 to female BALB/c nude mice bearing A375 xenografts are shown in FIG. 32 and FIG. 33.

### Tumor Growth Curve

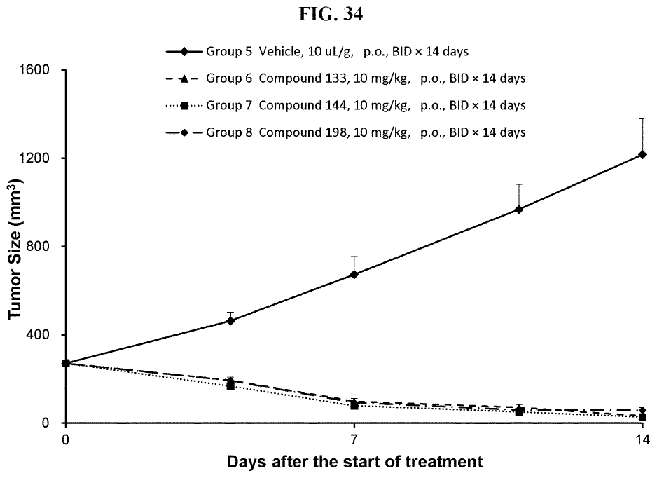

Tumor growth curves after administration of vehicle, Compound 133, Compound 144, Compound 198 to Female BALB/c nude Mice Bearing A375 Xenografts. are shown in FIG. 34.

### Tumor Volume Trace

Mean tumor size over time in female BALB/c nude mice bearing A375 human melanoma xenografts is shown in Table 47.

TABLE 47
Tumor Volume Trace Over Time (mm³)^{a}

| | Days | | | | |
|---|---|---|---|---|---|
| Group# | 0 | 4 | 7 | 11 | 14 |
| Group 5, Vehicle, | 271 ± | 464 ± | 673 ± | 968 ± | 1216 ± |
| p.o., BID × 14 days | 15 | 39 | 82 | 113 | 162 |
| Group 6, Compound 133, | 271 ± | 193 ± | 99 ± | 71 ± | 32 ± |
| 10 mg/kg, p.o., BID × 14 days | 18 | 15 | 13 | 13 | 7 |
| Group 7, Compound 144, | 271 ± | 167 ± | 79 ± | 51 ± | 28 ± |
| 10 mg/kg, p.o., BID × 14 days | 16 | 10 | 11 | 6 | 5 |
| Group 8, Compound 198, | 271 ± | 192 ± | 93 ± | 60 ± | 58 ± |
| 10 mg/kg, p.o., BID × 14 days | 15 | 16 | 12 | 8 | 13 |

Note:
^{a}Data are shown as Mean ± SEM.

### Tumor Growth Inhibition Analysis

Tumor growth inhibition of Compound 133, Compound 144 and Compound 198 in the treatment of female BALB/c nude mice bearing A375 xenografts was calculated based on tumor volume measured on PG-D14. TGI and T/C were calculated respectively, and the data are detailed in the Table 48.

TABLE 48
Tumor Growth Inhibition Analysis (T/C and TGI)

| | Tumor Size | | | |
|---|---|---|---|---|
| | (mm³)^{a} | T/C^{b} | TGI^{c} | p |
| Treatment | on PG-D14 | (%) | (%) | value^{d} |
| Group 5, Vehicle, | 1216 ± 162 | / | / | / |
| p.o., BID × 14 days | | | | |
| Group 6, Compound 133, | 32 ± 7 | 2.65 | 125.20 | **** |
| 10 mg/kg, p.o., BID × 14 days | | | | |
| Group 7, Compound 144, | 28 ± 5 | 2.27 | 125.70 | **** |
| 10 mg/kg, p.o., BID × 14 days | | | | |
| Group 8, Compound 198, | 58 ± 13 | 4.79 | 122.45 | **** |
| 10 mg/kg, p.o., BID × 14 days | | | | |

Note:
^{a}Data are shown as Mean ± SEM.
^{b}Antitumor activity (T/C) was determined by dividing the average tumor volume for treated group (T) by the average tumor volume for control group (C).
^{c}Tumor Growth Inhibition (TGI) was calculated using the formula: TGI (%) = (1 − (T − T₀)/(C − C₀))*100, where T = mean tumor size at the terminal end of treatment group; T₀ = mean tumor size at day 0 of treatment group; C = mean tumor size at the terminal end of control group; and C₀ = mean tumor size at day 0 of control group.
^{d}A one-way ANOVA followed by Dunnett's multiple comparison test was performed to compare tumor volume among vehicle group and treatment groups. All data were analyzed using GraphPad Prism, **** indicates p < 0.0001.

### Pharmacokinetics Results

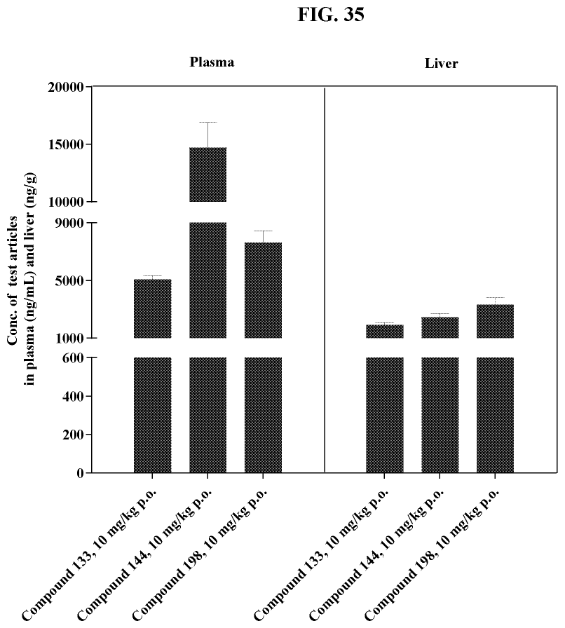

The concentration of Compound 133, Compound 144, Compound 198 in plasma and liver samples collected 6 hours following treatment of female BALB/c nude mice bearing A375 tumors are shown in Table 49 and FIG. 35.

TABLE 49
Mean Concentration of Test Articles in Plasma and Liver

| | | Concen- | Concen- |
|---|---|---|---|
| | | tration | tration |
| | | in Plasma | in Liver |
| | Sample | (ng/mL) ± | (ng/g) ± |
| Group | time | SEM | SEM |
| Group 6, Compound 133, | 6 h | 5070 ± 270 | 1907 ± 169 |
| 10 mg/kg, p.o., BID × 14 days | | | |
| Group 7, Compound 144, | 6 h | 14715 ± 2224 | 2468 ± 252 |
| 10 mg/kg, p.o., BID × 14 days | | | |
| Group 8, Compound 198, | 6 h | 7645 ± 799 | 3325 ± 479 |
| 10 mg/kg, p.o., BID × 14 days | | | |

### Results for the PK and PD Study

### Results for the PK and PD Study Part I

### Pharmacokinetics Results

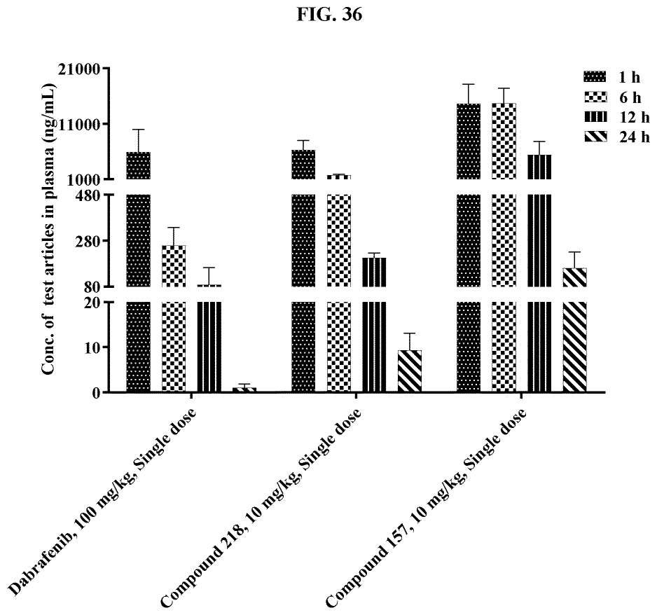

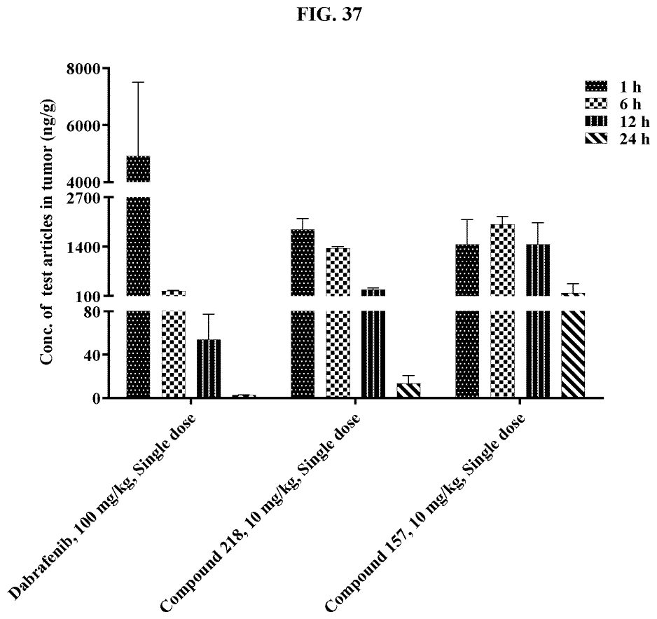

The concentration of dabrafenib, Compound 218, and Compound 157 in the treatment of female BALB/c nude mice bearing A375 xenografts are shown in Table 50 and FIG. 36-38.

TABLE 50
Mean Concentration of Test Articles in Plasma, Tumor and Liver

| | | | Concen- | Concen- | Concen- |
|---|---|---|---|---|---|
| | | | tration | tration | tration |
| | | | in Plasma | in Tumor | in Liver |
| | Sample | | (ng/mL) ± | (ng/g) ± | (ng/g) ± |
| Group | time | | SEM | SEM | SEM |
| Group 11, | 1 | h | 5917 ± 2378 | 4920 ± 1492 | — |
| Dabrafenib, | 6 | h | 259 ± 45.4 | 237 ± 4.91 | 1009 ± 111 |
| 100 mg/kg, | 12 | h | 88.7 ± 42.7 | 54.0 ± 13.5 | 277 ± 125 |
| p.o., Single | 24 | h | 1.09 ± 0.467 | 1.87 ± 0.9 | — |
| dose | | | | | |
| Group 12, | 1 | h | 6307 ± 978 | 1853 ± 164 | — |
| Compound | 6 | h | 1750 ± 96.1 | 1363 ± 21.9 | 5713 ± 300 |
| 218, 10 mg/kg, | 12 | h | 206 ± 11.5 | 268 ± 26.7 | 972 ± 40.9 |
| p.o., Single | 24 | h | 6.20 ± 3.47 | 13.4 ± 4.31 | — |
| dose | | | | | |
| Group 13, | 1 | h | 14667 ± 2002 | 1463 ± 376 | — |
| Compound | 6 | h | 14700 ± 1563 | 1990 ± 120 | 2003 ± 132 |
| 157, 10 mg/kg, | 12 | h | 5420 ± 1398 | 1467 ± 329 | 1319 ± 233 |
| p.o., Single | 24 | h | 161 ± 40.1 | 183 ± 137 | — |
| dose | | | | | |

### Western Blotting Analysis Results

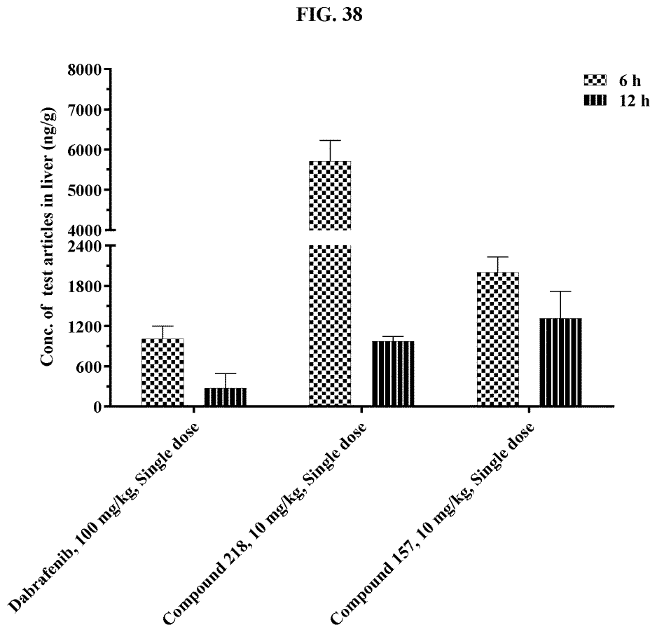

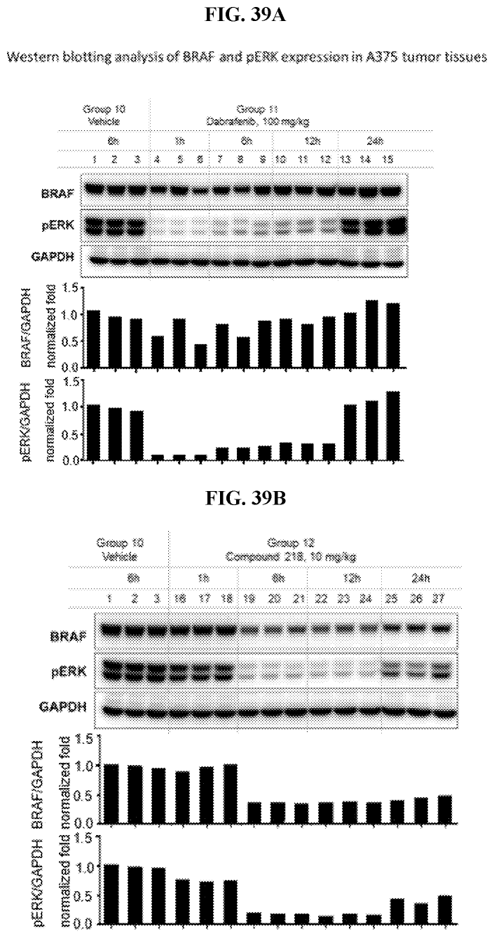

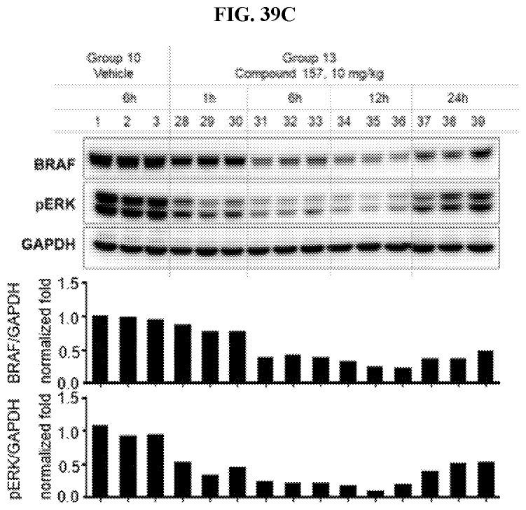

The results of BRAF and p-ERK expression level in A375 tumor samples after administration of vehicle, dabrafenib, Compound 218, and Compound 157 are shown in FIG. 39.

### Results for the PK and PD Study Part II

### Pharmacokinetics Results

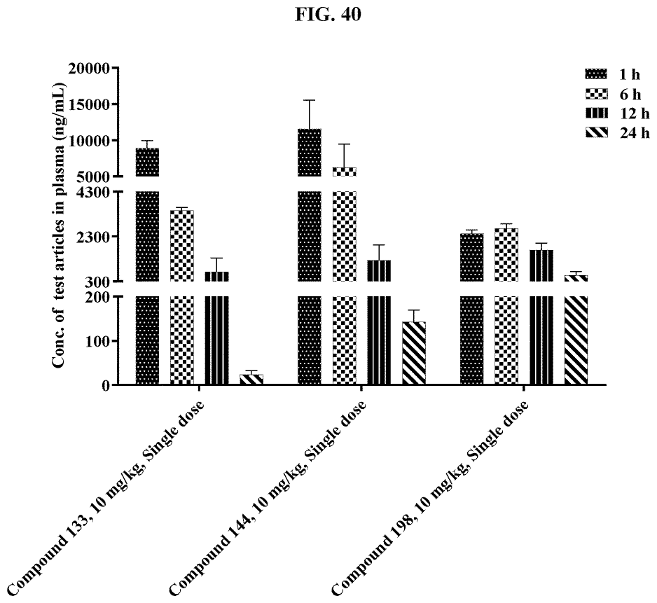

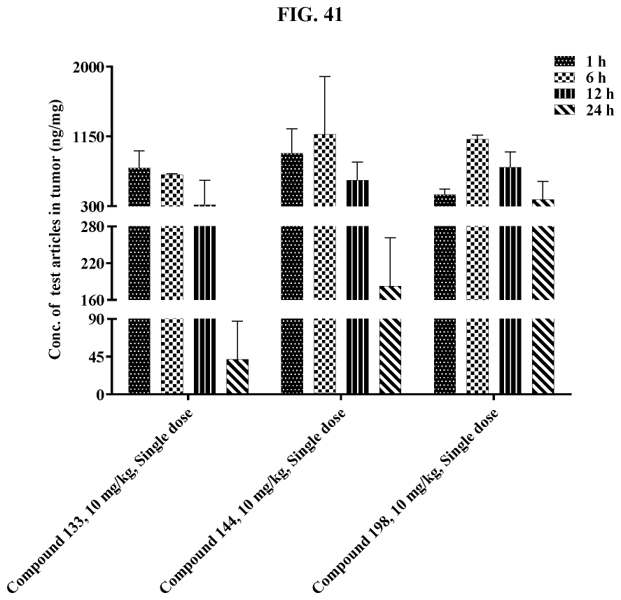

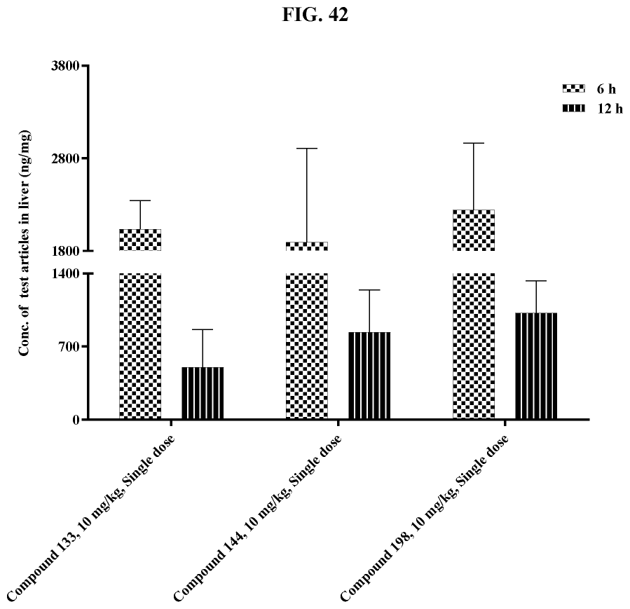

The concentration of Compound 133, Compound 144, and Compound 198 in the treatment of female BALB/c nude mice bearing A375 xenografts are shown in Table 51 and FIG. 40-42.

TABLE 51
Mean Concentration of Test Articles in Plasma, Tumor and Liver

| | | | Concen- | Concen- | Concen- |
|---|---|---|---|---|---|
| | | | tration | tration | tration |
| | | | in Plasma | in Tumor | in Liver |
| | Sample | | (ng/mL) ± | (ng/g) ± | (ng/g) ± |
| Group | time | | SEM | SEM | SEM |
| Group 15, | 1 | h | 8880 ± 604 | 764 ± 122 | — |
| Compound 133, | 6 | h | 3463 ± 69.8 | 686 ± 6.23 | 2033 ± 179 |
| 10 mg/kg, | 12 | h | 733 ± 348 | 316 ± 172 | 503 ± 207 |
| p.o., Single dose | 24 | h | 23.4 ± 5.56 | 41.7 ± 26.4 | — |
| Group 16, | 1 | h | 11607 ± 2265 | 946 ± 172 | — |
| Compound 144, | 6 | h | 6220 ± 1882 | 1178 ± 406 | 1894 ± 584 |
| 10 mg/kg, | 12 | h | 1244 ± 395 | 616 ± 127 | 873 ± 234 |
| p.o., Single dose | 24 | h | 143 ± 15.4 | 183 ± 45.4 | — |
| Group 17, | 1 | h | 2430 ± 91.7 | 441 ± 38.8 | — |
| Compound 198, | 6 | h | 2667 ± 117 | 1117 ± 28.5 | 2243 ± 416 |
| 10 mg/kg, | 12 | h | 1703 ± 172 | 776 ± 107 | 1023 ± 177 |
| p.o., Single dose | 24 | h | 573 ± 94.2 | 383 ± 128 | — |

### Western Blotting Analysis Results

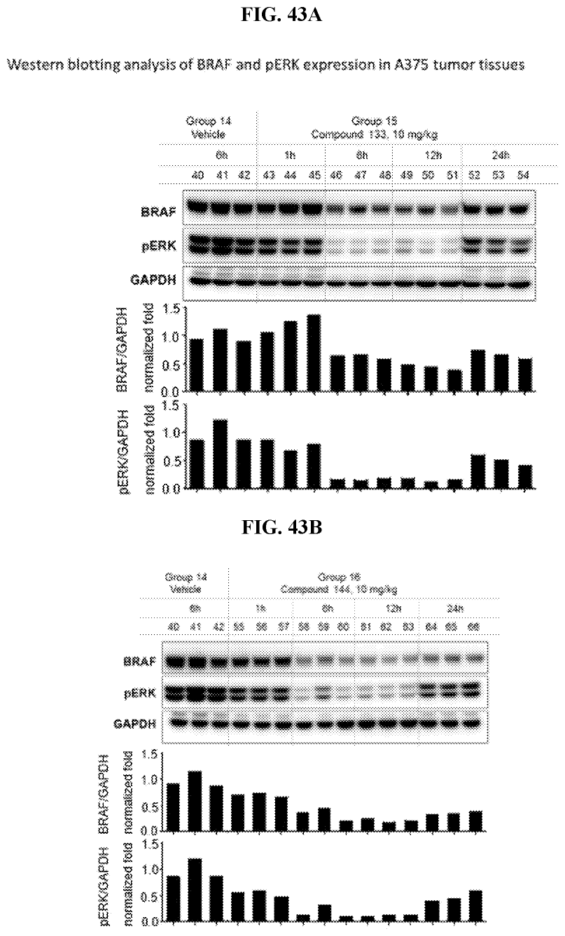

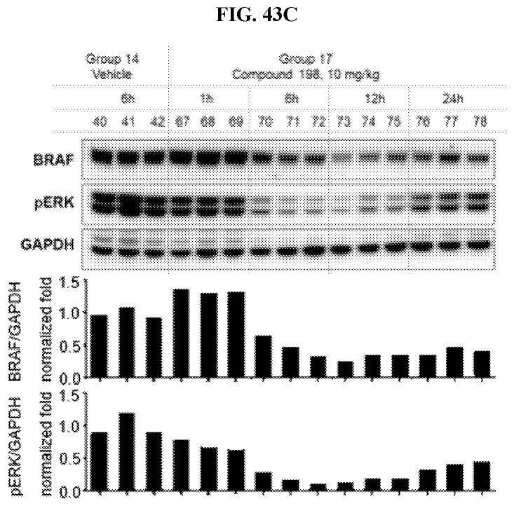

The results of BRAF and p-ERK expression level in A375 tumor samples after administration of vehicle, Compound 133, Compound 144, and Compound 198. are shown in FIG. 43.

### Results for PKPD Study Part III

### Pharmacokinetics Results

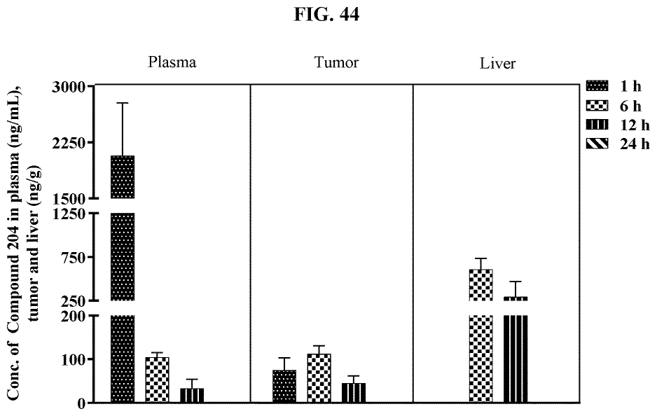

The concentration of Compound 204 in the treatment of female BALB/c nude mice bearing A375 xenografts are shown in Table 52 and FIG. 44.

TABLE 52
Mean Concentration of Compound
204 in Plasma, Tumor and Liver

| | | | Concen- | Concen- | Concen- |
|---|---|---|---|---|---|
| | | | tration | tration | tration |
| | | | in Plasma | in Tumor | in Liver |
| | Sample | | (ng/mL) ± | (ng/g) ± | (ng/g) ± |
| Group | time | | SEM | SEM | SEM |
| Group 19, | 1 | h | 2067 ± 407 | 74.7 ± 16.5 | — |
| Compound 204, | 6 | h | 104 ± 6.18 | 112 ± 11.0 | 606 ± 73.4 |
| 10 mg/kg, | 12 | h | 32.9 ± 12.1 | 45.0 ± 9.93 | 291 ± 103 |
| p.o., Single dose | 24 | h | ND | ND | — |

Note:
ND: Not determined.

### Western Blotting Analysis Results

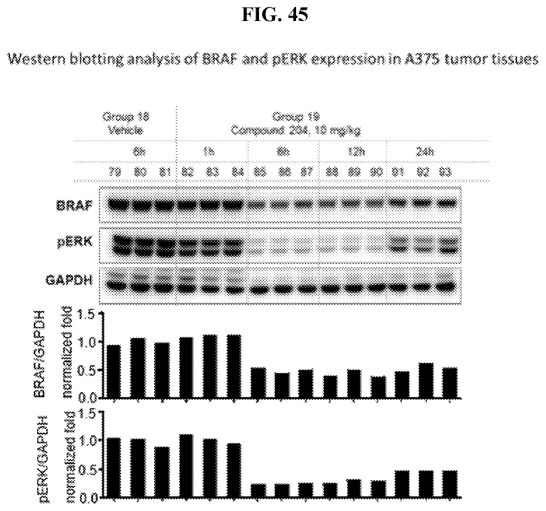

The results of BRAF and p-ERK expression level in A375 tumor samples after administration of vehicle and Compound 204 are shown in FIG. 45.

### Example 10 BSEP Vesicular Transport Assay

The vesicular transport (VT) assays were performed using inside out membrane vesicles derived from HEK293 cells expressing the specified human bile salt export pump (BSEP) efflux transporter. The membrane vesicles were procured from SOLVO Biotechnology Charles River Laboratories, Szeged, Hungary.

The principle of the assay relies on measuring the accumulation of substrate into inside out vesicles containing the efflux transporter. By utilizing inside out vesicles, the ATP-dependent transport of substrate is directed into the vesicular lumen, where low permeability compounds are then essentially trapped.

For the IC₅₀ screen inhibition assay, 10 concentrations of each compound were tested in the presence of ATP (active transport) in 1 replicate.

For the 1 concentration inhibition assay, each compound was tested in the presence of ATP (active transport) in 2 replicates.

The solvent control (DMSO) was tested in the presence of ATP (active transport) and in the presence of AMP (background) in 8 replicates.

### Evaluation of Compounds as an Inhibitor of Human BSEP in the Vesicular Transport Inhibition Assay (IC₅₀ Determination)

Vesicular transport inhibition assays were performed using the specific parameters for each transporter inhibition assay described in Table 53.

TABLE 53
Experimental conditions for vesicular transporter assays

| Transporter | Probe | Fold Acceptance |
|---|---|---|
| (host cell line) | substrate | criteria |
| human BSEP (HEK293) | TC (0.2 μM) | ≥15 |

TC: Taurocholate

Table 54 specifies treatment groups and concentration of compounds, that were examined in the vesicular transport inhibition assay. Detailed data of IC₅₀ screen inhibition assay are shown in Table 55 and detailed data of 1 concentration inhibition assay are shown in Table 56.

The vesicular transport inhibition assay was performed with 4 mM Mg-ATP. For the IC₅₀ screen inhibition assay, 10 concentrations of each compound were tested in the presence of ATP in 1 replicate. For the 1 concentration inhibition assay, 1 concentration of each compound was tested in the presence of ATP (active transport) in 2 replicates. The solvent control (DMSO) was tested in the presence of 4 mM Mg-ATP (active transport) and in the presence of 4 mM AMP (background) in 8 replicates.

TABLE 54
Vesicular transport inhibition assay conditions and controls
Transporters: BSEP

| Assay Conditions | Controls | Readout |
|---|---|---|
| 4 mM Mg-ATP | 4 mM Mg-ATP or 4 | ATP-dependent probe |
| 1 concentration screen: | mM AMP | substrate transport (cpm): |
| compounds at 1 | Functionality control: | in the presence and |
| concentration, No. of | Solvent control | absence of compound |
| replicates: 2 | No. of replicates: 8 | % inhibition of transport |
| IC₅₀ screen: | | by compound |
| compounds at 10 | | IC₅₀ (μM, if |
| concentration, No. of | | applicable) |
| replicates: 1 | | |

### Vesicular Transport Inhibition Assay and Samples

### Assay Plate

The BSEP membrane vesicle suspension was mixed with the ice-cold transport buffer, then the taurocholate probe substrate solution was added (Reaction mixture, 50 μL per well). The compounds were added to the reaction mixture at 1.5-times the final assay concentration. predetermined time points.

### Pre-Warming Step (Assay Plate)

Reaction mixtures and the start reagent (Mg-ATP or AMP in transport buffer) were pre-warmed separately for 15 minutes to reach the incubation temperature of 37° C. Reaction mixtures and the Start reagent (Mg-ATP or AMP in transport buffer) were pre-warmed separately for 15 minutes to reach the incubation temperature of 37° C.

### Incubation (Assay Plate)

The reaction was initiated by the addition of the start reagent (25 μL) to the reaction mixture (diluting the reaction mixture to the final concentration). The organic solvent content of the final reaction mixture did not exceed 1.5% (v/v). After the 5-minute incubation at 37° C., the reaction was stopped by the addition of 250 μL ice-cold washing mix to each well. The reaction was initiated by the addition of the start reagent (25 μL) to the reaction mixture (diluting the reaction mixture to the final concentration). The organic solvent content of the final reaction mixture did not exceed 1.5% (v/v). After the 5-minute incubation at 37° C., the reaction was stopped by the addition of 250 μL ice-cold washing mix to each well.

### Filter Plate

The samples were then be transferred to a filter plate and washed with 200 μL cold washing mix5-times to remove any free compounds or taurocholate probe substrate. The amount of taurocholate probe substrate inside the filtered vesicles were determined by liquid scintillation counting. The samples were then be transferred to a filter plate and washed (5-times with 200 μL cold washing mix) to remove any free compounds or taurocholate probe substrate. The amount of taurocholate probe substrate inside the filtered vesicles were determined by liquid scintillation counting.

TABLE 55
Vesicular transport inhibition assay (IC₅₀ determination)

| Compound ID | BSEP Inhibition/IC₅₀ |
|---|---|
| Compound 26 | 21770 |
| Compound 28 | 1959 |
| Compound 30 | 8520 |
| Compound 31 | 4232 |
| Compound 33 | 10060 |
| Compound 38 | 963.6 |
| Compound 39 | 1556 |
| Compound 54 | 11010 |
| Compound 60 | 7883 |
| Compound 70 | 2084 |
| Compound 83 | 14100 |
| Compound 93 | >40000 |
| Compound 118 | >40000 |
| Compound 133 | 6219 |
| Compound 141 | >40000 |
| Compound 144 | 7941 |
| Compound 145 | 21630 |
| Compound 156 | >40000 |
| Compound 157 | 8833 |
| Compound 158 | 7531 |
| Compound 160 | 1584 |
| Compound 177 | 7363 |
| Compound 180 | 12180 |
| Compound 85 | 9814 |
| Compound 86 | 9883 |
| Compound 198 | 15250 |
| Compound 204 | >40000 |
| Compound 218 | 13210 |
| Compound 246 | 8785 |
| Compound 250 | 8446 |
| Compound 254 | 1744 |
| Compound 259 | 12760 |
| Compound 260 | 12720 |
| Compound 261 | >40000 |
| Compound 269 | >40000 |
| Compound 273 | 20580 |
| Compound 277 | 11860 |
| Compound 280 | 26550 |

TABLE 56
One concentration inhibition assay

| Compound ID | BSEP Inhibition SD Inhibition % |
|---|---|
| Compound 6 | 27 |
| Compound 7 | 30 |
| Compound 9 | 52 |
| Compound 8 | 38 |
| Compound 10 | 29 |
| Compound 11 | 35 |
| Compound 12 | 59 |
| Compound 13 | 40 |
| Compound 15 | 59 |
| Compound 16 | 65 |
| Compound 17 | 42 |
| Compound 21 | 31 |
| Compound 22 | 54 |
| Compound 24 | 42 |
| Compound 25 | 57 |
| Compound 26 | 56 |
| Compound 27 | 69 |
| Compound 28 | 81 |
| Compound 30 | 52 |
| Compound 31 | 70 |
| Compound 32 | 46 |
| Compound 33 | 55 |
| Compound 3 | 49 |
| Compound 4 | 45 |
| Compound 35 | 48 |
| Compound 38 | 90 |
| Compound 39 | 89 |
| Compound 40 | 69 |
| Compound 41 | 51 |
| Compound 43 | 29 |
| Compound 45 | 42 |
| Compound 46 | 60 |
| Compound 47 | 56 |
| Compound 48 | 70 |
| Compound 50 | 31 |
| Compound 51 | 33 |
| Compound 52 | 44 |
| Compound 53 | 52 |
| Compound 54 | 61 |
| Compound 56 | 37 |
| Compound 57 | 49 |
| Compound 58 | 73 |
| Compound 59 | 49 |
| Compound 60 | 70 |
| Compound 61 | 43 |
| Compound 62 | 68 |
| Compound 63 | 74 |
| Compound 64 | 49 |
| Compound 65 | 28 |
| Compound 67 | 85 |
| Compound 68 | 51 |
| Compound 69 | 61 |
| Compound 70 | 80 |
| Compound 71 | 63 |
| Compound 73 | 35 |
| Compound 76 | 41 |
| Compound 78 | 38 |
| Compound 79 | 22 |
| Compound 80 | 27 |
| Compound 81 | 21 |
| Compound 82 | 45 |
| Compound 83 | 50 |
| Compound 84 | 73 |
| Compound 87 | 46 |
| Compound 90 | 39 |
| Compound 92 | 39 |
| Compound 93 | 34 |
| Compound 94 | 55 |
| Compound 95 | 31 |
| Compound 96 | 50 |
| Compound 98 | 46 |
| Compound 100 | 53 |
| Compound 101 | 38 |
| Compound 102 | 45 |
| Compound 103 | 27 |
| Compound 104 | 57 |
| Compound 106 | 48 |
| Compound 109 | 44 |
| Compound 110 | 48 |
| Compound 113 | 44 |
| Compound 115 | 47 |
| Compound 116 | 23 |
| Compound 117 | 42 |
| Compound 118 | 27 |
| Compound 119 | 60 |
| Compound 121 | 32 |
| Compound 122 | 4 |
| Compound 123 | 29 |
| Compound 124 | 31 |
| Compound 126 | 32 |
| Compound 128 | 52 |
| Compound 129 | 64 |
| Compound 130 | 41 |
| Compound 131 | 53 |
| Compound 132 | 58 |
| Compound 133 | 42 |
| Compound 134 | 45 |
| Compound 136 | 20 |
| Compound 137 | 42 |
| Compound 138 | 48 |
| Compound 140 | 36 |
| Compound 141 | 19 |
| Compound 143 | 26 |
| Compound 144 | 42 |
| Compound 145 | 40 |
| Compound 146 | 70 |
| Compound 147 | 38 |
| Compound 149 | 70 |
| Compound 151 | 13 |
| Compound 153 | 44 |
| Compound 155 | 55 |
| Compound 156 | 24 |
| Compound 157 | 39 |
| Compound 158 | 58 |
| Compound 160 | 84 |
| Compound 161 | 67 |
| Compound 163 | 77 |
| Compound 164 | 22 |
| Compound 165 | 68 |
| Compound 166 | 30 |
| Compound 167 | 18 |
| Compound 168 | 62 |
| Compound 170 | 73 |
| Compound 172 | 85 |
| Compound 174 | 58 |
| Compound 176 | 57 |
| Compound 177 | 48 |
| Compound 178 | 18 |
| Compound 179 | 19 |
| Compound 180 | 45 |
| Compound 181 | 58 |
| Compound 182 | 31 |
| Compound 183 | 53 |
| Compound 184 | 25 |
| Compound 185 | 49 |
| Compound 186 | 39 |
| Compound 187 | 38 |
| Compound 188 | 39 |
| Compound 189 | 89 |
| Compound 190 | 89 |
| Compound 191 | 47 |
| Compound 192 | 58 |
| Compound 193 | 42 |
| Compound 194 | 62 |
| Compound 195 | 44 |
| Compound 196 | 77 |
| Compound 85 | 57 |
| Compound 86 | 50 |
| Compound 197 | 29 |
| Compound 198 | 36 |
| Compound 199 | 24 |
| Compound 200 | 38 |
| Compound 201 | 68 |
| Compound 203 | 37 |
| Compound 204 | 26 |
| Compound 205 | 25 |
| Compound 206 | 46 |
| Compound 209 | 55 |
| Compound 212 | 94 |
| Compound 214 | 90 |
| Compound 208 | 57 |
| Compound 216 | 57 |
| Compound 217 | 88 |
| Compound 218 | 44 |
| Compound 219 | 65 |
| Compound 220 | 44 |
| Compound 221 | 38 |
| Compound 222 | 58 |
| Compound 223 | 66 |
| Compound 224 | 91 |
| Compound 225 | 89 |
| Compound 226 | 28 |
| Compound 227 | 90 |
| Compound 228 | 45 |
| Compound 229 | 74 |
| Compound 230 | 47 |
| Compound 231 | 59 |
| Compound 232 | 72 |
| Compound 233 | 85 |
| Compound 234 | 51 |
| Compound 235 | 75 |
| Compound 236 | 71 |
| Compound 237 | 69 |
| Compound 238 | 60 |
| Compound 240 | 74 |
| Compound 241 | 51 |
| Compound 242 | 49 |
| Compound 243 | 73 |
| Compound 245 | 60 |
| Compound 246 | 35 |
| Compound 247 | 40 |
| Compound 248 | 31 |
| Compound 249 | 71 |
| Compound 250 | 51 |
| Compound 251 | 34 |

### Example 11 HT29 Cell Viability Assay (CTGlo)

The effect of compounds of the invention on cell viability was assessed based on the quantification of ATP using the CellTiter-Glo® 2.0 (referred to here as CTGlo) Cell Viability Assay (Catalog No. G9243, Promega, Madison, WI, USA), which signals the presence of metabolically active cells. The HT29 human colorectal adenocarcinoma (CRC) cell line with epithelial morphology, was used in these assays. This cell line carries the oncogenic alterations BRAF V600E, PIK3CA P449T, and TP53 R273H.

HT29 cells were obtained from ATCC (Catalog No. HTB-38, Manassas, VA, USA) (also referred to here as HT29.1) and were maintained in McCoy's 5a Medium Modified, (Catalog No. 30-2007, ATCC, Manassas, VA, USA) supplemented with 10% fetal bovine serum (FBS; Catalog No. 16000044, Gibco, Grand Island, NY, USA) at 37° C. in an atmosphere of 5% CO₂ in air. The cells were routinely sub-cultured to maintain cell density between 3×10⁵- 1.5×10⁶ cells/mL. Cells were washed with phosphate buffered saline, pH 7.4 (PBS; Catalog No. 10010049, ThermoFisher Scientific, Waltham, MA, USA), trypsinized with Trypsin-EDTA (0.25%), phenol red (Catalog No. 25200056, ThermoFisher Scientific, Waltham, MA, USA) for 5 min at 37° C., and resuspended in growth media. An aliquot was diluted 2× with a 0.4% Trypan Blue solution (Catalog No. 15250061, ThermoFisher Scientific, Waltham, MA, USA) and cell count was determined. Cell concentration was adjusted with growth media to 2.0×10⁴ cells/mL.

To determine the effect of compounds on cell viability, 50 μL HT29 cells suspended in growth media at 2.0×10⁴ cells/mL (for a cell density of 1000 cells/well) were dispensed to each well of a 384-well, black TC-treated microplates (Catalog No. 3571, Corning, Glendale, CA, USA).

Test compounds were prepared by dissolving neat compounds in dimethyl sulfoxide (DMSO; Catalog No. D8418, Sigma-Aldrich, Inc., St. Louis, MO, USA) to generate 10 mM stock solution and stored at −20° C. The 10 mM DMSO stock solutions were serially diluted (half log titration) in DMSO to generate a 10-point dose series with a final concentration of 10 μM, in duplicate. Using an Echo 550 Acoustic Liquid Handler (Beckman Coulter Life Sciences, Indianapolis, IN, USA), plated cells were treated with 50 nL of serially diluted test compound solutions in duplicate. 50 nL DMSO was transferred to control wells. Plates were incubated at 37° C., 5% CO₂ for 72 hours. CellTiter-Glo® reagent was then added to the cells and luminescence signals were measured after 4 hours using an EnVision™ Multimode plate reader (Perkin Elmer, Santa Clara, CA).

For data normalization, cells untreated with test compounds at Time=72 hours were set to 100% (equivalent to maximal cell growth after 96 hours). Media-only wells were set to −100% (equivalent to cytotoxicity). Percent Viability was determined by normalizing the signal with positive and DMSO treated negative controls on the same microtiter plate. The half-maximal inhibition of cell growth (GI50) was computed from where the fitted curve crosses 50%. The Emax, or maximum effect of each compound, represents the lowest % viability achieved following compound treatment.

FIG. 46 shows HT29 percent viability over concentration when test compounds were compound 26 and compound 141. The experiment was conducted at least once for each compound, where if the experiment was conducted multiple times, then the data shown is the average of the two or more experiments.

### Example 12 Efficacy, Pharmacokinetics, and Pharmacodynamics of Compound 26 and 141 in a Model of CNS Activity

For the efficacy study, agents were tested in A375 human melanoma cancer cell derived xenografts (CDX). This cell line was isolated from the primary melanoma of a 54-year-old female and is known to have a homozygous BRAF V600E mutation. The A375 tumor cells were maintained in vitro in DMEM medium supplemented with 10% fetal bovine serum and 1% Penicillin-Streptomycin at 37° C. in an atmosphere of 5% CO₂ in air. The tumor cells were routinely subcultured twice weekly. The cells growing in an exponential growth phase were harvested and counted for tumor inoculation.

For this CNS study, A375 cells with a luciferase tag for tracking tumor cell signal, were injected intracranially into 6-8-week-old female BALB/c nude mice (Zhejiang Vital River Laboratory Animal Co., LTD. Beijing, China). The mice were anaesthetized, restrained, and sterilized prior to surgery. A sagittal cut was made above the anterior parietal and occipital bone with sterilized scalpel, the skull was unrevealed and cleaned with cotton bud, in this case the anterior fontanel could be available. A 2 mm hole was created to the right of the anterior fontanel and 0.5 mm ahead of the coronal on the skull with a cranial drill. Cell suspensions in 20% Matrigel were mixed and aspirated into injector carefully to avoid bubbles and wiped to avoid extracranial tumor genesis. The needle was inserted vertically into the cranial hole and the cell mixture was slowly injected for 2 minutes and held for another minute to decrease the drainage of the cell suspension. The wound was closed, and a subcutaneous injection of 5 mg/kg Meloxicam was given to relieve the pain and continued for 3 days. The mice were monitored closely until they woke up after the surgery. During the study, the care and use of animals was conducted in accordance with the regulations of the Association for Assessment and Accreditation of Laboratory Animal Care (AAALAC). After inoculation, the animals were checked daily for morbidity and mortality. At the time of routine monitoring, the animals would be checked for any effects of tumor growth and treatments on normal behavior such as mobility, food and water consumption, body weight gain/loss, eye/hair matting, and any other abnormal effect.

The administration of the test agents began three days after inoculation. CNS tumor-bearing mice were randomly assigned to groups of 8 mice and administered Compound 26 at 10 mg/kg orally (PO) twice a day (BID), Compound 141 at 10 mg/kg PO/BID, encorafenib at 35 mg/kg once a day orally, (PO/QD), or the vehicle alone PO/BID. Measurements for the presence of the A375 luciferase (CRL-1619-LUC2, ATCC, Manassas, VA, USA) cells within the CNS were done with IVIS Lumina III machine. Images were collected twice weekly to follow the growth of the models. The tumor-bearing mice were weighed and intraperitoneally administered luciferin (Perkin Elmer Inc-122799) at a dosage of 150 mg/kg. After 10 min, mice were pre-anesthetized with the mixture gas of oxygen and isoflurane. The bioluminescence value at 15 min post luciferin injection was recorded as the final value and plotted. The endpoint for individual mice was reached when the mouse body weight decreased by 20% when compared to their starting weight.

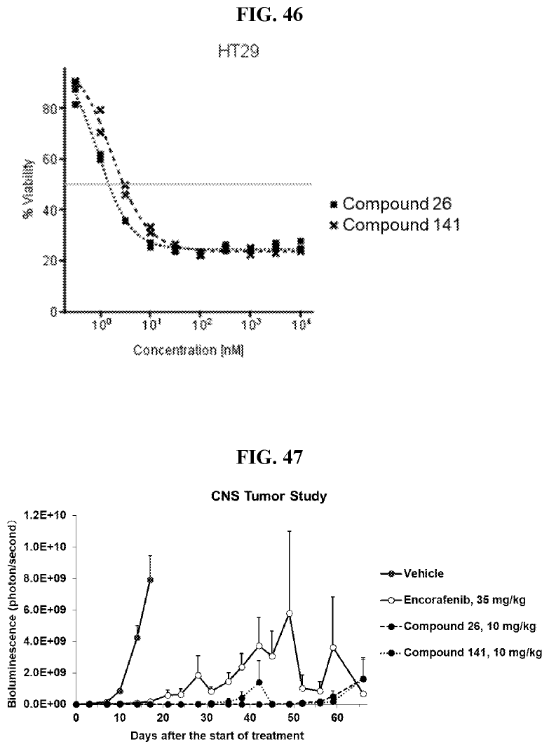

The mice in the vehicle control group all reached their endpoint within the first three weeks of study with a median survival of 18 days. Encorafenib prolonged the survival to a median of 46 days. As of day 75, two mice in the Compound 26 group and 2 mice in the Compound 141 group have demonstrated end point cut offs and were terminated. The luciferase signal increased concordantly with the increase in symptoms of the CNS tumors. The luciferase signal for the mice removed from study were not carried forward, therefore a sudden drop in luciferase signal over time is due to mice with a heavy tumor burden being terminated and their high luciferase signal not being carried forward. The compounds were tolerated and none of the groups showed body weight loss prior to an increase in luciferase signal. The change in luciferase signal over time, representing tumor burden, is shown in FIG. 47. The survival curve for the mice on study is shown in FIG. 48. As of day 75, both Compound 26 and Compound 141 treated mice have a 75% survival rate. The dosing was stopped on day 120 and the survival curve for the mice on study up to 200 days is shown in FIG. 85. The median survival of mice dosed with Compound 26 is 91 days and the median survival of mice dosed with Compound 141 is 89.5 days.

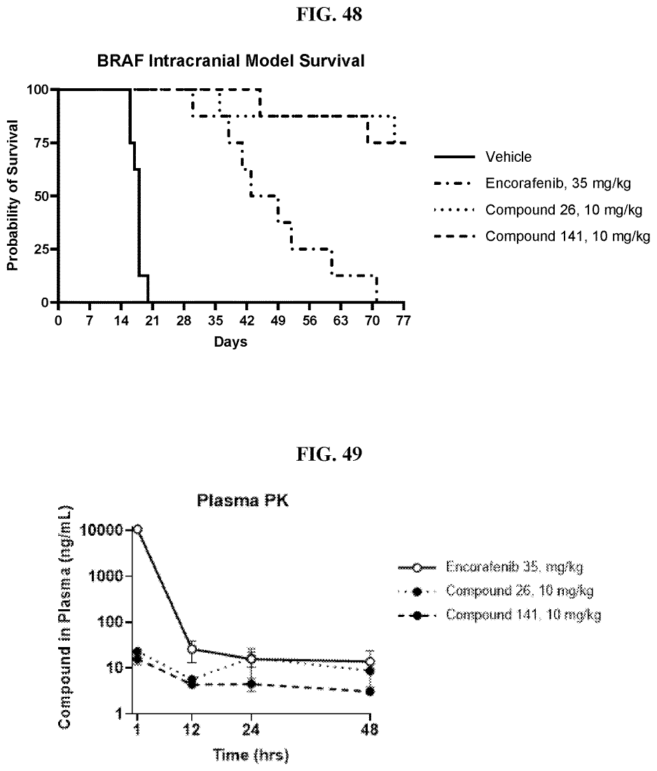

To determine the level of CNS and plasma exposure of the test articles compared to the standard of care, a separate pharmacokinetics/pharmacodynamics (PK/PD) study was conducted. Mice were inoculated with luciferase tagged A375 cells as stated above. The tumors were left to establish and expand for 14 days before the study. Mice were randomized and treated with Compound 26 at 10 mg/kg orally (PO), Compound 141 at 10 mg/kg PO, encorafenib at 35 mg/kg, or the vehicle alone. Samples from 3 mice per treatment group were collected at the following timepoints, 1, 12, 24, and 48 hours. To test the plasma samples, the mice were anesthetized, and blood was collected into a tube with EDTA-K2, centrifuged, transferred to a fresh tube, and stored frozen. Following blood collection, the mice were perfused for 15 minutes, decapitated, and the brain was collected and frozen. The harvested plasma and brain were processed for pharmacokinetics analysis by LC/MS/MS with a concentration range of 1-1,000 ng/mL and pharmacodynamics analysis by Western Blot. The concentrations of Compound 26, 141, and encorafenib found in the plasma are shown in FIG. 49, and the concentration found in the brain is shown in FIG. 50. The pharmacodynamics of mutant BRAF protein degradation in the inoculated CNS tumor 1, 12, 24, 48 hours post treatment was quantified and shown in FIG. 51.

### Example 13 In vivo PKPD study of Compound 26 and Compound 141

### Method

Agents were administered to BALB/c nude mice bearing A375 human melanoma xenografts. Dabrafenib was dosed via p.o. once on day 1, Compound 26 and Compound 141 were dosed via p.o. from day 0 to day 1. Dabrafenib was dosed at 100 mg/kg (QD). Compound 26 was dosed at 1.5 mg/kg (BID), 2 mg/kg (TID), 3 mg/kg (BID), 6 mg/kg (QD) and 10 mg/kg (BID). Compound 26 was dosed at 0.75 mg/kg (BID), 1 mg/kg (TID), 1.5 mg/kg (BID), 3 mg/kg (QD) and 5 mg/kg (BID). This study was non-GLP.

### Experimental Design

TABLE 57
Study Design for PKPD Study

| | | | | | | Timepoint | Timepoint |
|---|---|---|---|---|---|---|---|
| | | | Dose | Dosing | | for Plasma | for Tissues |
| Group | N^{a} | Treatment^{b} | (mg/kg) | Route | Schedule | Collection^{c} | Collection^{d} |
| 13 | 3 | Vehicle | / | p.o. | BID × 1 | — | 10 h |
| | | | | | day | | |
| 14 | 3 | Dabrafenib | 100 | p.o. | QD × 1 | 0.25 h, 1 h | 1 h |
| | 3 | | | | day | 0.5 h, 4 h | 4 h |
| | 3 | | | | | 2 h, 10 h | 10 h |
| | 3 | | | | | 8 h, 24 h | 24 h |
| | 3 | | | | | 36 h, 48 h | 48 h |
| 15 | 3 | Compound | 1.5 | p.o. | BID × 1 | 0.25, 8 h post 1^{st} | 1 h post 2^{nd} |
| | | 26 | | | day | dose; | dose |
| | | | | | | 0.25 h, 1 h post 2^{nd} | |
| | | | | | | dose | |
| | 3 | | | | | 0.5, 12 h post 1^{st} | 4 h post 2^{nd} |
| | | | | | | dose; | dose |
| | | | | | | 0.5 h, 4 h post 2^{nd} | |
| | | | | | | dose | |
| | 3 | | | | | 1 h post 1^{st} dose; | 10 h post 2^{nd} |
| | | | | | | 2 h, 10 h post 2^{nd} | dose |
| | | | | | | dose | |
| | 3 | | | | | 2 h post 1^{st} dose; | 24 h post 2^{nd} |
| | | | | | | 24 h post 2^{nd} dose | dose |
| | 3 | | | | | 4 h post 1^{st} dose; | 48 h post 2^{nd} |
| | | | | | | 36 h, 48 h post 2^{nd} | dose |
| | | | | | | dose | |
| 16 | 3 | Compound | 2 | p.o. | TID × 1 | 0.25, 8 h post 1^{st} | 1 h post 3^{rd} |
| | | 26 | | | day | dose; | dose |
| | | | | | | 0.25 h, 1 h post 3^{rd} | |
| | | | | | | dose | |
| | 3 | | | | | 0.5 h post 1^{st} dose; | 4 h post 3^{rd} |
| | | | | | | 0.25 h post 2^{nd} dose; | dose |
| | | | | | | 0.5 h, 4 h post 3^{rd} | |
| | | | | | | dose | |
| | 3 | | | | | 1 h post 1^{st} dose; | 10 h post 3^{rd} |
| | | | | | | 2 h post 2^{nd} dose; | dose |
| | | | | | | 2 h, 10 h post 3^{rd} | |
| | | | | | | dose | |
| | 3 | | | | | 2 h post 1^{st} dose; | 24 h post 3^{rd} |
| | | | | | | 8 h post 2^{nd} dose; | dose |
| | | | | | | 24 h post 3^{rd} dose | |
| | 3 | | | | | 4 h post 1^{st} dose; | 48 h post 3^{rd} |
| | | | | | | 36 h, 48 h post 3^{rd} | dose |
| | | | | | | dose | |
| 17 | 3 | Compound | 3 | p.o. | BID × 1 | 0.25, 8 h post 1^{st} | 0.25 h post 2^{nd} |
| | | 26 | | | day | dose; | dose |
| | | | | | | 0.25 h post 2^{nd} dose | |
| | 3 | | | | | 0.5, 12 h post 1 st | 0.5 h post 2^{nd} |
| | | | | | | dose; | dose |
| | | | | | | 0.5 h post 2^{nd} dose | |
| | 3 | | | | | 1 h post 1^{st} dose; | 1 h post 2^{nd} |
| | | | | | | 1 h post 2^{nd} dose | dose |
| | 3 | | | | | 2 h post 1^{st} dose; | 2 h post 2^{nd} |
| | | | | | | 2 h post 2^{nd} dose | dose |
| | 3 | | | | | 4 h post 1^{st} dose; | 4 h post 2^{nd} |
| | | | | | | 4 h post 2^{nd} dose | dose |
| | 3 | | | | | 10 h post 2^{nd} dose | 10 h post 2^{nd} |
| | | | | | | | dose |
| | 3 | | | | | 24 h post 2^{nd} dose | 24 h post 2^{nd} |
| | | | | | | | dose |
| | 3 | | | | | 36 h post 2^{nd} dose | 36 h post 2^{nd} |
| | | | | | | | dose |
| | 3 | | | | | 48 h post 2^{nd} dose | 48 h post 2^{nd} |
| | | | | | | | dose |
| 18 | 3 | Compound | 6 | p.o. | QD × 1 | 0.25 h, 1 h | 1 h |
| | 3 | 26 | | | day | 0.5 h, 4 h | 4 h |
| | 3 | | | | | 2 h, 10 h | 10 h |
| | 3 | | | | | 8 h, 24 h | 24 h |
| | 3 | | | | | 36 h, 48 h | 48 h |
| 19 | 3 | Compound | 10 | p.o. | BID × 1 | 0.25, 8 h post 1^{st} | 1 h post 2^{nd} |
| | | 26 | | | day | dose; | dose |
| | | | | | | 0.25 h, 1 h post 2^{nd} | |
| | | | | | | dose | |
| | 3 | | | | | 0.5, 12 h post 1^{st} | 4 h post 2^{nd} |
| | | | | | | dose; | dose |
| | | | | | | 0.5 h, 4 h post 2^{nd} | |
| | | | | | | dose | |
| | 3 | | | | | 1 h post 1^{st} dose; | 10 h post 2^{nd} |
| | | | | | | 2 h, 10 h post 2^{nd} | dose |
| | | | | | | dose | |
| | 3 | | | | | 2 h post 1^{st} dose; | 24 h post 2^{nd} |
| | | | | | | 24 h post 2^{nd} dose | dose |
| | 3 | | | | | 4 h post 1^{st} dose; 36 | 48 h post 2^{nd} |
| | | | | | | h, 48 h post 2^{nd} dose | dose |
| 20 | 3 | Compound | 0.75 | p.o. | BID × 1 | 0.25, 8 h post 1^{st} | 1 h post 2^{nd} |
| | | 141 | | | day | dose; | dose |
| | | | | | | 0.25 h, 1 h post 2^{nd} | |
| | | | | | | dose | |
| | 3 | | | | | 0.5, 12 h post 1^{st} | 4 h post 2^{nd} |
| | | | | | | dose; | dose |
| | | | | | | 0.5 h, 4 h post 2^{nd} | |
| | | | | | | dose | |
| | 3 | | | | | 1 h post 1^{st} dose; | 10 h post 2^{nd} |
| | | | | | | 2 h, 10 h post 2^{nd} | dose |
| | | | | | | dose | |
| | 3 | | | | | 2 h post 1^{st} dose; | 24 h post 2^{nd} |
| | | | | | | 24 h post 2^{nd} dose | dose |
| | 3 | | | | | 4 h post 1^{st} dose; | 48 h post 2^{nd} |
| | | | | | | 36 h, 48 h post 2^{nd} | dose |
| | | | | | | dose | |
| 21 | 3 | Compound | 1 | p.o. | TID × 1 | 0.25, 8 h post 1^{st} | 1 h post 3^{rd} |
| | | 141 | | | day | dose; | dose |
| | | | | | | 0.25 h, 1 h post 3^{rd} | |
| | | | | | | dose | |
| | 3 | | | | | 0.5 h post 1^{st} dose; | 4 h post 3^{rd} |
| | | | | | | 0.25 h post 2^{nd} dose; | dose |
| | | | | | | 0.5 h, 4 h post 3^{rd} | |
| | | | | | | dose | |
| | 3 | | | | | 1 h post 1^{st} dose; | 10 h post 3^{rd} |
| | | | | | | 2 h post 2^{nd} dose; | dose |
| | | | | | | 2 h, 10 h post 3^{rd} | |
| | | | | | | dose | |
| | 3 | | | | | 2 h post 1^{st} dose; | 24 h post 3^{rd} |
| | | | | | | 8 h post 2^{nd} dose; | dose |
| | | | | | | 24 h post 3^{rd} dose | |
| | 3 | | | | | 4 h post 1^{st} dose; | 48 h post 3^{rd} |
| | | | | | | 36 h, 48 h post 3^{rd} | dose |
| | | | | | | dose | |
| 22 | 3 | Compound | 1.5 | p.o. | BID × 1 | 0.25, 8 h post 1^{st} | 0.25 h post 2^{nd} |
| | | 141 | | | day | dose; | dose |
| | | | | | | 0.25 h post 2^{nd} dose | |
| | 3 | | | | | 0.5, 12 h post 1^{st} | 0.5 h post 2^{nd} |
| | | | | | | dose; | dose |
| | | | | | | 0.5 h post 2^{nd} dose | |
| | 3 | | | | | 1 h post 1^{st} dose; | 1 h post 2^{nd} |
| | | | | | | 1 h post 2^{nd} dose | dose |
| | 3 | | | | | 2 h post 1^{st} dose; | 2 h post 2^{nd} |
| | | | | | | 2 h post 2^{nd} dose | dose |
| | 3 | | | | | 4 h post 1^{st} dose; | 4 h post 2^{nd} |
| | | | | | | 4 h post 2^{nd} dose | dose |
| | 3 | | | | | 10 h post 2^{nd} dose | 10 h post 2^{nd} |
| | | | | | | | dose |
| | 3 | | | | | 24 h post 2^{nd} dose | 24 h post 2^{nd} |
| | | | | | | | dose |
| | 3 | | | | | 36 h post 2^{nd} dose | 36 h post 2^{nd} |
| | | | | | | | dose |
| | 3 | | | | | 48 h post 2^{nd} dose | 48 h post 2^{nd} |
| | | | | | | | dose |
| 23 | 3 | Compound | 3 | p.o. | QD × 1 | 0.25 h, 1 h | 1 h |
| | 3 | 141 | | | day | 0.5 h, 4 h | 4 h |
| | 3 | | | | | 2 h, 10 h | 10 h |
| | 3 | | | | | 8 h, 24 h | 24 h |
| | 3 | | | | | 36 h, 48 h | 48 h |
| 24 | 3 | Compound | 5 | p.o. | BID × 1 | 0.25, 8 h post 1^{st} | 1 h post 2^{nd} |
| | | 141 | | | day | dose; | dose |
| | | | | | | 0.25 h, 1 h post 2^{nd} | |
| | | | | | | dose | |
| | 3 | | | | | 0.5, 12 h post 1^{st} | 4 h post 2^{nd} |
| | | | | | | dose; | dose |
| | | | | | | 0.5 h, 4 h post 2^{nd} | |
| | | | | | | dose | |
| | 3 | | | | | 1 h post 1^{st} dose; | 10 h post 2^{nd} |
| | | | | | | 2 h, 10 h post 2^{nd} | dose |
| | | | | | | dose | |
| | 3 | | | | | 2 h post 1^{st} dose; | 24 h post 2^{nd} |
| | | | | | | 24 h post 2^{nd} dose | dose |
| | 3 | | | | | 4 h post 1^{st} dose; | 48 h post 2^{nd} |
| | | | | | | 36 h, 48 h post 2^{nd} | dose |
| | | | | | | dose | |

Note:
^{a}N: animal number; Dosing volume: adjusted dosing volume based on body weight 10 μL/g.
^{b}The formulations for vehicle, Compound 26 and Compound 141 were 20% PEG400 + 80% (25% SBE-β-CD in ddH₂O). The formulation for Dabrafenib was 10% PEG400 + 90% (11.1% HP-β-CD in ddH₂O).
^{c}Plasma samples were collected at predicted time for PK analysis.
^{d}Tumor, brain, liver, lung, spleen and skin samples were collected at predicted time for PK and
WB analysis.

### Materials

### Animals and Housing Condition

One hundred and ninety-two female BALB/c nude Mus Musculus mice procured from Shanghai Lingchang Biological Science and Technology Co., LTD between the ages of six to eight weeks and with body weight between eighteen and twenty-two grams were used for the study. A spare mouse was also procured.

The mice were kept in individual ventilation cages at constant temperature and humidity with 3 animals in each cage. The cage was kept between approximately 20 to 26° C. with a humidity of 0.40-70%. The cages were made of polycarbonate with a size of 375 mm×215 mm×180 mm and corn cob bedding was changed twice per week.

The animals were fed irradiation sterilized dry granule food during the entire study period. The animals had free access to food and sterile drinking water. Cages were identified with the following information: number of animals, gender, strain, date received, treatment, study number, group number and the starting date of the treatment and each animal was marked by ear coding.

Test Articles

| Compound | Molecular Weight | Formula Weight |
|---|---|---|
| Dabrafenib | 519.56 | 519.56 |
| Compound 26 | 911.33 | 911.99 |
| Compound 141 | 895.34 | 895.99 |

### Reagents and Materials for PD

Reagents and Consumables

| No. | Reagents/Consumables | Vendor | Catalog # |
|---|---|---|---|
| 1 | RIPA Buffer | Sigma | R0278 |
| 2 | Phosphatase Inhibitor Cocktail 2 | Sigma | P5726 |
| 3 | Protease Inhibitor Cocktail | Roche | 04693124001 |
| 4 | Pierce ™ BCA Protein Assay Kit | Thermo | 23225 |
| | | Scientific | |
| 5 | NuPAGE ™ LDS Sample Buffer | Invitrogen | NP0007 |
| | (4×) | | |
| 6 | NuPAGE ™ Sample Reducing Agent | Invitrogen | NP0009 |
| | (10×) | | |
| 7 | NuPAGE ® MES SDS Running | Invitrogen | NP0002 |
| | Buffer (20×) | | |
| 8 | 20 × TBS | Bio-Serve | BS-P-15 |
| 9 | Tween 20 | Sigma | P2287 |
| 10 | PageRuler ™ Prestained Protein | Thermo | 26616 |
| | Ladder | Scientific | |
| 11 | iBlot ® 2 Transfer Stack Regular | Invitrogen | IB23001 |
| 12 | NuPAGE, Novex 4-12% Bis-Tris | Invitrogen | WG1402BOX |
| | Gel, 20well | | |
| 13 | Bovine Serum Albumin | BBI life | A600332-0100 |
| | | sciences | |
| 14 | SuperSignal ™ West Femto | Thermo | 34096 |
| | Maximum Sensitivity Substrate | Scientific | |
| 15 | Nonfat Milk | BD Difco | 232100 |
| 16 | SuperSignal West Pico | Thermo | 34078 |
| | Chemiluminescent Substrate | Scientific | |

1 × TBST: added 50 mL 20 × TBS to 949 mL ddH₂O, and then added 1 mL Tween 20, mixed well.
Running buffer: added 50 mL 20 × MES SDS Running Buffer to 950 mL ddH₂O, mixed well.

Instruments

| No. | Instruments | Vendor | Model # |
|---|---|---|---|
| 1 | Tissuelyser | Shanghai Jingxin | JXFSTPRP-CL |
| 2 | Electro- thermal incubator | Boxun | HPX-9162 MBE |
| 3 | SpectraMax | Molecular | M5e |
| | | Devices | |
| 4 | Dry baths | Hangzhou All | MK-10 |
| | | sheng Instruments | |
| | | Co., Ltd | |
| 5 | Xcell4 SureLock ® | Invitrogen | WR0100 |
| | Midi-cell | | |
| 6 | PowerPac ™ Basic Power | Bio-Rad | 1645050 |
| | Supply | | |
| 7 | Orbital Shaker | Qiling | TS-1 |
| 8 | iBlot ® 2 Gel Transfer | Invitrogen | iBlot 2 |
| | Device | | |
| 9 | Beads | Qiagen | 69989 |
| 10 | Amersham Imager 680 | GE Healthcare | AI680QC |
| 11 | Vortex Shaker | Kylin-Bell | Vortex-6 |
| 12 | Centrifuge | Eppendorf | 5424R |

Antibodies

| No. | Antibodies | Vendor | Catalog # |
|---|---|---|---|
| 1 | BRAF (D9T6S) antibody | Cell Signaling | CST-14814 |
| | | Technology | |
| 2 | phospho-p44/42 MAPK | Cell Signaling | CST-9101 |
| | (Erk½) (Thr202/ | Technology | |
| | Tyr204) antibody | | |
| 3 | GAPDH antibody | Cell signal | CST-5174 |
| | | technology | |
| 4 | Goat Anti-Rabbit | Abcam | ab6721 |
| | IgG H&L (HRP) | | |

### Experimental Procedures

### Cell Culture

The A375 tumor cells were maintained in vitro in DMEM medium supplemented with 100 fetal bovine serum and 1% Penicillin-Streptomycin at 37° C. in an atmosphere of 5% CO₂ in air. The tumor cells were routinely sub-cultured twice weekly. The cells growing in an exponential growth phase were harvested and counted for tumor inoculation. The details of reagents are shown in Table 58.

TABLE 58
Vendor and Cat No. of Cell Line and Reagents

| Cell Line/Reagent | Vendor | Catalog # |
|---|---|---|
| A375 | ATCC | CRL-1619 |
| DMEM | Gibco | 11995-065 |
| Penicillin-Streptomycin | Gibco | 15240-062 |
| 0.25% Trypsin-EDTA | Gibco | 25200-072 |
| FBS | Excell | FSP500 |
| DPBS | Corning | 21-031-CVC |
| Matrigel Matrix | Corning | 354234 |

### Tumor Inoculation

Each mouse was inoculated subcutaneously at the right flank with A375 cells (5×10⁶) in 0.2 mL of PBS supplemented with Matrigel (PBS: Matrigel=1:1) for tumor development. Animals were grouped for the treatment when the average tumor volume reached 341 mm³ for PKPD study on day 18. The test articles administration and the animal numbers in each group are shown in the above experimental Tables 57.

### Reagents Used in the Study

TABLE 59
Vendor and Cat No. of Reagents Used for Formulation Preparation

| Reagent | Vendor | Catalog # |
|---|---|---|
| PEG400 | Sigma | P3265 |
| SBE-β-CD | Kunshan Ruisike | 10059859 |
| | Chemical Materials | |
| HP-β-CD | Energy chemical | E080016 |

### Vehicle Preparation

### Preparation of 25% SBE-β-CD in ddH₂O

SBE-β-CD (50 g) was accurately weighed into a 500 mL glass bottle. Then, 150 mL ddH₂O was added to the bottle and the mixture was stirred to obtain a clear solution. Then ddH₂O was added to a total volume of 200 mL and the mixture was mixed well.

### Preparation of 11.1% HP-β-CD in ddH₂O

HP-β-CD (22.2 g) was accurately weighed into a 500 mL glass bottle. Then, 150 mL ddH₂O was added to the bottle and the mixture was stirred to obtain a clear solution. Then ddH₂O was added to a total volume of 200 mL and the mixture was mixed well.

### Testing Article Formulation Preparation

TABLE 60
Formulation of Dabrafenib Preparation

| | | | | Volume of | Volume of | |
|---|---|---|---|---|---|---|
| | | Conc. of | | PEG400 to be | 11.1% HP-β-CD | |
| Compound | Dose | solution | Compound | added (mL) | to be added | Dosing Solution |
| ID | (mg/kg) | (mg/mL) | amount (mg) | (10%) | (mL) (90%) | observations |
| Dabrafenib^{a} | 100 | 10 | 52.88 | 0.527 | 4.745 | Suspension |

Note:
^{a}The formulation was prepared fresh before using.

TABLE 61
Formulation of Test Compounds Preparation

| | | | | Volume of | Volume of 25% | |
|---|---|---|---|---|---|---|
| | | Conc. of | Compound | PEG400 to be | SBE-β-CD to be | Dosing |
| Compound^{a} | Dose | solution | amount | added (mL) | added (mL) | Solution |
| ID | (mg/kg) | (mg/mL) | (mg) | (20%) | (80%) | observations |
| Compound 26 | 1.5 | 0.15 | 1.48 | 1.946 | 7.784 | Solution |
| Compound 26 | 2 | 0.2 | 2.87 | 2.830 | 11.321 | Solution |
| Compound 26 | 3 | 0.3 | 5.75 | 3.780 | 15.122 | Solution |
| Compound 26 | 6 | 0.6 | 2.99 | 0.983 | 3.932 | Solution |
| Compound 26 | 10 | 1 | 9.93 | 1.959 | 7.834 | Solution |
| Compound 141 | 0.75 | 0.075 | 0.99 | 2.607 | 10.428 | Solution |
| Compound 141 | 1 | 0.1 | 1.57 | 3.101 | 12.403 | Solution |
| Compound 141 | 1.5 | 0.15 | 2.64 | 3.476 | 13.904 | Solution |
| Compound 141 | 3 | 0.3 | 1.49 | 0.981 | 3.924 | Solution |
| Compound 141 | 5 | 0.5 | 4.96 | 1.959 | 7.837 | Solution |

Note:
^{a}The formulation was prepared fresh before using.

### Observations

At the time of routine monitoring, the animals were checked daily for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset. Animals that were observed to be in a continuing deteriorating condition or their tumor size exceeding 3,000 mm³ were euthanized prior to death or before reaching a comatose state.

### Tumor Measurements and the Endpoints

Prior to the onset of drug treatment, mice were measured for tumor size in two dimensions using a caliper, and the tumor volume (mm³) was calculated using formula V=0.5 a×b² where a and b are the long and short diameters of the tumor in mm, respectively. Mice were randomized into different treatment groups based on the tumor volume.

### Experimental Endpoint and Sample Collection

### Plasma Sample Preparation

The plasma samples were collected at predetermined time points for PK analysis. The mice were euthanized at predetermined time points post the treatment. The blood samples were collected into the 1.5 mL Eppendorf tubes containing 3 or 15 μL of 0.5 M EDTA-K2. And then the anticoagulant blood samples were centrifuged at 2,500 g at 4° C. for 15 minutes. The plasma samples were separated into 2 aliquots. The 1^{st} was used for PK analysis, and the 2^{nd} was served as backup.

### Tumor Sample Preparation

The tumor samples were collected at predetermined time points for PK and WB analysis. The tumor was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Brain Sample Preparation

The brain samples were collected at predetermined time points for PK and WB analysis. The brain was dissected out and split into 3 parts (left brain, right brain upper and right brain lower), the left brain was used for PK, the right brain upper was used for WB and the last one was used as the backup for the study. The samples were transferred into pre-labeled tubes and quickly froze in liquid nitrogen within 2 minutes after dissection. The samples were stored at −80° C. before analysis.

### Liver Sample Preparation

Liver samples were collected at predetermined time points for potential PK and WB analysis. The liver was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Lung Sample Preparation

The tumor samples were collected at predetermined time points for potential PK and WB analysis. The lung was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Spleen Sample Preparation

The spleen samples were collected at predetermined time points for potential PK and WB analysis. The spleen was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Skin Sample Preparation

The skin samples were collected at predetermined time points for potential PK and WB analysis. The mice back skin was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Pharmacokinetics Assay

The harvested plasma, tumor and brain samples were processed for pharmacokinetics analysis by LC-MS/MS with a concentration range of 0.500-1000 ng/mL for Dabrafenib in plasma, 0.200-400 ng/mL for Dabrafenib in tumor and brain homogenate, 50.0-50000 ng/mL for Compound 26 in plasma (QD, 1^{st} BID, 2^{nd} BID, 1^{st} TID and 2^{nd} TID (0.25 h-10 h)), 0.500-500 ng/mL for Compound 26 in brain and tumor homogenate, 20.0-20000 ng/mL for Compound 141 in plasma (QD, 1^{st} BID, 2^{nd} BID, 1^{st} TID and 2^{nd} TID (0.25 h-10 h)), 0.200-200 ng/mL for Compound 141 in tumor homogenate, 0.100-100 ng/mL for Compound 141 in brain homogenate, 0.5-500 ng/mL for Compound 141 and Compound 26 in plasma of other.

### Analytical method for Dabrafenib, Compound 26 and Compound 141 in Plasma, Tumor and Brain homogenate

Dabrafenib, Compound 26 and Compound 141 in female BALB/c nude mice plasma (K2-EDTA) tumor and brain homogenate was analyzed by LC-MS/MS (Triple Quad 6500+ Low Mass). Verapamil (5 ng/mL) in acetonitrile: methanol (V: V=95:5) was used as the internal standard for Dabrafenib. Tolbutamide (50 ng/mL) in acetonitrile: methanol (V: V=95:5) was used as the internal standard for Compound 141 and Compound 26 in brain and tumor. Tolbutamide (50 ng/mL) in acetonitrile: methanol (V: V=95:5) was used as the internal standard for Compound 26 in plasma. Tolbutamide (500 ng/mL) in acetonitrile: methanol (V: V=95:5) was used as the internal standard for Compound 141 in plasma (QD, 1^{st} BID, 2^{nd} BID, 1^{st} TID and 2^{nd} TID (0.25 h-10 h)). Tolbutamide (50 ng/mL) in acetonitrile: methanol (V: V=95:5) was used as the internal standard for Compound 141 in plasma of other.

MS condition: ESI positive, MRM detection, Dabrafenib: [M+H]⁺ 519.95/307.00 m/z; Compound 26: [M+H]⁺ 913.31/854.20 m/z; Compound 141: [M+H]⁺ 896.20/837.30 m/z; Tolbutamide (IS): [M+H]⁺ 271.34/91.10 m/z; Verapamil (IS): [M+H]⁺ 455.25/165.20 m/z.

### LC Conditions for Dabrafenib-Plasma, Tumor and Brain Homogenate

Mobile phase A: 0.1% FA in H₂O; mobile phase B: 0.1% FA in ACN;
injection: 2 μL; Column: Waters ACQUITY BEH C8 1.7 μm
2.1*50 mm; Column temperature: 40.0° C.; Flow rate: 0.6 mL/min.

| Time (min) | Mobile Phase B (%) |
|---|---|
| Initial | 30 |
| 0.10 | 30 |
| 0.60 | 85 |
| 1.10 | 100 |
| 1.30 | 100 |
| 1.31 | 30 |
| 1.50 | 30 |

### LC Conditions for Compound 141-Tumor and Brain Homogenate

Mobile phase A: 0.1% FA in H₂O; mobile phase B: 0.1%
FA in ACN; injection: 10 μL for tumor, 15 μL
for brain; Column: Waters ACQUITY BEH C8 1.7 μm
2.1*50 mm; Column temperature: 40.0° C.; Flow rate: 0.6 mL/min.

| Time (min) | Mobile Phase B (%) |
|---|---|
| Initial | 30 |
| 0.10 | 30 |
| 0.60 | 85 |
| 1.10 | 100 |
| 1.30 | 100 |
| 1.31 | 30 |
| 1.50 | 30 |

### LC Conditions for Compound 26-Plasma Homogenate

Mobile phase A: 5 mM HCOONH₄ in H₂O:ACN
(V:V = 95:5); mobile phase B: 5 mM HCOONH₄ in ACN:H₂O
(V:V = 95:5); injection: 2 μL for Compound 26 plasma (QD, 1^{st}
BID, 2^{nd} BID, 1^{st} TID and 2^{nd}
TID (0.25 h-10 h)); 2 μL for Compound 26 plasma of other. Column:
Waters ACQUITY BEH C18 1.7 μm 2.1*50 mm; Column temperature:
40.0° C.; Flow rate: 0.6 mL/min.

| Time (min) | Mobile Phase B (%) |
|---|---|
| Initial | 30 |
| 0.10 | 30 |
| 1.10 | 90 |
| 1.30 | 90 |
| 1.31 | 30 |
| 1.50 | 30 |

### Preparation of Plasma, Brain and Tumor Homogenate Samples

LC conditions for Compound 141-plasma homogenate:
Mobile phase A: 5 mM HCOONH₄ in H₂O:ACN (V:V =
95:5); mobile phase B: 5 mM HCOONH₄ in ACN:H₂O
(V:V = 95:5); injection: 2 μL for Compound 141 plasma (QD,
1^{st} BID, 2^{nd} BID, 1^{st} TID and
2^{nd} TID (0.25 h-10 h)); 10 μL for Compound
141 plasma of other. Column: Waters ACQUITY BEH C18 1.7 μm
2.1*50 mm; Column temperature: 40.0° C.; Flow rate:
0.6 mL/min.

| Time (min) | Mobile Phase B (%) |
|---|---|
| Initial | 20 |
| 0.10 | 20 |
| 1.20 | 95 |
| 1.40 | 95 |
| 1.41 | 20 |
| 1.60 | 20 |

Retention time: Dabrafenib: 0.96 min; Compound 26: 0.69 min; Compound 141: 0.81 min in plasma; Compound 141: 0.79 min in tumor and brain; Verapamil(IS) for Dabrafenib: 0.80 min; Tolbutamide (IS) for Compound 141 in plasma: 0.82 min; Tolbutamide (IS) for Compound 141 in tumor and brain: 0.89 min; Tolbutamide (IS) for Compound 26: 0.70 min.

An aliquot of 20 μL unknown sample, calibration standard, quality control, dilute quality control, single blank and double blank samples were added to the 96-well plate. Each sample for Dabrafenib in plasma(except the double blank) was quenched with 400 μL IS respectively (double blank sample was quenched with 400 μL ACN:MeOH (V: V=95:5)), each sample for Compound 141 in plasma (QD, 1^{st} BID, 2^{nd} BID, 1^{st} TID and 2^{nd} TID (0.25 h-10 h)) and Compound 26 in plasma (QD, 1^{st} BID, 2^{nd} BID, 1^{st} TID and 2^{nd} TID (0.25 h-10 h)) (except the double blank) was quenched with 400 μL IS respectively (double blank sample was quenched with 400 μL ACN: MeOH (V: V=95:5)), each sample for Dabrafenib and Compound 26 in tumor and brain(except the double blank) was quenched with 200 μL IS respectively (double blank sample was quenched with 200 μL ACN:MeOH (V: V=95:5)), each sample for Compound 141 and Compound 26 in plasma of other (except the double blank) was quenched with 200 μL IS respectively (double blank sample was quenched with 200 μL ACN: MeOH (V: V=95:5)), each sample for Compound 141 in tumor and brain(except the double blank) was quenched with 100 μL IS respectively (double blank sample was quenched with 100 μL ACN: MeOH (V: V=95:5)), and then the mixture was vortex-mixed for 5 min at 800 rpm and centrifuged for 15 min at 3220 g (4000 rpm), 4° C. 30 μL supernatant was diluted with 30 μL of H₂O for Compound 141 and Dabrafenib in plasma, brain and tumor, 30 μL supernatant was diluted with 30 μL of H₂O for Compound 26 in brain and tumor, 30 μL supernatant was diluted with 30 μL of H₂O for Compound 26 in plasma of other, 20 μL supernatant was diluted with 100 μL of H₂O:ACN (V:V=75:25) for Compound 26 in plasma (QD, 1^{st} BID, 2^{nd} BID, 1^{st} TID and 2^{nd} TID (0.25 h-10 h)), the diluted solution were directly injected for LC-MS/MS analysis.

Dilution procedure: When dilution factor is 10, an aliquot of 2 μL sample was added with 18 μL blank matrix; when dilution factor is 100, an aliquot of 2 μL sample was added with 18 μL blank matrix; and then an aliquot of 2 μL sample was added with 18 μL blank matrix.

Calibration curve: 20.0-20000 ng/mL for Compound 141 in plasma (QD, 1^{st} BID, 2^{nd} BID, 1^{st} TID and 2^{nd} TID (0.25 h-10 h)); 0.5-500 ng/mL for Compound 141 and Compound 26 in plasma of other, 50.0-50000 ng/mL for Compound 26 in plasma (QD, 1^{st} BID, 2^{nd} BID, 1^{st} TID and 2^{nd} TID (0.25 h-10 h)), 0.500-1000 ng/mL for Dabrafenib in plasma, 0.200-200 ng/mL for Compound 141 in tumor, 0.100-100 ng/mL for Compound 141 in brain, 0.500-500 ng/mL for Compound 26 in brain and tumor, 0.200-400 ng/mL for Dabrafenib in tumor and brain.

### Western Blotting Assay

### Protein Extraction & Protein Quantification

The previously snap-frozen tissues were removed from the −80° C. freezer and then cut up to about 30-100 mg of tissue per sample. The tissues were then placed in 2 mL microcentrifuge tube and 400 μL of RIPA Buffer was then added. The RIPA Buffer contained 1% Protease Inhibitor Cocktail and 1% Phosphatase Inhibitor Cocktail 2.

The tumors were then ground with a freeze grinder at 50 Hz for five minutes and then the tissue lysates were kept on ice for thirty minutes. The lysates were shaken every five minutes. The lysates were then centrifuged at 15,000 rpm at 4° C. for ten minutes and the supernatants were gently transferred to a new microcentrifuge tube kept on ice.

The protein concentrations were measured with Pierce™ BCA Protein Assay Kit. Then the samples were diluted, according to the result of BCA protein quantification, to the same final concentration (at 4 μg/μL) using RIPA buffer plus 4× LDS Sample Buffer and 10× Sample Reducing Agent. The samples were then heated at 100° C. for ten minutes. The denatured samples were then used for Western blotting or kept in the −80° C. refrigerator for long term storage.

### Western Blotting

The protein samples were loaded to NuPAGE® Novex 4-12% Bis-Tris gel with 10 μL per slot. The electrophoresis was ran with MES running buffer at 80 V for 30 minutes and then at 120 V for 90 minutes. Then protein was transferred to NC (nitrocellulose) membranes with iBlot®2 Gel Transfer Device, P3 for seven minutes. Afterwards, the membranes were cut, the interest membranes parts were kept and washed with 5-10 mL 1× TBST three times for 5 minutes each. Then the non-specific protein was blocked by 5-10 mL 1× TBST with 5% nonfat milk at room temperature for one hour. The membrane was then washed with 5-10 mL 1× TBST three times for five minutes each. The membrane was then incubated with 5-10 mL diluted primary antibody (BRAF antibody was diluted at a ratio of 1:1000, phospho-p44/42 MAPK antibody was diluted at a ratio of 1:2000 and GAPDH antibody was diluted at a ratio of 1:2000) in 1× TBST with 5% BSA at 4° C. with gentle agitation overnight. Next, the membrane was washed with 5-10 mL 1× TBST three times for ten minutes each. The resulting membrane was incubated with 5-10 mL HRP-conjugated secondary antibody (generally 1:3000) with gentle agitation for one hour at room temperature. Then the membrane was washed with 5-10 mL 1× TBST three times for ten minutes each. Afterwards, the HRP substrate from West Femto Maximum Sensitivity kit was added to the NC membranes and chemiluminescence was detected by AI680QC.

### Quantification of Band Intensity

Intensity of individual bands were quantified using Image Studio Lite Ver 5.2 software.

### Statistical Analysis

Band intensity of individual bands was obtained for analysis. Protein expression was quantitated in relation to a reference protein, and then the normalized folds of protein expression in each sample were ratios to vehicle group.

The normalized data were interpreted by GraphPad Prism. Statistical analysis of difference in the protein expression of interest among the groups was conducted.

### Results

### Results for PK and PD Study

### Pharmacokinetics Results

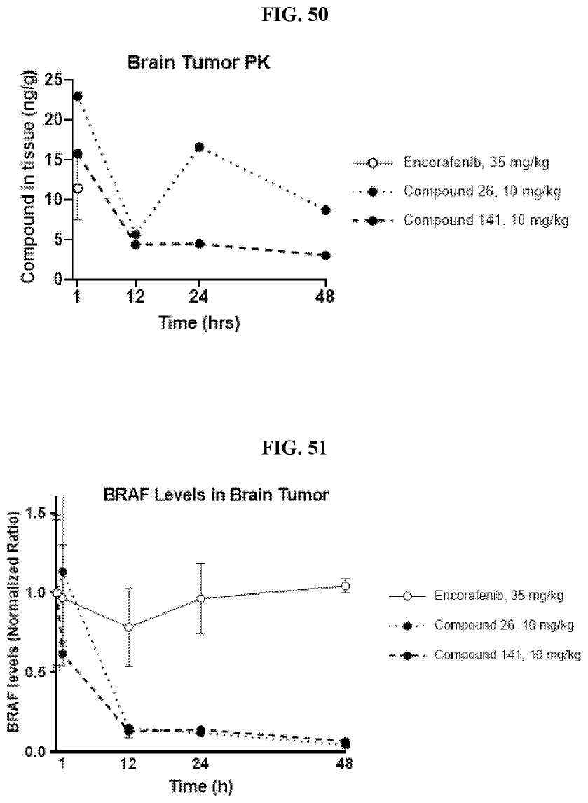

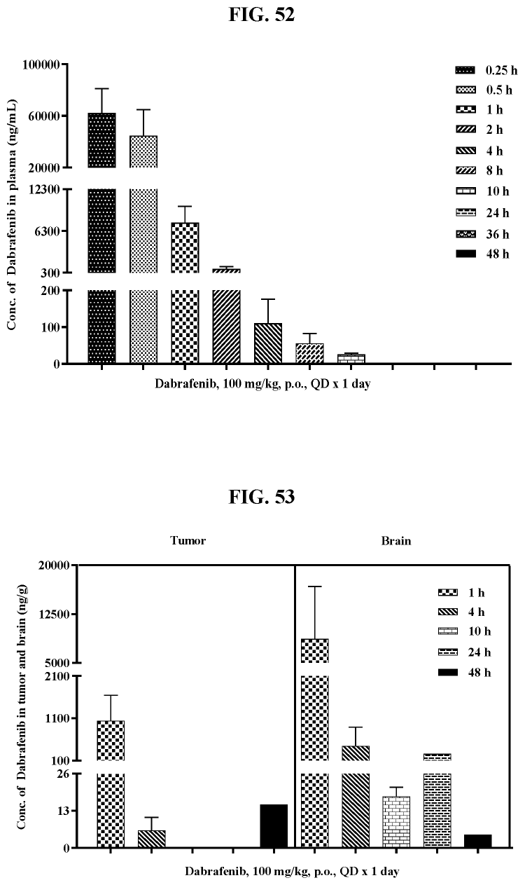

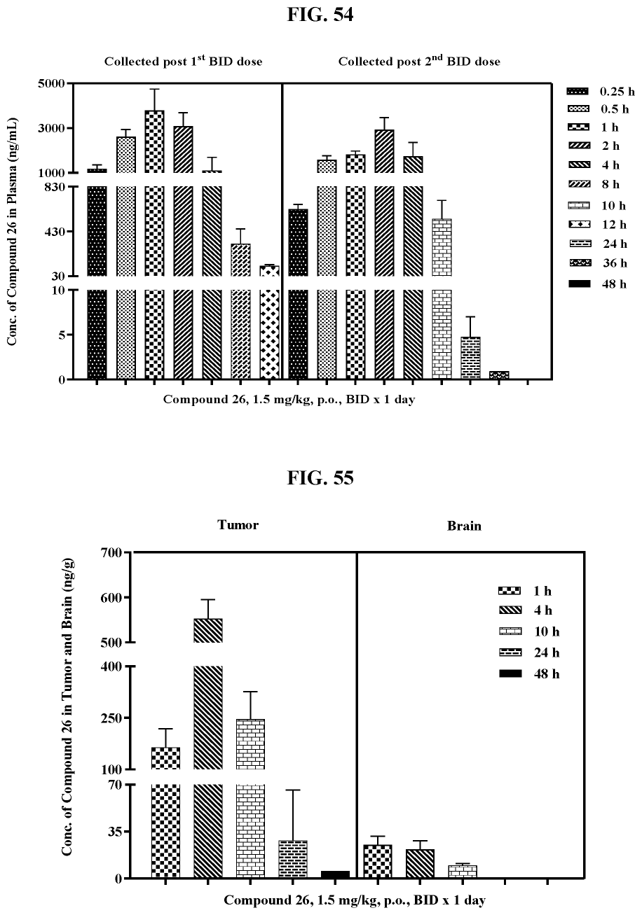

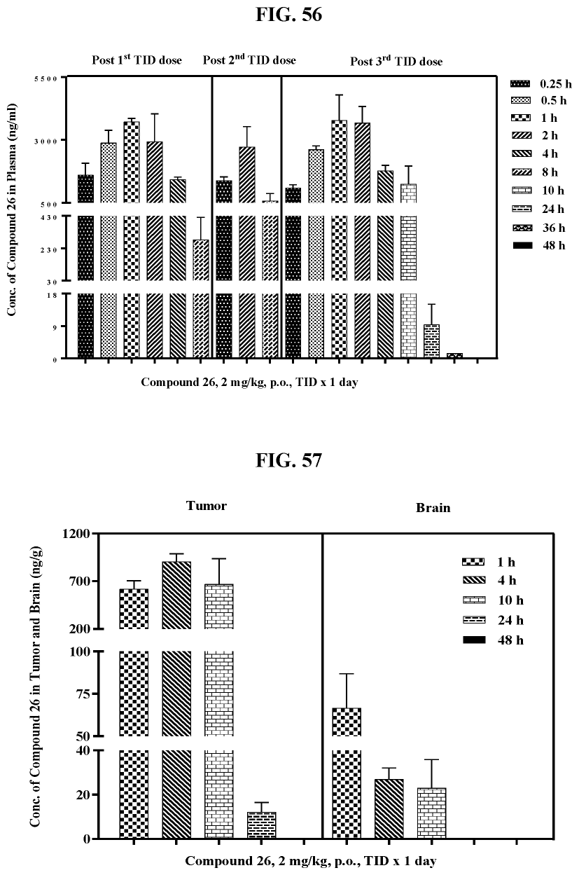

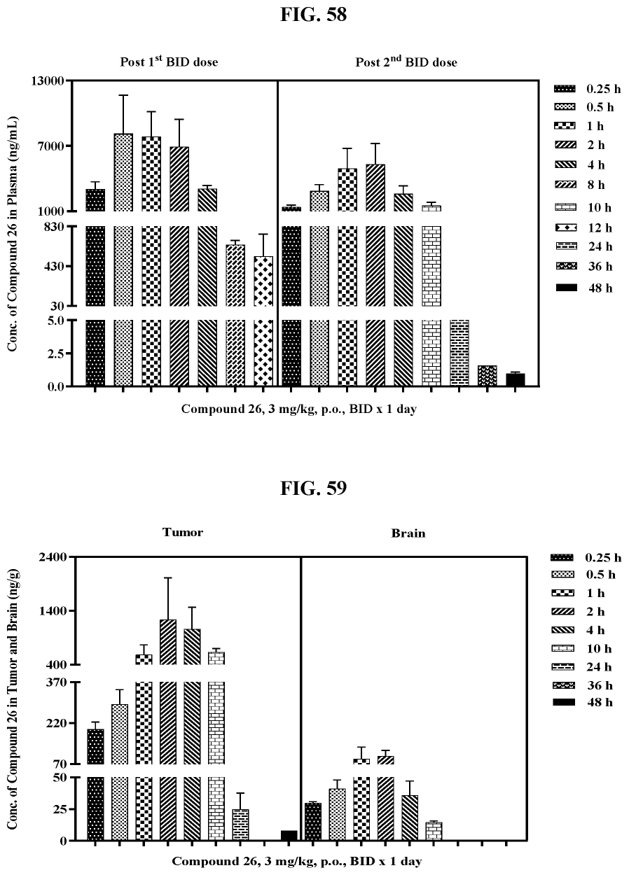

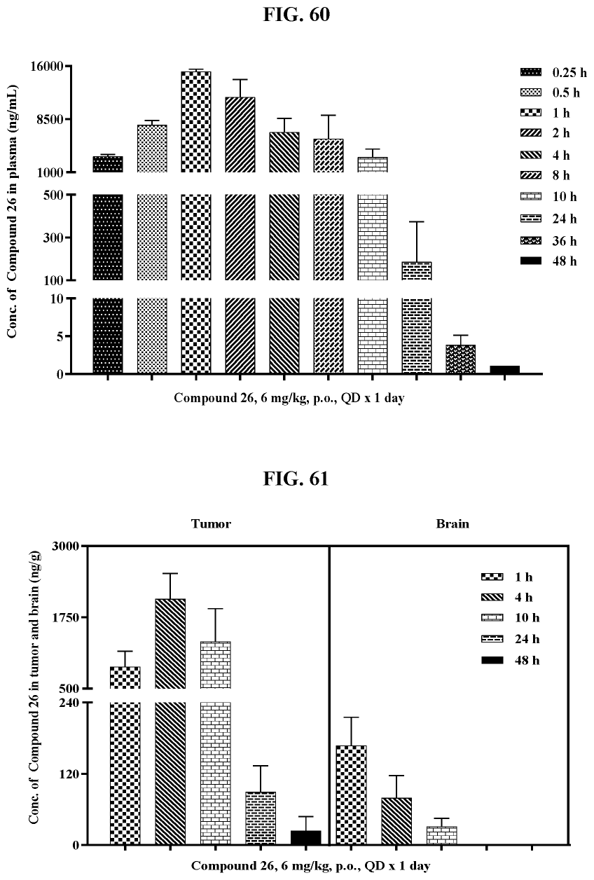

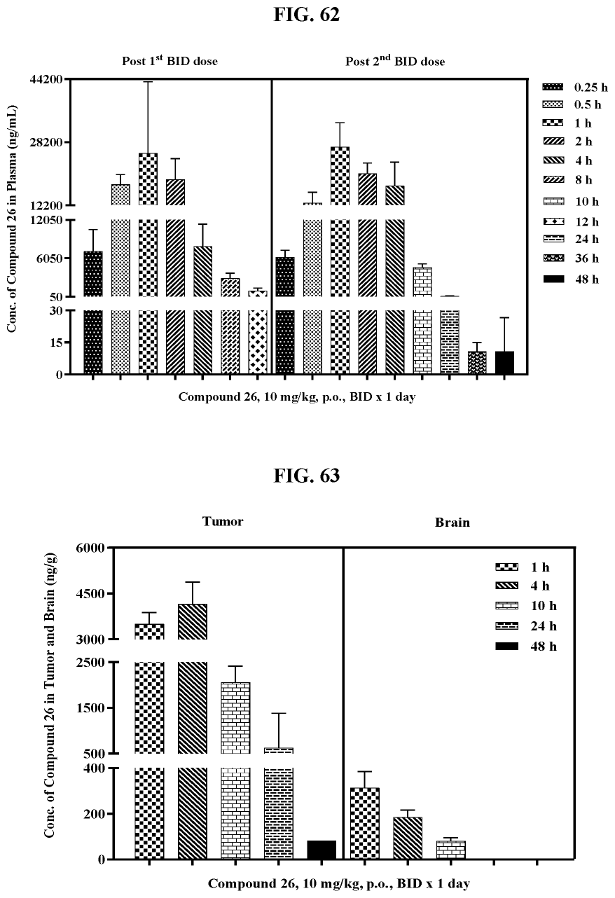

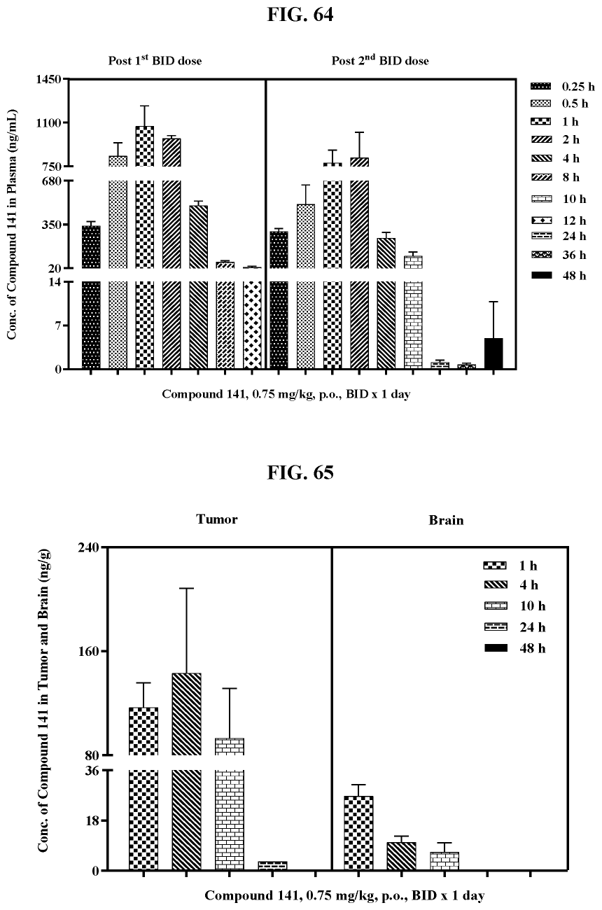

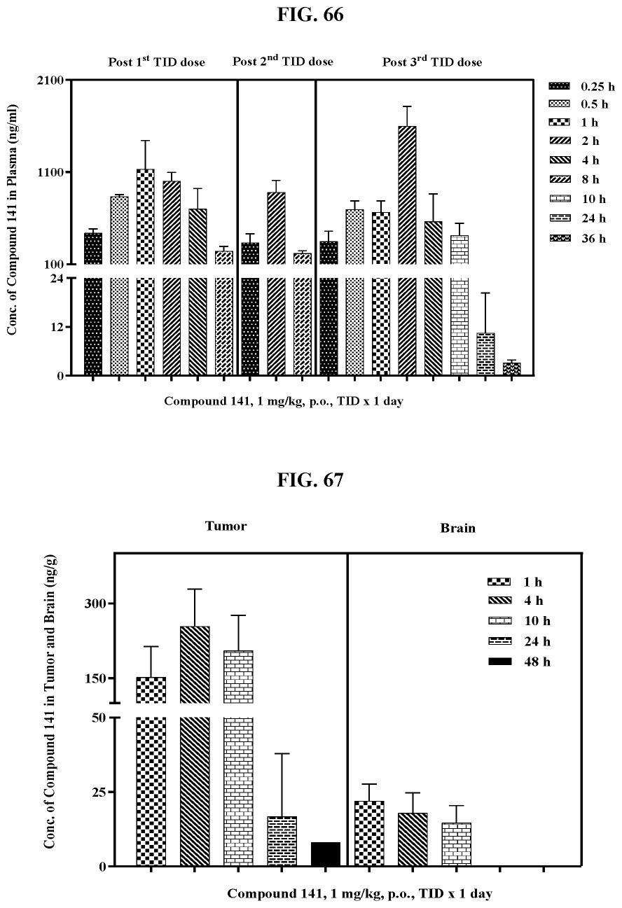

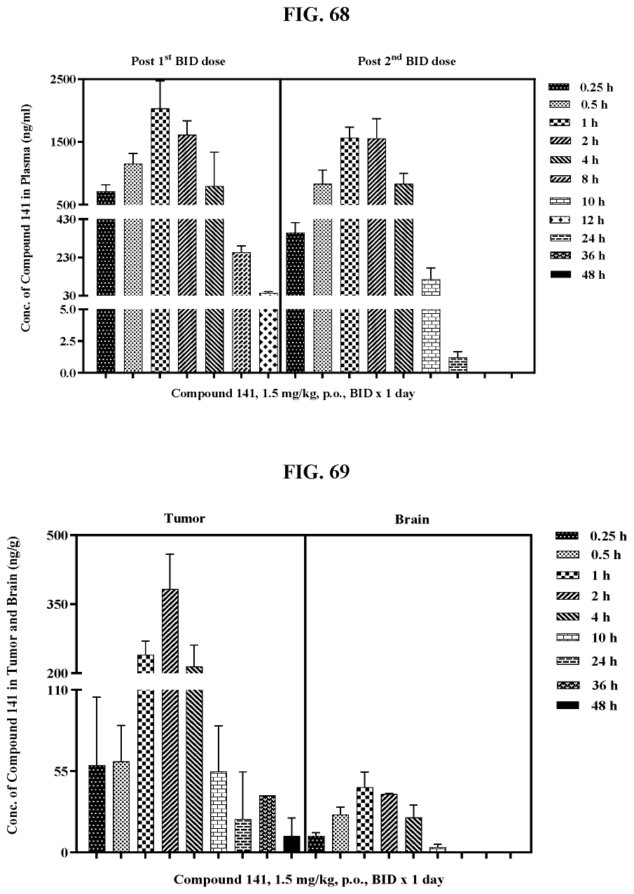

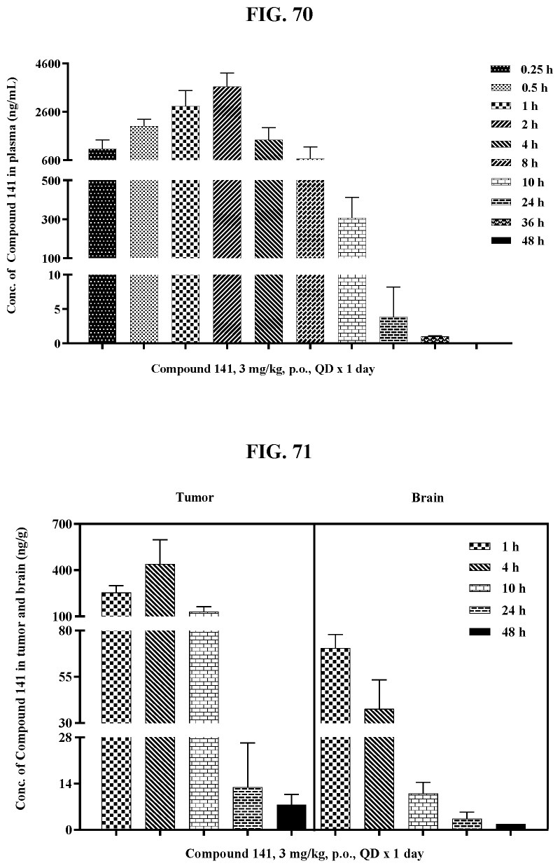

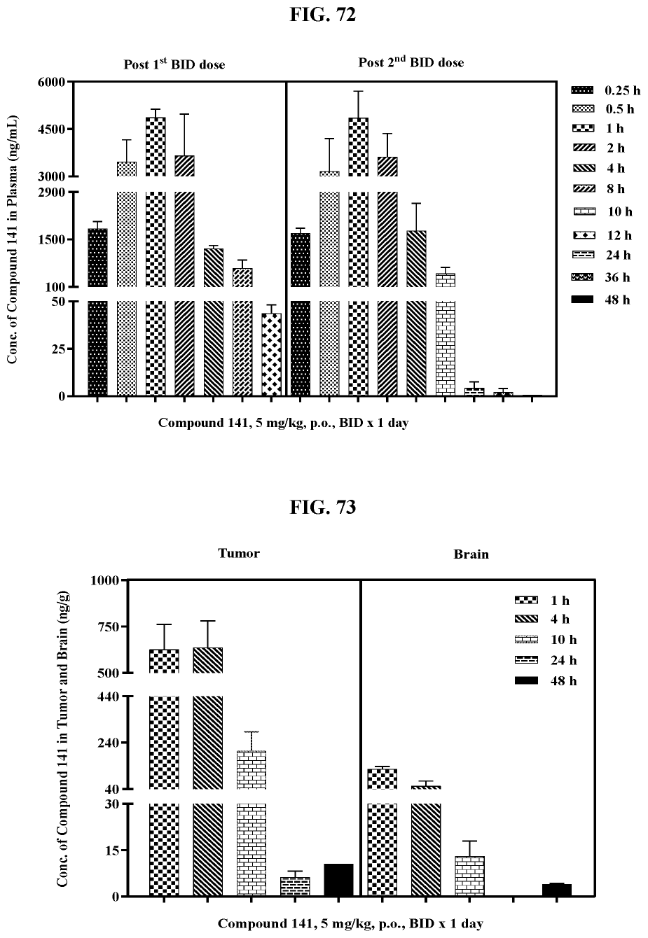

The concentrations of Dabrafenib, Compound 26 and Compound 141 in the treatment of female BALB/c nude mice bearing A375 xenografts are shown in Table 62-Table 72 and FIG. 52-FIG. 73. The AUC results are shown in Table 73 and Table 74.

TABLE 62
Mean Concentration of Dabrafenib in Plasma, Tumor and Brain

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 14, | 0.25 | h | 62233 ± 10894 | / | / |
| Dabrafenib, | 0.5 | h | 44700 ± 11600 | / | / |
| 100 mg/kg, p.o., | 1 | h | 7487 ± 1358 | 8657 ± 4660 | 1040 ± 349 |
| QD × 1 day | 2 | h | 844 ± 198 | / | / |
| | 4 | h | 110 ± 37.6 | 442 ± 257 | 6.13 ± 0 |
| | 8 | h | 55.6 ± 15.5 | / | / |
| | 10 | h | 25.9 ± 2.09 | 18.0 ± 1.88 | BQL^{a} |
| | 24 | h | 0.616 ± 0 | 253 ± 0 | BQL |
| | 36 | h | BQL | / | / |
| | 48 | h | BQL | 4.66 ± 0 | 15.2 ± 0 |

Note:
^{a}BQL: Below the lower limit of quantitation.

TABLE 63
Mean Concentration of Compound 26 in Plasma, Tumor
and Brain of Compound 26 (1.5 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 15 | 0.25 | h | 1183 ± 103 | / | / |
| Compound 26, 1.5 mg/kg, | 0.5 | h | 2617 ± 182 | / | / |
| p.o., BID × 1 day | 1 | h | 3787 ± 553 | / | / |
| Collected post 1^{st} | 2 | h | 3087 ± 346 | / | / |
| BID dose | 4 | h | 1108 ± 339 | / | / |
| | 8 | h | 318 ± 78.2 | / | / |
| | 12 | h | 121 ± 6.74 | / | / |
| Group 15 | 0.25 | h | 629 ± 24.4 | / | / |
| Compound 26, 1.5 mg/kg, | 0.5 | h | 1577 ± 108 | / | / |
| p.o., BID × 1 day | 1 | h | 1820 ± 86.6 | 164 ± 31.1 | 25.0 ± 3.74 |
| Collected post 2^{nd} | 2 | h | 2930 ± 312 | / | / |
| BID dose | 4 | h | 1737 ± 355 | 552 ± 24.7 | 21.8 ± 3.64 |
| | 10 | h | 541 ± 95.8 | 246 ± 46.4 | 9.78 ± 0.841 |
| | 24 | h | 4.74 ± 1.31 | 28.3 ± 21.8 | BQL^{a} |
| | 36 | h | 0.889 ± 0 | / | / |
| | 48 | h | BQL | 5.63 ± 0 | BQL |

Note:
^{a}BQL: Below the lower limit of quantitation.

TABLE 64
Mean Concentration of Compound 26 in Plasma, Tumor
and Brain of Compound 26 (2 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 16 | 0.25 | h | 1600 ± 272 | / | / |
| Compound 26, 2 mg/kg, | 0.5 | h | 2880 ± 289 | / | / |
| p.o., TID × 1 day | 1 | h | 3720 ± 77.7 | / | / |
| Collected post 1^{st} | 2 | h | 2930 ± 635 | / | / |
| TID dose | 4 | h | 1423 ± 56.7 | / | / |
| | 8 | h | 283 ± 80.3 | / | / |
| Group 16 | 0.25 | h | 1387 ± 84.1 | | |
| Compound 26, 2 mg/kg, | 2 | h | 2720 ± 466 | | |
| p.o., TID × 1 day | 8 | h | 580 ± 166 | | |
| Collected post 2^{nd} | | | | | |
| TID dose | | | | | |
| Group 16 | 0.25 | h | 1086 ± 75.2 | / | / |
| Compound 26, 2 mg/kg, | 0.5 | h | 2627 ± 81.7 | / | / |
| p.o., TID × 1 day | 1 | h | 3780 ± 584 | 617 ± 49.7 | 66.5 ± 11.7 |
| Collected post 3^{rd} | 2 | h | 3677 ± 376 | / | / |
| TID dose | 4 | h | 1773 ± 127 | 905 ± 48.2 | 26.9 ± 2.99 |
| | 10 | h | 1250 ± 411 | 667 ± 154 | 23.0 ± 7.44 |
| | 24 | h | 9.4 ± 3.31 | 12.0 ± 2.59 | BQL^{a} |
| | 36 | h | 1.45 ± 0 | / | / |
| | 48 | h | BQL | BQL | BQL |

Note:
^{a}BQL: Below the lower limit of quantitation.

TABLE 65
Mean Concentration of Compound 26 in Plasma, Tumor
and Brain of Compound 26 (3 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 17 | 0.25 | h | 3010 ± 401 | / | / |
| Compound 26, 3 mg/kg, | 0.5 | h | 8137 ± 2034 | / | / |
| p.o., BID × 1 day | 1 | h | 7840 ± 1332 | / | / |
| Collected post 1^{st} | 2 | h | 6907 ± 1452 | / | / |
| BID dose | 4 | h | 3063 ± 172 | / | / |
| | 8 | h | 645 ± 24.7 | / | / |
| | 12 | h | 527 ± 130 | / | / |
| Group 17 | 0.25 | h | 1380 ± 113 | 197 ± 15.3 | 29.7 ± 0 |
| Compound 26, 3 mg/kg, | 0.5 | h | 2883 ± 325 | 289 ± 31.1 | 41.0 ± 4.05 |
| p.o., BID × 1 day | 1 | h | 4920 ± 1062 | 581 ± 107 | 89.1 ± 25.0 |
| Collected post 2^{nd} | 2 | h | 5293 ± 1109 | 1232 ± 452 | 99.5 ± 11.9 |
| BID dose | 4 | h | 2593 ± 422 | 1059 ± 233 | 35.8 ± 6.52 |
| | 10 | h | 1503 ± 194 | 630 ± 39.9 | 14.5 ± 0.737 |
| | 24 | h | 20.5 ± 4.61 | 24.7 ± 7.50 | BQL^{a} |
| | 36 | h | 1.58 ± 0 | BQL | BQL |
| | 48 | h | 0.968 ± 0.0751 | 7.94 ± 0 | BQL |

Note:
^{a}BQL: Below the lower limit of quantitation.

TABLE 66
Mean Concentration of Compound 26 in Plasma, Tumor
and Brain of Compound 26 (6 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 18, | 0.25 | h | 3250 ± 159 | / | / |
| Compound 26, | 0.5 | h | 7683 ± 353 | / | / |
| 6 mg/kg, p.o., | 1 | h | 15233 ± 186 | 882 ± 157 | 167 ± 27.6 |
| QD × 1 day | 2 | h | 11600 ± 1450 | / | / |
| | 4 | h | 6670 ± 1114 | 2070 ± 259 | 79.2 ± 21.8 |
| | 8 | h | 5720 ± 1944 | / | / |
| | 10 | h | 3113 ± 682 | 1324 ± 333 | 30.8 ± 8.11 |
| | 24 | h | 185 ± 109 | 89.1 ± 25.7 | BQL^{a} |
| | 36 | h | 3.87 ± 0.735 | / | / |
| | 48 | h | 1.07 ± 0 | 24.2 ± 0 | BQL |

Note:
^{a}BQL: Below the lower limit of quantitation.

TABLE 67
Mean Concentration of Compound 26 in Plasma, Tumor
and Brain of Compound 26 (10 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 19 | 0.25 | h | 7140 ± 1957 | / | / |
| Compound 26, 10 mg/kg, | 0.5 | h | 17433 ± 1466 | / | / |
| p.o., BID × 1 day | 1 | h | 25400 ± 10453 | / | / |
| Collected post 1^{st} | 2 | h | 18767 ± 3021 | / | / |
| BID dose | 4 | h | 7887 ± 2012 | / | / |
| | 8 | h | 2917 ± 468 | / | / |
| | 12 | h | 964 ± 248 | / | / |
| Group 19 | 0.25 | h | 6223 ± 623 | / | / |
| Compound 26, 10 mg/kg, | 0.5 | h | 12760 ± 1588 | / | / |
| p.o., BID × 1 day | 1 | h | 27000 ± 3528 | 3497 ± 219 | 313 ± 41.7 |
| Collected post 2^{nd} | 2 | h | 20233 ± 1545 | / | / |
| BID dose | 4 | h | 17133 ± 3467 | 4163 ± 413 | 185 ± 18.2 |
| | 10 | h | 4637 ± 318 | 2057 ± 207 | 80.4 ± 8.59 |
| | 24 | h | 143 ± 29.1 | 624 ± 438 | BQL^{a} |
| | 36 | h | 10.7 ± 2.46 | / | / |
| | 48 | h | 10.64 ± 9.23 | 81.6 ± 0 | BQL |

Note:
^{a}BQL: Below the lower limit of quantitation.

TABLE 68
Mean Concentration of Compound 141 in Plasma, Tumor
and Brain of Compound 141 (0.75 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 20 | 0.25 | h | 340 ± 19.0 | / | / |
| Compound 141, 0.75 mg/kg, | 0.5 | h | 832 ± 61.1 | / | / |
| p.o., BID × 1 day | 1 | h | 1071 ± 94.7 | / | / |
| Collected post 1st | 2 | h | 972 ± 13.5 | / | / |
| BID dose | 4 | h | 491 ± 20.2 | / | / |
| | 8 | h | 66.3 ± 5.93 | / | / |
| | 12 | h | 26.5 ± 4.92 | / | / |
| Group 20 | 0.25 | h | 297 ± 13.2 | / | / |
| Compound 141, 0.75 mg/kg, | 0.5 | h | 502 ± 84.1 | / | / |
| p.o., BID × 1 day | 1 | h | 778 ± 59.4 | 117 ± 11.0 | 26.7 ± 2.39 |
| Collected post 2nd | 2 | h | 818 ± 118 | / | / |
| BID dose | 4 | h | 247 ± 24.5 | 143 ± 37.7 | 10.2 ± 1.30 |
| | 10 | h | 111 ± 18.4 | 93.0 ± 22.1 | 6.61 ± 2.00 |
| | 24 | h | 1.09 ± 0.203 | 3.27 ± 0 | BQL^{a} |
| | 36 | h | 0.764 ± 0 | / | / |
| | 48 | h | 4.96 ± 0 | BQL | BQL |

Note:
^{a}BQL: Below the lower limit of quantitation.

TABLE 69
Mean Concentration of Compound 141 in Plasma,
Tumor and Brain of Compound 141 (1 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 21 | 0.25 | h | 438 ± 25.4 | / | / |
| Compound 141, 1 mg/kg, | 0.5 | h | 836 ± 10.8 | / | / |
| p.o., TID × 1 day | 1 | h | 1130 ± 180 | / | / |
| Collected post 1^{st} | 2 | h | 1002 ± 55.2 | / | / |
| TID dose | 4 | h | 699 ± 129 | / | / |
| | 8 | h | 242 ± 30.4 | / | / |
| Group 21 | 0.25 | h | 334 ± 54.7 | | |
| Compound 141, 1 mg/kg, | 2 | h | 879 ± 74.6 | | |
| p.o., TID × 1 day | 8 | h | 218 ± 16.3 | | |
| Collected post 2^{nd} | | | | | |
| TID dose | | | | | |
| Group 21 | 0.25 | h | 346 ± 65.5 | / | / |
| Compound 141, 1 mg/kg, | 0.5 | h | 694 ± 54.4 | / | / |
| p.o., TID × 1 day | 1 | h | 664 ± 72 | 152 ± 35.7 | 22.0 ± 3.26 |
| Collected post 3^{rd} | 2 | h | 1597 ± 124 | / | / |
| TID dose | 4 | h | 563 ± 173 | 254 ± 43.2 | 17.9 ± 3.94 |
| | 10 | h | 415 ± 74.4 | 205 ± 40.9 | 14.6 ± 3.36 |
| | 24 | h | 10.5 ± 5.67 | 16.7 ± 12.2 | BQL^{a} |
| | 36 | h | 3.20 ± 0.392 | / | / |
| | 48 | h | BQL | 8.07 ± 0 | BQL |

Note:
^{a}BQL: Below the lower limit of quantitation.

TABLE 70
Mean Concentration of Compound 141 in Plasma, Tumor
and Brain of Compound 141 (1.5 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 22 | 0.25 | h | 710 ± 61.9 | / | / |
| Compound 141, 1.5 mg/kg, | 0.5 | h | 1152 ± 94.0 | / | / |
| p.o., BID × 1 day | 1 | h | 2030 ± 255 | / | / |
| Collected post 1^{st} | 2 | h | 1613 ± 128 | / | / |
| BID dose | 4 | h | 796 ± 312 | / | / |
| | 8 | h | 256 ± 19.3 | / | / |
| | 12 | h | 43.4 ± 4.65 | / | / |
| Group 22 | 0.25 | h | 359 ± 30.7 | 58.9 ± 26.7 | 11.2 ± 1.24 |
| Compound 141, 1.5 mg/kg, | 0.5 | h | 830 ± 129 | 61.5 ± 14.0 | 25.5 ± 2.96 |
| p.o., BID × 1 day | 1 | h | 1563 ± 99.1 | 239 ± 17.6 | 44.1 ± 5.96 |
| Collected post 2^{nd} | 2 | h | 1553 ± 181 | 382 ± 43.9 | 39.3 ± 0.318 |
| BID dose | 4 | h | 831 ± 96.6 | 214 ± 27.1 | 23.5 ± 4.95 |
| | 10 | h | 115 ± 34.3 | 54.7 ± 17.9 | 3.51 ± 1.17 |
| | 24 | h | 1.20 ± 0.262 | 22.5 ± 18.5 | BQL^{a} |
| | 36 | h | BQL | 38.5 ± 0 | BQL |
| | 48 | h | BQL | 11.1 ± 0 | BQL |

Note:
^{a}BQL: Below the lower limit of quantitation.

TABLE 71
Mean Concentration of Compound 141 in Plasma,
Tumor and Brain of Compound 141 (3 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 23, | 0.25 | h | 1068 ± 209 | / | / |
| Compound 141, 3 mg/kg, | 0.5 | h | 2017 ± 160 | / | / |
| p.o., QD × 1 day | 1 | h | 2833 ± 372 | 254 ± 26.3 | 70.5 ± 4.29 |
| | 2 | h | 3657 ± 315 | / | / |
| | 4 | h | 1444 ± 285 | 439 ± 90.9 | 37.6 ± 9.11 |
| | 8 | h | 661 ± 279 | / | / |
| | 10 | h | 307 ± 60.4 | 130 ± 19.0 | 11.0 ± 1.97 |
| | 24 | h | 3.86 ± 2.50 | 12.9 ± 7.75 | 3.31 ± 0 |
| | 36 | h | 1.05 ± 0 | / | / |
| | 48 | h | BQL^{a} | 7.64 ± 0 | 1.79 ± 0 |

Note:
^{a}BQL: Below the lower limit of quantitation.

TABLE 72
Mean Concentration of Compound 141 in Plasma,
Tumor and Brain of Compound 141 (5 mg/kg) Group

| | | | Concentration | Concentration | Concentration |
|---|---|---|---|---|---|
| | Sample | | in Plasma | in Tumor | in Brain |
| Group | time | | (ng/mL) ± SEM | (ng/g) ± SEM | (ng/g) ± SEM |
| Group 24 | 0.25 | h | 1813 ± 126 | / | / |
| Compound 141, 5 mg/kg, | 0.5 | h | 3460 ± 404 | / | / |
| p.o., BID × 1 day | 1 | h | 4860 ± 155 | / | / |
| Collected post 1st | 2 | h | 3653 ± 760 | / | / |
| BID dose | 4 | h | 1223 ± 55.5 | / | / |
| | 8 | h | 648 ± 142.3 | / | / |
| | 12 | h | 43.6 ± 2.70 | / | / |
| Group 24 | 0.25 | h | 1680 ± 89.6 | / | / |
| Compound 141, 5 mg/kg, | 0.5 | h | 3160 ± 596 | / | / |
| p.o., BID × 1 day | 1 | h | 4853 ± 488 | 626 ± 78.6 | 126 ± 6.12 |
| Collected post 2nd | 2 | h | 3620 ± 428 | / | / |
| BID dose | 4 | h | 1753 ± 468 | 636 ± 83.4 | 52.9 ± 12.8 |
| | 10 | h | 484 ± 109 | 205 ± 47.2 | 13.1 ± 2.79 |
| | 24 | h | 4.28 ± 1.89 | 6.26 ± 1.12 | BQL^{a} |
| | 36 | h | 2.06 ± 1.17 | / | / |
| | 48 | h | 0.635 ± 0 | 10.6 ± 0 | 4.00 ± 0 |

Note:
^{a}BQL: Below the lower limit of quantitation.

TABLE 73
Plasma AUC

AUC at 24 hours (h* ng/mL)

| | Average | SD |
|---|---|---|
| Dabrafenib, 100 | 32131 | 6615 |
| mg/kg, p.o., QD | | |
| Compound 26, 6 | 85121 | 19373 |
| mg/kg, p.o., QD | | |
| Compound 26, 1.5 | 27630 | 1007 |
| mg/kg, p.o., BID | | |
| Compound 26, 3 | 60168 | 2513 |
| mg/kg, p.o., BID | | |
| Compound 26, 10 | 223984 | 14961 |
| mg/kg, p.o., BID | | |
| Compound 26, 2 | 41846 | 4544 |
| mg/kg, p.o., TID | | |

TABLE 74
Brain and Tumor AUC

AUC at 24 hours
(h*ng/mL)

| | | Average | SD |
|---|---|---|---|
| Group 14: Dabrafenib, 100 | Brain | 1578 | 888 |
| mg/kg, p.o., QD × 1 day | | | |
| Group 14: Dabrafenib, 100 | Tumor | 1678 | 966 |
| mg/kg, p.o., QD × 1 day | | | |
| Group 15: Compound 26, 1.5 | Brain | 233 | 40 |
| mg/kg, p.o., BID × 1 day | | | |
| Group 16: Compound 26, 2 | Brain | 451 | 154 |
| mg/kg, p.o., TID × 1 day | | | |
| Group 17: Compound 26, 3 | Brain | 522 | 34 |
| mg/kg, p.o., BID × 1 day | | | |
| Group 18: Compound 26, 6 | Brain | 915 | 243 |
| mg/kg, p.o., QD × 1 day | | | |
| Group 19: Compound 26, 10 | Brain | 2104 | 161 |
| mg/kg, p.o., BID × 1 day | | | |
| Group 15: Compound 26, 1.5 | Tumor | 5752 | 1421 |
| mg/kg, p.o., BID × 1 day | | | |
| Group 16: Compound 26, 2 | Tumor | 11899 | 3190 |
| mg/kg, p.o., TID × 1 day | | | |
| Group 17: Compound 26, 3 | Tumor | 13291 | 1816 |
| mg/kg, p.o., BID × 1 day | | | |
| Group 18: Compound 26, 6 | Tumor | 25765 | 5107 |
| mg/kg, p.o., QD × 1 day | | | |
| Group 19: Compound 26, 10 | Tumor | 56735 | 17878 |
| mg/kg, p.o., BID × 1 day | | | |

### Western Blotting Analysis Results

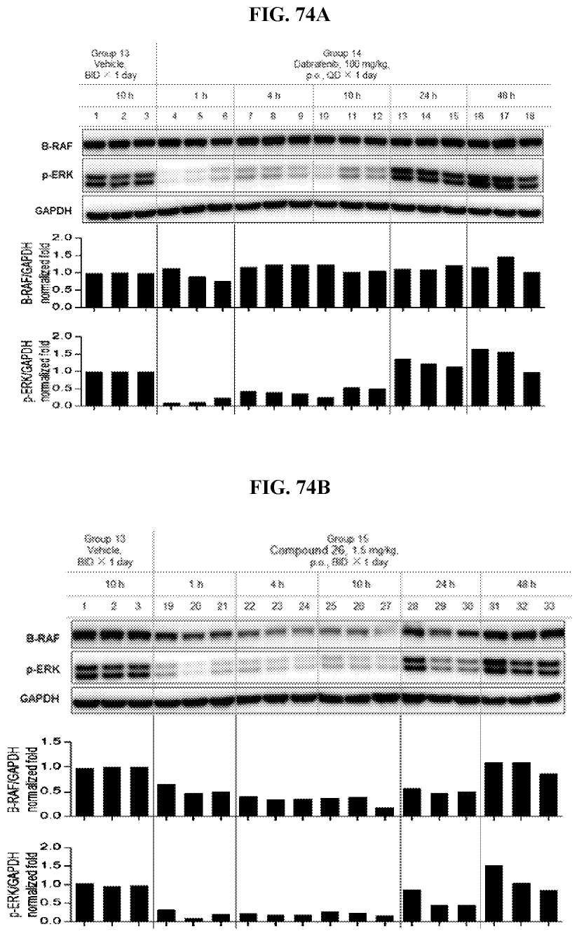

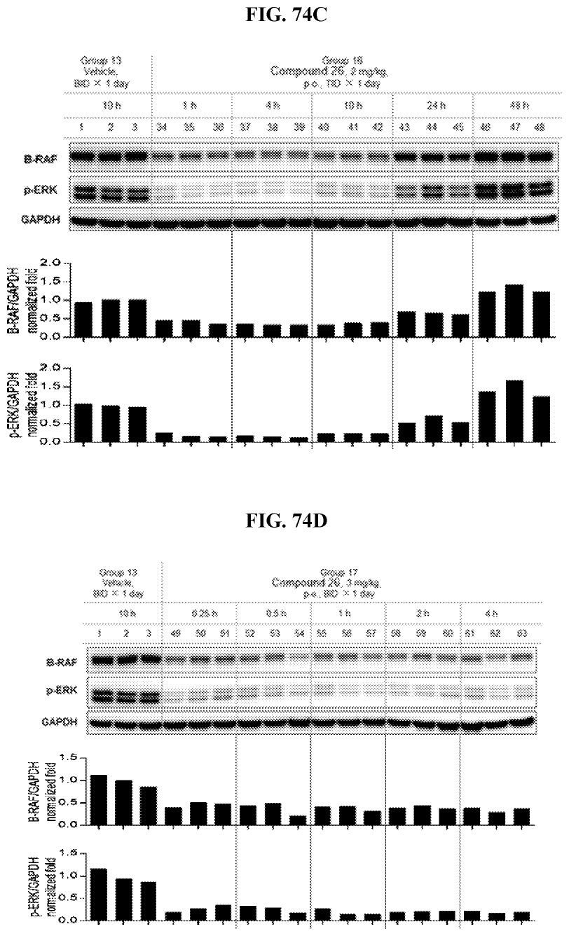

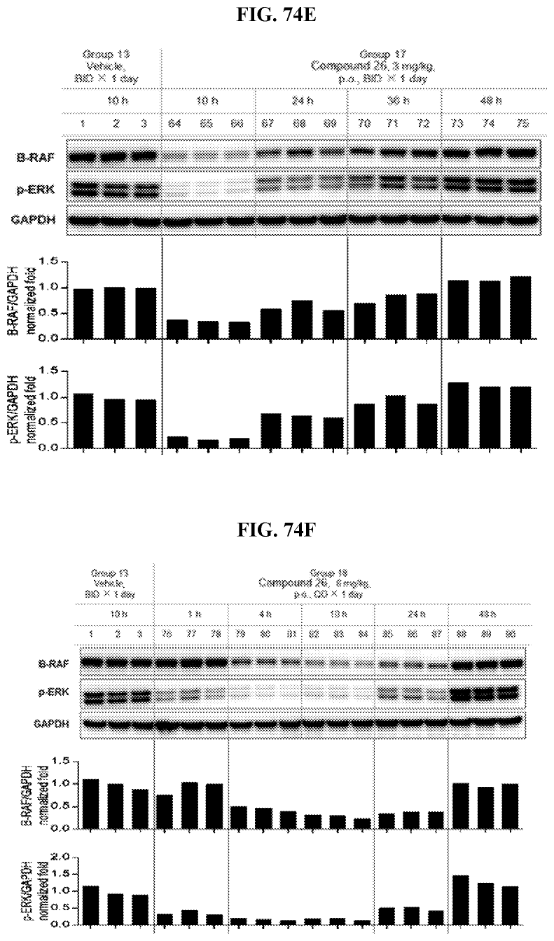

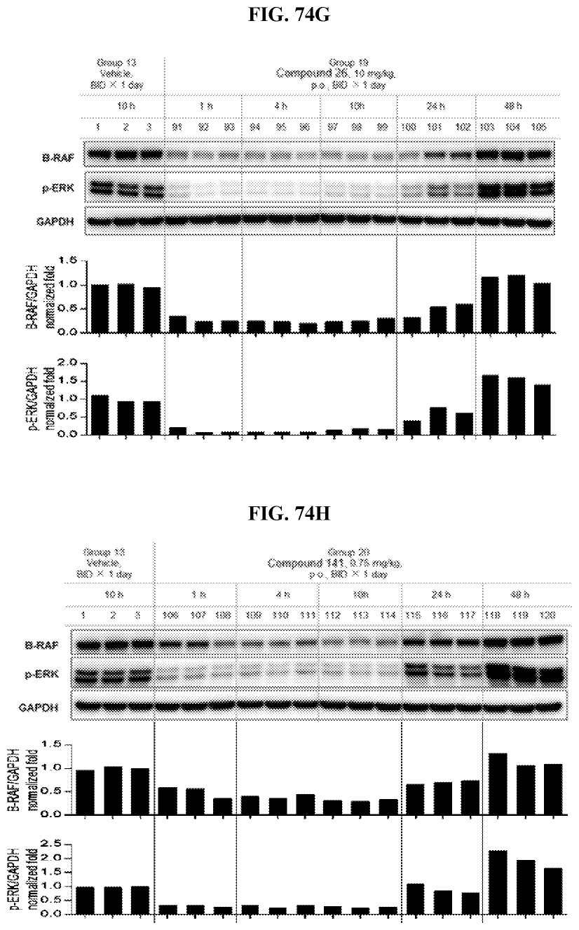

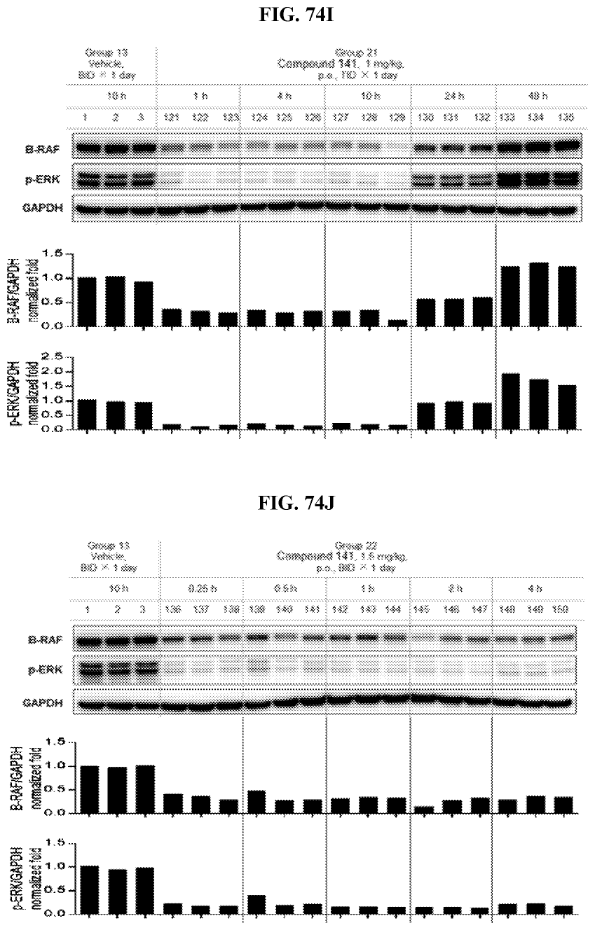

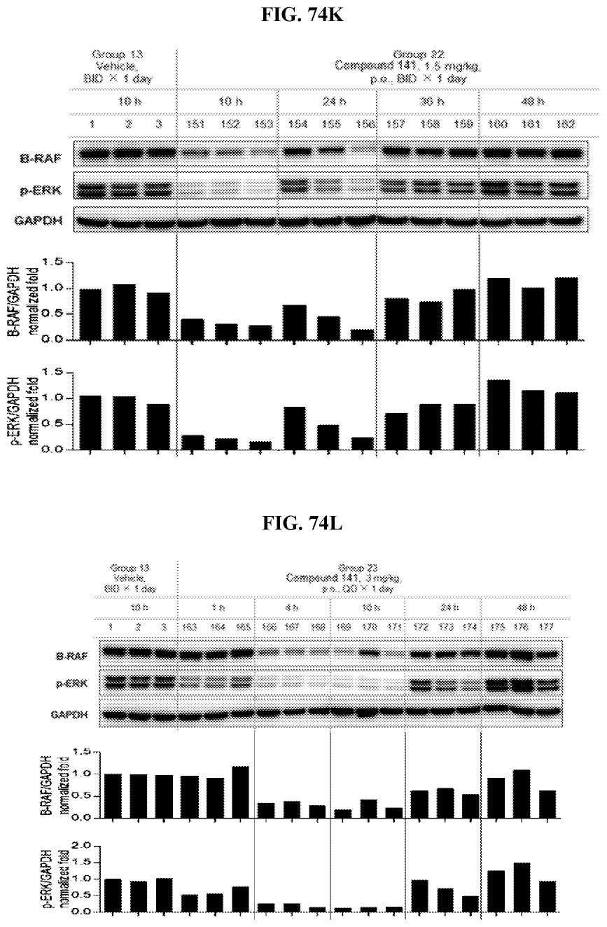

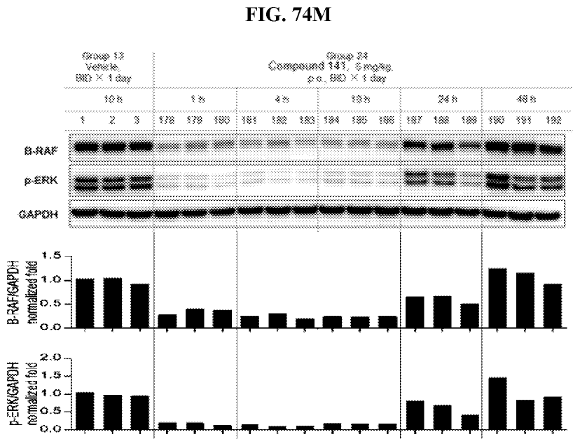

The results of BRAF and p-ERK expressions in A375 tumors are shown in FIG. 74A-74M.

### Example 14 In Vivo Therapeutic Efficacy of Compound 26 and Compound 141

### Method

Agents were tested in the A375 human melanoma xenografts. Compound 26 and Compound 141 were administered to BALB/c nude mice bearing A375 tumors from PG-DO and dosed via PO twice a day (BID) or third a day (TIP) or dosed P daily (QD) from PG-D. Dabrafenib was administered to BALB/c nude mice bearing A375 tumors from PG-D1 and dosed via P daily (QD). Compound 26 was dosed at 1.5 mg/kg (QD), 2 mg/kg (TID), 3 mg/kg (BID), 6 mg/kg (QD) and 10 mg/kg (BID) for 21 days. Compound 141 was dosed at 0.75 mg/kg (BID), 1 mg/kg (TID), 1.5 mg/kg (BID), 3 mg/kg (QD) and 5 mg/kg (BID) for 21 days. Dabrafenib was dosed at 100 mg/kg for 21 days. This study was non-GLP.

### Experimental Design

TABLE 75
Study Design for Efficacy Study

| | | | Weeks 1-3 | Dosing | Dosing | |
|---|---|---|---|---|---|---|
| Group | N^{a} | Treatment^{b} | Dose (mg/kg) | Route | Schedule^{c} | Schedule Post Dosing |
| 1 | 6 | Vehicle | — | p.o. | BID × 21 days | Monitor regrowth for 3 weeks |
| 2 | 6 | Dabrafenib | 100 | p.o. | QD × 21 days | Collection post last dose |
| | 6 | | | | | Monitor regrowth for 5 weeks |
| 3 | 6 | Compound 26 | 1.5 | p.o. | BID × 21 days | Collection post last dose |
| | 6 | | | | | Monitor regrowth for 5 weeks |
| 4 | 6 | Compound 26 | 2 | p.o. | TID × 21 days | Collection post last dose |
| | 6 | | | | | Monitor regrowth for 5 weeks |
| 5 | 6 | Compound 26 | 3 | p.o. | BID × 21 days | Collection post last dose |
| | 6 | | | | | Monitor regrowth for 5 weeks |
| 6 | 6 | Compound 26 | 6 | p.o. | QD × 21 days | Collection post last dose |
| | 6 | | | | | Monitor regrowth for 5 weeks |
| 7 | 6 | Compound 26 | 10 | p.o. | BID × 21 days | Collection post last dose |
| | 6 | | | | | Monitor regrowth for 5 weeks |
| 8 | 6 | Compound 141 | 0.75 | p.o. | BID × 21 days | Collection post last dose |
| | 6 | | | | | Monitor regrowth for 4 weeks |
| 9 | 6 | Compound 141 | 1 | p.o. | TID × 21 days | Collection post last dose |
| | 6 | | | | | Monitor regrowth for 5 weeks |
| 10 | 6 | Compound 141 | 1.5 | p.o. | BID × 21 days | Collection post last dose |
| | 6 | | | | | Monitor regrowth for 5 weeks |
| 11 | 6 | Compound 141 | 3 | p.o. | QD × 21 days | Collection post last dose |
| | 6 | | | | | Monitor regrowth for 5 weeks |
| 12 | 6 | Compound 141 | 5 | p.o. | BID × 21 days | Collection post last dose |
| | 6 | | | | | Monitor regrowth for 5 weeks |

Note:
^{a}N: animal number; Dosing volume: adjusted dosing volume based on body weight 10 μL/g.
^{b}The formulations for vehicle, Compound 26 and Compound 141 were 20% PEG400 + 80% (25% SBE-β-CD in ddH₂O). The formulation for dabrafenib was 10% PEG400 + 90% (11.1% HP-β-CD) in ddH²O.
^{c}Plasma and tissues samples were collected from 6 mice of groups 2-12 post last dose on PG-D21. The remaining mice from groups 1-12 were kept dosing-free regrowth monitoring. The mice were euthanized when individual mouse's tumor volume was over 3000 mm³.

TABLE 76
Sample Collection Design for Efficacy Study

| | | | | Timepoint | Timepoint | |
|---|---|---|---|---|---|---|
| | | Weeks 1-3 | | for Plasma | for Tissues | |
| Group | Treatment | Dose (mg/kg) | Animal ID | Collection^{a} | Collection^{b} | |
| 2 | Dabrafenib | 100 | #2-1, #2-2, #2-3 | 0.25 h, 1 h, 2 h, 4 h | 4 | h |
| | | | #2-4, #2-5, #2-6 | 0.5 h, 8 h, 10 h, 24 h | 24 | h |
| 3 | Compound | 1.5 | #3-1, #3-2, #3-3 | 0.25 h, 1 h, 2 h, 4 h | 4 | h |
| | 26 | | #3-4, #3-5, #3-6 | 0.5 h, 8 h, 10 h, 24 h | 24 | h |
| 4 | Compound | 2 | #4-1, #4-2, #4-3 | 0.25 h, 1 h, 2 h, 4 h | 4 | h |
| | 26 | | #4-4, #4-5, #4-6 | 0.5 h, 8 h, 10 h, 24 h | 24 | h |
| 5 | Compound | 3 | #5-1, #5-2, #5-3 | 0.25 h, 1 h, 2 h, 4 h | 4 | h |
| | 26 | | #5-4, #5-5, #5-6 | 0.5 h, 8 h, 10 h, 24 h | 24 | h |
| 6 | Compound | 6 | #6-1, #6-2, #6-3 | 0.25 h, 1 h, 2 h, 4 h | 4 | h |
| | 26 | | #6-4, #6-5, #6-6 | 0.5 h, 8 h, 10 h, 24 h | 24 | h |
| 7 | Compound | 10 | #7-1, #7-2, #7-3 | 0.25 h, 1 h, 2 h, 4 h | 4 | h |
| | 26 | | #7-4, #7-5, #7-6 | 0.5 h, 8 h, 10 h, 24 h | 24 | h |
| 8 | Compound | 0.75 | #8-1, #8-2, #8-3 | 0.25 h, 1 h, 2 h, 4 h | 4 | h |
| | 141 | | #8-4, #8-5, #8-6 | 0.5 h, 8 h, 10 h, 24 h | 24 | h |
| 9 | Compound | 1 | #9-1, #9-2, #9-3 | 0.25 h, 1 h, 2 h, 4 h | 4 | h |
| | 141 | | #9-4, #9-5, #9-6 | 0.5 h, 8 h, 10 h, 24 h | 24 | h |
| 10 | Compound | 1.5 | #10-1, #10-2, #10-3 | 0.25 h, 1 h, 2 h, 4 h | 4 | h |
| | 141 | | #10-4, #10-5, #10-6 | 0.5 h, 8 h, 10 h, 24 h | 24 | h |
| 11 | Compound | 3 | #11-1, #11-2, #11-3 | 0.25 h, 1 h, 2 h, 4 h | 4 | h |
| | 141 | | #11-4, #11-5, #11-6 | 0.5 h, 8 h, 10 h, 24 h | 24 | h |
| 12 | Compound | 5 | #12-1, #12-2, #12-3 | 0.25 h, 1 h, 2 h, 4 h | 4 | h |
| | 141 | | #12-4, #12-5, #12-6 | 0.5 h, 8 h, 10 h, 24 h | 24 | h |

Note:
^{a}Plasma samples were collected at 0.25, 0.5, 1, 2, 4, 8, 10 and 24 h post last dose for PK analysis.
^{b}Tissues (tumor, brain, liver, skin, spleen and lung) samples were collected at 4 and 24 h post last dose for PK.

### Materials

### Animals and Housing Condition

One hundred and thirty-eight female BALB/c nude Mus Musculus mice procured from Shanghai Lingchang Biological Science and Technology Co., LTD between the ages of six to eight weeks and with body weight between eighteen and twenty-two grams were used for the study. A spare mouse was also procured.

The mice were kept in individual ventilation cages at constant temperature and humidity with 6 animals in each cage. The cage was kept between approximately 20 to 26° C. with a humidity of 40-70%. The cages were made of polycarbonate with a size of 375 mm×215 mm×180 mm and corn cob bedding was changed twice per week.

The animals were fed irradiation sterilized dry granule food during the entire study period. The animals had free access to food and sterile drinking water. Cages were identified with the following information: number of animals, gender, strain, date received, treatment, study number, group number and the starting date of the treatment and each animal was marked by ear coding.

Test Articles

| | Molecular | Formula |
|---|---|---|
| Compound | Weight | Weight |
| Compound 26 | 911.33 | 911.99 |
| Compound 141 | 895.34 | 895.99 |
| Dabrafenib | 519.56 | 519.56 |

### Experimental Methods and Procedures

### Cell Culture

The A375 tumor cells were maintained in vitro in DMEM medium supplemented with 10% fetal bovine serum and 1% Penicillin-Streptomycin at 37° C. in an atmosphere of 5% CO₂ in air. The tumor cells were routinely sub-cultured twice weekly. The cells growing in an exponential growth phase were harvested and counted for tumor inoculation. The details of reagents are shown in Table 77 below.

TABLE 77
Vendor and Cat No. of Cell Line and Reagents

| Cell Line/Reagent | Vendor | Catalog # |
|---|---|---|
| A375 | ATCC | CRL-1619 |
| DMEM | Gibco | 11995-065 |
| Penicillin-Streptomycin | Gibco | 15240-062 |
| 0.25% Trypsin-EDTA | Gibco | 25200-072 |
| FBS | Corning | 35-081-CV |
| DPBS | Corning | 21-031-CVC |
| Matrigel | Corning | 354234 |

### Tumor Inoculation

Each mouse was inoculated subcutaneously at the right flank with A375 cells (5×10⁶) in 0.2 mL of PBS supplemented with Matrigel (PBS: Matrigel=1:1) for tumor development. Animals were grouped for efficacy study on day 15 after tumor inoculation when the average tumor volume reached 220 mm³. The test articles administration and the animal numbers in each group are shown in the above experimental Table 75.

### Reagents Used in the Study

TABLE 78
Vendor and Cat No. of Reagents Used for Formulation Preparation

| Reagent | Vendor | Catalog # |
|---|---|---|
| PEG400 | Sigma | P3265 |
| SBE-β-CD | Kunshan Ruisike | 10059859 |
| | Chemical Materials | |
| Tween 80 | Sigma | P4780 |
| HPMC | Sigma | S25213 |
| HP-β-CD | Energy chemical | E080016 |

### Vehicle Preparation

### Preparation of 0.5% HPMC+0.2% Tween 80 in ddH₂O

HPMC (1.00 g) was accurately weighed into a 500 mL glass bottle. Then, 160 mL ddH₂O and 0.400 mL Tween 80 were added to the bottle and the mixture was stirred to obtain a clear solution. Then ddH₂O was added to a total volume of 200 mL and the mixture was mixed well. The mixture was stirred overnight until a clear solution was formed.

### Preparation of 11.1% HP-β-CD in ddH₂O

HP-β-CD (22.2 g) was accurately weighed into a 500 mL glass bottle. Then, 150 mL ddH₂O was added to the bottle and the mixture was stirred to obtain a clear solution. Then ddH₂O was added to a total volume of 200 mL and the mixture was mixed well.

### Preparation of 25% SBE-β-CD in ddH₂O

SBE-β-CD (50 g) was accurately weighed into a 500 mL glass bottle. Then, 150 mL ddH₂O was added to the bottle and the mixture was stirred to obtain a clear solution. Then ddH₂O was added to a total volume of 200 mL and the mixture was mixed well.

### Testing Article Formulation Preparation

TABLE 79
Formulation of Dabrafenib Preparation

| | | | | Volume of | Volume of 11.1% | |
|---|---|---|---|---|---|---|
| | | Conc. of | Compound | PEG400 to | HP-β-CD in ddH₂O | |
| Compound^{a} | Dose | solution | amount | be added | to be added | Dosing Solution |
| ID | (mg/kg) | (mg/mL) | (mg) | (mL) (10%) | (mL) (90%) | observations |
| Dabrafenib | 100 | 10 | 101.09 | 1.008 | 9.072 | Suspension |

Note:
^{a}The formulation for Dabrafenib was prepared once every three days.

TABLE 80
Formulation of Compound 26 and Compound 141 Preparation

| | | | | Volume of | Volume of 25% | |
|---|---|---|---|---|---|---|
| | | Conc. of | Compound | PEG400 to be | SBE-β-CD to be | Dosing |
| Compound^{a} | Dose | solution | amount | added (mL) | added (mL) | Solution |
| ID | (mg/kg) | (mg/mL) | (mg) | (20%) | (80%) | observations |
| Compound 26 | 1.5 | 0.15 | 3.29 | 4.326 | 17.304 | clear solution |
| Compound 26 | 2 | 0.2 | 6.45 | 6.361 | 25.444 | clear solution |
| Compound 26 | 3 | 0.3 | 5.90 | 3.879 | 15.516 | clear solution |
| Compound 26 | 6 | 0.6 | 6.38 | 2.097 | 8.389 | clear solution |
| Compound 26 | 10 | 1 | 19.55 | 3.856 | 15.424 | clear solution |
| Compound 141 | 0.75 | 0.075 | 1.49 | 3.924 | 15.694 | clear solution |
| Compound 141 | 1 | 0.1 | 3.48 | 6.873 | 27.492 | clear solution |
| Compound 141 | 1.5 | 0.15 | 3.22 | 4.240 | 16.958 | clear solution |
| Compound 141 | 3 | 0.3 | 3.20 | 2.107 | 8.427 | clear solution |
| Compound 141 | 5 | 0.5 | 10.45 | 4.128 | 16.511 | clear solution |

Note:
^{a}The formulations for Compound 26 and Compound 141 were prepared once every three days.

### Observations

At the time of routine monitoring, the animals were checked daily for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset. Animals that were observed to be in a continuing deteriorating condition or their tumor size exceeding 3,000 mm³ were euthanized prior to death or before reaching a comatose state.

### Tumor Measurements and the Endpoints

Prior to the onset of drug treatment, mice were measured for tumor size in two dimensions using a caliper, and the tumor volume (mm³) was calculated using formula V=0.5 a×b² where a and b are the long and short diameters of the tumor in mm, respectively. Mice were randomized into different treatment groups based on the tumor volume.

The major endpoint was to see if the tumor growth could be delayed or mice could be cured. The tumor size was then used for calculations of T/C value. The T/C value (in percent) is an indication of antitumor effectiveness; T and C are the mean volumes of the treated and control groups, respectively on specified day.

TGI was calculated for each group using the formula: TGI (%)=[1−(Tᵢ−T₀)/(Cᵢ−C₀)]×100; Tᵢ is the average tumor volume of a treatment group on a given day, T₀ is the average tumor volume of the treatment group on the day of treatment start, Cᵢ is the average tumor volume of the vehicle control group on the same day with Tᵢ, and C₀ is the average tumor volume of the vehicle group on the day of treatment start.

### Sample Collection

### Plasma Sample Preparation

The plasma samples were collected at predetermined time points for PK analysis. For non-termination plasma collection: the blood samples were collected into the 1.5 mL Eppendorf tubes containing 3 μL of 0.5 M EDTA-K2. And then the anticoagulant blood samples were centrifuged at 2,500 g at 4° C. for 15 minutes. For termination plasma collection: the mice was euthanized at predetermined time points post the treatment, the blood samples were collected into the 1.5 mL Eppendorf tubes containing 15 μL of 0.5 M EDTA-K2. And then the anticoagulant blood samples were centrifuged at 2,500 g at 4° C. for 15 minutes. The plasma samples were separated into 2 aliquots. The ₁^{st} was used for PK analysis, and the 2^{nd} was served as backup.

### Tumor Sample Preparation

The tumor samples were collected at predetermined time points for PK and WB analysis. The tumor was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Brain Sample Preparation

The brain samples were collected at predetermined time points for PK and potential WB analysis. The brain was dissected out and split into 3 parts (left brain, right brain upper and right brain lower), the left brain was used for PK, the right brain upper was used for WB and the last one was used as the backup for the study. The samples were transferred into pre-labeled tubes and quickly froze in liquid nitrogen within 2 minutes after dissection. The samples were stored at −80° C. before analysis.

### Liver Sample Preparation

Liver samples were collected at predetermined time points for potential PK and WB analysis. The liver was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Lung Sample Preparation

The tumor samples were collected at predetermined time points for potential PK and WB analysis. The lung was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Spleen Sample Preparation

The spleen samples were collected at predetermined time points for potential PK and WB analysis. The spleen was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Skin Sample Preparation

The skin samples were collected at predetermined time points for potential PK and WB analysis. The mice back skin was dissected out and split into 3 parts. The 1^{st} part was used for PK analysis, the 2^{nd} part was used for WB analysis and the last one was used as backup for the study. The samples were transferred into pre-labeled tubes and quickly froze in liquid nitrogen within 2 minutes after dissection. The samples were stored at −80° C. before analysis.

### Pharmacokinetics Assay

The harvested plasma and tumor samples were processed for pharmacokinetics analysis by LC-MS/MS with a concentration range of 1.00-1,000 ng/mL for dabrafenib in plasma and 0.0500-50 ng/mL in tumor and brain homogenate, 5.00-10,000 ng/mL for Compound 141 in plasma and 0.200~200 ng/ml in tumor and brain homogenate. 20.0~20,000 ng/mL for Compound 26 in plasma and 0.5~500 ng/mL for Compound 26 tumor and brain homogenate.

### Analytical Method for Dabrafenib, Compound 26 and Compound 141 in Plasma, Tumor and Brain Homogenate

Dabrafenib, Compound 26 and Compound 141 in female BALB/c nude mice plasma (K2-EDTA), tumor and brain homogenate were analyzed by LC-MS/MS (Triple Quad 6500+ Low Mass). Verapamil (5 ng/mL) in acetonitrile: methanol (V: V=95:5) was used as the internal standard for Dabrafenib, Compound 26 and Compound 141 in plasma. Verapamil (0.5 ng/mL) in acetonitrile: methanol (V: V=95:5) was used as the internal standard for Dabrafenib, Compound 26 and Compound 141 in tumor and brain homogenate.

MS condition: ESI positive, MRM detection, Dabrafenib: [M+H]⁺ 519.95/307.00 m/z; Compound 26: [M+H]⁺ 913.31/854.20 m/z; Compound 141: [M+H]⁺ 896.20/837.30 m/z; Verapamil (IS): [M+H]⁺ 455.23/165.10 m/z;

LC conditions for Compound 141 in plasma, tumor and brain
homogenate: mobile phase A: 5 mM HCOONH₄ in H₂O:ACN (V:V =
95:5); mobile phase B: 5 mM HCOONH₄ in ACN:H₂O (V:V =
95:5); injection: 1 μL for plasma, 10 μL for tumor
and brain; Column: Waters ACQUITY BEH C18 1.7 μm 2.1*50
mm; Column temperature: 40.0° C.; Flow rate: 0.6 mL/min.

| Time (min) | Mobile Phase B (%) |
|---|---|
| Initial | 20 |
| 0.10 | 20 |
| 1.20 | 95 |
| 1.40 | 95 |
| 1.41 | 20 |
| 1.60 | 20 |

LC conditions for Dabrafenib and Compound 26 in plasma,
tumor and brain homogenate: mobile phase A: 5 mM HCOONH₄ in
H₂O:ACN (V:V = 95:5); mobile phase B: 5 mM HCOONH₄ in
ACN:H₂O (V:V = 95:5); injection: 1 μL for Dabrafenib
in plasma, 5 μL for Dabrafenib in tumor and brain; 5
μL for Compound 26 in plasma; 10 μL for Compound
26 in tumor and brain; Column: Waters ACQUITY BEH C18 1.7
μm 2.1*50 mm; Column temperature: 40.0° C.; Flow rate: 0.6 mL/min.

| Time (min) | Mobile Phase B (%) |
|---|---|
| Initial | 30 |
| 0.10 | 30 |
| 1.10 | 90 |
| 1.30 | 90 |
| 1.31 | 30 |
| 1.50 | 30 |

Retention time: Dabrafenib: 1.02 min; Compound 141: 0.83 min; Compound 26: 0.68 min; Verapamil(IS) for Dabrafenib: 0.78 min; Verapamil(IS) for Compound 141: 0.89 min; Verapamil(IS) for Compound 26: 0.76 min.

### Preparation of Plasma and Tumor Homogenate Samples

An aliquot of 10 μL unknown sample, calibration standard, quality control, dilute quality control, single blank and double blank samples were added to the 96-well plate. Each sample for Dabrafenib, Compound 26 and Compound 141 in plasma (except the double blank) was quenched with 200 μL IS respectively (double blank sample was quenched with 200 μL ACN: MeOH (V: V=95:5)); each sample for Dabrafenib, Compound 26 and Compound 141 in tumor and brain(except the double blank) was quenched with 100 μL IS respectively (double blank sample was quenched with 100 μL ACN: MeOH (V: V=95:5)); and then the mixture was vortex-mixed for 5 min at 800 rpm and centrifuged for 15 min at 3220 g (4000 rpm), 4° C. Then 40 μL supernatant was diluted with 40 μL of H₂O for Dabrafenib, Compound 26 and Compound 141 in plasma, tumor and brain homogenate, the diluted solution was directly injected for LC-MS/MS analysis.

Dilution procedure: When dilution factor is 50, an aliquot of 2 μL sample was added with 8 μL blank matrix, and then an aliquot of 2 μL diluted sample was added with 18 μL blank matrix. When the dilution factor is 10, an aliquot of 2 μL sample was added with 18 μL blank matrix.

Calibration curve: 1.00-1000 ng/mL for Dabrafenib in plasma; 5.00-10000 ng/mL for Compound 141 in plasma; 20.0-20000 ng/mL for Compound 26 in plasma; 0.0500-50 ng/mL for Dabrafenib in tumor and brain; 0.200-200 ng/mL for Compound 141 in tumor and brain; 0.500-500 ng/mL for Compound 26 in tumor and brain.

### Data Management and Statistical Analyses

A one-way ANOVA followed by Dunnett's multiple comparison test was performed to compare tumor volume among vehicle group (Group 1) and treatment groups (Group 2-Group 12). All data were analyzed using GraphPad Prism, ns indicates p>0.05, * indicates p<0.05, ** indicates p<0.01, *** indicates p<0.001 and **** indicates p<0.0001.

### Results

### Body Weight Change

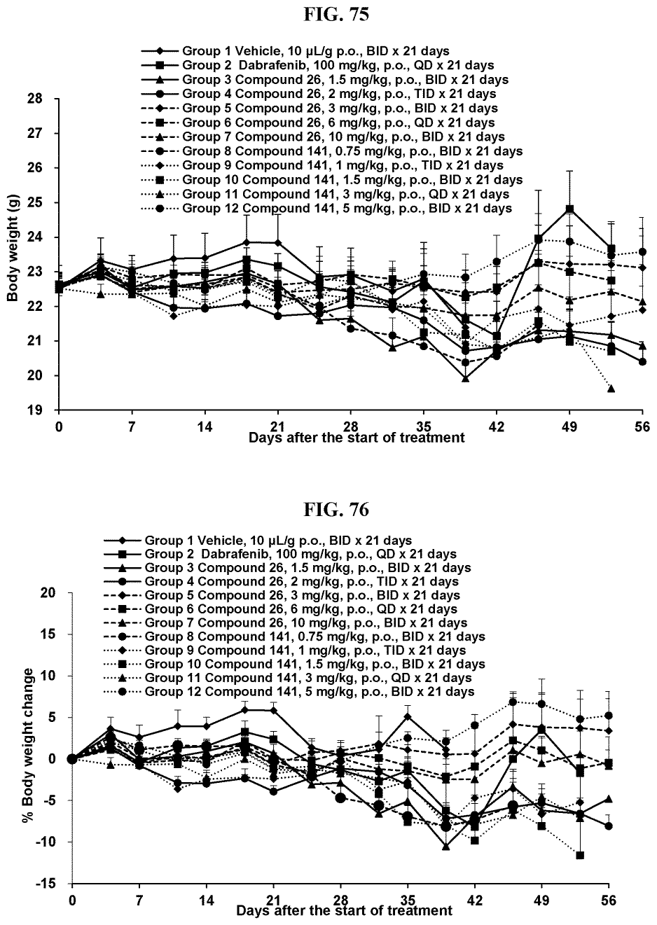

Animal body weight was monitored regularly as an indirect measurement of toxicity. Body weight changes after administration of Compound 26, Compound 141 and dabrafenib are shown in FIG. 75 (Data points represent Group Mean Body Weight. Error bars represent Standard Error of the Mean (SEM)) and FIG. 76 (Data points represent Percent Group Mean Change in BW. Error bars represent Standard Error of the Mean (SEM)).

### Tumor Growth Curve

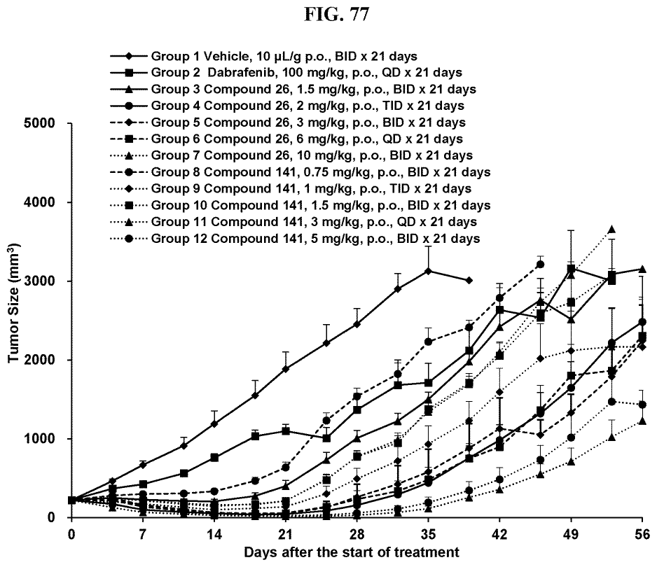

Tumor growth curves are shown in FIG. 77. Data points represent Group Mean Tumor Volume. Error bars represent Standard Error of the Mean (SEM).

### Tumor Volume Trace

Mean tumor size over time in female BALB/c nude mice bearing A375 human melanoma xenobrafts is shown in Table 81.

TABLE 81
Tumor Volume Trace Over Time (mm³)

Days

| Group# | 0 | 4 | 7 | 11 | 14 | 18 | 21^{b} | 25 | 28 |
|---|---|---|---|---|---|---|---|---|---|
| Group 1, | 220 ± 22^{a} | 464 ± 24 | 668 ± 51 | 910 ± 106 | 1189 ± 164 | 1548 ± 192 | 1884 ± 217 | 2213 ± 233 | 2453 ± 201 |
| Vehicle, | | | | | | | | | |
| p.o., BID × 21 days | | | | | | | | | |
| Group 2, | 220 ± 15 | 367 ± 24 | 421 ± 28 | 560 ± 38 | 756 ± 54 | 1031 ± 79 | 1095 ± 86 | 1007 ± 137 | 1366 ± 209 |
| Dabrafenib, 100 mg/kg, | | | | | | | | | |
| p.o., QD × 21 days | | | | | | | | | |
| Group 3, | 220 ± 16 | 249 ± 21 | 227 ± 23 | 210 ± 26 | 204 ± 26 | 271 ± 44 | 400 ± 70 | 730 ± 94 | 1006 ± 99 |
| Compound 26, 1.5 mg/kg, | | | | | | | | | |
| p.o., BID × 21 days | | | | | | | | | |
| Group 4, | 220 ± 15 | 172 ± 13 | 99 ± 9 | 68 ± 8 | 51 ± 7 | 36 ± 4 | 41 ± 5 | 85 ± 16 | 156 ± 32 |
| Compound 26, 2 mg/kg, | | | | | | | | | |
| p.o., TID × 21 days | | | | | | | | | |
| Group 5, | 220 ± 15 | 219 ± 16 | 136 ± 11 | 83 ± 6 | 52 ± 5 | 45 ± 7 | 48 ± 9 | 129 ± 75 | 258 ± 165 |
| Compound 26, 3 mg/kg, | | | | | | | | | |
| p.o., BID × 21 days | | | | | | | | | |
| Group 6, | 220 ± 16 | 243 ± 16 | 158 ± 10 | 101 ± 7 | 62 ± 4 | 49 ± 7 | 54 ± 13 | 137 ± 74 | 230 ± 109 |
| Compound 26, 6 mg/kg, | | | | | | | | | |
| p.o., QD × 21 days | | | | | | | | | |
| Group 7, | 220 ± 15 | 126 ± 10 | 65 ± 6 | 42 ± 4 | 26 ± 3 | 20 ± 2 | 17 ± 2 | 17 ± 2 | 29 ± 3 |
| Compound 26, 10 mg/kg, | | | | | | | | | |
| p.o., BID × 21 days | | | | | | | | | |
| Group 8, | 220 ± 16 | 279 ± 20 | 299 ± 25 | 305 ± 27 | 333 ± 29 | 465 ± 48 | 634 ± 67 | 1230 ± 98 | 1536 ± 103 |
| Compound 141, 0.75 mg/kg, | | | | | | | | | |
| p.o., BID × 21 days | | | | | | | | | |
| Group 9, | 220 ± 16 | 199 ± 14 | 174 ± 12 | 131 ± 11 | 99 ± 11 | 117 ± 18 | 138 ± 22 | 303 ± 94 | 492 ± 132 |
| Compound 141, 1 mg/kg, | | | | | | | | | |
| p.o., TID × 21 days | | | | | | | | | |
| Group 10, | 220 ± 15 | 245 ± 18 | 213 ± 17 | 180 ± 13 | 159 ± 13 | 166 ± 14 | 208 ± 21 | 476 ± 63 | 773 ± 76 |
| Compound 141, 1.5 mg/kg, | | | | | | | | | |
| p.o., BID × 21 days | | | | | | | | | |
| Group 11, | 220 ± 15 | 253 ± 15 | 214 ± 17 | 169 ± 15 | 144 ± 16 | 172 ± 19 | 208 ± 30 | 486 ± 63 | 773 ± 63 |
| Compound 141, 3 mg/kg, | | | | | | | | | |
| p.o., QD × 21 days | | | | | | | | | |
| Group 12, | 220 ± 15 | 166 ± 11 | 100 ± 7 | 60 ± 4 | 38 ± 2 | 27 ± 3 | 23 ± 3 | 31 ± 9 | 56 ± 24 |
| Compound 141, 5 mg/kg, | | | | | | | | | |
| p.o., BID × 21 days | | | | | | | | | |

| | Days | | | | | | | |
|---|---|---|---|---|---|---|---|---|
| Group# | 32 | 35 | 39 | 42 | 46 | 49 | 53 | 56 |
| Group 1, | 2899 ± 196 | 3127 ± 314 | 3009 | — | — | — | — | — |
| Vehicle, | | | | | | | | |
| p.o., BID × 21 days | | | | | | | | |
| Group 2, | 1679 ± 323 | 1711 ± 245 | 2116 ± 279 | 2634 ± 337 | 2535 ± 321 | 3163 ± 478 | 3001 | — |
| Dabrafenib, 100 mg/kg, | | | | | | | | |
| p.o., QD × 21 days | | | | | | | | |
| Group 3, | 1224 ± 99 | 1497 ± 94 | 1978 ± 148 | 2420 ± 205 | 2759 ± 274 | 2514 ± 268 | 3087 ± 444 | 3153 |
| Compound 26, 1.5 mg/kg, | | | | | | | | |
| p.o., BID x 21 days | | | | | | | | |
| Group 4, | 292 ± 57 | 441 ± 86 | 754 ± 156 | 980 ± 198 | 1320 ± 265 | 1646 ± 332 | 2216 ± 435 | 2482 ± 579 |
| Compound 26, 2 mg/kg, | | | | | | | | |
| p.o., TID × 21 days | | | | | | | | |
| Group 5, | 423 ± 234 | 578 ± 289 | 878 ± 311 | 1129 ± 390 | 1050 ± 191 | 1328 ± 237 | 1789 ± 341 | 2242 ± 453 |
| Compound 26, 3 mg/kg, | | | | | | | | |
| p.o., BID x 21 days | | | | | | | | |
| Group 6, | 335 ± 129 | 478 ± 161 | 751 ± 185 | 894 ± 208 | 1363 ± 315 | 1802 ± 396 | 1863 ± 377 | 2305 ± 461 |
| Compound 26, 6 mg/kg, | | | | | | | | |
| p.o., QD x 21 days | | | | | | | | |
| Group 7, | 62 ± 16 | 112 ± 34 | 253 ± 70 | 357 ± 91 | 550 ± 131 | 711 ± 168 | 1021 ± 215 | 1128 ± 250 |
| Compound 26, 10 mg/kg, | | | | | | | | |
| p.o., BID x 21 days | | | | | | | | |
| Group 8, | 1821 ± 140 | 2230 ± 178 | 2413 ± 86 | 2786 ± 127 | 3213 ± 101 | — | — | — |
| Compound 141, 0.75 mg/kg, | | | | | | | | |
| p.o., BID x 21 days | | | | | | | | |
| Group 9, | 718 ± 197 | 933 ± 232 | 1233 ± 240 | 1591 ± 302 | 2017 ± 443 | 2117 ± 505 | 2166 ± 495 | 2162 ± 633 |
| Compound 141, 1 mg/kg, | | | | | | | | |
| p.o., TID × 21 days | | | | | | | | |
| Group 10, | 946 ± 93 | 1374 ± 116 | 1709 ± 117 | 2051 ± 177 | 2593 ± 258 | 2727 ± 382 | 3079 ± 78 | — |
| Compound 141, 1.5 mg/kg, | | | | | | | | |
| p.o., BID x 21 days | | | | | | | | |
| Group 11, | 985 ± 85 | 1339 ± 71 | 1690 ± 103 | 2099 ± 177 | 2737 ± 176 | 3078 ± 168 | 3659 | — |
| Compound 141, 3 mg/kg, | | | | | | | | |
| p.o., QD x 21 days | | | | | | | | |
| Group 12, | 109 ± 40 | 190 ± 67 | 345 ± 111 | 484 ± 147 | 732 ± 181 | 1015 ± 308 | 1470 ± 351 | 1433 ± 181 |
| Compound 141, 5 mg/kg, | | | | | | | | |
| p.o., BID x 21 days | | | | | | | | |

Note:
^{a}Data are shown as Mean ± SEM.
^{b}Plasma and tissues samples were collected from 6 mice of groups 2-12 post last dose on PG-D21. The remaining mice from groups 1-12 were kept dosing-free regrowth monitoring. The mice were euthanized when the individual mouse's tumor volume was over 3000 mm³.

### Tumor Growth Inhibition Analysis

Tumor growth inhibition of Compound 26, Compound 141 and dabrafenib in the treatment of female BALB/c nude mice bearing A375 xenografts was calculated based on tumor volume measured on PG-D321. TGI and T/C were calculated respectively and the data are detailed in Table 82.

TABLE 82
Tumor Growth Inhibition Analysis (T/C and TGI)^{a}

| | Tumor Size | | | |
|---|---|---|---|---|
| | (mm³)^{a} | | | |
| Treatment | on PG-D21 | T/C^{b} (%) | TGI^{c} (%) | p value^{d} |
| Group 1, | 1884 ± 217 | / | / | / |
| Vehicle, p.o., | | | | |
| BID × 28 days | | | | |
| Group 2, | 1095 ± 86 | 58.1 | 47.4 | **** |
| Dabrafenib, 100 mg/kg, | | | | |
| p.o., QD × 21 days | | | | |
| Group 3, | 400 ± 70 | 21.2 | 89.2 | **** |
| Compound 26, 1.5 mg/kg, | | | | |
| p.o., BID × 21 days | | | | |
| Group 4, | 41 ± 5 | 2.2 | 110.8 | **** |
| Compound 26, 2 mg/kg, | | | | |
| p.o., TID × 21 days | | | | |
| Group 5, | 48 ± 9 | 2.5 | 110.4 | **** |
| Compound 26, 3 mg/kg, | | | | |
| p.o., BID × 21 days | | | | |
| Group 6, | 54 ± 13 | 2.9 | 110.0 | **** |
| Compound 26, 6 mg/kg, | | | | |
| p.o., QD × 21 days | | | | |
| Group 7, | 17 ± 2 | 0.9 | 112.2 | **** |
| Compound 26, 10 mg/kg, | | | | |
| p.o., BID × 21 days | | | | |
| Group 8, | 634 ± 67 | 33.7 | 75.1 | **** |
| Compound 141, 0.75 mg/kg, | | | | |
| p.o., BID × 21 days | | | | |
| Group 9, | 138 ± 22 | 7.3 | 104.9 | **** |
| Compound 141, 1 mg/kg, | | | | |
| p.o., TID × 21 days | | | | |
| Group 10, | 208 ± 21 | 11.1 | 100.7 | **** |
| Compound 141, 1.5 mg/kg, | | | | |
| p.o., BID × 21 days | | | | |
| Group 11, | 208 ± 30 | 11.0 | 100.8 | **** |
| Compound 141, 3 mg/kg, | | | | |
| p.o., QD × 21 days | | | | |
| Group 12, | 23 ± 3 | 1.2 | 111.8 | **** |
| Compound 141, 5 mg/kg, | | | | |
| p.o., BID × 21 days | | | | |

Note:
^{a}Data are shown as Mean ± SEM.
^{b}Antitumor activity (T/C) was determined by dividing the average tumor volume for treated group (T) by the average tumor volume for control group (C).
^{c}Tumor Growth Inhibition (TGI) was calculated using the formula: TGI (%) = (1 − (T − T₀)/(C − C₀))*100, where T = mean tumor size at the terminal end of treatment group; T₀ = mean tumor size at day 0 of treatment group; C = mean tumor size at the terminal end of control group; and C₀ = mean tumor size at day 0 of control group.
^{d}A one-way ANOVA followed by Dunnett's multiple comparison test was performed to compare tumor volume among vehicle group and treatment groups. All data were analyzed using GraphPad Prism, **** indicates p < 0.0001.

### Pharmacokinetics Results

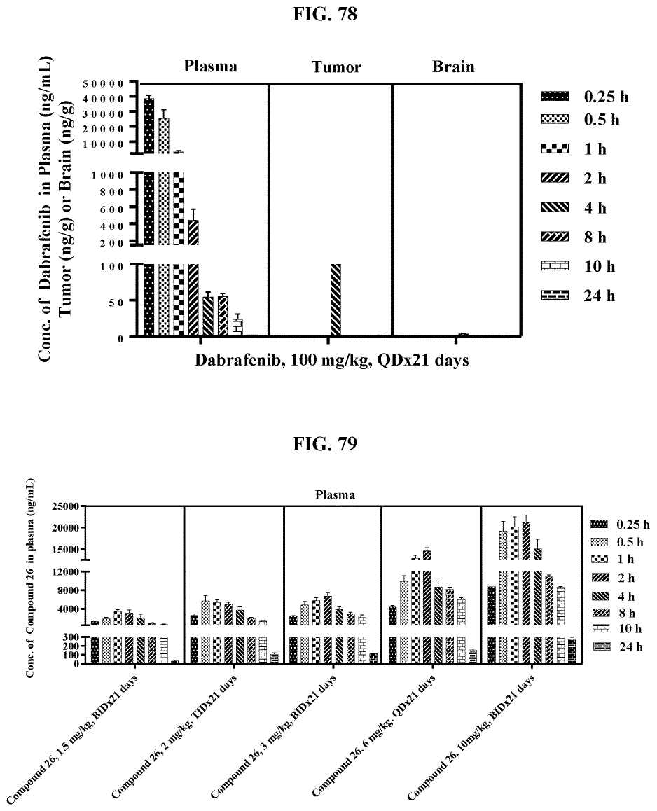

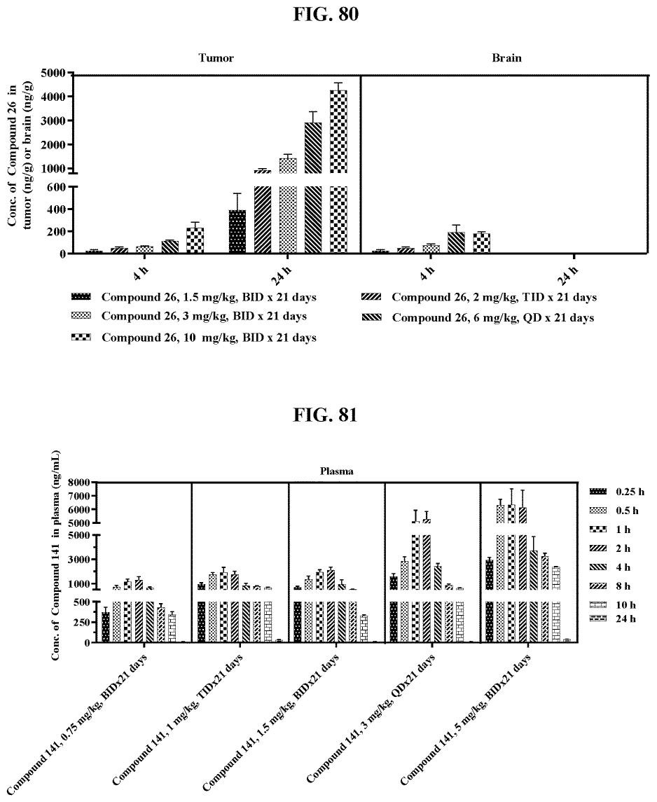

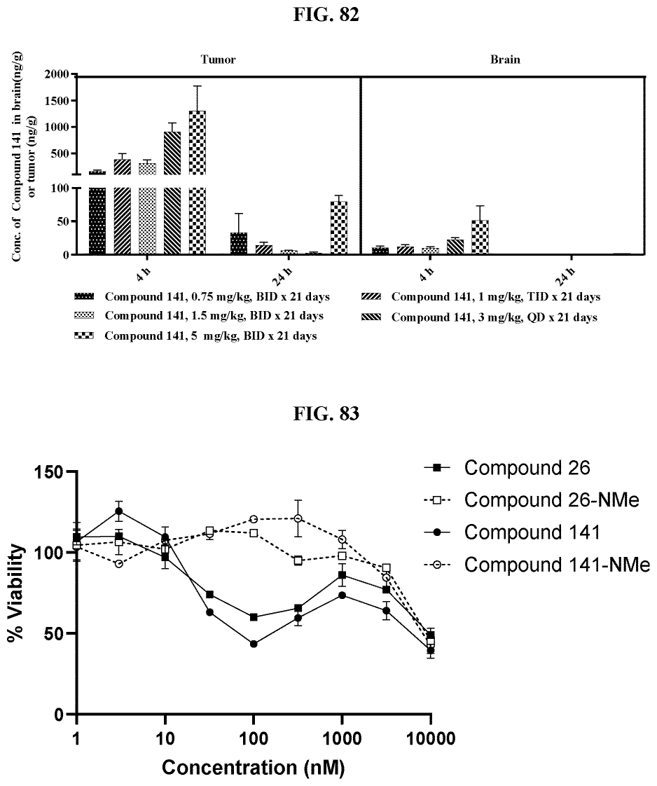

The concentrations of Compound 26, Compound 141 and dabrafenib in the treatment of female BALB/c nude mice bearing A375 xenografts are shown in Table 83 and FIG. 78-FIG. 82. Plasma AUC results at steady state are shown in Table 84.

TABLE 83
Mean Concentration of Test Articles in Plasma, Tumor and Brain

| | | | | Concentration | Concentration | Concentration |
|---|---|---|---|---|---|---|
| | Sample | | | in Plasma | in Tumor | in Brain |
| Group | time | | Analyte | (ng/mL) ± SEM | (ng/g) ± SEM | (ng/g) ± SEM |
| Group 2, | 0.25 | h | Dabrafenib | 38667 ± 2114 | — | — |
| Dabrafenib, | 0.5 | h | | 25667 ± 5687 | — | — |
| 100 mg/kg, | 1 | h | | 3267 ± 443 | — | — |
| p.o., QD × | 2 | h | | 444 ± 129 | — | — |
| 21 days | 4 | h | | 54.4 ± 7.02 | 137 ± 7.51 | 3.59 ± 0.558 |
| | 8 | h | | 55.4 ± 4.19 | — | — |
| | 10 | h | | 24.0 ± 6.86 | — | — |
| | 24 | h | | 2.27 | 1.43 ± 0.191 | BQL^{a} |
| Group 3, | 0.25 | h | Compound | 1327 ± 120 | — | — |
| Compound 26, | 0.5 | h | 26 | 1913 ± 332 | — | — |
| 1.5 mg/kg, | 1 | h | | 3493 ± 364 | — | — |
| p.o., BID × | 2 | h | | 3137 ± 671 | — | — |
| 21 days | 4 | h | | 2165 ± 716 | 392 ± 150 | 28.0 ± 8.47 |
| | 8 | h | | 912 ± 118 | — | — |
| | 10 | h | | 694 ± 79.9 | — | — |
| | 24 | h | | 32.3 | 11.8 ± 1.59 | BQL |
| Group 4, | 0.25 | h | Compound | 2630 ± 273 | — | — |
| Compound 26, | 0.5 | h | 26 | 5747 ± 1100 | — | — |
| 2 mg/kg, | 1 | h | | 5423 ± 571 | — | — |
| p.o., TID × | 2 | h | | 5107 ± 286 | — | — |
| 21 days | 4 | h | | 3790 ± 666 | 930 ± 57.3 | 51.1 ± 11.5 |
| | 8 | h | | 2063 ± 150 | — | — |
| | 10 | h | | 1547 ± 37.1 | — | — |
| | 24 | h | | 106 ± 17.7 | 80.7 ± 25.2 | BQL |
| Group 5, | 0.25 | h | Compound | 2487 ± 107 | — | — |
| Compound 26, | 0.5 | h | 26 | 4857 ± 801 | — | — |
| 3 mg/kg, | 1 | h | | 5833 ± 591 | — | — |
| p.o., BID × | 2 | h | | 6747 ± 675 | — | — |
| 21 days | 4 | h | | 3967 ± 452 | 1427 ± 159 | 66.6 ± 4.67 |
| | 8 | h | | 3027 ± 252 | — | — |
| | 10 | h | | 2473 ± 266 | — | — |
| | 24 | h | | 116 ± 2.40 | 54.3 ± 8.91 | BQL |
| Group 6, | 0.25 | h | Compound | 4437 ± 284 | — | — |
| Compound 26, | 0.5 | h | 26 | 9987 ± 1164 | — | — |
| 6 mg/kg, | 1 | h | | 12867 ± 669 | — | — |
| p.o., QD × | 2 | h | | 14600 ± 723 | — | — |
| 21 days | 4 | h | | 8710 ± 1875 | 2907 ± 459 | 116 ± 8.11 |
| | 8 | h | | 8223 ± 421 | — | — |
| | 10 | h | | 6203 ± 192 | — | — |
| | 24 | h | | 156 ± 10.9 | 338 ± 169 | 7.19 |
| Group 7, | 0.25 | h | Compound | 8793 ± 316.3 | — | — |
| Compound 26, | 0.5 | h | 26 | 19267 ± 2217 | — | — |
| 10 mg/kg, | 1 | h | | 20200 ± 2237 | — | — |
| p.o., BID × | 2 | h | | 21333 ± 1556 | — | — |
| 21 days | 4 | h | | 15133 ± 2206 | 4253 ± 313 | 233 ± 48.4 |
| | 8 | h | | 10900 ± 378.6 | — | — |
| | 10 | h | | 8660 ± 186 | — | — |
| | 24 | h | | 273 ± 24.8 | 663 ± 153 | 7.79 |
| Group 8, | 0.25 | h | Compound | 369 ± 64.7 | — | — |
| Compound 141, | 0.5 | h | 141 | 732 ± 139 | — | — |
| 0.75 mg/kg, | 1 | h | | 1144 ± 226 | — | — |
| p.o., BID × | 2 | h | | 1284 ± 277 | — | — |
| 21 days | 4 | h | | 663 ± 79.0 | 160 ± 24.9 | 10.7 ± 2.04 |
| | 8 | h | | 432 ± 45.3 | — | — |
| | 10 | h | | 343 ± 34.9 | — | — |
| | 24 | h | | 10.8 ± 2.44 | 33.0 | BQL |
| Group 9, | 0.25 | h | Compound | 950 ± 130 | — | — |
| Compound 141, | 0.5 | h | 141 | 1750 ± 165 | — | — |
| 1 mg/kg, | 1 | h | | 1930 ± 416 | — | — |
| p.o., TID × | 2 | h | | 1757 ± 262 | — | — |
| 21 days | 4 | h | | 850 ± 185 | 390 ± 107 | 12.4 ± 2.79 |
| | 8 | h | | 780 ± 53.4 | — | — |
| | 10 | h | | 676 ± 42.5 | — | — |
| | 24 | h | | 33.7 ± 4.48 | 14.3 ± 4.44 | BQL |
| Group 10, | 0.25 | h | Compound | 702 ± 91.7 | — | — |
| Compound 141, | 0.5 | h | 141 | 1362 ± 270 | — | — |
| 1.5 mg/kg, | 1 | h | | 1943 ± 211 | — | — |
| p.o., BID × | 2 | h | | 2123 ± 241 | — | — |
| 21 days | 4 | h | | 950 ± 365 | 317 ± 61.6 | 10.1 ± 1.79 |
| | 8 | h | | 523 ± 19.7 | — | — |
| | 10 | h | | 328 ± 12.4 | — | — |
| | 24 | h | | 11.2 ± 2.61 | 6.70 | BQL |
| Group 11, | 0.25 | h | Compound | 1583 ± 247 | — | — |
| Compound 141, | 0.5 | h | 141 | 2850 ± 361 | — | — |
| 3 mg/kg, | 1 | h | | 5133 ± 799 | — | — |
| p.o., QD × | 2 | h | | 5290 ± 555 | — | — |
| 21 days | 4 | h | | 2433 ± 222 | 909 ± 165 | 22.9 ± 2.70 |
| | 8 | h | | 869 ± 101 | — | — |
| | 10 | h | | 617 ± 41.7 | — | — |
| | 24 | h | | 12.2 ± 1.40 | 3.01 | BQL |
| Group 12 | 0.25 | h | Compound | 2930 ± 226 | — | — |
| Compound 141, | 0.5 | h | 141 | 6303 ± 457 | — | — |
| 5 mg/kg, | 1 | h | | 6343 ± 1164 | — | — |
| p.o., BID × | 2 | h | | 6127 ± 1281 | — | — |
| 21 days | 4 | h | | 3730 ± 1139 | 1301 ± 471 | 51.4 ± 21.7 |
| | 8 | h | | 3260 ± 243 | — | — |
| | 10 | h | | 2367 ± 37.1 | — | — |
| | 24 | h | | 38.9 ± 5.18 | 79.3 | 2.25 |

Note:
^{a}BQL: Below the lower limit of quantitation.

TABLE 84
Plasma AUC at steady state (ss)

AUC at ss (Day 21) (h* ng/mL)

| | Average | SD |
|---|---|---|
| Dabrafenib, 100 | 18101 | 4690 |
| mg/kg, p.o., QD | | |
| Compound 26, 6 | 115437 | 11735 |
| mg/kg, p.o., QD | | |
| Compound 26, 1.5 | 37360 | 7749 |
| mg/kg, p.o., BID | | |
| Compound 26, 3 | 87912 | 3799 |
| mg/kg, p.o., BID | | |
| Compound 26, 10 | 311875 | 43721 |
| mg/kg, p.o., BID | | |
| Compound 26, 2 | 88320 | 10492 |
| mg/kg, p.o., TID | | |

### Example 15: Human Dose Projections for Compound 26

The first-in-human dose projections for Compound 26 were made using prediction models. These models utilized experimentally determined physicochemical and ADME parameters relevant to Compound 26. The in vivo plasma clearance (Cl) and volume of distribution (Vdss) values obtained across different preclinical species (mouse, rat, dog and monkey) were scaled to predict human values using allometry. The final plasma protein binding (PPB) corrected human Cl was 0.88 ml/min/kg and Vdss was 0.5 μL/kg. In addition, the human efficacious AUC (area under the concentration curve) was derived from a PKPD efficacy study in a tumor bearing mouse model. The maximally efficacious minimal dose (MED) in tumor bearing mouse model was determined to be 3 mg/kg BID (bis in die or twice a day), with a corresponding observed AUC of 87,912 h*ng/ml. The corresponding human efficacious AUC was predicted to be 6800 h*ng/ml. With these inputs a pharmacokinetic model (PBPK) was developed to project first in human BID dose. The oral bioavailability data for Compound 26 from preclinical species, mouse, rat, dog and monkey was simulated in the PBPK model for that species, to build confidence in the human PBPK model. The human PBPK model based on rodent species, predicted the human dose to be 18 mg BID that can achieve human efficacious AUC of 6800 h*ng/ml with human oral bioavailability of 69.4% and with an intestinal solubility set to 7.6 mg/L. The human PBPK model based on the dog, predicted the human dose to be 55 mg BID that can achieve a human efficacious AUC of 6800 h*ng/ml with human oral bioavailability of 22.6% and with an intestinal solubility set to 1.6 mg/L. Other predicted human parameters were a plasma half-life (T half) of 6.7 h. a Plasma Cmax (maximum plasma concentration) and a Ctrough (minimal plasma concentration) value were predicted to be 392 ng/ml and 167 ng/ml respectively in the human PBPK model based on rodents. Plasma Cmax and Ctrough values were predicted to be 378 ng/ml and 175 ng/ml respectively in the human PBPK model based on dog data.

TABLE 86
Oral Dose Projection for Fasted Human

| PBPK models | Oral PK | | |
|---|---|---|---|
| Compounds | Compound 26 | | |
| Dosing up to | BID (each 12 hours) | | |
| steady-state | | | |
| Human Vdss | 0.50 L/kg | | |
| Human CL | 0.88 mL/min/kg | | |
| PO datasets | Human PBPK (with | Human PBPK (with | Human PBPK (with |
| | intestinal sol. | intestinal sol. | intestinal sol. |
| | of 7.6 mg/L | of 1.6 mg/L | of 1.0 mg/L |
| | from rodents) | from dog) | from cyno) |
| Oral dose BID | 18 | 55 | 90 |
| (mg) | | | |
| Plasma T_{1/2} | 6.7 | 6.7 | 6.7 |
| (hour) | | | |
| Plasma | 392 | 378 | 379 |
| Cmax(ng/mL) | | | |
| Plasma AUC_{0-24 hours} | 6800 | 6800 | 6800 |
| (ng hour/mL) | | | |
| Plasma | 167 | 175 | 179 |
| Ctrough | | | |
| (ng/mL) | | | |
| Bioavailability | 69.4 | 22.6 | 14.0 |
| F (%) | | | |

### Example 16: A375 Cell Viability Assay (CTGlo)

The effect of compounds of the invention on cell viability was assessed based on the quantification of ATP using the CellTiter-Glo® 2.0 (referred to here as CTGlo) Cell Viability Assay (Catalog No. G9243, Promega, Madison, WI, USA), which signals the presence of metabolically active cells. The A375 human melanoma cell line used in these assays exhibits epithelial morphology, it was isolated from the skin of a 54-year-old, female patient with malignant melanoma.

A375 cells were obtained from ATCC (Catalog No. CRL-1619, Manassas, VA, USA) (also referred to here as A375.1) and were maintained in DMEM, (Corning, Catalog No. 10-017-CV, Glendale, AZ, USA) supplemented with 10% fetal bovine serum (FBS; Catalog No. 16000044, Gibco, Grand Island, NY, USA) at 37° C. in an atmosphere of 5% CO₂ in air. The cells were routinely sub-cultured to maintain cell density between 3×10⁵-1.5×10⁶ cells/mL. Cells were washed with phosphate buffered saline, pH 7.4 (PBS; Catalog No. 10010049, ThermoFisher Scientific, Waltham, MA, USA), trypsinized with Trypsin-EDTA (0.25%), phenol red (Catalog No. 25200056, ThermoFisher Scientific, Waltham, MA, USA) for 5 min at 37° C., and resuspended in growth media. An aliquot of cell suspension was diluted 2× with a 0.4% Trypan Blue solution (Catalog No. 15250061, ThermoFisher Scientific, Waltham, MA, USA) and cell count was determined. Cell concentration was adjusted with growth media to 2.0×10⁴ cells/mL.

To determine the effect of compounds on cell viability, 50 μL A375 cells suspended in growth media at 2.0×10⁴ cells/mL (for a cell density of 1000 cells/well) were dispensed to each well of a 384-well, black TC-treated microplates (Catalog No. 3571, Corning, Glendale, CA, USA).

Test compounds were prepared by dissolving neat compounds in dimethyl sulfoxide (DMSO; Catalog No. D8418, Sigma-Aldrich, Inc., St. Louis, MO, USA) to generate 10 mM stock solution and stored at −20° C. The 10 mM DMSO stock solutions were serially diluted (half log titration) in DMSO to generate a 10-point dose series with a final concentration of 10 μM, in duplicate. Using an Echo 550 Acoustic Liquid Handler (Beckman Coulter Life Sciences, Indianapolis, IN, USA), plated cells were treated with 50 nL of serially diluted test compound solutions in duplicate. 50 nL DMSO was transferred to control wells. Plates were incubated at 37° C., 5% C₀₂ for 72 hours. CellTiter-Glo® reagent was then added to the cells and luminescence signals were measured after 4 hours using an EnVision™ Multimode plate reader (Perkin Elmer, Santa Clara, CA).

For data normalization, cells untreated with test compounds at Time=96 hours were set to 100% (equivalent to maximal cell growth after 96 hours). Media-only wells were set to −100% (equivalent to cytotoxicity). Percent Viability was determined by normalizing the signal with positive and DMSO treated negative controls on the same microtiter plate. The half-maximal inhibition of cell growth (GI₅₀) was computed from where the fitted curve crosses 50%. The Emax, or maximum effect of each compound, represents the lowest % viability achieved following compound treatment

The experiment was conducted at least twice for Compound 26, Compound 141, Compound 26-NMe, and Compound 141-NMe. The results are shown in FIG. 83. The data shown in the figure is the average of two or more experiments.

The compounds designated as —NMe are identical to the parent compound with the exception of a methyl group added to the nitrogen group in the cereblon binding portion of the bifunctional degrader. This chemical modification prevents the formation of a functional ternary complex between BRAF V600E, the compound, and cereblon, therefore blocking ubiquitination and degradation of the BRAF V600E protein. The effect of the —NMe compound on viability therefore demonstrates the inhibitory contribution of the compound. The parental compounds Compound 26 and Compound 141 have a significant impact on the viability of A375 cells, whereas the —NMe modified compounds have minimal impact on viability at most doses. This suggests that most of the effect of Compound 26 and Compound 141 on cellular viability is due to the degradation of BRAF V600E in A375 cells, and not inhibition.

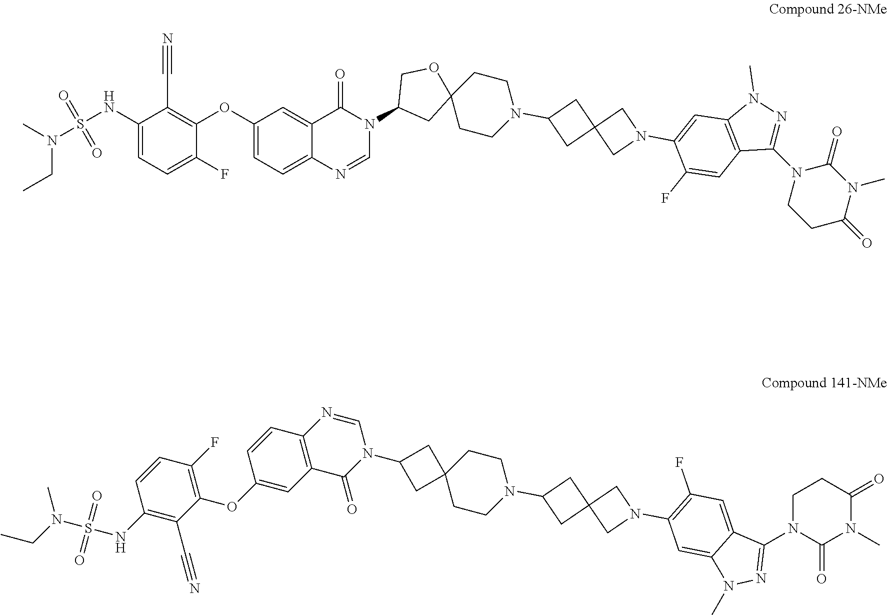

### Example 17: LM-MEL-42 Cell Viability Assay

The effect of selected compounds of the invention on cell viability in a V600K mutant BRAF cell line was assessed based on the quantification of ATP using the CellTiter-Glo® 2.0 (referred to here as CTGlo) Cell Viability Assay (Catalog No. G9243, Promega, Madison, WI, USA), which signals the presence of metabolically active cells. The LM-MEL-42 human melanoma cell line used in these assays exhibits epithelial morphology and was isolated from the spleen of a 67-year-old, male patient with malignant melanoma.

LM-MEL-42 cells were obtained from ATCC (Catalog No. CRL-3335, Manassas, VA, USA) (also referred to here as LMMEL42.1) and were maintained RPMI-1640 (ATCC® 30-2001™) supplemented with 10% fetal bovine serum (FBS; Catalog No. 16000044, Gibco, Grand Island, NY, USA) at 37° C. in an atmosphere of 5% C₀₂ in air. The cells were routinely sub-cultured to maintain cell density between 3×10⁵−1.5×10⁶ cells/mL. Cells were washed with phosphate buffered saline, pH 7.4 (PBS; Catalog No. 10010049, ThermoFisher Scientific, Waltham, MA, USA), trypsinized with Trypsin-EDTA (0.25%), phenol red (Catalog No. 25200056, ThermoFisher Scientific, Waltham, MA, USA) for 5 min at 37° C., and resuspended in growth media. An aliquot of cell suspension was diluted 2× with a 0.4% Trypan Blue solution (Catalog No. 15250061, ThermoFisher Scientific, Waltham, MA, USA) and cell count was determined. Cell concentration was adjusted with growth media to 2.0×10⁴ cells/mL.

To determine the effect of compounds on cell viability, 50 μL LM-MEL-42 cells suspended in growth media at 4.0×10⁴ cells/mL (for a cell density of 2000 cells/well) were dispensed to each well of a 384-well, black TC-treated microplates (Catalog No. 3571, Corning, Glendale, CA, USA).

Test compounds were prepared by dissolving neat compounds in dimethyl sulfoxide (DMSO; Catalog No. D8418, Sigma-Aldrich, Inc., St. Louis, MO, USA) to generate 10 mM stock solution and stored at −20° C. The 10 mM DMSO stock solutions were serially diluted (half log titration) in DMSO to generate a 10-point dose series with a final concentration of 10 μM, in duplicate. Using an Echo 550 Acoustic Liquid Handler (Beckman Coulter Life Sciences, Indianapolis, IN, USA), plated cells were treated with 50 nL of serially diluted test compound solutions in duplicate. 50 nL DMSO was transferred to control wells. Plates were incubated at 37° C., 5% CO₂ for 72 hours. CellTiter-Glo® reagent was then added to the cells and luminescence signals were measured after 4 hours using an EnVision™ Multimode plate reader (Perkin Elmer, Santa Clara, CA).

For data normalization, cells untreated with test compounds at Time=96 hours were set to 100% (equivalent to maximal cell growth after 96 hours). Media-only wells were set to −100% (equivalent to cytotoxicity). Percent Viability was determined by normalizing the signal with positive and DMSO treated negative controls on the same microtiter plate. The half-maximal inhibition of cell growth (GI50) was computed from where the fitted curve crosses 50%. The Emax, or maximum effect of each compound, represents the lowest % viability achieved following compound treatment The study was conducted in triplicate

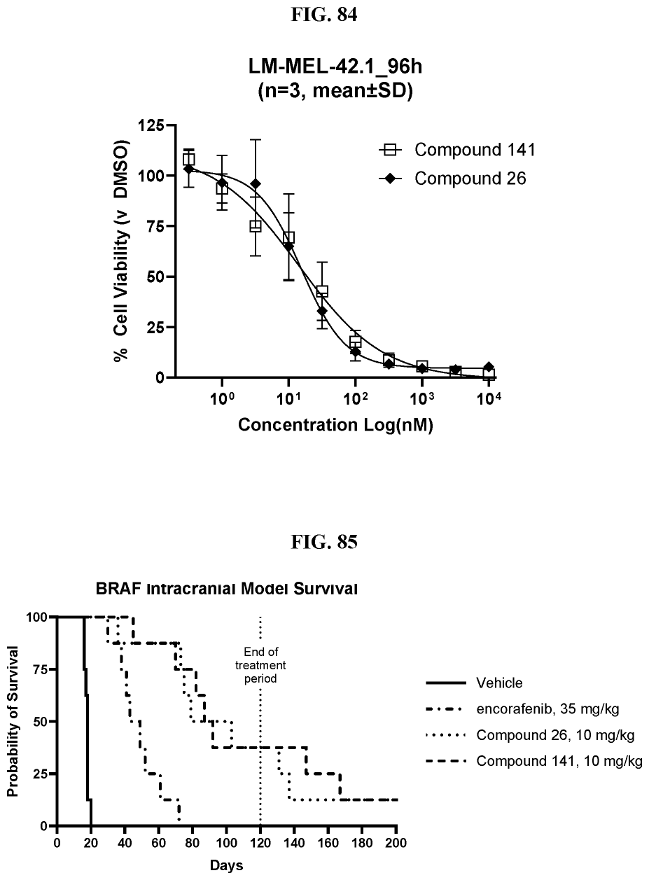

The experimental results are shown in FIG. 84. The results support that the compounds inhibit cellular growth of melanoma cells dependent on the BRAF-V600K mutation.

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modification may be made thereto without departing from the spirit or scope of the invention as defined in the claims.

We claim:

1. A compound of Formula: 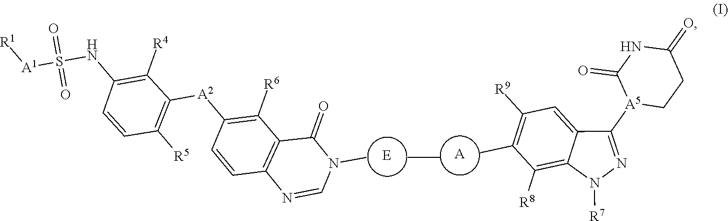
or a pharmaceutically acceptable salt thereof;
wherein:
Cycle \textcircled{D} is a spiroheterocyclyl containing 1 or 2 nitrogen atoms and containing no other heteroatoms or piperidine-1,4-diyl, wherein cycle \textcircled{D} is optionally substituted with one or two substituents independently selected from the group consisting of halogen, alkyl, and alkoxy;
Cycle \textcircled{E} is a spiroheterocyclyl containing 1 nitrogen atom and 1 oxygen atom and containing no other heteroatoms, wherein cycle \textcircled{E} is optionally substituted with one or two substituents independently selected from the group consisting of halogen, alkyl, and alkoxy;
A¹ is —NR²— or —CHR^{2′}—;
R¹ is hydrogen or alkyl;
R² is selected from the group consisting of hydrogen, alkyl, and haloalkyl;
or R¹ and R² together with the nitrogen atom to which they are attached, form heterocycloalkyl optionally substituted with one or two R³ substituent(s);
R^{2′} is selected from the group consisting of hydrogen, alkyl, and haloalkyl;
or R¹ and R^{2′} together with the carbon atom to which they are attached, form cycloalkyl optionally substituted with one or two R³ substituent(s);
each R³ is independently selected from the group consisting of hydrogen, halogen, alkyl, and alkoxy;
R⁴ is selected from the group consisting of hydrogen, alkyl, cyano, and halogen;
R⁵ is selected from the group consisting of hydrogen, alkyl, cyano, and halogen;
A² is selected from the group consisting of —O—, —NH—, and —(C═O)—;
R⁶ is selected from the group consisting of hydrogen, halogen, hydroxy, amino, dialkylamino, alkoxy, alkyl, and alkoxyalkyl;
R⁷ is hydrogen or alkyl;
R⁸ is selected from the group consisting of hydrogen, alkyl, cyano, halogen, and alkoxy;
R⁹ is selected from the group consisting of hydrogen, alkyl, cyano, halogen, and alkoxy; and
A⁵ is CH or N.

2. The compound of claim 1, wherein Cycle \textcircled{E} is

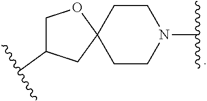 [SMILES: *C1COC2(CCN(*)CC2)C1]

3. The compound of claim 1, wherein Cycle \textcircled{E} is

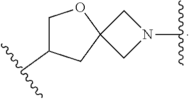 [SMILES: *C1COC2(C1)CN(*)C2]

4. The compound of claim 1, wherein Cycle \textcircled{D} is

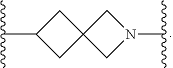 [SMILES: *C1CC2(C1)CN(*)C2]

5. The compound of claim 1, wherein A⁵ is N.

6. The compound of claim 1, wherein R⁷ is CH₃.

7. The compound of claim 1, wherein R⁷ is alkyl.

8. The compound of claim 1, wherein R⁸ is hydrogen.

9. The compound of claim 1, wherein R⁹ is halogen or cyano.

10. The compound of claim 1, wherein R⁹ is F.

11. The compound of claim 1, wherein A¹ is NCH₃.

12. The compound of claim 1, wherein R¹ is ethyl.

13. The compound of claim 2, wherein Cycle \textcircled{D} is

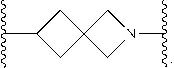 [SMILES: *C1CC2(C1)CN(*)C2]

14. The compound of claim 13, wherein A² is O.

15. The compound of claim 14, wherein R⁴ is cyano.

16. The compound of claim 15, wherein R⁵ is F.

17. The compound of claim 16, wherein R⁶ is hydrogen.

18. The compound of claim 17, wherein A⁵ is N.

19. The compound of claim 17, wherein R⁷ is CH₃.

20. The compound of claim 17, wherein R⁸ is hydrogen.

21. The compound of claim 17, wherein R⁹ is F.

22. The compound of claim 17, wherein R¹ is ethyl.

23. The compound of claim 17, wherein A¹ is NCH₃.

24. The compound of claim 23, wherein A⁵ is N.

25. The compound of claim 24, wherein R¹ is ethyl

26. The compound of claim 1, wherein the compound is 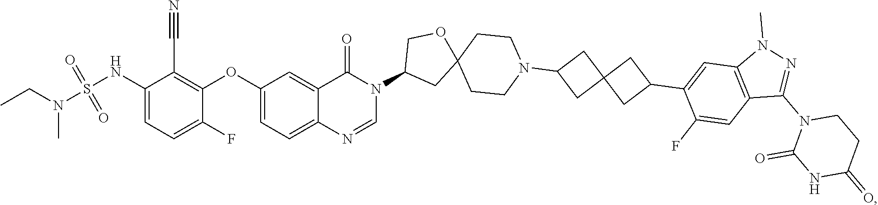 [SMILES: CCN(C)S(=O)(=O)Nc1ccc(F)c(Oc2ccc3ncn([C@H]4COC5(CCN(C6CC7(CC(c8cc9c(cc8F)c(N8CCC(=O)NC8=O)nn9C)C7)C6)CC5)C4)c(=O)c3c2)c1C#N]
or a pharmaceutically acceptable salt thereof.

27. The compound of claim 1, wherein the compound is 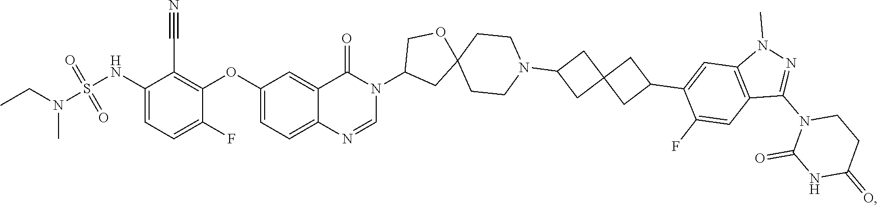 [SMILES: CCN(C)S(=O)(=O)Nc1ccc(F)c(Oc2ccc3ncn(C4COC5(CCN(C6CC7(CC(c8cc9c(cc8F)c(N8CCC(=O)NC8=O)nn9C)C7)C6)CC5)C4)c(=O)c3c2)c1C#N]
or a pharmaceutically acceptable salt thereof.

28. The compound of claim 1, wherein the compound is 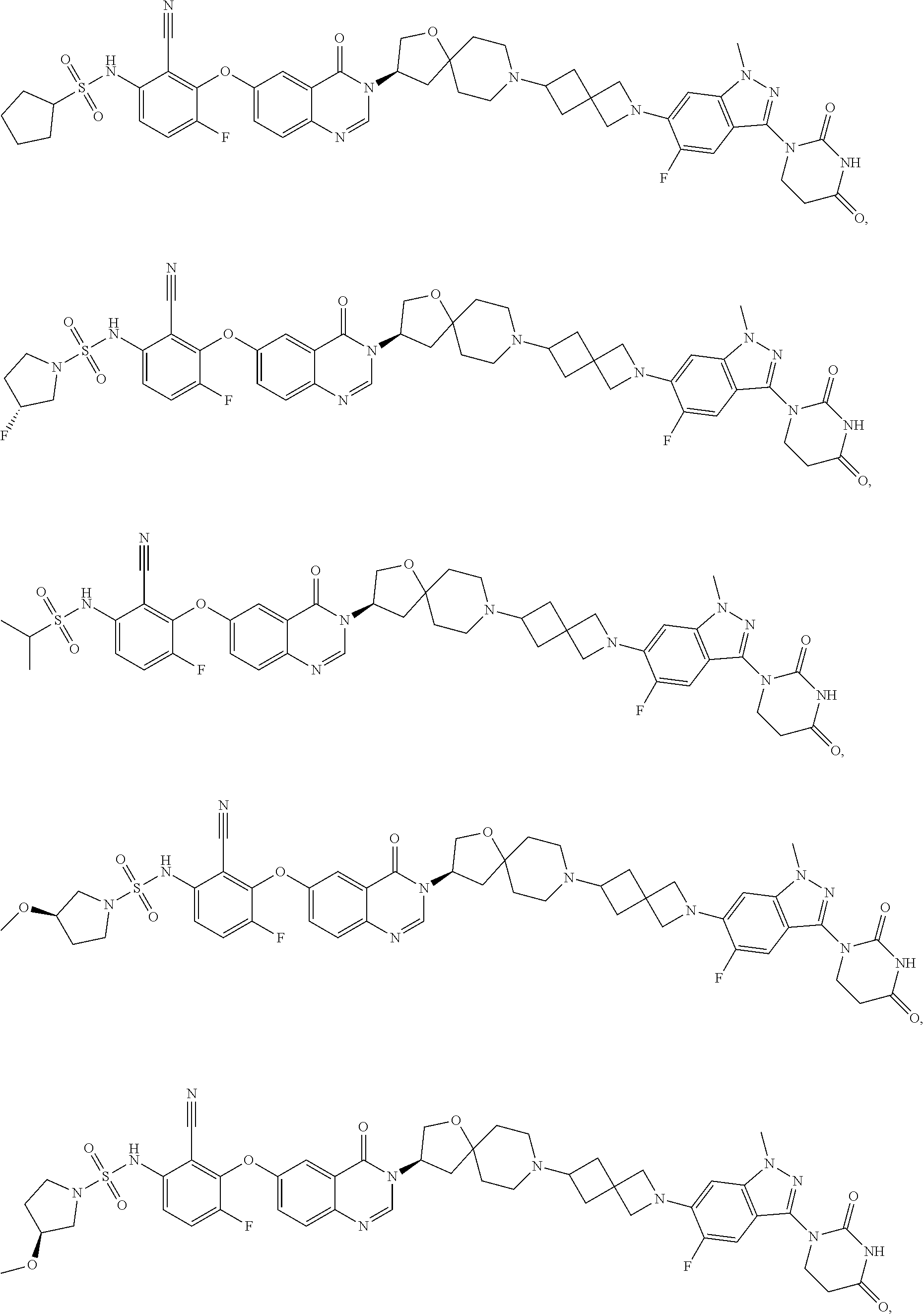 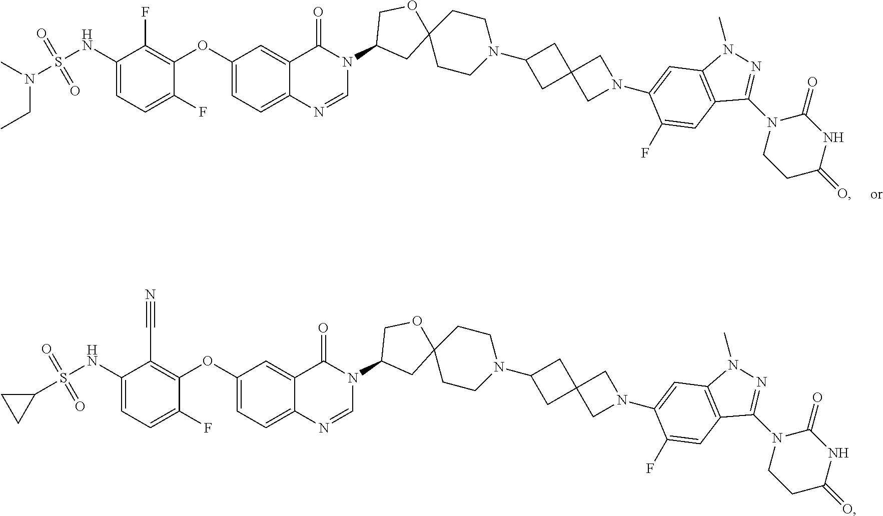 [SMILES: CCN(C)S(=O)(=O)Nc1ccc(F)c(Oc2ccc3ncn([C@H]4COC5(CCN(C6CC7(C6)CN(c6cc8c(cc6F)c(N6CCC(=O)NC6=O)nn8C)C7)CC5)C4)c(=O)c3c2)c1F ; Cn1nc(N2CCC(=O)NC2=O)c2cc(F)c(N3CC4(CC(N5CCC6(CC5)C[C@@H](n5cnc7ccc(Oc8c(F)ccc(NS(=O)(=O)C9CC9)c8C#N)cc7c5=O)CO6)C4)C3)cc21]
or a pharmaceutically acceptable salt thereof.

29. The compound of claim 1, wherein the compound is 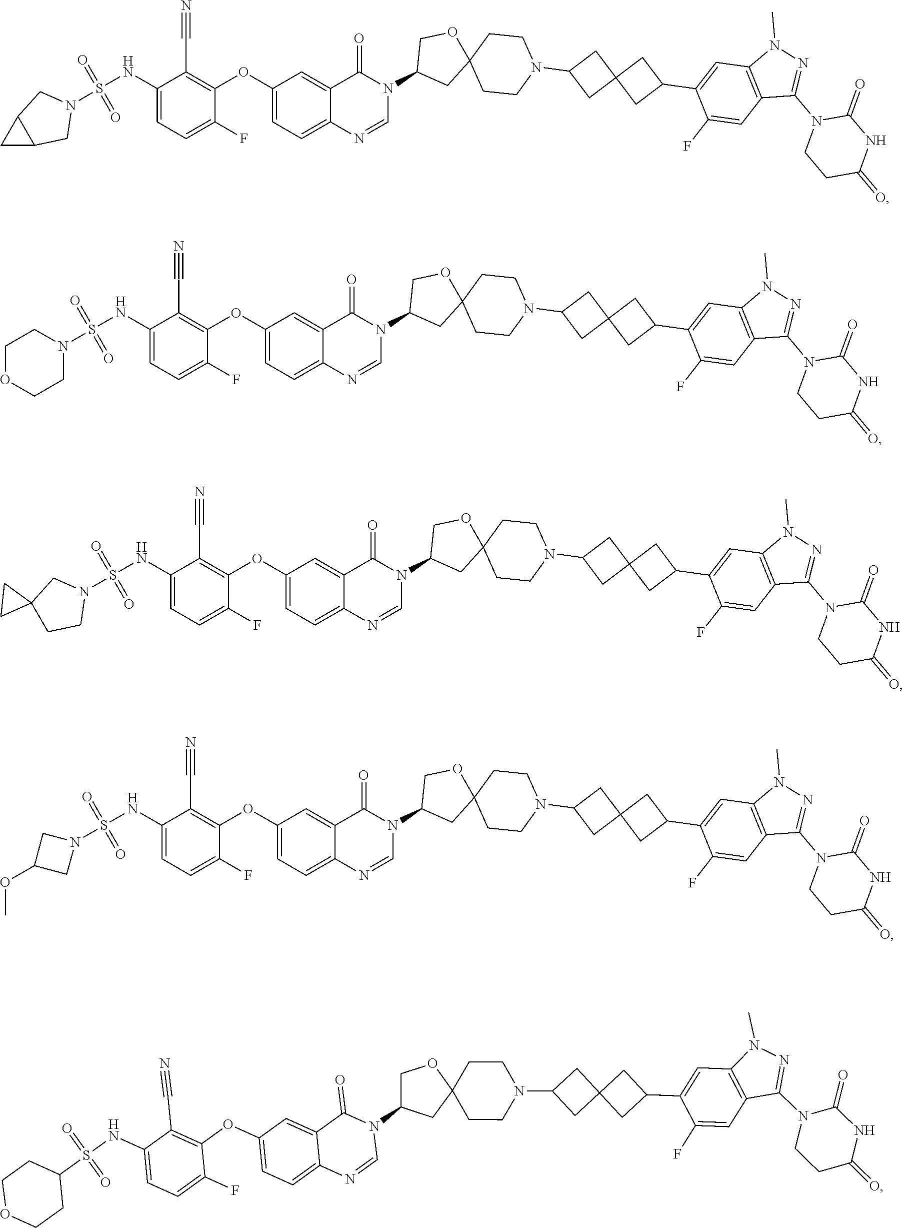
or a pharmaceutically acceptable salt thereof.

30. The compound of claim 1, wherein the compound is 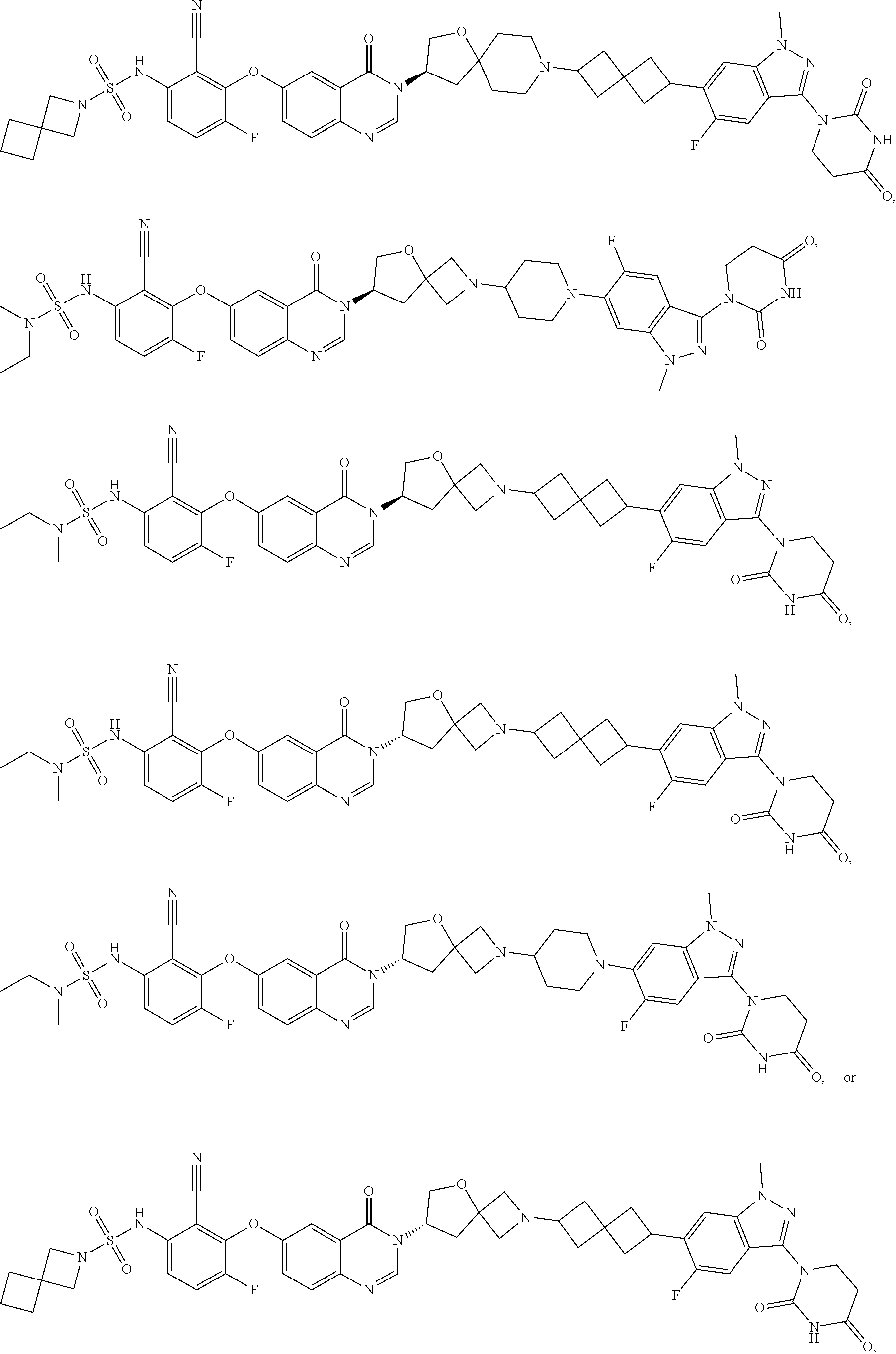
or a pharmaceutically acceptable salt thereof.
